(19) United States
(12) Patent Application Publication
(10) Pub. No.: US 2026/0191968 A1
(43) Pub. Date: Jul. 9, 2026

(54) PROTAC DEGRADERS OF MLLT1 AND/OR MLLT3

(71) Applicant: DARK BLUE THERAPEUTICS LTD, Oxford (GB)

(72) Inventors: Iain SIMPSON, Oxford (GB); Sarah MAJOR, Oxford (GB); Benjamin RAHEMTULLA, Oxford (GB); Ryan TINSON, Oxford (GB); Katherine JONES, Oxford (GB); Charlene FALLAN, Oxford (GB)

(73) Assignee: DARK BLUE THERAPEUTICS LTD, Thousand Oaks (GB)

(21) Appl. No.: 19/557,931

(22) Filed: Mar. 5, 2026

Related U.S. Application Data
(63) Continuation of application No. PCT/EP2025/067413, filed on Jun. 20, 2025.

(30) Foreign Application Priority Data
Jun. 21, 2024 (EP) 24183691.5
Jun. 21, 2024 (EP) 24183693.1

(51) Int. Cl. A61K 47/54; A61P 35/02

(52) U.S. Cl. CPC A61K 47/545; A61P 35/02

(57) ABSTRACT

The invention relates to a compound which is a Proteolysis Targeting Chimera (PROTAC) or a pharmaceutically acceptable salt thereof, wherein the PROTAC has the structure:

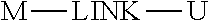

wherein U is an E3 ubiquitin ligase binding moiety, LINK is a moiety that covalently links M and U, and M is an MLLT1 and/or MLLT3 binder of formula (I):

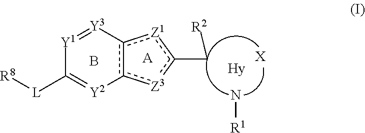

wherein:
wherein Z¹, Z², Y¹, Y², Y³, R¹, R², R⁸, X, L and Hy are as defined herein, and either R⁸ is a bond to LINK, or M is bonded to LINK via a C or N atom within group R⁸ or ring Hy such that a hydrogen atom on the C or N atom within group R⁸ or ring Hy is replaced with a bond to LINK. The compounds are useful in the treatment of cancer.

### CROSS REFERENCE TO RELATED APPLICATIONS

This application is a continuation of International Application No. PCT/EP2025/067413, filed Jun. 20, 2025, which claims the benefit of priority to EP Patent Application No. 24183691.5, filed Jun. 21, 2024 and to EP Patent Application No. 24183693.1, filed Jun. 21, 2024.

### FIELD OF THE INVENTION

The present invention relates to compounds that find use in the treatment of cancer by inducing selective degradation of MLLT1 and/or MLLT3. The invention also provides such compounds per se, pharmaceutical compositions comprising such compounds, and methods of treating cancer by administering such compounds.

### BACKGROUND

The processes controlling gene transcription are highly regulated during development and normal homeostasis. Dysregulation of gene transcription is a common driver of cancer with somatic defects in proteins that control gene transcription a frequent occurrence (Cell, 2013, 153, 17; Cell, 2017, 168, 629). Transcriptional elongation is a central step in gene transcription, carried out by the RNA polymerase II (PolII), which itself is kept under tight control by regulatory protein complexes. During development PolII is recruited to sites of the genome proximal to the transcription start site for target genes and is kept in a paused state. Such target genes are often immediate response genes such as heat-shock genes and key developmental genes. Productive elongation is initiated by the coordinated interplay of regulatory protein complexes including the super elongation complex (SEC). The SEC includes multiple proteins with diverse functions including protein phosphorylation, histone reader, histone modification and regulatory activities. Critical to the recruitment of the SEC to target genes are the histone reader proteins MLLT1 (mixed lineage leukaemia translocated to 1, also known as eleven-nineteen leukaemia, ENL) and MLLT3 (mixed lineage leukaemia translocated to 3, also known as AF-9). MLLT1 and 3 contain essential YEATS domains that bind acetylated histones. Mutations in the YEATS domains reduce loading of Poll on to SEC target genes and suppression of gene transcription. (Cell Mol Life Sci 2018, 75, 3931; Nat Rev Mol Cell Biol 2012, 13, 543).

Gain-of-function mutations in the MLLT1 Yeats domain have been causally associated with Wilm's tumor (also known as nephroblastoma) a kidney cancer most commonly observed in children (Nature 2020, 577, 121). Additionally, it has been shown that for certain acute leukaemias MLLT1 represents a critical dependency (Nature 2017, 543, 270). Such leukaemias include mixed-lineage leukaemia (MLL) rearranged leukaemia. MLL rearrangements arise from in frame fusions of the MLL gene with more than 80 different partner genes, many of which are involved in the regulation of transcription elongation including components of the SEC (Front. Pediatr. 5:4. doi: 10.3389/fped.2017.00004). MLL rearrangements are observed in approximately 10% of all acute leukaemias including a high frequency in infantile ALL where it accounts for 70-80% of all cases (Front. Pediatr. 5:4. doi: 10.3389/fped.2017.00004). Notably it is reported that patients with MLL rearranged leukaemias have an especially poor prognosis (New Engl J Med 2016, 374, 2209). Target genes for MLL-SEC complexes include potent oncogenes such as BCL-2, Myc and CDK6 along with many other genes implicated in maintaining cancer cell self-renewal, growth and survival, such as the HOX family genes and MEIS1 (Front. Pediatr. 5:4. doi: 10.3389/fped.2017.00004). Consistent with a role in the regulation of multiple oncogenes, it has been demonstrated that the SEC can play a critical role in the transcriptional addiction of both hematopoietic and solid cancers. For example, data supports potential in non-MLL rearranged leukaemia (Cancer Disc 2022, 12, 2684) and it is reported that with some breast cancer cells, growth and survival is dependent on a SEC mediated transcription of the Myc oncogene (Cell Rep. 2021 Feb. 16; 34(7):108749. doi: 10.1016/j.celrep.2021.108749. PMID: 33596420; PMCID: PMC8006859).

Taken together the evidence supports MLLT1 and/or MLLT3 as an attractive therapeutic target across acute leukaemias and solid cancers.

Proteolysis targeting chimeras (PROTACs) have been proposed as a small molecule-based platform technology capable of inducing proteolysis of a target protein in the body. The PROTAC is a bifunctional compound in which a molecule that binds to a disease-related target protein and an E3 ubiquitin ligase binding moiety are linked by a chemical linker. Theoretically, the PROTAC compound is capable of inducing degradation of the target protein by placing the disease-related target protein near the E3 ubiquitin ligase.

Drug discovery efforts have resulted in agents that either bind the YEATS domain to block the MLLT1 and/or MLLT3 protein to histone interaction or that result in degradation of the MLLT1 and/or MLLT3 protein (using a PROTAC approach). In all cases however, the reported activity has been weak (ACS Cent Sci 2021, 7, 815; Cancer Disc 2022, 12, 2684; Angew. Chem. Int. Ed. 2018, 57, 16302). Accordingly, there remains a high need to identify potent modulators of MLLT cell function.

### SUMMARY OF THE INVENTION

The inventors have discovered a series of compounds that induce selective degradation of MLLT1 and/or MLLT3. Accordingly, the invention provides a compound which is a Proteolysis Targeting Chimera (PROTAC) or a pharmaceutically acceptable salt thereof, wherein the PROTAC has the structure:

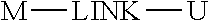

wherein U is an E3 ubiquitin ligase binding moiety, LINK is a moiety that covalently links M and U, and M is an MLLT1 and/or MLLT3 binder of formula (I):

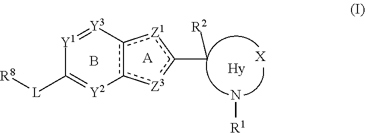

wherein:
one of Z¹ and Z³ is —N(H)— and the other is N or —C(R⁴)—, Y¹ is N, Y² is N or —C(R⁶)—, and Y³ is N or —C(R⁵)—;
Hy is a 4- to 7-membered heterocyclic ring containing X and at least one N atom, wherein: ring Hy is linked to ring A via a C atom within ring Hy, said C atom also being linked to R²; a N atom within ring Hy is substituted by R¹; X is a bond, —N(R¹¹)—, O, S, —S(O)₂—, —S(O)(NR¹¹)—, or —C(R¹¹)₂—; and the rest of ring Hy is unsubstituted or substituted by one or two R³;
L is —C(O)N(H)—, wherein the C atom of L is bonded to R⁸, and the N atom of L is bonded to ring B;
R¹ is H, C₁₋₄ cycloalkyl, or C₁₋₄ alkyl which is itself unsubstituted or substituted with one C₁₋₄ alkoxy or one, two or three halo;
R² is H or methyl;
each R³ is independently selected from C₁₋₄ alkyl, C₁₋₄ alkoxy, phenyl, a 5- to 6-membered heteroaryl ring and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring or a C₃₋₆ heterocycloalkyl ring;
R⁴ and R⁶ are independently selected from H, halo, CN, C₁₋₄ alkoxy, and C₁₋₄ alkyl which is itself unsubstituted or substituted by one, two or three halo;
R⁵ is selected from H, halo, C₁₋₄ alkoxy, C₃₋₅ cycloalkyl and C₁₋₄ alkyl which is itself unsubstituted or substituted by one, two or three halo;
R⁸ is (i) a bond to LINK or (ii) a group selected from a 6-membered aryl ring, a 5- to 6-membered heteroaryl ring, a 5- to 6-membered heterocyclyl ring, and a 5- to 6-membered cycloalkyl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo; and
each R¹¹ is independently selected from H, C₁₋₄ alkyl, and C₁₋₄ cycloalkyl;
wherein either R⁸ is (i) a bond to LINK, or R⁸ is a group (ii) and M is bonded to LINK via a C or N atom within group R⁸ such that a hydrogen atom on the C or N atom within group R⁸ is replaced with a bond to LINK.

In a preferred embodiment, M is of formula (II):

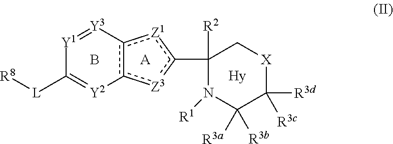

wherein:
Z¹, Z³, Y¹, Y², Y³, R¹, R², R⁸, X and L are as defined herein;
R^{3a} and R^{3b} are independently selected from H, C₁₋₄ alkyl, phenyl, a 5- to 6-membered heteroaryl ring and C₁₋₄ alkoxy, or R^{3a} and R^{3b} form, together with the C atom to which they are attached, a C₃₋₆ cycloalkyl ring or a C₃₋₆ heterocycloalkyl ring,
R^{3c} and R^{3d} are independently selected from H, C₁₋₄ alkyl, halo, phenyl, a 5- to 6-membered heteroaryl ring and C₁₋₄ alkoxy, or R^{3c} and R^{3d} form, together with the C atom to which they are attached, a C₃₋₆ cycloalkyl ring or a C₃₋₆ heterocycloalkyl ring, or
R^{3a} and R^{3c} are H, and R^{3b} and R^{3d} form, together with the C atoms to which they are attached, a C₅₋₆ cycloalkyl ring, with the proviso that when X is —N(R¹¹)—, O, S, —S(O)₂— or —S(O)(NR¹¹)-then neither R^{3c} nor R^{3d} are halo;
wherein at least two of R^{3a}, R^{3b}, R^{3c} and R^{3d} are H; and
wherein either R⁸ is (i) a bond to LINK, or R⁸ is a group (ii) and M is bonded to LINK via a C or N atom within group R⁸ such that a hydrogen atom on the C or N atom within group R⁸ is replaced with a bond to LINK.

In a more preferred embodiment, M is of formula (III):

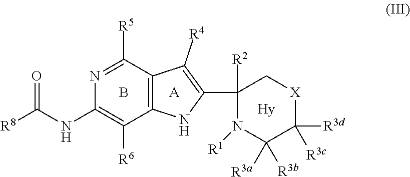

wherein:
R¹ is H or C₁₋₄ alkyl which is itself unsubstituted or substituted with one C₁₋₄ alkoxy or one, two or three halo, preferably with one C₁₋₄ alkoxy;
R² is H or methyl;
R^{3a} and R^{3b} are independently selected from H, C₁₋₄ alkyl, and C₁₋₄ alkoxy, or R^{3a} and R^{3b} form, together with the C atom to which they are attached, a C₃₋₆ cycloalkyl ring,
R^{3c} and R^{3d} are independently selected from H, C₁₋₄ alkyl, halo and C₁₋₄ alkoxy, or R^{3c} and R^{3d} form, together with the C atom to which they are attached, a C₃₋₆ cycloalkyl ring, or
R^{3a} and R^{3c} are H, and R^{3b} and R^{3d} form, together with the C atoms to which they are attached, a C₅₋₆ cycloalkyl ring,
with the proviso that when X is —N(Me)- or O, then neither R^{3c} nor R^{3d} are halo;
wherein at least two of R^{3a}, R^{3b}, R^{3c} and R^{3d} are H;
X is a bond, —N(Me)-, O or —CH₂—;
R⁴ is selected from H, CN, halo, C₁₋₄ alkoxy, and C₁₋₄ alkyl which is itself unsubstituted or substituted by one, two or three halo;
R⁵ and R⁶ are independently selected from H, halo, C₁₋₄ alkoxy, and C₁₋₄ alkyl which is itself unsubstituted or substituted by one, two or three halo;
R⁸ is (i) a bond to LINK or (ii) a group selected from a 6-membered aryl ring, a 5- to 6-membered heteroaryl ring, and a 5- to 6-membered heterocyclyl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo; and
wherein either R⁸ is (i) a bond to LINK, or R⁸ is a group (ii) and M is bonded to LINK via a C or N atom within group R⁸ such that a hydrogen atom on the C or N atom within group R⁸ is replaced with a bond to LINK.

In a particularly preferred embodiment, M is of formula (IV):

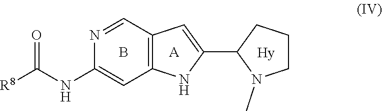

wherein R⁸ is (i) a bond to LINK or (ii) a group selected from a 6-membered aryl ring and a 5-to 6-membered heteroaryl ring, the group R⁸ being unsubstituted or substituted by one, two or three substituents independently selected from halo, C₁₋₄ alkyl and R¹⁰;
R¹⁰ is a 3-membered cycloalkyl ring;
wherein either R⁸ is (i) a bond to LINK, or R⁸ is a group (ii) and M is bonded to LINK via a C or N atom within group R⁸ such that a hydrogen atom on the C or N atom within group R⁸ is replaced with a bond to LINK. As discussed elsewhere herein, the stereochemistry of the compounds of the invention at the ring Hy is preferably such that the bond from ring Hy to the core ring A is in the “up” position and the bond from ring Hy to R² is the down position. Thus, formula (IV) preferably has the stereochemistry depicted below:

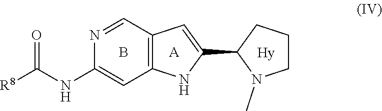

The present invention also provides a compound as described herein for use in a method of treating cancer in a subject in need thereof. Also provided is a method for treating cancer in a subject, which method comprises administering to said subject an effective amount of a compound as described herein. Further provided is the use of a compound as described herein in the manufacture of a medicament for use in treating cancer in a subject.

### BRIEF DESCRIPTION OF THE DRAWINGS

FIG. 1: Graph showing reduction in tumour growth in in vivo mouse model of human AML, following oral administration of the compound of Example 55, following the procedure described in Example 236.

### DETAILED DESCRIPTION OF THE INVENTION

### Definitions

As used herein, a C₁₋₄ alkyl group is a linear or branched alkyl group containing from 1 to 4 carbon atoms. A C₁₋₄ alkyl group is often a C₁₋₃ alkyl group. Examples of C₁₋₄ alkyl groups include methyl, ethyl, n-propyl, iso-propyl, n-butyl, sec-butyl, and tert-butyl. A C₁₋₃ alkyl group is typically a C₁₋₂ alkyl group. A C₁₋₂ alkyl group is methyl or ethyl, typically methyl. For the avoidance of doubt, where multiple alkyl groups are present, the alkyl groups may be the same or different.

As used herein, a C₁₋₄ alkoxy group is typically a said C₁₋₄ alkyl group which is joined to the rest of the molecule via an oxygen atom. Typically, a C₁₋₄ alkoxy group is a C₁₋₃ alkoxy group. Examples of C₁₋₄ alkoxy groups include methoxy, ethoxy, propoxy and butoxy. Typically, a C₁₋₃ alkoxy group is a C₁₋₂ alkoxy group such as a methoxy or ethoxy group. For the avoidance of doubt, where two alkoxy groups are present, the alkoxy groups may be the same or different.

As used herein, a C₁₋₃₀ alkylene group is a linear or branched divalent alkyl group that contains from 1 to 30 carbon atoms. A C₁₋₃₀ alkylene group is sometimes a C₁₋₂₀ alkylene group, and often a C₁₋₁₀ alkylene group. C₁₋₃₀ alkylene groups, C₁₋₂₀ alkylene groups and C₁₋₁₀ alkylene groups are preferably linear.

A C₁₋₃₀ alkylene group is sometimes a C₁₋₆ alkylene group, typically a C₁₋₄ alkylene group or a C₁₋₃ alkylene group. Examples of C₁₋₄ alkylene groups include methylene, ethylene, n-propylene, iso-propylene, n-butylene, sec-butylene, and tert-butylene. A C₁₋₃ alkylene group is typically a C₁₋₂ alkylene group. A C₁₋₂ alkylene group is methylene or ethylene, typically methylene. For the avoidance of doubt, where multiple alkylene groups are present, the alkylene groups may be the same or different.

As used herein, a C₂₋₆ alkenylene group is a linear or branched divalent alkenyl group containing from 2 to 6 carbon atoms and having one or more, e.g. one or two, typically one double bonds. Typically, a C₂₋₆ alkenylene group is a C₂₋₄ alkenylene group. Examples of C₂₋₄ alkenylene groups include divalent ethenylene, propenylene and butenylene. For the avoidance of doubt, where multiple alkenylene groups are present, the alkenylene groups may be the same or different.

An alkyl, alkoxy, alkylene or alkenylene group as used herein may be unsubstituted or substituted. Unless otherwise stated, substituted alkyl, alkoxy, alkylene or alkenylene groups typically carry one or more, e.g. one, two or three e.g. one, or two, e.g. one substituent selected from halo, OH, and unsubstituted C₁₋₄ alkoxy. Preferred substituents are halo and C₁₋₄ alkoxy unless otherwise stated. The substituents on a substituted alkyl, alkoxy, alkylene or alkenylene group are typically themselves unsubstituted. Where more than one substituent is present, these may be the same or different.

As used herein, a halo typically refers to chlorine, fluorine, bromine or iodine, preferably chlorine, bromine or fluorine, more preferably chorine or fluorine, most preferably fluorine unless otherwise stated.

A C₃₋₈ cycloalkyl ring is a cyclic hydrocarbon containing from 3 to 8 carbon atoms. A cycloalkyl ring may be saturated or partially unsaturated, but is typically saturated. A C₃₋₈ cycloalkyl ring is typically a C₃₋₆ cycloalkyl ring. A partially unsaturated cycloalkyl ring is a cyclic hydrocarbon containing 1 or 2, e.g. 1 double bond. C₃₋₆ cycloalkyl and C₅₋₆ cycloalkyl rings may also be referred to herein as 3- to 6-membered cycloalkyl rings and 5- to 6-membered cycloalkyl rings respectively.

A C₃₋₆ cycloalkyl ring may be a saturated C₃₋₆ cycloalkyl ring. A C₃₋₆ cycloalkyl ring may be a C₅₋₆ cycloalkyl ring, in particular a saturated C₅₋₆ cycloalkyl ring. Examples of C₃₋₆ cycloalkyl rings are cyclopropyl, cyclobutyl cyclopentyl and cyclohexyl groups.

A C₃₋₈ cycloalkyl ring may be a C₇₋₈ cycloalkyl ring, in particular a saturated C₇₋₈ cycloalkyl ring. Examples of C₇₋₈ cycloalkyl rings are cycloheptanly, cyclooctanyl, bicyclo[2.2.1]heptanyl and bicyclo[2.2.2]octanyl groups.

A 4- to 7-membered heterocyclyl ring is a cyclic group containing from 4 to 7 atoms selected from C, O, N and S in the ring, including at least one heteroatom, and typically one or two heteroatoms unless otherwise stated. The heteroatom or heteroatoms are typically selected from O, N, and S, most typically from S and N, especially N. For example, where the heterocyclyl ring is denoted a nitrogen-containing heterocyclyl group, it contains one nitrogen atom and optionally a further heteroatom selected from O, N and S. A heterocyclyl ring may be saturated or partially unsaturated, but is typically saturated. A 4- to 7-membered partially unsaturated heterocyclyl ring is a cyclic group containing from 4 to 7 atoms selected from C, O, N and S in the ring and containing 1 or 2, e.g. 1 double bond. Typically, in the compounds described herein, a heterocyclyl ring is saturated unless otherwise specified.

A 4- to 7-membered heterocyclyl ring may sometimes be a 5- to 6-membered ring. A 4- to 7-membered heterocyclyl ring is typically a monocyclic ring and may be a monocyclic 5- or 6-membered heterocyclyl ring. In some compounds described herein, a 4- to 7-membered heterocyclyl group is a 4- to 7-membered nitrogen-containing heterocyclyl ring which is unsubstituted or is substituted as described herein. Preferred 4- to 7-membered nitrogen-containing heterocyclyl rings include morpholine, pyrrolidine, piperidine and piperazine.

Examples of 5- and 6-membered saturated heterocyclyl rings include piperazine, piperidine, morpholine, diazinane and pyrrolidine. Diazinane is typically 1,4-diazinane.

As used herein, a 6- to 10-membered aryl ring is a substituted or unsubstituted, monocyclic or fused polycyclic aromatic group containing from 6 to 10 carbon atoms in the ring portion. Examples include monocyclic groups such as phenyl and fused bicyclic groups such as naphthyl and indenyl. Phenyl (benzene) is preferred.

As used herein, a 5- to 10-membered heteroaryl ring is a substituted or unsubstituted monocyclic or fused polycyclic aromatic group containing from 5 to 10 atoms in the ring portion, including at least one heteroatom, for example 1, 2 or 3 heteroatoms, typically selected from O, S and N. A heteroaryl ring is typically a 5- or 6-membered heteroaryl ring or an 8- to 10-membered heteroaryl ring. Preferably, the heteroaryl ring comprises 1, 2 or 3, preferably 1 or 2 nitrogen atoms.

Examples of 5- and 6-membered heteroaryl rings include thiazole, pyrazole, pyrimidine, triazole, 1,2,4-oxadiazole and pyrazine.

Examples of 8-, 9- and 10-membered heteroaryl rings include indazole, thieno[2,3-c]pyrazole, imidazo[4,5-b]pyridine, pyrazolo[3,4-b]pyridine, furo[2,3-c]pyridine, indole, benzoxazole, benzothiazole, [1,2,4]triazolo[4,3-a]pyridine, thieno[2,3-d]pyrimidine, 1,2,3-benzotriazole, imidazo[1,5-a]pyridine, imidazo[1,2-a]pyrazine, oxazolo[5,4-b]pyridine, quinoline, naphthyridine, isoquinoline, quinazoline, and quinoxaline. 8-, 9- and 10-membered heteroaryl rings as used herein are typically fused bicyclic groups.

For the avoidance of doubt, references to a heteroaryl ring also include fused polycyclic ring systems, including for instance fused bicyclic systems in which a heteroaryl ring is fused to an aryl group. When the heteroaryl ring is such a fused heteroaryl group, preferred examples are fused ring systems wherein a 5- to 6-membered heteroaryl group is fused to a phenyl group. Indazole is preferred.

As used herein, a fused bicyclic group is a group comprising two cyclic moieties sharing a common bond between two atoms.

When R⁸ is selected from a 6-membered aryl ring, a 5- to 6-membered heteroaryl ring, a 5- to 6-membered heterocyclyl ring and a 5- to 6-membered cycloalkyl ring, it is to be understood that R⁸ is a monocyclic ring.

A cycloalkyl, heterocyclyl, aryl or heteroaryl ring may be unsubstituted or substituted as described herein unless otherwise stated. For example, a cycloalkyl, heterocyclyl, aryl or heteroaryl ring may be unsubstituted or substituted with 1, 2 or 3, typically 1 or 2 such as e.g. 1 substituent. Suitable substituents include halo, C₁₋₄ alkoxy, R¹⁰, —(C₁₋₄ alkylene)-R¹⁰, ═O, —CN, and C₁₋₄ alkyl, wherein R¹⁰ is as defined herein). The substituents on a substituted cycloalkyl, heterocyclyl, aryl or heteroaryl ring are typically themselves unsubstituted, unless otherwise stated.

The compounds described herein comprise at least one heterocyclyl ring comprising at least one nitrogen atom. Said nitrogen atom(s) are independently selected from secondary, tertiary and quaternary nitrogen atom(s). A quaternary nitrogen atom is present when the compound comprises a quaternised derivative of one or more monocyclic groups or fused bicyclic groups. As used herein, a quaternised derivative of a moiety such as a cyclic moiety is formed by bonding an additional alkyl group to a nitrogen atom in the moiety such that the valency of the said nitrogen atom increases from 3 to 4 and the nitrogen atom is positively charged.

The compounds described herein comprise a heterocyclyl ring identified as ring Hy, which is a 4- to 7-membered heterocyclyl ring. Ring Hy contains X and at least one N atom in the ring portion, wherein X is a bond, —N(R¹¹)—, O, S, —S(O)₂—, —S(O)(NR¹¹)—, or —C(R¹¹)₂—, and R¹¹ is as defined herein. Ring Hy is linked to ring A (as identified in formula (I) above) via a C atom within ring Hy, said C atom also being linked to R² which is as defined herein. A N atom within ring Hy is substituted by R¹ which is as defined herein.

The rest of ring Hy is unsubstituted or substituted by one or two R³. Each R³ is independently selected from C₁₋₄ alkyl, C₁₋₄ alkoxy and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring or a C₃₋₆ heterocycloalkyl ring, as defined herein. In option (ii), the skilled person would understand that the C atom to which the two R³ are attached is a spiro atom (i.e. the common atom that connects the two rings of a spiro compound).

As used herein, the terms “monovalent” or “monovalent moiety” are used to describe a chemical group obtainable by removing a hydrogen atom from the corresponding compound. As used herein, the terms “divalent” or “divalent moiety” are used to describe a chemical group obtainable by removing a hydrogen atom from the corresponding monovalent moiety. Thus, as used herein, the terms “divalent” and “divalent moiety” are used to describe a chemical group obtainable by removing two hydrogen atoms from the corresponding compound.

It is to be understood that each individual atom present in the formulae depicted herein may be present in the form of any of its naturally occurring isotopes, with the most abundant isotope(s) being preferred. Thus, by way of example, each individual hydrogen atom present in the formulae depicted herein may be present as a 1H, 2H (deuterium) or 3H (tritium) atom, preferably 1H. Similarly, by way of example, each individual carbon atom present in the formulae depicted herein may be present as a 12C, 13C or 14C atom, preferably 12C.

In the compounds of the invention, the stereochemistry is not limited. In particular, where moiety M of formula (I) and/or moiety U contains one or more chiral centre, the compounds may be used in enantiomerically or diastereoisomerically pure form, or in the form of a mixture of isomers. Further, for the avoidance of doubt, the compounds of the invention may be used in any tautomeric form. Typically, the agent or substance described herein contains at least 50%, preferably at least 60%, 75%, 90% or 95% of a compound which is enantiomerically or diasteriomerically pure. Thus, the compound is preferably substantially optically pure.

The moiety M of formula (I) typically contains at least one chiral centre at the carbon atom of ring Hy which is linked to R² and to ring A. Moiety M may be provided in the form of the R-enantiomer at said carbon atom, in the form of the S-enantiomer at said carbon atom, or in the form of a mixture of the two enantiomers. The substance or agent described herein may contain at least 50%, preferably at least 60, 75%, 90% or 95% of a compound which is enantiomerically or diasteriomerically pure at moiety M. A pure enantiomeric form of either the R- or the S-enantiomer may be preferred. In some embodiments, the R-enantiomer at said carbon atom is preferred, and in particular when R² is H then the R-enantiomer at said carbon atom is preferred. The preferred stereochemistry at said carbon atom is also depicted by the following illustrative structure of formula (I) where the bond from ring Hy to the core ring A is in the “up” position and the bond from ring Hy to R² is the down position:

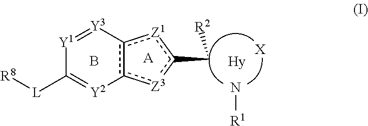

This steroechemsitry at the carbon atom of ring Hy which is linked to R² and to ring A, where the bond from ring Hy to the core ring A is in the “up” position and the bond from ring Hy to R² is the down position (as depicted above), is also preferred for the moiety M of formulae (II), (III), (IV), and for the compounds of formulae (I′), (II′) and (III′).

As discussed further herein, where the compounds of the invention contain a chiral centre, and in particular where moiety M and/or moiety U contains a chiral centre, the individual stereoisomers may be obtained by chiral synthesis, or by separation of stereoisomers. Stereoisomers may be separated, for example, using chiral chromatography. The individual stereoisomers may be identified using the IUPAC labels R and S as appropriate. Alternatively, the individual stereoisomers may be identified by the order in which they elute, for example the first, second, third or fourth stereisomer respectively, as obtained by separation using chiral chromatography. Where two or more stereocentres are present in a compound or moiety, a combination of IUPAC nomenclature for stereocentres where absolute stereochemistry has been defined, and rate of elution, may be used.

For example, where two or more individual stereoisomers are unidentified by IUPAC nomenclature, these stereoisomers may be identified as the first- and second-eluting isomers, as obtained by separation using chiral chromatography. Therefore, the stereoisomers at a chiral carbon atom of ring Hy which is linked to R² and to ring A may be defined as the first-eluting and second-eluting stereoisomers, as obtained by separation using chiral chromatography. Chiral chromatography is typically reverse phase HPLC.

The compounds of the present invention may exist in unsolvated as well as solvated forms with pharmaceutically acceptable solvents such as water, ethanol, and the like, and it is intended that the invention embrace both solvated and unsolvated forms.

A compound of the present invention can be converted into a pharmaceutically acceptable salt thereof, and a salt can be converted into the free compound, by conventional methods. For instance, a compound of the present invention can be contacted with a pharmaceutically acceptable acid to form a pharmaceutically acceptable salt. A pharmaceutically acceptable salt is a salt with a pharmaceutically acceptable acid or base.

Pharmaceutically acceptable acids include both inorganic acids such as hydrochloric, sulphuric, phosphoric, diphosphoric, hydrobromic or nitric acid and organic acids such as oxalic, citric, fumaric, maleic, malic, ascorbic, succinic, tartaric, benzoic, acetic, methanesulphonic, ethanesulphonic, benzenesulphonic orp-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines. Hydrochloride salts and acetate salts are preferred, in particular hydrochloride salts.

### Proteolysis Targeting Chimera (PROTAC)s

The compounds of the invention comprise a binder of MLLT1 and/or MLLT3 (M) that is covalently attached via a linker moiety (LINK) to an E3 ubiquitin ligase binding moiety (U). The inventors have surpisingly discovered that such compounds may have substantially enhanced efficacy compared with a corresponding MLLT1 and/or MLLT3 inhibitor that is not attached to an E3 ubiquitin ligase binding moiety. For example, the antiproliferative effects of the compounds may be substantially enhanced by covalent attachment to the E3 ubiquitin ligase binding moiety. Such compounds may induce selective degradation of MLLT1 and/or MLLT3. For instance, such compounds may induce selective degradation of MLLT1 and/or MLLT3 without inducing degradation of other proteins, such as FLT3 (fins like tyrosine kinase 3). Such compounds may also not inhibit FLT3.

Thus, the invention provides a compound which is a Proteolysis Targeting Chimera (PROTAC) or a pharmaceutically acceptable salt thereof, wherein the PROTAC has the structure:

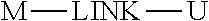

wherein U is an E3 ubiquitin ligase binding moiety, LINK is a moiety that covalently links M and U, and M is an MLLT1 and/or MLLT3 binder of formula (I) as defined herein.

In the compounds of the present invention, the MLLT1 and/or MLLT3 binder is covalently attached to the E3 ubiquitin ligase binding moiety via the moiety labelled LINK. LINK is either a single bond or a chemical group that covalently attaches the MLLT1 and/or MLLT3 binder to the E3 ubiquitin ligase binding moiety.

The MLLT1 and/or MLLT3 binder is attached to LINK via (i) a single bond at R⁸, or (ii) a C or N atom within group R⁸ or ring Hy (as described herein) such that a hydrogen atom on the C or N atom within group R⁸ or ring Hy is replaced with a bond to LINK. In case (ii), a hydrogen atom on the C or N atom within group R⁸ or ring Hy is replaced by either a direct single bond to the E3 ubiquitin ligase binding moiety or by a chemical group that covalently attaches the MLLT1 and/or MLLT3 binder to the E3 ubiquitin ligase binding moiety.

The description herein discusses the substitution of group R⁸ and ring Hy by various chemical groups aside from LINK. For completeness, the skilled person would readily appreciate that where group R⁸ and/or ring Hy are defined as being unsubstituted, this is intended as a reference to the absence of substituents other than a bond to LINK. Therefore, such a definition retains the option that group R⁸ or ring Hy is attached to LINK via a C or N atom within group R⁸ or ring Hy such that a hydrogen atom on the C or N atom within group R⁸ or ring Hy is replaced with a bond to LINK. Furthermore, the skilled person would readily appreciate that where group R⁸ and/or ring Hy are defined as being substituted by one or more chemical groups (suitable such chemical groups are defined herein), this is intended as a reference to substituents other than a bond to LINK. Therefore, such a definition includes group R⁸ or ring Hy being attached to LINK via a C or N atom within group R⁸ or ring Hy such that a hydrogen atom on the C or N atom within group R⁸ or ring Hy is replaced with a bond to LINK. Therefore, where group R⁸ and/or ring Hy are defined as being unsubstituted or substituted by one or more chemical groups (suitable such chemical groups are defined herein), then one of group R⁸ or ring Hy may also be attached to LINK via a C or N atom within group R⁸ or ring Hy such that a hydrogen atom on the C or N atom within group R⁸ or ring Hy is replaced with a bond to LINK. A bond to LINK may optionally be positioned on a carbon atom which carries a further substituent (where chemically possible). Typically, a carbon or nitrogen atom which is bonded to LINK does not carry a further substituent.

For the avoidance of doubt, where group R⁸ and/or ring Hy are defined as being substituted by a hydrogen atom (e.g. where R¹, R², R¹¹, R^{3a}, R^{3b}, R^{3c} and R^{3d} are defined as being a hydrogen atom), then a reference herein to a hydrogen atom within group R⁸ or ring Hy being replaced with a bond to LINK, includes the option that said hydrogen atom (e.g. a hydrogen atom at R¹, R², R¹¹, R^{3a}, R^{3b}, R^{3c} and R^{3d}) is replaced with a bond to LINK. Therefore, where R¹, R², R¹¹, R^{3a}, R^{3b}, R^{3c} and R^{3d} are defined as being a hydrogen atom, then said hydrogen atom may be replaced with a bond to LINK.

For the avoidance of doubt, where ring Hy or group R⁸ is substituted, a C or N atom which is bonded to LINK may be within the aryl, heteroaryl, heterocyclyl, or cycloalkyl ring of ring Hy or group R⁸, or it may be a C or N atom of a substituent on ring Hy or group R⁸. Preferably, a C or N atom which is bonded to LINK is a C or N atom on the heterocyclyl ring of ring Hy (i.e. directly to the ring and not via a substituent), a C or N atom of the heteroaryl ring of group R⁸, or the heteroaryl, cycloalkyl, heterocyclyl or phenyl ring of R¹⁰ in the case that a group R¹⁰ is present as, or as part of, a substituent on R⁸.

As the skilled person would readily appreciate, methods for preparation of compounds (in which two discrete chemical entities contribute discrete biological functions to the overall compound) are very well known in the art. A hugely diverse range of techniques for covalently attaching the respective chemical entities, via a vast number of chemical linker moieties, is well established. Such is the ubiquity of these methodologies, that standard text books devoted entirely to this topic have long been available. One such textbook is “Bioconjugate Techniques” (Greg T. Hermanson, Academic Press Inc., 1996), the content of which is herein incorporated by reference in its entirety.

MLLT1 and/or MLLT3 Binder Group M

In the compounds of the invention, the MLLT1 and/or MLLT3 binder M is a monovalent moiety of formula (I) as defined herein.

In the description that follows, the skilled person will readily understand that M is a monovalent moiety that is bonded to LINK via (i) a single bond at R⁸, or (ii) a C or N atom within group R⁸ or ring Hy (as described herein) such that a hydrogen atom on the C or N atom within group R⁸ or ring Hy is replaced with a bond to LINK. Thus, in the description that follows, any of the hydrogen atoms on any of the C or N atoms within group R⁸ or ring Hy may be removed to form the point of attachment of the monovalent moiety M to LINK.

Typically, ring Hy is a 5- to 6-membered heterocyclyl ring containing X and at least one N atom, for instance one or two N atoms. X is typically a bond, —N(R¹¹)—, O or —C(R¹¹)₂—. Preferably, X is a bond, —N(Me)-, O or —CH₂—, most preferably X is a bond. In particular, when ring Hy is a 4- or 5-membered heterocyclyl ring, X is most preferably a bond. For example, ring Hy may be selected from a pyrrolidinyl, piperidinyl, morpholinyl or diazinanyl ring. Preferably, ring Hy is a 5-membered heterocyclyl ring containing X and at least one N atom, for instance one N atom. For example, ring Hy may be a pyrrolidinyl ring.

Ring Hy is linked to ring A via a C atom within ring Hy, said C atom also being linked to R². A N atom within ring Hy is substituted by R¹. Typically, the N atom of ring Hy which is substituted by R¹ is adjacent to the C atom of ring Hy that is bonded to ring A.

A C or N atom within ring Hy may be bonded to LINK by a hydrogen atom on that C or N atom being replaced by a bond to LINK. Typically, where ring Hy is bonded to LINK, this is via a C atom on Hy. Preferably, the bond to LINK is via replacement of a hydrogen atom on a C or N within group R⁸, i.e. the bond to LINK is preferably not via ring Hy.

Typically, R¹ is H, C₁₋₄ cycloalkyl, or C₁₋₄ alkyl which is itself unsubstituted or substituted with one C₁₋₄ alkoxy or one, two or three halo, preferably with one C₁₋₄ alkoxy, for example R¹ may be H or C₁₋₂ alkyl which is unsubstituted or substituted with one C₁₋₂ alkoxy or one, two or three halo, preferably with one C₁₋₂ alkoxy, or R¹ may be H or C₁₋₂ alkyl which is unsubstituted or substituted with one C₁₋₂ alkoxy. Preferably, R¹ is H, methyl, ethyl or methoxyethyl, more preferably methyl or ethyl. Most preferably, R¹ is methyl.

R² is H or methyl. Preferably, R² is H.

Typically, each R¹¹ is independently selected from H and methyl. When X is —N(R¹¹)— or —S(O)(NR¹¹)—, R¹¹ is preferably methyl. When X is —C(R¹¹)₂—, optionally one R¹¹ is replaced with a single bond to LINK and the other R¹¹ is H. When X is —C(R¹¹)₂—, each R¹¹ is preferably H.

Aside from groups R¹ and R², ring Hy may further be unsubstituted or substituted by one or two R³. Typically, each R³ is independently selected from C₁₋₄ alkyl, C₁₋₄ alkoxy and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring. More typically, each R³ is independently selected from methyl, ethyl, t-butyl methoxy and fluoro, or (i) two R³ linked to adjacent C atoms in ring Hy form, together with the C atoms to which they are attached, a cyclohexyl ring, or (ii) two R³ linked to the same C atom in ring Hy form, together with the C atom to which they are attached, a cyclopentyl ring. Preferably, ring Hy is substituted by no R³ groups.

In another typical embodiment, each R³ is independently selected from methyl, ethyl, t-butyl, methoxy and fluoro, or (i) two R³ linked to adjacent C atoms in ring Hy form, together with the C atoms to which they are attached, a cyclohexyl ring, or (ii) two R³ linked to the same C atom in ring Hy form, together with the C atom to which they are attached, a cyclopropyl ring or a cyclopentyl ring. Preferably, ring Hy is substituted by two R³ groups which are both C₁₋₄ alkyl, for instance both being methyl, or one R³ group which is C₁₋₄ alkyl, for instance methyl, ethyl or t-butyl.

Typically, therefore, ring Hy is a 5- to 6-membered heterocyclyl ring containing X and at least one N atom, for example a pyrrolidinyl, piperidinyl, morpholinyl or diazinanyl ring; the N atom of ring Hy which is substituted by R¹ is adjacent to the C atom of ring Hy that is bonded to ring A; R¹ is H or C₁₋₂alkyl which is unsubstituted or substituted with one C₁₋₂ alkoxy or one, two or three halo, preferably with one C₁₋₂ alkoxy; R² is H or methyl; X is a bond, —N(R¹¹)—, O or —C(R¹¹)₂—; R¹¹ is H or methyl; ring Hy is further unsubstituted or substituted by one or two R³. and each R³ is independently selected from C₁₋₄ alkyl, C₁₋₄ alkoxy and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring.

Preferably, ring Hy is a pyrrolidinyl ring; the N atom of ring Hy which is substituted by R¹ is adjacent to the C atom of ring Hy that is bonded to ring A; R¹ is methyl or ethyl, more preferably methyl; R² is H; and ring Hy is substituted by no R³ groups, i.e. ring Hy is not further substituted and carries substituent R¹ only. In this embodiment, ring Hy may be referred to as being an unsubstituted pyrrolidine ring, wherein the skilled person would understand that ring Hy still carries substituent R¹. Alternatively, ring Hy is a pyrrolidinyl ring; the N atom of ring Hy which is substituted by R¹ is adjacent to the C atom of ring Hy that is bonded to ring A; R¹ is H, methyl, ethyl or methoxyethyl, more preferably methyl; R² is H; and ring Hy is further substituted by two R³ groups which are both C₁₋₄ alkyl, for instance both being methyl, or one R³ group which is C₁₋₄ alkyl, for instance methyl, ethyl or t-butyl.

Typically, no more than two of Y¹, Y² and Y³ are N. Preferably Y¹ is N, Y² is —C(R⁶)—, and Y³ is —C(R⁵)—.

Typically, Z¹ is —C(R⁴)—, Z³ is —N(H)—, and Y¹ is N. Thus, the bicyclic structure formed by rings A and B has the structure:

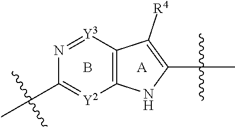

In one preferred embodiment, Z¹ is —C(R⁴)—, Z³ is —N(H)—, Y¹ is N, Y² is —C(R⁶)—, and Y³ is —C(R⁵)—, such that the bicyclic structure formed by rings A and B has the structure:

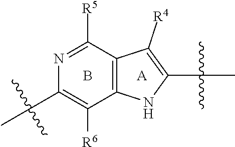

Typically, R⁴ is selected from H, CN, halo, C₁₋₄ alkoxy, and C₁₋₄ alkyl which is itself unsubstituted or substituted by one, two or three halo; and R⁵ and R⁶ are independently selected from H, halo, C₁₋₄ alkoxy, and C₁₋₄ alkyl which is itself unsubstituted or substituted by one, two or three halo. For instance, R⁴ is selected from H, halo (e.g. fluoro), methoxy, and methyl; and R⁵ and R⁶ are independently selected from H, halo (e.g. fluoro), methoxy, and methyl. Preferably, either R⁴, R⁵ and R⁶ are H, or one of R⁴, R⁵ and R⁶ is not H. More preferably, R⁴, R⁵ and R⁶ are H.

In one preferred embodiment, R⁵ and R⁶ are as defined herein and R⁴ is selected from H, halo, CN, C₁₋₄ alkoxy, and C₁₋₄ alkyl which is itself unsubstituted or substituted by one, two or three halo. More preferably, R⁵ and R⁶ are as defined herein and R⁴ is selected from H, halo, CN, C₁₋₄ alkyl and CF₃. Most preferably, R⁵ and R⁶ are as defined herein and R⁴ is selected from H, F, Cl, CN, Me and CF₃.

In one preferred embodiment, R⁵ is selected from H, cyclopropyl, C₁₋₄ alkoxy, and C₁₋₄ alkyl, for example from H, C₁₋₄ alkoxy, and C₁₋₄ alkyl. More preferably, R⁵ is selected from H, cyclopropyl, methoxy and methyl, for example H, methoxy and methyl.

In one preferred embodiment, R⁶ is H.

Typically, therefore, in formula (I):
R⁴ is selected from H, halo, CN, C₁₋₄ alkoxy, and C₁₋₄ alkyl which is itself unsubstituted or substituted by one, two or three halo, and R⁵ and R⁶ when present are H; or
R⁵ is selected from H, cyclopropyl, C₁₋₄ alkoxy, and C₁₋₄ alkyl, typically from H, C₁₋₄ alkoxy, and C₁₋₄ alkyl, and R⁴ and R⁶ when present are H; or
R⁴, R⁵ and R⁶ are H.

Preferably, in formula (I):
R⁴ is selected from H, F, Cl, Me, CN and CF₃, and R⁵ and R⁶ when present are H;
R⁵ is selected from H, cyclopropyl, methoxy and methyl, typically from H, methoxy and methyl, and R⁴ and R⁶ when present are H; or
R⁴, R⁵ and R⁶ are H.

In one alternative preferred embodiment, R⁵ and R⁶ are as defined herein and R⁴ is selected from halo, CN, C₁₋₄ alkoxy, and C₁₋₄ alkyl which is itself unsubstituted or substituted by one, two or three halo. More preferably, R⁵ and R⁶ are as defined herein and R⁴ is selected from halo, CN, C₁₋₄ alkyl and CF₃. More preferably, R⁵ and R⁶ are as defined herein and R⁴ is selected from halo, and CF₃. Most preferably, R⁵ and R⁶ are as defined herein and R⁴ is selected from F, Cl, and CF₃.

L is —C(O)N(H)—, wherein the C atom of L is bonded to R⁸, and the N atom of L is bonded to ring B.

In one embodiment, R⁸ is a bond to LINK.

In another embodiment, R⁸ is a group selected from a 6-membered aryl ring, a 5- to 6-membered heteroaryl ring, a 5- to 6-membered heterocyclyl ring, and a 5- to 6-membered cycloalkyl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹. R⁹ and R¹⁰ are as defined herein.

Typically, R⁸ is a group selected from a 6-membered aryl ring, a 5- to 6-membered heteroaryl ring, and a 5- to 6-membered heterocyclyl ring. The aryl, heteroaryl and heterocyclyl rings are as defined herein. For instance, R⁸ may be selected from phenyl, thiazolyl, pyrazolyl, pyrimidinyl, pyridinyl, triazolyl, 1,2,4-oxadiazolyl, and pyrazinyl. Preferably, R⁸ is phenyl or a 5- to 6-membered heteroaryl ring preferably containing one, two or three N atoms. More preferably, R⁸ is a phenyl group, a pyridinyl, a pyrimidinyl or a pyrazolyl group. In particular, R⁸ may be a phenyl group.

A C or N atom within group R⁸ may be bonded to LINK by a hydrogen atom on that C or N atom being replaced by a bond to LINK. Typically, where group R⁸ is bonded to LINK, this is via a C atom on R. Preferably, a C or N atom within group R⁸ is bonded to LINK such that a hydrogen atom on that C or N atom is replaced by a bond to LINK. Preferably, the C or N atom that is bonded to LINK is a C or N atom of the aryl, heteroaryl, heterocyclyl or cycloalkyl ring of R⁸ or a C or N atom of the heteroaryl, cycloalkyl, heterocyclyl or phenyl ring of R¹⁰. More preferably, the C or N atom that is bonded to LINK is a C or N atom of the aryl, heteroaryl, heterocyclyl or cycloalkyl ring of R.

In addition to any bond to LINK (where present) R⁸ is unsubstituted or substituted by one, two or three substituents, for instance one or two substituents. Typically, each substituent is independently selected from halo, C₁₋₄ alkoxy, R¹⁰, —(CH₂)—R¹⁰, ═O, —CN, and C₁₋₄ alkyl which is itself unsubstituted or substituted by one or two groups selected from selected from halo and C₁₋₂ alkoxy. More typically, each substituent is independently selected from fluoro, chloro, methoxy, R¹⁰, —(CH₂)—R¹⁰, ═O, —CN, and C₁₋₂ alkyl which is itself unsubstituted or substituted by one or two groups selected from selected from halo (e.g. fluoro) and methoxy. Preferably, each substituent is independently selected from fluoro, R¹⁰ and methyl. Most preferably, each substituent is independently selected from fluoro and methyl. R¹⁰ is as defined herein.

Typically, R¹⁰ is a group selected from a 5- to 6-membered heteroaryl ring, a 3- to 6-membered cycloalkyl ring, a 4- to 5-membered heterocyclyl ring, and a phenyl ring. The cycloalkyl, heteroaryl and heterocyclyl rings are as defined herein. Preferably, R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl, more preferably pyrazolyl.

Typically, in addition to any bond to LINK (where present), R¹⁰ is unsubstituted or substituted by one or two substituents independently selected from fluoro, methyl and methoxy. Preferably, R¹⁰ is unsubstituted.

Typically, therefore, R⁸ is (i) a bond to LINK or (ii) a group selected from a 6-membered aryl ring, a 5- to 6-membered heteroaryl ring, a 5- to 6-membered heterocyclyl ring, and a 5- to 6-membered cycloalkyl ring; the group R⁸ is unsubstituted or substituted by one, two or three substituents, for instance one or two substituents, each independently selected from halo, C₁₋₄ alkoxy, R¹⁰, —(CH₂)—R¹⁰, ═O, —CN, and C₁₋₄ alkyl which is itself unsubstituted or substituted by one or two groups selected from selected from halo and C₁₋₂alkoxy; wherein R¹⁰ is a group selected from a 5- to 6-membered heteroaryl ring, a 3- to 6-membered cycloalkyl ring, a 4- to 5-membered heterocyclyl ring, and a phenyl ring; and R¹⁰ is unsubstituted or substituted by one or two substituents independently selected from fluoro, methyl and methoxy. Typically, where a C or N atom within group R⁸ is bonded to LINK, the C or N atom that is bonded to LINK is a C or N atom of the aryl, heteroaryl, heterocyclyl or cycloalkyl ring of R⁸ or a C or N atom of the heteroaryl, cycloalkyl, heterocyclyl or phenyl ring of R¹⁰.

Preferably, R⁸ is phenyl or a 5- to 10-membered heteroaryl ring containing one, two or three N atoms, for example a phenyl group or a pyrazolyl group, a pyrimidinyl group or a pyridinyl group; the group R⁸ is unsubstituted or substituted by one or two substituents, each independently selected from fluoro, R¹⁰ and methyl; R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl; and R¹⁰ is unsubstituted.

Preferably, a C or N atom within group R⁸ is bonded to LINK, wherein the C or N atom that is bonded to LINK is a C or N atom of the aryl, heteroaryl, heterocyclyl or cycloalkyl ring of R⁸.

In a typical embodiment of formula (I), the bicyclic structure formed by rings A and B has the structure:

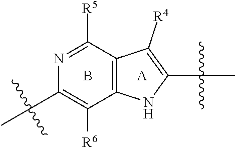

and:
ring Hy is a 5- to 6-membered heterocyclyl ring containing X and at least one N atom, for example a pyrrolidinyl, piperidinyl, morpholinyl or diazinanyl ring; wherein the N atom of ring Hy which is substituted by R¹ is adjacent to the C atom of ring Hy that is bonded to ring A;
R¹ is H or C₁₋₂ alkyl which is unsubstituted or substituted with one C₁₋₂ alkoxy or one, two or three halo, preferably with one C₁₋₂ alkoxy;
R² is H or methyl;
X is a bond, —N(Me)-, O or —CH₂—;
ring Hy is further unsubstituted or substituted by one or two R³; wherein each R³ is independently selected from C₁₋₄ alkyl, C₁₋₄ alkoxy and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring;
R⁴ is selected from H, CN, halo (e.g. fluoro or chloro), methoxy, methyl and trifluoromethyl;
R⁵ and R⁶ are independently selected from H, halo (e.g. fluoro or chloro), methoxy, methyl and trifluoromethyl;
the C atom of L is bonded to R⁸, and the N atom of L is bonded to ring B;
R⁸ is (i) a bond to LINK or (ii) a group selected from a 6-membered aryl ring, a 5- to 6-membered heteroaryl ring, a 5- to 6-membered heterocyclyl ring, and a 5- to 6-membered cycloalkyl ring; wherein the group R⁸ is unsubstituted or substituted by one, two or three substituents, for instance one or two substituents, each independently selected from halo, C₁₋₄ alkoxy, R¹⁰, —(CH₂)—R¹⁰, ═O, —CN, and C₁₋₄ alkyl which is itself unsubstituted or substituted by one or two groups selected from halo and C₁₋₂ alkoxy; and
R¹⁰ is a group selected from a 5- to 6-membered heteroaryl ring, a 3- to 6-membered cycloalkyl ring, a 4- to 5-membered heterocyclyl ring, and a phenyl ring; wherein R¹⁰ is unsubstituted or substituted by one or two substituents independently selected from fluoro, methyl and methoxy;
wherein either R⁸ is (i) a bond to LINK, or R⁸ is a group (ii) and M is bonded to LINK via a C or N atom within group R⁸ or ring Hy such that a hydrogen atom on the C or N atom within group R⁸ or ring Hy is replaced with a bond to LINK.

In a preferred embodiment of formula (I), the bicyclic structure formed by rings A and B has the structure:

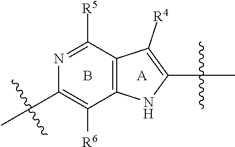

and:
ring Hy is a pyrrolidinyl ring; wherein the N atom of ring Hy which is substituted by R¹ is adjacent to the C atom of ring Hy that is bonded to ring A; R¹ is H, methyl, ethyl or methoxyethyl, more preferably methyl; R² is H; and ring Hy is substituted by no R³ groups; either R⁴, R⁵ and R⁶ are H, or one of R⁴, R⁵ and R⁶ is defined as described above except that it is not H, and the rest are H;
the C atom of L is bonded to R⁸, and the N atom of L is bonded to ring B;
R⁸ is phenyl or a 5- to 6-membered heteroaryl ring containing one, two or three N atoms; the group R⁸ is unsubstituted or substituted by one or two substituents, each independently selected from fluoro, R¹⁰ and methyl; R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl; and R¹⁰ is unsubstituted;
wherein either R⁸ is (i) a bond to LINK, or R⁸ is a group (ii) and M is bonded to LINK via a C or N atom within group R⁸ or ring Hy such that a hydrogen atom on the C or N atom within group R⁸ or ring Hy is replaced with a bond to LINK.

In a particular embodiment of the compounds of the invention, the MLLT1 and/or MLLT3 binder group M is a monovalent moiety of formula (II):

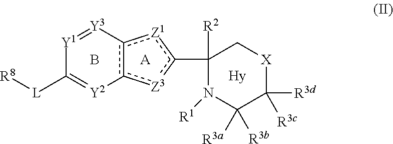

wherein:
Z¹, Z³, Y¹, Y², Y³, R¹, R², R⁸, X and L are as defined herein;
R^{3a} and R^{3b} are independently selected from H, C₁₋₄ alkyl, phenyl, a 5- to 6-membered heteroaryl ring and C₁₋₄ alkoxy, or R^{3a} and R^{3b} form, together with the C atom to which they are attached, a C₃₋₆ cycloalkyl ring or a C₃₋₆ heterocycloalkyl ring,
R^{3c} and R^{3d} are independently selected from H, C₁₋₄ alkyl, halo, phenyl, a 5- to 6-membered heteroaryl ring and C₁₋₄ alkoxy, or R^{3c} and R^{3d} form, together with the C atom to which they are attached, a C₃₋₆ cycloalkyl ring or a C₃₋₆ heterocycloalkyl ring, or
R^{3a} and R^{3c} are H, and R^{3b} and R^{3d} form, together with the C atoms to which they are attached, a C₅₋₆ cycloalkyl ring, with the proviso that when X is —N(R¹¹)—, O, S, —S(O)₂— or —S(O)(NR¹¹)-then neither R^{3c} nor R^{3d} are halo;
wherein at least two of R^{3a}, R^{3b}, R^{3c} and R^{3d} are H; and
wherein either R⁸ is (i) a bond to LINK, or R⁸ is a group (ii) and M is bonded to LINK via a C or N atom within group R⁸ or ring Hy such that a hydrogen atom on the C or N atom within group R⁸ or ring Hy is replaced with a bond to LINK.

Typically, in formula (II): R^{3a} and R^{3b} are independently selected from H and methyl; R^{3c} and R^{3d} are independently selected from H, C₁₋₄ alkyl, halo and C₁₋₄ alkoxy, in particular from H, methyl, ethyl, fluoro, methoxy and t-butyl, or R^{3c} and R^{3d} form, together with the C atom to which they are attached, a C₅₋₆ cycloalkyl ring (in particular a cyclopentyl ring), with the proviso that when X is —N(R¹¹)—, O, S, —S(O)₂— or —S(O)(NR¹¹)—, then neither R^{3c} nor R^{3d} are fluoro; or R^{3a} and R^{3c} are H, and R^{3b} and R^{3d} form, together with the C atoms to which they are attached, a C₅₋₆ cycloalkyl ring (in particular a cyclohexyl ring); wherein at least two of R^{3a}, R^{3b}R^{3c} and R^{3d} are H. In an alternative typical embodiment: R^{3a} and R^{3b} are independently selected from H and methyl; R^{3c} and R^{3d} are independently selected from H, C₁₋₄ alkyl, halo and C₁₋₄ alkoxy, in particular from H, methyl, ethyl, fluoro, methoxy and t-butyl, or R^{3c} and R^{3d} form, together with the C atom to which they are attached, a C₃₋₆ cycloalkyl ring (in particular a cyclopropyl or cyclopentyl ring), with the proviso that when X is NR¹¹, O, or S, then neither R^{3c} nor R^{3d} are fluoro; or R^{3a} and R^{3c} are H, and R^{3b} and R^{3d} form, together with the C atoms to which they are attached, a C₅₋₆ cycloalkyl ring (in particular a cyclohexyl ring); wherein at least two of R^{3a}, R^{3b}, R^{3c} and R^{3d} are H.

Optionally, one of R^{3a}, R^{3b}, R^{3c} and R^{3d} is replaced with a single bond to LINK.

Preferably, in formula (II), R^{3a}, R^{3b}, R^{3c} and R^{3d} are all H, or one of R^{3a}, R^{3b}, R^{3c} and R^{3d} is methyl, ethyl or t-butyl and the rest are H, or R^{3c} and R^{3d} are methyl and the rest are H. More preferably, R^{3a}, R^{3b}, R^{3c} and R^{3d} are all H.

In a typical embodiment of formula (II), the bicyclic structure formed by rings A and B has the structure:

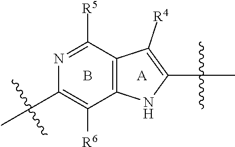

and:
R¹ is H or C₁₋₂ alkyl which is unsubstituted or substituted with one C₁₋₂ alkoxy or one, two or three halo, preferably with one C₁₋₂ alkoxy;
R² is H or methyl;
X is a bond, —N(Me)-, O or —CH₂—;
R^{3a} and R^{3b} are independently selected from H and methyl; R^{3c} and R^{3d} are independently selected from H, C₁₋₄ alkyl, halo and C₁₋₄ alkoxy, in particular from H, methyl, ethyl, fluoro, methoxy and t-butyl, or R^{3c} and R^{3d} form, together with the C atom to which they are attached, a C₅₋₆ cycloalkyl ring (in particular a cyclopentyl ring), with the proviso that when X is NR¹¹ or O, then neither R^{3c} nor R^{3d} are fluoro; or R^{3a} and R^{3c} are H, and R^{3b} and R^{3d} form, together with the C atoms to which they are attached, a C₅₋₆ cycloalkyl ring (in particular a cyclohexyl ring);
wherein at least two of R^{3a}, R^{3b}, R^{3c} and R^{3d} are H;
R⁴ is selected from H, CN, halo (e.g. fluoro or chloro), methoxy, methyl and trifluoromethyl;
R⁵ and R⁶ are independently selected from H, halo (e.g. fluoro or chloro), methoxy, methyl and trifluoromethyl;
the C atom of L is bonded to R⁸, and the N atom of L is bonded to ring B;
R⁸ is (i) a bond to LINK or (ii) a group selected from a 6-membered aryl ring, a 5- to 6-membered heteroaryl ring, a 5- to 6-membered heterocyclyl ring, and a 5- to 6-membered cycloalkyl ring; wherein the group R⁸ is unsubstituted or substituted by one, two or three substituents, for instance one or two substituents, each independently selected from halo, C₁₋₄ alkoxy, R¹⁰, —(CH₂)—R¹⁰, ═O, —CN, and C₁₋₄ alkyl which is itself unsubstituted or substituted by one or two groups selected from selected from halo and C₁₋₂alkoxy; and
R¹⁰ is a group selected from a 5- to 6-membered heteroaryl ring, a 3- to 6-membered cycloalkyl ring, a 4- to 5-membered heterocyclyl ring, and a phenyl ring; wherein R¹⁰ is unsubstituted or substituted by one or two substituents independently selected from fluoro, methyl and methoxy;
wherein either R⁸ is (i) a bond to LINK, or R⁸ is a group (ii) and M is bonded to LINK via a C or N atom within group R⁸ or ring Hy such that a hydrogen atom on the C or N atom within group R⁸ or ring Hy is replaced with a bond to LINK.

In a preferred embodiment of formula (II), the bicyclic structure formed by rings A and B has the structure:

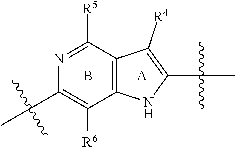

and:
X is absent;
R¹ is H, methyl, ethyl or methoxyethyl, more preferably methyl;
R² is H;
R^{3a}, R^{3b}, R^{3c} and R^{3d} are all H;
either R⁴, R⁵, and R⁶, are H, or one of R⁴, R⁵, and R⁶ is selected from CN, fluoro, methoxy, methyl and trifluoromethyl, and the rest are H, with the proviso that R⁵, and R⁶ cannot be CN; the C atom of L is bonded to R⁸, and the N atom of L is bonded to ring B;
R⁸ is phenyl or a 5- to 6-membered heteroaryl ring containing one, two or three N atoms, for example an indazolyl group; the group R⁸ is unsubstituted or substituted by one or two substituents, each independently selected from fluoro, R¹⁰ and methyl; R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl; and R¹⁰ is unsubstituted; and
wherein either R⁸ is (i) a bond to LINK, or R⁸ is a group (ii) and M is bonded to LINK via a C or N atom within group R⁸ or ring Hy such that a hydrogen atom on the C or N atom within group R⁸ or ring Hy is replaced with a bond to LINK.

In a particularly preferred embodiment of the compounds of the invention, the MLLT1 and/or MLLT3 binder M is a monovalent moiety of formula (III):

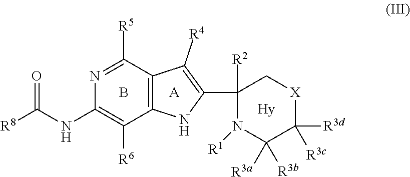

wherein R¹, R², R^{3a}, R^{3b}, R^{3c}, R^{3a}, X, R⁴, R⁵, R⁶ and R⁸ are as defined herein.

Typically, in the compounds of formula (III):
R¹ is H or C₁₋₄ alkyl which is itself unsubstituted or substituted with C₁₋₄ alkoxy;
R² is H or methyl;
R^{3a} and R^{3b} are independently selected from H, C₁₋₄ alkyl, and C₁₋₄ alkoxy, or R^{3a} and R^{3b} form, together with the C atom to which they are attached, a C₃₋₆ cycloalkyl ring,
R^{3c} and R^{3d} are independently selected from H, C₁₋₄ alkyl, halo and C₁₋₄ alkoxy, or R^{3c} and R^{3d} form, together with the C atom to which they are attached, a C₃₋₆ cycloalkyl ring, or
R^{3a} and R^{3c} are H, and R^{3b} and R^{3d} form, together with the C atoms to which they are attached, a C₅₋₆ cycloalkyl ring, with the proviso that when X is —N(Me)- or O, then neither R^{3c} nor R^{3d} are halo;
wherein at least two of R^{3a}, R^{3b}, R^{3c} and R^{3d} are H;
X is a bond, —N(Me)-, O or —CH₂—;
R⁴ is selected from H, halo C₁₋₄ alkoxy, and C₁₋₄ alkyl which is itself unsubstituted or substituted by one, two or three halo;
R⁵ and R⁶ are independently selected from H, halo C₁₋₄ alkoxy, and C₁₋₄ alkyl which is itself unsubstituted or substituted by one, two or three halo;
R⁸ is (i) a bond to LINK or (ii) a group selected from a 6-membered aryl ring, a 5- to 6-membered heteroaryl ring, a 5- to 6-membered heterocyclyl ring, and a 5- to 6-membered cycloalkyl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy; and
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo;
wherein either R⁸ is (i) a bond to LINK, or R⁸ is a group (ii) and M is bonded to LINK via a C or N atom within group R⁸ or ring Hy (as described herein) such that a hydrogen atom on the C or N atom within group R⁸ or ring Hy is replaced with a bond to LINK.

Typically, in formula (III), R¹ is H, or C₁₋₄ alkyl which is itself unsubstituted or substituted with one C₁₋₄ alkoxy or one, two or three halo, preferably with one C₁₋₄ alkoxy, for example R¹ may be H or C₁₋₂ alkyl which is unsubstituted or substituted with one C₁₋₂ alkoxy or one, two or three halo, preferably with one C₁₋₂ alkoxy. Preferably, R¹ is H, methyl, ethyl or methoxyethyl. Most preferably, R¹ is methyl.

In formula (III), R² is H or methyl. Preferably, R² is H.

Typically, in formula (III), X is a bond, —N(R¹¹)—, O or —C(R¹¹)₂—. Preferably, X is a bond, —N(Me)-, O or —CH₂—, most preferably X is a bond.

Typically, in formula (III), each R¹¹ is independently selected from H and methyl. When X is —N(R¹¹)—, R¹¹ is preferably methyl. When X is —C(R¹¹)₂—, one R¹¹ is optionally replaced with a single bond to LINK and the other R¹¹ is H. When X is —C(R¹¹)₂—, each R¹¹ is preferably H.

Typically, in formula (III), R^{3a} and R^{3b} are independently selected from H, C₁₋₄ alkyl, halo and C₁₋₄ alkoxy, in particular from H and methyl; R^{3c} and R^{3d} are independently selected from H, methyl, ethyl, fluoro, methoxy and t-butyl, or R^{3c} and R^{3d} form, together with the C atom to which they are attached, a C₅₋₆ cycloalkyl ring (in particular a cyclopentyl ring), with the proviso that when X is NR¹¹ or O, then neither R^{3c} nor R^{3d} are fluoro; or R^{3a} and R^{3c} are H, and R^{3b} and R^{3d} form, together with the C atoms to which they are attached, a C₅₋₆ cycloalkyl ring (in particular a cyclohexyl ring); wherein at least two of R^{3a}, R^{3b}, R^{3c} and R^{3d} are H.

Preferably, in formula (III), R^{3a}, R^{3b}, R^{3c} and R^{3d} are all H. Alternatively, one of R^{3a}, R^{3b}, R^{3c} and R^{3d} is methyl, ethyl or t-butyl, and the rest are H, or R^{3c} and R^{3d} are methyl and the rest are H.

In all embodiments of formula (III) described herein, when X is —N(R¹¹)— or O then neither R^{3c} nor R^{3d} are halo (e.g. fluoro).

Typically, in formula (III), R⁴ is selected from H, CN, halo, C₁₋₄ alkoxy, and C₁₋₄ alkyl which is itself unsubstituted or substituted by one, two or three halo. Preferably, R⁴ is selected from H, CN, F, C₁, Me and CF₃.

Typically, in formula (III), R⁵ is selected from H and C₁₋₄ alkyl. Preferably, R⁵ is selected from H and methyl, more preferably H.

Typically, in formula (III), R⁶ is H.

Typically, therefore, in formula (III):
R⁴ is selected from H, CN, halo, C₁₋₄ alkoxy, and C₁₋₄ alkyl which is itself unsubstituted or substituted by one, two or three halo, and R⁵ and R⁶ are H; or
R⁵ is selected from H and C₁₋₄ alkyl, and R⁴ and R⁶ when present are H; or
R⁴, R⁵, and R⁶ when present are H.

Preferably, in in formula (III):
R⁴ is selected from selected from H, CN, F, Cl, Me and CF₃, and R⁵ and R⁶ are H; or
R⁵ is selected from H and methyl, and R⁴ and R⁶ when present are H; or
R⁴, R⁵, and R⁶ when present are H.

In one alternative preferred embodiment, R⁵ and R⁶ are as defined herein and R⁴ is selected from halo, CN, C₁₋₄ alkoxy, and C₁₋₄ alkyl which is itself unsubstituted or substituted by one, two or three halo. More preferably, R⁵ and R⁶ are as defined herein and R⁴ is selected from halo, CN, C₁₋₄ alkyl and CF₃. More preferably, R⁵ and R⁶ are as defined herein and R⁴ is selected from halo, and CF₃. Most preferably, R⁵ and R⁶ are as defined herein and R⁴ is selected from F, Cl, and CF₃.

In one preferred embodiment of formula (III), R⁸ is a group selected from phenyl, pyridinyl, thiazolyl, pyrazolyl, pyrimidinyl, triazolyl, 1,2,4-oxadiazolyl, and pyrazinyl. Preferably, R⁸ is phenyl or a 5- to 6-membered heteroaryl ring containing one, two or three N atoms.

Typically, in formula (III), R⁸ is unsubstituted or substituted by one, two or three substituents, for instance one or two substituents, independently selected from halo, C₁₋₄ alkoxy, R¹⁰, —(CH₂)—R¹⁰, ═O, —CN, and C₁₋₄ alkyl which is itself unsubstituted or substituted by one or two groups selected from selected from halo and C₁₋₂ alkoxy. More typically, each substituent is independently selected from fluoro, chloro, methoxy, R¹⁰, —(CH₂)—R¹⁰, ═O, —CN, and C₁₋₂ alkyl which is itself unsubstituted or substituted by one or two groups selected from selected from halo (e.g. fluoro) and methoxy.

Preferably, each substituent is independently selected from fluoro, R¹⁰ and methyl. Most preferably, each substituent is independently selected from fluoro and methyl. R¹⁰ is as defined herein.

Typically, in formula (III), R¹⁰ is a group selected from a 5- to 6-membered heteroaryl ring, a 3-to 6-membered cycloalkyl ring, a 4- to 5-membered heterocyclyl ring, and a phenyl ring. The aryl, heteroaryl and heterocyclyl rings are as defined herein. Preferably, R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl, more preferably pyrazolyl.

Typically, in in formula (III), R¹⁰ is unsubstituted or substituted by one or two substituents independently selected from fluoro, methyl and methoxy. Preferably, R¹⁰ is unsubstituted.

In typical embodiments of formula (III):
R¹ is H or C₁₋₂ alkyl which is unsubstituted or substituted with one C₁₋₂ alkoxy or one, two or three halo, preferably with one C₁₋₂ alkoxy;
R² is H or methyl;
X is a bond, —N(Me)-O or —C(H)₂—;
R^{3a} and R^{3b} are independently selected from H and methyl; R^{3c} and R^{3d} are independently selected from H, C₁₋₄ alkyl, halo and C₁₋₄ alkoxy, in particular from H, methyl, ethyl, fluoro, methoxy and t-butyl, or R^{3c} and R^{3d} form, together with the C atom to which they are attached, a C₅₋₆ cycloalkyl ring (in particular a cyclopentyl ring), with the proviso that when X is NR¹¹ or O, then neither R^{3c} nor R^{3d} are fluoro; or R^{3a} and R^{3c} are H, and R^{3b} and R^{3d} form, together with the C atoms to which they are attached, a C₅₋₆ cycloalkyl ring (in particular a cyclohexyl ring);
wherein at least two of R^{3a}, R^{3b}, R^{3c} and R^{3d} are H;
one of the following options applies: R⁴ is selected from H, CN, halo, C₁₋₄ alkoxy, and C₁₋₄ alkyl which is itself unsubstituted or substituted by one, two or three halo, and R⁵ and R⁶ are H; or R⁵ is selected from H and C₁₋₄ alkyl, and R⁴ and R⁶ when present are H; or
R⁴, R⁵, and R⁶ when present are H;
the group R⁸ is unsubstituted or substituted by one, two or three substituents, for instance one or two substituents, each independently selected from halo, C₁₋₄ alkoxy, R¹⁰, —(CH₂)—R¹⁰, ═O, —CN, and C₁₋₄ alkyl which is itself unsubstituted or substituted by one or two groups selected from selected from halo and C₁₋₂ alkoxy; and
R¹⁰ is a group selected from a 5- to 6-membered heteroaryl ring, a 3- to 6-membered cycloalkyl ring, a 4- to 5-membered heterocyclyl ring, and a phenyl ring; wherein R¹⁰ is unsubstituted or substituted by one or two substituents independently selected from fluoro, methyl and methoxy; and R⁸ is a a 6-membered aryl ring, or a 5- to 6-membered heteroaryl ring;
wherein either R⁸ is (i) a bond to LINK, or R⁸ is a group (ii) and M is bonded to LINK via a C or N atom within group R⁸ or ring Hy such that a hydrogen atom on the C or N atom within group R⁸ or ring Hy is replaced with a bond to LINK.

In preferred embodiments of formula (III),
X is absent;
R¹ is H, methyl, ethyl or methoxyethyl, more preferably methyl;
R² is H;
R^{3a}, R^{3b}, R^{3c} and R^{3d} are all H;
R⁵ and R⁶ are H, and R⁴ is selected from H, CN, F, C₁, Me and CF₃, preferably H, CN or Cl, more preferably H;
R⁸ is phenyl or a 5- to 6-membered heteroaryl ring containing one, two or three N atoms; the group R⁸ is unsubstituted or substituted by one or two substituents, each independently selected from fluoro, R¹⁰ and methyl;
R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl, preferably pyrazolyl; and R¹⁰ is unsubstituted;
wherein either R⁸ is (i) a bond to LINK, or R⁸ is a group (ii) and M is bonded to LINK via a C or N atom within group R⁸ or ring Hy such that a hydrogen atom on the C or N atom within group R⁸ or ring Hy is replaced with a bond to LINK.

In a most preferred embodiment of the compounds of the invention, the MLLT1 and/or MLLT3 binder M is a monovalent moiety of formula (IV):

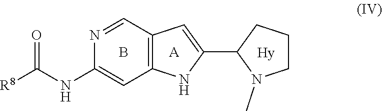

wherein R⁸ is as defined herein. In particular in formula (IV), the group R⁸ may be unsubstituted or substituted by one, two or three substituents independently selected from R¹⁰, halo and C₁₋₄ alkyl;
wherein either R⁸ is (i) a bond to LINK, or R⁸ is a group (ii) and M is bonded to LINK via a C or N atom within group R⁸ such that a hydrogen atom on the C or N atom within group R⁸ is replaced with a bond to LINK.

Typically, in formula (IV), R⁸ is a group selected from phenyl, thiazolyl, pyrazolyl, pyrimidinyl, triazolyl, 1,2,4-oxadiazolyl, pyridinyl and pyrazinyl.

Typically, in formula (IV), R⁸ is unsubstituted or substituted by one, two or three substituents, for instance one or two substituents, independently selected from halo and C₁₋₄ alkyl and/or by one group R¹⁰, wherein R¹⁰ is preferably pyrazolyl; preferably R⁸ is unsubstituted or substituted by one, two or three substituents, for instance one or two substituents, independently selected from halo and C₁₋₄ alkyl. Preferably, each substituent is independently selected from fluoro and methyl.

In a particularly preferred embodiment, in formula (IV), R⁸ is selected from one of the following structures:

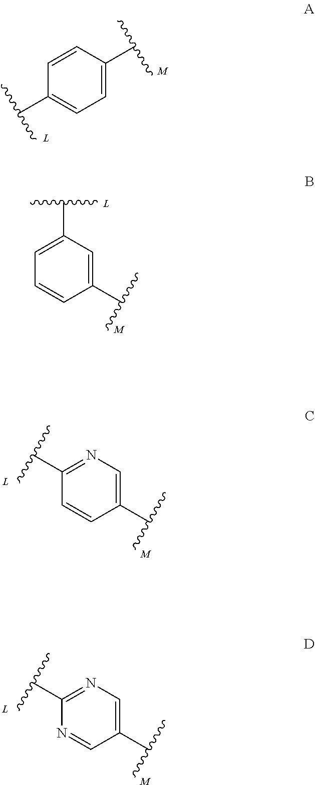

wherein:

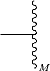

represents the point of attachment of R⁸ to the rest of moiety M; and

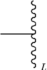

represents the point of attachment of R⁸ to LINK or R¹⁰.

When

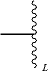

represents the point of attachment to R¹⁰, then R¹⁰ is attached to LINK.

For the avoidance of doubt, the group R⁸ is further unsubstituted or substituted as described above. Preferably, the group R⁸ is further unsubstituted or substituted by one or two substituents independently selected from fluorine and chlorine. For example, the group R⁸ may be selected from one of the following structures:

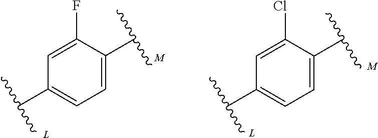

wherein:

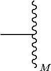

represents the point of attachment of R⁸ to the rest of moiety M; and

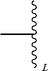

represents the point of attachment of R⁸ to LINK.

For the avoidance of doubt, R⁸ may be selected from any one of the above structures defined herein in any of formulae (I), (II) and (III).

In preferred embodiments of the compounds of the invention, the MLLT1 and/or MLLT3 binder M is a monovalent moiety:

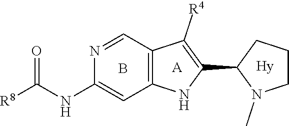

wherein R⁴ and R⁸ are as defined herein.

In some of these preferred embodiments, R⁴ is selected from H and halo (such as flouro or chloro). In one such embodiment, R⁴ is H. In another such embodiment, R⁴ is halo (such as flouro or chloro).

In a particularly preferred embodiment, the MLLT1 and/or MLLT3 binder M is a monovalent moiety:

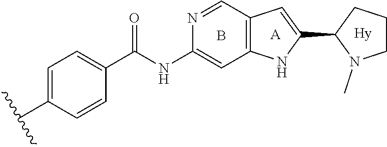

### E3 Ubiquitin Ligase Binding Moieties

The presence of the E3 ubiquitin ligase binding moiety U in the compound of the invention means that the compound is a so-called “PROTAC”.

The term PROTAC is an acronym for proteolysis targeting chimera. In general, PROTACs are, as is known in the art, heterobifunctional molecules that comprise two active moieties attached covalently by a linker group. In a PROTAC, the first active moiety (the MLLT1 and/or MLLT3 binder M in the compound of the present invention) binds to a target protein that is intended for degradation (target proteins for the compound of the present invention are MLLT1 and/or MLLT3). The second active moiety (U in the compound of the present invention) is capable of binding to an E3 ubiquitin ligase, thereby inducing selective intracellular proteolysis. Recruitment of the E3 ligase to the target protein results in ubiquitination and subsequent degradation of the target protein by the proteasome.

E3 ubiquitin ligase moities are known in the art. Substantially any such moiety can be used. The sole limitation on the moiety is that it be capable of binding to an E3 ubiquitin ligase. Those skilled in the art would appreciate that entirely routine laboratory methods can be used to determine whether a given substance binds to an E3 ubiquitin ligase (including but not limited to any of those disclosed specifically herein). Thus, those skilled in the art would have no difficulty in identifying E3 ubiquitin ligase moieties, nor in establishing whether any existing chemical moiety falls within the bounds of this definition. In certain embodiments, the moiety shows activity or binds to the E3 ubiquitin ligase with an IC₅₀ of less than about 200 mM. The IC₅₀ can be determined according to any method known in the art, e.g., a fluorescent polarization assay.

Merely by way of example of the extensive disclosure in the field concerning PROTACs, and hence E3 ubiquitin ligase binding moieties, reference can be made to Gu et al. (BioEssays 2018, 40, 1700247), Sun et al. (Signal Transduction and Targeted Therapy (2019) 4:64), WO 2020/041331, and WO 2019/140003, the contents of all of which are herein incorporated by reference in their entireties. Any of the numerous E3 ubiquitin ligase binding moieties disclosed in these documents can be used as an E3 ubiquitin ligase binding moiety in the compounds of the present invention. For the avoidance of doubt, Gu et al. refer to such moieties as ligands to recruit E3 ubiquitin ligase, Sun et al. refer to such moieties as E3 ubiquitin ligase (E3) recruiting ligands, in WO 2020/041331 such moieties are referred to as a “ULM” or (small molecule) E3 ubiquitin ligase binding moiety (that binds an E3 ubiquitin ligase) (and noting that the term “ULM” includes each of “ILM”, “CLM”, “VLM” and “MLM”, any of which can be used in the present compounds), and in WO 2019/140003 such moieties (labelled “B” in WO 2019/140003's formula (I)) are referred to as a ubiquitin ligase ligand/binder.

Further examples of documents disclosing suitable moieties that can be used in the compounds of the present invention are WO2013/106643, US2016/0045607, WO2014187777, US20140356322 and U.S. Pat. No. 9,249,153, US2016/0058872, US2015/0291562 and Winter et al (Science, Jun. 19, 2015, p. 1376), the contents of all of which are herein incorporated by reference in their entireties. Thalidomide, lenalidomide, pomalidomide and analogs thereof are still further examples of suitable moieties.

Examples of E3 ubiquitin ligases include von Hippel-Lindau (VHL) and cereblon (CRBN).

Preferably the E3 ubiquitin ligase binding moiety (U) is capable of binding to CRBN or VHL.

Such moieties are referred to herein as CRBN or VHL E3 ubiquitin ligase binding moieties.

In one embodiment, U is a CRBN E3 ubiquitin ligase binding moiety. In another embodiment, U is a VHL E3 ubiquitin ligase binding moiety.

In some embodiments, the E3 ubiquitin ligase binding moiety (U) is:
(a) a CRBN E3 ubiquitin ligase binding moiety of formula (U1):

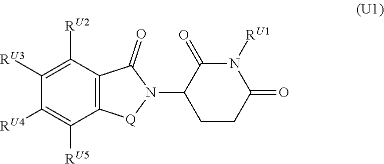

wherein:
R^{U1} is H; Q is selected from —CH(R^{U6})—, —N(R^{U6})—, —O—, —C(O)—, —NH—CH(R^{U6})—,
—N═C(R^{U6})—, or —N═N—;
R^{U6} is H or C₁₋₄alkyl;
R^{U2} and R^{U5} are each independently selected from H, halogen, C₁₋₄alkyl, NH₂, NO₂, OH, COOH, CN, CF₃, and a single bond to LINK;
R^{U3} and R^{U4} are each independently selected from H, halogen, C₁₋₄alkyl, NH₂, NO₂, OH, COOH, CN, CF₃, and a single bond to LINK;
wherein one and only one of R^{U2}, R^{U3}, R^{U4} and R^{U5} is a single bond to LINK;
(b) a CRBN E3 ubiquitin ligase binding moiety of formula (U4):

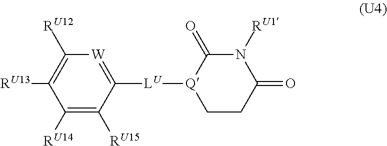

wherein:
R^{U1′} is H;
W is N or CR^{U16};
Q′ is N and L^{U} is a single bond, or Q′ is CH and L^{U} is selected from a single bond or —C(O)N(H)—, wherein either (i) the C atom of L^{U} is bonded to phenyl, and the N atom of L^{U} is bonded to Q′; or (ii) the C atom of L^{U} is bonded to Q′, and the N atom of L^{U} is bonded to phenyl;
R^{U12}, R^{U13} and R^{U14} are each independently selected from H, halogen, C₁₋₄ alkyl, NH₂, NO₂, OH, COOH, CN, CF₃, and a single bond to LINK;
wherein one and only one of R^{U2}, R^{U13} and R^{U14} is a single bond to LINK;
R^{U15} and R^{U16} are each independently selected from H, halogen, C₁₋₄ alkyl, NH₂, NO₂, OH, COOH, CN and CF₃;
(c) a CRBN E3 ubiquitin ligase binding moiety of formula (U5):

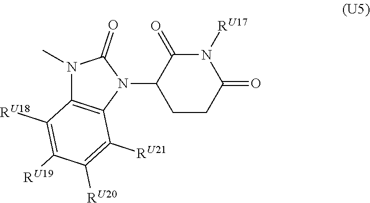

wherein:
R^{U17} is H;
R^{U18}, R^{U19}, R^{U2O} and R^{U21} are each independently selected from H, halogen, C₁₋₄ alkyl, NH₂, NO₂, OH, COOH, CN, CF₃, and a single bond to LINK;
wherein one and only one of R^{U18}, R^{U19}, R^{U20} and R^{U21} is a single bond to LINK;
or (d) a VHL E3 ubiquitin ligase binding moiety of formula (U2):

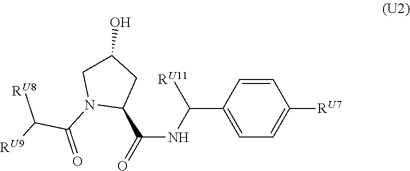

wherein:
R^{U7} is a group selected from phenyl, a 5- to 6-membered heteroaryl ring, a 5- to 6-membered heterocyclyl ring, and a 5- to 6-membered cycloalkyl ring, the group R^{U7} being unsubstituted or substituted by C₁₋₄ alkyl;
R^{U11} is H or C₁₋₄ alkyl;
R^{U8} is selected from C₁₋₄ alkyl, phenyl and a 3- to 8-membered cycloalkyl ring; and
R^{U9} is a single bond to LINK.

For the avoidance of doubt, in formula (U1), when Q is —NH—CH(R^{U6})— or —N═C(R^{U6})—, the N atom of Q is bonded to the phenyl ring in U, and the C atom of Q (not including any C atom that may be in R^{U6}) is bonded to the N atom in U that is adjacent to group Q.

In some embodiments, the E3 ubiquitin ligase binding moiety (U) is preferably a CRBN E3 ubiquitin ligase binding moiety of formula (U1).

Typically, in formula (U1), Q is selected from —CH(R^{U6})— and —C(O)—. Preferably, Q is selected from —CH₂— and —C(O)—. Most preferably, Q is —CH₂—.

Typically, in formula (U1), R^{U6} is H or methyl. Preferably, R^{U6} is H.

Typically, in formula (U1), R^{U2} and R^{U5} are each independently selected from H, fluoro, methyl, NH₂, NO₂, OH, COOH, CN, CF₃, and a single bond to LINK. Preferably, R^{U2} and R^{U5} are each independently selected from H and a single bond to LINK. Most preferably, R^{U2} is H, and R^{U5} is a single bond to LINK.

Typically, in formula (U1), R^{U3} and R^{U4} are each independently selected from H, fluoro, methyl, NH₂, NO₂, OH, COOH, CN, CF₃ and a single bond to LINK. Preferably, R^{U3} and R^{U4} are each independently selected from H and a single bond to LINK.

Typically, in formula (U1), two or three of R^{U2}, R^{U3}, R^{U4} and R^{U5} are H. Preferably, three of R^{U2}, R^{U3}, R^{U4} and R^{U5} are H. More preferably, R^{U2} and R^{U3} are H, one of R^{U4} and R^{U5} is a single bond to LINK, and the other of R^{U4} and R^{U5} is H. Most preferably, R^{U2}, R^{U3} and R^{U4} are H, and R^{U5} is a single bond to LINK.

Typically, therefore, in formula (U1):
Q is selected from —CH(R^{U6})— and —C(O)—;
R^{U6} is H or methyl;
R^{U2} and R^{U5} are each independently selected from H, fluoro, methyl, NH₂, NO₂, OH, COOH, CN, CF₃, and a single bond to LINK;
R^{U3} and R^{U4} are each independently selected from H, fluoro, methyl, NH₂, NO₂, OH, COOH, CN, CF₃ and a single bond to LINK; and
two or three of R^{U2}, R^{U3}, R^{U4} and R^{U5} are H.

Preferably, in formula (U1):
Q is selected from —CH₂— and —C(O)—;
R^{U6} is H;
one of R^{U2}, R^{U3}, R^{U4} and R^{U5} is a single bond to LINK, and the rest are H, for instance wherein R^{U2} and R^{U3} are H, one of R^{U4} and R^{U5} is a single bond to LINK, and the other of R^{U4} and R^{U5} is H.

Most preferably, in formula (U1):
Q is —CH₂—;
R^{U6} is H;
R^{U2}, R^{U3} and R^{U4} are H, and R^{U5} is a single bond to LINK.

Typically, in formula (U4), Q′ is N and L^{U} is a single bond, or Q′ is CH and L^{U} is —C(O)N(H)—. Preferably, Q′ is N and L^{U} is a single bond.

Typically, in formula (U4), when L^{U} is —C(O)N(H)—, then the C atom of L^{U} is bonded to phenyl, and the N atom of L^{U} is bonded to Q′.

Typically, in formula (U4), R^{U12}, R^{U13} and R^{U14} are each independently selected from H, fluoro, methyl, NH₂, NO₂, OH, COOH, CN, CF₃, and a single bond to LINK (wherein one and only one of R^{U2}, R^{U13} and R^{U14} is a single bond to LINK). Preferably, one of R^{U12}, R^{U13} and R^{U14} is a single bond to LINK, one of R^{U2}, R^{U13} and R^{U14} is H and the other of R^{U12}, R^{U13} and R^{U14} is selected from H, fluoro, methyl, NH₂, NO₂, OH, COOH, CN, CF₃, preferably from H, fluoro and methyl. More preferably, one of R^{U2}, R^{U13} and R^{U14} is a single bond to LINK and the other two are H, for instance wherein R^{U12} and R^{U14} are H, and R^{U13} is a single bond to LINK.

Typically, in formula (U4), R^{U15} and R^{U16} (when present) are each independently selected from H, fluoro, methyl, NH₂, NO₂, OH, COOH, CN and CF₃. Preferably, R^{U15} and R^{U16} (when present) are each independently selected from H, F and Me.

In one embodiment of formula (U4), R^{U15} and R^{U16} (when present) are H, and two of R^{U2}, R^{U13} and R^{U14} are H. Preferably, R^{U13}, R^{U14} and R^{U15} are H, R^{U12} is a single bond to LINK, and R^{U16} (when present) is selected from H, F and Me.

Typically, therefore, in formula (U4):
Q′ is N and L^{U} is a single bond, or Q′ is CH and L^{U} is —C(O)N(H)—; R^{U12}, R^{U13} and R^{U14} are each independently selected from H, fluoro, methyl, NH₂, NO₂, OH, COOH, CN, CF₃, and a single bond to LINK; and
R^{U15} and R^{U16} (when present) are each independently selected from H, fluoro, methyl, NH₂, NO₂, OH, COOH, CN and CF₃;
wherein one and only one of R^{U12}, R^{U13} and R^{U14} is a single bond to LINK.

Preferably, in formula (U4):
Q′ is N and L^{U} is a single bond;
one of R^{U12}, R^{U13} and R^{U14} is a single bond to LINK and the other two are H, for instance wherein R^{U12} and R^{U14} are H, and R^{U13} is a single bond to LINK, or R^{U13}, R^{U14} and R^{U15} are H, R^{U12} is a single bond to LINK, and R^{U16} (when present) is selected from H, F and Me.

Typically, in formula (U5), R^{U1}s, R^{U19}, R^{U20} and R^{U21} are each independently selected from H, fluoro, methyl, NH₂, NO₂, OH, COOH, CN, CF₃, and a single bond to LINK (wherein one and only one of R^{U18}, R^{U19}, R^{U20} and R^{U21} is a single bond to LINK). Preferably, one of R^{U18}, R^{U19}, R^{U20} and R^{U21} is a single bond to LINK, one or two, preferably two of R^{U18}, R^{U19}, R^{U20} and R^{U21} are H and the other one or two, preferably one of R^{U18}, R^{U19}, R^{U20} and R^{U21} are selected from H, fluoro, methyl, NH₂, NO₂, OH, COOH, CN, CF₃, preferably from H, fluoro and methyl. More preferably, one of R^{U18}, R^{U19}, R^{U20} and R^{U21} is a single bond to LINK and the other three are H, for instance wherein R^{U19}, R^{U20} and R^{U21} are H, and R^{U18} is a single bond to LINK.

Typically, in formula (U2), R^{U7} is a 5- to 6-membered heteroaryl ring. Preferably, R^{U7} is a 5-membered heteroaryl ring containing one S atom and optionally one N atom.

Typically, in formula (U2), R^{U7} is unsubstituted or substituted by C₁₋₄ alkyl. Preferably, R^{U7} is unsubstituted or substituted by methyl.

Typically, in formula (U2), R^{U11} is H or methyl. Preferably, R^{U11} is H.

Typically, in formula (U2), R^{U8} is C₁₋₄ alkyl or phenyl. Preferably, R^{U8} is t-butyl or phenyl, more preferably t-butyl.

Typically, therefore, in formula (U2), R^{U7} is a 5- to 6-membered heteroaryl ring, the group R^{U7} being unsubstituted or substituted by C₁₋₄ alkyl, R^{U11} is H or methyl, and R^{U8} is C₁₋₄ alkyl or phenyl.

Preferably, in formula (U2), R^{U7} is a 5-membered heteroaryl ring containing one S atom and optionally one N atom, the group R^{U7} being unsubstituted or substituted by methyl, R^{U11} is H, and R^{U8} is t-butyl or phenyl, more preferably t-butyl.

In a preferred embodiment, the E3 ubiquitin ligase binding moiety (U) is a VHL E3 ubiquitin ligase binding moiety of formula (U3):

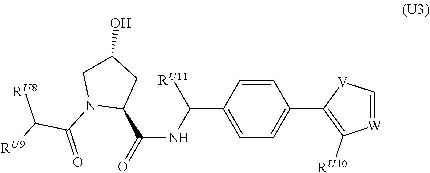

wherein:
V is S or O;
W is N or CH;
R^{U10} is H or C₁₋₄ alkyl;
R^{U11} is H or C₁₋₄ alkyl;
R^{U8} is selected from C₁₋₄ alkyl, phenyl and a 3- to 8-membered cycloalkyl ring; and
R^{U9} is a single bond to LINK.

Typically, in formula (U3), V is S.

Typically, in formula (U3), W is N or CH. Preferably, W is N.

Typically, in formula (U3), R^{U10} is H or methyl. Preferably, R^{U10} is methyl.

Typically, in formula (U3), R^{U11} is H or methyl. Preferably, R^{U11} is H.

Typically, in formula (U3), R^{U8} is C₁₋₄ alkyl or phenyl. Preferably, R^{U8} is t-butyl or phenyl, more preferably t-butyl.

Typically, therefore, in formula (U3), V is S, W is N or CH, R^{U10} is H or methyl, R^{U11} is H or methyl, and R^{U8} is C₁₋₄ alkyl or phenyl. Preferably, V is S, W is N, R^{U10} is methyl, R^{U11} is H, R^{U8} is t-butyl or phenyl, more preferably t-butyl.

The moieties U of formulae (U1), (U4) when Q′ is CH and (U5) typically contain at least one chiral centre at the carbon atoms marked with an asterisk as depicted below:

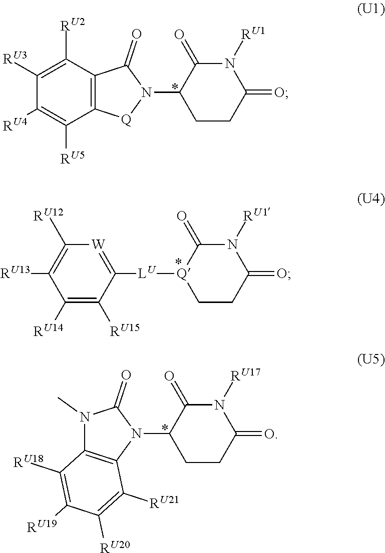

Moiety U may therefore be provided in the form of the R-enantiomer at said carbon atom, in the form of the S-enantiomer at said carbon atom, or in the form of a mixture (for example a 1:1 mixture) of the two enantiomers. A pure enantiomeric form of either the R- or the S-enantiomer may be preferred. In some embodiments, the S-enantiomer at said carbon atom is preferred. In other embodiments, the R-enantiomer at said carbon atom is preferred. The substance or agent described herein may contain at least 50%, preferably at least 60, 75%, 90% or 95% of a compound which is enantiomerically or diasteriomerically pure at moiety U. Unless otherwise stated, the moiety U may typically be provided in the form of a 1:1 mixture of the two enantiomers.

In some embodiments, the S-enantiomer at said carbon atom is preferred. These compounds have been shown to be the more active enantiomer in the literature. The preferred stereochemistry at said carbon atom is also depicted by the following illustrative structure of formula (U1):

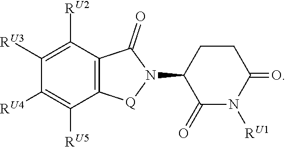

where LINK is attached to moiety U at the R^{U5} position.

In some preferred embodiments, the E3 ubiquitin ligase binding moiety (U) has the following structure:

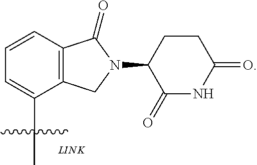 [SMILES: *c1cccc2c1CN([C@H]1CCC(=O)NC1=O)C2=O]

### Linkers

As discussed herein, in the compounds of the invention, the binder of MLLT1 and/or MLLT3 (M) is covalently attached to an E3 ubiquitin ligase binding moiety (U). This covalent attachment is labelled LINK and may be a direct single bond or a divalent chemical linker group that forms covalent bonds both to the binder of MLLT1 and/or MLLT3 (M), and to the E3 ubiquitin ligase binding moiety (U). Typically, LINK is a divalent chemical linker group that forms covalent bonds both to the binder of MLLT1 and/or MLLT3 (M), and to the E3 ubiquitin ligase binding moiety (U).

There is no particular limitation on the nature of LINK in the compounds of the present invention (beyond that the respective active moieties, e.g., U and M, are able to exert their desired function and LINK is capable of covalently attaching them together). Those skilled in the art would recognise that chemical linker groups are routinely used in the construction of bifunctional molecules and would be able routinely to provide appropriate chemical linker groups for attaching particular U and M moieties together. Typically, a chemical linker group for use in the present invention is an organic group.

When LINK is a divalent chemical linker group, it may be represented by formula (L):

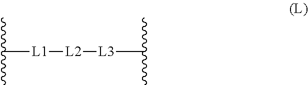

wherein:
LINK is attached to M via L1;
LINK is attached to U via L3;
L1 and L3 are each independently selected from a single bond, —N(R′)—, —C(O)N(R′)—, —O—, —N(R′)C(O)—, —C(O)—, —S(O₂)N(R′)—, —N(R′)S(O₂)—, —(C₁₋₆ alkylene)-, ethynylene, —(C₂₋₆ alkenylene)-, —C═C═C—, phenylene, a divalent 3- to 6-membered cycloalkyl ring and a divalent 4- to 7-membered heterocyclyl ring, the phenylene, cycloalkyl and heterocyclyl rings being unsubstituted or substituted by one or two C₁₋₄ alkyl;
L2 is represented by the formula -(L4)ₘ-;
each L4 is independently a unit of formula:

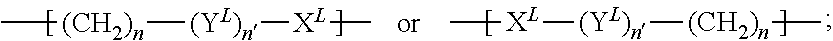

each X^{L} is independently selected from a single bond, —N(R′)—, —O—, —C(O)—, —S—, —SO—, —SO₂—, —C(R″)═C(R″)— and —C≡C—;
each Y^{L} is independently selected from a divalent 3- to 6-membered cycloalkyl ring, a divalent 4- to 7-membered heterocyclyl ring, or one of the following structures:

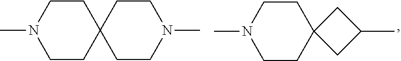

the cycloalkyl and heterocyclyl rings being unsubstituted or substituted by one or two C₁₋₄ alkyl;
n is selected from 0 to 4;
n′ is selected from 0 or 1;
n+n′≥1;
m is selected from 1 to 30;
each R′ is independently selected from H and C₁₋₄ alkyl; and
each R″ is independently selected from H and halo.

When m is not 1 (i.e. when there are two or more L4 groups present), then the X^{L} group of any L4 group is not directly bonded to the X^{L} group of any other L4 group that is present.

When the phenylene, cycloalkyl or heterocyclyl rings referred to herein are substituted, they are prefereably substituted by one methyl group.

In a preferred embodiment, L1 and L3 are each independently selected from a single bond, —C(O)N(R′)—, —O—, —N(R′)C(O)—, —C(O)—, —S(O₂)N(R′)—, —N(R′)S(O₂)—, —(C₁₋₆ alkylene)-, ethynylene, —(C₂₋₆ alkenylene)-, —C═C═C—, phenylene, a divalent 3- to 6-membered cycloalkyl ring and a divalent 4- to 7-membered heterocyclyl ring, the phenylene, cycloalkyl and heterocyclyl rings being unsubstituted or substituted by one methyl group.

Typically, in formula (L), L1 is selected from a single bond, branched or straight-chain —(C₁₋₆ alkylene)-, —C(O)N(R′)—, —N(R′)C(O)—, —O—, —S(O₂)N(R′)—, —N(R′)S(O₂)—, and a divalent 4- to 7-membered heterocyclyl ring which is unsubstituted or substituted by one or two C₁₋₄ alkyl. When present, the divalent 4- to 7-membered heterocyclyl ring of L1 typically contains at least one N atom, for instance one or two N atoms. Preferably, when L1 is a divalent 4- to 7-membered heterocyclyl ring, it is a divalent 5- to 6-membered heterocyclyl ring, more preferably a saturated divalent 5- to 6-membered heterocyclyl ring, for instance a divalent moiety of piperidine or diazinane. Preferably, in formula (L), L1 is selected from a single bond, branched or straight-chain —(C₁₋₆ alkylene)-, —C(O)N(H)—, —N(H)C(O)—, and a divalent moiety of piperidine or diazinane which is unsubstituted or substituted by one methyl group. For instance, L1 may be a single bond.

Typically, in formula (L), L3 is selected from a single bond, —N(R′)—, —C(O)N(R′)—, —O—, —N(R′)C(O)—, —S(O₂)N(R′)—, —N(R′)S(O₂)—, ethynylene, —C═C═C—, phenylene and a divalent 4- to 7-membered heterocyclyl ring, the phenylene and heterocyclyl rings being unsubstituted or substituted by one or two C₁₋₄ alkyl. When present, the divalent 4- to 7-membered heterocyclyl ring of L3 typically contains at least one N atom, for instance one or two N atoms. Preferably, when L3 is a divalent 4- to 7-membered heterocyclyl ring, it is a divalent 5- to 6-membered heterocyclyl ring, more preferably a saturated divalent 5- to 6-membered heterocyclyl ring, for instance a divalent moiety of piperidine or diazinane, preferably 1,4-diazinane. Preferably, in formula (L), L3 is selected from a single bond, —N(H)—, —C(O)N(H)—, —O—, —N(H)C(O)—, ethynylene, —C═C═C—, phenylene and a divalent moiety of piperidine or diazinane, the phenylene and divalent moiety of piperidine or diazinane being unsubstituted or substituted by one methyl group. Most preferably, L3 is selected from a single bond, ethynylene and a divalent moiety of piperidine or diazinane.

L2 is represented by the formula -(L4)ₘ-. In formula (L), each L4 is independently a unit of formula

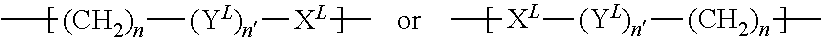

as described above. Typically, each X^{L} is independently selected from a single bond, —O—, —S—, —C(R″)═C(R″)— and —C≡C—, for example a single bond, —O— and —S—. Preferably, each X^{L} is independently selected from a single bond and —O—.

Typically, each Y^{L} is independently selected from a divalent moiety of piperidine or diazinane, or one of the following structures:

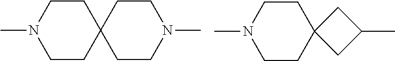

the divalent moiety of piperidine or diazinane being unsubstituted or substituted by one methyl group.

In formula (L), n is selected from 0 to 4; n′ is selected from 0 or 1; and n+n′≥1.

In one embodiment, each L4 is the same. Alternatively, L2 may comprise two or more blocks, wherein in each block, each L4 is the same. For instance, L2 may be represented by the formula -(L4′)ₚ-(L4″)_{q}-(L4″′)ᵣ-, wherein L4′, L4″ and L4″′ are selected from those moieties described above for L4, and wherein each L4′ is the same, each L4″ is the same and each L4″′ is the same. Preferably, L4′, L4″ and L4″′ are selected from —CH₂—, —CH₂CH₂O—, —OCH₂CH₂—, —CH₂CH₂S— and —SCH₂CH₂—. More preferably, L4′, L4″ and L4″′ are selected from —CH₂—, —CH₂CH₂O— and —OCH₂CH₂—. p, q and r are integers of from 0 to 30, wherein p+q+r=m.

In one embodiment, each L4 unit is the same. For instance, each L4 in formula (L) may be —CH₂—, each L4 in formula (L) may be —CH₂CH₂O—, each L4 in formula (L) may be —OCH₂CH₂—, each L4 in formula (L) may be —CH₂CH₂S—, or each L4 in formula (L) may be —SCH₂CH₂—. More preferably, each L4 in formula (L) is —CH₂—, each L4 in formula (L) is —CH₂CH₂O— or each L4 in formula (L) is —OCH₂CH₂—. Most preferably, each L4 in formula (L) is —CH₂—.

Typically, in formula (L), m is selected from 1 to 10. For instance, m may be selected from 1 to 8, 1 to 6, 1 to 5, 1 to 4, 1 to 3, 1 to 2, or 1.

Typically, in formula (L), each R′ is independently selected from H and methyl. Preferably, each R′ is H.

When L2 comprises one or more divalent 4- to 7-membered heterocyclyl rings, typically L2 comprises only one or two, e.g. one divalent 4- to 7-membered heterocyclyl rings. Typically, therefore, L2 comprises none, one or two divalent 4- to 7-membered heterocyclyl rings, for example none or one divalent 4- to 7-membered heterocyclyl rings. When L2 comprises one or more divalent 4- to 7-membered heterocyclyl rings, then in one embodiment L1 and L3 do not contain a divalent 4- to 7-membered heterocyclyl ring.

Typically, therefore, in formula (L):
L1 is selected from a single bond, branched or straight-chain —(C₁₋₆ alkylene)-, —C(O)N(R′)—, —N(R′)C(O)—, —O—, —S(O₂)N(R′)—, —N(R′)S(O₂)—, and a divalent 4- to 7-membered heterocyclyl ring which is unsubstituted or substituted by one or two C₁₋₄ alkyl, the divalent 4- to 7-membered heterocyclyl ring typically containing at least one N atom, for instance one or two N atoms;
L3 is selected from a single bond, —N(R′)—, —C(O)N(R′)—, —O—, —N(R′)C(O)—, —S(O₂)N(R′)—, —N(R′)S(O₂)—, ethynylene, —C═C═C—, phenylene and a divalent 4- to 7-membered heterocyclyl ring, the phenylene and heterocyclyl rings being unsubstituted or substituted by one or two C₁₋₄ alkyl, the divalent 4- to 7-membered heterocyclyl ring typically containing at least one N atom, for instance one or two N atoms; each X^{L} is independently selected from a single bond, —O—, —S—, —C(R″)═C(R″)— and —C≡C—, for example a single bond, —O— and —S—;
each Y^{L} is independently selected from a divalent moiety of piperidine or diazinane, or one of the following structures:

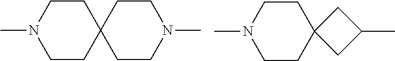

the divalent moiety of piperidine or diazinane being unsubstituted or substituted by one methyl group;
n is selected from 0 to 4; n′ is selected from 0 or 1; and n+n′≥1;
m is selected from 1 to 10, preferably from 1 to 4 (for example 1, 2 or 3);
each R′ is independently selected from H and methyl; and
each R″ is independently selected from H, fluro and chloro, for instance H and chloro.

Preferably, in formula (L):
L1 is selected from a single bond, branched or straight-chain —(C₁₋₆ alkylene)-, —C(O)N(H)—, —N(H)C(O)—, and a divalent moiety of piperidine or diazinane which is unsubstituted or substituted by one methyl group;
L3 is selected from a single bond, —C(O)N(H)—, —O—, —N(H)C(O)—, ethynylene, —C═C═C—, phenylene and a divalent moiety of piperidine or diazinane, the phenylene and divalent moiety of piperidine or diazinane being unsubstituted or substituted by one methyl group; each X^{L} is independently selected from a single bond and —O—;
each Y^{L} is independently selected from a divalent moiety of piperidine or diazinane, or one of the following structures:

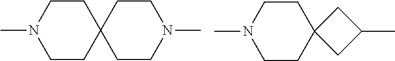

the divalent moiety of piperidine or diazinane being unsubstituted or substituted by one methyl group;
n is selected from 0 to 4; n′ is selected from 0 or 1; and n+n′≥1; m is selected from 1 to 4 (for example 1, 2 or 3);
each R′ is H; and
each R″ is H or chloro, for instance H.

In a typical embodiment, L2 is —(C₁₋₁₀ alkylene)- or —(C₁₋₁₀ alkylene)-C(O)—, preferably —(C₁₋₁₀ alkylene)-. In this embodiment, L1 is preferably selected from a single bond, —C(O)N(H)—, —N(H)C(O)—, —O—, and a divalent moiety of piperidine or diazinane. In this embodiment, L3 is preferably selected from a single bond, —C(O)N(H)—, —O—, —N(H)C(O)—, ethynylene and a divalent moiety of piperidine or diazinane. More preferably, L1 and L3 are selected from the following options:
L1 is —C(O)N(H)— or —N(H)C(O)—, and L3 is —C(O)N(H)— or —N(H)C(O)—;
L1 is a divalent moiety of piperidine and L3 is a divalent moiety of diazinane;
L1 is —C(O)N(H)— or —N(H)C(O)—, and L3 is a divalent moiety of diazinane;
L1 is a single bond and L3 is a divalent moiety of diazinane;
L1 is a single bond and L3 is a divalent moiety of piperidine;
L1 is a single bond and L3 is —O—;
L1 is a single bond and L3 is ethynylene;
L1 is a single bond and L3 is a single bond; and
L1 is —C(O)N(H)— or —N(H)C(O)—, and L3 is —O—.

In particularly preferred embodiments:
L1 is a divalent moiety of piperidine, L2 is —(C₁₋₁₀ alkylene)- and L3 is a divalent moiety of diazinane;
L1 is a single bond, L2 is —(C₁₋₁₀ alkylene)- and L3 is —O—;
L1 is a single bond, L2 is —(C₁₋₁₀ alkylene)- and L3 is a divalent moiety of piperidine or diazinane;
L1 is a single bond, L2 is —(C₁₋₁₀ alkylene)- and L3 is ethynylene;
L1 is a divalent moiety of piperidine, L2 is —(C₁₋₁₀ alkylene)- and L3 is ethynylene;
L1 is a divalent moiety of diazinane, L2 is —(C₁₋₁₀ alkylene)- and L3 is ethynylene;
L1 is a divalent moiety of diazinane, L2 is —(C₁₋₁₀ alkylene)- and L3 is a divalent moiety of piperidine;
L1 is a single bond, L2 is a divalent moiety of piperidine and L3 is phenylene;
L1 is a divalent moiety of piperidine or diazinane, L2 is —(C₁₋₁₀ alkylene)- and L3 is phenylene;
L1 is —O—, L2 is —(C₁₋₁₀ alkylene)- and L3 is ethynylene;
L1 is a divalent moiety of piperidine, L2 is —(C₁₋₁₀ alkylene)-O— and L3 is a divalent moiety of piperidine.

In one preferred embodiment, LINK has the following structure:

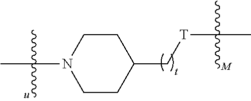

wherein T is —O— or —(CH₂)—, and t is selected from 1 to 10, preferably 1 to 5, more preferably 2.

In another preferred embodiment, L1 is a single bond, L2 is —(C₁₋₁₀ alkylene)- and L3 is ethynylene or a divalent moiety of piperidine (for instance ethynylene), preferably wherein L2 is —(C₃₋₈ alkylene)-, more preferably wherein L2 is —(C₇ alkylene)-.

Alternatively, when LINK is a divalent chemical linker group, it may be represented by formula (L′):

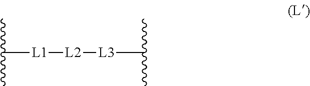

wherein:
LINK is attached to M via L1;
LINK is attached to U via L3;
L1 and L3 are each independently selected from a single bond, —N(R′)—, —C(O)N(R′)—, —O—, —N(R′)C(O)—, —C(O)—, —S(O₂)N(R′)—, —N(R′)S(O₂)—, —(C₁₋₆ alkylene)-, ethynyl, —(C₂₋₆ alkenylene)-, a divalent 3- to 6-membered cycloalkyl ring and a divalent 4- to 7-membered heterocyclyl ring;
L2 is represented by the formula -(L4)ₘ-;
each L4 is independently selected from a divalent 3- to 6-membered cycloalkyl ring, a divalent 4- to 7-membered heterocyclyl ring and a unit of formula:

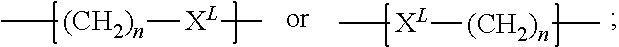

each X^{L} is independently selected from a single bond, —N(R′)—, —O—, —C(O)—, —S—, —SO—, —SO₂—, —C(H)═C(H)— and —C(H)—C(H)—;
n is selected from 1 to 4;
m is selected from 1 to 30; and
each R′ is independently selected from H and C₁₋₄ alkyl.

In one embodiment of formula (L′), L1 and L3 are each independently selected from a single bond, —N(R′)—, —C(O)N(R′)—, —O—, —N(R′)C(O)—, —C(O)—, —S(O₂)N(R′)—, —N(R′)S(O₂)—, —(C₁₋₆ alkylene)-, —(C₂₋₆ alkenylene)-, ethynyl, a divalent 3- to 6-membered cycloalkyl ring and a divalent 4- to 7-membered heterocyclyl ring;
L2 is represented by the formula -(L4)ₘ-; and
each L4 is independently selected from a divalent 3- to 6-membered cycloalkyl ring, a divalent 4- to 7-membered heterocyclyl ring and a unit of formula:

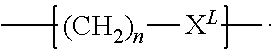

When m is not 1 (i.e. when there are two or more L4 groups present), then the X^{L} group of any L4 group is not directly bonded to the X^{L} group of any other L4 group that is present.

Typically, in formula (L′), L1 is selected from a single bond, branched or straight-chain —(C₁₋₆ alkylene)-, —C(O)N(R′)—, —N(R′)C(O)—, —O—, —S(O₂)N(R′)—, —N(R′)S(O₂)—, and a divalent 4- to 7-membered heterocyclyl ring. When present, the divalent 4- to 7-membered heterocyclyl ring of L1 typically contains at least one N atom, for instance one or two N atoms. Preferably, when L1 is a divalent 4- to 7-membered heterocyclyl ring, it is a divalent 5- to 6-membered heterocyclyl ring, more preferably a saturated divalent 5- to 6-membered heterocyclyl ring, for instance a divalent moiety of piperidine or diazinane. Preferably, in formula (L), L1 is selected from a single bond, branched or straight-chain —(C₁₋₆ alkylene)-, —C(O)N(H)—, —N(H)C(O)—, and a divalent moiety of piperidine or diazinane.

Typically, in formula (L′), L3 is selected from a single bond, —N(R′)—, —C(O)N(R′)—, —O—, —N(R′)C(O)—, —S(O₂)N(R′)—, —N(R′)S(O₂)—, ethynyl and a divalent 4- to 7-membered heterocyclyl ring. When present, the divalent 4- to 7-membered heterocyclyl ring of L3 typically contains at least one N atom, for instance one or two N atoms. Preferably, when L3 is a divalent 4- to 7-membered heterocyclyl ring, it is a divalent 5- to 6-membered heterocyclyl ring, more preferably a saturated divalent 5- to 6-membered heterocyclyl ring, for instance a divalent moiety of piperidine or diazinane, preferably 1,4-diazinane. Preferably, in formula (L), L3 is selected from a single bond, —N(H)—, —C(O)N(H)—, —O—, —N(H)C(O)—, ethynyl and a divalent moiety of piperidine or diazinane.

L2 is represented by the formula -(L4)ₘ-. Typically, in formula (L′), each L4 is independently selected from a divalent 4- to 7-membered heterocyclyl ring and a unit of formula

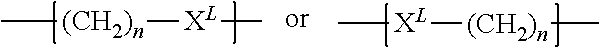

as described above. Typically, each X^{L} is independently selected from a single bond, —O—, —S—, —C(H)═C(H)— and —C(H)≡C(H)—, for example a single bond, —O— and —S—, and n is selected from 1 or 2. In one embodiment, each L4 is the same. Alternatively, L2 may comprise two or more blocks, wherein in each block, each L4 is the same. For instance, L2 may be represented by the formula -(L⁴′)ₚ-(L⁴″)_{q}-(L4″′)ᵣ-, wherein L4′, L4″ and L4″′ are selected from those moieties described above for L4, and wherein each L4′ is the same, each L4″ is the same and each L4″′ is the same. Preferably, L4′, L4″ and L4″′ are selected from —CH₂—, —CH₂CH₂O—, —OCH₂CH₂—, —CH₂CH₂S— and —SCH₂CH₂—. More preferably, L4′, L4″ and L4″′ are selected from —CH₂—, —CH₂CH₂O— and —OCH₂CH₂—. p, q and r are integers of from 0 to 30, wherein p+q+r=m.

In one embodiment of formula (L′), each L4 unit is the same. For instance, each L4 in formula (L′) may be —CH₂—, each L4 in formula (L′) may be —CH₂CH₂O—, each L4 in formula (L′) may be —OCH₂CH₂—, each L4 in formula (L′) may be —CH₂CH₂S—, or each L4 in formula (L′) may be —SCH₂CH₂—. More preferably, each L4 in formula (L′) is —CH₂—, each L4 in formula (L′) is —CH₂CH₂O— or each L4 in formula (L′) is —OCH₂CH₂—. Most preferably, each L4 in formula (L′) is —CH₂—.

Typically, in formula (L′), m is selected from 1 to 10.

Typically, in formula (L′), each R′ is independently selected from H and methyl. Preferably, each R′ is H.

When one or more L4 is a divalent 4- to 7-membered heterocyclyl ring, typically only one or two, e.g. one L4 is a divalent 4- to 7-membered heterocyclyl ring. Typically, therefore, none, one or two L4 is a divalent 4- to 7-membered heterocyclyl ring, for example none or one L4 is a divalent 4- to 7-membered heterocyclyl ring.

Typically, therefore, in formula (L′):
L1 is selected from a single bond, branched or straight-chain —(C₁₋₆ alkylene)-, —C(O)N(R′)—, —N(R′)C(O)—, —O—, —S(O₂)N(R′)—, —N(R′)S(O₂)—, and a divalent 4- to 7-membered heterocyclyl ring typically containing at least one N atom, for instance one or two N atoms;
L3 is selected from a single bond, —N(R′)—, —C(O)N(R′)—, —O—, —N(R′)C(O)—, —S(O₂)N(R′)—, —N(R′)S(O₂)—, ethynyl and a divalent 4- to 7-membered heterocyclyl ring typically containing at least one N atom, for instance one or two N atoms;
each L4 is independently selected from —CH₂—, —CH₂CH₂O—, —OCH₂CH₂—, —CH₂CH₂S—, —SCH₂CH₂—, and a divalent 4- to 7-membered heterocyclyl ring, wherein when one or more L4 is a divalent 4- to 7-membered heterocyclyl ring, only one or two, e.g. one L4 is a divalent 4- to 7-membered heterocyclyl ring;
m is selected from 1 to 10; and
each R′ is independently selected from H and methyl.

Preferably, in formula (L′):
L1 is selected from a single bond, branched or straight-chain —(C₁₋₆ alkylene)-, —C(O)N(H)—, —N(H)C(O)—, and a divalent moiety of piperidine or diazinane;
L3 is selected from a single bond, —N(H)—, —C(O)N(H)—, —O—, —N(H)C(O)—, ethynyl and a divalent moiety of piperidine or diazinane;
each L4 in formula (L) is —CH₂—, each L4 in formula (L) is —CH₂CH₂O—, each L4 in formula (L) is —OCH₂CH₂—, each L4 in formula (L) is —CH₂CH₂S—, or each L4 in formula (L) is —SCH₂CH₂—, with each L4 preferably being —CH₂—;
m is selected from 1 to 10; and
each R′ is H.

In a typical embodiment of formula (L′), L2 is —(C₁₋₁₀ alkylene)- or —(C₁₋₁₀ alkylene)-C(O)—, preferably —(C₁₋₁₀ alkylene)-. In this embodiment, L1 is preferably selected from a single bond, —C(O)N(H)—, —N(H)C(O)—, and a divalent moiety of piperidine or diazinane. In this embodiment, L3 is preferably selected from a single bond, —N(H)—, —C(O)N(H)—, —O—, —N(H)C(O)—, ethynyl and a divalent moiety of piperidine or diazinane. More preferably, L1 and L3 are selected from the following options:
L1 is a single bond and L3 is —N(H)—;
L1 is a divalent moiety of piperidine and L3 is —N(H)—;
L1 is —C(O)N(H)— or —N(H)C(O)—, and L3 is —C(O)N(H)— or —N(H)C(O)—;
L1 is a divalent moiety of piperidine and L3 is a divalent moiety of diazinane;
L1 is —C(O)N(H)— or —N(H)C(O)—, and L3 is a divalent moiety of diazinane;
L1 is a single bond and L3 is a divalent moiety of diazinane;
L1 is a single bond and L3 is a divalent moiety of piperidine;
L1 is a single bond and L3 is —O—;
L1 is a single bond and L3 is ethynyl;
L1 is a single bond and L3 is a single bond; and
L1 is —C(O)N(H)— or —N(H)C(O)—, and L3 is —O—.

For the avoidance of doubt, the skilled person would readily appreciate that each of the definitions of moieties U, M and LINK provided herein may be taken together in any combination to arrive at a definition of the compound of the present invention. As examples that are in no way limiting, definitions of moieties U, M and LINK labelled “typically” may be combined to arrive at a definition of a typical compound of the present invention, and definitions of moieties U, M and LINK labelled “preferably” may be combined to arrive at a definition of a preferred compound of the present invention. However, the skilled person would appreciate that a definition labelled “typically” of one moiety may be combined with a definition labelled “preferably” of another moiety to arrive at a definition of a compound of the present invention.

In preferred compounds of the present invention, M is of formula (III):

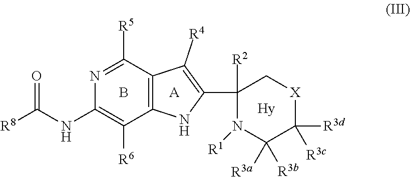

wherein R¹, R², R^{3a}, R^{3b}, R^{3c}, R^{3d}, X, R⁴, R, R⁶ and R⁸ are as defined herein.

Typically, in such preferred compounds:
R¹ is H or C₁₋₄ alkyl which is itself unsubstituted or substituted with C₁₋₄ alkoxy;
R² is H or methyl;
R^{3a} and R^{3b} are independently selected from H, C₁₋₄ alkyl, and C₁₋₄ alkoxy, or R^{3a} and R^{3b} form, together with the C atom to which they are attached, a C₃₋₆ cycloalkyl ring, R^{3c} and R^{3d} are independently selected from H, C₁₋₄ alkyl, halo and C₁₋₄ alkoxy, or R^{3c} and R^{3d} form, together with the C atom to which they are attached, a C₃₋₆ cycloalkyl ring, or
R^{3a} and R^{3c} are H, and R^{3b} and R^{3d} form, together with the C atoms to which they are attached, a C₅₋₆ cycloalkyl ring, with the proviso that when X is O or —N(Me)-, then neither R^{3c} nor R^{3d} are halo;
wherein at least two of R^{3a}, R^{3b}, R^{3c} and R^{3d} are H;
X is a bond, —N(Me)-, O or —CH₂—;
R⁴ is selected from H, halo C₁₋₄ alkoxy, and C₁₋₄ alkyl which is itself unsubstituted or substituted by one, two or three halo;
R⁵ and R⁶ are independently selected from H, halo C₁₋₄ alkoxy, and C₁₋₄ alkyl which is itself unsubstituted or substituted by one, two or three halo;
R⁸ is phenyl or a 5- to 6-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy; and
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo;
wherein either R⁸ is (i) a bond to LINK, or R⁸ is a group (ii) and M is bonded to LINK via a C or N atom within group R⁸ or ring Hy (as described herein) such that a hydrogen atom on the C or N atom within group R⁸ or ring Hy is replaced with a bond to LINK; and LINK is (a) a single bond, or (b) a chemical linker group represented by formula (L):

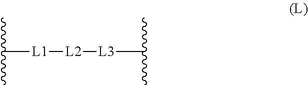

wherein:
LINK is attached to M via L1;
LINK is attached to U via L3;
L1 and L3 are each independently selected from a single bond, —N(R′)—, —C(O)N(R′)—, —O—, —N(R′)C(O)—, —C(O)—, —S(O₂)N(R′)—, —N(R′)S(O₂)—, —(C₁₋₆ alkylene)-, ethynylene, —(C₂₋₆ alkenylene)-, —C═C═C—, phenylene, a divalent 3- to 6-membered cycloalkyl ring and a divalent 4- to 7-membered heterocyclyl ring, the phenylene, cycloalkyl and heterocyclyl rings being unsubstituted or substituted by one or two C₁₋₄ alkyl;
L2 is represented by the formula -(L4)ₘ-;
each L4 is independently a unit of formula:

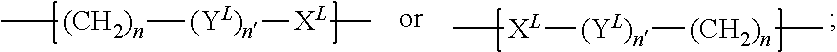

each X^{L} is independently selected from a single bond, —N(R′)—, —O—, —C(O)—, —S—, —SO—, —SO₂—, —C(R″)═C(R″)— and —C≡C—;
each Y^{L} is independently selected from a divalent 3- to 6-membered cycloalkyl ring, a divalent 4- to 7-membered heterocyclyl ring, or one of the following structures:

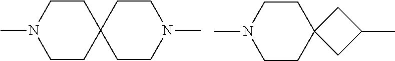

the cycloalkyl and heterocyclyl rings being unsubstituted or substituted by one or two C₁₋₄ alkyl; n is selected from 0 to 4;
n′ is selected from 0 or 1;
n+n′≥1;
m is selected from 1 to 10, preferably from 1 to 4 (for example 1, 2 or 3);
each R′ is independently selected from H and C₁₋₄ alkyl;
each R″ is independently selected from H and halo; U is:
(a) a CRBN E3 ubiquitin ligase binding moiety of formula (U1):

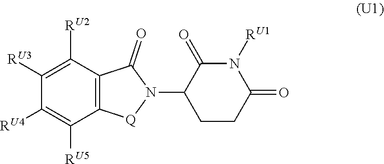

wherein:
R^{U1} is H;
Q is selected from —CH(R^{U6})—, —N(R^{U6})—, —O—, —C(O)—, —NH—CH(R^{U6})—, —N═C(R^{U6})—, or —N═N—;
R^{U6} is H or C₁₋₄alkyl;
R^{U2} and R^{U1} are each independently selected from H, halogen, C₁₋₄alkyl, NH₂, NO₂, OH, COOH, CN, CF₃, and a single bond to LINK;
R^{U3} and R^{U4} are each independently selected from H, halogen, C₁₋₄alkyl, NH₂, NO₂, OH, COOH, CN, CF₃, and a single bond to LINK;
wherein one and only one of R^{U2}, R^{U3}, R^{U4} and R^{U5} is a single bond to LINK;
(b) a CRBN E3 ubiquitin ligase binding moiety of formula (U4):

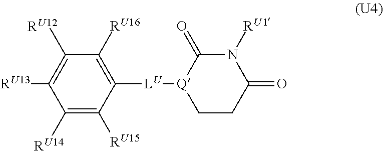

wherein:
R^{U1′} is H;
Q′ is N and L^{U} is a single bond, or Q′ is CH and L^{U} is selected from a single bond or —C(O)N(H)—, wherein either (i) the C atom of L^{U} is bonded to phenyl, and the N atom of L^{U} is bonded to Q′; or (ii) the C atom of L^{U} is bonded to Q′, and the N atom of L^{U} is bonded to phenyl;
R^{U12}, R^{U13} and R^{U14} are each independently selected from H, halogen, C₁₋₄ alkyl, NH₂, NO₂, OH, COOH, CN, CF₃, and a single bond to LINK;
wherein one and only one of R^{U12}, R^{U13} and R^{U14} is a single bond to LINK;
R^{U15} and R^{U16} are each independently selected from H, halogen, C₁₋₄ alkyl, NH₂, NO₂, OH, COOH, CN and CF₃;
(c) a CRBN E3 ubiquitin ligase binding moiety of formula (U5):

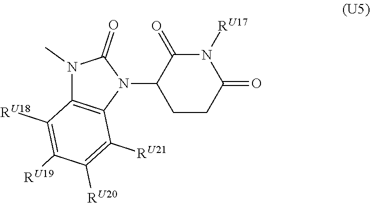

wherein:
R^{U17} is H;
R^{U18}, R^{U19}, R^{U20} and R^{U21} are each independently selected from H, halogen, C₁₋₄ alkyl, NH₂, NO₂, OH, COOH, CN, CF₃, and a single bond to LINK;
wherein one and only one of R^{U18}, R^{U19}, R^{U20} and R^{U21} is a single bond to LINK;
or (d) a VHL E3 ubiquitin ligase binding moiety of formula (U2):

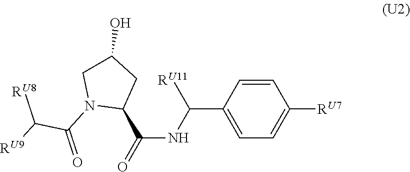

wherein:
R^{U7} is a group selected from phenyl, a 5- to 6-membered heteroaryl ring, a 5- to 6-membered heterocyclyl ring, and a 5- to 6-membered cycloalkyl ring, the group R^{U7} being unsubstituted or substituted by C₁₋₄ alkyl;
R^{U11} is H or C₁₋₄ alkyl;
R^{U8} is selected from C₁₋₄ alkyl, phenyl and a 3- to 8-membered cycloalkyl ring; and
R^{U9} is a single bond to LINK.

Alternatively, in such preferred compounds:
R¹ is H or C₁₋₄ alkyl which is itself unsubstituted or substituted with C₁₋₄ alkoxy;
R² is H or methyl;
R^{3a} and R^{3b} are independently selected from H, C₁₋₄ alkyl, and C₁₋₄ alkoxy, or R^{3a} and R^{3b} form, together with the C atom to which they are attached, a C₃₋₆ cycloalkyl ring, R^{3c} and R^{3d} are independently selected from H, C₁₋₄ alkyl, halo and C₁₋₄ alkoxy, or R^{3c} and R^{3d} form, together with the C atom to which they are attached, a C₃₋₆ cycloalkyl ring, or
R^{3a} and R^{3c} are H, and R^{3b} and R^{3d} form, together with the C atoms to which they are attached, a C₅₋₆ cycloalkyl ring, with the proviso that when X is 0 or —N(Me)-, then neither R^{3c} nor R^{3d} are halo;
wherein at least two of R^{3a}, R^{3b}, R^{3c} and R^{3d} are H;
X is a bond, —N(Me)-, O or —CH₂—;
R⁴ is selected from H, halo C₁₋₄ alkoxy, and C₁₋₄ alkyl which is itself unsubstituted or substituted by one, two or three halo;
R⁵ and R⁶ are independently selected from H, halo C₁₋₄ alkoxy, and C₁₋₄ alkyl which is itself unsubstituted or substituted by one, two or three halo;
R⁸ is phenyl or a 5- to 6-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy; and
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo;
wherein either R⁸ is (i) a bond to LINK, or R⁸ is a group (ii) and M is bonded to LINK via a C or N atom within group R⁸ or ring Hy (as described herein) such that a hydrogen atom on the C or N atom within group R⁸ or ring Hy is replaced with a bond to LINK; and
LINK is (a) a single bond, or (b) a chemical linker group represented by formula (L′):

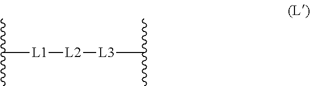

wherein:
LINK is attached to M via L1;
LINK is attached to U via L3;
L1 and L3 are each independently selected from a single bond, branched or straight-chain —(C₁₋₆ alkylene)-, —N(R′)—, —C(O)N(R′)—, —O—, —N(R′)C(O)—, —C(O)—, —S(O₂)N(R′)—, —N(R′)S(O₂)—, —(C₁₋₆ alkylene)-, ethynyl, —(C₂₋₆ alkenylene)-, a divalent 3- to 6-membered cycloalkyl ring and a divalent 4- to 7-membered heterocyclyl ring;
L2 is represented by the formula -(L4)ₘ-;
each L4 is independently selected from a divalent 3- to 6-membered cycloalkyl ring, a divalent
4- to 7-membered heterocyclyl ring and a unit of formula:

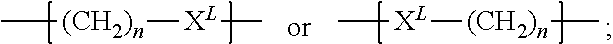

each X^{L} is independently selected from a single bond, —N(R′)—, —O—, —C(O)—, —S—, —SO—, —SO₂—, —C(H)═C(H)— and —C(H)—C(H)—;
n is selected from 1 to 4;
m is selected from 1 to 30;
each R′ is independently selected from H and C₁₋₄ alkyl; and
U is:
(a) a CRBN E3 ubiquitin ligase binding moiety of formula (U1):

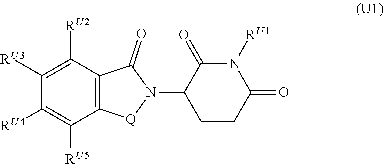

wherein:
R^{U1} is H;
Q is selected from —CH(R^{U6})—, —N(R^{U6})—, —O—, —C(O)—, —NH—CH(R^{U6})—, —N═C(R^{U6})—, or —N═N—;
R^{U6} is H or C₁₋₄alkyl;
R^{U2} and R^{U5} are each independently selected from H, halogen, C₁₋₄alkyl, NH₂, NO₂, OH, COOH, CN, CF₃, and a single bond to LINK;
R^{U3} and R^{U4} are each independently selected from H, halogen, C₁₋₄alkyl, NH₂, NO₂, OH, COOH, CN, CF₃, and a single bond to LINK;
wherein one and only one of R^{U2}, R^{U3}, R^{U4} and R^{U5} is a single bond to LINK;
(b) a CRBN E3 ubiquitin ligase binding moiety of formula (U4):

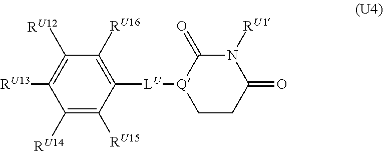

wherein:
R^{U1} is H;
Q′ is N and L^{U} is a single bond, or Q′ is CH and L^{U} is selected from a single bond or —C(O)N(H)—, wherein either (i) the C atom of L^{U} is bonded to phenyl, and the N atom of L^{U} is bonded to Q′; or (ii) the C atom of L^{U} is bonded to Q′, and the N atom of L^{U} is bonded to phenyl;
R^{U12}, R^{U13} and R^{U14} are each independently selected from H, halogen, C₁₋₄ alkyl, NH₂, NO₂, OH, COOH, CN, CF₃, and a single bond to LINK;
wherein one and only one of R^{U12}, R^{U13} and R^{U14} is a single bond to LINK;
R^{U15} and R^{U16} are each independently selected from H, halogen, C₁₋₄ alkyl, NH₂, NO₂, OH, COOH, CN and CF₃;
(c) a CRBN E3 ubiquitin ligase binding moiety of formula (U5):

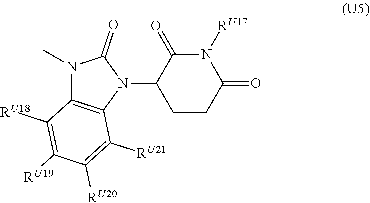

wherein:
R^{U17} is H;
R^{U18}, R^{U19}, R^{U20} and R^{U21} are each independently selected from H, halogen, C₁₋₄ alkyl, NH₂, NO₂, OH, COOH, CN, CF₃, and a single bond to LINK;
wherein one and only one of R^{U18}, R^{U19}, R^{U20} and R^{U21} is a single bond to LINK;
or (d) a VHL E3 ubiquitin ligase binding moiety of formula (U2):

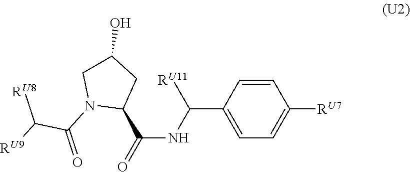

wherein:
R^{U7} is a group selected from phenyl, a 5- to 6-membered heteroaryl ring, a 5- to 6-membered heterocyclyl ring, and a 5- to 6-membered cycloalkyl ring, the group R^{U7} being unsubstituted or substituted by C₁₋₄ alkyl;
R^{U11} is H or C₁₋₄ alkyl;
R^{U8} is selected from C₁₋₄ alkyl, phenyl and a 3- to 8-membered cycloalkyl ring; and
R^{U9} is a single bond to LINK.

In one preferred aspect of these embodiments, M is of formula (IV):

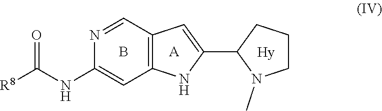

wherein R⁸ is as described herein, the group R⁸ being unsubstituted or substituted by one, two or three substituents independently selected from R¹⁰, halo and C₁₋₄ alkyl;
wherein either R⁸ is (i) a bond to LINK, or R⁸ is a group (ii) and M is bonded to LINK via a C or N atom within group R⁸ such that a hydrogen atom on the C or N atom within group R⁸ is replaced with a bond to LINK.

In this embodiment, R⁸ is preferably selected from one of the following structures:

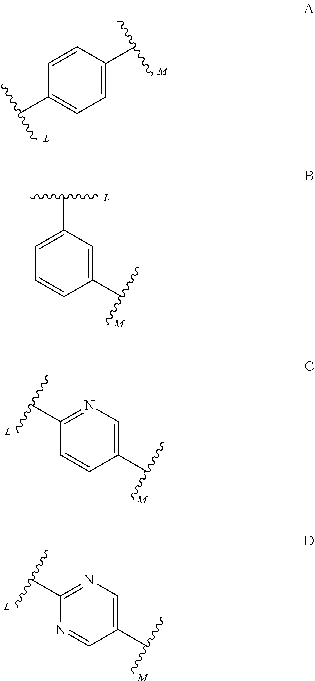

wherein:

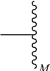

represents the point of attachment of R⁸ to the rest of moiety M;

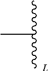

represents the point of attachment of R⁸ to LINK or R¹⁰; and
the group R⁸ is further unsubstituted or substituted by one or two substituents independently selected from R¹⁰, fluorine and methyl. For example, the group R⁸ may be selected from one of the following structures:

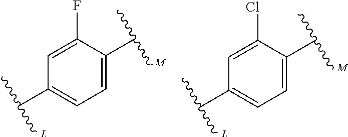

wherein:

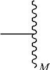

represents the point of attachment of R⁸ to the rest of moiety M; and

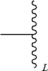

represents the point of attachment of R⁸ to LINK.

Alternatively, the group R⁸ may be unsubstituted phenylene, for instance as depicted by structure A above.

In this embodiment, L2 is preferably selected from —(CH₂)ₘ—, —(CH₂CH₂O)ₘ—, —(OCH₂CH₂)ₘ—, —(CH₂CH₂S)ₘ—, —(SCH₂CH₂)ₘ—, and a divalent 4- to 7-membered heterocyclyl ring, preferably from —(CH₂)ₘ and a divalent 4- to 7-membered heterocyclyl ring.

In this embodiment, preferably L1 and L3 are each independently selected from a single bond, branched or straight-chain —(C₁₋₆ alkylene)-, —N(R′)—, —C(O)N(R′)—, —N(R′)C(O)—, —O—, ethynyl and a divalent 4- to 7-membered heterocyclyl ring, preferably L1 and L3 are each independently selected from a single bond, branched or straight-chain —(C₁₋₆ alkylene)-, —N(R′)—, —C(O)N(R′)—, —N(R′)C(O)—, ethynyl, piperidinyl and diazinanyl. Preferably in this embodiment, R¹ is H and the divalent 4- to 7-membered heterocyclyl ring is piperidine or diazinane. More preferably, L1 is a single bond, L2 is —(C₁₋₁₀ alkylene)- and L3 is ethynylene or a divalent moiety of piperidine (for instance ethynylene), preferably wherein L2 is —(C₃₋₈ alkylene)-, more preferably wherein L2 is —(C₇ alkylene)- or —(C₃ alkylene)-.

In this embodiment, U is preferably (i) of formula (U1) wherein preferably Q is selected from —CH₂— and —C(O)—, R^{U6} is H, one of R^{U2}, R^{U3}, R^{U4} and R^{U5} is a single bond to LINK, and the rest are H, for instance wherein R^{U2} and R^{U3} are H, one of R^{U4} and R^{U5} is a single bond to LINK, and the other of R^{U4} and R^{U5} is H, or (ii) of formula (U3) wherein preferably V is S, W is N, R^{U10} is methyl, R^{U11} is H, and R^{U5} is t-butyl or phenyl, more preferably t-butyl. Most preferably, in formula (U1): Q is —CH₂—; R^{U6} is H; and R^{U2}, R^{U3} and R^{U4} are H, and R^{U5} is a single bond to LINK.

Particularly preferred compounds of the invention may be selected from:
4-(4-(2-(2,6-dioxopiperidin-3-yl)-1-oxoisoindolin-4-yl)but-3-yn-1-yl)-N-(2-((R)-1-methylpyrrolidin-2-yl)-1H-pyrrolo[3,2-c]pyridin-6-yl)piperidine-1-carboxamide;
6-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)nicotinamide;
4-((1-(2-(2,6-dioxopiperidin-3-yl)-1,3-dioxoisoindolin-4-yl)piperidin-4-yl)methyl)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1,3-dioxoisoindolin-4-yl)piperidin-4-yl)ethyl)-N-(2-((R)-1-methylpyrrolidin-2-yl)-1H-pyrrolo[3,2-c]pyridin-6-yl)benzamide;
6-(4-((1-(2-(2,6-dioxopiperidin-3-yl)-1,3-dioxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)nicotinamide;
6-(4-(3-(2-(2,6-dioxopiperidin-3-yl)-1-oxoisoindolin-4-yl)prop-2-yn-1-yl)piperazin-1-yl)-N-(2-((R)-1-methylpyrrolidin-2-yl)-1H-pyrrolo[3,2-c]pyridin-6-yl)nicotinamide;
N—((S)-2,6-dioxopiperidin-3-yl)-6-(6-(4-((2-((R)-1-methylpyrrolidin-2-yl)-1H-pyrrolo[3,2-c]pyridin-6-yl)carbamoyl)phenyl)hex-1-yn-1-yl)picolinamide;
4-(6-(2-(2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide;
(R)-4-(6-(6-(2,4-dioxotetrahydropyrimidin-1(2H)-yl)pyridin-2-yl)hex-5-yn-1-yl)-N-(2-(1-methylpyrrolidin-2-yl)-1H-pyrrolo[3,2-c]pyridin-6-yl)benzamide;
6-(6-(2-(2,6-dioxopiperidin-3-yl)-1-oxoisoindolin-4-yl) hex-5-yn-1-yl)-N-(2-((R)-1-methylpyrrolidin-2-yl)-1H-pyrrolo[3,2-c]pyridin-6-yl) nicotinamide;
N—((S)-2,6-dioxopiperidin-3-yl)-5-(5-(4-((2-((R)-1-methylpyrrolidin-2-yl)-1H-pyrrolo[3,2-c]pyridin-6-yl)carbamoyl)phenyl)pent-1-yn-1-yl)picolinamide;
6-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)nicotinamide;
N-(3-chloro-2-((R)-1-methylpyrrolidin-2-yl)-1H-pyrrolo[3,2-c]pyridin-6-yl)-9-(2-(2,6-dioxopiperidin-3-yl)-1-oxoisoindolin-4-yl) non-8-ynamide;
3-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)benzamide;
4-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)benzamide;
6-(4-(3-(2-(2,6-dioxopiperidin-3-yl)-1-oxoisoindolin-4-yl)prop-2-yn-1-yl)piperidin-1-yl)-N-(2-((R)-1-methylpyrrolidin-2-yl)-1H-pyrrolo[3,2-c]pyridin-6-yl)nicotinamide;
6-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)nicotinamide;
6-(4-((1-(2-(2,6-dioxopiperidin-3-yl)-1,3-dioxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)nicotinamide;
4-((4-(2-(2,6-dioxopiperidin-3-yl)-1-oxoisoindolin-4-yl)but-3-yn-1-yl)oxy)-N-(2-((R)-1-methylpyrrolidin-2-yl)-1H-pyrrolo[3,2-c]pyridin-6-yl)benzamide;
4-(1-(5-(2-(2,6-dioxopiperidin-3-yl)-1-oxoisoindolin-4-yl)pent-4-yn-1-yl)-1H-pyrazol-3-yl)-N-(2-((R)-1-methylpyrrolidin-2-yl)-1H-pyrrolo[3,2-c]pyridin-6-yl)benzamide;
4-(1-(5-(2-(2,6-dioxopiperidin-3-yl)-1-oxoisoindolin-4-yl)pent-4-yn-1-yl)-1H-pyrazol-4-yl)-N-(2-((R)-1-methylpyrrolidin-2-yl)-1H-pyrrolo[3,2-c]pyridin-6-yl)benzamide;
4-(1-(5-(2-(2,6-dioxopiperidin-3-yl)-1-oxoisoindolin-5-yl)pent-4-yn-1-yl)-1H-pyrazol-4-yl)-N-(2-((R)-1-methylpyrrolidin-2-yl)-1H-pyrrolo[3,2-c]pyridin-6-yl)benzamide;
3-chloro-4-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)benzamide;
4-(((2S)-4-(2-(2,6-dioxopiperidin-3-yl)-1-oxoisoindolin-4-yl)but-3-yn-2-yl)oxy)-N-(2-((R)-1-methylpyrrolidin-2-yl)-1H-pyrrolo[3,2-c]pyridin-6-yl)benzamide;
4-(((2R)-4-(2-(2,6-dioxopiperidin-3-yl)-1-oxoisoindolin-4-yl)but-3-yn-2-yl)oxy)-N-(2-((R)-1-methylpyrrolidin-2-yl)-1H-pyrrolo[3,2-c]pyridin-6-yl)benzamide;
3-((9-(2-(2,6-dioxopiperidin-3-yl)-1,3-dioxoisoindolin-4-yl)non-8-yn-1-yl)oxy)-N-(2-((R)-1-methylpyrrolidin-2-yl)-1H-pyrrolo[3,2-c]pyridin-6-yl)benzamide;
7-{[2-(2,6-dioxopiperidin-3-yl)-1,3-dioxoisoindol-5-yl]amino}-N-{2-[(2R)-1-methylpyrrolidin-2-yl]-1H-pyrrolo[3,2-c]pyridin-6-yl}heptanamide;
7-{[2-(2,6-dioxopiperidin-3-yl)-1,3-dioxoisoindol-5-yl]amino}-N-{2-[(2S)-1-methylpyrrolidin-2-yl]-1H-pyrrolo[3,2-c]pyridin-6-yl}heptanamide;
7-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}heptanamide;
9-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}nonanamide;
12-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}dodecanamide;
12-{[2-(2,6-dioxopiperidin-3-yl)-1,3-dioxoisoindol-4-yl]amino}-N-{2-[(2S)-1-methylpyrrolidin-2-yl]-1H-pyrrolo[3,2-c]pyridin-6-yl}dodecanamide;
N-{3-chloro-2-[(2R)-1-methylpyrrolidin-2-yl]-1H-pyrrolo[3,2-c]pyridin-6-yl}-7-{[2-(2,6-dioxopiperidin-3-yl)-1,3-dioxoisoindol-4-yl]amino}heptanamide;
4-[1-(3-{1-[2-(2,6-dioxopiperidin-3-yl)-1,3-dioxoisoindol-4-yl]piperidin-4-yl}propyl)pyrazol-4-yl]-N-{2-[(2R)-1-methylpyrrolidin-2-yl]-1H-pyrrolo[3,2-c]pyridin-6-yl}benzamide;
4-[1-(3-{1-[2-(2,6-dioxopiperidin-3-yl)-1-oxo-3H-isoindol-4-yl]piperidin-4-yl}propyl)pyrazol-4-yl]-N-{2-[(2R)-1-methylpyrrolidin-2-yl]-1H-pyrrolo[3,2-c]pyridin-6-yl}benzamide;
4-(3-{[2-(2,6-dioxopiperidin-3-yl)-1,3-dioxoisoindol-4-yl]amino}propyl)-N-{2-[(2R)-1-methylpyrrolidin-2-yl]-1H-pyrrolo[3,2-c]pyridin-6-yl}benzamide;
4-(3-{[2-(2,6-dioxopiperidin-3-yl)-1,3-dioxoisoindol-4-yl]amino}propyl)-N-{2-[(2S)-1-methylpyrrolidin-2-yl]-1H-pyrrolo[3,2-c]pyridin-6-yl}benzamide;
1-(3-{1-[2-(2,6-dioxopiperidin-3-yl)-1,3-dioxoisoindol-4-yl]piperidin-4-yl}propyl)-N-{2-[(2R)-1-methylpyrrolidin-2-yl]-1H-pyrrolo[3,2-c]pyridin-6-yl}pyrazole-4-carboxamide;
2-(((1r,4R)-4-((2-(2,6-dioxopiperidin-3-yl)-1,3-dioxoisoindolin-4-yl)amino)cyclohexyl)oxy)-N-(2-((R)-1-methylpyrrolidin-2-yl)-1H-pyrrolo[3,2-c]pyridin-6-yl)acetamide;
2-(4-((2-(2,6-dioxopiperidin-3-yl)-1,3-dioxoisoindolin-4-yl)amino)butoxy)-N-(2-((R)-1-methylpyrrolidin-2-yl)-1H-pyrrolo[3,2-c]pyridin-6-yl)acetamide;
4-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)butanamide;
5-(1-(2-(2,6-Dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)pentanamide;
6-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)hexanamide;
4-(1-(2-(2,6-dioxopiperidin-3-yl)-1,3-dioxoisoindolin-4-yl)piperidin-3-yl)-N-(2-((R)-1-methylpyrrolidin-2-yl)-1H-pyrrolo[3,2-c]pyridin-6-yl)butanamide;
3-(3-((2-(2,6-dioxopiperidin-3-yl)-1,3-dioxoisoindolin-4-yl)amino)propyl)-N-(2-((R)-1-methylpyrrolidin-2-yl)-1H-pyrrolo[3,2-c]pyridin-6-yl)benzamide;
3-(((1-(2-(2,6-dioxopiperidin-3-yl)-1,3-dioxoisoindolin-4-yl)piperidin-4-yl)methyl)amino)-N-(2-((R)-1-methylpyrrolidin-2-yl)-1H-pyrrolo[3,2-c]pyridin-6-yl)benzamide;
3-(1-(3-(1-(2-(2,6-dioxopiperidin-3-yl)-1,3-dioxoisoindolin-4-yl)piperidin-4-yl)propyl)-1H-pyrazol-4-yl)-N-(2-((R)-1-methylpyrrolidin-2-yl)-1H-pyrrolo[3,2-c]pyridin-6-yl)benzamide;
4-(1-(2-(2,6-dioxopiperidin-3-yl)-1,3-dioxoisoindolin-4-yl)azetidin-3-yl)-N-(2-((R)-1-methylpyrrolidin-2-yl)-1H-pyrrolo[3,2-c]pyridin-6-yl)butanamide;
5-(4-(2-(2,6-dioxopiperidin-3-yl)-1-oxoisoindolin-4-yl)but-3-yn-1-yl)-N-(2-((R)-1-methylpyrrolidin-2-yl)-1H-pyrrolo[3,2-c]pyridin-6-yl)picolinamide;
5-[3-[2-(2,6-dioxo-3-piperidyl)-1-oxo-isoindolin-4-yl]prop-2-ynoxy]-N-[2-(1-methylpyrrolidin-2-yl)-1H-pyrrolo[3,2-c]pyridin-6-yl]pyridine-2-carboxamide;
4-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)benzamide;
4-(4-(2-(2,6-dioxopiperidin-3-yl)-1-oxoisoindolin-4-yl)but-3-yn-1-yl)-N-(2-((R)-1-methylpyrrolidin-2-yl)-1H-pyrrolo[3,2-c]pyridin-6-yl)benzamide;
4-(4-(1-(2,6-dioxopiperidin-3-yl)-3-methyl-2-oxo-2,3-dihydro-1H-benzo[d]imidazol-4-yl)but-3-yn-1-yl)-N-(2-((R)-1-methylpyrrolidin-2-yl)-1H-pyrrolo[3,2-c]pyridin-6-yl)benzamide;
4-(4-(2-(2,6-dioxopiperidin-3-yl)-1-oxoisoindolin-4-yl) but-3-yn-1-yl)-2-fluoro-N-(2-((R)-1-methylpyrrolidin-2-yl)-1H-pyrrolo[3,2-c]pyridin-6-yl)benzamide;
4-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
4-((5-(2-(2,6-dioxopiperidin-3-yl)-1-oxoisoindolin-4-yl)pent-4-yn-1-yl)oxy)-N-(2-((R)-1-methylpyrrolidin-2-yl)-1H-pyrrolo[3,2-c]pyridin-6-yl)benzamide;
4-((3-(2-(2,6-dioxopiperidin-3-yl)-1-oxoisoindolin-4-yl)prop-2-yn-1-yl)oxy)-2-fluoro-N-(2-((R)-1-methylpyrrolidin-2-yl)-1H-pyrrolo[3,2-c]pyridin-6-yl)benzamide;
3-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)benzamide;
4-(1-(6-(2-(2,6-dioxopiperidin-3-yl)-1-oxoisoindolin-4-yl)hex-5-yn-1-yl)-1H-pyrazol-4-yl)-N-(2-((R)-1-methylpyrrolidin-2-yl)-1H-pyrrolo[3,2-c]pyridin-6-yl)benzamide;
N—((S)-2,6-dioxopiperidin-3-yl)-6-(4-(3-(4-(4-((2-((R)-1-methylpyrrolidin-2-yl)-1H-pyrrolo[3,2-c]pyridin-6-yl)carbamoyl)phenyl)-1H-pyrazol-1-yl)propyl)piperidin-1-yl)picolinamide;
N—((S)-2,6-dioxopiperidin-3-yl)-6-(4-(2-(4-(4-((2-((R)-1-methylpyrrolidin-2-yl)-1H-pyrrolo[3,2-c]pyridin-6-yl)carbamoyl)phenyl)-1H-pyrazol-1-yl)ethoxy)piperidin-1-yl)picolinamide;
N—((S)-2,6-dioxopiperidin-3-yl)-6-(5-(4-(4-((2-((R)-1-methylpyrrolidin-2-yl)-1H-pyrrolo[3,2-c]pyridin-6-yl)carbamoyl)phenyl)-1H-pyrazol-1-yl)pent-1-yn-1-yl) picolinamide;
(R)-4-(5-(6-(2,4-dioxotetrahydropyrimidin-1(2H)-yl)pyridin-2-yl)pent-4-yn-1-yl)-N-(2-(1-methylpyrrolidin-2-yl)-1H-pyrrolo[3,2-c]pyridin-6-yl)benzamide;
6-(4-(4-(2-(2,6-dioxopiperidin-3-yl)-1,3-dioxoisoindolin-4-yl)piperazin-1-yl)piperidin-1-yl)-N-(2-((R)-1-methylpyrrolidin-2-yl)-1H-pyrrolo[3,2-c]pyridin-6-yl)nicotinamide;
2-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)pyrimidine-5-carboxamide;
4-((3-(1-(2,6-dioxopiperidin-3-yl)-3-methyl-2-oxo-2,3-dihydro-1H-benzo[d]imidazol-4-yl)prop-2-yn-1-yl)oxy)-N-(2-((R)-1-methylpyrrolidin-2-yl)-1H-pyrrolo[3,2-c]pyridin-6-yl)benzamide;
6-(4-(4-(2-(2,6-dioxopiperidin-3-yl)-1-oxoisoindolin-4-yl)phenyl)piperidin-1-yl)-N-(2-((R)-1-methylpyrrolidin-2-yl)-1H-pyrrolo[3,2-c]pyridin-6-yl)nicotinamide;
7-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}heptanamide;
(2S,4R)-1-[(2S)-3,3-dimethyl-2-(11-{[4-({2-[(2R)-1-methylpyrrolidin-2-yl]-1H-pyrrolo[3,2-c]pyridin-6-yl}carbamoyl)phenyl]formamido}undecanamido)butanoyl]-4-hydroxy-N-{[4-(4-methyl-1,3-thiazol-5-yl)phenyl]methyl}pyrrolidine-2-carboxamide;
(2S,4R)-1-[(2S)-3,3-dimethyl-2-(11-{[4-({2-[(2S)-1-methylpyrrolidin-2-yl]-1H-pyrrolo[3,2-c]pyridin-6-yl}carbamoyl)phenyl]formamido}undecanamido)butanoyl]-4-hydroxy-N-{[4-(4-methyl-1,3-thiazol-5-yl)phenyl]methyl}pyrrolidine-2-carboxamide;
N-(3-chloro-2-((R)-1-methylpyrrolidin-2-yl)-1H-pyrrolo[3,2-c]pyridin-6-yl)-6-(6-(2-(2,6-dioxopiperidin-3-yl)-1-oxoisoindolin-4-yl)hex-5-yn-1-yl)nicotinamide;
N-(3-chloro-2-((R)-1-methylpyrrolidin-2-yl)-1H-pyrrolo[3,2-c]pyridin-6-yl)-6-(4-(3-(2-(2,6-dioxopiperidin-3-yl)-1-oxoisoindolin-4-yl)prop-2-yn-1-yl)piperazin-1-yl)nicotinamide;
6-(4-(3-(2-(2,6-dioxopiperidin-3-yl)-1-oxoisoindolin-4-yl)prop-2-yn-1-yl)piperidin-1-yl)-N-(2-((R)-1-ethylpyrrolidin-2-yl)-1H-pyrrolo[3,2-c]pyridin-6-yl)nicotinamide;
6-(6-(2-(2,6-dioxopiperidin-3-yl)-1-oxoisoindolin-4-yl)hex-5-yn-1-yl)-N-(2-((R)-1-ethylpyrrolidin-2-yl)-1H-pyrrolo[3,2-c]pyridin-6-yl)nicotinamide;
6-(1′-(2-(2,6-dioxopiperidin-3-yl)-1-oxoisoindolin-4-yl)-[4,4′-bipiperidin]-1-yl)-N-(2-((R)-1-methylpyrrolidin-2-yl)-1H-pyrrolo[3,2-c]pyridin-6-yl)nicotinamide;
4-(3-(1-(2-(2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide;
4-(3-(1-(2-((rel-S)-2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide;
4-(3-(1-(2-((rel-R)-2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide;
4-(2-(1-(2-((rel-S)-2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide;
4-(2-(1-(2-((rel-R)-2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1,3-dioxoisoindolin-4-yl) piperidin-4-yl) ethoxy)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1,3-dioxoisoindolin-5-yl)piperidin-4-yl)ethoxy)-N-(2-((R)-1-methylpyrrolidin-2-yl)-1H-pyrrolo[3,2-c]pyridin-6-yl)benzamide;
4-(5-(2-(2,6-dioxopiperidin-3-yl)-1-oxoisoindolin-4-yl)pent-4-yn-1-yl)-3,5-difluoro-N-(2-((R)-1-methylpyrrolidin-2-yl)-1H-pyrrolo[3,2-c]pyridin-6-yl benzamide;
4-(4-((4-(2-(2,6-dioxopiperidin-3-yl)-1-oxoisoindolin-4-yl)benzyl)oxy)piperidin-1-yl)-N-(2-((R)-1-methylpyrrolidin-2-yl)-1H-pyrrolo[3,2-c]pyridin-6-yl)benzamide;
6-(6-(2-(2,6-dioxopiperidin-3-yl)-1-oxoisoindolin-4-yl)hex-5-yn-1-yl)-N-(2-((rel-R)-5-methyl-5-azaspiro[2.4]heptan-6-yl)-1H-pyrrolo[3,2-c]pyridin-6-yl)nicotinamide;
4-((4-(2-(2,6-dioxopiperidin-3-yl)-1-oxoisoindolin-4-yl)but-3-yn-1-yl) (methyl)amino)-N-(2-((R)-1-methylpyrrolidin-2-yl)-1H-pyrrolo[3,2-c]pyridin-6-yl)benzamide;
4-(4-(2-(1-(2-(2,6-dioxopiperidin-3-yl)-1-oxoisoindolin-4-yl)piperidin-4-yl)acetyl) piperazin-1-yl)-N-(2-((R)-1-methylpyrrolidin-2-yl)-1H-pyrrolo[3,2-c]pyridin-6-yl)benzamide;
2-(4-(3-(2-(2,6-dioxopiperidin-3-yl)-1-oxoisoindolin-4-yl)prop-2-yn-1-yl)piperazin-1-yl)-N-(2-((R)-1-methylpyrrolidin-2-yl)-1H-pyrrolo[3,2-c]pyridin-6-yl)pyrimidine-5-carboxamide;
4-(5-(2-(2,6-dioxopiperidin-3-yl)-7-fluoro-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
4-(4-((4-(2-(2,6-dioxopiperidin-3-yl)-1-oxoisoindolin-4-yl)piperazin-1-yl)methyl)piperidin-1-yl)-N-(2-((R)-1-methylpyrrolidin-2-yl)-1H-pyrrolo[3,2-c]pyridin-6-yl)benzamide;
4-(5-(2-(2,6-dioxopiperidin-3-yl)-1-oxoisoindolin-4-yl)pent-4-yn-1-yl)-3-fluoro-N-(2-((R)-1-methylpyrrolidin-2-yl)-1H-pyrrolo[3,2-c]pyridin-6-yl)benzamide;
4-(5-(2-(2,6-dioxopiperidin-3-yl)-1-oxoisoindolin-4-yl)pent-4-yn-1-yl)-2-fluoro-N-(2-((R)-1-methylpyrrolidin-2-yl)-1H-pyrrolo[3,2-c]pyridin-6-yl)benzamide;
3-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
3-(6-(2-(2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide;
N—((S)-2,6-dioxopiperidin-3-yl)-6-(5-(4-((2-((R)-1-methylpyrrolidin-2-yl)-1H-pyrrolo[3,2-c]pyridin-6-yl)carbamoyl)phenyl)pent-1-yn-1-yl)picolinamide;
4-(4-(4-(2-(2,6-dioxopiperidin-3-yl)-1-oxoisoindolin-4-yl)benzyl)piperazin-1-yl)-N-(2-((R)-1-methylpyrrolidin-2-yl)-1H-pyrrolo[3,2-c]pyridin-6-yl)benzamide;
6-(4-(4-(2-(2,6-dioxopiperidin-3-yl)-1-oxoisoindolin-4-yl)-3-methylphenyl)piperidin-1-yl)-N-(2-((R)-1-methylpyrrolidin-2-yl)-1H-pyrrolo[3,2-c]pyridin-6-yl)nicotinamide;
N-(3-chloro-2-((R)-1-methylpyrrolidin-2-yl)-1H-pyrrolo[3,2-c]pyridin-6-yl)-4-(5-(2-(2,6-dioxopiperidin-3-yl)-1-oxoisoindolin-4-yl)pent-4-yn-1-yl)benzamide;
4-(5-(2-(2,6-dioxopiperidin-3-yl)-6-fluoro-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
4-(5-(2-((rel-S)-2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
4-(5-(2-((rel-R)-2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
4-(6-(2-((rel-S)-2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide;
4-(6-(2-((rel-R)-2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide;
4-(5-(2-(2,6-dioxopiperidin-3-yl)-6-fluoro-1-oxoisoindolin-5-yl)pent-4-yn-1-yl)-N-(2-((R)-1-methylpyrrolidin-2-yl)-1H-pyrrolo[3,2-c]pyridin-6-yl)benzamide;
4-(1-(2-(1-(2-(2,6-dioxopiperidin-3-yl)-1-oxoisoindolin-4-yl)piperidin-4-yl)acetyl)piperidin-4-yl)-N-(2-((R)-1-methylpyrrolidin-2-yl)-1H-pyrrolo[3,2-c]pyridin-6-yl)benzamide;
6-(4-(2-(1-(2-(2,6-dioxopiperidin-3-yl)-1-oxoisoindolin-4-yl)piperidin-4-yl)acetyl)piperazin-1-yl)-N-(2-((R)-1-methylpyrrolidin-2-yl)-1H-pyrrolo[3,2-c]pyridin-6-yl)nicotinamide;
6-(4-((4-(2-(2,6-dioxopiperidin-3-yl)-1-oxoisoindolin-4-yl)benzyl)oxy)piperidin-1-yl)-N-(2-((R)-1-methylpyrrolidin-2-yl)-1H-pyrrolo[3,2-c]pyridin-6-yl)nicotinamide;
4-(5-(2-(2,6-dioxopiperidin-3-yl)-1,3-dioxoisoindolin-4-yl)pent-4-yn-1-yl)-N-(2-((R)-1-methylpyrrolidin-2-yl)-1H-pyrrolo[3,2-c]pyridin-6-yl)benzamide;
4-(5-(2-(2,6-dioxopiperidin-3-yl)-1-oxoisoindolin-4-yl)pent-4-yn-1-yl)-N-(2-((R)-1-(methyl-d3)pyrrolidin-2-yl)-1H-pyrrolo[3,2-c]pyridin-6-yl)benzamide;
4-(6-(2-(2,6-dioxopiperidin-3-yl)-1-oxoisoindolin-4-yl)hex-5-yn-1-yl)-2-fluoro-N-(2-((R)-1-methylpyrrolidin-2-yl)-1H-pyrrolo[3,2-c]pyridin-6-yl)benzamide;
N-(3-chloro-2-((R)-1-methylpyrrolidin-2-yl)-1H-pyrrolo[3,2-c]pyridin-6-yl)-4-(6-(2-(2,6-dioxopiperidin-3-yl)-1-oxoisoindolin-4-yl)hex-5-yn-1-yl)-2-fluorobenzamide;
4-(6-(2-(2,6-dioxopiperidin-3-yl)-6-fluoro-1-oxoisoindolin-5-yl)hex-5-yn-1-yl)-2-fluoro-N-(2-((R)-1-methylpyrrolidin-2-yl)-1H-pyrrolo[3,2-c]pyridin-6-yl)benzamide;
4-(6-(2-(2,6-dioxopiperidin-3-yl)-1-oxoisoindolin-4-yl)hex-5-yn-1-yl)-2-methoxy-N-(2-((R)-1-methylpyrrolidin-2-yl)-1H-pyrrolo[3,2-c]pyridin-6-yl)benzamide;
4-(4-((1-(2-(2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)benzamide;
4-(4-((1-(2-((rel-S)-2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)benzamide;
4-(4-((1-(2-((rel-R)-2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)benzamide;
6-(1-((1-(2-(2,6-dioxopiperidin-3-yl)-1-oxoisoindolin-4-yl)piperidin-4-yl)methyl)piperidin-4-yl)-N-(2-((R)-1-methylpyrrolidin-2-yl)-1H-pyrrolo[3,2-c]pyridin-6-yl)nicotinamide;
4-(4-((1-(2-(2,6-dioxopiperidin-3-yl)-1-oxoisoindolin-4-yl)piperidin-4-yl)methyl)piperazin-1-yl)-2-fluoro-6-methoxy-N-(2-((R)-1-methylpyrrolidin-2-yl)-1H-pyrrolo[3,2-c]pyridin-6-yl)benzamide;
4-((1-((1-(2-(2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
4-((1-((1-(2-((rel-S)-2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
4-((1-((1-(2-((rel-R)-2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
4-((1-((1-(2-(2,6-dioxopiperidin-3-yl)-1,3-dioxoisoindolin-5-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
6-((1-((1-(2-(2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)nicotinamide;
6-(4-((1-(2-(2,6-dioxopiperidin-3-yl)-1-oxoisoindolin-4-yl)piperidin-4-yl)methyl)piperazin-1-yl)-2-methoxy-N-(2-((R)-1-methylpyrrolidin-2-yl)-1H-pyrrolo[3,2-c]pyridin-6-yl)nicotinamide;
6-((2S)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)nicotinamide;
4-(3-(1-(2-(2,6-dioxopiperidin-3-yl)-1,3-dioxoisoindolin-4-yl)piperidin-4-yl)propyl)-N-(2-((R)-1-methylpyrrolidin-2-yl)-1H-pyrrolo[3,2-c]pyridin-6-yl)benzamide;
6-(4-(4-(2-(2,6-dioxopiperidin-3-yl)-1-oxoisoindolin-4-yl)benzyl)piperazin-1-yl)-N-(2-((R)-1-methylpyrrolidin-2-yl)-1H-pyrrolo[3,2-c]pyridin-6-yl)nicotinamide;
4-(3-(4-(2-(2,6-dioxopiperidin-3-yl)-1,3-dioxoisoindolin-4-yl)piperazin-1-yl)propyl)-N-(2-((R)-1-methylpyrrolidin-2-yl)-1H-pyrrolo[3,2-c]pyridin-6-yl)benzamide;
4-(3-(4-(2-(2,6-dioxopiperidin-3-yl)-1-oxoisoindolin-4-yl)piperazin-1-yl)propyl)-N-(2-((R)-1-methylpyrrolidin-2-yl)-1H-pyrrolo[3,2-c]pyridin-6-yl)benzamide;
4-((2S)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide;
4-((2R)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide;
4-((R)-4-((1-(2-((rel-R)-2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide;
4-((R)-4-((1-(2-((rel-S)-2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide;
4-(4-((1-(2-(2,6-dioxopiperidin-3-yl)-1,3-dioxoisoindolin-4-yl)piperidin-4-yl)oxy)piperidin-1-yl)-N-(2-((R)-1-methylpyrrolidin-2-yl)-1H-pyrrolo[3,2-c]pyridin-6-yl)benzamide;
4-(4-((1-(2-(2,6-dioxopiperidin-3-yl)-1-oxoisoindolin-4-yl)piperidin-4-yl)oxy)piperidin-1-yl)-N-(2-((R)-1-methylpyrrolidin-2-yl)-1H-pyrrolo[3,2-c]pyridin-6-yl)benzamide;
6-(4-(2-(4-(2-(2,6-dioxopiperidin-3-yl)-1-oxoisoindolin-4-yl)piperazin-1-yl)ethyl)piperidin-1-yl)-N-(2-((R)-1-methylpyrrolidin-2-yl)-1H-pyrrolo[3,2-c]pyridin-6-yl)nicotinamide;
6-(4-((1-(2-(2,6-dioxopiperidin-3-yl)-1-oxoisoindolin-5-yl)piperidin-4-yl)oxy)piperidin-1-yl)-N-(2-((R)-1-methylpyrrolidin-2-yl)-1H-pyrrolo[3,2-c]pyridin-6-yl)nicotinamide;
6-(4-(((1-(2-(2,6-dioxopiperidin-3-yl)-1-oxoisoindolin-4-yl)piperidin-4-yl)oxy)methyl)piperidin-1-yl)-N-(2-((R)-1-methylpyrrolidin-2-yl)-1H-pyrrolo[3,2-c]pyridin-6-yl)nicotinamide;
4-(2-(4-(2-(2,6-dioxopiperidin-3-yl)-1-oxoisoindolin-4-yl)piperidin-1-yl)ethoxy)-N-(2-((R)-1-methylpyrrolidin-2-yl)-1H-pyrrolo[3,2-c]pyridin-6-yl)benzamide;
4-(2-(4-(2-(2,6-dioxopiperidin-3-yl)-1-oxoisoindolin-4-yl)piperazin-1-yl)ethoxy)-N-(2-((R)-1-methylpyrrolidin-2-yl)-1H-pyrrolo[3,2-c]pyridin-6-yl)benzamide;
4-((1-((1-(2-(2,6-dioxopiperidin-3-yl)-1-oxoisoindolin-5-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
4-((1-((1-(2-(2,6-dioxopiperidin-3-yl)-1,3-dioxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
6-(4-((1-(2-(2,6-dioxopiperidin-3-yl)-1-oxoisoindolin-4-yl)piperidin-4-yl)oxy)piperidin-1-yl)-N-(2-((R)-1-methylpyrrolidin-2-yl)-1H-pyrrolo[3,2-c]pyridin-6-yl)nicotinamide;
4-(4-((1-(2-(2,6-dioxopiperidin-3-yl)-1-oxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)benzamide;
6-(4-((1-(2-(2,6-dioxopiperidin-3-yl)-1-oxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)nicotinamide;
6-[4-[[1-[2-(2,6-dioxo-3-piperidyl)-1,3-dioxo-isoindolin-4-yl]-4-piperidyl]oxy]-1-piperidyl]-N-[2-[(2R)-1-methylpyrrolidin-2-yl]-1H-pyrrolo[3,2-c]pyridin-6-yl]pyridine-3-carboxamide;
6-(4-(5-(2-(2,6-dioxopiperidin-3-yl)-1-oxoisoindolin-4-yl)pent-4-yn-1-yl)piperazin-1-yl)-N-(2-((R)-1-methylpyrrolidin-2-yl)-1H-pyrrolo[3,2-c]pyridin-6-yl)nicotinamide;
6-((5-(2-(2,6-dioxopiperidin-3-yl)-1-oxoisoindolin-4-yl)pent-4-yn-1-yl)oxy)-N-(2-((R)-1-methylpyrrolidin-2-yl)-1H-pyrrolo[3,2-c]pyridin-6-yl)nicotinamide;
4-(4-((1-(2-(2,6-dioxopiperidin-3-yl)-1,3-dioxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)benzamide;
6-(1′-(2-(2,6-dioxopiperidin-3-yl)-1,3-dioxoisoindolin-4-yl)-[4,4′-bipiperidin]-1-yl)-N-(2-((R)-1-methylpyrrolidin-2-yl)-1H-pyrrolo[3,2-c]pyridin-6-yl)nicotinamide;
6-(4-(3-(2-(2,6-dioxopiperidin-3-yl)-1-oxoisoindolin-4-yl)prop-2-yn-1-yl)piperidin-1-yl)-N-(2-((R)-1-methylpyrrolidin-2-yl)-1H-pyrrolo[3,2-c]pyridin-6-yl)pyridazine-3-carboxamide;
5-(4-(3-(2-(2,6-dioxopiperidin-3-yl)-1-oxoisoindolin-4-yl)prop-2-yn-1-yl)piperidin-1-yl)-N-(2-((R)-1-methylpyrrolidin-2-yl)-1H-pyrrolo[3,2-c]pyridin-6-yl)pyrazine-2-carboxamide;
4-(4-(3-(2-(2,6-dioxopiperidin-3-yl)-1-oxoisoindolin-4-yl)prop-2-yn-1-yl)piperazin-1-yl)-2-fluoro-N-(2-((R)-1-methylpyrrolidin-2-yl)-1H-pyrrolo[3,2-c]pyridin-6-yl)benzamide;
4-(6-(2-(2,6-dioxopiperidin-3-yl)-1-oxoisoindolin-4-yl)hex-5-yn-1-yl)-3,5-difluoro-N-(2-((R)-1-methylpyrrolidin-2-yl)-1H-pyrrolo[3,2-c]pyridin-6-yl)benzamide;
4-((1-((1-(2-(2,6-dioxopiperidin-3-yl)-1,3-dioxoisoindolin-5-yl)piperidin-4-yl)methyl)azetidin-3-yl)oxy)-N-(2-((R)-1-methylpyrrolidin-2-yl)-1H-pyrrolo[3,2-c]pyridin-6-yl)benzamide;
6-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)nicotinamide;
4-((1-(2-(2,6-dioxopiperidin-3-yl)-1-oxoisoindolin-4-yl)piperidin-4-yl)methoxy)-N-(2-((R)-1-methylpyrrolidin-2-yl)-1H-pyrrolo[3,2-c]pyridin-6-yl)benzamide;
N-(3-chloro-2-((R)-1-methylpyrrolidin-2-yl)-1H-pyrrolo[3,2-c]pyridin-6-yl)-4-(2-(1-(2-(2,6-dioxopiperidin-3-yl)-1-oxoisoindolin-4-yl)piperidin-4-yl)ethoxy)benzamide;
N-(3-chloro-2-((R)-1-methylpyrrolidin-2-yl)-1H-pyrrolo[3,2-c]pyridin-6-yl)-4-(2-(1-(2-((rel-S)-2,6-dioxopiperidin-3-yl)-1-oxoisoindolin-4-yl)piperidin-4-yl)ethoxy)benzamide;
N-(3-chloro-2-((R)-1-methylpyrrolidin-2-yl)-1H-pyrrolo[3,2-c]pyridin-6-yl)-4-(2-(1-(2-((rel-R)-2,6-dioxopiperidin-3-yl)-1-oxoisoindolin-4-yl)piperidin-4-yl)ethoxy)benzamide;
6-((2R)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)nicotinamide;
4-(2-(9-(2-(2,6-dioxopiperidin-3-yl)-1-oxoisoindolin-5-yl)-3,9-diazaspiro[5.5]undecan-3-yl)ethoxy)-N-(2-((R)-1-methylpyrrolidin-2-yl)-1H-pyrrolo[3,2-c]pyridin-6-yl)benzamide;
4-(2-(9-(2-(2,6-dioxopiperidin-3-yl)-1-oxoisoindolin-4-yl)-3,9-diazaspiro[5.5]undecan-3-yl)ethoxy)-N-(2-((R)-1-methylpyrrolidin-2-yl)-1H-pyrrolo[3,2-c]pyridin-6-yl)benzamide;
4-(2-(9-(2-(2,6-dioxopiperidin-3-yl)-1,3-dioxoisoindolin-5-yl)-3,9-diazaspiro[5.5]undecan-3-yl)ethoxy)-N-(2-((R)-1-methylpyrrolidin-2-yl)-1H-pyrrolo[3,2-c]pyridin-6-yl)benzamide;
4-(1-((1-(2-(2,6-dioxopiperidin-3-yl)-1-oxoisoindolin-4-yl)piperidin-4-yl)methyl)piperidin-4-yl)-N-(2-((R)-1-methylpyrrolidin-2-yl)-1H-pyrrolo[3,2-c]pyridin-6-yl)benzamide;
6-(4-((1-(2-(2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)nicotinamide;
4-(4-((1-(1-(2,6-dioxopiperidin-3-yl)-3-methyl-2-oxo-2,3-dihydro-1H-benzo[d]imidazol-5-yl)piperidin-4-yl)methyl)piperazin-1-yl)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide;
4-(2-(1-(2-((rel-S)-2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide;
4-(2-(1-(2-((rel-R)-2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide;
4-(3-(1-(2-(2,6-dioxopiperidin-3-yl)-1-oxoisoindolin-4-yl)piperidin-4-yl)propyl)-2-fluoro-N-(2-((R)-1-methylpyrrolidin-2-yl)-1H-pyrrolo[3,2-c]pyridin-6-yl)benzamide;
4-(3-(1-(2-((rel-S)-2,6-dioxopiperidin-3-yl)-1-oxoisoindolin-4-yl)piperidin-4-yl)propyl)-2-fluoro-N-(2-((R)-1-methylpyrrolidin-2-yl)-1H-pyrrolo[3,2-c]pyridin-6-yl)benzamide;
4-(3-(1-(2-((rel-R)-2,6-dioxopiperidin-3-yl)-1-oxoisoindolin-4-yl)piperidin-4-yl)propyl)-2-fluoro-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1-oxoisoindolin-4-yl)piperidin-4-yl)ethyl)-N-(2-((R)-1-methylpyrrolidin-2-yl)-1H-pyrrolo[3,2-c]pyridin-6-yl)benzamide;
6-(4-(4-(2-(2,6-dioxopiperidin-3-yl)-1,3-dioxoisoindolin-4-yl)phenyl)piperidin-1-yl)-N-(2-((R)-1-methylpyrrolidin-2-yl)-1H-pyrrolo[3,2-c]pyridin-6-yl)nicotinamide;
6-(2-(1-(2-(2,6-dioxopiperidin-3-yl)-1,3-dioxoisoindolin-4-yl)piperidin-4-yl)ethoxy)-N-(2-((R)-1-methylpyrrolidin-2-yl)-1H-pyrrolo[3,2-c]pyridin-6-yl)nicotinamide;
4-(4-((1-(2-(2,6-dioxopiperidin-3-yl)-6-fluoro-1,3-dioxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide;
N—((S)-2,6-dioxopiperidin-3-yl)-5-(4-((4-(4-((2-((R)-1-methylpyrrolidin-2-yl)-1H-pyrrolo[3,2-c]pyridin-6-yl)carbamoyl)phenyl)piperazin-1-yl)methyl)piperidin-1-yl)picolinamide;
N—((S)-2,6-dioxopiperidin-3-yl)-6-(4-((4-(4-((2-((R)-1-methylpyrrolidin-2-yl)-1H-pyrrolo[3,2-c]pyridin-6-yl)carbamoyl)phenyl)piperazin-1-yl)methyl)piperidin-1-yl)picolinamide;
4-(3-((4-(2-(2,6-dioxopiperidin-3-yl)-1,3-dioxoisoindolin-5-yl)piperazin-1-yl)methyl)azetidin-1-yl)-N-(2-((R)-1-methylpyrrolidin-2-yl)-1H-pyrrolo[3,2-c]pyridin-6-yl)benzamide;
4-((1-((1-(2-(2,6-dioxopiperidin-3-yl)-6-fluoro-1,3-dioxoisoindolin-5-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
6-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)pyridazine-3-carboxamide;
5-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)pyrazine-2-carboxamide;
6-((3S)-3-(3-(2-(2,6-dioxopiperidin-3-yl)-1-oxoisoindolin-4-yl)prop-2-yn-1-yl)pyrrolidin-1-yl)-N-(2-((R)-1-methylpyrrolidin-2-yl)-1H-pyrrolo[3,2-c]pyridin-6-yl)nicotinamide;
6-(4-(6-(2-(2,6-dioxopiperidin-3-yl)-1-oxoisoindolin-4-yl)hex-5-yn-1-yl)piperidin-1-yl)-N-(2-((R)-1-methylpyrrolidin-2-yl)-1H-pyrrolo[3,2-c]pyridin-6-yl)nicotinamide;
4-(4-(3-(1-(2-(2,6-dioxopiperidin-3-yl)-1-oxoisoindolin-4-yl)piperidin-4-yl)propanoyl)piperazin-1-yl)-N-(2-((R)-1-methylpyrrolidin-2-yl)-1H-pyrrolo[3,2-c]pyridin-6-yl)benzamide;
N-(3-chloro-2-((R)-1-methylpyrrolidin-2-yl)-1H-pyrrolo[3,2-c]pyridin-6-yl)-4-(2-(1-(2-(2,6-dioxopiperidin-3-yl)-1-oxoisoindolin-4-yl)piperidin-4-yl)ethoxy)-2-fluorobenzamide;
N-(3-chloro-2-((R)-1-methylpyrrolidin-2-yl)-1H-pyrrolo[3,2-c]pyridin-6-yl)-6-(4-((1-(2-(2,6-dioxopiperidin-3-yl)-1-oxoisoindolin-4-yl)piperidin-4-yl)methoxy)piperidin-1-yl)nicotinamide;
3-(4-((1-(2-(2,6-dioxopiperidin-3-yl)-1-oxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide;
6-(4-(2-(1-(2-(2,6-dioxopiperidin-3-yl)-1-oxoisoindolin-4-yl)piperidin-4-yl)ethyl)piperazin-1-yl)-N-(2-((R)-1-methylpyrrolidin-2-yl)-1H-pyrrolo[3,2-c]pyridin-6-yl)nicotinamide;
4-((4-((1-(2-(2,6-dioxopiperidin-3-yl)-1-oxoisoindolin-4-yl)piperidin-4-yl)methyl)piperazin-1-yl)methyl)-N-(2-((R)-1-methylpyrrolidin-2-yl)-1H-pyrrolo[3,2-c]pyridin-6-yl)benzamide;
6-(4-((3-(2-(2,6-dioxopiperidin-3-yl)-1-oxoisoindolin-4-yl)propa-1,2-dien-1-yl)oxy)piperidin-1-yl)-N-(2-((R)-1-methylpyrrolidin-2-yl)-1H-pyrrolo[3,2-c]pyridin-6-yl)nicotinamide;
6-(4-(4-(2-(2,6-dioxopiperidin-3-yl)-1-oxoisoindolin-4-yl)-3-methylphenoxy)piperidin-1-yl)-N-(2-((R)-1-methylpyrrolidin-2-yl)-1H-pyrrolo[3,2-c]pyridin-6-yl)nicotinamide;
5-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)picolinamide;
4-(3-(4-(2-(2,6-dioxopiperidin-3-yl)-1-oxoisoindolin-4-yl)piperidin-1-yl)propyl)-N-(2-((R)-1-methylpyrrolidin-2-yl)-1H-pyrrolo[3,2-c]pyridin-6-yl)benzamide;
4-(3-(4-(2-(2,6-dioxopiperidin-3-yl)-1-oxoisoindolin-4-yl)phenyl)propyl)-N-(2-((R)-1-methylpyrrolidin-2-yl)-1H-pyrrolo[3,2-c]pyridin-6-yl)benzamide;
4-((7-(2-(2,6-dioxopiperidin-3-yl)-1-oxoisoindolin-4-yl)-7-azaspiro[3.5]nonan-2-yl)oxy)-2-fluoro-N-(2-((R)-1-methylpyrrolidin-2-yl)-1H-pyrrolo[3,2-c]pyridin-6-yl)benzamide;
4-((7-(2-(2,6-dioxopiperidin-3-yl)-1-oxoisoindolin-4-yl)-7-azaspiro[3.5]nonan-2-yl)methoxy)-2-fluoro-N-(2-((R)-1-methylpyrrolidin-2-yl)-1H-pyrrolo[3,2-c]pyridin-6-yl)benzamide;
4-(9-(2-(2,6-dioxopiperidin-3-yl)-1-oxoisoindolin-4-yl)-3,9-diazaspiro[5.5]undecan-3-yl)-2-fluoro-N-(2-((R)-1-methylpyrrolidin-2-yl)-1H-pyrrolo[3,2-c]pyridin-6-yl)benzamide;
4-(4-(4-(2-(2,6-dioxopiperidin-3-yl)-1-oxoisoindolin-4-yl)-3-methylphenoxy)piperidin-1-yl)-2-fluoro-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1-oxoisoindolin-4-yl)piperidin-4-yl)ethoxy)-2-methyl-N-(2-((R)-1-methylpyrrolidin-2-yl)-1H-pyrrolo[3,2-c]pyridin-6-yl)benzamide;
2-chloro-4-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1-oxoisoindolin-4-yl)piperidin-4-yl)ethoxy)-3-fluoro-N-(2-((R)-1-methylpyrrolidin-2-yl)-1H-pyrrolo[3,2-c]pyridin-6-yl)benzamide;
4-(4-(2-(2,6-dioxopiperidin-3-yl)-1-oxoisoindolin-4-yl)phenethoxy)-2-fluoro-N-(2-((R)-1-methylpyrrolidin-2-yl)-1H-pyrrolo[3,2-c]pyridin-6-yl)benzamide;
4-(4-(2-(2,6-dioxopiperidin-3-yl)-1-oxoisoindolin-4-yl)-3-methylphenethoxy)-2-fluoro-N-(2-((R)-1-methylpyrrolidin-2-yl)-1H-pyrrolo[3,2-c]pyridin-6-yl)benzamide;
4-(3-(1-(2-(2,6-dioxopiperidin-3-yl)-1-oxoisoindolin-4-yl)piperidin-4-yl)propoxy)-2-fluoro-N-(2-((R)-1-methylpyrrolidin-2-yl)-1H-pyrrolo[3,2-c]pyridin-6-yl)benzamide;
4-(1-(3-(1-(2-(2,6-dioxopiperidin-3-yl)-1,3-dioxoisoindolin-4-yl)piperidin-4-yl)propyl)-1H-pyrazol-4-yl)-2-fluoro-N-(2-((R)-1-methylpyrrolidin-2-yl)-1H-pyrrolo[3,2-c]pyridin-6-yl)benzamide;
5-(1-(3-(1-(2-(2,6-dioxopiperidin-3-yl)-1,3-dioxoisoindolin-4-yl)piperidin-4-yl)propyl)-1H-pyrazol-4-yl)-N-(2-((R)-1-methylpyrrolidin-2-yl)-1H-pyrrolo[3,2-c]pyridin-6-yl)picolinamide;
4-(1-(3-(1-(2-(2,6-dioxopiperidin-3-yl)-1-oxoisoindolin-4-yl)piperidin-4-yl)propyl)-1H-pyrazol-4-yl)-2-fluoro-N-(2-((R)-1-methylpyrrolidin-2-yl)-1H-pyrrolo[3,2-c]pyridin-6-yl)benzamide;
4-((5-(2-(2,6-dioxopiperidin-3-yl)-1-oxoisoindolin-4-yl)pent-4-yn-1-yl)oxy)-2-fluoro-N-(2-((R)-1-methylpyrrolidin-2-yl)-1H-pyrrolo[3,2-c]pyridin-6-yl)benzamide;
4-((1-(2-(2,6-dioxopiperidin-3-yl)-1-oxoisoindolin-4-yl)piperidin-4-yl)methoxy)-2-fluoro-N-(2-((R)-1-methylpyrrolidin-2-yl)-1H-pyrrolo[3,2-c]pyridin-6-yl)benzamide;
N—((S)-2,6-dioxopiperidin-3-yl)-2-fluoro-4-(4-(2-(3-fluoro-4-((2-((R)-1-methylpyrrolidin-2-yl)-1H-pyrrolo[3,2-c]pyridin-6-yl)carbamoyl)phenoxy)ethyl)piperidin-1-yl)benzamide;
5-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)picolinamide;
4-(2-(1-(2-(2,6-dioxopiperidin-3-yl)-1,3-dioxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide;
4-(4-(4-(2-(2,6-dioxopiperidin-3-yl)-1-oxoisoindolin-4-yl)phenyl)piperidin-1-yl)-2-fluoro-N-(2-((R)-1-methylpyrrolidin-2-yl)-1H-pyrrolo[3,2-c]pyridin-6-yl)benzamide;
N—((S)-2,6-dioxopiperidin-3-yl)-2-fluoro-3-(5-(4-((2-((R)-1-methylpyrrolidin-2-yl)-1H-pyrrolo[3,2-c]pyridin-6-yl)carbamoyl)phenyl)pent-1-yn-1-yl)benzamide;
3-(4-((1-(2-(2,6-dioxopiperidin-3-yl)-1,3-dioxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide;
4-(4-((1-(2-(2,6-dioxopiperidin-3-yl)-1,3-dioxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide;
4-(4-((1-(2-(2,6-dioxopiperidin-3-yl)-1-oxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide;
4-(6-(2-(2,6-dioxopiperidin-3-yl)-1-oxoisoindolin-5-yl)hex-5-yn-1-yl)-2-fluoro-N-(2-((R)-1-methylpyrrolidin-2-yl)-1H-pyrrolo[3,2-c]pyridin-6-yl)benzamide;
6-(4-(3-(2-(2,6-dioxopiperidin-3-yl)-1-oxoisoindolin-5-yl)prop-2-yn-1-yl)piperidin-1-yl)-N-(2-((R)-1-methylpyrrolidin-2-yl)-1H-pyrrolo[3,2-c]pyridin-6-yl)nicotinamide;
4-(5-(2-(2,6-dioxopiperidin-3-yl)-3-oxoisoindolin-5-yl)pent-4-yn-1-yl)-N-(2-((R)-1-methylpyrrolidin-2-yl)-1H-pyrrolo[3,2-c]pyridin-6-yl)benzamide;
6-((3R)-3-(3-(2-(2,6-dioxopiperidin-3-yl)-1-oxoisoindolin-4-yl)prop-2-yn-1-yl)pyrrolidin-1-yl)-N-(2-((R)-1-methylpyrrolidin-2-yl)-1H-pyrrolo[3,2-c]pyridin-6-yl)nicotinamide;
4-((1-(2-(1-(2-(2,6-dioxopiperidin-3-yl)-1,3-dioxoisoindolin-5-yl)piperidin-4-yl)ethyl)azetidin-3-yl)oxy)-N-(2-((R)-1-methylpyrrolidin-2-yl)-1H-pyrrolo[3,2-c]pyridin-6-yl)benzamide;
6-(4-((1-(2-(2,6-dioxopiperidin-3-yl)-1-oxoisoindolin-5-yl)piperidin-4-yl)methoxy)piperidin-1-yl)-N-(2-((R)-1-methylpyrrolidin-2-yl)-1H-pyrrolo[3,2-c]pyridin-6-yl)nicotinamide;
N-(3-chloro-2-((R)-1-methylpyrrolidin-2-yl)-1H-pyrrolo[3,2-c]pyridin-6-yl)-4-(2-(1-(2-(2,6-dioxopiperidin-3-yl)-1-oxoisoindolin-4-yl)piperidin-4-yl)ethoxy)-3-fluorobenzamide;
4-(2-(1-(2-(2,6-dioxopiperidin-3-yl)-1-oxoisoindolin-4-yl)piperidin-4-yl)ethoxy)-2,5-difluoro-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1-oxoisoindolin-4-yl)piperidin-4-yl)ethoxy)-2,3-difluoro-N-(2-((R)-1-methylpyrrolidin-2-yl)-1H-pyrrolo[3,2-c]pyridin-6-yl)benzamide;
4-(3-(1-(2-((S)-2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide; and
N-[3-chloro-2-[(R)-1-methylpyrrolidin-2-yl]-1H-pyrrolo[3,2-c]pyridin-6-yl]-4-[3-[1-[2-[(rel-S)-2,6-dioxo-3-piperidyl]-1-oxo-isoindolin-4-yl]-4-piperidyl]propyl]benzamide;
   and the pharmaceutically acceptable salts thereof.

### Therapeutic Efficacy

The compounds of the present invention are therapeutically useful. The present invention therefore provides compounds as described herein, for use in medicine. The present invention provides compounds as described herein, for use in treating the human or animal body. For the avoidance of doubt, the agent may comprise a compound of the invention in the form of a solvate. Also provided is a pharmaceutical composition comprising a compound of the invention together with a pharmaceutically acceptable carrier or diluent. Typically, the composition contains up to 85 wt % of a compound of the invention. More typically, it contains up to 50 wt % of a compound of the invention. Preferred pharmaceutical compositions are sterile and pyrogen free. Further, when the pharmaceutical compositions provided by the invention contain a compound of the invention which is optically active, the compound of the invention is typically a substantially pure optical isomer.

The composition of the invention may be provided as a kit comprising instructions to enable the kit to be used in the methods described herein or details regarding which subjects the method may be used for.

As explained above, the compounds of the invention are useful in treating or preventing various disorders. Disorders for treatment using the compounds of the invention may include cancer. In particular, the compounds of the invention are useful for inducing selective degradation of MLLT1 and/or MLLT3, and/or inhibiting MLLT1 and/or MLLT3.

Cancer, e.g. acute lymphoblastic leukemia, acute myeloid leukemia, adrenocortical carcinoma, aids-related lymphoma, primary CNS lymphoma, anal cancer, astrocytomas, brain cancer, basal cell carcinoma, bile duct cancer, bladder cancer, bone cancer (e.g. ewing sarcoma, osteosarcoma and malignant fibrous histiocytoma), breast cancer, bronchial tumors, medulloblastoma and other CNS embryonal tumors, cervical cancer, chronic lymphocytic leukemia, chronic myelogenous leukemia, chronic myeloproliferative neoplasms, colorectal cancer, craniopharyngioma, endometrial cancer, ependymoma, esophageal cancer, esthesioneuroblastoma, ewing sarcoma, extragonadal germ cell tumor, intraocular melanoma, retinoblastoma, fallopian tube cancer, gallbladder cancer, gastric cancer, gastrointestinal carcinoid tumor, gastrointestinal stromal tumors (gist), germ cell tumors, extragonadal germ cell tumors, ovarian germ cell tumors, testicular cancer, gestational trophoblastic disease, hairy cell leukemia, hepatocellular cancer, histiocytosis, langerhans cell, hodgkin lymphoma, hypopharyngeal cancer, intraocular melanoma, islet cell tumors, pancreatic neuroendocrine tumors, kaposi sarcoma, kidney (renal cell) cancer, langerhans cell histiocytosis, laryngeal cancer, leukemia, liver cancer, lung cancer (non-small cell, small cell, pleuropulmonary blastoma, and tracheobronchial tumor), lymphoma, malignant fibrous histiocytoma of bone and osteosarcoma, merkel cell carcinoma, mesothelioma, mouth cancer, multiple endocrine neoplasia syndromes, multiple myeloma/plasma cell neoplasms, mycosis fungoides, myelodysplastic syndromes, myelodysplastic/myeloproliferative neoplasms, myelogenous leukemia, neuroblastoma, non-hodgkin lymphoma, oropharyngeal cancer, osteosarcoma and undifferentiated pleomorphic sarcoma, pancreatic cancer, pancreatic neuroendocrine tumors (islet cell tumors), papillomatosis, paraganglioma, parathyroid cancer, penile cancer, pharyngeal cancer, pheochromocytoma, pituitary tumor, prostate cancer, rectal cancer, retinoblastoma, rhabdomyosarcoma, t-cell lymphoma, testicular cancer, throat cancer, thymoma and thymic carcinoma, thyroid cancer, tracheobronchial tumors and the like is particularly suitable to being treated with the compounds of the invention provided herein.

Typically, the compounds of the invention are for use in treating cancers which are transcriptionally addicted cancers, such as breast cancer, acute leukemia, chronic leukemia, prostate cancer, bladder cancer, cholangiocarcinoma, colon adenocarcinoma, esophageal carcinoma, head and neck squamous cell carcinoma, kidney chromophobe, kidney renal clear cell carcinoma, kidney renal papillary cell carcinoma, liver hepatocellular carcinoma, lung adenocarcinoma, pheochromocytoma and paraganglioma, rectum adenocarcinoma, stomach adenocarcinoma, uterine corpus endometrial carcinoma, cervical squamous cell carcinoma and endocervical adenocarcinoma, lung squamous cell carcinoma and sarcoma (Cell Rep, 2021, 16, 1087).

Preferably, the compounds of the invention are for use in treating acute myeloid leukaemia (AML) or acute lymphoblastic leukaemia (ALL), such as mixed-lineage leukaemia (MLL) rearranged acute leukemia (AML and ALL), NPM1 mutant acute leukemia, RUNX1-fusion acute leukemia, E2A-fusion acute leukemia, PML-fusion acute leukemia or NUP98-fusion acute leukemia.

The compounds of the invention may be used as standalone therapeutic agents. Alternatively, they may be used in combination with other active agents such as chemotherapeutic agents, targeted precision medicines or immune oncology agents. For example, they may be used in combination with a menin inhibitor (for example revumenib, ziftomenib, bleximenib), Bcl2 targeted drug (for instance venetoclax), FLT3 inhibitor (for instance sorafenib, quizartinib or gilteritinib), IDH inhibitor (for instance enasideniob), EGFR inhibitor (for instance erlotinib, gefitinib, lapatinib or osimertinib), CDK4/6 inhibitor (for instance abemaciclib, palbociclib or ribociclib), a chemotherapy (for instance cytarabine, daunorubicin, azacitidine, cedazuridine, all trans retinoic acid, doxorubicin, gemcitabine, cisplatin or paclitaxel) or an immune checkpoint inhibitor (for instance pembrolizumab, nivolumab, durvalumab or cemiplimab).

When used to treat a cancer, the compounds of the invention may be used in alleviating, ameliorating or preventing aggravation of the symptoms of the cancer. Typically, treating a cancer may comprise reducing progression of the cancer, e.g. increasing progression free survival (PFS) and/or increasing survival e.g. increasing overall survival (OS). Treating a cancer may comprise preventing or inhibiting growth of a tumour associated with the cancer. Treating a cancer may comprise preventing metastasis of the cancer. Preferably, treating a cancer may comprise reducing the size of a tumour associated with the cancer. As such, the treatment may cause tumour regression in the cancer. Treating a cancer may comprise reducing the number of tumours or lesions present in the patient. When the treatment reduces the size of a tumour associated with the cancer, the size of the tumour is typically reduced from base line by at least 10%. Base line is the size of the tumour at the date treatment with the compound is first started. The size of the tumour is typically as measured in accordance with version 1.1 of the RECIST criteria (for instance as described in Eisenhauer et al, European Journal of Cancer 45 (2009) 228-247).

The response to the treatment with the compound may be complete response, partial response or stable disease, in accordance with version 1.1 of the RECIST criteria. Preferably, the response is partial response or complete response. The treatment may achieve progression free survival for at least 60 days, at least 120 days or at least 180 days.

The reduction in tumour size may be greater 20%, greater than 30% or greater than 50% reduction relative to base line. The reduction in tumour size may be observed after 30 days of treatment or after 60 days of treatment.

In haematological cancers, treating a cancer preferentially may lead to complete remission (CR) of the cancer or it may lead to complete remission with incomplete haematological recovery (CRh) or complete remission but with incomplete platelet recovery (CRp). Additionally, treatment may lead to an increase in duration of remission (DoR), an increase in minimal residual disease (MRD), or an increase in relapse free survival (RFS) (Blood 2007 109 1815 and Leukemia Res 2018, 68, 32).

As explained here, the compounds of the invention are useful in treating or preventing various disorders. The present invention therefore provides a compound of the invention for use in medicine. The invention also provides the use of a compound of the invention in the manufacture of a medicament. The invention also provides compositions and products comprising the compounds of the invention. Such compositions and products are also useful in treating or preventing disorders. The present invention therefore provides a composition or product as defined herein for use in medicine. The invention also provides the use of a composition or product of the invention in the manufacture of a medicament. Also provided is a method of treating a subject in need of such treatment, said method comprising administering to the subject a compound of the invention. In some embodiments the subject suffers from or is at risk of suffering from one of the disorders disclosed herein.

In one aspect, the subject is a mammal, in particular a human. However, it may be non-human. Preferred non-human animals include, but are not limited to, primates, such as marmosets or monkeys, commercially farmed animals, such as horses, cows, sheep or pigs, and pets, such as dogs, cats, mice, rats, guinea pigs, ferrets, gerbils or hamsters. The subject can be any animal that is capable of being infected by a bacterium.

A subject is typically a human patient. The patient may be male or female. The age of the patient is typically at least 18 years, for instance from 30 to 70 years or from 40 to 60 years. Alternatively, the patient may be a child, for instance the patient may be under 18 years of age, or under 12 years of age. In one embodiment, the patient is a child under ten years of age or an infant under two years of age. In one embodiment the invention provides a compound as defined herein for use in treating cancer in paediatric patients.

A compound or composition of the invention can be administered to the subject in order to treat one or more symptoms of the disorder. In this embodiment, the subject is typically symptomatic. A therapeutically effective amount of the agent or formulation is administered to such a subject. A therapeutically effective amount is an amount effective to ameliorate one or more symptoms of the disorder.

The compound or composition of the invention may be administered in a variety of dosage forms. Thus, it can be administered orally, for example as tablets, troches, lozenges, aqueous or oily suspensions, dispersible powders or granules. The compound or composition of the invention may also be administered parenterally, whether subcutaneously, intravenously, intramuscularly, intrasternally, transdermally or by infusion techniques. The compound or composition may also be administered as a suppository. Preferably, the compound, composition or combination may be administered orally.

The compound or composition of the invention is typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, tale, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections or inhalation may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for inhalation, injection or infusion may contain as carrier, for example, sterile water or preferably they may be in the form of sterile, aqueous, isotonic saline solutions. Pharmaceutical compositions suitable for delivery by needleless injection, for example, transdermally, may also be used.

A therapeutically effective amount of the compound or composition of the invention is administered to a subject. The dose may be determined according to various parameters, especially according to the compound used; the age, weight and condition of the subject to be treated; the route of administration; and the required regimen. Again, a physician will be able to determine the required route of administration and dosage for any particular subject. A typical daily dose is from about 0.01 to 100 mg per kg, preferably from about 0.1 mg/kg to 50 mg/kg, e.g. from about 1 to 10 mg/kg of body weight, according to the activity of the specific compound, the age, weight and conditions of the subject to be treated, the type and severity of the disease and the frequency and route of administration. Preferably, daily dosage levels are from 5 mg to 2 g.

When the compound or composition of the invention is administered to a subject in combination with another active agent, the dose of the other active agent can be determined as described above. The dose may be determined according to various parameters, especially according to the agent used; the age, weight and condition of the subject to be treated; the route of administration; and the required regimen. Again, a physician will be able to determine the required route of administration and dosage for any particular subject. A typical daily dose is from about 0.01 to 100 mg per kg, preferably from about 0.1 mg/kg to 50 mg/kg, e.g. from about 1 to 10 mg/kg of body weight, according to the activity of the specific agent, the age, weight and conditions of the subject to be treated, the type and severity of the disease and the frequency and route of administration. Preferably, daily dosage levels are from 5 mg to 2 g.

### Synthesis

Compounds of the invention can be prepared by the synthetic methods described in the Examples that follow, or by analogy with such methods using appropriate starting materials and methodologies familiar to the skilled chemist.

The following examples illustrate the invention. They do not however limit the invention in any way. In this regard, it is important to understand that the particular assay used in the Examples section is designed only to provide an indication of biological activity. There are many assays available to determine biological activity, and a negative result in any one particular assay is therefore not determinative. The invention is defined according to the claims.

### EXAMPLES

In the examples which follow below, compounds may be produced in racemic form. Compounds in racemic form are indicated by use of the term rac- in advance of the IUPAC name. In some instances, as indicated, enantiomers and/or diastereomers are separated. In general, the enantiomers and/or diastereomers of the invention can be separated using chiral chromatography. The separated enantiomers and/or diastereomers may be identified in the order in which they elute, for example the first, second, third or fourth eluting isomer respectively, as obtained by separation using chiral chromatography. The isomers may alternatively or additionally be identified using IUPAC naming conventions. Thus, where enantiomers and/or diastereomers have been assigned an absolute stereochemistry, this is indicated as R or S stereochemistry, as appropriate, and by use of a wedge-shaped bond (e.g. [symbol] )in the chemical formula.

Where enantiomers have been separated but have undefined absolute stereochemistry the term “rel-R” or “rel-S” has been used. In these examples it is understood that the absolute configuration is unknown and the “rel-R” and “rel-S” labels are used merely to distinguish the two enantiomers form one another. The absolute stereochemistry of an enantiomer labelled as “rel-R” may be either R or S. In either case, the corresponding “rel-S” enantiomer has the opposite configuration to the “rel-R” enantiomer. In these examples the use of a rectangular bond (e.g. [symbol] ) has been used to define the “rel” configuration. This labelling convention also applies where diastereomers have been separated and the absolute configuration of a first chiral centre is known but the absolute configuration of a second chiral centre is unknown. In these examples, the known absolute configuration of the first chiral centre is indicated as R or S stereochemistry, as appropriate, and by use of a wedge-shaped bond (e.g [symbol] ) in the chemical formula, while the unknown absolute configuration of the second chiral centre is indicated as “rel-R” or “rel-S”, and by use of a rectangular bond (e.g. [symbol] ) in the chemical formula.

Alternatively, where diastereomers have been separated and the absolute configuration of both chiral centres is unknown they may be assigned a relative stereochemistry, which denotes the stereochemistry of a first chiral centre with respect to a second chiral centre. Where a relative stereochemistry has been assigned, this is indicated by the term “rel” in the IUPAC name, and by use of a rectangular-shaped bond (e.g. [symbol] ). Where diastereomers have been separated and the relative and absolute configuration is unknown, the compounds have been labelled by the order in which they elute, for example the first, second, third or fourth eluting isomer respectively, as obtained by separation using chiral chromatography.

The moieties U of formulae (U1), (U4) when Q′ is CH and (U5) typically contain at least one chiral centre at the carbon atoms marked with an asterisk as depicted below:

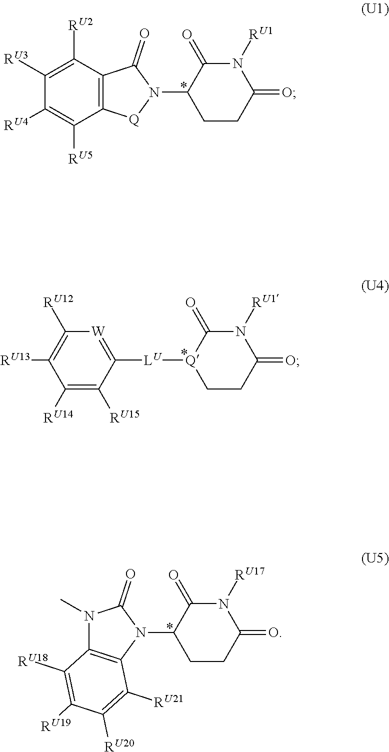

Specifically in relation to the examples that follow, unless otherwise stated, it is understood that the moiety U is typically in the form of a 1:1 mixture of the R- and S-enantiomers at said carbon atom, indicated specifically in the examples that follow by the use of a simple line-shaped bond (e.g. [symbol] ). In certain examples, the enantiomers or diastereomers have been separated such that the compounds are in enantiomerically or diastereomerically pure form. In these examples, when the stereochemistry at the chiral carbon atom in moiety U is known, the known absolute configuration is indicated as R or S stereochemistry, as appropriate, and by use of a wedge-shaped bond (e.g. [symbol] ). When the stereochemistry at the chiral carbon atom in moiety U is unknown, the unknown absolute configuration is indicated as “rel-R” or “rel-S”, and by use of a rectangular bond (e.g. [symbol] ).

Temperatures are given in degrees Celsius (° C.). The reactants used in the examples below may be obtained from commercial sources or they may be prepared from commercially available starting materials as described herein or by methods known in the art. All the compounds of the invention are synthesized according to the Examples described herein. The progress of the reactions described herein were followed as appropriate by e.g. LC, GC or TLC, and as the skilled person will readily realize, reaction times and temperatures may be adjusted accordingly.

### NMR Spectroscopy:

Solids/oils were solubilized in DMSO-d₆ or MeOH-d₄, vortexed vigorously until the solution was clear and transferred to an NMR tube for data acquisition.

Liquid-state NMR experiments were recorded using:
600 MHz (14.1 Tesla) Bruker Avance III NMR spectrometer (600 MHz for ¹H, 151 MHz for ¹³C) using a triple-resonance ¹H, ¹⁵N, ¹³C CP-TCI 5 mm cryoprobe (Bruker Biospin, Germany)
500 MHz (11.75 Tesla) Bruker Avance I NMR spectrometer (500 MHz for ¹H, 125 MHz for ¹³C) using a Dual Resonance BBI 5 mm probe (Bruker Biospin, Germany)
400 MHz (9.4 Tesla) Bruker Avance NEO NMR spectrometer (400 MHz for ¹H, 100 MHz for ¹³C) using a SEI 5 mm probe (Bruker Biospin, Germany)
400 MHz (9.4 Tesla) Bruker Avance NEO NMR spectrometer using a PI HR-BB0400S1-BBF/H/D-5.0-Z SP probe (Bruker Biospin, Germany)
300 MHz Bruker AVANCE III HD NMR spectrometer using a PA BBO 300S1 BBF—H-D-05 Z probe (Bruker Biospin, Germany)
300 MHz Bruker Avance NEO NMR spectrometer using a PA BBO BBF—H-D-05 Z (Bruker Biospin, Germany)

All the experiments used for the resonance assignment procedure and the elucidation of the products structure (1D ¹H, 2D ¹H-¹H-COSY, 2D ¹H-¹H-ROESY, 2D ¹H-¹³C-HSQC, 2D ¹H-¹³C-HMBC) were recorded at 300 K. ¹H chemical shifts are reported in δ ppm as s (singlet), d (doublet), t (triplet), q (quartet), dd (double doublet), m (multiplet) or br s (broad singlet).

### UPLC-MS Chromatography:

UPLC-MS chromatography analysis were recorded using the following apparatus using:
Waters UPLC: Acquity, UV: Acquity PDA, MS: Qda, ELSD
Waters UPLC: Acquity, UV: Acquity TUV, MS: Qda
Waters UPLC: Acquity, UV: Acquity PDA, MS: Qda

The apparatus was tested using a CSH C18 Waters column (50×2.1 mm), 1.7 μm. It used a combination of the following eluents: H₂O+0.05% TFA (v/v) (solvent A) and MeCN+0.035% TFA (v/v) (solvent B) and a positive electrospray ES+ as ionization mode. The UV detection was set at 220 and 254 nm. The methods used were the following:
Polar method (1.7 min run): gradient t=0 2% B, t=0.5 min 2% B, t=1.5 min 98% B, t=1.52 min 2% B, t=1.7 min 2% B
Polar method (3.5 min run): gradient t=0 2% B, t=1.0 min 2% B, t=2.4 min 98% B, t=3.00 min 98% B, t=3.03 min 2% B, t=3.5 min 2% B
Normal method (1.7 min run): gradient t=0 2% B, t=1.0 min 98% B, t=1.5 min 98% B, t=1.52 min 2% B, t=1.7 min 2% B
Normal method (3.5 min run): gradient t=0 2% B, t=2.4 min 98% B, t=3.0 min 98% B, t=3.03 min 2% B, t=3.5 min 2% B Where it is observed mass spectra analysis is reported as [M+H]⁺ for the molecular ion; Someone skilled in the art will also appreciate that isotope patterns may be reported where evident, for example [M+H+2]⁺ represents the higher mass isotopic peak of Br if it is present in the molecule; Additionally, some fragmentation ions may be included in the analysis, for example [M+H−tBu]⁺, [M+H−100]⁺, [M+H-Boc]⁺ and [M−Cl+MeOH]⁺.

### Preparative HPLC

Where Example purification has been performed by preparative HPLC (chiral or achiral) the conditions are included in the reaction Step where the purification was performed or one of the general Methods below was used:

### Method A:

Gilson system used for preparative HPLC purification purposes. The system is equipped with a 333 pump A and a 334 pump B, Gilson Verity 1741 detector and GX-271 liquid handler fraction collector. The reverse phase purification was carried out by using a preparative column X-BRIDGE C18 (250×30 mm), 5 μM. Gradient elution was done using 10 mmol ammonium bicarbonate in water (as Phase A) and 100% Acetonitrile (as Phase B) with a gradient Program (B %): 10% B at 0 min hold until 3 min, 35% B at 10.0 min, 65% B at 35 min, 99% B at 35.1 min hold till 40.0 min, 10% B at 40.1 min hold until 45 min. Flow rate: 25 mL/min.

### Method B:

Gilson system used for preparative HPLC purification purposes. The system is equipped with a 333 pump A and a 334 pump B, Gilson Verity 1741 detector and GX-271 liquid handler Fraction collector. The reverse phase purification is carried out by using preparative column YMC-ACTUS C18 (250*20 mm) 5 μM. Gradient elution is done with 0.1% Formic acid in water (as Phase A) and 100% Acetonitrile (Phase B) with a gradient Program (B %): 5% B at 0 min hold till 5 min, 15% B at 200.0 min, 35% B at 35 min, 99% B at 35.1 min hold till 40.0 min, 5% B at 40.1 min hold till 45 min. Flow rate: 18 mL/min.

### Abbreviations:

In addition to the definitions above, the following abbreviations are used in the Example experimental procedures below. If an abbreviation used herein is not defined, it has its generally accepted meaning:

| | |
|---|---|
| Ac | Acetyl |
| AcOH | Acetic acid |
| AIBN | 2,2′-Azobis(2-methylpropionitrile) |
| BINAP | 2,2′-Bis(diphenylphosphino)-1,1′-binaphthalene |
| Boc | tert-butyloxycarbonyl |
| Boc₂O | Di-tertbutyl decarbonate |
| BrettPhos | 2-(Dicyclohexylphosphino)3,6-dimethoxy-2′,4′,6′-triisopropyl-1,1′- |
| | biphenyl |
| BrettPhos Pd G3 | [(2-Di-cyclohexylphosphino-3,6-dimethoxy-2′,4′,6′-triisopropyl-1,1′- |
| | biphenyl)-2-(2′-amino-1,1′-biphenyl)]palladium(II) methanesulfonate |
| | methanesulfonate |
| CataCXium A | Di(1-adamantyl)-n-butylphosphine |
| CBr₄ | tetrabromomethane |
| Cs₂CO₃ | Cesium carbonate |
| CuI | Copper (I) iodide |
| CyJohnPhos | 2-(Dicyclohexylphosphino)biphenyl |
| DavePhos | 2-Dicyclohexylphosphino-2′-(N,N-dimethylamino)biphenyl |
| DBU | 1,8-Diazabicyclo[5.4.0]undec-7-ene |
| DCE | 1,2-dichloroethane |
| DCM | Dichloromethane |
| DHP | 3,4-Dihydro-2H-pyran |
| DIAD | Diisopropyl azodicarboxylate |
| DIPEA | N,N-Diisopropylethylamine |
| Dioxane | 1,4-dioxane |
| DMAP | 4-Dimethylaminopyridine |
| DMEDA | N,N′-dimethylethylenediamine |
| DMF | Dimethylformamide |
| DMP | 1,1,1-Tris(acetyloxy)-1,1-dihydro-1,2-benziodoxol-3-(1H)-one (Dess- |
| | Martin Periodinane) |
| DMSO | Dimethylsulfoxide |
| DMSO-d₆ | Hexadeuterodimethylsulfoxide |
| ee | Enantiomeric excess |
| Et | Ethyl |
| EtOAc | Ethyl acetate |
| Et₃N | Triethylamine |
| EtOH | Ethanol |
| Et₂O | Diethylether |
| EPhos Pd G4 | Methanesulfonato{Dicyclohexyl[3-(1-methylethoxy)-2′,4′,6′-tris(1- |
| | methylethyl)[1,1′-biphenyl]-2-yl]phosphine}(2′-methylamino-1,1′- |
| | biphenyl-2-yl)palladium(II) |
| FeCl₃ | Iron(III) chloride |
| GC | Gas chromatography |
| GPhos | (3-(tert-Butoxy)-2′,6′-diisopropyl-6-methoxy-[1,1′-biphenyl]-2- |
| | yl)dicyclohexylphosphane |
| Gphos Pd G₆ TES | GPhos OAc precatalyst TES |
| h | hour |
| H₂O | water |
| HATU | 1-[Bis(dimethylamino)methylene]-1H-1,2,3-triazolo[4,5-b]pyridinium |
| | 3-oxid hexafluorophosphate |
| HCl | Hydrochloric acid |
| Het | Heteroaromatic |
| HPLC | High performance liquid chromatography |
| K₂CO₃ | Potassium carbonate |
| KI | Potassium iodide |
| LC | Liquid chromatography |
| LDA | Lithium diisopropylamide |
| LiBH₄ | Lithium borohydride |
| LiHMDS | Lithium bis(trimethylsilyl)amide |
| LiOH | Lithium hydroxide |
| min | Minutes |
| Me | Methyl |
| MeCN | Acetonitrile |
| MeOH | Methanol |
| MeTHF | 2-Methyltetrahydrofuran |
| MgSO₄ | Magnesium sulfate |
| MS | Mass spectrometry |
| MsCl | Methanesulfonyl chloride |
| MsOH | Methanesulfonic acid |
| MTBE | Methyl tert-butyl ether |
| MW | Microwave |
| N₂ | Nitrogen |
| NaH | Sodium hydride |
| NaOCN | Sodium cyanate |
| NaOH | Sodium hydroxide |
| Na₂SO₄ | Sodium sulfate |
| NaBH₃CN | Sodium cyanoborohydride |
| NBS | N-bromosuccinimide |
| NCS | N-chlorosuccinimide |
| NH₄Cl | Ammonium chloride |
| NIS | N-iodosuccinimide |
| NMP | N-methyl-2-pyrrolidone |
| Pd/C | Palladium on carbon |
| Pd(dppf)Cl₂ | [1,1′-Bis(diphenylphosphino)ferrocene]dichloropalladium(II) |
| Pd-PEPPSI-IHept-Cl | Palladium) 1,3-bis[2,6-bis(heptan-4-yl)phenyl]-4,5-dichloro-1,2- |
| | didehydro-1λ⁵-imidazole 3-chloropyridine dichloride |
| Pd(PPh₃)₂Cl₂ | Bis(triphenylphosphine)palladium(II) dichloride |
| PE | Petroleum ether |
| PPh₃ | Triphenylphosphine |
| PMB-Cl | 4-methoxybenzyl chloride |
| rt | room temperature (18 to 22° C.) |
| RuPhos | 2-Dicyclohexylphosphino-2′,6′-diisopropoxybiphenyl |
| RuPhos Pd G3 | (2-Dicyclohexylphosphino-2′,6′-diisopropoxy-1,1′-biphenyl)[2-(2′- |
| | amino-1,1′-biphenyl)]palladium(II) methanesulfonate |
| Selectfluor | 1-(Chloromethyl)-4-fluoro-1,4-diazabicyclo[2.2.2]octane-1,4-diium |
| | ditetrafluoroborate |
| SEM-Cl | 2-(Trimethylsilyl)ethoxymethyl chloride |
| TBS-Cl | tert-Butyldimethylsilyl chloride |
| tBuOK | Potassium tert-butoxide |
| TFA | Trifluoroacetic acid |
| TfOH | Trifluoromethanesulfonic acid |
| TLC | Thin layer chromatography |
| THF | Tetrahydrofuran |
| TsCl | p-Toluenesulfonyl chloride |
| TsOH | p-Toluenesulfonic acid monohydrate |
| UPLC | Ultra Performance Liquid Chromatography |
| Xantphos | 4,5-Bis(diphenylphosphino)-9,9-dimethylxanthene |

### Example 1 was Synthesised Following Scheme 1

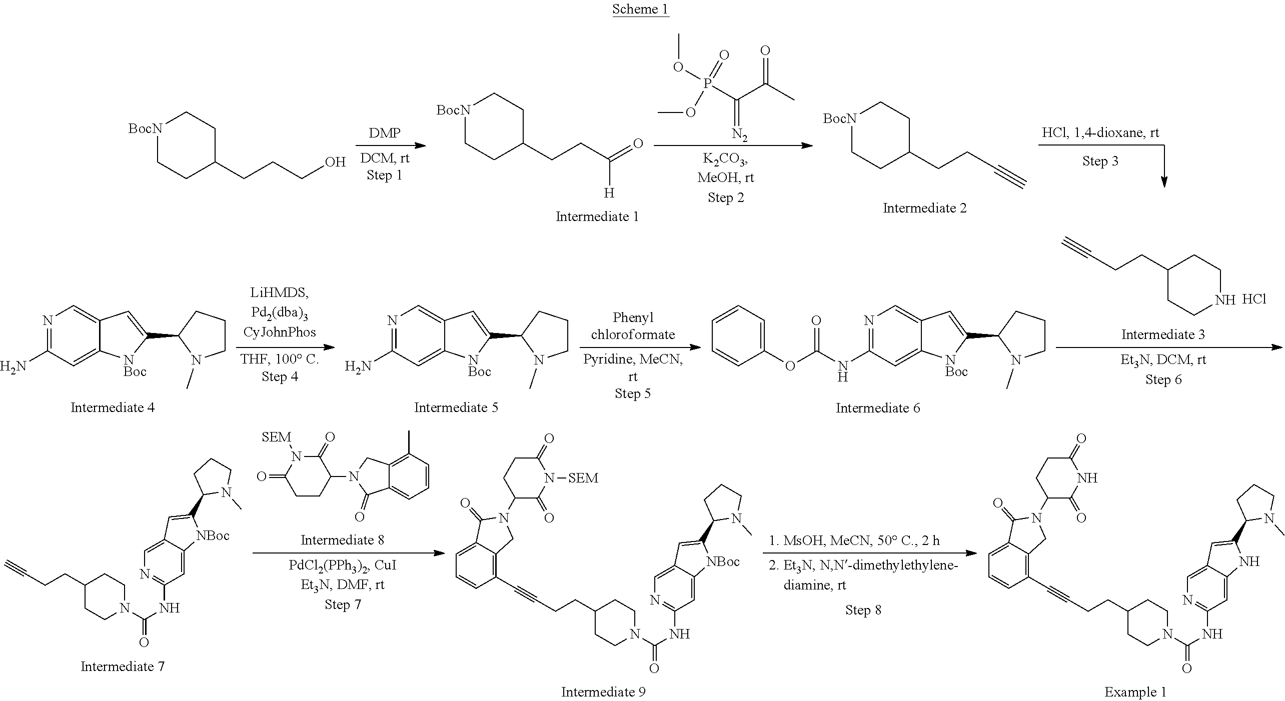

### Step 1

### Intermediate 1: tert-butyl 4-(3-oxopropyl)piperidine-1-carboxylate

To a solution of tert-butyl 4-(3-hydroxypropyl)piperidine-1-carboxylate (CAS No: 156185-63-6, 5.00 g, 20.55 mmol) in dichloromethane (100 mL) at 0° C. was added Dess-Martin periodinane (13.48 g, 30.83 mmol) and the reaction stirred at room temperature for 6 h. The reaction mixture was diluted with a mixture of sodium bicarbonate (200 mL) and sodium thiosulphate (200 mL) and extracted with dichloromethane (3×150 mL). The combined organic phases were dried over anhydrous Na₂SO₄ and concentrated in vacuo. The crude material was purified by combi-flash column chromatography by eluting with 20% ethyl acetate in heptane to afford tert-butyl 4-(3-oxopropyl)piperidine-1-carboxylate (Intermediate 1, 3.60 g, 73%) as a yellow liquid. ¹H NMR (400 MHz, DMSO-d₆) δ ppm 0.88-1.00 (m, 2H), 1.38 (s, 9H), 1.41-1.50 (m, 3H), 1.58-1.64 (m, 2H), 2.42-2.47 (m, 2H), 2.60-2.70 (m, 2H), 3.88-3.94 (m, 2H), 9.67 (s, 1H). Mass spec: m/z: Mass spec: m/z: 240.0 [M−H]⁻.

### Step 2

### Intermediate 2: tert-butyl 4-(but-3-yn-1-yl) piperidine-1-carboxylate

To a solution of tert-butyl 4-(3-oxopropyl) piperidine-1-carboxylate (Intermediate 1, 3.60 g, 15 mmol) in methanol (50 mL) was added potassium carbonate (4.10 g, 30 mmol) followed by dimethyl (1-diazo-2-oxopropyl)phosphonate (3.20 g, 16 mmol) and the reaction mixture was stirred at room temperature for 3 h. The reaction mixture was diluted with water (200 mL) and extracted with ethyl acetate (3×200 mL). The combined organic phases were dried over anhydrous Na₂SO₄, filtered, and concentrated in vacuo. The crude material was purified by combi-flash column chromatography by eluting with 20% ethyl acetate in heptane to afford tert-butyl 4-(but-3-yn-1-yl)piperidine-1-carboxylate (Intermediate 2, 2.70 g, 69% d) as a yellow liquid. ¹H NMR (400 MHz, DMSO-d₆) δ ppm 0.89-1.00 (m, 2H), 1.38 (s, 9H), 1.41-1.55 (m, 3H), 1.58-1.64 (m, 2H), 2.11-2.23 (m, 2H), 2.58-2.71 (m, 2H), 2.73 (s, 1H), 3.86-3.94 (m, 2H).

### Step 3

### Intermediate 3: 4-(but-3-yn-1-yl)piperidine hydrochloride

To a solution of tert-butyl 4-(but-3-yn-1-yl) piperidine-1-carboxylate (Intermediate 2, 2.70 g, 11 mmol) in 1,4-dioxane (30 mL) was added 4M hydrochloric acid in 1,4-dioxane (30 mL) at 0° C. and the reaction mixture was stirred at room temperature for 4 h. The reaction mixture was concentrated in vacuo to afford 4-(but-3-yn-1-yl)piperidine hydrochloride (Intermediate 3, 1.60 g) as a white solid, which was used for the next step without further purification. ¹H NMR (400 MHz, DMSO-d₆) δ ppm 1.21-1.35 (m, 2H), 1.38-1.44 (m, 2H), 1.58-1.64 (m, 1H), 1.76-1.82 (m, 2H), 2.12-2.26 (m, 2H), 2.73-2.88 (m, 3H), 3.17-3.27 (m, 2H), 8.72 (br s, 2H).

### Step 4

### Intermediate 5: tert-butyl (R)-6-amino-2-(1-methylpyrrolidin-2-yl)-1H-pyrrolo[3,2-c]pyridine-1-carboxylate

To a solution of tert-butyl (R)-6-bromo-2-(1-methylpyrrolidin-2-yl)-1H-pyrrolo[3,2-c]pyridine-1-carboxylate (Intermediate 4, 1.00 g, 2.63 mmol) in tetrahydrofuran (20 mL) was added 2-(dicyclohexylphosphino)biphenyl (CyJohnPhos, 0.19 g, 0.52 mmol) followed by Pd₂(dba)₃ (0.25 g, 0.26 mmol) at room temperature and the reaction mixture was purged with argon for 15 min. LiHMDS (1M in THF, 7.89 mL, 7.89 mmol) was then added and the reaction mixture was further purged with argon for 10 min and then stirred at 100° C. for 2 h. The reaction mixture was quenched with saturated aqueous NH₄Cl solution (50 mL) and extracted with ethyl acetate (2×50 mL). The combined organic phases were dried over anhydrous Na₂SO₄, filtered, and concentrated in vacuo. The crude material was purified by combi-flash chromatography by eluting with 90% ethyl acetate in heptane to afford tert-butyl (R)-6-amino-2-(1-methylpyrrolidin-2-yl)-1H-pyrrolo[3,2-c]pyridine-1-carboxylate (Intermediate 5, 0.80 g, 96%) as a yellow solid. ¹H NMR (400 MHz, DMSO-d₆) δ ppm 1.52-1.58 (m, 1H), 1.63 (s, 9H), 1.68-1.75 (m, 2H), 2.25-2.28 (m, 1H), 2.31 (s, 3H), 2.34-2.36 (m, 1H), 3.06-3.13 (m, 1H), 3.76-3.82 (m, 1H), 5.69 (br s, 2H), 6.49 (s, 1H), 7.01 (s, 1H), 8.12 (s, 1H). Mass spec: m/z: 317 [M+H]⁺.

### Step 5

### Intermediate 6: tert-butyl (R)-2-(1-methylpyrrolidin-2-yl)-6-((phenoxycarbonyl)amino)-1H-pyrrolo[3,2-c]pyridine-1-carboxylate

To a solution of tert-butyl (R)-6-amino-2-(1-methylpyrrolidin-2-yl)-1H-pyrrolo[3,2-c]pyridine-1-carboxylate (Intermediate 5, 1.00 g, 3.161 mmol) in acetonitrile (15 mL) was added pyridine (0.52 mL, 6.32 mmol) at room temperature and stirred for 15 min. Phenyl chloroformate (0.54 g, 3.48 mmol) was added and the reaction stirred at room temperature for 16 h. The reaction mixture was quenched with ice cold water (50 mL) and extracted with ethyl acetate (2×50 mL). The combined organic phases were dried over anhydrous Na₂SO₄, filtered and concentrated in vacuo to afford tert-butyl (R)-2-(1-methylpyrrolidin-2-yl)-6-((phenoxycarbonyl)amino)-1H-pyrrolo[3,2-c]pyridine-1-carboxylate (Intermediate 6, 0.65 g, 47%) as a white solid, which was used for the next step without further purification. ¹H NMR (400 MHz, DMSO-d₆) δ ppm 1.52-1.58 (m, 1H), 1.61 (s, 9H), 1.69-1.79 (m, 2H), 2.25-2.29 (m, 1H), 2.34 (s, 3H), 2.38-2.42 (m, 1H), 3.09-3.14 (m, 1H), 3.86-3.92 (m, 1H), 6.72 (s, 1H), 7.19-7.30 (m, 3H), 7.40-7.47 (m, 2H), 8.48 (s, 1H), 8.53 (s, 1H), 10.62 (s, 1H). Mass spec: m/z: 437.3 [M+H]⁺.

### Step 6

### Intermediate 7: tert-butyl (R)-6-(4-(but-3-yn-1-yl)piperidine-1-carboxamido)-2-(1-methylpyrrolidin-2-yl)-1H-pyrrolo[3,2-c]pyridine-1-carboxylate

To a solution of 4-(but-3-yn-1-yl)piperidine hydrochloride (Intermediate 3, 0.068 g, 0.39 mmol) in dichloromethane (2.0 mL) was added tert-butyl (R)-2-(1-methylpyrrolidin-2-yl)-6-((phenoxycarbonyl)amino)-1H-pyrrolo[3,2-c]pyridine-1-carboxylate (Intermediate 6, 0.17 g, 0.38 mmol) followed by triethylamine (0.28 mL, 2.0 mmol) and the reaction mixture was stirred at room temperature for 16 h. The reaction mixture was diluted with water (50 mL) and extracted with ethyl acetate (3×50 mL). The combined organic phases were dried over anhydrous Na₂SO₄, filtered, and concentrated in vacuo. The crude material was purified by combi-flash column chromatography by eluting with 80% ethyl acetate in heptane to afford tert-butyl (R)-6-(4-(but-3-yn-1-yl) piperidine-1-carboxamido)-2-(1-methylpyrrolidin-2-yl)-1H-pyrrolo[3,2-c]pyridine-1-carboxylate (Intermediate 7, 0.09 g, 48%) as a yellow solid. ¹H NMR (400 MHz, DMSO-d₆) δ ppm 0.98-1.11 (m, 2H), 1.36-1.44 (m, 2H), 1.56-1.60 (m, 2H), 1.65 (s, 9H), 1.68-1.78 (m, 4H), 2.18-2.22 (m, 2H), 2.27-2.31 (m, 1H), 2.33 (s, 3H), 2.36-2.38 (m, 1H), 2.71-2.80 (m, 3H), 3.10-3.16 (m, 1H), 3.84-3.90 (m, 1H), 4.14-4.18 (m, 2H), 6.66 (s, 1H), 8.42 (s, 1H), 8.50 (s, 1H), 8.90 (s, 1H). Mass spec: m/z: 480.1 [M+H]⁺.

### Step 7

### Intermediate 9: tert-butyl 6-(4-(4-(2-(2,6-dioxo-1-((2-(trimethylsilyl)ethoxy)methyl)piperidin-3-yl)-1-oxoisoindolin-4-yl)but-3-yn-1-yl)piperidine-1-carboxamido)-2-((R)-1-methylpyrrolidin-2-yl)-1H-pyrrolo[3,2-c]pyridine-1-carboxylate

To a solution of tert-butyl (R)-6-(4-(but-3-yn-1-yl) piperidine-1-carboxamido)-2-(1-methylpyrrolidin-2-yl)-1H-pyrrolo[3,2-c]pyridine-1-carboxylate (Intermediate 7, 0.33 g, 0.69 mmol) in 1,4-dioxane (7.0 mL) was added 3-(4-iodo-1-oxoisoindolin-2-yl)-1-((2-(trimethylsilyl)ethoxy)methyl)piperidine-2,6-dione (Intermediate 8, 0.34 g, 0.68 mmol) followed by triethylamine (3.50 mL, 25.0 mmol). The reaction mixture was purged with argon for 15 min then PdCl₂(PPh₃)₂ (0.05 g, 0.070 mmol) and copper(I) iodide (0.014 g, 0.072 mmol) were added. The reaction mixture was further purged with argon for 10 min and then stirred at room temperature for 2 h. The reaction mixture was diluted with water (100 mL) and extracted with ethyl acetate (2×100 mL). The combined organic phases were dried over anhydrous Na₂SO₄, filtered, and concentrated in vacuo. The crude material was purified by combi-flash column chromatography by eluting with 80% ethyl acetate in heptane to afford tert-butyl 6-(4-(4-(2-(2,6-dioxo-1-((2-(trimethylsilyl)ethoxy)methyl)piperidin-3-yl)-1-oxoisoindolin-4-yl)but-3-yn-1-yl)piperidine-1-carboxamido)-2-((R)-1-methylpyrrolidin-2-yl)-1H-pyrrolo[3,2-c]pyridine-1-carboxylate (Intermediate 9, 0.29 g, 49%) as a yellow solid. ¹H NMR (400 MHz, DMSO-d₆) δ ppm −0.07 (s, 9H), 0.76-0.82 (m, 2H), 1.05-1.10 (m, 3H), 1.46-1.56 (m, 3H), 1.62 (s, 9H), 1.63-1.65 (m, 1H), 1.67-1.78 (m, 4H), 2.00-2.09 (m, 1H), 2.26-2.28 (m, 2H), 2.31 (s, 3H), 2.36-2.38 (m, 1H), 2.69-2.83 (m, 3H), 2.97-3.12 (m, 2H), 3.33-3.40 (m, 1H), 3.46-3.52 (m, 2H), 3.82-3.86 (m, 1H), 4.14-4.18 (m, 2H), 4.23-4.27 (m, 1H), 4.43-4.48 (m, 1H), 4.95-5.08 (m, 2H), 5.23-5.28 (m, 1H), 6.63 (s, 1H), 7.48-7.52 (m, 1H), 7.63 (d, J=7.40 Hz, 1H), 7.69 (d, J=7.40 Hz, 1H), 8.39 (s, 1H), 8.48 (s, 1H), 8.88 (br s, 1H). Mass spec: m/z: 852.0 [M+H]⁺.

### Step 8

### Example 1: 4-(4-(2-(2,6-dioxopiperidin-3-yl)-1-oxoisoindolin-4-yl)but-3-yn-1-yl)-N-(2-((R)-1-methylpyrrolidin-2-yl)-1H-pyrrolo[3,2-c]pyridin-6-yl)piperidine-1-carboxamide

To a solution of tert-butyl 6-(4-(4-(2-(2,6-dioxo-1-((2-(trimethylsilyl)ethoxy)methyl)piperidin-3-yl)-1-oxoisoindolin-4-yl)but-3-yn-1-yl)piperidine-1-carboxamido)-2-((R)-1-methylpyrrolidin-2-yl)-1H-pyrrolo[3,2-c]pyridine-1-carboxylate (Intermediate 9, 0.60 g, 0.70 mmol) in acetonitrile (10 mL) was added methanesulfonic acid (0.47 mL, 7.1 mmol) at room temperature and the reaction mixture was stirred at 50° C. for 2 h. N,N′-dimethylethylenediamine (0.43 mL, 3.6 mmol) and triethylamine (1.98 mL, 14.1 mmol) were added and the reaction stirred at room temperature for 3 h. The reaction mixture was concentrated in vacuo to obtain crude compound. Water (50 mL) was added resulting in a precipitate which was collected by filtration and dried in vacuo. The crude material was purified by preparative HPLC (Method A) to afford 4-(4-(2-(2,6-dioxopiperidin-3-yl)-1-oxoisoindolin-4-yl) but-3-yn-1-yl)-N-(2-((R)-1-methylpyrrolidin-2-yl)-1H-pyrrolo[3,2-c]pyridin-6-yl) piperidine-1-carboxamide (Example 1, 0.15 g, 35%) as an off white solid. ¹H NMR (400 MHz, DMSO-d₆) δ ppm 1.04-1.17 (m, 2H), 1.53-1.58 (m, 2H), 1.60-1.68 (m, 1H), 1.70-1.78 (m, 2H), 1.80-1.95 (m, 2H), 1.99-2.06 (m, 1H), 2.08-2.13 (m, 1H), 2.14 (s, 3H), 2.21-2.28 (m, 1H), 2.40-2.46 (m, 2H), 2.53-2.64 (m, 3H), 2.72-2.80 (m, 2H), 2.87-2.96 (m, 1H), 3.10-3.16 (m, 1H), 3.24-3.28 (m, 1H), 4.13-4.23 (m, 2H), 4.29-4.36 (m, 1H), 4.44-4.50 (m, 1H), 5.09-5.18 (m, 1H), 6.30 (s, 1H), 7.49-7.55 (m, 1H), 7.63-7.66 (m, 1H), 7.71-7.46 (m, 1H), 7.74 (s, 1H), 8.35 (s, 1H), 8.60 (s, 1H), 11.05 (s, 1H), 11.13 (s, 1H). Mass spec: m/z: 622.1 [M+H]⁺.

### Intermediate 4 was Synthesised Following Scheme 2

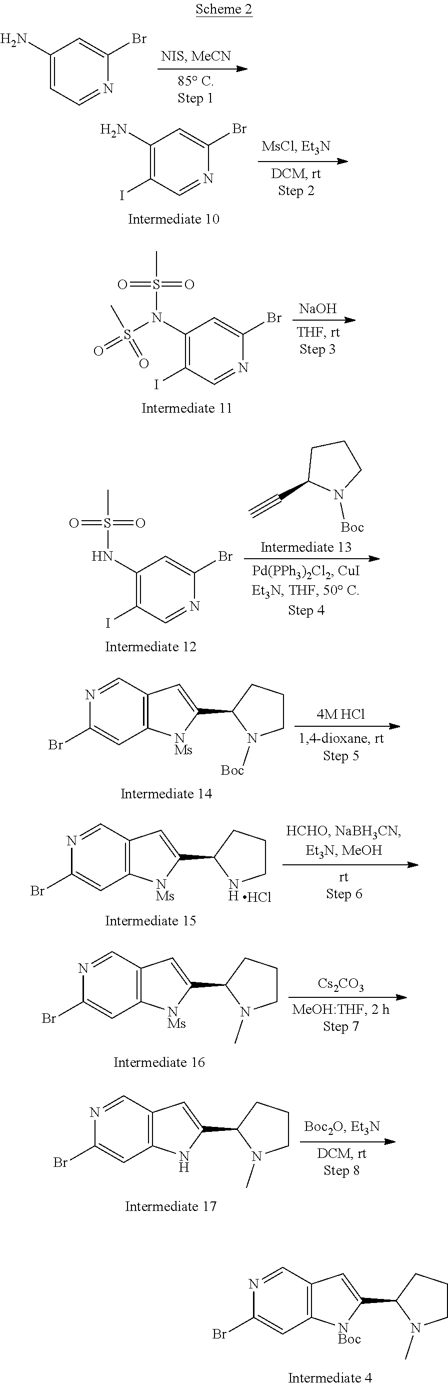

### Step 1

### Intermediate 10: 2-bromo-5-iodopyridin-4-amine

To solution of 2-bromopyridin-4-amine (CAS No: 7598-35-8, 200 g, 1156.0 mmol) in acetonitrile (1 L) was added N-iodosuccinimide (297.23 g, 1294.7 mmol) and the reaction mixture was heated at 80° C. for 16 h. The reaction mixture was then cooled to room temperature and concentrated in vacuo. The obtained residue was diluted with aqueous saturated sodium thiosulphate solution (2 L) and extracted with ethyl acetate (3×2 L). The combined organic layers were dried over anhydrous Na₂SO₄ and concentrated in vacuo to obtain the crude compound. The crude material was purified by combi-flash chromatography, by eluting with 5% ethyl acetate in heptane, to afford 2-bromo-5-iodo-pyridin-4-amine (Intermediate 10, 115 g, 33%) as a yellow solid. ¹H NMR (400 MHz, DMSO-d₆) δ ppm 6.48 (br s, 2H), 6.77 (s, 1H), 8.16 (s, 1H). Mass spec: m/z: 300.8 [M+H]⁺.

### Step 2

### Intermediate 11: N-(2-bromo-5-iodopyridin-4-yl)-N-(methylsulfonyl)methanesulfonamide

To a cooled (0° C.) solution of 2-bromo-5-iodo-pyridin-4-amine (Intermediate 10, 100 g, 334.55 mmol) in dichloromethane (2.50 g, 29 mmol) was added triethylamine (234 mL, 1673 mmol) followed by methanesulfonyl chloride (106 mL, 1338.2 mmol) in dichloromethane (300 mL). After the addition was complete, the reaction mixture was stirred at room temperature for 3 h. The reaction mixture was quenched with aqueous saturated NaHCO₃ solution (2 L) and extracted with dichloromethane (2×2 L). The combined organic layers were dried over anhydrous Na₂SO₄ and concentrated in vacuo to afford N-(2-bromo-5-iodopyridin-4-yl)-N-(methylsulfonyl)methanesulfonamide (Intermediate 11, 150 g, 98%) as a yellow liquid, which was used for next step without further purification. ¹H NMR (400 MHz, DMSO-d₆) δ 3.65 (s, 6H), 8.10 (s, 1H), 8.92 (s, 1H). Mass spec: m/z: 456 [M+H]⁺.

### Step 3

### Intermediate 12: N-(2-bromo-5-iodopyridin-4-yl)methanesulfonamide

To a solution of N-(2-bromo-5-iodopyridin-4-yl)-N-(methylsulfonyl)methanesulfonamide (Intermediate 11, 150 g, 329.6 mmol) in tetrahydrofuran (2 L) and water (2 L) was added sodium hydroxide (79.9 g, 1978 mmol) and the reaction mixture was stirred at room temperature for 3 h. The reaction mixture was poured into water (500 mL), extracted with ethyl acetate (3×1000 mL) and acidified with citric acid solution (1 L) to pH ~4. The resultant precipitate was collected by filtration, washed with water (100 mL) and dried in vacuo to afford N-(2-bromo-5-iodopyridin-4-yl) methanesulfonamide (Intermediate 12, 75 g, 60%) as a yellow solid, which was used for next step without further purification. ¹H NMR (400 MHz, DMSO-d₆) δ ppm 3.28 (s, 3H), 7.54 (s, 1H), 8.64 (s, 1H), 9.51 (s, 1H). Mass spec: m/z: 378.7 [M+H]⁺.

### Step 4

### Intermediate 14: tert-butyl (R)-2-(6-bromo-1-(methylsulfonyl)-1H-pyrrolo[3,2-c]pyridin-2-yl)pyrrolidine-1-carboxylate

To a solution of N-(2-bromo-5-iodopyridin-4-yl)methanesulfonamide (Intermediate 12, 50.0 g, 132.63 mmol) and tert-butyl (R)-2-ethynylpyrrolidine-1-carboxylate (Intermediate 13, 25.89 g, 132.63 mmol) in THF (500 mL) was added N,N-diisopropylethylamine (138 g, 1061.0 mmol). The reaction mixture was purged with argon gas for 15 min then PdCl₂(PPh₃)₂ (9.60 g, 13.263 mmol) and copper(I) iodide (2.55 g, 13.26 mmol) were added. The reaction mixture was further purged with argon gas for 10 min and then heated at 60° C. for 3 h. The reaction mixture was diluted with water (500 mL) and extracted with ethyl acetate (3×300 mL). The combined organic layers were dried over anhydrous Na₂SO₄ and concentrated in vacuo to obtain crude compound. The crude material was purified by combi-flash chromatography, by eluting with 30% ethyl acetate in heptane, to afford tert-butyl (R)-2-(6-bromo-1-(methylsulfonyl)-1H-pyrrolo[3,2-c]pyridin-2-yl)pyrrolidine-1-carboxylate (Intermediate 14, 38.0 g, 62%) as a yellow solid. ¹H NMR (400 MHz, DMSO-d₆) δ ppm 1.40 ppm (s, 9H), 1.85-1.91 (m, 4H), 3.18-3.21 (m, 1H), 3.37-3.41 (m, 1H), 3.62 (s, 3H), 5.23-5.31 (m, 1H), 6.72 (s, 1H), 7.97 (s, 1H), 8.69 (s, 1H). Mass spec: m/z: 445.9 [M+H]⁺.

### Step 5

### Intermediate 15: (R)-6-bromo-1-(methylsulfonyl)-2-(pyrrolidin-2-yl)-1H-pyrrolo[3,2-c]pyridine hydrochloride

To a cooled (0° C.) solution of tert-butyl (R)-2-(6-bromo-1-(methylsulfonyl)-1H-pyrrolo[3,2-c]pyridin-2-yl)pyrrolidine-1-carboxylate (Intermediate 14, 200 g, 450.1 mmol) in 1,4-dioxane (1 L) was added 4M hydrochloric acid in 1,4-dioxane (2 L) and the reaction mixture was stirred at room temperature for 6 h. The reaction mixture was concentrated in vacuo to afford (R)-6-bromo-1-(methylsulfonyl)-2-(pyrrolidin-2-yl)-1H-pyrrolo[3,2-c]pyridine hydrochloride (Intermediate 15, 160 g) as a yellow solid, which was used for the next step without further purification. ¹H NMR (400 MHz, DMSO-d₆) δ ppm 1.87-2.20 (m, 2H), 2.33-2.46 (m, 2H), 3.15-3.34 (m, 2H), 3.75 (s, 3H), 5.12-5.17 (m, 1H), 7.38 (s, 1H), 7.98 (s, 1H), 8.78 (s, 1H), 9.45 (s, 2H). Mass spec: m/z: 345.8 [M+H]⁺.

### Step 6

### Intermediate 16: (R)-6-bromo-2-(1-methylpyrrolidin-2-yl)-1-(methylsulfonyl)-1H-pyrrolo [3,2-c]pyridine

To a cooled (0° C.) solution of (R)-6-bromo-1-(methylsulfonyl)-2-(pyrrolidin-2-yl)-1H-pyrrolo[3,2-c]pyridine hydrochloride (Intermediate 15, 90.0 g, 236.4 mmol) in methanol (900 mL) was added triethylamine (192 g, 1891 mmol) followed by formaldehyde (60.83 g, 709.2 mmol). The reaction mixture was stirred for 5 min and then sodium cyanoborohydride (34.96 g, 472.8 mmol) was added at 0° C. The reaction mixture was then heated at 60° C. for 16 h. The reaction mixture was diluted with water (500 mL) and extracted with dichloromethane (3×500 mL). The combined organic layers were dried over anhydrous Na₂SO₄ and concentrated in vacuo to the obtain crude compound. The crude material was purified by combi-flash chromatography by eluting with 60% ethyl acetate in heptane, to afford (R)-6-bromo-2-(1-methylpyrrolidin-2-yl)-1-(methylsulfonyl)-1H-pyrrolo[3,2-c]pyridine (Intermediate 16, 60.0 g, 71%) as a yellow solid. ¹H NMR (400 MHz, DMSO-d₆) δ ppm 1.70-1.78 (m, 3H), 2.33 (s, 3H), 2.36-2.40 (m, 2H), 3.01-3.22 (m, 1H), 3.56 (s, 3H), 3.76-3.81 (m, 1H), 6.89 (s, 1H), 7.97 (s, 1H), 8.68 (s, 1H). Mass spec: m/z: 359.8 [M+H]⁺.

### Step 7

### Intermediate 17: (R)-6-bromo-2-(1-methylpyrrolidin-2-yl)-1H-pyrrolo[3,2-c]pyridine

To a solution of (R)-6-bromo-2-(1-methylpyrrolidin-2-yl)-1-(methylsulfonyl)-1H-pyrrolo[3,2-c]pyridine (Intermediate 16, 110 g, 307.1 mmol) in methanol (600 mL) was added tetrahydrofuran (600 mL) and the mixture cooled to 0° C. Cesium carbonate (200 g, 614.2 mmol) was added and the reaction mixture was stirred at room temperature for 4 h. The reaction mixture was quenched with water (2 L) and extracted with ethyl acetate (2×1 L). The combined organic layers were dried over anhydrous Na₂SO₄ and concentrated in vacuo to afford (R)-6-bromo-2-(1-methylpyrrolidin-2-yl)-1H-pyrrolo[3,2-c]pyridine (Intermediate 17, 60.0 g, 70%) as a yellow solid, which was used for the next step without further purification. ¹H NMR (400 MHz, DMSO-d₆) δ ppm 1.75-1.91 (m, 3H), 2.14 (s, 3H), 2.21-2.29 (m, 1H), 3.10-3.15 (m, 1H), 3.33-3.36 (m, 2H), 6.45 (s, 1H), 7.43 (s, 1H), 8.48 (s, 1H), 11.57 (br s, 1H). Mass spec: m/z: 279.9 [M+H]⁺.

### Step 8

### Intermediate 4: tert-butyl (R)-6-bromo-2-(1-methylpyrrolidin-2-yl)-1H-pyrrolo[3,2-c]pyridine-1-carboxylate

To a solution of (R)-6-bromo-2-(1-methylpyrrolidin-2-yl)-1H-pyrrolo[3,2-c]pyridine (Intermediate 17, 60.0 g, 214.16 mmol) in dichloromethane (600 mL) was added triethylamine (65.3 g, 642.49 mmol) followed by di-tert-butyldicarbonate (94.42 g, 428.33 mmol) and 4-(dimethylamino)pyridine (5.28 g, 42.833 mmol) and the reaction mixture was stirred at room temperature for 3 h. The reaction mixture was diluted with water (500 mL) and extracted with diethyl ether (3×500 mL). The combined organic layers were dried over anhydrous Na₂SO₄ and concentrated in vacuo to obtain the crude compound. The crude material was purified by combi-flash chromatography, by eluting with 45% ethyl acetate in heptane, followed by SFC purification (Waters SFC Prep 150 Mgm equipped with 2489 PDA detector; Column: Chiralpak IC (30×250 mm, 5 μM); Solvent A) CO₂ 70 g/min; B) 0.1% Isopropyl amine in isopropyl alcohol and MeCN (50:50) 25 mL/min) to afford tert-butyl-(R)-6-bromo-2-(1-methylpyrrolidin-2-yl)-1H-pyrrolo[3,2-c]pyridine-1-carboxylate (Intermediate 4, 43 g, 53%) as an off white solid. Enantiomeric excess (ee) 98.4%. ¹H NMR (400 MHz, DMSO-d₆) δ ppm 1.65 (s, 9H), 1.71-1.75 (m, 3H), 2.30-2.33 (m, 1H), 2.34 (s, 3H), 2.37-2.40 (m, 1H), 3.11-3.14 (m, 1H), 3.87-3.91 (m, 1H), 6.80 (s, 1H), 8.05 (s, 1H), 8.61 (s, 1H). Mass spec: m/z: 379.9 [M+H]⁺.

### Intermediate 4 can Also be Synthesised Following Scheme 3

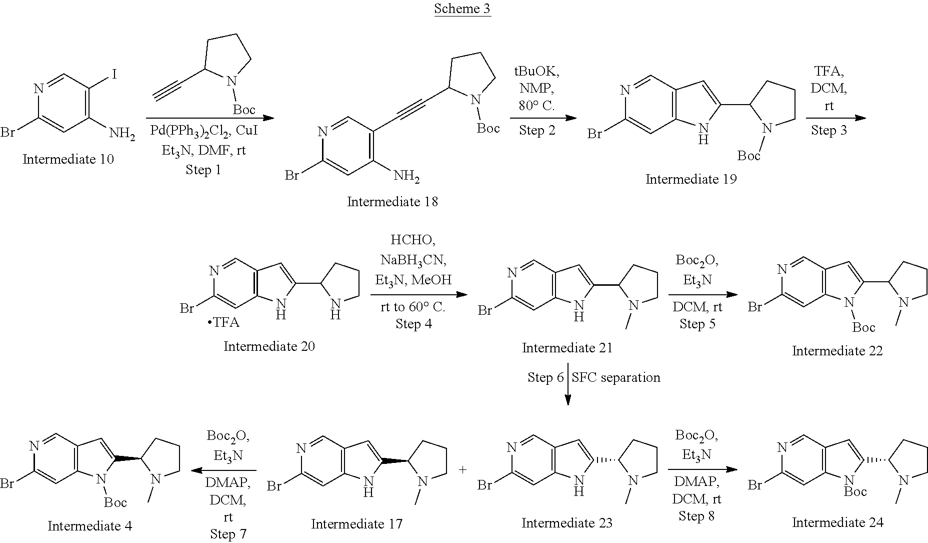

### Step 1

### Intermediate 18: rac-tert-butyl 2-[2-(4-amino-6-bromopyridin-3-yl)ethynyl]pyrrolidine-1-carboxylate

A solution of 2-bromo-5-iodopyridin-4-amine (Intermediate 10, 17 g, 54.2 mmol), rac-tert-butyl 2-ethynylpyrrolidine-1-carboxylate (CAS: 316141-37-4, 13.4 g, 65.04 mmol), Pd(PPh3)₂Cl₂ (3.81 g, 5.42 mmol), CuI (1.03 g, 5.42 mmol) and Et₃N (16.4 g, 162.6 mmol) in DMF (400 mL) was stirred for 2 h at rt under nitrogen atmosphere. The reaction mixture was quenched with water and the resulting solution was extracted three times with EtOAc. The organic layers were combined, washed with brine, dried over anhydrous Na₂SO₄, filtered, and concentrated under reduced pressure. The crude material was purified by flash chromatography on silica gel using petroleum ether/EtOAc (7/3) as eluent to afford rac-tert-butyl 2-[2-(4-amino-6-bromopyridin-3-yl)ethynyl]pyrrolidine-1-carboxylate (Intermediate 18, 18 g, 83%) as a yellow oil. Mass spec: m/z 366 [M+H]⁺.

### Step 2

### Intermediate 19: rac-tert-butyl 2-{6-bromo-1H-pyrrolo[3,2-c]pyridin-2-yl}pyrrolidine-1-carboxylate

To a solution of rac-tert-butyl 2-[2-(4-amino-6-bromopyridin-3-yl)ethynyl]pyrrolidine-1-carboxylate (Intermediate 18, 8 g, 20.8 mmol) in NMP (40 mL) was added a solution of tBuOK (65.53 mL, 65.53 mmol, 1M in THF). The reaction mixture was stirred for 4 h at 80° C. The reaction mixture was quenched with water and the resulting solution was extracted three times with EtOAc. The organic layers were combined, washed with brine, dried over anhydrous Na₂SO₄, filtered, and concentrated under reduced pressure. The crude material was purified by flash chromatography on silica gel using DCM/MeOH (95/5) as eluent to afford rac-tert-butyl 2-{6-bromo-1H-pyrrolo[3,2-c]pyridin-2-yl}pyrrolidine-1-carboxylate (Intermediate 19, 6 g, 71%) as a yellow oil. Mass spec: m/z 366 [M+H]⁺.

### Step 3

### Intermediate 20: rac-2-{6-bromo-1H-pyrrolo[3,2-c]pyridin-2-yl}pyrrolidine trifluoroacetate

To a solution of rac-tert-butyl 2-{6-bromo-1H-pyrrolo[3,2-c]pyridin-2-yl}pyrrolidine-1-carboxylate (Intermediate 19, 16 g, 41.5 mmol) in DCM (300 mL) was added TFA (30 mL).

The reaction mixture was stirred for 2 h at rt. The solution was concentrated under reduced pressure to afford rac-2-{6-bromo-1H-pyrrolo[3,2-c]pyridin-2-yl}pyrrolidine trifluoroacetate (Intermediate 20, 10 g, 77%) as a yellow oil which was used in Step 5 without further purification. Mass spec: m/z 266 [M+H]⁺.

### Step 4

### Intermediate 21: rac-6-bromo-2-(1-methylpyrrolidin-2-yl)-1H-pyrrolo[3,2-c]pyridine

To a solution of rac-6-bromo-2-(pyrrolidin-2-yl)-1H-pyrrolo[3,2-c]pyridine trifluoroacetate (Intermediate 20, 10 g, 35.8 mmol) in MeOH (300 mL) were added Et₃N (10.9 g, 108 mmol) and paraformaldehyde (5.37 g, 179 mmol). The reaction mixture was stirred for 1 h at rt. NaBH₃CN (6.77 g, 107.4 mmol) was added and the reaction mixture was stirred overnight at 60° C. The reaction mixture was quenched with water and the resulting solution was extracted three times with EtOAc. The organic layers were combined, washed with brine, dried over anhydrous Na₂SO₄, filtered, and concentrated under reduced pressure. The residue was chromatographed by reverse-phase flash chromatography (C18 aq) using MeCN/H₂O (45/55) as eluent to afford rac-6-bromo-2-(1-methylpyrrolidin-2-yl)-1H-pyrrolo[3,2-c]pyridine (Intermediate 21, 8 g, 69%) as a yellow oil. Mass spec: m/z 280 [M+H]⁺.

### Step 5

### Intermediate 22: rac-tert-butyl 6-bromo-2-(1-methylpyrrolidin-2-yl)-1H-pyrrolo[3,2-c]pyridine-1-carboxylate

To a solution of rac-6-bromo-2-(1-methylpyrrolidin-2-yl)-1H-pyrrolo[3,2-c]pyridine (Intermediate 21, 8 8 g, 25.8 mmol) in DCM (200 mL) were added Boc₂O (8.44 g, 38.7 mmol), Et₃N (7.82 g, 77.4 mmol) and DMAP (310 mg, 2.58 mmol). The reaction mixture was stirred overnight at rt and then quenched with water. The resulting solution was extracted three times with EtOAc and the organic layers were combined, washed with brine, dried over anhydrous Na₂SO₄, filtered, and concentrated under reduced pressure. The crude material was purified by flash chromatography on silica gel using EtOAc/petroleum ether (3/2) as eluent to afford rac-tert-butyl 6-bromo-2-(1-methylpyrrolidin-2-yl)-1H-pyrrolo[3,2-c]pyridine-1-carboxylate (Intermediate 22, 8.1 g, 71%) as a yellow oil. Mass spec: m/z 380 [M+H]⁺.

### Step 6

### Intermediate 17: (2R)-2-{6-bromo-1H-pyrrolo[3,2-c]pyridin-2-yl}-1-methylpyrrolidine and Intermediate 23: (2S)-2-{6-bromo-1H-pyrrolo[3,2-c]pyridin-2-yl}-1-methylpyrrolidine

rac-6-bromo-2-(1-methylpyrrolidin-2-yl)-1H-pyrrolo[3,2-c]pyridine (Intermediate 21, 9.00 g, 30.5 mmol) was separated by SFC (Column: CHIRALPAK 1H 3*25 cm, 5 m; Mobile Phase A: CO₂, Mobile Phase B: MeOH (+1%-2M NH₃ in MeOH); Flow rate: 85 mL/min; Gradient: isocratic 20% B) to afford (2R)-2-{6-bromo-1H-pyrrolo[3,2-c]pyridin-2-yl}-1-methylpyrrolidine (Intermediate 17, 1st eluting peak, 3.5 g, 37%) as a white solid and (2S)-2-{6-bromo-1H-pyrrolo[3,2-c]pyridin-2-yl}-1-methylpyrrolidine (Intermediate 23, 2nd eluting peak, 4.0 g, 40%) as a white solid.

### Intermediate 17: (2R)-2-{6-bromo-1H-pyrrolo[3,2-c]pyridin-2-yl}-1-methylpyrrolidine

1^{st} eluting peak from SFC purification. Retention time: 4.35 mins.

Enantiomeric excess (ee) 99.8%. ¹H NMR (400 MHz, DMSO-d6) δ ppm 11.56 (s, 1H), 8.49 (s, 1H), 7.43 (s, 1H), 6.45 (s, 1H), 3.34 (t, J=9.0 Hz, 1H), 3.13 (t, J=7.8 Hz, 1H), 2.24-2.30 (m, 1H), 2.15 (s, 4H), 1.87-1.90 (m, 1H), 1.75-1.84 (m, 2H).

### Intermediate 23: (2S)-2-{6-bromo-1H-pyrrolo[3,2-c]pyridin-2-yl}-1-methylpyrrolidine

2nd eluting peak from SFC purification. Retention time: 4.87 mins.

Enantiomeric excess (ee) 99.8%.

¹H NMR (400 MHz, DMSO-d6) δ ppm 11.56 (s, 1H), 8.49 (s, 1H), 7.43 (s, 1H), 6.45 (s, 1H), 3.35 (t, J=11.4 Hz, 1H), 3.14 (t, J=8.0 Hz, 1H), 2.26 (t, J=8.6 Hz, 1H), 2.15 (s, 4H), 1.88 (d, J=8.0 Hz, 1H), 1.80 (d, J=8.0 Hz, 2H).

### Step 7

### Intermediate 4: tert-butyl (R)-6-bromo-2-(1-methylpyrrolidin-2-yl)-1H-pyrrolo[3,2-c]pyridine-1-carboxylate

A 500 mL round-bottom flask was charged with (2R)-2-{6-bromo-1H-pyrrolo[3,2-c]pyridin-2-yl}-1-methylpyrrolidine (Intermediate 17, 500 mg, 1.68 mmol), DCM (10 mL), Et₃N (541 mg, 5.35 mmol), DMAP (21.8 mg, 0.178 mmol), (Boc)₂0 (778 mg, 3.57 mmol) and the reaction was stirred overnight at room temperature. The reaction was quenched with water (200 mL). The resulting solution was extracted with ethyl acetate (3×200 mL) and the organic layers were combined, washed with brine (2×300 mL), dried over anhydrous sodium sulfate, filtered, and concentrated under reduced pressure. The residue was chromatographed on a silica gel column, eluting with EtOAc/Petroleum ether (35/65) to afford tert-butyl (R)-6-bromo-2-(1-methylpyrrolidin-2-yl)-1H-pyrrolo[3,2-c]pyridine-1-carboxylate (Intermediate 4, 600 mg, 92%) as an off-white solid. ¹H NMR (400 MHz, DMSO-d6) δ ppm 1.65 (s, 9H), 1.71-1.75 (m, 3H), 2.30-2.33 (m, 1H), 2.34 (s, 3H), 2.37-2.40 (m, 1H), 3.11-3.14 (m, 1H), 3.87-3.91 (m, 1H), 6.80 (s, 1H), 8.05 (s, 1H), 8.61 (s, 1H). Mass spec: m/z: 379.9 [M+H]+. Mass spec: m/z: 380.0 [M+H]⁺.

### Step 8

### Intermediate 24: tert-butyl (S)-6-bromo-2-(1-methylpyrrolidin-2-yl)-1H-pyrrolo[3,2-c]pyridine-1-carboxylate

Intermediate 24 was prepared in an analogous manner to Intermediate 4 following Step 7 of Scheme 3 and exhibited the following data: ¹H NMR (400 MHz, DMSO-d6) δ ppm 1.65 (s, 9H), 1.71-1.75 (m, 3H), 2.30-2.33 (m, 1H), 2.34 (s, 3H), 2.37-2.40 (m, 1H), 3.11-3.14 (m, 1H), 3.87-3.91 (m, 1H), 6.80 (s, 1H), 8.05 (s, 1H), 8.61 (s, 1H). Mass spec: m/z: 379.9 [M+H]+. Mass spec: m/z: 380.0 [M+H]⁺.

### Intermediate 13: tert-butyl (R)-2-ethynylpyrrolidine-1-carboxylate

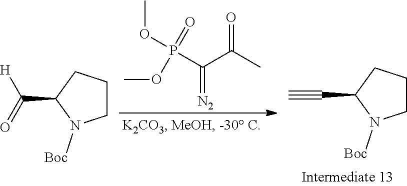

To a cooled (−30° C.) solution of tert-butyl-(R)-2-formylpyrrolidine-1-carboxylate (CAS No: 73365-02-3, 50.0 g, 250.94 mmol) in methanol (500 mL) was added potassium carbonate (51.94 g, 376.41 mmol). Dimethyl(1-diazo-2-oxopropyl)phosphonate (CAS No: 90965-06-3, 57.85 g, 301.13 mmol) was then added dropwise over 20 min and the reaction mixture was stirred at −30° C. for 3 h. The reaction mixture was diluted with water (1000 mL) and extracted with diethyl ether (3×500 mL). The combined organic layers were dried over anhydrous Na₂SO₄ and concentrated in vacuo to obtain the crude compound. The crude material was purified by combi-flash chromatography, by eluting with 10% ethyl acetate in heptane, to afford tert-butyl (R)-2-ethynylpyrrolidine-1-carboxylate (Intermediate 13, 35.0 g, 71%) as a yellow liquid. ¹H NMR (400 MHz, DMSO-d₆) δ ppm 1.40 (s, 9H), 1.81-1.89 (m, 3H), 1.99-2.12 (m, 1H), 3.08-3.25 (m, 2H), 3.26-3.35 (m, 1H), 4.32-4.38 (m, 1H).

### Intermediate 8 was Synthesised Following Scheme 4

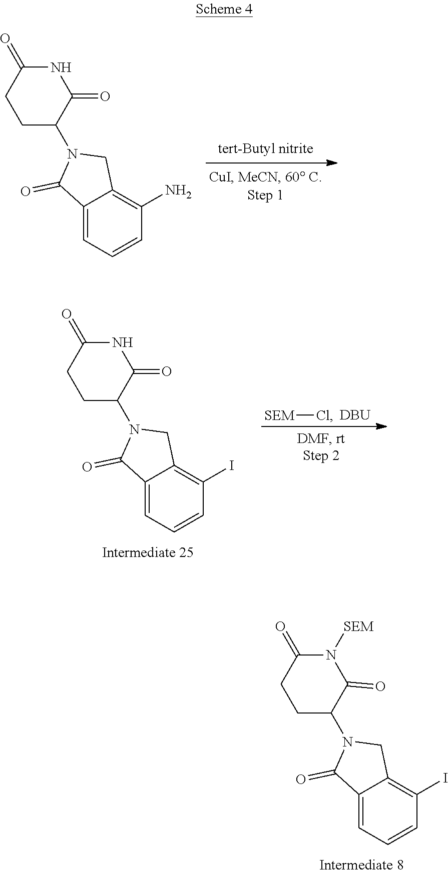

### Step 1

### Intermediate 25: 3-(4-iodo-1-oxoisoindolin-2-yl)piperidine-2,6-dione

To a solution of 3-(4-amino-1-oxoisoindolin-2-yl)piperidine-2,6-dione (CAS No 191732-72-6, 25.0 g, 96.41 mmol) in acetonitrile (300 mL) was added copper(I) iodide (28.10 g, 144.6 mmol). The reaction mixture was purged with argon gas for 20 min then tert-butyl nitrite (16.57 g, 144.6 mmol) was added and the reaction mixture was heated at 60° C. for 12 h. The reaction mixture was poured then into water (300 mL), a solid precipitate formed, which was filtered and dried under vacuum to afford 3-(4-iodo-1-oxoisoindolin-2-yl) piperidine-2,6-dione (Intermediate 25, 45.2 g) as an off white solid, which was used for the next step without further purification. ¹H NMR (400 MHz, DMSO-d6) δ ppm 1.98-2.04 (m, 2H), 2.82-2.98 (m, 2H), 4.11-4.32 (m, 2H), 5.13 (m, 1H), 7.31-7.42 (m, 1H), 7.71-7.80 (m, 1H), 7.98-8.06 (m, 1H), 10.99 (br s, 1H). Mass spec: m/z [M+H]⁺ 371.0.

### Step 2

### Intermediate 8: 3-(4-iodo-1-oxoisoindolin-2-yl)-1-((2-(trimethylsilyl) ethoxy)methyl) piperidine-2,6-dione

To solution of 3-(4-iodo-1-oxoisoindolin-2-yl)piperidine-2,6-dione (Intermediate 25, 30.0 g, 81.06 mmol) in dimethylformamide (250 mL) was added DBU (44.07 g, 283.7 mmol) at 0° C., then 2-(trimethylsilyl)ethoxymethyl chloride (52 mL, 283.7 mmol) was added and the reaction mixture was stirred at room temperature for 16 h under a nitrogen atmosphere. The reaction mixture was quenched with water (700 mL), diluted with ethyl acetate (500 mL) and filtered through a celite bed. The organic layer was separated, washed with water (500 mL), dried over anhydrous Na₂SO₄ and concentrated in vacuo. The crude material was purified by combi-flash chromatography, eluting with 50% ethyl acetate in heptane to afford 3-(4-iodo-1-oxoisoindolin-2-yl)-1-((2-(trimethylsilyl) ethoxy)methyl) piperidine-2,6-dione (Intermediate 8, 8.56 g, 21%) as an off-white solid. ¹H NMR (400 MHz, DMSO-d6) δ ppm −0.02 (s, 9H), 0.78-0.90 (m, 2H), 2.03-2.11 (m, 1H), 2.76-2.82 (m, 1H), 3.02-3.12 (m, 1H), 3.48-3.60 (m, 3H), 4.08-4.13 (m, 1H), 4.28-4.33 (m, 1H), 5.02-5.10 (m, 2H), 5.26-5.31 (m, 1H), 7.31-7.37 (m, 1H), 7.79 (d, J=7.46 Hz, 1H), 8.05 (d, J=7.46 Hz, 1H). Mass spec: m/z [M−H]⁻498.8.

### Example 2 was Synthesised Following Scheme 5

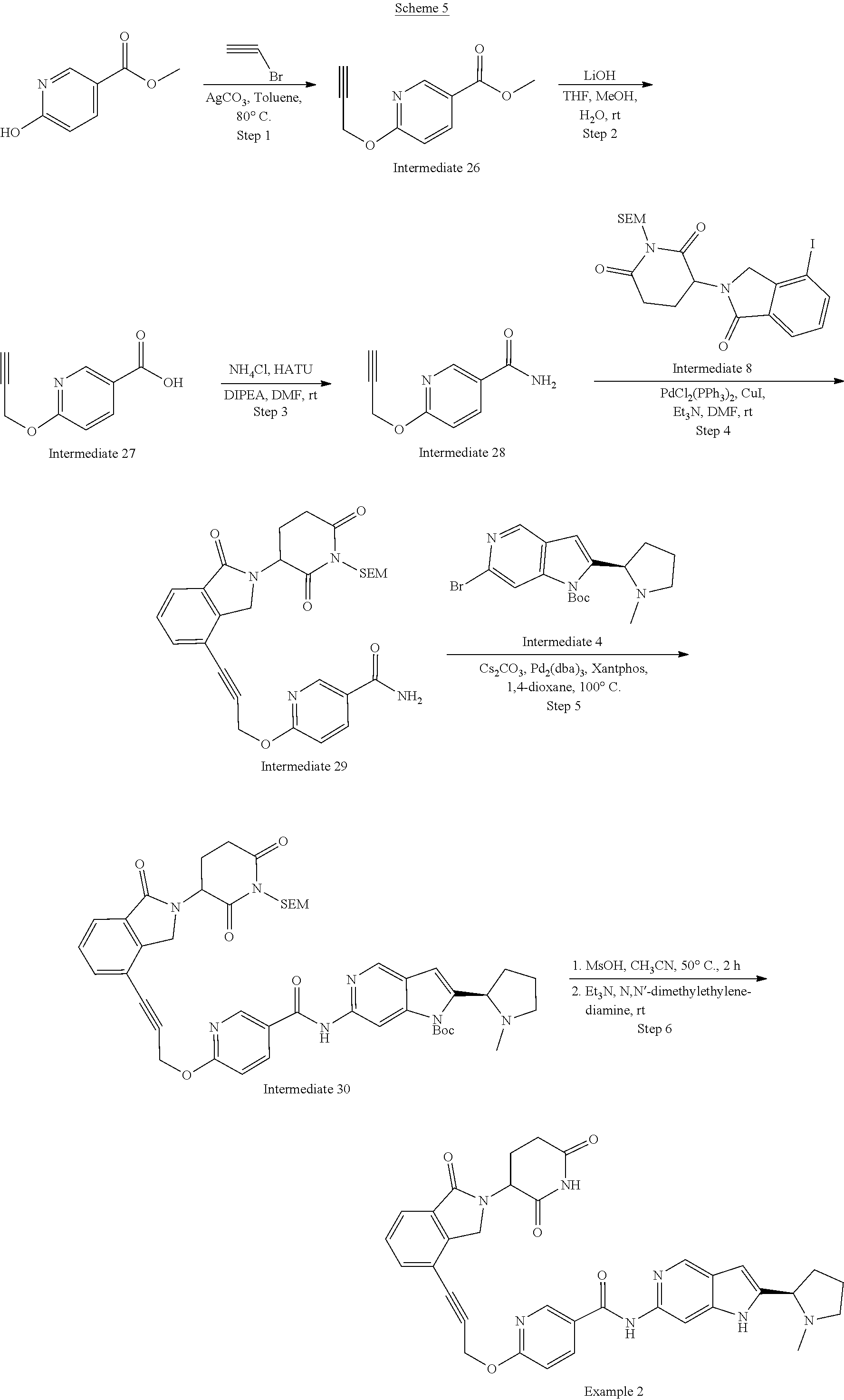

### Step 1

### Intermediate 26: methyl 6-(prop-2-yn-1-yloxy)nicotinate

To a solution of methyl 6-hydroxynicotinate (CAS No: 66171-50-4, 2.00 g, 13.060 mmol) in toluene (50 mL) was added 3-bromoprop-1-yne (CAS No: 106-96-7, 1.86 g, 15.67 mmol) followed by silver carbonate (9.47 g, 32.65 mmol) and the reaction mixture was heated at 80° C. for 48 h. The reaction mixture was diluted with water (500 mL) and extracted with ethyl acetate (3×200 mL). The combined organic phases were dried over anhydrous Na₂SO₄, filtered and concentrated in vacuo. The crude material was purified by combi-flash chromatography by eluting with 45% ethyl acetate in heptane to afford methyl 6-(prop-2-yn-1-yloxy)nicotinate (Intermediate 26, 0.50 g, 20%) as an off-white solid. ¹H NMR (400 MHz, CDCl₃) δ ppm 2.48 (s, 1H), 3.90 (s, 3H), 5.03 (s, 2H), 6.81 (d, J=8.80 Hz, 1H), 8.17 (dd, J=8.80, 2.40 Hz, 1H), 8.81 (d, J=2.40 Hz, 1H). Mass spec: m/z: 191.9 [M+H]⁺.

### Step 2

### Intermediate 27: 6-(prop-2-yn-1-yloxy) nicotinic acid

To a solution of methyl 6-(prop-2-yn-1-yloxy)nicotinate (Intermediate 26, 1.00 g, 5.23 mmol) in tetrahydrofuran (10 mL), methanol (10 mL) and water (5 mL) was added lithium hydroxide (0.51 g, 20.92 mmol) and the reaction mixture was stirred at room temperature for 16 h. The reaction mixture was diluted with water (5 mL), acidified to pH 4 with 0.5 N HCl and extracted with ethyl acetate (2×250 mL). The combined organic phases were dried over Na₂SO₄ and concentrated in vacuo to afford 6-(prop-2-yn-1-yloxy)nicotinic acid (Intermediate 27, 0.75 g, 81%) as an off-white solid, which was used for the next step without further purification. ¹H NMR (400 MHz, CDCl₃) δ ppm 2.49 (s, 1H), 5.05 (s, 2H), 6.85 (d, J=8.80 Hz, 1H), 8.22 (dd, J=8.80, 2.0 Hz, 1H), 8.88 (d, J=2.0 Hz, 1H). Mass spec: m/z: 177.9 [M+H]⁺.

### Step 3

### Intermediate 28: 6-(prop-2-yn-1-yloxy)nicotinamide

To a solution of 6-(prop-2-yn-1-yloxy)nicotinic acid (Intermediate 27, 0.75 g, 4.23 mmol) in dimethylformamide (15 mL) was added HATU (2.54 g, 6.35 mmol) and N,N-diisopropylethylamine (1.73 g, 12.70 mmol) at room temperature and stirred for 10 min.

Ammonium chloride (1.26 g, 21.16 mmol) was added at room temperature and the reaction mixture was stirred at room temperature for 16 h. The reaction mixture was quenched with ice cold water (200 mL) and extracted with ethyl acetate (3×150 mL). The combined organic phases were dried over anhydrous Na₂SO₄ and concentrated in vacuo. The crude material was purified by combi-flash chromatography by eluting with 45% ethyl acetate in heptane to afford 6-(prop-2-yn-1-yloxy)nicotinamide (Intermediate 28, 0.25 g, 34%) as a white solid. ¹H NMR (400 MHz, CDCl₃) δ ppm 2.49 (s, 1H), 5.04 (s, 2H), 6.86 (d, J=8.40 Hz, 1H), 8.07 (d, J=8.40, 2.40 Hz, 1H), 8.62 (d, J=2.40 Hz, 1H). Mass spec: m/z: 177.0 [M+H]⁺.

### Step 4

### Intermediate 29: 6-((3-(2-(2,6-dioxo-1-((2-(trimethylsilyl)ethoxy)methyl) piperidin-3-yl)-1-oxoisoindolin-4-yl) prop-2-yn-1-yl)oxy)nicotinamide

To a solution of 6-(prop-2-yn-1-yloxy)nicotinamide (Intermediate 28, 0.22 g, 1.25 mmol) in dimethylformamide (5 mL) was added 3-(4-iodo-1-oxoisoindolin-2-yl)-1-((2-(trimethylsilyl)ethoxy)methyl)piperidine-2,6-dione (Intermediate 8, 0.31 g, 0.62 mmol) followed by triethylamine (4.78 g, 44.95 mmol). The reaction mixture was purged with argon for 15 min then copper(I) iodide (0.024 g, 0.12 mmol) and PdCl₂(PPh₃)₂ (0.092 g, 0.12 mmol) were added. The reaction mixture was further purged with argon for 10 min and stirred at room temperature for 2 h. The reaction mixture was diluted with water (100 mL) and extracted with ethyl acetate (3×150 mL). The combined organic phases were dried over anhydrous Na₂SO₄ and concentrated in vacuo. The crude material was purified by combi-flash chromatography by eluting with 90% ethyl acetate in heptane to afford 6-((3-(2-(2,6-dioxo-1-((2-(trimethylsilyl)ethoxy)methyl)piperidin-3-yl)-1-oxoisoindolin-4-yl)prop-2-yn-1-yl)oxy)nicotinamide (Intermediate 29, 0.35 g, 51%) as a yellow solid. ¹H NMR (400 MHz, DMSO-d₆) δ ppm −0.03 (s, 9H), 0.80-0.88 (m, 2H), 2.02-2.11 (m, 1H), 2.30-2.41 (m, 1H), 2.76-2.82 (m, 1H), 3.01-3.12 (m, 1H), 3.49-3.57 (m, 2H), 4.18-4.24 (m, 1H), 4.38-4.43 (m, 1H), 5.02-5.09 (m, 2H), 5.24-5.28 (m, 1H), 5.32 (s, 2H), 6.97 (d, J=8.80 Hz, 1H), 7.42 (br s, 1H), 7.57 (t, J=7.60 Hz, 1H), 7.72 (d, J=7.60 Hz, 1H), 7.78 (d, J=7.60 Hz, 1H), 7.99 (br s, 1H), 8.19 (dd, J=8.80, 2.40 Hz, 1H), 8.71 (d, J=2.40 Hz, 1H). Mass spec: m/z: 546.9 [M−H]⁻.

### Step 5

### Intermediate 30: tert-butyl 6-(6-((3-(2-(2,6-dioxo-1-((2-(trimethylsilyl)ethoxy)methyl)piperidin-3-yl)-1-oxoisoindolin-4-yl)prop-2-yn-1-yl)oxy)nicotinamido)-2-((R)-1-methylpyrrolidin-2-yl)-1H-pyrrolo[3,2-c]pyridine-1-carboxylate

To a solution of 6-((3-(2-(2,6-dioxo-1-((2-(trimethylsilyl) ethoxy)methyl) piperidin-3-yl)-1-oxoisoindolin-4-yl) prop-2-yn-1-yl)oxy) nicotinamide (Intermediate 29, 0.33 g, 0.60 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.27 g, 0.72 mmol) followed by cesium carbonate (0.49 g, 1.50 mmol) at room temperature. The reaction mixture was purged with argon for 15 min then Pd₂(dba)₃ (0.085 g, 0.090 mmol) and Xantphos (0.11 g, 0.18 mmol) were added. The reaction mixture was further purged with argon for 10 min and then heated at 100° C. for 2 h. The reaction mixture was filtered through celite, washed with ethyl acetate (50 mL) and the filtrate was concentrated in vacuo to obtain crude compound. The crude material was purified by combi-flash chromatography by eluting with 90% ethyl acetate in heptane to afford tert-butyl 6-(6-((3-(2-(2,6-dioxo-1-((2-(trimethylsilyl)ethoxy)methyl)piperidin-3-yl)-1-oxoisoindolin-4-yl)prop-2-yn-1-yl)oxy)nicotinamido)-2-((R)-1-methylpyrrolidin-2-yl)-1H-pyrrolo[3,2-c]pyridine-1-carboxylate (Intermediate 30, 0.25 g, 49%) as yellow solid. ¹H NMR (400 MHz, DMSO-d₆) δ ppm −0.05 (s, 9H), 0.78-0.84 (m, 2H), 1.58-1.65 (m, 2H), 1.69 (s, 9H), 1.73-1.79 (m, 2H), 2.01-2.09 (m, 1H), 2.29-2.33 (m, 1H), 2.36 (s, 3H), 2.37-2.43 (m, 1H), 2.72-2.81 (m, 1H), 2.99-3.18 (m, 2H), 3.45-3.52 (m, 2H), 3.88-3.96 (m, 1H), 4.20-4.24 (m, 1H), 4.40-4.44 (m, 1H), 4.97-5.06 (m, 2H), 5.23-5.27 (m, 1H), 5.36 (s, 2H), 6.76 (s, 1H), 7.02 (d, J=8.40 Hz, 1H), 7.54-7.61 (m, 1H), 7.74 (d, J=7.60 Hz, 1H), 7.79 (d, J=7.60 Hz, 1H), 8.36 (dd, J=8.40, 2.80 Hz, 1H), 8.59 (s, 1H), 8.90 (s, 1H), 8.88 (d, J=2.80 Hz, 1H), 8.92 (s, 1H), 10.84 (s, 1H). Mass spec: m/z: 847.9 [M+H]⁺.

### Step 6

### Example 2: 6-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)nicotinamide

To a solution of tert-butyl 6-(6-((3-(2-(2,6-dioxo-1-((2-(trimethylsilyl)ethoxy)methyl)piperidin-3-yl)-1-oxoisoindolin-4-yl)prop-2-yn-1-yl)oxy)nicotinamido)-2-((R)-1-methylpyrrolidin-2-yl)-1H-pyrrolo[3,2-c]pyridine-1-carboxylate (Intermediate 30, 0.25 g, 0.29 mmol) in acetonitrile (10 mL) was added methane sulfonic acid (0.19 mL, 2.95 mmol) at room temperature and the reaction mixture was heated at 50° C. for 2 h. To the reaction mixture was added N,N′-dimethylethylenediamine (0.17 mL, 1.47 mmol) followed by triethylamine (0.82 mL, 5.89 mmol) and the reaction stirred at room temperature for 3 h. The reaction mixture was concentrated in vacuo and water (50 mL) added, resulting in a precipitate which was filtered and dried under vacuum to obtain crude compound. The crude material was purified by preparative HPLC (Method A) to afford 6-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)nicotinamide (Example 2, 0.045 g, 25%) as an off white solid. ¹H NMR (400 MHz, DMSO-d₆) δ ppm 1.75-1.95 (m, 3H), 1.97-2.05 (m, 1H), 2.11-2.15 (m, 1H), 2.17 (s, 3H), 2.23-2.30 (m, 1H), 2.41-2.45 (m, 1H), 2.56-2.62 (m, 1H), 2.85-2.96 (m, 1H), 3.10-3.17 (m, 1H), 3.32-3.36 (m, 1H), 4.28-4.35 (m, 1H), 4.42-4.48 (m, 1H), 5.12-5.17 (m, 1H), 5.37 (s, 2H), 6.40 (s, 1H), 7.03 (d, J=8.40 Hz, 1H), 7.54-7.59 (m, 1H), 7.71-7.75 (m, 1H), 7.76-7.80 (m, 1H), 8.19 (s, 1H), 8.36 (dd, J=8.40, 2.40 Hz, 1H), 8.51 (s, 1H), 8.89 (d, J=2.40 Hz, 1H), 10.60 (s, 1H), 11.11 (br s, 1H), 11.39 (br s, 1H). Mass spec: m/z: 618.2 [M+H]⁺.

### Example 3 was Synthesised Following Scheme 6

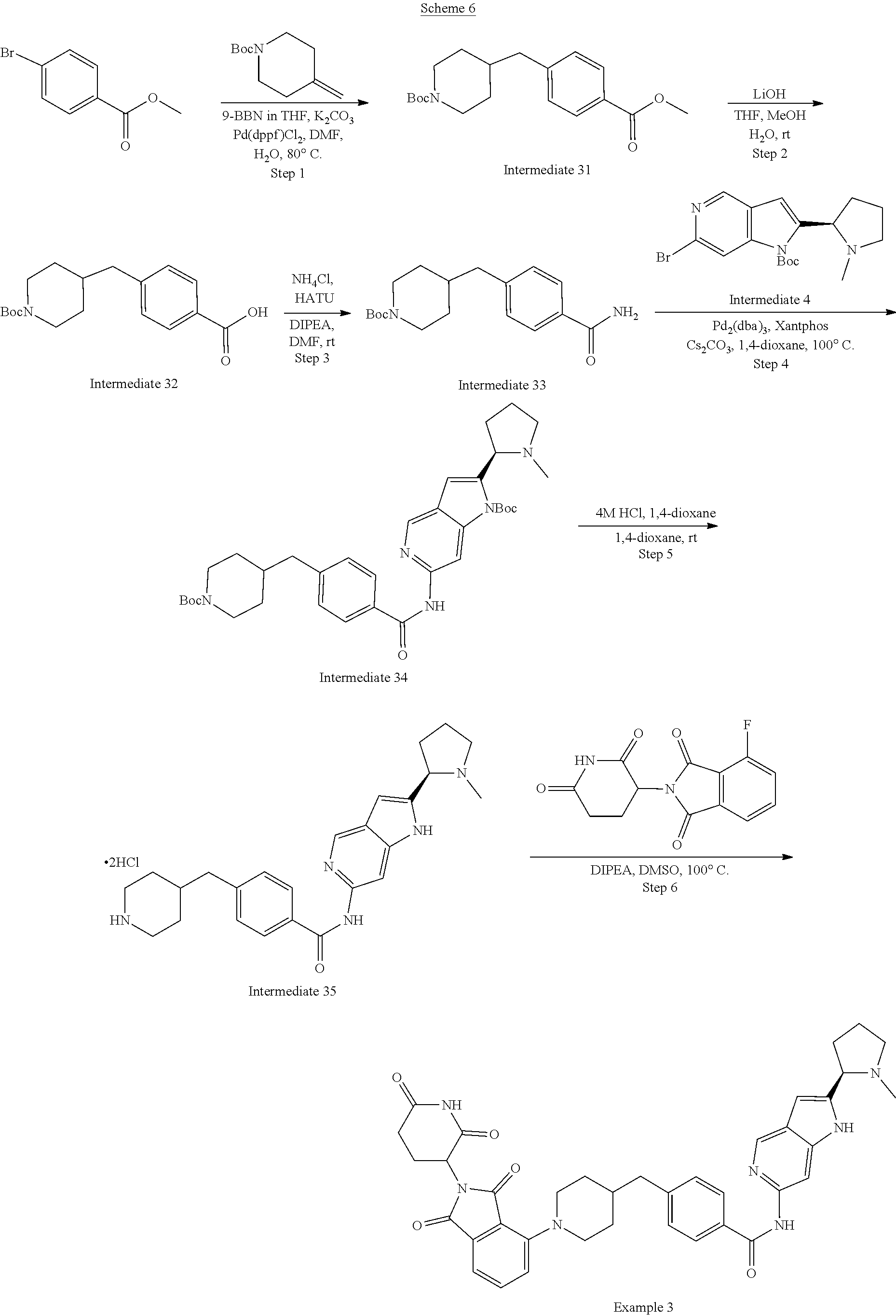

### Step 1

### Intermediate 31: tert-butyl 4-(4-(methoxycarbonyl)benzyl)piperidine-1-carboxylate

To a solution of methyl 4-bromobenzoate (CAS No: 619-42-1, 3.00 g, 13.95 mmol) in tetrahydrofuran (30 mL) was added 9-borabicyclo[3.3.1]nonane (4.26 g, 16.74 mmol) and the reaction mixture was heated at 60° C. for 1 h under a nitrogen atmosphere. tert-butyl 4-methylenepiperidine-1-carboxylate (CAS No: 159635-49-1, 3.30 g, 16.74 mmol) in dimethylformamide (20 mL) followed by potassium carbonate (3.04 g, 20.93 mmol) and PdCl₂(dppf) (1.79 g, 2.09 mmol) were added at room temperature and the reaction mixture was stirred at 80° C. for 2 h. The reaction mixture was cooled to room temperature and diluted with water (100 mL) and extracted with ethyl acetate (3×100 mL). The combined organic phases were dried over anhydrous Na₂SO₄ and concentrated in vacuo. The crude material was purified by combi-flash chromatography by eluting with 40% ethyl acetate in heptane to afford tert-butyl 4-(4-(methoxycarbonyl)benzyl) piperidine-1-carboxylate (Intermediate 31, 4.80 g, 100%) as a colorless liquid. ¹H NMR (400 MHz, DMSO-d₆) δ ppm 1.37 (s, 9H), 1.49-1.62 (m, 2H), 1.66-1.82 (m, 3H), 2.55-2.68 (m, 4H), 3.83 (s, 3H), 3.88-3.91 (m, 2H), 7.31 (d, J=7.88 Hz, 2H), 7.87 (d, J=7.88 Hz, 2H). Mass spec: m/z: 234.2 [M−100+H]⁺.

### Step 2

### Intermediate 32: 4-((1-(tert-butoxycarbonyl) piperidin-4-yl)methyl)benzoic acid

To a solution of tert-butyl 4-(4-(methoxycarbonyl)benzyl) piperidine-1-carboxylate (Intermediate 31, 4.80 g, 14.4 mmol) in tetrahydrofuran (20 mL), methanol (10 mL) and water (10 mL) was added lithium hydroxide (1.06 g, 43.2 mmol) and the reaction mixture was stirred at room temperature for 12 h. The reaction mixture was concentrated in vacuo to obtain crude compound. The crude material was diluted with water (50 mL) and acidified to pH~4 with 1N HCl (20 mL), resulting in a solid precipitate which was collected by filtration and dried to afford 4-((1-(tert-butoxycarbonyl)piperidin-4-yl)methyl)benzoic acid (Intermediate 32, 4.00 g) as a white solid, which was used for the next step without further purification. ¹H NMR (400 MHz, DMSO-d₆) δ ppm 0.98-1.04 (m, 2H), 1.37 (s, 9H), 1.50-1.54 (m, 2H), 1.67-1.70 (m, 1H), 2.57-2.62 (m, 4H), 3.87-3.91 (m, 2H), 7.28-7.30 (m, 2H), 7.84-7.86 (m, 2H), 12.78 (br s, 1H). Mass spec: m/z: 318.0 [M−H]⁻.

### Step 3

### Intermediate 33: tert-butyl 4-(4-carbamoylbenzyl) piperidine-1-carboxylate

To a solution of 4-((1-(tert-butoxycarbonyl) piperidin-4-yl)methyl)benzoic acid (Intermediate 32, 4.00 g, 12.52 mmol) in dimethylformamide (10 mL) was added HATU (7.36 g, 18.79 mmol) and the reaction mixture stirred at room temperature for 15 min. Ammonium chloride (2.68 g, 50.09 mmol) and N,N-diisopropylethylamine (8.75 mL, 50.09 mmol) were then added and the reaction mixture was stirred at room temperature for 16 h. The reaction mixture was diluted with cold water (100 mL) and extracted with ethyl acetate (3×100 mL). The combined organic phases were dried over anhydrous Na₂SO₄ and concentrated in vacuo. The crude material was purified by combi-flash chromatography by eluting with 50% ethyl acetate in heptane to afford tert-butyl 4-(4-carbamoylbenzyl) piperidine-1-carboxylate (Intermediate 33, 3.00 g, 75%) as a white solid. ¹H NMR (400 MHz, DMSO-d₆) δ ppm 0.99-1.06 (m, 2H), 1.38 (s, 9H), 1.48-1.54 (m, 2H), 1.66-1.72 (m, 1H), 2.52-2.58 (m, 4H), 3.86-3.92 (m, 2H), 7.22-7.24 (m, 3H), 7.77-7.79 (m, 2H), 7.88 (br s, 1H). Mass spec: m/z: 316.9 [M−H]⁻.

### Step 4

### Intermediate 34: tert-butyl (R)-6-(4-((1-(tert-butoxycarbonyl)piperidin-4-yl)methyl)benzamido)-2-(1-methylpyrrolidin-2-yl)-1H-pyrrolo[3,2-c]pyridine-1-carboxylate

To a solution of tert-butyl 4-(4-carbamoylbenzyl) piperidine-1-carboxylate (Intermediate 33, 0.42 g, 1.31 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.60 g, 1.64 mmol) followed by cesium carbonate (1.07 g, 3.27 mmol) at room temperature. The reaction mixture was purged with argon for 15 min then Pd₂(dba)₃ (0.15 g, 0.16 mmol) and Xantphos (0.29 g, 0.49 mmol) were added. The reaction mixture was further purged with argon for 10 min and stirred at 100° C. for 2 h. The reaction mixture was concentrated in vacuo to obtain crude compound. The crude material was purified by combi-flash chromatography by eluting with 80% ethyl acetate in heptane to afford tert-butyl (R)-6-(4-((1-(tert-butoxycarbonyl)piperidin-4-yl)methyl)benzamido)-2-(1-methylpyrrolidin-2-yl)-1H-pyrrolo[3,2-c]pyridine-1-carboxylate (Intermediate 34, 0.54 g, 53%) as a white solid. ¹H NMR (400 MHz, DMSO-d₆) δ ppm 1.01-1.09 (m, 2H), 1.38 (s, 9H), 1.53-1.62 (m, 4H), 1.69 (s, 9H), 1.74-1.80 (m, 2H), 2.25-2.31 (m, 1H), 2.35 (s, 3H), 2.38-2.40 (m, 1H), 2.54-2.61 (m, 2H), 2.65-2.72 (m, 2H), 3.11-3.16 (m, 1H), 3.88-3.93 (m, 3H), 6.74 (s, 1H), 7.30 (d, J=7.60 Hz, 2H), 7.97 (d, J=7.20 Hz, 2H), 8.58 (s, 1H), 8.92 (s, 1H), 10.58 (br s, 1H). Mass spec: m/z: 618.1 [M+H]⁺.

### Step 5

### Intermediate 35: (R)—N-(2-(1-methylpyrrolidin-2-yl)-1H-pyrrolo[3,2-c]pyridin-6-yl)-4-(piperidin-4-ylmethyl)benzamide dihydrochloride

To a solution of tert-butyl (R)-6-(4-((1-(tert-butoxycarbonyl)piperidin-4-yl)methyl)benzamido)-2-(1-methylpyrrolidin-2-yl)-1H-pyrrolo[3,2-c]pyridine-1-carboxylate (Intermediate 34, 0.52 g, 0.84 mmol) in 1,4-dioxane (8.0 mL) was added 4M hydrochloric acid in 1,4-dioxane (8.0 mL) at 0° C. and the reaction mixture was stirred at room temperature for 4 h. The reaction mixture was concentrated in vacuo to afford (R)—N-(2-(1-methylpyrrolidin-2-yl)-1H-pyrrolo[3,2-c]pyridin-6-yl)-4-(piperidin-4-ylmethyl)benzamide dihydrochloride (Intermediate 35, 0.50 g) as a white solid which was used for next step without further purification. ¹H NMR (400 MHz, DMSO-d₆) δ ppm 1.38-1.46 (m, 2H), 1.69-1.74 (m, 2H), 1.86-1.89 (m, 1H), 2.15-2.21 (m, 2H), 2.31-2.40 (m, 2H), 2.66-2.71 (m, 2H), 2.81 (s, 3H), 2.85-2.89 (m, 1H), 3.17-3.24 (m, 3H), 3.70-3.78 (m, 2H), 4.72-4.78 (m, 1H), 7.29 (s, 1H), 7.42-7.44 (m, 2H), 8.16-8.18 (m, 2H), 8.32 (s, 1H), 8.72-8.76 (m, 1H), 8.99 (br s, 1H), 9.10 (s, 1H), 11.55 (br s, 1H), 12.05 (br s, 1H), 13.33 (br s, 1H). Mass spec: m/z: 418.3 [M+H]⁺.

### Step 6

### Example 3: 4-((1-(2-(2,6-dioxopiperidin-3-yl)-1,3-dioxoisoindolin-4-yl)piperidin-4-yl)methyl)-N-(2-((R)-1-methylpyrrolidin-2-yl)-1H-pyrrolo[3,2-c]pyridin-6-yl)benzamide

To a solution of (R)—N-(2-(1-methylpyrrolidin-2-yl)-1H-pyrrolo[3,2-c]pyridin-6-yl)-4-(piperidin-4-ylmethyl)benzamide dihydrochloride (Intermediate 35, 0.40 g, 0.88 mmol) in dimethyl sulfoxide (5.0 mL) was added 2-(2,6-dioxopiperidin-3-yl)-4-fluoroisoindoline-1,3-dione (CAS No: 835616-60-9, 0.24 g, 0.88 mmol) followed by N,N-diisopropylethylamine (0.47 g, 3.52 mmol) at room temperature and the reaction mixture was heated at 100° C. for 1 h. The reaction mixture was diluted with water (50 mL), resulting in a solid precipitate which was collected by filtration and dried to obtain crude compound. The crude material was purified through reverse phase preparative HPLC (Method A) to afford 4-((1-(2-(2,6-dioxopiperidin-3-yl)-1,3-dioxoisoindolin-4-yl)piperidin-4-yl)methyl)-N-(2-((R)-1-methylpyrrolidin-2-yl)-1H-pyrrolo[3,2-c]pyridin-6-yl)benzamide (Example 3, 0.08 g, 14%) as a yellow solid. ¹H NMR (400 MHz, DMSO-d₆) δ ppm 1.40-1.48 (m, 2H), 1.65-1.74 (m, 2H), 1.76-1.95 (m, 4H), 1.99-2.05 (m, 1H), 2.12-2.15 (m, 1H), 2.17 (s, 3H), 2.23-2.35 (m, 2H), 2.56-2.62 (m, 2H), 2.66-2.70 (m, 2H), 2.81-2.88 (m, 3H), 3.12-3.16 (m, 1H), 3.65-3.72 (m, 2H), 5.06-5.11 (m, 1H), 6.39 (s, 1H), 7.31-7.36 (m, 4H), 7.65-7.69 (m, 1H), 8.00 (d, J=8.26 Hz, 2H), 8.19 (s, 1H), 8.50 (s, 1H), 10.35 (br s, 1H), 10.94 (br s, 1H), 11.35 (br s, 1H). Mass spec: m/z: 674.1 [M+H]⁺.

### Example 4 was Synthesised Following Scheme 7

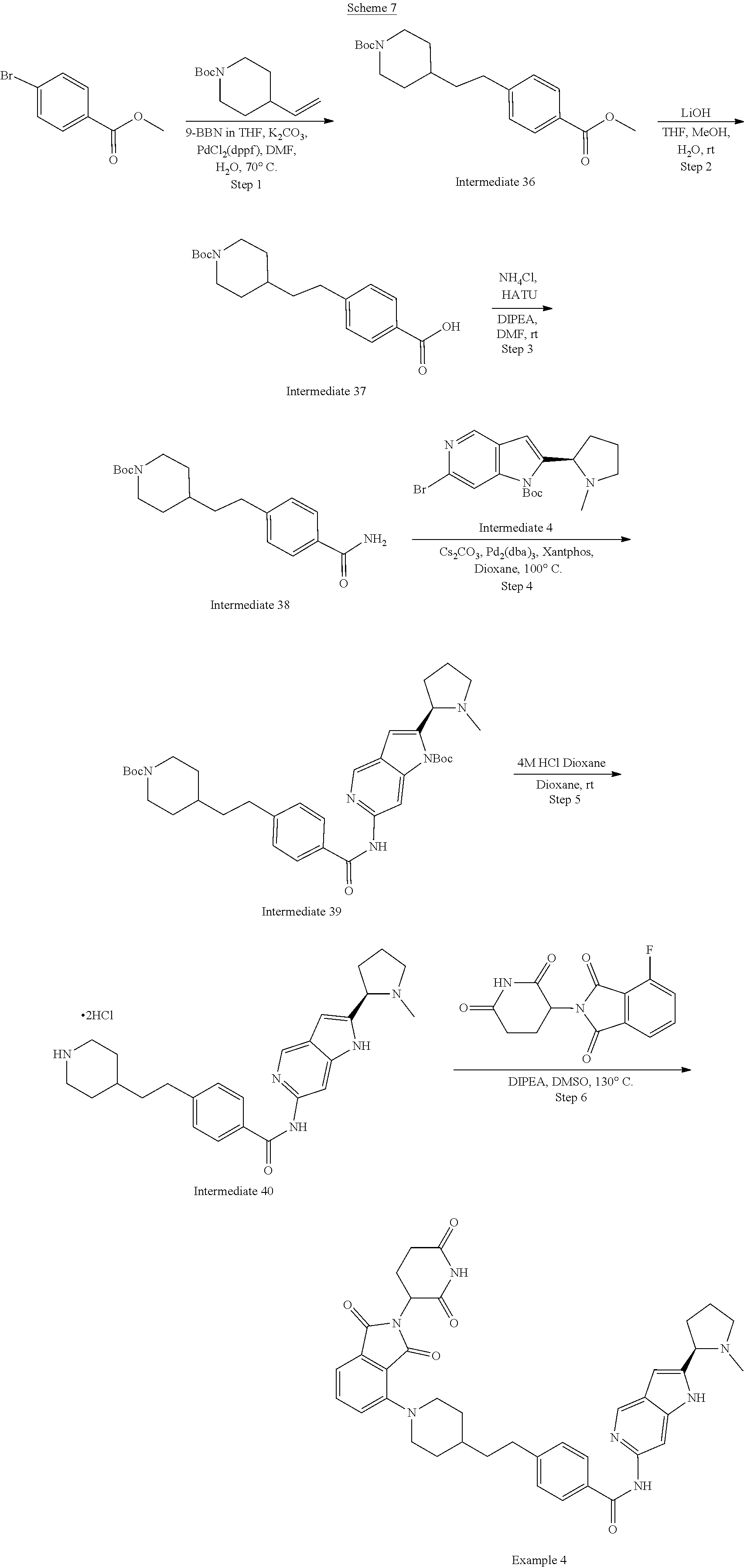

### Step 1

### Intermediate 36: tert-butyl 4-(4-(methoxycarbonyl) phenethyl) piperidine-1-carboxylate

To a solution of tert-butyl 4-vinylpiperidine-1-carboxylate (CAS No: 180307-56-6, 3.00 g, 14 mmol) in tetrahydrofuran (50 mL) was added 9-borabicyclo[3.3.1]nonane (30.0 mL, 17 mmol) at 0° C. The reaction mixture was heated at 70° C. for 2 h. The reaction mixture was cooled to room temperature. To the reaction mixture was added methyl 4-bromobenzoate (CAS No: 619-42-1, 3.70 g, 17 mmol) followed by potassium carbonate (3.1 g, 21.0 mmol) and PdCl₂(dppf) (1.6 g, 1.8 mmol) in dimethylformamide (20 mL) and water (3 mL) at room temperature. The reaction mixture was further heated at 70° C. for 3 h. Progress of the reaction was monitored by TLC. After completion, the reaction mixture was poured into water (100 mL) and extracted with ethyl acetate (3×100 mL). The combined organic phases were dried over anhydrous Na₂SO₄ and concentrated in vacuo to obtain crude compound. The crude material was purified by combi-flash chromatography by eluting with 20% ethyl acetate in heptane to afford tert-butyl 4-(4-(methoxycarbonyl) phenethyl) piperidine-1-carboxylate (Intermediate 36, 4.00 g, 82%) as a colorless solid. ¹H NMR (400 MHz, DMSO-d₆) δ ppm 0.98-1.02 (m, 2H), 1.38 (s, 9H), 1.49-1.57 (m, 2H), 1.60-1.77 (m, 3H), 2.65-2.69 (m, 4H), 3.83 (s, 3H), 3.88-3.93 (m, 2H), 7.35 (d, J=7.60 Hz, 2H), 7.87 (d, J=7.60 Hz, 2H). Mass spec: m/z: 291.9 [M−56+H]⁺.

### Step 2

### Intermediate 37: 4-(2-(1-(tert-butoxycarbonyl)piperidin-4-yl)ethyl)benzoic acid

To a solution of tert-butyl 4-(4-(methoxycarbonyl)phenethyl) piperidine-1-carboxylate (Intermediate 36, 4.00 g, 12 mmol) in tetrahydrofuran:methanol:water (1:1:1, 70 mL) was added lithium hydroxide (0.84 g, 95 mmol) at 0° C. and the reaction mixture was stirred at room temperature for 16 h. The reaction mixture was concentrated in vacuo and water (100 mL) added and the mixture acidified with 1N HCl, resulting in a precipitate which was filtered and dried to afford 4-(2-(1-(tert-butoxycarbonyl)piperidin-4-yl)ethyl)benzoic acid (Intermediate 37, 2.50 g) as an off white solid, which was used for the next step without further purification. ¹H NMR (400 MHz, DMSO-d₆) δ ppm 0.98-1.02 (m, 2H), 1.38 (s, 9H), 1.46-1.48 (m, 1H), 1.49-1.55 (m, 2H), 1.65-1.70 (m, 2H), 2.62-2.70 (m, 4H), 3.88-3.94 (m, 2H), 7.32 (d, J=8.0 Hz, 2H), 7.85 (d, J=8.0 Hz, 2H), 12.78 (br s, 1H). Mass spec: m/z: 278.0 [M−56+H]⁺.

### Step 3

### Intermediate 38: tert-butyl 4-(4-carbamoylphenethyl)piperidine-1-carboxylate

To a solution of 4-(2-(1-(tert-butoxycarbonyl) piperidin-4-yl)ethyl)benzoic acid (Intermediate 37, 2.50 g, 7.5 mmol) in dimethylformamide (30 mL) was added HATU (4.4 g, 11 mmol) and N,N-diisopropylethylamine (4.0 g, 30 mmol) at 0° C. and the reaction stirred for 10 min. Ammonium chloride (1.6 g, 30.0 mmol) was then added at 0° C. and the reaction mixture was stirred at room temperature for 16 h. The reaction mixture was poured into water (100 mL), resulting in a precipitate, which was filtered and dried under vacuum to afford tert-butyl 4-(4-carbamoylphenethyl) piperidine-1-carboxylate (Intermediate 38, 1.80 g, 72%) as an off white solid, which was used for next step without further purification. ¹H NMR (400 MHz, DMSO-d₆) δ ppm 0.94-1.06 (m, 2H), 1.39 (s, 9H), 1.46-1.48 (m, 1H), 1.49-1.54 (m, 2H), 1.66-1.69 (m, 2H), 2.61-2.68 (m, 4H), 3.88-3.94 (m, 2H), 7.23 (br s, 1H), 7.27 (d, J=7.60 Hz, 2H), 7.78 (d, J=7.60 Hz, 2H), 7.86 (br s, 1H). Mass spec: m/z: 331.3 [M−H]⁻.

### Step 4

### Intermediate 39: tert-butyl (R)-6-(4-(2-(1-(tert-butoxycarbonyl) piperidin-4-yl)ethyl)benzamido)-2-(1-methylpyrrolidin-2-yl)-1H-pyrrolo[3,2-c]pyridine-1-carboxylate

To a solution of tert-butyl 4-(4-carbamoylphenethyl)piperidine-1-carboxylate (Intermediate 38, 0.27 g, 0.84 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 0.40 g, 1.05 mmol) followed by cesium carbonate (0.68 g, 2.10 mmol) at room temperature. The reaction mixture was purged with argon for 15 min followed by the addition of Pd₂(dba)₃ (0.15 g, 0.00157 mmol) and Xantphos (0.18 g, 0.31 mmol). The reaction mixture was further purged with argon for 10 min and then heated at 100° C. for 2 h. The reaction mixture was concentrated in vacuo to obtain crude compound which purified by combi-flash chromatography by eluting with 80% ethyl acetate in heptane to afford tert-butyl (R)-6-(4-(2-(1-(tert-butoxycarbonyl)piperidin-4-yl)ethyl)benzamido)-2-(1-methylpyrrolidin-2-yl)-1H-pyrrolo[3,2-c]pyridine-1-carboxylate (Intermediate 39, 0.45 g, 68%) as a yellow solid. ¹H NMR (400 MHz, DMSO-d₆) δ ppm 1.35-1.38 (m, 2H), 1.40 (s, 9H), 1.41-1.45 (m, 2H), 1.52-1.68 (m, 4H), 1.70 (s, 9H), 1.72-1.78 (m, 2H), 2.31-2.34 (m, 2H), 2.36 (s, 3H), 2.64-2.69 (m, 4H), 3.12-3.16 (m, 1H), 3.90-3.96 (m, 3H), 6.75 (s, 1H), 7.31-7.34 (m, 2H), 7.96-7.98 (m, 2H), 8.59 (s, 1H), 8.93 (s, 1H), 10.59 (br s, 1H). Mass spec: m/z: 532.4 [M−100+H]⁺.

### Step 5

### Intermediate 40: (R)—N-(2-(1-methylpyrrolidin-2-yl)-1H-pyrrolo[3,2-c]pyridin-6-yl)-4-(2-(piperidin-4-yl)ethyl)benzamide dihydrochloride

To a solution of tert-butyl (R)-6-(4-(2-(1-(tert-butoxycarbonyl)piperidin-4-yl)ethyl)benzamido)-2-(1-methylpyrrolidin-2-yl)-1H-pyrrolo[3,2-c]pyridine-1-carboxylate (Intermediate 39, 0.40 g, 0.63 mmol) in 1,4-dioxane (5 mL) was added 4M hydrochloric acid in 1,4-dioxane (5 mL) at 0° C. and the reaction mixture was stirred at room temperature for 16 h. The reaction mixture was concentrated in vacuo to afford as (R)—N-(2-(1-methylpyrrolidin-2-yl)-1H-pyrrolo[3,2-c]pyridin-6-yl)-4-(2-(piperidin-4-yl)ethyl)benzamide dihydrochloride (Intermediate 40, 0.40 g) as a brown solid, which was used for next step without further purification. ¹H NMR (400 MHz, DMSO-d₆) δ ppm 1.34-1.39 (m, 2H), 1.52-1.59 (m, 3H), 1.84-1.87 (m, 2H), 2.10-2.20 (m, 2H), 2.32-2.40 (m, 1H), 2.71-2.76 (m, 3H), 2.81 (s, 3H), 3.22-3.26 (m, 3H), 3.54-3.57 (m, 1H), 3.72-3.78 (m, 2H), 4.70-4.76 (m, 1H), 7.29 (s, 1H), 7.44-7.46 (m, 2H), 8.12-8.16 (m, 2H), 8.30 (s, 1H), 8.69-8.78 (m, 1H), 8.94 (s, 1H), 9.10 (s, 1H), 11.54 (br s, 1H), 12.04 (br s, 1H), 13.34 (br s, 1H). Mass spec: m/z: 432.0 [M+H]⁺.

### Step 6

### Example 4: 4-(2-(1-(2-(2,6-dioxopiperidin-3-yl)-1,3-dioxoisoindolin-4-yl)piperidin-4-yl)ethyl)-N-(2-((R)-1-methylpyrrolidin-2-yl)-1H-pyrrolo[3,2-c]pyridin-6-yl)benzamide

To a solution of (R)—N-(2-(1-methylpyrrolidin-2-yl)-1H-pyrrolo[3,2-c]pyridin-6-yl)-4-(2-(piperidin-4-yl)ethyl)benzamide dihydrochloride (Intermediate 40, 0.38 g, 0.88 mmol) in dimethyl sulfoxide (5 mL) was added 2-(2,6-dioxopiperidin-3-yl)-4-fluoroisoindoline-1,3-dione (CAS No: 835616-60-9, 0.24 g, 0.88 mmol) followed by N,N-diisopropylethylamine (0.58 g, 4.40 mmol) at room temperature and the reaction mixture was heated at 130° C. for 2 h. The reaction mixture was poured into water (10 mL), resulting in a precipitate which was filtered and dried under vacuum to obtain the crude compound. The crude material was purified by preparative HPLC (Method A) to afford 4-(2-(1-(2-(2,6-dioxopiperidin-3-yl)-1,3-dioxoisoindolin-4-yl)piperidin-4-yl)ethyl)-N-(2-((R)-1-methylpyrrolidin-2-yl)-1H-pyrrolo[3,2-c]pyridin-6-yl)benzamide (Example 4, 0.13 g, 22%) as a yellow solid. ¹H NMR (400 MHz, DMSO-d₆) δ ppm 1.39-1.50 (m, 3H), 1.55-1.68 (m, 2H), 1.78-1.92 (m, 5H), 2.01-2.04 (m, 1H), 2.06-2.12 (m, 1H), 2.16 (s, 3H), 2.23-2.32 (m, 1H), 2.52-2.61 (m, 3H), 2.71-2.75 (m, 2H), 2.82-2.88 (m, 3H), 3.12-3.16 (m, 1H), 3.68-3.73 (m, 2H), 5.07-5.11 (m, 1H), 6.39 (s, 1H), 7.31-7.37 (m, 4H), 7.66-7.69 (m, 1H), 7.99 (d, J=8.31 Hz, 2H), 8.19 (s, 1H), 8.50 (s, 1H), 10.36 (s, 1H), 11.36 (s, 1H). Mass spec: m/z: 688.3 [M+H]⁺.

### Example 5 was Synthesised Following Scheme 8

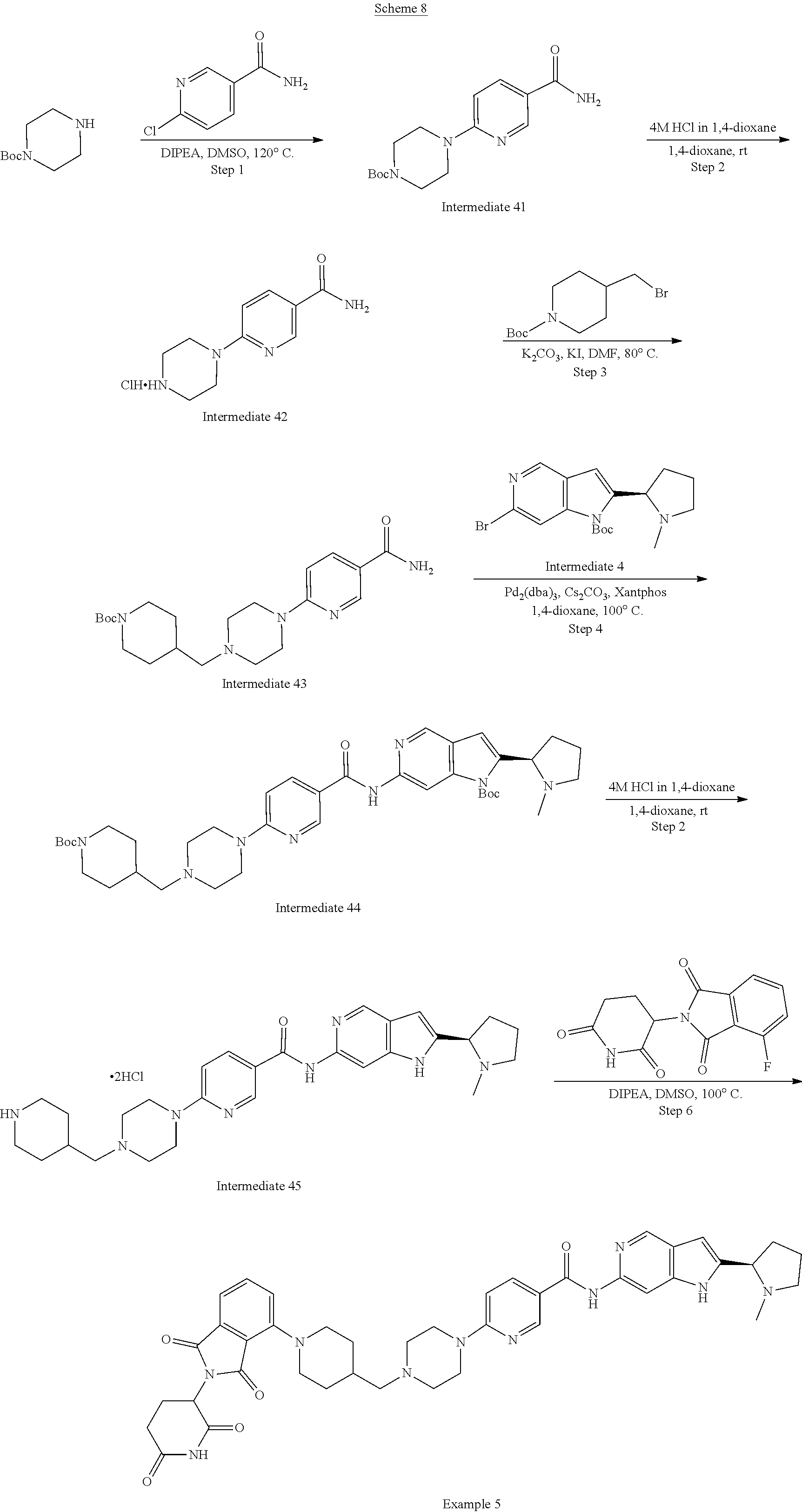

### Step 1

### Intermediate 41: tert-butyl 4-(5-carbamoylpyridin-2-yl) piperazine-1-carboxylate

To a solution of tert-butyl piperazine-1-carboxylate (CAS No: 57260-71-6, 5.00 g, 26.8 mmol) in dimethyl sulfoxide (60 mL) was added 6-chloronicotinamide (CAS No: 6271-78-9, 4.20 g, 26.8 mmol) followed by N,N-diisopropylethylamine (14.1 mL, 80.5 mmol) and the reaction mixture was heated at 120° C. for 16 h. The reaction mixture was poured into ice cold water (500 mL), resulting in a precipitate, which was filtered, washed with cold water (2×150 mL), heptane (2×100 mL) and dried in vacuo to afford tert-butyl 4-(5-carbamoylpyridin-2-yl) piperazine-1-carboxylate (Intermediate 41, 4.00 g, 49%) as a yellow solid, which was used for the next step without further purification. ¹H NMR (400 MHz, DMSO-d₆) δ ppm 1.42 (s, 9H), 3.38-3.46 (m, 4H), 3.54-3.65 (m, 4H), 6.84 (d, J=9.20 Hz, 1H), 7.14 (br s, 1H), 7.77 (br s, 1H), 7.89 (d, J=9.20 Hz, 1H), 8.62 (s, 1H).

### Step 2

### Intermediate 42: 6-(piperazin-1-yl)nicotinamide hydrochloride

To a solution of tert-butyl 4-(5-carbamoylpyridin-2-yl) piperazine-1-carboxylate (Intermediate 41, 3.50 g, 11.0 mmol) in 1,4-dioxane (60 mL) was added 4M hydrochloric acid in 1,4-dioxane (32 mL) at 0° C. and the reaction mixture was stirred at room temperature for 4 h. The reaction mixture was concentrated in vacuo, washed with ether (2×20 mL) and dried under vacuum to afford as 6-(piperazin-1-yl)nicotinamide hydrochloride (Intermediate 42, 2.80 g) as a white solid, which was used for the next step without further purification. ¹H NMR (400 MHz, DMSO-d₆) δ ppm 3.88-4.02 (m, 4H), 2.52-2.55 (m, 4H), 7.14 (d, J=9.20 Hz, 1H), 7.36 (br s, 1H), 8.02 (br s, 1H), 8.18 (d, J=9.20 Hz, 1H), 8.62 (s, 1H), 9.55 (br s, 1H), 9.58 (br s, 1H). Mass spec: m/z: 207.5 [M+H]⁺.

### Step 3

### Intermediate 43: tert-butyl 4-((4-(5-carbamoylpyridin-2-yl) piperazin-1-yl)methyl) piperidine-1-carboxylate

To a solution 6-(piperazin-1-yl)nicotinamide hydrochloride (Intermediate 42, 2.80 g, 11.5 mmol) in dimethylformamide (60 mL) was added tert-butyl 4-(bromomethyl)piperidine-1-carboxylate (CAS No: 158407-04-6, 3.85 g, 13.8 mmol) followed by potassium iodide (0.19 g, 1.15 mmol) and potassium carbonate (7.97 g, 57.7 mmol) and the reaction mixture was heated at 80° C. for 16 h. The reaction mixture was poured into ice cold water (200 mL), resulting in a precipitate which filtered, washed with water (80 mL), n-pentane (80 mL) and then dried in under vacuum to afford tert-butyl 4-((4-(5-carbamoylpyridin-2-yl) piperazin-1-yl)methyl) piperidine-1-carboxylate (Intermediate 43, 0.80 g, 17%) as an off white solid, which was used for the next step without further purification. ¹H NMR (400 MHz, DMSO-d₆) δ ppm 0.99-1.14 (m, 3H) 1.38 (s, 9H), 1.66-1.76 (m, 4H), 2.60-2.81 (m, 4H), 3.50-3.63 (m, 4H), 3.86-4.02 (m, 4H), 6.81 (d, J=9.20 Hz, 1H), 7.11 (br s, 1H), 7.74 (br s, 1H), 7.94 (d, J=9.20 Hz, 1H), 8.59 (s, 1H). Mass spec: m/z: 404.3 [M+H]⁺.

### Step 4

### Intermediate 44: tert-butyl (R)-6-(6-(4-((1-(tert-butoxycarbonyl)piperidin-4-yl)methyl)piperazin-1-yl) nicotinamido)-2-(1-methylpyrrolidin-2-yl)-1H-pyrrolo[3,2-c]pyridine-1-carboxylate

To a solution of tert-butyl 4-((4-(5-carbamoylpyridin-2-yl)piperazin-1-yl)methyl)piperidine-1-carboxylate (Intermediate 43, 0.62 g, 1.53 mmol) in 1,4-dioxane (15 mL) were added tert-butyl (R)-6-bromo-2-(1-methylpyrrolidin-2-yl)-1H-pyrrolo[3,2-c]pyridine-1-carboxylate (Intermediate 4, 0.64 g, 1.69 mmol) and cesium carbonate (1.25 g, 3.84 mmol) at room temperature. The reaction mixture was purged with argon for 15 min, then Pd₂(dba)₃ (0.22 g, 0.23 mmol) and Xantphos (0.27 g, 0.46 mmol) were added. The reaction mixture was further purged with argon for 10 min and then heated at 100° C. for 2 h. The reaction mixture was filtered through celite, washed with ethyl acetate (100 mL) and the filtrate was concentrated in vacuo to obtain the crude compound. The crude material was purified by combi-flash chromatography by eluting with 5% MeOH in DCM to afford tert-butyl (R)-6-(6-(4-((1-(tert-butoxycarbonyl)piperidin-4-yl)methyl)piperazin-1-yl)nicotinamido)-2-(1-methylpyrrolidin-2-yl)-1H-pyrrolo[3,2-c]pyridine-1-carboxylate (Intermediate 44, 0.32 g, 30%) as a yellow solid. ¹H NMR (400 MHz, DMSO-d₆) δ ppm 0.90-1.03 (m, 2H), 1.39 (s, 9H), 1.52-1.64 (m, 2H), 1.68 (s, 9H), 1.71-1.80 (m, 4H), 2.11-2.20 (m, 2H), 2.34 (s, 3H), 2.37-2.44 (m, 4H), 2.66-2.72 (m, 3H), 3.08-3.14 (m, 1H), 3.56-3.68 (m, 4H), 3.84-3.98 (m, 4H), 6.73 (s, 1H), 6.86 (d, J=9.20 Hz, 1H), 8.15 (d, J=9.20 Hz, 1H), 8.56 (s, 1H), 8.79 (s, 1H), 8.89 (s, 1H), 10.48 (br s, 1H). Mass spec: m/z: 703.1 [M+H]⁺.

### Step 5

### Intermediate 45: (R)—N-(2-(1-methylpyrrolidin-2-yl)-1H-pyrrolo[3,2-c]pyridin-6-yl)-6-(4-(piperidin-4-ylmethyl) piperazin-1-yl) nicotinamide dihydrochloride

To a solution of tert-butyl (R)-6-(6-(4-((1-(tert-butoxycarbonyl)piperidin-4-yl)methyl)piperazin-1-yl) nicotinamido)-2-(1-methylpyrrolidin-2-yl)-1H-pyrrolo[3,2-c]pyridine-1-carboxylate (Intermediate 44, 0.30 g, 0.42 mmol) in 1,4-dioxane (10 mL) was added 4M hydrochloric acid in 1,4-dioxane (5 mL) and the reaction mixture was stirred at room temperature for 4 h. The reaction mixture was concentrated in vacuo, the resulting residue was washed with diethyl ether (2×50 mL) and dried under reduced pressure to afford (R)—N-(2-(1-methylpyrrolidin-2-yl)-1H-pyrrolo[3,2-c]pyridin-6-yl)-6-(4-(piperidin-4-ylmethyl) piperazin-1-yl) nicotinamide dihydrochloride (Intermediate 45, 0.21 g) as an off-white solid, which was used for the next step without further purification. Mass spec: m/z: 503.34 [M+H]⁺.

### Step 6

### Example 5: 6-(4-((1-(2-(2,6-dioxopiperidin-3-yl)-1,3-dioxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)nicotinamide

To a solution of (R)—N-(2-(1-methylpyrrolidin-2-yl)-1H-pyrrolo[3,2-c]pyridin-6-yl)-6-(4-(piperidin-4-ylmethyl)piperazin-1-yl)nicotinamide dihydrochloride (Intermediate 45, 0.21 g, 0.42 mmol) in dimethyl sulfoxide (10 mL) was added 2-(2,6-dioxopiperidin-3-yl)-4-fluoroisoindoline-1,3-dione (CAS No: 835616-60-9, 0.11 g, 0.42 mmol) followed by N,N-Diisopropylethylamine (0.36 mL, 2.08 mmol) and the reaction mixture was heated at 100° C. for 4 h. The reaction mixture was concentrated in vacuo. The obtained residue was poured into ice cold water (10 mL), resulting in a precipitate, which was filtered and dried under vacuum to obtain the crude compound. The crude material was purified by preparative HPLC (Method A) to afford 6-(4-((1-(2-(2,6-dioxopiperidin-3-yl)-1,3-dioxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)nicotinamide (Example 5, 0.03 g, 9%) as a yellow solid. ¹H NMR (400 MHz, DMSO-d₆) δ ppm 1.29-1.41 (m, 2H), 1.73-1.95 (m, 6H), 1.98-2.08 (m, 1H), 2.09-2.14 (m, 1H), 2.16 (s, 3H), 2.22-2.29 (m, 3H), 2.43-2.47 (m, 4H), 2.57-2.62 (m, 2H), 2.82-2.95 (m, 3H), 3.12-3.16 (m, 1H), 3.28-3.30 (m, 1H), 3.60-3.68 (m, 4H), 3.68-3.75 (m, 2H), 5.07-5.12 (m, 1H), 6.38 (s, 1H), 6.88 (d, J=9.20 Hz, 1H), 7.29-7.37 (m, 2H), 7.66-7.70 (m, 1H), 8.14-8.19 (m, 2H), 8.48 (s, 1H), 8.80 (s, 1H), 10.30 (s, 1H), 11.09 (br s, 1H), 11.35 (s, 1H). Mass spec: m/z: 759.2 [M+H]⁺.

### Example 6 was Synthesised Following Scheme 9

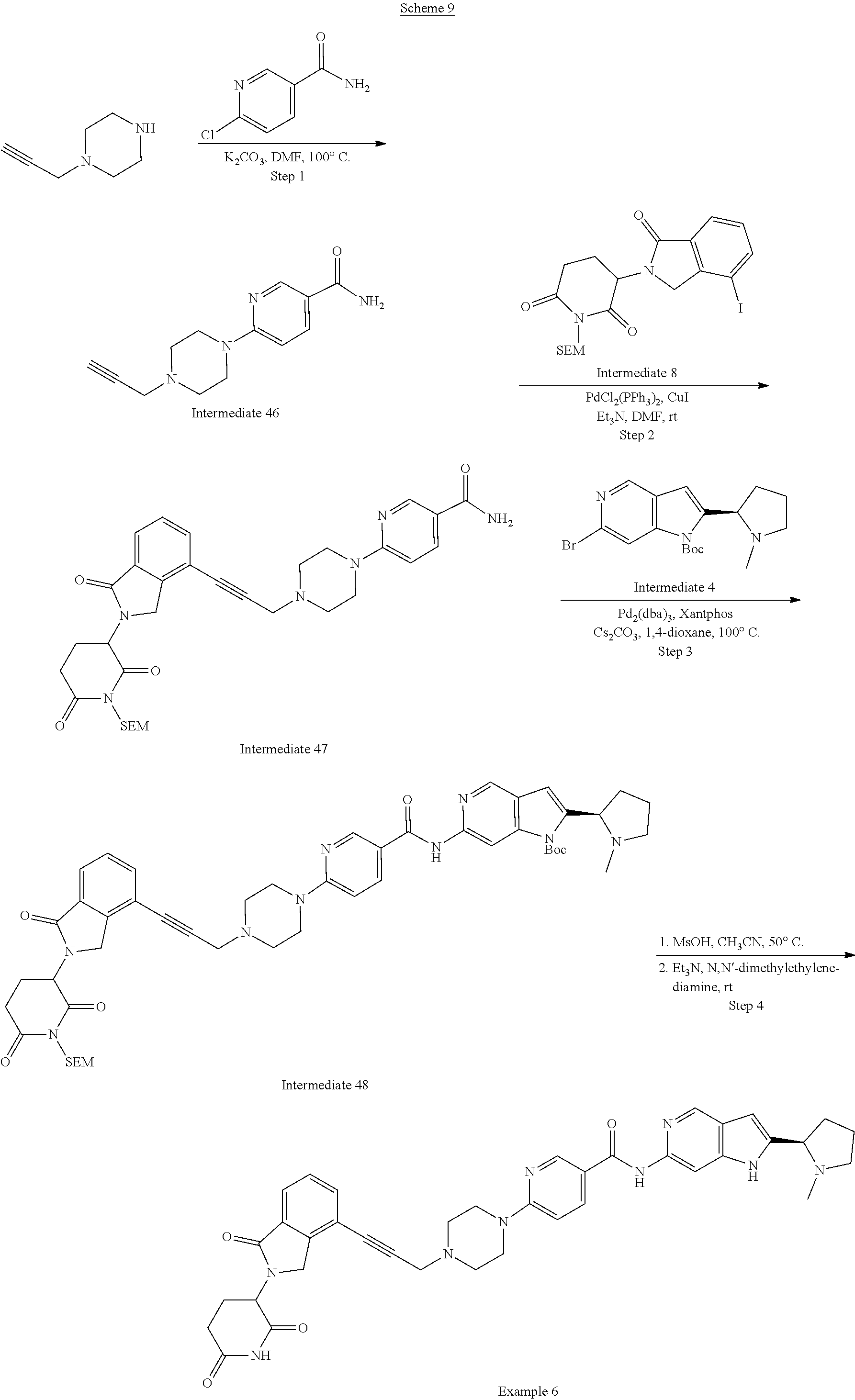

### Step 1

### Intermediate 46: 6-(4-(prop-2-yn-1-yl)piperazin-1-yl)nicotinamide

To a solution of 1-(prop-2-yn-1-yl)piperazine (CAS No: 52070-67-4, 1.50 g, 12 mmol) in dimethylformamide (10 mL) was added 6-chloronicotinamide (CAS No: 6271-78-9, 2.26 g, 14.48 mmol) followed by potassium carbonate (6.00 g, 60 mmol) and the reaction mixture was stirred at 100° C. for 12 h. The reaction mixture was diluted with water (200 mL) and extracted with ethyl acetate (3×200 mL). The combined organic phases were dried over anhydrous Na₂SO₄ and concentrated in vacuo. The crude material was purified by combi-flash column chromatography by eluting with 22% ethyl acetate in heptane to afford 6-(4-(prop-2-yn-1-yl) piperazin-1-yl) nicotinamide (Intermediate 46, 0.60 g, 20%) as an off white solid. ¹H NMR (400 MHz, DMSO-d₆) δ ppm 2.42-2.49 (m, 2H), 2.52-2.57 (m, 2H), 3.16 (s, 1H), 3.55-3.67 (m, 6H), 6.84 (d, J=9.0 Hz, 1H), 7.12 (br s, 1H), 7.75 (br s, 1H), 7.95 (d, J=9.0 Hz, 1H), 8.60 (s, 1H). Mass spec: m/z: 245.13 [M+H]⁺.

### Step 2

### Intermediate 47: 6-(4-(3-(2-(2,6-dioxo-1-((2-(trimethylsilyl)ethoxy)methyl)piperidin-3-yl)-1-oxoisoindolin-4-yl)prop-2-yn-1-yl) piperazin-1-yl)nicotinamide

To a solution of 6-(4-(prop-2-yn-1-yl)piperazin-1-yl)nicotinamide (Intermediate 46, 0.60 g, 2 mmol) in dimethylformamide (10 mL) was added 3-(4-iodo-1-oxoisoindolin-2-yl)-1-((2-(trimethylsilyl)ethoxy)methyl)piperidine-2,6-dione (Intermediate 8, 1.00 g, 2 mmol) followed by triethylamine (10 mL, 90 mmol). The reaction mixture was purged with argon for 15 min and then copper(I) iodide (0.05 g, 0.2 mmol) and PdCl₂(PPh₃)₂ (0.20 g, 0.2 mmol) were added. The reaction mixture was further purged with argon for 10 min and stirred at room temperature for 3 h. The reaction mixture was diluted with water (100 mL) and extracted with ethyl acetate (2×100 mL). The combined organic phases were dried over anhydrous Na₂SO₄, and concentrated in vacuo. The crude material was purified by combi-flash chromatography by eluting with 100% ethyl acetate to afford 6-(4-(3-(2-(2,6-dioxo-1-((2-(trimethylsilyl)ethoxy)methyl)piperidin-3-yl)-1-oxoisoindolin-4-yl) prop-2-yn-1-yl)piperazin-1-yl)nicotinamide (Intermediate 47, 0.84 g, 60%) as an yellow solid. ¹H NMR (401 MHz, DMSO-d₆) δ ppm −0.02 (s, 9H), 0.74-0.89 (m, 2H), 2.01-2.10 (m, 1H), 2.38-2.47 (m, 1H), 2.58-2.68 (m, 4H), 2.73-2.83 (m, 1H), 3.00-3.12 (m, 1H), 3.49-3.57 (m, 2H), 3.62-3.68 (m, 6H), 4.28-4.32 (m, 1H), 4.47-4.51 (m, 1H), 5.01-5.08 (m, 2H), 5.23-5.28 (m, 1H), 6.85 (d, J=9.05 Hz, 1H), 7.16 (br s, 1H), 7.53-7.58 (m, 1H), 7.70-7.72 (m, 1H), 7.74-7.78 (m, 2H), 7.96 (dd, J=9.05, 2.32 Hz, 1H), 8.61 (d, J=2.32 Hz, 1H). Mass spec: m/z: 617.42 [M+H]⁺.

### Step 3

### Intermediate 48: tert-butyl 6-(6-(4-(3-(2-(2,6-dioxo-1-((2-(trimethylsilyl)ethoxy)methyl)piperidin-3-yl)-1-oxoisoindolin-4-yl)prop-2-yn-1-yl)piperazin-1-yl)nicotinamido)-2-((R)-1-methylpyrrolidin-2-yl)-1H-pyrrolo[3,2-c]pyridine-1-carboxylate

To a solution of 6-(4-(3-(2-(2,6-dioxo-1-((2-(trimethylsilyl)ethoxy)methyl)piperidin-3-yl)-1-oxoisoindolin-4-yl) prop-2-yn-1-yl) piperazin-1-yl)nicotinamide (Intermediate 47, 0.40 g, 0.6 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.20 g, 0.6 mmol) followed by cesium carbonate (0.4 g, 1 mmol) at room temperature. The reaction mixture was purged with argon for 15 min and then Pd₂(dba)₃ (0.06 g, 0.06 mmol) and Xantphos (0.1 g, 0.2 mmol) were added. The reaction mixture was further purged with argon for 10 min and stirred at 100° C. for 2 h. The reaction mixture was concentrated in vacuo to obtain the crude compound, which was purified by combi-flash chromatography by eluting with 90% ethyl acetate in heptane to afford tert-butyl 6-(6-(4-(3-(2-(2,6-dioxo-1-((2-(trimethylsilyl)ethoxy)methyl)piperidin-3-yl)-1-oxoisoindolin-4-yl)prop-2-yn-1-yl)piperazin-1-yl)nicotinamido)-2-((R)-1-methylpyrrolidin-2-yl)-1H-pyrrolo[3,2-c]pyridine-1-carboxylate (Intermediate 48, 0.54 g, 90%) as a white solid. ¹H NMR (401 MHz, DMSO-d₆) δ ppm −0.03 (s, 9H), 0.80-0.86 (m, 2H), 1.38-1.42 (m, 1H), 1.53-1.65 (m, 2H), 1.69 (s, 9H), 1.73-1.80 (m, 2H), 2.02-2.10 (m, 1H), 2.36 (s, 3H), 2.38-2.41 (m, 2H), 2.62-2.69 (m, 4H), 2.74-2.83 (m, 1H), 3.08-3.18 (m, 1H), 3.48-3.56 (m, 2H), 3.65-3.73 (m, 6H), 3.86-3.95 (m, 1H), 4.29-4.37 (m, 1H), 4.44-4.51 (m, 1H), 5.10-5.15 (m, 2H), 5.22-5.29 (m, 1H), 6.74 (s, 1H), 6.90 (d, J=9.20 Hz, 1H), 7.54-7.58 (m, 1H), 7.70-7.73 (m, 1H), 7.74-7.78 (m, 1H), 8.16 (dd, J=9.20, 2.45 Hz, 1H), 8.58 (s, 1H), 8.81 (d, J=2.45 Hz, 1H), 8.92 (s, 1H), 10.54 (s, 1H). Mass spec: m/z: 916.5 [M+H]⁺.

### Step 4

### Example 6: 6-(4-(3-(2-(2,6-dioxopiperidin-3-yl)-1-oxoisoindolin-4-yl)prop-2-yn-1-yl)piperazin-1-yl)-N-(2-((R)-1-methylpyrrolidin-2-yl)-1H-pyrrolo[3,2-c]pyridin-6-yl)nicotinamide

To a solution of tert-butyl 6-(6-(4-(3-(2-(2,6-dioxo-1-((2-(trimethylsilyl)ethoxy)methyl)piperidin-3-yl)-1-oxoisoindolin-4-yl)prop-2-yn-1-yl)piperazin-1-yl)nicotinamido)-2-((R)-1-methylpyrrolidin-2-yl)-1H-pyrrolo[3,2-c]pyridine-1-carboxylate (Intermediate 48, 0.40 g, 0.4 mmol) in acetonitrile (10 mL) was added methanesulfonic acid (0.4 mL, 7 mmol) and the reaction mixture was stirred at 50° C. for 2 h. N,N′-dimethylethylenediamine (0.3 mL, 3 mmol) followed by triethylamine (0.9 g, 9 mmol) were then added and the reaction mixture stirred at room temperature for 3 h. The reaction mixture was poured into ice cold water (100 mL), resulting in a precipitate, which was collected by filtration and dried under vacuum to obtain crude compound. The crude compound was purified by preparative HPLC (Method A) to afford 6-(4-(3-(2-(2,6-dioxopiperidin-3-yl)-1-oxoisoindolin-4-yl)prop-2-yn-1-yl)piperazin-1-yl)-N-(2-((R)-1-methylpyrrolidin-2-yl)-1H-pyrrolo[3,2-c]pyridin-6-yl)nicotinamide (Example 6, 0.19 g, 60%) as an off white solid. ¹H NMR (400 MHz, DMSO-d₆) δ ppm 1.76-1.96 (m, 3H), 1.96-2.04 (m, 1H), 2.17 (s, 3H), 2.21-2.31 (m, 2H), 2.41-2.46 (m, 1H), 2.55-2.68 (m, 6H), 2.84-2.95 (m, 1H), 3.12-3.16 (m, 1H), 3.65-3.73 (m, 6H), 4.29-4.37 (m, 1H), 4.44-4.51 (m, 1H), 5.10-5.15 (m, 1H), 6.38 (s, 1H), 6.88 (d, J=9.20 Hz, 1H), 7.51-7.57 (m, 1H), 7.68-7.72 (m, 1H), 7.72-7.76 (m, 1H), 8.13-8.19 (m, 2H), 8.48 (s, 1H), 8.79-8.81 (m, 1H), 10.29 (br s, 1H), 11.00 (br s, 1H), 11.34 (br s, 1H). Mass spec: m/z: 686.0 [M+H]⁺.

### Example 7 was Synthesised Following Scheme 10

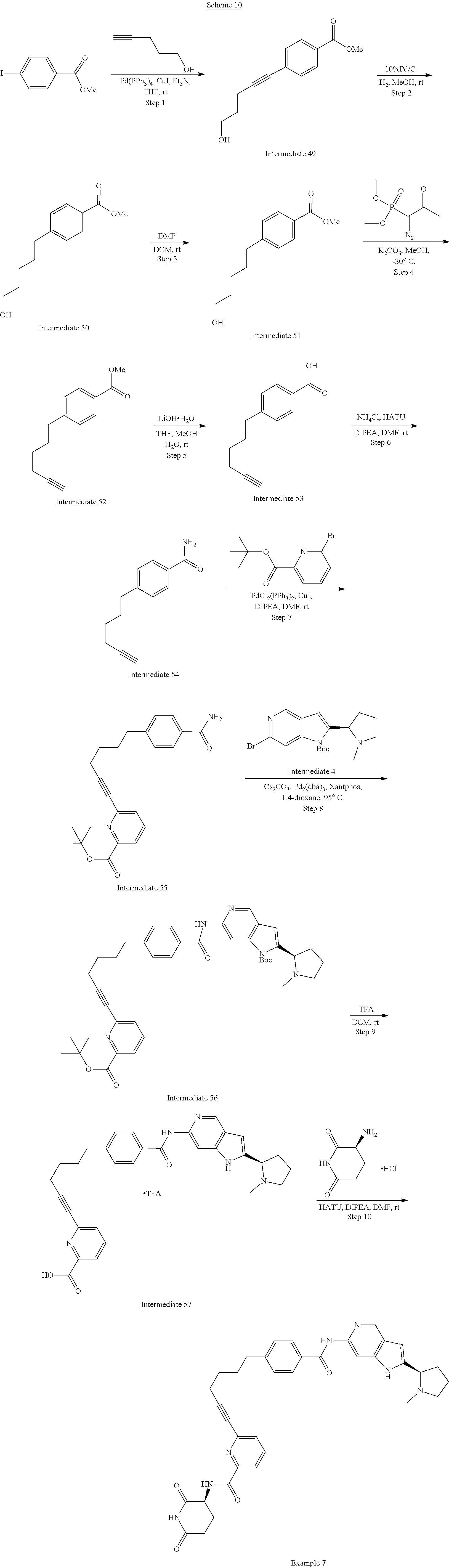

### Step 1

### Intermediate 49: methyl 4-(5-hydroxypent-1-yn-1-yl)benzoate

To a solution of methyl 4-iodobenzoate (CAS No: 619-44-3, 25.0 g, 95.4 mmol) in tetrahydrofuran (200 mL) was added pent-4-yn-1-ol (CAS No: 5390-04-5, 12.0 g, 143 mmol) followed by triethylamine (200 mL, 1430 mmol) and the reaction mixture was purged with argon gas for 15 min. Pd(PPh₃)₄ (5.51 g, 4.77 mmol) and copper(I) iodide (1.85 g, 9.54 mmol) were added and the reaction mixture was further purged with argon gas for 10 min and then stirred at room temperature for 16 h. The reaction mixture was diluted with water (400 mL) and extracted with ethyl acetate (3×500 mL). The combined organic phases were dried over anhydrous Na₂SO₄, filtered, and concentrated in vacuo. The crude material was purified by combi-flash column chromatography by eluting with 80% ethyl acetate in heptane to afford methyl 4-(5-hydroxypent-1-yn-1-yl)benzoate (Intermediate 49, 16.0 g, 77%) as a yellow solid. ¹H NMR (400 MHz, DMSO-d₆) δ ppm 1.67-1.73 (m, 2H), 2.50 (t, J=6.40 Hz, 2H), 3.50-3.55 (m, 2H), 3.84 (s, 3H), 4.55 (t, J=5.20 Hz, 1H), 7.49 (d, J=8.30 Hz, 2H), 7.89 (d, J=8.40 Hz, 2H). Mass spec: m/z: 219.4 [M+H]⁺.

### Step 2

### Intermediate 50: methyl 4-(5-hydroxypentyl)benzoate

To a solution of methyl 4-(5-hydroxypent-1-yn-1-yl)benzoate (Intermediate 49, 16.0 g, 73.3 mmol) in methanol (200 mL) was added 10% Pd/C (5.86 g, 55.0 mmol) and the reaction mixture was stirred at room temperature for 16 h under H₂ (110 psi). The reaction mixture was filtered through celite, washed with methanol (500 mL) and the filtrate was concentrated in vacuo to afford methyl 4-(5-hydroxypentyl)benzoate (Intermediate 50, 14.0 g, 86%) as a yellow solid, which was used for the next step without further purification. ¹H NMR (400 MHz, DMSO-d₆) δ ppm 1.24-1.35 (m, 2H), 1.39-1.48 (m, 2H), 1.54-1.62 (m, 2H), 2.64 (t, J=7.60 Hz, 2H), 3.34-3.41 (m, 2H), 3.83 (s, 3H), 4.33 (t, J=5.20 Hz, 1H), 7.33 (d, J=8.40 Hz, 2H), 7.86 (d, J=8.40 Hz, 2H). Mass spec: m/z: 223.16 [M+H]⁺.

### Step 3

### Intermediate 51: methyl 4-(5-oxopentyl)benzoate

To a solution of methyl 4-(5-hydroxypentyl)benzoate (Intermediate 50, 14.0 g, 62.98 mmol) in dichloromethane (200 mL) was added Dess-Martin periodinane (40.07 g, 94.47 mmol) at 0° C. The reaction mixture was then stirred at room temperature for 2 h. The reaction mixture was filtered through celite, filtrates were quenched with saturated NaHCO₃ solution (200 mL) and extracted with ethyl acetate (3×200 mL). The combined organic phases were dried over anhydrous Na₂SO₄, filtered and concentrated in vacuo. The crude material was purified by combi-flash chromatography by eluting with 30% ethyl acetate in heptane to afford methyl 4-(5-oxopentyl)benzoate (Intermediate 51, 8.00 g, 58%) as yellow solid. ¹H NMR (400 MHz, DMSO-d₆) δ ppm 1.47-1.64 (m, 4H), 2.21-2.25 (m, 1H), 2.43-2.48 (m, 1H), 2.61-2.69 (m, 2H), 3.83 (s, 3H), 7.34 (d, J=8.20 Hz, 2H), 7.87 (d, J=8.20 Hz, 2H), 9.65 (s, 1H).

### Step 4

### Intermediate 52: methyl 4-(hex-5-yn-1-yl)benzoate

To a solution of methyl 4-(5-oxopentyl)benzoate (Intermediate 51, 8.00 g, 36.3 mmol) in methanol (200 mL) was added potassium carbonate (6.01 g, 43.6 mmol) at 0° C. Dimethyl (1-diazo-2-oxopropyl)phosphonate (9.08 mL, 54.5 mmol) was added dropwise to the reaction mixture, at 0° C., over 20 min and the reaction mixture was stirred at −30° C. for 3 h. The reaction mixture was diluted with water (50 mL) and extracted with ethyl acetate (3×50 mL). The combined organic phases were dried over anhydrous Na₂SO₄ and concentrated in vacuo to obtain crude compound. The crude material was purified by combi-flash chromatography by eluting with 10% ethyl acetate in heptane to afford methyl 4-(hex-5-yn-1-yl)benzoate (Intermediate 52, 4.00 g, 51%) as yellow liquid. ¹H NMR (400 MHz, DMSO-d₆) δ ppm 1.40-1.50 (m, 2H), 1.60-1.72 (m, 2H), 2.17 (t, J=6.40 Hz, 2H), 2.66 (t, J=6.40 Hz, 2H), 2.73 (s, 1H), 3.83 (s, 3H), 7.34 (d, J=8.0 Hz, 2H), 7.87 (d, J=8.0 Hz, 2H).

### Step 5

### Intermediate 53: 4-(hex-5-yn-1-yl)benzoic acid

To a solution of methyl 4-(hex-5-yn-1-yl)benzoate (Intermediate 52, 4.00 g, 18.5 mmol) in tetrahydrofuran (50 mL), methanol (100 mL) and water (50 mL) was added lithium hydroxide (0.90 g, 37 mmol) and the reaction mixture was stirred at room temperature for 5 h. The reaction mixture was concentrated in vacuo and diluted with water (50 mL) and the aqueous phase was extracted with ethyl acetate (1×100 mL). The aqueous layer was then acidified up to pH~4 with 1N HCl and extracted with ethyl acetate (3×300 mL). The combined organic phases were dried over Na₂SO₄, and concentrated in vacuo to afford 4-(hex-5-yn-1-yl)benzoic acid (Intermediate 53, 3.3 g) as a white solid, which was used for the next step without further purification. ¹H NMR (400 MHz, DMSO-d₆) δ ppm 1.42-1.50 (m, 2H), 1.63-1.72 (m, 2H), 2.12-2.23 (m, 2H), 2.65 (t, J=7.60 Hz, 2H), 2.74 (s, 1H), 7.31 (d, J=8.0 Hz, 2H), 7.85 (d, J=8.0 Hz, 2H), 12.77 (br s, 1H). Mass spec: m/z: 201.5 [M−H]⁻.

### Step 6

### Intermediate 54: 4-(hex-5-yn-1-yl)benzamide

To a solution of 4-(hex-5-yn-1-yl)benzoic acid (Intermediate 53, 3.3 g, 16 mmol) in dimethylformamide (60 mL) was added HATU (9.6 g, 24 mmol) and N,N-diisopropylethylamine (8.5 mL, 49 mmol) and the reaction was stirred at room temperature for 10 min. Ammonium chloride (4.4 g, 82 mmol) was added at 0° C. and the reaction mixture was stirred at room temperature for 16 h. The reaction mixture was diluted with ice cold water (200 mL), resulting in a precipitate which was filtered, washed with water (100 mL) and dried under vacuum to afford 4-(hex-5-yn-1-yl)benzamide (Intermediate 54, 2.70 g, 82%) as a white solid, which was used for next step without further purification. ¹H NMR (400 MHz, DMSO-d₆) δ ppm 1.41-1.50 (m, 2H), 1.61-1.70 (m, 2H), 2.13-2.22 (m, 2H), 2.63 (d, J=7.60 Hz, 2H), 2.74 (s, 1H), 7.24 (br s, 1H), 7.26 (d, J=7.60 Hz, 2H), 7.78 (d, J=7.60 Hz, 2H), 7.88 (br s, 1H). Mass spec: m/z: 202.5 [M+H]⁺.

### Step 7

### Intermediate 55: tert-butyl 6-(6-(4-carbamoylphenyl) hex-1-yn-1-yl)picolinate

To a solution of 4-(hex-5-yn-1-yl)benzamide (Intermediate 54, 0.25 g, 1.24 mmol) in dimethylformamide (3 mL) was added tert-butyl 6-bromopicolinate (CAS No: 910044-07-4, 0.42 g, 1.61 mmol) followed by N,N-diisopropylethylamine (0.7 mL, 3.73 mmol). The reaction mixture was purged with argon for 15 min followed by the addition of copper(I) iodide (0.025 g, 0.12 mmol) and PdCl₂(PPh₃)₂ (0.14 g, 0.18 mmol). The reaction mixture was further purged with argon for 10 min and then stirred at room temperature for 1 h. The reaction mixture was quenched with ice cold water (50 mL) and extracted with ethyl acetate (2×100 mL). The combined organic phases were dried over anhydrous Na₂SO₄ and concentrated in vacuo. The crude material was purified by combi-flash chromatography by eluting with 70% ethyl acetate in heptane to afford tert-butyl 6-(6-(4-carbamoylphenyl)hex-1-yn-1-yl)picolinate (Intermediate 55, 0.2 g, 42%) as a yellow solid. ¹H NMR (400 MHz, DMSO-d₆) δ ppm 1.53 (s, 9H), 1.68-1.77 (m, 2H), 2.64-2.73 (m, 4H), 2.84-2.88 (m, 2H), 7.24 (br s, 1H), 7.26-7.28 (m, 2H), 7.61 (d, J=7.60 Hz, 1H), 7.77 (d, J=7.60 Hz, 2H), 7.86 (br s, 1H), 7.88-7.93 (m, 2H). Mass spec: m/z: 379.3 [M+H]⁺.

### Step 8

### Intermediate 56: tert-butyl (R)-6-(4-(6-(6-(tert-butoxycarbonyl)pyridin-2-yl)hex-5-yn-1-yl)benzamido)-2-(1-methylpyrrolidin-2-yl)-1H-pyrrolo[3,2-c]pyridine-1-carboxylate

To a solution of tert-butyl 6-(6-(4-carbamoylphenyl)hex-1-yn-1-yl) picolinate (Intermediate 55, 0.16 g, 0.42 mmol) in 1,4-dioxane (3 mL) was added tert-butyl (R)-6-bromo-2-(1-methylpyrrolidin-2-yl)-1H-pyrrolo[3,2-c]pyridine-1-carboxylate (Intermediate 4, 0.2 g, 0.52 mmol) followed by cesium carbonate (0.54 g, 1.57 mmol) at room temperature. The reaction mixture was purged with argon for 15 min followed by the addition of Pd₂(dba)₃ (0.067 g, 0.078 mmol) and Xantphos (0.096 g, 0.16 mmol). The reaction mixture was further purged with argon for 10 min and then heated at 95° C. for 5 h. The reaction mixture was concentrated in vacuo to obtain crude compound, which was purified by combi-flash chromatography by eluting with 7% MeOH in DCM to afford tert-butyl (R)-6-(4-(6-(6-(tert-butoxycarbonyl)pyridin-2-yl)hex-5-yn-1-yl)benzamido)-2-(1-methylpyrrolidin-2-yl)-1H-pyrrolo[3,2-c]pyridine-1-carboxylate (Intermediate 56, 0.2 g, 34%) as a yellow solid. ¹H NMR (400 MHz, DMSO-d₆) δ ppm 1.55 (s, 9H), 1.57-1.64 (m, 4H), 1.69 (s, 9H), 1.74-1.80 (m, 4H), 2.27-2.33 (m, 1H), 2.35 (s, 3H), 2.38-2.40 (m, 1H), 2.65-2.77 (m, 3H), 3.12-3.16 (m, 1H), 3.87-3.97 (m, 1H), 6.74 (s, 1H), 7.34-7.36 (m, 1H), 7.59-7.67 (m, 1H), 7.77-7.80 (m, 1H), 7.88-8.01 (m, 4H), 8.58 (s, 1H), 8.92 (s, 1H), 10.59 (br s, 1H). Mass spec: m/z: 678.4 [M+H]⁺.

### Step 9

### Intermediate 57: (R)-6-(6-(4-((2-(1-methylpyrrolidin-2-yl)-1H-pyrrolo[3,2-c]pyridin-6-yl)carbamoyl)phenyl) hex-1-yn-1-yl)picolinic acid trifluoroacetate

To a solution of tert-butyl (R)-6-(4-(6-(6-(tert-butoxycarbonyl)pyridin-2-yl)hex-5-yn-1-yl)benzamido)-2-(1-methylpyrrolidin-2-yl)-1H-pyrrolo[3,2-c]pyridine-1-carboxylate (Intermediate 56, 0.2 g, 0.29 mmol) in dichloromethane (2 mL) was added trifluoroacetic acid (0.57 mL, 7.37 mmol) at 0° C. The reaction mixture was then stirred at room temperature for 16 h. The reaction mixture was concentrated in vacuo to obtain crude compound, which was washed with diethyl ether (5×2 mL) and dried in vacuo to afford (R)-6-(6-(4-((2-(1-methylpyrrolidin-2-yl)-1H-pyrrolo[3,2-c]pyridin-6-yl)carbamoyl)phenyl)hex-1-yn-1-yl)picolinic acid trifluoroacetate (Intermediate 57, 0.2 g, 65%) as a red gum, which was used in the next step without further purification. Mass spec: m/z: 522.1 [M+H]⁺.

### Step 10

### Example 7: N—((S)-2,6-dioxopiperidin-3-yl)-6-(6-(4-((2-((R)-1-methylpyrrolidin-2-yl)-1H-pyrrolo[3,2-c]pyridin-6-yl)carbamoyl)phenyl)hex-1-yn-1-yl)picolinamide

To a solution of (R)-6-(6-(4-((2-(1-methylpyrrolidin-2-yl)-1H-pyrrolo[3,2-c]pyridin-6-yl) carbamoyl)phenyl) hex-1-yn-1-yl) picolinic acid trifluoroacetate (Intermediate 57, 0.19 g, 0.29 mmol) in dimethylformamide (3 mL) was added HATU (0.23 g, 0.59 mmol) and N,N-diisopropylethylamine (0.2 mL, 0.89 mmol) at 0° C. and the reaction stirred for 10 min. (S)-3-aminopiperidine-2,6-dione hydrochloride (CAS No: 25181-50-4, 0.074 g, 0.45 mmol) was added at 0° C. and the reaction mixture was stirred at room temperature for 3 h. The reaction mixture was poured into ice cold water (50 mL), resulting in a precipitate which was filtered and dried under vacuum to obtain the crude compound. The crude material was purified by preparative HPLC (Method A) to afford N—((S)-2,6-dioxopiperidin-3-yl)-6-(6-(4-((2-((R)-1-methylpyrrolidin-2-yl)-1H-pyrrolo[3,2-c]pyridin-6-yl)carbamoyl)phenyl)hex-1-yn-1-yl)picolinamide (Example 7, 0.025 g, 13% d) as an off white solid. ¹H NMR (400 MHz, DMSO-d₆) δ ppm 1.58-1.66 (m, 2H), 1.75-2.03 (m, 6H), 2.11-2.15 (m, 1H), 2.17 (s, 3H), 2.21-2.30 (m, 2H), 2.53-2.57 (m, 3H), 2.69-2.85 (m, 3H), 3.08-3.19 (m, 1H), 3.28-3.30 (m, 1H), 4.75-4.85 (m, 1H), 6.39 (s, 1H), 7.34-7.36 (m, 2H), 7.66-7.70 (m, 1H), 7.95-8.01 (m, 4H), 8.19 (s, 1H), 8.50 (s, 1H), 8.94-8.98 (m, 1H), 10.33 (br s, 1H), 10.89 (br s, 1H), 11.35 (br s, 1H). Mass spec: m/z: 632.1 [M+H]⁺.

### Example 8 was Synthesised Following Scheme 11

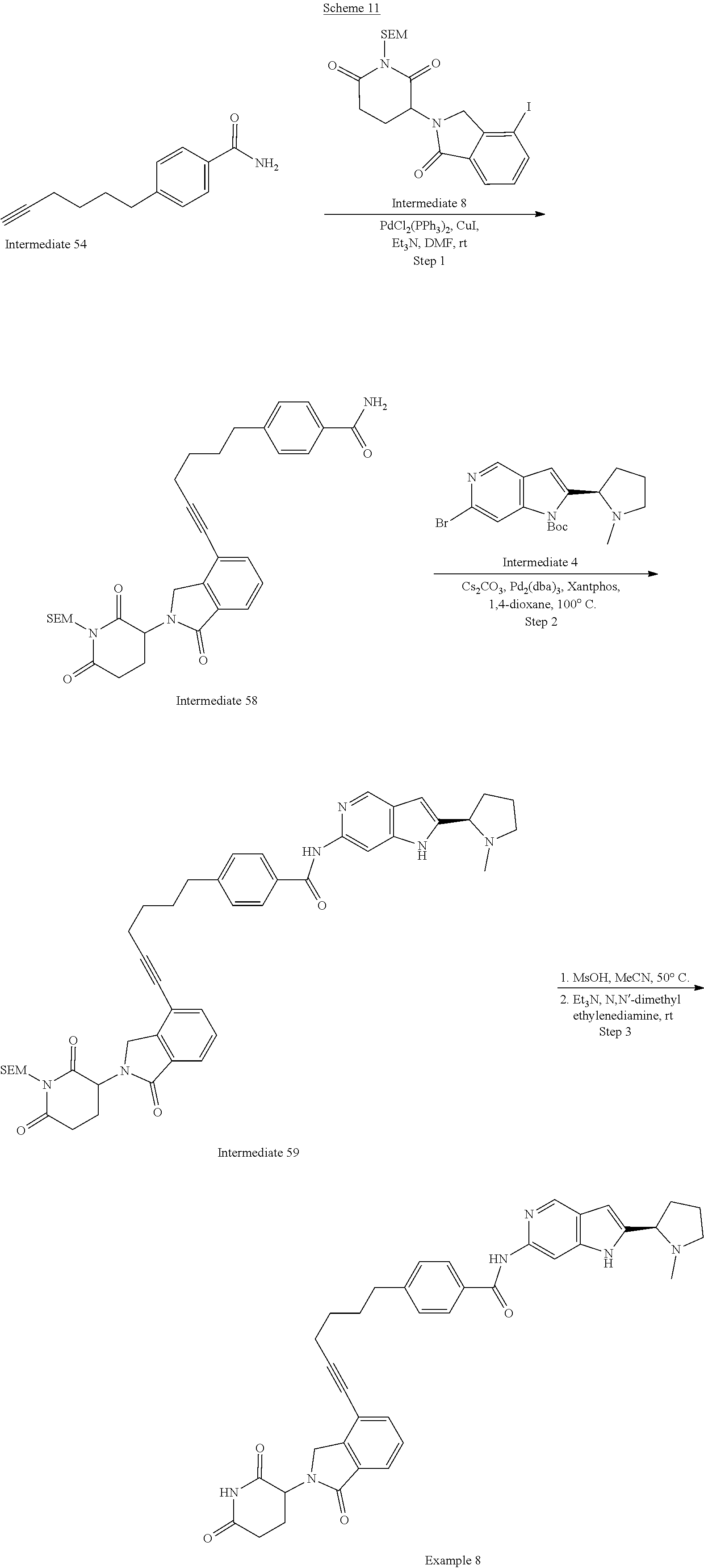 [SMILES: C#CCCCCc1ccc(C(N)=O)cc1 ; C[Si](C)(C)CCOCN1C(=O)CCC(N2Cc3c(I)cccc3C2=O)C1=O ; C[Si](C)(C)CCOCN1C(=O)CCC(N2Cc3c(C#CCCCCc4ccc(C(N)=O)cc4)cccc3C2=O)C1=O ; CN1CCC[C@@H]1c1cc2cnc(Br)cc2n1C(=O)OC(C)(C)C ; CN1CCC[C@@H]1c1cc2cnc(NC(=O)c3ccc(CCCCC#Cc4cccc5c4CN(C4CCC(=O)N(COCC[Si](C)(C)C)C4=O)C5=O)cc3)cc2[nH]1 ; CN1CCC[C@@H]1c1cc2cnc(NC(=O)c3ccc(CCCCC#Cc4cccc5c4CN(C4CCC(=O)NC4=O)C5=O)cc3)cc2[nH]1]

### Step 1

### Intermediate 58: 4-(6-(2-(2,6-dioxo-1-((2-(trimethylsilyl) ethoxy)methyl) piperidin-3-yl)-1-oxoisoindolin-4-yl) hex-5-yn-1-yl)benzamide

To a solution of 4-(hex-5-yn-1-yl)benzamide (Intermediate 54, 0.60 g, 2.98 mmol) in dimethylformamide (2 mL) was added 3-(4-iodo-1-oxoisoindolin-2-yl)-1-((2-(trimethylsilyl)ethoxy)methyl)piperidine-2,6-dione (Intermediate 8, 1.49 g, 2.98 mmol) followed by triethylamine (14.6 mL, 104.3 mmol). The reaction mixture was purged with argon for 15 min, then copper (I) iodide (0.057 g, 0.29 mmol) and PdCl₂(PPh₃)₂ (0.22 g, 0.29 mmol) were added. The reaction mixture was further purged with argon for 10 min and stirred at room temperature for 2 h. The reaction mixture was then diluted with water (100 mL) and extracted with ethyl acetate (3×100 mL). The combined organic layers were dried over anhydrous Na₂SO₄ and concentrated in vacuo. The crude material was purified by combi-flash chromatography, by eluting with 90% ethyl acetate in heptane, to afford 4-(6-(2-(2,6-dioxo-1-((2-(trimethylsilyl)ethoxy)methyl)piperidin-3-yl)-1-oxoisoindolin-4-yl)hex-5-yn-1-yl)benzamide (Intermediate 58, 1.00 g, 58%) as an off-white solid. ¹H NMR (400 MHz, DMSO-d₆) δ ppm −0.06 (s, 9H), 0.76-0.87 (m, 2H), 1.52-1.62 (m, 2H), 1.68-1.78 (m, 2H), 1.99-2.08 (m, 1H), 2.33-2.38 (m, 1H), 2.42-2.46 (m, 2H), 2.64-2.68 (m, 2H), 2.75-2.80 (m, 1H), 2.99-3.10 (m, 1H), 3.46-3.54 (m, 2H), 4.20-4.26 (m, 1H), 4.40-4.44 (m, 1H), 4.98-5.08 (m, 2H), 5.21-5.26 (m, 1H), 7.22 (br s, 1H), 7.25 (d, J=8.0 Hz, 2H), 7.50 (t, J=7.60 Hz, 1H), 7.62 (d, J=7.60 Hz, 1H), 7.69 (d, J=7.60 Hz, 1H), 7.76 (d, J=8.0 Hz, 2H), 7.84 (br s, 1H). Mass spec m/z 572.52 [M+H]⁺.

### Step 2

### Intermediate 59: tert-butyl-6-(4-(6-(2-(2,6-dioxo-1-((2-(trimethylsilyl)ethoxy)methyl)piperidin-3-yl)-1-oxoisoindolin-4-yl)hex-5-yn-1-yl)benzamido)-2-((R)-1-methylpyrrolidin-2-yl)-1H-pyrrolo[3,2-c]pyridine-1-carboxylate

To a solution of 4-(6-(2-(2,6-dioxo-1-((2-(trimethylsilyl)ethoxy)methyl)piperidin-3-yl)-1-oxoisoindolin-4-yl)hex-5-yn-1-yl)benzamide (Intermediate 58, 0.40 g, 0.69 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.26 g, 0.69 mmol) followed by cesium carbonate (0.46 g, 1.39 mmol). The reaction mixture was purged with argon for 15 min, then Pd₂(dba)₃ (0.098 g, 0.10 mmol) and Xantphos (0.12 g, 0.21 mmol) were added. The reaction mixture was further purged with argon for 10 min and then heated at 100° C. for 2 h. The reaction mixture was concentrated in vacuo and the crude material was purified by combi-flash chromatography, by eluting with 90% ethyl acetate in heptane, to afford tert-butyl-6-(4-(6-(2-(2,6-dioxo-1-((2-(trimethylsilyl)ethoxy)methyl)piperidin-3-yl)-1-oxoisoindolin-4-yl)hex-5-yn-1-yl)benzamido)-2-((R)-1-methylpyrrolidin-2-yl)-1H-pyrrolo[3,2-c]pyridine-1-carboxylate (Intermediate 59, 0.45 g, 74%) as a white solid. ¹H NMR (400 MHz, DMSO-d₆) δ ppm −0.05 (s, 9H), 0.76-0.86 (m, 2H), 1.58-1.66 (m, 4H), 1.69 (s, 9H), 1.73-1.82 (m, 4H), 2.02-2.10 (m, 1H), 2.28-2.33 (m, 2H), 2.35 (s, 3H), 2.37-2.42 (m, 1H), 2.66-2.78 (m, 4H), 3.01-3.17 (m, 2H), 3.47-3.54 (m, 2H), 3.88-3.94 (m, 1H), 4.24-4.28 (m, 1H), 4.43-4.50 (m, 1H), 4.98-5.08 (m, 2H), 5.24-5.30 (m, 1H), 6.75 (s, 1H), 7.34 (d, J=8.0 Hz, 2H), 7.53 (t, J=7.20 Hz, 1H), 7.65 (d, J=8.0 Hz, 1H), 7.72 (d, J=7.20 Hz, 1H), 7.97 (d, J=7.20 Hz, 2H), 8.58 (s, 1H), 8.93 (s, 1H), 10.60 (s, 1H). Mass spec m/z 873.57 [M+H]⁺.

### Step 3

### Example 8: 4-(6-(2-(2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide

To a solution of tert-butyl-6-(4-(6-(2-(2,6-dioxo-1-((2-(trimethylsilyl)ethoxy)methyl)piperidin-3-yl)-1-oxoisoindolin-4-yl)hex-5-yn-1-yl)benzamido)-2-((R)-1-methylpyrrolidin-2-yl)-1H-pyrrolo[3,2-c]pyridine-1-carboxylate (Intermediate 59, 0.40 g, 0.54 mmol) in acetonitrile (5 mL) was added methanesulfonic acid (0.53 mL, 8.07 mmol) at room temperature and the reaction mixture was heated at 50° C. for 2 h. After cooling to room temperature, N,N′-dimethylethylenediamine (0.39 mL, 3.23 mmol) followed by triethylamine (1.10 g, 10.77 mmol were added the reaction mixture was stirred at room temperature for 3 h. The reaction mixture was diluted with water (100 mL), the resultant precipitate was collected by filtration and dried in vacuo. The crude material was purified by preparative HPLC (Method A) to afford 4-(6-(2-(2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide (Example 8, 0.15 g, 44%) as an off white solid. ¹H NMR (400 MHz, DMSO-d₆) δ ppm 1.58-1.66 (m, 2H), 1.74-1.95 (m, 5H), 1.97-2.05 (m, 1H), 2.10-2.15 (m, 1H), 2.17 (s, 3H), 2.23-2.30 (m, 1H), 2.40-2.45 (m, 1H), 2.53-2.58 (m, 2H), 2.59-2.64 (m, 1H), 2.70-2.76 (m, 2H), 2.85-2.96 (m, 1H), 3.10-3.17 (m, 1H), 3.32-3.36 (m, 1H), 4.28-4.36 (m, 1H), 4.40-4.48 (m, 1H), 5.11-5.16 (m, 1H), 6.39 (s, 1H), 7.34 (d, J=8.0 Hz, 2H), 7.52 (t, J=8.0 Hz, 1H), 7.63 (d, J=7.60 Hz, 1H), 7.70 (d, J=7.60 Hz, 1H), 7.98 (d, J=8.0 Hz, 2H), 8.18 (s, 1H), 8.49 (s, 1H), 10.33 (s, 1H), 11.01 (br s, 1H), 11.35 (s, 1H). Mass spec m/z 643.5 [M+H]⁺.

### Example 9 was Synthesised Following Scheme 12

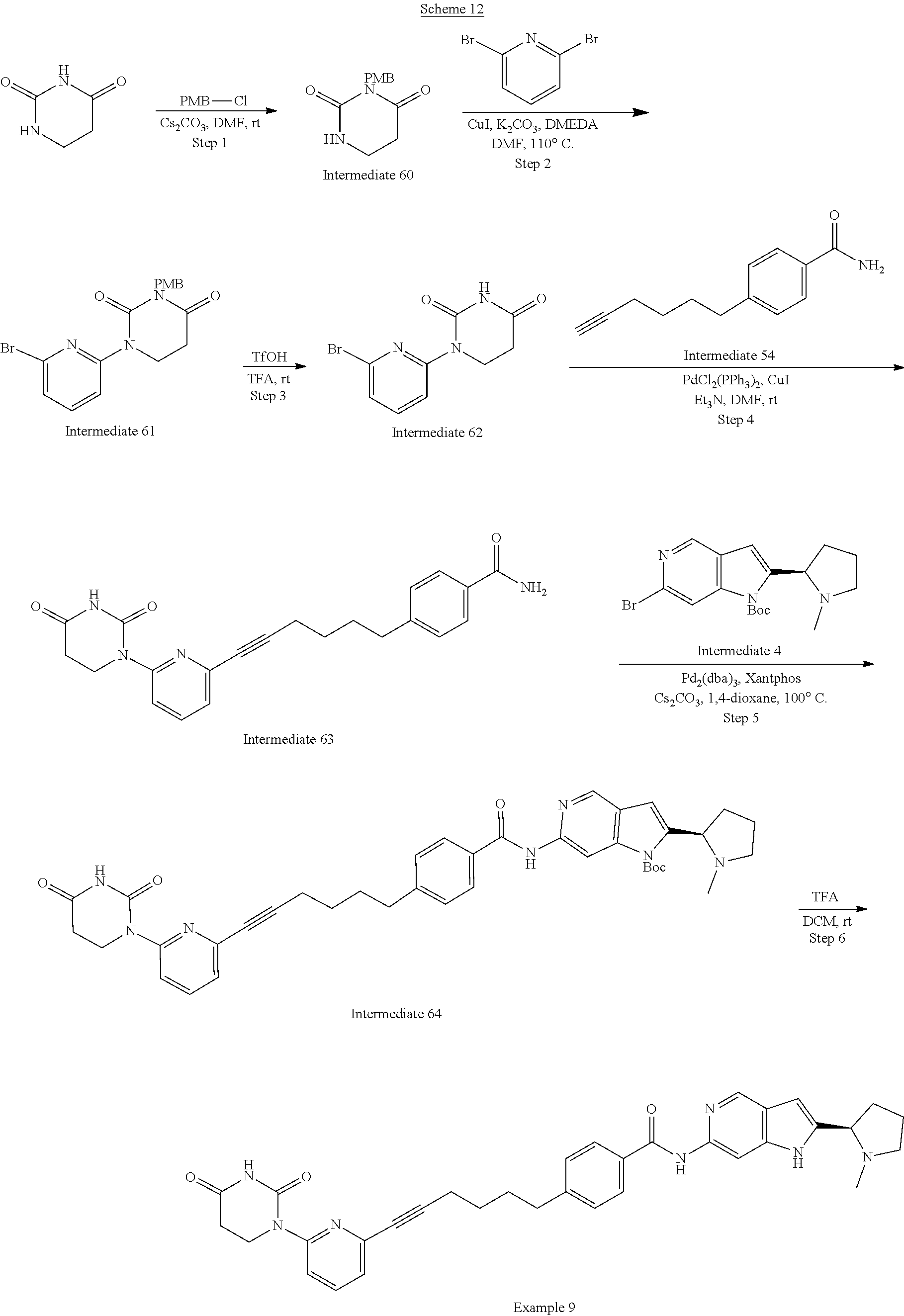

### Step 1

### Intermediate 60: 3-(4-methoxybenzyl) dihydropyrimidine-2,4(1H, 3H)-dione

To a solution of dihydropyrimidine-2,4(1H,3H)-dione (CAS No: 504-07-4, 5.00 g, 43.82 mmol) in dimethylformamide (150 mL) was added cesium carbonate (28.56 g, 87.64 mmol) followed by 4-methoxybenzyl chloride (4.06 mL, 28.48 mmol) and the reaction mixture was stirred at room temperature for 16 h. The reaction mixture was quenched with ice cold water (1500 mL) and stirred for 30 min, resulting in a precipitate, which was filtered and dried in vacuo to afford 3-(4-methoxybenzyl)dihydropyrimidine-2,4(1H,3H)-dione (Intermediate 60, 4.50 g, 44%) as an off white solid, which was used for the next step without further purification. ¹H NMR (400 MHz, DMSO-d₆) δ ppm 2.62 (t, J=6.80 Hz, 2H), 3.19-3.23 (m, 2H), 3.71 (s, 3H), 4.71 (s, 2H), 6.83-6.85 (m, 2H), 7.16-7.18 (m, 2H), 7.80 (br s, 1H).

### Step 2

### Intermediate 61: 1-(6-bromopyridin-2-yl)-3-(4-methoxybenzyl) dihydropyrimidine-2,4(1H, 3H)-dione

To a solution of 2,6-dibromopyridine (CAS No: 626-05-1, 7.00 g, 29.5 mmol) in dimethylformamide (10 mL) was added 3-(4-methoxybenzyl)dihydropyrimidine-2,4(1H, 3H)-dione (Intermediate 60, 4.15 g, 17.7 mmol) followed by K₂CO₃ (12.3 g, 88.6 mmol). The reaction mixture was purged with argon gas for 15 min then copper(I) iodide (0.56 g, 2.95 mmol) and N,N-dimethylethylenediamine (0.53 g, 5.91 mmol) were added and the reaction mixture was heated at 110° C. for 16 h. The reaction mixture was filtered and washed with ethyl acetate (100 mL). the organic phases was washed with ice cold water (100 mL), dried over Na₂SO₄, and concentrated in vacuo. The crude material was purified by combi-flash column chromatography by eluting with 20% ethyl acetate in heptane to afford 1-(6-bromopyridin-2-yl)-3-(4-methoxybenzyl)dihydropyrimidine-2,4(1H,3H)-dione (Intermediate 61, 3.4 g, 29%) as an off white solid. ¹H NMR (400 MHz, DMSO-d₆) δ ppm 2.88 (t, J=6.80 Hz, 2H), 3.72 (s, 3H), 4.02 (t, J=6.80 Hz, 2H), 4.83 (s, 2H), 6.84-6.86 (m, 2H), 7.22-7.25 (m, 2H), 7.42-7.47 (m, 1H), 7.76-7.79 (m, 2H).

### Step 3

### Intermediate 62: 1-(6-bromopyridin-2-yl) dihydropyrimidine-2,4(1H, 3H)-dione

To a solution of 1-(6-bromopyridin-2-yl)-3-(4-methoxybenzyl)dihydropyrimidine-2,4(1H, 3H)-dione (Intermediate 61, 4.00 g, 10.3 mmol) in trifluoroacetic acid (40 mL) was added trifluoromethanesulfonic acid (10.00 mL) and the reaction was stirred at room temperature for 16 h. The reaction mixture was concentrated at a lower temperature. The obtained residue was quenched with saturated NaHCO₃, to pH~8, resulting in a precipitate which was collected by filtration, washed with saturated NaHCO₃ solution (50 mL) and water (50 mL) and dried in vacuo to afford 1-(6-bromopyridin-2-yl) dihydropyrimidine-2,4(1H, 3H)-dione (Intermediate 62, 3.50 g) as a brown solid, which was used for the next step without further purification. ¹H NMR (400 MHz, DMSO-d₆) δ ppm 2.66-2.70 (m, 2H), 4.01-4.04 (m, 2H), 7.40-7.44 (m, 1H), 7.72-7.79 (m, 1H), 7.80-7.86 (m, 1H), 10.63 (br s, 1H). Mass spec: m/z: 270.2 [M+H]⁺.

### Step 4

### Intermediate 63: 4-(6-(6-(2,4-dioxotetrahydropyrimidin-1(2H)-yl) pyridin-2-yl)hex-5-yn-1-yl)benzamide

To a solution of 4-(hex-5-yn-1-yl)benzamide (Intermediate 54, 0.65 g, 3.23 mmol) in dimethylformamide (15 mL) was added 1-(6-bromopyridin-2-yl)dihydropyrimidine-2,4(1H, 3H)-dione (Intermediate 62, 1.04 g, 3.87 mmol) followed by triethylamine (15.8 mL, 113.0 mmol). The reaction mixture was purged with argon for 15 min and then copper(I) iodide (0.064 g, 0.32 mmol) and PdCl₂(PPh₃)₂ (0.23 g, 0.32 mmol) were added. The reaction mixture was further purged with argon for 10 min and stirred at room temperature for 2 h. The reaction mixture was quenched with ice cold water (80 mL) and extracted with ethyl acetate (2×150 mL). The combined organic phases were dried over anhydrous Na₂SO₄ and concentrated in vacuo. The crude material was purified by combi-flash chromatography by eluting with 100% ethyl acetate to afford 4-(6-(6-(2,4-dioxotetrahydropyrimidin-1(2H)-yl) pyridin-2-yl) hex-5-yn-1-yl)benzamide (Intermediate 63, 0.80 g, 63%) as a yellow solid. ¹H NMR (400 MHz, DMSO-d₆) δ ppm 1.51-1.60 (m, 2H), 1.70-1.80 (m, 2H), 2.44-2.47 (m, 2H), 2.63-2.71 (m, 4H), 3.98-4.04 (m, 2H), 7.21-7.29 (m, 4H), 7.68-7.80 (m, 4H), 7.88 (br s, 1H), 10.54 (s, 1H). Mass spec: m/z: 391.2 [M+H]⁺.

### Step 5

### Intermediate 64: tert-butyl (R)-6-(4-(6-(6-(2,4-dioxotetrahydropyrimidin-1(2H)-yl)pyridin-2-yl)hex-5-yn-1-yl)benzamido)-2-(1-methylpyrrolidin-2-yl)-1H-pyrrolo[3,2-c]pyridine-1-carboxylate

To a solution of 4-(6-(6-(2,4-dioxotetrahydropyrimidin-1(2H)-yl) pyridin-2-yl) hex-5-yn-1-yl)benzamide (Intermediate 63, 0.50 g, 1.28 mmol) in 1,4-dioxane (20 mL) was added tert-butyl (R)-6-bromo-2-(1-methylpyrrolidin-2-yl)-1H-pyrrolo[3,2-c]pyridine-1-carboxylate (Intermediate 4, 0.58 g, 1.54 mmol) followed by cesium carbonate (1.25 g, 3.84 mmol) at room temperature. The reaction mixture was purged with argon for 15 min and then Pd₂(dba)₃ (0.18 g, 0.19 mmol) and Xantphos (0.23 g, 0.38 mmol) were added. The reaction mixture was further purged with argon for 10 min and stirred at 100° C. for 2 h. The reaction mixture was filtered through celite, washed with ethyl acetate (100 mL) and filtrate was concentrated in vacuo to obtain crude compound. The crude material was purified by combi-flash column chromatography by eluting with 5% MeOH in DCM to afford tert-butyl (R)-6-(4-(6-(6-(2,4-dioxotetrahydropyrimidin-1(2H)-yl)pyridin-2-yl)hex-5-yn-1-yl)benzamido)-2-(1-methylpyrrolidin-2-yl)-1H-pyrrolo[3,2-c]pyridine-1-carboxylate (Intermediate 64, 0.50 g, 57%) as a yellow solid. ¹H NMR (400 MHz, DMSO-d₆) δ ppm 1.55-1.66 (m, 4H), 1.69 (s, 9H), 1.72-1.81 (m, 4H), 2.29-2.32 (m, 1H), 2.35 (s, 3H), 2.37-2.42 (m, 2H), 2.64-2.77 (m, 4H), 3.07-3.18 (m, 1H), 3.88-3.95 (m, 1H), 3.98-4.06 (m, 2H), 6.75 (s, 1H), 7.22-7.26 (m, 1H), 7.35 (d, J=8.20 Hz, 2H), 7.67-7.79 (m, 2H), 7.98 (d, J=8.20 Hz, 2H), 8.59 (s, 1H), 8.92 (s, 1H), 10.55 (br s, 1H), 10.61 (br s, 1H). Mass spec: m/z: 690.0 [M+H]⁺.

### Step 6

### Example 9: (R)-4-(6-(6-(2,4-dioxotetrahydropyrimidin-1(2H)-yl)pyridin-2-yl)hex-5-yn-1-yl)-N-(2-(1-methylpyrrolidin-2-yl)-1H-pyrrolo[3,2-c]pyridin-6-yl)benzamide

To a solution of tert-butyl (R)-6-(4-(6-(6-(2,4-dioxotetrahydropyrimidin-1(2H)-yl) pyridin-2-yl)hex-5-yn-1-yl)benzamido)-2-(1-methylpyrrolidin-2-yl)-1H-pyrrolo[3,2-c]pyridine-1-carboxylate (Intermediate 64, 0.40 g, 0.58 mmol) in dichloromethane (10 mL) was added trifluoroacetic acid (5 mL) at 0° C. and the reaction mixture was stirred at room temperature for 1 h. The volatiles were removed under reduced pressure and co-distilled with DCM to obtain the crude compound which was purified by preparative HPLC (Method A) to afford (R)-4-(6-(6-(2,4-dioxotetrahydropyrimidin-1(2H)-yl)pyridin-2-yl)hex-5-yn-1-yl)-N-(2-(1-methylpyrrolidin-2-yl)-1H-pyrrolo[3,2-c]pyridin-6-yl)benzamide (Example 9, 0.15 g, 44%) as a yellow solid. ¹H NMR (400 MHz, DMSO-d₆) δ ppm 1.55-1.64 (m, 2H), 1.72-1.94 (m, 5H), 2.10-2.14 (m, 1H), 2.16 (s, 3H), 2.22-2.30 (m, 1H), 2.42-2.47 (m, 1H), 2.52-2.56 (m, 2H), 2.66-2.75 (m, 4H), 3.08-3.21 (m, 1H), 4.01-4.04 (m, 2H), 6.39 (s, 1H), 7.23-7.25 (m, 1H), 7.32-7.36 (m, 2H), 7.69-7.80 (m, 2H), 7.96-8.00 (m, 2H), 8.18 (s, 1H), 8.49 (s, 1H), 10.35 (br s, 1H), 10.55 (br s, 1H), 11.36 (br s, 1H). Mass spec: m/z: 590.2 [M+H]⁺.

### Example 10 was Synthesised Following Scheme 13

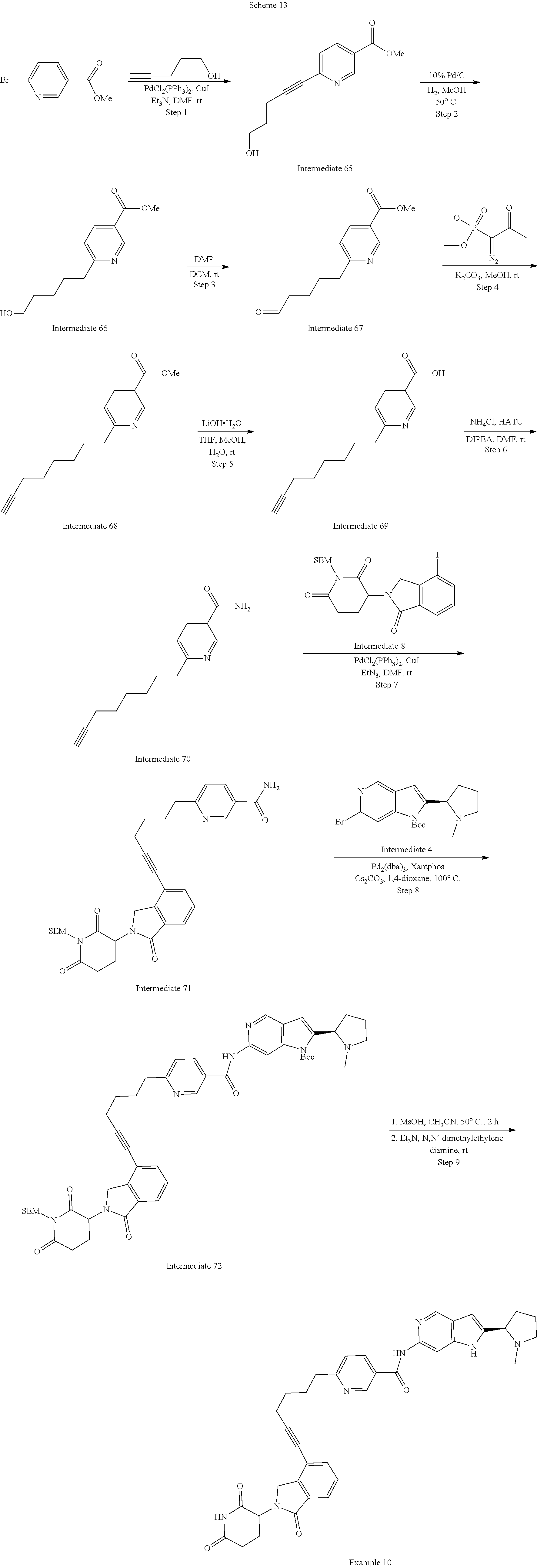

### Step 1

### Intermediate 65: methyl 6-(5-hydroxypent-1-yn-1-yl)nicotinate

To a solution of methyl 6-bromonicotinate (CAS No: 26218-78-0, 12.0 g, 54.4 mmol) in dimethylformamide (80 mL) was added pent-4-yn-1-ol (CAS No: 5390-04-5, 7.0 mL, 70.8 mmol) followed by triethylamine (229 mL, 1630 mmol). The reaction mixture was purged with argon gas for 15 min. PdCl₂(PPh₃)₂ (5.91 g, 8.17 mmol) and copper(I) iodide (1.64 g, 8.17 mmol) were then added and the reaction mixture was further purged with argon gas for 10 min and stirred at room temperature for 2 h. The reaction mixture was diluted with cold water (200 mL) and extracted with ethyl acetate (3×100 mL). The combined organic phases were washed with brine solution (2×100 mL), dried over anhydrous Na₂SO₄, filtered, and concentrated in vacuo. The crude material was purified by combi-flash chromatography by eluting with 60% ethyl acetate in heptane to afford methyl 6-(5-hydroxypent-1-yn-1-yl) nicotinate (Intermediate 65, 10.0 g, 84%) as a liquid. ¹H NMR (400 MHz, DMSO-d₆) δ ppm 1.68-1.76 (m, 2H), 2.54 (t, J=7.20 Hz, 2H), 3.43 (t, J=6.0 Hz, 1H), 3.51-3.55 (m, 2H), 3.88 (s, 3H), 7.58 (d, J=8.0 Hz, 1H), 8.23 (d, J=8.0 Hz, 1H), 9.00 (s, 1H). Mass spec: m/z: 220.4 [M+H]⁺.

### Step 2

### Intermediate 66: methyl 6-(5-hydroxypentyl)nicotinate

To a solution of methyl 6-(5-hydroxypent-1-yn-1-yl)nicotinate (Intermediate 65, 10.0 g, 45.61 mmol) in methanol (150 mL) was added 10% Pd/C (3.88 g, 36.49 mmol). The reaction mixture was heated at 50° C. for 5 h under an H₂ (140 psi). The reaction mixture was filtered through celite, washed with methanol (100 mL) and concentrated in vacuo to afford methyl 6-(5-hydroxypentyl)nicotinate (Intermediate 66, 8.00 g, 79%) as a yellow liquid, which was used for the next step without further purification. ¹H NMR (400 MHz, DMSO-d₆) δ ppm 1.25-1.33 (m, 2H), 1.38-1.46 (m, 2H), 1.63-1.70 (m, 2H), 2.78 (t, J=7.60 Hz, 2H), 3.32-3.38 (m, 2H), 3.85 (s, 3H), 4.31 (t, J=4.80 Hz, 1H), 7.40 (d, J=8.40 Hz, 1H), 8.17 (d, J=8.40 Hz, 1H), 8.97 (s, 1H).

### Step 3

### Intermediate 67: methyl 6-(5-oxopentyl)nicotinate

To a solution of methyl 6-(5-hydroxypentyl)nicotinate (Intermediate 66, 8.00 g, 36 mmol) in dichloromethane (100 mL) was added Dess-Martin periodinane (23.0 g, 54 mmol) at 0° C. The reaction mixture was then stirred at room temperature for 2 h. The reaction mixture was quenched with aqueous saturated NaHCO₃ solution (200 mL) followed by aqueous sodium thiosulfate (200 mL) solution and extracted with ethyl acetate (2×250 mL). The combined organic phases were dried over anhydrous Na₂SO₄, filtered, and concentrated in vacuo. The crude material was purified by combi-flash chromatography by eluting with 25% ethyl acetate in heptane to afford methyl 6-(5-oxopentyl) nicotinate (Intermediate 67, 4.00 g, 50%) as a yellow liquid. ¹H NMR (400 MHz, DMSO-d₆) δ ppm 1.48-1.56 (m, 2H), 1.64-1.72 (m, 2H), 2.41-2.46 (m, 2H), 2.80 (t, J=7.20 Hz, 2H), 3.85 (s, 3H), 7.41 (d, J=7.80 Hz, 1H), 8.17 (d, J=7.80 Hz, 1H), 8.97 (s, 1H), 9.64 (s, 1H). Mass spec: m/z: 222.5 [M+H]⁺.

### Step 4

### Intermediate 68: methyl 6-(hex-5-yn-1-yl)nicotinate

To a solution of methyl 6-(5-oxopentyl)nicotinate (Intermediate 67, 4.00 g, 18.1 mmol) in methanol (100 mL) was added potassium carbonate (2.99 g, 21.7 mmol) at 0° C., then dimethyl (1-diazo-2-oxopropyl) phosphonate (Comp-6, 4.52 mL, 27.1 mmol) was added dropwise to the reaction mixture for over 5 min. The reaction mixture was then stirred at room temperature for 16 h. The reaction mixture was concentrated and water (50 mL) was added and extracted with ethyl acetate (3×50 mL). The combined organic phases were dried over anhydrous Na₂SO₄, and concentrated in vacuo to obtain crude compound which was purified by combi-flash chromatography by eluting with 10% ethyl acetate in heptane to afford methyl 6-(hex-5-yn-1-yl) nicotinate (Intermediate 68, 2.90 g, 74%) as a colorless liquid. ¹H NMR (400 MHz, DMSO-d₆) δ ppm 1.42-1.52 (m, 2H), 1.71-1.82 (m, 2H), 2.15-2.23 (m, 2H), 2.74 (s, 1H), 2.82 (t, J=7.60 Hz, 2H), 3.87 (s, 3H), 7.42 (d, J=8.40 Hz, 1H), 8.19 (d, J=8.40 Hz, 1H), 8.99 (s, 1H). Mass spec: m/z: 218.1 [M+H]⁺.

### Step 5

### Intermediate 69: 6-(hex-5-yn-1-yl)nicotinic acid

To a solution of methyl 6-(hex-5-yn-1-yl)nicotinate (Intermediate 68, 2.80 g, 12.9 mmol) in tetrahydrofuran (12 mL), methanol (12 mL) and water (12 mL) was added lithium hydroxide (0.94 g, 38.7 mmol) and the reaction mixture was stirred at room temperature for 16 h. The reaction mixture was concentrated in vacuo. The crude residue was acidified by the addition of 1N HCl to pH~5 and then extracted with ethyl acetate (2×200 mL). The combined organic phases were dried over anhydrous Na₂SO₄ and concentrated in vacuo to afford 6-(hex-5-yn-1-yl)nicotinic acid (Intermediate 69, 2.2 g, 84%) as an off-white solid, which was used for the next step without further purification. ¹H NMR (400 MHz, DMSO-d₆) δ ppm 1.41-1.49 (m, 2H), 1.71-1.80 (m, 2H), 2.10-2.23 (m, 2H), 2.73 (s, 1H), 2.80 (t, J=7.60 Hz, 2H), 7.37 (d, J=8.00 Hz, 1H), 8.14 (d, J=8.00 Hz, 1H), 8.95 (s, 1H), 13.21 (br s, 1H). Mass spec: m/z: 204.5 [M+H]⁺.

### Step 6

### Intermediate 70: 6-(hex-5-yn-1-yl)nicotinamide

To a solution of 6-(hex-5-yn-1-yl)nicotinic acid (Intermediate 69, 2.00 g, 9.84 mmol) in dimethylformamide (20 mL) was added HATU (5.79 g, 14.8 mmol) and the reaction stirred at room temperature for 10 min. Ammonium chloride (2.64 g, 49.2 mmol) and N,N-diisopropylethylamine (5.15 mL, 29.5 mmol) were then added and the reaction mixture was stirred at room temperature for 16 h. The reaction mixture was poured into ice cold water (100 mL), resulting in a precipitate, which was collected by filtration and washed with water (2×50 mL) and dried under reduced pressure to afford 6-(hex-5-yn-1-yl)nicotinamide (Intermediate 70, 1.20 g, 60%) as an off-white solid, which was used for next step without further purification. ¹H NMR (400 MHz, DMSO-d₆) δ ppm 1.41-1.51 (m, 2H), 1.71-1.81 (m, 2H), 2.15-2.22 (m, 2H), 2.75 (s, 1H), 2.79 (t, J=7.60 Hz, 2H), 7.34 (d, J=8.0 Hz, 1H), 7.48 (br s, 1H), 8.06 (br s, 1H), 8.09 (d, J=8.0 Hz, 1H), 8.92 (s, 1H). Mass spec: m/z: 203.5 [M+H]⁺.

### Step 7

### Intermediate 71: 6-(6-(2-(2,6-dioxo-1-((2-(trimethylsilyl) ethoxy)methyl) piperidin-3-yl)-1-oxoisoindolin-4-yl) hex-5-yn-1-yl)nicotinamide

To a solution of 6-(hex-5-yn-1-yl) nicotinamide (Intermediate 70, 0.30 g, 1.48 mmol) in dimethylformamide (5 mL) was added 3-(4-iodo-1-oxoisoindolin-2-yl)-1-((2-(trimethylsilyl)ethoxy)methyl)piperidine-2,6-dione (Intermediate 8, 0.81 g, 1.63 mmol) followed by triethylamine (7.27 mL, 51.91 mmol). The reaction mixture was purged with argon for 15 min then copper (I) iodide (0.029 g, 0.15 mmol) and PdCl₂(PPh₃)₂ (0.11 g, 0.15 mmol) were added. The reaction mixture was further purged with argon for 10 min and stirred at room temperature for 2 h. The reaction mixture was filtered through celite, washed with ethyl acetate (100 mL) and the filtrate was concentrated in vacuo to obtain crude compound. The crude material was purified by combi-flash chromatography by eluting with 2% MeOH in DCM to afford 6-(6-(2-(2,6-dioxo-1-((2-(trimethylsilyl)ethoxy)methyl)piperidin-3-yl)-1-oxoisoindolin-4-yl)hex-5-yn-1-yl)nicotinamide (Intermediate 71, 0.45 g, 53%) as a yellow solid. ¹H NMR (400 MHz, DMSO-d₆) δ ppm −0.04 (s, 9H), 0.77-0.89 (m, 2H), 1.56-1.66 (m, 2H), 1.79-1.93 (m, 3H), 2.02-2.11 (m, 1H), 2.31-2.43 (m, 2H), 2.75-2.88 (m, 3H), 3.01-3.12 (m, 1H), 3.47-3.57 (m, 2H), 4.24-4.28 (m, 1H), 4.43-4.48 (m, 1H), 4.99-5.10 (m, 2H), 5.24-5.28 (m, 1H), 7.35 (d, J=8.0 Hz, 1H), 7.50 (br s, 1H), 7.52-7.54 (m, 1H), 7.64 (d, J=7.46 Hz, 1H), 7.72 (d, J=7.46 Hz, 1H), 8.05 (br s, 1H), 8.10 (d, J=8.0 Hz, 1H), 8.92 (br s, 1H). Mass spec: m/z: 575.1 [M+H]⁺.

### Step 8

### Intermediate 72: tert-butyl 6-(6-(6-(2-(2,6-dioxo-1-((2-(trimethylsilyl)ethoxy)methyl)piperidin-3-yl)-1-oxoisoindolin-4-yl)hex-5-yn-1-yl)nicotinamido)-2-((R)-1-methylpyrrolidin-2-yl)-1H-pyrrolo[3,2-c]pyridine-1-carboxylate

To a solution of 6-(6-(2-(2,6-dioxo-1-((2-(trimethylsilyl) ethoxy)methyl) piperidin-3-yl)-1-oxoisoindolin-4-yl) hex-5-yn-1-yl) nicotinamide (Intermediate 71, 0.41 g, 0.71 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 0.32 g, 0.85 mmol) followed by cesium carbonate (0.69 g, 2.14 mmol) at room temperature. The reaction mixture was purged with argon for 15 min then Pd₂(dba)₃ (0.10 g, 0.11 mmol) and Xantphos (0.13 g, 0.21 mmol) were added. The reaction mixture was further purged with argon for 10 min and then stirred at 100° C. for 2 h. The reaction mixture was filtered through celite, washed with ethyl acetate (100 mL) and the filtrate was concentrated in vacuo to obtain the crude compound. The crude material was purified by combi-flash chromatography by eluting with 5% MeOH in DCM to afford tert-butyl 6-(6-(6-(2-(2,6-dioxo-1-((2-(trimethylsilyl)ethoxy)methyl)piperidin-3-yl)-1-oxoisoindolin-4-yl)hex-5-yn-1-yl)nicotinamido)-2-((R)-1-methylpyrrolidin-2-yl)-1H-pyrrolo[3,2-c]pyridine-1-carboxylate (Intermediate 72, 0.31 g, 50%) as a yellow solid. ¹H NMR (400 MHz, DMSO-d₆) δ ppm −0.05 (s, 9H), 0.77-0.86 (m, 2H), 1.56-1.66 (m, 4H), 1.69 (s, 9H), 1.74-1.76 (m, 2H), 1.83-1.93 (m, 2H), 2.02-2.11 (m, 1H), 2.28-2.33 (m, 1H), 2.35 (s, 3H), 2.38-2.42 (m, 1H), 2.53-2.58 (m, 2H), 2.74-2.90 (m, 3H), 3.01-3.16 (m, 2H), 3.44-3.55 (m, 2H), 3.90-3.94 (m, 1H), 4.25-4.29 (m, 1H), 4.45-4.49 (m, 1H), 4.98-5.09 (m, 2H), 5.24-5.28 (m, 1H), 6.76 (s, 1H), 7.41 (d, J=8.0 Hz, 1H), 7.50-7.56 (m, 1H), 7.65 (d, J=7.60 Hz, 1H), 7.72 (d, J=7.60 Hz, 1H), 8.28 (d, J=80 Hz, 1H), 8.60 (s, 1H), 8.93 (s, 1H), 9.06 (s, 1H), 10.91 (br s, 1H). Mass spec: m/z: 873.8 [M+H]⁺.

### Step 9

### Example 10: 6-(6-(2-(2,6-dioxopiperidin-3-yl)-1-oxoisoindolin-4-yl) hex-5-yn-1-yl)-N-(2-((R)-1-methylpyrrolidin-2-yl)-1H-pyrrolo[3,2-c]pyridin-6-yl) nicotinamide

To a solution of tert-butyl 6-(6-(6-(2-(2,6-dioxo-1-((2-(trimethylsilyl)ethoxy)methyl)piperidin-3-yl)-1-oxoisoindolin-4-yl)hex-5-yn-1-yl)nicotinamido)-2-((R)-1-methylpyrrolidin-2-yl)-1H-pyrrolo[3,2-c]pyridine-1-carboxylate (Intermediate 72, 0.30 g, 0.34 mmol) in acetonitrile (8 mL) was added methanesulfonic acid (0.22 mL, 3.43 mmol) and the reaction stirred at 50° C. for 2 h. N,N′-dimethylethylenediamine (0.41 mL, 3.43 mmol) and triethylamine (0.96 mL, 6.86 mmol) were added at room temperature and the reaction mixture was stirred at room temperature for 2 h. The reaction mixture was poured into cold water (80 mL), resulting in a precipitate which was collected by filtration, washed with pentane (2×40 mL) and dried in vacuo to obtain the crude compound. The crude material was purified by preparative HPLC (Method A) to afford 6-(6-(2-(2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl) nicotinamide (Example 10, 0.09 g, 38%) as an off white solid. ¹H NMR (400 MHz, DMSO-d₆) δ ppm 1.60-1.68 (m, 2H), 1.76-1.95 (m, 5H), 1.97-2.06 (m, 1H), 2.12-2.14 (m, 1H), 2.17 (s, 3H), 2.24-2.30 (m, 1H), 2.41-2.46 (m, 1H), 2.56-2.62 (m, 3H), 2.84-2.92 (m, 3H), 3.10-3.19 (m, 1H), 3.34-3.41 (m, 1H), 4.28-4.36 (m, 1H), 4.43-4.50 (m, 1H), 5.11-5.16 (m, 1H), 6.40 (s, 1H), 7.38-7.42 (m, 1H), 7.50-7.55 (m, 1H), 7.64 (d, J=7.40 Hz, 1H), 7.71 (d, J=7.40 Hz, 1H), 8.19 (s, 1H), 8.27-8.30 (m, 1H), 8.51 (s, 1H), 9.08 (s, 1H), 10.68 (br s, 1H), 11.00 (br s, 1H), 11.40 (br s, 1H). Mass spec: m/z: 644.0 [M+H]⁺.

### Example 11 was Synthesised Following Scheme 14

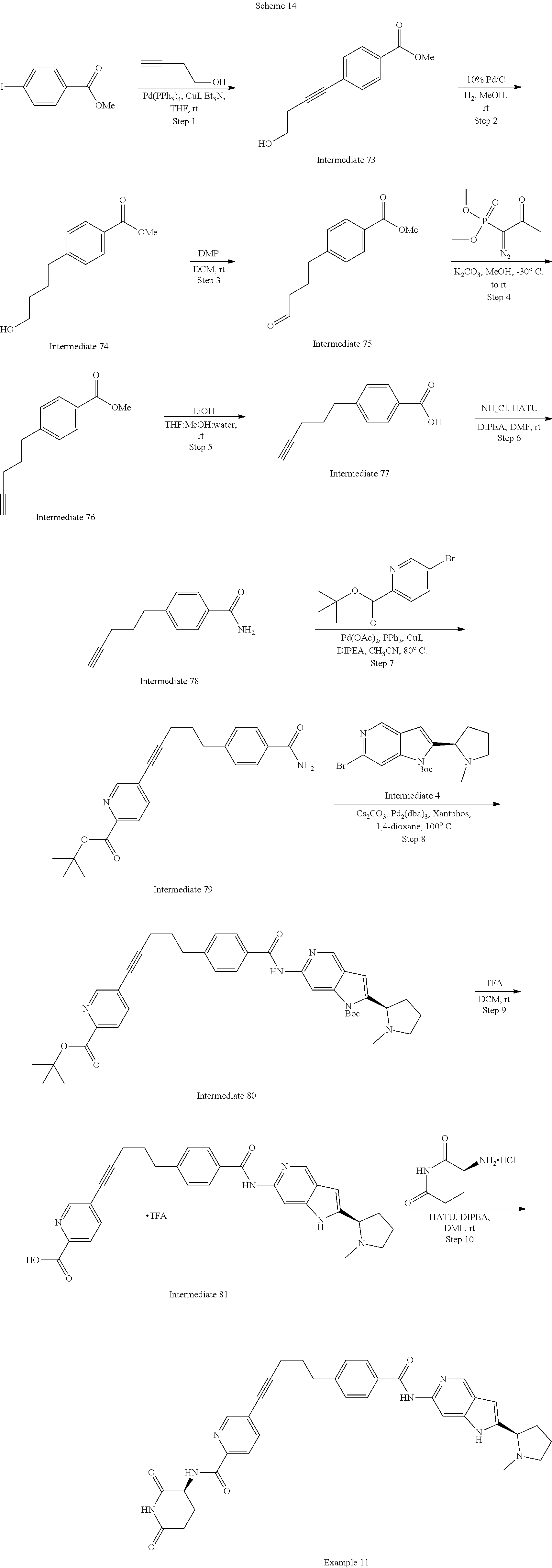

### Step 1

### Intermediate 73: methyl 4-(4-hydroxybut-1-yn-1-yl)benzoate

To a solution of methyl 4-iodobenzoate (CAS No: 619-44-3, 20.0 g, 76.32 mmol) in tetrahydrofuran (150 mL) was added but-3-yn-1-ol (CAS No: 927-74-2, 8.02 g, 114.49 mmol) followed by triethylamine (150 mL, 1070.0 mmol). The reaction mixture was purged with argon for 15 min followed by the addition of copper(I) iodide (1.48 g, 7.63 mmol) and Pd(PPh₃)₄ (4.41 g, 3.81 mmol). The reaction mixture was further purged with argon for 10 min and stirred at room temperature for 16 h. The reaction mixture was diluted with water (400 mL) and extracted with ethyl acetate (3×500 mL). The combined organic phases were dried over anhydrous Na₂SO₄ and concentrated in vacuo. The crude material was purified by combi-flash column chromatography by eluting with 80% ethyl acetate in heptane to afford methyl 4-(4-hydroxybut-1-yn-1-yl)benzoate (Intermediate 73, 14.00 g, 90%) as yellow solid. ¹H NMR (400 MHz, DMSO-d₆) δ ppm 2.59 (t, J=6.80 Hz, 2H), 3.56-3.62 (m, 2H), 3.85 (s, 3H), 4.93 (t, J=5.20 Hz, 1H), 7.52 (d, J=7.89 Hz, 2H), 7.91 (d, J=7.89 Hz, 2H). Mass spec: m/z: 205.4 [M+H]⁺.

### Step 2

### Intermediate 74: methyl 4-(4-hydroxybutyl)benzoate

To a solution of methyl 4-(4-hydroxybut-1-yn-1-yl)benzoate (Intermediate 73, 16.00 g, 78.35 mmol) in methanol (200 mL) was added 10% Pd/C (5.00 g) at room temperature. The reaction mixture was stirred at room temperature for 16 h under H₂ (110 psi). The reaction mixture was filtered through celite, washed with MeOH (300 mL) and concentrated in vacuo to afford methyl 4-(4-hydroxybutyl)benzoate (Intermediate 74, 15.0 g, 92%) as yellow liquid, which was used for the next step without further purification. ¹H NMR (400 MHz, DMSO-d₆) δ ppm 1.37-1.48 (m, 2H), 1.55-1.66 (m, 2H), 2.65 (t, J=7.60 Hz, 2H), 3.37-3.43 (m, 2H), 3.83 (s, 3H), 4.37 (t, J=5.20 Hz, 1H), 7.33 (d, J=8.0 Hz, 2H), 7.85-7.89 (d, J=8.0 Hz, 2H). Mass spec: m/z: 209.5 [M+H]⁺.

### Step 3

### Intermediate 75: methyl 4-(4-oxobutyl)benzoate

To a solution of methyl 4-(4-hydroxybutyl)benzoate (Intermediate 74, 14.00 g, 67.22 mmol) in dichloromethane (300 mL) was added Dess-Martin periodinane (44.09 g, 100.84 mmol) and the reaction mixture was stirred at room temperature for 4 h. The reaction mixture was diluted with mixture of saturated sodium bicarbonate and sodium thiosulphate (1:1, 500 mL) solution and extracted with dichloromethane (3×300 mL). The combined organic phases were dried over anhydrous Na₂SO₄ and concentrated in vacuo. The crude material was purified by combi-flash chromatography by eluting with 20% ethyl acetate in heptane to afford methyl 4-(4-oxobutyl)benzoate (Intermediate 75, 11.00 g, 82%) as a yellow liquid. ¹H NMR (400 MHz, DMSO-d₆) δ ppm 1.78-1.91 (m, 2H), 2.47 (t, J=7.20 Hz, 2H), 2.66 (t, J=7.20 Hz, 2H), 3.84 (s, 3H), 7.35 (d, J=8.0 Hz, 2H), 7.88 (d, J=8.0 Hz, 2H), 9.66 (s, 1H).

### Step 4

### Intermediate 76: methyl 4-(pent-4-yn-1-yl)benzoate

To a solution of methyl 4-(4-oxobutyl)benzoate (Intermediate 75, 11.00 g, 53.33 mmol) in methanol (200 mL) was added potassium carbonate (14.72 g, 106.67 mmol) at −30° C. Dimethyl (1-diazo-2-oxopropyl)phosphonate (Comp-6, 15.37 g, 80.0 mmol) was added and the reaction mixture stirred from −30° C. to room temperature and then at room temperature for 3 h. The reaction mixture was diluted water (500 mL) and extracted with ethyl acetate (3×200 mL). The combined organic phases were dried over anhydrous Na₂SO₄ and concentrated in vacuo. The crude material was purified by combi-flash chromatography by eluting with 10% ethyl acetate in heptane to afford methyl 4-(pent-4-yn-1-yl)benzoate (Intermediate 76, 8.00 g, 74%) as a yellow liquid. ¹H NMR (400 MHz, DMSO-d₆) δ ppm 1.73-1.91 (m, 2H), 2.14-2.18 (m, 2H), 2.73 (t, J=7.60 Hz, 2H), 2.82 (s, 1H), 3.83 (s, 3H), 7.35 (d, J=8.0 Hz, 2H), 7.88 (d, J=8.0 Hz, 2H).

### Step 5

### Intermediate 77: 4-(pent-4-yn-1-yl)benzoic acid

To a solution of methyl 4-(pent-4-yn-1-yl)benzoate (Intermediate 76, 8.00 g, 39.56 mmol) in tetrahydrofuran (40 mL), methanol (40 mL) and water (20 mL) was added lithium hydroxide (3.86 g, 158.3 mmol) in water (20 mL). The reaction mixture was then stirred at room temperature for 16 h. The reaction mixture was then concentrated in vacuo, the crude was then diluted with water (30 mL) and acidified to pH~4 with 0.5 N HCl. The resulting precipitate was filtered and dried in vacuo to afford 4-(pent-4-yn-1-yl)benzoic acid (Intermediate 77, 6.30 g, 85%) as a white solid, which was used for the next step without further purification. ¹H NMR (400 MHz, DMSO-d₆) δ ppm 1.72-1.80 (m, 2H), 2.12-2.20 (m, 2H), 2.73 (t, J=7.60 Hz, 2H), 2.82 (s, 1H), 7.32 (d, J=7.88 Hz, 2H), 7.86 (d, J=7.88 Hz, 2H), 12.80 (br s, 1H).

### Step 6

### Intermediate 78: 4-(pent-4-yn-1-yl)benzamide

To a solution of 4-(pent-4-yn-1-yl)benzoic acid (Intermediate 77, 6.30 g, 33.0 mmol) in dimethylformamide (80 mL) were added HATU (20 g, 50.0 mmol) and N,N-diisopropylethylamine (18 mL, 100.0 mmol). Ammonium chloride (7.2 g, 130.0 mmol) was then added and the reaction stirred at room temperature for 16 h. The reaction mixture was diluted water (100 mL) and extracted with ethyl acetate (3×100 mL). The combined organic phases were dried over anhydrous Na₂SO₄ and concentrated in vacuo. The crude material was purified by combi-flash chromatography by eluting with 80% ethyl acetate in heptane to afford 4-(pent-4-yn-1-yl)benzamide (Intermediate 78, 5.60 g, 89%) as an off white solid. ¹H NMR (400 MHz, DMSO-d₆) δ ppm 1.68-1.80 (m, 2H), 2.09-2.20 (m, 2H), 2.71 (t, J=7.60 Hz, 2H), 2.83 (s, 1H), 7.26 (br s, 1H), 7.27 (d, J=8.0 Hz, 2H), 7.79 (d, J=8.0 Hz, 2H), 7.89 (br s, 1H). Mass spec: 188.5 [M+H]⁺.

### Step 7

### Intermediate 79: tert-butyl 5-(5-(4-carbamoylphenyl)pent-1-yn-1-yl)picolinate

To a solution of 4-(pent-4-yn-1-yl)benzamide (Intermediate 78, 1.00 g, 5.34 mmol) in acetonitrile (20 mL) was added tert-butyl 5-bromopicolinate (CAS No: 845306-08-3, 1.38 g, 5.34 mmol) followed by N,N-diisopropylethylamine (5 mL, 26.704 mmol). The reaction mixture was purged with argon for 15 min followed by the addition of copper(I) iodide (0.11 g, 0.53 mmol), Pd(OAc)₂ (0.13 g, 0.80 mmol) and PPh₃ (0.42 g, 1.60 mmol). The reaction mixture was further purged with argon for 10 min and heated at 80° C. for 2 h. The reaction mixture was diluted with water (100 mL) and extracted with ethyl acetate (3×50 mL). The combined organic phases were dried over anhydrous Na₂SO₄ and concentrated in vacuo. The crude material was purified by combi-flash chromatography by eluting with 2% MeOH in DCM to afford tert-butyl 5-(5-(4-carbamoylphenyl)pent-1-yn-1-yl)picolinate (Intermediate 79, 1.20 g, 62%) as a yellow solid. ¹H NMR (400 MHz, DMSO-d₆) δ ppm 1.55 (s, 9H), 1.54-1.58 (m, 2H), 1.89 (t, J=7.60 Hz, 2H), 2.82 (t, J=7.60 Hz, 2H), 7.28 (br s, 1H), 7.31 (d, J=8.0 Hz, 2H), 7.80 (d, J=8.0 Hz, 2H), 7.87 (br s, 1H), 7.92-7.96 (m, 2H), 8.70 (s, 1H). Mass spec: m/z: 365.3 [M+H]⁺.

### Step 8

### Intermediate 80: tert-butyl (R)-6-(4-(5-(6-(tert-butoxycarbonyl) pyridin-3-yl)pent-4-yn-1-yl)benzamido)-2-(1-methylpyrrolidin-2-yl)-1H-pyrrolo[3,2-c]pyridine-1-carboxylate

To a solution of tert-butyl 5-(5-(4-carbamoylphenyl)pent-1-yn-1-yl)picolinate (Intermediate 79, 1.00 g, 2.7 mmol) in 1,4-dioxane (20 mL) was added tert-butyl (R)-6-bromo-2-(1-methylpyrrolidin-2-yl)-1H-pyrrolo[3,2-c]pyridine-1-carboxylate (Intermediate 4, 1.1 g, 3.0 mmol) followed by cesium carbonate (1.60 g, 8.2 mmol) at room temperature. The reaction mixture was purged with argon for 15 min followed by the addition of Pd₂(dba)₃ (0.39 g, 0.41 mmol) and Xantphos (0.48 g, 0.82 mmol). The reaction mixture was further purged with argon for 10 min and heated at 100° C. for 2 h. The reaction mixture was concentrated in vacuo and the crude material was purified by combi-flash chromatography by eluting with 70% ethyl acetate in heptane to afford tert-butyl (R)-6-(4-(5-(6-(tert-butoxycarbonyl)pyridin-3-yl)pent-4-yn-1-yl)benzamido)-2-(1-methylpyrrolidin-2-yl)-1H-pyrrolo[3,2-c]pyridine-1-carboxylate (Intermediate 80, 1.20 g, 66%) as a yellow solid. ¹H NMR (400 MHz, DMSO-d₆) δ ppm 1.55 (s, 9H), 1.58-1.64 (m, 2H), 1.70 (s, 9H), 1.74-1.82 (m, 2H), 1.86-2.00 (m, 3H), 2.11-2.20 (m, 2H), 2.35 (s, 3H), 2.78-2.88 (m, 2H), 3.12-3.18 (m, 1H), 3.88-3.96 (m, 1H), 6.75 (s, 1H), 7.26-7.46 (m, 3H), 7.96-8.02 (m, 3H), 8.59 (s, 1H), 8.71 (s, 1H), 8.93 (s, 1H), 10.60 (br s, 1H). Mass spec: m/z: 664.1 [M+H]⁺.

### Step 9

### Intermediate 81: (R)-5-(5-(4-((2-(1-methylpyrrolidin-2-yl)-1H-pyrrolo[3,2-c]pyridin-6-yl)carbamoyl)phenyl) pent-1-yn-1-yl)picolinic acid trifluoroacetate

To a solution of tert-butyl (R)-6-(4-(5-(6-(tert-butoxycarbonyl)pyridin-3-yl)pent-4-yn-1-yl)benzamido)-2-(1-methylpyrrolidin-2-yl)-1H-pyrrolo[3,2-c]pyridine-1-carboxylate (Intermediate 80, 0.50 g, 0.75 mmol) in dichloromethane (10 mL) was added trifluoroacetic acid (10 mL) at 0° C. The reaction mixture was then stirred at room temperature for 16 h. The reaction mixture was concentrated in vacuo, the crude material was washed with diethyl ether (5×2 mL) and dried under reduced pressure to afford (R)-5-(5-(4-((2-(1-methylpyrrolidin-2-yl)-1H-pyrrolo[3,2-c]pyridin-6-yl)carbamoyl)phenyl)pent-1-yn-1-yl)picolinic acid trifluoroacetate (Intermediate 81, 0.50 g) as a gum, which was used for the next step without further purification. Mass spec: m/z: 508.2 [M+H]⁺.

### Step 10

### Example 11: N—((S)-2,6-dioxopiperidin-3-yl)-5-(5-(4-((2-((R)-1-methylpyrrolidin-2-yl)-1H-pyrrolo[3,2-c]pyridin-6-yl)carbamoyl)phenyl)pent-1-yn-1-yl)picolinamide

To a solution of (R)-5-(5-(4-((2-(1-methylpyrrolidin-2-yl)-1H-pyrrolo[3,2-c]pyridin-6-yl)carbamoyl)phenyl)pent-1-yn-1-yl)picolinic acid trifluoroacetate (Intermediate 81, 0.40 g, 0.78 mmol) in dimethylformamide (10 mL) was added HATU (0.47 g, 1.18 mmol) and the reaction stirred at room temperature for 15 min. (S)-3-aminopiperidine-2,6-dione hydrochloride (CAS No: 25181-50-4, 0.25 g, 1.18 mmol) and N,N-diisopropylethylamine (0.55 mL, 3.15 mmol) were added and the reaction mixture was stirred at room temperature for 3 h. The reaction mixture was quenched with ice cold water (100 mL) to afford a precipitate which was filtered and dried in vacuo to obtain crude compound. The crude material was purified by preparative HPLC (Method A) to afford N—((S)-2,6-dioxopiperidin-3-yl)-5-(5-(4-((2-((R)-1-methylpyrrolidin-2-yl)-1H-pyrrolo[3,2-c]pyridin-6-yl)carbamoyl)phenyl)pent-1-yn-1-yl)picolinamide (Example 11, 0.09 g, 20%) as a white solid. ¹H NMR (400 MHz, DMSO-d₆) δ ppm 1.78-2.03 (m, 6H), 2.17 (s, 3H), 2.19-2.30 (m, 3H), 2.44-2.48 (m, 2H), 2.54-2.57 (m, 2H), 2.75-2.87 (m, 3H), 3.12-3.16 (m, 1H), 4.74-4.83 (m, 1H), 6.39 (s, 1H), 7.39 (d, J=8.0 Hz, 2H), 7.98-8.07 (m, 4H), 8.19 (s, 1H), 8.50 (s, 1H), 8.70 (s, 1H), 9.08 (d, J=8.0 Hz, 1H), 10.36 (br s, 1H), 10.86 (br s, 1H), 11.35 (br s, 1H). Mass spec: m/z: 618.2 [M+H]⁺.

### Example 12 was Synthesised Following Scheme 15

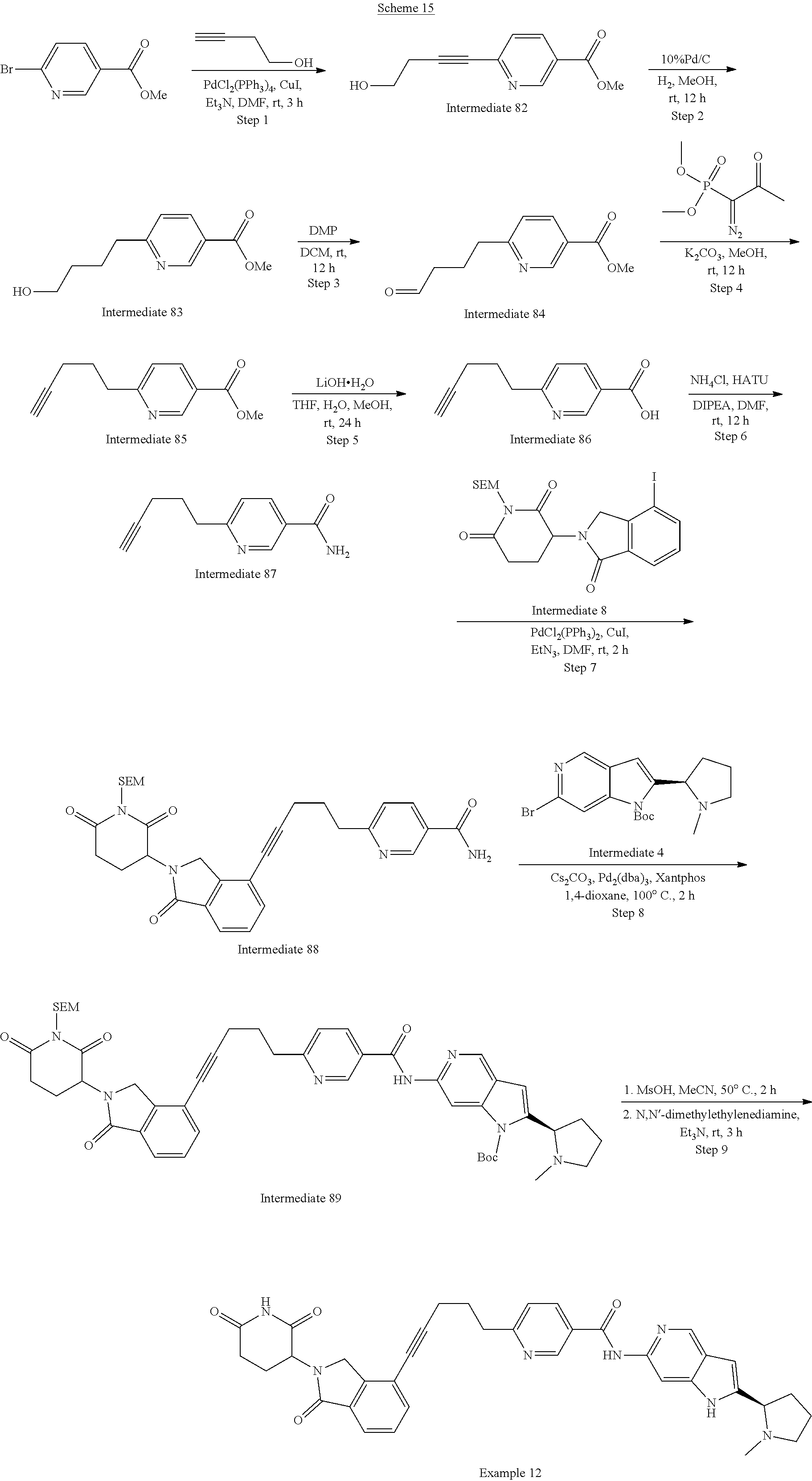

### Step 1

### Intermediate 82: methyl 6-(4-hydroxybut-1-yn-1-yl)nicotinate

To a solution of methyl 6-bromonicotinate (CAS No: 26218-78-0, 25.0 g, 115.7 mmol) in dimethylformamide (150 mL) was added but-3-yn-1-ol (CAS No: 927-74-2, 8.11 g, 115.7 mmol) followed by triethylamine (48.6 mL, 347.2 mmol). The reaction mixture was purged with argon for 15 min. Copper(I) iodide (2.23 g, 11.57 mmol) and PdCl₂(PPh₃)₂ (8.37 g, 11.57 mmol) were then added and the reaction mixture was further purged with argon for 10 min and stirred at room temperature for 3 h. The reaction mixture was diluted with water (500 mL) and extracted with ethyl acetate (3×500 mL). The combined organic phases were dried over anhydrous Na₂SO₄ and concentrated in vacuo. The crude material was purified by combi-flash column chromatography by eluting with 100% ethyl acetate in heptane to afford methyl 6-(4-hydroxybut-1-yn-1-yl) nicotinate (Intermediate 82, 14.0 g, 59%) as an off white solid. ¹H NMR (400 MHz, DMSO-d₆) δ ppm 2.63 (t, J=6.80 Hz, 2H), 3.59-3.64 (m, 2H), 3.88 (s, 3H), 4.98 (t, J=6.80 Hz, 1H), 7.60 (d, J=8.40 Hz, 1H), 8.24 (d, J=8.40 Hz, 1H), 9.01 (s, 1H). Mass spec: m/z: 206.01 [M+H]⁺.

### Step 2

### Intermediate 83: methyl 6-(4-hydroxybutyl)nicotinate

To a solution of methyl 6-(4-hydroxybut-1-yn-1-yl)nicotinate (Intermediate 82, 13.0 g, 63.35 mmol) in methanol (150 mL) was added 10% Pd/C (4.5 g) and the mixture was stirred at room temperature for 12 h under hydrogen (100 psi). After completion, the reaction mixture was filtered through a celite bed and concentrated in vacuo to afford methyl 6-(4-hydroxybutyl)nicotinate (Intermediate 83, 9.00 g, 70%) as yellow liquid, which was used for the next step without further purification. ¹H NMR (400 MHz, DMSO-d₆) δ ppm 1.34-1.50 (m, 2H), 1.66-1.76 (m, 2H), 2.81 (t, J=7.60 Hz, 2H), 3.38-3.43 (m, 2H), 3.87 (s, 3H), 4.38 (t, J=5.20 Hz, 1H), 7.42 (d, J=8.40 Hz, 1H), 8.18 (d, J=8.40 Hz, 1H), 8.99 (s, 1H). Mass spec: m/z: 208.02 [M−H]⁻.

### Step 3

### Intermediate 84: methyl 6-(4-oxobutyl)nicotinate

To a solution of methyl 6-(4-hydroxybutyl)nicotinate (Intermediate 83, 9.00 g, 43.01 mmol) in dichloromethane (90 mL) was added Dess-Martin Periodinane (22.11 g, 51.61 mmol) and the mixture was stirred at room temperature for 12 h. The reaction mixture was then filtered through celite and concentrated in vacuo to afford methyl 6-(4-oxobutyl) nicotinate (Intermediate 84, 2.70 g, 30%) as a yellow liquid, which was used for the next step without further purification. ¹H NMR (400 MHz, DMSO-d₆) δ ppm 1.84-2.00 (m, 2H), 2.82 (t, J=7.60, 2H), 3.32-3.35 (m, 2H), 3.87 (s, 3H), 7.37-7.48 (m, 1H), 8.08-8.24 (m, 1H), 9.00 (s, 1H), 9.69 (s, 1H).

### Step 4

### Intermediate 85: methyl 6-(pent-4-yn-1-yl)nicotinate

To a solution of methyl 6-(4-oxobutyl) nicotinate (Intermediate 84, 2.70 g, 13.0 mmol) in methanol (20 mL) was added potassium carbonate (1.90 g, 20.0 mmol). Dimethyl(1-diazo-2-oxopropyl)phosphonate (3.0 g, 16.0 mmol) was added and the reaction stirred at room temperature for 12 h. The reaction mixture was concentrated in vacuo and the crude material was purified by combi-flash column chromatography by eluting with 25% ethyl acetate in heptane to afford methyl 6-(pent-4-yn-1-yl) nicotinate (Intermediate 85, 1.70 g, 64%) as colourless liquid. ¹H NMR (400 MHz, DMSO-d₆) δ ppm 1.84-1.92 (m, 2H), 2.20 (t, J=7.60 Hz, 2H), 2.81 (s, 1H), 2.90 (t, J=7.60 Hz 2H), 3.87 (s, 3H), 7.43 (d, J=8.40 Hz, 1H), 8.19 (d, J=8.40 Hz, 1H), 9.00 (s, 1H). Mass spec: m/z 204.04 [M+H]⁺.

### Step 5

### Intermediate 86: 6-(pent-4-yn-1-yl)nicotinic acid

To a solution of methyl 6-(pent-4-yn-1-yl)nicotinate (Intermediate 85, 1.70 g, 8.4 mmol) in tetrahydrofuran: methanol:water (3:1:1, 50 mL) was added lithium hydroxide (0.61 g, 25.0 mmol) in water (10 mL) at 0° C. The reaction mixture was then stirred at room temperature for 24 h. The reaction mixture was concentrated in vacuo. The reaction mixture was diluted with water (50 mL), acidified to pH~4 with 1N HCl and extracted with ethyl acetate (3×100 mL). The combined organic phases were dried over anhydrous Na₂SO₄, and concentrated in vacuo to afford 6-(pent-4-yn-1-yl)nicotinic acid (Intermediate 86, 1.50 g, 95%) as an off white solid. ¹H NMR (401 MHz, DMSO-d₆) δ ppm 1.84-1.92 (m, 2H), 2.18-2.24 (m, 2H), 2.84 (s, 1H), 2.90 (t, J=7.60 Hz, 2H), 7.41 (d, J=8.0 Hz, 1H), 8.17 (d, J=8.0 Hz, 1H), 8.98 (s, 1H), 13.28 (br s, 1H). Mass spec: m/z: 189.98 [M+H]⁺.

### Step 6

### Intermediate 87: 6-(pent-4-yn-1-yl)nicotinamide

To a solution of 6-(pent-4-yn-1-yl)nicotinic acid (Intermediate 86, 1.50 g, 7.93 mmol) in dimethylformamide (10 mL) was added HATU (3.73 g, 9.51 mmol) and N,N-diisopropylethylamine (5.54 mL, 31.7 mmol). Ammonium chloride (1.70 g, 31.7 mmol) was then added and the reaction stirred at room temperature for 12 h. The reaction mixture was diluted with water (100 mL) and extracted with ethyl acetate (3×200 mL). The combined organic phases were dried over anhydrous Na₂SO₄, and concentrated in vacuo. The crude material was purified by combi-flash chromatography by eluting with 100% ethyl acetate in heptane to afford 6-(pent-4-yn-1-yl)nicotinamide (Intermediate 87, 1.40 g, 94%) as an off white solid. Mass spec: m/z: 189.06 [M+H]⁺.

### Step 7

### Intermediate 88: 6-(5-(2-(2,6-dioxo-1-((2-(trimethylsilyl)ethoxy)methyl)piperidin-3-yl)-1-oxoisoindolin-4-yl) pent-4-yn-1-yl)nicotinamide

To a solution of 6-(pent-4-yn-1-yl) nicotinamide (Intermediate 87, 0.60 g, 3.18 mmol) in dimethylformamide (5 mL) was added 3-(4-iodo-1-oxoisoindolin-2-yl)-1-((2-(trimethylsilyl)ethoxy)methyl)piperidine-2,6-dione (Intermediate 8, 1.59 g, 3.18 mmol) followed by triethylamine (16.1 mL, 114.75 mmol). The reaction mixture was purged with argon for 15 min followed by the addition of copper(I) iodide (0.06 g, 0.31 mmol) and PdCl₂(PPh₃)₂ (0.23 g, 0.31 mmol). The reaction mixture was further purged with argon for 10 min and stirred at room temperature for 2 h. The reaction mixture was diluted with water (100 mL) and extracted with ethyl acetate (3×100 mL). The combined organic phases were dried over anhydrous Na₂SO₄, and concentrated in vacuo. The crude material was purified by combi-flash chromatography by eluting with ethyl acetate to afford 6-(5-(2-(2,6-dioxo-1-((2-(trimethylsilyl)ethoxy)methyl)piperidin-3-yl)-1-oxoisoindolin-4-yl)pent-4-yn-1-yl)nicotinamide (Intermediate 88, 0.25 g, 10%) as a yellow solid. ¹H NMR (400 MHz, DMSO-d₆) δ ppm −0.05 (s, 9H), 0.75-0.89 (m, 2H), 1.94-2.11 (m, 3H), 2.36-2.44 (m, 2H), 2.52-2.56 (m, 2H), 2.90-2.96 (m, 2H), 3.02-3.12 (m, 1H), 3.44-3.56 (m, 2H), 4.26-4.30 (m, 1H), 4.47-4.52 (m, 1H), 4.99-5.10 (m, 2H), 5.25-5.30 (m, 1H), 7.38 (d, J=7.60 Hz, 1H), 7.51 (br s, 1H), 7.53 (d, J=7.60 Hz, 1H), 7.65 (d, J=7.60 Hz, 1H), 7.72 (d, J=7.60 Hz, 1H), 8.07 (br s, 1H), 8.11-8.14 (m, 1H), 8.94 (s, 1H). Mass spec: m/z: 561.3 [M+H]⁺.

### Step 8

### Intermediate 89: tert-butyl 6-(6-(5-(2-(2,6-dioxo-1-((2-(trimethylsilyl)ethoxy)methyl)piperidin-3-yl)-1-oxoisoindolin-4-yl)pent-4-yn-1-yl)nicotinamido)-2-((R)-1-methylpyrrolidin-2-yl)-1H-pyrrolo[3,2-c]pyridine-1-carboxylate

To a solution of 6-(5-(2-(2,6-dioxo-1-((2-(trimethylsilyl)ethoxy)methyl)piperidin-3-yl)-1-oxoisoindolin-4-yl) pent-4-yn-1-yl)nicotinamide (Intermediate 88, 0.50 g, 0.9 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.30 g, 0.9 mmol) followed by cesium carbonate (0.6 g, 2.0 mmol) at room temperature. The reaction mixture was purged with argon for 15 min followed by Pd₂(dba)₃ (0.1 g, 0.1 mmol) and Xantphos (0.2 g, 0.3 mmol). The reaction mixture was purged with argon for 10 min and heated at 100° C. for 2 h. Progress of the reaction was monitored by TLC. After completion, the reaction mixture was concentrated in vacuo. The crude material was purified by combi-flash chromatography by eluting with 90% ethyl acetate in heptane to afford tert-butyl 6-(6-(5-(2-(2,6-dioxo-1-((2-(trimethylsilyl)ethoxy)methyl)piperidin-3-yl)-1-oxoisoindolin-4-yl)pent-4-yn-1-yl)nicotinamido)-2-((R)-1-methylpyrrolidin-2-yl)-1H-pyrrolo[3,2-c]pyridine-1-carboxylate (Intermediate 89, 0.40 g, 50%) as white solid. ¹H NMR (400 MHz, DMSO-d₆) δ ppm −0.06 (s, 9H), 0.75-0.87 (m, 2H), 1.69 (s, 9H), 1.72-1.79 (m, 3H), 2.01-2.08 (m, 3H), 2.35 (s, 3H), 2.38-2.43 (m, 1H), 2.52-2.58 (m, 4H), 2.92-3.08 (m, 3H), 3.09-3.17 (m, 2H), 3.47-3.55 (m, 2H), 3.89-3.95 (m, 1H), 4.27-4.32 (m, 1H), 4.48-4.54 (m, 1H), 4.99-5.10 (m, 2H), 5.25-5.30 (m, 1H), 6.76 (s, 1H), 7.45 (d, J=8.40 Hz, 1H), 7.51-7.56 (m, 1H), 7.66-7.68 (m, 1H), 7.72-7.74 (m, 1H), 8.28-8.30 (m, 1H), 8.60 (s, 1H), 8.92 (s, 1H), 9.08 (s, 1H), 10.92 (br s, 1H). Mass spec: m/z: 860.2 [M+H]⁺.

### Step 9

### Example 12: 6-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)nicotinamide

To a solution of tert-butyl 6-(6-(5-(2-(2,6-dioxo-1-((2-(trimethylsilyl)ethoxy)methyl)piperidin-3-yl)-1-oxoisoindolin-4-yl)pent-4-yn-1-yl)nicotinamido)-2-((R)-1-methylpyrrolidin-2-yl)-1H-pyrrolo[3,2-c]pyridine-1-carboxylate (Intermediate 89, 0.40 g, 0.5 mmol) in acetonitrile (4 mL) was added methane sulfonic acid (0.5 mL, 7.0 mmol) at room temperature and heated at 50° C. for 2 h. N¹,N²-dimethylethane-1,2-diamine (0.3 mL, 3.0 mmol) followed by triethylamine (0.9 g, 9.0 mmol) were added and the reaction was stirred at room temperature for 3 h. The reaction mixture was diluted with water (100 mL), resulting in a precipitate which was filtered and dried in vacuo to obtain the crude compound. The crude material was purified by preparative HPLC (Method A) to afford 6-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)nicotinamide (Example 12, 0.11 g, 40%) as an off white solid. ¹H NMR (400 MHz, DMSO-d₆) δ ppm 1.76-1.95 (m, 3H), 1.97-2.09 (m, 3H), 2.17 (s, 3H), 2.22-2.31 (m, 2H), 2.42-2.46 (m, 1H), 2.53-2.63 (m, 4H), 2.85-3.03 (m, 3H), 3.09-3.19 (m, 1H), 4.31-4.39 (m, 1H), 4.45-4.53 (m, 1H), 5.12-5.16 (m, 1H), 6.40 (s, 1H), 7.44 (d, J=8.0 Hz, 1H), 7.50-7.56 (m, 1H), 7.64-7.67 (m, 1H), 7.68-7.74 (m, 1H), 8.19 (s, 1H), 8.29-8.32 (m, 1H), 8.51 (s, 1H), 9.10 (s, 1H), 10.68 (br s, 1H), 11.00 (br s, 1H), 11.39 (br s, 1H). Mass spec: m/z: 630.0 [M+H]⁺.

### Example 13 was Synthesized Following Scheme 16

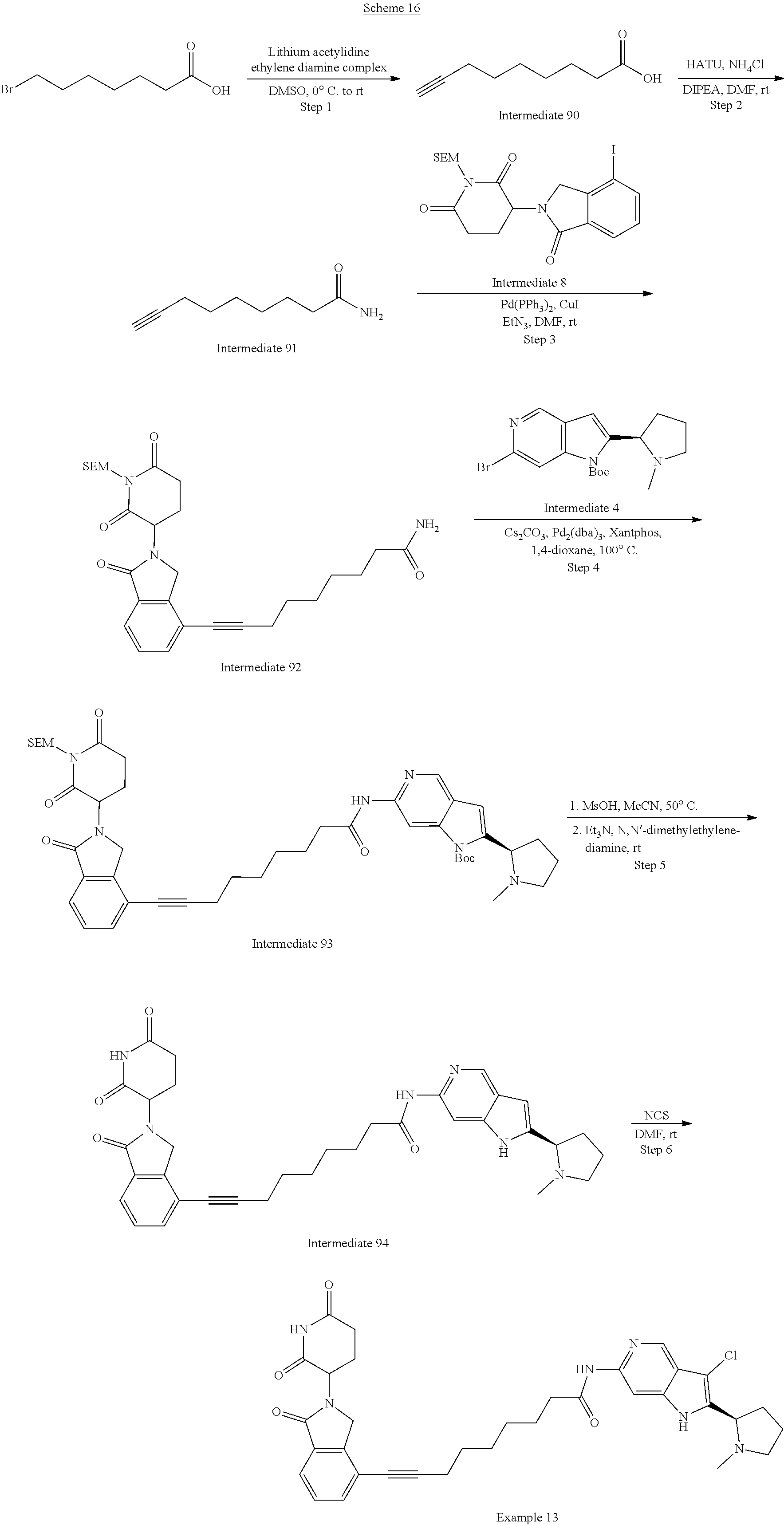

### Step 1

### Intermediate 90: non-8-ynoic acid

To solution of 7-bromoheptanoic acid (CAS No: 30515-28-7, 6.00 g, 28.69 mmol) in anhydrous DMSO (9 mL) was added dropwise over 30 min to a suspension of lithium acetylide ethylenediamine complex (11.74 g, 114.79 mmol) in anhydrous DMSO (90 mL) at 0° C. and reaction mixture was stirred at 0° C. for 1 h. The reaction mixture was stirred then stirred at room temperature for 2 h. The reaction mixture was quenched with 10% sulfuric acid solution (250 mL) at 0° C. The aqueous layer was extracted with hexane (4×100 mL) and the combined organic layer was dried over anhydrous Na₂SO₄, filtered, and concentrated in vacuo to afford non-8-ynoic acid (Intermediate 90, 3.7 g, 84%) as a pink oil, which was used for the next step without further purification. ¹H NMR (400 MHz, CDCl₃) δ ppm 1.336-1.48 (m, 4H), 1.52-1.57 (m, 2H), 1.62-1.69 (m, 2H), 1.95 (s, 1H), 2.16-2.21 (m, 2H), 2.36 (t, J=7.45 Hz, 2H).

### Step 2

### Intermediate 91: non-8-ynamide

To solution of non-8-ynoic acid (Intermediate 90, 3.9 g, 25 mmol) in dimethylformamide (20 mL) was added HATU (15 g, 38 mmol) and N,N-diisopropylethylamine (18 mL, 100 mmol) and stirred for 10 min at room temperature. Ammonium chloride (4.4 mL, 130 mmol) was then added and the reaction mixture was stirred at room temperature for 16 h. The reaction mixture was diluted with ice cold water (100 mL) and extracted with ethyl acetate (2×50 mL). The combined organic phases were dried over anhydrous Na₂SO₄, filtered, and concentrated in vacuo to obtain crude compound. The crude material was poured in water (50 mL) and stirred for 10 min, upon which time a solid precipitated, which was filtered and dried, to afford non-8-ynamide (Intermediate 91, 2.5 g, 65%) as an off white solid. ¹H NMR (400 MHz, DMSO-d₆) δ ppm 1.20-1.28 (m, 2H), 1.29-1.37 (m, 2H), 1.39-1.50 (m, 4H), 2.02 (t, J=7.45 Hz, 2H), 2.12-2.16 (m, 2H), 2.72 (s, 1H), 6.65 (br s, 1H), 7.20 (br s, 1H).

### Step 3

### Intermediate 92: 9-(2-(2,6-dioxo-1-((2-(trimethylsilyl)ethoxy)methyl) piperidin-3-yl)-1-oxoisoindolin-4-yl) non-8-ynamide

To a solution of non-8-ynamide (Intermediate 91, 2.40 g, 15.7 mmol) in dimethylformamide (20 mL) was added 3-(4-iodo-1-oxoisoindolin-2-yl)-1-((2-(trimethylsilyl)ethoxy)methyl)piperidine-2,6-dione (Intermediate 8, 7.84 g, 15.7 mmol) followed by triethylamine (79 mL, 564 mmol) and the reaction mixture was purged with argon gas for 15 min. PdCl₂(PPh₃)₂ (1.10 g, 1.57 mmol) and Copper(I) iodide (0.63 g, 3.13 mmol) were then added at room temperature and the reaction mixture was further purged with argon for 10 min and stirred at room temperature for 2 h. The reaction mixture was quenched with ice cold water (100 mL) and extracted with ethyl acetate (2×100 mL). The combined organic phases were dried over anhydrous Na₂SO₄, filtered, and concentrated in vacuo. The crude material was purified by combi-flash column chromatography, by eluting with 11% of MeOH in DCM, to afford 9-(2-(2,6-dioxo-1-((2-(trimethylsilyl)ethoxy)methyl)piperidin-3-yl)-1-oxoisoindolin-4-yl)non-8-ynamide (Intermediate 92, 2.7 g, 33%) as light brown solid. ¹H NMR (400 MHz, DMSO-d₆) δ ppm −0.02 (s, 9H), 0.78-0.90 (m, 2H), 1.27-1.32 (m, 2H), 1.40-1.60 (m, 6H), 1.99-2.10 (m, 3H), 2.41-2.46 (m, 3H), 2.78-2.82 (m, 1H), 3.02-3.11 (m, 1H), 3.51-3.56 (m, 2H), 4.25-4.29 (m, 1H), 4.44-4.49 (m, 1H), 5.01-5.09 (m, 2H), 5.24-5.29 (m, 1H), 6.64 (br s, 1H), 7.19 (br s, 1H), 7.48-7.57 (m, 1H), 7.64 (d, J=7.46 Hz, 1H), 7.72 (d, J=7.46 Hz, 1H). Mass spec: m/z: 523.9 [M−H]⁻.

### Step 4

### Intermediate 93: tert-butyl 6-(9-(2-(2,6-dioxo-1-((2-(trimethylsilyl)ethoxy)methyl)piperidin-3-yl)-1-oxoisoindolin-4-yl)non-8-ynamido)-2-((R)-1-methylpyrrolidin-2-yl)-1H-pyrrolo[3,2-c]pyridine-1-carboxylate

To a solution of 9-(2-(2,6-dioxo-1-((2-(trimethylsilyl) ethoxy)methyl) piperidin-3-yl)-1-oxoisoindolin-4-yl) non-8-ynamide (Intermediate 92, 2.45 g, 4.66 mmol) and tert-butyl (R)-6-bromo-2-(1-methylpyrrolidin-2-yl)-1H-pyrrolo[3,2-c]pyridine-1-carboxylate (Intermediate 4, 2.13 g, 5.59 mmol) in 1,4-dioxane (40 mL) was added cesium carbonate (4.60 g, 14.0 mmol) and the reaction mixture was purged with argon for 15 min. Pd₂(dba)₃ (0.66 g, 0.69 mmol) and xantphos (0.83 g, 1.40 mmol) were added at room temperature and the reaction mixture was purged with argon gas for a further 10 min and then heated at 100° C. for 2 h. The reaction mixture was concentrated in vacuo to obtain crude compound. The crude material was purified by combi-flash column chromatography, by eluting with 7% MeOH in DCM, to afford tert-butyl 6-(9-(2-(2,6-dioxo-1-((2-(trimethylsilyl)ethoxy)methyl)piperidin-3-yl)-1-oxoisoindolin-4-yl)non-8-ynamido)-2-((R)-1-methylpyrrolidin-2-yl)-1H-pyrrolo[3,2-c]pyridine-1-carboxylate (Intermediate 93, 1.95 g, 51%) as a light brown solid. Mass spec: m/z: 825.1 [M+H]⁺.

### Step 5

### Intermediate 94: 9-(2-(2,6-dioxopiperidin-3-yl)-1-oxoisoindolin-4-yl)-N-(2-((R)-1-methylpyrrolidin-2-yl)-1H-pyrrolo[3,2-c]pyridin-6-yl) non-8-ynamide

To a solution of tert-butyl 6-(9-(2-(2,6-dioxo-1-((2-(trimethylsilyl)ethoxy)methyl)piperidin-3-yl)-1-oxoisoindolin-4-yl)non-8-ynamido)-2-((R)-1-methylpyrrolidin-2-yl)-1H-pyrrolo[3,2-c]pyridine-1-carboxylate (Intermediate 93, 1.9 g, 2.3 mmol) in acetonitrile (25 mL) was added methanesulfonic acid (1.5 mL, 23 mmol) at room temperature and the reaction mixture was heated at 50° C. for 2 h. N,N′-dimethylethylenediamine (1.4 mL, 12 mmol) followed by triethylamine (6.5 mL, 46 mmol) were added and the reaction stirred at room temperature for 3 h. The reaction mixture was quenched with water (100 mL), a solid precipitated which was filtered and washed with water (20 mL) and then dried to afford 9-(2-(2,6-dioxopiperidin-3-yl)-1-oxoisoindolin-4-yl)-N-(2-((R)-1-methylpyrrolidin-2-yl)-1H-pyrrolo[3,2-c]pyridin-6-yl)non-8-ynamide (Intermediate 94, 1.30 g, 95%) as an off white solid. ¹H NMR (400 MHz, DMSO-d₆) δ ppm 1.32-1.38 (m, 2H), 1.42-1.48 (m, 2H), 1.55-1.70 (m, 5H), 1.76-1.90 (m, 3H), 1.99-2.05 (m, 1H), 2.08-2.11 (m, 1H), 2.14 (s, 3H), 2.21-2.28 (m, 1H), 2.35-2.39 (m, 3H), 2.54-2.67 (m, 2H), 2.84-2.96 (m, 2H), 3.12 (t, J=7.46 Hz, 1H), 4.29-4.36 (m, 1H), 4.43-4.48 (m, 1H), 5.11-5.16 (m, 1H), 6.33 (s, 1H), 7.48-7.52 (m, 1H), 7.62 (d, J=7.46 Hz, 1H), 7.69 (d, J=7.46 Hz, 1H), 8.07 (s, 1H), 8.40 (s, 1H), 10.08 (br s, 1H), 10.99 (br s, 1H), 11.23 (br s, 1H). Mass spec: 595.38 [M+H]⁺.

### Step 6

### Example 13: N-(3-chloro-2-((R)-1-methylpyrrolidin-2-yl)-1H-pyrrolo[3,2-c]pyridin-6-yl)-9-(2-(2,6-dioxopiperidin-3-yl)-1-oxoisoindolin-4-yl) non-8-ynamide

To a solution of 9-(2-(2,6-dioxopiperidin-3-yl)-1-oxoisoindolin-4-yl)-N-(2-((R)-1-methylpyrrolidin-2-yl)-1H-pyrrolo[3,2-c]pyridin-6-yl)non-8-ynamide (Intermediate 94, 0.50 g, 0.84 mmol) in dimethylformamide (20 mL) was added N-chlorosuccinimide (0.17 g, 1.26 mmol) and the reaction mixture was stirred at room temperature for 16 h. The reaction mixture was quenched with ice cold water (100 mL) and extracted with ethyl acetate (2×100 mL). The combined organic phases were dried over anhydrous Na₂SO₄, filtered, and concentrated in vacuo. The crude material was purified by preparative HPLC (Method A) to afford N-(3-chloro-2-((R)-1-methylpyrrolidin-2-yl)-1H-pyrrolo[3,2-c]pyridin-6-yl)-9-(2-(2,6-dioxopiperidin-3-yl)-1-oxoisoindolin-4-yl)non-8-ynamide (Example 13, 0.06 g, 11%) as an off white solid. ¹H NMR (400 MHz, DMSO-d₆) δ ppm 1.33-1.40 (m, 2H), 1.43-1.52 (m, 2H), 1.55-1.65 (m, 4H), 1.80-2.02 (m, 4H), 2.14 (s, 3H), 2.27-2.34 (m, 1H), 2.36-2.41 (m, 3H), 2.57-2.60 (m, 1H), 2.85-2.93 (m, 1H), 3.13-3.18 (m, 2H), 3.45-3.52 (m, 2H), 4.29-4.34 (m, 2H), 4.43-4.48 (m, 1H), 5.11-5.16 (m, 1H), 7.50 (t, J=7.03 Hz, 1H), 7.63 (d, J=7.25 Hz, 1H), 7.70 (d, J=7.50 Hz, 1H), 8.13 (s, 1H), 8.40 (s, 1H), 10.26 (s, 1H), 10.98 (br s, 1H), 11.56 (br s, 1H). Mass spec: m/z: 629.2 [M+H]⁺.

### Example 14 was Synthesised Following Scheme 17

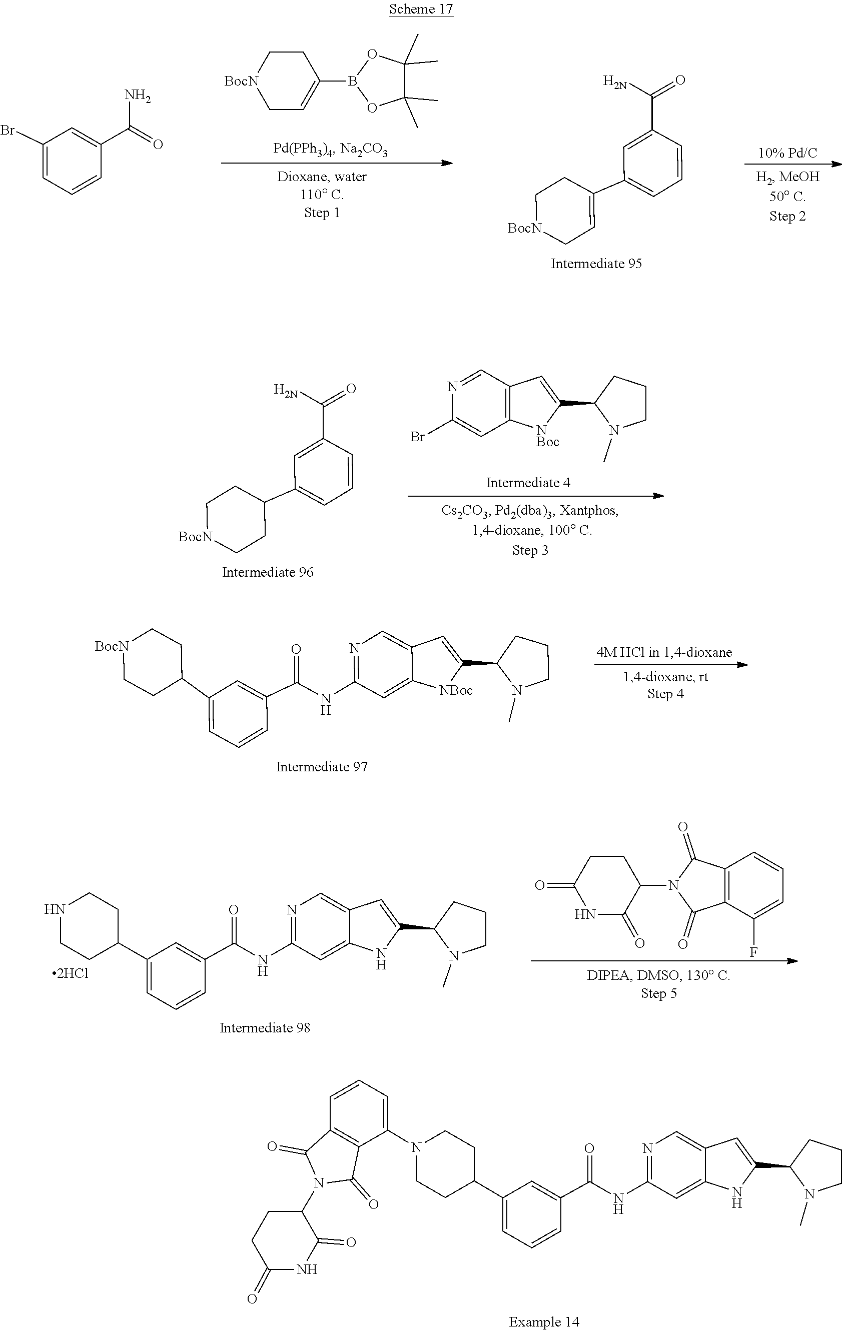

### Step 1

### Intermediate 95: tert-butyl 4-(3-carbamoylphenyl)-3,6-dihydropyridine-1(2H)-carboxylate

To a solution of tert-butyl 4-(4,4,5,5-tetramethyl-1,3,2-dioxaborolan-2-yl)-3,6-dihydropyridine-1(2H)-carboxylate (CAS No: 286961-14-6, 2.80 g, 9.0 mmol) in 1,4-dioxane (16 mL) and water (4 mL) was added 3-bromobenzamide (CAS No: 22726-00-7, 1.50 g, 7.5 mmol) followed by Na₂CO₃ (1.7 g, 16 mmol) and the reaction mixture was purged with argon for 15 min. Pd(PPh₃)₄ (0.45 g, 0.37 mmol) was added and the reaction mixture was further purged with argon for 10 min and then heated at 110° C. for 16 h. The reaction mixture was concentrated in vacuo to obtain crude residue, which was purified by combi-flash chromatography, by eluting with 30% ethyl acetate in heptane, to afford tert-butyl 4-(3-carbamoylphenyl)-3,6-dihydropyridine-1(2H)-carboxylate (Intermediate 95, 1.5 g, 66%) as an off white solid. ¹H NMR (400 MHz, DMSO-d₆) δ ppm 1.43 (s, 9H), 3.29-3.36 (m, 2H), 3.52-3.56 (m, 2H), 3.95-4.10 (m, 2H), 6.21-6.24 (m, 1H), 7.37 (br s, 1H), 7.40-7.44 (m, 1H) 7.56-7.64 (m, 1H), 7.75-7.79 (m, 1H), 7.92 (s, 1H), 8.01 (br s, 1H). Mass spec: m/z: 203.03 [M+H]⁺.

### Step 2

### Intermediate 96: tert-butyl 4-(3-carbamoylphenyl) piperidine-1-carboxylate

To a solution of tert-butyl 4-(3-carbamoylphenyl)-3,6-dihydropyridine-1(2H)-carboxylate (Intermediate 95, 1.5 g, 5.0 mmol) in methanol (30 mL) was added 10% Pd/C (0.4 g) and the reaction mixture was stirred at room temperature for 16 h under 100 psi H₂ atmosphere. The reaction mixture was filtered through celite and washed with MeOH (3×30 mL). The filtrate was concentrated in vacuo to afford tert-butyl 4-(3-carbamoylphenyl) piperidine-1-carboxylate (Intermediate 96, 1.20 g, 79%) as an off white solid, which was used for the next step without further purification. ¹H NMR (400 MHz, DMSO-d₆) δ ppm 1.42 (s, 9H), 1.49-1.58 (m, 2H), 1.70-1.80 (m, 2H), 2.66-2.90 (m, 3H), 4.05-4.10 (m, 2H), 7.31 (br s, 1H), 7.35-7.41 (m, 1H), 7.52-7.65 (m, 1H), 7.69-7.72 (m, 1H), 7.75 (s, 1H), 7.95 (br s, 1H).

### Step 3

### Intermediate 97: tert-butyl (R)-6-(3-(1-(tert-butoxycarbonyl) piperidin-4-yl)benzamido)-2-(1-methylpyrrolidin-2-yl)-1H-pyrrolo[3,2-c]pyridine-1-carboxylate

To a solution of tert-butyl 4-(3-carbamoylphenyl) piperidine-1-carboxylate (Intermediate 96, 0.32 g, 1.05 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 0.50 g, 1.31 mmol) followed by cesium carbonate (1.29 g, 3.94 mmol) at room temperature and the reaction mixture was purged with argon for 15 min. Pd₂(dba)₃ (0.18 g, 0.19 mmol) and Xantphos (0.23 g, 0.39 mmol) were added and the reaction mixture was further purged with argon for 10 min and then heated at 100° C. for 2 h. The reaction mixture was concentrated in vacuo to obtain crude residue which was purified by combi-flash chromatography, by eluting with 80% ethyl acetate in heptane, to afford tert-butyl (R)-6-(3-(1-(tert-butoxycarbonyl) piperidin-4-yl)benzamido)-2-(1-methylpyrrolidin-2-yl)-1H-pyrrolo[3,2-c]pyridine-1-carboxylate (Intermediate 97, 0.38 g, 48%) as a yellow solid. ¹H NMR (400 MHz, DMSO-d₆) δ ppm 1.42 (s, 9H), 1.50-1.56 (m, 1H), 1.56-1.64 (m, 1H), 1.69 (s, 9H), 1.75-1.84 (m, 2H), 2.35 (s, 3H), 2.37-2.42 (m, 2H), 2.71-2.90 (m, 4H), 3.11-3.16 (m, 2H), 3.88-3.96 (m, 2H), 4.03-4.15 (m, 2H), 6.75 (s, 1H), 7.39-7.47 (m, 2H), 7.84-7.86 (m, 1H), 7.98 (s, 1H), 8.59 (s, 1H), 8.93 (s, 1H), 10.76 (br s, 1H). Mass spec: m/z: 604.49 [M+H]⁺.

### Step 4

### Intermediate 98: (R)—N-(2-(1-methylpyrrolidin-2-yl)-1H-pyrrolo[3,2-c]pyridin-6-yl)-3-(piperidin-4-yl)benzamide dihydrochloride

To a solution of tert-butyl (R)-6-(3-(1-(tert-butoxycarbonyl)piperidin-4-yl)benzamido)-2-(1-methylpyrrolidin-2-yl)-1H-pyrrolo[3,2-c]pyridine-1-carboxylate (Intermediate 97, 0.35 g, 0.58 mmol) in 1,4-dioxane (5 mL) was added 4M HCl in 1,4-dioxane (10 mL) at 0° C. and the reaction mixture was stirred at room temperature for 16 h. The reaction mixture was concentrated in vacuo to afford as (R)—N-(2-(1-methylpyrrolidin-2-yl)-1H-pyrrolo[3,2-c]pyridin-6-yl)-3-(piperidin-4-yl)benzamide dihydrochloride (Intermediate 98, 0.35 g) as a brown solid, which was used for the next step without further purification. Mass spec: m/z: 404.32 [M+H]⁺.

### Step 5

### Example 14: 3-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)benzamide

To a solution of (R)—N-(2-(1-methylpyrrolidin-2-yl)-1H-pyrrolo[3,2-c]pyridin-6-yl)-3-(piperidin-4-yl)benzamide dihydrochloride (Intermediate 98, 0.35 g, 0.86 mmol) in dimethyl sulfoxide (3 mL) was added 2-(2,6-dioxopiperidin-3-yl)-4-fluoroisoindoline-1,3-dione (CAS No: 835616-60-9, 0.24 g, 0.86 mmol) followed by N,N-diisopropylethylamine (0.76 mL, 4.33 mmol) and the reaction mixture was heated at 130° C. for 2 h. The reaction mixture was then poured into water (10 mL), resulting in a precipitate, which was filtered and dried to obtain crude compound. The crude material was purified by preparative HPLC (Method A) to afford 3-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)benzamide (Example 14, 0.19 g, 34%) as a yellow solid. ¹H NMR (400 MHz, DMSO-d₆) δ ppm 1.76-2.09 (m, 8H), 2.12-2.15 (m, 1H), 2.17 (s, 3H), 2.23-2.30 (m, 1H), 2.56-2.63 (m, 2H), 2.81-2.90 (m, 2H), 3.01-3.10 (m, 2H), 3.11-3.19 (m, 1H), 3.32-3.36 (m, 1H), 3.84-3.90 (m, 2H), 5.08-5.14 (m, 1H), 6.39 (s, 1H), 7.36 (d, J=7.20 Hz, 1H), 7.39-7.53 (m, 3H), 7.70-7.74 (m, 1H), 7.88 (d, J=7.23 Hz, 1H), 8.04 (s, 1H), 8.21 (s, 1H), 8.50 (s, 1H), 10.57 (s, 1H), 11.07 (s, 1H), 11.36 (s, 1H). Mass spec: m/z: 660.0 [M+H]⁺.

### Example 15 was Synthesised Following Scheme 18

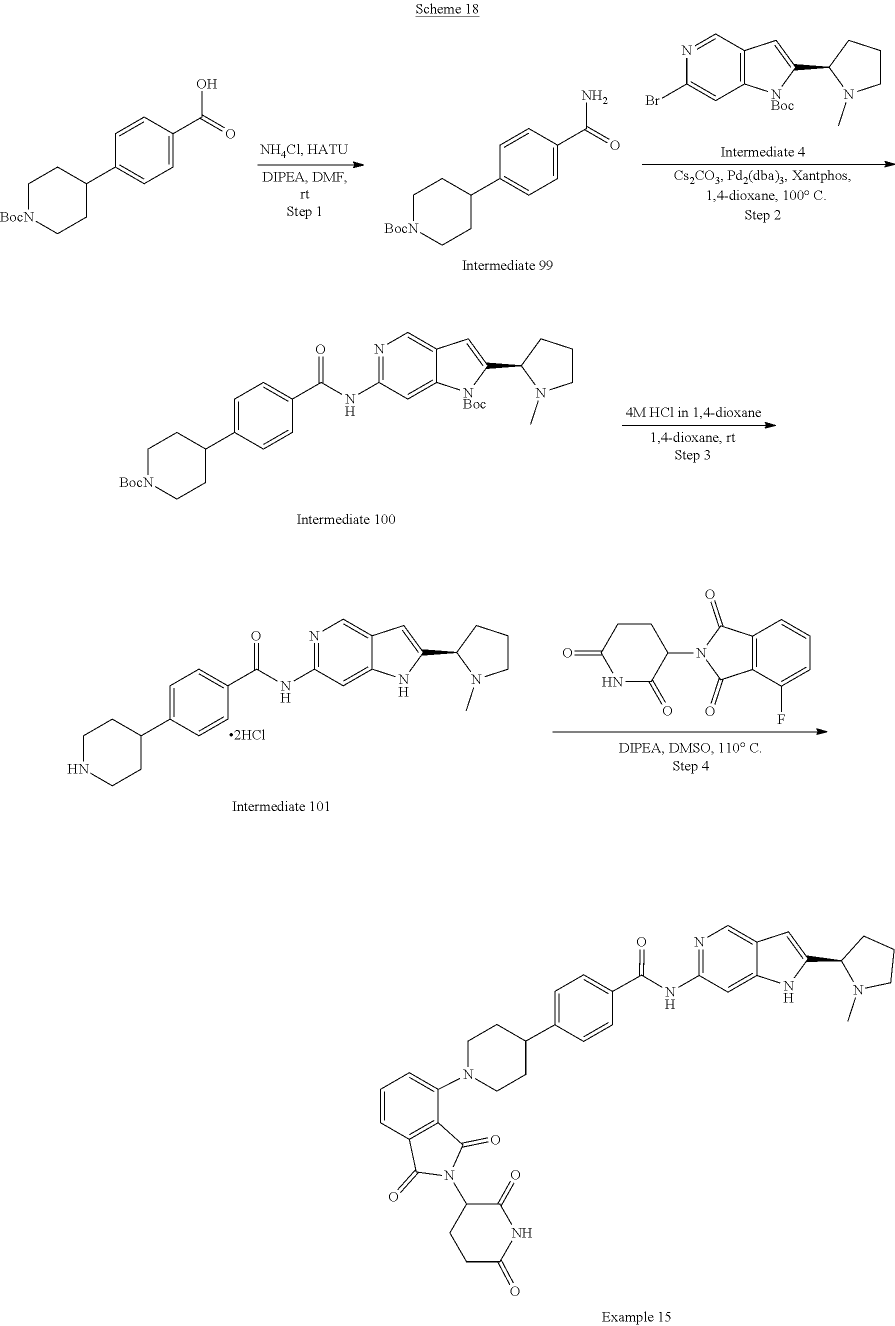

### Step 1

### Intermediate 99: tert-butyl 4-(4-carbamoylphenyl) piperidine-1-carboxylate

To a solution of 4-(1-(tert-butoxycarbonyl)piperidin-4-yl)benzoic acid (CAS No: 149353-75-3, 1.5 g, 4.9 mmol) in dimethylformamide (30 mL) was added HATU (2.9 g, 7.4 mmol) and N,N-diisopropylethylamine (3.4 mL, 20 mmol) at room temperature and stirred for 10 min. Ammonium chloride (1.1 g, 20 mmol) was added at 0° C. and the reaction mixture was stirred at room temperature for 16 h. The reaction mixture was poured into water (100 mL) to obtain the solid precipitate which was filtered and dried under vacuum to afford tert-butyl 4-(4-carbamoylphenyl) piperidine-1-carboxylate (Intermediate 99, 1.30 g, 87%) as a white solid, which was used in the next step without further purification. Mass spec: m/z 205.08 [M-Boc]+.

### Step 2

### Intermediate 100: tert-butyl (R)-6-(4-(1-(tert-butoxycarbonyl)piperidin-4-yl)benzamido)-2-(1-methylpyrrolidin-2-yl)-1H-pyrrolo[3,2-c]pyridine-1-carboxylate

To a solution of tert-butyl 4-(4-carbamoylphenyl)piperidine-1-carboxylate (Intermediate 99, 0.32 g, 1.05 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 0.50 g, 1.31 mmol) followed by cesium carbonate (0.86 g, 2.63 mmol) at room temperature and the mixture was purged with argon for 15 min. Pd₂(dba)₃ (0.18 g, 0.19 mmol) and Xantphos (0.23 g, 0.39 mmol) were added and the reaction mixture was purged with argon for a further 10 min and then heated at 100° C. for 2 h. The reaction mixture was concentrated in vacuo to obtain the crude compound which was then was purified by combi-flash column chromatography, by eluting with 80% ethyl acetate in heptane, to afford tert-butyl (R)-6-(4-(1-(tert-butoxycarbonyl)piperidin-4-yl)benzamido)-2-(1-methylpyrrolidin-2-yl)-1H-pyrrolo[3,2-c]pyridine-1-carboxylate (Intermediate 100, 0.51 g, 64%) as a yellow solid. ¹H NMR (400 MHz, DMSO-d₆) δ ppm 1.42 (s, 9H), 1.50-1.56 (m, 1H), 1.56-1.64 (m, 1H), 1.69 (s, 9H), 1.75-1.84 (m, 2H), 2.35 (s, 3H), 2.37-2.42 (m, 2H), 2.71-2.90 (m, 4H), 3.11-3.16 (m, 2H), 3.88-3.96 (m, 2H), 4.03-4.15 (m, 2H), 6.75 (s, 1H), 7.38 (d, J=7.88 Hz, 2H), 7.98 (d, J=7.88 Hz, 2H), 8.58 (s, 1H), 8.92 (s, 1H), 10.61 (s, 1H). Mass spec: m/z: 604.57 [M+H]⁺.

### Step 3

### Intermediate 101: (R)—N-(2-(1-methylpyrrolidin-2-yl)-1H-pyrrolo[3,2-c]pyridin-6-yl)-4-(piperidin-4-yl)benzamide dihydrochloride

To a solution of tert-butyl (R)-6-(4-(1-(tert-butoxycarbonyl)piperidin-4-yl)benzamido)-2-(1-methylpyrrolidin-2-yl)-1H-pyrrolo[3,2-c]pyridine-1-carboxylate (Intermediate 100, 0.4 g, 0.66 mmol) in 1,4-dioxane (3 mL) was added 4M HCl in 1,4-dioxane (5 mL) at 0° C. and the reaction mixture was stirred at room temperature for 16 h. The reaction mixture was concentrated in vacuo to afford (R)—N-(2-(1-methylpyrrolidin-2-yl)-1H-pyrrolo[3,2-c]pyridin-6-yl)-4-(piperidin-4-yl)benzamide dihydrochloride (Intermediate 101, 0.3 g) as an off white solid, which was used for the next step without further purification. ¹H NMR (400 MHz, DMSO-d₆) δ ppm 1.86-2.02 (m, 3H), 2.12-2.26 (m, 2H), 2.33-2.44 (m, 1H), 2.53 (s, 3H), 2.74-2.86 (m, 2H), 2.90-3.08 (m, 2H), 3.22-3.26 (m, 1H), 3.34-3.50 (m, 3H), 3.68-3.80 (m, 2H), 7.26 (s, 1H), 7.46 (d, J=7.88 Hz, 2H), 8.18 (d, J=7.88 Hz, 1H), 8.27 (s, 1H), 8.92-9.08 (m, 2H), 11.41 (br s, 1H), 11.94 (br s, 1H), 13.20 (br s, 1H). Mass spec: m/z: 404.34 [M+H]⁺.

### Step 4

### Example 15: 4-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)benzamide

To a solution of of (R)—N-(2-(1-methylpyrrolidin-2-yl)-1H-pyrrolo[3,2-c]pyridin-6-yl)-4-(piperidin-4-yl)benzamide dihydrochloride (Intermediate 101, 0.30 g, 0.74 mmol) in dimethyl sulfoxide (2 mL) was added 2-(2,6-dioxopiperidin-3-yl)-4-fluoroisoindoline-1,3-dione (CAS No: 835616-60-9, 0.20 g, 0.74 mmol) followed by DIPEA (0.65 mL, 3.72 mmol) and the reaction mixture was heated at 110° C. for 2 h. The reaction mixture was poured into water (10 mL), resulting in a precipitate which was filtered and dried to obtain crude compound. The crude material was purified by preparative HPLC (Method A) to afford 4-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)benzamide (Example 15, 0.15 g, 31%) as a yellow solid. ¹H NMR (400 MHz, DMSO-d₆) δ ppm 1.76-1.98 (m, 8H), 2.01-2.08 (m, 1H), 2.12-2.14 (m, 1H), 2.17 (s, 3H), 2.22-2.30 (m, 1H), 2.55-2.64 (m, 2H), 2.80-2.94 (m, 2H), 2.99-3.09 (m, 2H), 3.09-3.17 (m, 1H), 3.82-3.88 (m, 2H), 5.08-5.16 (m, 1H), 6.39 (s, 1H), 7.35-7.45 (m, 4H), 7.70-7.74 (m, 1H), 8.01-8.04 (m, 2H), 8.20 (s, 1H), 8.50 (s, 1H), 10.39 (s, 1H), 11.09 (s, 1H), 11.36 (s, 1H). Mass spec: m/z: 660.05[M+H]⁺.

### Example 16 was Synthesised Following Scheme 19

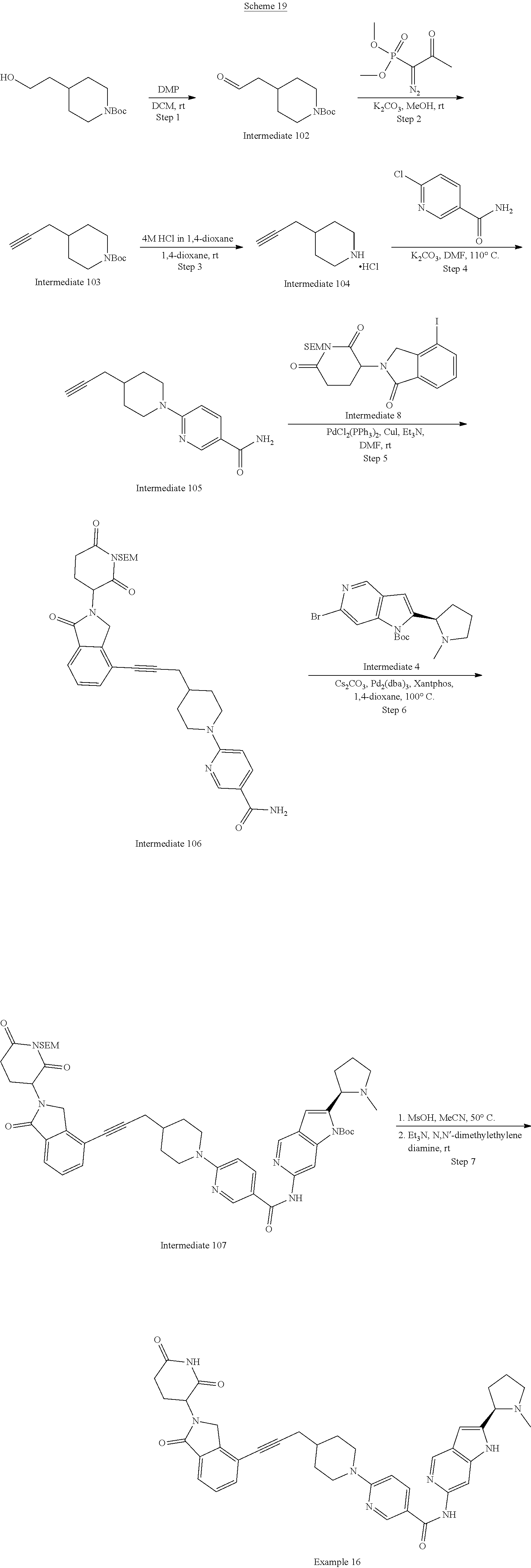

### Step 1

### Intermediate 102: tert-butyl 4-(2-oxoethyl) piperidine-1-carboxylate

To a solution of tert-butyl 4-(2-hydroxyethyl)piperidine-1-carboxylate (CAS No: 89151-44-0, 10.0 g, 43.6 mmol) in dichloromethane (150 mL) was added Dess-Martin Periodinane (27.7 g, 65.4 mmol) at 0° C. and the reaction mixture was stirred at room temperature for 16 h under a nitrogen atmosphere. The reaction mixture was quenched with saturated NH₄Cl solution (10 mL) and extracted with dichloromethane (3×200 mL). The combined organic layers were dried over anhydrous Na₂SO₄, filtered, and concentrated in vacuo to afford tert-butyl 4-(2-oxoethyl) piperidine-1-carboxylate (Intermediate 102, 7.0 g) as colourless oil, which was used for the next step without further purification. ¹H NMR (400 MHz, DMSO-d₆) δ ppm 0.92-1.10 (m, 2H), 1.35 (s, 9H), 1.52-1.62 (m, 2H), 1.90-2.00 (m, 1H), 2.30-2.38 (m, 2H), 2.62-2.78 (m, 2H), 3.82-3.96 (m, 2H), 9.63 (s, 1H).

### Step 2

### Intermediate 103: tert-butyl 4-(prop-2-yn-1-yl) piperidine-1-carboxylate

To a solution of tert-butyl 4-(2-oxoethyl) piperidine-1-carboxylate (Intermediate 102, 6.50 g, 29.0 mmol) in methanol (80 mL) was added potassium carbonate (5.9 g, 43.0 mmol) at 0° C. followed by dimethyl (1-diazo-2-oxopropyl)phosphonate (5.5 g, 29.0 mmol) and the reaction mixture was stirred at 0° C. for 3 h. The reaction mixture was diluted with water (500 mL) and extracted with ethyl acetate (3×200 mL). The combined organic phases were dried over anhydrous Na₂SO₄, filtered, and concentrated in vacuo. The crude material was purified by combi-flash column chromatography, by eluting with 80% ethyl acetate in heptane, to afford tert-butyl 4-(prop-2-yn-1-yl) piperidine-1-carboxylate (Intermediate 103, 4.5 g, 70%) as a yellow liquid. ¹H NMR (400 MHz, DMSO-d₆) δ ppm 0.96-1.11 (m, 2H), 1.36 (s, 9H), 1.47-1.71 (m, 3H), 2.02-2.18 (m, 2H), 2.56-2.72 (m, 2H), 2.77 (s, 1H), 3.81-4.00 (m, 2H).

### Step 3

### Intermediate 104: 4-(prop-2-yn-1-yl)piperidine hydrochloride

To a solution of tert-butyl 4-(prop-2-yn-1-yl)piperidine-1-carboxylate (Intermediate 103, 4.5 g, 20.0 mmol) in 1,4-dioxane (10 mL) was added 4M HCl in 1,4-dioxane (40 mL) at 0° C. and the reaction mixture was stirred at room temperature for 16 h. The reaction mixture was concentrated in vacuo to afford 4-(prop-2-yn-1-yl)piperidine hydrochloride (Intermediate 104, 4.0 g) as brown solid, which was used for the next step without further purification. ¹H NMR (400 MHz, DMSO-d₆) δ ppm 1.33-1.48 (m, 2H), 1.43-71 (m, 1H), 1.79-1.85 (m, 2H), 2.15-2.18 (m, 2H), 2.82 (t, J=11.6 Hz, 2H), 2.89 (s, 1H), 3.21-3.25 (m, 2H), 8.62 (br s, 1H), 8.89 (br s, 1H).

### Step 4

### Intermediate 105: 6-(4-(prop-2-yn-1-yl) piperidin-1-yl)nicotinamide

To a solution of 4-(prop-2-yn-1-yl)piperidine (Intermediate 104, 2.50 g, 20.0 mmol) and 6-chloronicotinamide (CAS No: 6271-78-9, 3.2 g, 20.0 mmol) in dimethylformamide (25 mL) was added potassium carbonate (11.0 g, 81.0 mmol) and the reaction mixture was heated at 110° C. for 16 h. The reaction mixture was poured into ice cold water (200 mL), resulting in a precipitate, which was filtered and dried in vacuo to afford 6-(4-(prop-2-yn-1-yl) piperidin-1-yl) nicotinamide (Intermediate 105, 1.70 g, 34%) as an off white solid, which was used in the next step without further purification. ¹H NMR (400 MHz, DMSO-d₆) δ ppm 1.11-1.25 (m, 2H), 1.69-1.83 (m, 3H), 2.14-2.16 (m, 2H), 2.76-2.85 (m, 2H), 2.85 (s, 1H), 4.39-4.44 (m, 2H), 6.82 (d, J=9.20 Hz, 1H), 7.08 (br s, 1H), 7.71 (br s, 1H), 7.92 (d, J=8.80 Hz, 1H), 8.59 (s, 1H). Mass spec: m/z: 244.12 [M+H]⁺.

### Step 5

### Intermediate 106: 6-(4-(3-(2-(2,6-dioxo-1-((2-(trimethylsilyl)ethoxy)methyl)piperidin-3-yl)-1-oxoisoindolin-4-yl) prop-2-yn-1-yl) piperidin-1-yl) nicotinamide

To a solution of 6-(4-(prop-2-yn-1-yl)piperidin-1-yl)nicotinamide (Intermediate 105, 0.5 g, 2.05 mmol) in dimethylformamide (5 mL) was added 3-(4-iodo-1-oxoisoindolin-2-yl)-1-((2-(trimethylsilyl)ethoxy)methyl)piperidine-2,6-dione (Intermediate 8, 1.02 g, 2.05 mmol) followed by triethylamine (10.7 mL, 76.04 mmol) and the reaction mixture was purged with argon gas for 15 min. PdCl₂(PPh₃)₂ (0.14 g, 0.20 mmol) and copper(I) iodide (0.03 g, 0.20 mmol) were added and the reaction mixture was further purged with argon gas for 10 min and stirred at room temperature for 3 h. The reaction mixture was then poured into water (60 mL) and extracted with ethyl acetate (3×100 mL). The combined organic phases were dried over anhydrous Na₂SO₄, filtered, and concentrated in vacuo. The crude material was purified by combi-flash column chromatography, by eluting with 100% ethyl acetate, to afford 6-(4-(3-(2-(2,6-dioxo-1-((2-(trimethylsilyl)ethoxy)methyl) piperidin-3-yl)-1-oxoisoindolin-4-yl) prop-2-yn-1-yl) piperidin-1-yl)nicotinamide (Intermediate 106, 0.75 g, 59%) as a yellow solid. ¹H NMR (400 MHz, DMSO-d₆) δ ppm −0.03 (s, 9H), 0.79-0.89 (m, 2H), 1.21-1.33 (m, 2H), 1.80-1.93 (m, 3H), 2.01-2.10 (m, 1H), 2.35-2.47 (m, 2H), 2.75-2.94 (m, 3H), 2.98-3.13 (m, 2H), 3.48-3.58 (m, 2H), 4.25-4.29 (m, 1H), 4.39-4.50 (m, 3H), 4.98-5.13 (m, 2H), 5.23-5.27 (m, 1H), 6.82 (d, J=9.20 Hz, 1H), 7.08 (br s, 1H), 7.52 (t, J=7.60 Hz, 1H), 7.63 (d, J=7.20 Hz, 1H), 7.71 (d, J=7.20 Hz, 2H), 7.91-7.94 (m, 1H), 8.59 (s, 1H). Mass spec: m/z: 616.41 [M+H]⁺.

### Step 6

### Intermediate 107: tert-butyl 6-(6-(4-(3-(2-(2,6-dioxo-1-((2-(trimethylsilyl)ethoxy)methyl)piperidin-3-yl)-1-oxoisoindolin-4-yl)prop-2-yn-1-yl)piperidin-1-yl)nicotinamido)-2-((R)-1-methylpyrrolidin-2-yl)-1H-pyrrolo[3,2-c]pyridine-1-carboxylate

To a solution of 6-(4-(3-(2-(2,6-dioxo-1-((2-(trimethylsilyl) ethoxy)methyl) piperidin-3-yl)-1-oxoisoindolin-4-yl) prop-2-yn-1-yl) piperidin-1-yl)nicotinamide (Intermediate 106, 0.51 g, 0.84 mmol)) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 0.4 g, 1.05 mmol) followed by cesium carbonate (0.68 g, 2.10 mmol) at room temperature and the reaction mixture was purged with argon for 15 min. Pd₂(dba)₃ (0.14 g, 0.15 mmol) and Xantphos (0.18 g, 0.31 mmol) were added and the reaction mixture was purged with argon for 10 min and then heated at 100° C. for 2 h. The reaction mixture was concentrated in vacuo and the crude material was purified by combi-flash column chromatography, by eluting with 80% ethyl acetate in heptane, to afford tert-butyl 6-(6-(4-(3-(2-(2,6-dioxo-1-((2-(trimethylsilyl)ethoxy)methyl)piperidin-3-yl)-1-oxoisoindolin-4-yl)prop-2-yn-1-yl)piperidin-1-yl)nicotinamido)-2-((R)-1-methylpyrrolidin-2-yl)-1H-pyrrolo[3,2-c]pyridine-1-carboxylate (Intermediate 107, 0.41 g, 43%) as a yellow solid. ¹H NMR (400 MHz, DMSO-d₆) δ ppm −0.03 (s, 9H), 0.78-0.88 (m, 2H), 1.23-1.35 (m, 2H), 1.54-1.64 (m, 1H), 1.69 (s, 9H), 1.72-1.80 (m, 2H), 1.86-1.92 (m, 4H), 2.01-2.10 (m, 1H), 2.28-2.32 (m, 1H), 2.35 (s, 3H), 2.38-2.45 (m, 2H), 2.76-2.82 (m, 1H), 2.90-2.96 (m, 2H), 3.00-3.19 (m, 3H), 3.46-3.59 (m, 2H), 3.89-3.92 (m, 1H), 4.26-4.30 (m, 1H), 4.41-4.55 (m, 3H), 5.01-5.10 (m, 2H), 5.23-5.28 (m, 1H), 6.74 (s, 1H), 6.88 (d, J=9.20 Hz, 1H), 7.49-7.57 (m, 1H), 7.65 (d, J=7.89 Hz, 1H), 7.72 (d, J=7.89 Hz, 1H), 8.14 (d, J=7.45 Hz, 1H), 8.57 (s, 1H), 8.79 (s, 1H), 8.91 (s, 1H), 10.47 (s, 1H). Mass spec: m/z: 815.5 [M+H]⁺.

### Step 7

### Example 16: 6-(4-(3-(2-(2,6-dioxopiperidin-3-yl)-1-oxoisoindolin-4-yl)prop-2-yn-1-yl)piperidin-1-yl)-N-(2-((R)-1-methylpyrrolidin-2-yl)-1H-pyrrolo[3,2-c]pyridin-6-yl)nicotinamide

To a solution of tert-butyl 6-(6-(4-(3-(2-(2,6-dioxo-1-((2-(trimethylsilyl)ethoxy)methyl)piperidin-3-yl)-1-oxoisoindolin-4-yl)prop-2-yn-1-yl)piperidin-1-yl)nicotinamido)-2-((R)-1-methylpyrrolidin-2-yl)-1H-pyrrolo[3,2-c]pyridine-1-carboxylate (Intermediate 107, 0.40 g, 0.43 mmol) in acetonitrile (10 mL) was added methanesulfonic acid (0.43 mL, 6.55 mmol) at 0° C. and the reaction heated at 50° C. for 2 h. N,N′-dimethylethylenediamine (0.31 mL, 2.62 mmol) and triethylamine (0.88 g, 8.74 mmol) were added at room temperature and the reaction stirred for 2 h. The reaction mixture was concentrated in vacuo water (100 mL) was added, resulting in a precipitate which was filtered and dried in vacuo. The crude material was purified by preparative HPLC (Method A) to afford 6-(4-(3-(2-(2,6-dioxopiperidin-3-yl)-1-oxoisoindolin-4-yl)prop-2-yn-1-yl)piperidin-1-yl)-N-(2-((R)-1-methylpyrrolidin-2-yl)-1H-pyrrolo[3,2-c]pyridin-6-yl)nicotinamide (Example 16, 0.11 g, 37%). ¹H NMR (400 MHz, DMSO-d₆) δ ppm 1.22-1.36 (m, 2H), 1.76-1.96 (m, 6H), 1.97-2.06 (m, 1H), 2.10-2.14 (m, 1H), 2.16 (s, 3H), 2.23-2.29 (m, 1H), 2.40-2.45 (m, 1H), 2.58-2.62 (m, 2H), 2.85-2.99 (m, 3H), 3.08-3.18 (m, 1H), 3.26-3.30 (m, 1H), 3.34-3.36 (m, 1H), 4.27-4.37 (m, 1H), 4.40-4.55 (m, 3H), 5.10-5.15 (m, 1H), 6.37 (s, 1H), 6.88 (d, J=9.13 Hz, 1H), 7.48-7.55 (m, 1H), 7.64-7.79 (m, 2H), 8.12-8.17 (m, 2H), 8.48 (s, 1H), 8.79 (m, 1H), 10.23 (s, 1H), 11.00 (s, 1H), 11.32 (s, 1H). Mass spec: m/z: 685.05 [M+H]⁺.

### Example 17 was Synthesised Following Scheme 20

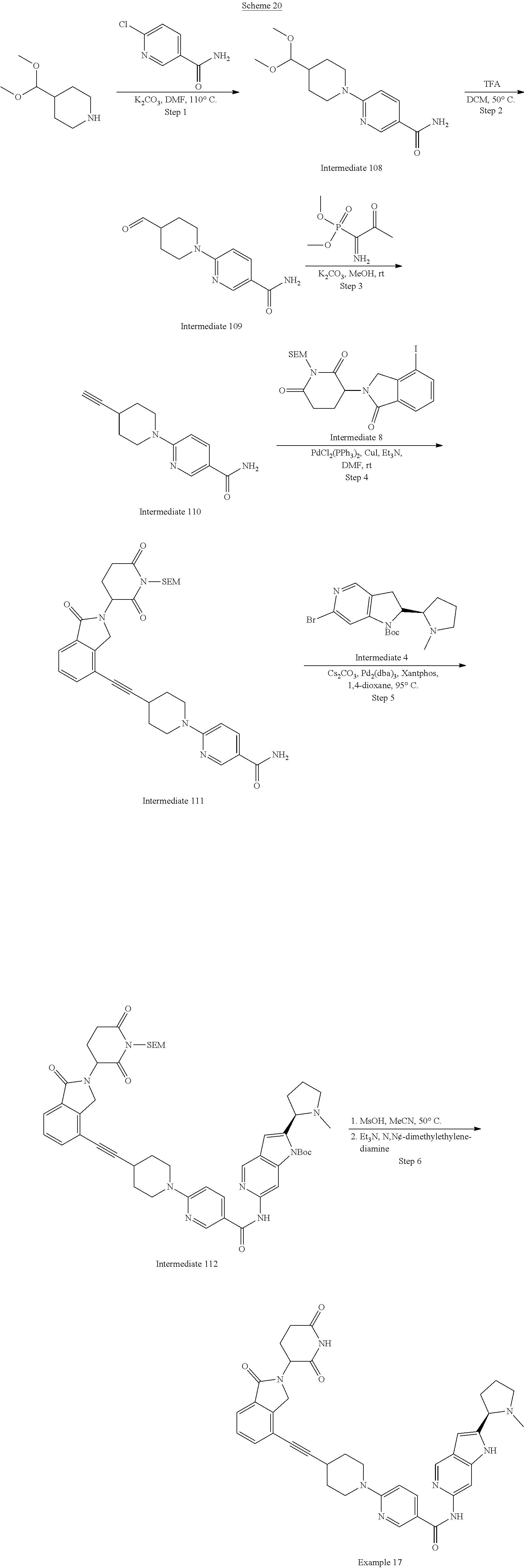

### Step 1

### Intermediate 108: 6-(4-(dimethoxymethyl)piperidin-1-yl)nicotinamide

To a solution of 6-chloronicotinamide (CAS No: 6271-78-9, 4.00 g, 25.5 mmol) in dimethylformamide (50 mL) were added potassium carbonate (10.6 g, 76.6 mmol) and 4-(dimethoxymethyl)piperidine (CAS No: 188646-83-5, 4.47 g, 28.1 mmol) at room temperature and the reaction mixture was heated at 110° C. for 16 h. The reaction mixture was poured into ice cold water (400 mL), a solid precipitated, which was filtered and dried in vacuo to afford 6-(4-(dimethoxymethyl)piperidin-1-yl)nicotinamide (Intermediate 108, 5.5 g, 77%) as an off white solid, which was used for the next step without further purification. ¹H NMR (400 MHz, DMSO-d₆) δ ppm 1.10-1.23 (m, 2H), 1.66-1.72 (m, 2H), 1.81-1.93 (m, 1H), 2.79-2.85 (m, 2H), 3.26 (s, 6H), 4.01-4.10 (m, 1H), 4.38-4.44 (m, 2H), 6.81 (d, J=9.12 Hz, 1H), 7.08 (br s, 1H), 7.71 (br s, 1H), 7.92 (d, J=9.12 Hz, 1H), 8.58 (s, 1H). Mass spec: m/z 280.19 [M+H]⁺.

### Step 2

### Intermediate 109: 6-(4-formylpiperidin-1-yl)nicotinamide

To a solution of 6-(4-(dimethoxymethyl)piperidin-1-yl)nicotinamide (Intermediate 108, 5.5 g, 20.0 mmol) in dichloromethane (60 mL) was added trifluoroacetic acid (15 mL) at 0° C. and the reaction mixture was heated at 50° C. for 2 h. the reaction mixture was basified with saturated NaHCO₃ (200 mL) solution to pH~8 and extracted with ethyl acetate (3×150 mL). The combined organic layers were dried over anhydrous Na₂SO₄, and filtered and concentrated in vacuo to afford 6-(4-formylpiperidin-1-yl)nicotinamide (Intermediate 109, 4.5 g, 98%) as a gum, which was used for the next step without further purification. ¹H NMR (400 MHz, DMSO-d₆) δ ppm 1.40-1.54 (m, 2H), 1.84-1.93 (m, 2H), 2.58-2.67 (m, 1H), 3.10-3.16 (m, 2H), 4.20-4.26 (m, 2H), 6.85 (d, J=8.77 Hz, 1H), 7.10 (br s, 1H), 7.73 (br s, 1H), 7.93 (d, J=8.77, 1H), 8.60 (s, 1H), 9.61 (s, 1H). Mass spec: m/z: 234.4 [M+H]⁺.

### Step 3

### Intermediate 110: 6-(4-ethynylpiperidin-1-yl) nicotinamide

To a solution of 6-(4-formylpiperidin-1-yl)nicotinamide (Intermediate 109, 4.5 g, 19.0 mmol) in methanol (60 mL) was added potassium carbonate (5.6 g, 39.0 mmol) at 0° C. followed by dimethyl (1-diazo-2-oxopropyl)phosphonate (5.6 g, 29.0 mmol) and the reaction mixture was stirred at room temperature for 2 h. The reaction mixture was diluted with water (120 mL) and extracted with ethyl acetate (3×150 mL). The combined organic layers were separated, dried over anhydrous Na₂SO₄, filtered, and concentrated in vacuo. The crude material was purified by combi-flash column chromatography, by eluting with 8% MeOH in DCM, to afford 6-(4-ethynylpiperidin-1-yl)nicotinamide (Intermediate 110, 3.0 g, 68%) as an off white solid. ¹H NMR (400 MHz, DMSO-d₆) δ ppm 1.43-1.56 (m, 2H), 1.78-1.84 (m, 2H), 2.65-2.74 (m, 1H), 2.95 (s, 1H), 3.32-3.36 (m, 2H), 3.92-4.04 (m, 2H), 6.84 (d, J=9.21 Hz, 1H), 7.10 (br s, 1H), 7.73 (br s, 1H), 7.93 (d, J=7.89 Hz, 1H), 8.60 (s, 1H). Mass spec: m/z: 230.11 [M+H]⁺.

### Step 4

### Intermediate 111: 6-(4-((2-(2,6-dioxo-1-((2-(trimethylsilyl) ethoxy)methyl) piperidin-3-yl)-1-oxoisoindolin-4-yl) ethynyl) piperidin-1-yl)nicotinamide

To a solution of 6-(4-ethynylpiperidin-1-yl)nicotinamide (Intermediate 110, 0.22 g, 0.99 mmol) and 3-(4-iodo-1-oxoisoindolin-2-yl)-1-((2-(trimethylsilyl)ethoxy)methyl)piperidine-2,6-dione (Intermediate 8, 0.5 g, 0.99 mmol) in dimethylformamide (2 mL) was added triethylamine (6 mL, 36.97 mmol) and the reaction mixture was purged with argon gas for 15 min. Pd(PPh₃)₂Cl₂ (0.11 g, 0.14 mmol) and copper (I) iodide (0.02 g, 0.09 mmol) were added and the reaction mixture was further purged with argon gas for 10 min and stirred at room temperature for 1 h. The reaction mixture was diluted with water (50 mL) and extracted with ethyl acetate (2×100 mL). The combined organic layers were dried over anhydrous Na₂SO₄, filtered, and concentrated in vacuo. The crude material was purified by combi-flash column chromatography, by eluting with 8% MeOH in DCM, to afford 6-(4-((2-(2,6-dioxo-1-((2-(trimethylsilyl) ethoxy)methyl) piperidin-3-yl)-1-oxoisoindolin-4-yl) ethynyl) piperidin-1-yl) nicotinamide (Intermediate 111, 0.30 g, 50%) as a yellow solid. ¹H NMR (400 MHz, DMSO-d₆) δ ppm −0.04 (s, 9H), 0.77-0.89 (m, 2H), 1.15-1.19 (m, 4H), 1.61-1.66 (m, 2H), 2.01-2.10 (m, 2H), 2.76-2.82 (m, 1H), 2.97-3.08 (m, 2H), 3.37-3.45 (m, 2H), 3.46-3.58 (m, 2H), 4.25-4.29 (m, 1H), 4.44-4.50 (m, 1H), 5.00-5.10 (m, 2H), 5.23-5.28 (m, 1H), 6.86 (d, J=8.77 Hz, 1H), 7.10 (br s, 1H), 7.50-7.57 (m, 1H), 7.65-7.67 (m, 1H), 7.71-7.73 (m, 1H), 7.94 (d, J=8.33 Hz, 1H), 8.60 (br s, 1H). Mass spec: m/z: 602.36 [M+H]⁺.

### Step 5

### Intermediate 112: tert-butyl 6-(6-(4-((2-(2,6-dioxo-1-((2-(trimethylsilyl)ethoxy)methyl)piperidin-3-yl)-1-oxoisoindolin-4-yl)ethynyl)piperidin-1-yl)nicotinamido)-2-((R)-1-methylpyrrolidin-2-yl)-1H-pyrrolo[3,2-c]pyridine-1-carboxylate

To a mixture of 6-(4-((2-(2,6-dioxo-1-((2-(trimethylsilyl)ethoxy)methyl) piperidin-3-yl)-1-oxoisoindolin-4-yl) ethynyl)piperidin-1-yl)nicotinamide (Intermediate 111, 0.22 g, 0.57 mmol) and tert-butyl (R)-6-bromo-2-(1-methylpyrrolidin-2-yl)-1H-pyrrolo[3,2-c]pyridine-1-carboxylate (Intermediate 4, 0.27 g, 0.46 mmol) in 1,4-dioxane (3 mL) was added cesium carbonate (0.29 g, 0.86 mmol) at room temperature and the reaction mixture was purged with argon for 10 min followed. Pd₂(dba)₃ (0.07 g, 0.08 mmol) and Xantphos (0.10 g, 0.17 mmol) were added and the reaction mixture was further purged with argon for 10 min and then heated at 95° C. for 1 h. The reaction mixture was concentrated in vacuo and the crude material was purified by combi-flash column chromatography, by eluting with 8% MeOH in DCM, to afford tert-butyl 6-(6-(4-((2-(2,6-dioxo-1-((2-(trimethylsilyl)ethoxy)methyl)piperidin-3-yl)-1-oxoisoindolin-4-yl)ethynyl)piperidin-1-yl)nicotinamido)-2-((R)-1-methylpyrrolidin-2-yl)-1H-pyrrolo[3,2-c]pyridine-1-carboxylate (Intermediate 112, 0.35 g, 67%) as a yellow solid. ¹H NMR (400 MHz, DMSO-d₆) δ ppm −0.04 (s, 9H), 0.77-0.88 (m, 2H), 1.16-1.20 (m, 1H), 1.58-1.66 (m, 4H), 1.69 (s, 9H), 1.73-1.79 (m, 2H), 1.93-1.99 (m, 2H), 2.02-2.10 (m, 1H), 2.30-2.33 (m, 1H), 2.35 (s, 3H), 2.75-2.83 (m, 1H), 3.00-3.17 (m, 3H), 3.41-3.57 (m, 4H), 3.89-3.94 (m, 1H), 4.03-4.14 (m, 2H), 4.26-4.30 (m, 1H), 4.46-4.52 (m, 1H), 4.99-5.10 (m, 2H), 5.24-5.28 (m, 1H), 6.74 (s, 1H), 6.91 (d, J=9.29 Hz, 1H), 7.50-7.56 (m, 1H), 7.66-7.75 (m, 2H), 8.14-8.17 (m, 1H), 8.57 (s, 1H), 8.80 (s, 1H), 8.90 (s, 1H), 10.49 (s, 1H). Mass spec: m/z: 901.55 [M+H]⁺.

### Step 6

### Example 17: 6-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)nicotinamide

To a solution of tert-butyl 6-(6-(4-((2-(2,6-dioxo-1-((2-(trimethylsilyl)ethoxy)methyl)piperidin-3-yl)-1-oxoisoindolin-4-yl)ethynyl)piperidin-1-yl)nicotinamido)-2-((R)-1-methylpyrrolidin-2-yl)-1H-pyrrolo[3,2-c]pyridine-1-carboxylate (Intermediate 112, 0.35 g, 0.38 mmol) in acetonitrile (3.5 mL) was added methanesulfonic acid (0.39 g, 3.88 mmol) at 0° C. and then heated at 50° C. for 2 h. N,N′-dimethylethylenediamine (0.21 g, 2.33 mmol) and triethylamine (0.82 g, 7.76 mmol) were then added and the reaction mixture was stirred at room temperature for 2 h. The reaction mixture was concentrated in vacuo and the resulting mixture was quenched with water (50 mL), affording a precipitate, which was filtered and dried in vacuo. The crude material was then purified by preparative HPLC (Method A) to afford 6-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)nicotinamide (Example 17, 0.11 g, 31%) as an off white solid. ¹H NMR (400 MHz, DMSO-d₆) δ ppm 1.61-1.73 (m, 2H), 1.75-1.92 (m, 3H), 1.93-2.05 (m, 3H), 2.11-2.14 (m, 1H), 2.16 (s, 3H), 2.21-2.30 (m, 1H), 2.43-2.47 (m, 1H), 2.56-2.63 (m, 1H), 2.84-2.96 (m, 1H), 3.03-3.09 (m, 1H), 3.11-3.19 (m, 1H), 3.24-3.28 (m, 1H), 3.40-3.52 (m, 2H), 4.04-4.15 (m, 2H), 4.28-4.38 (m, 1H), 4.40-4.51 (m, 1H), 5.10-5.15 (m, 1H), 6.38 (s, 1H), 6.91 (d, J=9.01 Hz, 1H), 7.48-7.56 (m, 1H), 7.63-7.75 (m, 2H), 8.13-8.20 (m, 2H), 8.48 (s, 1H), 8.80 (s, 1H), 10.26 (s, 1H), 10.97 (s, 1H), 11.33 (s, 1H). Mass spec: m/z: 671.05 [M+H]⁺.

### Example 18 was Synthesised Following Scheme 21

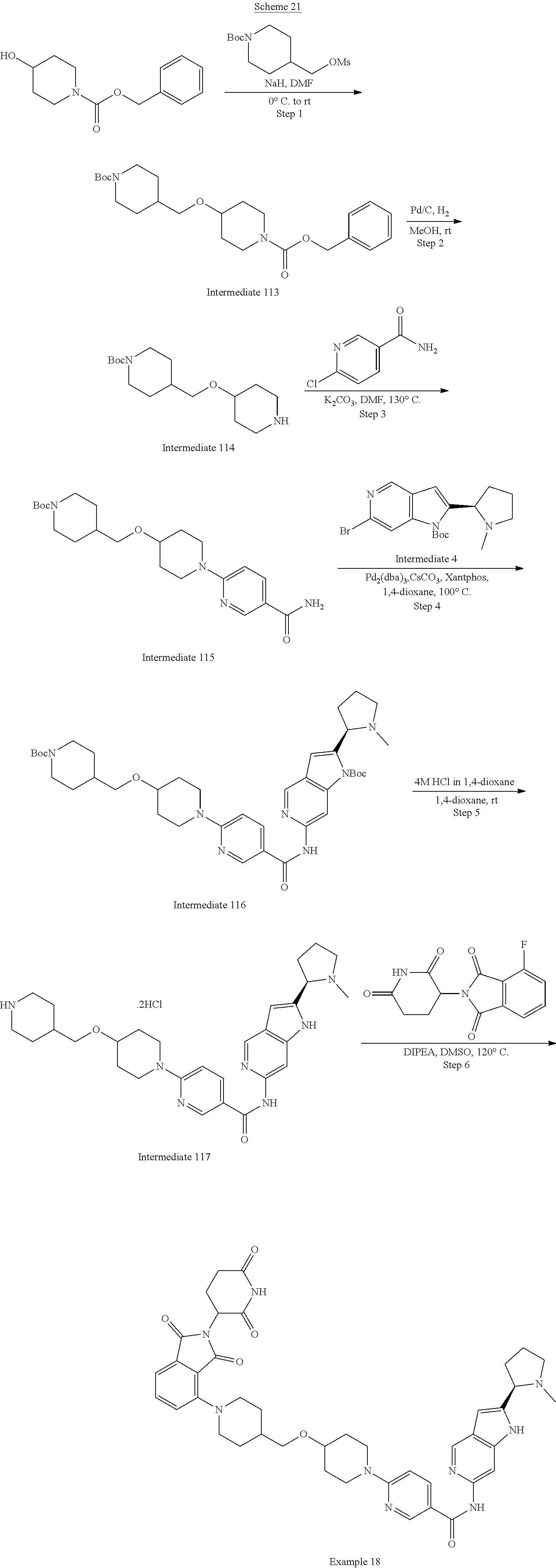

### Step 1

### Intermediate 113: benzyl 4-((1-(tert-butoxycarbonyl)piperidin-4-yl)methoxy)piperidine-1-carboxylate

To a solution of tert-butyl 4-(((methylsulfonyl)oxy)methyl)piperidine-1-carboxylate (CAS No: 161975-39-9, 17.0 g, 72.25 mmol) in dimethylformamide (210 mL) was added sodium hydride (4.33 g, 108.4 mmol) and the reaction stirred for 30 in at 0° C. Benzyl 4-hydroxypiperidine-1-carboxylate (CAS No: 95798-23-5, 23.32 g, 79.47 mmol) in dimethylformamide (50 mL) was added dropwise and the reaction heated at 60° C. for 16 h. The reaction was quenched with water (1000 mL) at 0° C. and extracted with ethyl acetate (2×200 mL). The combined organic layers were dried over anhydrous Na₂SO₄, filtered, and concentrated in vacuo. The crude material was then purified by combi-flash column chromatography, by eluting with 30% ethyl acetate in heptane, to afford benzyl 4-((1-(tert-butoxycarbonyl) piperidin-4-yl) methoxy) piperidine-1-carboxylate (Intermediate 113, 6.5 g, 21%) as a colourless oil. ¹H NMR (400 MHz, CDCl₃) δ ppm 1.07-1.19 (m, 2H), 1.45 (s, 9H), 1.69-1.80 (m, 5H), 2.64-2.72 (m, 2H), 3.22-3.28 (m, 4H), 3.41-3.45 (m, 2H), 3.70-3.80 (m, 2H), 4.08-4.12 (m, 2H), 4.67-4.70 (m, 1H), 5.12 (s, 2H), 7.28-7.40 (m, 5H).

### Step 2

### Intermediate 114: tert-butyl 4-((piperidin-4-yloxy)methyl)piperidine-1-carboxylate

To a solution of benzyl 4-((1-(tert-butoxycarbonyl)piperidin-4-yl)methoxy)piperidine-1-carboxylate (Intermediate 113, 6.50 g, 15.0 mmol) in methanol (300 mL) was added 10% Pd/C (1.75 g) under a nitrogen atmosphere. The suspension was de-gassed under vacuum and purged with H₂ three times. The mixture was then stirred at room temperature for 16 h under a H₂ (35 psi). The reaction mixture was filtered through celite and washed with MeOH (2×50 mL). The filtrate was concentrated in vacuo to afford tert-butyl 4-((piperidin-4-yloxy)methyl) piperidine-1-carboxylate (Intermediate 114, 4.5 g) as a colourless oil, which was used for the next step without further purification. ¹H NMR (400 MHz, CDCl₃) δ ppm 1.00-1.19 (m, 2H), 1.45 (s, 9H), 1.68-1.74 (m, 5H), 1.89-2.04 (m, 2H), 2.66-2.74 (m, 2H), 2.82-2.93 (m, 2H), 3.09-3.19 (m, 2H), 3.22-3.31 (m, 2H), 3.42-3.48 (m, 1H), 4.04-4.12 (m, 2H), 4.70 (br s, 1H).

### Step 3

### Intermediate 115: tert-butyl 4-(((1-(5-carbamoylpyridin-2-yl)piperidin-4-yl)oxy)methyl)piperidine-1-carboxylate

To a solution of tert-butyl 4-((piperidin-4-yloxy)methyl)piperidine-1-carboxylate (Intermediate 114, 1.6 g, 5.4 mmol) and 6-chloronicotinamide (CAS No: 6271-78-9, 0.76 g, 4.8 mmol) in dimethylformamide (10 mL) was added potassium carbonate (2.2 g, 16.0 mmol) and the reaction mixture was heated at 130° C. for 16 h. The reaction mixture was poured in ice cold water (100 mL), a precipitate formed which was filtered, washed with water (50 mL) and dried in vacuo to afford tert-butyl 4-(((1-(5-carbamoylpyridin-2-yl)piperidin-4-yl)oxy)methyl) piperidine-1-carboxylate (Intermediate 115, 1.30 g, 58%) as an off white solid. ¹H NMR (400 MHz, DMSO-d₆) δ ppm 0.96-1.08 (m, 2H), 1.38 (s, 9H), 1.42-1.44 (m, 2H), 1.61-1.68 (m, 3H), 1.81-1.86 (m, 2H), 2.61-2.70 (m, 2H), 3.28-3.32 (m, 4H), 3.48-3.54 (m, 1H), 3.90-3.98 (m, 4H), 6.84 (d, J=8.77 Hz, 1H), 7.10 (br s, 1H), 7.73 (br s, 1H), 7.93 (d, J=8.77 Hz, 1H), 8.59 (s, 1H). Mass spec: m/z 419.3 [M+H]⁺.

### Step 4

### Intermediate 116: tert-butyl (R)-6-(6-(4-((1-(tert-butoxycarbonyl)piperidin-4-yl)methoxy)piperidin-1-yl) nicotinamido)-2-(1-methylpyrrolidin-2-yl)-1H-pyrrolo[3,2-c]pyridine-1-carboxylate

To a mixture of tert-butyl 4-(((1-(5-carbamoylpyridin-2-yl)piperidin-4-yl)oxy)methyl)piperidine-1-carboxylate (Intermediate 115, 0.80 g, 1.91 mmol) and tert-butyl (R)-6-bromo-2-(1-methylpyrrolidin-2-yl)-1H-pyrrolo[3,2-c]pyridine-1-carboxylate (Intermediate 4, 0.79 g, 2.10 mmol) in 1,4-dioxane (16 mL) was added cesium carbonate (1.88 g, 5.73 mmol) at room temperature. The reaction mixture was purged with argon for 15 min followed by the addition of Pd₂(dba)₃ (0.28 g, 0.27 mmol) and Xantphos (0.34 g, 0.57 mmol). The reaction mixture was further purged with argon for 10 min and heated at 100° C. for 2 h. The reaction mixture was concentrated in vacuo and the crude material was purified by combi-flash column chromatography, by eluting with 3% MeOH in DCM, to afford tert-butyl (R)-6-(6-(4-((1-(tert-butoxycarbonyl)piperidin-4-yl)methoxy)piperidin-1-yl) nicotinamido)-2-(1-methylpyrrolidin-2-yl)-1H-pyrrolo[3,2-c]pyridine-1-carboxylate (Intermediate 116, 1.1 g, 80%) as a brown solid. ¹H NMR (400 MHz, DMSO-d₆) δ ppm 0.90-1.08 (m, 4H), 1.35 (s, 9H), 1.38-1.40 (m, 2H), 1.49-1.61 (m, 3H), 1.65 (s, 9H), 1.68-1.77 (m, 3H), 1.81-1.90 (m, 2H), 2.24-2.29 (m, 1H), 2.32 (s, 3H), 2.62-2.66 (m, 3H), 3.04-3.10 (m, 2H), 3.48-3.52 (m, 2H), 3.79-4.08 (m, 5H), 6.70 (s, 1H), 6.85 (d, J=8.71 Hz, 1H), 8.08-8.12 (m, 1H), 8.53 (s, 1H), 8.75 (s, 1H), 8.87 (s, 1H), 10.45 (br s, 1H). Mass spec: m/z 718.1 [M+H]⁺.

### Step 5

### Intermediate 117: (R)—N-(2-(1-methylpyrrolidin-2-yl)-1H-pyrrolo[3,2-c]pyridin-6-yl)-6-(4-(piperidin-4-ylmethoxy) piperidin-1-yl)nicotinamide dihydrochloride

To a solution of tert-butyl (R)-6-(6-(4-((1-(tert-butoxycarbonyl) piperidin-4-yl)methoxy)piperidin-1-yl) nicotinamido)-2-(1-methylpyrrolidin-2-yl)-1H-pyrrolo[3,2-c]pyridine-1-carboxylate (Intermediate 116, 1.00 g, 1.39 mmol) in 1,4-dioxane (20 mL) was added 4M HCl in 1,4-dioxane (20 mL) at 0° C. and the reaction mixture was stirred at room temperature for 16 h. The reaction mixture was concentrated in vacuo and washed with diethyl ether (20 mL) to afford (R)—N-(2-(1-methylpyrrolidin-2-yl)-1H-pyrrolo[3,2-c]pyridin-6-yl)-6-(4-(piperidin-4-ylmethoxy)piperidin-1-yl)nicotinamide dihydrochloride (Intermediate 117, 0.90 g) as an off white solid, which was used for the next step without further purification. Mass spec: m/z 518.5 [M+H]⁺.

### Step 6

### Example 18: 6-(4-((1-(2-(2,6-dioxopiperidin-3-yl)-1,3-dioxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)nicotinamide

To a solution of (R)—N-(2-(1-methylpyrrolidin-2-yl)-1H-pyrrolo[3,2-c]pyridin-6-yl)-6-(4-(piperidin-4-ylmethoxy) piperidin-1-yl)nicotinamide dihydrochloride (Intermediate 117, 0.50 g, 0.90 mmol) in dimethyl sulfoxide (10 mL) was added N,N-diisopropylethylamine (0.78 mL, 4.51 mmol) followed by 2-(2,6-dioxopiperidin-3-yl)-4-fluoroisoindoline-1,3-dione (CAS No: 835616-60-9, 0.24 g, 0.90 mmol) and the reaction mixture was heated at 120° C. for 2 h. The reaction mixture was quenched with ice cold water (50 mL), the precipitate formed was filtered and dried in vacuo. The crude precipitate was purified by preparative HPLC (Method A) to afford 6-(4-((1-(2-(2,6-dioxopiperidin-3-yl)-1,3-dioxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)nicotinamide (Example 18, 0.16 g, 24%) as a yellow solid. ¹H NMR (400 MHz, DMSO-d₆) δ ppm 1.37-1.53 (m, 4H), 1.69-1.95 (m, 9H), 1.98-2.06 (m, 1H), 2.09-2.14 (m, 1H), 2.16 (s, 3H), 2.22-2.30 (m, 1H), 2.56-2.62 (m, 2H), 2.82-2.95 (m, 3H), 3.10-3.19 (m, 1H), 3.36-3.41 (m, 4H), 3.53-3.63 (m, 1H), 3.66-3.74 (m, 2H), 3.98-4.04 (m, 2H), 5.06-5.11 (m, 1H), 6.37 (s, 1H), 6.89 (d, J=9.20 Hz, 1H), 7.30-7.36 (m, 2H), 7.66-7.70 (m, 1H), 8.12-8.14 (m, 1H), 8.16 (s, 1H), 8.48 (s, 1H), 8.79 (s, 1H), 10.24 (s, 1H), 11.07 (s, 1H), 11.32 (s, 1H). Mass spec: m/z: 774.0 [M+H]⁺.

### Example 19 was Synthesised Following Scheme 22

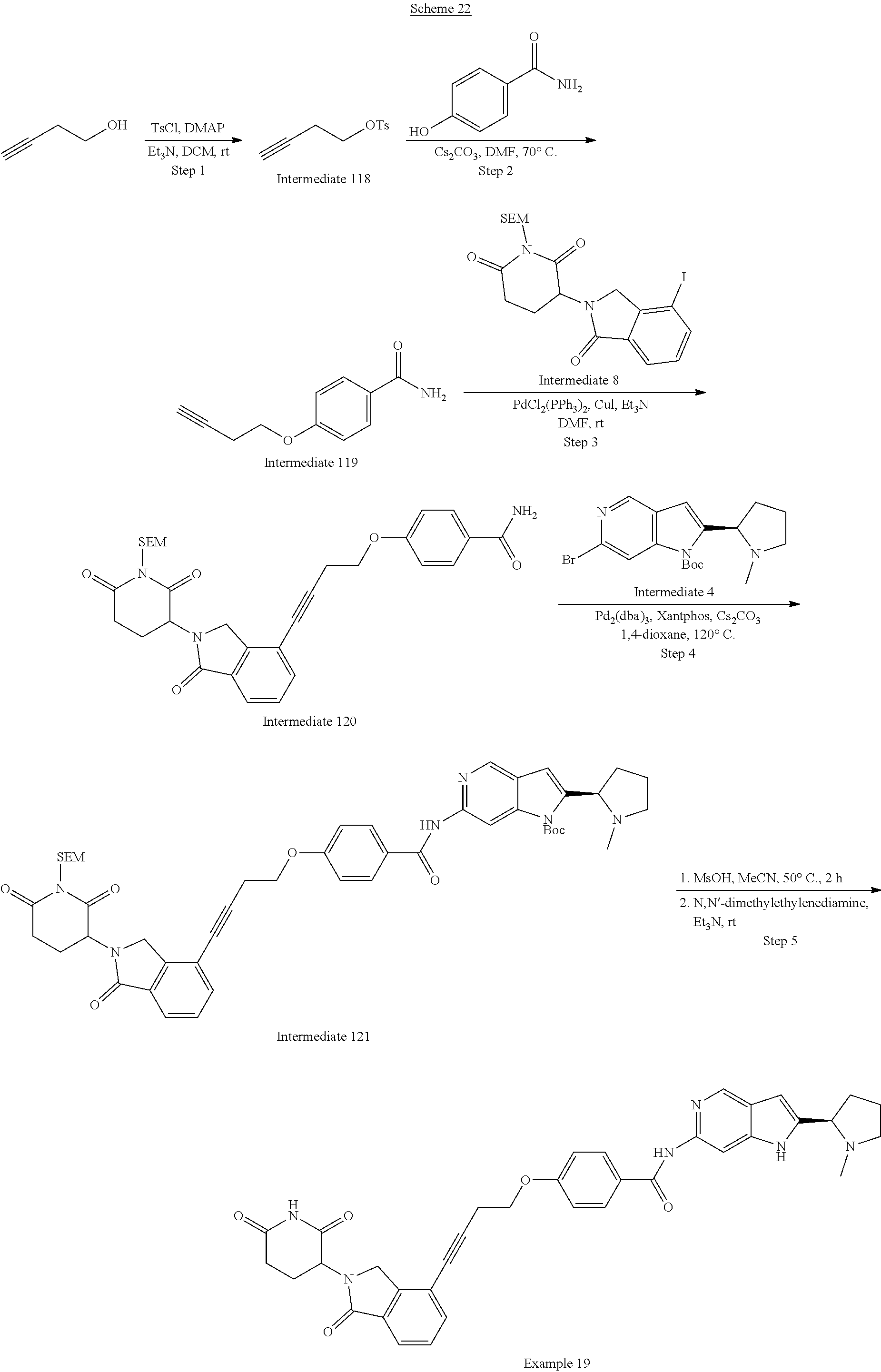

### Step 1

### Intermediate 118: but-3-yn-1-yl 4-methylbenzenesulfonate

To a solution of but-3-yn-1-ol (CAS No: 927-74-2, 10.0 g, 142.7 mmol) in dichloromethane (240 mL) was added triethylamine (21.87 g, 214.0 mmol) and 4-dimethylaminopyridine (1.76 g, 14.27 mmol) and the reaction was stirred at 0° C. for 15 min. p-Toluenesulfonyl chloride (33.3 g, 171.2 mmol) was added and the reaction was stirred for 16 h at room temperature. The reaction mixture was diluted with 1N HCl (150 mL) and extracted with dichloromethane (2×200 mL). The combined organic layers were washed with NaHCO₃ solution (2×100 mL) and brine solution (2×100 mL), dried over anhydrous Na₂SO₄ and concentrated in vacuo to afford but-3-yn-1-yl 4-methylbenzenesulfonate (Intermediate 118, 25.0 g, 78%) as yellow colour oil, which was used for the next step without further purification. ¹H NMR (400 MHz, DMSO-d₆) δ ppm 2.43 (t, J=2.60 Hz, 1H), 2.46 (s, 3H), 2.87 (t, J=2.60 Hz, 1H), 3.31 (t, J=7.0 Hz, 1H), 4.04 (t, J=7.0 Hz, 2H), 7.50 (d, J=8.0 Hz, 2H), 7.80 (d, J=8.0 Hz, 2H).

### Step 2

### Intermediate 119: 4-(but-3-yn-1-yloxy)benzamide

To a solution of but-3-yn-1-yl 4-methylbenzenesulfonate (Intermediate 118, 8.18 g, 36.5 mmol) in dimethylformamide (150 mL) was added 4-hydroxybenzamide (CAS No: 619-57-8, 2.00 g, 14.6 mmol) followed by cesium carbonate (14.3 g, 43.8 mmol) at room temperature. The reaction mixture was then heated at 70° C. for 16 h. Water (100 mL) was added and the aqueous phase was extracted with ethyl acetate (2×60 mL). The combined organic phases were washed with brine solution (2×60 mL), dried over anhydrous Na₂SO₄, filtered, and concentrated in vacuo. The crude material was purified by combi-flash chromatography, by eluting with 50% ethyl acetate in heptane, to afford 4-(but-3-yn-1-yloxy)benzamide (Intermediate 119, 0.38 g, 14%) as a white solid. ¹H NMR (400 MHz, DMSO-d₆) δ ppm 2.65 (t, J=7.20 Hz, 2H) 2.89 (t, J=7.20 Hz, 1H) 4.11 (t, J=7.20 Hz, 2H) 6.97-6.99 (m, 2H) 7.17 (br s, 1H) 7.82-7.84 (m, 3H).

### Step 3

### Intermediate 120: 4-((4-(2-(2,6-dioxo-1-((2-(trimethylsilyl)ethoxy)methyl)piperidin-3-yl)-1-oxoisoindolin-4-yl)but-3-yn-1-yl)oxy)benzamide

To a solution of 3-(4-iodo-1-oxoisoindolin-2-yl)-1-((2-(trimethylsilyl)ethoxy)methyl)piperidine-2,6-dione (Intermediate 119, 0.97 g, 1.94 mmol) in dimethylformamide (3 mL) was added 4-(but-3-yn-1-yloxy)benzamide (Intermediate 8, 0.35 g, 1.84 mmol) followed by triethylamine (9.07 mL, 64.74 mmol). The reaction mixture was purged with argon gas for 15 min then PdCl₂(PPh₃)₂ (0.13 g, 0.18 mmol) and copper(I) iodide (0.03 g, 0.18 mmol) were added. The reaction mixture was further purged with argon gas for 10 min and stirred at room temperature for 3 h. The reaction mixture was diluted with water (50 mL) and extracted with ethyl acetate (3×50 mL). The combined organic layers were washed with brine solution (2×30 mL), dried over anhydrous Na₂SO₄, filtered, and concentrated in vacuo. The crude material was purified by combi-flash chromatography, by eluting with ethyl acetate, to afford 4-((4-(2-(2,6-dioxo-1-((2-(trimethylsilyl)ethoxy)methyl)piperidin-3-yl)-1-oxoisoindolin-4-yl) but-3-yn-1-yl)oxy)benzamide (Intermediate 120, 0.35 g, 34%) as a white solid. ¹H NMR (400 MHz, DMSO-d₆) δ ppm −0.03 (s, 9H), 0.76-0.92 (m, 2H), 1.97-2.12 (m, 1H), 2.27-2.41 (m, 1H), 2.66-2.85 (m, 2H), 2.93-3.14 (m, 3H), 3.46-3.59 (m, 2H), 4.20-4.32 (m, 2H), 4.41-4.46 (m, 1H), 4.99-5.12 (m, 2H), 5.23-5.27 (m, 1H), 7.02 (d, J=8.40 Hz, 2H), 7.17 (br s, 1H), 7.54 (t, J=7.20 Hz, 1H), 7.66 (d, J=7.20 Hz, 1H), 7.74 (d, J=7.21 Hz, 1H), 7.82-7.86 (m, 3H). Mass spec: m/z: 560.0 [M−H]⁻.

### Step 4

### Intermediate 121: tert-butyl 6-(4-((4-(2-(2,6-dioxo-1-((2-(trimethylsilyl)ethoxy)methyl)piperidin-3-yl)-1-oxoisoindolin-4-yl)but-3-yn-1-yl)oxy)benzamido)-2-((R)-1-methylpyrrolidin-2-yl)-1H-pyrrolo[3,2-c]pyridine-1-carboxylate

To a solution of 4-((4-(2-(2,6-dioxo-1-((2-(trimethylsilyl)ethoxy)methyl)piperidin-3-yl)-1-oxoisoindolin-4-yl) but-3-yn-1-yl)oxy)benzamide (Intermediate 120, 0.35 g, 0.62 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 0.28 g, 0.74 mmol) followed by cesium carbonate (0.61 g, 1.86 mmol) at room temperature. The reaction mixture was purged with argon for 15 min followed by Pd₂(dba)₃ (0.11 g, 0.12 mmol) and Xantphos (0.14 g, 0.24 mmol). The reaction mixture was purged with argon for 10 min and heated at 120° C. for 90 min. The reaction mixture was diluted with water (50 mL) and extracted with ethyl acetate (2×100 mL). The organic layers were separated, dried over anhydrous Na₂SO₄, and concentrated in vacuo. The crude material was purified by combi-flash chromatography, by eluting with 2% MeOH in DCM, to afford tert-butyl 6-(4-((4-(2-(2,6-dioxo-1-((2-(trimethylsilyl)ethoxy)methyl)piperidin-3-yl)-1-oxoisoindolin-4-yl)but-3-yn-1-yl)oxy)benzamido)-2-((R)-1-methylpyrrolidin-2-yl)-1H-pyrrolo[3,2-c]pyridine-1-carboxylate (Intermediate 121, 0.31 g, 58%) as a brown solid. ¹H NMR (400 MHz, DMSO-d₆) δ ppm 0.00 (s, 9H), 0.77-0.90 (m, 2H), 1.35-1.40 (m, 1H), 1.69 (s, 9H), 1.79-1.98 (m, 4H), 2.35 (s, 3H), 2.37-2.39 (m, 3H), 2.75-2.79 (m, 1H), 3.00-3.13 (m, 3H), 3.50-3.52 (m, 2H), 3.88-3.94 (m, 1H), 4.26-4.38 (m, 3H), 4.48-4.53 (m, 1H), 5.00-5.06 (m, 2H), 5.25-4.27 (m, 1H), 6.75 (s, 1H), 7.04-7.12 (m, 2H), 7.25-7.40 (m, 2H), 7.62-7.66 (m, 1H), 8.02-8.13 (m, 2H), 8.60 (s, 1H), 8.92 (s, 1H), 10.78 (s, 1H).

### Step 5

### Example 19: 4-((4-(2-(2,6-dioxopiperidin-3-yl)-1-oxoisoindolin-4-yl)but-3-yn-1-yl)oxy)-N-(2-((R)-1-methylpyrrolidin-2-yl)-1H-pyrrolo[3,2-c]pyridin-6-yl)benzamide

To a solution of tert-butyl 6-(4-((4-(2-(2,6-dioxo-1-((2-(trimethylsilyl)ethoxy)methyl)piperidin-3-yl)-1-oxoisoindolin-4-yl)but-3-yn-1-yl)oxy)benzamido)-2-((R)-1-methylpyrrolidin-2-yl)-1H-pyrrolo[3,2-c]pyridine-1-carboxylate (Intermediate 121, 0.30 g, 0.34 mmol) in acetonitrile (10 mL) was added methanesulfonic acid (0.34 mL, 5.22 mmol) at room temperature and the reaction mixture heated at 50° C. for 2 h. After cooling to room temperature, N,N′-dimethylethylenediamine (0.25 mL, 2.09 mmol) followed by triethylamine (0.73 mL, 5.22 mmol) were added and the reaction stirred at room temperature for 2 h. The reaction mixture was then concentrated under reduced pressure, washed with ether (2×50 mL) and n-pentane (2×40 mL) and then dried to obtain crude compound. The crude material was purified by preparative HPLC (Method A) to afford 4-((4-(2-(2,6-dioxopiperidin-3-yl)-1-oxoisoindolin-4-yl)but-3-yn-1-yl)oxy)-N-(2-((R)-1-methylpyrrolidin-2-yl)-1H-pyrrolo[3,2-c]pyridin-6-yl)benzamide (Example 19, 0.01 g, 8%) as an off white solid. ¹H NMR (400 MHz, DMSO-d₆) δ ppm 1.75-1.94 (m, 3H), 1.96-2.04 (m, 1H), 2.10-2.14 (m, 1H), 2.17 (s, 3H), 2.22-2.30 (m, 1H), 2.31-2.37 (m, 1H), 2.55-2.64 (m, 2H), 2.86-2.96 (m, 1H), 3.01-3.55 (m, 2H), 3.11-3.19 (m, 1H), 4.26-4.35 (m, 3H), 4.39-4.48 (m, 1H), 5.11-5.16 (m, 1H), 6.38 (s, 1H), 7.06-7.11 (m, 2H), 7.50-7.57 (m, 1H), 7.66-7.71 (m, 1H), 7.73-7.78 (m, 1H), 8.03-8.07 (m, 2H), 8.17 (s, 1H), 8.49 (s, 1H), 10.28 (br s, 1H), 11.02 (br s, 1H), 11.34 (br s, 1H). Mass spec: m/z: 631.3 [M+H]⁺.

### Example 20 was Synthesised Following Scheme 23

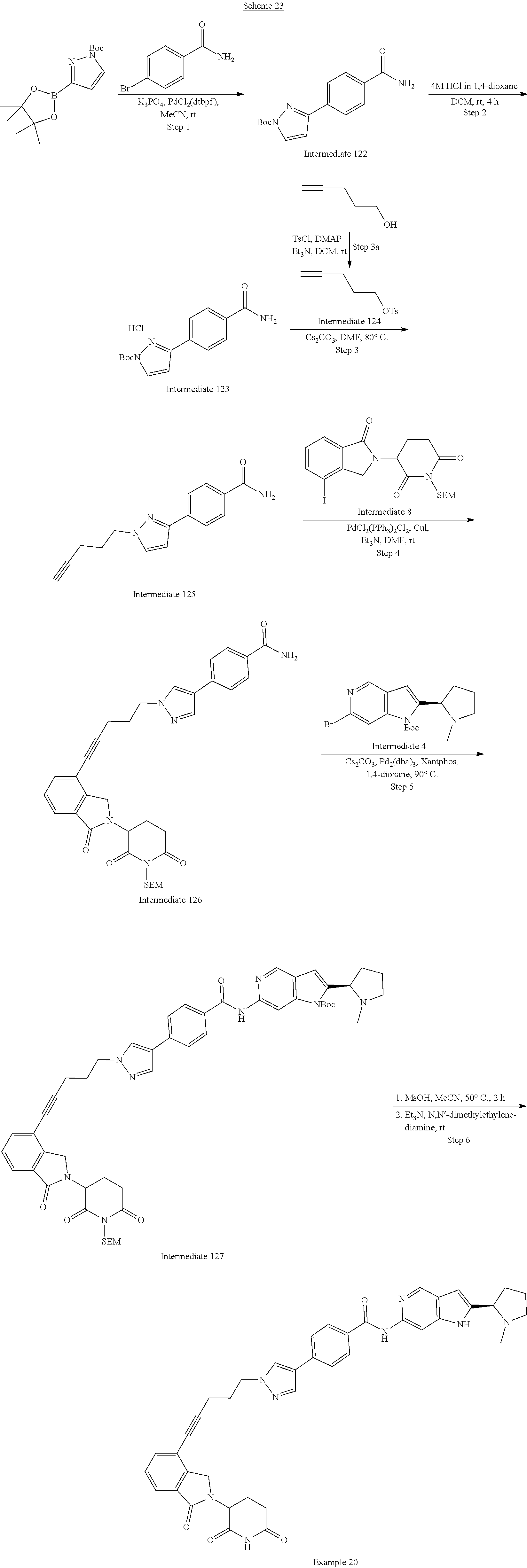

### Step 1

### Intermediate 122: tert-butyl 3-(4-carbamoylphenyl)-1H-pyrazole-1-carboxylate

A solution of 4-bromobenzamide (CAS No: 698-67-9, 3.0 g, 15 mmol), tert-butyl 3-(4,4,5,5-tetramethyl-1,3,2-dioxaborolan-2-yl)-1H-pyrazole-1-carboxylate (CAS No: 914672-66-5, 4.9 g, 16 mmol) and K₃PO₄ (9.8 g, 45 mmol) in acetonitrile (60 mL) at room temperature was purged with argon gas for 5 min. PdCl₂(dtbpf) (0.80 g, 1.2 mmol) was added at room temperature and stirred for 4 h. The reaction mixture was poured into water (100 mL) and extracted with ethyl acetate (2×100 mL). The combined organic layers were washed with saturated Na₂S₂O₃ (150 mL), dried over anhydrous Na₂SO₄, filtered and concentrated in vacuo to obtain crude compound. The crude material was purified by combi-flash column chromatography, by eluting with 80% ethyl acetate in heptane, to afford tert-butyl 3-(4-carbamoylphenyl)-1H-pyrazole-1-carboxylate (Intermediate 122, 2.2 g, 51%) as a yellow gum. ¹H NMR (400 MHz, DMSO-d₆) δ ppm 1.62 (s, 9H), 7.14 (d, J=2.07 Hz, 1H), 7.40 (br s, 1H), 7.94-8.05 (m, 4H), 8.03 (s, 1H), 8.36 (d, J=2.07 Hz, 1H). Mass spec: m/z: 288.06 [M+H]⁺.

### Step 2

### Intermediate 123: 4-(1H-pyrazol-3-yl)benzamide hydrochloride

To a solution of tert-butyl 3-(4-carbamoylphenyl)-1H-pyrazole-1-carboxylate (Intermediate 122, 2.20 g, 7.7 mmol) in dichloromethane (20 mL) was added 4M hydrochloric acid in 1,4-dioxane (19 mL, 77 mmol) at 0° C. The reaction mixture was then stirred at room temperature for 4 h. The reaction mixture was concentrated in vacuo and the resulting solid was washed with diethyl ether (20 mL) and dried under reduced pressure to afford 4-(1H-pyrazol-3-yl)benzamide hydrochloride (Intermediate 123, 1.50 g) as an off white solid, which was used in the next step without further purification. ¹H NMR (400 MHz, DMSO-d₆) δ ppm 6.81 (s, 1H), 7.33 (br s, 1H), 7.76 (s, 1H), 7.86-7.92 (m, 4H), 7.95 (br s, 1H). Mass spec: m/z: 188.02 [M+H]⁺.

### Step 3a

### Intermediate 124: pent-4-yn-1-yl 4-methylbenzenesulfonate

To solution of pent-4-yn-1-ol (CAS No: 5390-04-5, 5.0 g, 59 mmol) in dichloromethane (100 mL) was added triethylamine (19 g, 180 mmol) at 0° C. p-toluenesulfonylchloride (18 g, 89 mmol) followed by 4-dimethylaminopyridine (0.76 g, 5.9 mmol) were added and the reaction mixture was stirred at room temperature for 16 h. The reaction mixture was poured into ice cold water (100 mL) and extracted with ethyl acetate (2×150 mL). The combined organic phases were dried over anhydrous Na₂SO₄, filtered and concentrated in vacuo to afford pent-4-yn-1-yl 4-methylbenzenesulfonate (Intermediate 124, 7.00 g, 49%) as a gum. ¹H NMR (400 MHz, CDCl₃) δ ppm 1.83-1.89 (m, 3H), 2.24-2.28 (m, 2H), 2.45 (s, 3H), 4.15 (t, J=6.14 Hz, 2H), 7.34 (d, J=7.89 Hz, 2H), 7.80 (d, J=8.33 Hz, 2H). Mass spec: m/z: 239.0 [M+H]⁺.

### Step 3

### Intermediate 125: 4-(1-(pent-4-yn-1-yl)-1H-pyrazol-3-yl)benzamide

A solution of pent-4-ynyl 4-methylbenzenesulfonate (Intermediate 124, 2.05 g, 8.58 mmol), 4-(1H-pyrazol-3-yl)benzamide hydrochloride (Intermediate 123, 1.28 g, 5.72 mmol) and cesium carbonate (5.89 g, 17.2 mmol) in 1,4-dioxane (25 mL) was heated at 80° C. for 3 h. The reaction mixture was poured into water (100 mL) and extracted with ethyl acetate (2×150 mL). The combined organic phases were dried over anhydrous Na₂SO₄, filtered, and concentrated in vacuo to obtain crude compound. The crude material was purified by combi-flash column chromatography, by eluting with 80% ethyl acetate in heptane, to afford 4-(1-(pent-4-yn-1-yl)-1H-pyrazol-3-yl)benzamide (Intermediate 125, 1.25 g, 86%) as an off white solid. ¹H NMR (400 MHz, DMSO-d₆) δ ppm 1.98-2.02 (m, 2H), 2.16-2.18 (m, 2H), 2.86 (br s, 1H), 4.21-4.24 (m, 2H), 6.79 (s, 1H), 7.31 (br s, 1H), 7.76-7.91 (m, 5H), 7.96 (br s, 1H). Mass spec: m/z: 254.09 [M+H]⁺.

### Step 4

### Intermediate 126: 4-(1-(5-(2-(2,6-dioxo-1-((2-(trimethylsilyl)ethoxy)methyl)piperidin-3-yl)-1-oxoisoindolin-4-yl) pent-4-yn-1-yl)-1H-pyrazol-3-yl)benzamide

A solution of 3-(4-iodo-1-oxo-isoindolin-2-yl)-1-(2-trimethylsilylethoxymethyl)piperidine-2,6-dione (Intermediate 8, 1.0 g, 2.0 mmol), 4-(1-pent-4-ynylpyrazol-3-yl)benzamide (Intermediate 125, 0.51 g, 2.0 mmol) and Et₃N (8.3 g, 78 mmol) in dimethylformamide (1 mL) was purged with argon gas for 15 min. Copper(I) iodide (0.040 g, 0.20 mmol) and PdCl₂(PPh₃)₂ (0.15 g, 0.20 mmol) were added and the reaction was stirred at room temperature and stirred for 1 h. The reaction mixture was poured into water (80 mL) and extracted with ethyl acetate (2×80 mL). The combined organic phases were dried over anhydrous Na₂SO₄, filtered, and concentrated in vacuo to obtain crude compound. The crude material was purified by combi-flash column chromatography, by eluting with 3% MeOH in DCM, to afford 4-(1-(5-(2-(2,6-dioxo-1-((2-(trimethylsilyl) ethoxy)methyl)piperidin-3-yl)-1-oxoisoindolin-4-yl) pent-4-yn-1-yl)-1H-pyrazol-3-yl)benzamide (Intermediate 126, 1.0 g, 80%) as brown solid. ¹H NMR (400 MHz, DMSO-d₆) δ ppm −0.05 (s, 9H), 0.79-0.84 (m, 2H), 1.99-2.06 (m, 2H), 2.10-2.17 (m, 2H), 2.37-2.40 (m, 2H), 2.73-2.79 (m, 1H), 3.01-3.10 (m, 1H), 3.46-3.54 (m, 2H), 4.27-4.31 (m, 3H), 4.48-4.52 (m, 1H), 4.99-5.08 (m, 2H), 5.23-5.28 (m, 1H), 6.79 (s, 1H), 7.31 (s, 1H), 7.51-7.55 (m, 1H), 7.67 (d, J=7.46 Hz, 1H), 7.72 (d, J=7.88 Hz, 1H), 7.81-7.85 (m, 3H), 7.87-7.90 (m, 2H), 7.93-7.95 (m, 1H). Mass spec: m/z: 626.33 [M+H]⁺.

### Step 5

### Intermediate 127: tert-butyl 6-(4-(1-(5-(2-(2,6-dioxo-1-((2-(trimethylsilyl)ethoxy)methyl)piperidin-3-yl)-1-oxoisoindolin-4-yl)pent-4-yn-1-yl)-1H-pyrazol-3-yl)benzamido)-2-((R)-1-methylpyrrolidin-2-yl)-1H-pyrrolo[3,2-c]pyridine-1-carboxylate

A solution of 4-(1-(5-(2-(2,6-dioxo-1-((2-(trimethylsilyl)ethoxy)methyl)piperidin-3-yl)-1-oxoisoindolin-4-yl)pent-4-yn-1-yl)-1H-pyrazol-3-yl)benzamide (Intermediate 126, 1.05 g, 1.683 mmol), tert-butyl (R)-6-bromo-2-(1-methylpyrrolidin-2-yl)-1H-pyrrolo[3,2-c]pyridine-1-carboxylate (Intermediate 4, 0.8 g, 2.104 mmol) and Cs₂CO₃ (1.80 g, 5.26 mmol) in 1,4-dioxane (10 mL) at room temperature was purged with argon gas for 15 min. To the reaction mixture was added Pd₂(dba)₃ (0.27 g, 0.32 mmol) followed by Xantphos (0.38 g, 0.63 mmol) at room temperature and the reaction mixture was heated at 90° C. for 1 h. The reaction mixture was filtered through celite, washed with DCM (20 mL) and concentrated in vacuo to obtain crude compound. The crude material was purified by combi-flash column chromatography, by eluting with 10% MeOH in DCM, to afford tert-butyl 6-(4-(1-(5-(2-(2,6-dioxo-1-((2-(trimethylsilyl)ethoxy)methyl)piperidin-3-yl)-1-oxoisoindolin-4-yl)pent-4-yn-1-yl)-1H-pyrazol-3-yl)benzamido)-2-((R)-1-methylpyrrolidin-2-yl)-1H-pyrrolo[3,2-c]pyridine-1-carboxylate (Intermediate 127, 0.40 g, 21%) as brown solid. ¹H NMR (400 MHz, DMSO-d₆) δ ppm −0.05 (s, 9H), 0.79-0.81 (m, 2H), 1.70 (s, 9H), 1.75-1.89 (m, 3H), 2.05-2.17 (m, 4H), 2.36 (s, 3H), 2.37-2.39 (m, 3H), 2.75-2.79 (m, 1H), 3.00-3.13 (m, 3H), 3.50-3.52 (m, 2H), 3.90-3.92 (m, 1H), 4.28-4.32 (m, 3H), 4.48-4.53 (m, 1H), 5.00-5.06 (m, 2H), 5.25-4.27 (m, 1H), 6.76 (s, 1H), 6.84 (s, 1H), 7.53-7.55 (m, 1H), 7.67-7.74 (m, 2H), 7.87-7.92 (m, 3H), 8.08 (d, J=7.20 Hz, 2H), 8.60 (s, 1H), 8.95 (s, 1H), 10.68 (br s, 1H). Mass spec: m/z: 925.2 [M+H]⁺.

### Step 6

### Example 20: 4-(1-(5-(2-(2,6-dioxopiperidin-3-yl)-1-oxoisoindolin-4-yl)pent-4-yn-1-yl)-1H-pyrazol-3-yl)-N-(2-((R)-1-methylpyrrolidin-2-yl)-1H-pyrrolo[3,2-c]pyridin-6-yl)benzamide

To a solution of tert-butyl 6-(4-(1-(5-(2-(2,6-dioxo-1-((2-(trimethylsilyl)ethoxy)methyl)piperidin-3-yl)-1-oxoisoindolin-4-yl)pent-4-yn-1-yl)-1H-pyrazol-3-yl)benzamido)-2-((R)-1-methylpyrrolidin-2-yl)-1H-pyrrolo[3,2-c]pyridine-1-carboxylate (Intermediate 127, 0.40 g, 0.43 mmol) in acetonitrile (10 mL) was added methanesulfonic acid (0.44 g, 4.32 mmol) at 0° C. and the reaction mixture was heated to 50° C. for 2 h. N,N′-dimethylethylenediamine (0.20 g, 2.16 mmol) followed by triethylamine (0.92 g, 8.65 mmol) were added and the reaction stirred at room temperature and stirred for 16 h. The reaction mixture was concentrated in vacuo and quenched with cold water (60 mL) to obtain solid precipitate, which was filtered and dried. The crude material was purified by preparative HPLC (Method A) to afford 4-(1-(5-(2-(2,6-dioxopiperidin-3-yl)-1-oxoisoindolin-4-yl)pent-4-yn-1-yl)-1H-pyrazol-3-yl)-N-(2-((R)-1-methylpyrrolidin-2-yl)-1H-pyrrolo[3,2-c]pyridin-6-yl)benzamide (Example 20, 0.12 g, 39%) as an off white solid. ¹H NMR (400 MHz, DMSO-d₆) δ ppm 1.75-1.95 (m, 3H), 1.99-2.03 (m, 1H), 2.11-2.16 (m, 3H), 2.17 (s, 3H), 2.23-2.30 (m, 1H), 2.40-2.44 (m, 2H), 2.52-2.57 (m, 3H), 2.86-2.95 (m, 1H), 3.12-3.16 (m, 1H), 4.31-4.37 (m, 3H), 4.47-4.51 (m, 1H), 5.11-5.15 (m, 1H), 6.40 (s, 1H), 6.84 (d, J=2.25 Hz, 1H), 7.52 (t, J=7.63 Hz, 1H), 7.67 (d, J=6.88 Hz, 1H), 7.71 (d, J=7.63 Hz, 1H), 7.87 (d, J=2.40 Hz, 1H), 7.91 (d, J=8.38 Hz, 2H), 8.09 (d, J=8.40 Hz, 2H), 8.21 (s, 1H), 8.51 (s, 1H), 10.43 (s, 1H), 10.99 (s, 1H), 11.37 (s, 1H). Mass spec: m/z: 695.1 [M+H]⁺.

### Example 21 was Synthesised Following Scheme 24

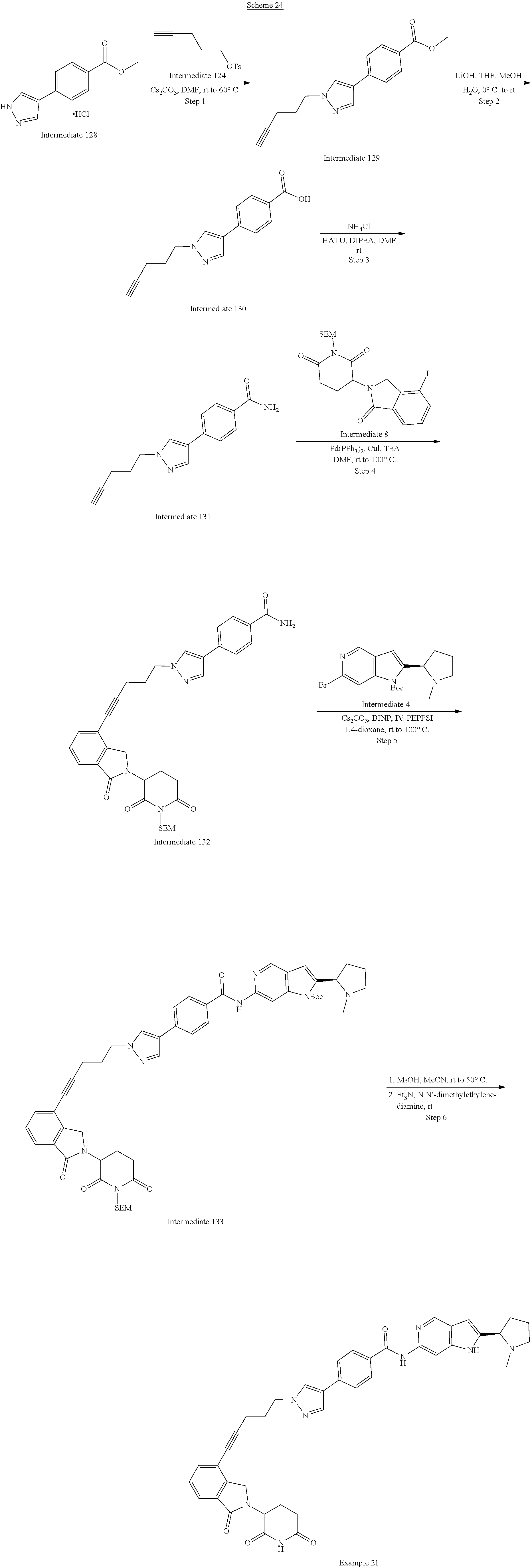

### Step 1

### Intermediate 129: methyl 4-(1-(pent-4-yn-1-yl)-1H-pyrazol-4-yl)benzoate

To a solution of pent-4-yn-1-yl 4-methylbenzenesulfonate (Intermediate 124, 7 g, 85.22 mmol) in DMF (70 mL) was added cesium carbonate (83.4 g, 255.7 mmol) and methyl 4-(1H-pyrazol-4-yl)benzoate hydrochloride (Intermediate 128, 4.3 g, 85.22 mmol) at room temperature. The reaction mixture was heated to 60° C. for 16 h. Progress of the reaction was monitored by TLC and LCMS. After completion, the reaction mixture was cooled to room temperature, treated with water (100 mL) and extracted with EtOAc (2×200 mL). The organic layer was dried over Na₂SO₄ and concentrated in vacuo. The obtained residue was purified by normal phase MPLC (25-30% in EtOAc in heptane) to afford methyl 4-(1-(pent-4-yn-1-yl)-1H-pyrazol-4-yl)benzoate (Intermediate 129, 4.3 g, 19%) as an off-white solid. ¹H NMR (400 MHz, DMSO-d₆) δ ppm 1.99-2.00 (m, 2H), 2.17-2.19 (m, 2H), 2.86 (s, 1H), 3.85 (s, 3H), 4.21-4.32 (m, 2H), 7.73 (d, J=6.85 Hz, 2H), 7.93 (d, J=6.85 Hz, 2H) 8.00 (s, 1H), 8.34 (s, 1H). Mass spec: m/z 269.1 [M+H]⁺.

### Step 2

### Intermediate 130: 4-(1-(pent-4-yn-1-yl)-1H-pyrazol-4-yl)benzoic acid

To a solution of methyl 4-(1-(pent-4-yn-1-yl)-1H-pyrazol-4-yl)benzoate (Intermediate 129, 5 g, 18.64 mmol) in tetrahydrofuran (20 mL) and methanol (20 mL) was added lithium hydroxide (1.366 g, 55.91 mmol) in water (10 mL) at 0° C. The reaction mixture was stirred at room temperature for 24 h. Progress of the reaction was monitored by TLC. After completion, the reaction mixture was concentrated in vacuo and water (25 ml) was added to the residue and the pH adjusted to neutralize the solution with 1N HCl. A solid precipitated out, which was filtered, washed with excess of water and dried to obtain 4-(1-(pent-4-yn-1-yl)-1H-pyrazol-4-yl)benzoic acid (Intermediate 130, 3.5 g, 74%) as off-white solid. ¹H NMR (400 MHz, DMSO-d₆) δ ppm 1.94-2.00 (m, 2H), 2.13-2.17 (m, 2H), 2.83 (s, 1H), 4.18 (t, J=6.84 Hz, 2H), 7.67 (d, J=7.90 Hz, 2H), 7.89 (d, J=7.90 Hz, 2H), 7.96 (s, 1H), 8.28 (s, 1H), 12.80 (br s, 1H). Mass spec: m/z 255.1 [M+H]⁺.

### Step 3

### Intermediate 131: 4-(1-(pent-4-yn-1-yl)-1H-pyrazol-4-yl)benzamide

To a solution of 4-(1-(pent-4-yn-1-yl)-1H-pyrazol-4-yl)benzoic acid (Intermediate 130, 3.5 g, 14 mmol) in DMF (30 mL) was added diisopropylethylamine (7.3 g, 55 mmol) and HATU (8.1 g, 21 mmol) followed by ammonium chloride (3.0 g, 55 mmol) at room temperature and stirred for 12 h. Progress of the reaction was monitored by TLC and LCMS. After completion, the reaction mixture was treated with water (100 mL) and extracted with EtOAc (3×100 mL). The organic layer was dried over anhydrous Na₂SO₄ and concentrated in vacuo to afford 4-(1-(pent-4-yn-1-yl)-1H-pyrazol-4-yl)benzamide (Intermediate 131, 2.0 g) as off-white solid. ¹H NMR (400 MHz, DMSO-d₆) δ ppm 1.97-2.02 (m, 2H), 2.14-2.21 (m, 2H), 2.86 (s, 1H), 4.20 (t, J=6.80 Hz, 2H), 7.28 (s, 1H) 7.65 (d, J=7.89 Hz, 2H), 7.86 (d, J=7.89 Hz, 2H), 7.92 (s, 1H), 7.97 (s, 1H), 8.28 (s, 1H). Mass spec: m/z 254.1 [M+H]⁺.

### Step 4

### Intermediate 132: 4-(1-(5-(2-(2,6-dioxo-1-((2-(trimethylsilyl)ethoxy)methyl)piperidin-3-yl)-1-oxoisoindolin-4-yl)pent-4-yn-1-yl)-1H-pyrazol-4-yl)benzamide (8)

To a solution of 4-(1-(pent-4-yn-1-yl)-1H-pyrazol-4-yl)benzamide (Intermediate 131, 600 mg, 2.36 mmol) in DMF (2 mL) was added copper(I) iodide (45 mg, 0.23 mmol), bis(triphenylphosphine)palladium chloride (171 mg, 0.23 mmol) and 3-(4-iodo-1-oxoisoindolin-2-yl)-1-((2-(trimethylsilyl)ethoxy)methyl)piperidine-2,6-dione (Intermediate 8, 1.18 g, 2.36 mmol), followed by triethylamine (12.3 mL, 87.6 mmol). The reaction mixture was purged with argon for 10-15 min. The reaction mixture was stirred at RT for 12 h. Progress of the reaction was monitored by TLC. After completion, water (100 mL) was added and extracted with EtOAc (3×10 mL). The organic layer was dried over anhydrous Na₂SO₄ and concentrated in vacuo. The obtained residue was purified by normal phase MPLC (0-10% EtOAc in heptane) to afford 4-(1-(5-(2-(2,6-dioxopiperidin-3-yl)-1-oxoisoindolin-4-yl)pent-4-yn-1-yl)-1H-pyrazol-4-yl)benzamide (Intermediate 132, 705 mg, 47%) as a white solid. ¹H NMR (400 MHz, DMSO-d₆) δ ppm −0.06 (s, 9H), 0.80-0.86 (m, 2H), 2.03-2.08 (m, 2H), 2.42-2.45 (m, 2H), 2.78-2.80 (m, 2H), 3.04-3.06 (m, 2H), 3.55-3.58 (m, 2H), 4.28 (s, 2H), 4.48-4.53 (m, 2H), 5.04-5.07 (m, 2H), 5.26-5.28 (m, 1H), 7.27 (s, 1H), 7.52-7.54 (m, 1H), 7.62-7.73 (m, 4H), 7.84-7.90 (m, 3H), 7.96 (s, 1H), 8.32 (s, 1H).

### Step 5

### Intermediate 133: tert-butyl 6-(4-(1-(5-(2-(2,6-dioxo-1-((2-(trimethylsilyl)ethoxy)methyl)piperidin-3-yl)-1-oxoisoindolin-4-yl)pent-4-yn-1-yl)-1H-pyrazol-4-yl)benzamido)-2-((R)-1-methylpyrrolidin-2-yl)-1H-pyrrolo[3,2-c]pyridine-1-carboxylate

To a solution of 4-(1-(5-(2-(2,6-dioxopiperidin-3-yl)-1-oxoisoindolin-4-yl)pent-4-yn-1-yl)-1H-pyrazol-4-yl)benzamide (Intermediate 132, 700 mg, 1.12 mmol) in 1,4-dioxane (5 mL) was added cesium carbonate (1.09 g, 3.36 mmol) followed by tert-butyl (R)-6-bromo-2-(1-methylpyrrolidin-2-yl)-1H-pyrrolo[3,2-c]pyridine-1-carboxylate (Intermediate 4, 425 mg, 1.12 mmol) at room temperature. The reaction mixture was purged with argon for 15 min. To this reaction mixture (R)-(+)-2,2′-bis(diphenylphosphino)-1,1′-binaphthyl (71 mg, 0.11 mmol) and dichloro[1,3-bis(2,6-di-3-pentylphenyl)imidazol-2-ylidene](3-chloropyridyl)palladium(II) (93 mg, 0.11 mmol) were added and reaction mixture was again degassed for 5 minutes then heated to 100° C. for 2 h. Progress of the reaction was monitored by TLC and LCMS. After completion of reaction, water (200 mL) and extracted with EtOAc (3×200 mL). The organic layer was dried over anhydrous Na₂SO₄ and concentrated in vacuo. The obtained residue was purified by normal phase MPLC (0-50% EtOAc in heptane) to afford tert-butyl 6-(4-(1-(5-(2-(2,6-dioxo-1-((2-(trimethylsilyl)ethoxy)methyl)piperidin-3-yl)-1-oxoisoindolin-4-yl)pent-4-yn-1-yl)-1H-pyrazol-4-yl)benzamido)-2-((R)-1-methylpyrrolidin-2-yl)-1H-pyrrolo[3,2-c]pyridine-1-carboxylate (Intermediate 133, 700 mg, 67%) as an off-white solid. ¹H NMR (400 MHz, DMSO-d₆) δ ppm −0.06 (s, 9H), 0.74-0.86 (m, 2H), 1.69-1.74 (m, 13H), 2.06-2.14 (m, 4H), 2.35 (s, 5H), 2.75-2.80 (m, 1H), 3.03-3.12 (m, 2H), 3.48-3.53 (m, 2H), 3.90-3.94 (m, 1H), 4.28-4.31 (m, 3H), 4.50-4.53 (m, 1H), 5.00-5.07 (m, 2H) 5.24-5.28 (m, 1H), 6.75 (br s, 1H) 7.50-7.53 (m, Hz, 1H), 7.66-7.74 (m, 4H), 8.01-8.06 (m, 3H), 8.38 (br s, 1H) 8.59 (br s, 1H) 8.94 (br s, 1H) 10.63 (br s, 1H). Mass spec: m/z 925.3 [M+H]⁺.

### Step 6

### Example 21: 4-(1-(5-(2-(2,6-dioxopiperidin-3-yl)-1-oxoisoindolin-4-yl)pent-4-yn-1-yl)-1H-pyrazol-4-yl)-N-(2-((R)-1-methylpyrrolidin-2-yl)-1H-pyrrolo[3,2-c]pyridin-6-yl)benzamide

To a solution of tert-butyl 6-(4-(1-(5-(2-(2,6-dioxo-1-((2-(trimethylsilyl)ethoxy)methyl)piperidin-3-yl)-1-oxoisoindolin-4-yl)pent-4-yn-1-yl)-1H-pyrazol-4-yl)benzamido)-2-((R)-1-methylpyrrolidin-2-yl)-1H-pyrrolo[3,2-c]pyridine-1-carboxylate (Intermediate 133, 700 mg, 0.75 mmol) in acetonitrile (5 mL) was added methanesulfonic acid (1.08 g, 11.34 mmol) at room temperature and reaction mixture was heated at 50° C. for 2 h. The reaction mixture was cooled to room temperature. To this mixture, N,N′-dimethylethylenediamine (396 mg, 4.5 mmol) followed by triethylamine (1.14 g, 11.34 mmol) were added at room temperature and stirred at RT for 3 h. Progress of the reaction was monitored by TLC and LCMS. After completion, the reaction mixture was treated with water (50 mL) and filtered. The crude compound was purified by reverse phase preparative HPLC (Method A) to afford 4-(1-(5-(2-(2,6-dioxopiperidin-3-yl)-1-oxoisoindolin-4-yl)pent-4-yn-1-yl)-1H-pyrazol-4-yl)-N-(2-((R)-1-methylpyrrolidin-2-yl)-1H-pyrrolo[3,2-c]pyridin-6-yl)benzamide (Example 21, 56 mg, 11%) as an off-white solid. ¹H NMR (400 MHz, DMSO-d₆) δ ppm 1.76-1.97 (m, 3H), 1.98-2.10 (m, 1H), 2.12-2.16 (m, 3H), 2.17 (s, 3H), 2.21-2.32 (m, 1H), 2.35-2.47 (m, 2H), 2.52-2.63 (m, 3H), 2.80-3.03 (m, 1H), 3.08-3.24 (m, 1H), 4.25-4.40 (m, 3H), 4.44-4.55 (m, 1H), 5.13-5.14 (m, 1H), 6.39 (s, 1H), 7.49-7.52 (m, 1H), 7.64-7.74 (m, 4H), 8.02 (s, 1H), 8.05-8.06 (m, 2H), 8.20 (s, 1H), 8.38 (s, 1H), 8.50 (s, 1H), 10.38 (s, 1H), 11.36 (s, 1H). Mass spec: m/z 695.5 [M+H]⁺.

### Intermediate 128 was Synthesised Following Scheme 25

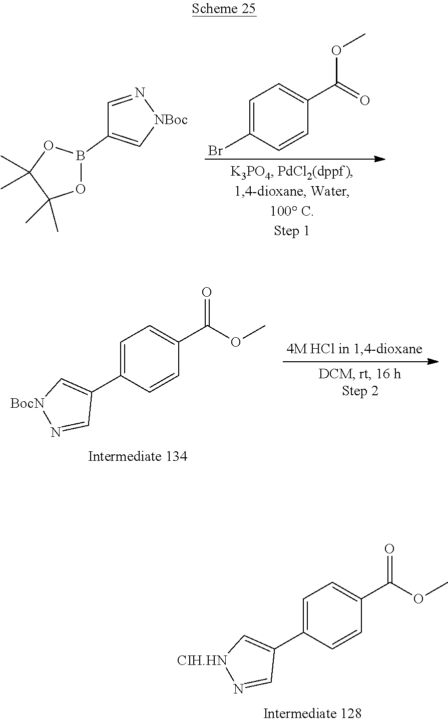

### Step 1

### Intermediate 134: tert-butyl 4-(4-(methoxycarbonyl)phenyl)-1H-pyrazole-1-carboxylate

To a solution of tert-butyl 4-(4,4,5,5-tetramethyl-1,3,2-dioxaborolan-2-yl)-1H-pyrazole-1-carboxylate (CAS No: 552846-17-0, 52.67 g, 179.0 mmol) and methyl 4-bromobenzoate (CAS No: 619-42-1, 35.00 g, 162.8 mmol) in 1,4-dioxane (350 mL) was added potassium phosphate (86.26 g, 406.9 mmol) and water (100 mL). The reaction mixture was purged with argon gas for 15 min then [1,1′-bis(diphenylphosphino)ferrocene]dichloropalladium (II) (5.95 g, 8.14 mmol) was added at room temperature. The reaction mixture was further purged with argon gas for 10 min and then heated at 100° C. for 16 h. The reaction mixture was filtered through celite and the residue was poured into water (1000 mL) and extracted with ethyl acetate (3×500 mL). The combined organic phases were washed with brine solution (2×250 mL), dried over anhydrous Na₂SO₄, filtered, and concentrated in vacuo. The crude material was purified by combi-flash chromatography by eluting with 65% ethyl acetate in heptane to afford tert-butyl 4-(4-(methoxycarbonyl)phenyl)-1H-pyrazole-1-carboxylate (Intermediate 134, 37.0 g, 68.5% yield) as a pale yellow solid. UH NMR (400 MHz, DMSO-d₆) δ ppm 1.61 (s, 9H), 3.86 (s, 3H), 7.89-8.04 (m, 4H), 8.40 (s, 1H), 8.88 (s, 1H).

### Step 2

### Intermediate 128: methyl 4-(1H-pyrazol-4-yl)benzoate hydrochloride

To a solution of tert-butyl 4-(4-(methoxycarbonyl)phenyl)-1H-pyrazole-1-carboxylate (Intermediate 134, 37.00 g, 122.4 mmol) in dichloromethane (100 mL) was added 4M HCl in 1,4-dioxane (50 mL) at 0° C. under nitrogen and the reaction mixture was stirred at room temperature for 16 h. The reaction mixture was concentrated in vacuo to obtain the crude compound. The crude material was triturated with ethyl acetate (2×100 mL) and pentane (2×100 mL) and dried in vacuo to afford methyl 4-(1H-pyrazol-4-yl)benzoate hydrochloride (Intermediate 128, 27 g, (93%) as a white solid. ¹H NMR (400 MHz, DMSO-d₆) δ ppm 3.84 (s, 3H), 7.77 (d, J=8.31 Hz, 2H), 7.93 (d, J=7.82 Hz, 2H), 8.23 (s, 2H). Mass spec: m/z: 203.11 [M+H]⁺.

### Example 22 was Synthesised Following Scheme 26

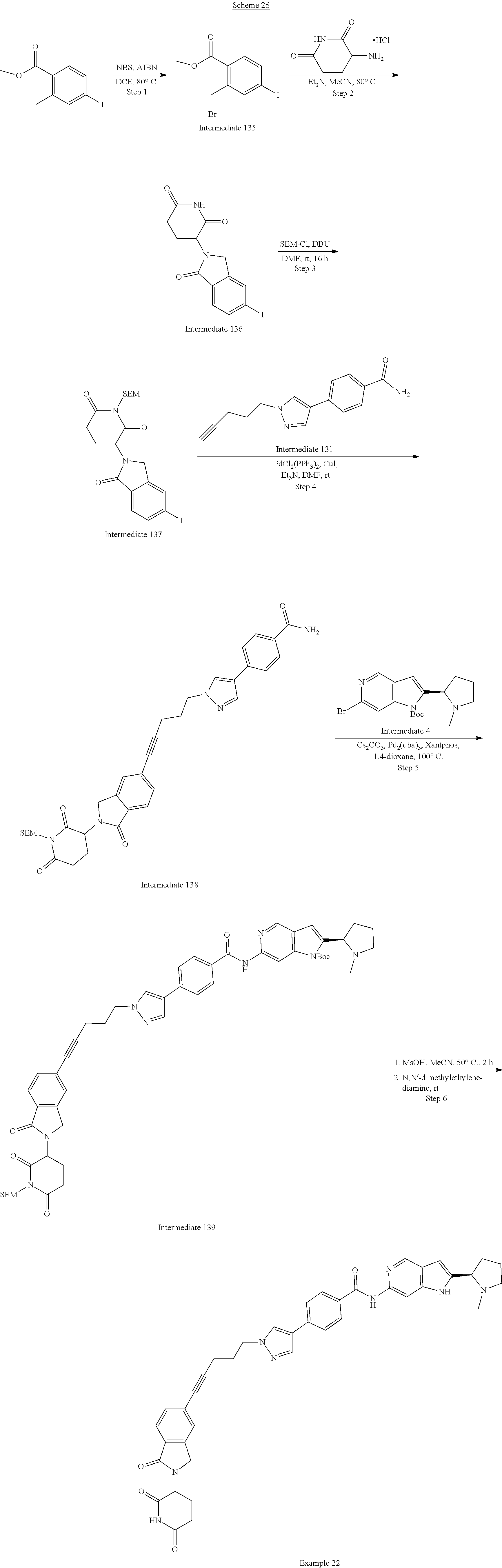

### Step 1

### Intermediate 135: methyl 2-(bromomethyl)-4-iodobenzoate

To solution of methyl 4-iodo-2-methylbenzoate (CAS No: 103440-53-5, 20 g, 72.45 mmol) in dichloroethane (200 mL) were added N-Bromosuccinimide (19.54 g, 108.7 mmol) and AIBN (9.71 g, 57.96 mmol) under nitrogen atmosphere and the reaction mixture was heated at 80° C. for 14 h. The reaction mixture was quenched with water (100 mL) and extracted with dichloromethane (2×100 mL). The organic layers were separated, washed with brine solution (3×200 mL), dried over anhydrous Na₂SO₄, and concentrated in vacuo to afford methyl 2-(bromomethyl)-4-iodobenzoate (Intermediate 135, 15.0 g, 58%) as a brown solid, which was used directly into the next step without further purification. ¹H NMR (400 MHz, DMSO-d₆) δ ppm 3.92 (s, 3H), 4.99 (s, 2H), 7.59-7.68 (m, 1H), 7.80-7.87 (m, 1H), 8.01-8.05 (m, 1H).

### Step 2

### Intermediate 136: 3-(5-iodo-1-oxoisoindolin-2-yl) piperidine-2,6-dione

To a solution of methyl 2-(bromomethyl)-4-iodobenzoate (Intermediate 135, 15 g, 42.25 mmol) in acetonitrile (230 mL) was added 3-aminopiperidine-2,6-dione hydrochloride (CAS No: 24666-56-6, 10.43 g, 63.38 mmol) followed by triethylamine (12.9 g, 126.77 mmol) at 0° C. under a nitrogen atmosphere. The reaction mixture was slowly warmed to room temperature and heated at 80° C. for 14 h. The reaction mixture was quenched with water (100 mL) and extracted with ethyl acetate (2×400 mL). The combined organic phases were washed with brine solution (2×100 mL), dried over anhydrous Na₂SO₄, and concentrated in vacuo to obtain the crude compound which was purified by combi-flash chromatography, by eluting with 1-2% ethyl acetate, in heptane to afford 3-(5-iodo-1-oxoisoindolin-2-yl) piperidine-2,6-dione (Intermediate 136, 12.0 g, 77%) as an off white solid. ¹H NMR (400 MHz, DMSO-d₆) δ ppm 1.98-2.01 (m, 1H), 2.35-2.40 (m, 1H), 2.57-2.61 (m, 1H), 2.86-2.97 (m, 1H), 4.28-4.33 (m, 1H), 4.41-4.46 (m, 1H), 5.07-5.12 (m, 1H), 7.51 (d, J=7.60 Hz, 1H), 7.89 (d, J=7.60 Hz, 1H), 8.06 (s, 1H), 10.99 (br s, 1H). Mass spec: m/z 371.0 [M+H]⁺.

### Step 3

### Intermediate 137: 3-(5-iodo-1-oxoisoindolin-2-yl)-1-((2-(trimethylsilyl)ethoxy)methyl)piperidine-2,6-dione

To a solution of 3-(5-iodo-1-oxoisoindolin-2-yl)piperidine-2,6-dione (Intermediate 136, 2.5 g, 6.8 mmol) in dimethylformamide (20 mL) was added DBU (2.1 g, 14 mmol) followed by 2-(trimethylsilyl)ethoxymethyl chloride (1.8 g, 10 mmol) at 0° C. under a nitrogen atmosphere and stirred the reaction mixture at room temperature for 16 h. The reaction mixture was quenched with water (50 mL) and extracted with ethyl acetate (2×100 mL). The organic layers were washed with brine solution (2×200 mL), separated, dried over anhydrous Na₂SO₄, and concentrated in vacuo to obtain crude compound. The crude material was purified by combi-flash chromatography, by eluting with 2% MeOH in DCM, to afford 3-(5-iodo-1-oxoisoindolin-2-yl)-1-((2-(trimethylsilyl)ethoxy)methyl)piperidine-2,6-dione (Intermediate 137, 0.85 g, 25%) as a dark blue solid. ¹H NMR (400 MHz, DMSO-d₆) δ ppm −0.02 (s, 9H), 0.81-0.85 (m, 2H), 2.04-2.06 (m, 1H), 2.33-2.40 (m, 1H), 2.76-2.81 (m, 1H), 3.02-3.09 (m, 1H), 3.51-3.53 (m, 2H), 4.26-4.31 (m, 1H), 4.44-4.48 (m, 1H), 5.01-5.08 (m, 2H), 5.19-5.23 (m, 1H), 7.53 (d, J=7.60 Hz, 1H), 7.90 (d, J=7.60 Hz, 1H), 8.07 (s, 1H).

### Step 4

### Intermediate 138: 4-(1-(5-(2-(2,6-dioxo-1-((2-(trimethylsilyl)ethoxy)methyl)piperidin-3-yl)-1-oxoisoindolin-5-yl) pent-4-yn-1-yl)-1H-pyrazol-4-yl)benzamide

To a solution of 4-(1-(pent-4-yn-1-yl)-1H-pyrazol-4-yl)benzamide (Intermediate 131, 0.45 g, 1.77 mmol) in dimethylformamide (10.0 mL) was added 3-(5-iodo-1-oxoisoindolin-2-yl)-1-((2-(trimethylsilyl) ethoxy)methyl)piperidine-2,6-dione (Intermediate 137, 0.88 g, 1.77 mmol) followed by triethylamine (6.32 g, 62.18 mmol). The reaction mixture was purged with argon gas for 15 min then copper(I) iodide (0.03 g, 0.17 mmol) followed by PdCl₂(PPh₃)₂ (0.13 g, 0.17 mmol) were added and the reaction was stirred for 2 h at room temperature. The reaction mixture was diluted with water (100 mL) and extracted with ethyl acetate (3×100 mL). The organic layers were separated, dried over anhydrous Na₂SO₄, and concentrated in vacuo to obtain crude compound. The crude material was purified by combi-flash chromatography, by eluting with 100% ethyl acetate in heptane, to afford 4-(1-(5-(2-(2,6-dioxo-1-((2-(trimethylsilyl)ethoxy)methyl)piperidin-3-yl)-1-oxoisoindolin-5-yl)pent-4-yn-1-yl)-1H-pyrazol-4-yl)benzamide (Intermediate 138, 0.53 g, 59%) as a yellow solid. ¹H NMR (400 MHz, DMSO-d₆) δ ppm −0.02 (s, 9H), 0.81-0.86 (m, 2H), 2.01-2.14 (m, 3H), 2.33-2.42 (m, 1H), 2.71-2.73 (m, 2H), 2.77-2.81 (m, 1H), 3.02-3.11 (m, 1H), 3.49-3.55 (m, 2H), 4.26-4.31 (m, 3H), 4.38-4.43 (m, 1H), 5.01-5.08 (m, 2H), 5.20-5.25 (m, 1H), 7.31 (br s, 1H), 7.53 (d, J=8.40 Hz, 1H), 7.62-7.69 (m, 4H), 7.86 (d, J=8.40 Hz, 2H), 7.93-7.95 (m, 1H), 8.00 (s, 1H), 8.35 (br s, 1H). Mass spec: m/z: 626.0 [M+H]⁺.

### Step 5

### Intermediate 139: tert-butyl 6-(4-(1-(5-(2-(2,6-dioxo-1-((2-(trimethylsilyl)ethoxy)methyl)piperidin-3-yl)-1-oxoisoindolin-5-yl)pent-4-yn-1-yl)-1H-pyrazol-4-yl)benzamido)-2-((R)-1-methylpyrrolidin-2-yl)-1H-pyrrolo[3,2-c]pyridine-1-carboxylate

To a solution of 4-(1-(5-(2-(2,6-dioxo-1-((2-(trimethylsilyl)ethoxy)methyl)piperidin-3-yl)-1-oxoisoindolin-5-yl)pent-4-yn-1-yl)-1H-pyrazol-4-yl)benzamide (Intermediate 138, 0.30 g, 0.47 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 0.18 g, 0.47 mmol) followed by cesium carbonate (0.31 g, 0.96 mmol) at room temperature. The reaction mixture was purged with argon gas for 15 min then Pd₂(dba)₃ (0.045 g, 0.047 mmol) and Xantphos (0.085 g, 0.14 mmol) were added. The reaction mixture was further purged with argon gas for 10 min and heated at 100° C. for 2 h. The reaction mixture was then concentrated in vacuo to obtain crude compound. The crude material purified by combi-flash chromatography, by eluting with 80% ethyl acetate in heptane, to afford tert-butyl 6-(4-(1-(5-(2-(2,6-dioxo-1-((2-(trimethylsilyl)ethoxy)methyl)piperidin-3-yl)-1-oxoisoindolin-5-yl)pent-4-yn-1-yl)-1H-pyrazol-4-yl)benzamido)-2-((R)-1-methylpyrrolidin-2-yl)-1H-pyrrolo[3,2-c]pyridine-1-carboxylate (Intermediate 139, 0.31 g, 70%) as a yellow solid. ¹H NMR (400 MHz, DMSO-d₆) δ ppm −0.03 (s, 9H), 0.79-0.85 (m, 2H), 1.61-1.66 (m, 2H), 1.70 (s, 9H), 1.68-1.70 (m, 2H), 1.74-1.77 (m, 1H), 2.01-2.07 (m, 2H), 2.31-2.33 (m, 1H), 2.36 (s, 3H), 2.36-2.38 (m, 2H), 2.52-2.55 (m, 2H), 2.72-2.76 (m, 1H), 3.03-3.07 (m, 1H), 3.37-3.51 (m, 2H), 3.90-3.94 (m, 1H), 4.18-4.22 (m, 1H), 4.28-4.32 (m, 2H), 4.36-4.40 (m, 1H), 4.95-4.99 (m, 1H), 5.03-5.06 (m, 1H), 5.19-5.23 (m, 1H), 6.75 (s, 1H), 7.52 (d, J=7.64 Hz, 1H), 7.58 (s, 1H), 7.68 (d, J=7.64 Hz, 1H), 7.70-7.73 (m, 2H), 8.03-8.05 (m, 2H), 8.07 (s, 1H), 8.41 (s, 1H), 8.59 (s, 1H), 8.95 (s, 1H), 10.65 (s, 1H). Mass spec: m/z: 924.8 [M−H]⁻.

### Step 6

### Example 22: 4-(1-(5-(2-(2,6-dioxopiperidin-3-yl)-1-oxoisoindolin-5-yl)pent-4-yn-1-yl)-1H-pyrazol-4-yl)-N-(2-((R)-1-methylpyrrolidin-2-yl)-1H-pyrrolo[3,2-c]pyridin-6-yl)benzamide

To a solution of tert-butyl 6-(4-(1-(5-(2-(2,6-dioxo-1-((2-(trimethylsilyl)ethoxy)methyl)piperidin-3-yl)-1-oxoisoindolin-5-yl)pent-4-yn-1-yl)-1H-pyrazol-4-yl)benzamido)-2-((R)-1-methylpyrrolidin-2-yl)-1H-pyrrolo[3,2-c]pyridine-1-carboxylate (Intermediate 139, 0.40 g, 0.43 mmol) in acetonitrile (10 mL) was added methanesulfonic acid (0.28 mL, 4.32 mmol) at room temperature and the reaction mixture was heated at 50° C. for 2 h. After cooling to room temperature, N,N′-dimethylethylenediamine (0.21 g, 2.162 mmol) followed by triethylamine (0.87 g, 8.64 mmol) was added the reaction was stirred for 3 h at room temperature. The reaction mixture was poured into ice cold water (20 mL), the resulting precipitate was filtered and dried under vacuum to obtain crude compound. The crude material was purified by preparative HPLC (Method A) to afford 4-(1-(5-(2-(2,6-dioxopiperidin-3-yl)-1-oxoisoindolin-5-yl)pent-4-yn-1-yl)-1H-pyrazol-4-yl)-N-(2-((R)-1-methylpyrrolidin-2-yl)-1H-pyrrolo[3,2-c]pyridin-6-yl)benzamide (Example 22, 0.065 g, 22%) as an off white solid. ¹H NMR (400 MHz, DMSO-d₆) δ ppm 1.78-1.95 (m, 3H), 1.97-2.03 (m, 1H), 2.11-2.16 (m, 3H), 2.17 (s, 3H), 2.23-2.30 (m, 1H), 2.36-2.41 (m, 1H), 2.52-2.54 (m, 2H), 2.57-2.62 (m, 1H), 2.85-2.94 (m, 2H), 3.12-3.16 (m, 1H), 4.27-4.32 (m, 3H), 4.37-4.41 (m, 1H), 5.08-5.13 (m, 1H), 6.40 (s, 1H), 7.52 (d, J=7.83 Hz, 1H), 7.61 (s, 1H), 7.65-7.72 (m, 3H), 8.01-8.08 (m, 3H), 8.20 (s, 1H), 8.39 (s, 1H), 8.51 (s, 1H), 10.38 (s, 1H), 11.06 (s, 1H), 11.36 (s, 1H). Mass spec: m/z: 694.8 [M+H]⁺.

### Example 23 was Synthesised Following Scheme 27

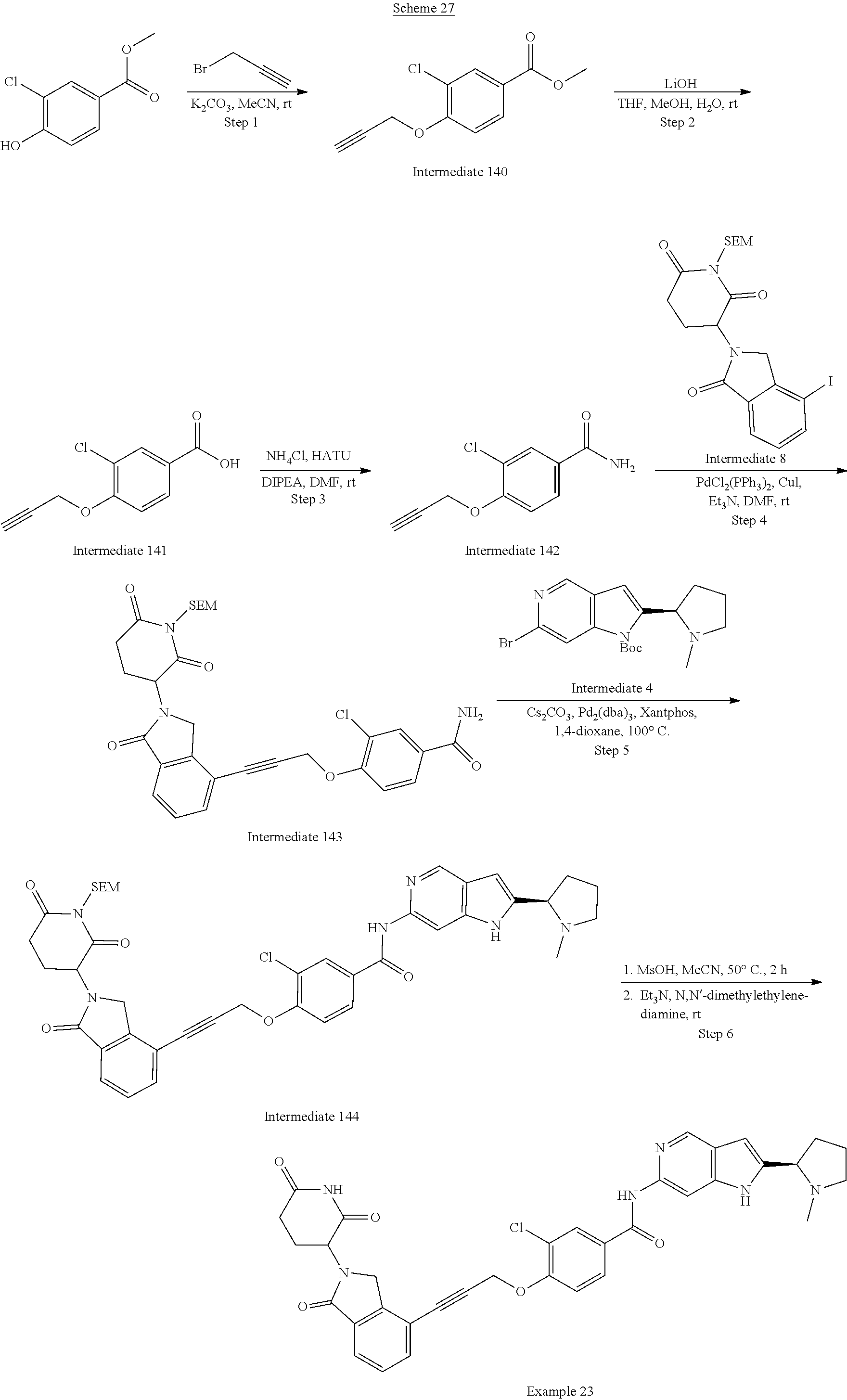

### Step 1

### Intermediate 140: methyl 3-chloro-4-(prop-2-yn-1-yloxy)benzoate

To solution of methyl 3-chloro-4-hydroxy-benzoate (CAS No: 3964-57-6, 10 g, 53.59 mmol) in acetonitrile (100 mL) was added potassium carbonate (8.87 g, 64.31 mmol) followed by 3-bromoprop-1-yne (CAS No: 106-96-7, 7.65 g, 64.31 mmol) at room temperature and the reaction mixture was stirred for 6 h. The reaction mixture was then diluted with water (1000 mL) and extracted with dichloromethane (3×500 mL). The organic layers were separated, dried over anhydrous Na₂SO₄, filtered, and concentrated in vacuo. The crude material was purified by combi-flash chromatography, by eluting with 90% ethyl acetate in heptane, to afford methyl 3-chloro-4-(prop-2-yn-1-yloxy)benzoate (Intermediate 140, 11 g, 83%) as a white solid. ¹H NMR (400 MHz, DMSO-d₆) δ ppm 3.69 (s, 1H), 3.84 (s, 3H), 5.04 (s, 2H), 7.30-7.35 (m, 1H), 7.91-7.95 (m, 2H).

### Step 2

### Intermediate 141: 3-chloro-4-(prop-2-yn-1-yloxy)benzoic acid

To a solution of methyl 3-chloro-4-(prop-2-yn-1-yloxy)benzoate (Intermediate 140, 11 g, 48.96 mmol) in tetrahydrofuran (50 mL) was added MeOH (50 mL) and water (25 mL) followed by lithium hydroxide (4.78 g, 195.87 mmol) at 0° C. and the reaction mixture was stirred at room temperature for 6 h. The reaction mixture was then diluted with water (5 mL) and concentrated in vacuo. The pH of the aqueous layer was adjusted to pH~4 by dropwise addition 0.5 N HCl. The resulting suspension was extracted with ethyl acetate (2×250 mL). The organic layers were separated, dried over anhydrous Na₂SO₄, filtered, and concentrated under reduced pressure to afford 3-chloro-4-(prop-2-yn-1-yloxy)benzoic acid (Intermediate 141, 9.60 g, 93%) as an off-white solid, which was used for the next step without further purification. ¹H NMR (400 MHz, DMSO-d₆) δ ppm 3.68 (s, 1H), 5.02 (s, 2H), 7.29-7.32 (m, 1H), 7.89-7.92 (m, 2H). Mass spec: m/z: 208.9 [M−H]⁻.

### Step 3

### Intermediate 142: 3-chloro-4-(prop-2-yn-1-yloxy)benzamide

To a solution of 3-chloro-4-(prop-2-yn-1-yloxy)benzoic acid (Intermediate 141, 10.5 g, 49.9 mmol) in dimethylformamide (100 mL) was added HATU (29.9 g, 74.8 mmol) and stirred for 10 min at rt. To the reaction mixture was added ammonium chloride (8.72 mL, 249 mmol) followed by DIPEA (19.7 g, 150 mmol) at room temperature and the reaction mixture was stirred for 16 h. The reaction mixture was then diluted with water (500 mL) and extracted with ethyl acetate (3×250 mL). The organic layers were separated, dried over anhydrous Na₂SO₄, filtered, and concentrated in vacuo to afford 3-chloro-4-(prop-2-yn-1-yloxy)benzamide (Intermediate 142, 8.00 g, 76%) as a white solid, which was used for the next step without further purification. ¹H NMR (400 MHz, DMSO-d₆) δ ppm 3.70 (s, 1H), 5.00 (s, 2H), 7.25-7.28 (m, 1H), 7.36 (br s, 1H), 7.84-7.92 (m, 2H), 7.96 (br s, 1H). Mass spec: m/z 209.9 [M+H]⁺.

### Step 4

### Intermediate 143: 3-chloro-4-((3-(2-(2,6-dioxo-1-((2-(trimethylsilyl)ethoxy)methyl)piperidin-3-yl)-1-oxoisoindolin-4-yl)prop-2-yn-1-yl)oxy)benzamide

To a solution of 3-chloro-4-(prop-2-yn-1-yloxy)benzamide (Intermediate 142, 1 g, 4.77 mmol) and 3-(4-iodo-1-oxoisoindolin-2-yl)-1-(2-trimethylsilylethoxymethyl)piperidine-2,6-dione (Intermediate 8, 2.38 g, 4.77 mmol) in dimethylformamide (10 mL) was added triethylamine (24.1 mL, 171.73 mmol) at room temperature. The reaction mixture was purged with argon gas for 15 min then PdCl₂(PPh₃)₂ (0.34 g, 0.477 mmol) and copper (I) iodide (0.093 g, 0.477 mmol) were added at room temperature. The reaction mixture was further purged with argon gas for 10 min and stirred at room temperature for 2 h. The reaction mixture was concentrated in vacuo to obtain crude compound. This crude material was purified by combi-flash chromatography, by eluting with 80% ethyl acetate in heptane, to afford 3-chloro-4-((3-(2-(2,6-dioxo-1-((2-(trimethylsilyl)ethoxy)methyl)piperidin-3-yl)-1-oxoisoindolin-4-yl) prop-2-yn-1-yl)oxy)benzamide (Intermediate 143, 1.6 g, 58%) as a yellow solid. ¹H NMR (400 MHz, DMSO-d₆) δ ppm −0.03 (s, 9H), 0.81-0.88 (m, 2H), 2.03-2.09 (m, 1H), 2.27-236 (m, 1H), 2.79-2.83 (m, 3H), 3.03-3.12 (m, 1H), 3.50-3.55 (m, 2H), 4.13-4.18 (m, 1H), 4.32-4.36 (m, 1H), 5.03-5.09 (m, 1H), 5.31 (s, 2H), 7.35 (br s, 1H), 7.40-7.55 (m, 1H), 7.58-7.71 (m, 1H), 7.72-7.78 (m, 1H), 7.80-7.87 (m, 1H), 7.89-7.93 (m, 1H), 7.95-7.98 (m, 1H), 8.00 (br s, 1H). Mass spec: m/z 579.9 [M−H]⁻.

### Step 5

### Intermediate 144: tert-butyl 6-(3-chloro-4-((3-(2-(2,6-dioxo-1-((2-(trimethylsilyl)ethoxy)methyl)piperidin-3-yl)-1-oxoisoindolin-4-yl)prop-2-yn-1-yl)oxy)benzamido)-2-((R)-1-methylpyrrolidin-2-yl)-1H-pyrrolo[3,2-c]pyridine-1-carboxylate

To a solution of 3-chloro-4-((3-(2-(2,6-dioxo-1-((2-(trimethylsilyl)ethoxy)methyl)piperidin-3-yl)-1-oxoisoindolin-4-yl) prop-2-yn-1-yl)oxy)benzamide (Intermediate 143, 1.22 g, 2.10 mmol) and tert-butyl (R)-6-bromo-2-(1-methylpyrrolidin-2-yl)-1H-pyrrolo[3,2-c]pyridine-1-carboxylate (Intermediate 4, 1.00 g, 2.63 mmol) in 1,4-dioxane (20 mL) was added cesium carbonate (2.57 g, 7.88 mmol). The reaction mixture was purged with argon gas for 15 min then Xantphos (0.47 g, 0.788 mmol) and Pd₂(dba)₃ (0.37 g, 0.39 mmol) were added at room temperature. The reaction mixture was further purged with argon gas for another 10 min and heated at 100° C. for 2 h. The reaction mixture was then concentrated in vacuo. The resulting crude material was purified by combi-flash chromatography, by eluting with 6% MeOH in DCM, to afford tert-butyl 6-(3-chloro-4-((3-(2-(2,6-dioxo-1-((2-(trimethylsilyl)ethoxy)methyl)piperidin-3-yl)-1-oxoisoindolin-4-yl)prop-2-yn-1-yl)oxy)benzamido)-2-((R)-1-methylpyrrolidin-2-yl)-1H-pyrrolo[3,2-c]pyridine-1-carboxylate (Intermediate 144, 0.34 g, 15%) as a yellow solid. ¹H NMR (400 MHz, DMSO-d₆) δ ppm −0.08 (s, 9H), 0.71-0.87 (m, 2H), 1.07-1.11 (m, 2H), 1.51-1.59 (m, 2H), 1.70 (s, 9H), 2.23-2.32 (m, 2H), 2.35 (s, 3H), 2.37-2.42 (m, 2H), 3.01-3.17 (m, 2H), 3.90-3.94 (m, 2H), 4.14-4.16 (m, 1H), 4.33-4.36 (m, 2H), 4.96-5.02 (m, 2H), 5.21-5.26 (m, 1H), 5.35 (s, 2H), 6.75 (s, 1H), 7.42-7.48 (m, 1H), 7.54-7.60 (m, 1H), 7.74-7.77 (m, 1H), 7.79-7.85 (m, 1H), 8.08-8.10 (m, 1H), 8.15-8.20 (m, 1H), 8.59 (s, 1H), 8.91 (s, 1H), 10.73 (br s, 1H). Mass spec: m/z 881.0 [M+H]⁺.

### Step 6

### Example 23: 3-chloro-4-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)benzamide

To a solution of tert-butyl 6-(3-chloro-4-((3-(2-(2,6-dioxo-1-((2-(trimethylsilyl)ethoxy)methyl)piperidin-3-yl)-1-oxoisoindolin-4-yl)prop-2-yn-1-yl)oxy)benzamido)-2-((R)-1-methylpyrrolidin-2-yl)-1H-pyrrolo[3,2-c]pyridine-1-carboxylate (Intermediate 144, 0.30 g, 0.34 mmol) in acetonitrile (25 mL) was added methanesulfonic acid (0.22 mL, 3.403 mmol) at room temperature and the reaction mixture was heated at 50° C. for 2 h. The reaction mixture was cooled to room temperature then N,N′-dimethylethylenediamine (0.20 mL, 1.70 mmol) followed by triethylamine (0.95 mL, 6.80 mmol) were added and the reaction stirred at room temperature and for 3 h. The reaction mixture was concentrated in vacuo and with water (20 mL) added resulting in s solid precipitating. The solid was filtered and and purified by preparative HPLC (Method A) to afford 3-chloro-4-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)benzamide (Example 23, 0.046 g, 21%) as an off white solid. ¹H NMR (400 MHz, DMSO-D₆) δ ppm 1.77-1.94 (m, 3H), 1.98-2.04 (m, 1H), 2.11-2.14 (m, 1H), 2.17 (s, 3H), 2.22-2.30 (m, 1H), 2.38-2.43 (m, 1H), 2.59-2.64 (m, 1H), 2.86-2.96 (d, 1H), 3.12-3.16 (i, 1H), 3.33-3.35 (m, 1H), 4.28-4.33 (i, 1H), 4.39-4.43 (m, 1H), 5.13-5.18 (i, 1H), 5.36 (s, 2H), 6.40 (s, 1H), 7.47-7.51 (i, 1H), 7.54-7.59 (i, 1H), 7.72-7.76 (i, 1H), 7.77-7.80 (m, 1H), 8.08-8.12 (m, 1H), 8.18 (s, 2H), 8.50 (s, 1H), 10.47 (br s, 1H), 11.22 (br s, 1H), 11.39 (br s, 1H). Mass spec: m/z 651.0 [M+H]⁺.

Examples 24-25 in Table 1 were synthesised in an analogous manner to Example 23, following Step 6 from Scheme 27 by deprotection of the appropriate Intermediate 145 or 146

TABLE 1

| | | | ¹H NMR (400 MHz, | Mass |
|---|---|---|---|---|
| Example | Example Structure and Name | Intermediate | DMSO-d6) δ ppm | spec m/z |
| 24 | 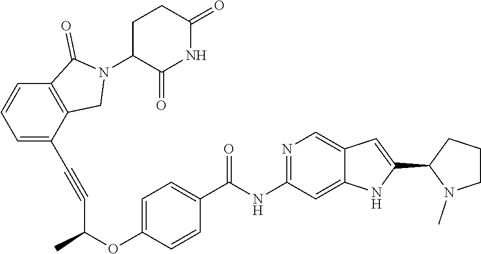 [SMILES: C[C@@H](C#Cc1cccc2c1CN(C1CCC(=O)NC1=O)C2=O)Oc1ccc(C(=O)Nc2cc3[nH]c([C@H]4CCCN4C)cc3cn2)cc1] | 145 | 1.73 (d, J = 6.40 Hz, 3H), 1.78-2.01 (m, 4H), 2.10-2.15 (m, 1H), 2.17 (s, 3H), 2.23-2.30 (m, 1H), 2.31-2.36 (m, 1H), 2.59-2.64 (m, 2H), 2.86-2.97 (m, 1H), 3.10-3.18 (m, 1H), 4.19-4.23 (m, 1H), 4.31-4.36 (m, 1H), 5.16-5.21 (m, 1H), 5.52-5.57 (m, 1H), 6.41 (s, 1H), 7.19 (d, J = 8.80 Hz, 2H), 7.53 (m, 1H), 7.67 | 631.0 [M + H]+ |
| | | | (d, J = 6.80 Hz, 1H), | |
| | 4-(((2S)-4-(2-(2,6- | | 7.75 (d, J = 6.80 Hz, | |
| | dioxopiperidin-3-yl)-1- | | 1H), 8.04 (d, J = 8.80 | |
| | oxoisoindolin-4-yl)but-3-yn-2- | | Hz, 2H), 8.21 (s, | |
| | yl)oxy)-N-(2-((R)-1- | | 1H), 8.49 (s, 1H), | |
| | methylpyrrolidin-2-yl)-1H- | | 10.22 (s, 1H), 11.39 | |
| | pyrrolo[3,2-c]pyridin-6- | | (s, 1H), 11.54 (s, | |
| | yl)benzamide | | 1H) | |
| 25 | 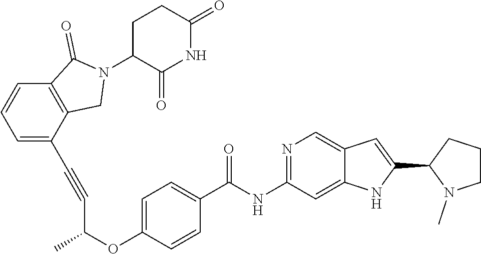 [SMILES: C[C@H](C#Cc1cccc2c1CN(C1CCC(=O)NC1=O)C2=O)Oc1ccc(C(=O)Nc2cc3[nH]c([C@H]4CCCN4C)cc3cn2)cc1] | 146 | 1.72 (d, J = 5.60 Hz, 3H), 1.78-1.92 (m, 3H), 1.94-2.00 (m, 2H), 2.12-2.14 (m, 1H), 2.16 (s, 3H), 2.23-2.29 (m, 1H), 2.33-2.39 (m, 1H), 2.59-2.64 (m, 1H), 2.87-2.96 (m, 1H), 3.12-3.16 (m, 1H), 4.18-4.23 (m, 1H), 4.31-4.37 (m, 1H), 5.16-5.21 (m, 1H), 5.52-5.57 (m, 1H), 6.41 (s, 1H), 7.19 (d, J = 9.00 Hz, 2H), | 631.0 [M + H]+ |
| | | | 7.53 (t, J = 7.20 Hz, | |
| | 4-(((2R)-4-(2-(2,6- | | 1H), 7.67 (d, J = 7.20 | |
| | dioxopiperidin-3-yl)-1- | | Hz, 1H), 7.75 (d, | |
| | oxoisoindolin-4-yl)but-3-yn-2- | | J = 7.20 Hz, 1H), | |
| | yl)oxy)-N-(2-((R)-1- | | 8.04 (d, J = 9.00 Hz, | |
| | methylpyrrolidin-2-yl)-1H- | | 2H), 8.21 (s, 1H), | |
| | pyrrolo[3,2-c]pyridin-6- | | 8.49 (s, 1H), 10.23 | |
| | yl)benzamide | | (s, 1H), 11.40 (s, | |
| | | | 1H), 11.57 (s, 1H) | |

Intermediates 145-146 shown in Table 2 were prepared in an analogous manner to Intermediate 144, following Step 5 from Scheme 27, using Intermediate 4 and the appropriate amide Intermediates 147-148.

TABLE 2

| Inter- | Intermediate Structure and | Amide | ¹H NMR (400 MHz, | Mass |
|---|---|---|---|---|
| mediate | Name | Intermediate | DMSO-d6) δ ppm | spec m/z |
| 145 | 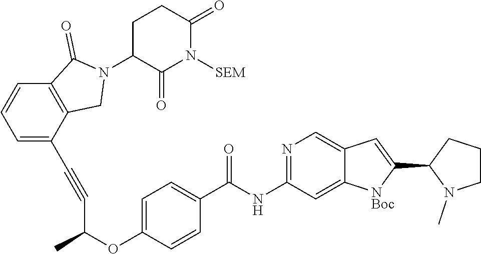 [SMILES: C[C@@H](C#Cc1cccc2c1CN(C1CCC(=O)N(COCC[Si](C)(C)C)C1=O)C2=O)Oc1ccc(C(=O)Nc2cc3c(cn2)cc([C@H]2CCCN2C)n3C(=O)OC(C)(C)C)cc1] | 147 | −0.07 (s, 9H), 0.72- 0.79 (m, 2H), 1.57- 1.67 (m, 4H), 1.71 (s, 9H), 1.73 (m, 3H), 1.98-2.07 (m, 1H), 2.23-2.32 (m, 3H), 2.36 (s, 3H), 2.71- 2.81 (m, 1H), 2.98- 3.07 (m, 1H), 3.41- 3.46 (m, 2H), 3.91- 3.97 (m, 1H), 4.01- 4.09 (m, 1H), 4.17- 4.25 (m, 1H), 4.92- 5.01 (m, 2H), 5.16- 5.23 (m, 1H), 5.55- 5.60 (m, 1H), 6.75 (s, | 861.1 [M + H]⁺ |
| | | | 1H), 7.17-7.20 (m, | |
| | tert-butyl 6-(4-(((2S)-4-(2- | | 2H), 7.53-7.57 (m, | |
| | (2,6-dioxo-1-((2- | | 1H), 7.68 (d, J = 7.80 | |
| | (trimethylsilyl)ethoxy)methyl) | | Hz, 1H), 7.76 (d, | |
| | piperidin-3-yl)-1- | | J = 7.80 Hz, 1H), 8.06- | |
| | oxoisoindolin-4-yl)but-3-yn- | | 8.09 (m, 2H), 8.58 (s, | |
| | 2-yl)oxy)benzamido)-2-((R)- | | 1H), 8.94 (s, 1H), | |
| | 1-methylpyrrolidin-2-yl)- | | 10.53 (br s, 1H) | |
| | 1H-pyrrolo[3,2-c]pyridine-1- | | | |
| | carboxylate | | | |
| 146 | 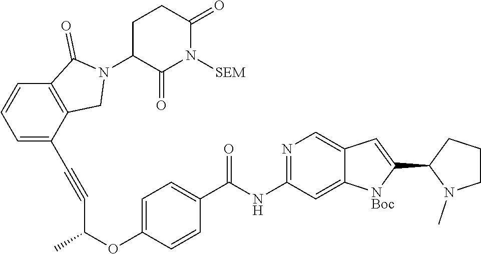 [SMILES: C[C@H](C#Cc1cccc2c1CN(C1CCC(=O)N(COCC[Si](C)(C)C)C1=O)C2=O)Oc1ccc(C(=O)Nc2cc3c(cn2)cc([C@H]2CCCN2C)n3C(=O)OC(C)(C)C)cc1] | 148 | −0.07 (s, 9H), 0.74- 0.77 (m, 2H), 1.57- 1.71 (m, 1H), 1.70 (s, 9H), 1.72 (s, 3H), 1.98-2.07 (m, 2H), 2.27-2.32 (m, 3H), 2.35 (s, 3H), 2.70- 2.78 (m, 1H), 2.97- 3.06 (m, 1H), 3.11- 3.13 (m, 1H), 3.41- 3.46 (m, 2H), 3.92- 4.09 (m, 3H), 4.17- 4.25 (m, 1H), 4.95- 4.99 (m, 2H), 5.17- 5.20 (m, 1H), 5.55- 5.57 (m, 1H), 6.74 (s, | 861.7 [M + H]⁺ |
| | | | 1H), 7.18 (d, J = 7.20 | |
| | tert-butyl 6-(4-(((2R)-4-(2- | | Hz, 2H), 7.54 (t, | |
| | (2,6-dioxo-1-((2- | | J = 7.20 Hz, 1H), 7.67 | |
| | (trimethylsilyl)ethoxy)methyl) | | (d, J = 7.20 Hz, 1H), | |
| | piperidin-3-yl)-1- | | 7.75 (d, J = 7.20 Hz, | |
| | oxoisoindolin-4-yl)but-3-yn- | | 1H), 8.07 (d, J = 7.20 | |
| | 2-yl)oxy)benzamido)-2-((R)- | | Hz, 2H), 8.57 (s, 1H), | |
| | 1-methylpyrrolidin-2-yl)- | | 8.92 (s, 1H), 10.52 | |
| | 1H-pyrrolo[3,2-c]pyridine-1- | | (br s, 1H) | |
| | carboxylate | | | |

Intermediates 147-148 shown in Table 3 were synthesised in an analogous manner to Intermediate 143, following Step 4 from Scheme 27, using Intermediate 8 and the appropriate alkyne Intermediates 149-150.

TABLE 3

| Inter- | Intermediate Structure and | Alkyne | ¹H NMR (400 MHz, | Mass |
|---|---|---|---|---|
| mediate | Name | Intermediate | DMSO-d6) δ ppm | spec m/z |
| 147 | 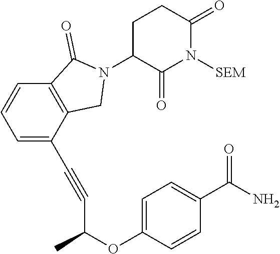 [SMILES: C[C@@H](C#Cc1cccc2c1CN(C1CCC(=O)N(COCC[Si](C)(C)C)C1=O)C2=O)Oc1ccc(C(N)=O)cc1] | 149 | −0.02 (s, 9H), 0.84- 1.86 (m, 2H), 1.70 (d, J = 5.81 Hz, 3H), 1.99- 2.03 (m, 1H), 2.22- 2.30 (m, 1H), 2.73- 2.89 (m, 1H), 3.03- 3.10 (m, 1H), 3.50- 3.61 (m, 2H), 3.94- 4.22 (m, 2H), 5.04- 5.12 (m, 2H), 5.19- 5.22 (m, 1H), 5.50- 5.53 (m, 1H), 7.12 (d, J = 8.00 Hz, 2H), 7.19 (br s, 1H), 7.52-7.56 (m, 1H), 7.65-7.67 (m, 1H), 7.75-7.77 | 560.0 [M − H]⁻ |
| | | | (m, 1H), 7.82 (br s, | |
| | 4-(((2S)-4-(2-(2,6-dioxo-1- | | 1H), 7.87 (d, J = 8.00 | |
| | ((2- | | Hz, 2H) | |
| | (trimethylsilyl)ethoxy)methyl) | | | |
| | piperidin-3-yl)-1- | | | |
| | oxoisoindolin-4-yl)but-3- | | | |
| | yn-2-yl)oxy)benzamide | | | |
| 148 | 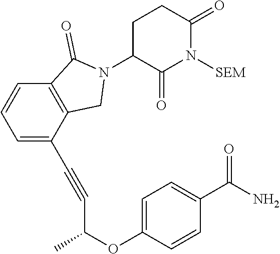 [SMILES: C[C@H](C#Cc1cccc2c1CN(C1CCC(=O)N(COCC[Si](C)(C)C)C1=O)C2=O)Oc1ccc(C(N)=O)cc1] | 150 | −0.02 (s, 9H), 0.84- 0.86 (m, 2H), 1.69 (d, J = 6.00 Hz, 3H), 1.99- 2.03 (m, 1H), 2.24- 2.35 (m, 1H), 2.73- 2.89 (m, 1H), 3.03- 3.10 (m, 1H), 3.50- 3.61 (m, 2H), 3.98- 4.22 (m, 2H), 5.04- 5.12 (m, 2H), 5.19- 5.22 (m, 1H), 5.50- 5.53 (m, 1H), 7.12 (d, J = 7.60 Hz, 2H), 7.18 (br s, 1H), 7.54 (t, J = 7.20 Hz, 1H), 7.66 (d, J = 7.20 Hz, 1H), | 560.0 [M − H]⁻ |
| | | | 7.75 (d, J = 7.20 Hz, | |
| | 4-(((2R)-4-(2-(2,6-dioxo-1- | | 1H), 7.81 (br s, 1H), | |
| | ((2- | | 7.86 (d, J = 7.60 Hz, | |
| | (trimethylsilyl)ethoxy)methyl) | | 2H) | |
| | piperidin-3-yl)-1- | | | |
| | oxoisoindolin-4-yl)but-3-yn- | | | |
| | 2-yl)oxy)benzamide | | | |

Intermediates 149-150 shown in Table 4 were prepared in an analogous manner to Intermediate 142, following Step 3 from Scheme 27, using the appropriate acid Intermediates 151-152.

TABLE 4

| Inter- | Intermediate Structure and | Acid | ¹H NMR (400 MHz, | Mass |
|---|---|---|---|---|
| mediate | Name | Intermediate | DMSO-d6) δ ppm | spec m/z |
| 149 | 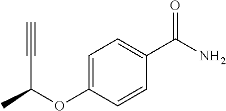 | 151 | 1.56 (d, J = 6.00 Hz, 3H), 3.55 (s, 1H), 5.15-5.21 (m, 1H), 7.02 (d, J = 8.00 Hz, 2H), 7.18 (br s, 1H), 7.82-7.85 (m, 3H) | 190.0 [M + H]⁺ |
| | (S)-4-(but-3-yn-2-yloxy) | | | |
| | benzamide | | | |
| 150 | 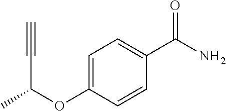 | 152 | 1.56 (d, J = 6.00 Hz, 3H), 3.55 (s, 1H), 5.18-5.20 (m, 1H), 7.02 (d, J = 7.20 Hz, 2H), 7.19 (br s, 2H), 7.82-7.85 (m, 3H) | 190.0 [M + H]⁺ |
| | (R)-4-(but-3-yn-2-yloxy) | | | |
| | benzamide | | | |

Intermediates 151-152 shown in Table 5 were prepared in an analogous manner to Intermediate 141, following Step 2 from Scheme 27, using the appropriate ester Intermediates 153-154.

TABLE 5

| Inter- | Intermediate Structure and | Ester | ¹H NMR (400 MHz, | Mass |
|---|---|---|---|---|
| mediate | Name | Intermediate | DMSO-d6) δ ppm | spec m/z |
| 151 | 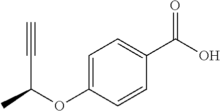 | 153 | 1.57 (d, J = 6.40 Hz, 3H), 3.57 (s, 1H), 5.18-5.23 (m, 1H), 7.07 (d, J = 8.77 Hz, 2H), 7.97 (d, J = 8.77 Hz, 2H), 12.65 (br s, 1H). | 191.0 [M + H]⁺ |
| | (S)-4-(but-3-yn-2- | | | |
| | yloxy)benzoic acid | | | |
| 152 | 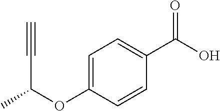 | 154 | 1.57 (d, J = 5.60 Hz, 3H), 3.57 (s, 1H), 5.17-5.22 (m, 1H), 7.06 (d, J = 7.80 Hz, 2H), 7.89 (d, J = 7.80 Hz, 2H), 12.65 (br s, 1H) | 191.0 [M + H]⁺ |
| | (R)-4-(but-3-yn-2- | | | |
| | yloxy)benzoic acid | | | |

### Intermediate 153: methyl (S)-4-(but-3-yn-2-yloxy)benzoate

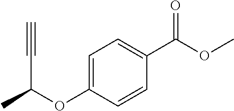

To solution of methyl 4-hydroxybenzoate (CAS No: 99-76-3, 10.0 g, 65.7 mmol), (R)-but-3-yn-2-ol (CAS No: 42969-65-3, 4.61 g, 65.7 mmol) and triphenylphosphine (17.2 g, 65.7 mmol) in tetrahydrofuran (100 mL) was added DIAD (13.3 g, 65.7 mmol) at 0° C. and the reaction mixture was stirred at room temperature for 16 h. The reaction mixture was poured into water (100 mL) and extracted with ethyl acetate (3×150 mL). The combined organic layers were separated, dried over anhydrous Na₂SO₄, filtered and concentrated in vacuo. The crude material was purified by combi-flash chromatography, by eluting with 30% ethyl acetate in heptane, to afford methyl (S)-4-(but-3-yn-2-yloxy)benzoate (Intermediate 153, 8.00 g, 60%) as a yellow solid. ¹H NMR (400 MHz, DMSO-d6) δ ppm 1.55 (d, J=6.80 Hz, 3H), 3.56 (s, 1H), 3.80 (s, 3H), 5.16-5.22 (m, 1H), 7.07 (d, J=8.60 Hz, 2H), 7.90 (d, J=8.60 Hz, 2H).

### Intermediates 154: methyl (R)-4-(but-3-yn-2-yloxy)benzoate

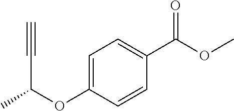

Intermediate 154 was synthesised in an analogous manner to Intermediate 153 using methyl 4-hydroxybenzoate (CAS No: 99-76-3) and (S)-but-3-yn-2-ol (CAS No: 2914-69-4) and exhibited: ¹H NMR (400 MHz, DMSO-d6) δ ppm 1.57 (d, J=6.40 Hz, 3H), 3.58 (d, J=2.0 Hz, 1H), 3.81 (s, 3H), 5.21-5.23 (m, 1H), 7.09 (d, J=8.60 Hz, 2H), 7.92 (d, J=8.60 Hz, 2H). Mass spec: m/z 205.0 [M+H]⁺.

### Example 26 was Synthesised Following Scheme 28

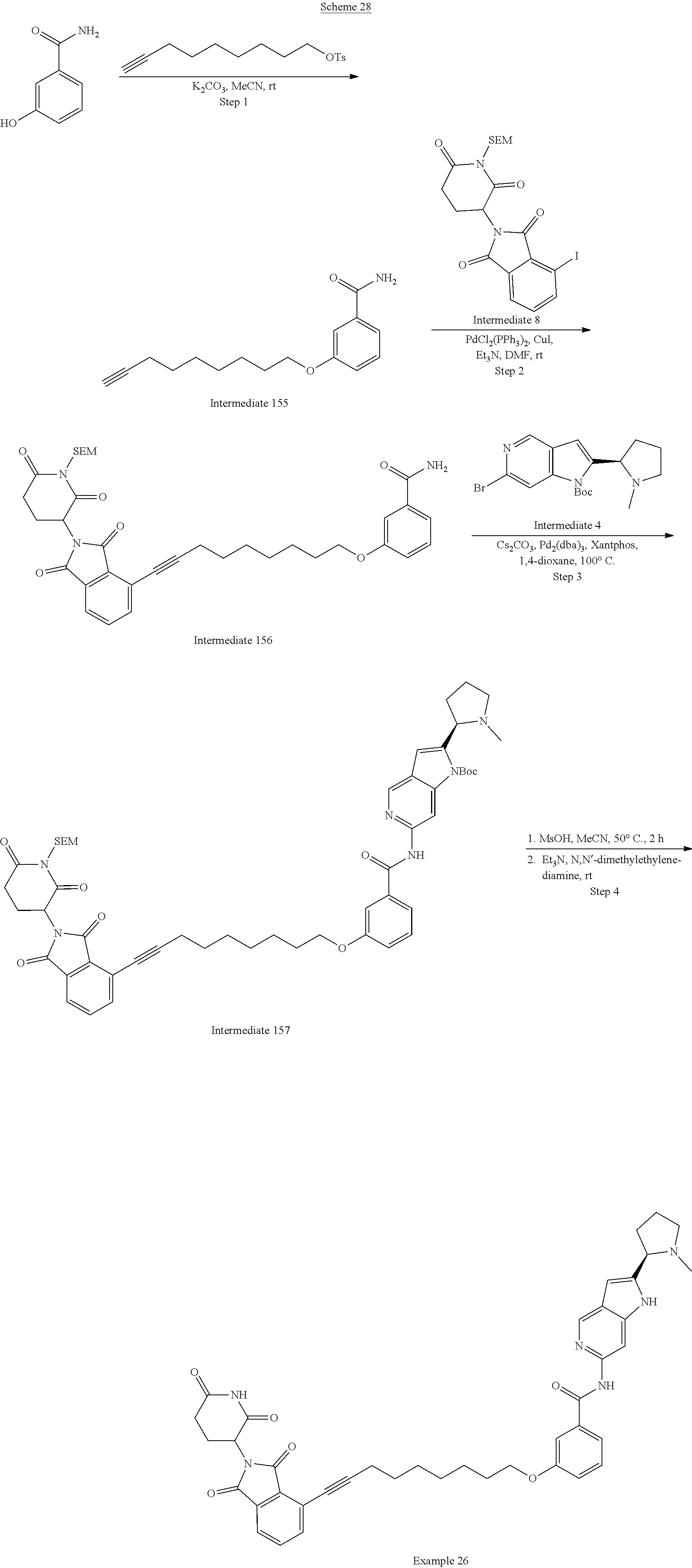

### Step 1

### Intermediate 155: 3-(non-8-yn-1-yloxy)benzamide

To solution of 3-hydroxybenzamide (CAS No: 618-49-5, 1.00 g, 7.29 mmol) in dimethylformamide (10 mL) was added potassium carbonate (1.53 g, 10.94 mmol) followed by non-8-yn-1-yl 4-methylbenzenesulfonate (CAS No: 87462-64-4, 2.15 g, 7.29 mmol) at room temperature and the reaction mixture was heated at 60° C. for 16 h. The reaction mixture was diluted with water (20 mL) and extracted with ethyl acetate (2×20 mL). The organic layer was separated, dried over anhydrous Na₂SO₄, filtered and concentrated in vacuo. The crude material was purified by combi-flash chromatography, by eluting with 60% ethyl acetate in heptane, to afford 3-(non-8-yn-1-yloxy)benzamide (Intermediate 155, 0.55 g, 29%) as an off white solid. ¹H NMR (400 MHz, DMSO-d₆) δ ppm 1.31-1.49 (m, 8H), 1.70-1.72 (m, 2H), 2.13-2.15 (m, 2H), 2.72 (s, 1H), 3.99-4.02 (m, 2H), 7.03-7.06 (m, 1H), 7.28-7.37 (m, 2H), 7.40-7.47 (m, 2H), 7.92 (br s, 1H). Mass spec: m/z: 260.0 [M+H]⁺.

### Step 2

### Intermediate 156: 3-((9-(2-(2,6-dioxo-1-((2-(trimethylsilyl)ethoxy)methyl)piperidin-3-yl)-1,3-dioxoisoindolin-4-yl)non-8-yn-1-yl)oxy)benzamide

To a solution of 3-(non-8-yn-1-yloxy)benzamide (Intermediate 155, 0.55 g, 3.86 mmol) in dimethylformamide (5 mL) was added 2-(2,6-dioxo-1-((2-(trimethylsilyl)ethoxy)methyl)piperidin-3-yl)-4-iodoisoindoline-1,3-dione (Intermediate 8, 1.0 g, 1.9 mmol) followed by triethylamine (19.4 mL, 139 mmol). The reaction mixture was purged with argon gas for 20 min. then copper(I) iodide (0.074 g, 0.38 mmol) and PdCl₂(PPh₃)₂ (0.27 g, 0.38 mmol) were added, and the reaction mixture was stirred at room temperature for 2 h. The reaction mixture was diluted with water (100 mL) and extracted with ethyl acetate (3×100 mL). The organic layer was separated, dried over anhydrous Na₂SO₄, filtered, and concentrated in vacuo. The crude material was purified by combi-flash chromatography, by eluting with 100% ethyl acetate in heptane, to afford 3-((9-(2-(2,6-dioxo-1-((2-(trimethylsilyl) ethoxy)methyl)piperidin-3-yl)-1,3-dioxoisoindolin-4-yl)non-8-yn-1-yl)oxy)benzamide (Intermediate 156, 1.3 g, 52%) as a yellow solid. ¹H NMR (400 MHz, DMSO-d₆) δ ppm −0.02 (s, 9H), 0.82-0.86 (m, 2H), 1.40-1.50 (m, 6H), 1.59-1.61 (m, 2H), 1.72-1.74 (m, 2H), 2.07-2.09 (m, 1H), 2.52-2.57 (m, 2H), 2.76-2.81 (m, 1H), 3.29-3.32 (m, 2H), 3.48-3.58 (m, 2H), 3.98-4.01 (m, 2H), 5.06-5.09 (m, 2H), 5.23-5.29 (m, 1H), 7.02-7.07 (m, 1H), 7.27-7.42 (m, 3H), 7.44 (br s, 1H), 7.78-7.87 (m, 3H), 7.91 (br s, 1H). Mass spec: m/z: 644.0 [M−H]⁻.

### Step 3

### Intermediate 157: tert-butyl 6-(3-((9-(2-(2,6-dioxo-1-((2-(trimethylsilyl)ethoxy)methyl)piperidin-3-yl)-1,3-dioxoisoindolin-4-yl)non-8-yn-1-yl)oxy)benzamido)-2-((R)-1-methylpyrrolidin-2-yl)-1H-pyrrolo[3,2-c]pyridine-1-carboxylate

To a solution of 3-((9-(2-(2,6-dioxo-1-((2-(trimethylsilyl)ethoxy)methyl)piperidin-3-yl)-1,3-dioxoisoindolin-4-yl)non-8-yn-1-yl)oxy)benzamide (Intermediate 156, 0.50 g, 0.77 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 0.29 g, 0.77 mmol) and cesium carbonate (0.51 g, 1.55 mmol. The reaction mixture was purged with argon gas for 10 min then Pd₂(dba)₃ (0.11 g, 0.12 mmol) and Xantphos (0.14 g, 0.23 mmol) were added at room temperature. The reaction mixture was further purged with argon gas for 15 min then heated at 100° C. for 2 h. The reaction mixture was then concentrated in vacuo and the residue was purified by combi-flash chromatography, by eluting with 80% ethyl acetate in heptane, to afford tert-butyl 6-(3-((9-(2-(2,6-dioxo-1-((2-(trimethylsilyl)ethoxy)methyl)piperidin-3-yl)-1,3-dioxoisoindolin-4-yl)non-8-yn-1-yl)oxy)benzamido)-2-((R)-1-methylpyrrolidin-2-yl)-1H-pyrrolo[3,2-c]pyridine-1-carboxylate (Intermediate 157, 0.60 g, 82%) as a yellow solid. ¹H NMR (400 MHz, DMSO-d₆) δ ppm −0.06 (s, 9H), 0.77-0.83 (m, 2H), 1.38-1.49 (m, 6H), 1.58-1.63 (m, 4H), 1.67 (s, 9H), 1.70-1.77 (m, 4H), 2.02-2.10 (m, 1H), 2.29-2.31 (m, 1H), 2.33 (s, 3H), 2.35-2.39 (m, 1H), 2.51-2.56 (m, 2H), 2.74-2.78 (m, 1H), 2.95-3.04 (m, 1H), 3.09-3.12 (m, 1H), 3.45-3.52 (m, 2H), 3.87-3.92 (m, 1H), 4.01-4.05 (m, 2H), 5.05 (s, 2H), 5.22-5.26 (m, 1H), 6.73 (s, 1H), 7.08 (m, 1H), 7.34-7.38 (m, 1H), 7.57-7.62 (m, 2H), 7.78-7.85 (m, 3H), 8.57 (s, 1H), 8.89 (s, 1H), 10.65 (br s, 1H). Mass spec: m/z: 945.1 [M+H]⁺

### Step 4

### Example 26: 3-((9-(2-(2,6-dioxopiperidin-3-yl)-1,3-dioxoisoindolin-4-yl)non-8-yn-1-yl)oxy)-N-(2-((R)-1-methylpyrrolidin-2-yl)-1H-pyrrolo[3,2-c]pyridin-6-yl)benzamide

To a solution of tert-butyl 6-(3-((9-(2-(2,6-dioxo-1-((2-(trimethylsilyl)ethoxy)methyl)piperidin-3-yl)-1,3-dioxoisoindolin-4-yl)non-8-yn-1-yl)oxy)benzamido)-2-((R)-1-methylpyrrolidin-2-yl)-1H-pyrrolo[3,2-c]pyridine-1-carboxylate (Intermediate 157, 0.30 g, 0.32 mmol) in acetonitrile (10 mL) was added methanesulfonic acid (0.21 mL, 3.17 mmol) at room temperature and the reaction mixture was heated at 50° C. for 2 h. The reaction mixture was cooled to room temperature then N,N′-dimethylethylenediamine (0.19 mL, 1.58 mmol) followed by triethylamine (0.88 mL, 6.35 mmol) were added and the reactions stirred at room temperature for 3 h. The reaction mixture was then concentrated in vacuo and water (20.0 mL) was added. The resulting precipitate was filtered, dried and the crude compound was purified by preparative HPLC (Method A) to afford 3-((9-(2-(2,6-dioxopiperidin-3-yl)-1,3-dioxoisoindolin-4-yl)non-8-yn-1-yl)oxy)-N-(2-((R)-1-methylpyrrolidin-2-yl)-1H-pyrrolo[3,2-c]pyridin-6-yl)benzamide (Example 26, 0.085 g, 37%) as an off white solid. ¹H NMR (400 MHz, DMSO-d₆) δ ppm 1.39-1.56 (m, 6H), 1.58-1.67 (m, 2H), 1.73-1.94 (m, 5H), 2.01-2.09 (m, 1H), 2.12-2.15 (m, 1H), 2.16 (s, 3H), 2.22-2.30 (m, 1H), 2.52-2.58 (m, 4H), 2.60-2.66 (m, 1H), 2.82-2.92 (m, 1H), 3.11-3.14 (m, 1H), 4.03-4.07 (m, 2H), 5.10-5.15 (m, 1H), 6.39 (s, 1H), 7.08-7.11 (m, 1H), 7.36-7.38 (m, 1H), 7.57-7.61 (m, 2H), 7.79-7.86 (m, 3H), 8.18 (s, 1H), 8.50 (s, 1H), 10.45 (br s, 1H), 11.12 (br s, 1H), 11.37 (br s, 1H). Mass spec: m/z: 715.1 [M+H]⁺.

### Example 27 was Synthesised Following Scheme 29

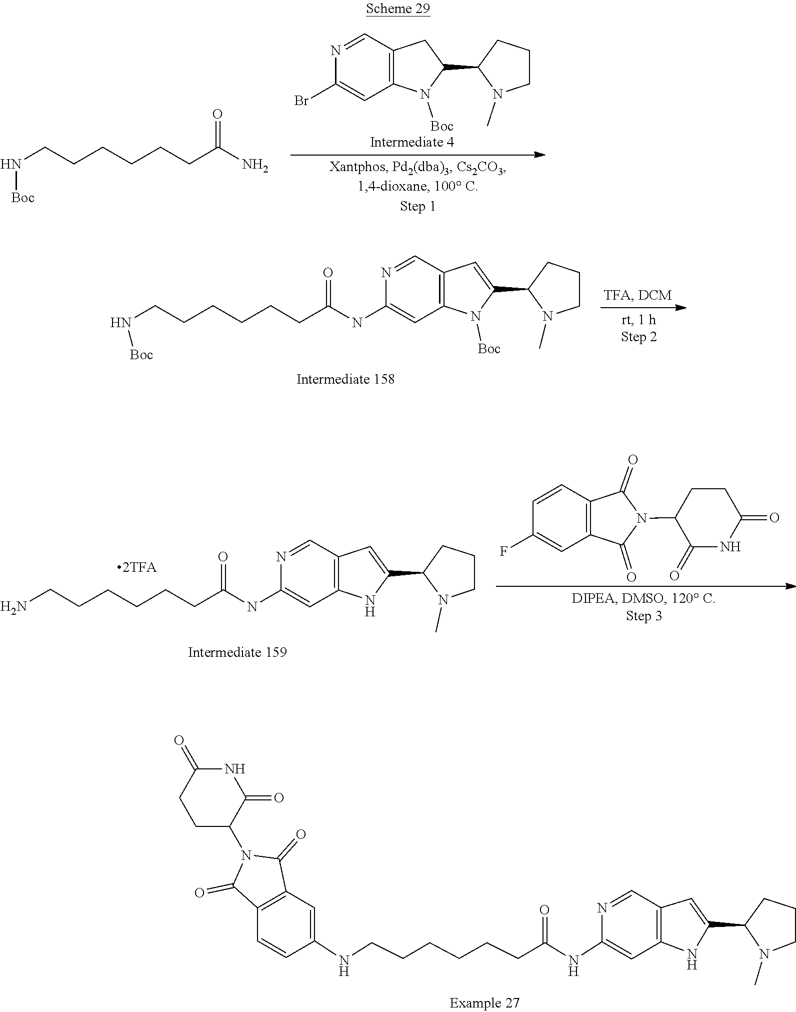

### Step 1

### Intermediate 158: tert-butyl 6-{7-[(tert-butoxycarbonyl)amino]heptanamido}-2-[(2R)-1-methylpyrrolidin-2-yl]pyrrolo[3,2-c]pyridine-1-carboxylate

A solution of tert-butyl N-(6-carbamoylhexyl)carbamate (CAS No: 51857-17-1, 0.3 g, 1.23 mmol), tert-butyl 6-bromo-2-[(2R)-1-methylpyrrolidin-2-yl]pyrrolo[3,2-c]pyridine-1-carboxylate (Intermediate 4, 0.42 g, 1.105 mmol), xantphos (0.28 g, 0.49 mmol), Pd₂(dba)₃ (0.22 g, 0.246 mmol) and Cs₂CO₃ (1.20 g, 3.684 mmol) in 1,4-dioxane (30 mL) was stirred for 4 h at 100° C., under N₂. The reaction was quenched with water (50 mL) and the aqueous phase was extracted with EtOAc (3×50 mL). The combined organic layers were washed with brine, dried over anhydrous Na₂SO₄, filtered and concentrated under reduced pressure. The residue was then chromatographed on a silica gel column, eluting with CH₂Cl₂/MeOH (94/6), to afford tert-butyl 6-{7-[(tert-butoxycarbonyl)amino]heptanamido}-2-[(2R)-1-methylpyrrolidin-2-yl]pyrrolo[3,2-c]pyridine-1-carboxylate (Intermediate 158, 0.6 g, 100%) as a yellow solid.

Mass spec: m/z: 544.7 [M+H]⁺.

### Step 2

### Intermediate 159: 7-amino-N-{2-[(2R)-1-methylpyrrolidin-2-yl]-1H-pyrrolo[3,2-c]pyridin-6-yl}heptanamide ditrifluoroacetate

A solution of tert-butyl 6-{7-[(tert-butoxycarbonyl)amino]heptanamido}-2-[(2R)-1-methylpyrrolidin-2-yl]pyrrolo[3,2-c]pyridine-1-carboxylate (Intermediate 158, 600 mg, 1.10 mmol) and TFA (4 mL) in DCM (8 mL) was stirred at room temperature for 2 h. The volatiles were then removed under reduced pressure to afford 7-amino —N-{2-[(2R)-1-methylpyrrolidin-2-yl]-1H-pyrrolo[3,2-c]pyridin-6-yl}heptanamide ditrifluoroacetate (Intermediate 159, 380 mg) as a yellow oil, which was used in the next step without further purification. Mass spec: m/z: 344.5 [M+H]⁺.

### Step 3

### Example 27: 7-{[2-(2,6-dioxopiperidin-3-yl)-1,3-dioxoisoindol-5-yl]amino}-N-{2-[(2R)-1-methylpyrrolidin-2-yl]-1H-pyrrolo[3,2-c]pyridin-6-yl}heptanamide

A solution of 7-amino-N-{2-[(2R)-1-methylpyrrolidin-2-yl]-1H-pyrrolo[3,2-c]pyridin-6-yl}heptanamide ditrifluoracetate (Intermediate 159, 200 mg, 0.58 mmol), 2-(2,6-dioxopiperidin-3-yl)-5-fluoroisoindole-1,3-dione (CAS No: 835616-61-0, 402 mg, 1.46 mmol) and DIPEA (753 mg, 5.820 mmol) in DMSO (5 mL) was stirred overnight at 120° C., under N₂. The resulting mixture was then purified directly by preparative HPLC (Column: YMC-Actus Triart C18ExRs, 30*150 mm, 5 μm; Mobile Phase A: Water (10 mmol/L NH₄HCO₃), Mobile Phase B: MeCN; Flow rate: 60 mL/min; Gradient: 25% B to 55% B in 10 min) to afford 7-{[2-(2,6-dioxopiperidin-3-yl)-1,3-dioxoisoindol-5-yl]amino}-N-{2-[(2R)-1-methylpyrrolidin-2-yl]-1H-pyrrolo[3,2-c]pyridin-6-yl}heptanamide (Example 27, 88.3 mg, 34%) as a yellow solid. ¹H NMR (400 MHz, DMSO-d6) δ ppm 11.25 (s, 1H), 11.05 (s, 1H), 10.11 (s, 1H), 8.40 (s, 1H), 8.08 (s, 1H), 7.55 (d, J=8.4 Hz, 1H), 7.10 (t, J=5.4 Hz 1H), 6.94 (s, 1H), 6.82-6.85 (m, 1H), 6.33 (s, 1H), 5.00-5.05 (m, 1H), 3.26-3.30 (m, 1H), 3.13-3.18 (m, 3H), 2.84-2.90 (m, 1H), 2.55-2.59 (m, 2H), 2.37 (t, J=7.4 Hz, 2H), 2.24-2.26 (m, 1H) 2.12-2.14 (m, 4H), 1.97-2.01 (m, 1H), 1.85-1.87 (m, 3H), 1.57-1.63 (m, 4H), 1.35-1.41 (m, 4H). Mass spec: m/z: 600.4 [M+H]⁺.

### Example 28 was Synthesised Following Scheme 30

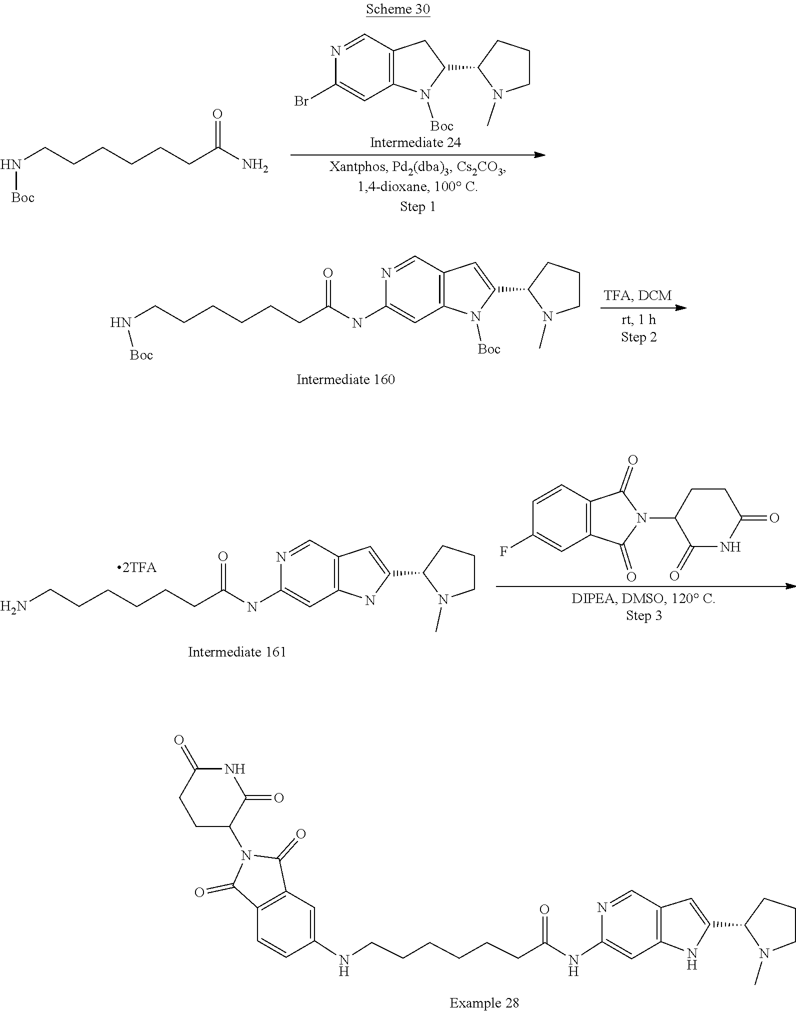

### Step 1

### Intermediate 160: tert-butyl 6-{7-[(tert-butoxycarbonyl)amino]heptanamido}-2-[(2S)-1-methylpyrrolidin-2-yl]pyrrolo[3,2-c]pyridine-1-carboxylate

Intermediate 160 was synthesised in an analogous manner to Intermediate 158, following Step 1 of Scheme 29, using tert-butyl N-(6-carbamoylhexyl)carbamate (CAS No: 51857-17-1) as the amide and tert-butyl 6-bromo-2-[(2S)-1-methylpyrrolidin-2-yl]pyrrolo[3,2-c]pyridine-1-carboxylate (Intermediate 24) as the aryl bromide, to afford tert-butyl 6-{7-[(tert-butoxycarbonyl)amino]heptanamido}-2-[(2S)-1-methylpyrrolidin-2-yl]pyrrolo[3,2-c]pyridine-1-carboxylate (Intermediate 160). Mass spec: m/z: 544.4 [M+H]⁺.

### Step 2

### Intermediate 161: 7-amino-N-{2-[(2S)-1-methylpyrrolidin-2-yl]-1H-pyrrolo[3,2-c]pyridin-6-yl}heptanamide ditrifluoracetate

Intermediate 161 was synthesised in an analogous manner to Intermediate 159, following Step 2 of Scheme 29, using tert-butyl 6-{7-[(tert-butoxycarbonyl)amino]heptanamido}-2-[(2S)-1-methylpyrrolidin-2-yl]pyrrolo[3,2-c]pyridine-1-carboxylate (Intermediate 160) as the amide, to afford 7-amino-N-{2-[(2S)-1-methylpyrrolidin-2-yl]-1H-pyrrolo[3,2-c]pyridin-6-yl}heptanamide ditrifluoracetate (Intermediate 161). Mass spec: m/z: 344.2 [M+H]⁺.

### Step 3

### Example 28: 7-{[2-(2,6-dioxopiperidin-3-yl)-1,3-dioxoisoindol-5-yl]amino}-N-{2-[(2S)-1-methylpyrrolidin-2-yl]-1H-pyrrolo[3,2-c]pyridin-6-yl}heptanamide

Example 28 was synthesised in an analogous manner to Example 27, following Step 3 of Scheme 29, using 7-amino-N-{2-[(2S)-1-methylpyrrolidin-2-yl]-1H-pyrrolo[3,2-c]pyridin-6-yl}heptanamide ditrifluoracetate (Intermediate 161) and 2-(2,6-dioxopiperidin-3-yl)-5-fluoroisoindole-1,3-dione (CAS No: 835616-61-0) as the aryl fluoride, to afford 7-{[2-(2,6-dioxopiperidin-3-yl)-1,3-dioxoisoindol-5-yl]amino}-N-{2-[(2S)-1-methylpyrrolidin-2-yl]-1H-pyrrolo[3,2-c]pyridin-6-yl}heptanamide (Example 28). ¹H NMR (400 MHz, DMSO-d6) δ ppm 11.25 (s, 1H), 11.05 (s, 1H), 10.11 (s, 1H), 8.40 (s, 1H), 8.08 (s, 1H), 7.55 (d, J=8.4 Hz, 1H), 7.10-7.12 (m, 1H), 7.09 (s, 1H), 6.84 (d, J=8.4 Hz, 1H), 6.33 (s, 1H), 5.00-5.05 (m, 1H), 3.28-3.33 (m, 2H), 3.13-3.16 (m, 3H), 2.91-2.92 (m, 1H), 2.86-2.88 (m, 1H), 2.35-2.41 (m, 2H), 2.35-2.39 (m, 1H), 2.14-2.20 (m, 4H), 1.90-1.99 (m, 1H), 1.84-1.89 (m, 3H), 1.65-1.71 (m, 4H), 1.45-1.55 (m, 4H). Mass spec: m/z: 600.4 [M+H]⁺.

### Example 29: 7-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}heptanamide

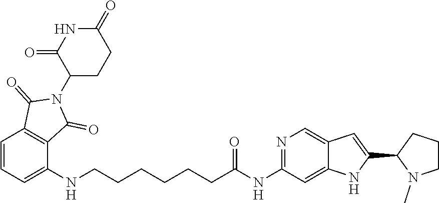 [SMILES: CN1CCC[C@@H]1c1cc2cnc(NC(=O)CCCCCCNc3cccc4c3C(=O)N(C3CCC(=O)NC3=O)C4=O)cc2[nH]1]

To a solution of 7-amino-N-{2-[(2R)-1-methylpyrrolidin-2-yl]-1H-pyrrolo[3,2-c]pyridin-6-yl}heptanamide ditrifluoroacetate (Intermediate 159, 190 mg, 0.498 mmol) in DMSO (4 mL) was added 2-(2,6-dioxopiperidin-3-yl)-4-fluoroisoindole-1,3-dione (CAS No: 835616-60-9, 275 mg, 0.996 mmol) and DIPEA (643 mg, 4.98 mmol) and the reaction mixture was stirred at 130° C. for 2 h. Water (2 mL) was added and the resulting mixture was chromatographed on a C18 silica column, eluting with MeCN/H₂O (+1-2 drops of formic acid) (24/76), to afford crude product. The crude product was then further purified by preparative HPLC (Column: XselectCSH Prep OBD C18 Column, 30*150 mm, 5 μm; Mobile Phase A: Water (+0.1% formic acid), Mobile Phase B: MeCN; Flow rate: 60 mL/min mL/min; Gradient: 2% B to 30% B in 8 min), to afford 7-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}heptanamide (Example 29, 108 mg, 36%) as a yellow solid. ¹H NMR (400 MHz, DMSO-d6) δ ppm 11.26 (s, 1H), 11.10 (s, 1H), 10.10 (s, 1H), 8.40 (s, 1H), 8.08 (s, 1H), 7.56 (t, J=8.0 Hz, 1H), 7.09 (d, J=8.8 Hz, 1H), 7.01 (d, J=6.8 Hz, 1H), 6.54 (t, J=6.0 Hz, 1H), 6.34 (s, 1H), 5.02-5.06 (m, 1H), 3.26-3.32 (m, 3H), 3.13-3.15 (m, 1H), 2.84-2.87 (m, 1H), 2.54-2.60 (m, 2H), 2.36 (t, J=7.2 Hz, 2H), 2.24-2.26 (m, 1H). 2.12-2.14 (m, 4H), 2.00-2.04 (m, 1H), 1.80-1.89 (m, 3H), 1.57-1.62 (m, 4H), 1.36 (m, 4H). Mass spec: m/z: 600.30 [M+H]⁺.

### Example 30: 9-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}nonanamide

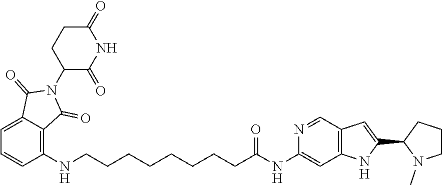 [SMILES: CN1CCC[C@@H]1c1cc2cnc(NC(=O)CCCCCCCCNc3cccc4c3C(=O)N(C3CCC(=O)NC3=O)C4=O)cc2[nH]1]

9-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}nonanamide (Example 30) was prepared in an analogous manner to Example 29, using 9-amino-N-{2-[(2R)-1-methylpyrrolidin-2-yl]-1H-pyrrolo[3,2-c]pyridin-6-yl}nonanamide ditrifluoroacetate (Intermediate 345) as the amine. Example 30 exhibited: ¹H NMR (400 MHz, MeOH-d4) δ ppm as 11.26 (s, 1H), 11.10 (s, 1H), 10.10 (s, 1H), 8.40 (s, 1H), 8.08 (s, 1H), 7.57 (t, J=7.6 Hz, 1H), 7.09 (d, J=8.8 Hz, 1H), 7.02 (d, J=6.8 Hz, 1H), 6.52-6.53 (m, 1H), 6.34 (s, 1H), 5.03-5.08 (m, 1H), 3.20-3.30 (m, 3H), 3.11-3.15 (m, 1H), 2.84-2.93 (m, 1H), 2.61-2.68 (m, 2H), 2.33-2.37 (m, 2H), 2.24-2.26 (m, 1H), 2.10-2.15 (m, 4H), 2.02-2.05 (m, 1H), 1.75-1.98 (m, 3H), 1.55-1.65 (m, 4H), 1.20-1.40 (m, 8H). Mass spec: m/z: 628.4 [M+H]⁺.

### Example 31 was Synthesised Following Scheme 31

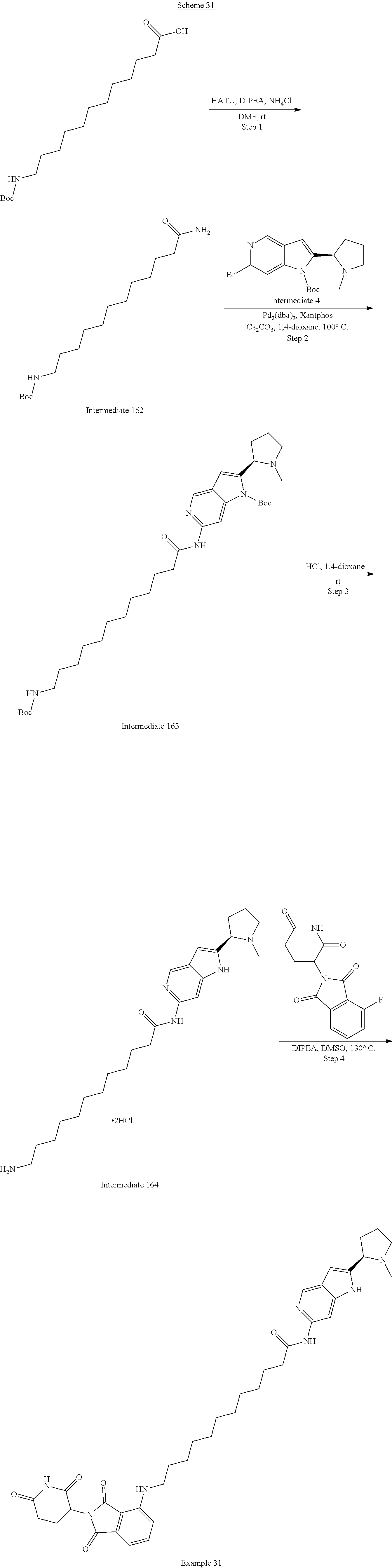

### Step 1

### Intermediate 162: tert-butyl N-(11-carbamoylundecyl)carbamate

To a solution of 12-[(tert-butoxycarbonyl)amino]dodecanoic acid (CAS No: 18934-81-1, 500 mg, 1.57 mmol), HATU (895 mg, 2.35 mmol), DIPEA (608 mg, 4.71 mmol) in anhydrous DMF (10 mL) was added NH₄Cl (420 mg, 7.84 mmol) and the reaction was stirred at room temperature for 2 h. The resulting mixture was chromatographed on a C18 silica column, eluting with MeCN/H₂O (with 1-2 drops formic acid) (45/55), to afford tert-butyl N-(11-carbamoylundecyl)carbamate (Intermediate 162, 460 mg, 84%) as a white solid. Mass spec: m/z 315.45 [M+H]⁺.

### Step 2

### Intermediate 163: tert-butyl 6-{12-[(tert-butoxycarbonyl)amino]dodecanamido}-2-[(2R)-1-methylpyrrolidin-2-yl]pyrrolo[3,2-c]pyridine-1-carboxylate

To a solution of tert-butyl N-(11-carbamoylundecyl)carbamate (Intermediate 162, 200 mg, 0.572 mmol) and tert-butyl 6-bromo-2-[(2R)-1-methylpyrrolidin-2-yl]pyrrolo[3,2-c]pyridine-1-carboxylate (Intermediate 4, 218 mg, 0.572 mmol) in 1,4-dioxane (4 mL) was added Cs₂CO₃ (373 mg, 1.144 mmol), XantPhos (66.24 mg, 0.114 mmol) and Pd₂(dba)₃ (52.42 mg, 0.057 mmol) under nitrogen and the mixture was stirred at 100° C. for 3 h. Water (5 mL) was added and the resulting solution was chromatographed on a C18 slicia column, eluting with MeCN/H₂O (with 1-2 drops formic acid) (52/48) to afford tert-butyl 6-{12-[(tert-butoxycarbonyl)amino]dodecanamido}-2-[(2R)-1-methylpyrrolidin-2-yl]pyrrolo[3,2-c]pyridine-1-carboxylate (Intermediate 163, 280 mg, 76%) as a yellow solid. Mass spec: m/z: 614.45 [M+H]⁺.

### Step 3

### Intermediate 164: 12-amino-N-{2-[(2R)-1-methylpyrrolidin-2-yl]-1H-pyrrolo[3,2-c]pyridin-6-yl}dodecanamide dihydrochloride

To a solution of tert-butyl 6-{12-[(tert-butoxycarbonyl)amino]dodecanamido}-2-[(2R)-1-methylpyrrolidin-2-yl]pyrrolo[3,2-c]pyridine-1-carboxylate (Intermediate 163, 280 mg, 0.433 mmol) in 1,4-dioxane (10 mL) was added HCl (12M, 4 mL) dropwise at room temperature and the reaction mixture was stirred at room temperature for 3 h. The volatiles were then removed under reduced pressure to afford 12-amino-N-{2-[(2R)-1-methylpyrrolidin-2-yl]-1H-pyrrolo[3,2-c]pyridin-6-yl}dodecanamide dihydrochloride (Intermediate 164, 180 mg) as a yellow solid, which was used in the next step without further purification. Mass spec: m/z: 414.20 [M+H]⁺.

### Step 4

### Example 31: 12-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}dodecanamide

To a solution of 12-amino-N-{2-[(2R)-1-methylpyrrolidin-2-yl]-1H-pyrrolo[3,2-c]pyridin-6-yl}dodecanamide dihydrochloride (Intermediate 164, 188 mg, 0.364 mmol) in DMSO (4 mL) was added 2-(2,6-dioxopiperidin-3-yl)-4-fluoroisoindole-1,3-dione (CAS No: 835616-60-9, 201 mg, 0.728 mmol) and DIPEA (470 mg, 3.640 mmol) and the reaction was stirred at 130° C. for 2 h. Water (2 mL) was added the resulting mixture was chromatographed on a C18 silica column, eluting with MeCN/H₂O (with 1-2 drops formic acid) (45/55), to give crude product. The crude product was further purified by preparative HPLC (Column: Xselect CSH Prep C18 Column, 30*150 mm, 5 μm; Mobile Phase A: Water (+0.1% formic acid), Mobile Phase B: MeCN; Flow rate: 60 mL/min; Gradient: 12% B to 42% B in 10 min) to afford 12-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}dodecanamide (Example 31, 72.6 mg, 29%) as a yellow solid. ¹H NMR (400 MHz, DMSO-d6) δ ppm 11.25 (s, 1H), 11.09 (s, 1H), 10.09 (s, 1H), 8.40 (s, 1H), 8.07 (s, 1H), 7.57 (t, J=8.0 Hz, 1H), 7.08 (d, J=8.8 Hz, 1H), 7.02 (d, J=6.8 Hz, 1H), 6.52 (t, J=6.0 Hz, 1H), 6.33 (s, 1H), 5.02-5.07 (m, 1H), 3.26-3.32 (m, 3H), 3.12 (t, J=7.6 Hz, 1H), 2.85-2.88 (m, 1H), 2.54-2.60 (m, 2H), 2.34 (t, J=7.2 Hz, 2H), 2.22-2.27 (m, 1H). 2.11-2.14 (m, 4H), 2.01-2.04 (m, 1H), 1.76-1.90 (m, 3H), 1.54-1.57 (m, 4H), 1.26 (br, 14H). Mass spec: m/z: 670.40 [M+H]⁺.

### Example 32 was Synthesised Following Scheme 32

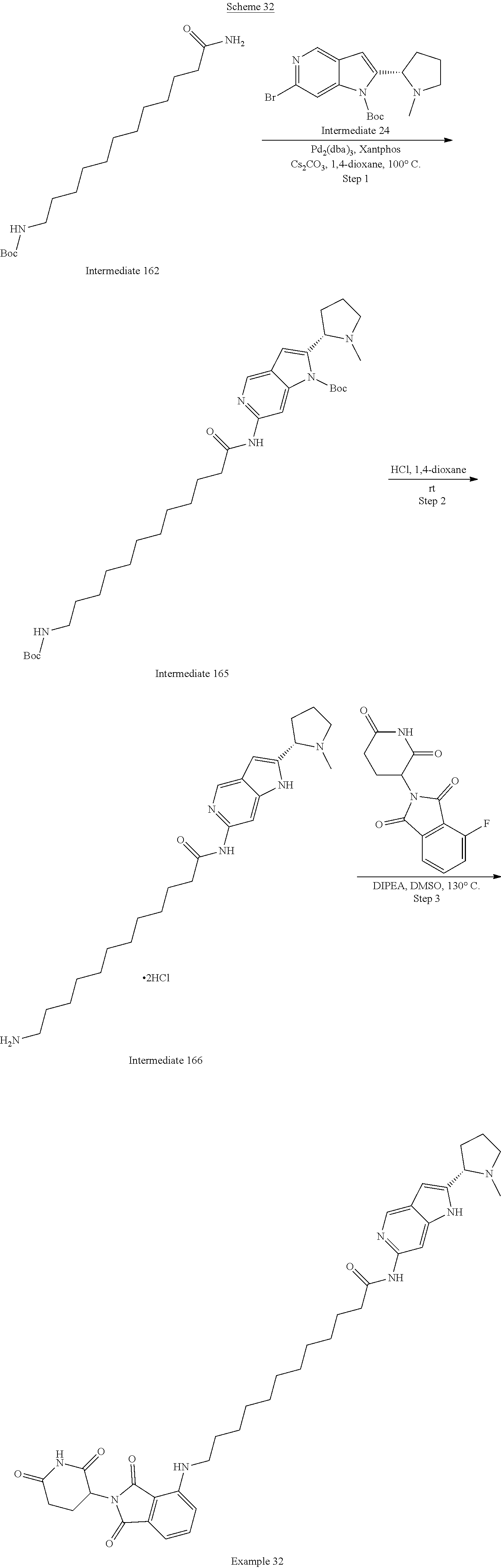 [SMILES: CC(C)(C)OC(=O)NCCCCCCCCCCCC(N)=O ; CN1CCC[C@H]1c1cc2cnc(Br)cc2n1C(=O)OC(C)(C)C ; CN1CCC[C@H]1c1cc2cnc(NC(=O)CCCCCCCCCCCNC(=O)OC(C)(C)C)cc2n1C(=O)OC(C)(C)C ; CN1CCC[C@H]1c1cc2cnc(NC(=O)CCCCCCCCCCCN)cc2[nH]1 ; O=C1CCC(N2C(=O)c3cccc(F)c3C2=O)C(=O)N1 ; CN1CCC[C@H]1c1cc2cnc(NC(=O)CCCCCCCCCCCNc3cccc4c3C(=O)N(C3CCC(=O)NC3=O)C4=O)cc2[nH]1]

### Step 1

### Intermediate 165: tert-butyl N-[11-({2-[(2S)-1-methylpyrrolidin-2-yl]-1H-pyrrolo[3,2-c]pyridin-6-yl}carbamoyl)undecyl]carbamate

Intermediate 165 was synthesised in an analogous manner to Intermediate 163, by following Step 2 shown in Scheme 31, using tert-butyl N-(11-carbamoylundecyl)carbamate (Intermediate 162) as the amide and tert-butyl 6-bromo-2-[(2S)-1-methylpyrrolidin-2-yl]pyrrolo[3,2-c]pyridine-1-carboxylate (Intermediate 24) as the aryl bromide, to afford tert-butyl N-[11-({2-[(2S)-1-methylpyrrolidin-2-yl]-1H-pyrrolo[3,2-c]pyridin-6-yl}carbamoyl)undecyl]carbamate (Intermediate 165). Mass spec: m/z: 514.40 [M+H]⁺.

### Step 2

### Intermediate 166: 12-amino-N-{2-[(2S)-1-methylpyrrolidin-2-yl]-1H-pyrrolo[3,2-c]pyridin-6-yl}dodecanamide dihydrochloride

Intermediate 166 was synthesised in an analogous manner to Intermediate 164, by following Step 3 shown in Scheme 31, using tert-butyl N-[11-({2-[(2S)-1-methylpyrrolidin-2-yl]-1H-pyrrolo[3,2-c]pyridin-6-yl}carbamoyl)undecyl]carbamate (Intermediate 165) as the amide, to afford 12-amino-N-{2-[(2S)-1-methylpyrrolidin-2-yl]-1H-pyrrolo[3,2-c]pyridin-6-yl}dodecanamide dihydrochloride (Intermediate 166). Mass spec: m/z: 414.50 [M+H]⁺.

### Step 3

### Example 32: 12-{[2-(2,6-dioxopiperidin-3-yl)-1,3-dioxoisoindol-4-yl]amino}-N-{2-[(2S)-1-methylpyrrolidin-2-yl]-1H-pyrrolo[3,2-c]pyridin-6-yl}dodecanamide

Example 32 was synthesised from Intermediate 166 in an analogous manner to Example 31, by following Step 4 shown in Scheme 31, using the following preparative HPLC conditions for the purification (Column: XBridge Prep OBD C18 Column, 30*150 mm, 5 μm; Mobile Phase A: Water (10 mmol/L NH₄HCO₃), Mobile Phase B: MeCN; Flow rate: 60 mL/min; Gradient: 50% B to 80% B in 7 min), to afford 12-{[2-(2,6-dioxopiperidin-3-yl)-1,3-dioxoisoindol-4-yl]amino}-N-{2-[(2S)-1-methylpyrrolidin-2-yl]-1H-pyrrolo[3,2-c]pyridin-6-yl}dodecanamide (Example 32). ¹H NMR (400 MHz, MeOH-d4) δ ppm 8.45 (s, 1H), 8.05 (s, 1H), 7.55 (t, J=8.0 Hz, 1H), 7.02-7.04 (m, 2H), 6.47 (s, 1H), 5.05-5.09 (m, 1H), 3.39-3.43 (m, 1H), 3.29-3.32 (m, 1H), 3.20-3.24 (m, 1H), 2.82-2.87 (m, 1H), 2.71-2.77 (m, 2H), 2.38-2.46 (m, 3H), 2.24-2.36 (m, 4H), 2.10-2.13 (m, 1H), 1.99-2.01 (m, 2H), 1.90-1.92 (m, 1H), 1.62-1.74 (m, 4H), 1.33-1.38 (m, 15H). Mass spec: m/z: 670.60 [M+H]⁺.

### Example 33: N-{3-chloro-2-[(2R)-1-methylpyrrolidin-2-yl]-1H-pyrrolo[3,2-c]pyridin-6-yl}-7-{[2-(2,6-dioxopiperidin-3-yl)-1,3-dioxoisoindol-4-yl]amino}heptanamide

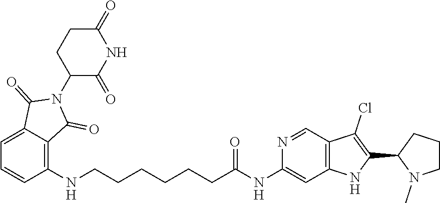 [SMILES: CN1CCC[C@@H]1c1[nH]c2cc(NC(=O)CCCCCCNc3cccc4c3C(=O)N(C3CCC(=O)NC3=O)C4=O)ncc2c1Cl]

To a solution of 7-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}heptanamide (Example 29, 50 mg, 0.083 mmol) in DMF (2.5 mL) was added NCS (14.5 mg, 0.108 mmol) and the reaction was stirred overnight at rt. The reaction was quenched by the addition of sat. aq. NH₄Cl (2 mL) and filtered. The resulting crude was then purified by preparative HPLC (Column: XBridge Prep OBD C18 Column, 30*150 mm, 5 μm; Mobile Phase A: Water (10 mmol/L NH₄HCO₃), Mobile Phase B: MeCN; Flow rate: 60 mL/min mL/min; Gradient: 40% B to 70% B in 7 min) to afford N-{3-chloro-2-[(2R)-1-methylpyrrolidin-2-yl]-1H-pyrrolo[3,2-c]pyridin-6-yl}-7-{[2-(2,6-dioxopiperidin-3-yl)-1,3-dioxoisoindol-4-yl]amino}heptanamide (Example 33, 15.1 mg, 28%) as a yellow solid. ¹H NMR (400 MHz, MeOH-d4) δ ppm 11.58 (s, 1H), 11.10 (s, 1H), 10.27 (s, 1H), 8.41 (s, 1H), 8.14 (s, 1H), 7.55-7.59 (m, 1H), 7.09 (d, J=8.8 Hz, 1H), 7.02 (d, J=6.4 Hz, 1H), 6.54 (s, 1H), 5.04-5.06 (m, 1H), 3.50-3.60 (m, 2H), 3.10-3.16 (m, 1H), 2.85-2.88 (m, 1H), 2.57-2.61 (m, 3H), 2.36-2.40 (m, 2H), 2.28-2.30 (m, 1H), 2.14-2.15 (m, 4H), 1.83-2.05 (m, 4H), 1.59-1.61 (m, 4H), 1.35-1.39 (m, 4H). Mass spec: m/z: 634.5 [M+H]⁺.

### Example 34 was Synthesised Following Scheme 33

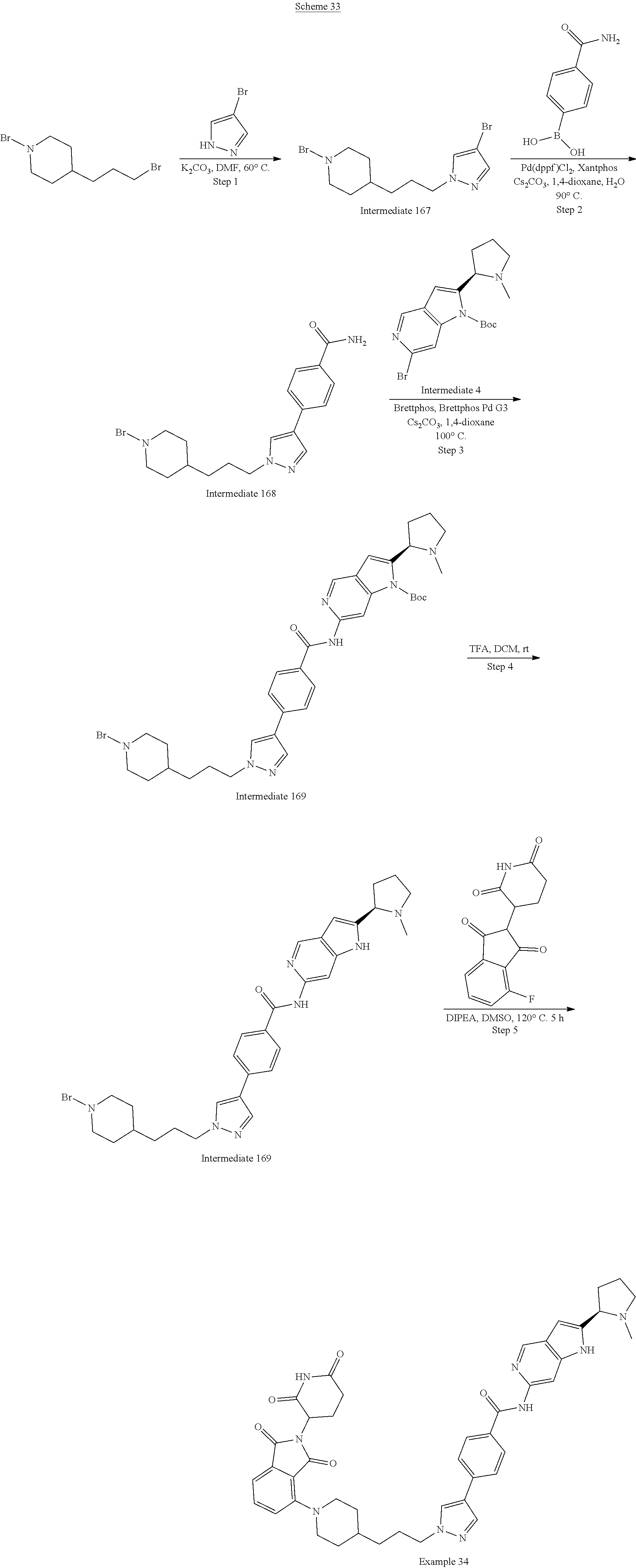

### Step 1

### Intermediate 167: tert-butyl 4-[3-(4-bromopyrazol-1-yl)propyl]piperidine-1-carboxylate

To a solution of tert-butyl 4-(3-bromopropyl)piperidine-1-carboxylate (CAS No: 164149-27-3, 4.9 g, 16.0 mmol) and 4-bromopyrazole (CAS No: 2075-45-8, 2.35 g, 16.0 mmol) in DMF (100 mL) was added K₂CO₃ (6.63 g, 48.0 mmol) and the reaction was stirred at 60° C. overnight. Water (100 mL) was added and the reaction mixture was extracted with EtOAc (3×200 mL). The combined organic layers was washed with brine (4×150 mL), dried over anhydrous sodium sulfate and concentrated under reduced pressure to afford tert-butyl 4-[3-(4-bromopyrazol-1-yl)propyl]piperidine-1-carboxylate (Intermediate 167, 6.00 g, 91%) as a colorless oil, which was used in the next step with out further purification. Mass spec: m/z: 372.1 [M+H]⁺.

### Step 2

### Intermediate 168: tert-butyl 4-{3-[4-(4-carbamoylphenyl)pyrazol-1-yl]propyl}piperidine-1-carboxylate

To a solution of tert-butyl 4-[3-(4-bromopyrazol-1-yl)propyl]piperidine-1-carboxylate (Intermediate 167, 3.00 g, 7.25 mmol) and 4-carbamoylphenylboronic acid (CAS No: 123088-59-5, 1.33 g, 8.06 mmol) in dioxane (168 mL) and H₂O (42 mL) was added Pd(dppf)Cl₂ (1.77 g, 2.42 mmol) and Cs₂CO₃ (3.94 g, 12.1 mmol) and the reaction was stirred at 90° C. for 4 h under N₂. Water (200 mL) was then added and the mixture was extracted with EtOAc (3×150 mL). The combined organic phases were washed with brine (200 mL), dried over anhydrous sodium sulfate and concentrated under reduced pressure. The resulting residue was purified on a silica gel column, eluting with DCM/MeOH (97/3) to afford tert-butyl 4-{3-[4-(4-carbamoylphenyl)pyrazol-1-yl]propyl}piperidine-1-carboxylate (Intermediate 168, 900 mg, 29%) as a brown solid. Mass spec: m/z: 313.15 [M-Boc+H]⁺.

### Step 3

### Intermediate 169: tert-butyl 4-{3-[4-(4-{[1-(tert-butoxycarbonyl)-2-[(2R)-1-methylpyrrolidin-2-yl]pyrrolo[3,2-c]pyridin-6-yl]carbamoyl}phenyl)pyrazol-1-yl]propyl}piperidine-1-carboxylate

To a solution of tert-butyl 4-{3-[4-(4-carbamoylphenyl)pyrazol-1-yl]propyl}piperidine-1-carboxylate (Intermediate 168, 250 mg, 0.606 mmol) and tert-butyl 6-bromo-2-[(2R)-1-methylpyrrolidin-2-yl]pyrrolo[3,2-c]pyridine-1-carboxylate (Intermediate 4, 276 mg, 0.727 mmol) in dioxane (10 mL) was added Brettphos (195 mg, 0.364 mmol), Brettphos Pd G3 (165 mg, 0.182 mmol), Cs₂CO₃ (494 mg, 1.52 mmol) and the reaction stirred overnight at 100° C. The reaction was cooled to room temperature and the residue chromatographed on a silica gel column, eluting with DCM/MeOH (95/5) to afford tert-butyl 4-{3-[4-(4-{[1-(tert-butoxycarbonyl)-2-[(2R)-1-methylpyrrolidin-2-yl]pyrrolo[3,2-c]pyridin-6-yl]carbamoyl}phenyl)pyrazol-1-yl]propyl}piperidine-1-carboxylate (Intermediate 169, 250 mg, 54%) as a brown solid. Mass spec: m/z: 712.6 [M+H]⁺.

### Step 4

### Intermediate 170: N-{2-[(2R)-1-methylpyrrolidin-2-yl]-1H-pyrrolo[3,2-c]pyridin-6-yl}-4-{1-[3-(piperidin-4-yl)propyl]pyrazol-4-yl}benzamide ditrifluoroacetate

To a solution of tert-butyl 4-{3-[4-(4-{[1-(tert-butoxycarbonyl)-2-[(2R)-1-methylpyrrolidin-2-yl]pyrrolo[3,2-c]pyridin-6-yl]carbamoyl}phenyl)pyrazol-1-yl]propyl}piperidine-1-carboxylate (Intermediate 169, 250 mg, 0.316 mmol) in DCM (5 mL) was added TFA (2.5 mL). The reaction was stirred at rt for 2 h and the volatiles were removed under reduced pressure to afford N-{2-[(2R)-1-methylpyrrolidin-2-yl]-1H-pyrrolo[3,2-c]pyridin-6-yl}-4-{1-[3-(piperidin-4-yl)propyl]pyrazol-4-yl}benzamide ditrifluoroacetate (Intermediate 170, 179 mg) as a brown oil, which was used in the next step without further purification. Mass spec: m/z: 512.4 [M+H]⁺.

### Step 5

### Example 34: 4-[1-(3-{1-[2-(2,6-dioxopiperidin-3-yl)-1,3-dioxoisoindol-4-yl]piperidin-4-yl}propyl)pyrazol-4-yl]-N-{2-[(2R)-1-methylpyrrolidin-2-yl]-1H-pyrrolo[3,2-c]pyridin-6-yl}benzamide

To a solution of N-{2-[(2R)-1-methylpyrrolidin-2-yl]-1H-pyrrolo[3,2-c]pyridin-6-yl}-4-{1-[3-(piperidin-4-yl)propyl]pyrazol-4-yl}benzamide ditrifluoroacetate (Intermediate 170, 40.0 mg, 0.07 mmol) and 2-(2,6-dioxopiperidin-3-yl)-4-fluoroisoindole-1,3-dione (CAS No: 835616-60-9, 32.4 mg, 0.117 mmol) in DMSO (2 mL) was added DIPEA (101 mg, 0.780 mmol) and the reaction was stirred at 120° C. for 5 h. The reaction mixture was then purified by preparative HPLC (Column: XBridge Prep OBD C18 Column, 30*150 mm, 5 μm; Mobile Phase A: Water (10 mmol/L NH₄HCO₃), Mobile Phase B: MeCN; Flow rate: 60 mL/min; Gradient: 38% B to 68% B in 13 min) to afford 4-[1-(3-{1-[2-(2,6-dioxopiperidin-3-yl)-1,3-dioxoisoindol-4-yl]piperidin-4-yl}propyl)pyrazol-4-yl]-N-{2-[(2R)-1-methylpyrrolidin-2-yl]-1H-pyrrolo[3,2-c]pyridin-6-yl}benzamide (Example 34, 20.0 mg, 35%) as a yellow solid. ¹H NMR (400 MHz, MeOH-d4) δ ppm 11.38 (s, 1H), 11.08 (s, 1H), 10.40 (s, 1H), 8.51 (s, 1H), 8.36 (s, 1H), 8.20 (s, 1H), 8.06 (d, J=8.4 Hz, 2H), 8.01 (s, 1H), 7.65-7.72 (m, 3H), 7.30-7.34 (m, 2H), 6.40 (s, 1H), 5.06-5.11 (m, 1H), 4.13-4.17 (m, 2H), 3.67-3.70 (m, 2H), 3.10-3.15 (m, 1H), 2.82-2.88 (m, 3H), 2.53-2.61 (m, 2H), 2.40-2.50 (m, 1H), 2.26-2.28 (m, 1H), 2.14-2.17 (m, 4H), 1.76-2.05 (m, 8H), 1.20-1.50 (m, 5H). Mass spec: m/z: 768.65 [M+H]⁺.

### Example 35: 4-[1-(3-{1-[2-(2,6-dioxopiperidin-3-yl)-1-oxo-3H-isoindol-4-yl]piperidin-4-yl}propyl)pyrazol-4-yl]-N-{2-[(2R)-1-methylpyrrolidin-2-yl]-1H-pyrrolo[3,2-c]pyridin-6-yl}benzamide

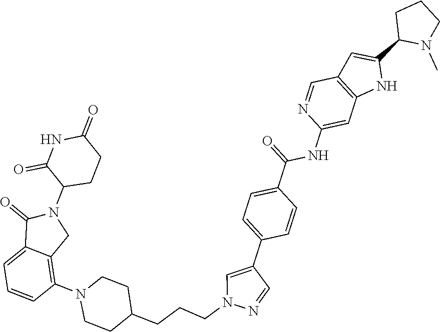 [SMILES: CN1CCC[C@@H]1c1cc2cnc(NC(=O)c3ccc(-c4cnn(CCCC5CCN(c6cccc7c6CN(C6CCC(=O)NC6=O)C7=O)CC5)c4)cc3)cc2[nH]1]

To a solution of N-{2-[(2R)-1-methylpyrrolidin-2-yl]-1H-pyrrolo[3,2-c]pyridin-6-yl}-4-{1-[3-(piperidin-4-yl)propyl]pyrazol-4-yl}benzamide (Intermediate 170, 100 mg, 0.195 mmol) and 3-(4-bromo-1-oxo-3H-isoindol-2-yl)piperidine-2,6-dione (CAS No: 2093387-36-9, 75.8 mg, 0.234 mmol) in 1,4-dioxane (5 mL) was added Cs₂CO₃ (191 mg, 0.585 mmol) and Pd-PEPPSI-IHept-Cl (CAS No:1814936-54-3, 24.6 mg, 0.0290 mmol) and the reaction was stirred overnight at 100° C., under N₂. The reaction mixture was then purified by preparative HPLC (Column: YMC-Actus Triart C18ExRs, 30*150 mm, 5 μm; Mobile Phase A: Water (with 10 mmol/L NH₄HCO₃), Mobile Phase B: MeCN; Flow rate: 60 mL/min; Gradient: 33% B to 63% B in 10 min) to afford 4-[1-(3-{1-[2-(2,6-dioxopiperidin-3-yl)-1-oxo-3H-isoindol-4-yl]piperidin-4-yl}propyl)pyrazol-4-yl]-N-{2-[(2R)-1-methylpyrrolidin-2-yl]-1H-pyrrolo[3,2-c]pyridin-6-yl}benzamide (Example 35, 11.7 mg, 7.7%) as a white solid. ¹H NMR (400 MHz, DMSO-d6) δ ppm as 11.38 (s, 1H), 11.00 (s, 1H), 10.41 (s, 1H), 8.51 (s, 1H), 8.36 (s, 1H), 8.21 (s, 1H), 8.06 (d, J=8.0 Hz, 2H), 8.02 (s, 1H), 7.72 (d, J=8.4 Hz, 2H), 7.40-7.42 (m, 1H), 7.29 (d, J=7.6 Hz, 1H), 7.16 (d, J=8.0 Hz, 1H), 6.40 (s, 1H), 5.05-5.15 (m, 1H), 4.40-4.44 (m, 1H), 4.26-4.30 (m, 1H), 4.14-4.17 (m, 2H), 3.35-3.40 (m, 2H), 3.10-3.20 (m, 1H), 2.85-2.95 (m, 1H), 2.65-2.75 (m, 2H), 2.55-2.60 (m, 2H), 2.45-2.50 (m, 1H), 2.20-2.30 (m, 1H), 2.10-2.20 (m, 4H), 1.90-1.99 (m, 1H), 1.79-1.90 (m, 4H), 1.77-1.79 (m, 3H), 1.38-1.50 (m, 1H), 1.20-1.38 (m, 4H). Mass spec: m/z: 754.70 [M+H]⁺.

### Example 36 was Synthesised Following Scheme 34

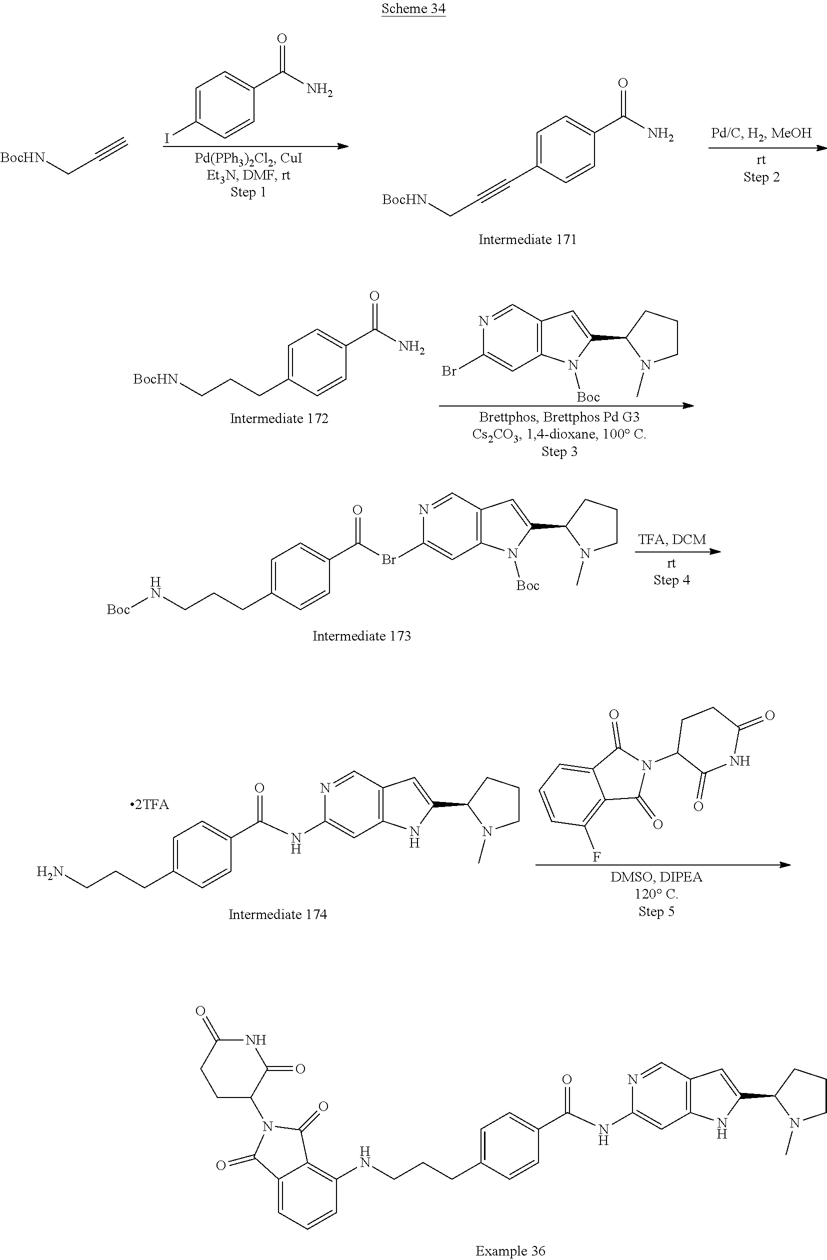

### Step 1

### Intermediate 171: tert-butyl N-[3-(4-carbamoylphenyl)prop-2-yn-1-yl]carbamate

To a solution of 4-iodobenzamide (CAS No: 3956-07-8, 800 mg, 3.23 mmol) in DMF (10 mL) was added tert-butyl N-(prop-2-yn-1-yl)carbamate (CAS No: 92136-39-5, 603 mg, 3.88 mmol), Pd(PPh₃)₂Cl₂ (227 mg, 0.32 mmol), CuI (61.6 mg, 0.324 mmol) and Et₃N (983 mg, 9.71 mmol) and the reaction was stirred at room temperature for 4 h under N₂. The mixture was quenched with water (20 mL) and the aqueous phase was extracted with EtOAc (3×20 mL). The combined organic layers were washed with brine, dried over anhydrous Na₂SO₄ and concentrated under reduced pressure. The resulting mixture was purified on a silica gel column, eluting with MeOH/DCM (6/94), to afford tert-butyl N-[3-(4-carbamoylphenyl)prop-2-yn-1-yl]carbamate (Intermediate 171, 800 mg, 90%) as a yellow solid. Mas spec: m/z: 275.0 [M+H]⁺.

### Step 2

### Intermediate 172: tert-butyl N-[3-(4-carbamoylphenyl)propyl]carbamate

To a solution of tert-butyl N-[3-(4-carbamoylphenyl)prop-2-yn-1-yl]carbamate (Intermediate 171, 600 mg, 2.18 mmol) in MeOH (10 mL) was added Pd/C (10% w/w, 300 mg, 2.81 mmol) and hydrogen (3 atm) was introduced and the reaction was stirred at room temperature for 2 h. The reaction was then filtered through a Celite pad washing with MeOH. The volatiles were then concentrated under reduced pressure to afford tert-butyl N-[3-(4-carbamoylphenyl)propyl]carbamate (Intermediate 172, 500 mg, 91%) as a yellow solid, which was used in the next step without further purification. Mass spec m/z: 279.0 [M+H]⁺.

### Step 3

### Intermediate 173: tert-butyl 6-(4-{3-[(tert-butoxycarbonyl)amino]propyl}benzamido)-2-[(2R)-1-methylpyrrolidin-2-yl]pyrrolo[3,2-c]pyridine-1-carboxylate

To a solution of tert-butyl N-[3-(4-carbamoylphenyl)propyl]carbamate (Intermediate 172, 250 mg, 0.9 mmol) and tert-butyl 6-bromo-2-[(2R)-1-methylpyrrolidin-2-yl]pyrrolo[3,2-c]pyridine-1-carboxylate (Intermediate 4, 342 mg, 0.9 mmol) in 1,4-dioxane (10 mL) was added Cs₂CO₃ (585.2 mg, 1.79 mmol), Brettphos (289.2 mg, 0.539 mmol) and Brettphos Pd G3 (244.2 mg, 0.269 mmol) and the reaction was stirred overnight at 100° C. under the N₂. The volatiles were removed under reduced pressure and the resulting residue was chromatographed on a silica gel column, eluting with MeOH/DCM (6/94), to afford tert-butyl 6-(4-{3-[(tert-butoxycarbonyl)amino]propyl}benzamido)-2-[(2R)-1-methylpyrrolidin-2-yl]pyrrolo[3,2-c]pyridine-1-carboxylate (Intermediate 173, 190 mg, 36%) as a yellow solid. Mass spec: m/z: 578.0 [M+H]⁺.

### Step 4

### Intermediate 174: 4-(3-aminopropyl)-N-{2-[(2R)-1-methylpyrrolidin-2-yl]-1H-pyrrolo[3,2-c]pyridin-6-yl}benzamide ditrifluoroacetate

To a solution of tert-butyl 6-(4-{3-[(tert-butoxycarbonyl)amino]propyl}benzamido)-2-[(2R)-1-methylpyrrolidin-2-yl]pyrrolo[3,2-c]pyridine-1-carboxylate (Intermediate 173, 190 mg, 0.329 mmol) in DCM (6 mL) was added TFA (2 mL). The reaction was stirred at room temperature for 2 h and concentrated under reduced pressure to get 4-(3-aminopropyl)-N-{2-[(2R)-1-methylpyrrolidin-2-yl]-1H-pyrrolo[3,2-c]pyridin-6-yl}benzamide ditrifluoroacetate (Intermediate 174, 120 mg, 97%) as a yellow solid, which was used in the next step without further purification. Mass spec: m/z: 378.0 [M+H]⁺.

### Step 5

### Example 36: 4-(3-{[2-(2,6-dioxopiperidin-3-yl)-1,3-dioxoisoindol-4-yl]amino}propyl)-N-{2-[(2R)-1-methylpyrrolidin-2-yl]-1H-pyrrolo[3,2-c]pyridin-6-yl}benzamide

To a solution of 4-(3-aminopropyl)-N-{2-[(2R)-1-methylpyrrolidin-2-yl]-1H-pyrrolo[3,2-c]pyridin-6-yl}benzamide ditrifluoroacetate (Intermediate 174, 120 mg, 0.32 mmol) and 2-(2,6-dioxopiperidin-3-yl)-4-fluoroisoindole-1,3-dione (CAS No: 835616-60-9, 87.8 mg, 0.318 mmol) in DMSO (4 mL) was added DIPEA (411 mg, 3.18 mmol) and the reaction was stirred at 120° C. for 4 h. The reaction mixture was cooled and and purified by preparative HPLC (Column: YMC-Actus Triart C18ExRs, 30*150 mm, 5 μm; Mobile Phase A: Water (10 mmol/L NH₄HCO₃), Mobile Phase B: MeCN; Flow rate: 60 mL/min; Gradient: 33% B to 63% B in 7 min) to afford 4-(3-{[2-(2,6-dioxopiperidin-3-yl)-1,3-dioxoisoindol-4-yl]amino}propyl)-N-{2-[(2R)-1-methylpyrrolidin-2-yl]-1H-pyrrolo[3,2-c]pyridin-6-yl}benzamide (Example 36, 34.1 mg, 17%) as a yellow solid. ¹H NMR (400 MHz, DMSO-d6) δ ppm 11.36 (s, 1H), 11.10 (s, 1H), 10.36 (s, 1H), 8.50 (s, 1H), 8.19 (s, 1H), 7.99 (d, J=8.0 Hz, 2H), 7.58 (t, J=8.0 Hz, 1H), 7.36 (d, J=8.4 Hz, 2H), 7.02-7.09 (m, 2H), 6.63 (t, J=6.4 Hz, 1H), 6.39 (s, 1H), 5.04-5.08 (m, 1H), 3.32-3.36 (m, 2H), 3.26-3.28 (m, 1H), 3.12-3.16 (m, 1H), 2.85-2.89 (m, 1H), 2.74-2.78 (m, 2H), 2.51-2.55 (m, 3H), 2.25-2.33 (m, 1H), 2.14-2.19 (m, 4H), 2.00-2.05 (m, 1H), 1.98-1.99 (m, 3H), 1.89-1.96 (m, 1H). Mass spec: m/z: 634.3 [M+H]⁺.

### Example 37 was Synthesised Following Scheme 35

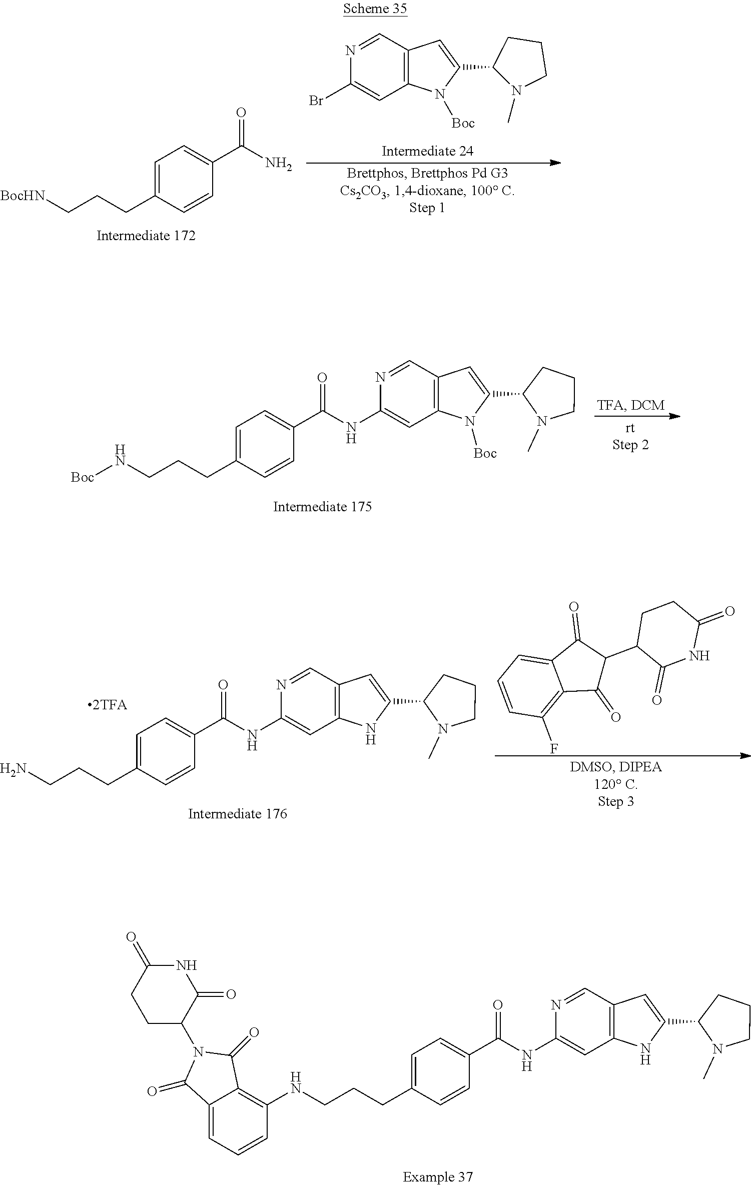

### Step 1

### Intermediate 175: tert-butyl 6-(4-{3-[(tert-butoxycarbonyl)amino]propyl}benzamido)-2-[(2S)-1-methylpyrrolidin-2-yl]pyrrolo[3,2-c]pyridine-1-carboxylate

Intermediate 175 was synthesised in an analogous manner to Intermediate 173 following Step 3 in Scheme 34, using tert-butyl N-[3-(4-carbamoylphenyl)propyl]carbamate (Intermediate 172) as the amide and tert-butyl 6-bromo-2-[(2S)-1-methylpyrrolidin-2-yl]pyrrolo[3,2-c]pyridine-1-carboxylate (Intermediate 24) as the aryl bromide, to afford tert-butyl 6-(4-{3-[(tert-butoxycarbonyl)amino]propyl}benzamido)-2-[(2S)-1-methylpyrrolidin-2-yl]pyrrolo[3,2-c]pyridine-1-carboxylate (Intermediate 175). Mass spec: m/z: 578.3 [M+H]⁺.

### Step 2

### Intermediate 176: 4-(3-aminopropyl)-N-{2-[(2S)-1-methylpyrrolidin-2-yl]-1H-pyrrolo[3,2-c]pyridin-6-yl}benzamide

Intermediate 176 was synthesised in an analogous manner to Intermediate 174 following Step 4 in Scheme 34, using tert-butyl 6-(4-{3-[(tert-butoxycarbonyl)amino]propyl}benzamido)-2-[(2S)-1-methylpyrrolidin-2-yl]pyrrolo[3,2-c]pyridine-1-carboxylate (Intermediate 175) as the amide, to afford 4-(3-aminopropyl)-N-{2-[(2S)-1-methylpyrrolidin-2-yl]-1H-pyrrolo[3,2-c]pyridin-6-yl}benzamide ditrifluoroacetate (Intermediate 176). Mass spec: m/z: 378.2 [M+H]⁺.

### Step 3

### Example 37: 4-(3-{[2-(2,6-dioxopiperidin-3-yl)-1,3-dioxoisoindol-4-yl]amino}propyl)-N-{2-[(2S)-1-methylpyrrolidin-2-yl]-1H-pyrrolo[3,2-c]pyridin-6-yl}benzamide

Example 37 was synthesised in an analogous manner to Example 36, following Step 5 in Scheme 34, using 4-(3-aminopropyl)-N-{2-[(2S)-1-methylpyrrolidin-2-yl]-1H-pyrrolo[3,2-c]pyridin-6-yl}benzamide ditrifluoroacetate (Intermediate 176) as the amine and 2-(2,6-dioxopiperidin-3-yl)-4-fluoroisoindole-1,3-dione (CAS No: 835616-60-9) as the aryl fluoride, to afford 4-(3-{[2-(2,6-dioxopiperidin-3-yl)-1,3-dioxoisoindol-4-yl]amino}propyl)-N-{2-[(2S)-1-methylpyrrolidin-2-yl]-1H-pyrrolo[3,2-c]pyridin-6-yl}benzamide (Example 37). ¹H NMR (400 MHz, MeOH-d4) δ ppm 8.55 (s, 1H), 8.19 (s, 1H), 7.92 (d, J=8.0 Hz, 2H), 7.55 (t, J=7.2 Hz, 1H), 7.41 (d, J=8.0 Hz, 2H), 7.00-7.06 (m, 2H), 6.52 (s, 1H), 5.03-5.08 (m, 1H), 3.40-3.47 (m, 3H), 3.24-3.26 (m, 1H), 2.80-2.89 (m, 3H), 2.70-2.74 (m, 2H), 2.40-2.42 (m, 1H), 2.29-2.33 (m, 4H), 2.02-2.13 (m, 5H), 1.92-1.94 (m, 1H). Mass spec: m/z: 634.5 [M+H]⁺.

### Example 38 was Synthesised Following Scheme 36

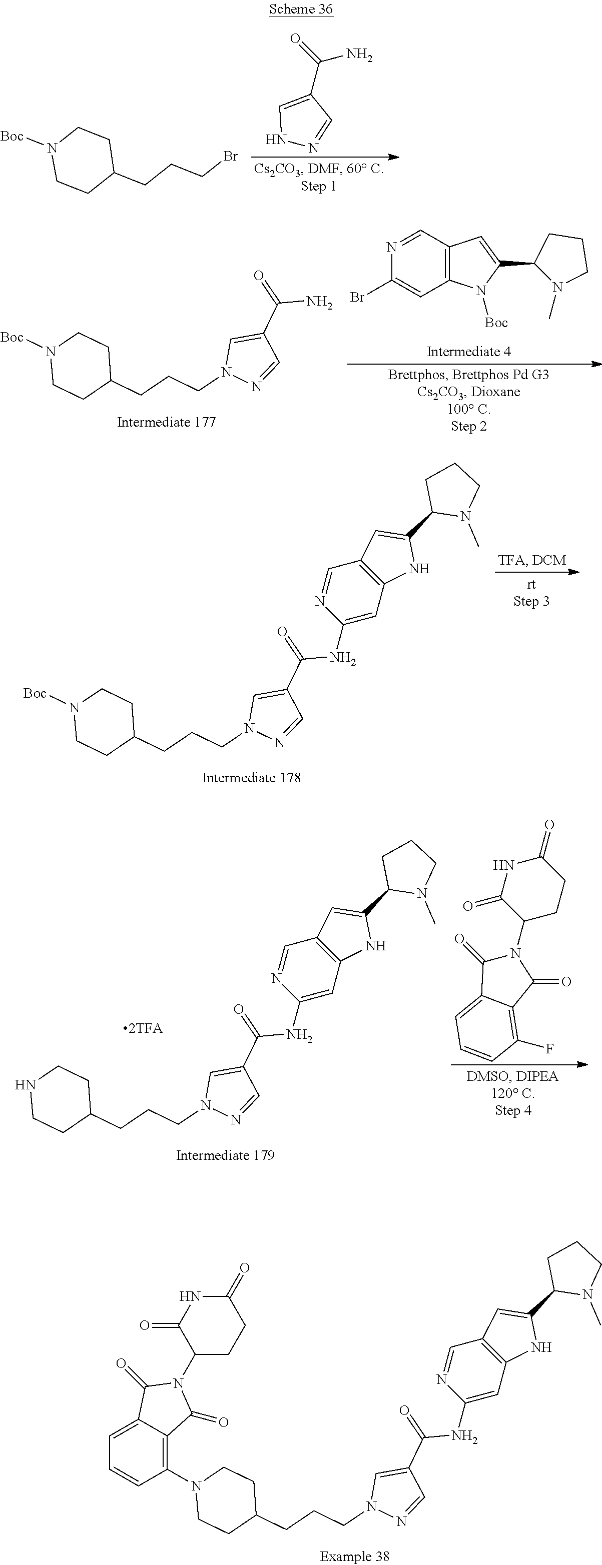

### Step 1

### Intermediate 177: tert-butyl 4-[3-(4-carbamoylpyrazol-1-yl)propyl]piperidine-1-carboxylate

To a solution of tert-butyl 4-(3-bromopropyl)piperidine-1-carboxylate (CAS No: 164149-27-3, 600 mg, 1.95 mmol) in DMF (20 mL) was added 1H-pyrazole-4-carboxamide (CAS No: 437701-80-9, 217 mg, 1.95 mmol) and Cs₂CO₃ (1915 mg, 5.87 mmol) and the reaction was stirred overnight at 60° C. for 2 h. The mixture was quenched with water (100 mL) and extracted with EtOAc (3×100 mL). The combined organic layers were washed with brine, dried over anhydrous Na₂SO₄ and the volatiles removed under reduced pressure. The resulting crude was chromatographed on a silical gel column with MeOH/DCM (5/95) to afford tert-butyl 4-[3-(4-carbamoylpyrazol-1-yl)propyl]piperidine-1-carboxylate (Intermediate 177, 650 mg, 91%) as a yellow solid. Mass spec: m/z: 337 [M+H]⁺.

### Step 2

### Intermediate 178: tert-butyl 4-{3-[4-({2-[(2R)-1-methylpyrrolidin-2-yl]-1H-pyrrolo[3,2-c]pyridin-6-yl}carbamoyl)pyrazol-1-yl]propyl}piperidine-1-carboxylate

To a solution of tert-butyl 4-[3-(4-carbamoylpyrazol-1-yl)propyl]piperidine-1-carboxylate (Intermediate 177, 200 mg, 0.594 mmol) in 1,4-dioxane (5 mL) was added tert-butyl 6-bromo-2-[(2R)-1-methylpyrrolidin-2-yl]pyrrolo[3,2-c]pyridine-1-carboxylate (Intermediate 4, 226 mg, 0.594 mmol), Brettphos (191 mg, 0.356 mmol), Brettphos Pd G3 (161 mg, 0.178 mmol) and Cs₂CO₃ (387 mg, 1.18 mmol) and the reaction was stirred overnight at 100° C. under N₂. The crude mixture was then purified on a silical gel column with MeOH/DCM (5/95) to afford tert-butyl 4-{3-[4-({2-[(2R)-1-methylpyrrolidin-2-yl]-1H-pyrrolo[3,2-c]pyridin-6-yl}carbamoyl)pyrazol-1-yl]propyl}piperidine-1-carboxylate (Intermediate 178, 300 mg, 82%) as a yellow solid. Mass spec: m/z: 536 [M+H]⁺.

### Step 3

### Intermediate 179: N-{2-[(2R)-1-methylpyrrolidin-2-yl]-1H-pyrrolo[3,2-c]pyridin-6-yl}-1-[3-(piperidin-4-yl)propyl]pyrazole-4-carboxamide ditrifluroacetate

To a solution of tert-butyl 4-{3-[4-({2-[(2R)-1-methylpyrrolidin-2-yl]-1H-pyrrolo[3,2-c]pyridin-6-yl}carbamoyl)pyrazol-1-yl]propyl}piperidine-1-carboxylate (Intermediate 178, 150 mg, 0.28 mmol) in DCM (2 mL) was added TFA (1 mL) and the reaction mixture was stirred at room temperature for 1 h. The volatiles were then removed under reduced pressure to afford N-{2-[(2R)-1-methylpyrrolidin-2-yl]-1H-pyrrolo[3,2-c]pyridin-6-yl}-1-[3-(piperidin-4-yl)propyl]pyrazole-4-carboxamide ditrifluroacetate (Intermediate 179, 122 mg) as a yellow oil, which was used in the next step without further purification. Mass spec: m/z: 436 [M+H]⁺.

### Step 4

### Example 38: 1-(3-{1-[2-(2,6-dioxopiperidin-3-yl)-1,3-dioxoisoindol-4-yl]piperidin-4-yl}propyl)-N-{2-[(2R)-1-methylpyrrolidin-2-yl]-1H-pyrrolo[3,2-c]pyridin-6-yl}pyrazole-4-carboxamide

To a solution of N-{2-[(2R)-1-methylpyrrolidin-2-yl]-1H-pyrrolo[3,2-c]pyridin-6-yl}-1-[3-(piperidin-4-yl)propyl]pyrazole-4-carboxamide ditrifluoroacetate (Intermediate 179, 100 mg, 0.23 mmol) in DMSO (2 mL) was added 2-(2,6-dioxopiperidin-3-yl)-4-fluoroisoindole-1,3-dione (CAS No: 835616-60-9, 126 mg, 0.460 mmol) and DIPEA (89.0 mg, 0.690 mmol) and the reaction was stirred at 120° C. overnight. The resulting crude product was then purified by preparative HPLC (Column: WelFlash C18, Regular C18 20-40 μm, 120 g; Mobile Phase A: Water (10 mmol/L NH₄HCO₃+0.05% NH₃·H₂O), Mobile Phase B: MeCN; Flow rate: 60 mL/min mL/min; Gradient: 30% B to 60% B in 30 min; Wave Length: 254 nm/220 nm nm) to afford 1-(3-{1-[2-(2,6-dioxopiperidin-3-yl)-1,3-dioxoisoindol-4-yl]piperidin-4-yl}propyl)-N-{2-[(2R)-1-methylpyrrolidin-2-yl]-1H-pyrrolo[3,2-c]pyridin-6-yl}pyrazole-4-carboxamide (Example 38, 38.0 mg, 23%) as a yellow solid. ¹H NMR (400 MHz, MeOH-d4) δ ppm 8.51 (s, 1H), 8.31 (s, 1H), 2.11-2.12 (m, 2H), 7.63-7.66 (m, 1H), 7.31-7.35 (m, 2H), 6.52 (s, 1H), 5.08-5.13 (m, 1H), 4.25-4.28 (m, 2H), 3.75-3.78 (m, 2H), 3.46-3.50 (m, 1H), 3.20-3.30 (m, 1H), 2.83-2.93 (m, 3H), 2.72-2.78 (m, 2H), 2.40-2.42 (m, 1H), 2.20-2.31 (m, 4H), 1.80-2.12 (m, 7H), 1.30-1.60 (m, 6H). Mass spec: m/z: 692.3 [M+H]⁺.

### Example 39 was Synthesised Following Scheme 37

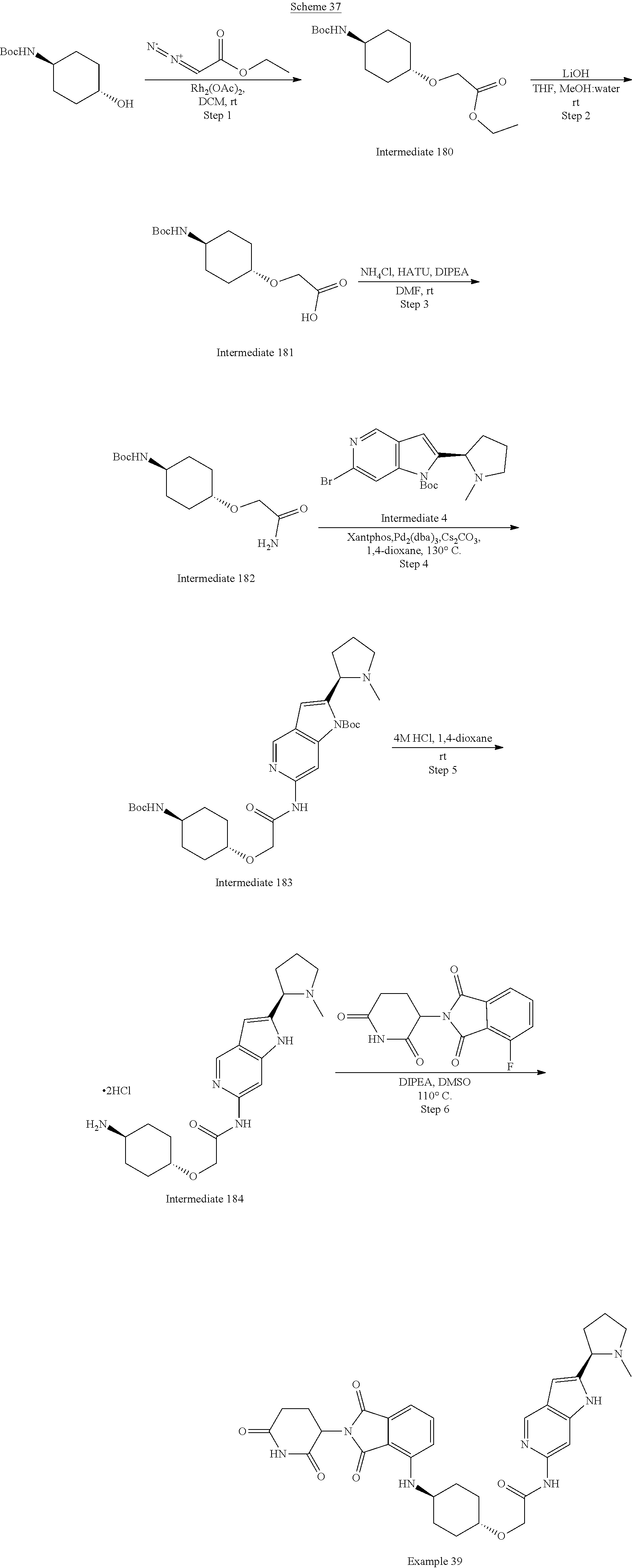

### Step 1

### Intermediate 180: Ethyl 2-(((1r, 4r)-4-((tert-butoxycarbonyl)amino)cyclohexyl)oxy)acetate

To a solution of tert-butyl ((1r,4r)-4-hydroxycyclohexyl)carbamate (CAS No: 111300-06-2, 10.0 g, 46.4 mmol) in dichloromethane (50 mL) was added rhodium(II) acetate dimer (1.04 g, 2.32 mmol) at room temperature and the reaction mixture was stirred at room temperature for 10 min. Ethyl 2-diazoacetate (6.36 g, 55.7 mmol) was then added and the reaction stirred for 16 h. The reaction mixture was poured into water (100 mL) and extracted with DCM (3×80 mL). The combined organic layers were dried over anhydrous Na₂SO₄ and concentrated in vacuo. The resulting residue was purified by combiflash column chromatography (20% EtOAc in heptane) to afford ethyl 2-(((1r, 4r)-4-((tert-butoxycarbonyl)amino)cyclohexyl)oxy)acetate (Intermediate 180, 8.0 g, 57%) as an off white solid. ¹H NMR (400 MHz, DMSO-d₆) δ ppm 1.11-1.17 (m, 3H), 1.18-1.21 (m, 4H), 1.37 (s, 9H), 1.73-1.77 (m, 2H), 1.91-1.98 (m, 2H), 3.16-3.27 (m, 2H), 4.07-4.13 (m, 2H), 4.19 (s, 2H), 6.68 (d, J=7.50 Hz, 1H).

### Step 2

### Intermediate 181: 2-(((1r, 4r)-4-((tert-butoxycarbonyl)amino)cyclohexyl)oxy)acetic acid

To a solution of ethyl 2-(((1r, 4r)-4-((tert-butoxycarbonyl)amino)cyclohexyl)oxy)acetate (Intermediate 180, 7.5 g, 25 mmol) in THF (40 mL), MeOH (20 mL) and water (30 mL) was added LiOH (1.8 g, 75 mmol) and the reaction was stirred at room temperature for 3 h. The reaction mixture was then concentrated in vacuo and poured into water (200 mL), acidified to pH ~5 with dilute HCl and extracted with DCM (3×100 mL). The combined organic layers were dried over anhydrous Na₂SO₄ and concentrated in vacuo to afford 2-(((1r, 4r)-4-((tert-butoxycarbonyl)amino)cyclohexyl)oxy) acetic acid (Intermediate 181, 5.1 g, 75%) as an off white solid. ¹H NMR (400 MHz, CDCl₃) δ ppm 1.14-1.19 (m, 2H), 1.26-1.28 (m, 2H), 1.36-1.41 (m, 2H), 1.44 (s, 9H), 2.04-2.10 (m, 2H), 3.28-3.49 (m, 2H), 4.39 (s, 2H). Mass spec: m/z 271.9 [M−H]⁻.

### Step 3

### Intermediate 182: tert-butyl ((1r,4r)-4-(2-amino-2-oxoethoxy)cyclohexyl) carbamate

To a solution of 2-(((1r,4r)-4-((tert-butoxycarbonyl)amino)cyclohexyl)oxy)acetic acid (Intermediate 181, 1.00 g, 3.659 mmol) in DMF (10 mL) was added HATU (2.15 g, 5.488 mmol) and ammonium chloride (0.78 g, 14.64 mmol) followed by N,N-diisopropylethylamine (1.45 g, 10.98 mmol) at room temperature. The reaction mixture was stirred at room temperature for 12 h. Progress of the reaction was monitored by TLC. After completion, the reaction mixture was diluted with water (30 mL) and extracted with EtOAc (3×30 mL). The organic layer was separated, dried over anhydrous Na₂SO₄, and concentrated in vacuo to afford tert-butyl ((1r, 4r)-4-(2-amino-2-oxoethoxy)cyclohexyl)carbamate (Intermediate 182, 600 mg crude) as a yellow solid. ¹H NMR (400 MHz, DMSO-d₆) δ ppm 1.08-1.28 (m, 4H), 1.37 (s, 9H), 1.74-1.76 (m, 2H), 1.94-1.97 (m, 2H), 3.14-3.25 (m, 2H), 3.78 (s, 2H), 6.70 (d, J=7.45 Hz, 1H), 7.01 (br s, 1H), 7.20 (br s, 1H).

### Step 4

### Intermediate 183: tert-butyl 6-(2-(((1r,4r)-4-((tert-butoxycarbonyl)amino)cyclohexyl)oxy)acetamido)-2-((R)-1-methylpyrrolidin-2-yl)-1H-pyrrolo[3,2-c]pyridine-1-carboxylate

To a solution of tert-butyl (R)-6-bromo-2-(1-methylpyrrolidin-2-yl)-1H-pyrrolo[3,2-c]pyridine-1-carboxylate (Intermediate 182, 0.40 g, 1.052 mmol) and tert-butyl ((1r,4r)-4-(2-amino-2-oxoethoxy)cyclohexyl)carbamate (Intermediate 4, 0.22 g, 0.84 mmol) and in 1,4-dioxane (15.0 mL) was added Cesium carbonate (1.03 g, 3.15 mmol) and reaction mixture was degassed with Argon gas for 10 min. Pd₂(dba)₃ (0.14 g, 0.15 mmol) was added, followed by Xantphos (0.18 g, 0.315 mmol) and the mixture degassed with Argon for 5 min. The reaction mixture was then heated at 130° C. for 2 h. The reaction mixture was concentrated in vacuo and the resulting crude material was purified by combi-flash chromatography (50% EtOAc in heptane) to afford tert-butyl 6-(2-(((1r,4r)-4-((tert-butoxycarbonyl)amino)cyclohexyl)oxy)acetamido)-2-((R)-1-methylpyrrolidin-2-yl)-1H-pyrrolo[3,2-c]pyridine-1-carboxylate (Intermediate 183, 0.32 g, 53%) as an off white solid. ¹H NMR (400 MHz, DMSO-d₆) δ ppm 1.12-1.32 (m, 6H), 1.37 (s, 9H), 1.49-1.63 (m, 2H), 1.67 (s, 9H), 1.70-1.84 (m, 4H), 1.97-2.07 (m, 2H), 2.34 (s, 3H), 3.09-3.15 (m, 1H), 3.20-3.24 (m, 1H), 3.88-3.92 (m, 1H), 4.12 (s, 2H), 6.70 (s, 1H) 6.72 (s, 1H), 8.52 (s, 1H), 8.79 (s, 1H), 9.50 (s, 1H). Mass spec: m/z: 571.9 [M+H]⁺.

### Step 5

### Intermediate 184: 2-(((1r, 4r)-4-aminocyclohexyl)oxy)-N-(2-((R)-1-methylpyrrolidin-2-yl)-1H-pyrrolo[3,2-c]pyridin-6-yl) acetamide dihydrochloride

To a solution of tert-butyl 6-(2-(((1r,4R)-4-((tert-butoxycarbonyl)amino)cyclohexyl)oxy)acetamido)-2-((R)-1-methylpyrrolidin-2-yl)-1H-pyrrolo[3,2-c]pyridine-1-carboxylate (Intermediate 183, 0.30 g, 0.52 mmol) in 1,4-dioxane (5.0 mL) was added HCl (4M in 1,4-dioxane, 5.0 mL) at 0° C. The reaction mixture was stirred at room temperature for 16 h then concentrated in vacuo to afford 2-(((1r,4r)-4-aminocyclohexyl)oxy)-N-(2-((R)-1-methylpyrrolidin-2-yl)-1H-pyrrolo[3,2-c]pyridin-6-yl) acetamide dihydrochloride (Intermediate 184, 0.25 g) as an off white solid, which was used in the next step without further purification. ¹H NMR (400 MHz, DMSO-d₆) δ ppm 1.30-1.46 (m, 4H), 1.91-1.99 (m, 4H), 2.09-2.24 (m, 4H), 2.32-2.40 (m, 2H), 2.79 (s, 3H), 2.99-3.05 (m, 1H), 3.15-3.33 (m, 1H), 3.39-3.47 (m, 1H), 4.26 (s, 2H), 7.21 (s, 1H), 8.08 (s, 2H), 8.16 (s, 1H), 8.99 (s, 1H), 11.03 (br s, 1H), 11.43 (br s, 1H). Mass spec: m/z 372 [M+H]⁺.

### Step 6

### Example 39: 2-(((1r,4r)-4-((2-(2,6-dioxopiperidin-3-yl)-1,3-dioxoisoindolin-4-yl)amino)cyclohexyl)oxy)-N-(2-((R)-1-methylpyrrolidin-2-yl)-1H-pyrrolo[3,2-c]pyridin-6-yl)acetamide

To a solution of 2-(((1r,4r)-4-aminocyclohexyl)oxy)-N-(2-((R)-1-methylpyrrolidin-2-yl)-1H-pyrrolo[3,2-c]pyridin-6-yl) acetamide dihydrochloride (Intermediate 184, 0.25 g, 0.67 mmol) and 2-(2,6-dioxopiperidin-3-yl)-4-fluoroisoindoline-1,3-dione (CAS No: 835616-60-9, 0.18 g, 0.67 mmol) in DMSO (1 mL) was added diisopropylethylamine (0.44 g, 3.36 mmol) and the reaction mixture heated at 130° C. for 2 h. The reaction mixture was then poured into ice cold water, resulting a solid precipitate, which was filtered and dried under vacuum. The crude solid was purified by preparative HPLC (Method A), pure fractions were collected and lyophilised to afford 2-(((1r,4r)-4-((2-(2,6-dioxopiperidin-3-yl)-1,3-dioxoisoindolin-4-yl)amino)cyclohexyl)oxy)-N-(2-((R)-1-methylpyrrolidin-2-yl)-1H-pyrrolo[3,2-c]pyridin-6-yl)acetamide (Example 39, 0.042 g, 10%) as a yellow solid. ¹H NMR (400 MHz, DMSO-d₆) δ ppm 1.32-1.40 (m, 2H), 1.44-1.53 (m, 2H), 1.77-1.89 (m, 3H), 2.02-2.09 (m, 6H), 2.15 (s, 3H), 2.22-2.28 (m, 1H), 2.52-2.54 (m, 1H) 2.84-2.92 (m, 1H), 3.11-3.14 (m, 1H), 3.28-3.32 (m, 2H), 3.47-3.54 (m, 1H), 3.60-3.63 (m, 1H), 4.13 (s, 2H), 5.01-5.06 (m, 1H), 6.20 (d, J=8.25 Hz, 1H), 6.37 (s, 1H), 7.04 (d, J=7.00 Hz, 1H), 7.20 (d, J=8.63 Hz, 1H), 7.59-7.60 (m, 1H), 8.08 (s, 1H), 8.43 (s, 1H), 9.31 (br s, 1H), 11.08 (br s, 1H), 11.34 (br s, 1H). Mass spec: m/z 628.4 [M+H]⁺.

### Example 40 was Synthesised Following Scheme 38

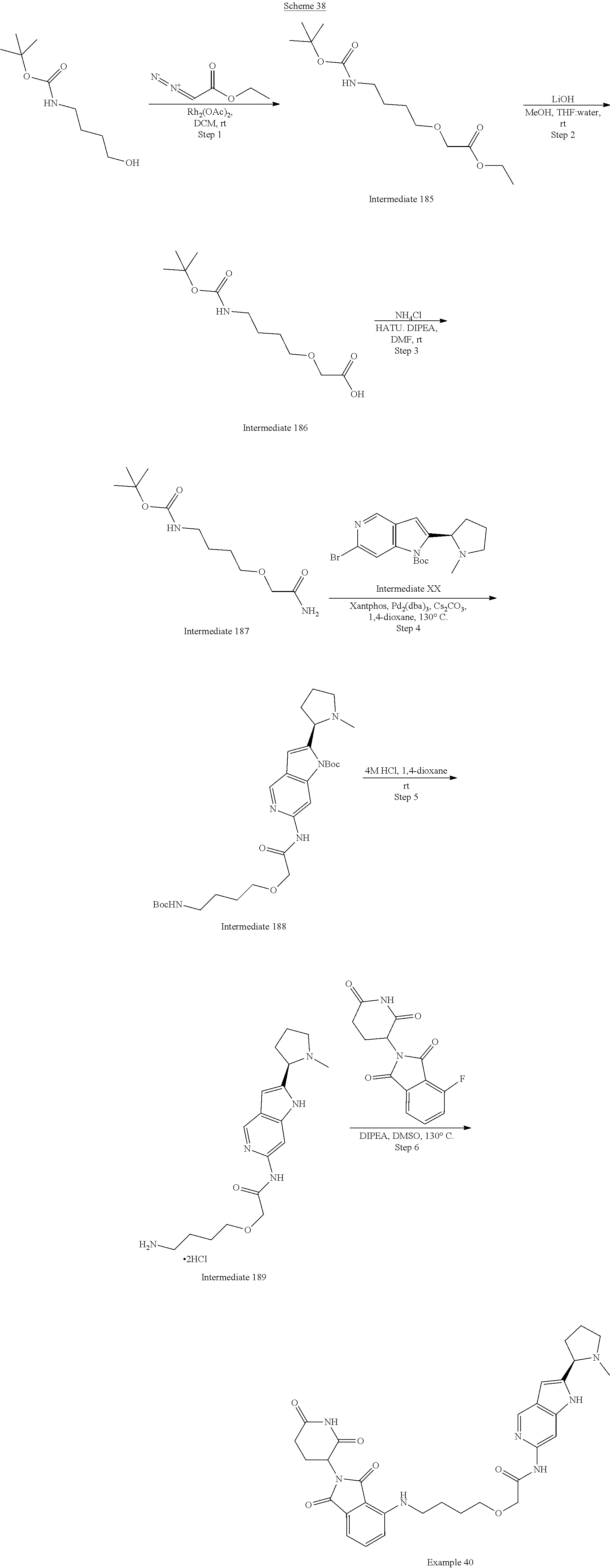

### Step 1

### Intermediate 185: ethyl 2-(4-((tert-butoxycarbonyl)amino)butoxy)acetate

To a solution of tert-butyl (4-hydroxybutyl) carbamate (10.0 g, 52.84 mmol) in dichloromethane (100 mL) was added rhodium (ii) acetate dimer (1.18 g, 2.64 mmol) at room temperature. The reaction mixture was stirred for 10 min then ethyl 2-diazoacetate (7.23 g, 63.40 mmol) was added dropwise at room temperature. The reaction mixture was stirred at room temperature for 24 h. Progress of the reaction was monitored by TLC. After completion, the reaction mixture was poured into water (100 mL) extracted with DCM (3×100 mL). The organic layer was separated, dried over anhydrous Na₂SO₄ and concentrated in vacuo. The crude material was purified by combi-flash column chromatography by eluting with 20% EtOAc in heptane to afford ethyl 2-(4-((tert-butoxycarbonyl)amino) butoxy)acetate (Intermediate 185, 8.0 g, 55%) as an off white solid. ¹H NMR (400 MHz, DMSO-d₆) δ ppm 1.17-1.21 (m, 3H), 1.38 (s, 9H), 1.45-1.49 (m, 4H), 2.88-2.93 (m, 2H), 3.40-3.44 (m, 2H), 4.05 (s, 2H), 4.08-4.14 (m, 2H).

### Step 2

### Intermediate 186: 2-(4-((tert-butoxycarbonyl)amino)butoxy) acetic acid

To a solution of ethyl 2-(4-((tert-butoxycarbonyl)amino)butoxy)acetate (Intermediate 185, 2.5 g, 0.10 mmol) in tetrahydrofuran (20 mL) and methanol (10 mL) was added lithium hydroxide (0.72 g, 0.03 mmol) in water (10 mL) at 0° C. The reaction mixture was stirred at room temperature for 24 h. Progress of the reaction was monitored by TLC. After completion, the reaction mixture was concentrated in vacuo, and diluted with water (20 mL), pH was adjusted to 3-4 with saturated solution of citric acid (15 mL) and extracted with EtOAc (2×50 mL). The organic layer was separated, dried over anhydrous Na₂SO₄ and concentrated in vacuo. The crude material was purified by combi-flash column chromatography by eluting with 50% EtOAc in heptane to afford 2-(4-((tert-butoxycarbonyl)amino)butoxy) acetic acid (Intermediate 186, 2.2 g, 88%) as a yellow liquid. ¹H NMR (400 MHz, DMSO-d₆) δ ppm 1.37 (s, 9H), 1.41-1.49 (m, 4H), 2.88-2.93 (m, 2H), 3.31-3.43 (m, 2H), 3.96 (s, 2H), 6.77 (br s, 1H), 12.46 (br s, 1H).

### Step 3

### Intermediate 187: tert-butyl (4-(2-amino-2-oxoethoxy)butyl)carbamate

To a solution of 2-(4-((tert-butoxycarbonyl)amino)butoxy)acetic acid (Intermediate 186, 2.2 g, 9.4 mmol) in DMF (2 mL) was added diisopropylethylamine (8.2 mL, 47.0 mmol) followed HATU (5.5 g, 14.0 mmol) and ammonium chloride (2.5 g, 47.0 mmol). The reaction mixture was stirred at room temperature for 12 h. Progress of the reaction was monitored by TLC. After completion, the reaction mixture was diluted with water (50 mL) and extracted with EtOAc (3×50 mL). The organic layer was separated, dried over anhydrous Na₂SO₄ and concentrated in vacuo. The crude material was purified by combi-flash column chromatography by eluting with 80% EtOAc in heptane to afford tert-butyl (4-(2-amino-2-oxoethoxy)butyl)carbamate (Intermediate 187, 1.8 g, 82%) as a yellow solid. ¹H NMR (400 MHz, DMSO-d₆) 1.35 (s, 9H), 1.44-1.50 (m, 4H), 2.91-2.93 (m, 2H), 3.36-3.42 (m, 2H), 4.02 (s, 2H) 6.68 (s, 1H), 7.40 (s, 1H), 7.60 (d, J=8.32 Hz, 1H).

### Step 4

### Intermediate 188: tert-butyl (R)-6-(2-(4-((tert-butoxycarbonyl)amino)butoxy)acetamido)-2-(1-methylpyrrolidin-2-yl)-1H-pyrrolo[3,2-c]pyridine-1-carboxylate

To a solution of tert-butyl (R)-6-bromo-2-(1-methylpyrrolidin-2-yl)-1H-pyrrolo[3,2-c]pyridine-1-carboxylate (Intermediate 4, 0.40 g, 1.05 mmol) in 1,4-dioxane was added tert-butyl (4-(2-amino-2-oxoethoxy)butyl) carbamate (Intermediate 187, 0.21 g, 0.80 mmol) (15 mL) followed by cesium carbonate (1.03 g, 3.15 mmol). The reaction mixture was purged with argon for 10 min then added Pd₂(dba)₃ (0.15 g, 0.15 mmol) and Xantphos (0.18 g, 0.31 mmol) at room temperature. The reaction mixture was again purged with argon gas and heated at 130° C. for 2 h. Progress of the reaction was monitored by TLC. After completion, the reaction mixture was concentrated in vacuo. The crude material was purified by combi-flash column chromatography by eluting with 95% EtOAc in heptane to tert-butyl (R)-6-(2-(4-((tert-butoxycarbonyl)amino)butoxy)acetamido)-2-(1-methylpyrrolidin-2-yl)-1H-pyrrolo[3,2-c]pyridine-1-carboxylate (Intermediate 188, 0.3 g, 52%) as an off-white solid. Mass spec: m/z 546.4 [M+H]⁺.

### Step 5

### Intermediate 189: (R)-2-(4-aminobutoxy)-N-(2-(1-methylpyrrolidin-2-yl)-1H-pyrrolo[3,2-c]pyridin-6-yl) acetamide dihydrochloride

To a solution of solution of tert-butyl (R)-6-(2-(4-((tert-butoxycarbonyl)amino) butoxy) acetamido)-2-(1-methylpyrrolidin-2-yl)-1H-pyrrolo[3,2-c]pyridine-1-carboxylate (Intermediate 188, 0.28 g, 0.51 mmol) in 1,4-dioxane (5.0 mL) was added 4M HCl in 1,4-dioxane (5.0 mL) at 0° C. The reaction mixture was stirred at room temperature for 16 h. Progress of the reaction was monitored by TLC. After completion, the reaction mixture was concentrated in vacuo to afford (R)-2-(4-aminobutoxy)-N-(2-(1-methylpyrrolidin-2-yl)-1H-pyrrolo[3,2-c]pyridin-6-yl) acetamide dihydrochloride (Intermediate 189, 0.22 g) as an off-white solid which was used in the next step without further purification.

### Step 6

### Example 40: 2-(4-((2-(2,6-dioxopiperidin-3-yl)-1,3-dioxoisoindolin-4-yl)amino)butoxy)-N-(2-((R)-1-methylpyrrolidin-2-yl)-1H-pyrrolo[3,2-c]pyridin-6-yl)acetamide

To a solution of (R)-2-(4-aminobutoxy)-N-(2-(1-methylpyrrolidin-2-yl)-1H-pyrrolo[3,2-c]pyridin-6-yl)acetamide dihydrochloride (Intermediate 189, 0.22 g, 0.22 mmol) and 2-(2,6-dioxo-3-piperidyl)-4-fluoro-isoindoline-1,3-dione (CAS No: 835616-60-9, 0.18 g, 0.64 mmol) in dimethyl sulfoxide (1.0 mL) was added N,N-Diisopropylethylamine (0.42 g, 3.20 mmol). The reaction mixture was heated at 130° C. for 2 h. Progress of reaction was monitored by TLC. After completion, reaction mixture was poured into ice cold water (15 mL), yellow solid precipitated, which was filtered, dried to obtain crude compound. The crude material was purified by preparative HPLC (Method A) to afford 2-(4-((2-(2,6-dioxopiperidin-3-yl)-1,3-dioxoisoindolin-4-yl)amino)butoxy)-N-(2-((R)-1-methylpyrrolidin-2-yl)-1H-pyrrolo[3,2-c]pyridin-6-yl)acetamide (Example 40, 0.02 g, 6%) as a yellow solid. ¹H NMR (400 MHz, DMSO-d₆) δ ppm 1.64-1.72 (m, 4H), 1.75-1.94 (m, 3H), 1.98-2.06 (m, 1H), 2.10-2.12 (m, 1H), 2.15 (s, 3H), 2.21-2.26 (m, 1H), 2.54-2.62 (m, 2H), 2.83-2.92 (m, 1H), 3.10-3.14 (m, 1H), 3.25-3.28 (m, 1H), 3.30-3.38 (m, 2H), 3.56-3.60 (m, 2H), 4.09 (s, 2H), 5.02-5.07 (m, 1H), 6.36 (br s, 1H), 6.60 (t, J=5.75 Hz, 1H), 7.01 (d, J=7.00 Hz, 1H), 7.14 (d, J=8.50 Hz, 1H), 7.54-7.59 (m, 1H), 8.07 (s, 1H), 8.41 (s, 1H), 9.44 (br s, 1H), 11.08 (br s, 1H), 11.32 (br s, 1H). Mass spec: m/z 602.2 [M+H]⁺.

### Example 41 was Synthesised Following Scheme 39

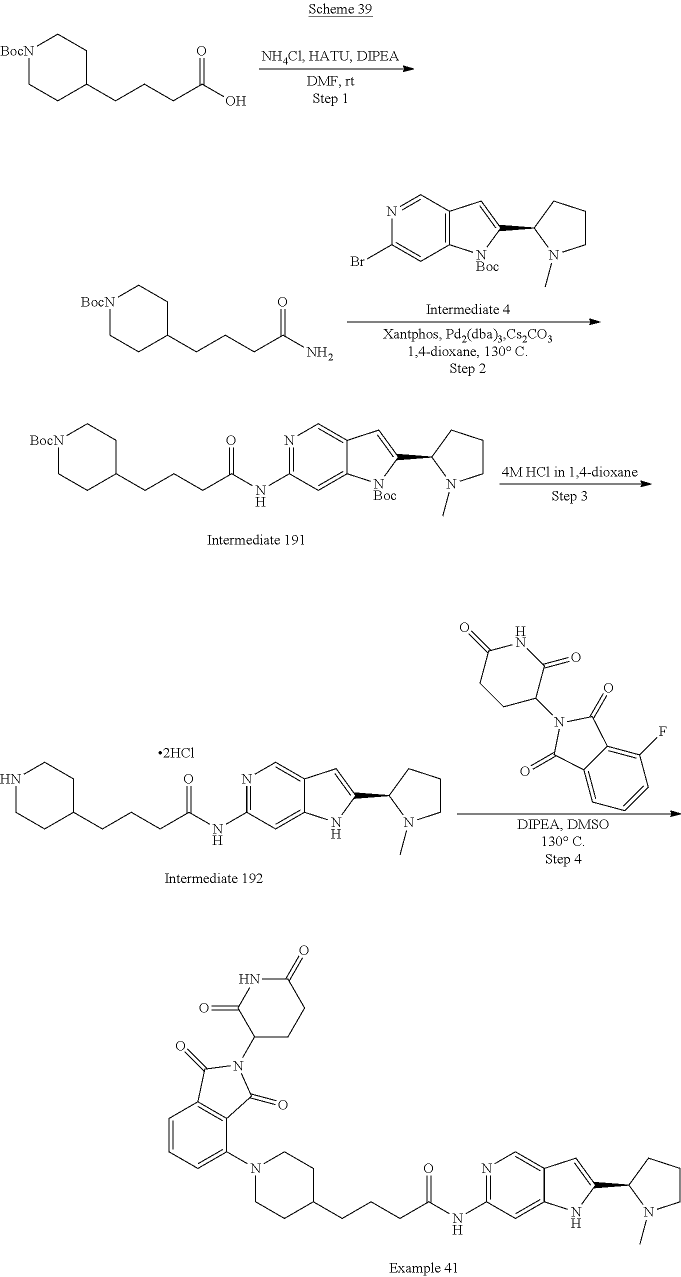

### Step 1

### Intermediate 190: tert-butyl 4-(4-amino-4-oxobutyl) piperidine-1-carboxylate

To a solution of 4-(1-(tert-butoxycarbonyl)piperidin-4-yl)butanoic acid (CAS No: 142247-38-9, 2 g, 7 mmol) in DMF (20 mL) was added HATU (1.5 g, 3.8 mmol) followed by NH₄Cl (4.0 g, 75 mmol) and N,N-Diisopropylethylamine (3 g, 20 mmol) at room temperature. The reaction mixture was stirred at room temperature for 12 h. The reaction mixture was then diluted with water (100 mL) and extracted with EtOAc (3×100 mL). The organic layer was separated, dried over anhydrous Na₂SO₄ and concentrated in vacuo. The crude material was purified by combi-flash column chromatography, by eluting with 50% EtOAc in heptane, to afford tert-butyl 4-(4-amino-4-oxobutyl) piperidine-1-carboxylate (Intermediate 190, 1.5 g, 51%) as Colorless Liquid. ¹H NMR (400 MHz, DMSO-d₆) δ ppm 0.87-0.97 (m, 2H), 1.13-1.19 (m, 2H), 1.22-1.27 (m, 1H), 1.38 (s, 9H), 1.45-1.54 (m, 2H), 1.59-1.62 (m, 2H) 1.99-2.02 (m, 2H) 2.66-2.70 (m, 2H), 3.88-3.92 (m, 2H), 6.66 (s, 1H), 7.20 (br s, 1H). Mass spec: m/z 171.2 [M-Boc]+.

### Step 2

### Intermediate 191: tert-butyl (R)-6-(4-(1-(tert-butoxycarbonyl)piperidin-4-yl)butanamido)-2-(1-methylpyrrolidin-2-yl)-1H-pyrrolo[3,2-c]pyridine-1-carboxylate

To stirred a solution of tert-butyl (R)-6-bromo-2-(1-methylpyrrolidin-2-yl)-1H-pyrrolo[3,2-c]pyridine-1-carboxylate (Intermediate 4, 0.4 g, 1.0 mmol) in 1,4-dioxane (20.0 mL) was added tert-butyl 4-(4-amino-4-oxobutyl)piperidine-1-carboxylate (Intermediate 190, 0.2 g, 0.8 mmol) and cesium carbonate (1.0 g, 0.3 mmol) at room temperature. The reaction mixture was purged with argon for 10 min then Pd₂(dba)₃ (0.1 g, 0.1 mmol) and 4,5-bis(diphenylphosphino)-9,9-dimethylxanthene (0.2 g, 0.3 mmol) were added at room temperature and the reaction mixture was then stirred at 130° C. for 2 h. The reaction mixture was then concentrated in vacuo and the resulting residue was purified by combi-flash column chromatography, by eluting with 80% EtOAc in heptane, to afford tert-butyl (R)-6-(4-(1-(tert-butoxycarbonyl)piperidin-4-yl)butanamido)-2-(1-methylpyrrolidin-2-yl)-1H-pyrrolo[3,2-c]pyridine-1-carboxylate (Intermediate 191, 0.3 g, 50%) as a grey solid. ¹H NMR (400 MHz, DMSO-d₆) δ ppm 0.92-0.98 (m, 2H), 1.18-1.27 (m, 2H), 1.38 (s, 9H), 1.57-1.61 (m, 7H), 1.66 (s, 9H), 1.69-1.79 (m, 3H), 2.27 (s, 3H) 2.30-2.42 (m, 3H), 2.59-2.74 (m, 2H), 3.08-3.16 (m, 1H), 3.89-3.90 (m, 2H), 6.70 (s, 1H), 8.49 (s, 1H), 8.79 (s, 1H), 10.32 (br s, 1H). Mass spec: m/z 570.5 [M+H]⁺.

### Step 3

### Intermediate 192: (R)—N-(2-(1-methylpyrrolidin-2-yl)-1H-pyrrolo[3,2-c]pyridin-6-yl)-4-(piperidin-4-yl) butanamide dihydrochloride

To a solution of tert-butyl (R)-6-(4-(1-(tert-butoxycarbonyl)piperidin-4-yl)butanamido)-2-(1-methylpyrrolidin-2-yl)-1H-pyrrolo[3,2-c]pyridine-1-carboxylate (Intermediate 191, 0.3 g, 0.52 mmol) in 1,4-dioxane (5.0 mL) was added 4M hydrochloric acid in 1,4-dioxane (5.0 mL) at 0° C. The reaction mixture was stirred at room temperature for 16 h. The reaction mixture was then concentrated under reduced pressure to afford (R)—N-(2-(1-methylpyrrolidin-2-yl)-1H-pyrrolo[3,2-c]pyridin-6-yl)-4-(piperidin-4-yl)butanamide dihydrochloride (Intermediate 192, 0.25 g) as an off-white solid used for next step without purification. ¹H NMR (400 MHz, DMSO-d₆) δ ppm 1.23-1.34 (m, 4H), 1.55-1.61 (m, 1H), 1.63-1.70 (m, 2H), 1.79-1.82 (m, 2H), 2.15-2.20 (m, 2H), 2.33-2.38 (m, 2H), 2.54 (s, 3H), 2.68-2.85 (m, 3H), 3.16-3.24 (m, 3H), 3.64-3.75 (m, 2H), 4.67-4.72 (m, 1H), 7.21 (br s, 1H), 7.84 (s, 1H), 8.93 (s, 1H), 11.40 (br s, 1H), 12.08 (br s, 1H), 13.04 (br s, 1H). Mass spec: m/z 370.3 [M+H]⁺.

### Step 4

### Example 41: 4-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)butanamide

To a solution of (R)—N-(2-(1-methylpyrrolidin-2-yl)-1H-pyrrolo[3,2-c]pyridin-6-yl)-4-(piperidin-4-yl)butanamide dihydrochloride (Intermediate 192, 0.24 g, 0.64 mmol) and 2-(2,6-dioxopiperidin-3-yl)-4-fluoroisoindoline-1,3-dione (CAS No: 835616-60-9, 0.179 g, 0.64 mmol) in dimethyl sulfoxide (1.0 mL) was added N,N-Diisopropylethylamine (0.428 g, 3.0 mmol) and the reaction mixture was heated at 130° C. for 2 h. The reaction mixture was then poured into ice cold water, a solid precipitated which was filtered and dried to obtain crude compound. The obtained crude material was purified by preparative HPLC (Method A) to afforded 4-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)butanamide (Example 41, 0.052 g, 13%) as a yellow solid. ¹H NMR (400 MHz, DMSO-d₆) δ ppm 1.26-1.38 (m, 4H), 1.42-1.49 (m, 1H), 1.61-1.71 (m, 2H), 1.75-1.83 (m, 3H), 1.84-1.93 (m, 2H), 1.98-2.07 (m, 1H), 2.08-2.13 (m, 1H), 2.14 (s, 3H), 2.21-2.28 (m, 1H), 2.38 (t, J=7.25 Hz, 2H), 2.53-2.62 (m, 2H), 2.82-2.90 (m, 2H), 3.09-3.15 (m, 1H), 3.25-3.29 (m, 2H), 3.69-3.70 (m, 2H), 5.06-5.10 (m, 1H), 6.33 (s, 1H), 7.30-7.32 (m, 2H), 7.64-7.68 (m, 1H), 8.08 (s, 1H), 8.40 (s, 1H), 10.12 (s, 1H), 11.06 (br s, 1H), 11.24 (br s, 1H). Mass spec: m/z 626.5 [M+H]⁺.

### Example 42 was Prepared Following Scheme 40

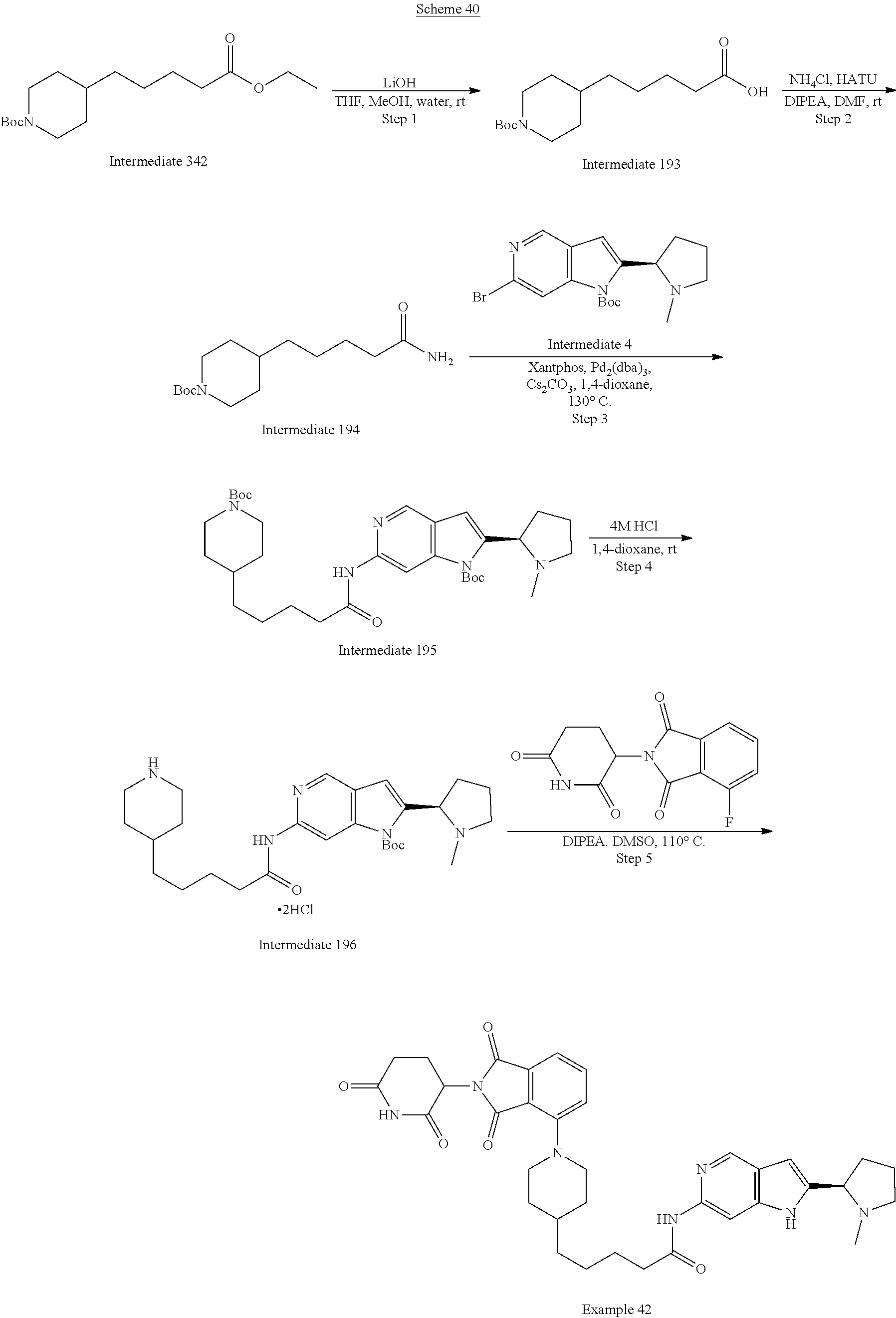

### Step 1

### Intermediate 193: 5-(1-tert-Butoxycarbonyl-4-piperidyl)pentanoic acid

To a tert-butyl 4-(5-ethoxy-5-oxopentyl)piperidine-1-carboxylate (Intermediate 342, 2.50 g, 8.4 mmol) in methanol (8 mL) and THF (8 mL) was added lithium hydroxide (0.60 g, 25 mmol) in water (10 mL). The reaction mixture was stirred at room temperature for 6 h. Progress of the reaction was monitored by TLC. After completion, the reaction mixture was concentrated in vacuo. The reaction mixture was diluted with water (50 mL), acidified to ~pH5 using aqueous 1 N HCl and extracted with EtOAc (2×100 mL). The combined organic layer was dried over Na₂SO₄ and concentrated in vacuo to afford 5-(1-tert-butoxycarbonyl-4-piperidyl)pentanoic acid (Intermediate 193, 2.30 g) as a liquid which was used for the next step without further purification. ¹H NMR (400 MHz, DMSO-d₆) δ ppm 0.87-0.97 (m, 2H), 1.15-1.23 (m, 2H), 1.25-1.31 (m, 2H), 1.38 (s, 9H), 1.42-1.51 (m, 2H), 1.53-1.65 (m, 2H), 2.19 (t, J=7.23 Hz, 2H), 2.65 (s, 2H), 3.31 (s, 1H), 3.90-3.91 (m, 2H), 11.97 (br s, 1H). Mass spec: m/z 286.2 [M+H]⁺.

### Step 2

### Intermediate 194: tert-Butyl 4-(5-amino-5-oxopentyl) piperidine-1-carboxylate

To a solution of 5-(1-tert-butoxycarbonyl-4-piperidyl) pentanoic acid (Intermediate 193, 2.30 g, 8.06 mmol) in DMF (15 mL) was added HATU (4.74 g, 12.1 mmol) and N,N-diisopropylethylamine (3.19 g, 24.2 mmol) followed by ammonium chloride (1.73 g, 32.2 mmol) at room temperature. The reaction mixture was stirred at room temperature for 16 h. Progress of the reaction was monitored by TLC. After completion, the reaction mixture was quenched with ice cold water (80 mL), extracted with EtOAc (2×100 mL). The organic layer was separated, washed with water (100 mL), brine solution (100 mL), dried over Na₂SO₄ and concentrated in vacuo. The crude material was purified by combi-flash column chromatography, by eluting with 90% EtOAc in heptane, to afford tert-butyl 4-(5-amino-5-oxopentyl) piperidine-1-carboxylate (Intermediate 194, 1.40 g, 61%) as a brownish-white solid. ¹H NMR (400 MHz, DMSO-d₆) δ ppm 0.87-0.97 (m, 2H), 1.14-1.30 (m, 4H), 1.38 (s, 9H), 1.41-1.50 (m, 2H), 1.53-1.65 (m, 2H), 2.02 (t, J=7.34 Hz, 2H), 2.55-2.73 (m, 3H), 3.90-3.91 (m, 2H), 6.66 (br s, 1H), 7.20 (br s, 1H). Mass spec: m/z 285.2 [M+H]⁺.

### Step 3

### Intermediate 195: tert-Butyl (R)-6-(5-(1-(tert-butoxycarbonyl) piperidin-4-yl) pentanamido)-2-(1-methylpyrrolidin-2-yl)-1H-pyrrolo[3,2-c]pyridine-1-carboxylate

To a solution of tert-butyl 4-(5-amino-5-oxopentyl) piperidine-1-carboxylate (Intermediate 194, 0.23 g, 0.84 mmol) and tert-butyl (R)-6-bromo-2-(1-methylpyrrolidin-2-yl)-1H-pyrrolo[3,2-c]pyridine-1-carboxylate (Intermediate 4, 0.40 g, 1.05 mmol) in 1,4-dioxane (5 mL) was added cesium carbonate (1.03 g, 3.15 mmol). The reaction mixture was purged with argon for 20 min. To the reaction mixture was added Pd₂(dba)₃, (0.12 g, 0.13 mmol) followed by 4,5-bis(diphenylphosphino)-9,9 dimethylxanthene (0.18 g, 0.31 mmol) and the reaction mixture was purged with argon for 10 min. The reaction mixture was heated at 130° C. for 5 h. Progress of the reaction was monitored by TLC. After completion of the reaction, the reaction mixture was concentrated in vacuo. The crude material was purified by combi-flash column chromatography, by eluting with 85% EtOAc in heptane, to afford tert-butyl (R)-6-(5-(1-(tert-butoxycarbonyl) piperidin-4-yl) pentanamido)-2-(1-methylpyrrolidin-2-yl)-1H-pyrrolo[3,2-c]pyridine-1-carboxylate (Intermediate 195, 0.39 g, 64%) as an off-white solid. ¹H NMR (400 MHz, DMSO-d₆) δ ppm 0.87-0.96 (m, 2H), 1.18-1.25 (m, 2H), 1.25-1.35 (m, 3H), 1.37 (s, 9H), 1.55-1.63 (m, 4H), 1.66 (s, 9H), 1.67-1.80 (m, 3H), 2.26 (s, 3H), 2.27-2.43 (m, 4H), 2.57-2.70 (m, 2H), 3.05-3.15 (m, 1H), 3.79-3.99 (m, 3H), 6.69 (s, 1H), 8.49 (s, 1H), 8.79 (s, 1H), 10.34 (s, 1H). Mass spec: m/z 583.7 [M+H]⁺.

### Step 4

### Intermediate 196: (R)—N-(2-(1-Methylpyrrolidin-2-yl)-1H-pyrrolo[3,2-c]pyridin-6-yl)-5-(piperidin-4-yl) pentanamide dihydrochloride

To a solution of tert-butyl (R)-6-(5-(1-(tert-butoxycarbonyl) piperidin-4-yl) pentanamido)-2-(1-methylpyrrolidin-2-yl)-1H-pyrrolo[3,2-c]pyridine-1-carboxylate (Intermediate 195, 0.39 g, 0.66 mmol) in 1,4-dioxane (5 mL) was added 4 M HCl in 1,4-dioxane (2 mL) at room temperature and the reaction mixture was stirred at room temperature for 16 h. Progress of the reaction was monitored by TLC. After completion, the reaction mixture was concentrated in vacuo and washed with diethyl ether (30 mL). The crude material was triturated with diethyl ether and dried to afford (R)—N-(2-(1-Methylpyrrolidin-2-yl)-1H-pyrrolo[3,2-c]pyridin-6-yl)-5-(piperidin-4-yl) pentanamide dihydrochloride (Intermediate 196, 0.36 g) as a white solid. ¹H NMR (400 MHz, DMSO-d₆) δ ppm 1.78-1.80 (m, 2H), 2.11-2.21 (m, 2H), 2.24-2.47 (m, 5H), 2.68 (s, 3H), 2.75-2.82 (m, 2H), 3.16-3.26 (m, 5H), 3.36-3.51 (m, 7H), 4.66-4.69 (m, 1H), 7.13 (s, 1H), 7.30 (s, 1H), 7.89 (s, 1H), 8.44-8.59 (m, 1H), 8.70-8.90 (m, 2H), 11.15 (s, 1H), 12.78 (s, 1H). Mass spec: m/z 383.6 [M+H]⁺.

### Step 5

### Example 42: 5-(1-(2-(2,6-Dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)pentanamide

To a solution of (R)—N-(2-(1-Methylpyrrolidin-2-yl)-1H-pyrrolo[3,2-c]pyridin-6-yl)-5-(piperidin-4-yl) pentanamide dihydrochloride (Intermediate 196, 0.36 g, 0.93 mmol) in DMSO (3 mL) was added 2-(2,6-dioxopiperidin-3-yl)-4-fluoroisoindoline-1,3-dione (CAS No: 835616-60-9, 0.27 g, 0.93 mmol) followed by N,N-diisopropylethylamine (0.61 g, 1.22 mmol). The reaction mixture was heated at 110° C. for 6 h. Progress of the reaction was monitored by TLC. After completion, the reaction mixture was cooled to room temperature, treated with cold water (50 mL) to get a yellow precipitate, filtered, and then dried. The crude compound was purified by preparative HPLC (Method A) to afford 5-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)pentanamide (Example 42, 0.07 g, 12%) as a yellow solid. ¹H NMR (400 MHz, DMSO-d₆) δ ppm 1.27-1.42 (m, 7H), 1.54-1.65 (m, 2H), 1.73-1.91 (m, 5H), 1.98-2.06 (m, 2H), 2.14 (s, 3H), 2.21-2.28 (m, 1H), 2.38 (t, J=7.27 Hz, 2H), 2.56-2.64 (m, 1H), 2.80-2.92 (m, 4H), 3.07-3.17 (m, 1H), 3.28 (s, 1H), 3.68-3.70 (m, 2H), 5.08-5.09 (m, 1H), 6.33-6.34 (m, 1H), 7.31-7.32 (m, 2H), 7.66-7.68 (m, 1H), 8.08 (s, 1H), 8.40 (s, 1H), 10.11 (s, 1H), 11.07 (s, 1H), 11.24 (s, 1H). Mass spec: m/z 640.3 [M+H]⁺.

### Example 43 was Synthesised Following Scheme 41

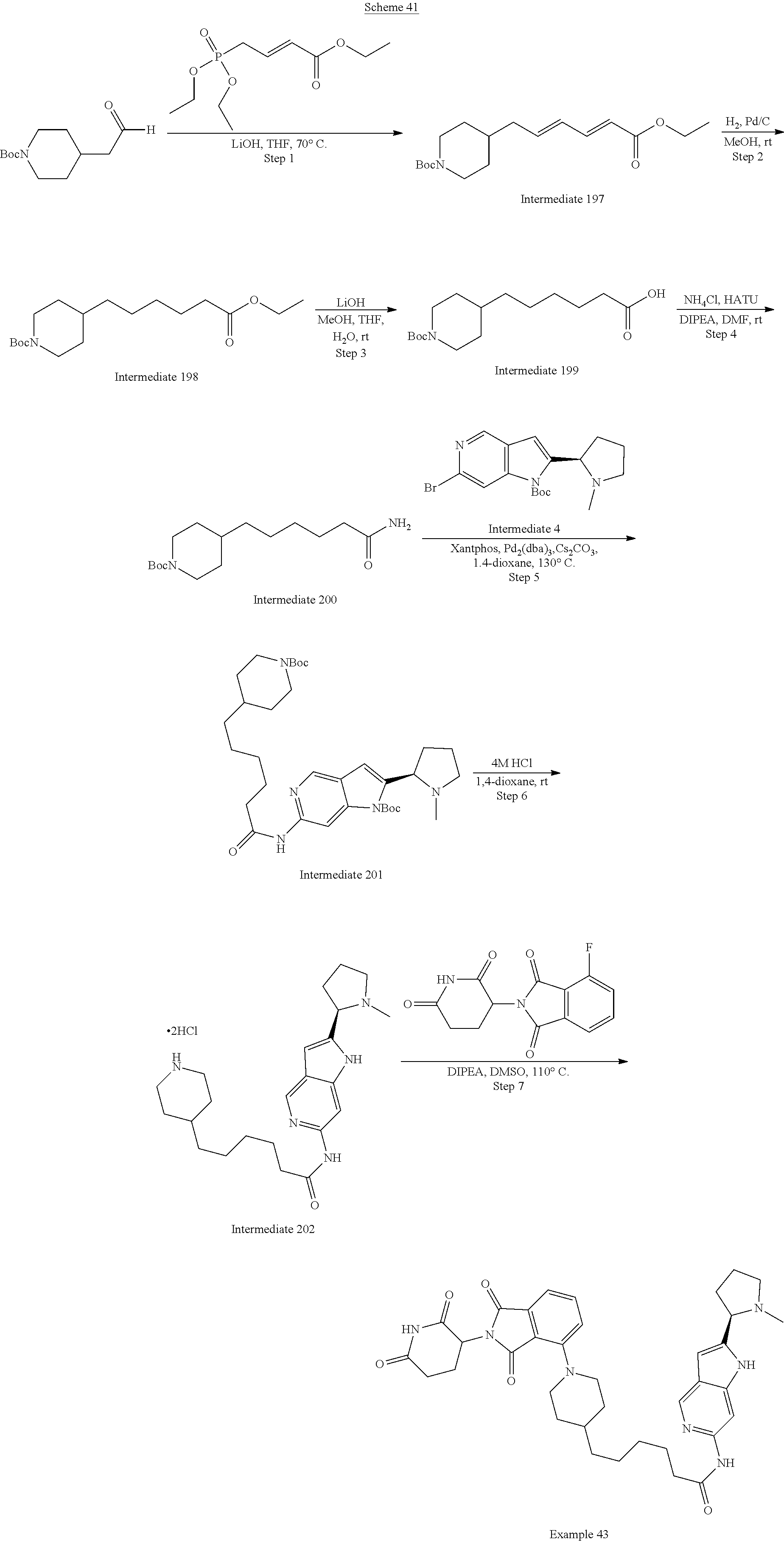

### Step 1

### Intermediate 197: tert-butyl 4-((2E,4E)-6-ethoxy-6-oxohexa-2,4-dien-1-yl) piperidine-1-carboxylate

To a solution of tert-butyl 4-(2-oxoethyl)piperidine-1-carboxylate (CAS No: 142374-19-4, 5.00 g, 21.99 mmol) and triethyl 4-phosphonocrotonate (6.12 g, 21.99 mmol) in tetrahydrofuran (50 mL) was added lithium hydroxide (1.07 g, 43.99 mmol) and the reaction mixture was heated at 70° C. for 2 h. The reaction mixture was then quenched with water (100 mL) and extracted with EtOAc (3×100 mL). The combined organic layers were separated and washed with water (100 mL) then brine solution (100 mL), dried over Na₂SO₄ and concentrated in vacuo. The crude material was purified by combi-flash column chromatography, by eluting with 90% EtOAc in heptane, to afford tert-butyl 4-((2E,4E)-6-ethoxy-6-oxohexa-2,4-dien-1-yl)piperidine-1-carboxylate (Intermediate 197, 3.80 g, 53%) as a colourless liquid. ¹H NMR (400 MHz, DMSO-d₆) δ ppm 0.94-1.04 (m, 2H), 1.21 (t, J=7.02 Hz, 3H), 1.38 (s, 9H), 1.53-1.61 (m, 2H), 2.09 (t, J=5.48 Hz, 2H), 2.63-2.69 (m, 2H), 3.65-3.67 (m, 1H), 3.89-3.92 (m, 2H), 4.09-4.14 (m, 2H), 5.85-5.91 (m, 1H), 6.22-6.28 (m, 2H), 7.19-7.26 (m, 1H). Mass spec: m/z 267.6 [M+H-t-butyl]+.

### Step 2

### Intermediate 198: tert-butyl 4-(6-ethoxy-6-oxohexyl) piperidine-1-carboxylate

To a solution of tert-butyl 4-((2E,4E)-6-ethoxy-6-oxohexa-2,4-dien-1-yl) piperidine-1-carboxylate (Intermediate 197, 3.80 g, 12 mmol) in methanol (60 mL) was added Pd/C (10% by wt., 1.20 g, 11 mmol) and the reaction mixture stirred at room temperature under 70 psi hydrogen pressure for 16 h. The reaction mixture was then filtered through a celite bed and washing with methanol. The solvent was evaporated under reduced pressure to afford tert-butyl 4-(6-ethoxy-6-oxohexyl)piperidine-1-carboxylate (Intermediate 198, 3.40 g) as colourless oil which was used for the next step without further purification. ¹H NMR (400 MHz, DMSO-d₆) δ ppm 0.90-0.95 (m, 3H), 1.15-1.21 (m, 3H), 1.23-1.29 (m, 4H), 1.39 (s, 9H), 1.48-1.55 (m, 2H), 1.59-1.62 (m, 2H), 2.26-2.29 (m, 2H), 2.63-2.66 (m, 2H), 3.56-3.59 (m, 2H), 3.89-3.92 (m, 2H), 4.02-4.07 (m, 2H).

### Step 3

### Intermediate 199: 6-(1-(tert-butoxycarbonyl) piperidin-4-yl) hexanoic acid

To a solution of tert-butyl 4-(6-ethoxy-6-oxohexyl) piperidine-1-carboxylate (Intermediate 198, 3.40 g, 10 mmol) in MeOH (10 mL) and THF (10 mL) was added lithium hydroxide (0.50 g, 21 mmol) in H₂O (10 mL). and the reaction mixture stirred at room temperature for 6 h. The reaction mixture was then diluted with water (60 mL), acidified to pH~5 using aqueous 1N HCl and extracted with EtOAc (2×100 mL). The combined organic layer was separated, dried over Na₂SO₄ and concentrated in vacuo to afford 6-(1-(tert-butoxycarbonyl) piperidin-4-yl) hexanoic acid (Intermediate 199, 3.05 g) as a liquid which was used for the next step without further purification. ¹H NMR (400 MHz, DMSO-d₆) δ ppm 0.90-0.94 (m, 2H), 1.16-1.25 (m, 6H), 1.32-1.34 (m, 1H), 1.38 (s, 9H), 1.45-1.54 (m, 2H), 1.59-1.62 (m, 2H), 2.18 (t, J=7.30 Hz, 2H), 2.63-2.65 (m, 2H), 3.89-3.92 (m, 2H), 11.95 (br s, 1H). Mass spec: m/z 300.3 [M+H]⁺.

### Step 4

### Intermediate 200: tert-butyl 4-(6-amino-6-oxohexyl) piperidine-1-carboxylate

To a solution of 6-(1-(tert-butoxycarbonyl)piperidin-4-yl)hexanoic acid (Intermediate 199, 3.20 g, 10 mmol) in DMF (25 mL) was added HATU (6.0 g, 15 mmol) and diisopropylethylamine (5.3 mL, 30 mmol) and the reaction mixture was stirred at room temperature for 15 min. Ammonium chloride (2.2 g, 41 mmol) was then added and the reaction stirred at room temperature and stirred for 16 h. The reaction mixture was then quenched with water (200 mL), extracted with EtOAc (3×100 mL). The combined organic layers were then washed with H₂O (200 mL), brine solution (200 mL), dried over Na₂SO₄ and concentrated in vacuo. The crude material was purified by combi-flash column chromatography, by eluting with 95% EtOAc in heptane, to afford tert-butyl 4-(6-amino-6-oxohexyl) piperidine-1-carboxylate (Intermediate 200, 2.50 g, 83%) as an off white solid. ¹H NMR (400 MHz, DMSO-d₆) δ ppm 0.86-0.97 (m, 2H), 1.15-1.23 (m, 4H), 1.38 (s, 9H), 1.43-1.50 (m, 2H), 1.59-1.62 (m, 2H), 1.99-2.03 (m, 3H), 2.65-2.73 (m, 3H), 3.88-3.92 (m, 2H), 4.00-4.05 (m, 1H), 6.65 (br s, 1H) 7.19 (br s, 1H). Mass spec: m/z 299.3 [M+H]⁺.

### Step 5

### Intermediate 201: tert-butyl (R)-6-(6-(1-(tert-butoxycarbonyl)piperidin-4-yl)hexanamido)-2-(1-methylpyrrolidin-2-yl)-1H-pyrrolo[3,2-c]pyridine-1-carboxylate

To a solution of tert-butyl 4-(6-amino-6-oxohexyl)piperidine-1-carboxylate (Intermediate 200, 0.20 g, 0.67 mmol) and tert-butyl (R)-6-bromo-2-(1-methylpyrrolidin-2-yl)-1H-pyrrolo[3,2-c]pyridine-1-carboxylate (Intermediate 4, 0.25 g, 0.67 mmol) in 1,4-dioxane (5 mL) was added cesium carbonate (0.66 g, 2.01 mmol). The reaction mixture was purged with argon for 20 min then Pd₂(dba)₃ (0.08 g., 0.087 mmol) and Xantphos (0.12 g, 0.20 mmol) were added the reaction was purged further with argon for 10 min before heating at 130° C. for 2 h. The reaction mixture was then concentrated in vacuo and the crude material was purified by combi-flash column chromatography, by eluting with 85% EtOAc in heptane, to afford tert-butyl (R)-6-(6-(1-(tert-butoxycarbonyl)piperidin-4-yl)hexanamido)-2-(1-methylpyrrolidin-2-yl)-1H-pyrrolo[3,2-c]pyridine-1-carboxylate (Intermediate 201, 0.31 g, 93%) as an off white solid. ¹H NMR (400 MHz, DMSO-d₆) δ ppm 0.90-0.94 (m, 2H), 1.17-1.21 (m, 2H), 1.25-1.29 (m, 5H), 1.37 (s, 9H), 1.58-1.66 (m, 6H), 1.70 (s, 9H), 1.73-1.75 (m, 2H), 2.31-2.39 (m, 5H), 2.69 (s, 3H), 3.08-3.15 (m, 1H), 3.87-3.90 (m, 3H), 6.69 (br s, 1H), 8.49 (s, 1H), 8.79 (s, 1H), 10.33 (s, 1H). Mass spec: m/z 598.3 [M+H]⁺.

### Step 6

### Intermediate 202: (R)—N-(2-(1-methylpyrrolidin-2-yl)-1H-pyrrolo[3,2-c]pyridin-6-yl)-6-(piperidin-4-yl) hexanamide dihydrochloride

To a solution of tert-butyl (R)-6-(6-(1-(tert-butoxycarbonyl)piperidin-4-yl)hexanamido)-2-(1-methylpyrrolidin-2-yl)-1H-pyrrolo[3,2-c]pyridine-1-carboxylate (Intermediate 201, 0.31 g, 0.63 mmol) in 1,4-dioxane (5 mL) was added 4M HCl in 1,4-dioxane (6 mL) at 0° C. and the reaction mixture was stirred at room temperature for 16 h. The reaction mixture was then concentrated in vacuo to obtain crude material and purified by triturating with diethyl ether and dried to afford (R)—N-(2-(1-methylpyrrolidin-2-yl)-1H-pyrrolo[3,2-c]pyridin-6-yl)-6-(piperidin-4-yl)hexanamide dihydrochloride (Intermediate 202, 0.28 g) as an off-white solid which was used for the next step without further purification. ¹H NMR (400 MHz, DMSO-d₆) δ ppm 1.17-1.37 (m, 8H), 1.62-1.78 (m, 4H), 2.31-2.47 (m, 4H) 2.70 (s, 3H), 2.73-2.89 (m, 5H), 3.18-3.28 (m, 2H) 3.56-3.58 (m, 1H), 3.60-3.78 (m, 2H), 4.62-4.63 (m, 1H), 6.96 (br s, 1H), 7.32 (s, 2H), 8.01 (s, 1H), 8.28 (s, 1H), 8.54 (br s, 1H), 8.73 (br s, 1H), 10.59 (s, 1H). Mass spec: m/z 398.2 [M−H]⁺.

### Step 7

### Example 43: 6-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)hexanamide

To a solution of (R)—N-(2-(1-methylpyrrolidin-2-yl)-1H-pyrrolo[3,2-c]pyridin-6-yl)-6-(piperidin-4-yl)hexanamide dihydrochloride (Intermediate 202, 0.28 g, 0.70 mmol) in DMSO (5 mL) was added 2-(2,6-dioxopiperidin-3-yl)-4-fluoroisoindoline-1,3-dione (CAS No: 835616-60-9, 0.20 g, 0.70 mmol) followed by N,N-diisopropylethylamine (0.46 g, 3.52 mmol) and the reaction mixture was heated at 110° C. for 6 h. The reaction mixture was cooled to room temperature, and cold water (50 mL) was added to obtain yellow precipitation, which was filtered and dried under vacuum. The crude compound was purified by preparative HPLC (Method A) to afford 6-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)hexanamide (Example 43, 0.06 g, 14%) as a yellow solid. ¹H NMR (400 MHz, DMSO-d₆) δ ppm 1.28-1.34 (m, 9H), 1.60-1.63 (m, 2H), 1.75-1.89 (m, 5H), 2.02-2.05 (m, 1H), 2.08-2.13 (m, 1H), 2.14 (s, 3H), 2.22-2.28 (m, 1H), 2.32-2.39 (m, 2H), 2.54-2.63 (m, 2H), 2.77-2.91 (m, 3H), 3.10-3.16 (m, 1H), 3.26-3.29 (m, 1H), 3.65-3.69 (m, 2H), 5.06-5.11 (m, 1H), 6.34 (s, 1H), 7.31-7.32 (m, 2H), 7.64-7.69 (m, 1H), 8.09 (s, 1H), 8.41 (s, 1H), 10.11 (br s, 1H), 11.07 (br s, 1H), 11.25 (br s, 1H). Mass spec: m/z 654.3 [M+H]⁺.

### Example 44 was Synthesised Following Scheme 42

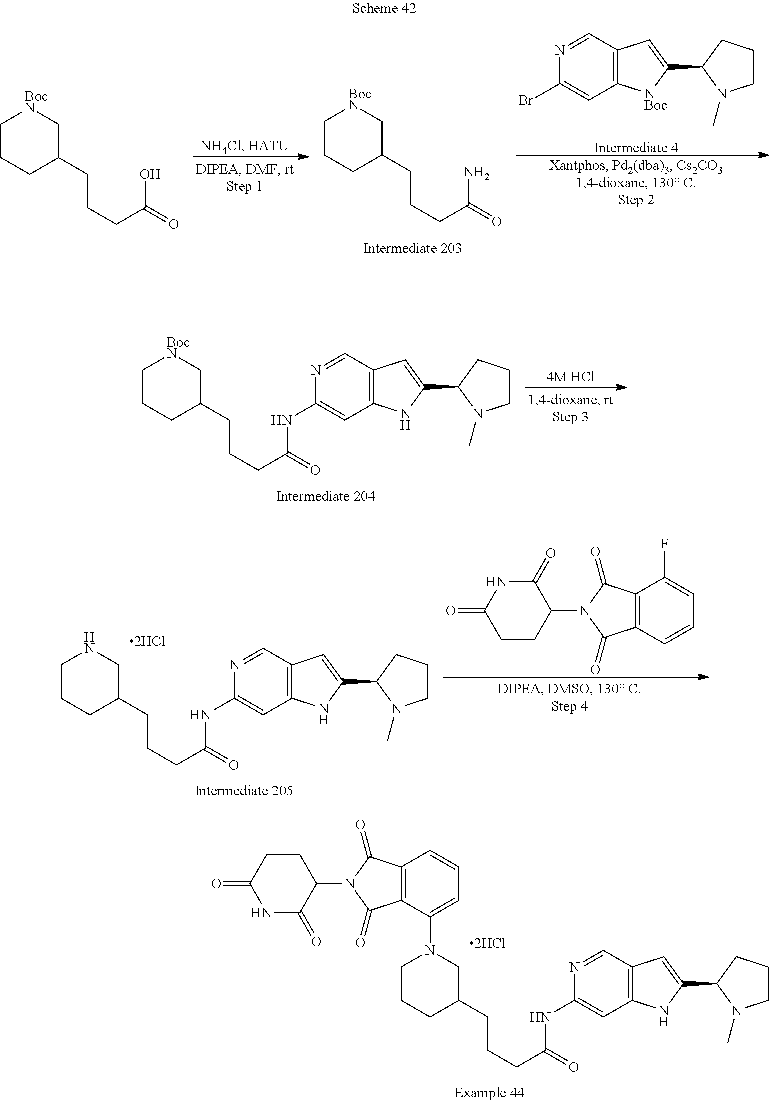

### Step 1

### Intermediate 203: tert-butyl 3-(4-amino-4-oxobutyl) piperidine-1-carboxylate

To a solution of 4-(1(tert-butoxycarbonyl)piperidin-3-yl)butanoic acid (CAS No: 318536-95-7, 2 g, 7.369 mmol) in DMF (30 mL) was added HATU (4.33 g, 11.05 mmol). The resulting reaction mixture was stirred at 15 min then added ammonium chloride (1.97 g, 36.85 mmol) and N,N-diisopropylethylamine (3.89 g, 29.48 mmol) at room temperature. The reaction mixture was stirred at room temperature for 2 h. The reaction mixture was diluted with water (200 mL) and extracted with EtOAc (2×200 mL). The organic layer was dried over Na₂SO₄ and concentrated to give crude material which was purified by combi-flash chromatography, by eluting with 45% EtOAc in heptane, to afford tert-butyl 3-(4-amino-4-oxobutyl) piperidine-1-carboxylate (Intermediate 203, 1.20 g, 60%) as a yellow solid. ¹H NMR (400 MHz, DMSO-d₆) δ ppm 1.00-1.19 (m, 3H) 1.39 (s, 9H) 1.45-1.59 (m, 3H) 1.65-1.75 (m, 1H) 2.00-2.03 (m, 2H) 2.23-2.42 (m, 1H) 2.53-2.65 (m, 2H) 2.73-2.79 (m, 1H) 3.56-3.59 (m, 2H), 6.67 (s, 1H) 7.21 (s, 1H). Mass spec: m/z 270.8 [M+H]⁺.

### Step 2

### Intermediate 204: tert-butyl 3-(4-((2-((R)-1-methylpyrrolidin-2-yl)-1H-pyrrolo[3,2-c]pyridin-6-yl)amino)-4-oxobutyl)piperidine-1-carboxylate

To a solution of tert-butyl 3-(4-amino-4-oxo-butyl)piperidine-1-carboxylate (Intermediate 203, 0.227 g, 0.84 mmol) and tert-butyl (R)-6-bromo-2-(1-methylpyrrolidin-2-yl)-1H-pyrrolo[3,2-c]pyridine-1-carboxylate (Intermediate 4, 0.40 g, 1.05 mmol) and Cesium carbonate (1.03 g, 3.15 mmol) in 1,4-dioxane (15 mL) was added. The reaction mixture was degassed with argon for 10 min, then Pd₂(dba)₃ (0.148 g, 0.15 mmol) and Xantphos (0.188 g, 0.31 mmol) was added. The resulting reaction mixture was heated at 130° C. for 2 h. The reaction mixture was then concentrated in vacuo to obtain crude compound, which was purified by combi-flash column chromatography, by eluting with 50% EtOAC in heptane to afford tert-butyl 3-(4-((2-((R)-1-methylpyrrolidin-2-yl)-1H-pyrrolo[3,2-c]pyridin-6-yl)amino)-4-oxobutyl) piperidine-1-carboxylate (Intermediate 204, 0.22 g, 44%) as an off white solid. ¹H NMR (400 MHz, DMSO-d₆) δ ppm 1.12-1.27 (m, 2H), 1.38 (s, 9H), 1.51-1.68 (m, 4H), 1.69-1.96 (m, 4H), 2.09-2.12 (m, 2H) 2.14 (s, 3H) 2.19-2.30 (m, 2H), 2.35 (t, J=7.13 Hz, 2H), 2.70-2.77 (m, 1H), 3.10-3.14 (m, 1H), 3.26-3.30 (m, 3H) 3.64-3.81 (m, 1H), 6.33 (s, 1H), 8.07 (s, 1H), 8.40 (s, 1H), 10.11 (br,s, 1H), 11.24 (s, 1H). Mass spec: m/z 470.0 [M+H]⁺.

### Step 3

### Intermediate 205: N-(2-((R)-1-methylpyrrolidin-2-yl)-1H-pyrrolo[3,2-c]pyridin-6-yl)-4-(piperidin-3-yl) butanamide dihydrochloride

To a solution of tert-butyl 3-(4-((2-((R)-1-methylpyrrolidin-2-yl)-1H-pyrrolo[3,2-c]pyridin-6-yl)amino)-4-oxobutyl) piperidine-1-carboxylate (Intermediate 204, 0.320 g, 0.32 mmol) in 1,4-dioxane (5.0 mL) was added 4M HCl in 1,4-dioxane (5.0 mL) at 0° C. The resulting reaction mixture was stirred at room temperature for 16 h. The progress of the reaction was monitored by TLC. After completion, the reaction mixture was concentrated in vacuo to afford N-(2-((R)-1-methylpyrrolidin-2-yl)-1H-pyrrolo[3,2-c]pyridin-6-yl)-4-(piperidin-3-yl) butanamide dihydrochloride (Intermediate 205, 0.30 g) as an off-white solid used for next step without further purification.

### Step 4

### Example 44: 4-(1-(2-(2,6-dioxopiperidin-3-yl)-1,3-dioxoisoindolin-4-yl)piperidin-3-yl)-N-(2-((R)-1-methylpyrrolidin-2-yl)-1H-pyrrolo[3,2-c]pyridin-6-yl)butanamide

To a solution of N-(2-((R)-1-methylpyrrolidin-2-yl)-1H-pyrrolo[3,2-c]pyridin-6-yl)-4-(piperidin-3-yl) butanamide dihydrochloride (Intermediate 205, 0.30 g, 0.81 mmol) and 2-(2,6-dioxo-3 piperidyl)-4-fluoro-isoindoline-1,3-dione (CAS No: 835616-60-9, 0.22 g, 0.81 mmol) in DMSO (1.0 mL) was added DIPEA (0.53 g, 3.0 mmol). The resulting reaction mixture was heated at 130° C. for 2 h. The reaction mixture was then poured into ice cold water, a solid precipitated which was filtered and dried to obtain crude compound. The crude compound was purified by preparative HPLC (Method A) to afford 4-(1-(2-(2,6-dioxopiperidin-3-yl)-1,3-dioxoisoindolin-4-yl)piperidin-3-yl)-N-(2-((R)-1-methylpyrrolidin-2-yl)-1H-pyrrolo[3,2-c]pyridin-6-yl)butanamide (Example 44, 0.11 g, 22%) as a yellow solid. ¹H NMR (400 MHz, DMSO-d₆) δ ppm 1.06-1.07 (m, 1H), 1.17-1.34 (m, 2H), 1.58-1.94 (m, 10H), 1.97-2.06 (m, 1H), 2.07-2.16 (m, 1H), 2.18 (s, 3H), 2.21-2.26 (m, 1H), 2.32-2.40 (m, 2H), 2.49-2.51 (m, 1H), 2.76-2.91 (m, 2H), 3.10-3.15 (m, 1H), 3.28-3.30 (m, 2H), 3.61-3.70 (m, 2H), 5.09-5.11 (m, 1H), 6.33 (s, 1H), 7.29-7.35 (m, 2H), 7.66 (t, J=7.70 Hz, 1H), 8.07 (s, 1H), 8.39 (s, 1H), 10.10 (s, 1H), 11.07 (s, 1H), 11.24 (s, 1H). Mass spec: m/z 626.3 [M+H]⁺.

### Example 45 was Synthesised Following Scheme 43

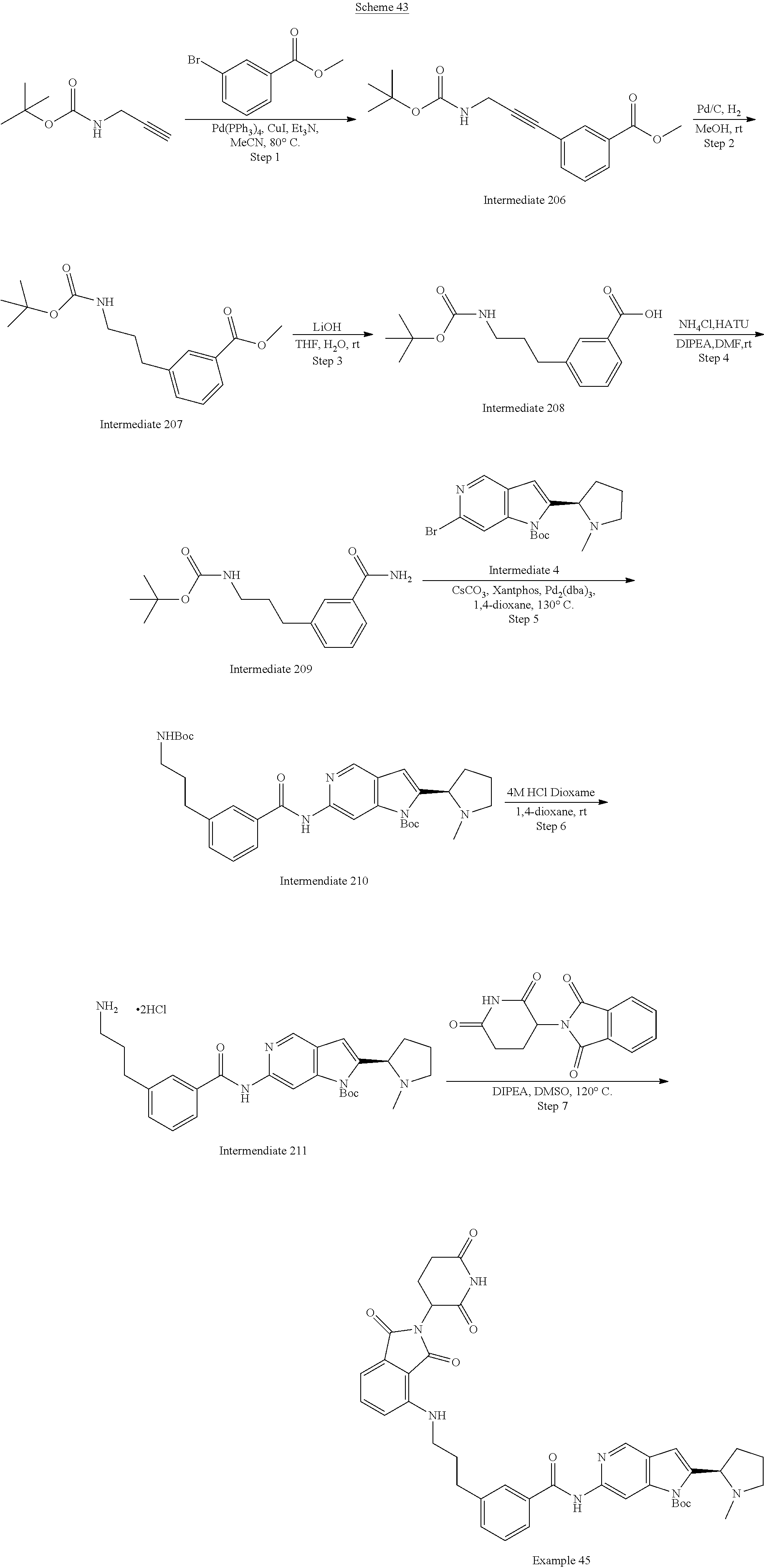

### Step 1

### Intermediate 206: methyl 3-(3-((tert-butoxycarbonyl)amino) prop-1-yn-1-yl)benzoate

To a solution of methyl 3-bromobenzoate (CAS No: 618-89-3, 4.00 g, 18.60 mmol) and tert-butyl prop-2-yn-1-ylcarbamate (CAS No: 92136-39-5, 5.76 g, 37.16 mmol) in acetonitrile (40 mL) was added Pd(PPh₃)₄ (2.20 g, 1.90 mmol) followed by copper(I) iodide (0.71 g, 3.67 mmol) and triethylamine (9 mL, 64.2 mmol) at room temperature. The reaction mixture was degassed with argon gas for 10 min then heated at 80° C. for 16 h. Progress of the reaction was monitored by TLC. After completion, the reaction mixture was extracted with EtOAc (200 mL). The organic layer was separated, dried over anhydrous Na₂SO₄ and concentrated in vacuo. The crude material was purified by combi-flash column chromatography, by eluting with 20% EtOAc in heptane, to afford methyl 3-(3-((tert-butoxycarbonyl)amino)prop-1-yn-1-yl)benzoate (Intermediate 206, 2.0 g, 37%) as a yellow solid. ¹H NMR (400 MHz, DMSO-d₆) δ ppm 1.40 (s, 9H), 3.86 (s, 3H), 3.99 (d, J=5.38 Hz, 2H), 7.37 (br s, 1H), 7.51-7.55 (m, 1H), 7.66-7.68 (m, 1H), 7.91-7.93 (m, 1H), 7.94 (s, 1H).

### Step 2

### Intermediate 207: methyl 3-(3-((tert-butoxycarbonyl)amino)propyl)benzoate

To a solution of methyl 3-(3-((tert-butoxycarbonyl)amino)prop-1-yn-1-yl)benzoate (Intermediate 206, 2.00 g, 6.91 mmol) in methanol (20 mL) was added 300 mg of 10% by wt. Pd/C at room temperature and the reaction mixture was stirred for 24 h under H₂ atmosphere at 150 psi. Progress of the reaction was monitored by TLC. After completion, the reaction mixture was passed through celite bed and concentrated in vacuo to afford methyl 3-(3-((tert-butoxycarbonyl)amino)propyl)benzoate (Intermediate 207, 1.0 g) as an off-white solid which was used for the next step without further purification. ¹H NMR (400 MHz, DMSO-d₆) δ ppm 1.37 (s, 9H), 1.65-1.69 (m, 2H), 2.61-2.65 (m, 2H), 2.91-2.93 (m, 2H), 3.84 (s, 3H), 6.87 (br s, 1H), 7.42-7.48 (m, 2H), 7.76-7.79 (m, 2H). Mass spec: m/z 193.8 [M+H−100]*.

### Step 3

### Intermediate 208: 3-(3-((tert-butoxycarbonyl)amino)propyl)benzoic acid

To a solution of methyl 3-(3-((tert-butoxycarbonyl)amino)propyl)benzoate (Intermediate 207, 0.35 g, 1.19 mmol) in tetrahydrofuran (2 mL), methanol (20 mL) was added lithium hydroxide (0.075 g, 2.38 mmol) in water (2 mL) at room temperature. The reaction mixture was stirred at room temperature for 16 h. Progress of the reaction was monitored by TLC. After completion, the reaction mixture was concentrated in vacuo. The reaction mixture was diluted with water (25 mL), acidified with 1N HCl to pH~4. The yellow solid precipitated, which was filtered and dried to obtain 3-(3-((tert-butoxycarbonyl)amino)propyl)benzoic acid (Intermediate 208, 0.21 g, 63%) as yellow solid which was used as such for the next step without further purification. ¹H NMR (400 MHz, DMSO-d₆) δ ppm 1.37 (s, 9H), 1.66-1.70 (m, 2H), 2.60-2.66 (m, 2H), 2.90-2.93 (m, 2H), 6.85 (br s, 1H), 7.37-7.46 (m, 2H), 7.74-7.80 (m, 2H), 12.86 (br s, 1H).

### Step 4

### Intermediate 209: tert-butyl (3-(3-carbamoylphenyl)propyl)carbamate

To a solution of 3-(3-((tert-butoxycarbonyl)amino)propyl)benzoic acid (Intermediate 208, 0.20 g, 0.72 mmol) in DMF (3 mL) was added HATU (0.42 g, 1.07 mmol) and diisopropylethylamine (0.27 g, 2.14 mmol) at RT. The reaction mixture was stirred at room temperature for 15 min then added ammonium chloride (0.15 g, 2.86 mmol). Reaction mixture was stirred at RT for 16 h. Progress of the reaction was monitored by TLC. After completion, the reaction mixture was diluted with water (100 mL) and extracted with EtOAc (3×50 mL). The organic layer was washed with NaHCO₃ (2×40 mL) and brine solution (2×30 mL). The organic layer was separated, dried over Na₂SO₄ and concentrated in vacuo to obtain crude compound. The crude material was purified by combi-flash column chromatography by eluting with 15% EtOAc in heptane to afford tert-butyl (3-(3-carbamoylphenyl)propyl)carbamate (Intermediate 209, 0.15 g, 75%) as a colorless liquid. ¹H NMR (400 MHz, DMSO-d₆) δ ppm 1.26 (s, 9H), 2.68-2.73 (m, 2H), 3.12-3.15 (m, 2H), 3.59-3.64 (m, 2H) 6.86 (br s, 1H), 7.19-7.34 (m, 2H), 7.67-7.70 (m, 1H), 7.90-7.95 (m, 1H), 8.18 (br s, 2H).

### Step 5

### Intermediate 210: tert-butyl (R)-6-(3-(3-((tert-butoxycarbonyl)amino)propyl)benzamido)-2-(1-methylpyrrolidin-2-yl)-1H-pyrrolo[3,2-c]pyridine-1-carboxylate

To a solution of tert-butyl (3-(3-carbamoylphenyl)propyl)carbamate (Intermediate 209, 0.30 g, 1.07 mmol) in 1,4-dioxane (5 mL) was added tert-butyl (R)-6-bromo-2-(1-methylpyrrolidin-2-yl)-1H-pyrrolo[3,2-c]pyridine-1-carboxylate (Intermediate 4, 0.14 g, 1.29 mmol) followed by cesium carbonate (1.06 g, 3.23 mmol). The reaction mixture was purged with argon for 20 min. To this reaction mixture Pd₂(dba)₃ (0.10 g, 0.11 mmol) and xantphos (0.31 g, 0.32 mmol) were added at room temperature and purged with argon for 10 min. The reaction mixture was heated at 130° C. for 1 h. Progress of the reaction was monitored by TLC. After completion, the reaction mixture diluted with water (50 mL) and extracted with EtOAc (3×50 mL). The organic layer was separated, dried over Na₂SO₄ and concentrated in vacuo to obtain crude compound. The crude material was purified by combi-flash column chromatography by eluting with 70% EtOAc in heptane to afford tert-butyl (R)-6-(3-(3-((tert-butoxycarbonyl)amino)propyl)benzamido)-2-(1-methylpyrrolidin-2-yl)-1H-pyrrolo[3,2-c]pyridine-1-carboxylate (Intermediate 210, 0.30 g, 51%) as an off-white solid. ¹H NMR (400 MHz, DMSO-d₆) δ ppm 1.38 (s, 9H), 1.69 (s, 9H), 1.71-1.79 (m, 5H), 2.35 (s, 3H), 2.36-2.39 (m, 2H), 2.64 (t, J=7.45 Hz, 2H), 2.93-2.96 (m, 2H), 3.10-3.12 (m, 1H), 3.89-3.95 (m, 1H), 6.75 (s, 1H), 6.87 (s, 1H), 7.39-7.41 (m, 2H), 7.84-7.89 (m, 2H), 8.59 (s, 1H), 8.92 (s, 1H), 10.63 (s, 1H). Mass spec: m/z 576.2 [M−H]⁻.

### Step 6

### Intermediate 211: (R)-3-(3-aminopropyl)-N-(2-(1-methylpyrrolidin-2-yl)-1H-pyrrolo[3,2-c]pyridin-6-yl)benzamine dihydrochloride

To a solution of tert-butyl (R)-6-(3-(3-((tert-butoxycarbonyl)amino)propyl)benzamido)-2-(1-methylpyrrolidin-2-yl)-1H-pyrrolo[3,2-c]pyridine-1-carboxylate (Intermediate 210, 0.30 g, 0.546 mmol) in 1,4-dioxane (2.5 mL) was added 4M HCl in 1,4-dioxane (5 mL) at 0° C. The reaction mixture was stirred at room temperature for 16 h. Progress of the reaction was monitored by TLC. After completion, the reaction mixture was concentrated in vacuo, washed with DCM (2×20 mL) and dried. The crude material was washed with diethyl ether (2×20 mL) and dried to afford (R)-3-(3-aminopropyl)-N-(2-(1-methylpyrrolidin-2-yl)-1H-pyrrolo[3,2-c]pyridin-6-yl)benzamide dihydrochloride (Intermediate 211, 0.22 g crude) as an off white solid which was used for next step without further purification. ¹H NMR (400 MHz, DMSO-d₆) δ ppm 1.93-2.00 (m, 2H), 2.15-2.22 (m, 2H), 2.37-2.45 (m, 2H), 2.53-2.56 (m, 1H), 2.73 (s, 3H), 2.75-2.84 (m, 3H), 3.16-3.25 (m, 1H), 3.73-3.75 (m, 1H), 4.72-4.74 (m, 1H), 7.26 (s, 1H), 7.53-7.57 (m, 2H), 8.02 (s, 3H), 8.10 (s, 1H), 8.31 (s, 1H), 9.08 (s, 1H), 11.47 (br s, 1H), 11.98 (br s, 1H), 13.21 (br s, 1H).

### Step 7

### Example 45: 3-(3-((2-(2,6-dioxopiperidin-3-yl)-1,3-dioxoisoindolin-4-yl)amino)propyl)-N-(2-((R)-1-methylpyrrolidin-2-yl)-1H-pyrrolo[3,2-c]pyridin-6-yl)benzamide

To a solution of (R)-3-(3-aminopropyl)-N-(2-(1-methylpyrrolidin-2-yl)-1H-pyrrolo[3,2-c]pyridin-6-yl)benzamide dihydrochhloride (Intermediate 211, 0.24 g, 0.63 mmol) in DMSO (5 mL) was added 2-(2,6-dioxo-3 piperidyl)-4-fluoro-isoindoline-1,3-dione (CAS No: 835616-60-9, 0.17 g, 0.63 mmol) followed by diisopropylethylamine (0.33 mL 1.91 mmol) at room temperature. The reaction mixture was purged with argon for 15 min. The reaction mixture was heated at 100° C. for 16 h. Progress of the reaction was monitored by TLC. After completion, the reaction mixture was cooled to room temperature, treated with cold water (50 mL), filtered, dried, and washed with DCM/pentane (2×20 mL) and then dried. The crude material was purified by preparative HPLC (Method A) to afford 3-(3-((2-(2,6-dioxopiperidin-3-yl)-1,3-dioxoisoindolin-4-yl)amino)propyl)-N-(2-((R)-1-methylpyrrolidin-2-yl)-1H-pyrrolo[3,2-c]pyridin-6-yl)benzamide (Example 45, 0.8 g, 19.9%) as a yellow solid. ¹H NMR (400 MHz, DMSO-d₆) δ ppm 1.86-2.16 (m, 7H), 2.16-2.24 (m, 1H), 2.25 (s, 3H), 2.47-2.49 (m, 1H), 2.53-2.60 (m, 2H), 2.74-2.78 (m, 2H), 2.83-2.92 (m, 1H), 3.12-3.16 (m, 1H), 3.34-3.39 (m, 2H), 5.03-5.07 (m, 1H), 6.39 (s, 1H), 6.64 (t, J=5.87 Hz, 1H), 7.02 (d, J=6.85 Hz, 1H), 7.09 (d, J=8.56 Hz, 1H), 7.38-7.44 (m, 2H), 7.56-7.60 (m, 1H), 7.83-7.86 (m, 1H), 7.94 (s, 1H), 8.19 (s, 1H), 8.49-8.50 (m, 1H), 10.42 (br s, 1H), 11.37 (br s, 1H). Mass spec: m/z 634.4 [M+H].

### Example 46 was Synthesised Following Scheme 44

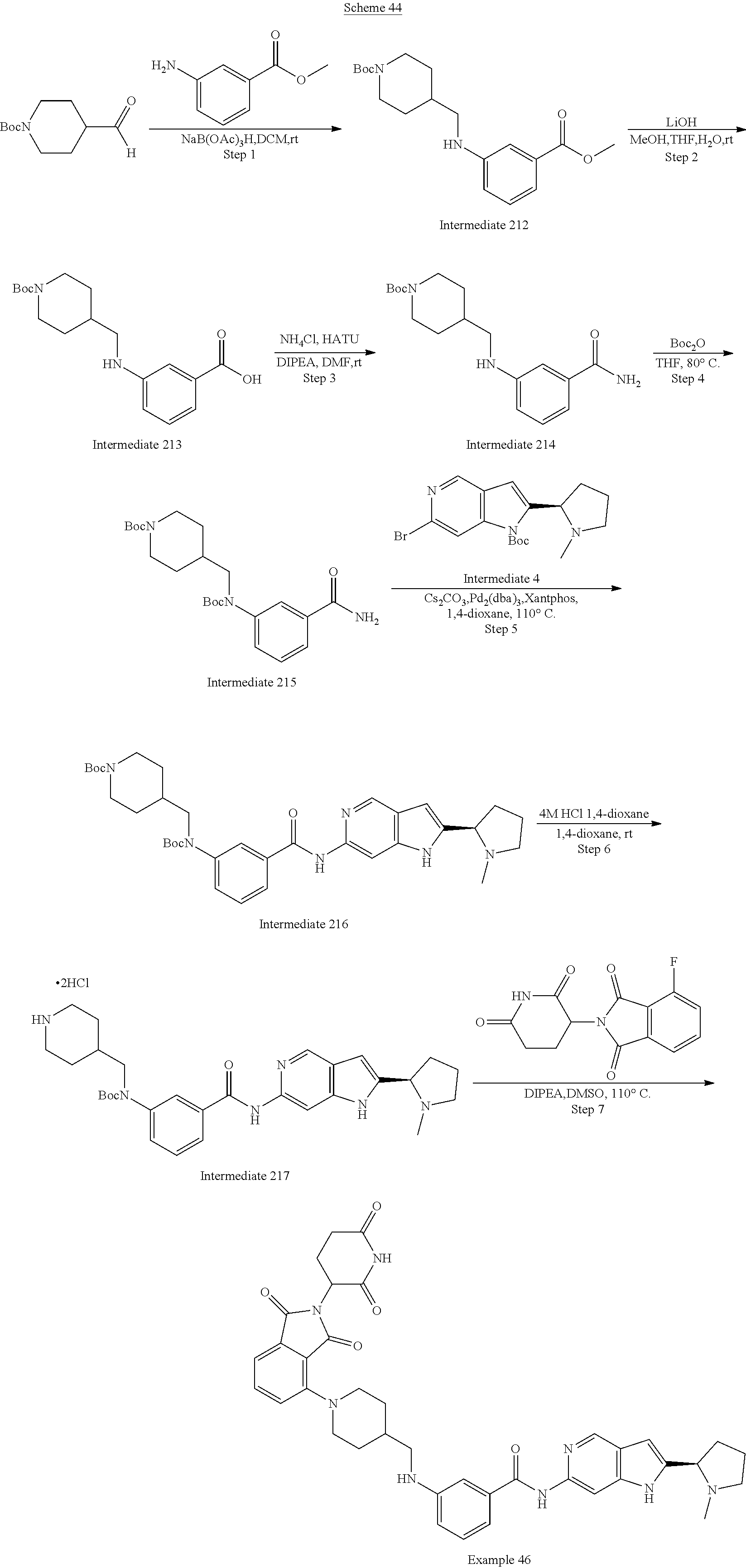

### Step 1

### Intermediate 212: tert-butyl 4-(((3-(methoxycarbonyl)phenyl)amino)methyl)piperidine-1-carboxylate

To a solution of tert-butyl 4-formylpiperidine-1-carboxylate (CAS No: 137076-22-3, 5.0 g, 33.07 mmol) in dichloromethane (100 mL) was added methyl 3-aminobenzoate (CAS No: 4518-10-9, 7.75 g, 36.38 mmol). The reaction mixture was stirred at room temperature for 2 h and then added sodium triacetoxyborohydride (21.68 g, 99.23 mmol) at room temperature and the reaction mixture was stirred at rt for 16 h. Progress of the reaction was monitored by TLC. After completion, the reaction mixture was diluted with water (100 mL) and extracted with DCM (2×200 mL). The organic layer was separated, dried over anhydrous Na₂SO₄ and concentrated in vacuo. The crude material was purified by combi-flash column chromatography by eluting with 60% EtOAc in heptane to afford tert-butyl 4-(((3-(methoxycarbonyl)phenyl)amino)methyl)piperidine-1-carboxylate (Intermediate 212, 10.0 g, 87%) as a yellow solid. ¹H NMR (400 MHz, DMSO-d₆) δ ppm 1.01-1.09 (m, 2H), 1.39 (s, 9H), 1.71-1.74 (m, 3H), 2.64-2.67 (m, 2H), 2.91-2.94 (m, 2H), 3.80 (s, 3H), 3.93-3.96 (m, 2H), 5.99 (br s, 1H), 6.81 (d, J=7.89 Hz, 1H), 7.09 (d, J=7.20 Hz, 1H), 7.13-7.15 (m, 1H), 7.18 (s, 1H). Mass spec: m/z 347.1 [M+H]⁺.

### Step 2

### Intermediate 213: 3-(((1-(tert-butoxycarbonyl)piperidin-4-yl)methyl)amino)benzoic acid

To a solution of tert-butyl 4-(((3-(methoxycarbonyl)phenyl)amino)methyl)piperidine-1-carboxylate (Intermediate 212, 3.0 g, 8.61 mmol) in tetrahydrofuran (5 mL) and methanol (5 mL) was added lithium hydroxide (0.631 g, 25.8 mmol) in water (10 mL) at 0° C. The reaction mixture was stirred at room temperature for 6 h. Progress of the reaction was monitored by TLC. After completion, the reaction mixture was concentrated in vacuo. The reaction was diluted with water (20 mL) and the pH of solution was adjusted to 3-4 with 0.5 N HCl and extracted with EtOAc (2×100 mL). The organic layer was dried over Na₂SO₄ and concentrated in vacuo. to afford 3-(((1-(tert-butoxycarbonyl)piperidin-4-yl)methyl)amino)benzoic acid (Intermediate 213, 2.6 g, 93%) as a sticky liquid. ¹H NMR (400 MHz, DMSO-d₆) δ ppm 1.39 (s, 9H), 1.73-1.76 (m, 4H), 2.67-2.69 (m, 2H), 2.90-2.94 (m, 2H), 3.37-3.40 (m, 2H), 3.93-3.95 (m, 1H), 5.91 (br s, 1H), 6.78 (d, J=8.11 Hz, 1H), 7.07-7.09 (m, 1H), 7.13-7.15 (m, 1H), 7.17 (s, 1H). Mass spec: m/z 235.1 [M+H]⁺.

### Step 3

### Intermediate 214: tert-butyl 4-(((3-carbamoylphenyl)amino)methyl)piperidine-1-carboxylate

To a solution of 3-(((1-(tert-butoxycarbonyl)piperidin-4-yl)methyl)amino)benzoic acid (Intermediate 213, 3.8 g, 11.0 mmol) in DMF (25 mL) was added diisopropylethylamine (6.0 mL, 34.0 mmol) and HATU (6.7 g, 17.0 mmol) followed by ammonium chloride (2.4 g, 45.0 mmol). The reaction mixture was stirred at room temperature for 16 h. Progress of the reaction was monitored by TLC. After completion, the reaction mixture was diluted with water (200 mL) and extracted with EtOAc (3×100 mL). The organic layer was separated, dried over anhydrous Na₂SO₄ and concentrated in vacuo to afford tert-butyl 4-(((3-carbamoylphenyl)amino)methyl)piperidine-1-carboxylate (Intermediate 214, 3.4 g) as a yellow solid, which was used for the next step without further purification. ¹H NMR (400 MHz, DMSO-d₆) δ ppm 1.02-1.05 (m, 2H), 1.39 (s, 9H), 1.71-1.74 (m, 3H), 2.69-2.73 (m, 2H), 2.89-2.94 (m, 2H), 3.91-3.94 (m, 2H), 5.81 (br s, 1H), 6.69 (d, J=7.89 Hz, 1H), 6.93-7.04 (m, 2H), 7.07-7.11 (m, 1H), 7.16 (br s, 1H), 7.75 (br s, 1H). Mass spec: m/z 333.7 [M]⁺.

### Step 4

### Intermediate 215: tert-butyl 4-(((tert-butoxycarbonyl)(3-carbamoylphenyl)amino)methyl)piperidine-1-carboxylate

To a solution of tert-butyl 4-(((3-carbamoylphenyl)amino)methyl)piperidine-1-carboxylate (Intermediate 214, 3.2 g, 9.6 mmol) in tetrahydrofuran (30 mL) was added di-tert-butyl dicarbonate (6.3 g, 29.0 mmol). The reaction mixture was heated at 80° C. for 16 h. Progress of the reaction was monitored by TLC. After completion, the reaction mixture was concentrated in vacuo. The crude material was purified by combi-flash column chromatography by eluting with 95% EtOAc in heptane to afford tert-butyl 4-(((tert-butoxycarbonyl)(3-carbamoylphenyl)amino)methyl)piperidine-1-carboxylate (Intermediate 215, 3.5 g, 84%) as a sticky liquid material. ¹H NMR (500 MHz, DMSO-d₆) δ ppm 1.36 (s, 9H), 1.37 (s, 9H), 1.53-1.59 (m, 4H), 2.61-2.63 (m, 1H), 3.16-3.18 (m, 1H), 3.55-3.57 (m, 2H), 3.85-3.88 (m, 2H), 4.00-4.05 (m, 1H), 7.38-7.45 (m, 3H), 7.70-7.72 (m, 1H), 7.73 (br s, 1H), 8.00 (br s, 1H). Mass spec: m/z 433.8 [M]⁺.

### Step 5

### Intermediate 216: tert-butyl (R)-6-(3-((tert-butoxycarbonyl)((1-(tert-butoxycarbonyl)piperidin-4-yl)methyl)amino)benzamido)-2-(1-methylpyrrolidin-2-yl)-1H-pyrrolo[3,2-c]pyridine-1-carboxylate

To a solution of tert-butyl 4-(((tert-butoxycarbonyl)(3-carbamoylphenyl)amino)methyl)piperidine-1-carboxylate (Intermediate 215, 0.4 g, 0.92 mmol) in 1,4-dioxane (6 mL) was added tert-butyl (R)-6-bromo-2-(1-methylpyrrolidin-2-yl)-1H-pyrrolo[3,2-c]pyridine-1-carboxylate (Intermediate 4, 0.38 g, 1.01 mmol) followed by caesium carbonate (0.90 g, 2.76 mmol). The reaction mixture was purged with argon for 20 min. To the reaction mixture Pd₂(dba)₃ (0.11 g, 0.12 mmol) and xantphos (0.16 g, 0.27 mmol) were added. The reaction mixture was purged with argon for 10 min and heated at 100° C. for 2 h. Progress of the reaction was monitored by TLC. After completion, the reaction mixture was passed through a celite pad and concentrated in vacuo. The crude material was purified by combi-flash column chromatography by eluting with 85% EtOAc in heptane to afford tert-butyl (R)-6-(3-((tert-butoxycarbonyl)((1-(tert-butoxycarbonyl)piperidin-4-yl)methyl)amino)benzamido)-2-(1-methylpyrrolidin-2-yl)-1H-pyrrolo[3,2-c]pyridine-1-carboxylate (Intermediate 216, 0.4 g, 85%) as an off white solid. ¹H NMR (400 MHz, DMSO-d₆) δ ppm 1.37 (s, 9H), 1.38 (s, 9H), 1.59-1.62 (m, 6H), 1.69 (s, 9H), 1.73-1.75 (m, 2H), 2.35 (s, 3H), 2.37-2.42 (m, 1H), 2.62-2.67 (m, 2H), 3.11-3.13 (m, 1H), 3.37-3.39 (m, 1H), 3.62-3.64 (m, 2H), 3.87-3.92 (m, 3H), 6.76 (s, 1H), 7.45-3.47 (m, 2H), 7.85-7.87 (m, 1H), 7.95 (s, 1H), 8.60 (s, 1H), 8.93 (s, 1H), 10.79 (s, 1H). Mass spec: m/z 733.1 [M+H]⁺.

### Step-6

### Intermediate 217: (R)—N-(2-(1-methylpyrrolidin-2-yl)-1H-pyrrolo[3,2-c]pyridin-6-yl)-3-((piperidin-4-ylmethyl)amino) benzamide dihydrochloride

To a solution of tert-butyl (R)-6-(3-((tert-butoxycarbonyl)((1-(tert-butoxycarbonyl)piperidin-4-yl)methyl)amino)benzamido)-2-(1-methylpyrrolidin-2-yl)-1H-pyrrolo[3,2-c]pyridine-1-carboxylate (Intermediate 216, 0.50 g, 0.79 mmol) in 1,4-dioxane (10 mL) was added 4M hydrochloric acid in 1,4-dioxane (12 mL) was added at 0° C. The reaction mixture was stirred at room temperature for 16 h. Progress of the reaction was monitored by TLC. After completion, the reaction mixture was concentrated in vacuo to afford (R)—N-(2-(1-methylpyrrolidin-2-yl)-1H-pyrrolo[3,2-c]pyridin-6-yl)-3-((piperidin-4-ylmethyl)amino)benzamide dihydrochloride (Intermediate 217, 0.34 g) as a yellow solid which was used for the next step without further purification. ¹H NMR (400 MHz, DMSO-d₆) δ ppm 1.32-1.39 (m, 2H), 1.91-1.95 (m, 3H), 2.17-2.21 (m, 2H), 2.40-2.43 (m, 2H), 2.53-2.57 (m, 1H), 2.81 (s, 3H), 2.86-2.89 (m, 1H), 3.05-3.08 (m, 2H), 3.26-3.29 (m, 3H), 3.73-3.75 (m, 1H), 4.72-4.74 (m, 1H), 6.88-6.91 (m, 1H), 7.26-7.31 (m, 4H), 8.23 (s, 1H), 8.53-8.55 (m, 1H), 8.78 (br s, 1H), 9.07 (s, 1H), 11.28 (br s, 1H), 11.75 (br s, 1H), 13.18 (br s, 1H). Mass spec: m/z 432.6 [M+H]⁺.

### Step 7

### Example 46: 3-(((1-(2-(2,6-dioxopiperidin-3-yl)-1,3-dioxoisoindolin-4-yl)piperidin-4-yl)methyl)amino)-N-(2-((R)-1-methylpyrrolidin-2-yl)-1H-pyrrolo[3,2-c]pyridin-6-yl)benzamide

To a solution of (R)—N-(2-(1-methylpyrrolidin-2-yl)-1H-pyrrolo[3,2-c]pyridin-6-yl)-3-((piperidin-4-ylmethyl)amino)benzamide dihydrochloride (Intermediate 217, 0.3 g, 0.90 mmol) in dimethyl sulfoxide (5 mL) was added 2-(2,6-dioxopiperidin-3-yl)-4-fluoroisoindoline-1,3-dione (CAS No: 835616-60-9, 0.26 g, 0.90 mmol) followed by N,N-diisopropylethylamine (0.78 mL, 4.50 mmol). The reaction mixture was heated at 110° C. for 6 h. Progress of the reaction was monitored by TLC. After completion, the reaction mixture was poured in ice cold water (50 mL) to obtain yellow solid, which was filtered and dried. The crude material was purified by preparative HPLC (Method A) to afford 3-(((1-(2-(2,6-dioxopiperidin-3-yl)-1,3-dioxoisoindolin-4-yl)piperidin-4-yl)methyl)amino)-N-(2-((R)-1-methylpyrrolidin-2-yl)-1H-pyrrolo[3,2-c]pyridin-6-yl)benzamide (Example 46, 0.1 g, 19%) as a yellow solid. ¹H NMR (400 MHz, DMSO-d₆) δ ppm 1.42-1.50 (m, 2H), 1.78-1.92 (m, 6H), 1.99-2.05 (m, 1H), 2.11-2.14 (m, 1H), 2.17 (s, 3H), 2.23-2.29 (m, 1H), 2.54-2.61 (m, 2H), 2.83-2.92 (m, 3H), 3.06-3.09 (m, 2H), 3.12-3.16 (m, 1H), 3.32-3.34 (m, 1H), 3.72-3.76 (m, 2H), 5.07-5.11 (m, 1H), 5.91-5.92 (m, 1H), 6.38 (s, 1H), 6.78-6.81 (m, 1H), 7.15-7.22 (m, 3H), 7.32 (d, J=7.20 Hz, 1H), 7.35 (d, J=8.40 Hz, 1H), 7.65-7.70 (m, 1H), 8.17 (s, 1H), 8.48 (s, 1H), 10.13 (s, 1H), 11.07 (s, 1H), 11.34 (s, 1H). Mass spec: m/z: 688.0 [M+H]⁺.

### Example 47 was Synthesised Following Scheme 45

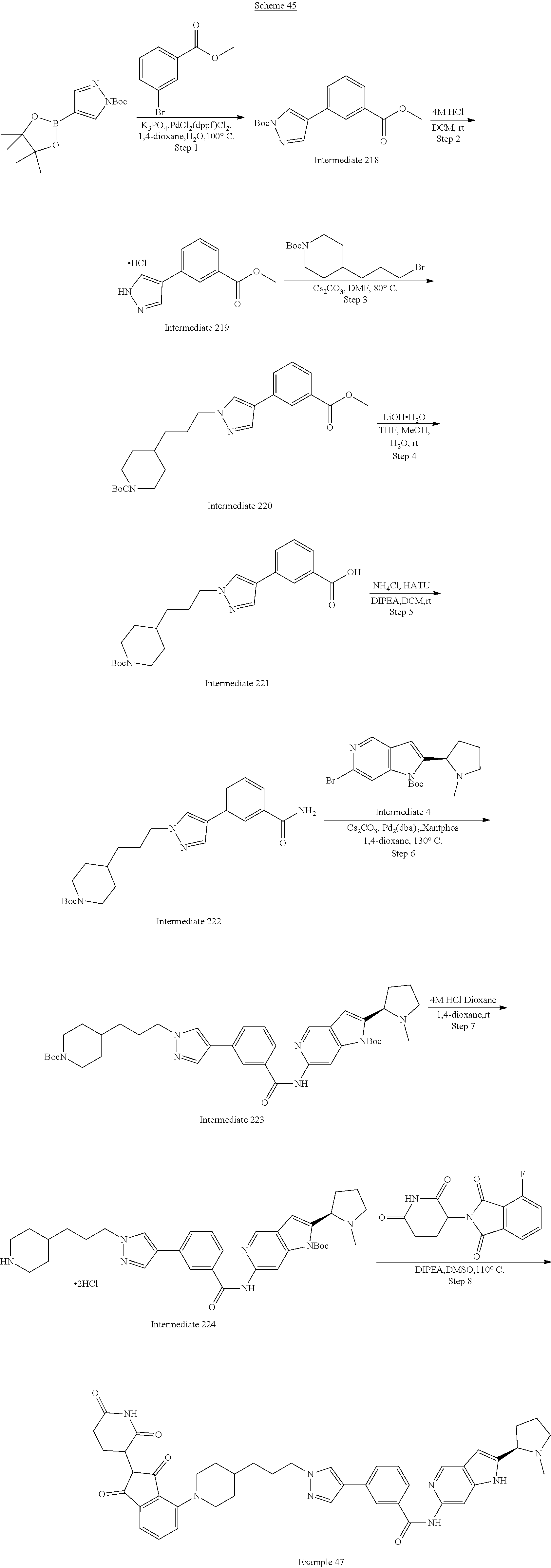

### Step 1

### Intermediate 218: tert-butyl 4-(3-(methoxycarbonyl)phenyl)-1H-pyrazole-1-carboxylate

To a solution of methyl-3-bromobenzoate (CAS No: 618-89-3, 2.5 g, 12 mmol) and tert-butyl-4-(4,4,5,5-tetramethyl-1,3,2-dioxaborolan-2-yl)pyrazole-1-carboxylate (CAS No: 552846-17-0, 3.8 g, 13 mmol) in 1,4-dioxane (10 mL) was added K₃PO₄ (6.2 g, 29 mmol) and water (2.0 mL) at room temperature. The reaction mixture was purged with argon for 10 min then Pd(dppf)Cl₂ (0.42 g, 0.58 mmol) was added, and the reaction mixture was further purged with argon for 5 min. The reaction mixture was then heated at 100° C. for 16 h. The reaction mixture was filtered through celite, water (50 mL) was added extracted with EtOAc (100 mL). The organic layer was dried over Na₂SO₄ and concentrated in vacuo. The crude material was purified by combi-flash column chromatography, eluting with 26% EtOAc in heptane, to afford tert-butyl 4-(3-(methoxycarbonyl)phenyl)-1H-pyrazole-1-carboxylate (Intermediate 218, 2.0 g, 57%) as an off white solid. ¹H NMR (400 MHz, DMSO-d₆) δ ppm 1.61 (s, 9H), 3.89 (s, 3H), 7.57-7.58 (m, 1H), 7.88 (d, J=7.89 Hz, 1H), 8.05 (d, J=8.11 Hz, 1H), 8.27 (s, 1H), 8.39 (s, 1H), 8.86 (s, 1H). Mass spec: m/z: 303.2 [M+H]⁺.

### Step 2

### Intermediate 219: methyl-3-(1H-pyrazol-4-yl)benzoate hydrochloride

To a solution of tert-butyl 4-(3-(methoxycarbonyl)phenyl)-1H-pyrazole-1-carboxylate (Intermediate 218, 2 g, 6.61 mmol) in dichloromethane (20 mL) was added 4M hydrochloric acid in 1,4-dioxane (10 mL) at 0° C. under a N₂ atmosphere and the reaction mixture was stirred at room temperature for 16 h. The reaction mixture was concentrated in vacuo, washed with EtOAc (100 mL) and pentane (100 mL) and dried in vacuo to afford methyl 3-(1H-pyrazol-4-yl)benzoate hydrochloride (Intermediate 219, 2.0 g) as a white solid, which was used in the next step without further purification. ¹H NMR (500 MHz, DMSO-d₆) δ ppm 3.87 (s, 3H), 7.51-7.52 (m, 1H), 7.76-7.78 (m, 1H), 7.88-7.91 (m, 1H), 8.14 (d, J=1.60 Hz, 1H), 8.16 (s, 1H), 8.18 (s, 1H). Mass spec: m/z: 203.4 [M+H]⁺.

### Step 3

### Intermediate 220: tert-butyl 4-(3-(4-(3-(methoxycarbonyl)phenyl)-1H-pyrazol-1-yl)propyl)piperidine-1-carboxylate

To a suspension of methyl-3-(1H-pyrazol-4-yl)benzoate hydrochloride (Intermediate 219, 2 g, 8.37 mmol) and tert-butyl 4-(3-bromopropyl)piperidine-1-carboxylate (CAS No: 164149-27-3, 2.56 g, 8.37 mmol) in DMF (10 mL) at 0° C. under N₂ atmosphere was added cesium carbonate (6.82 g, 20.95 mmol) and the reaction mixture was heated at 80° C. for 16 h. The reaction mixture was poured into ice cold water (30 mL) and extracted with EtOAc (2×200 mL). The combined organic phases were dried over Na₂SO₄ and concentrated in vacuo. The crude material was purified by combi-flash column chromatography, eluting with 10% EtOAc in heptane, to afford tert-butyl 4-(3-(4-(3-(methoxycarbonyl)phenyl)-1H-pyrazol-1-yl)propyl)piperidine-1-carboxylate (Intermediate 220, 2.5 g, 70%) as yellow solid. Mass spec: m/z: 428.3 [M+H]⁺.

### Step 4

### Intermediate 221: 3-(1-(3-(1-(tert-butoxycarbonyl)piperidin-4-yl) propyl)-1H-pyrazol-4-yl)benzoic acid

To a solution of tert-butyl 4-(3-(4-(3-(methoxycarbonyl)phenyl)-1H-pyrazol-1-yl)propyl)piperidine-1-carboxylate (Intermediate 220, 2.00 g, 4.67 mmol) in tetrahydrofuran (10 mL), methanol (10 mL) and water (5 mL) was added LiOH·H₂O (0.98 g, 23.39 mmol) and the reaction mixture was stirred at rt for 4 h. The reaction mixture was then concentrated in vacuo, water (10 mL) was added and the mixture acidified to pH~2 using aqueous citric acid. The resulting precipitate was filtered and dried under vacuum to afford 3-(1-(3-(1-(tert-butoxycarbonyl)piperidin-4-yl) propyl)-1H-pyrazol-4-yl)benzoic acid (Intermediate 221, 1.5 g, 78%) as an off white solid. ¹H NMR (400 MHz, DMSO-d₆) δ ppm 0.87-0.97 (m, 2H), 1.15-1.20 (m, 2H), 1.37 (s, 9H), 1.59-1.63 (m, 2H), 1.81-1.87 (m, 2H), 2.62-2.76 (m, 2H), 3.88-3.91 (m, 2H), 4.00-4.08 (m, 1H), 4.09-4.12 (m, 2H), 7.47 (t, J=7.75 Hz, 1H), 7.74-7.76 (m, 1H), 7.79-7.82 (m, 1H), 7.92 (s, 1H), 8.09 (s, 1H), 8.30 (s, 1H), 12.20 (br s, 1H). Mass spec: m/z: 414.2 [M+H]⁺.

### Step 5

### Intermediate 222: tert-butyl 4-(3-(4-(3-carbamoylphenyl)-1H-pyrazol-1-yl)propyl)piperidine-1-carboxylate

To a solution of 3-(1-(3-(1-(tert-butoxycarbonyl)piperidin-4-yl)propyl)-1H-pyrazol-4-yl)benzoic acid (Intermediate 221, 2.5 g, 6.0 mmol) in DCM (25 mL) was added diisopropylethylamine (2.4 g, 18 mmol) and HATU (2.8 g, 7.3 mmol) followed by ammonium chloride (1.6 g, 30 mmol) and the reaction mixture was stirred at rt for 16 h. The reaction mixture was diluted with water (200 mL) and extracted with EtOAc (2×200 mL). The combined organic phases were washed with NaHCO₃ (2×40 mL) and brine (2×30 mL). The organic layer was dried over anhydrous Na₂SO₄ and concentrated and the crude material was purified by combi-flash column chromatography, by eluting with 12% EtOAc in heptane, to afford tert-butyl 4-(3-(4-(3-carbamoylphenyl)-1H-pyrazol-1-yl)propyl)piperidine-1-carboxylate (Intermediate 222, 1.5 g, 60%) as an off-white solid. ¹H NMR (400 MHz, DMSO-d₆) δ ppm 0.83-0.97 (m, 1H), 1.37 (s, 9H), 1.52-1.59 (m, 2H), 1.79-1.86 (m, 2H), 2.66-2.73 (m, 2H), 3.08-3.17 (m, 2H), 3.57-3.64 (m, 2H), 3.88-4.01 (m, 2H), 4.08-4.12 (m, 2H), 7.47 (t, J=7.78 Hz, 1H), 7.75 (d, J=7.67 Hz, 1H), 7.81 (d, J=7.89 Hz, 1H), 7.92 (s, 1H), 8.09 (s, 1H), 8.30 (s, 1H). Mass spec: m/z: 413.3 [M+H]⁺.

### Step 6

### Intermediate 223: tert-butyl (R)-6-(3-(1-(3-(1-(tert-butoxycarbonyl) piperidin-4-yl) propyl)-1H-pyrazol-4-yl)benzamido)-2-(1-methylpyrrolidin-2-yl)-1H-pyrrolo[3,2-c]pyridine-1-carboxylate

To a solution of tert-butyl (R)-6-bromo-2-(1-methylpyrrolidin-2-yl)-1H-pyrrolo[3,2-c]pyridine-1-carboxylate (Intermediate 4, 0.400 g, 1.05 mmol) and tert-butyl 4-(3-(4-(3-carbamoylphenyl)-1H-pyrazol-1-yl)propyl)piperidine-1-carboxylate (Intermediate 222, 0.347 g, 0.84 mmol) in 1,4-dioxane (10 mL) was added cesium carbonate (1.03 g, 3.15 mmol). The reaction mixture was purged with argon for 10 min then tris(dibenzylideneacetone)dipalladium (0.148 g, 0.15 mmol) and Xantphos (0.188 g, 0.31 mmol) were added. The reaction mixture was further degassed for 5 minutes with argon and then heated at 130° C. for 2 h. The reaction mixture was diluted with water (300 mL) and extracted with EtOAc (3×300 mL). The combined organic phases were dried over anhydrous Na₂SO₄ and concentrated in vacuo. The crude material was purified by combi-flash column chromatography, eluting with 70% EtOAc in heptane, to afford tert-butyl (R)-6-(3-(1-(3-(1-(tert-butoxycarbonyl)piperidin-4-yl)propyl)-1H-pyrazol-4-yl)benzamido)-2-(1-methylpyrrolidin-2-yl)-1H-pyrrolo[3,2-c]pyridine-1-carboxylate (Intermediate 223, 0.28 g, 30%) as a yellow solid. ¹H NMR (400 MHz, DMSO-d₆) δ ppm 1.15-1.22 (m, 2H), 1.17-1.20 (m, 2H) 1.37 (s, 9H), 1.60-1.65 (m, 5H), 1.70 (s, 9H), 1.74-1.86 (m, 2H), 2.31 (s, 3H) 2.32-2.36 (m, 4H), 2.62-2.66 (m, 2H), 3.10-3.13 (m, 2H), 3.92-3.94 (m, 1H), 4.10-4.14 (m, 2H), 6.76 (s, 1H), 7.46-7.50 (m, 1H), 7.75-7.85 (m, 2H), 8.00 (s, 1H), 8.30-8.32 (m, 2H), 8.61 (s, 1H), 8.96 (s, 1H), 10.74 (s, 1H). Mass spec: m/z: 712.4 [M+H]⁺.

### Step 7

### Intermediate 224: (R)—N-(2-(1-methylpyrrolidin-2-yl)-1H-pyrrolo[3,2-c]pyridin-6-yl)-3-(1-(3-(piperidin-4-yl) propyl)-1H-pyrazol-4-yl)benzamide dihydrochloride

To a solution of tert-butyl (R)-6-(3-(1-(3-(1-(tert-butoxycarbonyl)piperidin-4-yl)propyl)-1H-pyrazol-4-yl)benzamido)-2-(1-methylpyrrolidin-2-yl)-1H-pyrrolo[3,2-c]pyridine-1-carboxylate (Intermediate 223, 0.28 g, 0.39 mmol) in 1,4-dioxane (8 mL) was added 4M hydrochloric acid in 1,4-dioxane (8 mL) was at 0° C. and the reaction mixture was stirred at room temperature for 16 h. The reaction mixture was concentrated in vacuo to afford (R)—N-(2-(1-methylpyrrolidin-2-yl)-1H-pyrrolo[3,2-c]pyridin-6-yl)-3-(1-(3-(piperidin-4-yl)propyl)-1H-pyrazol-4-yl)benzamide dihydrochloride (Intermediate 224, 0.29 g) as an off-white solid, which was used for next step without purification. ¹H NMR (400 MHz, DMSO-d₆) δ ppm 1.18-1.30 (m, 5H), 1.76-1.87 (m, 4H), 2.21 (s, 3H), 2.76-2.85 (m, 6H), 3.20-3.27 (m, 5H), 4.13-4.16 (m, 2H), 7.27 (s, 1H), 7.59 (t, J=7.76 Hz, 1H), 7.89 (d, J=7.50 Hz, 1H), 7.94 (d, J=8.16 Hz, 1H), 8.09 (s, 1H), 8.35 (s, 1H), 8.41 (s, 1H), 8.52 (s, 1H), 9.11 (s, 1H), 11.27 (s, 1H), 12.09 (br s, 1H), 13.18 (br s, 1H).

### Step 8

### Example 47: 3-(1-(3-(1-(2-(2,6-dioxopiperidin-3-yl)-1,3-dioxoisoindolin-4-yl)piperidin-4-yl)propyl)-1H-pyrazol-4-yl)-N-(2-((R)-1-methylpyrrolidin-2-yl)-1H-pyrrolo[3,2-c]pyridin-6-yl)benzamide

To a solution of (R)—N-(2-(1-methylpyrrolidin-2-yl)-1H-pyrrolo[3,2-c]pyridin-6-yl)-3-(1-(3-(piperidin-4-yl) propyl)-1H-pyrazol-4-yl)benzamide dihydrochloride (Intermediate 224, 0.22 g, 0.42 mmol) in DMSO (4 mL) was added 2-(2,6-dioxopiperidin-3-yl)-4-fluoroisoindoline-1,3-dione (CAS No: 835616-60-9, 0.118 g, 0.42 mmol) and diisopropylethylamine (0.225 mL 1.29 mmol) and the reaction mixture was heated at 110° C. for 6 h. The reaction mixture was poured into ice cold H₂O (40 mL) to get yellow precipitate, which was filtered and dried in vacuo to give crude material. The crude was purified by preparative HPLC (Method A) afford 3-(1-(3-(1-(2-(2,6-dioxopiperidin-3-yl)-1,3-dioxoisoindolin-4-yl)piperidin-4-yl)propyl)-1H-pyrazol-4-yl)-N-(2-((R)-1-methylpyrrolidin-2-yl)-1H-pyrrolo[3,2-c]pyridin-6-yl)benzamide (Example 47, 0.046 g, 14%) as a yellow solid. ¹H NMR (400 MHz, DMSO-d₆) δ ppm 1.23-1.37 (m, 4H), 1.77-1.91 (m, 7H), 1.99-2.03 (m, 1H), 2.13-2.15 (m, 1H), 2.17 (s, 3H), 2.23-2.30 (m, 1H), 2.44-2.48 (m, 1H), 2.53-2.60 (m, 2H), 2.82-2.87 (m, 3H), 3.12-3.16 (m, 1H), 3.30-3.34 (m, 1H), 3.66-3.70 (m, 2H), 4.16-4.18 (m, 2H), 5.05-5.10 (m, 1H), 6.40 (s, 1H), 7.02 (s, 1H), 7.29-7.33 (m, 2H), 7.48 (t, J=7.75 Hz, 1H), 7.64-7.68 (m, 1H), 7.77 (d, J=7.88 Hz, 1H), 7.84 (d, J=8.00 Hz, 1H), 8.02 (s, 1H), 8.23 (s, 1H), 8.31 (s, 1H), 8.34 (s, 1H), 8.52 (s, 1H), 10.51 (s, 1H), 11.38 (s, 1H). Mass spec: m/z: 668.3 [M+H]⁺.

### Example 48 was Made Following Scheme 46

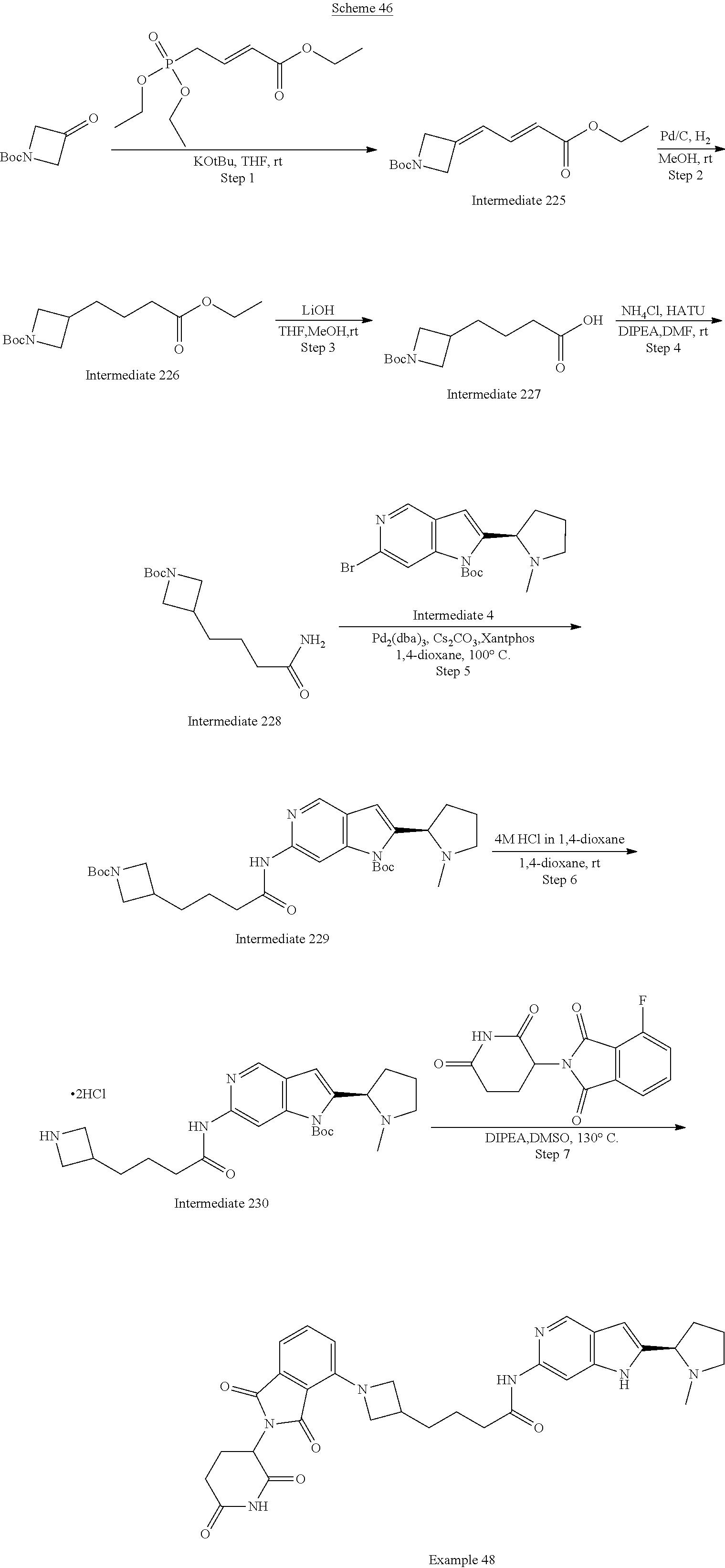

### Step 1

### Intermediate 225: tert-butyl (E)-3-(4-ethoxy-4-oxobut-2-en-1-ylidene) azetidine-1-carboxylate

To a solution of tert-butyl-3-oxoazetidine-1-carboxylate (CAS No: 398489-26-4, 5.00 g, 28.62 mmol) in tetrahydrofuran (50 mL) was added potassium tert-butoxide (2.00 g, 20 mmol) at 0° C. followed by triethyl 4-phosphonocrotonate (CAS No: 10236-14-3, 1.2 g, 4.30 mmol) and the reaction mixture was stirred at room temperature for 1 h. The reaction mixture was then filtered through a celite bed and the filtrate was concentrated in vacuo. The crude material was purified by combi-flash column chromatography, eluting with 35% EtOAc in heptane, to afford tert-butyl (E)-3-(4-ethoxy-4-oxobut-2-en-1-ylidene)azetidine-1-carboxylate (Intermediate 225, 1.1 g, 14%) as an off-white solid. ¹H NMR (400 MHz, DMSO-d₆) δ ppm 1.17 (t, J=6.8 Hz, 3H), 1.39 (s, 9H), 4.10-1.15 (m, 2H), 4.52 (s, 2H), 4.65 (s, 2H), 5.94 (d, J=15.34 Hz, 1H), 6.22 (d, J=11.20 Hz, 1H), 7.01-7.08 (m, 1H).

### Step 2

### Intermediate 226: tert-butyl 3-(4-ethoxy-4-oxobutyl) azetidine-1-carboxylate

To a solution of tert-butyl 3-[(E)-4-ethoxy-4-oxo-but-2-enylidene]azetidine-1-carboxylate (Intermediate 225, 1.1 g, 4.10 mmol) in methanol (45 mL) was added 10% palladium on carbon (0.60 g, 5.63 mmol). The reaction mixture was stirred at room temperature for 5 h under H₂ atmosphere at 50 psi. The reaction mixture was filtered through a celite bed and filtrate was concentrated in vacuo to afford tert-butyl 3-(4-ethoxy-4-oxo-butyl)azetidine-1-carboxylate (Intermediate 226, 1.00 g, 90%) as a colorless liquid, which used in the next step without further purification. ¹H NMR (400 MHz, DMSO-d₆) δ ppm 1.17 (t, J=7.09 Hz, 3H), 1.36 (s, 9H), 1.42-1.52 (m, 4H), 2.27 (t, J=7.1 Hz, 2H), 2.42-2.45 (m, 1H), 3.39-3.41 (m, 2H), 3.86-3.88 (m, 2H), 4.02-4.07 (m, 2H). Mass spec: m/z 272.1 [M+H]⁺.

### Step 3

### Intermediate 227: 4-(1-(tert-butoxycarbonyl)azetidin-3-yl)butanoic acid

To a solution of tert-butyl 3-(4-ethoxy-4-oxo-butyl)azetidine-1-carboxylate (Intermediate 226, 1.4 g, 5.2 mmol) in tetrahydrofuran (10.0 mL) and methanol (5 mL) was added lithium hydroxide (0.25 g, 10 mmol) in water (2 mL) at room temperature and stirred for 2 h. The reaction mixture was then acidified with aqueous citric acid and the pH was adjusted to ~5, upon which a solid precipitated. The solid was filtered and washed with water and dried under vacuum to afford 4-(1-(tert-butoxycarbonyl)azetidin-3-yl)butanoic acid (Intermediate 227, 1.20 g, 96%) as an off white solid. ¹H NMR (400 MHz, DMSO-d₆) δ ppm 1.36 (s, 9H), 1.41-1.43 (m, 2H), 1.48-1.52 (m, 2H), 2.20 (t, J=6.85 Hz, 2H), 2.43-2.45 (m, 1H), 3.39-3.41 (m, 2H), 3.86-3.89 (m, 2H), 12.10 (br s, 1H). Mass spec: m/z 244.1 [M+H]⁺.

### Step 4

### Intermediate 228: tert-butyl 3-(4-amino-4-oxobutyl)azetidine-1-carboxylate

To a solution of 4-(1-(tert-butoxycarbonyl)azetidin-3-yl)butanoic acid (Intermediate 227, 1.00 g, 4.0 mmol) in DMF (7 mL) was added HATU (2.3 g, 6.0 mmol) at room temperature and stirred for 15 min. Ammonium chloride (0.85 g, 16.0 mmol) was then added followed by diisopropylethylamine (2.1 mL, 12.0 mmol) and the reaction mixture was stirred at room temperature for 16 h. The reaction mixture was then diluted with water (50 mL) and extracted with EtOAc (3×50 mL), dried over Na₂SO₄, and concentrated in vacuo. The crude material was purified by combi-flash column chromatography eluting with 80% EtOAc in heptane to afford tert-butyl 3-(4-amino-4-oxobutyl)azetidine-1-carboxylate (Intermediate 228, 0.25 g, 26%) as a colourless liquid. ¹H NMR (400 MHz, DMSO-d₆) δ ppm 1.23-1.29 (m, 2H), 1.35 (s, 9H), 1.42-1.45 (m, 2H), 2.00 (t, J=7.34 Hz, 2H), 2.66-2.73 (m, 1H), 3.04-3.14 (m, 2H), 3.34-3.39 (m, 2H), 7.19 (br s, 2H). Mass spec: m/z 243.2 [M+H]⁺.

### Step 5

### Intermediate 229: tert-butyl (R)-6-(4-(1-(tert-butoxycarbonyl)azetidin-3-yl)butanamido)-2-(1-methylpyrrolidin-2-yl)-1H-pyrrolo[3,2-c]pyridine-1-carboxylate

To a solution of tert-butyl 3-(4-amino-4-oxobutyl)azetidine-1-carboxylate (Intermediate 228, 0.20 g, 0.84 mmol) 1,4-dioxane (5 mL) was added tert-butyl (R)-6-bromo-2-(1-methylpyrrolidin-2-yl)-1H-pyrrolo[3,2-c]pyridine-1-carboxylate (Intermediate 4, 0.15 g, 0.27 mmol) followed by cesium carbonate (1.03 g, 3.15 mmol) and the reaction mixture was purged with argon for 20 min. Pd₂(dba)₃ (0.148 g, 0.1578 mmol) followed by Xantphos (0.31 g, 0.188 mmol) was then added. The reaction mixture was further purged with argon gas for 10 minutes then heated at 100° C. for 2 h. The reaction mixture was then filtered through a celite bed and concentrated in vacuo. The crude material was purified by combi-flash column chromatography, by eluting with 85% EtOAc in heptane, to afford tert-butyl (R)-6-(4-(1-(tert-butoxycarbonyl)azetidin-3-yl)butanamido)-2-(1-methylpyrrolidin-2-yl)-1H-pyrrolo[3,2-c]pyridine-1-carboxylate (Intermediate 229, 0.15 g, 26%) as a yellow solid. ¹H NMR (400 MHz, DMSO-d₆) δ ppm 1.36 (s, 9H), 1.60 (s, 9H), 1.64-1.71 (m, 6H), 2.33-2.37 (m, 8H) 2.73 (s, 3H), 3.97-3.90 (m, 4H), 6.70 (br s, 1H), 8.79 (s, 1H), 9.14 (s, 1H), 10.35 (br s, 1H). Mass spec: m/z 542.3 [M+H]⁺.

### Step 6

### Intermediate 230: (R)-4-(azetidin-3-yl)-N-(2-(1-methylpyrrolidin-2-yl)-1H-pyrrolo[3,2-c]pyridin-6-yl) butanamide dihydrochloride

To a solution of tert-butyl (R)-6-(4-(1-(tert-butoxycarbonyl)azetidin-3-yl)butanamido)-2-(1-methylpyrrolidin-2-yl)-1H-pyrrolo[3,2-c]pyridine-1-carboxylate (Intermediate 229, 0.18 g, 0.40 mmol) in 1,4-dioxane (5.0 mL) was added 4M hydrochloric acid in 1,4-dioxane (5.0 mL) at 0° C. and then stirred at room temperature for 16 h. The reaction mixture was then concentrated in vacuo to afford (R)-4-(azetidin-3-yl)-N-(2-(1-methylpyrrolidin-2-yl)-1H-pyrrolo[3,2-c]pyridin-6-yl)butanamide dihydrochloride (Intermediate 230, 0.13 g) as a yellow solid, which was used in next step without further purification.

### Step 7

### Example 48: 4-(1-(2-(2,6-dioxopiperidin-3-yl)-1,3-dioxoisoindolin-4-yl)azetidin-3-yl)-N-(2-((R)-1-methylpyrrolidin-2-yl)-1H-pyrrolo[3,2-c]pyridin-6-yl)butanamide

To a solution of (R)-4-(azetidin-3-yl)-N-(2-(1-methylpyrrolidin-2-yl)-1H-pyrrolo[3,2-c]pyridin-6-yl) butanamide dihydrochloride (Intermediate 230, 0.15 g, 0.44 mmol) in dimethyl sulfoxide (1.0 mL) was added 2-(2,6-dioxopiperidin-3-yl)-4-fluoroisoindoline-1,3-dione (CAS No: 835616-60-9, 0.12 g, 0.43 mmol) followed by N,N-diisopropylethylamine (0.38 mL, 2.19 mmol) and the reaction mixture was heated at 130° C. for 2 h. The reaction mixture was then poured into ice cold water, upon which a solid precipitated, which was filtered and dried to obtain a crude product. The crude product was further purified by preparative HPLC (Method A) to afford 4-(1-(2-(2,6-dioxopiperidin-3-yl)-1,3-dioxoisoindolin-4-yl)azetidin-3-yl)-N-(2-((R)-1-methylpyrrolidin-2-yl)-1H-pyrrolo[3,2-c]pyridin-6-yl)butanamide (Example 48, 0.02 g, 8%) as a yellow solid. ¹H NMR (400 MHz, DMSO-d₆) δ ppm 1.59-1.63 (m, 4H), 1.76-1.90 (m, 3H), 1.97-2.00 (m, 1H), 2.09-2.18 (m, 1H), 2.25 (s, 3H), 2.39 (t, J=6.79 Hz, 2H), 2.55-2.59 (m, 1H), 2.67-2.70 (m, 1H), 2.82-2.91 (m, 1H), 3.10-3.14 (m, 1H), 3.24-3.28 (m, 2H), 3.73-3.88 (m, 2H), 4.31-4.33 (m, 2H), 5.01-5.05 (m, 1H), 6.33 (s, 1H), 6.76-6.77 (m, 1H), 7.09 (d, J=6.85 Hz, 1H), 7.53-7.57 (m, 1H), 8.08 (s, 1H), 8.40 (s, 1H), 10.14 (s, 1H), 11.25 (s, 1H). Mass spec: m/z 598.1 [M+H]⁺.

### Example 49 was Made Following Scheme 47

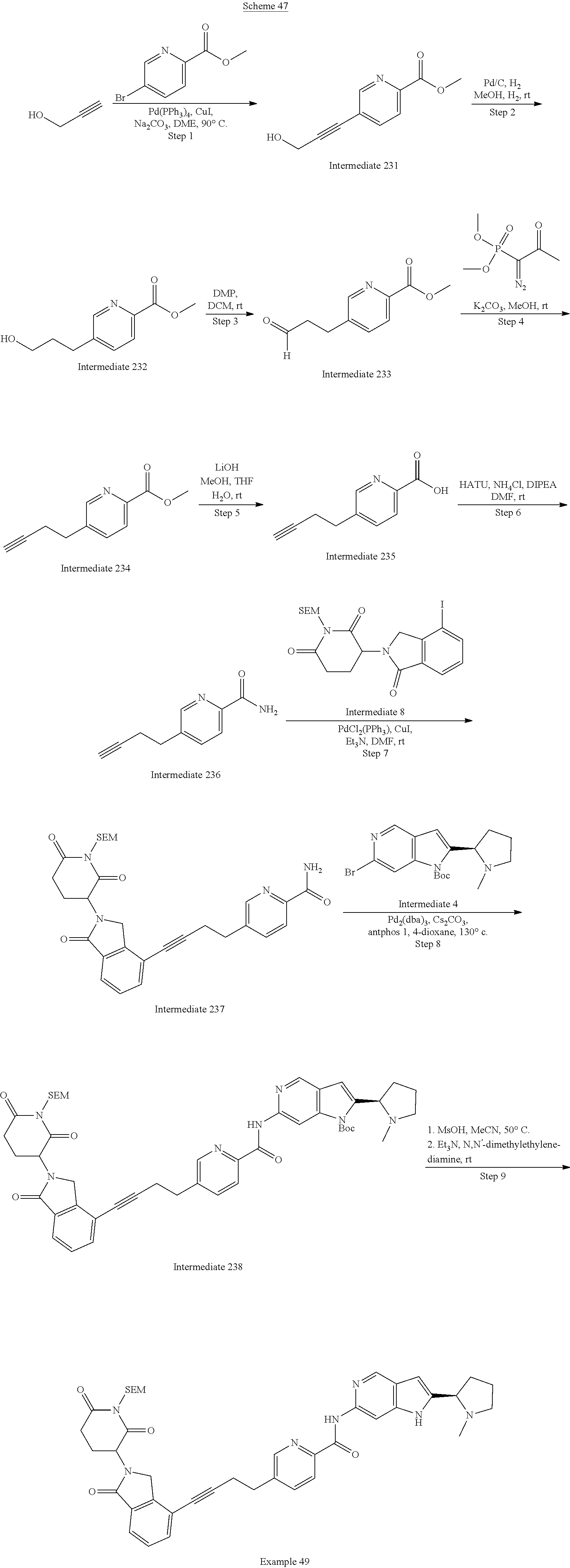

### Step 1

### Intermediate 231: methyl 5-(3-hydroxyprop-1-yn-1-yl)picolinate

To a solution of methyl 5-bromopicolinate (CAS No: 29682-15-3, 10 g, 46.28 mmol) in 1,2-Dimethoxyethane (100 mL) was added propargyl alcohol (3.9 g, 69.40 mmol) and sodium carbonate (15.6 g, 185.12 mmol). The resulting reaction mixture was degassed for 10 min with argon gas and to this, Pd(PPh₃)₄ (5.34 g, 4.63 mmol) and copper iodide (0.88 g, 4.63 mmol) were added sequentially at room temperature and again degassed for 5 min with argon gas. The reaction mixture was heated at 90° C. for 16 h. Progress of the reaction was monitored by TLC. After completion, the reaction mixture was filtered through celite, filtrate was diluted with water (50 mL) and extracted with EtOAc (200 mL). The organic layer was dried over Na₂SO₄ and concentrated in vacuo. The crude material was purified by combi-flash column chromatography, eluting with 30% EtOAc in heptane, to afford methyl 5-(3-hydroxyprop-1-ynyl)pyridine-2-carboxylate (Intermediate 231, 5.1 g, 59%) as a brown solid. ¹H NMR (400 MHz, DMSO-d₆) δ ppm 3.88 (s, 3H), 4.36 (d, J=5.6 Hz, 2H), 5.47 (t, J=6.0 Hz, 1H), 8.06-8.03 (m, 2H), 8.74 (s, 1H). Mass spec: m/z: 191.9 [M+H]⁺.

### Step 2

### Intermediate 232: methyl 5-(3-hydroxypropyl)picolinate

To a solution of methyl 5-(3-hydroxyprop-1-yn-1-yl)picolinate (Intermediate 231, 5 g, 26.15 mmol) in methanol (80 mL) was added 10% by wt. Pd/C (900 mg, 8.45 mmol) under nitrogen atmosphere and the resulting solution was stirred under H₂ atmosphere (120 psi) at room temperature for 16 h. The progress of reaction was monitored by TLC. After completion, the mixture was filtered through celite pad and filtrate was concentrated to give the crude mixture which was purified by combi-flash column chromatography, by eluting with 85% EtOAc in heptane, to afford methyl 5-(3-hydroxypropyl)picolinate (Intermediate 232, 2.4 g, 47%) as a brown liquid. ¹H NMR (400 MHz, DMSO-d₆) δ ppm 1.71-1.77 (m, 2H), 2.72 (t, J=8.0 Hz, 2H), 3.39-3.43 (m, 2H), 3.87 (s, 3H), 4.54 (t, J=5.2 Hz, 1H), 7.82 (d, J=7.2 Hz, 1H), 7.98 (d, J=7.2 Hz, 1H), 8.57 (s, 1H).

### Step 3

### Intermediate 233: methyl 5-(3-oxopropyl)picolinate

To a solution of methyl 5-(3-hydroxypropyl)picolinate (Intermediate 232, 1.0 g, 5.12 mmol) in dichloromethane (12 mL) was added Dess-Martin periodinane (4.34 g, 10.24 mmol) at 0° C. and the reaction mixture was stirred at room temperature for 5 h. The progress of the reaction was monitored by TLC. After completion of the reaction, the reaction mixture was diluted with water (60 mL) and extracted with EtOAc (2×60 mL). The organic layer was dried over Na₂SO₄ and concentrated to obtain crude methyl 5-(3-oxopropyl)picolinate (Intermediate 233, 1.0 g) as a white solid which was used for the next step without purification. ¹H NMR (400 MHz, DMSO-d₆) δ ppm 2.86-2.96 (m, 2H), 3.19-3.20 (m, 2H), 3.86 (s, 3H), 7.79-7.84 (d, J=8.4 Hz, 1H), 7.93-8.02 (m, 1H), 8.60 (s, 1H), 9.71 (s, 1H).

### Step 4

### Intermediate 243: methyl 5-(but-3-yn-1-yl)picolinate

To a solution of crude methyl 5-(3-oxopropyl)picolinate (Intermediate 233, 1.2 g, 6.21 mmol) in methanol (15 mL) was added potassium carbonate (1.3 g, 9.32 mmol) at 0° C. After stirring for 5 min (dimethyl (1-diazo-2-oxopropyl)phosphonate (1.8 g, 9.32 mmol) was added at the same temperature and stirred for 16 h. The progress of reaction was monitored by TLC. After completion of the reaction, mixture was diluted with water (50 mL) and extracted with EtOAc (3×40 mL). The organic layer was washed with brine (40 mL), dried over Na₂SO₄ and concentrated to give the crude material which was purified by combi-flash column chromatography, by eluting with 25% EtOAc in heptane, to afford methyl 5-(but-3-yn-1-yl)picolinate (Intermediate 234, 450 mg, 38%) as a colorless liquid. ¹H NMR (400 MHz, DMSO-d₆) δ ppm 2.50-2.55 (m, 2H), 2.81 (br s, 1H), 2.87 (t, J=7.2 Hz, 2H), 3.87 (s, 3H), 7.88 (d, J=8.0 Hz, 1H), 8.50 (d, J=8.0 Hz, 1H), 8.61 (s, 1H). Mass spec: m/z 190.1 [M+H]⁺.

### Step 5

### Intermediate 235: 5-(but-3-yn-1-yl) picolinic acid

To a solution of methyl 5-but-3-ynylpyridine-2-carboxylate (Intermediate 234, 450 mg, 2.39 mmol) in methanol (2 mL), and tetrahydrofuran (7 mL) was added lithium hydroxide (116 mg 4.76 mmol) in water (3 mL) at 0° C. and the reaction mixture was stirred at room temperature for 5 h. The progress of reaction mixture monitored by TLC. After completion of the reaction, solvent was evaporated under reduced pressure, water (20 mL) was added, acidified to pH 3-5 with 1 N HCl and the aqueous solution was extracted with 10% MeOH in DCM (3×40 mL). The organic layer was dried over Na₂SO₄ and concentrated under vacuum to obtain crude 5-but-3-ynylpyridine-2-carboxylic acid (Intermediate 235, 410 mg, 98%) as a white solid. ¹H NMR (400 MHz, DMSO-d₆) δ ppm 1.90 (s, 1H), 2.50-2.52 (m, 2H), 2.80-2.88 (m, 2H), 7.87 (d, J=7.60 Hz, 1H), 7.95 (d, J=7.60 Hz, 1H), 8.59 (s, 1H). Mass spec: m/z 176.1 [M+H]⁺.

### Step 6

### Intermediate 236: 5-(but-3-yn-1-yl)picolinamide

To a solution of 5-but-3-ynylpyridine-2-carboxylic acid (Intermediate 235, 430 mg, 2.45 mmol) in DMF (7 mL) were added HATU (1.39 g, 3.68 mmol), N,N-Diisopropylethylamine (1.30 mL, 7.36 mmol) and ammonium chloride (0.52 g, 9.81 mmol) at room temperature and stirred reaction mixture for 16 h. Progress of reaction was monitored by TLC. After completion of the reaction, mixture was diluted with water (60 mL) and extracted with 10% MeOH in DCM (3×40 mL). The organic layer was washed with ice cold water (60 ml) and brine (30 mL) and dried over Na₂SO₄ and concentrated in vacuo. The resulting crude residue was purified by combi-flash column chromatography, by eluting with 5% MeOH in DCM, to afford 5-(but-3-yn-1-yl)picolinamide (Intermediate 236, 400 mg, 94%) as a white solid. ¹H NMR (400 MHz, DMSO-d₆) δ ppm 2.50-2.54 (m, 2H), 2.81 (s, 1H), 2.85 (t, J=6.8 Hz, 2H), 7.56 (s, 1H), 7.86 (d, J=7.6 Hz, 1H), 7.92 (d, J=8.0 Hz, 1H), 8.06 (s, 1H), 8.52 (s, 1H). Mass spec: m/z 174.9 [M+H]⁺.

### Step 7

### Intermediate 237: 5-(4-(2-(2,6-dioxo-1-((2-(trimethylsilyl)ethoxy)methyl)piperidin-3-yl)-1-oxoisoindolin-4-yl)but-3-yn-1-yl)picolinamide

To a solution of 5-(but-3-yn-1-yl) picolinamide (Intermediate 236, 50 mg, 0.28 mmol) and 3-(4-iodo-1-oxo-isoindolin-2-yl)-1-(2-trimethylsilylethoxymethyl) piperidine-2,6-dione (Intermediate 8, 14 mg, 0.28 mmol) in DMF (2 mL) was added triethylamine (1.4 mL, 10.05 mmol) under argon. After purging for 10 min, copper iodide (5 mg 0.028 mmol) and Pd (PPh₃)₄Cl₂ (19 mg, 0.028 mmol) was added and stirred the reaction mixture at room temperature for 1 h. After completion of reaction, the reaction mixture was diluted with water (30 mL) and extracted with EtOAc (3×30 mL). The organic layer was washed with brine (2×30 mL), dried over Na₂SO₄ and concentrated to give the crude residue which was purified by combi-flash column chromatography, by eluting with EtOAc, to afford 5-(4-(2-(2,6-dioxo-1-((2-(trimethylsilyl)ethoxy)methyl)piperidin-3-yl)-1-oxoisoindolin-4-yl)but-3-yn-1-yl)picolinamide (Intermediate 237, 60 mg, 38%) as a sticky liquid. ¹H NMR (400 MHz, DMSO-d₆) δ ppm−0.04-0.05 (s, 9H), 0.81-0.85 (m, 2H), 2.72 (s, 2H), 2.80-2.83 (m, 2H), 2.84-2.86 (m, 2H), 2.89-2.97 (m, 2H), 2.98-3.07 (m, 1H), 3.51-3.55 (m, 2H), 4.10-4.25 (m, 2H), 5.02-5.10 (m, 2H), 7.49-7.58 (m, 4H), 7.70 (d, J=7.60 Hz, 1H), 7.92-8.05 (m, 3H). Mass spec: m/z 547.2 [M+H]⁺.

### Step 8

### Intermediate 238: tert-butyl 6-(5-(4-(2-(2,6-dioxo-1-((2-(trimethylsilyl)ethoxy)methyl)piperidin-3-yl)-1-oxoisoindolin-4-yl)but-3-yn-1-yl)picolinamido)-2-((R)-1-methylpyrrolidin-2-yl)-1H-pyrrolo[3,2-c]pyridine-1-carboxylate

To a solution of tert-butyl (R)-6-bromo-2-(1-methylpyrrolidin-2-yl)-1H-pyrrolo[3,2-c]pyridine-1-carboxylate (Intermediate 4, 250 mg, 0.65 mmol) in 1,4-dioxane (10 mL) was added 5-(4-(2-(2,6-dioxo-1-((2-(trimethylsilyl)ethoxy)methyl)piperidin-3-yl)-1-oxoisoindolin-4-yl)but-3-yn-1-yl)picolinamide (Intermediate 237, 0.287 g, 0.52 mmol) and cesium carbonate (0.646 g, 1.97 mmol) and the resulting mixture was degassed with argon gas for 10-20 min, then Pd₂(dba)₃ (93 mg, 0.098 mmol) followed by 4,5-Bis(diphenylphosphino)-9,9-dimethylxanthene (117 mg, 0.19 mmol) were added. The resulting reaction mixture was heated at 130° C. for 2 h. After completion of the reaction, the mixture was concentrated under reduced pressure to obtain crude material which was purified by combi-flash column chromatography, by eluting with 80% EtOAc in heptane, to obtain tert-butyl 6-(5-(4-(2-(2,6-dioxo-1-((2-(trimethylsilyl)ethoxy)methyl)piperidin-3-yl)-1-oxoisoindolin-4-yl)but-3-yn-1-yl)picolinamido)-2-((R)-1-methylpyrrolidin-2-yl)-1H-pyrrolo[3,2-c]pyridine-1-carboxylate (Intermediate 238, 200 mg, 41%) as a green solid. ¹H NMR (400 MHz, DMSO-d₆) δ ppm−0.070 (s, 9H), 0.71-0.74 (m, 2H), 1.62-1.65 (m, 2H), 1.62-1.66 (m, 2H), 1.72 (s, 9H), 2.27-2.39 (m, 6H), 2.40 (s, 3H), 2.89-2.95 (m, 2H), 3.06-3.13 (m, 4H), 3.34-3.36 (m, 1H), 3.62 (s, 2H), 3.91-3.94 (m, 1H), 4.31-4.39 (m, 2H), 6.76 (s, 1H), 7.46-7.51 (m, 1H), 7.62-7.62 (m, 1H), 7.65-7.67 (m, 1H), 8.09-8.11 (m, 1H), 8.20-8.23 (m, 1H), 8.58 (s, 1H), 8.75 (d, J=5.60 Hz, 1H), 8.98 (s, 1H), 10.44 (s, 1H).

### Step 9

### Example 49: 5-(4-(2-(2,6-dioxopiperidin-3-yl)-1-oxoisoindolin-4-yl)but-3-yn-1-yl)-N-(2-((R)-1-methylpyrrolidin-2-yl)-1H-pyrrolo[3,2-c]pyridin-6-yl)picolinamide

To a solution of tert-butyl 6-(5-(4-(2-(2,6-dioxo-1-((2-(trimethylsilyl)ethoxy)methyl)piperidin-3-yl)-1-oxoisoindolin-4-yl)but-3-yn-1-yl)picolinamido)-2-((R)-1-methylpyrrolidin-2-yl)-1H-pyrrolo[3,2-c]pyridine-1-carboxylate (Intermediate 238, 200 mg, 0.23 mmol) in MeCN (10 mL) was added methane sulfonic acid (347 mg, 3.54 mmol) at room temperature and heated at 50° C. for 2 h. After 2 h the reaction was cooled to room temperature and then N,N′-dimethylethylenediamine (138 mg, 1.41 mmol) followed by triethylamine (481 mg, 4.72 mmol) were added at room temperature and stirred for 3 h. The reaction mixture was concentrated under reduced pressure to obtain crude compound which was purified by preparative HPLC (Method A) to afford tert-butyl 6-(5-(4-(2-(2,6-dioxo-1-((2-(trimethylsilyl)ethoxy)methyl)piperidin-3-yl)-1-oxoisoindolin-4-yl)but-3-yn-1-yl)picolinamido)-2-((R)-1-methylpyrrolidin-2-yl)-1H-pyrrolo[3,2-c]pyridine-1-carboxylate (Example 49, 3 mg, 2%) as an off-white solid. ¹H NMR (400 MHz, DMSO-d₆) δ ppm 1.81-189 (m, 5H), 2.17 (brs, 3H), 2.88-2.91 (m, 6H), 3.02-3.13 (m, 3H), 4.54-4.58 (m, 4H), 6.57 (s, 1H), 7.45-7.48 (m, 2H), 7.52 (d, J=6.8 Hz, 1H), 7.63 (d, J=6.8 Hz, 1H), 8.04-8.05 (m, 2H), 8.16 (d, J=8.0 Hz, 1H), 8.24-8.26 (m, 1H), 8.51-8.55 (m, 2H), 8.73 (s, 1H). Mass spec: m/z 616.3 [M+H]⁺.

### Example 50 was Synthesised Following Scheme 48

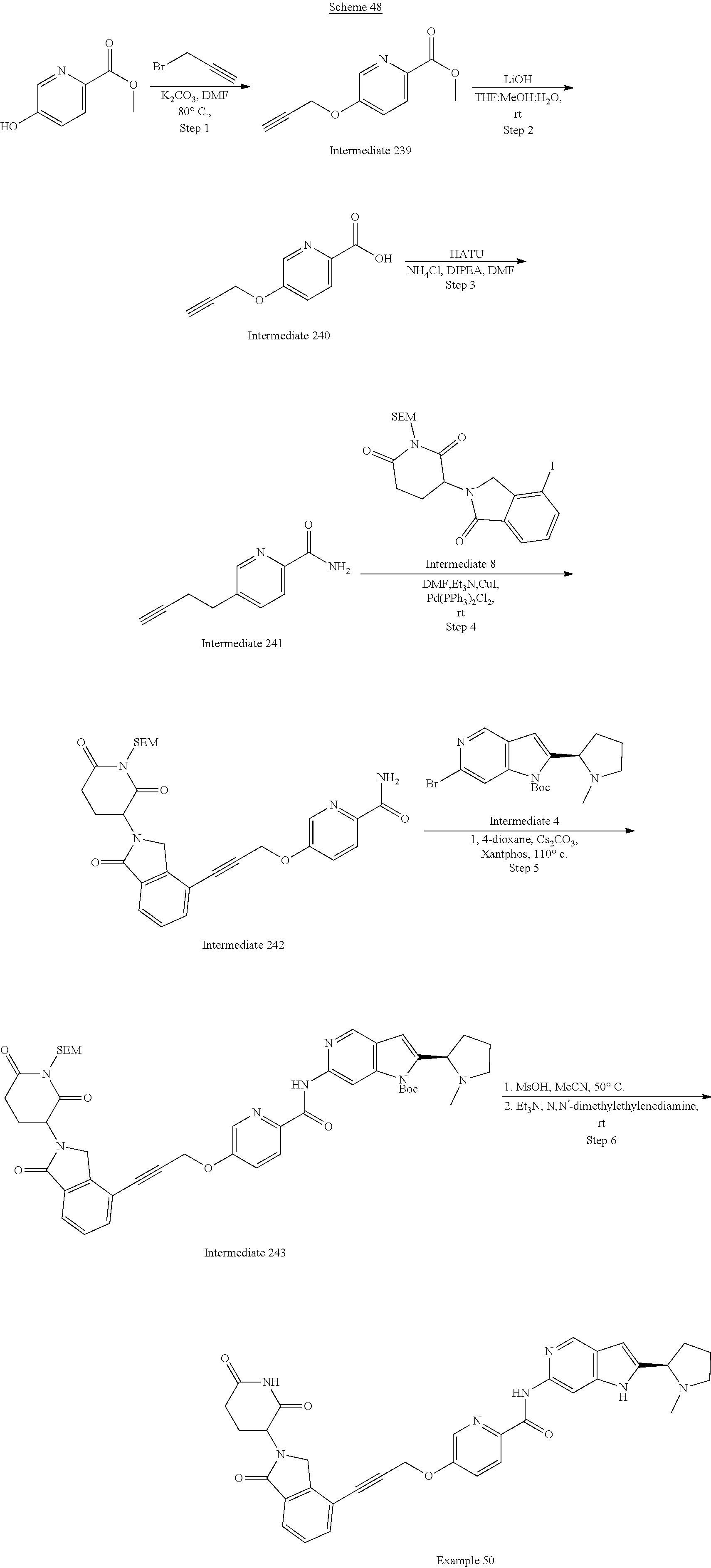

### Step 1

### Intermediate 239: methyl 5-(prop-2-yn-1-yloxy)picolinate

To a solution of methyl 5-hydroxypyridine-2-carboxylate (CAS No: 30766-12-2, 4.0 g, 26.12 mmol) in DMF (30 mL) was added potassium carbonate (5.42 g, 39.18 mmol). The reaction mixture was stirred at room temperature for 15 min then 3-bromo-1-propyne (CAS No: 106-96-7, 4.66 g, 31.34 mmol) was added at room temperature and the reaction mixture was heated at 80° C. for 16 h. Progress of the reaction was monitored by TLC. After completion, reaction mixture was diluted with water (50 mL) and extracted with EtOAc (3×60 mL). The organic layer was washed with NaHCO₃ (2×50 mL) and brine (2×50 mL). The organic layers were separated and dried over Na₂SO₄ and concentrated to give crude product. The crude product was purified by column chromatography by eluting with 70% EtOAc in heptane to afford methyl 5-(prop-2-yn-1-yloxy)picolinate (Intermediate 239, 2.5 g, 50%) as an off-white solid. ¹H NMR (400 MHz, DMSO-d₆) δ ppm 3.69 (s, 1H) 3.85 (s, 3H) 5.01 (d, J=2.49 Hz, 2H) 7.53-7.63 (m, 1H) 8.07 (d, J=8.71 Hz, 1H) 8.42 (d, J=2.90 Hz, 1H). Mass spec: m/z 191.9 [M+H]⁺.

### Step 2

### Intermediate 240: 5-(prop-2-yn-1-yloxy)picolinic acid

To a solution of methyl 5-(prop-2-yn-1-yloxy)picolinate (Intermediate 239, 2 g, 10.46 mmol) in THF (7 mL) and methanol (7 mL) was added lithium hydroxide (1.02 g, 41.84 mmol) in water (7 mL) at room temperature and the reaction mixture was stirred at RT for 16 h. Progress of the reaction was monitored by TLC. After completion of reaction solvent was evaporated under reduced pressure, water (15 mL) was added, acidified to pH 2-3 with 1N HCl at 0° C. to obtain white precipitate, which was filtered and dried to afford 5-(prop-2-yn-1-yloxy)picolinic acid (Intermediate 240, 1.8 g, 91%) as an off-white solid. ¹H NMR (400 MHz, DMSO-d₆) δ ppm 3.68 (s, 1H) 4.99 (s, 2H) 7.54-7.57 (m, 1H) 8.04 (d, J=8.71 Hz, 1H) 8.38 (d, J=2.49 Hz, 1H) 12.20 (br s, 1H). Mass spec: m/z 178.0 [M+H]⁺.

### Step 3

### Intermediate 241: 5-(prop-2-yn-1-yloxy)picolinamide

To a solution of 5-(prop-2-yn-1-yloxy)picolinic acid (Intermediate 240, 1.8 g, 10 mmol) in DMF (20 mL) was added HATU (8.0 g, 20 mmol) and N,N-Diisopropylethylamine (4.6 g, 36 mmol). Reaction mixture was stirred at room temperature for 15 min then added ammonium chloride (3.8 g, 71 mmol). Reaction mixture was stirred at room temperature for 16 h. Progress of the reaction was monitored by TLC. The reaction mixture was diluted with water (20 mL), extracted with EtOAc (2×50 mL). The organic layer was washed with NaHCO₃ (2×30 mL) and brine (30 mL), dried over Na₂SO₄ and concentrated to give crude. The crude material was purified by combi-flash eluted with 2% MeOH in DCM to afford 5-(prop-2-yn-1-yloxy)picolinamide (Intermediate 241, 1.3 g, 73%) as a white solid. ¹H NMR (400 MHz, DMSO-d₆) δ ppm 3.68 (s, 1H), 4.98 (s, 2H), 7.49 (br s, 1H), 7.56-7.58 (m, 1H), 7.94 (br s, 1H), 8.00-8.02 (m, 1H), 8.33 (s, 1H). Mass spec: m/z 177.1 [M+H]⁺.

### Step 4

### Intermediate 242: 5-((3-(2-(2,6-dioxo-1-((2-(trimethylsilyl)ethoxy)methyl)piperidin-3-yl)-1-oxoisoindolin-4-yl)prop-2-yn-1-yl)oxy)picolinamide

To a solution of 5-(prop-2-yn-1-yloxy)picolinamide (Intermediate 241, 0.50 g, 2.83) and 3-(4-iodo-1-oxo-isoindolin-2-yl)-1-(2-trimethylsilylethoxymethyl)piperidine-2,6-dione (Intermediate 8, 1.42 g, 2.83 mmol) in DMF (20 mL) were added triethylamine (10.1 g, 99.33 mmol), dichlorobis(triphenylphosphine)palladium(II) (0.20 g, 0.28 mmol) and copper (I) iodide (0.055 g, 0.28 mmol) at rt. Reaction mixture was stirred at RT for 2 h. Progress of the reaction was monitored by TLC. The reaction mixture was diluted with water (50 mL) and extracted with EtOAc (3×60 mL). The organic layer was washed with brine (2×50 mL), dried over Na₂SO₄ and concentrated to give crude. The crude product was purified by combi-flash column by eluting with 100% EtOAc to afford 5-((3-(2-(2,6-dioxo-1-((2-(trimethylsilyl)ethoxy)methyl)piperidin-3-yl)-1-oxoisoindolin-4-yl)prop-2-yn-1-yl)oxy)picolinamide (Intermediate 242, 0.65 g, 42%) as a yellow solid. ¹H NMR (400 MHz, DMSO-d₆) δ ppm −0.09-0.05 (m, 9H), 0.75-0.92 (m, 2H), 1.98-2.09 (m, 1H), 2.27-2.37 (m, 1H), 2.81-2.82 (m, 1H), 3.00-3.14 (m, 1H), 3.52-3.55 (m, 2H), 4.10-4.22 (m, 1H), 4.26-4.40 (m, 1H), 5.13 (s, 2H), 5.21-5.34 (m, 3H), 7.50 (br s, 1H), 7.53-7.61 (m, 1H), 7.66-7.75 (m, 2H), 7.79 (d, J=7.67 Hz, 1H), 7.94 (br s, 1H), 7.99-8.09 (m, 1H), 8.40 (s, 1H). Mass spec: m/z 549.4 [M+H]⁺.

### Step 5

### Intermediate 243: tert-butyl 6-(5-((3-(2-(2,6-dioxo-1-((2-(trimethylsilyl)ethoxy)methyl)piperidin-3-yl)-1-oxoisoindolin-4-yl)prop-2-yn-1-yl)oxy)picolinamido)-2-((R)-1-methylpyrrolidin-2-yl)-1H-pyrrolo[3,2-c]pyridine-1-carboxylate

To a solution 5-((3-(2-(2,6-dioxo-1-((2-(trimethylsilyl)ethoxy)methyl)piperidin-3-yl)-1-oxoisoindolin-4-yl)prop-2-yn-1-yl)oxy)picolinamide (Intermediate 242, 0.60 g, 1.09 mmol) in 1,4-dioxane (7 mL) was added tert-butyl 6-bromo-2-(1-methylpyrrolidin-2-yl)pyrrolo[3,2-c]pyridine-1-carboxylate (Intermediate 4, 0.49 g, 1.31 mmol) and cesium carbonate (1.07 g, 3.28 mmol) at room temperature and the reaction mixture was degassed with argon gas for 10 min then added xantphos (0.19 g, 0.32 mmol) and Pd₂ (dba)₃ (0.15 g, 0.16 mmol). The reaction mixture was heated at 110° C. for 2 h. Progress of the reaction was monitored by TLC. The reaction mixture was diluted with water (50 mL) and extracted with EtOAc (3×60 mL). The combined organic layer was washed with brine (2×50 mL), dried over Na₂SO₄ and concentrated to get crude. The crude product was purified by combi-flash column eluted with 2% MeOH in DCM to afford tert-butyl 6-[[5-[3-[2-[2,6-dioxo-1-(2-trimethylsilylethoxymethyl)-3-piperidyl]-1-oxo-isoindolin-4-yl]prop-2-ynoxy]pyridine-2-carbonyl]amino]-2-(1 methylpyrrolidin-2 yl)pyrrolo[3,2-c]pyridine-1-carboxylate (Intermediate 243, 0.32 g, 35%) as a yellow solid. ¹H NMR (400 MHz, DMSO-d₆) δ ppm −0.07 (s, 9H), 0.71-0.86 (m, 2H), 1.62 (m, 3H), 1.72 (s, 9H), 2.06-2.13 (m, 1H), 2.30-2.33 (m, 4H), 2.35 (s, 3H), 2.74-2.78 (m, 1H), 3.01-3.12 (m, 2H), 3.41-3.53 (m, 3H), 3.89-3.92 (m, 1H), 4.08-4.36 (m, 2H), 4.93-5.03 (m, 2H), 5.21-5.23 (m, 1H), 5.37 (s, 2H), 6.76 (s, 1H), 7.52-7.63 (m, 1H), 7.73-7.83 (m, 3H), 8.24 (d, J=8.71 Hz, 1H), 8.56-8.58 (m, 2H), 8.93-8.99 (m, 1H), 10.30 (s, 1H). Mass spec: m/z 848.4 [M+H]⁺.

### Step 6

### Example 50: 5-[3-[2-(2,6-dioxo-3-piperidyl)-1-oxo-isoindolin-4-yl]prop-2-ynoxy]-N-[2-(1-methylpyrrolidin-2-yl)-1H-pyrrolo[3,2-c]pyridin-6-yl]pyridine-2-carboxamide

To a solution of tert-butyl 6-[[5-[3-[2-[2,6-dioxo-1-(2-trimethylsilylethoxymethyl)-3-piperidyl]-1-oxo-isoindolin-4-yl]prop-2-ynoxy]pyridine-2-carbonyl]amino]-2-(1-methylpyrrolidin-2-yl)pyrrolo[3,2-c]pyridine-1-carboxylate (Intermediate 243, 0.30 g, 0.353 mmol) in acetonitrile (8 mL) was added methane sulfonic acid (0.52 g, 5.30 mmol) at room temperature and the reaction mixture was heated at 50° C. for 2 h. To the reaction mixture triethylamine (0.72 g, 7.075 mmol) and N,N′-dimethylethylenediamine (0.20 g, 2.12 mmol) was added and the reaction mixture was stirred at RT for 2 h. Progress of the reaction was monitored by TLC. The reaction mixture was concentrated to get crude mixture. The crude compound was purified by preparative HPLC (Method A) to afford 5-[3-[2-(2,6-dioxo-3-piperidyl)-1-oxo-isoindolin-4-yl]prop-2-ynoxy]-N-[2-(1-methylpyrrolidin-2-yl)-1H-pyrrolo[3,2-c]pyridin-6-yl]pyridine-2-carboxamide (Example 50, 0.28 g, 13%) as an off-white solid. ¹H NMR (400 MHz, DMSO-d₆) δ ppm 1.78-1.95 (m, 3H), 1.97-2.06 (m, 1H), 2.18-2.20 (m, 3H), 2.30-2.44 (m, 1H), 2.54 (s, 3H), 2.61-2.69 (m, 1H), 2.86-2.96 (m, 1H), 3.08-3.20 (m, 1H), 4.25-4.48 (m, 2H), 5.10-5.18 (m, 1H), 5.36 (s, 2H), 6.42 (s, 1H), 7.48-7.62 (m, 1H), 7.69-7.84 (m, 3H), 8.17-8.30 (m, 2H), 8.49 (s, 1H), 8.57 (d, J=2.81 Hz, 1H), 10.17 (s, 1H), 11.14 (s, 1H), 11.46 (s, 1H). Mass spec: m/z 618.0 [M+H]⁺.

### Example 51 was Synthesised Following Scheme 49

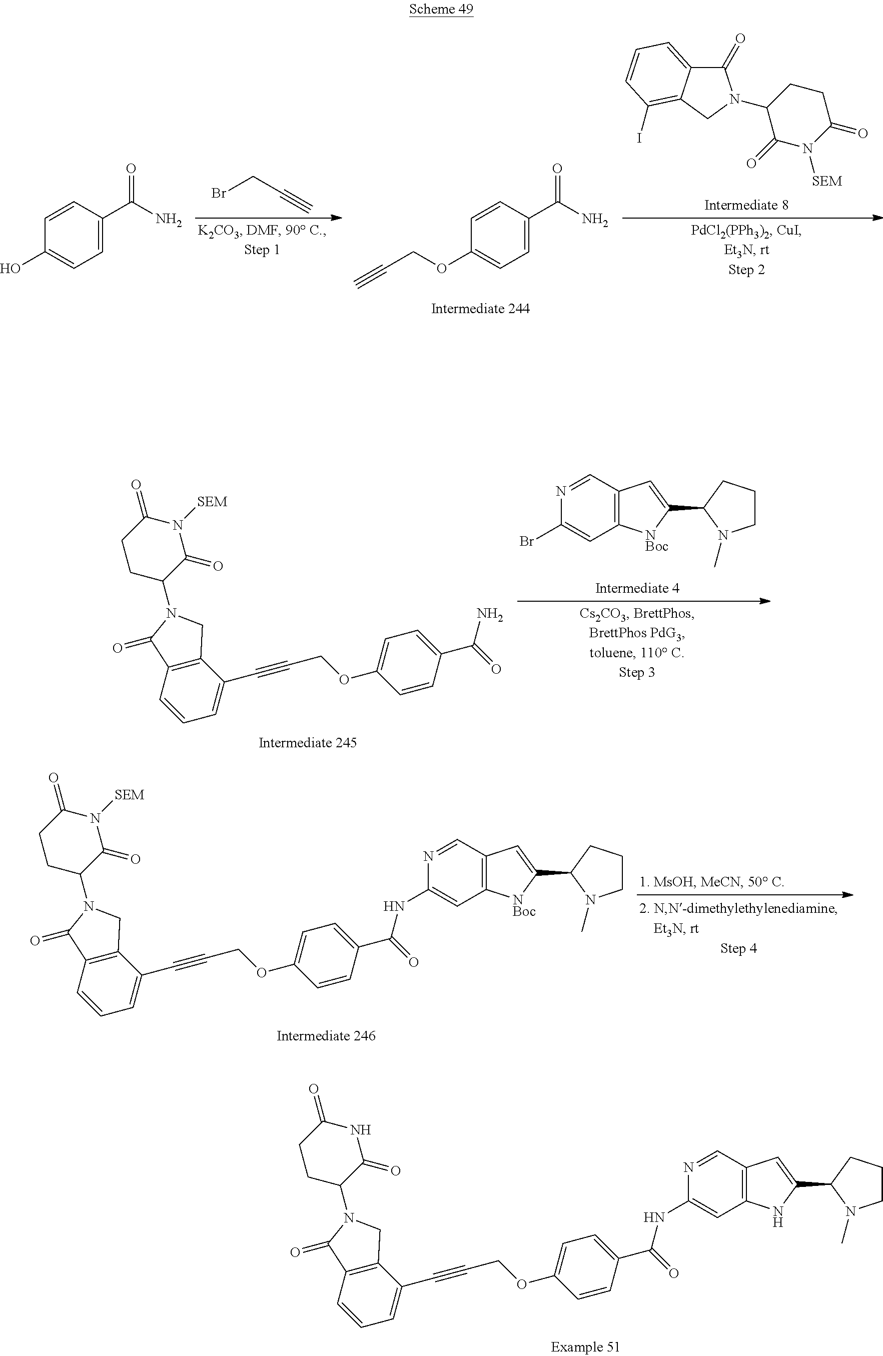

### Step 1

### Intermediate 244: 4-(prop-2-yn-1-yloxy)benzamide

To a solution of 4-hydroxybenzamide (CAS No: 619-57-8, 4 g, 29.16 mmol) in DMF (40 mL) was added potassium carbonate (6.05 g, 43.75 mmol) and propargyl bromide (CAS No: 106-96-7, 5.20 g, 35.00 mmol). The reaction mixture was heated at 90° C. for 12 h. Progress of the reaction was monitored by TLC. After completion, the reaction mixture was diluted with water (80 mL) and extracted with EtOAc (2×80 mL). The organic layer was washed with brine (3×60 mL). The organic layer was separated and dried over Na₂SO₄ and concentrated in vacuo. The obtained crude material was purified by combi-flash column chromatography, by eluting with 2% MeOH in DCM, to afford 4-(prop-2-yn-1-yloxy)benzamide (Intermediate 244, 2.6 g, 51%) as an off solid. ¹H NMR (400 MHz, DMSO-d₆) δ ppm 3.59 (s, 1H), 4.86 (s, 2H), 7.02 (d, J=8.80 Hz, 2H), 7.19 (br s, 1H), 7.85 (d, J=7.83 Hz, 2H), 8.66 (br s, 1H). Mass spec: m/z 176.1 [M+H]⁺.

### Step 2

### Intermediate 245: 4-((3-(2-(2,6-dioxo-1-((2-(trimethylsilyl) ethoxy)methyl) piperidin-3-yl)-1-oxoisoindolin-4-yl) prop-2-yn-1-yl)oxy)benzamide

To a solution of 3-(4-iodo-1-oxoisoindolin-2-yl)-1-((2-(trimethylsilyl)ethoxy)methyl)piperidine-2,6-dione (Intermediate 8, 1.5 g, 3.0 mmol) and 4-(prop-2-yn-1-yloxy)benzamide (Intermediate 244, 0.53 g, 3.0 mmol) in DMF (20 mL) was added triethylamine (15 mL, 100 mmol). The reaction mixture was purged with argon gas for 5 min then PdCl₂(PPh₃)₂ (0.21 g, 0.30 mmol) and Copper (I) iodide (0.05 g, 0.30 mmol) were added. The reaction mixture was stirred at room temperature for 2 h. Progress of the reaction was monitored by TLC. After completion, the reaction mixture was diluted with water (50 mL) and extracted with EtOAc (3×60 mL). The organic layer was washed with brine solution (3×60 mL). The organic layer was dried over Na₂SO₄ and concentrated in vacuo. The obtained crude material was purified by combi-flash column chromatography, by eluting with 90% EtOAc in heptane, to afford 4-((3-(2-(2,6-dioxo-1-((2-(trimethylsilyl)ethoxy)methyl)piperidin-3-yl)-1-oxoisoindolin-4-yl)prop-2-yn-1-yl)oxy)benzamide (Intermediate 245, 1.0 g, 61%) as a yellow solid. ¹H NMR (400 MHz, DMSO-d₆) δ ppm −0.03 (s, 9H), 0.83-0.85 (m, 2H), 1.97-2.04 (m, 1H), 2.27-2.32 (m, 1H), 2.78-2.82 (m, 1H), 3.03-3.09 (m, 1H), 3.53 (s, 2H), 4.11-4.16 (m, 1H), 4.29-4.34 (m, 1H), 5.04-5.09 (m, 2H), 5.17 (s, 2H), 5.22-5.26 (m, 1H), 7.11 (d, J=7.88 Hz, 2H), 7.20 (br s, 1H), 7.54-7.58 (m, 1H), 7.71 (d, J=7.46 Hz, 1H), 7.78-7.79 (m, 1H), 7.83 (br s, 1H), 7.87 (d, J=8.29 Hz, 2H).

### Step 3

### Intermediate 246: tert-butyl 6-(4-((3-(2-(2,6-dioxo-1-((2-(trimethylsilyl)ethoxy)methyl)piperidin-3-yl)-1-oxoisoindolin-4-yl)prop-2-yn-1-yl)oxy)benzamido)-2-((R)-1-methylpyrrolidin-2-yl)-1H-pyrrolo[3,2-c]pyridine-1-carboxylate

To a solution of 4-((3-(2-(2,6-dioxo-1-((2-(trimethylsilyl)ethoxy)methyl)piperidin-3-yl)-1-oxoisoindolin-4-yl)prop-2-yn-1-yl)oxy)benzamide (Intermediate 245, 0.50 g, 0.9129 mmol) in toluene (10 mL) was added tert-butyl (R)-6-bromo-2-(1-methylpyrrolidin-2-yl)-1H-pyrrolo[3,2-c]pyridine-1-carboxylate (Intermediate 4, 0.41 g, 1.095 mmol) followed by caesium carbonate (0.89 g, 2.739 mmol) at room temperature. The reaction mixture was purged with argon for 10 min then BrettPhos (0.075 g, 0.1369 mmol) and BrettPhos PdG3 (0.12 g, 0.1369 mmol) were added, and the reaction mixture was again purged with argon gas for 5 min and heated at 110° C. for 2 h. Progress of the reaction was monitored by TLC. After completion, the reaction mixture was cooled to room temperature, treated with water (40 mL) and extracted with EtOAc (2×50 mL). The organic layer was separated and dried over Na₂SO₄ and concentrated in vacuo. The crude material was purified by combi-flash column chromatography, by eluting with 2% MeOH in DCM, to afford tert-butyl 6-(4-((3-(2-(2,6-dioxo-1-((2-(trimethylsilyl)ethoxy)methyl)piperidin-3-yl)-1-oxoisoindolin-4-yl)prop-2-yn-1-yl)oxy)benzamido)-2-((R)-1-methylpyrrolidin-2-yl)-1H-pyrrolo[3,2-c]pyridine-1-carboxylate (Intermediate 246, 0.24 g, 31%) as an off-white solid. ¹H NMR (400 MHz, DMSO-d₆) δ ppm−0.07 (s, 9H), 0.76-0.80 (m, 2H), 1.17-1.19 (m, 1H), 1.70 (s, 9H), 1.98-2.05 (m, 2H), 2.13-2.15 (m, 1H), 2.28-2.31 (m, 3H), 2.35 (s, 3H), 2.74-2.78 (m, 1H), 2.99-3.09 (m, 1H), 3.36-3.48 (m, 2H), 3.90-3.94 (m, 1H), 3.99-4.07 (m, 1H), 4.09-4.18 (m, 1H), 4.29-4.37 (m, 1H), 4.95-5.02 (m, 2H), 5.19-5.26 (m, 3H), 6.75 (s, 1H), 7.17 (d, J=8.71 Hz, 2H), 7.54-7.60 (m, 1H), 7.73 (d, J=7.46 Hz, 1H), 7.78 (d, J=7.46 Hz, 1H), 8.09 (d, J=8.29 Hz, 2H), 8.58 (s, 1H), 8.93 (s, 1H), 10.54 (s, 1H). Mass spec: m/z 847.5 [M+H]⁺.

### Step 4

### Example 51: 4-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)benzamide

To a solution of tert-butyl 6-(4-((3-(2-(2,6-dioxo-1-((2-(trimethylsilyl)ethoxy)methyl)piperidin-3-yl)-1-oxoisoindolin-4-yl)prop-2-yn-1-yl)oxy)benzamido)-2-((R)-1-methylpyrrolidin-2-yl)-1H-pyrrolo[3,2-c]pyridine-1-carboxylate (Intermediate 246, 0.36 g, 0.42 mmol) in acetonitrile (10 mL) was added methanesulfonic acid (0.42 mL, 6.37 mmol) at room temperature and the reaction mixture was heated at 50° C. for 2 h. Reaction mixture was cooled to RT then triethylamine (1.19 mL, 8.50 mmol) added and N,N′-dimethylethylenediamine (0.30 mL, 2.55 mmol) and stirred at room temperature for 2 h. Progress of the reaction was monitored by TLC. After completion, the reaction mixture was concentrated in vacuo. The crude compound was purified by preparative HPLC (Method A) to afford 4-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)benzamide (Example 51, 0.03 g, 11%) as an off white solid. ¹H NMR (400 MHz, DMSO-d₆) δ ppm 1.75-1.94 (m, 3H), 1.97-2.04 (m, 1H), 2.11-2.15 (m, 1H), 2.17 (s, 3H), 2.20-2.30 (m, 1H), 2.38-2.44 (m, 1H), 2.59-2.63 (m, 2H), 2.86-2.96 (m, 1H), 3.10-3.18 (m, 1H), 4.26-4.33 (m, 1H), 4.37-4.45 (m, 1H), 5.13-5.18 (m, 1H), 5.22 (s, 2H), 6.40 (s, 1H), 7.18 (d, J=8.80 Hz, 2H), 7.54-7.59 (m, 1H), 7.72 (d, J=7.58, 1H), 7.78 (d, J=7.58, 1H), 8.07 (d, J=8.80 Hz, 2H), 8.19 (s, 1H), 8.49 (s, 1H), 10.28 (br s, 1H), 11.25 (br s, 1H), 11.37 (br s, 1H). Mass spec: m/z 617.2 [M+H]⁺.

### Example 52 was Synthesised Following Scheme 50

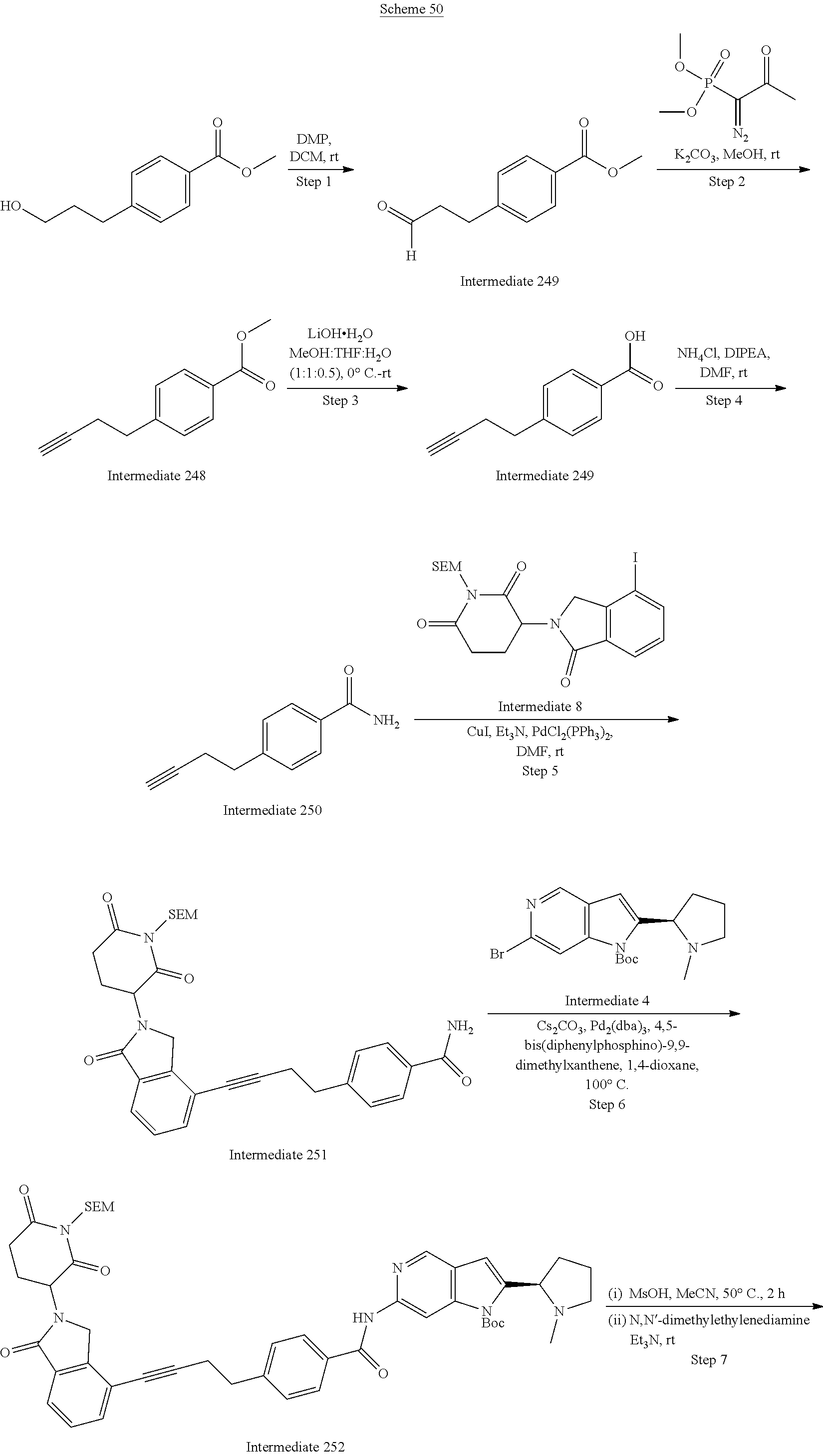

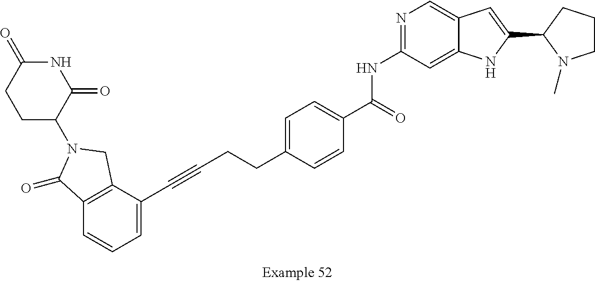 [SMILES: CN1CCC[C@@H]1c1cc2cnc(NC(=O)c3ccc(CCC#Cc4cccc5c4CN(C4CCC(=O)NC4=O)C5=O)cc3)cc2[nH]1]

### Step 1

### Intermediate 247: methyl 4-(3-oxopropyl)benzoate

To a solution of methyl 4-(3-hydroxypropyl)benzoate (CAS No: 15403-22-2, 5 g, 25.74 mmol) in dichloromethane (40 mL) was added Dess-Martin periodinane (14.63 g, 33.47 mmol) at 0° C. and the reaction mixture was stirred at room temperature for 6 h. The progress of reaction was monitored by TLC. After completion of the reaction, the mixture was diluted with saturated NaHCO₃ (100 mL) and saturated sodium thiosulphate (100 mL) and extracted with DCM (3×150 mL). The combined organic layer was separated, dried over anhydrous Na₂SO₄ and concentrated under reduced pressure to afford the crude methyl 4-(3-oxopropyl)benzoate (Intermediate 247, 7.0 g crude) as a yellow liquid which was used directly into next step without further purification. ¹H NMR (400 MHz, DMSO-d₆) δ ppm 2.83 (d, J=6.80 Hz, 2H), 2.92 (d, J=7.2 HZ, 2H), 3.83 (s, 3H), 7.37 (d, J=7.6 HZ, 2H), 7.87 (d, J=7.6 Hz, 2H), 9.71 (s, 1H). Mass spec: m/z 190.9 [M+H]⁺.

### Step 2

### Intermediate 248: methyl 4-(but-3-yn-1-yl)benzoate

To a solution of methyl 4-(3-oxopropyl)benzoate (Intermediate 247, 3 g, 15.61 mmol) in methanol (30 mL) was added K₂CO₃ (4.3 g, 31.22 mmol) at room temperature followed by 1-diazo-1-dimethoxyphosphoryl-propan-2-one (3.30 g, 17.17 mmol) was added. The resulting reaction mixture was stirred for 4 h. The progress of the reaction was monitored by TLC. After completion of the reaction, the mixture was diluted with water (500 mL) and extracted with diethyl ether (3×200 mL). The combined organic layer was separated, dried over anhydrous Na₂SO₄ and concentrated under reduced pressure to afford crude compound which was purified by combi-flash column chromatography, by eluting with 10% EtOAc in heptane, to afford methyl 4-(but-3-yn-1-yl)benzoate (Intermediate 248, 2.3 g, 78%) as a yellow liquid. ¹H NMR (500 MHz, DMSO-d₆) δ ppm 2.48 (s, 1H), 2.50-2.52 (m, 1H), 2.77-2.81 (m, 1H), 2.84 (t, J=7.2 Hz, 2H), 3.84 (s, 3H), 7.40 (d, J=8.0 Hz, 2H), 7.88 (d, J=8.0 Hz, 2H). Mass spec: m/z 189.1 [M+H]⁺.

### Step 3

### Intermediate 249: 4-(but-3-yn-1-yl)benzoic acid

To a solution of methyl 4-(but-3-yn-1-yl)benzoate (Intermediate 248, 2.3 g, 12 mmol) in tetrahydrofuran (10 mL) and methanol (10 mL) was added lithium hydroxide monohydrate (1.20 g, 49 mmol) in water (5 mL) at 0° C. and the resulting reaction mixture was stirred at room temperature for 6 h. The progress of the reaction was monitored by TLC. After completion of reaction, solvent was evaporated under reduced pressure, water (20 mL) was added. The aqueous solution was acidified with 0.5 N HCl to a pH~3-4, then extracted with EtOAc (2×250 mL). The combined organic layer was dried over Na₂SO₄ and concentrated. The crude solid obtained was triturated with n-pentane to afford 4-(but-3-yn-1-yl)benzoic acid (Intermediate 249, 1.9 g, 89%) as an off-white solid. ¹H NMR (500 MHz, DMSO-d₆) δ ppm 2.49-2.51 (m, 2H), 2.78 (s, 1H), 2.83 (t, J=6.8 Hz, 2H), 7.37 (d, J=7.6 Hz, 2H), 7.86 (d, J=6.8 Hz, 2H), 12.84 (br s, 1H). Mass spec: m/z 173.0 [M+H]⁺.

### Step 4

### Intermediate 250: 4-(but-3-yn-1-yl)benzamide

To a solution of 4-(but-3-yn-1-yl)benzoic acid (Intermediate 249, 1.9 g, 11 mmol) in DMF (20 mL) was added HATU (6.50 g, 16 mmol) and N,N-Diisopropylethylamine (4.30 g, 33 mmol) at room temperature. After stirring for 10 min, NH₄Cl (2.90 g, 55 mmol) was added and stirred for 16 h at room temperature. The progress of the reaction was monitored by TLC. After completion of the reaction, mixture was diluted with water (500 mL) and extracted with EtOAc (3×250 mL). The combined organic layer was separated, dried over Na₂SO₄ and concentrated to afford 4-(but-3-yn-1-yl)benzamide (Intermediate 250, 1.5 g) as a white solid, which was used in the next step without further purification. ¹H NMR (400 MHz, DMSO-d₆) δ ppm 2.48-2.50 (m, 2H), 2.77 (s, 1H), 2.80 (t, J=6.8 Hz, 2H), 7.27 (brs, 1H), 7.32 (d, J=8.0 Hz, 2H), 7.79 (d, J=7.6 Hz, 2H), 7.89 (brs, 1H). Mass spec: m/z 174.0 [M+H]⁺.

### Step 5

### Intermediate 251: 4-(4-(2-(2,6-dioxo-1-((2-(trimethylsilyl)ethoxy)methyl)piperidin-3-yl)-1-oxoisoindolin-4-yl)but-3-yn-1-yl)benzamide

To a stirred solution of 4-(but-3-yn-1-yl)benzamide (Intermediate 250, 1 g, 5.77 mmol) and 3-(4-iodo-1-oxoisoindolin-2-yl)-1-((2-(trimethylsilyl)ethoxy)methyl)piperidine-2,6-dione (Intermediate 8, 2.89 g, 5.77 mmol) in DMF (10 mL) was added triethylamine (21.10 g, 207.84 mmol) at room temperature. The reaction mixture was purged with argon gas for 10 min then PdCl₂(PPh₃)₂ (410 mg, 0.58 mmol) and copper (I) iodide (112 mg, 0.58 mmol) was added under argon atmosphere and purging was continued for another 5 min. The reaction mixture was stirred at room temperature for 2 h. The progress of the reaction was monitored by TLC. After completion of the reaction, solvent was evaporated under reduced pressure and the crude material was purified by combi-flash column chromatography, by eluting with 80% EtOAc in heptane, to afford 4-(4-(2-(2,6-dioxo-1-((2-(trimethylsilyl)ethoxy)methyl)piperidin-3-yl)-1-oxoisoindolin-4-yl)but-3-yn-1-yl)benzamide (Intermediate 251, 1.6 g, 51%) as a yellow solid. ¹H NMR (400 MHz, DMSO-d₆) δ ppm −0.009 (s, 9H), 0.82-0.88 (m, 2H), 2.02-2.04 (m, 1H), 2.33-2.37 (m, 1H), 2.81-2.89 (m, 3H), 2.91-3.03 (m, 3H), 3.03-3.11 (m, 1H), 3.51-3.56 (m, 2H), 4.07-4.09 (m, 1H), 4.19-4.21 (m, 1H), 5.04-5.08 (m, 1H), 5.19-5.24 (m, 1H), 7.27 (brs, 1H), 7.38 (d, J=8.0 Hz, 2H), 7.51-7.52 (m, 1H), 7.59 (d, J=7.6 Hz, 1H), 7.70 (d, J=7.2 Hz, 1H), 7.82 (d, J=8.4 Hz, 2H), 7.88 (brs, 1H). Mass spec: m/z 544.0 [M−H]⁻.

### Step 6

### Intermediate 252: tert-butyl 6-(4-(4-(2-(2,6-dioxo-1-((2-(trimethylsilyl)ethoxy)methyl)piperidin×-3-yl)-1-oxoisoindolin-4-yl)but-3-yn-1-yl)benzamido)-2-((R)-1-methylpyrrolidin-2-yl)-1H-pyrrolo[3,2-c]pyridine-1-carboxylate

To a stirred solution of 4-(4-(2-(2,6-dioxo-1-((2-(trimethylsilyl)ethoxy)methyl)piperidin-3-yl)-1-oxoisoindolin-4-yl)but-3-yn-1-yl)benzamide (Intermediate 251, 401 mg, 0.73 mmol) and 1,4-dioxane (10 mL) in was added tert-butyl (R)-6-bromo-2-(1-methylpyrrolidin-2-yl)-1H-pyrrolo[3,2-c]pyridine-1-carboxylate (Intermediate 4, 224 mg, 0.58 mmol) and cesium carbonate (720 mg, 2.21 mmol). After degassing with argon for 15 min, Pd₂(dba)₃ (208 mg, 0.22 mmol) and 4,5-bis(diphenylphosphino)-9,9-dimethylxanthene (66 mg, 0.11 mmol) were added to the reaction mixture and degassed further with argon gas for 10 min. The resulting reaction mixture was heated at 100° C. for 2 h. The progress of the reaction was monitored by TLC. After completion of the reaction, the mixture was directly concentrated under reduced pressure to get crude compound which was purified by combi-flash column chromatography, by eluting with 90% EtOAc in heptane, to afford tert-butyl 6-(4-(4-(2-(2,6-dioxo-1-((2-(trimethylsilyl)ethoxy)methyl)piperidin-3-yl)-1-oxoisoindolin-4-yl)but-3-yn-1-yl)benzamido)-2-((R)-1-methylpyrrolidin-2-yl)-1H-pyrrolo[3,2-c]pyridine-1-carboxylate (Intermediate 252, 390 mg, 63%) as a yellow solid. ¹H NMR (400 MHz, DMSO-d₆) δ ppm −0.06 (s, 9H), 0.78-0.83 (m, 2H), 1.17-1.16 (m, 1H), 1.61-1.66 (m, 2H), 1.69 (s, 9H), 1.75-1.77 (m, 2H), 2.03-2.06 (m, 1H), 2.37 (s, 3H), 2.38-2.41 (m, 1H), 2.76-2.77 (m, 1H), 2.85-2.88 (m, 2H), 2.97-3.06 (m, 3H), 3.13-3.20 (m, 1H), 3.45-3.47 (m, 2H), 3.92-3.94 (m, 1H), 4.07-4.09 (m, 1H), 4.23-4.25 (m, 1H), 4.95-5.01 (m, 2H), 5.17-5.21 (m, 1H), 6.75 (s, 1H), 7.46 (d, J=8.40 Hz, 2H), 7.51 (t, J=7.6 Hz, 1H), 7.61 (d, J=7.6 Hz, 1H), 7.69 (d, J=7.2 Hz, 1H), 8.01 (d, J=7.6 Hz, 2H), 8.58 (s, 1H), 8.93 (s, 1H), 10.59 (s, 1H). Mass spec: m/z 845.2 [M+H]⁺.

### Step 7

### Example 52: 4-(4-(2-(2,6-dioxopiperidin-3-yl)-1-oxoisoindolin-4-yl)but-3-yn-1-yl)-N-(2-((R)-1-methylpyrrolidin-2-yl)-1H-pyrrolo[3,2-c]pyridin-6-yl)benzamide

To a solution of tert-butyl 6-(4-(4-(2-(2,6-dioxo-1-((2-(trimethylsilyl)ethoxy)methyl)piperidin-3-yl)-1-oxoisoindolin-4-yl)but-3-yn-1-yl)benzamido)-2-((R)-1-methylpyrrolidin-2-yl)-1H-pyrrolo[3,2-c]pyridine-1-carboxylate (Intermediate 252, 390 mg, 0.46 mmol) in acetonitrile (8 mL) was added methanesulfonic acid (470 mg, 4.62 mmol) at room temperature and heated to 50° C. for 2 h. After completion of the reaction, mixture was cooled to room temperature and N,N-dimethylethylenediamine (214 mg, 2.31 mmol) and triethylamine (983 mg, 9.23 mmol) were added and stirred at room temperature for 3 h. Progress of reaction was monitored by TLC. After completion of the reaction, solvent was evaporated under reduced pressure, diluted with water (20 mL) to obtain a white precipitate, which was filtered and dried to get the crude compound which was purified by preparative HPLC (Method A) to afford 4-(4-(2-(2,6-dioxopiperidin-3-yl)-1-oxoisoindolin-4-yl)but-3-yn-1-yl)-N-(2-((R)-1-methylpyrrolidin-2-yl)-1H-pyrrolo[3,2-c]pyridin-6-yl)benzamide (Example 52, 110 mg, 39%) as an off white solid. ¹H NMR (400 MHz, DMSO-d₆) δ ppm 1.78-1.99 (m, 3H), 2.01-2.15 (m, 1H), 2.17-2.23 (m, 1H), 2.28 (s, 3H), 2.30-2.35 (m, 1H), 2.41-2.43 (m, 1H), 2.51-2.57 (m, 1H), 2.61-2.81 (m, 3H), 2.84-2.89 (m, 2H), 3.15-3.16 (m, 1H), 3.29-3.33 (m, 1H), 4.26-4.28 (m, 2H), 5.10-5.15 (m, 1H), 6.39 (s, 1H), 7.45-7.59 (m, 2H), 7.60 (d, J=6.80 Hz, 1H), 7.70 (d, J=6.4 Hz, 1H), 7.50 (d, J=8.4 Hz, 2H), 8.18 (s, 1H), 8.19 (s, 1H), 8.49 (s, 1H), 10.33 (s, 1H), 11.12 (s, 1H), 11.36 (s, 1H). Mass spec: m/z 615.1 [M+H]⁺.

### Example 53 was Synthesised Following Scheme 51

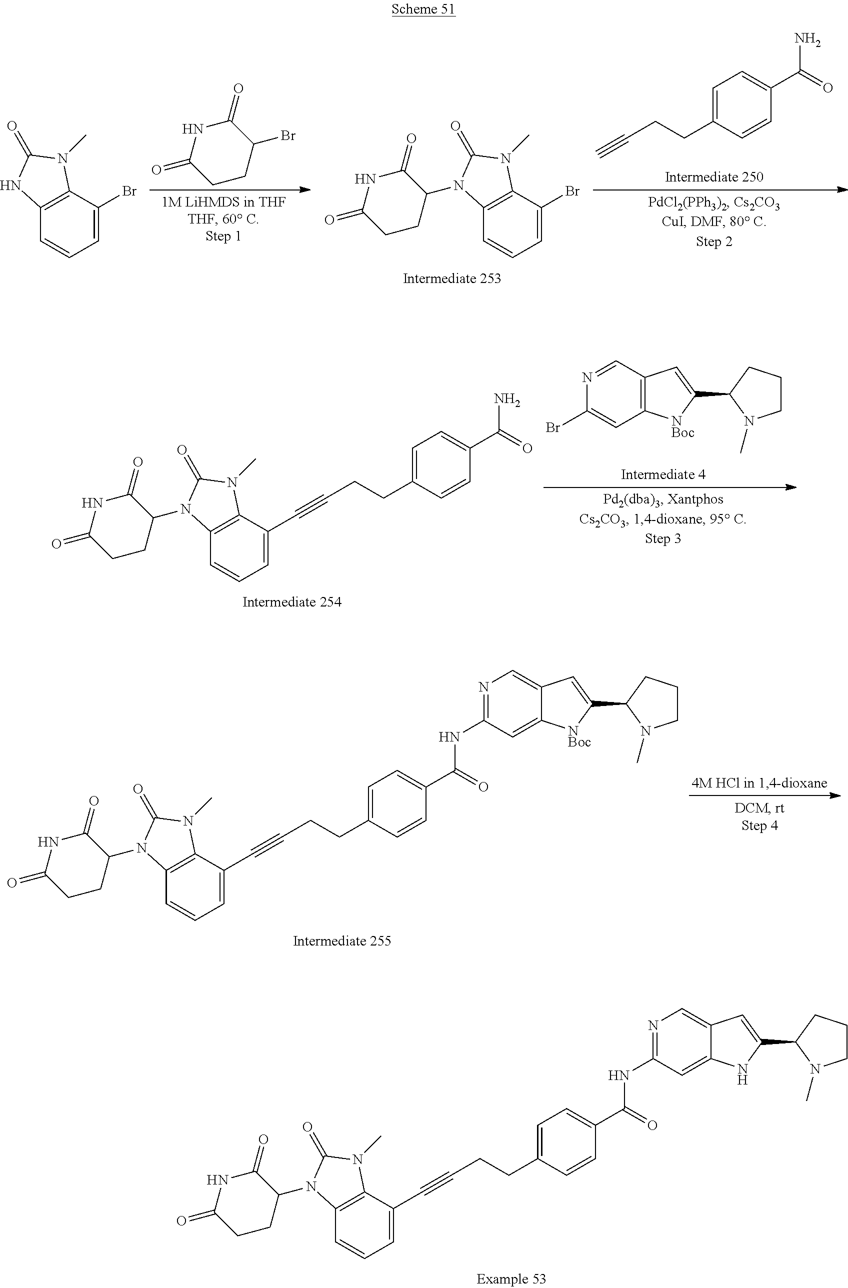

### Step 1

### Intermediate 253: 3-(4-bromo-3-methyl-2-oxo-2,3-dihydro-1H-benzo[d]imidazol-1-yl)piperidine-2,6-dione

To a solution of 7-bromo-1-methyl-1,3-dihydro-2H-benzo[d]imidazol-2-one (CAS No: 913297-44-6, 5.00 g, 22 mmol) in tetrahydrofuran (20 mL) was added 1M LiHMDS in THF (55 mL, 55 mmol). The resulting solution was then added dropwise to 3-bromopiperidine-2,6-dione (CAS No: 62595-74-8, 8.5 g, 44 mmol) in THF (55 mL) at room temperature and the reaction mixture was stirred at 60° C. for 4 h. The reaction mixture was poured into saturated aqueous NH₄Cl solution (250 mL) and stirred for 10 min at room temperature. The resulting solid was collected by filtration, washed with ethyl acetate (20 mL) and dried under vacuum to afford 3-(4-bromo-3-methyl-2-oxo-2,3-dihydro-1H-benzo[d]imidazol-1-yl)piperidine-2,6-dione (Intermediate 253, 4.00 g, 54%) as a grey solid, which was used in the next step without further purification. ¹H NMR (400 MHz, DMSO-d₆) δ ppm 1.98-2.08 (m, 1H), 2.62-2.73 (m, 2H), 2.83-2.95 (m, 1H), 3.64 (s, 3H), 5.39-5.43 (m, 1H), 6.94-7.02 (m, 1H), 7.17 (d, J=8.20 Hz, 1H), 7.24 (d, J=8.20 Hz, 1H), 11.12 (br s, 1H). Mass spec: m/z: 338.04 [M+H]⁺.

### Step 2

### Intermediate 254: 4-(4-(1-(2,6-dioxopiperidin-3-yl)-3-methyl-2-oxo-2,3-dihydro-1H-benzo[d]imidazol-4-yl) but-3-yn-1-yl)benzamide

To a solution of 4-(but-3-yn-1-yl)benzamide (Intermediate 250, 0.49 g, 2.88 mmol) in dimethylformamide (15 mL) was added 3-(4-bromo-3-methyl-2-oxo-2,3-dihydro-1H-benzo[d]imidazol-1-yl)piperidine-2,6-dione (Intermediate 253, 0.65 g, 1.92 mmol) followed by cesium carbonate (1.98 g, 5.76 mmol) and the reaction mixture was purged with argon for 15 min. Copper(I) iodide (0.037 g, 0.19 mmol) and PdCl₂(PPh₃)₂ (0.14 g, 0.19 mmol) were then added and the reaction mixture was further purged with argon for 10 min and then stirred at 80° C. for 3 h. The reaction mixture was concentrated in vacuo to obtain crude compound. The crude material was purified by combi-flash column chromatography by eluting with 15% MeOH in DCM to afford 4-(4-(1-(2,6-dioxopiperidin-3-yl)-3-methyl-2-oxo-2,3-dihydro-1H-benzo[d]imidazol-4-yl)but-3-yn-1-yl)benzamide (Intermediate 254, 0.40 g, 48%) as a brown solid. ¹H NMR (400 MHz, DMSO-d₆) δ ppm 2.58-2.74 (m, 2H), 2.77-2.99 (m, 6H), 3.38 (s, 3H), 5.34-5.38 (m, 1H), 6.92-7.13 (m, 2H), 7.24-7.46 (m, 3H), 7.81-7.92 (m, 3H), 11.09 (br s, 1H). Mass spec: m/z: 429.26 [M−H]⁻.

### Step 3

### Intermediate 255: tert-butyl 6-(4-(4-(1-(2,6-dioxopiperidin-3-yl)-3-methyl-2-oxo-2,3-dihydro-1H-benzo[d]imidazol-4-yl)but-3-yn-1-yl)benzamido)-2-((R)-1-methylpyrrolidin-2-yl)-1H-pyrrolo[3,2-c]pyridine-1-carboxylate

To a solution of 4-(4-(1-(2,6-dioxopiperidin-3-yl)-3-methyl-2-oxo-2,3-dihydro-1H-benzo[d]imidazol-4-yl)but-3-yn-1-yl)benzamide (Intermediate 254, 0.38 g, 0.88 mmol) in 1,4-dioxane (9 mL) was added tert-butyl (R)-6-bromo-2-(1-methylpyrrolidin-2-yl)-1H-pyrrolo[3,2-c]pyridine-1-carboxylate (Intermediate 4, 0.40 g, 1.06 mmol) followed by cesium carbonate (0.87 g, 2.65 mmol) at room temperature. The reaction mixture was purged with argon for 15 min then Pd₂(dba)₃ (0.11 g, 0.13 mmol) and Xantphos (0.16 g, 0.26 mmol) were added and the reaction mixture was further purged with argon for 10 min and stirred at 95° C. for 1 h. The reaction mixture was filtered through celite, washed with dichloromethane (20 mL) and the filtrate was concentrated in vacuo to obtain crude compound. The crude material was purified by combi-flash column chromatography, by eluting with 10% MeOH in DCM, to afford tert-butyl 6-(4-(4-(1-(2,6-dioxopiperidin-3-yl)-3-methyl-2-oxo-2,3-dihydro-1H-benzo[d]imidazol-4-yl)but-3-yn-1-yl)benzamido)-2-((R)-1-methylpyrrolidin-2-yl)-1H-pyrrolo[3,2-c]pyridine-1-carboxylate (Intermediate 255, 0.40 g) as a yellow solid, which was used for the next step without further purification. Mass spec: m/z: 730.1 [M+H]⁺.

### Step 4

### Example 53: 4-(4-(1-(2,6-dioxopiperidin-3-yl)-3-methyl-2-oxo-2,3-dihydro-1H-benzo[d]imidazol-4-yl)but-3-yn-1-yl)-N-(2-((R)-1-methylpyrrolidin-2-yl)-1H-pyrrolo[3,2-c]pyridin-6-yl)benzamide

To a solution of tert-butyl 6-(4-(4-(1-(2,6-dioxopiperidin-3-yl)-3-methyl-2-oxo-2,3-dihydro-1H-benzo[d]imidazol-4-yl)but-3-yn-1-yl)benzamido)-2-((R)-1-methylpyrrolidin-2-yl)-1H-pyrrolo[3,2-c]pyridine-1-carboxylate (Intermediate 255, 0.38 g, 0.52 mmol) in dichloromethane (3.0 mL) was added 4M hydrochloric acid in 1,4-dioxane (0.3 mL, 1.0 mmol) at 0° C. The reaction mixture was then stirred at room temperature for 2 h. The reaction mixture was concentrated in vacuo and crude residue was washed with diethyl ether (10 mL) and dried under vacuum. This material was then purified by preparative HPLC (Method A) to afford 4-(4-(1-(2,6-dioxopiperidin-3-yl)-3-methyl-2-oxo-2,3-dihydro-1H-benzo[d]imidazol-4-yl)but-3-yn-1-yl)-N-(2-((R)-1-methylpyrrolidin-2-yl)-1H-pyrrolo[3,2-c]pyridin-6-yl)benzamide (Example 53, 0.01 g, 3%) as an off white solid. ¹H NMR (400 MHz, DMSO-d₆) δ ppm 1.75-1.95 (m, 3H), 1.95-2.05 (m, 1H), 2.12-2.15 (m, 1H), 2.17 (s, 3H), 2.22-2.30 (m, 1H), 2.57-2.67 (m, 3H), 2.85-2.93 (m, 3H), 2.94-3.03 (m, 2H), 3.11-3.18 (m, 1H), 3.43 (s, 3H), 5.33-5.38 (m, 1H), 6.39 (s, 1H), 6.94-7.05 (m, 2H), 7.08-7.10 (m, 1H), 7.46 (d, J=8.25 Hz, 2H), 8.02 (d, J=8.25 Hz, 2H), 8.19. Mass spec: m/z: 630.0[M+H]⁺.

### Example 54 was Synthesised Following Scheme 52

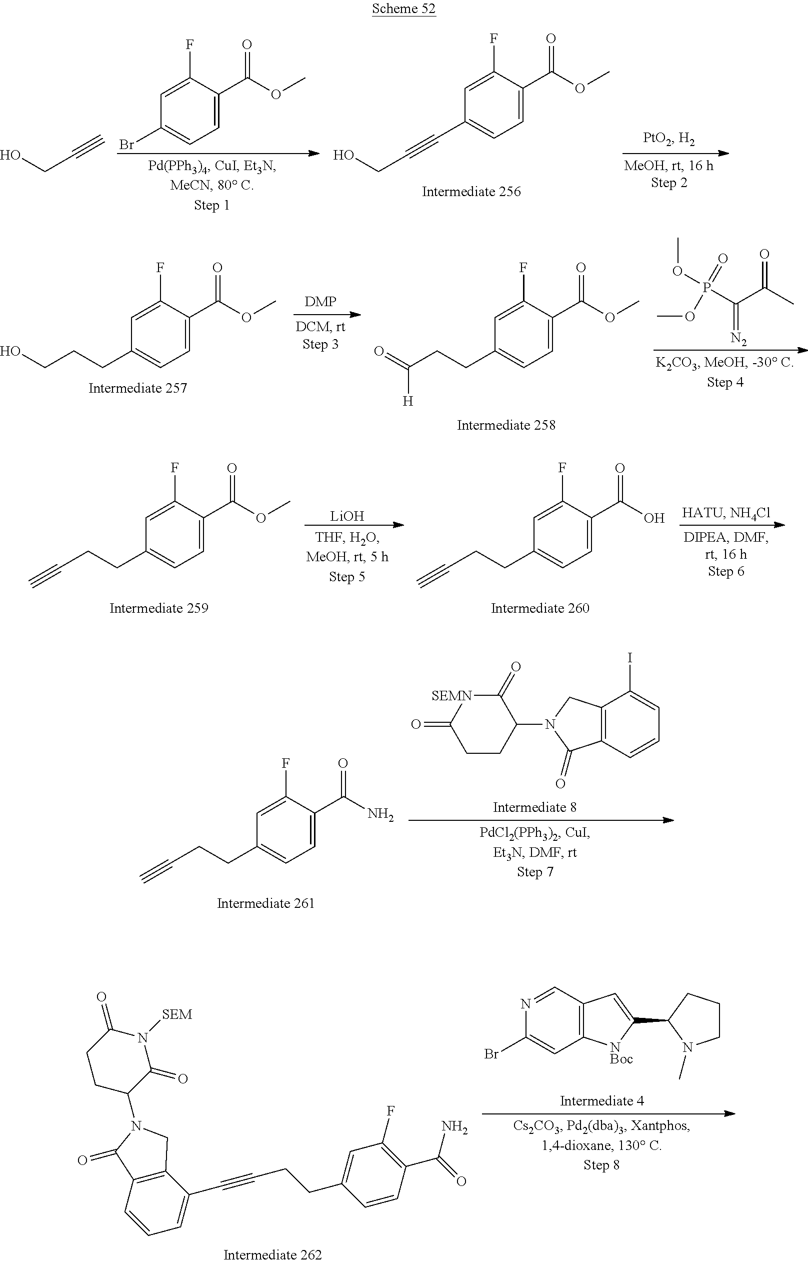

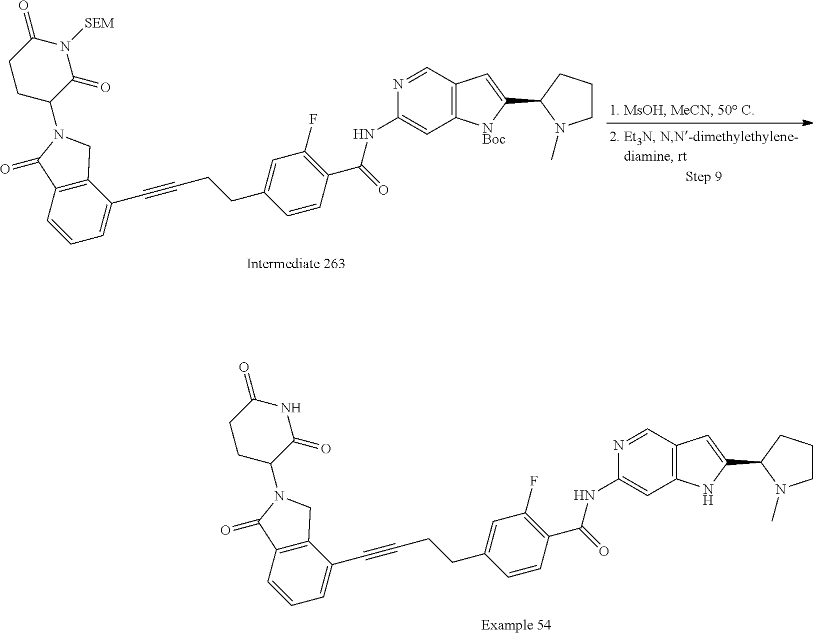 [SMILES: CN1CCC[C@@H]1c1cc2cnc(NC(=O)c3ccc(CCC#Cc4cccc5c4CN(C4CCC(=O)N(COCC[Si](C)(C)C)C4=O)C5=O)cc3F)cc2n1C(=O)OC(C)(C)C ; CN1CCC[C@@H]1c1cc2cnc(NC(=O)c3ccc(CCC#Cc4cccc5c4CN(C4CCC(=O)NC4=O)C5=O)cc3F)cc2[nH]1]

### Step 1

### Intermediate 256: methyl 2-fluoro-4-(3-hydroxyprop-1-yn-1-yl)benzoate

To a solution of methyl 4-bromo-2-fluorobenzoate (CAS No. 179232-29-2, 25.0 g, 107.28 mmol) in acetonitrile (250 mL) were added copper(I) iodide (2.08 g, 10.72 mmol), PdCl₂(PPh₃)₂ (7.60 g, 10.72 mmol), prop-2-yn-1-ol (CAS No. 107-19-7, 12.03 g, 214.56 mmol) and triethylamine (60.1 mL, 429.13 mmol). The reaction mixture was purged with argon for 15 min. The reaction mixture was heated at 80° C. for 16 h. Progress of the reaction was monitored by TLC. After completion, the reaction mixture was concentrated in vacuo. The reaction mixture was diluted with aqueous saturated sodium thiosulphate solution (200 mL) and extracted with EtOAc (2×200 mL). The organic layer was separated, dried over anhydrous Na₂SO₄ and concentrated in vacuo to obtain crude material. The crude material was purified by combi-flash column chromatography, by eluting with 30% EtOAc in heptane, to afford methyl 2-fluoro-4-(3-hydroxyprop-1-yn-1-yl)benzoate (Intermediate 256, 18.0 g, 81%) as a yellow solid. ¹H NMR (400 MHz, DMSO-d₆) δ ppm 3.82 (s, 3H), 4.30 (d, J=6.14 Hz, 2H), 5.40 (t, J=5.92 Hz, 1H), 7.30-7.40 (m, 2H), 7.83-7.84 (m, 1H).

### Step 2

### Intermediate 257: methyl 2-fluoro-4-(3-hydroxypropyl)benzoate

To a solution methyl 2-fluoro-4-(3-hydroxyprop-1-yn-1-yl)benzoate (Intermediate 256, 17.0 g, 81.66 mmol) in methanol (400 mL) was added platinum oxide (1.85 g, 8.16 mmol). The reaction mixture was stirred at room temperature for 16 h under H₂ atmosphere at 50 psi.

Progress of the reaction was monitored by TLC. After completion of reaction, the reaction mixture was filtered through a celite bed and washed with MeOH (100 mL). The solvent was evaporated under reduced pressure to obtain the crude material. This material was purified by combi-flash column chromatography, by eluting with 30% EtOAc in heptane, to afford methyl 2-fluoro-4-(3-hydroxypropyl)benzoate (Intermediate 257, 10.50 g, 61%) as a yellow liquid. ¹H NMR (400 MHz, DMSO-d₆) δ ppm 1.70-1.76 (m, 2H), 2.68 (t, J=7.45 Hz, 2H), 3.38-2.42 (m, 2H), 3.83 (s, 3H), 4.50 (t, J=4.60 Hz, 1H), 7.15-7.20 (m, 2H), 7.80-7.81 (m, 1H). Mass spec: m/z 213.0 [M+H]⁺.

### Step 3

### Intermediate 258: methyl 2-fluoro-4-(3-oxopropyl)benzoate

To a solution of methyl 2-fluoro-4-(3-hydroxypropyl)benzoate (Intermediate 257, 10.0 g, 47.1 mmol) in dichloromethane (100 mL) was added Dess-Martin periodinane (30.0 g, 70.7 mmol) at 0° C. The reaction mixture was stirred at room temperature for 2 h. Progress of the reaction was monitored by TLC. After completion, the reaction mixture filtered through a celite bed. The reaction mixture was quenched with NaHCO₃ solution (200 mL) and extracted with DCM (3×200 mL). The organic layer was separated, dried over anhydrous Na₂SO₄ and concentrated in vacuo. The crude material was purified by combi-flash column chromatography, by eluting with 30% EtOAc in heptane, to afford methyl 2-fluoro-4-(3-oxopropyl)benzoate (Intermediate 258, 8.5 g, 85%) as a yellow liquid. ¹H NMR (400 MHz, DMSO-d₆) δ ppm 2.81-2.88 (m, 4H), 3.80 (s, 3H), 7.15-7.22 (m, 2H), 7.76-7.77 (m, 1H), 9.67 (s, 1H).

### Step 4

### Intermediate 259: methyl 4-(but-3-yn-1-yl)-2-fluorobenzoate

To a solution of methyl 2-fluoro-4-(3-oxopropyl)benzoate (Intermediate 258, 8.5 g, 35.7 mmol) in methanol (100 mL) was added potassium carbonate (6.7 g, 49 mmol) at −30° C. and the reaction mixture was stirred for 10 min, to this mixture dimethyl (1-diazo-2-oxopropyl) phosphonate (13.0 g, 61.0 mmol) was added dropwise at −30° and the reaction mixture was stirred at same temperature for 3 h. Progress of the reaction was monitored by TLC. After completion, the reaction mixture was diluted with water (100 mL) and extracted with EtOAc (3×150 mL). The combined organic layer was separated, dried over anhydrous Na₂SO₄ and concentrated in vacuo. The crude material was purified by combi-flash column chromatography, by eluting with 10% EtOAc in heptane, to afford methyl 4-(but-3-yn-1-yl)-2-fluorobenzoate (Intermediate 259, 5.70 g, 68%) as a yellow liquid. ¹H NMR (400 MHz, DMSO-d₆) δ ppm 2.49-2.53 (m, 2H), 2.79-2.83 (m, 2H), 2.85 (s, 1H), 3.84 (s, 3H), 7.21-7.28 (m, 2H), 7.81-7.82 (m, 1H).

### Step 5

### Intermediate 260: 4-(but-3-yn-1-yl)-2-fluorobenzoic acid

To a solution of methyl 4-(but-3-yn-1-yl)-2-fluorobenzoate (Intermediate 259, 5.70 g, 27.6 mmol) in tetrahydrofuran (30 mL) and methanol (30 mL) was added lithium hydroxide (2.03 g, 82.9 mmol) in water (15 mL) and the reaction mixture was stirred at room temperature for 5 h. Progress of the reaction was monitored by TLC. After completion, the reaction mixture was concentrated in vacuo. The reaction mixture was diluted with water (10 mL), the reaction mixture was acidified to pH~2 by adding citric acid solution to obtain a white solid. This was filtered and dried to afford 4-(but-3-yn-1-yl)-2-fluorobenzoic acid (Intermediate 260, 4.7 g, 88%) as a white solid. ¹H NMR (400 MHz, DMSO-d₆) δ ppm 2.51-2.54 (m, 2H), 2.78-2.82 (m, 2H), 2.84 (s, 1H), 7.17-7.23 (m, 2H), 7.78-7.79 (m, 1H), 13.07 (br s, 1H). Mass spec: m/z 190.9 [M−H]⁺.

### Step 6

### Intermediate 261: 4-(but-3-yn-1-yl)-2-fluorobenzamide

To a solution of 4-(but-3-yn-1-yl)-2-fluorobenzoic acid (Intermediate 260, 4.7 g, 24 mmol) in DMF (50 mL) was added diisopropylethylamine (9.5 g, 73 mmol) and HATU (14.0 g, 37 mmol) followed by ammonium chloride (6.6 g, 120 mmol). The reaction mixture was stirred at room temperature for 16 h. Progress of the reaction was monitored by TLC. After completion, the reaction mixture was poured in ice cold water (200 mL) and extracted with EtOAc (3×100 mL). The organic layer was separated, dried over anhydrous Na₂SO₄ and concentrated in vacuo to afford 4-(but-3-yn-1-yl)-2-fluorobenzamide (Intermediate 261, 3.5 g, 75%) as a white solid which was used for the next step without further purification. Mass spec: m/z 192.0 [M+H]⁺.

### Step 7

### Intermediate 262: 4-(4-(2-(2,6-dioxo-1-((2-(trimethylsilyl) ethoxy)methyl) piperidin-3-yl)-1-oxoisoindolin-4-yl) but-3-yn-1-yl)-2-fluorobenzamide

To a solution of 4-(but-3-yn-1-yl)-2-fluorobenzamide (Intermediate 261, 1.0 g, 5.2 mmol) in DMF (10 mL) was added copper(I) iodide (0.10 g, 0.52 mmol), PdCl₂(PPh₃)₂ (0.37 g, 0.52 mmol), 3-(4-iodo-1-oxoisoindolin-2-yl)-1-((2-(trimethylsilyl)ethoxy)methyl)piperidine-2,6-dione (Intermediate 8, 2.6 g, 5.2 mmol) and triethylamine (26 mL, 180 mmol). The reaction mixture was purged with argon for 15 min. The reaction mixture was stirred at room temperature for 2 h. Progress of the reaction was monitored by TLC. After completion of the reaction, aqueous saturated sodium thiosulphate solution (100 mL) was added and extracted with EtOAc (2×100 mL). The organic layer was separated, dried over anhydrous Na₂SO₄ and concentrated in vacuo. The crude material was purified by combi-flash column chromatography, by eluting with 70% EtOAc in heptane, to afford 4-(4-(2-(2,6-dioxo-1-((2-(trimethylsilyl)ethoxy)methyl)piperidin-3-yl)-1-oxoisoindolin-4-yl)but-3-yn-1-yl)-2-fluorobenzamide (Intermediate 262, 1.20 g, 41%) as a yellow solid. ¹H NMR (400 MHz, DMSO-d₆) δ ppm −0.03 (s, 9H), 0.84-0.88 (m, 2H), 1.98-2.06 (m, 1H), 2.36-2.40 (m, 1H), 2.77-2.87 (m, 4H), 2.87-2.96 (m, 2H), 3.50-3.56 (m, 2H), 4.10-4.15 (m, 1H), 4.25-4.30 (m, 1H), 5.03-5.10 (m, 2H), 5.21-5.26 (m, 1H), 7.20-7.30 (m, 3H), 7.48-7.61 (m, 3H), 7.71 (br s, 1H), 7.95 (br s, 1H). Mass spec: m/z 562.1 [M−H]⁺.

### Step 8

### Intermediate 263: tert-butyl 6-(4-(4-(2-(2,6-dioxo-1-((2-(trimethylsilyl)ethoxy)methyl)piperidin-3-yl)-1-oxoisoindolin-4-yl)but-3-yn-1-yl)-2-fluorobenzamido)-2-((R)-1-methylpyrrolidin-2-yl)-1H-pyrrolo[3,2-c]pyridine-1-carboxylate

To a solution of 4-(4-(2-(2,6-dioxo-1-((2-(trimethylsilyl) ethoxy)methyl)piperidin-3-yl)-1-oxoisoindolin-4-yl) but-3-yn-1-yl)-2-fluorobenzamide (Intermediate 262, 0.6 g, 1.0 mmol) in 1,4-dioxane (20 mL) was added tert-butyl (R)-6-bromo-2-(1-methylpyrrolidin-2-yl)-1H-pyrrolo[3,2-c]pyridine-1-carboxylate (Intermediate 4, 0.5 g, 1.0 mmol) followed by cesium carbonate (1.0 g, 3.0 mmol). The reaction mixture was purged with argon for 10 min. then added Pd₂(dba)₃ (0.2 g, 0.2 mmol) and xantphos (0.2 g, 0.3 mmol) at room temperature. The reaction mixture was purged with argon for 10 min and heated at 130° C. for 2 h. Progress of the reaction was monitored by TLC. After completion, the reaction mixture was concentrated in vacuo. The crude material was purified by combi-flash column chromatography, by eluting with 80% EtOAc in heptane, to afford tert-butyl 6-(4-(4-(2-(2,6-dioxo-1-((2-(trimethylsilyl)ethoxy)methyl)piperidin-3-yl)-1-oxoisoindolin-4-yl)but-3-yn-1-yl)-2-fluorobenzamido)-2-((R)-1-methylpyrrolidin-2-yl)-1H-pyrrolo[3,2-c]pyridine-1-carboxylate (Intermediate 263, 0.3 g, 30%) as a yellow solid. ¹H NMR (400 MHz, DMSO-d₆) δ ppm −0.07 (s, 9H), 0.73-0.79 (m, 2H), 1.16-1.19 (m, 1H), 1.59-1.65 (m, 3H), 1.70 (s, 9H), 2.35 (s, 3H), 2.72-2.76 (m, 2H), 2.83-2.89 (m, 4H), 2.93-3.01 (m, 4H), 3.42-3.46 (m, 2H), 3.90-3.94 (m, 1H), 4.11-4.15 (m, 1H), 4.31-4.36 (m, 1H), 4.94-5.04 (m, 2H), 5.21-5.26 (m, 1H), 6.74 (s, 1H), 7.27-7.38 (m, 2H), 7.49-7.54 (m, 2H), 7.61-7.72 (m, 2H), 8.55 (s, 1H), 8.92 (s, 1H), 10.56 (br s, 1H). Mass spec: m/z 863.1 [M+H]⁺.

### Step 9

### Example 54: 4-(4-(2-(2,6-dioxopiperidin-3-yl)-1-oxoisoindolin-4-yl) but-3-yn-1-yl)-2-fluoro-N-(2-((R)-1-methylpyrrolidin-2-yl)-1H-pyrrolo[3,2-c]pyridin-6-yl)benzamide

To a solution of tert-butyl 6-(4-(4-(2-(2,6-dioxo-1-((2-(trimethylsilyl)ethoxy)methyl)piperidin-3-yl)-1-oxoisoindolin-4-yl)but-3-yn-1-yl)-2-fluorobenzamido)-2-((R)-1-methylpyrrolidin-2-yl)-1H-pyrrolo[3,2-c]pyridine-1-carboxylate (Intermediate 263, 0.5 g, 0.6 mmol) in acetonitrile (8 mL) was added methanesulfonic acid (0.6 mL, 9.0 mmol) at room temperature and the reaction mixture was heated at 50° C. for 2 h. To the reaction mixture triethylamine (2.0 mL, 10 mmol) and N,N-dimethylethylenediamine (0.4 mL, 3.0 mmol) were added at room temperature and stirred for 3 h. Progress of the reaction was monitored by TLC. After completion, the reaction mixture was poured into ice cold water, a white solid precipitated, which was filtered and dried to obtain crude material. The crude was purified by preparative HPLC (Method A) to afford 4-(4-(2-(2,6-dioxopiperidin-3-yl)-1-oxoisoindolin-4-yl)but-3-yn-1-yl)-2-fluoro-N-(2-((R)-1-methylpyrrolidin-2-yl)-1H-pyrrolo[3,2-c]pyridin-6-yl)benzamide (Example 54, 0.1 g, 30%) as an off white solid. ¹H NMR (400 MHz, DMSO-d₆) δ ppm 1.77-1.90 (m, 3H), 1.99-2.03 (m, 1H), 2.10-2.14 (m, 1H), 2.16 (s, 3H), 2.23-2.29 (m, 1H), 2.39-2.46 (m, 1H), 2.56-2.60 (m, 1H), 2.86-2.91 (m, 3H), 2.93-3.02 (m, 2H), 3.12-3.16 (m, 1H), 3.28-3.30 (m, 1H), 4.21-4.26 (m, 1H), 4.32-4.36 (m, 1H), 5.11-5.16 (m, 1H), 6.40 (s, 1H), 7.30-7.31 (m, 1H), 7.35-7.36 (m, 1H), 7.50-7.53 (m, 1H), 7.61-7.62 (m, 1H), 7.69-7.73 (m, 2H), 8.19 (s, 1H), 8.46 (s, 1H), 10.23 (s, 1H), 11.14 (s, 1H), 11.40 (s, 1H). Mass spec: m/z 633.2 [M+H]⁺.

### Example 55 was Synthesised Following Scheme 53

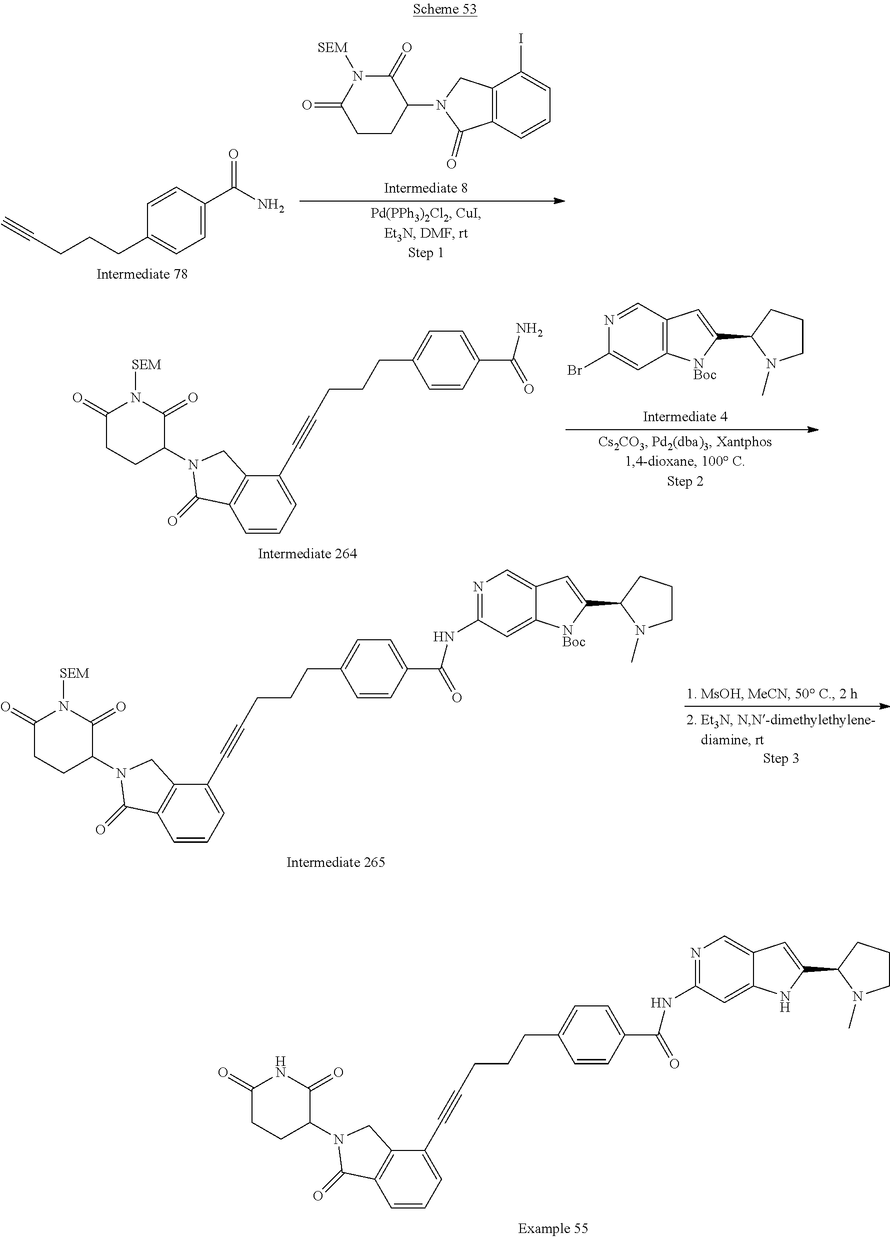 [SMILES: C#CCCCc1ccc(C(N)=O)cc1 ; C[Si](C)(C)CCOCN1C(=O)CCC(N2Cc3c(I)cccc3C2=O)C1=O ; C[Si](C)(C)CCOCN1C(=O)CCC(N2Cc3c(C#CCCCc4ccc(C(N)=O)cc4)cccc3C2=O)C1=O ; CN1CCC[C@@H]1c1cc2cnc(Br)cc2n1C(=O)OC(C)(C)C ; CN1CCC[C@@H]1c1cc2cnc(NC(=O)c3ccc(CCCC#Cc4cccc5c4CN(C4CCC(=O)N(COCC[Si](C)(C)C)C4=O)C5=O)cc3)cc2n1C(=O)OC(C)(C)C ; CN1CCC[C@@H]1c1cc2cnc(NC(=O)c3ccc(CCCC#Cc4cccc5c4CN(C4CCC(=O)NC4=O)C5=O)cc3)cc2[nH]1]

### Step 1

### Intermediate 264: 4-(5-(2-(2,6-dioxo-1-((2-(trimethylsilyl)ethoxy)methyl)piperidin-3-yl)-1-oxoisoindolin-4-yl)pent-4-yn-1-yl)benzamide

To a solution of 4-(pent-4-yn-1-yl)benzamide (Intermediate 78, 1 g, 5.34 mmol) in DMF (15 mL), 3-(4-iodo-1-oxoisoindolin-2-yl)-1-((2-(trimethylsilyl)ethoxy)methyl)piperidine-2,6-dione (Intermediate 8, 2.67 g, 5.37 mmol), triethylamine (20.1 g, 197.61 mmol), CuI (0.1 g, 0.534 mmol) and PdCl₂(PPh₃)₂ (0.38 g, 5.37 mmol) were added and the argon has was passed through the reaction mixture for 15 min. The reaction mixture was stirred at room temperature for 16 h. Progress of reaction was monitored by TLC. After completion of reaction, the mixture was concentrated under reduced pressure to obtain the crude material which was purified by combi-flash column chromatography by eluting with 80% EtOAc in heptane to afford 4-(5-(2-(2,6-dioxo-1-((2-(trimethylsilyl)ethoxy)methyl)piperidin-3-yl)-1-oxoisoindolin-4-yl)pent-4-yn-1-yl)benzamide (Intermediate 264, 2.3 g, 77%) as green solid. ¹H NMR (400 MHz, DMSO-d₆) δ ppm −0.08 (s, 9H), 2.43-2.45 (m, 2H), 2.70 (s, 1H), 2.73-2.78 (m, 3H), 2.86 (s, 1H), 3.25-3.26 (m, 1H), 3.25-3.29 (m, 4H), 4.21-4.29 (m, 1H), 4.42-4.50 (m, 1H), 4.96-5.08 (m, 2H), 5.24 (dd, J=13.15, 4.38 Hz, 1H), 7.22 (br s, 1H), 7.22-7.27 (m, 2H), 7.47-7.54 (m, 1H), 7.60-7.72 (m, 2H), 7.77-7.80 (m, 2H), 7.84 (br s, 1H). Mass spec: m/z 558.1 [M+H]⁺.

### Step 2

### Intermediate 265: tert-butyl 6-(4-(5-(2-(2,6-dioxo-1-((2-(trimethylsilyl)ethoxy)methyl)piperidin-3-yl)-1-oxoisoindolin-4-yl)pent-4-yn-1-yl)benzamido)-2-((R)-1-methylpyrrolidin-2-yl)-1H-pyrrolo[3,2-c]pyridine-1-carboxylate

To a solution of tert-butyl (R)-6-bromo-2-(1-methylpyrrolidin-2-yl)-1H-pyrrolo[3,2-c]pyridine-1-carboxylate (Intermediate 4, 1.0 g, 2.60 mmol) in 1,4-dioxane (20 mL) was added cesium carbonate (2.60 g, 7.90 mmol) and 4-(5-(2-(2,6-dioxo-1-((2-(trimethylsilyl)ethoxy)methyl)piperidin-3-yl)-1-oxoisoindolin-4-yl)pent-4-yn-1-yl)benzamide (Intermediate 264, 1.16 g, 2.08 mmol). The reaction mixture was degassed with argon gas for 10 min then added xantphos (0.47 g, 0.79 mmol) and Pd₂(dba)₃ (0.37 g, 0.39 mmol) and the reaction mixture was degassed with argon gas for 10 min and heated at 130° C. for 2 h. Progress of reaction was monitored by TLC. After completion of reaction, the reaction mixture was concentrated under reduced pressure to obtain the crude compound which was purified by combi-flash column chromatography by eluting with 80% EtOAc in heptane to afford tert-butyl 6-(4-(5-(2-(2,6-dioxo-1-((2-(trimethylsilyl)ethoxy)methyl)piperidin-3-yl)-1-oxoisoindolin-4-yl)pent-4-yn-1-yl)benzamido)-2-((R)-1-methylpyrrolidin-2-yl)-1H-pyrrolo[3,2-c]pyridine-1-carboxylate (Intermediate 265, 0.55 g, 24%) as a yellow solid. ¹H NMR (400 MHz, DMSO-d₆) δ ppm −0.05 (s, 9H), 0.70-0.81 (m, 2H), 1.54-1.65 (m, 3H), 1.68 (s, 9H), 1.69-1.70 (m, 2H), 1.92-2.07 (m, 3H), 2.33 (s, 3H), 2.34-2.36 (m, 2H), 2.74-2.88 (m, 3H), 3.01-3.06 (m, 3H), 3.51 (m, 2H), 3.92 (m, 1H), 4.27-4.92 (m, 2H), 5.01-5.08 (m, 2H), 5.26-5.28 (m, 1H), 6.73-6.74 (m, 1H), 7.36-7.38 (m, 2H), 7.52-7.54 (m, 1H), 7.67-7.73 (m, 2H), 7.98-8.00 (m, 2H), 8.58 (s, 1H), 8.92 (s, 1H), 10.58 (s, 1H). Mass spec: m/z 859.2 [M+H]⁺.

### Step 3

### Example 55: 4-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide

To a stirred solution of tert-butyl 6-[[4-[5-[2-[2,6-dioxo-1-(2-trimethylsilylethoxymethyl)-3-piperidyl]-1-oxo-isoindolin-4-yl]pent-4-ynyl]benzoyl]amino]-2-[(2-(R))-1-methylpyrrolidin-2-yl]pyrrolo[3,2-c]pyridine-1-carboxylate (Intermediate 265, 0.5 g, 0.58 mmol) in acetonitrile (10 mL) was added methanesulfonic acid (0.85 g, 8.73 mmol) at room temperature. The reaction mixture was heated at 50° C. for 2 h then reaction mixture was allowed to cool to room and N—N′-dimethylethylenediamine (0.34 g, 3.49 mmol) and triethylamine (0.01 g, 11.64 mmol) were added. The reaction was stirred at room temperature for 3 h and monitored by TLC. After completion of reaction, the mixture was concentrated under reduced pressure to get crude material which was purified by preparative HPLC (Method A) to afford 4-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide (Example 55, 0.085 g, 23%) as off-white solid. ¹H NMR (400 MHz, DMSO-d₆) δ ppm 1.77-2.05 (m, 5H), 2.11-2.15 (m, 1H), 2.17 (s, 3H), 2.21-2.32 (m, 1H), 2.35-2.47 (m, 2H), 2.53-2.64 (m, 3H), 2.77-2.96 (m, 3H), 3.07-3.22 (m, 1H), 3.33-3.34 (m, 1H), 4.36-4.51 (m, 2H), 5.12-5.17 (m, 1H), 6.39 (s, 1H), 7.37-7.39 (m, 2H), 7.53-7.55 (m, 1H), 7.63-7.75 (m, 2H), 8.00-8.02 (m, 2H), 8.19 (s, 1H), 8.50 (s, 1H), 10.34 (s, 1H) 11.02 (s, 1H), 11.36 (s, 1H). Mass spec: m/z 629.0 [M+H]⁺.

### Example 56 was Synthesised Following Scheme 54

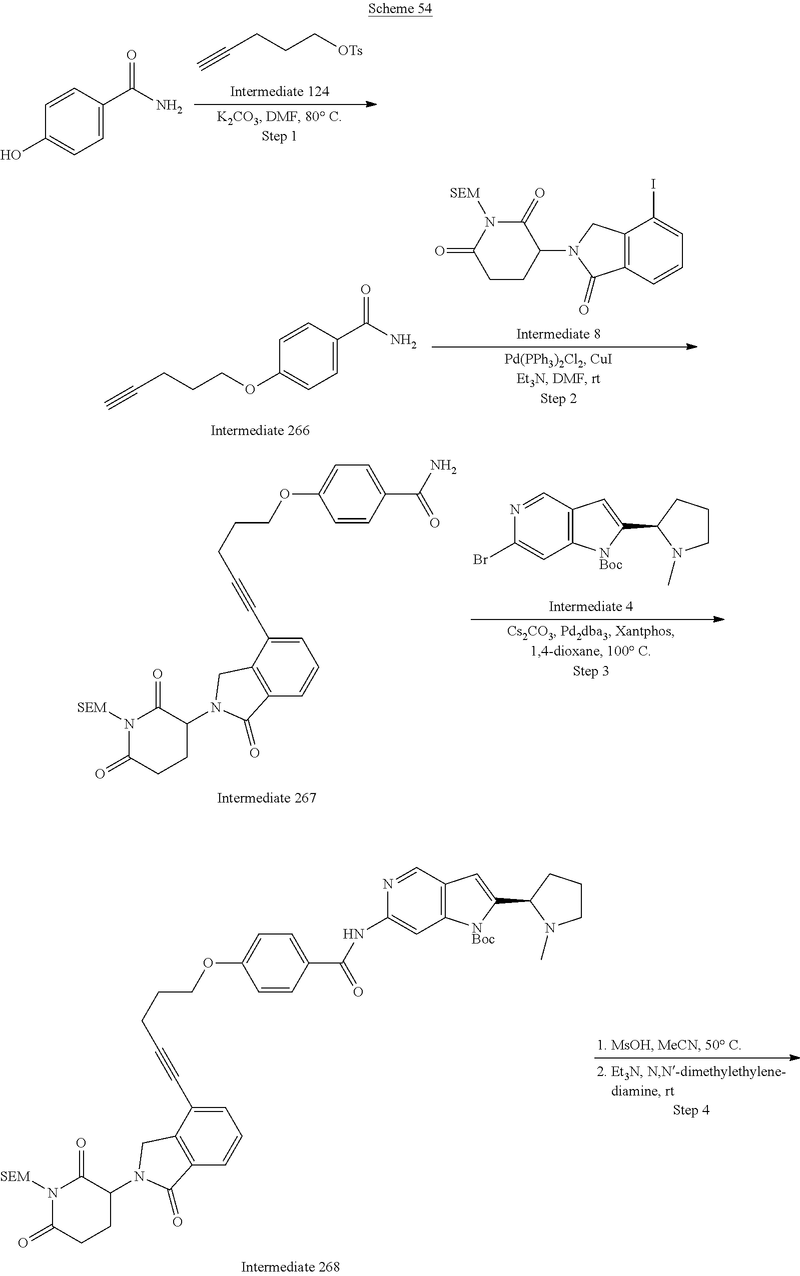

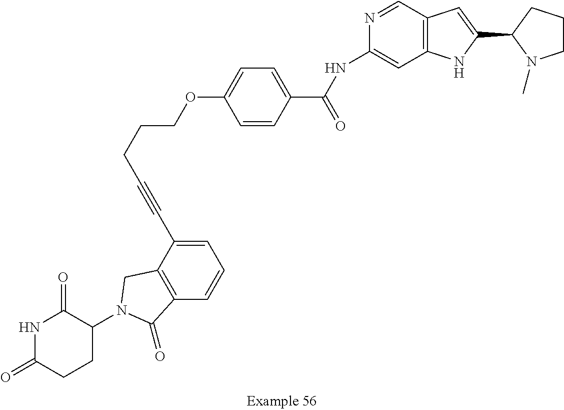 [SMILES: CN1CCC[C@@H]1c1cc2cnc(NC(=O)c3ccc(OCCCC#Cc4cccc5c4CN(C4CCC(=O)NC4=O)C5=O)cc3)cc2[nH]1]

### Step 1

### Intermediate 266: 4-(pent-4-yn-1-yloxy)benzamide

To a solution of 4-hydroxybenzamide (CAS No. 619-57-8, 1.2 g, 8.80 mmol) in DMF (10 mL) were added cesium carbonate (2.60 g, 7.90 mmol) and pent-4-ynyl 4-methylbenzenesulfonate (Intermediate 124, 2.50 g, 11.02 mmol) at room temperature. The reaction mixture was heated at 80° C. for 16 h. Progress of reaction was monitored by TLC. After completion of reaction, the mixture was poured into water (20 mL) and extracted with EtOAc (3×30 mL). The combined organic layer was separated and dried over Na₂SO₄, filtered and concentrated under reduced pressure to get the crude which was purified by combi-flash column chromatography eluted with 20% EtOAc in heptane to afford 4-(pent-4-yn-1-yloxy)benzamide (Intermediate 266, 1.1 g, 62%) as an off-white solid. ¹H NMR (400 MHz, DMSO-d₆) δ ppm 1.90-1.92 (m, 2H), 2.33-2.35 (m, 2H), 2.82 (s, 1H), 4.02-4.16 (m, 2H), 6.97 (d, J=8.13 Hz, 2H), 7.15 (br s, 1H), 7.83 (d, J=8.02 Hz, 2H), 7.84 (br s, 1H). Mass spec: m/z 204.1 [M+H]⁺.

### Step 2

### Intermediate 267: 4-((5-(2-(2,6-dioxo-1-((2-(trimethylsilyl)ethoxy)methyl)piperidin-3-yl)-1-oxoisoindolin-4-yl)pent-4-yn-1-yl)oxy)benzamide

To a solution of 4-pent-4-ynoxybenzamide (Intermediate 266, 1 g, 4.90 mmol) and 3-(4-iodo-1-oxo-isoindolin-2-yl)-1-(2-trimethylsilylethoxymethyl)piperidine-2,6-dione (Intermediate 8, 2.50 g, 4.93 mmol) in DMF (10 mL) were added triethylamine (18 g, 180 mmol), PdCl₂(PPh₃)₂ (0.35 g, 0.49 mmol) and CuI (0.09 g, 0.49 mmol) and the reaction mixture was degassed with argon with 10 min. The reaction mixture was stirred at room temperature for 3 h. Progress of reaction was monitored by TLC. After completion of reaction, the mixture was treated with water (60 mL) and extracted with EtOAc (3×60 mL). The organic layer was separated and dried over Na₂SO₄, filtered and concentrated under reduced pressure to get the crude which was purified by combi-flash column chromatography by eluting with 80% EtOAc in heptane to afford 4-((5-(2-(2,6-dioxo-1-((2-(trimethylsilyl)ethoxy)methyl)piperidin-3-yl)-1-oxoisoindolin-4-yl)pent-4-yn-1-yl)oxy)benzamide (Intermediate 267, 1.50 g, 53%) as an off-white solid. ¹H NMR (400 MHz, DMSO-d₆) δ ppm −0.03 (s, 9H), 0.83-0.86 (m, 2H), 1.97-2.10 (m, 3H), 2.36-2.38 (m, 1H), 2.66-2.68 (m, 2H), 3.44-3.45 (m, 2H), 4.17 (s, 2H), 4.22-4.29 (m, 2H), 4.39-4.48 (m, 2H), 5.05-5.22 (m, 2H), 5.23-5.25 (m, 1H), 6.99-7.01 (m, 2H), 7.16 (br s, 1H), 7.53 (t, J=7.45 Hz, 1H), 7.65 (d, J=7.02 Hz, 1H), 7.72 (d, J=8.3 Hz, 2H), 7.79 (br s, 1H). Mass spec: m/z 574.4 [M+H]⁺.

### Step 3

### Intermediate 268: tert-butyl 6-(4-((5-(2-(2,6-dioxo-1-((2-(trimethylsilyl)ethoxy)methyl)piperidin-3-yl)-1-oxoisoindolin-4-yl)pent-4-yn-1-yl)oxy)benzamido)-2-((R)-1-methylpyrrolidin-2-yl)-1H-pyrrolo[3,2-c]pyridine-1-carboxylate

To a solution of tert-butyl (R)-6-bromo-2-(1-methylpyrrolidin-2-yl)-1H-pyrrolo[3,2-c]pyridine-1-carboxylate (Intermediate 4, 0.80 g, 2.10 mmol) in 1,4-dioxane (15 mL) were added cesium carbonate (2 g, 6.30 mmol) and 4-((5-(2-(2,6-dioxo-1-((2-(trimethylsilyl)ethoxy)methyl)piperidin-3-yl)-1-oxoisoindolin-4-yl)pent-4-yn-1-yl)oxy)benzamide (Intermediate 267, 1 g, 2.01 mmol). The reaction was degassed with argon gas for 20 min followed by Xantphos (0.40 g, 0.60 mmol) and Pd₂(dba)₃ (0.30 g, 0.30 mmol) were added. The reaction mixture was again degassed with argon gas for 10 min. The resulting reaction mixture was heated at 100° C. for 2 h. Progress of reaction was monitored by TLC. After completion of reaction, the mixture was concentrated under reduced pressure to get crude which was purified by combi-flash column chromatography, by eluting with at 80% EtOAc in heptane, to obtain tert-butyl 6-(4-((5-(2-(2,6-dioxo-1-((2-(trimethylsilyl)ethoxy)methyl)piperidin-3-yl)-1-oxoisoindolin-4-yl)pent-4-yn-1-yl)oxy)benzamido)-2-((R)-1-methylpyrrolidin-2-yl)-1H-pyrrolo[3,2-c]pyridine-1-carboxylate (Intermediate 268, 0.52 g, 30%) as a green solid. Mass spec: m/z 875.5 [M+H]⁺.

### Step 4

### Example 56: 4-((5-(2-(2,6-dioxopiperidin-3-yl)-1-oxoisoindolin-4-yl)pent-4-yn-1-yl)oxy)-N-(2-((R)-1-methylpyrrolidin-2-yl)-1H-pyrrolo[3,2-c]pyridin-6-yl)benzamide

To a solution of tert-butyl 6-(4-((5-(2-(2,6-dioxo-1-((2-(trimethylsilyl)ethoxy)methyl)piperidin-3-yl)-1-oxoisoindolin-4-yl)pent-4-yn-1-yl)oxy)benzamido)-2-((R)-1-methylpyrrolidin-2-yl)-1H-pyrrolo[3,2-c]pyridine-1-carboxylate (Intermediate 268, 0.50 g, 0.60 mmol) in acetonitrile (10 mL) was added methane sulfonic acid (0.80 g, 9 mmol) and heated the reaction mixture at 50° C. for 2 h. After 2 h the reaction mixture was cooled to room temperature then added N,N′-dimethylethylenediamine (0.30 g, 3 mmol) and triethylamine (1.0 g, 10.04 mmol) at room temperature. The resulting reaction mixture was stirred for 3 h at room temperature. Progress of reaction was monitored by TLC. After completion of reaction, the reaction mixture was concentrated under reduced pressure to obtain the crude product which was purified by preparative HPLC (Method A) to afford 4-((5-(2-(2,6-dioxopiperidin-3-yl)-1-oxoisoindolin-4-yl)pent-4-yn-1-yl)oxy)-N-(2-((R)-1-methylpyrrolidin-2-yl)-1H-pyrrolo[3,2-c]pyridin-6-yl)benzamide (Example 56, 0.11 g, 30%) as an off-white solid. ¹H NMR (400 MHz, DMSO-d₆) δ ppm 1.81-1.88 (m, 3H), 1.97-2.15 (m, 2H), 2.17 (s, 3H), 2.21-2.46 (m, 3H), 2.58-2.62 (m, 2H), 2.64-2.73 (m, 2H), 2.81-3.00 (m, 1H), 3.08-3.28 (m, 2H), 4.22-4.24 (m, 2H), 4.32-4.44 (m, 2H), 5.10-5.13 (m, 1H), 6.38 (s, 1H), 7.06 (m, 2H), 7.49-7.56 (m, 1H), 7.66 (d, J=7.97 Hz, 1H), 7.71 (d, J=7.50 Hz, 1H), 8.04 (m, 2H), 8.17 (s, 1H), 8.49 (s, 1H), 10.26 (br s, 1H), 11.00 (br s, 1H), 11.34 (br s, 1H). Mass spec: m/z 645.1 [M+H]⁺.

### Example 57 was Synthesised Following Scheme 55

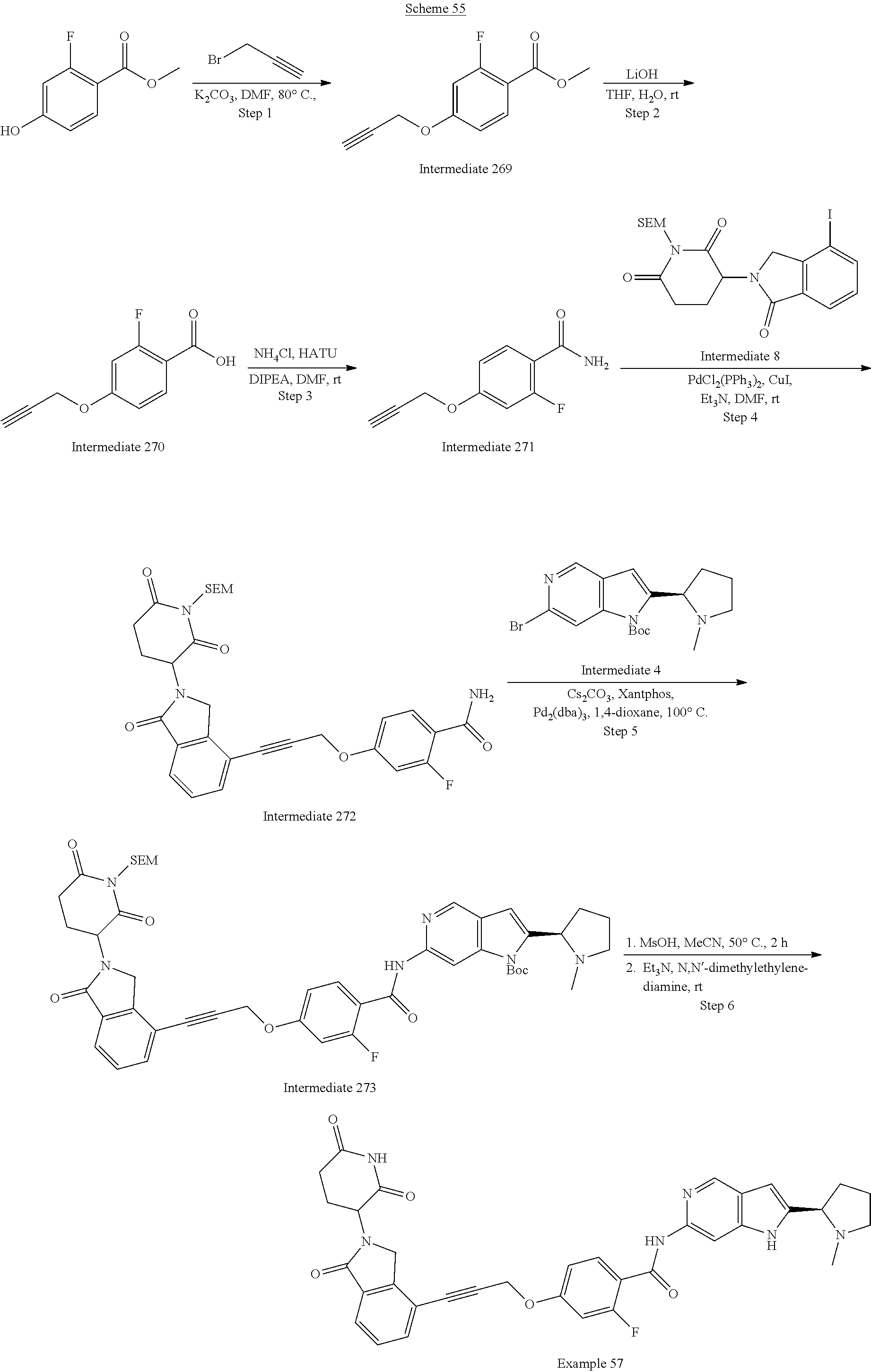

### Step 1

### Intermediate 269: methyl 2-fluoro-4-(prop-2-yn-1-yloxy)benzoate

To a solution of methyl 2-fluoro-4-hydroxybenzoate (CAS No. 197507-22-5, 10.0 g, 58.77 mmol) in DMF (100 mL) was added potassium carbonate (12.3 g, 88.16 mmol) and 3-bromoprop-1-yne (10.48 g, 88.16 mmol). The reaction mixture was heated at 80° C. for 12 h. Progress of the reaction was monitored by TLC. After completion, the reaction mixture was diluted with water (100 mL) and extracted with EtOAc (2×100 mL). The organic layer was separated, dried over anhydrous Na₂SO₄ and concentrated in vacuo. The crude material was purified by combi-flash column chromatography, by eluting with 50% EtOAc in heptane, to afford methyl 2-fluoro-4-(prop-2-yn-1-yloxy)benzoate (Intermediate 269, 8.0 g, 65%) as an off white solid. ¹H NMR (400 MHz, CDCl₃) δ ppm 2.57 (s, 1H), 3.90 (s, 3H), 4.73 (s, 2H), 6.69-6.84 (m, 2H), 7.92-7.93 (m, 1H).

### Step 2

### Intermediate 270: 2-fluoro-4-(prop-2-yn-1-yloxy)benzoic acid

To a solution of methyl 2-fluoro-4-(prop-2-yn-1-yloxy)benzoate (Intermediate 269, 8.0 g, 3 8.42 mmol) in tetrahydrofuran (10 mL) and methanol (10 mL) was added lithium hydroxide (1.87 g, 76.85 mmol) in water (3 mL). Reaction mixture was stirred at room temperature for 12 h. Progress of the reaction was monitored by TLC. The reaction mixture was concentrated in vacuo. The reaction mixture was diluted with water (25 mL). The pH of the aqueous layer was adjusted to 3-4 with 0.5 N HCl and extracted with EtOAc (2×100 mL). The organic layer was dried over Na₂SO₄ and concentrated in vacuo. The crude material was purified by combi-flash column chromatography, by eluting with 30% EtOAc in heptane, to to afford 2-fluoro-4-(prop-2-yn-1-yloxy)benzoic acid (Intermediate 270, 6.0 g, 80%) as an off white solid. ¹H NMR (400 MHz, DMSO-d₆) δ ppm 3.65 (s, 1H), 4.92 (s, 2H), 6.89-6.97 (m, 2H), 7.85-7.86 (m, 1H), 12.94 (br s, 1H). Mass spec: m/z 194.9 [M+H]⁺.

### Step 3

### Intermediate 271: 2-fluoro-4-(prop-2-yn-1-yloxy)benzamide

To a solution of 4-(but-3-yn-1-yl)-2-fluorobenzoic acid (Intermediate 270, 6.0 g, 0.03 mmol) in DMF (80 mL) was added diisopropylethylamine (22.0 mL, 0.12 mmol) and HATU (17.0 g, 0.046 mmol) followed by ammonium chloride (6.6 g, 6.12 mmol). The reaction mixture was stirred at room temperature for 12 h. Progress of the reaction was monitored by TLC. After completion, the reaction mixture was diluted with water (200 mL) and extracted with EtOAc (3×100 mL). The organic layer was separated, dried over anhydrous Na₂SO₄ and concentrated in vacuo. The crude material was purified by combi-flash column chromatography, by eluting with 50% EtOAc in heptane, to afford 2-fluoro-4-(prop-2-yn-1-yloxy)benzamide (Intermediate 271, 5.0 g, 62%) as an off white solid. ¹H NMR (400 MHz, DMSO-d₆) δ ppm 3.63 (s, 1H), 4.89 (s, 2H), 6.86-6.97 (m, 2H), 7.48 (br s, 2H), 7.67-7.68 (m, 1H). Mass spec: m/z 194.0 [M+H]⁺.

### Step 4

### Intermediate 272: 4-((3-(2-(2,6-dioxo-1-((2-(trimethylsilyl) ethoxy)methyl) piperidin-3-yl)-1-oxoisoindolin-4-yl) prop-2-yn-1-yl)oxy)-2-fluorobenzamide

To a solution of 2-fluoro-4-(prop-2-yn-1-yloxy)benzamide (Intermediate 271, 1.0 g, 4.78 mmol) in DMF (10 mL) was added copper(I) iodide (0.09 g, 0.478 mmol) followed by PdCl₂(PPh₃)₂ (0.34 g, 0.47 mmol), 3-(4-iodo-1-oxoisoindolin-2-yl)-1-((2-(trimethylsilyl)ethoxy)methyl)piperidine-2,6-dione (Intermediate 8, 2.39 g, 4.78 mmol) and triethylamine (23.4 mL, 167.00 mmol). The reaction mixture was purged with argon for 20 min. The reaction mixture was stirred at room temperature for 16 h. Progress of the reaction was monitored by TLC. After completion, the reaction mixture was diluted with water (30 mL) and extracted with EtOAc (3×30 mL). The organic layer was separated, dried over anhydrous Na₂SO₄ and concentrated in vacuo. The crude material was purified by combi-flash column chromatography, by eluting with 95% EtOAc in heptane, to afford 4-((3-(2-(2,6-dioxo-1-((2-(trimethylsilyl) ethoxy)methyl) piperidin-3-yl)-1-oxoisoindolin-4-yl) prop-2-yn-1-yl)oxy)-2-fluorobenzamide (Intermediate 272, 1.5 g, 55%) as a white solid. ¹H NMR (400 MHz, DMSO-d₆) δ ppm −0.03 (s, 9H), 0.81-0.86 (m, 2H), 2.03-2.08 (m, 1H), 2.29-2.39 (m, 2H), 2.78-2.82 (m, 1H), 3.03-3.12 (m, 1H), 3.50-3.57 (m, 2H), 4.18-4.39 (m, 2H), 5.19 (s, 2H), 5.19-5.21 (m, 2H), 6.96-7.04 (m, 2H), 7.44 (br s, 1H), 7.49 (br s, 1H), 7.57 (t, J=7.67 Hz, 1H), 7.69-7.73 (m, 2H), 7.79 (d, J=7.46 Hz, 1H).

### Step 5

### Intermediate 273: tert-butyl-6-(4-((3-(2-(2,6-dioxo-1-((2-(trimethylsilyl)ethoxy)methyl)piperidin-3-yl)-1-oxoisoindolin-4-yl)prop-2-yn-1-yl)oxy)-2-fluorobenzamido)-2-((R)-1-methylpyrrolidin-2-yl)-1H-pyrrolo[3,2-c]pyridine-1-carboxylate

To a solution of 4-((3-(2-(2,6-dioxo-1-((2-(trimethylsilyl) ethoxy)methyl) piperidin-3-yl)-1-oxoisoindolin-4-yl) prop-2-yn-1-yl)oxy)-2-fluorobenzamide (Intermediate 272, 0.7 g, 1.23 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.47 g, 1.23 mmol) followed by cesium carbonate (1.21 g, 3.71 mmol). The reaction mixture was purged with argon for 20 min then added Pd₂(dba)₃ (0.25 g, 0.27 mmol) and Xantphos (0.22 g, 0.37 mmol). The reaction mixture was again purged with argon for 10 min and heated at 100° C. for 2 h. Progress of the reaction was monitored by TLC. After completion, the reaction mixture was diluted with water (100 mL), extracted with EtOAc (3×100 mL). The organic layer was separated, dried over anhydrous Na₂SO₄ and concentrated in vacuo. The crude material was purified by combi-flash column chromatography, by eluting with 50% EtOAc in heptane, to afford tert-butyl 6-(4-((3-(2-(2,6-dioxo-1-((2-(trimethylsilyl)ethoxy)methyl)piperidin-3-yl)-1-oxoisoindolin-4-yl)prop-2-yn-1-yl)oxy)-2-fluorobenzamido)-2-((R)-1-methylpyrrolidin-2-yl)-1H-pyrrolo[3,2-c]pyridine-1-carboxylate (Intermediate 273, 0.5 g, 47%) as an off white solid. ¹H NMR (400 MHz, DMSO-d₆) δ ppm −0.07 (s, 9H), 0.74-0.80 (m, 2H), 1.15-1.19 (m, 1H), 1.69 (s, 9H), 1.72-1.79 (m, 2H), 2.03-2.11 (m, 1H), 2.30-2.33 (m, 1H), 2.35 (s, 3H), 2.36-2.43 (m, 2H), 2.72-2.80 (m, 1H), 3.04-3.16 (m, 2H), 3.42-3.48 (m, 2H), 3.90-3.94 (m, 1H), 4.18-4.22 (m, 1H), 4.40-4.44 (m, 1H), 4.96-5.04 (m, 2H), 5.24 (s, 2H), 5.28-5.29 (m, 1H), 6.74 (s, 1H), 7.03 (d, J=8.68, 1H), 7.11-7.12 (m, 1H), 7.52-7.64 (m, 1H), 7.72-7.81 (m, 3H), 8.55 (s, 1H), 8.92 (s, 1H), 10.36 (s, 1H). Mass spec: m/z 865.1 [M]⁺.

### Step 6

### Example 57: 4-((3-(2-(2,6-dioxopiperidin-3-yl)-1-oxoisoindolin-4-yl)prop-2-yn-1-yl)oxy)-2-fluoro-N-(2-((R)-1-methylpyrrolidin-2-yl)-1H-pyrrolo[3,2-c]pyridin-6-yl)benzamide

To a solution of tert-butyl 6-(4-((3-(2-(2,6-dioxo-1-((2-(trimethylsilyl)ethoxy)methyl)piperidin-3-yl)-1-oxoisoindolin-4-yl)prop-2-yn-1-yl)oxy)-2-fluorobenzamido)-2-((R)-1-methylpyrrolidin-2-yl)-1H-pyrrolo[3,2-c]pyridine-1-carboxylate (Intermediate 273, 0.5 g, 0.57 mmol) in acetonitrile (10 mL) was added methane sulfonic acid (0.57 mL, 8.67 mmol) at room temperature and the reaction mixture was heated at 50° C. for 2 h. To the reaction mixture was added triethylamine (0.88 g, 8.67 mmol) followed by N,N-dimethylethylenediamine (0.41 mL, 3.46 mmol) and the reaction mixture was stirred at room temperature for 3 h. Progress of the reaction was monitored by TLC. After completion, the reaction mixture was poured into ice cold water, a soli precipitated and this was filtered, and dried to obtain the crude compound.

The crude material was purified by preparative HPLC (Method A) to afford 4-((3-(2-(2,6-dioxopiperidin-3-yl)-1-oxoisoindolin-4-yl)prop-2-yn-1-yl)oxy)-2-fluoro-N-(2-((R)-1-methylpyrrolidin-2-yl)-1H-pyrrolo[3,2-c]pyridin-6-yl)benzamide (Example 57, 0.05 g, 14%) as an off white solid. ¹H NMR (400 MHz, DMSO-d₆) δ ppm 1.77-1.94 (m, 3H), 2.02-2.05 (m, 1H), 2.12-2.14 (m, 1H), 2.16 (s, 3H), 2.22-2.29 (m, 1H), 2.39-2.43 (m, 1H), 2.59-2.63 (m, 1H), 2.88-2.97 (m, 1H), 3.12-3.16 (m, 1H), 3.28-3.30 (m, 1H), 4.29-4.33 (m, 1H), 4.43-4.48 (m, 1H), 5.16-5.20 (m, 1H), 5.25 (s, 2H), 6.40 (s, 1H), 7.04-7.05 (m, 1H), 7.14-7.15 (m, 1H), 7.57-7.58 (m, 1H), 7.74 (d, J=7.30 Hz, 1H), 7.77-7.82 (m, 2H), 8.20 (s, 1H), 8.47 (s, 1H), 10.03 (br s, 1H), 11.28 (br s, 1H), 11.41 (br s, 1H). Mass spec: m/z 633.0 [M−H]f.

### Example 58 was Synthesised Following Scheme 56

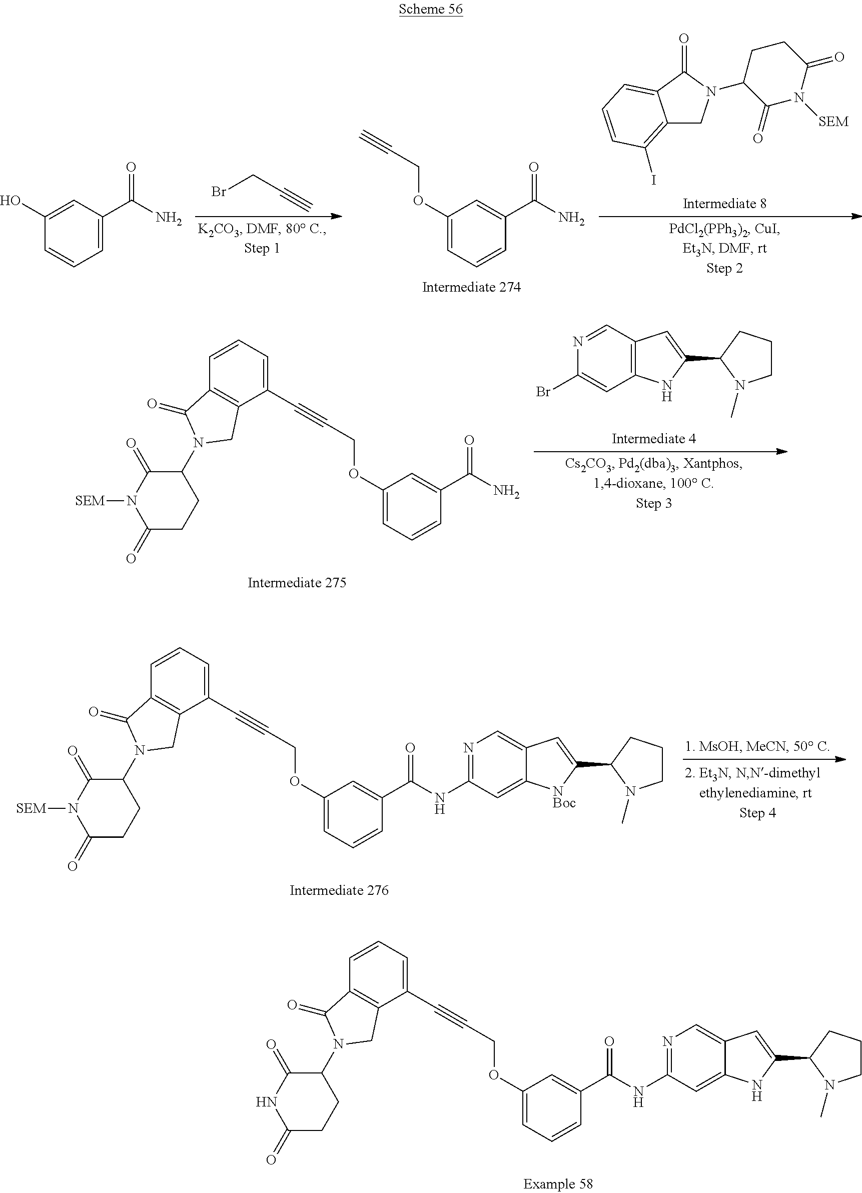

### Step 1

Intermediate 274: 3-(prop-2-yn-1-yloxy)benzamide To a solution of 3-hydroxybenzamide (CAS No: 618-49-5, 5 g, 36.12 mmol) in DMF (10 mL) was added potassium carbonate (7.60 g, 55 mmol) and bromoprop-1-yne (CAS No: 106-96-7, 4.3 g, 36.10 mmol) at room temperature. The resulting reaction mixture was heated at 80° C. for 16 h. The reaction mixture was then treated with water (100 mL) and extracted with EtOAc (2×100 mL). The organic layer was separated and dried over Na₂SO₄, filtered and concentrated under reduced pressure to afford the crude compound which was purified by using combi-flash column chromatography, eluting with 60% EtOAc in heptane, to afford 3-(prop-2-yn-1-yloxy)benzamide (Intermediate 274, 4.80 g, 75%) as an off-white solid. ¹H NMR (400 MHz, DMSO-d₆) δ ppm 7.13 (d, J=8 Hz, 1H), 7.35-7.39 (m, 2H), 7.48 (br s, 1H), 7.49 (d, J=8.10 Hz, 1H), 7.93 (s, 1H). Mass spec: m/z 175.1 [M+H]⁺.

### Step 2

### Intermediate 275: 3-((3-(2-(2,6-dioxo-1-((2-(trimethylsilyl)ethoxy)methyl)piperidin-3-yl)-1-oxoisoindolin-4-yl)prop-2-yn-1-yl)oxy)benzamide

To a solution of 3-(prop-2-yn-1-yloxy)benzamide (Intermediate 274, 1.2 g, 6.90 mmol) and 3-(4-iodo-1-oxoisoindolin-2-yl)-1-((2-(trimethylsilyl)ethoxy)methyl)piperidine-2,6-dione (Intermediate 8, 3.40 g, 6.90 mmol) in DMF (10 mL) was added triethylamine (25 g, 250 mmol) at room temperature followed by the addition of CuI (0.13 g, 0.69 mmol) an PdCl₂(PPh₃)₂ (0.49 g, 0.69 mmol). The reaction mixture was degassed for 5 minutes with argon. The resulting reaction mixture was then stirred at room temperature for 3 h. The reaction was then treated with water (50 mL) and extracted with EtOAc (3×60 mL). The organic layers were separated and dried over Na₂SO₄, filtered and concentrated under reduced pressure to afford the crude compound which was purified by combi-flash column chromatography by eluting with 80% EtOAc in heptane to afford 3-((3-(2-(2,6-dioxo-1-((2-(trimethylsilyl)ethoxy)methyl)piperidin-3-yl)-1-oxoisoindolin-4-yl)prop-2-yn-1-yl)oxy)benzamide (Intermediate 275, 1.30 g, 35%) as a green solid. ¹H NMR (400 MHz, DMSO-d₆) δ ppm −0.14-0.10 (m, 9H), 0.82-0.86 (m, 2H), 2.30-2.35 (m, 1H), 2.80-2.84 (m, 1H), 3.04-3.07 (m, 1H), 3.51-3.56 (m, 2H), 4.02-4.03 (m, 1H), 4.10-4.14 (m, 1H), 4.28-4.33 (m, 1H), 5.05-5.12 (m, 2H), 5.16 (s, 2H), 5.21-5.26 (m, 1H), 7.20 (d, J=8.10 Hz, 1H), 7.32 (s, 1H), 7.37-7.40 (m, 1H), 7.48-7.62 (m, 3H), 7.71 (d, J=8.12 Hz, 1H), 7.78 (d, J=7.46 Hz, 1H), 7.97 (s, 1H). Mass spec: m/z 546 [M+H]⁺.

### Step 3

### Intermediate 276: tert-butyl 6-(3-((3-(2-(2,6-dioxo-1-((2-(trimethylsilyl)ethoxy)methyl)piperidin-3-yl)-1-oxoisoindolin-4-yl)prop-2-yn-1-yl)oxy)benzamido)-2-((R)-1-methylpyrrolidin-2-yl)-1H-pyrrolo[3,2-c]pyridine-1-carboxylate

To a solution of tert-butyl (R)-6-bromo-2-(1-methylpyrrolidin-2-yl)-1H-pyrrolo[3,2-c]pyridine-1-carboxylate (Intermediate 4, 1 g, 2.6 mmol) and 3-((3-(2-(2,6-dioxo-1-((2-(trimethylsilyl) ethoxy)methyl)piperidin-3-yl)-1-oxoisoindolin-4-yl) prop-2-yn-1-yl)oxy)benzamide (Intermediate 275, 0.80 g, 1 mmol) in 1,4-dioxane (20 mL) was added caesium carbonate (2 g, 6 mmol) and the resulting reaction mixture was degassed with argon for 20 min followed. Xanthphos (0.30 g, 0.60 mmol) and Pd₂(dba)₃ (0.60 g, 0.60 mmol) were added and the reaction mixture was again degassed with argon for 10 min then heated at 100° C. for 2 h. The reaction mixture was concentrated under reduced pressure to get the crude material which was purified by combi-flash column chromatography by eluting with 80% EtOAc and heptane to afford tert-butyl 6-(3-((3-(2-(2,6-dioxo-1-((2-(trimethylsilyl)ethoxy)methyl)piperidin-3-yl)-1-oxoisoindolin-4-yl)prop-2-yn-1-yl)oxy)benzamido)-2-((R)-1-methylpyrrolidin-2-yl)-1H-pyrrolo[3,2-c]pyridine-1-carboxylate (Intermediate 276, 0.85 g, 50%) as a yellow solid. ¹H NMR (400 MHz, DMSO-d₆) δ ppm −0.14-0.01 (m, 9H), 0.75-0.82 (m, 2H), 1.61-1.93 (m, 11H), 1.95-1.98 (m, 1H), 2.17-2.30 (m, 1H), 2.30-2.49 (m, 5H), 2.58-2.64 (m, 1H), 2.95-2.96 (m, 1H), 3.13-3.30 (m, 2H), 3.40-3.46 (m, 2H), 3.90-3.93 (m, 1H), 4.01-4.06 (m, 1H), 4.26-4.30 (m, 1H), 4.83-4.94 (m, 2H), 5.12-5.16 (m, 1H), 5.24 (s, 2H), 6.75 (s, 1H), 7.24-7.26 (m, 1H), 7.38-7.43 (m, 1H), 7.36-7.43 (m, 1H), 7.43-7.58 (m, 1H), 7.58-7.78 (m, 2H), 7.84 (s, 1H), 8.59 (s, 1H), 8.92 (s, 1H), 10.78 (s, 1H). Mass spec: m/z 847.1 [M+H]⁺.

### Step 4

### Example 58: 3-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)benzamide

To a solution of tert-butyl 6-(3-((3-(2-(2,6-dioxo-1-((2-(trimethylsilyl)ethoxy)methyl)piperidin-3-yl)-1-oxoisoindolin-4-yl)prop-2-yn-1-yl)oxy)benzamido)-2-((R)-1-methylpyrrolidin-2-yl)-1H-pyrrolo[3,2-c]pyridine-1-carboxylate (Intermediate 276, 0.60 g, 0.70 mmol) in acetonitrile (15 mL) was added methanesulfonic acid (0.69 g, 7.08 mmol) at room temperature. The reaction mixture was then heated to 50° C. for 2 h before cooling to room temperature. N,N′-dimethylethylenediamine (0.34 g, 3.54 mmol) and triethylamine (1.44 g, 14.20 mmol) were added and the resulting reaction mixture was stirred at room temperature for 3 h. The reaction mixture was concentrated under reduced pressure to afford the crude compound which was purified by preparative HPLC (Method A) to afford 3-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)benzamide (Example 58, 0.51 g, 19%) as an off-white solid. ¹H NMR (400 MHz, DMSO-d₆) δ ppm 1.78-1.95 (m, 4H), 2.12-2.14 (m, 2H), 2.15 (s, 3H), 2.23-2.30 (m, 2H), 2.78-2.86 (m, 1H), 3.12-3.16 (m, 1H), 4.14-4.19 (m, 1H), 4.32-4.36 (m, 1H), 5.03-5.08 (m, 1H), 5.24 (s, 2H), 6.39 (s, 1H), 7.24-7.27 (m, 1H), 7.44 (t, J=8.10 Hz, 1H), 7.55 (t, J=8.10, 1H), 7.67-7.79 (m, 4H), 8.17 (s, 1H), 8.50 (s, 1H), 10.40 (s, 1H), 11.05 (s, 1H), 11.40 (s, 1H). Mass spec: m/z 617 [M+H]⁺.

### Example 59 was Synthesised Following Scheme 57

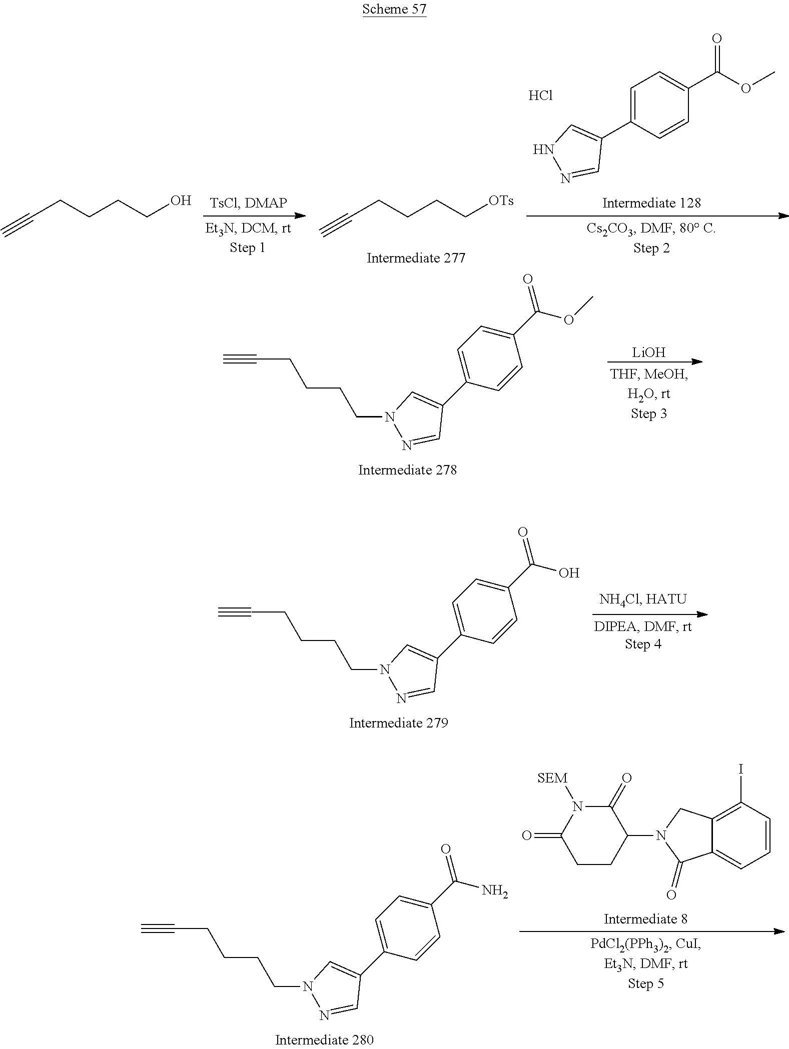

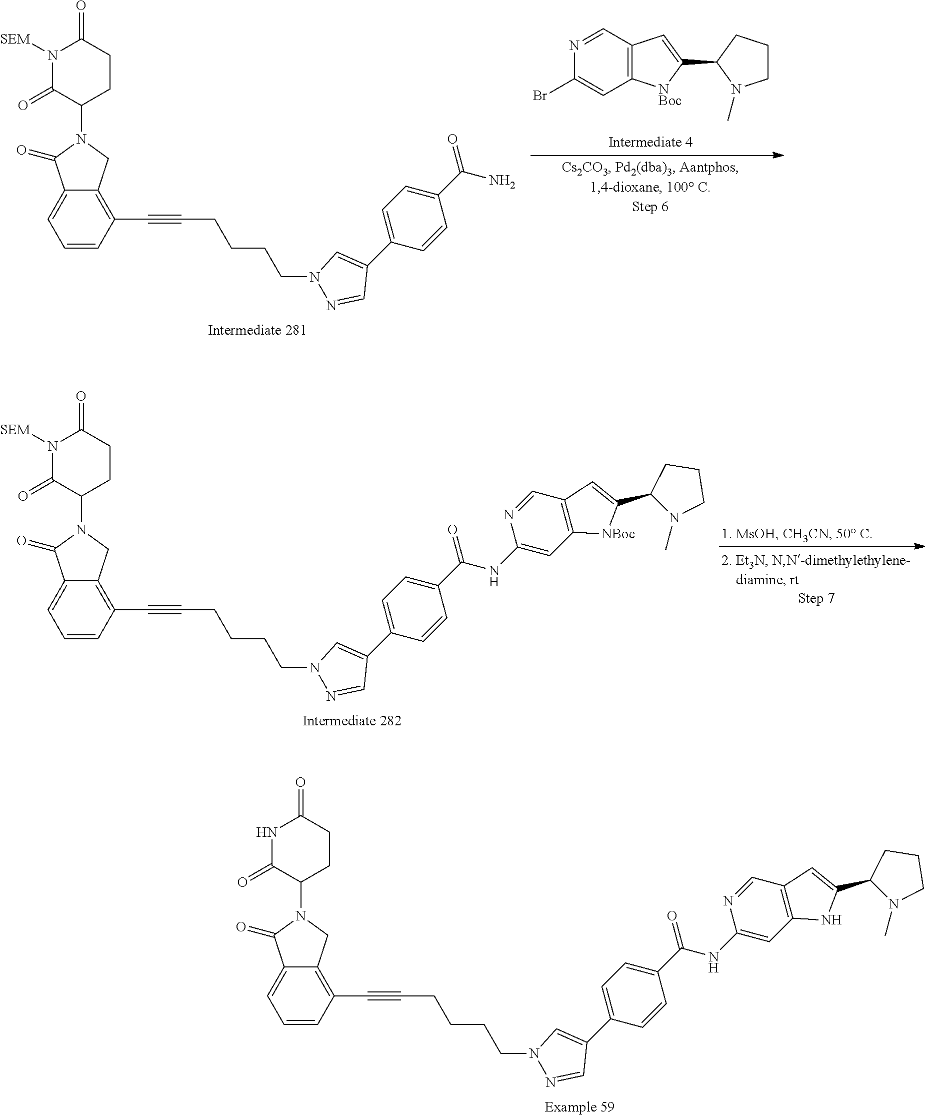 [SMILES: C[Si](C)(C)CCOCN1C(=O)CCC(N2Cc3c(C#CCCCCn4cc(-c5ccc(C(N)=O)cc5)cn4)cccc3C2=O)C1=O ; CN1CCC[C@@H]1c1cc2cnc(Br)cc2n1C(=O)OC(C)(C)C ; CN1CCC[C@@H]1c1cc2cnc(NC(=O)c3ccc(-c4cnn(CCCCC#Cc5cccc6c5CN(C5CCC(=O)N(COCC[Si](C)(C)C)C5=O)C6=O)c4)cc3)cc2n1C(=O)OC(C)(C)C ; CN1CCC[C@@H]1c1cc2cnc(NC(=O)c3ccc(-c4cnn(CCCCC#Cc5cccc6c5CN(C5CCC(=O)NC5=O)C6=O)c4)cc3)cc2[nH]1]

### Step 1

### Intermediate 277: hex-5-yn-1-yl 4-methylbenzenesulfonate

To a cooled (0° C.) solution of hex-5-yn-1-ol (CAS No: 928-90-5, 5.00 g, 51 mmol) in dichloromethane (50 mL) was added p-toluenesulfonyl chloride (15.0 g, 76.0 mmol) followed by triethylamine (21 mL, 150 mmol) and 4-dimethylaminopyridine (0.66 g, 5.1 mmol). The reaction mixture was stirred at room temperature for 16 h then diluted with water (25 mL) and extracted with dichloromethane (3×25 mL). The combined organic layers were separated, dried over anhydrous Na₂SO₄, and concentrated in vacuo. The crude material was purified by combi-flash chromatography, by eluting with 55% ethyl acetate in heptane, to afford hex-5-yn-1-yl 4-methylbenzenesulfonate (Intermediate 277, 7.00 g, 54%) as a yellow oil. ¹H NMR (400 MHz, DMSO-d₆) δ ppm 1.39-1.44 (m, 2H), 1.61-1.66 (m, 2H), 2.08-2.13 (m, 2H), 2.42 (s, 3H), 2.74 (s, 1H), 4.04 (t, J=6.40 Hz, 2H), 7.48 (d, J=8.40 Hz, 2H), 7.78 (d, J=8.40 Hz, 2H).

### Step 2

### Intermediate 278: methyl-4-(1-(hex-5-yn-1-yl)-1H-pyrazol-4-yl)benzoate

To a cooled (0° C.) solution of methyl 4-(1H-pyrazol-4-yl)benzoate hydrochloride (Intermediate 128, 4.00 g, 16.76 mmol) in dimethylformamide (50 mL) was added hex-5-yn-1-yl 4-methylbenzenesulfonate (Intermediate 277, 6.34 g, 25.14 mmol) followed by cesium carbonate (16.4 g, 50.28 mmol) at 0° C. The reaction mixture was then heated at 80° C. for 16 h. The reaction mixture was then concentrated in vacuo and the obtained residue was poured into water (100 mL). The resultant precipitate was collected by filtration and dried in vacuo to afford methyl-4-(1-(hex-5-yn-1-yl)-1H-pyrazol-4-yl)benzoate (Intermediate 278, 2.70 g, 57%) as an off white solid, which was used for the next step without further purification. ¹H NMR (400 MHz, DMSO-d₆) δ ppm 1.38-1.46 (m, 2H), 1.85-1.93 (m, 2H), 2.17-2.21 (m, 2H), 2.77 (s, 1H), 3.84 (s, 3H), 4.15 (t, J=6.80 Hz, 2H), 7.72 (d, J=8.40 Hz, 2H), 7.93 (d, J=8.40 Hz, 2H), 7.99 (s, 1H), 8.33 (s, 1H). Mass spec m/z 282.9 [M+H]⁺.

### Step 3

### Intermediate 279: 4-(1-(hex-5-yn-1-yl)-1H-pyrazol-4-yl)benzoic acid

To a solution of methyl-4-(1-(hex-5-yn-1-yl)-1H-pyrazol-4-yl)benzoate (Intermediate 278, 2.70 g, 9.56 mmol) in tetrahydrofuran (25 mL), methanol (15 mL) and water (15 mL) was added lithium hydroxide (0.70 g, 28.7 mmol) and the reaction mixture was stirred at room temperature for 16 h then concentrated in vacuo. The obtained residue was diluted with water (10 mL) and acidified to pH~2 with aqueous citric acid (20 mL). The resultant precipitate was collected by filtration and dried in vacuo to afford 4-(1-(hex-5-yn-1-yl)-1H-pyrazol-4-yl)benzoic acid (Intermediate 279, 2.30 g) as a white solid, which was used for the next step without further purification. ¹H NMR (400 MHz, DMSO-d₆) δ ppm 1.35-1.43 (m, 2H), 1.82-1.89 (m, 2H), 2.14-2.18 (m, 2H), 2.73 (s, 1H), 4.12 (t, J=6.80 Hz, 2H), 7.66 (d, J=8.40 Hz, 2H), 7.88 (d, J=8.40 Hz, 2H), 7.95 (s, 1H), 8.28 (s, 1H), 12.76 (br s, 1H). Mass spec m/z 268.9 [M+H]⁺.

### Step 4

### Intermediate 280: 4-(1-(hex-5-yn-1-yl)-1H-pyrazol-4-yl)benzamide

To a solution of 4-(1-(hex-5-yn-1-yl)-1H-pyrazol-4-yl)benzoic acid (Intermediate 279, 2.30 g, 8.6 mmol) in dimethylformamide (25 mL) was added N,N-diisopropylethylamine (7.5 mL, 43 mmol) followed by HATU (4.0 g, 10 mmol) and the reaction mixture was stirred at room temperature for 10 min. Ammonium chloride (2.3 g, 43 mmol) was added and the reaction mixture was stirred at room temperature for 16 h then concentrated in vacuo. The obtained residue was poured into water (100 mL), the resultant precipitate was collected by filtration and dried in vacuo to afford 4-(1-(hex-5-yn-1-yl)-1H-pyrazol-4-yl)benzamide (Intermediate 280, 1.50 g, 65%) as an off-white solid which was used for the next step without further purification. ¹H NMR (400 MHz, DMSO-d₆) δ ppm 1.39-1.46 (m, 2H), 1.85-1.92 (m, 2H), 2.17-2.22 (m, 2H), 2.77 (s, 1H), 4.14 (t, J=6.80 Hz, 2H), 7.27 (br s, 1H), 7.64 (d, J=8.40 Hz, 2H), 7.86 (d, J=8.40 Hz, 2H), 7.91 (br s, 1H), 7.96 (s, 1H), 8.28 (s, 1H). Mass spec m/z 267.9 [M+H]⁺.

### Step 5

### Intermediate 281: 4-(1-(6-(2-(2,6-dioxo-1-((2-(trimethylsilyl)ethoxy)methyl)piperidin-3-yl)-1-oxoisoindolin-4-yl) hex-5-yn-1-yl)-1H-pyrazol-4-yl)benzamide

To a solution of 4-(1-(hex-5-yn-1-yl)-1H-pyrazol-4-yl)benzamide (Intermediate 280, 1.00 g, 3.7 mmol) in dimethylformamide (5 mL) was added 3-(4-iodo-1-oxoisoindolin-2-yl)-1-((2-(trimethylsilyl)ethoxy)methyl)piperidine-2,6-dione (Intermediate 8, 1.9 g, 3.7 mmol), followed by triethylamine (18 mL, 130 mmol). The reaction mixture was purged with argon for 15 min, then PdCl₂(PPh₃)₂ (0.27 g, 0.37 mmol) and copper (I) iodide (0.073 g, 0.37 mmol) were added. The reaction mixture was further purged with argon for 10 min and stirred at room temperature for 2 h. The reaction mixture was diluted with water (100 mL) and extracted with ethyl acetate (3×200 mL). The combined organic layers were separated, dried over anhydrous Na₂SO₄, and concentrated in vacuo. The crude material was purified by combi-flash chromatography, by eluting with 100% ethyl acetate in heptane, to afford 4-(1-(6-(2-(2,6-dioxo-1-((2-(trimethylsilyl)ethoxy)methyl) piperidin-3-yl)-1-oxoisoindolin-4-yl) hex-5-yn-1-yl)-1H-pyrazol-4-yl)benzamide (Intermediate 281, 1.50 g, 63%) as a yellow solid. ¹H NMR (400 MHz, DMSO-d₆) δ ppm −0.07 (s, 9H), 0.76-0.83 (m, 2H), 1.49-1.58 (m, 2H), 1.90-2.05 (m, 3H), 2.36-2.39 (m, 1H), 2.70-2.76 (m, 2H), 2.84-2.86 (m, 1H), 3.98-3.05 (m, 1H), 3.46-3.50 (m, 2H), 4.11-4.26 (m, 3H), 4.42-4.48 (m, 1H), 4.98-5.05 (m, 2H), 5.20-5.25 (m, 1H), 7.25 (br s, 1H), 7.47-7.70 (m, 5H), 7.80-7.84 (m, 2H), 7.88 (br s, 1H), 7.91 (s, 1H), 8.27 (s, 1H). Mass spec m/z 637.9 [M−H]⁻.

### Step 5

### Intermediate 282: tert-butyl 6-(4-(1-(6-(2-(2,6-dioxo-1-((2-(trimethylsilyl)ethoxy)methyl)piperidin-3-yl)-1-oxoisoindolin-4-yl)hex-5-yn-1-yl)-1H-pyrazol-4-yl)benzamido)-2-((R)-1-methylpyrrolidin-2-yl)-1H-pyrrolo[3,2-c]pyridine-1-carboxylate

To a solution of 4-(1-(6-(2-(2,6-dioxo-1-((2-(trimethylsilyl)ethoxy)methyl)piperidin-3-yl)-1-oxoisoindolin-4-yl) hex-5-yn-1-yl)-1H-pyrazol-4-yl)benzamide (Intermediate 281, 0.8 g, 1 mmol) in 1,4-dioxane (15 mL) was added tert-butyl-(R)-6-bromo-2-(1-methylpyrrolidin-2-yl)-1H-pyrrolo[3,2-c]pyridine-1-carboxylate (Intermediate 4, 0.6 g, 2 mmol) followed by cesium carbonate (2 g, 5 mmol). The reaction mixture was purged with argon for 15 min, then Pd₂(dba)₃ (0.2 g, 0.2 mmol) followed by Xantphos (0.3 g, 0.5 mmol) were added. The reaction mixture was purged with argon for 10 min and then heated at 100° C. for 2 h. The reaction mixture was filtered through a celite bed and the filter pad was washed with ethyl acetate (50 mL). The filtrate was concentrated in vacuo and the crude material purified by combi-flash chromatography, by eluting with 100% ethyl acetate in heptane, to afford tert-butyl 6-(4-(1-(6-(2-(2,6-dioxo-1-((2-(trimethylsilyl)ethoxy)methyl)piperidin-3-yl)-1-oxoisoindolin-4-yl)hex-5-yn-1-yl)-1H-pyrazol-4-yl)benzamido)-2-((R)-1-methylpyrrolidin-2-yl)-1H-pyrrolo[3,2-c]pyridine-1-carboxylate (Intermediate 282, 0.40 g, 30%) as a yellow solid. ¹H NMR (400 MHz, DMSO-d₆) δ ppm −0.04 (s, 9H), 0.79-0.82 (m, 2H), 1.55-1.62 (m, 5H), 1.70 (s, 9H), 1.73-1.75 (m, 2H), 1.98-2.04 (m, 4H), 2.36 (s, 3H), 2.42-2.45 (m, 2H), 2.74-2.78 (m, 1H), 3.01-3.14 (m, 2H), 3.46-3.56 (m, 2H), 4.20 (t, J=6.80 Hz, 2H), 4.26-4.30 (m, 2H), 4.46-4.50 (m, 1H), 5.00-5.08 (m, 2H), 5.23-528 (m, 1H), 6.76 (s, 1H), 7.53 (t, J=8.0 Hz, 1H), 7.64-7.73 (m, 4H), 7.99 (s, 1H), 8.05 (d, J=8.0 Hz, 2H), 8.35 (s, 1H), 8.60 (s, 1H), 8.94 (s, 1H), 10.61 (br s, 1H). Mass spec m/z 939.0 [M+H]⁺.

### Step 7

### Example 59: 4-(1-(6-(2-(2,6-dioxopiperidin-3-yl)-1-oxoisoindolin-4-yl)hex-5-yn-1-yl)-1H-pyrazol-4-yl)-N-(2-((R)-1-methylpyrrolidin-2-yl)-1H-pyrrolo[3,2-c]pyridin-6-yl)benzamide

To a solution of tert-butyl 6-(4-(1-(6-(2-(2,6-dioxo-1-((2-(trimethylsilyl)ethoxy)methyl)piperidin-3-yl)-1-oxoisoindolin-4-yl)hex-5-yn-1-yl)-1H-pyrazol-4-yl)benzamido)-2-((R)-1-methylpyrrolidin-2-yl)-1H-pyrrolo[3,2-c]pyridine-1-carboxylate (Intermediate 282, 0.40 g, 0.43 mmol) in acetonitrile (8 mL) was added methanesulfonic acid (0.42 mL, 6.4 mmol) and the reaction mixture was heated at 50° C. for 2 h. The reaction mixture was cooled to room temperature, then N,N′-dimethylethylenediamine (0.31 mL, 2.6 mmol) followed by triethylamine (1.2 mL, 8.5 mmol) were added and the mixture stirred at room temperature for 3 h. The reaction mixture was concentrated in vacuo and the resultant residue was diluted with water (50 mL). The resultant precipitate was collected by filtration and dried in vacuo. The crude solid was purified by preparative HPLC (Method A) to afford 4-(1-(6-(2-(2,6-dioxopiperidin-3-yl)-1-oxoisoindolin-4-yl)hex-5-yn-1-yl)-1H-pyrazol-4-yl)-N-(2-((R)-1-methylpyrrolidin-2-yl)-1H-pyrrolo[3,2-c]pyridin-6-yl)benzamide (Example 59, 0.19 g, 63%) as an off white solid. ¹H NMR (400 MHz, DMSO-d₆) δ ppm 1.54-1.61 (m, 2H), 1.78-1.94 (m, 3H), 1.99-2.06 (m, 3H), 2.13-2.15 (m, 1H), 2.17 (s, 3H), 2.23-2.30 (m, 1H), 2.39-2.46 (m, 2H), 2.52-2.59 (m, 3H), 2.86-2.95 (m, 1H), 3.12-3.19 (m, 1H), 4.21 (t, J=6.82 Hz, 2H), 4.30-4.34 (m, 1H), 4.44-4.49 (m, 1H), 5.11-5.16 (m, 1H), 6.39 (s, 1H), 7.50-7.54 (m, 1H), 7.63-7.65 (m, 1H), 7.68-7.73 (m, 3H), 7.99 (s, 1H), 8.00-8.05 (m, 2H), 8.20 (s, 1H), 8.35 (s, 1H), 8.50 (s, 1H), 10.38 (s, 1H), 11.00 (s, 1H), 11.36 (s, 1H). Mass spec m/z 709.3 [M+H]⁺.

### Example 60 was Synthesised Following Scheme 58

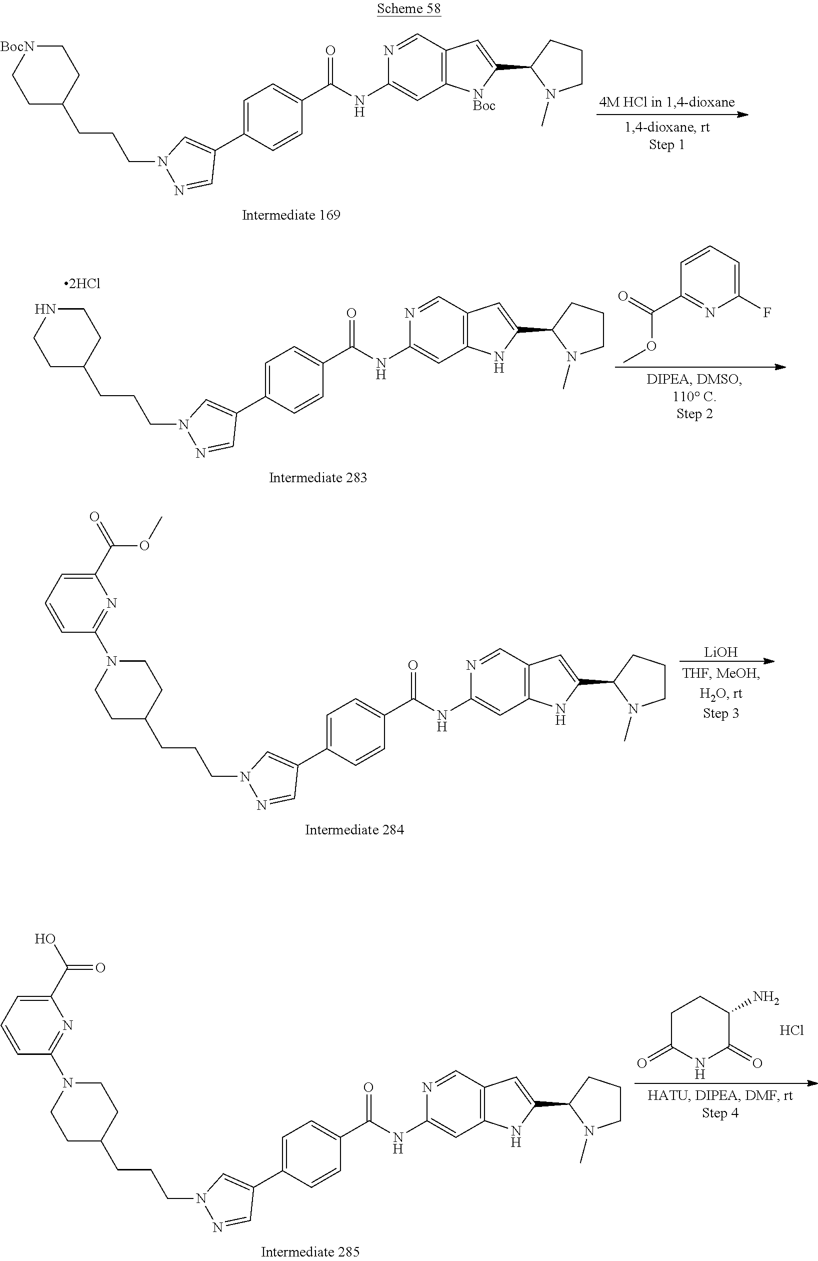

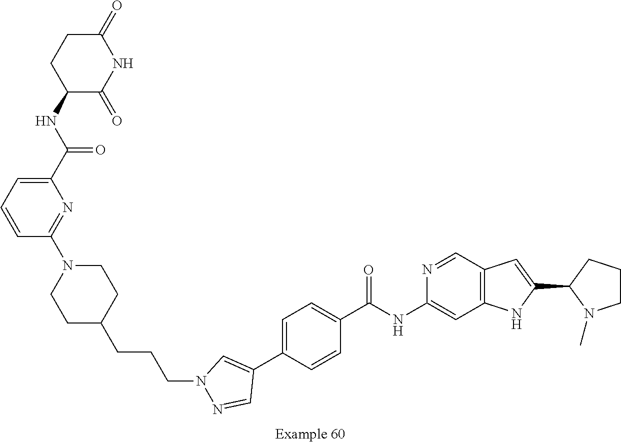 [SMILES: CN1CCC[C@@H]1c1cc2cnc(NC(=O)c3ccc(-c4cnn(CCCC5CCN(c6cccc(C(=O)N[C@H]7CCC(=O)NC7=O)n6)CC5)c4)cc3)cc2[nH]1]

### Step 1

### Intermediate 283: (R)—N-(2-(1-methylpyrrolidin-2-yl)-1H-pyrrolo[3,2-c]pyridin-6-yl)-4-(1-(3-(piperidin-4-yl) propyl)-1H-pyrazol-4-yl)benzamide dihydrochloride

To a cooled (0° C.) solution of tert-butyl-(R)-6-(4-(1-(3-(1-(tert-butoxycarbonyl)piperidin-4-yl)propyl)-1H-pyrazol-4-yl)benzamido)-2-(1-methylpyrrolidin-2-yl)-1H-pyrrolo[3,2-c]pyridine-1-carboxylate (Intermediate 169, 1.40 g, 2.0 mmol) in 1,4-dioxane (10 mL) was added 4M hydrochloric acid in 1,4-dioxane (15 mL) and the reaction mixture was stirred at room temperature for 16 h. The reaction mixture was concentrated in vacuo to afford (R)—N-(2-(1-methylpyrrolidin-2-yl)-1H-pyrrolo[3,2-c]pyridin-6-yl)-4-(1-(3-(piperidin-4-yl) propyl)-1H-pyrazol-4-yl)benzamide dihydrochloride (Intermediate 283, 1.20 g) as an off-white solid which was used for the next step without further purification. ¹H NMR (400 MHz, DMSO-d₆) δ ppm 1.12-1.34 (m, 4H), 1.45-1.61 (m, 1H), 1.70-1.89 (m, 4H), 2.12-2.20 (m, 2H), 2.32-2.40 (m, 2H), 2.49 (s, 3H), 2.78-2.81 (m, 2H), 3.20-3.24 (m, 2H), 3.69-3.71 (m, 1H), 3.72-3.75 (m, 1H), 4.12 (t, J=6.40 Hz, 2H), 4.70-4.80 (m, 1H), 7.10 (s, 1H), 7.82 (d, J=8.0 Hz, 2H), 8.06 (s, 1H), 8.21 (d, J=8.0 Hz, 2H), 8.27 (s, 1H), 8.40 (s, 1H), 8.60 (br s, 1H), 8.83 (br s, 1H), 9.10 (s, 1H), 11.42 (br s, 1H), 12.0 (br s, 1H), 13.29 (br s, 1H). Mass spec m/z 512.1 [M+H]⁺.

### Step 2

### Intermediate 284: methyl-(R)-6-(4-(3-(4-(4-((2-(1-methylpyrrolidin-2-yl)-1H-pyrrolo[3,2-c]pyridin-6-yl)carbamoyl)phenyl)-1H-pyrazol-1-yl)propyl)piperidin-1-yl)picolinate

To a solution of (R)—N-(2-(1-methylpyrrolidin-2-yl)-1H-pyrrolo[3,2-c]pyridin-6-yl)-4-(1-(3-(piperidin-4-yl) propyl)-1H-pyrazol-4-yl)benzamide dihydrochloride (Intermediate 283, 0.80 g, 1.56 mmol) in dimethylsulfoxide (10 mL) was added methyl-6-fluoropicolinate (CAS No: 455-71-0, 0.31 g, 2.03 mmol) followed by N,N-diisopropylethylamine (1.37 mL, 7.82 mmol). The reaction mixture was heated at 110° C. for 16 h then poured into water (100 mL). The resultant precipitate was collected by filtration and dried in vacuo to afford methyl-(R)-6-(4-(3-(4-(4-((2-(1-methylpyrrolidin-2-yl)-1H-pyrrolo[3,2-c]pyridin-6-yl)carbamoyl)phenyl)-1H-pyrazol-1-yl)propyl)piperidin-1-yl)picolinate (Intermediate 284, 0.35 g, 35%) as a brown solid, which was used for the next step without further purification. ¹H NMR (400 MHz, DMSO-d₆) δ ppm 1.01-1.15 (m, 3H), 1.18-1.24 (m, 2H), 1.47-1.58 (m, 1H), 1.69-1.97 (m, 6H), 2.13-2.27 (m, 3H), 2.54 (s, 3H), 2.76-2.80 (m, 2H), 3.12-3.16 (m, 1H), 3.81 (s, 3H), 4.13 (t, J=6.80 Hz, 2H), 4.32-4.36 (m, 2H), 6.40 (s, 1H), 7.05 (d, J=8.80 Hz, 1H), 7.24 (d, J=7.60 Hz, 1H), 7.63 (t, J=8.0 Hz, 1H), 7.70 (d, J=8.0 Hz, 2H), 8.0 (s, 1H), 8.06 (d, J=7.60 Hz, 2H), 8.20 (s, 1H), 8.34 (s, 1H), 8.51 (s, 1H), 10.39 (br s, 1H), 11.37 (br s, 1H). Mass spec m/z 647.3 [M+H]⁺.

### Step 3

### Intermediate 285: (R)-6-(4-(3-(4-(4-((2-(1-methylpyrrolidin-2-yl)-1H-pyrrolo[3,2-c]pyridin-6-yl)carbamoyl)phenyl)-1H-pyrazol-1-yl)propyl)piperidin-1-yl)picolinic acid

To a cooled (0° C.) solution of methyl-(R)-6-(4-(3-(4-(4-((2-(1-methylpyrrolidin-2-yl)-1H-pyrrolo[3,2-c]pyridin-6-yl)carbamoyl)phenyl)-1H-pyrazol-1-yl)propyl)piperidin-1-yl)picolinate (Intermediate 284, 0.35 g, 0.54 mmol) in tetrahydrofuran (5 mL), methanol (2 mL) and water (3 mL) was added lithium hydroxide (0.039 g, 1.62 mmol) and the reaction mixture was stirred at room temperature for 16 h. The reaction mixture was concentrated in vacuo and the obtained residue was diluted in water (10 mL) and acidified to ~pH4 by the addition of 1N aqueous HCl solution. The resultant precipitate was collected by filtration and dried in vacuo to afford (R)-6-(4-(3-(4-(4-((2-(1-methylpyrrolidin-2-yl)-1H-pyrrolo[3,2-c]pyridin-6-yl)carbamoyl)phenyl)-1H-pyrazol-1-yl)propyl)piperidin-1-yl)picolinic acid (Intermediate 285, 0.20 g, 58%) as an off brown solid, which was used for the next step without further purification. ¹H NMR (400 MHz, DMSO-d₆) δ ppm 1.01-1.13 (m, 2H), 1.18-1.30 (m, 2H), 1.47-1.58 (m, 1H), 1.66-2.01 (m, 6H), 2.08-2.25 (m, 3H), 2.30-2.42 (m, 2H), 2.54 (s, 3H), 3.12-3.20 (m, 1H), 4.13 (t, J=6.40 Hz, 2H), 4.35-4.45 (m, 2H), 4.68-4.63 (m, 1H), 6.15 (s, 1H), 7.02 (d, J=8.0 Hz, 1H), 7.23 (d, J=6.80 Hz, 1H), 7.62 (t, J=8.0 Hz, 1H), 7.74 (d, J=8.0 Hz, 2H), 8.01 (s, 1H), 8.14 (d, J=8.0 Hz, 2H), 8.34 (s, 1H), 8.38 (s, 1H), 8.81 (s, 1H), 11.07 (br s, 1H), 11.56 (br s, 1H), 12.63 (br s, 1H). Mass spec m/z 633.1 [M+H]⁺.

### Step 4

### Example 60: N—((S)-2,6-dioxopiperidin-3-yl)-6-(4-(3-(4-(4-((2-((R)-1-methylpyrrolidin-2-yl)-1H-pyrrolo[3,2-c]pyridin-6-yl)carbamoyl)phenyl)-1H-pyrazol-1-yl)propyl)piperidin-1-yl)picolinamide

To a cooled (0° C.) solution of (R)-6-(4-(3-(4-(4-((2-(1-methylpyrrolidin-2-yl)-1H-pyrrolo[3,2-c]pyridin-6-yl)carbamoyl)phenyl)-1H-pyrazol-1-yl)propyl)piperidin-1-yl)picolinic acid (Intermediate 285, 0.20 g, 0.31 mmol) in dimethylformamide (10 mL) was added HATU (0.18 g, 0.47 mmol) and N,N-diisopropylethylamine (0.22 mL, 1.26 mmol). The reaction mixture was stirred for 10 min, then (S)-3-aminopiperidine-2,6-dione hydrochloride (CAS No: 25181-50-4, 0.062 g, 0.38 mmol) was added at 0° C. and the reaction mixture was stirred at room temperature for 16 h. The reaction mixture was poured into water (100 mL), the resultant precipitate was collected by filtration and dried in vacuo. The crude solid was purified by preparative HPLC (Method A) to afford N—((S)-2,6-dioxopiperidin-3-yl)-6-(4-(3-(4-(4-((2-((R)-1-methylpyrrolidin-2-yl)-1H-pyrrolo[3,2-c]pyridin-6-yl)carbamoyl)phenyl)-1H-pyrazol-1-yl)propyl)piperidin-1-yl)picolinamide (Example 60, 0.02 g, 9%) as an off white solid. ¹H NMR (400 MHz, DMSO-d₆) δ ppm 1.08-1.18 (m, 2H), 1.20-1.30 (m, 2H), 1.48-1.58 (m, 1H), 1.71-1.82 (m, 3H), 1.83-1.95 (m, 4H), 1.97-2.05 (m, 2H), 2.17 (s, 3H), 2.20-2.30 (m, 3H), 2.52-2.56 (m, 2H), 2.73-2.82 (m, 2H), 3.11-3.19 (m, 1H), 4.14 (t, J=6.0 Hz, 2H), 4.38-4.42 (m, 2H), 4.67-4.76 (m, 1H), 6.39 (s, 1H), 7.02 (d, J=8.80 Hz, 1H), 7.26 (d, J=7.60 Hz, 1H), 7.64 (t, J=7.60 Hz, 1H), 7.71 (d, J=8.80 Hz, 2H), 8.0 (s, 1H), 8.06 (d, J=7.60 Hz, 2H), 8.20 (s, 1H), 8.34 (s, 1H), 8.50 (s, 1H), 8.73 (d, J=8.80 Hz, 1H), 10.38 (br s, 1H), 10.87 (br s, 1H), 11.36 (br s, 1H). Mass spec m/z 743.2 [M+H]⁺.

### Example 61 was Synthesised Following Scheme 59

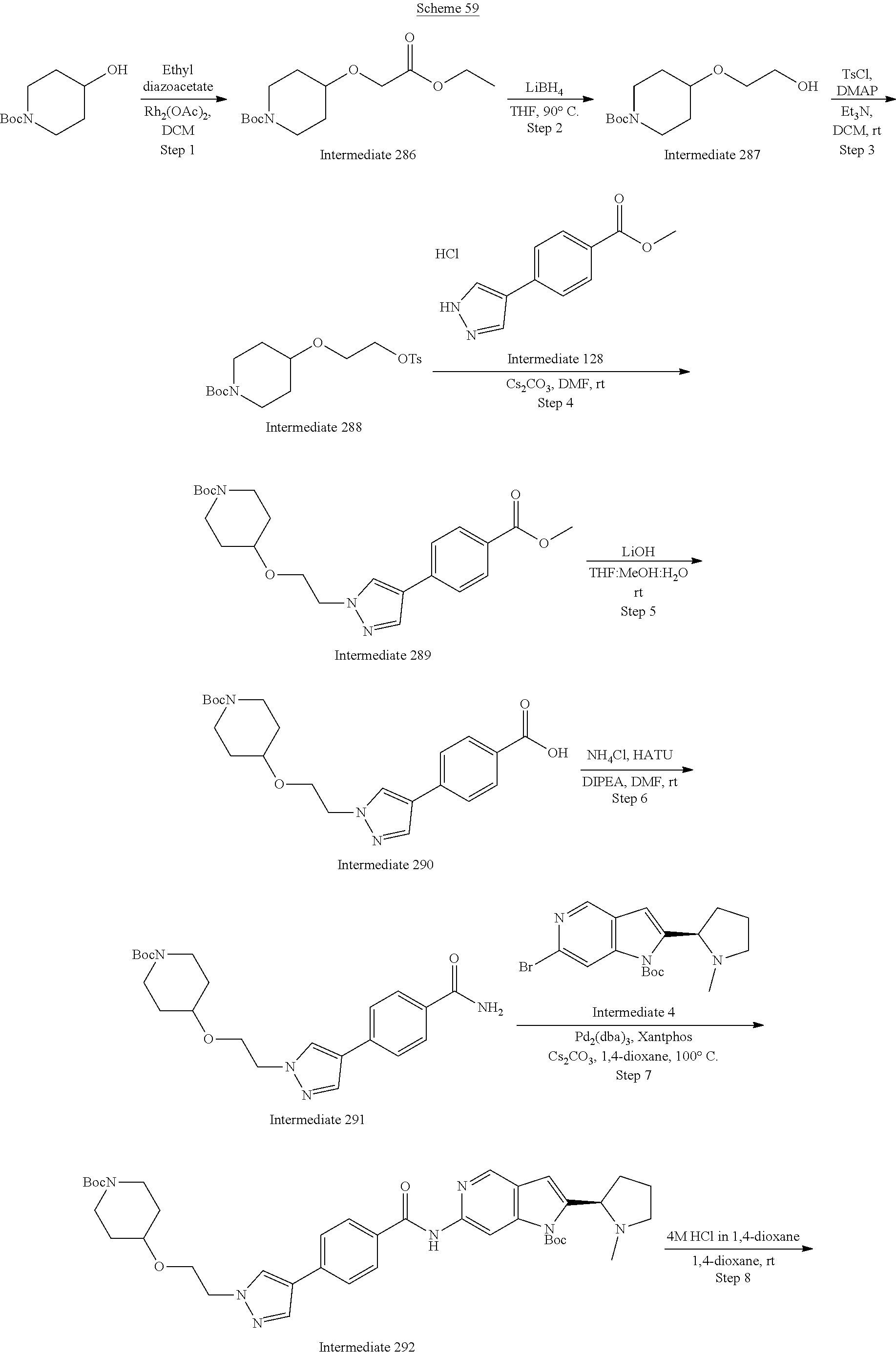

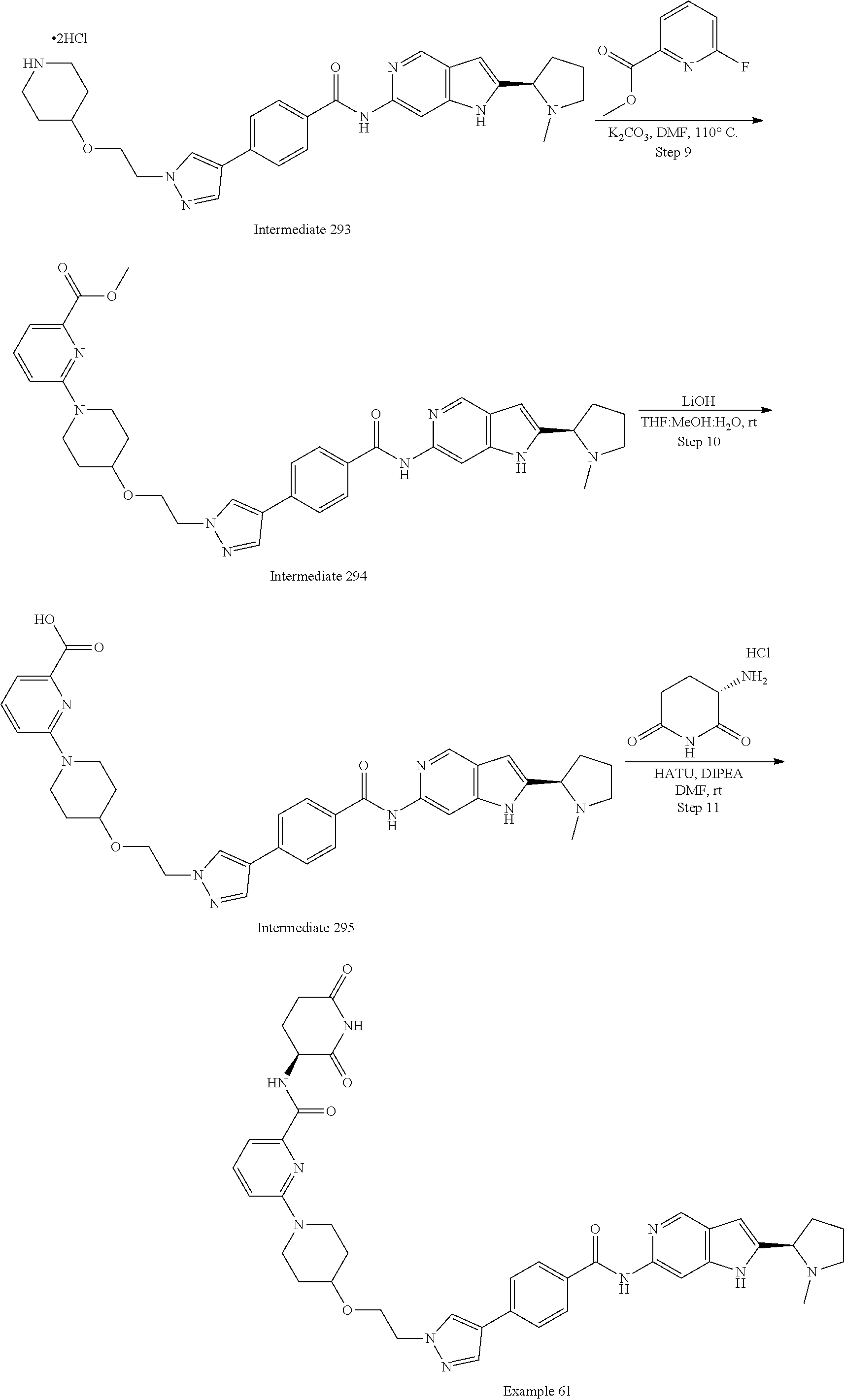

### Step 1

### Intermediate 286: tert-butyl-4-(2-ethoxy-2-oxoethoxy)piperidine-1-carboxylate

To a solution of tert-butyl-4-hydroxypiperidine-1-carboxylate (CAS No: 109384-19-2, 5.00 g, 25 mmol) in dichloromethane (100 mL) was added rhodium (II) acetate dimer (0.55 g, 1.2 mmol). The reaction mixture was stirred at room temperature for 10 min, then ethyl-2-diazoacetate (8.5 g, 75 mmol) was added and the reaction stirred at room temperature for 48 h. The reaction mixture was poured into water (200 mL) and extracted with dichloromethane (3×100 mL). The combined organic layers were separated, dried over anhydrous Na₂SO₄, filtered, and concentrated in vacuo. The crude material was purified by combi-flash column chromatography, by eluting with 40% ethyl acetate in heptane, to afford tert-butyl-4-(2-ethoxy-2-oxoethoxy) piperidine-1-carboxylate (Intermediate 286, 4.50 g, 63%) as a colorless oil. ¹H NMR (400 MHz, DMSO-d₆) δ ppm 1.18 (t, J=7.02 Hz, 3H), 1.27-1.34 (m, 2H), 1.37 (s, 9H), 1.70-1.82 (m, 2H), 2.94-3.00 (m, 2H), 3.46-3.54 (m, 1H), 3.56-3.64 (m, 2H), 4.05-4.13 (m, 4H). Mass spec m/z 232.2 [M+H]⁺.

### Step 2

### Intermediate 287: tert-butyl-4-(2-hydroxyethoxy)piperidine-1-carboxylate

To a solution of tert-butyl 4-(2-ethoxy-2-oxoethoxy)piperidine-1-carboxylate (Intermediate 286, 4.50 g, 16.0 mmol) in tetrahydrofuran (100 mL) was added lithium borohydride (1.1 g, 47.0 mmol) and the reaction mixture was stirred at 90° C. for 16 h. The reaction mixture was then poured into water (250 mL) and extracted with ethyl acetate (3×250 mL). The combined organic layers were dried over anhydrous Na₂SO₄, filtered, and concentrated in vacuo to afford tert-butyl 4-(2-hydroxyethoxy)piperidine-1-carboxylate (Intermediate 287, 3.00 g, 78%) as a colorless oil, which was used for the next step without further purification. ¹H NMR (400 MHz, DMSO-d₆) δ ppm 1.23-1.32 (m, 2H), 1.36 (s, 9H), 1.72-1.78 (m, 2H), 2.92-3.01 (m, 2H), 3.37-3.49 (m, 5H), 3.57-3.66 (m, 2H), 4.52-4.56 (m, 1H).

### Step 3

### Intermediate 288: tert-butyl 4-(2-(tosyloxy)ethoxy)piperidine-1-carboxylate

To a cooled (0° C.) solution of tert-butyl 4-(2-hydroxyethoxy) piperidine-1-carboxylate (Intermediate 287, 3.00 g, 12.0 mmol) in dichloromethane (200 mL) was added triethylamine (5.2 mL, 37 mmol) followed by p-toluenesulfonyl chloride (2.9 g, 15.0 mmol) and 4-dimethylaminopyridine (0.15 g, 1.2 mmol) and the reaction mixture was stirred at room temperature for 16 h. The reaction mixture was poured in water (200 mL) and extracted with dichloromethane (3×250 mL). The combined organic layers were dried over anhydrous Na₂SO₄, filtered, and concentrated in vacuo. The crude material was purified by combi-flash column chromatography, by eluting with 10% ethyl acetate in heptane, to afford tert-butyl-4-(2-(tosyloxy)ethoxy)piperidine-1-carboxylate (Intermediate 288, 4.00 g, 82%) as a colorless liquid. ¹H NMR (400 MHz, DMSO-d₆) δ ppm 1.18-1.24 (m, 2H), 1.39 (s, 9H), 1.62-1.68 (m, 2H), 2.42 (s, 3H), 2.92-2.98 (m, 2H), 3.36-3.42 (m, 1H), 3.46-3.63 (m, 4H), 4.08-4.16 (m, 2H), 7.47 (d, J=7.88 Hz, 2H), 7.78 (d, J=7.88 Hz, 2H). Mass spec m/z 399.9 [M+H]⁺.

### Step 4

### Intermediate 289: tert-butyl-4-(2-(4-(4-(methoxycarbonyl)phenyl)-1H-pyrazol-1-yl)ethoxy)piperidine-1-carboxylate

To a solution of tert-butyl 4-(2-(tosyloxy)ethoxy)piperidine-1-carboxylate (Intermediate 288, 4.00 g, 11.0 mmol) in dimethylformamide (25 mL) was added cesium carbonate (7.40 g, 23.0 mmol) followed by methyl 4-(1H-pyrazol-4-yl)benzoate hydrochloride (Intermediate 128, 1.80 g, 7.5 mmol) and the reaction mixture was stirred at room temperature for 12 h. The reaction mixture was poured in ice cold water (200 mL) and extracted with dichloromethane (3×100 mL). The combined organic layers were separated, dried over anhydrous Na₂SO₄, filtered, and concentrated in vacuo. The crude material was purified by combi-flash column chromatography, by eluting with 40% ethyl acetate in heptane, to afford tert-butyl 4-(2-(4-(4-(methoxycarbonyl)phenyl)-1H-pyrazol-1-yl) ethoxy) piperidine-1-carboxylate (Intermediate 289, 2.80 g, 86%) as a colorless oil. ¹H NMR (400 MHz, DMSO-d₆) δ ppm 1.24-1.30 (m, 2H), 1.35 (s, 9H), 1.64-1.72 (m, 2H), 2.94-3.04 (m, 2H), 3.42-3.52 (m, 3H), 3.78-3.82 (m, 2H), 3.84 (s, 3H), 4.24-4.28 (m, 2H), 7.72 (d, J=8.40 Hz, 2H), 7.92 (d, J=8.40 Hz, 2H), 7.95 (s, 1H), 8.31 (s, 1H). Mass spec m/z 430.0 [M+H]⁺.

### Step 5

### Intermediate 290: 4-(1-(2-((1-(tert-butoxycarbonyl)piperidin-4-yl)oxy)ethyl)-1H-pyrazol-4-yl)benzoic acid

To a solution of tert-butyl-4-(2-(4-(4-(methoxycarbonyl)phenyl)-1H-pyrazol-1-yl)ethoxy)piperidine-1-carboxylate (Intermediate 289, 2.80 g, 6.5 mmol) in tetrahydrofuran (25 mL), methanol (25 mL) and water (25 mL) was added lithium hydroxide (0.48 g, 20.0 mmol) and the reaction mixture was stirred at room temperature for 3 h then concentrated in vacuo. The obtained residue was taken up in water (10 mL) and acidified to pH~5 with 1N hydrochloric acid, then extracted with dichloromethane (3×100 mL). The combined organic layers were separated, dried over anhydrous Na₂SO₄, filtered, and concentrated in vacuo to afford 4-(1-(2-((1-(tert-butoxycarbonyl) piperidin-4-yl)oxy)ethyl)-1H-pyrazol-4-yl)benzoic acid (Intermediate 290, 2.30 g) as a colorless oil, which was used for the next step without further purification. ¹H NMR (400 MHz, DMSO-d₆) δ ppm 1.22-1.31 (m, 2H), 1.35 (s, 9H), 1.65-1.72 (m, 2H), 2.96-3.02 (m, 2H), 3.42-3.54 (m, 3H), 3.78-3.82 (m, 2H), 4.24-4.28 (m, 2H), 7.69 (d, J=7.89 Hz, 2H), 7.91 (d, J=7.89 Hz, 2H), 7.99 (s, 1H), 8.28 (s, 1H), 12.65 (br s, 1H). Mass spec m/z 415.9 [M+H]⁺.

### Step 6

### Intermediate 291: tert-butyl-4-(2-(4-(4-carbamoylphenyl)-1H-pyrazol-1-yl)ethoxy)piperidine-1-carboxylate

To a solution of 4-(1-(2-((1-(tert-butoxycarbonyl)piperidin-4-yl)oxy)ethyl)-1H-pyrazol-4-yl)benzoic acid (Intermediate 290, 2.30 g, 5.5 mmol) in dimethylformamide (25 mL) was added HATU (5.4 g, 14.0 mmol) and the reaction mixture stirred at room temperature for 15 min. Then ammonium chloride (1.5 g, 28.0 mmol) and N,N-diisopropylethylamine (2.9 mL, 17.0 mmol) were added and the reaction mixture was stirred at room temperature for 16 h. The reaction mixture was quenched with ice cold water (200 mL) and extracted with dichloromethane (2×200 mL). The combined organic layers were separated, washed with brine solution (100 mL), dried over anhydrous Na₂SO₄, filtered, and concentrated in vacuo. The crude material was purified by combi-flash column chromatography, by eluting with 5% MeOH in dichloromethane, to afford tert-butyl 4-(2-(4-(4-carbamoylphenyl)-1H-pyrazol-1-yl)ethoxy)piperidine-1-carboxylate (Intermediate 291, 2.0 g, 87%) as an off white solid. ¹H NMR (400 MHz, DMSO-d₆) δ ppm 1.35 (s, 9H), 1.66-1.74 (m, 2H), 3.11-3.18 (m, 2H), 3.42-3.53 (m, 3H), 3.58-3.64 (m, 2H), 3.78-3.82 (m, 2H), 4.24-4.28 (m, 2H), 7.27 (br s, 1H), 7.64 (d, J=8.00 Hz, 2H), 7.86 (d, J=8.00 Hz, 2H), 7.91 (br s, 1H), 7.97 (s, 1H), 8.25 (s, 1H). Mass spec m/z 415.0 [M+H]⁺.

### Step 7

### Intermediate 292: tert-butyl-(R)-6-(4-(1-(2-((1-(tert-butoxycarbonyl) piperidin-4-yl)oxy)ethyl)-1H-pyrazol-4-yl)benzamido)-2-(1-methylpyrrolidin-2-yl)-1H-pyrrolo[3,2-c]pyridine-1-carboxylate

To a solution of tert-butyl-4-(2-(4-(4-carbamoylphenyl)-1H-pyrazol-1-yl)ethoxy)piperidine-1-carboxylate (Intermediate 291, 1.50 g, 3.6 mmol) in 1,4-dioxane (20 mL) was added tert-butyl-(R)-6-bromo-2-(1-methylpyrrolidin-2-yl)-1H-pyrrolo[3,2-c]pyridine-1-carboxylate (Intermediate 4, 1.70 g, 4.47 mmol) followed by cesium carbonate (4.40 g, 13.5 mmol). The reaction mixture was purged with argon for 15 min then Pd₂(dba)₃ (0.63 g, 0.67 mmol) and Xantphos (0.80 g, 1.34 mmol) were added. The reaction mixture was purged with argon for another 10 min and the reaction mixture then stirred at 100° C. for 2 h. The reaction mixture was filtered through celite bed, the filter pad was washed with ethyl acetate (50 mL) and the filtrate was concentrated in vacuo. The crude material was purified by combi-flash column chromatography, by eluting with 90% ethyl acetate in heptane, to afford tert-butyl-(R)-6-(4-(1-(2-((1-(tert-butoxycarbonyl)piperidin-4-yl)oxy)ethyl)-1H-pyrazol-4-yl)benzamido)-2-(1-methylpyrrolidin-2-yl)-1H-pyrrolo[3,2-c]pyridine-1-carboxylate (Intermediate 292, 1.2 g, 38%) as a yellow solid. ¹H NMR (400 MHz, DMSO-d₆) δ ppm 1.22-1.32 (m, 2H), 1.36 (s, 9H), 1.54-1.64 (2H), 1.70 (s, 9H), 1.72-1.82 (m, 2H), 2.28-2.32 (m, 2H), 2.36 (s, 3H), 2.39-2.42 (m, 1H), 2.94-3.05 (m, 2H), 3.11-3.18 (m, 1H), 3.42-3.56 (m, 3H), 3.78-3.84 (m, 2H), 3.89-3.95 (m, 1H), 4.24-4.32 (m, 2H), 6.75 (s, 1H), 7.70 (d, J=8.0 Hz, 2H), 8.01 (s, 1H), 8.05 (d, J=8.0 Hz, 2H), 8.31 (s, 1H), 8.59 (s, 1H), 8.94 (s, 1H), 10.62 (br s, 1H). Mass spec m/z 714.0 [M+H]⁺.

### Step 8

### Intermediate 293: (R)—N-(2-(1-methylpyrrolidin-2-yl)-1H-pyrrolo[3,2-c]pyridin-6-yl)-4-(1-(2-(piperidin-4-yloxy)ethyl)-1H-pyrazol-4-yl)benzamide dihydrochloride

To a cooled (0° C.) solution of tert-butyl-(R)-6-(4-(1-(2-((1-(tert-butoxycarbonyl)piperidin-4-yl)oxy)ethyl)-1H-pyrazol-4-yl)benzamido)-2-(1-methylpyrrolidin-2-yl)-1H-pyrrolo[3,2-c]pyridine-1-carboxylate (Intermediate 292, 1.1 g, 1.5 mmol) in 1,4-dioxane (10 mL) was added 4M hydrochloric acid in 1,4-dioxane (20 mL) and the reaction mixture was stirred at room temperature for 3 h. The reaction mixture was concentrated in vacuo, the resultant solid was washed with ether (50 mL) and dried in vacuo to afford (R)—N-(2-(1-methylpyrrolidin-2-yl)-1H-pyrrolo[3,2-c]pyridin-6-yl)-4-(1-(2-(piperidin-4-yloxy)ethyl)-1H-pyrazol-4-yl)benzamide dihydrochloride (Intermediate 293, 0.80 g, 98%) as an off white solid which was used for the next step without further purification. ¹H NMR (400 MHz, DMSO-d₆) δ ppm 1.60-1.69 (m, 2H), 1.87-1.96 (m, 2H), 2.15-2.22 (m, 2H), 2.33-2.43 (m, 1H), 2.46 (s, 3H), 2.78-2.82 (m, 3H), 2.84-2.93 (m, 2H), 2.95-3.06 (m, 2H), 3.20-3.32 (m, 1H), 3.70-3.78 (m, 1H), 3.82-3.84 (m, 2H), 4.28-4.32 (m, 2H), 7.32 (s, 1H), 7.81-7.88 (m, 2H), 8.08 (s, 1H), 8.24-8.28 (m, 2H), 8.36-8.40 (m, 2H), 8.89 (br s, 1H), 9.04 (br s, 1H), 9.12 (s, 1H), 11.67 (br s, 1H), 12.19 (s, 1H), 13.46 (s, 1H).

### Step 9

### Intermediate 294: methyl-(R)-6-(4-(2-(4-(4-((2-(1-methylpyrrolidin-2-yl)-1H-pyrrolo[3,2-c]pyridin-6-yl)carbamoyl)phenyl)-1H-pyrazol-1-yl)ethoxy)piperidin-1-yl)picolinate

To a solution of of (R)—N-(2-(1-methylpyrrolidin-2-yl)-1H-pyrrolo[3,2-c]pyridin-6-yl)-4-(1-(2-(piperidin-4-yloxy)ethyl)-1H-pyrazol-4-yl)benzamide dihydrochoride (Intermediate 293, 0.80 g, 1.55 mmol) and methyl 6-fluoropicolinate (CAS No: 455-71-0, 0.24 g, 1.55 mmol) in dimethylformamide (10 mL) was added potassium carbonate (0.64 g, 4.63 mmol) and the reaction mixture was stirred at 110° C. for 16 h. The reaction mixture was then poured into water (100 mL), the resultant precipitate was collected by filtration and dried in vacuo to afford methyl-(R)-6-(4-(2-(4-(4-((2-(1-methylpyrrolidin-2-yl)-1H-pyrrolo[3,2-c]pyridin-6-yl)carbamoyl)phenyl)-1H-pyrazol-1-yl)ethoxy)piperidin-1-yl)picolinate (Intermediate 294, 0.51 g) as a brown solid, which was used for the next step without further purification. ¹H NMR (400 MHz, DMSO-d₆) δ ppm 1.33-1.47 (m, 2H), 1.72-1.96 (m, 5H), 2.05-2.12 (m, 1H), 2.17 (s, 3H), 2.22-2.32 (m, 1H), 2.52-2.56 (m, 1H), 3.10-3.23 (m, 3H), 3.35-3.43 (m, 1H), 3.50-3.60 (m, 1H), 3.81 (s, 3H), 3.85-3.94 (m, 3H), 4.26-4.32 (m, 2H), 6.39 (s, 1H), 7.02-7.05 (m, 1H), 7.24 (d, J=7.40 Hz, 1H), 7.62 (t, J=7.40 Hz, 1H), 7.69 (d, J=7.88 Hz, 2H), 8.01 (s, 1H), 8.04 (d, J=7.88 Hz, 2H), 8.20 (s, 1H), 8.31 (s, 1H), 8.50 (s, 1H), 10.36 (br s, 1H), 11.35 (br s, 1H). Mass spec m/z 648.9 [M+H]⁺.

### Step 10

### Intermediate 295: (R)-6-(4-(2-(4-(4-((2-(1-methylpyrrolidin-2-yl)-1H-pyrrolo[3,2-c]pyridin-6-yl) carbamoyl)phenyl)-1H-pyrazol-1-yl)ethoxy)piperidin-1-yl)picolinic acid

To a solution of methyl-(R)-6-(4-(2-(4-(4-((2-(1-methylpyrrolidin-2-yl)-1H-pyrrolo[3,2-c]pyridin-6-yl)carbamoyl)phenyl)-1H-pyrazol-1-yl)ethoxy)piperidin-1-yl)picolinate (Intermediate 294, 0.50 g, 0.77 mmol) in tetrahydrofuran (5 mL), methanol (5 mL) and water (2 mL) was added lithium hydroxide (0.05 g, 2.31 mmol) and the reaction mixture was stirred at room temperature for 16 h. The reaction mixture was concentrated in vacuo. The resultant residue was poured into ice cold water (100 mL), acidified with 1N hydrochloric acid to pH~5 and extracted with dichloromethane (3×100 mL). The combined organic layers were separated, dried over anhydrous Na₂SO₄, filtered, and concentrated in vacuo to afford (R)-6-(4-(2-(4-(4-((2-(1-methylpyrrolidin-2-yl)-1H-pyrrolo[3,2-c]pyridin-6-yl) carbamoyl)phenyl)-1H-pyrazol-1-yl) ethoxy) piperidin-1-yl) picolinic acid (Intermediate 295, 0.30 g, 61%) as a colorless oil, which was used for the next step without further purification. ¹H NMR (400 MHz, DMSO-d₆) δ ppm 1.37-1.50 (m, 2H), 1.77-1.90 (m, 5H), 2.09-2.14 (m, 1H), 2.17 (s, 3H), 2.19-2.29 (m, 1H), 3.11-3.25 (m, 2H), 3.34-3.36 (m, 2H), 3.49-3.54 (m, 1H), 3.86-3.88 (m, 2H), 3.91-4.01 (m, 2H), 4.28-4.32 (m, 2H), 6.39 (s, 1H), 6.88-6.94 (m, 1H), 7.18-7.20 (m, 1H), 7.56-7.71 (m, 3H), 7.99-8.07 (m, 3H), 8.20 (s, 1H), 8.32 (s, 1H), 8.51 (s, 1H), 10.39 (s, 1H), 11.37 (br s, 1H). Mass spec m/z 634.9 [M+H]⁺.

### Step 11

### Example 61: N—((S)-2,6-dioxopiperidin-3-yl)-6-(4-(2-(4-(4-((2-((R)-1-methylpyrrolidin-2-yl)-1H-pyrrolo[3,2-c]pyridin-6-yl)carbamoyl)phenyl)-1H-pyrazol-1-yl)ethoxy)piperidin-1-yl)picolinamide

To a solution of (R)-6-(4-(2-(4-(4-((2-(1-methylpyrrolidin-2-yl)-1H-pyrrolo[3,2-c]pyridin-6-yl) carbamoyl)phenyl)-1H-pyrazol-1-yl) ethoxy) piperidin-1-yl) picolinic acid (Intermediate 295, 0.30 g, 0.47 mmol) in dimethylformamide (5.0 mL) was added HATU (0.27 g, 0.70 mmol) and the mixture was stirred at room temperature for 15 min. (3S)-3-aminopiperidine-2,6-dione hydrochloride (CAS No: 25181-50-4, 0.15 g, 0.94 mmol) and N,N-diisopropylethylamine (0.30 g, 0.23 mmol) were added and the reaction was stirred at room temperature for a further 16 h. The reaction mixture was quenched with water (50 mL), the resultant precipitate was collected by filtration, washed with n-pentane (50 mL) and dried in vacuo. The crude material was purified by preparative HPLC (Method A) to afford N—((S)-2,6-dioxopiperidin-3-yl)-6-(4-(2-(4-(4-((2-((R)-1-methylpyrrolidin-2-yl)-1H-pyrrolo[3,2-c]pyridin-6-yl)carbamoyl)phenyl)-1H-pyrazol-1-yl)ethoxy)piperidin-1-yl)picolinamide (Example 61, 0.12 g, 29%) as an off white solid. ¹H NMR (400 MHz, DMSO-d₆) δ ppm 1.37-1.50 (m, 2H), 1.77-2.03 (m, 6H), 2.09-2.14 (m, 1H), 2.17 (s, 3H), 2.19-2.29 (m, 2H), 2.74-2.84 (m, 1H), 3.11-3.25 (m, 3H), 3.34-3.36 (m, 2H), 3.50-3.59 (m, 1H), 3.86-3.88 (m, 2H), 3.91-4.01 (m, 2H), 4.28-4.32 (m, 2H), 4.65-4.77 (m, 1H), 6.39 (s, 1H), 7.00-7.02 (m, 1H), 7.25-7.27 (m, 1H), 7.62-7.71 (m, 3H), 8.01 (s, 1H), 8.03-8.07 (m, 2H), 8.20 (s, 1H), 8.32 (s, 1H), 8.50 (s, 1H), 8.73 (d, J=8.38 Hz, 1H), 10.37 (s, 1H), 10.88 (s, 1H), 11.36 (s, 1H). Mass spec m/z 745.1 [M+H]⁺.

### Example 62 was Synthesised Following Scheme 60

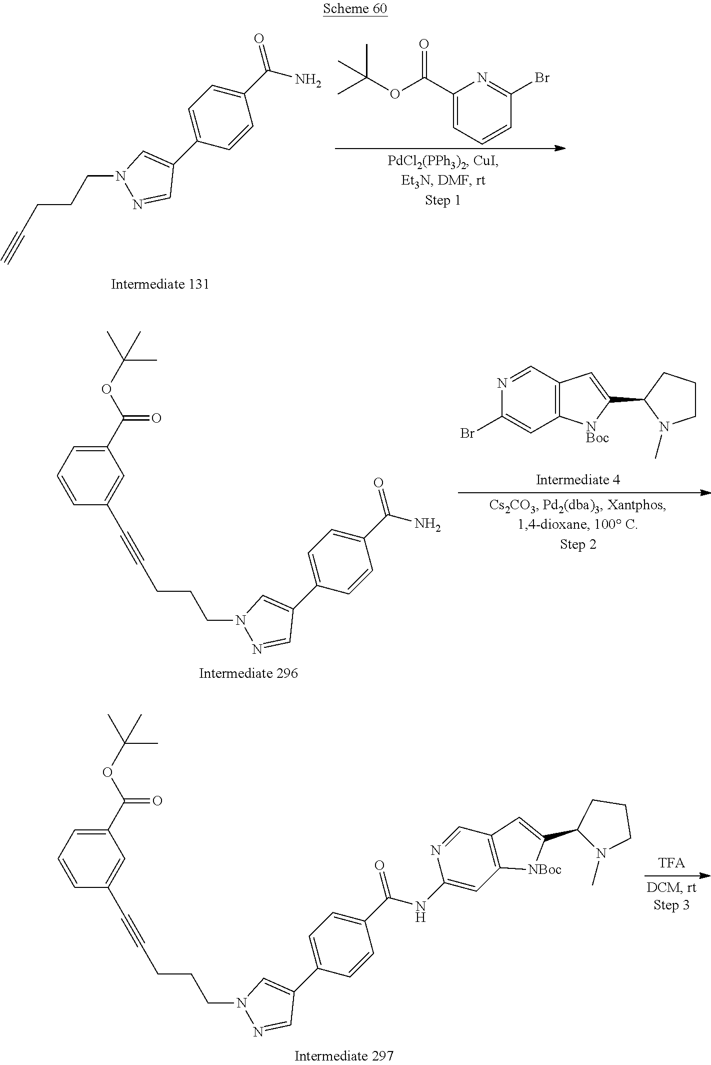

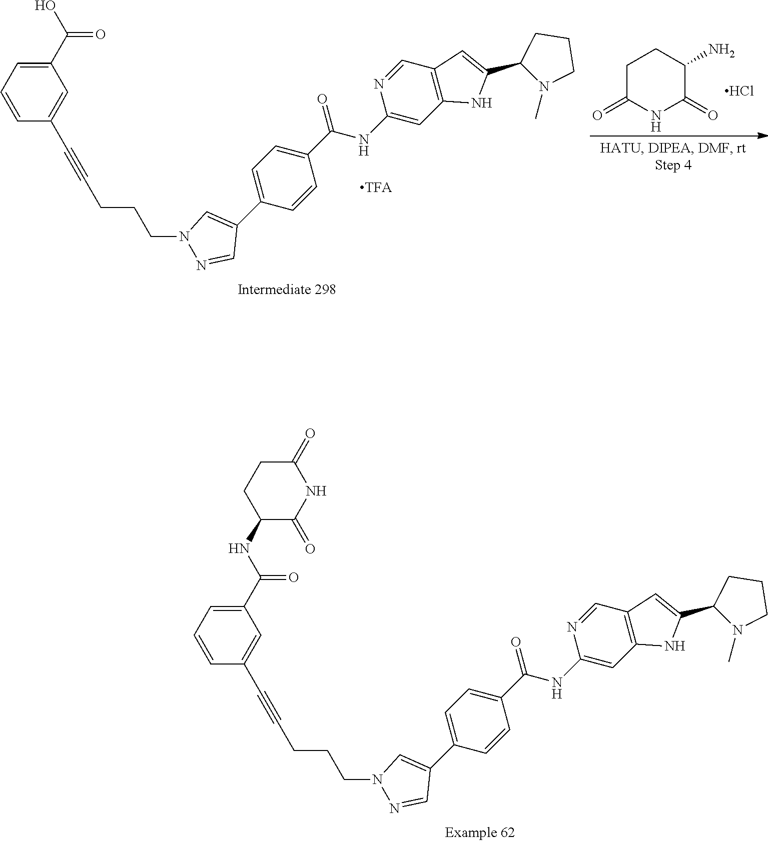 [SMILES: CN1CCC[C@@H]1c1cc2cnc(NC(=O)c3ccc(-c4cnn(CCCC#Cc5cccc(C(=O)O)c5)c4)cc3)cc2[nH]1 ; N[C@H]1CCC(=O)NC1=O ; Cl ; CN1CCC[C@@H]1c1cc2cnc(NC(=O)c3ccc(-c4cnn(CCCC#Cc5cccc(C(=O)N[C@H]6CCC(=O)NC6=O)c5)c4)cc3)cc2[nH]1]

### Step 1

### Intermediate 296: tert-butyl-6-(5-(4-(4-carbamoylphenyl)-1H-pyrazol-1-yl)pent-1-yn-1-yl) picolinate

To a solution of 4-(1-(pent-4-yn-1-yl)-1H-pyrazol-4-yl)benzamide (Intermediate 131, 1.50 g, 5.9 mmol) in dimethylformamide (2 mL) was added tert-butyl-6-bromopicolinate (CAS No: 910044-07-4, 1.50 g, 5.9 mmol) followed by triethylamine (31 mL, 220 mmol). The reaction mixture was purged with argon for 15 min, then copper (I) iodide (0.11 g, 0.59 mmol) and PdCl₂(PPh₃)₂ (0.43 g, 0.59 mmol) were added. The reaction mixture was further purged with argon for 10 min and stirred at room temperature for 2 h. The reaction mixture was then diluted with water (100 mL) and extracted with ethyl acetate (3×100 mL). The combined organic layers were separated, dried over anhydrous Na₂SO₄, and concentrated in vacuo. The crude material was purified by combi-flash chromatography, by eluting with 100% ethyl acetate in heptane, to afford tert-butyl-6-(5-(4-(4-carbamoylphenyl)-1H-pyrazol-1-yl)pent-1-yn-1-yl) picolinate (Intermediate 296, 1.80 g, 71%) as a white solid. ¹H NMR (400 MHz, DMSO-d₆) δ ppm 1.55 (s, 9H), 2.10-2.16 (m, 2H), 3.16 (t, J=6.40 Hz, 2H), 4.28 (t, J=6.40 Hz, 2H), 7.28 (br s, 1H), 7.63-7.67 (m, 3H), 7.85 (d, J=7.60 Hz, 2H), 7.90-7.95 (m, 3H), 7.99 (s, 1H), 8.34 (s, 1H).

### Step 2

### Intermediate 297: tert-butyl-(R)-6-(4-(1-(5-(6-(tert-butoxycarbonyl)pyridin-2-yl)pent-4-yn-1-yl)-1H-pyrazol-4-yl)benzamido)-2-(1-methylpyrrolidin-2-yl)-1H-pyrrolo[3,2-c]pyridine-1-carboxylate

To a solution of tert-butyl-6-(5-(4-(4-carbamoylphenyl)-1H-pyrazol-1-yl)pent-1-yn-1-yl) picolinate (Intermediate 296, 1.50 g, 3.5 mmol) in 1,4-dioxane (10 mL) was added tert-butyl-(R)-6-bromo-2-(1-methylpyrrolidin-2-yl)-1H-pyrrolo[3,2-c]pyridine-1-carboxylate (Intermediate 4, 1.30 g, 3.5 mmol) followed by cesium carbonate (3.40 g, 10 mmol). The reaction mixture was purged with argon for 15 min, then Pd₂(dba)₃ (0.49 g, 0.52 mmol) and Xantphos (0.62 g, 1.0 mmol) were added. The reaction mixture was purged with argon for another 10 min and then heated at 100° C. for 2 h. The reaction mixture was concentrated in vacuo and the crude material was purified by combi-flash chromatography, by eluting with 80% ethyl acetate in heptane, to afford tert-butyl-(R)-6-(4-(1-(5-(6-(tert-butoxycarbonyl)pyridin-2-yl)pent-4-yn-1-yl)-1H-pyrazol-4-yl)benzamido)-2-(1-methylpyrrolidin-2-yl)-1H-pyrrolo[3,2-c]pyridine-1-carboxylate (Intermediate 297, 1.00 g, 40%) as a white solid. ¹H NMR (400 MHz, DMSO-d₆) δ ppm 1.56 (s, 9H), 1.70 (s, 9H), 1.74-1.79 (m, 3H), 2.12-2.19 (m, 2H), 2.29-2.33 (m, 1H), 2.36 (s, 3H), 2.37-2.39 (m, 1H), 2.66-2.68 (m, 2H), 3.11-3.15 (m, 1H), 3.90-3.96 (m, 1H), 4.30 (t, J=6.40 Hz, 2H), 6.75 (s, 1H), 7.66-7.72 (m, 3H), 7.90-7.96 (m, 2H), 8.04-8.08 (m, 3H), 8.40 (s, 1H), 8.59 (s, 1H), 8.94 (s, 1H), 10.62 (br s, 1H). Mass spec m/z 730.56 [M+H]⁺.

### Step 3

### Intermediate 298: (R)-6-(5-(4-(4-((2-(1-methylpyrrolidin-2-yl)-1H-pyrrolo[3,2-c]pyridin-6-yl)carbamoyl)phenyl)-1H-pyrazol-1-yl)pent-1-yn-1-yl) picolinic acid trifluoroacetate

To a cooled (0° C.) solution of tert-butyl-(R)-6-(4-(1-(5-(6-(tert-butoxycarbonyl) pyridin-2-yl)pent-4-yn-1-yl)-1H-pyrazol-4-yl)benzamido)-2-(1-methylpyrrolidin-2-yl)-1H-pyrrolo[3,2-c]pyridine-1-carboxylate (Intermediate 297, 0.70 g, 0.96 mmol) in dichloromethane (5 mL) was added trifluoroacetic acid (5 mL) and the reaction mixture was stirred at room temperature for 4 h. The reaction mixture was concentrated in vacuo to afford (R)-6-(5-(4-(4-((2-(1-methylpyrrolidin-2-yl)-1H-pyrrolo[3,2-c]pyridin-6-yl)carbamoyl)phenyl)-1H-pyrazol-1-yl)pent-1-yn-1-yl) picolinic acid trifluoroacetate (Intermediate 298, 0.80 g) as a white solid which was used for the next step without further purification. Mass spec m/z 574.30 [M+H]⁺.

### Step 4

### Example 62: N—((S)-2,6-dioxopiperidin-3-yl)-6-(5-(4-(4-((2-((R)-1-methylpyrrolidin-2-yl)-1H-pyrrolo[3,2-c]pyridin-6-yl)carbamoyl)phenyl)-1H-pyrazol-1-yl)pent-1-yn-1-yl) picolinamide

To a solution of (R)-6-(5-(4-(4-((2-(1-methylpyrrolidin-2-yl)-1H-pyrrolo[3,2-c]pyridin-6-yl)carbamoyl)phenyl)-1H-pyrazol-1-yl)pent-1-yn-1-yl) picolinic acid trifluoroacetate (Intermediate 298, 0.60 g, 0.98 mmol) in dimethylformamide (5 mL) was added HATU (0.56 g, 0.15 mmol) and the reaction mixture was stirred at room temperature for 15 min. (S)-3-aminopiperidine-2,6-dione hydrochloride (CAS No: 25181-50-4, 0.16 g, 0.98 mmol) and N,N-diisopropylethylamine (0.3 mL, 1.96 mmol) were then added and the reaction mixture was stirred at room temperature for 16 h. The reaction mixture was then quenched with ice cold water (50 mL), the resultant precipitate was collected by filtration and dried in vacuo. The crude material was purified by preparative HPLC (Method A) to afford N—((S)-2,6-dioxopiperidin-3-yl)-6-(5-(4-(4-((2-((R)-1-methylpyrrolidin-2-yl)-1H-pyrrolo[3,2-c]pyridin-6-yl)carbamoyl)phenyl)-1H-pyrazol-1-yl)pent-1-yn-1-yl) picolinamide (Example 62, 0.50 g, 74%) as an off white solid. ¹H NMR (400 MHz, DMSO-d₆) δ ppm 1.76-2.01 (m, 5H), 2.12-2.16 (m, 2H), 2.17 (s, 3H), 2.19-2.21 (m, 1H), 2.23-2.30 (m, 2H), 2.53-2.57 (m, 2H), 2.75-2.85 (m, 1H), 3.12-3.16 (m, 1H), 3.32-3.35 (m, 1H), 4.30 (t, J=6.80 Hz, 2H), 4.77-4.84 (m, 1H), 6.39 (s, 1H), 7.69-7.73 (m, 3H), 7.98 (s, 1H), 7.99-8.02 (m, 1H), 8.04-8.06 (m, 2H), 8.07 (s, 1H), 8.20 (s, 1H), 8.39 (s, 1H), 8.50 (s, 1H), 8.97 (d, J=8.40 Hz, 1H), 10.39 (br s, 1H), 10.87 (br s, 1H), 11.36 (br s, 1H). Mass spec m/z 684.2 [M+H]⁺.

### Example 63 was Synthesised Following Scheme 61

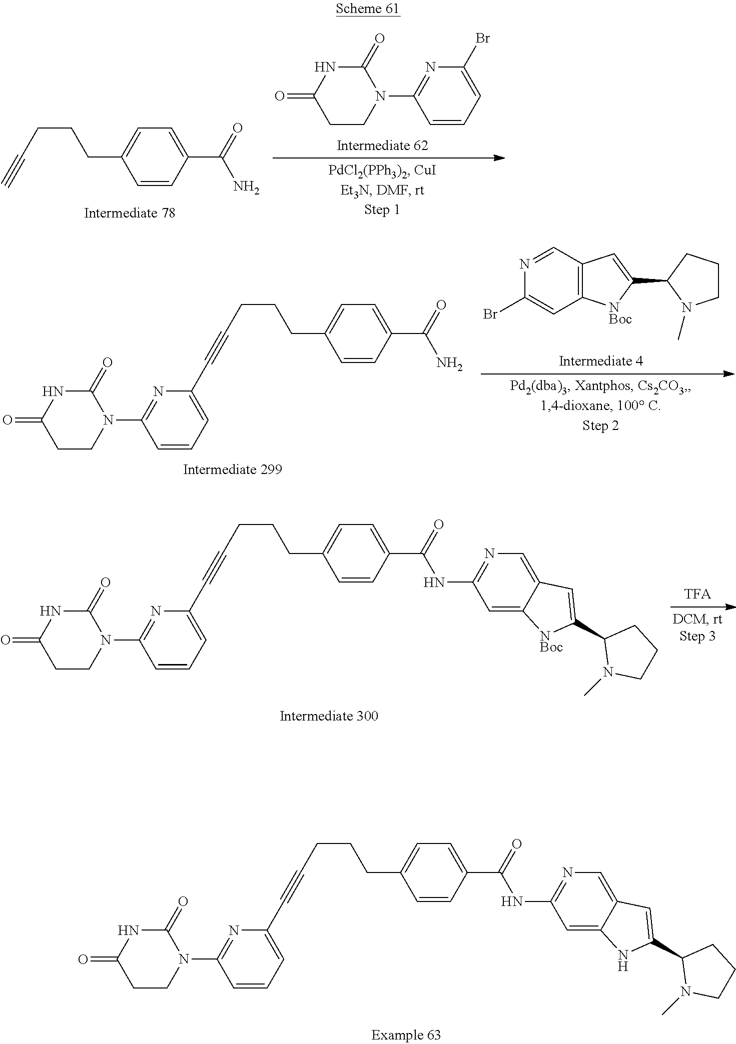 [SMILES: O=C1CCN(c2cccc(Br)n2)C(=O)N1 ; C#CCCCc1ccc(C(N)=O)cc1 ; NC(=O)c1ccc(CCCC#Cc2cccc(N3CCC(=O)NC3=O)n2)cc1 ; CN1CCC[C@@H]1c1cc2cnc(Br)cc2n1C(=O)OC(C)(C)C ; CN1CCC[C@@H]1c1cc2cnc(NC(=O)c3ccc(CCCC#Cc4cccc(N5CCC(=O)NC5=O)n4)cc3)cc2n1C(=O)OC(C)(C)C ; CN1CCC[C@@H]1c1cc2cnc(NC(=O)c3ccc(CCCC#Cc4cccc(N5CCC(=O)NC5=O)n4)cc3)cc2[nH]1]

### Step 1

### Intermediate 299: 4-(5-(6-(2,4-dioxotetrahydropyrimidin-1(2H)-yl)pyridin-2-yl)pent-4-yn-1-yl)benzamide

To a solution of 4-(pent-4-yn-1-yl)benzamide (Intermediate 78, 1.00 g, 3.70 mmol) in dimethylformamide (15 mL) was added 1-(6-bromopyridin-2-yl)dihydropyrimidine-2,4(1H,3H)-dione (Intermediate 62, 0.55 g, 2.96 mmol) followed by triethylamine (18.7 mL, 133.29 mmol). The reaction mixture was purged with argon for 15 min, then copper (I) iodide (0.072 g, 0.37 mmol) and PdCl₂(PPh₃)₂ (0.26 g, 0.37 mmol) were added. The reaction mixture was purged with argon for 10 min and then stirred at room temperature for 16 h. The reaction mixture was diluted with water (200 mL) and extracted with ethyl acetate (3×90 mL). The combined organic layers were dried over anhydrous Na₂SO₄, and concentrated in vacuo. The crude material was purified by combi-flash column chromatography, by eluting with 80% ethyl acetate in heptane, to afford 4-(5-(6-(2,4-dioxotetrahydropyrimidin-1(2H)-yl)pyridin-2-yl)pent-4-yn-1-yl)benzamide (Intermediate 299, 0.85 g, 61%) as a yellow solid. ¹H NMR (400 MHz, DMSO-d₆) δ ppm 1.84-1.93 (m, 2H), 2.44-2.48 (m, 2H), 2.66-2.70 (m, 2H), 2.76-2.80 (m, 2H), 4.01-4.04 (m, 2H), 7.25 (br s, 1H), 7.27-7.35 (m, 3H), 7.72-7.84 (m, 4H), 7.89 (br s, 1H), 10.55 (s, 1H). Mass spec m/z 377.2 [M+H]⁺.

### Step 2

### Intermediate 300: tert-butyl-(R)-6-(4-(5-(6-(2,4-dioxotetrahydropyrimidin-1(2H)-yl) pyridin-2-yl) pent-4-yn-1-yl)benzamido)-2-(1-methylpyrrolidin-2-yl)-1H-pyrrolo[3,2-c]pyridine-1-carboxylate

To a solution of 4-(5-(6-(2,4-dioxotetrahydropyrimidin-1(2H)-yl)pyridin-2-yl)pent-4-yn-1-yl)benzamide (Intermediate 299, 0.39 g, 1.05 mmol) in 1,4-dioxane (15 mL) was added tert-butyl-(R)-6-bromo-2-(1-methylpyrrolidin-2-yl)-1H-pyrrolo[3,2-c]pyridine-1-carboxylate (Intermediate 4, 0.5 g, 1.31 mmol) followed by cesium carbonate (1.28 g, 3.94 mmol). The reaction mixture was purged with argon for 15 min, then Pd₂(dba)₃ (0.18 g, 0.19 mmol) and Xantphos (0.23 g, 0.39 mmol) were added. The reaction mixture was purged with argon for another 10 min and heated at 100° C. for 2 h. The reaction mixture was filtered through celite bed and the filter pad was washed with ethyl acetate (100 mL). The filtrate was concentrated in vacuo and the crude material was purified by combi-flash chromatography, by eluting with 90% ethyl acetate in heptane, to afford tert-butyl-(R)-6-(4-(5-(6-(2,4-dioxotetrahydropyrimidin-1(2H)-yl)pyridin-2-yl)pent-4-yn-1-yl)benzamido)-2-(lmethylpyrrolidin-2-yl)-1H-pyrrolo[3,2-c]pyridine-1-carboxylate (Intermediate 300, 0.50 g, 56%) as a yellow solid. ¹H NMR (400 MHz, DMSO-d₆) δ ppm 1.70 (s, 9H), 1.74-1.80 (m, 3H), 1.85-1.96 (m, 2H), 2.28-2.32 (m, 1H), 2.35 (s, 3H), 2.39-2.42 (m, 1H), 2.52-2.56 (m, 2H), 2.66-2.70 (m, 2H), 2.76-2.86 (m, 2H), 3.12-3.16 (m, 1H), 3.90-3.94 (m, 1H), 4.02-4.10 (m, 2H), 6.75 (s, 1H), 7.27-7.33 (m, 2H), 7.38 (d, J=8.0 Hz, 1H), 7.67-7.84 (m, 3H), 7.99 (d, J=8.0 Hz, 1H), 8.59 (s, 1H), 8.93 (s, 1H), 10.55 (br s, 1H), 10.62 (br s, 1H). Mass spec m/z 576.3 [M−100+H]⁺.

### Step 3

### Example 63: (R)-4-(5-(6-(2,4-dioxotetrahydropyrimidin-1(2H)-yl)pyridin-2-yl)pent-4-yn-1-yl)-N-(2-(1-methylpyrrolidin-2-yl)-1H-pyrrolo[3,2-c]pyridin-6-yl)benzamide

To a cooled (0° C.) solution of tert-butyl-(R)-6-(4-(5-(6-(2,4-dioxotetrahydropyrimidin-1(2H)-yl) pyridin-2-yl) pent-4-yn-1-yl)benzamido)-2-(1-methylpyrrolidin-2-yl)-1H-pyrrolo[3,2-c]pyridine-1-carboxylate (Intermediate 300, 0.50 g, 0.74 mmol) in dichloromethane (10 mL) was added trifluoroacetic acid (5 mL) and the reaction mixture was stirred at room temperature for 1 h. The reaction mixture was concentrated in vacuo and the crude material was purified by preparative HPLC (Method A) to afford (R)-4-(5-(6-(2,4-dioxotetrahydropyrimidin-1(2H)-yl)pyridin-2-yl)pent-4-yn-1-yl)-N-(2-(1-methylpyrrolidin-2-yl)-1H-pyrrolo[3,2-c]pyridin-6-yl)benzamide (Example 63, 0.067 g, 16%) as a pale yellow solid. ¹H NMR (400 MHz, DMSO-d₆) δ ppm 1.75-1.98 (m, 6H), 2.17 (s, 3H), 2.21-2.24 (m, 1H), 2.25-2.30 (m, 1H), 2.45-2.47 (m, 2H), 2.64-2.72 (m, 2H), 2.79-2.84 (m, 2H), 3.11-3.21 (m, 1H), 4.02-4.06 (m, 2H), 6.40 (s, 1H), 7.26-7.30 (m, 1H), 7.35 (d, J=8.40 Hz, 2H), 7.70-7.74 (m, 1H), 7.76-7.80 (m, 1H), 8.00 (d, J=8.40 Hz, 2H), 8.19 (s, 1H), 8.50 (s, 1H), 10.38 (br s, 1H), 10.55 (br s, 1H), 11.37 (br s, 1H). Mass spec m/z 576.2 [M+H]⁺.

### Example 64 was Synthesised Following Scheme 62

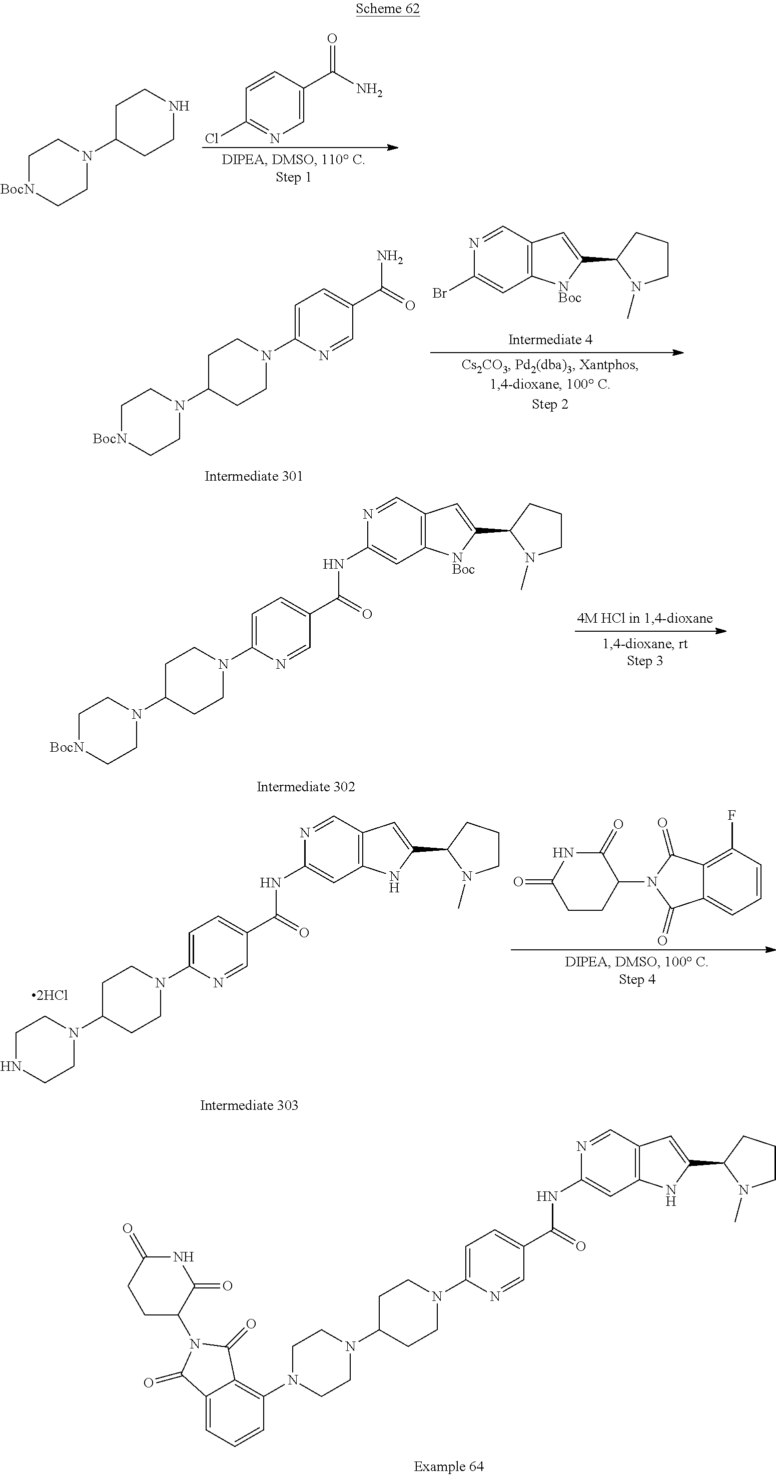

### Step 1

### Intermediate 301: tert-butyl-4-(1-(5-carbamoylpyridin-2-yl) piperidin-4-yl)piperazine-1-carboxylate

To a solution of tert-butyl 4-(piperidin-4-yl)piperazine-1-carboxylate (CAS No: 205059-24-1, 2.8 g, 11 mmol) in dimethyl sulfoxide (10 mL) was added 6-chloronicotinamide (CAS No: 6271-78-9, 1.5 g, 9.6 mmol) and the reaction mixture was heated at 110° C. for 2 h. The reaction mixture was poured into water (200 mL), the resultant precipitate was collected by filtration and dried in vacuo to afford tert-butyl 4-(1-(5-carbamoylpyridin-2-yl) piperidin-4-yl) piperazine-1-carboxylate (Intermediate 301, 1.00 g, 27%) as an off white solid, which was used for the next step without further purification. ¹H NMR (401 MHz, DMSO-d₆) δ ppm 1.30-1.36 (m, 2H), 1.39 (s, 9H), 1.76-1.85 (m, 2H), 2.40-2.46 (m, 4H), 2.54-2.58 (m, 1H), 2.82-2.88 (m, 2H), 3.22-3.32 (m, 4H), 4.38-4.48 (m, 2H), 6.84 (d, J=9.20 Hz, 1H), 7.12 (br s, 1H), 7.75 (br s, 1H), 7.93 (dd, J=9.20, 2.40 Hz, 1H), 8.59 (d, J=2.40 Hz, 1H). Mass spec m/z 390.27 [M+H]⁺.

### Step 2

### Intermediate 302: tert-butyl-(R)-6-(6-(4-(4-(tert-butoxycarbonyl)piperazin-1-yl)piperidin-1-yl) nicotinamido)-2-(1-methylpyrrolidin-2-yl)-1H-pyrrolo[3,2-c]pyridine-1-carboxylate

To a solution of tert-butyl-4-(1-(5-carbamoylpyridin-2-yl) piperidin-4-yl)piperazine-1-carboxylate (Intermediate 301, 0.41 g, 1.05 mmol) in 1,4-dioxane (15 mL) was added tert-butyl-(R)-6-bromo-2-(1-methylpyrrolidin-2-yl)-1H-pyrrolo[3,2-c]pyridine-1-carboxylate (Intermediate 4, 0.50 g, 1.31 mmol) followed by cesium carbonate (0.86 g, 2.63 mmol). The reaction mixture was purged with argon for 15 min then Pd₂(dba)₃ (0.18 g, 0.19 mmol) and Xantphos (0.23 g, 0.39 mmol) were added. The reaction mixture was purged with argon for another 10 min and then stirred at 100° C. for 2 h. The reaction mixture was concentrated in vacuo and the crude material was purified by combi-flash chromatography, by eluting with 80% ethyl acetate in heptane, to afford tert-butyl-(R)-6-(6-(4-(4-(tert-butoxycarbonylpiperazin-1-yl)piperidin-1-yl)nicotinamido)-2-(1-methylpyrrolidin-2-yl)-1H-pyrrolo[3,2-c]pyridine-1-carboxylate (Intermediate 302, 0.45 g, 50%) as a yellow solid. ¹H NMR (400 MHz, DMSO-d₆) δ ppm 1.39 (s, 9H), 1.54-1.62 (m, 2H), 1.69 (s, 9H), 1.73-1.87 (m, 4H), 2.28-2.33 (m, 1H), 2.35 (s, 3H), 2.38-2.47 (m, 6H), 2.86-2.94 (m, 2H), 3.11-318 (m, 1H), 3.23-3.27 (m, 3H), 3.32-3.36 (m, 2H), 3.88-3.95 (m, 1H), 4.44-4.48 (m, 2H), 6.74 (s, 1H), 6.88 (d, J=9.20 Hz, 1H), 8.13 (d, J=9.20 Hz, 1H), 8.57 (s, 1H), 8.78 (br s, 1H), 8.90 (s, 1H), 10.46 (br s, 1H). Mass spec m/z 689.57 [M+H]⁺.

### Step 3

### Intermediate 303: (R)—N-(2-(1-methylpyrrolidin-2-yl)-1H-pyrrolo[3,2-c]pyridin-6-yl)-6-(4-(piperazin-1-yl) piperidin-1-yl) nicotinamide dihydrochloride

To a cooled (0° C.) solution of tert-butyl-(R)-6-(6-(4-(4-(tert-butoxycarbonyl)piperazin-1-yl)piperidin-1-yl) nicotinamido)-2-(1-methylpyrrolidin-2-yl)-1H-pyrrolo[3,2-c]pyridine-1-carboxylate (Intermediate 302, 0.40 g, 0.58 mmol) in 1,4-dioxane (4 mL) was added 4M hydrochloric acid in 1,4-dioxane (6 mL) and the reaction mixture was stirred at room temperature for 16 h. The reaction mixture was concentrated in vacuo to afford (R)—N-(2-(1-methylpyrrolidin-2-yl)-1H-pyrrolo[3,2-c]pyridin-6-yl)-6-(4-(piperazin-1-yl)piperidin-1-yl)nicotinamide dihydrochloride (Intermediate 303, 0.40 g) as a brown solid, which was used for the next step without further purification. ¹H NMR (400 MHz, DMSO-d₆) δ ppm 1.66-1.78 (m, 3H), 2.16-2.28 (m, 5H), 2.49 (s, 3H), 2.96-3.04 (m, 2H), 3.23-3.27 (m, 3H), 3.52-3.56 (m, 2H), 3.62-3.76 (m, 7H), 4.66-4.76 (m, 2H), 7.12 (d, J=9.20 Hz, 1H), 7.28 (s, 1H), 8.23 (s, 1H), 8.36 (dd, J=2.40, 9.20 Hz, 1H), 8.99 (d, J=2.40 Hz, 1H), 9.08 (s, 1H), 9.83 (br s, 1H), 11.40 (br s, 1H), 12.01 (s, 1H), 12.16 (br s, 1H), 13.29 (br s, 1H). Mass spec m/z 489.37 [M+H]⁺.

### Step 4

### Example 64: 6-(4-(4-(2-(2,6-dioxopiperidin-3-yl)-1,3-dioxoisoindolin-4-yl)piperazin-1-yl)piperidin-1-yl)-N-(2-((R)-1-methylpyrrolidin-2-yl)-1H-pyrrolo[3,2-c]pyridin-6-yl)nicotinamide

To a solution of (R)—N-(2-(1-methylpyrrolidin-2-yl)-1H-pyrrolo[3,2-c]pyridin-6-yl)-6-(4-(piperazin-1-yl) piperidin-1-yl) nicotinamide dihydrochloride (Intermediate 303, 0.40 g, 0.8 mmol) in dimethyl sulfoxide (10 mL) was added 2-(2,6-dioxopiperidin-3-yl)-4-fluoroisoindoline-1,3-dione (CAS No: 835616-60-9, 0.20 g, 0.8 mmol) followed by N,N-diisopropylethylamine (0.7 mL, 4 mmol) and the reaction mixture was heated at 100° C. for 3 h. The reaction mixture was concentrated in vacuo. The obtained residue was poured into ice cold water (100 mL), the resultant precipitate was collected by filtration and dried in vacuo. The crude material was purified by preparative HPLC (Method A) to afford 6-(4-(4-(2-(2,6-dioxopiperidin-3-yl)-1,3-dioxoisoindolin-4-yl)piperazin-1-yl)piperidin-1-yl)-N-(2-((R)-1-methylpyrrolidin-2-yl)-1H-pyrrolo[3,2-c]pyridin-6-yl)nicotinamide (Example 64, 0.11 g, 20%) as an off-yellow solid. ¹H NMR (400 MHz, DMSO-d₆) δ ppm 1.38-1.51 (m, 2H), 1.76-1.96 (m, 5H), 1.99-2.06 (m, 1H), 2.09-2.14 (m, 1H), 2.16 (s, 3H), 2.22-2.30 (m, 1H), 2.39-2.44 (m, 2H), 2.55-2.63 (m, 3H), 2.66-2.73 (m, 4H), 2.82-2.99 (m, 4H), 3.11-3.17 (m, 1H), 3.25-3.29 (m, 2H), 4.44-4.52 (m, 2H), 5.06-5.12 (m, 1H), 6.37 (s, 1H), 6.90 (d, J=9.20 Hz, 1H), 7.30-7.37 (m, 2H), 7.68-7.72 (m, 1H), 8.13-8.17 (m, 2H), 8.48 (s, 1H), 8.79 (d, J=2.40 Hz, 1H), 10.26 (s, 1H), 11.08 (s, 1H), 11.33 (s, 1H). Mass spec m/z 745.3 [M+H]⁺.

### Example 65 was Synthesised Following Scheme 63

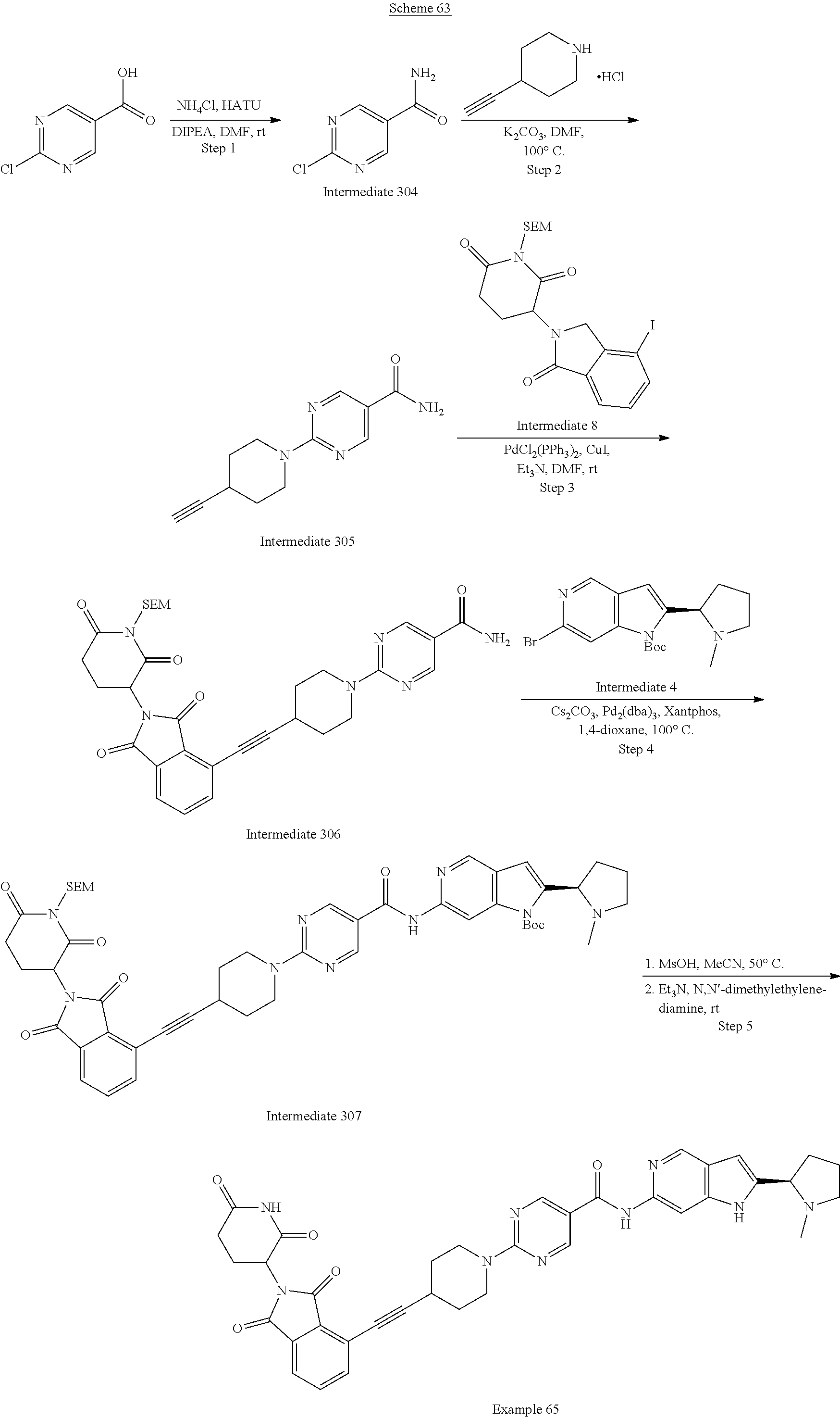

### Step 1

### Intermediate 304: 2-chloropyrimidine-5-carboxamide

To a solution of 2-chloropyrimidine-5-carboxylic acid (CAS No: 374068-01-6, 5.00 g, 31.54 mmol) in dimethylformamide (70 mL) was added HATU (18.93 g, 47.31 mmol) and N,N-diisopropylethylamine (16.5 mL, 94.61 mmol) and the reaction mixture was stirred at room temperature for 10 min. Ammonium chloride (6.75 g, 126.15 mmol) was added and the reaction mixture was stirred at room temperature for a further 16 h. The reaction mixture was quenched with ice cold water (200 mL), the resultant precipitate was collected by filtration and dried in vacuo to afford 2-chloropyrimidine-5-carboxamide (Intermediate 304, 5.10 g, 85%) as a white solid, which was used for the next step without further purification. ¹H NMR (400 MHz, DMSO-d₆) δ ppm 7.84 (br s, 1H), 8.26 (br s, 1H), 9.10 (s, 2H). Mass spec m/z 157.1 [M+H]⁺.

### Step 2

### Intermediate 305: 2-(4-ethynylpiperidin-1-yl)pyrimidine-5-carboxamide

To a solution of 4-ethynylpiperidine hydrochloride (CAS No: 550378-30-8, 1.80 g, 13 mmol) in dimethylformamide (20 mL) was added 2-chloropyrimidine-5-carboxamide (Intermediate 304, 2.00 g, 13 mmol) followed by potassium carbonate (7.0 g, 51 mmol) and the reaction mixture was heated at 100° C. for 2 h. The reaction mixture was quenched with water (50 mL) and extracted with ethyl acetate (2×50 mL). The combined organic layers were dried over anhydrous Na₂SO₄, filtered, and concentrated in vacuo. The crude material was purified by combi-flash chromatography, by eluting with 50% ethyl acetate in heptane, to afford 2-(4-ethynylpiperidin-1-yl)pyrimidine-5-carboxamide (Intermediate 305, 1.30 g, 43%) as an off-white solid. ¹H NMR (400 MHz, DMSO-d₆) δ ppm 1.44-1.55 (m, 2H), 1.78-1.87 (m, 2H), 2.70-2.79 (m, 1H), 3.01 (d, J=2.40 Hz, 1H), 3.45-3.56 (m, 2H), 4.14-4.26 (m, 2H), 7.29 (br s, 1H), 7.84 (br s, 1H), 8.77 (s, 2H). Mass spec m/z 231.5 [M+H]⁺.

### Step 3

### Intermediate 306: 2-(4-((2-(2,6-dioxo-1-((2-(trimethylsilyl)ethoxy)methyl)piperidin-3-yl)-1-oxoisoindolin-4-yl)ethynyl)piperidin-1-yl)pyrimidine-5-carboxamide

To a solution of 2-(4-ethynylpiperidin-1-yl)pyrimidine-5-carboxamide (Intermediate 305, 0.50 g, 2.17 mmol) in dimethylformamide (15 mL) was added 3-(4-iodo-1-oxoisoindolin-2-yl)-1-((2-(trimethylsilyl)ethoxy)methyl) piperidine-2,6-dione (Intermediate 8, 1.08 g, 2.17 mmol) followed by triethylamine (11.0 mL, 78.17 mmol). The reaction mixture was purged with argon for 15 min then copper (I) iodide (0.042 g, 0.22 mmol) and PdCl₂(PPh₃)₂ (0.15 g, 0.22 mmol) were added. The reaction mixture was further purged with argon for 10 min and stirred at room temperature for 16 h. The reaction mixture was diluted with water (200 mL) and extracted with ethyl acetate (3×90 mL). The combined organic layers were dried over anhydrous Na₂SO₄, filtered, and concentrated in vacuo. The crude material was purified by combi-flash chromatography, by eluting with 80% ethyl acetate in heptane, to afford 2-(4-((2-(2,6-dioxo-1-((2-(trimethylsilylethoxy)methylpiperidin-3-yl)-1-oxoisoindolin-4-yl) ethynyl)piperidin-1-yl)pyrimidine-5-carboxamide (Intermediate 306, 0.90 g, 69%) as a yellow solid. ¹H NMR (400 MHz, DMSO-d₆) δ ppm −0.05 (s, 9H), 0.77-0.88 (m, 2H), 1.58-1.70 (m, 2H), 1.88-2.00 (m, 2H), 2.02-2.10 (m, 1H), 2.39-2.47 (m, 1H), 2.76-2.82 (m, 1H), 3.01-3.12 (m, 2H), 3.48-3.64 (m, 4H), 4.22-4.31 (m, 3H), 4.46-4.52 (m, 1H), 5.00-5.10 (m, 2H), 5.24-5.30 (m, 1H), 7.27 (br s, 1H), 7.53 (t, J=8.0 Hz, 1H), 7.67 (d, J=7.20 Hz, 1H), 7.73 (d, J=7.20 Hz, 1H), 7.83 (br s, 1H), 8.77 (s, 2H). Mass spec m/z 603.1 [M+H]⁺.

### Step 4

### Intermediate 307: tert-butyl-6-(2-(4-((2-(2,6-dioxo-1-((2-(trimethylsilyl)ethoxy)methyl)piperidin-3-yl)-1-oxoisoindolin-4-yl)ethynyl)piperidin-1-yl)pyrimidine-5-carboxamido)-2-((R)-1-methylpyrrolidin-2-yl)-1H-pyrrolo[3,2-c]pyridine-1-carboxylate

To a solution of 2-(4-((2-(2,6-dioxo-1-((2-(trimethylsilyl) ethoxy)methyl) piperidin-3-yl)-1-oxoisoindolin-4-yl) ethynyl) piperidin-1-yl)pyrimidine-5-carboxamide (Intermediate 306, 0.79 g, 1.31 mmol) in 1,4-dioxane (10 mL) was added tert-butyl-(R)-6-bromo-2-(1-methylpyrrolidin-2-yl)-1H-pyrrolo[3,2-c]pyridine-1-carboxylate (Intermediate 4, 0.50 g, 1.31 mmol) followed by cesium carbonate (0.63 g, 3.29 mmol). The reaction mixture was purged with argon for 15 min then Pd₂(dba)₃ (0.12 g, 0.13 mmol) and Xantphos (0.23 g, 0.39 mmol) were added. The reaction mixture was further purged with argon for 10 min and heated at 100° C. for 2 h. The reaction mixture was concentrated in vacuo to obtain the crude compound. The crude material was purified by combi-flash chromatography, by eluting with 70% ethyl acetate in heptane, to afford tert-butyl-6-(2-(4-((2-(2,6-dioxo-1-((2-(trimethylsilyl)ethoxy)methyl)piperidin-3-yl)-1-oxoisoindolin-4-yl)ethynyl)piperidin-1-yl)pyrimidine-5-carboxamido)-2-((R)-1-methylpyrrolidin-2-yl)-1H-pyrrolo[3,2-c]pyridine-1-carboxylate (Intermediate 307, 0.80 g, 67%) as a yellow solid. ¹H NMR (400 MHz, DMSO-d₆) δ ppm −0.04 (s, 9H), 0.78-0.88 (m, 2H), 1.20-1.30 (m, 2H), 1.54-1.64 (m, 3H), 1.68 (s, 9H), 1.74-1.80 (m, 2H), 1.97-2.11 (m, 3H), 2.28-2.32 (m, 1H), 2.35 (s, 3H), 2.38-2.42 (m, 1H), 2.75-2.82 (m, 1H), 3.00-3.18 (m, 2H), 3.46-3.66 (m, 4H), 3.86-3.94 (m, 1H), 4.23-4.39 (m, 3H), 4.48-4.52 (m, 1H), 5.00-5.10 (m, 2H), 5.25-5.30 (m, 1H), 6.74 (s, 1H), 7.52-7.58 (m, 1H), 7.68 (d, J=7.60 Hz, 1H), 7.73 (d, J=7.60 Hz, 1H), 8.58 (s, 1H), 8.90 (s, 1H), 8.95 (s, 2H), 10.71 (br s, 1H). Mass spec m/z 901.9 [M+H]⁺.

### Step 5

### Example 65: 2-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)pyrimidine-5-carboxamide

To a solution of tert-butyl 6-(2-(4-((2-(2,6-dioxo-1-((2-(trimethylsilyl)ethoxy)methyl)piperidin-3-yl)-1-oxoisoindolin-4-yl)ethynyl)piperidin-1-yl)pyrimidine-5-carboxamido)-2-((R)-1-methylpyrrolidin-2-yl)-1H-pyrrolo[3,2-c]pyridine-1-carboxylate (Intermediate 307, 0.80 g, 0.88 mmol) in acetonitrile (10 mL) was added methanesulfonic acid (0.59 mL, 8.87 mmol) and the reaction mixture was heated at 50° C. for 2 h. The reaction mixture was cooled to room temperature, then N,N′-dimethylethylenediamine (0.53 mL, 4.43 mmol) followed by triethylamine (2.48 mL, 17.74 mmol) were added and the mixture stirred at room temperature for 3 h. The reaction mixture was concentrated in vacuo and the resultant residue was diluted with water (50 mL). The resultant precipitate was collected by filtration and dried in vacuo. The crude solid was purified by preparative HPLC (Method A) to afford 2-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)pyrimidine-5-carboxamide (Example 65, 0.18 g, 30%) as an off-white solid. ¹H NMR (400 MHz, DMSO-d₆) δ ppm 1.64-1.74 (m, 2H), 1.76-2.04 (m, 6H), 2.09-2.14 (m, 1H), 2.16 (s, 3H), 2.23-2.29 (m, 1H), 2.56-2.63 (m, 1H), 2.86-2.96 (m, 1H), 3.08-3.17 (m, 2H), 3.28-3.30 (m, 2H), 3.62-3.71 (m, 2H), 4.24-4.39 (m, 3H), 4.42-4.51 (m, 1H), 5.10-5.15 (m, 1H), 6.38 (s, 1H), 7.53 (t, J=7.60 Hz, 1H), 7.67 (dd, J=7.60, 1.20 Hz, 1H), 7.72 (dd, J=7.60, 0.80 Hz, 1H), 8.16 (s, 1H), 8.49 (s, 1H), 8.96 (s, 2H), 10.49 (s, 1H), 10.99 (br s, 1H), 11.37 (s, 1H). Mass spec m/z 672.1 [M+H]⁺.

### Example 66 was Synthesised Following Scheme 64

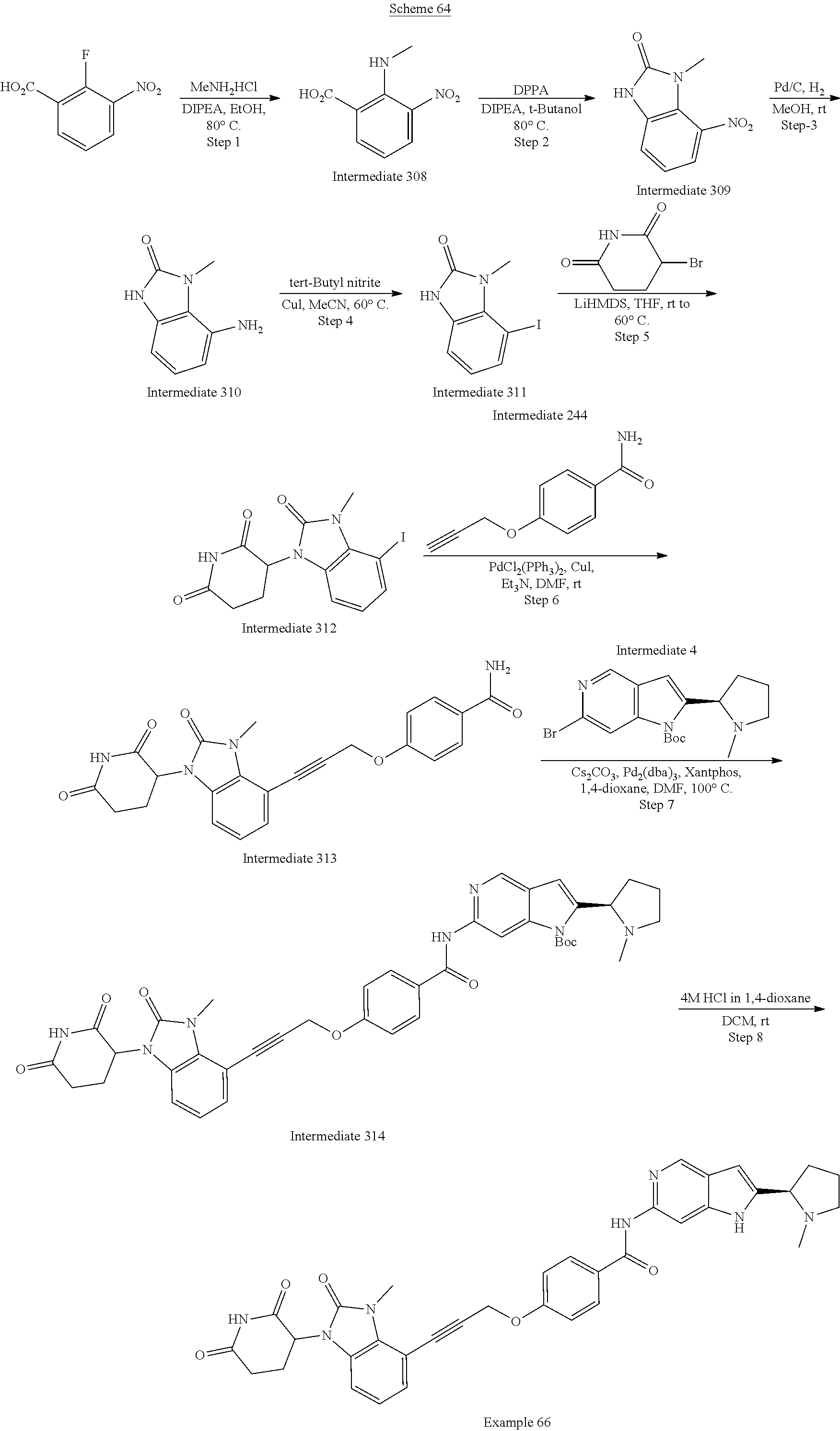

### Step 1

### Intermediate 308: 2-(methylamino)-3-nitrobenzoic acid

To a solution of 2-fluoro-3-nitro-benzoic acid (CAS No: 317-46-4, 20.0 g, 108.04 mmol) in ethanol (300 mL) was added methylamine hydrochloride (37.21 g, 540.22 mmol) followed by N,N-diisopropylethylamine (169.0 g, 1296.5 mmol). The reaction mixture was heated at 80° C. for 2 h then concentrated in vacuo. The obtained residue was diluted with water (100 mL) and acidified to pH~3 with 1N aqueous HCl. The resultant precipitate was collected by filtration and dried in vacuo to afford 2-(methylamino)-3-nitrobenzoic acid (Intermediate 308, 20.0 g, 94%) as a yellow solid, which was used for the next step without further purification. ¹H NMR (400 MHz, DMSO-d₆) δ ppm 2.69 (s, 3H), 6.72 (t, J=7.60 Hz, 1H), 7.98 (d, J=8.0 Hz, 1H), 8.04 (d, J=7.60 Hz, 1H), 8.61 (br s, 1H), 13.46 (br s, 1H). Mass spec m/z 197.0 [M+H]⁺.

### Step 2

### Intermediate 309: 1-methyl-7-nitro-1,3-dihydro-2H-benzo[d]imidazol-2-one

To a solution of 2-(methylamino)-3-nitrobenzoic acid (Intermediate 308, 20.0 g, 100.92 mmol) in tert-butanol (250 mL) was added N,N-diisopropylethylamine (26.3 g, 201.84 mmol) and diphenylphosphoryl azide (34.35 g, 121.10 mmol). The reaction mixture was heated at 80° C. for 2 h, then concentrated in vacuo. The obtained residue was diluted with water (100 mL), the resultant precipitate was collected by filtration and dried in vacuo to afford 1-methyl-7-nitro-1,3-dihydro-2H-benzo[d]imidazol-2-one (Intermediate 309, 15.0 g, 78%) as a yellow solid, which was used for the next step without further purification. ¹H NMR (400 MHz, DMSO-d₆) δ ppm 3.35 (s, 3H), 7.14 (t, J=8.0 Hz, 1H), 7.32 (d, J=7.60 Hz, 1H), 7.60 (d, J=8.0 Hz, 1H), 11.62 (br s, 1H). Mass spec m/z 192.0 [M−H]⁻.

### Step 3

### Intermediate 310: 7-amino-1-methyl-1,3-dihydro-2H-benzo[d]imidazol-2-one

To a solution of 1-methyl-7-nitro-1,3-dihydro-2H-benzo[d]imidazol-2-one (Intermediate 309, 8.0 g, 41.41 mmol) in methanol (350 mL) was added 10% Pd/C (3.00 g) and the reaction mixture was stirred under an atmosphere of hydrogen at 100 psi at room temperature for 16 h. The reaction mixture was filtered through celite bed and the filter pad was washed with methanol (100 mL). The filtrate was concentrated in vacuo to afford 7-amino-1-methyl-1,3-dihydro-2H-benzo[d]imidazol-2-one (Intermediate 310, 6.50 g, 96%) as a brown solid, which was used for the next step without further purification. ¹H NMR (400 MHz, DMSO-d₆) δ ppm 3.51 (s, 3H), 4.85 (br s, 2H), 6.30 (d, J=7.60 Hz, 1H), 6.35 (d, J=8.0 Hz, 1H), 6.68 (t, J=8.0 Hz, 1H), 10.54 (br s, 1H).

### Step 4

### Intermediate 311: 7-iodo-1-methyl-1,3-dihydro-2H-benzo[d]imidazol-2-one

To a solution of 7-amino-1-methyl-1,3-dihydro-2H-benzo[d]imidazol-2-one (Intermediate 310, 6.50 g, 40 mmol) in acetonitrile (120 mL) was added copper (I) iodide (12.0 g, 60.0 mmol). The reaction mixture was purged with argon for 20 min then tert-butyl nitrite (6.8 g, 60.0 mmol) was added and the reaction heated at 60° C. for 16 h. The reaction mixture was concentrated in vacuo and the crude material was purified by combi-flash chromatography, by eluting with 12% MeOH in dichloromethane, to afford 7-iodo-1-methyl-1,3-dihydro-2H-benzo[d]imidazol-2-one (Intermediate 311, 2.50 g, 23%) as a yellow solid. ¹H NMR (400 MHz, DMSO-d₆) δ ppm 3.56 (s, 3H), 6.75 (t, J=7.60 Hz, 1H), 6.98 (d, J=7.60 Hz, 1H), 7.38 (d, J=8.0 Hz, 1H), 11.07 (br s, 1H). Mass spec m/z 275.0 [M+H]⁺.

### Step 5

### Intermediate 312: 3-(4-iodo-3-methyl-2-oxo-2,3-dihydro-1H-benzo[d]imidazol-1-yl)piperidine-2,6-dione

To a solution of 7-iodo-1-methyl-1,3-dihydro-2H-benzo[d]imidazol-2-one (Intermediate 311, 2.50 g, 9.1 mmol) in tetrahydrofuran (20 mL) was added 1.0 M LiHMDS in tetrahydrofuran (23 mL, 23 mmol) dropwise and the reaction mixture was stirred at room temperature for 30 min. The above reaction mixture was then added to a solution of 3-bromopiperidine-2,6-dione (CAS No: 62595-74-8, 3.50 g, 18.0 mmol) in tetrahydrofuran (30 mL) and the reaction heated at 60° C. for 4 h. The reaction mixture was quenched with saturated aqueous ammonium chloride solution (50 mL) and the mixture stirred at room temperature for 30 min. The resultant precipitate was collected by filtration, washed with water (50 mL) and dried in vacuo to afford 3-(4-iodo-3-methyl-2-oxo-2,3-dihydro-1H-benzo[d]imidazol-1-yl)piperidine-2,6-dione (Intermediate 312, 0.51 g) as an off-white solid, which was used for the next step without further purification. ¹H NMR (400 MHz, DMSO-d₆) δ ppm 1.98-2.04 (m, 1H), 2.58-2.77 (m, 2H), 2.82-2.94 (m, 1H), 3.64 (s, 3H), 5.34-5.45 (m, 1H), 6.82 (t, J=7.60 Hz, 1H), 7.16 (d, J=7.60 Hz, 1H), 7.48 (d, J=7.60 Hz, 1H), 11.11 (br s, 1H). Mass spec m/z 386.06 [M+H]⁺.

### Step 6

### Intermediate 313: 4-((3-(1-(2,6-dioxopiperidin-3-yl)-3-methyl-2-oxo-2,3-dihydro-1H-benzo[d]imidazol-4-yl) prop-2-yn-1-yl)oxy)benzamide

To a solution of 3-(4-iodo-3-methyl-2-oxo-2,3-dihydro-1H-benzo[d]imidazol-1-yl)piperidine-2,6-dione (Intermediate 312, 0.45 g, 1.16 mmol) in dimethylformamide (5 mL) was added 4-(prop-2-yn-1-yloxy)benzamide (Intermediate 244, 0.30 g, 1.75 mmol) followed by triethylamine (6.1 mL, 43.22 mmol). The reaction mixture was purged with argon for 15 min, then copper (I) iodide (0.04 g, 0.23 mmol) and PdCl₂(PPh₃)₂ (0.08 g, 0.11 mmol) were added. The reaction mixture was purged with argon for another 10 min and then stirred at room temperature for 1 h. The reaction mixture was concentrated in vacuo and the crude material was purified by combi-flash chromatography, by eluting with 10% MeOH in dichloromethane, to afford 4-((3-(1-(2,6-dioxopiperidin-3-yl)-3-methyl-2-oxo-2,3-dihydro-1H-benzo[d]imidazol-4-yl) prop-2-yn-1-yl)oxy)benzamide (Intermediate 313, 0.21 g, 42%) as an off-white solid. ¹H NMR (400 MHz, DMSO-d₆) δ ppm 1.94-2.06 (m, 1H), 2.58-2.71 (m, 2H), 2.81-2.93 (m, 1H), 3.48 (s, 3H), 5.20 (s, 2H), 5.34-5.42 (m, 1H), 6.98-7.05 (m, 1H), 7.08-7.14 (m, 3H), 7.15-7.22 (m, 2H), 7.82-7.90 (m, 3H), 11.10 (s, 1H). Mass spec m/z 433.20 [M+H]⁺.

### Step 7

### Intermediate 314: tert-butyl-6-(4-((3-(1-(2,6-dioxopiperidin-3-yl)-3-methyl-2-oxo-2,3-dihydro-1H-benzo[d]imidazol-4-yl)prop-2-yn-1-yl)oxy)benzamido)-2-((R)-1-methylpyrrolidin-2-yl)-1H-pyrrolo[3,2-c]pyridine-1-carboxylate

To a solution of 4-((3-(1-(2,6-dioxopiperidin-3-yl)-3-methyl-2-oxo-2,3-dihydro-1H-benzo[d]imidazol-4-yl)prop-2-yn-1-yl)oxy)benzamide (Intermediate 313, 0.50 g, 1.15 mmol) in 1,4-dioxane (30 mL) and dimethylformamide (3 mL) was added tert-butyl-(R)-6-bromo-2-(1-methylpyrrolidin-2-yl)-1H-pyrrolo[3,2-c]pyridine-1-carboxylate (Intermediate 4, 0.52 g, 1.38 mmol) followed by cesium carbonate (1.14 g, 3.46 mmol). The reaction mixture was purged with argon for 15 min, then Pd₂(dba)₃ (0.16 g, 0.17 mmol) and Xantphos (0.20 g, 0.34 mmol) were added. The reaction mixture was further purged with argon for another 10 min and then heated at 100° C. for 2 h. The reaction mixture was concentrated in vacuo and the crude material was purified by combi-flash chromatography, by eluting with 11% MeOH in dichloromethane, to afford tert-butyl-6-(4-((3-(1-(2,6-dioxopiperidin-3-yl)-3-methyl-2-oxo-2,3-dihydro-1H-benzo[d]imidazol-4-yl)prop-2-yn-1-yl)oxy)benzamido)-2-((R)-1-methylpyrrolidin-2-yl)-1H-pyrrolo[3,2-c]pyridine-1-carboxylate (Intermediate 314, 0.40 g, 47%) as an off-white solid. ¹H NMR (400 MHz, DMSO-d₆) δ ppm 1.56-1.65 (m, 2H), 1.69 (s, 9H), 1.72-1.80 (m, 2H), 1.96-2.05 (m, 2H), 2.28-2.33 (m, 1H), 2.35 (s, 3H), 2.38-2.40 (m, 1H), 2.64-2.68 (m, 1H), 2.81-2.92 (m, 1H), 3.13-3.18 (m, 1H), 3.51 (s, 3H), 5.25 (s, 2H), 5.36-5.40 (m, 1H), 6.74 (s, 1H), 6.99-7.05 (m, 1H), 7.17 (d, J=8.80 Hz, 2H), 7.87 (d, J=8.80 Hz, 2H), 8.10 (d, J=8.80 Hz, 2H), (s, 1H), 8.92 (s, 1H), 10.60 (s, 1H), 11.11 (s, 1H). Mass spec m/z 732.45 [M+H]⁺.

### Step 8

### Example 66: 4-((3-(1-(2,6-dioxopiperidin-3-yl)-3-methyl-2-oxo-2,3-dihydro-1H-benzo[d]imidazol-4-yl)prop-2-yn-1-yl)oxy)-N-(2-((R)-1-methylpyrrolidin-2-yl)-1H-pyrrolo[3,2-c]pyridin-6-yl)benzamide

To a cooled (0° C.) solution of tert-butyl-6-(4-((3-(1-(2,6-dioxopiperidin-3-yl)-3-methyl-2-oxo-2,3-dihydro-1H-benzo[d]imidazol-4-yl)prop-2-yn-1-yl)oxy)benzamido)-2-((R)-1-methylpyrrolidin-2-yl)-1H-pyrrolo[3,2-c]pyridine-1-carboxylate (Intermediate 314, 0.40 g, 0.54 mmol) in dichloromethane (4 mL) was added 4M hydrochloric acid in 1,4-dioxane (2 mL) and the reaction mixture was stirred at room temperature for 4 h. The reaction mixture was concentrated in vacuo and the crude material was purified by preparative HPLC (Method A) to afford 4-((3-(1-(2,6-dioxopiperidin-3-yl)-3-methyl-2-oxo-2,3-dihydro-1H-benzo[d]imidazol-4-yl)prop-2-yn-1-yl)oxy)-N-(2-((R)-1-methylpyrrolidin-2-yl)-1H-pyrrolo[3,2-c]pyridin-6-yl)benzamide (Example 66, 0.10 g, 31%) as an off white solid. ¹H NMR (400 MHz, DMSO-d₆) δ ppm 1.75-1.95 (m, 3H), 1.98-2.04 (m, 1H), 2.09-2.14 (m, 1H), 2.17 (s, 3H), 2.23-2.30 (m, 1H), 2.58-2.71 (m, 3H), 2.81-2.92 (m, 1H), 3.12-3.16 (m, 1H), 3.51 (s, 3H), 5.25 (s, 2H), 5.36-5.40 (m, 1H), 6.38 (s, 1H), 7.03 (t, J=8.0 Hz, 1H), 7.11-7.14 (m, 1H), 7.15-7.20 (m, 3H), 8.06-8.11 (m, 2H), 8.18 (s, 1H), 8.49 (s, 1H), 10.35 (s, 1H), 11.10 (s, 1H), 11.34 (s, 1H). Mass spec m/z 632.2 [M+H]⁺.

### Example 67 was Synthesised Following Scheme 65

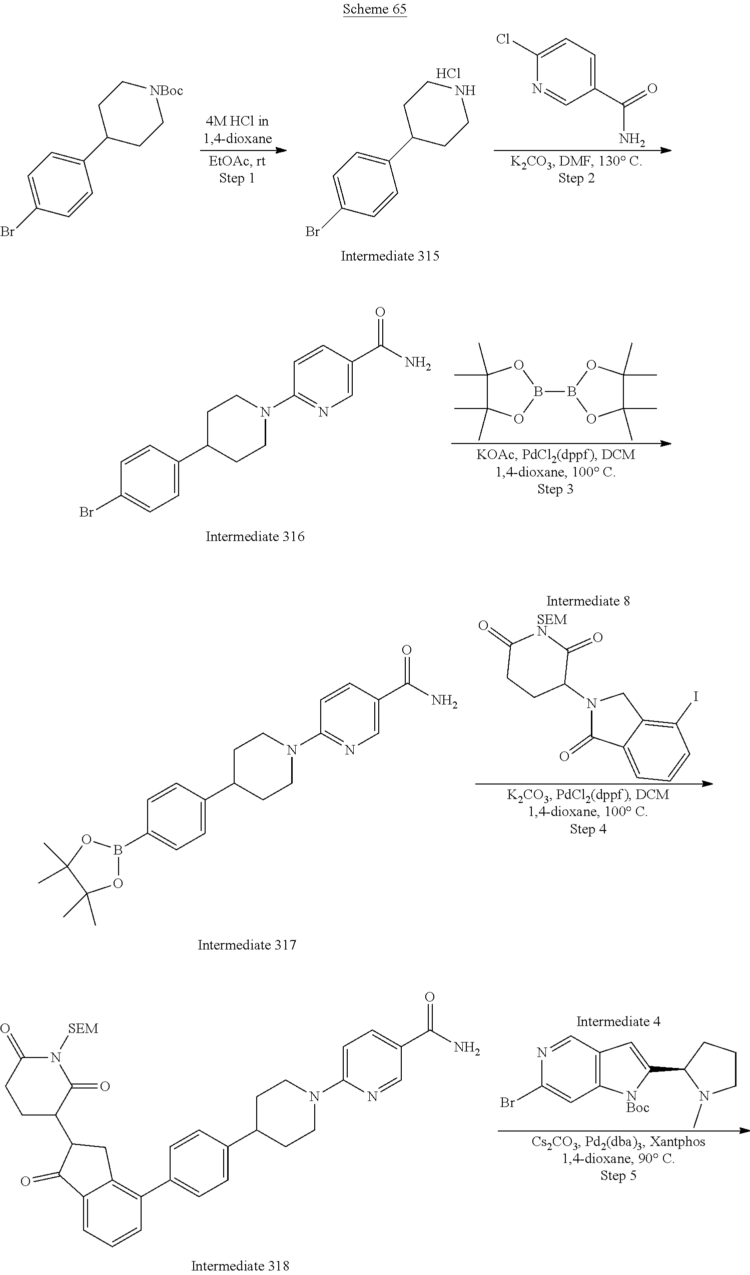

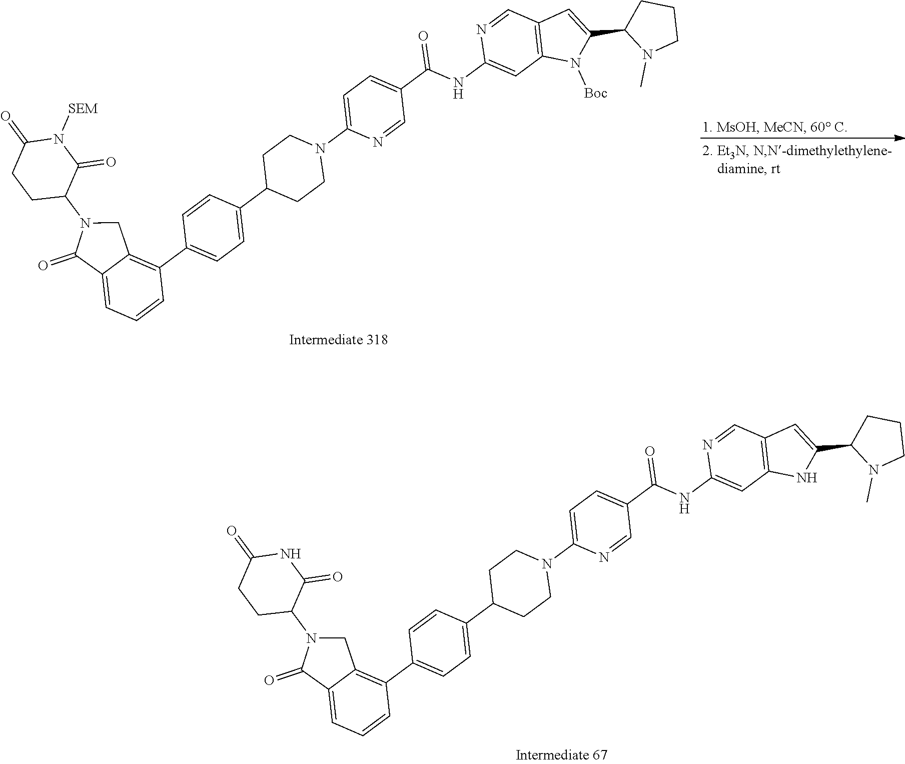 [SMILES: CN1CCC[C@@H]1c1cc2cnc(NC(=O)c3ccc(N4CCC(c5ccc(-c6cccc7c6CN(C6CCC(=O)N(COCC[Si](C)(C)C)C6=O)C7=O)cc5)CC4)nc3)cc2n1C(=O)OC(C)(C)C ; CN1CCC[C@@H]1c1cc2cnc(NC(=O)c3ccc(N4CCC(c5ccc(-c6cccc7c6CN(C6CCC(=O)NC6=O)C7=O)cc5)CC4)nc3)cc2[nH]1]

### Step 1

### Intermediate 315: 4-(4-bromophenyl)piperidine hydrochloride

To a solution of tert-butyl-4-(4-bromophenyl)piperidine-1-carboxylate (CAS No: 352437-09-3, 6.00 g, 17.6) in ethyl acetate (10 mL) was added 4M hydrochloric acid in 1,4-dioxane (20 mL) at 0° C. The reaction mixture was stirred at room temperature for 16 h. Progress of the reaction was monitored by TLC. After completion, the reaction mixture was concentrated in vacuo and washed with n-pentane (20 ml) to afford 4-(4-bromophenyl)piperidine hydrochloride (Intermediate 315, 4.60 g, 94%) as an off white solid, which was used for the next step without further purification. ¹H NMR (400 MHz, DMSO-d₆) δ ppm 1.75-1.94 (m, 4H), 2.77-2.88 (m, 1H), 2.90-3.02 (m, 2H), 3.28-3.34 (m, 2H), 7.13-7.25 (m, 2H), 7.45-7.59 (m, 2H), 8.87 (br s, 1H), 8.97 (br s, 1H). Mass spec m/z 240.3 [M+H]⁺.

### Step 2

### Intermediate 316: 6-(4-(4-bromophenyl) piperidin-1-yl)nicotinamide

To a solution of 4-(4-bromophenyl)piperidine hydrochloride (Intermediate 315, 4.00 g, 14.0 mmol) in dimethylformamide (30 mL) was added 6-chloronicotinamide (CAS No: 6271-78-9, 2.00 g, 13.0 mmol) followed by potassium carbonate (8.00 g, 58.0 mmol) and the reaction mixture was stirred at 130° C. for 16 h. The reaction mixture was poured into ice cold water (300 mL). The resultant precipitate was collected by filtration, washed with water (50 mL and dried in vacuo to afford 6-(4-(4-bromophenyl) piperidin-1-yl) nicotinamide (Intermediate 316, 4.50 g, 86%) as an off white solid, which was used for the next step without further purification. ¹H NMR (400 MHz, DMSO-d₆) δ ppm 1.51-1.63 (m, 2H), 1.75-1.88 (m, 2H), 2.78-2.86 (m, 1H), 2.91-3.00 (m, 2H), 4.50-4.62 (m, 2H), 6.86-6.88 (m, 1H), 7.08 (br s, 1H), 7.22 (d, J=8.0 Hz, 2H), 7.47 (d, J=8.0 Hz, 2H), 7.72 (br s, 1H), 7.92-7.96 (m, 1H), 8.62 (s, 1H). Mass spec m/z 360.1 [M+H]⁺.

### Step 3

### Intermediate 317: 6-(4-(4-(4,4,5,5-tetramethyl-1,3,2-dioxaborolan-2-ylphenyl) piperidin-1-yl) nicotinamide

To a solution of 6-(4-(4-bromophenyl)piperidin-1-yl)nicotinamide (Intermediate 316, 1.50 g, 4.2 mmol) 1,4-dioxane (30 mL) was added bis(pinacolato)diboron (CAS No: 73183-34-3, 1.40 g, 5.4 mmol) followed by potassium acetate (1.2 g, 12.0 mmol). The reaction mixture was purged with argon for 15 min then PdCl₂(dppf)·DCM (0.34 g, 0.42 mmol) was added. The reaction mixture was purged with argon for another 10 min and then stirred at 100° C. for 4 h. The reaction mixture was then concentrated in vacuo. The obtained solid was washed with diethyl ether (2×50 mL) and dried in vacuo to afford 6-(4-(4-(4,4,5,5-tetramethyl-1,3,2-dioxaborolan-2-yl)phenyl) piperidin-1-yl)nicotinamide (Intermediate 317, 3.60 g) as a grey solid, which was used for the next step without further purification. ¹H NMR (400 MHz, DMSO-d₆) δ ppm 1.15 (s, 6H), 1.27 (s, 6H), 1.50-1.65 (m, 2H), 1.81-1.86 (m, 2H), 2.83-3.00 (m, 3H), 4.50-4.58 (m, 2H), 6.86-6.88 (m, 1H), 7.10 (br s, 1H), 7.26 (d, J=7.60 Hz, 2H), 7.60 (d, J=7.60 Hz, 2H), 7.76 (br s, 1H), 7.92-7.96 (m, 1H), 8.62 (s, 1H). Mass spec m/z 408.2 [M+H]⁺.

### Step 4

### Intermediate 318: 6-(4-(4-(2-(2,6-dioxo-1-((2-(trimethylsilyl)ethoxy)methyl)piperidin-3-yl)-1-oxoisoindolin-4-yl)phenyl)piperidin-1-yl)nicotinamide

To a solution of 6-(4-(4-(4,4,5,5-tetramethyl-1,3,2-dioxaborolan-2-yl)phenyl)piperidin-1-yl)nicotinamide (Intermediate 317, 3.09 g, 7.59 mmol) in 1,4-dioxane (30 mL) was added 3-(4-iodo-1-oxoisoindolin-2-yl)-1-((2-(trimethylsilyl)ethoxy)methyl)piperidine-2,6-dione (Intermediate 8, 1.90 g, 3.80 mmol) followed by potassium carbonate (1.57 g, 11.4 mmol). The reaction mixture was purged with argon for 15 min then PdCl₂(dppf)·DCM (0.31 g, 0.38 mmol) was added. The reaction mixture was purged with argon for another 10 min and then stirred at 100° C. for 4 h. The mixture was concentrated in vacuo and the crude material was purified by combi-flash column chromatography, by eluting with 3% MeOH in dichloromethane, to afford 6-(4-(4-(2-(2,6-dioxo-1-((2-(trimethylsilyl)ethoxy)methylpiperidin-3-yl)-1-oxoisoindolin-4-yl)phenyl)piperidin-1-yl)nicotinamide (Intermediate 318, 1.20 g, 48%) as a yellow solid. ¹H NMR (400 MHz, DMSO-d₆) δ ppm −0.10 (s, 9H), 0.71-0.82 (m, 2H), 1.54-1.68 (m, 2H), 1.80-1.90 (m, 2H), 1.96-2.04 (m, 1H), 2.36-2.40 (m, 1H), 2.70-2.78 (m, 1H), 2.86-3.04 (m, 4H), 3.42-3.52 (m, 2H), 4.28-4.32 (m, 1H), 4.52-4.63 (m, 3H), 4.95-5.05 (m, 2H), 5.23-5.27 (m, 1H), 6.84-6.86 (m, 1H), 7.05 (br s, 1H), 7.35 (d, J=8.20 Hz, 2H), 7.49 (d, J=8.20 Hz, 2H), 7.57-7.74 (m, 4H), 7.93 (dd, J=9.12, 2.07 Hz, 1H), 8.60 (d, J=2.07 Hz, 1H). Mass spec m/z 654.1 [M+H]⁺.

### Step 5

### Intermediate 319: tert-butyl-6-(6-(4-(4-(2-(2,6-dioxo-1-((2-(trimethylsilyl)ethoxy)methyl)piperidin-3-yl)-1-oxoisoindolin-4-yl)phenyl)piperidin-1-yl)nicotinamido)-2-((R)-1-methylpyrrolidin-2-yl)-1H-pyrrolo[3,2-c]pyridine-1-carboxylate

To a solution of 6-(4-(4-(2-(2,6-dioxo-1-((2-(trimethylsilyl)ethoxy)methyl)piperidin-3-yl)-1-oxoisoindolin-4-yl)phenyl)piperidin-1-yl)nicotinamide (Intermediate 318, 1.10 g, 1.7 mmol) in 1,4-dioxane (18 mL) was added tert-butyl-(R)-6-bromo-2-(1-methylpyrrolidin-2-yl)-1H-pyrrolo[3,2-c]pyridine-1-carboxylate (Intermediate 4, 0.70 g, 1.9 mmol) followed by cesium carbonate (1.5 g, 4.5 mmol). The reaction mixture was purged with argon for 15 min then Pd₂(dba)₃ (0.24 g, 0.25 mmol) and Xantphos (0.30 g, 0.50 mmol) were added. The reaction mixture was purged with argon for another 10 min and then stirred at 90° C. for 3 h. The reaction mixture was filtered through celite bed and the filter pad was washed with ethyl acetate (100 mL), and the filtrate concentrated in vacuo. The obtained solid residue was washed with diethyl ether (2×50 mL) dried in vacuo to afford tert-butyl-6-(6-(4-(4-(2-(2,6-dioxo-1-((2-(trimethylsilyl)ethoxy)methyl)piperidin-3-yl)-1-oxoisoindolin-4-yl)phenyl)piperidin-1-yl)nicotinamido)-2-((R)-1-methylpyrrolidin-2-yl)-1H-pyrrolo[3,2-c]pyridine-1-carboxylate (Intermediate 319, 0.80 g) as a yellow solid, which was used for the next step without further purification. ¹H NMR (400 MHz, DMSO-d₆) δ ppm −0.08 (s, 9H), 0.76-0.84 (m, 2H), 1.06-1.11 (m, 2H), 1.56-1.62 (m, 2H), 1.69 (s, 9H), 1.72-1.80 (m, 2H), 1.86-1.96 (m, 2H), 1.98-2.07 (m, 3H), 2.29-2.33 (m, 2H), 2.35 (s, 3H) 2.38-2.42 (m, 1H), 2.76-2.80 (m, 1H), 3.13-3.25 (m, 1H), 3.35-3.41 (m, 2H), 3.48-3.52 (m, 2H), 4.40-4.44 (m, 2H), 4.58-4.69 (m, 2H), 4.99-5.08 (m, 2H), 5.12-5.16 (m, 1H), 6.74 (s, 1H), 6.93-6.95 (m, 1H), 7.34-7.40 (m, 1H), 7.50-7.56 (m, 2H), 7.62-7.66 (m, 1H), 7.67-7.76 (m, 2H), 8.15-8.21 (m, 2H), 8.57 (s, 1H), 8.83 (s, 1H), 8.92 (s, 1H), 10.50 (br s, 1H). Mass spec m/z 952.8 [M+H]⁺.

### Step 6

### Example 67: 6-(4-(4-(2-(2,6-dioxopiperidin-3-yl)-1-oxoisoindolin-4-yl)phenyl)piperidin-1-yl)-N-(2-((R)-1-methylpyrrolidin-2-yl)-1H-pyrrolo[3,2-c]pyridin-6-yl)nicotinamide

To a solution of tert-butyl-6-(6-(4-(4-(2-(2,6-dioxo-1-((2-(trimethylsilyl)ethoxy)methyl)piperidin-3-yl)-1-oxoisoindolin-4-yl)phenyl)piperidin-1-yl)nicotinamido)-2-((R)-1-methylpyrrolidin-2-yl)-1H-pyrrolo[3,2-c]pyridine-1-carboxylate (Intermediate 319, 0.75 g, 0.78 mmol) in acetonitrile (8.0 mL) was added methanesulfonic acid (0.52 mL, 7.86 mmol) and the reaction mixture was stirred at 60° C. for 2 h. After cooling to room temperature, N,N′-dimethylethylenediamine (0.47 mL, 3.93 mmol) followed by triethylamine (2.20 mL, 15.74 mmol) were added the reaction mixture was stirred at room temperature for 3 h. The reaction mixture was concentrated in vacuo and the obtained residue was diluted with water (50 mL). The resultant precipitate was collected by filtration and dried in vacuo. The crude solid was purified by preparative HPLC (Method B) to afford 6-(4-(4-(2-(2,6-dioxopiperidin-3-yl)-1-oxoisoindolin-4-yl)phenyl)piperidin-1-yl)-N-(2-((R)-1-methylpyrrolidin-2-yl)-1H-pyrrolo[3,2-c]pyridin-6-yl)nicotinamide (Example 67, 0.03 g, 7%) as an off white solid. ¹H NMR (400 MHz, DMSO-d₆) δ ppm 1.60-1.74 (m, 2H), 1.80-2.05 (m, 6H), 2.22 (s, 3H), 2.42-2.46 (m, 2H), 2.56-2.58 (m, 2H), 2.83-3.08 (m, 5H), 3.13-3.25 (m, 1H), 4.40-4.44 (m, 1H), 4.58-4.69 (m, 3H), 5.12-5.16 (m, 1H), 6.45 (s, 1H), 6.93-6.95 (m, 1H), 7.40 (d, J=8.40 Hz, 2H), 7.54 (d, J=8.40 Hz, 2H), 7.60-7.66 (m, 1H), 7.67-7.76 (m, 2H), 8.15-8.21 (m, 2H), 8.52 (s, 1H), 8.81-8.82 (m, 1H), 10.32 (br s, 1H), 10.97 (s, 1H), 11.41 (br s, 1H). Mass spec m/z 723.0 [M+H]⁺.

### Example 68 was Synthesised Following Scheme 66

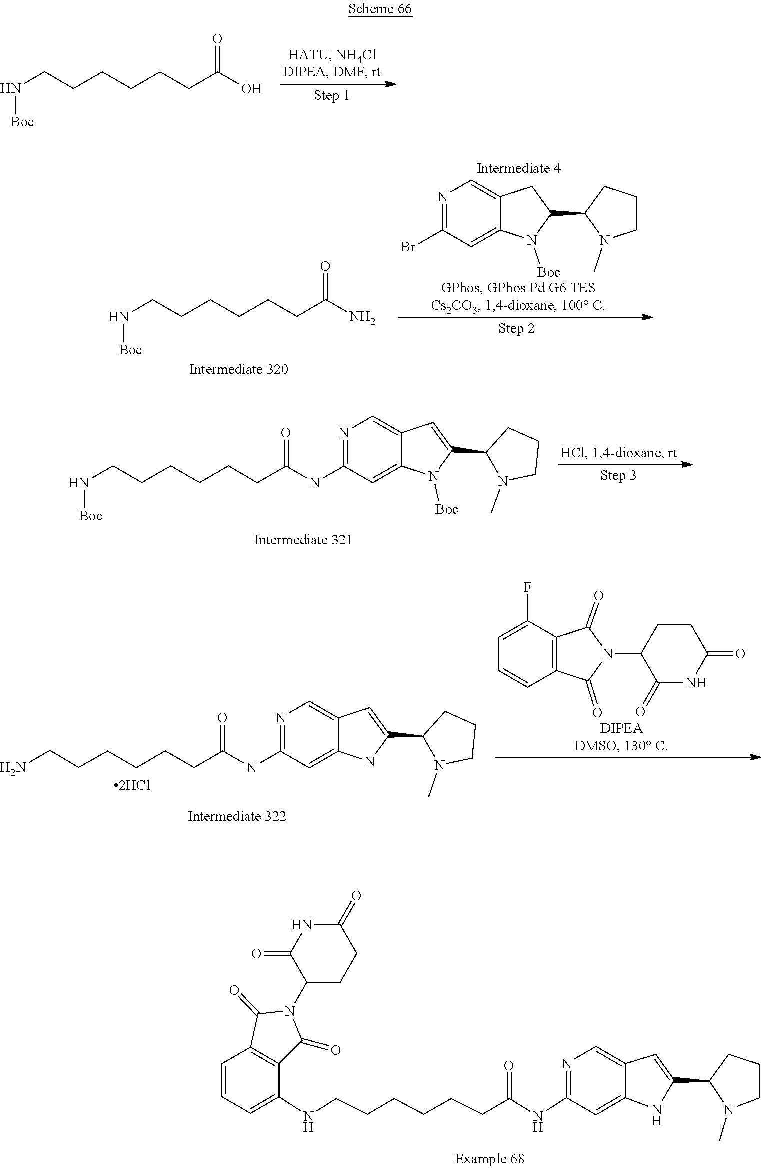

### Step 1

### Intermediate 320: tert-butyl N-(6-carbamoylhexyl)carbamate

To a solution of 7-[(tert-butoxycarbonyl)amino]heptanoic acid (CAS No: 60142-89-4, 500 mg, 2.04 mmol) in DMF (5 mL) were added DIPEA (790 mg, 6.12 mmol), HATU (1.16 g, 3.06 mmol) and NH₄Cl (545 mg, 10.2 mmol). The reaction mixture was stirred for 2 h at rt. The reaction mixture was diluted with water and extracted three times with EtOAc. The combined organic layers were washed with brine, dried over anhydrous Na₂SO₄, filtered and concentrated under reduced pressure. The crude material was purified by flash chromatography on silica gel using petroleum ether/EtOAc (9/1) as eluent to afford tert-butyl N-(6-carbamoylhexyl)carbamate (Intermediate 320, 363 mg, 73%) as a white solid. Mass spec: m/z: 315 [M+H]⁺.

### Step 2

### Intermediate 321: tert-butyl 6-{7-[(tert-butoxycarbonyl)amino]heptanamido}-2-[(2R)-1-methylpyrrolidin-2-yl]pyrrolo[3,2-c]pyridine-1-carboxylate

To a solution of tert-butyl N-(6-carbamoylhexyl)carbamate (Intermediate 320, 200 mg, 0.737 mmol), tert-butyl 6-bromo-2-[(rel-2R)-1-methylpyrrolidin-2-yl]pyrrolo[3,2-c]pyridine-1-carboxylate (Intermediate 4, 364 mg, 0.958 mmol) and Cs₂CO₃ (720 mg, 2.21 mmol in 1,4-dioxane (4 mL) and) were added GPhos (80 mg, 0.147 mmol) and GPhos Pd G6 TES (70 mg, 0.0740 mmol). The reaction mixture was stirred at 100° C. overnight under nitrogen atmosphere, then concentrated under reduced pressure. The residue was chromatographed by reverse-phase flash chromatography (C18) using MeCN/H₂O (36/64) as eluent to afford tert-butyl 6-{7-[(tert-butoxycarbonyl)amino]heptanamido}-2-[(2R)-1-methylpyrrolidin-2-yl]pyrrolo[3,2-c]pyridine-1-carboxylate (Intermediate 321, 310 mg, 69%) as a yellow solid. Mass spec: m/z: 544 [M+H]⁺.

### Step 3

### Intermediate 322: 7-amino-N-{2-[(2R)-1-methylpyrrolidin-2-yl]-1H-pyrrolo[3,2-c]pyridin-6-yl}heptanamide dihydrochloride

To a solution of tert-butyl 6-{7-[(tert-butoxycarbonyl)amino]heptanamido}-2-[(rel-2R)-1-methylpyrrolidin-2-yl]pyrrolo[3,2-c]pyridine-1-carboxylate (Intermediate 321, 310 mg, 0.507 mmol) in 1,4-dioxane (10 mL) was added a solution of HCl (2 mL, 12N in H₂O). The reaction mixture was stirred at rt overnight, then concentrated under reduced pressure to afford 7-amino-N-{2-[(2R)-1-methylpyrrolidin-2-yl]-1H-pyrrolo[3,2-c]pyridin-6-yl}heptanamide dihydrochloride (Intermediate 322, 210 mg, 49%) as a yellow solid which was used in Step 4 without further purification. Mass spec: m/z: 344 [M+H]⁺.

### Step 4

### Example 68: 7-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}heptanamide

A solution of 7-amino-N-{2-[(2R)-1-methylpyrrolidin-2-yl]-1H-pyrrolo[3,2-c]pyridin-6-yl}heptanamide dihydrochloride (Intermediate 322, 200 mg, 0.262 mmol) in DMSO (4 mL) were added 2-(2,6-dioxopiperidin-3-yl)-4-fluoroisoindole-1,3-dione (CAS: 835616-60-9, 145 mg, 0.524 mmol) and DPIEA (339 mg, 2.62 mmol). The reaction mixture was stirred at 130° C. for 2 h, then quenched with water and concentrated under reduced pressure. The residue was purified by preparative HPLC (Column: Xselect CSH OBD, 30×150 mm, 5 μm; Eluent: Water (+0.1% formic acid)/MeCN from 98/2 to 70/30; Flow rate: 60 mL/min mL/min) to afford 7-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}heptanamide (Example 68, 46.8 mg, 27%) as a yellow solid. Mass spec: m/z: 600 [M+H]+; ¹H NMR (400 MHz, DMSO-d₆) δ ppm 11.25 (s, 1H), 11.10 (s, 1H), 10.10 (s, 1H), 8.40 (s, 1H), 8.15 (s, 1H), 8.07 (s, 1H), 7.54-7.58 (m, 1H), 7.07-7.10 (m, 1H), 7.00-7.02 (s, 1H), 6.52-6.55 (m, 1H), 6.33 (s, 1H), 5.02-5.07 (m, 1H), 3.28-3.31 (m, 3H), 3.12-3.26 (m, 1H), 2.82-2.94 (m, 1H), 2.50-2.60 (m, 2H), 2.34-2.38 (m, 2H), 2.24-2.26 (m, 1H), 2.11-2.14 (m, 4H), 2.01-2.04 (m, 1H), 1.79-1.81 (m, 3H), 1.43-1.65 (m, 4H), 1.31-1.42 (m, 4H).

### Example 69 was Synthesised Following Scheme 67

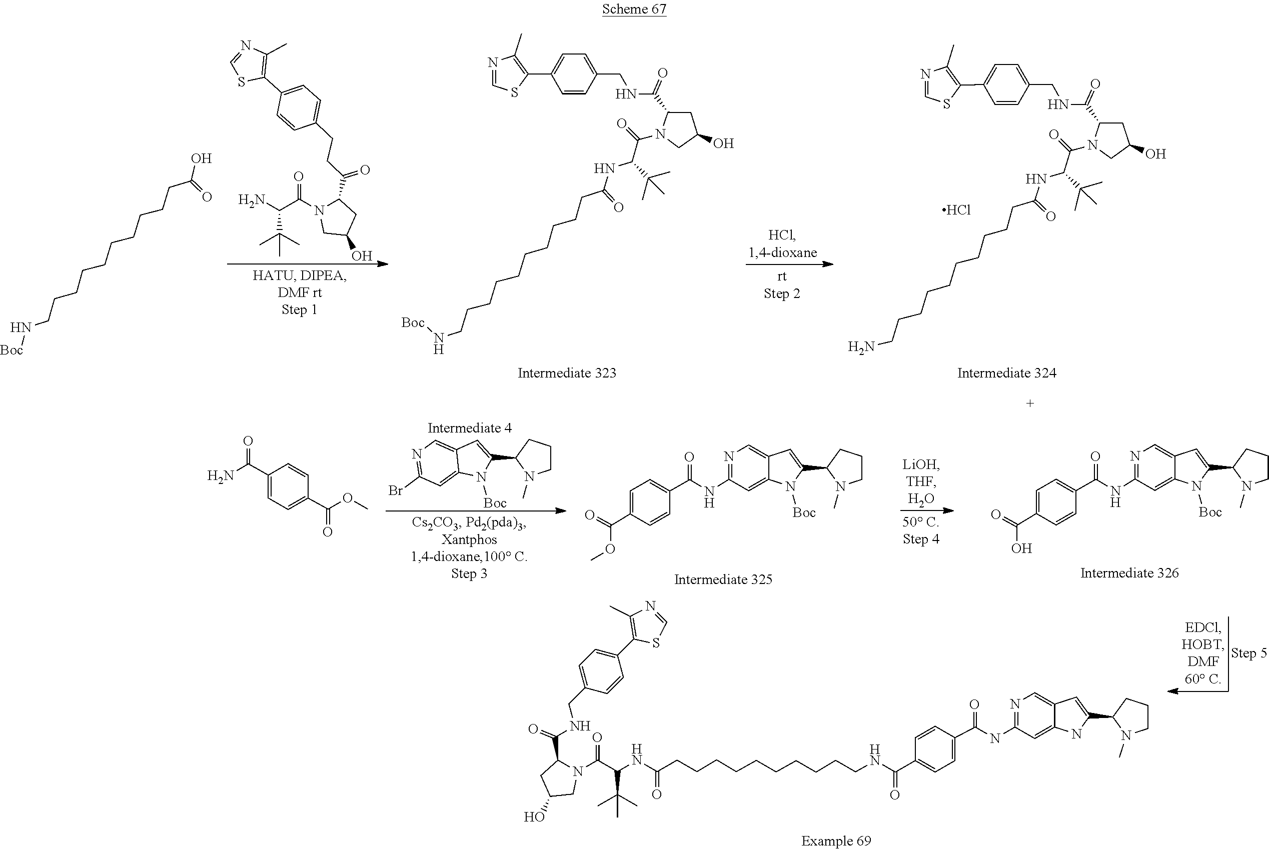

### Step 1

### Intermediate 323: tert-butyl N-(10-{[(2S)-1-[(2S,4R)-4-hydroxy-2-({[4-(4-methyl-1,3-thiazol-5-yl)phenyl]methyl}carbamoyl)pyrrolidin-1-yl]-3,3-dimethyl-1-oxobutan-2-yl]carbamoyl}decyl)carbamate

To a solution of 11-[(tert-butoxycarbonyl)amino]undecanoic acid (CAS No: 10436-25-6, 5 g, 16.5 mmol) in DMF (100 mL) was added (2S,4R)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide hydrochloride (CAS No: 1448189-80-7, 7.14 g, 16.5 mmol), DIPEA (8.58 g, 66.3 mmol) and HATU (8.20 g, 21.5 mmol) under a nitrogen atmoshphere. The reaction mixture was stirred at rt for 4 h. Water (200 mL) was added and the resulting mixture was extracted with EtOAc (3×500 mL) and the organic layers were combined, washed with brine (200 mL), dried over anhydrous sodium sulfate, filtered and concentrated under reduced pressure. The residue was chromatographed on a silica gel column, eluting with DCM/MeOH (20/1), to afford tert-butyl N-(10-{[(2S)-1-[(2S,4R)-4-hydroxy-2-({[4-(4-methyl-1,3-thiazol-5-yl)phenyl]methyl}carbamoyl)pyrrolidin-1-yl]-3,3-dimethyl-1-oxobutan-2-yl]carbamoyl}decyl)carbamate (Intermediate 323, 8 g, 67.5%) as a yellow solid. Mass spec: m/z: 714.7 [M+H]⁺.

### Step 2

### Intermediate 324: (2S,4R)-1-[(2S)-2-(11-aminoundecanamido)-3,3-dimethylbutanoyl]-4-hydroxy-N-{[4-(4-methyl-1,3-thiazol-5-yl)phenyl]methyl}pyrrolidine-2-carboxamide hydrochloride

To a solution of tert-butyl N-(10-{[(2S)-1-[(2S,4R)-4-hydroxy-2-({[4-(4-methyl-1,3-thiazol-5-yl)phenyl]methyl}carbamoyl)pyrrolidin-1-yl]-3,3-dimethyl-1-oxobutan-2-yl]carbamoyl}decyl)carbamate (Intermediate 323, 700 mg, 0.980 mmol) in 1,4-dioxane (3 mL) was added HCl (3 mL, 4M in 1,4-dioxane) and the reaction mixture was stirred at room temperature for 1 h. The reaction was then concentrated under reduced pressure to afford (2S,4R)-1-[(2S)-2-(11-aminoundecanamido)-3,3-dimethylbutanoyl]-4-hydroxy-N-{[4-(4-methyl-1,3-thiazol-5-yl)phenyl]methyl}pyrrolidine-2-carboxamide hydrochloride (Intermediate 324, 600 mg) as a yellow solid, which was used in the next step without further purification. Mass spec: m/z: 614.4 [M+H]⁺.

### Step 3

### Intermediate 325: tert-butyl 6-[4-(methoxycarbonyl)benzamido]-2-[(2R)-1-methylpyrrolidin-2-yl]pyrrolo[3,2-c]pyridine-1-carboxylate

To a solution of tert-butyl 6-bromo-2-[(2R)-1-methylpyrrolidin-2-yl]pyrrolo[3,2-c]pyridine-1-carboxylate (Intermediate 4, 500 mg, 1.31 mmol) in 1,4-dioxane (10 mL) was added methyl 4-carbamoylbenzoate (CAS No: 6757-31-9, 235 mg, 1.31 mmol), Cs₂CO₃ (1.29 g, 3.94 mmol), Xantphos (152 mg, 0.263 mmoL) and Pd₂(dba)₃ (120 mg, 0.131 mmol) and the reaction was stirred at 100° C. overnight under N₂. The mixture was quenched with water (100 mL) and extracted with EtOAc (3×100 mL). The combined organic layers were washed with brine, dried over anhydrous Na₂SO₄, filtered and concentrated under reduced pressure. The residue was chromatographed on a silica gel column, eluting with DCM/MeOH (95/5), to afford tert-butyl6-[4-(methoxycarbonyl)benzamido]-2-[(2R)-1-methylpyrrolidin-2-yl]pyrrolo[3,2-c]pyridine-1-carboxylate (Intermediate 325, 300 mg, 41%) as a yellow solid. Mass spec: m/z: 479.3 [M+H]⁺.

### Step 4

### Intermediate 326: 4-({2-[(2R)-1-methylpyrrolidin-2-yl]-1H-pyrrolo[3,2-c]pyridin-6-yl}carbamoyl)benzoic acid

To a solution of tert-butyl 6-[4-(methoxycarbonyl)benzamido]-2-[(2R)-1-methylpyrrolidin-2-yl]pyrrolo[3,2-c]pyridine-1-carboxylate (Intermediate 325, 260 mg, 0.543 mmol) in THF (5 mL) was added H₂O (5 mL) and LiOH (104 mg, 4.34 mmol) and the reaction was stirred at 50° C. for 2 h. Water (50 mL) was added the mixture was adjusted to pH 4-6 by dropwise addition of concentrated HCl. The resulting solution was extracted with EtOAc (3×100 mL) and the combined organic phases were dried over anhydrous sodium sulfate and concentrated under vacuum to afford 4-({2-[(2R)-1-methylpyrrolidin-2-yl]-1H-pyrrolo[3,2-c]pyridin-6-yl}carbamoyl)benzoic acid (Intermediate 326, 190 mg, 86%) as a yellow solid, which was used in the next step without further purification. Mass spec: m/z: 365.3 [M+H]⁺.

### Step 5

### Example 69: (2S,4R)-1-[(2S)-3,3-dimethyl-2-(11-{[4-({2-[(2R)-1-methylpyrrolidin-2-yl]-1H-pyrrolo[3,2-c]pyridin-6-yl}carbamoyl)phenyl]formamido}undecanamido)butanoyl]-4-hydroxy-N-{[4-(4-methyl-1,3-thiazol-5-yl)phenyl]methyl}pyrrolidine-2-carboxamide

To a solution of 4-({2-[(2R)-1-methylpyrrolidin-2-yl]-1H-pyrrolo[3,2-c]pyridin-6-yl}carbamoyl)benzoic acid (Intermediate 326, 50.0 mg, 0.134 mmol) in DMF (1.5 mL) was added (2S,4R)-1-[(2S)-2-(11-aminoundecanamido)-3,3-dimethylbutanoyl]-4-hydroxy-N-{[4-(4-methyl-1,3-thiazol-5-yl)phenyl]methyl}pyrrolidine-2-carboxamide hydrochloride (Intermediate 324, 84.2 mg, 0.137 mmol), EDCI (31.5 mg, 0.164 mmol) and HOBT (24.1 mg, 0.178 mmol) and the resulting mixture was stirred at 60° C. overnight. The crude product was purified by preparative HPLC (Column: XBridge Prep Shield RP C18 Column, 19*250 mm, 5 μm; Mobile Phase A: Water (+0.1% formic acid), Mobile Phase B: MeOH; Flow rate: 25 mL/min; Gradient: 38% B to 68% B in 10 min) to afford (2S,4R)-1-[(2S)-3,3-dimethyl-2-(11-{[4-({2-[(2R)-1-methylpyrrolidin-2-yl]-1H-pyrrolo[3,2-c]pyridin-6-yl}carbamoyl)phenyl]formamido}undecanamido)butanoyl]-4-hydroxy-N-{[4-(4-methyl-1,3-thiazol-5-yl)phenyl]methyl}pyrrolidine-2-carboxamide (Example 69, 14.2 mg, 10.7%) as a white solid. ¹H NMR (400 MHz, MeOH-d4) δ ppm 8.91 (s, 1H), 8.55 (s, 1H), 8.21 (s, 1H), 8.08 (d, J=8.4 Hz, 2H), 7.97 (d, J=8.8 Hz, 2H), 7.40-7.48 (m, 4H), 6.56 (s, 1H), 4.65 (s, 1H), 4.59-4.64 (m, 2H), 4.50-4.56 (m, 1H), 4.34-4.38 (m, 1H), 3.88-3.90 (m, 1H), 3.82-3.83 (m, 1H), 3.41-3.49 (m, 1H), 3.33-3.39 (m, 2H), 2.48-2.49 (m, 4H), 2.34-2.36 (m, 3H), 2.26-2.29 (m, 4H), 2.06-2.10 (m, 3H), 1.90-1.93 (m, 1H), 1.61-1.67 (m, 4H), 1.34-1.39 (m, 13H), 1.04 (s, 9H). Mass spec: m/z: 962.65.

### Example 70 was Synthesised Following Scheme 68

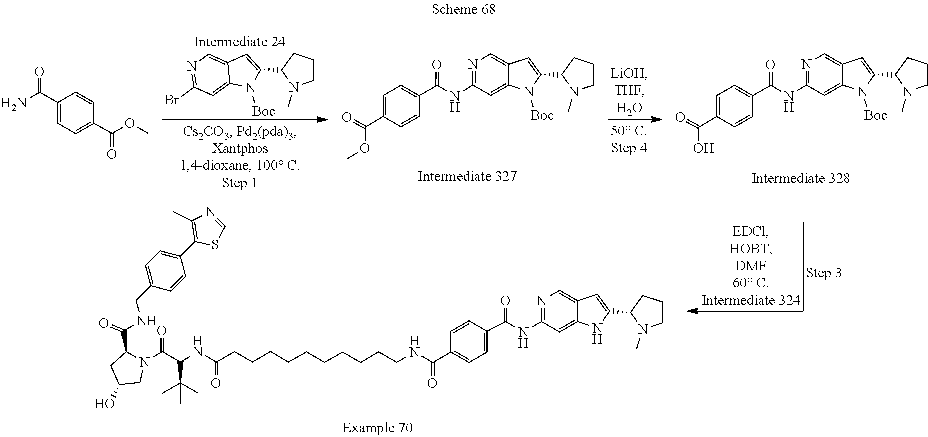

### Step 1

### Intermediate 327: tert-butyl 6-[4-(methoxycarbonyl)benzamido]-2-[(2S)-1-methylpyrrolidin-2-yl]pyrrolo[3,2-c]pyridine-1-carboxylate

Intermediate 327 was synthesised following an analogous procedure used for Intermediate 325 shown in Step 3 of Scheme 67, using methyl 4-carbamoylbenzoate (CAS No: 6757-31-9) as the amide and tert-butyl 6-bromo-2-[(2S)-1-methylpyrrolidin-2-yl]pyrrolo[3,2-c]pyridine-1-carboxylate (Intermediate 24) to afford tert-butyl 6-[4-(methoxycarbonyl)benzamido]-2-[(2S)-1-methylpyrrolidin-2-yl]pyrrolo[3,2-c]pyridine-1-carboxylate (Intermediate 327). Mass spec: m/z: 479.3 [M+H]⁺.

### Step 2

### Intermediate 328: 4-({2-[(2S)-1-methylpyrrolidin-2-yl]-1H-pyrrolo[3,2-c]pyridin-6-yl}carbamoyl)benzoic acid

Intermediate 328 was synthesised following an analogous procedure used for Intermediate 325 shown in Step 4 of Scheme 67, using tert-butyl 6-[4-(methoxycarbonyl)benzamido]-2-[(2S)-1-methylpyrrolidin-2-yl]pyrrolo[3,2-c]pyridine-1-carboxylate (Intermediate 327) as the ester to afford 4-({2-[(2S)-1-methylpyrrolidin-2-yl]-1H-pyrrolo[3,2-c]pyridin-6-yl}carbamoyl)benzoic acid (Intermediate 328). Mass spec: 365.3 [M+H]⁺.

### Step 3

### Example 70: (2S,4R)-1-[(2S)-3,3-dimethyl-2-(11-{[4-({2-[(2S)-1-methylpyrrolidin-2-yl]-1H-pyrrolo[3,2-c]pyridin-6-yl}carbamoyl)phenyl]formamido}undecanamido)butanoyl]-4-hydroxy-N-{[4-(4-methyl-1,3-thiazol-5-yl)phenyl]methyl}pyrrolidine-2-carboxamide

Example 70 was synthesised following an analogous procedure used for Example 69, shown in Step 5 of Scheme 67, using 4-({2-[(2S)-1-methylpyrrolidin-2-yl]-1H-pyrrolo[3,2-c]pyridin-6-yl}carbamoyl)benzoic acid (Intermediate 328) as the acid and (2S,4R)-1-[(2S)-2-(11-aminoundecanamido)-3,3-dimethylbutanoyl]-4-hydroxy-N-{[4-(4-methyl-1,3-thiazol-5-yl)phenyl]methyl}pyrrolidine-2-carboxamide hydrochloride (Intermediate 324) as the amine, to afford (2S,4R)-1-[(2S)-3,3-dimethyl-2-(11-{[4-({2-[(2S)-1-methylpyrrolidin-2-yl]-1H-pyrrolo[3,2-c]pyridin-6-yl}carbamoyl)phenyl]formamido}undecanamido)butanoyl]-4-hydroxy-N-{[4-(4-methyl-1,3-thiazol-5-yl)phenyl]methyl}pyrrolidine-2-carboxamide (Example 70). ¹H NMR (400 MHz, DMSO-d6) δ ppm 11.41 (s, 1H), 10.63 (s, 1H), 8.98 (s, 1H), 8.51-8.59 (m, 3H), 8.21 (s, 1H), 8.11 (d, J=8.4 Hz, 2H), 7.94 (d, J=8.4 Hz, 2H), 7.83 (d, J=9.2 Hz, 1H), 7.37-7.43 (m, 4H), 6.40 (s, 1H), 4.54 (d, J=9.6 Hz, 1H), 4.43-4.50 (m, 2H), 4.35-4.37 (m, 1H), 4.23-4.25 (m, 1H), 3.67-3.70 (m, 1H), 3.31-3.35 (m, 1H), 3.24-3.28 (m, 2H), 3.15-3.22 (m, 1H), 2.44 (s, 3H), 2.24-2.28 (m, 2H), 2.17 (s, 3H), 2.11-2.14 (m, 3H), 1.88-1.92 (m, 4H), 1.49-1.53 (m, 4H), 1.25-1.29 (m, 14H), 0.93 (s, 9H). Mass spec: m/z: 960.75 [M+H]⁺.

### Example 71 was Synthesized Following Scheme 69

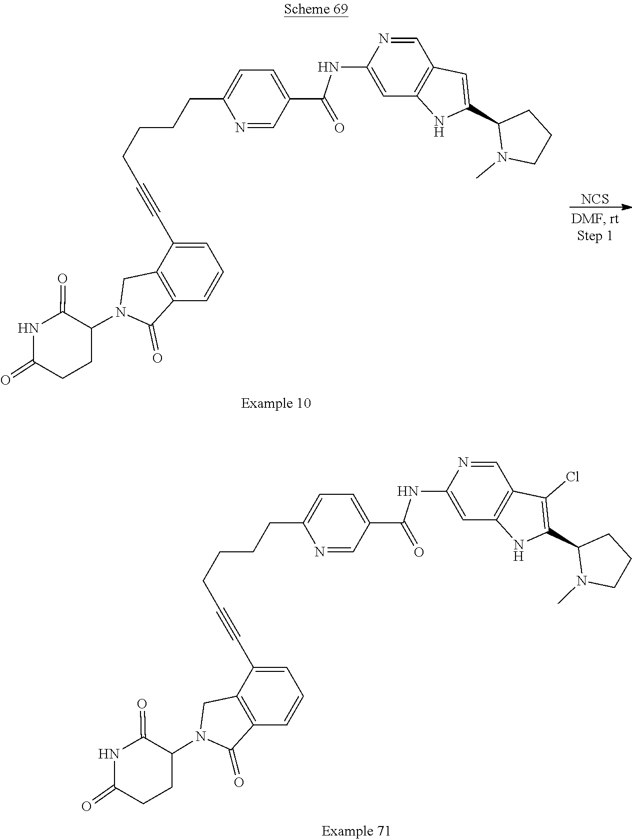 [SMILES: CN1CCC[C@@H]1c1cc2cnc(NC(=O)c3ccc(CCCCC#Cc4cccc5c4CN(C4CCC(=O)NC4=O)C5=O)nc3)cc2[nH]1 ; "NCS" ; CN1CCC[C@@H]1c1[nH]c2cc(NC(=O)c3ccc(CCCCC#Cc4cccc5c4CN(C4CCC(=O)NC4=O)C5=O)nc3)ncc2c1Cl]

### Step 1

### Example 71: N-(3-chloro-2-((R)-1-methylpyrrolidin-2-yl)-1H-pyrrolo[3,2-c]pyridin-6-yl)-6-(6-(2-(2,6-dioxopiperidin-3-yl)-1-oxoisoindolin-4-yl) hex-5-yn-1-yl) nicotinamide

To a solution of 6-(6-(2-(2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)nicotinamide (Example 10, 0.050 g, 0.077 mmol) in dimethylformamide (1 mL) was added N-chlorosuccinimide (0.016 g, 0.11 mmol) and the reaction mixture was stirred at room temperature for 30 h. The reaction mixture was quenched with water (20 mL) and extracted with ethyl acetate (2×20 mL). The combined organic layers were washed with saturated brine solution (20 mL), dried over anhydrous Na₂SO₄ and concentrated in vacuo. The crude material was purified by preparative HPLC (Method A) to afford N-(3-chloro-2-((R)-1-methylpyrrolidin-2-yl)-1H-pyrrolo[3,2-c]pyridin-6-yl)-6-(6-(2-(2,6-dioxopiperidin-3-yl)-1-oxoisoindolin-4-yl)hex-5-yn-1-yl) nicotinamide (Example 71, 0.006 g, 11% yield) as a white solid. ¹H NMR (400 MHz, DMSO-d₆) δ ppm 1.59-1.68 (m, 2H), 1.81-1.92 (m, 4H), 1.94-2.06 (m, 2H), 2.16 (s, 3H), 2.18-2.21 (m, 1H), 2.31-2.34 (m, 1H), 2.40-2.45 (m, 1H), 2.56-2.62 (m, 3H), 2.84-2.92 (m, 3H), 3.12-3.21 (m, 1H), 3.50-3.54 (m, 1H), 4.29-4.35 (m, 1H), 4.42-4.49 (m, 1H), 5.11-5.16 (m, 1H), 7.41 (d, J=8.0 Hz, 1H), 7.50 (t, J=7.60 Hz, 1H), 7.64 (d, J=7.60 Hz, 1H), 7.70 (d, J=7.60 Hz, 1H), 8.26 (s, 1H), 8.29 (m, 1H), 8.51 (s, 1H), 9.08 (s, 1H), 10.83 (s, 1H), 10.99 (s, 1H), 11.71 (s, 1H). Mass spec m/z 678.0 [M+H]⁺.

### Example 72 was Synthesized Following Scheme 70

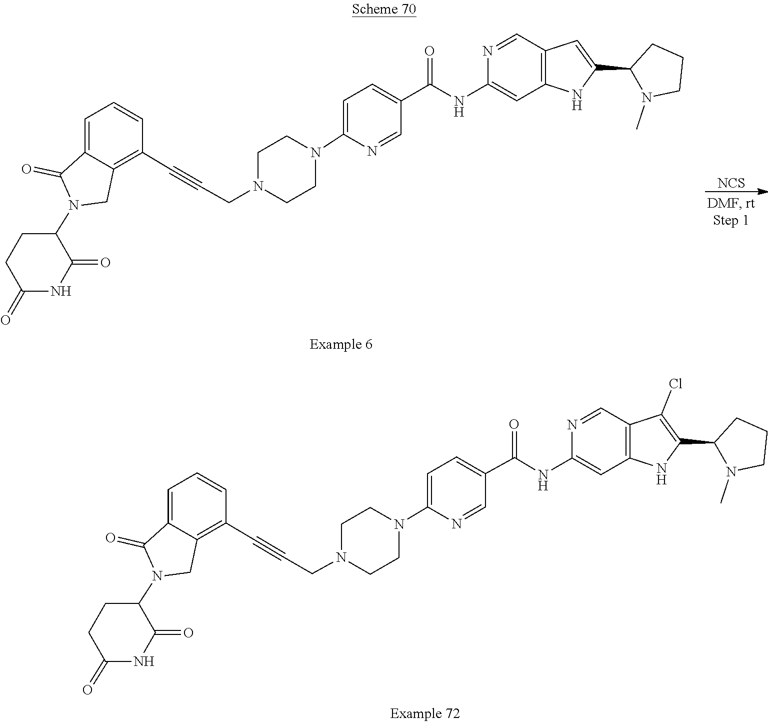 [SMILES: CN1CCC[C@@H]1c1cc2cnc(NC(=O)c3ccc(N4CCN(CC#Cc5cccc6c5CN(C5CCC(=O)NC5=O)C6=O)CC4)nc3)cc2[nH]1 ; "NCS" ; CN1CCC[C@@H]1c1[nH]c2cc(NC(=O)c3ccc(N4CCN(CC#Cc5cccc6c5CN(C5CCC(=O)NC5=O)C6=O)CC4)nc3)ncc2c1Cl]

### Step 1

### Example 72: N-(3-chloro-2-((R)-1-methylpyrrolidin-2-yl)-1H-pyrrolo[3,2-c]pyridin-6-yl)-6-(4-(3-(2-(2,6-dioxopiperidin-3-yl)-1-oxoisoindolin-4-yl)prop-2-yn-1-yl)piperazin-1-yl)nicotinamide

To a solution of 6-(4-(3-(2-(2,6-dioxopiperidin-3-yl)-1-oxoisoindolin-4-yl)prop-2-yn-1-yl)piperazin-1-yl)-N-(2-((R)-1-methylpyrrolidin-2-yl)-1H-pyrrolo[3,2-c]pyridin-6-yl)nicotinamide (Example 6, 0.09 g, 0.13 mmol) in dimethylformamide (0.5 mL) was added N-chlorosuccinimide (0.027 g, 0.19 mmol) and the reaction mixture was stirred at room temperature for 30 h. The reaction mixture was quenched with water (20 mL), the resultant precipitate was collected by filtration and dried in vacuo. The crude material was purified by preparative HPLC (Method A) to afford N-(3-chloro-2-((R)-1-methylpyrrolidin-2-yl)-1H-pyrrolo[3,2-c]pyridin-6-yl)-6-(4-(3-(2-(2,6-dioxopiperidin-3-yl)-1-oxoisoindolin-4-yl)prop-2-yn-1-yl)piperazin-1-yl)nicotinamide (Example 72, 0.009 g, 9% yield) as an off-white solid. ¹H NMR (400 MHz, DMSO-d₆) δ ppm 1.80-1.90 (m, 2H), 1.94-2.05 (m, 2H), 2.16 (s, 3H), 2.18-2.21 (m, 1H), 2.29-2.35 (m, 1H), 2.40-2.46 (m, 1H), 2.55-2.61 (m, 1H), 2.62-2.69 (m, 4H), 2.85-2.95 (m, 1H), 3.15-3.20 (m, 1H), 3.48-3.54 (m, 1H), 3.66-3.73 (m, 6H), 4.29-4.37 (m, 1H), 4.43-4.50 (m, 1H), 5.10-5.14 (m, 1H), 6.89 (d, J=9.20 Hz, 1H), 7.54 (t, J=7.60 Hz, 1H), 7.68-7.76 (m, 2H), 8.16 (dd, J=2.80, 9.20 Hz, 1H), 8.23 (s, 1H), 8.48 (s, 1H), 8.80 (s, 1H), 10.43 (s, 1H), 10.98 (s, 1H), 11.65 (br s, 1H). Mass spec m/z 719.80 [M+H]⁺.

### Example 73 was Synthesized Following Scheme 71

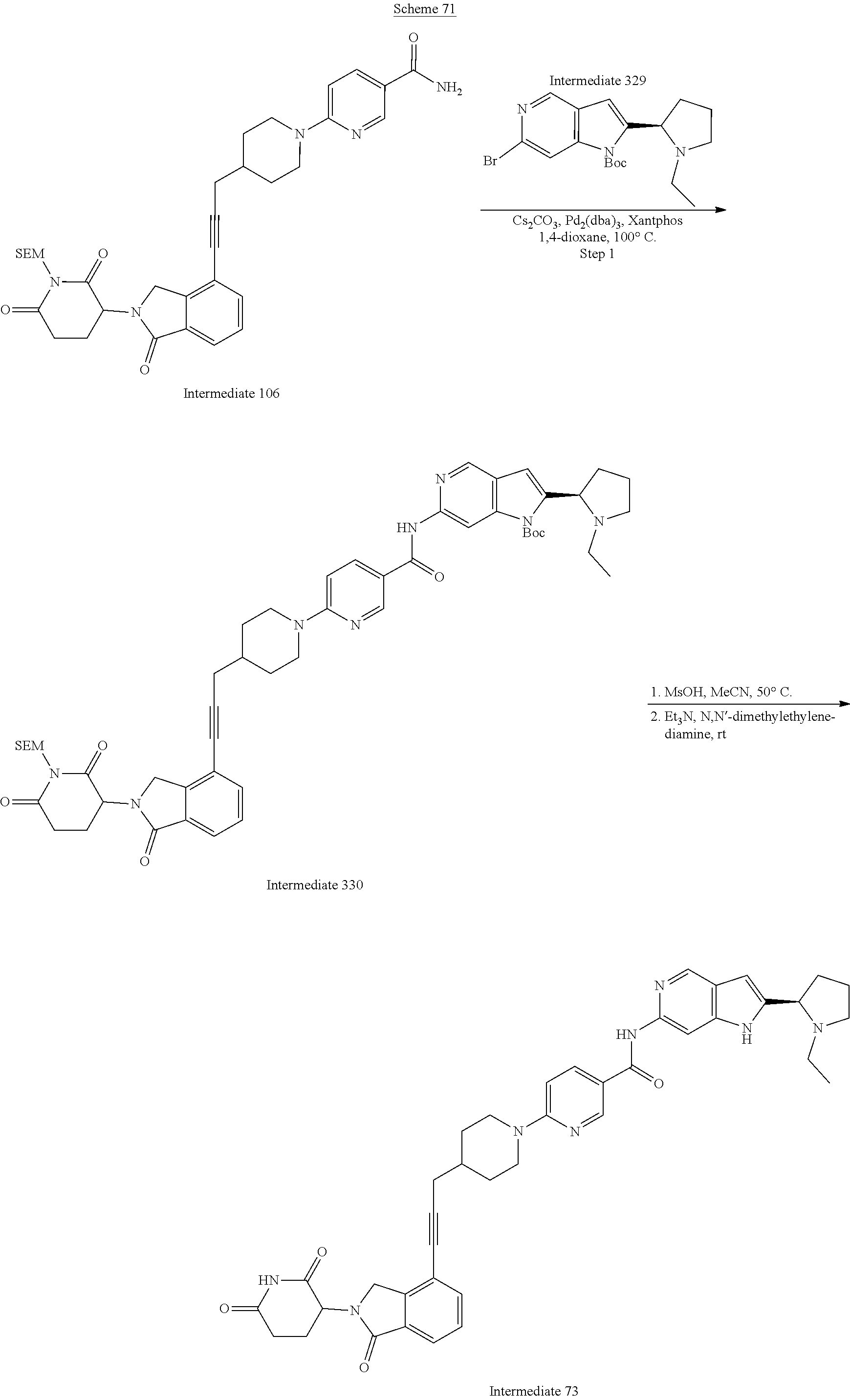 [SMILES: C[Si](C)(C)CCOCN1C(=O)CCC(N2Cc3c(C#CCC4CCN(c5ccc(C(N)=O)cn5)CC4)cccc3C2=O)C1=O ; CCN1CCC[C@@H]1c1cc2cnc(Br)cc2n1C(=O)OC(C)(C)C ; CCN1CCC[C@@H]1c1cc2cnc(NC(=O)c3ccc(N4CCC(CC#Cc5cccc6c5CN(C5CCC(=O)N(COCC[Si](C)(C)C)C5=O)C6=O)CC4)nc3)cc2n1C(=O)OC(C)(C)C ; CCN1CCC[C@@H]1c1cc2cnc(NC(=O)c3ccc(N4CCC(CC#Cc5cccc6c5CN(C5CCC(=O)NC5=O)C6=O)CC4)nc3)cc2[nH]1]

### Step 1

### Intermediate 330: tert-butyl 6-(6-(4-(3-(2-(2,6-dioxo-1-((2-(trimethylsilyl)ethoxy)methyl)piperidin-3-yl)-1-oxoisoindolin-4-yl)prop-2-yn-1-yl)piperidin-1-yl)nicotinamido)-2-((R)-1-ethylpyrrolidin-2-yl)-1H-pyrrolo[3,2-c]pyridine-1-carboxylate

To a solution of 6-(4-(3-(2-(2,6-dioxo-1-((2-(trimethylsilyl)ethoxy)methyl)piperidin-3-yl)-1-oxoisoindolin-4-yl)prop-2-yn-1-yl)piperidin-1-yl)nicotinamide (Intermediate 106, 0.37 g, 0.61 mmol) in 1,4-dioxane (10 mL) was added tert-butyl (R)-6-bromo-2-(1-ethylpyrrolidin-2-yl)-1H-pyrrolo[3,2-c]pyridine-1-carboxylate (Intermediate 329, 0.30 g, 0.76 mmol) followed by cesium carbonate (0.74 g, 2.28 mmol) at room temperature. The reaction mixture was purged with argon for 15 min then Pd₂(dba)₃ (0.11 g, 0.11 mmol) and Xantphos (0.13 g, 0.23 mmol) were added. The reaction mixture was further purged with argon for another 10 min and then stirred at 100° C. for 2 h. The reaction mixture was concentrated in vacuo to obtain crude compound. The crude material was purified by combi-flash chromatography by eluting with 90% ethyl acetate in heptane to afford tert-butyl 6-(6-(4-(3-(2-(2,6-dioxo-1-((2-(trimethylsilyl)ethoxy)methyl)piperidin-3-yl)-1-oxoisoindolin-4-yl)prop-2-yn-1-yl)piperidin-1-yl)nicotinamido)-2-((R)-1-ethylpyrrolidin-2-yl)-1H-pyrrolo[3,2-c]pyridine-1-carboxylate (Intermediate 330, 0.32 g, 45%) as a brown solid. ¹H NMR (400 MHz, DMSO-d₆) δ ppm −0.04 (s, 9H), 0.77-0.87 (m, 2H), 1.24 (t, J=6.80 Hz, 3H), 1.73 (s, 9H), 1.84-1.97 (m, 3H), 2.01-2.22 (m, 5H), 2.38-2.43 (m, 1H), 2.58-2.65 (m, 1H), 2.77-2.82 (m, 1H), 2.93-3.01 (m, 2H), 3.28-3.36 (m, 2H), 3.45-3.59 (m, 8H), 4.25-4.30 (m, 1H), 4.42-4.55 (m, 3H), 5.01-5.08 (m, 2H), 5.21-5.31 (m, 2H), 6.92-6.94 (m, 1H), 7.50-7.57 (m, 2H), 7.65 (d, J=7.60 Hz, 1H), 7.72 (d, J=7.60 Hz, 1H), 8.13-8.20 (m, 1H), 8.74 (s, 1H), 8.77-8.81 (m, 1H), 8.94 (s, 1H), 10.72 (br s, 1H). Mass spec: m/z: 929.7 [M+H]⁺.

### Step 2

### Example 73: 6-(4-(3-(2-(2,6-dioxopiperidin-3-yl)-1-oxoisoindolin-4-yl)prop-2-yn-1-yl)piperidin-1-yl)-N-(2-((R)-1-ethylpyrrolidin-2-yl)-1H-pyrrolo[3,2-c]pyridin-6-yl)nicotinamide

To a solution of tert-butyl 6-(6-(4-(3-(2-(2,6-dioxo-1-((2-(trimethylsilyl)ethoxy)methyl)piperidin-3-yl)-1-oxoisoindolin-4-yl)prop-2-yn-1-yl)piperidin-1-yl)nicotinamido)-2-((R)-1-ethylpyrrolidin-2-yl)-1H-pyrrolo[3,2-c]pyridine-1-carboxylate (Intermediate 330, 0.28 g, 0.30 mmol) in acetonitrile (10 mL) was added methanesulfonic acid (0.29 mL, 4.52 mmol) at room temperature and the reaction mixture was stirred at 50° C. for 2 h. N,N′-dimethylethylenediamine (0.21 mL, 1.81 mmol) followed by triethylamine (0.61 g, 6.03 mmol) and the reaction stirred at room temperature for 3 h. The reaction mixture was diluted with water (100 mL), resulting in a precipitate which was filtered and dried to obtain crude compound. The crude material was purified by preparative HPLC (Method A) to afford 6-(4-(3-(2-(2,6-dioxopiperidin-3-yl)-1-oxoisoindolin-4-yl)prop-2-yn-1-yl)piperidin-1-yl)-N-(2-((R)-1-ethylpyrrolidin-2-yl)-1H-pyrrolo[3,2-c]pyridin-6-yl)nicotinamide (Example 73, 0.14 g, 66%) as an off white solid. ¹H NMR (400 MHz, DMSO-d₆) δ ppm 0.98 (t, J=7.19 Hz, 3H), 1.24-1.36 (m, 2H), 1.78-1.94 (m, 6H), 1.97-2.05 (m, 1H), 2.10-2.24 (m, 3H), 2.39-2.45 (m, 1H), 2.54-2.64 (m, 4H), 2.89-2.98 (m, 3H), 3.20-3.26 (m, 1H), 3.42-3.50 (m, 1H), 4.26-4.37 (m, 1H), 4.40-4.55 (m, 3H), 5.10-5.16 (m, 1H), 6.36 (s, 1H), 6.86-6.89 (m, 1H), 7.49-7.53 (m, 1H), 7.64 (d, J=7.20 Hz, 1H), 7.71 (d, J=7.20 Hz, 1H), 8.12-8.17 (m, 2H), 8.47 (s, 1H), 8.78-8.79 (m, 1H), 10.22 (s, 1H), 11.00 (br s, 1H), 11.28 (br s, 1H). Mass spec: m/z: 699.0 [M+H]⁺.

### Intermediate 329 was Synthesized Following Scheme 72

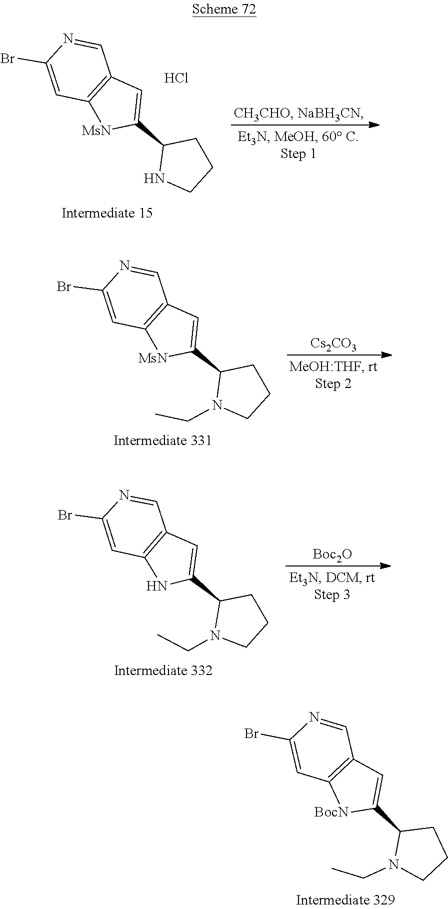

### Step 1

### Intermediate 331: (R)-6-bromo-2-(1-ethylpyrrolidin-2-yl)-1-(methylsulfonyl)-1H-pyrrolo [3,2-c]pyridine

To a cooled (0° C.) solution of (R)-6-bromo-1-(methylsulfonyl)-2-(pyrrolidin-2-yl)-1H-pyrrolo[3,2-c]pyridine hydrochloride (Intermediate 15, 10.0 g, 26.27 mmol) in methanol (50 mL) was added triethylamine (29.4 mL, 210.1 mmol) and acetaldehyde (4.21 mL, 78.80 mmol) followed by sodium cyanoborohydride (3.88 g, 52.53 mmol). The reaction mixture was then heated at 60° C. for 16 h. The reaction mixture was diluted with water (200 mL) and extracted with ethyl acetate (3×200 mL). The combined organic layers were dried over anhydrous Na₂SO₄, filtered, and concentrated in vacuo. The crude material was purified by combi-flash chromatography, by eluting with 60% ethyl acetate in heptane, to afford (R)-6-bromo-2-(1-ethylpyrrolidin-2-yl)-1-(methylsulfonyl)-1H-pyrrolo[3,2-c]pyridine (Intermediate 331, 5.6 g, 57%) as a yellow solid. ¹H NMR (400 MHz, DMSO-d₆) δ ppm 0.97-1.17 (m, 3H), 1.64-1.82 (m, 3H), 2.20-2.38 (m, 3H), 2.72-2.84 (m, 1H), 3.22-3.31 (m, 1H), 3.55 (s, 3H), 3.95-4.04 (m, 1H), 6.92 (s, 1H), 7.96 (s, 1H), 8.67 (s, 1H). Mass spec m/z 373.1 [M+H+2]⁺.

### Step 2

### Intermediate 332: (R)-6-bromo-2-(1-ethylpyrrolidin-2-yl)-1H-pyrrolo[3,2-c]pyridine

To a cooled (0° C.) solution of (R)-6-bromo-2-(1-ethylpyrrolidin-2-yl)-1-(methylsulfonyl)-1H-pyrrolo[3,2-c]pyridine (Intermediate 331, 5.60 g, 15.04 mmol) in tetrahydrofuran (25 mL) and methanol (25 mL) was added cesium carbonate (9.81 g, 30.08 mmol). The reaction mixture was stirred at room temperature for 4 h. The reaction mixture was diluted with water (200 mL) and extracted with ethyl acetate (3×200 mL). The combined organic layers were dried over anhydrous Na₂SO₄, filtered, and concentrated in vacuo to afford crude (R)-6-bromo-2-(1-ethylpyrrolidin-2-yl)-1H-pyrrolo[3,2-c]pyridine (Intermediate 332, 4.1 g, 93%) as an off-white solid which was used for the next step without further purification. ¹H NMR (400 MHz, DMSO-d₆) δ ppm 0.91-0.97 (m, 3H), 1.69-1.88 (m, 3H), 2.06-2.20 (m, 3H), 2.49-2.55 (m, 1H), 3.17-3.21 (m, 1H), 3.44-3.48 (m, 1H), 6.40 (s, 1H), 7.40 (s, 1H), 8.44 (s, 1H), 11.48 (br s, 1H). Mass spec m/z 295.9 [M+H+2]⁺.

### Step 3

### Intermediate 329: tert-butyl-(R)-6-bromo-2-(1-ethylpyrrolidin-2-yl)-1H-pyrrolo[3,2-c]pyridine-1-carboxylate

To a solution of (R)-6-bromo-2-(1-ethylpyrrolidin-2-yl)-1H-pyrrolo[3,2-c]pyridine (Intermediate 332, 4.0 g, 14 mmol) in dichloromethane (40 mL) was added triethylamine (5.7 mL, 41 mmol) followed by di-tert-butyl dicarbonate (6.0 g, 27 mmol) and 4-dimethylaminopyridine (0.34 g, 2.7 mmol) and the reaction mixture was stirred at room temperature for 3 h. The reaction mixture was diluted with water (500 mL) and extracted with DCM (3×500 mL). The combined organic layers were dried over anhydrous Na₂SO₄, filtered, and concentrated in vacuo. The crude material was purified by combi-flash chromatography, by eluting with 45% ethyl acetate in heptane, to afford tert-butyl (R)-6-bromo-2-(1-ethylpyrrolidin-2-yl)-1H-pyrrolo[3,2-c]pyridine-1-carboxylate which was further purified by SFC chiral purification (Column: Chiralpak IK (250*30 mm) 5 um. Isocratic elution with 25% Mobile Phase (A): CO₂ and mobile Phase (B): MeCN+isopropyl alcohol with 0.1% isopropylamine) to afford desired isomer tert-butyl-(R)-6-bromo-2-(1-ethylpyrrolidin-2-yl)-1H-pyrrolo[3,2-c]pyridine-1-carboxylate (Intermediate 329, 2.2 g, 41%) as an off white solid. ¹H NMR (400 MHz, DMSO-d₆) δ ppm 1.06 (t, J=7.13 Hz, 3H), 1.65 (s, 9H), 1.70-1.80 (m, 2H), 2.24-2.38 (m, 4H), 2.72-2.82 (m, 1H), 3.22-3.25 (m, 1H), 4.06-4.10 (m, 1H), 6.83 (s, 1H), 8.05 (s, 1H), 8.61 (s, 1H). Mass spec m/z 395.8 [M+H]⁺.

### Example 74 was Synthesized Following Scheme 73

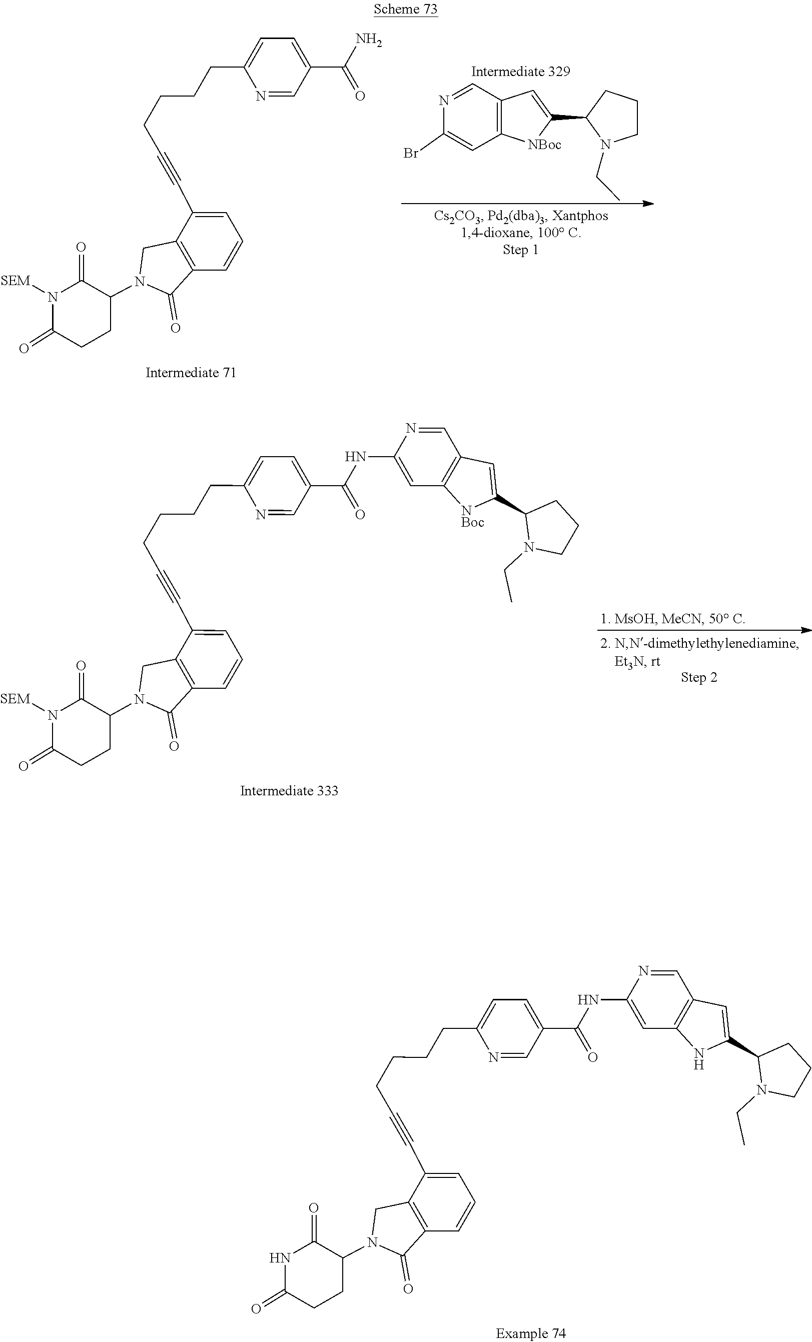 [SMILES: C[Si](C)(C)CCOCN1C(=O)CCC(N2Cc3c(C#CCCCCc4ccc(C(N)=O)cn4)cccc3C2=O)C1=O ; CCN1CCC[C@@H]1c1cc2cnc(Br)cc2n1C(=O)OC(C)(C)C ; CCN1CCC[C@@H]1c1cc2cnc(NC(=O)c3ccc(CCCCC#Cc4cccc5c4CN(C4CCC(=O)N(COCC[Si](C)(C)C)C4=O)C5=O)nc3)cc2n1C(=O)OC(C)(C)C ; CCN1CCC[C@@H]1c1cc2cnc(NC(=O)c3ccc(CCCCC#Cc4cccc5c4CN(C4CCC(=O)NC4=O)C5=O)nc3)cc2[nH]1]

### Step 1

### Intermediate 333: tert-butyl-6-(6-(6-(2-(2,6-dioxo-1-((2-(trimethylsilyl)ethoxy)methyl)piperidin-3-yl)-1-oxoisoindolin-4-yl)hex-5-yn-1-yl)nicotinamido)-2-((R)-1-ethylpyrrolidin-2-yl)-1H-pyrrolo[3,2-c]pyridine-1-carboxylate

To a solution of 6-(6-(2-(2,6-dioxo-1-((2-(trimethylsilyl)ethoxy)methyl)piperidin-3-yl)-1-oxoisoindolin-4-yl)hex-5-yn-1-yl)nicotinamide (Intermediate 71, 0.35 g, 0.61 mmol) in 1,4-dioxane (10 mL) was added tert-butyl-(R)-6-bromo-2-(1-ethylpyrrolidin-2-yl)-1H-pyrrolo[3,2-c]pyridine-1-carboxylate (Intermediate 329, 0.29 g, 0.73 mmol) followed by cesium carbonate (0.59 g, 1.83 mmol). The reaction mixture was purged with argon for 15 min, then Pd₂(dba)₃ (0.086 g, 0.091 mmol) and Xantphos (0.11 g, 0.18 mmol) were added. The reaction mixture was purged with argon for another 10 min and then stirred at 100° C. for 2 h. The reaction mixture was filtered through celite bed, the filter pad was washed with ethyl acetate (100 mL) and the filtrate was concentrated in vacuo. The crude material was purified by combi-flash chromatography, by eluting with 4% MeOH in DCM, to afford tert-butyl-6-(6-(6-(2-(2,6-dioxo-1-((2-(trimethylsilyl)ethoxy)methyl)piperidin-3-yl)-1-oxoisoindolin-4-yl)hex-5-yn-1-yl)nicotinamido)-2-((R)-1-ethylpyrrolidin-2-yl)-1H-pyrrolo[3,2-c]pyridine-1-carboxylate (Intermediate 333, 0.33 g, 61% yield) as a pale yellow solid. ¹H NMR (400 MHz, DMSO-d₆) δ ppm −0.05 (s, 9H), 0.75-0.85 (m, 2H), 1.07 (t, J=6.84 Hz, 3H), 1.50-1.64 (m, 4H), 1.69 (s, 9H), 1.74-1.80 (m, 2H), 1.82-1.95 (m, 2H), 1.99-2.11 (m, 1H), 2.21-2.43 (m, 4H), 2.53-2.58 (m, 1H), 2.72-2.92 (m, 4H), 2.99-3.12 (m, 1H), 3.21-3.26 (m, 1H), 3.44-3.56 (m, 2H), 4.08-4.15 (m, 1H), 4.25-4.30 (m, 1H), 4.45-4.50 (m 1H), 4.96-5.09 (m, 2H), 5.24-5.30 (m, 1H), 6.79 (s, 1H), 7.41 (d, J=7.60 Hz, 1H), 7.51-7.56 (m, 1H), 7.65 (d, J=7.40 Hz, 1H), 7.72 (d, J=7.40 Hz, 1H), 8.27 (d, J=7.60 Hz, 1H), 8.60 (s, 1H), 8.92 (s, 1H), 9.06 (br s, 1H), 10.90 (br s, 1H). Mass spec m/z 888.6 [M+H]⁺.

### Step 2

### Example 74: 6-(6-(2-(2,6-dioxopiperidin-3-yl)-1-oxoisoindolin-4-yl) hex-5-yn-1-yl)-N-(2-((R)-1-ethylpyrrolidin-2-yl)-1H-pyrrolo[3,2-c]pyridin-6-yl) nicotinamide

To a solution of tert-butyl-6-(6-(6-(2-(2,6-dioxo-1-((2-(trimethylsilyl)ethoxy)methyl)piperidin-3-yl)-1-oxoisoindolin-4-yl)hex-5-yn-1-yl)nicotinamido)-2-((R)-1-ethylpyrrolidin-2-yl)-1H-pyrrolo[3,2-c]pyridine-1-carboxylate (Intermediate 333, 0.30 g, 0.34 mmol) in acetonitrile (10 mL) was added methanesulfonic acid (0.22 mL, 3.38 mmol) and the reaction mixture was stirred at 50° C. for 2 h. After cooling to room temperature, N,N′-dimethylethylenediamine (0.40 mL, 3.38 mmol) followed by triethylamine (1.66 mL, 11.82 mmol) were added the reaction mixture was stirred at room temperature for 2 h. The reaction mixture was concentrated in vacuo and the residue was diluted with ice cold water (80 mL). The resultant precipitate was collected by filtration, washed with pentane (2×40 mL) and dried in vacuo. The crude material was purified by preparative HPLC (Method A) to afford 6-(6-(2-(2,6-dioxopiperidin-3-yl)-1-oxoisoindolin-4-yl)hex-5-yn-1-yl)-N-(2-((R)-1-ethylpyrrolidin-2-yl)-1H-pyrrolo[3,2-c]pyridin-6-yl)nicotinamide (Example 74, 0.12 g, 53% yield) as an off white solid. ¹H NMR (400 MHz, DMSO-d₆) δ ppm 0.98 (t, J=7.20 Hz, 3H), 1.58-1.68 (m, 2H), 1.78-1.95 (m, 5H), 1.97-2.06 (m, 1H), 2.10-2.24 (m, 3H), 2.37-2.43 (m, 2H), 2.56-2.62 (m, 3H), 2.85-2.90 (m, 3H), 3.21-3.27 (m, 1H), 3.44-3.50 (m, 1H), 4.28-4.36 (m, 1H), 4.42-4.50 (m, 1H), 5.11-5.16 (m, 1H), 6.38 (s, 1H), 7.40 (d, J=8.00 Hz, 1H), 7.49-7.55 (m, 1H), 7.64 (d, J=7.40 Hz, 1H), 7.71 (d, J=7.40 Hz, 1H), 8.19 (s, 1H), 8.29 (dd, J=8.00, 2.00 Hz, 1H), 8.50 (s, 1H), 9.08 (d, J=2.00 Hz, 1H), 10.66 (s, 1H), 10.99 (br s, 1H), 11.34 (br s, 1H). Mass spec m/z 658.1 [M+H]⁺.

### Example 75 was Synthesized Following Scheme 74

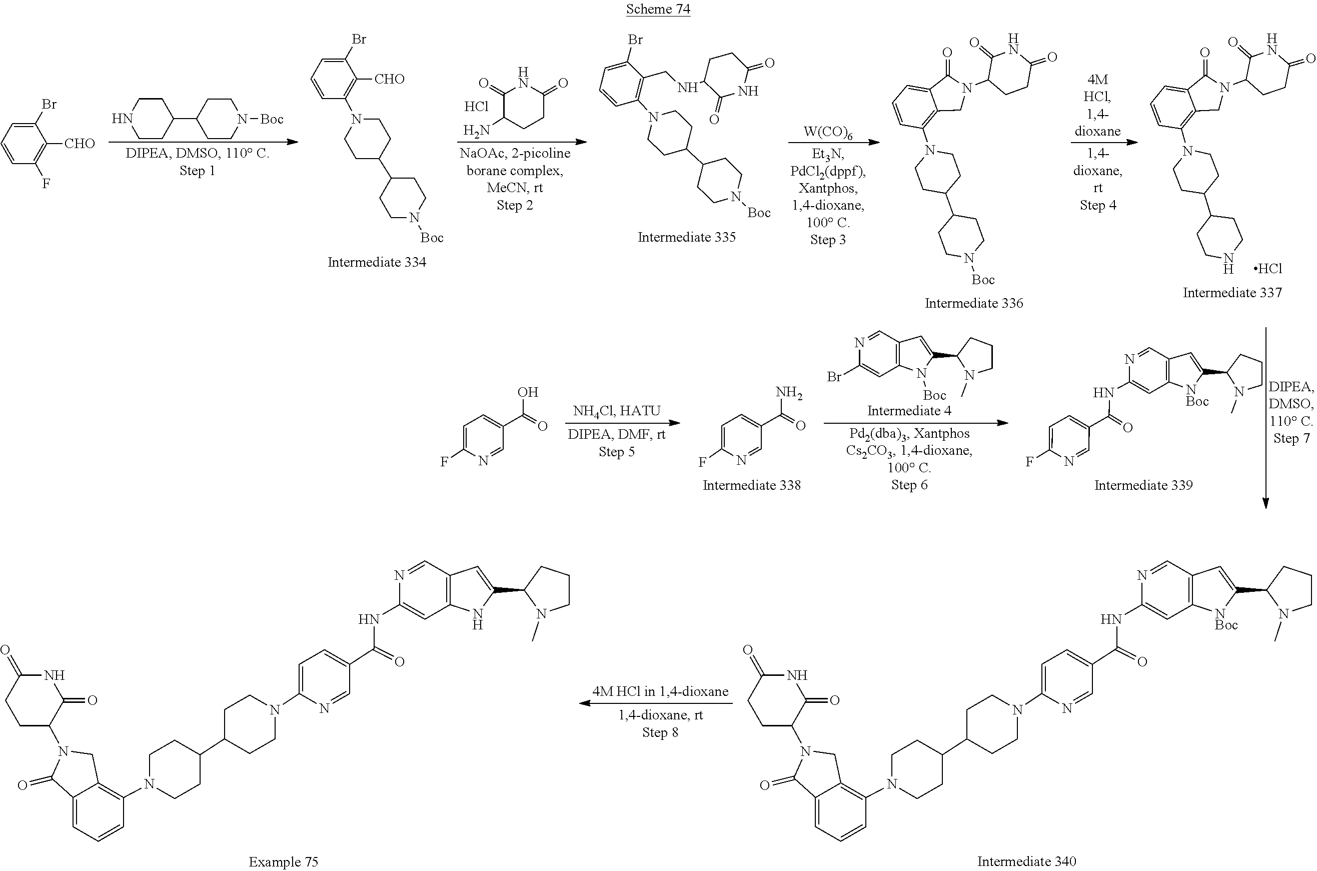

### Step 1

### Intermediate 334: tert-butyl 1′-(3-bromo-2-formylphenyl)-[4,4′-bipiperidine]-1-carboxylate

To a solution of 2-bromo-6-fluorobenzaldehyde (CAS No: 360575-28-6, 4.0 g, 20 mmol) in DMSO (10 mL) was added N,N-diisopropylethylamine (13.0 g, 99 mmol) followed by tert-butyl [4,4′-bipiperidine]-1-carboxylate (CAS No: 171049-35-7, 6.30 g, 24.0 mmol) at room temperature and the reaction mixture was heated to 110° C. for 16 h. The reaction mixture was quenched with water (50 mL), resulting in a precipitate, which was filtered and dried in vacuo to afford tert-butyl 1′-(3-bromo-2-formylphenyl)-[4,4′-bipiperidine]-1-carboxylate (Intermediate 334, 4.0 g, 45%) as a yellow solid which was used for the next step without further purification. ¹H NMR (400 MHz, DMSO-d₆) δ ppm 0.94-1.09 (m, 2H), 1.15-1.24 (m, 2H), 1.38 (s, 9H), 1.60-1.76 (m, 4H), 2.60-2.68 (m, 2H), 2.76-2.84 (m, 2H), 3.12-3.20 (m, 2H), 3.86-4.04 (m, 4H), 7.21 (d, J=8.40 Hz, 1H), 7.31 (d, J=8.40 Hz, 1H), 7.38 (t, J=8.40 Hz, 1H), 10.02 (s, 1H). Mass spec: m/z: 451.3 [M+H]⁺.

### Step 2

### Intermediate 335: tert-butyl 1′-(3-bromo-2-(((2,6-dioxopiperidin-3-yl)amino)methyl)phenyl)-[4,4′-bipiperidine]-1-carboxylate

To a solution of tert-butyl 1′-(3-bromo-2-formylphenyl)-[4,4′-bipiperidine]-1-carboxylate (Intermediate 334, 4.0 g, 8.86 mmol) and 3-aminopiperidine-2,6-dione hydrochloride (CAS No: 24666-56-6, 2.20 g, 13.30 mmol) in acetonitrile (60 mL) was added sodium acetate (1.09 g, 13.30 mmol) followed by 2-picoline borane complex (CAS No: 3999-38-0, 1.90 g, 17.72 mmol) and the reaction mixture was stirred at room temperature for 12 h. The reaction mixture was quenched with water (100 mL) and extracted with ethyl acetate (3×100 mL). The combined organic phases were dried over Na₂SO₄, filtered, and concentrated in vacuo to obtain the crude compound. The crude material was purified by combi-flash chromatography by eluting with EtOAc to afford tert-butyl 1′-(3-bromo-2-(((2,6-dioxopiperidin-3-yl)amino)methyl)phenyl)-[4,4′-bipiperidine]-1-carboxylate (Intermediate 335, 2.0 g, 40%) as a solid. ¹H NMR (400 MHz, DMSO-d₆) δ ppm 0.84-0.88 (m, 1H), 0.97-1.11 (m, 2H), 1.16-1.31 (m, 4H), 1.39 (s, 9H), 1.65-1.81 (m, 4H), 2.24-2.34 (m, 2H), 2.42-2.47 (m, 2H), 2.53-2.58 (m, 1H), 2.64-2.76 (m, 2H), 2.92-2.98 (m, 1H), 3.08-3.16 (m, 1H), 3.20-3.26 (m, 1H), 3.85-4.04 (m, 4H), 7.09-7.20 (m, 2H), 7.32 (d, J=7.60 Hz, 1H), 10.78 (s, 1H). Mass spec: m/z: 563.3 [M+H]⁺.

### Step 3

### Intermediate 336: tert-butyl 1′-(2-(2,6-dioxopiperidin-3-yl)-1-oxoisoindolin-4-yl)-[4,4′-bipiperidine]-1-carboxylate

To a solution of tert-butyl 1′-(3-bromo-2-(((2,6-dioxopiperidin-3-yl)amino)methyl)phenyl)-[4,4′-bipiperidine]-1-carboxylate (Intermediate 335, 2.0 g, 3.60 mmol) in 1,4-dioxane (20 mL) was added triethylamine (1.10 g, 11.0 mmol) and tungsten hexacarbonyl (0.64 g, 1.80 mmol).

The reaction mixture was purged with argon gas for 15 min then Xantphos (0.42 g, 0.71 mmol) followed by PdCl₂(dppf) (0.55 g, 0.71 mmol) were added at room temperature. The reaction mixture was further purged with argon gas for 10 min and then heated at 100° C. for 16 h. The reaction mixture was quenched with water (30 mL) and extracted with ethyl acetate (3×100 mL). The combined organic phases were dried over Na₂SO₄, filtered, and concentrated in vacuo to obtain the crude compound. The crude material was purified by combi-flash chromatography by eluting with 70% EtOAc in heptane to afford tert-butyl 1′-(2-(2,6-dioxopiperidin-3-yl)-1-oxoisoindolin-4-yl)-[4,4′-bipiperidine]-1-carboxylate (Intermediate 336, 1.10 g, 61%) as a brown solid. ¹H NMR (400 MHz, DMSO-d₆) δ ppm 1.00-1.10 (m, 3H), 1.27-1.32 (m, 1H), 1.39 (s, 9H), 1.64-1.79 (m, 4H), 1.95-2.04 (m, 2H), 2.60-2.73 (m, 4H), 2.87-2.98 (m, 2H), 3.11-3.18 (m, 1H), 3.36-3.45 (m, 3H), 3.92-4.04 (m, 2H), 4.24-4.33 (m, 1H), 4.41-4.46 (m, 1H), 5.09-5.14 (m, 1H), 7.15 (d, J=8.0 Hz, 1H), 7.29 (d, J=8.0 Hz, 1H), 7.40 (t, J=8.0 Hz, 1H), 10.98 (s, 1H). Mass spec: m/z: 511.5 [M+H]⁺.

### Step 4

### Intermediate 337: 3-(4-([4,4′-bipiperidin]-1-yl)-1-oxoisoindolin-2-yl)piperidine-2,6-dione hydrochloride

To a solution of tert-butyl 1′-(2-(2,6-dioxopiperidin-3-yl)-1-oxoisoindolin-4-yl)-[4,4′-bipiperidine]-1-carboxylate (Intermediate 336, 1.0 g, 2.0 mmol) in 1,4-dioxane (10 mL) was added 4M HCl in dioxane (20 mL) at 0° C. The reaction mixture was then stirred at room temperature for 16 h under a nitrogen atmosphere. The reaction mixture was concentrated under reduced pressure to afford 3-(4-([4,4′-bipiperidin]-1-yl)-1-oxoisoindolin-2-yl)piperidine-2,6-dione hydrochloride (Intermediate 337, 0.7 g, 80%) as a brown solid, which was used for the next step without further purification. ¹H NMR (400 MHz, DMSO-d₆) δ ppm 1.00-1.10 (m, 3H), 1.27-1.32 (m, 1H), 1.64-1.79 (m, 4H), 1.95-2.04 (m, 2H), 2.60-2.73 (m, 4H), 2.87-2.98 (m, 2H), 3.11-3.18 (m, 1H), 3.36-3.45 (m, 3H), 3.92-4.04 (m, 2H), 4.24-4.33 (m, 1H), 4.41-4.46 (m, 1H), 5.09-5.14 (m, 1H), 7.30 (d, J=8.0 Hz, 1H), 8.71 (t, J=8.0 Hz, 1H), 8.91 (d, J=8.0 Hz, 1H), 10.09 (br s, 2H), 10.99 (s, 1H). Mass spec: m/z: 411.2 [M+H]⁺.

### Step 5

### Intermediate 338: 6-fluoronicotinamide

To a solution of 6-fluoronicotinic acid (CAS No: 403-45-2, 2.0 g, 14.0 mmol) in dimethylformamide (10 mL) was added HATU (8.30 g, 21.0 mmol) and N,N-diisopropylethylamine (5.60 g, 42.0 mmol) at room temperature. After cooling to 0° C., ammonium chloride (2.24 g, 42.0 mmol) was added and the reaction stirred at room temperature for 16 h. The reaction mixture was quenched with water (50 mL), resulting in a precipitated, which was filtered and dried in vacuo to afford 6-fluoronicotinamide (Intermediate 338, 0.85 g, 43%) as a white solid, which was used in the next step without further purification. ¹H NMR (400 MHz, DMSO-d₆) δ 7.27-7.30 (m, 1H), 7.64 (br s, 1H), 8.16 (br s, 1H), 8.36-8.42 (m, 1H), 8.54 (d, J=8.00 Hz, 1H). Mass spec: m/z: 141 [M+H]⁺.

### Step 6

### Intermediate 339: tert-butyl (R)-6-(6-fluoronicotinamido)-2-(1-methylpyrrolidin-2-yl)-1H-pyrrolo[3,2-c]pyridine-1-carboxylate

To a solution of 6-fluoronicotinamide (Intermediate 338, 0.29 g, 2.10 mmol) in 1,4-dioxane (20 mL) was added tert-butyl (R)-6-bromo-2-(1-methylpyrrolidin-2-yl)-1H-pyrrolo[3,2-c]pyridine-1-carboxylate (Intermediate 4, 1.0 g, 2.60 mmol) followed by cesium carbonate (2.60 g, 7.90 mmol) at room temperature. The reaction mixture was purged with argon for 15 min followed by the addition of Pd₂(dba)₃ (0.37 g, 0.39 mmol) and Xantphos (0.47 g, 0.79 mmol). The reaction mixture was further purged with argon for 10 min and then heated at 100° C. for 2 h. The reaction mixture was concentrated in vacuo and the resulting crude material was purified by combi-flash chromatography by eluting with 80% ethyl acetate in heptane to afford tert-butyl (R)-6-(6-fluoronicotinamido)-2-(1-methylpyrrolidin-2-yl)-1H-pyrrolo[3,2-c]pyridine-1-carboxylate (Intermediate 339, 0.8 g, 69%) as a yellow solid. ¹H NMR (400 MHz, DMSO-d₆) δ ppm 1.59-1.65 (m, 2H), 1.69 (s, 9H), 1.72-1.78 (m, 2H), 2.35 (s, 3H), 2.38-2.42 (m, 1H), 3.11-3.18 (m, 1H), 3.88-3.96 (m, 1H), 6.76 (s, 1H), 7.30-7.34 (m, 1H), 8.52-8.58 (m, 1H), 8.61 (s, 1H), 8.86 (s, 1H), 8.92 (s, 1H), 11.07 (s, 1H). Mass spec: m/z: 440.1 [M+H]⁺.

### Step 7

### Intermediate 340: tert-butyl 6-(6-(1′-(2-(2,6-dioxopiperidin-3-yl)-1-oxoisoindolin-4-yl)-[4,4′-bipiperidin]-1-yl)nicotinamido)-2-((R)-1-methylpyrrolidin-2-yl)-1H-pyrrolo[3,2-c]pyridine-1-carboxylate

To a solution of tert-butyl (R)-6-(6-fluoronicotinamido)-2-(1-methylpyrrolidin-2-yl)-1H-pyrrolo[3,2-c]pyridine-1-carboxylate (Intermediate 339, 0.5 g, 1.14 mmol) in DMSO (5.0 mL) was added 3-(4-([4,4′-bipiperidin]-1-yl)-1-oxoisoindolin-2-yl)piperidine-2,6-dione hydrochloride (Intermediate 337, 0.61 g, 1.37 mmol) followed by N,N-diisopropylethylamine (0.75 g, 5.70 mmol) and the reaction mixture was heated at 110° C. for 6 h. The reaction mixture was quenched with water (50 mL), resulting in a precipitate, which was filtered and dried in vacuo to afford tert-butyl 6-(6-(1′-(2-(2,6-dioxopiperidin-3-yl)-1-oxoisoindolin-4-yl)-[4,4′-bipiperidin]-1-yl)nicotinamido)-2-((R)-1-methylpyrrolidin-2-yl)-1H-pyrrolo[3,2-c]pyridine-1-carboxylate (Intermediate 340, 0.53 g, 56% yield, crude) as a yellow solid, which was used in the next step without further purification. ¹H NMR (400 MHz, DMSO-d₆) δ ppm 1.15-1.24 (m, 2H), 1.39 (s, 9H), 1.60-1.70 (m, 2H), 1.72-1.90 (m, 7H), 1.85-1.93 (m, 2H), 1.96-2.03 (m, 1H), 2.09-2.14 (m, 1H), 2.17 (s, 3H), 2.23-2.30 (m, 1H), 2.56-2.63 (m, 1H), 2.65-2.74 (m, 2H), 2.81-2.94 (m, 3H), 3.11-3.18 (m, 1H), 3.26-3.30 (m, 2H), 3.38-3.46 (m, 2H), 4.26-4.34 (m, 1H), 4.40-4.56 (m, 3H), 5.09-5.14 (m, 1H), 6.37 (s, 1H), 6.87 (d, J=8.0 Hz, 1H), 7.15 (d, J=8.0 Hz, 1H), 7.29-7.33 (m, 1H), 7.40-7.45 (m, 1H), 8.13-8.19 (m, 2H), 8.48 (s, 1H), 8.79 (s, 1H), 10.23 (s, 1H), 10.97 (br s, 1H). Mass spec: m/z: 730.44 [M−100+H]⁺.

### Step 8

### Example 75: 6-(1′-(2-(2,6-dioxopiperidin-3-yl)-1-oxoisoindolin-4-yl)-[4,4′-bipiperidin]-1-yl)-N-(2-((R)-1-methylpyrrolidin-2-yl)-1H-pyrrolo[3,2-c]pyridin-6-yl)nicotinamide

To a solution of tert-butyl 6-(6-(1′-(2-(2,6-dioxopiperidin-3-yl)-1-oxoisoindolin-4-yl)-[4,4′-bipiperidin]-1-yl)nicotinamido)-2-((R)-1-methylpyrrolidin-2-yl)-1H-pyrrolo[3,2-c]pyridine-1-carboxylate (Intermediate 340, 0.5 g, 0.60 mmol) in 1,4-dioxane (10 mL) was added 4M HCl in dioxane (15 mL) at 0° C. The reaction mixture was then stirred at room temperature for 16 h under a nitrogen atmosphere. The reaction mixture was concentrated under reduced pressure to obtain the crude residue which was purified by preparative HPLC (Method A) to afford 6-(1′-(2-(2,6-dioxopiperidin-3-yl)-1-oxoisoindolin-4-yl)-[4,4′-bipiperidin]-1-yl)-N-(2-((R)-1-methylpyrrolidin-2-yl)-1H-pyrrolo[3,2-c]pyridin-6-yl)nicotinamide (Example 75, 0.075 g, 17%) as a an off-white solid. ¹H NMR (400 MHz, DMSO-d₆) δ ppm 1.13-1.52 (m, 7H), 1.76-1.84 (m, 4H), 1.85-1.93 (m, 2H), 1.96-2.03 (m, 1H), 2.09-2.14 (m, 1H), 2.16 (s, 3H), 2.23-2.30 (m, 1H), 2.56-2.63 (m, 1H), 2.65-2.74 (m, 2H), 2.81-2.94 (m, 3H), 3.11-3.18 (m, 1H), 3.26-3.30 (m, 2H), 3.38-3.46 (m, 2H), 4.26-4.34 (m, 1H), 4.40-4.56 (m, 3H), 5.09-5.14 (m, 1H), 6.37 (s, 1H), 6.87 (d, J=9.20 Hz, 1H), 7.15 (d, J=8.0 Hz, 1H), 7.29 (d, J=8.0 Hz, 1H), 7.42 (d, J=8.0 Hz, 1H), 8.11 (d, J=9.20 Hz, 1H), 8.16 (s, 1H), 8.48 (s, 1H), 8.79 (d, J=2.25 Hz, 1H), 10.23 (s, 1H), 10.97 (br s, 1H), 11.32 (s, 1H). Mass spec: m/z: 730.1 [M+H]⁺.

### Intermediate 342 was Synthesised Following Scheme 75

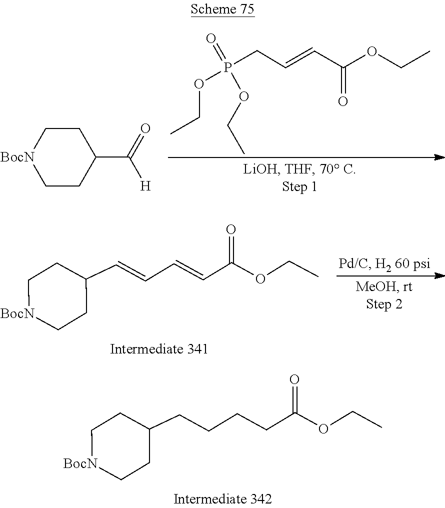

### Step 1

### Intermediate 341: tert-butyl 4-((1E,3E)-5-ethoxy-5-oxopenta-1,3-dien-1-ylpiperidine-1-carboxylate

To a solution of tert-butyl 4-formylpiperidine-1-carboxylate (CAS No: 137076-22-3, 10.0 g, 46.9 mmol) and ethyl (E)-4-(diethoxyphosphoryl)but-2-enoate (CAS No: 42516-28-9, 11.7 g, 46.9 mmol) in tetrahydrofuran (100 mL) was added lithium hydroxide (1.35 g, 56.3 mmol) and the reaction mixture was heated at 80° C. for 2 h. The reaction mixture was diluted with water (50 mL), resulting in a precipitate, which was filtered and dried to obtain crude compound. The crude material was purified by combi-flash column chromatography by eluting with 30% EtOAc in heptane to afford tert-butyl 4-((1E,3E)-5-ethoxy-5-oxopenta-1,3-dien-1-yl)piperidine-1-carboxylate (Intermediate 341, 10.0 g, 70%) as a yellow liquid. Mass spec: m/z: 209.9 [M+H−100]+.

### Step 2

### Intermediate 342: tert-butyl 4-(5-ethoxy-5-oxopentyl)piperidine-1-carboxylate

To a solution of tert-butyl 4-((1E,3E)-5-ethoxy-5-oxopenta-1,3-dien-1-yl)piperidine-1-carboxylate (Intermediate 341, 12.00 g, 40.62 mmol) in methanol (120 mL) was added 10% Pd/C (3.2 g, 30 mmol) at room temperature and the reaction mixture was stirred for 6 h under H₂ atmosphere at 60 psi. The reaction mixture was filtered through celite and concentrated in vacuo. The crude material was purified by combi-flash column chromatography by eluting with 30% EtOAc in heptane to afford tert-butyl 4-(5-ethoxy-5-oxopentyl)piperidine-1-carboxylate (Intermediate 342, 9.6 g, 79%) as a colorless liquid. ¹H NMR (400 MHz, DMSO-d₆) δ ppm 0.87-0.90 (m, 2H), 1.15-1.20 (m, 6H), 1.23-1.31 (m, 1H), 1.38 (s, 9H), 1.46-1.53 (m, 2H), 1.58-1.61 (m, 2H), 2.27 (t, J=7.23 Hz, 2H), 2.62-2.65 (m, 2H), 3.89-3.91 (m, 2H), 4.01-4.06 (m, 2H). Mass spec: m/z: 214.5 [M+H−100]⁺.

### Intermediate 345 was Synthesised Following Scheme 76

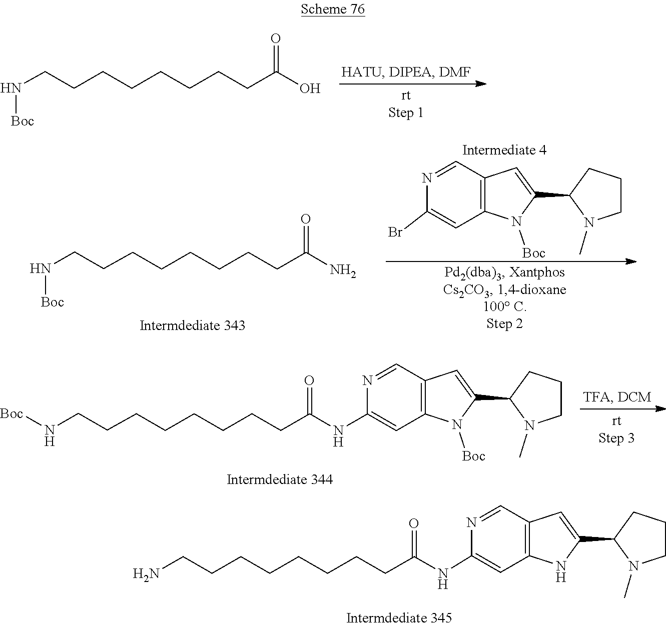 [SMILES: CC(C)(C)OC(=O)NCCCCCCCCC(=O)O ; CC(C)(C)OC(=O)NCCCCCCCCC(N)=O ; CN1CCC[C@@H]1c1cc2cnc(Br)cc2n1C(=O)OC(C)(C)C ; CN1CCC[C@@H]1c1cc2cnc(NC(=O)CCCCCCCCNC(=O)OC(C)(C)C)cc2n1C(=O)OC(C)(C)C ; CN1CCC[C@@H]1c1cc2cnc(NC(=O)CCCCCCCCN)cc2[nH]1]

### Step 1

### Intermediate 343: tert-butyl N-(8-carbamoyloctyl)carbamate

To a stirred solution of 9-[(tert-butoxycarbonyl)amino]nonanoic acid (CAS No: 173435-78-4, 300 mg, 1.09 mmol), HATU (537 mg, 1.41 mmol), DIPEA (702 mg, 5.43 mmol) in anhydrous DMF (6 mL) was added NH₄Cl (174 mg, 3.26 mmo). The reaction was stirred at rt for 2 h and poured into water (50 mL) and filtered. The filter cake was washed with water and dried to get tert-butyl N-(8-carbamoyloctyl)carbamate (Intermediate 343, 240 mg, 73.0%) as a white solid, which was used in the next step without further purification. Mass spec: m/z/: 295.20 [M+Na]+.

### Step 2

### Intermediate 344: N-[8-({2-[(2R)-1-methylpyrrolidin-2-yl]-1H-pyrrolo[3,2-c]pyridin-6-yl}carbamoyl)octyl]carbamate

To a solution of tert-butyl N-(8-carbamoyloctyl)carbamate (Intermediate 343, 100 mg, 0.360 mmol) and tert-butyl 6-bromo-2-[(2R)-1-methylpyrrolidin-2-yl]pyrrolo[3,2-c]pyridine-1-carboxylate (Intermediate 4, 137 mg, 0.327 mmol) in 1,4-dioxane (4 mL) was added Cs₂CO₃ (293 mg, 0.900 mmol), Xantphos (41.6 mg, 0.0720 mmol) and Pd₂(dba)₃ (33.0 mg, 0.0360 mmol) and the reaction was stirred for 3 h at 100° C. under a nitrogen atmosphere. The volatiles were removed under reduced pressure and the crude residue was purified by a C18 silica column, eluting with MeCN/H₂O (37/63) to afford tert-butyl N-[8-({2-[(2R)-1-methylpyrrolidin-2-yl]-1H-pyrrolo[3,2-c]pyridin-6-yl}carbamoyl)octyl]carbamate (Intermediate 344, 120 mg, 70%) as a yellow solid. Mass spec: m/z: 572.25 [M+H]⁺.

### Step 3

### Intermediate 345: 9-amino-N-{2-[(2R)-1-methylpyrrolidin-2-yl]-1H-pyrrolo[3,2-c]pyridin-6-yl}nonanamide ditrifluoroacetate

To a solution of tert-butyl 6-{9-[(tert-butoxycarbonyl)amino]nonanamido}-2-[(2R)-1-methylpyrrolidin-2-yl]pyrrolo[3,2-c]pyridine-1-carboxylate (Intermediate 344, 120 mg, 0.184 mmol) in DCM (2 mL) was added TFA (1 mL) and the reaction was stirred at rt for 2 h. The volatiles were removed under reduced pressure to afford 9-amino-N-{2-[(2R)-1-methylpyrrolidin-2-yl]-1H-pyrrolo[3,2-c]pyridin-6-yl}nonanamide ditrifluoroacetate (Intermediate 345, 80.0 mg, 99.7%) as a brown oil. Mass spec: m/z: 372.25 [M+H]⁺.

### Example 76 was Prepared Following Scheme 77

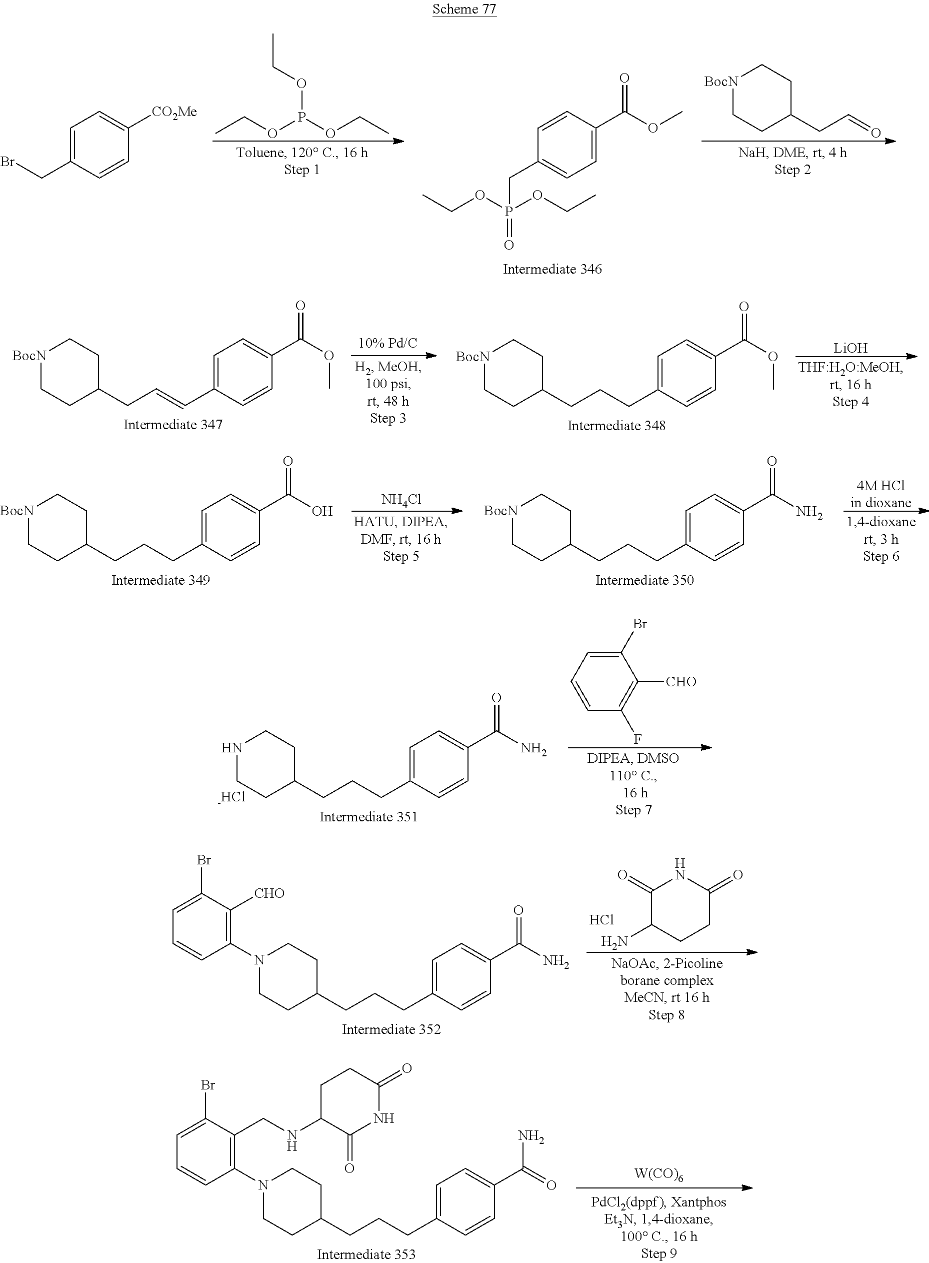

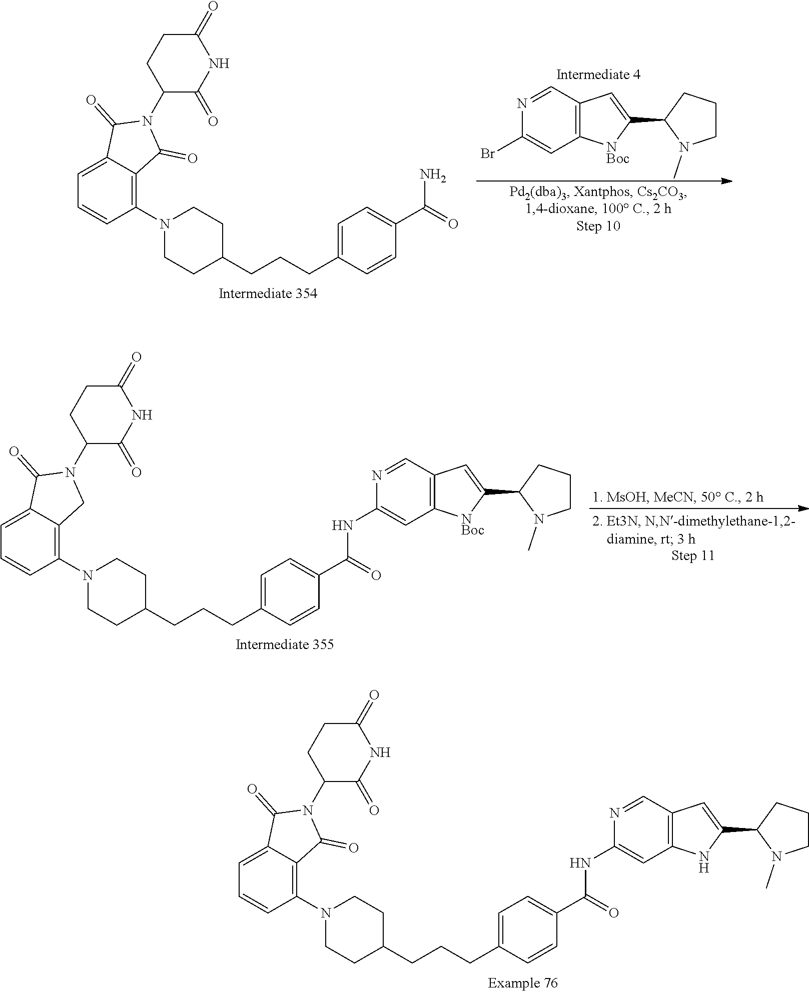 [SMILES: NC(=O)c1ccc(CCCC2CCN(c3cccc4c3C(=O)N(C3CCC(=O)NC3=O)C4=O)CC2)cc1 ; CN1CCC[C@@H]1c1cc2cnc(Br)cc2n1C(=O)OC(C)(C)C ; CN1CCC[C@@H]1c1cc2cnc(NC(=O)c3ccc(CCCC4CCN(c5cccc6c5CN(C5CCC(=O)NC5=O)C6=O)CC4)cc3)cc2n1C(=O)OC(C)(C)C ; CN1CCC[C@@H]1c1cc2cnc(NC(=O)c3ccc(CCCC4CCN(c5cccc6c5C(=O)N(C5CCC(=O)NC5=O)C6=O)CC4)cc3)cc2[nH]1]

### Step 1

### Intermediate 346: methyl 4-((diethoxyphosphoryl)methyl)benzoate

To a solution of methyl 4-(bromomethyl)benzoate (CAS No. 2417-72-3, 5.0 g, 21.8 mmol) in toluene (50 mL) was added triethyl phosphite (CAS No. 122-52-1, 3.62 g, 21.8 mmol) and the reaction mixture was heated at 120° C. for 16 h. After completion, the reaction mixture was concentrated in vacuo to obtain crude compound. The crude material was purified by combi-flash chromatography, by eluting with 20% ethyl acetate in heptane, to afford methyl 4-((diethoxyphosphoryl)methyl)benzoate (Intermediate 346, 4.8 g, 76%) as a light viscous oil. ¹H NMR (400 MHz, DMSO-d₆) δ ppm 1.10-1.15 (m, 6H), 3.32-3.36 (m, 2H), 3.81 (s, 3H), 3.87-3.95 (m, 4H), 7.36-7.40 (m, 2H), 7.87 (d, J=7.89 Hz, 2H). Mass spec m/z 287.2 [M+H]⁺.

### Step 2

### Intermediate 347: tert-butyl-(E)-4-(3-(4-(methoxycarbonyl)phenyl)allyl)piperidine-1-carboxylate

To a solution of methyl 4-((diethoxyphosphoryl)methyl)benzoate (Intermediate 346, 2.0 g, 6.98 mmol) and tert-butyl-4-(2-oxoethyl)piperidine-1-carboxylate (CAS No. 142374-19-4, 1.27 g, 5.58 mmol) in dimethylformamide (40 mL) was added sodium hydride (0.27 g, 6.98 mmol) and the reaction mixture was stirred at room temperature for 4 h. The reaction mixture was quenched with saturated aqueous ammonium chloride solution (100 mL) and extracted with dichloromethane (2×200 mL). The combined organic layers were dried over anhydrous Na₂SO₄ filtered and concentrated in vacuo. The crude material was purified by combi-flash chromatography, by eluting with 30% ethyl acetate in heptane, to afford tert-butyl-(E)-4-(3-(4-(methoxycarbonyl)phenyl)allyl)piperidine-1-carboxylate (Intermediate 347, 1.35 g, 48%) as a yellow liquid. ¹H NMR (400 MHz, DMSO-d₆) δ ppm 0.98-1.02 (m, 2H), 1.34 (s, 9H), 1.51-1.64 (m, 3H), 2.08-2.16 (m, 2H), 2.56-2.72 (m, 2H), 3.80 (s, 3H), 3.87-3,.93 (m, 2H), 6.44 (s, 2H), 7.50 (d, J=8.29 Hz, 2H), 7.85 (d, J=8.29 Hz, 2H). Mass spec m/z 260.1 [M+H-Boc].

### Step 3

### Intermediate 348: tert-butyl-4-(3-(4-(methoxycarbonyl)phenyl)propyl)piperidine-1-carboxylate

To a solution of tert-butyl-(E)-4-(3-(4-(methoxycarbonyl)phenyl)allyl)piperidine-1-carboxylate (Intermediate 347, 2.7 g, 7.5 mmol) in methanol (50 mL) was added 10% Pd/C (1.0 g, 4.69 mmol). The reaction mixture was stirred at room temperature under an atmosphere of hydrogen at 100 psi for 48 h. After completion, the reaction mixture was filtered through a celite bed, the filter pad was washed with DCM (100 mL) and MeOH (100 mL). The filtrate was concentrated in vacuo to afford tert-butyl-4-(3-(4-(methoxycarbonyl)phenyl)propyl)piperidine-1-carboxylate (Intermediate 348, 2.5 g, 66%) as an off-white solid, which was used for the next step without further purification. ¹H NMR (400 MHz, DMSO-d₆) δ ppm 0.86-0.90 (m, 2H), 1.16-1.23 (m, 2H), 1.34 (s, 9H), 1.35-1.38 (m, 1H), 1.55-1.58 (m, 4H), 2.58-2.67 (m, 4H), 3.79 (s, 3H), 3.84-3.87 (m, 2H), 7.31 (d, J=7.83 Hz, 2H), 7.84 (d, J=8.22 Hz, 2H). Mass spec m/z 362.2 [M+H]⁺.

### Step 4

### Intermediate 349: 4-(3-(1-(tert-butoxy carbonyl)piperidin-4-yl)propyl)benzoic acid

To a solution of tert-butyl-4-(3-(4-(methoxycarbonyl)phenyl)propyl)piperidine-1-carboxylate (Intermediate 348, 2.4 g, 6.6 mmol) in tetrahydrofuran: methanol: water (2:2:1, 25 mL) was added lithium hydroxide (0.65 g, 27.0 mmol). The reaction mixture was stirred at room temperature for 16 h then concentrated in vacuo to one-third volume. The pH was adjusted the ~5 by the addition of 1N aqueous HCl solution. The resultant precipitate was collected by filtration, washed with heptane (200 mL), and dried in vacuo to afford 4-(3-(1-(tert-butoxycarbonyl)piperidin-4-yl)propyl)benzoic acid (Intermediate 349, 2.3 g) as an off-white solid, which was used for the next step without further purification. ¹H NMR (400 MHz, DMSO-d₆) δ ppm 0.87-0.97 (m, 2H), 1.16-1.24 (m, 2H), 1.37 (s, 9H), 1.53-1.66 (m, 4H), 2.58-2.69 (m, 3H), 3.82-3.96 (m, 2H), 7.31 (d, J=7.88 Hz, 2H), 7.85 (d, J=8.29 Hz, 2H), 12.73 (br s, 1H). Mass spec m/z 346.2 [M−H]⁻.

### Step 5

### Intermediate 350: tert-butyl-4-(3-(4-carbamoylphenyl)propyl)piperidine-1-carboxylate

To a solution of 4-(3-(1-(tert-butoxy carbonyl) piperidin-4-yl)propyl)benzoic acid (Intermediate 349, 2.3 g, 6.6 mmol) in dimethylformamide (15 mL) was added HATU (4.0 g, 9.9 mmol) and the reaction mixture was stirred at room temperature for 15 min. Ammonium chloride (1.8 g, 33.0 mmol), followed by N,N-diisopropylethylamine (3.5 mL, 20.0 mmol) were added and the reaction mixture was stirred at room temperature for 16 h. The reaction mixture was poured into ice cold water (150 mL), the resultant precipitate was collected by filtration and dried in vacuo to afford tert-butyl-4-(3-(4-carbamoylphenyl)propyl)piperidine-1-carboxylate (Intermediate 350, 1.6 g, 70%) as an off-white solid, which was used for next step without further purification. ¹H NMR (400 MHz, DMSO-d₆) δ ppm 0.85-0.98 (m, 2H), 1.14-1.25 (m, 2H), 1.37 (s, 9H), 1.52-1.66 (m, 4H), 2.58-2.69 (m, 4H), 3.85-3.95 (m, 2H), 7.25 (d, J=7.43 Hz, 4H), 7.78 (d, J=7.83 Hz, 2H), 7.87 (br s, 1H). Mass spec m/z 345.2 [M−H]⁻.

### Step 6

### Intermediate 351: 4-(3-(piperidin-4-yl)propyl)benzamide hydrochloride

To a solution of tert-butyl-4-(3-(4-carbamoylphenyl)propyl)piperidine-1-carboxylate (Intermediate 350, 1.6 g, 4.6 mmol) in 1,4-dioxane (15 mL) was added 4M hydrochloric acid in 1,4-dioxane (15 mL). The reaction mixture was stirred at room temperature for 3 h. The reaction mixture was concentrated in vacuo to afford 4-(3-(piperidin-4-yl)propyl)benzamide hydrochloride (Intermediate 351, 1.30 g) as an off-white solid, which was used for the next step without further purification. ¹H NMR (400 MHz, DMSO-d₆) δ ppm 1.16-1.35 (m, 4H), 1.49-1.63 (m, 3H), 1.75-1.80 (m, 2H), 2.57-2.65 (m, 2H), 2.73-2.86 (m, 2H), 3.18-3.24 (m, 2H), 7.25-7.32 (m, 3H), 7.79 (d, J=8.19 Hz, 2H), 7.91 (br s, 1H), 8.51 (br s, 1H), 8.72 (br s, 1H). Mass spec m/z 245.1 [M−H]⁻.

### Step 7

### Intermediate 352: 4-(3-(1-(3-bromo-2-formylphenyl)piperidin-4-yl)propyl)benzamide

To a solution of 4-(3-(piperidin-4-yl)propyl)benzamide hydrochloride (Intermediate 351, 1.20 g, 4.24 mmol) and 2-bromo-6-fluorobenzaldehyde (CAS No. 360575-28-6, 0.86 g, 4.24 mmol) in dimethyl sulfoxide (10 mL) was added N,N-diisopropylethylamine (3.69 mL, 21.2 mmol).

The reaction mixture was heated at 110° C. for 16 h then diluted with water (300 mL) and extracted with ethyl acetate (3×100 mL). The combined organic layers were dried over anhydrous Na₂SO₄, filtered and concentrated in vacuo. The crude material was purified by combi-flash chromatography, by eluting with 60% ethyl acetate in heptane, to afford 4-(3-(1-(3-bromo-2-formylphenyl)piperidin-4-yl)propyl)benzamide (Intermediate 352, 0.66 g, 36%) as a yellow solid. ¹H NMR (400 MHz, DMSO-d₆) δ ppm 1.27-1.39 (m, 6H), 1.57-1.68 (m, 3H), 1.70-1.78 (m, 2H), 2.61-2.65 (m, 2H), 3.13-3.16 (m, 2H), 7.20 (d, J=8.22 Hz, 1H), 7.23-7.33 (m, 4H), 7.38-7.42 (m, 1H), 7.79 (d, J=7.83 Hz, 2H), 7.88 (br s, 1H), 10.01 (br s, 1H). Mass spec m/z 431.0 [M+H+2]f.

### Step 8

### Intermediate 353: 4-(3-(1-(3-bromo-2-(((2,6-dioxopiperidin-3-yl)amino)methyl)phenyl)-piperidin-4-yl)-propyl)benzamide

To a cooled (0° C.) solution of 4-(3-(1-(3-bromo-2-formylphenyl)-piperidin-4-yl)-propyl)benzamide (Intermediate 352, 0.65 g, 1.51 mmol) and 3-aminopiperidine-2,6-dione hydrochloride (CAS No. 24666-56-6, 0.27, 1.66 mmol) in acetonitrile (12 mL) was added sodium acetate (0.18 g, 2.27 mmol) followed by 2-picoline borane complex (CAS No. 999-38-0, 0.42 g, 3.78 mmol). The reaction mixture was stirred at room temperature for 16 h then quenched with water (100 mL) and extracted with ethyl acetate (2×100 mL). The combined organic layers were dried over Na₂SO₄, filtered, and concentrated in vacuo to obtain the crude compound. The crude material was purified by combi-flash chromatography, by eluting with 50% ethyl acetate in heptane, to afford 4-(3-(1-(3-bromo-2-(((2,6-dioxopiperidin-3-yl)amino)methyl)phenyl)-piperidin-4-yl)-propyl)benzamide (Intermediate 353, 0.70 g) as an off-white solid, which was used for the next step without further purification. Mass spec m/z 543.1 [M+H]⁺.

### Step 9

### Intermediate 354: 4-(3-(1-(2-(2,6-dioxopiperidin-3-yl)-1-oxoisoindolin-4-yl)piperidin-4-yl)-propyl)benzamide

To a solution of 4-(3-(1-(3-bromo-2-(((2,6-dioxopiperidin-3-yl)amino)methyl)phenyl)piperidin-4-yl)-propyl)benzamide (Intermediate 353, 0.60 g, 1.10 mmol) in 1,4-dioxane (10 mL) was added triethylamine (0.46 mL, 3.32 mmol) and tungsten hexacarbonyl (0.20 g, 0.55 mmol). The reaction mixture was purged with argon gas for 15 min then Xantphos (0.13 g, 0.22 mmol) and Pd(dppf)Cl₂ (0.17 g, 0.22 mmol) were added at room temperature. The reaction mixture was further purged with argon gas for 10 min and heated at 100° C. for 16 h, then concentrated in vacuo. The crude material was purified by combi-flash chromatography, by eluting with 50% ethyl acetate in heptane, to afford 4-(3-(1-(2-(2,6-dioxopiperidin-3-yl)-1-oxoisoindolin-4-yl)-piperidin-4-yl)-propyl)benzamide (Intermediate 354, 0.50 g, 92%) as a white solid. Mass spec m/z 489.5 [M+H]⁺.

### Step 10

### Intermediate 355: tert-butyl-6-(4-(3-(1-(2-(2,6-dioxopiperidin-3-yl)-1-oxoisoindolin-4-yl)piperidin-4-yl) propyl)benzamido)-2-((R)-1-methylpyrrolidin-2-yl)-1H-pyrrolo[3,2-c]pyridine-1-carboxylate

To a solution of 4-(3-(1-(2-(2,6-dioxopiperidin-3-yl)-1-oxoisoindolin-4-yl)piperidin-4-yl)propyl)benzamide (Intermediate 354, 0.30 g, 0.61 mmol) in 1,4-dioxane (10 mL) was added tert-butyl-(R)-6-bromo-2-(1-methylpyrrolidin-2-yl)-1H-pyrrolo[3,2-c]pyridine-1-carboxylate (Intermediate 4, 0.2 g, 0.73 mmol) followed by cesium carbonate (0.50 g, 1.53 mmol) at room temperature. The reaction mixture was purged with argon for 15 min and then Pd₂(dba)₃ (0.08 g, 0.09 mmol) and Xantphos (0.10 g, 0.18 mmol) were added. The reaction mixture was purged with argon for another 10 min and stirred at 100° C. for 2 h. The reaction mixture was then filtered through a celite bed and the filter pad was washed with ethyl acetate (500 mL). The filtrate was concentrated in vacuo and the crude material purified by combi-flash chromatography, by eluting with 90% ethyl acetate in heptane, to afford tert-butyl-6-(4-(3-(1-(2-(2,6-dioxopiperidin-3-yl)-1-oxoisoindolin-4-yl)piperidin-4-yl)propyl)benzamido)-2-((R)-1-methylpyrrolidin-2-yl)-1H-pyrrolo[3,2-c]pyridine-1-carboxylate (Intermediate 355, 0.37 g, 76%) as a yellow solid. ¹H NMR (400 MHz, DMSO-d₆) δ ppm 1.07-1.10 (m, 2H), 1.23-1.40 (m, 7H), 1.58-1.66 (m, 2H), 1.69 (s, 9H), 1.72-1.81 (m, 4H), 1.95-2.02 (m, 1H), 2.36 (s, 3H), 2.63-2.75 (m, 4H), 2.87-2.94 (m, 1H), 3.10-3.17 (m, 1H), 3.34-3.42 (m, 3H), 3.87-3.96 (m, 1H), 4.23-4.29 (m, 1H), 4.39-4.43 (m, 1H), 5.08-5.12 (m, 1H), 6.75 (s, 1H), 7.15 (d, J=7.43 Hz, 1H), 7.27-7.34 (m, 3H), 7.38-7.45 (m, 1H), 7.98 (d, J=7.43 Hz, 2H), 8.58 (s, 1H), 8.92 (s, 1H), 10.58 (br s, 1H), 10.96 (br s, 1H). Mass spec m/z 786.4 [M−H]⁻.

### Step 11

### Example 76: 4-(3-(1-(2-(2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide

To a solution of tert-butyl 6-(4-(3-(1-(2-(2,6-dioxopiperidin-3-yl)-1-oxoisoindolin-4-yl)piperidin-4-yl)propyl)benzamido)-2-((R)-1-methylpyrrolidin-2-yl)-1H-pyrrolo[3,2-c]pyridine-1-carboxylate (Intermediate 355, 0.33 g, 0.41 mmol) in acetonitrile (6 mL) was added methanesulfonic acid (0.27 mL, 4.18 mmol) and the reaction mixture was heated at 50° C. for 2 h. The reaction mixture was cooled to room temperature, then N,N′-dimethylethylenediamine (0.25 mL, 2.09 mmol) followed by triethylamine (1.17 mL, 8.37 mmol) were added and the reaction mixture was stirred at room temperature for 3 h. The reaction mixture was concentrated in vacuo and the resultant residue was diluted with water (50 mL).

The resultant precipitate was collected by filtration and dried in vacuo. The crude solid was purified by preparative HPLC (Method A) to afford 4-(3-(1-(2-(2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide (Example 76, 0.08 g, 27%) as an off-white solid. ¹H NMR (400 MHz, DMSO-d₆) δ ppm 1.25-1.37 (m, 4H), 1.40-1.51 (m, 1H), 1.64-1.78 (m, 2H), 1.80-1.86 (m, 3H), 1.87-2.01 (m, 3H), 2.13-2.17 (m, 1H), 2.18 (s, 3H), 2.23-2.32 (m, 1H), 2.46-2.50 (m, 2H), 2.55-2.63 (m, 2H), 2.68-2.72 (m, 2H), 2.73-2.78 (m, 1H), 2.87-2.99 (m, 1H), 3.12-3.20 (m, 1H), 3.36-3.43 (m, 2H), 4.27-4.31 (m, 1H), 4.39-4.47 (m, 1H), 5.10-5.14 (m, 1H), 6.40 (s, 1H), 7.17 (d, J=7.88 Hz, 1H), 7.31 (d, J=7.25 Hz, 1H), 7.35 (d, J=8.25 Hz, 2H), 7.40-7.47 (m, 1H), 8.00 (d, J=8.25 Hz, 2H), 8.20 (s, 1H), 8.51 (s, 1H), 10.37 (br s, 1H), 10.98 (br s, 1H), 11.37 (br s, 1H). Mass spec m/z 688.1 [M+H]⁺.

### Examples 77 and 78 was Synthesised Following Scheme 78

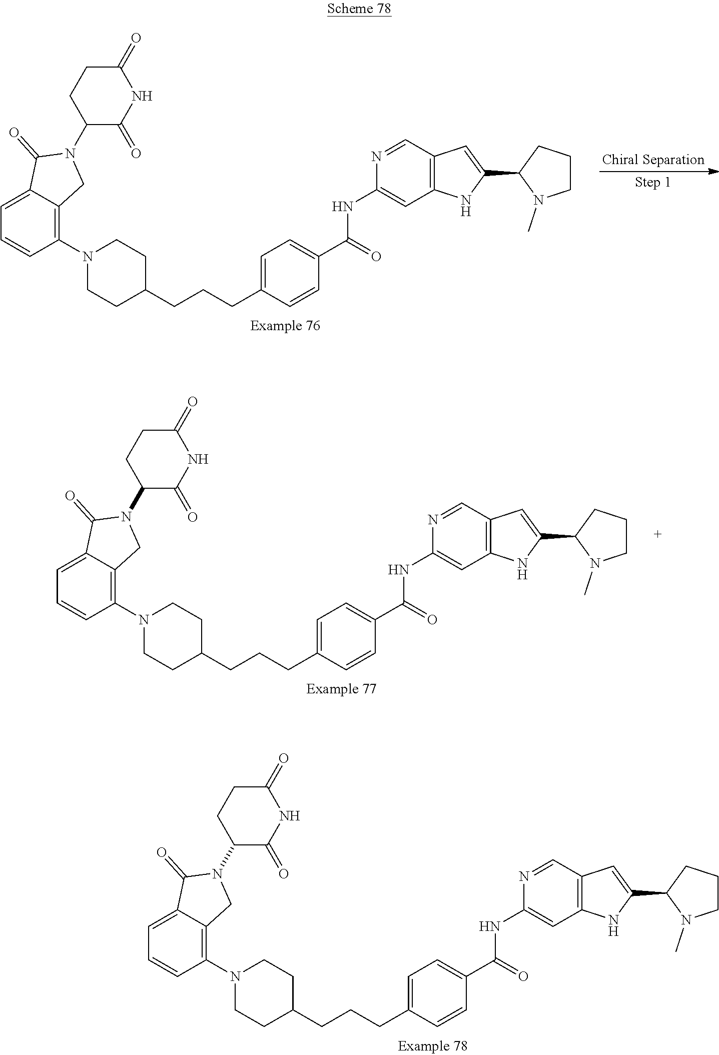

### Step 1

### Example 77: 4-(3-(1-(2-((rel-S)-2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide and Example 78: 4-(3-(1-(2-((rel-R)-2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide

4-(3-(1-(2-(2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide (Example 76, 0.3 g, 0.38 mmol) was separated by chiral preparative HPLC (Column: RR WHELK (30×250*mm), 5 m; Mobile Phase A: DCM, Mobile Phase B: iPrOH; Flow rate: 42 ml/min; isocratic 60% B) to afford 4-(3-(1-(2-((rel-S)-2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide (Example 77, 1st eluting peak, 0.02 g, 8%) as an off-white solid and 4-(3-(1-(2-((rel-R)-2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide (Example 78, 2nd eluting peak, 0.09 g, 34%) as an off-white solid.

### Example 77: 4-(3-(1-(2-((rel-S)-2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide

1^{st} eluting peak from chiral preparative HPLC. Retention time: 5.84 mins.

¹H NMR (400 MHz, DMSO-d₆) δ ppm 1.23-1.33 (m, 4H), 1.37-1.48 (m, 1H), 1.59-1.73 (m, 2H), 1.72-1.85 (m, 3H), 1.85-1.94 (m, 1H), 1.94-2.02 (m, 1H), 2.08-2.14 (m, 1H), 2.17 (s, 3H), 2.21-2.31 (m, 1H), 2.55-2.63 (m, 2H), 2.63-2.77 (m, 4H), 2.84-2.97 (m, 1H), 3.09-3.19 (m, 1H), 3.25-3.30 (m, 2H), 3.34-3.39 (m, 2H), 4.23-4.33 (m, 1H), 4.37-4.47 (m, 1H), 5.08-5.12 (m, 1H), 6.39 (s, 1H), 7.15 (d, J=8.00 Hz, 1H), 7.29 (d, J=7.13 Hz, 1H), 7.33 (d, J=8.25 Hz, 2H), 7.38-7.43 (m, 1H), 7.98 (d, J=8.25 Hz, 2H), 8.19 (s, 1H), 8.50 (s, 1H), 10.34 (br s, 1H), 10.95 (s, 1H), 11.35 (br s, 1H). Mass spec m/z 688.1 [M+H]⁺.

### Example 78: 4-(3-(1-(2-((rel-R)-2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide

2nd eluting peak from chiral preparative HPLC. Retention time: 7.29 mins.

¹H NMR (400 MHz, DMSO-d₆) δ ppm 1.22-1.33 (m, 5H), 1.38-1.49 (m, 1H), 1.61-1.73 (m, 2H), 1.74-1.82 (m, 3H), 1.84-1.92 (m, 2H), 1.94-2.01 (m, 1H), 2.11-2.15 (m, 1H), 2.16 (s, 3H), 2.20-2.31 (m, 1H), 2.55-2.63 (m, 1H), 2.64-2.74 (m, 4H), 2.85-2.96 (m, 1H), 3.10-3.18 (m, 1H), 3.27-3.30 (m, 1H), 3.34-3.42 (m, 2H), 4.23-4.32 (m, 1H), 4.36-4.46 (m, 1H), 5.08-5.12 (m, 1H), 6.38 (s, 1H), 7.15 (d, J=7.75 Hz, 1H), 7.29 (d, J=7.13 Hz, 1H), 7.33 (d, J=8.38 Hz, 2H), 7.38-7.46 (m, 1H), 7.98 (d, J=8.25 Hz, 2H), 8.18 (s, 1H), 8.49 (s, 1H), 10.35 (br s, 1H), 10.96 (br s, 1H), 11.35 (br s, 1H). Mass spec m/z 688.3 [M+H]⁺.

### Example 79 was Prepared Following Scheme 79

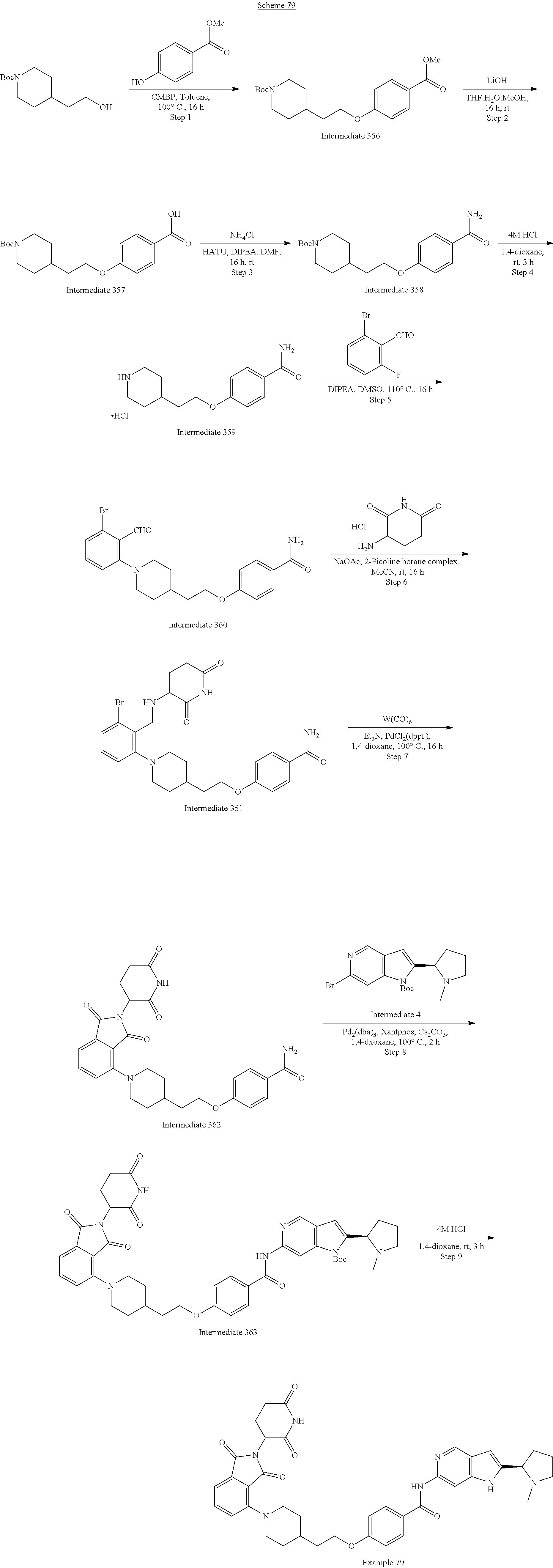

Step 1

### Intermediate 356: tert-butyl 4-(2-(4-(methoxycarbonyl)phenoxy)ethyl)piperidine-1-carboxylate

To a solution of methyl 4-hydroxybenzoate (CAS No. 99-76-3, 4.97 g, 32.7 mmol) and tert-butyl 4-(2-hydroxyethyl)piperidine-1-carboxylate (CAS No. 89151-44-0, 5.0 g, 21.8 mmol) in toluene (50 mL) was added (cyanomethylene)tributylphosphorane (CMBP, CAS No. 157141-27-0, 16.3 g, 65.4 mmol) and the reaction mixture was heated at 100C for 16 h. The reaction mixture was diluted with water (500 mL) and extracted with ethyl acetate (6×100 mL). The combined organic layers were dried over anhydrous Na₂SO₄, filtered, and concentrated in vacuo. The crude material was purified by combi-flash chromatography, by eluting with 40% ethyl acetate in heptane, to afford tert-butyl 4-(2-(4-(methoxycarbonyl)phenoxy)ethyl)piperidine-1-carboxylate (Intermediate 356, 6.0 g, 76%) as a brown solid. ¹H NMR (400 MHz, DMSO-d₆) δ ppm 0.85-0.90 (m, 3H), 1.01-1.07 (m, 1H), 1.39 (s, 9H), 1.53-1.69 (m, 5H), 2.65-2.69 (m, 2H), 3.80 (s, 3H), 3.87-3.94 (m, 1H), 4.05-4.12 (m, 1H), 7.03 (d, J=8.8 Hz, 2H), 7.89 (d, J=8.4 Hz, 2H). Mass spec m/z 264.1 [M−100+H]⁺.

### Step 2

### Intermediate 357: 4-(2-(1-(tert-butoxycarbonyl)piperidin-4-yl)ethoxy)benzoic acid

To a solution of tert-butyl 4-(2-(4-(methoxycarbonyl)phenoxy)ethyl)piperidine-1-carboxylate (Intermediate 356, 7.0 g, 19.3 mmol) in methanol (30 mL), tetrahydrofuran (30 mL) and water (15 mL) was added lithium hydroxide (1.88 g, 77.1 mmol) and the reaction mixture was stirred at room temperature for 16 h. The reaction mixture was then concentrated in vacuo, diluted with water (500 mL) and the pH was adjusted to 3-4 with 0.5N aqueous HCl solution. The resultant precipitate was collected by filtration and dried in vacuo to afford 4-(2-(1-(tert-butoxycarbonyl)piperidin-4-yl)ethoxy)benzoic acid (Intermediate 357, 4.5 g, 67%) as a white solid, which was used for the next step without further purification. ¹H NMR (400 MHz, DMSO-d₆) δ ppm 1.01-1.07 (m, 2H), 1.38 (s, 9H), 1.63-1.67 (m, 5H), 2.65-2.71 (m, 2H), 3.85-3.93 (m, 2H), 4.05-4.08 (m, 2H), 6.99 (d, J=8.8 Hz, 2H), 7.85 (d, J=8.4 Hz, 2H), 12.28 (br s, 1H). Mass spec m/z 250.3 [M−100+H]⁺.

### Step 3

### Intermediate 358: tert-butyl 4-(2-(4-carbamoylphenoxy)ethyl)piperidine-1-carboxylate

To a solution of 4-(2-(1-(tert-butoxycarbonyl)piperidin-4-yl)ethoxy)benzoic acid (Intermediate 357, 4.40 g, 13.0 mmol) in dimethylformamide (50 mL) was added HATU (7.40 g, 19.0 mmol) and N,N-diisopropylethylamine (6.60 mL, 38.0 mmol) followed by ammonium chloride (3.40 g, 63.0 mmol). The reaction mixture was stirred at room temperature for 16 h. The reaction mixture was diluted with ice cold water (250 mL), the resultant precipitate was collected by filtration and dried in vacuo to afford tert-butyl 4-(2-(4-carbamoylphenoxy)ethyl)piperidine-1-carboxylate (Intermediate 358, 4.0 g, 91%) as an off-white solid, which was used for the next step without further purification. ¹H NMR (400 MHz, DMSO-d₆) δ ppm 1.02-1.07 (m, 2H), 1.39 (s, 9H), 1.65-1.70 (m, 5H), 2.60-2.74 (m, 2H), 3.89-3.94 (m, 2H), 4.05-4.08 (m, 2H), 6.96 (d, J=8.8 Hz, 2H), 7.15 (br s, 1H), 7.75-7.80 (m, 3H). Mass spec m/z 349.1 [M+H]⁺.

### Step 4

### Intermediate 359: 4-(2-(piperidin-4-yl)ethoxy)benzamide hydrochloride

To a solution of tert-butyl 4-(2-(4-carbamoylphenoxy)ethyl)piperidine-1-carboxylate (Intermediate 358, 4.0 g, 11.5 mmol) in 1,4-dioxane (40 mL) was added 4M hydrochloric acid in 1,4-dioxane (40 mL). The reaction mixture was stirred at room temperature for 3 h. The reaction mixture was concentrated in vacuo to afford 4-(2-(piperidin-4-yl)ethoxy)benzamide hydrochloride (Intermediate 359, 3.70 g) as an off-white solid, which was used for the next step without further purification. ¹H NMR (400 MHz, DMSO-d₆) δ ppm 1.26-1.45 (m, 3H), 1.67-1.87 (m, 5H), 2.75-2.87 (m, 2H), 2.98-3.23 (m, 2H), 4.05-4.08 (m, 2H), 6.96 (d, J=8.40 Hz, 2H), 7.15 (br s, 1H), 7.83 (d, J=8.80 Hz, 2H), 8.79 (br s, 1H), 9.01 (br s, 1H). Mass spec m/z 249.2 [M+H]⁺.

### Step 5

### Intermediate 360: 4-(2-(1-(3-bromo-2-formylphenyl)piperidin-4-yl)ethoxy)benzamide

To a solution of 4-(2-(piperidin-4-yl)ethoxy)benzamide hydrochloride (Intermediate 359, 3.60 g, 12.6 mmol) in dimethyl sulfoxide (40 mL) was added N,N-diisopropylethylamine (11.0 mL, 63.2 mmol) followed by 2-bromo-6-fluoro-benzaldehyde (CAS No. 360575-28-6, 3.85 g, 19.0 mmol) and the reaction mixture was heated at 110° C. for 16 h. The reaction mixture was diluted with water (350 mL) and extracted with ethyl acetate (3×100 mL). The combined organic layers were dried over anhydrous Na₂SO₄, filtered and concentrated in vacuo. The crude material was purified by combi-flash chromatography, by eluting with 60% ethyl acetate in heptane, to afford 4-(2-(1-(3-bromo-2-formyl-phenyl)-4-piperidyl)ethoxy)benzamide (Intermediate 360, 2.80 g, 51%) as a yellow solid. ¹H NMR (400 MHz, DMSO-d₆) δ ppm 1.35-1.46 (m, 2H), 1.60-1.87 (m, 5H), 2.80-2.89 (m, 2H), 3.13-3.18 (m, 2H), 4.05-4.12 (m, 2H), 6.98 (d, J=8.80 Hz, 2H), 7.15 (br s, 1H), 7.21 (d, J=8.0 Hz, 1H), 7.30 (d, J=7.60 Hz, 1H), 7.41 (t, J=8.40 Hz, 1H), 7.80-7.85 (m, 3H), 10.04 (s, 1H). Mass spec m/z 431.0 [M+H]⁺.

### Step 6

### Intermediate 361: 4-(2-(1-(3-bromo-2-(((2,6-dioxopiperidin-3-yl)amino)methyl)phenyl)piperidin-4-yl)ethoxy)benzamide

To a solution of 4-(2-(1-(3-bromo-2-formylphenyl)piperidin-4-yl)ethoxy)benzamide (Intermediate 360, 3.0 g, 6.95 mmol) and 3-aminopiperidine-2,6-dione hydrochloride (CAS No. 24666-56-6, 1.14 g, 6.95 mmol) in acetonitrile (30 mL) were added sodium acetate (1.15 g, 13.9 mmol) and 2-picoline borane complex (2.34 g, 20.9 mmol). The reaction mixture was stirred at room temperature for 16 h then filtered through a celite bed. The filter pad was washed with ethyl acetate (300 mL) and the filtrate concentrated in vacuo. The crude material was purified by combi-flash chromatography, by eluting with 8% MeOH in DCM, to afford 4-(2-(1-(3-bromo-2-(((2,6-dioxopiperidin-3-yl)amino)methyl)phenyl)piperidin-4-yl)ethoxy)benzamide (Intermediate 361, 0.45 g, 11%) as a yellow solid. Mass spec m/z 545.0 [M+H+2]⁺.

### Step 7

### Intermediate 362: 4-(2-(1-(2-(2,6-dioxopiperidin-3-yl)-1-oxoisoindolin-4-yl)piperidin-4-yl)ethoxy)benzamide

To a solution of 4-(2-(1-(3-bromo-2-(((2,6-dioxopiperidin-3-yl)amino)methyl)phenyl)piperidin-4-yl)ethoxy)benzamide (Intermediate 361, 0.45 g, 0.82 mmol) in 1,4-dioxane (10 mL) was added triethylamine (0.34 mL, 2.48 mmol). The reaction mixture was purged with argon for 15 min then Pd(dppf)Cl₂ (0.12 g, 0.16 mmol) followed by Xantphos (0.09 g, 0.16 mmol) and W(CO)₆ (0.15 g, 0.41 mmol) were added. The reaction mixture was further purged with argon for 10 min and heated at 100° C. for 16 h. The reaction mixture was filtered through a celite bed, the filter pad was washed with ethyl acetate (100 mL) and the filtrate concentrated in vacuo. The crude material was purified by combi-flash chromatography, by eluting with 9% MeOH in DCM, to afford 4-(2-(1-(2-(2,6-dioxopiperidin-3-yl)-1-oxoisoindolin-4-yl)piperidin-4-yl)ethoxy)benzamide (Intermediate 362, 0.33 g, 81%) as yellow solid. Mass spec m/z 491.0 [M+H]⁺.

### Step 8

### Intermediate 363: tert-butyl 6-(4-(2-(1-(2-(2,6-dioxopiperidin-3-yl)-1-oxoisoindolin-4-yl)piperidin-4-yl)ethoxy)benzamido)-2-((R)-1-methylpyrrolidin-2-yl)-1H-pyrrolo[3,2-c]pyridine-1-carboxylate

To a solution of tert-butyl (R)-6-bromo-2-(1-methylpyrrolidin-2-yl)-1H-pyrrolo[3,2-c]pyridine-1-carboxylate (Intermediate 4, 0.30 g, 0.81 mmol) and 4-(2-(1-(2-(2,6-dioxopiperidin-3-yl)-1-oxoisoindolin-4-yl)piperidin-4-yl)ethoxy)benzamide (Intermediate 362, 0.33 g, 0.67 mmol) in 1,4-dioxane (30 mL) was added cesium carbonate (0.65 g, 2.01 mmol). The reaction mixture was purged with argon for 15 min then Xantphos (0.12 g, 0.20 mmol) and Pd₂(dba)₃ (0.09 g, 0.10 mmol) were added. The reaction mixture was further purged with argon for 10 min and heated at 100° C. for 2 h. The reaction mixture was filtered through a celite bed, and the filter pad was washed with ethyl acetate (200 mL). The filtrate was concentrated in vacuo and the crude material was purified by combi-flash chromatography, by eluting with 8% MeOH in DCM, to afford tert-butyl 6-(4-(2-(1-(2-(2,6-dioxopiperidin-3-yl)-1-oxoisoindolin-4-yl)piperidin-4-yl)ethoxy)benzamido)-2-((R)-1-methylpyrrolidin-2-yl)-1H-pyrrolo[3,2-c]pyridine-1-carboxylate (Intermediate 363, 0.18 g, 34%) as a yellow solid. Mass spec m/z 790.3 [M+H]⁺.

### Step 9

### Example 79: 4-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide

To a cooled (0° C.) solution of tert-butyl 6-(4-(2-(1-(2-(2,6-dioxopiperidin-3-yl)-1-oxoisoindolin-4-yl)piperidin-4-yl)ethoxy)benzamido)-2-((R)-1-methylpyrrolidin-2-yl)-1H-pyrrolo[3,2-c]pyridine-1-carboxylate (Intermediate 363, 0.18 g, 0.22 mmol) in 1,4-dioxane (3 mL) was added 4M hydrochloric acid in 1,4-dioxane (3 mL) and the reaction mixture was stirred at room temperature for 3 h. The reaction mixture was then concentrated in vacuo and the crude material was purified by preparative HPLC (Method A) to afford 4-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide (Example 79, 0.03 g, 19%) as an off-white solid. ¹H NMR (400 MHz, DMSO-d₆) δ ppm 1.35-1.47 (m, 2H), 1.62-1.71 (m, 1H), 1.72-2.03 (m, 8H), 2.12-2.19 (m, 4H), 2.22-2.29 (m, 1H), 2.43-2.48 (m, 2H), 2.53-2.60 (m, 1H), 2.62-2.69 (m, 2H), 2.87-2.94 (m, 1H), 3.14-3.18 (m, 1H), 3.33-3.42 (m, 2H), 4.13-4.18 (m, 2H), 4.27-4.32 (m, 1H), 4.41-4.47 (m, 1H), 5.09-5.14 (m, 1H), 6.39 (s, 1H), 7.04 (d, J=8.80 Hz, 2H), 7.17 (d, J=8.80 Hz, 1H), 7.30 (d, J=7.20 Hz, 1H), 7.43 (t, J=7.6 Hz, 1H), 8.04 (d, J=8.80 Hz, 2H), 8.17 (s, 1H), 8.49 (s, 1H), 10.25 (s, 1H) 10.98 (s, 1H), 11.32 (s, 1H). Mass spec m/z 690.5 [M+H]⁺.

### Examples 80 and 81 were prepared following Scheme 80

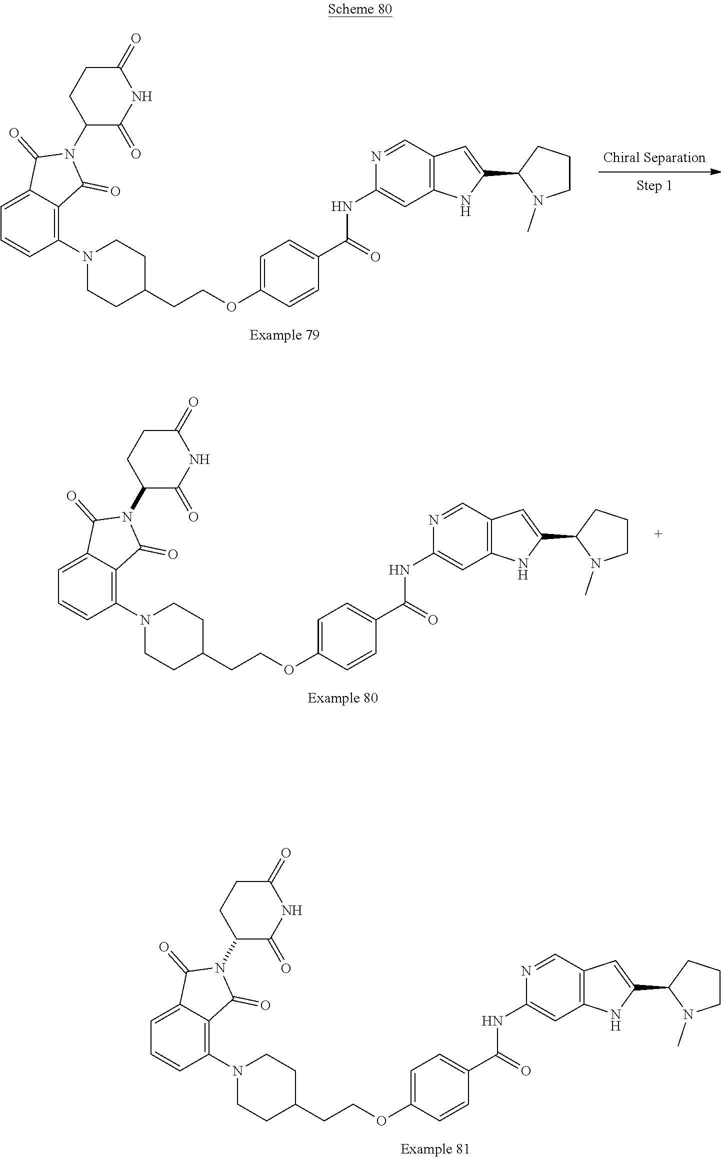

### Step 1

### Example 80: 4-(2-(1-(2-((rel-S)-2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide and Example 81: 4-(2-(1-(2-((rel-R)-2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide

4-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide (Example 79, 0.40 g, 0.57 mmol) was separated by chiral preparative HPLC (CHIRALPAK IK (50×250*mm), 5 m; Mobile Phase A: DCM, Mobile Phase B: DCM: iProH (1:1); Flow rate: 90 ml/min; isocratic 50% B) to afford 4-(2-(1-(2-((rel-S)-2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide (Example 80, 1st eluting peak, 0.12 g, 30%) as an off-white solid and 4-(2-(1-(2-((rel-R)-2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide (Example 81, 2nd eluting peak, 0.08 g, 21%) as an off-white solid.

### Example 80: 4-(2-(1-(2-((rel-S)-2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide

1^{st} eluting peak from chiral preparative HPLC. Retention time: 5.98 mins.

¹H NMR (400 MHz, DMSO-d₆) δ ppm 1.37-1.40 (m, 2H), 1.65-1.71 (m, 1H), 1.72-1.81 (m, 3H), 1.81-1.94 (m, 5H), 1.95-2.01 (m, 1H), 2.12-2.16 (m, 1H), 2.17 (s, 3H), 2.24-2.31 (m, 1H), 2.53-2.63 (m, 2H), 2.67-2.79 (m, 2H), 2.87-2.96 (m, 1H), 3.13-3.16 (m, 1H), 3.34-3.43 (m, 2H), 4.13-4.16 (m, 2H), 4.28-4.31 (m, 1H), 4.42-4.46 (m, 1H), 5.09-5.13 (m, 1H), 6.39 (s, 1H), 7.04 (d, J=8.88 Hz, 2H), 7.17 (d, J=7.88 Hz, 1H), 7.30 (d, J=7.25 Hz, 1H), 7.43 (t, J=7.69 Hz, 1H), 8.05 (d, J=8.88 Hz, 2H), 8.17 (s, 1H), 8.49 (s, 1H), 10.28 (br s, 1H), 10.98 (br s, 1H), 11.35 (br s, 1H). Mass spec m/z 690.4 [M+H]⁺.

### Example 81: 4-(2-(1-(2-((rel-R)-2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide

2nd eluting peak from chiral preparative HPLC. Retention time: 7.50 mins.

¹H NMR (400 MHz, DMSO-d₆) δ ppm 1.32-1.44 (m, 2H), 1.62-1.72 (m, 1H), 1.71-1.81 (m, 3H), 1.80-1.92 (m, 5H), 1.96-2.03 (m, 1H), 2.11-2.15 (m, 1H), 2.17 (s, 3H), 2.57-2.61 (m, 2H), 2.71-2.79 (m, 3H), 2.86-2.95 (m, 1H), 3.13-3.18 (m, 1H), 3.34-3.44 (m, 2H), 4.13-4.16 (m, 2H), 4.28-4.35 (m, 1H), 4.42-4.48 (m, 1H), 5.09-5.13 (m, 1H), 6.40 (s, 1H), 7.04 (d, J=8.75 Hz, 2H), 7.18 (d, J=7.88 Hz, 1H), 7.30 (d, J=7.38 Hz, 1H), 7.41-7.45 (m, 1H), 8.05 (d, J=8.76 Hz, 2H), 8.17 (s, 1H), 8.50 (s, 1H), 10.29 (br s, 1H), 10.98 (br s, 1H), 11.36 (br s, 1H). Mass spec m/z 690.4 [M+H]⁺.

### Example 82 was Prepared Following Scheme 81

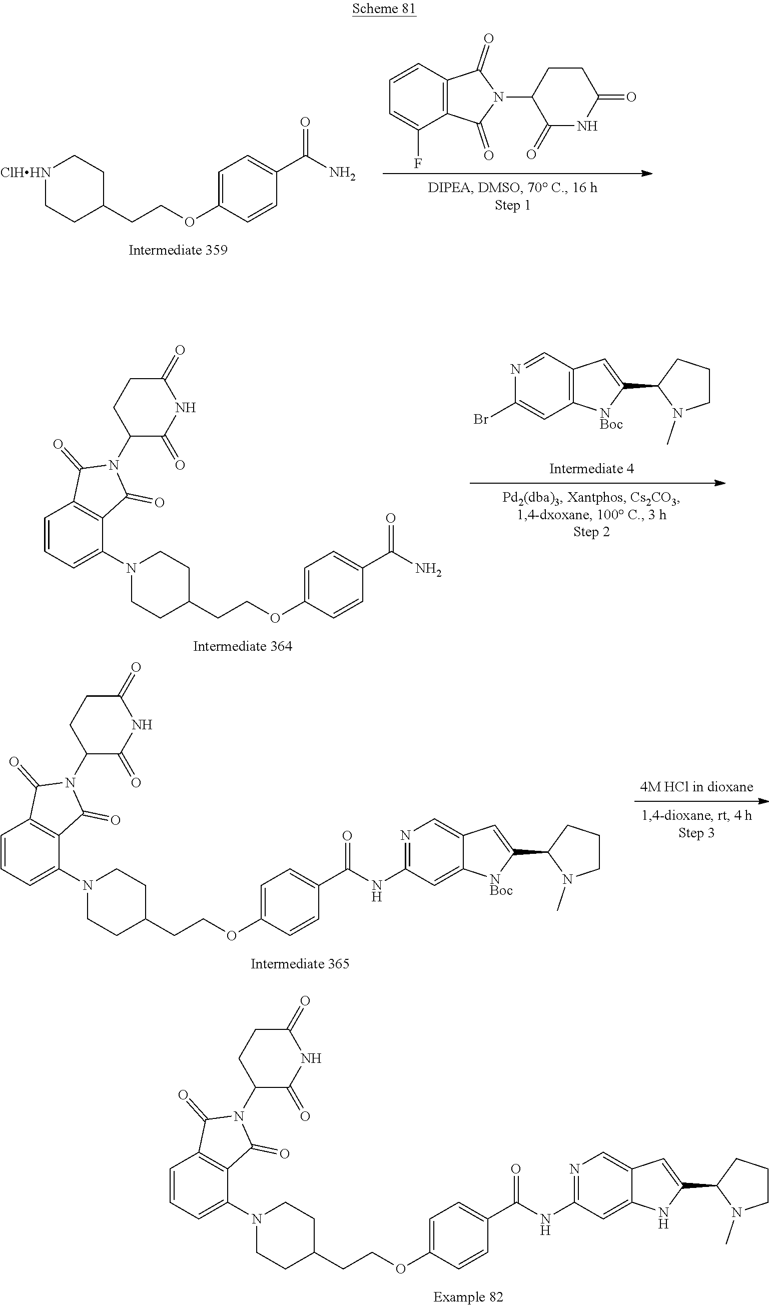

### Step 1

### Intermediate 364: 4-(2-(1-(2-(2,6-dioxopiperidin-3-yl)-1,3-dioxoisoindolin-4-yl)piperidin-4-yl)ethoxy)benzamide

To a solution of 4-(2-(piperidin-4-yl)ethoxy)benzamide hydrochloride (Intermediate 359, 2.0 g, 7.02 mmol) and 2-(2,6-dioxopiperidin-3-yl)-4-fluoroisoindoline-1,3-dione (CAS No: 835616-60-9, 1.94 g, 7.02 mmol) in dimethyl sulfoxide (20 mL) was added N,N-diisopropylethylamine (4.91 mL, 28.1 mmol) and the reaction mixture was heated at 70° C. for 16 h. After completion, the reaction mixture was poured into ice cold water (100 mL), and the resultant precipitate collected by filtration and dried in vacuo to afford 4-(2-(1-(2-(2,6-dioxopiperidin-3-yl)-1,3-dioxoisoindolin-4-yl)piperidin-4-yl)ethoxy)benzamide (Intermediate 364, 2.2 g) as a brown solid, which was used for the next step without further purification. ¹H NMR (400 MHz, DMSO-d₆) δ ppm 1.39-1.45 (m, 2H), 1.70-1.78 (m, 3H), 1.83-1.86 (m, 2H), 1.98-2.07 (m, 2H), 2.84-2.90 (m, 4H), 3.66-3.74 (m, 2H), 4.09-4.15 (m, 2H), 5.06-5.11 (m, 1H), 6.99 (d, J=8.77 Hz, 2H), 7.17 (br s, 1H), 7.33 (t, J=7.45 Hz, 2H), 7.64-7.71 (m, 1H), 7.79-7.84 (m, 2H), 7.85 (br s, 1H), 11.09 (br s, 1H). Mass spec m/z 505.1 [M+H]⁺.

### Step 2

### Intermediate 365: tert-butyl 6-(4-(2-(1-(2-(2,6-dioxopiperidin-3-yl)-1,3-dioxoisoindolin-4-yl) piperidin-4-yl) ethoxy)benzamido)-2-((R)-1-methylpyrrolidin-2-yl)-1H-pyrrolo[3,2-c]pyridine-1-carboxylate

To a solution of 4-(2-(1-(2-(2,6-dioxopiperidin-3-yl)-1,3-dioxoisoindolin-4-yl) piperidin-4-yl) ethoxy)benzamide (Intermediate 364, 1.0 g, 2.0 mmol) in 1,4-dioxane (12 mL) was added tert-butyl-(R)-6-bromo-2-(1-methylpyrrolidin-2-yl)-1H-pyrrolo[3,2-c]pyridine-1-carboxylate (Intermediate 4, 0.83 g, 2.2 mmol) followed by cesium carbonate (1.1 g, 5.9 mmol). The reaction mixture was purged with argon for 15 min and then Pd₂(dba)₃ (0.25 g, 0.30 mmol) and Xantphos (0.35 g, 0.59 mmol) were added. The reaction mixture was further purged with argon for another 10 min and then heated at 100° C. for 3 h. The reaction mixture was concentrated in vacuo. The crude material was purified by combi-flash chromatography, by eluting with 10% MeOH in DCM, to afford tert-butyl 6-(4-(2-(1-(2-(2,6-dioxopiperidin-3-yl)-1,3-dioxoisoindolin-4-yl)piperidin-4-yl)ethoxy)benzamido)-2-((R)-1-methylpyrrolidin-2-yl)-1H-pyrrolo[3,2-c]pyridine-1-carboxylate (Intermediate 365, 1.1 g, 33%) as a yellow solid. ¹H NMR (400 MHz, DMSO-d₆) δ ppm 1.34-1.52 (m, 2H), 1.56-1.64 (m, 2H), 1.69 (s, 9H), 1.71-1.80 (m, 5H), 1.85-1.88 (m, 2H), 2.35 (s, 3H), 2.53-2.56 (m, 4H), 2.85-2.91 (m, 3H), 3.09-3.17 (m, 1H), 3.66-3.72 (m, 2H), 3.88-3.96 (m, 1H), 4.11-4.19 (m, 2H), 5.04-5.13 (m, 1H), 6.75 (s, 1H), 7.05 (d, J=8.61 Hz, 2H), 7.33 (t, J=7.04 Hz, 3H), 8.05 (d, J=8.61 Hz, 2H), 8.58 (s, 1H), 8.92 (s, 1H), 10.53 (br s, 1H), 11.09 (br s, 1H). Mass spec m/z 804.4 [M+H]⁺.

### Step 3

### Example 82: 4-(2-(1-(2-(2,6-dioxopiperidin-3-yl)-1,3-dioxoisoindolin-4-yl)piperidin-4-yl)ethoxy)-N-(2-((R)-1-methylpyrrolidin-2-yl)-1H-pyrrolo[3,2-c]pyridin-6-yl)benzamide

To a cooled (0° C.) solution of tert-butyl-6-(4-(2-(1-(2-(2,6-dioxopiperidin-3-yl)-1,3-dioxoisoindolin-4-yl)piperidin-4-yl) ethoxy)benzamido)-2-((R)-1-methylpyrrolidin-2-yl)-1H-pyrrolo[3,2-c]pyridine-1-carboxylate (Intermediate 365, 0.50 g, 0.62 mmol) in 1,4-dioxane (5 mL) was added 4M hydrochloric acid in 1,4-dioxane (5 mL) and reaction mixture was stirred at room temperature for 4 h. The reaction was concentrated in vacuo. The obtained residue was triturated with diethyl ether (20 mL) and dried in vacuo. The crude material was purified by preparative HPLC (Method A) to afford 4-(2-(1-(2-(2,6-dioxopiperidin-3-yl)-1,3-dioxoisoindolin-4-yl)piperidin-4-yl)ethoxy)-N-(2-((R)-1-methylpyrrolidin-2-yl)-1H-pyrrolo[3,2-c]pyridin-6-yl)benzamide (Example 82, 0.09 g, 22%) as an off-yellow solid. ¹H NMR (400 MHz, DMSO-d₆) δ ppm 1.40-1.49 (m, 2H), 1.72-1.88 (m, 8H), 2.01-2.07 (m, 1H), 2.11-2.15 (m, 1H), 2.16 (s, 3H), 2.22-2.29 (m, 1H), 2.40-2.47 (m, 2H), 2.53-2.58 (m, 1H), 2.82-2.93 (m, 3H), 3.11-3.17 (m, 1H), 3.68-3.73 (m, 2H), 4.14-4.17 (m, 2H), 5.07-5.11 (m, 1H), 6.38 (s, 1H), 7.04 (d, J=8.88 Hz, 2H), 7.29-7.38 (m, 2H), 7.68 (dd, J=8.38, 7.13 Hz, 1H), 8.05 (d, J=8.88 Hz, 2H), 8.17 (s, 1H), 8.49 (s, 1H), 10.27 (br s, 1H), 11.08 (m, 1H), 11.34 (br s, 1H). Mass spec m/z 704.2 [M+H]⁺.

### Example 83 was Prepared Following Scheme 82

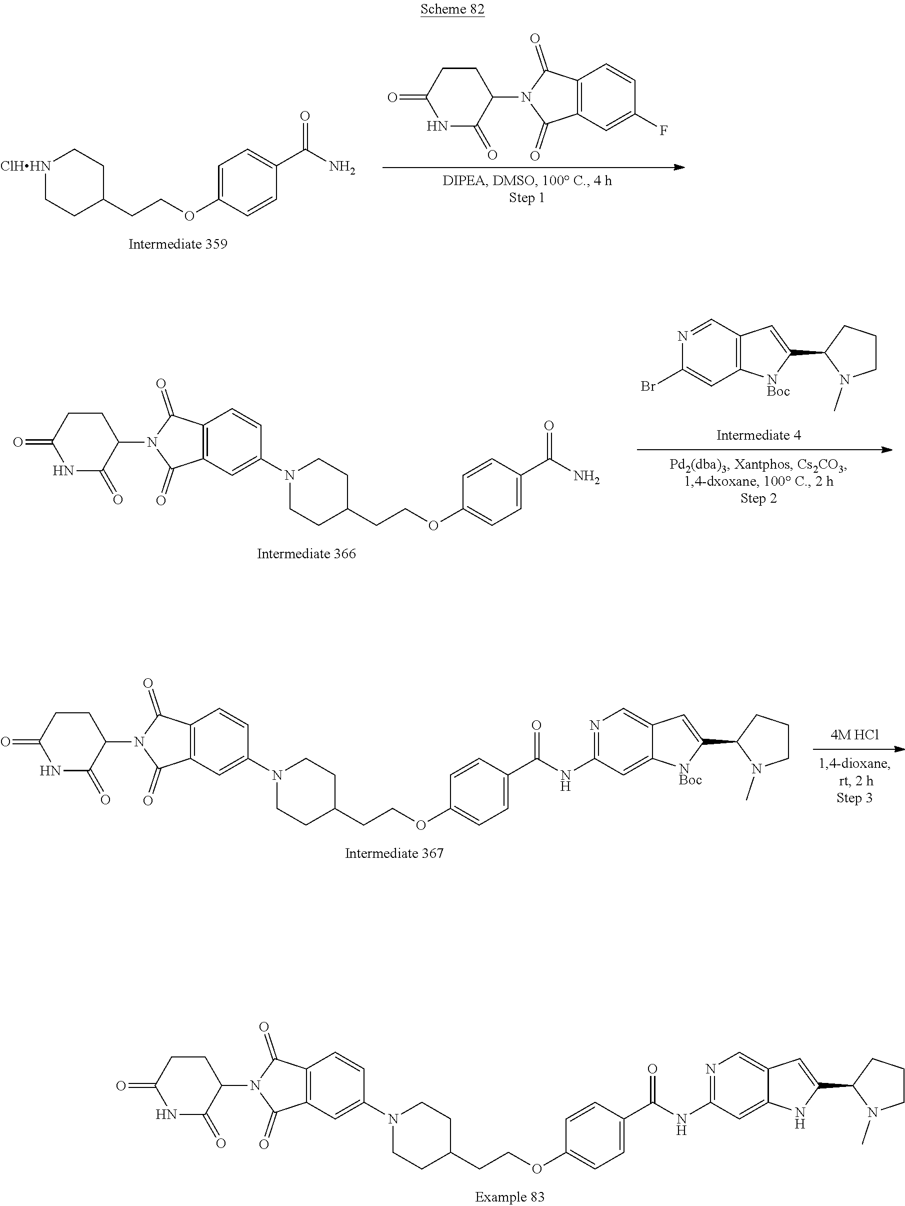

### Step 1

### Intermediate 366: 4-(2-(1-(2-(2,6-dioxopiperidin-3-yl)-1,3-dioxoisoindolin-5-yl)piperidin-4-yl)ethoxy)benzamide

To a solution of 4-(2-(piperidin-4-yl)ethoxy)benzamide hydrochloride (Intermediate 359, 2.0 g, 7.022 mmol) in dimethyl sulfoxide (20 mL) was added N,N-diisopropylethylamine (4.91 mL, 28.09 mmol) followed by 2-(2,6-dioxopiperidin-3-yl)-5-fluoroisoindoline-1,3-dione (CAS No: 835616-61-0, 1.94 g, 7.022 mmol) and the reaction mixture was heated to 100° C. for 4 h. The reaction mixture was quenched with water (200 mL) and extracted with ethyl acetate (2×100 mL). The combined organic layers were dried over Na₂SO₄, filtered and concentrated in vacuo. The crude material was purified by combi-flash chromatography, by eluting with 80% ethyl acetate in heptane, to afford 4-(2-(1-(2-(2,6-dioxopiperidin-3-yl)-1,3-dioxoisoindolin-5-yl)piperidin-4-yl)ethoxy)benzamide (Intermediate 366, 1.50 g, 42%) as an off-white solid. ¹H NMR (400 MHz, DMSO-d₆) δ ppm 1.15-1.23 (m, 1H), 1.42-1.47 (m, 2H), 1.63-1.78 (m, 3H), 1.82-1.85 (m, 2H), 1.99-2.06 (m, 1H), 2.52-2.61 (m, 1H), 2.71-2.82 (m, 3H), 3.67-3.72 (m, 2H), 4.10-4.14 (m, 2H), 5.07-5.11 (m, 1H), 6.98 (d, J=8.80 Hz, 2H), 7.17 (s, 1H), 7.33 (t, J=6.80 Hz, 2H), 7.68 (t, J=7.2 Hz, 1H), 7.76-7.84 (m, 3H), 11.54 (s, 1H). Mass spec m/z 505.3 [M+H]⁺.

### Step 2

### Intermediate 367: tert-butyl 6-(4-(2-(1-(2-(2,6-dioxopiperidin-3-yl)-1,3-dioxoisoindolin-5-yl)piperidin-4-yl)ethoxy)benzamido)-2-((R)-1-methylpyrrolidin-2-yl)-1H-pyrrolo[3,2-c]pyridine-1-carboxylate

To a solution of 4-(2-(1-(2-(2,6-dioxopiperidin-3-yl)-1,3-dioxoisoindolin-5-yl)piperidin-4-yl)ethoxy)benzamide (Intermediate 366, 0.500 g, 0.991 mmol) and tert-butyl (R)-6-bromo-2-(1-methylpyrrolidin-2-yl)-1H-pyrrolo[3,2-c]pyridine-1-carboxylate (Intermediate 4, 0.452 g, 1.189 mmol) in 1,4-dioxane (6 mL) was added cesium carbonate (0.970 g, 2.973 mmol). The reaction mixture was purged with argon gas for 15 min, then Xantphos (0.177 g, 0.297 mmol) and Pd₂(dba)₃ (0.140 g, 0.148 mmol) were added. The reaction mixture was further purged with argon gas for 10 min and heated at 100° C. for 2 h. The reaction mixture was then filtered through a celite bed, and the filter pad was washed with ethyl acetate (200 mL). The filtrate was concentrated in vacuo and the crude material purified by combi-flash chromatography, by eluting with ethyl acetate, to afford 6-(4-(2-(1-(2-(2,6-dioxopiperidin-3-yl)-1,3-dioxoisoindolin-5-yl)piperidin-4-yl)ethoxy)benzamido)-2-((R)-1-methylpyrrolidin-2-yl)-1H-pyrrolo[3,2-c]pyridine-1-carboxylate (Intermediate 367, 0.400 g, 50%) as an off-white solid. ¹H NMR (400 MHz, DMSO-d₆) δ ppm 1.30-1.41 (m, 2H), 1.38-1.49 (m, 2H), 1.61-1.78 (m, 10H), 1.81-1.89 (m, 3H), 1.98-2.05 (m, 3H), 2.31-2.38 (m, 6H), 2.54-2.58 (m, 2H), 2.82-2.92 (m, 3H), 3.10-3.15 (m, 2H), 3.67-3.75 (m, 2H), 3.88-3.92 (m, 1H), 5.08-5.13 (m, 1H), 6.72 (s, 1H), 7.03 (d, J=8.80 Hz, 2H), 7.39-7.48 (m, 2H), 7.66-7.00 (m, 1H), 8.03 (d, J=8.80 Hz, 2H), 8.59 (s, 1H), 8.92 (s, 1H), 10.03 (s, 1H), 11.09 (s, 1H). Mass spec m/z 804.7 [M+H]⁺.

### Step 3

### Example 83: 4-(2-(1-(2-(2,6-dioxopiperidin-3-yl)-1,3-dioxoisoindolin-5-yl)piperidin-4-yl)ethoxy)-N-(2-((R)-1-methylpyrrolidin-2-yl)-1H-pyrrolo[3,2-c]pyridin-6-yl)benzamide

To a solution of tert-butyl tert-butyl 6-(4-(2-(1-(2-(2,6-dioxopiperidin-3-yl)-1,3-dioxoisoindolin-5-yl)piperidin-4-yl)ethoxy)benzamido)-2-((R)-1-methylpyrrolidin-2-yl)-1H-pyrrolo[3,2-c]pyridine-1-carboxylate (Intermediate 367, 0.4 g, 0.497 mmol) in 1,4-dioxane (4 mL) was added 4M hydrochloric acid in 1,4-dioxane (4 mL) and the reaction mixture was stirred at room temperature for 4 h. The reaction mixture was concentrated in vacuo and the crude material purified by preparative HPLC (Method A) to afford 4-(2-(1-(2-(2,6-dioxopiperidin-3-yl)-1,3-dioxoisoindolin-5-yl)piperidin-4-yl)ethoxy)-N-(2-((R)-1-methylpyrrolidin-2-yl)-1H-pyrrolo[3,2-c]pyridin-6-yl)benzamide (Example 83, 55 mg, 16%) as a yellow solid. ¹H NMR (400 MHz, DMSO-d₆) δ ppm 1.43-1.50 (m, 2H), 1.69-1.83 (m, 8H), 2.00-2.71 (m, 1H), 2.12-2.18 (m, 4H), 2.21-2.28 (m, 1H), 2.52-2.61 (m, 3H), 2.84-2.94 (m, 3H), 3.14-3.17 (m, 1H), 3.69-3.73 (m, 2H), 4.14-4.18 (m, 2H), 5.07-5.11 (m, 1H), 6.38 (s, 1H), 7.04 (d, J=8.80 Hz, 2H), 7.31-7.36 (m, 2H), 7.66-7.70 (m, 1H), 8.04 (d, J=8.80 Hz, 2H), 8.17 (s, 1H), 8.49 (s, 1H), 10.28 (s, 1H), 11.09 (s, 1H), 11.32 (s, 1H). Mass spec m/z 704.2 [M+H]⁺.

### Example 84 was Prepared Following Scheme 83

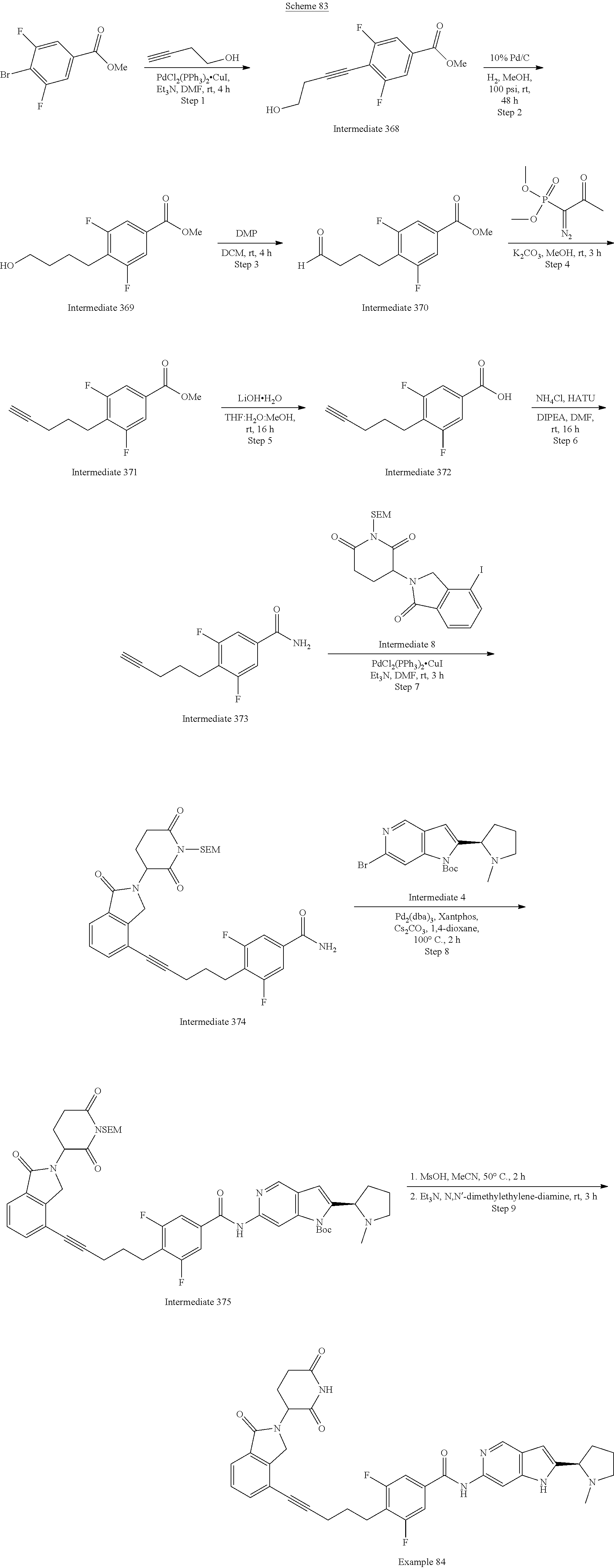

### Step 1

### Intermediate 368: methyl-3,5-difluoro-4-(4-hydroxybut-1-yn-1-yl)benzoate

To a solution of methyl-4-bromo-3,5-difluorobenzoate (CAS No. 1803565-64-1, 10.0 g, 39.8 mmol) in dimethylformamide (30 mL) was added but-3-yn-1-ol (CAS No. 927-74-2, 2.80 g, 39.8 mmol) followed by triethylamine (146.0 g, 143 mmol). The reaction mixture was purged with argon for 15 min then copper (I) iodide (0.8 g, 3.98 mmol) and PdCl₂(PPh₃)₂ (2.82 g, 3.98 mmol) were added. The reaction mixture was purged with argon for a further 10 min and stirred at room temperature for 4 h. The reaction mixture was then diluted with water (300 mL) and extracted with ethyl acetate (3×100 mL). The combined organic layers were dried over anhydrous Na₂SO₄ and concentrated in vacuo. The crude material was purified by combi-flash chromatography, by eluting with 2% MeOH in DCM, to afford methyl-3,5-difluoro-4-(4-hydroxybut-1-yn-1-yl)benzoate (Intermediate 368, 9.0 g, 90%) as a yellow liquid. ¹H NMR (400 MHz, DMSO-d₆) δ ppm 2.68 (t, J=7.60 Hz, 2H), 3.58-3.64 (m, 2H), 3.87 (s, 3H), 4.98 (t, J=5.20 Hz, 1H), 7.61 (d, J=6.80 Hz, 2H). Mass spec m/z 241.1 [M+H]⁺.

### Step 2

### Intermediate 369: methyl-3,5-difluoro-4-(4-hydroxybutyl)benzoate

To a solution of methyl-3,5-difluoro-4-(4-hydroxybut-1-yn-1-yl)benzoate (Intermediate 368, 9.0 g, 37.45 mmol) in MeOH (150 mL) was added 10% Pd/C (3.50 g). The mixture was stirred at room temperature under an atmosphere of hydrogen at 100 psi for 48 h. The reaction mixture was then filtered through a celite bed and the filter pad was washed with MeOH (500 mL). The filtrate was concentrated in vacuo to afford methyl-3,5-difluoro-4-(4-hydroxybutyl)benzoate (Intermediate 369, 9.0 g, 98%) as a yellow solid, which was used for the next step without further purification. ¹H NMR (400 MHz, DMSO-d₆) δ ppm 1.37-1.47 (m, 2H), 1.52-1.62 (m, 2H), 2.68 (t, J=7.20 Hz, 2H), 3.33-3.47 (m, 2H), 3.87 (s, 3H), 4.41 (t, J=5.20 Hz, 1H), 7.51-7.60 (m, 2H).

### Step 3

### Intermediate 370: methyl-3,5-difluoro-4-(4-oxobutyl)benzoate

To a cooled (0° C.) solution of methyl-3,5-difluoro-4-(4-hydroxybutyl)benzoate (Intermediate 369, 9.0 g, 36.85 mmol) in dichloroethane (300 mL) was added Dess-Martin periodinane (CAS No. 87413-09-0, 24.17 g, 55.28 mmol). The reaction mixture was stirred at room temperature for 4 h, diluted with saturated aqueous sodium bicarbonate solution (150 mL) and saturated aqueous sodium thiosulphate solution (150 mL), then extracted with dichloromethane (300 mL). The organic layer was separated, dried over anhydrous Na₂SO₄, filtered and concentrated in vacuo to afford methyl-3,5-difluoro-4-(4-oxobutyl)benzoate (Intermediate 370, 6.70 g, 75%) as a yellow liquid, which was used for the next step without further purification. ¹H NMR (400 MHz, DMSO-d₆) δ ppm 1.74-1.82 (m, 2H), 2.46-2.49 (m, 2H), 2.70 (t, J=7.60 Hz, 2H), 3.87 (s, 3H), 7.59 (d, J=7.60 Hz, 2H), 9.66 (s, 1H). Mass spec m/z 242.4 [M+H]⁺.

### Step 4

### Intermediate 371: methyl-3,5-difluoro-4-(pent-4-yn-1-yl)benzoate

To a solution of methyl-3,5-difluoro-4-(4-oxobutyl)benzoate (Intermediate 370, 6.7 g, 28 mmol) in methanol (80 mL) was added potassium carbonate (7.60 g, 55 mmol) followed by dimethyl (1-diazo-2-oxopropyl)phosphonate (CAS No. 90965-06-3, 8.0 g, 41.0 mmol). The reaction mixture was stirred at room temperature for 3 h. After completion, the reaction mixture was diluted with water (300 mL) and extracted with ethyl acetate (2×300 mL). The combined organic layers were dried over Na₂SO₄ and concentrated in vacuo. The crude material was purified by combi-flash chromatography, by eluting with 5-10% ethyl acetate in heptane, to afford methyl-3,5-difluoro-4-(pent-4-yn-1-yl)benzoate (Intermediate 371, 2.20 g, 33%) as a yellow liquid. ¹H NMR (400 MHz, DMSO-d₆) δ ppm 1.77-1.81 (m, 2H), 2.48 (s, 1H), 2.50-2.52 (m, 2H), 2.67-2.71 (m, 2H), 3.86 (s, 3H), 7.57 (d, J=7.45 Hz, 2H).

### Step 5

### Intermediate 372: 3,5-difluoro-4-(pent-4-yn-1-yl)benzoic acid

To a solution of methyl-3,5-difluoro-4-(pent-4-yn-1-yl)benzoate (Intermediate 371, 2.20 g, 9.20 mmol) in tetrahydrofuran (15.0 mL), water (0.7 mL) and methanol (15.0 mL) was added lithium hydroxide (0.9 g, 37.0 mmol) and the reaction mixture was stirred at room temperature for 16 h. After completion, the reaction mixture was concentrated in vacuo. The obtained residue was diluted with water (10 mL) and acidified to pH~4 by the addition of 1N aqueous HCl solution (50 mL). The resultant precipitate was collected by filtration and dried in vacuo to afford 3,5-difluoro-4-(pent-4-yn-1-yl)benzoic acid (Intermediate 372, 2.0 g, 97%) as an off-white solid which was used for the next step without further purification. ¹H NMR (400 MHz, DMSO-d₆) δ ppm 1.64-1.75 (m, 2H), 2.20-2.25 (m, 2H), 2.76 (t, J=7.60 Hz, 2H), 2.82 (s, 1H), 7.52 (d, J=7.60 Hz, 2H), 13.48 (br s, 1H). Mass spec m/z 223.3 [M−H]⁻.

### Step 6

### Intermediate 373: 3,5-difluoro-4-(pent-4-yn-1-yl)benzamide

To a cooled (0° C.) solution of 3,5-difluoro-4-(pent-4-yn-1-yl)benzoic acid (Intermediate 372, 2.0 g, 8.90 mmol) in dimethylformamide (20 mL) was added HATU (5.36 g, 13.38 mmol) and N,N-diisopropylethylamine (3.52 mL, 26.72 mmol). Ammonium chloride (2.38 g, 44.60 mmol) was then added, and the reaction mixture was stirred at room temperature for 16 h. The reaction mixture was then poured into ice cold water (150 mL) and the mixture stirred for 10 min. The resultant precipitate was collected by filtration and dried in vacuo to afford 3,5-difluoro-4-(pent-4-yn-1-yl)benzamide (Intermediate 373, 1.70 g, 85%) as an off-white solid. ¹H NMR (400 MHz, DMSO-d₆) δ ppm 1.65-1.75 (m, 2H), 2.20-2.24 (m, 2H), 2.75 (t, J=7.60 Hz, 2H), 2.83 (t, J=2.40 Hz, 1H), 7.54-7.56 (m, 2H), 7.62 (br s, 1H), 8.09 (br s, 1H). Mass spec m/z 224.3 [M+H]⁺.

### Step 7

### Intermediate 374: 4-(5-(2-(2,6-dioxo-1-((2-(trimethylsilyl) ethoxy)methyl)piperidin-3-yl)-1-oxoisoindolin-4-yl)pent-4-yn-1-yl)-3,5-difluorobenzamide

To a solution of 3,5-difluoro-4-(pent-4-yn-1-yl)benzamide (Intermediate 373, 0.25 g, 1.12 mmol) in dimethylformamide (5.0 mL) was added 3-(4-iodo-1-oxoisoindolin-2-yl)-1-((2-(trimethylsilyl)ethoxy)methyl)piperidine-2,6-dione (Intermediate 8, 0.56 g, 1.12 mmol) followed by triethylamine (4.10 g, 40.32 mmol). The reaction mixture was purged with argon for 15 min then copper (I) iodide (0.02 g, 0.11 mmol) and PdCl₂(PPh₃)₂ (0.10 g, 0.11 mmol) were added. The reaction mixture was further purged with argon for 10 min and stirred at room temperature for 3 h. After completion, the reaction mixture was diluted with water (200 mL) and extracted with ethyl acetate (3×100 mL). The combined organic layers were dried over anhydrous Na₂SO₄ and concentrated in vacuo. The crude material was purified by combi-flash chromatography, by eluting with 28% ethyl acetate in heptane, to afford 4-(5-(2-(2,6-dioxo-1-((2-(trimethylsilyl)ethoxy)methyl)piperidin-3-yl)-1-oxoisoindolin-4-yl)pent-4-yn-1-yl)-3,5-difluorobenzamide (Intermediate 374, 0.5 g, 75%) as a yellow solid. ¹H NMR (400 MHz, DMSO-d₆) δ ppm −0.06 (s, 9H), 0.75-0.85 (m, 2H), 1.80-1.90 (m, 2H), 2.04-2.10 (m, 1H), 2.31-2.44 (m, 2H), 2.54-2.56 (m, 1H), 2.77-2.85 (m, 3H), 3.02-3.12 (m, 1H), 3.46-3.52 (m, 2H), 4.23-4.30 (m, 1H), 4.44-4.52 (m, 1H), 5.00-5.10 (m, 2H), 5.28-5.32 (m, 1H), 7.52-7.58 (m, 2H), 7.60-7.62 (m, 2H), 7.64 (br s, 1H), 7.73 (d, J=7.20 Hz, 1H), 8.07 (br s, 1H). Mass spec m/z 593.9 [M−H]⁻.

### Step 8

### Intermediate 375: tert-butyl-6-(4-(5-(2-(2,6-dioxo-1-((2-(trimethylsilyl)ethoxy)methyl)piperidin-3-yl)-1-oxoisoindolin-4-yl)pent-4-yn-1-yl)-3,5-difluorobenzamido)-2-((R)-1-methylpyrrolidin-2-yl)-1H-pyrrolo[3,2-c]pyridine-1-carboxylate

To a solution of 4-(5-(2-(2,6-dioxo-1-((2-(trimethylsilyl)ethoxy)methyl)piperidin-3-yl)-1-oxoisoindolin-4-yl) pent-4-yn-1-yl)-3,5-difluorobenzamide (Intermediate 374, 0.5 g, 0.56 mmol) in 1,4-dioxane (10 mL) was added tert-butyl-(R)-6-bromo-2-(1-methylpyrrolidin-2-yl)-1H-pyrrolo[3,2-c]pyridine-1-carboxylate (Intermediate 4, 0.350 g, 0.92 mmol) followed by cesium carbonate (0.9 g, 2.76 mmol). The reaction mixture was purged with argon for 15 min, then Pd₂(dba)₃ (0.13 g, 0.14 mmol) followed by Xantphos (0.16 g, 0.28 mmol) were added. The reaction mixture was further purged with argon for 10 min and heated at 100° C. for 2 h. After completion, the reaction mixture was concentrated in vacuo to obtain the crude compound. The crude material was purified by combi-flash chromatography, by eluting with 90% ethyl acetate in heptane, to afford tert-butyl-6-(4-(5-(2-(2,6-dioxo-1-((2-(trimethylsilyl)ethoxy)methyl)piperidin-3-yl)-1-oxoisoindolin-4-yl)pent-4-yn-1-yl)-3,5-difluorobenzamido)-2-((R)-1-methylpyrrolidin-2-yl)-1H-pyrrolo[3,2-c]pyridine-1-carboxylate (Intermediate 375, 0.5 g, 61%) as a yellow solid. ¹H NMR (400 MHz, DMSO-d₆) δ ppm −0.06 (s, 9H), 0.72-0.83 (m, 2H), 1.69 (s, 9H), 1.72-1.76 (m, 4H), 1.82-1.87 (m, 2H), 2.02-2.07 (m, 1H), 2.35 (s, 3H), 2.39-2.45 (m, 2H), 2.56 (t, J=7.60 Hz, 2H), 2.75-2.88 (m, 3H), 3.02-3.09 (m, 2H), 3.35-3.54 (m, 2H), 3.83-3.95 (m, 1H), 4.24-4.30 (m, 1H), 4.44-4.52 (m, 1H), 5.01-5.09 (m, 2H), 5.23-5.30 (m, 1H), 6.76 (s, 1H), 7.54 (t, J=7.60 Hz, 1H), 7.60 (d, J=7.60 Hz, 1H), 7.71-7.80 (m, 3H), 8.61 (s, 1H), 8.89 (s, 1H), 10.88 (s, 1H). Mass spec m/z 894.6 [M+H]⁺.

### Step 9

### Example 84: 4-(5-(2-(2,6-dioxopiperidin-3-yl)-1-oxoisoindolin-4-yl)pent-4-yn-1-yl)-3,5-difluoro-N-(2-((R)-1-methylpyrrolidin-2-yl)-1H-pyrrolo[3,2-c]pyridin-6-yl benzamide

To a solution of tert-butyl-6-(4-(5-(2-(2,6-dioxo-1-((2-(trimethylsilyl)ethoxy)methyl)piperidin-3-yl)-1-oxoisoindolin-4-yl)pent-4-yn-1-yl)-3,5-difluorobenzamido)-2-((R)-1-methylpyrrolidin-2-yl)-1H-pyrrolo[3,2-c]pyridine-1-carboxylate (Intermediate 375, 0.5 g, 0.56 mmol) in acetonitrile (10 mL) was added methanesulfonic acid (0.55 g, 5.56 mmol) and the reaction mixture was heated at 50° C. for 2 h. After cooling to room temperature, N,N′-dimethylethylenediamine (0.27 g, 2.79 mmol) followed by triethylamine (1.14 g, 11.17 mmol) were added, and the reaction mixture was stirred at room temperature for 3 h. The reaction mixture was then poured into ice cold water (80 mL), the resultant precipitate was collected by filtration and dried in vacuo to afford the crude compound. The crude material was purified by preparative HPLC (Method A) to afford 4-(5-(2-(2,6-dioxopiperidin-3-yl)-1-oxoisoindolin-4-yl)pent-4-yn-1-yl)-3,5-difluoro-N-(2-((R)-1-methylpyrrolidin-2-yl)-1H-pyrrolo[3,2-c]pyridin-6-yl)benzamide (Example 84, 0.13 g, 35%) as an off-white solid. ¹H NMR (400 MHz, DMSO-d₆) δ ppm 1.84-1.92 (m, 5H), 2.02-2.04 (m, 1H), 2.12-2.216 (m, 1H), 2.17 (s, 3H), 2.25-2.30 (m, 1H), 2.38-2.42 (m, 1H), 2.52-2.61 (m, 3H), 2.83-2.92 (m, 3H), 3.12-3.17 (m, 1H), 3.30-3.34 (m, 1H), 4.30-4.36 (m, 1H), 4.46-4.50 (m, 1H), 5.12-5.16 (m, 1H), 6.39 (s, 1H), 7.53 (t, J=7.60 Hz, 1H), 7.62 (d, J=6.80 Hz, 1H), 7.71 (d, J=6.80 Hz, 1H), 7.78 (d, J=7.60 Hz, 2H), 8.19 (s, 1H), 8.52 (s, 1H), 10.65 (br s, 1H), 11.02 (br s, 1H), 11.41 (s, 1H). Mass spec m/z 663.3 [M−H]⁻.

### Example 85 was Prepared Following Scheme 84

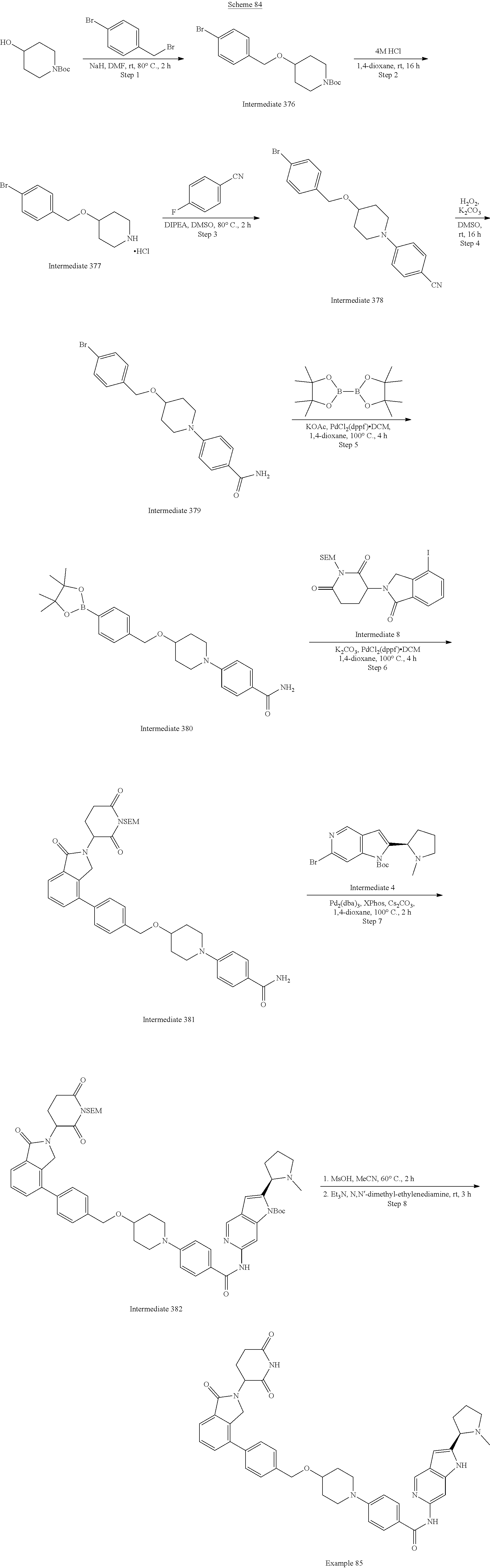

### Step 1

### Intermediate 376: tert-butyl 4-((4-bromobenzyl)oxy)piperidine-1-carboxylate

To a solution of tert-butyl 4-hydroxypiperidine-1-carboxylate (CAS No. 109384-19-2, 10.0 g, 49.68 mmol) and 1-bromo-4-(bromomethyl)benzene (CAS No. 589-15-1, 14.90 g, 59.62 mmol) in dimethylformamide (200 mL) was added sodium hydride (13.91 g, 347.81 mmol) and the reaction mixture was heated at 80° C. for 12 h. After completion, the reaction mixture was quenched with water (500 mL) and extracted with dichloromethane (2×250 mL). The combined organic layers were dried over anhydrous Na₂SO₄, filtered and concentrated in vacuo. The crude material was purified by combi-flash chromatography, by eluting with 60% ethyl acetate in heptane, to afford tert-butyl 4-((4-bromobenzyl)oxy)piperidine-1-carboxylate (Intermediate 376, 14.2 g, 77%) as a white solid. ¹H NMR (400 MHz, DMSO-d₆) δ ppm 1.34-1.36 (m, 1H), 1.39 (s, 9H), 1.41-1.44 (m, 1H), 1.79-1.83 (m, 2H), 3.03 (t, J=9.74 Hz, 2H), 3.52-3.64 (m, 3H), 4.49 (s, 2H), 7.29 (d, J=8.29 Hz, 2H), 7.53 (d, J=8.29 Hz, 2H). Mass spec m/z 314.0 [M+H−56]⁺.

### Step 2

### Intermediate 377: 4-((4-bromobenzyl)oxy)piperidine hydrochloride

To a cooled (0° C.) solution of tert-butyl 4-((4-bromobenzyl)oxy)piperidine-1-carboxylate (Intermediate 376, 12.0 g, 32.41 mmol) in 1,4-dioxane (50 mL) was added 4M hydrochloric acid in 1,4-dioxane (100 mL) and the reaction mixture was stirred at room temperature for 16 h. After completion, the reaction mixture was concentrated in vacuo to afford 4-((4-bromobenzyl)oxy)piperidine hydrochloride (Intermediate 377, 8.80 g, 88%) as an off-white solid, which was used for the next step without further purification. ¹H NMR (400 MHz, DMSO-d₆) δ ppm 1.70-1.78 (m, 2H), 1.96-2.01 (m, 2H), 2.90-3.00 (m, 2H), 3.09-3.13 (m, 2H), 3.63-3.67 (m, 1H), 4.49 (s, 2H), 7.31 (d, J=8.22 Hz, 2H), 7.55 (d, J=8.22 Hz, 2H), 8.95 (br s, 2H). Mass spec m/z 272.0 [M+H+2]⁺.

### Step 3

### Intermediate 378: 4-(4-((4-bromobenzyl)oxy)piperidin-1-yl)benzonitrile

To a solution of 4-((4-bromobenzyl)oxy)piperidine hydrochloride (Intermediate 377, 5.00 g, 16.30 mmol) and 4-fluorobenzonitrile (CAS No. 1194-02-1, 3.94 g, 32.61 mmol) in dimethyl sulfoxide (100 mL) was added N,N-diisopropylethylamine (12.9 g, 97.83 mmol). The reaction mixture was heated at 80° C. for 12 h. After completion, the reaction mixture was quenched with water (250 mL) and extracted with dichloromethane (250 mL). The organic layer was separated, washed with water (150 mL), dried over anhydrous Na₂SO₄, filtered and concentrated in vacuo. The crude material was purified by combi-flash chromatography, by eluting with 50% ethyl acetate in heptane, to afford 4-(4-((4-bromobenzyl)oxy)piperidin-1-yl)benzonitrile (Intermediate 378, 5.0 g, 86%) as an off-white solid. ¹H NMR (400 MHz, DMSO-d₆) δ ppm 1.44-1.56 (m, 2H), 1.82-1.94 (m, 2H), 3.04-3.15 (m, 2H), 3.56-3.70 (m, 3H), 4.49 (s, 2H), 6.98 (d, J=9.12 Hz, 2H), 7.27 (d, J=8.29 Hz, 2H), 7.48-7.52 (m, 4H). Mass spec m/z 371.0 [M+H]⁺.

### Step 4

### Intermediate 379: 4-(4-((4-bromobenzyl)oxy)piperidin-1-yl)benzamide

To a solution of 4-(4-((4-bromobenzyl)oxy)piperidin-1-yl)benzonitrile (Intermediate 378, 3.00 g, 8.08 mmol) in dimethyl sulfoxide (50 mL) was added potassium carbonate (1.67 g, 12.12 mmol) and hydrogen peroxide (1.26 mL, 16.16 mmol). The reaction mixture was stirred at room temperature for 16 h. After completion, the reaction mixture was diluted with water (200 mL) and the resulting precipitate was filtered and dried in vacuo. The crude material was triturated with diethyl ether (2×30 mL) to afford 4-(4-((4-bromobenzyl)oxy)piperidin-1-yl)benzamide (Intermediate 379, 2.5 g, 79%) as an off-white solid. ¹H NMR (400 MHz, DMSO-d₆) δ ppm 1.48-1.59 (m, 2H), 1.86-1.96 (m, 2H), 2.95-3.05 (m, 2H), 3.56-3.62 (m, 3H), 4.49 (s, 2H), 6.89 (d, J=9.12 Hz, 3H), 7.28 (d, J=8.29 Hz, 2H), 7.50 (d, J=8.29 Hz, 2H), 7.69 (d, J=8.71 Hz, 2H), exchangeable proton not observed. Mass spec m/z 389.0 [M+H]⁺.

### Step 5

### Intermediate 380: 4-(4-((4-(4,4,5,5-tetramethyl-1,3,2-dioxaborolan-2-yl)benzyl)oxy)piperidin-1-yl)benzamide

To a solution of 4-(4-((4-bromobenzyl)oxy)piperidin-1-yl)benzamide (Intermediate 379, 2.00 g, 5.13 mmol) and 4,4,4′,4′,5,5,5′,5′-octamethyl-2,2′-bi(1,3,2-dioxaborolane) (CAS No. 73183-34-3, 1.69 g, 6.67 mmol) in 1,4-dioxane (30 mL) was added potassium acetate (1.51 g, 15.41 mmol). The reaction mixture was purged with argon for 20 min then PdCl₂(dppf) DCM (0.41 g, 0.51 mmol) was added and the reaction mixture was heated at 100° C. for 4 h. After completion, the reaction mixture was concentrated in vacuo. The residue was triturated with diethyl ether (2×20 mL) and dried in vacuo to afford 4-(4-((4-(4,4,5,5-tetramethyl-1,3,2-dioxaborolan-2-yl)benzyl)oxy)piperidin-1-yl)benzamide (Intermediate 380, 3.0 g) as an off grey solid, which was used for the next step without further purification. Mass spec m/z 437.2 [M+H]⁺.

### Step 6

### Intermediate 381: 4-(4-((4-(2-(2,6-dioxo-1-((2-(trimethylsilyl)ethoxy)methyl)piperidin-3-yl)-1-oxoisoindolin-4-yl)benzyl)oxy)piperidin-1-yl)benzamide

To a solution of 4-(4-((4-(4,4,5,5-tetramethyl-1,3,2-dioxaborolan-2-yl)benzyl)oxy)piperidin-1-yl)benzamide (Intermediate 380, 3.5 g, 8.0 mmol) in 1,4-dioxane (40 mL) was added 3-(4-iodo-1-oxoisoindolin-2-yl)-1-((2-(trimethylsilyl)ethoxy)methyl)piperidine-2,6-dione (Intermediate 8, 2.0 g, 4.0 mmol) followed by potassium carbonate (1.7 g, 12.0 mmol). The reaction mixture was purged with argon for 15 min then PdCl₂(dppf) DCM (0.33 g, 0.40 mmol) was added. The reaction mixture was further purged with argon for 10 min and heated at 100° C. for 4 h. After completion, the reaction mixture was concentrated in vacuo. The crude material was purified by combi-flash chromatography, by eluting with 3% MeOH in DCM, to afford 4-(4-((4-(2-(2,6-dioxo-1-((2-(trimethylsilyl)ethoxy)methyl)piperidin-3-yl)-1-oxoisoindolin-4-yl)benzyl)oxy)piperidin-1-yl)benzamide (Intermediate 381, 1.3 g, 48%) as a yellow solid. ¹H NMR (400 MHz, DMSO-d₆) δ ppm −0.10 (s, 9H), 0.75-0.83 (m, 2H), 1.54-1.60 (m, 2H), 1.93-2.01 (m, 2H), 2.30-2.40 (m, 2H), 2.76-3.01 (m, 1H), 3.04-3.08 (m, 3H), 3.44-3.51 (m, 2H), 3.56-3.67 (m, 3H), 4.28-4.35 (m, 1H), 4.58-4.64 (m, 3H), 4.96-5.05 (m, 2H), 5.23-5.27 (m, 1H), 6.89-6.98 (m, 2H), 7.44 (d, J=7.83 Hz, 2H), 7.54 (d, J=7.43 Hz, 3H), 7.59-7.74 (m, 5H). Mass spec m/z 683.3 [M+H]⁺.

### Step 7

### Intermediate 382: tert-butyl 6-(4-(4-((4-(2-(2,6-dioxo-1-((2-(trimethylsilyl) ethoxy)methyl) piperidin-3-yl)-1-oxoisoindolin-4-yl)benzyl)oxy)piperidin-1-yl)benzamido)-2-((R)-1-methylpyrrolidin-2-yl)-1H-pyrrolo[3,2-c]pyridine-1-carboxylate

To a solution of 4-(4-((4-(2-(2,6-dioxo-1-((2-(trimethylsilyl)ethoxy)methyl)piperidin-3-yl)-1-oxoisoindolin-4-yl)benzyl)ox y) piperidin-1-yl)benzamide (Intermediate 381, 1.0 g, 1.0 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.70 g, 2.0 mmol) followed by cesium carbonate (1.0 g, 4.0 mmol). The reaction mixture was purged with argon for 15 min, followed by addition of Pd₂(dba)₃ (0.3 g, 0.3 mmol) and Xantphos (0.3 g, 0.6 mmol). The reaction mixture was further purged with argon for 10 min and heated at 100° C. for 2 h. After completion, the reaction mixture was filtered through a celite bed, the filter pad was washed with ethyl acetate (100 mL) and the filtrate was concentrated in vacuo. The crude material was purified by combi-flash chromatography, by eluting with 5% MeOH in DCM, to afford tert-butyl-6-(4-(4-((4-(2-(2,6-dioxo-1-((2-(trimethylsilyl)ethoxy)methyl)piperidin-3-yl)-1-oxoisoindolin-4-yl)benzyl)oxy)piperidin-1-yl)benzamido)-2-((R)-1-methylpyrrolidin-2-yl)-1H-pyrrolo[3,2-c]pyridine-1-carboxylate (Intermediate 382, 0.75 g, 30%) as a brown solid. ¹H NMR (400 MHz, DMSO-d₆) δ ppm −0.03 (s, 9H), 0.76-0.79 (m, 2H), 1.65 (s, 9H), 1.52-1.63 (m, 2H), 1.69-1.75 (m, 2H), 1.91-2.04 (m, 6H), 2.22-2.29 (m, 2H), 2.31 (s, 3H), 1.91-2.04 (m, 4H), 2.98-3.15 (m, 4H), 3.41-3.52 (m, 3H), 4.28-4.32 (m, 2H), 4.58 (s, 2H), 5.73 (s, 2H), 6.70 (s, 1H), 6.96-7.00 (m, 2H), 7.31-7.77 (m, 7H), 7.91-7.93 (m, 2H), 8.53 (s, 1H), 8.89 (s, 1H), 10.30 (br s, 1H). Mass spec m/z 982.5 [M+H]⁺.

### Step 8

### Example 85: 4-(4-((4-(2-(2,6-dioxopiperidin-3-yl)-1-oxoisoindolin-4-yl)benzyl)oxy)piperidin-1-yl)-N-(2-((R)-1-methylpyrrolidin-2-yl)-1H-pyrrolo[3,2-c]pyridin-6-yl)benzamide

To a solution of tert-butyl 6-(4-(4-((4-(2-(2,6-dioxo-1-((2-(trimethylsilyl)ethoxy)methyl)piperidin-3-yl)-1-oxoisoindolin-4-yl)benzyl)oxy)piperidin-1-yl)benzamido)-2-((R)-1-methylpyrrolidin-2-yl)-1H-pyrrolo[3,2-c]pyridine-1-carboxylate (Intermediate 382, 0.75 g, 0.76 mmol) in acetonitrile (20 mL) was added methanesulfonic acid (0.50 mL, 7.63 mmol) and the reaction mixture was heated at 50° C. for 2 h. After cooling to room temperature, N,N′-dimethylethylenediamine (0.45 mL, 3.81 mmol) followed by triethylamine (2.14 mL, 15.27 mmol) were added, and the reaction mixture was stirred at room temperature for 3 h. After completion, the reaction mixture was concentrated in vacuo, the residue was diluted with water (120 mL) and the resulting precipitate was filtered and dried in vacuo to obtain the crude compound. The crude material was purified by preparative HPLC (Method A) to afford 4-(4-((4-(2-(2,6-dioxopiperidin-3-yl)-1-oxoisoindolin-4-yl)benzyl)oxy)piperidin-1-yl)-N-(2-((R)-1-methylpyrrolidin-2-yl)-1H-pyrrolo[3,2-c]pyridin-6-yl)benzamide (Example 85, 0.05 g, 10%) as an off-white solid. ¹H NMR (400 MHz, DMSO-d₆) δ ppm 1.56-1.65 (m, 2H), 1.74-1.95 (m, 4H), 1.95-2.05 (m, 3H), 2.09-2.14 (m, 1H), 2.16 (s, 3H), 2.21-2.29 (m, 1H), 2.39-2.45 (m, 1H), 2.54-2.61 (m, 1H), 2.85-2.96 (m, 1H), 3.08-3.18 (m, 3H), 3.68-3.71 (m, 3H), 4.39-4.44 (m, 1H), 4.61-4.65 (m, 3H), 5.11-5.16 (m, 1H), 6.37 (s, 1H), 7.00 (d, J=8.88 Hz, 2H), 7.48 (d, J=8.13 Hz, 2H), 7.56-7.61 (m, 2H), 7.61-7.67 (m, 1H), 7.68-7.73 (m, 1H), 7.76 (d, J=7.38 Hz, 1H), 7.95 (d, J=8.88 Hz, 2H), 8.17 (s, 1H), 8.47 (s, 1H), 10.06 (br s, 1H), 10.96 (br s, 1H), 11.31 (br s, 1H). Mass spec m/z 752.0 [M+H]⁺.

### Example 86 was Prepared Following Scheme 85

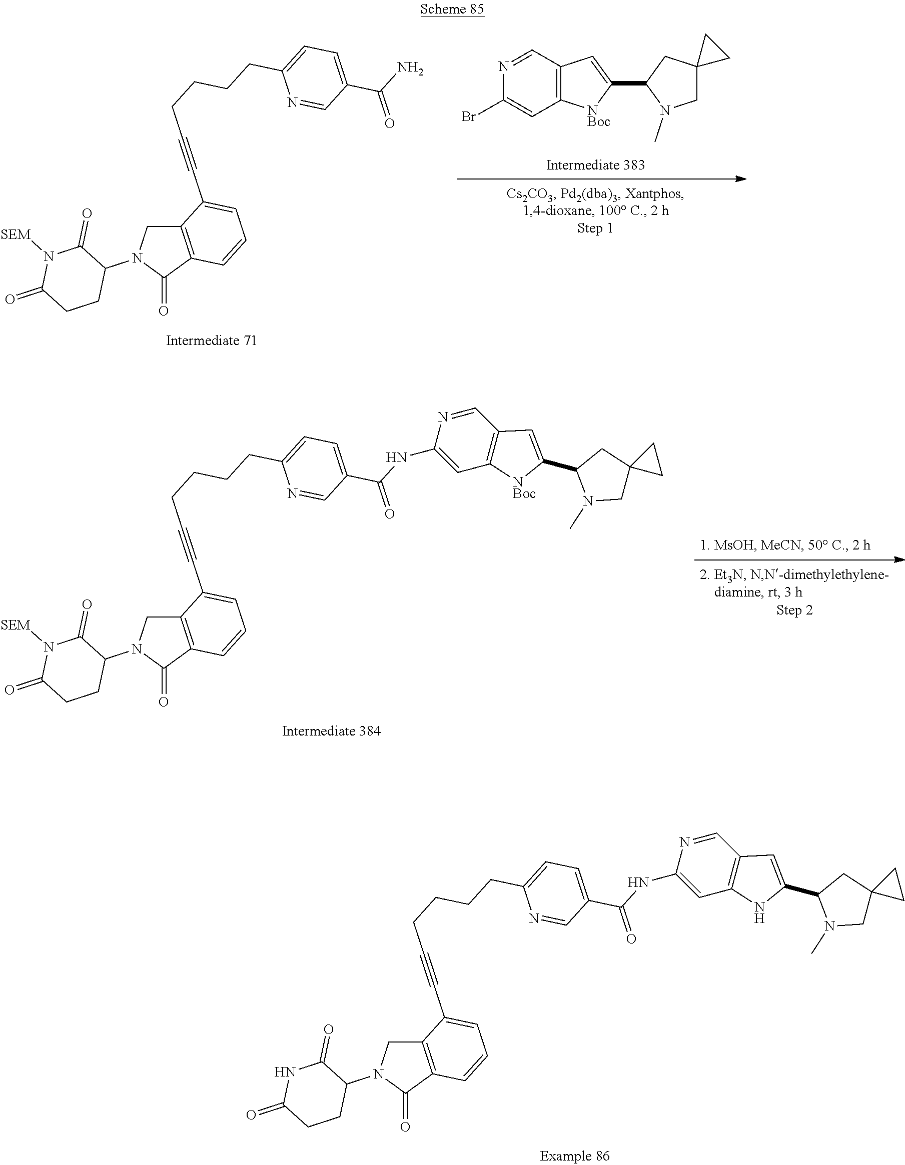

### Step 1

### Intermediate 384: tert-butyl-6-(6-(6-(2-(2,6-dioxo-1-((2-(trimethylsilyl)ethoxy)methyl)piperidin-3-yl)-1-oxoisoindolin-4-yl)hex-5-yn-1-yl)nicotinamido)-2-((rel-R)-5-methyl-5-azaspiro[2.4]heptan-6-yl)-1Hpyrrolo[3,2-c]pyridine-1-carboxylate

To a solution of 6-(6-(2-(2,6-dioxo-1-((2-(trimethylsilyl)ethoxy)methyl)piperidin-3-yl)-1-oxoisoindolin-4-yl)hex-5-yn-1-yl)nicotinamide (Intermediate 71, 0.27 g, 0.47 mmol) in 1,4-dioxane (15 mL) was added tert-butyl-(rel-R)-6-bromo-2-(5-methyl-5-azaspiro[2.4]heptan-6-yl)-1H-pyrrolo[3,2-c]pyridine-1-carboxylate (Intermediate 383, 0.23 g, 0.57 mmol) followed by cesium carbonate (0.46 g, 1.43 mmol). The reaction mixture was purged with argon for 15 min, then Pd₂(dba)₃ (0.06 g, 0.07 mmol) and Xantphos (0.08 g, 0.14 mmol) were added. The reaction mixture was further purged with argon for 10 min and heated at 100° C. for 2 h. The reaction mixture was filtered through a celite bed, the filter pad was washed with ethyl acetate (100 mL) and the filtrate concentrated in vacuo. The obtained residue was triturated with diethyl ether (2×50 mL) and dried in vacuo. The crude material was purified by combi-flash chromatography, by eluting with 4% MeOH in DCM, to afford tert-butyl-6-(6-(6-(2-(2,6-dioxo-1-((2-(trimethylsilyl)ethoxy)methyl)piperidin-3-yl)-1-oxoisoindolin-4-yl)hex-5-yn-1-yl)nicotinamido)-2-((rel-R)-5-methyl-5-azaspiro[2.4]heptan-6-yl)-1H-pyrrolo[3,2-c]pyridine-1-carboxylate (Intermediate 384, 0.23 g, 53%) as a pale-yellow solid. ¹H NMR (400 MHz, DMSO-d₆) δ ppm −0.08 (s, 9H), 0.32-0.41 (m, 1H), 0.43-0.63 (m, 3H), 0.71-0.85 (m, 2H), 1.54-1.63 (m, 3H), 1.66 (s, 9H), 1.68-1.72 (m, 1H), 1.77-1.87 (m, 2H), 1.96-2.09 (m, 1H), 2.23-2.39 (m, 4H), 2.56-2.67 (m, 2H), 2.68-2.91 (m, 4H), 2.96-3.09 (m, 1H), 3.11-3.16 (m, 1H), 3.44-3.49 (m, 2H), 4.10-4.18 (m, 1H), 4.19-4.31 (m, 1H), 4.41-4.51 (m, 1H), 4.96-5.04 (m, 2H), 5.20-5.25 (m, 1H), 6.81 (s, 1H), 7.38 (d, J=8.04 Hz, 1H), 7.45-7.55 (m, 1H), 7.61-7.72 (m, 2H), 8.20-8.29 (m, 1H), 8.58 (s, 1H), 8.89 (s, 1H), 9.03 (s, 1H), 10.89 (br s, 1H). Mass spec m/z 900.5 [M+H]⁺.

### Step 2

### Example 86: 6-(6-(2-(2,6-dioxopiperidin-3-yl)-1-oxoisoindolin-4-yl)hex-5-yn-1-yl)-N-(2-((rel-R)-5-methyl-5-azaspiro[2.4]heptan-6-yl)-1H-pyrrolo[3,2-c]pyridin-6-yl)nicotinamide

To a solution of tert-butyl-6-(6-(6-(2-(2,6-dioxo-1-((2-(trimethylsilyl)ethoxy)methyl)piperidin-3-yl)-1-oxoisoindolin-4-yl)hex-5-yn-1-yl)nicotinamido)-2-((rel-R)-5-methyl-5-azaspiro[2.4]heptan-6-yl)-1H-pyrrolo[3,2-c]pyridine-1-carboxylate (Intermediate 384, 0.20 g, 0.22 mmol) in acetonitrile (6 mL) was added methanesulfonic acid (0.14 mL, 2.22 mmol) and the reaction mixture was heated at 50° C. for 2 h. The reaction mixture was cooled to room temperature, then N,N′-dimethylethylenediamine (0.26 mL, 2.22 mmol) followed by triethylamine (0.62 mL, 4.44 mmol) were added and the mixture stirred at room temperature for 3 h. The reaction mixture was poured into ice cold water (80 mL) and the resultant precipitate was collected by filtration, washed with n-pentane (2×40 mL) and dried in vacuo to obtain the crude compound. The crude material was purified by preparative HPLC (Method A) to afford 6-(6-(2-(2,6-dioxopiperidin-3-yl)-1-oxoisoindolin-4-yl)hex-5-yn-1-yl)-N-(2-((R)-5-methyl-5-azaspiro[2.4]heptan-6-yl)-1H-pyrrolo[3,2-c]pyridin-6-yl)nicotinamide (Example 86, 0.08 g, 57%) as an off-white solid. ¹H NMR (400 MHz, DMSO-d₆) δ ppm 0.49-0.58 (m, 2H), 0.59-0.72 (m, 2H), 1.60-1.67 (m, 2H), 1.84-1.92 (m, 2H), 1.99-2.10 (m, 3H), 2.16 (s, 3H), 2.39-2.43 (m, 1H), 2.53-2.62 (m, 4H), 2.85-2.91 (m, 4H), 3.58-3.63 (m, 1H), 4.27-4.36 (m, 1H), 4.41-4.49 (m, 1H), 5.11-5.15 (m, 1H), 6.43 (s, 1H), 7.41 (d, J=8.00 Hz, 1H), 7.48-7.55 (m, 1H), 7.64 (d, J=6.88 Hz, 1H), 7.71 (d, J=7.50 Hz, 1H), 8.19 (s, 1H), 8.29 (dd, J=8.07, 2.31 Hz, 1H), 8.51 (s, 1H), 9.07 (s, 1H), 10.67 (br s, 1H), 11.00 (br s, 1H), 11.48 (br s, 1H). Mass spec m/z 670.3 [M+H]⁺.

### Intermediate 383 was Synthesised Following Scheme 86

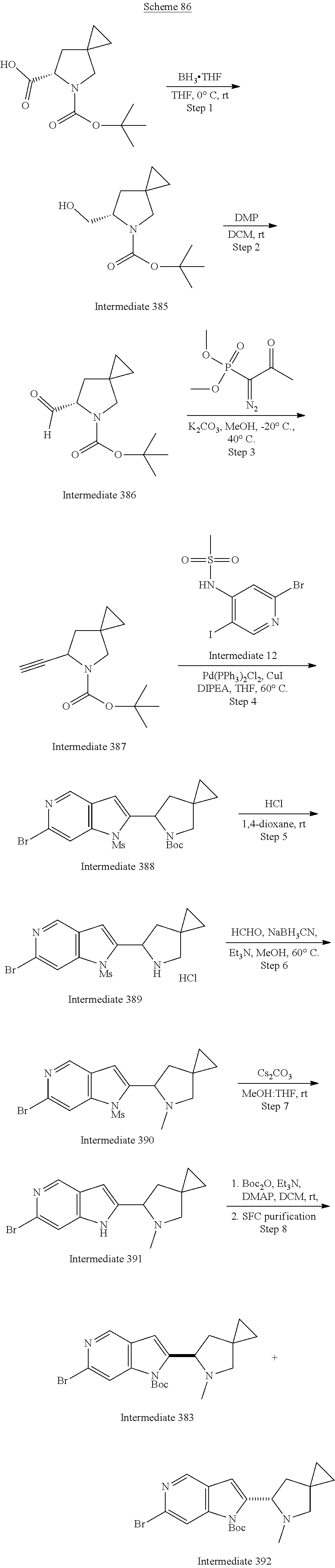

### Step 1

### Intermediate 385: tert-butyl (S)-6-(hydroxymethyl)-5-azaspiro [2.4]heptane-5-carboxylate

To a cooled (0° C.) solution of (S)-5-(tert-butoxycarbonyl)-5-azaspiro[2.4]heptane-6-carboxylic acid (CAS No: 1129634-44-1, 10.0 g, 41.4 mmol) in tetrahydrofuran (150 mL) was added borane-tetrahydrofuran complex (62.2 mL, 62.2 mmol) in tetrahydrofuran (150 mL) and the reaction mixture was stirred at room temperature for 4 h. The reaction was quenched with MeOH (25 mL) and concentrated in vacuo to afford tert-butyl (S)-6-(hydroxymethyl)-5-azaspiro[2.4]heptane-5-carboxylate (Intermediate 385, 9.0 g, 96%) as a brown liquid, which was used for the next step without further purification. ¹H NMR (400 MHz, DMSO-d₆) δ ppm 0.44-0.63 (m, 4H), 1.39 (s, 9H), 1.57-1.72 (m, 1H), 1.94-2.05 (m, 1H), 2.88-3.00 (m, 1H), 3.33-3.43 (m, 1H), 3.50-3.61 (m, 1H), 3.80-3.86 (m, 1H), 4.26-4.40 (m, 1H), 4.70 (br s, 1H). Mass spec m/z 172.5 [M−56]+.

### Step 2

### Intermediate 386: tert-butyl (S)-6-formyl-5-azaspiro [2.4]heptane-5-carboxylate

To a cooled (0° C.) solution of tert-butyl (S)-6-(hydroxymethyl)-5-azaspiro[2.4]heptane-5-carboxylate (Intermediate 385, 10.0 g, 43.99 mmol) in dichloromethane (150 mL) was added Dess-Martin periodinane (28.85 g, 65.99 mmol) and the reaction was stirred at room temperature for 4 h. The reaction mixture was diluted with water (500 mL) and extracted with dichloromethane (3×200 mL). The combined organic layers were dried over anhydrous Na₂SO₄, filtered, and concentrated in vacuo. The crude material was purified by combi-flash chromatography, by eluting with 20% ethyl acetate in heptane, to afford tert-butyl (S)-6-formyl-5-azaspiro[2.4]heptane-5-carboxylate (Intermediate 386, 6.9 g, 70%) as a yellow liquid. ¹H NMR (400 MHz, DMSO-d₆) δ ppm 0.40-0.48 (m, 1H), 0.51-0.64 (m, 3H), 1.41 (s, 9H), 1.75-1.87 (m, 1H), 2.03-2.14 (m, 1H), 3.19-3.26 (m, 1H), 3.28-3.36 (m, 1H), 4.16-4.25 (m, 1H), 9.48-9.54 (m, 1H).

### Step 3

### Intermediate 387: tert-butyl-6-ethynyl-5-azaspiro [2.4]heptane-5-carboxylate

To a cooled (−20° C.) solution of tert-butyl (S)-6-formyl-5-azaspiro [2.4]heptane-5-carboxylate (Intermediate 386, 6.40 g, 28.0 mmol) in methanol (70 mL) was added potassium carbonate (4.7 g, 34.0 mmol) followed by dimethyl (1-diazo-2-oxopropyl)phosphonate (CAS No: 90965-06-3, 8.2 g, 43.0 mmol). The reaction mixture was stirred at 40° C. for 3 h. The reaction mixture was diluted with water (500 mL) and extracted with dichloromethane (3×100 mL). The combined organic layers were dried over anhydrous Na₂SO₄, filtered, and concentrated in vacuo. The crude material was purified by combi-flash chromatography, by eluting with 5% ethyl acetate in heptane, to afford tert-butyl-6-ethynyl-5-azaspiro [2.4]heptane-5-carboxylate (Intermediate 387, 5.20 g, 75%) as a yellow solid. ¹H NMR (400 MHz, DMSO-d₆) δ ppm 0.50-0.70 (m, 4H), 1.41 (s, 9H), 1.50-1.71 (m, 1H), 2.27-2.32 (m, 1H), 2.98-3.17 (m, 2H), 3.22-3.37 (m, 1H), 4.40-4.54 (m, 1H).

### Step 4

### Intermediate 388: tert-butyl-6-(6-bromo-1-(methylsulfonyl)-1H-pyrrolo[3,2-c]pyridin-2-yl)-5-azaspiro[2.4]heptane-5-carboxylate

To a solution of N-(2-bromo-5-iodopyridin-4-yl)methanesulfonamide (Intermediate 12, 7.30 g, 19.36 mmol) in tetrahydrofuran (100 mL) was added tert-butyl-6-ethynyl-5-azaspiro[2.4]heptane-5-carboxylate (Intermediate 387, 5.14 g, 23.24 mmol) and N,N-diisopropylethylamine (27.1 mL, 155 mmol). The reaction mixture was purged with argon for 15 min, then copper (I) iodide (0.37 g, 1.93 mmol) and Pd(PPh₃)₂Cl₂ (1.39 g, 1.94 mmol) were added. The reaction mixture was further purged with argon for another 10 min and heated at 60° C. for 4 h. The reaction mixture was diluted with water (100 mL) and extracted with dichloromethane (3×100 mL). The combined organic layers were dried over anhydrous Na₂SO₄, filtered, and concentrated in vacuo. The crude material was purified by combi-flash chromatography, by eluting with 35% ethyl acetate in heptane, to afford tert-butyl-6-(6-bromo-1-(methylsulfonyl)-1H-pyrrolo[3,2-c]pyridin-2-yl)-5-azaspiro[2.4]heptane-5-carboxylate (Intermediate 388, 6.0 g, 66%) as yellow solid. ¹H NMR (401 MHz, DMSO-d₆) δ ppm 0.28-0.52 (m, 2H), 0.53-0.72 (m, 2H), 1.16-1.28 (m, 2H), 1.40 (s, 9H), 3.37 (s, 3H), 3.53-3.66 (m, 2H), 5.30-5.43 (m, 1H), 6.81 (s, 1H), 7.98 (s, 1H), 8.72 (s, 1H).

### Step 5

### Intermediate 389: 6-bromo-1-(methylsulfonyl)-2-(5-azaspiro[2.4]heptan-6-yl)-1H-pyrrolo[3,2-c]pyridine hydrochloride

To a solution of tert-butyl-6-(6-bromo-1-(methylsulfonyl)-1H-pyrrolo[3,2-c]pyridin-2-yl)-5-azaspiro[2.4]heptane-5-carboxylate (Intermediate 388, 6.5 g, 14.0 mmol) in 1,4-dioxane (60 mL) was added 4M HCl in 1,4-dioxane (70 mL). The reaction mixture was stirred at room temperature for 16 h. The reaction mixture was concentrated in vacuo to afford 6-bromo-1-(methylsulfonyl)-2-(5-azaspiro[2.4]heptan-6-yl)-1H-pyrrolo[3,2-c]pyridine hydrochloride (Intermediate 389, 5.0 g, 98%) as a yellow solid, which was used for next step without further purification. ¹H NMR (400 MHz, DMSO-d₆) δ ppm 0.68-0.76 (m, 2H), 0.78-0.92 (m, 2H), 2.26-2.35 (m, 2H), 3.07-3.16 (m, 1H), 3.25-3.35 (m, 1H), 3.82 (s, 3H), 5.35-5.46 (m, 1H), 7.46 (s, 1H), 8.03 (s, 1H), 8.84 (s, 1H), 9.68 (br s, 1H), 9.54 (br s, 1H). Mass spec m/z 370.1 [M+H]⁺.

### Step 6

### Intermediate 390: 6-bromo-2-(5-methyl-5-azaspiro[2.4]heptan-6-yl)-1-(methylsulfonyl)-1H-pyrrolo[3,2-c]pyridine

To a cooled (0° C.) solution of 6-bromo-1-(methylsulfonyl)-2-(5-azaspiro[2.4]heptan-6-yl)-1H-pyrrolo[3,2-c]pyridine hydrochloride (Intermediate 389, 6.5 g, 18.0 mmol) in methanol (70 mL) was added triethylamine (12.4 mL, 88.5 mmol) and formaldehyde (1.95 mL, 52.9 mmol). After stirring for 5 min, sodium cyanoborohydride (2.62 g, 35.4 mmol) was added and the reaction mixture was heated at 60° C. for 4 h. The reaction mixture was diluted with water (500 mL) and extracted with dichloromethane (3×250 mL). The combined organic layers were dried over anhydrous Na₂SO₄, filtered, and concentrated in vacuo. The crude material was purified by combi-flash chromatography, by eluting with 35% ethyl acetate in heptane, to afford 6-bromo-2-(5-methyl-5-azaspiro [2.4]heptan-6-yl)-1-(methylsulfonyl)-1H-pyrrolo[3,2-c]pyridine (Intermediate 390, 4.8 g, 71%) as a yellow solid. ¹H NMR (400 MHz, DMSO-d₆) δ ppm 0.48-0.54 (m, 1H), 0.56-0.58 (m, 2H), 0.59-0.72 (m, 1H), 1.68-1.73 (m, 1H), 2.33 (s, 3H), 2.40-2.46 (m, 1H), 2.60-2.66 (m, 1H), 2.78-2.82 (m, 1H), 3.56 (s, 3H), 4.02-4.08 (m, 1H), 6.99 (s, 1H), 7.96 (s, 1H), 8.70 (s, 1H).

### Step 7

### Intermediate 391: 6-bromo-2-(5-methyl-5-azaspiro[2.4]heptan-6-yl)-1H-pyrrolo[3,2-c]pyridine

To a solution of 6-bromo-2-(5-methyl-5-azaspiro[2.4]heptan-6-yl)-1-(methylsulfonyl)-1H-pyrrolo[3,2-c]pyridine (Intermediate 390, 4.8 g, 12.0 mmol) in methanol (50 mL) and tetrahydrofuran (50 mL) was added cesium carbonate (8.1 g, 25.0 mmol). The reaction mixture was stirred at room temperature for 4 h. The reaction mixture was quenched with water (100 mL) and extracted ethyl acetate (2×200 mL). The combined organic layers were dried over anhydrous Na₂SO₄, filtered, and concentrated in vacuo to afford (6-bromo-2-(5-methyl-5-azaspiro[2.4]heptan-6-yl)-1H-pyrrolo[3,2-c]pyridine (Intermediate 391, 2.8 g, 73%) as a yellow solid, which was used for next step without further purification. ¹H NMR (400 MHz, DMSO-d₆) δ ppm 0.48-0.56 (m, 2H), 0.58-0.72 (m, 2H), 1.94-2.07 (m, 2H), 2.14 (s, 3H), 2.84-2.92 (m, 1H), 3.55-3.59 (m, 1H), 3.62-3.70 (m, 1H), 6.48 (s, 1H), 7.44 (s, 1H), 8.49 (s, 1H), 11.68 (br s, 1H). Mass spec m/z 306.2 [M+H]⁺.

### Step 8

### Intermediate 383: tert-butyl-(rel-R)-6-bromo-2-(5-methyl-5-azaspiro[2.4]heptan-6-yl)-1H-pyrrolo[3,2-c]pyridine-1-carboxylate and Intermediate 392: tert-butyl-(rel-S)-6-bromo-2-(5-methyl-5-azaspiro[2.4]heptan-6-yl)-1H-pyrrolo[3,2-c]pyridine-1-carboxylate

To a cooled (0° C.) solution of 6-bromo-2-(5-methyl-5-azaspiro[2.4]heptan-6-yl)-1H-pyrrolo [3,2-c]pyridine (Intermediate 391, 5.6 g, 18.0 mmol) in dichloromethane (60 mL) was added triethylamine (10.0 mL, 73.0 mmol) and di-tert-butyl dicarbonate (8.1 g, 37.0 mmol) followed by 4-dimethylaminopyridine (0.45 g, 3.7 mmol). The reaction mixture was stirred at room temperature for 4 h. The reaction mixture was diluted with water (300 mL) and extracted with dichloromethane (3×200 mL). The combined organic layers were dried over anhydrous Na₂SO₄, filtered, and concentrated in vacuo. The resulting material was purified by combi-flash chromatography, by eluting with 45% ethyl acetate in heptane to afford crude product. This material was further purified by SFC chiral purification (Column: Chiralpak IK 250*30 mm, 5 μm. Isocratic elution 25% Mobile Phase (A): CO₂, Mobile Phase (B): MeCN+isopropylalcohol) to afford tert-butyl-(rel-S)-6-bromo-2-(5-methyl-5-azaspiro[2.4]heptan-6-yl)-1H-pyrrolo[3,2-c]pyridine-1-carboxylate (Intermediate 383, 1.9 g, 26%) and tert-butyl-(rel-R)-6-bromo-2-(5-methyl-5-azaspiro[2.4]heptan-6-yl)-1H-pyrrolo[3,2-c]pyridine-1-carboxylate (Intermediate 392, 1.40 g, 19%) as a white solid.

Intermediate 383 exhibited:

1st eluting peak, retention time: 7.67 mins.

¹H NMR (400 MHz, DMSO-d₆) δ ppm 0.34-0.42 (m, 1H), 0.46-0.64 (m, 3H), 1.56-1.61 (m, 1H), 1.64 (s, 9H), 2.33 (s, 3H), 2.40-2.47 (m, 1H), 2.61-2.66 (m, 1H), 2.78-2.80 (m, 1H), 4.10-4.16 (m, 1H), 6.89 (s, 1H), 8.04 (s, 1H), 8.63 (s, 1H). Mass spec m/z 408.10 [M+H]⁺.

Intermediate 392 exhibited:

2^{nd} eluting peak, retention time: 9.99 mins.

¹H NMR (400 MHz, DMSO-d₆) δ ppm 0.34-0.42 (m, 1H), 0.46-0.64 (m, 3H), 1.56-1.61 (m, 1H), 1.64 (s, 9H), 2.33 (s, 3H), 2.40-2.47 (m, 1H), 2.61-2.66 (m, 1H), 2.78-2.80 (m, 1H), 4.10-4.16 (m, 1H), 6.89 (s, 1H), 8.04 (s, 1H), 8.63 (s, 1H). Mass spec m/z 408.10 [M+H]⁺.

### Example 87 was Prepared Following Scheme 87

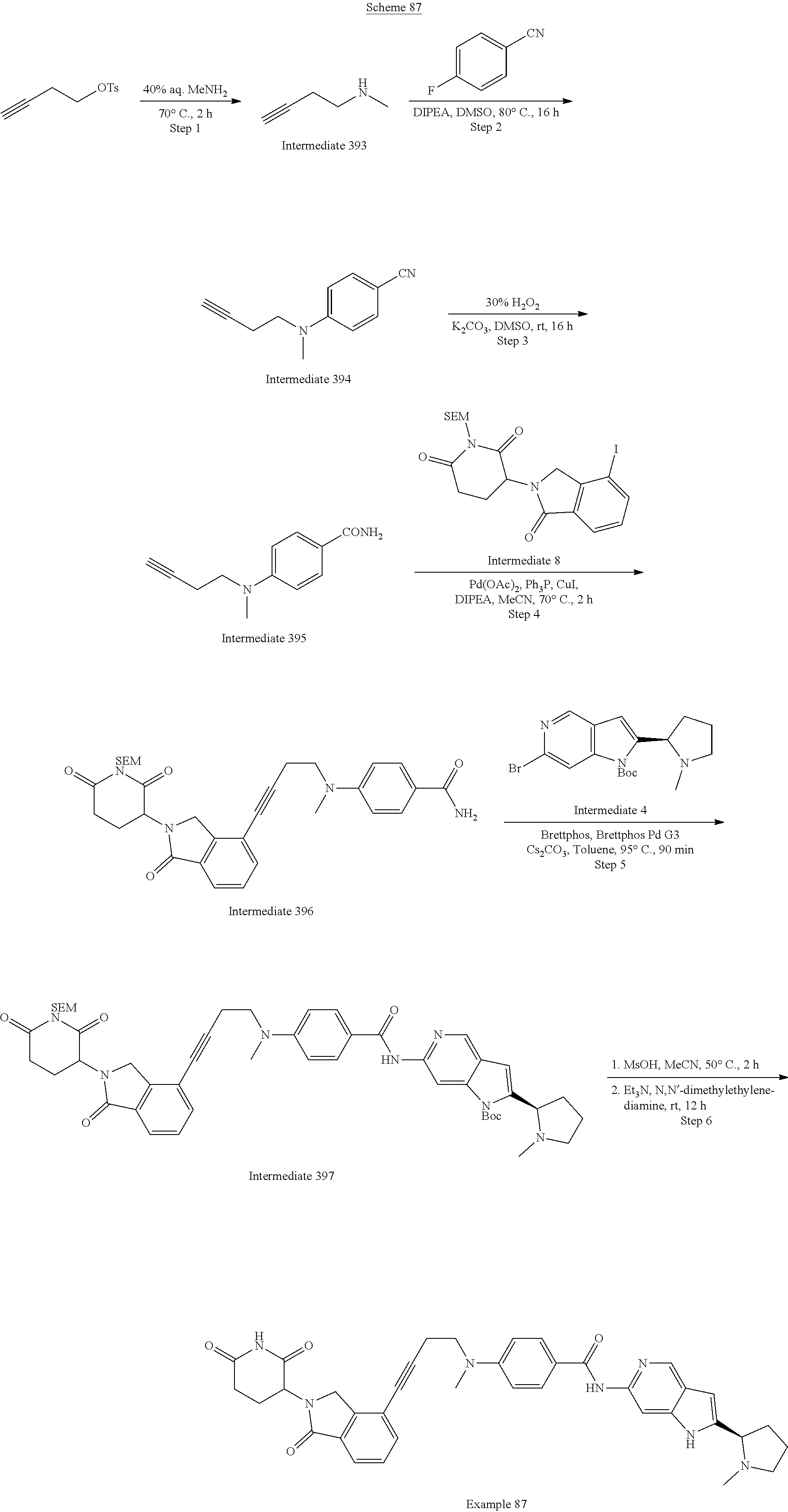

### Step 1

### Intermediate 393: N-methylbut-3-yn-1-amine

A solution of but-3-yn-1-yl 4-methylbenzenesulfonate (CAS No. 23418-85-1, 16.0 g, 71.3 mmol) in 40% methylamine in water (48 mL) was heated at 70° C. for 2 h. The reaction mixture was cooled then diluted with DCM (100 mL). The organic layer was separated, dried over anhydrous Na₂SO₄, filtered and concentrated in vacuo to afford N-methylbut-3-yn-1-amine (Intermediate 393, 3.6 g, 61%) as a white solid, which was used for the next step without further purification. ¹H NMR (400 MHz, CDCl₃) δ ppm 2.24-2.40 (m, 4H), 2.44 (s, 3H), 2.60-2.68 (m, 1H), 2.75 (t, J=6.42 Hz, 1H).

### Step 2

### Intermediate 394: 4-(but-3-yn-1-yl(methyl)amino)benzonitrile

To a solution of N-methylbut-3-yn-1-amine (Intermediate 393, 10.0 g, 100.0 mmol) and 4-fluorobenzonitrile (CAS No. 1194-02-1, 6.0 g, 50.0 mmol) in dimethyl sulfoxide (60 mL) was added N,N-diisopropylethylamine (40 mL, 200.0 mmol). The reaction mixture was heated at 80° C. for 16 h. After completion, the reaction mixture was poured into water (200 mL) and the resultant precipitate was collected by filtration and dried in vacuo. The crude material was purified by combi-flash chromatography, by eluting with 30% ethyl acetate in heptane, to afford 4-(but-3-yn-1-yl(methyl)amino)benzonitrile (Intermediate 394, 3.0 g, 30%) as an off-white solid. ¹H NMR (400 MHz, DMSO-d₆) δ ppm 2.22-2.30 (m, 1H), 2.39-2.44 (m, 2H), 3.01 (s, 3H), 3.59 (t, J=6.85 Hz, 2H), 6.78 (d, J=8.61 Hz, 2H), 7.53 (d, J=8.61 Hz, 2H). Mass spec m/z 185.5 [M+H]⁺.

### Step 3

### Intermediate 395: 4-(but-3-yn-1-yl(methyl)amino)benzamide

To a solution of 4-(but-3-yn-1-yl(methyl)amino)benzonitrile (Intermediate 394, 1.6 g, 8.7 mmol) in dimethyl sulfoxide (20 mL) was added potassium carbonate (1.8 g, 13 mmol) and hydrogen peroxide (0.80 mL, 26 mmol). The reaction mixture was stirred at room temperature for 16 h. The reaction mixture was then diluted with water (100 mL) and the resultant precipitate was collected by filtration and dried in vacuo to afford 4-(but-3-yn-1-yl(methyl)amino)benzamide (Intermediate 395, 0.63 g, 36%) as an off-white solid, which was used for the next step without further purification. ¹H NMR (400 MHz, DMSO-d₆) δ ppm 2.38-2.40 (m, 2H), 2.82-2.87 (m, 1H), 2.98 (s, 3H), 3.54-3.58 (m, 2H), 6.69 (d, J=8.61 Hz, 2H), 6.90 (br s, 1H), 7.61 (br s, 1H), 7.72 (d, J=8.22 Hz, 2H). Mass spec m/z 203.5 [M+H]⁺.

### Step 4

### Intermediate 396: 4-((4-(2-(2,6-dioxo-1-((2-(trimethylsilyl)ethoxy)methyl)piperidin-3-yl)-1-oxoisoindolin-4-yl) but-3-yn-1-yl)(methyl)amino)benzamide

To a suspension of 4-(but-3-yn-1-yl(methyl)amino)benzamide (Intermediate 395, 0.3 g, 1.0 mmol) and 3-(4-iodo-1-oxoisoindolin-2-yl)-1-((2-(trimethylsilyl)ethoxy)methyl)piperidine-2,6-dione (Intermediate 8, 0.5 g, 1.0 mmol) in acetonitrile (10 mL) was added Ph₃P (0.08 g, 0.30 mmol) and N,N-diisopropylethylamine (0.9 mL, 5.0 mmol). The reaction mixture was purged with argon for 15 min then palladium (II) acetate (0.02 g, 0.1 mmol) and copper (I) iodide (0.02 g, 0.1 mmol) were added. The reaction mixture was further purged with argon for 10 min and then heated at 70° C. for 2 h. After completion, the reaction mixture was filtered, and the filtrate was concentrated in vacuo. The crude material was purified by combi-flash chromatography, by eluting with 95% ethyl acetate in heptane, to afford 4-((4-(2-(2,6-dioxo-1-((2-(trimethylsilyl)ethoxy)methyl)piperidin-3-yl)-1-oxoisoindolin-4-yl)but-3-yn-1-yl)(methyl)amino)benzamide (Intermediate 396, 0.45 g, 80%) as light brown solid. ¹H NMR (400 MHz, DMSO-d₆) δ ppm −0.03 (s, 9H), 0.80-0.85 (m, 2H), 2.00-2.08 (m, 1H), 2.36-2.40 (m, 1H), 2.72-2.82 (m, 3H), 3.03 (s, 3H), 3.05-3.13 (m, 1H), 3.50-3.57 (m, 2H), 3.69-3.73 (m, 2H), 4.01-4.03 (m, 1H), 4.17-4.24 (m, 1H), 4.32-4.41 (m, 1H), 5.01-5.09 (m, 2H), 6.76 (d, J=9.07 Hz, 2H), 6.91 (br s, 1H), 7.49-7.53 (m, 1H), 7.59 (d, J=7.18 Hz, 1H), 7.62 (br s, 1H), 7.70-7.74 (m, 3H). Mass spec m/z 575.5 [M+H]⁺.

### Step 5

### Intermediate 397: tert-butyl-6-(4-((4-(2-(2,6-dioxo-1-((2-(trimethylsilyl)ethoxy)methyl)piperidin-3-yl)-1-oxoisoindolin-4-yl)but-3-yn-1-yl)(methyl)amino)benzamido)-2-((R)-1-methylpyrrolidin-2-yl)-1H-pyrrolo[3,2-c]pyridine-1-carboxylate

To a solution of 4-((4-(2-(2,6-dioxo-1-((2-(trimethylsilyl)ethoxy)methyl)piperidin-3-yl)-1-oxoisoindolin-4-yl)but-3-yn-1-yl)(methyl)amino)benzamide (Intermediate 396, 0.50 g, 0.87 mmol) in 1,4-dioxane (10 mL) was added tert-butyl-(R)-6-bromo-2-(1-methylpyrrolidin-2-yl)-1H-pyrrolo[3,2-c]pyridine-1-carboxylate (Intermediate 4, 0.49 g, 1.30 mmol) followed by cesium carbonate (0.85 g, 2.61 mmol). The reaction mixture was purged with argon for 15 min, then BrettPhos Pd G3 (0.04 g, 0.43 mmol) followed by BrettPhos (0.24 g, 0.43 mmol) were added. The reaction mixture was further purged with argon for 10 min then heated at 95° C. for 90 min. After completion, the reaction mixture was filtered, and the filtrate was concentrated in vacuo. The crude material was purified by combi-flash chromatography, by eluting with 10% MeOH in DCM, to afford tert-butyl-6-(4-((4-(2-(2,6-dioxo-1-((2-(trimethylsilyl)ethoxy)methyl)piperidin-3-yl)-1-oxoisoindolin-4-yl)but-3-yn-1-yl)(methyl)amino)benzamido)-2-((R)-1-methylpyrrolidin-2-yl)-1H-pyrrolo[3,2-c]pyridine-1-carboxylate (Intermediate 397, 0.40 g, 53%) as an off-white solid. ¹H NMR (400 MHz, DMSO-d₆) δ ppm −0.05 (s, 9H), 0.78-0.83 (m, 2H), 1.60-1.66 (m, 1H), 1.70 (s, 9H), 1.72-1.79 (m, 2H), 2.30-2.33 (m, 1H), 2.35 (s, 3H), 2.36-2.41 (m, 2H), 2.74-2.83 (m, 3H), 3.08 (s, 3H), 3.09-3.16 (m, 2H), 3.47-3.52 (m, 2H), 3.74-3.77 (m, 2H), 3.87-3.94 (m, 1H), 4.16-4.21 (m, 1H), 4.33-4.37 (m, 1H), 4.98-5.07 (m, 2H), 5.18-5.20 (m, 1H), 5.76 (s, 1H), 6.73 (s, 1H), 6.83 (d, J=8.61 Hz, 2H), 7.48-7.56 (m, 1H), 7.61 (d, J=7.43 Hz, 1H), 7.71 (d, J=7.43 Hz, 1H), 7.97 (d, J=8.61 Hz, 2H), 8.56 (s, 1H), 8.91 (s, 1H), 10.22 (br s, 1H). Mass spec m/z 874.1 [M+H]⁺.

### Step 6

### Example 87: 4-((4-(2-(2,6-dioxopiperidin-3-yl)-1-oxoisoindolin-4-yl)but-3-yn-1-yl) (methyl)amino)-N-(2-((R)-1-methylpyrrolidin-2-yl)-1H-pyrrolo[3,2-c]pyridin-6-yl)benzamide

To a solution of tert-butyl-6-(4-((4-(2-(2,6-dioxo-1-((2-(trimethylsilyl)ethoxy)methyl)piperidin-3-yl)-1-oxoisoindolin-4-yl)but-3-yn-1-yl)(methyl)amino)benzamido)-2-((R)-1-methylpyrrolidin-2-yl)-1H-pyrrolo[3,2-c]pyridine-1-carboxylate (Intermediate 397, 0.45 g, 0.51 mmol) in acetonitrile (7 mL) was added methanesulfonic acid (0.34 mL, 5.14 mmol) and the reaction mixture was heated at 50° C. for 2 h. The reaction mixture was cooled to room temperature, then N,N′-dimethylethylenediamine (0.29 mL, 2.57 mmol) followed by triethylamine (1.44 mL, 10.3 mmol) were added and the mixture was stirred at room temperature for 12 h. The reaction mixture was diluted with ice cold water (10 mL) the resultant precipitate was collected by filtration and dried in vacuo to afford the crude compound. The crude material was purified by preparative HPLC (Method A) to afford 4-((4-(2-(2,6-dioxopiperidin-3-yl)-1-oxoisoindolin-4-yl)but-3-yn-1-yl)(methyl)amino)-N-(2-((R)-1-methylpyrrolidin-2-yl)-1H-pyrrolo[3,2-c]pyridin-6-yl)benzamide (Example 87, 0.17 g, 53%) as an off-white solid. ¹H NMR (400 MHz, DMSO-d₆) δ ppm 1.77-1.95 (m, 3H), 1.96-2.04 (m, 1H), 2.10-2.14 (m, 1H), 2.16 (s, 3H), 2.26-2.33 (m, 1H), 2.37-2.46 (m, 1H), 2.56-2.64 (m, 1H), 2.76-2.798 (m, 2H), 2.85-2.97 (m, 1H), 3.08 (s, 3H), 3.10-3.18 (m, 1H), 3.26-3.31 (m, 1H), 3.73-3.81 (m, 2H), 4.23-4.31 (m, 1H), 4.35-4.43 (m, 1H), 5.09-5.14 (m, 1H), 6.37 (s, 1H), 6.83 (d, J=9.01 Hz, 2H), 7.48-7.54 (m, 1H), 7.57-7.62 (m, 1H), 7.70 (dd, J=7.50, 0.75 Hz, 1H), 7.96 (d, J=9.01 Hz, 2H), 8.17 (s, 1H), 8.47 (s, 1H), 9.99 (s, 1H), 11.07 (s, 1H), 11.31 (s, 1H). Mass spec m/z 644.0 [M+H]⁺.

### Example 88 was Prepared Following Scheme 88

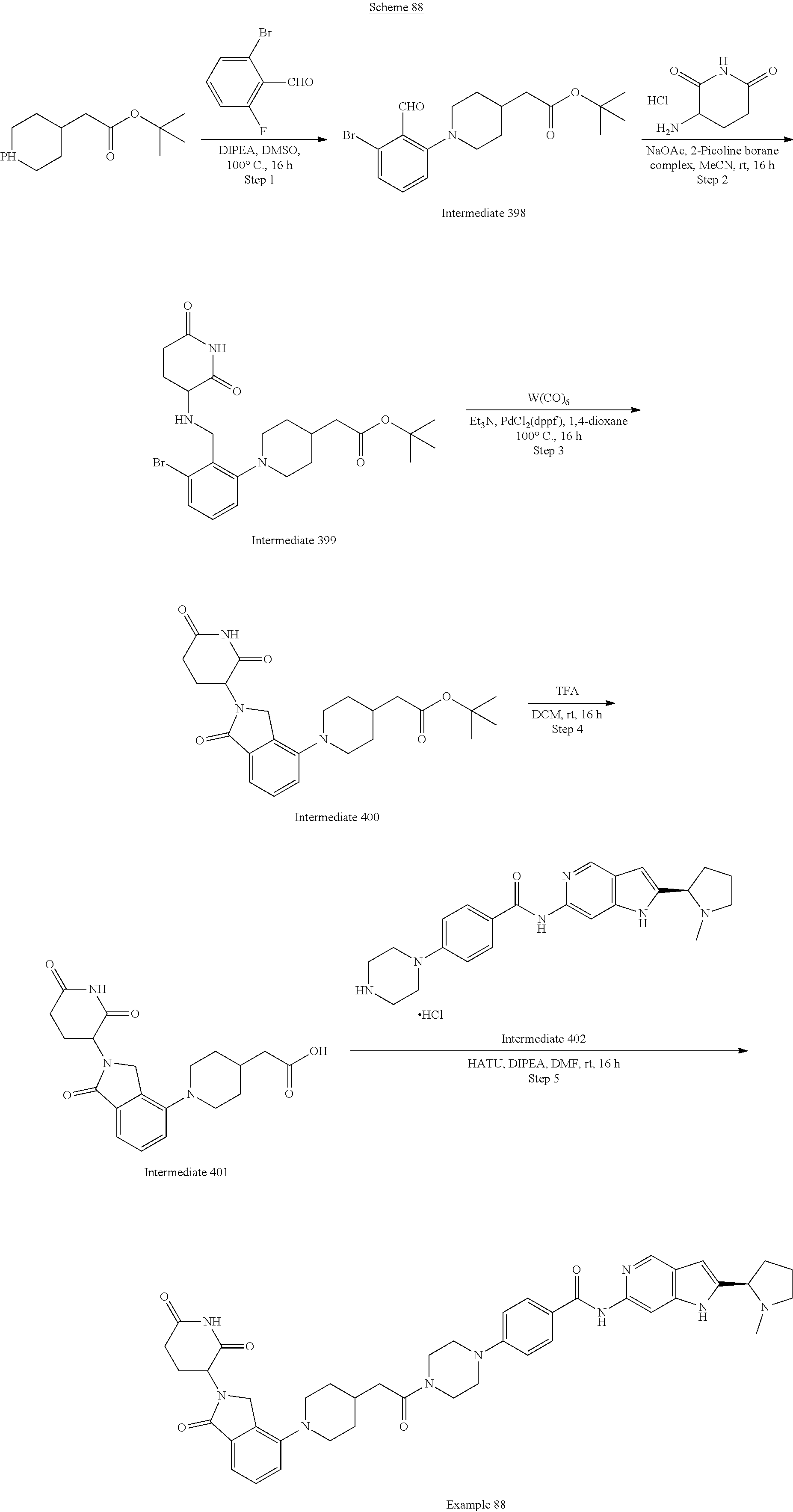

### Step 1

### Intermediate 398: tert-butyl-2-(1-(3-bromo-2-formylphenyl)piperidin-4-yl) acetate

To a solution of tert-butyl-2-(piperidin-4-yl) acetate (CAS No. 180182-07-4, 5.0 g, 24.3 mmol) in dimethyl sulfoxide (35 mL) was added 2-bromo-6-fluorobenzaldehyde (CAS No. 360575-28-6, 5.14 g, 24.3 mmol) followed by N,N-diisopropylethylamine (17 mL, 97.3 mmol). The reaction mixture was heated at 100° C. for 16 h. After completion, the reaction mixture was diluted with water (200 mL) and extracted with ethyl acetate (2×200 mL). The combined organic layers were dried over anhydrous Na₂SO₄, and concentrated in vacuo. The crude material was purified by combi-flash chromatography, by eluting with 15% ethyl acetate in heptane, to afford tert-butyl-2-(1-(3-bromo-2-formylphenyl)piperidin-4-yl) acetate (Intermediate 398, 5.0 g, 46%) as a yellow solid. ¹H NMR (400 MHz, DMSO-d₆) δ ppm 1.36-1.40 (m, 2H), 1.41 (s, 9H), 1.71-1.76 (m, 2H), 1.77-1.82 (m, 1H), 2.17-2.21 (m, 2H), 2.82-2.88 (m, 2H), 3.12-3.18 (m, 2H), 7.22 (d, J=8.31 Hz, 1H), 7.31 (d, J=7.93 Hz, 1H), 7.41 (t, J=7.93 Hz, 1H), 10.02 (s, 1H).

### Step 2

### Intermediate 399: tert-butyl-2-(1-(3-bromo-2-(((2,6-dioxopiperidin-3-yl)amino)methyl)phenyl)piperidin-4-yl) acetate

To a solution of tert-butyl 2-(1-(3-bromo-2-formylphenyl)piperidin-4-yl) acetate (Intermediate 398, 4.0 g, 10.5 mmol) and 3-aminopiperidine-2,6-dione hydrochloride (CAS No. 24666-56-6, 2.58 g, 15.7 mmol) in acetonitrile (70 mL) was added sodium acetate (1.30 g, 15.7 mmol) followed by borane-2-picoline complex (2.94 g, 26.2 mmol). The reaction mixture was stirred at room temperature for 16 h. After completion, the reaction mixture was filtered and concentrated in vacuo. The crude material was purified by combi-flash chromatography, by eluting with 50% ethyl acetate in heptane, to afford tert-butyl-2-(1-(3-bromo-2-(((2,6-dioxopiperidin-3-yl)amino)methyl)phenyl)piperidin-4-yl) acetate (Intermediate 399, 3.2 g, 62%) as an off-white solid. ¹H NMR (400 MHz, DMSO-d₆) δ ppm 1.41 (s, 9H), 1.68-1.77 (m, 3H), 2.18-2.20 (m, 2H), 2.28-2.31 (m, 1H), 2.52-2.58 (m, 2H), 2.73-2.79 (m, 1H), 3.01-3.07 (m, 1H), 3.20-3.21 (m, 2H), 3.85-3.99 (m, 2H), 5.75 (s, 1H), 7.15-7.24 (m, 2H), 7.31-7.46 (m, 1H), 10.77 (br s, 1H).

### Step 3

### Intermediate 400: tert-butyl-2-(1-(2-(2,6-dioxopiperidin-3-yl)-1-oxoisoindolin-4-yl)piperidin-4-yl) acetate

To a solution of tert-butyl-2-(1-(3-bromo-2-(((2,6-dioxopiperidin-3-yl)amino)methyl)phenyl)piperidin-4-yl) acetate (Intermediate 399, 3.2 g, 6.5 mmol) in 1,4-dioxane (30 mL) was added triethylamine (2.7 mL, 19 mmol) at room temperature. The reaction mixture was purged with argon for 15 min then Pd(dppf)Cl₂ (1.0 g., 1.3 mmol), Xantphos (0.77 g, 1.3 mmol) and tungsten hexacarbonyl (1.2, 3.2 mmol) were added. The reaction mixture was further purged with argon for 10 min and heated at 100° C. for 16 h. The reaction mixture was concentrated in vacuo. The crude material was purified by combi-flash chromatography, by eluting with 12% MeOH in DCM, to afford tert-butyl-2-(1-(2-(2,6-dioxopiperidin-3-yl)-1-oxoisoindolin-4-yl)piperidin-4-yl) acetate (Intermediate 400, 2.6 g, 91%) as an off-white solid. ¹H NMR (400 MHz, DMSO-d₆) δ ppm 1.31-1.37 (m, 3H), 1.41 (s, 9H), 1.74-1.77 (m, 4H), 1.93-2.04 (m, 1H), 2.16-2.19 (m, 2H), 2.67-2.79 (m, 3H), 2.87-2.96 (m, 1H), 3.35-3.40 (m, 2H), 4.25-4.32 (m, 1H), 4.40-4.46 (m, 1H), 7.15 (d, J=7.93 Hz, 1H), 7.25-7.33 (m, 1H), 7.40-7.46 (m, 1H), 10.96 (s, 1H). Mass spec m/z 442.4 [M+H]⁺.

### Step 4

### Intermediate 401: 2-(1-(2-(2,6-dioxopiperidin-3-yl)-1-oxoisoindolin-4-yl)piperidin-4-yl) acetic acid

To a solution of tert-butyl-2-(1-(2-(2,6-dioxopiperidin-3-yl)-1-oxoisoindolin-4-yl)piperidin-4-yl) acetate (Intermediate 400, 1.5 g, 3.4 mmol) in dichloromethane (15 mL) was added trifluoroacetic acid (10 mL, 130.0 mmol). The reaction mixture was stirred at room temperature for 16 h, then concentrated in vacuo. The crude material was triturated with diethyl ether (2×10 mL) and dried in vacuo to afford 2-(1-(2-(2,6-dioxopiperidin-3-yl)-1-oxoisoindolin-4-yl)piperidin-4-yl)acetic acid (Intermediate 401, 1.5 g, 70%) as a red solid. ¹H NMR (400 MHz, DMSO-d₆) δ ppm 1.17-1.20 (m, 2H), 1.28-1.58 (m, 2H), 1.75-1.86 (m, 2H), 1.94-2.06 (m, 1H), 2.18-2.25 (m, 1H), 2.57-2.67 (m, 1H), 2.72-2.96 (m, 2H), 3.03-3.18 (m, 4H), 4.25-4.35 (m, 1H), 4.40-4.50 (m, 1H), 5.09-5.14 (m, 1H), 7.22 (d, J=7.83 Hz, 1H), 7.33 (d, J=7.04 Hz, 1H), 7.40-7.49 (m, 1H), 9.01 (br s, 1H), 10.98 (br s, 1H). Mass spec m/z 386.1 [M+H]⁺.

### Step 5

### Example 88: 4-(4-(2-(1-(2-(2,6-dioxopiperidin-3-yl)-1-oxoisoindolin-4-yl)piperidin-4-yl)acetyl) piperazin-1-yl)-N-(2-((R)-1-methylpyrrolidin-2-yl)-1H-pyrrolo[3,2-c]pyridin-6-yl)benzamide

To a solution of 2-(1-(2-(2,6-dioxopiperidin-3-yl)-1-oxoisoindolin-4-yl)piperidin-4-yl) acetic acid (Intermediate 401, 0.25 g, 0.25 mmol) in dimethylformamide (3 mL) was added HATU (0.28 g, 0.75 mmol) and N,N-diisopropylethylamine (0.5 mL, 2.50 mmol) and the reaction mixture was stirred at room temperature for 10 min. tert-Butyl-(R)-2-(1-methylpyrrolidin-2-yl)-6-(4-(piperazin-1-yl)benzamido)-1H-pyrrolo[3,2-c]pyridine-1-carboxylate hydrochloride (Intermediate 402, 0.3 g, 0.55 mmol) was then added and the reaction mixture was stirred at room temperature for 16 h. The reaction mixture was quenched with ice cold water (50 mL), the resultant precipitate was collected by filtration and dried in vacuo. The crude material was purified by preparative HPLC (Method A) to afford 4-(4-(2-(1-(2-(2,6-dioxopiperidin-3-yl)-1-oxoisoindolin-4-yl)piperidin-4-yl)acetyl)piperazin-1-yl)-N-(2-((R)-1-methylpyrrolidin-2-yl)-1H-pyrrolo[3,2-c]pyridin-6-yl)benzamide (Example 88, 0.08 g, 21%) as an off-white solid. ¹H NMR (400 MHz, DMSO-d₆) δ ppm 1.34-1.43 (m, 2H), 1.87-1.94 (m, 6H), 1.95-2.04 (m, 1H), 2.10-2.14 (m, 1H), 2.16 (s, 3H), 2.22-2.29 (m, 1H), 2.33-2.38 (m, 2H), 2.54-2.60 (m, 2H), 2.71-2.79 (m, 2H), 2.87-2.96 (m, 1H), 3.11-3.15 (m, 1H), 3.26-3.29 (m, 4H), 3.36-3.42 (m, 3H), 3.61-3.68 (m, 4H), 4.25-4.33 (m, 1H), 4.39-4.47 (m, 1H), 5.08-5.13 (m, 1H), 6.37 (s, 1H), 7.01 (d, J=9.05 Hz, 2H), 7.17 (d, J=7.82 Hz, 1H), 7.29 (d, J=7.46 Hz, 1H), 7.40-7.44 (m, 1H), 7.99 (d, J=8.93 Hz, 2H), 8.17 (s, 1H), 8.47 (s, 1H), 10.13 (s, 1H), 10.98 (s, 1H), 11.32 (s, 1H). Mass spec m/z 772.1 [M+H]⁺.

### Intermediate 402 was Synthesised Following Scheme 89

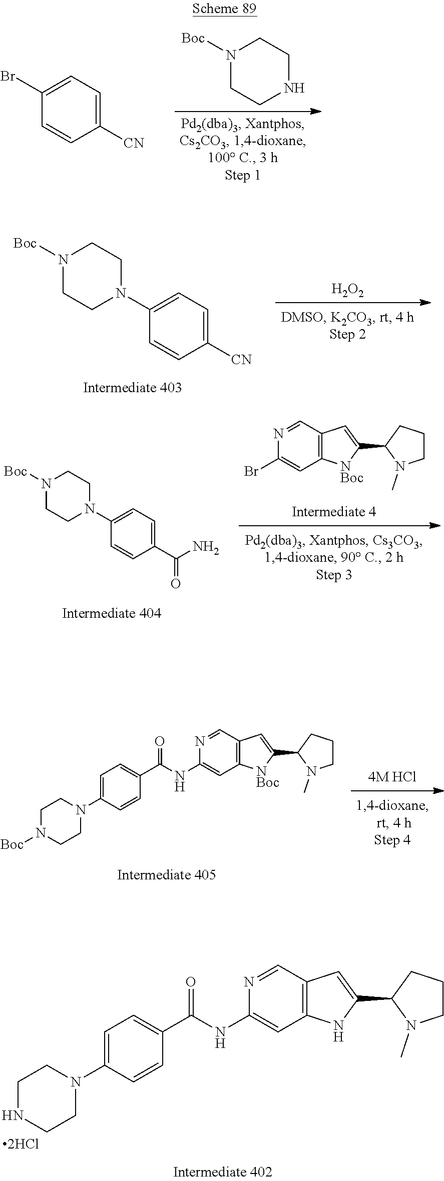

### Step 1

### Intermediate 403: tert-butyl 4-(4-cyanophenyl)piperazine-1-carboxylate

To a solution of 4-bromobenzonitrile (CAS No. 623-00-7, 2.5 g, 14.0 mmol) in 1,4-dioxane (25 mL) was added tert-butyl piperazine-1-carboxylate (CAS No. 57260-71-6, 2.6 g, 14.0 mmol) followed by cesium carbonate (13.0 g, 41.0 mmol). The reaction mixture was purged with argon gas for 15 min then Pd₂(dba)₃ (1.3 g, 1.4 mmol) and Xantphos (1.6 g, 2.7 mmol) were added. The reaction mixture was purged with argon gas for another 10 min and stirred at 100° C. for 3 h. After completion, the reaction mixture was filtered through a celite bed, the filter pad was washed with ethyl acetate (50 mL) and the filtrate was concentrated in vacuo. The crude material was purified by combi-flash chromatography, by eluting with 90% ethyl acetate in heptane, to afford tert-butyl 4-(4-cyanophenyl)piperazine-1-carboxylate (Intermediate 403, 1.5 g, 38%) as a brown solid. ¹H NMR (400 MHz, DMSO-d₆) δ ppm 1.42 (s, 9H), 3.18-3.21 (m, 4H), 3.42-3.46 (m, 4H), 7.18 (d, J=7.46 Hz, 1H), 7.26-7.29 (m, 1H), 7.33-7.35 (m, 1H), 7.37-7.42 (m, 1H). Mass spec m/z 288.1 [M+H]⁺.

### Step 2

### Intermediate 404: tert-butyl 4-(4-carbamoylphenyl)piperazine-1-carboxylate

To a cooled (0° C.) solution of tert-butyl 4-(4-cyanophenyl)piperazine-1-carboxylate (Intermediate 403, 3.0 g, 10.4 mmol) in dimethyl sulfoxide (30 mL) was added potassium carbonate (4.3 g, 31.32 mmol) followed by 30% hydrogen peroxide (0.96 mL, 31.32 mmol) and the reaction mixture was stirred at room temperature for 4 h. After completion, the reaction mixture was quenched with ice cold water (200 mL), the resultant precipitate was collected by filtration and dried in vacuo to afford tert-butyl 4-(4-carbamoylphenyl)piperazine-1-carboxylate (Intermediate 404, 2.5 g, 78%) as a white solid, which was used for the next step without further purification. ¹H NMR (400 MHz, DMSO-d₆) δ ppm 1.42 (s, 9H), 3.10-3.17 (m, 4H), 3.43-3.50 (m, 4H), 7.09 (d, J=7.18 Hz, 1H), 7.25-7.35 (m, 3H), 7.42 (br s, 1H), 7.90 (br s, 1H). Mass spec m/z 306.1 [M+H]⁺.

### Step 3

### Intermediate 405: tert-butyl (R)-6-(4-(4-(tert-butoxycarbonyl)piperazin-1-yl)benzamido)-2-(1-methylpyrrolidin-2-yl)-1H-pyrrolo[3,2-c]pyridine-1-carboxylate

To a solution of tert-butyl 4-(4-carbamoylphenyl)piperazine-1-carboxylate (Intermediate 404, 0.64 g, 2.10 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 1.0 g, 2.63 mmol) followed by cesium carbonate (2.57 g, 7.88 mmol). The reaction mixture was purged with argon for 15 min then Pd₂(dba)₃ (0.24 g, 0.26 mmol) and Xantphos (0.47 g, 0.78 mmol) were added. The reaction mixture was further purged with argon for 10 min and heated at 100° C. for 2 h. After completion, reaction mixture was filtered through a celite bed and the filter pad was washed with ethyl acetate. The filtrate was concentrated in vacuo and the crude material purified by combi-flash chromatography, by eluting with 100% ethyl acetate, to afford tert-butyl (R)-6-(4-(4-(tert-butoxycarbonyl)piperazin-1-yl)benzamide)-2-(1-methylpyrrolidin-2-yl)-1H-pyrrolo[3,2-c]pyridine-1-carboxylate (Intermediate 405, 1.5 g, 94%) as a yellow solid. ¹H NMR (401 MHz, DMSO-d₆) δ ppm 1.42 (s, 9H) 1.65-1.68 (m, 1H) 1.69 (s, 9H) 2.32-2.35 (m, 1H) 2.35 (s, 3H) 2.36-2.39 (m, 1H) 3.12-3.16 (m, 5H) 3.45-3.52 (m, 5H) 3.89-3.94 (m, 1H) 4.00-4.06 (m, 1H) 6.75 (s, 1H) 7.07-7.12 (m, 1H) 7.13-7.17 (m, 1H) 7.41-7.48 (m, 2H) 7.91 (s, 1H) 8.59 (s, 1H) 10.72 (br s, 1H). Mass spec m/z 605.3 [M+H]⁺.

### Step 4

### Intermediate 402: (R)—N-(2-(1-methylpyrrolidin-2-yl)-1H-pyrrolo[3,2-c]pyridin-6-yl)-4-(piperazin-1-yl)benzamide dihydrochloride

To a cooled (0° C.) solution of tert-butyl (R)-6-(4-(4-(tert-butoxycarbonyl)piperazin-1-yl)benzamido)-2-(1-methylpyrrolidin-2-yl)-1H-pyrrolo[3,2-c]pyridine-1-carboxylate (Intermediate 405, 1.5 g, 2.5 mmol) in 1,4-dioxane (25 mL) was added 4M hydrochloric acid in dioxane (25 mL) and the reaction mixture was stirred at room temperature for 4 h. After completion, the reaction mixture was concentrated in vacuo to afford (R)—N-(2-(1-methylpyrrolidin-2-yl)-1H-pyrrolo[3,2-c]pyridin-6-yl)-4-(piperazin-1-yl)benzamide dihydrochloride (Intermediate 402, 1.0 g, 100%) as a yellow solid, which was used for the next step without further purification. Mass spec m/z 405.1 [M+H]⁺.

### Example 89 was Prepared Following Scheme 90

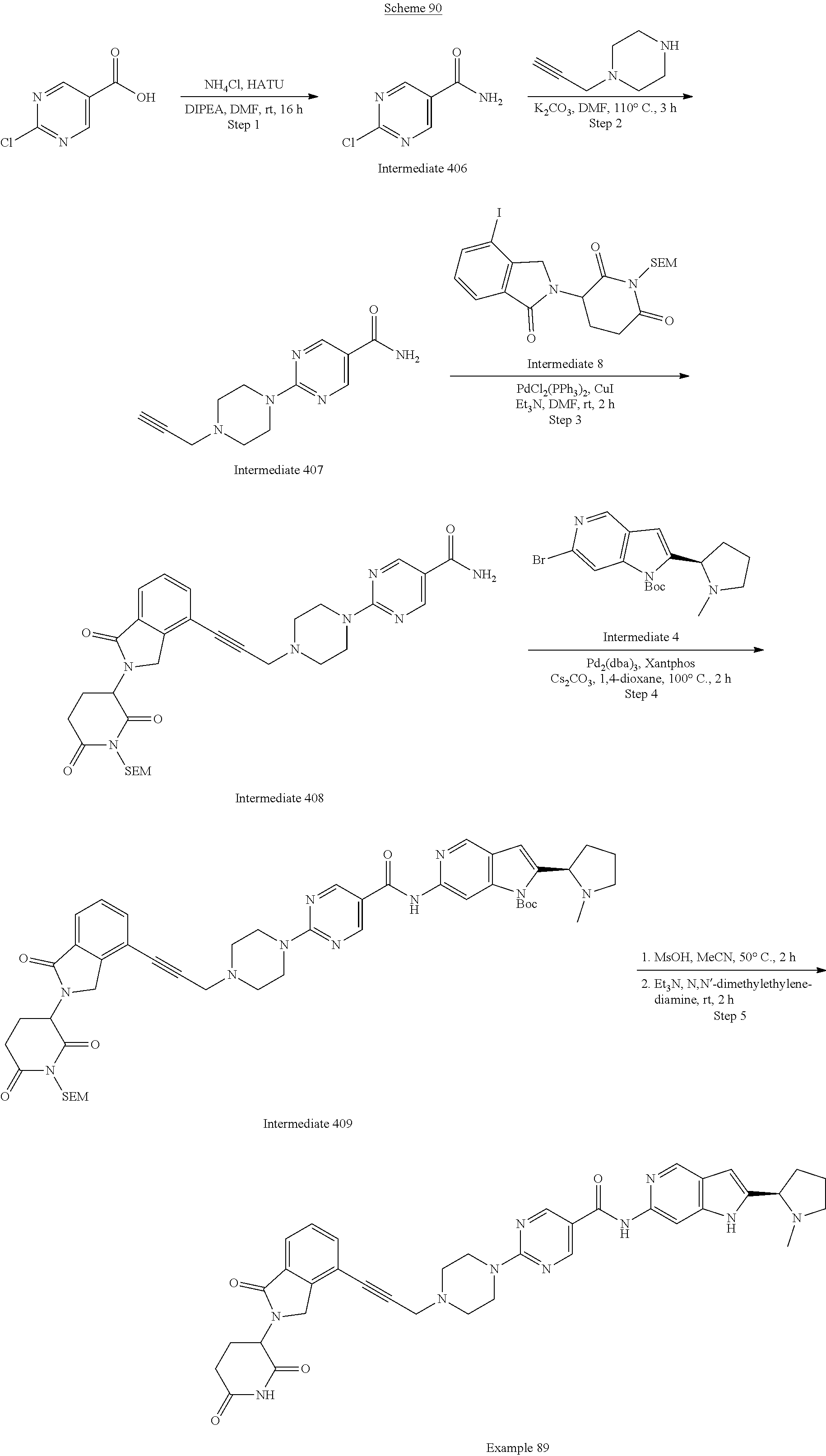

### Step 1

### Intermediate 406: 2-chloropyrimidine-5-carboxamide

To a solution of 2-chloropyrimidine-5-carboxylic acid (CAS No. 374068-01-6, 11.0 g, 69.4 mmol) in dimethylformamide (100 mL) was added HATU (41.7 g, 104 mmol) and N,N-diisopropylethylamine (48.5 mL, 278 mmol). Ammonium chloride (18.6 g, 347 mmol) was then added, and the reaction mixture was stirred at room temperature for 16 h then quenched with ice cold water (200 mL). The resultant precipitate was collected by filtration and dried in vacuo to afford 2-chloropyrimidine-5-carboxamide (Intermediate 406, 10 g, 91%) as a white solid, which was used for the next step without further purification. ¹H NMR (400 MHz, DMSO-d₆) δ ppm 7.86 (s, 1H), 8.25 (s, 1H), 9.10 (br s, 2H). Mass spec m/z 158.0 [M+H]⁺.

### Step 2

### Intermediate 407: 2-(4-(prop-2-yn-1-yl)piperazin-1-yl)pyrimidine-5-carboxamide

To a cooled (0° C.) solution of 2-chloropyrimidine-5-carboxamide (Intermediate 406, 2.0 g, 12.7 mmol) and 1-(prop-2-yn-1-yl)piperazine (CAS No. 52070-67-4, 1.58 g, 12.74 mmol) in dimethylformamide (25 mL) was added potassium carbonate (5.28 g, 38.2 mmol). The reaction mixture was stirred at 110° C. for 3 h then concentrated in vacuo. The crude material was purified by combi-flash chromatography, by eluting with 40% ethyl acetate in heptane, to afford 2-(4-(prop-2-yn-1-yl)piperazin-1-yl)pyrimidine-5-carboxamide (Intermediate 407, 0.56 g, 18%) as an off-white solid. Mass spec m/z 246.0 [M+H]⁺.

### Step 3

### Intermediate 408: 2-(4-(3-(2-(2,6-dioxo-1-((2-(trimethylsilyl)ethoxy)methyl)piperidin-3-yl)-1-oxoisoindolin-4-yl)prop-2-yn-1-yl)piperazin-1-yl)pyrimidine-5-carboxamide

To a solution of 2-(4-(prop-2-yn-1-yl)piperazin-1-yl)pyrimidine-5-carboxamide (Intermediate 407, 0.5 g, 2.03 mmol) in dimethylformamide (2 mL) was added 3-(4-iodo-1-oxoisoindolin-2-yl)-1-((2-(trimethylsilyl)ethoxy)methyl)piperidine-2,6-dione (Intermediate 8, 1.02 g, 2.03 mmol) followed by triethylamine (11.1 mL, 79.5 mmol). The reaction mixture was purged with argon for 15 min then copper (I) iodide (0.04 g, 0.20 mmol) and PdCl₂(PPh₃)₂ (0.14 g, 0.20 mmol) were added. The reaction mixture was further purged with argon for 10 min and then stirred at room temperature for 2 h. The reaction mixture was diluted with water (90 mL) and extracted with ethyl acetate (2×80 mL). The combined organic layers were dried over anhydrous Na₂SO₄ and concentrated in vacuo. The crude material was purified by combi-flash chromatography, by eluting with 5% MeOH in DCM, to afford 2-(4-(3-(2-(2,6-dioxo-1-((2-(trimethylsilyl)ethoxy)methyl)piperidin-3-yl)-1-oxoisoindolin-4-yl)prop-2-yn-1-yl)piperazin-1-yl)pyrimidine-5-carboxamide (Intermediate 408, 0.4 g, 32%) as a brown solid. ¹H NMR (400 MHz, DMSO-d₆) δ ppm −0.03 (s, 9H), 0.81-0.85 (m, 2H), 2.01-2.08 (m, 1H), 2.57-2.63 (m, 4H), 2.74-2.81 (m, 2H), 3.01-3.09 (m, 1H), 3.46-3.58 (m, 2H), 3.65 (s, 2H), 3.82-3.90 (m, 4H), 4.24-4.33 (m, 1H), 4.25-4.32 (m, 1H), 5.01-5.09 (m, 2H), 5.21-5.26 (m, 1H), 7.27 (s, 1H), 7.54 (t, J=7.46 Hz, 1H), 7.70 (d, J=8.0 Hz, 1H), 7.75 (d, J=7.46 Hz, 1H), 7.83 (s, 1H), 8.77 (br s, 2H). Mass spec m/z 618.3 [M+H]⁺.

### Step 4

### Intermediate 409: tert-butyl 6-(2-(4-(3-(2-(2,6-dioxo-1-((2-(trimethylsilyl)ethoxy)methyl)piperidin-3-yl)-1-oxoisoindolin-4-yl)prop-2-yn-1-yl)piperazin-1-yl)pyrimidine-5-carboxamido)-2-((R)-1-methylpyrrolidin-2-yl)-1H-pyrrolo[3,2-c]pyridine-1-carboxylate

To a solution of 2-(4-(3-(2-(2,6-dioxo-1-((2-(trimethylsilyl) ethoxy)methyl)piperidin-3-yl)-1-oxoisoindolin-4-yl)prop-2-yn-1-yl)piperazin-1-yl)pyrimidine-5-carboxamide (Intermediate 408, 0.4 g, 0.65 mmol) in 1,4-dioxane (4 mL) was added tert-butyl (R)-6-bromo-2-(1-methylpyrrolidin-2-yl)-1H-pyrrolo[3,2-c]pyridine-1-carboxylate (Intermediate 4, 0.31 g, 0.81 mmol) followed by cesium carbonate (0.64 g, 1.95 mmol). The reaction mixture was purged with argon for 15 min, then Pd₂(dba)₃ (0.09 g, 0.10 mmol) followed by Xantphos (0.45 g, 0.8 mmol) were added. The reaction mixture was further purged with argon for 10 min and stirred at 100° C. for 2 h. The reaction mixture was concentrated in vacuo and the crude material was purified by combi-flash chromatography, by eluting with 5% MeOH in DCM, to afford tert-butyl 6-(2-(4-(3-(2-(2,6-dioxo-1-((2-(trimethylsilyl)ethoxy)methyl)piperidin-3-yl)-1-oxoisoindolin-4-yl)prop-2-yn-1-yl)piperazin-1-yl)pyrimidine-5-carboxamido)-2-((R)-1-methylpyrrolidin-2-yl)-1H-pyrrolo[3,2-c]pyridine-1-carboxylate (Intermediate 409, 0.45 g, 61%) as a yellow solid. ¹H NMR (400 MHz, DMSO-d₆) δ ppm −0.05 (s, 9H), 0.74-0.84 (m, 2H), 1.06-1.11 (m, 2H), 1.56-1.62 (m, 2H), 1.69 (s, 9H), 1.86-1.96 (m, 2H), 1.98-2.07 (m, 3H), 2.29-2.33 (m, 2H), 2.35 (s, 3H), 2.38-2.42 (m, 1H), 2.59-2.66 (m, 4H), 3.10-3.16 (m, 2H), 3.17 (s, 2H), 3.67-3.71 (m, 2H), 3.86-3.96 (m, 4H), 4.49-4.53 (m, 1H), 5.02-5.06 (m, 1H), 6.75 (s, 1H), 7.45-7.57 (m, 2H), 7.64-7.76 (m, 2H), 8.58 (s, 1H), 8.91 (s, 1H), 8.96 (m, 1H), 10.73 (br s, 1H).

### Step 5

### Example 89: 2-(4-(3-(2-(2,6-dioxopiperidin-3-yl)-1-oxoisoindolin-4-yl)prop-2-yn-1-yl)piperazin-1-yl)-N-(2-((R)-1-methylpyrrolidin-2-yl)-1H-pyrrolo[3,2-c]pyridin-6-yl)pyrimidine-5-carboxamide

To a solution of tert-butyl 6-(2-(4-(3-(2-(2,6-dioxo-1-((2-(trimethylsilyl)ethoxy)methyl)piperidin-3-yl)-1-oxoisoindolin-4-yl)prop-2-yn-1-yl)piperazin-1-yl)pyrimidine-5-carboxamido)-2-((R)-1-methylpyrrolidin-2-yl)-1H-pyrrolo[3,2-c]pyridine-1-carboxylate (Intermediate 409, 0.40 g, 0.43 mmol) in acetonitrile (4 mL) was added methanesulfonic acid (0.28 mL, 4.36 mmol) and the reaction mixture was stirred at 50° C. for 2 h. The reaction mixture was cooled to room temperature, then N,N′-dimethylethylenediamine (0.52 mL, 4.36 mmol) followed by triethylamine (1.22 mL, 8.72 mmol) were added and the mixture was stirred at room temperature for 2 h. The reaction mixture was concentrated in vacuo and the resultant residue was triturated with diethyl ether (2×50 mL) followed by n-pentane (2×40 mL) and dried in vacuo to obtain the crude compound. The crude material was purified by preparative HPLC (Method A) to afford 2-(4-(3-(2-(2,6-dioxopiperidin-3-yl)-1-oxoisoindolin-4-yl)prop-2-yn-1-yl)piperazin-1-yl)-N-(2-((R)-1-methylpyrrolidin-2-yl)-1H-pyrrolo[3,2-c]pyridin-6-yl)pyrimidine-5-carboxamide (Example 89, 0.02 g, 9%) as an off-white solid. ¹H NMR (400 MHz, DMSO-d₆) δ ppm 1.81-1.95 (m, 3H), 1.97-2.02 (m, 1H), 2.13-2.15 (m, 1H), 2.16 (s, 3H), 2.24-2.30 (m, 1H), 2.39-2.46 (m, 1H), 2.52-2.58 (m, 2H), 2.60-2.67 (m, 4H), 2.85-2.96 (m, 1H), 3.10-3.18 (m, 1H), 3.67 (s, 2H), 3.87-3.94 (m, 4H), 4.30-4.37 (m, 1H), 4.43-4.50 (m, 1H), 5.09-5.14 (m, 1H), 6.38 (s, 1H), 7.50-7.57 (m, 1H), 7.69-7.74 (m, 2H), 8.16 (s, 1H), 8.48 (s, 1H), 8.96 (s, 2H), 10.47 (br s, 1H), 10.98 (br s, 1H), 11.35 (br s, 1H). Mass spec m/z 687.0 [M+H]⁺.

### Example 91 was Prepared Following Scheme 92

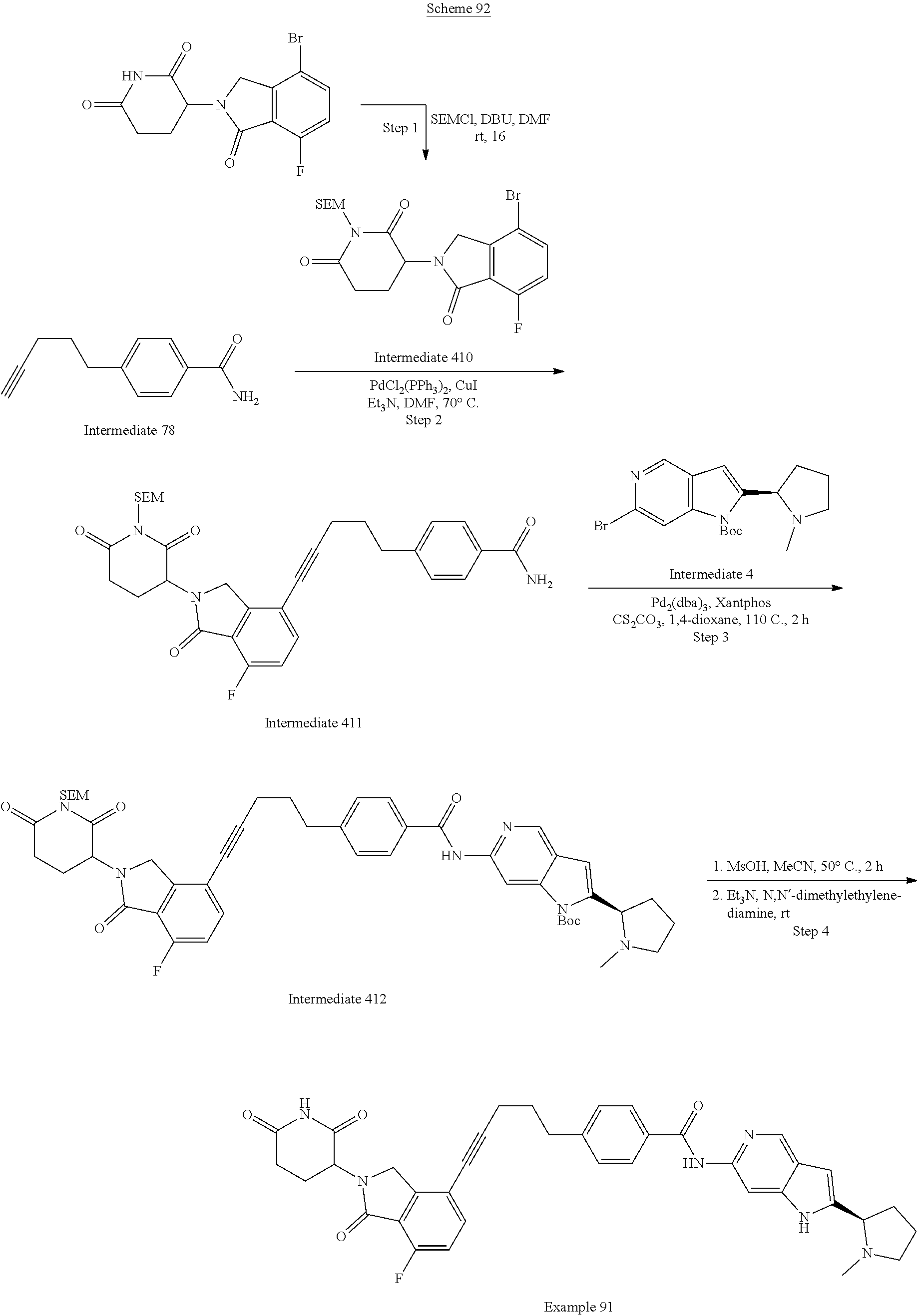

### Step 1

### Intermediate 410: 3-(4-bromo-7-fluoro-1-oxoisoindolin-2-yl)-1-((2-(trimethylsilyl)ethoxy)methyl)piperidine-2,6-dione

To a cooled (0° C.) solution of 3-(4-bromo-7-fluoro-1-oxoisoindolin-2-yl)piperidine-2,6-dione (CAS No. 2438239-34-8, 0.50 g, 1.46 mmol) in dimethylformamide (3 mL) was added 1,8-diazabicyclo[5.4.0]undec-7-ene (0.56 mL, 3.66 mmol) and 2-(trimethylsilyl)ethoxymethyl chloride (0.68 mL, 3.66 mmol) and the reaction mixture was stirred at room temperature for 16 h. The reaction mixture was quenched with water (100 mL) and extracted with ethyl acetate (2×100 mL). The combined organic layers were dried over anhydrous Na₂SO₄ and concentrated in vacuo. The crude material was purified by combi-flash chromatography, by eluting with 50% ethyl acetate in heptane, to afford 3-(4-bromo-7-fluoro-1-oxoisoindolin-2-yl)-1-((2-(trimethylsilyl)ethoxy)methyl)piperidine-2,6-dione (Intermediate 410, 0.40 g, 58%) as a white solid. ¹H NMR (400 MHz, DMSO-d₆) δ ppm −0.02 (s, 9H), 0.81-0.90 (m, 2H), 2.04-2.07 (m, 1H), 2.45-2.49 (m, 1H), 2.76-2.81 (m, 1H), 3.02-3.11 (m, 1H), 3.50-3.55 (m, 2H), 4.20-4.24 (m, 1H), 4.42-4.46 (m, 1H), 5.01-5.09 (m, 2H), 5.21-5.26 (m, 1H), 7.35 (t, J=8.40 Hz, 1H), 7.90 (dd, J=8.40, 3.72 Hz, 1H). Mass spec m/z 469.4 [M−H]⁻.

### Step 2

### Intermediate 411: 4-(5-(2-(2,6-dioxo-1-((2-(trimethylsilyl)ethoxy)methyl)piperidin-3-yl)-7-fluoro-1-oxoisoindolin-4-yl)pent-4-yn-1-yl)benzamide

To a suspension of 4-(pent-4-yn-1-yl)benzamide (Intermediate 78, 0.45 g, 2.41 mmol) and 3-(4-bromo-7-fluoro-1-oxoisoindolin-2-yl)-1-((2-(trimethylsilyl)ethoxy)methyl)piperidine-2,6-dione (Intermediate 410, 0.38 g, 0.80 mmol) in acetonitrile (5 mL) was added triethylamine (3.95 mL, 28.2 mmol). The reaction mixture was purged with argon for 15 min, then copper (I) iodide (0.01 g, 0.08 mmol) and PdCl₂(PPh₃)₂ (0.05 g, 0.08 mmol) were added. The reaction mixture was purged with argon for a further 10 min and then heated at 70° C. for 1 h. The reaction mixture was filtered through a celite bed, and the filter pad was washed with ethyl acetate (100 mL). The filtrate was concentrated in vacuo and the crude material purified by combi-flash chromatography, by eluting with 90% ethyl acetate in heptane, to afford 4-(5-(2-(2,6-dioxo-1-((2-(trimethylsilyl)ethoxy)methyl)piperidin-3-yl)-7-fluoro-1-oxoisoindolin-4-yl)pent-4-yn-1-yl)benzamide (Intermediate 411, 0.35 g, 75%) as a yellow solid. ¹H NMR (400 MHz, DMSO-d₆) δ ppm −0.04 (s, 9H), 0.79-0.84 (m, 2H), 1.86-1.90 (m, 2H), 2.02-2.10 (m, 1H), 2.69-2.73 (m, 1H), 2.75-2.83 (m, 3H), 2.99-3.11 (m, 2H), 3.45-3.58 (m, 2H), 4.00-4.06 (m, 1H), 4.27-4.31 (m, 1H), 4.46-4.53 (m, 1H), 4.99-5.10 (m, 2H), 5.20-5.25 (m, 1H), 7.24 (br s, 1H), 7.30-7.34 (m, 2H), 7.68-7.71 (m, 1H), 7.80 (d, J=8.4 Hz, 2H), 7.82-7.86 (m, 1H), 7.95 (br s, 1H). Mass spec m/z 576.2 [M−H]⁻.

### Step 3

### Intermediate 412: tert-butyl 6-(4-(5-(2-(2,6-dioxo-1-((2-(trimethylsilyl)ethoxy)methyl)piperidin-3-yl)-7-fluoro-1-oxoisoindolin-4-yl)pent-4-yn-1-yl)benzamido)-2-((R)-1-methylpyrrolidin-2-yl)-1H-pyrrolo[3,2-c]pyridine-1-carboxylate

To a solution of 4-(5-(2-(2,6-dioxo-1-((2-(trimethylsilyl)ethoxy)methyl)piperidin-3-yl)-7-fluoro-1-oxoisoindolin-4-yl)pent-4-yn-1-yl)benzamide (Intermediate 411, 0.35 g, 0.60 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.27 g, 0.72 mmol) followed by cesium carbonate (0.59 g, 1.81 mmol). The reaction mixture was purged with argon for 10 min, then Pd₂(dba)₃ (0.08 g, 0.09 mmol) and Xantphos (0.10 g, 0.18 mmol) were added. The reaction mixture was purged with argon for another 10 min and then stirred at 110° C. for 2 h. The reaction mixture was filtered, the filtrate concentrated in vacuo and the crude material was purified by combi-flash chromatography, by eluting with 15% MeOH in DCM, to afford tert-butyl 6-(4-(5-(2-(2,6-dioxo-1-((2-(trimethylsilyl)ethoxy)methyl)piperidin-3-yl)-7-fluoro-1-oxoisoindolin-4-yl)pent-4-yn-1-yl)benzamido)-2-((R)-1-methylpyrrolidin-2-yl)-1H-pyrrolo[3,2-c]pyridine-1-carboxylate (Intermediate 412, 0.30 g, 56%) as a yellow solid. ¹H NMR (400 MHz, DMSO-d₆) δ ppm −0.04 (s, 9H), 0.78-0.87 (m, 2H), 1.56-1.68 (m, 2H), 1.69 (s, 9H), 1.71-1.80 (m, 3H), 1.90-2.10 (m, 4H), 2.35 (s, 3H), 2.36-2.44 (m, 2H), 2.76-2.86 (m, 3H), 3.04-3.16 (m, 2H), 3.47-3.55 (m, 2H), 3.87-3.98 (m, 1H), 4.28-4.33 (m, 1H), 4.46-4.51 (m, 1H), 5.01-5.08 (m, 2H), 5.21-5.27 (m, 1H), 6.75 (s, 1H), 7.28-7.40 (m, 3H), 7.65-7.74 (m, 1H), 7.99 (d, J=8.40 Hz, 2H), 8.59 (s, 1H), 8.92 (s, 1H), 10.59 (br s, 1H). Mass spec m/z 877.4 [M+H]⁺.

### Step 4

### Example 91: 4-(5-(2-(2,6-dioxopiperidin-3-yl)-7-fluoro-1-oxoisoindolin-4-yl)pent-4-yn-1-yl)-N-(2-((R)-1-methylpyrrolidin-2-yl)-1H-pyrrolo[3,2-c]pyridin-6-yl)benzamide

To a solution of tert-butyl 6-(4-(5-(2-(2,6-dioxo-1-((2-(trimethylsilyl)ethoxy)methyl)piperidin-3-yl)-7-fluoro-1-oxoisoindolin-4-yl)pent-4-yn-1-yl)benzamido)-2-((R)-1-methylpyrrolidin-2-yl)-1H-pyrrolo[3,2-c]pyridine-1-carboxylate (Intermediate 412, 0.33 g, 0.37 mmol) in acetonitrile (6 mL) was added methanesulfonic acid (0.24 mL, 3.76 mmol) and the reaction mixture was heated at 50° C. for 2 h. The reaction mixture was cooled to room temperature, then N,N′-dimethylethylenediamine (0.21 mL, 1.88 mmol) followed by triethylamine (1.05 mL, 7.52 mmol) were added and the mixture was stirred at room temperature for 2 h. The reaction mixture was concentrated in vacuo and the resultant residue was diluted with water (10 mL). The resultant precipitate was collected by filtration and dried in vacuo. The crude solid was purified by preparative HPLC (Method A) to afford 4-(5-(2-(2,6-dioxopiperidin-3-yl)-7-fluoro-1-oxoisoindolin-4-yl)pent-4-yn-1-yl)-N-(2-((R)-1-methylpyrrolidin-2-yl)-1H-pyrrolo[3,2-c]pyridin-6-yl)benzamide (Example 91, 0.16 g, 44%) as an off-white solid. ¹H NMR (400 MHz, DMSO-d₆) δ ppm 1.74-1.96 (m, 5H), 1.97-2.06 (m, 1H), 2.09-2.15 (m, 1H), 2.16 (s, 3H), 2.22-2.26 (m, 1H), 2.55-2.63 (m, 1H), 2.82 (t, J=7.63 Hz, 2H), 2.86-2.96 (m, 1H), 3.10-3.18 (m, 1H), 3.29 (d, J=1.50 Hz, 1H), 3.33-3.34 (m, 1H), 4.32-4.40 (m, 1H), 4.45-4.53 (m, 1H), 4.45-4.53 (m, 1H), 4.51-4.53 (m, 1H), 5.08-5.13 (m, 1H), 6.39 (s, 1H), 7.29-7.37 (m, 3H), 7.67-7.72 (m, 1H), 7.99 (d, J=8.40 Hz, 2H), 8.17 (s, 1H), 8.50 (s, 1H), 10.35 (br s, 1H), 11.04 (br s, 1H), 11.36 (br s, 1H). Mass spec m/z 647.0 [M+H]⁺.

### Example 92 was Prepared Following Scheme 93

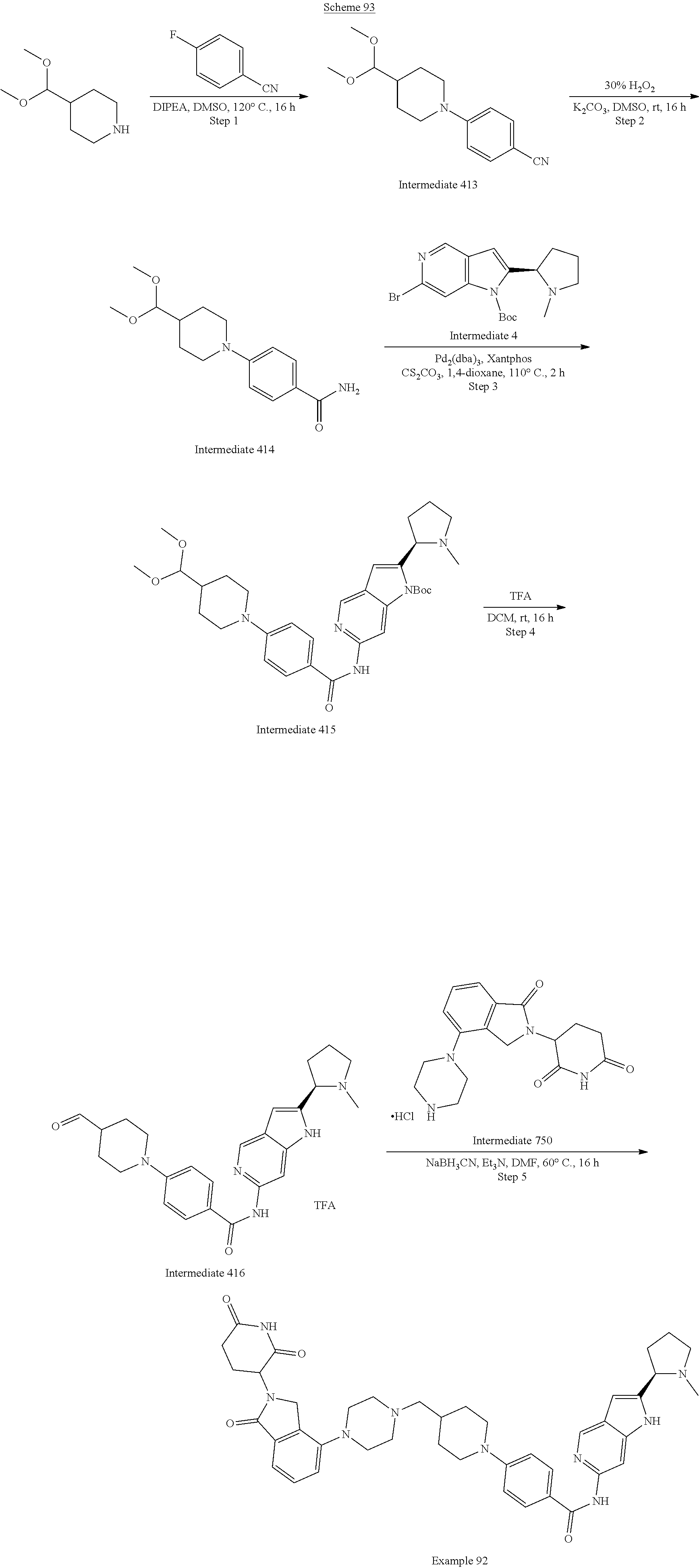

### Step 1

### Intermediate 413: 4-(4-(dimethoxymethyl)piperidin-1-yl)benzonitrile

To a solution of 4-(dimethoxymethyl)piperidine (CAS No. 188646-83-5, 8.67 g, 54.5 mmol) in dimethyl sulfoxide (30 mL) was added 4-fluorobenzonitrile (CAS No. 1194-02-1, 6.0 g, 49.5 mmol) and N,N-diisopropylethylamine (34.6 mL, 198 mmol). The reaction mixture was stirred at 120° C. for 16 h then quenched with ice cold water (100 mL). The resultant precipitate was collected by filtration, washed with n-pentane (50 mL) and dried in vacuo to afford 4-(4-(dimethoxymethyl)piperidin-1-yl)benzonitrile (Intermediate 413, 12.0 g, 93%) as an off-white solid, which was used for the next step without further purification. ¹H NMR (400 MHz, DMSO-d₆) δ ppm 1.18-1.30 (m, 2H), 1.63-1.72 (m, 2H), 1.77-1.89 (m, 1H), 2.78-2.84 (m, 2H), 3.26 (s, 6H), 3.88-3.96 (m, 2H), 4.03-4.09 (m, 1H), 6.98 (d, J=8.61 Hz, 2H), 7.53 (d, J=8.61 Hz, 2H). Mass spec m/z 261.1 [M+H]⁺.

### Step 2

### Intermediate 414: 4-(4-(dimethoxy methyl)piperidin-1-yl)benzamide

To a solution of 4-(4-(dimethoxymethyl)piperidin-1-yl)benzonitrile (Intermediate 413, 4.00 g, 15.4 mmol) in dimethyl sulfoxide (10 mL) was added potassium carbonate (3.22 g, 23.1 mmol) and 30% hydrogen peroxide in water (4.48 g, 46.1 mmol) and the reaction mixture was stirred at room temperature for 16 h. The reaction mixture was quenched with water (100 mL), the resultant precipitate was collected by filtration and dried in vacuo to afford 4-(4-(dimethoxymethyl)piperidin-1-yl)benzamide (Intermediate 414, 3.1 g, 67%) as an off-white solid, which was used for the next step without further purification. ¹H NMR (400 MHz, DMSO-d₆) δ ppm 1.23-1.32 (m, 2H), 1.67-1.78 (m, 3H), 2.67-2.74 (m, 2H), 3.26 (s, 6H), 3.79-3.89 (m, 2H), 4.02-4.09 (m, 1H), 6.89 (d, J=8.61 Hz, 2H), 6.93 (br s, 1H), 7.63 (br s, 1H), 7.71 (d, J=8.61 Hz, 2H). Mass spec m/z 279.1 [M+H]⁺.

### Step 3

### Intermediate 415: tert-butyl (R)-6-(4-(4-(dimethoxymethyl)piperidin-1-yl)benzamido)-2-(1-methylpyrrolidin-2-yl)-1H-pyrrolo[3,2-c]pyridine-1-carboxylate

To a solution of 4-(4-(dimethoxymethyl)piperidin-1-yl)benzamide (Intermediate 414, 1.0 g, 3.59 mmol) in 1,4-dioxane (20 mL) was added tert-butyl (R)-6-bromo-2-(1-methylpyrrolidin-2-yl)-1H-pyrrolo[3,2-c]pyridine-1-carboxylate (Intermediate 4, 1.53 g, 3.95 mmol) followed by cesium carbonate (3.57 g, 10.8 mmol). The reaction mixture was purged with argon for 15 min then Pd₂(dba)₃ (0.50 g, 0.53 mmol) and Xantphos (0.64 g, 1.07 mmol) were added. The reaction mixture was again purged with argon for 10 min and stirred at 100° C. for 2 h then concentrated in vacuo. The crude material was purified by combi-flash chromatography, by eluting with 11% MeOH in DCM, to afford tert-butyl (R)-6-(4-(4-(dimethoxymethyl)piperidin-1-yl)benzamido)-2-(1-methylpyrrolidin-2-yl)-1H-pyrrolo[3,2-c]pyridine-1-carboxylate (Intermediate 415, 1.7 g, 82%) as an off-white solid. ¹H NMR (400 MHz, DMSO-d₆) δ ppm 1.22-1.38 (m, 3H), 1.62-1.67 (m, 1H), 1.70 (s, 9H), 1.72-1.83 (m, 8H), 2.35 (s, 3H), 2.38-2.43 (m, 2H), 3.10-3.17 (m, 1H), 3.28 (s, 6H), 3.91-3.93 (m, 1H), 4.07-4.09 (m, 1H), 6.74 (s, 1H), 6.96 (d, J=8.61 Hz, 2H), 7.95 (d, J=8.61 Hz, 2H), 8.57 (s, 1H), 8.92 (s, 1H), 10.29 (br s, 1H). Mass spec m/z 578.3 [M+H]⁺.

### Step 4

### Intermediate 416: (R)-4-(4-formylpiperidin-1-yl)-N-(2-(1-methylpyrrolidin-2-yl)-1H-pyrrolo[3,2-c]pyridin-6-yl)benzamide trifluoroacetate

To a cooled (0° C.) solution of tert-butyl (R)-6-(4-(4-(dimethoxymethyl)piperidin-1-yl)benzamido)-2-(1-methylpyrrolidin-2-yl)-1H-pyrrolo[3,2-c]pyridine-1-carboxylate (Intermediate 415, 1.0 g, 1.73 mmol) in dichloromethane (10 mL) was added trifluoroacetic acid (2.01 mL, 26.0 mmol) and the reaction mixture was stirred at room temperature for 16 h. The reaction mixture was concentrated in vacuo to afford (R)-4-(4-formylpiperidin-1-yl)-N-(2-(1-methylpyrrolidin-2-yl)-1H-pyrrolo[3,2-c]pyridin-6-yl)benzamide trifluoroacetate (Intermediate 416, 1.1 g) as pale-yellow oil, which was used for next step without purification. ¹H NMR (400 MHz, DMSO-d₆) δ ppm 1.09-1.11 (m, 1H), 1.55-1.65 (m, 2H), 1.93-1.97 (m, 2H), 2.13-2.27 (m, 2H), 2.31 (s, 3H), 2.84-2.91 (m, 2H), 3.06-3.12 (m, 1H), 3.24-3.43 (m, 2H), 3.70-3.82 (m, 1H), 3.84-3.93 (m, 2H), 4.66-4.76 (m, 1H), 7.10 (d, J=8.22 Hz, 2H), 7.18 (s, 1H), 7.85-8.03 (m, 3H), 9.01 (s, 1H), 9.65 (s, 1H), 10.36 (br s, 1H), 11.28 (br s, 1H), 12.85 (br s, 1H). Mass spec m/z 432.1 [M+H]⁺.

### Step 4

### Example 92: 4-(4-((4-(2-(2,6-dioxopiperidin-3-yl)-1-oxoisoindolin-4-yl)piperazin-1-yl)methyl)piperidin-1-yl)-N-(2-((R)-1-methylpyrrolidin-2-yl)-1H-pyrrolo[3,2-c]pyridin-6-yl)benzamide

To a solution of (R)-4-(4-formylpiperidin-1-yl)-N-(2-(1-methylpyrrolidin-2-yl)-1H-pyrrolo[3,2-c]pyridin-6-yl)benzamide trifluoroacetate (Intermediate 416, 0.90 g, 1.65 mmol) in triethylamine (0.92 mL, 6.59 mmol) was added 3-(1-oxo-4-(piperazin-1-yl)isoindolin-2-yl)piperidine-2,6-dione hydrochloride (Intermediate 750, 0.66 g, 1.81 mmol) and sodium cyanoborohydride (0.32 g, 4.94 mmol) in dimethylformamide (10 mL). The reaction mixture was stirred at 60° C. for 16 h. The reaction mixture was concentrated in vacuo, water (50 mL) was added to the residue and the resultant precipitate was collected by filtration and dried in vacuo. The crude material was purified by preparative HPLC (Method A) to afford 4-(4-((4-(2-(2,6-dioxopiperidin-3-yl)-1-oxoisoindolin-4-yl)piperazin-1-yl)methyl)piperidin-1-yl)-N-(2-((R)-1-methylpyrrolidin-2-yl)-1H-pyrrolo[3,2-c]pyridin-6-yl)benzamide (Example 92, 0.16 g, 14%) as an off-white solid. ¹H NMR (400 MHz, DMSO-d₆) δ ppm 1.19-1.21 (m, 2H), 1.75-1.94 (m, 6H), 1.95-2.04 (m, 1H), 2.10-2.15 (m, 1H), 2.17 (s, 3H), 2.17-2.24 (m, 3H), 2.44-2.47 (m, 1H), 2.53-2.67 (m, 6H), 2.77-2.93 (m, 3H), 3.03-3.16 (m, 5H), 3.88-3.92 (m, 2H), 4.26-4.35 (m, 1H), 4.41-4.50 (m, 1H), 5.08-5.13 (m, 1H), 6.37 (s, 1H), 6.97 (d, J=8.94 Hz, 2H), 7.17 (d, J=8.00 Hz, 1H), 7.32 (d, J=7.38 Hz, 1H), 7.41-7.47 (m, 1H), 7.94 (d, J=8.94 Hz, 2H), 8.17 (s, 1H), 8.48 (s, 1H), 10.04 (br s, 1H), 10.97 (br s, 1H), 11.31 (br s, 1H). Mass spec m/z 744.2 [M+H]⁺.

### Example 93 was Prepared Following Scheme 94

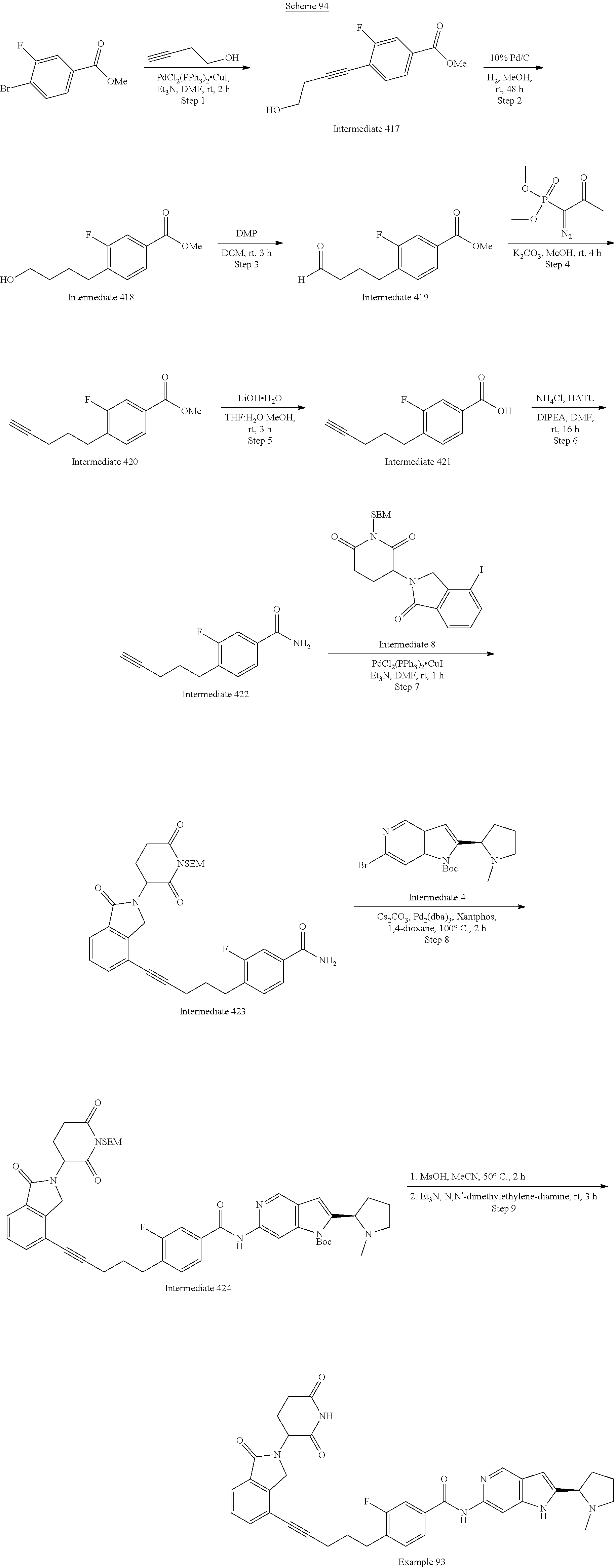

### Step 1

### Intermediate 417: methyl 3-fluoro-4-(4-hydroxybut-1-yn-1-yl)benzoate

To a solution of methyl 4-bromo-3-fluorobenzoate (CAS No. 849758-12-9, 10.00 g, 42.91 mmol) in dimethylformamide (10 mL) was added but-3-yn-1-ol (CAS No. 927-74-2, 3.01 g, 42.91 mmol) followed by triethylamine (156.31 g, 1502 mmol). The reaction mixture was purged with argon for 15 min then copper (I) iodide (0.82 g, 4.29 mmol) and PdCl₂(PPh₃)₂ (3.01 g, 4.29 mmol) were added. The reaction mixture was further purged with argon for 10 min and stirred at room temperature for 2 h. After completion, the reaction mixture was diluted with water (100 mL) and extracted with ethyl acetate (3×100 mL). The combined organic layers were dried over anhydrous Na₂SO₄, and concentrated in vacuo. The crude material was purified by combi-flash chromatography, by eluting with 50% ethyl acetate in heptane, to afford methyl 3-fluoro-4-(4-hydroxybut-1-yn-1-yl)benzoate (Intermediate 417, 8.0 g, 84%) as a brown solid. ¹H NMR (400 MHz, DMSO-d₆) δ ppm 2.64 (t, J=6.80 Hz, 2H), 3.58-3.63 (m, 2H), 3.86 (s, 3H), 4.95 (t, J=5.60 Hz, 1H), 7.62 (t, J=7.60 Hz, 1H), 7.37-7,39 (m, 2H). Mass spec m/z 223.1 [M+H]⁺.

### Step 2

### Intermediate 418: methyl 3-fluoro-4-(4-hydroxybutyl)benzoate

To a solution of methyl 3-fluoro-4-(4-hydroxybut-1-yn-1-yl)benzoate (Intermediate 417, 8.0 g, 36.04 mmol) in MeOH was added 10% Pd/C (4.0 g) and the reaction mixture was stirred for 48 h under H₂ atmosphere (100 psi) at room temperature for 16 h. After completion, the reaction mixture was filtered through a celite bed and the filtrate was concentrated in vacuo to afford methyl 3-fluoro-4-(4-hydroxybutyl)benzoate (Intermediate 418, 6.20 g, 77%) as a light yellow solid that was carried forward into next step without purification. Mass spec m/z 227.1 [M+H]⁺.

### Step 3

### Intermediate 419: methyl 3-fluoro-4-(4-oxobutyl)benzoate

To a cooled (0° C.) solution of methyl 3-fluoro-4-(4-hydroxybutyl)benzoate (Intermediate 418, 6.00 g, 26.52 mmol) in dichloromethane (90 mL) was added Dess-Martin periodinane (7.64 g, 39.78 mmol) and the reaction mixture was stirred at room temperature for 3 h. After completion, the reaction mixture was diluted with saturated aqueous sodium bicarbonate solution (100 mL) and saturated aqueous sodium thiosulphate solution (100 mL) and extracted with dichloromethane (2×100 mL). The combined organic layers were dried over Na₂SO₄, filtered, and concentrated in vacuo to afford methyl 3-fluoro-4-(4-oxobutyl)benzoate (Intermediate 419, 3.41 g, 57%) as a yellow oil. ¹H NMR (400 MHz, DMSO-d₆) δ ppm 1.82-1.86 (m, 2H), 2.47-2.51 (m, 2H), 2.69 (t, J=8.0 Hz, 2H), 3.85 (s, 3H), 7.47 (t, J=7.60 Hz, 1H), 7.63-7.66 (m, 1H), 7.73-7.76 (m, 1H), 9.66 (s, 1H).

### Step 4

### Intermediate 420: methyl 3-fluoro-4-(pent-4-yn-1-yl)benzoate

To a solution of methyl 3-fluoro-4-(4-oxobutyl)benzoate (Intermediate 419, 3.40 g, 15.0 mmol) in methanol (30 mL) was added potassium carbonate (6.90 g, 30 mmol) followed by dimethyl (1-diazo-2-oxopropyl)phosphonate (CAS No. 90965-06-3, 3.26 g, 17 mmol). The reaction mixture was stirred at room temperature for 4 h. After completion, the reaction mixture was diluted with water (100 mL) and extracted with diethyl ether (3×200 mL). The combined organic layers were dried over anhydrous Na₂SO₄, filtered, and concentrated in vacuo. The crude material was purified by combi-flash chromatography, by eluting with 5-10% ethyl acetate in heptane, to afford methyl 3-fluoro-4-(pent-4-yn-1-yl)benzoate (Intermediate 420, 1.5 g, 45%) as a colorless oil. ¹H NMR (400 MHz, DMSO-d₆) δ ppm 1.74-1.77 (m, 2H), 2.18-2.22 (m, 2H), 2.77 (t, J=8.0 Hz, 2H), 2.85 (t, J=2.80 Hz, 1H), 3.86 (s, 3H), 7.45 (t, J=8.0 Hz, 1H), 7.66 (dd, J=1.60, 10.40 Hz, 1H), 7.74 (dd, J=1.60, 8.0 Hz, 1H). Mass spec m/z 221.0 [M+H]⁺.

### Step 5

### Intermediate 421: 3-fluoro-4-(pent-4-yn-1-yl)benzoic acid

To a solution of methyl 3-fluoro-4-(pent-4-yn-1-yl)benzoate (Intermediate 420, 1.45 g, 6.58 mmol) in tetrahydrofuran (5 mL), methanol (5 mL) and water (5 mL) was added lithium hydroxide hydrate (1.10 g, 26.30 mmol) and the reaction mixture was stirred at room temperature for 3 h. After completion, the reaction mixture was concentrated in vacuo. The reaction mixture was diluted with water (20 mL) and the aqueous layer was acidified by adjusting to pH~2 by the addition of 1N aqueous HCl solution. The resulting solid precipitate was collected by filtration, washed with water (50 mL) and dried in vacuo to afford 3-fluoro-4-(pent-4-yn-1-yl)benzoic acid (Intermediate 421, 1.35 g) as an off-white solid, which was used for the next step without further purification. ¹H NMR (400 MHz, DMSO-d₆) δ ppm 1.71-1.78 (m, 2H), 2.18-2.22 (m, 2H), 2.76 (t, J=8.0 Hz, 2H), 2.83 (t, J=2.40 Hz, 1H), 7.43 (t, J=7.60 Hz, 1H), 7.60 (dd, J=1.60, 10.40 Hz, 1H), 7.71 (dd, J=7.60, 1.60 Hz, 1H), 13.51 (br s, 1H). Mass spec m/z 205.0 [M−H]⁻.

### Step 6

### Intermediate 422: 3-fluoro-4-(pent-4-yn-1-yl)benzamide

To a solution of 3-fluoro-4-(pent-4-yn-1-yl)benzoic acid (Intermediate 421, 1.35 g, 6.55 mmol) in dimethylformamide (15 mL) were added HATU (3.73 g, 9.82 mmol) and N,N-diisopropylethylamine (4.56 mL, 26.2 mmol) and the reaction mixture was stirred at room temperature for 20 min. Ammonium chloride (1.75 g, 32.70 mmol) was then added, and the reaction mixture was stirred at room temperature for 16 h. After completion, the reaction mixture was quenched with ice cold water (100 mL), the resulting precipitate was collected by filtration, washed with water (50 mL) and dried in vacuo to afford 3-fluoro-4-(pent-4-yn-1-yl)benzamide (Intermediate 422, 1.20 g) as a white solid. ¹H NMR (400 MHz, DMSO-d₆) δ ppm 1.72-1.77 (m, 2H), 2.17-2.22 (m, 2H), 2.73 (t, J=7.60 Hz, 2H), 2.83 (t, J=2.80 Hz, 1H), 7.37 (t, J=8.0 Hz, 1H), 7.44 (br s, 1H), 7.61-7.66 (m, 2H), 7.99 (br s, 1H). Mass spec m/z 206.1 [M+H]⁺.

### Step 7

### Intermediate 423: 4-(5-(2-(2,6-dioxo-1-((2-(trimethylsilyl)ethoxy)methyl)piperidin-3-yl)-1-oxoisoindolin-4-yl)pent-4-yn-1-yl)-3-fluorobenzamide

To a solution of 3-fluoro-4-(pent-4-yn-1-yl)benzamide (Intermediate 422, 0.27 g, 1.32 mmol) in dimethylformamide (1.0 mL) was added 3-(4-iodo-1-oxoisoindolin-2-yl)-1-((2-(trimethylsilyl)ethoxy)methyl)piperidine-2,6-dione (Intermediate 8, 0.73 g, 1.45 mmol) followed by triethylamine (6.84 mL, 48.68 mmol). The reaction mixture was purged with argon for 15 min, then copper (I) iodide (0.036 g, 0.19 mmol) and PdCl₂(PPh₃)₂ (0.091 g, 0.13 mmol) were added. The reaction mixture was purged with argon for further 10 min and stirred at room temperature for 1 h. After completion, the reaction mixture was diluted with water (40 mL) and extracted with ethyl acetate (3×50 mL). The combined organic layers were dried over anhydrous Na₂SO₄, and concentrated in vacuo. The crude material was purified by combi-flash chromatography, by eluting with 80% ethyl acetate in heptane, to afford 4-(5-(2-(2,6-dioxo-1-((2-(trimethylsilyl)ethoxy)methyl)piperidin-3-yl)-1-oxoisoindolin-4-yl)pent-4-yn-1-yl)-3-fluorobenzamide (Intermediate 423, 0.5 g, 66%) as a yellow solid. ¹H NMR (400 MHz, DMSO-d₆) δ ppm −0.05 (s, 9H), 0.78-0.87 (m, 2H), 1.86-1.89 (m, 2H), 1.99-2.22 (m, 1H), 2.36-2.43 (m, 2H), 2.77-2.82 (m, 3H), 3.03-3.10 (m, 1H), 3.49-3.54 (m, 2H), 4.07-4.11 (m, 1H), 4.26-4.30 (m, 1H), 4.47-4.52 (m, 1H), 5.01.5.09 (m, 2H), 5.25-5.30 (m, 1H), 7.40 (d, J=8.0 Hz, 1H), 7.44 (br s, 1H), 7.53 (t, J=8.0 Hz, 1H), 7.61-7.67 (m, 3H), 7.73 (d, J=7.20 Hz, 1H), 7.95-7.98 (m, 1H).

### Step 8

### Intermediate 424: tert-butyl 6-(4-(5-(2-(2,6-dioxo-1-((2-(trimethylsilyl)ethoxy)methyl)piperidin-3-yl)-1-oxoisoindolin-4-yl)pent-4-yn-1-yl)-3-fluorobenzamido)-2-((R)-1-methylpyrrolidin-2-yl)-1H-pyrrolo[3,2-c]pyridine-1-carboxylate

To a solution of 4-(5-(2-(2,6-dioxo-1-((2-(trimethylsilyl)ethoxy)methyl)piperidin-3-yl)-1-oxoisoindolin-4-yl)pent-4-yn-1-yl)-3-fluorobenzamide (Intermediate 423, 0.45 g, 0.78 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.33 g, 0.86 mmol) followed by cesium carbonate (0.76 g, 2.34 mmol). The reaction mixture was purged with argon for 15 min then Pd₂(dba)₃ (0.10 g, 0.12 mmol) and Xantphos (0.13 g, 0.23 mmol) were added. The reaction mixture was purged with argon for another 10 min and stirred at 100° C. for 2 h. After completion, the reaction mixture was filtered through a celite bed, the filter pad was washed with dichloromethane (20 mL) and the filtrate was concentrated in vacuo. The crude material was purified by combi-flash chromatography, by eluting with 5% MeOH in DCM, to afford tert-butyl 6-(4-(5-(2-(2,6-dioxo-1-((2-(trimethylsilyl)ethoxy)methyl)piperidin-3-yl)-1-oxoisoindolin-4-yl)pent-4-yn-1-yl)-3-fluorobenzamido)-2-((R)-1-methylpyrrolidin-2-yl)-1H-pyrrolo[3,2-c]pyridine-1-carboxylate (Intermediate 424, 0.41 g, 60%) as a yellow solid. ¹H NMR (400 MHz, DMSO-d₆) δ ppm −0.06 (s, 9H), 0.77-0.84 (m, 2H), 1.61-1.64 (m, 2H), 1.70 (s, 9H), 1.74-1.76 (m, 4H), 1.89-1.92 (m, 2H), 2.01-2.09 (m, 1H), 2.35 (s, 3H), 2.37-2.40 (m, 2H), 2.82-2.86 (m, 3H), 3.04-3.12 (m, 2H), 3.46-3.54 (m, 2H), 3.90-3.92 (m, 1H), 4.26-4.31 (m, 1H), 4.48-4.53 (m, 1H), 5.01-5.09 (m, 2H), 5.27-5.29 (m, 1H), 6.76 (s, 1H), 7.54 (t, J=7.60 Hz, 1H), 7.45 (m, 2H), 7.65 (d, J=7.60 Hz, 1H), 7.73 (d, J=7.60 Hz, 1H), 7.83-7.87 (m, 2H), 8.60 (s, 1H), 8.91 (s, 1H). Mass spec m/z 877.1 [M+H]⁺.

### Step 9

### Example 93: 4-(5-(2-(2,6-dioxopiperidin-3-yl)-1-oxoisoindolin-4-yl)pent-4-yn-1-yl)-3-fluoro-N-(2-((R)-1-methylpyrrolidin-2-yl)-1H-pyrrolo[3,2-c]pyridin-6-yl)benzamide

To a solution of tert-butyl 6-(4-(5-(2-(2,6-dioxo-1-((2-(trimethylsilyl)ethoxy)methyl)piperidin-3-yl)-1-oxoisoindolin-4-yl)pent-4-yn-1-yl)-3-fluorobenzamido)-2-((R)-1-methylpyrrolidin-2-yl)-1H-pyrrolo[3,2-c]pyridine-1-carboxylate (Intermediate 424, 0.40 g, 0.46 mmol) in acetonitrile (8.0 mL) was added methanesulfonic acid (0.44 g, 4.6 mmol) and the reaction mixture was stirred at 50° C. for 2 h. After cooling to room temperature, N,N′-dimethylethylenediamine (0.20 g, 2.28 mmol) followed by triethylamine (0.92 g, 9.12 mmol) were added and the reaction mixture was stirred at room temperature for 3 h. After completion, the reaction mixture was concentrated in vacuo. The obtained residue was diluted with cold water (50 mL), the resulting solid precipitate was collected by filtration and dried in vacuo. The crude material was purified by preparative HPLC (Method A) to afford 4-(5-(2-(2,6-dioxopiperidin-3-yl)-1-oxoisoindolin-4-yl)pent-4-yn-1-yl)-3-fluoro-N-(2-((R)-1-methylpyrrolidin-2-yl)-1H-pyrrolo[3,2-c]pyridin-6-yl)benzamide (Example 93, 0.058 g, 20%) as an off-white solid. ¹H NMR (400 MHz, DMSO-d₆) δ ppm 1.83-1.93 (m, 5H), 2.01-2.04 (m, 1H), 2.10-2.14 (m, 1H), 2.17 (s, 3H), 2.23-2.27 (m, 1H), 2.40-2.43 (m, 1H), 2.52-2.57 (m, 3H), 2.84-2.90 (m, 3H), 3.12-3.16 (m, 1H), 3.32-3.34 (m, 1H), 4.33-4.37 (m, 1H), 4.47-4.51 (m, 1H), 5.13-5.17 (m, 1H), 6.40 (s, 1H), 7.46-7.55 (m, 2H), 7.66 (d, J=7.60 Hz, 1H), 7.69 (d, J=7.60 Hz, 1H), 7.83-7.88 (m, 2H), 8.18 (s, 1H), 8.51 (s, 1H), 10.51 (s, 1H), 11.02 (s 1H), 11.38 (s, 1H). ¹⁹F NMR (400 MHz, DMSO-d₆) δ ppm −119.58. Mass spec m/z 647.0 [M+H]⁺.

### Example 94 was Prepared Following Scheme 95

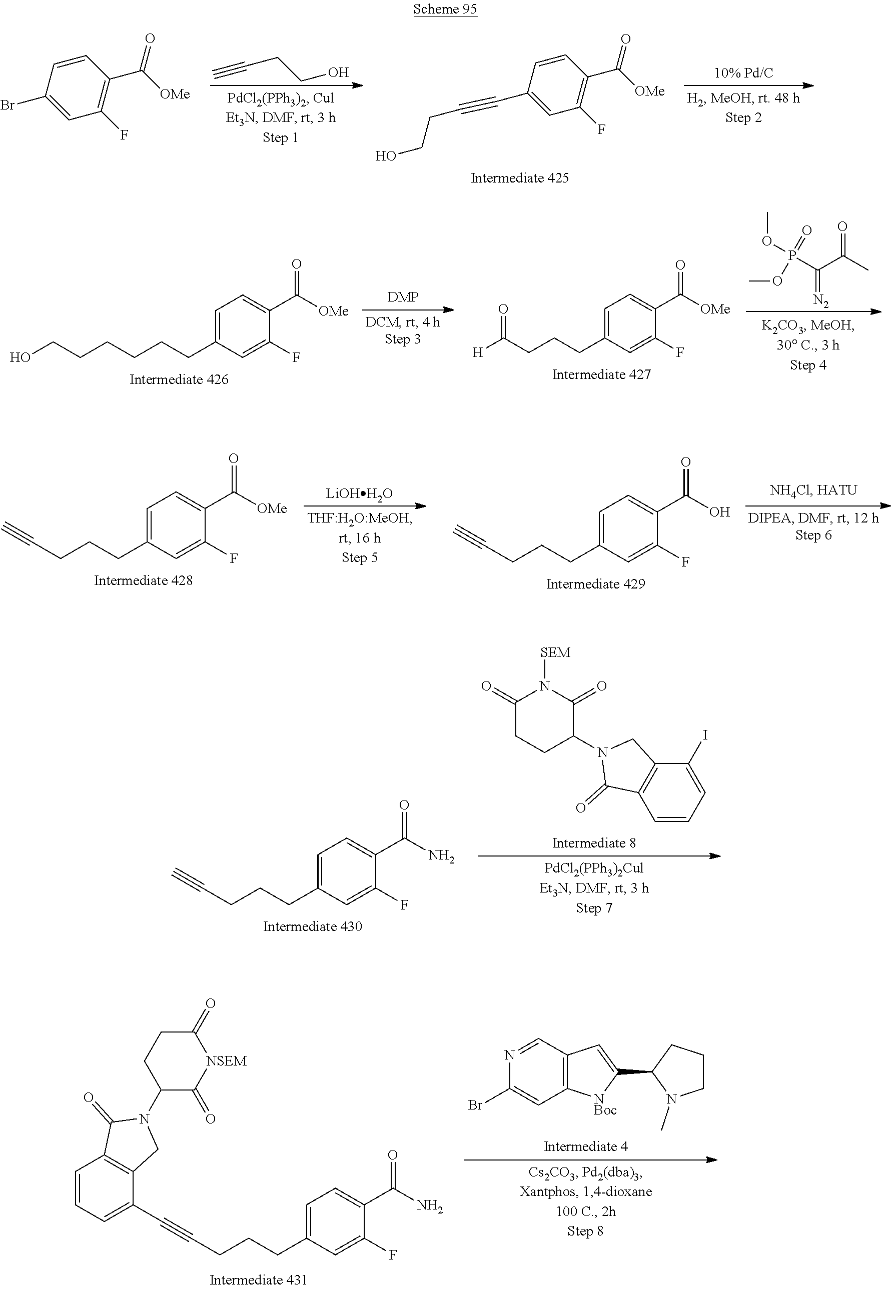

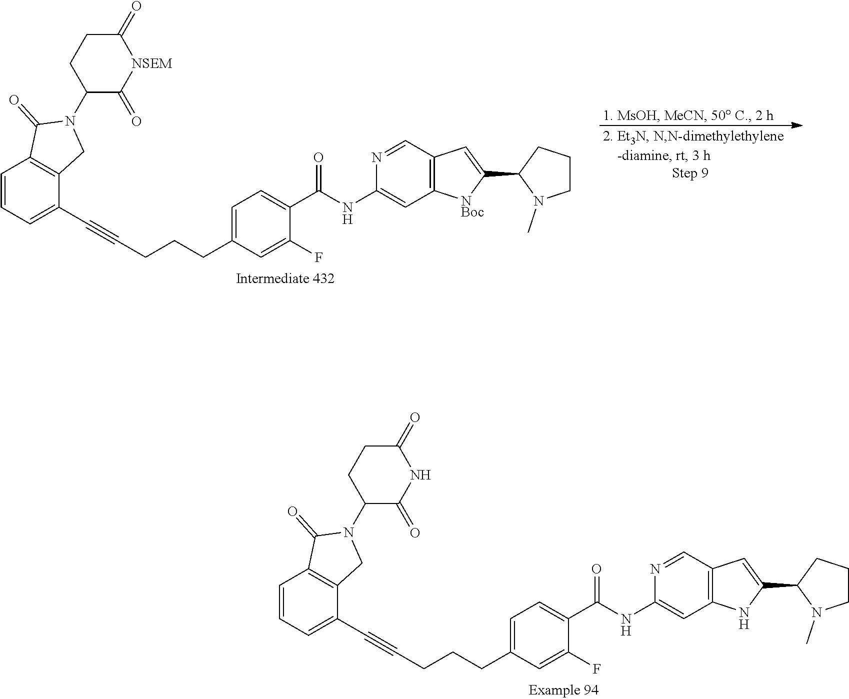 [SMILES: CN1CCC[C@@H]1c1cc2cnc(NC(=O)c3ccc(CCCC#Cc4cccc5c4CN(C4CCC(=O)N(COCC[Si](C)(C)C)C4=O)C5=O)cc3F)cc2n1C(=O)OC(C)(C)C ; CN1CCC[C@@H]1c1cc2cnc(NC(=O)c3ccc(CCCC#Cc4cccc5c4CN(C4CCC(=O)NC4=O)C5=O)cc3F)cc2[nH]1]

### Step 1

### Intermediate 425: methyl 2-fluoro-4-(4-hydroxybut-1-yn-1-yl)benzoate

To a solution of methyl 4-bromo-2-fluorobenzoate (CAS No. 179232-29-2, 10.00 g, 42.93 mmol) in dimethylformamide (20 mL) was added but-3-yn-1-ol (CAS No. 927-74-2, 4.50 g, 64.37 mmol) followed by triethylamine (157 g, 1544 mmol). The reaction mixture was purged with argon for 15 min then copper (I) iodide (0.84 g, 4.29 mmol) and PdCl₂(PPh₃)₂ (3.40 g, 4.29 mmol) were added. The reaction mixture was further purged with argon for 10 min and stirred at room temperature for 3 h. After completion, the reaction mixture was diluted with water (200 mL) and extracted with ethyl acetate (3×200 mL). The combined organic layers were dried over anhydrous Na₂SO₄, and concentrated in vacuo. The crude material was purified by combi-flash chromatography, by eluting with 90% ethyl acetate in heptane, to afford methyl 2-fluoro-4-(4-hydroxybut-1-yn-1-yl)benzoate (Intermediate 425, 9.0 g, 94%) as a yellow solid. ¹H NMR (400 MHz, DMSO-d₆) δ ppm 2.59 (t, J=6.40 Hz, 2H), 3.59 (t, J=6.40 Hz, 2H), 3.85 (s, 3H), 4.92 (br s, 1H), 7.32 (m, J=8.0 Hz, 1H), 7.61-7.64 (m, 1H), 7.84 (d, J=8.0 Hz, 1H). MS (ESI) m/z [M−H]⁻ 221.1

### Step 2

### Intermediate 426: methyl 2-fluoro-4-(4-hydroxybutyl)benzoate

To a solution of methyl 2-fluoro-4-(4-hydroxybut-1-yn-1-yl)benzoate (Intermediate 425, 9.0 g, 40.50 mmol) in MeOH (150 ml) was added 10% Pd/C (3.5 g) and the reaction mixture was stirred at room temperature for 48 h under H₂ atmosphere (100 psi). After completion of the reaction, the reaction mixture was filtered through a celite bed and the filtrate was concentrated in vacuo to afford methyl 2-fluoro-4-(4-hydroxybutyl)benzoate (Intermediate 426, 8.90 g, 97%) as a light yellow solid. ¹H NMR (400 MHz, DMSO-d₆) δ ppm 1.39-1.43 (m, 2H), 1.60-1.63 (m, 2H), 2.64 (t, J=7.60 Hz, 2H), 3.38-3.40 (m, 2H), 3.82 (s, 3H), 4.38 (t, J=5.20 Hz, 1H), 7.14-7.20 (m, 1H), 7.60-7.63 (m, 1H), 7.78 (t, J=8.0 Hz, 1H).

### Step 3

### Intermediate 427: methyl 2-fluoro-4-(4-oxobutyl)benzoate

To a cooled (0° C.) solution of methyl 2-fluoro-4-(4-hydroxybutyl)benzoate (Intermediate 426, 8.90 g, 39.34 mmol) in dichloromethane (100 mL) was added Dess-Martin periodinane (26.0 g, 60 mmol) and the reaction mixture was stirred at room temperature for 4 h. After completion, the reaction mixture was diluted with saturated aqueous sodium bicarbonate solution (100 mL) and saturated aqueous sodium thiosulphate solution (200 mL), and extracted with dichloromethane (3×200 mL). The combined organic layers were dried over Na₂SO₄, filtered, and concentrated in vacuo to afford methyl 2-fluoro-4-(4-oxobutyl)benzoate (Intermediate 427, 5.60 g, 63%) as a yellow solid which was used into next step without purification. ¹H NMR (400 MHz, DMSO-d₆) δ ppm 1.81-1.88 (m, 2H), 2.43-2.47 (m, 2H), 2.66 (t, J=7.20 Hz, 2H), 3.83 (s, 3H), 7.16-7.22 (m, 2H), 7.81 (t, J=8.0 Hz, 1H), 9.66 (s, 1H).

### Step 4

### Intermediate 428: methyl 2-fluoro-4-(pent-4-yn-1-yl)benzoate

To a solution of methyl 2-fluoro-4-(4-oxobutyl)benzoate (Intermediate 427: 5.60 g, 25 mmol) in methanol (60 mL) was added potassium carbonate (6.90 g, 50 mmol) followed by dimethyl (1-diazo-2-oxopropyl)phosphonate (CAS No. 90965-06-3, 7.20 g, 37 mmol). The reaction mixture was stirred at 30° C. for 3 h. After completion, the reaction mixture was diluted with water (100 mL) and extracted with diethyl ether (3×100 mL). The combined organic layers were dried over anhydrous Na₂SO₄, filtered, and concentrated in vacuo. The crude material was purified by combi-flash chromatography, by eluting with 0-10% ethyl acetate in heptane, to afford methyl 2-fluoro-4-(pent-4-yn-1-yl)benzoate (Intermediate 428, 3.80 g, 69%) as a yellow oil. ¹H NMR (400 MHz, DMSO-d₆) δ ppm 1.75-1.79 (m, 2H), 2.15-2.19 (m, 2H), 2.74 (t, J=7.60 Hz, 2H), 2.84 (t, J=2.40 Hz, 1H), 3.84 (s, 3H), 7.17-7.23 (m, 2H), 7.81 (t, J=8.0 Hz, 1H). Mass spec m/z 221.3 [M+H]⁺.

### Step 5

### Intermediate 429: 2-fluoro-4-(pent-4-yn-1-yl)benzoic acid

To a solution of methyl 2-fluoro-4-(pent-4-yn-1-yl)benzoate (Intermediate 428, 3.80 g, 17.0 mmol) in tetrahydrofuran (10 mL), methanol (10 mL) and water (10 mL) was added lithium hydroxide hydrate (2.89 g, 69.0 mmol) and the reaction mixture was stirred at room temperature for 16 h. After completion, the reaction mixture was concentrated in vacuo. The resulting residue was diluted with water (20 mL) and then acidified to pH~4 with 1N aqueous HCl solution. The resultant precipitate was collected by filtration, washed with water (30 mL), and dried in vacuo to afford 2-fluoro-4-(pent-4-yn-1-yl)benzoic acid (Intermediate 429, 3.40 g, 96%) as an off-white solid, which was used for the next step without further purification. ¹H NMR (400 MHz, DMSO-d₆) δ ppm 1.69-1.74 (m, 2H), 2.23 (t, J=7.20 Hz, 2H), 2.77 (t, J=7.20 Hz, 2H), 2.82 (s, 1H), 7.52-7.54 (m, 3H), 13.50 (br s, 1H). Mass spec m/z 205.3 [M−H]⁻.

### Step 6

### Intermediate 430: 2-fluoro-4-(pent-4-yn-1-yl)benzamide

To a solution of 2-fluoro-4-(pent-4-yn-1-yl)benzoic acid (Intermediate 429, 3.40 g, 16.0 mmol) in dimethylformamide (40 mL) were added HATU (9.50 g, 25.0 mmol) and N,N-diisopropylethylamine (8.55 mL, 49.0 mmol) and the reaction mixture was stirred at room temperature for 20 min. Ammonium chloride (3.53 g, 66.0 mmol) was then added and the reaction mixture was stirred at room temperature for 12 h. After completion, the reaction mixture was quenched with ice cold water (100 mL) and extracted with ethyl acetate (3×100 mL). The combined organic layers were dried over anhydrous Na₂SO₄, filtered, and concentrated in vacuo. The crude residue was purified by combi-flash chromatography, by eluting with 80% ethyl acetate in heptane, to afford 2-fluoro-4-(pent-4-yn-1-yl)benzamide (Intermediate 430, 3.0 g, 67%) as an off-white solid. ¹H NMR (400 MHz, DMSO-d₆) δ ppm 1.72-1.79 (m, 2H), 2.16 (t, J=7.60 Hz, 2H), 2.70 (t, J=7.60 Hz, 2H), 2.82 (s, 1H), 7.09-7.11 (m, 1H), 7.13 (br s, 1H), 7.56-7.60 (m, 2H), 7.61 (br s, 1H). Mass spec m/z 206.4 [M+H]⁺.

### Step 7

### Intermediate 431: 4-(5-(2-(2,6-dioxo-1-((2-(trimethylsilyl)ethoxy)methyl)piperidin-3-yl)-1-oxoisoindolin-4-yl)pent-4-yn-1-yl)-2-fluorobenzamide

To a solution of 2-fluoro-4-(pent-4-yn-1-yl)benzamide (Intermediate 430, 0.50 g, 2.44 mmol) in dimethylformamide (2 mL) was added 3-(4-iodo-1-oxoisoindolin-2-yl)-1-((2-(trimethylsilyl)ethoxy)methyl)piperidine-2,6-dione (Intermediate 8, 1.22 g, 2.44 mmol) followed by triethylamine (8.92 g, 87.71 mmol). The reaction mixture was purged with argon for 15 min then copper (I) iodide (0.05 g, 0.24 mmol) and PdCl₂(PPh₃)₂ (0.17 g, 0.24 mmol) were added. The reaction mixture was further purged with argon for 10 min and stirred at room temperature for 3 h. After completion, the reaction mixture was diluted with water (100 mL) and extracted with ethyl acetate (3×100 mL). The combined organic layers were dried over anhydrous Na₂SO₄, and concentrated in vacuo. The crude material was purified by combi-flash chromatography, by eluting with 50% ethyl acetate in heptane, to afford 4-(5-(2-(2,6-dioxo-1-((2-(trimethylsilyl)ethoxy)methyl)piperidin-3-yl)-1-oxoisoindolin-4-yl)pent-4-yn-1-yl)-2-fluorobenzamide (Intermediate 431, 0.38 g, 27%) as a yellow solid. The desired product was confirmed by ¹H NMR.

1H NMR (400 MHz, DMSO-d₆) δ ppm −0.04 (s, 9H), 0.77-0.87 (m, 2H), 1.75-1.84 (m, 3H), 2.00-2.05 (m, 2H), 2.30-2.33 (m, 1H), 2.54-2.58 (m, 2H), 2.67-2.70 (m, 1H), 2.88-2.94 (m, 3H), 3.01-3.12 (m, 1H), 3.20-3.26 (m, 2H), 3.87-3.90 (m, 2H), 7.16-7.22 (m, 2H), 7.32 (d, J=7.60 Hz, 1H) 7.56 (d, J=7.60 Hz, 1H), 7.65 (br s, 1H), 8.18-8.23 (m, 2H), 8.98 (br s, 1H).

### Step 8

### Intermediate 432: tert-butyl 6-(4-(5-(2-(2,6-dioxo-1-((2-(trimethylsilyl)ethoxy)methyl)piperidin-3-yl)-1-oxoisoindolin-4-yl)pent-4-yn-1-yl)-2-fluorobenzamido)-2-((R)-1-methylpyrrolidin-2-yl)-1H-pyrrolo[3,2-c]pyridine-1-carboxylate

To a solution of 4-(5-(2-(2,6-dioxo-1-((2-(trimethylsilyl)ethoxy)methyl)piperidin-3-yl)-1-oxoisoindolin-4-yl)pent-4-yn-1-yl)-2-fluorobenzamide (Intermediate 431, 0.34 g, 0.89 mmol) in 1,4-dioxane (5 mL) was added tert-butyl (R)-6-bromo-2-(1-methylpyrrolidin-2-yl)-1H-pyrrolo[3,2-c]pyridine-1-carboxylate (Intermediate 4, 0.34 g, 0.89 mmol) followed by cesium carbonate (0.87 g, 2.68 mmol). The reaction mixture was purged with argon for 15 min, then Pd₂(dba)₃ (0.13 g, 0.13 mmol) and Xantphos (0.16 g, 0.27 mmol) were added. The reaction mixture was purged with argon for another 10 min and stirred at 100° C. for 2 h. After completion, the reaction mixture was filtered through a celite bed, the filter pad was washed with ethyl acetate (100 mL) and the filtrate was concentrated in vacuo. The crude material was purified by combi-flash chromatography, by eluting with 90% ethyl acetate in heptane, to afford tert-butyl 6-(4-(5-(2-(2,6-dioxo-1-((2-(trimethylsilyl)ethoxy)methyl)piperidin-3-yl)-1-oxoisoindolin-4-yl)pent-4-yn-1-yl)-2-fluorobenzamido)-2-((R)-1-methylpyrrolidin-2-yl)-1H-pyrrolo[3,2-c]pyridine-1-carboxylate (Intermediate 432, 0.38 g, 49%) as a yellow solid. ¹H NMR (400 MHz, DMSO-d₆) δ ppm −0.048 (s, 9H), 0.79-0.82 (m, 2H), 1.52-1.65 (m, 2H), 1.69 (s, 9H), 1.72-1.76 (m, 1H), 1.89-2.02 (m, 2H), 2.08-2.15 (m, 2H), 2.35 (s, 3H), 2.38-2.45 (m, 2H), 2.67-2.77 (m, 2H), 2.79-2.83 (m, 2H), 3.04-3.17 (m, 3H), 3.47-3.54 (m, 2H), 3.89-3.94 (m, 1H), 4.28-4.32 (m, 1H), 4.49-4.53 (m, 1H), 5.01-5.08 (m, 2H), 5.27-5.29 (m, 1H), 6.74 (s, 1H), 7.19-7.25 (m, 2H), 7.52-7.59 (m, 1H), 7.65-7.74 (m, 3H), 8.56 (s, 1H), 8.92 (s, 1H), 10.50 (s, 1H). Mass spec m/z 876.7 [M+H]⁺.

### Step 9

### Example 94: 4-(5-(2-(2,6-dioxopiperidin-3-yl)-1-oxoisoindolin-4-yl)pent-4-yn-1-yl)-2-fluoro-N-(2-((R)-1-methylpyrrolidin-2-yl)-1H-pyrrolo[3,2-c]pyridin-6-yl)benzamide

To a solution of tert-butyl 6-(4-(5-(2-(2,6-dioxo-1-((2-(trimethylsilyl)ethoxy)methyl)piperidin-3-yl)-1-oxoisoindolin-4-yl)pent-4-yn-1-yl)-2-fluorobenzamido)-2-((R)-1-methylpyrrolidin-2-yl)-1H-pyrrolo[3,2-c]pyridine-1-carboxylate (Intermediate 432, 0.38 g, 0.433 mmol) in acetonitrile (5 mL) was added methanesulfonic acid (0.42 g, 4.33 mmol) and the reaction mixture was stirred at 50° C. for 2 h. After cooling to room temperature, N,N′-dimethylethylenediamine (0.21 g, 2.16 mmol) and triethylamine (0.88 g, 8.7 mmol) were added and the reaction mixture was stirred at room temperature for 3 h. After completion, the reaction mixture was concentrated in vacuo. The obtained residue was diluted with water (50 mL) and the resulting solid precipitate was collected by filtration and dried in vacuo to obtain the crude compound. The crude material was purified by preparative HPLC (Method A) to afford 4-(5-(2-(2,6-dioxopiperidin-3-yl)-1-oxoisoindolin-4-yl)pent-4-yn-1-yl)-2-fluoro-N-(2-((R)-1-methylpyrrolidin-2-yl)-1H-pyrrolo[3,2-c]pyridin-6-yl)benzamide (Example 94, 0.054 g, 19%) as an off-white solid. ¹H NMR (400 MHz, DMSO-d₆) δ ppm 1.81-1.89 (m, 5H), 1.92-1.95 (m, 1H), 2.12-2.14 (m, 1H), 2.16 (s, 3H), 2.25-2.31 (m, 1H), 2.44-2.48 (m, 2H), 2.53-2.62 (m, 2H), 2.81-2.85 (m, 3H), 3.14-3.17 (m, 1H), 3.31-3.35 (m, 1H), 4.31-4.40 (m, 1H), 4.47-4.52 (m, 1H), 5.10-5.19 (m, 1H), 6.40 (s, 1H), 7.20-7.26 (m, 2H), 7.53 (t, J=7.60 Hz, 1H), 7.67-7.73 (m, 3H), 8.20 (s, 1H), 8.47 (s, 1H), 10.19 (s, 1H), 11.00 (s 1H), 11.37 (s, 1H). ¹⁹F NMR (400 MHz, DMSO-d₆) δ ppm −114.24. Mass spec m/z 647.2 [M+H]⁺.

### Example 95 was Prepared Following Scheme 96

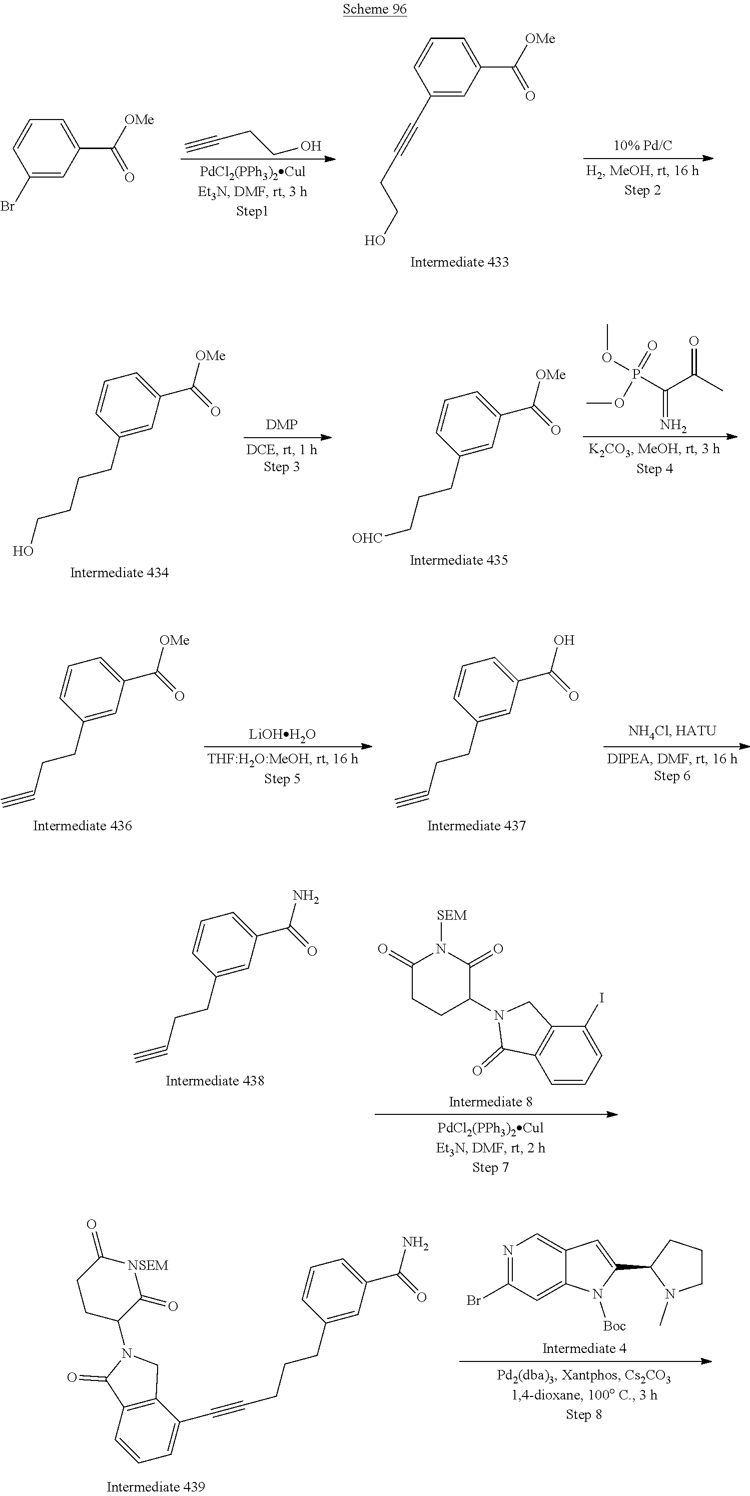

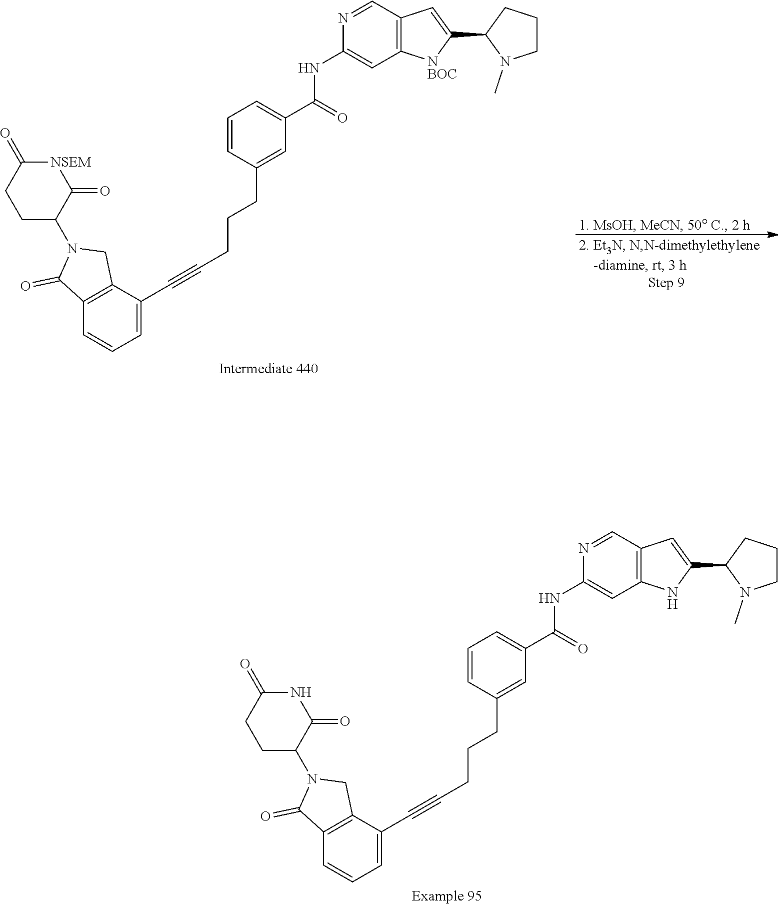

### Step 1

### Intermediate 433: methyl 3-(4-hydroxybut-1-yn-1-yl)benzoate

To a solution of methyl 3-bromobenzoate (CAS No. 618-89-3, 10.0 g, 38.2 mmol) in dimethylformamide (30 mL) was added but-3-yn-1-ol (CAS No. 927-74-2, 3.21 g, 45.8 mmol) followed by triethylamine (187.0 mL, 1340.0 mmol). The reaction mixture was purged with argon for 15 min then copper (I) iodide (0.74 g, 3.82 mmol) and PdCl₂(PPh₃)₂ (2.71 g, 3.82 mmol) were added. The reaction mixture was purged with argon for another 10 min and stirred at room temperature for 3 h. After completion, the reaction mixture was diluted water (200 mL) and extracted with ethyl acetate (3×200 mL). The combined organic layers were dried over anhydrous Na₂SO₄ and concentrated in vacuo. The crude material was purified by combi-flash chromatography, by eluting with 90% ethyl acetate in heptane, to afford methyl 3-(4-hydroxybut-1-yn-1-yl)benzoate (Intermediate 433, 6.5 g, 83%) as a yellow liquid. ¹H NMR (400 MHz, DMSO-d₆) δ ppm 2.57 (t, J=6.40 Hz, 2H), 3.60 (t, J=6.40 Hz, 2H), 3.85 (s, 3H), 7.51 (t, J=8.0 Hz, 1H), 7.65 (d, J=8.0 Hz, 1H), 7.85-7.87 (m, 2H). Exchangeable proton was not observed. Mass spec m/z 205.4 [M+H]⁺.

### Step 2

### Intermediate 434: methyl 3-(4-hydroxybutyl)benzoate

To a solution of methyl 3-(4-hydroxybut-1-yn-1-yl)benzoate (Intermediate 433, 6.5 g, 32.0 mmol) in methanol (100 mL) was added 10% Pd/C (2.5 g, 24.0 mmol) and the mixture was stirred at room temperature for 16 h under hydrogen atmosphere at 110 psi. After completion, the reaction mixture was filtered through a celite bed, the filter pad was washed with MeOH (100 mL) and the filtrate concentrated in vacuo to afford methyl 3-(4-hydroxybutyl)benzoate (Intermediate 434, 3.0 g, 45%) as a yellow liquid, which was used for the next step without further purification. ¹H NMR (400 MHz, DMSO-d₆) δ ppm 1.40-1.44 (m, 2H), 1.58-1.62 (m, 2H), 2.65 (t, J=7.60 Hz, 2H), 3.36-3.46 (m, 2H), 3.86 (s, 3H), 4.40 (t, J=5.20 Hz, 1H), 7.39-7.47 (m, 1H), 7.47-7.52 (m, 1H), 7.60-7.70 (m, 1H) 7.77-7.79 (m, 1H). Mass spec m/z 209.0 [M+H]⁺.

### Step 3

### Intermediate 435: methyl 3-(4-oxobutyl)benzoate

To a cooled (0° C.) solution of methyl 3-(4-hydroxybutyl)benzoate (Intermediate 434, 2.8 g, 13.0 mmol) in dichloromethane (30 mL) was added Dess-Martin periodinane (8.8 g, 20.0 mmol) and the reaction mixture was stirred at room temperature for 1 h. The reaction mixture was diluted saturated aqueous sodium bicarbonate solution (100 mL) and extracted with dichloromethane (3×100 mL). The combined organic layers were dried over anhydrous Na₂SO₄ filtered and concentrated in vacuo to afford methyl 3-(4-oxobutyl)benzoate (Intermediate 435, 1.8 g, 65%) as a colourless liquid, which was used for the next step without further purification. ¹H NMR (400 MHz, DMSO-d₆) δ ppm 1.80-1.91 (m, 2H), 2.45 (t, J=7.20 Hz, 2H), 2.67 (t, J=7.20 Hz, 2H), 3.85 (s, 3H), 7.41-7.52 (m, 2H), 7.76-7.80 (m, 2H), 9.66 (s, 1H).

### Step 4

### Intermediate 436: methyl 3-(pent-4-yn-1-yl)benzoate

To a solution of methyl 3-(4-oxobutyl)benzoate (Intermediate 435, 1.8 g, 8.7 mmol) in methanol (20 mL) was added potassium carbonate (2.5 g, 17.0 mmol) followed by dimethyl (1-diazo-2-oxopropyl)phosphonate (CAS No. 90965-06-3, 3.4 g, 17.0 mmol). The reaction mixture was stirred at room temperature for 3 h. After completion, the reaction mixture was concentrated in vacuo. The crude material was purified by combi-flash chromatography, by eluting with 40% ethyl acetate in heptane, to afford methyl 3-(pent-4-yn-1-yl)benzoate (Intermediate 436, 1.0 g, 57%) as a gummy oil. ¹H NMR (400 MHz, DMSO-d₆) δ ppm 1.71-1.77 (m, 2H), 2.15 (t, J=7.60 Hz, 2H), 2.73 (t, J=7.60 Hz, 2H), 2.83 (s, 1H), 3.84 (s, 3H), 7.42-7.54 (m, 2H), 7.78-7.82 (m, 2H). Mass spec m/z 202.5 [M+H]⁺.

### Step 5

### Intermediate 437: 3-(pent-4-yn-1-yl)benzoic acid

To a solution of methyl 3-(pent-4-yn-1-yl)benzoate (Intermediate 436, 1.0 g, 4.9 mmol) in tetrahydrofuran (5.0 mL), water (2.0 mL) and methanol (2.0 mL) was added lithium hydroxide hydrate (0.48 g, 20.0 mmol) and the reaction mixture was stirred at room temperature for 16 h. After completion, the reaction mixture was concentrated in vacuo. The obtained residue was diluted with water (10 mL) and acidified to pH~4 by the addition of 0.5 N aqueous HCl solution (50 mL). The resulting milky solution was filtered and the resultant solid dried in vacuo to afford 3-(pent-4-yn-1-yl)benzoic acid (Intermediate 437, 0.80 g, 80%) as an off-white solid which was used for the next step without further purification. ¹H NMR (400 MHz, DMSO-d₆) δ ppm 1.72-1.79 (m, 2H), 2.15 (t, J=7.60 Hz, 2H), 2.73 (t, J=7.60 Hz, 2H), 2.83 (s, 1H), 7.39-7.47 (m, 2H), 7.75-7.78 (m, 2H), 12.92 (br s, 1H). Mass spec m/z 187.4 [M−H]⁻.

### Step 6

### Intermediate 438: 3-(pent-4-yn-1-yl)benzamide

To a cooled (0° C.) solution of 3-(pent-4-yn-1-yl)benzoic acid (Intermediate 437, 0.80 g, 4.25 mmol) in dimethylformamide (5 mL) was added HATU (2.42 g, 6.37 mmol) and N,N-diisopropylethylamine (4 mL, 21.52 mmol). Ammonium chloride (0.90 g, 17.00 mmol) was then added and the reaction mixture stirred at room temperature for 16 h. After completion, the reaction mixture was poured in ice cold water (50 mL), the resultant precipitate was collected by filtration and dried in vacuo to afford 3-(pent-4-yn-1-yl)benzamide (Intermediate 438, 0.70 g, 88%) as an off-white solid. ¹H NMR (400 MHz, DMSO-d₆) δ ppm 1.72-1.80 (m, 2H), 2.10-2.20 (m, 2H), 2.70 (t, J=7.60 Hz, 2H), 2.81 (s, 1H), 7.28-7.35 (m, 3H), 7.62-7.72 (m, 2H), 7.95 (s, 1H).

### Step 7

### Intermediate 439: 3-(5-(2-(2,6-dioxo-1-((2-(trimethylsilyl)ethoxy)methyl)piperidin-3-yl)-1-oxoisoindolin-4-yl)pent-4-yn-1-yl)benzamide

To a solution of 3-(pent-4-yn-1-yl)benzamide (Intermediate 438, 0.30 g, 1.60 mmol) in dimethylformamide (5 mL) was added 3-(4-iodo-1-oxoisoindolin-2-yl)-1-((2-(trimethylsilyl) ethoxy)methyl)piperidine-2,6-dione (Intermediate 8, 0.80 g, 1.60 mmol) followed by triethylamine (7.86 mL, 56.07 mmol). The reaction mixture was purged with argon for 15 min then copper (I) iodide (0.03 g, 0.16 mmol) and PdCl₂(PPh₃)₂ (0.11 g, 0.16 mmol) were added. The reaction mixture was purged with argon for another 10 min and stirred at room temperature for 2 h. After completion, the reaction mixture was diluted with water (50 mL) and extracted with ethyl acetate (3×50 mL). The combined organic layers were dried over anhydrous Na₂SO₄ and concentrated in vacuo. The crude material was purified by combi-flash chromatography, by eluting with 90% ethyl acetate in heptane, to afford 3-(5-(2-(2,6-dioxo-1-((2-(trimethylsilyl)ethoxy)methyl)piperidin-3-yl)-1-oxoisoindolin-4-yl)pent-4-yn-1-yl)benzamide (Intermediate 439, 0.45 g, 50%) as a white solid. ¹H NMR (400 MHz, DMSO-d₆) δ ppm −0.04 (s, 9H), 0.79-0.85 (m, 2H), 1.87-1.94 (m, 2H), 2.03-2.10 (m, 1H), 2.41-2.42 (m, 1H), 2.66-2.75 (m, 2H), 2.76-2.83 (m, 3H), 3.02-3.11 (m, 1H), 3.48-3.52 (m, 2H), 4.27-4.32 (m, 1H), 4.48-4.52 (m, 1H), 5.01-5.08 (m, 2H), 5.26-5.30 (m, 1H), 7.31-7.42 (m, 3H), 7.54 (t, J=7.60 Hz, 1H), 7.67-7.78 (m, 4H), 7.95 (br s, 1H). Mass spec m/z 560.0 [M+H]⁺.

### Step 8

### Intermediate 440: tert-butyl 6-(3-(5-(2-(2,6-dioxo-1-((2-(trimethylsilyl)ethoxy)methyl)piperidin-3-yl)-1-oxoisoindolin-4-yl)pent-4-yn-1-yl)benzamido)-2-((R)-1-methylpyrrolidin-2-yl)-1H-pyrrolo[3,2-c]pyridine-1-carboxylate (11)

To a solution of 3-(5-(2-(2,6-dioxo-1-((2-(trimethylsilyl)ethoxy)methyl)piperidin-3-yl)-1-oxoisoindolin-4-yl)pent-4-yn-1-yl)benzamide (Intermediate 439, 0.45 g, 0.80 mmol) in 1,4-dioxane (5 mL) was added tert-butyl (R)-6-bromo-2-(1-methylpyrrolidin-2-yl)-1H-pyrrolo[3,2-c]pyridine-1-carboxylate (Intermediate 4, 0.27 g, 0.72 mmol) followed by cesium carbonate (0.46 g, 2.41 mmol). The reaction mixture was purged with argon for 15 min then Pd₂(dba)₃ (0.07 g, 0.08 mmol) and Xantphos (0.04 g, 0.08 mmol) were added. The reaction mixture was further purged with argon for 10 min and stirred at 100° C. for 3 h. After completion, the reaction mixture was concentrated in vacuo and the crude material was purified by combi-flash chromatography, by eluting with 10% MeOH in DCM, to afford tert-butyl 6-(3-(5-(2-(2,6-dioxo-1-((2-(trimethylsilyl)ethoxy)methyl)piperidin-3-yl)-1-oxoisoindolin-4-yl)pent-4-yn-1-yl)benzamido)-2-((R)-1-methylpyrrolidin-2-yl)-1H-pyrrolo[3,2-c]pyridine-1-carboxylate (Intermediate 440, 0.4 g, 58%) as a yellow solid. ¹H NMR (400 MHz, DMSO-d₆) δ ppm −0.06 (s, 9H), 0.72-0.88 (m, 2H), 1.57-1.64 (m, 1H), 1.69 (s, 9H), 1.71-1.79 (m, 2H), 1.90-2.09 (m, 3H), 2.35 (s, 3H), 2.41-2.50 (m, 2H), 2.64-2.77 (m, 3H), 2.79-2.91 (m, 3H), 3.10-3.14 (m, 1H), 3.41-3.56 (m, 3H), 3.87-3.95 (m, 1H), 4.27-4.32 (m, 1H), 4.49-4.53 (m, 1H), 4.95-5.08 (m, 2H), 5.23-5.28 (m, 1H), 6.75 (s, 1H), 7.28-7.48 (m, 2H), 7.50-7.59 (m, 1H), 7.67-7.73 (m, 2H), 7.86 (d, J=6.0 Hz, 1H), 7.93-7.96 (m, 1H), 8.58 (s, 1H), 8.92 (s, 1H), 10.70 (br s, 1H). Mass spec m/z 858.8 [M+H]⁺.

### Step 9

### Example 95: 3-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide

To a solution of tert-butyl 6-(3-(5-(2-(2,6-dioxo-1-((2-(trimethylsilyl)ethoxy)methyl)piperidin-3-yl)-1-oxoisoindolin-4-yl)pent-4-yn-1-yl)benzamido)-2-((R)-1-methylpyrrolidin-2-yl)-1H-pyrrolo[3,2-c]pyridine-1-carboxylate (Intermediate 440, 0.40 g, 0.46 mmol) in acetonitrile (5 mL) was added methanesulfonic acid (0.46 mL, 6.98 mmol) and the reaction mixture was heated at 50° C. for 2 h. After cooling to room temperature, N,N′-dimethylethylenediamine (0.44 mL, 3.72 mmol) followed by triethylamine (1.30 mL, 9.31 mmol) were added and the reaction mixture was stirred at room temperature for 3 h. The reaction mixture was then poured into ice cold water (80 mL), the resultant precipitate was collected by filtration and dried in vacuo. The crude material was purified by preparative HPLC (Method A) to afford 3-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide (Example 95, 0.06 g, 22%) as an off-white solid. ¹H NMR (400 MHz, DMSO-d₆) δ ppm 1.78-1.99 (m, 6H), 2.08-2.12 (m, 1H), 2.16 (s, 3H), 2.24-2.29 (m, 1H), 2.40-2.46 (m, 1H), 2.52-2.58 (m, 3H), 2.80-2.92 (m, 3H), 3.10-3.18 (m, 1H), 3.27-3.31 (m, 1H), 4.31-4.39 (m, 1H), 4.46-4.54 (m, 1H), 5.10-5.15 (m, 1H), 6.39 (s, 1H), 7.39-7.46 (m, 2H), 7.50-7.56 (m, 1H), 7.66-7.72 (m, 2H), 7.87 (d, J=6.40 Hz, 1H), 7.94-7.96 (m, 1H), 8.19 (s, 1H), 8.49 (s, 1H), 10.46 (br s, 1H), 10.98 (br s, 1H), 11.36 (br s, 1H). Mass spec m/z 629.0 [M+H]⁺.

### Example 96 was Prepared Following Scheme 97

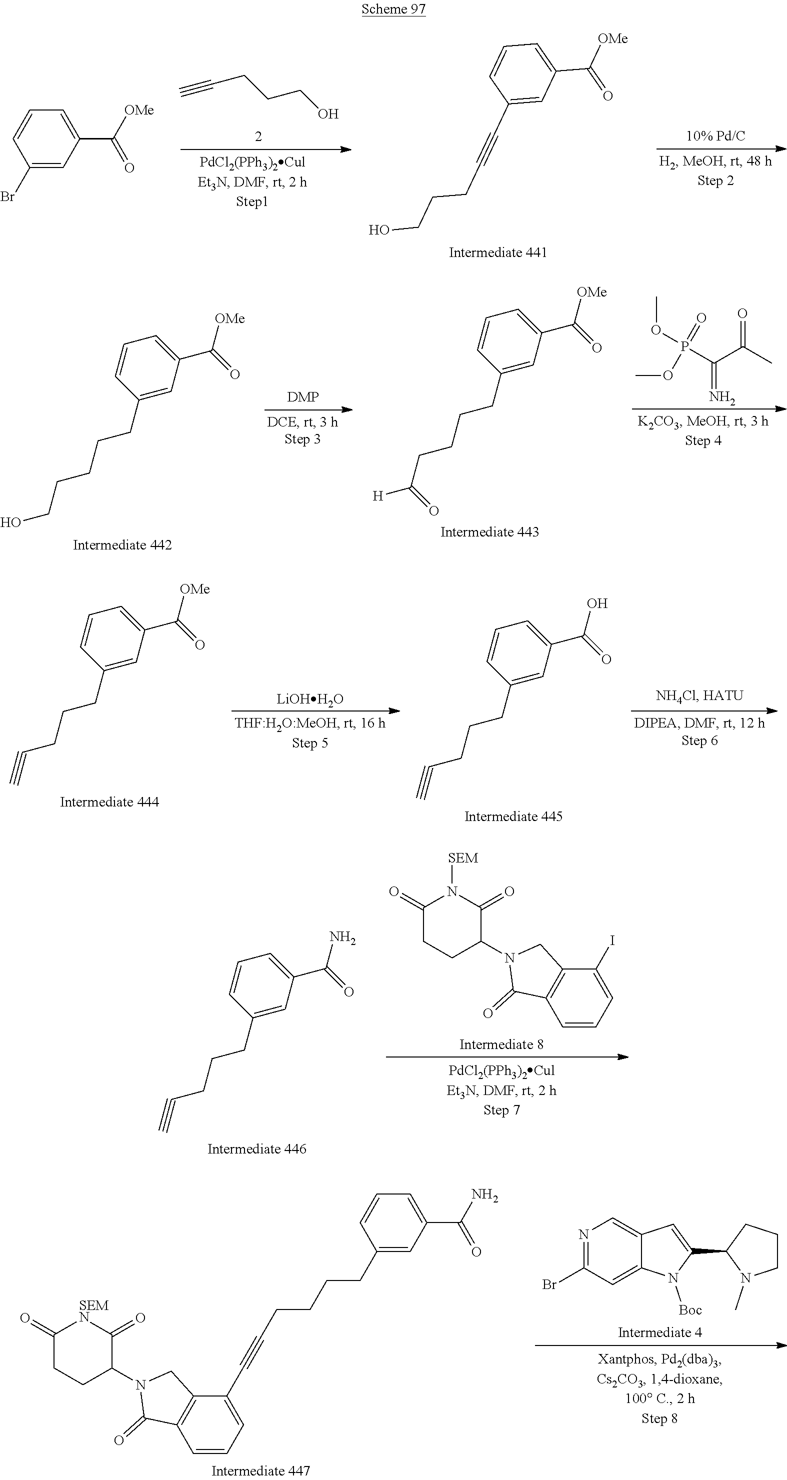

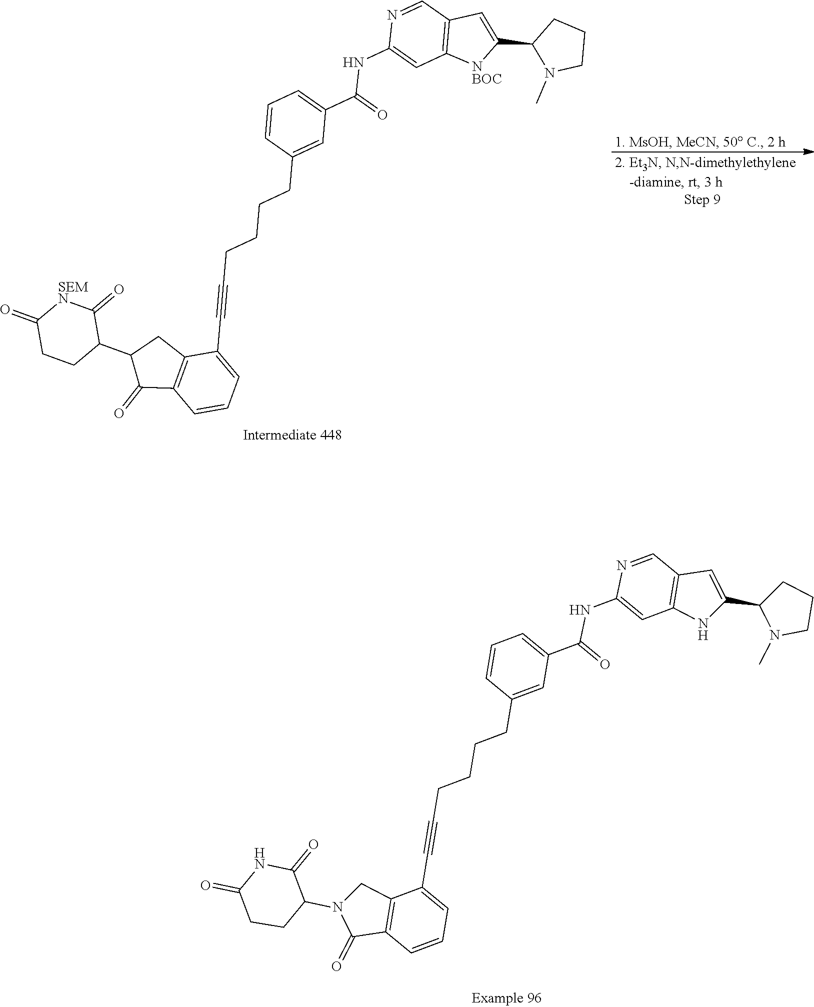

### Step 1

### Intermediate 441: methyl 3-(5-hydroxypent-1-yn-1-yl)benzoate

To a solution of methyl 3-bromobenzoate (CAS No. 618-89-3, 10.00 g, 46.51 mmol) in dimethylformamide (50 mL) was added pent-4-yn-1-ol (CAS No. 5390-04-5, 3.20 g, 46.51 mmol) followed by triethylamine (232 mL, 1674 mmol). The reaction mixture was purged with argon for 15 min then copper (I) iodide (0.8 g, 4.6 mmol) and PdCl₂(PPh₃)₂ (3.20 g, 4.6 mmol) were added. The reaction mixture was purged with argon for another 10 min and stirred at room temperature for 2 h. After completion, the reaction mixture was diluted with water (200 mL) and extracted with ethyl acetate (3×150 mL). The combined organic layers were, dried over anhydrous Na₂SO₄, and concentrated in vacuo. The crude material was purified by combi-flash column chromatography, by eluting with 2% MeOH in DCM, to afford methyl 3-(5-hydroxypent-1-yn-1-yl)benzoate (Intermediate 441, 7.0 g, 66%) as a yellow solid. The crude material was purified by combi-flash chromatography, by eluting with 2% MeOH in DCM, to afford methyl 3-(5-hydroxypent-1-yn-1-yl)benzoate (Intermediate 441, 7.0 g, 66%) as a yellow solid. ¹H NMR (400 MHz, DMSO-d₆) δ ppm 1.67-1.70 (m, 2H), 2.45-2.50 (m, 2H), 3.50-3.56 (m, 2H), 3.85 (s, 3H), 4.55 (t, J=5.20 Hz, 1H), 7.51 (d, J=8.0 Hz, 1H), 7.64 (d, J=8.0 Hz, 1H), 7.89-7.91 (m, 2H).

### Step 2

### Intermediate 442: methyl 3-(5-hydroxypentyl)benzoate

To a solution of methyl 3-(5-hydroxypent-1-yn-1-yl)benzoate (Intermediate 441, 9.0 g, 41.25 mmol) in MeOH (100 mL) was added 10% Pd/C (3.5 g) at room temperature and the reaction mixture was stirred for 48 h under 100 psi H₂ atmosphere. After completion of the reaction, the reaction mixture was filtered through a celite bed and the filtrate was concentrated in vacuo to afford methyl 3-(5-hydroxypentyl)benzoate (Intermediate 442, 6.70 g, 73%) as a light yellow solid. ¹H NMR (400 MHz, DMSO-d₆) δ ppm 1.27-1.31 (m, 2H), 1.41-1.48 (m, 2H), 1.55-1.60 (m, 2H), 2.64 (t, J=7.60 Hz, 2H), 3.34-3.39 (m, 2H), 3.84 (s, 3H), 4.33 (t, J=5.60 Hz, 1H), 7.42-7.51 (m, 2H), 7.76-7.79 (m, 2H).

### Step 3

### Intermediate 443: methyl 3-(5-oxopentyl)benzoate

To a cooled (0° C.) solution of methyl 3-(5-hydroxypentyl)benzoate (Intermediate 442, 6.70 g, 30 mmol) in dichloromethane (100 mL) was added Dess-Martin periodinane (20.0 g, 45 mmol) and the reaction mixture was stirred at room temperature for 3 h. After completion, the reaction mixture was diluted with saturated aqueous sodium bicarbonate solution (100 mL) and saturated aqueous sodium thiosulphate solution (100 mL) and extracted with dichloromethane (2×200 mL). The combined organic layers were dried over Na₂SO₄, filtered, and concentrated in vacuo to afford methyl 3-(5-oxopentyl)benzoate (Intermediate 443, 5.5 g, 83%) as a yellow solid which was used into next step without purification. ¹H NMR (400 MHz, DMSO-d₆) δ ppm 1.50-1.60 (m, 4H), 2.44-2.50 (m, 2H), 2.65 (t, J=7.24 Hz, 2H), 3.84 (s, 3H), 7.41-7.49 (m, 2H), 7.74-7.78 (m, 2H), 9.65 (s, 1H).

### Step 4

### Intermediate 444: methyl 3-(hex-5-yn-1-yl)benzoate

To a solution of methyl 3-(5-oxopentyl)benzoate (Intermediate 443, 10.0 g, 45.39 mmol) in methanol (50 mL) was added potassium carbonate (12.53 g, 90.79 mmol) followed by dimethyl (1-diazo-2-oxopropyl)phosphonate (CAS No. 90965-06-3, 13.08 g, 68.09 mmol). The reaction mixture was stirred at room temperature for 3 h. After completion, the reaction mixture was diluted with water (100 mL) and extracted with diethyl ether (2×100 mL). The combined organic layers were dried over anhydrous Na₂SO₄, filtered, and concentrated in vacuo. The crude material was purified by combi-flash chromatography, by eluting with 5-10% ethyl acetate in heptane, to afford methyl 3-(hex-5-yn-1-yl)benzoate (Intermediate 444, 9.0 g, 92%) as a yellow oil. ¹H NMR (400 MHz, DMSO-d₆) δ ppm 1.43-1.48 (m, 2H), 1.62-169 (m, 2H), 2.16-2.20 (m, 2H), 2.65 (t, J=8.0 Hz, 2H), 2.74 (t, J=2.80 Hz, 1H), 3.84 (s, 3H), 7.41-7.49 (m, 2H), 7.77-7.79 (m, 2H).

### Step 5

### Intermediate 445: 3-(hex-5-yn-1-yl)benzoic acid

To a solution of methyl 3-(hex-5-yn-1-yl)benzoate (Intermediate 444, 9.0 g, 41.61 mmol) in methanol (40 mL), tetrahydrofuran (40 mL) and water (20 mL) was added lithium hydroxide hydrate (1.30 g, 166.44 mmol) and the reaction mixture was stirred at room temperature for 16 h. The reaction mixture was then concentrated in vacuo. The obtained residue was diluted with water (50 mL) and then acidified to pH~2 by the addition of 0.5 N aqueous HCl solution. The resultant precipitate was collected by filtration and dried in vacuo to afford 3-(hex-5-yn-1-yl)benzoic acid (Intermediate 445, 7.0 g, 83%) as an off-white solid, which was used for the next step without further purification. ¹H NMR (400 MHz, DMSO-d₆) δ ppm 1.42-1.49 (m, 2H), 1.62-1.70 (m, 2H), 2.16-2.20 (m, 2H), 2.65 (t, J=8.0 Hz, 2H), 2.75 (t, J=2.40 Hz, 1H), 7.38-7.46 (m, 2H), 7.75-7.80 (m, 2H), 12.80 (br s, 1H). Mass spec m/z 201.4 [M−H]⁻.

### Step 6

### Intermediate 446: 3-(hex-5-yn-1-yl)benzamide

To a solution of 3-(hex-5-yn-1-yl)benzoic acid (Intermediate 445, 7.0 g, 34.62 mmol) in dimethylformamide (30 mL) was added HATU (19.75 g, 51.93 mmol) and N,N-diisopropylethylamine (18.15 mL, 103.90 mmol) and the reaction mixture was stirred at room temperature for 20 min. Ammonium chloride (7.4 g, 138.50 mmol) was then added, and the reaction mixture was stirred at room temperature for 12 h. The reaction mixture was quenched with ice cold water (100 mL) and extracted with ethyl acetate (2×100 mL). The combined organic layers were dried over anhydrous Na₂SO₄, filtered, and concentrated in vacuo. The crude residue was purified by combi-flash chromatography, by eluting with 70-80% ethyl acetate in heptane, to afford 3-(hex-5-yn-1-yl)benzamide (Intermediate 446, 5.0 g, 69%) as an off-white solid. ¹H NMR (400 MHz, DMSO-d₆) δ ppm 1.44-1.49 (m, 2H), 1.63-1.69 (m, 2H), 2.16-2.20 (m, 2H), 2.63 (t, J=7.60 Hz, 2H), 2.74 (t, J=2.40 Hz, 1H), 7.31 (br s, 1H), 7.33-7.37 (m, 2H), 7.67-7.72 (m, 2H), 7.93 (br s, 1H). Mass spec m/z 200.5 [M−H]⁻.

### Step 7

### Intermediate 447: 3-(6-(2-(2,6-dioxo-1-((2-(trimethylsilyl)ethoxy)methyl)piperidin-3-yl)-1-oxoisoindolin-4-yl)hex-5-yn-1-yl)benzamide

To a solution of 3-(hex-5-yn-1-yl)benzamide (Intermediate 446, 0.4 g, 1.99 mmol) in dimethylformamide (10 mL) was added 3-(4-iodo-1-oxoisoindolin-2-yl)-1-((2-(trimethylsilyl)ethoxy)methyl)piperidine-2,6-dione (Intermediate 8, 1.0 g, 1.99 mmol) followed by triethylamine (7.32 g, 71.94 mmol). The reaction mixture was purged with argon for 15 min then copper (I) iodide (0.036 g, 0.19 mmol) and PdCl₂(PPh₃)₂ (0.133 g, 0.19 mmol) were added. The reaction mixture was purged with argon for another 10 min and stirred at room temperature for 3 h. After completion, the reaction mixture was diluted with water (100 mL) and extracted with ethyl acetate (3×90 mL). The combined organic layers were dried over anhydrous Na₂SO₄, and concentrated in vacuo. The crude material was purified by combi-flash chromatography, by eluting with 60% ethyl acetate in heptane, to afford 3-(6-(2-(2,6-dioxo-1-((2-(trimethylsilyl)ethoxy)methyl)piperidin-3-yl)-1-oxoisoindolin-4-yl)hex-5-yn-1-yl)benzamide (Intermediate 447, 1.0 g, 87%) as a yellow solid. ¹H NMR (400 MHz, DMSO-d₆) δ ppm −0.04 (s, 9H), 0.77-0.87 (m, 2H), 1.55-1.63 (m, 2H), 1.76 (m, 2H), 1.99-2.08 (m, 1H), 2.29-2.41 (m, 2H), 2.63-2.74 (m, 4H), 3.01-3.12 (m, 1H), 3.48-3.57 (m, 2H), 4.22-4.27 (m, 1H), 4.42-4.47 (m, 1H), 5.01.5.08 (m, 2H), 5.24-5.28 (m, 1H), 7.31 (br s, 1H), 7.33-7.39 (m, 2H), 7.50 (d, J=7.60 Hz, 1H), 7.63-7.75 (m, 4H), 7.94 (d, J=10 Hz, 1H). Mass spec m/z 572.2 [M−H]⁻. Step 8

### Intermediate 448: tert-butyl 6-(3-(6-(2-(2,6-dioxo-1-((2-(trimethylsilyl)ethoxy)methyl)piperidin-3-yl)-1-oxoisoindolin-4-yl)hex-5-yn-1-yl)benzamido)-2-((R)-1-methylpyrrolidin-2-yl)-1H-pyrrolo[3,2-c]pyridine-1-carboxylate

To a solution of 3-(6-(2-(2,6-dioxo-1-((2-(trimethylsilyl)ethoxy)methyl)piperidin-3-yl)-1-oxoisoindolin-4-yl)hex-5-yn-1-yl)benzamide (Intermediate 447, 0.8 g, 1.05 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.50 g, 1.32 mmol) followed by cesium carbonate (1.28 g, 3.94 mmol). The reaction mixture was purged with argon for 15 min then Pd₂(dba)₃ (0.137 g, 0.15 mmol) and Xantphos (0.174 g, 0.3 mmol) were added. The reaction mixture was purged with argon for another 10 min and stirred at 100° C. for 2 h. After completion, the reaction mixture was filtered through a celite bed, the filter pad was washed with dichloromethane (30 mL) and the filtrate was concentrated in vacuo. The crude material was purified by combi-flash column chromatography, by eluting with 90% ethyl acetate in heptane, to afford tert-butyl 6-(3-(6-(2-(2,6-dioxo-1-((2-(trimethylsilyl)ethoxy)methyl)piperidin-3-yl)-1-oxoisoindolin-4-yl)hex-5-yn-1-yl)benzamido)-2-((R)-1-methylpyrrolidin-2-yl)-1H-pyrrolo[3,2-c]pyridine-1-carboxylate (Intermediate 448, 0.78 g, 68%) as a yellow solid. ¹H NMR (400 MHz, DMSO-d₆) δ ppm −0.081 (s, 9H), 0.75-0.80 (m, 2H), 1.58-1.62 (m, 3H), 1.70 (s, 9H), 1.71-1.79 (m, 5H), 1.89-2.02 (m, 1H), 2.24-2.28 (m, 2H), 2.32 (m, 3H), 2.47-2.53 (m, 2H), 2.68-2.74 (m, 3H), 2.91-3.12 (m, 2H), 3.45-3.49 (m, 2H), 3.82-3.87 (m, 1H), 4.19-4.24 (m, 1H), 4.39-4.43 (m, 1H), 4.95-5.01 (m, 2H), 5.20-5.24 (m, 1H), 6.72 (s, 1H), 7.34-7.38 (m, 2H), 7.48 (t, J=7.60 Hz, 1H), 7.61-7.68 (m, 2H), 7.80 (d, J=6.80 Hz, 1H), 7.81-7.89 (m, 1H), 8.55 (s, 1H), 8.89 (s, 1H), 10.63 (s, 1H). Mass spec m/z 873.2 [M+H]⁺.

### Step 9

### Example 96: 3-(6-(2-(2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide

To a solution of tert-butyl 6-(3-(6-(2-(2,6-dioxo-1-((2-(trimethylsilyl)ethoxy)methyl)piperidin-3-yl)-1-oxoisoindolin-4-yl)hex-5-yn-1-yl)benzamido)-2-((R)-1-methylpyrrolidin-2-yl)-1H-pyrrolo[3,2-c]pyridine-1-carboxylate (Intermediate 448, 0.78 g, 0.89 mmol) in acetonitrile (10 mL) was added methanesulfonic acid (0.88 g, 8.93 mmol) and the reaction mixture was stirred at 50° C. for 2 h. After cooling to room temperature, N,N′-dimethylethylenediamine (0.44 g, 4.447 mmol) and triethylamine (1.82 g, 17.87 mmol) were added and the reaction mixture was stirred at room temperature for 3 h. After completion, the reaction mixture was concentrated in vacuo. The obtained residue was diluted with water (50 mL), the resultant precipitate was collected by filtration and dried in vacuo. The crude material was purified by preparative HPLC (Method A) to afford 3-(6-(2-(2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide (Example 96, 0.155 g, 27%) as an off-white solid. ¹H NMR (400 MHz, DMSO-d₆) δ ppm 1.62-1.65 (m, 2H), 1.80-1.84 (m, 5H), 1.92-2.09 (m, 1H), 2.17 (s, 3H), 2.26-2.33 (m, 1H), 2.45-2.51 (m, 1H), 2.52-2.57 (m, 4H), 2.70-2.73 (m, 2H), 2.75-2.78 (m, 1H), 3.14-3.32 (m, 1H), 3.33-35 (m, 1H), 4.27-4.32 (m, 1H), 4.42-4.47 (m, 1H), 5.10-5.19 (m, 1H), 6.40 (s, 1H), 7.40-7.42 (m, 2H), 7.51 (t, J=8.0 Hz, 1H), 7.64-7.66 (m, 1H), 7.70 (d, J=7.60 Hz, 1H), 7.85-7.88 (m, 1H), 7.90-7.94 (m, 1H), 8.20 (s, 1H), 8.50 (s, 1H), 10.42 (s, 1H), 11.10 (s 1H), 11.41 (s, 1H). Mass spec m/z 643.0 [M+H]⁺.

### Example 97 was Prepared Following Scheme 98

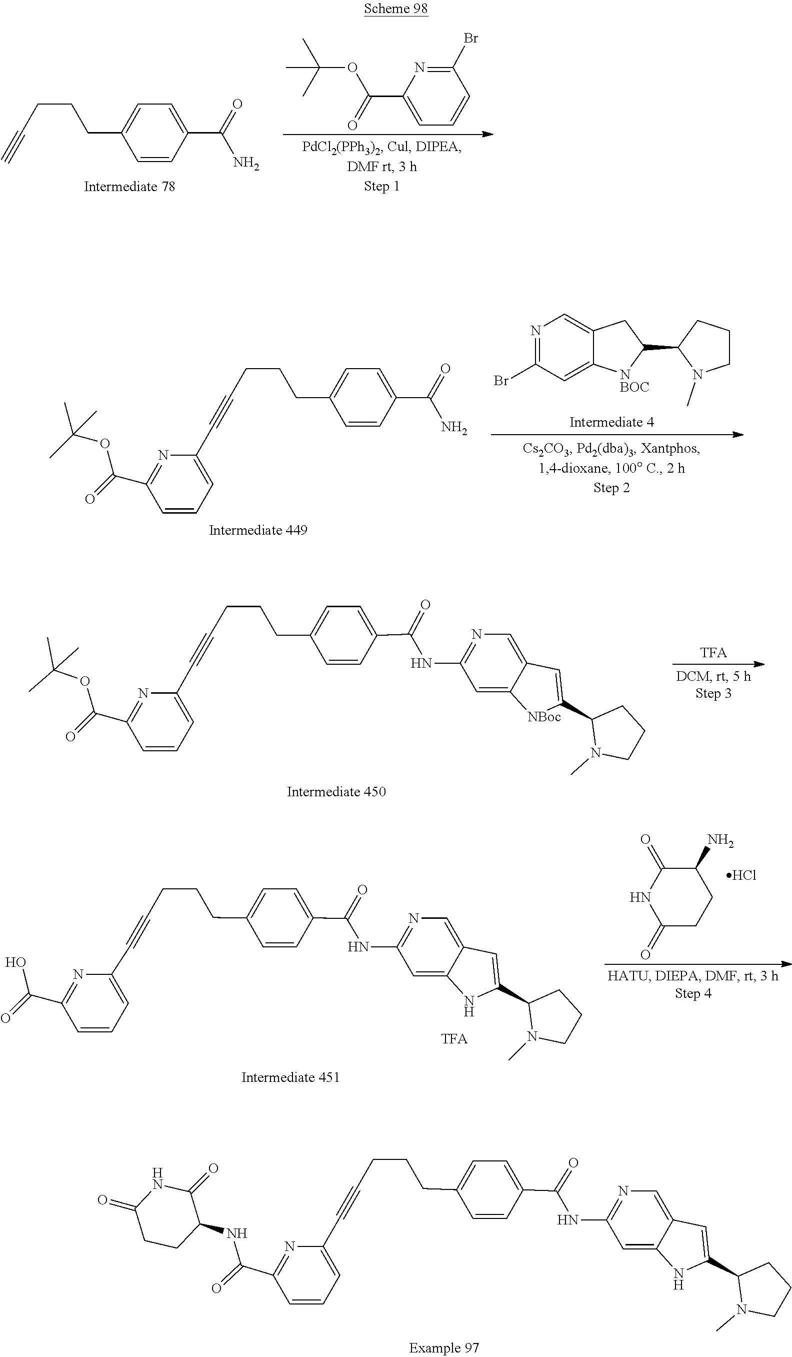

### Step 1

### Intermediate 449: tert-butyl 6-(5-(4-carbamoylphenyl)pent-1-yn-1-yl)picolinate

To a solution of 4-(pent-4-yn-1-yl)benzamide (Intermediate 78, 4.0 g, 21.36 mmol) and tert-butyl 6-bromopicolinate (CAS No. 910044-07-4, 5.51 g, 21.36 mmol) in dimethylformamide (40 mL) was added triethylamine (108 mL, 769.7 mmol). The reaction mixture was purged with argon for 15 min then copper (I) iodide (0.41 g, 2.13 mmol) and PdCl₂(PPh₃)₂ (1.51 g, 2.13 mmol) were added. The reaction mixture was purged with argon for another 10 min and stirred at room temperature for 3 h. The reaction mixture was diluted with water (200 mL) and extracted with ethyl acetate (3×100 mL). The combined organic layers were dried over anhydrous Na₂SO₄ and concentrated in vacuo. The crude material was purified by combi-flash chromatography, by eluting with 30% ethyl acetate in heptane, to afford tert-butyl 6-(5-(4-carbamoylphenyl)pent-1-yn-1-yl)picolinate (Intermediate 449, 3.6 g, 30%) as a yellow solid. ¹H NMR (400 MHz, DMSO-d₆) δ ppm 1.55 (s, 9H), 1.60-1.63 (m, 2H), 1.86-1.92 (m, 2H), 2.77-2.80 (m, 2H), 7.22 (br s, 1H), 7.30 (d, J=8.0 Hz, 2H), 7.44 (br s, 1H), 7.65-7.70 (m, 1H), 7.81 (d, J=8.0 Hz, 2H), 7.90-7.97 (m, 2H). Mass spec: m/z 365.4 [M+H]⁺.

### Step 2

### Intermediate 450: tert-butyl (R)-6-(4-(5-(6-(tert-butoxycarbonyl)pyridin-2-yl)pent-4-yn-1-yl)benzamido)-2-(1-methylpyrrolidin-2-yl)-1H-pyrrolo[3,2-c]pyridine-1-carboxylate

To a solution of tert-butyl 6-(5-(4-carbamoylphenyl)pent-1-yn-1-yl)picolinate (Intermediate 449, 0.76 g, 2.10 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 1.0 g, 2.63 mmol) followed by cesium carbonate (2.57 g, 7.88 mmol. The reaction mixture was purged with argon for 15 min then Pd₂(dba)₃ (0.37 g, 0.39 mmol) and Xantphos (0.45 g, 0.78 mmol) were added. The reaction mixture was purged with argon for another 10 min and stirred at 100° C. for 2 h. The reaction mixture was filtered through a celite bed and the filter pad was washed with ethyl acetate (200 mL). The filtrate was concentrated in vacuo and the crude material purified by combi-flash chromatography, by eluting with 90% ethyl acetate in heptane, to afford tert-butyl (R)-6-(4-(5-(6-(tert-butoxycarbonyl)pyridin-2-yl)pent-4-yn-1-yl)benzamido)-2-(1-methylpyrrolidin-2-yl)-1H-pyrrolo[3,2-c]pyridine-1-carboxylate (Intermediate 450, 1.1 g, 63%) as a yellow solid. ¹H NMR (400 MHz, DMSO-d₆) δ ppm 1.53 (s, 9H), 1.61-1.64 (m, 2H), 1.66 (s, 9H), 1.69-1.73 (m, 2H), 1.88-1.93 (m, 2H), 2.32 (s, 3H), 2.33-2.40 (m, 2H), 2.47-2.49 (m, 1H), 2.77-2.86 (m, 2H), 3.09-3.15 (m, 1H), 3.86-3.91 (m, 1H), 6.71 (s, 1H), 7.26-7.34 (m, 1H), 7.35 (d, J=8.0 Hz, 1H), 7.56-7.65 (m, 3H), 7.86-7.91 (m, 1H), 7.97 (d, J=8.0 Hz, 1H), 8.55 (s, 1H), 8.89 (s, 1H), 10.57 (br s, 1H). Mass spec: m/z 664.4 [M+H]⁺.

### Step 3

### Intermediate 451: (R)-6-(5-(4-((2-(1-methylpyrrolidin-2-yl)-1H-pyrrolo[3,2-c]pyridin-6-yl)carbamoyl)phenyl)pent-1-yn-1-yl)picolinic acid trifluoroacetate

To a solution of tert-butyl (R)-6-(4-(5-(6-(tert-butoxycarbonyl)pyridin-2-yl)pent-4-yn-1-yl)benzamido)-2-(1-methylpyrrolidin-2-yl)-1H-pyrrolo[3,2-c]pyridine-1-carboxylate (Intermediate 450, 1.10 g, mmol) in dichloromethane (30 mL) was added trifluoroacetic acid (17 mL, 222.0 mmol) and the reaction mixture was stirred at room temperature for 5 h. The reaction mixture was concentrated in vacuo to afford (R)-6-(5-(4-((2-(1-methylpyrrolidin-2-yl)-1H-pyrrolo[3,2-c]pyridin-6-yl)carbamoyl)phenyl)pent-1-yn-1-yl)picolinic acid trifluoroacetate (Intermediate 451, 1.7 g, 85%) as a brown solid, which was used for the next step without further purification. Mass spec: m/z 508.2 [M+H]⁺.

### Step 4

### Example 97: N—((S)-2,6-dioxopiperidin-3-yl)-6-(5-(4-((2-((R)-1-methylpyrrolidin-2-yl)-1H-pyrrolo[3,2-c]pyridin-6-yl)carbamoyl)phenyl)pent-1-yn-1-yl)picolinamide

To a cooled (0° C.) solution of (R)-6-(5-(4-((2-(1-methylpyrrolidin-2-yl)-1H-pyrrolo[3,2-c]pyridin-6-yl)carbamoyl)phenyl)pent-1-yn-1-yl)picolinic acid trifluoroacetate (Intermediate 451, 3.1 g, 5.1 mmol) in dimethylformamide (30 mL) was added HATU (2.9 g, 7.7 mmol) and N,N-diisopropylethylamine (5.0 mL, 26.0 mmol) and the reaction mixture was stirred at room temperature for 15 min. (S)-3-aminopiperidine-2,6-dione hydrochloride (CAS No. 25181-50-4, 1.3 g, 6.1 mmol) was then added. The reaction mixture stirred at room temperature for 3 h, then poured into ice cold water (50 mL). The resultant precipitate was collected by filtration and dried in vacuo. The crude material was purified by preparative HPLC (Method A) to afford N—((S)-2,6-dioxopiperidin-3-yl)-6-(5-(4-((2-((R)-1-methylpyrrolidin-2-yl)-1H-pyrrolo[3,2-c]pyridin-6-yl)carbamoyl)phenyl)pent-1-yn-1-yl)picolinamide (Example 97, 0.95 g, 30%) as an off-white solid. ¹H NMR (400 MHz, DMSO-d₆) δ ppm 1.74-2.05 (m, 7H), 2.13-2.15 (m, 1H), 2.16 (s, 3H), 2.22-2.29 (m, 2H), 2.51-2.54 (m, 2H), 2.78-2.88 (m, 3H), 3.12-3.14 (m, 1H), 3.25-3.31 (m, 1H), 4.77-4.84 (m, 1H), 6.39 (s, 1H), 7.39 (d, J=8.0 Hz, 2H), 7.70-7.73 (m, 1H), 7.95-8.05 (m, 4H), 8.19 (s, 1H), 8.50 (s, 1H), 8.96 (d, J=8.0 Hz, 1H), 10.37 (br s, 1H), 10.92 (br s, 1H), 11.37 (br s, 1H). Mass spec: m/z 618.2 [M+H]⁺.

### Example 98 was Prepared Following Scheme 99

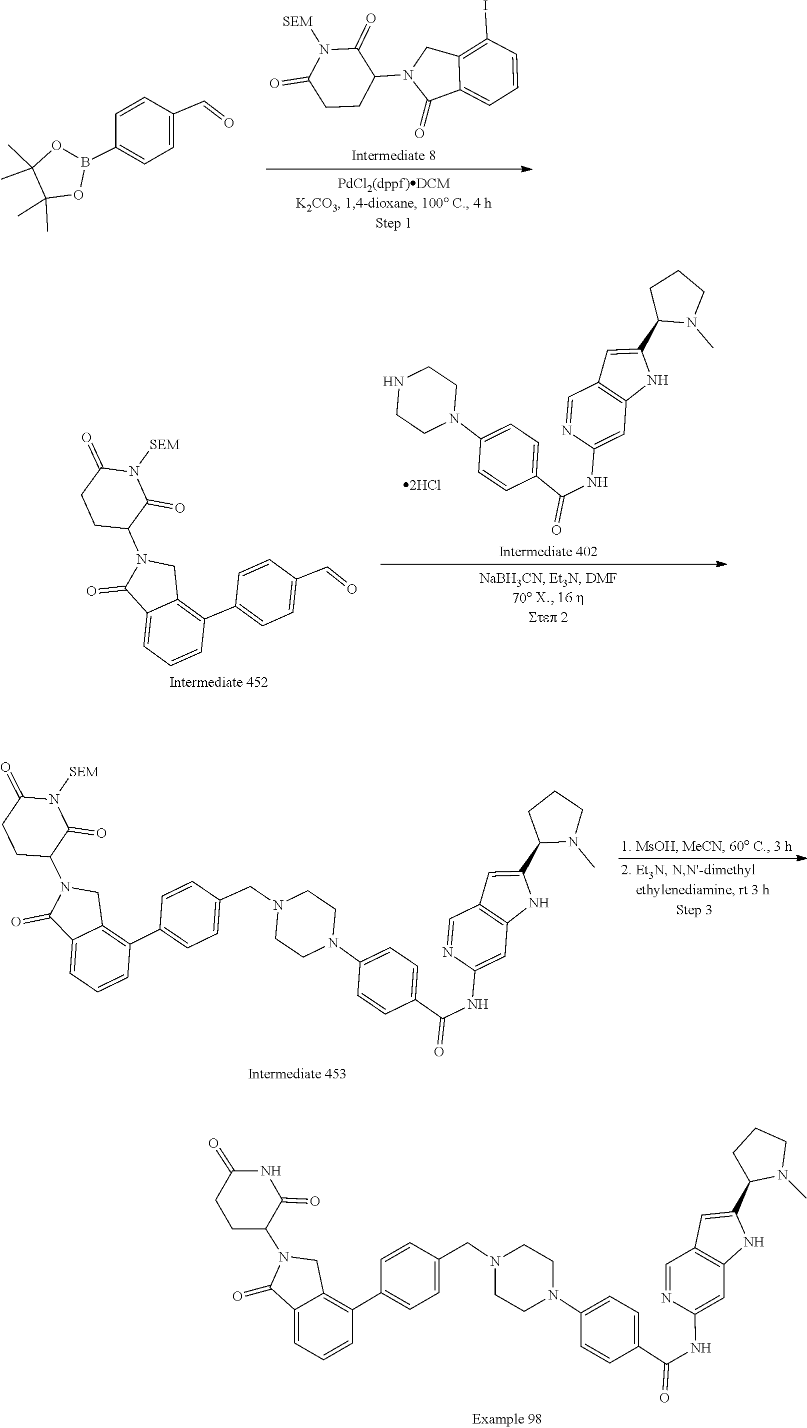 [SMILES: CC1(C)OB(c2ccc(C=O)cc2)OC1(C)C ; C[Si](C)(C)CCOCN1C(=O)CCC(N2Cc3c(I)cccc3C2=O)C1=O ; C[Si](C)(C)CCOCN1C(=O)CCC(N2Cc3c(cccc3-c3ccc(C=O)cc3)C2=O)C1=O ; CN1CCC[C@@H]1c1cc2cnc(NC(=O)c3ccc(N4CCNCC4)cc3)cc2[nH]1 ; CN1CCC[C@@H]1c1cc2cnc(NC(=O)c3ccc(N4CCN(Cc5ccc(-c6cccc7c6CN(C6CCC(=O)N(COCC[Si](C)(C)C)C6=O)C7=O)cc5)CC4)cc3)cc2[nH]1 ; CN1CCC[C@@H]1c1cc2cnc(NC(=O)c3ccc(N4CCN(Cc5ccc(-c6cccc7c6CN(C6CCC(=O)NC6=O)C7=O)cc5)CC4)cc3)cc2[nH]1]

### Step 1

### Intermediate 452: 4-(2-(2,6-dioxo-1-((2-(trimethylsilyl)ethoxy)methyl)piperidin-3-yl)-1-oxoisoindolin-4-yl)benzaldehyde

To a solution of 4-(4,4,5,5-tetramethyl-1,3,2-dioxaborolan-2-yl)benzaldehyde (CAS No. 128376-64-7, 1.1 g, 4.8 mmol) and 3-(4-iodo-1-oxoisoindolin-2-yl)-1-((2-(trimethylsilyl)ethoxy)methyl)piperidine-2,6-dione (Intermediate 8, 2.0 g, 4.0 mmol) in 1,4-dioxane (20 mL) was added potassium carbonate (1.7 g, 12.0 mmol). The reaction mixture was purged with argon for 15 min then PdCl₂(dppf) DCM (0.33 g, 0.40 mmol) was added. The reaction mixture was purged with argon for another 10 min and stirred at 100° C. for 4 h. After completion, the reaction mixture was concentrated in vacuo. The crude material was purified by combi-flash chromatography, by eluting with 40% ethyl acetate in heptane, to afford 4-(2-(2,6-dioxo-1-((2-(trimethylsilyl)ethoxy)methyl)piperidin-3-yl)-1-oxoisoindolin-4-yl)benzaldehyde (Intermediate 452, 1.6 g, 84%) as a pale-yellow semi solid. ¹H NMR (400 MHz, DMSO-d₆) δ ppm −0.08 (s, 9H), 0.77-0.82 (m, 2H), 1.98-2.05 (m, 1H), 2.39-2.49 (m, 1H), 2.75-2.80 (m, 1H), 3.02-3.12 (m, 1H), 3.48-3.51 (m, 2H), 4.31-4.37 (m, 1H), 4.70-4.75 (m, 1H), 4.98-5.06 (m, 2H), 5.27-5.32 (m, 1H), 7.68-7.79 (m, 1H), 7.77-7.82 (m, 1H), 7.79-7.85 (m, 3H), 8.01-8.04 (m, 2H), 10.09 (br s, 1H). Mass spec m/z 477.4 [M−H]⁻.

### Step 2

### Intermediate 453: 4-(4-(4-(2-(2,6-dioxo-1-((2-(trimethylsilyl)ethoxy)methyl)piperidin-3-yl)-1-oxoisoindolin-4-yl)benzyl)piperazin-1-yl)-N-(2-((R)-1-methylpyrrolidin-2-yl)-1H-pyrrolo[3,2-c]pyridin-6-yl)benzamide

To a cooled (0° C.) solution of 4-(2-(2,6-dioxo-1-((2-(trimethylsilyl)ethoxy)methyl)piperidin-3-yl)-1-oxoisoindolin-4-yl)benzaldehyde (Intermediate 452, 0.50 g, 1.04 mmol) in dimethylformamide (6 mL) was added (R)—N-(2-(1-methylpyrrolidin-2-yl)-1H-pyrrolo[3,2-c]pyridin-6-yl)-4-(piperazin-1-yl)benzamide dihydrochloride (Intermediate 402, 0.55 g, 1.25 mmol) and sodium cyanoborohydride (0.20 g, 3.13 mmol) and the reaction mixture was stirred at 70° C. for 16 h. After completion, the reaction mixture was quenched with ice cold water (50 mL), the resultant precipitate was collected by filtration and dried in vacuo to afford 4-(4-(4-(2-(2,6-dioxo-1-((2-(trimethylsilyl)ethoxy)methyl)piperidin-3-yl)-1-oxoisoindolin-4-yl)benzyl)piperazin-1-yl)-N-(2-((R)-1-methylpyrrolidin-2-yl)-1H-pyrrolo[3,2-c]pyridin-6-yl)benzamide (Intermediate 453, 0.60 g) as an off-white solid, which was used for the next step without further purification. ¹H NMR (400 MHz, DMSO-d₆) δ ppm 0.06 (s, 9H), 0.80-0.95 (m, 2H), 1.15-1.19 (m, 2H), 1.81-2.01 (m, 2H), 2.05-2.15 (m, 2H), 2.15-2.19 (m, 1H), 2.22 (s, 3H), 2.73-2.88 (m, 3H), 3.12-3.22 (m, 4H), 3.51-3.63 (m, 3H), 3.65 (s, 2H), 4.38-4.46 (m, 2H), 4.72-4.77 (m, 2H), 5.01-5.12 (m, 3H), 5.33-5.37 (m, 2H), 6.44 (s, 1H), 7.02-7.10 (m, 2H), 7.49-7.54 (m, 2H), 7.58-7.67 (m, 3H), 7.71-7.94 (m, 3H), 8.01-8.07 (m, 2H), 8.23 (s, 1H), 10.18 (br s, 1H), 11.40 (br s, 1H). Mass spec m/z 867.0 [M+H]⁺.

### Step 3

### Example 98: 4-(4-(4-(2-(2,6-dioxopiperidin-3-yl)-1-oxoisoindolin-4-yl)benzyl)piperazin-1-yl)-N-(2-((R)-1-methylpyrrolidin-2-yl)-1H-pyrrolo[3,2-c]pyridin-6-yl)benzamide

To a solution of 4-(4-(4-(2-(2,6-dioxo-1-((2-(trimethylsilyl)ethoxy)methyl)piperidin-3-yl)-1-oxoisoindolin-4-yl)benzyl)piperazin-1-yl)-N-(2-((R)-1-methylpyrrolidin-2-yl)-1H-pyrrolo[3,2-c]pyridin-6-yl)benzamide (Intermediate 453, 0.55 g, 0.63 mmol) in acetonitrile (5 mL) was added methanesulfonic acid (0.42 mL, 6.34 mmol) and the reaction mixture was stirred at 60° C. for 3 h. After cooling to room temperature, N,N′-dimethylethylenediamine (0.38 mL, 3.17 mmol) followed by triethylamine (1.78 mL, 12.69 mmol) were added, and the reaction mixture was stirred at room temperature for 3 h. After completion, the reaction mixture was concentrated in vacuo and the residue was diluted with ice cold water (50 mL). The resultant precipitate was collected by filtration and dried in vacuo. The crude material was purified by preparative HPLC (Method A) to afford 4-(4-(4-(2-(2,6-dioxopiperidin-3-yl)-1-oxoisoindolin-4-yl)benzyl)piperazin-1-yl)-N-(2-((R)-1-methylpyrrolidin-2-yl)-1H-pyrrolo[3,2-c]pyridin-6-yl)benzamide (Example 98, 0.08 g, 18%) as an off-white solid. ¹H NMR (400 MHz, DMSO-d₆) δ ppm 1.77-1.91 (m, 3H), 1.97-2.01 (m, 1H), 2.11-2.14 (m, 1H), 2.16 (s, 3H), 2.23-2.29 (m, 1H), 2.42-2.47 (m, 2H), 2.54-2.61 (m, 6H), 2.86-2.96 (m, 2H), 3.11-3.17 (m, 1H), 3.26-3.30 (m, 2H), 3.61 (s, 2H), 4.41-4.45 (m, 1H), 4.61-4.66 (m, 1H), 5.12-5.17 (m, 1H), 6.37 (s, 1H), 6.98 (d, J=8.94 Hz, 2H), 7.48 (d, J=8.25 Hz, 2H), 7.59 (d, J=8.25 Hz, 2H), 7.62-7.68 (m, 1H), 7.72-7.76 (m, 2H), 7.96 (d, J=8.94 Hz, 2H), 8.17 (s, 1H), 8.47 (s, 1H), 10.09 (br s, 1H), 10.96 (br s, 1H), 11.32 (br s, 1H). Mass spec m/z 737.0 [M+H]⁺.

### Example 99 was Prepared Following Scheme 100

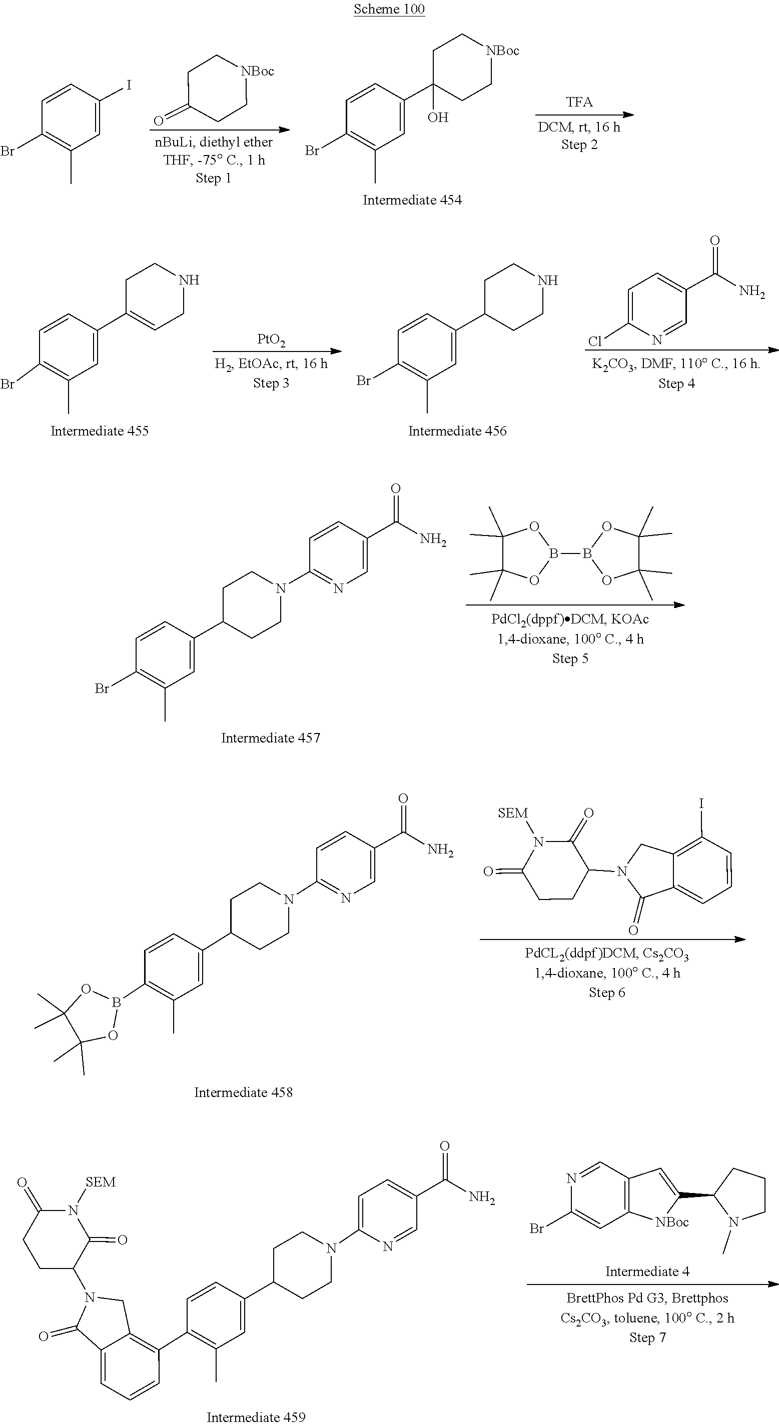

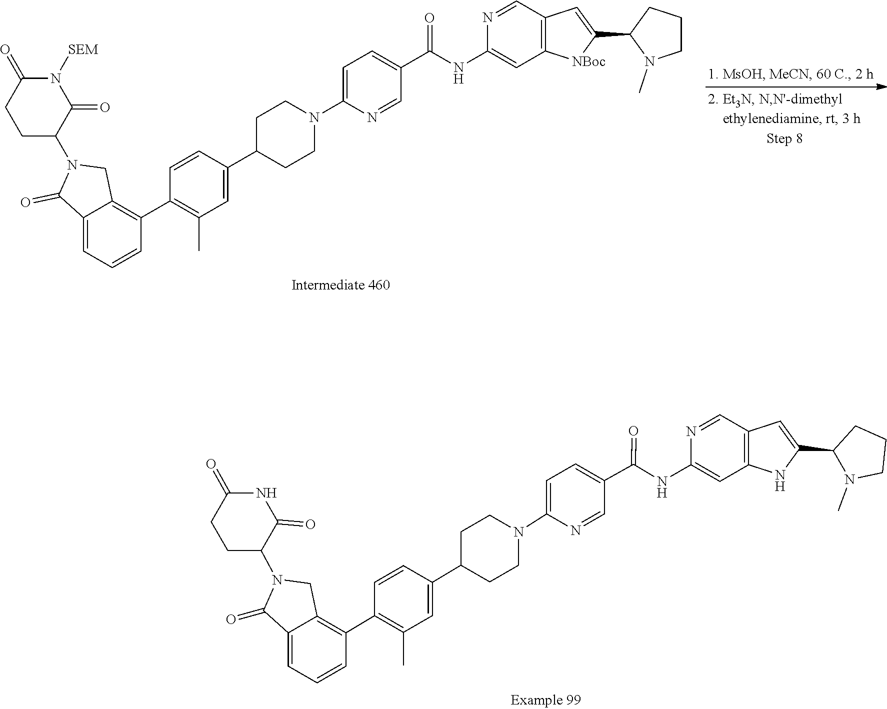 [SMILES: Cc1cc(C2CCN(c3ccc(C(=O)Nc4cc5c(cn4)cc([C@H]4CCCN4C)n5C(=O)OC(C)(C)C)cn3)CC2)ccc1-c1cccc2c1CN(C1CCC(=O)N(COCC[Si](C)(C)C)C1=O)C2=O ; Cc1cc(C2CCN(c3ccc(C(=O)Nc4cc5[nH]c([C@H]6CCCN6C)cc5cn4)cn3)CC2)ccc1-c1cccc2c1CN(C1CCC(=O)NC1=O)C2=O]

### Step 1

### Intermediate 454: tert-butyl 4-(4-bromo-3-methylphenyl)-4-hydroxypiperidine-1-carboxylate

To a cooled (−75° C.) solution of 1-bromo-4-iodo-2-methylbenzene (CAS No. 202865-85-5, 10.0 g, 33.67 mmol) in diethyl ether (100 mL) was added n-butyllithium (22.0 mL, 33.67 mmol) and the reaction mixture was stirred for 15 min. Then tert-butyl 4-oxopiperidine-1-carboxylate (CAS No. 79099-07-3, 7.38 g, 37.04 mmol) in tetrahydrofuran (20 mL) was added at −75° C. and the reaction mixture was stirred at room temperature for 1 h. After completion, the reaction mixture was quenched with saturated aqueous ammonium chloride solution (100 mL). The organic layer was separated, washed with water (100 mL), dried over anhydrous Na₂SO₄, filtered and concentrated in vacuo. The residue was dissolved in n-pentane, cooled to 0° C. and crystallized to afford tert-butyl 4-(4-bromo-3-methylphenyl)-4-hydroxypiperidine-1-carboxylate (Intermediate 454, 4.0 g, 32%) as a white solid. ¹H NMR (400 MHz, CDCl₃) δ ppm 1.47 (s, 9H), 1.59-1.73 (m, 2H), 1.77-2.01 (m, 2H), 2.40 (s, 3H), 3.17-3.21 (m, 2H), 3.67-3.77 (m, 2H), 4.02 (br s, 1H), 7.10-7.15 (m, 1H), 7.33 (s, 1H), 7.46-7.51 (m, 1H). Mass spec m/z 370.2 [M+H]⁺.

### Step 2

### Intermediate 455: 4-(4-bromo-3-methylphenyl)-1,2,3,6-tetrahydropyridine

To a cooled (0° C.) solution of tert-butyl 4-(4-bromo-3-methylphenyl)-4-hydroxypiperidine-1-carboxylate (Intermediate 454, 8.0 g, 9.93 mmol) in dichloromethane (60 mL) was added trifluoroacetic acid (40 mL) and the reaction mixture was stirred at room temperature for 16 h. After completion, the reaction mixture was concentrated in vacuo. The obtained residue was diluted with saturated aqueous sodium bicarbonate solution (200 mL) and extracted with dichloromethane (2×200 mL). The combined organic layers were dried over anhydrous Na₂SO₄ filtered and concentrated in vacuo to afford 4-(4-bromo-3-methylphenyl)-1,2,3,6-tetrahydropyridine (Intermediate 455, 6.0 g) as an off-white solid, which was used for the next step without further purification. ¹H NMR (400 MHz, DMSO-d₆) δ ppm 2.36 (s, 3H), 2.61-2.67 (m, 2H), 3.30 (t, J=6.0 Hz, 2H), 3.71-3.77 (m, 2H), 6.23 (s, 1H), 7.23 (d, J=8.40 Hz, 1H), 7.46 (s, 1H), 7.57 (d, J=8.40 Hz, 1H). Exchangeable proton not observed. Mass spec m/z 254.1 [M+H+2]⁺.

### Step 3

### Intermediate 456: 4-(4-bromo-3-methylphenyl)piperidine

To a solution of 4-(4-bromo-3-methylphenyl)-1,2,3,6-tetrahydropyridine (Intermediate 455, 6.0 g, 22.13 mmol) in ethyl acetate (200 mL) was added platinum (IV) oxide (0.90 g, 3.96 mmol). The reaction mixture was stirred at room temperature for 16 h under 60 psi H₂ atmosphere. After completion, the reaction mixture was filtered through a celite bed, and the filter pad was washed with MeOH (200 mL). The filtrate was concentrated in vacuo to afford 4-(4-bromo-3-methylphenyl)piperidine (Intermediate 456, 5.0 g, 89%) as a gummy liquid, which was used for the next step without further purification. ¹H NMR (400 MHz, DMSO-d₆) δ ppm 1.70-1.82 (m, 2H), 1.86-1.95 (m, 2H), 2.32 (s, 3H), 2.77-2.83 (m, 1H), 2.96-3.04 (m, 2H), 3.33-3.40 (m, 2H), 6.97-7.06 (m, 1H), 7.21 (s, 1H), 7.49-7.55 (m, 1H), 8.68 (br s, 1H). Mass spec m/z 255.9 [M+H+2]⁺.

### Step 4

### Intermediate 457: 6-(4-(4-bromo-3-methylphenyl)piperidin-1-yl)nicotinamide

To a solution of 4-(4-bromo-3-methylphenyl)piperidine (Intermediate 456, 4.0 g, 11.33 mmol) and 6-chloronicotinamide (CAS No. 6271-78-9, 3.0 g, 19.0 mmol) in dimethylformamide (40 mL) was added potassium carbonate (10.0 g, 71.63 mmol). The reaction mixture was stirred at 110° C. for 16 h. After completion, the reaction mixture was poured into water (200 mL) and the resultant precipitate was collected by filtration, washed with water (50 mL) and dried in vacuo to afford 6-(4-(4-bromo-3-methylphenyl)piperidin-1-yl)nicotinamide (Intermediate 457, 3.0 g, 31%) as a yellow solid. ¹H NMR (400 MHz, DMSO-d₆) δ ppm 1.52-1.57 (m, 2H), 1.80-1.85 (m, 2H), 2.31 (s, 3H), 2.76-2.80 (m, 1H), 2.91-2.97 (m, 2H), 4.53-4.57 (m, 2H), 6.87 (d, J=9.12 Hz, 1H), 6.97-7.19 (m, 2H), 7.24 (br s, 1H), 7.47 (d, J=8.40 Hz, 1H), 7.73 (br s, 1H), 7.95 (d, J=8.40 Hz, 1H), 8.61 (s, 1H). Mass spec m/z 374.0 [M+H]⁺.

### Step 5

### Intermediate 458: 6-(4-(3-methyl-4-(4,4,5,5-tetramethyl-1,3,2-dioxaborolan-2-yl)phenyl)piperidin-1-yl)nicotinamide

To a solution of 6-(4-(4-bromo-3-methylphenyl)piperidin-1-yl)nicotinamide (Intermediate 457, 2.0 g, 5.34 mmol) and 4,4,4′,4′,5,5,5′,5′-octamethyl-2,2′-bi-1,3,2-dioxaborolane (CAS No. 73183-34-3, 1.8 g, 16.0 mmol) in 1,4-dioxane (40 mL) was added potassium acetate (1.6 g, 5.2 mmol). The reaction mixture was purged with argon for 15 min then PdCl₂(dppf)·DCM (0.48 g, 0.58 mmol) was added. The reaction mixture was purged with argon for another 10 min and stirred at 100° C. for 4 h. After completion, the reaction mixture was concentrated in vacuo. The resulting crude residue was triturated with diethyl ether (2×20 mL) and dried in vacuo to afford 6-(4-(3-methyl-4-(4,4,5,5-tetramethyl-1,3,2-dioxaborolan-2-yl)phenyl)piperidin-1-yl)nicotinamide (Intermediate 458, 4.0 g, 96%) as a grey solid, which was used for the next step without further purification. Mass spec m/z 422.4 [M+H]⁺.

### Step 6

### Intermediate 459: 6-(4-(4-(2-(2,6-dioxo-1-((2-(trimethylsilyl)ethoxy)methyl)piperidin-3-yl)-1-oxoisoindolin-4-yl)-3-methylphenyl)piperidin-1-yl)nicotinamide

To a solution of 6-(4-(3-methyl-4-(4,4,5,5-tetramethyl-1,3,2-dioxaborolan-2-yl)phenyl)piperidin-1-yl)nicotinamide (Intermediate 458, 3.73 g, 4.78 mmol) and 3-(4-iodo-1-oxoisoindolin-2-yl)-1-((2-(trimethylsilyl)ethoxy)methyl)piperidine-2,6-dione (Intermediate 8, 1.6 g, 2.4 mmol) in 1,4-dioxane (8 mL) was added cesium carbonate (2.10 g, 6.43 mmol). The reaction mixture was purged with argon for 15 min then PdCl₂(dppf) DCM (0.17 g, 0.24 mmol) was added. The reaction mixture was purged with argon for another 10 min and stirred at 100° C. for 4 h. After completion, the reaction mixture was filtered through a celite bed, and the filter pad was washed with ethyl acetate (100 mL). The filtrate was concentrated in vacuo and the crude material was triturated with n-pentane, filtered and dried in vacuo to afford 6-(4-(4-(2-(2,6-dioxo-1-((2-(trimethylsilyl)ethoxy)methyl)piperidin-3-yl)-1-oxoisoindolin-4-yl)-3-methylphenyl)piperidin-1-yl)nicotinamide (Intermediate 459, 1.50 g) as a brown solid. ¹H NMR (400 MHz, DMSO-d₆) δ ppm −0.08 (s, 9H), 0.76-0.83 (m, 2H), 1.61-1.67 (m, 2H), 1.86-1.89 (m, 2H), 1.96-2.02 (m, 1H), 2.07 (s, 3H), 2.30-2.35 (m, 1H), 2.71-2.75 (m, 1H), 2.80-2.87 (m, 1H), 2.94-3.04 (m, 3H), 3.44-3.50 (m, 2H), 4.01-4.04 (m, 1H), 4.19-4.22 (m, 1H), 4.56-4.60 (m, 2H), 4.96-5.07 (m, 2H), 5.23-5.28 (m, 1H), 6.88 (d, J=9.12 Hz, 1H), 7.09 (br s, 1H), 7.13-7.20 (m, 2H), 7.23 (br s, 1H), 7.48 (d, J=7.05 Hz, 1H), 7.60 (t, J=7.60 Hz, 1H), 7.76 (d, J=7.60 Hz, 2H), 7.94-7.97 (m, 1H), 8.62 (s, 1H). Mass spec m/z 668.5 [M+H]⁺.

### Step 7

### Intermediate 460: tert-butyl 6-(6-(4-(4-(2-(2,6-dioxo-1-((2-(trimethylsilyl)ethoxy)methyl)piperidin-3-yl)-1-oxoisoindolin-4-yl)-3-methylphenyl)piperidin-1-yl)nicotinamido)-2-((R)-1-methylpyrrolidin-2-yl)-1H-pyrrolo[3,2-c]pyridine-1-carboxylate

To a solution of 6-(4-(4-(2-(2,6-dioxo-1-((2-(trimethylsilyl) ethoxy)methyl)piperidin-3-yl)-1-oxoisoindolin-4-yl)-3-methylphenyl)piperidin-1-yl)nicotinamide (Intermediate 459, 0.70 g, 1.04 mmol) in toluene (20 mL) was added tert-butyl (R)-6-bromo-2-(1-methylpyrrolidin-2-yl)-1H-pyrrolo[3,2-c]pyridine-1-carboxylate (Intermediate 4, 0.47 g, 1.25 mmol) followed by cesium carbonate (1.03 g, 3.14 mmol). The reaction mixture was purged with argon for 15 min then BrettPhos Pd G3 (0.09 g, 0.10 mmol) and BrettPhos (0.05 g, 0.10 mmol) were added. The reaction mixture was purged with argon for another 10 min and stirred at 100° C. for 2 h. After completion, the reaction mixture was filtered through a celite bed, the filter pad was washed with ethyl acetate (100 mL) and the filtrate concentrated in vacuo. The crude material was triturated with n-pentane, filtered and dried in vacuo to afford tert-butyl 6-(6-(4-(4-(2-(2,6-dioxo-1-((2-(trimethylsilyl)ethoxy)methyl)piperidin-3-yl)-1-oxoisoindolin-4-yl)-3-methylphenyl)piperidin-1-yl)nicotinamido)-2-((R)-1-methylpyrrolidin-2-yl)-1H-pyrrolo[3,2-c]pyridine-1-carboxylate (Intermediate 460, 1.4 g) as a brown solid. Mass spec m/z 967.5 [M+H]⁺.

### Step 8

### Example 99: 6-(4-(4-(2-(2,6-dioxopiperidin-3-yl)-1-oxoisoindolin-4-yl)-3-methylphenyl)piperidin-1-yl)-N-(2-((R)-1-methylpyrrolidin-2-yl)-1H-pyrrolo[3,2-c]pyridin-6-yl)nicotinamide

To a solution of tert-butyl 6-(6-(4-(4-(2-(2,6-dioxo-1-((2-(trimethylsilyl)ethoxy)methyl)piperidin-3-yl)-1-oxoisoindolin-4-yl)-3-methylphenyl)piperidin-1-yl)nicotinamido)-2-((R)-1-methylpyrrolidin-2-yl)-1H-pyrrolo[3,2-c]pyridine-1-carboxylate (Intermediate 460, 0.80 g, 0.82 mmol) in acetonitrile (10 mL) was added methanesulfonic acid (0.54 mL, 8.27 mmol) and the reaction mixture was stirred at 60° C. for 2 h. The reaction mixture was cooled to room temperature, then N,N′-dimethylethylenediamine (0.49 mL, 4.13 mmol) followed by triethylamine (2.32 mL, 16.54 mmol) were added and the mixture was stirred at room temperature for 3 h. The reaction mixture was diluted with water (50 mL) and the resultant precipitate was collected by filtration and dried in vacuo. The crude solid was purified by preparative HPLC (Method B) to afford 6-(4-(4-(2-(2,6-dioxopiperidin-3-yl)-1-oxoisoindolin-4-yl)-3-methylphenyl)piperidin-1-yl)-N-(2-((R)-1-methylpyrrolidin-2-yl)-1H-pyrrolo[3,2-c]pyridin-6-yl)nicotinamide (Example 99, 0.08 g, 14%) as an off-white solid. ¹H NMR (400 MHz, DMSO-d₆) δ ppm 1.64-1.72 (m, 2H), 1.76-1.94 (m, 6H), 2.08 (s, 3H), 2.11-2.15 (m, 1H), 2.16 (s, 3H), 2.24-2.28 (m, 1H), 2.37-2.41 (m, 1H), 2.52-2.55 (m, 1H), 2.81-2.91 (m, 2H), 2.99-3.05 (m, 2H), 3.12-3.17 (m, 1H), 3.27-3.30 (m, 1H), 4.07-4.11 (m, 1H), 4.19-4.24 (m, 1H), 4.61-4.65 (m, 2H), 5.08-5.13 (m, 1H), 6.38 (s, 1H), 6.94 (d, J=9.13 Hz, 1H), 7.15-7.18 (m, 2H), 7.26 (s, 1H), 7.47-7.49 (m, 1H), 7.59 (t, J=7.50 Hz, 1H), 7.73-7.75 (m, 1H), 8.16-8.20 (m, 2H), 8.48 (s, 1H), 8.80-8.84 (m, 1H), 10.27 (br s, 1H), 10.91 (br s, 1H), 11.33 (br s, 1H). Mass spec m/z 737.2 [M+H]⁺.

### Example 100 was Prepared Following Scheme 101

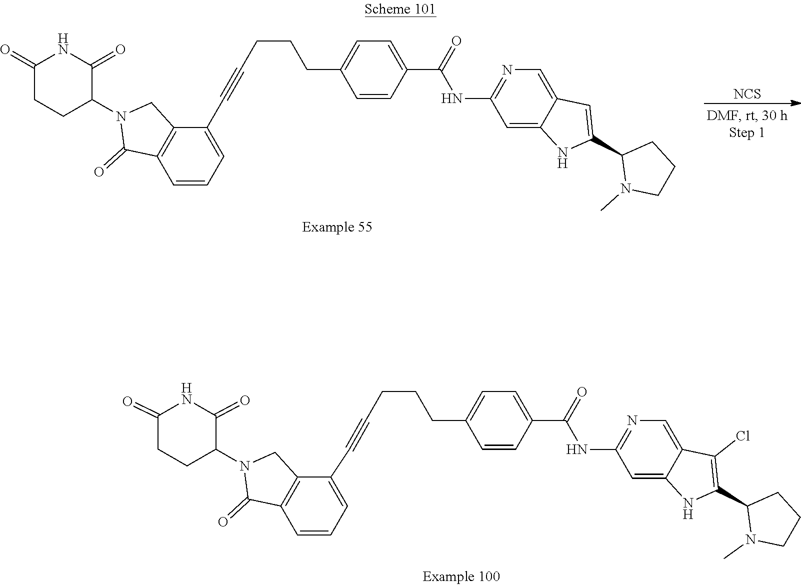 [SMILES: CN1CCC[C@@H]1c1cc2cnc(NC(=O)c3ccc(CCCC#Cc4cccc5c4CN(C4CCC(=O)NC4=O)C5=O)cc3)cc2[nH]1 ; "NCS" ; CN1CCC[C@@H]1c1[nH]c2cc(NC(=O)c3ccc(CCCC#Cc4cccc5c4CN(C4CCC(=O)NC4=O)C5=O)cc3)ncc2c1Cl]

### Step 1

### Example 100: N-(3-chloro-2-((R)-1-methylpyrrolidin-2-yl)-1H-pyrrolo[3,2-c]pyridin-6-yl)-4-(5-(2-(2,6-dioxopiperidin-3-yl)-1-oxoisoindolin-4-yl)pent-4-yn-1-yl)benzamide

To a solution of 4-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide (Example 55, 0.20 g, 0.31 mmol) in dimethylformamide (10 mL) was added N-chlorosuccinimide (0.08 g, 0.63 mmol). The reaction mixture was stirred at room temperature for 30 h. After completion, the reaction mixture was quenched with ice cold water (50 mL), the resultant precipitate was collected by filtration and dried in vacuo to afford the crude compound. The crude material was purified by preparative HPLC (Method A) to afford N-(3-chloro-2-((R)-1-methylpyrrolidin-2-yl)-1H-pyrrolo[3,2-c]pyridin-6-yl)-4-(5-(2-(2,6-dioxopiperidin-3-yl)-1-oxoisoindolin-4-yl)pent-4-yn-1-yl)benzamide (Example 100, 0.01 g, 6%) as an off-white solid. ¹H NMR (400 MHz, DMSO-d₆) δ ppm 1.85-2.04 (m, 7H), 2.16 (s, 3H), 2.17-2.22 (m, 1H), 2.26-2.30 (m, 1H), 2.41-2.46 (m, 1H), 2.55-2.63 (m, 2H), 2.81-2.85 (m, 2H), 2.87-2.96 (m, 1H), 3.15-3.19 (m, 1H), 3.49-3.53 (m, 1H), 4.31-4.39 (m, 1H), 4.46-4.52 (m, 1H), 5.12-5.16 (m, 1H), 7.38 (d, J=8.0 Hz, 2H), 7.53 (t, J=8.0 Hz, 1H), 7.66-7.69 (m, 1H), 7.69-7.74 (m, 1H), 8.00 (d, J=8.0 Hz, 2H), 8.25 (s, 1H), 8.48-8.51 (m, 1H), 10.52 (s, 1H), 11.01 (s, 1H), 11.68 (br s, 1H). Mass spec m/z 663.0 [M+H]⁺.

### Example 101 was Prepared Following Scheme 102

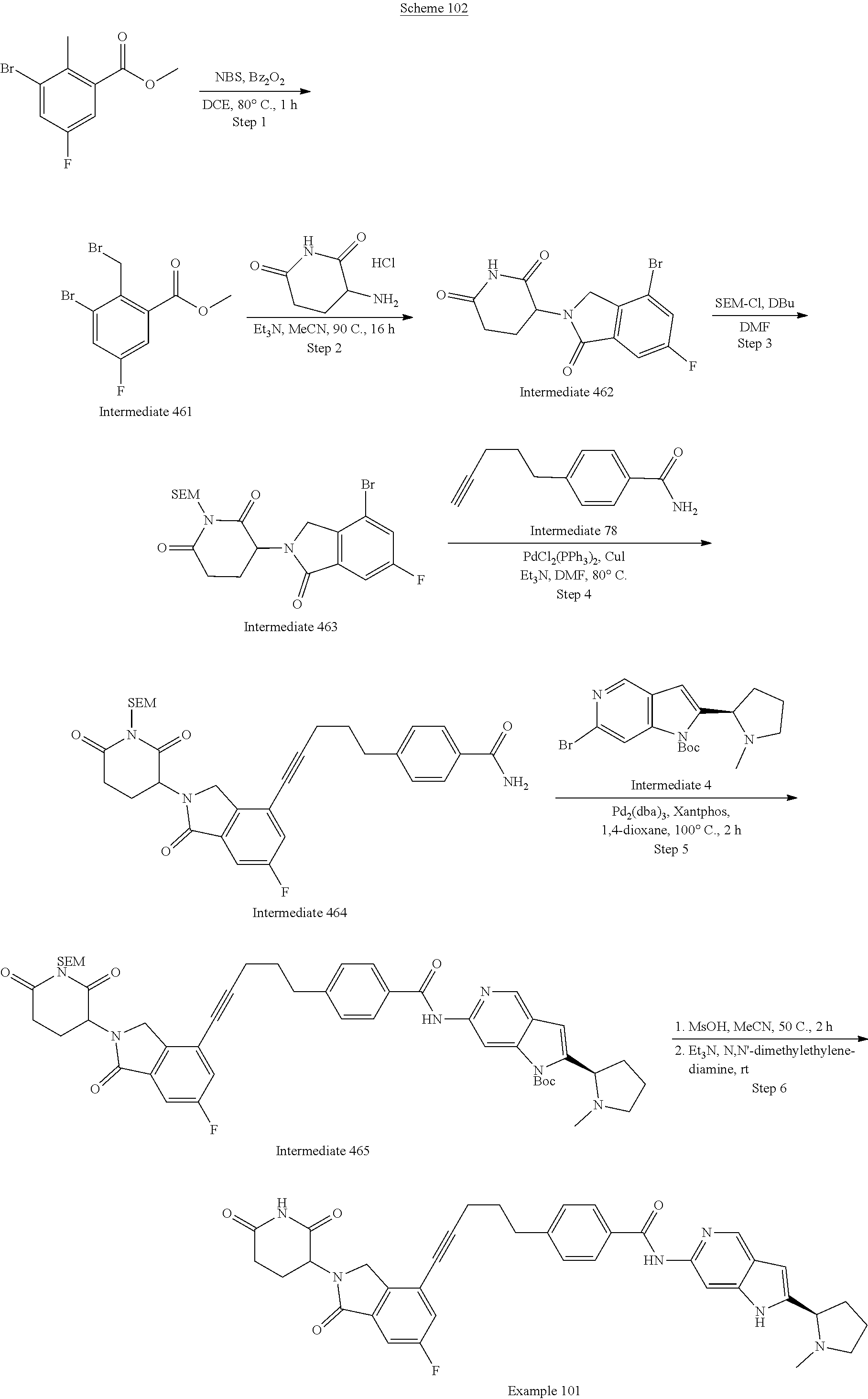

### Step 1

### Intermediate 461: methyl 3-bromo-2-(bromomethyl)-5-fluorobenzoate

To a solution of methyl 3-bromo-5-fluoro-2-methylbenzoate (CAS No. 1805501-44-3, 5.00 g, 20.23 mmol) in 1,2-dichiloroethane (50 mL) was added N-bromosuccinimide (4.04 g, 22.26 mmol) and benzoyl peroxide (0.49 g, 2.02 mmol) and the reaction mixture was heated at 80 ° C. for 1 h. After completion, the reaction mixture was quenched with water (50 mL) and extracted with dichloromethane (50 mL). The combined organic layers were washed with water (50 mL), dried over anhydrous Na₂SO₄, filtered and concentrated in vacuo. The crude material was purified by combi-flash chromatography, by eluting with 20% ethyl acetate in heptane, to afford methyl 3-bromo-2-(bromomethyl)-5-fluorobenzoate (Intermediate 461, 3.50 g, 53%) as a colourless oil.

¹H NMR (400 MHz, DMSO-d₆) δ ppm 3.86 (s, 3H), 4.95 (s, 2H), 7.68 (dd, J=8.92, 2.70 Hz, 1H), 7.94 (dd, J=8.09, 2.70 Hz, 1H).

### Step 2

### Intermediate 462: 3-(4-bromo-6-fluoro-1-oxoisoindolin-2-yl) piperidine-2,6-dione

To a solution of methyl 3-bromo-2-(bromomethyl)-5-fluorobenzoate (Intermediate 461, 3.50 g, 10.7 mmol) and 3-aminopiperidine-2,6-dione hydrochloride (CAS No. 24666-56-6, 2.65 g, 16.1 mmol) in acetonitrile (50 mL) was added triethylamine (3.28 g, 32.2 mmol) and the reaction mixture was heated at 80° C. for 12 h. After completion, the reaction mixture was quenched with water (500 mL) and extracted with dichloromethane (500 mL). The organic layer was separated, washed with water (50 mL), dried over anhydrous Na₂SO₄, filtered and concentrated in vacuo. The crude material was purified by combi-flash chromatography, by eluting with 50% ethyl acetate in heptane, to afford 3-(4-bromo-6-fluoro-1-oxoisoindolin-2-yl) piperidine-2,6-dione (Intermediate 462, 1.5 g, 41%) as a blue solid. ¹H NMR (400 MHz, DMSO-d₆) δ ppm 1.98-2.03 (m, 1H), 2.44-2.48 (m, 1H), 2.57-2.71 (m, 1H), 2.86-2.94 (m, 1H), 4.22-4.27 (m, 1H), 4.38-4.43 (m, 1H), 5.15-5.17 (m, 1H), 7.64 (d, J=8.0 Hz, 1H), 7.91 (d, J=8.0 Hz, 1H), 11.02 (br s, 1H). Mass spec m/z 342.9 [M+H+2]⁺.

### Step 3

### Intermediate 463: 3-(4-bromo-6-fluoro-1-oxoisoindolin-2-yl)-1-((2-(trimethylsilyl)ethoxy)methyl)piperidine-2,6-dione

To a cooled (0° C.) solution of 3-(4-bromo-6-fluoro-1-oxoisoindolin-2-yl)piperidine-2,6-dione (Intermediate 462, 1.5 g, 4.4 mmol) in dimethylformamide (20 mL) was added 1,8-diazabicyclo[5.4.0]undec-7-ene (2.4 mL, 15.0 mmol) and the reaction mixture was stirred for 10 min. 2-(Trimethylsilyl)ethoxymethyl chloride (2.8 mL, 15.0 mmol) was then added and the reaction mixture was stirred at room temperature for 16 h. After completion, the reaction mixture was quenched with water (500 mL) and extracted with ethyl acetate (500 mL). The organic layer was separated, washed with water (500 mL), dried over anhydrous Na₂SO₄, filtered and concentrated in vacuo. The crude material was purified by combi-flash chromatography, by eluting with 50% ethyl acetate in heptane, to afford 3-(4-bromo-6-fluoro-1-oxoisoindolin-2-yl)-1-((2-(trimethylsilyl)ethoxy)methyl)piperidine-2,6-dione (Intermediate 463, 1.1 g, 53%) as an off-white solid. ¹H NMR (400 MHz, DMSO-d₆) δ ppm −0.02 (s, 9H), 0.78-0.87 (m, 2H), 2.05-2.08 (m, 1H), 2.77-2.81 (m, 1H), 3.02-3.06 (m, 1H), 3.52-3.55 (m, 2H), 4.00-4.05 (m, 1H), 4.18-4.23 (m, 1H), 4.40-4.45 (m, 1H), 5.01-5.10 (m, 2H), 5.25-5.30 (m, 1H), 7.65 (d, J=7.04 Hz, 1H), 7.88-7.94 (m, 1H). Mass spec m/z 471.0 [M+H]⁺.

### Step 4

### Intermediate 464: 4-(5-(2-(2,6-dioxo-1-((2-(trimethylsilyl)ethoxy)methyl)piperidin-3-yl)-6-fluoro-1-oxoisoindolin-4-yl)pent-4-yn-1-yl)benzamide

To a solution of 4-(pent-4-yn-1-yl)benzamide (Intermediate 78, 0.79 g, 4.24 mmol) and 3-(4-bromo-6-fluoro-1-oxoisoindolin-2-yl)-1-((2-(trimethylsilyl)ethoxy)methyl)piperidine-2,6-dione (Intermediate 463, 0.80 g, 1.69 mmol) in dimethylformamide (0.5 mL) was added triethylamine (1.66 mL, 11.88 mmol). The reaction mixture was purged with argon for 15 min, then PdCl₂(PPh₃) (0.12 g, 0.16 mmol) and copper(I) iodide (0.03 g, 0.16 mmol) were added. The reaction mixture was further purged with argon for 10 min and heated at 100° C. for 16 h. After completion, the reaction mixture was quenched with water (100 mL) and extracted with ethyl acetate (100 mL). The organic layer was separated, washed with water (50 mL), dried over anhydrous Na₂SO₄, filtered and concentrated in vacuo. The crude material was purified by combi-flash chromatography, by eluting with 50% ethyl acetate in heptane, to afford 4-(5-(2-(2,6-dioxo-1-((2-(trimethylsilyl)ethoxy)methyl)piperidin-3-yl)-6-fluoro-1-oxoisoindolin-4-yl)pent-4-yn-1-yl)benzamide (Intermediate 464, 0.65 g, 66%) as an off-white solid. ¹H NMR (400 MHz, DMSO-d₆) δ ppm −0.04 (s, 9H), 0.81-0.86 (m, 3H), 1.85-1.92 (m, 2H), 2.06-2.08 (m, 1H), 2.39-2.44 (m, 1H), 2.75-2.84 (m, 4H), 3.02-3.08 (m, 1H), 3.49-3.54 (m, 2H), 4.24-4.30 (m, 1H), 4.46-4.51 (m, 1H), 5.01-5.08 (m, 2H), 5.26-5.31 (m, 1H), 7.30-7.35 (m, 2H), 7.57-7.60 (m, 2H), 7.80 (br s, 1H), 7.81-7.85 (m, 2H), 7.91 (br s, 1H). Mass spec m/z 576.2 [M−H]⁻.

### Step 5

### Intermediate 465: tert-butyl 6-(4-(5-(2-(2,6-dioxo-1-((2-(trimethylsilyl) ethoxy)methyl)piperidin-3-yl)-6-fluoro-1-oxoisoindolin-4-yl)pent-4-yn-1-yl)benzamido)-2-((R)-1-methylpyrrolidin-2-yl)-1H-pyrrolo[3,2-c]pyridine-1-carboxylate

To a solution of 4-(5-(2-(2,6-dioxo-1-((2-(trimethylsilyl)ethoxy)methyl)piperidin-3-yl)-6-fluoro-1-oxoisoindolin-4-yl)pent-4-yn-1-yl)benzamide (Intermediate 464, 0.60 g, 1.03 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.47 g, 1.24 mmol) followed by cesium carbonate (1.01 g, 3.11 mmol). The reaction mixture was purged with argon for 15 min, then Pd₂(dba)₃ (0.19 g, 0.20 mmol) and Xantphos (0.24 g, 0.41 mmol) were added. The reaction mixture was further purged with argon for 10 min and heated at 100° C. for 2 h. After completion, the reaction mixture was concentrated in vacuo to obtain the crude compound. The crude material was purified by combi-flash chromatography, by eluting with 80% ethyl acetate in heptane, to afford tert-butyl 6-(4-(5-(2-(2,6-dioxo-1-((2-(trimethylsilyl) ethoxy)methyl)piperidin-3-yl)-6-fluoro-1-oxoisoindolin-4-yl)pent-4-yn-1-yl)benzamido)-2-((R)-1-methylpyrrolidin-2-yl)-1H-pyrrolo[3,2-c]pyridine-1-carboxylate (Intermediate 465, 0.65 g, 71%) as a brown solid. ¹H NMR (400 MHz, DMSO-d₆) δ ppm −0.02 (s, 9H), 0.83-0.88 (m, 2H), 1.75 (s, 9H), 1.78-1.85 (m, 4H), 1.97-2.01 (m, 2H), 2.09-2.16 (m, 1H), 2.41 (s, 3H), 2.43-2.47 (m, 3H), 2.57-2.59 (m, 1H), 2.84-2.91 (m, 3H), 3.07-3.18 (m, 2H), 3.53-3.59 (m, 2H), 3.93-4.00 (m, 1H), 4.30-4.35 (m, 1H), 4.51-4.56 (m, 1H), 5.05-5.13 (m, 2H), 5.31-5.33 (m, 1H), 6.80 (s, 1H), 7.43 (d, J=8.25 Hz, 2H), 7.60-7.67 (m, 2H), 8.05 (d, J=8.25 Hz, 2H), 8.64 (s, 1H), 8.98 (s, 1H), 10.66 (br s, 1H). Mass spec m/z 877.4 [M+H]⁺.

### Step 3

### Example 101: 4-(5-(2-(2,6-dioxopiperidin-3-yl)-6-fluoro-1-oxoisoindolin-4-yl)pent-4-yn-1-yl)-N-(2-((R)-1-methylpyrrolidin-2-yl)-1H-pyrrolo[3,2-c]pyridin-6-yl)benzamide

To a solution of tert-butyl 6-(4-(5-(2-(2,6-dioxo-1-((2-(trimethylsilyl)ethoxy)methyl)piperidin-3-yl)-6-fluoro-1-oxoisoindolin-4-yl)pent-4-yn-1-yl)benzamido)-2-((R)-1-methylpyrrolidin-2-yl)-1H-pyrrolo[3,2-c]pyridine-1-carboxylate (Intermediate 465, 0.80 g, 0.91 mmol) in acetonitrile (15 mL) was added methanesulfonic acid (0.60 mL, 9.12 mmol) and the reaction mixture was heated at 50° C. for 2 h. The reaction mixture was cooled to room temperature, then N,N′-dimethylethylenediamine (0.54 mL, 4.56 mmol) followed by triethylamine (2.56 mL, 18.24 mmol) were added and the mixture was stirred at room temperature for 3 h. The reaction mixture was diluted with water (120 mL) and the resultant precipitate was collected by filtration and dried in vacuo. The crude solid was purified by preparative HPLC (Method A) to afford 4-(5-(2-(2,6-dioxopiperidin-3-yl)-6-fluoro-1-oxoisoindolin-4-yl)pent-4-yn-1-yl)-N-(2-((R)-1-methylpyrrolidin-2-yl)-1H-pyrrolo[3,2-c]pyridin-6-yl)benzamide (Example 101, 0.06 g, 11%) as an off-white solid. ¹H NMR (400 MHz, DMSO-d₆) δ ppm 1.78-1.98 (m, 5H), 1.98-2.07 (m, 1H), 2.16 (s, 3H), 2.17-2.19 (m, 1H), 2.22-2.32 (m, 1H), 2.43-2.48 (m, 2H), 2.52-2.55 (m, 1H), 2.59-2.61 (m, 1H), 2.80-2.96 (m, 3H), 3.12-3.16 (m, 1H), 3.27-3.29 (m, 1H), 4.32-4.38 (m, 1H), 4.45-4.50 (m, 1H), 5.12-5.17 (m, 1H), 6.39 (s, 1H), 7.38 (d, J=8.25 Hz, 2H), 7.54-7.60 (m, 2H), 8.00 (d, J=8.25 Hz, 2H), 8.19 (s, 1H), 8.49 (s, 1H), 10.35 (br s, 1H), 11.03 (br s, 1H), 11.36 (br s, 1H). Mass spec m/z 647.2 [M+H]⁺.

### Examples 102 and 103 were prepared according to Scheme 103

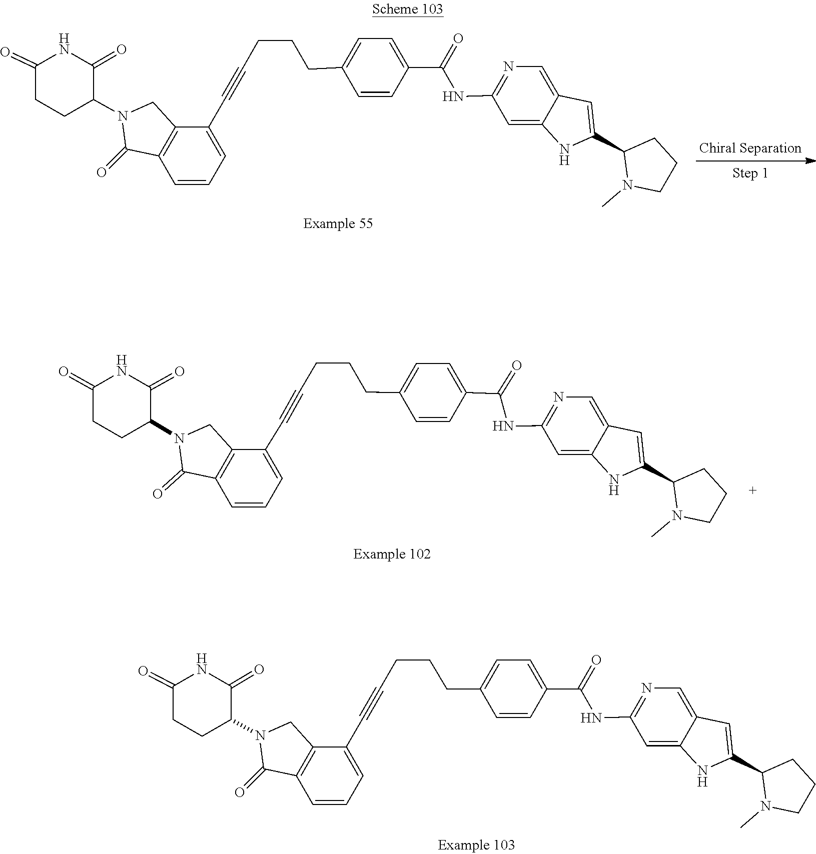 [SMILES: CN1CCC[C@@H]1c1cc2cnc(NC(=O)c3ccc(CCCC#Cc4cccc5c4CN(C4CCC(=O)NC4=O)C5=O)cc3)cc2[nH]1 ; CN1CCC[C@@H]1c1cc2cnc(NC(=O)c3ccc(CCCC#Cc4cccc5c4CN([C@H]4CCC(=O)NC4=O)C5=O)cc3)cc2[nH]1 ; CN1CCC[C@@H]1c1cc2cnc(NC(=O)c3ccc(CCCC#Cc4cccc5c4CN([C@@H]4CCC(=O)NC4=O)C5=O)cc3)cc2[nH]1]

### Step 1

### Example 102: 4-(5-(2-((rel-S)-2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide and Example 103: 4-(5-(2-((rel-R)-2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide

4-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide (Example 55, 0.05 g, 0.07 mmol) was separated by chiral preparative HPLC (Column: RR WHELK (30×250*mm), 5 μm; Mobile Phase A: DCM, Mobile Phase B: IPA; Flow rate: 38 ml/min; isocratic 50% B) to afford 4-(5-(2-((rel-S)-2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide (Example 102, 1st eluting peak, 10 mg, 20%) as a white solid and 4-(5-(2-((rel-R)-2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide (Example 103, 2nd eluting peak, 14 mg, 28%) as a white solid.

### Example 102: 4-(5-(2-((rel-S)-2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide

1^{st} eluting peak from chiral preparative HPLC. Retention time: 5.79 mins.

¹H NMR (400 MHz, DMSO-d₆) δ ppm 1.88-2.03 (m, 6H), 2.16 (s, 3H), 2.18-2.30 (m, 1H), 2.38-2.47 (m, 3H), 2.52-2.56 (m, 2H), 2.56-2.63 (m, 1H), 2.83 (t, J=7.57 Hz, 2H), 2.87-2.95 (m, 1H), 3.12-3.20 (m, 1H), 4.32-4.39 (m, 1H), 4.46-4.52 (m, 1H), 5.15-5.17 (m, 1H), 6.33-6.51 (m, 1H), 7.38 (d, J=8.25 Hz, 2H), 7.51-7.56 (m, 1H), 7.65-7.69 (m, 1H), 7.72 (d, J=7.63 Hz, 1H), 8.00 (d, J=8.25 Hz, 2H), 8.21 (s, 1H), 8.53 (s, 1H), 10.39 (br s, 1H), 11.03 (br s, 1H), 11.39 (br s, 1H). Mass spec m/z 629.0 [M+H]⁺.

### Example 103: 4-(5-(2-((rel-R)-2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide

2nd eluting peak from from chiral preparative HPLC. Retention time: 6.24 mins.

¹H NMR (400 MHz, DMSO-d₆) δ ppm 1.89-2.03 (m, 6H), 2.18 (s, 3H), 2.20-2.25 (m, 1H), 2.44-2.49 (m, 3H), 2.52-2.55 (m, 2H), 2.57-2.63 (m, 1H), 2.83 (t, J=7.57 Hz, 2H), 2.89-2.95 (m, 1H), 3.11-3.20 (m, 1H), 4.32-4.40 (m, 1H), 4.45-4.51 (m, 1H), 5.13-5.17 (m, 1H), 6.42 (s, 1H), 7.38 (d, J=8.25 Hz, 2H), 7.50-7.56 (m, 1H), 7.67 (d, J=6.88 Hz, 1H), 7.72 (d, J=7.50 Hz, 1H), 8.00 (d, J=8.13 Hz, 2H), 8.21 (s, 1H), 8.42 (s, 1H), 10.38 (br s, 1H), 11.02 (br s, 1H), 11.39 (br s, 1H). Mass spec m/z 629.0 [M+H]⁺.

### Examples 104 and 105 were prepared according to Scheme 104

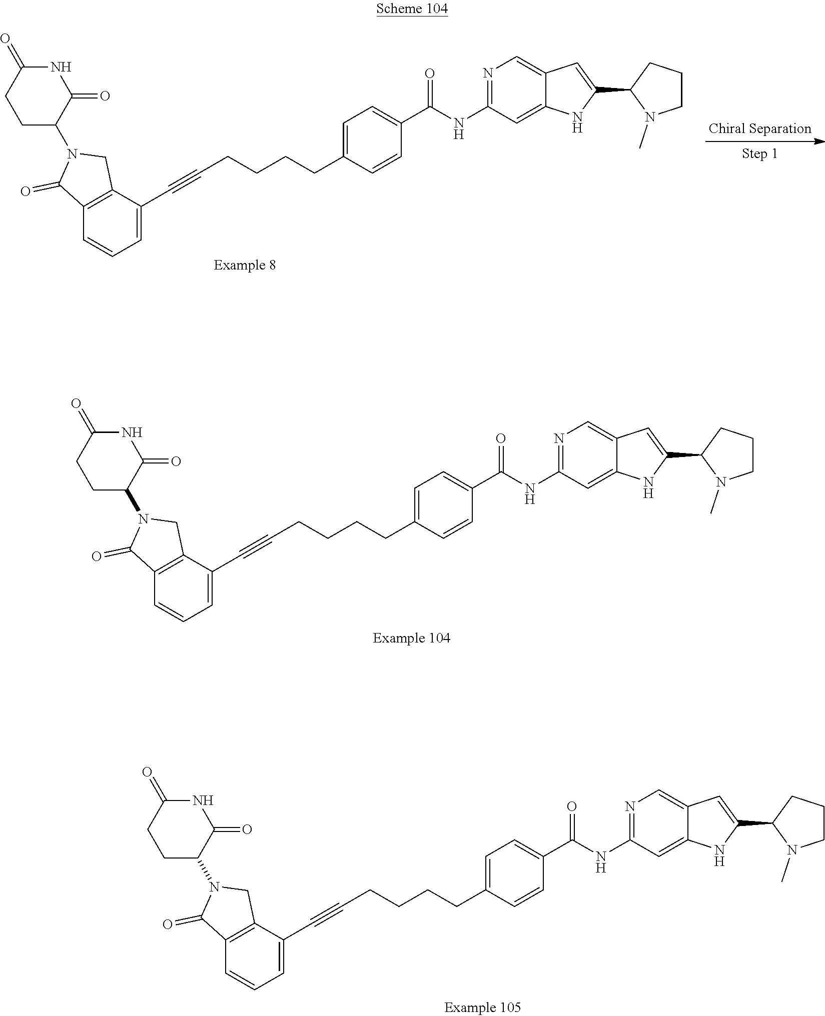 [SMILES: CN1CCC[C@@H]1c1cc2cnc(NC(=O)c3ccc(CCCCC#Cc4cccc5c4CN(C4CCC(=O)NC4=O)C5=O)cc3)cc2[nH]1 ; CN1CCC[C@@H]1c1cc2cnc(NC(=O)c3ccc(CCCCC#Cc4cccc5c4CN([C@H]4CCC(=O)NC4=O)C5=O)cc3)cc2[nH]1 ; CN1CCC[C@@H]1c1cc2cnc(NC(=O)c3ccc(CCCCC#Cc4cccc5c4CN([C@@H]4CCC(=O)NC4=O)C5=O)cc3)cc2[nH]1]

### Step 1

### Example 104: 4-(6-(2-((rel-S)-2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide and Example 105: 4-(6-(2-((rel-R)-2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide

4-(6-(2-(2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide (Example 8, 0.20 g, 0.31 mmol) was separated by chiral preparative HPLC (Column: IK (50×250*mm), 5 m; Mobile Phase A: DCM, Mobile Phase B: iPrOH (1:1); Flow rate: 90 ml/min; isocratic 50% B) to afford 4-(6-(2-((rel-S)-2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide (Example 104, 1st eluting peak, 0.040 g, 9%) as an off-white solid and 4-(6-(2-((rel-R)-2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide (Example 105, 2nd eluting peak, 0.035 g, 8%) as an off-white solid.

### Example 104: 4-(6-(2-((rel-S)-2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide

1^{st} eluting peak from chiral preparative HPLC. Retention time: 4.27 mins.

¹H NMR (400 MHz, DMSO-d₆) δ ppm 1.58-1.66 (m, 2H), 1.75-1.83 (m, 3H), 1.85-1.95 (m, 2H), 1.96-2.05 (m, 1H), 2.12-2.15 (m, 1H), 2.17 (s, 3H), 2.22-2.30 (m, 1H), 2.39-2.46 (m, 2H), 2.53-2.56 (m, 2H), 2.59-2.63 (m, 1H), 2.69-2.75 (m, 2H), 2.85-2.96 (m, 1H), 3.11-3.18 (m, 1H), 4.26-4.35 (m, 1H), 4.40-4.48 (m, 1H), 5.11-5.16 (m, 1H), 6.39 (s, 1H), 7.34 (d, J=8.25 Hz, 2H), 7.49-7.55 (m, 1H), 7.64 (dd, J=7.63, 1.00 Hz, 1H), 7.71 (dd, J=7.57, 0.81 Hz, 1H), 7.98 (d, J=8.38 Hz, 2H), 8.18 (s, 1H), 8.49 (s, 1H), 10.34 (br s, 1H), 11.01 (br s, 1H), 11.36 (br s, 1H). Mass spec m/z 643.4 [M+H]⁺.

### Example 105: 4-(6-(2-((rel-R)-2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide

2nd eluting peak from chiral preparative HPLC. Retention time: 6.16 mins.

¹H NMR (400 MHz, DMSO-d₆) δ ppm 1.58-1.65 (m, 2H), 1.75-1.83 (m, 3H), 1.85-1.95 (m, 2H), 1.96-2.05 (m, 1H), 2.12-2.15 (m, 1H), 2.16 (s, 3H), 2.22-2.30 (m, 1H), 2.39-2.46 (m, 2H), 2.53-2.56 (m, 2H), 2.59-2.63 (m, 1H), 2.69-2.75 (m, 2H), 2.85-2.96 (m, 1H), 3.11-3.16 (m, 1H), 4.28-4.35 (m, 1H), 4.42-4.48 (m, 1H), 5.11-5.16 (m, 1H), 6.38 (s, 1H), 7.34 (d, J=8.25 Hz, 2H), 7.49-7.55 (m, 1H), 7.64 (dd, J=7.63, 1.00 Hz, 1H), 7.71 (dd, J=7.57, 0.81 Hz, 1H), 7.98 (d, J=8.38 Hz, 2H), 8.18 (s, 1H), 8.49 (s, 1H), 10.34 (br s, 1H), 11.01 (br s, 1H), 11.36 (br s, 1H). Mass spec m/z 643.4 [M+H]⁺.

### Example 106 was Prepared Following Scheme 105

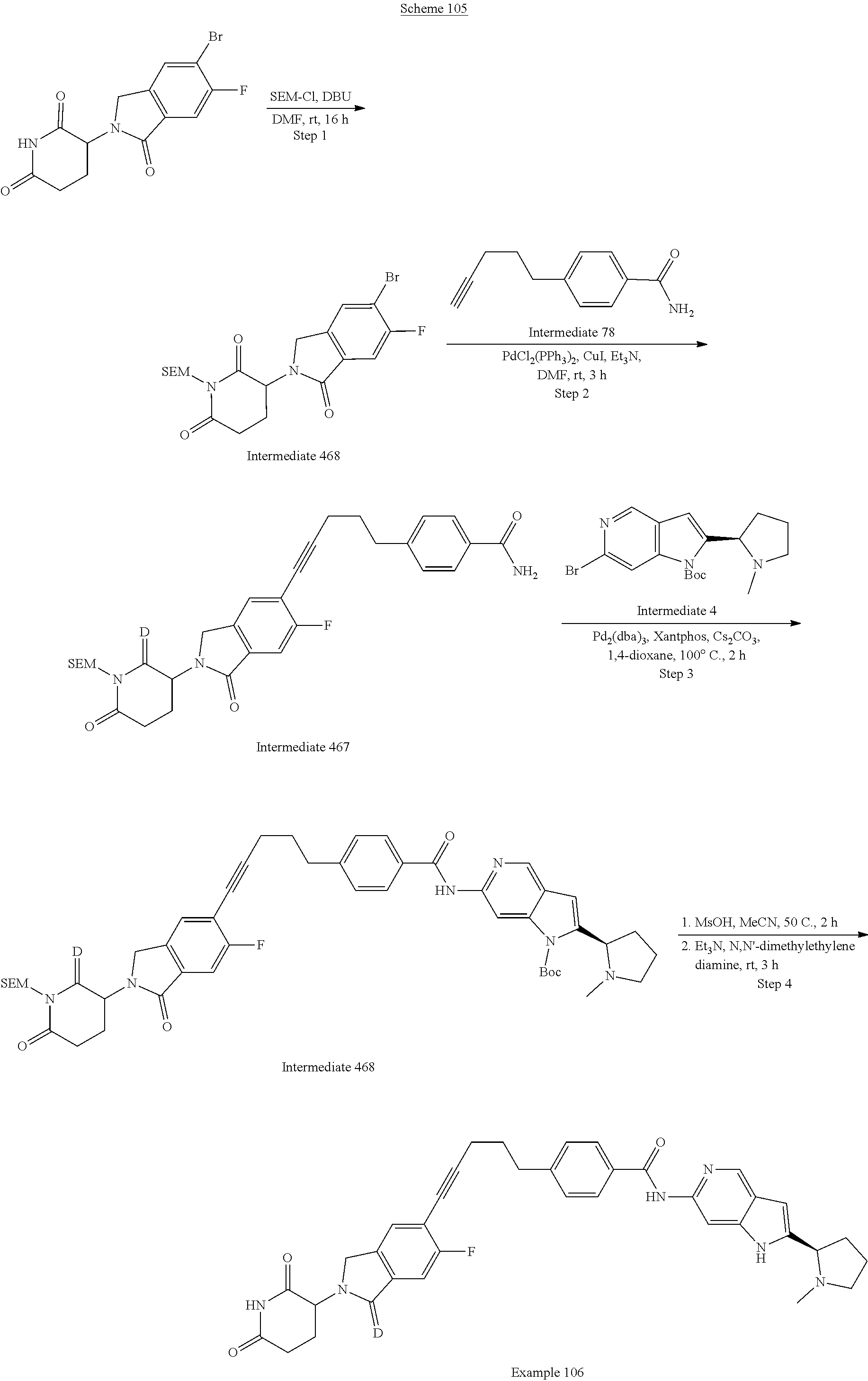

### Step 1

### Intermediate 466: 3-(5-bromo-6-fluoro-1-oxoisoindolin-2-yl)-1-((2-(trimethylsilyl)ethoxy)methyl)piperidine-2,6-dione

To a cooled (0° C.) solution of 3-(5-bromo-6-fluoro-1-oxoisoindolin-2-yl)piperidine-2,6-dione (CAS No. 2409005-96-3, 2.0 g, 5.86 mmol) in dimethylformamide (20 mL) was added 1,8-diazabicyclo[5.4.0]undec-7-ene (3.39 g, 17.6 mmol) and 2-(trimethylsilyl)ethoxymethyl chloride (3.2 mL, 17.6 mmol) and the reaction mixture was stirred at room temperature for 16 h. The reaction mixture was quenched with water (200 mL) and extracted with ethyl acetate (2×100 mL). The combined organic layers were dried over anhydrous Na₂SO₄ and concentrated in vacuo. The crude material was purified by combi-flash chromatography, by eluting with 40% ethyl acetate in heptane, to afford 3-(5-bromo-6-fluoro-1-oxoisoindolin-2-yl)-1-((2-(trimethylsilyl)ethoxy)methyl)piperidine-2,6-dione (Intermediate 466, 1.5 g, 54%) as an off-white solid. ¹H NMR (400 MHz, DMSO-d₆) δ ppm −0.05 (s, 9H), 0.78-0.83 (m, 2H), 2.01-2.07 (m, 1H), 2.30-2.36 (m, 1H), 2.74-2.78 (m, 1H), 2.98-3.07 (m, 1H), 3.46-3.51 (m, 2H), 4.22-4.32 (m, 1H), 4.39-4.47 (m, 1H), 4.98-5.02 (m, 2H), 5.18-5.23 (m, 1H), 7.69 (d, J=7.60 Hz, 1H), 8.03 (d, J=5.60 Hz, 1H). Mass spec m/z 469.0 [M−H]⁻.

### Step 2

### Intermediate 467: 4-(5-(2-(2,6-dioxo-1-((2-(trimethylsilyl)ethoxy)methyl)piperidin-3-yl)-6-fluoro-1-oxoisoindolin-5-yl)pent-4-yn-1-yl)benzamide

To a solution of 4-(pent-4-yn-1-yl)benzamide (Intermediate 78, 0.47 g, 2.54 mmol) and 3-(5-bromo-6-fluoro-1-oxoisoindolin-2-yl)-1-((2-(trimethylsilyl)ethoxy)methyl)piperidine-2,6-dione (Intermediate 466, 0.4 g, 0.84 mmol) in dimethylformamide (15 mL) was added triethylamine (0.95 mL, 6.78 mmol). The reaction mixture was purged with argon for 15 min then copper (I) iodide (0.01 g, 0.08 mmol) and PdCl₂(PPh₃)₂ (0.06 g, 0.08 mmol) were added. The reaction mixture was purged with argon for another 10 min and stirred at room temperature for 3 h. The reaction mixture was diluted with water (200 mL) and extracted with ethyl acetate (3×200 mL). The combined organic layers were dried over anhydrous Na₂SO₄, and concentrated in vacuo. The crude material was purified by combi-flash chromatography, by eluting with 50% ethyl acetate in heptane, to afford 4-(5-(2-(2,6-dioxo-1-((2-(trimethylsilyl)ethoxy)methyl)piperidin-3-yl)-6-fluoro-1-oxoisoindolin-5-yl)pent-4-yn-1-yl)benzamide (Intermediate 467, 0.30 g, 61%) as a yellow solid. ¹H NMR (400 MHz, DMSO-d₆) δ ppm −0.02 (s, 9H), 0.81-0.87 (m, 2H), 1.83-1.90 (m, 2H), 2.02-2.06 (m, 1H), 2.37-2.40 (m, 1H), 2.75-2.85 (m, 3H), 3.01-3.11 (m, 1H), 3.38-3.45 (m, 2H), 3.50-3.58 (m, 2H), 4.25-4.32 (m, 1H), 4.41-4.46 (m, 1H), 5.01-5.10 (m, 2H), 5.20-5.28 (m, 1H), 7.29 (br s, 1H), 7.32 (d, J=6.80 Hz, 2H), 7.61 (d, J=6.80 Hz, 1H), 7.76-7.82 (m, 3H), 7.90 (br s, 1H). Mass spec m/z 576.2 [M−H]⁻.

### Step 3

### Intermediate 468: tert-butyl 6-(4-(5-(2-(2,6-dioxo-1-((2-(trimethylsilyl)ethoxy)methyl)piperidin-3-yl)-6-fluoro-1-oxoisoindolin-5-yl)pent-4-yn-1-yl)benzamido)-2-((R)-1-methylpyrrolidin-2-yl)-1H-pyrrolo[3,2-c]pyridine-1-carboxylate

To a solution of 4-(5-(2-(2,6-dioxo-1-((2-(trimethylsilyl)ethoxy)methyl)piperidin-3-yl)-6-fluoro-1-oxoisoindolin-5-yl)pent-4-yn-1-yl)benzamide (Intermediate 467, 0.30 g, 0.51 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.23 g, 0.62 mmol) followed by cesium carbonate (0.50 g, 1.55 mmol). The reaction mixture was purged with argon for 15 min then Pd₂(dba)₃ (0.07 g, 0.07 mmol) and Xantphos (0.09 g, 0.15 mmol) were added. The reaction mixture was purged with argon for another 10 min and stirred at 100° C. for 2 h. The reaction mixture was filtered through a celite bed, and the filter pad was washed with ethyl acetate (100 mL). The filtrate was concentrated in vacuo and the crude material purified by combi-flash chromatography, by eluting with 5% MeOH in DCM, to afford tert-butyl 6-(4-(5-(2-(2,6-dioxo-1-((2-(trimethylsilyl)ethoxy)methyl)piperidin-3-yl)-6-fluoro-1-oxoisoindolin-5-yl)pent-4-yn-1-yl)benzamido)-2-((R)-1-methylpyrrolidin-2-yl)-1H-pyrrolo[3,2-c]pyridine-1-carboxylate (Intermediate 468, 0.25 g, 55%) as a yellow solid. ¹H NMR (400 MHz, DMSO-d₆) δ ppm −0.02 (s, 9H), 0.83-89 (m, 2H), 1.57-1.63 (m, 2H), 1.69 (s, 9H), 1.74-1.77 (m, 1H), 1.91-1.95 (m, 2H), 2.28-2.34 (m, 3H), 2.36 (s, 3H), 2.55-2.57 (m, 1H), 2.83-2.87 (m, 2H), 2.89-3.18 (m, 3H), 3.34-3.43 (m, 2H), 3.46-3.54 (m, 2H), 3.89-3.94 (m, 1H), 4.23-4.27 (m, 1H), 4.42-4.46 (m, 1H), 4.99-5.06 (m, 2H), 5.21-5.23 (m, 1H), 6.75 (s, 1H), 7.39 (d, J=7.60 Hz, 2H), 7.60 (d, J=8.0 Hz, 1H), 7.73 (d, J=6.40 Hz, 1H), 8.01 (d, J=7.60 Hz, 2H), 8.59 (s, 1H), 8.93 (s, 1H), 10.60 (s, 1H). Mass spec m/z 777.4 [M−99]+.

### Step 4

### Example 106: 4-(5-(2-(2,6-dioxopiperidin-3-yl)-6-fluoro-1-oxoisoindolin-5-yl)pent-4-yn-1-yl)-N-(2-((R)-1-methylpyrrolidin-2-yl)-1H-pyrrolo[3,2-c]pyridin-6-yl)benzamide

To a solution of tert-butyl 6-(4-(5-(2-(2,6-dioxo-1-((2-(trimethylsilyl)ethoxy)methyl)piperidin-3-yl)-6-fluoro-1-oxoisoindolin-5-yl)pent-4-yn-1-yl)benzamido)-2-((R)-1-methylpyrrolidin-2-yl)-1H-pyrrolo[3,2-c]pyridine-1-carboxylate (Intermediate 468, 0.25 g, 0.28 mmol) in acetonitrile (5 mL) was added methanesulfonic acid (0.18 mL, 2.85 mmol) and the reaction mixture was heated at 50° C. for 2 h. The reaction mixture was cooled to room temperature, then N,N′-dimethylethylenediamine (0.17 mL, 1.42 mmol) followed by triethylamine (0.79 mL, 5.70 mmol) were added and the mixture was stirred at room temperature for 3 h. The reaction mixture was concentrated in vacuo and the resultant residue was diluted with water (50 mL). The resultant precipitate was collected by filtration and dried in vacuo. The crude solid was purified by preparative HPLC (Method A) to afford 4-(5-(2-(2,6-dioxopiperidin-3-yl)-6-fluoro-1-oxoisoindolin-5-yl)pent-4-yn-1-yl)-N-(2-((R)-1-methylpyrrolidin-2-yl)-1H-pyrrolo[3,2-c]pyridin-6-yl)benzamide (Example 106, 0.03 g, 19%) as an off-white solid. ¹H NMR (400 MHz, DMSO-d₆) δ ppm 1.78-2.02 (m, 6H), 2.10-2.14 (m, 1H), 2.17 (s, 3H), 2.23-2.27 (m, 1H), 2.31-2.37 (m, 1H), 2.51-2.57 (m, 4H), 2.82-2.90 (m, 3H), 3.10-3.17 (m, 1H), 4.28-4.32 (m, 1H), 4.39-4.43 (m, 1H), 5.08-5.12 (m, 1H), 6.39 (s, 1H), 7.38 (d, J=8.40 Hz, 2H), 7.60 (d, J=8.40 Hz, 1H), 7.71-7.74 (m, 1H), 8.01 (d, J=8.0 Hz, 2H), 8.17-8.21 (m, 1H), 8.49 (s, 1H), 10.37 (br s, 1H), 11.00 (br s, 1H), 11.36 (br s, 1H). Mass spec m/z 647.0 [M+H]⁺.

### Example 107 was Prepared Following Scheme 106

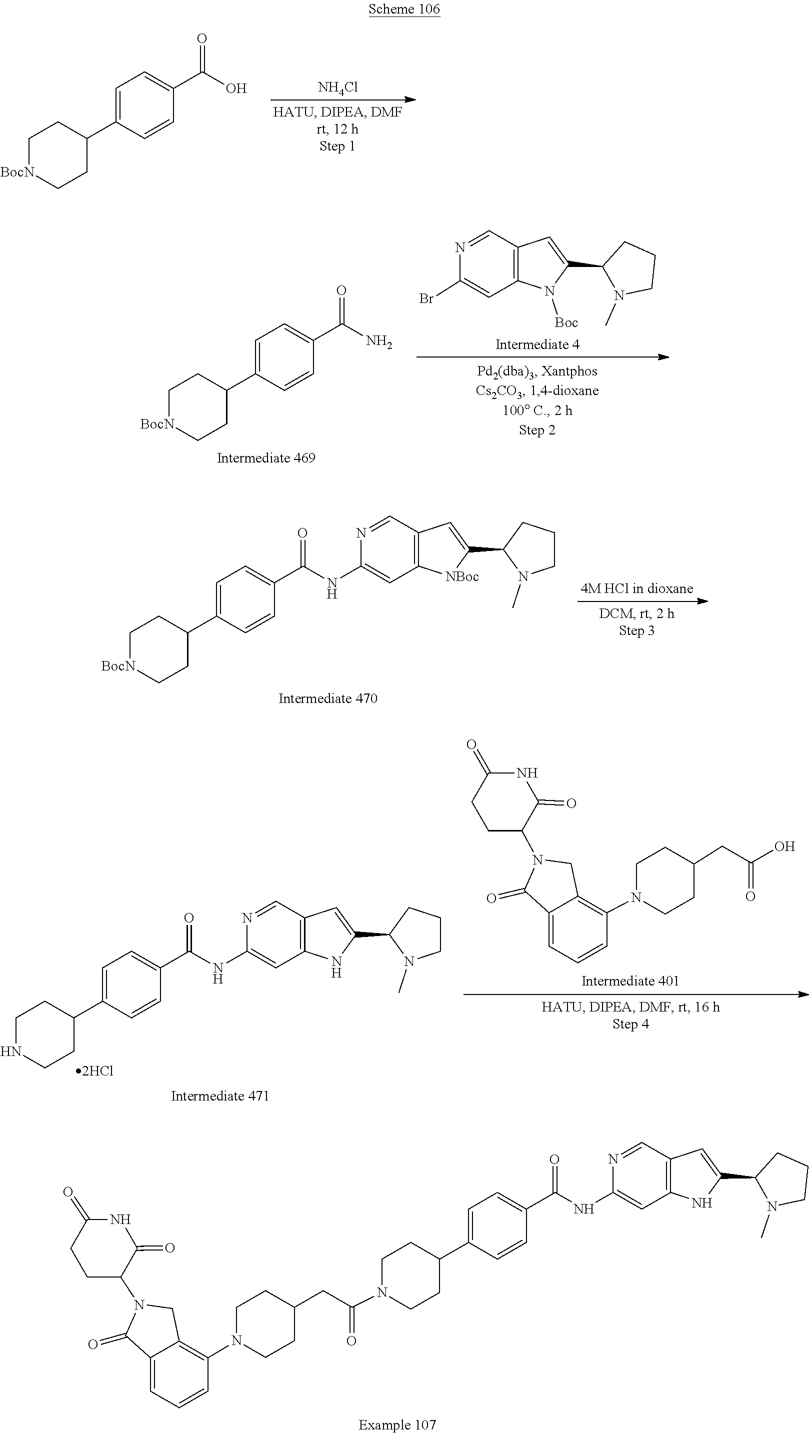

### Step 1

### Intermediate 469: tert-butyl 4-(4-carbamoylphenyl)piperidine-1-carboxylate

To a cooled (0° C.) solution of 4-(1-(tert-butoxycarbonyl)piperidin-4-yl)benzoic acid (CAS No. 149353-75-3, 5.0 g, 16.04 mmol) in dimethylformamide (50 mL) were added HATU (9.15 g, 24.06 mmol) and NN-diisopropylethylamine (10.0 mL, 64.18 mmol). Ammonium chloride (4.29 g, 80.23 mmol) was then added, and the reaction mixture was stirred at room temperature for 12 h. The reaction mixture was poured in ice cold water (500 mL) and stirred for 10 min. The resultant precipitate was collected by filtration and dried in vacuo to afford tert-butyl 4-(4-carbamoylphenyl)piperidine-1-carboxylate (Intermediate 469, 4.3 g, 88%) as an off-white solid, which was used for the next step without further purification. ¹H NMR (400 MHz, DMSO-d₆) δ ppm 1.16-1.28 (m, 1H), 1.41 (s, 9H), 1.46-1.57 (m, 1H), 1.70-1.80 (m, 2H), 2.70-2.86 (m, 3H), 4.05-4.08 (m, 2H), 7.26 (br s, 1H), 7.31 (d, J=7.93 Hz, 2H), 7.80 (d, J=7.93 Hz, 2H), 7.88 (br s, 1H). Mass spec m/z 249.2 [M−56+H]⁺.

### Step 2

### Intermediate 470: tert-butyl (R)-6-(4-(1-(tert-butoxycarbonyl)piperidin-4-yl)benzamido)-2-(1-methylpyrrolidin-2-yl)-1H-pyrrolo[3,2-c]pyridine-1-carboxylate

To a solution of tert-butyl 4-(4-carbamoylphenyl)piperidine-1-carboxylate (Intermediate 469, 1.0 g, 3.15 mmol) in 1,4-dioxane (15 mL) was added tert-butyl(R)-6-bromo-2-(1-methylpyrrolidin-2-yl)-1H-pyrrolo[3,2-c]pyridine-1-carboxylate (Intermediate 4, 1.31 g, 3.46 mmol) followed by cesium carbonate (3.08 g, 9.46 mmol). The reaction mixture was purged with argon for 15 min, then Pd₂(dba)₃ (0.44 g, 0.47 mmol) and Xantphos (0.56 g, 0.94 mmol) were added. The reaction mixture was further purged with argon for 10 min and heated at 100° C. for 2 h then concentrated in vacuo. The crude material was purified by combi-flash chromatography, by eluting with 20% ethyl acetate in heptane, to afford tert-butyl (R)-6-(4-(1-(tert-butoxycarbonyl)piperidin-4-yl)benzamido)-2-(1-methylpyrrolidin-2-yl)-1H-pyrrolo[3,2-c]pyridine-1-carboxylate (Intermediate 470, 1.2 g, 63%) as a yellow solid. ¹H NMR (400 MHz, DMSO-d₆) δ ppm 1.43 (s, 9H), 1.70 (s, 9H), 1.75-1.82 (m, 4H), 1.96-2.02 (m, 2H), 2.27-2.34 (m, 3H), 2.36 (s, 3H), 2.75-2.89 (m, 3H), 3.90-3.97 (m, 1H), 4.01-4.13 (m, 3H), 6.75 (s, 1H), 7.39 (d, J=7.83 Hz, 2H), 7.99 (d, J=7.83 Hz, 2H), 8.59 (s, 1H), 8.93 (s, 1H), 10.61 (br s, 1H). Mass spec m/z 604.3 [M+H]⁺.

### Step 3

### Intermediate 471: (R)—N-(2-(1-methylpyrrolidin-2-yl)-1H-pyrrolo[3,2-c]pyridin-6-yl)-4-(piperidin-4-yl)benzamide dihydrochloride

To a cooled (0° C.) solution of tert-butyl (R)-6-(4-(1-(tert-butoxycarbonyl)piperidin-4-yl)benzamido)-2-(1-methylpyrrolidin-2-yl)-1H-pyrrolo[3,2-c]pyridine-1-carboxylate (Intermediate 470, 1.2 g, 2.0 mmol) in dichloromethane (12 mL) was added 4M hydrochloric acid in 1,4-dioxane (12 mL, 48 mmol) and the reaction mixture was stirred at room temperature for 2 h then concentrated in vacuo. The crude material was triturated with diethyl ether (2×10 mL) and dried in vacuo to afford (R)—N-(2-(1-methylpyrrolidin-2-yl)-1H-pyrrolo[3,2-c]pyridin-6-yl)-4-(piperidin-4-yl)benzamide dihydrochloride (Intermediate 471, 1.0 g, 76%) as a white solid. ¹H NMR (400 MHz, DMSO-d₆) δ ppm 1.56-1.62 (m, 2H), 1.89-2.02 (m, 4H), 2.13-2.24 (m, 2H), 2.81 (s, 3H), 2.98-3.04 (m, 3H), 3.20-3.32 (m, 2H), 3.34-3.41 (m, 2H), 3.69-3.72 (m, 1H), 5.75 (s, 1H), 7.29 (s, 1H), 7.47 (d, J=8.31 Hz, 2H), 8.20 (d, J=7.93 Hz, 2H), 8.28 (s, 1H), 8.95-9.12 (m, 3H), 11.49 (br s, 1H), 12.07 (br s, 1H), 13.32 (br s, 1H).

### Example 107: 4-(1-(2-(1-(2-(2,6-dioxopiperidin-3-yl)-1-oxoisoindolin-4-yl)piperidin-4-yl)acetyl)piperidin-4-yl)-N-(2-((R)-1-methylpyrrolidin-2-yl)-1H-pyrrolo[3,2-c]pyridin-6-yl)benzamide

To a cooled (0° C.) solution of (R)—N-(2-(1-methylpyrrolidin-2-yl)-1H-pyrrolo[3,2-c]pyridin-6-yl)-4-(piperidin-4-yl)benzamide dihydrochloride (Intermediate 471, 0.27 g, 0.62 mmol) in dimethylformamide (4 mL) was added HATU (1.5 g, 3.9 mmol) and N,N-diisopropylethylamine (0.3 mL, 1.55 mmol). 2-(1-(2-(2,6-Dioxopiperidin-3-yl)-1-oxoisoindolin-4-yl)piperidin-4-yl)acetic acid (Intermediate 401, 0.2 g, 0.51 mmol) was added and the reaction mixture stirred at room temperature for 16 h. The reaction mixture was poured into ice cold water (150 mL) and stirred for 10 min. The resultant precipitate was collected by filtration, dried in vacuo and the crude material was purified by preparative HPLC (Method A) to afford 4-(1-(2-(1-(2-(2,6-dioxopiperidin-3-yl)-1-oxoisoindolin-4-yl)piperidin-4-yl)acetyl)piperidin-4-yl)-N-(2-((R)-1-methylpyrrolidin-2-yl)-1H-pyrrolo[3,2-c]pyridin-6-7yl)benzamide (Example 107, 0.08 g, 21%) as an off-white solid. ¹H NMR (400 MHz, DMSO-d₆) δ ppm 1.31-1.65 (m, 4H), 1.76-2.01 (m, 9H), 2.11-2.15 (m, 1H), 2.16 (s, 3H), 2.23-2.31 (m, 1H), 2.33-2.39 (m, 2H), 2.61-2.67 (m, 2H), 2.72-2.80 (m, 2H), 2.86-2.94 (m, 2H), 3.10-3.18 (m, 2H), 3.25-3.30 (m, 2H), 3.37-3.42 (m, 2H), 4.03-4.11 (m, 1H), 4.26-4.33 (m, 1H), 4.39-4.47 (m, 1H), 4.57-4.62 (m, 1H), 5.08-5.13 (m, 1H), 6.38 (s, 1H), 7.18 (d, J=7.95 Hz, 1H), 7.30 (d, J=7.09 Hz, 1H), 7.38 (d, J=8.19 Hz, 2H), 7.40-7.46 (m, 1H), 8.00 (d, J=8.19 Hz, 2H), 8.19 (s, 1H), 8.49 (s, 1H), 10.38 (br s, 1H), 10.98 (br s, 1H), 11.36 (br s, 1H). Mass spec m/z 771.2 [M+H]⁺.

### Example 108 was Prepared Following Scheme 107

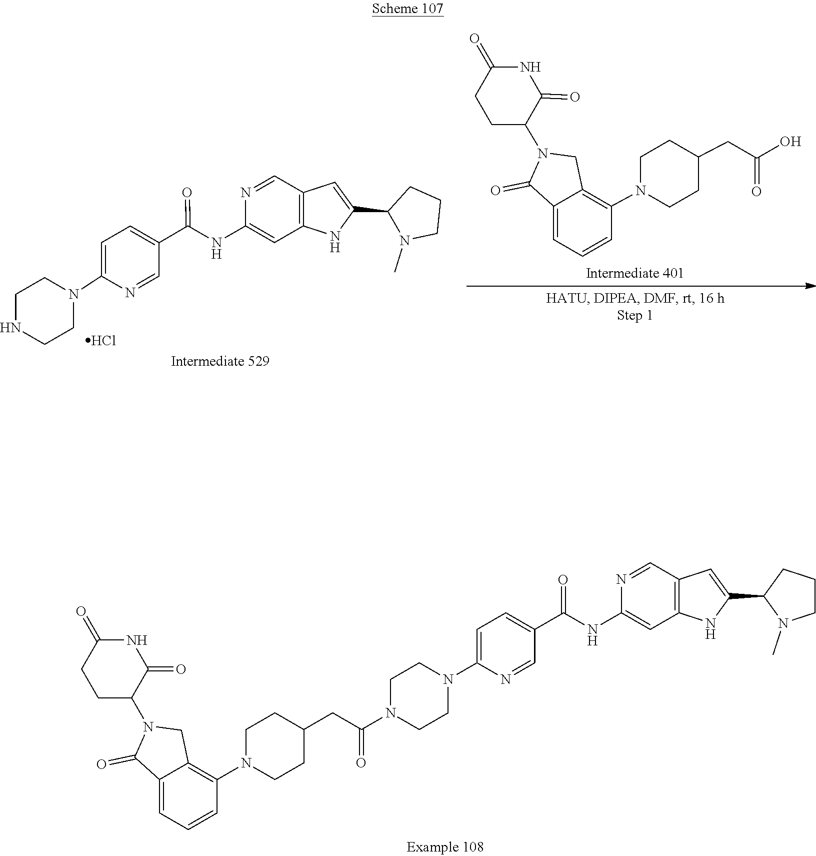 [SMILES: CN1CCC[C@@H]1c1cc2cnc(NC(=O)c3ccc(N4CCNCC4)nc3)cc2[nH]1 ; Cl ; O=C(O)CC1CCN(c2cccc3c2CN(C2CCC(=O)NC2=O)C3=O)CC1 ; CN1CCC[C@@H]1c1cc2cnc(NC(=O)c3ccc(N4CCN(C(=O)CC5CCN(c6cccc7c6CN(C6CCC(=O)NC6=O)C7=O)CC5)CC4)nc3)cc2[nH]1]

### Step 1

### Example 108: 6-(4-(2-(1-(2-(2,6-dioxopiperidin-3-yl)-1-oxoisoindolin-4-yl)piperidin-4-yl)acetyl)piperazin-1-yl)-N-(2-((R)-1-methylpyrrolidin-2-yl)-1H-pyrrolo[3,2-c]pyridin-6-yl)nicotinamide

To a cooled (0° C.) solution of 2-(1-(2-(2,6-dioxopiperidin-3-yl)-1-oxoisoindolin-4-yl)piperidin-4-yl)acetic acid (Intermediate 401, 0.20 g, 0.51 mmol) in dimethylformamide (4 mL) were added HATU (0.29 g, 0.77 mmol) and N,N-diisopropylethylamine (0.5 mL, 2.59 mmol). Then (R)—N-(2-(1-methylpyrrolidin-2-yl)-1H-pyrrolo[3,2-c]pyridin-6-yl)-6-(piperazin-1-yl)nicotinamide hydrochloride (Intermediate 529, 0.34 g, 0.77 mmol) was added and the reaction mixture was stirred at room temperature for 16 h. The reaction mixture was poured into ice cold water (150 mL) and stirred for 10 min, the resultant precipitate was collected by filtration and dried in vacuo. The crude material was purified by preparative HPLC (Method A) to afford 6-(4-(2-(1-(2-(2,6-dioxopiperidin-3-yl)-1-oxoisoindolin-4-yl)piperidin-4-yl)acetyl)piperazin-1-yl)-N-(2-((R)-1-methylpyrrolidin-2-yl)-1H-pyrrolo[3,2-c]pyridin-6-yl)nicotinamide (Example 108, 0.08 g, 21%) as an off-white solid. ¹H NMR (400 MHz, DMSO-d₆) δ ppm 1.36-1.41 (m, 2H), 1.74-1.96 (m, 6H), 1.96-2.04 (m, 1H), 2.11-2.15 (m, 1H), 2.17 (s, 3H), 2.23-2.29 (m, 1H), 2.36-2.39 (m, 2H), 2.45-2.47 (m, 1H), 2.55-2.63 (m, 2H), 2.70-2.81 (m, 2H), 2.86-2.93 (m, 1H), 3.11-3.14 (m, 1H), 3.36-3.42 (m, 2H), 362-3.70 (m, 8H), 4.27-4.31 (m, 1H), 4.40-4.47 (m, 1H), 5.08-5.13 (m, 1H), 6.37 (s, 1H), 6.90 (d, J=9.13 Hz, 1H), 7.17 (d, J=7.88 Hz, 1H), 7.29 (d, J=7.38 Hz, 1H), 7.38-7.46 (m, 1H), 8.14-8.23 (m, 2H), 8.48 (s, 1H), 8.82 (s, 1H), 10.30 (br s, 1H), 10.96 (br s, 1H), 11.33 (br s, 1H). Mass spec m/z 773.0 [M+H]⁺.

### Example 109 was Prepared Following Scheme 107

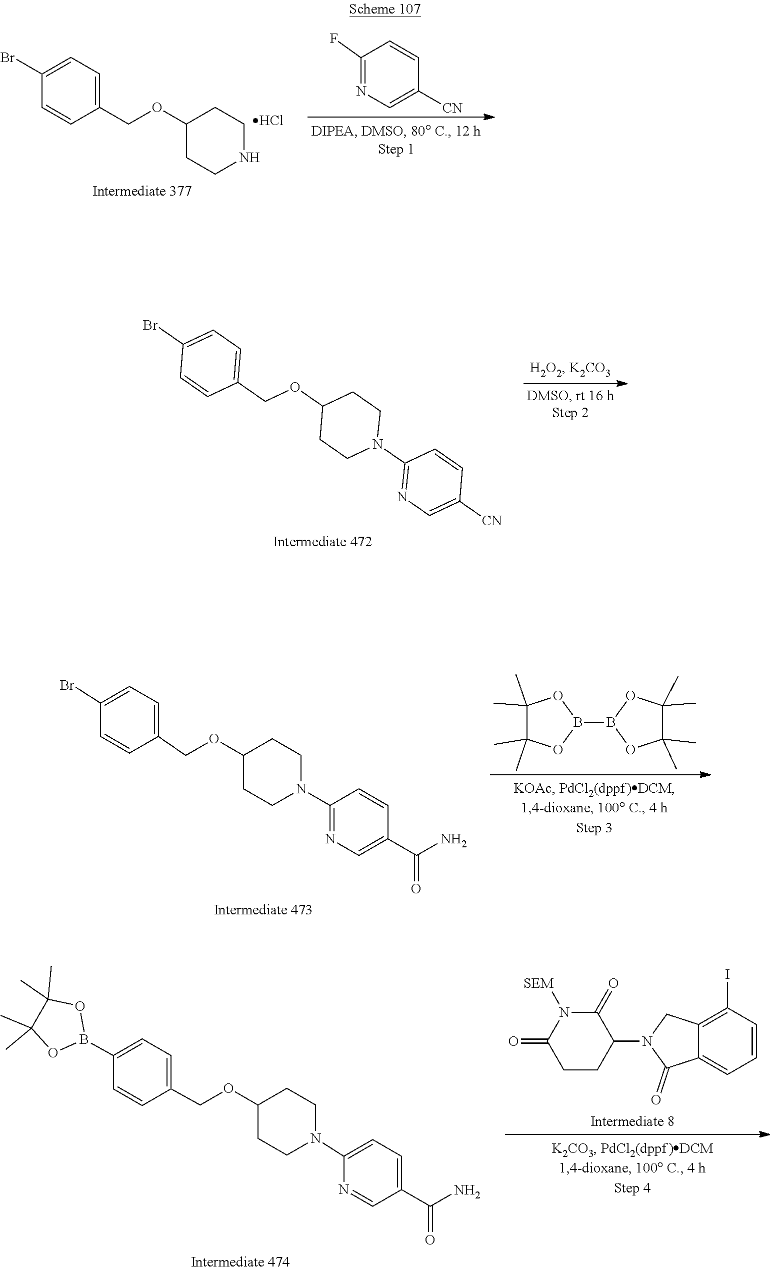

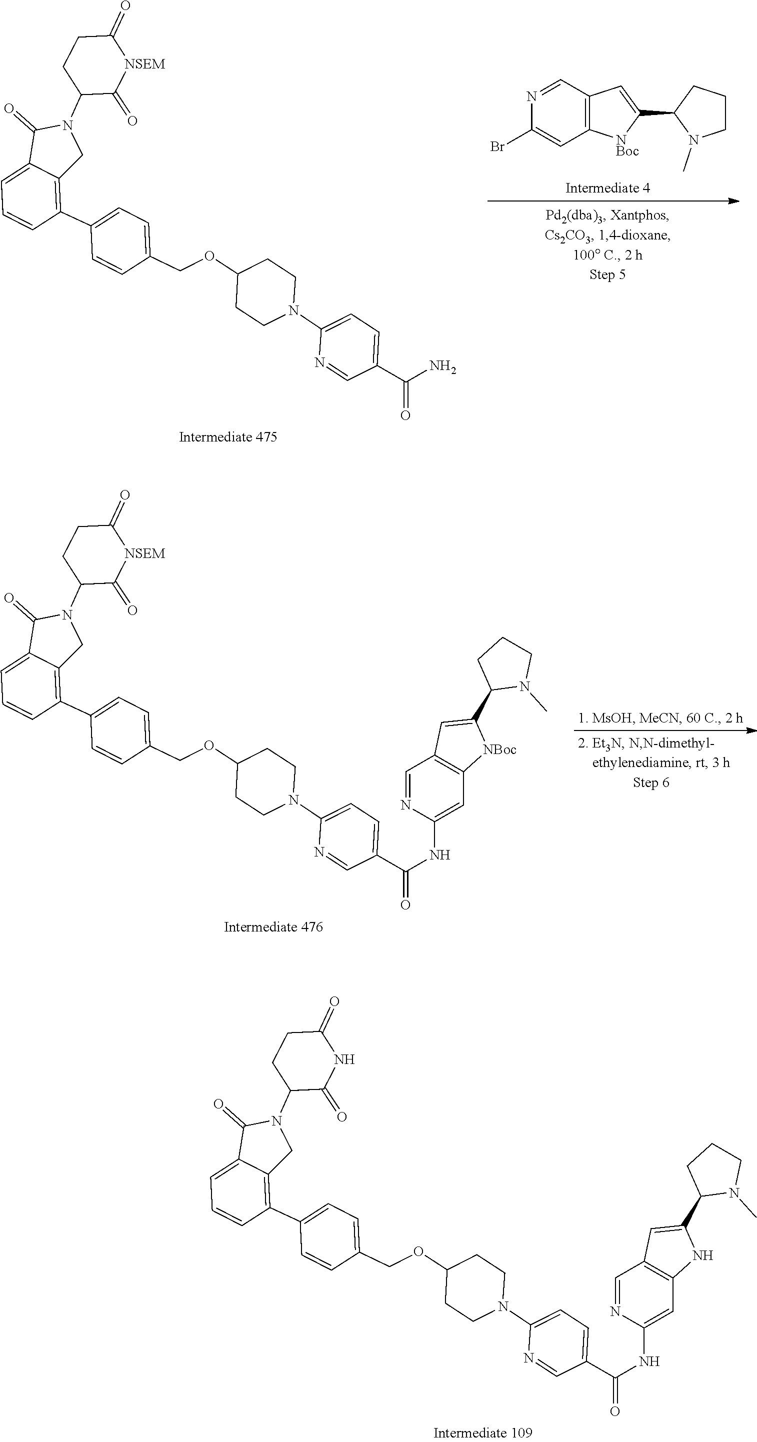 [SMILES: C[Si](C)(C)CCOCN1C(=O)CCC(N2Cc3c(cccc3-c3ccc(COC4CCN(c5ccc(C(N)=O)cn5)CC4)cc3)C2=O)C1=O ; CN1CCC[C@@H]1c1cc2cnc(Br)cc2n1C(=O)OC(C)(C)C ; CN1CCC[C@@H]1c1cc2cnc(NC(=O)c3ccc(N4CCC(OCc5ccc(-c6cccc7c6CN(C6CCC(=O)N(COCC[Si](C)(C)C)C6=O)C7=O)cc5)CC4)nc3)cc2n1C(=O)OC(C)(C)C ; CN1CCC[C@@H]1c1cc2cnc(NC(=O)c3ccc(N4CCC(OCc5ccc(-c6cccc7c6CN(C6CCC(=O)NC6=O)C7=O)cc5)CC4)nc3)cc2[nH]1]

### Step 1

### Intermediate 472: 6-(4-((4-bromobenzyl)oxy)piperidin-1-yl)nicotinonitrile

To a solution of 4-((4-bromobenzyl)oxy)piperidine hydrogen chloride (Intermediate 377, 7.0 g, 22.8 mmol) in dimethyl sulfoxide (50.0 mL) was added 6-fluoronicotinonitrile (CAS No. 3939-12-6, 4.74 g, 38.8 mmol) followed by N,N-diisopropylethylamine and the reaction mixture was stirred at 80° C. for 12 h. The reaction mixture was diluted with ice cold water (250 mL) and extracted with DCM (2×250 mL). The combined organic layers were dried over Na₂SO₄ and concentrated in vacuo. The crude compound was purified through combi-flash chromatography, by eluting with 50% ethyl acetate in heptane, to afford 6-(4-((4-bromobenzyl)oxy)piperidin-1-yl)nicotinonitrile (Intermediate 472, 5.2 g, 61%) as an off-white solid. ¹H NMR (400 MHz, DMSO-d₆) δ ppm 1.44-1.56 (m, 2H), 1.86-1.92 (m, 2H), 3.32-3.36 (m, 2H), 3.64-3.68 (m, 1H), 3.99-4.01 (m, 2H), 4.52 (s, 2H), 6.94 (d, J=8.80 Hz, 1H), 7.31 (d, J=8.40 Hz, 2H), 7.53 (d, J=8.0 Hz, 2H), 8.0 (dd, J=2.40, 9.20 Hz, 1H), 8.46 (d, J=1.20 Hz, 1H). Mass spec m/z 373.9 [M+H+2]⁺.

### Step 2

### Intermediate 473: 6-(4-((4-bromobenzyl)oxy)piperidin-1-yl)nicotinamide

To a solution of 6-(4-((4-bromobenzyl)oxy)piperidin-1-yl)nicotinonitrile (Intermediate 472, 4.3 g, 12.0 mmol) in dimethyl sulfoxide (100 mL) was added potassium carbonate (2.40 g, 17.0 mmol) followed by 30% hydrogen peroxide (1.80 mL, 13 mmol). The reaction mixture was stirred at room temperature for 16 h then quenched with ice-cold water (100 mL). The resultant precipitate was collected by filtration, washed with diethyl ether (100 mL) followed by n-pentane (100 mL), and dried in vacuo to afford 6-(4-((4-bromobenzyl)oxy)piperidin-1-yl)nicotinamide (Intermediate 473, 2.9 g, 64%) as an off-white solid. ¹H NMR (400 MHz, DMSO-d₆) δ ppm 1.42-1.55 (m, 2H), 1.83-1.97 (m, 2H), 3.26-3.34 (m, 2H), 3.63-3.67 (m, 1H), 4.01-4.04 (m, 2H), 4.52 (s, 2H), 6.85 (d, J=9.20 Hz, 1H), 7.10 (br s, 1H), 7.31 (d, J=8.40 Hz, 2H), 7.53 (d, J=8.40 Hz, 2H), 7.73 (br s, 1H), 7.95 (d, J=2.4, 9.20 Hz, 1H), 8.60 (s, 1H). Mass spec m/z 392.2 [M+H+2]⁺.

### Step 3

### Intermediate 474: 6-(4-((4-(4,4,5,5-tetramethyl-1,3,2-dioxaborolan-2-yl)benzyl)oxy)piperidin-1-yl)nicotinamide

To a mixture of 6-(4-((4-bromobenzyl)oxy)piperidin-1-yl)nicotinamide (Intermediate 473, 1.0 g, 2.60 mmol) and 4,4,4′,4′,5,5,5′,5′-octamethyl-2,2′-bi-1,3,2-dioxaborolane (CAS No. 73183-34-3, 0.84 g, 3.30 mmol) in 1,4-dioxane (100 mL) was added potassium carbonate (1.06 g, 7.70 mmol). The reaction mixture was degassed with argon for 15 min, then PdCl₂(dppf)·DCM (0.31 g, 0.38 mmol) was added. The reaction mixture was further purged with argon for another 10 min and heated at 100° C. for 4 h. The reaction mixture was concentrated in vacuo, the crude residue was triturated with diethyl ether (2×50 mL) and dried in vacuo to afford 6-(4-((4-(4,4,5,5-tetramethyl-1,3,2-dioxaborolan-2-yl)benzyl)oxy)piperidin-1-yl)nicotinamide (Intermediate 474, 1.8 g) as a grey solid, which was used in the next step without purification.

Mass spec m/z 438.2 [M+H]⁺.

### Step 4

### Intermediate 475: 6-(4-((4-(2-(2,6-dioxo-1-((2-(trimethylsilyl)ethoxy)methyl)piperidin-3-yl)-1-oxoisoindolin-4-yl)benzyl)oxy)piperidin-1-yl)nicotinamide

To a solution of 6-(4-((4-(4,4,5,5-tetramethyl-1,3,2-dioxaborolan-2-yl)benzyl)oxy)piperidin-1-yl)nicotinamide (Intermediate 474, 1.60 g, 3.66 mmol) in 1,4-dioxane (15 mL) was added 3-(4-iodo-1-oxoisoindolin-2-yl)-1-((2-(trimethylsilyl)ethoxy)methyl)piperidine-2,6-dione (Intermediate 8, 2.57 g, 5.49 mmol) followed by potassium carbonate (2.75 g, 10.29 mmol).

The reaction mixture was degassed with argon for 15 min, then PdCl₂(dppf)·DCM (0.30 g, 0.37 mmol) was added. The reaction mixture was purged with argon for another 10 min and heated at 100° C. for 4 h. The reaction, the reaction mixture was concentrated in vacuo to obtain the crude residue which was purified through combi-flash chromatography, by eluting with 3% methanol in DCM, to afford 6-(4-((4-(2-(2,6-dioxo-1-((2-(trimethylsilyl)ethoxy)methyl)piperidin-3-yl)-1-oxoisoindolin-4-yl)benzyl)oxy)piperidin-1-yl)nicotinamide (Intermediate 475, 1.23 g, 49%) as a yellow solid. ¹H NMR (400 MHz, DMSO-d₆) δ ppm −0.07 (s, 9H), 0.72-0.90 (m, 2H), 1.44-1.61 (m, 2H), 1.94-2.05 (m, 3H), 2.33-2.42 (m, 2H), 2.52-2.55 (m, 2H), 2.75-2.79 (m, 1H), 3.02-3.10 (m, 1H), 3.35-3.45 (m, 2H), 3.47-3.55 (m, 2H), 3.67-3.72 (m, 1H), 4.02-4.05 (m, 2H), 4.31-4.36 (m, 1H), 4.62 (s, 2H), 4.64-4.67 (m, 1H), 5.26-5.31 (m, 1H), 6.86 (d, J=8.80 Hz, 1H), 7.10 (br s, 1H), 7.48 (d, J=8.0 Hz, 2H), 7.57 (d, J=8.0 Hz, 2H), 7.65 (t, J=8.40 Hz, 1H), 7.71 (d, J=7.60 Hz, 1H), 7.77 (d, J=7.20 Hz, 1H), 7.94 (d, J=9.20 Hz, 1H), 8.60 (s, 1H). Mass spec m/z 684.4 [M+H]⁺.

### Step 5

### Intermediate 476: tert-butyl 6-(6-(4-((4-(2-(2,6-dioxo-1-((2-(trimethylsilyl)ethoxy)methyl)piperidin-3-yl)-1-oxoisoindolin-4-yl)benzyl)oxy)piperidin-1-yl)nicotinamido)-2-((R)-1-methylpyrrolidin-2-yl)-1H-pyrrolo[3,2-c]pyridine-1-carboxylate

To a solution of 6-(4-((4-(2-(2,6-dioxo-1-((2-(trimethylsilyl)ethoxy)methyl)piperidin-3-yl)-1-oxoisoindolin-4-yl)benzyl)oxy)piperidin-1-yl)nicotinamide (Intermediate 475, 0.4 g, 0.58 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.27 g, 0.70 mmol) followed by cesium carbonate (0.57 g, 1.76 mmol). The reaction mixture was purged with argon for 15 min then Pd₂(dba)₃ (0.11 g, 0.12 mmol) and Xantphos (0.14 g, 0.23 mmol) were added. The reaction mixture was purged with argon for another 10 min and then stirred at 100° C. for 2 h. The reaction mixture was concentrated in vacuo and the crude material was purified by combi-flash chromatography, by eluting with 80% ethyl acetate in heptane, to afford tert-butyl 6-(6-(4-((4-(2-(2,6-dioxo-1-((2-(trimethylsilyl)ethoxy)methyl)piperidin-3-yl)-1-oxoisoindolin-4-yl)benzyl)oxy)piperidin-1-yl)nicotinamido)-2-((R)-1-methylpyrrolidin-2-yl)-1H-pyrrolo[3,2-c]pyridine-1-carboxylate (Intermediate 476, 0.3 g, 30%) as a yellow solid. ¹H NMR (400 MHz, DMSO-d₆) δ ppm −0.09 (s, 9H), 0.83-0.92 (m, 2H), 1.60-1.70 (m, 4H), 1.72-1.74 (m, 1H), 1.76 (s, 9H), 1.80-1.84 (m, 2H), 1.98-2.04 (m, 3H), 2.37-2.39 (m, 1H), 2.42 (s, 3H), 2.44-2.47 (m, 2H), 2.83-2.86 (m, 1H), 3.12-3.19 (m, 2H), 3.44-3.50 (m, 2H), 3.54-3.58 (m, 2H), 3.79-3.81 (m, 1H), 3.95-4.01 (m, 1H), 4.10-4.18 (m, 2H), 4.40-4.43 (m, 1H), 4.71 (s, 2H), 4.73-4.76 (m, 1H), 5.85-5.89 (m, 1H), 6.81 (s, 1H), 6.99 (d, J=9.20 Hz, 1H), 7.55 (d, J=8.0 Hz, 2H), 7.65 (d, J=8.80 Hz, 2H), 7.73 (t, J=8.40 Hz, 1H), 7.77 (d, J=6.40 Hz, 1H), 7.85 (d, J=7.20 Hz, 1H), 8.22 (dd, J=2.80, 9.20 Hz, 1H), 8.64 (s, 1H), 8.86 (d, J=2.40 Hz, 1H), 8.99 (s, 1H), 10.59 (s, 1H). Mass spec m/z 983.4 [M+H]⁺.

### Step 6

### Example 109: 6-(4-((4-(2-(2,6-dioxopiperidin-3-yl)-1-oxoisoindolin-4-yl)benzyl)oxy)piperidin-1-yl)-N-(2-((R)-1-methylpyrrolidin-2-yl)-1H-pyrrolo[3,2-c]pyridin-6-yl)nicotinamide

To a solution of tert-butyl 6-(6-(4-((4-(2-(2,6-dioxo-1-((2-(trimethylsilyl)ethoxy)methyl)piperidin-3-yl)-1-oxoisoindolin-4-yl)benzyl)oxy)piperidin-1-yl)nicotinamido)-2-((R)-1-methylpyrrolidin-2-yl)-1H-pyrrolo[3,2-c]pyridine-1-carboxylate (Intermediate 476, 0.3 g, 0.31 mmol) in acetonitrile (15 mL) was added methanesulfonic acid (0.3 g, 3.05 mmol) and the reaction mixture was stirred at 50° C. for 2 h. The reaction mixture was cooled to room temperature, then N,N′-dimethylethylenediamine (0.14 g, 1.53 mmol) followed by triethylamine (0.62 g, 6.10 mmol) were added and the mixture was stirred at room temperature for 3 h. The reaction mixture was concentrated in vacuo and the resultant residue was diluted with water (50 mL). The resultant precipitate was collected by filtration and dried in vacuo. The crude material was purified by preparative HPLC (Method A) to afford 6-(4-((4-(2-(2,6-dioxopiperidin-3-yl)-1-oxoisoindolin-4-yl)benzyl)oxy)piperidin-1-yl)-N-(2-((R)-1-methylpyrrolidin-2-yl)-1H-pyrrolo[3,2-c]pyridin-6-yl)nicotinamide (Example 109, 0.18 g, 77%) as an off-white solid. ¹H NMR (400 MHz, DMSO-d₆) δ ppm 1.53-1.60 (m, 2H), 1.79-1.92 (m, 3H), 1.95-2.02 (m, 3H), 2.12-2.14 (m, 1H), 2.16 (s, 3H), 2.24-2.28 (m, 1H), 2.39-2.42 (m, 1H), 2.54-2.61 (m, 2H), 2.86-2.92 (m, 1H), 3.11-3.18 (m, 1H), 3.34-3.39 (m, 2H), 3.72-3.76 (m, 1H), 4.06-4.10 (m, 2H), 4.38-4.44 (m, 1H), 4.59-4.61 (m, 1H), 4.64 (s, 2H), 5.12-5.17 (m, 1H), 6.38 (s, 1H), 6.91 (d, J=8.80 Hz, 1H), 7.49 (d, J=8.0 Hz, 2H), 7.58-7.62 (m, 2H), 7.65 (d, J=7.60 Hz, 1H), 7.71-7.78 (m, 2H), 8.14-8.17 (m, 2H), 8.48 (s, 1H), 8.80 (s, 1H). 10.25 (s, 1H), 10.95 (s, 1H), 11.35 (s, 1H). Mass spec m/z 753.0 [M+H]⁺.

### Example 110 was Prepared Following Scheme 108

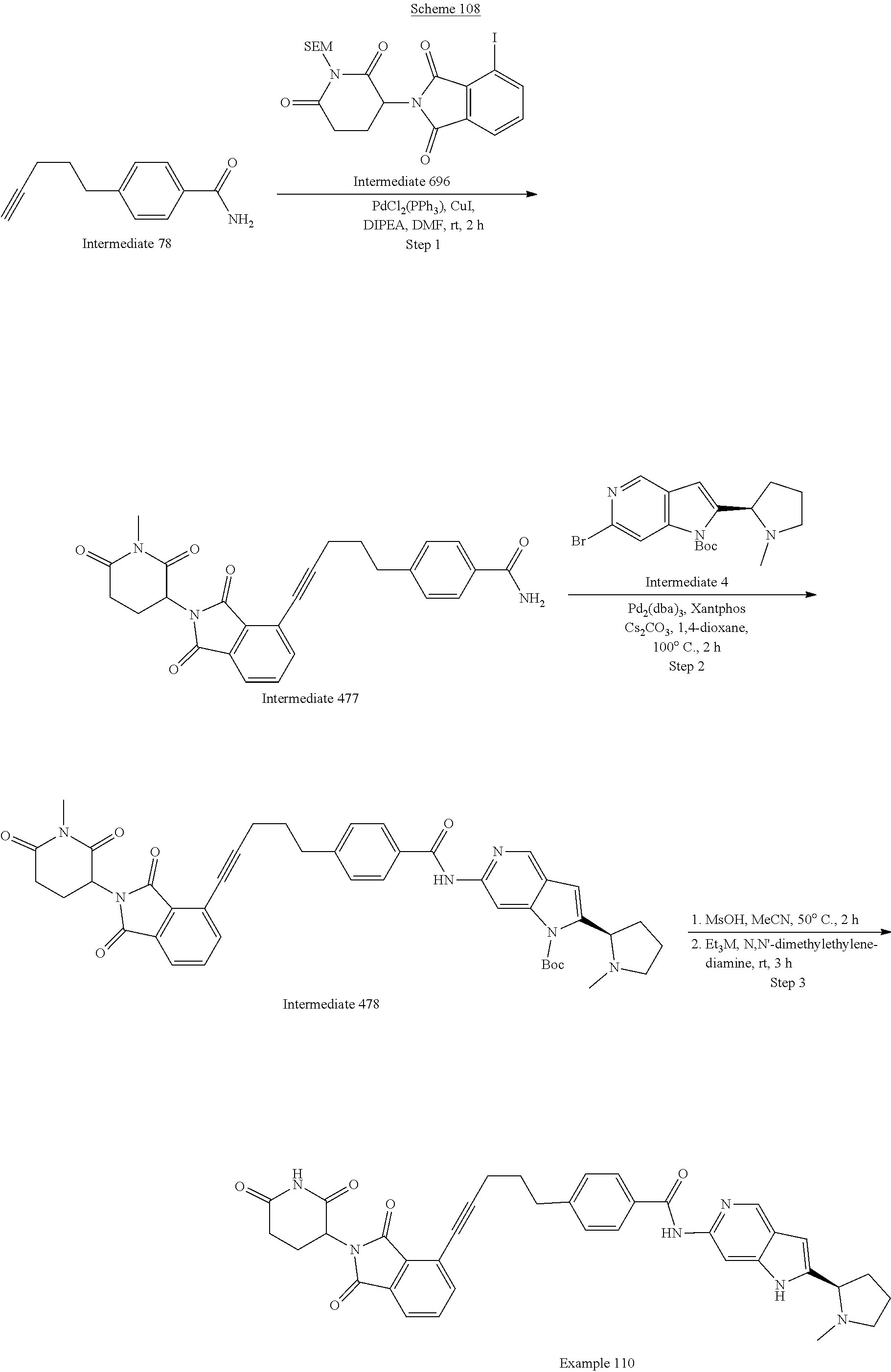 [SMILES: C[Si](C)(C)CCOCN1C(=O)CCC(N2C(=O)c3cccc(I)c3C2=O)C1=O ; C#CCCCc1ccc(C(N)=O)cc1 ; CN1C(=O)CCC(N2C(=O)c3cccc(C#CCCCc4ccc(C(N)=O)cc4)c3C2=O)C1=O ; CN1CCC[C@@H]1c1cc2cnc(Br)cc2n1C(=O)OC(C)(C)C ; CN1C(=O)CCC(N2C(=O)c3cccc(C#CCCCc4ccc(C(=O)Nc5cc6c(cn5)cc([C@H]5CCCN5C)n6C(=O)OC(C)(C)C)cc4)c3C2=O)C1=O ; CN1CCC[C@@H]1c1cc2cnc(NC(=O)c3ccc(CCCC#Cc4cccc5c4C(=O)N(C4CCC(=O)NC4=O)C5=O)cc3)cc2[nH]1]

### Step 1

### Intermediate 477: 4-(5-(2-(2,6-dioxo-1-((2-(trimethylsilyl)ethoxy)methyl)piperidin-3-yl)-1,3-dioxoisoindolin-4-yl)pent-4-yn-1-yl)benzamide

To a suspension of 4-(pent-4-yn-1-yl)benzamide (Intermediate 78, 0.30 g, 1.63 mmol) and 2-(2,6-dioxo-1-((2-(trimethylsilyl)ethoxy)ethyl)piperidin-3-yl)-4-iodoisoindoline-1,3-dione (Intermediate 696, 0.70 g, 1.36 mmol) in dimethylformamide (8 mL) was added N,N-diisopropylethylamine (1.19 mL, 6.80 mmol). The reaction mixture was purged with argon for 15 min, then PdCl₂(PPh₃) (0.10 g, 0.13 mmol) and copper(I) iodide (0.02 g, 0.13 mmol) were added. The reaction mixture was further purged with argon for 10 min and stirred at room temperature for 2 h. The reaction mixture was diluted with water (200 mL) and extracted with ethyl acetate (3×100 mL). The combined organic layers were washed with water (150 mL), dried over anhydrous Na₂SO₄, filtered and concentrated in vacuo. The crude material was purified by combi-flash chromatography, by eluting with 40% ethyl acetate in heptane, to afford 4-(5-(2-(2,6-dioxo-1-((2-(trimethylsilyl)ethoxy)methyl)piperidin-3-yl)-1,3-dioxoisoindolin-4-yl)pent-4-yn-1-yl)benzamide (Intermediate 477, 0.58 g, 74%) as a yellow solid. ¹H NMR (400 MHz, DMSO-d₆) δ ppm −0.04 (s, 9H), 0.78-0.83 (m, 2H), 1.88-1.93 (m, 2H), 2.08-2.11 (m, 1H), 2.55-2.60 (m, 2H), 2.82-2.89 (m, 3H), 2.98-3.07 (m, 1H), 3.22-3.25 (m, 1H), 3.49-3.55 (m, 2H), 5.07 (s, 2H), 5.25-5.30 (m, 1H), 7.25 (br s, 1H), 7.33 (d, J=7.93 Hz, 2H), 7.80 (d, J=7.93 Hz, 2H), 7.84 (br s, 1H), 7.85-7.89 (m, 3H). Mass spec m/z 572.1 [M−H]⁻. Step 2

### Intermediate 478: tert-butyl 6-(4-(5-(2-(2,6-dioxo-1-((2-(trimethylsilyl) ethoxy)methyl)piperidin-3-yl)-1,3-dioxoisoindolin-4-yl)pent-4-yn-1-yl)benzamido)-2-((R)-1-methylpyrrolidin-2-yl)-1H-pyrrolo[3,2-c]pyridine-1-carboxylate

To a solution of 4-(5-(2-(2,6-dioxo-1-((2-(trimethylsilyl)ethoxy)methyl)piperidin-3-yl)-1,3-dioxoisoindolin-4-yl)pent-4-yn-1-yl)benzamide (Intermediate 477, 0.50 g, 0.87 mmol) in 1,4-dioxane (5 mL) was added tert-butyl (R)-6-bromo-2-(1-methylpyrrolidin-2-yl)-1H-pyrrolo[3,2-c]pyridine-1-carboxylate (Intermediate 4, 0.39 g, 1.04 mmol) followed by cesium carbonate (0.85 g, 2.61 mmol). The reaction mixture was purged with argon for 15 min, then Pd₂(dba)₃ (0.12 g, 0.13 mmol) and Xantphos (0.15 g, 0.26 mmol) were added. The reaction mixture was further purged with argon for 10 min and heated at 100° C. for 2 h, then concentrated in vacuo. The crude material was purified by combi-flash chromatography, by eluting with 80% ethyl acetate in heptane, to afford tert-butyl 6-(4-(5-(2-(2,6-dioxo-1-((2-(trimethylsilyl)ethoxy)methyl)piperidin-3-yl)-1,3-dioxoisoindolin-4-yl)pent-4-yn-1-yl)benzamido)-2-((R)-1-methylpyrrolidin-2-yl)-1H-pyrrolo[3,2-c]pyridine-1-carboxylate (Intermediate 478, 0.45 g, 59%) as a pale-yellow solid. ¹H NMR (400 MHz, DMSO-d₆) δ ppm −0.05 (s, 9H), 0.81-0.95 (m, 2H), 1.69 (s, 9H), 1.73-1.78 (m, 3H), 1.88-1.97 (m, 2H), 2.06-2.15 (m, 1H), 2.32 (s, 3H), 2.33-2.37 (m, 2H), 2.55-2.61 (m, 3H), 2.78-2.80 (m, 1H), 2.87-2.95 (m, 2H), 2.98-3.06 (m, 1H), 3.10-3.15 (m, 1H), 3.44-3.58 (m, 2H), 3.87-3.96 (m, 1H), 5.08 (s, 2H), 5.26-5.31 (m, 1H), 6.75 (s, 1H), 7.40 (d, J=7.93 Hz, 2H), 7.83-7.89 (m, 3H), 7.99 (d, J=7.93 Hz, 2H), 8.58 (s, 1H), 8.92 (s, 1H), 10.59 (br s, 1H). Mass spec m/z 873.0 [M+H]⁺.

### Step 3

### Example 110: 4-(5-(2-(2,6-dioxopiperidin-3-yl)-1,3-dioxoisoindolin-4-yl)pent-4-yn-1-yl)-N-(2-((R)-1-methylpyrrolidin-2-yl)-1H-pyrrolo[3,2-c]pyridin-6-yl)benzamide

To a cooled (0° C.) solution of tert-butyl 6-(4-(5-(2-(2,6-dioxo-1-((2-(trimethylsilyl)ethoxy)methyl) piperidin-3-yl)-1,3-dioxoisoindolin-4-yl)pent-4-yn-1-yl)benzamido)-2-((R)-1-methylpyrrolidin-2-yl)-1H-pyrrolo[3,2-c]pyridine-1-carboxylate (Intermediate 478, 0.30 g, 0.34 mmol) in acetonitrile (5 mL) was added trifluoroacetic acid (5 mL, 64.8 mmol) and the reaction mixture was heated at 50° C. for 8 h. The reaction mixture was cooled to 0° C. and diluted with acetonitrile (10 mL). 3M Aqueous sodium bicarbonate solution (10 mL) was added and the mixture stirred at room temperature for 30 min. The reaction mixture was concentrated in vacuo to half volume and the residue was diluted with ice cold water (20 mL). The resultant precipitate was collected by filtration and dried in vacuo. The crude material was purified by preparative HPLC (Method A) to afford 4-(5-(2-(2,6-dioxopiperidin-3-yl)-1,3-dioxoisoindolin-4-yl)pent-4-yn-1-yl)-N-(2-((R)-1-methylpyrrolidin-2-yl)-1H-pyrrolo[3,2-c]pyridin-6-yl)benzamide (Example 110, 0.05 g, 14%) as an off-white solid. ¹H NMR (400 MHz, DMSO-d₆) δ ppm 1.81-1.95 (m, 5H), 2.12-2.16 (m, 2H), 2.17 (s, 3H), 2.28-2.32 (m, 1H), 2.53-2.64 (m, 5H), 2.87-2.96 (m, 3H), 3.14 (t, J=8.25 Hz, 1H), 5.13-5.18 (m, 1H), 6.38 (s, 1H), 7.40 (d, J=8.25 Hz, 2H), 7.83-7.89 (m, 3H), 7.99 (d, J=8.25 Hz, 2H), 8.18 (s, 1H), 8.49 (s, 1H), 10.35 (br s, 1H), 11.14 (br s, 1H), 11.36 (br s, 1H). Mass spec m/z 643.1 [M+H]⁺.

### Example 111 was Synthesised Following Scheme 109

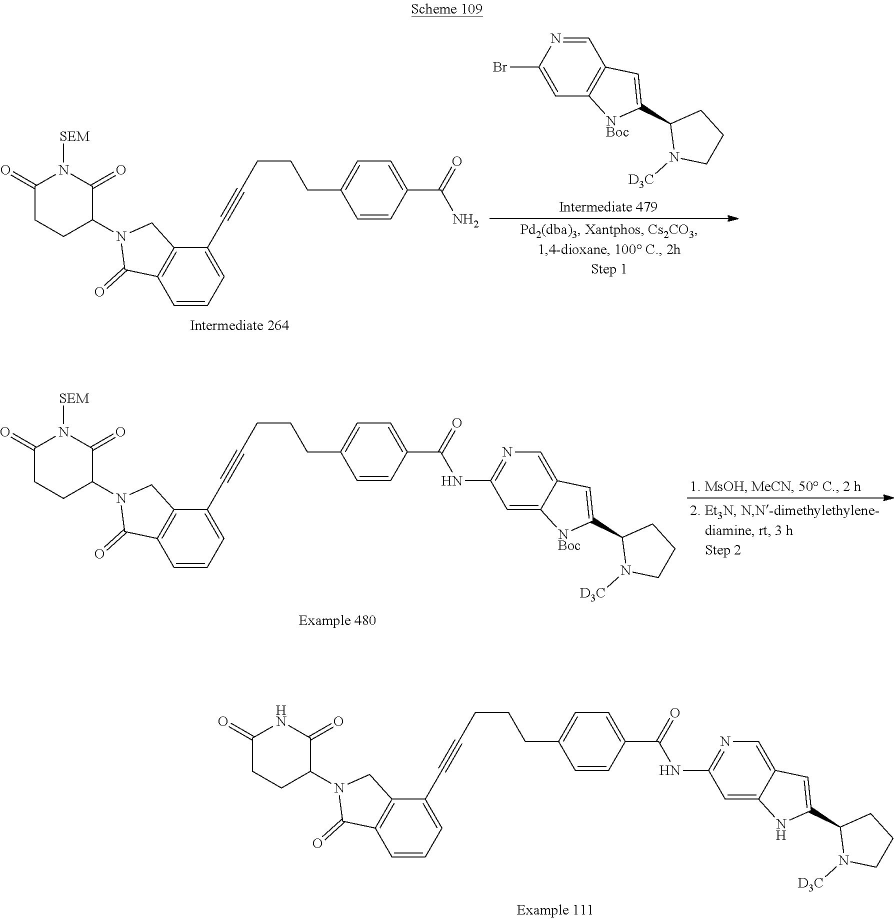 [SMILES: C[Si](C)(C)CCOCN1C(=O)CCC(N2Cc3c(C#CCCCc4ccc(C(N)=O)cc4)cccc3C2=O)C1=O ; [2H]C([2H])([2H])N1CCC[C@@H]1c1cc2cnc(Br)cc2n1C(=O)OC(C)(C)C ; [2H]C([2H])([2H])N1CCC[C@@H]1c1cc2cnc(NC(=O)c3ccc(CCCC#Cc4cccc5c4CN(C4CCC(=O)N(COCC[Si](C)(C)C)C4=O)C5=O)cc3)cc2n1C(=O)OC(C)(C)C ; [2H]C([2H])([2H])N1CCC[C@@H]1c1cc2cnc(NC(=O)c3ccc(CCCC#Cc4cccc5c4CN(C4CCC(=O)NC4=O)C5=O)cc3)cc2[nH]1]

### Step 1

### Intermediate 480: tert-butyl 6-(4-(5-(2-(2,6-dioxo-1-((2-(trimethylsilyl)ethoxy)methyl)piperidin-3-yl)-1-oxoisoindolin-4-yl)pent-4-yn-1-yl)benzamido)-2-((R)-1-(methyl-d₃)pyrrolidin-2-yl)-1H-pyrrolo[3,2-c]pyridine-1-carboxylate

To a solution of 4-(5-(2-(2,6-dioxo-1-((2-(trimethylsilyl)ethoxy)methyl) piperidin-3-yl)-1-oxoisoindolin-4-yl)pent-4-yn-1-yl)benzamide (Intermediate 264, 0.35 g, 0.80 mmol) in 1,4-dioxane (10 mL) was added tert-butyl (R)-6-bromo-2-(1-(methyl-d₃)pyrrolidin-2-yl)-1H-pyrrolo[3,2-c]pyridine-1-carboxylate (Intermediate 479, 0.30 g, 0.78 mmol) followed by cesium carbonate (0.76 g, 2.34 mmol). The reaction mixture was purged with argon for 15 min followed by Pd₂(dba)₃ (0.07 g, 0.07 mmol) and Xantphos (0.13 g, 0.23 mmol) were added. The reaction mixture was further purged with argon for 10 min and then heated at 100° C. for 2 h. After completion, the reaction mixture was concentrated in vacuo to obtain crude compound. The crude material was purified by combi-flash chromatography, by eluting with 90% ethyl acetate in heptane, to afford tert-butyl 6-(4-(5-(2-(2,6-dioxo-1-((2-(trimethylsilyl)ethoxy)methyl)piperidin-3-yl)-1-oxoisoindolin-4-yl)pent-4-yn-1-yl)benzamido)-2-((R)-1-(methyl-d3)pyrrolidin-2-yl)-1H-pyrrolo[3,2-c]pyridine-1-carboxylate (Intermediate 480, 0.20 g, 30%) as an off-white solid. ¹H NMR (400 MHz, DMSO-d₆) δ ppm 0.01 (s, 9H), 0.84-0.89 (m, 2H), 1.23 (t, J=7.09 Hz, 1H), 1.62-1.70 (m, 2H), 1.74 (s, 9H), 1.77-1.82 (m, 2H), 1.94-2.01 (m, 2H), 2.08-2.16 (m, 1H), 2.38-2.48 (m, 3H), 2.86-2.90 (m, 2H), 3.14-3.21 (m, 2H), 3.52-3.60 (m, 2H), 3.94-4.00 (m, 1H), 4.05-4.08 (m, 1H), 4.32-4.36 (m, 1H), 4.53-4.58 (m, 1H), 5.05-5.13 (m, 2H), 5.31-5.35 (m, 1H), 6.80 (s, 1H), 7.43 (d, J=8.0 Hz, 2H), 7.56-7.61 (m, 1H), 7.73-7.80 (m, 2H), 8.05 (d, J=8.0 Hz, 2H), 8.64 (d, J=0.73 Hz, 1H), 8.98 (s, 1H), 10.68 (br s, 1H). Mass spec m/z 862.1 [M+H]⁺.

### Example 111: 4-(5-(2-(2,6-dioxopiperidin-3-yl)-1-oxoisoindolin-4-yl)pent-4-yn-1-yl)-N-(2-((R)-1-(methyl-d₃)pyrrolidin-2-yl)-1H-pyrrolo[3,2-c]pyridin-6-yl)benzamide

To a solution of tert-butyl 6-(4-(5-(2-(2,6-dioxo-1-((2-(trimethylsilyl)ethoxy)methyl)piperidin-3-yl)-1-oxoisoindolin-4-yl)pent-4-yn-1-yl)benzamido)-2-((R)-1-(methyl-d₃)pyrrolidin-2-yl)-1H-pyrrolo[3,2-c]pyridine-1-carboxylate (Intermediate 480, 0.20 g, 0.23 mmol) in acetonitrile (4 mL) was added methanesulfonic acid (0.15 mL, 2.32 mmol) and the reaction mixture was heated at 50° C. for 2 h. After cooling to room temperature, N,N′-dimethylethylenediamine (0.16 mL, 1.39 mmol) followed by triethylamine (0.47 g, 4.64 mmol) were added, and the reaction mixture was stirred at room temperature for 3 h. The reaction mixture was then diluted with water (100 mL), the resultant precipitate was collected by filtration and dried in vacuo. The crude material was purified by preparative HPLC (Method A) to afford 4-(5-(2-(2,6-dioxopiperidin-3-yl)-1-oxoisoindolin-4-yl)pent-4-yn-1-yl)-N-(2-((R)-1-(methyl-d3)pyrrolidin-2-yl)-1H-pyrrolo[3,2-c]pyridin-6-yl)benzamide (Example 111, 0.07 g, 51%) as an off-white solid. ¹H NMR (400 MHz, DMSO-d₆) δ ppm 1.78-1.96 (m, 5H), 1.98-2.05 (m, 1H), 2.11-2.19 (m, 1H), 2.22-2.29 (m, 1H), 2.52-2.63 (m, 4H), 2.83 (t, J=7.58 Hz, 2H), 2.87-2.97 (m, 1H), 3.10-3.17 (m, 1H), 3.28-3.32 (m, 1H), 4.33-4.39 (m, 1H), 4.46-4.53 (m, 1H), 5.12-5.17 (m, 1H), 6.39 (s, 1H), 7.38 (d, J=8.31 Hz, 2H), 7.50-7.56 (m, 1H), 7.68 (dd, J=7.60, 0.92 Hz, 1H), 7.72 (dd, J=7.60, 0.73 Hz, 1H), 8.00 (d, J=8.0 Hz, 2H), 8.19 (s, 1H), 8.50 (s, 1H), 10.39 (br s, 1H), 11.05 (br s, 1H), 11.39 (br s, 1H). Mass spec m/z 632.0 [M+H]⁺.

### Intermediate 479 was Synthesised Following Scheme 110

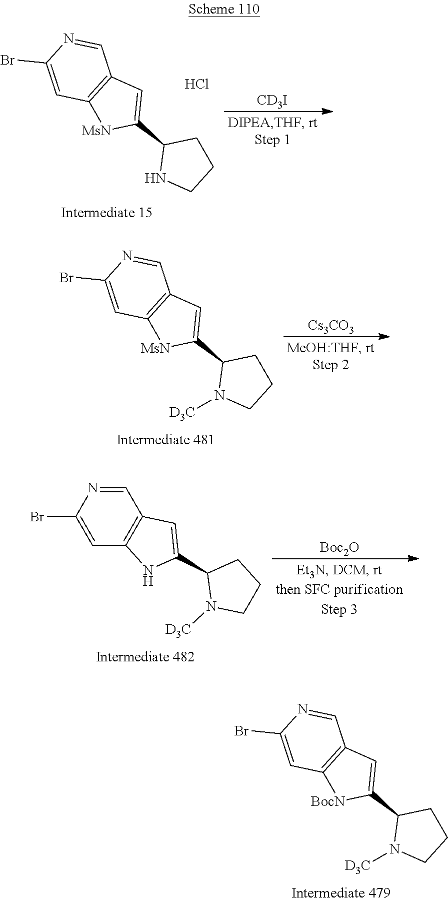

### Step 1

### Intermediate 481: (R)-6-bromo-2-(1-(methyl-d3) pyrrolidin-2-yl)-1-(methylsulfonyl)-1H-pyrrolo[3,2-c]pyridine

To a cooled (0° C.) solution of (R)-6-bromo-1-(methylsulfonyl)-2-(pyrrolidin-2-yl)-1H-pyrrolo[3,2-c]pyridine hydrochloride (Intermediate 15, 10.0 g, 29.1 mmol) in tetrahydrofuran (40 mL) was added N,N-diisopropylethylamine (15.8 g, 116 mmol) followed by iodomethane-d₃ (6.32 g, 43.6 mmol) and the reaction mixture was stirred at room temperature for 16 h. The reaction mixture was then poured into water (100 mL) and extracted with ethyl acetate (3×150 mL). The combined organic layers were dried over anhydrous Na₂SO₄, filtered, and concentrated in vacuo. The crude material was purified by combi-flash chromatography, by eluting with 80% ethyl acetate in heptane, to afford (R)-6-bromo-2-(1-(methyl-d3)pyrrolidin-2-yl)-1-(methylsulfonyl)-1H-pyrrolo[3,2-c]pyridine (Intermediate 481, 5.2 g, 50%) as a yellow solid. ¹H NMR (400 MHz, DMSO-d₆) δ ppm 1.68-1.80 (m, 3H), 2.29-2.38 (m, 2H), 3.10-3.12 (m, 1H), 3.56 (s, 3H), 3.77-3.81 (m, 1H), 6.89 (s, 1H), 7.96 (s, 1H), 8.68 (s, 1H). Mass spec m/z 362.8 [M+H+2]⁺.

### Step 2

### Intermediate 482: (R)-6-bromo-2-(1-(methyl-d3)pyrrolidin-2-yl)-1H-pyrrolo[3,2-c]pyridine

To a cooled (0° C.) solution of (R)-6-bromo-2-(1-(methyl-d3) pyrrolidin-2-yl)-1-(methylsulfonyl)-1H-pyrrolo[3,2-c]pyridine (Intermediate 481, 5.0 g, 14 mmol) in tetrahydrofuran (20 mL) and methanol (20 mL) was added cesium carbonate (9.0 g, 28 mmol), then the reaction mixture was stirred at room temperature for 2 h. The reaction mixture was poured into ice cold water (100 mL) and extracted with dichloromethane (3×100 mL). The combined organic layers were dried over anhydrous Na₂SO₄, filtered, and concentrated in vacuo to afford (R)-6-bromo-2-(1-(methyl-d3) pyrrolidin-2-yl)-1H-pyrrolo[3,2-c]pyridine (Intermediate 482, 3.2 g, 77%) as an off-white solid which was used for the next step without further purification. ¹H NMR (400 MHz, DMSO-d₆) δ ppm 1.70-1.94 (m, 3H), 2.12-2.19 (m, 1H), 2.23-2.30 (m, 1H), 3.13-3.15 (m, 1H), 3.32-3.38 (m, 1H), 6.45 (s, 1H), 7.43 (s, 1H), 8.48 (s, 1H), 11.57 (br s, 1H).

### Step 3

### Intermediate 479: tert-butyl (R)-6-bromo-2-(1-(methyl-d3) pyrrolidin-2-yl)-1H-pyrrolo[3,2-c]pyridine-1-carboxylate

To a solution of (R)-6-bromo-2-(1-(methyl-d3) pyrrolidin-2-yl)-1H-pyrrolo[3,2-c]pyridine (Intermediate 482, 3.0 g, 11 mmol) in dichloromethane (30 mL) was added triethylamine (3.2 g, 32 mmol) followed by di-tert-butyl dicarbonate (3.5 g, 16 mmol) and 4-dimethylaminopyridine (0.13 g, 1.1 mmol) and the reaction mixture was stirred at room temperature for 16 h. The reaction mixture was poured into water (100 mL) and extracted with dichloromethane (3×100 mL). The combined organic layers were dried over anhydrous Na₂SO₄, filtered, and concentrated in vacuo to obtain the crude compound. The crude material was purified by combi-flash chromatography, by eluting with 50% ethyl acetate in heptane, to afford tert-butyl (R)-6-bromo-2-(1-(methyl-d3)pyrrolidin-2-yl)-1H-pyrrolo[3,2-c]pyridine-1-carboxylate (2.5 g) as an off-white solid. This material was further purified by SFC chiral purification (Column: Chiralpak IK (250*30 mm) 5 um. Isocratic elution with 25% Mobile Phase (A): CO₂; mobile Phase (B): MeCN+isopropylalcohol) to afford (R)-6-bromo-2-(1-(methyl-d3) pyrrolidin-2-yl)-1H-pyrrolo[3,2-c]pyridine-1-carboxylate (Intermediate 479, 1.8 g, 44%) as an off-white solid. ¹H NMR (400 MHz, DMSO-d₆) δ ppm 1.65 (s, 9H), 1.69-1.81 (m, 3H), 2.31-2.36 (m, 2H), 3.07-3.14 (m, 1H), 3.85-3.62 (m, 1H), 6.79 (s, 1H), 8.04 (s, 1H), 8.60 (s, 1H). Mass spec m/z 384.9 [M+H+2]⁺.

### Example 112 was Prepared Following Scheme 111

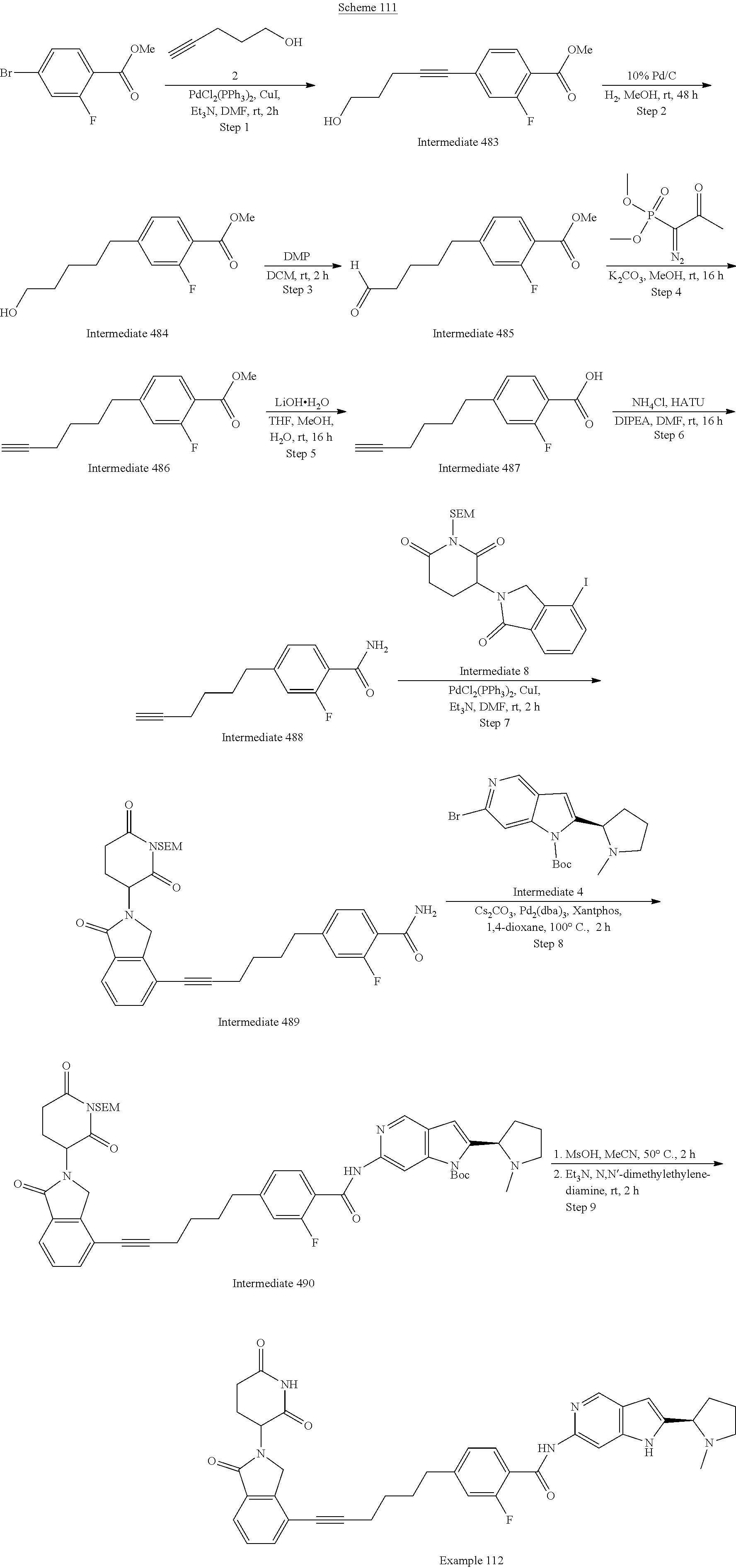

### Step 1

### Intermediate 483: Methyl 2-fluoro-4-(5-hydroxypent-1-yn-1-yl)benzoate

To a solution of methyl 4-bromo-2-fluorobenzoate (CAS No. 179232-29-2, 10.0 g, 42.91 mmol) in dimethylformamide (25 mL) was added pent-4-yn-1-ol (CAS No: 5390-04-5, 5.99 mL, 64.37 mmol) followed by triethylamine (210 mL, 1502.0 mmol). The reaction mixture was purged with argon for 15 min, then copper (I) iodide (0.86 g, 4.293 mmol) and PdCl₂(PPh₃)₂ (3.11 g, 4.29 mmol) were added. The reaction mixture was further purged with argon for 10 mins and stirred at room temperature for 2 h. After completion, the reaction mixture was poured into ice cold water (200 mL) and extracted with ethyl acetate (3×100 mL). The combined organic layers were washed with brine solution (100 mL), dried over anhydrous Na₂SO₄, and concentrated in vacuo. The crude material was purified by combi-flash chromatography, by eluting with 20% ethyl acetate in heptane, to afford methyl 2-fluoro-4-(5-hydroxypent-1-yn-1-yl)benzoate (Intermediate 483, 9.00 g, 89%) as a yellow solid. ¹H NMR (400 MHz, DMSO-d₆) δ ppm 1.65-1.74 (m, 2H), 3.48-3.56 (m, 2H), 3.85 (s, 3H), 4.54-4.58 (m, 1H), 7.30-7.40 (m, 2H), 7.84 (t, J=7.88 Hz, 1H). Mass spec m/z 237.0 [M+H]⁺.

### Step 2

### Intermediate 484: Methyl 2-fluoro-4-(5-hydroxypentyl)benzoate

To a solution of methyl 2-fluoro-4-(5-hydroxypent-1-yn-1-yl)benzoate (Intermediate 483, 8.00 g, 33.9 mmol) in methanol (100 mL) was added 10% Pd/C (3.50 g) and the reaction mixture was stirred at room temperature for 48 h under H₂ atmosphere at 140 psi. After completion, the reaction mixture was filtered through a celite bed, the filter pad was washed with MeOH (250 mL), and the filtrate concentrated in vacuo to afford methyl 2-fluoro-4-(5-hydroxypentyl)benzoate (Intermediate 484, 7.00 g, 86%) as a colourless liquid, which was used for the next step without further purification. ¹H NMR (400 MHz, DMSO-d₆) δ ppm 1.22-1.34 (m, 2H), 1.38-1.47 (m, 2H), 1.53-1.63 (m, 2H), 2.62-2.66 (m, 2H), 3.34-3.40 (m, 2H), 3.83 (s, 3H), 4.32-4.36 (m, 1H), 7.13-7.22 (m, 2H), 7.79 (t, J=7.88 Hz, 1H). Mass spec m/z 241.2 [M+H]⁺.

### Step 3

### Intermediate 485: Methyl 2-fluoro-4-(5-oxopentyl)benzoate

To a cooled (0° C.) solution of methyl 2-fluoro-4-(5-hydroxypentyl)benzoate (Intermediate 484, 7.00 g, 29 mmol) in dichloromethane (100 mL) was added Dess-Martin periodinane (19.0 g, 44 mmol) and the reaction mixture was stirred at room temperature for 2 h. After completion, the reaction mixture was diluted with dichloromethane (250 mL) and poured into saturated aqueous sodium bicarbonate solution (250 mL). The organic layer was separated, washed with saturated aqueous sodium thiosulfate solution (250 mL), dried over anhydrous Na₂SO₄, and concentrated in vacuo to afford methyl 2-fluoro-4-(5-oxopentyl)benzoate (Intermediate 485, 6.80 g, 98%) as a colourless oil, which was used for the next step without further purification. ¹H NMR (400 MHz, DMSO-d₆) δ ppm 1.43-1.66 (m, 4H), 2.40-2.46 (m, 2H), 2.60-2.68 (m, 2H), 3.83 (s, 3H), 7.12-7.24 (m, 2H), 7.76-7.89 (m, 1H), 9.65 (s, 1H). Mass spec m/z 239.0 [M+H]⁺.

### Step 4

### Intermediate 486: Methyl 2-fluoro-4-(hex-5-yn-1-yl)benzoate

To a cooled (0° C.) solution of methyl 2-fluoro-4-(5-oxopentyl)benzoate (Intermediate 485, 6.80 g, 29 mmol) in methanol (100 mL) was added potassium carbonate (5.90 g, 43 mmol), followed by dimethyl (1-diazo-2-oxopropyl)phosphonate (CAS No. 90965-06-3, 7.1 mL, 43 mmol) dropwise over 5 min. The reaction mixture was stirred at room temperature for 16 h, then concentrated in vacuo. The resulting residue was diluted with ice cold water (200 mL) and extracted with dichloromethane (2×200 mL). The combined organic layers were separated, dried over anhydrous Na₂SO₄, and concentrated in vacuo. The crude material was purified by combi-flash chromatography, by eluting with 10% ethyl acetate in heptane, to afford methyl 2-fluoro-4-(hex-5-yn-1-yl)benzoate (Intermediate 486, 2.30 g, 34%) as a colourless liquid. ¹H NMR (400 MHz, DMSO-d₆) δ ppm 1.40-1.48 (m, 2H), 1.62-1.72 (m, 2H), 2.14-2.22 (m, 2H), 2.66 (t, J=7.46 Hz, 2H), 2.75 (s, 1H), 3.83 (s, 3H), 7.14-7.23 (m, 2H), 7.81 (t, J=7.88 Hz, 1H). Mass spec m/z 235.1 [M+H]⁺.

### Step 5

### Intermediate 487: 2-fluoro-4-(hex-5-yn-1-yl)benzoic acid

To a solution of methyl 2-fluoro-4-(hex-5-yn-1-yl)benzoate (Intermediate 486, 2.30 g, 9.8 mmol) in tetrahydrofuran (40 mL), methanol (10 mL) and water (10 mL) was added lithium hydroxide (0.72 g, 29 mmol). The reaction mixture was stirred at room temperature for 16 h. After completion, the reaction mixture was concentrated in vacuo. The resulting residue was acidified to pH~5 with 1N aqueous HCl solution, resultant precipitate was collected by filtration, washed with water (50 mL) and heptane (50 mL) then dried in vacuo to afford 2-fluoro-4-(hex-5-yn-1-yl)benzoic acid (Intermediate 487, 1.80 g, 83%) as an off-white solid, which was used for the next step without further purification. ¹H NMR (400 MHz, DMSO-d₆) δ ppm 1.40-1.48 (m, 2H), 1.62-1.70 (m, 2H), 2.14-2.20 (m, 2H), 2.65 (t, J=7.46 Hz, 2H), 2.74 (s, 1H), 7.10-7.17 (m, 2H), 7.77 (t, J=7.88 Hz, 1H). Mass spec m/z 221.15 [M+H]⁺.

### Step 6

### Intermediate 488: 2-fluoro-4-(hex-5-yn-1-yl)benzamide

To a solution of 2-fluoro-4-(hex-5-yn-1-yl)benzoic acid (Intermediate 487, 1.80 g, 8.2 mmol) in dimethylformamide (20 mL) were added HATU (6.40 g, 16 mmol) and N,N-diisopropylethylamine (4.3 mL, 25 mmol). Ammonium chloride (2.2 g, 41 mmol) was then added and the reaction mixture was stirred at room temperature for 16 h. After completion, the reaction mixture was poured into ice cold water (100 mL), the resultant precipitate was collected by filtration, washed with water (2×50 mL) and dried in vacuo to afford 2-fluoro-4-(hex-5-yn-1-yl)benzamide (Intermediate 488, 1.50 g, 84%) as an off-white solid, which was used for the next step without further purification. ¹H NMR (400 MHz, DMSO-d₆) δ ppm 1.40-1.48 (m, 2H), 1.60-1.72 (m, 2H), 2.15-2.22 (m, 2H), 2.63 (t, J=7.46 Hz, 2H), 2.76 (s, 1H), 7.06-7.15 (m, 2H), 7.52-7.62 (m, 3H). Mass spec m/z 220.1 [M+H]⁺.

### Step 7

### Intermediate 489: 4-(6-(2-(2,6-dioxo-1-((2-(trimethylsilyl)ethoxy)methyl)piperidin-3-yl)-1-oxoisoindolin-4-yl)hex-5-yn-1-yl)-2-fluorobenzamide

To a solution of 2-fluoro-4-(hex-5-yn-1-yl)benzamide (Intermediate 488, 0.60 g, 2.74 mmol) in dimethylformamide (4 mL) was added 3-(4-iodo-1-oxoisoindolin-2-yl)-1-((2-(trimethylsilyl)ethoxy)methyl)piperidine-2,6-dione (Intermediate 8, 1.37 g, 2.74 mmol) followed by triethylamine (13.4 mL, 95.80 mmol). The reaction mixture was purged with argon for 15 min then copper (I) iodide (0.055 g, 0.27 mmol) and PdCl₂(PPh₃)₂ (0.19 g, 0.27 mmol) were added. The reaction mixture was further purged with argon for 10 min and stirred at room temperature for 2 h. After completion, the reaction mixture was poured into ice cold water (150 mL) and extracted with ethyl acetate (2×150 mL). The combined organic layers were washed with brine solution (100 mL), dried over anhydrous Na₂SO₄, and concentrated in vacuo. The crude material was purified by combi-flash chromatography, by eluting with 65% ethyl acetate in heptane, to afford 4-(6-(2-(2,6-dioxo-1-((2-(trimethylsilyl)ethoxy)methyl)piperidin-3-yl)-1-oxoisoindolin-4-yl)hex-5-yn-1-yl)-2-fluorobenzamide (Intermediate 489, 1.01 g, 62%) as a yellow solid. ¹H NMR (400 MHz, DMSO-d₆) δ ppm −0.04 (s, 9H), 0.76-0.90 (m, 2H), 1.39-1.50 (m, 1H), 1.54-1.66 (m, 2H), 1.68-1.80 (m, 2H), 2.00-2.10 (m, 1H), 2.30-2.43 (m, 2H), 2.66-2.70 (m, 2H), 2.75-2.82 (m, 1H), 2.99-3.13 (m, 1H), 3.46-3.56 (m, 2H), 4.22-4.28 (m, 1H), 4.43-4.48 (m, 1H), 4.99-5.12 (m, 2H), 5.24-5.30 (m, 1H), 7.07-7.16 (m, 2H), 7.50-7.61 (m, 4H), 7.62-7.66 (m, 1H), 7.72 (d, J=7.60 Hz, 1H). Mass spec m/z 590.0 [M+H]⁺.

### Step 8

### Intermediate 490: tert-butyl 6-(4-(6-(2-(2,6-dioxo-1-((2-(trimethylsilyl)ethoxy)methyl)piperidin-3-yl)-1-oxoisoindolin-4-yl)hex-5-yn-1-yl)-2-fluorobenzamido)-2-((R)-1-methylpyrrolidin-2-yl)-1H-pyrrolo[3,2-c]pyridine-1-carboxylate

To a solution of 4-(6-(2-(2,6-dioxo-1-((2-(trimethylsilyl) ethoxy)methyl)piperidin-3-yl)-1-oxoisoindolin-4-yl)hex-5-yn-1-yl)-2-fluorobenzamide (Intermediate 489, 0.60 g, 1.01 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 0.46 g, 1.22 mmol) followed by cesium carbonate (0.99 g, 3.04 mmol). The reaction mixture was purged with argon for 15 min then Pd₂(dba)₃ (0.095 g, 0.10 mmol) and Xantphos (0.060 g, 0.10 mmol) were added. The reaction mixture was further purged with argon for 10 min and heated at 100° C. for 2 h. After completion, the reaction mixture was poured into ice cold water (100 mL) and extracted with ethyl acetate (2×100 mL). The combined organic layers were washed with brine solution (50 mL), dried over anhydrous Na₂SO₄, and concentrated in vacuo. The crude material was purified by combi-flash chromatography, by eluting with 90% ethyl acetate in heptane, to afford tert-butyl 6-(4-(6-(2-(2,6-dioxo-1-((2-(trimethylsilyl)ethoxy)methyl)piperidin-3-yl)-1-oxoisoindolin-4-yl)hex-5-yn-1-yl)-2-fluorobenzamido)-2-((R)-1-methylpyrrolidin-2-yl)-1H-pyrrolo[3,2-c]pyridine-1-carboxylate (Intermediate 490, 0.50 g, 55%) as a yellow solid. ¹H NMR (400 MHz, DMSO-d₆) δ ppm −0.05 (s, 9H), 0.76-0.88 (m, 2H), 1.54-1.65 (m, 4H), 1.69 (s, 9H), 1.73-1.82 (m, 4H), 2.02-2.10 (m, 1H), 2.28-2.32 (m, 1H), 2.35 (s, 3H), 2.38-2.45 (m, 1H), 2.52-2.57 (m, 2H), 2.63-2.79 (m, 3H), 3.00-3.17 (m, 2H), 3.44-3.58 (m, 2H), 3.90-3.96 (m, 1H), 4.24-4.30 (m, 1H), 4.44-4.50 (m, 1H), 4.96-5.09 (m, 2H), 5.18-5.30 (m, 1H), 6.74 (s, 1H), 7.14-7.23 (m, 2H), 7.49-7.56 (m, 1H), 7.61-7.68 (m, 2H), 7.72 (d, J=7.88 Hz, 1H), 8.55 (s, 1H), 8.92 (s, 1H), 10.48 (br s, 1H). Mass spec m/z 890.6 [M+H]⁺.

### Step 9

### Example 112: 4-(6-(2-(2,6-dioxopiperidin-3-yl)-1-oxoisoindolin-4-yl)hex-5-yn-1-yl)-2-fluoro-N-(2-((R)-1-methylpyrrolidin-2-yl)-1H-pyrrolo[3,2-c]pyridin-6-yl)benzamide

To a solution of tert-butyl 6-(4-(6-(2-(2,6-dioxo-1-((2-(trimethylsilyl)ethoxy)methyl)piperidin-3-yl)-1-oxoisoindolin-4-yl)hex-5-yn-1-yl)-2-fluorobenzamido)-2-((R)-1-methylpyrrolidin-2-yl)-1H-pyrrolo[3,2-c]pyridine-1-carboxylate (Intermediate 490, 0.45 g, 0.50 mmol) in acetonitrile (10 mL) was added methanesulfonic acid (0.33 mL, 5.05 mmol) and the reaction mixture was heated at 50° C. for 2 h. After cooling to room temperature, N,N′-dimethylethylenediamine (0.60 mL, 5.05 mmol) followed by triethylamine (1.41 mL, 10.10 mmol) were added and the reaction mixture was stirred at room temperature for 2 h. After completion, the reaction mixture was poured into ice cold water (50 mL), the resultant precipitate was collected by filtration, washed with heptane (2×40 mL) and then dried in vacuo. The crude material was purified by preparative HPLC (Method A) to afford 4-(6-(2-(2,6-dioxopiperidin-3-yl)-1-oxoisoindolin-4-yl)hex-5-yn-1-yl)-2-fluoro-N-(2-((R)-1-methylpyrrolidin-2-yl)-1H-pyrrolo[3,2-c]pyridin-6-yl)benzamide (Example 112, 0.14 g, 41%) as an off-white solid. ¹H NMR (400 MHz, DMSO-d₆) δ ppm 1.78-1.83 (m, 2H), 1.84-2.02 (m, 5H), 2.03-2.08 (m, 1H), 2.12-2.14 (m, 1H), 2.16 (s, 3H), 2.25-2.34 (m, 1H), 2.42-2.46 (m, 1H), 2.52-2.61 (m, 3H), 2.64-2.68 (m, 2H), 2.79-2.89 (m, 1H), 3.11-3.18 (m, 1H), 4.32-4.34 (m, 1H), 4.29-4.34 (m, 1H), 4.42-4.48 (m, 1H), 5.10-5.16 (m, 1H), 6.39 (s, 1H), 7.16-7.22 (m, 2H), 7.50-7.56 (m, 1H), 7.61-7.72 (m, 3H), 8.19 (s, 1H), 8.46 (s, 1H), 10.16 (d, J=4.40 Hz, 1H), 11.00 (br s, 1H), 11.37 (s, 1H). Mass spec m/z 661.1 [M+H]⁺.

### Example 113 was Prepared Following Scheme 112

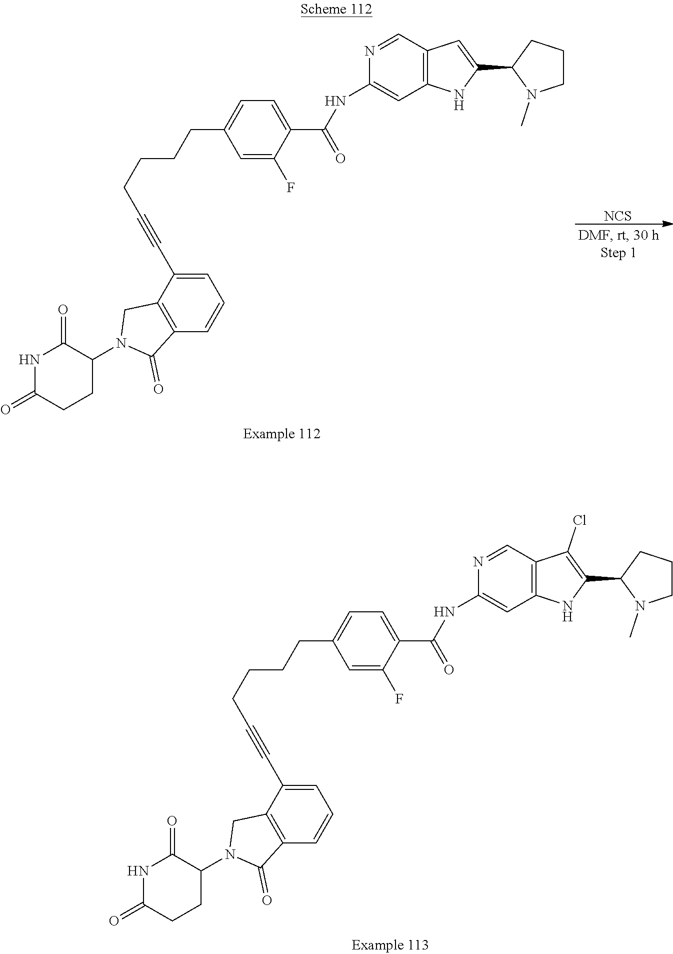 [SMILES: CN1CCC[C@@H]1c1cc2cnc(NC(=O)c3ccc(CCCCC#Cc4cccc5c4CN(C4CCC(=O)NC4=O)C5=O)cc3F)cc2[nH]1 ; "NCS" ; CN1CCC[C@@H]1c1[nH]c2cc(NC(=O)c3ccc(CCCCC#Cc4cccc5c4CN(C4CCC(=O)NC4=O)C5=O)cc3F)ncc2c1Cl]

### Step 1

### Example 113: N-(3-chloro-2-((R)-1-methylpyrrolidin-2-yl)-1H-pyrrolo[3,2-c]pyridin-6-yl)-4-(6-(2-(2,6-dioxopiperidin-3-yl)-1-oxoisoindolin-4-yl)hex-5-yn-1-yl)-2-fluorobenzamide

To a solution of 4-(6-(2-(2,6-dioxopiperidin-3-yl)-1-oxoisoindolin-4-yl)hex-5-yn-1-yl)-2-fluoro-N-(2-((R)-1-methylpyrrolidin-2-yl)-1H-pyrrolo[3,2-c]pyridin-6-yl)benzamide (Example 112, 0.138 g, 0.21 mmol) in dimethylformamide (5.0 mL) was added N-chlorosuccinimide (0.18 g, 0.031 mmol) and the reaction mixture was stirred at room temperature for 30 h. After completion, the reaction mixture was quenched with ice cold water (50 mL) and the resultant precipitate was collected by filtration and dried in vacuo. The crude material was purified by preparative HPLC (Method A) to afford N-(3-chloro-2-((R)-1-methylpyrrolidin-2-yl)-1H-pyrrolo[3,2-c]pyridin-6-yl)-4-(6-(2-(2,6-dioxopiperidin-3-yl)-1-oxoisoindolin-4-yl)hex-5-yn-1-yl)-2-fluorobenzamide (Example 113, 0.05 g, 38%) as an off-white solid. ¹H NMR (400 MHz, DMSO-d₆) δ ppm 1.56-1.66 (m, 2H), 1.73-2.02 (m, 6H), 2.15 (s, 3H), 2.18-2.21 (m, 1H), 2.27-2.33 (m, 1H), 2.45-2.47 (m, 1H), 2.51-2.57 (m, 2H), 2.61-2.64 (m, 1H), 2.73 (t, J=7.60 Hz, 1H), 2.83-2.97 (m, 2H), 3.15-3.18 (m, 1H), 3.49-3.53 (m, 1H), 4.26-4.36 (m, 1H), 4.39-4.49 (m, 1H), 5.11-5.15 (m, 1H), 7.17-7.22 (m, 2H), 7.52 (t, J=7.60 Hz, 1H), 7.63-7.71 (m, 3H), 8.24 (s, 1H), 8.46 (s, 1H), 10.37 (d, J=3.25 Hz, 1H), 10.99 (br s, 1H), 11.69 (br s, 1H). Mass spec m/z 695.0 [M+H]⁺.

### Example 114 was Synthesised Following Scheme 113

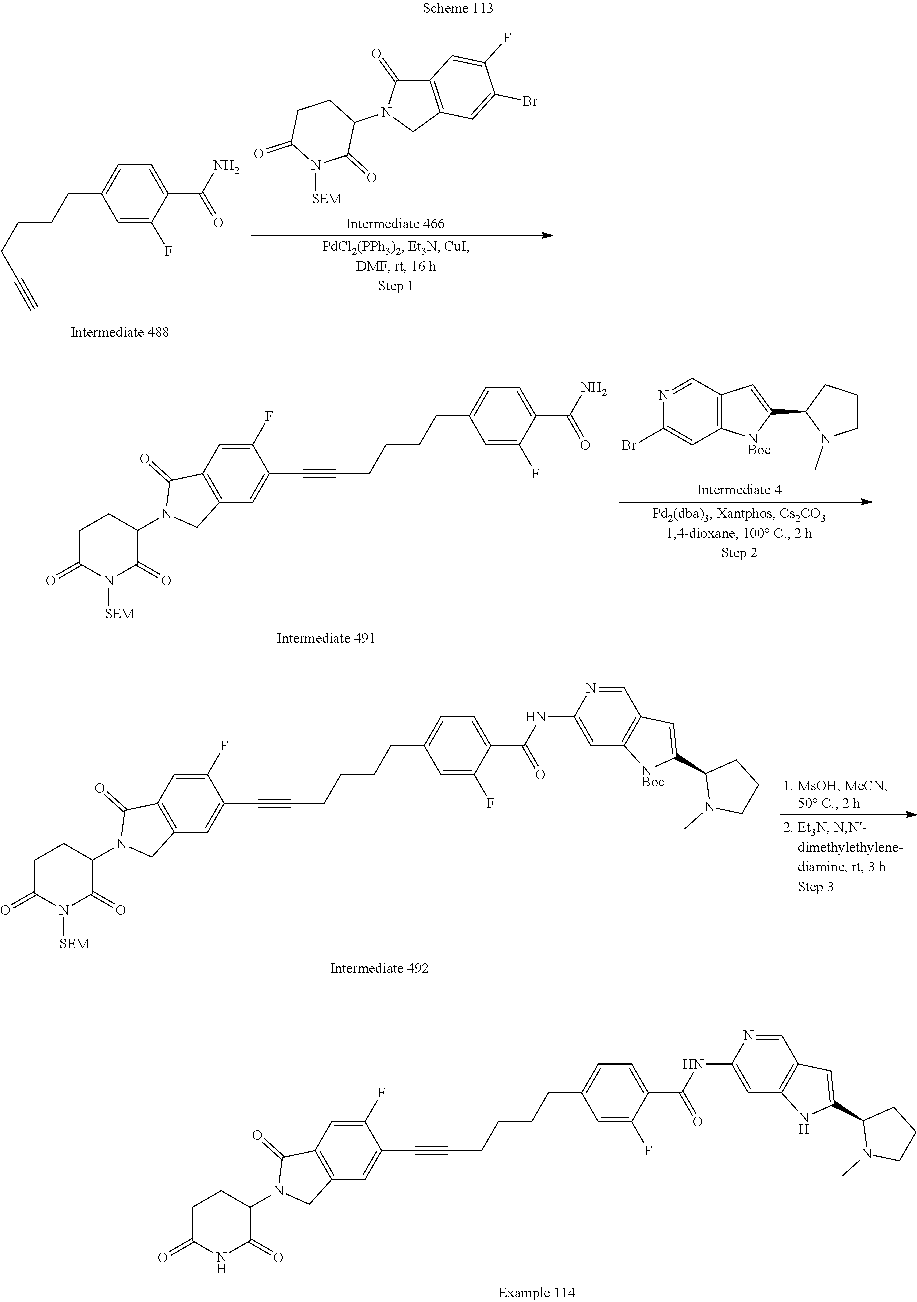

### Step 1

### Intermediate 491: 4-(6-(2-(2,6-dioxo-1-((2-(trimethylsilyl)ethoxy)methyl)piperidin-3-yl)-6-fluoro-1-oxoisoindolin-5-yl)hex-5-yn-1-yl)-2-fluorobenzamide

To a solution of 2-fluoro-4-(hex-5-yn-1-yl)benzamide (Intermediate 488, 0.41 g, 1.90 mmol) and 3-(5-bromo-6-fluoro-1-oxoisoindolin-2-yl)-1-((2-(trimethylsilyl)ethoxy)methyl)piperidine-2,6-dione (Intermediate 466, 0.3 g, 0.63 mmol) in dimethylformamide (15 mL) was added triethylamine (0.71 mL, 5.09 mmol). The reaction mixture was purged with argon for 15 min then copper (I) iodide (0.01 g, 0.06 mmol) and PdCl₂(PPh₃)₂ (0.04 g, 0.06 mmol) were added. The reaction mixture was purged with argon for a further 10 min and stirred at room temperature for 16 h. After completion, the reaction mixture was concentrated in vacuo and the resulting crude material was purified by combi-flash chromatography, by eluting with 30% ethyl acetate in heptane, to afford 4-(6-(2-(2,6-dioxo-1-((2-(trimethylsilyl)ethoxy)methyl)piperidin-3-yl)-6-fluoro-1-oxoisoindolin-5-yl)hex-5-yn-1-yl)-2-fluorobenzamide (Intermediate 491, 0.35 g, 90%) as a green solid. ¹H NMR (400 MHz, DMSO-d₆) δ ppm −0.04 (s, 9H), 82-0.89 (m, 2H), 1.44-1.51 (m, 1H), 1.56-1.69 (m, 2H), 1.73-1.82 (m, 1H), 2.04-2.12 (m, 1H), 2.31-2.37 (m, 1H), 2.55-2.61 (m, 2H), 2.62-2.74 (m, 3H), 3.05-3.15 (m, 2H), 3.38-3.42 (m, 2H), 3.50-3.58 (m, 1H), 4.26-4.33 (m, 1H), 4.43-4.49 (m, 1H), 5.03-5.10 (m, 1H), 7.12-7.20 (m, 2H), 7.59-7.63 (m, 3H), 7.76 (d, J=60 Hz, 1H), 7.97 (br s, 1H). Mass spec m/z 608.2 [M−H]⁻.

### Step 2

### Intermediate 492: tert-butyl 6-(4-(6-(2-(2,6-dioxo-1-((2-(trimethylsilyl)ethoxy)methyl)piperidin-3-yl)-6-fluoro-1-oxoisoindolin-5-yl)hex-5-yn-1-yl)-2-fluorobenzamido)-2-((R)-1-methylpyrrolidin-2-yl)-1H-pyrrolo[3,2-c]pyridine-1-carboxylate

To a solution of 4-(6-(2-(2,6-dioxo-1-((2-(trimethylsilyl)ethoxy)methyl)piperidin-3-yl)-6-fluoro-1-oxoisoindolin-5-yl)hex-5-yn-1-yl)-2-fluorobenzamide (Intermediate 491, 0.35 g, 0.57 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.26 g, 0.68 mmol) followed by cesium carbonate (0.46 g, 1.43 mmol). The reaction mixture was purged with argon for 10 min then Pd₂(dba)₃ (0.08 g, 0.08 mmol) and Xantphos (0.10 g, 0.17 mmol) were added. The reaction mixture was purged with argon for another 10 min and stirred at 100° C. for 2 h. After completion, the reaction mixture was filtered through a celite bed, the filter pad was washed with ethyl acetate (200 mL), and the filtrate concentrated in vacuo. The crude material was purified by combi-flash chromatography, by eluting with 90% ethyl acetate in heptane, to afford tert-butyl 6-(4-(6-(2-(2,6-dioxo-1-((2-(trimethylsilyl)ethoxy)methyl)piperidin-3-yl)-6-fluoro-1-oxoisoindolin-5-yl)hex-5-yn-1-yl)-2-fluorobenzamido)-2-((R)-1-methylpyrrolidin-2-yl)-1H-pyrrolo[3,2-c]pyridine-1-carboxylate (Intermediate 492, 0.47 g, 90%) as a yellow solid. ¹H NMR (400 MHz, DMSO-d₆) δ ppm −0.03 (s, 9H), 0.82-0.86 (m, 2H), 1.60-1.65 (m, 2H), 1.69 (s, 9H), 1.72-1.81 (m, 4H), 2.01-2.09 (m, 1H), 2.29-2.32 (m, 2H), 2.39 (m, 3H), 2.55-2.60 (m, 2H), 2.64-2.75 (m, 4H), 3.00-3.16 (m, 3H), 3.48-3.56 (m, 2H), 3.88-3.94 (m, 1H), 4.25-4.30 (m, 1H), 4.42-4.46 (m, 1H), 5.02-5.08 (m, 2H), 5.21-5.27 (m, 1H), 6.74 (s, 1H), 7.19 (t, J=7.60, 2H), 7.56-7.62 (m, 1H), 7.65-7.69 (m, 1H), 7.73 (d, J=6.0 Hz, 1H), 8.55 (s, 1H), 8.91 (s, 1H), 10.48 (br s, 1H). Mass spec m/z 909.4 [M+H]⁺.

### Step 3

### Example 114: 4-(6-(2-(2,6-dioxopiperidin-3-yl)-6-fluoro-1-oxoisoindolin-5-yl)hex-5-yn-1-yl)-2-fluoro-N-(2-((R)-1-methylpyrrolidin-2-yl)-1H-pyrrolo[3,2-c]pyridin-6-yl)benzamide

To a solution of tert-butyl 6-(4-(6-(2-(2,6-dioxo-1-((2-(trimethylsilyl)ethoxy)methyl)piperidin-3-yl)-6-fluoro-1-oxoisoindolin-5-yl)hex-5-yn-1-yl)-2-fluorobenzamido)-2-((R)-1-methylpyrrolidin-2-yl)-1H-pyrrolo[3,2-c]pyridine-1-carboxylate (Intermediate 492, 0.47 g, 0.51 mmol) in acetonitrile (10 mL) was added methanesulfonic acid (0.34 mL, 5.17 mmol) and the reaction mixture was heated at 50° C. for 2 h. After cooling to room temperature, N,N′-dimethylethylenediamine (0.31 mL, 2.58 mmol) and triethylamine (1.45 mL, 10.34 mmol) were added and the reaction stirred at room temperature for 3 h. The reaction mixture was concentrated in vacuo and water (50 mL) added. The resultant precipitate was collected by filtration and dried in vacuo. The crude material was purified by preparative HPLC (Method A) to afford 4-(6-(2-(2,6-dioxopiperidin-3-yl)-6-fluoro-1-oxoisoindolin-5-yl)hex-5-yn-1-yl)-2-fluoro-N-(2-((R)-1-methylpyrrolidin-2-yl)-1H-pyrrolo[3,2-c]pyridin-6-yl)benzamide (Example 114, 0.05 g, 15%) as an off-white solid. ¹H NMR (400 MHz, DMSO-d₆) δ ppm 1.59-1.67 (m, 2H), 1.75-1.91 (m, 5H), 1.95-2.05 (m, 1H), 2.08-2.14 (m, 1H), 2.16 (s, 3H), 2.24-2.28 (m, 1H), 2.32-2.38 (m, 1H), 2.45-2.48 (m, 1H), 2.52-2.58 (m, 1H), 2.57-2.62 (m, 2H), 2.73 (t, J=7.60 Hz, 2H), 2.83-2.95 (m, 1H), 3.11-3.16 (m, 1H), 4.24-4.34 (m, 1H), 4.38-4.46 (m, 1H), 5.08-5.13 (m, 1H), 6.39 (s, 1H), 7.18 (t, J=7.60 Hz, 2H), 7.56 (d, J=7.60 Hz, 1H), 7.66-7.73 (m, 2H), 8.19 (s, 1H), 8.46 (s, 1H), 10.20 (d, J=4.40 Hz, 1H), 10.99 (br s, 1H), 11.37 (br s, 1H). Mass spec m/z 679.0 [M+H]⁺.

### Example 115 was Prepared Following Scheme 114

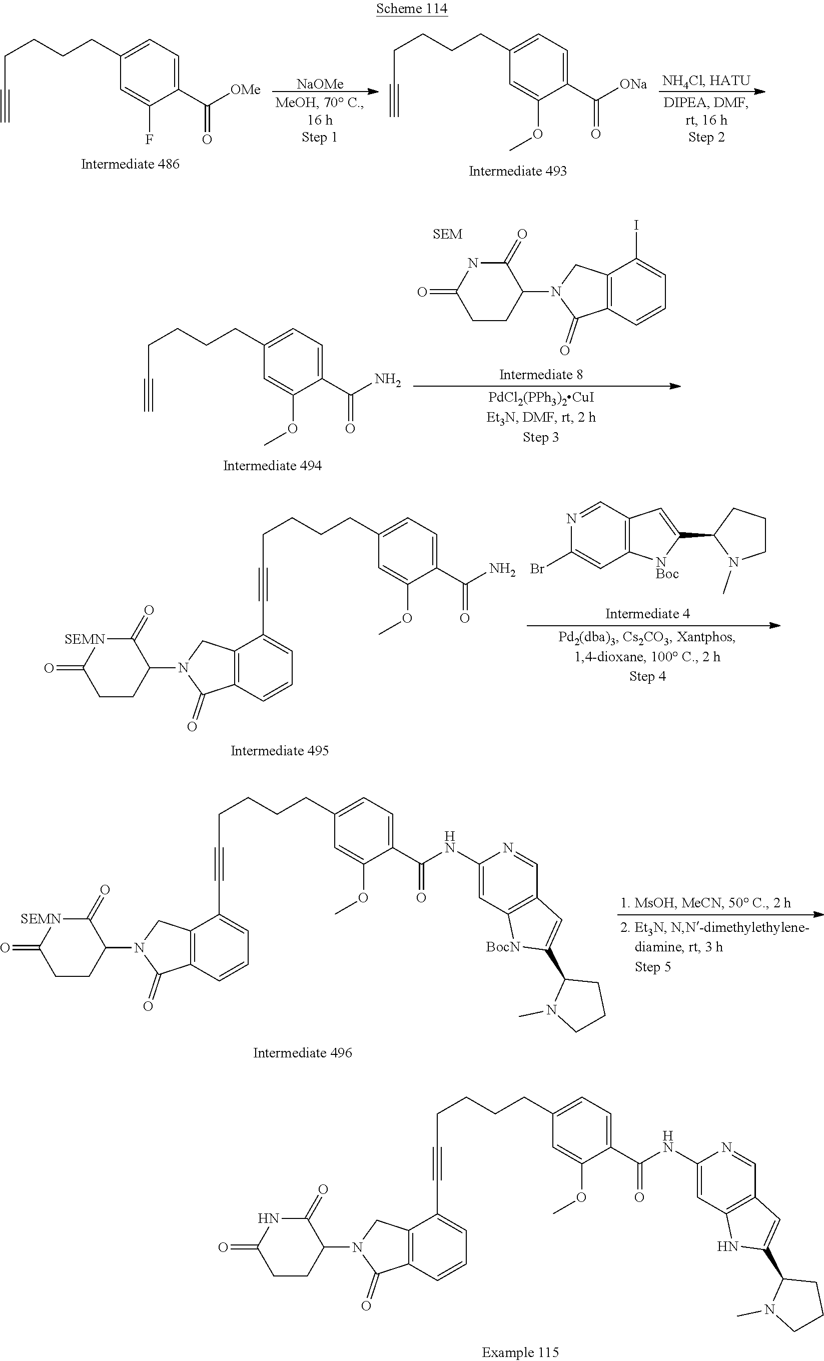

### Step 1

### Intermediate 493: Sodium salt of 4-(hex-5-yn-1-yl)-2-methoxybenzoic acid

To a solution of methyl 2-fluoro-4-(hex-5-yn-1-yl)benzoate (Intermediate 486, 2.0 g, 8.53 mmol) in methanol (20 mL) was added sodium methoxide (1.42 g, 25.61 mmol) and the reaction mixture was heated at 70° C. for 16 h. The reaction mixture was then concentrated in vacuo to afford the sodium salt of 4-(hex-5-yn-1-yl)-2-methoxybenzoic acid (Intermediate 493, 1.8 g, 81%) as a white solid, which was used for the next step without further purification. ¹H NMR (400 MHz, DMSO-d₆) δ ppm 1.42-1.49 (m, 2H), 1.64-1.69 (m, 2H), 2.16-2.21 (m, 2H), 2.65 (t, J=7.64 Hz, 2H), 2.77 (t, J=2.63 Hz, 1H), 3.37 (s, 3H), 7.12-7.19 (m, 2H), 7.76-7.81 (m, 1H), 13.08 (br s, 1H). Mass spec m/z 233.0 [M+H]⁺.

### Step 2

### Intermediate 494: 4-(hex-5-yn-1-yl)-2-methoxybenzamide

To a cooled (0° C.) solution of the sodium salt of 4-(hex-5-yn-1-yl)-2-methoxybenzoic acid (Intermediate 493, 1.6 g, 6.9 mmol) in dimethylformamide (20 mL) was added HATU (4.1 g, 10.0 mmol) and N,N-diisopropylethylamine (4.9 mL, 28.0 mmol). Ammonium chloride (1.5 g, 28.0 mmol) was added and the reaction stirred at room temperature for 16 h. The reaction mixture was poured in ice cold water (150 mL), the resultant precipitate was collected by filtration and dried in vacuo to afford 4-(hex-5-yn-1-yl)-2-methoxybenzamide (Intermediate 494, 1.4 g, 62%) as a white solid, which was used for the next step without further purification. ¹H NMR (400 MHz, DMSO-d₆) δ ppm 1.41-1.48 (m, 2H), 1.62-1.67 (m, 2H), 2.16-2.19 (m, 2H), 2.63 (t, J=7.46 Hz, 2H), 2.75 (s, 1H), 3.83 (s, 3H), 7.07-7.12 (m, 2H), 7.55-7.61 (m, 3H). Mass spec m/z 232.0 [M+H]⁺.

### Step 3

### Intermediate 495: 4-(6-(2-(2,6-dioxo-1-((2-(trimethylsilyl)ethoxy)methyl)piperidin-3-yl)-1-oxoisoindolin-4-yl)hex-5-yn-1-yl)-2-methoxybenzamide

To a solution of 4-(hex-5-yn-1-yl)-2-methoxybenzamide (Intermediate 494, 0.5 g, 2.16 mmol) in dimethylformamide (10 mL) was added 3-(4-iodo-1-oxoisoindolin-2-yl)-1-((2-(trimethylsilyl)ethoxy)methyl)piperidine-2,6-dione (Intermediate 8, 1.08 g, 2.16 mmol) followed by triethylamine (10.9 mL, 77.82 mmol). The reaction mixture was purged with argon for 15 min then copper (I) iodide (0.04 g, 0.21 mmol) and PdCl₂(PPh₃)₂ (0.15 g, 0.21 mmol) were added. The reaction mixture was further purged with argon for 10 min and stirred at room temperature for 2 h. After completion, the reaction mixture was diluted with water (300 mL) and extracted with ethyl acetate (3×200 mL). The combined organic layers were dried over anhydrous Na₂SO₄ and concentrated in vacuo. The crude material was purified by combi-flash chromatography, by eluting with 28% ethyl acetate in heptane, to afford 4-(6-(2-(2,6-dioxo-1-((2-(trimethylsilyl)ethoxy)methyl)piperidin-3-yl)-1-oxoisoindolin-4-yl)hex-5-yn-1-yl)-2-methoxybenzamide (Intermediate 495, 0.9 g, 69%) as a yellow solid. ¹H NMR (400 MHz, DMSO-d₆) δ ppm −0.04 (s, 9H), 0.82-0.85 (m, 2H), 1.56-1.61 (m, 2H), 1.72-1.76 (m, 2H), 2.01-2.11 (m, 1H), 2.29-2.43 (m, 2H), 2.51-2.56 (m, 2H), 2.67-2.90 (m, 3H), 3.04-3.10 (m, 1H), 3.32 (s, 3H), 3.49-3.54 (m, 2H), 4.23-4.27 (m, 1H), 4.43-4.47 (m, 1H), 5.01-5.08 (m, 1H), 5.23-5.30 (m, 1H), 7.11 (d, J=7.20 Hz, 2H), 7.15 (s, 1H), 7.50-7.57 (m, 2H), 7.59-7.62 (m, 1H), 7.71 (br s, 1H), 7.73 (br s, 1H).

### Step 4

### Intermediate 496: tert-butyl 6-(4-(6-(2-(2,6-dioxo-1-((2-(trimethylsilyl)ethoxy)methyl)piperidin-3-yl)-1-oxoisoindolin-4-yl)hex-5-yn-1-yl)-2-methoxybenzamido)-2-((R)-1-methylpyrrolidin-2-yl)-1H-pyrrolo[3,2-c]pyridine-1-carboxylate

To a solution of 4-(6-(2-(2,6-dioxo-1-((2-(trimethylsilyl)ethoxy)methyl)piperidin-3-yl)-1-oxoisoindolin-4-yl)hex-5-yn-1-yl)-2-methoxybenzamide (Intermediate 495, 0.56 g, 0.92 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 0.42 g, 1.11 mmol) followed by cesium carbonate (0.90 g, 2.78 mmol). The reaction mixture was purged with argon for 15 min followed by the addition of Pd₂(dba)₃ (0.13 g, 0.13 mmol) and Xantphos (0.16 g, 0.27 mmol). The reaction mixture was further purged with argon for 10 min and then heated at 100° C. for 2 h. After completion, the reaction mixture was filleted through a celite bed, the filter pad was washed with ethyl acetate (500 mL) and the filtrate concentrated in vacuo. The crude material was purified by combi-flash chromatography, by eluting with 90% ethyl acetate in heptane, to afford tert-butyl 6-(4-(6-(2-(2,6-dioxo-1-((2-(trimethylsilyl)ethoxy)methyl)piperidin-3-yl)-1-oxoisoindolin-4-yl)hex-5-yn-1-yl)-2-methoxybenzamido)-2-((R)-1-methylpyrrolidin-2-yl)-1H-pyrrolo[3,2-c]pyridine-1-carboxylate (Intermediate 496, 0.44 g, 52%) as a yellow solid. ¹H NMR (400 MHz, DMSO-d₆) δ ppm −0.05 (s, 9H), 0.76-0.86 (m, 2H), 1.56-1.65 (m, 2H), 1.70 (s, 9H), 1.73-1.83 (m, 3H), 2.01-2.10 (m, 3H), 2.35 (s, 3H), 2.37-2.42 (m, 2H), 2.52-2.57 (m, 2H), 2.67-2.74 (m, 3H), 3.02-3.12 (m, 2H), 3.47-3.52 (m, 2H), 3.89-3.94 (m, 2H), 3.99 (s, 3H), 4.23-4.27 (m, 1H), 4.43-4.47 (m, 1H), 4.99-5.06 (m, 2H), 5.23-5.27 (m, 1H), 6.74 (s, 1H), 7.00 (d, J=7.88 Hz, 1H), 7.12 (s, 1H), 7.20-7.41 (m, 3H), 7.53 (t, J=7.60 Hz, 1H), 7.65 (d, J=7.88 Hz, 1H), 7.72 (d, J=7.60 Hz, 1H), 10.45 (br s, 1H). Mass spec m/z 903.6 [M+H]⁺.

### Step 5

### Example 115: 4-(6-(2-(2,6-dioxopiperidin-3-yl)-1-oxoisoindolin-4-yl)hex-5-yn-1-yl)-2-methoxy-N-(2-((R)-1-methylpyrrolidin-2-yl)-1H-pyrrolo[3,2-c]pyridin-6-yl)benzamide

To a solution of tert-butyl 6-(4-(6-(2-(2,6-dioxo-1-((2-(trimethylsilyl) ethoxy)methyl)piperidin-3-yl)-1-oxoisoindolin-4-yl)hex-5-yn-1-yl)-2-methoxybenzamido)-2-((R)-1-methylpyrrolidin-2-yl)-1H-pyrrolo[3,2-c]pyridine-1-carboxylate (Intermediate 496, 0.41 g, 0.45 mmol) in acetonitrile (10 mL) was added methanesulfonic acid (0.30 mL, 4.53 mmol) and the reaction was heated at 50° C. for 2 h. After cooling to room temperature, N,N′-dimethylethylenediamine (0.27 mL, 2.27 mmol) followed by triethylamine (1.27 mL, 9.07 mmol) were added and the reaction mixture was stirred at room temperature for 3 h. The reaction mixture was quenched with water (50 mL), the resultant precipitate was collected by filtration and dried in vacuo. The crude material was purified by preparative HPLC (Method A) to afford 4-(6-(2-(2,6-dioxopiperidin-3-yl)-1-oxoisoindolin-4-yl)hex-5-yn-1-yl)-2-methoxy-N-(2-((R)-1-methylpyrrolidin-2-yl)-1H-pyrrolo[3,2-c]pyridin-6-yl)benzamide (Example 115, 0.04 g, 15%) as an off-white solid. ¹H NMR (400 MHz, DMSO-d₆) δ ppm 1.59-1.66 (m, 2H), 1.78-1.93 (m, 5H), 1.97-2.05 (m, 1H), 2.10-2.15 (m, 1H), 2.15 (s, 3H), 2.23-2.29 (m, 1H), 2.44-2.48 (m, 1H), 2.54-2.59 (m, 3H), 2.73 (t, J=7.58 Hz, 2H), 2.86-2.97 (m, 1H), 3.11-3.18 (m, 1H), 3.28-3.32 (m, 1H), 4.02 (s, 3H), 4.27-4.35 (m, 1H), 4.41-4.48 (m, 1H), 5.10-5.11 (m, 1H), 6.38 (s, 1H), 7.01 (d, J=8.07 Hz, 1H), 7.13 (s, 1H), 7.50-7.56 (m, 1H), 7.65 (d, J=6.97 Hz, 1H), 7.71 (d, J=7.46 Hz, 1H), 7.97 (d, J=7.95 Hz, 1H), 8.27 (s, 1H), 8.45 (s, 1H), 10.31 (br s, 1H), 11.02 (br s, 1H), 11.39 (br s, 1H). Mass spec m/z 673.0 [M+H]⁺.

### Example 116 was Synthesised Following Scheme 115

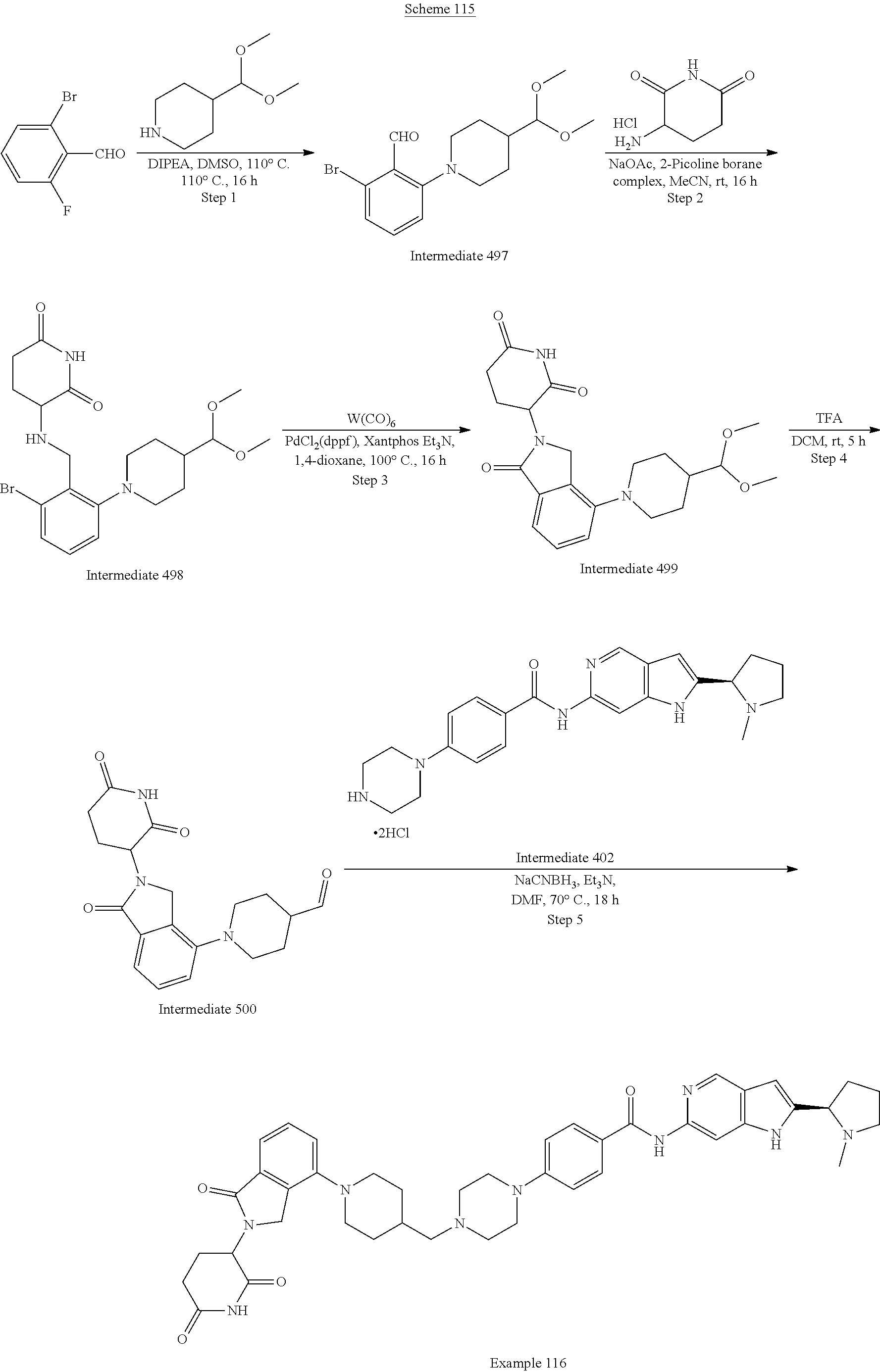

### Step 1

### Intermediate 497: 2-bromo-6-(4-(dimethoxymethyl)piperidin-1-yl)benzaldehyde

To a solution of 2-bromo-6-fluorobenzaldehyde (CAS No. 360575-28-6, 5.0 g, 24.62 mmol) in dimethyl sulfoxide (50 mL) was added 4-(dimethoxymethyl)piperidine (CAS No. 188646-83-5, 3.92 g, 24.62 mmol) and N,N-diisopropylethylamine (12.9 mL, 73.88 mmol). The reaction mixture was heated at 110° C. for 16 h. After completion, reaction mixture was diluted with water (200 mL) and extracted with ethyl acetate (2×200 mL). The combined organic layers were dried over anhydrous Na₂SO₄, filtered, and concentrated in vacuo to afford 2-bromo-6-(4-(dimethoxymethyl)piperidin-1-yl)benzaldehyde (Intermediate 497, 4.0 g, 38%) as a yellow solid, which was used for the next step without further purification. ¹H NMR (400 MHz, DMSO-d₆) δ ppm 1.40-1.43 (m, 2H), 1.69-1.72 (m, 2H), 2.78-2.84 (m, 1H), 3.03-3.24 (m, 2H), 3.15-3.18 (m, 2H), 3.27 (s, 6H), 4.10-4.14 (m, 1H), 7.21 (d, J=8.19 Hz, 1H), 7.31 (d, J=7.70 Hz, 1H), 7.41 (t, J=8.01 Hz, 1H), 10.01 (br s, 1H). Mass spec m/z 344.0 [M+H+2]⁺.

### Step 2

### Intermediate 498: 3-((2-bromo-6-(4-(dimethoxymethyl)piperidin-1-yl)benzyl)amino)piperidine-2,6-dione

To a solution of 2-bromo-6-(4-(dimethoxymethyl)piperidin-1-yl)benzaldehyde (Intermediate 497, 2.0 g, 5.8 mmol) in acetonitrile (20 mL) was added 3-aminopiperidine-2,6-dione hydrochloride (CAS No. 24666-56-6, 0.96 g, 5.8 mmol) and sodium acetate (0.73 g, 8.8 mmol), followed by 2-picoline borane complex (1.3 g, 12.0 mmol). The reaction mixture was stirred at room temperature for 16 h. After completion, reaction mixture was filtered through a celite bed and the filter pad washed with ethyl acetate (50 mL). The filtrate was concentrated in vacuo and crude material was purified by combi-flash chromatography, by eluting with 5% MeOH in DCM, to afford 3-((2-bromo-6-(4-(dimethoxymethyl)piperidin-1-yl)benzyl)amino)piperidine-2,6-dione (Intermediate 498, 1.7 g, 65%) as a yellow solid. ¹H NMR (400 MHz, DMSO-d₆) δ ppm 1.35-1.44 (m, 2H), 1.67-1.76 (m, 4H), 2.27-2.30 (m, 1H), 2.41-2.48 (m, 1H), 2.52-2.60 (m, 2H), 2.69-2.75 (m, 1H), 3.06-3.10 (m, 1H), 3.20-3.27 (m, 2H), 3.28 (s, 6H), 3.85-3.95 (m, 2H), 4.10-4.15 (m, 1H), 7.12-7.20 (m, 2H), 7.33 (d, J=7.43 Hz, 1H), 10.78 (br s, 1H). Mass spec m/z 454.0 [M+H]⁺.

### Step 3

### Intermediate 499: 3-(4-(4-(dimethoxymethyl)piperidin-1-yl)-1-oxoisoindolin-2-yl)piperidine-2,6-dione

To a solution of 3-((2-bromo-6-(4-(dimethoxymethyl)piperidin-1-yl)benzyl)amino)piperidine-2,6-dione (Intermediate 498, 1.0 g, 2.21 mmol) in 1,4-dioxane (10 mL) was added triethylamine (0.92 mL, 6.60 mmol). The reaction mixture was purged with argon for 15 min then Xantphos (0.26 g, 0.44 mmol) followed by PdCl₂(dppf) (0.33 g, 0.44 mmol) and tungsten hexacarbonyl (0.39 g, 1.10 mmol) were added. The reaction mixture was further purged with argon for 10 min and heated at 100° C. for 16 h. After completion, the reaction mixture was concentrated in vacuo. The crude material was purified by combi-flash chromatography, by eluting with 5% MeOH in DCM, to afford 3-(4-(4-(dimethoxymethyl)piperidin-1-yl)-1-oxoisoindolin-2-yl)piperidine-2,6-dione (Intermediate 499, 0.8 g, 90%) as a brown solid. ¹H NMR (400 MHz, DMSO-d₆) δ ppm 1.32-1.40 (m, 2H), 1.71-1.75 (m, 3H), 1.97-2.00 (m, 1H), 2.52-2.63 (m, 2H), 2.67-2.73 (m, 2H), 2.87-2.96 (m, 1H), 3.28 (s, 6H), 3.38-3.41 (m, 2H), 4.09-4.11 (m, 1H), 4.22-4.29 (m, 1H), 4.38-4.44 (m, 1H), 5.08-5.13 (m, 1H), 7.15 (d, J=7.88 Hz, 1H), 7.29 (d, J=7.46 Hz, 1H), 7.40-7.44 (m, 1H), 10.98 (s, 1H). Mass spec m/z 402.1 [M+H]⁺.

### Step 4

### Intermediate 500: 1-(2-(2,6-dioxopiperidin-3-yl)-1-oxoisoindolin-4-yl)piperidine-4-carbaldehyde

To a solution of 3-(4-(4-(dimethoxymethyl)piperidin-1-yl)-1-oxoisoindolin-2-yl)piperidine-2,6-dione (Intermediate 499, 1.5 g, 3.7 mmol) in dichloromethane (20 mL) was added trifluoroacetic acid (18 mL) at room temperature. The reaction mixture was stirred at room temperature for 5 h. After completion, the reaction mixture was concentrated in vacuo to afford 1-(2-(2,6-dioxopiperidin-3-yl)-1-oxoisoindolin-4-yl)piperidine-4-carbaldehyde (Intermediate 500, 1.0 g, 75%) as a yellow solid, which was used for the next step without further purification. ¹H NMR (400 MHz, DMSO-d₆) δ ppm 1.64-1.70 (m, 2H), 1.96-2.05 (m, 3H), 2.40-2.45 (m, 2H), 2.58-2.61 (m, 1H), 2.81-2.95 (m, 3H), 3.30-3.37 (m, 2H), 4.28-4.23 (m, 1H), 4.42-4.47 (m, 1H), 5.09-5.14 (m, 1H), 7.19 (d, J=7.83 Hz, 1H), 7.33 (d, J=7.43 Hz, 2H), 9.67 (s, 1H), 10.98 (br s, 1H). Mass spec m/z 356.1 [M+H]⁺.

### Step 5

### Example 116: 4-(4-((1-(2-(2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)benzamide

To a solution of 1-(2-(2,6-dioxopiperidin-3-yl)-1-oxoisoindolin-4-yl)piperidine-4-carbaldehyde (Intermediate 500, 0.5 g, 1.27 mmol) in dimethylformamide (4 mL) were added triethylamine (0.52 mL, 3.82 mmol) followed by (R)—N-(2-(1-methylpyrrolidin-2-yl)-1H-pyrrolo[3,2-c]pyridin-6-yl)-4-(piperazin-1-yl)benzamide dihydrochloride (Intermediate 402, 0.51 g, 1.27 mmol) and the reaction mixture was heated at 70° C. for 2 h. Sodium cyanoborohydride (0.16 g, 2.55 mmol) was then added and the reaction mixture was heated at 70° C. for 16 h. After completion, the reaction mixture was diluted with ice cold water (30 mL) and stirred for 10 min.

The resultant precipitate was collected by filtration and dried in vacuo. The crude material was purified by preparative HPLC (Method A) to afford 4-(4-((1-(2-(2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)benzamide (Example 116, 0.08 g, 8%) as an off-white solid. ¹H NMR (400 MHz, DMSO-d₆) δ ppm 1.25-1.34 (m, 2H), 1.74-2.00 (m, 7H), 2.07-2.10 (m, 1H), 2.11-2.15 (m, 1H), 2.16 (s, 3H), 2.21-2.30 (m, 3H), 2.55-2.64 (m, 2H), 2.70-2.81 (m, 3H), 2.84-2.99 (m, 2H), 3.06-3.10 (m, 1H), 3.11-3.17 (m, 1H), 3.25-3.31 (m, 5H), 3.34-3.43 (m, 2H), 4.27-4.31 (m, 1H), 4.41-4.45 (m, 1H), 5.09-5.13 (m, 1H), 6.37 (s, 1H), 6.98 (d, J=9.13 Hz, 2H), 7.16 (d, J=7.75 Hz, 1H), 7.28-7.34 (m, 1H), 7.43 (t, J=7.17 Hz, 1H), 7.96 (d, J=9.01 Hz, 2H), 8.17 (s, 1H), 8.47 (s, 1H), 10.09 (br s, 1H), 10.98 (br s, 1H), 11.32 (br s, 1H). Mass spec m/z 744.0 [M+H]⁺.

### Examples 117 and 118 were prepared according to Scheme 116

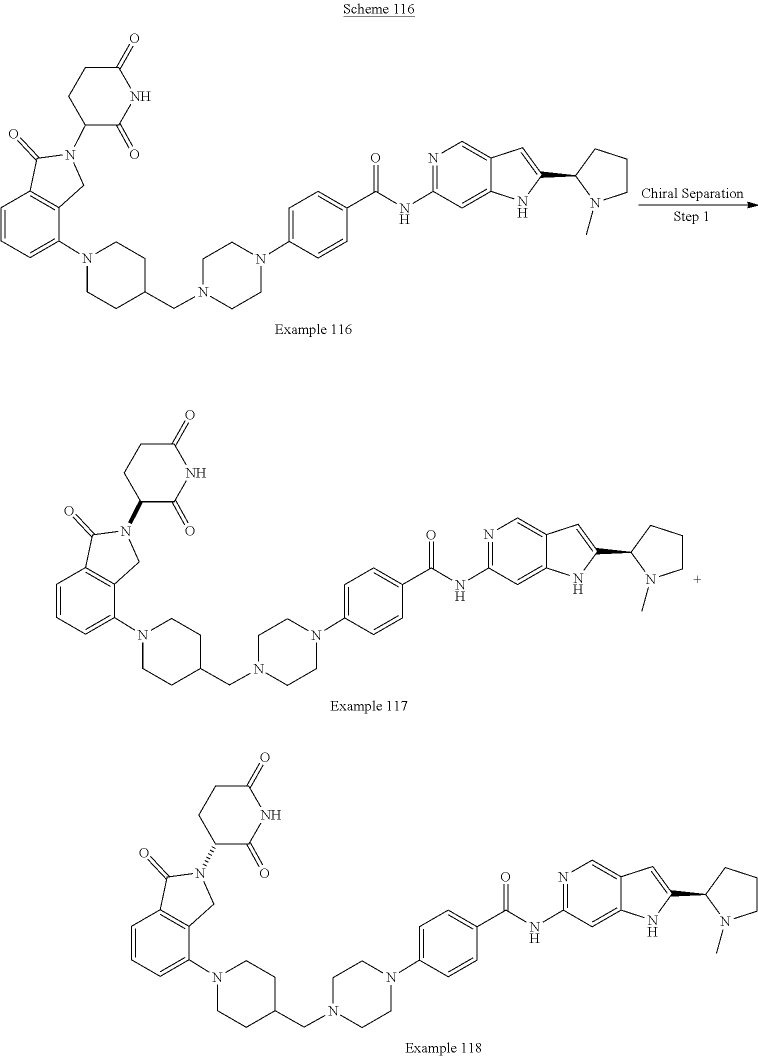 [SMILES: CN1CCC[C@@H]1c1cc2cnc(NC(=O)c3ccc(N4CCN(CC5CCN(c6cccc7c6CN(C6CCC(=O)NC6=O)C7=O)CC5)CC4)cc3)cc2[nH]1 ; CN1CCC[C@@H]1c1cc2cnc(NC(=O)c3ccc(N4CCN(CC5CCN(c6cccc7c6CN([C@H]6CCC(=O)NC6=O)C7=O)CC5)CC4)cc3)cc2[nH]1 ; CN1CCC[C@@H]1c1cc2cnc(NC(=O)c3ccc(N4CCN(CC5CCN(c6cccc7c6CN([C@@H]6CCC(=O)NC6=O)C7=O)CC5)CC4)cc3)cc2[nH]1]

### Step 1

### Example 117: 4-(4-((1-(2-((rel-S)-2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)benzamide and Example 118: 4-(4-((1-(2-((rel-R)-2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)benzamide

4-(4-((1-(2-(2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)benzamide (Example 116, 0.05 g, 0.06 mmol) was separated by chiral preparative HPLC (Column: IC (20×250*mm), 5 m; Mobile Phase A: DCM, Mobile Phase B: DCM:iPrOH (1:1); Flow rate: 18 ml/min; isocratic 50% B) to afford 4-(4-((1-(2-((rel-S)-2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)benzamide (Example 117, 1st eluting peak, 0.006 g, 16%) as an off-white solid and 4-(4-((1-(2-((rel-R)-2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)benzamide (Example 118, 2nd eluting peak, 0.009 g, 24%) as an off-white solid.

### Example 117: 4-(4-((1-(2-((rel-S)-2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)benzamide

1^{st} eluting peak from chiral preparative HPLC. Retention time: 7.21 min.

¹H NMR (400 MHz, DMSO-d₆) δ ppm 1.23-1.33 (m, 3H), 1.69-1.79 (m, 2H), 1.83-1.89 (m, 3H), 1.97-2.00 (m, 2H), 2.12-2.21 (m, 4H), 2.22-2.29 (m, 3H), 2.54-2.55 (m, 2H), 2.56-2.60 (m, 2H), 2.70-2.81 (m, 3H), 2.86-2.96 (m, 2H), 3.11-3.19 (m, 1H), 3.27-3.30 (m, 4H), 3.38-3.45 (m, 2H), 4.27-4.33 (m, 1H), 4.41-4.47 (m, 1H), 5.09-5.13 (m, 1H), 6.37 (s, 1H), 6.98 (d, J=8.93 Hz, 2H), 7.16 (d, J=7.82 Hz, 1H), 7.27 (d, J=7.34 Hz, 1H), 7.41-7.46 (m, 1H), 7.96 (d, J=8.68 Hz, 2H), 8.17 (s, 1H), 8.47 (s, 1H), 10.07 (br s, 1H), 10.99 (br s, 1H), 11.36 (br s, 1H). Mass spec m/z 744.5 [M+H]⁺.

### Example 118: 4-(4-((1-(2-((rel-R)-2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)benzamide

2nd eluting peak from chiral preparative HPLC. Retention time: 8.99 min.

¹H NMR (400 MHz, DMSO-d₆) δ ppm 1.23-1.33 (m, 3H), 1.69-1.79 (m, 2H), 1.83-1.89 (m, 3H), 1.97-2.03 (m, 2H), 2.12-2.21 (m, 4H), 2.22-2.29 (m, 3H), 2.54-2.55 (m, 2H), 2.56-2.60 (m, 2H), 2.70-2.81 (m, 3H), 2.86-2.96 (m, 2H), 3.11-3.19 (m, 1H), 3.27-3.30 (m, 4H), 3.38-3.45 (m, 2H), 4.26-4.33 (m, 1H), 4.41-4.47 (m, 1H), 5.09-5.14 (m, 1H), 6.38 (s, 1H), 6.99 (d, J=8.93 Hz, 2H), 7.17 (d, J=7.82 Hz, 1H), 7.30 (d, J=7.34 Hz, 1H), 7.40-7.46 (m, 1H), 7.96 (d, J=8.68 Hz, 2H), 8.17 (s, 1H), 8.48 (s, 1H), 10.11 (br s, 1H), 10.98 (br s, 1H), 11.33 (br s, 1H). Mass spec m/z 744.2 [M+H]⁺.

### Example 119 was Synthesised Following Scheme 117

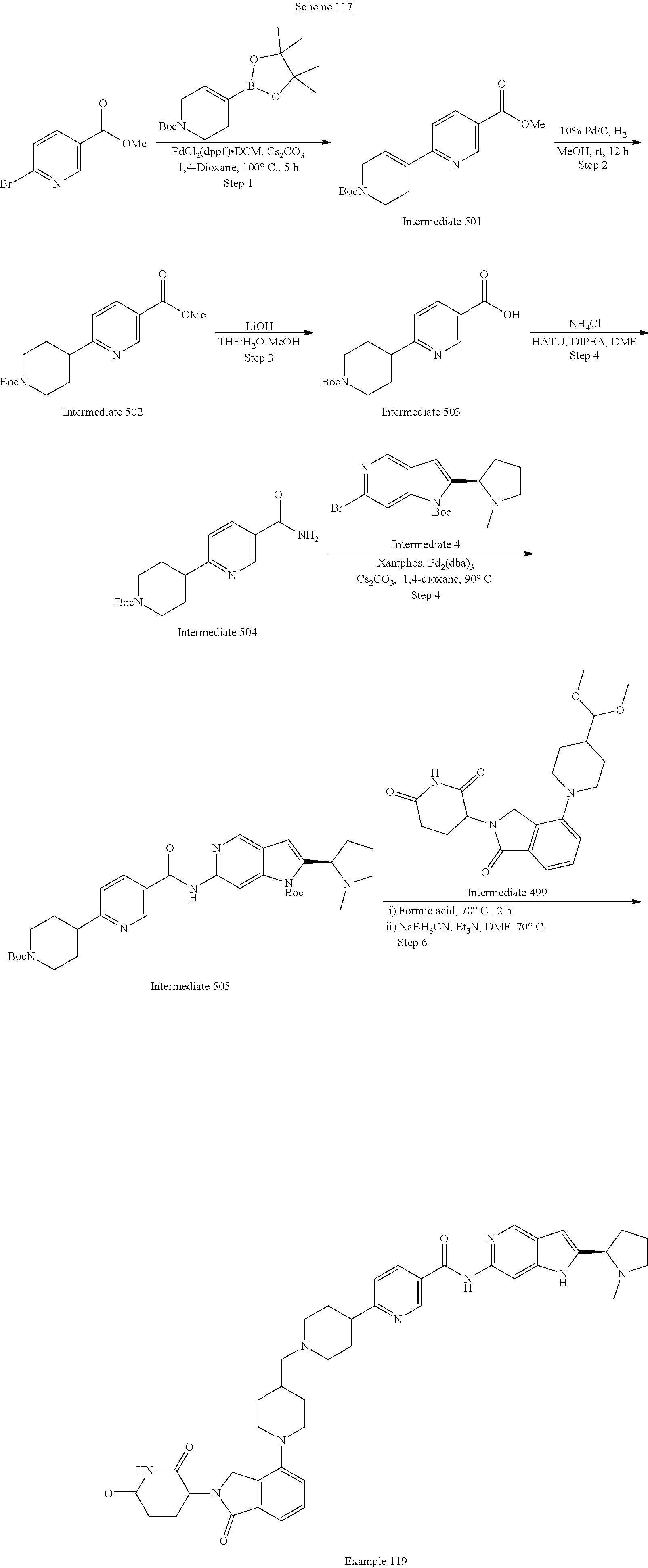

### Step 1

### Intermediate 501: 1′-(tert-butyl) 5-methyl 3′,6′-dihydro-[2,4′-bipyridine]-1′,5(2′H)-dicarboxylate

To a solution of methyl 6-bromonicotinate (CAS No. 26218-78-0, 4.3 g, 19.9 mmol) in 1,4-dioxane (15 mL) was added tert-butyl 4-(4,4,5,5-tetramethyl-1,3,2-dioxaborolan-2-yl)-3,6-dihydropyridine-1(2H)-carboxylate (CAS No. 286961-14-6, 9.23 g, 29.9 mmol) and cesium carbonate (7.68 g, 39.8 mmol). The reaction mixture was purged with argon gas for 15 min, then Pd(dppf)Cl₂·DCM (1.68 g, 1.99 mmol) was added. The resulting reaction was purged with argon for another 10 min and stirred for 100° C. for 5 h. The reaction mixture was diluted with water (200 mL) and extracted with ethyl acetate (2×200 mL). The combined organic layers were dried over anhydrous Na₂SO₄ and concentrated in vacuo. The crude material was purified by combi-flash chromatography, by eluting with 20% ethyl acetate in heptane, to afford 1′-(tert-butyl) 5-methyl 3′,6′-dihydro-[2,4′-bipyridine]-1′,5(2′H)-dicarboxylate (Intermediate 501, 3.8 g, 60%) as white solid. ¹H NMR (400 MHz, DMSO-d₆) δ ppm 1.43 (s, 9H), 2.57-2.61 (m, 2H), 3.52-3.56 (m, 2H), 3.87 (s, 3H), 4.06-4.10 (m, 2H), 6.89 (s, 1H), 7.70 (d, J=8.4 Hz, 1H), 8.24 (d, J=8.4 Hz, 1H), 9.03 (s, 1H). Mass spec m/z 319.3 [M+H]⁺.

### Step 2

### Intermediate 502: methyl 6-(1-(tert-butoxycarbonyl)piperidin-4-yl)nicotinate

To a solution of 1′-(tert-butyl) 5-methyl 3′,6′-dihydro-[2,4′-bipyridine]-1′,5(2′H)-dicarboxylate (Intermediate 501, 7.7 g, 30 mmol) in methanol (80 mL), was added 10% Pd/C (0.525 g) and the reaction mixture was stirred at room temperature for 16 h under 30 psi H₂ atmosphere. The reaction mixture was filtered through a celite bed, the filter pad was washed with ethyl acetate (100 mL) and the filtrate concentrated in vacuo to afford methyl 6-(1-(tert-butoxycarbonyl)piperidin-4-yl)nicotinate (Intermediate 502, 7.0 g, 89%) as a dark liquid which was directly used in next step without further purification. ¹H NMR (400 MHz, DMSO-d₆) δ ppm 1.38 (s, 9H), 1.45-1.60 (m, 2H), 1.76-1.81 (m, 2H), 2.80-2.97 (m, 3H), 3.84 (s, 3H), 4.01-4.05 (m, 2H), 7.43 (d, J=8.00 Hz, 1H), 8.16-8.19 (m, 1H), 8.98 (s, 1H). Mass spec m/z 321.0 [M+H]⁺.

### Step 3

### Intermediate 503: 6-(1-(tert-butoxycarbonyl)piperidin-4-yl)nicotinic acid

To a solution of methyl 6-(1-(tert-butoxycarbonyl)piperidin-4-yl)nicotinate (Intermediate 502, 4.0 g, 12.48 mmol) in tetrahydrofuran (15 mL), methanol (15 mL) and water (15 mL) was added lithium hydroxide (1.22 g, 49.9 mmol) and the reaction mixture was stirred at room temperature for 16 h. The reaction mixture was acidified to pH~4 by the addition of aqueous citric acid solution. The resultant precipitate was collected by filtration and dried in vacuo to afford 6-(1-(tert-butoxycarbonyl)piperidin-4-yl)nicotinic acid (Intermediate 503, 5.0 g) as off-white solid, which was used for next step without further purification. ¹H NMR (400 MHz, DMSO-d₆) δ ppm 1.41 (s, 9H), 1.57-1.63 (s, 2H), 1.81-1.85 (m, 2H), 2.80-2.98 (m, 3H), 4.01-4.08 (m, 2H), 7.43 (d, J=8.0 Hz, 1H), 8.17 (d, J=8.0 Hz, 1H), 8.99 (s, 1H), 13.13 (bs, 1H).

### Step 4

### Intermediate 504: tert-butyl 4-(5-carbamoylpyridin-2-yl)piperidine-1-carboxylate

To a solution of 6-(1-(tert-butoxycarbonyl)piperidin-4-yl)nicotinic acid (Intermediate 503, 5.0 g, 16.3 mmol) in dimethylformamide (30 mL) were added N,N-diisopropylethylamine (8.53 mL, 49.0 mmol) and HATU (12.39 g, 32.6 mmol). The reaction mixture was stirred at room temperature for 15 min, then ammonium chloride (4.36 g, 81.6 mmol) was added, and the reaction mixture was stirred at room temperature for 16 h. The reaction mixture was then quenched with ice cold water (200 mL), the resultant precipitate was collected by filtration and dried in vacuo to afford tert-butyl 4-(5-carbamoylpyridin-2-yl)piperidine-1-carboxylate (Intermediate 504, 3.0 g, 51%) as a white solid. Mass spec m/z 304.0 [M−H]⁻.

### Step 5

### Intermediate 505: tert-butyl (R)-6-(6-(1-(tert-butoxycarbonyl)piperidin-4-yl)nicotinamido)-2-(1-methylpyrrolidin-2-yl)-1H-pyrrolo[3,2-c]pyridine-1-carboxylate

To a solution of tert-butyl 4-(5-carbamoylpyridin-2-yl)piperidine-1-carboxylate (Intermediate 504, 0.60 g, 1.66 mmol) in 1,4-dioxane (30 mL) was added tert-butyl (R)-6-bromo-2-(1-methylpyrrolidin-2-yl)-1H-pyrrolo[3,2-c]pyridine-1-carboxylate (Intermediate 4, 0.69 g, 1.82 mmol) and cesium carbonate (1.62 g, 4.97 mmol). The reaction mixture was purged with argon for 15 min, then Xanthphos (0.29 g, 0.50 mmol) and Pd₂(dba)₃ (0.23 g, 0.25 mmol) were added. The reaction was purged with argon for another 10 min and heated at 100° C. for 2 h. The reaction mixture was filtered through a celite bed, the filter pad was washed with ethyl acetate (100 mL) and the filtrate was concentrated in vacuo. The crude material was purified by combi-flash chromatography, by eluting with 15% MeOH in DCM, to afford tert-butyl (R)-6-(6-(1-(tert-butoxycarbonyl)piperidin-4-yl)nicotinamido)-2-(1-methylpyrrolidin-2-yl)-1H-pyrrolo[3,2-c]pyridine-1-carboxylate (Intermediate 505, 0.60 g, 41%) as an off-white solid. ¹H NMR (400 MHz, DMSO-d₆) δ ppm 1.44 (s, 9H), 1.54-1.88 (m, 16H), 2.35-2.51 (m, 4H), 2.80-2.89 (m, 4H), 3.12-3.16 (m, 1H), 3.92-3.95 (m, 1H), 4.69-4.13 (m, 2H), 6.77 (s, 1H), 7.44 (d, J=8.0 Hz, 1H), 8.31 (d, J=8.0 Hz, 1H), 8.61 (s, 1H), 8.94 (s, 1H), 9.10 (s, 1H), 10.93 (s, 1H). Mass spec m/z 605.3 [M+H]⁺.

### Step 6

### Example 119: 6-(1-((1-(2-(2,6-dioxopiperidin-3-yl)-1-oxoisoindolin-4-yl)piperidin-4-yl)methyl)piperidin-4-yl)-N-(2-((R)-1-methylpyrrolidin-2-yl)-1H-pyrrolo[3,2-c]pyridin-6-yl)nicotinamide

To a solution of (R)-6-(6-(1-(tert-butoxycarbonyl)piperidin-4-yl)nicotinamido)-2-(1-methylpyrrolidin-2-yl)-1H-pyrrolo[3,2-c]pyridine-1-carboxylate (Intermediate 505, 0.50 g, 0.56 mmol) and 3-(4-(4-(dimethoxymethyl)piperidin-1-yl)-1-oxoisoindolin-2-yl)piperidine-2,6-dione (Intermediate 499, 0.33 g, 0.68 mmol) in dimethylformamide (3.0 mL) was added formic acid (5 mL) and the reaction mixture was heated at 60° C. for 3 h. Sodium cyanoborohydride (0.11 g, 1.70 mmol) and triethylamine (0.31 mL, 2.27 mmol) were then added at room temperature and the reaction mixture was heated at 60° C. for 16 h. The reaction mixture was concentrated in vacuo. The crude material was purified by preparative HPLC (Method A) to afford 6-(1-((1-(2-(2,6-dioxopiperidin-3-yl)-1-oxoisoindolin-4-yl)piperidin-4-yl)methyl)piperidin-4-yl)-N-(2-((R)-1-methylpyrrolidin-2-yl)-1H-pyrrolo[3,2-c]pyridin-6-yl)nicotinamide (Example 119, 0.21 g, 51%) as an off-white solid. ¹H NMR (400 MHz, DMSO-d₆) 1.21-1.31 (m, 2H), 1.64-1.93 (m, 10H), 1.96-2.06 (m, 3H), 2.13-2.2.18 (m, 4H), 2.21-2.29 (m, 3H), 2.53-2.61 (m, 1H), 2.71-2.79 (m, 3H), 2.86-3.12 (m, 3H), 3.13-3.18 (m, 1H), 3.39-3.42 (m, 4H), 4.27-4.31 (m, 1H), 4.42-4.46 (m, 1H), 5.10-5.14 (m, 1H), 6.40 (s, 1H), 7.16 (d, J=7.6 Hz, 2H), 7.29 (d, J=7.2 Hz, 1H), 7.40-7.45 (m, 1H), 8.20 (s, 1H), 8.29-8.31 (m, 1H), 8.51 (s, 1H), 9.09 (s, 1H), 10.69 (s, 1H), 10.99 (s, 1H), 11.39 (s, 1H). Mass spec m/z 744.3 [M+H]⁺.

### Example 120 was Synthesised Following Scheme 118

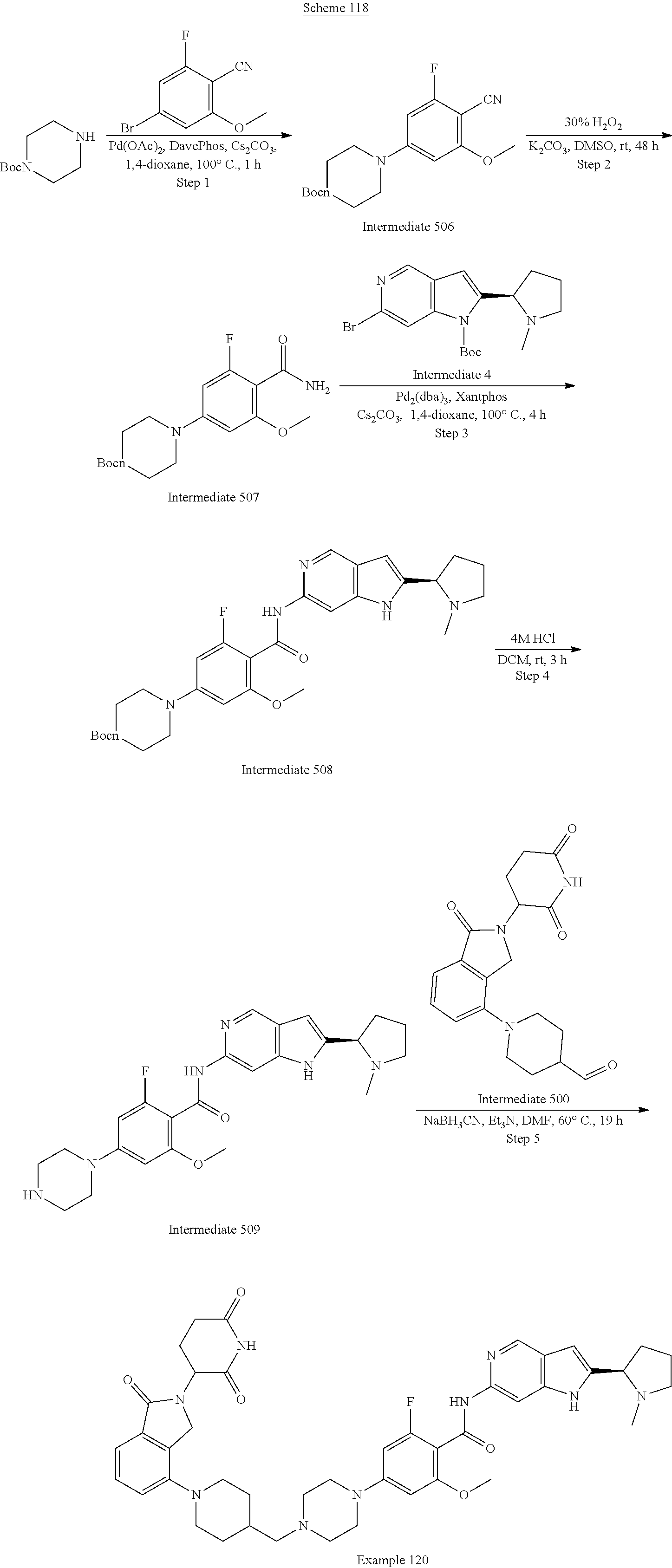

### Step 1

### Intermediate 506: tert-butyl 4-(4-cyano-3-fluoro-5-methoxyphenyl)piperazine-1-carboxylate

To a solution of tert-butyl piperazine-1-carboxylate (CAS No. 57260-71-6, 4.90 g, 26.0 mmol) in 1,4-dioxane (30 mL) was added 4-bromo-2-fluoro-6-methoxybenzonitrile (CAS No. 457051-15-9, 5.60 g, 24.0 mmol) followed by cesium carbonate (24.0 g, 72.0 mmol). The reaction mixture was purged with argon for 15 min then DavePhos (1.50 g, 3.60 mmol) and palladium (II) acetate (0.56 g, 2.40 mmol) were added. The reaction mixture was further purged with argon for 10 min and heated at 100° C. for 1 h. The reaction mixture was concentrated in vacuo and the crude material was purified by combi-flash chromatography, by eluting with 20% ethyl acetate in heptane, to afford tert-butyl 4-(4-cyano-3-fluoro-5-methoxyphenyl)piperazine-1-carboxylate (Intermediate 506, 6.0 g, 73%) as a yellow solid. ¹H NMR (400 MHz, DMSO-d₆) δ ppm 1.42 (s, 9H), 3.40-3.49 (m, 8H), 3.89 (s, 3H), 6.37 (s, 1H), 6.54 (d, J=13.60 Hz, 1H). Mass spec m/z 336.2 [M+H]⁺.

### Step 2

### Intermediate 507: tert-butyl 4-(4-carbamoyl-3-fluoro-5-methoxyphenyl)piperazine-1-carboxylate

To a solution of tert-butyl 4-(4-cyano-3-fluoro-5-methoxyphenyl)piperazine-1-carboxylate (Intermediate 506, 2.0 g, 5.85 mmol) in dimethyl sulfoxide (15 mL) was added potassium carbonate (1.20 g, 8.69 mmol) followed by 30% hydrogen peroxide (4.75 mL, 58.5 mmol) and the reaction mixture was stirred at room temperature for 48 h. The reaction mixture was quenched with ice cold water (100 mL), the resultant precipitate was collected by filtration and dried in vacuo to afford tert-butyl 4-(4-carbamoyl-3-fluoro-5-methoxyphenyl)piperazine-1-carboxylate (Intermediate 507, 0.9 g, 17%) as a white solid which was used for next step without further purification. Mass spec m/z 354.2 [M+H]⁺.

### Step 3

### Intermediate 508: tert-butyl (R)-6-(4-(4-(tert-butoxycarbonyl)piperazin-1-yl)-2-fluoro-6-methoxybenzamido)-2-(1-methylpyrrolidin-2-yl)-1H-pyrrolo[3,2-c]pyridine-1-carboxylate

To a solution of tert-butyl 4-(4-carbamoyl-3-fluoro-5-methoxyphenyl)piperazine-1-carboxylate (Intermediate 507, 0.60 g, 1.69 mmol) in 1,4-dioxane (30 mL) was added tert-butyl (R)-6-bromo-2-(1-methylpyrrolidin-2-yl)-1H-pyrrolo[3,2-c]pyridine-1-carboxylate (Intermediate 4, 0.71 g, 1.86 mmol) followed by cesium carbonate (1.66 g, 5.09 mmol). The reaction mixture was purged with argon for 15 min, then Xantphos (0.30 g, 0.50 mmol) and Pd₂(dba)₃ (0.20 g, 0.25 mmol) were added. The reaction mixture was further purged with argon for 10 min and heated at 100° C. for 4 h. The reaction mixture was concentrated in vacuo and the crude material was purified by combi-flash chromatography, by eluting with 12% MeOH in DCM, to afford tert-butyl (R)-6-(4-(4-(tert-butoxycarbonyl)piperazin-1-yl)-2-fluoro-6-methoxybenzamido)-2-(1-methylpyrrolidin-2-yl)-1H-pyrrolo[3,2-c]pyridine-1-carboxylate (Intermediate 508, 0.8 g, 17%) as an off-white solid. ¹H NMR (400 MHz, DMSO-d₆) δ ppm 1.42 (s, 9H), 1.55-1.59 (m, 1H), 1.68 (s, 9H), 1.70-1.78 (m, 3H), 2.27 (s, 3H), 2.32-2.36 (m, 2H), 3.21-3.26 (m, 4H), 3.43-3.50 (m, 4H), 3.79 (s, 3H), 3.91-3.93 (m, 1H), 6.38 (s, 1H), 6.40 (s, 1H), 6.71 (s, 1H), 8.48 (s, 1H), 8.90 (s, 1H), 10.54 (br s, 1H).

### Step 4

### Intermediate 509: (R)-2-fluoro-6-methoxy-N-(2-(1-methylpyrrolidin-2-yl)-1H-pyrrolo[3,2-c]pyridin-6-yl)-4-(piperazin-1-yl)benzamide dihydrochloride

To a cooled (0° C.) solution of tert-butyl (R)-6-(4-(4-(tert-butoxycarbonyl)piperazin-1-yl)-2-fluoro-6-methoxybenzamido)-2-(1-methylpyrrolidin-2-yl)-1H-pyrrolo[3,2-c]pyridine-1-carboxylate (Intermediate 508, 0.50 g, 0.76 mmol) in dichloromethane (15 mL) was added 4M hydrochloric acid in 1,4-dioxane (10 mL) and the reaction mixture was stirred at room temperature for 3 h. The reaction mixture was concentrated in vacuo to afford (R)-2-fluoro-6-methoxy-N-(2-(1-methylpyrrolidin-2-yl)-1H-pyrrolo[3,2-c]pyridin-6-yl)-4-(piperazin-1-yl)benzamide dihydrochloride (Intermediate 509, 0.45 g, 98%) as a brown solid, which was used for the next step without further purification. Mass spec m/z 453.1 [M+H]⁺.

### Step 5

### Example 120: 4-(4-((1-(2-(2,6-dioxopiperidin-3-yl)-1-oxoisoindolin-4-yl)piperidin-4-yl)methyl)piperazin-1-yl)-2-fluoro-6-methoxy-N-(2-((R)-1-methylpyrrolidin-2-yl)-1H-pyrrolo[3,2-c]pyridin-6-yl)benzamide

To a solution of (R)-2-fluoro-6-methoxy-N-(2-(1-methylpyrrolidin-2-yl)-1H-pyrrolo[3,2-c]pyridin-6-yl)-4-(piperazin-1-yl)benzamide hydrochloride (Intermediate 509, 0.40 g, 0.88 mmol) in N,N dimethylformamide (15 mL) was added 1-(2-(2,6-dioxopiperidin-3-yl)-1-oxoisoindolin-4-yl)piperidine-4-carbaldehyde (Intermediate 500, 0.37 g, 0.88 mmol) followed by triethylamine (0.49 mL, 3.53 mmol) and the reaction mixture was heated at 60° C. for 3 h. Sodium cyanoborohydride (0.17 g, 2.65 mmol) was then added and the reaction mixture was heated at 60° C. for 16 h. The reaction mixture was diluted with ice cold water (80 mL), the resultant precipitate was collected by filtration and dried in vacuo. The crude material was purified by preparative HPLC (Method A) to afford 4-(4-((1-(2-(2,6-dioxopiperidin-3-yl)-1-oxoisoindolin-4-yl)piperidin-4-yl)methyl)piperazin-1-yl)-2-fluoro-6-methoxy-N-(2-((R)-1-methylpyrrolidin-2-yl)-1H-pyrrolo[3,2-c]pyridin-6-yl)benzamide (Example 120, 0.10 g, 14%) as an off-white solid. ¹H NMR (400 MHz, DMSO-d₆) δ ppm 1.24-1.33 (m, 2H), 1.77-1.92 (m, 6H), 1.99-2.05 (m, 1H), 2.11-2.14 (m, 1H), 2.15 (s, 3H), 2.22-2.30 (m, 3H), 2.41-2.46 (m, 2H), 2.53-2.57 (m, 3H), 2.71-2.76 (m, 1H), 2.88-2.96 (m, 2H), 3.12-3.15 (m, 1H), 3.26-3.31 (m, 5H), 3.36-3.42 (m, 2H), 3.80 (s, 3H), 4.27-4.31 (m, 2H), 4.41-4.46 (m, 2H), 5.09-5.14 (m, 1H), 6.36 (s, 1H), 6.38 (s, 1H), 7.16 (d, J=8.21 Hz, 1H), 7.70 (d, J=8.21 Hz, 1H), 7.43 (t, J=7.6 Hz, 1H), 8.17 (s, 1H), 8.41 (s, 1H), 10.20 (br s, 1H), 10.98 (br s, 1H), 11.31 (br s, 1H). Mass spec m/z 792.3 [M+H]⁺.

### Example 121 was Synthesised Following Scheme 119

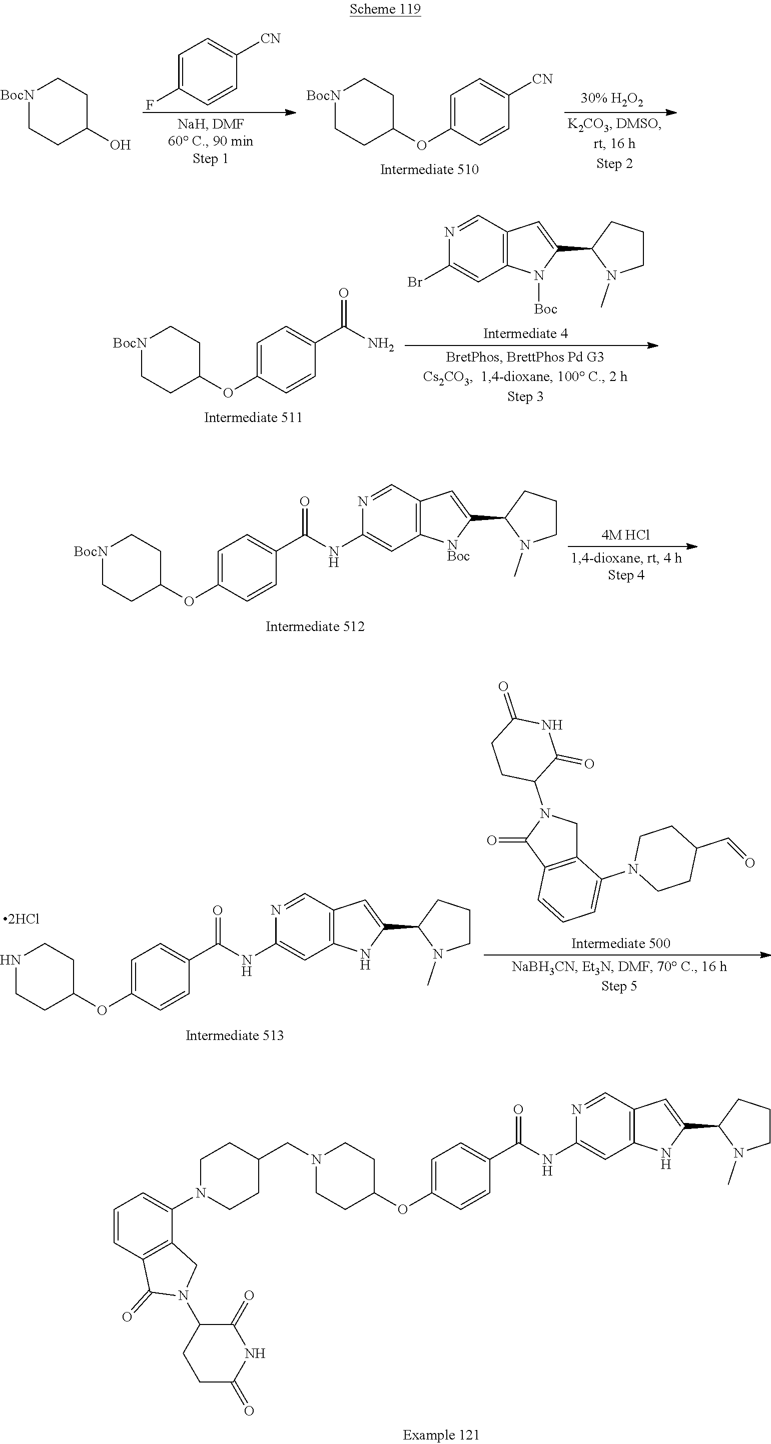

### Step 1

### Intermediate 510: tert-butyl 4-(4-cyanophenoxy) piperidine-1-carboxylate

To a solution of tert-butyl 4-hydroxypiperidine-1-carboxylate (CAS No: 109384-19-2, 20.0 g, 99.37 mmol) in dimethylformamide (30 mL) was added 60% sodium hydride (7.94 g, 198.7 mmol) and the reaction mixture was stirred at room temperature for 1 h. 4-Fluorobenzonitrile (CAS No: 1194-02-1, 13.24 g, 109.3 mmol) was then added and the reaction mixture was heated at 60° C. for 30 min. After completion, the reaction mixture was quenched with ice cold water (200 mL), the resultant precipitate was collected by filtration and dried in vacuo. The crude material was purified by combi-flash chromatography, by eluting with 20% ethyl acetate in heptane, to afford tert-butyl 4-(4-cyanophenoxy)piperidine-1-carboxylate (Intermediate 510, 25.0 g, 83%) as a white solid. ¹H NMR (400 MHz, DMSO-d₆) δ ppm 1.40 (s, 9H), 1.51-1.53 (m, 2H), 1.89-1.93 (m, 2H), 3.10-3.22 (m, 2H), 3.62-3.69 (m, 2H), 4.67-4.74 (m, 1H), 7.14 (d, J=8.61 Hz, 2H), 7.75 (d, J=8.61 Hz, 2H). Mass spec m/z 247.17 [M−56]-Step 2

### Intermediate 511: tert-butyl 4-(4-carbamoylphenoxy)piperidine-1-carboxylate

To a solution of tert-butyl 4-(4-cyanophenoxy)piperidine-1-carboxylate (Intermediate 510, 6.0 g, 19.8 mmol) in dimethyl sulfoxide (10 mL) was added potassium carbonate (4.11 g, 29.8 mmol) and hydrogen peroxide (5.12 mL, 59.5 mmol) and the reaction mixture was stirred at room temperature for 16 h. After completion, the reaction mixture was quenched with ice cold water (200 mL), the resultant precipitate was collected by filtration and dried in vacuo to afford tert-butyl 4-(4-carbamoylphenoxy)piperidine-1-carboxylate (Intermediate 511, 5.0 g, 79%) as a white solid, which was used in the next step with further purification. ¹H NMR (400 MHz, DMSO-d₆) δ ppm 1.41 (s, 9H), 1.51-1.53 (m, 2H), 1.89-1.93 (m, 2H), 3.14-3.26 (m, 2H), 3.61-3.62 (m, 2H), 4.61-4.69 (m, 1H), 7.01 (d, J=8.22 Hz, 2H), 7.15 (br s, 1H), 7.79-7.83 (m, 2H), 7.84 (br s, 1H). Mass spec m/z 319.0 [M−H]⁻.

### Step 3

### Intermediate 512: tert-butyl (R)-6-(4-((1-(tert-butoxycarbonyl)piperidin-4-yl)oxy)benzamido)-2-(1-methylpyrrolidin-2-yl)-1H-pyrrolo[3,2-c]pyridine-1-carboxylate

To a solution of tert-butyl 4-(4-carbamoylphenoxy)piperidine-1-carboxylate (Intermediate 511, 2.0 g, 6.24 mmol) and tert-butyl (R)-6-bromo-2-(1-methylpyrrolidin-2-yl)-1H-pyrrolo[3,2-c]pyridine-1-carboxylate (Intermediate 4, 2.85 g, 7.49 mmol) in 1,4-dioxane (20 mL) was added cesium carbonate (6.11 g, 18.17 mmol). The reaction mixture was purged with argon for 15 min, then BrettPhos Pd G3 (0.86 g 0.93 mmol) and BrettPhos (0.51 g, 0.93 mmol) were added. The reaction mixture was further purged with argon for 10 min and heated at 100° C. for 2 h. After completion, the reaction mixture was concentrated in vacuo and the crude material was purified by combi-flash chromatography, by eluting with 90% ethyl acetate in heptane, to afford tert-butyl (R)-6-(4-((1-(tert-butoxycarbonyl)piperidin-4-yl)oxy)benzamido)-2-(1-methylpyrrolidin-2-yl)-1H-pyrrolo[3,2-c]pyridine-1-carboxylate (Intermediate 512, 2.0 g, 52%) as a brown solid. ¹H NMR (400 MHz, DMSO-d₆) δ ppm 1.41 (s, 9H), 1.49-1.65 (m, 4H), 1.69 (s, 9H), 1.72-1.79 (m, 2H), 1.90-1.97 (m, 2H), 2.28-2.33 (m, 1H), 2.35 (s, 3H), 2.38-2.44 (m, 1H), 3.10-3.26 (m, 3H), 3.62-3.75 (m, 2H), 3.88-3.96 (m, 1H), 6.75 (s, 1H), 7.06 (d, J=6.65 Hz, 2H), 8.04 (d, J=6.65 Hz, 2H), 8.58 (s, 1H), 8.92 (s, 1H), 10.51 (br s, 1H). Mass spec m/z 620.3 [M+H]⁺.

### Step 4

### Intermediate 513: (R)—N-(2-(1-methylpyrrolidin-2-yl)-1H-pyrrolo[3,2-c]pyridin-6-yl)-4-(piperidin-4-yloxy)benzamide dihydrochloride

To a cooled (0° C.) solution of tert-butyl (R)-6-(4-((1-(tert-butoxycarbonyl)piperidin-4-yl)oxy)benzamido)-2-(1-methylpyrrolidin-2-yl)-1H-pyrrolo[3,2-c]pyridine-1-carboxylate (Intermediate 512, 2.0 g, 3.22 mmol) in 1,4-dioxane (10 mL) was added 4M hydrochloric acid in 1,4-dioxane (10 mL, 40.0 mmol) and the reaction mixture was stirred at room temperature for 4 h. After completion, the reaction mixture was concentrated in vacuo to afford (R)—N-(2-(1-methylpyrrolidin-2-yl)-1H-pyrrolo[3,2-c]pyridin-6-yl)-4-(piperidin-4-yloxy)benzamide dihydrochloride (Intermediate 513, 1.7 g) as a yellow semi-solid, which was used for the next step without further purification. ¹H NMR (400 MHz, DMSO-d₆) δ ppm 1.56-1.62 (m, 2H), 1.88-1.91 (m, 2H), 2.14-2.20 (m, 4H), 2.37-2.42 (m, 2H), 2.55-2.57 (m, 1H), 3.05-3.13 (m, 2H), 3.21-3.30 (m, 3H), 3.32-3.34 (m, 2H), 3.70-3.80 (m, 1H), 7.21 (d, J=9.07 Hz, 2H), 7.28 (s, 1H), 8.23 (d, J=8.69 Hz, 2H), 8.29 (s, 1H), 9.08 (s, 2H), 11.52 (br s, 1H), 11.98 (br s, 1H), 13.31 (br s, 1H). Mass spec m/z 420.2 [M+H]⁺.

### Step 5

### Example 121: 4-((1-((1-(2-(2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide

To a solution of (R)—N-(2-(1-methylpyrrolidin-2-yl)-1H-pyrrolo[3,2-c]pyridin-6-yl)-4-(piperidin-4-yloxy)benzamide dihydrochloride (Intermediate 513, 0.59 g, 1.31 mmol) in dimethylformamide (10 mL) was added triethylamine (0.91 mL, 6.55 mmol) followed by 1-(2-(2,6-dioxopiperidin-3-yl)-1-oxoisoindolin-4-yl)piperidine-4-carbaldehyde 2,2,2-trifluoroacetate (Intermediate 500, 0.61 g, 1.31 mmol) and the reaction mixture was heated at 70° C. for 1 h. Sodium cyanoborohydride (0.34 g 5.24 mmol) was then added and reaction mixture was heated at 70° C. for 16 h. After completion, the reaction mixture was quenched with ice cold water (100 mL), the resultant precipitate was collected by filtration and dried in vacuo. The crude material was purified by preparative HPLC (Method A) to afford 4-((1-((1-(2-(2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide (Example 121, 0.20 g, 20%) as an as off-white solid. ¹H NMR (400 MHz, DMSO-d₆) δ ppm 1.24-1.27 (m, 2H), 1.63-1.67 (m, 3H), 1.81-2.00 (m, 8H), 2.13-2.33 (m, 9H), 2.37-2.46 (m, 1H), 2.52-2.63 (m, 2H), 2.65-2.79 (m, 4H), 2.85-2.98 (m, 1H), 3.08-3.21 (m, 1H), 3.33-3.43 (m, 2H), 4.26-4.31 (m, 1H), 4.41-4.45 (m, 1H), 4.48-4.55 (m, 1H), 5.09-5.14 (m, 1H), 6.38 (s, 1H), 7.03 (d, J=8.88 Hz, 2H), 7.15 (d, J=7.75 Hz, 1H), 7.29 (d, J=7.25 Hz, 1H), 7.42 (t, J=7.69 Hz, 1H), 8.02 (d, J=8.88 Hz, 2H), 8.17 (s, 1H), 8.48 (s, 1H), 10.25 (br s, 1H), 10.97 (br s, 1H), 11.33 (br s, 1H). Mass spec m/z 759.43 [M+H]⁺.

### Examples 122 and 123 were prepared following Scheme 120

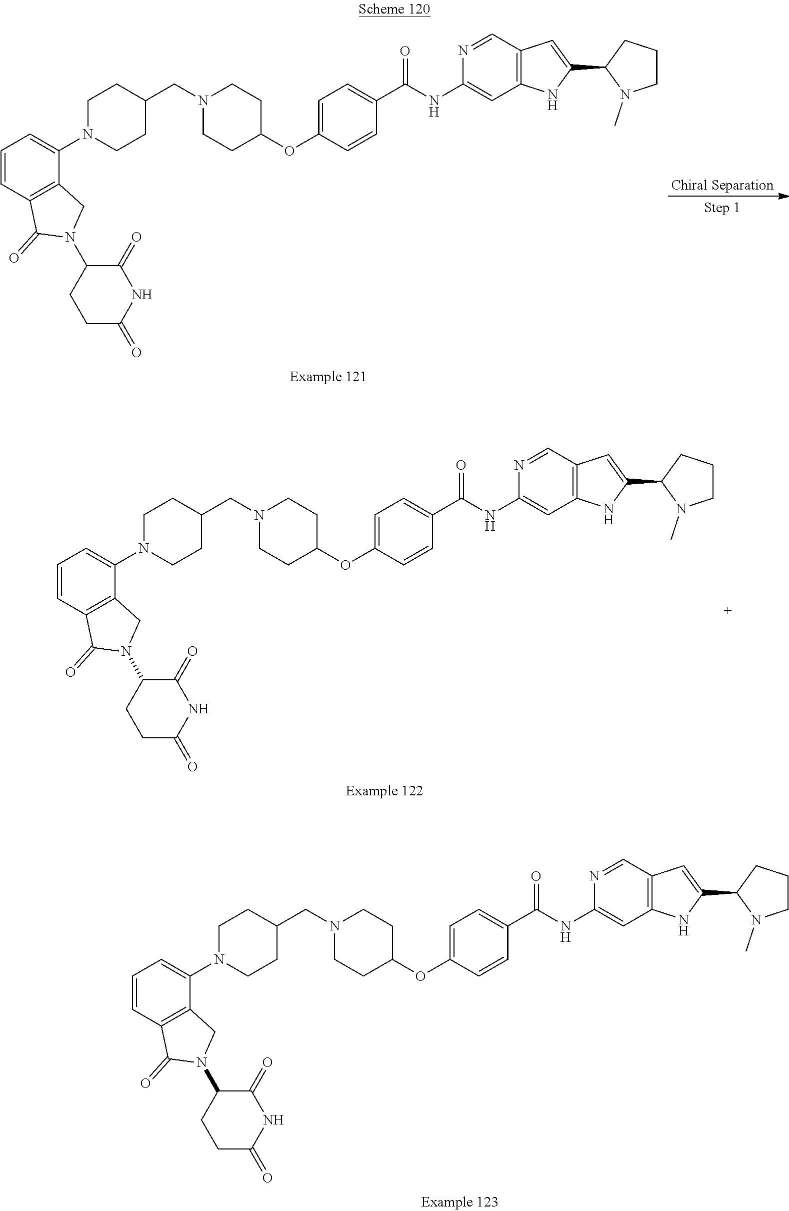

### Step 1

### Example 122: 4-((1-((1-(2-((rel-S)-2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide and Example 123: 4-((1-((1-(2-((rel-R)-2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide

4-((1-((1-(2-(2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide (Example 121, 0.15 g, 0.19 mmol) was separated by chiral preparative HPLC (Column: R,R WHELK (30×250 mm), 5 m; Mobile Phase A: DCM, Mobile Phase B: IPA; Flow rate: 40 ml/min; isocratic 30% B) to afford 4-((1-((1-(2-((rel-S)-2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide (Example 122, 1st eluting peak, 0.05 g, 33%) as an off-white solid and 4-((1-((1-(2-((rel-R)-2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide (Example 123, 2nd eluting peak, 0.026 g, 17%) as an off-white solid

### Example 122: 4-((1-((1-(2-((rel-S)-2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide

1st eluting peak from chiral preparative HPLC. Retention time: 4.19 mins.

¹H NMR (400 MHz, DMSO-d6) δ ppm 1.23-1.27 (m, 2H), 1.60-1.73 (m, 3H), 1.81-1.92 (m, 5H), 1.97-2.00 (m, 3H), 2.11-2.15 (m, 1H), 2.17 (s, 3H), 2.18-2.31 (m, 5H), 2.41-2.45 (m, 2H), 2.53-2.64 (m, 1H), 2.65-2.77 (m, 4H), 2.86-2.98 (m, 1H), 3.10-3.18 (m, 1H), 3.35-3.43 (m, 2H), 4.26-4.31 (m, 1H), 4.38-4.45 (m, 1H), 4.50-4.56 (m, 1H), 5.09-5.13 (m, 1H), 6.38 (s, 1H), 7.03 (d, J=9.01 Hz, 2H), 7.16 (d, J=7.63 Hz, 1H), 7.29 (d, J=7.00 Hz, 1H), 7.40-7.46 (m, 1H), 8.02 (d, J=8.88 Hz, 2H), 8.17 (s, 1H), 8.48 (s, 1H), 10.25 (br s, 1H), 10.97 (br s, 1H), 11.33 (br s, 1H). Mass spec m/z 759.3 [M+H]⁺.

### Example 123: 4-((1-((1-(2-((rel-R)-2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide

2nd eluting peak from chiral preparative HPLC. Retention time: 4.91 mins.

¹H NMR (400 MHz, DMSO-d6) δ ppm 1.23-1.32 (m, 2H), 1.64-1.69 (m, 3H), 1.75-1.93 (m, 5H), 1.93-2.04 (m, 3H), 2.11-2.15 (m, 1H), 2.16 (s, 3H), 2.20-2.30 (m, 5H), 2.53-2.64 (m, 3H), 2.66-2.74 (m, 4H), 2.86-2.99 (m, 1H), 3.12-3.16 (m, 1H), 3.37-3.44 (m, 2H), 4.26-4.33 (m, 1H), 4.30-4.45 (m, 1H), 4.48-4.57 (m, 1H), 5.09-5.13 (m, 1H), 6.38 (s, 1H), 7.03 (d, J=8.80 Hz, 2H), 7.16 (d, J=7.82 Hz, 1H), 7.29 (d, J=7.34 Hz, 1H), 7.39-7.42 (m, 1H), 8.02 (d, J=8.80 Hz, 2H), 8.17 (s, 1H), 8.48 (s, 1H), 10.27 (br s, 1H), 10.99 (br s, 1H), 11.34 (br s, 1H). Mass spec m/z 759.3 [M+H]⁺.

### Example 124 was Prepared Following Scheme 121

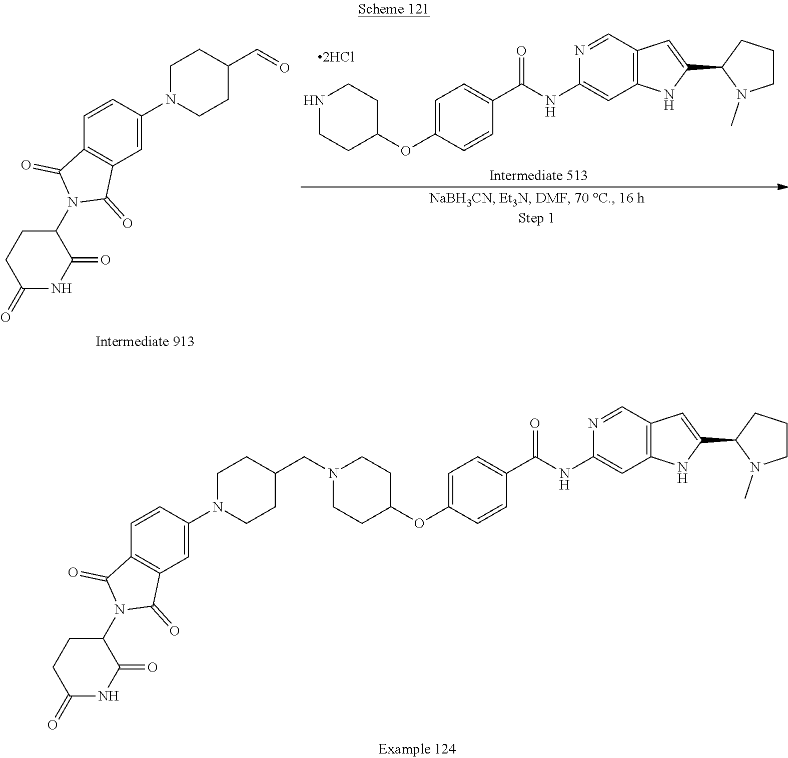 [SMILES: O=CC1CCN(c2ccc3c(c2)C(=O)N(C2CCC(=O)NC2=O)C3=O)CC1 ; CN1CCC[C@@H]1c1cc2cnc(NC(=O)c3ccc(OC4CCNCC4)cc3)cc2[nH]1 ; CN1CCC[C@@H]1c1cc2cnc(NC(=O)c3ccc(OC4CCN(CC5CCN(c6ccc7c(c6)C(=O)N(C6CCC(=O)NC6=O)C7=O)CC5)CC4)cc3)cc2[nH]1]

### Step 1

### Example 124: 4-((1-((1-(2-(2,6-dioxopiperidin-3-yl)-1,3-dioxoisoindolin-5-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide

To a solution of 1-(2-(2,6-dioxopiperidin-3-yl)-1,3-dioxoisoindolin-5-yl)piperidine-4-carbaldehyde (Intermediate 913, 0.8 g, 1.65 mmol) in dimethylformamide (10 mL) was added triethylamine (1.16 mL, 8.27 mmol) followed by (R)—N-(2-(1-methylpyrrolidin-2-yl)-1H-pyrrolo[3,2-c]pyridin-6-yl)-4-(piperidin-4-yloxy)benzamide dihydrochloride (Intermediate 513, 0.75 g, 1.65 mmol) and the reaction mixture was heated at 70° C. for 16 h. Sodium cyanoborohydride (0.43 g 6.62 mmol) was then added and the reaction mixture was heated at 70° C. for 16 h. After completion, the reaction mixture was quenched with ice cold water (100 mL), the resultant precipitate was collected by filtration and dried in vacuo. The crude material was purified by preparative HPLC (Method A) to afford 4-((1-((1-(2-(2,6-dioxopiperidin-3-yl)-1,3-dioxoisoindolin-5-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide (Example 124, 0.17 g, 14%) as a yellow solid. ¹H NMR (400 MHz, DMSO-d₆) δ ppm 1.13-1.17 (m, 2H), 1.64-1.69 (m, 2H), 1.74-1.92 (m, 6H), 1.94-2.06 (m, 3H), 2.13-2.15 (m, 1H), 2.17 (s, 3H), 2.17-2.20 (m, 2H), 2.20-2.31 (m, 3H), 2.53-2.59 (m, 1H), 2.64-2.75 (m, 2H), 2.82-2.90 (m, 1H), 2.91-3.02 (m, 2H), 3.10-3.18 (m, 1H), 3.27-3.30 (m, 2H), 4.02-4.06 (m, 2H), 4.47-4.56 (m, 1H), 5.03-5.08 (m, 1H), 6.38 (s, 1H), 7.03 (d, J=8.88 Hz, 2H), 7.23 (dd, J=8.69, 2.19 Hz, 1H), 7.31 (s, 1H), 7.65 (d, J=8.50 Hz, 1H), 8.02 (d, J=8.88 Hz, 2H), 8.17 (s, 1H), 8.48 (s, 1H), 10.25 (s, 1H), 11.06 (s, 1H), 11.33 (s, 1H). Mass spec m/z 773.0 [M+H]⁺.

### Example 125 was Prepared Following Scheme 122

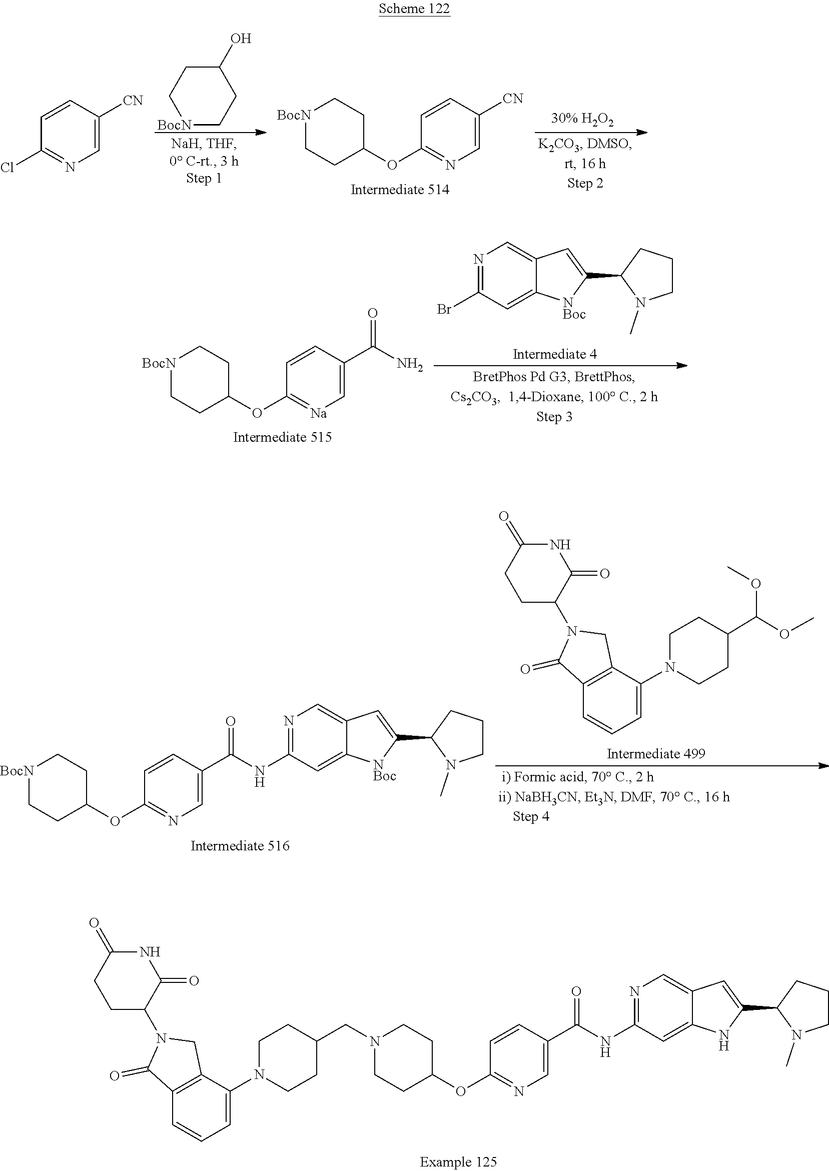

### Step 1

### Intermediate 514: tert-butyl 4-((5-cyanopyridin-2-yl)oxy)piperidine-1-carboxylate

To a cooled (0° C.) solution of tert-butyl 4-hydroxypiperidine-1-carboxylate (CAS No. 109384-19-2, 5 g, 24.843 mmol) and 6-chloro-3-pyridinecarbonitrile (CAS No. 33252-28-7, 3.79 g, 27.33 mmol) in tetrahydrofuran (100 mL) was added sodium hydride (60% in mineral oil, 1.49 g, 37.26 mmol) and the reaction mixture was stirred for 3 h at room temperature. The reaction mixture was then quenched with water (100 mL) and extracted with ethyl acetate (2×100 mL). The combined organic layers were dried over Na₂SO₄, filtered and concentrated in vacuo to afford tert-butyl 4-((5-cyanopyridin-2-yl)oxy)piperidine-1-carboxylate (Intermediate 514, 6.2 g, 82%) as an off-white solid, which was used for the next step without further purification. ¹H NMR (400 MHz, DMSO-d₆) δ ppm 1.40 (s, 9H), 1.52-1.58 (m, 2H), 1.91-1.97 (m, 2H), 3.16-3.19 (m, 2H), 3.65-3.72 (m, 2H), 5.23-5.27 (m, 1H), 6.98 (d, J=8.80 Hz, 1H), 8.14 (d, J=8.80 Hz, 1H), 8.68 (s, 1H). Mass spec m/z 248.1 [M+H]⁺.

### Step 2

### Intermediate 515: tert-butyl 4-((5-cyanopyridin-2-yl)oxy)piperidine-1-carboxylate

To a solution of tert-butyl 4-((5-cyanopyridin-2-yl)oxy)piperidine-1-carboxylate (Intermediate 514, 6.2 g, 20.0 mmol) in dimethyl sulfoxide (10 mL) was added potassium carbonate (5.6 g, 41.0 mmol) followed by 30% H₂O₂ (6.67 ml, 61.0 mmol). The reaction mixture was stirred at room temperature for 16 h, then quenched with ice cold water (100 mL). The resultant precipitate was collected by filtration and dried in vacuo to afford tert-butyl 4-((5-cyanopyridin-2-yl)oxy)piperidine-1-carboxylate (Intermediate 515, 6.0 g, 91%) as an off-white solid, which was used for the next step without further purification. ¹H NMR (400 MHz, DMSO-d₆) δ ppm 1.41 (s, 9H), 1.52-1.58 (m, 2H), 1.92-1.97 (m, 2H), 3.14-3.20 (m, 2H), 3.67-3.72 (m, 2H), 5.22-5.26 (m, 1H), 6.84 (d, J=8.80 Hz, 1H), 7.39 (s, 1H), 7.96 (s, 1H), 8.12 (d, J=8.80 Hz, 1H), 8.66 (s, 1H). Mass spec m/z 322.0 [M+H]⁺.

### Step 3

### Intermediate 516: tert-butyl (R)-6-(6-((1-(tert-butoxycarbonyl)piperidin-4-yl)oxy)nicotinamido)-2-(1-methylpyrrolidin-2-yl)-1H-pyrrolo[3,2-c]pyridine-1-carboxylate

To a solution of tert-butyl 4-((5-cyanopyridin-2-yl)oxy)piperidine-1-carboxylate (Intermediate 515, 0.53 g, 1.66 mmol) and tert-butyl (R)-6-bromo-2-(1-methylpyrrolidin-2-yl)-1H-pyrrolo[3,2-c]pyridine-1-carboxylate (Intermediate 4, 0.70 g, 1.84 mmol) in 1,4-dioxane (10 mL) was added cesium carbonate (1.80 g, 5.52 mmol). The reaction mixture was purged with argon gas for 15 min, then BrettPhos (0.29 g, 0.56 mmol) and BrettPhos Pd G3 (0.25 g, 0.28 mmol) were added. The reaction mixture was further purged with argon gas for 10 min and then heated at 100° C. for 2 h. The reaction mixture was filtered through a celite bed and the filter pad was washed with ethyl acetate (200 mL). The filtrate was concentrated in vacuo and the crude material was purified by combi-flash chromatography, by eluting with ethyl acetate, to afford tert-butyl (R)-6-(6-((1-(tert-butoxycarbonyl)piperidin-4-yl)oxy)nicotinamido)-2-(1-methylpyrrolidin-2-yl)-1H-pyrrolo[3,2-c]pyridine-1-carboxylate (Intermediate 516, 0.65 g, 57%) as an off-white solid. ¹H NMR (400 MHz, DMSO-d₆) δ ppm 1.41 (s, 9H), 1.52-1.75 (m, 14H), 1.96-2.10 (m, 2H), 2.31-2.41 (m, 5H), 3.11-3.27 (m, 3H), 3.69-3.75 (m, 2H), 3.87-3.39 (m, 1H), 5.26-5.31 (m, 1H), 6.75 (s, 1H), 6.88 (d, J=8.80 Hz, 1H), 8.29 (dd, J=8.80, 2.40 Hz, 1H), 8.59 (s, 1H), 8.82 (d, J=2.00 Hz, 1H), 8.91 (s, 1H), 10.82 (s, 1H). Mass spec m/z 621.5 [M+H]⁺.

### Step 4

### Example 125: 6-((1-((1-(2-(2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)nicotinamide

A solution of 3-(4-(4-(dimethoxymethyl)piperidin-1-yl)-1-oxoisoindolin-2-yl)piperidine-2,6-dione (Intermediate 499, 0.39 g, 0.97 mmol) in formic acid (4 mL) was heated at 70° C. for 2 h. The reaction was cooled to room temperature, then dimethylformamide (3.0 mL), (R)-6-(6-((1-(tert-butoxycarbonyl)piperidin-4-yl)oxy)nicotinamido)-2-(1-methylpyrrolidin-2-yl)-1H-pyrrolo[3,2-c]pyridine-1-carboxylate (Intermediate 516, 0.60 g, 0.56 mmol), sodium cyanoborohydride (0.12 g, 1.93 mmol) and triethylamine (0.54 mL, 3.88 mmol) were added. The reaction mixture was heated at 70° C. for 16 h, then concentrated in vacuo. The crude material was purified by preparative HPLC (Method A) to afford 6-((1-((1-(2-(2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)nicotinamide (Example 125, 0.010 g, 1.4%) as an off-white solid. ¹H NMR (400 MHz, DMSO-d₆) δ ppm 1.12-1.31 (m, 2H), 1.65-1.71 (m, 3H), 1.75-1.92 (m, 6H), 1.95-2.02 (m, 3H), 2.13-2.30 (m, 11H), 2.53-2.60 (m, 1H), 2.67-2.78 (m, 4H), 2.88-2.95 (m, 1H), 3.13-3.16 (m, 2H), 4.26-4.30 (m, 1H), 4.40-4.45 (m, 1H), 5.09-5.14 (m, 2H), 6.69 (s, 1H), 6.86 (d, J=8.80 Hz, 1H), 7.15 (s, 1H), 7.29 (d, J=7.20 Hz, 1H), 7.42 (t, J=8.00 Hz, 1H), 8.11-8.19 (m, 1H), 8.28 (dd, J=8.80, 2.40 Hz, 1H), 8.50 (s, 1H), 8.82 (d, J=2.40 Hz, 1H), 10.58 (s, 1H), 11.00 (s, 1H), 11.39 (s, 1H). Mass spec m/z 740.6 [M+H]⁺.

¹H NMR (400 MHz, DMSO-d₆) δ ppm 1.76-1.96 (m, 5H), 1.97-2.06 (m, 1H), 2.11-2.15 (m, 1H), 2.17 (s, 3H), 2.21-2.30 (m, 1H), 2.34-2.42 (m, 1H), 2.44-2.47 (m, 1H), 2.55-2.64 (m, 2H), 2.81-2.88 (m, 2H), 2.89-2.97 (m, 1H), 3.11-3.17 (m, 1H), 3.28-3.30 (m, 1H), 4.31-4.38 (m, 1H), 4.44-4.50 (m, 1H), 5.09-5.12 (m, 1H), 6.39 (s, 1H), 7.39 (d, J=8.25 Hz, 2H), 7.59-7.63 (m, 1H), 7.65-7.71 (m, 2H), 8.00 (d, J=8.38 Hz, 2H), 8.19 (s, 1H), 8.49 (s, 1H), 10.36 (br s, 1H), 11.00 (br s, 1H), 11.36 (br s, 1H). Mass spec m/z 629.3 [M+H]⁺.

### Example 126 was Prepared Following Scheme 123

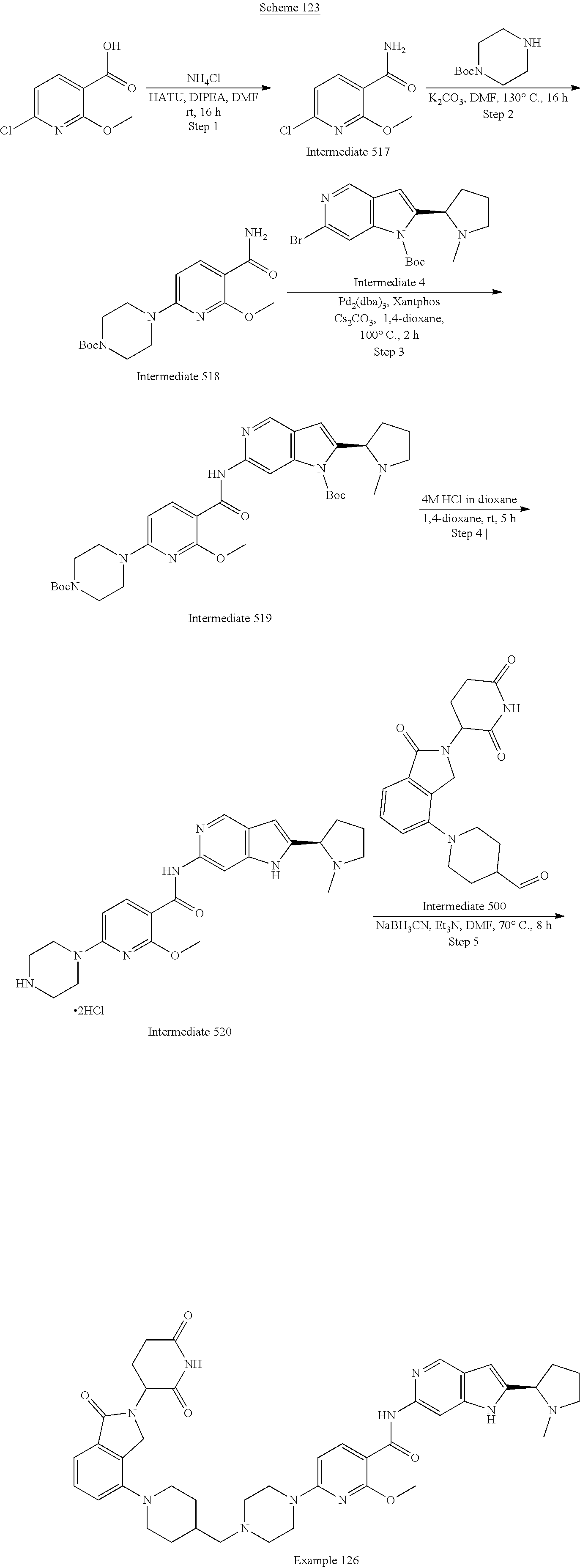

### Step 1

### Intermediate 517: 6-chloro-2-methoxynicotinamide

To a cooled (0° C.) solution of 6-chloro-2-methoxynicotinic acid (CAS No. 65515-33-5, 5.0 g, 26.7 mmol) in dimethylformamide (50 mL) was added HATU (15.67 g, 40.0 mmol) and N,N-diisopropylethylamine (14.0 mL, 80.0 mmol). Ammonium chloride (7.12 g, 133 mmol) was then added, the reaction mixture was stirred at room temperature for 16 h then poured into ice cold water (250 mL). The resultant precipitate was collected by filtration, washed with water (50 mL), and dried in vacuo to afford 6-chloro-2-methoxynicotinamide (Intermediate 517, 4.1 g, 82%) as an off-white solid, which was used for the next step without further purification. ¹H NMR (400 MHz, DMSO-d₆) δ ppm 3.97 (s, 3H), 7.21 (d, J=7.83 Hz, 1H), 7.60-7.80 (m, 2H), 8.17 (d, J=7.83 Hz, 1H). Mass spec m/z 187.0 [M+H]⁺.

### Step 2

### Intermediate 518: tert-butyl 4-(5-carbamoyl-6-methoxypyridin-2-yl)piperazine-1-carboxylate

To a solution of tert-butylpiperazine-1-carboxylate (CAS No. 57260-71-6, 4.1 g, 22.0 mmol) and 6-chloro-2-methoxynicotinamide (Intermediate 517, 4.1 g, 22.0 mmol) in dimethylformamide (40 mL) was added potassium carbonate (9.1 g, 66.0 mmol). The reaction mixture was stirred at 130° C. for 16 h. The reaction mixture was diluted with water (150 mL) and extracted with ethyl acetate (3×200 mL). The combined organic layers were dried over anhydrous Na₂SO₄, filtered and concentrated in vacuo to afford tert-butyl 4-(5-carbamoyl-6-methoxypyridin-2-yl)piperazine-1-carboxylate (Intermediate 518, 3.4 g, 46%) as a white solid, which was used for the next step without further purification. ¹H NMR (400 MHz, DMSO-d₆) δ ppm 1.42 (s, 9H), 3.39-3.47 (m, 4H), 3.56-3.64 (m, 4H), 3.93 (s, 3H), 6.46 (d, J=8.22 Hz, 1H), 7.28 (br s, 1H), 7.35 (br s, 1H), 8.04 (d, J=8.22 Hz, 1H). Mass spec m/z 337.3 [M+H]⁺.

### Step 3

### Intermediate 519: tert-butyl (R)-6-(6-(4-(tert-butoxycarbonyl)piperazin-1-yl)-2-methoxynicotinamido)-2-(1-methylpyrrolidin-2-yl)-1H-pyrrolo[3,2-c]pyridine-1-carboxylate

To a solution of tert-butyl 4-(5-carbamoyl-6-methoxypyridin-2-yl)piperazine-1-carboxylate (Intermediate 518, 1.0 g, 2.97 mmol) in 1,4-dioxane (30 mL) was added tert-butyl (R)-6-bromo-2-(1-methylpyrrolidin-2-yl)-1H-pyrrolo[3,2-c]pyridine-1-carboxylate (Intermediate 4, 1.35 g, 3.56 mmol) followed by cesium carbonate (2.91 g, 8.91 mmol). The reaction mixture was purged with argon for 15 min, then Pd₂(dba)₃ (0.42 g, 0.44 mmol) followed by Xantphos (0.53 g, 0.89 mmol) were added. The reaction mixture was further purged with argon for 10 min and stirred at 100° C. for 2 h. The reaction mixture was filtered through a celite bed, the filter pad was washed with ethyl acetate (100 mL) and the filtrate was concentrated in vacuo. The crude material was purified by combi-flash chromatography, by eluting with 90% ethyl acetate in heptane, to afford tert-butyl (R)-6-(6-(4-(tert-butoxycarbonyl)piperazin-1-yl)-2-methoxynicotinamido)-2-(1-methylpyrrolidin-2-yl)-1H-pyrrolo[3,2-c]pyridine-1-carboxylate (Intermediate 519, 1.8 g, 95%) as a yellow solid. ¹H NMR (400 MHz, DMSO-d₆) δ ppm 1.43 (s, 9H), 1.55-1.67 (m, 2H), 1.70 (s, 9H), 1.73-1.78 (m, 2H), 2.34 (s, 3H), 2.35-2.41 (m, 3H), 3.40-3.49 (m, 4H), 4.06-4.10 (m, 4H), 4.08 (s, 3H), 6.57 (d, J=8.41 Hz, 1H), 6.72 (s, 1H), 7.15-7.44 (m, 2H), 8.19 (d, J=8.41 Hz, 1H), 8.53 (s, 1H). Mass spec m/z 636.3 [M+H]⁺.

### Step 4

### Intermediate 520: (R)-2-methoxy-N-(2-(1-methylpyrrolidin-2-yl)-1H-pyrrolo[3,2-c]pyridin-6-yl)-6-(piperazin-1-yl)nicotinamide hydrochloride

To a cooled (0° C.) solution of tert-butyl (R)-6-(6-(4-(tert-butoxycarbonyl)piperazin-1-yl)-2-methoxynicotinamido)-2-(1-methylpyrrolidin-2-yl)-1H-pyrrolo[3,2-c]pyridine-1-carboxylate (Intermediate 519, 0.5 g, 0.78 mmol) in 1,4-dioxane (5 mL) was added 4M hydrochloric acid in 1,4-dioxane (5 mL). The reaction mixture was stirred at room temperature for 5 h. The reaction mixture was concentrated in vacuo to afford (R)-2-methoxy-N-(2-(1-methylpyrrolidin-2-yl)-1H-pyrrolo[3,2-c]pyridin-6-yl)-6-(piperazin-1-yl)nicotinamide hydrochloride (Intermediate 520, 0.55 g) as a white solid, which was used for the next step without further purification. ¹H NMR (400 MHz, DMSO-d₆) δ ppm 1.06-1.10 (m, 1H), 2.15-2.22 (m, 2H), 2.31-2.42 (m, 1H), 2.86 (s, 3H), 3.20-3.27 (m, 4H), 3.71-3.79 (m, 2H), 3.86-3.93 (m, 4H), 4.08 (s, 3H), 4.69 (d, J=7.46 Hz, 1H), 6.68 (d, J=8.71 Hz, 1H), 7.09 (s, 1H), 8.20 (d, J=8.71 Hz, 1H), 8.33 (s, 1H), 10.44 (br s, 1H), 10.59 (s, 1H), 12.70 (br s, 1H).

### Step 5

### Example 126: 6-(4-((1-(2-(2,6-dioxopiperidin-3-yl)-1-oxoisoindolin-4-yl)piperidin-4-yl)methyl)piperazin-1-yl)-2-methoxy-N-(2-((R)-1-methylpyrrolidin-2-yl)-1H-pyrrolo[3,2-c]pyridin-6-yl) nicotinamide

To a solution of (R)-2-methoxy-N-(2-(1-methylpyrrolidin-2-yl)-1H-pyrrolo[3,2-c]pyridin-6-yl)-6-(piperazin-1-yl)nicotinamide hydrochloride (Intermediate 520, 0.35 g, 0.80 mmol) and 1-(2-(2,6-dioxopiperidin-3-yl)-1-oxoisoindolin-4-yl)piperidine-4-carbaldehyde (Intermediate 500, 0.34 g, 0.96 mmol) in dimethylformamide (10 mL) was added triethylamine (0.40 g, 4.01 mmol). The reaction mixture was stirred at 70° C. for 4 h then cooled to 0° C. prior to the addition of sodium cyanoborohydride (0.17 g, 2.41 mmol). The reaction mixture was heated at 70° C. for 4 h then quenched with ice cold water (100 mL). The resultant precipitate was collected by filtration and dried in vacuo. The crude material was purified by preparative HPLC (Method A) to afford 6-(4-((1-(2-(2,6-dioxopiperidin-3-yl)-1-oxoisoindolin-4-yl)piperidin-4-yl)methyl)piperazin-1-yl)-2-methoxy-N-(2-((R)-1-methylpyrrolidin-2-yl)-1H-pyrrolo[3,2-c]pyridin-6-yl)nicotinamide (Example 126, 0.10 g, 17%) as an off-white solid. ¹H NMR (400 MHz, DMSO-d₆) δ ppm 1.22-1.31 (m, 2H), 1.75-2.00 (m, 8H), 2.09-2.14 (m, 1H), 2.16 (s, 3H), 2.21-2.30 (m, 3H), 2.43-2.47 (m, 2H), 2.53-2.64 (m, 2H), 2.69-2.81 (m, 2H), 2.85-2.98 (m, 1H), 3.08-3.17 (m, 1H), 3.28-3.30 (m, 2H), 3.35-3.45 (m, 2H), 3.62-3.70 (m, 4H), 4.07 (s, 3H), 4.24-4.34 (m, 1H), 4.38-4.48 (m, 1H), 5.09-5.13 (m, 1H), 6.36 (s, 1H), 6.58 (d, J=8.88 Hz, 1H), 7.16 (d, J=7.63 Hz, 1H), 7.29 (d, J=7.50 Hz, 1H), 7.41-7.46 (m, 1H), 8.18 (d, J=8.63 Hz, 1H), 8.25 (s, 1H), 8.43 (s, 1H), 10.05 (s, 1H), 10.98 (s, 1H), 11.34 (s, 1H). Mass spec m/z 775.4 [M+H]⁺.

### Example 127 was Synthesised Following Scheme 124

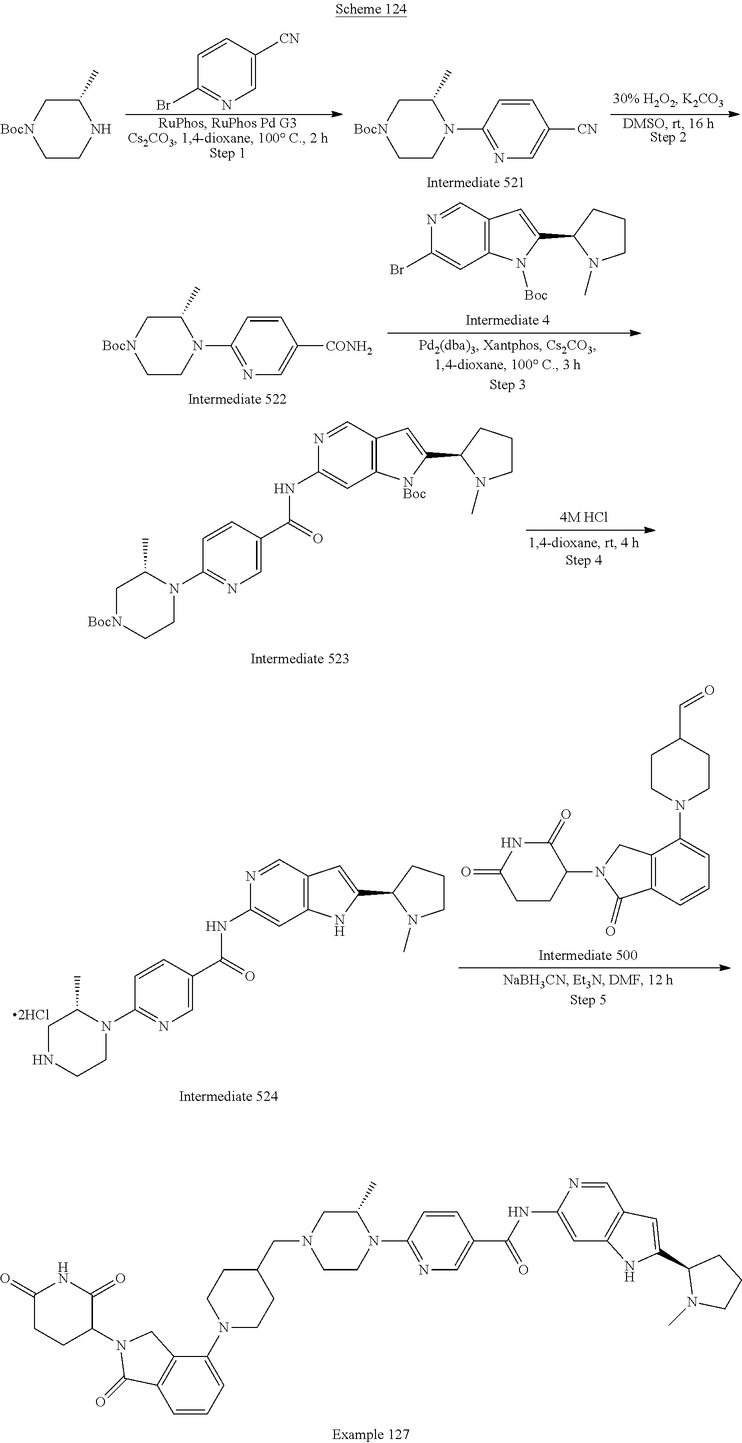

### Step 1

### Intermediate 521: tert-butyl (S)-4-(5-cyanopyridin-2-yl)-3-methylpiperazine-1-carboxylate

To a solution of tert-butyl (S)-3-methylpiperazine-1-carboxylate (CAS No. 147081-29-6, 2.0 g, 9.98 mmol) in 1,4-dioxane (20 mL) was added 6-bromonicotinonitrile (CAS No. 139585-70-9, 1.82 g, 9.98 mmol) followed by cesium carbonate (9.73 g, 29.93 mmol). The reaction mixture was purged with argon for 15 min, then RuPhos (0.95 g, 1.99 mmol) and RuPhos Pd G3 (1.70 g, 1.99 mmol) were added. The reaction mixture was purged with argon for another 10 min and stirred at 100° C. for 2 h. The reaction mixture was concentrated in vacuo and the crude material was purified by combi-flash chromatography, by eluting with 60% ethyl acetate in heptane, to afford tert-butyl (S)-4-(5-cyanopyridin-2-yl)-3-methylpiperazine-1-carboxylate (Intermediate 521, 0.8 g, 26%) as a pale yellow solid. ¹H NMR (400 MHz, DMSO-d₆) δ ppm 0.94-0.94 (m, 3H), 1.42 (s, 9H), 2.94-3.02 (m, 1H), 3.21-3.18 (m, 2H), 3.76-3.81 (m, 1H), 3.90-3.97 (m, 1H), 4.13-4.17 (m, 1H), 4.59-4.62 (m, 1H), 6.88 (d, J=9.2 Hz, 1H), 7.87 (dd, J=9.2, 2.4 Hz, 1H), 5.81 (d, J=2.4 Hz, 1H). Mass spec m/z 303.1 [M+H]⁺.

### Step 2

### Intermediate 522: tert-butyl (S)-4-(5-carbamoylpyridin-2-yl)-3-methylpiperazine-1-carboxylate

To a solution of (S)-4-(5-cyanopyridin-2-yl)-3-methylpiperazine-1-carboxylate (Intermediate 521, 0.8 g, 2.64 mmol) in dimethyl sulfoxide (8.0 mL) was added potassium carbonate (0.73 g, 5.29 mmol) followed by 30% hydrogen peroxide (0.24 mL, 7.93 mmol) and the reaction mixture was stirred at room temperature for 16 h. The reaction mixture was poured into ice cold water (100 mL), the resultant precipitate was collected by filtration, washed with water (50 mL) and dried in vacuo to afford tert-butyl (S)-4-(5-carbamoylpyridin-2-yl)-3-methylpiperazine-1-carboxylate (Intermediate 522, 0.7 g, 83%) as an off-white solid which was used for the next step without further purification. ¹H NMR (400 MHz, DMSO-d₆) δ ppm 0.93-0.98 (m, 3H), 1.42 (s, 9H), 2.95-3.16 (m, 3H), 3.87-3.82 (m, 1H), 3.90-3.99 (m, 1H), 4.09-4.12 (m, 1H), 4.56-4.60 (m, 1H), 6.78 (d, J=9.2 Hz, 1H), 7.14 (s, 1H), 7.76 (s, 1H), 7.97 (dd, J=8.8, 2.4 Hz, 1H), 8.62 (d, J=2.4 Hz, 1H). Mass spec m/z 321.3 [M+H]⁺.

### Step 3

### Intermediate 523: tert-butyl 6-(6-((S)-4-(tert-butoxycarbonyl)-2-methylpiperazin-1-yl)nicotinamido)-2-((R)-1-methylpyrrolidin-2-yl)-1H-pyrrolo[3,2-c]pyridine-1-carboxylate

To a solution of tert-butyl (S)-4-(5-carbamoylpyridin-2-yl)-3-methylpiperazine-1-carboxylate (Intermediate 522, 0.5 g, 1.56 mmol) 1,4-dioxane (8.0 ml) was added tert-butyl (R)-6-bromo-2-(1-methylpyrrolidin-2-yl)-1H-pyrrolo[3,2-c]pyridine-1-carboxylate (Intermediate 4, 0.65 g, 1.87 mmol) followed by cesium carbonate (1.53 g, 4.68 mmol). The reaction mixture was purged with argon for 15 min then Xantphos (0.27 g, 0.36 mmol) and Pd₂(dba)₃ (0.22 g, 0.23 mmol) were added. The reaction mixture was purged with argon for another 10 min and stirred at 100° C. for 3 h. The reaction mixture was concentrated in vacuo and the crude material was purified by combi-flash chromatography, by eluting with 80-100% ethyl acetate in heptane, to afford tert-butyl 6-(6-((S)-4-(tert-butoxycarbonyl)-2-methylpiperazin-1-yl)nicotinamido)-2-((R)-1-methylpyrrolidin-2-yl)-1H-pyrrolo[3,2-c]pyridine-1-carboxylate (Intermediate 523, 0.6 g, 62%) as a yellow solid. ¹H NMR (400 MHz, DMSO-d₆) δ ppm 1.07-1.09 (m, 3H), 1.43 (s, 9H), 1.59-1.79 (m, 13H), 2.33-2.36 (m, 5H), 3.09-3.18 (m, 3H), 3.79-3.84 (m, 1H), 3.89-3.96 (m, 2H), 4.15-4.19 (m, 1H), 4.61-4.64 (m, 1H), 6.74 (s, 1H), 6.80-6.84 (m, 1H), 8.15-8.19 (s, 1H), 8.57 (s, 1H), 8.82 (s, 1H), 8.90 (s, 1H), 10.52 (s, 1H). Mass spec m/z 620.3 [M+H]⁺.

### Step 4

### Intermediate 524: 6-((S)-2-methylpiperazin-1-yl)-N-(2-((R)-1-methylpyrrolidin-2-yl)-1H-pyrrolo[3,2-c]pyridin-6-yl)nicotinamide dihydrochloride

To a stirred solution of tert-butyl 6-(6-((S)-4-(tert-butoxycarbonyl)-2-methylpiperazin-1-yl)nicotinamido)-2-((R)-1-methylpyrrolidin-2-yl)-1H-pyrrolo[3,2-c]pyridine-1-carboxylate (Intermediate 523, 0.5 g, 0.81 mmol) in 1,4-dioxane (3 mL) was added 4M hydrochloric acid in 1,4-dioxane (1 mL, 4.03 mmol) and the reaction mixture was stirred at room temperature for 4 h. The reaction mixture was then concentrated in vacuo to afford 6-((S)-2-methylpiperazin-1-yl)-N-(2-((R)-1-methylpyrrolidin-2-yl)-1H-pyrrolo[3,2-c]pyridin-6-yl)nicotinamide dihydrochloride (Intermediate 524, 0.35 g) as an off-white solid which was used for the next step without further purification. Mass spec m/z 418.1 [M−H]⁻.

### Step 5

### Example 127: 6-((2S)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)nicotinamide

To a solution of 6-((S)-2-methylpiperazin-1-yl)-N-(2-((R)-1-methylpyrrolidin-2-yl)-1H-pyrrolo[3,2-c]pyridin-6-yl)nicotinamide dihydrochloride (Intermediate 524, 0.34 g, 0.76 mmol) in dimethylformamide (3.0 mL) was added a solution of 1-(2-(2,6-dioxopiperidin-3-yl)-1-oxoisoindolin-4-yl)piperidine-4-carbaldehyde (Intermediate 500, 0.30 g, 0.63 mmol) in dimethylformamide (2 mL), followed by triethylamine (0.26 mL, 1.91 mmol). The reaction mixture was heated at 70° C. for 2 h. Sodium cyanoborohydride (0.12 g, 1.91 mmol) was then added and the reaction mixture was stirred at 70° C. for 12 h. The reaction mixture was poured into water (50 mL), the resultant precipitate was collected by filtration and dried in vacuo. The crude material was purified by preparative HPLC (Method A) to afford 6-((2S)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)nicotinamide (Example 127, 0.025 g, 5%) as an off-white solid. ¹H NMR (400 MHz, DMSO-d₆) δ ppm 1.19-1.32 (m, 5H), 1.69-2.06 (s, 9H), 2.13-2.29 (m, 9H), 2.57-2.61 (m, 1H), 2.71-2.85 (m, 3H), 2.77-2.98 (m, 2H), 3.07-3.18 (m, 2H), 3.37-3.45 (m, 2H), 4.17-4.22 (m, 1H), 4.27-4.32 (m, 1H), 4.42-4.47 (m, 1H), 4.57-4.63 (m, 1H), 5.09-5.15 (m, 1H), 6.38 (s, 1H), 6.81 (d, J=9.2 Hz, 1H), 7.16 (d, J=7.6 Hz, 1H), 7.29 (d, J=7.2 Hz, 1H), 7.43 (t, J=7.6 Hz, 1H), 8.15-8.18 (m, 2H), 8.48 (s, 1H), 8.81 (s, 1H), 10.26 (s, 1H), 10.98 (s, 1H), 11.33 (s, 1H). Mass spec m/z 759.3 [M+H]⁺.

### Example 128 was Synthesised Following Scheme 125

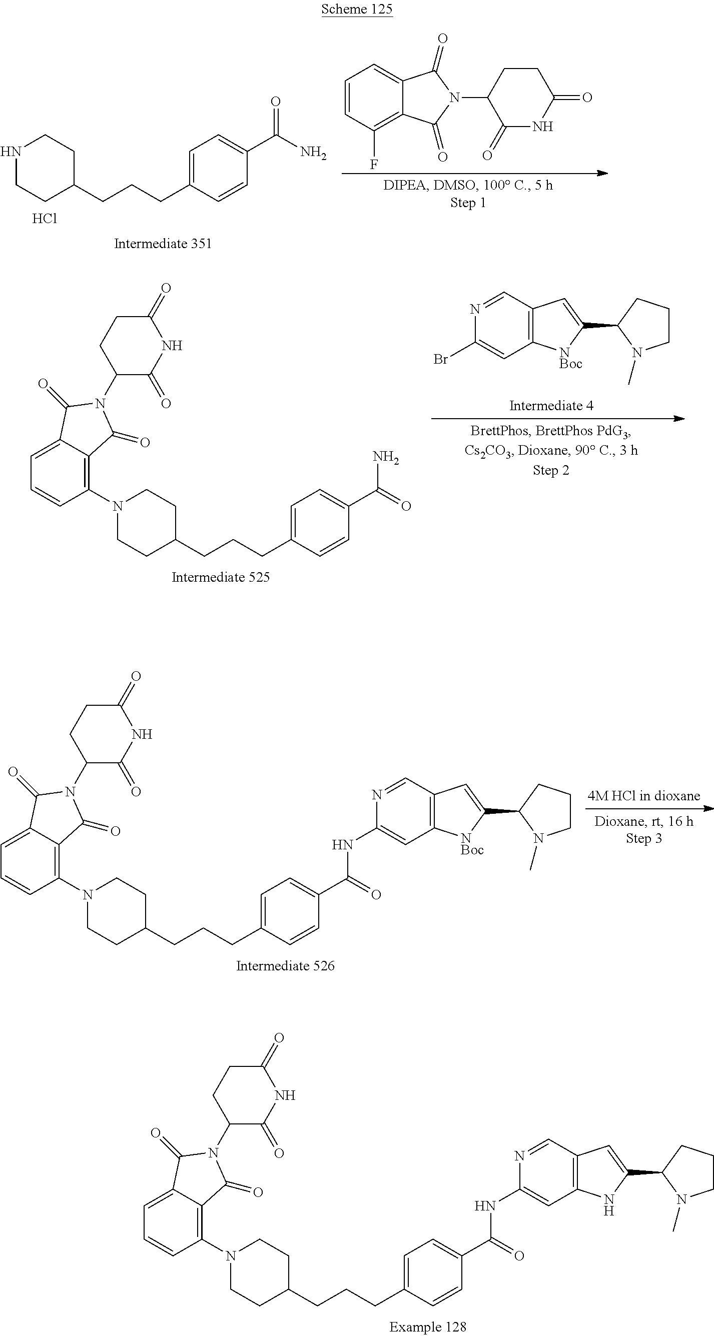

### Step 1

### Intermediate 525: 4-(3-(1-(2-(2,6-dioxopiperidin-3-yl)-1,3-dioxoisoindolin-4-yl)piperidin-4-yl)propyl)benzamide

To a solution of 4-(3-(piperidin-4-yl)propyl)benzamide hydrochloride (Intermediate 351, 1.0 g, 3.50 mmol) in dimethyl sulfoxide (10 mL) was added 2-(2,6-dioxopiperidin-3-yl)-4-fluoroisoindoline-1,3-dione (CAS No: 835616-60-9, 0.98 g, 3.50 mmol) followed by N,N-diisopropylethylamine (2.6 mL, 14.0 mmol) and the reaction mixture was heated at 100° C. for 5 h. After completion, the reaction mixture was diluted with water (300 mL) and extracted with ethyl acetate (3×200 mL). The combined organic layers were separated, dried over anhydrous Na₂SO₄ and concentrated in vacuo to afford 4-(3-(1-(2-(2,6-dioxopiperidin-3-yl)-1,3-dioxoisoindolin-4-yl)piperidin-4-yl)propyl)benzamide (Intermediate 525, 0.70 g, 39%) as brown solid, which was used for the next step without further purification. ¹H NMR (400 MHz, DMSO-d₆) δ ppm 1.23-1.34 (m, 4H), 1.63-1.75 (m, 4H), 2.27-2.32 (m, 2H), 2.54-2.60 (m, 6H), 2.61-2.65 (m, 1H), 3.05-3.08 (m, 1H), 3.21-3.26 (m, 1H), 3.81-3.95 (m, 1H), 7.13-7.32 (m, 4H), 7.77-7.79 (m, 1H), 7.81-7.87 (m, 2H), 10.77 (br s, 1H).

### Step 2

### Intermediate 526: tert-butyl 6-(4-(3-(1-(2-(2,6-dioxopiperidin-3-yl)-1,3-dioxoisoindolin-4-yl)piperidin-4-yl)propyl)benzamido)-2-((R)-1-methylpyrrolidin-2-yl)-1H-pyrrolo[3,2-c]pyridine-1-carboxylate

To a solution of 4-(3-(1-(2-(2,6-dioxopiperidin-3-yl)-1,3-dioxoisoindolin-4-yl)piperidin-4-yl)propyl)benzamide (Intermediate 525, 0.30 g, 0.59 mmol) in 1,4-dioxane (6 mL) were added tert-butyl (R)-6-bromo-2-(1-methylpyrrolidin-2-yl)-1H-pyrrolo[3,2-c]pyridine-1-carboxylate (Intermediate 4, 0.27 g, 0.72 mmol) followed by cesium carbonate (0.58 g, 1.79 mmol). The reaction mixture was purged with argon for 15 min then Pd₂(dba)₃ (0.08 g, 0.09 mmol) and Xantphos (0.10 g, 0.18 mmol) were added. The reaction mixture was further purged with argon for 10 min and stirred at 100° C. for 2 h. After completion, the reaction mixture was filtered through a celite bed, the filter pad washed with ethyl acetate (200 mL) and the filtrate concentrated in vacuo. The crude material was purified by combi-flash chromatography, by eluting with 90% ethyl acetate in heptane, to afford tert-butyl 6-(4-(3-(1-(2-(2,6-dioxopiperidin-3-yl)-1,3-dioxoisoindolin-4-yl)piperidin-4-yl)propyl)benzamido)-2-((R)-1-methylpyrrolidin-2-yl)-1H-pyrrolo[3,2-c]pyridine-1-carboxylate (Intermediate 526, 0.3 g, 63%) as a yellow solid. ¹H NMR (400 MHz, DMSO-d₆) δ ppm 1.24-1.35 (m, 3H), 1.53-1.83 (m, 14H), 1.84-1.88 (m, 2H), 1.97-1.95 (m, 2H), 2.28-2.41 (m, 5H), 2.60-2.71 (m, 4H), 2.80-2.89 (m, 3H), 3.10-3.16 (m, 2H), 3.64-3.72 (m, 2H), 3.91-3.96 (m, 1H), 5.06-5.11 (m, 1H), 6.37 (s, 1H), 7.05-7.35 (m, 4H), 7.65-7.70 (m, 1H), 7.98-8.01 (m, 2H), 8.59 (s, 1H), 8.92 (s, 1H), 10.59 (s, 1H), 11.07 (s, 1H).

### Step 3

### Example 128: 4-(3-(1-(2-(2,6-dioxopiperidin-3-yl)-1,3-dioxoisoindolin-4-yl)piperidin-4-yl)propyl)-N-(2-((R)-1-methylpyrrolidin-2-yl)-1H-pyrrolo[3,2-c]pyridin-6-yl)benzamide

To a cooled (0° C.) solution of tert-butyl 6-(4-(3-(1-(2-(2,6-dioxopiperidin-3-yl)-1,3-dioxoisoindolin-4-yl)piperidin-4-yl)propyl)benzamido)-2-((R)-1-methylpyrrolidin-2-yl)-1H-pyrrolo[3,2-c]pyridine-1-carboxylate (Intermediate 526, 0.3 g, 0.37 mmol) in 1,4-dioxane (6 mL) was added 4M hydrochloric acid in 1,4-dioxane (3 mL) and the reaction mixture was stirred at room temperature for 16 h. The reaction mixture was then concentrated in vacuo and the crude material purified by preparative HPLC (Method A) to afford 4-(3-(1-(2-(2,6-dioxopiperidin-3-yl)-1,3-dioxoisoindolin-4-yl)piperidin-4-yl)propyl)-N-(2-((R)-1-methylpyrrolidin-2-yl)-1H-pyrrolo[3,2-c]pyridin-6-yl)benzamide (Example 128, 0.03 g, 12%) as an off-white solid. ¹H NMR (400 MHz, DMSO-d₆) δ ppm 1.26-1.38 (m, 4H), 1.41-1.50 (m, 1H), 1.63-1.71 (m, 2H), 1.74-1.81 (m, 2H), 1.98-2.06 (m, 1H), 2.11-2.26 (m, 2H), 2.46-2.48 (m, 1H), 2.52-2.61 (m, 2H), 2.68-2.71 (m, 2H), 2.82-2.89 (m, 6H), 3.24-3.31 (m, 1H), 3.66-3.71 (m, 4H), 4.62-4.67 (m, 1H), 5.04-5.12 (m, 1H), 6.96 (s, 1H), 7.31-7.34 (m, 2H), 7.39 (d, J=8.40 Hz, 2H), 7.67 (d, J=8.40 Hz, 1H), 7.99 (d, J=8.40 Hz, 2H), 8.82 (br s, 1H), 10.03 (s, 1H), 10.87 (br s, 1H), 11.09 (s, 1H), 12.13 (s, 1H). Mass spec m/z 702.0 [M+H]⁺.

### Example 129 was Synthesised Following Scheme 126

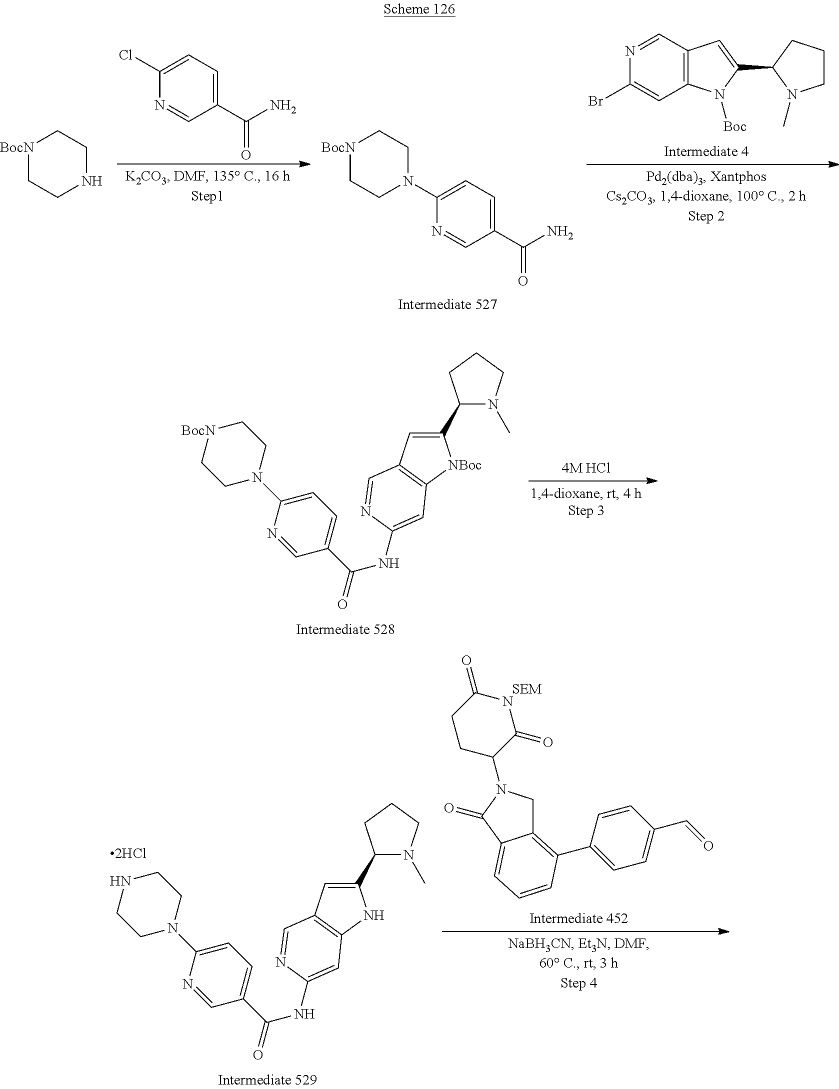

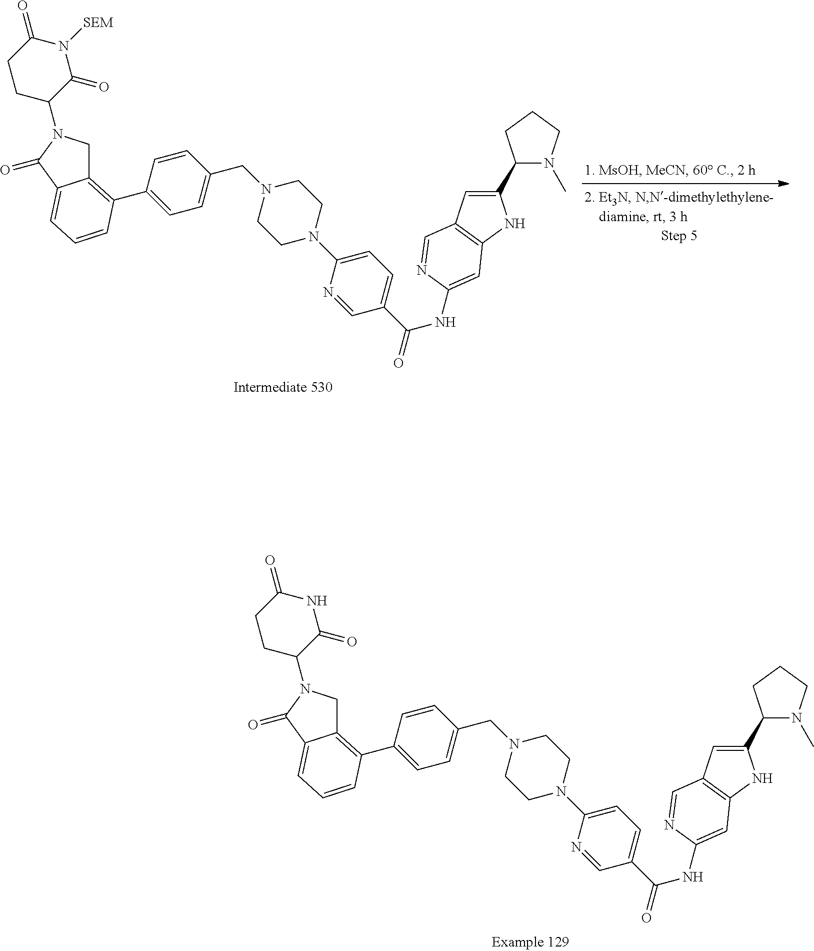 [SMILES: "SEM" ; CN1CCC[C@@H]1c1cc2cnc(NC(=O)c3ccc(N4CCN(Cc5ccc(-c6cccc7c6CN(C6CCC(=O)NC6=O)C7=O)cc5)CC4)nc3)cc2[nH]1 ; CN1CCC[C@@H]1c1cc2cnc(NC(=O)c3ccc(N4CCN(Cc5ccc(-c6cccc7c6CN(C6CCC(=O)NC6=O)C7=O)cc5)CC4)nc3)cc2[nH]1]

### Step 1

### Intermediate 527: tert-butyl 4-(5-carbamoylpyridin-2-yl)piperazine-1-carboxylate

To a solution of tert-butyl piperazine-1-carboxylate (CAS No: 57260-71-6, 6.0 g, 32.22 mmol) in dimethylformamide (60 mL) was added 6-chloronicotinamide (CAS No: 6271-78-9, 4.54 g, 28.99 mmol) followed by potassium carbonate (17.83 g, 128.86 mmol) and the resulting reaction mixture was heated at 135° C. for 16 h. After completion, the reaction mixture was quenched with ice cold water (600 mL), the resultant precipitate was collected by filtration and dried in vacuo to afford tert-butyl 4-(5-carbamoylpyridin-2-yl)piperazine-1-carboxylate (Intermediate 527, 5.50 g) as an off-white solid, which was used in the next step without further purification. ¹H NMR (400 MHz, DMSO-d₆) δ ppm 1.42 (s, 9H), 3.40-3.43 (m, 4H), 3.59-3.61 (m, 4H), 6.85 (d, J=8.80 Hz, 1H), 7.17 (br s, 1H), 7.80 (br s, 1H), 7.96 (dd, J=2.40, 8.80 Hz, 1H), 8.62 (d, J=2.40 Hz, 1H). Mass spec m/z 307.1 [M+H]⁺.

### Step 2

### Intermediate 528: tert-butyl (R)-6-(6-(4-(tert-butoxycarbonyl)piperazin-1-yl)nicotinamido)-2-(1-methylpyrrolidin-2-yl)-1H-pyrrolo[3,2-c]pyridine-1-carboxylate

To a stirred suspension of tert-butyl 4-(5-carbamoylpyridin-2-yl)piperazine-1-carboxylate (Intermediate 527, 1.0 g, 3.26 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 1.37 g, 3.60 mmol) followed by cesium carbonate (3.19 g, 9.80 mmol). The reaction mixture was purged with argon for 15 min then Pd₂(dba)₃ (0.45 g, 0.49 mmol) and Xantphos (0.57 g, 0.98 mmol) were added. The reaction mixture was purged with argon for another 10 min and then stirred at 100° C. for 2 h. After completion, the reaction mixture was concentrated in vacuo to obtain the crude compound. The crude material was purified by combi-flash chromatography, by eluting with 90% ethyl acetate in heptane, to afford tert-butyl (R)-6-(6-(4-(tert-butoxycarbonyl)piperazin-1-yl)nicotinamido)-2-(1-methylpyrrolidin-2-yl)-1H-pyrrolo[3,2-c]pyridine-1-carboxylate (Intermediate 528, 1.40 g, 43%) as a yellow solid. ¹H NMR (400 MHz, DMSO-d₆) δ ppm 1.43 (s, 9H), 1.58-1.66 (m, 2H), 1.69 (s, 9H), 1.72-1.76 (m, 2H), 2.30-2.33 (m, 2H), 2.35 (s, 3H), 3.11-3.14 (m, 1H), 3.42-3.45 (m, 4H), 3.62-3.64 (m, 3H), 3.88-3.94 (m, 1H), 6.74 (s, 1H), 6.88 (d, J=9.2 Hz, 1H), 8.18 (d, J=8.0 Hz, 1H), 8.57 (s, 1H), 8.81 (s, 1H), 8.91 (s, 1H), 10.53 (br s, 1H). Mass spec m/z 606.4 [M+H]⁺.

### Step 3

### Intermediate 529: (R)—N-(2-(1-methylpyrrolidin-2-yl)-1H-pyrrolo[3,2-c]pyridin-6-yl)-6-(piperazin-1-yl)nicotinamide dihydrochloride

To a cooled (0° C.) solution of tert-butyl (R)-6-(6-(4-(tert-butoxycarbonyl)piperazin-1-yl)nicotinamido)-2-(1-methylpyrrolidin-2-yl)-1H-pyrrolo[3,2-c]pyridine-1-carboxylate (Intermediate 528, 1.30 g, 2.10 mmol) in 1,4-dioxane (13 mL) was added 4M hydrochloric acid (13 mL) and the reaction mixture was stirred at room temperature for 4 h. After completion, the reaction mixture was concentrated in vacuo. The resultant residue was triturated with ether (20 mL) and dried in vacuo to afford (R)—N-(2-(1-methylpyrrolidin-2-yl)-1H-pyrrolo[3,2-c]pyridin-6-yl)-6-(piperazin-1-yl)nicotinamide dihydrochloride (Intermediate 529, 1.6 g) as an off-white solid, which was used into next step without purification. Mass spec m/z 406.1 [M+H]⁺.

### Step 4

### Intermediate 530: 6-(4-(4-(2-(2,6-dioxo-1-((2-(trimethylsilyl)ethoxy)methyl)piperidin-3-yl)-1-oxoisoindolin-4-yl)benzyl)piperazin-1-yl)-N-(2-((R)-1-methylpyrrolidin-2-yl)-1H-pyrrolo[3,2-c]pyridin-6-yl)nicotinamide

To a solution of 4-(2-(2,6-dioxo-1-((2-(trimethylsilyl)ethoxy)methyl)piperidin-3-yl)-1-oxoisoindolin-4-yl)benzaldehyde (Intermediate 452, 0.5 g, 1.05 mmol) in dimethylformamide (10 mL) was added triethylamine (0.81 mL, 4.2 mmol) followed by (R)—N-(2-(1-methylpyrrolidin-2-yl)-1H-pyrrolo[3,2-c]pyridin-6-yl)-6-(piperazin-1-yl)nicotinamide dihydrochloride (Intermediate 529, 0.46 g, 1.05 mmol) and the reaction mixture was stirred at 60° C. for 2 h. Sodium cyanoborohydride (0.2 g, 3.13 mmol) was then added and resulting reaction mixture was stirred at room temperature for 3 h. After completion, the reaction mixture was quenched with ice cold water (100 mL), the resultant precipitate was collected by filtration and dried in vacuo to afford to afford 6-(4-(4-(2-(2,6-dioxo-1-((2-(trimethylsilyl)ethoxy)methyl)piperidin-3-yl)-1-oxoisoindolin-4-yl)benzyl)piperazin-1-yl)-N-(2-((R)-1-methylpyrrolidin-2-yl)-1H-pyrrolo[3,2-c]pyridin-6-yl)nicotinamide (Intermediate 530, 0.6 g) as an off-white solid, which was used into next step without purification. Mass spec m/z 868.4 [M+H+2]⁺.

### Step 5

### Example 129: 6-(4-(4-(2-(2,6-dioxopiperidin-3-yl)-1-oxoisoindolin-4-yl)benzyl)piperazin-1-yl)-N-(2-((R)-1-methylpyrrolidin-2-yl)-1H-pyrrolo[3,2-c]pyridin-6-yl)nicotinamide

To a solution of 6-(4-(4-(2-(2,6-dioxo-1-((2-(trimethylsilyl)ethoxy)methyl)piperidin-3-yl)-1-oxoisoindolin-4-yl)benzyl)piperazin-1-yl)-N-(2-((R)-1-methylpyrrolidin-2-yl)-1H-pyrrolo[3,2-c]pyridin-6-yl)nicotinamide (Intermediate 530, 0.550 g, 0.63 mmol) in acetonitrile (7.0 mL) was added methanesulfonic acid (0.61 g, 6.30 mmol) at room temperature and the reaction mixture was stirred at 60° C. for 2 h. After cooling to room temperature, N,N′-dimethylethylenediamine (0.28 g, 3.17 mmol) followed by triethylamine (1.28 g, 12.67 mmol) were added and the reaction stirred at room temperature for 3 h. After completion, the reaction mixture was concentrated in vacuo, diluted with water (50 mL) and the resultant precipitate was collected by filtration and dried in vacuo. The crude material was purified by preparative HPLC (Method A) to afford 6-(4-(4-(2-(2,6-dioxopiperidin-3-yl)-1-oxoisoindolin-4-yl)benzyl)piperazin-1-yl)-N-(2-((R)-1-methylpyrrolidin-2-yl)-1H-pyrrolo[3,2-c]pyridin-6-yl)nicotinamide (Example 129, 0.108 g, 23%) as an off-white solid. ¹H NMR (400 MHz, DMSO-d₆) δ ppm 1.78-1.93 (m, 4H), 1.97-2.02 (m, 1H), 2.12-2.14 (m, 1H), 2.16 (s, 3H), 2.21-2.8 (m, 1H), 2.48-2.52 (m, 1H), 2.52-2.56 (m, 2H), 2.58-2.61 (m, 1H), 2.88-2.94 (m, 1H), 3.12-3.16 (m, 1H), 3.28-3.32 (m, 2H), 3.60 (s, 2H), 3.65-3.68 (m, 4H), 4.41-4.47 (m, 1H), 4.63-4.67 (m, 1H), 5.13-5.17 (m, 1H), 6.38 (s, 1H), 6.88 (d, J=1.60 Hz, 1H), 7.48 (d, J=8.0 Hz, 2H), 7.59 (d, J=8.0 Hz, 2H), 7.65 (d, J=7.60 Hz, 1H), 7.73-7.78 (m, 2H), 8.16-8.19 (m, 2H), 8.48 (s, 1H), 8.80 (s, 1H), 10.30 (s, 1H), 10.98 (s, 1H), 11.36 (s, 1H). Mass spec m/z 738.0 [M+H]⁺.

### Example 130 was Synthesised Following Scheme 127

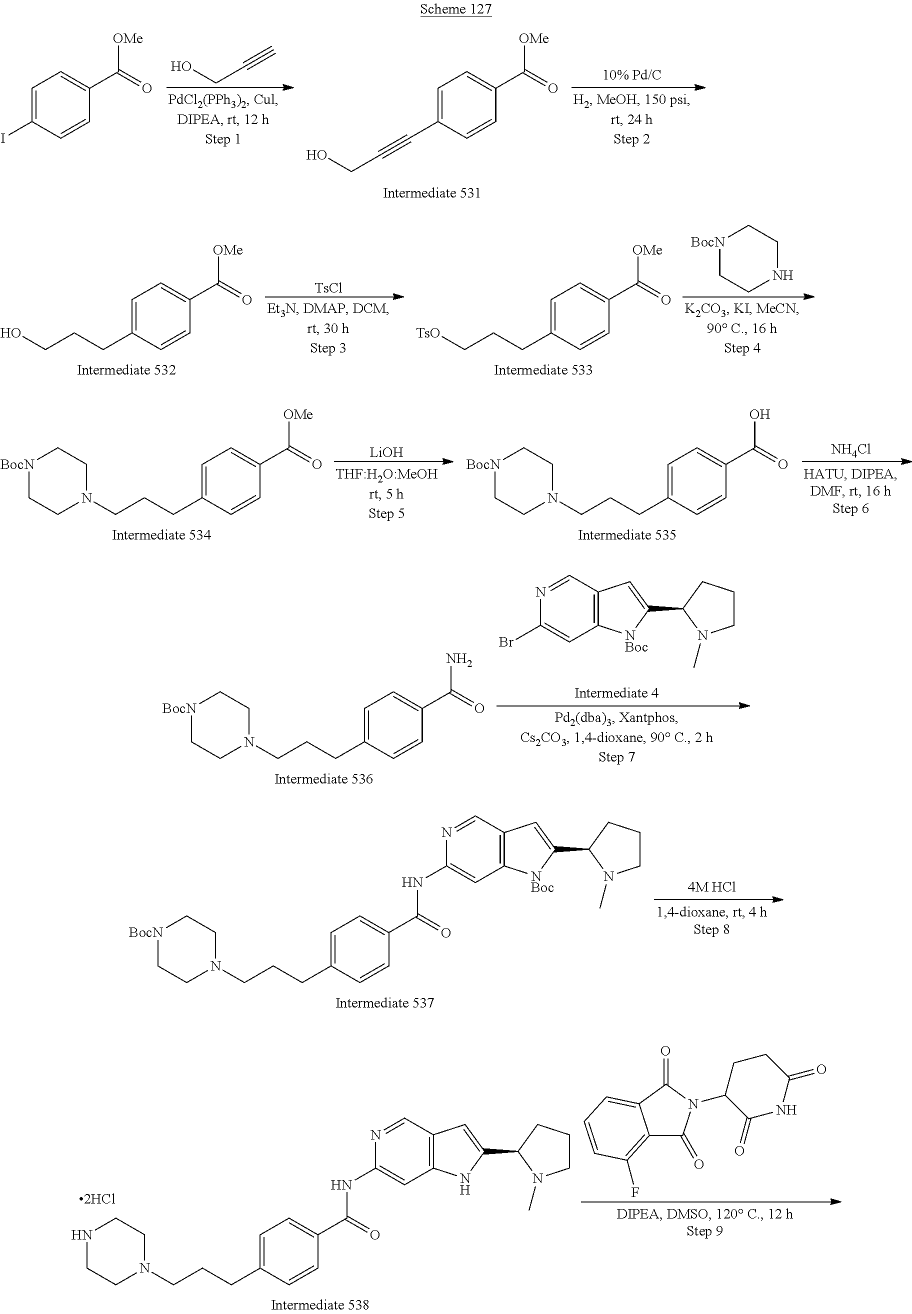

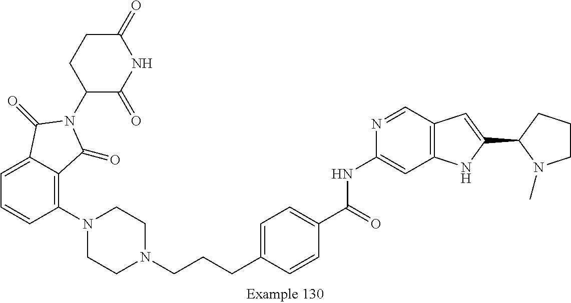 [SMILES: CN1CCC[C@@H]1c1cc2cnc(NC(=O)c3ccc(CCCN4CCN(c5cccc6c5C(=O)N(C5CCC(=O)NC5=O)C6=O)CC4)cc3)cc2[nH]1]

### Step 1

### Intermediate 531: methyl 4-(3-hydroxyprop-1-yn-1-yl)benzoate

To a suspension of methyl 4-iodobenzoate (CAS No. 619-44-3, 6.0 g, 22.89 mmol) in triethylamine (114.0 mL, 814.0 mmol) was added prop-2-yn-1-ol (CAS No. 107-19-7, 1.54 g, 27.47 mmol). The reaction mixture was purged with argon gas for 15 min then PdCl₂(PPh₃)₂ (0.32 g, 0.45 mmol) and copper(I) iodide (0.17 g, 0.91 mmol) were added. The reaction mixture was purged with argon for another 10 min and stirred at room temperature for 12 h. After completion, the reaction mixture was concentrated in vacuo, diluted with ethyl acetate (100 mL) and washed with saturated ammonium chloride (50 mL). The organic layer was separated, dried over anhydrous Na₂SO₄, filtered, and concentrated in vacuo. The crude material was purified by combi-flash chromatography, by eluting with 50% ethyl acetate in heptane, to afford methyl 4-(3-hydroxyprop-1-yn-1-yl)benzoate (Intermediate 531, 4.0 g, 92%) as a brown solid. ¹H NMR (400 MHz, CDCl₃) δ ppm 1.71-1.74 (m 1H), 3.91 (s, 3H), 4.49-4.54 (m, 2H), 7.49 (d, J=7.93 Hz, 2H), 7.98 (d, J=8.31 Hz, 2H). Mass spec m/z 191.1 [M+H]⁺.

### Step 2

### Intermediate 532: methyl 4-(3-hydroxypropyl)benzoate

To a solution of methyl 4-(3-hydroxyprop-1-yn-1-yl)benzoate (Intermediate 531, 8.00 g, 42.06 mmol) in methanol (200 mL) was added 10% Pd/C (2.0 g, 18.79 mmol). The mixture was stirred at room temperature for 24 h under hydrogen atmosphere at 150 psi. After completion, the reaction mixture was concentrated in vacuo to afford methyl 4-(3-hydroxypropyl)benzoate (Intermediate 532, 7.50 g, 91.8%) as an orange liquid, which was used for the next step without further purification. ¹H NMR (400 MHz, DMSO-d₆) δ ppm 1.70-1.74 (m, 2H), 2.66-2.70 (m, 2H), 3.38-3.42 (m, 2H), 3.83 (s, 3H), 4.47-4.50 (m, 1H), 7.35 (d, J=8.22 Hz, 2H), 7.87 (d, J=7.83 Hz, 2H). Mass spec m/z 195.2 [M+H]⁺.

### Step 3

### Intermediate 533: methyl 4-(3-(tosyloxy)propyl)benzoate

To a cooled (0° C.) solution of methyl 4-(3-hydroxypropyl)benzoate (Intermediate 532, 5.00 g, 25.74 mmol) in dichloromethane (50 mL) was added triethylamine (19.0 mL, 128.71 mmol) followed by tosyl chloride (7.74 g, 38.61 mmol) and 4-dimethylaminopyridine (0.33 g, 2.57 mmol). The reaction mixture was stirred at room temperature for 3 h. After completion, the reaction mixture was diluted with ice cold water (50 mL) and extracted with ethyl acetate (2×100 mL). The combined organic layers were dried over anhydrous Na₂SO₄, filtered, and concentrated in vacuo. The crude material was purified by combi-flash chromatography, by eluting with 50% ethyl acetate in heptane, to afford methyl 4-(3-(tosyloxy)propyl)benzoate (Intermediate 533, 7.5 g, 84%) as a white solid. ¹H NMR (400 MHz, DMSO-d₆) δ ppm 1.87-1.91 (m, 2H), 2.42 (s, 3H), 2.61-2.65 (m, 2H), 3.83 (s, 3H), 3.97-4.00 (m, 2H), 7.21-7.25 (m, 2H), 7.47 (d, J=7.83 Hz, 2H), 7.76-7.82 (m, 4H). Mass spec m/z 349.0 [M+H]⁺.

### Step 4

### Intermediate 534: tert-butyl 4-(3-(4-(methoxycarbonyl)phenyl)propyl)piperazine-1-carboxylate

To a solution of methyl 4-(3-(tosyloxy)propyl)benzoate (Intermediate 533, 8.50 g, 24.0 mmol) in acetonitrile (20 mL) was added potassium carbonate (10.0 g, 73.0 mmol) and potassium iodide (0.81 g, 4.9 mmol) and the reaction mixture was stirred at room temperature for 15 min. tert-Butyl piperazine-1-carboxylate (CAS No. 57260-71-6, 5.5 g, 29.0 mmol) was then added and the reaction mixture heated at 90° C. for 16 h. After completion, the reaction mixture was diluted with water (50 mL) and extracted with ethyl acetate (2×70 mL). The combined organic layers were dried over anhydrous Na₂SO₄, filtered, and concentrated in vacuo. The crude material was purified by combi-flash chromatography, by eluting with 50% ethyl acetate in heptane, to afford tert-butyl 4-(3-(4-(methoxycarbonyl)phenyl)propyl)piperazine-1-carboxylate (Intermediate 534, 7.5 g, 85%) as a colourless liquid. ¹H NMR (400 MHz, DMSO-d₆) δ ppm 1.38 (s, 9H), 1.72-1.77 (m, 2H), 2.21-2.31 (m, 6H), 2.64-2.68 (m, 2H), 3.26-3.30 (m, 4H), 3.83 (s, 3H), 7.36 (d, J=8.31 Hz, 2H), 7.87 (d, J=7.93 Hz, 2H). Mass spec m/z 363.1 [M+H]⁺.

### Step 5

### Intermediate 535: 4-(3-(4-(tert-butoxycarbonyl)piperazin-1-yl)propyl)benzoic acid

To a solution of tert-butyl 4-(3-(4-(methoxycarbonyl)phenyl)propyl)piperazine-1-carboxylate (Intermediate 534, 7.0 g, 19.0 mmol) in methanol: tetrahydrofuran: water (1:1:1, 105 mL) was added lithium hydroxide (2.4 g, 97.0 mmol) and the reaction mixture was stirred at room temperature for 5 h. After completion, the reaction mixture was concentrated in vacuo, acidified to pH~4 by dropwise addition of saturated solution of citric acid (50 mL) and extracted with ethyl acetate (2×100 mL). The combined organic layers were dried over anhydrous Na₂SO₄, filtered, and concentrated in vacuo to afford 4-(3-(4-(tert-butoxy carbonyl)piperazin-1-yl)propyl)benzoic acid (Intermediate 535, 5.50 g, 82%) as an off-yellow solid, which was used for the next step without further purification. ¹H NMR (400 MHz, DMSO-d₆) δ ppm 1.15-1.18 (m, 1H), 1.38 (s, 9H), 1.73-1.76 (m, 2H), 2.25-2.36 (m, 6H), 2.63-2.67 (m, 2H), 3.27-3.34 (m, 4H), 7.32 (d, J=8.25 Hz, 2H), 7.85 (d, J=8.25 Hz, 2H), 12.59 (br s, 1H). Mass spec m/z 349.3 [M+H]⁺.

### Step 6

### Intermediate 536: tert-butyl 4-(3-(4-carbamoylphenyl)propyl)piperazine-1-carboxylate

To a solution of 4-(3-(4-(tert-butoxy carbonyl)piperazin-1-yl)propyl)benzoic acid (Intermediate 535, 5.50 g, 16.0 mmol) in dimethylformamide (10 mL) was added HATU (12.0 g, 32.0 mmol) and the reaction mixture was stirred at room temperature for 20 min. Ammonium chloride (8.4 g, 160.0 mmol) and N,N-diisopropylethylamine (22.0 mL, 130 mmol) were then added and the reaction mixture was stirred at room temperature for 16 h. After completion, saturated aqueous citric acid solution was added to adjust the pH to ~3, then the mixture was extracted with ethyl acetate (2×100 mL). The combined organic layers were dried over anhydrous Na₂SO₄, filtered, and concentrated in vacuo to afford tert-butyl 4-(3-(4-carbamoylphenyl)propyl)piperazine-1-carboxylate (Intermediate 536, 3.4 g, 62%) as an off-white solid, which was used for the next step without further purification. ¹H NMR (400 MHz, DMSO-d₆) δ ppm 1.16-1.27 (m, 2H), 1.39 (s, 9H), 1.70-1.80 (m, 2H), 2.23-2.37 (m, 6H), 2.61-2.65 (m, 2H), 3.08-3.25 (m, 4H), 7.24 (br s, 1H), 7.27 (d, J=7.93 Hz, 2H), 7.78 (d, J=7.55 Hz, 2H), 7.87 (br s, 1H). Mass spec m/z 348.1 [M+H]⁺.

### Step 7

### Intermediate 537: tert-butyl (R)-6-(4-(3-(4-(tert-butoxycarbonyl)piperazin-1-yl)propyl)benzamido)-2-(1-methylpyrrolidin-2-yl)-1H-pyrrolo[3,2-c]pyridine-1-carboxylate

To a solution of tert-butyl 4-(3-(4-carbamoylphenyl)propyl)piperazine-1-carboxylate (Intermediate 536, 0.55 g, 1.58 mmol) in 1,4-dioxane (5 mL) was added tert-butyl 6-bromo-2-(1-methylpyrrolidin-2-yl)pyrrolo[3,2-c]pyridine-1-carboxylate (Intermediate 4, 0.72 g, 1.90 mmol) and cesium carbonate (1.55 g, 4.75 mmol). The reaction mixture was purged with argon for 15 min then Pd₂(dba)₃ (0.22 g, 0.23 mmol) and Xantphos (0.28 g, 0.47 mmol) were added. The reaction mixture was further purged with argon for 10 min and heated at 90° C. for 2 h. After completion, reaction mixture was filtered and concentrated in vacuo. The crude material was purified by combi-flash chromatography, by eluting with 15% MeOH in DCM, to afford tert-butyl (R)-6-(4-(3-(4-(tert-butoxycarbonyl)piperazin-1-yl)propyl)benzamido)-2-(1-methylpyrrolidin-2-yl)-1H-pyrrolo[3,2-c]pyridine-1-carboxylate (Intermediate 537, 0.50 g, 50%) as a brown solid. ¹H NMR (401 MHz, DMSO-d₆) δ ppm 1.39 (s, 9H), 1.70 (s, 9H), 1.72-1.80 (m, 4H), 2.25-2.34 (m, 7H), 2.36 (s, 3H), 2.37-2.41 (m, 1H), 2.65-2.69 (m, 2H), 3.10-3.17 (m, 1H), 3.26-3.33 (m, 4H), 3.88-3.94 (m, 1H), 5.70-5.73 (m, 1H), 6.75 (s, 1H), 7.34 (d, J=8.31 Hz, 2H), 7.98 (d, J=8.31 Hz, 2H), 8.59 (s, 1H), 8.93 (s, 1H), 10.63 (br s, 1H). Mass spec m/z 647.4 [M+H]⁺.

### Step 8

### Intermediate 538: (R)—N-(2-(1-methylpyrrolidin-2-yl)-1H-pyrrolo[3,2-c]pyridin-6-yl)-4-(3-(piperazin-1-yl)propyl)benzamide dihydrochloride

To a cooled (0° C.) solution of tert-butyl (R)-6-(4-(3-(4-(tert-butoxycarbonyl)piperazin-1-yl)propyl)benzamido)-2-(1-methylpyrrolidin-2-yl)-1H-pyrrolo[3,2-c]pyridine-1-carboxylate (Intermediate 537, 0.50 g, 0.77 mmol) in 1,4-dioxane (2 mL) was added 4 M hydrochloric acid in 1,4-dioxane (5 mL) and the reaction mixture was stirred at room temperature for 4 h. The reaction mixture was concentrated in vacuo to afford (R)—N-(2-(1-methylpyrrolidin-2-yl)-1H-pyrrolo[3,2-c]pyridin-6-yl)-4-(3-(piperazin-1-yl)propyl)benzamide dihydrochloride (Intermediate 538, 0.7 g) as an off-yellow solid, which was used for the next step without further purification. Mass spec m/z 447.0 [M+H]⁺.

### Step 9

### Example 130: 4-(3-(4-(2-(2,6-dioxopiperidin-3-yl)-1,3-dioxoisoindolin-4-yl)piperazin-1-yl)propyl)-N-(2-((R)-1-methylpyrrolidin-2-yl)-1H-pyrrolo[3,2-c]pyridin-6-yl)benzamide

To a solution of (R)—N-(2-(1-methylpyrrolidin-2-yl)-1H-pyrrolo[3,2-c]pyridin-6-yl)-4-(3-(piperazin-1-yl)propyl)benzamide dihydrochloride (Intermediate 538, 0.69 g, 1.44 mmol) in dimethyl sulfoxide (5 mL) was added 2-(2,6-dioxo-3-piperidyl)-4-fluoro-isoindoline-1,3-dione (CAS No: 835616-60-9, 0.40 g, 1.44 mmol) and the reaction mixture was heated at 120° C. for 12 h. The reaction mixture was concentrated in vacuo. The crude material was diluted with ice-cold water (10 mL) and stirred at room temperature for 10 min. The resultant precipitate was collected by filtration and dried in vacuo. The crude material was purified by preparative HPLC (Method B) to afford 4-(3-(4-(2-(2,6-dioxopiperidin-3-yl)-1,3-dioxoisoindolin-4-yl)piperazin-1-yl)propyl)-N-(2-((R)-1-methylpyrrolidin-2-yl)-1H-pyrrolo[3,2-c]pyridin-6-yl)benzamide (Example 130, 0.215 g, 21%) as a yellow solid. ¹H NMR (400 MHz, DMSO-d₆) δ ppm 1.76-1.92 (m, 5H), 2.01-2.08 (m, 1H), 2.12-2.16 (m, 1H), 2.17 (s, 3H), 2.20-2.31 (m, 1H), 2.32-2.41 (m, 2H), 2.46-2.49 (m, 2H), 2.54-2.63 (m, 5H), 2.65-2.75 (m, 2H), 2.82-2.94 (m, 1H), 3.10-3.18 (m, 1H), 3.27-3.32 (m, 4H), 5.06-5.11 (m, 1H), 6.39 (s, 1H), 7.33-7.36 (m, 4H), 7.68-7.72 (m, 1H), 7.99 (d, J=8.31 Hz, 2H), 8.18 (s, 1H), 8.50 (s, 1H), 10.35 (br s, 1H), 11.08 (br s, 1H), 11.36 (br s, 1H). Mass spec m/z 704.2 [M+H]⁺.

### Example 131 was Synthesised Following Scheme 128

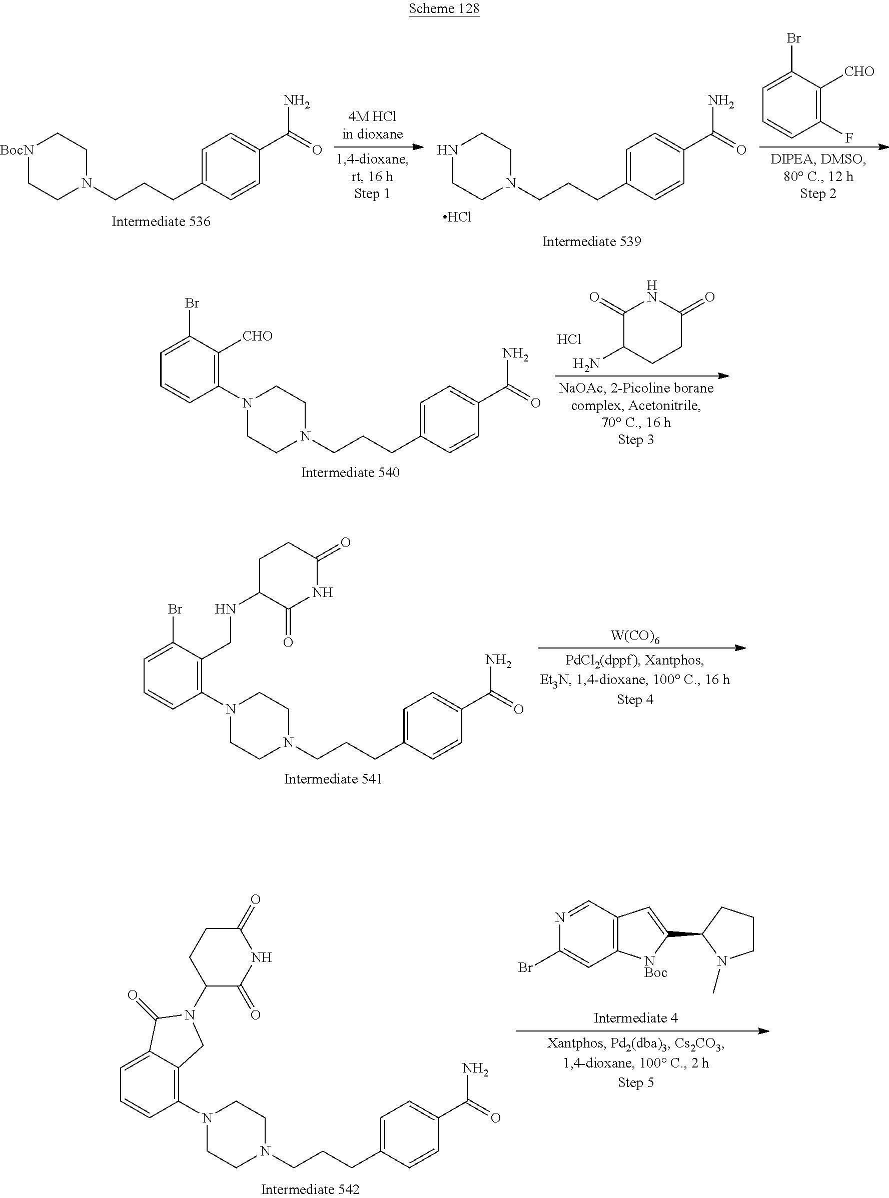

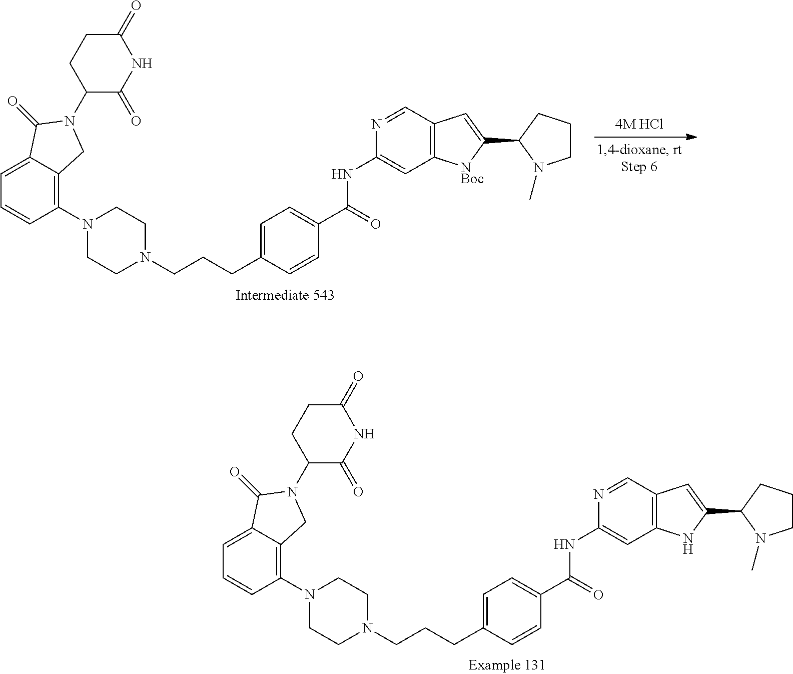 [SMILES: CN1CCC[C@@H]1c1cc2cnc(NC(=O)c3ccc(CCCN4CCN(c5cccc6c5CN(C5CCC(=O)NC5=O)C6=O)CC4)cc3)cc2n1C(=O)OC(C)(C)C ; CN1CCC[C@@H]1c1cc2cnc(NC(=O)c3ccc(CCCN4CCN(c5cccc6c5CN(C5CCC(=O)NC5=O)C6=O)CC4)cc3)cc2[nH]1]

### Step 1

### Intermediate 539: 4-(3-(piperazin-1-yl)propyl)benzamide hydrochloride

To a cooled (0° C.) solution tert-butyl 4-(3-(4-carbamoylphenyl)propyl)piperazine-1-carboxylate (Intermediate 536, 3.0 g, 8.60 mmol) in 1,4-dioxane (30 mL) was added 4M hydrochloric acid in 1,4-dioxane (30 mL) and the reaction mixture was stirred at room temperature for 16 h. The reaction mixture was then concentrated in vacuo to afford 4-(3-(piperazin-1-yl)propyl)benzamide hydrochloride (Intermediate 539, 3.6 g) as an off-white solid, which was used for the next step without further purification. ¹H NMR (400 MHz, DMSO-d₆) δ ppm 1.26-1.32 (m, 1H), 2.02-2.09 (m, 2H), 2.68-2.72 (m, 2H), 3.10-3.16 (m, 2H), 3.28-3,35 (in, 2H), 3.36-3.52 (m, 4H), 3.67-3.71 (m, 2H) 7.32 (d, J=8.0 Hz, 3H), 7.83 (d, J=8.4 Hz, 2H), 7.95 (br s, 1H). Mass spec m/z 248.0 [M+H]⁺.

### Step 2

### Intermediate 540: 4-(3-(4-(3-bromo-2-formylphenyl)piperazin-1-yl)propyl)benzamide

To a solution of 4-(3-(piperazin-1-yl)propyl)benzamide hydrochloride (Intermediate 539, 2.50 g, 10.0 mmol) in dimethyl sulfoxide (20 mL) was added 2-bromo-6-fluorobenzaldehyde (CAS 5 No. 360575-28-6, 2.10 g, 10.0 mmol) followed by N,N-diisopropylethylamine (9.02 mL, 51.0 mmol) and the reaction mixture was heated at 80° C. for 12 h. The reaction mixture was then diluted with water (2×50 mL) and extracted with dichloromethane (50 mL). The organic layer was separated, dried over anhydrous Na₂SO₄, filtered, and concentrated in vacuo. The crude material was purified by combi-flash chromatography, by eluting with 50% ethyl acetate in heptane, to afford 4-(3-(4-(3-bromo-2-formylphenyl)piperazin-1-yl)propyl)benzamide (Intermediate 540, 1.8 g, 48%) as pale yellow solid. ¹H NMR (400 MHz, DMSO-d₆) δ ppm 1.73-1.77 (m, 2H), 2.30-2.34 (m, 2H), 2.51-2.56 (m, 4H), 2.58-2.62 (m, 2H), 2.96-2.98 (m, 4H), 5.60 (bs, 1H), 6.00 (bs, 1H), 6.93-6.96 (m, 1H), 7.19-7.19 (m, 4H), 7.61-7.64 (m, 2H), 10.10 (s, 1H). Mass spec m/z 430.0 [M+H]⁺.

### Step 3

### Intermediate 541: 4-(3-(4-(3-bromo-2-(((2,6-dioxopiperidin-3-yl)amino)methyl)phenyl)piperazin-1-yl)propyl)benzamide

To a solution of 4-(3-(4-(3-bromo-2-formylphenyl)piperazin-1-yl)propyl)benzamide (Intermediate 540, 1.20 g, 2.80 mmol) and 3-aminopiperidine-2,6-dione hydrochloride (CAS No. 24666-56-6, 0.55 g, 3.30 mmol) in acetonitrile (50 mL) was added sodium acetate (0.35 g, 4.20 mmol) and 2-picoline borane complex (0.78 g, 7.0 mmol) and the reaction mixture was heated at 70° C. for 16 h. The reaction mixture was then concentrated in vacuo and the crude material purified by combi-flash chromatography, by eluting with 60% ethyl acetate in heptane, to afford 4-(3-(4-(3-bromo-2-(((2,6-dioxopiperidin-3-yl)amino)methyl)phenyl)piperazin-1-yl)propyl)benzamide (Intermediate 541, 0.43 g, 28%) as pale yellow solid. ¹H NMR (400 MHz, DMSO-d₆) δ ppm 1.90-1.92 (m, 2H), 2.27-2.36 (m, 4H), 2.53-2.57 (m, 4H), 2.64-2.70 (m, 3H), 2.80-2.84 (m, 2H), 2.97-3.11 (m, 3H), 3.22-3.28 (m, 2H), 3.86-3.97 (m, 2H), 7.17-7.35 (m, 7H), 7.78-7.89 (m, 2H), 10.10 (s, 1H). Mass spec m/z 544.2 [M+H+2]⁺.

### Step 3

### Intermediate 542: 4-(3-(4-(2-(2,6-dioxopiperidin-3-yl)-1-oxoisoindolin-4-yl)piperazin-1-yl)propyl)benzamide

To a solution of 4-(3-(4-(3-bromo-2-(((2,6-dioxopiperidin-3-yl)amino)methyl)phenyl)piperazin-1-yl)propyl)benzamide (Intermediate 541, 0.40 g, 0.73 mmol) in 1,4-dioxane (10 mL) was added triethylamine (0.31 mL, 2.21 mmol). The reaction mixture was purged with argon for 15 min then W(CO)₆ (0.778 g, 2.21 mmol) followed by PdCl₂(dppf) (0.11 g, 0.14 mmol) and Xantphos (0.08 g, 0.14 mmol) were added. The reaction mixture was further purged with argon for 10 min and heated at 100° C. for 16 h. The reaction mixture was concentrated in vacuo and the crude material purified by combi-flash chromatography, by eluting with 50% ethyl acetate in heptane, to afford 4-(3-(4-(2-(2,6-dioxopiperidin-3-yl)-1-oxoisoindolin-4-yl)piperazin-1-yl)propyl)benzamide (Intermediate 542, 0.21 g, 58%) as a pale brown solid. Mass spec m/z 490.5 [M+H]⁺.

### Step 5

### Intermediate 543: tert-butyl 6-(4-(3-(4-(2-(2,6-dioxopiperidin-3-yl)-1-oxoisoindolin-4-yl)piperazin-1-yl)propyl)benzamido)-2-((R)-1-methylpyrrolidin-2-yl)-1H-pyrrolo[3,2-c]pyridine-1-carboxylate

To a solution of 4-(3-(4-(2-(2,6-dioxopiperidin-3-yl)-1-oxoisoindolin-4-yl)piperazin-1-yl)propyl)benzamide (Intermediate 542, 0.25 g, 0.51 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.23 g, 0.61 mmol) followed by cesium carbonate (0.49 g, 1.53 mmol). The reaction mixture was purged with argon for 15 min and then Pd₂(dba)₃ (0.09 g, 0.10 mmol) and Xantphos (0.11 g, 0.20 mmol) were added. The reaction mixture was further purged with argon for another 10 min and heated at 100° C. for 2 h. The reaction mixture was then concentrated in vacuo and the crude material purified by combi-flash chromatography, by eluting with 80% ethyl acetate in heptane, to afford tert-butyl 6-(4-(3-(4-(2-(2,6-dioxopiperidin-3-yl)-1-oxoisoindolin-4-yl)piperazin-1-yl)propyl)benzamido)-2-((R)-1-methylpyrrolidin-2-yl)-1H-pyrrolo[3,2-c]pyridine-1-carboxylate (Intermediate 543, 0.20 g, 56%) as a yellow solid. Mass spec m/z 789.4 [M+H]⁺.

### Step 6

### Example 131: 4-(3-(4-(2-(2,6-dioxopiperidin-3-yl)-1-oxoisoindolin-4-yl)piperazin-1-yl)propyl)-N-(2-((R)-1-methylpyrrolidin-2-yl)-1H-pyrrolo[3,2-c]pyridin-6-yl)benzamide

To a cooled (0° C.) solution tert-butyl 6-(4-(3-(4-(2-(2,6-dioxopiperidin-3-yl)-1-oxoisoindolin-4-yl)piperazin-1-yl)propyl)benzamido)-2-((R)-1-methylpyrrolidin-2-yl)-1H-pyrrolo[3,2-c]pyridine-1-carboxylate (Intermediate 543, 0.20 g, 0.25 mmol) in 1,4-dioxane (2 mL) was added 4M hydrochloric acid in 1,4-dioxane (2 mL) and the reaction mixture was stirred at room temperature for 16 h. The reaction mixture was concentrated in vacuo and the crude material was purified by preparative HPLC (Method A) to afford 4-(3-(4-(2-(2,6-dioxopiperidin-3-yl)-1-oxoisoindolin-4-yl)piperazin-1-yl)propyl)-N-(2-((R)-1-methylpyrrolidin-2-yl)-1H-pyrrolo[3,2-c]pyridin-6-yl)benzamide (Example 131, 0.043 g, 25%) as an off-white solid. ¹H NMR (400 MHz, DMSO-d₆) δ ppm 1.23-1.94 (m, 5H), 1.95-2.02 (m, 1H), 2.13-2.28 (m, 4H), 2.23-2.30 (m, 1H), 2.32-2.40 (m, 2H), 2.41-2.43 (m, 1H), 2.52-2.61 (m, 6H), 2.67-2.73 (m, 2H), 2.84-2.94 (m, 1H), 3.02-3.18 (m, 5H), 4.27-4.33 (m, 1H), 4.42-4.46 (m, 1H), 5.09-5.14 (m, 1H), 6.38 (s, 1H), 7.16 (d, J=7.6 Hz, 1H), 7.30-7.36 (m, 3H), 7.44 (t, J=8.4 Hz, 1H), 7.98 (d, J=8.4 Hz, 2H), 8.18 (s, 1H), 8.49 (s, 1H), 10.35 (s, 1H), 10.98 (s, 1H), 11.37 (s, 1H). Mass spec m/z 689.3 [M+H]⁺.

### Example 132 was Prepared Following Scheme 129

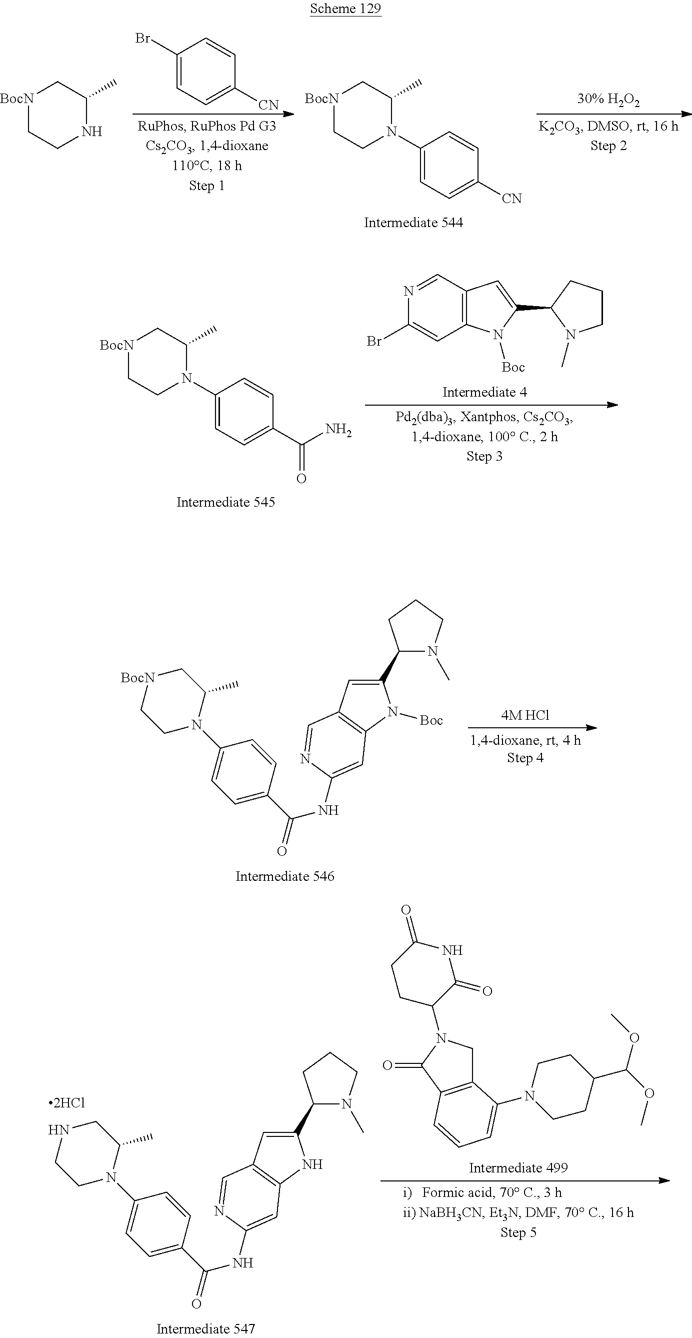

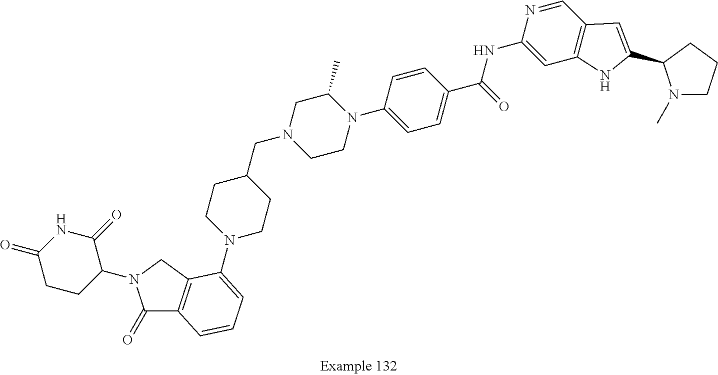 [SMILES: C[C@H]1CN(CC2CCN(c3cccc4c3CN(C3CCC(=O)NC3=O)C4=O)CC2)CCN1c1ccc(C(=O)Nc2cc3[nH]c([C@H]4CCCN4C)cc3cn2)cc1]

### Step 1

### Intermediate 544: tert-butyl (S)-4-(4-cyanophenyl)-3-methylpiperazine-1-carboxylate

To a solution of tert-butyl (S)-3-methylpiperazine-1-carboxylate (CAS No. 147081-29-6, 6.05 g, 30.2 mmol) in 1,4-dioxane (25 mL) was added 4-bromobenzonitrile (CAS No. 623-00-7, 5.0 g, 27.5 mmol) and cesium carbonate (26.9 g, 82.4 mmol). The reaction mixture was purged with argon for 15 min. Then RuPhos (1.96 g, 4.12 mmol) and RuPhos Pd G3 (2.41 g, 0.20 mmol) were added. The reaction mixture was purged with argon for another 10 min and heated 110° C. for 8 h. The reaction mixture was filtered through a celite bed, the filter pad was washed with ethyl acetate (100 mL) and the filtrate was concentrated in vacuo. The crude material was purified by combi-flash chromatography, by eluting with 25% ethyl acetate in heptane, to afford tert-butyl (S)-4-(4-cyanophenyl)-3-methylpiperazine-1-carboxylate (Intermediate 544, 4.50 g, 54%) as a brown solid. ¹H NMR (400 MHz, DMSO-d₆) δ ppm 1.99 (d, J=6.4 Hz, 3H), 1.42 (s, 9H), 2.98-3.18 (m, 3H), 3.57-3.60 (m, 1H), 3.76-3.79 (m, 1H), 3.94 (bs, 1H), 4.18 (s, 1H), 6.96 (d, J=9.2 Hz, 2H), 7.59 (d, J=9.2 Hz, 2H). Mass spec m/z 302.1 [M+H]⁺.

### Step 2

### Intermediate 545: tert-butyl (S)-4-(4-carbamoylphenyl)-3-methylpiperazine-1-carboxylate

To solution of tert-butyl (S)-4-(4-cyanophenyl)-3-methylpiperazine-1-carboxylate (Intermediate 544, 4.50 g, 14.9 mmol) in dimethyl sulfoxide (15 mL) were added potassium carbonate (3.10 g, 22.4 mmol) and hydrogen peroxide (4.49 mL, 44.8 mmol) and the reaction mixture was stirred at room temperature for 16 h. The reaction mixture was diluted with water (200 mL), the resultant precipitate was collected by filtration, washed with water (20 mL) and dried in vacuo to afford tert-butyl (S)-4-(4-carbamoylphenyl)-3-methylpiperazine-1-carboxylate (Intermediate 545, 3.5 g, 73%) as yellow solid. ¹H NMR (400 MHz, DMSO-d₆) δ ppm 0.94-0.96 (m, 3H), 1.42 (s, 9H), 2.95-3.08 (m, 2H), 3.15-3.21 (m, 1H), 3.45-3.47 (m, 1H), 3.76-3.79 (m, 1H), 3.91-3.99 (m, 1H), 4.14 (bs, 1H), 6.87-6.89 (m, 2H), 6.99 (bs, 1H), 7.68 (bs, 1H), 7.73-7.75 (m, 2H). Mass spec m/z 320.1 [M+H]⁺.

### Step 3

### Intermediate 546: tert-butyl 6-(4-((S)-4-(tert-butoxycarbonyl)-2-methylpiperazin-1-yl)benzamido)-2-((R)-1-methylpyrrolidin-2-yl)-1H-pyrrolo[3,2-c]pyridine-1-carboxylate

To a solution of tert-butyl (S)-4-(4-carbamoylphenyl)-3-methylpiperazine-1-carboxylate (Intermediate 545, 1.0 g, 3.13 mmol) in 1,4-dioxane (10 mL) were added tert-butyl (R)-6-bromo-2-(1-methylpyrrolidin-2-yl)-1H-pyrrolo[3,2-c]pyridine-1-carboxylate (Intermediate 4, 1.31 g, 3.44 mmol) and cesium carbonate (3.6 g, 9.39 mmol). The reaction mixture was purged with argon for 15 min, then Xanthphos (0.56 g, 0.94 mmol) and Pd₂(dba)₃ (0.44 g, 0.47 mmol) were added. The reaction was was purged with argon for another 10 min and heated at 100° C. for 2 h. The reaction mixture was filleted through a celite bed, the filter pad was washed with ethyl acetate (100 mL) and the filtrate was concentrated in vacuo. The crude material was purified by combi-flash chromatography, by eluting with 15% MeOH in DCM, to afford tert-butyl 6-(4-((S)-4-(tert-butoxycarbonyl)-2-methylpiperazin-1-yl)benzamido)-2-((R)-1-methylpyrrolidin-2-yl)-1H-pyrrolo[3,2-c]pyridine-1-carboxylate (Intermediate 546, 1.5 g, 77%) as a brown solid. ¹H NMR (400 MHz, DMSO-d₆) δ ppm 0.98-1.00 (m, 3H), 1.43 (s, 9H), 1.60-1.75 (m, 12H), 2.30-2.39 (m, 5H), 2.98-3.35 (m, 4H), 3.53-3.55 (m, 1H), 3.79-3.82 (m, 1H), 3.90-3.98 (m, 2H), 4.16-4.20 (m, 1H), 6.74 (s, 1H), 6.93 (d, J=8.4 Hz, 2H), 7.98 (d, J=8.4 Hz, 2H), 8.56 (s, 1H), 8.92 (s, 1H), 10.3 (m, 1H). Mass spec m/z 619.3 [M+H]⁺.

### Step 4

### Intermediate 547: 4-((S)-2-methylpiperazin-1-yl)-N-(2-((R)-1-methylpyrrolidin-2-yl)-1H-pyrrolo[3,2-c]pyridin-6-yl)benzamide hydrochloride

To a cooled (0° C.) solution of tert-butyl 6-(4-((S)-4-(tert-butoxycarbonyl)-2-methylpiperazin-1-yl)benzamido)-2-((R)-1-methylpyrrolidin-2-yl)-1H-pyrrolo[3,2-c]pyridine-1-carboxylate (Intermediate 546, 1.5 g, 2.4 mmol) in 1,4-dioxane (10 mL) was added 4M hydrochloric acid in 1,4-dioxane (10 mL) and the reaction mixture was stirred at room temperature for 4 h. The reaction mixture was concentrated in vacuo to afford 4-((S)-2-methylpiperazin-1-yl)-N-(2-((R)-1-methylpyrrolidin-2-yl)-1H-pyrrolo[3,2-c]pyridin-6-yl)benzamide hydrochloride (Intermediate 547, 1.5 g) as yellow gummy solid, which was used for next step without further purification. ¹H NMR (400 MHz, DMSO-d₆) δ ppm 1.23 (d, J=7.4 Hz, 3H), 2.15-2.23 (m, 2H), 2.31-2.46 (m, 2H), 2.52-2.58 (m, 1H), 2.80-2.82 (m, 2H), 3.00-3.06 (m, 1H), 3.20-3.41 (m, 6H), 3.68-3.78 (m, 1H), 3.85-3.89 (m, 1H), 4.48-4.51 (m, 1H), 4.72-4.82 (m, 1H) 7.09 (d, J=9.2 Hz, 2H), 7.29 (s, 1H), 8.17 (d, J=9.2 Hz, 2H), 8.30 (s, 1H), 9.08 (s, 1H), 11.94 (s, 1H), 13.40 (s, 1H). Mass spec m/z 419.3 [M+H]⁺.

### Step 5

### Example 132: 4-((2S)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide

A solution of 3-(4-(4-(dimethoxymethyl)piperidin-1-yl)-1-oxoisoindolin-2-yl)piperidine-2,6-dione (Intermediate 499, 0.56 g, 1.24 mmol) in formic acid (4 mL) was heated at 70° C. for 3 h. The reaction mixture was concentrated in vacuo, then dimethylformamide (3.0 mL), 4-((S)-2-methylpiperazin-1-yl)-N-(2-((R)-1-methylpyrrolidin-2-yl)-1H-pyrrolo[3,2-c]pyridin-6-yl)benzamide dihydrochloride (Intermediate 547, 0.57 g, 1.25 mmol), sodium cyanoborohydride (0.33 g, 4.98 mmol) and triethylamine (0.87 mL, 6.23 mmol) were added. The reaction mixture was stirred at 70° C. for 16 h then diluted with ice cold water (200 mL). The resultant precipitate was collected by filtration and dried in vacuo. The crude material was purified by preparative HPLC (Method A) to afford 4-((2S)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide (Example 132, 0.17 g, 18%) as an off-white solid. ¹H NMR (400 MHz, DMSO-d₆) δ ppm 1.11 (d, J=6.4 Hz, 3H), 1.34-1.46 (m, 2H), 1.69-1.93 (m, 6H), 1.95-2.23 (m, 10H), 2.52-2.63 (m, 2H), 2.71-2.81 (m, 3H), 2.88-2.97 (m, 2H), 2.98-3.06 (m, 1H), 3.12-3.17 (m, 1H), 3.28-3.30 (m, 1H), 3.36-3.46 (m, 2H), 3.52-3.56 (m, 1H), 4.19-4.21 (m, 1H), 4.28-4.32 (m, 1H), 4.42-4.46 (m, 1H), 5.10-5.14 (m, 1H), 6.37 (s, 1H), 6.91 (d, J=8.8 Hz, 2H), 7.16 (d, J=8.0 Hz, 1H), 7.29 (d, J=8.8 Hz, 1H), 7.43 (t, J=8.0 Hz, 1H), 7.95 (d, J=8.8 Hz, 2H), 8.17 (s, 1H), 8.47 (s, 1H), 10.05 (s, 1H), 10.98 (s, 1H), 11.32 (s, 1H). Mass spec m/z 758.3 [M+H]⁺.

### Example 133 was Prepared Following Scheme 130

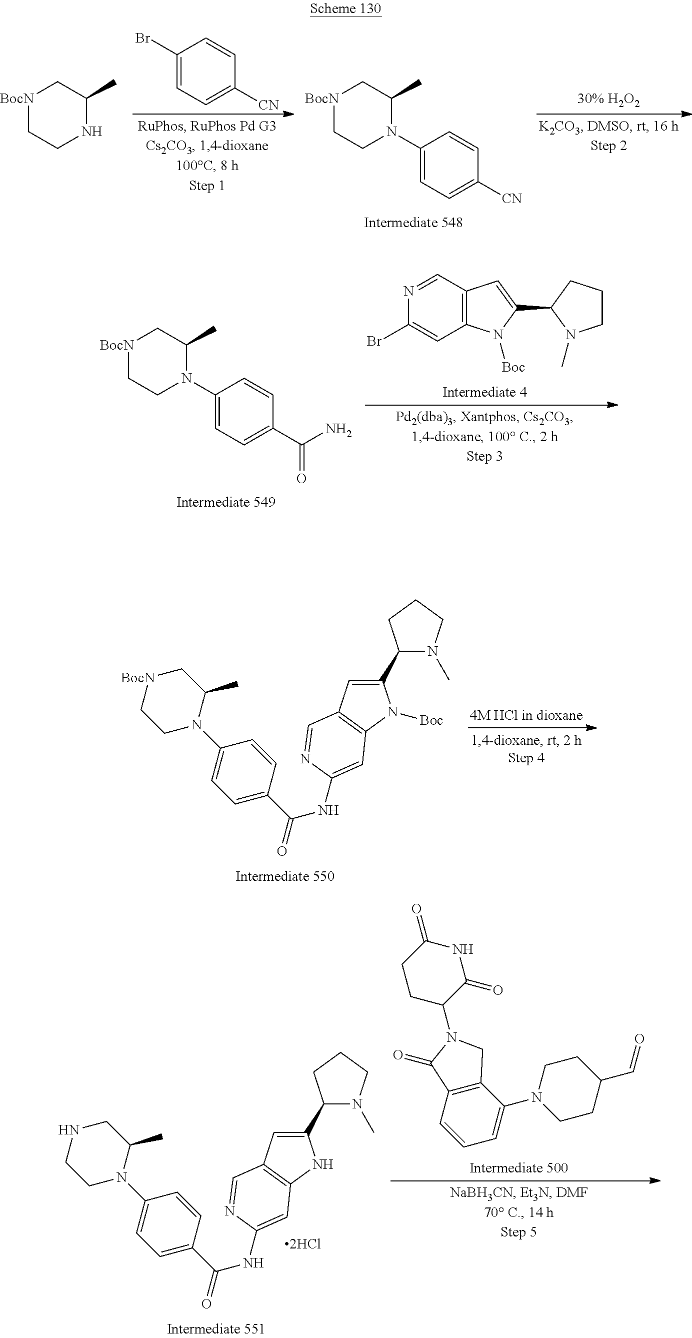

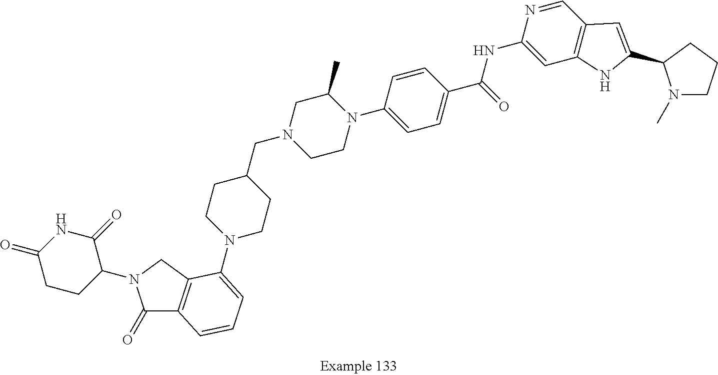 [SMILES: C[C@@H]1CN(CC2CCN(c3cccc4c3CN(C3CCC(=O)NC3=O)C4=O)CC2)CCN1c1ccc(C(=O)Nc2cc3[nH]c([C@H]4CCCN4C)cc3cn2)cc1]

### Step 1

### Intermediate 548: tert-butyl (R)-4-(4-cyanophenyl)-3-methylpiperazine-1-carboxylate

To a solution of tert-butyl (R)-3-methylpiperazine-1-carboxylate (CAS No. 163765-44-4, 6.0 g, 29.7 mmol) in 1,4-dioxane (50 mL) was added 4-bromobenzonitrile (CAS No. 210-764-9, 6.0 g, 32.6 mmol) followed by cesium carbonate (29.0 g, 89.0 mmol). The reaction mixture was purged with argon for 15 min then RuPhos (2.82 g, 5.93 mmol) and RuPhos Pd G3 (2.61 g, 2.97 mmol) were added. The reaction mixture was further purged with argon for 10 min and stirred at 100° C. for 8 h then concentrated in vacuo. The crude material was purified by combi-flash chromatography, by eluting with 20% ethyl acetate in heptane, to afford tert-butyl (R)-4-(4-cyanophenyl)-3-methylpiperazine-1-carboxylate (Intermediate 548, 6.0 g, 67%) as an off-white solid. ¹H NMR (400 MHz, DMSO-d₆) δ ppm 0.95-1.04 (m, 3H), 1.41 (s, 9H), 2.95-3.09 (m, 2H), 3.11-3.25 (m, 1H), 3.53-3.63 (m, 1H), 3.73-3.82 (m, 1H), 3.87-3.99 (m, 1H), 4.12-4.23 (m, 1H), 6.96 (d, J=8.69 Hz, 2H), 7.58 (d, J=8.31 Hz, 2H). Mass spec m/z 302.1 [M+H]⁺.

### Step 2

### Intermediate 549: tert-butyl (R)-4-(4-carbamoylphenyl)-3-methylpiperazine-1-carboxylate

To a cooled (0° C.) solution of tert-butyl (R)-4-(4-cyanophenyl)-3-methylpiperazine-1-carboxylate (Intermediate 548, 4.0 g, 13.3 mmol) in dimethyl sulfoxide (20 mL) was added potassium carbonate (4.58 g, 33.2 mmol) and 30% hydrogen peroxide (10.4 mL, 133.0 mmol) and the reaction mixture was stirred at room temperature for 16 h. The reaction mixture was diluted with water (100 mL), the resultant precipitate was collected by filtration, washed with water (50 mL), and dried in vacuo to afford tert-butyl (R)-4-(4-carbamoylphenyl)-3-methylpiperazine-1-carboxylate (Intermediate 549, 3.5 g, 83%) as an off-white solid, which was used for the next step without further purification. ¹H NMR (400 MHz, DMSO-d₆) δ ppm 0.91-0.98 (m, 3H), 1.42 (s, 9H), 2.52-2.57 (m, 2H), 2.93-3.03 (m, 1H), 3.12-3.21 (m, 1H), 3.42-3.51 (m, 1H), 3.74-3.83 (m, 1H), 3.90-3.41 (m, 1H), 6.88 (d, J=8.22 Hz, 2H), 7.01 (br s, 1H), 7.69 (br s, 1H), 7.75 (d, J=7.83 Hz, 2H). Mass spec m/z 320.3 [M+H]⁺.

### Step 3

### Intermediate 550: tert-butyl 6-(4-((R)-4-(tert-butoxycarbonyl)-2-methylpiperazin-1-yl)benzamido)-2-((R)-1-methylpyrrolidin-2-yl)-1H-pyrrolo[3,2-c]pyridine-1-carboxylate

To a solution of tert-butyl (R)-4-(4-carbamoylphenyl)-3-methylpiperazine-1-carboxylate (Intermediate 549, 1.5 g, 4.7 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 2.0 g, 5.2 mmol) followed by cesium carbonate (4.6 g, 14.0 mmol). The reaction mixture was purged with argon for 15 min then Pd₂(dba)₃ (0.67 g, 0.70 mmol) and Xantphos (0.84 g, 1.4 mmol) were added. The reaction mixture was further purged with argon for 10 min and stirred at 100° C. for 2 h. The reaction mixture was concentrated in vacuo and the crude material purified by combi-flash chromatography, by eluting with 80% ethyl acetate in heptane, to afford tert-butyl 6-(4-((R)-4-(tert-butoxycarbonyl)-2-methylpiperazin-1-yl)benzamido)-2-((R)-1-methylpyrrolidin-2-yl)-1H-pyrrolo[3,2-c]pyridine-1-carboxylate (Intermediate 550, 1.8 g, 62%) as a pale yellow solid. ¹H NMR (400 MHz, DMSO-d₆) δ ppm 0.93-1.10 (m, 3H), 1.42 (s, 9H), 1.59-1.64 (m, 2H), 1.68 (s, 9H), 1.61-1.64 (m, 1H), 2.35 (s, 3H), 2.36-2.41 (m, 2H), 2.95-3.08 (m, 1H), 3.10-3.25 (m, 2H), 3.50-3.60 (m, 1H), 3.72-3.82 (m, 1H), 3.88-4.07 (m, 2H), 4.11-4.22 (m, 1H), 5.75 (s, 1H), 6.73 (s, 1H), 6.88-6.98 (m, 2H), 7.94-7.97 (d, J=8.69 Hz, 2H), 8.56 (s, 1H), 8.91 (s, 1H), 10.31 (br s, 1H). Mass spec m/z 619.3 [M+H]⁺.

### Step 4

### Intermediate 551: 4-((R)-2-methylpiperazin-1-yl)-N-(2-((R)-1-methylpyrrolidin-2-yl)-1H-pyrrolo[3,2-c]pyridin-6-yl)benzamide dihydrochloride

To a cooled (0° C.) solution of tert-butyl 6-(4-((R)-4-(tert-butoxycarbonyl)-2-methylpiperazin-1-yl)benzamido)-2-((R)-1-methylpyrrolidin-2-yl)-1H-pyrrolo[3,2-c]pyridine-1-carboxylate (Intermediate 550, 0.90 g, 1.45 mmol) in dichloromethane (6 mL) was added 4M hydrochloric acid in 1,4-dioxane (9 mL, 36 mmol). The reaction mixture was stirred at room temperature for 2 h then concentrated in vacuo. The crude material was triturated with diethyl ether (2×10 mL) and dried in vacuo to afford 4-((R)-2-methylpiperazin-1-yl)-N-(2-((R)-1-methylpyrrolidin-2-yl)-1H-pyrrolo[3,2-c]pyridin-6-yl)benzamide dihydrochloride (Intermediate 551, 0.80 g, 80%) as a white solid. Mass spec m/z 419.1 [M+H]⁺.

### Example 133: 4-((2R)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide

To a solution of 4-((R)-2-methylpiperazin-1-yl)-N-(2-((R)-1-methylpyrrolidin-2-yl)-1H-pyrrolo[3,2-c]pyridin-6-yl)benzamide dihydrochloride (Intermediate 551, 0.50 g, 1.09 mmol) and 1-(2-(2,6-dioxopiperidin-3-yl)-1-oxoisoindolin-4-yl)piperidine-4-carbaldehyde (Intermediate 500, 0.51 g, 1.09 mmol) in dimethylformamide (5 mL) was added triethylamine (0.44 g, 4.39 mmol). The reaction mixture was stirred at 70° C. for 2 h. The reaction mixture was cooled to room temperature and then sodium cyanoborohydride (0.21 g, 3.29 mmol) was added. The reaction mixture was stirred at 70° C. for 12 h. and then poured in ice cold water (30 mL). The resultant precipitate was collected by filtration and dried in vacuo. The crude material was purified by preparative HPLC (Method A) to afford 4-((2R)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide (Example 133, 0.11 g, 14%) as an off-white solid. ¹H NMR (400 MHz, DMSO-d₆) δ ppm 1.09-1.14 (m, 3H), 1.24-1.35 (m, 2H), 1.74-2.03 (m, 7H), 2.13-2.16 (m, 6H), 2.28 (s, 3H), 2.56-2.63 (m, 2H), 2.74-2.82 (m, 3H), 2.87-2.97 (m, 2H), 2.98-3.08 (m, 2H), 3.10-3.18 (m, 1H), 3.37-3.46 (m, 2H), 3.47-3.59 (m, 1H), 4.17-4.24 (m, 1H), 4.26-4.33 (m, 1H), 4.39-4.49 (m, 1H), 5.09-5.14 (m, 1H), 6.37 (s, 1H), 6.92 (d, J=8.85 Hz, 2H), 7.16 (d, J=7.64 Hz, 1H), 7.29 (d, J=7.64 Hz, 1H), 7.39-7.46 (m, 1H), 7.96 (d, J=8.85 Hz, 2H), 8.17 (s, 1H), 8.47 (s, 1H), 10.04 (br s, 1H), 10.98 (br s, 1H), 11.31 (br s, 1H). Mass spec m/z 758.3 [M+H]⁺.

### Examples 134 and 135 were synthesised following Scheme 131

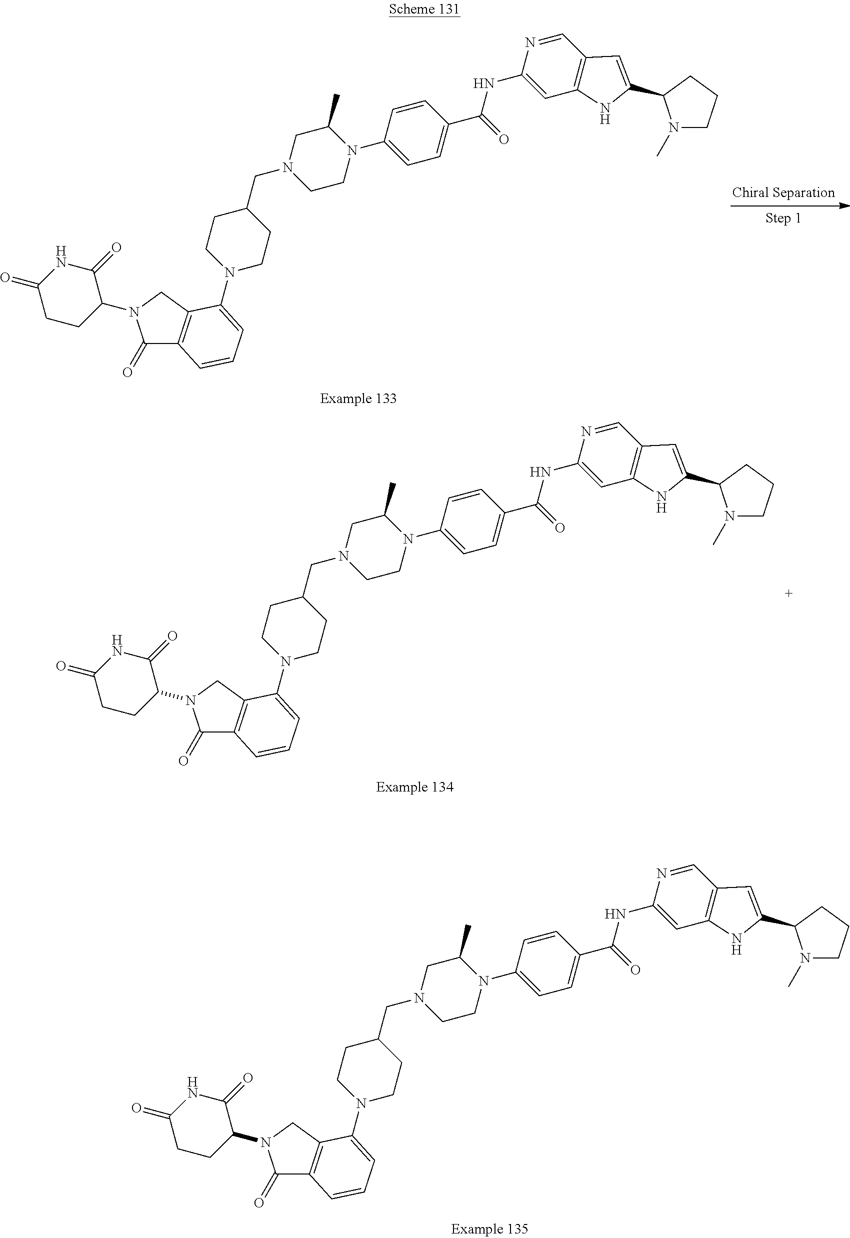

### Step 1

### Example 134: 4-((R)-4-((1-(2-((rel-R)-2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide and Example 135: 4-((R)-4-((1-(2-((rel-S)-2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide

4-((2R)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide (Example 133, 0.09 g, 0.11 mmol) was separated by chiral preparative HPLC (Column: 1H (30×250 mm), 5 m; Mobile Phase A: N-hexane, Mobile Phase B: DCM:iPrOH (1:1); Flow rate: 56 ml/min; isocratic 40% B) to afford 4-((R)-4-((1-(2-((rel-R)-2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide (Example 134, 1st eluting peak, 0.02 g, 17%) as an off-white solid and 4-((R)-4-((1-(2-((rel-S)-2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide (Example 135, 2nd eluting peak, 0.02 g, 17%) as an off-white solid

### Example 134: 4-((R)-4-((1-(2-((rel-R)-2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide

1^{st} eluting peak from Chiral preparative HPLC. Retention time: 6.02 mins.

¹H NMR (400 MHz, DMSO-d₆) δ ppm 1.09-1.13 (m, 3H), 1.29-1.35 (m, 2H), 1.77-1.98 (m, 6H), 1.94-2.03 (m, 1H), 2.05-2.14 (m, 1H), 2.16 (s, 3H), 2.21-2.28 (m, 3H), 2.59-2.64 (m, 1H), 2.75-2.81 (m, 4H), 2.87-3.02 (m, 4H), 3.11-3.14 (m, 1H), 3.40-3.42 (m, 2H), 3.49-3.58 (m, 2H), 4.17-4.23 (m, 1H), 4.26-4.33 (m, 1H), 4.40-4.47 (m, 1H), 4.53-4.56 (m, 1H), 5.09-5.14 (m, 1H), 6.37 (s, 1H), 6.92 (d, J=9.13 Hz, 2H), 7.17 (d, J=8.00 Hz, 1H), 7.29 (d, J=7.38 Hz, 1H), 7.41-7.44 (m, 1H), 7.96 (d, J=8.88 Hz, 2H), 8.17 (s, 1H), 8.47 (s, 1H), 10.04 (br s, 1H), 10.98 (br s, 1H), 11.31 (br s, 1H). Mass spec m/z 758.2 [M+H]⁺.

### Example 135: 4-((R)-4-((1-(2-((rel-S)-2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide

2nd eluting peak from Chiral preparative HPLC. Retention time: 7.81 mins.

¹H NMR (400 MHz, DMSO-d₆) δ ppm 1.08-1.12 (m, 3H), 1.25-1.38 (m, 3H), 1.70-1.82 (m, 2H), 1.83-1.93 (m, 4H), 1.95-2.04 (m, 1H), 2.04-2.15 (m, 2H), 2.16 (s, 3H), 2.17-2.19 (m, 1H), 2.19-2.30 (m, 4H), 2.59-2.64 (m, 1H), 2.76-2.84 (m, 2H), 2.87-2.98 (m, 3H), 2.98-3.07 (m, 1H), 3.11-3.17 (m, 1H), 3.37-3.44 (m, 2H), 3.51-3.58 (m, 1H), 4.16-4.24 (m, 1H), 4.27-4.33 (m, 1H), 4.40-4.48 (m, 1H), 5.08-5.16 (m, 1H), 6.37 (s, 1H), 6.92 (d, J=9.13 Hz, 2H), 7.17 (d, J=7.88 Hz, 1H), 7.29 (d, J=7.25 Hz, 1H), 7.39-7.46 (m, 1H), 7.96 (d, J=9.01 Hz, 2H), 8.17 (s, 1H), 8.47 (s, 1H), 10.05 (s, 1H), 10.98 (s, 1H), 11.31 (s, 1H). Mass spec m/z 758.3 [M+H]⁺.

### Example 136 was Synthesised Following Scheme 132

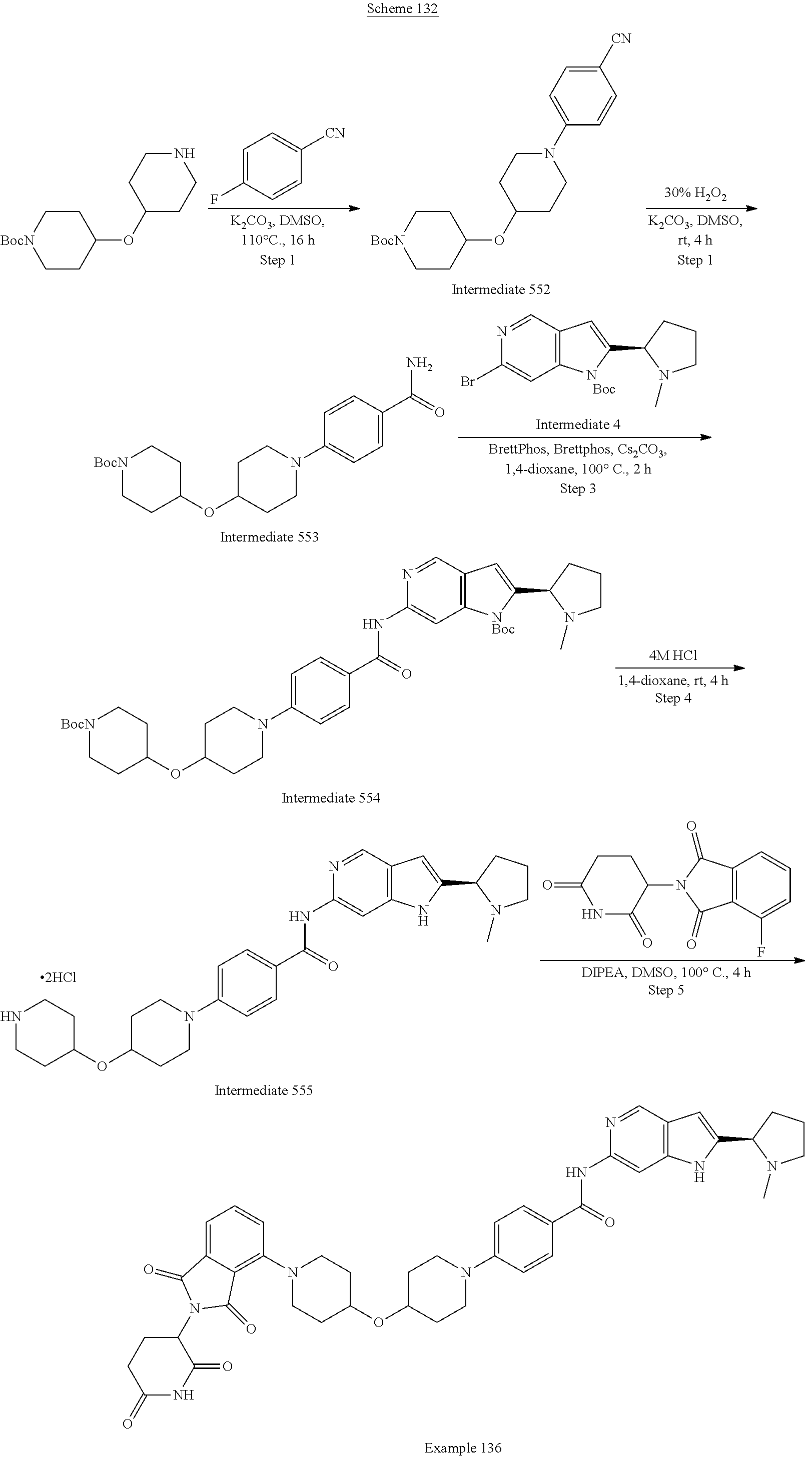

### Step 1

### Intermediate 552: tert-butyl 4-((1-(4-cyanophenyl)piperidin-4-yl)oxy)piperidine-1-carboxylate

To a solution of 4-fluorobenzonitrile (CAS No: 1194-02-1, 2.12 g, 17.58 mmol) in dimethyl sulfoxide (50 mL) were added tert-butyl 4-(piperidin-4-yloxy)piperidine-1-carboxylate (CAS No: 845305-83-1, 5.0 g, 17.58 mmol) followed by potassium carbonate (12.15 g, 87.90 mmol) and the reaction mixture was heated at 110° C. for 16 h. The reaction mixture was poured into ice cold water (100 mL), the resultant precipitate was collected by filtration and dried in vacuo. The crude material was purified by combi-flash chromatography, by eluting with 2% ethyl acetate in heptane, to afford of tert-butyl 4-((1-(4-cyanophenyl)piperidin-4-yl)oxy)piperidine-1-carboxylate (Intermediate 552, 1.5 g, 22%) as an off-white solid. ¹H NMR (400 MHz, DMSO-d6) δ ppm 1.20-1.35 (m, 2H), 1.35 (s, 9H), 1.42-1.48 (m, 2H), 1.69-1.78 (m, 2H), 1.81-1.1.87 (m, 2H), 2.95-3.04 (m, 2H), 3.08-3.12 (m, 2H), 3.59-3.70 (m, 6H), 6.92-7.06 (m, 2H), 7.50-7.60 (m, 2H). Mass spec m/z 386.2 [M+H]⁺.

### Step 2

### Intermediate 553: tert-butyl 4-((1-(4-carbamoylphenyl)piperidin-4-yl)oxy)piperidine-1-carboxylate

To a cooled (0° C.) solution of tert-butyl 4-((1-(4-cyanophenyl)piperidin-4-yl)oxy)piperidine-1-carboxylate (Intermediate 552, 2.5 g, 6.50 mmol) in dimethyl sulfoxide (25 mL) was added potassium carbonate (2.63 g, 19.0 mmol) followed by 30% hydrogen peroxide (2.0 mL, 19.0 mmol) and the reaction mixture was stirred at room temperature for 4 h. After completion, the reaction mixture was poured into ice cold water (200 mL). The resultant precipitate was collected by filtration and dried in vacuo to afford tert-butyl 4-((1-(4-carbamoylphenyl)piperidin-4-yl)oxy)piperidine-1-carboxylate (Intermediate 553, 2.0 g, 76%) as a white solid. ¹H NMR (400 MHz, DMSO-d₆) δ ppm 1.30-1.34 (m, 2H), 1.39 (s, 9H), 1.42-1.49 (m, 2H), 1.73-1.78 (m, 2H), 1.82-1.88 (m, 2H), 2.53-2.56 (m, 3H), 2.95-3.09 (m, 3H), 3.59-3.69 (m, 4H), 6.91 (d, J=8.61 Hz, 1H), 6.97 (br s, 1H), 7.00 (d, J=8.61 Hz, 1H), 7.66 (br s, 1H), 7.72 (d, J=8.22 Hz, 1H), 7.93 (d, J=8.61, 1H). Mass spec m/z 404.1 [M+H]⁺.

### Step 3

### Intermediate 554: tert-butyl (R)-6-(4-(4-((1-(tert-butoxycarbonyl)piperidin-4-yl)oxy)piperidin-1-yl)benzamido)-2-(1-methylpyrrolidin-2-yl)-1H-pyrrolo[3,2-c]pyridine-1-carboxylate

To a solution of tert-butyl 4-((1-(4-carbamoylphenyl)piperidin-4-yl)oxy)piperidine-1-carboxylate (Intermediate 553, 0.8 g, 1.05 mmol) in 1,4-dioxane (6.0 mL) was added tert-butyl (R)-6-bromo-2-(1-methylpyrrolidin-2-yl)-1H-pyrrolo[3,2-c]pyridine-1-carboxylate (Intermediate 4, 0.5 g, 1.32 mmol) followed by cesium carbonate (1.28 g, 3.94 mmol). The reaction mixture was purged with argon for 15 min, followed by the addition of BrettPhos (0.21 g, 0.39 mmol) and BrettPhos Pd G3 (0.35 g, 0.39 mmol). The reaction mixture was further purged with argon for 10 min and heated at 100° C. for 2 h. After completion, the reaction mixture was filtered through celite, and the filtrate was concentrated in vacuo. The crude residue was purified through combi-flash chromatography, by eluting with 90-95% ethyl acetate in heptane, to afford tert-butyl (R)-6-(4-(4-((1-(tert-butoxycarbonyl)piperidin-4-yl)oxy)piperidin-1-yl)benzamido)-2-(1-methylpyrrolidin-2-yl)-1H-pyrrolo[3,2-c]pyridine-1-carboxylate (Intermediate 554, 0.3 g, 32%) as a brown solid. ¹H NMR (400 MHz, DMSO-d₆) δ ppm 1.30-1.33 (m, 3H), 1.39 (s, 9H), 1.49-1.50 (m, 2H), 1.59-1.64 (m, 2H), 1.69 (s, 9H), 1.71-1.79 (m, 3H), 1.85-1.92 (m, 2H), 2.29-2.33 (m, 2H), 2.35 (s, 3H), 2.53-2.59 (m, 2H), 3.01-3.14 (m, 6H), 3.62-3.71 (m, 3H), 6.73 (s, 1H), 6.97 (d, J=8.80 Hz, 2H), 7.94 (d, J=8.40 Hz, 2H), 8.60 (s, 1H), 8.91 (s, 1H), 10.30 (s, 1H). Mass spec m/z 703.1 [M+H]⁺.

### Step 4

### Intermediate 555: (R)—N-(2-(1-methylpyrrolidin-2-yl)-1H-pyrrolo[3,2-c]pyridin-6-yl)-4-(4-(piperidin-4-yloxy)piperidin-1-yl)benzamide dihydrochloride

To a solution of tert-butyl (R)-6-(4-(4-((1-(tert-butoxycarbonyl)piperidin-4-yl)oxy)piperidin-1-yl)benzamido)-2-(1-methylpyrrolidin-2-yl)-1H-pyrrolo[3,2-c]pyridine-1-carboxylate (Intermediate 554, 0.3 g, 0.43 mmol) in 1,4-dioxane (4.0 mL) was added 4M HCl in 1,4-dioxane (4.0 mL) and the reaction mixture was stirred at room temperature for 4 h. After completion, the reaction mixture was concentrated in vacuo to afford (R)—N-(2-(1-methylpyrrolidin-2-yl)-1H-pyrrolo[3,2-c]pyridin-6-yl)-4-(4-(piperidin-4-yloxy)piperidin-1-yl)benzamide dihydrochloride (Intermediate 555, 0.3 g) as a yellow solid, which was used in the next step without purification. Mass spec m/z 503.46 [M+H]⁺.

### Step 5

### Example 136: 4-(4-((1-(2-(2,6-dioxopiperidin-3-yl)-1,3-dioxoisoindolin-4-yl)piperidin-4-yl)oxy)piperidin-1-yl)-N-(2-((R)-1-methylpyrrolidin-2-yl)-1H-pyrrolo[3,2-c]pyridin-6-yl)benzamide

To a solution of (R)—N-(2-(1-methylpyrrolidin-2-yl)-1H-pyrrolo[3,2-c]pyridin-6-yl)-4-(4-(piperidin-4-yloxy)piperidin-1-yl)benzamide dihydrochloride (Intermediate 555, 0.3 g, 0.6 mmol) in dimethyl sulfoxide (4.0 mL) were added 2-(2,6-dioxopiperidin-3-yl)-4-fluoroisoindoline-1,3-dione (CAS No: 835616-60-9, 0.16 g, 0.59 mmol) followed by N,N-diisopropylethylamine (0.52 mL, 2.98 mmol). The reaction mixture was heated at 100° C. for 4 h. The reaction mixture was then quenched with ice-cold water. The resultant precipitate was collected by filtration and dried in vacuo. to afford The crude material was purified by preparative HPLC (Method A) to afford 4-(4-((1-(2-(2,6-dioxopiperidin-3-yl)-1,3-dioxoisoindolin-4-yl)piperidin-4-yl)oxy)piperidin-1-yl)-N-(2-((R)-1-methylpyrrolidin-2-yl)-1H-pyrrolo[3,2-c]pyridin-6-yl)benzamide (Example 136, 0.03 g, 7%) as an off-white solid. ¹H NMR (400 MHz, DMSO-d₆) δ ppm 1.44-1.59 (m, 2H), 1.61-1.72 (m, 2H), 1.78-2.11 (m, 9H), 2.12-2.14 (m, 1H), 2.16 (s, 3H), 2.23-2.29 (m, 1H), 2.59-2.63 (m, 2H), 2.83-2.92 (m, 1H), 3.08-3.17 (m, 5H), 3.52-3.57 (m, 2H), 3.68-3.75 (m, 4H), 5.07-6.12 (m, 1H), 6.40 (s, 1H), 7.01 (d, J=9.20 Hz, 2H), 7.32-7.40 (m, 2H), 7.68 (t, J=8.40 Hz, 1H), 7.95 (d, J=9.20 Hz, 2H) 8.17 (s, 1H), 8.50 (s, 1H), 10.10 (s, 1H), 11.31 (s, 1H). Mass spec m/z 758.9 [M+H]⁺.

### Example 137 was Synthesised Following Scheme 133

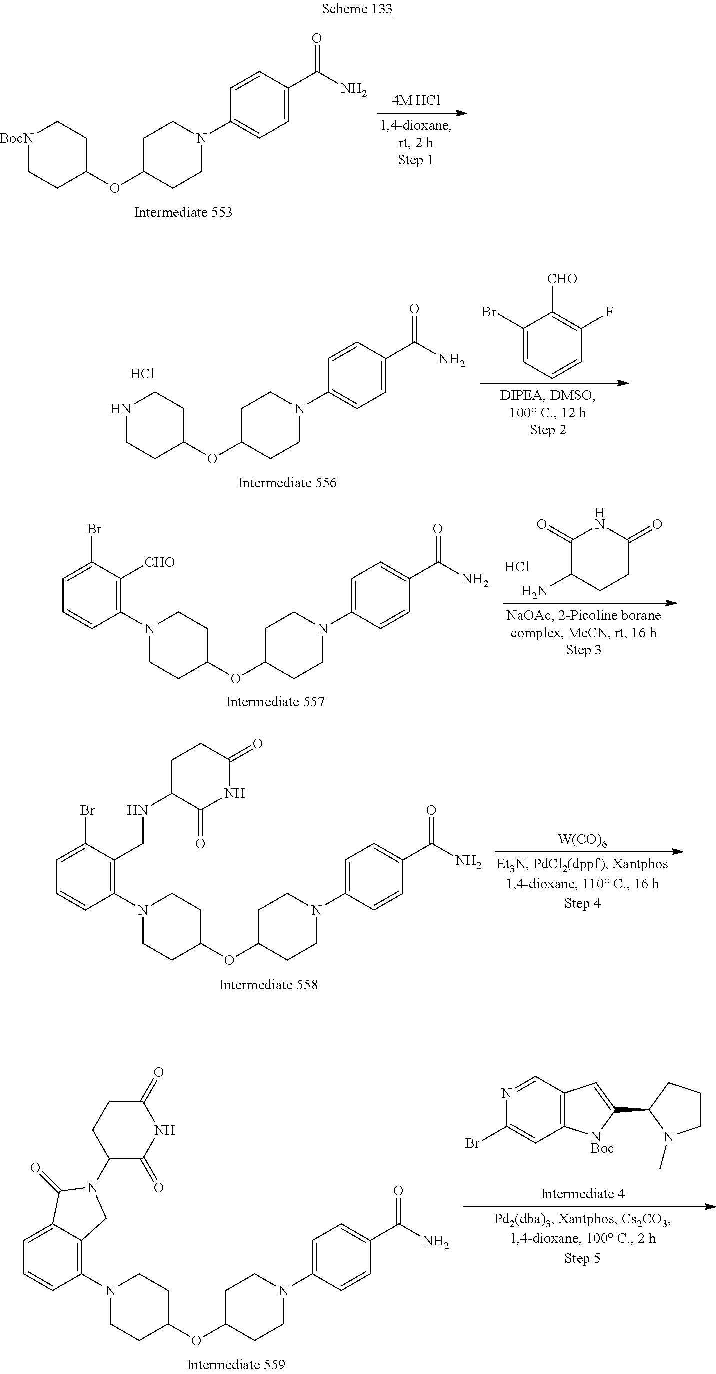

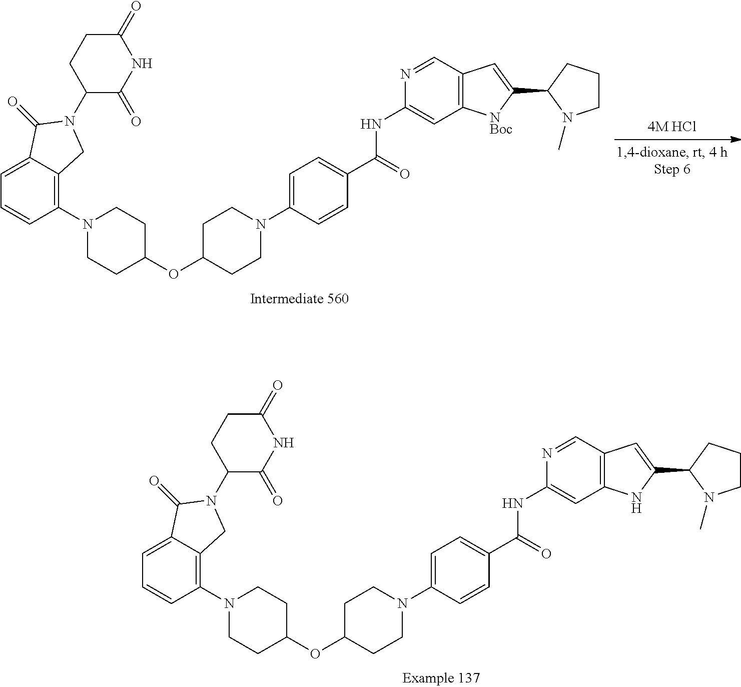 [SMILES: CN1CCC[C@@H]1c1cc2cnc(NC(=O)c3ccc(N4CCC(OC5CCN(c6cccc7c6CN(C6CCC(=O)NC6=O)C7=O)CC5)CC4)cc3)cc2n1C(=O)OC(C)(C)C ; CN1CCC[C@@H]1c1cc2cnc(NC(=O)c3ccc(N4CCC(OC5CCN(c6cccc7c6CN(C6CCC(=O)NC6=O)C7=O)CC5)CC4)cc3)cc2[nH]1]

### Step 1

### Intermediate 556: 4-(4-(piperidin-4-yloxy)piperidin-1-yl)benzamide hydrochloride

To a solution of tert-butyl 4-((1-(4-carbamoylphenyl)piperidin-4-yl)oxy)piperidine-1-carboxylate (Intermediate 553, 8.0 g, 20.8 mmol) in 1,4-dioxane (80 mL) was added 4M HCl in 1,4-dioxane (80 mL) and the reaction mixture was stirred at room temperature for 2 h. After completion, the reaction mixture was concentrated in vacuo to afford 4-(4-(piperidin-4-yloxy)piperidin-1-yl)benzamide hydrochloride (Intermediate 556, 8.0 g) as pale yellow solid, which was utilized for the next step without further purification. ¹H NMR (400 MHz, DMSO-d₆) δ ppm 1.68-1.80 (m, 4H), 1.93-2.06 (m, 4H), 2.85-2.97 (m, 3H), 3.06-3.17 (m, 2H), 3.24-3.34 (m, 2H), 3.58-3.64 (m, 2H), 3.67-3.79 (m, 2H), 7.52 (br s, 2H), 7.89 (d, J=7.93 Hz, 2H), 8.96-9.10 (m, 2H). Mass spec m/z 304.2 [M+H]⁺.

### Step 2

### Intermediate 557: 4-(4-((1-(3-bromo-2-formylphenyl)piperidin-4-yl)oxy)piperidin-1-yl)benzamide

To a solution of 4-(4-(piperidin-4-yloxy)piperidin-1-yl)benzamide hydrochloride (Intermediate 556, 7.0 g, 23.1 mmol) in dimethyl sulfoxide (60 mL) were added N,N-diisopropylethylamine (16.1 mL, 92.3 mmol) followed by 2-bromo-6-fluorobenzaldehyde (CAS No. 360575-28-6, 4.68 g, 23.1 mmol) and the reaction mixture was heated at 100° C. for 12 h. The reaction mixture was diluted with water (100 mL), the resultant precipitate was collected by filtration and dried in vacuo. The crude material was purified by combi-flash chromatography, by eluting with 100% ethyl acetate, to afford 4-(4-((1-(3-bromo-2-formylphenyl)piperidin-4-yl)oxy)piperidin-1-yl)benzamide (Intermediate 557, 5.50 g, 49%) as a white solid. ¹H NMR (400 MHz, DMSO-d₆) δ ppm 1.45-1.53 (m, 2H), 1.60-1.68 (m, 2H), 1.82-1.95 (m, 4H), 2.90-3.02 (m, 4H), 3.12-3.16 (m, 2H), 3.55-3.68 (m, 4H), 6.92 (d, J=8.20 Hz, 2H), 6.98 (br s, 1H), 7.22 (d, J=8.23 Hz, 1H), 7.28-7.32 (m, 1H), 7.38-7.42 (m, 1H), 7.64 (br s, 1H), 7.70-7.73 (m, 2H), 10.05 (br s, 1H). Mass spec m/z 488.2 [M+H+2]⁺.

### Step 3

### Intermediate 558: 4-(4-((1-(3-bromo-2-(((2,6-dioxopiperidin-3-yl)amino)methyl)phenyl)piperidin-4-yl)oxy)piperidin-1-yl)benzamide

To a solution of 4-(4-((1-(3-bromo-2-formylphenyl)piperidin-4-yl)oxy)piperidin-1-yl)benzamide (Intermediate 557, 2.70 g, 5.60 mmol) in acetonitrile (30 mL) was added 3-aminopiperidine-2,6-dione hydrochloride (CAS No. 24666-56-6, 0.91 g, 5.60 mmol) followed by sodium acetate (0.92 g, 11.0 mmol) and 2-picoline borane complex (1.90 g, 17.0 mmol). The reaction mixture was stirred at room temperature for 16 h then concentrated in vacuo. The crude material was purified by combi-flash chromatography, by eluting with 100% ethyl acetate, to afford 4-(4-((1-(3-bromo-2-(((2,6-dioxopiperidin-3-yl)amino)methyl)phenyl)piperidin-4-yl)oxy)piperidin-1-yl)benzamide (Intermediate 558, 3.10 g, 93%) as a white solid. ¹H NMR (400 MHz, DMSO-d₆) δ ppm 1.48-1.98 (m, 10H), 2.29-2.35 (m, 2H), 2.53-2.61 (m, 2H), 2.87-3.04 (m, 4H), 3.26 (s, 2H), 3.64-3.69 (m, 4H), 3.88-3.94 (m, 1H), 6.91-6.98 (m, 2H), 7.14-7.18 (m, 1H), 7.28-7.33 (m, 2H), 7.67-7.73 (m, 2H), 10.80 (br s, 1H). Mass spec m/z 598.1 [M+H]⁺.

### Step 4

### Intermediate 559: 4-(4-((1-(2-(2,6-dioxopiperidin-3-yl)-1-oxoisoindolin-4-yl)piperidin-4-yl)oxy)piperidin-1-yl)benzamide

To a solution of 4-(4-((1-(3-bromo-2-(((2,6-dioxopiperidin-3-yl)amino)methyl)phenyl)piperidin-4-yl)oxy)piperidin-1-yl)benzamide (Intermediate 558, 1.0 g, 1.67 mmol) in 1,4-dioxane (10 mL) was added triethylamine (0.72 mL, 5.01 mmol). The reaction mixture was purged with argon for 15 min then Xantphos (0.29 g, 0.50 mmol) followed by PdCl₂(dppf) (0.25 g, 0.33 mmol) and W(CO)₆ (0.30 g, 0.83 mmol) were added. The reaction mixture was further purged with argon for 10 min and heated at 110° C. for 16 h. The reaction mixture was filtered through a celite bed and the filter pad was washed with ethyl acetate (200 mL). The filtrate was concentrated in vacuo and the crude material was purified by combi-flash chromatography, by eluting with 100% ethyl acetate, to afford 4-(4-((1-(2-(2,6-dioxopiperidin-3-yl)-1-oxoisoindolin-4-yl)piperidin-4-yl)oxy)piperidin-1-yl)benzamide (Intermediate 559, 0.55 g, 60%) as a brown solid. ¹H NMR (400 MHz, DMSO-d₆) δ ppm 1.48-1.51 (m, 2H), 1.45-1.63 (m, 2H), 1.87-1.98 (m, 6H), 2.56-2.63 (m, 1H), 2.86-2.94 (m, 4H), 3.00-3.08 (m, 2H), 3.29-3.32 (m, 1H), 3.55-3.70 (m, 4H), 4.27-4.31 (m, 1H), 4.41-4.45 (m, 1H), 5.08-5.12 (m, 1H), 6.92 (d, J=8.20 Hz, 2H), 6.98 (br s, 1H), 7.16 (d, J=8.23 Hz, 1H), 7.29 (d, J=8.13 Hz, 1H), 7.40-7.43 (m, 1H), 7.66 (br s, 1H), 7.70-7.75 (m, 2H), 10.97 (br s, 1H).

### Step 5

### Intermediate 560: tert-butyl 6-(4-(4-((1-(2-(2,6-dioxopiperidin-3-yl)-1-oxoisoindolin-4-yl)piperidin-4-yl)oxy)piperidin-1-yl)benzamido)-2-((R)-1-methylpyrrolidin-2-yl)-1H-pyrrolo[3,2-c]pyridine-1-carboxylate

To a solution of 4-(4-((1-(2-(2,6-dioxopiperidin-3-yl)-1-oxoisoindolin-4-yl)piperidin-4-yl)oxy)piperidin-1-yl)benzamide (Intermediate 559, 0.45 g, 0.84 mmol) in 1,4-dioxane (8 mL) was added tert-butyl (R)-6-bromo-2-(1-methylpyrrolidin-2-yl)-1H-pyrrolo[3,2-c]pyridine-1-carboxylate (Intermediate 4, 0.40 g, 1.05 mmol) followed by cesium carbonate (1.03 g, 3.15 mmol). The reaction mixture was purged with argon for 15 min then Xantphos (0.18 g, 0.31 mmol) and Pd₂(dba)₃ (0.14 g, 0.15 mmol) were added. The reaction mixture was further purged with argon for 10 min and heated at 100° C. for 2 h. The reaction mixture was concentrated in vacuo and the crude material purified by combi-flash chromatography, by eluting with 90% ethyl acetate in heptane, to afford tert-butyl 6-(4-(4-((1-(2-(2,6-dioxopiperidin-3-yl)-1-oxoisoindolin-4-yl)piperidin-4-yl)oxy)piperidin-1-yl)benzamido)-2-((R)-1-methylpyrrolidin-2-yl)-1H-pyrrolo[3,2-c]pyridine-1-carboxylate (Intermediate 560, 0.50 g, 56%) as white solid. Mass spec m/z 845.3 [M+H]⁺.

### Step 6

### Example 137: 4-(4-((1-(2-(2,6-dioxopiperidin-3-yl)-1-oxoisoindolin-4-yl)piperidin-4-yl)oxy)piperidin-1-yl)-N-(2-((R)-1-methylpyrrolidin-2-yl)-1H-pyrrolo[3,2-c]pyridin-6-yl)benzamide

To a cooled (0° C.) solution of tert-butyl 6-(4-(4-((1-(2-(2,6-dioxopiperidin-3-yl)-1-oxoisoindolin-4-yl)piperidin-4-yl)oxy)piperidin-1-yl)benzamido)-2-((R)-1-methylpyrrolidin-2-yl)-1H-pyrrolo[3,2-c]pyridine-1-carboxylate (Intermediate 560, 0.30 g, 0.35 mmol) in 1,4-dioxane (5 mL) was added 4M HCl in 1,4-dioxane (5 mL, 20.0 mmol) and the reaction mixture was stirred at room temperature for 4 h. The reaction mixture was then concentrated in vacuo and the crude material was purified by preparative HPLC (Method A) to afford 4-(4-((1-(2-(2,6-dioxopiperidin-3-yl)-1-oxoisoindolin-4-yl)piperidin-4-yl)oxy)piperidin-1-yl)-N-(2-((R)-1-methylpyrrolidin-2-yl)-1H-pyrrolo[3,2-c]pyridin-6-yl)benzamide (Example 137, 0.05 g, 20%) as an off-white solid. ¹H NMR (400 MHz, DMSO-d₆) δ ppm 1.50-1.62 (m, 4H), 1.80-1.99 (m, 8H), 2.11-2.15 (m, 1H), 2.16 (s, 3H), 2.21-2.26 (m, 1H), 2.41-2.45 (m, 1H), 2.53-2.61 (m, 2H), 2.87-2.92 (m, 3H), 3.05-3.15 (m, 3H), 3.26-3.30 (m, 2H), 3.68-3.70 (m, 4H), 4.27-4.32 (m, 1H), 4.41-4.46 (m, 1H), 5.08-5.13 (m, 1H), 6.37 (s, 1H), 6.98 (d, J=8.29 Hz, 2H), 7.16 (d, J=8.0 Hz, 1H), 7.28 (d, J=8.20 Hz, 1H), 7.42 (t, J=8.30 Hz, 1H), 7.94 (d, J=8.45 Hz, 2H), 8.17 (s, 1H), 8.47 (s, 1H), 10.06 (br s, 1H), 10.98 (br s, 1H), 11.31 (br s, 1H). Mass spec m/z 745.3 [M+H]⁺.

### Example 138 was Synthesised Following Scheme 134

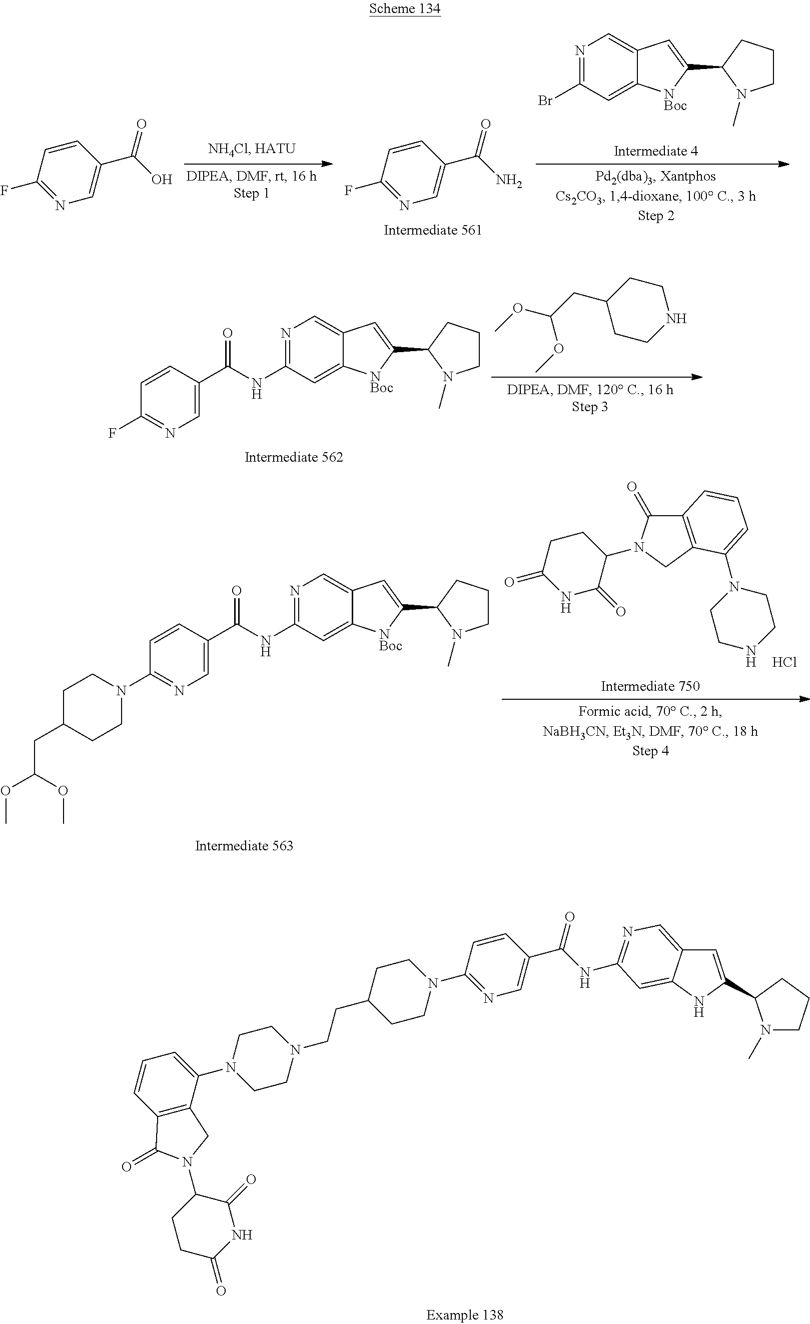

### Step 1

### Intermediate 561: 6-fluoronicotinamide

To a solution of 6-fluoronicotinic acid (CAS No: 403-45-2, 3.00 g, 21.26 mmol) in dimethylformamide (25 mL) was added HATU (12.12 g, 31.89 mmol). N,N-Diisopropylethylamine (5.29 mL, 30.3 mmol) and anhydrous ammonium chloride (5.68 g, 106.31 mmol) were then added and the reaction mixture was stirred at room temperature for 16 h. After completion, the reaction mixture was quenched with ice cold water (150 mL) and extracted with ethyl acetate (3×500 mL). The combined organic layers were dried over anhydrous Na₂SO₄, filtered, and concentrated in vacuo. The crude material was purified by combi-flash chromatography, by eluting with 5% MeOH in DCM, to afford 6-fluoronicotinamide (Intermediate 561, 1.60 g, 54%) as an off-white solid. ¹H NMR (400 MHz, DMSO-d₆) δ ppm 7.30 (dd, J=8.56, 2.69 Hz, 1H), 7.66 (br s, 1H), 8.18 (br s, 1H), 8.37-8.42 (m, 1H), 8.72 (s, 1H). Mass spec m/z 139.0 [M−H]⁻.

### Step 2

### Intermediate 562: tert-butyl (R)-6-(6-fluoronicotinamido)-2-(1-methylpyrrolidin-2-yl)-1H-pyrrolo[3,2-c]pyridine-1-carboxylate

To a suspension of 6-fluoronicotinamide (Intermediate 561, 1.45 g, 10.3 mmol) in 1,4-dioxane (25 mL) was added tert-butyl (R)-6-bromo-2-(1-methylpyrrolidin-2-yl)-1H-pyrrolo[3,2-c]pyridine-1-carboxylate (Intermediate 4, 4.33 g, 11.4 mmol) followed by cesium carbonate (5.99 g, 31.0 mmol). The reaction mixture was purged with argon for 15 min, then Xantphos (1.85 g, 3.10 mmol) and Pd₂(dba)₃ (1.32 g, 1.55 mmol) were added. The reaction mixture was further purged with argon for 10 min and heated at 100° C. for 3 h. After completion, the reaction mixture was concentrated in vacuo. The crude material was purified by combi-flash chromatography, by eluting with 10% MeOH in DCM, to afford tert-butyl (R)-6-(6-fluoronicotinamido)-2-(1-methylpyrrolidin-2-yl)-1H-pyrrolo[3,2-c]pyridine-1-carboxylate (Intermediate 562, 1.90 g, 20%) as a yellow solid. ¹H NMR (400 MHz, CDCl₃) δ ppm 1.60 (s, 9H), 1.66-1.76 (m, 2H), 1.80-1.90 (m, 2H), 2.04 (s, 3H), 2.47-2.52 (m, 1H), 3.17-3.24 (m, 1H), 3.98-4.02 (m, 1H), 6.75 (s, 1H), 7.14-7.24 (m, 1H), 8.35-8.41 (m, 1H), 8.46 (s, 1H), 8.60 (s, 1H), 8.82 (s, 1H), 8.99 (br s, 1H).

### Step 3

### Intermediate 563: tert-butyl (R)-6-(6-(4-(2,2-dimethoxyethyl)piperidin-1-yl)nicotinamido)-2-(1-methylpyrrolidin-2-yl)-1H-pyrrolo[3,2-c]pyridine-1-carboxylate

To a solution of tert-butyl (R)-6-(6-fluoronicotinamido)-2-(1-methylpyrrolidin-2-yl)-1H-pyrrolo[3,2-c]pyridine-1-carboxylate (Intermediate 562, 1.0 g, 2.28 mmol) and 4-(2,2-dimethoxyethyl)piperidine (CAS No: 15871-71-3, 0.39 g, 2.28 mmol) in dimethyl sulfoxide (20 mL) was added N,N-diisopropylethylamine (1.59 mL, 9.10 mmol) and the reaction mixture was heated at 120° C. for 16 h. After completion, the reaction mixture was concentrated in vacuo and diluted with ice cold water (100 mL). The resultant precipitate was collected by filtration, washed with n-pentane (20 mL), and dried in vacuo to afford tert-butyl (R)-6-(6-(4-(2,2-dimethoxyethyl)piperidin-1-yl)nicotinamido)-2-(1-methylpyrrolidin-2-yl)-1H-pyrrolo[3,2-c]pyridine-1-carboxylate (Intermediate 563, 1 g) as a white solid, which was used for the next step without further purification. ¹H NMR (400 MHz, DMSO-d₆) δ ppm 1.10-1.18 (m, 2H), 1.45-1.51 (m, 2H), 1.74-1.80 (m, 3H), 2.12-2.15 (m, 2H), 2.17 (s, 9H), 2.23-2.30 (m, 2H), 2.84-2.93 (m, 2H), 3.10-3.13 (m, 1H), 3.14-3.19 (m, 4H), 3.23 (s, 6H), 4.09-4.13 (m, 1H), 4.38-4.47 (m, 2H), 4.49-4.51 (m, 1H), 6.37-6.43 (m, 1H), 6.86 (d, J=9.05 Hz, 1H), 7.32-7.35 (m, 1H), 8.12-8.14 (m, 1H), 8.15-8.19 (m, 1H), 8.48-8.57 (m, 1H), 10.26 (br s, 1H). Mass spec m/z 493.2 [M−100+H]⁺.

### Step 4

### Example 138: 6-(4-(2-(4-(2-(2,6-dioxopiperidin-3-yl)-1-oxoisoindolin-4-yl)piperazin-1-yl)ethyl)piperidin-1-yl)-N-(2-((R)-1-methylpyrrolidin-2-yl)-1H-pyrrolo[3,2-c]pyridin-6-yl)nicotinamide

A mixture of tert-butyl (R)-6-(6-(4-(2,2-dimethoxyethyl)piperidin-1-yl)nicotinamido)-2-(1-methylpyrrolidin-2-yl)-1H-pyrrolo[3,2-c]pyridine-1-carboxylate (Intermediate 563 1.0 g, 2.03 mmol) and 3-(1-oxo-4-(piperazin-1-yl)isoindolin-2-yl)piperidine-2,6-dione hydrochloride (Intermediate 750, 0.90 g, 2.23 mmol) in formic acid (3 mL, 71.6 mmol) was heated at 70° C. for 2 h. The reaction mixture was concentrated in vacuo. Dimethylformamide (10 mL) and triethylamine (1.14 mL, 8.12 mmol) were then added and heating continued at 70° C. for 2 h. Sodium cyanoborohydride (0.40 g, 6.09 mmol) was then added and the reaction heated at 70° C. for 16 h. After completion, the reaction mixture was quenched with ice cold water (100 mL), the resultant precipitate was collected by filtration and dried in vacuo. The crude material was purified was purified by preparative HPLC (Method B) to afford 6-(4-(2-(4-(2-(2,6-dioxopiperidin-3-yl)-1-oxoisoindolin-4-yl)piperazin-1-yl)ethyl)piperidin-1-yl)-N-(2-((R)-1-methylpyrrolidin-2-yl)-1H-pyrrolo[3,2-c]pyridin-6-yl)nicotinamide (Example 138, 0.02 g, 1%) as an off-white solid. ¹H NMR (400 MHz, DMSO-d₆) δ ppm 1.08-1.19 (m, 2H), 1.39-1.45 (m, 2H), 1.59-1.67 (m, 1H), 1.76-1.99 (m, 8H), 2.11-2.15 (m, 1H), 2.16 (s, 3H), 2.22-2.27 (m, 1H), 2.37-2.41 (m, 4H), 2.61-2.66 (m, 2H), 2.86-2.94 (m, 3H), 3.03-3.16 (m, 6H), 4.27-4.31 (m, 1H), 4.42-4.46 (m, 2H), 5.07-5.13 (m, 1H), 6.37 (s, 1H), 6.86 (d, J=9.17 Hz, 1H), 7.16 (d, J=7.82 Hz, 1H), 7.31 (d, J=7.58 Hz, 1H), 7.41-7.45 (m, 1H), 8.11-8.14 (m, 1H), 8.16 (s, 1H), 8.18-8.20 (m, 1H), 8.47 (s, 1H), 8.78 (s, 1H), 10.23 (br s, 1H), 10.98 (br s, 1H), 11.33 (br s, 1H). Mass spec m/z 759.3 [M+H]⁺.

### Example 139 was Synthesised Following Scheme 135

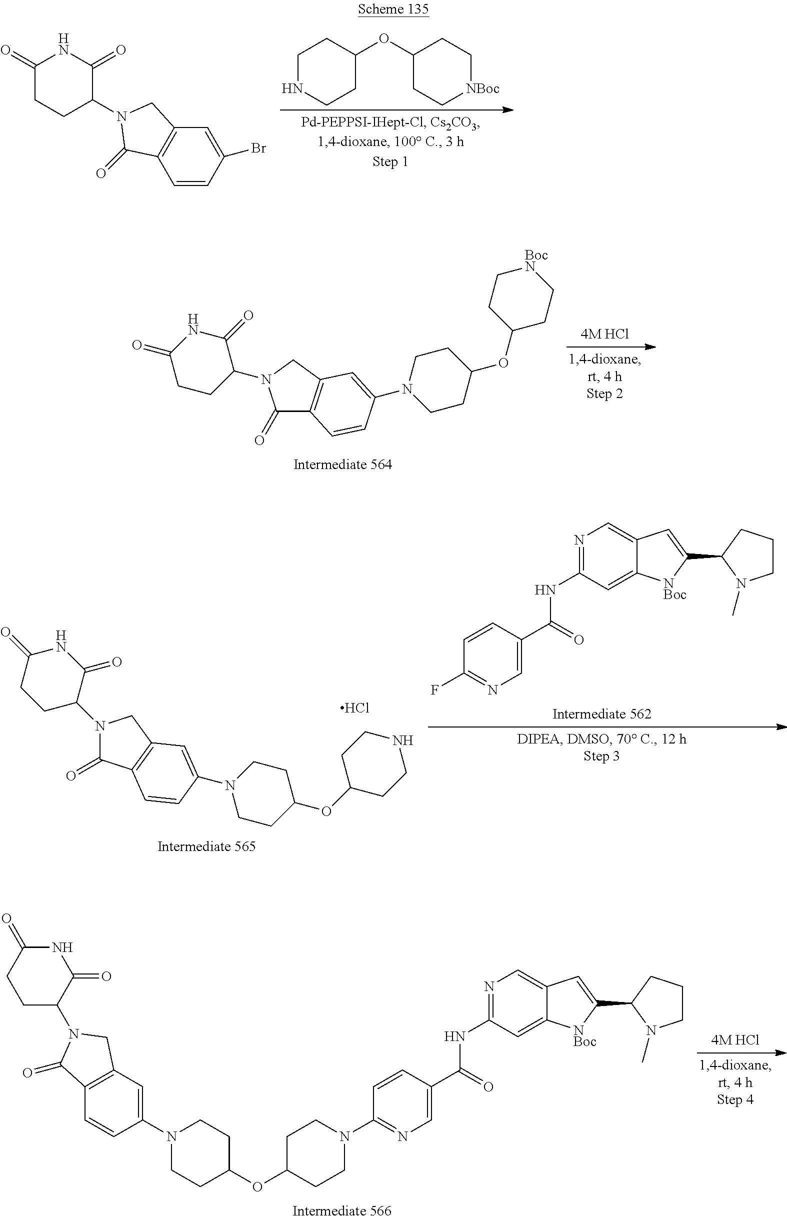

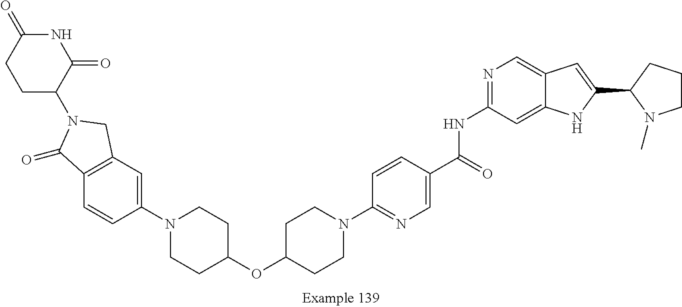 [SMILES: CN1CCC[C@@H]1c1cc2cnc(NC(=O)c3ccc(N4CCC(OC5CCN(c6ccc7c(c6)CN(C6CCC(=O)NC6=O)C7=O)CC5)CC4)nc3)cc2[nH]1]

### Step 1

### Intermediate 564: tert-butyl 4-((1-(2-(2,6-dioxopiperidin-3-yl)-1-oxoisoindolin-5-yl)piperidin-4-yl)oxy)piperidine-1-carboxylate

To a solution of 3-(5-bromo-1-oxoisoindolin-2-yl)piperidine-2,6-dione (CAS No 1010100-26-1, 2.04 g, 6.33 mmol) 1,4-dioxane (30.0 ml) was added tert-butyl 4-(piperidin-4-yloxy)piperidine-1-carboxylate (CAS No. 845305-83-1, 3.0 g, 10.5 mmol) followed by cesium carbonate (10.4 g, 31.6 mmol). The reaction mixture was purged with argon for 15 min then Pd-PEPPSI-IHept-Cl (2.16 g, 2.11 mmol) was added. The reaction mixture was purged with argon for another 10 min and stirred at 100° C. for 3 h. The reaction was then concentrated in vacuo and the crude material was purified by combi-flash chromatography, by eluting with 80-95% ethyl acetate in heptane, to afford tert-butyl 4-((1-(2-(2,6-dioxopiperidin-3-yl)-1-oxoisoindolin-5-yl)piperidin-4-yl)oxy)piperidine-1-carboxylate (Intermediate 564, 0.9 g, 16%) as an off-white solid. ¹H NMR (400 MHz, DMSO-d₆) δ ppm 1.15-1.57 (m, 15H), 1.71-1.74 (m, 2H), 1.83-1.95 (m, 3H), 2.30-2.37 (m, 1H), 2.54-2.65 (m, 2H), 2.83-3.14 (m, 4H), 3.54-3.63 (m, 4H), 4.15-4.19 (m, 1H), 4.27-4.31 (m, 1H), 5.00-5.03 (m, 1H), 7.02-7.04 (m, 2H), 7.45-7.49 (m, 1H), 10.91 (s, 1H). Mass spec m/z 527.3 [M+H]⁺.

### Step 2

### Intermediate 565: 3-(1-oxo-5-(4-(piperidin-4-yloxy)piperidin-1-yl)isoindolin-2-yl)piperidine-2,6-dione hydrochloride

To a cooled (0° C.) solution of tert-butyl 4-((1-(2-(2,6-dioxopiperidin-3-yl)-1-oxoisoindolin-5-yl)piperidin-4-yl)oxy)piperidine-1-carboxylate (Intermediate 564, 0.9 g, 1.70 mmol) in 1,4-dioxane (10 mL) was added 4M HCl in 1,4-dioxane (10 ml, 40 mmol) and the reaction mixture was stirred at room temperature for 4 h. The reaction mixture was concentrated in vacuo to afford 3-(1-oxo-5-(4-(piperidin-4-yloxy)piperidin-1-yl)isoindolin-2-yl)piperidine-2,6-dione hydrochloride (Intermediate 565, 0.7 g, 96%) as an off-white solid which was used for the next step without further purification. ¹H NMR (400 MHz, DMSO-d₆) δ ppm 1.49-1.69 (m, 5H), 1.91-1.94 (m, 4H), 2.31-2.39 (m, 1H), 2.59-2.67 (m, 2H), 2.85-2.95 (m, 3H), 3.12-3.23 (m, 4H), 3.69-3.74 (m, 4H), 4.19-4.24 (m, 1H), 4.31-4.36 (m, 1H), 5.03-5.06 (m, 1H), 7.52-7.56 (m, 1H), 8.07-8.13 (m, 1H), 8.45 (s, 1H), 10.94 (s, 1H). Mass spec m/z 427.3 [M+H]⁺.

### Step 3

### Intermediate 566: tert-butyl 6-(6-(4-((1-(2-(2,6-dioxopiperidin-3-yl)-1-oxoisoindolin-5-yl)piperidin-4-yl)oxy)piperidin-1-yl)nicotinamido)-2-((R)-1-methylpyrrolidin-2-yl)-1H-pyrrolo[3,2-c]pyridine-1-carboxylate

To a solution of 3-(1-oxo-5-(4-(piperidin-4-yloxy)piperidin-1-yl)isoindolin-2-yl)piperidine-2,6-dione hydrochloride (Intermediate 565, 0.3 g, 0.70 mmol) in dimethyl sulfoxide (10.0 ml) was added tert-butyl (R)-6-(6-fluoronicotinamido)-2-(1-methylpyrrolidin-2-yl)-1H-pyrrolo[3,2-c]pyridine-1-carboxylate (Intermediate 562, 0.3 g, 0.70 mmol) followed by N,N-diisopropylethylamine (0.49 mL, 2.81 mmol). The reaction mixture was heated at 100° C. for 12 h. The reaction mixture was quenched with water (50 mL) and extracted with ethyl acetate (2×50 mL). The combined organic layers were dried over Na₂SO₄, filtered and concentrated in vacuo. The crude material was purified by flash chromatography, by eluting with 90% ethyl acetate in heptane, to afford tert-butyl 6-(6-(4-((1-(2-(2,6-dioxopiperidin-3-yl)-1-oxoisoindolin-5-yl)piperidin-4-yl)oxy)piperidin-1-yl)nicotinamido)-2-((R)-1-methylpyrrolidin-2-yl)-1H-pyrrolo[3,2-c]pyridine-1-carboxylate (Intermediate 566, 0.37 g, 62%) as an off-white solid. Mass spec m/z 846.5 [M−100+H]⁺.

### Step 4

### Example 139: 6-(4-((1-(2-(2,6-dioxopiperidin-3-yl)-1-oxoisoindolin-5-yl)piperidin-4-yl)oxy)piperidin-1-yl)-N-(2-((R)-1-methylpyrrolidin-2-yl)-1H-pyrrolo[3,2-c]pyridin-6-yl)nicotinamide

To a cooled (0° C.) solution of tert-butyl 6-(6-(4-((1-(2-(2,6-dioxopiperidin-3-yl)-1-oxoisoindolin-5-yl)piperidin-4-yl)oxy)piperidin-1-yl)nicotinamido)-2-((R)-1-methylpyrrolidin-2-yl)-1H-pyrrolo[3,2-c]pyridine-1-carboxylate (Intermediate 566, 0.35 g, 0.41 mmol) in 1,4-dioxane (5.0 mL) was added 4M HCl in 1,4-dioxane (5.0 mL, 20 mmol) and the reaction mixture was stirred at room temperature for 4 h. The reaction mixture was concentrated in vacuo and the crude material was purified by preparative HPLC (Method A) to afford as 6-(4-((1-(2-(2,6-dioxopiperidin-3-yl)-1-oxoisoindolin-5-yl)piperidin-4-yl)oxy)piperidin-1-yl)-N-(2-((R)-1-methylpyrrolidin-2-yl)-1H-pyrrolo[3,2-c]pyridin-6-yl)nicotinamide (Example 139, 0.045 g, 15%) as an off-white solid. ¹H NMR (400 MHz, DMSO-d₆) δ ppm 1.38-1.56 (m, 4H), 1.78-1.97 (m, 9H), 2.12-2.16 (m, 4H), 2.23-2.26 (m, 1H), 2.33-2.38 (m, 1H), 2.58-2.61 (m, 1H), 2.83-2.94 (m, 1H), 3.06-3.15 (m, 3H), 3.26-3.29 (m, 2H), 3.69-3.78 (m, 4H), 4.02-4.10 (m, 2H), 4.17-4.22 (m, 1H), 4.30-4.34 (m, 1H), 5.01-5.06 (m, 1H), 6.38 (s, 1H), 6.89 (d, J=9.2 Hz, 1H), 7.05-7.07 (m, 2H), 7.50 (m, 1H), 8.13-8.16 (m, 2H), 8.48 (s, 1H), 8.78 (s, 1H), 10.26 (s, 1H), 10.94 (s, 1H), 11.34 (s, 1H). Mass spec m/z 746.5 [M+H]⁺.

### Example 140 was Synthesised Following Scheme 136

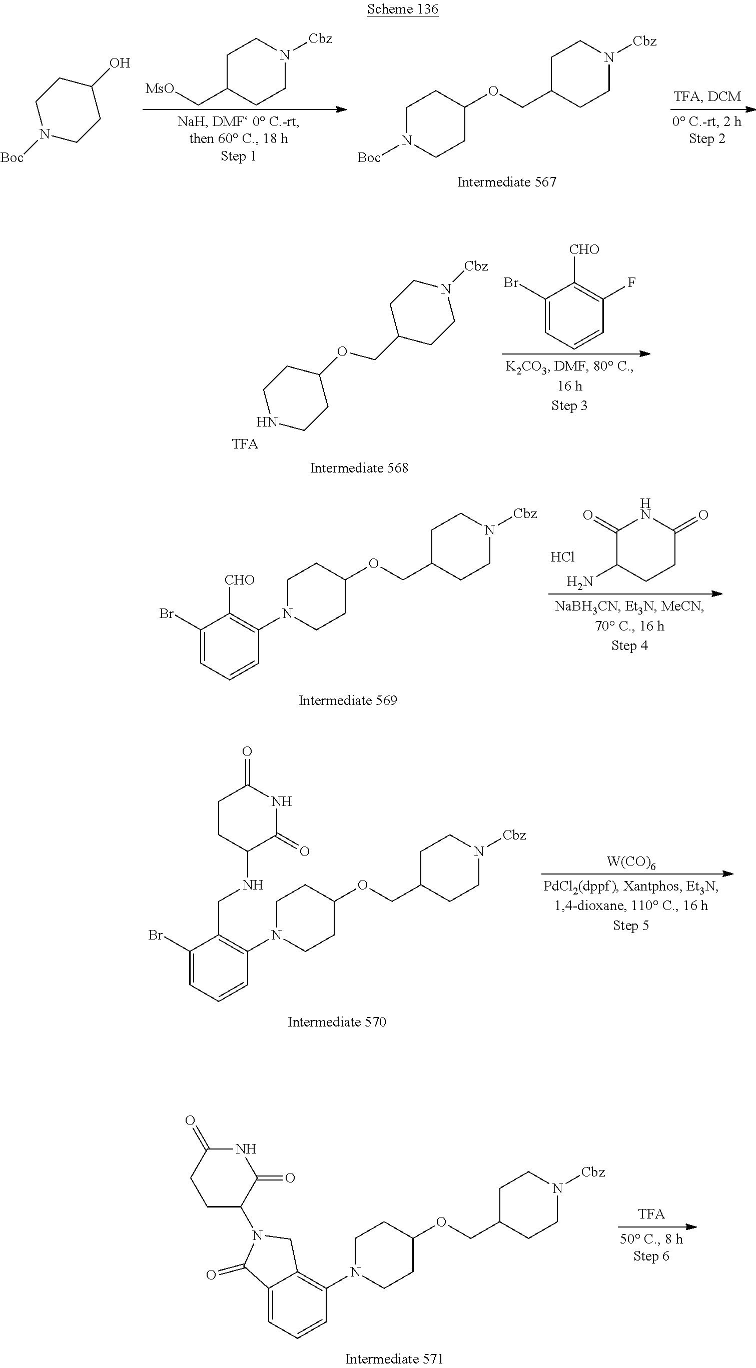

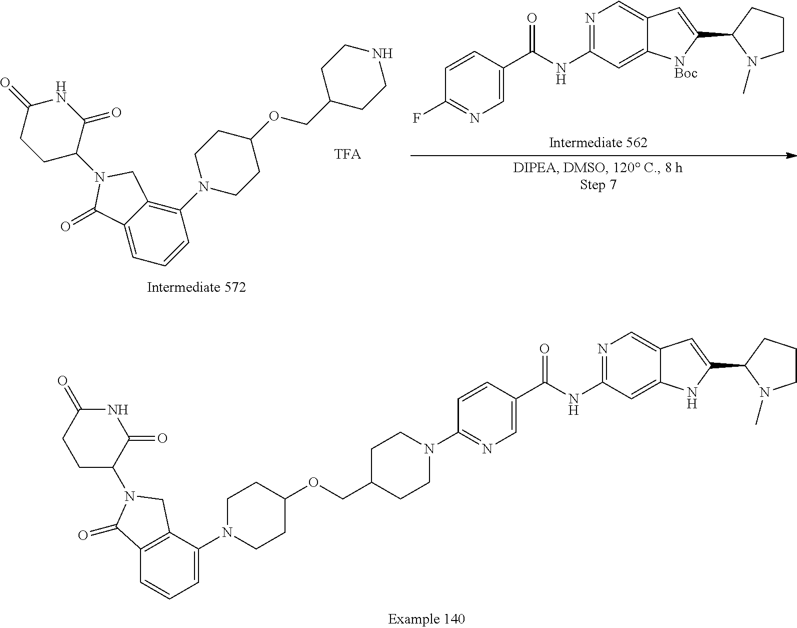

### Step 1

### Intermediate 567: benzyl 4-(((1-(tert-butoxycarbonyl)piperidin-4-yl)oxy)methyl)piperidine-1-carboxylate

To a cooled (0° C.) solution of tert-butyl 4-hydroxypiperidine-1-carboxylate (CAS No. 109384-19-2, 6.0 g, 29.81 mmol) in dimethylformamide (60 mL) was added sodium hydride (60% in mineral oil, 2.38 g, 59.62 mmol) and the reaction mixture was stirred at 0° C. for 30 min. Benzyl 4-(((methylsulfonyl)oxy)methyl)piperidine-1-carboxylate (CAS No. 159275-16-8, 10.74 g, 32.79 mmol) was then added and the reaction mixture was stirred at 70° C. for 18 h. The reaction mixture was quenched with ice cold water (150 mL) and extracted with ethyl acetate (2×200 mL). The combined organic layers were dried over Na₂SO₄, filtered and concentrated in vacuo. The crude material was purified by combi-flash chromatography, by eluting with 90% ethyl acetate in heptane, to afford benzyl 4-(((1-(tert-butoxycarbonyl)piperidin-4-yl)oxy)methyl)piperidine-1-carboxylate (Intermediate 567, 3.5 g, 27%) as an off-white solid. ¹H NMR (400 MHz, CDCl₃) δ ppm 1.10-1.21 (m, 2H), 1.42 (s, 9H), 1.58-1.82 (m, 8H), 2.70-2.76 (m, 2H), 3.06-3.12 (m, 1H), 3.26-3.42 (m, 3H), 3.60-3.73 (m, 2H), 4.12-4.24 (in, 2H), 5.08-5.12 (m, 2H), 7.27-7.34 (in, 5H). Mass spec m/z 433.4 [M+H]⁺.

### Intermediate 568: benzyl 4-((piperidin-4-yloxy)methyl)piperidine-1-carboxylate trifluoroacetate

To a cooled (0° C.) solution of benzyl 4-(((1-(tert-butoxycarbonyl)piperidin-4-yl)oxy)methyl)piperidine-1-carboxylate (Intermediate 567, 6.0 g, 13.4 mmol) in dichloromethane (36 mL) was added trifluoroacetic acid (36.0 mL) and reaction mixture was stirred at room temperature for 2 h. The reaction mixture was concentrated in vacuo to afford benzyl 4-((piperidin-4-yloxy)methyl)piperidine-1-carboxylate trifluoroacetate (Intermediate 568, 7.5 g) as a yellow gummy solid, which was used for the next step without further purification. Mass spec m/z 331.1 [M−H]⁻.

### Step 3

### Intermediate 569: benzyl 4-(((1-(3-bromo-2-formylphenyl)piperidin-4-yl)oxy)methyl)piperidine-1-carboxylate

To a solution of benzyl 4-((piperidin-4-yloxy)methyl)piperidine-1-carboxylate trifluoroacetate (Intermediate 568, 7.5 g, 16.8 mmol) in dimethylformamide (75 mL) was added potassium carbonate (8.59 g, 62 mmol) followed by 2-bromo-6-fluorobenzaldehyde (CAS No. 360575-28-6, 4.09 g, 20.16 mmol) and the reaction mixture was heated to 80° C. for 16 h. The reaction mixture was quenched with water (150 mL and extracted with ethyl acetate (3×100 mL). The combined organic layers were dried over Na₂SO₄, filtered and concentrated in vacuo. The crude material was purified by combi-flash chromatography, by eluting with 90% ethyl acetate in heptane, to afford benzyl 4-(((1-(3-bromo-2-formylphenyl)piperidin-4-yl)oxy)methyl)piperidine-1-carboxylate (Intermediate 569, 6.0 g, 57%) as a pale yellow semi-solid. ¹H NMR (400 MHz, CDCl₃) δ ppm 1.12-1.23 (m, 2H), 1.71-1.82 (m, 4H), 1.96-2.02 (m, 3H), 2.75-2.85 (m, 2H), 2.83-2.95 (m, 2H), 3.21-3.24 (m, 2H), 3.30-3.32 (m, 2H), 3.44-3.47 (m, 1H), 4.10-4.26 (m, 2H), 5.10-5.13 (m, 2H), 7.06 (s, 1H), 7.27-7.36 (m, 7H), 10.19 (s, 1H). Mass spec m/z 515.1 [M+H]⁺.

### Step 4

### Intermediate 570: benzyl 4-(((1-(3-bromo-2-(((2,6-dioxopiperidin-3-yl)amino)methyl)phenyl)piperidin-4-yl)oxy)methyl)piperidine-1-carboxylate

To a solution of 4-(((1-(3-bromo-2-formylphenyl)piperidin-4-yl)oxy)methyl)piperidine-1-carboxylate (Intermediate 569, 6.0 g, 11.6 mmol) in acetonitrile (60 mL) was added and 3-aminopiperidine-2,6-dione hydrochloride (CAS No. 24666-56-6, 2.3 g, 14 mmol) followed by triethylamine (6.49 mL, 46.56 mmol) and the reaction mixture was stirred at room temperature for 40 min. Sodium cyanoborohydride (1.46 g, 23.28 mmol) was then added and reaction mixture was heated at 70° C. for 16 h. The reaction mixture was quenched with water (100 mL) and extracted with ethyl acetate (3×100 mL). The combined organic layers were dried over Na₂SO₄ filtered, and concentrated in vacuo. The crude material was purified by combi-flash chromatography, by eluting with 80% ethyl acetate in heptane, to afford benzyl 4-(((1-(3-bromo-2-(((2,6-dioxopiperidin-3-yl)amino)methyl)phenyl)piperidin-4-yl)oxy)methyl)piperidine-1-carboxylate (Intermediate 570, 4.0 g, 55%) as a green semi-solid. ¹H NMR (400 MHz, DMSO-d₆) δ ppm 1.01-1.12 (m, 2H), 1.53-1.74 (m, 7H), 1.89-1.98 (m, 2H), 2.26-2.35 (m, 1H), 2.56-2.63 (m, 4H), 2.70-3.01 (m, 5H), 3.21-3.30 (m, 3H), 3.33-3.40 (m, 1H), 3.86-4.16 (m, 4H), 5.06 (s, 2H), 7.12-7.20 (m, 1H), 7.32-7.39 (m, 6H), 10.79 (s, 1H). Mass spec m/z 627.0 [M+H]⁺.

### Step 5

### Intermediate 571: benzyl 4-(((1-(2-(2,6-dioxopiperidin-3-yl)-1-oxoisoindolin-4-yl)piperidin-4-yl)oxy)methyl)piperidine-1-carboxylate

To a solution of benzyl 4-(((1-(3-bromo-2-(((2,6-dioxopiperidin-3-yl)amino)methyl)phenyl)piperidin-4-yl)oxy)methyl)piperidine-1-carboxylate (Intermediate 570, 1.0 g, 1.59 mmol) in 1,4-dioxane (10 mL) was added triethylamine (0.89 mL, 6.37 mmol). The reaction mixture was purged with argon gas for 15 min then Xantphos (0.18 g, 0.31 mmol), PdCl₂(dppf) (0.23 g, 0.31 mmol) followed by and tungsten hexacarbonyl (2.24 g, 6.37 mmol) were added. The reaction mixture was filtered through a celite bed and the filter pad was washed with ethyl acetate (200 mL). The filtrate was concentrated in vacuo and the crude material was purified by combi-flash chromatography, by eluting with 75% ethyl acetate in heptane, to afford benzyl 4-(((1-(2-(2,6-dioxopiperidin-3-yl)-1-oxoisoindolin-4-yl)piperidin-4-yl)oxy)methyl)piperidine-1-carboxylate (Intermediate 571, 0.68 g, 74%) as a brown solid. ¹H NMR (400 MHz, DMSO-d₆) δ ppm 0.90-1.12 (m, 2H), 1.56-1.73 (m, 5H), 1.89-2.01 (m, 3H), 2.45-2.61 (m, 3H), 2.71-2.79 (m, 5H), 3.14-3.31 (m, 4H), 3.97-4.02 (m, 2H), 4.25-4.31 (m, 1H), 4.40-4.46 (m, 1H), 5.03-5.11 (m, 3H), 7.12-7.45 (m, 8H). Mass spec m/z 575.3 [M+H]⁺.

### Step 6

### Intermediate 572: 3-(1-oxo-4-(4-(piperidin-4-ylmethoxy)piperidin-1-yl)isoindolin-2-yl)piperidine-2,6-dione trifluoroacetate

To a cooled (0° C.) solution of benzyl 4-(((1-(2-(2,6-dioxopiperidin-3-yl)-1-oxoisoindolin-4-yl)piperidin-4-yl)oxy)methyl)piperidine-1-carboxylate (Intermediate 571, 1.0 g, 1.74 mmol) was added trifluoracetic acid (50 mL). The reaction mixture was stirred at 50° C. for 8 h then concentrated in vacuo to afford as 3-(1-oxo-4-(4-(piperidin-4-ylmethoxy)piperidin-1-yl)isoindolin-2-yl)piperidine-2,6-dione trifluoroacetate (Intermediate 572, 1.7 g) as a brown solid which was used for the next step without further purification. Mass spec m/z 441.2 [M+H]⁺.

### Step 7

### Example 140: 6-(4-(((1-(2-(2,6-dioxopiperidin-3-yl)-1-oxoisoindolin-4-yl)piperidin-4-yl)oxy)methyl)piperidin-1-yl)-N-(2-((R)-1-methylpyrrolidin-2-yl)-1H-pyrrolo[3,2-c]pyridin-6-yl)nicotinamide

To a solution of 3-(1-oxo-4-(4-(piperidin-4-ylmethoxy)piperidin-1-yl)isoindolin-2-yl)piperidine-2,6-dione trifluoroacetate (Intermediate 572, 0.3 g, 0.54 mmol) in dimethyl sulfoxide (5.0 mL) was added tert-butyl (R)-6-(6-fluoronicotinamido)-2-(1-methylpyrrolidin-2-yl)-1H-pyrrolo[3,2-c]pyridine-1-carboxylate (Intermediate 562, 0.237 g, 0.7 mmol) followed by N,N-diisopropylethylamine (0.67 mL, 3.79 mmol). The reaction mixture was heated at 120° C. for 8 h. The reaction mixture was quenched with water (50 mL), the resultant precipitate was collected by filtration and dried in vacuo. The crude material was purified by preparative HPLC (Method A) to afford 6-(4-(((1-(2-(2,6-dioxopiperidin-3-yl)-1-oxoisoindolin-4-yl)piperidin-4-yl)oxy)methyl)piperidin-1-yl)-N-(2-((R)-1-methylpyrrolidin-2-yl)-1H-pyrrolo[3,2-c]pyridin-6-yl)nicotinamide (Example 140, 0.055 g, 10%) as a brown solid. ¹H NMR (400 MHz, DMSO-d₆) δ ppm 1.13-1.22 (m, 2H), 1.56-1.73 (m, 2H), 1.68-2.02 (m, 8H), 2.12-2.29 (m, 4H), 2.23-2.29 (m, 1H), 2.56-2.61 (m, 2H), 2.84-2.97 (m, 5H), 3.12-3.18 (m, 1H), 3.22-3.36 (m, 6H), 3.44-3.49 (m, 1H), 4.27-4.31 (m, 1H), 4.41-4.47 (m, 3H), 5.08-5.13 (m, 1H), 6.37 (s, 1H), 6.86 (s, 1H), 7.16 (d, J=8.40 Hz, 1H), 7.30 (d, J=6.80 Hz, 1H), 7.42 (d, J=7.60 Hz, 1H), 8.12-8.16 (m, 2H), 8.48 (m, 1H), 8.78 (m, 1H), 10.12 (s, 1H), 10.98 (s, 1H), 11.33 (s, 1H). Mass spec m/z 760.3 [M+H]⁺.

### Example 141 was Synthesised Following Scheme 137

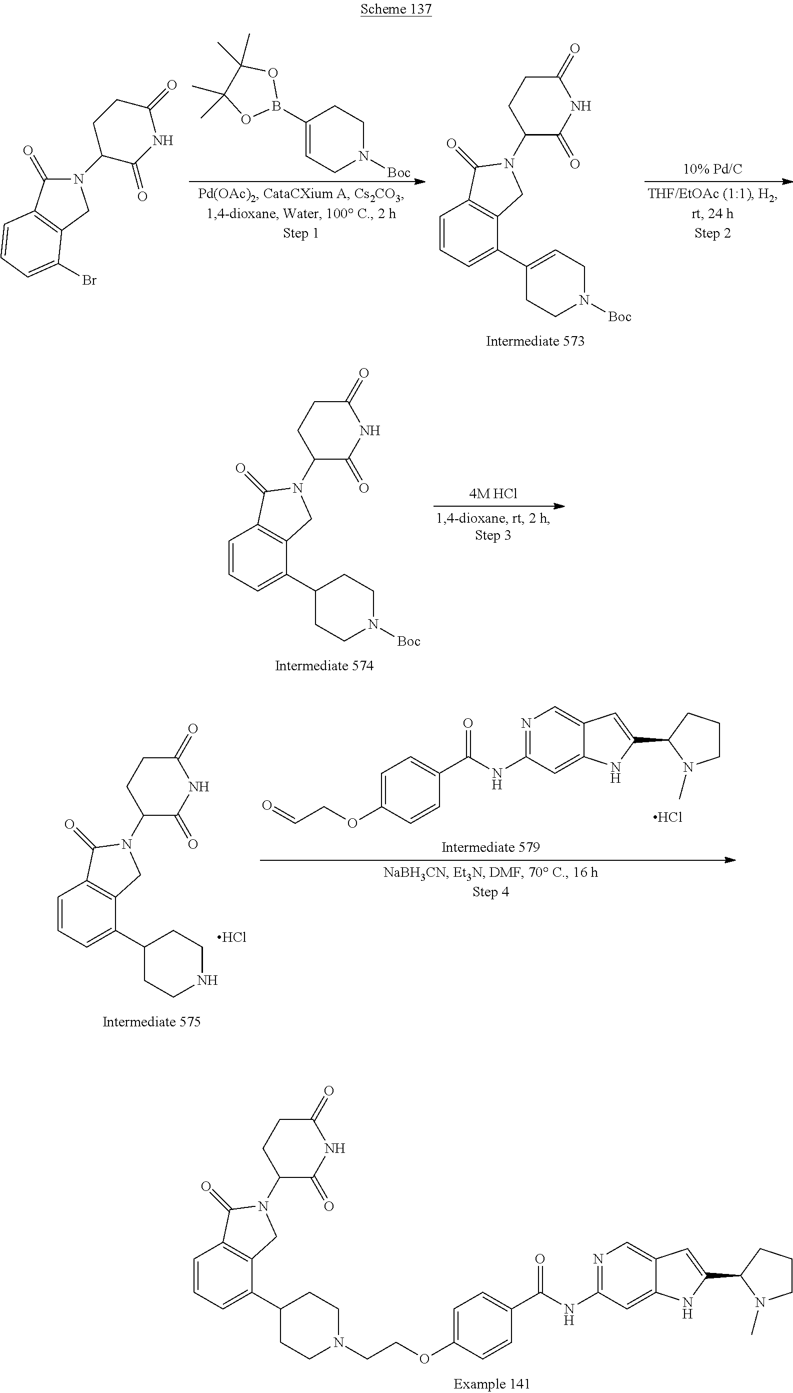

### Step 1

### Intermediate 573: tert-butyl 4-(2-(2,6-dioxopiperidin-3-yl)-1-oxoisoindolin-4-yl)-3,6-dihydropyridine-1(2H)-carboxylate

To a solution of 3-(4-bromo-1-oxoisoindolin-2-yl)piperidine-2,6-dione (CAS No. 2093387-36-9, 3.0 g, 9.28 mmol) in 1,4-dioxane (50 mL) was added tert-butyl 4-(4,4,5,5-tetramethyl-1,3,2-dioxaborolan-2-yl)-3,6-dihydropyridine-1(2H)-carboxylate (CAS No. 286961-14-6, 4.30 g, 13.9 mmol) followed by cesium carbonate (9.09 g, 27.9 mmol). The reaction mixture was purged with argon for 15 min then palladium (II) acetate (0.21 g, 0.92 mmol) and CataCXium A (0.68 g, 1.85 mmol) were added. The reaction mixture was further purged with argon for 10 min and heated at 100° C. for 2 h. The reaction mixture was concentrated in vacuo and the crude material was purified by combi-flash chromatography, by eluting with 90% ethyl acetate in heptane, to afford tert-butyl 4-(2-(2,6-dioxopiperidin-3-yl)-1-oxoisoindolin-4-yl)-3,6-dihydropyridine-1(2H)-carboxylate (Intermediate 573, 1.5 g, 38%) as an off-white solid. ¹H NMR (400 MHz, DMSO-d₆) δ ppm 1.43 (s, 9H), 1.66-1.72 (m, 1H), 1.89-2.00 (m, 2H), 2.56-2.62 (m, 2H), 2.88-2.97 (m, 1H), 3.53-3.56 (m, 2H), 4.00 (s, 2H), 4.36-4.42 (m, 1H), 4.53-4.59 (m, H), 5.11-5.16 (m, 1H), 6.02 (s, 1H), 7.50-7.58 (m, 2H), 7.63-7.65 (m, 1H), 10.99 (s, 1H). Mass spec m/z 426.1[M+H]⁺.

### Step 2

### Intermediate 574: tert-butyl 4-(2-(2,6-dioxopiperidin-3-yl)-1-oxoisoindolin-4-yl)piperidine-1-carboxylate

To a solution of tert-butyl 4-(2-(2,6-dioxopiperidin-3-yl)-1-oxoisoindolin-4-yl)-3,6-dihydropyridine-1(2H)-carboxylate (Intermediate 573, 0.45 g, 1.05 mmol) in tetrahydrofuran: ethyl acetate (1:1.10 mL) was added 10% Pd/C (0.11 g, 1.05 mmol). The mixture was stirred at room temperature for 24 h under atmospheric pressure of hydrogen. The reaction mixture was filtered through a celite bed and the filter pad was washed with methanol (100 mL). The filtrate was concentrated in vacuo to afford tert-butyl 4-(2-(2,6-dioxopiperidin-3-yl)-1-oxoisoindolin-4-yl)piperidine-1-carboxylate (Intermediate 574, 0.4 g, 88%) as an off-white solid, which was used for next step without further purification. Mass spec m/z 426.1 [M−H]⁻.

### Step 3

### Intermediate 575: 3-(1-oxo-4-(piperidin-4-yl)isoindolin-2-yl)piperidine-2,6-dione hydrochloride

To a solution of tert-butyl 4-(2-(2,6-dioxopiperidin-3-yl)-1-oxoisoindolin-4-yl)piperidine-1-carboxylate (Intermediate 574, 0.40 g, 0.93 mmol) in dichloromethane (4 mL) was added 4M hydrochloric acid in 1,4-dioxane (2.0 mL) and the reaction mixture was stirred at room temperature for 2 h. The reaction mixture was concentrated in vacuo, the residue was triturated with diethyl ether (20 mL) and dried in vacuo to afford 3-(1-oxo-4-(piperidin-4-yl)isoindolin-2-yl)piperidine-2,6-dione hydrochloride (Intermediate 575, 0.30 g, 88%) as a white solid, which was used for next step without further purification. ¹H NMR (400 MHz, DMSO-d₆) δ ppm 1.87-1.97 (m, 5H), 1.99-2.08 (m, 1H), 2.31-2.34 (m, 1H), 2.55-2.63 m, 1H), 2.93-3.00 (m, 4H), 3.32-3.41 (m, 2H), 4.35-4.41 (m, 1H), 4.53-4.59 (m, 1H), 7.44-7.50 (m, 1H), 7.54 (t, J=7.45 Hz, 1H), 7.59-7.65 (m, 1H), 9.12 (br s, 2H), 11.05 (br s, 1H). Mass spec m/z 328.3 [M+H]⁺.

### Step 4

### Example 141: 4-(2-(4-(2-(2,6-dioxopiperidin-3-yl)-1-oxoisoindolin-4-yl)piperidin-1-yl)ethoxy)-N-(2-((R)-1-methylpyrrolidin-2-yl)-1H-pyrrolo[3,2-c]pyridin-6-yl)benzamide

To a solution of 3-(1-oxo-4-(piperidin-4-yl)isoindolin-2-yl)piperidine-2,6-dione hydrochloride (Intermediate 575, 0.28 g, 0.78 mmol) in dimethylformamide (1 mL) was added triethylamine (0.33 mL, 0.05 mmol) followed by (R)—N-(2-(1-methylpyrrolidin-2-yl)-1H-pyrrolo[3,2-c]pyridin-6-yl)-4-(2-oxoethoxy)benzamide hydrochloride (Intermediate 579, 0.40 g, 0.78 mmol) and the reaction mixture was heated at 70° C. for 3 h. Sodium cyanoborohydride (0.15 g, 2.36 mmol) was then added and the reaction mixture heated at 70° C. for 16 h. The reaction mixture was diluted with ice cold water (10 mL), the resultant precipitate was collected by filtration and dried in vacuo. The crude material was purified by preparative HPLC (Method A) to afford 4-(2-(4-(2-(2,6-dioxopiperidin-3-yl)-1-oxoisoindolin-4-yl)piperidin-1-yl)ethoxy)-N-(2-((R)-1-methylpyrrolidin-2-yl)-1H-pyrrolo[3,2-c]pyridin-6-yl)benzamide (Example 141, 0.03 g, 22%) as an off-white solid. ¹H NMR (400 MHz, DMSO-d₆) δ ppm 1.83-2.01 (m, 8H), 2.11-2.16 (m, 1H), 2.17 (s, 3H), 2.17-2.24 (m, 3H), 2.39-2.46 (m, 2H), 2.57-2.64 (m, 2H), 2.76-2.80 (m, 2H), 2.87-2.97 (m, 1H), 3.06-3.17 (m, 3H), 4.19-4.20 (m, 2H), 4.33-4.38 (m, 1H), 4.51-4.55 (m, 1H), 5.11-5.16 (m, 1H), 6.38 (s, 1H), 7.05 (d, J=9.01 Hz, 2H), 7.46-7.58 (m, 3H), 8.05 (d, J=8.88 Hz, 2H), 8.17 (s, 1H), 8.49 (s, 1H), 10.28 (br s, 1H), 10.99 (br s, 1H), 11.34 (br s, 1H). Mass spec m/z 690.2 [M+H]⁺.

### Example 142 was Synthesised Following Scheme 138

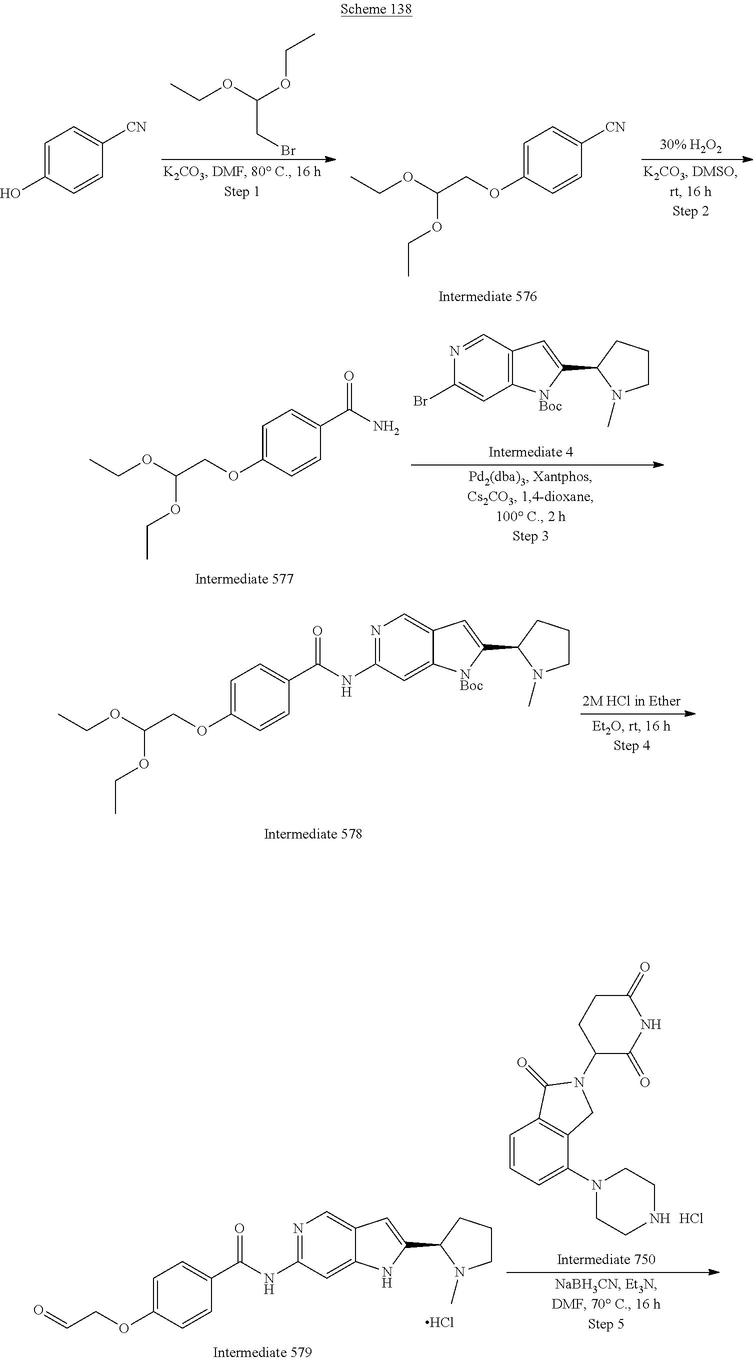

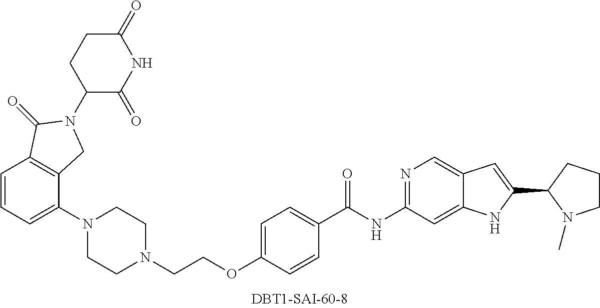 [SMILES: CN1CCC[C@@H]1c1cc2cnc(NC(=O)c3ccc(OCCN4CCN(c5cccc6c5CN(C5CCC(=O)NC5=O)C6=O)CC4)cc3)cc2[nH]1]

### Step 1

### Intermediate 576: 4-(2,2-diethoxyethoxy)benzonitrile

To a solution of 4-hydroxybenzonitrile (CAS No. 767-00-0, 5.0 g, 39.9 mmol) in dimethylformamide (50 mL) was added potassium carbonate (16.5 g, 120.0 mmol) and 2-bromo-1,1-diethoxyethane (CAS No. 2032-35-1, 8.91 g 43.9 mmol). The reaction mixture was heated at 80° C. for 16 h then diluted with ice cold water (100 mL) and extracted with ethyl acetate (3×200 mL). The combined organic layers were dried over anhydrous Na₂SO₄, filtered and concentrated in vacuo. The crude material was purified by combi-flash chromatography, by eluting with 30% ethyl acetate in heptane, to afford 4-(2,2-diethoxyethoxy)benzonitrile (Intermediate 576, 5.0 g, 53%) as a yellow solid. ¹H NMR (400 MHz, DMSO-d₆) δ ppm 1.10-1.15 (m, 6H), 3.44-3.59 (m, 2H), 3.61-3.70 (m, 2H), 4.03-4.08 (m, 2H), 4.80-4.83 (m, 1H), 7.14 (d, J=8.71 Hz, 2H), 7.76 (d, J=8.71 Hz, 2H). Mass spec m/z 236.1 [M+H]⁺.

### Step 2

### Intermediate 577: 4-(2,2-diethoxyethoxy)benzamide

To a solution of 4-(2,2-diethoxyethoxy)benzonitrile (Intermediate 576, 3.0 g, 12.5 mmol) in dimethyl sulfoxide (30 mL) was added potassium carbonate (4.31 g, 31.2 mmol) and 30% hydrogen peroxide (9.77 mL, 125.0 mmol) and the reaction mixture was stirred at room temperature for 16 h. The reaction mixture was quenched with ice cold water (100 ml) and the resultant precipitate was collected by filtration and dried in vacuo to afford 4-(2,2-diethoxyethoxy)benzamide (Intermediate 577, 2.8 g, 88%) as an off-white solid. which was used for the next step without further purification. ¹H NMR (400 MHz, DMSO-d₆) δ ppm 1.12-1.15 (m, 6H), 3.52-3.58 (m, 2H), 3.65-3.70 (m, 2H), 3.98-4.02 (m, 2H), 4.78-4.83 (m, 1H), 7.00 (d, J=8.33 Hz, 2H), 7.19 (br s, 1H), 7.83 d, J=7.89 Hz, 3H). Mass spec m/z 254.2 [M+H]⁺.

### Step 3

### Intermediate 578: tert-butyl (R)-6-(4-(2,2-diethoxyethoxy)benzamido)-2-(1-methylpyrrolidin-2-yl)-1H-pyrrolo[3,2-c]pyridine-1-carboxylate

To a solution of 4-(2,2-diethoxyethoxy)benzamide (Intermediate 577, 1.0 g, 3.94 mmol) in 1,4-dioxane (12 mL) was added tert-butyl (R)-6-bromo-2-(1-methylpyrrolidin-2-yl)-1H-pyrrolo[3,2-c]pyridine-1-carboxylate (Intermediate 4, 1.65 g, 4.34 mmol) followed by cesium carbonate (3.85 g, 11.84 mmol). The reaction mixture was purged with argon for 15 min then Pd₂(dba)₃ (0.55 g, 0.59 mmol) and Xantphos (0.35 g, 0.59 mmol) were added. The reaction mixture was further purged with argon for 10 min and heated at 100° C. for 2 h. The reaction mixture was concentrated in vacuo and the crude material was purified by combi-flash chromatography, by eluting with 80% ethyl acetate in heptane, to afford tert-butyl (R)-6-(4-(2,2-diethoxyethoxy)benzamido)-2-(1-methylpyrrolidin-2-yl)-1H-pyrrolo[3,2-c]pyridine-1-carboxylate (Intermediate 578, 1.0 g, 46%) as a yellow solid. ¹H NMR (400 MHz, DMSO-d₆) δ ppm 1.13-1.17 (m, 6H), 1.69 (s, 9H), 1.75-1.93 (m, 2H), 2.08-2.15 (m, 1H), 2.16 (m, 3H), 2.22-2.34 (m, 1H), 2.45-2.48 (m, 2H), 3.10-3.18 (m, 1H), 3.52-3.64 (m, 2H), 3.63-3.74 (m, 2H), 4.02-4.06 (m, 2H), 4.80-4.85 (m, 1H), 6.38 (s, 1H), 7.05 (d, J=8.29 Hz, 2H), 8.04 (d, J=8.29 Hz, 2H), 8.17 (s, 1H), 8.49 (s, 1H), 11.35 (br s, 1H). Mass spec m/z 553.2 [M+H]⁺.

### Step 4

### Intermediate 579: (R)—N-(2-(1-methylpyrrolidin-2-yl)-1H-pyrrolo[3,2-c]pyridin-6-yl)-4-(2-oxoethoxy)benzamide hydrochloride

To a cooled (0° C.) solution of tert-butyl (R)-6-(4-(2,2-diethoxyethoxy)benzamido)-2-(1-methylpyrrolidin-2-yl)-1H-pyrrolo[3,2-c]pyridine-1-carboxylate (Intermediate 578, 0.50 g, 0.90 mmol) in diethyl ether (8 mL) was added 2M hydrochloric acid in diethyl ether (5 mL, 10.0 mmol) and the reaction mixture was stirred at room temperature for 16 h. The reaction mixture was then concentrated in vacuo to afford (R)—N-(2-(1-methylpyrrolidin-2-yl)-1H-pyrrolo[3,2-c]pyridin-6-yl)-4-(2-oxoethoxy)benzamide hydrochloride (Intermediate 579, 0.35 g) as a brown solid, which was used for the next step without further purification. ¹H NMR (400 MHz, DMSO-d₆) δ ppm 1.13-1.23 (m, 4H), 2.16-2.20 (m, 2H), 2.81 (s, 3H), 3.57-3.79 (m, 1H), 4.71-4.78 (m, 1H), 5.03-5.08 (m, 1H), 7.10-7.22 (m, 3H), 8.17-8.23 (m, 3H), 9.06 (s, 1H), 9.70 (s, 1H), 11.42 (br s, 1H), 11.89 (br s, 1H), 13.26 (br s, 1H). Mass spec m/z 379.0 [M+H]⁺.

### Step 5

### Example 142: 4-(2-(4-(2-(2,6-dioxopiperidin-3-yl)-1-oxoisoindolin-4-yl)piperazin-1-yl)ethoxy)-N-(2-((R)-1-methylpyrrolidin-2-yl)-1H-pyrrolo[3,2-c]pyridin-6-yl)benzamide

To a solution of (R)—N-(2-(1-methylpyrrolidin-2-yl)-1H-pyrrolo[3,2-c]pyridin-6-yl)-4-(2-oxoethoxy)benzamide hydrochloride (Intermediate 579, 0.5 g, 1.20 mmol) in dimethylformamide (5.0 mL) was added 3-(1-oxo-4-(piperazin-1-yl)isoindolin-2-yl)piperidine-2,6-dione hydrochloride (Intermediate 750, 0.48 g, 1.20 mmol) followed by triethylamine (0.50 mL, 3.615 mmol) and the reaction mixture was heated at 70° C. for 2 h. Sodium cyanoborohydride (0.15 g, 2.410 mmol) was then added and the reaction mixture was stirred at 70° C. for 16 h. The reaction mixture was diluted with water (30 mL), the resultant precipitate was collected by filtration and dried in vacuo. The crude material was purified by preparative HPLC (Method A) to afford 4-(2-(4-(2-(2,6-dioxopiperidin-3-yl)-1-oxoisoindolin-4-yl)piperazin-1-yl)ethoxy)-N-(2-((R)-1-methylpyrrolidin-2-yl)-1H-pyrrolo[3,2-c]pyridin-6-yl)benzamide (Example 142, 0.01 g, 2%) as an off-white solid. ¹H NMR (400 MHz, DMSO-d₆) δ ppm 1.91-2.01 (m, 5H), 2.14-2.16 (m, 1H), 2.17 (s, 3H), 2.24-2.28 (m, 1H), 2.55-2.63 (m, 2H), 2.67-2.70 (m, 4H), 2.80-2.84 (m, 2H), 2.87-2.96 (m, 1H), 3.06-3.17 (m, 5H), 4.21-4.23 (m, 2H), 4.28-4.33 (m, 1H), 4.42-4.48 (m, 1H), 5.08-5.14 (m, 1H), 6.38 (s, 1H), 7.05 (d, J=8.88 Hz, 2H), 7.17 (d, J=7.88 Hz, 1H), 7.32 (d, J=7.50 Hz, 1H), 7.41-7.46 (m, 1H), 8.05 (d, J=8.76 Hz, 2H), 8.17 (s, 1H), 8.49 (s, 1H), 10.29 (br s, 1H), 10.98 (br s, 1H), 11.34 (br s, 1H). Mass spec m/z 691.3 [M+H]⁺.

### Example 143 was Synthesised Following Scheme 139

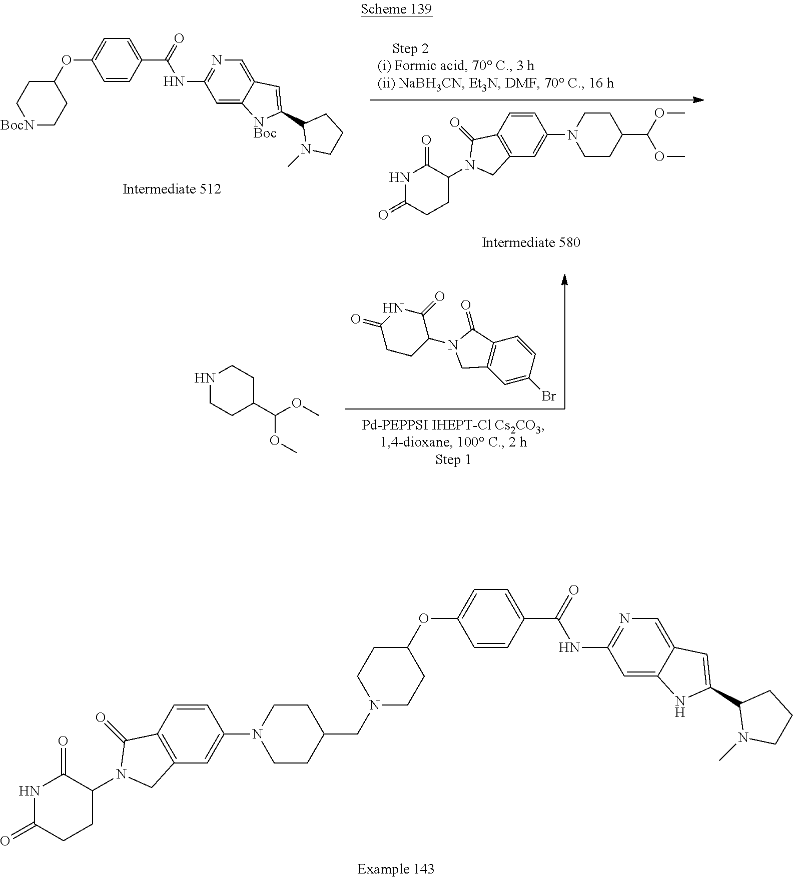

### Step 1

### Intermediate 580: 3-(5-(4-(dimethoxymethyl)piperidin-1-yl)-1-oxoisoindolin-2-yl)piperidine-2,6-dione

To a solution of 3-(5-bromo-1-oxoisoindolin-2-yl)piperidine-2,6-dione (CAS No: 1010100-26-1, 2.0 g, 6.19 mmol) in 1,4-dioxane (20 mL) was added 4-(dimethoxymethyl)piperidine (CAS No. 188646-83-5, 0.98 g, 6.19 mmol) and cesium carbonate (6.05 g, 18.57 mmol). The reaction mixture was purged with argon for 15 min then Pd-PEPPSI-IHept-Cl (1.20 g, 1.23 mmol) was added. The reaction mixture was further purged with argon for 10 min and heated at 100° C. for 2 h. The reaction mixture was diluted with water (5 mL) and extracted with ethyl acetate (15 mL). The organic layer was separated, dried over anhydrous Na₂SO₄, filtered, and concentrated in vacuo. The crude material was purified by combi-flash chromatography, by eluting with 50% ethyl acetate in heptane, to afford 3-(5-(4-(dimethoxymethyl)piperidin-1-yl)-1-oxoisoindolin-2-yl)piperidine-2,6-dione (Intermediate 580, 1.20 g, 48%) as a brown solid. Mass spec m/z 402.2 [M+H]⁺.

### Step 6

### Example 143: 4-((1-((1-(2-(2,6-dioxopiperidin-3-yl)-1-oxoisoindolin-5-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide

A solution of 3-(5-(4-(dimethoxymethyl)piperidin-1-yl)-1-oxoisoindolin-2-yl)piperidine-2,6-dione (Intermediate 580, 0.5 g, 1.24 mmol) in formic acid (5 mL) was heated at 70° C. for 3 h. The reaction mixture was concentrated in vacuo. A solution of tert-butyl (R)-6-(4-((1-(tert-butoxycarbonyl)piperidin-4-yl)oxy)benzamido)-2-(1-methylpyrrolidin-2-yl)-1H-pyrrolo[3,2-c]pyridine-1-carboxylate (Intermediate 512, 0.77 g, 1.24 mmol) in dimethylformamide (5 mL) and triethylamine (0.87 mL, 6.22 mmol) were then added and the reaction mixture was heated at 70° C. for 3 h. Sodium cyanoborohydride (0.33 g, 4.98 mmol) was then added and heating continued at 70° C. for 16 h. After completion, the reaction mixture was diluted with water (30 mL), the resultant precipitate was collected by filtration and dried in vacuo. The crude material was purified by preparative HPLC (Method B) to afford 4-((1-((1-(2-(2,6-dioxopiperidin-3-yl)-1-oxoisoindolin-5-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide (Example 143, 0.07 g, 8%) as an off-white solid. ¹H NMR (401 MHz, DMSO-d₆) δ ppm 1.13-1.25 (m, 2H), 1.66-1.70 (m, 2H), 1.71-1.96 (m, 10H), 2.10-2.15 (m, 2H), 2.16 (s, 3H), 2.18-2.29 (m, 4H), 2.34-2.42 (m, 1H), 2.57-2.63 (m, 1H), 2.68-2.74 (m, 1H), 2.78-2.96 (m, 3H), 3.11-3.18 (m, 1H), 3.28-3.31 (m, 1H), 3.85-3.92 (m, 2H), 4.16-4.21 (m, 1H), 4.28-4.33 (m, 1H), 4.47-4.51 (m, 1H), 5.02-5.06 (m, 1H), 6.38 (s, 1H), 6.99-7.08 (m, 4H), 7.50 (d, J=8.68 Hz, 1H), 8.02 (d, J=8.80 Hz, 2H), 8.17 (s, 1H), 8.48 (s, 1H), 10.29 (br s, 1H), 10.96 (br s, 1H), 11.36 (br s, 1H). Mass spec m/z 759.2 [M+H]⁺.

### Example 144 was Synthesised Following Scheme 140

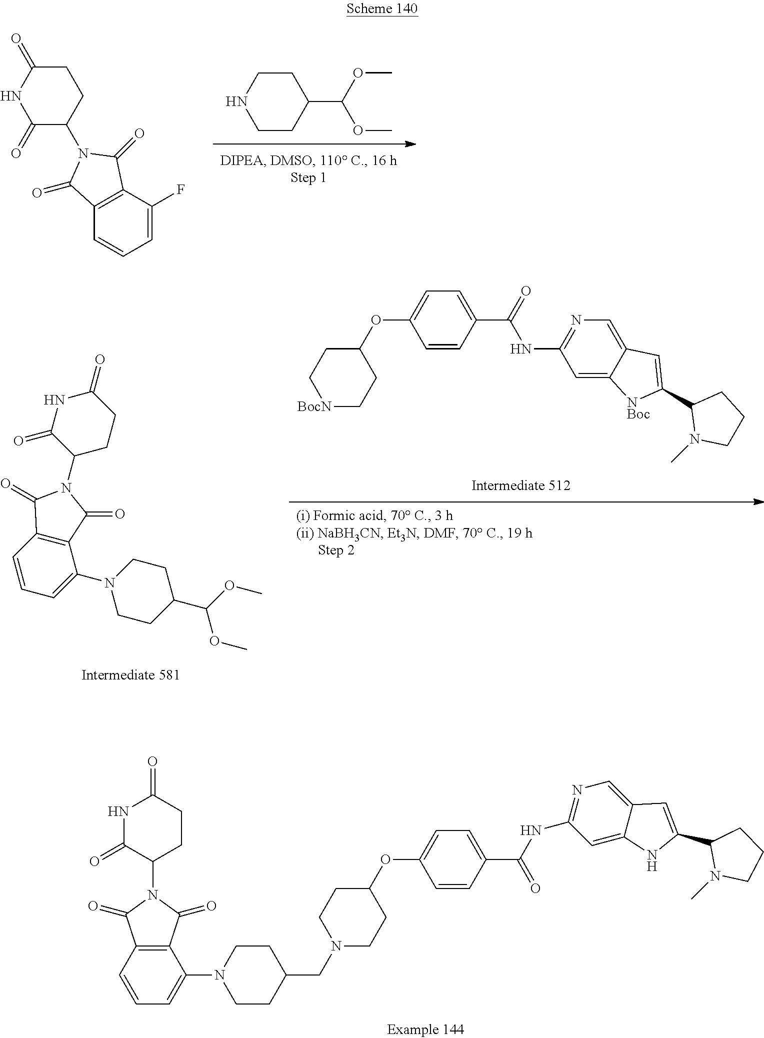

### Step 1

### Intermediate 581: 4-(4-(dimethoxymethyl)piperidin-1-yl)-2-(2,6-dioxopiperidin-3-yl)isoindoline-1,3-dione

To a solution of 4-(dimethoxymethyl)piperidine (CAS No. 188646-83-5, 1.26 g, 7.96 mmol) in dimethyl sulfoxide (20 mL) was added N,N-diisopropylethylamine (5.04 mL, 28.96 mmol) and 2-(2,6-dioxopiperidin-3-yl)-4-fluoroisoindoline-1,3-dione (CAS No. 835616-60-9, 2.0 g, 7.24 mmol). The reaction mixture was heated at 110° C. for 16 h. After completion, the reaction mixture was concentrated in vacuo. The crude material was purified by combi-flash chromatography, by eluting with 80% ethyl acetate in heptane, to afford 4-(4-(dimethoxymethyl)piperidin-1-yl)-2-(2,6-dioxopiperidin-3-yl)isoindoline-1,3-dione (Intermediate 581, 1.0 g, 33%) as an off-white solid. ¹H NMR (400 MHz, DMSO-d₆) δ ppm 1.41-1.48 (m, 2H), 1.35-1.51 (m, 2H), 1.71-1.75 (m, 2H), 2.00-2.05 (m, 1H), 2.60-2.66 (m, 1H), 2.80-2.91 (m, 3H), 3.28 (s, 6H), 3.68-3.72 (m, 2H), 4.10-4.12 (m, 1H), 5.07-5.10 (m, 1H), 7.31-7.35 (m, 2H), 7.65-7.70 (d, J=7.58 Hz, 1H), 11.08 (br s, 1H). Mass spec m/z 416.1 [M+H]⁺.

### Step 2

### Example 144: 4-((1-((1-(2-(2,6-dioxopiperidin-3-yl)-1,3-dioxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide

A solution of 4-(4-(dimethoxymethyl)piperidin-1-yl)-2-(2,6-dioxopiperidin-3-yl)isoindoline-1,3-dione (Intermediate 581, 0.50 g, 1.20 mmol) in formic acid (5 mL) was heated at 70° C. for 3 h. The reaction mixture was concentrated in vacuo. A solution of tert-butyl (R)-6-(4-((1-(tert-butoxycarbonyl)piperidin-4-yl)oxy)benzamido)-2-(1-methylpyrrolidin-2-yl)-1H-pyrrolo[3,2-c]pyridine-1-carboxylate (Intermediate 512, 0.74 g, 1.20 mmol) in dimethylformamide (5 mL) and triethylamine (0.84 mL, 6.01 mmol) were then added and the reaction mixture was heated at 70° C. for 3 h. Sodium cyanoborohydride (0.31 g, 4.85 mmol) was added and heated continued at 70° C. for 16 h. After completion, the reaction mixture was diluted with water (30 mL), the resultant precipitate was collected by filtration and dried in vacuo. The crude material was purified by preparative HPLC (Method B) to afford 4-((1-((1-(2-(2,6-dioxopiperidin-3-yl)-1,3-dioxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide (Example 144, 0.21 g, 23%) as a yellow solid. ¹H NMR (400 MHz, DMSO-d₆) δ ppm 1.26-1.36 (m, 2H), 1.61-1.74 (m, 3H), 1.77-1.91 (m, 5H), 1.94-2.06 (m, 4H), 2.16 (s, 3H), 2.19-2.28 (m, 5H), 2.54-2.58 (m, 2H), 2.66-2.75 (m, 2H), 2.82-2.93 (m, 3H), 3.10-3.16 (m, 1H), 3.34-3.36 (m, 1H), 3.65-3.78 (m, 2H), 4.47-4.56 (m, 1H), 5.06-5.09 (m, 1H), 6.38 (s, 1H), 7.03 (d, J=8.93 Hz, 2H), 7.33 (t, J=7.95 Hz, 2H), 7.64-7.71 (m, 1H), 8.02 (d, J=8.68 Hz, 2H), 8.17 (s, 1H), 8.48 (s, 1H), 10.27 (br s, 1H), 11.09 (br s, 1H), 11.34 (br s, 1H). Mass spec m/z 773.2 [M+H]⁺.

### Example 145 was Synthesised Following Scheme 141

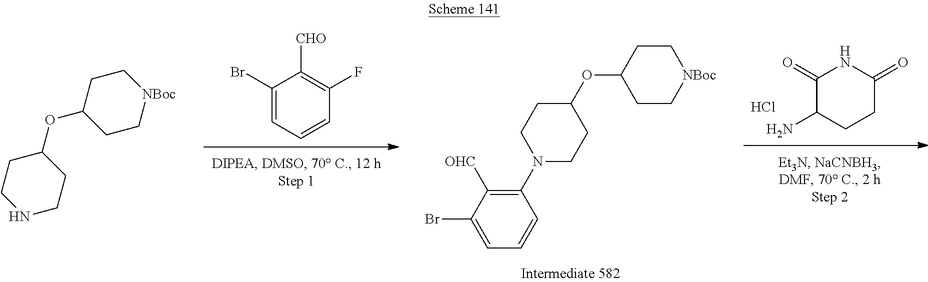 [SMILES: CC(C)(C)OC(=O)N1CCC(OC2CCNCC2)CC1 ; O=Cc1c(F)cccc1Br ; CC(C)(C)OC(=O)N1CCC(OC2CCN(c3cccc(Br)c3C=O)CC2)CC1 ; Cl ; NC1CCC(=O)NC1=O]

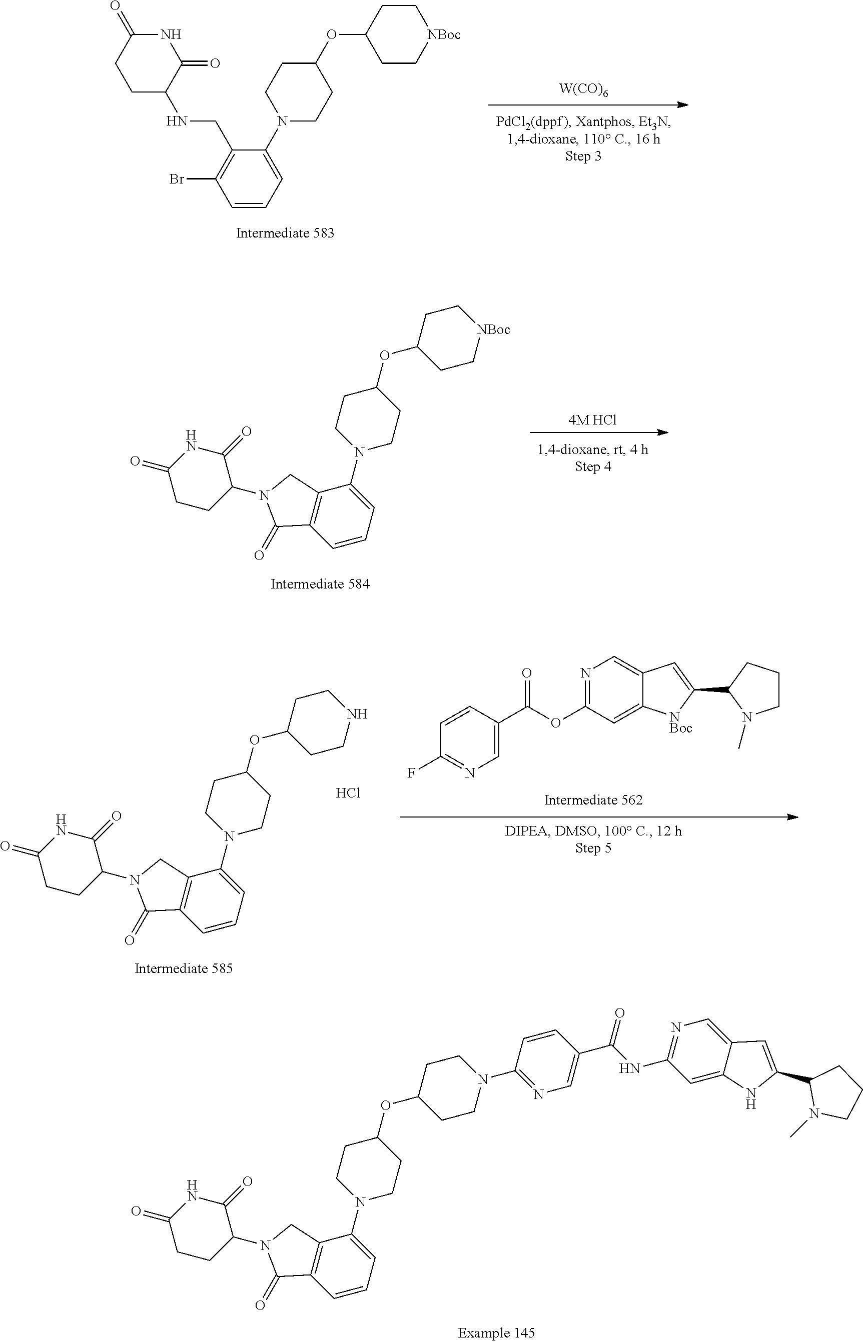

### Step 1

### Intermediate 582: tert-butyl 4-((1-(3-bromo-2-formylphenyl)piperidin-4-yl)oxy)piperidine-1-carboxylate

To a solution tert-butyl 4-(piperidin-4-yloxy)piperidine-1-carboxylate (CAS No. 845305-83-1, 3.5 g, 12.0 mmol) in dimethyl sulfoxide (30 mL) was added N,N-diisopropylethylamine (8.6 mL, 49.0 mmol) and 2-bromo-6-fluorobenzaldehyde (CAS No. 360575-28-6, 2.5 g, 12.0 mmol). The reaction mixture was purged with argon for 20 min and heated at 70° C. for 12 h. After completion, the reaction mixture was diluted with water (100 mL) and extracted with ethyl acetate (3×100 mL). The combined organic layers were dried over anhydrous Na₂SO₄, filtered and concentrated in vacuo. The crude material was purified by combi-flash chromatography, by eluting with 100% ethyl acetate in heptane, to afford tert-butyl 4-((1-(3-bromo-2-formylphenyl)piperidin-4-yl)oxy)piperidine-1-carboxylate (Intermediate 582, 3.6 g, 63%) as a colorless liquid. ¹H NMR (400 MHz, DMSO-d₆) δ ppm 1.24-1.33 (m, 2H), 1.39 (s, 9H), 1.61-1.63 (m, 2H), 1.75-1.77 (m, 2H), 1.90-1.93 (m, 2H), 2.87-2.92 (m, 2H), 2.98-3.08 (m, 2H), 3.12-3.17 (m, 2H), 3.58-3.69 (m, 4H), 7.21-7.24 (m, 1H), 7.30-7.34 (m, 1H), 7.38-7.42 (m, 1H), 10.04 (s, 1H). Mass spec m/z 467.1[M+H]⁺.

### Step 2

### Intermediate 583: tert-butyl 4-((1-(3-bromo-2-(((2,6-dioxopiperidin-3-yl)amino)methyl)phenyl)piperidin-4-yl)oxy)piperidine-1-carboxylate

To a solution of tert-butyl 4-((1-(3-bromo-2-formylphenyl)piperidin-4-yl)oxy)piperidine-1-carboxylate (Intermediate 582, 3.4 g, 7.3 mmol) and 3-aminopiperidine-2,6-dione hydrochloride (CAS No. 24666-56-6, 1.2 g, 7.3 mmol) in dimethylformamide (30 mL) was added triethylamine (4.1 mL, 29.0 mmol) followed by sodium cyanoborohydride (1.40 g, 22.0 mmol). The reaction mixture was heated at 70° C. for 2 h. After completion, the reaction mixture was diluted with water (100 mL) and extracted with ethyl acetate (3×100 mL). The combined organic layers were dried over anhydrous Na₂SO₄, filtered and concentrated in vacuo. The crude material was purified by combi-flash chromatography, by eluting with 100% ethyl acetate, to afford tert-butyl 4-((1-(3-bromo-2-(((2,6-dioxopiperidin-3-yl)amino)methyl)phenyl)piperidin-4-yl)oxy)piperidine-1-carboxylate (Intermediate 583, 2.5 g, 59%) as a white solid. ¹H NMR (400 MHz, DMSO-d₆) δ ppm 1.39 (s, 9H), 1.54-1.75 (m, 5H), 1.87-1.93 (m, 2H), 2.29-2.31 (m, 1H), 2.55-2.57 (m, 2H), 2.83-3.00 (m, 6H), 3.24-3.32 (m, 4H), 3.52-3.69 (m, 4H), 3.85-3.96 (m, 2H), 7.14-7.19 (m, 2H), 7.31-7.35 (m, 1H), 10.79 (s, 1H). Mass spec m/z 579.3 [M+H]⁺.

### Step 3

### Intermediate 584: tert-butyl 4-((1-(2-(2,6-dioxopiperidin-3-yl)-1-oxoisoindolin-4-yl)piperidin-4-yl)oxy)piperidine-1-carboxylate

To a solution of tert-butyl 4-((1-(3-bromo-2-(((2,6-dioxopiperidin-3-yl)amino)methyl)phenyl)piperidin-4-yl)oxy)piperidine-1-carboxylate (Intermediate 583, 2.5 g, 4.30 mmol) in 1,4-dioxane (20 mL) was added triethylamine (1.8 mL, 13.0 mmol). The reaction mixture was purged with argon for 15 min then tungsten hexacarbonyl (0.78 g, 2.2 mmol), PdCl₂(dppf) (0.63 g, 0.86 mmol) and Xantphos (0.77 g, 1.3 mmol) were added. The reaction mixture was further purged with argon for 10 min and heated at 110° C. for 16 h. After completion, the reaction mixture was concentrated in vacuo. The crude material was purified by combi-flash chromatography, by eluting with 100% ethyl acetate in heptane, to afford tert-butyl 4-((1-(2-(2,6-dioxopiperidin-3-yl)-1-oxoisoindolin-4-yl)piperidin-4-yl)oxy)piperidine-1-carboxylate (Intermediate 584, 1.5 g, 66%) as a brown solid. ¹H NMR (401 MHz, DMSO-d₆) δ ppm 1.28-1.35 (m, 2H), 1.39 (s, 9H), 1.55-1.64 (m, 2H), 1.72-1.80 (m, 2H), 1.88-1.97 (m, 2H), 1.97-2.03 (m, 1H), 2.42-2.48 (m, 1H), 2.56-2.62 (m, 1H), 2.81-2.94 (m, 3H), 2.95-3.08 (m, 2H), 3.24-3.31 (m, 2H), 3.58-3.69 (m, 4H), 4.26-4.31 (m, 1H), 4.31-4.45 (m, 1H), 5.09-5.14 (m, 1H), 7.17 (d, J=7.95 Hz, 1H), 7.30 (d, J=7.46 Hz, 1H), 7.40-7.46 (m, 1H), 11.01 (s, 1H). Mass spec m/z 527.4 [M+H]⁺.

### Step 4

### Intermediate 585: 3-(1-oxo-4-(4-(piperidin-4-yloxy)piperidin-1-yl)isoindolin-2-yl)piperidine-2,6-dione hydrochloride

To a cooled (0° C.) solution of tert-butyl 4-((1-(2-(2,6-dioxopiperidin-3-yl)-1-oxoisoindolin-4-yl)piperidin-4-yl)oxy)piperidine-1-carboxylate (Intermediate 584, 0.50 g, 0.94 mmol) in 1,4-dioxane (10 mL) was added 4M hydrochloric acid in 1,4-dioxane (10 mL, 40.0 mmol) and the reaction mixture was stirred at room temperature for 4 h. After completion, the reaction mixture was concentrated in vacuo to afford 3-(1-oxo-4-(4-(piperidin-4-yloxy)piperidin-1-yl)isoindolin-2-yl)piperidine-2,6-dione hydrochloride (Intermediate 585, 0.40 g, 99%) as an off-white solid, which was used for the next step without further purification. Mass spec m/z 427.3 [M+H]⁺.

### Step 5

### Example 145: 6-(4-((1-(2-(2,6-dioxopiperidin-3-yl)-1-oxoisoindolin-4-yl)piperidin-4-yl)oxy)piperidin-1-yl)-N-(2-((R)-1-methylpyrrolidin-2-yl)-1H-pyrrolo[3,2-c]pyridin-6-yl)nicotinamide

To a solution of 3-(1-oxo-4-(4-(piperidin-4-yloxy)piperidin-1-yl)isoindolin-2-yl)piperidine-2,6-dione hydrochloride (Intermediate 585, 0.4 g, 0.93 mmol) in dimethyl sulfoxide (10 mL) was added N,N-diisopropylethylamine (8.1 mL, 4.68 mmol) followed by tert-butyl (R)-6-(6-fluoronicotinamido)-2-(1-methylpyrrolidin-2-yl)-1H-pyrrolo[3,2-c]pyridine-1-carboxylate (Intermediate 562, 0.41 g, 0.93 mmol). The reaction mixture was heated at 100° C. for 12 h. After completion, the reaction mixture was diluted with water (100 mL) and the resultant precipitate was collected by filtration and dried in vacuo. The crude material was purified by preparative HPLC (Method A) to afford 6-(4-((1-(2-(2,6-dioxopiperidin-3-yl)-1-oxoisoindolin-4-yl)piperidin-4-yl)oxy)piperidin-1-yl)-N-(2-((R)-1-methylpyrrolidin-2-yl)-1H-pyrrolo[3,2-c]pyridin-6-yl)nicotinamide (Example 145, 0.05 g, 7%) as an off-white solid. ¹H NMR (400 MHz, DMSO-d₆) δ ppm 1.46-1.51 (m, 2H), 1.60-1.67 (m, 2H), 1.88-2.01 (m, 8H), 2.17 (s, 3H), 2.20-2.31 (m, 3H), 2.41-2.46 (m, 2H), 2.56-2.67 (m, 1H), 2.84-2.97 (m, 3H), 2.99-3.19 (m, 2H), 3.23-3.27 (m, 2H), 3.64-3.73 (m, 1H), 3.73-3.80 (m, 1H), 4.05-4.12 (m, 2H), 4.27-4.34 (m, 1H), 4.40-4.48 (m, 1H), 5.08-5.12 (m, 1H), 6.39 (s, 1H), 6.90 (d, J=9.13 Hz, 1H), 7.18 (d, J=7.63 Hz, 1H), 7.30 (d, J=7.25 Hz, 1H), 7.40-7.44 (m, 1H), 8.12-8.19 (m, 2H), 8.48 (s, 1H), 8.79 (s, 1H), 10.27 (br s, 1H), 10.98 (br s, 1H), 11.34 (br s, 1H). Mass spec m/z 746.4 [M+H]⁺.

### Example 146 was Synthesised Following Scheme 142

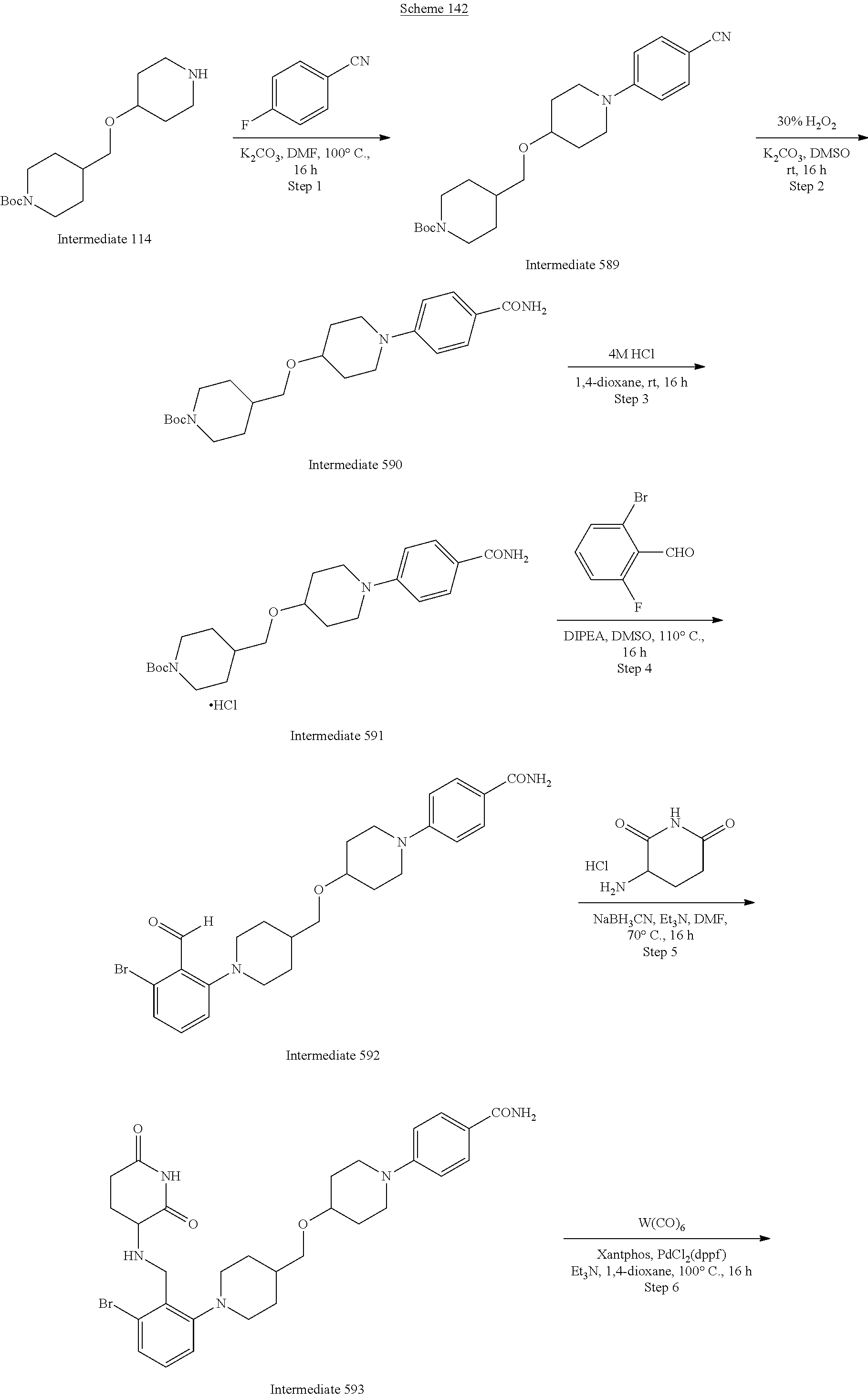

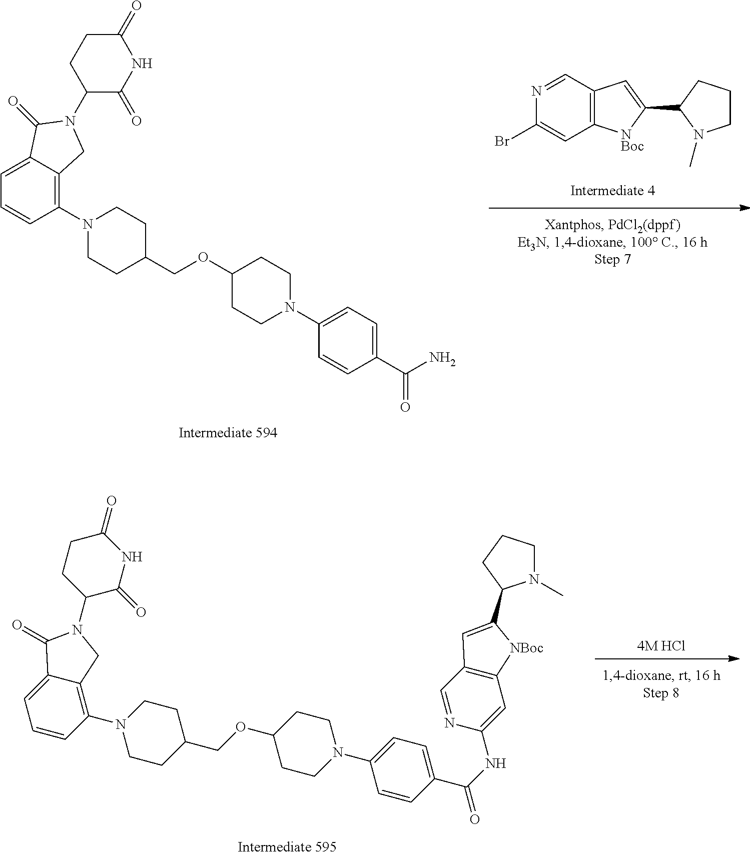

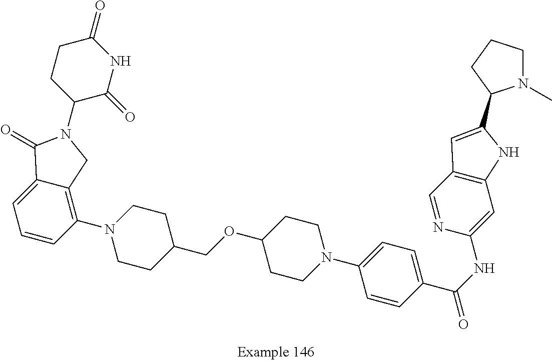 [SMILES: CN1CCC[C@@H]1c1cc2cnc(NC(=O)c3ccc(N4CCC(OCC5CCN(c6cccc7c6CN(C6CCC(=O)NC6=O)C7=O)CC5)CC4)cc3)cc2[nH]1]

### Step 1

### Intermediate 589: tert-butyl 4-(((1-(4-cyanophenyl)piperidin-4-yl)oxy)methyl)piperidine-1-carboxylate

To a solution of tert-butyl 4-((piperidin-4-yloxy)methyl)piperidine-1-carboxylate (Intermediate 114, 20.0 g, 52.95 mmol) in dimethylformamide (150 mL) was added 4-fluorobenzonitrile (CAS No. 1194-02-1, 9.61 g, 79.42 mmol) followed by potassium carbonate (22.2 g, 158.8 mmol) and the reaction mixture was heated at 100° C. for 16 h. After completion, the reaction mixture was quenched with ice cold water (500 mL) and extracted with ethyl acetate (2×500 mL). The combined organic layers were dried over anhydrous Na₂SO₄, filtered, and concentrated in vacuo. The crude material was purified by combi-flash chromatography, by eluting with 30% ethyl acetate in heptane, to afford tert-butyl 4-(((1-(4-cyanophenyl)piperidin-4-yl)oxy)methyl)piperidine-1-carboxylate (Intermediate 589, 18.1 g, 71%) as a yellow solid. ¹H NMR (400 MHz, DMSO-d₆) δ ppm 1.00-1.07 (m, 2H), 1.38 (s, 9H), 1.42-1.47 (m, 2H), 1.60-1.65 (m, 3H), 1.80-1.90 (m, 2H), 2.62-2.75 (m, 2H), 3.10-3.19 (m, 2H), 3.26-3.30 (m, 2H), 3.46-3.54 (m, 1H), 3.59-3.68 (m, 2H), 3.90-3.96 (m, 2H), 7.01 (d, J=8.31 Hz, 2H), 7.54 (d, J=8.69 Hz, 2H). Mass spec m/z 400.1 [M+H]⁺.

### Step 2

### Intermediate 590: tert-butyl 4-(((1-(4-carbamoylphenyl)piperidin-4-yl)oxy)methyl)piperidine-1-carboxylate

To a solution of tert-butyl 4-(((1-(4-cyanophenyl)piperidin-4-yl)oxy)methyl)piperidine-1-carboxylate (Intermediate 589, 18.0 g, 45.06 mmol) in dimethyl sulfoxide (170 mL) was added potassium carbonate (15.54 g, 112.6 mmol) followed by 30% hydrogen peroxide (35.2 mL, 450.6 mmol). The reaction mixture was stirred at room temperature for 16 h. After completion, the reaction mixture was diluted with water (50 mL), the resultant precipitate was collected by filtration and dried in vacuo to afford tert-butyl 4-(((1-(4-carbamoylphenyl)piperidin-4-yl)oxy)methyl)piperidine-1-carboxylate (Intermediate 590, 14.0 g, 74%) as a white solid, which was used for the next step without further purification. ¹H NMR (400 MHz, DMSO-d₆) δ ppm 0.96-1.08 (m, 2H), 1.37 (s, 9H), 1.38-1.41 (m, 1H), 1.45-1.54 (m, 2H), 1.61-1.70 (m, 3H), 1.82-1.91 (m, 2H), 2.64-2.74 (m, 1H), 2.98-3.08 (m, 2H), 3.26-3.30 (m, 2H), 3.41-3.49 (m, 1H), 3.52-3.61 (m, 2H), 3.85-3.97 (m, 2H), 6.92 (d, J=8.69 Hz, 2H), 6.98 (br s, 1H), 7.67 (br s, 1H), 7.72 (d, J=8.69 Hz, 2H). Mass spec m/z 418.2 [M+H]⁺.

### Step 3

### Intermediate 591: 4-(4-(piperidin-4-ylmethoxy)piperidin-1-yl)benzamide hydrochloride

To a cooled (0° C.) solution of tert-butyl 4-(((1-(4-carbamoylphenyl)piperidin-4-yl)oxy)methyl)piperidine-1-carboxylate (Intermediate 590, 7.0 g, 16.77 mmol) in 1,4-dioxane (70 mL) was added 4M hydrochloric acid in 1,4-dioxane (70 mL) and the reaction mixture was stirred at room temperature for 16 h. After completion, the reaction mixture was concentrated in vacuo to afford 4-(4-(piperidin-4-ylmethoxy)piperidin-1-yl)benzamide hydrochloride (Intermediate 591, 7.1 g) as an off-white semi-solid, which was used for the next step without further purification. Mass spec m/z 318.1 [M+H]⁺.

### Step 4

### Intermediate 592: 4-(4-((1-(3-bromo-2-formylphenyl)piperidin-4-yl)methoxy)piperidin-1-yl)benzamide

To a solution of 4-(4-(piperidin-4-ylmethoxy)piperidin-1-yl)benzamide hydrochloride (Intermediate 591, 1.0 g, 3.15 mmol) in dimethyl sulfoxide (5 mL) was added 2-bromo-6-fluorobenzaldehyde (CAS No. 360575-28-6, 0.73 g, 3.46 mmol) and N,N-diisopropylethylamine (1.65 mL, 9.45 mmol). The reaction mixture was heated at 110° C. for 16 h. After completion, the reaction mixture was diluted with water (50 mL) and extracted with ethyl acetate (2×80 mL). The combined organic layers were dried over anhydrous Na₂SO₄, filtered and concentrated in vacuo. The crude material was purified by combi-flash chromatography, by eluting with 50% ethyl acetate in heptane, to afford 4-(4-((1-(3-bromo-2-formylphenyl)piperidin-4-yl)methoxy)piperidin-1-yl)benzamide (Intermediate 592, 0.8 g, 43%) as a yellow sticky liquid. ¹H NMR (400 MHz, DMSO-d₆) δ ppm 1.36-1.40 (m, 2H), 1.48-1.56 (m, 2H), 1.63-1.67 (m, 1H), 1.74-1.81 (m, 2H), 1.86-1.94 (m, 2H), 2.82-2.88 (m, 2H), 3.02-3.07 (m, 2H), 3.16-3.19 (m, 2H), 3.37-3.43 (m, 2H), 3.45-3.54 (m, 1H), 3.56-3.64 (m, 2H), 6.93 (d, J=8.22 Hz, 2H), 6.98 (br s, 1H), 7.22 (d, J=7.83 Hz, 1H), 7.31 (d, J=7.43 Hz, 1H), 7.36-7.47 (m, 1H), 7.67 (br s, 1H), 7.73 (d, J=8.61 Hz, 2H), 10.03 (br s, 1H). Mass spec m/z 500.0 [M+H]⁺.

### Step 5

### Intermediate 593: 4-(4-((1-(3-bromo-2-(((2,6-dioxopiperidin-3-yl)amino)methyl)phenyl)piperidin-4-yl)methoxy)piperidin-1-yl)benzamide

To a solution of 4-(4-((1-(3-bromo-2-formylphenyl)piperidin-4-yl)methoxy)piperidin-1-yl)benzamide (Intermediate 592, 6.5 g, 13.0 mmol) in dimethylformamide (50 mL) was added 3-aminopiperidine-2,6-dione hydrochloride (CAS No. 24666-56-6, 2.4 g, 14.0 mmol) and triethylamine (7.3 mL, 52.0 mmol). A solution of sodium cyanoborohydride (2.6 g, 39.0 mmol) in dimethylformamide (50 mL) was then added and the reaction mixture heated at 70° C. for 16 h. After completion, reaction mixture was concentrated in vacuo, diluted with ice cold water (500 mL), the resultant precipitate was collected by filtration and dried in vacuo to afford 4-(4-((1-(3-bromo-2-(((2,6-dioxopiperidin-3-yl)amino)methyl)phenyl)piperidin-4-yl)methoxy)piperidin-1-yl)benzamide (Intermediate 593, 5.7 g) as an off-white solid, which was for the next step without further purification. ¹H NMR (400 MHz, DMSO-d₆), δ ppm 1.09-1.13 (m, 2H), 1.30-1.88 (m, 10H), 2.28-2.32 (m, 1H), 2.51-2.58 (m, 2H), 2.73-2.87 (m, 1H), 2.92-3.10 (m, 4H), 3.20-3.30 (m, 2H), 3.34-3.39 (m, 2H), 3.45-3.60 (m, 3H), 3.89-3.92 (m, 1H), 6.93 (d, J=8.22 Hz, 2H), 6.98 (br s, 1H), 7.15-7.20 (m, 2H), 7.33 (d, J=7.04 Hz, 1H), 7.67 (br s, 1H), 7.72 (d, J=8.22 Hz, 2H), 10.78 (br s, 1H). Mass spec m/z 614.1 [M+H]⁺.

### Step 6

### Intermediate 594: 4-(4-((1-(2-(2,6-dioxopiperidin-3-yl)-1-oxoisoindolin-4-yl)piperidin-4-yl)methoxy)piperidin-1-yl)benzamide

To a solution of 4-(4-((1-(3-bromo-2-(((2,6-dioxopiperidin-3-yl)amino)methyl)phenyl)piperidin-4-yl)methoxy)piperidin-1-yl)benzamide (Intermediate 593, 5.6 g, 9.1 mmol) in 1,4-dioxane (35 mL) was added triethylamine (3.8 mL, 27.0 mmol). The reaction mixture was purged with argon for 15 min then tungsten hexacarbonyl (1.7 g, 4.6 mmol), PdCl₂(dppf) (1.4 g, 1.8 mmol) and Xantphos (1.1 g, 1.8 mmol) were added. The reaction mixture was further purged with argon for 10 min and heated at 100° C. for 16 h. After completion, the reaction mixture was concentrated in vacuo. The crude material was purified by combi-flash chromatography, by eluting with 15% MeOH in DCM, to afford 4-(4-((1-(2-(2,6-dioxopiperidin-3-yl)-1-oxoisoindolin-4-yl)piperidin-4-yl)methoxy)piperidin-1-yl)benzamide (Intermediate 594, 3.5 g, 68%) as a brown solid. Mass spec m/z 560.3 [M+H]⁺.

### Step 7

### Intermediate 595: tert-butyl 6-(4-(4-((1-(2-(2,6-dioxopiperidin-3-yl)-1-oxoisoindolin-4-yl)piperidin-4-yl)methoxy)piperidin-1-yl)benzamido)-2-((R)-1-methylpyrrolidin-2-yl)-1H-pyrrolo[3,2-c]pyridine-1-carboxylate

To a solution of 4-(4-((1-(2-(2,6-dioxopiperidin-3-yl)-1-oxoisoindolin-4-yl)piperidin-4-yl)methoxy)piperidin-1-yl)benzamide (Intermediate 594, 1.2 g, 2.14 mmol) in 1,4-dioxane (3 mL) was added tert-butyl (R)-6-bromo-2-(1-methylpyrrolidin-2-yl)-1H-pyrrolo[3,2-c]pyridine-1-carboxylate (Intermediate 4, 0.89 g, 2.35 mmol) and cesium carbonate (2.09 g, 6.43 mmol). The reaction mixture was purged with argon gas for 15 min then Xantphos (0.38 g, 0.64 mmol) and Pd₂(dba)₃ (0.30 g, 0.32 mmol) were added. The reaction mixture was further purged with argon gas for 10 min and heated at 100° C. for 2 h. After completion, reaction mixture was filtered through filter paper and the filtrate concentrated in vacuo. The crude material was purified by combi-flash chromatography, by eluting with 7% MeOH in DCM, to afford tert-butyl 6-(4-(4-((1-(2-(2,6-dioxopiperidin-3-yl)-1-oxoisoindolin-4-yl)piperidin-4-yl)methoxy)piperidin-1-yl)benzamido)-2-((R)-1-methylpyrrolidin-2-yl)-1H-pyrrolo[3,2-c]pyridine-1-carboxylate (Intermediate 595, 0.53 g, 29%) as an off-white solid. ¹H NMR (400 MHz, DMSO-d₆) δ ppm 1.29-1.38 (m, 4H), 1.52-1.60 (m, 4H), 1.69 (s, 9H), 1.72-1.84 (m, 7H), 1.89-1.95 (s, 3H), 1.98 (s, 3H), 2.65-2.77 (m, 2H), 2.87-2.95 (m, 2H), 3.08-3.13 (m, 2H), 3.34-3.40 (m, 4H), 3.50-3.57 (m, 1H), 3.60-3.66 (m, 2H), 4.26-4.30 (m, 1H), 4.40-4.45 (m, 1H), 5.09-5.15 (m, 1H), 6.37 (s, 1H), 6.95-7.00 (m, 2H), 7.13-7.18 (m, 1H), 7.25-7.32 (m, 1H), 7.39-7.46 (m, 1H), 7.91-7.98 (m, 2H), 8.56 (s, 1H), 8.91 (s, 1H), 10.32 (br s, 1H), 10.98 (br s, 1H). Mass spec m/z 859.3 [M+H]⁺.

### Step 8

### Example 146: 4-(4-((1-(2-(2,6-dioxopiperidin-3-yl)-1-oxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)benzamide

To a cooled (0° C.) solution of tert-butyl 6-(4-(4-((1-(2-(2,6-dioxopiperidin-3-yl)-1-oxoisoindolin-4-yl)piperidin-4-yl)methoxy)piperidin-1-yl)benzamido)-2-((R)-1-methylpyrrolidin-2-yl)-1H-pyrrolo[3,2-c]pyridine-1-carboxylate (Intermediate 595, 0.52 g, 0.60 mmol) in 1,4-dioxane (2.0 mL) was added 4M hydrochloric acid in 1,4-dioxane (2 mL) and the reaction mixture was stirred at room temperature for 16 h. After completion, the reaction mixture was concentrated in vacuo, the residue was triturated with diethyl ether and dried in vacuo. The crude material was purified by preparative HPLC (Method A) to afford 4-(4-((1-(2-(2,6-dioxopiperidin-3-yl)-1-oxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)benzamide (Example 146, 0.108 g, 24%) as an off-white solid. ¹H NMR (400 MHz, DMSO-d₆) δ ppm 1.23-1.37 (m, 2H), 1.52-1.58 (m, 2H), 1.64-1.75 (m, 1H), 1.76-1.99 (m, 8H), 2.17 (s, 3H), 2.20-2.31 (m, 2H), 2.52-2.63 (m, 2H), 2.65-2.77 (m, 2H), 2.87-2.96 (m, 1H), 3.08-3.13 (m, 3H), 3.24-3.30 (m, 1H), 3.32-3.41 (m, 4H), 3.50-3.57 (m, 1H), 3.60-3.66 (m, 2H), 4.26-4.30 (m, 1H), 4.40-4.45 (m, 1H), 5.08-5.13 (m, 1H), 6.38 (s, 1H), 6.98 (d, J=8.80 Hz, 2H), 7.16 (d, J=7.95 Hz, 1H), 7.30 (d, J=7.46 Hz, 1H), 7.39-7.46 (m, 1H), 7.95 (d, J=8.80 Hz, 2H), 8.17 (s, 1H), 8.48 (s, 1H), 10.07 (s, 1H), 10.97 (s, 1H), 11.33 (br s, 1H). Mass spec m/z 759.4 [M+H]⁺.

### Example 147 was Synthesised Following Scheme 143

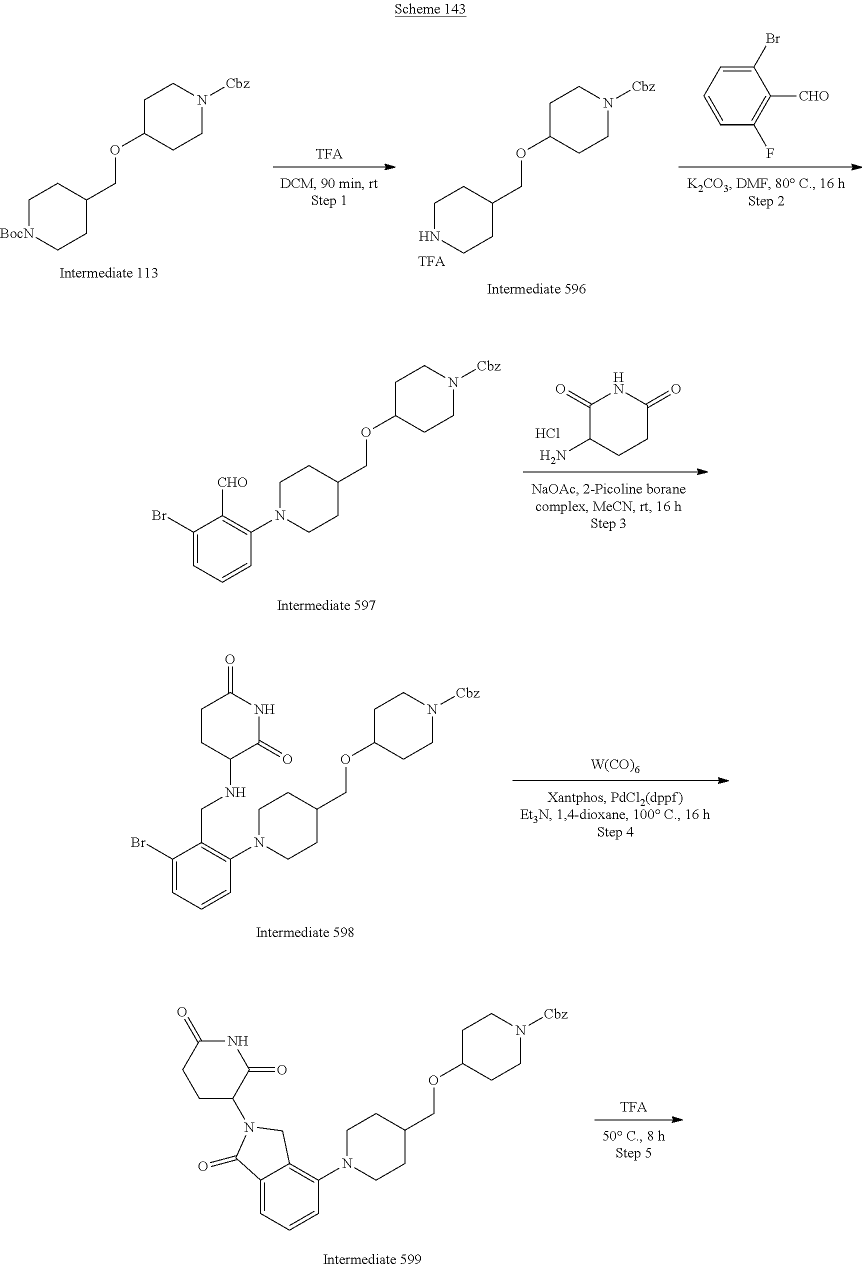

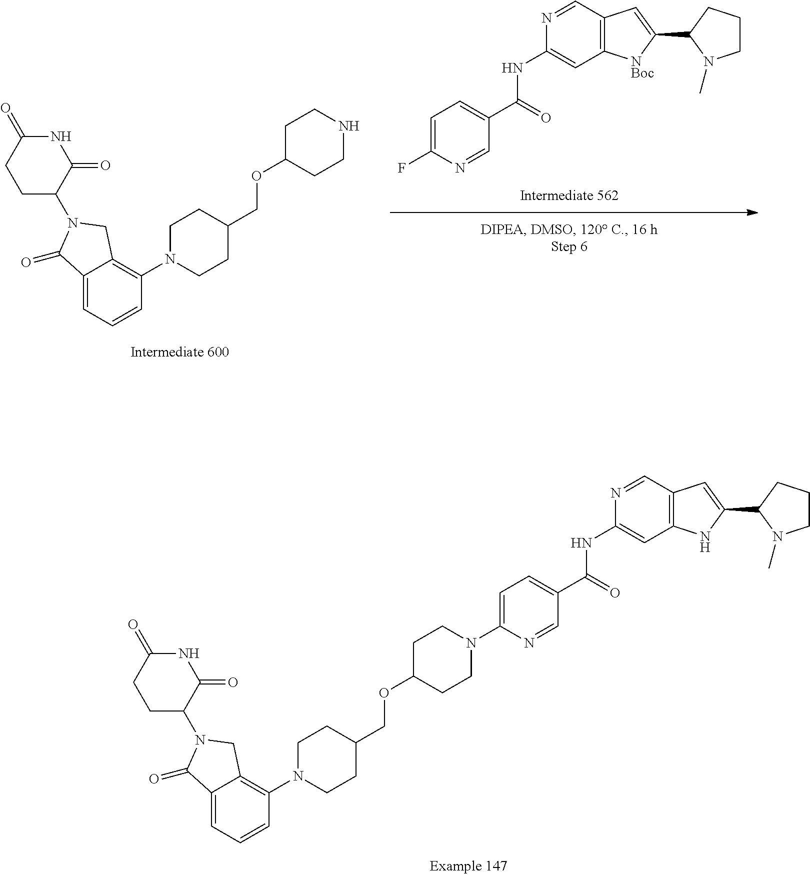

### Step 1

### Intermediate 596: benzyl 4-(piperidin-4-ylmethoxy)piperidine-1-carboxylate trifluoroacetate

To a cooled (0° C.) solution of benzyl 4-((1-(tert-butoxycarbonyl)piperidin-4-yl)methoxy)piperidine-1-carboxylate (Intermediate 113, 11.0 g, 26.40 mmol) in dichloromethane (55 mL) was added trifluoroacetic acid (55 mL) and the reaction mixture was stirred at room temperature for 90 min. After completion, the reaction mixture was concentrated in vacuo to afford benzyl 4-(piperidin-4-ylmethoxy)piperidine-1-carboxylate trifluoroacetate (Intermediate 596, 10.2 g) as a colourless oil, which was used for the next step without further purification. ¹H NMR (400 MHz, DMSO-d₆) δ ppm 1.30-1.38 (m, 5H), 1.72-1.77 (m, 6H), 2.60-2.69 (m, 2H), 2.80-2.89 (m, 3H), 3.24-3.4 (m, 6H), 5.07 (s, 2H), 7.30-7.39 (m, 5H).

### Step 2

### Intermediate 597: benzyl 4-((1-(3-bromo-2-formylphenyl)piperidin-4-yl)methoxy)piperidine-1-carboxylate

To a solution of benzyl 4-(piperidin-4-ylmethoxy)piperidine-1-carboxylate trifluoroacetate (Intermediate 596, 10.0 g, 23.01 mmol) in dimethylformamide (100 mL) was added 2-bromo-6-fluorobenzaldehyde (CAS No. 360575-28-6, 4.67 g, 23.01 mmol) and potassium carbonate (11.13 g, 80.53 mmol). The reaction mixture was heated at 80° C. for 16 h. After completion, the reaction mixture was quenched with ice cold water (200 mL) and the resultant precipitate was collected by filtration and dried in vacuo. The crude material was purified by combi-flash chromatography, by eluting with 15% ethyl acetate in heptane, to afford benzyl 4-((1-(3-bromo-2-formylphenyl)piperidin-4-yl)methoxy)piperidine-1-carboxylate (Intermediate 597, 9.10 g, 77%) as a yellow solid. ¹H NMR (400 MHz, DMSO-d₆) δ ppm 1.30-1.46 (m, 4H), 1.66-1.70 (m, 1H), 1.74-1.83 (m, 4H), 2.81-2.89 (m, 2H), 3.15-3.25 (m, 4H), 3.34-3.36 (m, 1H), 3.42-3.52 (m, 2H), 3.60-3.69 (m, 2H), 5.06 (s, 2H), 7.21-7.27 (m, 2H), 7.29-7.48 (m, 6H), 10.02 (br s, 1H). Mass spec m/z 517.1 [M+H]⁺.

### Step 3

### Intermediate 598: benzyl 4-((1-(3-bromo-2-(((2,6-dioxopiperidin-3-yl)amino)methyl)phenyl)piperidin-4-yl)methoxy)piperidine-1-carboxylate

To a solution of benzyl 4-((1-(3-bromo-2-formylphenyl)piperidin-4-yl)methoxy)piperidine-1-carboxylate (Intermediate 597, 1.0 g, 1.94 mmol) and 3-aminopiperidine-2,6-dione hydrochloride (CAS No. 1909304-98-8, 0.38 g, 2.32 mmol) in acetonitrile (30 mL) was added sodium acetate (0.24 g, 2.91 mmol) and 2-picoline borane complex (0.43 g, 3.88 mmol) and the reaction mixture was stirred at room temperature for 16 h. After completion, the reaction mixture was concentrated in vacuo. The crude material was purified by combi-flash chromatography, by eluting with 14% MeOH in DCM, to afford benzyl 4-((1-(3-bromo-2-(((2,6-dioxopiperidin-3-yl)amino)methyl)phenyl)piperidin-4-yl)methoxy)piperidine-1-carboxylate (Intermediate 598, 4.0 g, 82%) as grey semi-solid. Mass spec m/z 629.1 [M+H]⁺.

### Step 4

### Intermediate 599: benzyl 4-((1-(2-(2,6-dioxopiperidin-3-yl)-1-oxoisoindolin-4-yl)piperidin-4-yl)methoxy)piperidine-1-carboxylate

To a solution of benzyl 4-((1-(3-bromo-2-(((2,6-dioxopiperidin-3-yl)amino)methyl)phenyl)piperidin-4-yl)methoxy)piperidine-1-carboxylate (Intermediate 598, 3.90 g, 6.2 mmol) in 1,4-dioxane (50 mL) was added triethylamine (2.6 mL, 19.0 mmol). The reaction mixture was purged with argon for 15 min then tungsten hexacarbonyl (1.1 g, 3.1 mmol), PdCl₂(dppf) (0.96 g, 1.20 mmol) and Xantphos (0.74 g, 1.20 mmol) were added. The reaction mixture was further purged with argon for 10 min and heated at 100° C. for 16 h. After completion, the reaction mixture was concentrated in vacuo. The crude material was purified by combi-flash chromatography, by eluting with 12% MeOH in DCM, to afford benzyl 4-((1-(2-(2,6-dioxopiperidin-3-yl)-1-oxoisoindolin-4-yl)piperidin-4-yl)methoxy)piperidine-1-carboxylate (Intermediate 599, 2.50 g, 70%) as a brown solid. ¹H NMR (400 MHz, DMSO-d₆) δ ppm 1.30-1.44 (m, 6H), 1.60-1.79 (m, 7H), 1.93-1.99 (m, 1H), 2.54-2.77 (m, 3H), 2.85-2.97 (m, 1H), 3.17-3.29 (m, 3H), 3.36-3.47 (m, 2H), 3.60-3.63 (m, 2H), 4.22-4.29 (m, 1H), 4.37-4.47 (m, 1H), 5.09 (s, 2H), 7.11-7.19 (m, 2H), 7.31-7.46 (m, 6H), 10.98 (br s, 1H). Mass spec m/z 575.2 [M+H]⁺.

### Step 5

### Intermediate 600: 3-(1-oxo-4-(4-((piperidin-4-yloxy)methyl)piperidin-1-yl)isoindolin-2-yl)piperidine-2,6-dione

A solution of benzyl 4-((1-(2-(2,6-dioxopiperidin-3-yl)-1-oxoisoindolin-4-yl)piperidin-4-yl)methoxy)piperidine-1-carboxylate (Intermediate 599, 1.20 g, 2.1 mmol) in 1,1,1-trifluoroacetone (50 mL) was heated at 50° C. for 8 h. The reaction mixture was concentrated in vacuo. Then residue was dissolved in acetonitrile (10 mL) and cooled to 0° C. for the addition of sodium bicarbonate (0.89 g, 10.0 mmol). The reaction mixture was stirred at room temperature for 1 h then concentrated in vacuo to one-third volume. The resultant precipitate was collected by filtration and dried in vacuo to afford 3-(1-oxo-4-(4-((piperidin-4-yloxy)methyl)piperidin-1-yl)isoindolin-2-yl)piperidine-2,6-dione (Intermediate 600, 0.46 g, 30%) as a grey solid. ¹H NMR (400 MHz, DMSO-d₆) δ ppm 1.17-1.43 (m, 5H), 1.61-1.68 (m, 1H), 1.71-1.79 (m, 6H), 1.97-1.96 (m, 2H), 2.40-2.45 (m, 3H), 2.55-2.64 (m, 2H), 2.64-2.77 (m, 2H), 2.87-2.95 (m, 3H), 4.25-4.30 (m, 1H), 4.39-4.44 (m, 1H), 5.05-5.12 (m, 1H), 7.16 (d, J=7.83 Hz, 1H), 7.29 (d, J=7.04 Hz, 1H), 7.36-7.48 (m, 1H), two exchangeable protons were not observed. Mass spec m/z 441.0 [M+H]⁺.

### Step 6

### Example 147: 6-(4-((1-(2-(2,6-dioxopiperidin-3-yl)-1-oxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)nicotinamide

To a solution of 3-(1-oxo-4-(4-((piperidin-4-yloxy)methyl)piperidin-1-yl)isoindolin-2-yl)piperidine-2,6-dione (Intermediate 600, 0.44 g, 0.99 mmol) and tert-butyl (R)-6-(6-fluoronicotinamido)-2-(1-methylpyrrolidin-2-yl)-1H-pyrrolo[3,2-c]pyridine-1-carboxylate (Intermediate 562, 0.43 g, 0.99 mmol) in dimethyl sulfoxide (1 mL) was added N,N-diisopropylethylamine (0.69 mL, 3.99 mmol) and the reaction mixture was heated at 120° C. for 16 h. After completion, the reaction mixture was concentrated in vacuo. The residue was diluted with ice cold water (100 mL), the resultant precipitate was collected by filtration, washed with n-pentane (50 mL), and dried in vacuo. The crude material was purified by preparative HPLC (Method B) to afford 6-(4-((1-(2-(2,6-dioxopiperidin-3-yl)-1-oxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)nicotinamide (Example 147, 0.13 g, 17%) as an off-white solid. ¹H NMR (400 MHz, DMSO-d₆) δ ppm 1.34-1.40 (m, 2H), 1.46-1.48 (m, 2H), 1.74-2.00 (m, 10H), 2.13-2.15 (m, 1H), 2.16 (s, 3H), 2.22-2.28 (m, 1H), 2.48-2.50 (m, 2H), 2.54-2.60 (m, 2H), 2.70-2.76 (m, 1H), 2.84-2.95 (m, 1H), 3.33-3.45 (m, 6H), 3.54-3.63 (m, 1H), 3.99-4.06 (m, 2H), 4.31-4.35 (m, 1H), 4.40-4.45 (m, 1H), 5.01-5.11 (m, 1H), 6.37 (s, 1H), 6.89 (d, J=8.24 Hz, 1H), 7.11-7.17 (m, 1H), 7.29 (d, J=8.48 Hz, 1H), 7.40-7.44 (m, 1H), 8.13-8.16 (m, 2H), 8.47 (s, 1H), 8.78 (s, 1H), 10.24 (br s, 1H), 10.96 (br s, 1H), 11.32 (br s, 1H). Mass spec m/z 760.3 [M+H]⁺.

### Example 148 was Synthesised Following Scheme 144

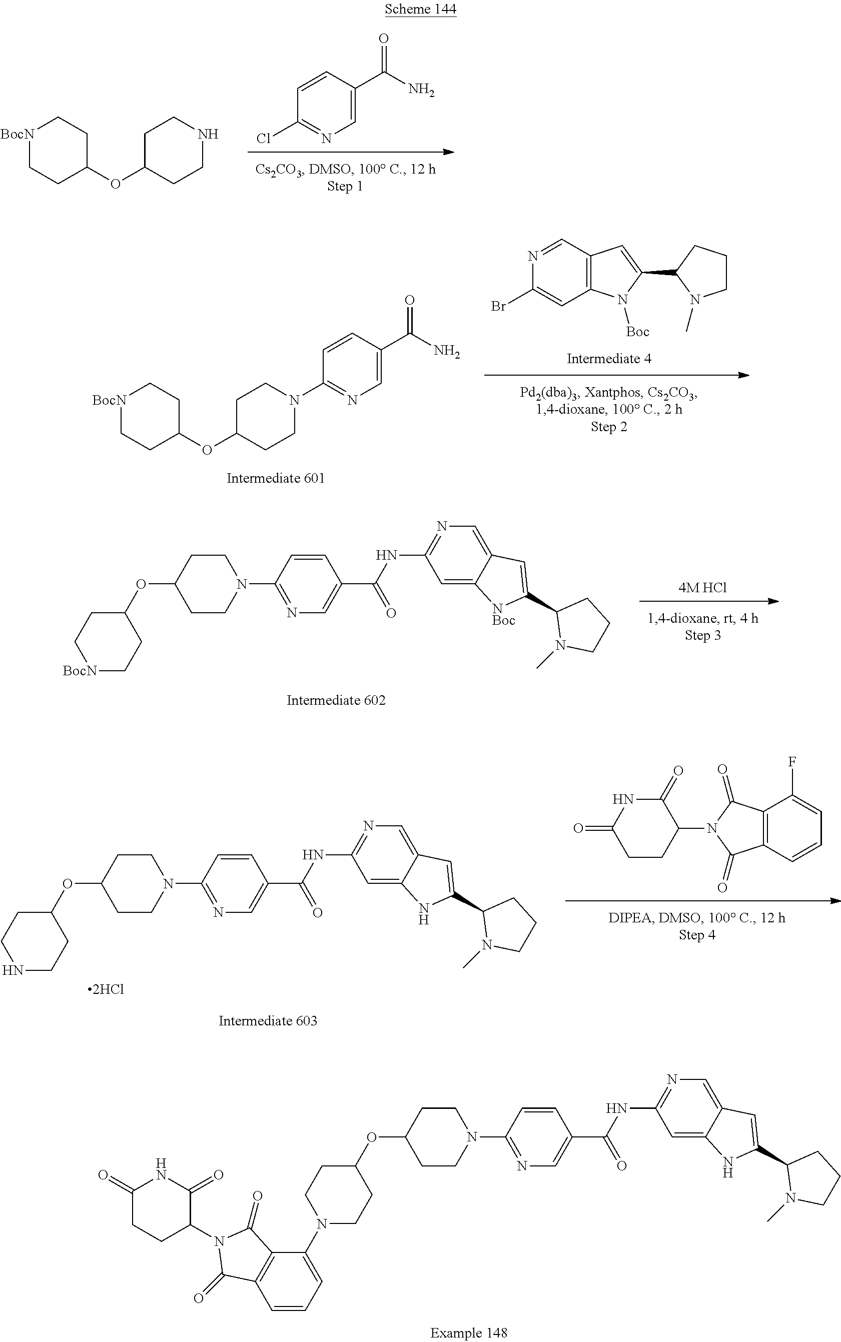

### Step 1

### Intermediate 601: tert-butyl 4-((1-(5-carbamoylpyridin-2-yl)piperidin-4-yl)oxy)piperidine-1-carboxylate

To a solution of 6-chloronicotinamide (CAS No. 6271-78-9, 3.30 g, 21.1 mmol) and tert-butyl 4-(piperidin-4-yloxy)piperidine-1-carboxylate (CAS No. 962-664-6, 5.0 g, 17.6 mmol) in dimethyl sulfoxide (50 mL) was added cesium carbonate (17.3 g, 52.74 mmol) and the reaction mixture was heated at 100° C. for 12 h. The reaction mixture was then diluted with water (100 mL) and extracted with ethyl acetate (2×100 mL). The combined organic layers were dried over anhydrous Na₂SO₄, filtered and concentrated in vacuo. The crude material was purified by combi-flash chromatography, by eluting with 30% ethyl acetate in heptane, to afford tert-butyl 4-((1-(5-carbamoylpyridin-2-yl)piperidin-4-yl)oxy)piperidine-1-carboxylate (Intermediate 601, 3.0 g, 42%) as a white solid. ¹H NMR (400 MHz, DMSO-d₆) δ ppm 1.27-1.38 (m, 13H), 1.71-1.87 (m, 4H), 2.96-3.05 (s, 2H), 3.18-3.26 (m, 2H), 3.59-3.70 (m, 4H), 4.00-4.05 (m, 2H), 6.82 (d, J=8.80 Hz, 1H), 7.09 (s, 1H), 7.71 (s, 1H), 7.91 (d, J=8.80 Hz, 1H), 8.58 (s, 1H). Mass spec m/z 405.4 [M+H]⁺.

### Step 2

### Intermediate 602: tert-butyl (R)-6-(6-(4-((1-(tert-butoxycarbonyl)piperidin-4-yl)oxy)piperidin-1-yl)nicotinamido)-2-(1-methylpyrrolidin-2-yl)-1H-pyrrolo[3,2-c]pyridine-1-carboxylate

To a solution of tert-butyl 4-((1-(5-carbamoylpyridin-2-yl)piperidin-4-yl)oxy)piperidine-1-carboxylate (Intermediate 601, 0.42 g, 1.05 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.5 g, 1.31 mmol) followed by cesium carbonate (1.29 g, 3.94 mmol). The reaction mixture was purged with argon for 15 min, then Pd₂(dba)₃ (0.18 g, 0.19 mmol) and Xantphos (0.23 g, 0.39 mmol) were added. The reaction mixture was further purged with argon for 10 min and heated at 100° C. for 2 h. The reaction mixture was then concentrated in vacuo and the crude material was purified by combi-flash chromatography, by eluting with 90% ethyl acetate in heptane, to afford tert-butyl (R)-6-(6-(4-((1-(tert-butoxycarbonyl)piperidin-4-yl)oxy)piperidin-1-yl)nicotinamido)-2-(1-methylpyrrolidin-2-yl)-1H-pyrrolo[3,2-c]pyridine-1-carboxylate (Intermediate 602, 0.6 g, 65%) as a white solid. ¹H NMR (400 MHz, DMSO-d₆) δ ppm 1.38-1.46 (m, 12H), 1.60-1.80 (m, 16H), 1.81-1.89 (m, 2H), 2.31-2.40 (m, 4H), 2.98-3.15 (s, 4H), 3.60-3.74 (m, 5H), 3.88-3.95 (m, 1H), 4.01-4.07 (m, 2H), 6.73 (s, 1H), 6.88 (d, J=8.40 Hz, 1H), 8.13 (d, J=8.40 Hz, 1H), 8.57 (s, 1H), 8.78 (s, 1H), 8.90 (s, 1H), 10.48 (s, 1H). Mass spec m/z 704.5 [M+H]⁺.

### Step 3

### Intermediate 603: (R)—N-(2-(1-methylpyrrolidin-2-yl)-1H-pyrrolo[3,2-c]pyridin-6-yl)-6-(4-(piperidin-4-yloxy)piperidin-1-yl)nicotinamide dihydrochloride

To a cooled (0° C.) solution of tert-butyl (R)-6-(6-(4-((1-(tert-butoxycarbonyl)piperidin-4-yl)oxy)piperidin-1-yl)nicotinamido)-2-(1-methylpyrrolidin-2-yl)-1H-pyrrolo[3,2-c]pyridine-1-carboxylate (Intermediate 602, 0.50 g, 0.71 mmol) in 1,4-dioxane (5 mL) was added 4M hydrochloric acid in 1,4-dioxane (5 mL, 20.0 mmol) and the reaction mixture was stirred at room temperature for 4 h. The reaction mixture was then concentrated in vacuo to afford (R)—N-(2-(1-methylpyrrolidin-2-yl)-1H-pyrrolo[3,2-c]pyridin-6-yl)-6-(4-(piperidin-4-yloxy)piperidin-1-yl)nicotinamide dihydrochloride (Intermediate 603, 0.80 g) as an off-white solid, which was used for the next step without further purification. ¹H NMR (400 MHz, DMSO-d₆), δ ppm 1.05-1.13 (m, 1H), 1.42-1.59 (m, 5H), 1.65-1.77 (m, 2H), 1.84-2.00 (m, 4H), 2.16-2.18 (m, 2H), 2.64-2.67 (m, 1H), 2.80-2.83 (m, 2H), 2.90-3.01 (m, 2H), 3.13-3.17 (m, 2H), 3.20-3.31 (m, 1H), 3.34-3.41 (m, 1H), 3.67-3.82 (m, 3H), 4.06-4.08 (m, 2H), 4.74-4.76 (m, 1H), 5.75 (s, 1H), 7.16-7.22 (m, 1H), 7.27-7.32 (m, 1H), 8.27-8.32 (m, 1H), 8.40-8.46 (m, 1H), 8.96 (s, 1H), 9.09 (s, 1H), 12.20 (s, 1H), 13.40 (br s, 1H). Mass spec m/z 504.3 [M+H]⁺.

### Step 4

### Example 148: 6-(4-((1-(2-(2,6-dioxopiperidin-3-yl)-1,3-dioxoisoindolin-4-yl)piperidin-4-yl)oxy)piperidin-1-yl)-N-(2-((R)-1-methylpyrrolidin-2-yl)-1H-pyrrolo[3,2-c]pyridin-6-yl)nicotinamide

To a solution of (R)—N-(2-(1-methylpyrrolidin-2-yl)-1H-pyrrolo[3,2-c]pyridin-6-yl)-6-(4-(piperidin-4-yloxy)piperidin-1-yl)nicotinamide hydrochloride (Intermediate 603, 0.50 g, 0.99 mmol) in dimethyl sulfoxide (10 mL) were added N,N-diisopropylethylamine (0.69 mL, 3.97 mmol) followed by 2-(2,6-dioxopiperidin-3-yl)-4-fluoroisoindoline-1,3-dione (CAS No. 835616-60-9, 0.27 g, 0.99 mmol) and the reaction mixture was heated at 100° C. for 12 h. The reaction mixture was then diluted with water (100 mL), the resultant precipitate was collected by filtration and dried in vacuo. The crude material was purified by preparative HPLC (Method A) to afford 6-(4-((1-(2-(2,6-dioxopiperidin-3-yl)-1,3-dioxoisoindolin-4-yl)piperidin-4-yl)oxy)piperidin-1-yl)-N-(2-((R)-1-methylpyrrolidin-2-yl)-1H-pyrrolo[3,2-c]pyridin-6-yl)nicotinamide (Example 148, 0.022 g, 3%) as a yellow solid. ¹H NMR (400 MHz, DMSO-d₆), δ ppm 1.40-1.49 (m, 2H), 1.61-1.70 (m, 2H), 1.75-2.06 (m, 8H), 2.11-2.19 (m, 4H), 2.21-2.28 (m, 1H), 2.57-2.61 (m, 2H), 2.81-2.91 (m, 1H), 3.08-3.17 (m, 3H), 3.28-3.30 (m, 2H), 3.32-3.36 (m, 1H), 3.51-3.56 (m, 2H), 3.71-3.82 (m, 2H) 4.08-4.14 (m, 2H), 5.07-5.12 (m, 1H), 6.37 (s, 1H), 6.98 (d, J=9.2 Hz, 1H), 7.32-7.37 (m, 2H), 7.66-7.70 (m, 1H), 8.14-8.16 (m, 2H), 8.48 (s, 1H), 8.79 (s, 1H), 10.26 (s, 1H), 11.08 (br s, 1H), 11.33 (s, 1H). Mass spec m/z 760.3 [M+H]⁺.

### Example 149 was Synthesised Following Scheme 145

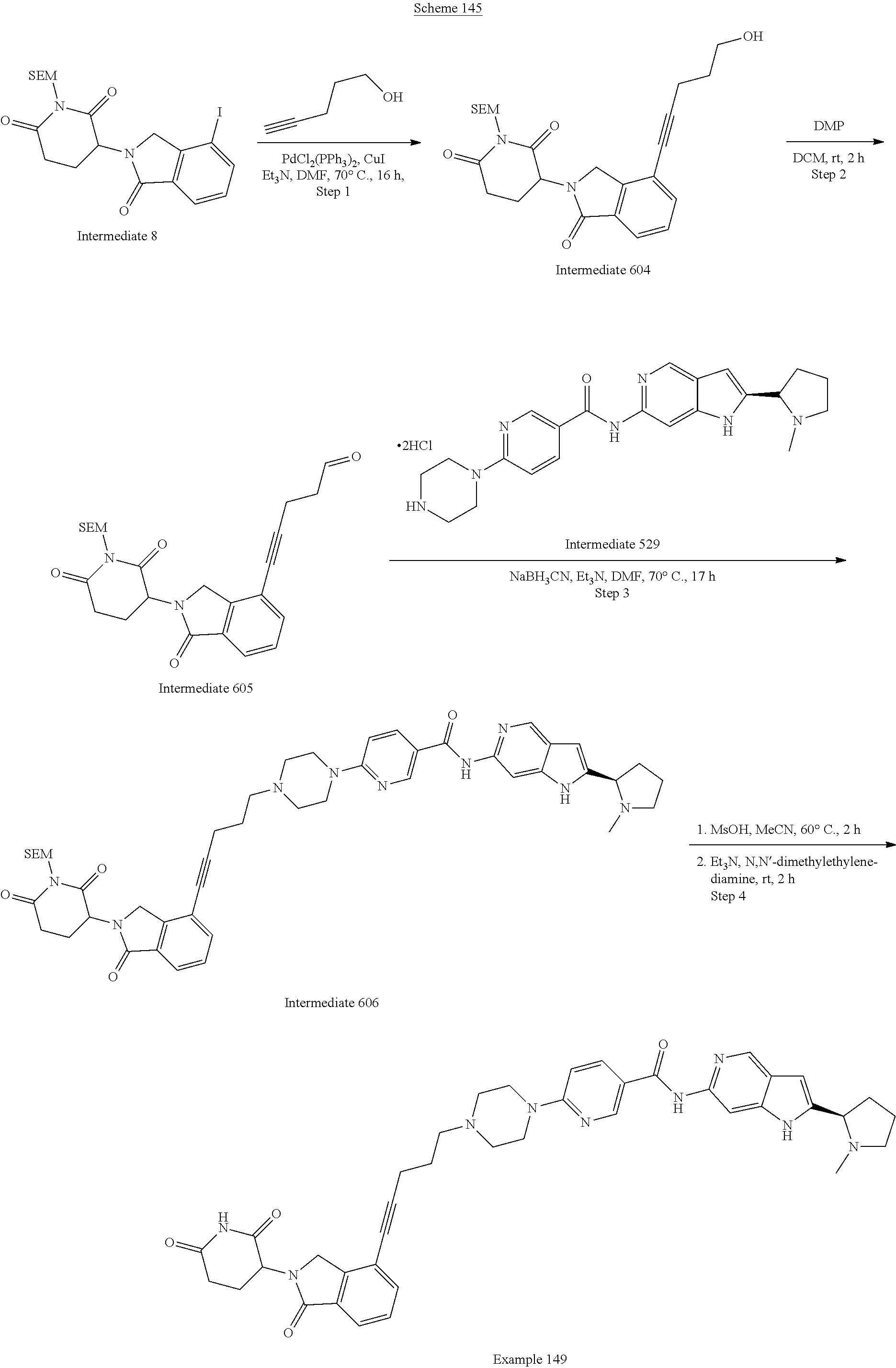

### Step 1

### Intermediate 604: 3-(4-(5-hydroxypent-1-yn-1-yl)-1-oxoisoindolin-2-yl)-1-((2-(trimethylsilyl)ethoxy)methyl)piperidine-2,6-dione

To a cooled (0° C.) solution of 3-(4-iodo-1-oxoisoindolin-2-yl)-1-((2-(trimethylsilyl)ethoxy)methyl)piperidine-2,6-dione (Intermediate 8, 2.0 g, 4.0 mmol) in dimethylformamide (8 mL) were added pent-4-yn-1-ol (CAS No. 5390-04-5, 0.50 g, 6.0 mmol) followed by triethylamine (21.0 mL, 150.0 mmol). The reaction mixture was purged with argon for 15 min then copper (I) iodide (0.07 g, 0.40 mmol) and PdCl₂(PPh₃)₂ (0.29 g, 0.40 mmol) were added. The reaction mixture was purged with argon for another 10 min and heated at 70° C. for 16 h. After completion, the reaction mixture was diluted with water (100 mL) and extracted with ethyl acetate (150 mL). The organic layer was separated, dried over anhydrous Na₂SO₄, filtered, and concentrated in vacuo. The crude material was purified by combi-flash chromatography, by eluting with 50% ethyl acetate in heptane, to afford 3-(4-(5-hydroxypent-1-yn-1-yl)-1-oxoisoindolin-2-yl)-1-((2-(trimethylsilyl)ethoxy)methyl)piperidine-2,6-dione (Intermediate 604, 1.47 g, 81%) as an off-white solid. ¹H NMR (400 MHz, DMSO-d₆) δ ppm −0.07 (s, 9H), 0.81-0.86 (m, 2H), 1.69-1.98 (m, 2H), 2.01-2.08 (m, 1H), 2.43-2.48 (m, 1H), 2.73-2.81 (m, 1H), 3.01-3.07 (m, 1H), 3.50-3.55 (m, 4H), 3.46-3.58 (m, 2H), 4.22-4.28 (m, 1H), 4.44-4.49 (m, 1H), 4.54-4.58 (m, 1H), 5.01-5.09 (m, 2H), 5.25-5.30 (m, 1H), 7.50-7.58 (m, 1H), 7.61-7.67 (m, 1H), 7.71 (d, J=8.25 Hz, 1H).

### Step 2

### Intermediate 605: 5-(2-(2,6-dioxo-1-((2-(trimethylsilyl)ethoxy)methyl)piperidin-3-yl)-1-oxoisoindolin-4-yl)pent-4-ynal

To a cooled (0° C.) solution of 3-(4-(5-hydroxypent-1-yn-1-yl)-1-oxoisoindolin-2-yl)-1-((2-(trimethylsilyl)ethoxy)methyl)piperidine-2,6-dione (Intermediate 604, 0.70 g, 1.53 mmol) in dichloromethane (7 mL) was added Dess-Martin periodinane (1.0 g, 2.30 mmol) and the reaction mixture was stirred at room temperature for 2 h. After completion, the reaction mixture was diluted with water (50 mL) and extracted with ethyl acetate (150 mL). The organic layer was separated, dried over anhydrous Na₂SO₄, filtered and concentrated in vacuo. The crude material was purified by combi-flash chromatography, by eluting with 50% ethyl acetate in heptane, to afford 5-(2-(2,6-dioxo-1-((2-(trimethylsilyl)ethoxy)methyl)piperidin-3-yl)-1-oxoisoindolin-4-yl)pent-4-ynal (Intermediate 605, 0.55 g, 79%) as a semi solid. ¹H NMR (400 MHz, DMSO-d₆) δ ppm −0.02 (s, 9H), 0.81-0.88 (m, 2H), 1.71-1.80 (m, 1H), 2.01-2.10 (m, 1H), 2.72-2.80 (m, 3H), 3.16-3.26 (m, 2H), 3.50-3.58 (m, 2H), 4.02-4.10 (m, 1H), 4.21-4.29 (m, 1H), 4.44-4.54 (m, 1H), 5.01-5.09 (m, 1H), 5.25-5.30 (m, 1H), 7.48-7.54 (m, 2H), 7.59-7.64 (m, 1H), 7.72 (d, J=7.55 Hz, 1H), 9.71 (s, 1H).

### Step 3

### Intermediate 606: 6-(4-(5-(2-(2,6-dioxo-1-((2-(trimethylsilyl)ethoxy)methyl)piperidin-3-yl)-1-oxoisoindolin-4-yl)pent-4-yn-1-yl)piperazin-1-yl)-N-(2-((R)-1-methylpyrrolidin-2-yl)-1H-pyrrolo[3,2-c]pyridin-6-yl)nicotinamide

To a cooled (0° C.) solution of 5-(2-(2,6-dioxo-1-((2-(trimethylsilyl)ethoxy)methyl)piperidin-3-yl)-1-oxoisoindolin-4-yl)pent-4-ynal (Intermediate 605, 0.3 g, 0.7 mmol) and (R)—N-(2-(1-methylpyrrolidin-2-yl)-1H-pyrrolo[3,2-c]pyridin-6-yl)-6-(piperazin-1-yl)nicotinamide dihydrochloride (Intermediate 529, 1.1 g, 2.7 mmol) in dimethylformamide (3 mL) was added triethylamine (0.4 mL, 3.0 mmol) and the reaction mixture was heated at 70° C. for 1 h. The reaction mixture was then cooled to 0° C., sodium cyanoborohydride (0.1 g, 2.01 mmol) was added and the reaction temperature was again brought to 70° C. and stirred for 16 h. After completion, the reaction mixture was diluted with water (10 mL) and extracted with ethyl acetate (20 mL). The organic layer was separated, dried over anhydrous Na₂SO₄, filtered, and concentrated in vacuo. The crude material was purified by combi-flash chromatography, by eluting with 5% MeOH in DCM, to afford 6-(4-(5-(2-(2,6-dioxo-1-((2-(trimethylsilyl)ethoxy)methyl)piperidin-3-yl)-1-oxoisoindolin-4-yl)pent-4-yn-1-yl)piperazin-1-yl)-N-(2-((R)-1-methylpyrrolidin-2-yl)-1H-pyrrolo[3,2-c]pyridin-6-yl)nicotinamide (Intermediate 606, 0.2 g, 40%) as a semi solid. ¹H NMR (400 MHz, DMSO-d₆) δ ppm −0.06 (s, 9H), 0.79-0.87 (m, 2H), 1.09-1.15 (m, 4H), 1.73-1.97 (m, 4H), 2.02-2.12 (m, 1H), 2.16 (s, 3H), 2.21-2.35 (m, 3H), 2.52-2.63 (m, 4H), 2.70-2.86 (m, 4H), 3.00-3.21 (m, 2H), 3.24-3.29 (m, 1H), 3.32-3.38 (m, 2H), 3.44-3.58 (m, 2H), 3.61-3.69 (m, 2H), 4.22-4.35 (m, 1H), 4.40-4.55 (m, 1H), 4.97-5.12 (m, 2H), 6.37 (s, 1H), 6.87 (d, J=8.69 Hz, 1H), 7.49-7.58 (m, 1H), 7.62-7.69 (m, 1H), 7.73 (d, J=7.55 Hz, 1H), 8.16 (s, 1H), 8.48 (s, 1H), 8.79 (s, 1H), 10.27 (br s, 1H), 11.33 (br s, 1H).

### Step 4

### Example 149: 6-(4-(5-(2-(2,6-dioxopiperidin-3-yl)-1-oxoisoindolin-4-yl)pent-4-yn-1-yl)piperazin-1-yl)-N-(2-((R)-1-methylpyrrolidin-2-yl)-1H-pyrrolo[3,2-c]pyridin-6-yl)nicotinamide

To a cooled (0° C.) solution of 6-(4-(5-(2-(2,6-dioxo-1-((2-(trimethylsilyl)ethoxy)methyl)piperidin-3-yl)-1-oxoisoindolin-4-yl)pent-4-yn-1-yl)piperazin-1-yl)-N-(2-((R)-1-methylpyrrolidin-2-yl)-1H-pyrrolo[3,2-c]pyridin-6-yl)nicotinamide (Intermediate 606, 0.3 g, 0.35 mmol) in acetonitrile (3 mL) was added methanesulfonic acid (0.23 mL, 3.55 mmol) and the reaction mixture was heated at 60° C. for 2 h. The reaction mixture was cooled to room temperature, then N,N′-dimethylethylenediamine (0.15 g, 1.77 mmol) followed by triethylamine (0.99 mL, 7.01 mmol) were added and the mixture was stirred at room temperature for 2 h. The reaction mixture was concentrated in vacuo and the crude material was purified by preparative HPLC (Method A) to afford 6-(4-(5-(2-(2,6-dioxopiperidin-3-yl)-1-oxoisoindolin-4-yl)pent-4-yn-1-yl)piperazin-1-yl)-N-(2-((R)-1-methylpyrrolidin-2-yl)-1H-pyrrolo[3,2-c]pyridin-6-yl)nicotinamide (Example 149, 0.01 g, 4%) as an off-white solid. ¹H NMR (400 MHz, DMSO-d₆) δ ppm 1.80-1.90 (m, 6H), 1.97-2.03 (m, 1H), 2.12-2.14 (m, 1H), 2.16 (s, 3H), 2.23-2.29 (m, 1H), 2.42-2.49 (m, 4H), 2.52-2.62 (m, 4H), 2.87-2.97 (m, 1H), 3.11-15 (m, 1H), 3.22-3.29 (m, 2H), 3.60-3.67 (m, 4H), 4.30-4.35 (m, 1H), 4.45-4.49 (m, 1H), 5.13-5.17 (m, 1H), 6.37 (s, 1H), 6.87 (d, J=9.13 Hz, 1H), 7.53 (t, J=7.63 Hz, 1H), 7.62-7.66 (m, 1H), 7.71 (d, J=7.50 Hz, 1H), 8.14-8.19 (m, 2H), 8.47 (s, 1H), 8.80 (s, 1H), 10.28 (br s, 1H), 11.01 (br s, 1H), 11.33 (br s, 1H). Mass spec m/z 714.3 [M+H]⁺.

### Example 150 was Synthesised Following Scheme 146

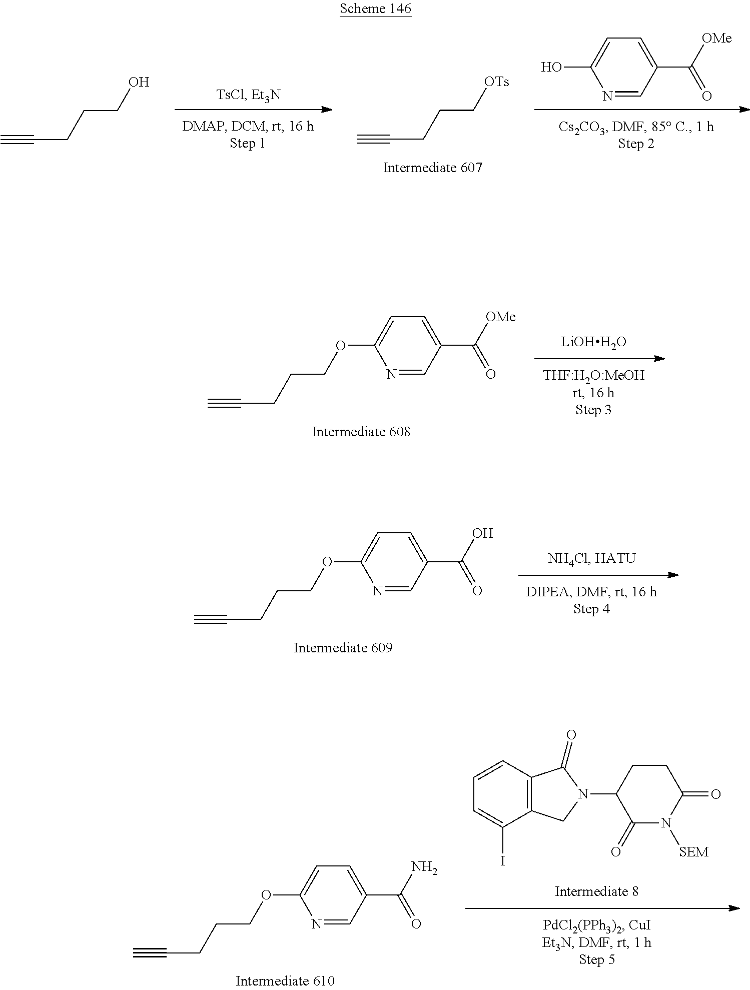

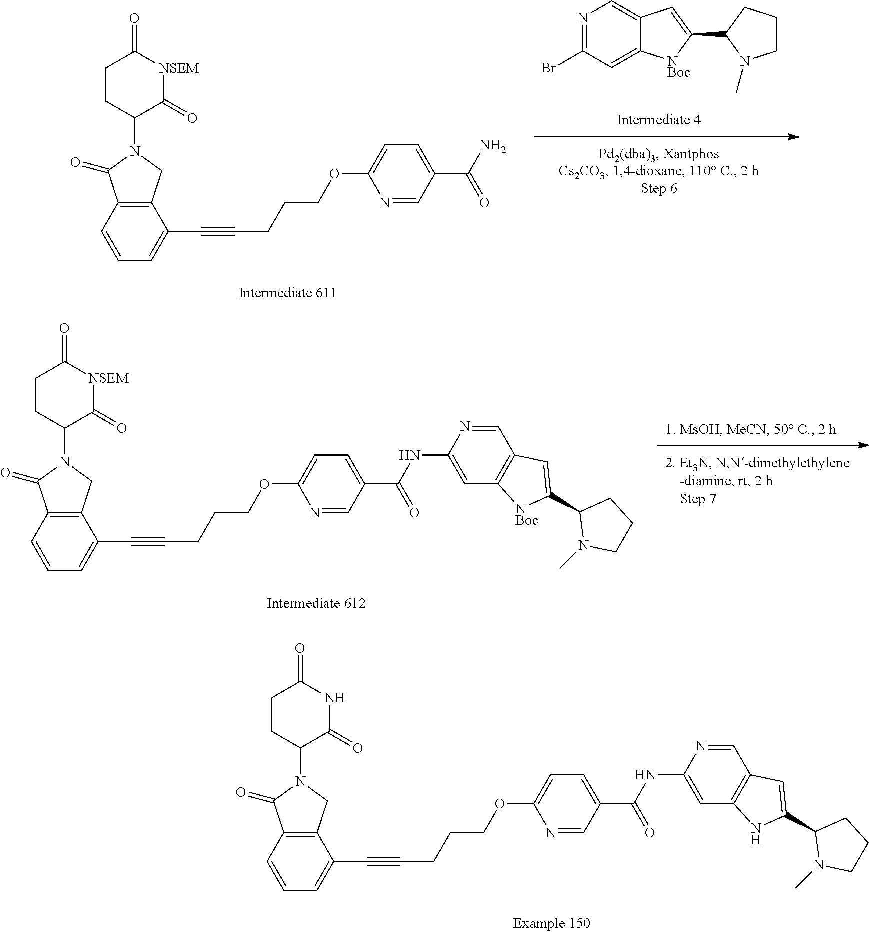

### Step 1

### Intermediate 607: pent-4-yn-1-yl 4-methylbenzenesulfonate

To a cooled (0° C.) solution of pent-4-yn-1-ol (CAS No: 821-09-0, 10.0 g, 118.9 mmol) in dichloromethane (100 mL) were added triethylamine (49.7 mL, 356.6 mmol), 4-toluenesulfonyl chloride (34.0 g, 178.3 mmol) and DMAP (1.53 g, 11.89 mmol) and the reaction mixture was stirred at room temperature for 16 h. After completion, the reaction mixture was quenched with water (200 mL) and extracted with dichloromethane (2×100 mL). The combined organic layers were dried over anhydrous Na₂SO₄ and concentrated in vacuo. The crude material was purified by combi-flash chromatography, by eluting with 15% ethyl acetate in heptane, to afford pent-4-yn-1-yl 4-methylbenzenesulfonate (Intermediate 607, 19.0 g, 67%) as colorless oil. ¹H NMR (400 MHz, DMSO-d₆) δ ppm 1.76-1.72 (m, 2H), 2.17-2.16 (m, 2H), 2.42 (s, 3H), 2.72 (s, 1H), 4.06-4.09 (m, 2H), 7.49 (d, J=8.0 Hz, 2H), 7.79 (d, J=8.0 Hz, 2H). Mass spec m/z 256.3 [M+H+18]+.

### Step 2

### Intermediate 608: methyl 6-(pent-4-yn-1-yloxy)nicotinate

To a solution of methyl 6-hydroxypyridine-3-carboxylate (CAS No: 223788-08-7, 5.0 g, 32.65 mmol) in dimethylformamide (50 mL) was added Cs₂CO₃ (31.83 g, 97.950 mmol) and pent-4-yn-1-yl 4-methylbenzenesulfonate (Intermediate 607, 8.55 g, 35.915 mmol) and the reaction mixture was heated at 85° C. for 1 h. After completion, the reaction mixture was diluted with ethyl acetate (500 mL) and washed with ice cold water (2×500 mL). The organic layer was separated, dried over anhydrous Na₂SO₄ and concentrated in vacuo. The crude material was purified by combi-flash chromatography, by eluting with 12% ethyl acetate in heptane, to afford methyl 6-(pent-4-yn-1-yloxy)nicotinate (Intermediate 608, 1.95 g, 27%) as colourless oil. ¹H NMR (400 MHz, DMSO-d₆) δ ppm 1.87-1.94 (m, 2H), 2.32-2.34 (m, 2H), 2.82 (s, 1H), 3.84 (s, 3H), 4.37-4.41 (m, 2H), 6.92 (d, J=8.8 Hz, 1H), 8.15-8.17 (m, 1H), 8.74 (s, 1H). Mass spec m/z 220.4 [M+H]⁺.

### Step 3

### Intermediate 609: 6-(pent-4-yn-1-yloxy)nicotinic acid

To a solution of methyl 6-(pent-4-yn-1-yloxy)nicotinate (Intermediate 608, 1.85 g, 8.44 mmol) in tetrahydrofuran (10 mL) and water (10 mL) was added lithium hydroxide (0.40 g, 16.9 mmol) and the reaction mixture was stirred at room temperature for 16 h. After completion, the reaction mixture was diluted with water (100 mL) and washed with dichloromethane (2×100 mL). The aqueous phase was adjusted to pH~7 with 1N aqueous HCl solution, the resultant precipitate was collected by filtration and dried in vacuo to afford 6-(pent-4-yn-1-yloxy)nicotinic acid (Intermediate 609, 1.5 g, 87%) as an off-white solid, which was used in the next stage without further purification. ¹H NMR (400 MHz, DMSO-d₆) δ ppm 1.88-1.95 (m, 2H), 2.30-2.35 (m, 2H), 2.81-2.85 (m, 1H), 4.37-4.40 (m, 2H), 6.90 (d, J=8.8 Hz, 1H), 8.14 (dd, J=8.8, 2.4 Hz, 1H), 8.71 (d, J=2.0 Hz, 1H), 13.05 (br s, 1H). Mass spec m/z 204.4 [M−H]⁻.

### Step 4

### Intermediate 610: 6-(pent-4-yn-1-yloxy)nicotinamide

To a stirred solution of 6-(pent-4-yn-1-yloxy)nicotinic acid (Intermediate 609, 1.5 g, 7.3 mmol) in dimethylformamide (8 mL) was added HATU (5.6 g, 15.0 mmol). The reaction mixture was stirred at room temperature for 15 min and then ammonium chloride (2.0 g, 37.0 mmol) and N,N-diisopropylethylamine (4 mL, 22.0 mmol) were added. The reaction mixture was stirred at room temperature for 16 h. After completion, the reaction mixture was poured into ice cold water (60 mL), the resultant precipitate was collected by filtration, washed with water (5 mL), and dried in vacuo to afford 6-(pent-4-yn-1-yloxy)nicotinamide (Intermediate 610, 0.80 g, 54%) as a yellow solid, which was used in the next stage without further purification. ¹H NMR (400 MHz, DMSO-d₆) δ ppm 1.87-1.92 (m, 2H), 2.30-2.33 (m, 2H), 2.81 (s, 1H), 4.34-4.37 (m, 2H), 6.86 (d, J=8.4 Hz, 1H), 7.39 (s, 1H), 7.97 (s, 1H), 8.12 (d, J=8.8 Hz, 1H), 8.66 (s, 1H). Mass spec m/z 205.48 [M+H]⁺.

### Step 5

### Intermediate 611: 6-((5-(2-(2,6-dioxo-1-((2-(trimethylsilyl)ethoxy)methyl)piperidin-3-yl)-1-oxoisoindolin-4-yl)pent-4-yn-1-yl)oxy)nicotinamide

To a solution of 3-(4-iodo-1-oxoisoindolin-2-yl)-1-((2-(trimethylsilyl)ethoxy)methyl)piperidine-2,6-dione (Intermediate 8, 1.47 g, 2.93 mmol) and 6-(pent-4-yn-1-yloxy)nicotinamide (Intermediate 610, 0.75 g, 3.67 mmol) in dimethylformamide (3 mL) was added triethylamine (20 mL, 135.88 mmol). The reaction mixture was purged with argon for 15 min then PdCl₂(PPh₃)₂ (0.40 g, 0.55 mmol) and copper(I) iodide (0.11 g, 0.55 mmol) were added. The reaction mixture was further purged with argon for 10 min and stirred at room temperature for 1 h. After completion of reaction, reaction mixture was poured in ice cold water (70 mL) and extracted with ethyl acetate (2×120 mL). The combined organic layers were dried over anhydrous Na₂SO₄, filtered and concentrated in vacuo. The crude material was purified by combi-flash chromatography, by eluting with 3% MeOH in DCM, to afford 6-((5-(2-(2,6-dioxo-1-((2-(trimethylsilyl)ethoxy)methyl)piperidin-3-yl)-1-oxoisoindolin-4-yl)pent-4-yn-1-yl)oxy)nicotinamide (Intermediate 611, 0.80 g, 38%) as brown liquid. ¹H NMR (400 MHz, DMSO-d₆) δ ppm −0.97 (s, 9H), 0.78-0.87 (m, 2H), 2.03-2.06 (m, 2H), 2.31-2.41 (m, 1H), 2.61-2.67 (m, 2H), 2.71-2.81 (m, 1H), 3.08-3.11 (m, 1H), 3.16 (d, J=6.4 Hz, 1H), 3.47-3.55 (m, 2H), 4.28-4.23 (m, 1H), 4.42-4.47 (m, 3H), 5.00-5.08 (m, 2H), 5.21-5.27 (m, 1H), 6.86 (d, J=8.4 Hz, 1H), 7.39 (bs, 1H), 7.52 (t, J=8.4 Hz, 1H), 7.54 (d, J=7.2 Hz, 1H), 7.72 (d, J=7.2 Hz, 1H), 7.95 (s, 1H), 8.12 (d, J=8.8 Hz, 1H), 8.66 (s, 1H). Mass spec m/z 575.0 [M−H]⁻.

### Step 6

### Intermediate 612: tert-butyl 6-(6-((5-(2-(2,6-dioxo-1-((2-(trimethylsilyl)ethoxy)methyl)piperidin-3-yl)-1-oxoisoindolin-4-yl)pent-4-yn-1-yl)oxy)nicotinamido)-2-((R)-1-methylpyrrolidin-2-yl)-1H-pyrrolo[3,2-c]pyridine-1-carboxylate

To a suspension of tert-butyl (R)-6-bromo-2-(1-methylpyrrolidin-2-yl)-1H-pyrrolo[3,2-c]pyridine-1-carboxylate (Intermediate 4, 0.49 g, 1.30 mmol) and 6-((5-(2-(2,6-dioxo-1-((2-(trimethylsilyl)ethoxy)methyl)piperidin-3-yl)-1-oxoisoindolin-4-yl)pent-4-yn-1-yl)oxy)nicotinamide (Intermediate 611, 0.75 g, 1.30 mmol) in 1,4-dioxane (8 mL) was added cesium carbonate (0.50 g, 2.60 mmol). The reaction mixture was purged with argon for 15 min then Pd₂(dba)₃ (0.17 g, 0.19 mmol) and Xanthphos (0.23 g, 0.39 mmol) were added. The resulting reaction mixture was degassed for another 10 min and stirred at 110° C. for 2 h. After completion of reaction, the reaction was concentrated in vacuo. The crude material was purified by combi-flash chromatography, by eluting with 6% MeOH in DCM, to afford tert-butyl 6-(6-((5-(2-(2,6-dioxo-1-((2-(trimethylsilyl)ethoxy)methyl)piperidin-3-yl)-1-oxoisoindolin-4-yl)pent-4-yn-1-yl)oxy)nicotinamido)-2-((R)-1-methylpyrrolidin-2-yl)-1H-pyrrolo[3,2-c]pyridine-1-carboxylate (Intermediate 612, 0.8 g, 70%) as a yellow solid. ¹H NMR (400 MHz, DMSO-d₆) δ ppm −0.48 (s, 9H), 0.80-0.82 (m, 2H), 1.71-1.75 (m, 9H), 1.98-2.08 (m, 3H), 2.33-2.36 (m, 6H), 2.64-2.79 (m, 5H), 2.88 (s, 1H), 3.02-3.12 (m, 2H), 3.47-3.52 (m, 2H), 3.89-3.91 (m, 1H), 4.24-4.49 (m, 1H), 4.44-4.47 (m, 3H), 4.99-5.06 (m, 2H), 5.23-5.26 (m, 1H), 6.75 (s, 1H), 7.51-7.55 (m, 1H), 7.59-7.66 (m, 1H), 7.71-7.55 (m, 1H), 7.95 (s, 1H), 8.28-8.31 (m, 1H), 8.59-8.65 (m, 1H), 8.83 (s, 1H), 8.91 (s, 1H), 10.78 (s, 1H). Mass spec m/z 875.7 [M+H]⁺.

### Step 7

### Example 150: 6-((5-(2-(2,6-dioxopiperidin-3-yl)-1-oxoisoindolin-4-yl)pent-4-yn-1-yl)oxy)-N-(2-((R)-1-methylpyrrolidin-2-yl)-1H-pyrrolo[3,2-c]pyridin-6-yl)nicotinamide

To a cooled (0° C.) solution of 6-(6-((5-(2-(2,6-dioxo-1-((2-(trimethylsilyl)ethoxy)methyl)piperidin-3-yl)-1-oxoisoindolin-4-yl)pent-4-yn-1-yl)oxy)nicotinamido)-2-((R)-1-methylpyrrolidin-2-yl)-1H-pyrrolo[3,2-c]pyridine-1-carboxylate (Intermediate 612, 0.5 g, 0.57 mmol) in acetonitrile (5 mL) was added methanesulfonic acid (0.58 g, 5.7 mmol) and the reaction mixture was heated at 50° C. for 2 h. The reaction mixture was cooled to room temperature, then N,N′-dimethylethylenediamine (0.32 g, 3.42 mmol) followed by triethylamine (1.21 g, 11.41 mmol) were added and the mixture was stirred at room temperature for 2 h. After completion, the reaction mixture was diluted with water (50 mL), the resultant precipitate was collected by filtration and dried in vacuo. The crude material was purified by preparative HPLC (Method A) to afford 6-((5-(2-(2,6-dioxopiperidin-3-yl)-1-oxoisoindolin-4-yl)pent-4-yn-1-yl)oxy)-N-(2-((R)-1-methylpyrrolidin-2-yl)-1H-pyrrolo[3,2-c]pyridin-6-yl)nicotinamide (Example 150, 0.17 g, 33%) as a white solid. ¹H NMR (400 MHz, DMSO-d₆) δ ppm 1.78-1.91 (m, 3H), 1.98-2.17 (m, 7H), 2.27-2.29 (m, 1H), 2.38-2.45 (m, 1H), 2.60-2.61 (m, 1H), 2.65-2.69 (m, 2H), 2.86-2.91 (m, 1H), 3.14-3.18 (m, 1H), 3.32-3.33 (m, 1H) 4.30-4.34 (m, 1H), 4.43-4.51 (m, 3H), 5.10-5.15 (m, 1H), 6.39 (s, 1H), 6.91 (d, J=8.8 Hz, 1H), 7.53 (t, J=7.6 Hz, 1H), 7.66 (d, J=6.8 Hz, 1H), 7.71 (d, J=7.6 Hz, 1H), 8.17 (s, 1H), 8.30 (dd, J=8.8, 2.4 Hz, 1H), 8.50 (d, J=2.4 Hz, 1H), 8.84 (d, J=2.4 Hz, 1H), 10.55 (br s, 1H), 11.00 (br s, 1H), 11.37 (br s, 1H). Mass spec m/z 646.0 [M−H]⁻.

### Example 151 was Synthesised Following Scheme 147

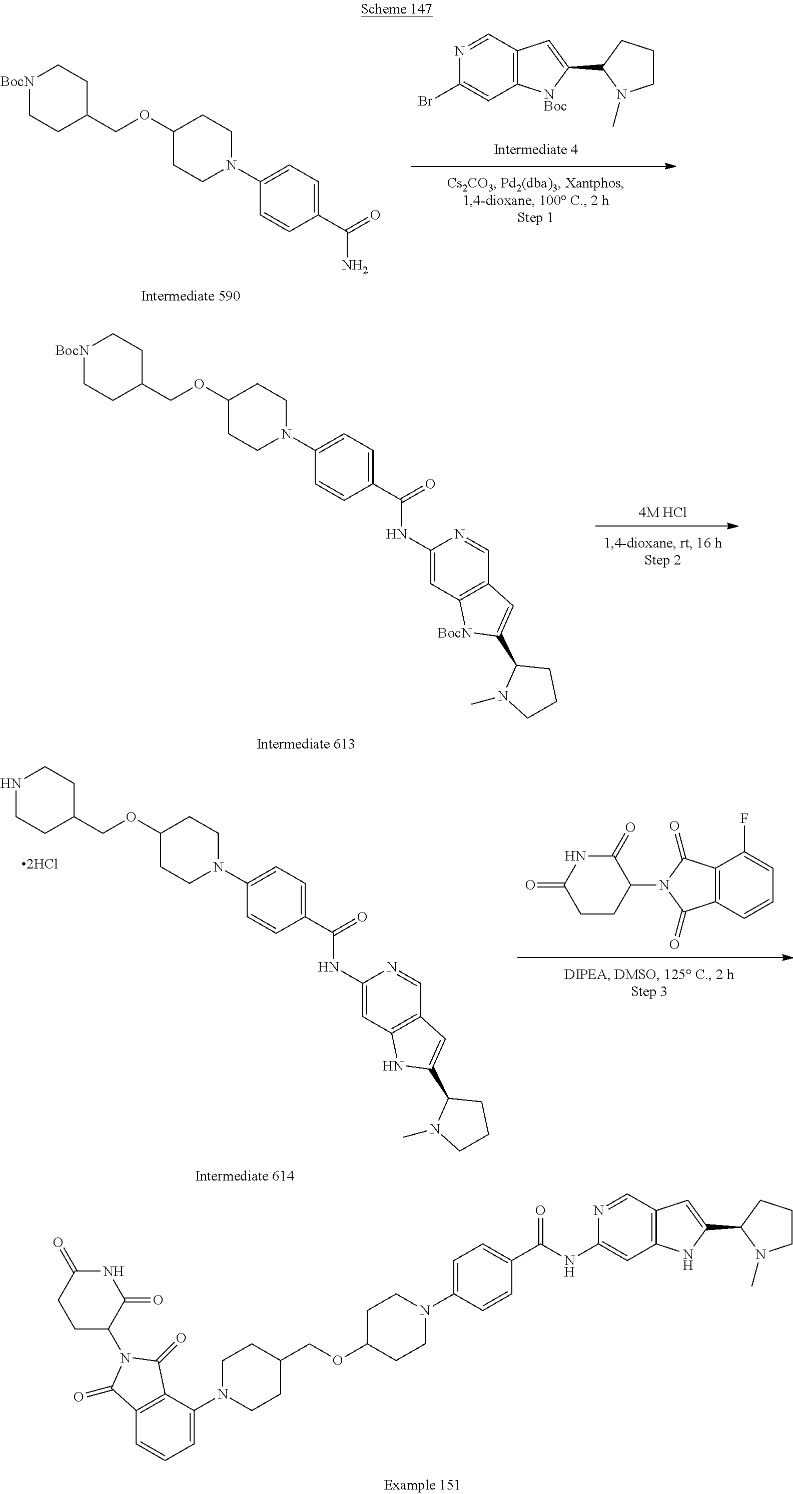

### Step 1

### Intermediate 613: tert-butyl (R)-6-(4-(4-((1-(tert-butoxycarbonyl)piperidin-4-yl)methoxy)piperidin-1-yl)benzamido)-2-(1-methylpyrrolidin-2-yl)-1H-pyrrolo[3,2-c]pyridine-1-carboxylate

To solution of tert-butyl 4-(((1-(4-carbamoylphenyl)piperidin-4-yl)oxy)methyl)piperidine-1-carboxylate (Intermediate 590, 0.9 g, 2.16 mmol) in 1,4-dioxane (2 mL) were added tert-butyl (R)-6-bromo-2-(1-methylpyrrolidin-2-yl)-1H-pyrrolo[3,2-c]pyridine-1-carboxylate (Intermediate 4, 0.9 g, 2.37 mmol) and cesium carbonate (2.13 g, 6.467 mmol). The reaction mixture was purged with argon for 15 min then Pd₂(dba)₃ (0.31 g, 0.32 mmol) and Xanthphos (0.38 g, 0.64 mmol) were added. The reaction mixture was further degassed for another 10 min with argon and heated at 100° C. for 2 h. After completion, the reaction mixture was concentrated in vacuo and the crude material was purified by combi-flash chromatography, by eluting with 3% MeOH in DCM, to afford tert-butyl (R)-6-(4-(4-((1-(tert-butoxycarbonyl)piperidin-4-yl)methoxy)piperidin-1-yl)benzamido)-2-(1-methylpyrrolidin-2-yl)-1H-pyrrolo[3,2-c]pyridine-1-carboxylate (Intermediate 613, 1.1 g, 71%) as a brown solid. ¹H NMR (400 MHz, DMSO-d₆) δ ppm 0.97-1.09 (m, 2H), 1.32-1.41 (m, 10H), 1.45-1.54 (m, 2H), 1.59-1.80 (m, 16H), 1.82-1.92 (m, 2H), 2.29-2.38 (m, 6H), 2.60-2.73 (m, 2H), 3.05-3.17 (m, 4H), 3.45-3.49 (m, 1H), 3.61-3.64 (m, 2H), 3.90-3.98 (m, 3H), 6.73 (s, 1H), 6.97 (d, J=8.4 Hz, 1H), 7.94 (d, J=8.4 Hz, 1H), 8.56 (s, 1H), 8.90 (s, 1H), 10.29 (s, 1H). Mass spec m/z 717.1 [M+H]⁺.

### Step 5

### Intermediate 614: (R)—N-(2-(1-methylpyrrolidin-2-yl)-1H-pyrrolo[3,2-c]pyridin-6-yl)-4-(4-(piperidin-4-ylmethoxy)piperidin-1-yl)benzamide dihydrochloride

To a cooled (0° C.) solution of tert-butyl (R)-6-(4-(4-((1-(tert-butoxycarbonyl)piperidin-4-yl)methoxy)piperidin-1-yl)benzamido)-2-(1-methylpyrrolidin-2-yl)-1H-pyrrolo[3,2-c]pyridine-1-carboxylate (Intermediate 613, 1.0 g, 1.39 mmol) in 1,4-dioxane (20 mL) was added 4M hydrochloric acid in 1,4-dioxane (15 mL) and the reaction mixture was stirred at room temperature for 16 h. After completion, the reaction mixture was concentrated in vacuo and the residue triturated with diethyl ether (20 mL) to afford (R)—N-(2-(1-methylpyrrolidin-2-yl)-1H-pyrrolo[3,2-c]pyridin-6-yl)-4-(4-(piperidin-4-ylmethoxy)piperidin-1-yl)benzamide dihydrochloride (Intermediate 614, 0.9 g) as an off-white solid, which was used for next step without further purification. ¹H NMR (400 MHz, DMSO-d₆) δ ppm 1.32-1.47 (m, 2H), 1.47-1.63 (m, 4H), 1.74-1.98 (m, 5H), 2.14-2.24 (m, 2H), 2.33-2.43 (m, 1H), 2.81 (br s, 3H), 3.15-3.36 (m, 5H), 3.66-3.78 (m, 2H), 4.73-5.05 (m, 7H), 5.75 (s, 1H), 7.10 (d, J=8.29 Hz, 1H), 7.28 (br s, 1H), 8.11 (d, J=8.29 Hz, 1H), 8.23-8.31 (m, 1H), 8.64-8.78 (m, 1H), 8.96-9.09 (m, 2H), 11.82 (br s, 1H), 13.29-13.39 (m, 1H). Mass spec m/z 517.48 [M+H]⁺.

### Step 6

### Example 151: 4-(4-((1-(2-(2,6-dioxopiperidin-3-yl)-1,3-dioxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)benzamide

To a solution of (R)—N-(2-(1-methylpyrrolidin-2-yl)-1H-pyrrolo[3,2-c]pyridin-6-yl)-4-(4-(piperidin-4-ylmethoxy)piperidin-1-yl)benzamide dihydrochloride (Intermediate 614, 0.6 g, 1.08 mmol) in dimethysulfoxide (10 mL) was added N,N-diisopropylethylamine (0.94 mL, 5.42 mmol) and 2-(2,6-dioxopiperidin-3-yl)-4-fluoroisoindoline-1,3-dione (CAS No: 835616-60-9, 0.3 g, 1.08 mmol) and the reaction mixture was heated at 125° C. for 2 h. After completion, the reaction mixture was quenched with ice cold water (100 mL), the resultant precipitate was collected by filtration and dried in vacuo. The crude material was purified by preparative HPLC (Method A) to afford 4-(4-((1-(2-(2,6-dioxopiperidin-3-yl)-1,3-dioxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)benzamide (Example 151, 0.195 g, 23%) as a yellow solid. ¹H NMR (400 MHz, DMSO-d₆) δ ppm 1.35-1.59 (m, 4H), 1.66-1.97 (m, 8H), 1.98-2.08 (m, 1H), 2.09-2.20 (m, 4H), 2.21-2.30 (m, 1H), 2.55-2.64 (m, 2H), 2.82-2.94 (m, 3H), 3.06-3.18 (m, 3H), 3.30-3.33 (m, 1H), 3.35-3.42 (m, 2H), 3.53 (m, 1H), 3.60-3.75 (m, 4H), 5.09 (m, 1H), 6.37 (s, 1H), 6.98 (d, J=9.01 Hz, 2H), 7.33 (t, J=7.88 Hz, 2H), 7.67 (dd, J=8.38, 7.25 Hz, 1H), 7.95 (d, J=8.88 Hz, 2H), 8.17 (s, 1H), 8.47 (s, 1H), 10.04 (br s, 1H), 11.07 (br s, 1H), 11.30 (br s, 1H). Mass spec m/z 773.0 [M+H]⁺.

### Example 152 was Synthesized Following Scheme 148

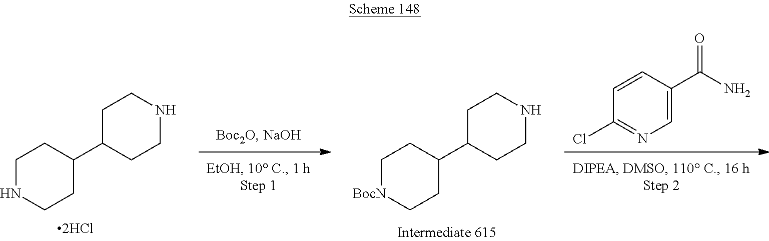 [SMILES: C1CC(C2CCNCC2)CCN1 ; CC(C)(C)OC(=O)N1CCC(C2CCNCC2)CC1 ; NC(=O)c1ccc(Cl)nc1]

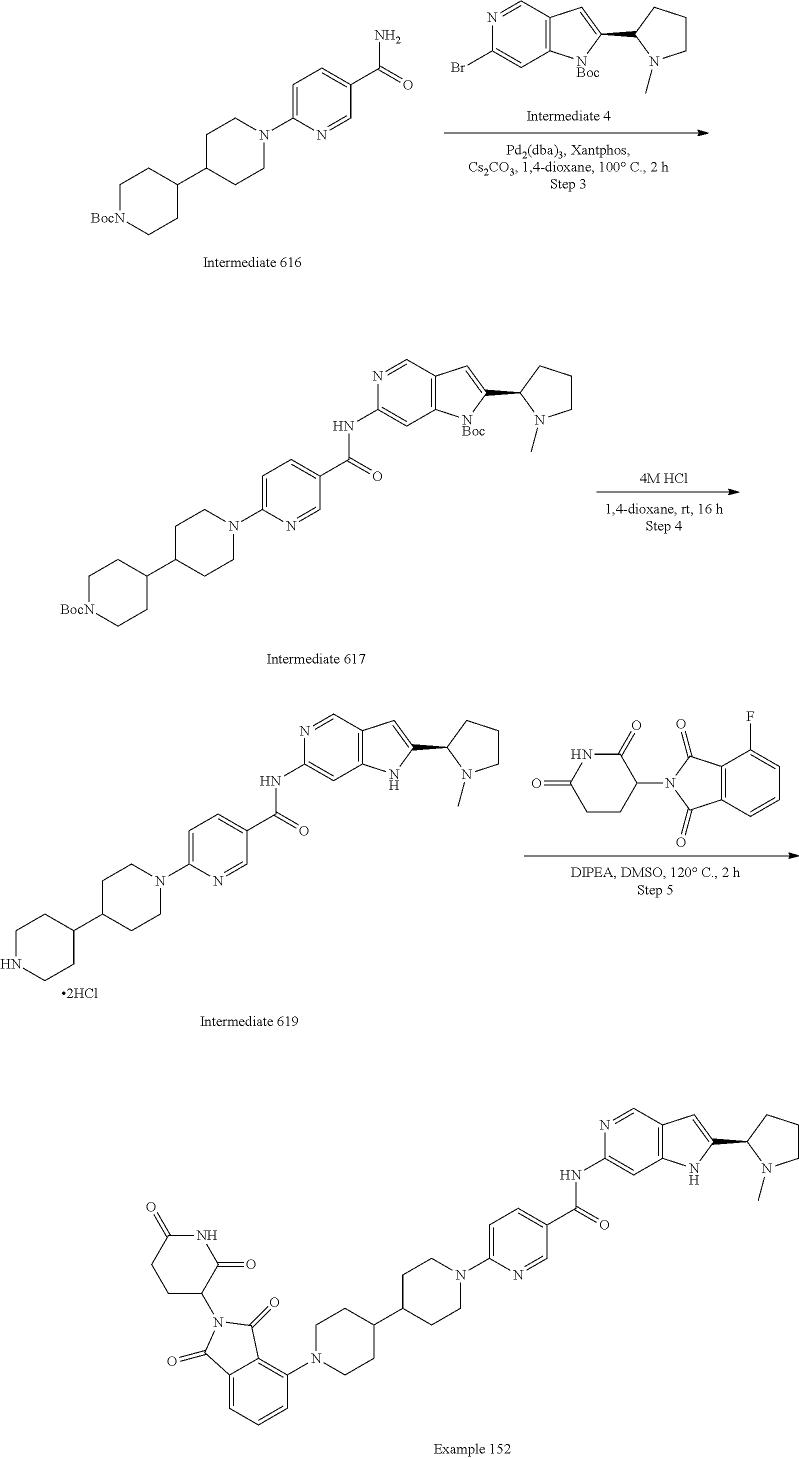

### Step 1

### Intermediate 615: tert-butyl [4,4′-bipiperidine]-1-carboxylate

To a cooled (0° C.) solution of 4-(4-piperidyl)piperidine dihydrochloride (CAS No: 78619-84-8, 11.0 g, 46 mmol) in ethanol (60 mL) were added 2N sodium hydroxide (60 mL), followed by di-tert-butyl dicarbonate (5.0 g, 23 mmol) and the reaction mixture was stirred at 10° C. for 1 h. After completion, reaction mixture was concentrated in vacuo, diluted with saturated aqueous sodium chloride solution (200 mL) and extracted with ethyl acetate (3×200 mL). The combined organic layers were dried over anhydrous Na₂SO₄, filtered and concentrated in vacuo. The crude material was purified by combi-flash chromatography, by eluting 15% MeOH in DCM, to afford tert-butyl [4,4′-bipiperidine]-1-carboxylate (Intermediate 615, 3.40 g, 55%) as an off-white solid. ¹H NMR (400 MHz, DMSO-d₆) δ ppm 0.93-1.22 (m, 6H), 1.38 (s, 9H), 1.56-1.63 (m, 4H), 2.39-2.46 (m, 2H), 2.53-2.59 (bs, 1H), 2.094-2.97 (m, 2H), 3.17 (s, 2H), 3.90-3.97 (m, 2H). Mass spec m/z 269.18 [M+H]⁺.

### Step 2

### Intermediate 616: tert-butyl 1′-(5-carbamoylpyridin-2-yl)-[4,4′-bipiperidine]-1-carboxylate

To stirred suspension of 6-chloropyridine-3-carboxamide (CAS No: 6271-78-9, 1.0 g, 6.4 mmol) in dimethylformamide (20 mL) was added potassium carbonate (3.5 g, 26 mmol) followed by tert-butyl [4,4′-bipiperidine]-1-carboxylate (Intermediate 615, 1.7 g, 6.4 mmol) and the reaction mixture was heated at 110° C. for 16 h. After completion of the reaction, the mixture was poured into ice cold water, the resultant precipitate was collected by filtration and dried in vacuo to afford tert-butyl 1′-(5-carbamoylpyridin-2-yl)-[4,4′-bipiperidine]-1-carboxylate (Intermediate 616, 1.50 g, 60%) as an off-white solid. ¹H NMR (400 MHz, DMSO-d₆) δ ppm 0.98-1.15 (m, 4H), 1.18-1.26 (m, 1H), 1.31-1.42 (m, 11H), 1.63-1.66 (m, 2H), 1.71-1.75 (m, 2H), 2.59-2.66 (m, 1H), 2.73-2.81 (m, 2H), 3.94-3.97 (m, 2H), 4.41-4.46 (m, 2H), 6.81 (d, J=9.2 Hz, 1H), 7.08 (bs, 1H), 7.71 (bs, 1H), 7.92 (dd, J=9.2, 2.4 Hz, 1H), 8.59 (d, J=2.4 Hz, 1H). Mass spec m/z 389.30 [M+H]⁺.

### Step 3

### Intermediate 617: tert-butyl (R)-6-(6-(1′-(tert-butoxycarbonyl)-[4,4′-bipiperidin]-1-yl)nicotinamido)-2-(1-methylpyrrolidin-2-yl)-1H-pyrrolo[3,2-c]pyridine-1-carboxylate

To a suspension of tert-butyl (R)-6-bromo-2-(1-methylpyrrolidin-2-yl)-1H-pyrrolo[3,2-c]pyridine-1-carboxylate (Intermediate 4, 0.65 g, 1.69 mmol) in 1,4-dioxane (10 mL) were added tert-butyl 1′-(5-carbamoylpyridin-2-yl)-[4,4′-bipiperidine]-1-carboxylate (Intermediate 616, 0.6 g, 1.54 mmol) and cesium carbonate (1.58 g, 4.63 mmol). The reaction mixture was purged with argon for 15 min, then Pd₂(dba)₃ (0.20 g, 0.23 mmol) and Xanthphos (0.28 g, 0.46 mmol) were added. The reaction mixture was purged with argon for another 10 min and heated at 100° C. for 2 h. After completion, the reaction mixture was concentrated in vacuo. The crude material was purified by combi-flash chromatography, by eluting with 15% MeOH in DCM, to afford tert-butyl (R)-6-(6-(1′-(tert-butoxycarbonyl)-[4,4′-bipiperidin]-1-yl)nicotinamido)-2-(1-methylpyrrolidin-2-yl)-1H-pyrrolo[3,2-c]pyridine-1-carboxylate (Intermediate 617, 0.80 g, 75%) as a yellow solid. ¹H NMR (400 MHz, DMSO-d₆) δ ppm 1.01-1.24 (m, 5H), 1.39 (s, 9H), 1.63-1.77 (m, 15H), 2.35-2.40 (m, 3H), 2.60-2.61 (m, 2H), 2.79-2.87 (m, 2H), 3.10-3.24 (m, 5H), 3.93-3.98 (m, 3H), 4.44-4.51 (m, 2H), 6.74 (s, 1H), 6.83-6.85 (m, 1H), 8.11-8.13 (m, 1H), 8.56 (s, 1H), 8.78 (s, 1H), 8.89 (s, 1H), 10.22-10.38 (br s, 1H). Mass spec m/z 688.1 [M+H]⁺.

### Step 4

### Intermediate 618: (R)-6-([4,4′-bipiperidin]-1-yl)-N-(2-(1-methylpyrrolidin-2-yl)-1H-pyrrolo[3,2-c]pyridin-6-yl)nicotinamide dihydrochloride

To a cooled (0° C.) solution of tert-butyl (R)-6-(6-(1′-(tert-butoxycarbonyl)-[4,4′-bipiperidin]-1-yl)nicotinamido)-2-(1-methylpyrrolidin-2-yl)-1H-pyrrolo[3,2-c]pyridine-1-carboxylate (Intermediate 617, 0.8 g, 1.16 mmol) in dichloromethane (8 mL) was added 4M hydrochloric acid in 1,4-dioxane (5.8 mL, 23.26 mmol) and the reaction mixture was stirred at room temperature for 16 h. After completion, the reaction mixture was concentrated in vacuo, the residue triturated with diethyl ether (30 mL) and dried in vacuo to afford (R)-6-([4,4′-bipiperidin]-1-yl)-N-(2-(1-methylpyrrolidin-2-yl)-1H-pyrrolo[3,2-c]pyridin-6-yl)nicotinamide dihydrochloride (Intermediate 618, 0.8 g, 92%) as a yellow solid, which was used for next step without further purification. Mass spec m/z 488.2 [M+H]⁺.

### Step 5

### Example 152: 6-(1′-(2-(2,6-dioxopiperidin-3-yl)-1,3-dioxoisoindolin-4-yl)-[4,4′-bipiperidin]-1-yl)-N-(2-((R)-1-methylpyrrolidin-2-yl)-1H-pyrrolo[3,2-c]pyridin-6-yl)nicotinamide

To a cooled (0° C.) solution of (R)-6-([4,4′-bipiperidin]-1-yl)-N-(2-(1-methylpyrrolidin-2-yl)-1H-pyrrolo[3,2-c]pyridin-6-yl)nicotinamide dihydrochloride (Intermediate 618, 0.75 g, 1.43 mmol) and 2-(2,6-dioxopiperidin-3-yl)-4-fluoroisoindoline-1,3-dione (CAS No: 835616-60-9, 0.47 g, 1.71 mmol) in dimethyl sulfoxide (8 mL) was added N,N-diisopropylethylamine (0.75 mL, 4.29 mmol) and the reaction mixture was heated to 120° C. for 2 h. After completion, the reaction mixture was poured into ice cold water (100 mL), resulting in a solid precipitate, which was collected by filtration and dried in vacuo. The crude material was purified by preparative HPLC (Method A) to afford 6-(1′-(2-(2,6-dioxopiperidin-3-yl)-1,3-dioxoisoindolin-4-yl)-[4,4′-bipiperidin]-1-yl)-N-(2-((R)-1-methylpyrrolidin-2-yl)-1H-pyrrolo[3,2-c]pyridin-6-yl)nicotinamide (Example 152, 0.17 g, 11%) as a white solid. ¹H NMR (400 MHz, DMSO-d₆) δ ppm 1.13-1.50 (m, 6H), 1.77-1.94 (m, 7H), 2.01-2.06 (m, 1H), 2.13-2.18 (m, 4H), 2.21-2.30 (m, 1H), 2.56-2.67 (m, 2H), 2.81-2.90 (m, 5H), 3.12-3.15 (m, 1H), 3.28-3.30 (m, 1H), 3.72-3.77 (m, 2H), 4.48-4.53 (m, 2H), 5.06-5.11 (m, 1H), 6.37 (s, 1H), 6.87 (d, J=9.2 Hz, 1H), 7.31-7.34 (m, 2H), 7.65-7.69 (m, 1H), 8.12-8.16 (m, 2H), 8.48 (s, 1H), 8.78-8.79 (m, 1H), 10.24 (s, 1H), 11.08 (s, 1H), 11.32. (s, 1H). Mass spec m/z 744.0 [M+H]⁺.

### Example 153 was Synthesised Following Scheme 149

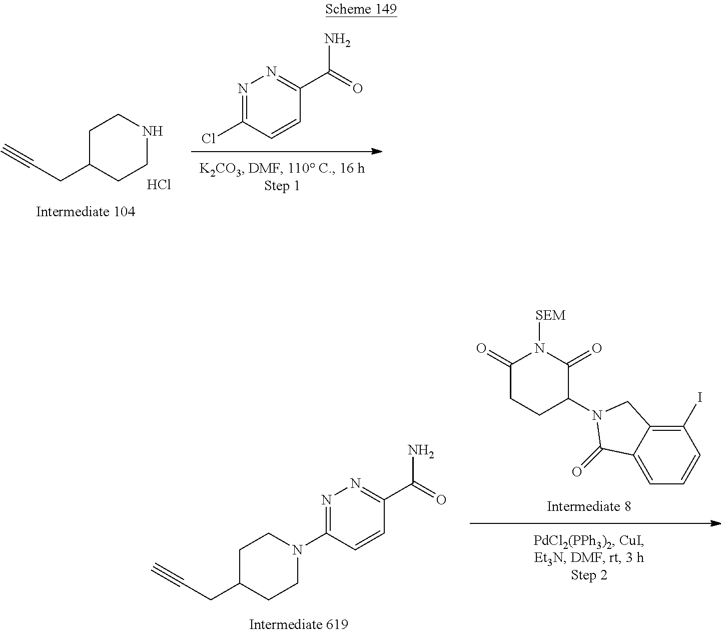

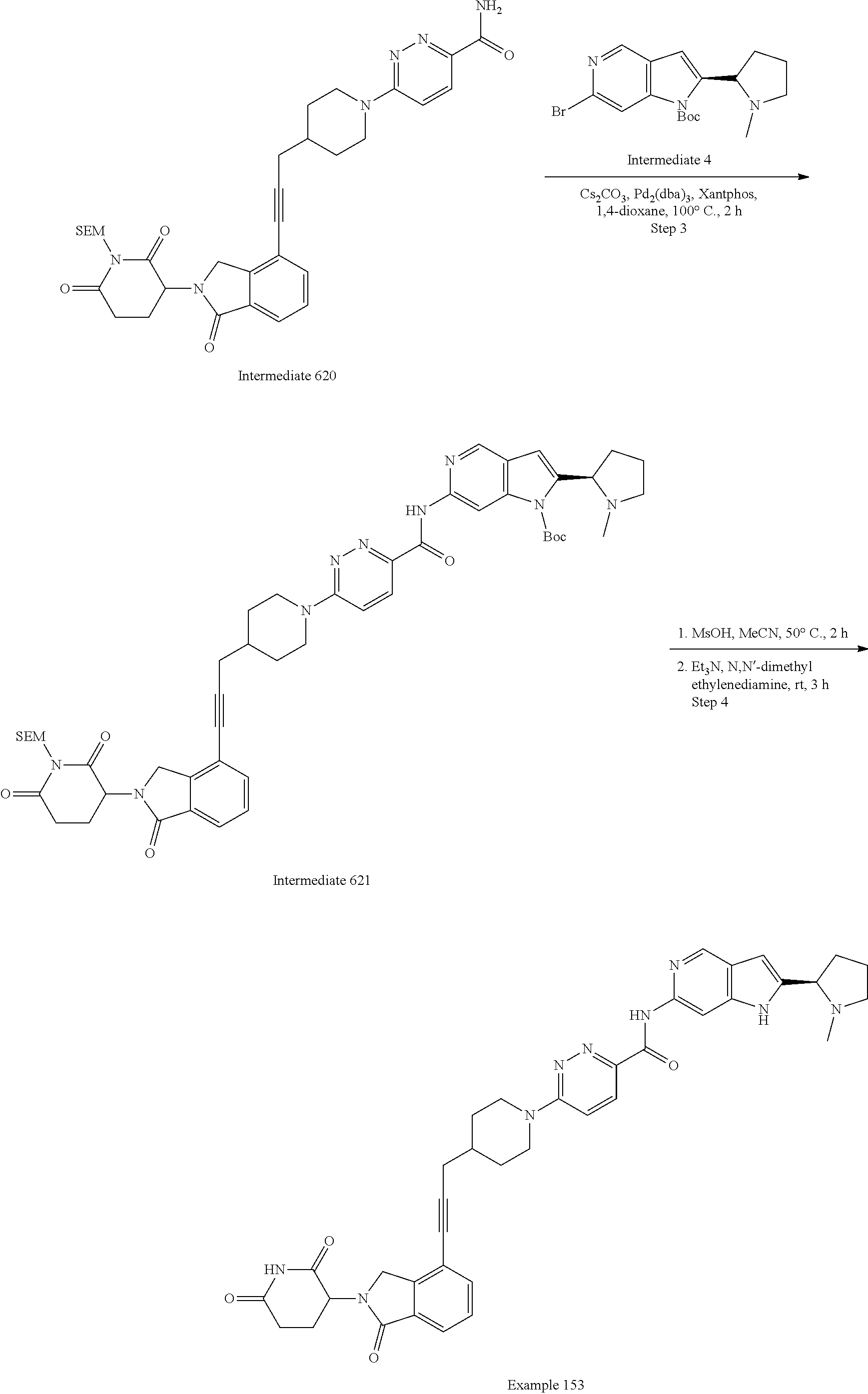

### Step 1

### Intermediate 619: 6-(4-(prop-2-yn-1-yl)piperidin-1-yl)pyridazine-3-carboxamide

To a solution of 4-(prop-2-yn-1-yl)piperidine hydrochloride (Intermediate 104, 1.5 g, 9.50 mmol) and 6-chloropyridazine-3-carboxamide (CAS No: 66346-83-6, 1.4 g, 11.0 mmol) in dimethylformamide (10 mL) was added potassium carbonate (3.9 g, 28.5 mmol) and the reaction mixture was heated at 110° C. for 16 h. After completion, the reaction mixture was quenched with cold water, resulting in a solid precipitate, which was collected by filtration and dried in vacuo to afford 6-(4-(prop-2-yn-1-yl)piperidin-1-yl)pyridazine-3-carboxamide (Intermediate 619, 1.5 g, 64%) as an off-white solid, which was used in the next step without further purification. ¹H NMR (400 MHz, DMSO-d₆) δ ppm 1.17-1.29 (m, 2H), 1.80-1.82 (m, 3H), 2.16-2.18 (m, 2H), 2.80-2.82 (m, 1H), 2.93-3.06 (m, 2H), 4.46-4.57 (m, 2H), 7.33 (d, J=9.5 Hz, 1H), 7.49 (s, 1H), 7.80 (d, J=9.5 Hz, 1H), 8.10 (s, 1H). Mass spec m/z 245.17 [M+H]⁺.

### Step 2

### Intermediate 620: 6-(4-(3-(2-(2,6-dioxo-1-((2-(trimethylsilyl)ethoxy)methyl)piperidin-3-yl)-1-oxoisoindolin-4-yl)prop-2-yn-1-yl)piperidin-1-yl)pyridazine-3-carboxamide

To a solution of 3-(4-iodo-1-oxoisoindolin-2-yl)-1-((2-(trimethylsilyl)ethoxy)methyl)piperidine-2,6-dione (Intermediate 8, 1.02 g, 2.06 mmol) in dimethylformamide (10 mL) were added 6-(4-(prop-2-yn-1-yl)piperidin-1-yl)pyridazine-3-carboxamide (Intermediate 619, 0.50 g, 2.04 mmol) and triethylamine (7.49 g, 73.68 mmol). The reaction mixture was purged with argon for 15 min then PdCl₂(PPh₃)₂ (0.15 g, 0.20 mmol) and copper(I) iodide (0.03 g, 0.20 mmol) were added. The reaction mixture was further purged with argon for 10 min and stirred at room temperature for 3 h. After completion, the reaction mixture was concentrated in vacuo, diluted with water (30 mL) and extracted with ethyl acetate (3×50 mL). The combined organic layers were dried over anhydrous Na₂SO₄, filtered and concentrated in vacuo. The crude material was purified by combi-flash chromatography, by eluting with 100% ethyl acetate, to afford 6-(4-(3-(2-(2,6-dioxo-1-((2-(trimethylsilyl)ethoxy)methyl)piperidin-3-yl)-1-oxoisoindolin-4-yl)prop-2-yn-1-yl)piperidin-1-yl)pyridazine-3-carboxamide (Intermediate 620, 0.7 g, 55%) as a yellow solid. ¹H NMR (400 MHz, DMSO-d₆) δ ppm −0.12-0.05 (m, 9H), 0.75-0.94 (m, 2H), 1.17-1.48 (m, 3H), 1.84-2.14 (m, 4H), 2.28-2.46 (m, 2H), 2.72-2.84 (m, 1H), 2.98-3.16 (m, 3H), 3.45-3.60 (m, 2H), 4.26-4.30 (m, 1H), 4.42-4.61 (m, 3H), 4.96-5.13 (m, 2H), 5.23-5.27 (m, 1H), 7.33 (d, J=9.54 Hz, 1H), 7.46-7.56 (m, 2H), 7.64 (d, J=7.46 Hz, 1H), 7.72 (d, J=7.46 Hz, 1H), 7.81 (d, J=9.54 Hz, 1H), 8.09 (s, 1H). Mass spec m/z 617.38 [M+H]⁺.

### Step 3

### Intermediate 621: tert-butyl 6-(6-(4-(3-(2-(2,6-dioxo-1-((2-(trimethylsilyl)ethoxy)methyl)piperidin-3-yl)-1-oxoisoindolin-4-yl)prop-2-yn-1-yl)piperidin-1-yl)pyridazine-3-carboxamido)-2-((R)-1-methylpyrrolidin-2-yl)-1H-pyrrolo[3,2-c]pyridine-1-carboxylate

To a solution of 6-(4-(3-(2-(2,6-dioxo-1-((2-(trimethylsilyl)ethoxy)methyl)piperidin-3-yl)-1-oxoisoindolin-4-yl)prop-2-yn-1-yl)piperidin-1-yl)pyridazine-3-carboxamide (Intermediate 620, 0.51 g, 0.84 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 0.40 g, 1.05 mmol) and cesium carbonate (1.03 g, 3.15 mmol). The reaction mixture was purged with argon for 15 min then Xanthphos (0.19 g, 0.31 mmol) and Pd₂(dba)₃ (0.14 g, 0.15 mmol) were added. The reaction mixture was further purged with argon for 10 min and heated at 100° C. for 2 h. After completion, the reaction mixture was filtered through a celite bed, the filter pad was washed with ethyl acetate (100 mL) and the filtrate was concentrated in vacuo. The crude material was purified by combi-flash chromatography, by eluting with 80% ethyl acetate in heptane, to afford tert-butyl 6-(6-(4-(3-(2-(2,6-dioxo-1-((2-(trimethylsilyl)ethoxy)methyl)piperidin-3-yl)-1-oxoisoindolin-4-yl)prop-2-yn-1-yl)piperidin-1-yl)pyridazine-3-carboxamido)-2-((R)-1-methylpyrrolidin-2-yl)-1H-pyrrolo[3,2-c]pyridine-1-carboxylate (Intermediate 621, 0.35 g, 36%) as a yellow solid. ¹H NMR (400 MHz, DMSO-d₆) δ ppm −0.36 (s, 9H), 0.73-0.90 (m, 3H), 1.28-1.47 (m, 2H), 1.57-1.81 (m, 6H), 1.94-2.10 (m, 11H), 2.28-2.43 (m, 6H), 2.72-2.85 (m, 1H), 3.04-3.18 (m, 4H), 3.48-3.59 (m, 2H), 3.91-3.94 (m, 1H), 4.27-4.31 (m, 1H), 4.42-4.62 (m, 3H), 5.00-5.10 (m, 1H), 5.22-5.26 (m, 1H), 6.76 (s, 1H), 7.35-7.45 (m, 2H), 7.52 (dd, J=14.93, 7.46 Hz, 1H), 7.63-7.68 (m, 1H), 7.72 (d, J=7.46 Hz, 1H), 7.98 (d, J=9.54 Hz, 1H), 8.59 (s, 1H), 8.95 (s, 1H), 10.27 (s, 1H). Mass spec m/z 915.7 [M]⁺.

### Step 4

### Example 153: 6-(4-(3-(2-(2,6-dioxopiperidin-3-yl)-1-oxoisoindolin-4-yl)prop-2-yn-1-yl)piperidin-1-yl)-N-(2-((R)-1-methylpyrrolidin-2-yl)-1H-pyrrolo[3,2-c]pyridin-6-yl)pyridazine-3-carboxamide

To a solution of tert-butyl 6-(6-(4-(3-(2-(2,6-dioxo-1-((2-(trimethylsilyl)ethoxy)methyl)piperidin-3-yl)-1-oxoisoindolin-4-yl)prop-2-yn-1-yl)piperidin-1-yl)pyridazine-3-carboxamido)-2-((R)-1-methylpyrrolidin-2-yl)-1H-pyrrolo[3,2-c]pyridine-1-carboxylate (Intermediate 621, 0.3 mg, 0.32 mmol) in acetonitrile (5 mL) was added methanesulfonic acid (0.21 mL, 3.27 mmol) and the reaction mixture was heated at 50° C. for 2 h. After cooling to room temperature, N,N′-dimethylethylenediamine (0.39 mL, 3.27 mmol) and triethylamine (0.91 mL, 6.55 mmol) were added and the reaction mixture was stirred at room temperature for 3 h. The reaction mixture was concentrated in vacuo and water (50 mL) added, the resultant precipitate was collected by filtration and dried in vacuo. The crude material was purified by preparative HPLC (Method A) to afford 6-(4-(3-(2-(2,6-dioxopiperidin-3-yl)-1-oxoisoindolin-4-yl)prop-2-yn-1-yl)piperidin-1-yl)-N-(2-((R)-1-methylpyrrolidin-2-yl)-1H-pyrrolo[3,2-c]pyridin-6-yl)pyridazine-3-carboxamide (Example 153, 0.05 g, 25%) as an off-white solid. ¹H NMR (400 MHz, DMSO-d₆) δ ppm 1.29-1.43 (m, 2H), 1.73-2.07 (m, 7H), 2.10-2.20 (m, 4H), 2.22-2.30 (m, 2H), 2.37-2.46 (m, 1H), 2.57-2.69 (m, 3H), 2.85-2.97 (m, 1H), 3.03-3.18 (m, 3H), 4.27-4.36 (m, 1H), 4.42-4.50 (m, 1H), 4.47-4.60 (m, 2H), 5.10-5.15 (m, 1H), 6.40 (s, 1H), 7.45 (d, J=9.6 Hz, 1H), 7.48-7.55 (m, 1H), 7.64 (d, J=6.8 Hz, 1H), 7.71 (d, J=7.4 Hz, 1H), 7.97 (d, J=9.5 Hz, 1H), 8.24 (s, 1H), 8.49 (s, 1H), 10.11 (br s, 1H), 11.01 (br s, 1H), 11.44 (br s, 1H). Mass spec m/z 686.1 [M+H]⁺.

### Example 154 was Synthesised Following Scheme 150

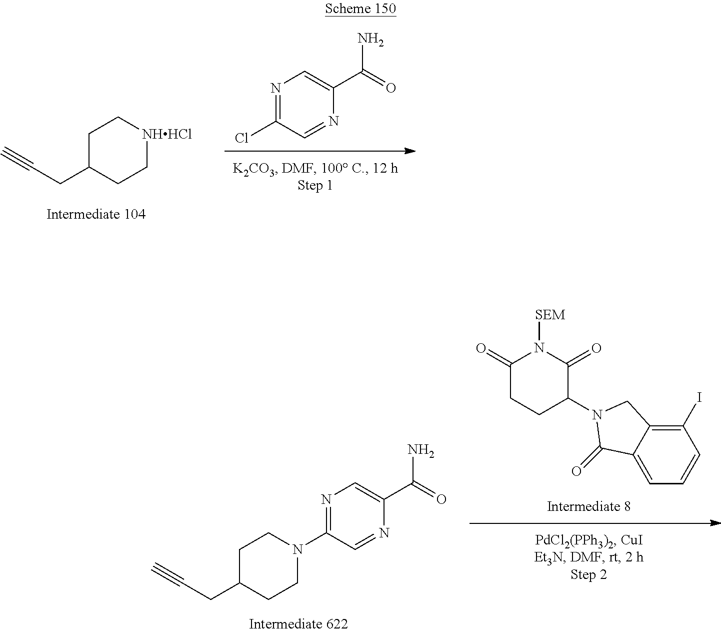

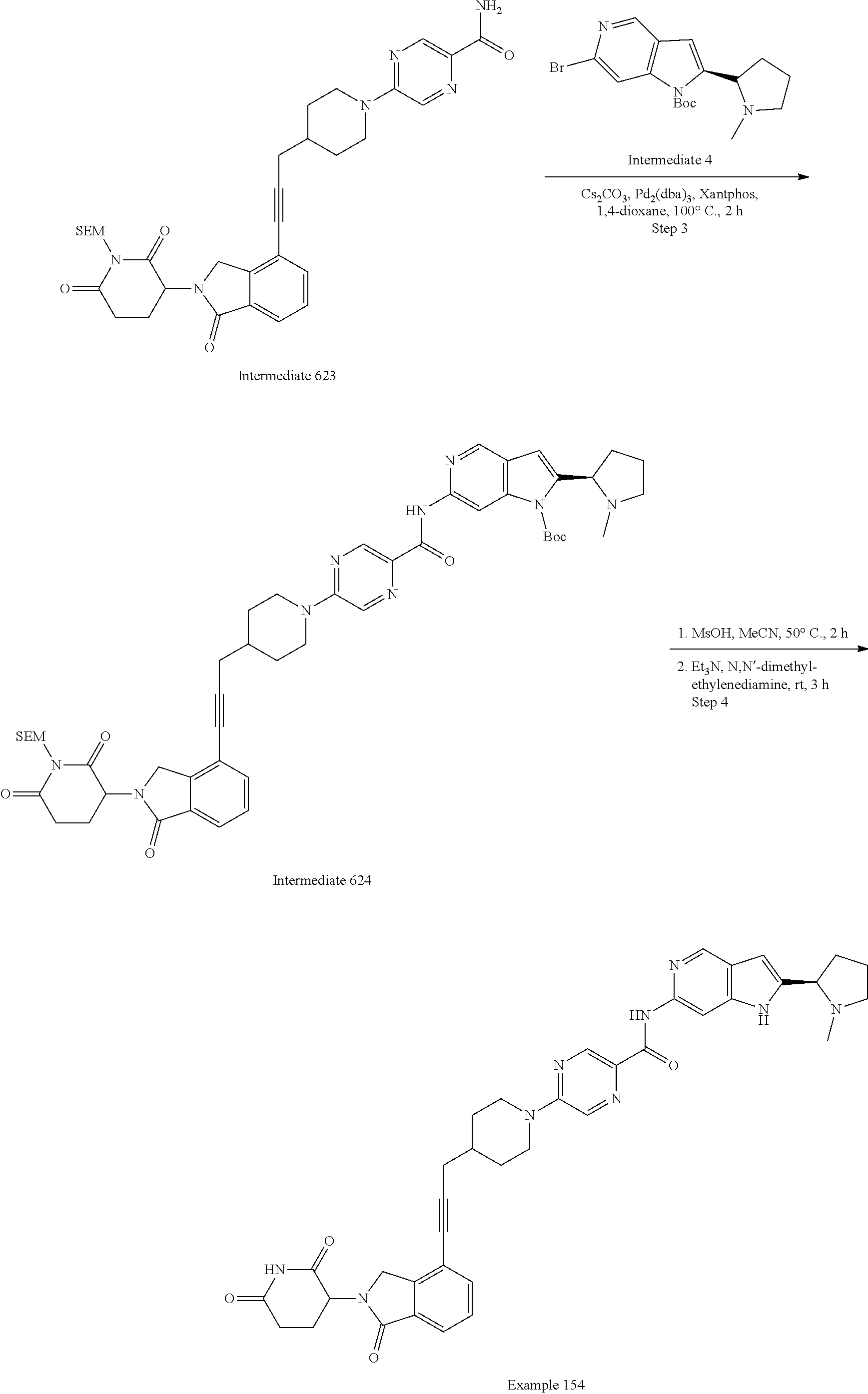

### Step 1

### Intermediate 622: 5-(4-(prop-2-yn-1-yl)piperidin-1-yl)pyrazine-2-carboxamide

To a solution of 4-(prop-2-yn-1-yl)piperidine hydrochloride (Intermediate 104, 1.5 g, 9.40 mmol) in dimethylformamide (20 mL) was added potassium carbonate (3.7 g, 38.0 mmol) and 5-chloropyrazine-2-carboxamide (CAS No: 21279-64-1, 1.5 g, 9.4 mmol) and the reaction mixture was heated at 100° C. for 12 h. After completion, the reaction mixture was diluted with water (100 mL), resulting in a solid precipitate, which was collected by filtration and dried in vacuo to afford 5-(4-(prop-2-yn-1-yl)piperidin-1-yl)pyrazine-2-carboxamide (Intermediate 622, 2.2 g, 96%) as a yellow solid. ¹H NMR (400 MHz, DMSO-d₆) δ ppm 1.98-1.24 (m, 2H), 1.76-1.83 (m, 3H), 2.16-2.18 (m, 2H), 2.83-2.84 (m, 1H), 2.92-2.96 (m, 2H), 4.78-4.51 (m, 2H), 7.35 (m, 1H), 7.71 (s, 1H), 8.27 (d, J=9.5 Hz, 1H), 8.59 (s, 1H).

### Step 2

### Intermediate 623: 5-(4-(3-(2-(2,6-dioxo-1-((2-(trimethylsilyl)ethoxy)methyl)piperidin-3-yl)-1-oxoisoindolin-4-yl)prop-2-yn-1-yl)piperidin-1-yl)pyrazine-2-carboxamide

To a solution of 5-(4-(prop-2-yn-1-yl)piperidin-1-yl)pyrazine-2-carboxamide (Intermediate 622, 0.6 g, 2.0 mmol) in dimethylformamide (5 mL) were added 3-(4-iodo-1-oxoisoindolin-2-yl)-1-((2-(trimethylsilyl)ethoxy)methyl)piperidine-2,6-dione (Intermediate 8, 1.0 g, 2 mmol) and triethylamine (9.1 g, 90 mmol). The reaction mixture was purged with argon for 15 min, then copper(I) iodide (0.05 g, 0.2 mmol) and PdCl₂(PPh₃)₂ (0.2 g, 0.2 mmol) were added. The reaction mixture was further purged with argon for 10 min and stirred at room temperature for 2 h. After completion, the reaction mixture was diluted with water (100 mL) and extracted with ethyl acetate (3×100 mL). The combined organic layers were dried over anhydrous Na₂SO₄ and concentrated in vacuo. The crude material was purified by combi-flash chromatography, by eluting with 95% ethyl acetate in heptane, to afford 5-(4-(3-(2-(2,6-dioxo-1-((2-(trimethylsilyl)ethoxy)methyl)piperidin-3-yl)-1-oxoisoindolin-4-yl)prop-2-yn-1-yl)piperidin-1-yl)pyrazine-2-carboxamide (Intermediate 623, 0.7 g, 50%). ¹H NMR (400 MHz, DMSO-d₆) δ ppm −0.033 (s, 9H), 0.81-0.86 (m, 2H), 1.28-1.32 (m, 3H), 1.89-1.92 (m, 3H), 2.04-2.07 (m, 1H), 2.38-2.47 (m, 2H), 2.77-2.81 (m, 1H), 2.96-3.07 (m, 3H), 3.49-3.55 (m, 2H), 4.25-4.29 (m, 1H), 4.44-4.53 (m, 3H), 5.01-5.09 (m, 2H), 5.23-5.28 (m, 1H), 7.35 (s, 1H), 7.52 (t, J=7.6 Hz, 1H), 7.64 (d, J=7.2 Hz, 1H), 7.69-7.73 (m, 2H), 8.26 (s, 1H), 8.59 (s, 1H). Mass spec m/z 617.34 [M+H]⁺.

### Step 3

### Intermediate 624: tert-butyl 6-(5-(4-(3-(2-(2,6-dioxo-1-((2-(trimethylsilyl)ethoxy)methyl)piperidin-3-yl)-1-oxoisoindolin-4-yl)prop-2-yn-1-yl)piperidin-1-yl)pyrazine-2-carboxamido)-2-((R)-1-methylpyrrolidin-2-yl)-1H-pyrrolo[3,2-c]pyridine-1-carboxylate

To a solution of 5-(4-(3-(2-(2,6-dioxo-1-((2-(trimethylsilyl)ethoxy)methyl)piperidin-3-yl)-1-oxoisoindolin-4-yl)prop-2-yn-1-yl)piperidin-1-yl)pyrazine-2-carboxamide (Intermediate 623, 0.4 g, 0.60 mmol) in 1,4-dioxane (10 mL) were added tert-butyl (R)-6-bromo-2-(1-methylpyrrolidin-2-yl)-1H-pyrrolo[3,2-c]pyridine-1-carboxylate (Intermediate 4, 0.2 g, 0.60 mmol) and cesium carbonate (0.6 g, 2.0 mmol) and the reaction mixture was purged with argon for 15 min. Pd₂(dba)₃ (0.09 g, 0.10 mmol) and Xanthphos (0.1 g, 0.20 mmol) were then added and the reaction mixture was heated at 100° C. for 2 h. After completion, the reaction mixture was concentrated in vacuo. The crude material was purified by combi-flash chromatography, by eluting with 90% ethyl acetate in heptane, to afford tert-butyl 6-(5-(4-(3-(2-(2,6-dioxo-1-((2-(trimethylsilyl)ethoxy)methyl)piperidin-3-yl)-1-oxoisoindolin-4-yl)prop-2-yn-1-yl)piperidin-1-yl)pyrazine-2-carboxamido)-2-((R)-1-methylpyrrolidin-2-yl)-1H-pyrrolo[3,2-c]pyridine-1-carboxylate (Intermediate 624, 0.35 g, 60%) as a brown solid. ¹H NMR (400 MHz, DMSO-d₆) δ ppm −0.30 (s, 9H), 0.77-0.89 (m, 2H), 1.16-1.34 (m, 2H), 1.60-1.76 (m, 14H), 1.91-1.96 (m, 3H), 2.06-2.10 (m, 1H), 2.32-2.40 (m, 7H), 2.78-2.82 (m, 1H), 3.03-3.12 (m, 4H), 3.49-3.55 (m, 2H), 3.90-3.92 (m, 1H), 4.26-4.30 (m, 1H), 4.45-4.49 (m, 1H), 4.57-4.61 (m, 1H), 5.01-5.09 (m, 2H), 5.23-5.28 (m, 1H), 6.75 (s, 1H), 7.50-7.56 (m, 1H), 7.63-7.68 (m, 1H), 7.69-7.71 (m, 1H), 8.41 (s, 1H), 8.56 (s, 1H), 8.76 (s, 1H), 8.95 (s, 1H), 9.91 (s, 1H). Mass spec m/z 915.7 [M]⁺.

### Step 4

### Example 154: 5-(4-(3-(2-(2,6-dioxopiperidin-3-yl)-1-oxoisoindolin-4-yl)prop-2-yn-1-yl)piperidin-1-yl)-N-(2-((R)-1-methylpyrrolidin-2-yl)-1H-pyrrolo[3,2-c]pyridin-6-yl)pyrazine-2-carboxamide

To a solution of 5-(4-(3-(2-(2,6-dioxopiperidin-3-yl)-1-oxoisoindolin-4-yl)prop-2-yn-1-yl)piperidin-1-yl)-N-(2-((R)-1-methylpyrrolidin-2-yl)-1H-pyrrolo[3,2-c]pyridin-6-yl)pyrazine-2-carboxamide (Intermediate 624, 0.35 g, 0.38 mmol) in acetonitrile was added methanesulfonic acid (0.56 g, 5.73 mmol) and the reaction mixture was heated at 50° C. for 2 h. After cooling to room temperature, triethylamine (0.78 g, 7.64 mmol) and N,N′-dimethylethylenediamine (0.27 mL, 2.29 mmol) were then added and the reaction mixture was stirred at room temperature for 3 h. After completion, the reaction mixture was diluted with water (150 mL), the resultant precipitate was collected by filtration and dried in vacuo. The crude material was purified by preparative HPLC (Method A) to afford 5-(4-(3-(2-(2,6-dioxopiperidin-3-yl)-1-oxoisoindolin-4-yl)prop-2-yn-1-yl)piperidin-1-yl)-N-(2-((R)-1-methylpyrrolidin-2-yl)-1H-pyrrolo[3,2-c]pyridin-6-yl)pyrazine-2-carboxamide (Example 154, 0.07 g, 29%) as off-white solid. ¹H NMR (400 MHz, DMSO-d₆) δ ppm 1.32-1.37 (m, 2H), 1.72-2.03 (m, 8H), 2.14-2.19 (m, 4H), 2.22-2.32 (m, 1H), 2.41-2.44 (m, 1H), 2.55-2.67 (m, 3H), 2.87-2.96 (m, 1H), 3.05-3.16 (m, 3H), 4.29-4.34 (m, 1H), 4.43-4.48 (m, 1H), 4.57-4.61 (m, 2H), 5.10-5.15 (m, 1H), 6.40 (s, 1H), 7.44 (d, J=10.0 Hz, 1H), 7.51 (d, J=7.6 Hz, 1H), 7.64 (d, J=7.4 Hz, 1H), 7.70 (d, J=7.4 Hz, 1H), 7.97 (d, J=9.6 Hz, 1H), 8.24 (s, 1H), 8.49 (s, 1H), 10.10 (br s, 1H), 11.01 (br s, 1H), 11.44 (br s, 1H). Mass spec m/z 686.0 [M+H]⁺.

### Example 155 was Synthesised Following Scheme 151

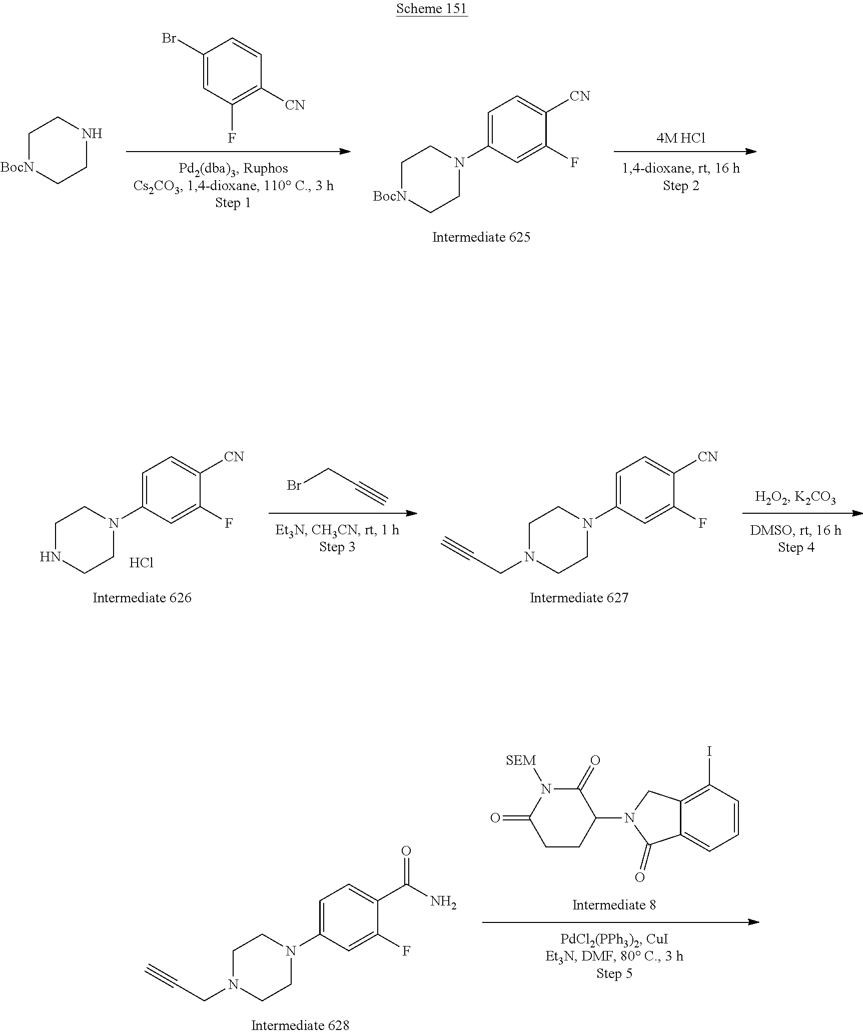

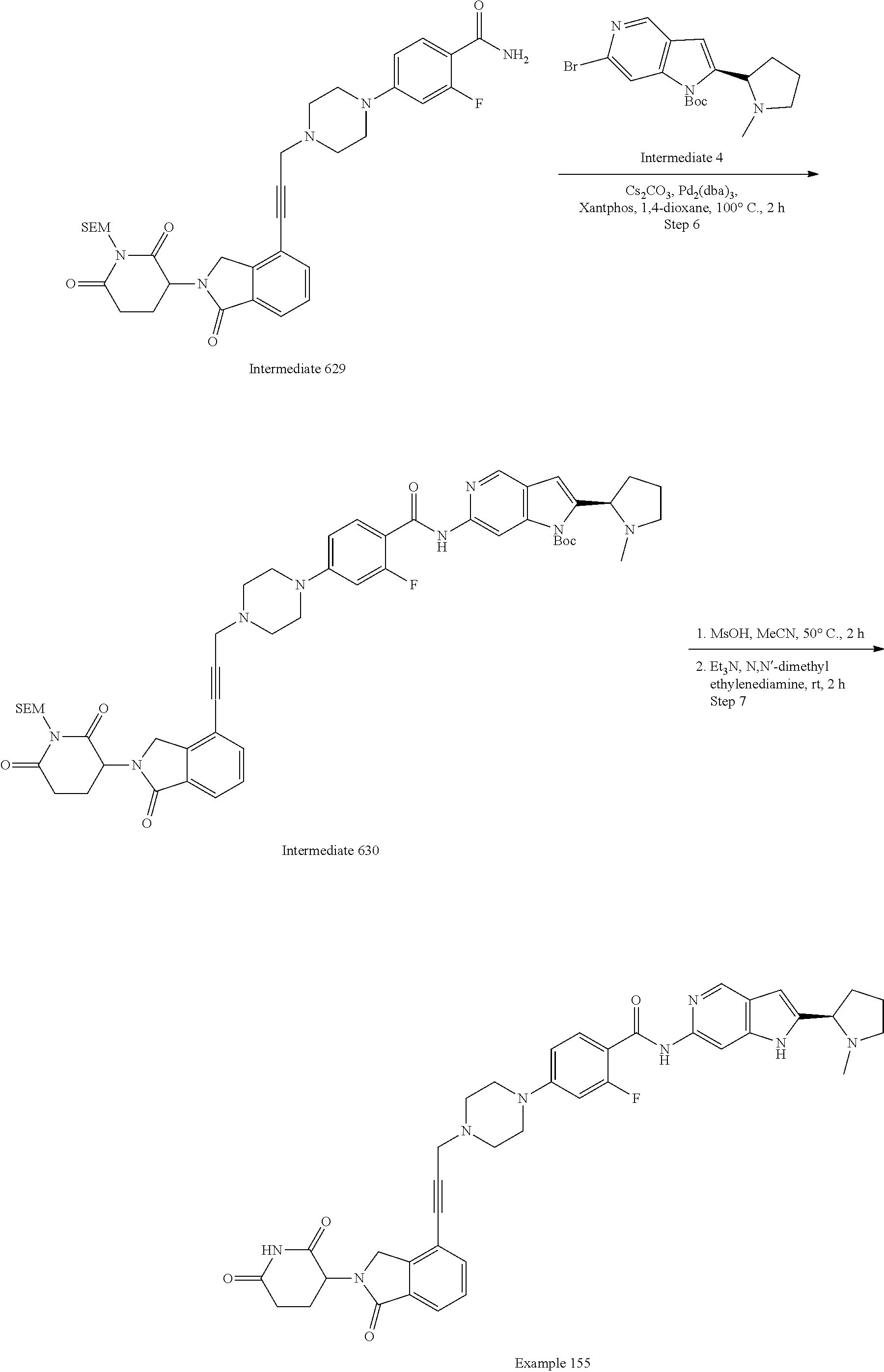

### Step 1

### Intermediate 625: tert-butyl 4-(4-cyano-3-fluorophenyl)piperazine-1-carboxylate

To stirred suspension of tert-butyl piperazine-1-carboxylate (CAS No: 57260-71-6, 2.8 g, 15 mmol) and 4-bromo-2-fluoro-benzonitrile (CAS No: 105942-08-3, 2.0 g, 10 mmol) in 1,4-dioxane (20 mL) was added cesium carbonate (5.8 g, 30 mmol). The reaction mixture was purged with argon for 15 min then Pd₂(dba)₃ (1.3 g, 1.5 mmol) and RuPhos (1.4 g, 3.0 mmol) were added. The reaction mixture was purged for another 10 min and heated at 110° C. for 3 h. After completion, the reaction mixture was concentrated in vacuo. The crude material was purified by combi-flash chromatography, eluting with 70% ethyl acetate in heptane, to afford tert-butyl 4-(4-cyano-3-fluorophenyl)piperazine-1-carboxylate (Intermediate 625, 1.50 g, 49%) as a yellow solid. ¹H NMR (400 MHz, DMSO-d₆) δ ppm 1.42 (s, 9H), 3.14 (s, 8H), 6.84 (d, J=9.2 Hz, 1H), 6.94 (d, J=14.0 Hz, 1H), 7.61 (t, J=8.4 Hz, 1H). Mass spec m/z 306.2 [M+H]⁺.

### Step 2

### Intermediate 626: 2-fluoro-4-(piperazin-1-yl)benzonitrile hydrochloride

To a cooled (0° C.) solution of tert-butyl 4-(4-cyano-3-fluoro-phenyl)piperazine-1-carboxylate (Intermediate 625, 2.5 g, 8.2 mmol) in dichloromethane (25 mL) was added 4M hydrochloric acid in 1,4-dioxane (41 mL, 160.0 mmol) and the reaction stirred at room temperature for 16 h. After completion, the reaction mixture was concentrated in vacuo. The crude material was triturated with diethyl ether (10 mL) and dried in vacuo to afford 2-fluoro-4-piperazin-1-yl-benzonitrile hydrochloride (Intermediate 626, 1.80 g, 91%) as an off-white solid, which was used in the next step without further purification. ¹H NMR (400 MHz, DMSO-d₆) δ ppm 3.17-3.19 (m, 4H), 3.64-3.67 (m, 4H), 6.93 (dd, J=8.8, 2.0 Hz, 1H), 7.06 (dd, J=14.0, 2.4 Hz, 1H), 7.68 (t, J=8.4 Hz, 1H), 9.4 (br s, 2H). Mass spec m/z 206.1 [M+H]⁺.

### Step 3

### Intermediate 627: 2-fluoro-4-(4-(prop-2-yn-1-yl)piperazin-1-yl)benzonitrile

To a cooled (0° C.) solution of 2-fluoro-4-piperazin-1-yl-benzonitrile hydrochloride (Intermediate 626, 1.8 g, 7.4 mmol) in acetonitrile (4 mL) were added triethylamine (5.2 mL, 37 mmol) and 3-bromoprop-1-yne (0.97 g, 8.2 mmol) and the reaction mixture was stirred at room temperature for 1 h. After completion, the reaction mixture was diluted with ice water (90 mL) and extracted with ethyl acetate (3×100 mL). The combined organic layers were dried over anhydrous Na₂SO₄, filtered and concentrated in vacuo. The crude material was purified by combi-flash chromatography, by eluting with 7% MeOH in DCM, to afford 2-fluoro-4-(4-(prop-2-yn-1-yl)piperazin-1-yl)benzonitrile (Intermediate 627, 1.2 g, 46%) as an off-white solid. ¹H NMR (400 MHz, DMSO-d₆) δ ppm 2.50-2.55 (m, 4H), 3.19-3.20 (m, 1H), 3.32-3.34 (m, 2H), 3.39-3.42 (m, 4H), 6.86 (dd, J=9.2, 2.4 Hz, 1H), 6.96 (dd, J=14.4, 2.4 Hz, 1H), 7.59 (t, J=8.4 Hz, 1H). Mass spec m/z 244.3 [M+H]⁺.

### Step 4

### Intermediate 628: 2-fluoro-4-(4-(prop-2-yn-1-yl)piperazin-1-yl)benzamide

To a cooled (0° C.) solution of 2-fluoro-4-(4-prop-2-ynylpiperazin-1-yl)benzonitrile (Intermediate 627, 0.60 g, 2.46 mmol) in dimethyl sulfoxide (6 mL) was added potassium carbonate (0.51 g, 3.69 mmol) and hydrogen peroxide (0.95 g, 9.86 mmol) and the reaction mixture was stirred at room temperature for 16 h. After completion, the reaction mixture was poured into ice water (50 mL), the resultant precipitate was collected by filtration and dried in vacuo to afford 2-fluoro-4-(4-prop-2-ynylpiperazin-1-yl)benzamide (Intermediate 628, 0.50 g, 78%) as a yellow liquid. ¹H NMR (400 MHz, DMSO-d₆) δ ppm 2.54-2.57 (m, 4H), 3.19-3.20 (m, 1H), 3.29-3.39 (m, 6H), 6.72-6.81 (m, 2H), 7.21 (s, 1H), 7.34 (s, 1H), 7.61 (t, J=8.4 Hz, 1H). Mass spec m/z 262.10 [M+H]⁺.

### Step 5

### Intermediate 629: 4-(4-(3-(2-(2,6-dioxo-1-((2-(trimethylsilyl)ethoxy)methyl)piperidin-3-yl)-1-oxoisoindolin-4-yl)prop-2-yn-1-yl)piperazin-1-yl)-2-fluorobenzamide

To a suspension of 2-fluoro-4-(4-prop-2-ynylpiperazin-1-yl)benzamide (Intermediate 628, 0.70 g, 2.68 mmol) and 3-(4-iodo-1-oxo-isoindolin-2-yl)-1-(2-trimethylsilylethoxymethyl)piperidine-2,6-dione (Intermediate 8, 1.07 g, 2.14 mmol) in acetonitrile (7 mL) was added N,N-diisopropylethylamine (1.75 g, 13.39 mmol). The reaction mixture was purged with argon for 15 min, then Pd(OAc)₂ acetate (0.06 g, 0.40 mmol), copper(I) iodide (0.08 g, 0.40 mmol) and triphenylphosphine (0.21 g, 0.80 mmol) were added. The reaction mixture was purged with argon for another 10 min and heated at 80° C. for 3 h. After completion, the reaction mixture was poured into ice cold water (70 mL) and extracted with ethyl acetate (2×120 mL). The combined organic layers were dried over anhydrous Na₂SO₄, filtered and concentrated in vacuo. The crude material was purified by combi-flash chromatography, by eluting with 7% MeOH in DCM, to afford 4-(4-(3-(2-(2,6-dioxo-1-((2-(trimethylsilyl)ethoxy)methyl)piperidin-3-yl)-1-oxoisoindolin-4-yl)prop-2-yn-1-yl)piperazin-1-yl)-2-fluorobenzamide (Intermediate 629, 1.0 g, 59%) as a dark liquid. ¹H NMR (400 MHz, DMSO-d₆) δ ppm −0.02 (s, 9H), 0.81-0.86 (m, 2H), 2.17-2.19 (m, 1H), 2.31-2.45 (m, 2H), 2.66-2.79 (m, 5H), 3.05-3.19 (m, 3H), 3.49-3.55 (m, 2H), 3.60-3.66 (m, 3H), 4.29-4.33 (m, 1H), 4.48-4.52 (m, 1H), 5.00-5.08 (m, 2H), 5.24-5.29 (m, 1H), 6.73-6.81 (m, 2H), 7.20 (s, 1H), 7.34 (s, 1H), 7.54-7.64 (m, 2H), 7.72 (d, J=7.6 Hz, 1H), 7.77 (d, J=7.2 Hz, 1H). Mass spec m/z 631.9 [M−H]⁻.

### Step 6

### Intermediate 630: tert-butyl 6-(4-(4-(3-(2-(2,6-dioxo-1-((2-(trimethylsilyl)ethoxy)methyl)piperidin-3-yl)-1-oxoisoindolin-4-yl)prop-2-yn-1-yl)piperazin-1-yl)-2-fluorobenzamido)-2-((R)-1-methylpyrrolidin-2-yl)-1H-pyrrolo[3,2-c]pyridine-1-carboxylate

To a suspension of 4-[4-[3-[2-[2,6-dioxo-1-(2-trimethylsilylethoxymethyl)-3-piperidyl]-1-oxo-isoindolin-4-yl]prop-2-ynyl]piperazin-1-yl]-2-fluoro-benzamide (Intermediate 629, 0.50 g, 0.79 mmol), tert-butyl 6-bromo-2-[(2R)-1-methylpyrrolidin-2-yl]pyrrolo[3,2-c]pyridine-1-carboxylate (Intermediate 4, 0.36 g, 0.95 mmol) in 1,4-dioxane (5 mL) was added cesium carbonate (0.30 g, 1.58 mmol). The resulting reaction mixture was purged with argon for 15 min, then Pd₂(dba)₃ (0.10 g, 0.11 mmol) and Xanthphos (0.07 g, 0.11 mmol) were added. The resulting reaction mixture degassed for another 10 min and stirred at 110° C. for 2 h. After completion of reaction, reaction mixture was concentrated in vacuo. The crude material was purified by combi-flash chromatography, by eluting with 7% MeOH in DCM, to afford tert-butyl 6-(4-(4-(3-(2-(2,6-dioxo-1-((2-(trimethylsilyl)ethoxy)methyl)piperidin-3-yl)-1-oxoisoindolin-4-yl)prop-2-yn-1-yl)piperazin-1-yl)-2-fluorobenzamido)-2-((R)-1-methylpyrrolidin-2-yl)-1H-pyrrolo[3,2-c]pyridine-1-carboxylate (Intermediate 630, 0.48 g, 65%) as a yellow solid. ¹H NMR (400 MHz, DMSO-d₆) δ ppm −0.1 (s, 9H), 0.80-0.86 (m, 1H), 1.16-1.18 (m, 12H), 2.05-2.10 (m, 1H), 2.32-2.45 (m, 3H), 2.68-2.79 (m, 4H), 3.02-3.12 (m, 2H), 3.38-3.40 (m, 5H), 3.50-3.61 (m, 6H), 3.65-3.70 (m, 2H), 3.89-3.93 (m, 1H), 4.29-4.33 (m, 1H), 4.48-4.52 (m, 1H), 5.00-5.08 (m, 2H), 5.24-5.29 (m, 1H), 6.74 (s, 1H), 6.82-6.88 (m, 2H), 7.54-7.58 (m, 1H), 7.69-7.78 (m, 3H), 8.55 (s, 1H), 8.93 (s, 1H), 9.85 (d, J=8.0 Hz, 1H). Mass spec m/z 932.7 [M]⁺.

### Step 7

### Example 155: 4-(4-(3-(2-(2,6-dioxopiperidin-3-yl)-1-oxoisoindolin-4-yl)prop-2-yn-1-yl)piperazin-1-yl)-2-fluoro-N-(2-((R)-1-methylpyrrolidin-2-yl)-1H-pyrrolo[3,2-c]pyridin-6-yl)benzamide

To a cooled (0° C.) solution of 6-(4-(4-(3-(2-(2,6-dioxo-1-((2-(trimethylsilyl)ethoxy)methyl)piperidin-3-yl)-1-oxoisoindolin-4-yl)prop-2-yn-1-yl)piperazin-1-yl)-2-fluorobenzamido)-2-((R)-1-methylpyrrolidin-2-yl)-1H-pyrrolo[3,2-c]pyridine-1-carboxylate (Intermediate 630, 0.43 g, 0.42 mmol) in acetonitrile (4 mL) was added methanesulfonic acid (0.43 g, 4.29 mmol) and the reaction mixture was heated at 50° C. for 2 h. The reaction mixture was cooled to room temperature, then N,N′-dimethylethylenediamine (0.24 g, 2.57 mmol) and triethylamine (0.91 g, 8.57 mmol) were added and the reaction mixture was stirred at room temperature for 2 h. After completion, the reaction mixture was concentrated in vacuo. The residue was diluted with ice cold water (100 mL) and stirred for 5 min. The resultant precipitate was collected by filtration and dried in vacuo. The crude material was purified by preparative HPLC (Method A) to afford 4-(4-(3-(2-(2,6-dioxopiperidin-3-yl)-1-oxoisoindolin-4-yl)prop-2-yn-1-yl)piperazin-1-yl)-2-fluoro-N-(2-((R)-1-methylpyrrolidin-2-yl)-1H-pyrrolo[3,2-c]pyridin-6-yl)benzamide (Example 155, 0.06 g, 14%) as a white solid. ¹H NMR (400 MHz, DMSO-d₆) δ ppm 1.78-1.94 (m, 3H), 1.98-2.04 (m, 1H), 2.12-2.18 (m, 4H), 2.22-2.29 (m, 1H), 2.40-2.49 (m, 1H), 2.53-2.60 (m, 2H), 2.62-2.71 (m, 4H), 2.83-2.92 (m, 1H), 3.11-3.17 (m, 1H), 3.38-3.41 (m, 4H), 3.68-3.70 (m, 2H), 4.32-4.36 (m, 1H), 4.46-4.50 (m, 1H), 5.10-5.15 (m, 1H), 6.38 (s, 1H), 6.82-6.89 (m, 2H), 7.55 (t, J=7.6 Hz, 1H), 7.69-7.76 (m, 3H), 8.20 (s, 1H), 8.44 (s, 1H), 9.55 (d, J=8.8 Hz, 1H), 11.01 (s, 1H), 11.38 (s, 1H). Mass spec m/z 703.3 [M−H]⁻.

### Example 156 was Synthesised Following Scheme 152

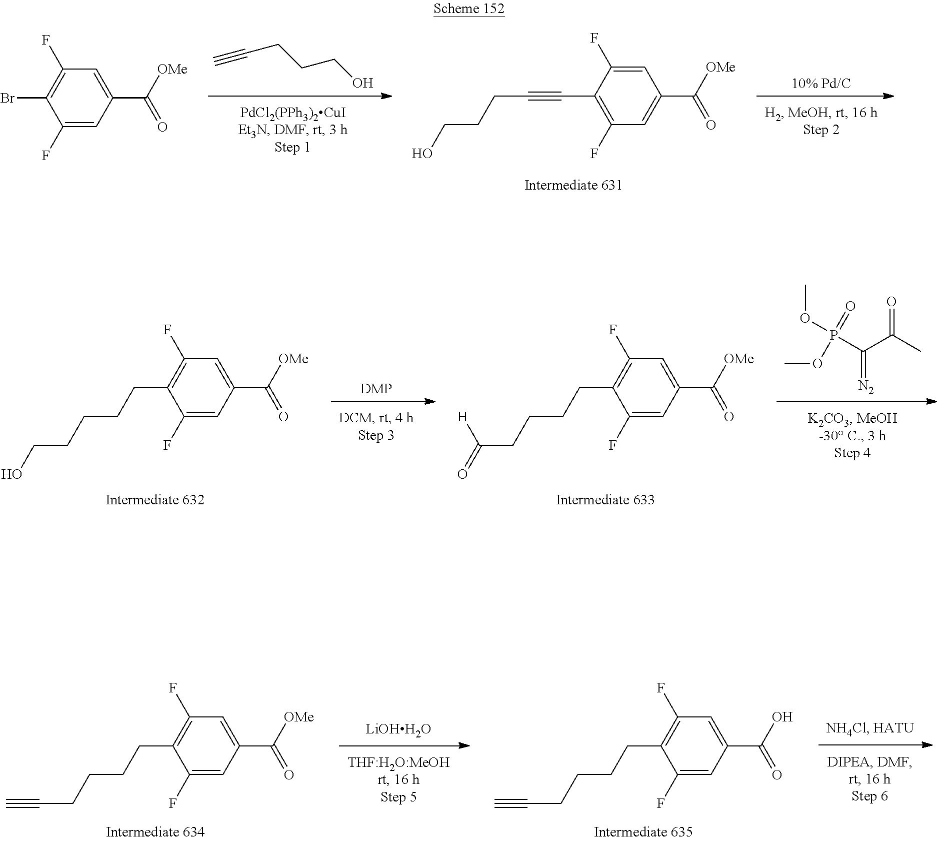

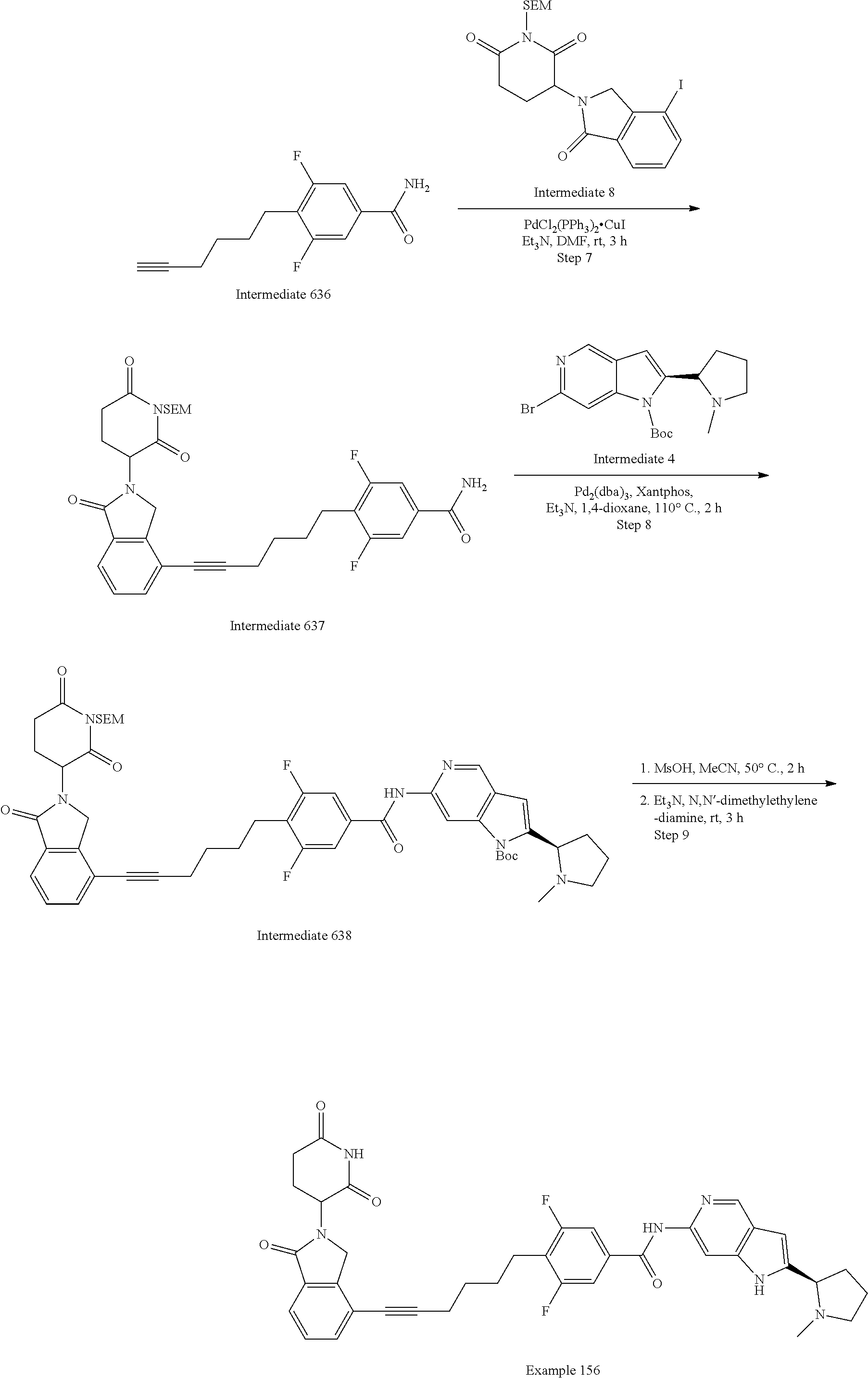

### Step 1

### Intermediate 631: methyl 3,5-difluoro-4-(5-hydroxypent-1-yn-1-yl)benzoate

To a solution of methyl-4-bromo-3,5-difluorobenzoate (CAS No. 1803565-64-18.8 g, 35.0 mmol) and pent-4-yn-1-ol (5.3 g, 63.0 mmol) in dimethylformamide (50 mL) was added triethylamine (180 mL, 1300.0 mmol). The reaction mixture was purged with argon for 15 min, then PdCl₂(PPh₃)₂ (2.5 g, 3.5 mmol) and copper(I) iodide (0.68 g, 3.5 mmol) were added. The reaction mixture was purged with argon for another 10 min and stirred at room temperature for 3 h. After completion, the reaction mixture was concentrated in vacuo, diluted with water (300 mL) and extracted with ethyl acetate (3×200 mL). The combined organic layers were dried over anhydrous Na₂SO₄ and concentrated in vacuo. The crude material was purified by combi-flash chromatography, by eluting with 90% ethyl acetate in heptane, to afford methyl 3,5-difluoro-4-(5-hydroxypent-1-yn-1-yl)benzoate (Intermediate 631, 7.6 g, 85%) as a yellow liquid. ¹H NMR (400 MHz, DMSO-d₆) δ ppm 1.69-1.73 (m, 2H), 2.58-2.62 (m, 2H), 3.50-3.55 (m, 2H), 3.88 (s, 3H), 4.56-4.59 (m, 1H), 7.65 (d, J=7.6 Hz, 2H). Mass spec m/z 255.13 [M+H]⁺.

### Step 2

### Intermediate 632: methyl 3,5-difluoro-4-(5-hydroxypentyl)benzoate

To a solution of methyl 3,5-difluoro-4-(5-hydroxypent-1-ynyl)benzoate (Intermediate 631, 7.7 g, 30 mmol) in methanol (80 mL), was added 10% Pd/C (3.0 g) and the reaction mixture was stirred at room temperature for 16 h under hydrogen atmosphere at 100 psi. The reaction mixture was then filtered through a celite bed, the filter pad was washed with ethyl acetate (100 mL) and the filtrate concentrated in vacuo to afford methyl 3,5-difluoro-4-(5-hydroxypentyl)benzoate (Intermediate 632, 7.0 g, 89%) as a colourless liquid, which was directly used in next step without further purification. ¹H NMR (400 MHz, DMSO-d₆) δ ppm 1.26-1.34 (m, 2H), 1.34-1.45 (m, 2H), 1.49-1.56 (m, 2H), 2.63-2.67 (m, 2H), 3.33-3.38 (m, 2H), 3.86 (s, 3H), 4.32-4.35 (m, 1H), 7.53 (d, J=7.6 Hz, 2H).

### Step 3

### Intermediate 633: methyl 3,5-difluoro-4-(5-oxopentyl)benzoate

To a cooled (0° C.) solution of methyl 3,5-difluoro-4-(5-hydroxypentyl)benzoate (Intermediate 632, 6 g, 23.23 mmol) in dichloromethane (100 mL) was added Dess-Martin periodinane (15.24 g, 34.84 mmol) and the reaction mixture was stirred at room temperature for 4 h. After completion, the reaction mixture was diluted with saturated aqueous sodium bicarbonate solution (300 mL) and saturated aqueous sodium thiosulphate solution (300 mL), then extracted with dichloromethane (3×200 mL). The combined organic layers were dried over anhydrous Na₂SO₄ and concentrated in vacuo to afford methyl 3,5-difluoro-4-(5-oxopentyl)benzoate (Intermediate 633, 4.9 g, 82%) as a yellow liquid, which was used for next step without further purification. ¹H NMR (400 MHz, DMSO-d₆) δ ppm 1.49-1.50 (m, 2H), 2.21-2.25 (m, 2H), 2.46-2.50 (m, 2H), 2.65-2.68 (m, 2H), 3.86 (s, 3H), 7.55 (d, J=6.8 Hz, 2H), 9.64 (s, 1H).

### Step 4

### Intermediate 634: methyl 3,5-difluoro-4-(hex-5-yn-1-yl)benzoate

To a cooled (−30° C.) solution of methyl 3,5-difluoro-4-(5-oxopentyl)benzoate (Intermediate 633, 4.9 g, 19 mmol) in methanol (100 mL) was added potassium carbonate (3.2 g, 23 mmol) and 1-diazo-1-dimethoxyphosphoryl-propan-2-one (5.5 g, 29 mmol) and the resulting reaction mixture was stirred at −30° C. for 3 h. After completion, the reaction mixture was concentrated in vacuo, diluted with water (300 mL) and extracted with diethyl ether (3×300 mL). The combined organic layers were dried over anhydrous Na₂SO₄ and concentrated in vacuo to obtain crude product. The crude product was purified by combi-flash chromatography, by eluting with 10% ethyl acetate in heptane, to afford methyl 3,5-difluoro-4-(hex-5-yn-1-yl)benzoate (Intermediate 634, 2.5 g, 52%) as a yellow liquid. ¹H NMR (400 MHz, DMSO-d₆) δ ppm 1.44-1.49 (m, 2H), 1.59-1.67 (m, 2H), 2.16-2.21 (m, 2H), 2.67-2.71 (m, 2H), 2.74-2.75 (m, 1H), 3.85 (s, 3H), 7.56 (d, J=7.2 Hz, 2H).

### Step 5

### Intermediate 635: 3,5-difluoro-4-(hex-5-yn-1-yl)benzoic acid

To solution of methyl 3,5-difluoro-4-(hex-5-yn-1-yl)benzoate (Intermediate 634, 2.50 g, 9.91 mmol) in tetrahydrofuran (15 mL), methanol (15 mL) and water (7 mL) was added lithium hydroxide (0.96 g, 39.6 mmol) and the reaction mixture was stirred at room temperature for 16 h. After completion, the reaction mixture was concentrated in vacuo. The obtained residue was acidified to pH~4 with 1N aqueous HCl solution, the resultant precipitate was collected by filtration, washed with water (20 mL) and heptane (20 mL), then dried in vacuo to afford 3,5-difluoro-4-(hex-5-yn-1-yl)benzoic acid (Intermediate 635, 2.0 g, 85%) as an off-white solid, which was used for next step without further purification. ¹H NMR (400 MHz, DMSO-d₆) δ ppm 1.45-1.49 (m, 2H), 1.60-1.66 (m, 2H), 2.15-2.20 (m, 2H), 2.63-2.70 (m, 2H), 2.74-2.75 (m, 1H), 7.54 (d, J=7.2 Hz, 2H), 13.13-1.16 (bs, 1H). Mass spec m/z 237.3 [M−H]⁻.

### Step 6

### Intermediate 636: 3,5-difluoro-4-(hex-5-yn-1-yl)benzamide

To a stirred solution of 3,5-difluoro-4-hex-5-ynyl-benzoic acid (Intermediate 635, 1.5 g, 6.3 mmol) in dimethylformamide (15 mL) was added HATU (3.8 g, 9.4 mmol) and the reaction mixture was stirred at room temperature for 15 min. Ammonium chloride (1.7 g, 31 mmol) and N,N-diisopropylethylamine (2.5 g, 19 mmol) were then added and the reaction mixture was stirred at room temperature for 16 h. After completion, the reaction mixture was quenched with ice cold water (200 mL), the resultant precipitate was collected by filtration and dried in vacuo to afford 3,5-difluoro-4-(hex-5-yn-1-yl)benzamide (Intermediate 636, 1.3 g, 87%) as a white solid. ¹H NMR (400 MHz, DMSO-d₆) δ ppm 1.43-1.48 (m, 2H), 1.59-1.63 (m, 2H), 2.17-2.19 (m, 2H), 2.65-2.67 (m, 2H), 2.74-2.76 (m, 1H), 7.53-7.62 (m, 3H), 8.07 (s, 1H). Mass spec m/z 236.3[M−H]⁻.

### Step 7

### Intermediate 637: 4-(6-(2-(2,6-dioxo-1-((2-(trimethylsilyl)ethoxy)methyl)piperidin-3-yl)-1-oxoisoindolin-4-yl)hex-5-yn-1-yl)-3,5-difluorobenzamide

To a solution of 3-(4-iodo-1-oxoisoindolin-2-yl)-1-((2-(trimethylsilyl)ethoxy)methyl)piperidine-2,6-dione (Intermediate 8, 1.055 g, 2.11 mmol) and 3,5-difluoro-4-(hex-5-yn-1-yl)benzamide (Intermediate 636, 0.5 g, 2.11 mmol) in dimethylformamide (10 mL) was added triethylamine (7.72 g, 75.89 mmol). The reaction mixture was purged with argon for 15 min, then PdCl₂(PPh₃)₂ (0.15 g, 0.21 mmol) and copper(I) iodide (0.041 g, 0.21 mmol) were added. The reaction mixture was purged with argon for another 10 min and stirred at room temperature for 3 h. After completion, the reaction mixture was diluted with water (200 mL) and extracted with ethyl acetate (3×90 mL). The combined organic layers were dried over anhydrous Na₂SO₄ and concentrated in vacuo. The crude material was purified by combi-flash chromatography, by eluting with 80% ethyl acetate in heptane, to afford 4-(6-(2-(2,6-dioxo-1-((2-(trimethylsilyl)ethoxy)methyl)piperidin-3-yl)-1-oxoisoindolin-4-yl)hex-5-yn-1-yl)-3,5-difluorobenzamide (Intermediate 637, 0.7 g, 54.5%) as a yellow solid. ¹H NMR (400 MHz, DMSO-d₆) δ ppm −0.041 (s, 9H), 0.79-0.84 (m, 2H), 1.55-1.64 (m, 2H), 1.66-1.64 (m, 2H), 2.04-2.11 (m, 1H), 2.30-2.43 (m, 2H), 2.63-2.72 (m, 4H), 2.73-2.77 (m, 1H), 3.50-3.58 (m, 2H), 4.22-4.26 (m, 1H), 4.42-4.46 (m, 1H), 5.01-5.09 (m, 2H), 5.24-5.29 (m, 1H), 7.51-7.63 (m, 4H), 7.72 (d, J=7.6 Hz, 1H), 7.95 (s, 1H), 8.07 (s, 1H). Mass spec m/z 608.0 [M−H]⁻.

### Step 8

### Intermediate 638: tert-butyl 6-(4-(6-(2-(2,6-dioxo-1-((2-(trimethylsilyl)ethoxy)methyl)piperidin-3-yl)-1-oxoisoindolin-4-yl)hex-5-yn-1-yl)-3,5-difluorobenzamido)-2-((R)-1-methylpyrrolidin-2-yl)-1H-pyrrolo[3,2-c]pyridine-1-carboxylate

To a solution of 4-(6-(2-(2,6-dioxo-1-((2-(trimethylsilyl)ethoxy)methyl)piperidin-3-yl)-1-oxoisoindolin-4-yl)hex-5-yn-1-yl)-3,5-difluorobenzamide (Intermediate 637, 0.64 g, 1.05 mmol) in 1,4-dioxane (15 mL) were added tert-butyl (R)-6-bromo-2-(1-methylpyrrolidin-2-yl)-1H-pyrrolo[3,2-c]pyridine-1-carboxylate (Intermediate 4, 0.5 g, 1.31 mmol) and cesium carbonate (1.28 g, 3.94 mmol). The reaction mixture was purged with argon for 15 min, then Xantphos (0.22 g, 0.39 mmol) and Pd₂(dba)₃ (0.186 g, 0.20 mmol) were added. The resulting reaction was further purged with argon for 10 min and stirred for 100° C. for 2 h. After completion, the reaction mixture was filtered through a celite bed, the filter pad was washed with ethyl acetate (100 mL), and the filtrate was concentrated in vacuo. The crude material was purified by combi-flash chromatography, by eluting with 90% ethyl acetate in heptane, to afford tert-butyl 6-(4-(6-(2-(2,6-dioxo-1-((2-(trimethylsilyl)ethoxy)methyl)piperidin-3-yl)-1-oxoisoindolin-4-yl)hex-5-yn-1-yl)-3,5-difluorobenzamido)-2-((R)-1-methylpyrrolidin-2-yl)-1H-pyrrolo[3,2-c]pyridine-1-carboxylate (Intermediate 638, 0.40 g, 33%) as a yellow solid. Mass spec m/z 908.6 [M]⁺.

### Step 9

### Example 156: 4-(6-(2-(2,6-dioxopiperidin-3-yl)-1-oxoisoindolin-4-yl)hex-5-yn-1-yl)-3,5-difluoro-N-(2-((R)-1-methylpyrrolidin-2-yl)-1H-pyrrolo[3,2-c]pyridin-6-yl)benzamide

To a stirred solution of tert-butyl 6-(4-(6-(2-(2,6-dioxo-1-((2-(trimethylsilyl)ethoxy)methyl)piperidin-3-yl)-1-oxoisoindolin-4-yl)hex-5-yn-1-yl)-3,5-difluorobenzamido)-2-((R)-1-methylpyrrolidin-2-yl)-1H-pyrrolo[3,2-c]pyridine-1-carboxylate (Intermediate 638, 0.4 g, 0.44 mmol) in acetonitrile (15 mL) was added methanesulfonic acid (0.29 mL, 4.40 mmol) and the reaction was heated at 50° C. for 2 h. The reaction was cooled to room temperature, N,N′-dimethylethylenediamine (0.21 g, 2.20 mmol) and triethylamine (1.23 mL, 8.80 mmol) were added and the reaction stirred at room temperature for 3 h. After completion, the reaction mixture was diluted with water (50 mL), resulting in a solid precipitate, which was collected by filtration and dried in vacuo. The crude material was purified by preparative HPLC (Method A) to afford 4-(6-(2-(2,6-dioxopiperidin-3-yl)-1-oxoisoindolin-4-yl)hex-5-yn-1-yl)-3,5-difluoro-N-(2-((R)-1-methylpyrrolidin-2-yl)-1H-pyrrolo[3,2-c]pyridin-6-yl)benzamide (Example 156, 0.042 g, 14%) as a white solid. ¹H NMR (400 MHz, DMSO-d₆) δ ppm 1.59-1.68 (m, 2H), 1.71-1.95 (m, 5H), 1.99-2.17 (m, 2H), 2.13-2.20 (m, 4H), 2.23-2.31 (m, 1H), 2.41-2.46 (m, 1H), 2.53-2.57 (m, 3H), 2.73-2.79 (m, 2H), 2.88-2.98 (m, 1H), 3.12-3.19 (m, 1H), 3.33-3.39 (m, 1H), 4.27-4.46 (m, 2H), 5.12-5.17 (m, 1H), 6.40 (s, 1H), 7.52 (t, J=7.6 Hz, 1H), 7.70 (d, J=7.2 Hz, 1H), 7.77 (d, J=8.0 Hz, 2H), 8.17 (s, 1H), 8.51 (s, 1H), 10.64 (br s, 1H), 11.00 (br s, 1H), 11.39 (br s, 1H). Mass spec m/z 679.1[M+H]⁺.

### Example 157 was Synthesised Following Scheme 153

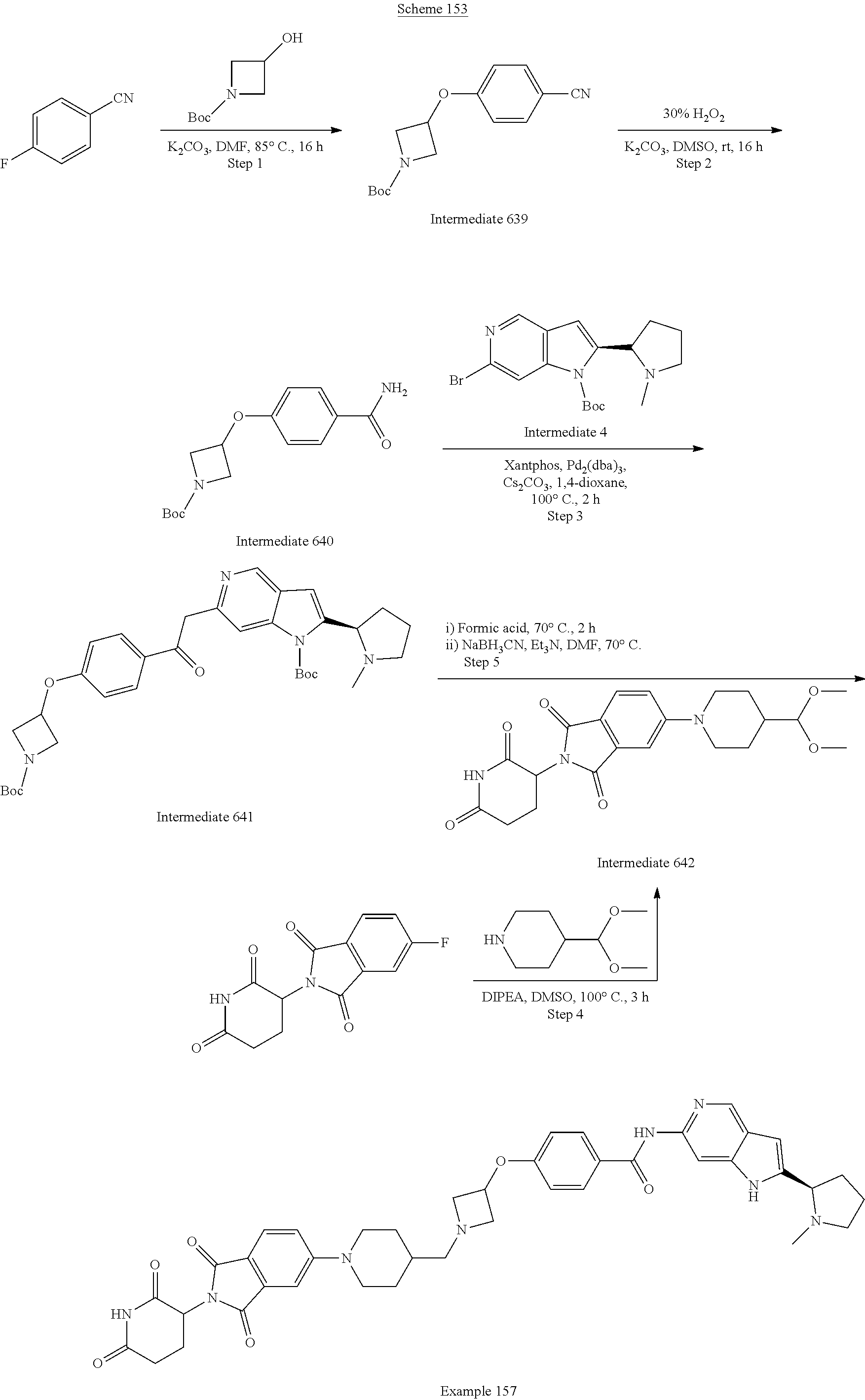

### Step 1

### Intermediate 639: tert-butyl 3-(4-cyanophenoxy)azetidine-1-carboxylate

To a solution of tert-butyl 3-hydroxyazetidine-1-carboxylate (CAS No. 141699-55-0, 2.86 g, 16.5 mmol) in dimethylformamide (40 mL) was added potassium carbonate (6.83 g, 49.5 mmol) followed by 4-fluorobenzonitrile (CAS No. 1194-02-1, 2.0 g, 16.5 mmol) and the reaction mixture was heated at 85° C. for 16 h. The reaction mixture was then diluted with water (300 mL) and extracted with ethyl acetate (3×100 mL). The combined organic layers were dried over anhydrous Na₂SO₄, filtered, and concentrated in vacuo. The crude material was purified by combi-flash chromatography, by eluting with 90% ethyl acetate in heptane, to afford tert-butyl 3-(4-cyanophenoxy)azetidine-1-carboxylate (Intermediate 639, 3.6 g, 79%) as a white solid. ¹H NMR (400 MHz, DMSO-d₆) δ ppm 1.36 (s, 9H), 3.75-3.83 (m, 2H), 4.28-4.35 (m, 2H), 5.05-5.10 (m, 1H), 7.00 (d, J=8.77 Hz, 2H), 7.77 (d, J=8.77 Hz, 2H).

### Step 2

### Intermediate 640: tert-butyl 3-(4-carbamoylphenoxy)azetidine-1-carboxylate

To a cooled (0° C.) solution of tert-butyl 3-(4-cyanophenoxy)azetidine-1-carboxylate (Intermediate 639, 3.60 g, 13.0 mmol) in dimethyl sulfoxide (15 mL) was added potassium carbonate (3.60 g, 26.0 mmol) and 30% hydrogen peroxide (3.10 mL, 39.0 mmol) dropwise. The reaction mixture was stirred at room temperature for 16 h then quenched with ice cold water (150 mL). The resultant precipitate was collected by filtration and dried in vacuo to afford tert-butyl 3-(4-carbamoylphenoxy)azetidine-1-carboxylate (Intermediate 640, 3.3 g, 86%) as off-white solid, which was used for the next step without further purification. ¹H NMR (400 MHz, DMSO-d₆) δ ppm 1.38 (s, 9H), 3.75-3.83 (m, 2H), 4.28-4.36 (m, 2H), 5.01-5.08 (m, 1H), 6.87 (d, J=7.89 Hz, 2H), 7.22 (br s, 1H), 7.81-7.84 (m, 3H). Mass spec m/z 291.0 [M−H]⁻

### Step 3

### Intermediate 641: tert-butyl (R)-6-(4-((1-(tert-butoxycarbonyl)azetidin-3-yl)oxy)benzamido)-2-(1-methylpyrrolidin-2-yl)-1H-pyrrolo[3,2-c]pyridine-1-carboxylate

To a solution of tert-butyl 3-(4-carbamoylphenoxy)azetidine-1-carboxylate (Intermediate 640, 0.50 g, 1.71 mmol) in 1,4-dioxane (20 mL) was added tert-butyl (R)-6-bromo-2-(1-methylpyrrolidin-2-yl)-1H-pyrrolo[3,2-c]pyridine-1-carboxylate (Intermediate 4, 0.78 g, 2.05 mmol) followed by cesium carbonate (1.67 g, 5.13 mmol). The reaction mixture was purged with argon for 15 min, then Pd₂(dba)₃ (0.24 g, 0.25 mmol) and Xantphos (0.30 g, 0.51 mmol) were added. The reaction mixture was further purged with argon for 10 min and heated at 100° C. for 2 h. The reaction mixture was filtered through a celite bed and the filter pad was washed with ethyl acetate (300 mL). The filtrate was concentrated in vacuo and the crude material was purified by combi-flash chromatography, by eluting with 90% ethyl acetate in heptane, to afford tert-butyl (R)-6-(4-((1-(tert-butoxycarbonyl)azetidin-3-yl)oxy)benzamido)-2-(1-methylpyrrolidin-2-yl)-1H-pyrrolo[3,2-c]pyridine-1-carboxylate (Intermediate 641, 0.8 g, 79%) as a yellow solid. ¹H NMR (400 MHz, DMSO-d₆) δ ppm 1.37 (s, 9H), 1.55-1.80 (m, 11H), 2.26-2.32 (m, 5H), 2.50-2.54 (m, 1H), 3.07-3.15 (m, 1H), 3.76-3.84 (m, 2H), 3.86-3.93 (m, 1H), 4.28-4.37 (m, 2H), 5.04-5.11 (m, 1H), 6.72 (s, 1H), 6.87-6.96 (m, 2H), 8.03 (d, J=8.33 Hz, 2H), 8.56 (s, 1H), 8.89 (s, 1H), 10.58 (br s, 1H). Mass spec m/z 592.4 [M+H]⁺.

### Step 4

### Intermediate 642: 5-(4-(dimethoxymethyl)piperidin-1-yl)-2-(2,6-dioxopiperidin-3-yl)isoindoline-1,3-dione

A solution of 2-(2,6-dioxopiperidin-3-yl)-5-fluoroisoindoline-1,3-dione (CAS No. 835616-61-0, 3.0 g, 11.0 mmol) in dimethyl sulfoxide (30 mL) was stirred at room temperature for 5 min. 4-(Dimethoxymethyl)piperidine (CAS No. 188646-83-5, 1.70 g, 11.0 mmol) followed by N,N-diisopropylethylamine (5.7 mL, 33.0 mmol) were the added at 0° C. and the reaction mixture was heated at 100° C. for 3 h. The reaction mixture was then diluted with water (15 mL) and extracted with ethyl acetate (25 mL). The combined organic layers were dried over anhydrous Na₂SO₄, filtered, and concentrated in vacuo. The crude material was purified by combi-flash chromatography, by eluting with 80% ethyl acetate in heptane, to afford 5-(4-(dimethoxymethyl)piperidin-1-yl)-2-(2,6-dioxopiperidin-3-yl)isoindoline-1,3-dione (Intermediate 642, 2.95 g, 65%) as a yellow solid. ¹H NMR (400 MHz, DMSO-d₆) δ ppm 1.19-1.32 (m, 2H), 1.65-1.73 (m, 2H), 1.86-2.04 (m, 2H), 2.52-2.60 (m, 2H), 2.80-2.96 (m, 3H), 3.24 (s, 6H), 3.99-4.09 (m, 3H), 5.01-5.08 (m, 1H), 7.21 (d, J=8.77 Hz, 1H), 7.28 (s, 1H), 7.63 (d, J=8.33 Hz, 1H), 11.06 (br s, 1H). Mass spec m/z 416.2 [M+H]⁺.

### Step 5

### Example 157: 4-((1-((1-(2-(2,6-dioxopiperidin-3-yl)-1,3-dioxoisoindolin-5-yl)piperidin-4-yl)methyl)azetidin-3-yl)oxy)-N-(2-((R)-1-methylpyrrolidin-2-yl)-1H-pyrrolo[3,2-c]pyridin-6-yl)benzamide

A solution of tert-butyl (R)-6-(4-((1-(tert-butoxycarbonyl)azetidin-3-yl)oxy)benzamido)-2-(1-methylpyrrolidin-2-yl)-1H-pyrrolo[3,2-c]pyridine-1-carboxylate (Intermediate 641, 0.50 g, 0.84 mmol) and 5-(4-(dimethoxymethyl)piperidin-1-yl)-2-(2,6-dioxopiperidin-3-yl)isoindoline-1,3-dione (Intermediate 642, 0.35 g, 0.84 mmol) in formic acid (10 mL) was heated at 70° C. for 2 h. The reaction mixture was concentrated in vacuo. Then dimethylformamide (10 mL) and triethylamine (0.59 mL, 4.22 mmol) were added, and the reaction mixture was stirred at room temperature for 15 min. Sodium cyanoborohydride (0.24 g 3.38 mmol) was then added and the reaction mixture was heated at 70° C. for 4 h. The reaction mixture was then quenched with water (50 mL), the resultant precipitate was collected by filtration and dried in vacuo. The crude material was purified by preparative HPLC (Method B) to afford 4-((1-((1-(2-(2,6-dioxopiperidin-3-yl)-1,3-dioxoisoindolin-5-yl)piperidin-4-yl)methyl)azetidin-3-yl)oxy)-N-(2—((R)-1-methylpyrrolidin-2-yl)-1H-pyrrolo[3,2-c]pyridin-6-yl)benzamide (Example 157, 0.052 g, 8%) as an off-white solid. ¹H NMR (400 MHz, DMSO-d₆) δ ppm 1.16-1.21 (m, 2H), 1.56-1.67 (m, 1H), 1.75-1.80 (m, 2H), 1.82-1.95 (m, 2H), 1.97-2.05 (m, 1H), 2.09-2.15 (m, 1H), 2.16 (s, 3H), 2.22-2.30 (m, 1H), 2.36-2.38 (m, 2H), 2.56-2.58 (m, 1H), 2.83-2.90 (m, 1H), 2.91-2.97 (m, 2H), 2.99-3.04 (m, 2H), 3.13-3.17 (m, 1H), 3.28-3.31 (m, 2H), 3.76-3.81 (m, 2H), 4.00-4.04 (m, 2H), 4.87-4.90 (m, 1H), 5.03-5.06 (m, 1H), 6.38 (s, 1H), 6.92 (d, J=8.88 Hz, 2H), 7.22 (dd, J=8.69, 2.19 Hz, 1H), 7.30 (s, 1H), 7.64 (d, J=8.50 Hz, 1H), 8.02 (d, J=8.88 Hz, 2H), 8.15-8.18 (m, 2H), 8.49 (s, 1H), 10.30 (br s, 1H), 11.07 (br s, 1H), 11.35 (br s, 1H). Mass spec m/z 745.3 [M+H]⁺.

### Example 158 was Synthesised Following Scheme 154

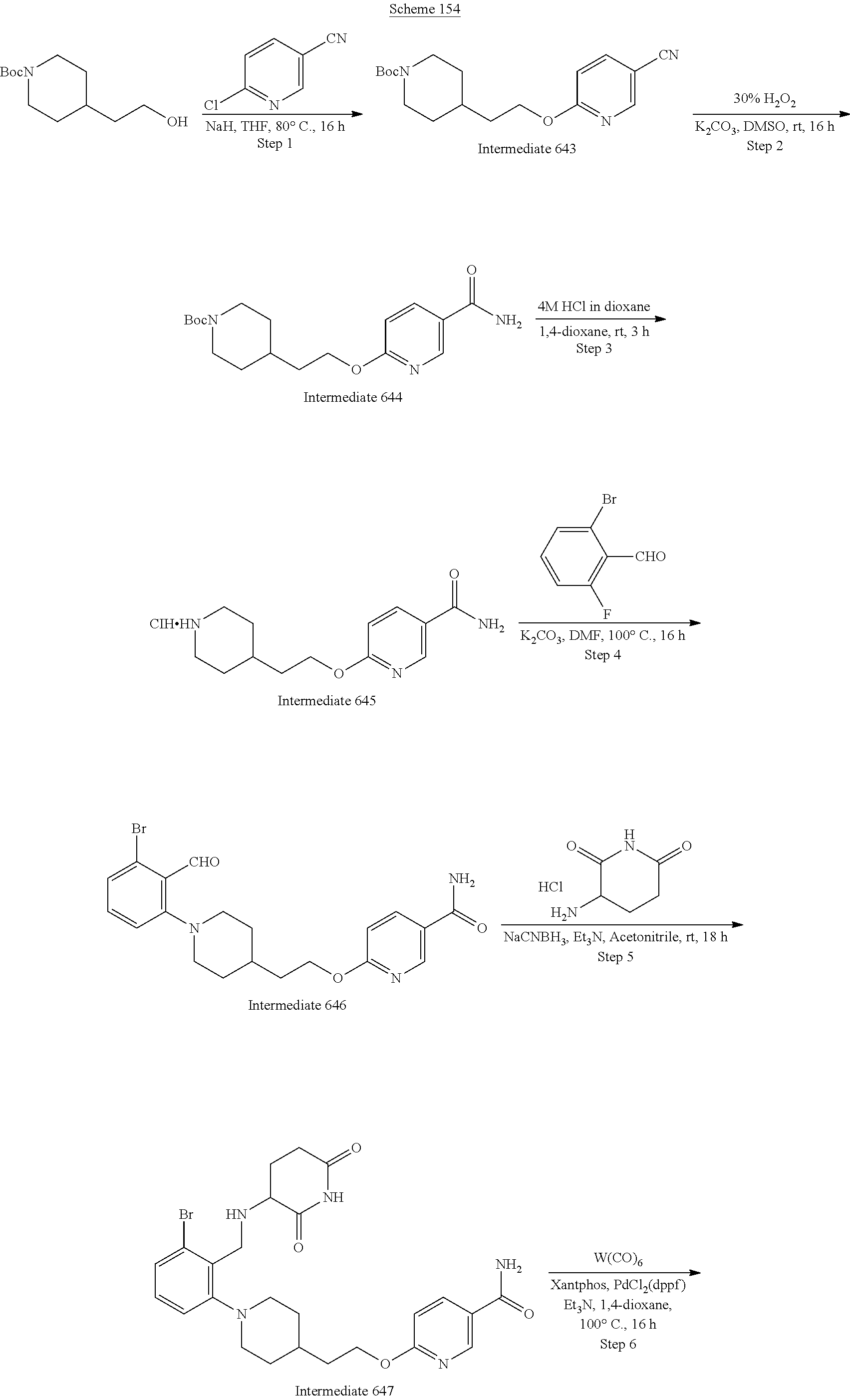

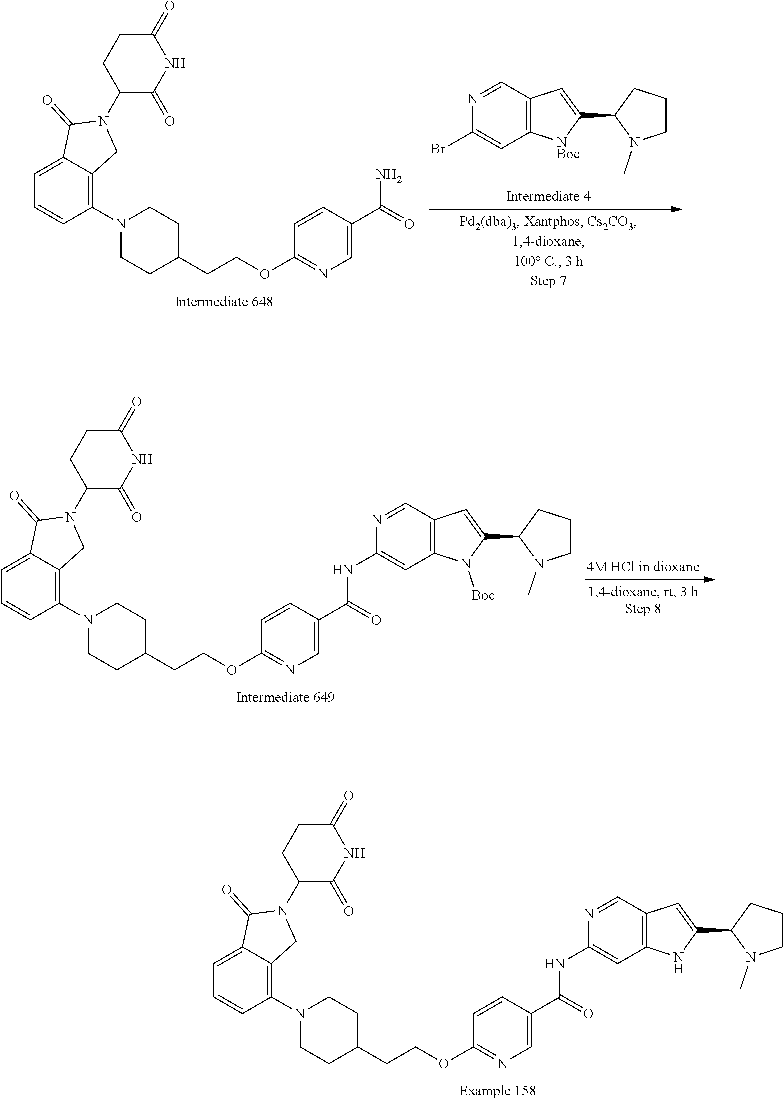 [SMILES: NC(=O)c1ccc(OCCC2CCN(c3cccc4c3CN(C3CCC(=O)NC3=O)C4=O)CC2)nc1 ; CN1CCC[C@@H]1c1cc2cnc(Br)cc2n1C(=O)OC(C)(C)C ; CN1CCC[C@@H]1c1cc2cnc(NC(=O)c3ccc(OCCC4CCN(c5cccc6c5CN(C5CCC(=O)NC5=O)C6=O)CC4)nc3)cc2n1C(=O)OC(C)(C)C ; CN1CCC[C@@H]1c1cc2cnc(NC(=O)c3ccc(OCCC4CCN(c5cccc6c5CN(C5CCC(=O)NC5=O)C6=O)CC4)nc3)cc2[nH]1]

### Step 1

### Intermediate 643: tert-butyl 4-(2-((5-cyanopyridin-2-yl)oxy)ethyl)piperidine-1-carboxylate

To a cooled (0° C.) solution of tert-butyl 4-(2-hydroxyethyl)piperidine-1-carboxylate (CAS No. 89151-44-0, 10.0 g, 41.4 mmol) in tetrahydrofuran (50 mL) was added sodium hydride (1.65 g, 62.1 mmol) and the reaction mixture was stirred for 30 m at 0)C prior to the addition of 6-chloronicotonitrile (CAS No. 33252-28-7, 5.85 g, 41.4 mmol). The reaction mixture was then heated to 80° C. for 16 h, then diluted with water (600 mL) and extracted with ethyl acetate (3×500 mL). The combined organic layers were dried over anhydrous Na₂SO₄, filtered and concentrated in vacuo to afford tert-butyl 4-(2-((5-cyanopyridin-2-yl)oxy)ethyl)piperidine-1-carboxylate (Intermediate 643, 12.0 g, 87%) as an off-white solid, which was used for the next step without further purification. ¹H NMR (400 MHz, DMSO-d₆) δ ppm 0.99-1.08 (m, 3H), 1.38 (s, 9H), 1.59-1.67 (m, 4H), 2.57-2.77 (m, 2H), 3.89-3.92 (m, 2H), 4.33-4.42 (m, 2H), 7.00 (d, J=8.80 Hz, 1H), 8.14 (dd, J=8.74, 2.26 Hz, 1H), 8.69 (s, 1H). Mass spec m/z 276.2 [M−56+H]⁺.

### Step 2

### Intermediate 644: tert-butyl 4-(2-((5-carbamoylpyridin-2-yl)oxy)ethyl)piperidine-1-carboxylate

To a solution of tert-butyl 4-(2-((5-cyanopyridin-2-yl)oxy)ethyl)piperidine-1-carboxylate (Intermediate 643, 12.0 g, 35.49 mmol) in dimethyl sulfoxide (50 mL) was added potassium carbonate (12.2 g, 88.7 mmol) followed by 30% hydrogen peroxide (27.7 mL, 355 mmol) and the reaction mixture was stirred at room temperature for 16 h. The reaction mixture was then diluted with water (100 mL), the resultant precipitate was collected by filtration and dried in vacuo to afford tert-butyl 4-(2-((5-carbamoylpyridin-2-yl)oxy)ethyl)piperidine-1-carboxylate (Intermediate 644, 10.0 g, 80%) as a white solid, which was used for the next step without further purification. ¹H NMR (400 MHz, DMSO-d₆) δ ppm 0.90-1.08 (m, 2H), 0.94-1.05 (m, 1H), 1.34 (s, 9H), 1.55-1.67 (m, 4H), 2.56-2.73 (m, 2H), 3.78-3.89 (m, 2H), 4.26-4.32 (m, 2H), 6.81 (d, J=8.61 Hz, 1H), 7.35 (br s, 1H), 7.92 (br s, 1H), 8.06-8.08 (m, 1H), 8.62 (s, 1H). Mass spec m/z 294.2 [M−56+H]⁺.

### Step 3

### Intermediate 645: 6-(2-(piperidin-4-yl)ethoxy)nicotinamide hydrochloride

To a cooled (0° C.) solution of tert-butyl 4-(2-((5-carbamoylpyridin-2-yl)oxy)ethyl)piperidine-1-carboxylate (Intermediate 644, 10.0 g, 28.6 mmol) in dichloromethane (50 mL) was added 4M hydrochloric acid in 1,4-dioxane (100 mL) and the reaction mixture was stirred at room temperature for 3 h. The reaction mixture was then concentrated in vacuo to afford 6-(2-(piperidin-4-yl)ethoxy)nicotinamide hydrochloride (Intermediate 645, 10.0 g, 74%) as a white solid, which was used for the next step without further purification. Mass spec m/z 250.1 [M+H]⁺.

### Step 4

### Intermediate 646: 6-(2-(1-(3-bromo-2-formylphenyl)piperidin-4-yl)ethoxy)nicotinamide

To a solution of 6-(2-(piperidin-4-yl)ethoxy)nicotinamide hydrochloride (Intermediate 645, 10 g, 35.0 mmol) in dimethylformamide (70 mL) was added potassium carbonate (19.34 g, 140 mmol) followed by 2-bromo-6-fluorobenzaldehyde (CAS No. 360575-28-6, 7.10 g, 35.0 mmol) and the reaction mixture was heated at 100° C. for 16 h. The reaction mixture was then diluted with ice cold water (100 mL), the resultant precipitate was collected by filtration and dried in vacuo. The crude material was purified by combi-flash chromatography, by eluting with 10% MeOH in DCM, to afford 6-(2-(1-(3-bromo-2-formylphenyl)piperidin-4-yl)ethoxy)nicotinamide (Intermediate 646, 12.0 g, 76%) as an off-white solid. ¹H NMR (400 MHz, DMSO-d₆) δ ppm 1.33-1.50 (m, 2H), 1.52-1.85 (m, 5H), 2.76-2.95 (m, 2H), 3.13-3.20 (m, 2H), 4.35-4.42 (m, 2H), 6.85 (d, J=8.61 Hz, 1H), 7.21 (d, J=8.22 Hz, 1H), 7.26-7.46 (m, 3H), 7.95 (br s, 1H), 8.12 (dd, J=8.61, 1.96 Hz, 1H), 8.67 (br s, 1H), 10.05 (s, 1H).

### Step 5

### Intermediate 647: 6-(2-(1-(3-bromo-2-(((2,6-dioxopiperidin-3-yl)amino)methyl)phenyl)piperidin-4-yl)ethoxy)nicotinamide

To a solution of 6-(2-(1-(3-bromo-2-formylphenyl)piperidin-4-yl)ethoxy)nicotinamide (Intermediate 646, 8.0 g, 18.51 mmol) in acetonitrile (80 mL) was added 3-aminopiperidine-2,6-dione hydrochloride (CAS No. 24666-56-6, 3.35 g, 20.4 mmol) and the reaction mixture was stirred at room temperature for 2 h. Sodium cyanoborohydride (3.67 g, 55.5 mmol) was then added and the reaction mixture was stirred at room temperature for 16 h. The reaction mixture was filtered through filter paper and filtrate was concentrated in vacuo. The crude material was purified by combi-flash chromatography, by eluting with 50% ethyl acetate in heptane, to afford 6-(2-(1-(3-bromo-2-(((2,6-dioxopiperidin-3-yl)amino)methyl)phenyl)piperidin-4-yl)ethoxy)nicotinamide (Intermediate 647, 7.0 g, 69%) as an off-white solid. ¹H NMR (400 MHz, DMSO-d₆) δ ppm 0.98-1.02 (m, 1H), 1.32-1.41 (m, 2H), 1.51-1.62 (m, 1H), 1.71-1.83 (m, 4H), 1.76-1.92 (m, 1H), 1.95-1.98 (m, 1H), 2.23-2.35 (m, 1H), 2.51-2.53 (m, 3H), 2.65-2.76 (m, 1H), 3.05-3.08 (m, 1H), 3.20-3.25 (m, 1H), 3.84-3.94 (m, 2H), 4.33-4.443 (m, 2H), 6.85 (d, J=8.77 Hz, 1H), 7.10-7.20 (m, 2H), 7.31 (d, J=7.02 Hz, 1H), 7.37 (br s, 1H), 7.95 (br s, 1H), 8.11 (d, J=8.77 Hz, 1H), 8.66 (br s, 1H), 10.78 (br s, 1H). Mass spec m/z 544.2 [M+H]⁺.

### Step 6

### Intermediate 648: 6-(2-(1-(2-(2,6-dioxopiperidin-3-yl)-1-oxoisoindolin-4-yl)piperidin-4-yl)ethoxy)nicotinamide

To a solution of 6-(2-(1-(3-bromo-2-(((2,6-dioxopiperidin-3-yl)amino)methyl)phenyl)piperidin-4-yl)ethoxy)nicotinamide (Intermediate 647, 7.0 g, 12.86 mmol) in 1,4-dioxane (60 mL) was added triethylamine (5.4 mL, 38.57 mmol). The reaction mixture was purged with argon for 15 min then Xantphos (1.53 g, 2.57 mmol) followed by PdCl₂(dppf) (1.98 g, 2.57 mmol) and tungsten hexacarbonyl (2.33 g, 6.42 mmol) were added. The reaction mixture was further purged with argon for 10 min and heated at 100° C. for 16 h. The reaction mixture was then concentrated in vacuo and the crude material purified by combi-flash chromatography, by eluting with 10% MeOH in DCM, to afford 6-(2-(1-(2-(2,6-dioxopiperidin-3-yl)-1-oxoisoindolin-4-yl)piperidin-4-yl)ethoxy)nicotinamide (Intermediate 648, 4.5 g, 71%) as an off-white solid. ¹H NMR (400 MHz, DMSO-d₆) δ ppm 1.15-1.19 (m, 1H), 1.31-1.42 (m, 2H), 1.58-1.67 (m, 1H), 1.72-1.85 (m, 4H), 1.95-2.00 (m, 1H), 2.56-2.63 (m, 1H), 2.69-2.77 (m, 2H), 2.86-2.97 (m, 1H), 3.36-3.41 (m, 1H), 4.28-4.46 (m, 5H), 5.09-5.13 (m, 1H), 6.86 (d, J=8.61 Hz, 1H), 7.16 (d, J=7.83 Hz, 1H), 7.25-7.34 (m, 1H), 7.35-7.46 (m, 2H), 7.96 (br s, 1H), 8.12 (dd, J=8.41, 1.76 Hz, 1H), 8.67 (s, 1H), 10.98 (br s, 1H). Mass spec m/z 492.3 [M+H]⁺.

### Step 7

### Intermediate 649: tert-butyl 6-(6-(2-(1-(2-(2,6-dioxopiperidin-3-yl)-1-oxoisoindolin-4-yl)piperidin-4-yl)ethoxy)nicotinamido)-2-((R)-1-methylpyrrolidin-2-yl)-1H-pyrrolo[3,2-c]pyridine-1-carboxylate

To a solution of 6-(2-(1-(2-(2,6-dioxopiperidin-3-yl)-1-oxoisoindolin-4-yl)piperidin-4-yl)ethoxy)nicotinamide (Intermediate 648, 1.0 g, 2.03 mmol) and tert-butyl (R)-6-bromo-2-(1-methylpyrrolidin-2-yl)-1H-pyrrolo[3,2-c]pyridine-1-carboxylate (Intermediate 4, 0.85 g, 2.23 mmol) in 1,4-dioxane (10 mL) was added cesium carbonate (1.17 g, 6.10 mmol). The reaction mixture was purged with argon for 15 min, then Xantphos (0.36 g, 0.61 mmol) followed by Pd₂(dba)₃ (0.29 g, 0.30 mmol) were added. The reaction mixture was further purged with argon for 10 min and heated at 100° C. for 3 h. The reaction mixture was then concentrated in vacuo and the crude material purified by combi-flash chromatography, by eluting with 10% MeOH in DCM, to afford tert-butyl 6-(6-(2-(1-(2-(2,6-dioxopiperidin-3-yl)-1-oxoisoindolin-4-yl)piperidin-4-yl)ethoxy)nicotinamido)-2-((R)-1-methylpyrrolidin-2-yl)-1H-pyrrolo[3,2-c]pyridine-1-carboxylate (Intermediate 649, 1.6 g, 55%) as a yellow solid. Mass spec m/z 791.7 [M+H]⁺.

### Step 8

### Example 158: 6-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)nicotinamide

To a cooled (0° C.) solution of tert-butyl 6-(6-(2-(1-(2-(2,6-dioxopiperidin-3-yl)-1-oxoisoindolin-4-yl)piperidin-4-yl)ethoxy)nicotinamido)-2-((R)-1-methylpyrrolidin-2-yl)-1H-pyrrolo[3,2-c]pyridine-1-carboxylate (Intermediate 649, 1.60 g, 0.20 mmol) in 1,4-dioxane (5 mL) was added 4M hydrochloric acid in 1,4-dioxane (15 mL) and the reaction mixture was stirred at room temperature for 3 h. The reaction mixture was concentrated in vacuo, the residue triturated with diethyl ether (2×10 mL) and dried in vacuo. The crude material was purified by preparative HPLC (Method B) to afford 6-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)nicotinamide (Example 158, 0.06 g, 4%) as an off-white solid. ¹H NMR (400 MHz, DMSO-d₆) δ ppm 1.37-1.43 (m, 2H), 1.59-1.71 (m, 1H), 1.73-1.92 (m, 7H), 1.94-2.04 (m, 1H), 2.10-2.16 (m, 1H), 2.17 (s, 3H), 2.22-2.30 (m, 1H), 2.52-2.64 (m, 2H), 2.69-2.80 (m, 2H), 2.86-2.97 (m, 1H), 3.11-3.17 (m, 1H), 3.36-3.44 (m, 3H), 4.27-4.32 (m, 1H), 4.39-4.46 (m, 3H), 5.09-5.13 (m, 1H), 6.04 (s, 1H), 6.39 (s, 1H), 6.90 (d, J=9.01 Hz, 1H), 7.17 (d, J=7.50 Hz, 1H), 7.30 (d, J=7.00 Hz, 1H), 7.39-7.46 (m, 1H), 8.17 (s, 1H), 8.29-8.32 (m, 1H), 8.50 (s, 1H), 8.85 (s, 1H), 10.57 (br s, 1H), 11.38 (br s, 1H). Mass spec m/z 691.3 [M+H]⁺.

### Example 159 was Synthesised Following Scheme 155

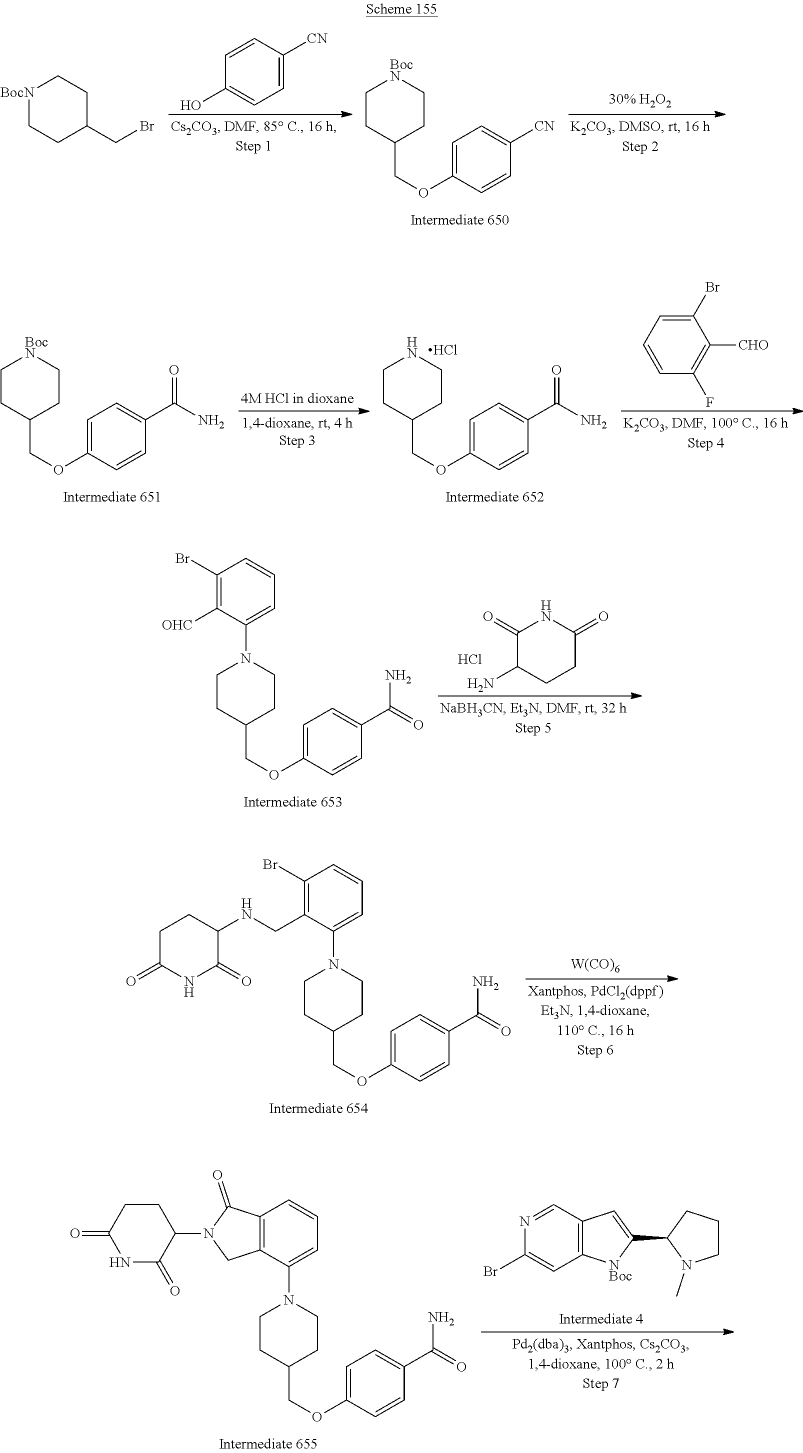

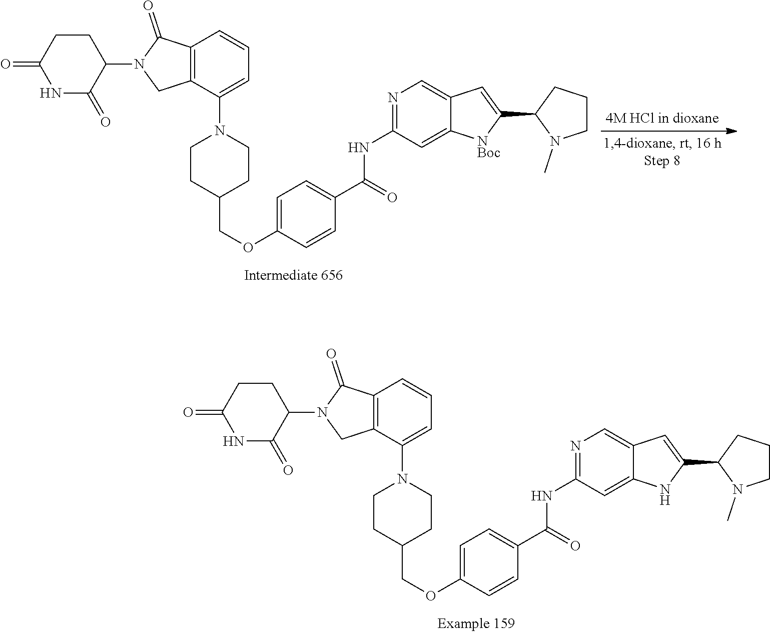 [SMILES: CN1CCC[C@@H]1c1cc2cnc(NC(=O)c3ccc(OCC4CCN(c5cccc6c5CN(C5CCC(=O)NC5=O)C6=O)CC4)cc3)cc2n1C(=O)OC(C)(C)C ; CN1CCC[C@@H]1c1cc2cnc(NC(=O)c3ccc(OCC4CCN(c5cccc6c5CN(C5CCC(=O)NC5=O)C6=O)CC4)cc3)cc2[nH]1]

### Step 1

### Intermediate 650: tert-butyl 4-((4-cyanophenoxy)methyl)piperidine-1-carboxylate

To a solution of tert-butyl 4-(bromomethyl)piperidine-1-carboxylate (CAS No. 158407-04-6, 10.0 g, 35.0 mmol) in dimethylformamide (100 mL) was added 4-hydroxybenzonitrile (CAS No 767-00-0, 4.28 g, 35.0 mmol) and cesium carbonate (35.2 g, 108 mmol) and the reaction mixture was heated at 85° C. for 16 h. The reaction mixture was then diluted with water (300 mL) and extracted with ethyl acetate (3×200 mL). The combined organic layers were dried over anhydrous Na₂SO₄ and concentrated in vacuo. The crude material was purified by combi-flash chromatography, by eluting with 90% ethyl acetate in heptane, to afford tert-butyl 4-((4-cyanophenoxy)methyl)piperidine-1-carboxylate (Intermediate 650, 6.4 g, 56%) as an off-white solid. ¹H NMR (400 MHz, DMSO-d₆) δ ppm 1.10-1.19 (m, 2H), 1.39 (s, 9H), 1.70-1.75 (m, 2H), 1.92-1.94 (m, 1H), 2.72-2.74 (m, 2H), 3.92-3.94 (m, 4H), 7.10 (d, J=8.4 Hz, 2H), 7.75 (d, J=7.6 Hz, 2H).

### Step 2

### Intermediate 651: tert-butyl 4-((4-carbamoylphenoxy)methyl)piperidine-1-carboxylate

To a cooled (0° C.) solution of tert-butyl 4-((4-cyanophenoxy)methyl)piperidine-1-carboxylate (Intermediate 650, 4.7 g, 15.0 mmol) in dimethyl sulfoxide (40 mL) were added potassium carbonate (4.1 g, 30.0 mmol) and 30% hydrogen peroxide solution (4.6 mL, 59 mmol) and the reaction mixture was stirred at room temperature for 16 h. The reaction mixture was then quenched with ice cold water (200 mL), the resultant precipitate was collected by filtration and dried in vacuo. The crude material was purified by combi-flash chromatography, by eluting with 9% MeOH in DCM, to afford tert-butyl 4-((4-carbamoylphenoxy)methyl)piperidine-1-carboxylate (Intermediate 651, 1.8 g, 36%) as white solid. ¹H NMR (400 MHz, DMSO-d₆) δ ppm 1.13-1.17 (m, 2H), 1.40 (s, 9H), 1.73-1.76 (m, 2H), 1.90-1.98 (m, 1H), 2.60-2.69 (m, 2H), 3.88-3.98 (m, 4H), 6.96 (d, J=8.4 Hz, 2H), 7.16 (bs, 1H), 7.81-7.83 (m, 3H). Mass spec m/z 279.2 [M−56+H]⁺.

### Step 3

### Intermediate 652: 4-(piperidin-4-ylmethoxy)benzamide hydrochloride

To a cooled (0° C.) solution of tert-butyl 4-((4-carbamoylphenoxy)methyl)piperidine-1-carboxylate (Intermediate 651, 1 g, 1.39 mmol) in 1,4-dioxane (20 mL) was added 4M hydrochloric acid in 1,4-dioxane (20 mL) and the reaction mixture was stirred at room temperature for 4 h. The reaction mixture was then concentrated in vacuo to afford 4-(piperidin-4-ylmethoxy)benzamide hydrochloride (Intermediate 652, 1.6 g) as yellow solid which was used for next step without further purification. ¹H NMR (400 MHz, DMSO-d₆) δ ppm 1.45-1.51 (m, 2H), 1.87-1.92 (m, 2H), 2.02-2.09 (m, 1H), 2.86-2.92 (m, 2H), 3.26-3.31 (m, 2H), 3.90-3.93 (m, 2H), 6.97 (d, J=8.4 Hz, 2H), 7.15-7.17 (m, 1H), 7.83-7.85 (m, 3H), 8.42 (br s, 1H), 8.93 (br s, 1H). Mass spec m/z 235.0 [M+H]⁺.

### Step 4

### Intermediate 653: 4-((1-(3-bromo-2-formylphenyl)piperidin-4-yl)methoxy)benzamide

To a solution of 2-bromo-6-fluorobenzaldehyde (CAS No. 360575-28-6, 1.12 g, 5.54 mmol) in dimethylformamide (15 mL) was added 4-(piperidin-4-ylmethoxy)benzamide hydrochloride (Intermediate 652, 4.28 g, 5.54 mmol) and potassium carbonate (2.29 g, 16.6 mmol) and the resulting mixture was heated at 110° C. for 16 h. The reaction mixture was then diluted with water (500 mL) and extracted with ethyl acetate (3×200 mL). The combined organic layers were dried over anhydrous Na₂SO₄ and concentrated in vacuo to obtain the crude product. The crude material was purified by combi-flash chromatography, by eluting with 9% MeOH in DCM, to afford 4-((1-(3-bromo-2-formylphenyl)piperidin-4-yl)methoxy)benzamide (Intermediate 653, 1.6 g, 69%) as a yellow solid. ¹H NMR (400 MHz, DMSO-d₆) δ ppm 1.47-1.56 (m, 2H), 1.86-1.94 (m, 3H), 2.73-2.76 (m, 2H), 3.21-3.24 (m, 2H), 3.95-3.97 (m, 2H), 6.98-7.00 (m, 2H), 7.19 (bs, 1H), 7.24 (d, J=8.0 Hz, 1H), 7.32 (d, J=8.0 Hz, 1H), 7.43 (d, J=8.0 Hz, 1H), 7.83-7.85 (m, 3H), 10.05 (s, 1H).

### Step 5

### Intermediate 654: 4-((1-(3-bromo-2-(((2,6-dioxopiperidin-3-yl)amino)methyl)phenyl)piperidin-4-yl)methoxy)benzamide

To a solution of 4-((1-(3-bromo-2-formylphenyl)piperidin-4-yl)methoxy)benzamide (Intermediate 653, 1.6 g, 3.8 mmol) in dimethylformamide (25 mL) was added 3-aminopiperidine-2,6-dione hydrochloride (CAS No. 24666-56-6, 0.76 g, 4.6 mmol) and triethylamine (2.1 mL, 15 mmol) and the reaction mixture was heated at 70° C. for 16 h. Sodium cyanoborohydride (1.5 g, 23 mmol) was then added and the reaction mixture was heated at 70° C. for 16 h. The reaction mixture was filtered through a celite bed and the filter pad was washed with dichloromethane (100 mL). The filtrate was concentrated in vacuo to obtain the crude product. The crude compound was purified by combi-flash chromatography, by eluting with 8% MeOH in DCM, to afford 4-((1-(3-bromo-2-(((2,6-dioxopiperidin-3-yl)amino)methyl)phenyl)piperidin-4-yl)methoxy)benzamide (Intermediate 654, 1.8 g, 89%) as a yellow solid. ¹H NMR (400 MHz, DMSO-d₆) δ ppm 1.47-1.53 (m, 2H), 1.67-1.76 (m, 1H), 1.82-1.89 (m, 3H), 2.28-2.38 (m, 1H), 2.53-2.60 (m, 3H), 2.64-2.98 (m, 4H), 3.10-3.29 (m, 2H), 3.88-3.96 (m, 3H), 6.97-7.00 (m, 2H), 7.13-7.20 (m, 3H), 7.33-7.35 (m, 1H), 7.83-7.85 (m, 3H), 10.79 (bs, 1H). Mass spec m/z 529.0 [M+H]⁺.

### Step 6

### Intermediate 655: 4-((1-(2-(2,6-dioxopiperidin-3-yl)-1-oxoisoindolin-4-yl)piperidin-4-yl)methoxy)benzamide

To a solution of 4-((1-(3-bromo-2-(((2,6-dioxopiperidin-3-yl)amino)methyl)phenyl)piperidin-4-yl)methoxy)benzamide (Intermediate 654, 1.7 g, 3.2 mmol) in 1,4-dioxane (20 mL) was added triethylamine (1.3 mL, 9.6 mmol) and the reaction mixture was purged with argon for 15 min. Then Xanthphos (0.38 g, 0.64 mmol), PdCl₂(dppf) (0.49 g, 0.64 mmol) and WO(CO)₆ (0.58 g, 1.6 mmol) were added, and the reaction mixture was purged with argon for another 10 min and stirred at 110° C. for 16 h. The reaction mixture was then filtered through a celite bed and the filter pad was washed with ethyl acetate (100 mL). The filtrate was concentrated in vacuo to obtain the crude product. The crude material was purified by combi-flash chromatography, by eluting with 9% MeOH in DCM, to afford tert-butyl 4-((1-(2-(2,6-dioxopiperidin-3-yl)-1-oxoisoindolin-4-yl)piperidin-4-yl)methoxy)benzamide (Intermediate 655, 0.70 g, 46%) as a yellow solid. ¹H NMR (400 MHz, DMSO-d₆) δ ppm 1.44-1.53 (m, 2H), 1.88-2.01 (m, 4H), 2.54-2.67 (m, 1H), 2.91-2.96 (m, 1H), 3.08-3.17 (m, 3H), 3.37-3.46 (m, 2H), 3.95-3.97 (m, 2H), 4.28-4.33 (m, 1H), 4.42-4.47 (m, 1H), 5.09-5.14 (m, 1H), 6.98-7.00 (m, 2H), 7.15-7.20 (m, 2H), 7.30 (d, J=7.6 Hz, 1H), 7.44 (t, J=7.6 Hz, 1H), 7.82-7.85 (m, 3H), 10.79 (bs, 1H). Mass spec m/z 477.3 [M+H]⁺.

### Step 7

### Intermediate 656: tert-butyl 6-(4-((1-(2-(2,6-dioxopiperidin-3-yl)-1-oxoisoindolin-4-yl)piperidin-4-yl)methoxy)benzamido)-2-((R)-1-methylpyrrolidin-2-yl)-1H-pyrrolo[3,2-c]pyridine-1-carboxylate

To a solution of 4-((1-(2-(2,6-dioxopiperidin-3-yl)-1-oxoisoindolin-4-yl)piperidin-4-yl)methoxy)benzamide (Intermediate 655, 0.47 g, 0.98 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 0.45 g, 1.18 mmol) and cesium carbonate (0.96 g, 0.29 mmol). The reaction mixture was purged with argon for 15 min and then Xanthphos (0.17 g, 0.29 mmol) and Pd₂(dba)₃ (0.14 g, 0 0.14 mmol) were added. The reaction mixture was purged with argon for further 10 min and heated at 100° C. for 2 h. The reaction mixture was filtered through a celite bed, and the filter pad was washed with ethyl acetate (100 mL). The filtrate was concentrated in vacuo to obtain the crude product. The crude material was purified by combi-flash chromatography, by eluting with 90% ethyl acetate in heptane, to afford tert-butyl 6-(4-((1-(2-(2,6-dioxopiperidin-3-yl)-1-oxoisoindolin-4-yl)piperidin-4-yl)methoxy)benzamido)-2-((R)-1-methylpyrrolidin-2-yl)-1H-pyrrolo[3,2-c]pyridine-1-carboxylate (Intermediate 656, 0.42 g, 55%) as a yellow solid. ¹H NMR (400 MHz, DMSO-d₆) δ ppm 1.34-1.50 (m, 4H), 1.64-1.68 (m, 12H), 1.88-1.97 (m, 5H), 2.27-2.35 (m, 4H), 2.73-2.90 (m, 3H), 3.36-3.42 (m, 3H), 3.90-4.01 (m, 3H), 4.27-4.32 (m, 1H), 4.42-4.46 (m, 1H), 5.09-5.12 (m, 1H), 6.73 (bs, 1H), 7.02-7.04 (m, 2H), 7.16-7.18 (m, 1H), 7.28-7.30 (m, 1H), 7.40-7.42 (m, 1H), 8.02-8.04 (m, 2H), 8.57 (bs, 1H), 8.90 (bs, 1H), 10.51 (bs, 1H), 10.78 (bs, 1H). Mass spec m/z 776.3 [M+H]⁺.

### Step 8

### Example 159: 4-((1-(2-(2,6-dioxopiperidin-3-yl)-1-oxoisoindolin-4-yl)piperidin-4-yl)methoxy)-N-(2-((R)-1-methylpyrrolidin-2-yl)-1H-pyrrolo[3,2-c]pyridin-6-yl)benzamide

To a cooled (0° C.) solution of tert-butyl 6-(4-((1-(2-(2,6-dioxopiperidin-3-yl)-1-oxoisoindolin-4-yl)piperidin-4-yl)methoxy)benzamido)-2-((R)-1-methylpyrrolidin-2-yl)-1H-pyrrolo[3,2-c]pyridine-1-carboxylate (Intermediate 656, 0.38 g, 0.49 mmol) in 1,4-dioxane (5 mL) was added 4M hydrochloric acid in 1,4-dioxane (5 mL) and the reaction mixture was stirred at room temperature for 16 h. The reaction mixture was concentrated in vacuo. The crude material was purified by preparative HPLC (Method A) to afford 4-((1-(2-(2,6-dioxopiperidin-3-yl)-1-oxoisoindolin-4-yl)piperidin-4-yl)methoxy)-N-(2-((R)-1-methylpyrrolidin-2-yl)-1H-pyrrolo[3,2-c]pyridin-6-yl)benzamide (Example 159, 0.04 g, 15%) as an off-white solid. ¹H NMR (400 MHz, DMSO-d₆) δ ppm 1.43-1.54 (m, 2H), 1.76-2.03 (m, 7H), 2.11-2.18 (m, 4H), 2.23-2.77 (m, 1H), 2.56-2.61 (m, 1H), 2.73-2.92 (m, 3H), 3.12-3.17 (m, 1H), 3.29-3.34 (m, 2H), 3.40-3.49 (m, 2H), 3.99-4.01 (m, 2H), 4.29-4.31 (m, 1H), 4.43-4.48 (m, 1H), 5.07-5.12 (m, 1H), 6.38 (s, 1H), 7.03-7.06 (m, 2H), 7.18 (d, J=8.4 Hz, 1H), 7.31 (d, J=8.4 Hz, 1H), 7.43 (d, J=8.4 Hz, 1H), 8.05 (d, J=8.4 Hz, 2H), 8.17 (s, 1H), 8.49 (s, 1H), 10.26 (s, 1H), 10.98 (bs, 1H), 11.33 (s, 1H). Mass spec m/z 676.4 [M+H]⁺.

### Example 160 was Synthesised Following Scheme 156

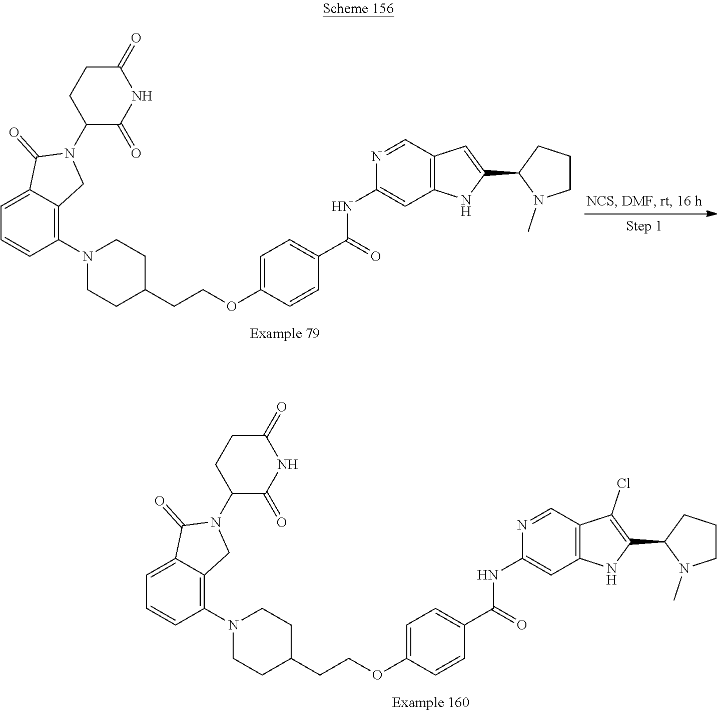 [SMILES: CN1CCC[C@@H]1c1cc2cnc(NC(=O)c3ccc(OCCC4CCN(c5cccc6c5CN(C5CCC(=O)NC5=O)C6=O)CC4)cc3)cc2[nH]1 ; CN1CCC[C@@H]1c1[nH]c2cc(NC(=O)c3ccc(OCCC4CCN(c5cccc6c5CN(C5CCC(=O)NC5=O)C6=O)CC4)cc3)ncc2c1Cl]

### Step 1

### Example 160: N-(3-chloro-2-((R)-1-methylpyrrolidin-2-yl)-1H-pyrrolo[3,2-c]pyridin-6-yl)-4-(2-(1-(2-(2,6-dioxopiperidin-3-yl)-1-oxoisoindolin-4-yl)piperidin-4-yl)ethoxy)benzamide

To a solution of 4-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide (Example 79, 0.18 g, 0.26 mmol) in dimethylformamide (5 mL) was added N-chlorosuccinimide (0.07 g, 0.52 mmol) and the reaction mixture was stirred at room temperature for 16 h. The reaction mixture was diluted with ice cold water (20 mL) and extracted with ethyl acetate (3×20 mL). The combined organic layers were dried over anhydrous Na₂SO₄ and concentrated in vacuo. The crude material was purified by preparative HPLC (Method B) to afford N-(3-chloro-2-((R)-1-methylpyrrolidin-2-yl)-1H-pyrrolo[3,2-c]pyridin-6-yl)-4-(2-(1-(2-(2,6-dioxopiperidin-3-yl)-1-oxoisoindolin-4-yl)piperidin-4-yl)ethoxy)benzamide (Example 160, 0.02 g, 11%) as off-white solid. ¹H NMR (400 MHz, DMSO-d₆) δ ppm 1.38-1.46 (m, 2H), 1.65-1.72 (m, 1H), 1.74-1.79 (m, 2H), 1.81-1.91 (m, 4H), 1.93-2.03 (m, 2H), 2.16 (s, 3H), 2.16-2.20 (m, 1H), 2.29-2.34 (m, 1H), 2.54-2.63 (m, 2H), 2.70-2.80 (m, 2H), 2.87-2.97 (m, 1H), 3.15-3.19 (m, 1H), 3.36-3.44 (m, 2H), 3.49-3.55 (m, 1H), 4.13-4.16 (m, 2H), 4.26-4.34 (m, 1H), 4.40-4.48 (m, 1H), 5.09-5.13 (m, 1H), 7.04 (d, J=8.88 Hz, 2H), 7.18 (d, J=7.75 Hz, 1H), 7.30 (d, J=7.38 Hz, 1H), 7.40-7.46 (m, 1H), 8.05 (d, J=8.88 Hz, 2H), 8.23 (s, 1H), 8.49 (s, 1H), 10.45 (br s, 1H), 10.98 (br s, 1H), 11.67 (br s, 1H). Mass spec m/z 724.4 [M+H]⁺.

### Examples 161 and 162 were Prepared Following Scheme 157

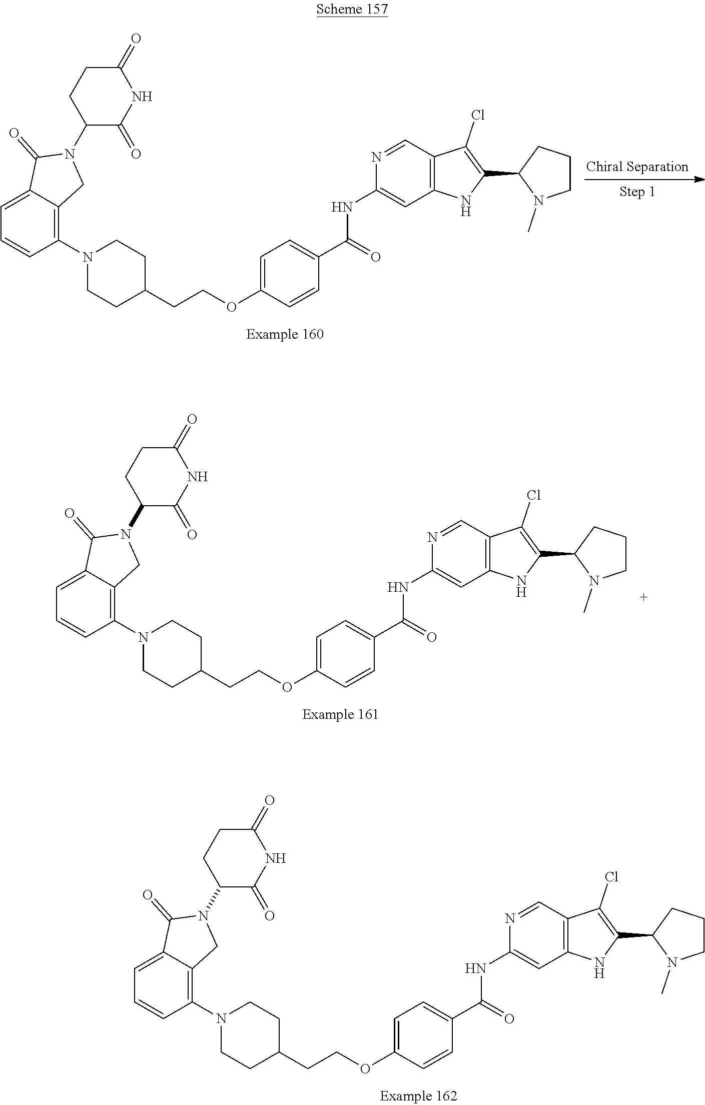

### Step 1

### Example 161: N-(3-chloro-2-((R)-1-methylpyrrolidin-2-yl)-1H-pyrrolo[3,2-c]pyridin-6-yl)-4-(2-(1-(2-((rel-S)-2,6-dioxopiperidin-3-yl)-1-oxoisoindolin-4-yl)piperidin-4-yl)ethoxy)benzamide and Example 162: N-(3-chloro-2-((R)-1-methylpyrrolidin-2-yl)-1H-pyrrolo[3,2-c]pyridin-6-yl)-4-(2-(1-(2-((rel-R)-2,6-dioxopiperidin-3-yl)-1-oxoisoindolin-4-yl)piperidin-4-yl)ethoxy)benzamide

N-(3-chloro-2-((R)-1-methylpyrrolidin-2-yl)-1H-pyrrolo[3,2-c]pyridin-6-yl)-4-(2-(1-(2-(2,6-dioxopiperidin-3-yl)-1-oxoisoindolin-4-yl)piperidin-4-yl)ethoxy)benzamide (Example 160, 0.205 g, 0.28 mmol) was separated by chiral preparative HPLC (Column: Chiral Pak IC (20×250*mm), 5 μm; Mobile Phase A: DCM, Mobile Phase B: iPrOH (1:1); Flow rate: 20 ml/min; isocratic 50% B) to afford N-(3-chloro-2-((R)-1-methylpyrrolidin-2-yl)-1H-pyrrolo[3,2-c]pyridin-6-yl)-4-(2-(1-(2-((rel-S)-2,6-dioxopiperidin-3-yl)-1-oxoisoindolin-4-yl)piperidin-4-yl)ethoxy)benzamide (Example 161, 1st eluting peak, 0.045 g, 22%) as an off-white solid and N-(3-chloro-2-((R)-1-methylpyrrolidin-2-yl)-1H-pyrrolo[3,2-c]pyridin-6-yl)-4-(2-(1-(2-((rel-R)-2,6-dioxopiperidin-3-yl)-1-oxoisoindolin-4-yl)piperidin-4-yl)ethoxy)benzamide (Example 162, 2nd eluting peak, 0.046 g, 22%) as an off-white solid.

### Example 161: N-(3-chloro-2-((R)-1-methylpyrrolidin-2-yl)-1H-pyrrolo[3,2-c]pyridin-6-yl)-4-(2-(1-(2-((rel-S)-2,6-dioxopiperidin-3-yl)-1-oxoisoindolin-4-yl)piperidin-4-yl)ethoxy)benzamide

1^{st} eluting peak from Chiral preparative HPLC. Retention time: 11.23 min.

¹H NMR (400 MHz, DMSO-d₆) δ ppm 1.35-1.42 (m, 2H), 1.61-1.72 (m, 1H), 1.73-1.78 (m, 2H), 1.82-1.88 (m, 4H), 1.91-2.00 (m, 2H), 2.16 (s, 3H), 2.17-2.22 (m, 1H), 2.26-2.34 (m, 1H), 2.55-2.63 (m, 2H), 2.65-2.81 (m, 2H), 2.85-2.97 (m, 1H), 3.15-3.18 (m, 1H), 3.35-3.45 (m, 2H), 3.49-33.53 (m, 1H), 4.11-4.18 (m, 2H), 4.27-4.31 (m, 1H), 4.41-4.46 (m, 1H), 5.09-5.13 (m, 1H), 7.04 (d, J=8.22 Hz, 2H), 7.17 (d, J=7.83 Hz, 1H), 7.30 (d, J=7.04 Hz, 1H), 7.43 (t, J=7.63 Hz, 1H), 8.05 (d, J=8.61 Hz, 2H), 8.23 (s, 1H), 8.48 (s, 1H), 10.45 (br s, 1H), 10.98 (br s, 1H), 11.66 (br s, 1H). Mass spec m/z 724.3 [M+H]⁺.

### Example 162: N-(3-chloro-2-((R)-1-methylpyrrolidin-2-yl)-1H-pyrrolo[3,2-c]pyridin-6-yl)-4-(2-(1-(2-((rel-R)-2,6-dioxopiperidin-3-yl)-1-oxoisoindolin-4-yl)piperidin-4-yl)ethoxy)benzamide

2nd eluting peak from Chiral preparative HPLC. Retention time: 17.02 min.

¹H NMR (400 MHz, DMSO-d₆) δ ppm 1.36-1.42 (m, 2H), 1.61-1.72 (m, 1H), 1.73-1.78 (m, 2H), 1.82-1.88 (m, 4H), 1.91-2.00 (m, 2H), 2.16 (s, 3H), 2.17-2.22 (m, 1H), 2.27-2.34 (m, 1H), 2.56-2.63 (m, 2H), 2.65-2.81 (m, 2H), 2.85-2.97 (m, 1H), 3.15-3.18 (m, 1H), 3.35-3.45 (m, 2H), 3.49-33.53 (m, 1H), 4.13-4.16 (m, 2H), 4.27-4.32 (m, 1H), 4.42-4.46 (m, 1H), 5.09-5.13 (m, 1H), 7.04 (d, J=8.22 Hz, 2H), 7.17 (d, J=7.83 Hz, 1H), 7.30 (d, J=7.04 Hz, 1H), 7.43 (t, J=7.63 Hz, 1H), 8.05 (d, J=8.61 Hz, 2H), 8.23 (s, 1H), 8.49 (s, 1H), 10.45 (br s, 1H), 10.96 (br s, 1H), 11.66 (br s, 1H). Mass spec m/z 724.2 [M+H]⁺.

### Example 163 was Synthesised Following Scheme 158

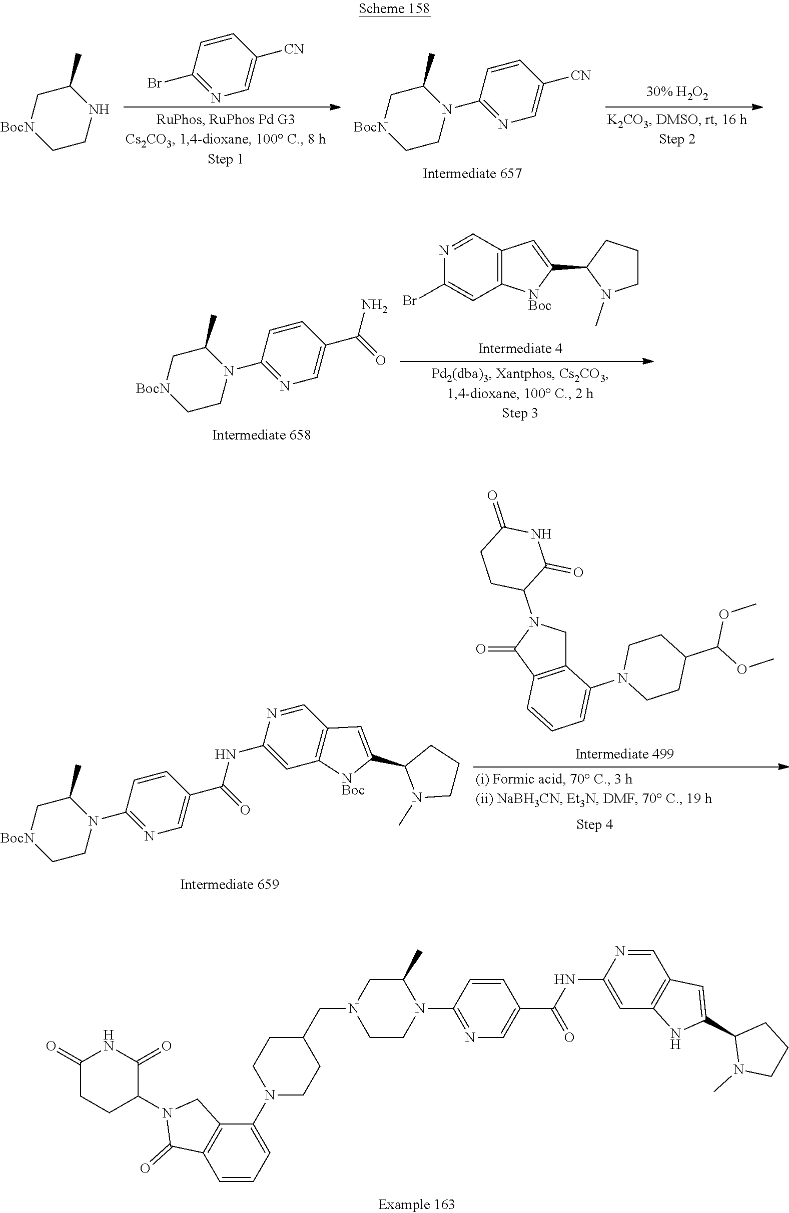

### Step 1

### Intermediate 657: tert-butyl (R)-4-(5-cyanopyridin-2-yl)-3-methylpiperazine-1-carboxylate

To a solution of tert-butyl (R)-3-methylpiperazine-1-carboxylate (CAS No. 163765-44-4, 3.0 g, 14.5 mmol) in 1,4-dioxane (15 mL) were added 2-bromo-5-cyanopyridine (CAS No. 139585-70-9, 3.02 g, 16.0 mmol) and cesium carbonate (14.2 g, 43.6 mmol). The reaction mixture was purged with argon for 15 min then RuPhos (1.38 g, 2.91 mmol) and RuPhos Pd G3 (1.28 g, 1.45 mmol) were added. The reaction mixture was purged with argon for further 10 min and heated at 100° C. for 8 h then concentrated in vacuo. The crude material was purified by combi-flash chromatography, by eluting with 3% MeOH in DCM, to afford tert-butyl (R)-4-(5-cyanopyridin-2-yl)-3-methylpiperazine-1-carboxylate (Intermediate 657, 2.40 g, 46%) as pale yellow solid. ¹H NMR (400 MHz, DMSO-d₆) δ ppm 1.05-1.09 (m, 3H), 1.42 (s, 9H), 2.86-3.03 (m, 1H), 3.10-3.20 (m, 2H), 3.75-3.82 (m, 1H), 3.87-3.98 (m, 1H), 4.10-4.19 (m, 1H), 4.55-4.64 (m, 1H), 6.88 (d, J=9.00 Hz, 1H), 7.88 (dd, J=9.00, 1.57 Hz, 1H), 8.51 (s, 1H). Mass spec m/z 303.1 [M+H]⁺.

### Step 2

### Intermediate 658: tert-butyl (R)-4-(5-carbamoylpyridin-2-yl)-3-methylpiperazine-1-carboxylate

To a solution of tert-butyl (R)-4-(5-cyanopyridin-2-yl)-3-methylpiperazine-1-carboxylate (Intermediate 657, 2.0 g, 6.61 mmol) in dimethyl sulfoxide (20 mL) was added potassium carbonate (2.28 g, 16.5 mmol) followed by 30% hydrogen peroxide (5.17 mL, 66.1 mmol) and the reaction mixture was stirred at room temperature for 16 h. The reaction mixture was diluted with water (100 mL), the resultant precipitate was collected by filtration and dried in vacuo to afford tert-butyl (R)-4-(5-carbamoylpyridin-2-yl)-3-methylpiperazine-1-carboxylate (Intermediate 658, 1.80 g, 85%) as an off-white solid, which was used for the next step without further purification. ¹H NMR (400 MHz, DMSO-d₆) δ ppm 1.01-1.05 (m, 3H), 1.40 (s, 9H), 2.79-3.15 (m, 3H), 3.72-3.80 (m, 1H), 3.85-4.00 (m, 1H), 4.04-4.14 (m, 1H), 4.51-4.61 (m, 1H), 6.76 (d, J=9.00 Hz, 1H), 7.11 (br s, 1H), 7.73 (br s, 1H), 7.95 (d, J=8.61 Hz, 1H), 8.60 (s, 1H). Mass spec m/z 321.1 [M+H]⁺.

### Step 3

### Intermediate 659: tert-butyl 6-(6-((R)-4-(tert-butoxycarbonyl)-2-methylpiperazin-1-yl)nicotinamido)-2-((R)-1-methylpyrrolidin-2-yl)-1H-pyrrolo[3,2-c]pyridine-1-carboxylate

To a solution of tert-butyl (R)-4-(5-carbamoylpyridin-2-yl)-3-methylpiperazine-1-carboxylate (Intermediate 658, 0.50 g, 1.56 mmol) in 1,4-dioxane (8 mL) was added tert-butyl (R)-6-bromo-2-(1-methylpyrrolidin-2-yl)-1H-pyrrolo[3,2-c]pyridine-1-carboxylate (Intermediate 4, 0.65 g, 1.71 mmol) followed by cesium carbonate (1.52 g, 4.68 mmol). The reaction mixture was purged with argon for 15 min, then Xantphos (0.13 g, 0.23 mmol) and Pd₂(dba)₃ (0.22 g, 0.23 mmol) were added. The reaction mixture was purged with argon for another 10 min and heated at 100° C. for 2 h then concentrated in vacuo. The crude material was purified by combi-flash chromatography, by eluting with 100% ethyl acetate, to afford tert-butyl 6-(6-((R)-4-(tert-butoxycarbonyl)-2-methylpiperazin-1-yl)nicotinamido)-2-((R)-1-methylpyrrolidin-2-yl)-1H-pyrrolo[3,2-c]pyridine-1-carboxylate (Intermediate 659, 0.6 g, 62%) as an off yellow solid. ¹H NMR (401 MHz, DMSO-d₆) δ ppm 1.06-1.11 (m, 3H), 1.43 (s, 9H), 1.56-1.67 (m, 2H), 1.69 (s, 9H), 1.71-1.79 (m, 1H), 2.30-2.35 (m, 2H), 2.36 (s, 3H), 2.90-3.02 (m, 1H), 3.05-3.19 (m, 3H), 3.78-3.86 (m, 1H), 3.88-3.94 (m, 1H), 4.00-4.07 (m, 1H), 4.14-4.21 (m, 1H), 4.60-4.67 (m, 1H), 6.74 (s, 1H), 6.84 (d, J=9.54 Hz, 1H), 8.15-8.21 (m, 1H), 8.58 (s, 1H), 8.82 (s, 1H), 8.91 (s, 1H), 10.56 (br s, 1H). Mass spec m/z 620.3 [M+H]⁺.

### Step 4

### Example 163: 6-((2R)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)nicotinamide

A solution of 3-(4-(4-(dimethoxymethyl)piperidin-1-yl)-1-oxoisoindolin-2-yl)piperidine-2,6-dione (Intermediate 499, 0.5 g, 1.24 mmol) in formic acid (1.0 mL) was heated at 70° C. for 3 h. The reaction mixture was concentrated in vacuo. A solution of tert-butyl 6-(6-((R)-4-(tert-butoxycarbonyl)-2-methylpiperazin-1-yl)nicotinamido)-2-((R)-1-methylpyrrolidin-2-yl)-1H-pyrrolo[3,2-c]pyridine-1-carboxylate (Intermediate 659, 0.77 g, 1.24 mmol) in dimethylformamide (3 mL) followed by triethylamine (0.87 mL 6.22 mmol) were added and the reaction mixture was heated at 70° C. for 3 h. Sodium cyanoborohydride (0.33 g, 4.98 mmol) was then added and the reaction heated at 70° C. for 16 h. The reaction mixture was poured into ice cold water (30 mL), the resultant precipitate was collected by filtration and dried in vacuo. The crude material was purified purified by preparative HPLC (Method B) to afford 6-((2R)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)nicotinamide (Example 163, 0.11 g, 12%) as an off-white solid. ¹H NMR (400 MHz, DMSO-d₆) δ ppm 1.18-1.23 (m, 3H), 1.26-1.35 (m, 2H), 1.77-1.93 (m, 6H), 1.96-2.05 (m, 2H), 2.10-2.15 (m, 1H), 2.16 (s, 3H), 2.18-2.30 (m, 4H), 2.55-2.63 (m, 2H), 2.70-2.84 (m, 3H), 2.86-2.98 (m, 2H), 3.04-3.18 (m, 2H), 3.26-3.30 (m, 1H), 3.36-3.47 (m, 2H), 4.14-4.23 (m, 1H), 4.26-4.35 (m, 1H), 4.40-4.48 (m, 1H), 4.56-4.64 (m, 1H), 5.09-5.14 (m, 1H), 6.37 (s, 1H), 6.81 (d, J=9.13 Hz, 1H), 7.17 (d, J=7.88 Hz, 1H), 7.29 (d, J=7.38 Hz, 1H), 7.41-7.47 (m, 1H), 8.16-8.20 (m, 2H), 8.48 (s, 1H), 8.80 (s, 1H), 10.26 (br s, 1H), 10.98 (br s, 1H), 11.33 (br s, 1H). Mass spec m/z 759.3 [M+H]⁺.

### Example 164 was Synthesised Following Scheme 159

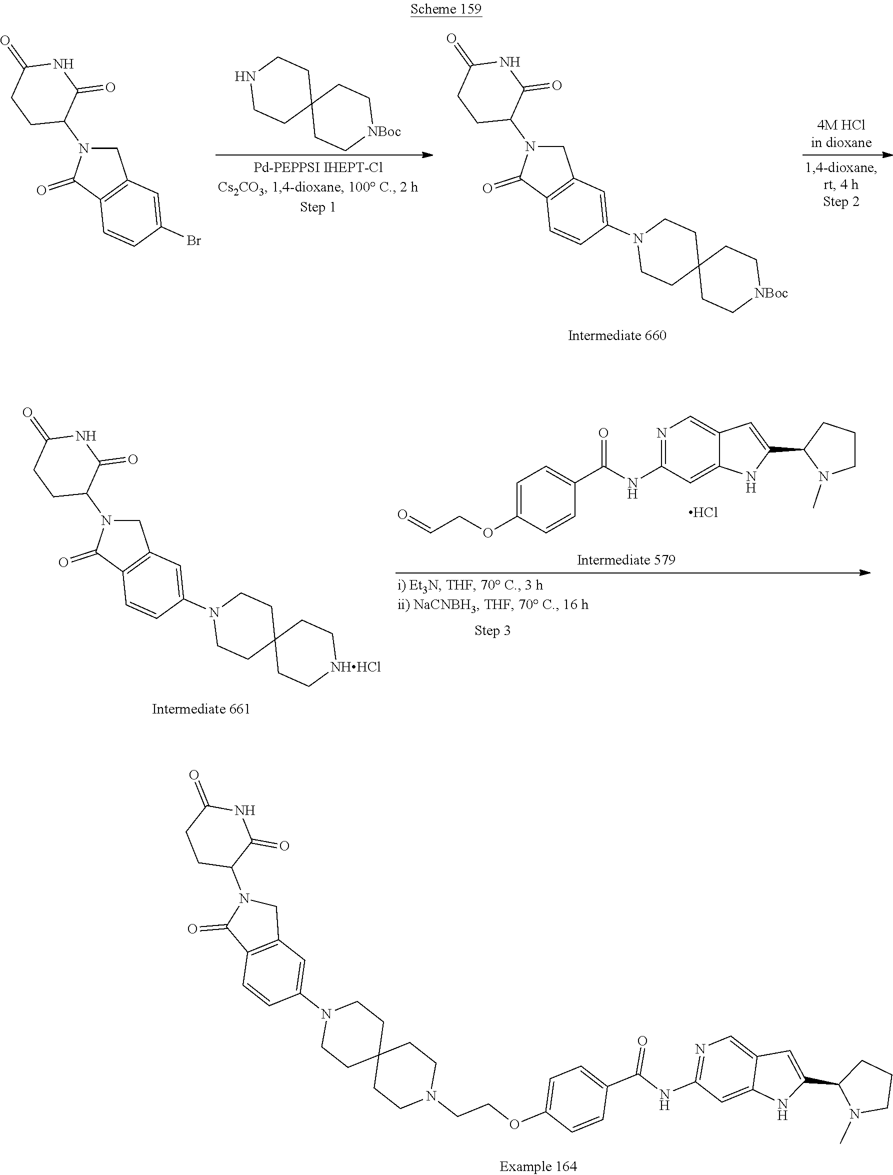

### Step 1

### Intermediate 660: tert-butyl 9-(2-(2,6-dioxopiperidin-3-yl)-1-oxoisoindolin-5-yl)-3,9-diazaspiro[5.5]undecane-3-carboxylate

To a solution of 3-(5-bromo-1-oxoisoindolin-2-yl)piperidine-2,6-dione (CAS No. 1010100-26-1, 2.0 g, 6.19 mmol) in 1,4-dioxane (20 mL) was added tert-butyl 3,9-diazaspiro[5.5]undecane-3-carboxylate (CAS No, 173405-78-2, 1.57 g, 6.19 mmol) and cesium carbonate (6.08 g, 18.6 mmol). The reaction mixture was purged with argon for 15 min, then Pd-PEPPSI-IHEPT-C₁ (1.26 g, 1.23 mmol) was added. The reaction mixture was further purged with argon for 10 min and heated at 100° C. for 2 h. After completion, the reaction mixture was concentrated in vacuo. The crude material was purified by combi-flash chromatography, by eluting with 90% ethyl acetate in heptane, to afford tert-butyl 9-(2-(2,6-dioxopiperidin-3-yl)-1-oxoisoindolin-5-yl)-3,9-diazaspiro[5.5]undecane-3-carboxylate (Intermediate 660, 1.9 g, 62%) as a white solid. ¹H NMR (400 MHz, DMSO-d₆) δ ppm 0.70-0.90 (m, 3H), 1.16-1.22 (m, 3H), 1.38 (s, 9H), 1.39-1.49 (m, 5H), 1.49-1.58 (m, 4H), 1.89-2.00 (m, 1H), 2.27-2.39 (m, 1H), 2.79-2.96 (m, 1H), 3.12-3.21 (m, 2H), 4.11-4.25 (m, 1H), 4.15-4.30 (m, 1H), 5.00-5.04 (m, 1H), 7.03 (s, 1H), 7.39-7.56 (m, 1H), 7.92 (s, 1H), 10.91 (br s, 1H). Mass spec m/z 497.3 [M+H]⁺.

### Step 2

### Intermediate 661: 3-(1-oxo-5-(3,9-diazaspiro[5.5]undecan-3-yl)isoindolin-2-yl)piperidine-2,6-dione hydrochloride

To a cooled (0° C.) solution of tert-butyl 9-(2-(2,6-dioxopiperidin-3-yl)-1-oxoisoindolin-5-yl)-3,9-diazaspiro[5.5]undecane-3-carboxylate (Intermediate 660, 1.90 g, 3.80 mmol) in 1,4-dioxane (20 mL) was added 4M hydrochloric acid in 1,4-dioxane (20 mL, 80 mmol). The reaction mixture was stirred at room temperature for 4 h. After completion, the reaction mixture was concentrated in vacuo to afford 3-(1-oxo-5-(3,9-diazaspiro[5.5]undecan-3-yl)isoindolin-2-yl)piperidine-2,6-dione hydrochloride (Intermediate 661, 2.0 g) as a white solid which was used for the next step without further purification. Mass spec m/z 397.1 [M+H]⁺.

### Step 3

### Example 164: 4-(2-(9-(2-(2,6-dioxopiperidin-3-yl)-1-oxoisoindolin-5-yl)-3,9-diazaspiro[5.5]undecan-3-yl)ethoxy)-N-(2-((R)-1-methylpyrrolidin-2-yl)-1H-pyrrolo[3,2-c]pyridin-6-yl)benzamide

To a solution of (R)-2-(1-methylpyrrolidin-2-yl)-6-(4-(2-oxoethoxy)benzamido)-1H-pyrrolo[3,2-c]pyridine-1-carboxylate hydrochloride (Intermediate 579, 0.1 g, 0.24 mmol) in tetrahydrofuran (2 mL) were added 3-(1-oxo-5-(3,9-diazaspiro[5.5]undecan-3-yl)isoindolin-2-yl)piperidine-2,6-dione hydrochloride (Intermediate 661, 0.12 g, 0.28 mmol) and triethylamine (0.10 mL, 0.72 mmol) and the reaction mixture was heated at 70° C. for 3 h. Sodium cyanoborohydride (0.06 g, 0.96 mmol) was then added and the reaction mixture was heated at 70° C. for 16 h. The reaction mixture was diluted with water (30 mL), the resultant precipitate was collected by filtration and dried in vacuo. The crude material was purified by preparative HPLC (Method B) to afford 4-(2-(9-(2-(2,6-dioxopiperidin-3-yl)-1-oxoisoindolin-5-yl)-3,9-diazaspiro[5.5]undecan-3-yl)ethoxy)-N-(2-((R)-1-methylpyrrolidin-2-yl)-1H-pyrrolo[3,2-c]pyridin-6-yl)benzamide (Example 164, 0.01 g, 5%) as an off-white solid. ¹H NMR (400 MHz, DMSO-d₆) δ ppm 1.46-1.57 (m, 8H), 1.83-1.99 (m, 5H), 2.10-2.15 (m, 1H), 2.16 (s, 3H), 2.22-2.28 (m, 1H), 2.36-2.39 (m, 1H), 2.42-2.46 (m, 2H), 2.54-2.58 (m, 2H), 2.58-2.62 (m, 1H), 2.70-2.76 (m, 2H), 2.84-2.95 (m, 1H), 3.12-3.17 (m, 1H), 3.24-3.31 (m, 4H), 4.15-4.22 (m, 3H), 4.28-4.35 (m, 1H), 5.01-5.06 (m, 1H), 6.38 (s, 1H), 7.02-7.07 (m, 4H), 7.49 (d, J=9.13 Hz, 1H), 8.04 (d, J=8.88 Hz, 2H), 8.17 (s, 1H), 8.48 (s, 1H), 10.27 (br s, 1H), 10.93 (br s, 1H), 11.34 (br s, 1H). Mass spec m/z 759.7 [M+H]⁺.

### Example 165 was Synthesised Following Scheme 160

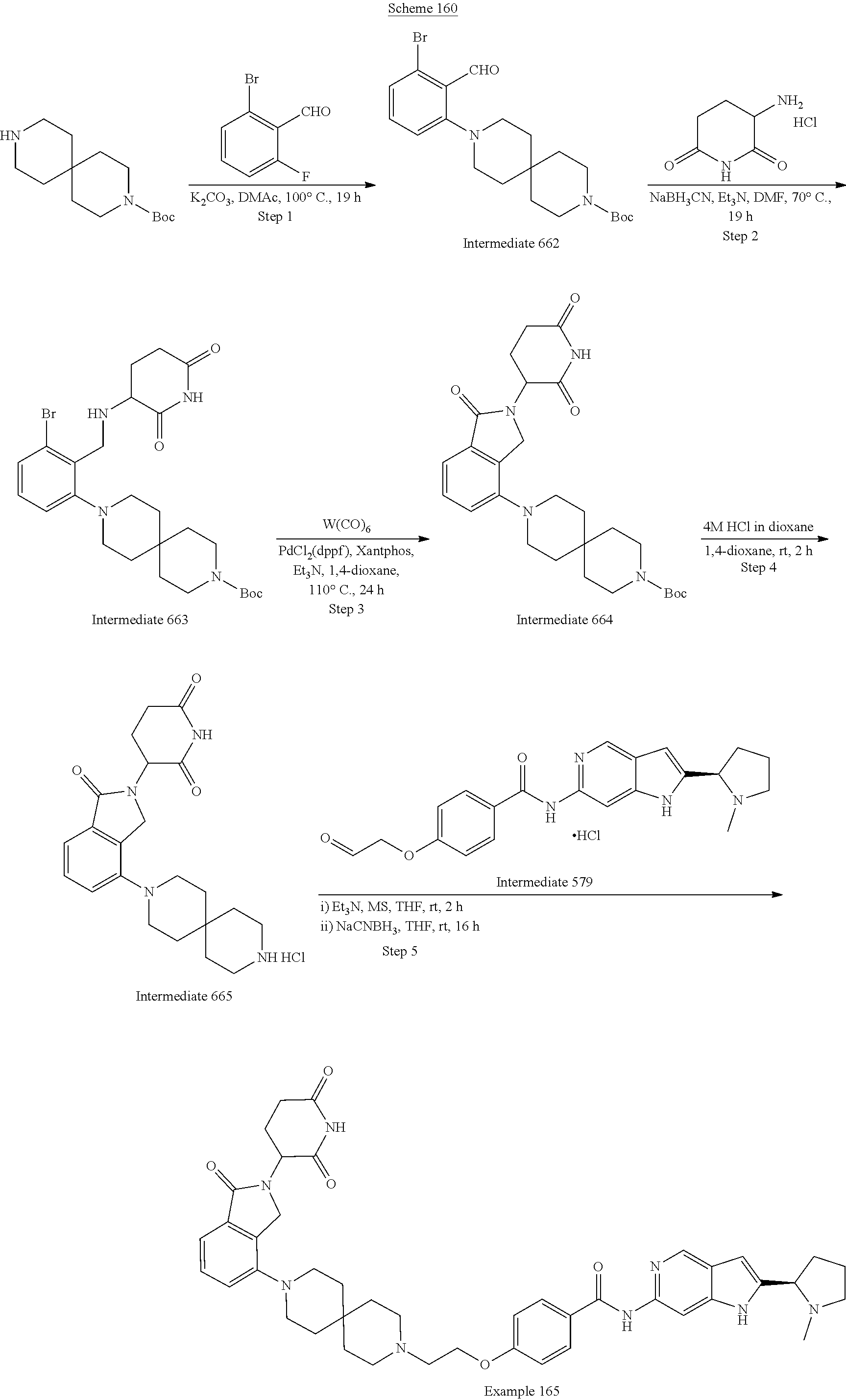

### Step 1

### Intermediate 662: tert-butyl 9-(3-bromo-2-formylphenyl)-3,9-diazaspiro[5.5]undecane-3-carboxylate

To a suspension of tert-butyl 3,9-diazaspiro[5.5]undecane-3-carboxylate (CAS No. 173405-78-2, 7.51 g, 29.6 mmol) in N,N-dimethylacetamide (20 mL) was added 2-bromo-6-fluorobenzaldehyde (CAS No. 360575-28-6, 5.0 g, 24.7 mmol) and potassium carbonate (8.51 g, 61.6 mmol) and the reaction mixture was heated at 100° C. for 16 h. The reaction mixture was quenched with ice cold water (200 mL) and extracted with ethyl acetate (2×50 mL). The organic layer was separated, dried over anhydrous Na₂SO₄, filtered and concentrated in vacuo to afford tert-butyl 9-(3-bromo-2-formylphenyl)-3,9-diazaspiro[5.5]undecane-3-carboxylate (Intermediate 662, 11.0 g) as an off-white solid, which was for the next step without further purification. ¹H NMR (400 MHz, DMSO-d₆) δ ppm 1.29-1.36 (m, 4H), 1.38 (s, 9H), 1.40-1.44 (m, 4H), 2.94-3.02 (m, 4H), 3.21-3.26 (m, 4H), 7.22 (d, J=7.83 Hz, 1H), 7.28 (d, J=7.83 Hz, 1H), 7.36-7.43 (m, 1H), 10.02 (br s, 1H). Mass spec m/z 439.1 [M+H+2]⁺.

### Step 2

### Intermediate 663: tert-butyl 9-(3-bromo-2-(((2,6-dioxopiperidin-3-yl)amino)methyl)phenyl)-3,9-diazaspiro[5.5]undecane-3-carboxylate

To a solution of tert-butyl 9-(3-bromo-2-formylphenyl)-3,9-diazaspiro[5.5]undecane-3-carboxylate (Intermediate 662, 11.0 g, 25.2 mmol) in dimethylformamide (20 mL) was added 3-aminopiperidine-2,6-dione hydrochloride (CAS No. 24666-56-6, 5.12 g, 30.2 mmol) followed by triethylamine (10.6 mL, 75.5 mmol) and the reaction was heated at 70° C. for 3 h. Sodium cyanoborohydride (6.65 g, 101 mmol) was then added and the reaction was heated at 70° C. for 16 h. The reaction mixture was diluted with water (50 mL), the resultant precipitate was collected by filtration and dried in vacuo to afford tert-butyl 9-(3-bromo-2-(((2,6-dioxopiperidin-3-yl)amino)methyl)phenyl)-3,9-diazaspiro[5.5]undecane-3-carboxylate (Intermediate 663, 10.0 g, 72%) as an off-white solid, which was used for the next step without further purification. ¹H NMR (401 MHz, DMSO-d₆) δ ppm 1.29-1.36 (m, 4H), 1.39 (s, 9H), 1.40-1.48 (m, 4H), 1.58-1.69 (m, 4H), 2.72-2.90 (m, 7H), 2.93-3.04 (m, 3H), 3.87-3.93 (m, 2H), 7.17-7.21 (m, 2H), 7.31-7.35 (m, 1H), 10.78 (br s, 1H). Mass spec m/z 551.2 [M+H]⁺.

### Step 3

### Intermediate 664: tert-butyl 9-(2-(2,6-dioxopiperidin-3-yl)-1-oxoisoindolin-4-yl)-3,9-diazaspiro[5.5]undecane-3-carboxylate

To a solution of tert-butyl 9-(3-bromo-2-(((2,6-dioxopiperidin-3-yl)amino)methyl)phenyl)-3,9-diazaspiro[5.5]undecane-3-carboxylate (Intermediate 663, 1.0 g, 1.82 mmol) in 1,4-dioxane (20 mL) was added triethylamine (1.27 mL, 9.09 mmol). The reaction mixture was purged with argon for 15 min, then tungsten hexacarbonyl (0.64 g, 1.82 mmol), PdCl₂(dppf) (0.13 g, 1.82 mmol) and Xantphos (0.32 g, 0.54 mmol) were added. The reaction mixture was further purged with argon for 10 min and heated at 110° C. for 24 h. The reaction mixture was filtered through a celite bed and the filter pad was washed with dichloromethane and the filtrate concentrated in vacuo. The crude material was purified by combi-flash chromatography, by eluting with 100% ethyl acetate, to afford tert-butyl 9-(2-(2,6-dioxopiperidin-3-yl)-1-oxoisoindolin-4-yl)-3,9-diazaspiro[5.5]undecane-3-carboxylate (Intermediate 664, 1.50 g) as a yellow solid which was used for the next step without further purification. Mass spec m/z 497.3 [M+H]⁺.

### Step 4

### Intermediate 665: 3-(1-oxo-4-(3,9-diazaspiro[5.5]undecan-3-yl)isoindolin-2-yl)piperidine-2,6-dione hydrochloride

To a cooled (0° C.) solution of tert-butyl 9-(2-(2,6-dioxopiperidin-3-yl)-1-oxoisoindolin-4-yl)-3,9-diazaspiro[5.5]undecane-3-carboxylate (Intermediate 664, 0.70 g, 1.0 mmol) in dichloromethane (7 mL) was added 4M hydrochloric acid in 1,4-dioxane (7 mL, 28 mmol) and the reaction mixture was stirred at room temperature for 2 h. The reaction mixture was concentrated in vacuo, the residue triturated with diethyl ether (2×10 mL) and dried in vacuo to afford 3-(1-oxo-4-(3,9-diazaspiro[5.5]undecan-3-yl)isoindolin-2-yl)piperidine-2,6-dione hydrochloride (Intermediate 665, 0.7 g, 70%) as a white solid, which was for the next step without further purification. ¹H NMR (400 MHz, DMSO-d₆) δ ppm 1.59-1.69 (m, 8H), 3.01-3.10 (m, 8H), 3.52-3.58 (m, 5H), 4.27-4.33 (m, 1H), 4.41-4.49 (m, 1H), 7.20-7.23 (m, 1H), 7.32 (d, J=7.13 Hz, 1H), 7.43-7.47 (m, 1H), 10.98 (br s, 1H). Mass spec m/z 397.1 [M+H]⁺.

### Step 5

### Example 165: 4-(2-(9-(2-(2,6-dioxopiperidin-3-yl)-1-oxoisoindolin-4-yl)-3,9-diazaspiro[5.5]undecan-3-yl)ethoxy)-N-(2-((R)-1-methylpyrrolidin-2-yl)-1H-pyrrolo[3,2-c]pyridin-6-yl)benzamide

To a solution of (R)—N-(2-(1-methylpyrrolidin-2-yl)-1H-pyrrolo[3,2-c]pyridin-6-yl)-4-(2-oxoethoxy)benzamide hydrochloride (Intermediate 579, 0.4 g, 0.96 mmol) in tetrahydrofuran (5 mL) was added 3-(1-oxo-4-(3,9-diazaspiro[5.5]undecan-3-yl)isoindolin-2-yl)piperidine-2,6-dione hydrochloride (Intermediate 665, 0.41 g, 0.96 mmol) and triethylamine (0.40 mL 2.89 mmol) and the reaction mixture was stirred at room temperature for 2 h. Sodium cyanoborohydride (0.12 g, 1.92 mmol) was added and the reaction mixture was stirred at room temperature for 16 h. The reaction mixture was diluted with water (50 mL), the resultant precipitate was collected by filtration and dried in vacuo. The crude material was purified by preparative HPLC (Method B) to afford 4-(2-(9-(2-(2,6-dioxopiperidin-3-yl)-1-oxoisoindolin-4-yl)-3,9-diazaspiro[5.5]undecan-3-yl)ethoxy)-N-(2-((R)-1-methylpyrrolidin-2-yl)-1H-pyrrolo[3,2-c]pyridin-6-yl)benzamide (Example 165, 0.027 g, 4%) as an off-white solid. ¹H NMR (400 MHz, DMSO-d₆) δ ppm 1.50-1.60 (m, 8H), 1.76-2.00 (m, 4H), 2.11-2.15 (m, 1H), 2.16 (s, 3H), 2.22-2.30 (m, 1H), 2.43-2.47 (m, 2H), 2.52-2.63 (m, 4H), 2.70-2.78 (m, 2H), 2.86-2.97 (m, 1H), 2.99-3.07 (m, 4H), 3.11-3.17 (m, 1H), 3.26-3.31 (m, 1H), 4.14-4.17 (m, 2H), 4.25-4.32 (m, 1H), 4.39-4.45 (m, 1H), 5.08-5.13 (m, 1H), 6.38 (s, 1H), 7.03 (d, J=8.88 Hz, 2H), 7.18 (d, J=7.63 Hz, 1H), 7.28 (d, J=7.25 Hz, 1H), 7.40-7.45 (m, 1H), 8.04 (d, J=8.76 Hz, 2H), 8.17 (s, 1H), 8.48 (s, 1H), 10.28 (br s, 1H), 10.98 (br s, 1H), 11.34 (br s, 1H). Mass spec m/z 759.3 [M+H]⁺.

### Example 166 was Synthesised Following Scheme 161

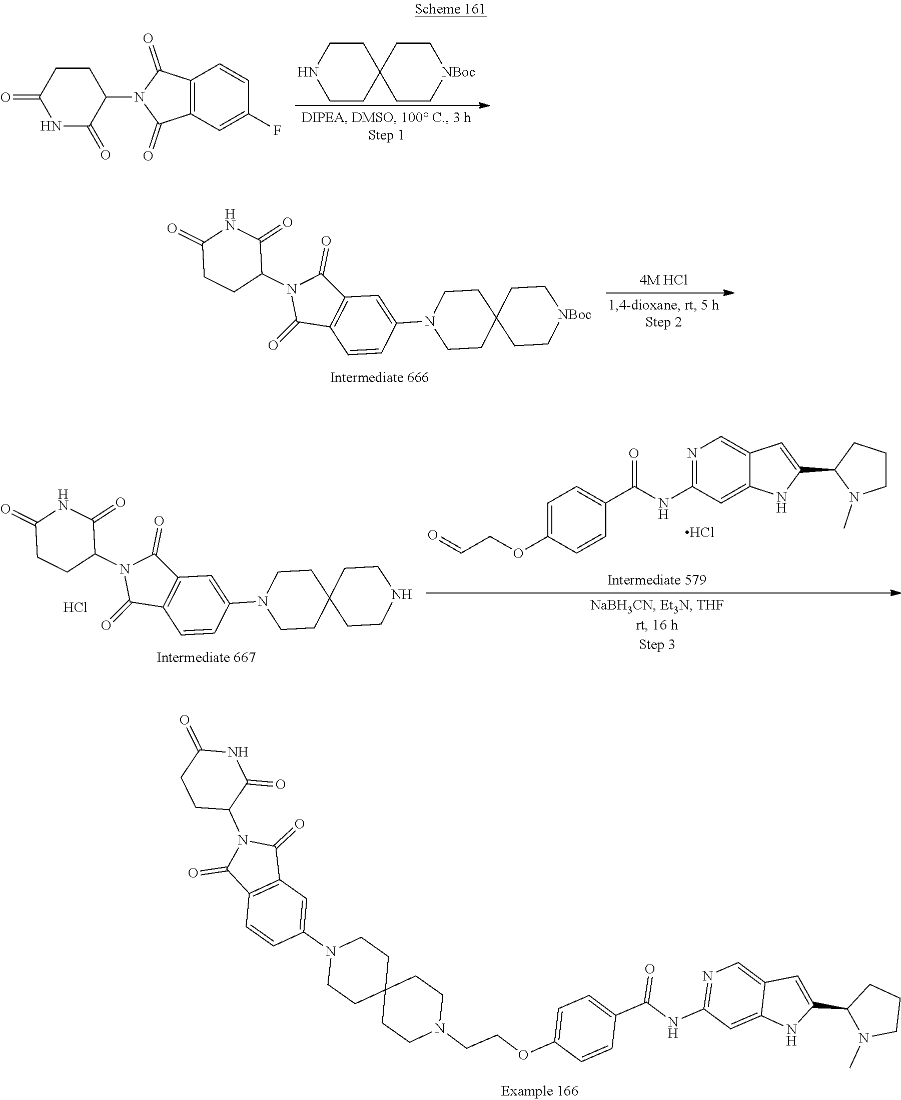

### Step 1

### Intermediate 666: tert-butyl 9-(2-(2,6-dioxopiperidin-3-yl)-1,3-dioxoisoindolin-5-yl)-3,9-diazaspiro[5.5]undecane-3-carboxylate

To a stirred solution of 2-(2,6-dioxopiperidin-3-yl)-5-fluoroisoindoline-1,3-dione (CAS No. 835616-61-0, 2.0 g, 7.24 mmol) in dimethyl sulfoxide (20 mL) was added tert-butyl 3,9-diazaspiro[5.5]undecane-3-carboxylate (CAS No. 173405-78-2, 1.8 g, 7.24 mmol) and N,N-diisopropylethylamine (5.06 mL, 28.9 mmol) and the reaction mixture was stirred at 1000° C. for 3 h. The reaction mixture was diluted with ice water (200 mL) and extracted with ethyl acetate (3×100 mL). The combined organic layers were dried over anhydrous Na₂SO₄ and concentrated in vacuo. The crude material was purified by combi-flash chromatography, by eluting with ethyl acetate, to afford tert-butyl 9-(2-(2,6-dioxopiperidin-3-yl)-1,3-dioxoisoindolin-5-yl)-3,9-diazaspiro[5.5]undecane-3-carboxylate (Intermediate 666, 1.9 g, 51%) as yellow solid. ¹H NMR (400 MHz, DMSO-d₆) δ ppm 1.35-1.39 (m, 14H), 1.50-1.60 (m, 4H), 1.95-2.03 (m, 1H), 2.52-2.59 (m, 4H), 2.80-2.89 (m, 2H), 3.43-3.49 (m, 4H), 5.01-5.08 (m, 1H), 7.21 (d, J=7.60 Hz, 1H), 7.29 (s, 1H), 7.63 (d, J=8.4 Hz, 1H), 11.06 (s, 1H). Mass spec m/z 511.3 [M+H]⁺.

### Step 2

### Intermediate 667: 2-(2,6-dioxopiperidin-3-yl)-5-(3,9-diazaspiro[5.5]undecan-3-yl)isoindoline-1,3-dione hydrochloride

To a cooled (0° C.) solution of tert-butyl 9-(2-(2,6-dioxopiperidin-3-yl)-1,3-dioxoisoindolin-5-yl)-3,9-diazaspiro[5.5]undecane-3-carboxylate (Intermediate 666, 0.5 g, 0.98 mmol) in 1,4-dioxane (5 mL) was added 4M HCl in 1,4-dioxane (5 mL) and the reaction mixture was stirred at room temperature for 16 h. The reaction mixture was concentrated in vacuo to afford 2-(2,6-dioxopiperidin-3-yl)-5-(3,9-diazaspiro[5.5]undecan-3-yl)isoindoline-1,3-dione hydrochloride (Intermediate 667, 0.4 g) as an off-white solid, which was used for next step without further purification. ¹H NMR (400 MHz, DMSO-d₆) δ ppm 1,22-1.25 (m, 1H), 1.55-1.59 (m, 4H), 1.64-1.68 (m, 4H), 1.97-2.03 (m, 1H), 2.81-2.91 (m, 1H), 2.98-3.05 (m, 5H), 3.45-3.40 (m, 4H), 5.03-5.09 (m, 1H), 7.24 (d, J=8.00 Hz, 1H), 7.32 (s, 1H), 7.66 (d, J=8.00 Hz, 1H), 8.82-8.9 (m, 2H), 11.07 (s, 1H). Mass spec m/z 409.1 [M+H]⁺.

### Step 3

### Example 166: 4-(2-(9-(2-(2,6-dioxopiperidin-3-yl)-1,3-dioxoisoindolin-5-yl)-3,9-diazaspiro[5.5]undecan-3-yl)ethoxy)-N-(2-((R)-1-methylpyrrolidin-2-yl)-1H-pyrrolo[3,2-c]pyridin-6-yl)benzamide

To a solution of (R)—N-(2-(1-methylpyrrolidin-2-yl)-1H-pyrrolo[3,2-c]pyridin-6-yl)-4-(2-oxoethoxy)benzamide hydrochloride (Intermediate 579, 0.4 g, 1.04 mmol) in tetrahydrofuran (10.0 mL) was added triethylamine (0.74 mL, 5.28 mmol) followed by 2-(2,6-dioxopiperidin-3-yl)-5-(3,9-diazaspiro[5.5]undecan-3-yl)isoindoline-1,3-dione hydrochloride (Intermediate 667, 0.43 g, 0.96 mmol) and the reaction mixture was stirred at room temperature for 5 h. Sodium cyanoborohydride (0.19 g 2.89 mmol) was added and the reaction mixture was stirred at room temperature for 16 h, then quenched with ice cold water (100 mL). The resultant precipitate was collected by filtration and dried in vacuo. The crude material was purified was purified by preparative HPLC (Method A) to afford 4-(2-(9-(2-(2,6-dioxopiperidin-3-yl)-1,3-dioxoisoindolin-5-yl)-3,9-diazaspiro[5.5]undecan-3-yl)ethoxy)-N-(2-((R)-1-methylpyrrolidin-2-yl)-1H-pyrrolo[3,2-c]pyridin-6-yl)benzamide (Example 166, 0.09 g, 12%) as an off-white solid. ¹H NMR (400 MHz, DMSO-d₆) δ ppm 1.48-1.53 (m, 8H), 1.76-1.93 (m, 3H), 1.95-2.04 (m, 1H), 2.14-2.19 (m, 4H), 2.22-2.29 (m, 1H), 2.41-2.48 (m, 2H), 2.53-2.62 (m, 4H), 2.71-2.75 (m, 2H), 2.83-2.92 (m, 1H), 3.12-3.18 (m, 1H), 3.27-3.29 (m, 1H), 3.47-3.49 (m, 4H), 4.15-4.18 (m, 2H), 5.04-5.08 (m, 1H), 6.38 (s, 1H), 7.03 (d, J=8.80 Hz, 2H), 7.20-7.23 (m, 1H), 7.29-7.31 (m, 1H), 7.64 (d, J=8.80 Hz, 1H), 8.03 (d, J=8.80 Hz, 2H), 8.17 (s, 1H), 8.49 (s, 1H), 10.29 (s, 1H), 11.18 (s, 1H), 11.34 (s, 1H). Mass spec m/z 773.5 [M+H]⁺.

### Example 167 was Synthesised Following Scheme 162

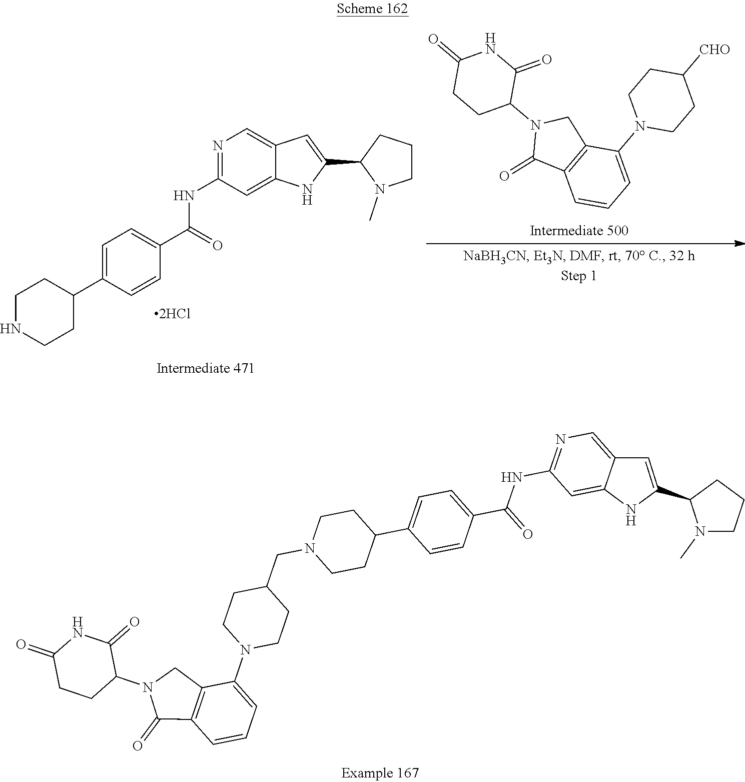 [SMILES: CN1CCC[C@@H]1c1cc2cnc(NC(=O)c3ccc(C4CCNCC4)cc3)cc2[nH]1 ; O=CC1CCN(c2cccc3c2CN(C2CCC(=O)NC2=O)C3=O)CC1 ; CN1CCC[C@@H]1c1cc2cnc(NC(=O)c3ccc(C4CCN(CC5CCN(c6cccc7c6CN(C6CCC(=O)NC6=O)C7=O)CC5)CC4)cc3)cc2[nH]1]

### Step 1

### Example 167: 4-(1-((1-(2-(2,6-dioxopiperidin-3-yl)-1-oxoisoindolin-4-yl)piperidin-4-yl)methyl)piperidin-4-yl)-N-(2-((R)-1-methylpyrrolidin-2-yl)-1H-pyrrolo[3,2-c]pyridin-6-yl)benzamide

To a solution of (R)—N-(2-(1-methylpyrrolidin-2-yl)-1H-pyrrolo[3,2-c]pyridin-6-yl)-4-(piperidin-4-yl)benzamide dihydrochloride (Intermediate 471, 0.65 g, 4.64 mmol) in dimethylformamide (10 mL) were added 1-(2-(2,6-dioxopiperidin-3-yl)-1-oxoisoindolin-4-yl)piperidine-4-carbaldehyde (Intermediate 500, 0.55 g, 1.55 mmol) and triethylamine (0.65 mL, 4.64 mmol) and the reaction mixture was heated at 70° C. for 16 h. Sodium cyanoborohydride (0.46 g, 6.19 mmol) was then added and the reaction mixture heated at 70° C. for 16 h. The reaction mixture was then quenched with ice cold water (50 mL), the resultant precipitate was collected by filtration and dried in vacuo. The crude material was purified by preparative HPLC (Method B) to afford 4-(1-((1-(2-(2,6-dioxopiperidin-3-yl)-1-oxoisoindolin-4-yl)piperidin-4-yl)methyl)piperidin-4-yl)-N-(2-((R)-1-methylpyrrolidin-2-yl)-1H-pyrrolo[3,2-c]pyridin-6-yl)benzamide (Example 167, 0.02 g, 2%) as an off-white solid. ¹H NMR (400 MHz, DMSO-d₆) δ ppm 1.21-1.31 (m, 2H), 1.65-1.89 (m, 10H), 1.97-2.06 (m, 3H), 2.14-2.17 (m, 4H), 2.22-2.26 (m, 3H), 2.54-2.61 (m, 2H), 2.71-2.79 (m, 2H), 2.89-3.01 (m, 3H), 3.12-3.16 (m, 1H), 3.25-3.36 (m, 6H), 4.27-4.31 (m, 1H), 4.41-4.46 (m, 1H), 5.09-5.15 (m, 1H), 6.39 (s, 1H), 7.16 (d, J=7.6 Hz, 1H) 7.29 (d, J=7.2 Hz, 1H), 7.37 (d, J=8.4 Hz, 2H), 7.42 (d, J=7.6 Hz, 1H), 7.98 (d, J=8.4 Hz, 2H) 8.18-8.20 (m, 2H), 8.49 (s, 1H). Mass spec m/z 743.2 [M+H]⁺.

### Example 168 was Synthesised Following Scheme 163

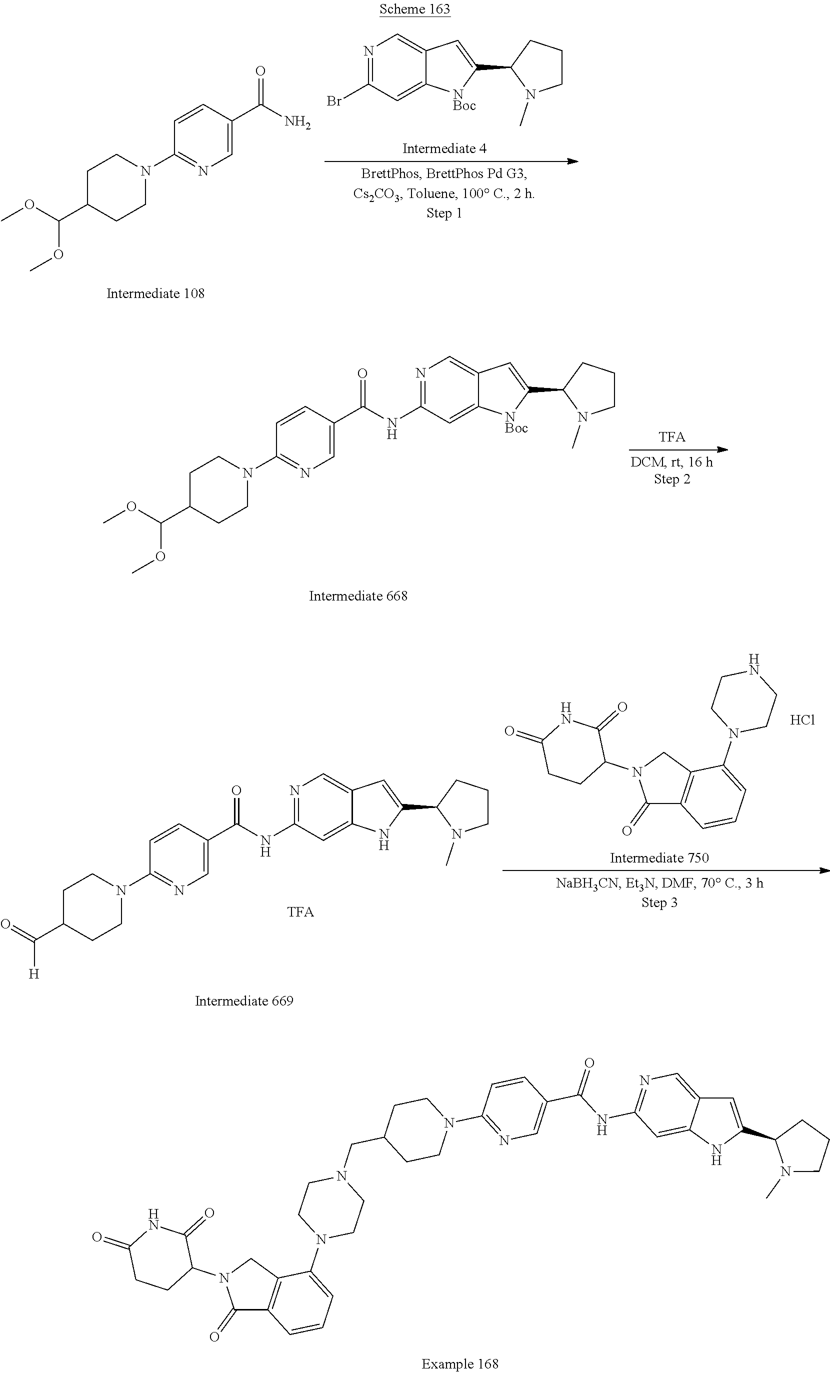

### Step 1

### Intermediate 668: tert-butyl (R)-6-(6-(4-(dimethoxymethyl)piperidin-1-yl)nicotinamido)-2-(1-methylpyrrolidin-2-yl)-1H-pyrrolo[3,2-c]pyridine-1-carboxylate

To a solution of 6-(4-(dimethoxymethyl)piperidin-1-yl)nicotinamide (Intermediate 108, 0.58 g, 2.10 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 1.0 g, 2.63 mmol), followed by cesium carbonate (2.57 g, 7.88 mmol). The reaction mixture was purged with argon for 15 min, then BrettPhos Pd G3 (0.36 g, 0.39 mmol) and BrettPhos (0.21 g, 0.39 mmol) were added. The reaction mixture was further purged with argon for 10 min and heated at 100° C. for 2 h then concentrated in vacuo. The crude material was purified by combi-flash chromatography, by eluting with 70% ethyl acetate in heptane, to afford tert-butyl (R)-6-(6-(4-(dimethoxymethyl)piperidin-1-yl)nicotinamido)-2-(1-methylpyrrolidin-2-yl)-1H-pyrrolo[3,2-c]pyridine-1-carboxylate (Intermediate 668, 0.6 g, 24%) as an off-white solid. Mass spec m/z 579.1 [M+H]⁺.

### Step 2

### Intermediate 669: (R)-6-(4-formylpiperidin-1-yl)-N-(2-(1-methylpyrrolidin-2-yl)-1H-pyrrolo[3,2-c]pyridin-6-yl)nicotinamide trifluoroacetate

To a cooled (0° C.) solution of tert-butyl (R)-6-(6-(4-(dimethoxymethyl)piperidin-1-yl) nicotinamide)-2-(1-methylpyrrolidin-2-yl)-1H-pyrrolo[3,2-c]pyridine-1-carboxylate (Intermediate 668, 0.50 g, 0.86 mmol) in dichloromethane (5 mL) was added trifluoroacetic acid (1.00 mL, 13.0 mmol) and the reaction mixture was stirred at room temperature for 16 h. The reaction mixture was concentrated in vacuo. The crude material was triturated with diethyl ether (10 mL) and dried in vacuo to afford (R)-6-(4-formylpiperidin-1-yl)-N-(2-(1-methylpyrrolidin-2-yl)-1H-pyrrolo[3,2-c]pyridin-6-yl)nicotinamide trifluoroacetate (Intermediate 669, 0.55 g, 63%) as an off-white solid, which was used for the next step without further purification. Mass spec m/z 433.3 [M+H]⁺.

### Step 4

### Example 168: 6-(4-((4-(2-(2,6-dioxopiperidin-3-yl)-1-oxoisoindolin-4-yl)piperazin-1-yl)methyl)piperidin-1-yl)-N-(2-((R)-1-methylpyrrolidin-2-yl)-1H-pyrrolo[3,2-c]pyridin-6-yl)nicotinamide

To a solution of (R)-6-(4-formylpiperidin-1-yl)-N-(2-(1-methylpyrrolidin-2-yl)-1H-pyrrolo[3,2-c]pyridin-6-yl)nicotinamide trifluoroacetate (Intermediate 669, 0.55 g, 1.27 mmol) in dimethylformamide (2 mL) was added triethylamine (0.71 mL, 5.08 mmol) followed by 3-(1-oxo-4-(piperazin-1-yl)isoindolin-2-yl)piperidine-2,6-dione (Intermediate 750, 0.41 g, 1.27 mmol) and the reaction mixture was heated at 70° C. for 1 h. Sodium cyanoborohydride (0.25 g 3.81 mmol) was then added and the reaction mixture heated at 70° C. for 2 h. The reaction mixture was then diluted with ice cold water (100 mL) and extracted with ethyl acetate (2×100 mL). The combined organic layers were dried over Na₂SO₄, filtered, and concentrated in vacuo The crude material was purified by preparative HPLC (Method A) to afford 6-(4-((4-(2-(2,6-dioxopiperidin-3-yl)-1-oxoisoindolin-4-yl)piperazin-1-yl)methyl)piperidin-1-yl)-N-(2-((R)-1-methylpyrrolidin-2-yl)-1H-pyrrolo[3,2-c]pyridin-6-yl)nicotinamide (Example 168, 0.03 g, 3%) as an off-white solid. ¹H NMR (400 MHz, DMSO-d₆) δ ppm 1.06-1.14 (m, 2H), 1.80-1.95 (m, 6H), 1.95-2.03 (m, 1H), 2.16 (s, 3H), 2.17-2.19 (m, 1H), 2.20-2.24 (m, 3H), 2.34-2.40 (m, 2H), 2.52-2.55 (m, 2H), 2.60-2.65 (m, 1H), 2.87-2.95 (m, 3H), 3.02-3.17 (m, 6H), 3.25-3.29 (m, 1H), 4.28-4.32 (m, 1H), 4.43-4.47 (m, 3H), 5.09-5.13 (m, 1H), 6.37 (s, 1H), 6.86 (d, J=9.13 Hz, 1H), 7.17 (d, J=7.50 Hz, 1H), 7.32 (d, J=7.50 Hz, 1H), 7.44 (t, J=7.63 Hz, 1H), 8.12-8.16 (m, 2H), 8.47 (s, 1H), 8.78 (s, 1H), 10.23 (br s, 1H), 10.98 (br s, 1H), 11.33 (br s, 1H). Mass spec m/z 745.3 [M+H]⁺.

### Example 169 was Synthesised Following Scheme 164

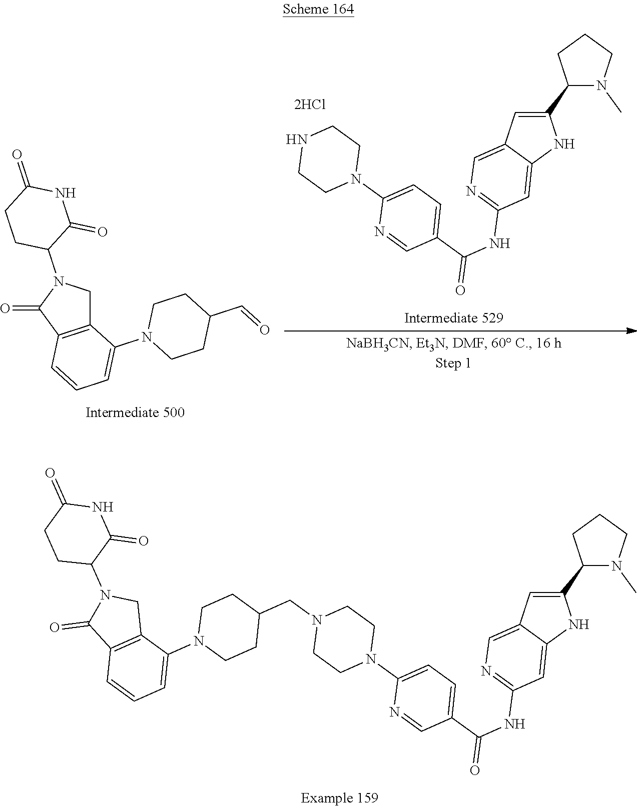 [SMILES: O=CC1CCN(c2cccc3c2CN(C2CCC(=O)NC2=O)C3=O)CC1 ; CN1CCC[C@@H]1c1cc2cnc(NC(=O)c3ccc(N4CCNCC4)nc3)cc2[nH]1 ; CN1CCC[C@@H]1c1cc2cnc(NC(=O)c3ccc(N4CCN(CC5CCN(c6cccc7c6CN(C6CCC(=O)NC6=O)C7=O)CC5)CC4)nc3)cc2[nH]1]

### Step 1

### Example 169: 6-(4-((1-(2-(2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)nicotinamide

To a solution of 1-(2-(2,6-dioxopiperidin-3-yl)-1-oxoisoindolin-4-yl)piperidine-4-carbaldehyde (Intermediate 500, 0.45 g, 0.95 mmol) and (R)—N-(2-(1-methylpyrrolidin-2-yl)-1H-pyrrolo[3,2-c]pyridin-6-yl)-6-(piperazin-1-yl)nicotinamide dihydrochloride (Intermediate 529, 0.46 g, 1.15 mmol) in dimethylformamide (30 mL) was added triethylamine (0.67 mL, 4.79 mmol) and sodium cyanoborohydride (0.19 g, 2.87 mmol) and the reaction mixture was heated at 60° C. for 16 h. The reaction mixture was concentrated in vacuo, diluted with ice cold water (50 mL) and the resultant precipitate was collected by filtration and dried in vacuo. The crude material was purified by preparative HPLC (Method B) to afford 6-(4-((1-(2-(2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)nicotinamide (Example 169, 0.02 g, 3%) as an off-white solid. ¹H NMR (400 MHz, DMSO-d₆) δ ppm 1.23-1.30 (m, 2H), 1.71-1.81 (m, 2H), 1.82-1.91 (m, 4H), 1.97-2.02 (m, 1H), 2.10-2.17 (m, 1H), 2.16 (s, 3H), 2.23-2.28 (m, 3H), 2.42-2.45 (m, 5H), 2.61-2.68 (m, 2H), 2.72-2.81 (m, 2H), 2.87-2.94 (m, 1H), 3.11-3.17 (m, 1H), 3.38-3.44 (m, 2H), 3.60-3.65 (m, 4H), 4.26-4.31 (m, 1H), 4.41-4.45 (m, 1H), 5.09-5.13 (m, 1H), 6.37 (s, 1H), 6.87 (d, J=9.13 Hz, 1H), 7.17 (d, J=7.63 Hz, 1H), 7.29 (d, J=7.25 Hz, 1H), 7.40-7.46 (m, 1H), 8.14-8.19 (m, 2H), 8.48 (s, 1H), 8.80 (s, 1H), 10.27 (br s, 1H), 10.97 (br s, 1H), 11.32 (br s, 1H). Mass spec m/z 745.1 [M+H]⁺.

### Example 170 was Synthesised Following Scheme 165

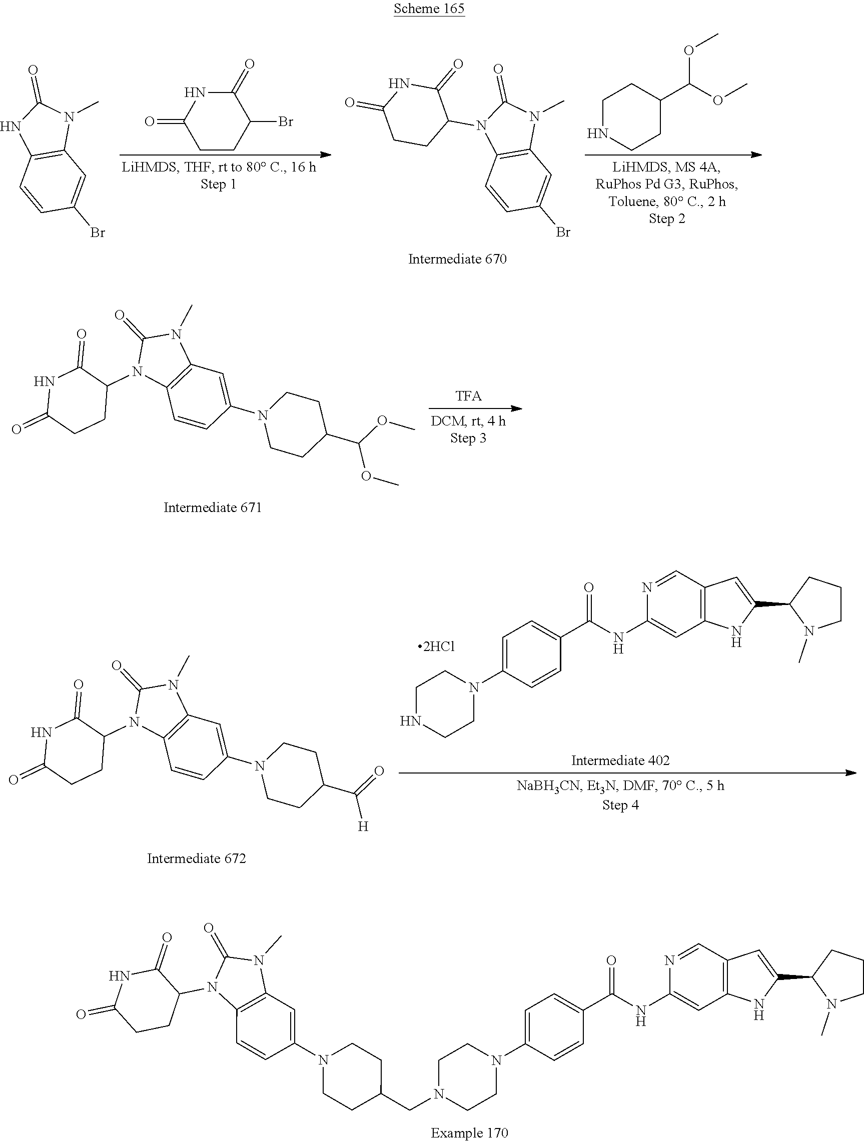

### Step 1

### Intermediate 670: 3-(5-bromo-3-methyl-2-oxo-2,3-dihydro-1H-benzo[d]imidazol-1-yl)piperidine-2,6-dione

To a solution of 6-bromo-1-methyl-1,3-dihydro-2H-benzo[d]imidazol-2-one (CAS No. 305790-48-1, 2.0 g, 8.80 mmol) and 3-bromopiperidine-2,6-dione (CAS No. 62595-74-8, 8.45 g, 44.0 mmol) in tetrahydrofuran (50 mL) was added LiHMDS (1.5 M in THF, 14.68 mL, 22.0 mmol) and the reaction mixture was heated at 60° C. for 16 h. The reaction mixture was then concentrated in vacuo, diluted with ethyl acetate (200 mL) and washed with ammonium chloride solution (100 mL). The organic layer was separated, dried over anhydrous Na₂SO₄, filtered and concentrated in vacuo. The crude material was purified by combi-flash chromatography, by eluting with 70% ethyl acetate in heptane, to afford 3-(5-bromo-3-methyl-2-oxo-2,3-dihydro-1H-benzo[d]imidazol-1-yl)piperidine-2,6-dione (Intermediate 670, 1.1 g, 37%) as a brown solid. ¹H NMR (401 MHz, DMSO-d₆) δ ppm 2.59-2.74 (m, 4H), 3.26 (s, 3H), 5.34-5.42 (m, 1H), 7.21 (dd, J=8.31, 1.83 Hz, 1H), 7.31-7.35 (m, 1H), 7.47 (s, 1H), 11.13 (br s, 1H). Mass spec m/z 339.9 [M+H+2]⁺.

### Step 2

### Intermediate 671: 3-(5-(4-(dimethoxymethyl)piperidin-1-yl)-3-methyl-2-oxo-2,3-dihydro-1H-benzo[d]imidazol-1-yl)piperidine-2,6-dione

To a solution of 3-(5-bromo-3-methyl-2-oxo-2,3-dihydro-1H-benzo[d]imidazol-1-yl)piperidine-2,6-dione (Intermediate 670, 1.10 g, 3.3 mmol) and 4-(dimethoxymethyl)piperidine (CAS No. 188646-83-5, 0.57 g, 3.6 mmol) in toluene (20 mL) was added LiHMDS (1.5 M in THF, 8.7 mL, 13.0 mmol. RuPhos (0.46 g, 0.98 mmol), RuPhos Pd G3 (0.43 g, 0.49 mmol) were then added, and the reaction mixture was heated at 80° C. for 2 h. The reaction mixture was filtered through a celite bed and the filtrate concentrated in vacuo. The crude material was purified by combi-flash chromatography, by eluting with 5% MeOH in DCM, to afford 3-(5-(4-(dimethoxymethyl)piperidin-1-yl)-3-methyl-2-oxo-2,3-dihydro-1H-benzo[d]imidazol-1-yl)piperidine-2,6-dione (Intermediate 671, 0.7 g, 50%) as yellow solid. ¹H NMR (400 MHz, DMSO-d₆) δ ppm 1.31-1.42 (m, 4H), 1.65-1.73 (m, 4H), 2.51-2.60 (m, 4H), 3.22 (s, 3H), 3.28 (s, 6H), 3.51-3.63 (m, 2H), 4.05-4.11 (m, 1H), 6.54-6.65 (m, 1H), 6.71 (s, 1H), 6.80 (d, J=8.69 Hz, 1H), 10.46 (br s, 1H). Mass spec m/z 417.1 [M+H]⁺.

### Step 3

### Intermediate 672: 1-(1-(2,6-dioxopiperidin-3-yl)-3-methyl-2-oxo-2,3-dihydro-1H-benzo[d]imidazol-5-yl)piperidine-4-carbaldehyde

To a cooled (0° C.) solution of 3-(5-(4-(dimethoxymethyl)piperidin-1-yl)-3-methyl-2-oxo-2,3-dihydro-1H-benzo[d]imidazol-1-yl)piperidine-2,6-dione (Intermediate 671, 0.7 g, 2.0 mmol) in dichloromethane (14 mL) was added trifluoroacetic acid (3.0 g, 30.0 mmol) and the reaction mixture was stirred at room temperature for 4 h. The reaction mixture was concentrated in vacuo to afford 1-(1-(2,6-dioxopiperidin-3-yl)-3-methyl-2-oxo-2,3-dihydro-1H-benzo[d]imidazol-5-yl)piperidine-4-carbaldehyde (Intermediate 672, 0.7 g, 90%) as brown gummy solid which was used for next step without further purification. ¹H NMR (400 MHz, DMSO-d₆) δ ppm 1.06-1.34 (m, 2H), 1.62-2.05 (m, 4H), 2.07-2.23 (m, 2H), 2.58-2.75 (m, 2H), 3.28 (s, 3H), 3.43-3.64 (m, 3H), 5.29-5.43 (m, 1H), 6.95 (s, 1H), 7.00-7.41 (m, 2H), 9.67 (br s, 1H), 11.09 (br s, 1H). Mass spec m/z 371.0 [M+H]⁺.

### Step 4

### Example 170: 4-(4-((1-(1-(2,6-dioxopiperidin-3-yl)-3-methyl-2-oxo-2,3-dihydro-1H-benzo[d]imidazol-5-yl)piperidin-4-yl)methyl)piperazin-1-yl)-N-(2-((R)-1-methylpyrrolidin-2-yl)-1H-pyrrolo[3,2-c]pyridin-6-yl)benzamide

To a solution of (R)—N-(2-(1-methylpyrrolidin-2-yl)-1H-pyrrolo[3,2-c]pyridin-6-yl)-4-(piperazin-1-yl)benzamide (Intermediate 402, 0.5 g, 1.0 mmol) and 1-(1-(2,6-dioxopiperidin-3-yl)-3-methyl-2-oxo-2,3-dihydro-1H-benzo[d]imidazol-5-yl)piperidine-4-carbaldehyde (Intermediate 672, 0.5 g, 1.0 mmol) in dimethylformamide (5 mL) was added triethylamine (0.8 mL, 5.0 mmol) and the reaction mixture was heated at 70° C. for 3 h. Sodium cyanoborohydride (0.3 g, 4 mmol) was then added and the reaction mixture was heated at 70° C. for 2 h. The reaction mixture was diluted with water and the resultant precipitate was collected by filtration and dried in vacuo. The crude material was purified by preparative HPLC (Method A) to afford 4-(4-((1-(1-(2,6-dioxopiperidin-3-yl)-3-methyl-2-oxo-2,3-dihydro-1H-benzo[d]imidazol-5-yl)piperidin-4-yl)methyl)piperazin-1-yl)-N-(2-((R)-1-methylpyrrolidin-2-yl)-1H-pyrrolo[3,2-c]pyridin-6-yl)benzamide (Example 170, 0.040 g, 4%) as a white solid. ¹H NMR (400 MHz, DMSO-d₆) δ ppm 1.23-1.30 (m, 2H), 1.62-1.73 (m, 1H), 1.77-1.90 (m, 5H), 1.94-2.01 (m, 1H), 2.11-2.14 (m, 1H), 2.15 (s, 3H), 2.20-2.26 (m, 3H), 2.40-2.45 (m, 6H), 2.52-2.56 (m, 4H), 2.58-2.62 (m, 2H), 2.81-2.90 (m, 1H), 3.09-3.16 (m, 1H), 3.25-3.28 (m, 4H), 3.54-3.61 (m, 2H), 5.23-5.30 (m, 1H), 6.35 (s, 1H), 6.62 (dd, J=8.51, 1.50 Hz, 1H), 6.81 (s, 1H), 6.91 (d, J=8.50 Hz, 1H), 6.96 (d, J=8.88 Hz, 2H), 7.94 (d, J=9.01 Hz, 2H), 8.15 (s, 1H), 8.46 (s, 1H), 10.06 (br s, 1H), 11.03 (br s, 1H), 11.29 (br s, 1H). Mass spec m/z 759.3 [M+H]⁺.

### Example 171 was Synthesised Following Scheme 166

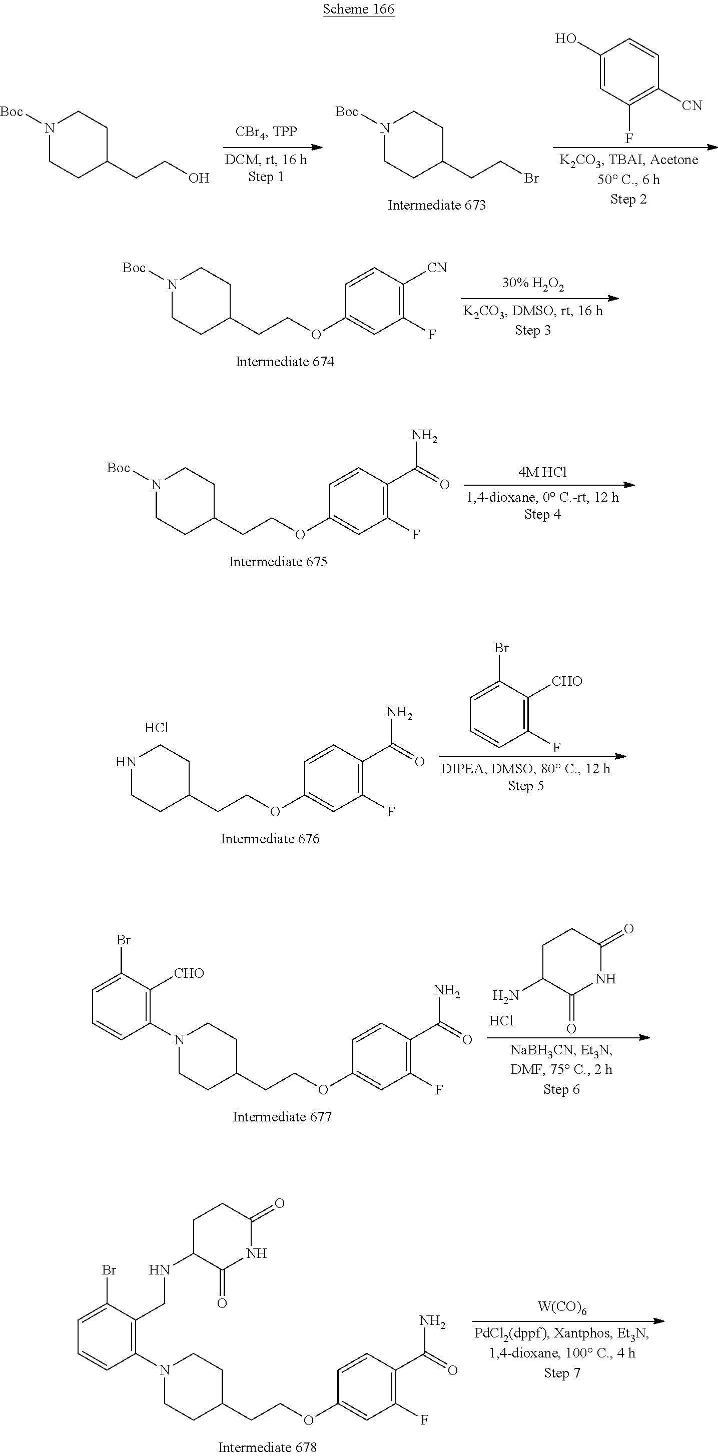

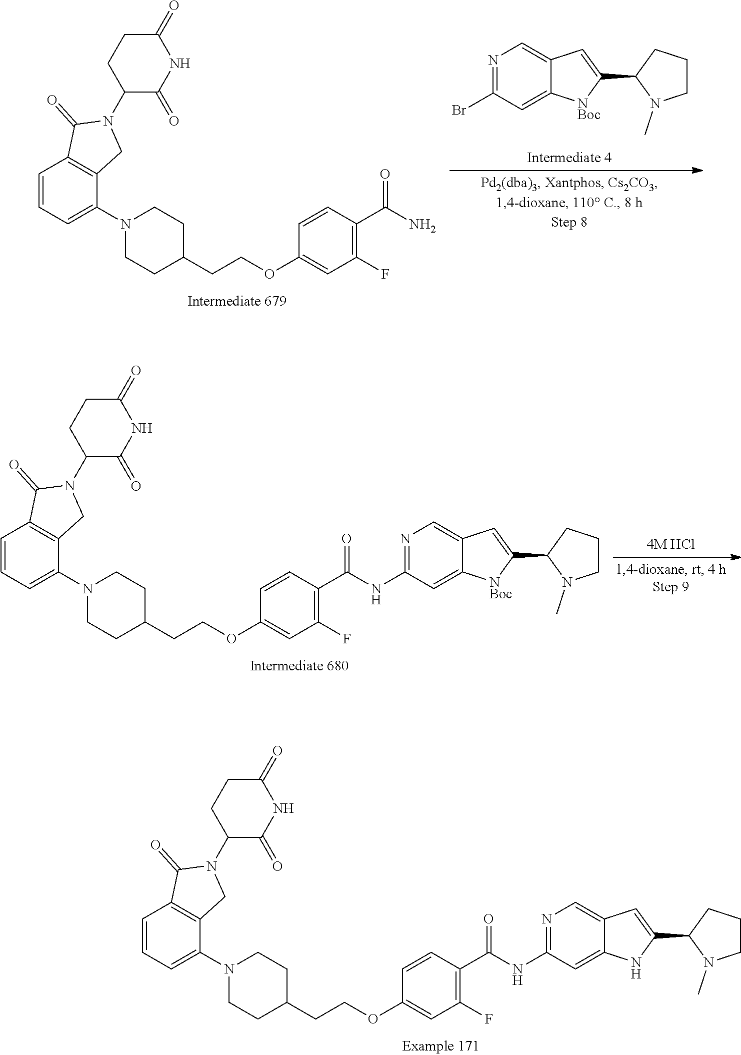 [SMILES: NC(=O)c1ccc(OCCC2CCN(c3cccc4c3CN(C3CCC(=O)NC3=O)C4=O)CC2)cc1F ; CN1CCC[C@@H]1c1cc2cnc(Br)cc2n1C(=O)OC(C)(C)C ; CN1CCC[C@@H]1c1cc2cnc(NC(=O)c3ccc(OCCC4CCN(c5cccc6c5CN(C5CCC(=O)NC5=O)C6=O)CC4)cc3F)cc2n1C(=O)OC(C)(C)C ; CN1CCC[C@@H]1c1cc2cnc(NC(=O)c3ccc(OCCC4CCN(c5cccc6c5CN(C5CCC(=O)NC5=O)C6=O)CC4)cc3F)cc2[nH]1]

### Step 1

### Intermediate 673: tert-butyl 4-(2-bromoethyl)piperidine-1-carboxylate

To a solution of tert-butyl 4-(2-hydroxyethyl)piperidine-1-carboxylate (CAS No. 77279-24-4, 50.0 g, 218 mmol) in dichloromethane (500 mL) was added triphenylphosphine (68.6 g, 262 mmol) followed by carbon tetrabromide (118 g, 349 mmol) and the reaction mixture was stirred at room temperature for 16 h. The reaction mixture was concentrated in vacuo and the crude material was purified by combi-flash chromatography, by eluting with 35% ethyl acetate in heptane, to afford tert-butyl 4-(2-bromoethyl)piperidine-1-carboxylate (Intermediate 673, 55 g, 86%) as colorless oil. ¹H NMR (400 MHz, DMSO-d₆) δ ppm 0.95-0.99 (m, 2H), 1.38 (s, 9H), 1.61-1.64 (m, 3H), 1.72-1.78 (m, 2H), 2.65-2.67 (m, 2H), 3.53-3.59 (m, 2H), 3.86-3.96 (m, 2H).

### Step 2

### Intermediate 674: tert-butyl 4-(2-(4-cyano-3-fluorophenoxy)ethyl)piperidine-1-carboxylate

To a cooled (0° C.) solution of 2-fluoro-4-hydroxybenzonitrile (CAS No. 82380-18-5, 49.4 g, 342 mmol) in acetone (1000 mL) was added tert-butyl 4-(2-bromoethyl)piperidine-1-carboxylate (Intermediate 673, 100 g, 342 mmol) followed by potassium carbonate (135.7 g, 1370 mmol) and tetrabutylammonium iodide (12.6 g, 34.2 mmol) and the reaction mixture was stirred at 50° C. for 6 h. The reaction mixture was concentrated in vacuo, diluted with water (400 mL) and extracted with ethyl acetate (3×300 mL). The combined organic layers were dried over anhydrous Na₂SO₄, filtered, and concentrated in vacuo. The crude material was purified by combi-flash chromatography, by eluting with 20% ethyl acetate in heptane, to afford tert-butyl 4-(2-(4-cyano-3-fluorophenoxy)ethyl)piperidine-1-carboxylate (Intermediate 674, 106 g, 89%) as colorless liquid. ¹H NMR (400 MHz, DMSO-d₆) δ ppm 1.02-1.10 (m, 2H), 1.38 (s, 9H), 1.61-1.68 (m, 5H), 2.64-2.67 (m, 2H), 3.90-3.92 (m, 2H), 4.11-4.17 (m, 2H), 6.97 (d, J=8.61 Hz, 1H), 7.14-7.19 (m, 1H), 7.81 (t, J=8.22 Hz, 1H).

### Step 3

### Intermediate 675: tert-butyl 4-(2-(4-carbamoyl-3-fluorophenoxy)ethyl)piperidine-1-carboxylate

To a cooled (0° C.) solution of tert-butyl 4-(2-(4-cyano-3-fluorophenoxy)ethyl)piperidine-1-carboxylate (Intermediate 674, 53.0 g, 152 mmol) in dimethyl sulfoxide (550 mL) was added potassium carbonate (42.0 g, 304 mmol) followed by 30% hydrogen peroxide (47.0 mL, 456 mmol) and the reaction mixture was stirred at room temperature for 16 h. The reaction was quenched by the addition of saturated aqueous sodium thiosulfate solution (200 mL) and diluted with ice cold water (500 mL). The resultant precipitate was collected by filtration and dried in vacuo to afford tert-butyl 4-(2-(4-carbamoyl-3-fluorophenoxy)ethyl)piperidine-1-carboxylate (Intermediate 675, 42 g, 75%) as an off-white solid, which was used for the next step without further purification. Mass spec m/z 367.1 [M+H]⁺.

### Step 4

### Intermediate 676: 2-fluoro-4-(2-(piperidin-4-yl)ethoxy)benzamide hydrochloride

To a cooled (0° C.) solution of tert-butyl 4-(2-(4-carbamoyl-3-fluorophenoxy)ethyl)piperidine-1-carboxylate (Intermediate 675, 80.0 g, 218 mmol) in 1,4-dioxane (500 mL) was added 4M hydrochloric acid in 1,4-dioxane (500 mL) and the reaction mixture was stirred at room temperature for 12 h. The reaction mixture was concentrated in vacuo, the residue triturated with diethyl ether (100 mL) and dried in vacuo to afford 2-fluoro-4-(2-(piperidin-4-yl)ethoxy)benzamide hydrochloride (Intermediate 676, 52.0 g, 89%) as an off-white solid, which was used for the next step without further purification. ¹H NMR (400 MHz, DMSO-d₆) δ ppm 1.30-1.38 (m, 2H), 1.65-1.71 (m, 3H), 1.81-1.84 (m, 2H), 2.77-2.86 (m, 2H), 3.19-3.23 (m, 2H), 4.04-4.07 (m, 2H), 6.80-6.86 (m, 2H), 7.40 (br s, 1H), 7.46 (br s, 1H), 7.65 (t, J=8.80 Hz, 1H), 8.51 (br s, 1H), 8.76 (br s, 1H). Mass spec m/z 267.0 [M+H]⁺.

### Step 5

### Intermediate 677: 4-(2-(1-(3-bromo-2-formylphenyl)piperidin-4-yl)ethoxy)-2-fluorobenzamide

To a solution of 2-fluoro-4-(2-(piperidin-4-yl)ethoxy)benzamide hydrochloride (Intermediate 676, 25 g, 93.9 mmol) in dimethyl sulfoxide (250 mL) was added N,N-diisopropylethylamine (82.0 mL, 469 mmol) followed by 2-bromo-6-fluorobenzaldehyde (CAS No. 360575-28-6, 19.1 g, 93.9 mmol) and the reaction mixture was heated to 80° C. for 12 h. The reaction mixture was diluted with water (400 mL) and extracted with ethyl acetate (3×500 mL). The combined organic layers were dried over anhydrous Na₂SO₄, filtered and concentrated in vacuo. The crude material was purified by combi-flash chromatography, by eluting with 80% ethyl acetate in heptane, to afford 4-(2-(1-(3-bromo-2-formylphenyl)piperidin-4-yl)ethoxy)-2-fluorobenzamide (Intermediate 677, 19.0 g, 45%) as a yellow semi-liquid. ¹H NMR (400 MHz, DMSO-d₆) δ ppm 1.35-1.49 (m, 2H), 1.61-1.67 (m, 2H), 1.69-1.84 (m, 4H), 3.17-3.23 (m, 2H), 4.10-4.17 (m, 2H), 6.81-6.89 (m, 1H), 6.91 (br s, 1H), 7.22 (d, J=7.83 Hz, 2H), 7.31 (d, J=7.82 Hz, 1H), 7.37-7.45 (m, 2H), 7.48 (br s, 1H), 7.67 (t, J=8.07 Hz, 1H), 10.04 (br s, 1H). Mass spec m/z 451.1 [M+H]⁺.

### Step 6

### Intermediate 678: 4-(2-(1-(3-bromo-2-(((2,6-dioxopiperidin-3-yl)amino)methyl)phenyl)piperidin-4-yl)ethoxy)-2-fluorobenzamide

To a solution of 4-(2-(1-(3-bromo-2-formylphenyl)piperidin-4-yl)ethoxy)-2-fluorobenzamide (Intermediate 677, 16.0 g, 35.61 mmol) in dimethylformamide (160 mL) was added and 3-aminopiperidine-2,6-dione hydrochloride (CAS No. 24666-56-6, 5.86 g, 35.61 mmol) and the reaction mixture was heated at 75° C. for 2 h. Sodium cyanoborohydride (7.07 g, 106.8 mmol) was then added and the reaction was heated to 80° C. for 5 h. The reaction mixture was diluted with water (100 mL) and the resultant precipitate was collected by filtration and dried in vacuo to afford 4-(2-(1-(3-bromo-2-(((2,6-dioxopiperidin-3-yl)amino)methyl)phenyl)piperidin-4-yl)ethoxy)-2-fluorobenzamide (Intermediate 678, 14.5 g, 72%) as a semi-solid which was directly taken into next step. Mass spec m/z 561.1 [M+H]⁺.

### Step 7

### Intermediate 679: 4-(2-(1-(2-(2,6-dioxopiperidin-3-yl)-1-oxoisoindolin-4-yl)piperidin-4-yl)ethoxy)-2-fluorobenzamide

To a solution of 4-(2-(1-(3-bromo-2-(((2,6-dioxopiperidin-3-yl)amino)methyl)phenyl)piperidin-4-yl)ethoxy)-2-fluorobenzamide (Intermediate 678, 8.0 g, 14.25 mmol) in 1,4-dioxane (80 mL) was added triethylamine (5.99 mL, 42.8 mmol). The reaction mixture was purged with argon for 15 min then Xantphos (2.55 g, 4.27 mmol) followed by PdCl₂(dppf) (2.19 g, 2.85 mmol) and W(CO)₆ (2.50 g, 7.12 mmol) were added. The reaction mixture was further purged with argon for 10 min and heated at 100° C. for 4 h. The reaction mixture was diluted with water (150 mL) and extracted with ethyl acetate (3×100 mL). The combined organic layers were dried over anhydrous Na₂SO₄, filtered, and concentrated in vacuo. The crude material was purified by combi-flash chromatography, by eluting with 80% ethyl acetate in heptane, to afford 4-(2-(1-(2-(2,6-dioxopiperidin-3-yl)-1-oxoisoindolin-4-yl)piperidin-4-yl)ethoxy)-2-fluorobenzamide (Intermediate 679, 6.0 g, 82%) as a yellow liquid. ¹H NMR (400 MHz, DMSO-d₆) δ ppm 1.15-1.20 (m, 3H), 1.31-1.40 (m, 2H), 1.58-1.64 (m, 2H), 1.70-1.78 (m, 1H), 1.78-1.88 (m, 1H), 1.94-2.04 (m, 1H), 2.79-2.91 (m, 1H), 3.05-3.15 (m, 3H), 3.35-3.43 (m, 1H), 3.54-3.59 (s, 1H), 4.09-4.13 (m, 1H), 4.27-4.31 (m, 1H), 4.41-4.45 (m, 1H), 5.08-5.16 (m, 1H), 6.83-6.90 (m, 1H), 7.13-7.24 (m, 2H), 7.28-7.46 (m, 3H), 7.64-7.69 (m, 1H), 8.93 (s, 1H), 10.97 (br s, 1H). Mass spec m/z 509.2 [M+H]⁺.

### Step 8

### Intermediate 680: tert-butyl 6-(4-(2-(1-(2-(2,6-dioxopiperidin-3-yl)-1-oxoisoindolin-4-yl)piperidin-4-yl)ethoxy)-2-fluorobenzamido)-2-((R)-1-methylpyrrolidin-2-yl)-1H-pyrrolo[3,2-c]pyridine-1-carboxylate

To a solution of 4-(2-(1-(2-(2,6-dioxopiperidin-3-yl)-1-oxoisoindolin-4-yl)piperidin-4-yl)ethoxy)-2-fluorobenzamide (Intermediate 679, 6.50 g, 13.0 mmol) in 1,4-dioxane (70 mL) was added tert-butyl (R)-6-bromo-2-(1-methylpyrrolidin-2-yl)-1H-pyrrolo[3,2-c]pyridine-1-carboxylate (Intermediate 4, 5.80 g, 15.0 mmol) followed by cesium carbonate (12.0 g, 38.0 mmol). The reaction mixture was purged with argon for 15 min then Pd₂(dba)₃ (1.80 g, 1.9.0 mmol) and Xantphos (2.30 g, 3.80 mmol) were added. The reaction mixture was further purged with argon for 10 min and heated at 110° C. for 8 h. The reaction mixture was filtered through a celite bed and the filter pad was washed with ethyl acetate (300 mL) and the filtrate was concentrated in vacuo. The crude material was purified by combi-flash chromatography, by eluting with 100% ethyl acetate, to afford tert-butyl 6-(4-(2-(1-(2-(2,6-dioxopiperidin-3-yl)-1-oxoisoindolin-4-yl)piperidin-4-yl)ethoxy)-2-fluorobenzamido)-2-((R)-1-methylpyrrolidin-2-yl)-1H-pyrrolo[3,2-c]pyridine-1-carboxylate (Intermediate 680, 5.0 g, 48%) as a yellow liquid. Mass spec m/z 808.3 [M+H]⁺.

### Step 9

### Example 171: 4-(2-(1-(2-(2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide

To a solution of tert-butyl 6-(4-(2-(1-(2-(2,6-dioxopiperidin-3-yl)-1-oxoisoindolin-4-yl)piperidin-4-yl)ethoxy)-2-fluorobenzamido)-2-((R)-1-methylpyrrolidin-2-yl)-1H-pyrrolo[3,2-c]pyridine-1-carboxylate (Intermediate 680, 7.0 g, 8.66 mmol) in 1,4-dioxane (70 mL) was added 4M hydrochloric acid in 1,4-dioxane (70 mL) and the reaction mixture was stirred at room temperature for 4 h then concentrated in vacuo. The crude material was purified by preparative HPLC (Method A) to afford 4-(2-(1-(2-(2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide (Example 171, 0.750 g, 12%) as an off-white solid. ¹H NMR (400 MHz, DMSO-d₆) δ ppm 1.35-1.43 (m, 2H), 1.66-1.92 (m, 9H), 1.98-2.03 (m, 1H), 2.09-2.14 (m, 1H), 2.16 (s, 3H), 2.20-2.29 (m, 1H), 2.42-2.48 (m, 1H), 2.55-2.64 (m, 1H), 2.70-2.78 (m, 2H), 2.86-2.98 (m, 1H), 3.10-3.18 (m, 1H), 3.34-3.45 (m, 2H), 4.14-4.17 (m, 2H), 4.24-4.36 (m, 1H), 4.39-4.46 (m, 1H), 5.09-5.13 (m, 1H), 6.38 (s, 1H), 6.90-6.99 (m, 2H), 7.17 (d, J=7.88 Hz, 1H), 7.30 (d, J=7.25 Hz, 1H), 7.40-7.47 (m, 1H), 7.76 (t, J=8.82 Hz, 1H), 8.19 (s, 1H), 8.46 (s, 1H), 9.95 (br s, 1H), 10.96 (br s, 1H), 11.37 (br s, 1H). Mass spec m/z 708.2 [M+H]⁺.

### Examples 172 and 173 were Prepared Following Scheme 167

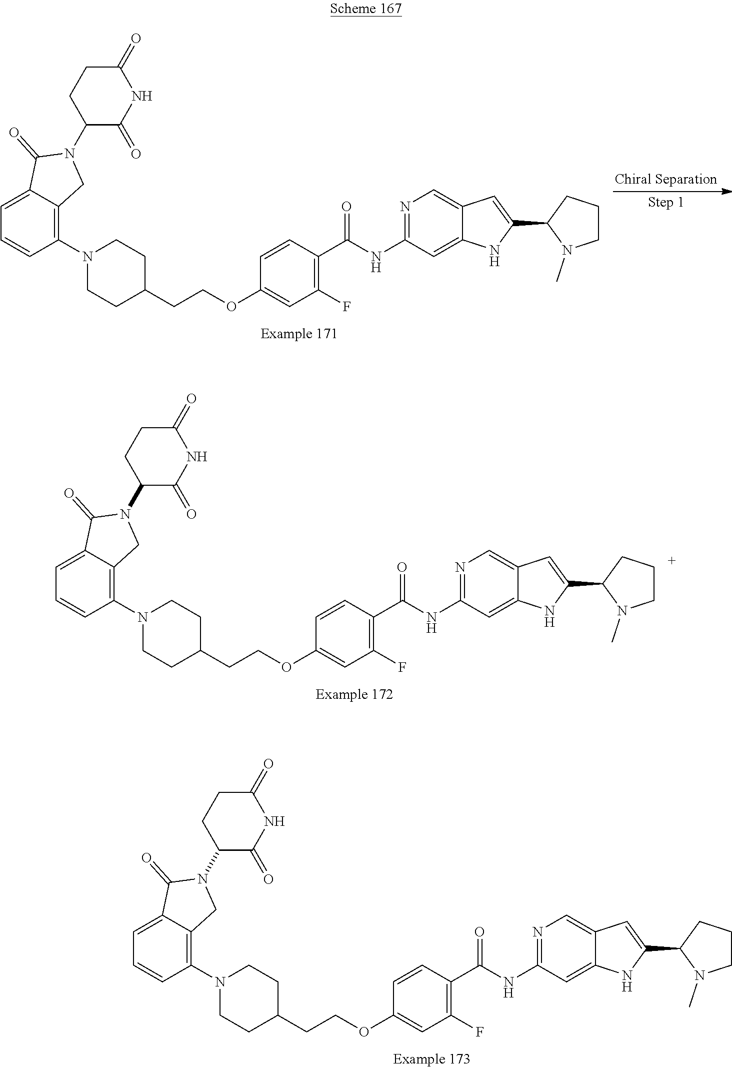

### Step 1

### Example 172: 4-(2-(1-(2-((rel-S)-2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide and Example 173: 4-(2-(1-(2-((rel-R)-2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide

4-(2-(1-(2-(2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide (Example 171, 0.15 g, 0.21 mmol) was separated by chiral preparative HPLC (Column: IC (20×250*mm), 5 m; Mobile Phase A: DCM, Mobile Phase B: iPrOH (1:1); Flow rate: 24 ml/min; isocratic 50% B) to afford 4-(2-(1-(2-((rel-S)-2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide (Example 172, 1st eluting peak, 0.021 g, 1%) as an off-white solid and 4-(2-(1-(2-((rel-R)-2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide (Example 173, 2nd eluting peak, 0.027 g, 1%) as an off-white solid.

### Example 172: 4-(2-(1-(2-((rel-S)-2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide

1^{st} eluting peak from chiral preparative HPLC. Retention time: 16.11 mins.

¹H NMR (400 MHz, DMSO-d₆) δ ppm 1.30-1.46 (m, 2H), 1.61-1.95 (m, 8H), 1.95-2.04 (m, 1H), 2.11-2.15 (m, 1H), 2.16 (s, 3H), 2.21-2.30 (m, 1H), 2.43-2.47 (m, 1H), 2.53-2.64 (m, 2H), 2.69-2.80 (m, 2H), 2.86-2.97 (m, 1H), 3.11-3.18 (m, 1H), 3.33-3.44 (m, 2H), 4.14-4.17 (m, 2H), 4.27-4.32 (m, 1H), 4.40-4.46 (m, 1H), 5.09-5.13 (m, 1H), 6.38 (s, 1H), 6.90-6.99 (m, 2H), 7.18 (d, J=7.50 Hz, 1H), 7.30 (d, J=7.00 Hz, 1H), 7.40-7.45 (m, 1H), 7.76 (t, J=8.82 Hz, 1H), 8.19 (s, 1H), 8.46 (s, 1H), 9.96 (br s, 1H), 10.98 (s, 1H), 11.38 (s, 1H). Mass spec m/z 708.3 [M+H]⁺.

### Example 173: 4-(2-(1-(2-((rel-R)-2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide

2nd eluting peak from chiral preparative HPLC. Retention time: 24.68 min.

¹H NMR (400 MHz, DMSO-d₆) δ ppm 1.33-1.43 (m, 1H), 1.64-1.72 (m, 1H), 1.74-1.78 (m, 2H), 1.81-1.94 (m, 6H), 1.95-2.04 (m, 1H), 2.12-2.15 (m, 1H), 2.16 (s, 3H), 2.21-2.30 (m, 1H), 2.43-2.47 (m, 1H), 2.53-2.64 (m, 2H), 2.69-2.80 (m, 2H), 2.86-2.97 (m, 1H), 3.11-3.18 (m, 1H), 3.33-3.44 (m, 2H), 4.14-4.17 (m, 2H), 4.27-4.32 (m, 1H), 4.42-4.48 (m, 1H), 5.09-5.13 (m, 1H), 6.38 (s, 1H), 6.90-6.99 (m, 2H), 7.18 (d, J=7.50 Hz, 1H), 7.30 (d, J=7.00 Hz, 1H), 7.40-7.45 (m, 1H), 7.76 (t, J=8.82 Hz, 1H), 8.19 (s, 1H), 8.46 (s, 1H), 9.96 (br s, 1H), 10.98 (s, 1H), 11.38 (s, 1H). Mass spec m/z 708.3 [M+H]⁺.

### Example 174 was Prepared Following Scheme 168

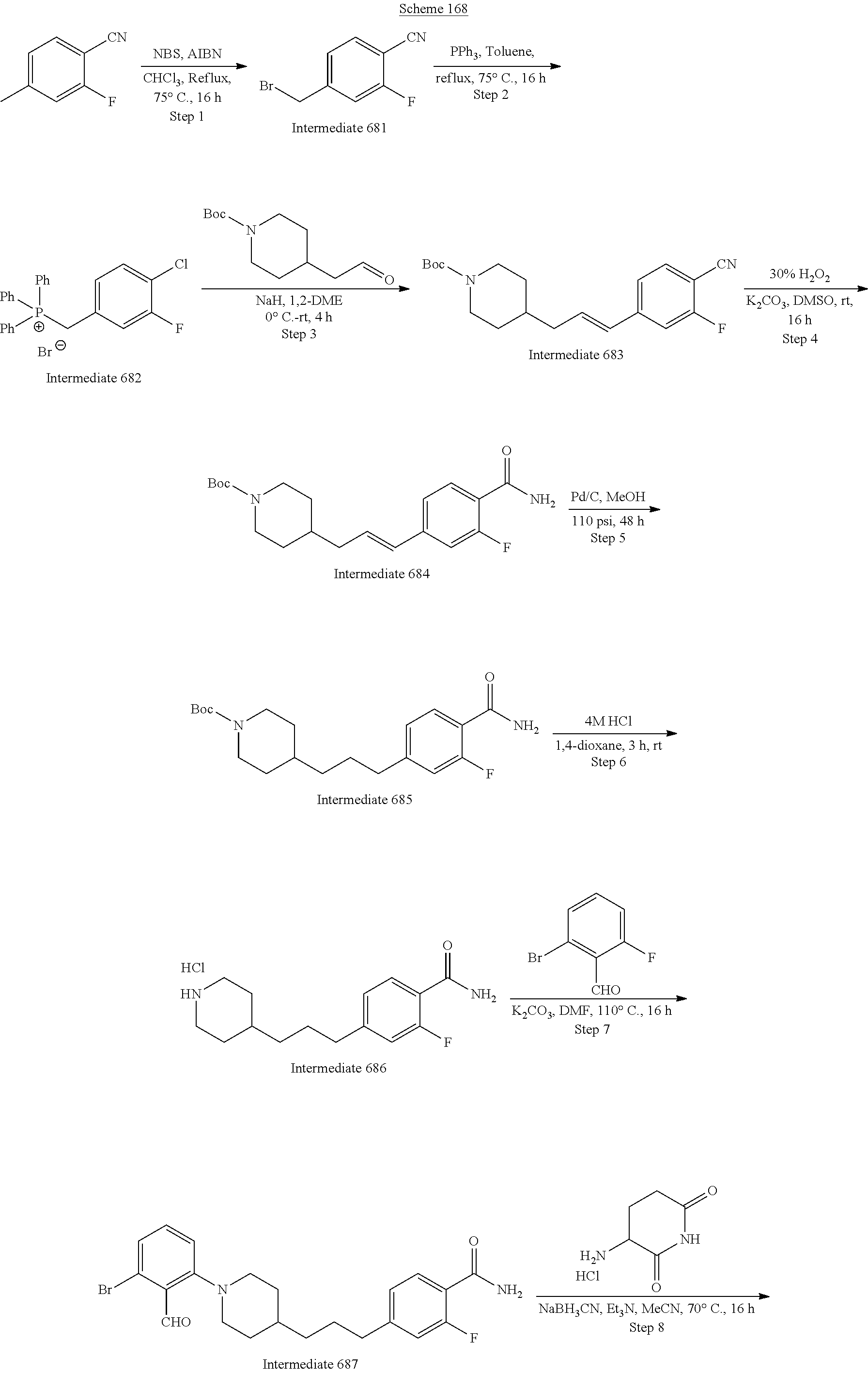

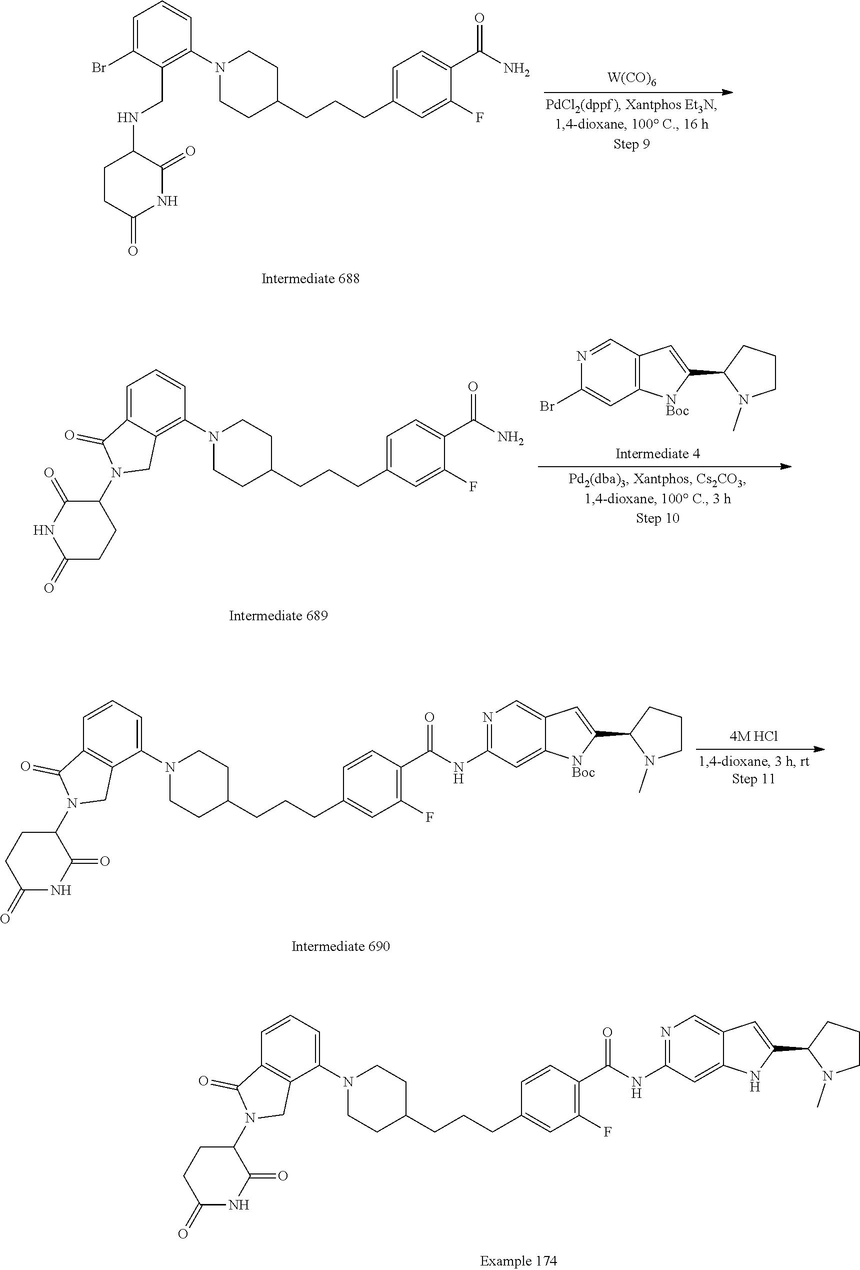 [SMILES: NC(=O)c1ccc(CCCC2CCN(c3cccc(Br)c3CNC3CCC(=O)NC3=O)CC2)cc1F ; "W(CO)6" ; NC(=O)c1ccc(CCCC2CCN(c3cccc4c3CN(C3CCC(=O)NC3=O)C4=O)CC2)cc1F ; CN1CCC[C@@H]1c1cc2cnc(Br)cc2n1C(=O)OC(C)(C)C ; CN1CCC[C@@H]1c1cc2cnc(NC(=O)c3ccc(CCCC4CCN(c5cccc6c5CN(C5CCC(=O)NC5=O)C6=O)CC4)cc3F)cc2n1C(=O)OC(C)(C)C ; CN1CCC[C@@H]1c1cc2cnc(NC(=O)c3ccc(CCCC4CCN(c5cccc6c5CN(C5CCC(=O)NC5=O)C6=O)CC4)cc3F)cc2[nH]1]

### Step 1

### Intermediate 681: 4-(bromomethyl)-2-fluorobenzonitrile

To a solution of 2-fluoro-4-methylbenzonitrile (CAS No. 85070-67-3, 25.0 g, 179.4 mmol) in 1,2 dichloroethane (250 mL) was added 2,2′-azobis(2-methylpropionitrile) (3.03 g, 17.9 mmol) followed by N-bromosuccinimide (35.85 g, 197.4 mmol) and the reaction mixture was heated at 75° C. for 16 h. The reaction mixture was then quenched with water (500 mL) and extracted with dichloromethane (2×500 mL). The combined organic layers were dried over anhydrous Na₂SO₄, filtered and concentrated in vacuo. The crude material was purified by combi-flash chromatography, by eluting with 5% ethyl acetate in heptane, to afford 4-(bromomethyl)-2-fluorobenzonitrile (Intermediate 681, 27.0 g, 70%) as pale-yellow oil. ¹H NMR (400 MHz, DMSO-d₆) δ ppm 4.75 (m, 2H), 7.50 (d, J=8.0 Hz, 1H), 7.63 (d, J=10.4 Hz, 1H), 7.93 (t, J=7.60 Hz, 1H).

### Step 2

### Intermediate 682: (4-cyano-3-fluorobenzyl)triphenylphosphonium bromide

To a solution of 4-(bromomethyl)-2-fluorobenzonitrile (Intermediate 681, 30.0 g, 137.4 mmol) in toluene (300 mL) was added triphenylphosphine (44.1 g, 164.8 mmol) and the reaction mixture was heated at 120° C. for 16 h. The reaction mixture was filtered and the solid precipitate was washed with ethyl acetate (200 mL) and dried in vacuo to afford (4-cyano-3-fluorobenzyl)triphenylphosphonium bromide (Intermediate 682, 40.0 g, 73%) as an off-white solid, which was used for the next step without further purification. ¹H NMR (400 MHz, DMSO-d₆), δ ppm 5.31-5.43 (m, 2H), 6.99-7.09 (m, 2H), 7.68-7.98 (m, 16H). Mass spec m/z 396.2 [M+H]⁺.

### Step 3

### Intermediate 683: tert-butyl (E)-4-(3-(4-cyano-3-fluorophenyl)allyl)piperidine-1-carboxylate

To a cooled (0° C.) solution of (4-cyano-3-fluorobenzyl)triphenylphosphonium bromide (Intermediate 682, 20.5 g, 51.7 mmol) in 1,2-dimethoxyethane (200 mL) was added sodium hydride (60% in mineral oil, 1.89 g, 47.3 mmol) and the reaction mixture was stirred at 0° C. for 30 min. tert-Butyl 4-(2-oxoethyl)piperidine-1-carboxylate (CAS No: 142374-19-4, 10 g, 43.1 mmol) was then added and the reaction mixture was stirred at room temperature for 4 h. The reaction mixture was quenched with water (200 mL) and extracted with ethyl acetate (2×500 mL). The combined organic layers were dried over anhydrous Na₂SO₄, filtered and concentrated in vacuo. The crude material was purified by combi-flash chromatography, by eluting with 18% ethyl acetate in heptane, to afford tert-butyl (E)-4-(3-(4-cyano-3-fluorophenyl)allyl)piperidine-1-carboxylate (Intermediate 683, 5.0 g, 31%) as a pale-yellow oil. ¹H NMR (400 MHz, DMSO-d₆) δ ppm 1.04-1.28 (m, 2H), 1.48 (s, 9H), 1.50-1.59 (m, 1H), 1.65-1.70 (m, 2H), 2.19-2.24 (m, 2H), 2.60-2.74 (m, 2H), 4.00-4.11 (m, 2H), 5.83-5.89 (m, 1H), 6.43 (d, J=8.4 Hz, 1H), 7.07-7.18 (m, 2H), 7.50-7.58 (m, 1H).

### Step 4

### Intermediate 684: tert-butyl (E)-4-(3-(4-carbamoyl-3-fluorophenyl)allyl)piperidine-1-carboxylate

To a cooled (0° C.) solution of tert-butyl (E)-4-(3-(4-cyano-3-fluorophenyl)allyl)piperidine-1-carboxylate (Intermediate 683, 6.0 g, 17.4 mmol) in dimethyl sulfoxide (40 mL) was added potassium carbonate (6.01 g, 43.6 mmol) and 30% hydrogen peroxide (13.6 mL, 174.2 mmol) and the reaction mixture was stirred at room temperature for 16 h. The reaction mixture was quenched with ice cold water (300 mL), the resultant precipitate was collected by filtration and dried in vacuo to afford tert-butyl (E)-4-(3-(4-carbamoyl-3-fluorophenyl)allyl)piperidine-1-carboxylate (Intermediate 684, 5.0 g, 79%) as an off-white solid, which was used for the next step without further purification. ¹H NMR (400 MHz, DMSO-d₆), δ ppm 0.95-1.05 (m, 2H), 1.38 (s, 9H), 1.53-1.69 (m, 3H), 2.11-2.29 (m, 2H), 2.53-2.56 (m, 2H), 3.90-3.93 (m, 2H), 5.74-5.85 (m, 1H), 6.44-6.49 (m, 1H), 7.05-7.35 (m, 3H), 7.54-7.66 (m, 2H). Mass spec m/z 363.2 [M+H]⁺.

### Step 5

### Intermediate 685: tert-butyl 4-(3-(4-carbamoyl-3-fluorophenyl)propyl)piperidine-1-carboxylate

To a solution of tert-butyl (E)-4-(3-(4-carbamoyl-3-fluorophenyl)allyl)piperidine-1-carboxylate (Intermediate 684, 6.5 g, 17.9 mmol) in methanol (70 mL) was added 10% Pd/C (1.91 g, 17.9 mmol). The reaction mixture was stirred at room temperature for 48 h under under 110 psi H₂ atmosphere. The reaction mixture was filtered through a celite bed, the filter pad was washed with methanol (150 mL) and the filtrate concentrated in vacuo to afford tert-butyl 4-(3-(4-carbamoyl-3-fluorophenyl)propyl)piperidine-1-carboxylate (Intermediate 685, 5.0 g, 76%) as colourless oil, which was used for the next step without further purification. ¹H NMR (400 MHz, DMSO-d₆), δ ppm 0.90-0.93 (m, 2H), 1.20-1.22 (m, 2H), 1.38 (s, 9H), 1.59-1.61 (4H), 2.57-2.68 (m, 3H), 2.97-3.03 (m, 2H), 3.88-3.91 (m, 2H), 7.06-7.14 (m, 2H), 7.49-7.64 (m, 3H). Mass spec m/z 365.2 [M+H]⁺.

### Step 6

### Intermediate 686: 2-fluoro-4-(3-(piperidin-4-yl)propyl)benzamide hydrochloride

To a cooled (0° C.) solution of tert-butyl 4-(3-(4-carbamoyl-3-fluorophenyl)propyl)piperidine-1-carboxylate (Intermediate 685, 4.8 g, 13.0 mmol) in dichloromethane (45 mL) was added 4M hydrochloric acid in 1,4-dioxane (40 mL) and the reaction mixture was stirred at room temperature for 3 h. The reaction mixture was then concentrated in vacuo and the crude residue was triturated with diethyl ether (2×50 mL), then dried in vacuo to afford 2-fluoro-4-(3-(piperidin-4-yl)propyl)benzamide hydrochloride (Intermediate 686, 5.0 g, 77%) as an off-white solid, which was used for the next step without further purification. ¹H NMR (400 MHz, DMSO-d₆), δ ppm 1.19-1.28 (m, 4H), 1.49-1.57 (m, 4H), 1.73-1.84 (2H), 2.56-2.61 (m, 3H), 2.74-2.84 (m, 2H), 3.16-3.21 (m, 2H), 7.06-7.13 (m, 1H), 7.49-7.64 (m, 2H), 8.54 (br s, 1H), 8.78 (br s, 1H). Mass spec m/z 265.4 [M+H]⁺.

### Step 7

### Intermediate 687: 4-(3-(1-(3-bromo-2-formylphenyl)piperidin-4-yl)propyl)-2-fluorobenzamide

To a solution of 2-fluoro-4-(3-(piperidin-4-yl)propyl)benzamide hydrochloride (Intermediate 686, 5.0 g, 16.6 mmol) in dimethylformamide (30 mL) was added potassium carbonate (6.89 g, 49.9 mmol) and 2-bromo-6-fluorobenzaldehyde (CAS No. 360575-28-6, 3.51 g, 16.6 mmol) and the reaction mixture was heated at 110° C. for 16 h. The reaction mixture was diluted with ice cold water (400 mL), the resultant precipitate was collected by filtration, washed with heptane (2×100 mL), and a dried in vacuo to afford 4-(3-(1-(3-bromo-2-formylphenyl)piperidin-4-yl)propyl)-2-fluorobenzamide (Intermediate 687, 5.1 g, 58%) as a light blue solid, which was used for the next step without further purification. ¹H NMR (400 MHz, DMSO-d₆), δ ppm 1.23-1.35 (m, 5H), 1.60-1.63 (m, 2H), 1.72-1.75 (m, 2H), 2.60-2.64 (m, 2H), 2.82-2.87 (m, 2H), 3.13-3.16 (m, 2H), 7.10-7.16 (m, 2H), 7.20 (d, J=8.02 Hz, 1H), 7.30 (d, J=7.73 Hz, 1H), 7.40 (t, J=7.97 Hz, 1H), 7.54-7.62 (m, 3H), 10.01 (s, 1H). Mass spec m/z 449.1 [M+H+2]⁺.

### Step 8

### Intermediate 688: 4-(3-(1-(3-bromo-2-(((2,6-dioxopiperidin-3-yl)amino)methyl)phenyl)piperidin-4-yl)propyl)-2-fluorobenzamide

To a solution of 4-(3-(1-(3-bromo-2-formylphenyl)piperidin-4-yl)propyl)-2-fluorobenzamide (Intermediate 687, 4.0 g, 8.94 mmol) in acetonitrile (10 mL) was added 3-aminopiperidine-2,6-dione hydrochloride (CAS No. 24666-56-6, 1.62 g, 9.84 mmol) followed by triethylamine (3.76 mL, 26.8 mmol) and the reaction mixture was stirred at room temperature for 10 min. Sodium cyanoborohydride (1.77 g, 26.8 mmol) was then added and the reaction mixture was heated at 70° C. for 6 h. The reaction mixture was then concentrated in vacuo and diluted with ice cold water (400 mL). The resultant precipitate was collected by filtration, washed with diethyl ether (2×50 mL), and dried in vacuo to afford to afford 4-(3-(1-(3-bromo-2-(((2,6-dioxopiperidin-3-yl)amino)methyl)phenyl)piperidin-4-yl)propyl)-2-fluorobenzamide (Intermediate 688, 4.0 g, 79%) as a green solid. ¹H NMR (400 MHz, DMSO-d₆), δ ppm 1.20-1.48 (m, 5H), 1.60-1.77 (m, 6H), 2.24-2.25 (m, 2H), 2.60-2.74 (m, 4H), 2.90-3.07 (m, 2H), 3.17-3.30 (m, 2H), 3.84-3.91 (m, 2H), 7.08-7.18 (m, 4H), 7.27-7.31 (m, 1H), 7.53-7.59 (m, 3H), 10.76 (s, 1H). Mass spec m/z 561.2 [M+H+2]⁺.

### Step 9

### Intermediate 689: 4-(3-(1-(2-(2,6-dioxopiperidin-3-yl)-1-oxoisoindolin-4-yl)piperidin-4-yl)propyl)-2-fluorobenzamide

To a solution of 4-(3-(1-(3-bromo-2-(((2,6-dioxopiperidin-3-yl)amino)methyl)phenyl)piperidin-4-yl)propyl)-2-fluorobenzamide (Intermediate 688, 4.0 g, 7.15 mmol) in 1,4-dioxane (40 mL) was added triethylamine (3.0 mL, 21.4 mmol). The reaction mixture was purged with argon for 15 min then Xantphos (0.85 g, 1.43 mmol) followed by PdCl₂(dppf) (1.10 g, 1.43 mmol) and W(CO)₆ (1.3 g, 3.57 mmol) were added. The reaction mixture was further purged with argon for 10 min and heated at 100° C. for 16 h. The reaction mixture was concentrated in vacuo and the crude material was purified by combi-flash chromatography, by eluting with 12% MeOH in DCM, to afford 4-(3-(1-(2-(2,6-dioxopiperidin-3-yl)-1-oxoisoindolin-4-yl)piperidin-4-yl)propyl)-2-fluorobenzamide (Intermediate 689, 2.1 g, 58%) as an off-white solid. ¹H NMR (400 MHz, DMSO-d₆), δ ppm 1.24-1.27 (m, 4H), 1.32-1.47 (m, 1H), 1.62-1.65 (m, 3H), 1.73-1.79 (m, 3H), 1.95-1.99 (m, 1H), 2.51-2.72 (m, 6H), 2.82-2.94 (m, 1H), 4.25-4.29 (m, 1H), 4.39-4.43 (m, 1H), 5.09-5.12 (m, 1H), 7.10-7.16 (m, 3H), 7.26-7.30 (m, 1H), 7.38-7.43 (m, 1H), 7.57-7.61 (m, 3H), 10.98 (s, 1H). Mass spec m/z 507.2 [M+H]⁺.

### Step 10

### Intermediate 690: tert-butyl 6-(4-(3-(1-(2-(2,6-dioxopiperidin-3-yl)-1-oxoisoindolin-4-yl)piperidin-4-yl)propyl)-2-fluorobenzamido)-2-((R)-1-methylpyrrolidin-2-yl)-1H-pyrrolo[3,2-c]pyridine-1-carboxylate

To a solution of 4-(3-(1-(2-(2,6-dioxopiperidin-3-yl)-1-oxoisoindolin-4-yl)piperidin-4-yl)propyl)-2-fluorobenzamide (Intermediate 689, 0.30 g, 0.59 mmol) in 1,4-dioxane (2 mL) was added tert-butyl (R)-6-bromo-2-(1-methylpyrrolidin-2-yl)-1H-pyrrolo[3,2-c]pyridine-1-carboxylate (Intermediate 4, 0.24 g, 0.65 mmol) and cesium carbonate (0.57 g, 1.77 mmol). The reaction mixture was purged with argon for 15 min then Pd₂(dba)₃ (0.083 g, 0.08 mmol) and Xantphos (0.10 g, 1.77 mmol) were added. The reaction mixture was further purged with argon for 10 min and heated at 100° C. for 3 h. The reaction mixture was concentrated in vacuo and the crude material was purified by combi-flash chromatography, by eluting with 100% ethyl acetate, to afford tert-butyl 6-(4-(3-(1-(2-(2,6-dioxopiperidin-3-yl)-1-oxoisoindolin-4-yl)piperidin-4-yl)propyl)-2-fluorobenzamido)-2-((R)-1-methylpyrrolidin-2-yl)-1H-pyrrolo[3,2-c]pyridine-1-carboxylate (Intermediate 690, 0.28 g, 28%) as a pale yellow solid. Mass spec m/z 806.8 [M+H]⁺.

### Step 11

### Example 174: 4-(3-(1-(2-(2,6-dioxopiperidin-3-yl)-1-oxoisoindolin-4-yl)piperidin-4-yl)propyl)-2-fluoro-N-(2-((R)-1-methylpyrrolidin-2-yl)-1H-pyrrolo[3,2-c]pyridin-6-yl)benzamide

To a cooled (0° C.) solution of tert-butyl 6-(4-(3-(1-(2-(2,6-dioxopiperidin-3-yl)-1-oxoisoindolin-4-yl)piperidin-4-yl)propyl)-2-fluorobenzamido)-2-((R)-1-methylpyrrolidin-2-yl)-1H-pyrrolo[3,2-c]pyridine-1-carboxylate (Intermediate 690, 0.42 g, 0.52 mmol) in dichloromethane (4 mL) was added 4M hydrochloric acid in 1,4-dioxane (4 mL) and the reaction mixture was stirred at room temperature for 3 h. The reaction mixture was then concentrated in vacuo and the residue was triturated with diethyl ether (2×20 mL). The resultant crude solid was purified by preparative HPLC (Method A) to afford 4-(3-(1-(2-(2,6-dioxopiperidin-3-yl)-1-oxoisoindolin-4-yl)piperidin-4-yl)propyl)-2-fluoro-N-(2-((R)-1-methylpyrrolidin-2-yl)-1H-pyrrolo[3,2-c]pyridin-6-yl)benzamide (Example 174, 0.17 g, 3%) as an off-white solid. ¹H NMR (400 MHz, DMSO-d₆), δ ppm 1.22-1.43 (m, 6H), 1.61-1.71 (m, 2H), 1.65-2.01 (m, 6H), 2.12-2.19 (m, 4H), 2.21-2.29 (m, 1H), 2.43-2.78 (m, 1H), 2.53-2.59 (m, 1H), 2.67-2.84 (m, 4H), 2.86-3.00 (m, 1H) 3.11-3.18 (m, 1H), 3.28-3.30 (m, 1H), 3.32-3.41 (m, 1H), 4.25-4.30 (m, 1H), 4.39-4.44 (m, 1H), 5.09-5.13 (m, 1H), 6.39 (s, 1H), 7.14-7.21 (m, 3H), 7.28 (d, J=7.2 Hz, 1H), 7.42 (t, J=7.20 Hz, 1H), 7.68 (t, J=7.20 Hz, 1H), 8.19 (s, 1H), 8.46 (s, 1H), 10.21 (d, J=4.0 Hz, 1H), 10.98 (s, 1H), 11.39 (s, 1H). Mass spec m/z 706.3 [M+H]⁺.

### Examples 175 and 176 were Prepared Following Scheme 169

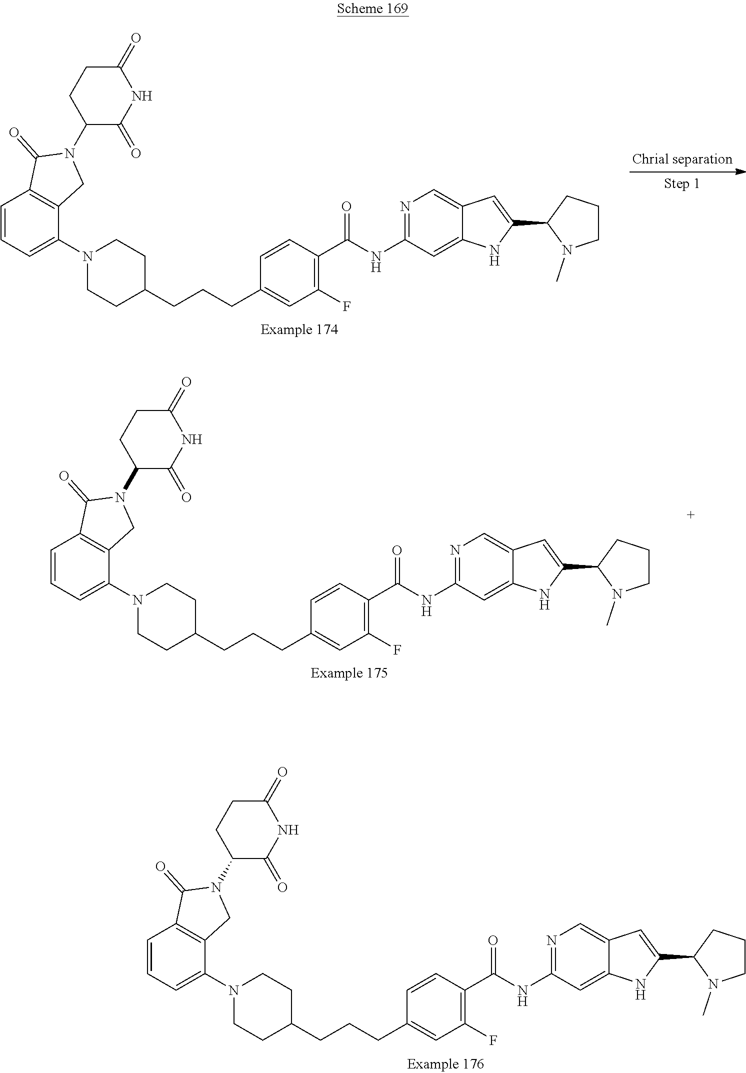 [SMILES: CN1CCC[C@@H]1c1cc2cnc(NC(=O)c3ccc(CCCC4CCN(c5cccc6c5CN(C5CCC(=O)NC5=O)C6=O)CC4)cc3F)cc2[nH]1 ; CN1CCC[C@@H]1c1cc2cnc(NC(=O)c3ccc(CCCC4CCN(c5cccc6c5CN([C@H]5CCC(=O)NC5=O)C6=O)CC4)cc3F)cc2[nH]1 ; CN1CCC[C@@H]1c1cc2cnc(NC(=O)c3ccc(CCCC4CCN(c5cccc6c5CN([C@@H]5CCC(=O)NC5=O)C6=O)CC4)cc3F)cc2[nH]1]

### Step 1

### Example 175: 4-(3-(1-(2-((rel-S)-2,6-dioxopiperidin-3-yl)-1-oxoisoindolin-4-yl)piperidin-4-yl)propyl)-2-fluoro-N-(2-((R)-1-methylpyrrolidin-2-yl)-1H-pyrrolo[3,2-c]pyridin-6-yl)benzamide and Example 176: 4-(3-(1-(2-((rel-R)-2,6-dioxopiperidin-3-yl)-1-oxoisoindolin-4-yl)piperidin-4-yl)propyl)-2-fluoro-N-(2-((R)-1-methylpyrrolidin-2-yl)-1H-pyrrolo[3,2-c]pyridin-6-yl)benzamide

4-(3-(1-(2-(2,6-dioxopiperidin-3-yl)-1-oxoisoindolin-4-yl)piperidin-4-yl)propyl)-2-fluoro-N-(2-((R)-1-methylpyrrolidin-2-yl)-1H-pyrrolo[3,2-c]pyridin-6-yl)benzamide (Example 174, 0.09 g, 0.12 mmol) was separated by chiral preparative HPLC (Column: Chiral Pak IK (10×250*mm), 5 M; Mobile Phase A: DCM, Mobile Phase B: iPrOH (1:1); Flow rate: 4 ml/min; isocratic 50% B) to afford 4-(3-(1-(2-((rel-S)-2,6-dioxopiperidin-3-yl)-1-oxoisoindolin-4-yl)piperidin-4-yl)propyl)-2-fluoro-N-(2-((R)-1-methylpyrrolidin-2-yl)-1H-pyrrolo[3,2-c]pyridin-6-yl)benzamide (Example 175, 1st eluting peak, 0.014 g, 16%) as an off-white solid and 4-(3-(1-(2-((rel-R)-2,6-dioxopiperidin-3-yl)-1-oxoisoindolin-4-yl)piperidin-4-yl)propyl)-2-fluoro-N-(2-((R)-1-methylpyrrolidin-2-yl)-1H-pyrrolo[3,2-c]pyridin-6-yl)benzamide (Example 176, 2nd eluting peak, 0.019 g, 21%) as an off-white solid.

### Example 175: 4-(3-(1-(2-((rel-S)-2,6-dioxopiperidin-3-yl)-1-oxoisoindolin-4-yl)piperidin-4-yl)propyl)-2-fluoro-N-(2-((R)-1-methylpyrrolidin-2-yl)-1H-pyrrolo[3,2-c]pyridin-6-yl)benzamide

1^{st} eluting peak from chiral preparative HPLC. Retention time: 7.24 mins.

¹H NMR (400 MHz, DMSO-d₆) δ ppm 1.23-1.36 (m, 5H), 1.37-1.52 (m, 1H), 1.60-1.72 (m, 2H), 1.73-1.82 (m, 3H), 1.82-1.94 (m, 2H), 1.90-2.04 (m, 1H), 2.11-2.15 (m, 1H), 2.16 (s, 3H), 2.21-2.30 (m, 1H), 2.55-2.63 (m, 2H), 2.68-2.77 (m, 2H), 2.86-2.97 (m, 1H), 3.11-3.15 (m, 1H), 3.21-3.29 (m, 2H), 3.37-3.42 (m, 2H), 4.24-4.32 (m, 1H), 4.38-4.46 (m, 1H), 5.08-5.13 (m, 1H), 6.39 (s, 1H), 7.13-7.23 (m, 3H), 7.29 (d, J=7.13 Hz, 1H), 7.39-7.45 (m, 1H), 7.68 (t, J=7.82 Hz, 1H), 8.19 (s, 1H), 8.46 (s, 1H), 10.19 (br s, 1H), 10.97 (br s, 1H), 11.38 (br s, 1H). Mass spec m/z 706.4 [M+H]⁺.

### Example 176: 4-(3-(1-(2-((rel-R)-2,6-dioxopiperidin-3-yl)-1-oxoisoindolin-4-yl)piperidin-4-yl)propyl)-2-fluoro-N-(2-((R)-1-methylpyrrolidin-2-yl)-1H-pyrrolo[3,2-c]pyridin-6-yl)benzamide

2nd eluting peak from chiral preparative HPLC. Retention time: 9.37 mins.

¹H NMR (400 MHz, DMSO-d₆) δ ppm 1.23-1.36 (m, 6H), 1.37-1.52 (m, 1H), 1.60-1.72 (m, 2H), 1.73-1.82 (m, 3H), 1.82-1.94 (m, 2H), 1.90-2.04 (m, 1H), 2.11-2.15 (m, 1H), 2.16 (s, 3H), 2.21-2.30 (m, 1H), 2.55-2.63 (m, 2H), 2.68-2.77 (m, 2H), 2.86-2.97 (m, 1H), 3.11-3.15 (m, 1H), 3.37-3.42 (m, 2H), 4.24-4.32 (m, 1H), 4.38-4.46 (m, 1H), 5.08-5.13 (m, 1H), 6.39 (s, 1H), 7.14-7.21 (m, 3H), 7.27 (d, J=7.13 Hz, 1H), 7.40-7.45 (m, 1H), 7.68 (t, J=7.82 Hz, 1H), 8.19 (s, 1H), 8.41 (s, 1H), 8.46 (s, 1H), 10.19 (br s, 1H), 10.97 (br s, 1H), 11.38 (br s, 1H). Mass spec m/z 706.3 [M+H]⁺.

### Example 177 was Synthesised Following Scheme 170

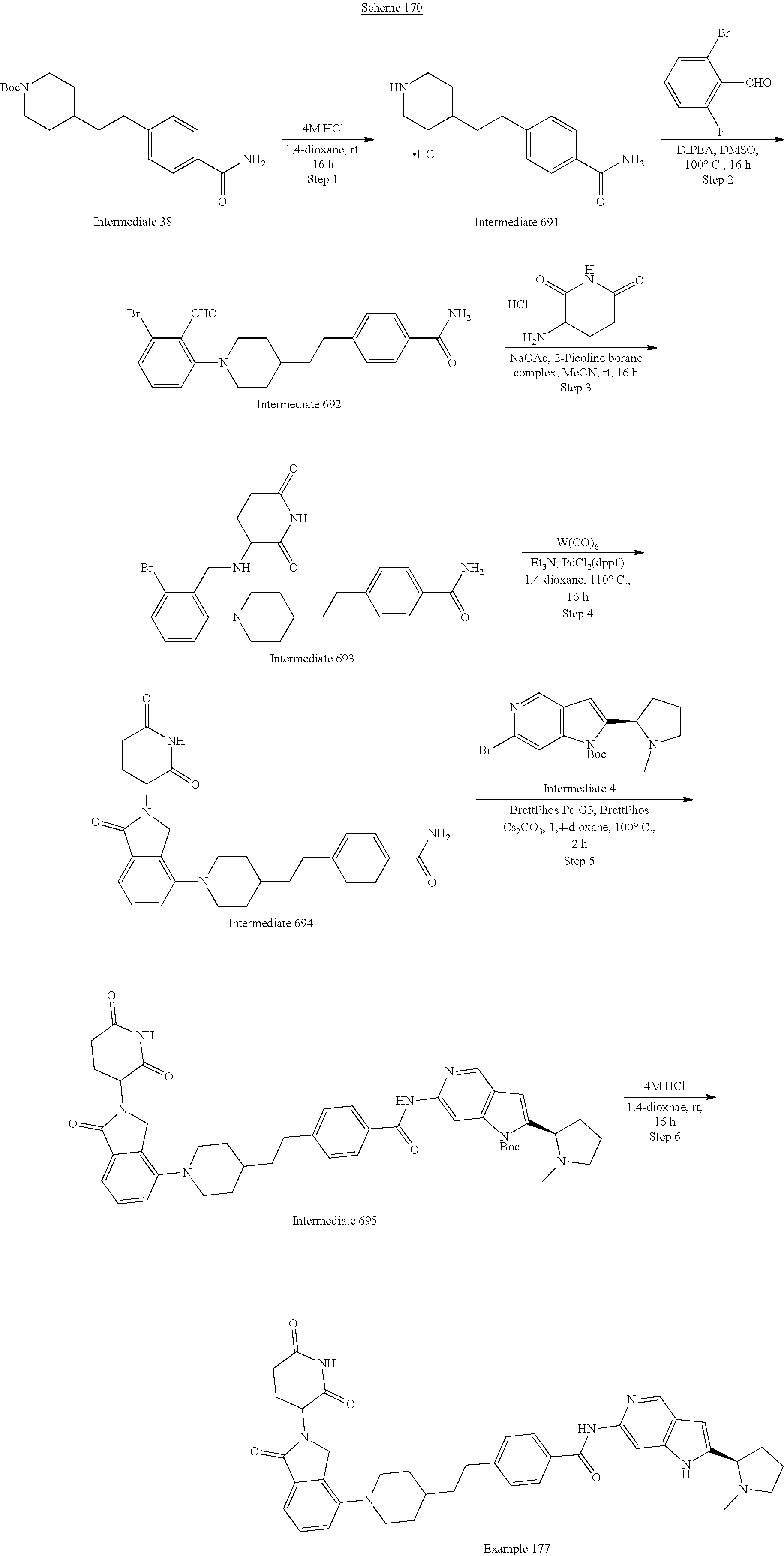

### Step 1

### Intermediate 691: 4-(2-(piperidin-4-yl)ethyl)benzamide hydrochloride

To a cooled (0° C.) solution of tert-butyl 4-(4-carbamoylphenethyl)piperidine-1-carboxylate (Intermediate 38, 2.0 g, 6.02 mmol) in 1,4-dioxane (10 mL) was added 4M hydrochloric acid in 1,4-dioxane (30 mL) and the reaction mixture was stirred at room temperature for 16 h. The reaction mixture was then concentrated in vacuo and to afford 4-(2-(piperidin-4-yl)ethyl)benzamide hydrochloride (Intermediate 691, 2.0 g,) as an off-white solid, which was used for next step without further purification. ¹H NMR (400 MHz, DMSO-d₆) δ ppm 1.33-1.40 (m, 2H), 1.43-1.56 (m, 3H), 1.81-1.85 (m, 2H), 2.62-2.68 (m, 2H), 2.63-2.78 (m, 2H), 3.18-3.23 (m, 3H), 7.28 (d, J=7.6 Hz, 2H), 7.79 (d, J=7.6 Hz, 2H), 8.22 (bs, 1H), 8,96 (bs, 1H). Mass spec m/z 231.3 [M−H]⁻.

### Step 2

### Intermediate 692: 4-(2-(1-(3-bromo-2-formylphenyl)piperidin-4-yl)ethyl)benzamide

To a solution of 4-(2-(piperidin-4-yl)ethyl)benzamide hydrochloride (Intermediate 691, 2.0 g, 7.44 mmol) in dimethyl sulfoxide (100 mL) were added 2-bromo-6-fluorobenzaldehyde (CAS No: 360575-28-6, 1.51 g, 7.44 mmol) and N,N-diisopropylethylamine (6.50 mL, 37.2 mmol) and the resulting mixture was heated at 100° C. for 16 h. After completion, the reaction mixture was quenched with ice cold water (200 mL), resulting in a solid precipitate, which was collected by filtration and dried in vacuo to afford 4-(2-(1-(3-bromo-2-formylphenyl)piperidin-4-yl)ethyl)benzamide (Intermediate 692, 3.0 g, 50%) as an off-white solid. ¹H NMR (400 MHz, DMSO-d₆) δ ppm 0.90-0.92 (m, 2H), 1.30-1.39 (m, 2H), 1.53-1.59 (m, 1H), 1.76-1.79 (m, 1H), 2.51-2.58 (m, 2H), 2.61-2.68 (m, 2H), 2.74-2.83 (m, 1H), 3.12-3.15 (m, 2H), 7.15-7.37 (m, 6H), 7.75-7.85 (m, 3H), 10.00 (s, 1H). Mass spec m/z 417.8 [M+H+2]⁺.

### Step 3

### Intermediate 693: 4-(2-(1-(3-bromo-2-(((2,6-dioxopiperidin-3-yl)amino)methyl)phenyl)piperidin-4-yl)ethyl)benzamide

To a solution of 4-(2-(1-(3-bromo-2-formylphenyl)piperidin-4-yl)ethyl)benzamide (Intermediate 692, 2.0 g, 4.8 mmol) in acetonitrile (20 mL) were added 3-aminopiperidine-2,6-dione hydrochloride (CAS No: 24666-56-6, 0.79 g, 4.8 mmol), sodium acetate (0.60 g, 7.2 mmol) followed by borane-2-picoline complex (CAS No: 3999-38-0, 1.1 g, 9.6 mmol) and the reaction was stirred at room temperature for 16 h. After completion, the reaction mixture was concentrated in vacuo to obtain the crude product. The crude material was purified by combi-flash chromatography, by eluting with ethyl acetate, to afford 4-(2-(1-(3-bromo-2-(((2,6-dioxopiperidin-3-yl)amino)methyl)phenyl)piperidin-4-yl)ethyl)benzamide (Intermediate 693, 10 g, 28%) as a white solid. ¹H NMR (400 MHz, DMSO-d₆) δ ppm 1.30-1.36 (m, 3H), 1.56-1.61 (m, 2H), 1.66-1.89 (m, 4H), 2.28-2.31 (m, 1H), 2.47-2.49 (m, 1H), 2.51-2.56 (m, 2H), 2.67-2.68 (m, 3H), 3.06-3.08 (m, 1H), 3.21-2-3.24 (m, 2H), 3.86-3.96 (m, 2H), 7.13-7.32 (m, 6H), 7.77-7.86 (m, 3H), 10.76 (s, 1H). Mass spec m/z 529.1 [M+H+2]⁺.

### Step 4

### Intermediate 694: 4-(2-(1-(2-(2,6-dioxopiperidin-3-yl)-1-oxoisoindolin-4-yl)piperidin-4-yl)ethyl)benzamide

To a solution of 4-(2-(1-(3-bromo-2-(((2,6-dioxopiperidin-3-yl)amino)methyl)phenyl)piperidin-4-yl)ethyl)benzamide (Intermediate 693, 1.0 g, 1.89 mmol) in 1,4-dioxane (10 mL) was added triethylamine (0.80 mL, 5.68 mmol) and the reaction mixture was purged with argon for 15 min. Xanthphos (0.34 g, 0.57 mmol), Pd(dppf)Cl₂ (0.15 g, 0.19 mmol) followed by tungsten hexacarbonyl (0.069 g, 0.19 mmol) were then added and the reaction mixture was purged with argon for another 10 min and then stirred at 110° C. for 16 h. After completion, the reaction mixture was filtered through a celite bed, the filter pad was washed with ethyl acetate (100 mL) and the filtrate was concentrated in vacuo. The crude material was purified by combi-flash chromatography, by eluting with ethyl acetate, to afford tert-butyl 4-(2-(1-(2-(2,6-dioxopiperidin-3-yl)-1-oxoisoindolin-4-yl)piperidin-4-yl)ethyl)benzamide (Intermediate 694, 0.80 g, 89%) as a yellow solid. ¹H NMR (400 MHz, DMSO-d₆) δ ppm 1.30-1.37 (m, 2H), 1.56-1.61 (m, 2H), 1.80-1.86 (m, 2H), 2.55-2.70 (m, 4H), 2.86-2.92 (m, 1H), 3.07-3.10 (m, 6H), 3.35-2-3.42 (m, 1H), 4.26-4.31 (m, 1H), 4.41-4.45 (m, 1H), 5.10-5.12 (m, 1H), 7.14-7.16 (m, 1H), 7.27-7.30 (m, 3H), 7.40-7.44 (m, 1H), 7.79-7.81 (m, 1H), 7.86-7.90 (m, 1H), 9.11 (bs, 1H), 10.97 (s, 1H). Mass spec m/z 475.2 [M+H]⁺.

### Step 5

### Intermediate 695: tert-butyl 6-(4-(2-(1-(2-(2,6-dioxopiperidin-3-yl)-1-oxoisoindolin-4-yl)piperidin-4-yl)ethyl)benzamido)-2-((R)-1-methylpyrrolidin-2-yl)-1H-pyrrolo[3,2-c]pyridine-1-carboxylate

To a solution of tert-butyl 4-(2-(1-(2-(2,6-dioxopiperidin-3-yl)-1-oxoisoindolin-4-yl)piperidin-4-yl)ethyl)benzamide (Intermediate 694, 0.5 g, 1.0 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.5 g, 1.0 mmol) and cesium carbonate (1.0 g, 4.0 mmol). The reaction mixture was purged with argon for 15 min then BrettPhos (0.20 g, 0.20 mmol) and BrettPhos Pd G3 (0.10 g, 0.20 mmol) were added. The reaction mixture was purged with argon for a further 10 min, then heated at 100° C. for 2 h. After completion, the reaction mixture was filtered through a celite bed, the filter pad was washed with ethyl acetate (100 mL) and the filtrate was concentrated in vacuo. The crude material was purified by combi-flash chromatography, by eluting with 85% ethyl acetate in heptane, to afford tert-butyl 6-(4-(6-(2-(2,6-dioxo-1-((2-(trimethylsilyl)ethoxy)methyl)piperidin-3-yl)-1-oxoisoindolin-4-yl)hex-5-yn-1-yl)-3,5-difluorobenzamido)-2-((R)-1-methylpyrrolidin-2-yl)-1H-pyrrolo[3,2-c]pyridine-1-carboxylate (Intermediate 695, 0.40 g, 40%) as a brown solid. ¹H NMR (400 MHz, DMSO-d₆) δ ppm 1.15-1.22 (m, 4H), 1.25-1.96 (m, 12H), 1.97-2.02 (m, 1H), 2.25-2.41 (m, 5H), 2.51-2.78 (m, 6H), 2.81-2.97 (m, 1H), 3.10-3.20 (m, 2H), 3.33-2-3.51 (m, 4H), 3.92-4.08 (m, 1H), 4.28-4.31 (m, 1H), 4.42-4.46 (m, 1H), 5.09-5.13 (m, 1H), 7.15-7.17 (m, 1H), 7.28-7.42 (m, 5H), 7.79-7.89 (m, 1H), 7.98-7.80 (m, 1H), 8.59 (s, 1H), 8.93 (s, 1H), 10.59 (bs, 1H), 10.97 (s, 1H). Mass spec m/z 774.2 [M+H]⁺.

### Step 6

### Example 177: 4-(2-(1-(2-(2,6-dioxopiperidin-3-yl)-1-oxoisoindolin-4-yl)piperidin-4-yl)ethyl)-N-(2-((R)-1-methylpyrrolidin-2-yl)-1H-pyrrolo[3,2-c]pyridin-6-yl)benzamide

To a cooled (0° C.) solution of tert-butyl 6-(4-(6-(2-(2,6-dioxo-1-((2-(trimethylsilyl)ethoxy)methyl)piperidin-3-yl)-1-oxoisoindolin-4-yl)hex-5-yn-1-yl)-3,5-difluorobenzamido)-2-((R)-1-methylpyrrolidin-2-yl)-1H-pyrrolo[3,2-c]pyridine-1-carboxylate (Intermediate 695, 1 g, 1.39 mmol) in 1,4-dioxane (5 mL) was added 4M hydrochloric acid in 1,4-dioxane (5 mL) and the reaction mixture was stirred at room temperature for 16 h. After completion, the reaction mixture was concentrated in vacuo. The crude material was purified by preparative HPLC (Method A) to afford 4-(2-(1-(2-(2,6-dioxopiperidin-3-yl)-1-oxoisoindolin-4-yl)piperidin-4-yl)ethyl)-N-(2-((R)-1-methylpyrrolidin-2-yl)-1H-pyrrolo[3,2-c]pyridin-6-yl)benzamide (Example 177, 0.07 g, 20%) as an off-white solid. ¹H NMR (400 MHz, DMSO-d₆) δ ppm 1.21-1.40 (m, 4H), 1.48-1.56 (m, 2H), 1.79-2.05 (m, 7H), 2.11-2.32 (m, 4H), 2.41-2.45 (m, 1H), 2.52-2.61 (m, 1H), 2.57-2.75 (m, 4H), 2.88-2.96 (m, 1H), 3.12-3.18 (m, 1H), 3.32-2-3.42 (m, 2H), 4.27-4.31 (m, 1H), 4.41-4.45 (m, 1H), 5.08-5.13 (m, 1H), 6.41 (s, 1H), 7.16 (d, J=7.6 Hz, 1H), 7.29 (d, J=7.6 Hz, 1H), 7.35 (d, J=8.0 Hz, 2H), 7.40-7.44 (m, 1H), 7.89 (d, J=8.0 Hz, 2H), 8.19 (s, 1H), 8.51 (s, 1H), 10.37 (br s, 1H), 10.97 (br s, 1H), 11.37 (br s, 1H). Mass spec m/z 674.0 [M−H]⁻.

### Example 178 was Synthesised Following Scheme 171

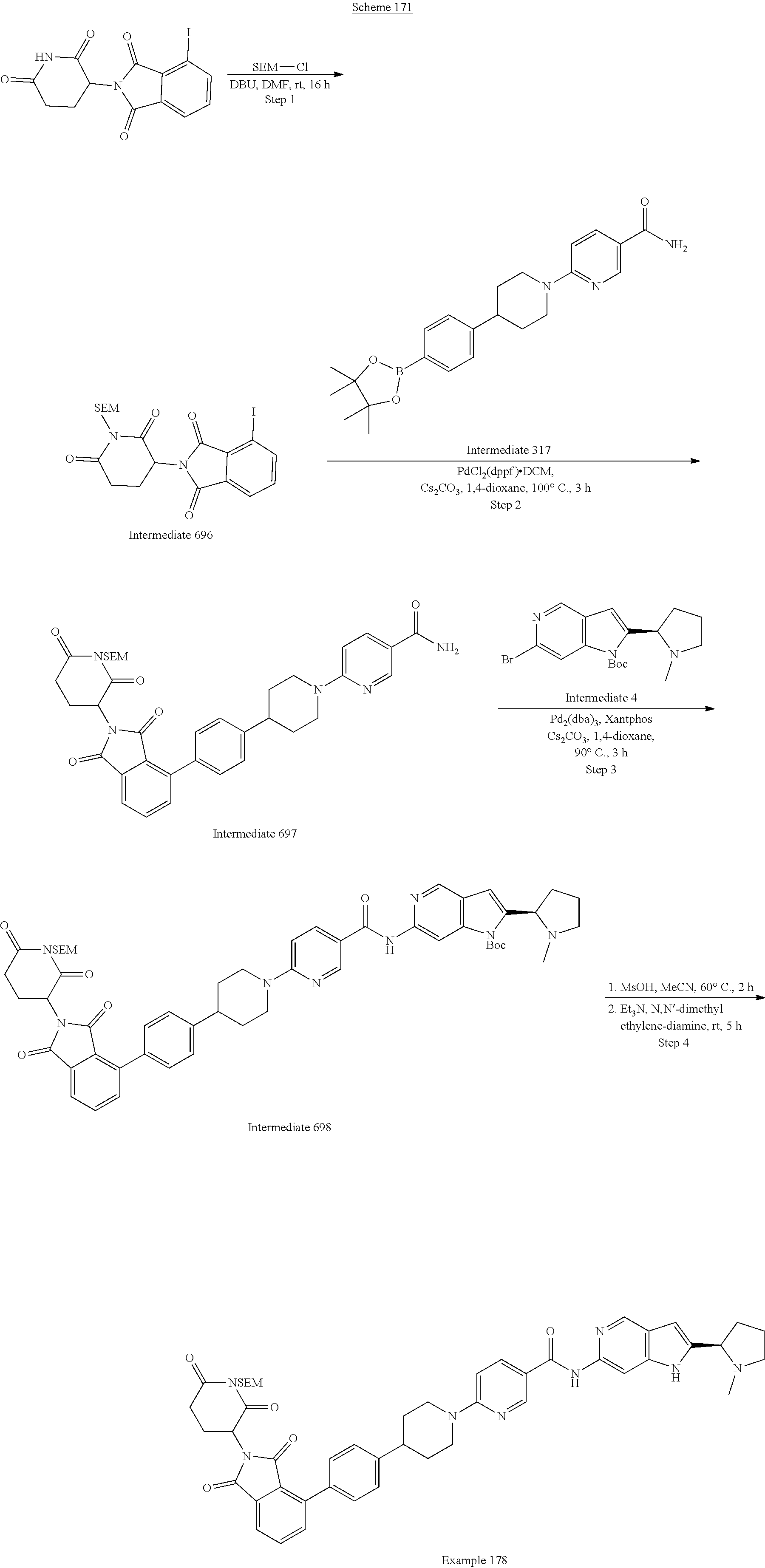

### Step 1

### Intermediate 696: 2-(2,6-dioxo-1-((2-(trimethylsilyl)ethoxy)methyl)piperidin-3-yl)-4-iodoisoindoline-1,3-dione

To a cooled (0° C.) solution of 2-(2,6-dioxopiperidin-3-yl)-4-iodoisoindoline-1,3-dione (CAS No. 959150-64-2, 1.0 g, 2.60 mmol) in dimethylformamide (6 mL) was added 1,8-diazabicyclo[5.4.0]undec-7-ene (1.0 mL, 6.50 mmol) and the reaction mixture was stirred at 0° C. for 10 min. 2-(Trimethylsilyl)ethoxymethyl chloride (1.2 mL, 6.50 mmol) was then added and the reaction mixture stirred at room temperature for 16 h. The reaction mixture was then quenched with ice-cold water (100 mL) and extracted with ethyl acetate (2×100 mL). The combined organic layers were washed with brine (100 mL), dried over anhydrous Na₂SO₄ and concentrated in vacuo. The crude material was purified by combi-flash chromatography, by eluting with 15% ethyl acetate in heptane, to afford 2-(2,6-dioxo-1-((2-(trimethylsilyl)ethoxy)methyl)piperidin-3-yl)-4-iodoisoindoline-1,3-dione (Intermediate 696, 0.90 g, 90%) as a colourless liquid. ¹H NMR (400 MHz, DMSO-d₆) δ ppm −0.01 (s, 9H), 0.82-0.89 (m, 2H), 2.07-2.14 (m, 1H), 2.54-2.56 (m, 1H), 2.77-2.85 (m, 1H), 2.98-3.09 (m, 1H), 3.46-3.59 (m, 2H), 5.09 (s, 2H), 5.28-5.32 (m, 1H), 7.60 (t, J=7.70 Hz, 1H), 7.94 (d, J=7.09 Hz, 1H), 8.26-8.31 (m, 1H).

### Step 2

### Intermediate 697: 6-(4-(4-(2-(2,6-dioxo-1-((2-(trimethylsilyl)ethoxy)methyl)piperidin-3-yl)-1,3-dioxoisoindolin-4-yl)phenyl)piperidin-1-yl)nicotinamide

To a solution of 6-(4-(4-(4,4,5,5-tetramethyl-1,3,2-dioxaborolan-2-yl)phenyl)piperidin-1-yl)nicotinamide (Comp-Intermediate 317, 1.9 g, 4.7 mmol) in 1,4-dioxane (20 mL) was added 2-(2,6-dioxo-1-((2-(trimethylsilyl)ethoxy)methyl)piperidin-3-yl)-4-iodoisoindoline-1,3-dione (Intermediate 696, 1.5 g, 2.9 mmol) followed by cesium carbonate (2.9 g, 8.7 mmol). The reaction mixture was purged with argon for 15 min then PdCl₂(dppf). CH₂Cl₂ (0.24 g, 0.29 mmol) was added, and the reaction mixture was heated at 100° C. for 3 h. After completion, the reaction mixture was concentrated in vacuo. The crude material was purified by combi-flash chromatography, by eluting with 5% MeOH in DCM, to afford 6-(4-(4-(2-(2,6-dioxo-1-((2-(trimethylsilyl)ethoxy)methyl)piperidin-3-yl)-1,3-dioxoisoindolin-4-yl)phenyl)piperidin-1-yl)nicotinamide (Intermediate 697, 1.0 g, 51%) as a yellow solid. Mass spec m/z 668.2 [M+H]⁺.

### Step 3

### Intermediate 698: tert-butyl 6-(6-(4-(4-(2-(2,6-dioxo-1-((2-(trimethylsilyl)ethoxy)methyl)piperidin-3-yl)-1,3-dioxoisoindolin-4-yl)phenyl)piperidin-1-yl)nicotinamido)-2-((R)-1-methylpyrrolidin-2-yl)-1H-pyrrolo[3,2-c]pyridine-1-carboxylate

To a solution of 6-(4-(4-(2-(2,6-dioxo-1-((2-(trimethylsilyl)ethoxy)methyl)piperidin-3-yl)-1,3-dioxoisoindolin-4-yl)phenyl)piperidin-1-yl)nicotinamide (Intermediate 697, 0.50 g, 0.74 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.31 g, 0.82 mmol) followed by cesium carbonate (0.73 g, 2.24 mmol). The reaction mixture was purged with argon for 15 min, then Pd₂(dba)₃ (0.10 g, 0.11 mmol) and Xantphos (0.13 g, 0.22 mmol) were added. The reaction mixture was further purged with argon for 10 min and heated at 90° C. for 3 h. The reaction mixture was concentrated in vacuo and the crude material purified by combi-flash chromatography, by eluting with 15% MeOH in DCM, to afford tert-butyl 6-(6-(4-(4-(2-(2,6-dioxo-1-((2-(trimethylsilyl)ethoxy)methyl)piperidin-3-yl)-1,3-dioxoisoindolin-4-yl)phenyl)piperidin-1-yl)nicotinamido)-2-((R)-1-methylpyrrolidin-2-yl)-1H-pyrrolo[3,2-c]pyridine-1-carboxylate (Intermediate 698, 0.50 g, 69%) as an off-white solid. Mass spec m/z 967.5 [M+H]⁺.

### Step 4

### Example 178: 6-(4-(4-(2-(2,6-dioxopiperidin-3-yl)-1,3-dioxoisoindolin-4-yl)phenyl)piperidin-1-yl)-N-(2-((R)-1-methylpyrrolidin-2-yl)-1H-pyrrolo[3,2-c]pyridin-6-yl)nicotinamide

To a solution of tert-butyl 6-(6-(4-(4-(2-(2,6-dioxo-1-((2-(trimethylsilyl)ethoxy)methyl)piperidin-3-yl)-1,3-dioxoisoindolin-4-yl)phenyl)piperidin-1-yl)nicotinamido)-2-((R)-1-methylpyrrolidin-2-yl)-1H-pyrrolo[3,2-c]pyridine-1-carboxylate (Intermediate 698, 0.55 g, 0.56 mmol) in acetonitrile (10 mL) was added methanesulfonic acid (0.37 mL, 5.68 mmol) and the reaction mixture was heated at 50° C. for 2 h. The reaction mixture was cooled to room temperature, then N,N′-dimethylethylenediamine (0.68 mL, 5.68 mmol) followed by triethylamine (1.59 mL, 11.37 mmol) were added and the mixture was stirred at room temperature for 5 h. The reaction mixture was poured into ice cold water (100 mL), the resultant precipitate was collected by filtration and dried in vacuo. The crude solid was purified by preparative HPLC (Method A) to afford 6-(4-(4-(2-(2,6-dioxopiperidin-3-yl)-1,3-dioxoisoindolin-4-yl)phenyl)piperidin-1-yl)-N-(2-((R)-1-methylpyrrolidin-2-yl)-1H-pyrrolo[3,2-c]pyridin-6-yl)nicotinamide (Example 178, 0.04 g, 10%) as an off-white solid. ¹H NMR (400 MHz, DMSO-d₆) δ ppm 1.72-1.80 (m, 2H), 1.84-1.96 (m, 5H), 2.01-2.07 (m, 1H), 2.10-2.15 (m, 1H), 2.16 (s, 3H), 2.22-2.28 (m, 1H), 2.55-2.63 (m, 2H), 2.81-3.08 (m, 4H), 3.12-3.15 (m, 1H), 3.28-3.31 (m, 1H), 4.62-4.68 (m, 2H), 5.08-5.14 (m, 1H), 6.38 (s, 1H), 6.95 (d, J=9.20 Hz, 1H), 7.38 (d, J=8.40 Hz, 2H), 7.54 (d, J=8.40 Hz, 2H), 7.77-7.84 (m, 1H), 7.86-7.94 (m, 2H), 8.15-8.21 (m, 2H), 8.48 (s, 1H), 8.83 (d, J=2.40 Hz, 1H), 10.28 (br s, 1H), 11.10 (br s, 1H), 11.34 (br s, 1H). Mass spec m/z 737.4 [M+H]⁺.

### Example 179 was Synthesised Following Scheme 172

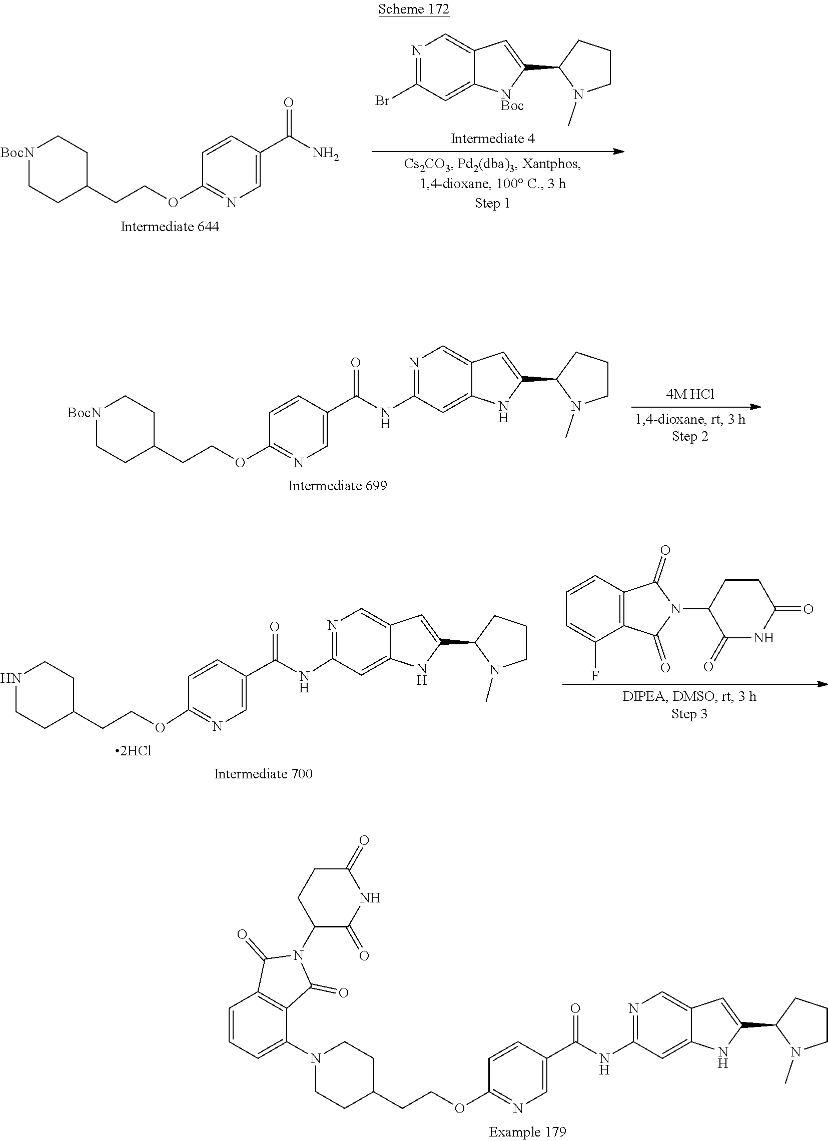

### Step 1

### Intermediate 699: tert-butyl (R)-6-(6-(2-(1-(tert-butoxycarbonyl)piperidin-4-yl)ethoxy)nicotinamido)-2-(1-methylpyrrolidin-2-yl)-1H-pyrrolo[3,2-c]pyridine-1-carboxylate

To a solution of tert-butyl 4-(2-((5-carbamoylpyridin-2-yl)oxy)ethyl)piperidine-1-carboxylate (Intermediate 644, 2.0 g, 5.72 mmol) in 1,4-dioxane (20 mL) were added tert-butyl (R)-6-bromo-2-(1-methylpyrrolidin-2-yl)-1H-pyrrolo[3,2-c]pyridine-1-carboxylate (Intermediate 4, 2.39 g, 6.29 mmol) followed by cesium carbonate (3.31 g, 17.2 mmol). The reaction mixture was purged with argon for 15 min then Pd₂(dba)₃ (0.82 g, 0.85 mmol) and Xantphos (1.02 g, 1.71 mmol) were added. The reaction mixture was further purged with argon for 10 min and heated at 100° C. for 3 h. The reaction mixture was then filtered through a celite bed, the filter pad was washed with ethyl acetate (100 mL) and the filtrate concentrated in vacuo. The crude material was purified by combi-flash chromatography, by eluting with 10% methanol in dichloromethane, to afford tert-butyl (R)-6-(6-(2-(1-(tert-butoxycarbonyl)piperidin-4-yl)ethoxy)nicotinamido)-2-(1-methylpyrrolidin-2-yl)-1H-pyrrolo[3,2-c]pyridine-1-carboxylate (Intermediate 699, 1.8 g, 23%) as an off-white solid. ¹H NMR (400 MHz, DMSO-d₆) δ ppm 0.98-1.10 (m, 3H), 1.39 (s, 9H), 1.20-1.31 (m, 4H), 1.57-1.69 (m, 13H), 2.30-2.39 (m, 5H), 2.63-2.75 (m, 2H), 3.89-3.95 (m, 3H), 4.36-4.41 (m, 2H), 6.75 (s, 1H), 6.88 (d, J=8.40 Hz, 1H), 8.28 (d, J=8.40 Hz, 1H), 8.59 (s, 1H), 8.84 (s, 1H), 8.91 (s, 1H), 10.79 (s, 1H). Mass spec m/z 649.3 [M+H]⁺.

### Step 2

### Intermediate 700: (R)—N-(2-(1-methylpyrrolidin-2-yl)-1H-pyrrolo[3,2-c]pyridin-6-yl)-6-(2-(piperidin-4-yl)ethoxy)nicotinamide hydrochloride

To a cooled (0° C.) solution of tert-butyl (R)-6-(6-(2-(1-(tert-butoxycarbonyl)piperidin-4-yl)ethoxy)nicotinamido)-2-(1-methylpyrrolidin-2-yl)-1H-pyrrolo[3,2-c]pyridine-1-carboxylate (Intermediate 699, 1.80 g, 2.80 mmol) in dichloromethane (10 mL) was added 4M hydrochloric acid in 1,4-dioxane (15 mL) and the reaction mixture was stirred at room temperature for 3 h. The reaction mixture was then concentrated in vacuo to afford (R)—N-(2-(1-methylpyrrolidin-2-yl)-1H-pyrrolo[3,2-c]pyridin-6-yl)-6-(2-(piperidin-4-yl)ethoxy)nicotinamide hydrochloride (Intermediate 700, 1.20 g, 96%) as a white solid, which was used for the next step without further purification. Mass spec m/z 449.1 [M+H]⁺.

### Step 3

### Example 179: 6-(2-(1-(2-(2,6-dioxopiperidin-3-yl)-1,3-dioxoisoindolin-4-yl)piperidin-4-yl)ethoxy)-N-(2-((R)-1-methylpyrrolidin-2-yl)-1H-pyrrolo[3,2-c]pyridin-6-yl)nicotinamide

To a solution of (R)—N-(2-(1-methylpyrrolidin-2-yl)-1H-pyrrolo[3,2-c]pyridin-6-yl)-6-(2-(piperidin-4-yl)ethoxy)nicotinamide hydrochloride (Intermediate 700, 1.20 g, 2.70 mmol) in dimethyl sulfoxide (15 mL) was added N,N-diisopropylethylamine (1.9 mL, 11.0 mmol) followed by 2-(2,6-dioxopiperidin-3-yl)-4-fluoroisoindoline-1,3-dione (CAS No. 835616-60-9, 0.66 g, 2.40 mmol) and the reaction mixture was heated at 80° C. for 3 h. The reaction mixture was then concentrated in vacuo and the residue triturated with diethyl ether (2×10 mL). The crude material was purified by preparative HPLC (Method A) to afford 6-(2-(1-(2-(2,6-dioxopiperidin-3-yl)-1,3-dioxoisoindolin-4-yl)piperidin-4-yl)ethoxy)-N-(2-((R)-1-methylpyrrolidin-2-yl)-1H-pyrrolo[3,2-c]pyridin-6-yl)nicotinamide (Example 179, 0.08 g, 4%) as a white solid. ¹H NMR (400 MHz, DMSO-d₆) δ ppm 1.21-1.32 (m, 2H), 1.69-1.77 (m, 2H), 1.78-1.90 (m, 5H), 2.01-2.08 (m, 1H), 2.17 (s, 3H), 2.24-2.32 (m, 1H), 2.45-2.49 (m, 1H), 2.63-2.67 (m, 1H), 2.81-2.88 (m, 1H), 2.96-3.04 (m, 2H), 3.10-3.16 (m, 1H), 3.33-3.37 (m, 1H), 3.91-3.97 (m, 2H), 4.00-4.08 (m, 2H), 4.39-4.44 (m, 2H), 5.00-5.08 (m, 1H), 6.46 (s, 1H), 6.93 (d, J=8.68 Hz, 1H), 7.25 (d, J=9.78 Hz, 1H), 7.30 (br s, 1H), 7.67 (d, J=8.68 Hz, 1H), 8.13 (s, 1H), 8.25-8.31 (m, 1H), 8.53 (s, 1H), 8.81 (s, 1H). Mass spec m/z 705.2 [M+H]⁺.

### Example 180 was Synthesised Following Scheme 173

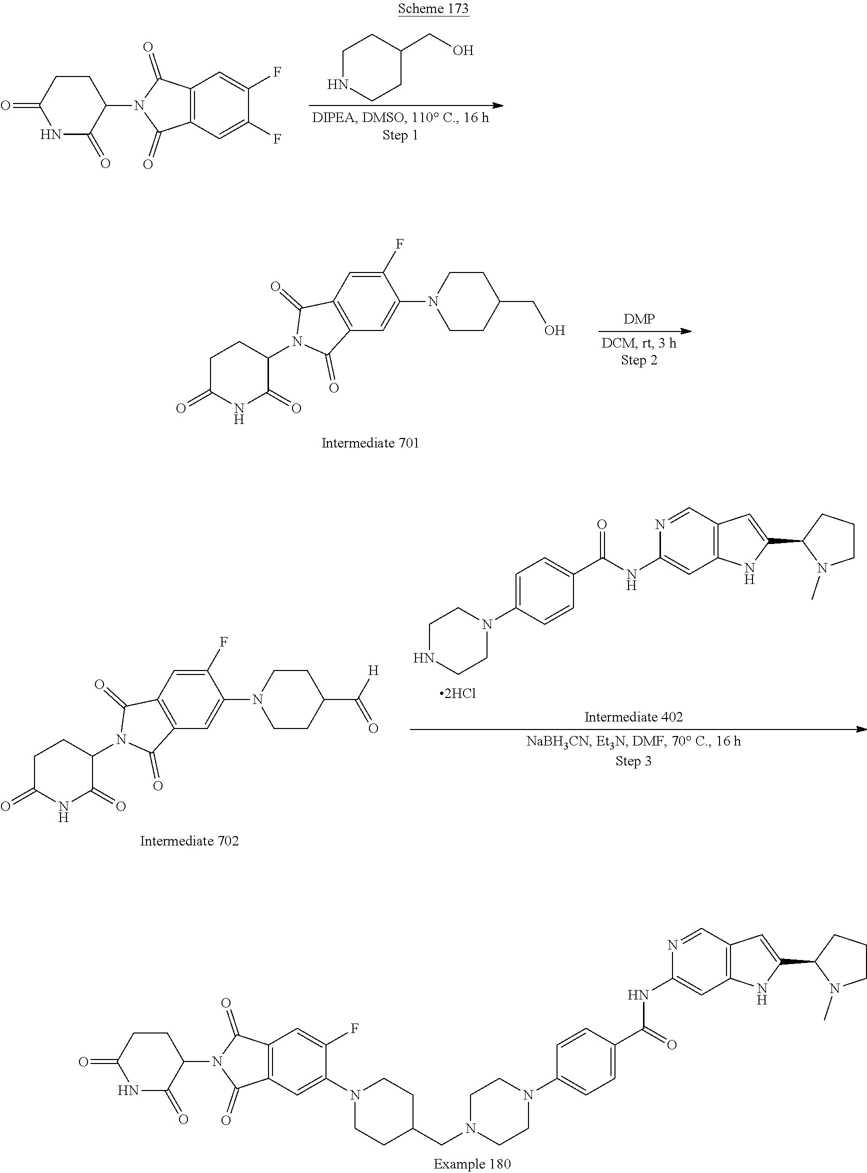 [SMILES: O=C1CCC(N2C(=O)c3cc(F)c(F)cc3C2=O)C(=O)N1 ; OCC1CCNCC1 ; O=C1CCC(N2C(=O)c3cc(F)c(N4CCC(CO)CC4)cc3C2=O)C(=O)N1 ; O=CC1CCN(c2cc3c(cc2F)C(=O)N(C2CCC(=O)NC2=O)C3=O)CC1 ; CN1CCC[C@@H]1c1cc2cnc(NC(=O)c3ccc(N4CCNCC4)cc3)cc2[nH]1 ; CN1CCC[C@@H]1c1cc2cnc(NC(=O)c3ccc(N4CCN(CC5CCN(c6cc7c(cc6F)C(=O)N(C6CCC(=O)NC6=O)C7=O)CC5)CC4)cc3)cc2[nH]1]

### Step 1

### Intermediate 701: 2-(2,6-dioxopiperidin-3-yl)-5-fluoro-6-(4-(hydroxymethyl)piperidin-1-yl)isoindoline-1,3-dione

To a solution of 2-(2,6-dioxopiperidin-3-yl)-5,6-difluoroisoindoline-1,3-dione (CAS No. 1496997-41-1, 0.70 g, 2.38 mmol) and piperidin-4-ylmethanol (CAS No. 6457-49-4, 0.30 g, 2.62 mmol) in dimethyl sulfoxide (5 mL) was added N,N-diisopropylethylamine amine (0.83 mL, 4.76 mmol). The reaction mixture was heated at 110° C. for 16 h. After completion, the reaction mixture was diluted with water (200 mL) and extracted with ethyl acetate (2×50 mL). The combined organic layers were washed water (2×50 mL), dried over Na₂SO₄ and concentrated in vacuo to afford 2-(2,6-dioxopiperidin-3-yl)-5-fluoro-6-(4-(hydroxymethyl)piperidin-1-yl)isoindoline-1,3-dione (Intermediate 701, 0.70 g, 76%) as a yellow solid which was used into next step without purification. Mass spec m/z 390.2 [M+H]⁺.

### Step 2

### Intermediate 702: 1-(2-(2,6-dioxopiperidin-3-yl)-6-fluoro-1,3-dioxoisoindolin-5-yl)piperidine-4-carbaldehyde

To a cooled (0° C.) solution of 2-(2,6-dioxopiperidin-3-yl)-5-fluoro-6-(4-(hydroxymethyl)piperidin-1-yl)isoindoline-1,3-dione (Intermediate 701, 0.70 g, 1.80 mmol) in DCM (20 mL) was added Dess-Martin periodinane (1.53 g, 3.60 mmol) and the reaction mixture was stirred at room temperature for 3 h. After completion, the reaction mixture was diluted with water (100 mL) and extracted with ethyl acetate (2×100 mL). The combined organic layers were washed with saturated aqueous sodium bicarbonate solution (50 mL) and saturated aqueous sodium thiosulphate solution (50 mL). The combined organic layers were dried over Na₂SO₄ and concentrated in vacuo to afford 1-(2-(2,6-dioxopiperidin-3-yl)-6-fluoro-1,3-dioxoisoindolin-5-yl)piperidine-4-carbaldehyde (Intermediate 702, 0.38 g) as a yellow solid which was carried forward into next step without purification. The desired product was confirmed by LCMS. Mass spec m/z 388.2 [M+H]⁺.

### Step 3

### Example 180: 4-(4-((1-(2-(2,6-dioxopiperidin-3-yl)-6-fluoro-1,3-dioxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide

To a solution of 1-(2-(2,6-dioxopiperidin-3-yl)-6-fluoro-1,3-dioxoisoindolin-5-yl)piperidine-4-carbaldehyde (Intermediate 702, 0.35 g, 0.90 mmol) in dimethylformamide (5 mL) was added a solution of (R)—N-(2-(1-methylpyrrolidin-2-yl)-1H-pyrrolo[3,2-c]pyridin-6-yl)-4-(piperazin-1-yl)benzamide dihydrochloride (Intermediate 402, 0.44 g, 0.99 mmol) in dimethylformamide (5 mL) followed by triethylamine (0.51 mL, 3.61 mmol) and sodium cyanoborohydride (0.17 g, 2.71 mmol) and the reaction mixture was heated at 70° C. for 16 h. After completion, the reaction mixture was quenched with ice cold water (20 mL), the resultant precipitate was collected by filtration and dried in vacuo. The crude material was purified by combi-flash chromatography, by eluting with 50% isopropanol in DCM, to afford 4-(4-((1-(2-(2,6-dioxopiperidin-3-yl)-6-fluoro-1,3-dioxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide (Example 180, 0.068 g, 10%) as a pale yellow solid. H NMR (400 MHz, DMSO-d₆) δ ppm 1.22-1.33 (m, 3H), 1.78-1.91 (m, 6H), 2.02-2.05 (m, 1H), 2.12-2.14 (m, 1H), 2.16 (m, 3H), 2.26-2.33 (m, 3H), 2.51-2.57 (m, 4H), 2.62-2.68 (m, 2H), 2.84-2.94 (m, 3H), 3.12-3.16 (m, 1H), 3.29-3.32 (m, 4H), 3.61-3.63 (m, 2H), 5.08-5.13 (m, 1H), 6.37 (s, 1H), 6.98 (d, J=9.20 Hz, 2H), 7.45 (d, J=7.20 Hz, 1H), 7.71 (d, J=11.60 Hz, 1H), 7.96 (d, J=8.80 Hz, 2H), 8.17 (s, 1H), 8.48 (s, 1H), 10.10 (s, 1H), 11.10 (s, 1H), 11.31 (s, 1H). Mass spec m/z 776.0 [M+H]⁺.

### Example 181 was Synthesised Following Scheme 174

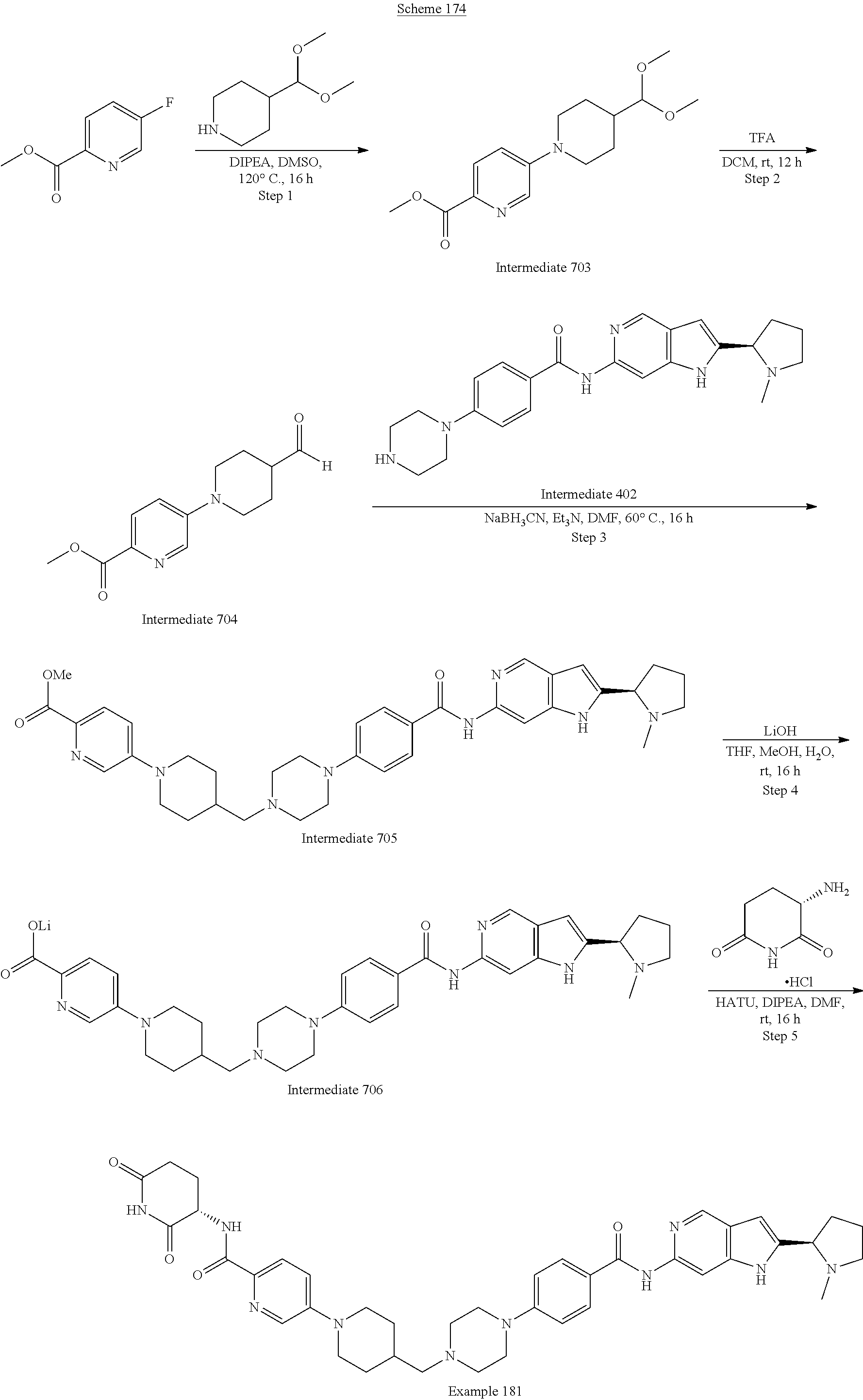

### Step 1

### Intermediate 703: methyl 5-(4-(dimethoxymethyl)piperidin-1-yl)picolinate

To a solution of methyl 5-fluoropicolinate (CAS No. 107504-07-4, 5.00 g, 32.23 mmol) and 4-(dimethoxymethyl)piperidine (CAS No. 188646-83-5, 5.65 g, 35.45 mmol) in dimethyl sulfoxide (20 mL) was added N,N-diisopropylethylamine (22.0 mL, 128.9 mmol) and the reaction mixture was heated at 120° C. for 16 h. After completion, the reaction mixture was diluted with water (100 mL), the resultant precipitate was collected by filtration, washed with n-pentane (20 mL), and dried in vacuo to afford methyl 5-(4-(dimethoxymethyl)piperidin-1-yl)picolinate (Intermediate 703, 8.5 g, 90%) as an off-white solid. ¹H NMR (400 MHz, DMSO-d) δ ppm 1.25-1.31 (m, 2H), 1.65-1.75 (m, 2H), 1.82-1.85 (m, 1H), 2.81-2.87 (m, 2H), 3.26 (s, 6H, 3.79 (s, 3H), 3.96-4.00 (m, 2H), 4.06-4.08 (m, 1H), 7.31 (d, J=8.8 Hz, 1H), 7.84 (d, J=8.80 Hz, 1H), 8.35 (s, 1H). Mass spec m/z 295.1 [M+H]⁺.

### Step 2

### Intermediate 704: methyl 5-(4-formylpiperidin-1-yl)picolinate

To a cooled (0° C.) solution of methyl 5-(4-(dimethoxymethyl)piperidin-1-yl)picolinate (Intermediate 703, 1.0 g, 3.39 mmol) in DCM (50 mL) was added trifluoroacetic acid (5.81 g, 50.95 mmol) and the reaction mixture was stirred at room temperature for 12 h. After completion, the reaction mixture was concentrated in vacuo to afford methyl 5-(4-formylpiperidin-1-yl)picolinate (Intermediate 704, 1.2 g) as a pale-yellow oil which was carried forward into next step without purification. ¹H NMR (400 MHz, DMSO-d₆) δ ppm 1.49-1.62 (m, 2H), 1.91-1.98 (m, 2H), 2.59-2.62 (m, 1H), 3.06-3.15 (m, 2H), 3.81 (s, 3H), 3.84-3.91 (m, 2H), 7.41 (dd, J=8.8, 2.80 Hz, 1H), 7.88 (d, J=8.40 Hz, 1H), 8.37 (d, J=2.40 Hz, 1H), 9.62 (br s, 1H). Mass spec m/z 249.1 [M+H]⁺.

### Step 3

### Intermediate 705: methyl (R)-5-(4-((4-(4-((2-(1-methylpyrrolidin-2-yl)-1H-pyrrolo[3,2-c]pyridin-6-yl)carbamoyl)phenyl)piperazin-1-yl)methyl)piperidin-1-yl)picolinate

To a solution of methyl 5-(4-formylpiperidin-1-yl)picolinate (Intermediate 704, 0.70 g, 2.82 mmol) in dimethylformamide (10 mL) was added a solution of (R)—N-(2-(1-methylpyrrolidin-2-yl)-1H-pyrrolo[3,2-c]pyridin-6-yl)-4-(piperazin-1-yl)benzamide dihydrochloride (Intermediate 402, 3.73 g, 8.46 mmol) in dimethylformamide (5 mL) followed by triethylamine (1.14 g, 11.28 mmol) and sodium cyanoborohydride (0.53 g, 8.46 mmol). The resultant reaction mixture was heated at 60° C. for 16 h, then quenched with ice cold water (100 mL). The resultant precipitate was collected by filtration, washed with n-pentane (10 mL), and dried in vacuo to afford methyl (R)-5-(4-((4-(4-((2-(1-methylpyrrolidin-2-yl)-1H-pyrrolo[3,2-c]pyridin-6-yl)carbamoyl)phenyl)piperazin-1-yl)methyl)piperidin-1-yl)picolinate (Intermediate 705, 1.10 g) as an off-white solid which was carried forward into next step without purification. ¹H NMR (400 MHz, DMSO-d₆) δ ppm 1.09-1.28 (m, 3H), 1.82-1.95 (m, 8H), 2.17 (s, 3H), 2.19-2.22 (m, 3H), 2.85-2.97 (m, 4H), 3.11-3.21 (m, 2H), 3.23-3.29 (m, 4H), 3.81 (s, 3H), 3.95-4.33 (m, 2H), 6.39 (s, 1H), 6.99 (d, J=8.40 Hz, 2H), 7.33 (d, J=8.0 Hz, 1H), 7.86 (d, J=8.80 Hz, 1H), 7.97 (d, J=8.40 Hz, 2H), 8.19 (s, 1H), 8.38 (s, 1H), 8.49 (s, 1H), 10.10 (s, 1H), 11.34 (s, 1H). Mass spec m/z 637.4 [M+H]⁺.

### Step 4

### Intermediate 706: Lithium salt of (R)-5-(4-((4-(4-((2-(1-methylpyrrolidin-2-yl)-1H-pyrrolo[3,2-c]pyridin-6-yl)carbamoyl)phenyl)piperazin-1-yl)methyl)piperidin-1-yl)picolinic acid

To a solution of methyl (R)-5-(4-((4-(4-((2-(1-methylpyrrolidin-2-yl)-1H-pyrrolo[3,2-c]pyridin-6-yl)carbamoyl)phenyl)piperazin-1-yl)methyl)piperidin-1-yl)picolinate (Intermediate 705, 1.10 g, 1.17 mmol) in tetrahydrofuran (4.0 mL), methanol (4.0 mL) and water (2.0 mL) was added lithium hydroxide (0.29 g, 6.90 mmol) and the reaction mixture was stirred at room temperature for 16 h. The reaction mixture was concentrated in vacuo and the resulting crude residue was triturated with n-pentane and dried in vacuo to afford the lithium salt of (R)-5-(4-((4-(4-((2-(1-methylpyrrolidin-2-yl)-1H-pyrrolo[3,2-c]pyridin-6-yl)carbamoyl)phenyl)piperazin-1-yl)methyl)piperidin-1-yl)picolinic acid (Intermediate 706, 0.9 g, 80%) as an off-white solid which was used as such into next step without purification. ¹H NMR (400 MHz, DMSO-d₆) δ ppm 1.10-1.31 (m, 3H), 1.72-1.89 (m, 5H), 2.16 (s, 3H), 2.19-2.26 (m, 2H), 2.28-2.34 (m, 2H), 2.72-2.83 (m, 2H), 3.11-3.19 (m, 2H), 3.21-3.29 (m, 8H), 3.78-3.83 (m, 2H), 6.32 (s, 1H), 6.90-7.01 (m, 2H), 7.12-7.19 (m, 1H), 7.21-7.28 (m, J=8.80 Hz, 1H), 7.36 (d, J=7.60 Hz, 1H), 7.70-7.81 (m, 2H), 7.97 (d, J=8.0 Hz, 2H), 8.05 (s, 1H), 8.41 (s, 1H), 10.04 (br s, 1H).

### Step 5

### Example 181: N—((S)-2,6-dioxopiperidin-3-yl)-5-(4-((4-(4-((2-((R)-1-methylpyrrolidin-2-yl)-1H-pyrrolo[3,2-c]pyridin-6-yl)carbamoyl)phenyl)piperazin-1-yl)methyl)piperidin-1-yl)picolinamide

To a solution of the lithium salt of (R)-5-(4-((4-(4-((2-(1-methylpyrrolidin-2-yl)-1H-pyrrolo[3,2-c]pyridin-6-yl)carbamoyl)phenyl)piperazin-1-yl)methyl)piperidin-1-yl)picolinic acid (Intermediate 706, 0.4 g, 0.63 mmol) in dimethylformamide (7.0 mL) was added N,N-diisopropylethylamine (0.44 mL, 2.54 mmol) and the mixture was stirred at room temperature for 5 min. The reaction mixture was cooled to 0° C. for the addition of HATU (0.36 g, 0.95 mmol) and (S)-3-aminopiperidine-2,6-dione hydrochloride (CAS No. 25181-50-4, 0.16 g, 0.95 mmol), then the resulting reaction mixture was stirred at room temperature for 16 h. The reaction mixture was quenched with water (100 mL), the resultant precipitate was collected by filtration and dried in vacuo. The crude material was purified by preparative HPLC (Method A) to afford N—((S)-2,6-dioxopiperidin-3-yl)-5-(4-((4-(4-((2-((R)-1-methylpyrrolidin-2-yl)-1H-pyrrolo[3,2-c]pyridin-6-yl)carbamoyl)phenyl)piperazin-1-yl)methyl)piperidin-1-yl)picolinamide (Example 181, 0.096 g, 21%) an off-white solid. ¹H NMR (400 MHz, DMSO-d₆) δ ppm 1.13-1.27 (m, 2H), 1.72-1.89 (m, 5H), 1.93-2.03 (m, 2H), 2.17 (s, 3H), 2.19-2.29 (m, 5H), 2.44-2.48 (m, 4H), 2.51-2.59 (m, 1H), 2.72-2.92 (m, 3H), 3.10-3.17 (m, 1H), 3.22-3.29 (m, 5H), 3.91-3.96 (m, 2H), 4.69-4.78 (m, 1H), 6.36 (s, 1H), 6.96 (d, J=8.80 Hz, 2H), 7.37-7.40 (m, 1H), 7.82 (d, J=8.80, 1H), 7.94 (d, J=8.80 Hz, 2H), 8.15 (s, 1H), 8.28 (d, J=2.80 Hz, 1H), 8.46 (s, 1H), 8.68 (d, J=8.40 Hz, 1H), 10.07 (s, 1H), 10.82 (s, 1H), 11.30 (br s, 1H). Mass spec m/z 733.1 [M+H]⁺.

### Example 182 was Synthesised Following Scheme 175

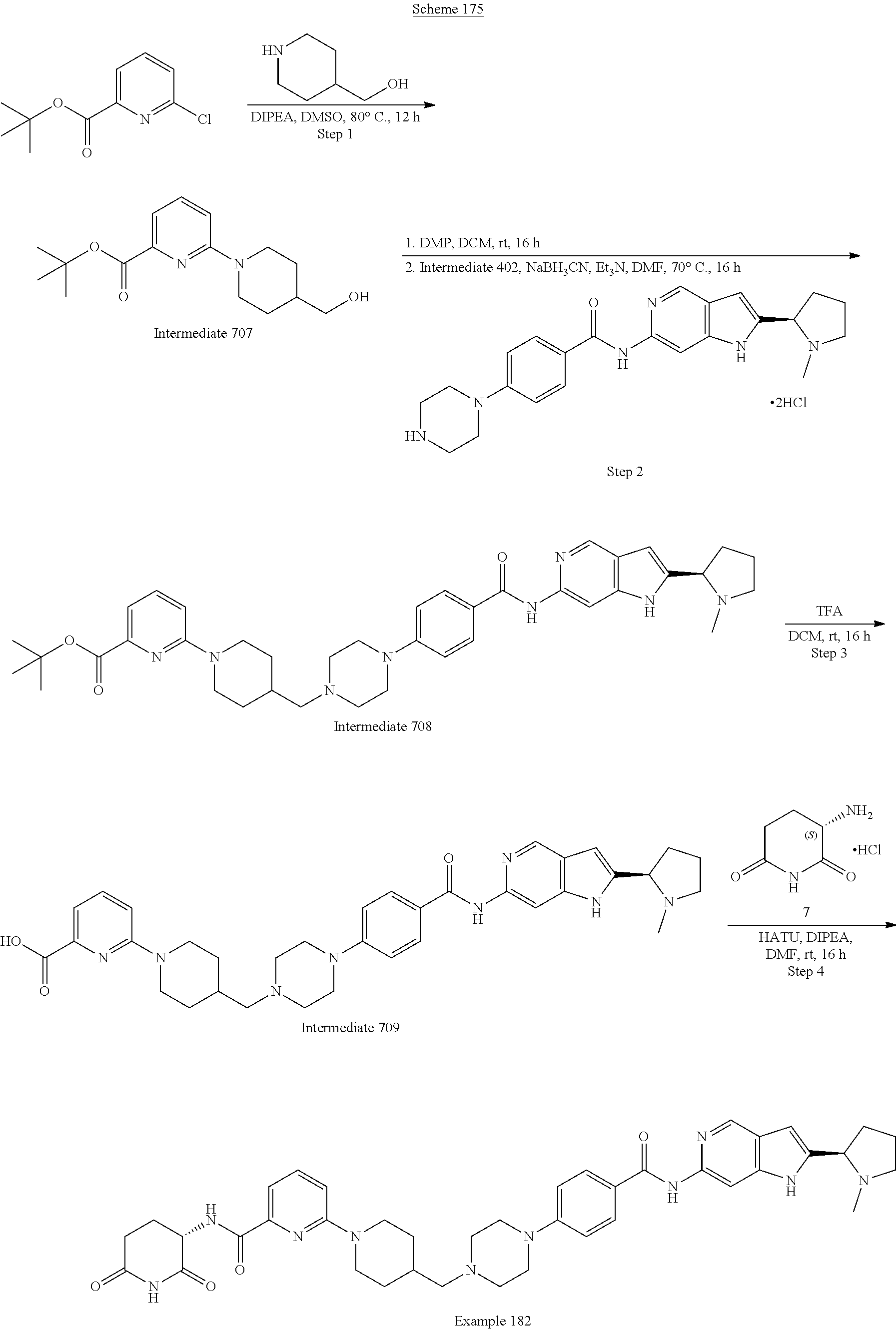

### Step 1

### Intermediate 707: tert-butyl 6-(4-(hydroxymethyl)piperidin-1-yl)picolinate

To a solution of tert-butyl 6-chloropicolinate (CAS No. 1280786-59-5, 2.0 g, 9.36 mmol) in dimethyl sulfoxide (20 mL) was added piperidin-4-ylmethanol (CAS No. 6457-49-4, 1.29 g, 11.23 mmol) followed by N,N-diisopropylethylamine (3.70 g, 28.08 mmol) and the reaction mixture was heated at 80° C. for 12 h. After completion, the reaction mixture was diluted with water (150 mL) and extracted with DCM (2×150 mL). The combined organic layers were washed with water (150 mL), dried over anhydrous Na₂SO₄ and concentrated in vacuo. The crude material was purified by combi-flash chromatography, by eluting with 70% ethyl acetate in heptane, to afford tert-butyl 6-(4-(hydroxymethyl)piperidin-1-yl)picolinate (Intermediate 707, 2.10 g, 77%) as a yellow solid. ¹H NMR (400 MHz, DMSO-d₆) δ ppm 1.03-1.19 (m, 2H), 1.52 (s, 9H), 1.55-1.63 (m, 1H), 1.67-1-77 (in, 2H), 2.72-2.83 (m, 2H), 3.21-3.27 (m, 2H), 4.31-4.39 (m, 2H), 4.42-4.48 (m, 1H), 7.00 (d, J=8.0 Hz, 1H), 7.17 (d, J=8.40 Hz, 1H), 7.60 (t, J=8.0 Hz, 1H). Mass spec m/z 293.0 [M+H]⁺.

### Step 2

### Intermediate 708: tert-butyl (R)-6-(4-((4-(4-((2-(1-methylpyrrolidin-2-yl)-1H-pyrrolo[3,2-c]pyridin-6-yl)carbamoyl)phenyl)piperazin-1-yl)methyl)piperidin-1-yl)picolinate

To a solution of tert-butyl 6-(4-(hydroxymethyl)piperidin-1-yl)picolinate (Intermediate 707, 0.50 g, 1.77 mmol) in DCM (20 mL) was added Dess-Martin periodinane (1.47 g, 3.42 mmol). The reaction mixture was stirred at room temperature for 16 h. After completion, the reaction mixture was quenched with water (120 mL) and diluted with DCM (50 mL). The organic layer was separated, washed with water (50 mL), dried over Na₂SO₄ and concentrated in vacuo to afford tert-butyl 6-(4-formylpiperidin-1-yl)picolinate (0.89 g) as a pale-yellow oil. To a mixture of the tert-butyl 6-(4-formylpiperidin-1-yl)picolinate (0.62 g, 2.13 mmol) and (R)—N-(2-(1-methylpyrrolidin-2-yl)-1H-pyrrolo[3,2-c]pyridin-6-yl)-4-(piperazin-1-yl)benzamide dihydrochloride (Intermediate 402, 0.94 g, 2.13 mmol) in dimethylformamide was added triethylamine (1.20 mL, 8.54 mmol) followed by sodium cyanoborohydride (0.40 g, 6.41 mmol) and the reaction mixture was heated at 70° C. for 16 h. After completion, the reaction mixture was diluted with water (100 mL) and extracted with ether (3×120 mL). The combined organic layers were dried over Na₂SO₄, filtered and concentrated in vacuo. The crude material was purified by combi-flash chromatography, by eluting with 78% ethyl acetate in heptane, to afford tert-butyl (R)-6-(4-((4-(4-((2-(1-methylpyrrolidin-2-yl)-1H-pyrrolo[3,2-c]pyridin-6-yl)carbamoyl)phenyl)piperazin-1-yl)methyl)piperidin-1-yl)picolinate (Intermediate 708, 0.75 g, 52%) as a pale yellow oil. ¹H NMR (400 MHz, DMSO-d₆) δ ppm 1.09-1.14 (m, 4H), 1.52 (s, 9H), 1.65-1.72 (m, 2H), 1.77-1.89 (m, 5H), 2.16 (s, 3H), 2.19-2.38 (m, 3H), 2.73-2.84 (m, 4H), 3.11-3.19 (m, 2H), 3.21-3.29 (m, 2H), 3.28-3.32 (m, 1H), 3.49-3.52 (m, 1H), 4.33-4.39 (m, 2H), 6.37 (s, 1H), 6.96-7.02 (m, 3H), 7.18 (d, J=7.20 Hz, 1H), 7.61 (t, J=7.60 Hz, 1H), 7.92-7.97 (m, 2H), 8.18 (s, 1H), 8.48 (s, 1H), 10.09 (s, 1H), 11.32 (br s, 1H). Mass spec m/z 679.4 [M+H]⁺.

### Step 3

### Intermediate 709: (R)-6-(4-((4-(4-((2-(1-methylpyrrolidin-2-yl)-1H-pyrrolo[3,2-c]pyridin-6-yl)carbamoyl)phenyl)piperazin-1-yl)methyl)piperidin-1-yl)picolinic acid

To a solution of tert-butyl (R)-6-(4-((4-(4-((2-(1-methylpyrrolidin-2-yl)-1H-pyrrolo[3,2-c]pyridin-6-yl)carbamoyl)phenyl)piperazin-1-yl)methyl)piperidin-1-yl)picolinate (Intermediate 708, 0.7 g, 1.03 mmol) in DCM was added trifluoroacetic acid (1.18 g, 10.31 mmol) and the resulting reaction mixture was stirred at room temperature for 16 h. After completion of the reaction, the reaction mixture was concentrated in vacuo to afford (R)-6-(4-((4-(4-((2-(1-methylpyrrolidin-2-yl)-1H-pyrrolo[3,2-c]pyridin-6-yl)carbamoyl)phenyl)piperazin-1-yl)methyl)piperidin-1-yl)picolinic acid (Intermediate 709, 0.520 g, 81%) as a brown solid which was used for next step without purification. Mass spec m/z 623.3 [M+H]⁺.

### Step 4

### Example 182: N—((S)-2,6-dioxopiperidin-3-yl)-6-(4-((4-(4-((2-((R)-1-methylpyrrolidin-2-yl)-1H-pyrrolo[3,2-c]pyridin-6-yl)carbamoyl)phenyl)piperazin-1-yl)methyl)piperidin-1-yl)picolinamide

To a solution of (R)-6-(4-((4-(4-((2-(1-methylpyrrolidin-2-yl)-1H-pyrrolo[3,2-c]pyridin-6-yl)carbamoyl)phenyl)piperazin-1-yl)methyl)piperidin-1-yl)picolinic acid (Intermediate 709, 0.3 g, 0.48 mmol) in dimethylformamide (2.0 mL) was added N,N-diisopropylethylamine (0.19 g, 1.45 mmol) at room temperature. After stirring for 5 min, HATU (0.29 g, 0.72 mmol) and (S)-3-aminopiperidine-2,6-dione hydrogen chloride (CAS No. 25181-50-4, 0.1 g, 0.6 mmol) were added and the resulting reaction mixture was stirred at room temperature for 16 h. After completion of the reaction, the reaction mixture was quenched with water (100 mL), the resultant precipitate was collected by filtration and dried in vacuo. The crude compound was purified purified by preparative HPLC (Method A) to afford N—((S)-2,6-dioxopiperidin-3-yl)-6-(4-((4-(4-((2-((R)-1-methylpyrrolidin-2-yl)-1H-pyrrolo[3,2-c]pyridin-6-yl)carbamoyl)phenyl)piperazin-1-yl)methyl)piperidin-1-yl)picolinamide (Example 182, 0.05 g, 14%) an off-white solid. ¹H NMR (400 MHz, DMSO-d₆) δ ppm 1.11-1.15 (m, 2H), 1.75-1.99 (m, 7H), 2.00-2.10-2.16 (m, 1H), 2.16 (s, 3H), 2.19-2.24 (m, 2H), 2.24-2.33 (m, 4H), 2.51-2.61 (m, 4H), 2.74-2.79 (m, 1H), 2.84-2.91 (m, 2H), 3.10-3.17 (m, 1H), 3.24-3.29 (m, 4H), 4.39-4.44 (m, 2H), 4.70-4.78 (m, 1H), 6.37 (s, 1H), 6.98 (d, J=8.80 Hz, 2H), 7.02-7.06 (m, 1H), 7.26 (d, J=7.20 Hz, 1H), 7.64-7.69 (m, 1H), 7.96 (d, J=9.20 Hz, 2H), 8.17 (s, 1H), 8.48 (s, 1H), 8.74 (d, J=8.40 Hz, 1H), 10.09 (s, 1H), 10.87 (s, 1H), 11.34 (br s, 1H). Mass spec m/z 733.1 [M+H]⁺.

### Example 183 was Synthesised Following Scheme 176

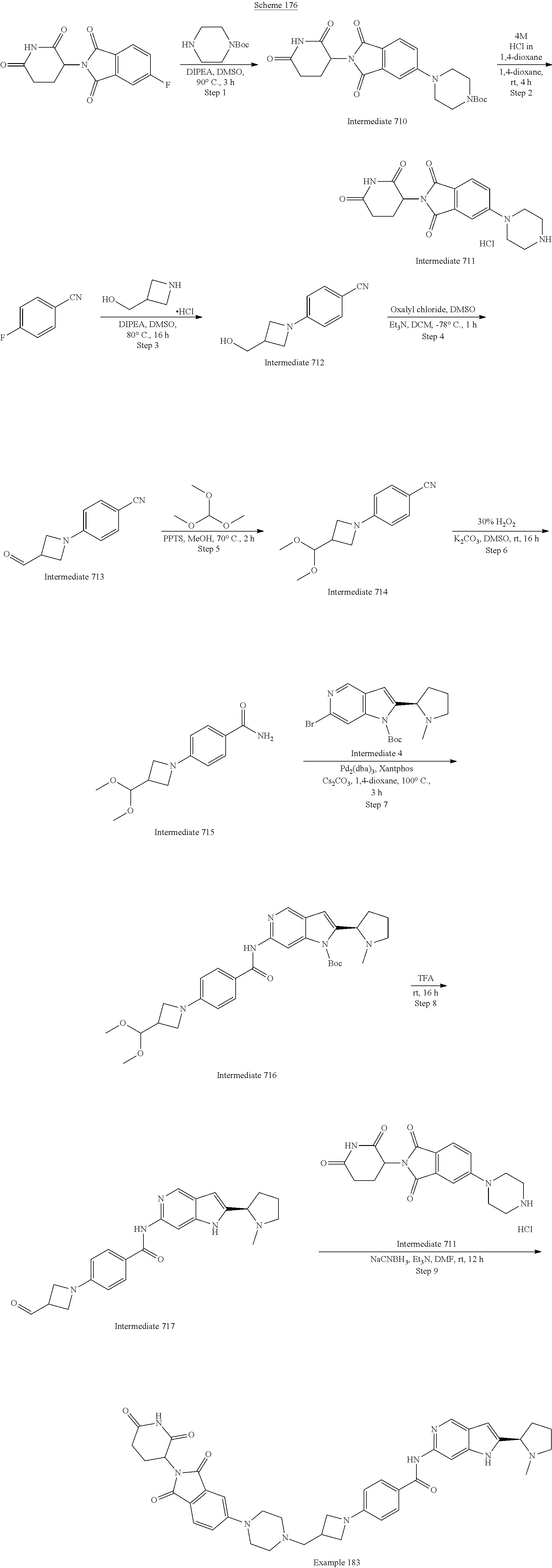

### Step 1

### Intermediate 710: tert-butyl 4-(2-(2,6-dioxopiperidin-3-yl)-1,3-dioxoisoindolin-5-yl)piperazine-1-carboxylate

To a solution of tert-butyl piperazine-1-carboxylate (CAS No. 57260-71-6, 2 g, 10.7 mmol) in dimethyl sulfoxide (20 mL) was added N,N-diisopropylethylamine (3.86 mL, 21.5 mmol) and the reaction mixture was stirred at room temperature for 10 min. 2-(2,6-Dioxopiperidin-3-yl)-5-fluoroisoindoline-1,3-dione (CAS No. 835616-61-0, 2.08 g, 7.52 mmol) was then added and the reaction mixture was stirred at 90° C. for 3 h. The reaction mixture was quenched with ice cold water (200 mL), the resultant precipitate was collected by filtration and dried in vacuo. The crude compound was purified by combi-flash chromatography, by eluting with 20% ethyl acetate in heptane, to afford tert-butyl 4-(2-(2,6-dioxopiperidin-3-yl)-1,3-dioxoisoindolin-5-yl)piperazine-1-carboxylate (Intermediate 710, 1.76 g, 37%) as a yellow solid. ¹H NMR (400 MHz, DMSO-d₆) δ ppm 0.97-1.07 (m, 2H), 1.36-1.42 (s, 9H), 1.97-2.03 (m, 1H), 2.33-2.36 (m, 1H), 2.52-2.67 (m, 3H), 2.82-2.91 (m, 1H), 3.43-3.50 (m, 4H), 5.04-5.08 (m, 1H), 7.22 (d, J=8.00 Hz, 1H), 7.32 (s, 1H), 7.67 (d, J=8.80 Hz, 1H), 11.05 (s, 1H). Mass spec m/z 443.6 [M+H]⁺.

### Step 2

### Intermediate 711: 2-(2,6-dioxopiperidin-3-yl)-5-(piperazin-1-yl)isoindoline-1,3-dione hydrochloride

To a cooled (0° C.) solution of tert-butyl 4-(2-(2,6-dioxopiperidin-3-yl)-1,3-dioxoisoindolin-5-yl)piperazine-1-carboxylate (CAS No. 928038-44-2, 1.5 g, 3.4 mmol) in dichloromethane (10 mL) was added 4M HCl in 1,4-dioxane (3.0 mL) and the reaction mixture was stirred at room temperature for 4 h. The reaction mixture was concentrated in vacuo. The obtained residue was triturated with diethyl ether (20 mL) and dried in vacuo to afford 2-(2,6-dioxopiperidin-3-yl)-5-(piperazin-1-yl)isoindoline-1,3-dione hydrochloride (Intermediate 711, 1.0 g) as an off-white solid. Mass spec m/z 343.3 [M+H]⁺.

### Step 3

### Intermediate 712: 4-(3-(hydroxymethyl)azetidin-1-yl)benzonitrile

To a solution of azetidin-3-ylmethanol hydrochloride (Intermediate 711, 9.49 g, 109 mmol) in dimethyl sulfoxide (100 mL) was added N,N-diisopropylethylamine (2.0 g, 199 mmol) and the reaction mixture was stirred at room temperature for 10 min. 4-Fluorobenzonitrile (CAS No. 1194-02-1, 12.0 g, 99.1 mmol) was then added and the reaction mixture was stirred at 80° C. for 16 h. The reaction mixture was quenched with ice cold water (200 mL), the resultant precipitate was collected by filtration and dried in vacuo. The crude material was purified by combi-flash chromatography, by eluting with 80% ethyl acetate in heptane, to afford 4-(3-(hydroxymethyl)azetidin-1-yl)benzonitrile (Intermediate 712, 7.0 g, 38%) as a white solid. ¹H NMR (400 MHz, DMSO-d₆) δ ppm 2.78-2.84 (m, 1H), 3.54-3.59 (m, 2H), 3.64-3.67 (m, 2H), 3.91-3.95 (m, 2H), 4.78-4.82 (m, 1H), 6.42 (d, J=8.80 Hz, 2H), 7.51 (d, J=8.40 Hz, 2H). Mass spec m/z 187.0 [M−H]⁻.

### Step 4

### Intermediate 713: 4-(3-formylazetidin-1-yl)benzonitrile

To a cooled (−78° C.) solution of oxalyl chloride (9.3 mL, 106 mmol) in dichloromethane (50 mL) was added dimethyl sulfoxide (15.2 mL, 213 mmol) followed by 4-(3-(hydroxymethyl)azetidin-1-yl)benzonitrile (Intermediate 712, 5.0 g, 26.6 mmol) and triethylamine (37.2 mL, 266 mmol) and the reaction mixture was stirred at −78° C. for 1 h. The reaction mixture was quenched with ice cold water (100 mL) and extracted with ethyl acetate (2×100 mL). The combined organic layers were dried over Na₂SO₄, filtered and concentrated in vacuo. The crude material was purified by combi-flash chromatography, by eluting with 80% ethyl acetate in heptane, to afford 4-(3-formylazetidin-1-yl)benzonitrile (Intermediate 713, 3.0 g, 61%) as an off-white solid. ¹H NMR (400 MHz, DMSO-d₆) δ ppm 3.25-3.35 (m, 1H), 3.64-3.73 (m, 2H), 3.75-3.91 (m, 2H), 6.42 (d, J=8.80 Hz, 2H), 7.51 (d, J=8.40 Hz, 2H), 9.8 (s, 1H).

### Step 5

### Intermediate 714: 4-(3-(dimethoxymethyl)azetidin-1-yl)benzonitrile

To a solution of 4-(3-(hydroxymethyl)azetidin-1-yl)benzonitrile (Intermediate 713, 2.5 g, 13 mmol) in methanol (25 mL) was added trimethyl orthoformate (2.9 g, 27 mmol) followed by pyridinium p-toluenesulfonate (0.47 g, 2.7 mmol) and the reaction mixture was stirred at 70° C. for 2 h. The reaction mixture was quenched with ice cold water (100 mL) and extracted with ethyl acetate (2×100 mL). The combined organic layers were dried over Na₂SO₄, filtered and concentrated in vacuo to afford 4-(3-(dimethoxymethyl)azetidin-1-yl)benzonitrile (Intermediate 714, 1.4 g, 45%) as an off-white solid which was used for next step without further purification. Mass spec m/z 233.0 [M+H]⁺.

### Step 6

### Intermediate 715: 4-(3-(dimethoxymethyl)azetidin-1-yl)benzamide

To a solution of 4-(3-(dimethoxymethyl)azetidin-1-yl)benzonitrile (Intermediate 714, 1.0 g, 4.30 mmol) in dimethyl sulfoxide (10 mL) was added potassium carbonate (1.48 g, 10.8 mmol) followed by 30% H₂O₂ (3.36 mL, 43.05 mmol) and the reaction mixture was stirred at room temperature for 16 h. The reaction mixture was quenched with ice cold water (100 mL), the resultant precipitate was collected by filtration and dried in vacuo to afford 4-(3-(dimethoxymethyl)azetidin-1-yl)benzamide (Intermediate 715, 0.850 g, 79%) as an off-white solid. ¹H NMR (400 MHz, DMSO-d₆) δ ppm 2.98-3.02 (m, 1H), 3.29 (s, 6H), 3.65-3.69 (m, 2H), 3.89-3.93 (m, 2H), 4.61-4.63 (m, 1H), 6.38 (d, J=8.80 Hz, 2H), 6.96 (br s, 1H), 7.64 (bs, 1H), 7.71 (d, J=8.80 Hz, 2H). Mass spec m/z 251.1 [M+H]⁺.

### Step 7

### Intermediate 716: tert-butyl (R)-6-(4-(3-(dimethoxymethyl)azetidin-1-yl)benzamido)-2-(1-methylpyrrolidin-2-yl)-1H-pyrrolo[3,2-c]pyridine-1-carboxylate

To a solution of 4-(3-(dimethoxymethyl)azetidin-1-yl)benzamide (Intermediate 715, 0.4 g, 1.59 mmol) in 1,4-dioxane (4.0 mL) was added tert-butyl (R)-6-bromo-2-(1-methylpyrrolidin-2-yl)-1H-pyrrolo[3,2-c]pyridine-1-carboxylate (Intermediate 4, 0.66 g, 1.75 mmol) followed by cesium carbonate (0.92 g, 4.79 mmol). The reaction mixture was purged with argon for 15 min, then Pd₂(dba)₃ (0.20 g 0.23 mmol) and Xantphos (0.23 g, 0.93 mmol) were added. The reaction mixture was further purged with argon for 10 min and heated at 100° C. for 3 h. The reaction mixture was concentrated in vacuo and the crude material was purified by combi-flash chromatography, by eluting with 10% methanol in DCM, to afford tert-butyl (R)-6-(4-(3-(dimethoxymethyl)azetidin-1-yl)benzamido)-2-(1-methylpyrrolidin-2-yl)-1H-pyrrolo[3,2-c]pyridine-1-carboxylate (Intermediate 716, 0.8 g, 44%) as an off-white solid. ¹H NMR (400 MHz, DMSO-d₆) δ ppm 1.55-1.85 (m, 12H), 2.28-2.41 (m, 5H), 2.98-3.06 (m, 1H), 3.09-3.13 (m, 1H), 3.29 (s, 6H), 3.69-3.73 (m, 2H), 3.89-4.03 (m, 3H), 4.61-4.63 (m, 1H), 6.43 (d, J=8.80 Hz, 2H), 6.72 (s, 1H), 7.93 (d, J=8.80 Hz, 2H), 8.55 (s, 1H), 8.91 (s, 1H), 10.26 (s, 1H). Mass spec m/z 550.3 [M+H]⁺.

### Step 8

### Intermediate 717: (R)-4-(3-formylazetidin-1-yl)-N-(2-(1-methylpyrrolidin-2-yl)-1H-pyrrolo[3,2-c]pyridin-6-yl)benzamide trifluoroacetate

A solution of tert-butyl (R)-6-(4-(3-(dimethoxymethyl)azetidin-1-yl)benzamido)-2-(1-methylpyrrolidin-2-yl)-1H-pyrrolo[3,2-c]pyridine-1-carboxylate (Intermediate 716, 0.3 g, 0.54 mmol) in trifluroacetic acid (3.0 ml) was stirred at room temperature for 16 h. The reaction mixture was concentrated in vacuo to afford (R)-4-(3-formylazetidin-1-yl)-N-(2-(1-methylpyrrolidin-2-yl)-1H-pyrrolo[3,2-c]pyridin-6-yl)benzamide trifluoroacetate (Intermediate 717, 0.2 g, 73%) as an off-white solid which was used for next step without purification. Mass spec m/z 402.0 [M−H]⁻.

### Step 9

### Example 183: 4-(3-((4-(2-(2,6-dioxopiperidin-3-yl)-1,3-dioxoisoindolin-5-yl)piperazin-1-yl)methyl)azetidin-1-yl)-N-(2-((R)-1-methylpyrrolidin-2-yl)-1H-pyrrolo[3,2-c]pyridin-6-yl)benzamide

To a solution of (R)-4-(3-formylazetidin-1-yl)-N-(2-(1-methylpyrrolidin-2-yl)-1H-pyrrolo[3,2-c]pyridin-6-yl)benzamide trifluoroacetate (Intermediate 717, 0.1 g, 0.19 mmol) in dimethylformamide (0.5 mL) was added triethylamine (0.140 mL, 0.99 mmol) followed by 2-(2,6-dioxopiperidin-3-yl)-5-(piperazin-1-yl)isoindoline-1,3-dione hydrochloride (Intermediate 711, 0.075 g, 0.19 mmol) and the reaction mixture was stirred at room temperature for 3 h. Sodium cyanoborohydride (0.039 g 0.59 mmol) was then added and reaction mixture was stirred at room temperature for 16 h. The reaction mixture was quenched with ice cold water (100 mL), the resultant precipitate was collected by filtration and dried in vacuo. The crude material was purified by preparative HPLC (Method A) to afford 4-(3-((4-(2-(2,6-dioxopiperidin-3-yl)-1,3-dioxoisoindolin-5-yl)piperazin-1-yl)methyl)azetidin-1-yl)-N-(2-((R)-1-methylpyrrolidin-2-yl)-1H-pyrrolo[3,2-c]pyridin-6-yl)benzamide (Example 183, 0.030 g, 21%) as an off-white solid. ¹—H NMR (400 MHz, DMSO-d₆) δ ppm 1.76-191 (m, 3H), 1.98-2.06 (m, 1H), 2.13-2.97 (m, 4H), 2.22-2.29 (m, 1H), 2.25-2.59 (m, 5H), 2.66-2.69 (m, 2H), 2.83-2.92 (m, 1H), 2.97-3.04 (m, 1H), 3.12-3.17 (m, 1H), 3.27-3.29 (m, 2H), 3.46-3.48 (m, 4H), 3.58-3.62 (m, 2H), 4.04-4.08 (m, 2H), 5.05-5.09 (m, 1H), 6.37 (s, 1H), 6.43 (d, J=8.80 Hz, 2H), 7.27 (d, J=8.80 Hz, 1H), 7.35 (s, 1H), 7.78 (d, J=8.40 Hz, 1H), 7.93 (d, J=8.80 Hz, 2H), 8.16 (s, 1H), 8.47 (s, 1H), 10.00 (s, 1H), 11.07 (s, 1H), 11.32 (s, 1H). Mass spec m/z 730.3 [M+H]⁺.

### Example 184 was Synthesised Following Scheme 177

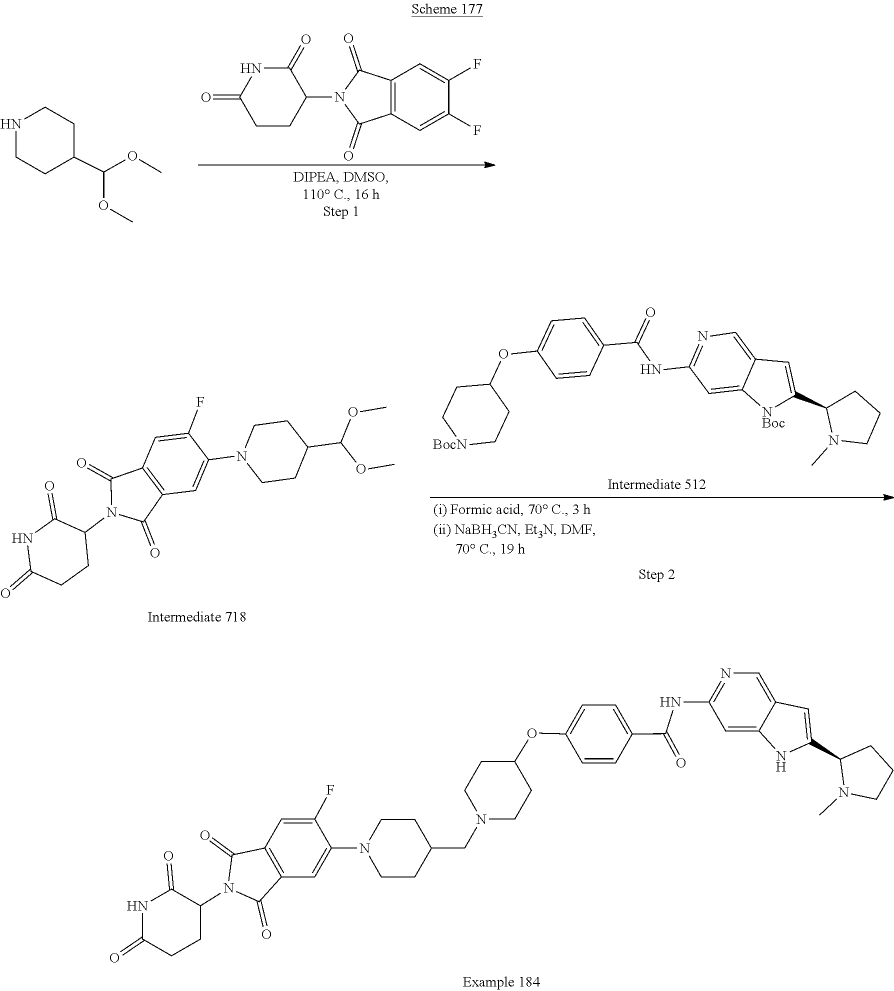 [SMILES: COC(OC)C1CCNCC1 ; O=C1CCC(N2C(=O)c3cc(F)c(F)cc3C2=O)C(=O)N1 ; COC(OC)C1CCN(c2cc3c(cc2F)C(=O)N(C2CCC(=O)NC2=O)C3=O)CC1 ; CN1CCC[C@@H]1c1cc2cnc(NC(=O)c3ccc(OC4CCN(C(=O)OC(C)(C)C)CC4)cc3)cc2n1C(=O)OC(C)(C)C ; CN1CCC[C@@H]1c1cc2cnc(NC(=O)c3ccc(OC4CCN(CC5CCN(c6cc7c(cc6F)C(=O)N(C6CCC(=O)NC6=O)C7=O)CC5)CC4)cc3)cc2[nH]1]

### Step 1

### Intermediate 718: 5-(4-(dimethoxymethyl)piperidin-1-yl)-2-(2,6-dioxopiperidin-3-yl)-6-fluoroisoindoline-1,3-dione

To a solution of 4-(dimethoxymethyl)piperidine (CAS No. 188646-83-5, 1.08 g, 6.46 mmol) in dimethyl sulfoxide (15 mL) was added N,N-diisopropylethylamine (3.38 mL, 19.40 mmol) and 2-(2,6-dioxopiperidin-3-yl)-5,6-difluoroisoindoline-1,3-dione (CAS No. 1496997-41-1, 2.0 g, 6.46 mmol). The reaction mixture was heated at 110° C. for 16 h. After completion, the reaction mixture was concentrated in vacuo. The crude material was purified by combi-flash chromatography, by eluting with 80% ethyl acetate in heptane, to afford 5-(4-(dimethoxymethyl)piperidin-1-yl)-2-(2,6-dioxopiperidin-3-yl)-6-fluoroisoindoline-1,3-dione (Intermediate 718, 1.8 g, 55%) as an off-white solid. ¹H NMR (400 MHz, DMSO-d₆) δ ppm 1.37-1.49 (m, 3H), 1.71-1.81 (m, 3H), 1.91-2.05 (m, 1H), 2.52-2.56 (m, 3H), 2.85-2.95 (m, 3H), 3.26 (s, 6H), 4.10-4.20 (m, 1H), 5.07-5.15 (m, 1H), 7.44 (d, J=7.38 Hz, 1H), 7.70 (d, J=7.58 Hz, 1H), 11.10 (br s, 1H). Mass spec m/z 434.1 [M+H]⁺.

### Step 2

### Example 184: 4-((1-((1-(2-(2,6-dioxopiperidin-3-yl)-6-fluoro-1,3-dioxoisoindolin-5-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide

A solution of 5-(4-(dimethoxymethyl)piperidin-1-yl)-2-(2,6-dioxopiperidin-3-yl)-6-fluoroisoindoline-1,3-dione (Intermediate 718, 0.50 g, 1.15 mmol) in formic acid (1 mL) was heated at 70° C. for 3 h. The reaction mixture was concentrated in vacuo. A solution of tert-butyl (R)-6-(4-((1-(tert-butoxycarbonyl)piperidin-4-yl)oxy)benzamido)-2-(1-methylpyrrolidin-2-yl)-1H-pyrrolo[3,2-c]pyridine-1-carboxylate (Intermediate 512, 0.71 g, 1.15 mmol) in dimethylformamide (1 mL) and triethylamine (0.80 mL, 5.76 mmol) were then added and the reaction mixture was heated at 70° C. for 3 h. Sodium cyanoborohydride (0.30 g, 4.61 mmol) was then added and heating continued at 70° C. for 16 h. After completion, the reaction mixture was diluted with ice cold water (30 mL), stirred for 10 min, the resultant precipitate was collected by filtration and dried in vacuo. The crude material was purified by preparative HPLC (Method B) to afford 4-((1-((1-(2-(2,6-dioxopiperidin-3-yl)-6-fluoro-1,3-dioxoisoindolin-5-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide (Example 184, 0.16 g, 17%) as a yellow solid. ¹H NMR (400 MHz, DMSO-d₆) δ ppm 1.25-1.27 (m, 2H), 1.63-1.69 (m, 2H), 1.73-2.04 (m, 10H), 2.13-2.15 (m, 1H), 2.16 (s, 3H), 2.20-2.29 (m, 5H), 2.53-2.58 (m, 1H), 2.69-2.73 (m, 2H), 2.85-2.93 (m, 3H), 3.11-3.17 (m, 1H), 3.27-3.29 (m, 1H), 3.58-3.65 (m, 2H), 4.49-4.55 (m, 1H), 5.07-5.12 (m, 1H), 6.38 (s, 1H), 7.03 (d, J=9.01 Hz, 2H), 7.44 (d, J=7.38 Hz, 1H), 7.44 (d, J=7.38 Hz, 1H), 7.67-7.73 (m, 2H), 8.17 (s, 1H), 8.49 (s, 1H), 10.27 (br s, 1H), 11.10 (br s, 1H), 11.34 (br s, 1H). Mass spec m/z 791.1 [M+H]⁺.

### Example 185 was Synthesised Following Scheme 178

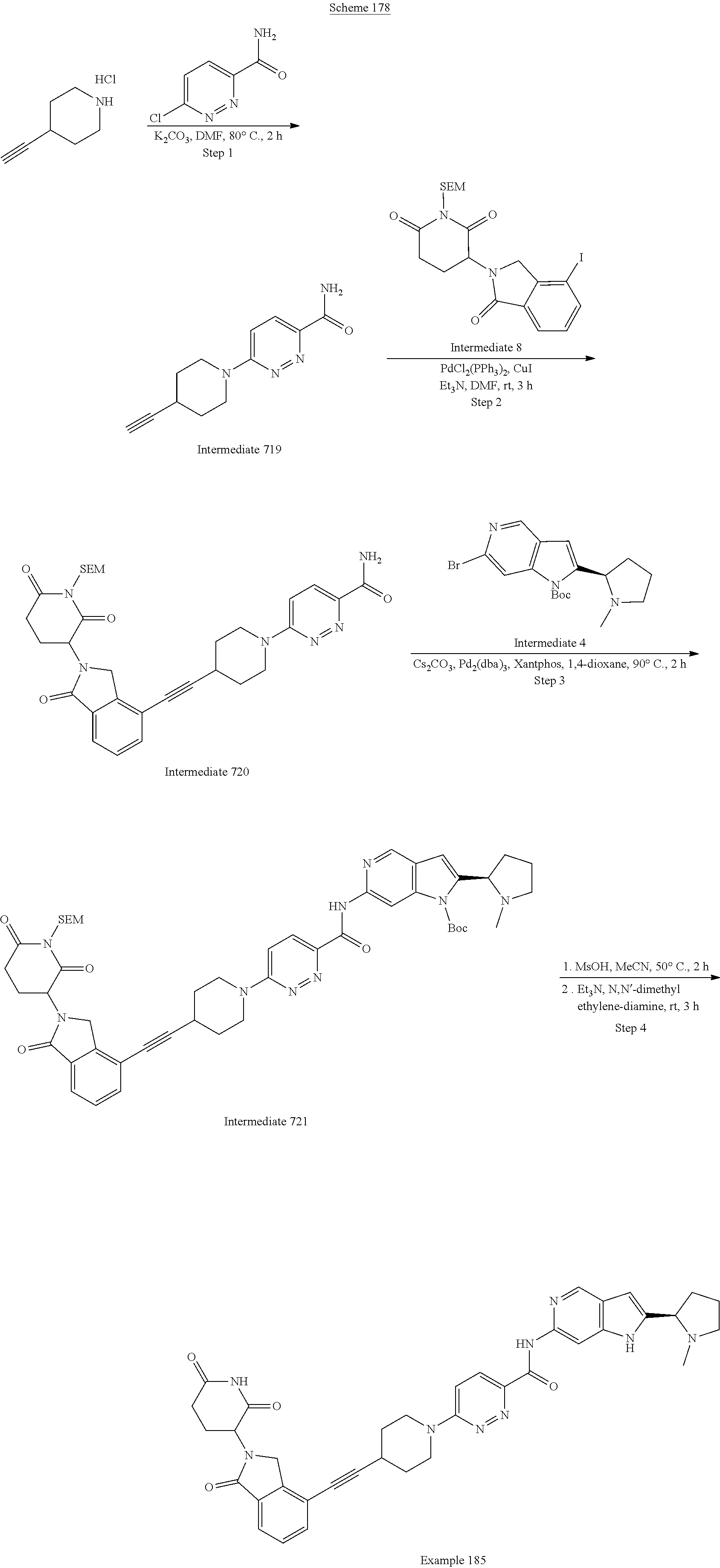

### Step 1

### Intermediate 719: 6-(4-ethynylpiperidin-1-yl)pyridazine-3-carboxamide

To a solution of 4-ethynylpiperidine hydrochloride (CAS No: 550378-30-8, 0.92 g, 6.34 mmol) and 6-chloropyridazine-3-carboxamide (CAS No: 66346-83-6, 1 g, 6.34 mmol) in dimethylformamide (10 mL) was added cesium carbonate (3.0 g, 9.20 mmol) and the reaction was heated to 80° C. for 2 h. After completion, the reaction mixture was quenched with cold water (50 mL), resulting in a solid precipitate, which was collected by filtration, and dried in vacuo to afford 6-(4-ethynylpiperidin-1-yl)pyridazine-3-carboxamide (Intermediate 719, 3.5 g) as an off-white solid, which was used for next step without further purification. ¹H NMR (400 MHz, DMSO-d₆) δ ppm 1.54-1.56 (m, 2H), 1.82-1.88 (m, 2H), 2.72-2.77 (m, 1H), 2.98-3.10 (m, 1H), 3.39-3.47 (m, 2H), 4.06-4.09 (m, 2H), 7.35 (d, J=8.54 Hz, 1H), 7.53 (br s, 1H), 7.82 (d, J=9.54 Hz, 1H), 8.14 (br s, 1H). Mass spec m/z 231.4 [M+H]⁺.

### Step 2

### Intermediate 720: 6-(4-((2-(2,6-dioxo-1-((2-(trimethylsilyl)ethoxy)methyl)piperidin-3-yl)-1-oxoisoindolin-4-yl)ethynyl)piperidin-1-yl)pyridazine-3-carboxamide

To a solution of 3-(4-iodo-1-oxoisoindolin-2-yl)-1-((2-(trimethylsilyl)ethoxy)methyl)piperidine-2,6-dione (Intermediate 8, 2.17 g, 4.34 mmol) and 6-(4-ethynylpiperidin-1-yl)pyridazine-3-carboxamide (Intermediate 719, 1.0 g, 4.34 mmol) in dimethylformamide (10 mL) was add triethylamine (21.9 mL, 156.34 mmol). The reaction mixture was purged with argon for 15 min, then PdCl₂(PPh₃)₂ (0.30 g, 0.43 mmol) and copper(I) iodide (0.08 g, 0.43 mmol) were added. The reaction mixture was further purged with argon for 10 min and then stirred at room temperature for 3 h. After completion, the reaction mixture was concentrated in vacuo, diluted with water (300 mL) and extracted with ethyl acetate (3×200 mL). The combined organic layers were dried over anhydrous Na₂SO₄, filtered and concentrated in vacuo. The crude material was purified by combi-flash chromatography, by eluting with 90% ethyl acetate in heptane, to afford 6-(4-((2-(2,6-dioxo-1-((2-(trimethylsilyl)ethoxy)methyl)piperidin-3-yl)-1-oxoisoindolin-4-yl)ethynyl)piperidin-1-yl)pyridazine-3-carboxamide (Intermediate 720, 1.5 g, 57%) as a yellow liquid. ¹H NMR (400 MHz, DMSO-d₆) δ ppm −0.03 (s, 9H), 0.77-0.86 (m, 2H), 1.64-1.73 (m, 2H), 1.90-2.08 (m, 3H), 2.41-2.50 (m, 2H), 2.75-2.87 (m, 1H), 3.02-3.09 (m, 2H), 3.48-3.57 (m, 4H), 4.13-4.18 (m, 2H), 4.25-4.30 (m, 1H), 4.44-4.50 (m, 1H), 5.00-5.08 (m, 2H), 7.37 (d, J=9.54 Hz, 1H), 7.51-7.58 (m, 2H), 7.67 (d, J=8.57 Hz, 1H), 7.73 (d, J=8.57 Hz, 1H), 7.83 (d, J=9.66 Hz, 1H), 8.12 (br s, 1H). Mass spec m/z 603.0 [M+H]⁺.

### Step 3

### Intermediate 721: tert-butyl 6-(6-(4-((2-(2,6-dioxo-1-((2-(trimethylsilyl)ethoxy)methyl)piperidin-3-yl)-1-oxoisoindolin-4-yl)ethynyl)piperidin-1-yl)pyridazine-3-carboxamido)-2-((R)-1-methylpyrrolidin-2-yl)-1H-pyrrolo[3,2-c]pyridine-1-carboxylate

To a solution of 6-(4-((2-(2,6-dioxo-1-((2-(trimethylsilyl)ethoxy)methyl)piperidin-3-yl)-1-oxoisoindolin-4-yl)ethynyl)piperidin-1-yl)pyridazine-3-carboxamide (Intermediate 720, 0.63 g, 1.052 mmol) and tert-butyl (R)-6-bromo-2-(1-methylpyrrolidin-2-yl)-1H-pyrrolo[3,2-c]pyridine-1-carboxylate (Intermediate 4, 0.50 g, 1.3 mmol) in 1,4-dioxane (15 mL) was added cesium carbonate (1.28 g, 3.94 mmol). The reaction mixture was purged with argon for 15 min, then Xantphos (0.22 g, 0.39 mmol) and Pd₂(dba)₃ (0.18 g, 0.19 mmol) were added. The reaction mixture was further purged with argon for 10 min and then heated at 90° C. for 2 h. After completion, the reaction mixture was filtered through a celite bed, the filter pad was washed with ethyl acetate (100 mL) and the filtrate concentrated in vacuo. The crude material was purified by combi-flash chromatography, by eluting with 90% ethyl acetate in heptane, to afford tert-butyl 6-(6-(4-((2-(2,6-dioxo-1-((2-(trimethylsilyl)ethoxy)methyl)piperidin-3-yl)-1-oxoisoindolin-4-yl)ethynyl)piperidin-1-yl)pyridazine-3-carboxamido)-2-((R)-1-methylpyrrolidin-2-yl)-1H-pyrrolo[3,2-c]pyridine-1-carboxylate (Intermediate 721, 0.27 g, 23%) as a yellow solid. ¹H NMR (401 MHz, DMSO-d₆) δ ppm −0.05 (s, 9H), 0.77-0.84 (m, 2H), 1.66-1.69 (m, 1H), 1.71 (s, 9H), 1.72-1.79 (m, 4H), 1.99-2.10 (m, 3H), 2.30-2.34 (m, 2H), 2.35 (s, 3H), 3.04-3.24 (m, 4H), 3.47-3.66 (m, 4H), 4.10-4.40 (m, 5H), 4.42-4.54 (m, 1H), 4.98-5.11 (m, 2H), 5.22-5.31 (m, 1H), 6.46 (s, 1H), 7.29-7.45 (m, 1H), 7.48-7.57 (m, 2H), 7.58-7.76 (m, 1H), 7.79-8.03 (m, 1H), 8.12 (s, 1H), 8.59 (s, 1H), 10.28 (br s, 1H). Mass spec m/z 901.6 [M]⁺.

### Step 4

### Example 185: 6-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)pyridazine-3-carboxamide

To a stirred solution of 6-(6-(4-((2-(2,6-dioxo-1-((2-(trimethylsilyl)ethoxy)methyl)piperidin-3-yl)-1-oxoisoindolin-4-yl)ethynyl)piperidin-1-yl)pyridazine-3-carboxamido)-2-((R)-1-methylpyrrolidin-2-yl)-1H-pyrrolo[3,2-c]pyridine-1-carboxylate (Intermediate 721, 0.27 g, 0.29 mmol) in acetonitrile (10 mL) was added methanesulfonic acid (0.1 mL, 2.99 mmol) and the reaction heated at 50° C. for 2 h. The reaction mixture was cooled to room temperature then N,N-dimethylethylenediamine (0.17 mL, 1.49 mmol) followed by triethylamine (0.83 mL, 5.98 mmol) were added and the reaction mixture was stirred at room temperature for 3 h. The reaction mixture was concentrated in vacuo, diluted with water (50 mL), resulting in a solid precipitate, which was collected by filtration, and dried in vacuo. The crude compound was purified by preparative HPLC (Method A) to afford 6-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)pyridazine-3-carboxamide (Example 185, 0.01 g, 8%) as an off-white solid. ¹H NMR (400 MHz, DMSO-d₆) δ ppm 1.71-1.92 (m, 5H), 2.00-2.06 (m, 3H), 2.13-2.15 (m, 1H), 2.16 (s, 3H), 2.23-2.30 (m, 1H), 2.31-2.34 (m, 1H), 2.55-2.62 (m, 2H), 2.86-2.96 (m, 1H), 3.09-3.16 (m, 2H), 3.59-3.64 (m, 2H), 4.17-4.21 (m, 2H), 4.31-4.36 (m, 1H), 4.44-4.49 (m, 1H), 5.10-5.14 (m, 1H), 6.40 (s, 1H), 7.46-7.50 (m, 1H), 7.51-7.54 (m, 1H), 7.67 (d, J=7.63 Hz, 1H), 7.71 (d, J=8.75 Hz, 1H), 8.00 (d, J=9.51 Hz, 1H), 8.23 (s, 1H), 8.49 (s, 1H), 10.11 (br s, 1H), 10.97 (br s, 1H), 11.42 (br s, 1H). Mass spec m/z 672.0 [M+H]⁺.

### Example 186 was Synthesised Following Scheme 179

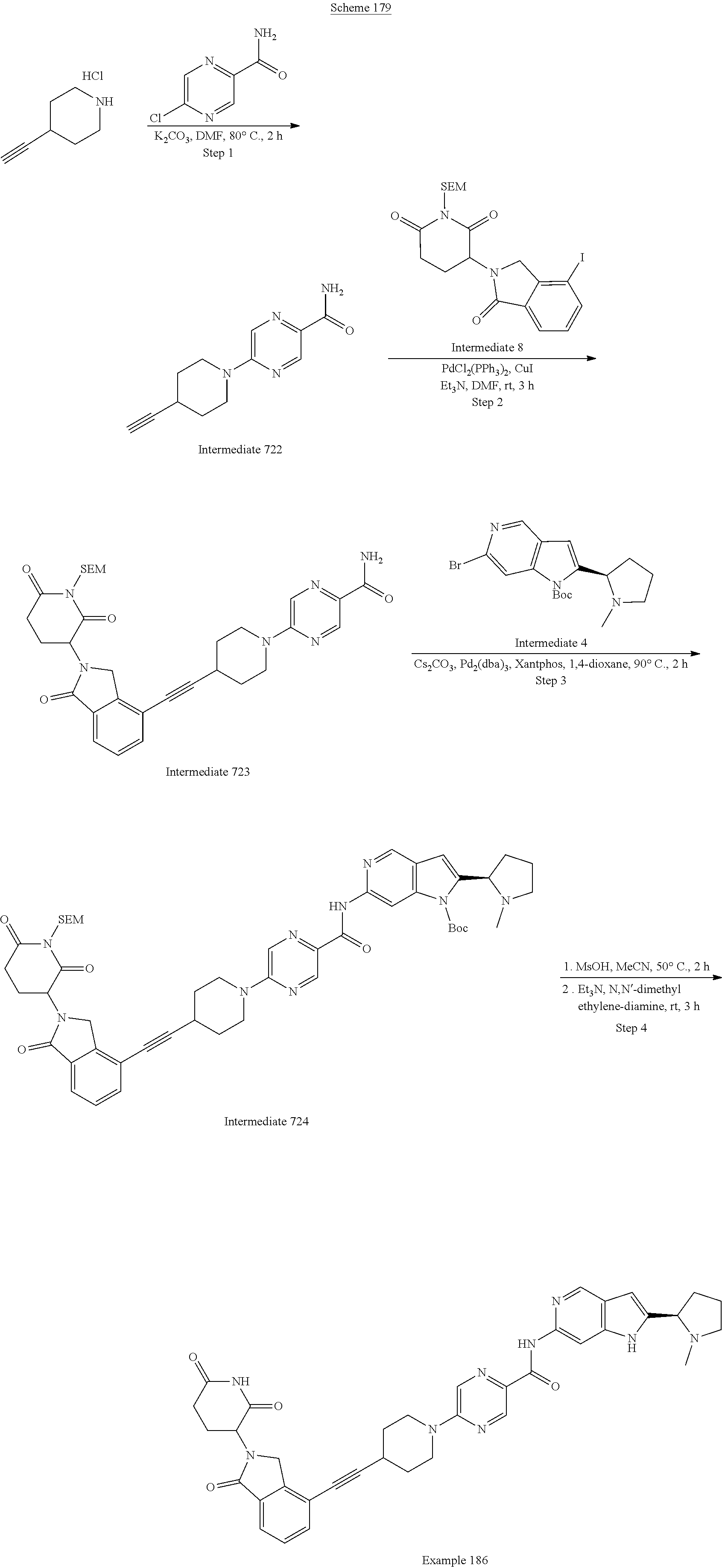

### Step 1

### Intermediate 722: 5-(4-ethynylpiperidin-1-yl)pyrazine-2-carboxamide

To a solution of 4-ethynylpiperidine hydrochloride (CAS No: 550378-30-8, 1.0 g, 6.35 mmol) in dimethylformamide (10 mL) was added 5-chloropyrazine-2-carboxamide (CAS No: 21279-64-1, 1 g, 6.35 mmol) and cesium carbonate (8.8 g, 25.38 mmol) and the reaction mixture was stirred at 80° C. for 2 h. After completion, the reaction mixture was quenched with ice cold water (200 mL), the resultant precipitate was collected by filtration and dried in vacuo to afford 5-(4-ethynylpiperidin-1-yl)pyrazine-2-carboxamide (Intermediate 722, 1.6 g) as a white solid, which was used in the next step without further purification. ¹H NMR (400 MHz, DMSO-d₆) δ ppm 1.50-1.58 (m, 2H), 1.85-1.88 (m, 2H), 2.73-2.75 (m, 1H), 3.00 (s, 1H), 3.40-3.46 (m, 2H), 4.02-4.06 (m, 2H), 7.34 (s, 1H), 7.70 (s, 1H), 8.27 (s, 1H), 8.60 (s, 1H). Mass spec m/z 231.4 [M+H]⁺.

### Step 2

### Intermediate 723: 5-(4-((2-(2,6-dioxo-1-((2-(trimethylsilyl)ethoxy)methyl)piperidin-3-yl)-1-oxoisoindolin-4-yl)ethynyl)piperidin-1-yl)pyrazine-2-carboxamide

To a solution of 3-(4-iodo-1-oxoisoindolin-2-yl)-1-((2-(trimethylsilyl)ethoxy)methyl)piperidine-2,6-dione (Intermediate 8, 2.2 g, 4.3 mmol) and 5-(4-ethynylpiperidin-1-yl)pyrazine-2-carboxamide (Intermediate 722, 1.6 g, 4.3 mmol) in dimethylformamide (10 mL) was added triethylamine (21.9 mL, 156.3 mmol). The reaction mixture was purged with argon for 15 min, then PdCl₂(PPh₃)₂ (0.31 g, 0.43 mmol) and copper(I) iodide (0.084 g, 0.43 mmol) were added. The reaction mixture was further purged with argon for 10 min and stirred at room temperature for 3 h. After completion, the reaction mixture was concentrated in vacuo, diluted with water (300 mL) and extracted with ethyl acetate (3×200 mL). The combined organic layers were dried over anhydrous Na₂SO₄ and concentrated in vacuo to obtain crude material. The crude material was purified by combi-flash chromatography, by eluting with 90% ethyl acetate in heptane, to afford 5-(4-((2-(2,6-dioxo-1-((2-(trimethylsilyl)ethoxy)methyl)piperidin-3-yl)-1-oxoisoindolin-4-yl)ethynyl)piperidin-1-yl)pyrazine-2-carboxamide (Intermediate 723, 1.6 g, 61%) as a yellow liquid. ¹H NMR (400 MHz, DMSO-d₆) δ ppm −0.048 (s, 9H), 0.80-0.84 (m, 2H), 1.65-1.70 (m, 2H), 1.94-2.08 (m, 3H), 2.37-2.45 (m, 1H), 2.77-2.81 (m, 1H), 3.02-3.12 (m, 2H), 3.49-3.56 (m, 4H), 4.10-4.15 (m, 2H), 4.25-4.30 (m, 1H), 4.46-4.50 (m, 1H), 5.00-5.08 (m, 2H), 5.24-5.28 (m, 1H), 7.35 (s, 1H), 7.35 (t, J=7.4 Hz, 1H), 7.66-7.74 (m, 3H), 8.29 (s, 1H), 8.61 (s, 1H). Mass spec m/z 603.3 [M+H]⁺.

### Step 3

### Intermediate 724: tert-butyl 6-(5-(4-((2-(2,6-dioxo-1-((2-(trimethylsilyl)ethoxy)methyl)piperidin-3-yl)-1-oxoisoindolin-4-yl)ethynyl)piperidin-1-yl)pyrazine-2-carboxamido)-2-((R)-1-methylpyrrolidin-2-yl)-1H-pyrrolo[3,2-c]pyridine-1-carboxylate

To a solution of 5-(4-((2-(2,6-dioxo-1-((2-(trimethylsilyl)ethoxy)methyl)piperidin-3-yl)-1-oxoisoindolin-4-yl)ethynyl)piperidin-1-yl)pyrazine-2-carboxamide (Intermediate 723, 0.63 g, 1.05 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 0.5 g, 1.31 mmol) and cesium carbonate (1.28 g, 3.94 mmol). The reaction mixture was purged with argon for 15 min, then Xanthphos (0.23 g, 0.39 mmol) and Pd₂(dba)₃ (0.18 g, 0.19 mmol) were added. The reaction was purged with argon for a further 10 min and heated at 90° C. for 2 h. After completion, the reaction mixture was filtered through a celite bed, the filter pad was washed with ethyl acetate (100 mL), and the filtrate was concentrated in vacuo. The crude material was purified by combi-flash chromatography, by eluting with 90% ethyl acetate in heptane, to afford tert-butyl 6-(5-(4-((2-(2,6-dioxo-1-((2-(trimethylsilyl)ethoxy)methyl)piperidin-3-yl)-1-oxoisoindolin-4-yl)ethynyl)piperidin-1-yl)pyrazine-2-carboxamido)-2-((R)-1-methylpyrrolidin-2-yl)-1H-pyrrolo[3,2-c]pyridine-1-carboxylate (Intermediate 724, 0.27 g, 23%) as a yellow solid. ¹H NMR (400 MHz, DMSO-d₆) δ ppm −0.05 (s, 9H), 0.78-0.86 (m, 2H), 1.60-1.79 (m, 14H), 2.03-2.10 (m, 3H), 2.33-2.35 (m, 5H), 2.78-2.82 (m, 1H), 3.05-3.16 (m, 3H), 3.50-3.63 (m, 5H), 3.80-3.86 (m, 1H), 4.16-4.20 (m, 2H), 4.26-4.32 (m, 1H), 4.49-4.52 (m, 1H), 5.00-5.10 (m, 2H), 5.24-5.30 (m, 1H), 6.74 (s, 1H), 7.53-7.57 (m, 1H), 7.67-7.69 (m, 1H), 7.72-7.74 (m, 1H), 8.43 (s, 1H), 8.56 (s, 1H), 8.76 (s, 1H), 8.94 (s, 1H), 9.2 (s, 1H). Mass spec m/z 901.9 [M−H]⁻.

### Step 4

### Example 186: 5-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)pyrazine-2-carboxamide

To a solution of tert-butyl 6-(5-(4-((2-(2,6-dioxo-1-((2-(trimethylsilyl)ethoxy)methyl)piperidin-3-yl)-1-oxoisoindolin-4-yl)ethynyl)piperidin-1-yl)pyrazine-2-carboxamido)-2-((R)-1-methylpyrrolidin-2-yl)-1H-pyrrolo[3,2-c]pyridine-1-carboxylate (Intermediate 724, 0.27 g, 0.29 mmol) in acetonitrile (10 mL) was added methanesulfonic acid (0.19 mL, 2.99 mmol) and the reaction heated at 50° C. for 2 h. The reaction mixture was cooled to room temperature, then N,N′-dimethylethylenediamine (0.17 mL, 1.49 mmol) followed by triethylamine (0.83 mL, 5.98 mmol) were added and the reaction mixture was stirred at room temperature for 3 h. After completion, the reaction mixture was diluted with water (50 mL), resulting in a solid precipitate, which was collected by filtration and dried in vacuo. The crude material was purified by preparative HPLC (Method A) to afford 5-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)pyrazine-2-carboxamide (Example 186, 0.03 g, 15%) as a white solid. ¹H NMR (400 MHz, DMSO-d₆) δ ppm 1.58-1.91 (m, 6H), 2.95-2.02 (m, 4H), 2.12-2.17 (m, 4H), 2.21-2.28 (m, 1H), 2.58-2.61 (m, 2H), 2.76-2.92 (m, 1H), 3.08-3.15 (m, 2H), 3.58-3.63 (m, 2H), 4.14-4.18 (m, 2H), 4.29-4.36 (m, 1H), 4.45-4.49 (m, 1H), 5.10-5.15 (m, 1H), 6.39 (s, 1H), 7.53 (t, J=8.0 Hz, 1H), 7.67 (d, J=5.2 Hz, 1H), 7.72 (d, J=7.6 Hz, 1H), 8.43-8.46 (m, 2H), 8.77 (s, 1H), 9.77 (s, 1H), 10.99 (s, 1H), 10.42 (s, 1H). Mass spec m/z 672.9 [M+H]⁺.

### Example 187 was Synthesised Following Scheme 180

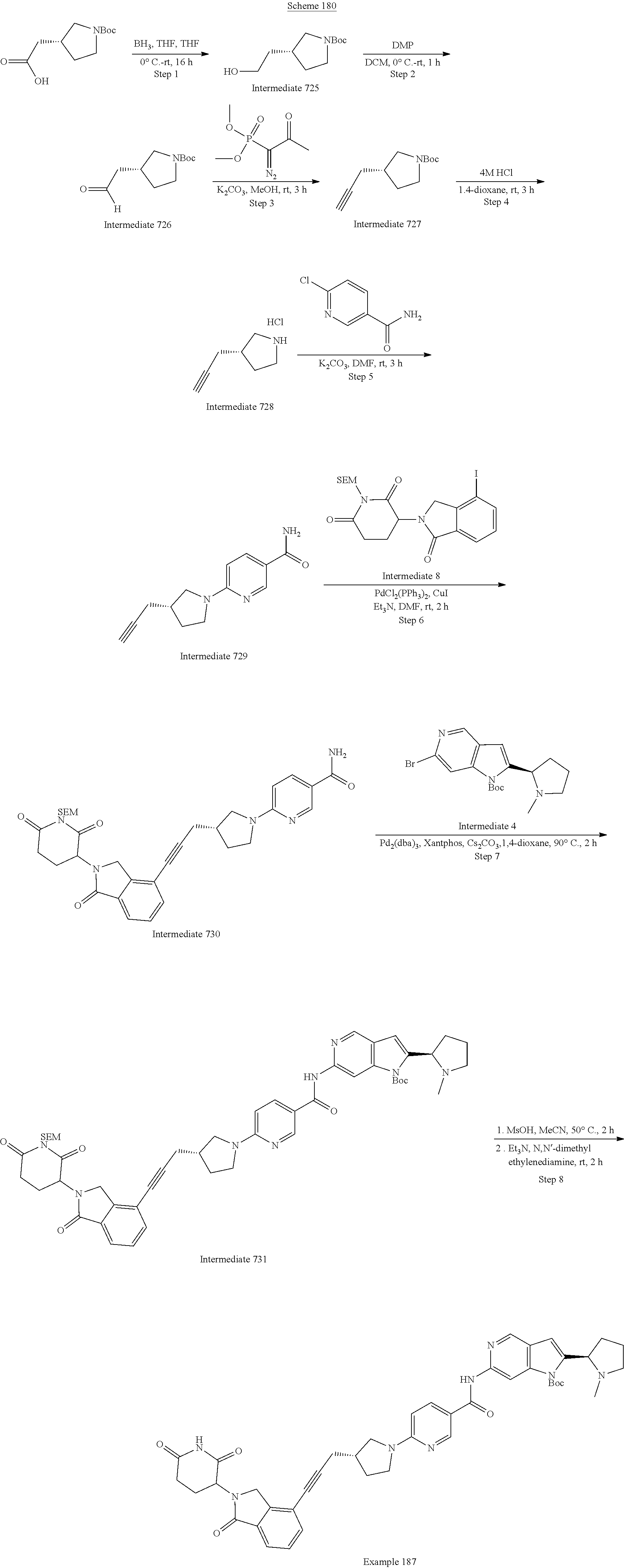

### Step 1

### Intermediate 725: tert-butyl (R)-3-(2-hydroxyethyl)pyrrolidine-1-carboxylate

To a cooled (0° C.) solution of (R)-2-(1-(tert-butoxycarbonyl)pyrrolidin-3-yl)acetic acid (CAS No: 204688-60-8, 5.0 g, 20.38 mmol) in tetrahydrofuran (50 mL) was added 1M solution of borane tetrahydrofuran complex in tetrahydrofuran (61.0 mL, 61.14 mmol) and reaction mixture was stirred at room temperature for 16 h. After completion, the reaction mixture was quenched with methanol (100 mL) at 0° C. and concentrated in vacuo. The crude material was purified by combi-flash chromatography, by eluting with 60% ethyl acetate in heptane, to afford tert-butyl (R)-3-(2-hydroxyethyl)pyrrolidine-1-carboxylate (Intermediate 725, 4.1 g, 87%) as a yellow liquid. ¹H NMR (400 MHz, DMSO-d₆) δ ppm 1.38 (s, 9H), 1.44-1.52 (m, 3H), 1.88-1.93 (m, 1H), 2.12-2.18 (m, 1H), 2.71-2.77 (m, 1H), 3.06-3.18 (m, 1H), 3.28-3.33 (m, 1H), 3.40-3.46 (m, 3H), 4.39-4.47 (m, 1H).

### Step 2

### Intermediate 726: tert-butyl (R)-3-(2-oxoethyl)pyrrolidine-1-carboxylate

To a cooled (0° C.) solution of tert-butyl (R)-3-(2-hydroxyethyl)pyrrolidine-1-carboxylate (Intermediate 725, 4.10 g, 18.0 mmol) in dichloromethane (50 mL) was added Dess-Martin periodinane (12.0 g, 27.0 mmol) and the reaction stirred at room temperature for 1 h. After completion, the reaction mixture was diluted with saturated aqueous sodium bicarbonate solution (50 mL) and extracted with dichloromethane (2×80 mL). The combined organic layers were dried over anhydrous Na₂SO₄, filtered and concentrated in vacuo to afford tert-butyl (R)-3-(2-oxoethyl)pyrrolidine-1-carboxylate (Intermediate 726, 6.0 g) as a white solid, which was used for the next step without further purification. ¹H NMR (400 MHz, DMSO-d₆) δ ppm 1.37 (s, 9H), 1.90-1.92 (m, 2H), 2.32-2.35 (m, 1H), 2.44-2.46 (m, 1H), 2.55-2.59 (m, 2H), 2.66-2.69 (m, 1H), 2.75-2.82 (m, 1H), 3.28-3.32 (m, 1H), 9.68 (br s, 1H).

### Step 3

### Intermediate 727: tert-butyl (S)-3-(prop-2-yn-1-yl)pyrrolidine-1-carboxylate

To a solution of tert-butyl (R)-3-(2-oxoethyl)pyrrolidine-1-carboxylate (Intermediate 726, 6.0 g, 22.78 mmol) in methanol (50 mL) was added potassium carbonate (6.62 g, 45.56 mmol) followed by dimethyl(1-diazo-2-oxopropyl)phosphonate (6.56 g, 34.17 mmol) and the reaction mixture was stirred at room temperature for 3 h. After completion, the reaction mixture was concentrated in vacuo. The crude material was purified by combi-flash chromatography, by eluting with 40% ethyl acetate in heptane, to afford tert-butyl (S)-3-(prop-2-yn-1-yl)pyrrolidine-1-carboxylate (Intermediate 727, 2 g, 34%) as a gummy liquid. ¹H NMR (400 MHz, DMSO-d₆) δ ppm 1.37 (s, 9H), 1.53-1.65 (m, 1H), 1.89-1.99 (m, 1H), 2.22-2.34 (m, 3H), 2.81-2.85 (m, 1H), 2.88-2.96 (m, 1H), 3.12-3.24 (m, 1H), 3.32-3.37 (m, 1H), 3.33-3.39 (m, 1H).

### Step 4

### Intermediate 728: (S)-3-(prop-2-yn-1-yl)pyrrolidine hydrochloride

To a cooled (0° C.) solution of tert-butyl (S)-3-(prop-2-yn-1-yl)pyrrolidine-1-carboxylate (Intermediate 727, 2.0 g, 9.55 mmol) in 1,4-dioxane (20 mL) was added 4M hydrochloric acid in 1,4-dioxane (50 mL) and the reaction mixture was stirred at room temperature for 3 h. After completion, the reaction mixture was concentrated in vacuo to afford (S)-3-(prop-2-yn-1-yl)pyrrolidine hydrochloride (Intermediate 728, 2.0 g) as a colourless liquid, which was used for the next step without further purification. ¹H NMR (400 MHz, DMSO-d₆), δ ppm 1.57-1.65 (m, 1H), 2.00-2.05 (m, 1H), 2.30-2.41 (m, 3H), 2.74-2.83 (m, 1H), 2.91 (br s, 1H), 3.10-3.14 (m, 1H), 3.15-3.26 (m, 2H), 3.45-3.53 (m, 1H), 9.36 (br s, 1H).

### Step 5

### Intermediate 729: (S)-6-(3-(prop-2-yn-1-yl)pyrrolidin-1-yl)nicotinamide

To a solution of (S)-3-(prop-2-yn-1-yl)pyrrolidine hydrochloride (Intermediate 728, 2.0 g, 18.32 mmol) and 6-chloronicotinamide (CAS No: 6271-78-9, 1.23 g, 7.91 mmol) in dimethylformamide (10 mL) was added potassium carbonate (2.87 g, 18.3 mmol) and the reaction mixture was stirred at room temperature for 3 h. After completion, the reaction mixture was concentrated in vacuo. The crude material was purified by combi-flash chromatography, by eluting with 40% ethyl acetate in heptane, to afford (S)-6-(3-(prop-2-yn-1-yl)pyrrolidin-1-yl)nicotinamide (Intermediate 729, 1.0 g, 23%) as an off-white solid. ¹H NMR (400 MHz, DMSO-d₆) δ ppm 1.74-1.81 (m, 1H), 2.06-2.17 (m, 1H), 2.32-2.37 (m, 2H), 2.42-2.47 (m, 1H), 2.85-2.88 (m, 1H), 3.13-3.19 (m, 1H), 3.37-3.44 (m, 1H), 3.53-3.60 (m, 1H), 3.61-3.69 (m, 1H), 6.43 (d, J=8.93 Hz, 1H), 7.07 (br s, 1H), 7.71 (br s, 1H), 7.93 (dd, J=8.93, 2.45 Hz, 1H), 8.59 (s, 1H). Mass spec m/z 230.14 [M+H]⁺.

### Step 6

### Intermediate 730: 6-((3S)-3-(3-(2-(2,6-dioxo-1-((2-(trimethylsilyl)ethoxy)methyl)piperidin-3-yl)-1-oxoisoindolin-4-yl)prop-2-yn-1-yl)pyrrolidin-1-yl)nicotinamide

To a suspension of (S)-6-(3-(prop-2-yn-1-yl)pyrrolidin-1-yl)nicotinamide (Intermediate 729, 0.50 g, 2.18 mmol) in dimethylformamide (2 mL) was added 3-(4-iodo-1-oxoisoindolin-2-yl)-1-((2-(trimethylsilyl)ethoxy)methyl) piperidine-2,6-dione (Intermediate 8, 1.09 g, 2.18 mmol) followed by triethylamine (10.7 mL, 76.32 mmol). The reaction mixture was purged with argon for 15 min then copper (I)iodide (0.04 g, 0.21 mmol) and PdCl₂(PPh₃)₂ (0.15 g, 0.21 mmol) were added. The reaction mixture was further purged with argon for 10 min and stirred at room temperature for 2 h. After completion, the reaction mixture was diluted with ice cold water (90 mL) and extracted with ethyl acetate (2×80 mL). The combined organic layers were dried over anhydrous Na₂SO₄ and concentrated in vacuo. The crude material was purified by combi-flash chromatography, by eluting with 5% MeOH in DCM, to afford 6-((3S)-3-(3-(2-(2,6-dioxo-1-((2-(trimethylsilyl)ethoxy)methyl)piperidin-3-yl)-1-oxoisoindolin-4-yl)prop-2-yn-1-yl)pyrrolidin-1-yl)nicotinamide (Intermediate 730, 0.5 g, 38%) as a brown solid. Mass spec m/z 602.36 [M+H]⁺.

### Step 7

### Intermediate 731: tert-butyl 6-(6-((3S)-3-(3-(2-(2,6-dioxo-1-((2-(trimethylsilyl)ethoxy)methyl)piperidin-3-yl)-1-oxoisoindolin-4-yl)prop-2-yn-1-yl)pyrrolidin-1-yl)nicotinamido)-2-((R)-1-methylpyrrolidin-2-yl)-1H-pyrrolo[3,2-c]pyridine-1-carboxylate

To a solution of 6-((3S)-3-(3-(2-(2,6-dioxo-1-((2-(trimethylsilyl)ethoxy)methyl)piperidin-3-yl)-1-oxoisoindolin-4-yl)prop-2-yn-1-yl)pyrrolidin-1-yl)nicotinamide (Intermediate 730, 0.5 g, 0.83 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.31 g, 0.83 mmol) followed by cesium carbonate (0.82 g, 2.49 mmol). The reaction mixture was purged with argon for 15 min, then Pd₂(dba)₃ (0.78 g, 0.83 mmol) and Xantphos (0.902 g, 1.47 mmol) were added. The reaction mixture was further purged with argon for 10 min and stirred at 100° C. for 2 h. After completion, the reaction mixture was concentrated in vacuo to obtain the crude compound. The crude material was purified by combi-flash chromatography, by eluting with 5% MeOH in DCM, to afford tert-butyl 6-(6-((3S)-3-(3-(2-(2,6-dioxo-1-((2-(trimethylsilyl)ethoxy)methyl)piperidin-3-yl)-1-oxoisoindolin-4-yl)prop-2-yn-1-yl)pyrrolidin-1-yl)nicotinamido)-2-((R)-1-methylpyrrolidin-2-yl)-1H-pyrrolo[3,2-c]pyridine-1-carboxylate (Intermediate 731, 0.3 g, 42%) as a yellow solid. ¹H NMR (400 MHz, DMSO-d₆) δ ppm −0.05 (s, 9H), 0.78-0.85 (m, 2H), 1.68 (s, 9H), 1.65-1.76 (m, 4H), 2.00-2.12 (m, 1H), 2.16-2.28 (m, 3H), 2.35 (s, 3H), 2.39-2.43 (m, 2H), 2.60-2.77 (m, 4H), 3.02-3.17 (m, 2H), 3.41-3.57 (m, 3H), 3.60-3.70 (m, 1H), 3.69-3.79 (m, 1H), 3.87-3.94 (m, 1H), 4.23-4.33 (m, 1H), 4.43-4.53 (m, 1H), 4.98-5.09 (m, 2H), 5.23-5.28 (m, 1H), 6.50 (d, J=8.80 Hz, 1H), 6.73 (s, 1H), 7.50-7.58 (m, 1H), 7.69 (d, J=7.60 Hz, 1H), 7.74 (d, J=7.60 Hz, 1H), 8.08-8.18 (m, 1H), 8.56 (s, 1H), 8.78-8.80 (m, 1H), 8.90 (s, 1H), 10.41 (br s, 1H). Mass spec m/z 900.6 [M]⁺.

### Step 8

### Example 187: 6-((3S)-3-(3-(2-(2,6-dioxopiperidin-3-yl)-1-oxoisoindolin-4-yl)prop-2-yn-1-yl)pyrrolidin-1-yl)-N-(2-((R)-1-methylpyrrolidin-2-yl)-1H-pyrrolo[3,2-c]pyridin-6-yl)nicotinamide

To a solution of tert-butyl 6-(6-((3S)-3-(3-(2-(2,6-dioxo-1-((2-(trimethylsilyl)ethoxy)methyl)piperidin-3-yl)-1-oxoisoindolin-4-yl)prop-2-yn-1-yl)pyrrolidin-1-yl)nicotinamido)-2-((R)-1-methylpyrrolidin-2-yl)-1H-pyrrolo[3,2-c]pyridine-1-carboxylate (Intermediate 731, 0.30 g, 0.33 mmol) in acetonitrile (5 mL) was added methanesulfonic acid (0.22 mL, 3.32 mmol) and the reaction mixture was stirred at 50° C. for 2 h. The reaction was cooled to room temperature, N,N-dimethylethylenediamine (0.22 mL, 1.99 mmol) and triethylamine (0.93 mL, 6.65 mmol) were added and the reaction stirred at room temperature for 2 h. After completion, the reaction mixture was poured into ice cold water (80 mL), the resultant precipitate was collected by filtration, washed with n-pentane (2×40 mL), and dried in vacuo. The crude material was purified by preparative HPLC (Method B) to afford 6-((3S)-3-(3-(2-(2,6-dioxopiperidin-3-yl)-1-oxoisoindolin-4-yl)prop-2-yn-1-yl)pyrrolidin-1-yl)-N-(2-((R)-1-methylpyrrolidin-2-yl)-1H-pyrrolo[3,2-c]pyridin-6-yl)nicotinamide (Example 187, 0.18 g, 8%) as an off-white solid. ¹H NMR (400 MHz, DMSO-d₆) δ ppm 1.83-1.93 (m, 4H), 1.99-2.02 (m, 1H), 2.19-2.42 (m, 7H), 2.42-2.46 (m, 3H), 2.59-2.64 (m, 1H), 2.68-2.72 (m, 2H), 2.87-2.95 (m, 1H), 3.13-3.16 (m, 1H), 3.45-3.52 (m, 1H), 3.61-3.70 (m, 1H), 3.71-3.79 (m, 1H), 4.29-4.37 (m, 1H), 4.44-4.48 (m, 1H), 5.09-5.14 (m, 1H), 6.37 (s, 1H), 6.49-6.59 (m, 1H), 7.50-7.56 (m, 1H), 7.67 (d, J=7.13 Hz, 1H), 7.72 (d, J=7.63 Hz, 1H), 8.11-8.17 (m, 2H), 8.47 (s, 1H), 8.78 (s, 1H), 10.18 (br s, 1H), 10.98 (br s, 1H), 11.31 (br s, 1H). Mass spec m/z 671.2 [M+H]⁺.

### Example 188 was Synthesised Following Scheme 181

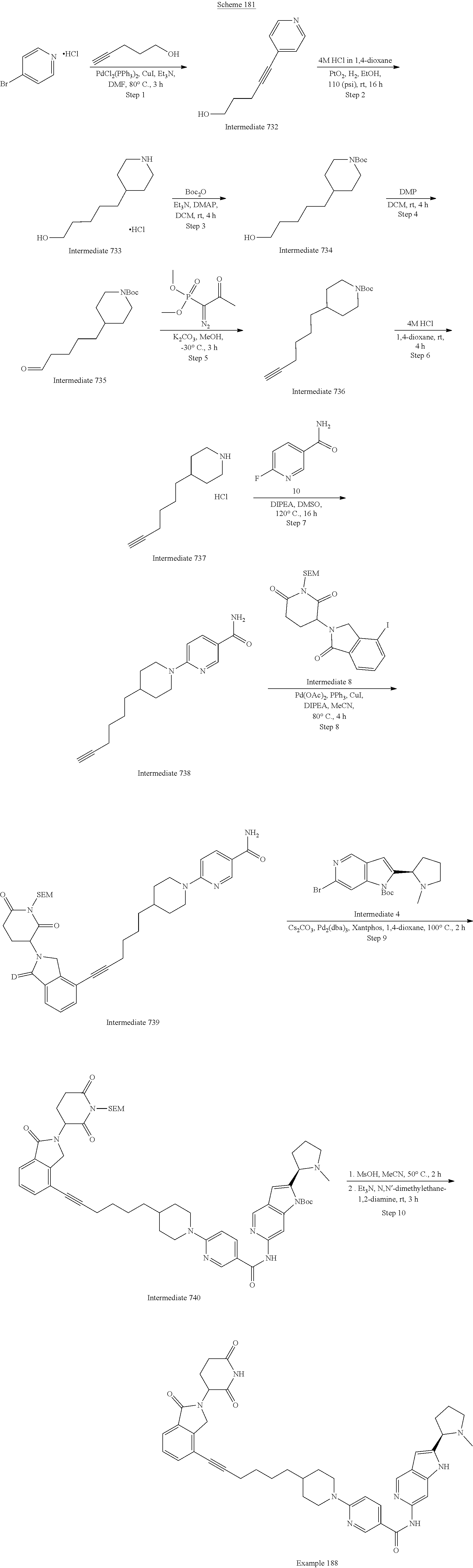

### Step 1

### Intermediate 732: 5-(pyridin-4-yl)pent-4-yn-1-ol

To a solution of 4-bromopyridine hydrochloride (CAS No: 19524-06-2, 18.0 g, 91.61 mmol) and pent-4-yn-1-ol (CAS No. 5390-04-5, 11.56 g, 137.43 mmol) in dimethylformamide (50 mL) was added triethylamine (200 mL, 1430 mmol). The reaction mixture was purged with argon for 15 min then PdCl₂(PPh₃)₂ (6.49 g, 9.16 mmol) and copper (I) iodide (1.78 g, 9.16 mmol) were added. The reaction mixture was purged with argon for another 10 min and stirred at 80° C. for 3 h. After completion, the reaction mixture was concentrated in vacuo, diluted with water (500 mL) and extracted with ethyl acetate (3×200 mL). The combined organic layers were dried over anhydrous Na₂SO₄, filtered and concentrated in vacuo. The crude material was purified by combi-flash chromatography, by eluting with 80% ethyl acetate in heptane, to afford 5-(pyridin-4-yl)pent-4-yn-1-ol (Intermediate 732, 14.76 g, 95%) as a yellow solid. ¹H NMR (400 MHz, DMSO-d₆) δ ppm 1.65-1.72 (m, 2H), 2.48-2.52 (m, 2H), 3.40-3.53 (m, 2H), 4.53-4.56 (m, 1H), 7.34 (d, J=5.6 Hz, 2H), 8.52 (d, J=4.8 Hz, 2H). Mass spec m/z 162.5 [M+H]⁺.

### Step 2

### Intermediate 733: 5-(piperidin-4-yl)pentan-1-ol hydrochloride

To a solution of 5-(pyridin-4-yl)pent-4-yn-1-ol (Intermediate 732, 7.0 g, 43.4 mmol) in 1,4-dioxane (70 mL) was added 4M hydrochloric acid in 1,4-dioxane (70 mL) at 0° C. and the reaction stirred at room temperature for 30 min. The reaction mixture was concentrated in vacuo and dissolved in ethanol (100 mL) followed by the addition of platinum(IV) oxide (2.96 g, 13.0 mmol) at room temperature. The reaction mixture was stirred at room temperature for 16 h under hydrogen atmosphere at 110 psi. After completion, the reaction mixture was filtered through a celite bed, the filter pad was washed with methanol (100 mL) and the filtrate concentrated in vacuo to afford 5-(piperidin-4-yl)pentan-1-ol hydrochloride (Intermediate 733, 7.0 g, 94%) as a colorless liquid which was used for the next step without further purification. ¹H NMR (400 MHz, DMSO-d₆) δ ppm 1.12-1.15 (m, 12H), 1.65-1.75 (m, 2H), 1.67-1.77 (m, 2H), 3.15-3.25 (m, 2H), 3.40-3.50 (m, 2H), 4.36-4.42 (m, 1H).

### Step 3

### Intermediate 734: tert-butyl 4-(5-hydroxypentyl)piperidine-1-carboxylate

To a cooled (0° C.) solution of 5-(piperidin-4-yl)pentan-1-ol (Intermediate 733, 14 g, 81.74 mmol) in dichloromethane (150 mL) was added triethylamine (45.8 mL, 326.9 mmol), di-tert-butyl dicarbonate (37.9 g, 163.48 mmol) and 4-dimethylaminopyridine (2.01 g, 16.3 mmol) and the reaction mixture was stirred at room temperature for 4 h. After completion, the reaction mixture was diluted with water (200 mL) and extracted with dichloromethane (3×200 mL). The combined organic layers were dried over anhydrous Na₂SO₄, filtered and concentrated in vacuo. The crude material was purified by combi-flash chromatography, by eluting with 45% ethyl acetate in heptane, to afford tert-butyl 4-(5-hydroxypentyl)piperidine-1-carboxylate (Intermediate 734, 6.8 g, 31%) as a yellow liquid. ¹H NMR (400 MHz, DMSO-d₆) δ ppm 0.85-0.97 (m, 2H), 1.07-1.20 (m, 6H), 1.45-1.53 (m, 12H), 1.58-1.63 (m, 2H), 2.58-2.62 (m, 2H), 3.34-3.39 (m, 2H), 3.88-3.98 (m, 2H), 4.30-4.33 (m, 1H). Mass spec m/z 272.4 [M−H]⁻.

### Step 4

### Intermediate 735: tert-butyl 4-(5-oxopentyl)piperidine-1-carboxylate

To a cooled (0° C.) solution of tert-butyl 4-(5-hydroxypentyl)piperidine-1-carboxylate (Intermediate 734, 5.70 g, 21.0 mmol) in dichloromethane (60 mL) was added was Dess-Martin periodinane (13.57 g, 32.0 mmol) and the reaction mixture was stirred at room temperature for 4 h. After completion, the reaction mixture was diluted with sodium bicarbonate (300 mL) and extracted with dichloromethane (3×200 mL). The combined organic layers were dried over anhydrous Na₂SO₄, filtered and concentrated in vacuo. The crude material was purified by combi-flash chromatography, by eluting with 20% ethyl acetate in heptane, to afford tert-butyl 4-(5-oxopentyl)piperidine-1-carboxylate (Intermediate 735, 4.0 g, 71%) as a yellow liquid. ¹H NMR (400 MHz, DMSO-d₆) δ ppm 0.85-0.97 (m, 2H), 1.13-1.28 (m, 5H), 1.38 (s, 9H), 1.43-1.53 (m, 2H), 1.59-1.62 (m, 2H), 2.40-2.44 (m, 2H), 2.58-2.71 (m, 2H), 3.89-3.97 (m, 2H), 9.66 (s, 1H).

### Step 5

### Intermediate 736: tert-butyl 4-(hex-5-yn-1-yl)piperidine-1-carboxylate

To a cooled (−30° C.) solution of tert-butyl 4-(5-oxopentyl)piperidine-1-carboxylate (Intermediate 735, 4.0 g, 14.85 mmol) in methanol (50 mL) was added potassium carbonate (4.10 g, 29.7 mmol) and 1-diazo-1-dimethoxyphosphoryl-propan-2-one (4.28 g, 22.27 mmol) dropwise and the reaction stirred at −30° C. for 3 h. After completion, the reaction mixture was concentrated in vacuo, diluted with water (500 mL) and extracted with diethyl ether (3×200 mL). The combined organic layers were dried over anhydrous Na₂SO₄, filtered and concentrated in vacuo. The crude material was purified by combi-flash chromatography, by eluting with 10% ethyl acetate in heptane, to afford tert-butyl 4-(hex-5-yn-1-yl)piperidine-1-carboxylate (Intermediate 736, 3.7 g, 94%) as a yellow liquid. ¹H NMR (400 MHz, DMSO-d₆) δ ppm 0.91-0.99 (m, 2H), 1.17-1.24 (m, 17H), 1.59-1.62 (s, 2H), 2.13-2.15 (m, 2H), 2.62-2.73 (m, 2H), 3.98-3.91 (m, 2H).

### Step 6

### Intermediate 737: 4-(hex-5-yn-1-yl)piperidine hydrochloride

To a cooled (0° C.) solution of tert-butyl 4-(hex-5-yn-1-yl)piperidine-1-carboxylate (Intermediate 736, 2.50 g, 9.91 mmol) in 1,4-dioxane (15 mL) was added 4M hydrochloric acid in 1,4-dioxane (25.0 mL, 100.0 mmol) and the reaction mixture was stirred at room temperature for 4 h. After completion, the reaction mixture was concentrated in vacuo to afford 4-(hex-5-yn-1-yl)piperidine hydrochloride (Intermediate 737, 5.0 g) as a white solid, which was used for next step without further purification. ¹H NMR (400 MHz, DMSO-d₆) δ ppm 1.04-1.46 (m, 9H), 1.72-1.76 (m, 2H), 2.13-2.16 (m, 2H), 2.50-2.81 (m, 2H), 3.17-3.20 (m, 2H), 3.15-3.59 (m, 2H).

### Step 7

### Intermediate 738: 6-(4-(hex-5-yn-1-yl)piperidin-1-yl)nicotinamide

To a solution of 4-(hex-5-yn-1-yl)piperidine (Intermediate 737, 1.0 g, 6.05 mmol) in dimethylformamide (10 mL) were added 6-fluoronicotinamide (CAS No: 369-50-6, 1.02 g, 7.26 mmol) and potassium carbonate (3.34 g, 24.2 mmol) and the reaction mixture was heated at 120° C. for 16 h. After completion, the reaction mixture was quenched with ice cold water (50 mL) and extracted with diethyl ether (2×50 mL). The combined organic layers were dried over anhydrous Na₂SO₄, filtered and concentrated in vacuo. The crude material was purified by combi-flash chromatography, by eluting with 50% ethyl acetate in heptane, to afford 6-(4-(hex-5-yn-1-yl)piperidin-1-yl)nicotinamide (Intermediate 738, 0.75 g, 42%) as an off-white solid. ¹H NMR (400 MHz, DMSO-d₆) δ ppm 1.01-1.09 (m, 2H), 1.20-1.24 (m, 2H), 1.42-1.51 (m, 5H), 1.69-1.72 (m, 2H), 2.14-2.16 (m, 2H), 2.74 (s, 1H), 2.79-2.86 (m, 2H), 4.37-4.40 (m, 2H), 6.80 (d, J=8.8 Hz, 1H), 7.07 (bs, 1H), 7.70 (bs, 1H), 7.91 (d, J=8.8 Hz, 1H), 8.58 (s, 1H). Mass spec m/z 286.4 [M+H]⁺.

### Step 8

### Intermediate 739: 6-(4-(6-(2-(2,6-dioxo-1-((2-(trimethylsilyl)ethoxy)methyl)piperidin-3-yl)-1-oxoisoindolin-4-yl)hex-5-yn-1-yl)piperidin-1-yl)nicotinamide

To a solution of 3-(4-iodo-1-oxoisoindolin-2-yl)-1-((2-(trimethylsilyl)ethoxy)methyl)piperidine-2,6-dione (Intermediate 8, 1.23 g, 2.45 mmol) and 6-(4-(hex-5-yn-1-yl)piperidin-1-yl)nicotinamide (Intermediate 738, 0.70 g, 2.45 mmol) in acetonitrile (20 mL) was added N,N-diisopropylethylamine (2.0 mL, 12.26 mmol). The reaction mixture was purged with argon for 15 min then Pd(OAc)₂ (0.06 g, 0.36 mmol) and copper(I) iodide (0.05 g, 0.24 mmol) were added. The reaction mixture was further purged with argon for 10 min and heated at 80° C. for 4 h. After completion, the reaction mixture was diluted with water (100 mL) and extracted with ethyl acetate (3×50 mL). The combined organic layers were dried over anhydrous Na₂SO₄, filtered and concentrated in vacuo. The crude material was purified by combi-flash chromatography, by eluting with 2% MeOH in DCM, to afford 6-(4-(6-(2-(2,6-dioxo-1-((2-(trimethylsilyl)ethoxy)methyl)piperidin-3-yl)-1-oxoisoindolin-4-yl)hex-5-yn-1-yl)piperidin-1-yl)nicotinamide (Intermediate 739, 1.5 g, 89%) as a yellow solid. ¹H NMR (400 MHz, DMSO-d₆) δ ppm −0.81 (s, 9H), 0.81-0.86 (m, 2H), 1.04-1.11 (m, 3H), 1.46-1.59 (m, 4H), 1.71-1.74 (m, 2H), 2.06-2.12 (m, 1H), 2.79-2.85 (m, 2H), 3.04-3.18 (m, 4H), 3.49-3.65 (m, 4H) 4.24-4.50 (m, 4H), 5.01-5.09 (m, 2H), 5.27-5.32 (m, 1H), 6.80 (d, J=9.2 Hz, 1H), 7.11 (s, 1H), 7.54 (t, J=8.0 Hz, 1H), 7.62-7.66 (m, 1H), 7.73 (dd, J=7.2 Hz, 2H), 7.92 (dd, J=8.8, 2.4 Hz, 1H), 8.17 (s, 1H), 8.58 (s, 1H). Mass spec m/z 658.1 [M+H]⁺.

### Step 9

### Intermediate 740: tert-butyl 6-(6-(4-(6-(2-(2,6-dioxo-1-((2-(trimethylsilyl)ethoxy)methyl)piperidin-3-yl)-1-oxoisoindolin-4-yl)hex-5-yn-1-yl)piperidin-1-yl)nicotinamido)-2-((R)-1-methylpyrrolidin-2-yl)-1H-pyrrolo[3,2-c]pyridine-1-carboxylate

To a solution of 6-(4-(6-(2-(2,6-dioxo-1-((2-(trimethylsilyl)ethoxy)methyl)piperidin-3-yl)-1-oxoisoindolin-4-yl)hex-5-yn-1-yl)piperidin-1-yl)nicotinamide (Intermediate 739, 0.50 g, 0.76 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.35 g, 0.91 mmol) and cesium carbonate (0.37 g, 0.23 mmol). The reaction mixture was purged with argon for 15 min then Xanthphos (0.13 g, 0.23 mmol) and Pd₂(dba)₃ (0.11 g, 0.11 mmol) were added. The reaction mixture was further purged with argon for 10 min and heated at 100° C. for 2 h. The reaction mixture was then filtered through a celite bed and the filter pad washed with ethyl acetate (100 mL). The filtrate was concentrated in vacuo and the crude material purified by combi-flash chromatography, by eluting with 70% ethyl acetate in heptane, to afford tert-butyl 6-(6-(4-(6-(2-(2,6-dioxo-1-((2-(trimethylsilyl)ethoxy)methyl)piperidin-3-yl)-1-oxoisoindolin-4-yl)hex-5-yn-1-yl)piperidin-1-yl)nicotinamido)-2-((R)-1-methylpyrrolidin-2-yl)-1H-pyrrolo[3,2-c]pyridine-1-carboxylate (Intermediate 740, 0.54 g, 59%) as a yellow solid. ¹H NMR (400 MHz, DMSO-d₆) δ ppm −0.31 (s, 9H), 0.82-0.86 (m, 2H), 1.07-1.11 (m, 4H), 1.24-1.28 (m, 3H), 1.47-1.76 (m, 18H), 2.32-2.43 (m, 5H), 2.83-2.90 (m, 3H), 3.03-3.16 (m, 2H), 3.36-3.41 (m, 2H) 3.49-3.55 (m, 2H), 3.89-3.91 (m, 1H), 4.25-4.29 (m, 1H), 4.41-4.49 (m, 3H), 5.01-5.09 (m, 2H), 5.26-5.32 (m, 1H), 6.73 (s, 1H), 6.84 (d, J=9.2 Hz, 1H), 7.53 (t, J=8.8 Hz, 1H), 7.64 (m, J=7.2 Hz, 1H), 7.72 (d, J=7.2 Hz, 1H), 8.12 (d, J=8.8 Hz, 1H), 8.56 (s, 1H), 8.77 (s, 1H), 8.89 (s, 1H), 10.44 (s, 1H). Mass spec m/z 956.7 [M−H]⁻.

### Step 10

### Example 188: 6-(4-(6-(2-(2,6-dioxopiperidin-3-yl)-1-oxoisoindolin-4-yl)hex-5-yn-1-yl)piperidin-1-yl)-N-(2-((R)-1-methylpyrrolidin-2-yl)-1H-pyrrolo[3,2-c]pyridin-6-yl)nicotinamide

To a solution of tert-butyl 6-(6-(4-(6-(2-(2,6-dioxo-1-((2-(trimethylsilyl)ethoxy)methyl)piperidin-3-yl)-1-oxoisoindolin-4-yl)hex-5-yn-1-yl)piperidin-1-yl)nicotinamido)-2-((R)-1-methylpyrrolidin-2-yl)-1H-pyrrolo[3,2-c]pyridine-1-carboxylate (Intermediate 740, 0.54 g, 0.56 mmol) in acetonitrile (7 mL) was added methanesulfonic acid (0.37 mL, 5.6 mmol) and the reaction mixture was heated at 50° C. for 2 h. After cooling to room temperature, N,N′-dimethylethylenediamine (0.34 g, 2.8 mmol) followed by triethylamine (1.58 mL, 11.28 mmol) were added and the reaction mixture was stirred at room temperature for 3 h. The reaction mixture was then diluted with water (50 mL), the resultant precipitate was collected by filtration and dried in vacuo. The crude material was purified by preparative HPLC (Method A) to afford 6-(4-(6-(2-(2,6-dioxopiperidin-3-yl)-1-oxoisoindolin-4-yl)hex-5-yn-1-yl)piperidin-1-yl)-N-(2-((R)-1-methylpyrrolidin-2-yl)-1H-pyrrolo[3,2-c]pyridin-6-yl)nicotinamide (Example 188, 0.067 g, 15%) as an off-white solid. ¹H NMR (400 MHz, DMSO-d₆) δ ppm 1.05-1.12 (m, 2H), 1.21-1.31 (m, 3H), 1.45-1.60 (m, 4H), 1.73-1.95 (m, 4H), 2.00-2.08 (m, 1H), 2.12-2.18 (m, 4H), 2.21-2.30 (m, 1H), 2.41-2.48 (m, 3H), 2.56-2.68 (m, 1H), 2.73-2.95 (m, 3H), 3.11-3.16 (m, 1H), 3.28-3.30 (m, 2H), 4.28-4.33 (m, 1H), 4.41-4.78 (m, 3H), 5.12-5.17 (m, 1H), 6.37 (s, 1H), 6.84 (d, J=9.2 Hz, 1H), 7.52 (t, J=7.6 Hz, 1H), 7.63 (m, J=7.2 Hz, 1H), 7.10 (d, J=7.6 Hz, 1H), 8.11-8.16 (m, 2H), 8.47 (s, 1H), 8.77 (s, 1H), 10.23 (s, 1H), 11.01 (s, 1H), 11.33 (s, 1H). Mass spec m/z 727.3 [M+H]⁺.

### Example 189 was Synthesised Following Scheme 182

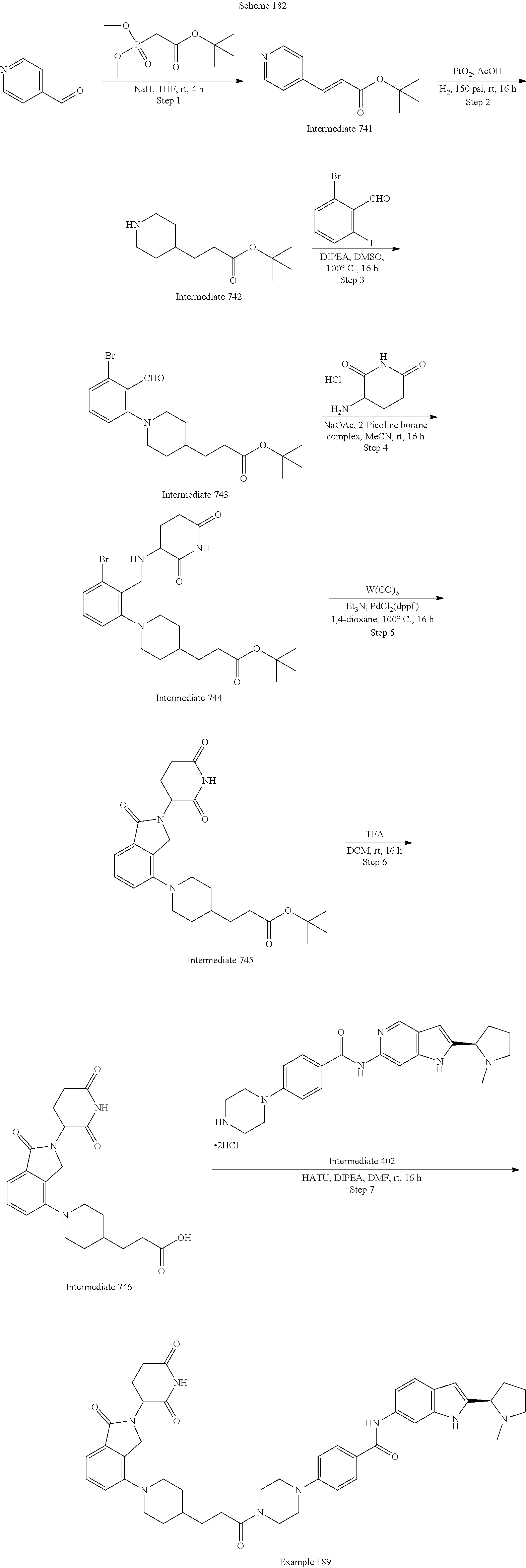

### Step 1

### Intermediate 741: tert-butyl (E)-3-(pyridin-4-yl)acrylate

To a solution of tert-butyl 2-(dimethoxyphosphoryl)acetate (CAS No: 62327-21-3, 5.0 g, 22.30 mmol) in tetrahydrofuran (50 mL) was added isonicotinaldehyde (CAS No: 872-85-5, 2.38 g, 22.30 mmol), then sodium hydride (60% in mineral oil, 0.89 g, 22.30 mmol) and the reaction stirred at room temperature for 4 h. After completion, the reaction mixture was quenched with saturated aqueous ammonium chloride solution (100 mL) and extracted with ethyl acetate (200 mL). The combined organic layers were dried over anhydrous Na₂SO₄, filtered, and concentrated in vacuo. The crude material was purified by combi-flash chromatography, by eluting with 40% ethyl acetate in heptane, to afford tert-butyl (E)-3-(pyridin-4-yl)acrylate (Intermediate 741, 3.75 g, 76%) as a yellow liquid. ¹H NMR (400 MHz, DMSO-d₆) δ ppm 1.49 (s, 9H), 6.77 (d, J=16.0 Hz, 1H), 7.52 (d, J=16.0 Hz, 1H), 7.65-7.66 (m, 2H), 8.60-8.61 (m, 2H). Mass spec m/z 206.1 [M+H]⁺.

### Step 2

### Intermediate 742: tert-butyl 3-(piperidin-4-yl)propanoate

To a solution of tert-butyl (E)-3-(pyridin-4-yl)acrylate (Intermediate 741, 7.50 g, 37.0 mmol) in acetic acid (100 mL) was added platinum (IV) oxide (2.5 g, 11.0 mmol) and the reaction mixture was stirred at room temperature for 16 h under hydrogen atmosphere at 150 psi. After completion, the reaction mixture was filtered through a celite bed and the filter pad was washed with ethyl acetate (50 mL). The filtrate was concentrated in vacuo to afford tert-butyl 3-(piperidin-4-yl)propanoate (Intermediate 742, 8.0 g, 95%) as a colourless liquid, which was used for the next step without further purification. ¹H NMR (400 MHz, DMSO-d₆) δ ppm 1.11-1.20 (m, 2H), 1.39-1.45 (m, 13H), 1.65-1.68 (s, 2H), 2.18-2.22 (m, 2H), 2.58-2.64 (m, 2H), 3.06-3.09 (m, 2H). Mass spec m/z 214.2 [M+H]⁺.

### Step 3

### Intermediate 743: tert-butyl 3-(1-(3-bromo-2-formylphenyl)piperidin-4-yl)propanoate

To a solution of tert-butyl 3-(piperidin-4-yl)propanoate (Intermediate 742, 8.0 g, 37.50 mmol) in dimethyl sulfoxide (80 mL) were added 2-bromo-6-fluorobenzaldehyde (CAS No: 360575-28-6, 7.61 g, 37.50 mmol) and N,N-diisopropylethylamine (39.30 mL, 225.0 mmol) and the reaction heated at 100° C. for 16 h. After completion, the reaction mixture was quenched with water (100 mL) and extracted with ethyl acetate (300 mL). The combined organic layers were dried over anhydrous Na₂SO₄, filtered, and concentrated in vacuo to afford tert-butyl 3-(1-(3-bromo-2-formylphenyl)piperidin-4-yl)propanoate (Intermediate 743, 8.2 g, 55%) as a yellow solid, which was used for the next step without further purification. ¹H NMR (400 MHz, DMSO-d₆) δ ppm 1.29-1.41 (m, 12H), 1.47-1.52 (m, 2H), 1.71-1.74 (s, 2H), 2.22-2.26 (m, 2H), 2.78-2.83 (m, 2H), 3.14-3.17 (m, 2H), 7.21 (d, J=8.0 Hz, 1H), 7.28 (d, J=7.6 Hz, 1H), 7.40 (t, J=8.0 Hz, 1H), 10.02 (s, 1H). Mass spec m/z 398 [M+H+2]⁺.

### Step 4

### Intermediate 744: tert-butyl 3-(1-(3-bromo-2-(((2,6-dioxopiperidin-3-yl)amino)methyl)phenyl)piperidin-4-yl)propanoate

To a solution of tert-butyl 3-(1-(3-bromo-2-formylphenyl)piperidin-4-yl)propanoate (Intermediate 743, 5.0 g, 12.62 mmol) and 3-aminopiperidine-2,6-dione hydrochloride (CAS No: 24666-56-6, 2.07 g, 12.62 mmol)) in acetonitrile (50 mL) were added sodium acetate (1.25 g, 15.14 mmol) and 2-picolineborane complex (4.26 g, 37.85 mmol) and the reaction mixture was stirred at room temperature for 16 h. After completion, the reaction mixture was filtered and the filtrate was evaporated in vacuo. The crude material was purified by combi-flash chromatography, by eluting with 80% ethyl acetate in heptane, to afford tert-butyl 3-(1-(3-bromo-2-(((2,6-dioxopiperidin-3-yl)amino)methyl)phenyl)piperidin-4-yl)propanoate (Intermediate 744, 3.0 g, 45%) as a pale blue solid. ¹H NMR (400 MHz, DMSO-d₆) δ ppm 1.21-1.58 (m, 13H), 1.63-1.77 (m, 3H), 2.11-2.32 (m, 3H), 2.52-2.58 (m, 3H), 2.61-2.73 (m, 1H), 2.81-2.95 (m, 1H), 3.03-3.09 (m, 1H), 3.13-3.25 (m, 2H), 3.87-3.94 (m, 2H), 4.14-4.17 (m, 1H), 7.11-7.16 (m, 1H), 7.25-7.35 (m, 2H), 10.76 (s, 1H). Mass spec 510.1 [M+H+2]⁺.

### Step 5

### Intermediate 745: tert-butyl 3-(1-(2-(2,6-dioxopiperidin-3-yl)-1-oxoisoindolin-4-yl)piperidin-4-yl)propanoate

To a solution of tert-butyl 3-(1-(3-bromo-2-(((2,6-dioxopiperidin-3-yl)amino)methyl)phenyl)piperidin-4-yl)propanoate (Intermediate 744, 3.0 g, 5.90 mmol) in 1,4-dioxane (30 mL) was added triethylamine (2.48 mL 17.70 mmol). The reaction mixture was purged with argon for 15 min then Xantphos (0.70 g, 1.18 mmol) followed by PdCl₂(dppf) (0.90 g, 1.18 mmol) and W(CO)₆ (1.07 g, 2.95 mmol) were added. The reaction mixture was further purged with argon for 10 min and heated at 100° C. for 16 h. After completion, the reaction mixture was filtered through a celite bed and the filter pad was washed with ethyl acetate (200 mL). The filtrate was concentrated in vacuo and the crude material purified by combi-flash chromatography, by eluting with ethyl acetate, to afford of tert-butyl 3-(1-(2-(2,6-dioxopiperidin-3-yl)-1-oxoisoindolin-4-yl)piperidin-4-yl)propanoate (Intermediate 745, 2.5 g, 93%) as a yellow solid. ¹H NMR (400 MHz, DMSO-d₆) δ ppm 1.23-1.50 (m, 16H), 1.59-1.77 (m, 2H), 2.22-2.25 (m, 2H), 2.52-2.75 (m, 4H), 2.85-2.96 (m, 1H), 3.36-3.39 (m, 1H), 4.26-4.44 (m, 2H), 5.10-5.12 (m, 1H), 7.14-7.16 (m, 1H), 7.28-7.41 (m, 1H), 7.48-7.62 (m, 1H), 10.95-11.13 (s, 1H). Mass spec m/z 456.2 [M+H]⁺.

### Step 6

### Intermediate 746: 3-(1-(2-(2,6-dioxopiperidin-3-yl)-1-oxoisoindolin-4-yl)piperidin-4-yl)propanoic acid

To a cooled (0° C.) solution of tert-butyl 3-(1-(2-(2,6-dioxopiperidin-3-yl)-1-oxoisoindolin-4-yl)piperidin-4-yl)propanoate (Intermediate 745, 2.0 g, 4.39 mmol) in dichloromethane (20 mL) was added trifluoroacetic acid (7.0 mL, 12.96 mmol) and the reaction mixture was stirred at room temperature for 16 h. After completion, the reaction mixture was concentrated in vacuo to afford 3-(1-(2-(2,6-dioxopiperidin-3-yl)-1-oxoisoindolin-4-yl)piperidin-4-yl)propanoic acid (Intermediate 746, 1.5 g, 86%) as a white solid, which was used for the next step without further purification. ¹H NMR (400 MHz, DMSO-d₆) δ ppm 1.24-1.53 (m, 5H), 1.76-1.79 (m, 2H), 1.99-2.02 (m, 2H), 2.26-2.33 (m, 2H), 2.51-2.75 (m, 3H), 2.87-2.92 (m, 1H), 3.37-3.42 (m, 2H), 4.28-4.33 (m, 1H), 4.39-4.56 (m, 1H), 5.11-5.13 (m, 1H), 7.18-7.21 (m, 1H), 7.30-7.33 (m, 1H), 7.41-7.44 (m, 1H), 10.97-10.99 (s, 1H). Mass spec m/z 400.1 [M+H]⁺.

### Step 7

### Example 189: 4-(4-(3-(1-(2-(2,6-dioxopiperidin-3-yl)-1-oxoisoindolin-4-yl)piperidin-4-yl)propanoyl)piperazin-1-yl)-N-(2-((R)-1-methylpyrrolidin-2-yl)-1H-pyrrolo[3,2-c]pyridin-6-yl)benzamide

To a solution of 3-(1-(2-(2,6-dioxopiperidin-3-yl)-1-oxoisoindolin-4-yl)piperidin-4-yl)propanoic acid (Intermediate 746, 0.40 g, 1.00 mmol) in dimethylformamide (4 mL) was added HATU (0.58 g, 1.50 mmol) and N,N-diisopropylethylamine (0.87 mL, 5.00 mmol) and the reaction mixture was stirred at room temperature for 15 min. (R)—N-(2-(1-methylpyrrolidin-2-yl)-1H-pyrrolo[3,2-c]pyridin-6-yl)-4-(piperazin-1-yl)benzamide dihydrochloride (Intermediate 402, 0.44 g, 1.10 mmol) was then added and the reaction mixture was stirred at room temperature for 16 h. After completion, the reaction mixture poured into ice cold water (30 mL), resulting in a solid precipitate, which was collected by filtration and dried in vacuo. The crude material was purified by preparative HPLC (Method A) to afford 4-(4-(3-(1-(2-(2,6-dioxopiperidin-3-yl)-1-oxoisoindolin-4-yl)piperidin-4-yl)propanoyl)piperazin-1-yl)-N-(2-((R)-1-methylpyrrolidin-2-yl)-1H-pyrrolo[3,2-c]pyridin-6-yl)benzamide (Example 189, 0.02 g, 3%) as an off-white solid. ¹H NMR (400 MHz, DMSO-d₆) δ ppm 1.24-1.35 (m, 2H), 1.38-1.46 (m, 1H), 1.51-1.56 (m, 2H), 1.77-2.03 (m, 6H), 2.12-2.19 (m, 4H), 2.23-2.28 (m, 2H), 2.39-2.46 (m, 3H), 2.52-2.63 (m, 3H), 2.78-2.86 (m, 2H), 2.88-2.93 (m, 1H), 3.12-3.19 (m, 1H), 3.33-3.41 (m, 4H), 3.62-3.67 (m, 4H), 4.29-4.31 (m, 1H), 4.41-4.48 (m, 1H), 5.08-5.13 (m, 1H), 6.37 (s, 1H), 7.00 (d, J=9.2 Hz, 2H), 7.16 (d, J=7.6 Hz, 1H), 7.29 (d, J=7.6 Hz, 1H), 7.42 (t, J=7.6 Hz, 1H), 7.98 (d, J=7.6 Hz, 2H), 8.17 (s, 1H), 8.48 (s, 1H), 10.11 (s, 1H), 10.97 (s, 1H), 11.31 (s, 1H). Mass spec m/z 786.3 [M+H]⁺.

### Example 190 was Synthesised Following Scheme 183

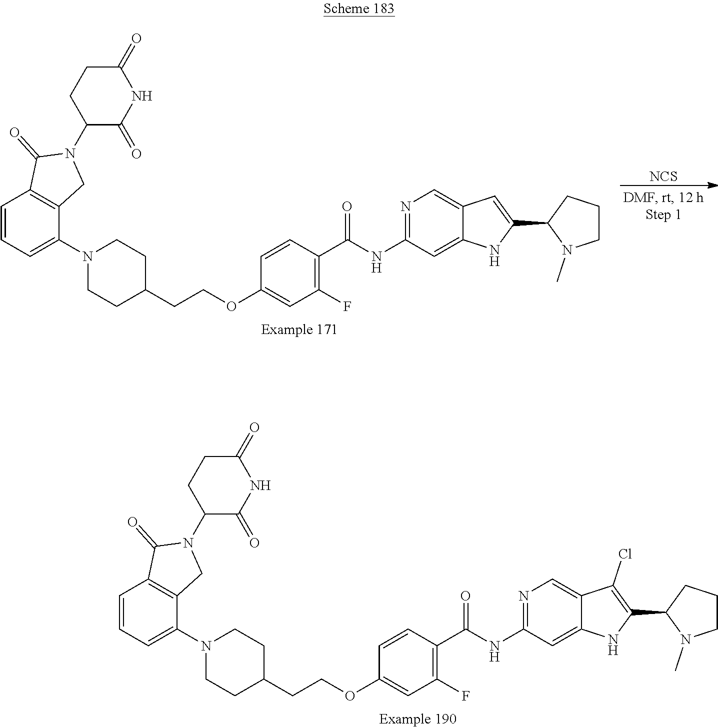 [SMILES: CN1CCC[C@@H]1c1cc2cnc(NC(=O)c3ccc(OCCC4CCN(c5cccc6c5CN(C5CCC(=O)NC5=O)C6=O)CC4)cc3F)cc2[nH]1 ; "NCS" ; CN1CCC[C@@H]1c1[nH]c2cc(NC(=O)c3ccc(OCCC4CCN(c5cccc6c5CN(C5CCC(=O)NC5=O)C6=O)CC4)cc3F)ncc2c1Cl]

### Step 1

### Example 190: N-(3-chloro-2-((R)-1-methylpyrrolidin-2-yl)-1H-pyrrolo[3,2-c]pyridin-6-yl)-4-(2-(1-(2-(2,6-dioxopiperidin-3-yl)-1-oxoisoindolin-4-yl)piperidin-4-yl)ethoxy)-2-fluorobenzamide

To solution of 4-(2-(1-(2-(2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide (Example 171, 0.10 g, 0.12 mmol) in dimethylformamide (3 mL) was added N-chlorosuccinimide (0.05 g, 0.37 mmol) and the reaction mixture was stirred at room temperature for 12 h. The reaction mixture was then diluted with ice cold water (100 mL), the resultant precipitate was collected by filtration and dried in vacuo. The crude material was purified by preparative HPLC (Method A) to afford N-(3-chloro-2-((R)-1-methylpyrrolidin-2-yl)-1H-pyrrolo[3,2-c]pyridin-6-yl)-4-(2-(1-(2-(2,6-dioxopiperidin-3-yl)-1-oxoisoindolin-4-yl)piperidin-4-yl)ethoxy)-2-fluorobenzamide (Example 190, 0.023 g, 23%) as an off-white solid. ¹H NMR (400 MHz, DMSO-d₆) δ ppm 1.32-1.45 (m, 2H), 1.60-1.7 (m, 1H), 1.71-1.8 (m, 2H), 1.81-1.91 (m, 4H), 1.98-2.03 (m, 2H), 2.16-2.20 (m, 4H), 2.25-2.31 (m, 1H), 2.55-2.63 (m, 2H), 2.70-2.8 (m, 2H), 2.81-2.95 (m, 3H), 3.15-3.18 (m, 1H), 3.48-3.51 (m, 1H), 4.14-4.17 (m, 2H), 4.24-4.46 (m, 2H), 5.09-5.13 (m, 1H), 6.85-6.9 (m, 1H), 6.96-7.0 (m, 1H), 7.17 (d, J=8.0 Hz, 1H), 7.30 (d, J=7.6 Hz, 1H), 7.43 (t, J=7.6 Hz, 1H), 7.74 (t, J=8.8 Hz, 1H), 8.22 (s, 1H), 8.46 (s, 1H), 10.15 (br s, 1H), 10.96 (br s, 1H), 11.7 (s, 1H). Mass spec m/z 742.3 [M+H]⁺.

### Example 191 was Synthesised Following Scheme 184

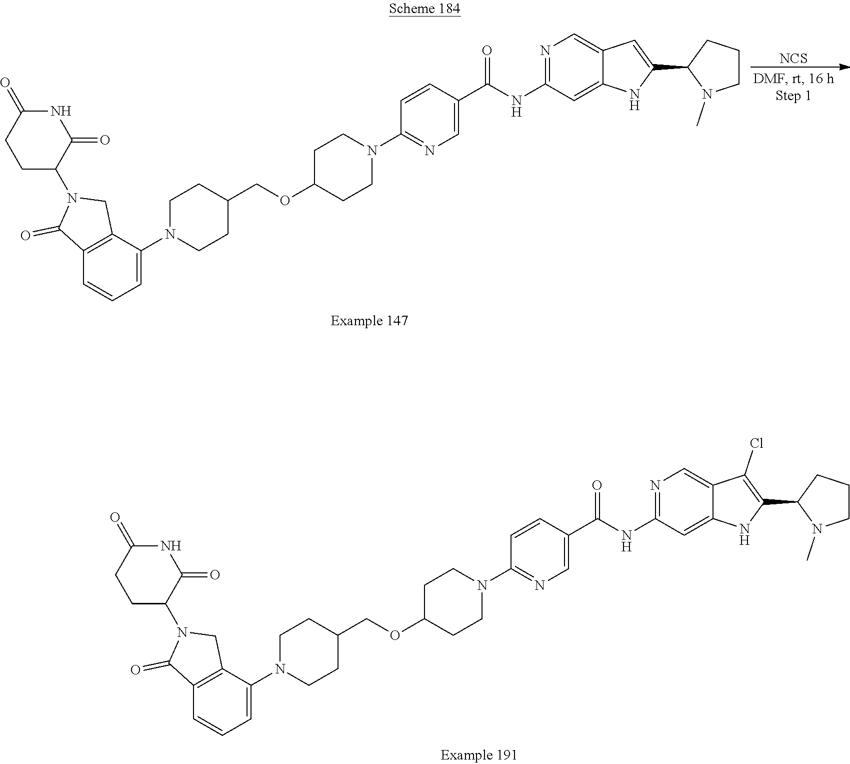

### Step 1

### Example 191: N-(3-chloro-2-((R)-1-methylpyrrolidin-2-yl)-1H-pyrrolo[3,2-c]pyridin-6-yl)-6-(4-((1-(2-(2,6-dioxopiperidin-3-yl)-1-oxoisoindolin-4-yl)piperidin-4-yl)methoxy)piperidin-1-yl)nicotinamide

To a solution of 6-(4-((1-(2-(2,6-dioxopiperidin-3-yl)-1-oxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)nicotinamide (Example 147, 0.13 g, 0.17 mmol) in dimethylformamide (3 mL) was added N-chlorosuccinimide (0.02 g, 0.18 mmol) and the reaction mixture was stirred at room temperature for 16 h. The reaction mixture was then quenched with ice cold water (10 mL), the resultant precipitate was collected by filtration and dried in vacuo. The crude material was purified by preparative HPLC (Method A) to afford N-(3-chloro-2-((R)-1-methylpyrrolidin-2-yl)-1H-pyrrolo[3,2-c]pyridin-6-yl)-6-(4-((1-(2-(2,6-dioxopiperidin-3-yl)-1-oxoisoindolin-4-yl)piperidin-4-yl)methoxy)piperidin-1-yl)nicotinamide (Example 191, 0.027 g, 20%) as an off-white solid. ¹H NMR (400 MHz, DMSO-d₆) δ ppm 1.34-1.50 (m, 6H), 1.64-1.75 (m, 2H), 1.79-1.99 (m, 9H), 2.15-2.18 (m, 4H), 2.66-2.77 (m, 2H), 2.91-2.93 (m, 1H), 3.15-3.18 (m 1H), 3.37-3.42 (m, 5H), 3.49-3.57 (m, 2H), 3.99-4.03 (m, 2H), 4.26-4.30 (m, 1H), 4.40-4.45 (m, 1H), 5.11 (dd, J=13.33, 5.14 Hz, 1H), 6.90 (d, J=9.17 Hz, 1H), 7.16 (d, J=7.70 Hz, 1H), 7.29 (d, J=7.34 Hz, 1H), 7.40-7.44 (m, 1H), 8.14 (dd, J=9.17, 2.45 Hz, 1H), 8.22 (d, J=0.86 Hz, 1H), 8.48 (s, 1H), 8.80 (d, J=2.45 Hz, 1H), 10.42 (s, 1H), 10.97 (s, 1H), 11.66 (s, 1H). Mass spec m/z 794.5 [M+H]⁺.

### Intermediate 750 was Prepared Following Scheme 185

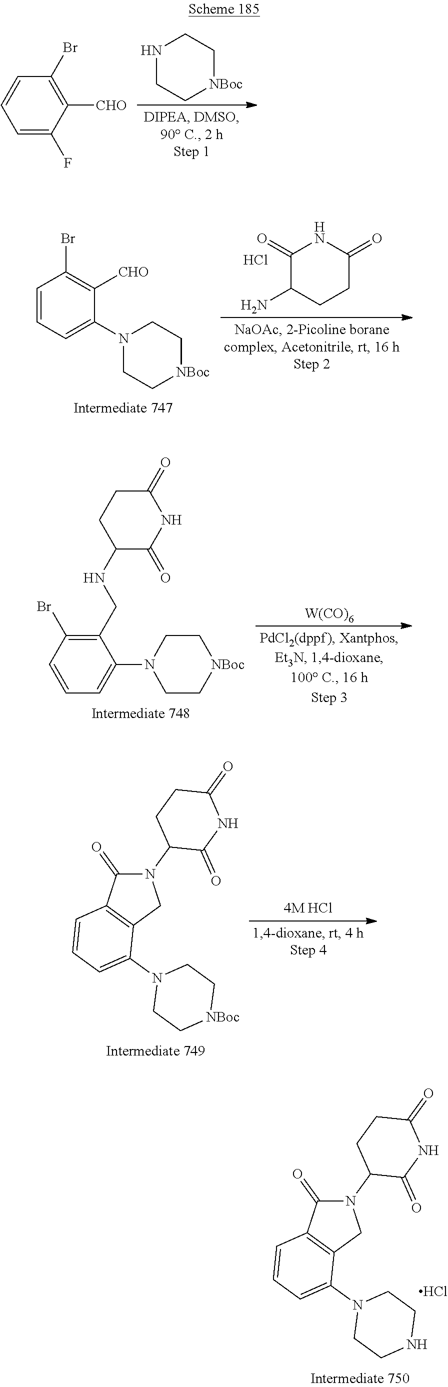

### Step 1

### Intermediate 747: tert-butyl 4-(3-bromo-2-formylphenyl)piperazine-1-carboxylate

To a solution of 2-bromo-6-fluorobenzaldehyde (CAS No. 360575-28-6, 5.0 g, 23.6 mmol) and tert-butyl piperazine-1-carboxylate (CAS No. 57260-71-6, 6.74 g, 23.6 mmol) in dimethyl sulfoxide (50 mL) was added N,N-diisopropylethylamine (12.4 mL, 70.9 mmol) and the reaction mixture was heated at 90° C. for 12 h. The reaction mixture was then quenched with water (50 mL) and extracted with dichloromethane (50 mL). The organic layer was separated, washed with water (50 mL), dried over anhydrous Na₂SO₄ and concentrated in vacuo. The crude material was purified by combi-flash chromatography, by eluting with 50% ethyl acetate in heptane, to afford tert-butyl 4-(3-bromo-2-formylphenyl)piperazine-1-carboxylate (Intermediate 747, 4.2 g, 48%) as a white solid. ¹H NMR (400 MHz, DMSO-d₆) δ ppm 1.41 (s, 9H), 2.95-2.99 (m, 4H), 3.41-3.50 (m, 4H), 7.22-7.25 (m, 1H), 7.37-7.39 (m, 1H), 7.42-7.46 (m, 1H), 10.13 (br s, 1H). Mass spec m/z 370.9 [M+H+2]⁺.

### Step 2

### Intermediate 748: tert-butyl 4-(3-bromo-2-(((2,6-dioxopiperidin-3-yl)amino)methyl)phenyl)piperazine-1-carboxylate

To a solution of tert-butyl 4-(3-bromo-2-formylphenyl)piperazine-1-carboxylate (Intermediate 747, 25.0 g, 67.71 mmol) in acetonitrile (50 mL) was added 3-aminopiperidine-2,6-dione hydrochloride (CAS No. 24666-56-6, 16.7 g, 101.6 mmol) and sodium acetate (8.42 g, 101.6 mmol), followed by borane-2-picoline complex (CAS No. 999-38-0, 15.09 g, 135.4 mmol). The reaction mixture was stirred at room temperature for 16 h. The reaction mixture was then quenched with water (400 mL) and extracted with dichloromethane (600 mL). The organic layer was separated, washed with water (50 mL), dried over anhydrous Na₂SO₄ and concentrated in vacuo. The crude material was purified by combi-flash chromatography, by eluting with 60% ethyl acetate in heptane, to afford tert-butyl 4-(3-bromo-2-(((2,6-dioxopiperidin-3-yl)amino)methyl)phenyl)piperazine-1-carboxylate (Intermediate 748, 12.0 g, 37%) as a white solid. ¹H NMR (400 MHz, DMSO-d₆) δ ppm 1.42 (s, 9H), 2.56-2.58 (m, 2H), 2.75-2.80 (m, 4H), 2.96-3.05 (m, 2H), 3.24-2-3.28 (m, 2H), 3.41-3.50 (m, 3H), 3.89-3.93 (m, 1H), 3.97-4.03 (m, 1H), 7.17-7.25 (m, 2H), 7.34-7.38 (m, 2H), 10.78 (br s, 1H). Mass spec m/z 483.0 [M+H+2]⁺.

### Step 3

### Intermediate 749: tert-butyl 4-(2-(2,6-dioxopiperidin-3-yl)-1-oxoisoindolin-4-yl)piperazine-1-carboxylate

To a solution of tert-butyl 4-(3-bromo-2-(((2,6-dioxopiperidin-3-yl)amino)methyl)phenyl)piperazine-1-carboxylate (Intermediate 748, 12.0 g, 24.9 mmol) in 1,4-dioxane (120 mL) was added triethylamine (10.5 mL, 74.8 mmol). The reaction mixture was purged with argon for 15 min, then Xanthphos (2.97 g, 4.98 mmol), PdCl₂(dppf) (3.84 g, 4.98 mmol) and W(CO)₆ (4.52 g, 12.5 mmol) were added. The reaction mixture was purged with argon for another 10 min and heated at 100° C. for 16 h. The reaction mixture was then diluted with water (1000 mL) and extracted with ethyl acetate (3×500 mL). The combined organic layers were dried over anhydrous Na₂SO₄ and concentrated in vacuo. The crude material was purified by combi-flash chromatography, by eluting with 60% ethyl acetate in heptane, to afford tert-butyl 4-(2-(2,6-dioxopiperidin-3-yl)-1-oxoisoindolin-4-yl)piperazine-1-carboxylate (Intermediate 749, 8.0 g, 75%) as a yellow solid. ¹H NMR (400 MHz, DMSO-d₆) δ ppm 1.42 (s, 9H), 2.42-2.46 (m, 1H), 2.58-2.62 (m, 1H), 2.85-3.03 (m, 5H), 3.16-3.18 (m, 1H), 3.40-3.48 (m, 4H), 4.28-4.33 (m, 1H), 4.44-4.49 (m, 1H), 5.10-5.14 (m, 1H), 7.17-7.22 (m, 1H), 7.33-7.38 (m, 1H), 7.42-7.46 (m, 1H), 10.99 (br s, 1H). Mass spec m/z 427.0 [M−H]⁻.

### Step 4

### Intermediate 750: 3-(1-oxo-4-(piperazin-1-yl)isoindolin-2-yl)piperidine-2,6-dione hydrochloride

To a cooled (0° C.) solution of tert-butyl 4-(2-(2,6-dioxopiperidin-3-yl)-1-oxoisoindolin-4-yl)piperazine-1-carboxylate (Intermediate 749, 1.20 g, 2.80 mmol) in 1,4-dioxane (10 mL) was added 4M hydrochloric acid in 1,4-dioxane (10 mL) and the reaction mixture was stirred at room temperature for 4 h. The reaction mixture was then concentrated in vacuo and the residue triturated with diethyl ether (2 mL) to afford 3-(1-oxo-4-(piperazin-1-yl)isoindolin-2-yl)piperidine-2,6-dione hydrochloride (Intermediate 750, 1.2 g) as an off-white solid, which was used for the next step without further purification. ¹H NMR (400 MHz, DMSO-d₆) δ ppm 1.99-2.02 (m, 1H), 2.45-2.49 (m, 1H), 2.65-2.68 (m, 1H), 2.90-2.98 (m, 1H), 3.22-3.32 (m, 8H), 4.27-4.31 (m, 1H), 4.45-4.50 (m, 1H), 5.12-5.17 (m, 1H), 7.22-7.24 (m, 1H), 7.38-7.40 (m, 1H), 7.48-7.50 (m, 1H), 9.25 (br s, 1H), 11.04 (br s, 2H). Mass spec m/z 329.1 [M+H]⁺.

### Example 192 was Prepared Following Scheme 186

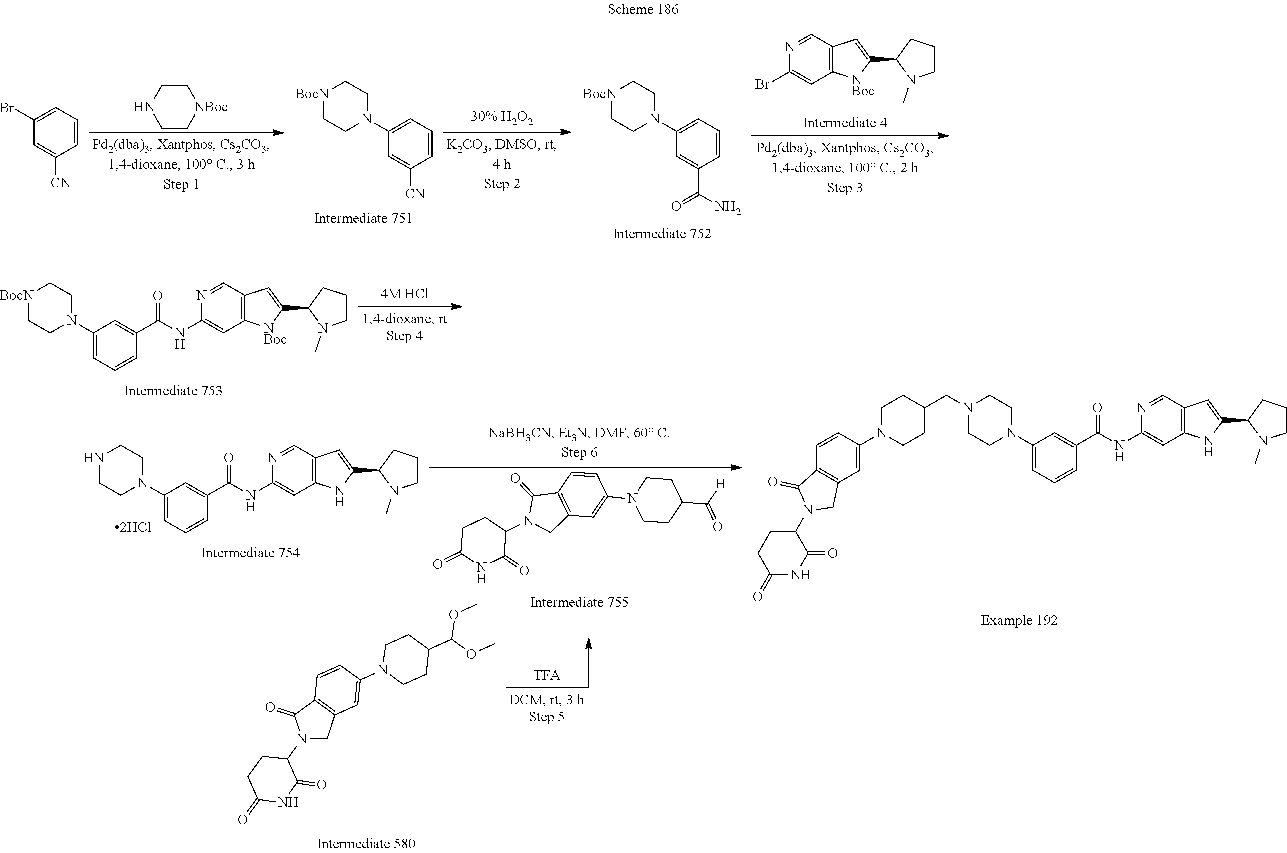

### Step 1

### Intermediate 751: tert-butyl 4-(3-cyanophenyl)piperazine-1-carboxylate

To a solution of 3-bromobenzonitrile (Cas No. 6952-59-6, 2.5 g, 14.0 mmol) in 1,4-dioxane (25 mL) was added tert-butyl piperazine-1-carboxylate (CAS No. 57260-71-6, 2.8 g, 14.0 mmol) followed by cesium carbonate (13.0 g, 41.0 mmol). The reaction mixture was purged with argon for 15 min, then Pd₂(dba)₃ (1.3 g, 1.4 mmol) and Xantphos (1.6 g, 2.7 mmol) were added. The reaction mixture was further purged with argon for 10 min and heated at 100° C. for 3 h. After completion, the reaction mixture was filtered through a celite bed, and the filter pad was washed with ethyl acetate (25 mL). The filtrate was concentrated in vacuo and the crude material was purified by combi-flash chromatography, by eluting with 90% ethyl acetate in heptane, to afford tert-butyl 4-(3-cyanophenyl)piperazine-1-carboxylate (Intermediate 751, 1.5 g, 38%) as a brown solid. ¹H NMR (400 MHz, DMSO-d₆) δ ppm 1.41 (s, 9H), 3.18-3.22 (m, 4H), 3.41-3.47 (m, 4H), 7.18 (d, J=7.46 Hz, 1H), 7.28 (dd, J=8.50, 1.90 Hz, 1H), 7.34 (s, 1H), 7.37-7.42 (m, 1H).

### Step 2

### Intermediate 752: tert-butyl 4-(3-carbamoylphenyl)piperazine-1-carboxylate

To a cooled (0° C.) solution of tert-butyl 4-(3-cyanophenyl)piperazine-1-carboxylate (Intermediate 751, 3.0 g, 10.44 mmol) in dimethyl sulfoxide (30 mL) was added potassium carbonate (4.32 g, 31.32 mmol) and 30% hydrogen peroxide (0.96 mL, 31.32 mmol) and the reaction mixture was stirred at room temperature for 4 h. After completion, the reaction mixture was quenched with ice cold water (200 mL) and the resultant precipitate was collected by filtration and dried in vacuo to afford tert-butyl 4-(3-carbamoylphenyl)piperazine-1-carboxylate (Intermediate 752, 2.5 g, 78%) as a white solid, which was used for the next step without further purification. ¹H NMR (400 MHz, DMSO-d₆) δ ppm 1.42 (s, 9H), 3.10-3.17 (m, 4H), 3.43-3.49 (m, 4H), 7.09 (d, J=7.18 Hz, 1H), 7.29 (d, J=8.31 Hz, 3H), 7.42 (br s, 1H), 7.90 (br s, 1H). Mass spec m/z 306.1 [M+H]⁺.

### Step 3

### Intermediate 753: tert-butyl (R)-6-(3-(4-(tert-butoxycarbonyl)piperazin-1-yl)benzamido)-2-(1-methylpyrrolidin-2-yl)-1H-pyrrolo[3,2-c]pyridine-1-carboxylate

To a solution of tert-butyl 4-(3-carbamoylphenyl)piperazine-1-carboxylate (Intermediate 752, 0.96 g, 3.2 mmol) in 1,4-dioxane (30 mL) was added tert-butyl (R)-6-bromo-2-(1-methylpyrrolidin-2-yl)-1H-pyrrolo[3,2-c]pyridine-1-carboxylate (Intermediate 4, 1.5 g, 3.9 mmol) followed by cesium carbonate (3.9 g, 12.0 mmol). The reaction mixture was purged with argon for 15 min, then Pd₂(dba)₃ (0.56 g, 0.59 mmol) and Xantphos (0.71 g, 1.2 mmol) were added. The reaction mixture was further purged with argon for 10 min and heated at 100° C. for 2 h. After completion, the reaction mixture was filtered through a celite bed and the filter pad was washed with ethyl acetate (10 mL). The filtrate was concentrated in vacuo and the crude material was purified by combi-flash chromatography, by eluting with 90% ethyl acetate in heptane, to afford tert-butyl (R)-6-(3-(4-(tert-butoxycarbonyl)piperazin-1-yl)benzamido)-2-(1-methylpyrrolidin-2-yl)-1H-pyrrolo[3,2-c]pyridine-1-carboxylate (Intermediate 753, 1.2 g, 50%) as a yellow solid. ¹H NMR (400 MHz, DMSO-d₆) δ ppm 1.42 (s, 9H), 1.58-1.62 (m, 2H), 1.69 (s, 9H), 2.30-2.34 (m, 2H), 2.35 (s, 3H), 3.09-3.17 (m, 2H), 3.18-3.25 (m, 4H), 3.45-3.51 (m, 4H), 3.89-3.93 (m, 1H), 6.75 (s, 1H), 7.15 (d, J=8.0 Hz, 1H), 7.35 (t, J=8.0 Hz, 1H), 7.46 (d, J=7.60 Hz, 1H), 7.61 (s, 1H), 8.59 (s, 1H), 8.92 (s, 1H), 10.69 (br s, 1H). Mass spec m/z 605.3 [M+H]⁺.

### Step 4

### Intermediate 754: (R)—N-(2-(1-methylpyrrolidin-2-yl)-1H-pyrrolo[3,2-c]pyridin-6-yl)-3-(piperazin-1-yl)benzamide dihydrochloride

To a cooled (0° C.) solution of tert-butyl (R)-6-(3-(4-(tert-butoxy carbonyl)piperazin-1-yl)benzamido)-2-(1-methylpyrrolidin-2-yl)-1H-pyrrolo[3,2-c]pyridine-1-carboxylate (Intermediate 753, 1.2 g, 2.0 mmol) in 1,4-dioxane (25 mL) was added 4M hydrogen chloride in 1,4-dioxane (25 mL) and the reaction mixture was stirred at room temperature for 4 h. After completion, the reaction mixture was concentrated in vacuo to afford (R)—N-(2-(1-methylpyrrolidin-2-yl)-1H-pyrrolo[3,2-c]pyridin-6-yl)-3-(piperazin-1-yl)benzamide dihydrochloride (Intermediate 754, 1.0 g) as a yellow solid, which was used for the next step without further purification. ¹H NMR (400 MHz, DMSO-d₆) δ ppm 1.26-1.31 (m, 4H), 2.16-2.23 (m, 2H), 2.32-2.42 (m, 2H), 2.54-2.59 (m, 1H), 2.99 (s, 3H), 3.07-3.15 (m, 2H), 3.61-3.74 (m, 2H), 4.76 (d, J=8.31 Hz, 1H), 7.28-7.35 (m, 1H), 7.48 (t, J=7.93 Hz, 1H), 7.64 (d, J=7.55 Hz, 1H), 7.88 (s, 1H), 8.41 (s, 1H), 9.13 (s, 1H), 9.41-9.46 (m, 2H), 11.60 (br s, 1H), 12.24 (br s, 1H), 13.39 (br s, 1H).

### Step 5

### Intermediate 755: 1-(2-(2,6-dioxopiperidin-3-yl)-1-oxoisoindolin-5-yl)piperidine-4-carbaldehyde

To a solution of 3-(5-(4-(dimethoxymethyl)piperidin-1-yl)-1-oxoisoindolin-2-yl)piperidine-2,6-dione (Intermediate 580, 1.5 g, 3.7 mmol) in dichloromethane (20 mL) was added trifluoroacetic acid (18 mL) and the mixture was stirred at room temperature for 5 h. After completion, the reaction mixture was concentrated in vacuo to afford 1-(2-(2,6-dioxopiperidin-3-yl)-1-oxoisoindolin-5-yl)piperidine-4-carbaldehyde (Intermediate 755, 1.0 g, 75%) as a yellow solid, which was used for the next step without further purification. ¹H NMR (400 MHz, DMSO-d₆) δ ppm 1.64-1.70 (m, 2H), 1.94-1.99 (m, 4H), 2.56-2.68 (m, 2H), 2.82-2.91 (m, 3H), 3.30-3.38 (m, 2H), 4.27-4.32 (m, 1H), 4.42-4.48 (m, 1H), 5.09-5.14 (m, 1H), 7.19 (d, J=7.83 Hz, 2H), 7.57 (d, J=7.04 Hz, 1H), 9.67 (br s, 1H), 10.98 (br s, 1H). Mass spec m/z 356.1 [M+H]⁺.

### Step 6

### Example 192: 3-(4-((1-(2-(2,6-dioxopiperidin-3-yl)-1-oxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide

To a cooled (0° C.) solution of (R)—N-(2-(1-methylpyrrolidin-2-yl)-1H-pyrrolo[3,2-c]pyridin-6-yl)-3-(piperazin-1-yl)benzamide hydrochloride (Intermediate 754, 0.81 g, 2.02 mmol) in dimethylformamide (15 mL) was added 1-(2-(2,6-dioxopiperidin-3-yl)-1-oxoisoindolin-5-yl)piperidine-4-carbaldehyde (Intermediate 755, 0.60 g, 1.68 mmol) and sodium cyanoborohydride (0.24 g, 3.37 mmol) and the reaction mixture was heated at 60° C. for 5 h. After completion, the reaction mixture was quenched with water (50 mL) and extracted with ethyl acetate (2×60 mL). The organic layer was separated, dried over anhydrous Na₂SO₄ and concentrated in vacuo. The crude material was purified by preparative HPLC (Method A) to afford 3-(4-((1-(2-(2,6-dioxopiperidin-3-yl)-1-oxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide (Example 192, 0.10 g, 8%) as an off-white solid. ¹H NMR (400 MHz, DMSO-d₆) δ ppm 1.16-1.28 (m, 2H), 1.81-1.99 (m, 7H), 2.17 (s, 3H), 2.21-2.38 (m, 5H), 2.53-2.57 (m, 4H), 2.80-2.93 (m, 3H), 3.12-3.18 (m, 1H), 3.23-3.27 (m, 4H), 3.31-3.32 (m, 2H), 3.87-3.91 (m, 2H), 4.15-4.23 (m, 1H), 4.27-4.36 (m, 1H), 5.01-5.06 (m, 1H), 6.39 (s, 1H), 7.03-7.08 (m, 2H), 7.12 (dd, J=8.25, 1.63 Hz, 1H), 7.32 (t, J=7.94 Hz, 1H), 7.44 (d, J=7.63 Hz, 1H), 7.50 (d, J=8.88 Hz, 1H), 7.60 (s, 1H), 8.16 (s, 1H), 8.18 (s, 1H), 10.43 (br s, 1H), 10.93 (br s, 1H), 11.36 (br s, 1H). Mass spec m/z 744.0 [M+H]⁺.

### Example 193 was Prepared Following Scheme 187

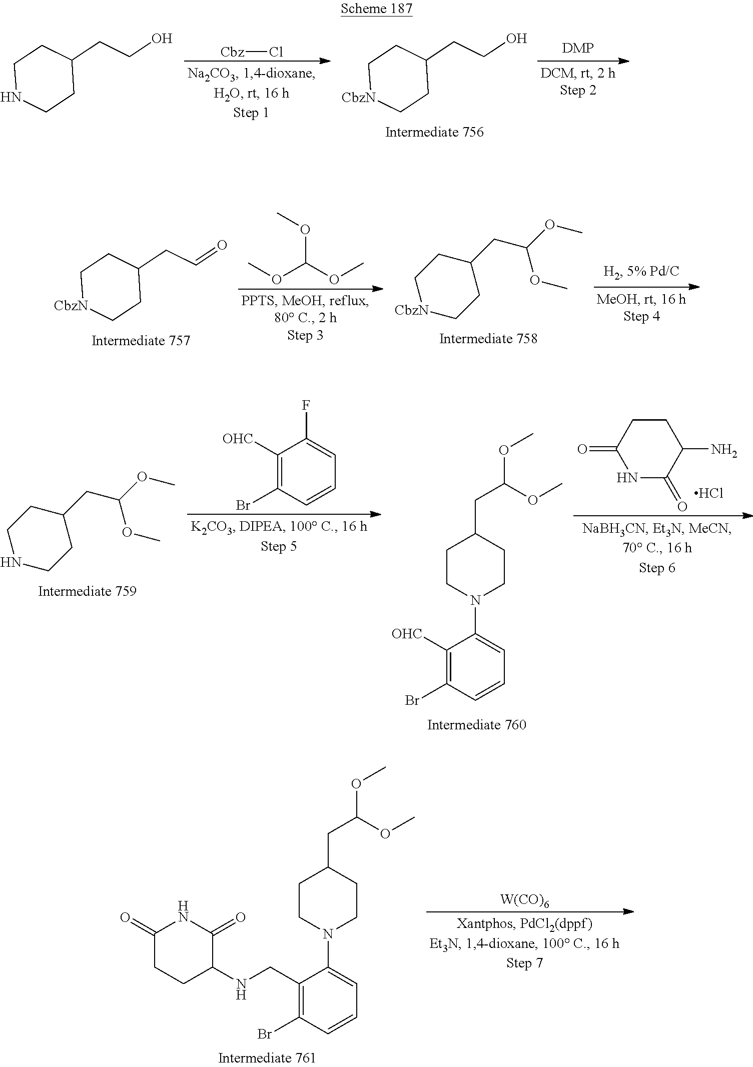

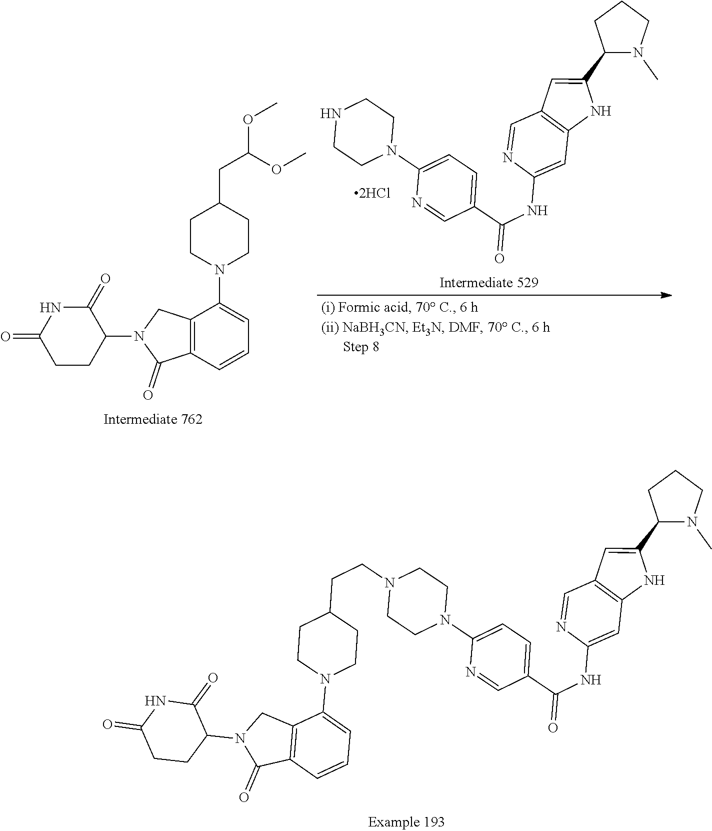

### Step 1

### Intermediate 756: benzyl 4-(2-hydroxyethyl)piperidine-1-carboxylate

To a cooled (0° C.) solution of 2-(piperidin-4-yl)ethan-1-ol (CAS No. 622-26-4, 24.0 g, 186 mmol) in 1,4-dioxane (360 mL) and water (360 mL) was added sodium carbonate (39.77 g, 372 mmol) and benzyl chloroformate (3M in toluene, 69.71 g, 204 mmol). The reaction mixture was stirred at room temperature for 16 h, then diluted with water (100 mL) and extracted with ethyl acetate (3×500 mL). The combined organic layers were dried over anhydrous Na₂SO₄, filtered, and concentrated in vacuo to afford benzyl 4-(2-hydroxyethyl)piperidine-1-carboxylate (Intermediate 756, 50.0 g, 94%) as a white solid. ¹H NMR (400 MHz, DMSO-d₆) δ ppm 0.91-1.05 (m, 2H), 1.31-1.36 (m, 2H), 1.53-1.64 (m, 3H), 2.73-2.80 (m, 2H), 3.40-3.46 (m, 2H), 3.94-4.30 (m, 2H), 4.34-4.38 (m, 1H), 5.05 (s, 2H), 7.28-7.40 (m, 5H). Mass spec m/z 264.2 [M+H]⁺.

### Step 2

### Intermediate 757: benzyl 4-(2-oxoethyl)piperidine-1-carboxylate

To a cooled (0° C.) solution of benzyl 4-(2-hydroxyethyl)piperidine-1-carboxylate (Intermediate 756, 25.0 g, 95.0 mmol) in dichloromethane (250 mL) was added Dess-martin periodinane (84.8 g, 190 mmol) and the reaction mixture was stirred at room temperature for 2 h. The reaction mixture was then quenched with saturated aqueous sodium bicarbonate solution (300 mL) and extracted with dichloromethane (3×300 mL). The combined organic layers were dried over anhydrous Na₂SO₄, filtered, and concentrated in vacuo. The crude material was purified by combi-flash chromatography, by eluting with 15% ethyl acetate in heptane, to afford benzyl 4-(2-oxoethyl)piperidine-1-carboxylate (Intermediate 757, 14.0 g, 56%) as a colourless liquid. ¹H NMR (400 MHz, DMSO-d₆) δ ppm 1.02-1.12 (m, 2H), 1.60-1.65 (m, 2H), 1.90-2.03 (m, 1H), 2.35-2.38 (m, 2H), 2.71-2.90 (m, 2H), 3.94-3.98 (m, 2H), 5.06 (s, 2H), 7.30-7.37 (m, 5H), 9.66 (s, 1H). Mass spec m/z 262.2 [M+H]⁺.

### Step 3

### Intermediate 758: benzyl 4-(2,2-dimethoxyethyl)piperidine-1-carboxylate

To a solution of benzyl 4-(2-oxoethyl)piperidine-1-carboxylate (Intermediate 757, 27.0 g, 103 mmol) in methanol (270 mL) was added pyridinium p-toluenesulfonate (5.29 g, 20.7 mmol) and trimethyl orthoformate (CAS No. 149-73-5, 11.0 g, 103 mmol). The reaction mixture was heated at 80° C. for 2 h. The reaction mixture was concentrated in vacuo and the crude material purified by combi-flash chromatography, by eluting with 14% ethyl acetate in heptane, to afford benzyl 4-(2,2-dimethoxyethyl)piperidine-1-carboxylate (Intermediate 758, 20.0 g, 63%) as colorless liquid. ¹H NMR (400 MHz, DMSO-d₆) δ ppm 1.01-1.09 (m, 2H), 1.43-1.54 (m, 3H), 1.62-1.68 (m, 2H), 2.65-2.84 (m, 2H), 3.21 (s, 6H), 3.94-4.00 (m, 2H), 4.44 (s, 1H), 5.06 (s, 2H), 7.23-7.40 (m, 5H).

### Step 4

### Intermediate 759: 4-(2,2-dimethoxyethyl)piperidine

To a solution of benzyl 4-(2,2-dimethoxyethyl)piperidine-1-carboxylate (Intermediate 758, 20.0 g, 65.1 mmol) in methanol (200 mL) was added 5% Pd/C (20.0 g, 188 mmol) and the reaction mixture was stirred at room temperature for 16 h under a 150 psi H₂ atmosphere. After completion, the reaction mixture was filtered through a celite bed and the filtrate concentrated in vacuo to afford 4-(2,2-dimethoxyethyl)piperidine (Intermediate 759, 11.0 g, 98%) as colorless liquid, which was used for the next step without further purification. ¹H NMR (400 MHz, DMSO-d₆) δ ppm 1.17-1.26 (m, 2H), 1.42-1.58 (m, 3H), 1.68-1.71 (m, 2H), 2.61-2.67 (m, 2H), 3.05-3.08 (m, 2H), 3.16-3.22 (s, 7H), 4.41-4.46 (s, 1H).

### Step 5

### Intermediate 760: 2-bromo-6-(4-(2,2-dimethoxyethyl)piperidin-1-yl)benzaldehyde

To a solution of 4-(2,2-dimethoxyethyl)piperidine (Intermediate 759, 5.0 g, 28.9 mmol) and 2-bromo-6-fluoro-benzaldehyde (CAS No. 360575-28-6, 6.44 g, 31.7 mmol) in dimethyl sulfoxide (30 mL) was added N,N-diisopropylethylamine (20.2 mL, 115 mmol) and the reaction mixture was heated at 100° C. for 16 h. The reaction mixture was then quenched with ice cold water (200 mL), the resultant precipitate was collected by filtration and dried in vacuo to afford 2-bromo-6-(4-(2,2-dimethoxyethyl)piperidin-1-yl)benzaldehyde (Intermediate 760, 6.0 g, 45%) as an off-white solid, which was used for the next step without further purification. Mass spec m/z 357.7 [M+H+2]⁺.

### Step 6

### Intermediate 761: 3-((2-bromo-6-(4-(2,2-dimethoxyethyl)piperidin-1-yl)benzyl)amino)piperidine-2,6-dione

To a solution of 2-bromo-6-(4-(2,2-dimethoxyethyl)piperidin-1-yl)benzaldehyde (Intermediate 760, 8.60 g, 24.0 mmol) and 3-aminopiperidine-2,6-dione hydrochloride (CAS No. 24666-56-6, 4.4 g, 27.0 mmol) in acetonitrile (45 mL) was added triethylamine (14 mL, 97.0 mmol) and sodium cyanoborohydride (4.80 g, 72.0 mmol). The reaction mixture was heated at 70° C. for 16 h. After completion, the reaction mixture was filtered through filter paper and the filtrate concentrated in vacuo. The crude material was purified by combi-flash chromatography, by eluting with 50% ethyl acetate in heptane, to afford 3-((2-bromo-6-(4-(2,2-dimethoxyethyl)piperidin-1-yl)benzyl)amino)piperidine-2,6-dione (Intermediate 761, 10.0 g, 84%) as a white solid. ¹H NMR (400 MHz, DMSO-d₆) δ ppm 1.28-1.54 (m, 6H), 1.70-1.83 (m, 3H), 2.26-2.33 (m, 1H), 2.51-2.58 (m, 2H), 2.70-2.78 (m, 1H), 2.90-3.00 (m, 1H), 3.02-3.08 (m, 1H), 3.20-3.28 (s, 8H), 3.86-3.97 (m, 2H), 4.46-4.51 (m, 1H), 7.13-7.20 (m, 2H), 7.31-7.34 (m, 1H), 10.77 (s, 1H). Mass spec m/z 468.0 [M+H]⁺.

### Step 7

### Intermediate 762: 3-(4-(4-(2,2-dimethoxyethyl)piperidin-1-yl)-1-oxoisoindolin-2-yl)piperidine-2,6-dione

To a solution of 3-((2-bromo-6-(4-(2,2-dimethoxyethyl)piperidin-1-yl)benzyl)amino)piperidine-2,6-dione (Intermediate 761, 3.0 g, 6.60 mmol) in 1,4-dioxane (45 mL) was added triethylamine (2.77 mL, 19.81 mmol). The reaction mixture was purged with argon for 15 min then Pd(dppf)Cl₂ (1.01 g, 1.32 mmol) followed by Xantphos (0.78 g, 1.32 mmol) and W(CO)₆ (1.19 g, 3.01 mmol) were added. The reaction mixture was further purged with argon for 10 min and heated at 100° C. for 16 h. The reaction mixture was filtered through a celite bed, the filter pad was washed with ethyl acetate (100 mL) and the filtrate was concentrated in vacuo. The crude material was purified by combi-flash chromatography, by eluting with 30% ethyl acetate in heptane, to afford 3-(4-(4-(2,2-dimethoxyethyl)piperidin-1-yl)-1-oxoisoindolin-2-yl)piperidine-2,6-dione (Intermediate 762, 0.80 g, 29%) as a white solid. ¹H NMR (400 MHz, DMSO-d₆) δ ppm 1.28-1.48 (m, 2H), 1.49-1.52 (m, 3H), 1.74-1.81 (m, 2H), 1.94-2.02 (m, 1H), 2.47-2.49 (m, 1H), 2.53-2.60 (m, 1H), 2.67-2.73 (m, 2H), 2.86-2.92 (m, 1H), 3.22 (s, 6H), 3.32-3.38 (m, 2H), 4.24-4.31 (m, 1H), 4.39-4.50 (m, 2H), 5.07-5.15 (m, 1H), 7.15 (d, J=8.00 Hz, 1H), 7.29 (d, J=7.60 Hz, 1H), 7.42 (d, J=7.60 Hz, 1H), 10.97 (s, 1H). Mass spec m/z 416.1 [M+H]⁺.

### Step 8

### Example 193: 6-(4-(2-(1-(2-(2,6-dioxopiperidin-3-yl)-1-oxoisoindolin-4-yl)piperidin-4-yl)ethyl)piperazin-1-yl)-N-(2-((R)-1-methylpyrrolidin-2-yl)-1H-pyrrolo[3,2-c]pyridin-6-yl)nicotinamide

A solution of 3-(4-(4-(2,2-dimethoxyethyl)piperidin-1-yl)-1-oxoisoindolin-2-yl)piperidine-2,6-dione (Intermediate 762, 0.700 g, 1.68 mmol) in formic acid (7 mL) was stirred at 0° C. for 2 h. The reaction mixture was concentrated in vacuo. A solution of (R)—N-(2-(1-methylpyrrolidin-2-yl)-1H-pyrrolo[3,2-c]pyridin-6-yl)-6-(piperazin-1-yl)nicotinamide dihydrochloride (Intermediate 529, 0.60 g, 1.23 mmol) in dimethylformamide (10 mL) and triethylamine (0.63 mL, 4.94 mmol) were then added and the reaction mixture was heated at 70° C. for 6 h. Sodium cyanoborohydride (0.24 g, 3.71 mmol) was added and the reaction mixture was heated at 70° C. for 6 h. The mixture was filtered through a celite bed and the filter pad was washed with ethyl acetate (200 mL). The filtrate was concentrated in vacuo and the crude material was purified by preparative HPLC (Method A) to afford 6-(4-(2-(1-(2-(2,6-dioxopiperidin-3-yl)-1-oxoisoindolin-4-yl)piperidin-4-yl)ethyl)piperazin-1-yl)-N-(2-((R)-1-methylpyrrolidin-2-yl)-1H-pyrrolo[3,2-c]pyridin-6-yl)nicotinamide (Example 193, 0.16 g, 17%) as brown solid. ¹H NMR (400 MHz, DMSO-d₆) δ ppm 1.28-1.39 (m, 2H), 1.46-1.51 (m, 3H), 1.74-2.03 (m, 6H), 2.13-2.18 (m, 4H), 2.23-2.30 (m, 1H), 2.36-2.40 (m, 2H), 2.41-2.49 (m, 4H), 2.52-2.61 (m, 1H), 2.73-2.78 (m, 2H), 2.84-2.95 (m, 1H), 3.12-3.17 (m, 1H), 3.27-3.42 (m, 4H), 3.61-3.64 (m, 4H), 4.26-4.31 (m, 1H), 4.40-4.45 (m, 1H), 5.09-5.13 (m, 1H), 6.38 (s, 1H), 6.86 (d, J=8.00 Hz, 1H), 7.16 (d, J=7.60 Hz, 1H), 7.29 (d, J=7.20 Hz, 1H), 7.42 (d, J=7.60 Hz, 1H), 8.15-8.18 (m, 2H), 8.48 (s, 1H), 8,79 (s, 1H), 10.28 (s, 1H), 11.00 (s, 1H), 11.31 (s, 1H). Mass spec m/z 759.3 [M+H]⁺.

### Example 194 was Prepared Following Scheme 188

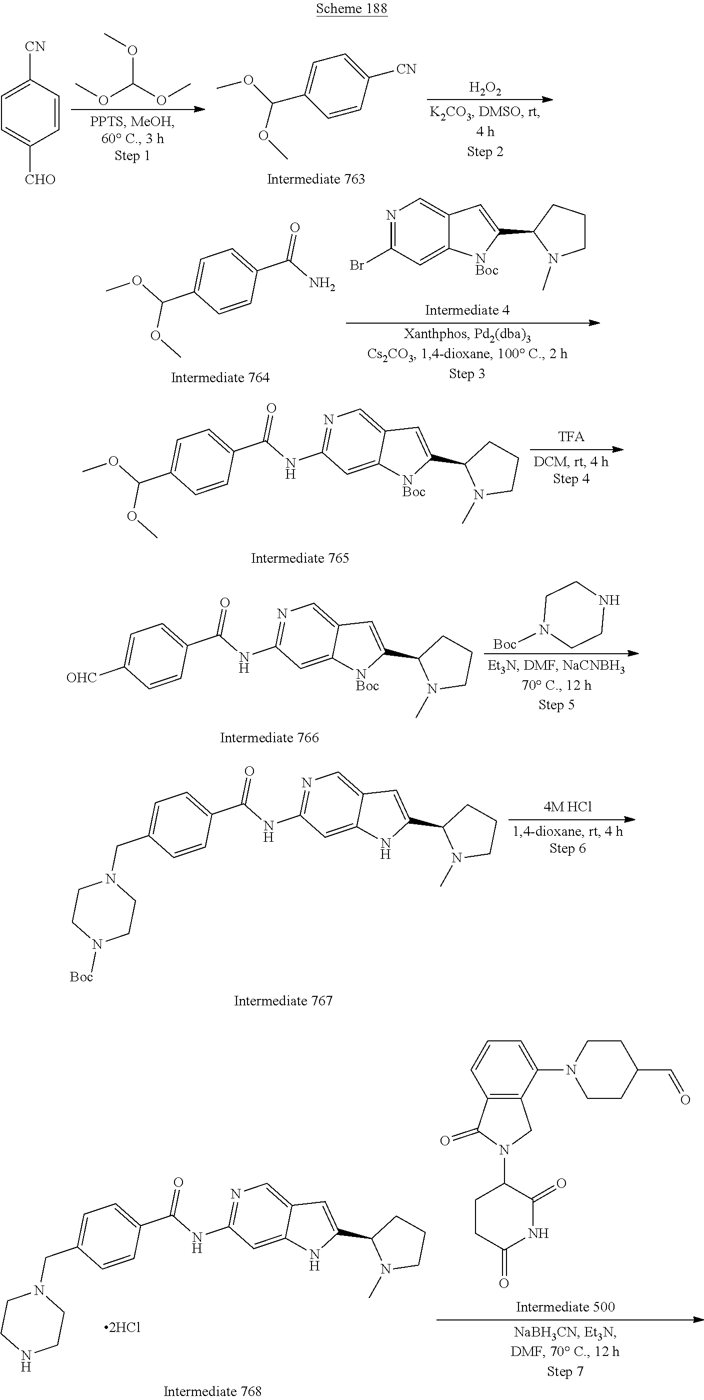

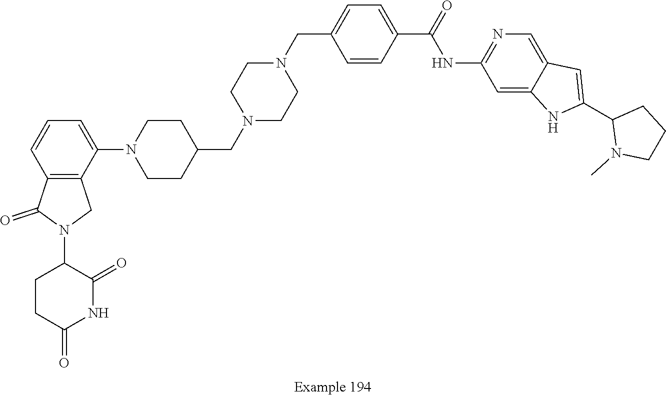 [SMILES: CN1CCCC1c1cc2cnc(NC(=O)c3ccc(CN4CCN(CC5CCN(c6cccc7c6CN(C6CCC(=O)NC6=O)C7=O)CC5)CC4)cc3)cc2[nH]1]

### Step 1

### Intermediate 763: 4-(dimethoxymethyl)benzonitrile

To a solution of 4-formylbenzonitrile (CAS No. 105-07-7, 10.0 g, 76.3 mmol) in methanol (150 mL) was added trimethyl orthoformate (83.0 mL, 763 mmol) and pyridinium p-toluenesulfonate (3.91 g, 15.3 mmol) and the reaction mixture was heated at 60° C. for 3 h. The reaction mixture was then concentrated in vacuo and the crude material purified by combi-flash chromatography, by eluting with 30% ethyl acetate in heptane, to afford 4-(dimethoxymethyl)benzonitrile (Intermediate 763, 7.5 g, 56%) as a colourless liquid. ¹H NMR (400 MHz, DMSO-d₆) δ ppm 3.27 (s, 6H), 5.45 (s, 1H), 7.58 (d, J=7.60 Hz, 2H), 7.87 (d, J=7.20 Hz, 2H).

### Step 2

### Intermediate 764: 4-(dimethoxymethyl)benzamide

To a cooled (0° C.) solution of 4-(dimethoxymethyl)benzonitrile (Intermediate 763, 10.0 g, 56.4 mmol) in dimethyl sulfoxide (100 mL) was added potassium carbonate (15.6 g, 113 mmol) followed by 30% hydrogen peroxide (5.19 mL, 169 mmol) and the reaction mixture was stirred at room temperature for 4 h. The reaction mixture was quenched with ice cold water (200 mL), the resultant precipitate was collected by filtration and dried in vacuo to afford 4-(dimethoxymethyl)benzamide (Intermediate 764, 11.0 g, 99%) as a white solid, which was used for next step without further purification. ¹H NMR (400 MHz, DMSO-d₆) δ ppm 3.24 (s, 6H), 5.44 (s, 1H), 7.39 (s, 1H), 7.44 (d, J=8.40 Hz, 2H), 7.88 (d, J=8.00 Hz, 2H), 8.00 (s, 1H). Mass spec m/z 196.2 [M+H]⁺.

### Step 3

### Intermediate 765: tert-butyl (R)-6-(4-(dimethoxymethyl)benzamido)-2-(1-methylpyrrolidin-2-yl)-1H-pyrrolo[3,2-c]pyridine-1-carboxylate

To a solution of tert-butyl (R)-6-bromo-2-(1-methylpyrrolidin-2-yl)-1H-pyrrolo[3,2-c]pyridine-1-carboxylate (Intermediate 4, 2.0 g, 5.25 mmol) and 4-(dimethoxymethyl)benzamide (Intermediate 764, 0.82 g, 4.20 mmol) in 1,4-dioxane (20 mL) was added cesium carbonate (5.15 g, 15.8 mmol). The reaction mixture was purged with argon for 15 min, then Xantphos (0.47 g, 0.78 mmol) followed by Pd₂(dba)₃ (1.48 g, 1.57 mmol) were added. The reaction mixture was further purged with argon for 10 min and heated at 100° C. for 2 h. The reaction mixture was filtered through a celite bed and the filter padinu was washed with ethyl acetate (100 mL). The filtrate was concentrated in vacuo and the crude material was purified by combi-flash chromatography, by eluting with 100% ethyl acetate, to afford tert-butyl (R)-6-(4-(dimethoxymethyl)benzamido)-2-(1-methylpyrrolidin-2-yl)-1H-pyrrolo[3,2-c]pyridine-1-carboxylate (Intermediate 765, 1.30 g, 50%) as a white solid. ¹H NMR (400 MHz, DMSO-d₆) δ ppm 1.58-1.79 (m, 13H), 2.31-2.41 (m, 5H), 3.28 (s, 6H), 3.98-3.95 (m, 1H), 5.47 (s, 1H), 6.75 (s, 1H), 7.50 (d, J=8.40 Hz, 2H), 8.05 (d, J=8.00 Hz, 2H), 8.60 (s, 1H), 8.93 (s, 1H), 10.75 (s, 1H). Mass spec m/z 495.5 [M+H]⁺.

### Step 4

### Intermediate 766: (R)-4-formyl-N-(2-(1-methylpyrrolidin-2-yl)-1H-pyrrolo[3,2-c]pyridin-6-yl)benzamide

To a cooled (0° C.) solution of tert-butyl (R)-6-(4-(dimethoxymethyl)benzamido)-2-(1-methylpyrrolidin-2-yl)-1H-pyrrolo[3,2-c]pyridine-1-carboxylate (Intermediate 765, 0.50 g, 1.01 mmol) in dichloromethane (5 mL) was added trifluoroacetic acid (1.16 mL, 15.2 mmol) and the reaction mixture was stirred at room temperature for 4 h. The reaction mixture was then concentrated in vacuo to afford (R)-4-formyl-N-(2-(1-methylpyrrolidin-2-yl)-1H-pyrrolo[3,2-c]pyridin-6-yl)benzamide (Intermediate 766, 0.40 g, 88%) as an off-white solid, which was used for next step without further purification. ¹H NMR (400 MHz, DMSO-d₆) δ ppm 1.01-1.09 (m, 2H), 2.17-2.22 (m, 2H), 2.60-2.65 (m, 2H), 2.85 (s, 3H), 3.71-3.75 (m, 1H), 7.14 (s, 1H), 7.31-7.35 (m, 1H), 7.47-7.50 (m, 1H), 8.05-8.11 (m, 2H), 8.21-8.24 (m, 1H), 9.01 (s, 1H), 10.13 (br s, 1H), 11.71 (br s, 1H), 12.88 (br s, 1H).

### Step 5

### Intermediate 767: tert-butyl (R)-4-(4-((2-(1-methylpyrrolidin-2-yl)-1H-pyrrolo[3,2-c]pyridin-6-yl)carbamoyl)benzyl)piperazine-1-carboxylate

To a solution of (R)-4-formyl-N-(2-(1-methylpyrrolidin-2-yl)-1H-pyrrolo[3,2-c]pyridin-6-yl)benzamide (Intermediate 766, 0.60 g, 1.72 mmol) and tert-butyl piperazine-1-carboxylate (CAS No. 57260-71-6, 0.38 g, 2.06 mmol) in dimethylformamide (6 mL) was added triethylamine (0.52 g, 5.16 mmol) and the reaction mixture was heated at 70° C. for 2 h. Sodium cyanoborohydride (0.34 g, 5.16 mmol) was then added and the reaction mixture was heated at 70° C. for 12 h. The reaction mixture was then quenched with the ice-cold water (100 mL), the resultant precipitate was collected by filtration and dried in vacuo to afford tert-butyl (R)-4-(4-((2-(1-methylpyrrolidin-2-yl)-1H-pyrrolo[3,2-c]pyridin-6-yl)carbamoyl)benzyl)piperazine-1-carboxylate (Intermediate 767, 0.30 g, 32%) as an off-white solid, which was used for next step without further purification. ¹H NMR (400 MHz, DMSO-d₆) δ ppm 1.38 (s, 9H), 1.76-1.89 (m, 4H), 2.13-2.18 (m, 4H), 2.31-2.37 (m 3H), 2.82-2.89 (m, 6H), 3.12-3.18 (m, 3H), 7.42 (d, J=8.40 Hz, 2H), 7.95 (s, 1H), 8.01 (d, J=8.00 Hz, 2H), 8.19 (s, 1H), 8.50 (s, 1H), 10.43 (s, 1H), 11.29 (s, 1H). Mass spec m/z 519.3 [M+H]⁺.

### Step 6

### Intermediate 768: (R)—N-(2-(1-methylpyrrolidin-2-yl)-1H-pyrrolo[3,2-c]pyridin-6-yl)-4-(piperazin-1-ylmethyl)benzamide hydrochloride

To a cooled (0° C.) solution of tert-butyl (R)-4-(4-((2-(1-methylpyrrolidin-2-yl)-1H-pyrrolo[3,2-c]pyridin-6-yl)carbamoyl)benzyl)piperazine-1-carboxylate (Intermediate 767, 0.30 g, 0.57 mmol) in 1,4-dioxane (2 mL) was added 4M hydrochloric acid in 1,4-dioxane (1.5 mL) and the reaction mixture was stirred at room temperature for 4 h. The reaction mixture was then concentrated in vacuo to afford (R)—N-(2-(1-methylpyrrolidin-2-yl)-1H-pyrrolo[3,2-c]pyridin-6-yl)-4-(piperazin-1-ylmethyl)benzamide hydrochloride (Intermediate 768, 0.28 g) as a white solid, which was used for the next step without further purification. Mass spec m/z 419.2 [M+H]⁺.

### Step 7

### Example 194: 4-((4-((1-(2-(2,6-dioxopiperidin-3-yl)-1-oxoisoindolin-4-yl)piperidin-4-yl)methyl)piperazin-1-yl)methyl)-N-(2-((R)-1-methylpyrrolidin-2-yl)-1H-pyrrolo[3,2-c]pyridin-6-yl)benzamide

To a solution of tert-butyl (R)-4-(4-((2-(1-methylpyrrolidin-2-yl)-1H-pyrrolo[3,2-c]pyridin-6-yl)carbamoyl)benzyl)piperazine-1-carboxylate (Intermediate 768, 0.56 g, 1.35 mmol) and 1-(2-(2,6-dioxopiperidin-3-yl)-1-oxoisoindolin-4-yl)piperidine-4-carbaldehyde (Intermediate 500, 0.40 g, 1.12 mmol) in dimethylformamide (5 mL) was added triethylamine (0.47 mL, 3.37 mmol) and the reaction mixture was heated at 70° C. for 2 h. Sodium cyanoborohydride (0.22 g, 3.37 mmol) was then added at room temperature and the reaction mixture was heated at 70° C. for 12 h. The reaction mixture was then poured into ice cold water (30 mL), the resultant precipitate was collected by filtration and dried in vacuo. The crude material was purified by preparative HPLC (Method A) to afford 4-((4-((1-(2-(2,6-dioxopiperidin-3-yl)-1-oxoisoindolin-4-yl)piperidin-4-yl)methyl)piperazin-1-yl)methyl)-N-(2-((R)-1-methylpyrrolidin-2-yl)-1H-pyrrolo[3,2-c]pyridin-6-yl)benzamide (Example 194, 0.01 g, 2%) as an off-white solid. ¹H NMR (400 MHz, DMSO-d₆) δ ppm 1.21-1.30 (m, 2H), 1.63-2.01 (m, 8H), 2.12-2.20 (m, 6H), 2.22-2.31 (m 2H), 2.38-2.45 (m, 6H), 2.52-2.61 (m, 3H), 2.67-2.73 (m, 2H), 2.87-2.94 (m, 2H), 3.12-3.18 (m, 2H), 3.41-3.49 (m, 2H), 4.25-4.29 (m, 1H), 4.39-4.44 (m, 1H), 5.08-5.13 (m, 1H), 6.39 (s, 1H), 7.14 (d, J=8.00 Hz, 1H), 7.28 (d, J=7.20 Hz, 1H), 7.40-7.44 (m, 3H), 8.01 (d, J=8.00 Hz, 2H), 8.19 (s, 1H), 8.50 (s, 1H), 10.40 (s, 1H), 10.98 (s, 1H), 11.37 (s, 1H). Mass spec m/z 758.0 [M+H]⁺.

### Example 195 was Prepared Following Scheme 189

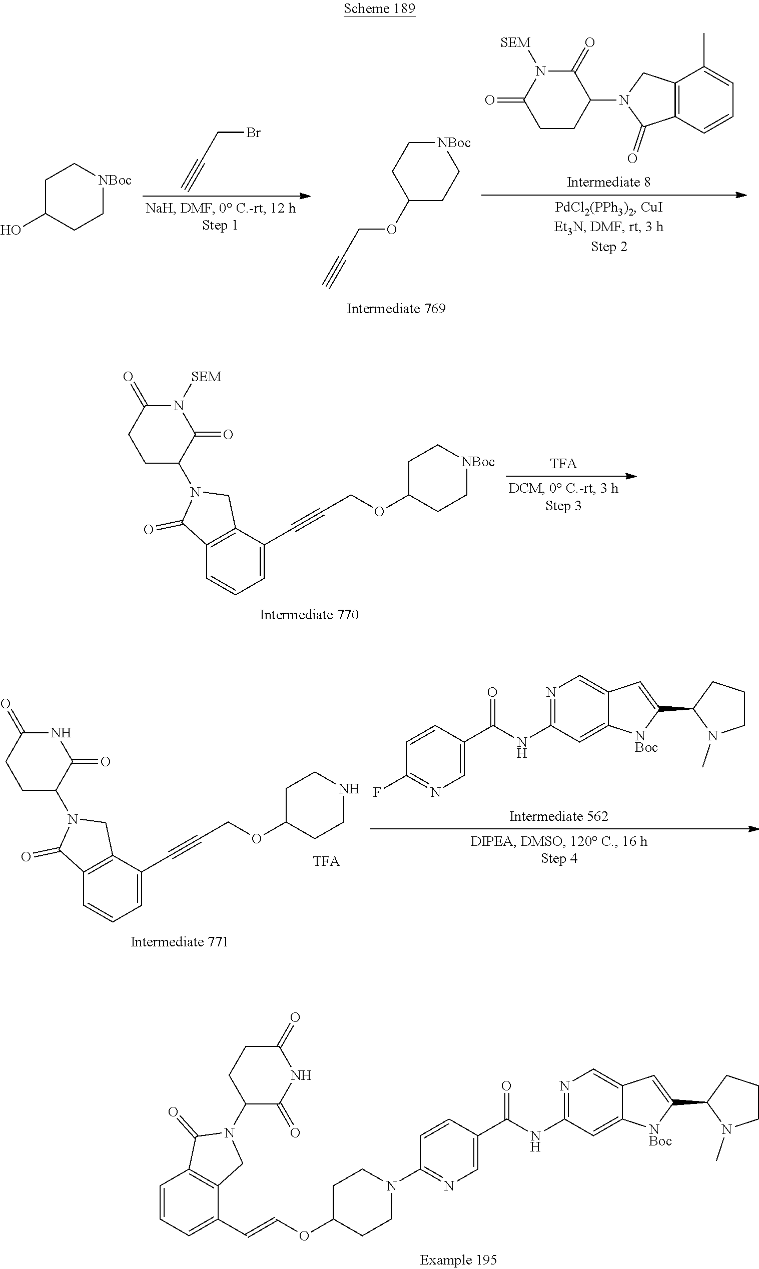

### Step 1

### Intermediate 769: tert-butyl 4-(prop-2-yn-1-yloxy)piperidine-1-carboxylate

To a cooled (0° C.) solution of tert-butyl 4-hydroxypiperidine-1-carboxylate (CAS No. 109384-19-2, 5.0 g, 24.9 mmol) in dimethylformamide (15 mL) was added sodium hydride (60% in mineral oil, 1.49 g, 37.4 mmol) at 0° C. and the reaction was stirred at 0° C. for 30 min. 3-Bromo-prop-1-yne (CAS No. 106-96-7, 3.69 g, 31.1 mmol) was then added and the reaction mixture was stirred at room temperature for 12 h. The reaction mixture was then quenched with ice cold water (100 mL), the resultant precipitate was collected by filtration and dried in vacuo. The crude material was purified by combi-flash chromatography, by eluting with 40% ethyl acetate in heptane, to afford tert-butyl 4-prop-2-ynoxypiperidine-1-carboxylate (Intermediate 769, 4.5 g, 76%) as an off-white solid. ¹H NMR (400 MHz, DMSO-d₆) δ ppm 1.28-1.34 (m, 2H), 1.38 (s, 9H), 1.40-1.44 (m, 1H), 1.72-1.85 (m, 2H), 2.96-3.08 (m, 2H), 3.54-3.62 (m, 3H), 4.16 (s, 2H).

### Step 2

### Intermediate 770: tert-butyl 4-((3-(2-(2,6-dioxo-1-((2-(trimethylsilyl)ethoxy)methyl)piperidin-3-yl)-1-oxoisoindolin-4-yl)prop-2-yn-1-yl)oxy)piperidine-1-carboxylate

To a solution of tert-butyl 4-(prop-2-yn-1-yloxy)piperidine-1-carboxylate (Intermediate 8, 5.02 g, 21.0 mmol) and 3-(4-iodo-1-oxo-isoindolin-2-yl)-1-(2-trimethylsilylethoxymethyl)piperidine-2,6-dione (Intermediate 769, 3.5 g, 6.99 mmol) in tetrahydrofuran (50 mL) was added triethylamine (6.82 mL, 49.0 mmol) and the reaction mixture was purged with argon for 15 min. PdCl₂(PPh₃)₂ (0.49 g, 0.69 mmol) and copper(I) iodide (0.66 g, 3.5 mmol) were then added. The reaction mixture was further purged with argon for 10 min and stirred at room temperature 3 h. After completion, the reaction mixture was filtered through filter paper and the filtrate concentrated in vacuo. The crude material was purified by combi-flash chromatography, by eluting with 70% ethyl acetate in heptane, to afford tert-butyl 4-((3-(2-(2,6-dioxo-1-((2-(trimethylsilyl)ethoxy)methyl)piperidin-3-yl)-1-oxoisoindolin-4-yl)prop-2-yn-1-yl)oxy)piperidine-1-carboxylate (Intermediate 770, 2.5 g, 55%) as an off-white solid. Mass spec m/z 612.6 [M+H]⁺.

### Step 3

### Intermediate 771: 3-(1-oxo-4-(3-(piperidin-4-yloxy)prop-1-yn-1-yl)isoindolin-2-yl)piperidine-2,6-dione trifluoroacetate

To a cooled (0° C.) solution of 4-((3-(2-(2,6-dioxo-1-((2-(trimethylsilyl)ethoxy)methyl)piperidin-3-yl)-1-oxoisoindolin-4-yl)prop-2-yn-1-yl)oxy)piperidine-1-carboxylate (Intermediate 770, 2.50 g, 4.09 mmol) in dichloromethane (10 mL) was added trifluoroacetic acid (10 mL) and the reaction mixture was stirred at room temperature for 3 h. The reaction mixture was concentrated in vacuo and the crude solid was triturated with diethyl ether (2×10 mL) and dried in vacuo to afford 3-(1-oxo-4-(3-(4-piperidyloxy)prop-1-ynyl]isoindolin-2-yl)piperidine-2,6-dione trifluoroacetate (Intermediate 771, 2.0 g, 99%) as a white solid, which was used for next step without further purification. ¹H NMR (400 MHz, DMSO-d₆) δ ppm 1.70-1.73 (m, 2H), 2.01-2.09 (m, 4H), 2.45-2.52 (m, 2H), 2.50-2.80 (m, 1H), 3.01-3.06 (m, 2H), 3.15-3.20 (m, 2H), 4.29-4.35 (m, 1H), 4.48-4.54 (m, 2H), 5.02-5.10 (m, 1H), 5.23-5.28 (m, 1H), 7.56-7.60 (m, 1H), 7.72-7.80 (m, 1H), 8.67 (br s, 2H). Mass spec m/z 382.3 [M+H]⁺.

### Step 4

### Example 195: 6-(4-((3-(2-(2,6-dioxopiperidin-3-yl)-1-oxoisoindolin-4-yl)propa-1,2-dien-1-yl)oxy)piperidin-1-yl)-N-(2-((R)-1-methylpyrrolidin-2-yl)-1H-pyrrolo[3,2-c]pyridin-6-yl)nicotinamide

To a solution of 3-[1-oxo-4-[3-(4-piperidyloxy)prop-1-ynyl]isoindolin-2-yl]piperidine-2,6-dione trifluoroacetate (Intermediate 771, 1.13 g, 2.28 mmol) and tert-butyl (R)-6-(6-fluoronicotinamido)-2-(1-methylpyrrolidin-2-yl)-1H-pyrrolo[3,2-c]pyridine-1-carboxylate (Intermediate 562, 0.5 g, 1.14 mmol) in dimethyl sulfoxide (3 mL) was added N,N-diisopropylethylamine (0.81 mL, 4.55 mmol) and the reaction mixture was heated at 120° C. for 16 h. The reaction mixture was then concentrated in vacuo and the residue was diluted with ice cold water (100 mL). The resultant precipitate was collected by filtration, washed with n-pentane (50 mL), and dried in vacuo. The crude material was purified by preparative HPLC (Method A) to afford 6-(4-((3-(2-(2,6-dioxopiperidin-3-yl)-1-oxoisoindolin-4-yl)propa-1,2-dien-1-yl)oxy)piperidin-1-yl)-N-(2-((R)-1-methylpyrrolidin-2-yl)-1H-pyrrolo[3,2-c]pyridin-6-yl)nicotinamide (Example 195, 0.015 g, 1%) as an off-white solid. ¹H NMR (400 MHz, DMSO-d₆) δ ppm 1.49-1.52 (m, 2H), 1.77-1.90 (m, 6H), 2.11-2.2 (m, 4H), 2.22-2.3 (m, 1H), 2.45-2.55 (m, 3H), 2.85-2.95 (m, 1H), 3.01-3.08 (m, 1H), 3.81-3.91 (m, 1H), 4.05-4.15 (m, 2H), 4.35-4.41 (m, 1H), 4.47-4.55 (m, 3H), 5.13-5.17 (m, 1H), 6.37 (s, 1H), 6.86-6.95 (m, 1H), 7.54-7.67 (m, 3H), 7.81-7.88 (m, 2H), 8.01-8.13 (m, 2H), 8.47 (s, 1H), 8.75 (s, 1H), 10.26 (br s, 1H), 11.01 (br s, 1H), 11.38 (br s, 1H). Mass spec m/z 701.3 [M+H]⁺.

### Example 196 was Prepared Following Scheme 190

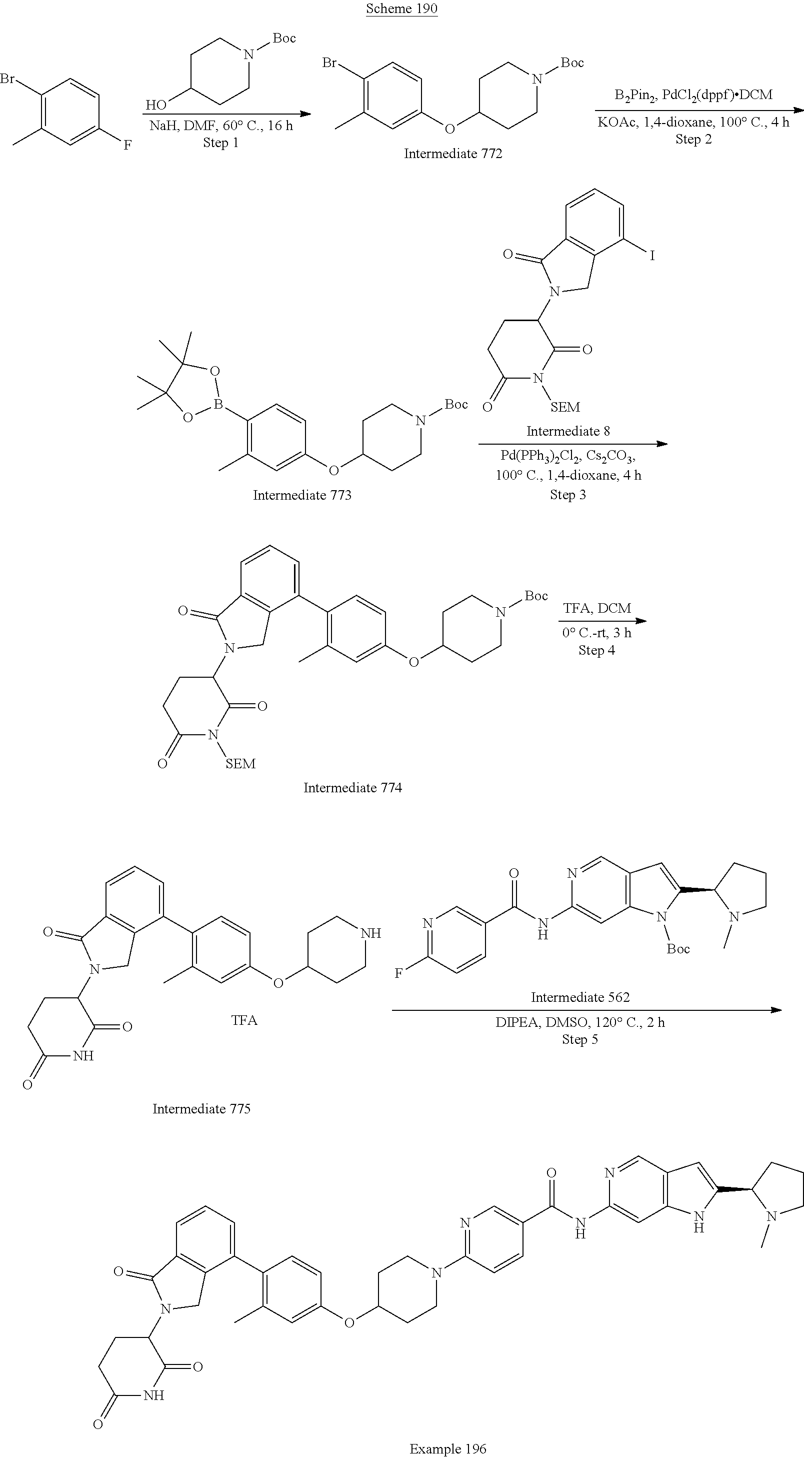

### Step 1

### Intermediate 772: tert-butyl 4-(4-bromo-3-methylphenoxy)piperidine-1-carboxylate

To a cooled (0° C.) solution of tert-butyl 4-hydroxypiperidine-1-carboxylate (CAS No. 109384-19-2, 10.0 g, 49.7 mmol) in dimethylformamide (100 mL) was added sodium hydride (60% in mineral oil, 3.97 g, 99.4 mmol) at and the reaction mixture was stirred for 30 min at 0° C. 1-Bromo-4-fluoro-2-methylbenzene (CAS No. 452-63-1, 11.3 g, 59.6 mmol) was then added at 0° C. and the reaction mixture was heated at 60° C. for 16 h. After completion, the reaction mixture was quenched with water (500 mL), the resultant precipitate was collected by filtration and dried in vacuo. The crude material was purified by combi-flash chromatography, by eluting with 30% ethyl acetate in heptane, to afford tert-butyl 4-(4-bromo-3-methylphenoxy)piperidine-1-carboxylate (Intermediate 772, 8.0 g, 43%) as a colorless liquid. ¹H NMR (400 MHz, DMSO-d₆) δ ppm 1.40 (s, 9H), 1.45-1.52 (m, 2H), 1.84-1.91 (m, 2H), 2.29 (s, 3H), 3.13-3.17 (m, 2H), 3.60-3.67 (m, 2H), 4.51-4.55 (m, 1H), 6.75 (dd, J=8.80, 2.80 Hz, 1H), 7.00 (d, J=2.80 Hz, 1H), 7.43 (d, J=8.8 Hz, 1H). Mass spec m/z 370.0 [M+H]⁺.

### Step 2

### Intermediate 773: tert-butyl 4-(3-methyl-4-(4,4,5,5-tetramethyl-1,3,2-dioxaborolan-2-yl)phenoxy)piperidine-1-carboxylate

To a solution of tert-butyl 4-(4-bromo-3-methylphenoxy)piperidine-1-carboxylate (Intermediate 772, 1.5 g, 3.0 mmol) 1,4-dioxane (15 mL) were added bis(pinacolato)diboron (1.2 g, 6.0 mmol) followed by potassium acetate (1.2 g, 12.0 mmol). The reaction mixture was purged with argon for 15 min then PdCl₂(dppf). CH₂Cl₂ (0.3 g, 0.3 mmol) was added. The reaction mixture was further purged with argon for 10 min and heated at 100° C. for 4 h. The reaction mixture was then concentrated in vacuo and the residue triturated with n-pentane (2×150 mL). The crude material was purified by combi-flash chromatography, by eluting with 40% ethyl acetate in heptane, to afford tert-butyl 4-(3-methyl-4-(4,4,5,5-tetramethyl-1,3,2-dioxaborolan-2-yl)phenoxy)piperidine-1-carboxylate (Intermediate 773, 1.2 g, 53%) as an off-white solid. Mass spec m/z 362.1 [M−56+H]⁺.

### Step 3

### Intermediate 774: tert-butyl 4-(4-(2-(2,6-dioxo-1-((2-(trimethylsilyl)ethoxy)methyl)piperidin-3-yl)-1-oxoisoindolin-4-yl)-3-methylphenoxy)piperidine-1-carboxylate

To a solution of tert-butyl 4-(3-methyl-4-(4,4,5,5-tetramethyl-1,3,2-dioxaborolan-2-yl)phenoxy)piperidine-1-carboxylate (Intermediate 773, 1.50 g, 3.0 mmol) in 1,4-dioxane (15 mL) was added 3-(4-iodo-1-oxoisoindolin-2-yl)-1-((2-(trimethylsilyl)ethoxy)methyl)piperidine-2,6-dione (Intermediate 8, 1.50 g, 3.0 mmol) followed by cesium carbonate (3.0 g, 9.0 mmol). The reaction mixture was purged with argon for 15 min then PdCl₂(dppf) (0.24 g, 0.3 mmol) was added. The reaction mixture was further purged with argon for 10 min and heated at 100° C. for 4 h. The reaction mixture was filtered through a celite bed and the filter pad was washed with ethyl acetate (100 mL). The filtrate was concentrated in vacuo and the crude material purified by combi-flash chromatography, by eluting with 80% ethyl acetate in heptane, to afford tert-butyl 4-(4-(2-(2,6-dioxo-1-((2-(trimethylsilyl)ethoxy)methyl)piperidin-3-yl)-1-oxoisoindolin-4-yl)-3-methylphenoxy)piperidine-1-carboxylate (Intermediate 774, 0.7 g, 35%) as a brown solid. ¹H NMR (400 MHz, DMSO-d₆) δ ppm −0.08 (s, 9H), 0.74-0.84 (m, 2H), 1.40 (s, 9H), 1.49-1.56 (m, 2H), 1.89-2.03 (m, 6H), 2.67-2.77 (m, 1H), 2.99-3.07 (m, 1H), 3.15-3.21 (m, 2H), 3.44-3.48 (m, 3H), 3.63-3.69 (m, 2H), 3.97-4.05 (m, 1H), 4.17-4.25 (m, 1H), 4.51-4.60 (m, 1H), 4.95-5.05 (m, 2H), 5.23-5.28 (m, 1H), 6.85-6.87 (m, 1H), 6.95 (s, 1H), 7.15 (d, J=8.80 Hz, 1H), 7.47 (d, J=7.60 Hz, 1H), 7.59 (t, J=7.6 Hz, 1H), 7.74 (d, J=7.2 Hz, 1H). Mass spec m/z 662.3 [M−H]⁻.

### Step 4

### Intermediate 775: 3-(4-(2-methyl-4-(piperidin-4-yloxy)phenyl)-1-oxoisoindolin-2-yl)piperidine-2,6-dione trifluoroacetate

To a cooled (0° C.) solution of tert-butyl 4-(4-(2-(2,6-dioxo-1-((2-(trimethylsilyl)ethoxy)methyl)piperidin-3-yl)-1-oxoisoindolin-4-yl)-3-methylphenoxy)piperidine-1-carboxylate (Intermediate 774, 0.60 g, 0.90 mmol) in dichloromethane (6 mL) was added trifluoroacetic acid (6.0 mL) and the reaction mixture was stirred at room temperature for 3 h. The reaction mixture was then concentrated in vacuo to afford 3-(4-(2-methyl-4-(piperidin-4-yloxy)phenyl)-1-oxoisoindolin-2-yl)piperidine-2,6-dione trifluoroacetate (Intermediate 775, 0.5 g) as a brown gummy solid, which was used for next step without further purification. Mass spec m/z 434.2 [M+H]⁺.

### Step 5

### Example 196: 6-(4-(4-(2-(2,6-dioxopiperidin-3-yl)-1-oxoisoindolin-4-yl)-3-methylphenoxy)piperidin-1-yl)-N-(2-((R)-1-methylpyrrolidin-2-yl)-1H-pyrrolo[3,2-c]pyridin-6-yl)nicotinamide

To a solution of 3-(4-(2-methyl-4-(piperidin-4-yloxy)phenyl)-1-oxoisoindolin-2-yl)piperidine-2,6-dione trifluoroacetate (Intermediate 775, 0.40 g, 0.73 mmol) and tert-butyl (R)-6-(6-fluoronicotinamido)-2-(1-methylpyrrolidin-2-yl)-1H-pyrrolo[3,2-c]pyridine-1-carboxylate (Intermediate 562, 0.32 g, 0.73 mmol) in dimethyl sulfoxide (5 mL) was added N,N-diisopropylethylamine (0.89 mL, 5.1 mmol) and the reaction mixture was heated at 120° C. for 2 h. The reaction mixture was concentrated in vacuo and the residue was diluted with ice cold water (20 mL). The resultant precipitate was collected by filtration, washed with n-pentane (50 mL), and dried in vacuo. The crude material was purified by preparative HPLC (Method A) to afford 6-(4-(4-(2-(2,6-dioxopiperidin-3-yl)-1-oxoisoindolin-4-yl)-3-methylphenoxy)piperidin-1-yl)-N-(2-((R)-1-methylpyrrolidin-2-yl)-1H-pyrrolo[3,2-c]pyridin-6-yl)nicotinamide (Example 196, 0.02 g, 4%) as an off-white solid. ¹H NMR (400 MHz, DMSO-d₆) δ ppm 1.61-1.71 (m, 2H), 1.75-1.99 (m, 4H), 2.01-2.19 (m, 4H), 2.12-2.18 (s, 3H), 2.21-2.27 (m, 1H), 2.48-2.49 (m, 2H), 2.52-2.56 (m, 2H), 2.83-2.91 (m, 1H), 3.12-3.17 (m, 1H), 3.25-3.29 (m, 1H), 3.48-3.53 (m, 2H), 4.07-4.14 (m, 3H), 4.18-4.23 (m, 1H), 4.20-4.26 (m, 1H), 5.09-5.14 (m, 1H), 6.37 (s, 1H), 6.90-6.95 (m, 2H), 7.00 (d, J=2.4 Hz, 1H), 7.15 (d, J=8.40 Hz, 1H), 7.47 (dd, J=7.60, 0.80 Hz, 1H), 7.59 (t, J=7.6 Hz, 1H), 7.74 (dd, J=7.60, 0.60 Hz, 1H), 8.15-8.20 (m, 2H), 8.48 (s, 1H), 8.81 (d, J=2.4 Hz, 1HHhhhH), 10.28 (s, 1H), 10.93 (s, 1H), 11.32 (s, 1H). Mass spec m/z 753.5 [M+H]⁺.

### Example 197 was Prepared Following Scheme 191

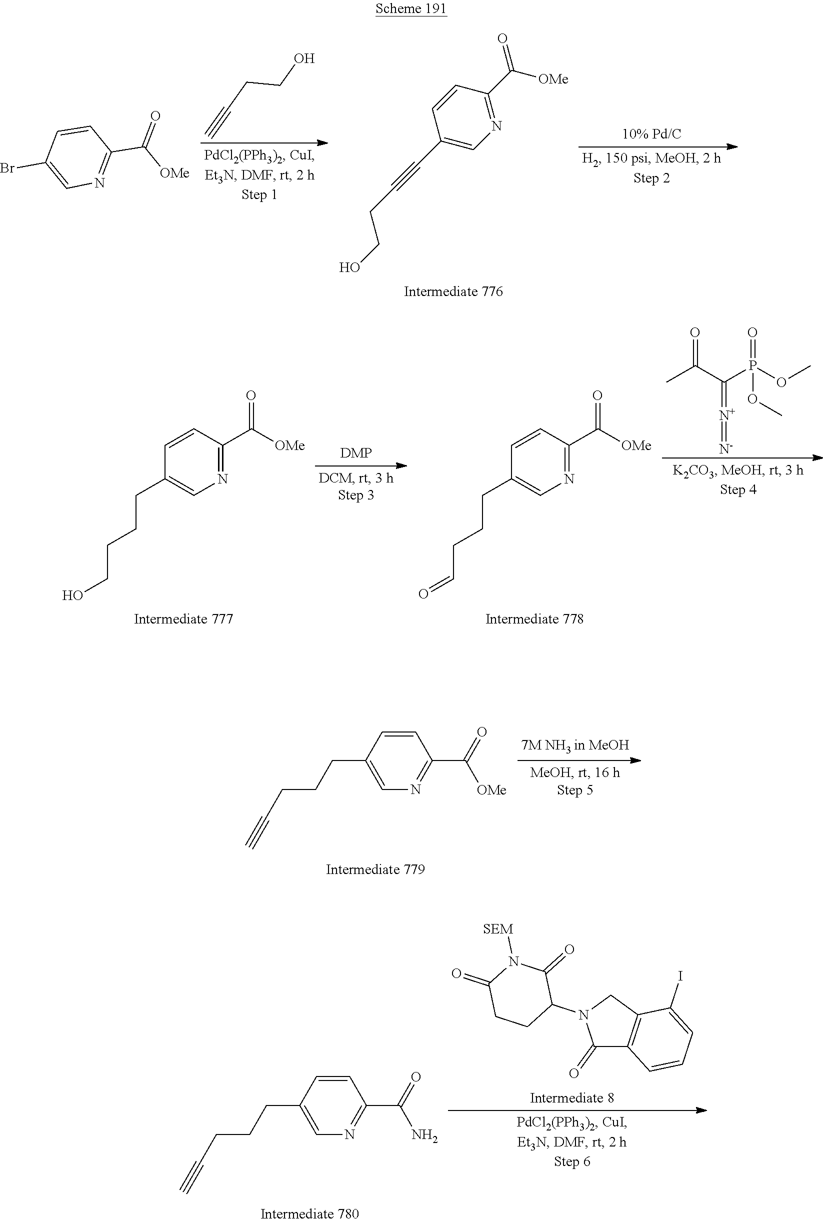

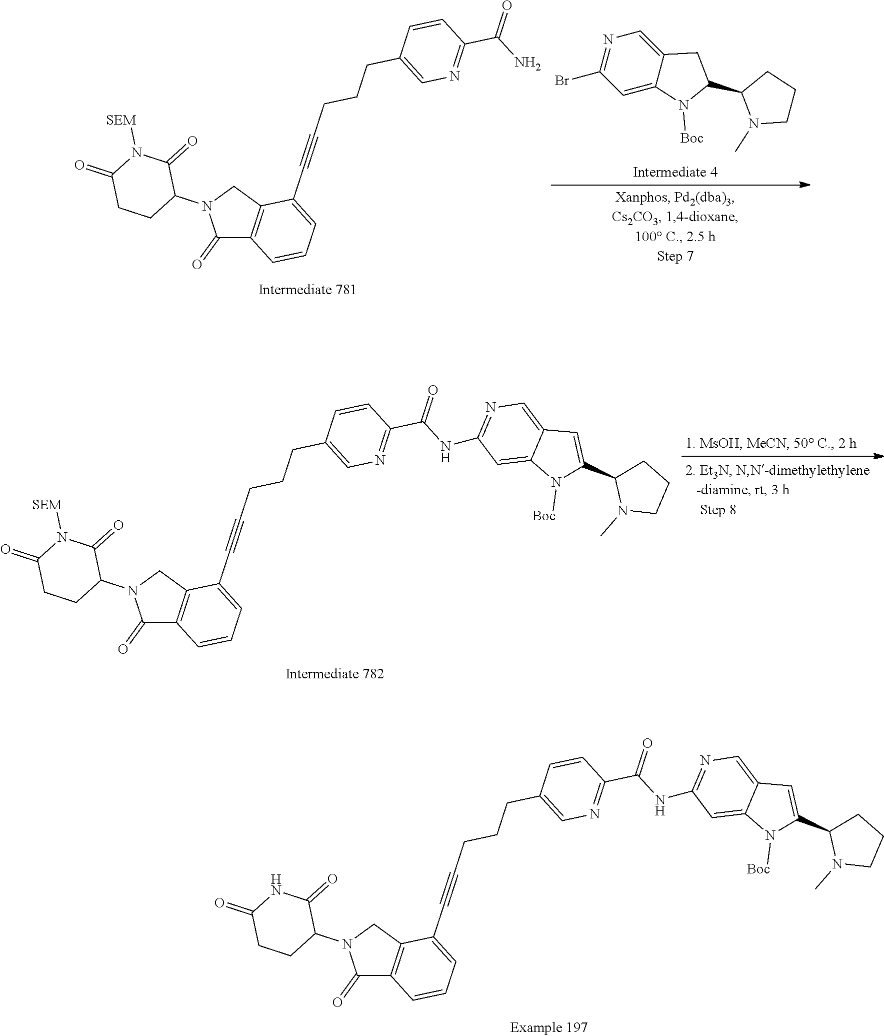 [SMILES: C[Si](C)(C)CCOCN1C(=O)CCC(N2Cc3c(C#CCCCc4ccc(C(N)=O)nc4)cccc3C2=O)C1=O ; CN1CCC[C@@H]1C1Cc2cnc(Br)cc2N1C(=O)OC(C)(C)C ; CN1CCC[C@@H]1c1cc2cnc(NC(=O)c3ccc(CCCC#Cc4cccc5c4CN(C4CCC(=O)N(COCC[Si](C)(C)C)C4=O)C5=O)cn3)cc2n1C(=O)OC(C)(C)C ; CN1CCC[C@@H]1c1cc2cnc(NC(=O)c3ccc(CCCC#Cc4cccc5c4CN(C4CCC(=O)NC4=O)C5=O)cn3)cc2n1C(=O)OC(C)(C)C]

### Step 1

### Intermediate 776: methyl 5-(4-hydroxybut-1-yn-1-yl)picolinate

To a solution of methyl 5-bromopicolinate (CAS No. 29682-15-3, 20.0 g, 92.6 mmol) in dimethylformamide (200 mL) was added but-3-yn-1-ol (CAS No. 927-74-2, 9.08 g, 130 mmol) followed by triethylamine (480 mL, 3426 mmol). The reaction mixture was purged with argon for 15 min then copper (I) iodide (2.64 g, 13.9 mmol) followed by PdCl₂(PPh₃)₂ (6.63 g, 9.25 mmol) were added. The reaction mixture was further purged with argon for 10 min and stirred at room temperature for 2 h. The reaction mixture was then quenched with ice cold water (600 mL) and extracted with ethyl acetate (3×200 mL). The combined organic layers were dried over anhydrous Na₂SO₄, filtered, and concentrated in vacuo. The crude material was purified by combi-flash chromatography, by eluting with 65% ethyl acetate in heptane, to afford methyl 5-(4-hydroxybut-1-yn-1-yl)picolinate (Intermediate 776, 18.0 g, 95%) as an off-white solid. ¹H NMR (400 MHz, DMSO-d₆) δ ppm 2.60-2.66 (m, 2H), 3.59-3.64 (m, 2H), 3.88 (s, 3H), 4.95-4.99 (m, 1H), 7.56-7.64 (m, 1H), 7.97-8.03 (m, 1H), 8.70 (s, 1H). Mass spec m/z 206.0 [M+H]⁺.

### Step 2

### Intermediate 777: methyl 5-(4-hydroxybutyl)picolinate

To a solution of methyl 5-(4-hydroxybut-1-yn-1-yl)picolinate (Intermediate 776, 18.0 g, 87.7 mmol) in methanol (360 mL) was added 10% Pd/C (8.0 g, 7.51 mmol) and the reaction mixture was stirred at room temperature for 16 h under 100 psi H₂ atmosphere. The reaction mixture was then filtered through a celite bed and the filter pad was washed with ethyl acetate (100 mL). The filtrate was concentrated in vacuo to afford methyl 5-(4-hydroxybutyl)picolinate (Intermediate 777, 18.0 g, 98%) as a brown liquid, which was used for the next step without further purification. Mass spec m/z 210.1 [M+H]⁺.

### Step 3

### Intermediate 778: methyl 5-(4-oxobutyl)picolinate

To a cooled (0° C.) solution of methyl 5-(4-hydroxybutyl)picolinate (Intermediate 777, 18.0 g, 86.9 mmol) in dichloromethane (270 mL) was added Dess-Martin periodinane (76.0 g, 174 mmol) and the reaction mixture was stirred at room temperature for 3 h. The reaction mixture was then quenched with saturated aqueous sodium thiosulphate solution (200 mL) and saturated aqueous sodium bicarbonate solution (200 mL), and extracted with dichloromethane (3×500 mL). The combined organic layers were dried over anhydrous Na₂SO₄, filtered, and concentrated in vacuo. The crude material was purified by combi-flash chromatography, by eluting with 45% ethyl acetate in heptane, to afford methyl 5-(4-oxobutyl)picolinate (Intermediate 778, 10.0 g, 48%) as a pale-yellow oil. ¹H NMR (400 MHz, DMSO-d₆) δ ppm 1.80-1.90 (m, 2H), 2.41-2.50 (m, 2H), 2.63-2.69 (m, 2H), 3.86 (s, 3H), 7.55-7.62 (m, 1H), 7.95-7.99 (m, 1H), 8.56 (s, 1H), 9.66 (s, 1H). Mass spec m/z 208.0 [M+H]⁺.

### Step 4

### Intermediate 779: methyl 5-(pent-4-yn-1-yl)picolinate

To a cooled (0° C.) solution of methyl 5-(4-oxobutyl)picolinate (Intermediate 778, 10.0 g, 48.3 mmol) in methanol (100 mL) was added potassium carbonate (13.3 g, 96.5 mmol) followed by dimethyl (1-diazo-2-oxopropyl)phosphonate (CAS No. 90965-06-3, 11.1 g, 57.9 mmol) and the reaction mixture was stirred at room temperature for 3 h. The reaction mixture was then diluted with water (500 mL) and extracted with diethyl ether (3×200 mL). The combined organic layers were dried over anhydrous Na₂SO₄, filtered, and concentrated in vacuo. The crude material was purified by combi-flash chromatography, by eluting with 10% ethyl acetate in heptane, to afford methyl 5-(pent-4-yn-1-yl)picolinate (Intermediate 779, 3.0 g, 31%) as a colourless oil. ¹H NMR (400 MHz, DMSO-d₆) δ ppm 1.74-1.81 (m, 2H), 2.15-2.20 (m, 2H), 2.74-2.78 (m, 2H), 2.82 (s, 1H), 3.86 (s, 3H), 7.80-7.84 (m, 1H), 7.96-7.99 (m, 1H), 8.56 (s, 1H). Mass spec m/z 204.1 [M+H]⁺.

### Step 5

### Intermediate 780: 5-(pent-4-yn-1-yl)picolinamide

To a solution of methyl 5-(pent-4-yn-1-yl)picolinate (Intermediate 779, 3.0 g, 14.8 mmol) in methanol (10 mL) was added 7M ammonia in methanol (60 mL) and the reaction mixture was stirred at room temperature for 16 h. The reaction mixture was then diluted with water (100 mL) and extracted with dichloromethane (3×100 mL). The combined organic layers were dried over anhydrous Na₂SO₄, filtered, and concentrated in vacuo to afford 5-(pent-4-yn-1-yl)picolinamide (Intermediate 780, 2.80 g) as an off-white solid, which was used for the next step without further purification. ¹H NMR (400 MHz, DMSO-d₆) δ ppm 1.74-1.82 (m, 2H), 2.16-2.19 (m, 2H), 2.74-2.78 (m, 2H), 2.83 (s, 1H), 7.56 (br s, 1H), 7.80-7.82 (m, 1H), 7.95 (d, J=8.0 Hz, 1H), 8.05 (br s, 1H), 8.47 (s, 1H). Mass spec m/z 189.3 [M+H]⁺.

### Step 6

### Intermediate 781: 5-(5-(2-(2,6-dioxo-1-((2-(trimethylsilyl)ethoxy)methyl)piperidin-3-yl)-1-oxoisoindolin-4-yl)pent-4-yn-1-yl)picolinamide

To a solution of 5-(pent-4-yn-1-yl)picolinamide (Intermediate 780, 2.80 g, 15.0 mmol) in dimethylformamide (2 mL) was added 3-(4-iodo-1-oxoisoindolin-2-yl)-1-((2-(trimethylsilyl)ethoxy)methyl)piperidine-2,6-dione (Intermediate 8, 8.20 g, 16.0 mmol) followed by triethylamine (77 mL, 550.0 mmol). The reaction mixture was purged with argon for 15 min, then copper (I) iodide (0.42 g, 2.20 mmol) followed by PdCl₂(PPh₃)₂ (1.10 g, 1.50 mmol) were added. The reaction mixture was further purged with argon for 10 min and stirred at room temperature for 2 h. The reaction mixture was then diluted with water (100 mL) and extracted with ethyl acetate (3×200 mL). The combined organic layers were dried over anhydrous Na₂SO₄ and concentrated in vacuo. The crude material was purified by combi-flash chromatography, by eluting with 60% ethyl acetate in heptane, to afford 5-(5-(2-(2,6-dioxo-1-((2-(trimethylsilyl)ethoxy)methyl)piperidin-3-yl)-1-oxoisoindolin-4-yl)pent-4-yn-1-yl)picolinamide (Intermediate 781, 5.50 g, 66%) as an off-white solid. ¹H NMR (400 MHz, DMSO-d₆) δ ppm −0.05-0.003 (m, 9H), 0.78-0.85 (m, 2H), 1.87-1.96 (m, 2H), 2.06-2.09 (m, 1H), 2.31-2.48 (m, 3H), 2.72-2.86 (m, 3H), 3.02-3.10 (s, 1H), 3.48-3.54 (s, 2H), 4.25-4.31 (m, 1H), 4.47-4.52 (m, 1H), 5.00-5.09 (m, 2H), 5.25-5.30 (m, 1H), 7.50-7.58 (m, 2H), 7.66 (b, J=7.20 Hz, 1H), 7.72 (b, J=8.00 Hz, 1H), 7.85 (d, J=8.00 Hz, 1H), 7.95-8.02 (m, 2H), 8.51 (s, 1H). Mass spec m/z 559.1 [M+H]⁺.

### Step 7

### Intermediate 782: tert-butyl 6-(5-(5-(2-(2,6-dioxo-1-((2-(trimethylsilyl)ethoxy)methyl)piperidin-3-yl)-1-oxoisoindolin-4-yl)pent-4-yn-1-yl)picolinamido)-2-((R)-1-methylpyrrolidin-2-yl)-1H-pyrrolo[3,2-c]pyridine-1-carboxylate

To a solution of 5-(5-(2-(2,6-dioxo-1-((2-(trimethylsilyl)ethoxy)methyl)piperidin-3-yl)-1-oxoisoindolin-4-yl)pent-4-yn-1-yl)picolinamide (Intermediate 781, 0.9 g, 1.60 mmol) in 1,4-dioxane (1.5 mL) was added tert-butyl (R)-6-bromo-2-(1-methylpyrrolidin-2-yl)-1H-pyrrolo[3,2-c]pyridine-1-carboxylate (Intermediate 4, 0.73 g, 1.96 mmol) followed by cesium carbonate (1.65 g, 4.81 mmol). The reaction mixture was purged with argon for 15 min, then Pd₂(dba)₃ (0.37 g, 0.40 mmol) and Xantphos (0.49 g, 0.72 mmol) were added. The reaction mixture was further purged with argon for 10 min and heated at 110° C. for 2.5 h. The reaction mixture was then concentrated in vacuo and the crude material was purified by combi-flash chromatography, by eluting with 90% ethyl acetate in heptane, to afford tert-butyl 6-(5-(5-(2-(2,6-dioxo-1-((2-(trimethylsilyl)ethoxy)methyl)piperidin-3-yl)-1-oxoisoindolin-4-yl)pent-4-yn-1-yl)picolinamido)-2-((R)-1-methylpyrrolidin-2-yl)-1H-pyrrolo[3,2-c]pyridine-1-carboxylate (Intermediate 782, 0.9 g, 33%) as a yellow solid. Mass spec m/z 860.1 [M+H]⁺.

### Step 8

### Example 197: 5-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)picolinamide

To a solution of tert-butyl 6-(5-(5-(2-(2,6-dioxo-1-((2-(trimethylsilyl)ethoxy)methyl)piperidin-3-yl)-1-oxoisoindolin-4-yl)pent-4-yn-1-yl)picolinamido)-2-((R)-1-methylpyrrolidin-2-yl)-1H-pyrrolo[3,2-c]pyridine-1-carboxylate (Intermediate 782, 0.90 g, 1.04 mmol) in acetonitrile (5 mL) was added methanesulfonic acid (0.13 mL, 2.09 mmol) and the reaction mixture was heated at 60° C. for 2 h. The reaction mixture was cooled to room temperature, then N,N′-dimethylethylenediamine (0.25 mL, 2.09 mmol) followed by triethylamine (2.93 mL, 20.9 mmol) were added and the reaction mixture was stirred at room temperature for 3 h. The reaction mixture was concentrated in vacuo and the crude material was purified by preparative HPLC (Method A) to afford 5-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)picolinamide (Example 197, 0.043 g, 7%) as an off-white solid. ¹H NMR (400 MHz, DMSO-d₆) δ ppm 1.75-2.03 (m, 7H), 2.16 (s, 3H), 2.24-2.33 (m, 1H), 2.40-2.47 (m, 2H), 2.52-2.63 (m, 3H), 2.86-2.95 (m, 3H), 3.10-3.17 (m, 1H), 4.33-4.38 (m, 1H), 4.47-4.51 (m, 1H), 5.11-5.16 (m, 1H), 6.41 (s, 1H), 7.52 (t, J=7.60 Hz, 1H), 7.65-7.72 (m, 2H), 7.97-8.00 (m, 1H), 8.15 (d, J=8.00 Hz, 1H), 8.27 (s, 1H), 8.49 (s, 1H), 8.66 (s, 1H), 10.28 (s, 1H), 11.02 (s, 1H), 11.45 (s, 1H). Mass spec m/z 630.2 [M+H]⁺.

### Example 198 was Prepared Following Scheme 192

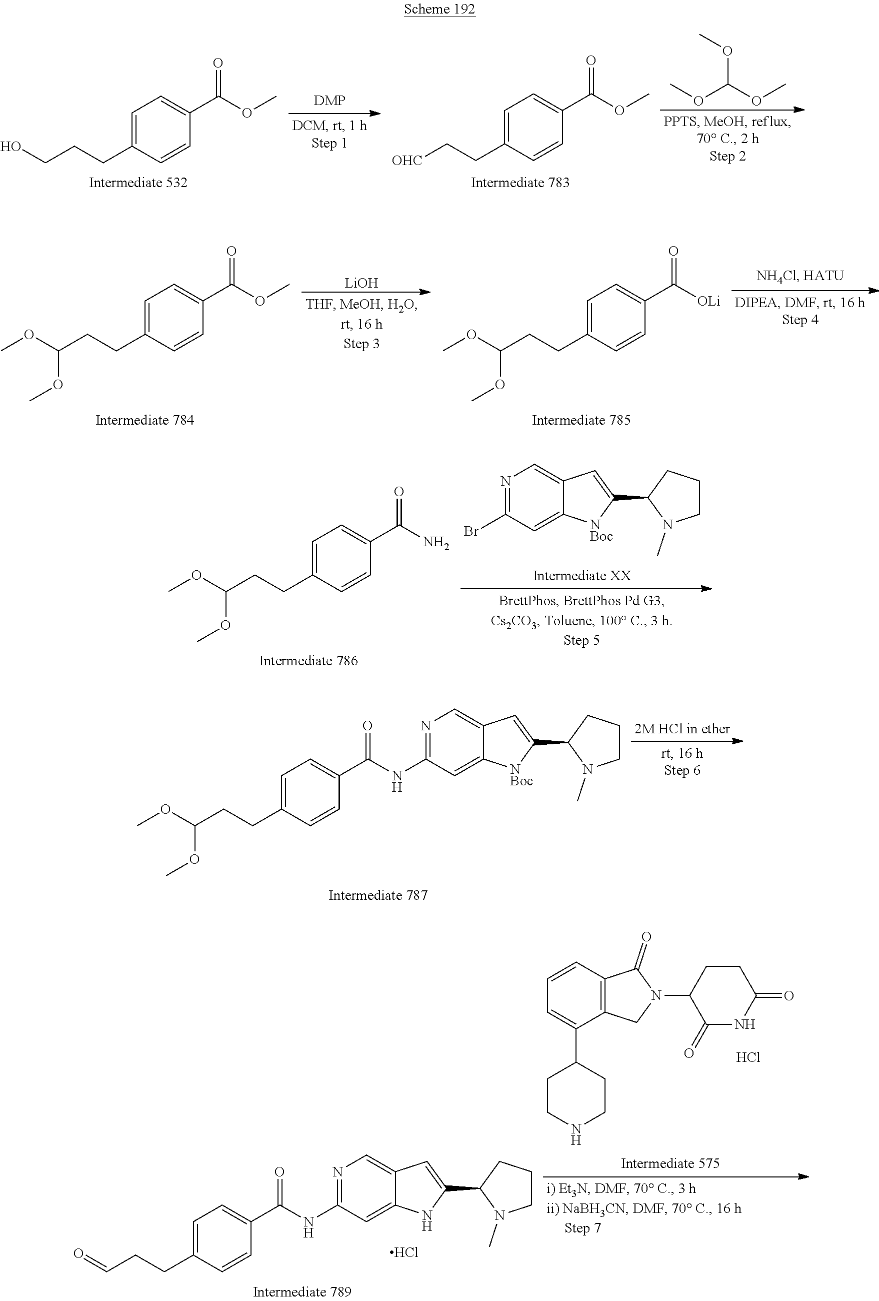

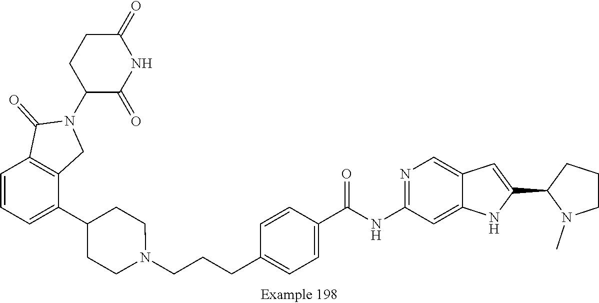 [SMILES: CN1CCC[C@@H]1c1cc2cnc(NC(=O)c3ccc(CCCN4CCC(c5cccc6c5CN(C5CCC(=O)NC5=O)C6=O)CC4)cc3)cc2[nH]1]

### Step 1

### Intermediate 783: methyl 4-(3-oxopropyl)benzoate

To a cooled (0° C.) solution of methyl 4-(3-hydroxypropyl)benzoate (Intermediate 532, 5.0 g, 25.7 mmol) in dichloromethane (60 mL) was added Dess-Martin periodinane (22.1 g, 51.5 mmol) and the reaction mixture was stirred at 0° C. for 1 h. The reaction mixture was then diluted with water (200 mL) and extracted with dichloromethane (2×500 mL). The combined organic layers were dried over anhydrous Na₂SO₄, filtered and concentrated in vacuo. The crude material was purified by combi-flash chromatography, by eluting with 50% ethyl acetate in heptane, to afford methyl 4-(3-oxopropyl)benzoate (Intermediate 783, 4.0 g, 81%) as an off-white solid. ¹H NMR (400 MHz, DMSO-d₆) δ ppm 2.81-2.83 (m, 2H), 2.84-2.97 (m, 2H), 3.85 (s, 3H), 7.37 (d, J=8.40 Hz, 2H), 7.88 (d, J=8.40, 2H), 9.71 (s, 1H). Mass spec m/z 191.0 [M−H]⁻.

### Step 2

### Intermediate 784: methyl 4-(3,3-dimethoxypropyl)benzoate

To a solution of methyl 4-(3-oxopropyl)benzoate (Intermediate 783, 1.80 g, 9.40 mmol) in methanol (20 mL) was added pyridinium p-toluenesulfonate (0.48 g, 1.90 mmol) and trimethyl orthoformate (CAS No. 149-73-5, 2.0 g, 19.0 mmol). The reaction mixture was heated at 70° C. for 2 h. The reaction mixture was then diluted with water (200 mL) and extracted with ethyl acetate (2×500 mL). The combined organic layers were dried over anhydrous Na₂SO₄, filtered and concentrated in vacuo. The crude material was purified by combi-flash chromatography, by eluting with 60% ethyl acetate in heptane, to afford methyl 4-(3,3-dimethoxypropyl)benzoate (Intermediate 784, 1.0 g, 45%) as an off-white solid. ¹H NMR (400 MHz, DMSO-d6) δ ppm 1.80-1.84 (m, 2H), 2.64-2.68 (m, 2H), 3.23 (s, 6H), 3.83 (s, 3H), 4.31-4.34 (m, 1H), 7.35 (d, J=8.40 Hz, 2H), 7.87 (d, J=8.40, 2H).

### Step 3

### Intermediate 785: Lithium salt of 4-(3,3-dimethoxypropyl)benzoic acid

To a solution of methyl 4-(3,3-dimethoxypropyl)benzoate (Intermediate 784, 2.0 g, 8.39 mmol) in a mixture of methanol (10 mL), tetrahydrofuran (10 mL) and water (5 mL) was added lithium hydroxide (0.61 g, 25.2 mmol) and the reaction mixture was stirred at room temperature for 16 h. The reaction mixture was concentrated in vacuo and triturated with dichloromethane to afford the lithium salt of 4-(3,3-dimethoxypropyl)benzoic acid (Intermediate 785, 1.50 g, 80%) as an off-white solid. ¹H NMR (400 MHz, DMSO-d₆) δ ppm 1.80-1.87 (m, 2H), 2.62-2.68 (m, 2H), 3.23 (s, 6H), 4.30-4.36 (m, 1H), 7.33 (d, J=7.20 Hz, 2H), 7.85 (d, J=7.40, 2H), 12.69 (br s, 1H). Mass spec m/z 223.1 [M−H]⁻. Step 4

### Intermediate 786: 4-(3,3-dimethoxypropyl)benzamide

To a cooled (0° C.) solution of lithium salt of 4-(3,3-dimethoxypropyl)benzoic acid (Intermediate 785, 1.50 g, 6.70 mmol) in dimethylformamide (15 mL) was added HATU (3.9 g, 10.0 mmol) and N,N-diisopropylethylamine (4.7 mL, 27.0 mmol). Ammonium chloride (1.2 mL, 33.0 mmol) was then added and reaction mixture was stirred at room temperature for 16 h. The reaction mixture was quenched with ice cold water (150 mL) and extracted with ethyl acetate (3×180 mL). The combined organic layers were dried over anhydrous Na₂SO₄, filtered and concentrated in vacuo. The crude material was purified by combi-flash chromatography, by eluting with 50% ethyl acetate in heptane, to afford 4-(3,3-dimethoxypropyl)benzamide (Intermediate 786, 1.30 g, 87%) as an off-white solid. ¹H NMR (400 MHz, DMSO-d₆) δ ppm 1.23-1.26 (m, 2H), 2.59-2.63 (m, 2H), 3.23 (s, 6H), 4.30-4.34 (m, 1H), 7.25-7.30 (m, 3H), 7.78 (d, J=8.0, 2H), 7.89 (br s, 1H).

### Step 5

### Intermediate 787: tert-butyl (R)-6-(4-(3,3-dimethoxypropyl)benzamido)-2-(1-methylpyrrolidin-2-yl)-1H-pyrrolo[3,2-c]pyridine-1-carboxylate

To a solution of 4-(3,3-dimethoxypropyl)benzamide (Intermediate 786, 0.60 g, 2.68 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 1.02 g, 2.68 mmol) and cesium carbonate (1.55 g, 8.06 mmol). The reaction mixture was purged with argon for 15 min then BrettPhos (0.22 g, 0.40 mmol) and BrettPhos Pd G3 (0.37 g, 0.40 mmol) were added. The reaction mixture was further purged with argon for 10 min and heated at 100° C. for 3 h. The reaction mixture was then concentrated in vacuo and the crude material purified by combi-flash chromatography, by eluting with 90% ethyl acetate in heptane, to afford tert-butyl (R)-6-(4-(3,3-dimethoxypropyl)benzamido)-2-(1-methylpyrrolidin-2-yl)-1H-pyrrolo[3,2-c]pyridine-1-carboxylate (Intermediate 787, 0.60 g, 21%) as a yellow solid. ¹H NMR (400 MHz, DMSO-d₆) δ ppm 1.20-1.29 (m, 2H), 1.60-1.79 (m, 12H), 1.82-1.89 (m, 2H), 2.30-2.39 (m, 5H), 2.65-2.69 (m, 2H), 3.24 (s, 6H), 3.34-3.37 (m, 1H), 6.74 (s, 1H), 7.34 (d, J=8.4 Hz, 2H), 7.98 (d, J=8.0 Hz, 2H), 8.59 (s, 1H), 8.92 (s, 1H), 10.61 (s, 1H). Mass spec m/z 523.4 [M+H]⁺.

### Step 6

### Intermediate 788: (R)—N-(2-(1-methylpyrrolidin-2-yl)-1H-pyrrolo[3,2-c]pyridin-6-yl)-4-(3-oxopropyl)benzamide hydrochloride

To a cooled (0° C.) solution of tert-butyl (R)-6-(4-(3,3-dimethoxypropyl)benzamido)-2-(1-methylpyrrolidin-2-yl)-1H-pyrrolo[3,2-c]pyridine-1-carboxylate (Intermediate 787, 0.40 g, 0.76 mmol) in diethyl ether (4 mL) was added 2M hydrochloric acid in diethyl ether (4 mL) and the reaction mixture was stirred at room temperature for 16 h. The reaction mixture was then concentrated in vacuo to afford (R)—N-(2-(1-methylpyrrolidin-2-yl)-1H-pyrrolo[3,2-c]pyridin-6-yl)-4-(3-oxopropyl)benzamide hydrochloride (Intermediate 788, 0.28 g, 89%) as a white solid, which was used for next step without further purification. Mass spec m/z 377.0 [M+H]⁺.

### Step 7

### Example 198: 4-(3-(4-(2-(2,6-dioxopiperidin-3-yl)-1-oxoisoindolin-4-yl)piperidin-1-yl)propyl)-N-(2-((R)-1-methylpyrrolidin-2-yl)-1H-pyrrolo[3,2-c]pyridin-6-yl)benzamide

To a solution of (R)—N-(2-(1-methylpyrrolidin-2-yl)-1H-pyrrolo[3,2-c]pyridin-6-yl)-4-(3-oxopropyl)benzamide hydrochloride (Intermediate 788, 0.10 g, 0.24 mmol) in dimethylformamide (2 mL) was added 3-(1-oxo-4-(piperidin-4-yl)isoindolin-2-yl)piperidine-2,6-dione hydrochloride (Intermediate 575, 0.08 g, 0.24 mmol) followed by triethylamine (0.17 mL, 1.21 mmol) and the reaction mixture was heated at 70° C. for 3 h. Sodium cyanoborohydride (0.06 g, 0.96 mmol) was then added and the reaction mixture was heated at 70° C. for 16 h. The reaction mixture was then poured into water (50 mL), the resultant precipitate was collected by filtration and dried in vacuo. The crude material was purified by preparative HPLC (Method B) to afford 4-(3-(4-(2-(2,6-dioxopiperidin-3-yl)-1-oxoisoindolin-4-yl)piperidin-1-yl)propyl)-N-(2-((R)-1-methylpyrrolidin-2-yl)-1H-pyrrolo[3,2-c]pyridin-6-yl)benzamide (Example 198, 0.01 g, 6%) as a yellow solid. ¹H NMR (400 MHz, DMSO-d₆) δ ppm 1.89-2.23 (m, 9H), 2.36-2.41 (m, 1H), 2.52-2.61 (m, 3H), 2.61-2.67 (m, 1H), 2.72-2.80 (m, 2H), 2.82-2.88 (m, 3H), 2.90-3.02 (m, 3H), 3.03-3.10 (m, 2H), 3.12-3.18 (m, 2H), 3.21-3.31 (m, 1H), 3.61-3.68 (m, 2H), 4.35-4.40 (m, 1H), 4.52-5.65 (m, 1H), 5.15-5.20 (m, 1H), 6.97 (s, 1H), 7.43-7.49 (s, 3H), 7.55 (t, J=7.6 Hz, 1H), 7.63-7.65 (s, 1H), 8.05 (d, J=8.40 Hz, 2H), 8.22 (s, 1H), 8.82 (s, 1H), 10.92 (br s, 1H), 11.02 (s, 1H), 12.02 (br s, 1H). Mass spec m/z 688.4 [M+H]⁺.

### Example 199 was Prepared Following Scheme 193

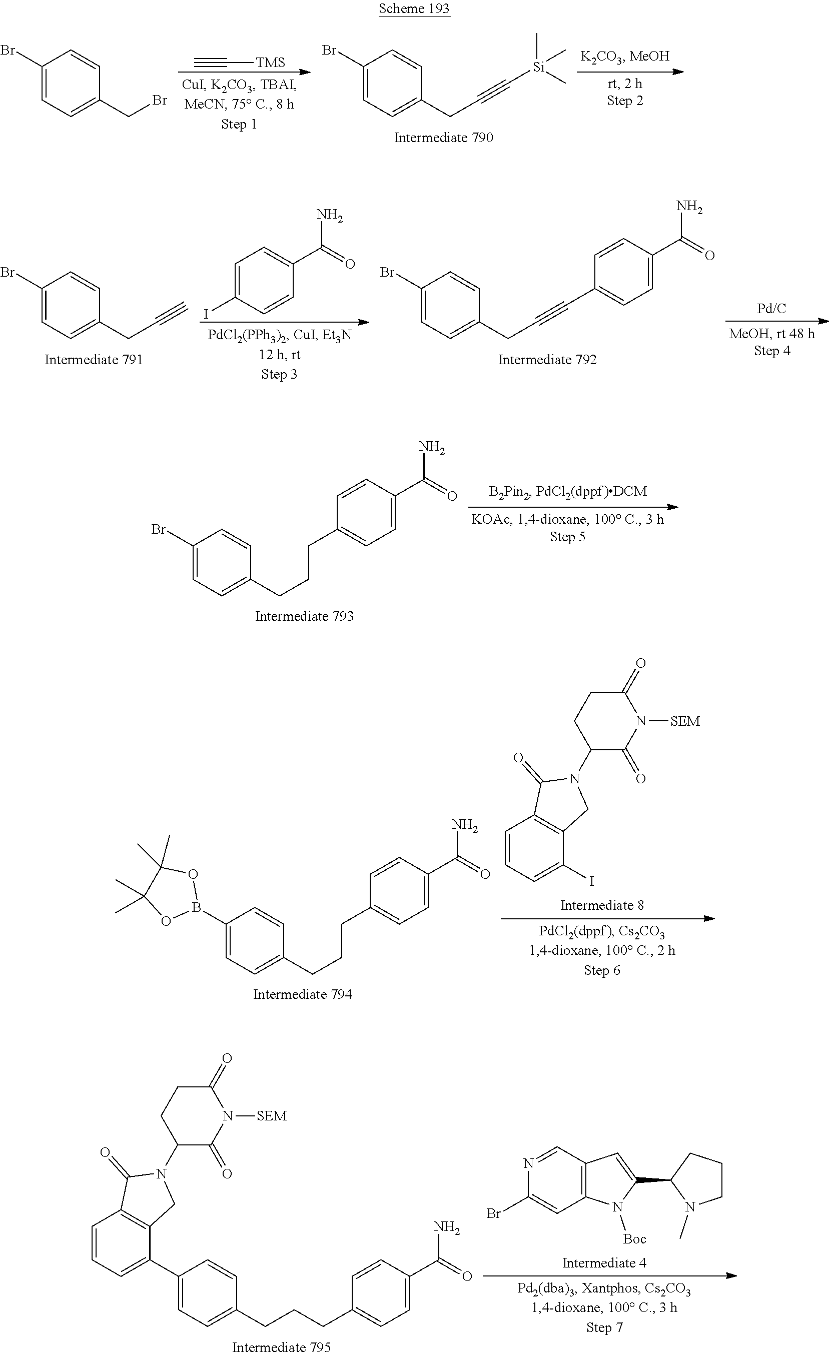

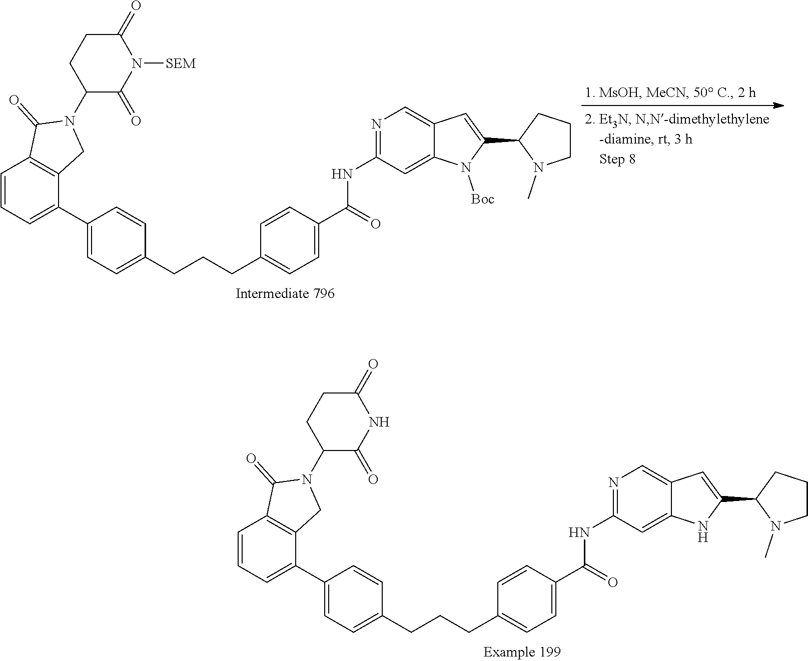 [SMILES: CN1CCC[C@@H]1c1cc2cnc(NC(=O)c3ccc(CCCc4ccc(-c5cccc6c5CN(C5CCC(=O)N(COCC[Si](C)(C)C)C5=O)C6=O)cc4)cc3)cc2n1C(=O)OC(C)(C)C ; CN1CCC[C@@H]1c1cc2cnc(NC(=O)c3ccc(CCCc4ccc(-c5cccc6c5CN(C5CCC(=O)NC5=O)C6=O)cc4)cc3)cc2[nH]1]

### Step 1

### Intermediate 790: (3-(4-bromophenyl)prop-1-yn-1-yl)trimethylsilane

To a solution of 1-bromo-4-(bromomethyl)benzene (CAS No. 589-15-1, 20.0 g, 78.4 mmol) in acetonitrile (200 mL) was added copper (I) iodide (14.94 g, 78.4 mmol) followed by potassium carbonate (13.01 g, 94.1 mmol), tetrabutylammonium iodide (29.56 g, 78.4 mmol) and trimethylsilylacetylene (22.6 mL, 157 mmol). The reaction mixture was heated at 75° C. for 8 h then concentrated in vacuo. The crude material was purified by combi-flash chromatography, by eluting with 2% ethyl acetate in heptane, to afford (3-(4-bromophenyl)prop-1-yn-1-yl)trimethylsilane (Intermediate 790, 8.0 g, 38%) as a yellow solid. ¹H NMR (400 MHz, DMSO-d₆) δ ppm 0.08 (s, 9H), 3.60 (s, 2H), 7.19-7.26 (m, 2H), 7.40-7.45 (m, 2H).

### Step 2

### Intermediate 791: 1-bromo-4-(prop-2-yn-1-yl)benzene

To a cooled (−10° C.) solution of (3-(4-bromophenyl)prop-1-yn-1-yl)trimethylsilane (Intermediate 790, 10 g, 37.4 mmol) in methanol (20 mL) was added potassium carbonate (10.34 g, 74.8 mmol) and the reaction mixture was stirred at −10° C. for 2 h. After completion, the reaction mixture was diluted with ice cold water (100 mL) and extracted with n-Pentane (2×200 mL). The combined organic layers were dried over anhydrous Na₂SO₄, filtered and concentrated in vacuo to afford 1-bromo-4-(prop-2-yn-1-yl)benzene (Intermediate 791, 5.0 g, 68%) as a yellow oil, which was used for the next step without further purification. ¹H NMR (400 MHz, DMSO-d₆) δ ppm 2.20 (s, 1H), 3.56 (s, 2H), 7.22-7.26 (m, 2H), 7.40-7.49 (m, 2H).

### Step 3

### Intermediate 792: 4-(3-(4-bromophenyl)prop-1-yn-1-yl)benzamide

To a solution of 1-bromo-4-(prop-2-yn-1-yl)benzene (Intermediate 791, 9.56 g, 48.1 mmol) in dimethylformamide (50 mL) was added 4-iodobenzamide (CAS No. 3956-07-8, 5.0 g, 19.2 mmol) followed by triethylamine (99.7 mL, 711 mmol). The reaction mixture was purged with argon for 15 min then copper (I) iodide (0.38 g, 1.92 mmol) and PdCl₂(PPh₃)₂ (1.42 g, 1.92 mmol) were added. The reaction mixture was further purged with argon for 10 min and stirred at room temperature for 3 h. The reaction mixture was then concentrated in vacuo and the crude material purified by combi-flash chromatography, by eluting with 60% ethyl acetate in heptane, to afford 4-(3-(4-bromophenyl)prop-1-yn-1-yl)benzamide (Intermediate 792, 5.6 g, 93%) as a green solid.

### Step 4

### Intermediate 793: 4-(3-(4-bromophenyl)propyl)benzamide

To a solution of 4-(3-(4-bromophenyl)prop-1-yn-1-yl)benzamide (Intermediate 792, 4.50 g, 13.6 mmol) in ethyl acetate (150 mL) was added 10% Pd/C (2.90 g, 27.2 mmol). The reaction mixture was stirred at room temperature for 48 h under 100 psi H₂ atmosphere. The reaction mixture was filtered through a celite bed and the filter pad was washed with methanol (100 mL). The filtrate was concentrated in vacuo to afford 4-(3-(4-bromophenyl)propyl)benzamide (Intermediate 793, 3.0 g, 69%) as a colourless oil, which was used for the next step without further purification. ¹H NMR (400 MHz, DMSO-d₆) δ ppm 1.84-1.92 (m, 2H), 2.53-2.67 (m, 4H), 7.15-7.30 (m, 5H), 7.46 (d, J=8.00 Hz, 2H), 7.78 (d, J=8.40 Hz, 2H), 7.88 (br s, 1H). Mass spec m/z 320.0 [M+H+2]⁺.

### Step 5

### Intermediate 794: 4-(3-(4-(4,4,5,5-tetramethyl-1,3,2-dioxaborolan-2-yl)phenyl)propyl)benzamide

To a solution of 4-(3-(4-bromophenyl)propyl)benzamide (Intermediate 793, 4.0 g, 12.6 mmol) 1,4-dioxane (40 mL) was added 4,4,4′,4′,5,5,5′,5′-octamethyl-2,2′-bi(1,3,2-dioxaborolane) (3.94 g, 15.1 mmol) followed by potassium acetate (3.74 g, 37.7 mmol). The reaction mixture was purged with argon for 15 min then PdCl₂(dppf). CH₂Cl₂ (1.57 g, 1.88 mmol) was added. The reaction mixture was further purged with argon for 10 min and heated at 100° C. for 3 h. The reaction mixture was concentrated in vacuo and the crude material was purified by combi-flash chromatography, by eluting with 70% ethyl acetate in heptane, to afford 4-(3-(4-(4,4,5,5-tetramethyl-1,3,2-dioxaborolan-2-yl)phenyl)propyl)benzamide (Intermediate 794, 3.0 g, 65%) as a brown solid. ¹H NMR (400 MHz, DMSO-d₆) δ ppm 1.06-1.28 (m, 12H), 1.86-1.99 (m, 2H), 2.56-2.66 (m, 4H), 7.15-7.30 (m, 7H), 7.79 (d, J=8.40 Hz, 2H), 7.88 (br s, 1H). Mass spec m/z 366.1 [M+H]⁺.

### Step 6

### Intermediate 795: 4-(3-(4-(2-(2,6-dioxo-1-((2-(trimethylsilyl)ethoxy)methyl)piperidin-3-yl)-1-oxoisoindolin-4-yl)phenyl)propyl)benzamide

To a solution of 4-(3-(4-(4,4,5,5-tetramethyl-1,3,2-dioxaborolan-2-yl)phenyl)propyl)benzamide (Intermediate 794, 1.0 g, 2.80 mmol) in 1,4-dioxane (13 mL) was added 3-(4-iodo-1-oxoisoindolin-2-yl)-1-((2-(trimethylsilyl)ethoxy)methyl)piperidine-2,6-dione (Intermediate 8, 1.40 g, 2.80 mmol) followed by cesium carbonate (2.70 g, 8.40 mmol). The reaction mixture was purged with argon for 15 min then PdCl₂(dppf) (0.23 g, 0.28 mmol) was added. The reaction mixture was further purged with argon for 10 min and heated at 100° C. for 2 h. The reaction mixture was concentrated in vacuo and the crude material was purified by combi-flash chromatography, by eluting with 80% ethyl acetate in heptane, to afford 4-(3-(4-(2-(2,6-dioxo-1-((2-(trimethylsilyl)ethoxy)methyl)piperidin-3-yl)-1-oxoisoindolin-4-yl)phenyl)propyl)benzamide (Intermediate 795, 0.4 0 g, 23%) as an off-white solid. Mass spec m/z 610.3 [M−H]⁻.

### Step 7

### Intermediate 796: tert-butyl 6-(4-(3-(4-(2-(2,6-dioxo-1-((2-(trimethylsilyl)ethoxy)methyl)piperidin-3-yl)-1-oxoisoindolin-4-yl)phenyl)propyl)benzamido)-2-((R)-1-methylpyrrolidin-2-yl)-1H-pyrrolo[3,2-c]pyridine-1-carboxylate

To a solution of 4-(3-(4-(2-(2,6-dioxo-1-((2-(trimethylsilyl)ethoxy)methyl)piperidin-3-yl)-1-oxoisoindolin-4-yl)phenyl)propyl)benzamide (Intermediate 795, 0.50 g, 0.81 mmol) in 1,4-dioxane (2 mL) was added tert-butyl (R)-6-bromo-2-(1-methylpyrrolidin-2-yl)-1H-pyrrolo[3,2-c]pyridine-1-carboxylate (Intermediate 4, 0.34 g, 0.89 mmol) followed by cesium carbonate (0.79 g, 2.45 mmol). The reaction mixture was purged with argon for 15 min then Pd₂(dba)₃ (0.11 g, 0.12 mmol) followed by Xantphos (0.14 g, 0.24 mmol) were added. The reaction mixture was further purged with argon for 10 min and heated at 100° C. for 3 h. The reaction mixture was filtered through a celite bed, the filter pad was washed with ethyl acetate (100 mL) and the filtrate concentrated in vacuo. The crude material was purified by combi-flash chromatography, by eluting with 80% ethyl acetate in heptane, to afford tert-butyl 6-(4-(3-(4-(2-(2,6-dioxo-1-((2-(trimethylsilyl)ethoxy)methyl)piperidin-3-yl)-1-oxoisoindolin-4-yl)phenyl)propyl)benzamido)-2-((R)-1-methylpyrrolidin-2-yl)-1H-pyrrolo[3,2-c]pyridine-1-carboxylate (Intermediate 796, 0.15 g, 10%) as a yellow solid. Mass spec m/z 911.5 [M+H]⁺.

### Step 8

### Example 199: 4-(3-(4-(2-(2,6-dioxopiperidin-3-yl)-1-oxoisoindolin-4-yl)phenyl)propyl)-N-(2-((R)-1-methylpyrrolidin-2-yl)-1H-pyrrolo[3,2-c]pyridin-6-yl)benzamide

To a solution of tert-butyl 6-(4-(3-(4-(2-(2,6-dioxo-1-((2-(trimethylsilyl)ethoxy)methyl)piperidin-3-yl)-1-oxoisoindolin-4-yl)phenyl)propyl)benzamido)-2-((R)-1-methylpyrrolidin-2-yl)-1H-pyrrolo[3,2-c]pyridine-1-carboxylate (Intermediate 796, 0.25 g, 0.27 mmol) in acetonitrile (3 mL) was added methanesulfonic acid (0.18 mL, 2.74 mmol) and the reaction mixture was heated at 60° C. for 2 h. The reaction mixture was cooled to room temperature, then N,N′-dimethylethylenediamine (0.16 mL, 1.37 mmol) followed by triethylamine (0.76 mL, 5.48 mmol) were added and the mixture was stirred at room temperature for 3 h. The reaction mixture was concentrated in vacuo and the resultant residue was diluted with water (50 mL). The resultant precipitate was collected by filtration and dried in vacuo. The crude solid was purified by preparative HPLC (Method A) to afford 4-(3-(4-(2-(2,6-dioxopiperidin-3-yl)-1-oxoisoindolin-4-yl)phenyl)propyl)-N-(2-((R)-1-methylpyrrolidin-2-yl)-1H-pyrrolo[3,2-c]pyridin-6-yl)benzamide (Example 199, 0.03 g, 17%) as an off-white solid. ¹H NMR (400 MHz, DMSO-d₆) δ ppm 1.65-2.02 (m, 6H), 2.13-2.18 (m, 4H), 2.23-2.28 (m, 1H), 2.41-2.48 (m, 2H), 2.53-2.61 (m, 2H), 2.67-2.73 (m, 3H), 2.84-2.91 (m, 1H), 3.13-3.17 (m, 1H), 4.39-4.44 (m, 1H), 4.60-4.64 (m, 1H), 5.11-5.16 (m, 1H), 6.38 (s, 1H), 7.36 (d, J=8.40 Hz, 4H), 7.53 (d, J=8.00 Hz, 2H), 7.62-7.45 (m, 3H), 7.75 (d, J=8.00 Hz, 2H), 8.18 (m, 1H), 8.49 (s, 1H), 10.37 (s, 1H), 10.96 (s, 1H), 11.36 (s, 1H). Mass spec m/z 681.3 [M+H]⁺.

### Example 200 was Prepared Following Scheme 194

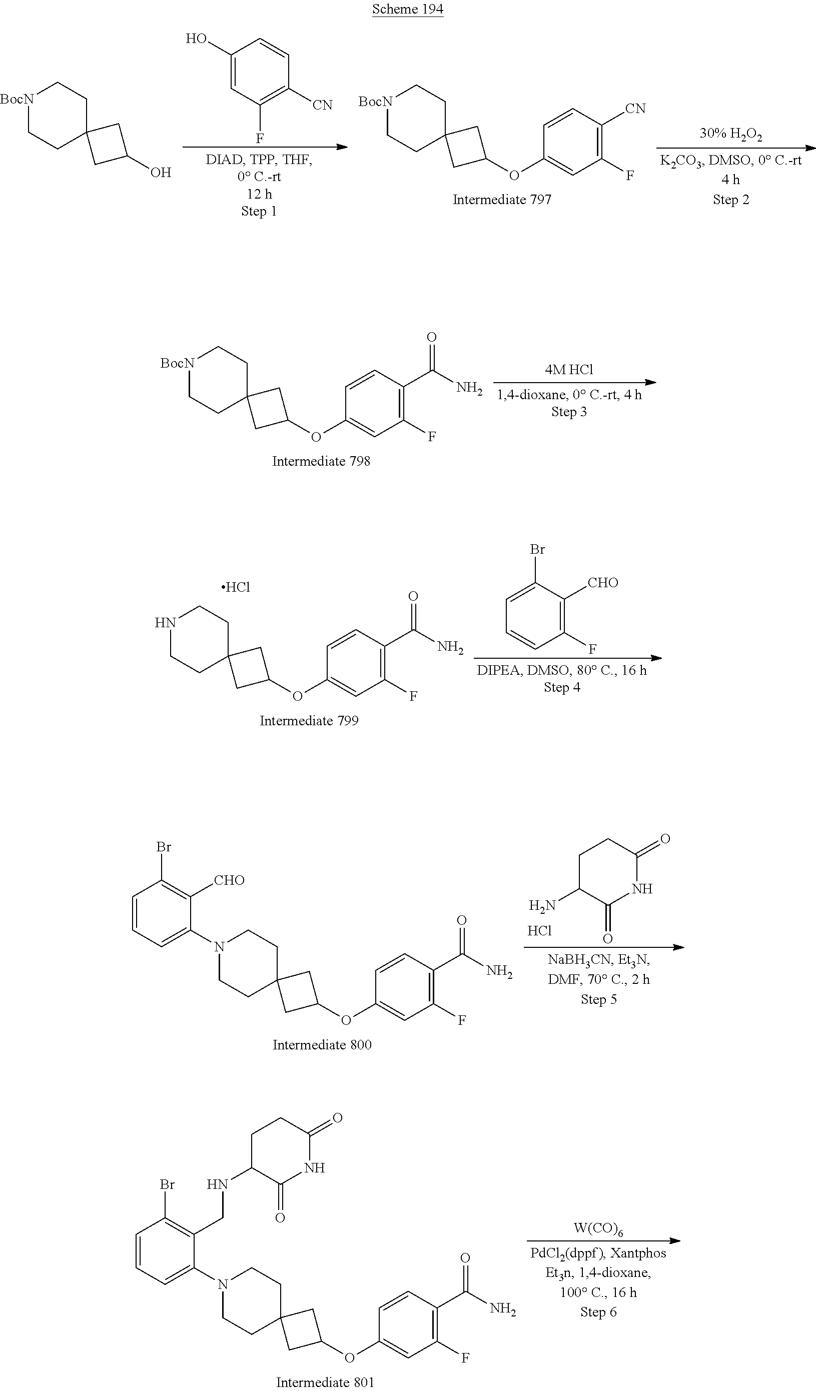

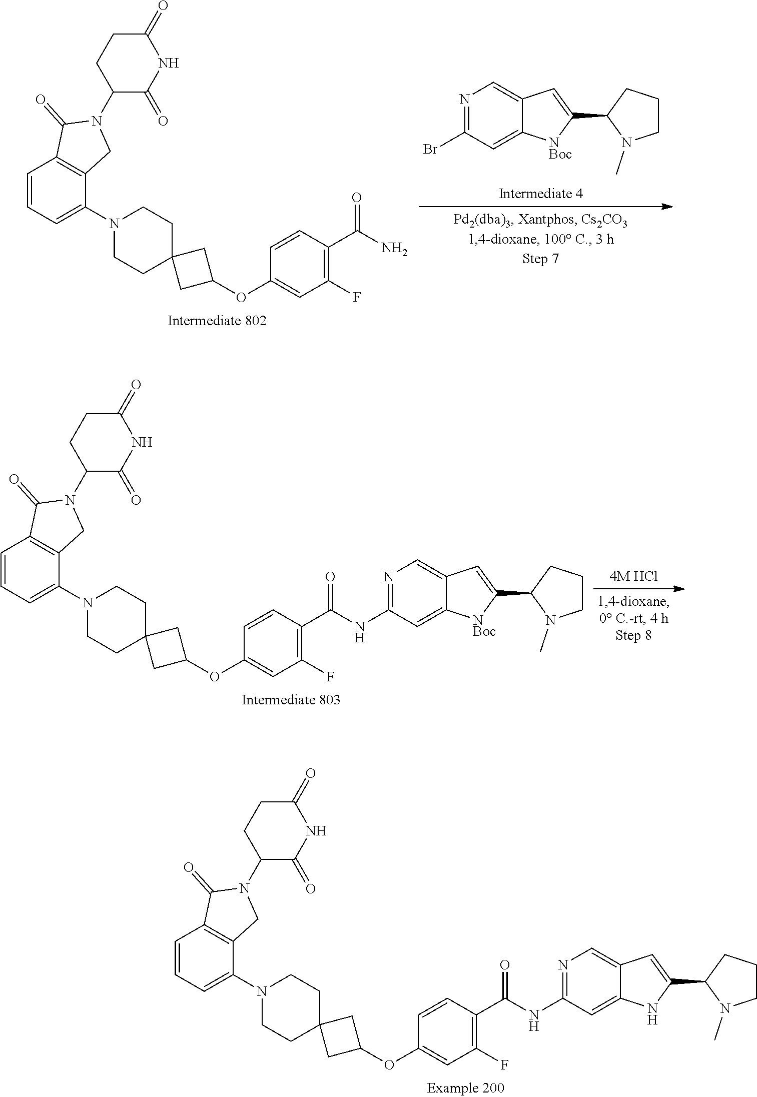 [SMILES: NC(=O)c1ccc(OC2CC3(CCN(c4cccc5c4CN(C4CCC(=O)NC4=O)C5=O)CC3)C2)cc1F ; CN1CCC[C@@H]1c1cc2cnc(Br)cc2n1C(=O)OC(C)(C)C ; CN1CCC[C@@H]1c1cc2cnc(NC(=O)c3ccc(OC4CC5(CCN(c6cccc7c6CN(C6CCC(=O)NC6=O)C7=O)CC5)C4)cc3F)cc2n1C(=O)OC(C)(C)C ; CN1CCC[C@@H]1c1cc2cnc(NC(=O)c3ccc(OC4CC5(CCN(c6cccc7c6CN(C6CCC(=O)NC6=O)C7=O)CC5)C4)cc3F)cc2[nH]1]

### Step 1

### Intermediate 797: tert-butyl 2-(4-cyano-3-fluorophenoxy)-7-azaspiro[3.5]nonane-7-carboxylate

To a cooled (0° C.) solution of tert-butyl 2-hydroxy-7-azaspiro[3.5]nonane-7-carboxylate (CAS No: 240401-28-9, 10.0 g, 41.4 mmol) in tetrahydrofuran (100 mL) was added 2-fluoro-4-hydroxybenzonitrile (CAS No. 82380-18-5, 5.11 g, 37.3 mmol) followed by triphenylphosphine (22.2 g, 82.9 mmol) and diisopropyl azodicarboxylate (12.5 mL, 62.2 mmol) and the reaction mixture was stirred at room temperature for 12 h. The reaction mixture was then quenched with ice cold water (200 mL) and extracted with ethyl acetate (2×200 mL). The combined organic layers were dried over anhydrous Na₂SO₄, filtered and concentrated in vacuo. The crude material was purified by combi-flash chromatography, by eluting with 30% ethyl acetate in heptane, to afford tert-butyl 2-(4-cyano-3-fluorophenoxy)-7-azaspiro[3.5]nonane-7-carboxylate (Intermediate 797, 15.0 g, 100%) as an off-white solid. ¹H NMR (400 MHz, DMSO-d₆) δ ppm 1.35 (s, 9H), 1.45-1.53 (m, 2H), 1.75-1.84 (m, 2H), 2.40-2.44 (m, 4H), 3.15-3.29 (m, 4H), 4.75-4.82 (m, 1H), 6.81 (d, J=8.80 Hz, 1H), 7.03 (d, J=11.60 Hz, 1H), 7.80 (d, J=7.60 Hz, 1H).

### Step 2

### Intermediate 798: tert-butyl 2-(4-carbamoyl-3-fluorophenoxy)-7-azaspiro[3.5]nonane-7-carboxylate

To a cooled (0° C.) solution of tert-butyl 2-(4-cyano-3-fluorophenoxy)-7-azaspiro[3.5]nonane-7-carboxylate (Intermediate 797, 14 g, 38.9 mmol) in dimethyl sulfoxide (140 mL) was added potassium carbonate (10.72, 77.7 mmol) followed by 30% hydrogen peroxide (3.57 mL, 117 mmol) and the reaction mixture was stirred at room temperature for 4 h. The reaction mixture was then quenched with ice cold water (500 mL), the resultant precipitate was collected by filtration and dried in vacuo to afford tert-butyl 2-(4-carbamoyl-3-fluorophenoxy)-7-azaspiro[3.5]nonane-7-carboxylate (Intermediate 798, 10 g, 68%) as a white solid, which was used for the next step without further purification. ¹H NMR (400 MHz, DMSO-d₆) δ ppm 1.38 (s, 9H), 1.45-1.53 (s, 4H), 1.74-1.83 (m, 2H), 2.40-2.44 (m, 2H), 3.19-3.29 (m, 4H), 4.80-4.48 (m, 1H), 6.73-6.77 (m, 2H), 7.42-7.48 (m, 2H), 7.64 (t, J=8.80 Hz, 1H). Mass spec m/z 377.0 [M−H]⁻.

### Step 3

### Intermediate 799: 4-((7-azaspiro[3.5]nonan-2-yl)oxy)-2-fluorobenzamide hydrochloride

To a cooled (0° C.) solution of tert-butyl 2-(4-carbamoyl-3-fluorophenoxy)-7-azaspiro[3.5]nonane-7-carboxylate (Intermediate 798, 10.0 g, 26.4 mmol) in 1,4-dioxane (100 mL) was added 4M hydrochloric acid in 1,4-dioxane (100 mL) and the reaction mixture was stirred at room temperature for 4 h. The reaction mixture was then concentrated in vacuo to afford 4-((7-azaspiro[3.5]nonan-2-yl)oxy)-2-fluorobenzamide hydrochloride (Intermediate 799, 8.0 g) as an off-white solid, which was used for the next step without further purification. ¹H NMR (400 MHz, DMSO-d₆) δ ppm 1.73-1.88 (s, 6H), 2.43-2.53 (m, 2H), 2.89-3.00 (m, 4H), 4.74-4.86 (m, 1H), 6.73-6.78 (m, 2H), 7.42-7.50 (m, 2H), 7.62-7.67 (m, 1H), 8.95 (br s, 2H). Mass spec m/z 279.1 [M+H]⁺.

### Step 4

### Intermediate 800: 4-((7-(3-bromo-2-formylphenyl)-7-azaspiro[3.5]nonan-2-yl)oxy)-2-fluorobenzamide

To a solution of 4-((7-azaspiro[3.5]nonan-2-yl)oxy)-2-fluorobenzamide hydrochloride (Intermediate 799, 4.0 g, 14.37 mmol) in dimethyl sulfoxide (40 mL) was added N,N-diisopropylethylamine (12.6 mL, 71.9 mmol) followed by 2-bromo-6-fluorobenzaldehyde (CAS No. 360575-28-6, 2.91 g, 14.4 mmol) and the reaction mixture was heated to 80° C. for 16 h. The reaction mixture was then quenched with water (250 mL) and extracted with ethyl acetate (2×400 mL). The combined organic layers were dried over anhydrous Na₂SO₄, filtered and concentrated in vacuo to afford 4-((7-(3-bromo-2-formylphenyl)-7-azaspiro[3.5]nonan-2-yl)oxy)-2-fluorobenzamide (Intermediate 800, 3.60 g, 54%) as a pale yellow solid, which was used for the next step without further purification. ¹H NMR (400 MHz, DMSO-d₆) δ ppm 1.70-1.88 (s, 6H), 2.45-2.49 (m, 2H), 2.89-2.93 (m, 2H), 2.95-3.02 (m, 2H), 4.83-4.88 (m, 1H), 6.74-6.80 (m, 2H), 7.22 (d, J=8.00 Hz, 1H), 7.31 (d, J=7.60 Hz, 1H), 7.37-7.49 (m, 3H), 7.65 (t, J=8.80 Hz, 1H), 10.04 (s, 1H). Mass spec m/z 462.9 [M+H+2]⁺.

### Step 5

### Intermediate 801: 4-((7-(3-bromo-2-(((2,6-dioxopiperidin-3-yl)amino)methyl)phenyl)-7-azaspiro[3.5]nonan-2-yl)oxy)-2-fluorobenzamide

To a solution of 4-((7-(3-bromo-2-formylphenyl)-7-azaspiro[3.5]nonan-2-yl)oxy)-2-fluorobenzamide (Intermediate 800, 3.50 g, 7.60 mmol) in dimethylformamide (35 mL) was added 3-aminopiperidine-2,6-dione hydrochloride (CAS No. 24666-56-6, 1.50 g, 9.10 mmol) followed by triethylamine (3.2 mL, 23 mmol) and the reaction mixture was heated at 70° C. for 3 h. Sodium cyanoborohydride (1.50 g, 23.0 mmol) was then added and the reaction mixture was heated at 70° C. for 2 h. The reaction mixture was quenched with water (100 mL), the resultant precipitate was collected by filtration and dried in vacuo to afford 4-((7-(3-bromo-2-(((2,6-dioxopiperidin-3-yl)amino)methyl)phenyl)-7-azaspiro[3.5]nonan-2-yl)oxy)-2-fluorobenzamide (Intermediate 801, 3.0 g, 69%) as a white solid, which was used for the next step without further purification. Mass spec m/z 574.8 [M+H+2]⁺.

### Step 6

### Intermediate 802: 4-((7-(2-(2,6-dioxopiperidin-3-yl)-1-oxoisoindolin-4-yl)-7-azaspiro[3.5]nonan-2-yl)oxy)-2-fluorobenzamide

To a solution of 4-((7-(3-bromo-2-(((2,6-dioxopiperidin-3-yl)amino)methyl)phenyl)-7-azaspiro[3.5]nonan-2-yl)oxy)-2-fluorobenzamide (Intermediate 801, 2.0 g, 3.48 mmol) in 1,4-dioxane (25 mL) was added triethylamine (1.47 mL, 10.5 mmol). The reaction mixture was purged with argon for 15 min then Xantphos (0.41 g, 0.69 mmol) followed by PdCl₂(dppf) (0.53 g, 0.69 mmol) followed by tungsten hexacarbonyl (0.63 g, 1.74 mmol) were added. The reaction mixture was further purged with argon for 10 min and heated at 100° C. for 16 h. The reaction mixture was filtered through a celite bed and the filter pad was washed with ethyl acetate (200 mL). The filtrate was concentrated in vacuo and the crude material was purified by combi-flash chromatography, by eluting with 100% ethyl acetate, to afford 4-((7-(2-(2,6-dioxopiperidin-3-yl)-1-oxoisoindolin-4-yl)-7-azaspiro[3.5]nonan-2-yl)oxy)-2-fluorobenzamide (Intermediate 802, 0.70 g, 38%) as a brown solid. ¹H NMR (400 MHz, DMSO-d₆) δ ppm 1.70-1.89 (s, 6H), 2.48-2.61 (m, 6H), 2.87-3.05 (m, 4H), 4.25-4.32 (m, 1H), 4.40-4.47 (m, 1H), 4.86-4.91 (m, 1H), 5.09-5.13 (m, 1H), 6.73-6.79 (m, 2H), 7.16 (d, J=8.40 Hz, 1H), 7.29 (d, J=7.20 Hz, 1H), 7.40-7.50 (m, 3H), 7.63-7.68 (m, 1H), 10.97 (s, 1H). Mass spec m/z 521.1 [M+H]⁺.

### Step 7

### Intermediate 803: tert-butyl 6-(4-((7-(2-(2,6-dioxopiperidin-3-yl)-1-oxoisoindolin-4-yl)-7-azaspiro[3.5]nonan-2-yl)oxy)-2-fluorobenzamido)-2-((R)-1-methylpyrrolidin-2-yl)-1H-pyrrolo[3,2-c]pyridine-1-carboxylate

To a solution of 4-((7-(2-(2,6-dioxopiperidin-3-yl)-1-oxoisoindolin-4-yl)-7-azaspiro[3.5]nonan-2-yl)oxy)-2-fluorobenzamide (Intermediate 802, 0.45 g, 0.86 mmol) in 1,4-dioxane (6 mL) was added tert-butyl (R)-6-bromo-2-(1-methylpyrrolidin-2-yl)-1H-pyrrolo[3,2-c]pyridine-1-carboxylate (Intermediate 4, 0.39 g, 1.03 mmol) followed by cesium carbonate (0.84 g, 2.59 mmol). The reaction mixture was purged with argon for 15 min then Pd₂(dba)₃ (0.12 g, 0.12 mmol) followed by Xantphos (0.15 g, 0.25 mmol) were added. The reaction mixture was further purged with argon for 10 min and heated at 100° C. for 3 h. The reaction mixture was filtered through a celite bed, the filter pad washed with ethyl acetate (100 mL) and the filtrate concentrated in vacuo. The crude material was purified by combi-flash chromatography, by eluting with 100% ethyl acetate, to afford tert-butyl 6-(4-((7-(2-(2,6-dioxopiperidin-3-yl)-1-oxoisoindolin-4-yl)-7-azaspiro[3.5]nonan-2-yl)oxy)-2-fluorobenzamido)-2-((R)-1-methylpyrrolidin-2-yl)-1H-pyrrolo[3,2-c]pyridine-1-carboxylate (Intermediate 803, 0.3 g, 42%) as an off-white solid. Mass spec m/z 820.5 [M+H]⁺.

### Step 8

### Example 200: 4-((7-(2-(2,6-dioxopiperidin-3-yl)-1-oxoisoindolin-4-yl)-7-azaspiro[3.5]nonan-2-yl)oxy)-2-fluoro-N-(2-((R)-1-methylpyrrolidin-2-yl)-1H-pyrrolo[3,2-c]pyridin-6-yl)benzamide

To a cooled (0° C.) solution of tert-butyl 6-(4-((7-(2-(2,6-dioxopiperidin-3-yl)-1-oxoisoindolin-4-yl)-7-azaspiro[3.5]nonan-2-yl)oxy)-2-fluorobenzamido)-2-((R)-1-methylpyrrolidin-2-yl)-1H-pyrrolo[3,2-c]pyridine-1-carboxylate (Intermediate 803, 0.30 g, 0.36 mmol) in 1,4-dioxane (3 mL) was added 4M hydrochloric acid in 1,4-dioxane (3.0 mL) and the reaction mixture was stirred at room temperature for 4 h. The reaction mixture was the concentrated in vacuo. The crude material was purified by preparative HPLC (Method A) to afford 4-((7-(2-(2,6-dioxopiperidin-3-yl)-1-oxoisoindolin-4-yl)-7-azaspiro[3.5]nonan-2-yl)oxy)-2-fluoro-N-(2-((R)-1-methylpyrrolidin-2-yl)-1H-pyrrolo[3,2-c]pyridin-6-yl)benzamide (Example 200, 0.02 g, 11%) as an off-white solid. ¹H NMR (400 MHz, DMSO-d₆) δ ppm 1.42-1.94 (m, 10H), 1.95-2.02 (m, 2H), 2.13-2.19 (m, 4H), 2.21-2.29 (m, 1H), 2.52-2.61 (m, 2H), 2.88-3.07 (m, 6H), 3.11-3.16 (m, 1H), 4.27-4.32 (m, 1H), 4.42-4.46 (m, 1H), 4.88-4.94 (m 1H), 5.07-5.13 (m, 1H), 6.39 (s, 1H), 6.82-6.87 (m, 2H), 7.18 (d, J=7.60 Hz, 1H), 7.30 (d, J=7.20 Hz, 1H), 7.43 (t, J=7.60 Hz, 1H), 7.74 (t, J=9.2 Hz, 1H), 8.19 (s, 1H), 8.46 (s, 1H), 9.98 (d, J=5.6 Hz, 1H), 10.99 (br s, 1H), 11.38 (s, 1H). Mass spec m/z 720.4 [M+H]⁺.

### Example 201 was Prepared Following Scheme 195

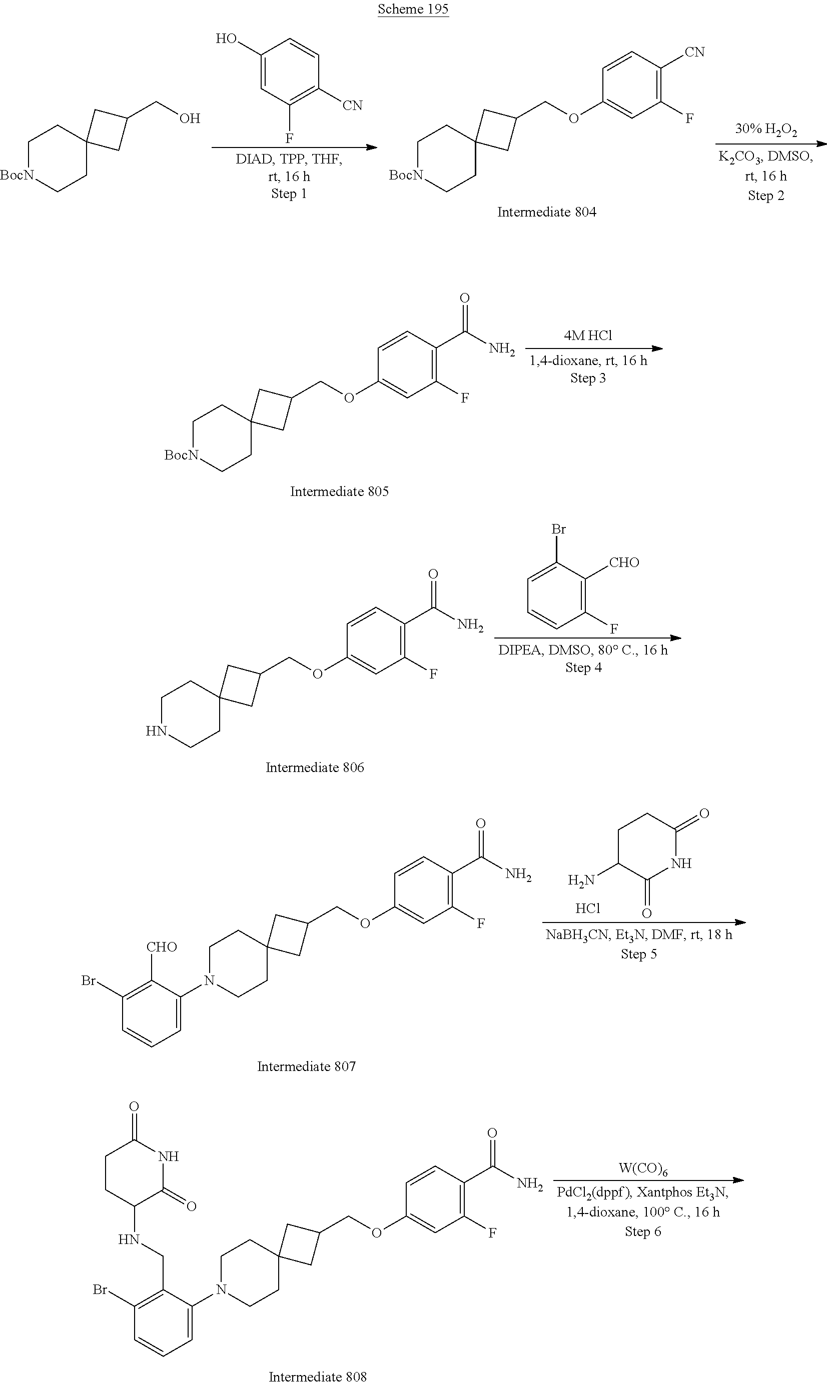

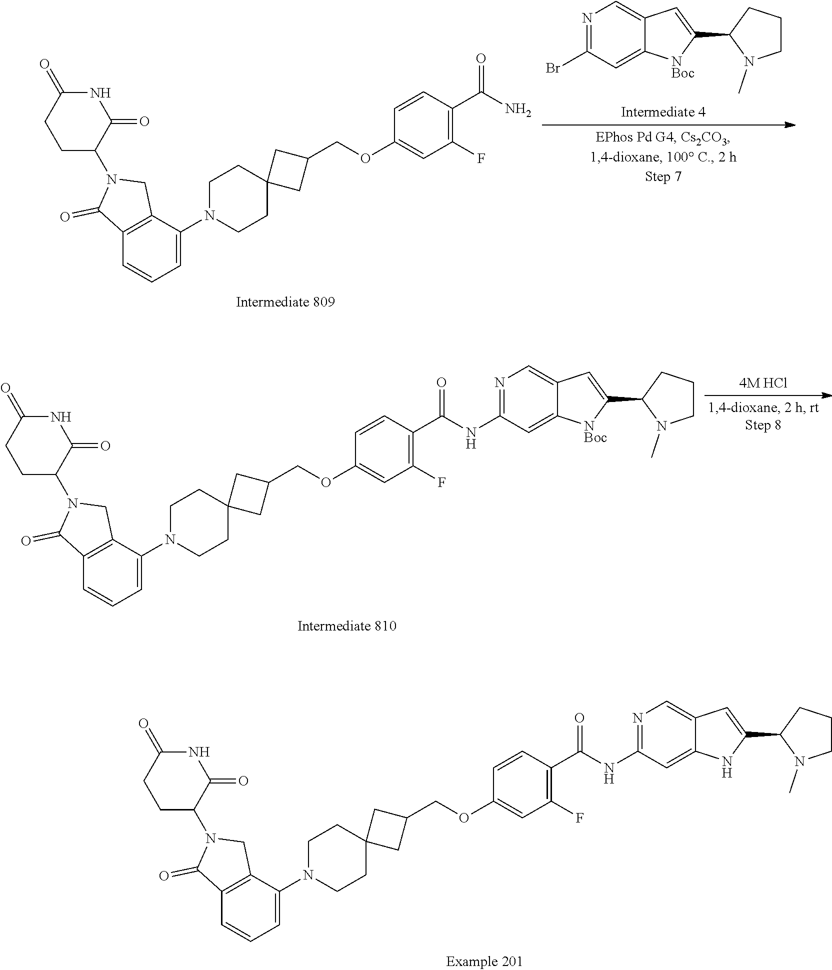 [SMILES: NC(=O)c1ccc(OCC2CC3(CCN(c4cccc5c4CN(C4CCC(=O)NC4=O)C5=O)CC3)C2)cc1F ; CN1CCC[C@@H]1c1cc2cnc(Br)cc2n1C(=O)OC(C)(C)C ; CN1CCC[C@@H]1c1cc2cnc(NC(=O)c3ccc(OCC4CC5(CCN(c6cccc7c6CN(C6CCC(=O)NC6=O)C7=O)CC5)C4)cc3F)cc2n1C(=O)OC(C)(C)C ; CN1CCC[C@@H]1c1cc2cnc(NC(=O)c3ccc(OCC4CC5(CCN(c6cccc7c6CN(C6CCC(=O)NC6=O)C7=O)CC5)C4)cc3F)cc2[nH]1]

### Step 1

### Intermediate 804: tert-butyl 2-((4-cyano-3-fluorophenoxy)methyl)-7-azaspiro[3.5]nonane-7-carboxylate

To a solution of 2-fluoro-4-hydroxybenzonitrile (CAS No. 82380-18-5, 0.18 g, 1.37 mmol) in tetrahydrofuran (15 mL) was added tert-butyl 2-(hydroxymethyl)-7-azaspiro[3.5]nonane-7-carboxylate (CAS No: 1356476-27-1, 0.7 g, 2.74 mmol), triphenylphosphine (0.73 g, 2.74 mmol) and diisopropyl azodicarboxylate (0.84 mL, 4.11 mmol) and the reaction mixture was stirred at room temperature for 16 h. The reaction mixture was quenched with saturated aqueous NH₄Cl solution (50 mL) and extracted with ethyl acetate (2×100 mL). The combined organic layers were dried over anhydrous Na₂SO₄, filtered and concentrated in vacuo. The crude material was purified by combi-flash chromatography, by eluting with 30% ethyl acetate in heptane, to afford tert-butyl 2-((4-cyano-3-fluorophenoxy)methyl)-7-azaspiro[3.5]nonane-7-carboxylate (Intermediate 804, 0.85 g, 83%) as a colorless viscous oil. ¹H NMR (400 MHz, DMSO-d₆) δ ppm 1.44 (s, 9H), 1.50-1.72 (m, 6H), 1.92-2.02 (m, 1H), 3.2-3.4 (m, 2H), 3.7-4.0 (m, 4H), 4.95-5.1 (m, 2H), 6.94-6.93 (m, 1H), 7.14-7.18 (m, 1H), 7.78-7.86 (m, 1H). Mass spec m/z 319.9 [M−56+H]⁺.

### Step 2

### Intermediate 805: tert-butyl 2-((4-carbamoyl-3-fluorophenoxy)methyl)-7-azaspiro[3.5]nonane-7-carboxylate

To a cooled (0° C.) solution of tert-butyl 2-((4-cyano-3-fluorophenoxy)methyl)-7-azaspiro[3.5]nonane-7-carboxylate (Intermediate 804, 1.50 g, 4.0 mmol) in dimethyl sulfoxide (10 mL) was added potassium carbonate (1.10 g, 8.0 mmol) followed by 30% hydrogen peroxide (1.2 mL, 12 mmol) and the reaction mixture was stirred at room temperature for 16 h. The reaction mixture was quenched with ice cold water (30 mL), the resultant precipitate was collected by filtration and dried in vacuo to afford tert-butyl 2-((4-carbamoyl-3-fluorophenoxy)methyl)-7-azaspiro[3.5]nonane-7-carboxylate (Intermediate 805, 0.5 g, 32%) as an off-white solid, which was used for the next step without further purification. ¹H NMR (400 MHz, DMSO-d₆) δ ppm 1.38-1.49 (m, 13H), 1.50-1.62 (m, 2H), 1.83-1.95 (m, 2H), 2.63-2.71 (m, 1H), 3.10-3.28 (m, 4H), 4.00-4.02 (m, 2H), 6.79-6.89 (m, 2H), 7.38-7.47 (m, 2H) 7.61-7.69 (m, 1H). Mass spec m/z 391.1 [M−H]⁻.

### Step 3

### Intermediate 806: 4-((7-azaspiro[3.5]nonan-2-yl)methoxy)-2-fluorobenzamide hydrochloride

To a solution of tert-butyl 2-((4-carbamoyl-3-fluorophenoxy)methyl)-7-azaspiro[3.5]nonane-7-carboxylate (Intermediate 805, 0.5 g, 1.27 mmol) in 1,4-dioxane (5 mL) was added 4M hydrochloric acid in 1,4-dioxane (5 mL) and the reaction mixture was stirred at room temperature for 16 h. The reaction mixture was then concentrated in vacuo to afford 4-((7-azaspiro[3.5]nonan-2-yl)methoxy)-2-fluorobenzamide hydrochloride (Intermediate 806, 0.3 g, 72%) as an off-white solid, which was used for the next step without further purification. ¹H NMR (400 MHz, DMSO-d₆) δ ppm 1.65-1.78 (m, 6H), 1.86-1.20 (m, 2H), 2.64-2.69 (m, 1H), 2.81-3.01 (m, 4H), 3.98-4.04 (m, 2H), 6.80-6.89 (m, 2H), 7.40-7.51 (m, 2H) 7.62-7.70 (m, 1H), 8.66 (br s, 2H). Mass spec m/z 293.1 [M+H]⁺.

### Step 4

### Intermediate 807: 4-((7-(3-bromo-2-formylphenyl)-7-azaspiro[3.5]nonan-2-yl)methoxy)-2-fluorobenzamide

To a solution of 4-((7-azaspiro[3.5]nonan-2-yl)methoxy)-2-fluorobenzamide; hydrochloride (Intermediate 806, 0.32 g, 0.97 mmol) in dimethylformamide (5 mL) were added potassium carbonate (0.40 g, 2.92 mmol) followed by 2-bromo-6-fluorobenzaldehyde (CAS No. 360575-28-6, 0.23 g, 1.16 mmol) and the reaction mixture was heated to 80° C. for 16 h. The reaction mixture was quenched with water (25 mL) and extracted with ethyl acetate (2×25 mL). The combined organic layers were dried over anhydrous Na₂SO₄, filtered and concentrated in vacuo. The crude material was purified by combi-flash chromatography, by eluting with 50% ethyl acetate in heptane, to afford 4-((7-(3-bromo-2-formylphenyl)-7-azaspiro[3.5]nonan-2-yl)methoxy)-2-fluorobenzamide (Intermediate 807, 0.4 g, 86%) as a yellow solid. ¹H NMR (400 MHz, DMSO-d₆) δ ppm 1.61-1.69 (m, 4H), 1.72-1.77 (m, 2H), 1.93-2.01 (m, 2H), 2.67-2.74 (m, 1H), 2.86-2.89 (m, 2H), 2.91-2.97 (m, 2H), 4.02-4.04 (m, 2H), 6.81-6.89 (m, 2H), 7.21 (d, J=8.00 Hz, 1H) 7.29 (d, J=8.00 Hz, 1H), 7.37-7.48 (m, 3H), 7.67 (t, J=8.80 Hz, 1H), 10.03 (s, 1H). Mass spec m/z 475.2 [M+H]⁺.

### Step 5

### Intermediate 808: 4-((7-(3-bromo-2-(((2,6-dioxopiperidin-3-yl)amino)methyl)phenyl)-7-azaspiro[3.5]nonan-2-yl)methoxy)-2-fluorobenzamide

To a solution of 4-((7-(3-bromo-2-formylphenyl)-7-azaspiro[3.5]nonan-2-yl)methoxy)-2-fluorobenzamide (Intermediate 807, 0.39 g, 0.82 mmol) in acetonitrile (5 mL) were added triethylamine (0.34 mL, 2.46 mmol) followed by 3-aminopiperidine-2,6-dione hydrochloride (CAS No. 24666-56-6, 0.12 g, 0.98 mmol) and the reaction mixture was stirred at room temperature for 2 h. Sodium cyanoborohydride (0.16 g, 2.46 mmol) was then added and the reaction mixture was stirred at room temperature for 16 h. The reaction mixture was then quenched with water (100 mL) and extracted with ethyl acetate (3×100 mL). The combined organic layers were dried over anhydrous Na₂SO₄, filtered, and concentrated in vacuo. The crude material was purified by combi-flash chromatography, by eluting with 70% ethyl acetate in heptane, to afford 4-((7-(3-bromo-2-(((2,6-dioxopiperidin-3-yl)amino)methyl)phenyl)-7-azaspiro[3.5]nonan-2-yl)methoxy)-2-fluorobenzamide (Intermediate 808, 0.4 g, 83%) as a yellow solid. ¹H NMR (400 MHz, DMSO-d₆) δ ppm 1.61-1.97 (m, 6H), 1.98-2.00 (m, 3H), 2.67-2.75 (m, 2H), 2.87-3.31 (m, 7H), 4.03-4.05 (m, 2H), 4.27-4.31 (m, 1H), 4.41-4.45 (m, 1H), 5.08-5.13 (m, 1H), 6.82-6.88 (m, 2H), 6.17 (d, J=7.60 Hz, 1H) 7.28 (d, J=8.40 Hz, 1H), 7.38-7.46 (m, 3H), 7.67 (t, J=8.40 Hz, 1H), 10.97 (s, 1H). Mass spec m/z 589.3 [M+H]⁺.

### Step 6

### Intermediate 809: 4-((7-(2-(2,6-dioxopiperidin-3-yl)-1-oxoisoindolin-4-yl)-7-azaspiro[3.5]nonan-2-yl)methoxy)-2-fluorobenzamide

To a solution of 4-((7-(3-bromo-2-(((2,6-dioxopiperidin-3-yl)amino)methyl)phenyl)-7-azaspiro[3.5]nonan-2-yl)methoxy)-2-fluorobenzamide (Intermediate 808, 0.38 g, 0.64 mmol) in 1,4-dioxane (2 mL) was added triethylamine (0.27 mL, 1.94 mmol). The reaction mixture was purged with argon for 15 min then Xantphos (0.07 g, 0.12 mmol) followed by PdCl₂(dppf) (0.09 g, 0.12 mmol) and tungsten hexacarbonyl (0.11 g, 0.32 mmol) were added. The reaction mixture was further purged with argon for 10 min and heated at 100° C. for 16 h. The reaction mixture was filtered through a celite bed and the filter pad was washed with ethyl acetate (30 mL). The filtrate was concentrated in vacuo to afford 4-((7-(2-(2,6-dioxopiperidin-3-yl)-1-oxoisoindolin-4-yl)-7-azaspiro[3.5]nonan-2-yl)methoxy)-2-fluorobenzamide (Intermediate 809, 0.35 g) as a brown solid, which was used for the next step without further purification.

Mass spec m/z 535.1 [M+H]⁺.

### Step 7

### Intermediate 810: tert-butyl 6-(4-((7-(2-(2,6-dioxopiperidin-3-yl)-1-oxoisoindolin-4-yl)-7-azaspiro[3.5]nonan-2-yl)methoxy)-2-fluorobenzamido)-2-((R)-1-methylpyrrolidin-2-yl)-1H-pyrrolo[3,2-c]pyridine-1-carboxylate

To a solution of 4-((7-(2-(2,6-dioxopiperidin-3-yl)-1-oxoisoindolin-4-yl)-7-azaspiro[3.5]nonan-2-yl)methoxy)-2-fluorobenzamide (Intermediate 809, 0.3 g, 0.56 mmol) in 1,4-dioxane (3 mL) were added tert-butyl (R)-6-bromo-2-(1-methylpyrrolidin-2-yl)-1H-pyrrolo[3,2-c]pyridine-1-carboxylate (Intermediate 4, 0.25 g, 0.67 mmol) followed by cesium carbonate (0.54 g, 1.68 mmol). The reaction mixture was purged with argon for 15 min then EPhos Pd G4 (0.08 g, 0.08 mmol) was added. The reaction mixture was further purged with argon for 10 min and heated at 100° C. for 2 h. The reaction mixture was filtered through a celite bed, the filter pad was washed with dichloromethane (50 mL) and the filtrate was concentrated in vacuo. The residue was triturated with n-heptane (10 mL) and dried in vacuo to afford tert-butyl 6-(4-((7-(2-(2,6-dioxopiperidin-3-yl)-1-oxoisoindolin-4-yl)-7-azaspiro[3.5]nonan-2-yl)methoxy)-2-fluorobenzamido)-2-((R)-1-methylpyrrolidin-2-yl)-1H-pyrrolo[3,2-c]pyridine-1-carboxylate (Intermediate 810, 0.4 g, 85%) as a brown solid, which was used for the next step without further purification. ¹H NMR (400 MHz, DMSO-d₆) δ ppm 1.51-1.69 (m, 17H), 1.98-2.03 (m, 2H), 2.29-2.42 (m, 7H), 2.71-2.74 (m, 2H), 2.90-2.93 (m, 2H), 2.99-3.02 (m, 2H), 3.10-3.15 (m, 2H), 3.89-3.93 (m, 2H), 4.04-4.11 (m, 2H), 4.26-4.32 (m, 1H), 4.40-4.45 (m, 1H), 5.02-5.07 (m, 1H), 6.60 (s, 1H), 6.74-6.97 (m, 2H), 7.15-7.18 (m, 1H), 7.27-7.30 (m, 1H), 7.40-7.41 (m, 2H), 7.69-7.75 (m, 1H), 8.56 (s, 1H), 8.91 (s, 1H), 10.26 (s, 1H). Mass spec m/z 834.3 [M+H]⁺.

### Step 8

### Example 201: 4-((7-(2-(2,6-dioxopiperidin-3-yl)-1-oxoisoindolin-4-yl)-7-azaspiro[3.5]nonan-2-yl)methoxy)-2-fluoro-N-(2-((R)-1-methylpyrrolidin-2-yl)-1H-pyrrolo[3,2-c]pyridin-6-yl)benzamide

To a solution of tert-butyl 6-(4-((7-(2-(2,6-dioxopiperidin-3-yl)-1-oxoisoindolin-4-yl)-7-azaspiro[3.5]nonan-2-yl)methoxy)-2-fluorobenzamido)-2-((R)-1-methylpyrrolidin-2-yl)-1H-pyrrolo[3,2-c]pyridine-1-carboxylate (Intermediate 810, 0.3 g, 0.35 mmol) in 1,4-dioxane (5 mL) was added 4M hydrochloric acid in 1,4-dioxane (5 mL) and the reaction mixture was stirred at room temperature for 2 h. The reaction mixture was then concentrated in vacuo. The crude material was purified by preparative HPLC (Method A) to afford 4-((7-(2-(2,6-dioxopiperidin-3-yl)-1-oxoisoindolin-4-yl)-7-azaspiro[3.5]nonan-2-yl)methoxy)-2-fluoro-N-(2-((R)-1-methylpyrrolidin-2-yl)-1H-pyrrolo[3,2-c]pyridin-6-yl)benzamide (Example 201, 0.052 g, 20%) as an off-white solid. ¹H NMR (400 MHz, DMSO-d₆) δ ppm 1.65-1.70 (m, 4H), 1.72-1.94 (m, 5H), 1.96-2.02 (m, 3H), 2.11-2.19 (m, 4H), 2.22-2.28 (m, 1H), 2.52-2.62 (m, 1H), 2.71-2.78 (m, 1H), 2.85-3.05 (m, 5H), 3.12-3.17 (m, 1H), 3.25-3.28 (m, 2H), 4.05-4.09 (m, 2H), 4.27-4.31 (m, 1H), 4.41-4.46 (m, 1H), 5.08-5.13 (m, 1H), 6.38 (s, 1H), 6.80-6.97 (m, 2H), 7.17 (d, J=7.6 Hz, 1H), 7.29 (d, J=7.20 Hz, 1H), 7.42 (t, J=7.60 Hz, 2H), 7.75 (t, J=8.40 Hz, 1H), 8.46 (s, 1H), 9.94 (d, J=5.60 Hz, 1H), 10.98 (s, 1H), 11.38 (s, 1H). Mass spec m/z 734.3 [M+H]⁺.

### Example 202 was Prepared Following Scheme 196

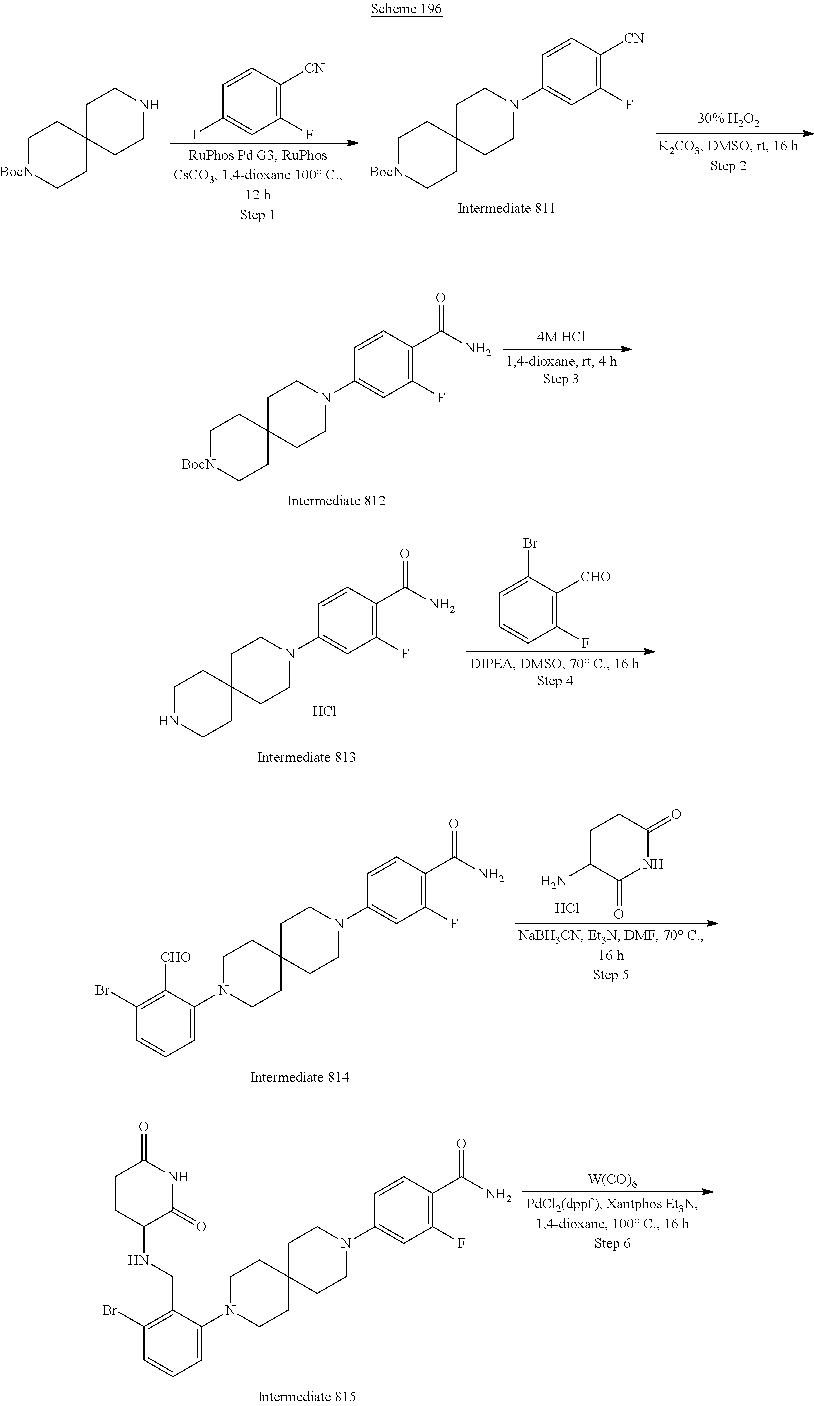

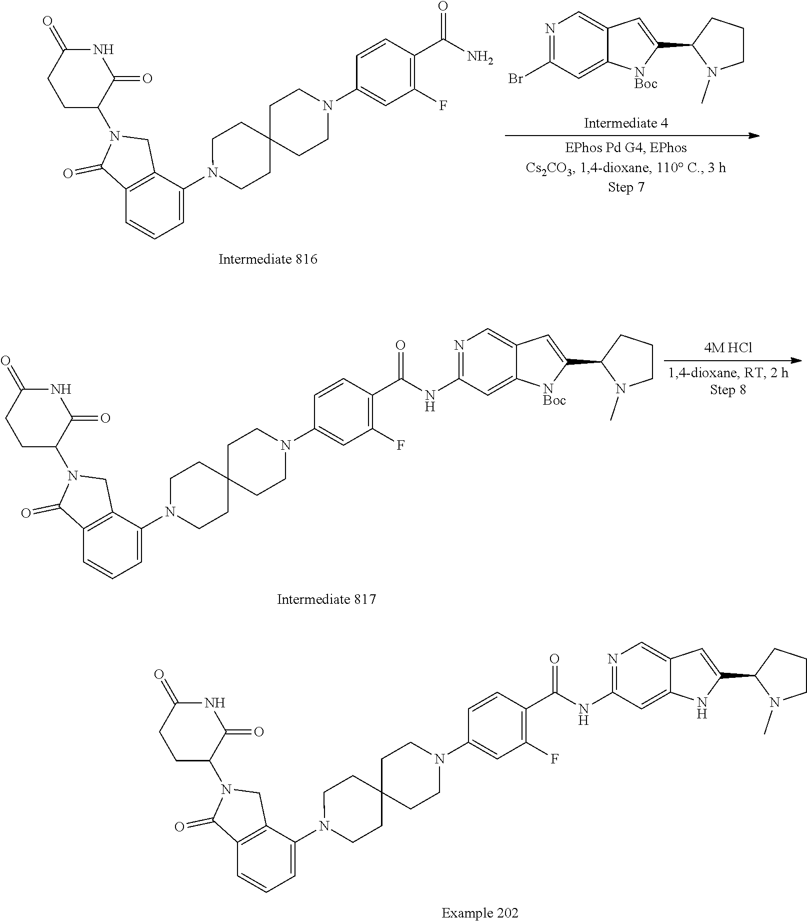 [SMILES: NC(=O)c1ccc(N2CCC3(CC2)CCN(c2cccc4c2CN(C2CCC(=O)NC2=O)C4=O)CC3)cc1F ; CN1CCC[C@@H]1c1cc2cnc(Br)cc2n1C(=O)OC(C)(C)C ; CN1CCC[C@@H]1c1cc2cnc(NC(=O)c3ccc(N4CCC5(CC4)CCN(c4cccc6c4CN(C4CCC(=O)NC4=O)C6=O)CC5)cc3F)cc2n1C(=O)OC(C)(C)C ; CN1CCC[C@@H]1c1cc2cnc(NC(=O)c3ccc(N4CCC5(CC4)CCN(c4cccc6c4CN(C4CCC(=O)NC4=O)C6=O)CC5)cc3F)cc2[nH]1]

### Step 1

### Intermediate 811: tert-butyl 9-(4-cyano-3-fluorophenyl)-3,9-diazaspiro[5.5]undecane-3-carboxylate

To a solution of tert-butyl 3,9-diazaspiro[5.5]undecane-3-carboxylate (CAS No. 173405-78-2, 15 g, 59.0 mmol) in 1,4-dioxane (150 mL) were added 2-fluoro-4-iodobenzonitrile (CAS No. 137553-42-5, 19.6 g, 77.8 mmol) followed by cesium carbonate (57.7 g, 177 mmol). The reaction mixture was purged with argon for 15 min then RuPhos Pd G3 (7.78 g, 8.84 mmol) followed by RuPhos (4.21 g, 8.84 mmol) at were added. The reaction mixture was further purged with argon for 10 min and heated at 100° C. for 12 h. The reaction mixture was filtered through a celite bed and the filter pad was washed with ethyl acetate (200 mL). The filtrate was concentrated in vacuo and the crude material purified by combi-flash chromatography, by eluting with 5% ethyl acetate in heptane, to afford tert-butyl 9-(4-cyano-3-fluorophenyl)-3,9-diazaspiro[5.5]undecane-3-carboxylate (Intermediate 811, 14 g, 63%) as a brown solid. ¹H NMR (400 MHz, DMSO-d₆) δ ppm 1.40-1.53 (m, 13H), 1.55-1.65 (m, 4H), 3.29-3.49 (m, 8H), 6.52 (d, J=9.20 Hz, 1H), 6.60 (d, J=8.0 Hz, 1H), 7.37 (t, J=8.80 Hz, 1H).

### Step 2

### Intermediate 812: tert-butyl 9-(4-carbamoyl-3-fluorophenyl)-3,9-diazaspiro[5.5]undecane-3-carboxylate

To a cooled (0° C.) solution of tert-butyl 9-(4-cyano-3-fluorophenyl)-3,9-diazaspiro[5.5]undecane-3-carboxylate (Intermediate 811, 14 g, 37.5 mmol) in dimethyl sulfoxide (150 mL) were added potassium carbonate (13.8, 99.8 mmol) followed by 30% hydrogen peroxide (12.3 mL, 401 mmol) and the reaction mixture was stirred at room temperature for 16 h. The reaction mixture was quenched with ice cold water (200 mL), the resultant precipitate was collected by filtration and dried in vacuo to afford tert-butyl 9-(4-carbamoyl-3-fluorophenyl)-3,9-diazaspiro[5.5]undecane-3-carboxylate (Intermediate 812, 10.0 g, 68%) as an off-white solid, which was used for the next step without further purification. ¹H NMR (400 MHz, DMSO-d₆) δ ppm 1.40-1.53 (m, 13H), 1.57-1.68 (m, 4H), 3.29-3.28 (m, 4H), 3.38-3.47 (m, 4H), 5.58 (br s, 1H), 6.43-6.66 (m, 2H), 6.68-6.71 (m, 1H), 7.96 (t, J=9.20 Hz, 1H).

### Step 3

### Intermediate 813: 2-fluoro-4-(3,9-diazaspiro[5.5]undecan-3-yl)benzamide hydrochloride

To a solution of tert-butyl 9-(4-carbamoyl-3-fluorophenyl)-3,9-diazaspiro[5.5]undecane-3-carboxylate (Intermediate 812, 5.0 g, 12.8 mmol) in dichloromethane (10 mL) was added 4M hydrochloric acid in 1,4-dioxane (50 mL). The reaction mixture was stirred at room temperature for 4 h. The reaction mixture was then concentrated in vacuo to afford 2-fluoro-4-(3,9-diazaspiro[5.5]undecan-3-yl)benzamide hydrochloride (Intermediate 813, 4.0 g) as an off-white solid, which was used for the next step without further purification. ¹H NMR (400 MHz, DMSO-d₆) δ ppm 1.51-1.65 (m, 8H), 2.95-3.06 (m, 4H), 3.27-3.36 (m, 4H), 6.65 (br s, 1H), 6.81-6.94 (m, 2H), 7.31 (br s, 1H), 7.56-7.63 (m, 1H), 9.02 (br s, 2H).

### Step 4

### Intermediate 814: 4-(9-(3-bromo-2-formylphenyl)-3,9-diazaspiro[5.5]undecan-3-yl)-2-fluorobenzamide

To a solution of 2-fluoro-4-(3,9-diazaspiro[5.5]undecan-3-yl)benzamide hydrochloride (Intermediate 813, 15.0 g, 51.5 mmol) in dimethylformamide (75 mL) were added potassium carbonate (24.9 g, 180 mmol) followed by 2-bromo-6-fluorobenzaldehyde (CAS No. 360575-28-6, 12.0 g, 56.6 mmol) and the reaction mixture was heated at 70° C. for 16 h. The reaction mixture was quenched with water (250 mL), the resultant precipitate was collected by filtration and dried in vacuo. The crude material was purified by combi-flash chromatography, by eluting with 70% ethyl acetate in heptane, to afford 4-(9-(3-bromo-2-formylphenyl)-3,9-diazaspiro[5.5]undecan-3-yl)-2-fluorobenzamide (Intermediate 814, 15.0 g, 62%) as a pale yellow solid. Mass spec m/z 476.1 [M+H+2]⁺.

### Step 5

### Intermediate 815: 4-(9-(3-bromo-2-(((2,6-dioxopiperidin-3-yl)amino)methyl)phenyl)-3,9-diazaspiro[5.5]undecan-3-yl)-2-fluorobenzamide

To a solution of 4-(9-(3-bromo-2-formylphenyl)-3,9-diazaspiro[5.5]undecan-3-yl)-2-fluorobenzamide (Intermediate 814, 5.0 g, 10.5 mmol) in acetonitrile (50 mL) was added 3-aminopiperidine-2,6-dione hydrochloride (CAS No. 24666-56-6, 1.96 g, 11.6 mmol) followed by triethylamine (4.43 mL, 31.6 mmol) and the reaction mixture was stirred at room temperature for 1 h. Sodium cyanoborohydride (2.09 g, 31.6 mmol) was then added and the reaction mixture was heated at 70° C. for 16 h. The reaction mixture was quenched with water (100 mL) and extracted with ethyl acetate (3×100 mL). The combined organic layers were dried over anhydrous Na₂SO₄ filtered, and concentrated in vacuo. The crude material was purified by combi-flash chromatography, by eluting with 90% ethyl acetate in heptane, to afford 4-(9-(3-bromo-2-(((2,6-dioxopiperidin-3-yl)amino)methyl)phenyl)-3,9-diazaspiro[5.5]undecan-3-yl)-2-fluorobenzamide (Intermediate 815, 1.40 g, 23%) as an off-white solid. ¹H NMR (400 MHz, DMSO-d₆) δ ppm 1.53-1.75 (m, 9H), 2.25-2.32 (m, 2H), 2.51-2.58 (m, 4H), 2.78-2.84 (m, 2H), 2.94-3.10 (m, 3H), 3.23-3.31 (m, 2H), 3.86-4.00 (m, 2H), 6.69-6.80 (m, 2H), 7.14-7.34 (m, 5H), 7.61 (t, J=9.20 Hz, 1H), 10.78 (s, 1H). Mass spec m/z 588.2 [M+H]⁺.

### Step 6

### Intermediate 816: 4-(9-(2-(2,6-dioxopiperidin-3-yl)-1-oxoisoindolin-4-yl)-3,9-diazaspiro[5.5]undecan-3-yl)-2-fluorobenzamide

To a solution of 4-(9-(3-bromo-2-(((2,6-dioxopiperidin-3-yl)amino)methyl)phenyl)-3,9-diazaspiro[5.5]undecan-3-yl)-2-fluorobenzamide (Intermediate 815, 3.0 g, 5.11 mmol) in 1,4-dioxane (30 mL) was added triethylamine (2.15 mL, 15.4 mmol). The reaction mixture was purged with argon for 15 min then Xantphos (0.61 g, 1.02 mmol) followed by PdCl₂(dppf) (0.78 g, 1.02 mmol) and tungsten hexacarbonyl (0.90 g, 2.55 mmol) were added. The reaction mixture was further purged with argon for 10 min and heated at 100° C. for 16 h. The reaction mixture was filtered through a celite bed and the filter pad was washed with ethyl acetate (200 mL). The filtrate was concentrated in vacuo and the crude material was purified by combi-flash chromatography, by eluting with 100% ethyl acetate, to afford 4-(9-(2-(2,6-dioxopiperidin-3-yl)-1-oxoisoindolin-4-yl)-3,9-diazaspiro[5.5]undecan-3-yl)-2-fluorobenzamide (Intermediate 816, 2.0 g, 73%) as a white solid. ¹H NMR (400 MHz, DMSO-d₆) δ ppm 1.52-1.65 (m, 8H), 2.41-2.49 (m, 3H), 2.51-2.67 (m, 4H), 2.86-2.96 (m, 1H), 3.00-3.14 (m, 4H), 4.21-4.31 (m, 1H), 4.42-4.46 (m, 1H), 5.09-5.14 (m, 1H), 6.67-6.80 (m, 2H), 7.16-7.20 (m, 2H), 7.28-7.30 (m, 2H), 7.41-7.45 (m, 1H), 7.58-7.63 (m, 1H), 10.98 (s, 1H). Mass spec m/z 534.3 [M+H]⁺.

### Step 7

### Intermediate 817: tert-butyl 6-(4-(9-(2-(2,6-dioxopiperidin-3-yl)-1-oxoisoindolin-4-yl)-3,9-diazaspiro[5.5]undecan-3-yl)-2-fluorobenzamido)-2-((R)-1-methylpyrrolidin-2-yl)-1H-pyrrolo[3,2-c]pyridine-1-carboxylate

To a solution of 4-(9-(2-(2,6-dioxopiperidin-3-yl)-1-oxoisoindolin-4-yl)-3,9-diazaspiro[5.5]undecan-3-yl)-2-fluorobenzamide (Intermediate 816, 0.4 g, 0.74 mmol) in 1,4-dioxane (2 mL) was added tert-butyl (R)-6-bromo-2-(1-methylpyrrolidin-2-yl)-1H-pyrrolo[3,2-c]pyridine-1-carboxylate (Intermediate 4, 0.34 g, 0.89 mmol) followed by cesium carbonate (0.73 g, 2.24 mmol). The reaction mixture was purged with argon for 15 min then EPhos Pd G4 (0.14 g, 0.14 mmol) and EPhos (0.063 g, 0.11 mmol) were added. The reaction mixture further was purged with argon for 10 min and heated at 110° C. for 3 h. The reaction mixture was filtered through a celite bed, the filter pad was washed with ethyl acetate (100 mL) and the filtrate concentrated in vacuo. The crude material was purified by combi-flash chromatography, by eluting with 5% MeOH in DCM, to afford tert-butyl 6-(4-(9-(2-(2,6-dioxopiperidin-3-yl)-1-oxoisoindolin-4-yl)-3,9-diazaspiro[5.5]undecan-3-yl)-2-fluorobenzamido)-2-((R)-1-methylpyrrolidin-2-yl)-1H-pyrrolo[3,2-c]pyridine-1-carboxylate (Intermediate 817, 0.2 g, 32%) as an off-white solid. ¹H NMR (400 MHz, DMSO-d₆) δ ppm 1.54-1.75 (m, 20H), 2.31-2.38 (m, 5H), 2.52-2.56 (m, 2H), 2.84-2.93 (m, 1H), 3.01-3.18 (m, 6H), 3.48-3.52 (m, 4H), 3.87-3.91 (m, 1H), 4.28-4.32 (m, 1H), 4.42-4.47 (m, 1H), 5.12-5.15 (m, 1H), 6.73-6.87 (m, 2H), 7.18-7.21 (m, 1H), 7.28-7.31 (m, 2H), 7.41-7.45 (m, 1H), 7.69-7.74 (m, 1H), 8.54 (s, 1H), 8.93 (s, 1H), 9.75 (d, J=4.80 Hz, 1H), 10.98 (s, 1H). Mass spec m/z 833.6 [M+H]⁺.

### Step 8

### Example 202: 4-(9-(2-(2,6-dioxopiperidin-3-yl)-1-oxoisoindolin-4-yl)-3,9-diazaspiro[5.5]undecan-3-yl)-2-fluoro-N-(2-((R)-1-methylpyrrolidin-2-yl)-1H-pyrrolo[3,2-c]pyridin-6-yl)benzamide

To a solution of tert-butyl 6-(4-(9-(2-(2,6-dioxopiperidin-3-yl)-1-oxoisoindolin-4-yl)-3,9-diazaspiro[5.5]undecan-3-yl)-2-fluorobenzamido)-2-((R)-1-methylpyrrolidin-2-yl)-1H-pyrrolo[3,2-c]pyridine-1-carboxylate (Intermediate 817, 0.2 g, 0.24 mmol) in dichloromethane (2 mL) was added 4M hydrochloric acid in 1,4-dioxane (2.0 mL). The reaction mixture was stirred at room temperature for 2 h. The reaction mixture was then concentrated in vacuo and the crude material purified by preparative HPLC (Method A) to afford 4-(9-(2-(2,6-dioxopiperidin-3-yl)-1-oxoisoindolin-4-yl)-3,9-diazaspiro[5.5]undecan-3-yl)-2-fluoro-N-(2-((R)-1-methylpyrrolidin-2-yl)-1H-pyrrolo[3,2-c]pyridin-6-yl)benzamide (Example 202, 0.05 g, 30%) as an off-white solid. ¹H NMR (400 MHz, DMSO-d₆) δ ppm 1.58-1.67 (m, 8H), 1.74-2.01 (m, 5H), 2.12-2.18 (m, 4H), 2.21-2.28 (m, 1H), 2.40-2.47 (m, 1H), 2.52-2.60 (m, 1H), 2.87-2.97 (m, 1H), 3.04-3.40 (m, 5H), 3.35-3.45 (m, 4H), 4.28-4.32 (m, 1H), 4.42-4.47 (m, 1H), 5.10-5.15 (m, 1H), 6.38 (s, 1H), 6.80-6.89 (m, 2H), 7.20 (d, J=7.60 Hz, 1H), 7.30 (d, J=7.20 Hz, 1H), 7.43 (t, J=7.60 Hz, 1H), 7.74 (t, J=9.20 Hz, 1H), 8.20 (s, 1H), 8.44 (s, 1H), 9.49 (d, J=6.00 Hz, 1H), 10.99 (s, 1H), 11.37 (s, 1H). Mass spec m/z 733.3 [M+H]⁺.

### Example 203 was Prepared Following Scheme 197

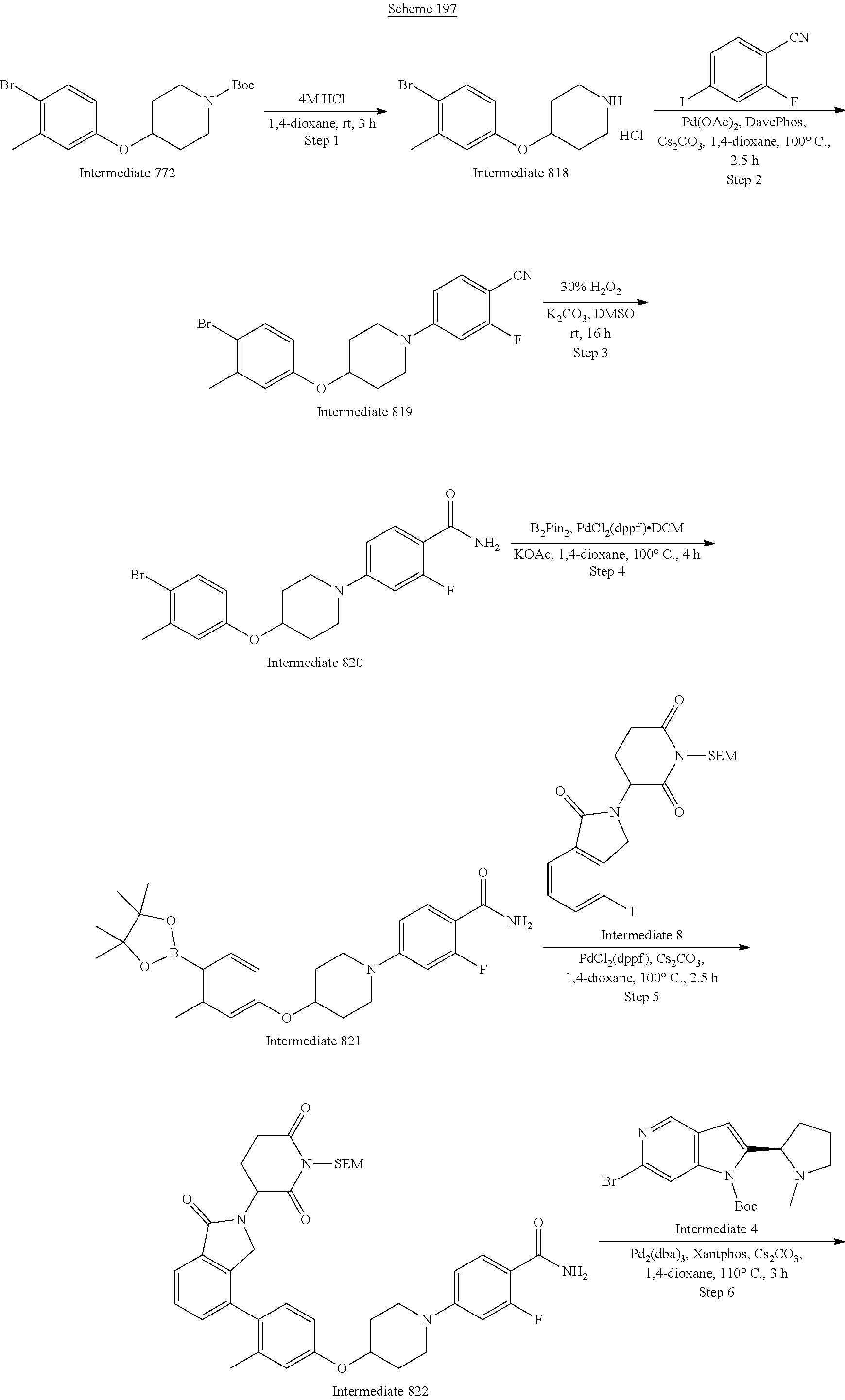

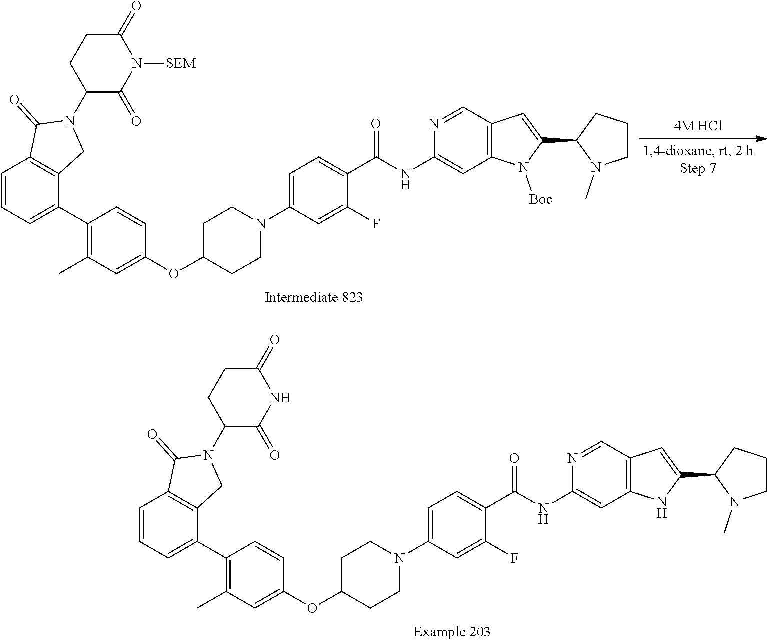 [SMILES: Cc1cc(OC2CCN(c3ccc(C(=O)Nc4cc5c(cn4)cc([C@H]4CCCN4C)n5C(=O)OC(C)(C)C)c(F)c3)CC2)ccc1-c1cccc2c1CN(C1CCC(=O)N(COCC[Si](C)(C)C)C1=O)C2=O ; Cc1cc(OC2CCN(c3ccc(C(=O)Nc4cc5[nH]c([C@H]6CCCN6C)cc5cn4)c(F)c3)CC2)ccc1-c1cccc2c1CN(C1CCC(=O)NC1=O)C2=O]

### Step 1

### Intermediate 818: 4-(4-bromo-3-methylphenoxy)piperidine hydrochloride

To a solution of tert-butyl 4-(4-bromo-3-methylphenoxy)piperidine-1-carboxylate (Intermediate 772, 6.0 g, 16.2 mmol) in dichloromethane (60 mL) was added 4M hydrochloric acid in dioxane (60 mL). The reaction mixture was stirred at room temperature for 3 h then concentrated in vacuo to afford 4-(4-bromo-3-methylphenoxy)piperidine hydrochloride (Intermediate 818, 4.5 g, 91%) as an off-white solid, which was used for the next step without further purification. ¹H NMR (400 MHz, DMSO-d₆) δ ppm 1.76-1.83 (m, 2H), 2.04-2.08 (m, 2H), 2.29 (s, 3H), 3.01-3.08 (m, 2H), 3.17-3.22 (m, 2H), 4.23-4.28 (m, 1H), 6.78-6.81 (m, 1H), 7.03 (s, 1H), 7.44-7.47 (m, 1H), 8.88 (br s, 2H).

### Step 2

### Intermediate 819: 4-(4-(4-bromo-3-methylphenoxy)piperidin-1-yl)-2-fluorobenzonitrile

To a solution of 4-(4-bromo-3-methylphenoxy)piperidine hydrochloride (Intermediate 818, 5.0 g, 16.30 mmol) in 1,4-dioxane (50 mL) was added tert-butyl 4-bromo-2-fluorobenzoate (CAS No. 889858-12-2, 4.83 g, 19.6 mmol) followed by cesium carbonate (15.9 g, 48.9 mmol). The reaction mixture was purged with argon for 15 min then Pd(OAc)₂ (0.57 g, 2.44 mmol) followed by DavePhos (1.01 g, 2.44 mmol) were added. The reaction mixture was further purged with argon for 10 min and heated at 100° C. for 2.5 h. The reaction mixture was then concentrated in vacuo. The crude material was purified by combi-flash chromatography, by eluting with 7% MeOH in DCM, to afford 4-(4-(4-bromo-3-methylphenoxy)piperidin-1-yl)-2-fluorobenzonitrile (Intermediate 819, 4.5 g, 71%) as an off-white solid. ¹H NMR (400 MHz, DMSO-d₆) δ ppm 1.57-1.64 (m, 2H), 1.94-1.96 (m, 2H), 2.28 (s, 3H), 3.29-3.34 (m, 2H), 3.68-3.72 (m, 2H), 4.59-4.63 (m, 1H), 6.75-6.86 (m, 2H), 6.93 (s, 1H), 6.96-7.00 (m, 1H), 7.40-7.43 (m, 1H), 7.53-7.57 (m, 1H). Mass spec m/z 391.0 [M+H+2]⁺.

### Step 4

### Intermediate 820: 4-(4-(4-bromo-3-methylphenoxy)piperidin-1-yl)-2-fluorobenzamide

To a cooled (0° C.) solution of 4-(4-(4-bromo-3-methylphenoxy)piperidin-1-yl)-2-fluorobenzonitrile (Intermediate 819, 4.5 g, 12 mmol) in dimethyl sulfoxide (45 mL) were added potassium carbonate (4.8 g, 35 mmol) followed by 30% hydrogen peroxide (3.5 mL, 120 mmol) and the reaction mixture was stirred at room temperature for 16 h. The reaction mixture was quenched with ice cold water (300 mL). The resultant precipitate was collected by filtration and dried in vacuo to afford 4-(4-(4-bromo-3-methylphenoxy)piperidin-1-yl)-2-fluorobenzamide (Intermediate 820, 4.0 g, 85%) as an off-white solid, which was used for the next step without further purification. Mass spec m/z 409.0 [M+H+2]⁺.

### Step 4

### Intermediate 821: 2-fluoro-4-(4-(3-methyl-4-(4,4,5,5-tetramethyl-1,3,2-dioxaborolan-2-yl)phenoxy)piperidin-1-yl)benzamide

To a solution of 4-(4-(4-bromo-3-methylphenoxy)piperidin-1-yl)-2-fluorobenzamide (Intermediate 820, 2.5 g, 6.1 mmol) 1,4-dioxane (15 mL) was added 4,4,4′,4′,5,5,5′,5′-octamethyl-2,2′-bi-1,3,2-dioxaborolane (1.9 g, 7.4 mmol) followed by potassium acetate (1.8 g, 18.0 mmol). The reaction mixture was purged with argon for 15 min then PdCl₂(dppf) CH₂Cl₂ (0.66 g, 0.80 mmol) was added. The reaction mixture was further purged with argon for 10 min and heated at 100° C. for 4 h. The reaction mixture was filtered through a celite bed and the filter pad was washed with ethyl acetate (100 mL). The filtrate was concentrated in vacuo to afford 2-fluoro-4-(4-(3-methyl-4-(4,4,5,5-tetramethyl-1,3,2-dioxaborolan-2-yl)phenoxy)piperidin-1-yl)benzamide (Intermediate 821, 2.5 g, 72%) as an off-white solid. ¹H NMR (400 MHz, DMSO-d₆) δ ppm 1.25 (s, 12H), 1.59-1.65 (m, 2H), 1.92-1.98 (m, 2H), 2.24-2.28 (m, 1H), 2.40 (s, 3H), 3.19-3.23 (m, 2H), 3.61-3.64 (m, 2H), 6.71-6.79 (m, 4H), 7.13 (br s, 1H), 7.27 (br s, 1H), 7.53-7.61 (m, 2H). Mass spec m/z 455.1 [M+H]⁺.

### Step 5

### Intermediate 822: 4-(4-(4-(2-(2,6-dioxo-1-((2-(trimethylsilyl)ethoxy)methyl)piperidin-3-yl)-1-oxoisoindolin-4-yl)-3-methylphenoxy)piperidin-1-yl)-2-fluorobenzamide

To a solution of 2-fluoro-4-(4-(3-methyl-4-(4,4,5,5-tetramethyl-1,3,2-dioxaborolan-2-yl)phenoxy)piperidin-1-yl)benzamide (Intermediate 821, 2.0 g, 4.40 mmol) in 1,4-dioxane (25 mL) was added 3-(4-iodo-1-oxoisoindolin-2-yl)-1-((2-(trimethylsilyl)ethoxy)methyl)piperidine-2,6-dione (Intermediate 8, 2.64 g, 5.28 mmol) followed by cesium carbonate (4.53 g, 13.2 mmol). The reaction mixture was purged with argon for 15 min then PdCl₂(dppf) (0.71 g, 0.88 mmol) was added. The reaction mixture was further purged with argon for 10 min and heated at 100° C. for 2.5 h. The reaction mixture was concentrated in vacuo. The crude material was purified by combi-flash chromatography, by eluting with 7% MeOH in DCM, to afford 4-(4-(4-(2-(2,6-dioxo-1-((2-(trimethylsilyl)ethoxy)methyl)piperidin-3-yl)-1-oxoisoindolin-4-yl)-3-methylphenoxy)piperidin-1-yl)-2-fluorobenzamide (Intermediate 822, 1.6 g, 32%) as an off-white solid. ¹H NMR (400 MHz, DMSO-d₆) δ ppm −0.09 (s, 9H), 0.70-0.86 (m, 2H), 1.60-1.77 (m, 2H), 1.99-2.14 (m, 8H), 2.03-2.05 (m, 2H), 2.07-2.15 (m, 2H), 3.47 (s, 3H), 3.61-3.73 (m, 2H), 4.34-4.54 (m, 2H), 4.63-4.83 (m, 2H), 6.73-6.86 (m, 2H), 6.91 (d, J=8.51 Hz, 1H), 6.99 (s, 1H), 7.11-7.23 (m, 2H), 7.31 (br s, 1H), 7.39-7.51 (m, 1H), 7.52-7.67 (m, 2H), 7.67-7.77 (m, 1H).

### Step 6

### Intermediate 823: tert-butyl 6-(4-(4-(4-(2-(2,6-dioxo-1-((2-(trimethylsilyl)ethoxy)methyl)piperidin-3-yl)-1-oxoisoindolin-4-yl)-3-methylphenoxy)piperidin-1-yl)-2-fluorobenzamido)-2-((R)-1-methylpyrrolidin-2-yl)-1H-pyrrolo[3,2-c]pyridine-1-carboxylate

To a solution of 4-(4-(4-(2-(2,6-dioxo-1-((2-(trimethylsilyl)ethoxy)methyl)piperidin-3-yl)-1-oxoisoindolin-4-yl)-3-methylphenoxy)piperidin-1-yl)-2-fluorobenzamide (Intermediate 822, 0.50 g, 0.70 mmol) in 1,4-dioxane (5 mL) was added tert-butyl (R)-6-bromo-2-(1-methylpyrrolidin-2-yl)-1H-pyrrolo[3,2-c]pyridine-1-carboxylate (Intermediate 4, 0.325 g, 0.85 mmol) followed by cesium carbonate (0.40 g, 2.10 mmol). The reaction mixture was purged with argon for 15 min then Pd₂(dba)₃ (0.17 g, 0.18 mmol) followed by Xantphos (0.17 g, 0.29 mmol) were added. The reaction mixture was further purged with argon for 10 min and heated at 110° C. for 3 h. The reaction mixture was filtered through a celite bed, the filter pad was washed with dichloromethane (100 mL) and the filtrate concentrated in vacuo. The crude material was purified by combi-flash chromatography, by eluting with 90% ethyl acetate in heptane, to afford tert-butyl 6-(4-(4-(4-(2-(2,6-dioxo-1-((2-(trimethylsilyl)ethoxy)methyl)piperidin-3-yl)-1-oxoisoindolin-4-yl)-3-methylphenoxy)piperidin-1-yl)-2-fluorobenzamido)-2-((R)-1-methylpyrrolidin-2-yl)-1H-pyrrolo[3,2-c]pyridine-1-carboxylate (Intermediate 823, 0.36 g, 51%) as an off-white solid.

Mass spec m/z 1000.2 [M+H]⁺.

### Step 7

### Example 203: 4-(4-(4-(2-(2,6-dioxopiperidin-3-yl)-1-oxoisoindolin-4-yl)-3-methylphenoxy)piperidin-1-yl)-2-fluoro-N-(2-((R)-1-methylpyrrolidin-2-yl)-1H-pyrrolo[3,2-c]pyridin-6-yl)benzamide

To a solution of tert-butyl 6-(4-(4-(4-(2-(2,6-dioxopiperidin-3-yl)-1-oxoisoindolin-4-yl)-3-methylphenoxy)piperidin-1-yl)-2-fluorobenzamido)-2-((R)-1-methylpyrrolidin-2-yl)-1H-pyrrolo[3,2-c]pyridine-1-carboxylate (Intermediate 823, 0.36 g, 0.35 mmol) in acetonitrile (15 mL) was added methanesulfonic acid (0.23 mL, 3.59 mmol) and the reaction mixture was heated at 60° C. for 2 h. The reaction mixture was cooled to room temperature, then N,N′-dimethylethylenediamine (0.2 mL, 1.79 mmol) followed by triethylamine (1.01 mL, 7.19 mmol) were added and the mixture was stirred at room temperature for 2 h. The reaction mixture was concentrated in vacuo and the resultant residue was diluted with water (50 mL). The resultant precipitate was collected by filtration and dried in vacuo. The crude solid was purified by preparative HPLC (Method A) to afford 4-(4-(4-(2-(2,6-dioxopiperidin-3-yl)-1-oxoisoindolin-4-yl)-3-methylphenoxy)piperidin-1-yl)-2-fluoro-N-(2-((R)-1-methylpyrrolidin-2-yl)-1H-pyrrolo[3,2-c]pyridin-6-yl)benzamide (Example 203, 0.02 g, 7%) as an off-white solid. ¹H NMR (400 MHz, DMSO-d₆) δ ppm 1.71-1.96 (m, 8H), 2.06-2.015 (m, 7H), 2.16 (s, 3H), 2.22-2.27 (m, 1H), 2.32-2.40 (m, 1H), 2.84-2.87 (m, 1H), 3.11-3.15 (m, 1H), 3.30-3.35 (m, 1H), 3.72-3.78 (m, 2H), 4.06-4.10 (m, 1H), 4.19-4.23 (m, 1H), 4.65-4.70 (m, 1H), 5.09-5.14 (m, 1H), 6.38 (s, 1H), 6.86-6.92 (m, 3H), 6.99 (s, 1H), 7.17 (d, J=8.40 Hz, 1H), 7.38-7.48 (m, 1H), 7.57-7.61 (m, 1H), 7.73-7.77 (m, 2H), 8.20 (s, 1H), 8.45 (s, 1H), 9.56 (br s, 1H), 10.93 (br s, 1H), 11.37 (br s, 1H). Mass spec m/z 770.3 [M+H]⁺.

### Example 204 was Prepared Following Scheme 198

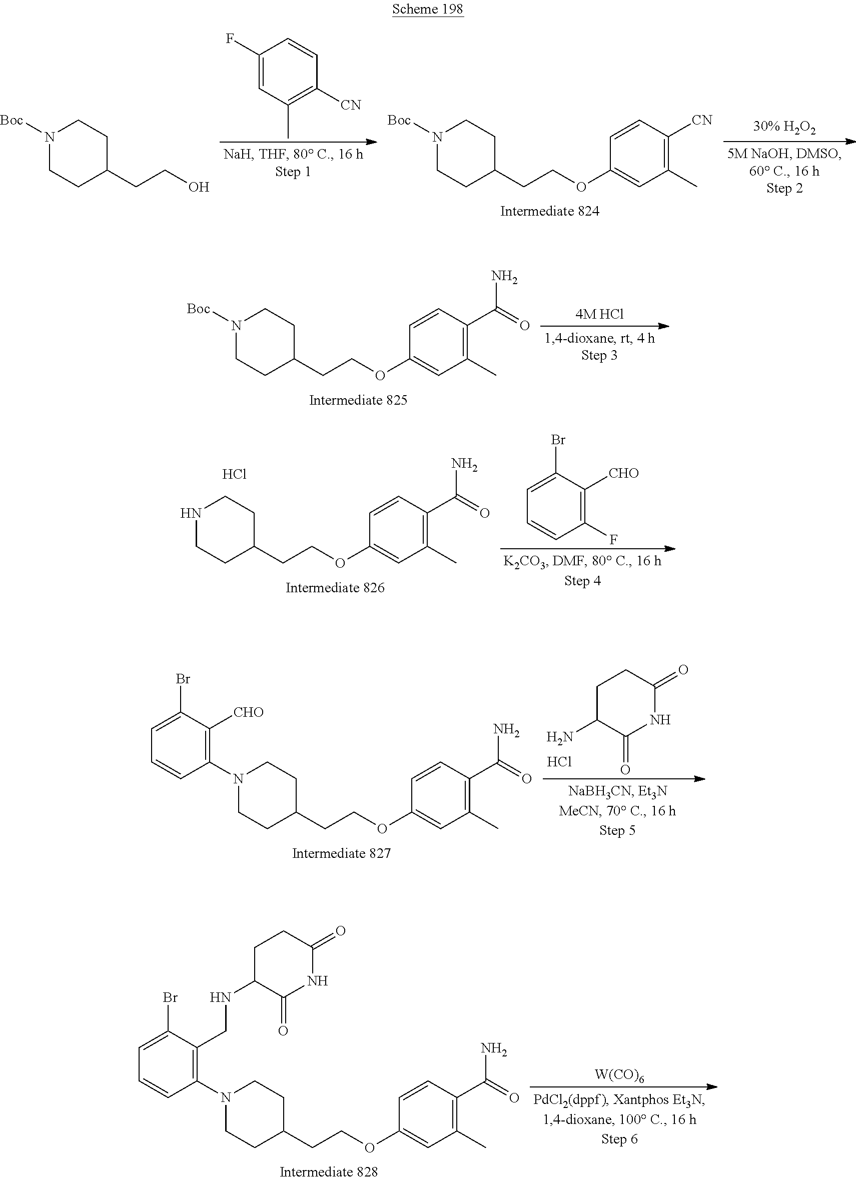

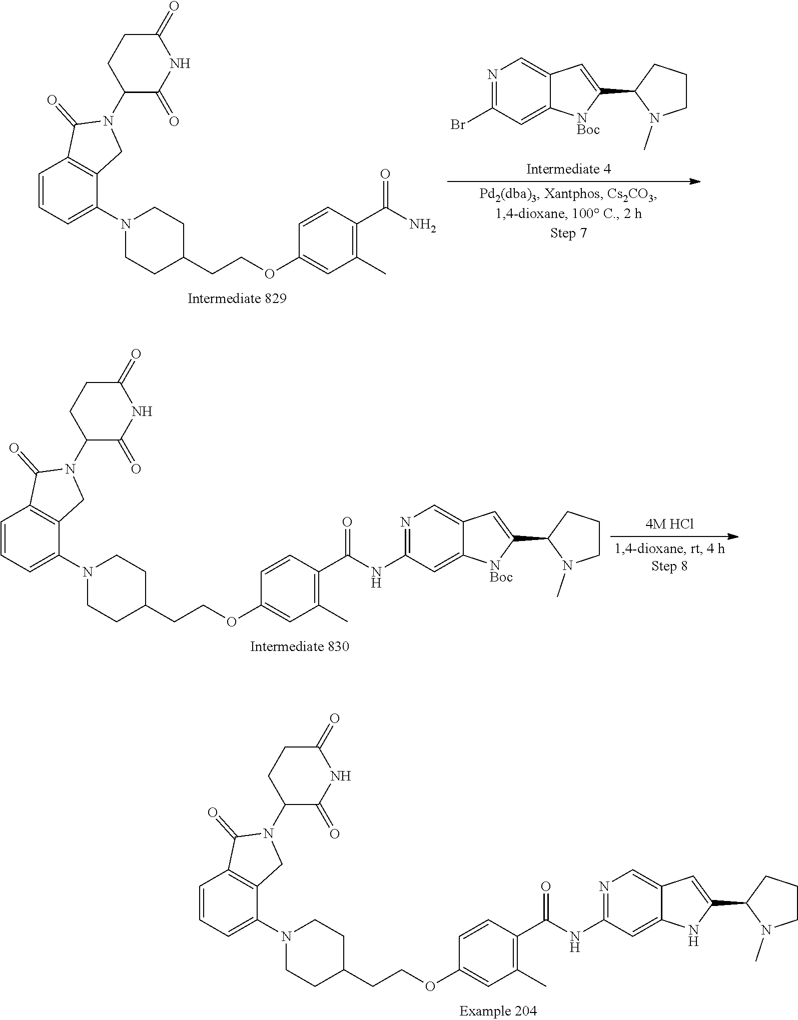 [SMILES: Cc1cc(OCCC2CCN(c3cccc4c3CN(C3CCC(=O)NC3=O)C4=O)CC2)ccc1C(N)=O ; CN1CCC[C@@H]1c1cc2cnc(Br)cc2n1C(=O)OC(C)(C)C ; Cc1cc(OCCC2CCN(c3cccc4c3CN(C3CCC(=O)NC3=O)C4=O)CC2)ccc1C(=O)Nc1cc2c(cn1)cc([C@H]1CCCN1C)n2C(=O)OC(C)(C)C ; Cc1cc(OCCC2CCN(c3cccc4c3CN(C3CCC(=O)NC3=O)C4=O)CC2)ccc1C(=O)Nc1cc2[nH]c([C@H]3CCCN3C)cc2cn1]

### Step 1

### Intermediate 824: tert-butyl 4-(2-(4-cyano-3-methylphenoxy)ethyl)piperidine-1-carboxylate

To a cooled (0° C.) solution of 4-fluoro-2-methylbenzonitrile (CAS No. 147754-12-9, 15.3 g, 113.4 mmol) in tetrahydrofuran (200 mL) was added sodium hydride (60% in mineral oil, 3.48 g, 131 mmol) and the reaction mixture was stirred at 0° C. for 30 min tert-Butyl 4-(2-hydroxyethyl)piperidine-1-carboxylate (CAS No. 89151-44-0, 20 g, 87.2 mmol) was then added and the reaction mixture was stirred at 80° C. for 16 h. After completion, the reaction mixture was quenched with ice cold water (200 mL) and extracted with ethyl acetate (2×200 mL). The combined organic layers were dried over Na₂SO₄, filtered and concentrated in vacuo. The crude material was purified by combi-flash chromatography, by eluting with 30% ethyl acetate in heptane, to afford tert-butyl 4-(2-(4-cyano-3-methylphenoxy)ethyl)piperidine-1-carboxylate (Intermediate 824, 25 g, 83%) as an off-white solid. ¹H NMR (400 MHz, DMSO-d₆) δ ppm 1.19-1.25 (m, 3H), 1.45 (s, 9H), 1.68-1.75 (m, 4H), 2.50 (s, 3H), 2.66-2.72 (m, 2H), 4.01-4.05 (m, 4H), 6.77 (d, J=8.40 Hz, 2H), 7.50 (d, J=8.40 Hz, 1H).

### Step 2

### Intermediate 825: tert-butyl 4-(2-(4-carbamoyl-3-methylphenoxy)ethyl)piperidine-1-carboxylate

To a cooled (0° C.) solution of tert-butyl 4-(2-(4-cyano-3-methylphenoxy)ethyl)piperidine-1-carboxylate (Intermediate 824, 20 g, 58.1 mmol) in dimethyl sulfoxide (100 mL) was added 5M NaOH (25 mL, 384 mmol) followed by 30% hydrogen peroxide (60 mL) and the reaction mixture was stirred at 60° C. for 16 h. The reaction mixture was then quenched with ice cold water (200 mL) and extracted with ethyl acetate (2×200 mL). The combined organic layers were dried over Na₂SO₄, filtered and concentrated in vacuo to afford tert-butyl 4-(2-(4-carbamoyl-3-methylphenoxy)ethyl)piperidine-1-carboxylate (Intermediate 825, 20 g, 95%) as an off-white solid, which was used for the next step without further purification. ¹H NMR (400 MHz, DMSO-d₆) δ ppm 1.02-1.10 (m, 2H), 1.38 (s, 9H), 1.65-1.72 (m, 5H), 2.36 (s, 3H), 2.52-2.57 (m, 2H), 3.89-3.94 (m, 2H), 4.00-4.05 (m, 2H), 6.73-6.77 (m, 2H), 7.13 (br s, 1H), 7.34 (d, J=8.61 Hz, 1H), 7.53 (br s, 1H). Mass spec m/z 363.1 [M+H]⁺.

### Step 3

### Intermediate 826: 2-methyl-4-(2-(piperidin-4-yl)ethoxy)benzamide hydrochloride

To a solution of tert-butyl 4-(2-(4-carbamoyl-3-methylphenoxy)ethyl)piperidine-1-carboxylate (Intermediate 825, 15 g, 41.38 mmol) in dichloromethane (20 mL) was added 4M hydrochloric acid in 1,4-dioxane (50 mL). The reaction mixture was stirred at room temperature for 4 h. The reaction mixture was then concentrated in vacuo to afford 2-methyl-4-(2-(piperidin-4-yl)ethoxy)benzamide hydrochloride (Intermediate 826, 10 g, 92%) as an off-white solid, which was used for the next step without further purification. Mass spec m/z 263.1 [M+H]⁺.

### Step 4

### Intermediate 827: 4-(2-(1-(3-bromo-2-formylphenyl)piperidin-4-yl)ethoxy)-2-methylbenzamide

To a solution of 2-methyl-4-(2-(piperidin-4-yl)ethoxy)benzamide hydrochloride (Intermediate 826, 7.0 g, 25.1 mmol) in dimethylformamide (20 mL) was added potassium carbonate (13.9 g, 101 mmol) followed by 2-bromo-6-fluorobenzaldehyde (CAS No. 360575-28-6, 6.12 g, 30.1 mmol) and the reaction mixture was heated to 80° C. for 16 h. The reaction mixture was quenched with water (250 mL) and extracted with ethyl acetate (2×125 mL). The combined organic layers were dried over Na₂SO₄, filtered and concentrated in vacuo to afford 4-(2-(1-(3-bromo-2-formylphenyl)piperidin-4-yl)ethoxy)-2-methylbenzamide (Intermediate 827, 10 g, 89%) as a pale yellow solid which was used for the next step without further purification. ¹H NMR (400 MHz, DMSO-d₆) δ ppm 1.40-1.45 (m, 2H), 1.59-1.65 (m, 1H), 1.71-1.84 (m, 4H), 2.73 (s, 3H), 2.82-2.89 (m, 2H), 3.15-3.20 (m, 2H), 4.04-4.12 (m, 2H), 7.13 (br s, 1H), 7.20-7.25 (m, 1H), 7.30-7.41 (m, 3H), 7.53 (s, 2H), 7.91 (br s, 1H), 10.38 (br s, 1H). Mass spec m/z 445.1 [M+H]⁺.

### Step 5

### Intermediate 828: 4-(2-(1-(3-bromo-2-(((2,6-dioxopiperidin-3-yl)amino)methyl)phenyl)piperidin-4-yl)ethoxy)-2-methylbenzamide

To a solution of 4-(2-(1-(3-bromo-2-formylphenyl)piperidin-4-yl)ethoxy)-2-methylbenzamide (Intermediate 827, 8.0 g, 18.0 mmol) in acetonitrile (50 mL) was added and 3-aminopiperidine-2,6-dione hydrochloride (CAS No. 24666-56-6, 3.54 g, 21.6 mmol) followed by triethylamine (7.5 mL, 53.9 mmol) and the reaction mixture was stirred at room temperature for 1 h. Sodium cyanoborohydride (3.56 g, 53.9 mmol) was then added and reaction mixture was heated at 70° C. for 16 h. The reaction mixture was quenched with water (100 mL) and extracted with ethyl acetate (3×100 mL). The combined organic layers were dried over Na₂SO₄, filtered and concentrated in vacuo. The crude material was purified by combi-flash chromatography, by eluting with 10% MeOH in DCM, to afford 4-(2-(1-(3-bromo-2-(((2,6-dioxopiperidin-3-yl)amino)methyl)phenyl)piperidin-4-yl)ethoxy)-2-methylbenzamide (Intermediate 828, 4.0 g, 40%) as a green semi-solid. ¹H NMR (400 MHz, DMSO-d₆) δ ppm 1.21-1.44 (m, 3H), 1.63-1.83 (m, 6H), 2.27-2.33 (m, 1H), 2.35 (s, 3H), 2.53-2.60 (m, 2H), 2.71-2.77 (m, 1H), 2.90-2.95 (m, 1H), 3.05-3.11 (m, 1H), 3.20-3.32 (m, 2H), 3.85-3.96 (m, 1H), 4.03-4.06 (m, 2H), 6.74-6.77 (m, 2H), 7.12-7.18 (m, 3H), 7.30-7.35 (m, 2H), 7.51 (br s, 2H), 10.76 (br s, 1H). Mass spec m/z 557.0 [M+H]⁺.

### Step 6

### Intermediate 829: 4-(2-(1-(2-(2,6-dioxopiperidin-3-yl)-1-oxoisoindolin-4-yl)piperidin-4-yl)ethoxy)-2-methylbenzamide

To a solution of 4-(2-(1-(3-bromo-2-(((2,6-dioxopiperidin-3-yl)amino)methyl)phenyl)piperidin-4-yl)ethoxy)-2-methylbenzamide (Intermediate 828, 3.5 g, 6.3 mmol) in 1,4-dioxane (35 mL) was added triethylamine (2.6 mL, 19 mmol). The reaction mixture was purged with argon gas for 15 min then Xantphos (0.75 g, 1.3 mmol) followed by PdCl₂(dppf) (0.92 g, 1.3 mmol) and tungsten hexacarbonyl (1.1 g, 3.1 mmol) were added. The reaction mixture was further purged with argon gas for 10 min and heated at 100° C. for 16 h. The reaction mixture was then filtered through a celite bed and the filter pad was washed with ethyl acetate (200 mL). The filtrate was concentrated in vacuo and the crude material purified by combi-flash chromatography, by eluting with 10% MeOH in DCM, to afford 4-(2-(1-(2-(2,6-dioxopiperidin-3-yl)-1-oxoisoindolin-4-yl)piperidin-4-yl)ethoxy)-2-methylbenzamide (Intermediate 829, 2.0 g, 63%) as a brown solid. Mass spec m/z 505.3 [M+H]⁺.

### Step 7

### Intermediate 830: tert-butyl 6-(4-(2-(1-(2-(2,6-dioxopiperidin-3-yl)-1-oxoisoindolin-4-yl)piperidin-4-yl)ethoxy)-2-methylbenzamido)-2-((R)-1-methylpyrrolidin-2-yl)-1H-pyrrolo[3,2-c]pyridine-1-carboxylate

To a solution of 4-(2-(1-(2-(2,6-dioxopiperidin-3-yl)-1-oxoisoindolin-4-yl)piperidin-4-yl)ethoxy)-2-methylbenzamide (Intermediate 829, 0.8 g, 1.58 mmol) in 1,4-dioxane (10.0 mL) was added tert-butyl (R)-6-bromo-2-(1-methylpyrrolidin-2-yl)-1H-pyrrolo[3,2-c]pyridine-1-carboxylate (Intermediate 4, 0.66 g, 1.74 mmol) followed by cesium carbonate (1.55 g, 4.75 mmol). The reaction mixture was purged with argon for 15 min, then Pd₂(dba)₃ (0.37 g, 0.39 mmol) and Xantphos (0.37 g, 0.63 mmol) were added. The reaction mixture was purged with argon for another 10 min and stirred at 100° C. for 2 h. The reaction mixture was then filtered through a celite bed, the filter pad was washed with ethyl acetate (100 mL) and the filtrate concentrated in vacuo. The crude material was purified by combi-flash chromatography, by eluting with 9% MeOH in DCM, to afford tert-butyl 6-(4-(2-(1-(2-(2,6-dioxopiperidin-3-yl)-1-oxoisoindolin-4-yl)piperidin-4-yl)ethoxy)-2-methylbenzamido)-2-((R)-1-methylpyrrolidin-2-yl)-1H-pyrrolo[3,2-c]pyridine-1-carboxylate (Intermediate 830, 0.5 g, 39%) as an off-white solid. Mass spec m/z 804.5 [M+H]⁺.

### Step 8

### Example 204: 4-(2-(1-(2-(2,6-dioxopiperidin-3-yl)-1-oxoisoindolin-4-yl)piperidin-4-yl)ethoxy)-2-methyl-N-(2-((R)-1-methylpyrrolidin-2-yl)-1H-pyrrolo[3,2-c]pyridin-6-yl)benzamide

To a solution of tert-butyl 6-(4-(2-(1-(2-(2,6-dioxopiperidin-3-yl)-1-oxoisoindolin-4-yl)piperidin-4-yl)ethoxy)-2-methylbenzamido)-2-((R)-1-methylpyrrolidin-2-yl)-1H-pyrrolo[3,2-c]pyridine-1-carboxylate (Intermediate 830, 0.5 g, 0.62 mmol) in dichloromethane (5.0 mL) was added 4M hydrochloric acid in 1,4-dioxane (5.0 mL). The reaction mixture was stirred at room temperature for 4 h. The reaction mixture was then concentrated in vacuo and the crude material purified by preparative HPLC (Method A) to afford 4-(2-(1-(2-(2,6-dioxopiperidin-3-yl)-1-oxoisoindolin-4-yl)piperidin-4-yl)ethoxy)-2-methyl-N-(2-((R)-1-methylpyrrolidin-2-yl)-1H-pyrrolo[3,2-c]pyridin-6-yl)benzamide (Example 204, 0.054 g, 12%) as an off-white solid. ¹H NMR (400 MHz, DMSO-d₆) δ ppm 1.33-1.46 (m, 2H), 1.64-1.71 (m, 1H), 1.71-1.78 (m, 2H), 1.78-1.92 (m, 5H), 1.95-2.03 (m, 1H), 2.11-2.15 (m, 2H), 2.16 (s, 3H), 2.22-2.28 (m, 1H), 2.30-2.35 (m, 1H), 2.41-2.46 (m, 3H), 2.58-2.63 (m, 1H), 2.69-2.80 (m, 2H), 2.86-2.97 (m, 1H), 3.11-3.17 (m, 1H), 3.37-3.44 (m, 2H), 4.08-4.11 (m, 2H), 4.26-4.33 (m, 1H), 4.40-4.46 (m, 1H), 5.09-5.13 (m, 1H), 6.37 (s, 1H), 6.80-6.85 (m, 2H), 7.18 (d, J=8.00 Hz, 1H), 7.30 (d, J=7.13 Hz, 1H), 7.40-7.49 (m, 2H), 8.16 (s, 1H), 8.45 (s, 1H), 10.18 (br s, 1H), 10.97 (br s, 1H), 11.31 (br s, 1H). Mass spec m/z 704.3 [M+H]⁺.

### Example 205 was Prepared Following Scheme 198

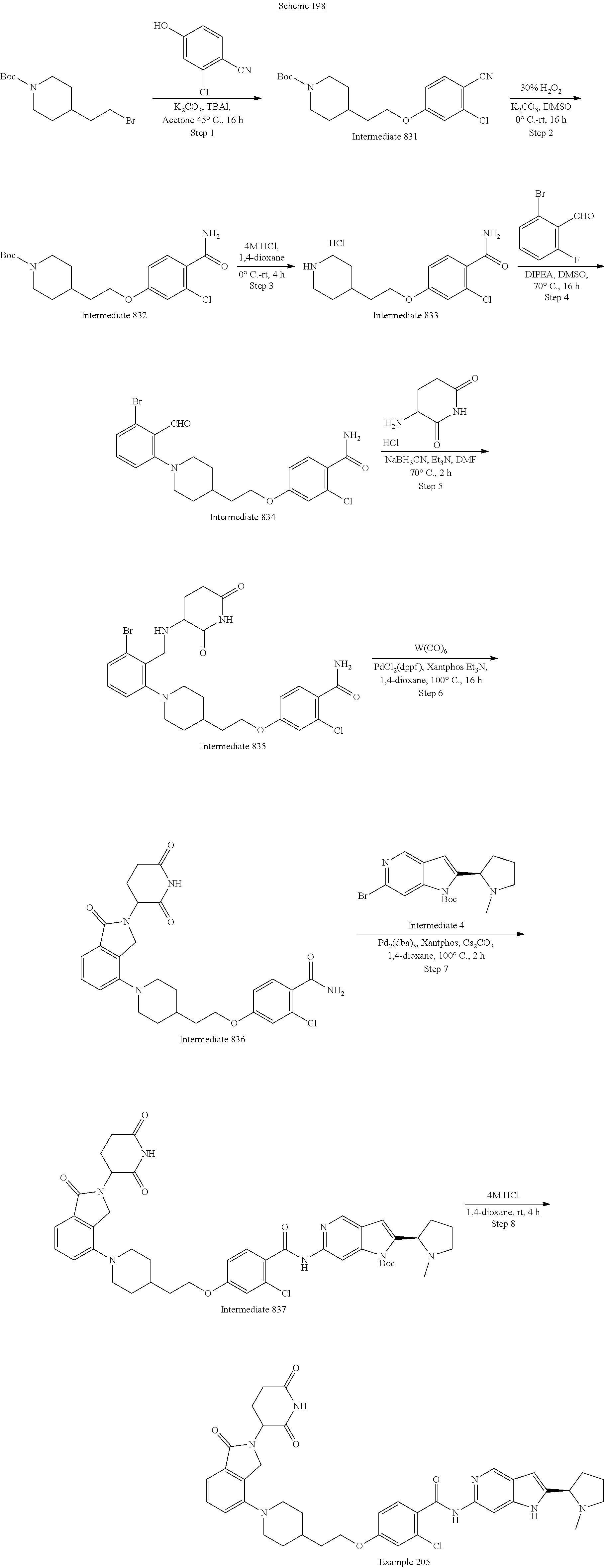

### Step 1

### Intermediate 831: tert-butyl 4-(2-(3-chloro-4-cyanophenoxy)ethyl)piperidine-1-carboxylate

To a solution of 2-chloro-4-hydroxybenzonitrile (CAS No. 3336-16-1, 5.25 g, 34.2 mmol) in acetone (100 mL) was added tert-butyl 4-(2-bromoethyl)piperidine-1-carboxylate (CAS No. 169457-73-2, 10 g, 34.2 mmol), potassium carbonate (18.91 g, 137 mmol) and tetrabutylammonium iodide (1.26 g 3.42 mmol). The reaction mixture was stirred at 45° C. for 16 h then concentrated in vacuo. The crude material was purified by combi-flash chromatography, by eluting with 30% ethyl acetate in heptane, to afford tert-butyl 4-(2-(3-chloro-4-cyanophenoxy)ethyl)piperidine-1-carboxylate (Intermediate 831, 10.0 g, 80%) as an off-white solid. ¹H NMR (400 MHz, DMSO-d₆) δ ppm 0.98-1.11 (m, 2H), 1.38 (s, 9H), 1.61-1.67 (m, 5H), 2.60-2.78 (m, 2H), 3.88-3.97 (m, 2H), 4.12-4.15 (m, 2H), 7.08-7.10 (m, 1H), 7.34 (s, 1H), 7.86 (d, J=8.80 Hz, 1H).

### Step 2

### Intermediate 832: tert-butyl 4-(2-(4-carbamoyl-3-chlorophenoxy)ethyl)piperidine-1-carboxylate

To a cooled (0° C.) solution of tert-butyl 4-(2-(3-chloro-4-cyanophenoxy)ethyl)piperidine-1-carboxylate (Intermediate 831, 10.0 g, 27.4 mmol) in dimethyl sulfoxide (100 mL) was added potassium carbonate (7.58 g, 54.8 mmol) followed by 30% hydrogen peroxide (9.32 mL, 82.3 mmol) and the reaction mixture was stirred at room temperature for 16 h. The reaction mixture was quenched with ice cold water (300 mL), the resultant precipitate was collected by filtration and dried in vacuo to afford tert-butyl 4-(2-(4-carbamoyl-3-chlorophenoxy)ethyl)piperidine-1-carboxylate (Intermediate 832, 9.0 g, 86%) as an off-white solid, which was used for the next step without further purification. ¹H NMR (400 MHz, DMSO-d₆) δ ppm 0.99-1.09 (m, 2H), 1.38 (s, 9H), 1.62-1.70 (m, 5H), 2.61-2.76 (m, 2H), 3.88-3.97 (m, 2H), 4.04-4.09 (m, 2H), 6.90-6.96 (m, 1H), 7.03 (s, 1H), 7.39 (d, J=8.40 Hz, 1H), 7.43 (br s, 1H), 7.70 (br s, 1H). Mass spec m/z 382.9 [M+H]⁺.

### Step 3

### Intermediate 833: 2-chloro-4-(2-(piperidin-4-yl)ethoxy)benzamide hydrochloride

To a solution of tert-butyl 4-(2-(4-carbamoyl-3-chlorophenoxy)ethyl)piperidine-1-carboxylate (Intermediate 832, 9.0 g, 23.5 mmol) in 1,4-dioxane (100 mL) was added 4M hydrochloric acid in 1,4-dioxane (100 mL). The reaction mixture was stirred at room temperature for 4 h. The reaction mixture was then concentrated in vacuo to afford 2-chloro-4-(2-(piperidin-4-yl)ethoxy)benzamide hydrochloride (Intermediate 833, 9.0 g) as an off-white solid, which was used for the next step without further purification. ¹H NMR (401 MHz, DMSO-d₆) δ ppm 1.30-1.42 (m, 2H), 1.62-1.84 (s, 5H), 2.75-2.89 (m, 2H), 3.17-3.24 (m, 2H), 3.90-4.07 (m, 4H), 6.91 (d, J=8.80, 2.40 Hz, 1H), 7.12 (d, J=2.4 Hz, 1H), 7.38-7.45 (m, 2H), 7.68 (br s, 1H). Mass spec m/z 283.0 [M+H]⁺.

### Step 4

### Intermediate 834: 4-(2-(1-(3-bromo-2-formylphenyl)piperidin-4-yl)ethoxy)-2-chlorobenzamide

To a solution of 2-chloro-4-(2-(piperidin-4-yl)ethoxy)benzamide hydrochloride (Intermediate 833, 2.0 g, 7.07 mmol) in dimethyl sulfoxide (20 mL) was added N,N-diisopropylethylamine (5.47 mL, 31.3 mmol) followed by 2-bromo-6-fluorobenzaldehyde (CAS No. 360575-28-6, 1.27 g, 7.07 mmol) and the reaction mixture was heated to 70° C. for 16 h. The reaction mixture was quenched with water (250 mL) and extracted with ethyl acetate (2×125 mL). The combined organic layers were dried over Na₂SO₄, filtered and concentrated in vacuo. The crude material was purified by combi-flash chromatography, by eluting with 30% ethyl acetate in heptane, to afford benzyl 4-(2-(1-(3-bromo-2-formylphenyl)piperidin-4-yl)ethoxy)-2-chlorobenzamide (Intermediate 834, 1.1 g, 33%) as a pale yellow semi-solid. ¹H NMR (401 MHz, DMSO-d₆) δ ppm 1.38-1.44 (m, 2H), 1.58-1.84 (s, 5H), 2.79-2.89 (m, 2H), 3.14-3.19 (m, 2H), 4.06-4.10 (m, 2H), 6.91-6.95 (m, 1H), 7.04 (d, J=2.00 Hz, 1H), 7.20 (d, J=2.00 Hz, 1H), 7.29 (d, J=7.60 Hz, 1H), 7.36-7.42 (m, 3H), 7.68 (s, 1H), 10.02 (s, 1H). Mass spec m/z 466.9 [M+H]⁺.

### Step 5

### Intermediate 835: 4-(2-(1-(3-bromo-2-(((2,6-dioxopiperidin-3-yl)amino)methyl)phenyl)piperidin-4-yl)ethoxy)-2-chlorobenzamide

To a solution of 4-(2-(1-(3-bromo-2-formylphenyl)piperidin-4-yl)ethoxy)-2-chlorobenzamide (Intermediate 834, 1.0 g, 2.14 mmol) in dimethylformamide (10 mL) was added 3-aminopiperidine-2,6-dione hydrochloride (CAS No. 24666-56-6, 0.42 g, 2.57 mmol) followed by triethylamine (0.9 mL, 6.44 mmol) and the reaction mixture was stirred at 70° C. for 2 h. Sodium cyanoborohydride (0.404 g, 6.44 mmol) was then added and reaction mixture was heated at 70° C. for 2 h. The reaction mixture was quenched with water (100 mL) and extracted with ethyl acetate (3×100 mL). The combined organic layers were dried over Na₂SO₄, filtered, and concentrated in vacuo. The crude material was purified by combi-flash chromatography, by eluting with 10% MeOH in DCM, to afford 4-(2-(1-(3-bromo-2-(((2,6-dioxopiperidin-3-yl)amino)methyl)phenyl)piperidin-4-yl)ethoxy)-2-chlorobenzamide (Intermediate 835, 1.0 g, 81%) as a green semi-solid. ¹H NMR (401 MHz, DMSO-d₆) δ ppm 1.30-1.42 (m, 2H), 1.55-1.85 (m, 8H), 2.26-2.34 (m, 1H), 2.52-2.57 (m, 2H), 2.72-2.79 (m, 1H), 2.87-2.96 (m, 1H), 3.04-3.12 (m, 1H), 3.20-3.25 (m, 1H), 3.86-3.95 (m, 2H), 4.07-4.11 (m, 2H), 6.95 (d, J=8.80, 2.4 Hz, 1H), 7.05 (d, J=2.00 Hz, 1H), 7.13-7.20 (m, 2H), 7.32 (d, J=7.20 Hz, 1H), 7.38-7.46 (m, 2H), 7.71 (s, 1H), 10.80 (s, 1H). Mass spec m/z 579.0 [M+H]⁺.

### Step 6

### Intermediate 836: 2-chloro-4-(2-(1-(2-(2,6-dioxopiperidin-3-yl)-1-oxoisoindolin-4-yl)piperidin-4-yl)ethoxy)benzamide

To a solution of 4-(2-(1-(3-bromo-2-(((2,6-dioxopiperidin-3-yl)amino)methyl)phenyl)piperidin-4-yl)ethoxy)-2-chlorobenzamide (Intermediate 835, 1.0 g, 1.73 mmol) in 1,4-dioxane (10 mL) was added triethylamine (0.72 mL, 5.19 mmol). The reaction mixture was purged with argon gas for 15 min then Xantphos (0.2 g, 0.34 mmol), PdCl₂(dppf) (0.25 g, 0.34 mmol) and tungsten hexacarbonyl (0.30 g, 0.86 mmol) were added. The reaction mixture was further purged with argon gas for 10 min and heated at 100° C. for 16 h. The reaction mixture was filtered through a celite bed and the filter pad was washed with ethyl acetate (200 mL). The filtrate was concentrated in vacuo and the crude material purified by combi-flash chromatography, by eluting with 100% ethyl acetate in heptane, to afford 2-chloro-4-(2-(1-(2-(2,6-dioxopiperidin-3-yl)-1-oxoisoindolin-4-yl)piperidin-4-yl)ethoxy)benzamide (Intermediate 836, 0.8 g, 89%) as a brown solid. ¹H NMR (401 MHz, DMSO-d₆) δ ppm 1.32-1.40 (m, 2H), 1.61-1.81 (m, 7H), 1.97-2.01 (m, 2H), 2.52-2.56 (m, 1H), 2.70-2.78 (m, 2H), 3.34-3.38 (m, 1H), 4.06-4.11 (m, 2H), 4.24-4.35 (m, 1H), 4.40-4.47 (m, 1H), 5.11-5.15 (m, 1H), 6.95 (dd, J=8.80, 2.4 Hz, 1H), 7.50 (d, J=2.00 Hz, 1H), 7.16 (d, J=8.00 Hz, 1H), 7.29 (d, J=7.20 Hz, 1H), 7.39-7.45 (m, 3H), 7.71 (br s, 1H), 10.98 (s, 1H). Mass spec m/z 525.2 [M+H]⁺.

### Step 7

### Intermediate 837: tert-butyl 6-(2-chloro-4-(2-(1-(2-(2,6-dioxopiperidin-3-yl)-1-oxoisoindolin-4-yl)piperidin-4-yl)ethoxy)benzamido)-2-((R)-1-methylpyrrolidin-2-yl)-1H-pyrrolo[3,2-c]pyridine-1-carboxylate

To a solution of 2-chloro-4-(2-(1-(2-(2,6-dioxopiperidin-3-yl)-1-oxoisoindolin-4-yl)piperidin-4-yl)ethoxy)benzamide (Intermediate 836, 0.5 g, 0.95 mmol) in 1,4-dioxane (20 mL) was added tert-butyl (R)-6-bromo-2-(1-methylpyrrolidin-2-yl)-1H-pyrrolo[3,2-c]pyridine-1-carboxylate (Intermediate 4, 0.066 g, 0.17 mmol) followed by cesium carbonate (0.93 g, 2.85 mmol). The reaction mixture was purged with argon for 15 min, then Pd₂(dba)₃ (0.13 g, 0.14 mmol) and Xantphos (0.16 g, 0.28 mmol) were added. The reaction mixture was purged with argon for another 10 min and stirred at 100° C. for 2 h. The reaction mixture was filtered through a celite bed, the filter pad was washed with ethyl acetate (100 mL) and the filtrate concentrated in vacuo. The crude material was purified by combi-flash chromatography, by eluting with 9% MeOH in DCM to afford tert-butyl 6-(2-chloro-4-(2-(1-(2-(2,6-dioxopiperidin-3-yl)-1-oxoisoindolin-4-yl)piperidin-4-yl)ethoxy)benzamido)-2-((R)-1-methylpyrrolidin-2-yl)-1H-pyrrolo[3,2-c]pyridine-1-carboxylate (Intermediate 837, 0.6 g, 76%) as an off-white solid.

Mass spec m/z 824.4 [M+H]⁺.

### Step 8

### Example 205: 2-chloro-4-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide

To a solution of tert-butyl 6-(2-chloro-4-(2-(1-(2-(2,6-dioxopiperidin-3-yl)-1-oxoisoindolin-4-yl)piperidin-4-yl)ethoxy)benzamido)-2-((R)-1-methylpyrrolidin-2-yl)-1H-pyrrolo[3,2-c]pyridine-1-carboxylate (Intermediate 837, 0.6 g, 0.72 mmol) in 1,4-dioxane (30 mL) was added 4M hydrochloric acid in 1,4-dioxane (30 mL). The reaction mixture was stirred at room temperature for 4 h. The reaction mixture was then concentrated in vacuo and the crude material was purified by preparative HPLC (Method A) to afford 2-chloro-4-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide (Example 205, 0.117 g, 32%) as an off-white solid. ¹H NMR (401 MHz, DMSO-d₆) δ ppm 1.33-1.44 (m, 2H), 1.61-2.02 (m, 9H), 2.12-2.18 (m, 4H), 2.21-2.28 (m, 1H), 2.52-2.61 (m, 2H), 2.71-2.89 (m, 2H), 2.90-2.97 (m, 1H), 3.11-3.18 (m, 1H), 3.27-3.29 (m, 1H), 3.32-3.42 (m, 2H), 4.11-4.16 (m, 2H), 4.27-4.32 (m, 1H), 4.42-4.46 (m, 1H), 5.09-5.14 (m, 1H), 6.38 (s, 1H), 6.99 (dd, J=8.80, 2.4 Hz, 1H), 7.11 (d, J=2.00 Hz, 1H), 7.17 (d, J=7.60 Hz, 1H), 7.30 (d, J=7.20 Hz, 1H), 7.43 (t, J=8.00 Hz, 1H), 7.51 (d, J=8.800 Hz, 1H), 8.17 (s, 1H), 8.45 (s, 1H), 10.48 (s, 1H), 10.98 (s, 1H), 11.35 (s, 1H). Mass spec m/z 724.4 [M+H]⁺.

### Example 206 was Prepared Following Scheme 199

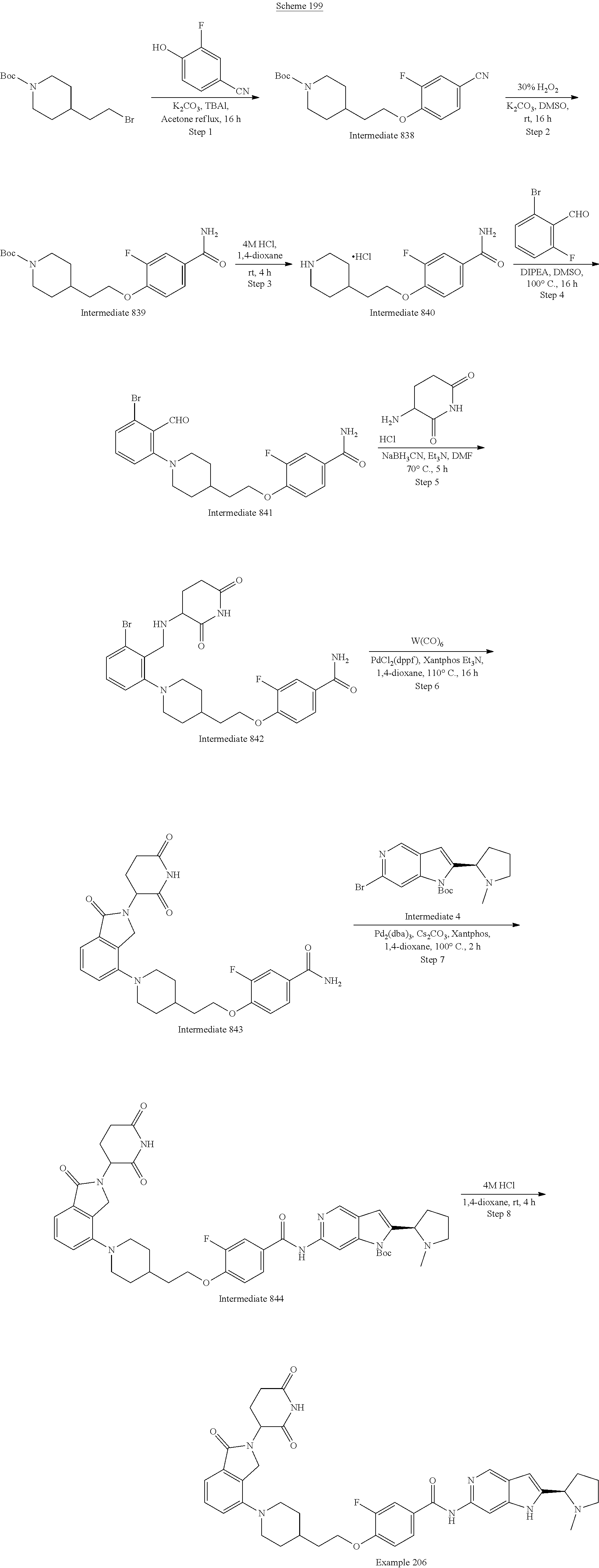

### Step 1

### Intermediate 838: tert-butyl 4-(2-(4-cyano-2-fluorophenoxy)ethyl)piperidine-1-carboxylate

To a solution of 3-fluoro-4-hydroxybenzonitrile (CAS No. 405-04-9, 4.69 g, 34.2 mmol) in acetone (100 mL) was added tert-butyl 4-(2-bromoethyl)piperidine-1-carboxylate (CAS No. 169457-73-2, 10.0 g, 34.2 mmol) followed by potassium carbonate (18.9 g, 137 mmol) and tetrabutylammonium iodide (1.28 g 3.42 mmol). The reaction mixture was heated at 50° C. for 16 h then concentrated in vacuo. The crude material was purified by combi-flash chromatography, by eluting with 30% ethyl acetate in heptane, to afford tert-butyl 4-(2-(4-cyano-2-fluorophenoxy)ethyl)piperidine-1-carboxylate (Intermediate 838, 8.0 g, 67%) as an off-white solid. ¹H NMR (400 MHz, DMSO-d₆) δ ppm 0.98-1.10 (m, 2H), 1.36 (s, 9H), 1.57-1.71 (m, 5H), 2.60-2.78 (m, 2H), 3.86-3.92 (m, 2H), 4.13-4.20 (m, 2H), 7.35 (t, J=8.40 Hz, 1H), 7.65 (d, J=8.40 Hz, 1H), 7.81 (d, J=11.6 Hz, 1H).

### Step 2

### Intermediate 839: tert-butyl 4-(2-(4-carbamoyl-2-fluorophenoxy)ethyl)piperidine-1-carboxylate

To a cooled (0° C.) solution of tert-butyl 4-(2-(4-cyano-2-fluorophenoxy)ethyl)piperidine-1-carboxylate (Intermediate 838, 8.0 g, 23.0 mmol) in dimethyl sulfoxide (80 mL) was added potassium carbonate (9.52 g, 68.9 mmol) followed by 30% hydrogen peroxide (2.81 mL, 91.8 mmol) and the reaction mixture was stirred at room temperature for 16 h. The reaction mixture was then quenched with ice cold water (50 mL), the resultant precipitate was collected by filtration and dried in vacuo to afford tert-butyl 4-(2-(4-carbamoyl-2-fluorophenoxy)ethyl)piperidine-1-carboxylate (Intermediate 839, 8.50 g) as an off-white solid, which was used for the next step without further purification. ¹H NMR (400 MHz, DMSO-d₆) δ ppm 0.91-0.98 (m, 2H), 1.42 (s, 9H), 2.52-2.57 (m, 2H), 2.93-3.03 (m, 2H), 3.12-3.21 (m, 1H), 3.42-3.51 (m, 1H), 3.74-3.83 (m, 1H), 3.90-3.41 (m, 1H), 6.88 (d, J=8.22 Hz, 2H), 7.01 (br s, 1H), 7.69 (br s, 1H), 7.75 (d, J=7.83 Hz, 2H). Mass spec m/z 365.0 [M−H]⁻.

### Step 3

### Intermediate 840: 3-fluoro-4-(2-(piperidin-4-yl)ethoxy)benzamide hydrochloride

To a cooled (0° C.) solution of tert-butyl 4-(2-(4-carbamoyl-2-fluorophenoxy)ethyl)piperidine-1-carboxylate (Intermediate 839, 8.0 g, 21.8 mmol) in 1,4-dioxane (80 mL) was added 4M hydrochloric acid in 1,4-dioxane (80 mL) and the reaction mixture was stirred at room temperature for 4 h. The reaction mixture was then concentrated in vacuo to afford 3-fluoro-4-(2-(piperidin-4-yl)ethoxy)benzamide hydrochloride (Intermediate 840, 5.5 g, 95%) as an off-white solid, which was used for the next step without further purification. ¹H NMR (400 MHz, DMSO-d₆) δ ppm 1.35-1.39 (m, 2H), 1.70-1.78 (m, 3H), 1.83-1.86 (m, 2H), 2.75-2.87 (m, 2H), 3.17-3.24 (m, 2H), 4.13-4.16 (m, 2H), 7.23 (t, J=8.40 Hz, 1H), 7.32 (br s, 1H), 7.70-7.54 (m, 2H), 7.94 (br s, 1H). Mass spec m/z 267.1 [M+H]⁺.

### Step 4

### Intermediate 841: 4-(2-(1-(3-bromo-2-formylphenyl)piperidin-4-yl)ethoxy)-3-fluorobenzamide

To a solution of 3-fluoro-4-(2-(piperidin-4-yl)ethoxy)benzamide hydrochloride (Intermediate 840, 2.0 g, 7.51 mmol) in dimethyl sulfoxide (20 mL) was added N,N-diisopropylethylamine (5.25 mL, 30.0 mmol) followed by 2-bromo-6-fluorobenzaldehyde (CAS No. 360575-28-6, 1.25 g, 7.51 mmol) and the reaction mixture was heated at 100° C. for 16 h. The reaction mixture was then diluted with water (100 mL) and extracted with ethyl acetate (2×125 mL). The combined organic layers were dried over anhydrous Na₂SO₄, filtered and concentrated in vacuo. The crude material was purified by combi-flash chromatography, by eluting with 90% ethyl acetate in heptane, to afford 4-(2-(1-(3-bromo-2-formylphenyl)piperidin-4-yl)ethoxy)-3-fluorobenzamide (Intermediate 841, 1.0 g, 30%) as an off-white solid. ¹H NMR (400 MHz, DMSO-d6) δ ppm 1.38-1.47 (m, 2H), 1.59-1.60 (m, 1H), 1.74-1.84 (m, 4H), 2.82-2.89 (m, 2H), 3.15-3.20 (m, 2H), 4.15-4.21 (m, 2H), 7.21-7.35 (m, 4H), 7.38-7.44 (m, 1H), 7.66-7.72 (m, 2H), 7.91 (br s, 1H), 10.38 (s, 1H). Mass spec m/z 451.1 [M+H+2]⁺.

### Step 5

### Intermediate 842: 4-(2-(1-(3-bromo-2-(((2,6-dioxopiperidin-3-yl)amino)methyl)phenyl)piperidin-4-yl)ethoxy)-3-fluorobenzamide

To a solution of 4-(2-(1-(3-bromo-2-formylphenyl)piperidin-4-yl)ethoxy)-3-fluorobenzamide (Intermediate 841, 1.0 g, 2.22 mmol) in dimethylformamide (10 mL) were added 3-aminopiperidine-2,6-dione hydrochloride (CAS No. CAS No. 24666-56-6, 2.22 g, 2.22 mmol) and triethylamine (0.93 mL, 6.67 mmol) and the reaction mixture was heated at 70° C. for 2 h. Sodium cyanoborohydride (0.44 g, 6.67 mmol) was then added and the reaction mixture was heated at 70° C. for 5 h. The reaction mixture was then diluted with water (50 mL) and extracted with ethyl acetate (3×50 mL). The combined organic layers were dried over anhydrous Na₂SO₄ filtered, and concentrated in vacuo. The crude material was purified by combi-flash chromatography, by eluting with 100% ethyl acetate, to afford 4-(2-(1-(3-bromo-2-(((2,6-dioxopiperidin-3-yl)amino)methyl)phenyl)piperidin-4-yl)ethoxy)-3-fluorobenzamide (Intermediate 842, 1.0 g, 80%) as a white solid. ¹H NMR (400 MHz, DMSO-d6) δ ppm 1.07-1.14 (m, 1H), 1.31-1.45 (m, 2H), 1.51-1.89 (m, 6H), 2.27-2.35 (m, 1H), 2.53-2.60 (m, 2H), 2.73-2.95 (m, 4H), 3.06-3.11 (m, 1H), 3.20-3.32 (m, 1H), 3.85-3.96 (m, 1H), 4.16-4.20 (m, 2H), 7.13-7.34 (m, 5H), 7.68-7.72 (m, 2H), 7.89-7.96 (m, 1H), 10.79 (s, 1H). Mass spec m/z 563.2 [M+H+2]⁺.

### Step 6

### Intermediate 843: 4-(2-(1-(2-(2,6-dioxopiperidin-3-yl)-1-oxoisoindolin-4-yl)piperidin-4-yl)ethoxy)-3-fluorobenzamide

To a solution of 4-(2-(1-(3-bromo-2-(((2,6-dioxopiperidin-3-yl)amino)methyl)phenyl)piperidin-4-yl)ethoxy)-3-fluorobenzamide (Intermediate 842, 1.0 g, 1.78 mmol) in 1,4-dioxane (10 mL) was added triethylamine (0.74 mL, 5.34 mmol). The reaction mixture was purged with argon for 15 min, then Xantphos (0.31 g, 0.53 mmol) followed by PdCl₂(dppf) (0.27 g, 0.35 mmol) and W(CO)₆ (0.32 g, 0.89 mmol) were added. The reaction mixture was further purged with argon for 10 min and heated at 110° C. for 16 h. The reaction mixture was then filtered through a celite bed and the filter pad was washed with ethyl acetate (200 mL). The filtrate was concentrated in vacuo and the crude material was purified by combi-flash chromatography, by eluting with 100% ethyl acetate, to afford 4-(2-(1-(2-(2,6-dioxopiperidin-3-yl)-1-oxoisoindolin-4-yl)piperidin-4-yl)ethoxy)-3-fluorobenzamide (Intermediate 843, 0.6 g, 66%) as a brown solid. ¹H NMR (400 MHz, DMSO-d₆) δ ppm 1.13-1.18 (m, 2H), 1.32-1.40 (m, 2H), 1.55-1.89 (m, 4H), 1.91-2.00 (m, 2H), 2.61-2.74 (m, 2H), 2.81-2.91 (m, 1H), 3.33-3.41 (m, 1H), 3.96-4.05 (m, 1H), 4.12-4.20 (m, 2H), 4.25-4.29 (m, 1H), 4.39-4.44 (m, 1H), 5.07-5.12 (m, 1H), 7.13-7.17 (m, 1H), 7.22-7.34 (m, 3H), 7.35-7.42 (m, 1H), 7.64-7.71 (m, 2H), 7.88 (bs, 1H), 10.96 (s, 1H). Mass spec m/z 509.2 [M+H]⁺.

### Step 7

### Intermediate 844: tert-butyl 6-(4-(2-(1-(2-(2,6-dioxopiperidin-3-yl)-1-oxoisoindolin-4-yl)piperidin-4-yl)ethoxy)-3-fluorobenzamido)-2-((R)-1-methylpyrrolidin-2-yl)-1H-pyrrolo[3,2-c]pyridine-1-carboxylate

To a solution of 4-(2-(1-(2-(2,6-dioxopiperidin-3-yl)-1-oxoisoindolin-4-yl)piperidin-4-yl)ethoxy)-3-fluorobenzamide (Intermediate 843, 0.53 g, 1.02 mmol) in 1,4-dioxane (20 mL) was added tert-butyl (R)-6-bromo-2-(1-methylpyrrolidin-2-yl)-1H-pyrrolo[3,2-c]pyridine-1-carboxylate (Intermediate 4, 0.50 g, 1.31 mmol) followed by cesium carbonate (1.29 g, 3.94 mmol). The reaction mixture was purged with argon for 15 min, then Pd₂(dba)₃ (0.18 g, 0.19 mmol) followed by Xantphos (0.23 g, 0.39 mmol) were added. The reaction mixture was further purged with argon for 10 min and heated at 100° C. for 2 h. The reaction mixture was then filtered through a celite bed, the filter pad was washed with ethyl acetate (100 mL) and the filtrate concentrated in vacuo. The crude material was purified by combi-flash chromatography, by eluting with 90% ethyl acetate in heptane, to afford tert-butyl 6-(4-(2-(1-(2-(2,6-dioxopiperidin-3-yl)-1-oxoisoindolin-4-yl)piperidin-4-yl)ethoxy)-3-fluorobenzamido)-2-((R)-1-methylpyrrolidin-2-yl)-1H-pyrrolo[3,2-c]pyridine-1-carboxylate (Intermediate 844, 0.50 g, 47%) as an off-white solid. ¹H NMR (400 MHz, DMSO-d₆) δ ppm 1.43-1.52 (m, 3H), 1.56-1.89 (m, 18H), 1.95-2.01 (m, 1H), 2.29-2.39 (m, 4H), 2.53-2.62 (m, 1H), 2.71-2.78 (m, 2H), 2.81-2.91 (m, 1H), 3.10-3.15 (m, 1H), 3.38-3.50 (m, 2H), 3.87-3.94 (m, 1H), 4.18-4.32 (m, 3H), 4.42-4.46 (m, 1H), 5.08-5.14 (m, 1H), 6.75 (s, 1H), 7.11-7.45 (m, 4H), 7.92-7.95 (m, 2H), 8.59 (s, 1H), 8.90 (m, 1H), 10.66 (s, 1H), 10.97 (s, 1H). Mass spec m/z 808.4 [M+H]⁺.

### Step 8

### Example 206: 4-(2-(1-(2-(2,6-dioxopiperidin-3-yl)-1-oxoisoindolin-4-yl)piperidin-4-yl)ethoxy)-3-fluoro-N-(2-((R)-1-methylpyrrolidin-2-yl)-1H-pyrrolo[3,2-c]pyridin-6-yl)benzamide

To a cooled (0° C.) solution of tert-butyl 6-(4-(2-(1-(2-(2,6-dioxopiperidin-3-yl)-1-oxoisoindolin-4-yl)piperidin-4-yl)ethoxy)-3-fluorobenzamido)-2-((R)-1-methylpyrrolidin-2-yl)-1H-pyrrolo[3,2-c]pyridine-1-carboxylate (Intermediate 844, 0.50 g, 0.61 mmol) in 1,4-dioxane (5 mL) was added 4M hydrochloric acid in 1,4-dioxane (5 mL) and the reaction mixture was stirred at room temperature for 4 h. The reaction mixture was then concentrated in vacuo and the crude material purified by preparative HPLC (Method A) to afford 4-(2-(1-(2-(2,6-dioxopiperidin-3-yl)-1-oxoisoindolin-4-yl)piperidin-4-yl)ethoxy)-3-fluoro-N-(2-((R)-1-methylpyrrolidin-2-yl)-1H-pyrrolo[3,2-c]pyridin-6-yl)benzamide (Example 206, 0.10 g, 24%) as pale yellow solid. ¹H NMR (400 MHz, DMSO-d₆) δ ppm 1.32-1.41 (m, 2H), 1.61-1.70 (m, 1H), 1.74-1.89 (m, 7H), 1.96-2.01 (m, 1H), 2.13-2.18 (m, 4H), 2.21-2.28 (m, 1H), 2.41-2.48 (m, 2H), 2.56-2.61 (m, 1H), 2.71-2.81 (m, 2H), 2.89-2.91 (m, 1H), 3.12-3.16 (m, 1H), 3.32-3.41 (m, 2H), 4.21-4.25 (m, 2H), 4.28-4.32 (m, 1H), 4.42-4.46 (m, 1H), 5.09-5.14 (m, 1H), 6.39 (s, 1H), 7.17 (d, J=7.6 Hz, 1H), 7.29-7.31 (m, 2H), 7.41-7.45 (m, 1H), 7.92-7.96 (m, 2H), 8.16 (s, 1H), 8.50 (m, 1H), 10.43 (s, 1H), 10.97 (s, 1H), 11.36 (s, 1H). Mass spec m/z 706.2 [M−H]⁻.

### Example 207 was Prepared Following Scheme 200

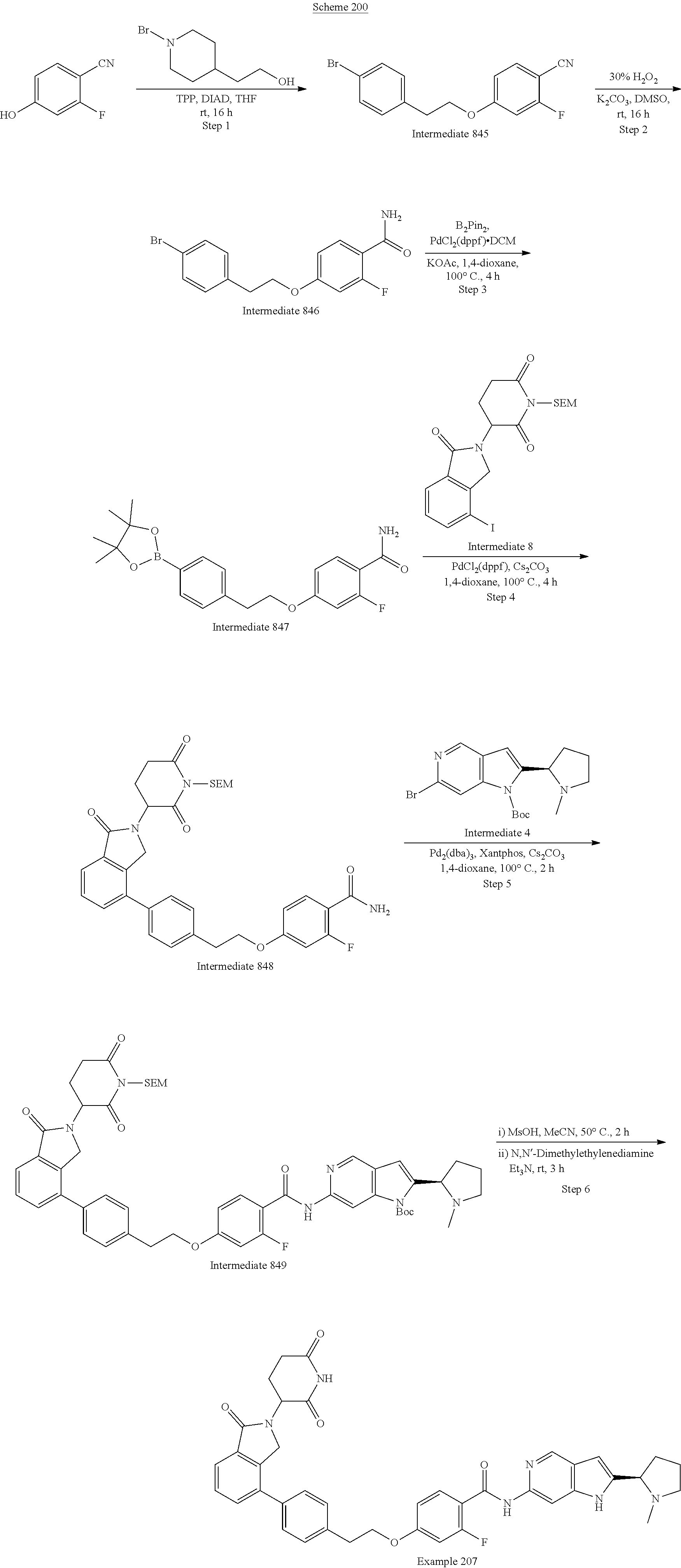

### Step 1

### Intermediate 845: 4-(4-bromophenethoxy)-2-fluorobenzonitrile

To a solution of 2-fluoro-4-hydroxybenzonitrile (CAS No. 82380-18-5, 10.9 g, 79.6 mmol) in tetrahydrofuran (120 mL) was added 2-(4-bromophenyl)ethan-1-ol (CAS No 4654-39-1, 16.0 g, 79.6 mmol), triphenylphosphine (27.6 g, 103 mmol) and diisopropyl azodicarboxylate (20.8 mL 103 mmol) and the reaction mixture was stirred at room temperature for 16 h. The reaction mixture was then concentrated in vacuo and the crude material was purified by combi-flash chromatography, by eluting with 15% ethyl acetate in heptane, to afford 4-(4-bromophenethoxy)-2-fluorobenzonitrile (Intermediate 845, 10.0 g, 79%) as an off-white solid. ¹H NMR (400 MHz, DMSO-d₆) δ ppm 3.04-3.08 (m, 2H), 4.16-4.19 (m, 2H), 6.66-6.74 (m, 2H), 7.13 (d, J=8.40 Hz, 2H), 7.43-7.51 (m, 3H).

### Step 2

### Intermediate 846: 4-(4-bromophenethoxy)-2-fluorobenzamide

To a cooled (0° C.) solution of 4-(4-bromophenethoxy)-2-fluorobenzonitrile (Intermediate 845, 20.0 g, 59.5 mmol) in dimethyl sulfoxide (70 mL) was added potassium carbonate (18.9 g, 137 mmol) followed by 30% hydrogen peroxide (14.1 mL, 595 mmol) and the reaction mixture was stirred at room temperature for 16 h. The reaction mixture was then quenched with ice cold water (500 mL), the resultant precipitate was collected by filtration and dried in vacuo to afford 4-(4-bromophenethoxy)-2-fluorobenzamide (Intermediate 846, 15.0 g, 74%) as an off-white solid, which was used for the next step without further purification. ¹H NMR (400 MHz, DMSO-d₆) δ ppm 3.00-3.04 (m, 2H), 4.23-4.27 (m, 2H), 6.81-6.90 (m, 2H), 7.28 (d, J=8.40 Hz, 2H), 7.39-7.51 (m, 4H), 7.65 (t, J=9.2 Hz, 1H). Mass spec m/z 339.9 [M+H+2]⁺.

### Step 3

### Intermediate 847: 2-fluoro-4-(4-(4,4,5,5-tetramethyl-1,3,2-dioxaborolan-2-yl)phenethoxy)benzamide

To a solution of 4-(4-bromophenethoxy)-2-fluorobenzamide (Intermediate 846, 5.0 g, 14.8 mmol) in 1,4-dioxane (50 mL) was added 4,4,4′,4′,5,5,5′,5′-octamethyl-2,2′-bi(1,3,2-dioxaborolane) (4.64 g, 17.7 mmol) followed by potassium acetate (4.40 g, 44.4 mmol). The reaction mixture was purged with argon for 15 min then PdCl₂(dppf). CH₂Cl₂ (1.23 g, 1.47 mmol) was added. The reaction mixture was further purged with argon for 10 min and heated at 100° C. for 4 h. The reaction mixture was then diluted with ice cold water (100 mL). The resultant precipitate was collected by filtration and dried in vacuo to afford 2-fluoro-4-(4-(4,4,5,5-tetramethyl-1,3,2-dioxaborolan-2-yl)phenethoxy)benzamide (Intermediate 847, 4.72 g, 83%) as a white solid, which was used for the next step without further purification. ¹H NMR (400 MHz, DMSO-d₆) δ ppm 1.26 (s, 12H), 3.01-3.05 (m, 2H), 4.21-4.26 (m, 2H), 6.68-6.86 (m, 2H), 7.29-7.33 (m, 2H), 7.37-7.46 (m, 2H), 7.58-7.64 (m, 3H). Mass spec m/z 386.1 [M+H]⁺.

### Step 4

### Intermediate 848: 4-(4-(2-(2,6-dioxo-1-((2-(trimethylsilyl)ethoxy)methyl)piperidin-3-yl)-1-oxoisoindolin-4-yl)phenethoxy)-2-fluorobenzamide

To a solution of 2-fluoro-4-(4-(4,4,5,5-tetramethyl-1,3,2-dioxaborolan-2-yl)phenethoxy)benzamide (Intermediate 847, 2.0 g, 5.19 mmol) in 1,4-dioxane (25 mL) was added 3-(4-iodo-1-oxoisoindolin-2-yl)-1-((2-(trimethylsilyl)ethoxy)methyl)piperidine-2,6-dione (Intermediate 8, 2.86 g, 5.19 mmol) followed by cesium carbonate (5.08 g, 15.6 mmol). The reaction mixture was purged with argon for 15 min then PdCl₂(dppf) (0.4 g, 0.51 mmol) was added. The reaction mixture was further purged with argon for 10 min and heated at 100° C. for 4 h. The reaction mixture was then concentrated in vacuo. The crude material was purified by combi-flash chromatography, by eluting with 80% ethyl acetate in heptane, to afford 4-(4-(2-(2,6-dioxo-1-((2-(trimethylsilyl)ethoxy)methyl)piperidin-3-yl)-1-oxoisoindolin-4-yl)phenethoxy)-2-fluorobenzamide (Intermediate 848, 2.0 g, 61%) as an off-white solid. ¹H NMR (400 MHz, DMSO-d₆) δ ppm −0.07 (s, 9H), 0.78-0.83 (m, 2H), 2.06-2.09 (m, 2H), 2.42-2.49 (m, 1H), 2.78-3.01 (m, 1H), 3.05-3.13 (m, 2H), 3.47-3.51 (m, 2H), 4.28-4.36 (m, 3H), 4.64-4.68 (m, 1H), 4.99-5.06 (m, 2H), 5.25-5.30 (m, 1H), 6.84-6.92 (m, 2H), 7.38-7.47 (m, 4H), 7.54 (d, J=8.80 Hz, 2H), 7.62-7.71 (m, 3H), 7.76 (m, 1H).

### Step 5

### Intermediate 849: tert-butyl 6-(4-(4-(2-(2,6-dioxo-1-((2-(trimethylsilyl)ethoxy)methyl)piperidin-3-yl)-1-oxoisoindolin-4-yl)phenethoxy)-2-fluorobenzamido)-2-((R)-1-methylpyrrolidin-2-yl)-1H-pyrrolo[3,2-c]pyridine-1-carboxylate

To a solution of 4-(4-(2-(2,6-dioxo-1-((2-(trimethylsilyl)ethoxy)methyl)piperidin-3-yl)-1-oxoisoindolin-4-yl)phenethoxy)-2-fluorobenzamide (Intermediate 848, 0.80 g, 1.20 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 0.48 g, 1.36 mmol) followed by cesium carbonate (1.20 g, 3.76 mmol). The reaction mixture was purged with argon for 15 min, then Pd₂(dba)₃ (0.16 g, 0.16 mmol) followed by Xantphos (0.16 g, 0.32 mmol) were added. The reaction mixture was further purged with argon for 10 min and heated at 100° C. for 2 h. The reaction mixture was filtered through a celite bed, the filter pad was washed with ethyl acetate (100 mL) and the filtrate concentrated in vacuo. The crude material was purified by combi-flash chromatography, by eluting with 5% MeOH in DCM, to afford tert-butyl 6-(4-(4-(2-(2,6-dioxo-1-((2-(trimethylsilyl)ethoxy)methyl)piperidin-3-yl)-1-oxoisoindolin-4-yl)phenethoxy)-2-fluorobenzamido)-2-((R)-1-methylpyrrolidin-2-yl)-1H-pyrrolo[3,2-c]pyridine-1-carboxylate (Intermediate 849, 0.4 g, 34%) as an off-white solid. ¹H NMR (400 MHz, DMSO-d₆) δ ppm −0.07 (s, 9H), 0.81-1.15 (m, 2H), 1.61-1.99 (m, 15H), 1.98-2.04 (m, 1H), 2.30-2.44 (m, 6H), 3.03-3.17 (m, 3H), 3.45-3.55 (m, 2H), 3.89-3.94 (m, 1H), 4.33-4.37 (m, 2H), 4.64-4.68 (m, 1H), 4.98-5.06 (m, 2H), 5.24-5.26 (m, 1H), 6.74 (s, 1H), 6.89-6.99 (m, 2H), 7.45 (d, J=8.00 Hz, 2H), 7.55 (d, J=8.80 Hz, 2H), 7.62-7.78 (m, 4H), 8.55 (s, 1H), 8.91 (s, 1H), 10.25 (d, J=4.40 Hz, 1H). Mass spec m/z 931.5 [M+H]⁺.

### Step 6

### Example 207: 4-(4-(2-(2,6-dioxopiperidin-3-yl)-1-oxoisoindolin-4-yl)phenethoxy)-2-fluoro-N-(2-((R)-1-methylpyrrolidin-2-yl)-1H-pyrrolo[3,2-c]pyridin-6-yl)benzamide

To a solution of tert-butyl 6-(4-(4-(2-(2,6-dioxo-1-((2-(trimethylsilyl)ethoxy)methyl)piperidin-3-yl)-1-oxoisoindolin-4-yl)phenethoxy)-2-fluorobenzamido)-2-((R)-1-methylpyrrolidin-2-yl)-1H-pyrrolo[3,2-c]pyridine-1-carboxylate (Intermediate 849, 0.35 g, 0.37 mmol) in acetonitrile (4 mL) was added methanesulfonic acid (0.24 mL, 3.75 mmol) and the reaction mixture was heated at 60° C. for 2 h. The reaction mixture was cooled to room temperature, then N,N′-dimethylethylenediamine (0.22 mL, 1.87 mmol) followed by triethylamine (1.05 mL, 7.51 mmol) were added and the mixture was stirred at room temperature for 3 h. The reaction mixture was concentrated in vacuo and the resultant residue was diluted with water (50 mL). The resultant precipitate was collected by filtration and dried in vacuo. The crude solid was purified by preparative HPLC (Method A) to afford 4-(4-(2-(2,6-dioxopiperidin-3-yl)-1-oxoisoindolin-4-yl)phenethoxy)-2-fluoro-N-(2-((R)-1-methylpyrrolidin-2-yl)-1H-pyrrolo[3,2-c]pyridin-6-yl)benzamide (Example 207, 0.06 g, 23%) as an off-white solid. ¹H NMR (400 MHz, DMSO-d₆) δ ppm 1.73-2.02 (m, 4H), 2.11-2.19 (m, 4H), 2.22-2.29 (m, 1H), 2.39-2.47 (m, 1H), 2.52-2.60 (m, 2H), 2.84-2.95 (m, 1H), 3.10-3.16 (m, 3H), 4.33-4.44 (m, 3H), 4.60-4.65 (m, 1H), 5.11-5.16 (m, 1H), 6.38 (s, 1H), 6.91-7.01 (m, 2H), 7.48 (d, J=8.40 Hz, 2H), 7.56 (d, J=8.00 Hz, 2H), 7.64 (t, J=7.20 Hz, 1H), 7.70-7.77 (m, 3H), 8.18 (s, 1H), 8.46 (s, 1H), 9.97 (d, J=5.60 Hz, 1H), 10.96 (br s, 1H), 11.37 (s, 1H). Mass spec m/z 701.3 [M+H]⁺.

### Example 208 was Prepared Following Scheme 201

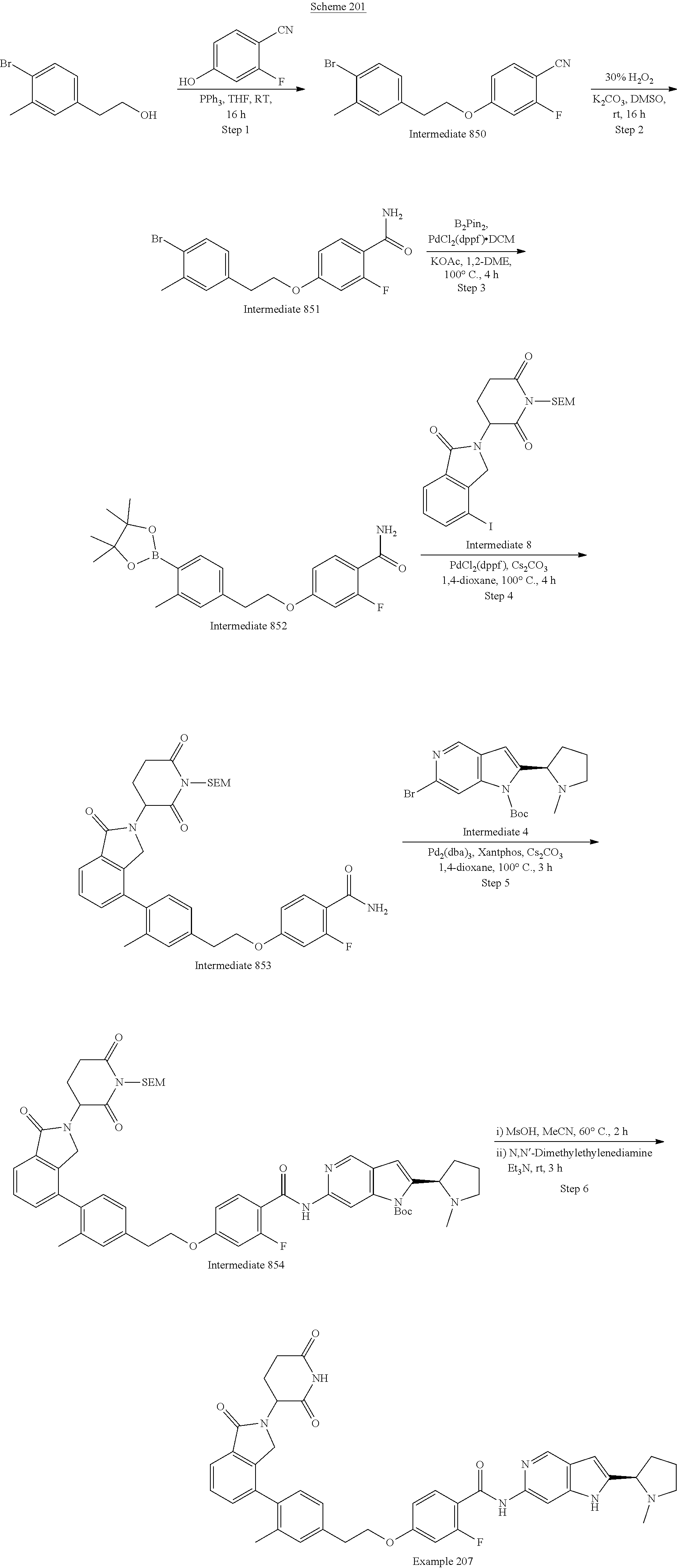

### Step 1

### Intermediate 850: 4-(4-bromo-3-methylphenethoxy)-2-fluorobenzonitrile

To a cooled (0° C.) solution of 2-fluoro-4-hydroxybenzonitrile (CAS No. 82380-18-5, 9.56 g, 69.7 mmol) in tetrahydrofuran (150 mL) were added 2-(4-bromo-3-methylphenyl)ethan-1-ol (CAS No. 1195716-51-8, 15.0 g, 69.7 mmol), triphenylphosphine (24.3 g, 90.7 mmol) and diisopropyl azodicarboxylate (18.70 g, 90.66 mmol). The reaction mixture was stirred at room temperature for 16 h. The reaction mixture was concentrated in vacuo and the crude material purified by combi-flash chromatography, by eluting with 50% ethyl acetate in heptane, to afford 4-(4-bromo-3-methylphenethoxy)-2-fluorobenzonitrile (Intermediate 850, 10.0 g, 43%) as an off-white semi-solid. ¹H NMR (400 MHz, DMSO-d₆) δ ppm 2.32 (s, 3H), 2.97-3.01 (m, 2H), 4.27-4.33 (m, 2H), 6.96 (dd, J=8.80, 2.00 Hz, 1H), 7.08 (d, J=6.80 Hz, 1H), 7.16 (dd, J=12.00, 2.00 Hz, 1H), 7.31 (br s, 1H), 7.49 (d, J=8.0 Hz, 1H), 7.79 (t, J=8.40 Hz, 1H).

### Step 2

### Intermediate 851: 4-(4-bromo-3-methylphenethoxy)-2-fluorobenzamide

To a solution of 4-(4-bromo-3-methylphenethoxy)-2-fluorobenzonitrile (Intermediate 850, 8.0 g, 23.9 mmol) in dimethyl sulfoxide (80 mL) was added potassium carbonate (8.25 g, 59.8 mmol) followed by 30% hydrogen peroxide (18.7 mL, 240 mmol) and the reaction mixture was stirred at room temperature for 16 h. The reaction mixture was quenched with ice cold water (500 mL), the resultant precipitate was collected by filtration and dried in vacuo to afford 4-(4-bromo-3-methylphenethoxy)-2-fluorobenzamide (Intermediate 851, 6.0 g, 71%) as an off-white solid, which was used for the next step without further purification. ¹H NMR (400 MHz, DMSO-d₆) δ ppm 2.32 (s, 3H), 2.95-3.01 (m, 2H), 4.21-4.26 (m, 2H), 6.81-6.90 (m, 2H), 7.09 (d, J=6.80 Hz, 1H), 7.32 (br s, 1H), 7.41-7.51 (m, 3H), 7.65 (m, 1H).

### Step 3

### Intermediate 852: 2-fluoro-4-(3-methyl-4-(4,4,5,5-tetramethyl-1,3,2-dioxaborolan-2-yl)phenethoxy)benzamide

To a solution of 4-(4-bromo-3-methylphenethoxy)-2-fluorobenzamide (Intermediate 851, 4.0 g, 11.4 mmol) 1,4-dioxane (40 mL) was added 4,4,4′,4′,5,5,5′,5′-octamethyl-2,2′-bi-1,3,2-dioxaborolane (3.56 g, 13.4 mmol) followed by potassium acetate (3.38 g, 34.1 mmol). The reaction mixture was purged with argon for 15 min, then PdCl₂(dppf) CH₂Cl₂ (0.94 g, 1.13 mmol) was added. The reaction mixture was further purged with argon for 10 min and heated at 100° C. for 4 h. The reaction mixture was quenched with ice cold water (150 mL), the resultant precipitate was collected by filtration and dried in vacuo to afford 2-fluoro-4-(3-methyl-4-(4,4,5,5-tetramethyl-1,3,2-dioxaborolan-2-yl)phenethoxy)benzamide (Intermediate 852, 4.2 g, 93%) as a white solid, which was used for the next step without further purification. ¹H NMR (400 MHz, DMSO-d₆) δ ppm 1.29 (s, 12H), 2.42 (s, 3H), 2.98-3.02 (m, 2H), 4.22-4.26 (m, 2H), 6.80-6.88 (m, 2H), 7.09-7.13 (m, 2H), 7.39-7.48 (m, 2H), 7.56 (d, J=7.20 Hz, 1H), 7.65 (t, J=8.80 Hz, 1H).

### Step 4

### Intermediate 853: 4-(4-(2-(2,6-dioxo-1-((2-(trimethylsilyl)ethoxy)methyl)piperidin-3-yl)-1-oxoisoindolin-4-yl)-3-methylphenethoxy)-2-fluorobenzamide

To a solution of 2-fluoro-4-(3-methyl-4-(4,4,5,5-tetramethyl-1,3,2-dioxaborolan-2-yl)phenethoxy)benzamide (Intermediate 852, 1.0 g, 2.50 mmol) in 1,4-dioxane (10 mL) were added 3-(4-iodo-1-oxoisoindolin-2-yl)-1-((2-(trimethylsilyl)ethoxy)methyl)piperidine-2,6-dione (Intermediate 8, 1.50 g, 3.0 mmol) followed by cesium carbonate (2.45 g, 7.51 mmol). The reaction mixture was purged with argon for 15 min then PdCl₂(dppf) (0.19 g, 0.25 mmol) was added. The reaction mixture was further purged with argon for 10 min and heated at 100° C. for 4 h. The reaction mixture was concentrated in vacuo. The crude material was purified by combi-flash chromatography, by eluting with 10% MeOH in DCM, to afford 4-(4-(2-(2,6-dioxo-1-((2-(trimethylsilyl)ethoxy)methyl)piperidin-3-yl)-1-oxoisoindolin-4-yl)-3-methylphenethoxy)-2-fluorobenzamide (Intermediate 853, 1.0 g, 62%) as an off-white solid. ¹H NMR (400 MHz, DMSO-d₆) δ ppm −0.79 (s, 9H), 0.75-0.82 (m, 2H), 1.97-2.02 (m, 1H), 2.09 (s, 3H), 2.26-2.39 (m, 1H), 2.72-2.75 (m, 1H), 2.96-3.07 (m, 3H), 3.42-3.52 (m, 2H), 4.00-4.05 (m, 1H), 4.19-4.32 (m, 3H), 4.95-5.05 (m, 2H), 5.23-5.28 (m, 1H), 6.84-6.93 (m, 2H), 7.17-7.25 (m, 2H), 7.32 (br s, 1H), 7.40-7.50 (m, 3H), 7.58-7.70 (m, 2H), 7.76-7.78 (m, 1H). Mass spec m/z 644.2 [M−H]⁻.

### Step 5

### Intermediate 854: tert-butyl 6-(4-(4-(2-(2,6-dioxo-1-((2-(trimethylsilyl)ethoxy)methyl)piperidin-3-yl)-1-oxoisoindolin-4-yl)-3-methylphenethoxy)-2-fluorobenzamido)-2-((R)-1-methylpyrrolidin-2-yl)-1H-pyrrolo[3,2-c]pyridine-1-carboxylate

To a solution of 4-(4-(2-(2,6-dioxo-1-((2-(trimethylsilyl)ethoxy)methyl)piperidin-3-yl)-1-oxoisoindolin-4-yl)-3-methylphenethoxy)-2-fluorobenzamide (Intermediate 853, 0.90 g, 1.35 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.63 g, 1.62 mmol) followed by cesium carbonate (4.14 g, 0.72 mmol). The reaction mixture was purged with argon for 15 min then Pd₂(dba)₃ (0.30 g, 0.32 mmol) followed by Xantphos (0.09 g, 0.18 mmol) were added. The reaction mixture was further purged with argon for 10 min and heated at 100° C. for 3 h. The reaction mixture was concentrated in vacuo. The crude material was purified by combi-flash chromatography, by eluting with 90% ethyl acetate in heptane, to afford tert-butyl 6-(4-(4-(2-(2,6-dioxo-1-((2-(trimethylsilyl)ethoxy)methyl)piperidin-3-yl)-1-oxoisoindolin-4-yl)-3-methylphenethoxy)-2-fluorobenzamido)-2-((R)-1-methylpyrrolidin-2-yl)-1H-pyrrolo[3,2-c]pyridine-1-carboxylate (Intermediate 854, 0.75 g, 59%) as an off-white solid. Mass spec m/z 945.6 [M+H]⁺.

### Step 6

### Example 208: 4-(4-(2-(2,6-dioxopiperidin-3-yl)-1-oxoisoindolin-4-yl)-3-methylphenethoxy)-2-fluoro-N-(2-((R)-1-methylpyrrolidin-2-yl)-1H-pyrrolo[3,2-c]pyridin-6-yl)benzamide

To a solution of tert-butyl 6-(4-(4-(2-(2,6-dioxo-1-((2-(trimethylsilyl)ethoxy)methyl)piperidin-3-yl)-1-oxoisoindolin-4-yl)-3-methylphenethoxy)-2-fluorobenzamido)-2-((R)-1-methylpyrrolidin-2-yl)-1H-pyrrolo[3,2-c]pyridine-1-carboxylate (Intermediate 854, 0.75 g, 0.79 mmol) in acetonitrile (7 mL) was added methanesulfonic acid (0.10 mL, 1.58 mmol) and the reaction mixture was heated at 60° C. for 2 h. The reaction mixture was cooled to room temperature, then N,N′-dimethylethylenediamine (0.19 mL, 1.58 mmol) followed by triethylamine (2.22 mL, 15.9 mmol) were added and the mixture was stirred at room temperature for 3 h. The reaction mixture was concentrated in vacuo and the resultant residue was diluted with water (50 mL). The resultant precipitate was collected by filtration and dried in vacuo. The crude solid was purified by preparative HPLC (Method A) to afford 4-(4-(2-(2,6-dioxopiperidin-3-yl)-1-oxoisoindolin-4-yl)-3-methylphenethoxy)-2-fluoro-N-(2-((R)-1-methylpyrrolidin-2-yl)-1H-pyrrolo[3,2-c]pyridin-6-yl)benzamide (Example 208, 0.16 g, 27%) as a yellow solid. ¹H NMR (400 MHz, DMSO-d₆) δ ppm 1.75-1.98 (m, 5H), 2.10 (s, 3H), 2.16 (s, 3H), 2.20-2.40 (m, 2H), 2.52-2.58 (m, 2H), 2.82-2.90 (m, 1H), 3.06-3.32 (m, 3H), 4.06-4.10 (m, 1H), 4.19-4.23 (m, 1H), 4.33-4.37 (m, 2H), 5.08-5.13 (m, 1H), 6.39 (s, 1H), 6.93 (dd, J=8.80, 2.00 Hz, 1H), 7.00 (dd, J=9.20, 2.40 Hz, 1H), 7.20-7.27 (m, 2H), 7.34 (s, 1H), 7.48 (d, J=4.80 Hz, 1H), 7.61 (t, J=7.60 Hz, 1H), 7.73-7.78 (m, 2H), 8.19 (s, 1H), 8.46 (s, 1H), 9.97 (d, J=5.60 Hz, 1H), 10.92 (s, 1H), 11.37 (s, 1H). Mass spec m/z 715.3 [M+H]⁺.

### Example 209 was Prepared Following Scheme 202

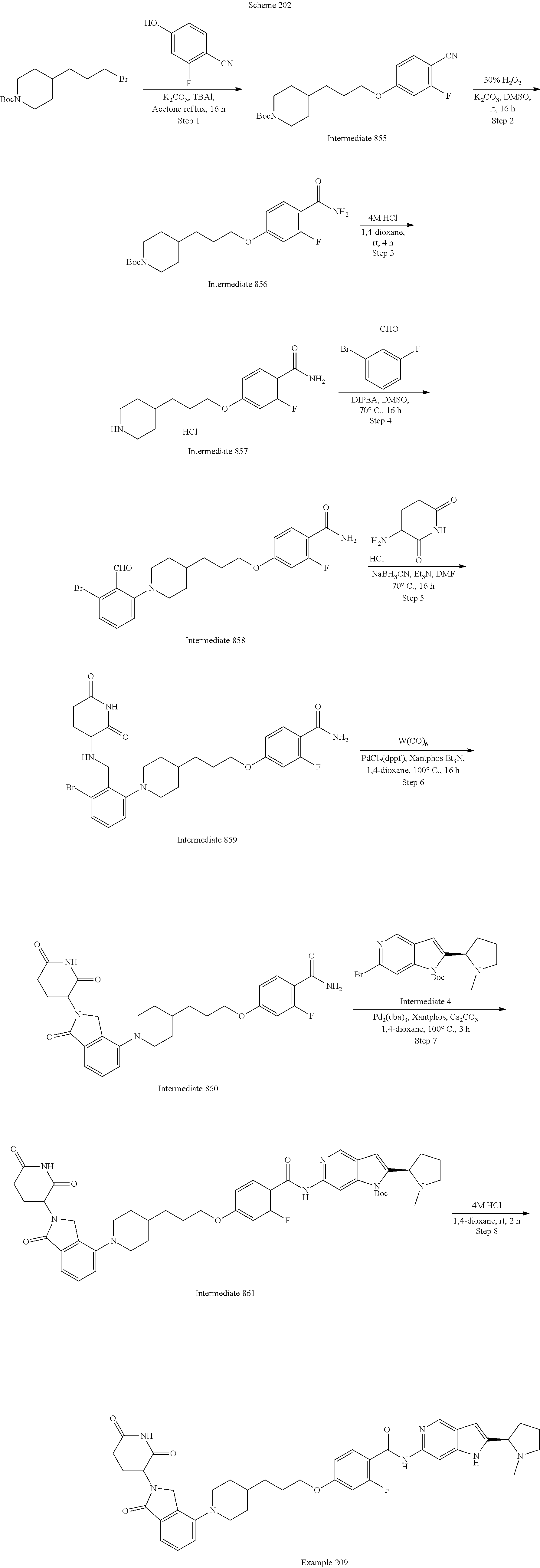

### Step 1

### Intermediate 855: tert-butyl 4-(3-(4-cyano-3-fluorophenoxy)propyl)piperidine-1-carboxylate

To a solution of 2-fluoro-4-hydroxybenzonitrile (CAS No. 82380-18-5, 4.47 g, 32.6 mmol) in acetone (120 mL) was added tert-butyl 4-(3-bromopropyl)piperidine-1-carboxylate (CAS No. 164149-27-3, 9.98 g, 32.6 mmol) followed by potassium carbonate (18.0 g, 130 mmol). Tetrabutylammonium iodide (2.46 g 6.53 mmol) was then added and the reaction mixture was stirred at 65° C. for 16 h. The reaction mixture was then concentrated in vacuo. The crude material was purified by combi-flash chromatography, by eluting with 10% ethyl acetate in heptane, to afford tert-butyl 4-(3-(4-cyano-3-fluorophenoxy)propyl)piperidine-1-carboxylate (Intermediate 855, 10.0 g, 85%) as an off-white solid. ¹H NMR (400 MHz, DMSO-d₆) δ ppm 0.89-1.01 (m, 2H), 1.28-1.47 (m, 12H), 1.60-1.68 (m, 2H), 1.69-1.78 (m, 2H), 2.65 (br s, 2H), 3.87-3.95 (m, 2H), 4.03-4.09 (m, 2H) 6.92-6.96 (m, 1H), 7.09-7.16 (m, 1H), 7.78-7.82 (m, 1H).

### Step 2

### Intermediate 856: tert-butyl 4-(3-(4-carbamoyl-3-fluorophenoxy)propyl)piperidine-1-carboxylate

To a cooled (0° C.) solution of tert-butyl 4-(3-(4-cyano-3-fluorophenoxy)propyl)piperidine-1-carboxylate (Intermediate 855, 9.0 g, 24.8 mmol) in dimethyl sulfoxide (90 mL) was added potassium carbonate (10.3 g, 74.5 mmol) followed by 30% hydrogen peroxide (30 mL, 385 mmol) and the reaction mixture was stirred at room temperature for 16 h. The reaction mixture was then quenched with ice cold water (300 mL). The resultant precipitate was collected by filtration and dried in vacuo to afford tert-butyl 4-(3-(4-carbamoyl-3-fluorophenoxy)propyl)piperidine-1-carboxylate (Intermediate 856, 7.5 g, 80%) as an off-white solid, which was used for the next step without further purification. ¹H NMR (400 MHz, DMSO-d₆) δ ppm 0.89-1.01 (m, 2H), 1.29-1.49 (m, 12H), 1.60-1.74 (m, 4H), 2.59-2.64 (m, 2H), 3.89-4.04 (m, 4H), 6.79-6.87 (m, 2H), 7.39-7.47 (m, 2H), 7.62-7.69 (m, 1H). Mass spec m/z 281.1 [M−100+H]⁺.

### Step 3

### Intermediate 857: 2-fluoro-4-(3-(piperidin-4-yl)propoxy)benzamide hydrochloride

To a solution of tert-butyl 4-(3-(4-carbamoyl-3-fluorophenoxy)propyl)piperidine-1-carboxylate (Intermediate 856, 12 g, 31.6 mmol) in dichloromethane (100 mL) was added 4M hydrochloric acid in 1,4-dioxane (100 mL) and the reaction mixture was stirred at room temperature for 4 h. The reaction mixture was then concentrated in vacuo to afford 2-fluoro-4-(3-(piperidin-4-yl)propoxy)benzamide hydrochloride (Intermediate 857, 9.0 g) as an off-white solid, which was used for the next step without further purification. ¹H NMR (401 MHz, DMSO-d₆) δ ppm 1.28-1.38 (m, 4H), 1.53-1.60 (m, 1H), 1.68-1.82 (m, 4H), 2.74-2.85 (m, 2H), 3.17-3.23 (m, 2H), 4.00-4.05 (m, 2H), 5.93 (br s, 2H), 6.80-6.87 (m, 2H), 7.42 (br s, 2H), 7.66 (t, J=8.80 Hz, 1H). Mass spec m/z 281.1 [M+H]⁺.

### Step 4

### Intermediate 858: 4-(3-(1-(3-bromo-2-formylphenyl)piperidin-4-yl)propoxy)-2-fluorobenzamide

To a solution of 2-fluoro-4-(3-(piperidin-4-yl)propoxy)benzamide hydrochloride (Intermediate 857, 6.0 g, 18.9 mmol) in dimethyl sulfoxide (50 mL) was added N,N-diisopropylethylamine (13.2 mL, 75.8 mmol) followed by 2-bromo-6-fluorobenzaldehyde (CAS No. 360575-28-6, 3.84 g, 18.9 mmol) and the reaction mixture was heated to 70° C. for 16 h. The reaction mixture was then quenched with water (250 mL) and extracted with ethyl acetate (2×125 mL). The combined organic layers were dried over Na₂SO₄, filtered and concentrated in vacuo. The crude material was purified by combi-flash chromatography, by eluting with 30% ethyl acetate in heptane, to afford 4-(3-(1-(3-bromo-2-formylphenyl)piperidin-4-yl)propoxy)-2-fluorobenzamide (Intermediate 858, 6.69 g, 76%) as a pale yellow solid. ¹H NMR (401 MHz, DMSO-d₆) δ ppm 1.18-1.34 (m, 5H), 1.62-1.68 (m, 4H), 2.67-2.77 (m, 2H), 3.02-3.08 (m, 2H), 3.88-3.94 (m, 2H), 6.69-6.77 (m, 2H), 7.09 (d, J=8.40 Hz, 1H), 7.18 (d, J=7.60 Hz, 1H), 7.27-7.34 (m, 3H), 7.55 (t, J=8.40 Hz, 1H), 9.91 (s, 1H). Mass spec m/z 465.1 [M+H+2]+.

### Step 5

### Intermediate 859: 4-(3-(1-(3-bromo-2-(((2,6-dioxopiperidin-3-yl)amino)methyl)phenyl)piperidin-4-yl)propoxy)-2-fluorobenzamide

To a solution of 4-(3-(1-(3-bromo-2-formylphenyl)piperidin-4-yl)propoxy)-2-fluorobenzamide (Intermediate 858, 6.69 g, 14.4 mmol) in acetonitrile (70 mL) was added 3-aminopiperidine-2,6-dione hydrochloride (CAS No. 24666-56-6, 2.85 g, 17.3 mmol) followed by triethylamine (6.02 mL, 43.2 mmol) and the reaction mixture was stirred at room temperature for 2 h. Sodium cyanoborohydride (2.72 g, 43.3 mmol) was then added and the reaction mixture was heated at 70° C. for 16 h. The reaction mixture was quenched with water (200 mL) and extracted with ethyl acetate (3×100 mL). The combined organic layers were dried over Na₂SO₄, filtered, and concentrated in vacuo. The crude material was purified by combi-flash chromatography, by eluting with 10% MeOH in DCM, to afford 4-(3-(1-(3-bromo-2-(((2,6-dioxopiperidin-3-yl)amino)methyl)phenyl)piperidin-4-yl)propoxy)-2-fluorobenzamide (Intermediate 859, 4.0 g, 48%) as a green semi-solid. ¹H NMR (401 MHz, DMSO-d₆) δ ppm 1.19-1.43 (m, 6H), 1.69-1.78 (m, 6H), 2.24-2.34 (m, 2H), 2.54-2.57 (m, 1H), 2.70-2.77 (m, 1H), 3.05-3.13 (m, 1H), 3.21-3.27 (m, 2H), 3.87-3.97 (m, 2H), 4.02-4.07 (m, 2H), 6.83-6.89 (m, 2H), 7.13-7.20 (m, 2H), 7.30-7.45 (m, 1H), 7.40-7.46 (m, 2H), 7.64-7.69 (m, 1H), 10.78 (s, 1H). Mass spec m/z 577.3 [M+H]⁺.

### Step 6

### Intermediate 860: 4-(3-(1-(2-(2,6-dioxopiperidin-3-yl)-1-oxoisoindolin-4-yl)piperidin-4-yl)propoxy)-2-fluorobenzamide

To a solution of 4-(3-(1-(3-bromo-2-(((2,6-dioxopiperidin-3-yl)amino)methyl)phenyl)piperidin-4-yl)propoxy)-2-fluorobenzamide (Intermediate 859, 3.0 g, 5.21 mmol) in 1,4-dioxane (40 mL) was added triethylamine (2.18 mL, 15.6 mmol). The reaction mixture was purged with argon gas for 15 min then Xantphos (0.60 g, 1.04 mmol), PdCl₂(dppf) (0.76 g, 1.04 mmol) and tungsten hexacarbonyl (0.92 g, 2.61 mmol) were added. The reaction mixture was further purged with argon gas for 10 min and heated at 100° C. for 16 h. The reaction mixture was then filtered through a celite bed and the filter pad was washed with ethyl acetate (200 mL). The filtrate was concentrated in vacuo and the crude material purified by combi-flash chromatography, by eluting with 100% ethyl acetate, to afford 4-(3-(1-(2-(2,6-dioxopiperidin-3-yl)-1-oxoisoindolin-4-yl)piperidin-4-yl)propoxy)-2-fluorobenzamide (Intermediate 860, 1.5 g, 56%) as a brown solid. ¹H NMR (401 MHz, DMSO-d₆) δ ppm 1.22-1.44 (m, 6H), 1.74-1.82 (m, 4H), 1.97-2.02 (m, 1H), 2.54-2.58 (m, 1H), 2.67-2.76 (m, 2H), 2.85-2.94 (m, 1H), 3.34-3.42 (m, 2H), 4.01-4.07 (m, 2H), 4.26-4.31 (m, 1H), 4.40-4.45 (m, 1H), 5.08-5.13 (m, 1H), 6.81-6.88 (m, 2H), 7.16 (d, J=8.00 Hz, 1H), 7.29 (d, J=7.60 Hz, 1H), 7.38-7.47 (m, 3H), 7.66-7.69 (t, J=8.80 Hz, 1H), 10.96 (s, 1H). Mass spec m/z 523.1 [M+H]⁺.

### Step 7

### Intermediate 861: tert-butyl 6-(4-(3-(1-(2-(2,6-dioxopiperidin-3-yl)-1-oxoisoindolin-4-yl)piperidin-4-yl)propoxy)-2-fluorobenzamido)-2-((R)-1-methylpyrrolidin-2-yl)-1H-pyrrolo[3,2-c]pyridine-1-carboxylate

To a solution of 4-(3-(1-(2-(2,6-dioxopiperidin-3-yl)-1-oxoisoindolin-4-yl)piperidin-4-yl)propoxy)-2-fluorobenzamide (Intermediate 860, 0.9 g, 1.71 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 0.72 g, 1.89 mmol) followed by cesium carbonate (1.71 g, 5.13 mmol). The reaction mixture was purged with argon for 15 min then Pd₂(dba)₃ (0.39 g, 0.42 mmol) and Xantphos (0.40 g, 0.68 mmol) were added. The reaction mixture was purged with argon for another 10 min and stirred at 100° C. for 3 h. The reaction mixture was then filtered through a celite bed, the filter pad washed with ethyl acetate (100 mL) and filtrate concentrated in vacuo. The crude material was purified by combi-flash chromatography, by eluting with 90% ethyl acetate in heptane, to afford tert-butyl 6-(4-(3-(1-(2-(2,6-dioxopiperidin-3-yl)-1-oxoisoindolin-4-yl)piperidin-4-yl)propoxy)-2-fluorobenzamido)-2-((R)-1-methylpyrrolidin-2-yl)-1H-pyrrolo[3,2-c]pyridine-1-carboxylate (Intermediate 861, 0.56 g, 40%) as an off-white solid. ¹H NMR (400 MHz, DMSO-d₆) δ ppm 1.20-1.49 (m, 7H), 1.56-1.82 (m, 16H), 2.30-2.40 (m, 6H), 2.52-2.60 (m, 1H), 2.67-2.79 (m, 2H), 2.89-2.99 (m, 1H), 3.11-3.14 (m, 1H), 3.32-3.38 (m, 2H), 3.89-3.93 (m, 1H), 4.08-4.13 (m, 1H), 4.26-4.32 (m, 1H), 4.41-4.47 (m, 1H), 5.09-5.13 (m, 1H), 6.74 (s, 1H), 6.88-6.97 (m, 2H), 7.16-7.40 (d, J=8.00 Hz, 1H), 7.29 (d, J=7.20 Hz, 1H), 7.35-7.44 (m, 1H), 7.73 (t, J=8.40 Hz, 1H), 8.55 (s, 1H), 8.92 (s, 1H), 10.26 (d, J=4.80 Hz, 1H), 10.98 (s, 1H). Mass spec m/z 822.2 [M+H]⁺.

### Step 8

### Example 209: 4-(3-(1-(2-(2,6-dioxopiperidin-3-yl)-1-oxoisoindolin-4-yl)piperidin-4-yl)propoxy)-2-fluoro-N-(2-((R)-1-methylpyrrolidin-2-yl)-1H-pyrrolo[3,2-c]pyridin-6-yl)benzamide

To a solution of tert-butyl 6-(4-(3-(1-(2-(2,6-dioxopiperidin-3-yl)-1-oxoisoindolin-4-yl)piperidin-4-yl)propoxy)-2-fluorobenzamido)-2-((R)-1-methylpyrrolidin-2-yl)-1H-pyrrolo[3,2-c]pyridine-1-carboxylate (Intermediate 861, 0.56 g, 0.68 mmol) in dichloromethane (5.0 mL) was added 4M hydrochloric acid in 1,4-dioxane (3.0 mL) and the reaction mixture was stirred at room temperature for 2 h. The reaction mixture was then concentrated in vacuo and the crude material purified by preparative HPLC (Method A) to afford 4-(3-(1-(2-(2,6-dioxopiperidin-3-yl)-1-oxoisoindolin-4-yl)piperidin-4-yl)propoxy)-2-fluoro-N-(2-((R)-1-methylpyrrolidin-2-yl)-1H-pyrrolo[3,2-c]pyridin-6-yl)benzamide (Example 209, 0.051 g, 10%) as an off-white solid. ¹H NMR (400 MHz, DMSO-d₆) δ ppm 1.24-1.52 (m, 5H), 1.77-2.02 (m, 9H), 2.11-2.19 (m, 4H), 2.22-2.28 (m, 1H), 2.52-2.60 (m, 1H), 2.67-2.78 (m, 2H), 2.88-2.94 (m, 1H), 3.11-3.16 (m, 1H), 3.27-3.29 (m, 1H), 3.34-3.43 (m, 2H), 4.07-4.10 (m, 2H), 4.27-4.31 (m, 1H), 4.41-4.45 (m, 1H), 5.46-5.14 (m, 1H), 6.39 (s, 1H), 6.89-6.97 (m, 2H), 7.16 (d, J=8.00 Hz, 1H), 7.29 (d, J=7.80 Hz, 1H), 7.43 (t, J=8.40 Hz, 2H), 7.75 (t, J=8.80 Hz, 1H), 8.46 (s, 1H), 9.95 (d, J=5.60 Hz, 1H), 10.98 (s, 1H), 11.37 (s, 1H). Mass spec m/z 722.1 [M+H]⁺.

### Example 210 was Prepared Following Scheme 203

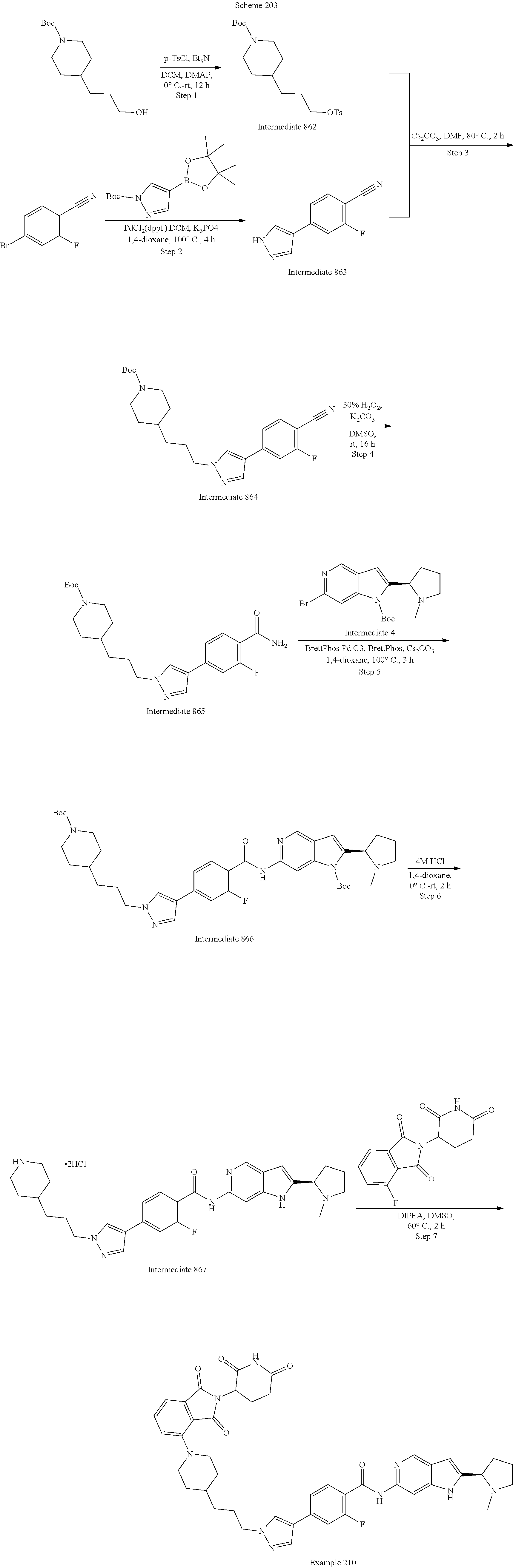

### Step 1

### Intermediate 862: tert-butyl 4-(3-(tosyloxy)propyl)piperidine-1-carboxylate

To a cooled (0° C.) solution of tert-butyl 4-(3-hydroxypropyl)piperidine-1-carboxylate (CAS No. 156185-63-6, 20 g, 82.2 mmol) in dichloromethane (200 mL) was added triethylamine (61 mL, 411 mmol) followed by N,N-dimethylpyridin-4-amine (1.05 g, 8.22 mmol) and p-toluenesulfonyl chloride (23.5 g, 123 mmol). The reaction mixture was stirred at room temperature for 12 h. After completion, water (250 mL) was added and the mixture was extracted with ethyl acetate (2×250 mL). The combined organic layers were dried over Na₂SO₄, filtered and concentrated in vacuo. The crude material was purified by combi-flash chromatography, by eluting with 50% ethyl acetate in heptane, to afford tert-butyl 4-(3-(tosyloxy)propyl)piperidine-1-carboxylate (Intermediate 862, 34.71 g, 80%) as an off-white solid. ¹H NMR (400 MHz, DMSO-d₆) δ ppm 0.78-0.92 (m, 2H), 1.05-1.3 (m, 3H), 1.37 (s, 9H), 1.45-1.6 (m, 4H), 2.4 (s, 3H), 2.5-2,7 (m, 2H), 3.8-3.92 (m, 2H), 3.99 (t, J=6 Hz, 2H), 7.48 (t, J=8 Hz, 2H), 7.78 (t, J=8 Hz, 2H). Mass spec m/z 342.1 [M−56+H]⁺.

### Step 2

### Intermediate 863: 2-fluoro-4-(1H-pyrazol-4-yl)benzonitrile

To a solution of 4-bromo-2-fluorobenzonitrile (CAS No. 105942-08-3, 5.0 g, 25.0 mmol) in 1,4-dioxane (50 mL), water (10 mL) was added tert-butyl 4-(4,4,5,5-tetramethyl-1,3,2-dioxaborolan-2-yl)-1H-pyrazole-1-carboxylate (CAS No. 552846-17-0, 8.82 g, 30.0 mmol) and tripotassium phosphate (16.4 g, 75.0 mmol). The reaction mixture was purged with argon for 15 min then PdCl₂(dppf) (1.92 g, 2.49 mmol) was added. The reaction mixture was further purged with argon for 10 min and heated at 100° C. for 4 h. The reaction mixture was then diluted water (100 mL) and extracted with ethyl acetate (2×100 mL). The combined organic layers were dried over anhydrous Na₂SO₄, filtered and concentrated in vacuo. The crude material was purified by combi-flash chromatography, by eluting with 50% ethyl acetate in heptane, to afford 2-fluoro-4-(1H-pyrazol-4-yl)benzonitrile (Intermediate 863, 3.0 g, 64%) as an off-white solid. ¹H NMR (400 MHz, DMSO-d₆) δ ppm 7.85 (d, J=7.2 Hz, 1H), 7.96 (t, J=7.2 Hz, 1H), 8.04 (d, J=11.80 Hz, 1H), 8.47 (s, 1H), 9.05 (s, 1H). Mass spec m/z 186.0 [M−H]⁻. Step 3

### Intermediate 864: tert-butyl 4-(3-(4-(4-cyano-3-fluorophenyl)-1H-pyrazol-1-yl)propyl)piperidine-1-carboxylate

To a solution of 2-fluoro-4-(1H-pyrazol-4-yl)benzonitrile (Intermediate 863, 2.50 g, 13.0 mmol) in dimethylformamide (25 mL) was added cesium carbonate (13.0 g, 40.0 mmol) and tert-butyl 4-(3-(tosyloxy)propyl)piperidine-1-carboxylate (Intermediate 862, 6.40 g, 16.0 mmol) and the reaction mixture was heated at 80° C. for 2 h. The reaction mixture was then diluted with water (50 mL) and extracted with ethyl acetate (2×100 mL). The combined organic layers were dried over anhydrous Na₂SO₄, filtered and concentrated in vacuo. The crude material was purified by combi-flash chromatography, by eluting with 30% ethyl acetate in heptane, to afford tert-butyl 4-(3-(4-(4-cyano-3-fluorophenyl)-1H-pyrazol-1-yl)propyl)piperidine-1-carboxylate (Intermediate 864, 2.50 g, 45%) as a white solid. ¹H NMR (400 MHz, DMSO-d₆) δ ppm 0.87-0.93 (m, 2H), 1.13-1.20 (m, 2H), 1.31-1.39 (m, 11H), 1.55-1.62 (m, 3H), 1.76-1.84 (m, 2H), 2.58-2.65 (m, 2H), 3.85-3.93 (m, 2H), 7.58 (d, J=8.00 Hz, 1H), 7.75 (d, J=12.2 Hz, 1H), 7.85 (d, J=7.60 Hz, 1H), 8.07 (s, 1H), 8.43 (s, 1H). Mass spec m/z 413.3 [M+H]⁺.

### Step 4

### Intermediate 865: tert-butyl 4-(3-(4-(4-carbamoyl-3-fluorophenyl)-1H-pyrazol-1-yl)propyl)piperidine-1-carboxylate

To a cooled (0° C.) solution of tert-butyl 4-(3-(4-(4-cyano-3-fluorophenyl)-1H-pyrazol-1-yl)propyl)piperidine-1-carboxylate (Intermediate 864, 3.0 g, 7.27 mmol) in dimethyl sulfoxide (30 mL) was added potassium carbonate (3.01 g, 21.82 mmol) followed by 30% hydrogen peroxide (5.68 mL, 72.7 mmol) and the reaction mixture was stirred at room temperature for 16 h. The reaction mixture was diluted with water (100 mL), the resultant precipitate was collected by filtration and dried in vacuo to afford tert-butyl 4-(3-(4-(4-carbamoyl-3-fluorophenyl)-1H-pyrazol-1-yl)propyl)piperidine-1-carboxylate (Intermediate 865, 2.5 g, 80%) as an off-white solid, which was used for the next step without further purification. ¹H NMR (400 MHz, DMSO-d₆) δ ppm 0.87-0.93 (m, 2H), 1.13-1.21 (m, 2H), 1.34-1.37 (s, 10H), 1.55-1.62 (m, 2H), 1.75-1.84 (m, 2H), 2.52-2.65 (m, 2H), 3.84-3.91 (m, 2H), 4.04-4.10 (m, 2H), 7.43-7.56 (m, 4H), 7.65 (t, J=8.40 Hz, 1H), 7.98 (s, 1H), 8.32 (s, 1H). Mass spec m/z 431.2 [M+H]⁺.

### Step 5

### Intermediate 866: tert-butyl (R)-6-(4-(1-(3-(1-(tert-butoxycarbonyl)piperidin-4-yl)propyl)-1H-pyrazol-4-yl)-2-fluorobenzamido)-2-(1-methylpyrrolidin-2-yl)-1H-pyrrolo[3,2-c]pyridine-1-carboxylate

To a solution of tert-butyl 4-(3-(4-(4-carbamoyl-3-fluorophenyl)-1H-pyrazol-1-yl)propyl)piperidine-1-carboxylate (Intermediate 865, 1.0 g, 2.32 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 1.06 g, 2.78 mmol) followed by cesium carbonate (1.32 g, 6.94 mmol). The reaction mixture was purged with argon for 15 min then BrettPhos Pd G3 (0.32 g, 0.34 mmol) and BrettPhos (0.19 g, 0.34 mmol) were added. The reaction mixture was further purged with argon for 10 min and heated at 100° C. for 3 h. The reaction mixture was then filtered through a celite bed, the filter pad was washed with ethyl acetate (100 mL) and the filtrate concentrated in vacuo. The crude material was purified by combi-flash chromatography, by eluting with 9% MeOH in DCM, to afford tert-butyl (R)-6-(4-(1-(3-(1-(tert-butoxycarbonyl)piperidin-4-yl)propyl)-1H-pyrazol-4-yl)-2-fluorobenzamido)-2-(1-methylpyrrolidin-2-yl)-1H-pyrrolo[3,2-c]pyridine-1-carboxylate (Intermediate 866, 1.2 g, 71%) as an off-white solid. Mass spec m/z 730.4 [M+H]⁺.

### Step 6

### Intermediate 867: (R)-2-fluoro-N-(2-(1-methylpyrrolidin-2-yl)-1H-pyrrolo[3,2-c]pyridin-6-yl)-4-(1-(3-(piperidin-4-yl)propyl)-1H-pyrazol-4-yl)benzamide dihydrochloride

To a cooled (0° C.) solution of tert-butyl (R)-6-(4-(1-(3-(1-(tert-butoxycarbonyl)piperidin-4-yl)propyl)-1H-pyrazol-4-yl)-2-fluorobenzamido)-2-(1-methylpyrrolidin-2-yl)-1H-pyrrolo[3,2-c]pyridine-1-carboxylate (Intermediate 866, 1.2 g, 1.6 mmol) in 1,4-dioxane (5 mL) was added 4M hydrochloric acid in 1,4-dioxane (10 mL) and the reaction mixture was stirred at room temperature for 2 h. The reaction mixture was then concentrated in vacuo and the residue triturated with n-pentane (20 mL) to afford (R)-2-fluoro-N-(2-(1-methylpyrrolidin-2-yl)-1H-pyrrolo[3,2-c]pyridin-6-yl)-4-(1-(3-(piperidin-4-yl)propyl)-1H-pyrazol-4-yl)benzamide dihydrochloride (Intermediate 867, 0.8 g, 86%) as an off-white solid. Mass spec m/z 530.3 [M+H]⁺.

### Step 7

### Example 210: 4-(1-(3-(1-(2-(2,6-dioxopiperidin-3-yl)-1,3-dioxoisoindolin-4-yl)piperidin-4-yl)propyl)-1H-pyrazol-4-yl)-2-fluoro-N-(2-((R)-1-methylpyrrolidin-2-yl)-1H-pyrrolo[3,2-c]pyridin-6-yl)benzamide

To a solution of (R)-2-fluoro-N-(2-(1-methylpyrrolidin-2-yl)-1H-pyrrolo[3,2-c]pyridin-6-yl)-4-(1-(3-(piperidin-4-yl)propyl)-1H-pyrazol-4-yl)benzamide dihydrochloride (Intermediate 867, 0.20 g, 0.35 mmol) and 2-(2,6-dioxopiperidin-3-yl)-4-fluoroisoindoline-1,3-dione (CAS No. 835616-60-9, 0.10 g, 0.38 mmol) in dimethyl sulfoxide (2 mL) was added N,N-diisopropylethylamine (0.24 mL, 1.41 mmol) and the reaction mixture was heated at 60° C. for 2 h. The reaction mixture was quenched with ice cold water (100 mL), the resultant precipitate was collected by filtration and dried in vacuo. The crude material was purified by preparative HPLC (Method A) to afford 4-(1-(3-(1-(2-(2,6-dioxopiperidin-3-yl)-1,3-dioxoisoindolin-4-yl)piperidin-4-yl)propyl)-1H-pyrazol-4-yl)-2-fluoro-N-(2-((R)-1-methylpyrrolidin-2-yl)-1H-pyrrolo[3,2-c]pyridin-6-yl)benzamide (Example 210, 0.02 g, 7%) as an off-white solid. ¹H NMR (400 MHz, DMSO-d₆) δ ppm 1.22-1.38 (m, 4H), 1.40-1.49 (m, 1H), 1.75-1.95 (s, 7H), 1.99-2.08 (m, 1H), 2.11-2.20 (m, 4H), 2.22-2.29 (m, 2H), 2.53-2.61 (m, 2H), 2.81-2.88 (m, 3H), 3.13-3.16 (m, 1H), 3.66-3.72 (m, 2H), 4.10-4.18 (m, 2H), 5.04-5.12 (m, 1H), 6.39 (s, 1H), 7.30-7.34 (m, 2H), 7.53-7.61 (m, 2H), 7.64-7.69 (m, 1H), 7.76 (t, J=8.00 Hz, 1H), 8.05 (s, 1H), 8.21 (s, 1H), 8.41 (s, 1H), 8.47 (s, 1H), 10.17 (s, 1H), 11.07 (br s, 1H), 11.39 (s, 1H). Mass spec m/z 786.3 [M+H]⁺.

### Example 211 was Prepared Following Scheme 204

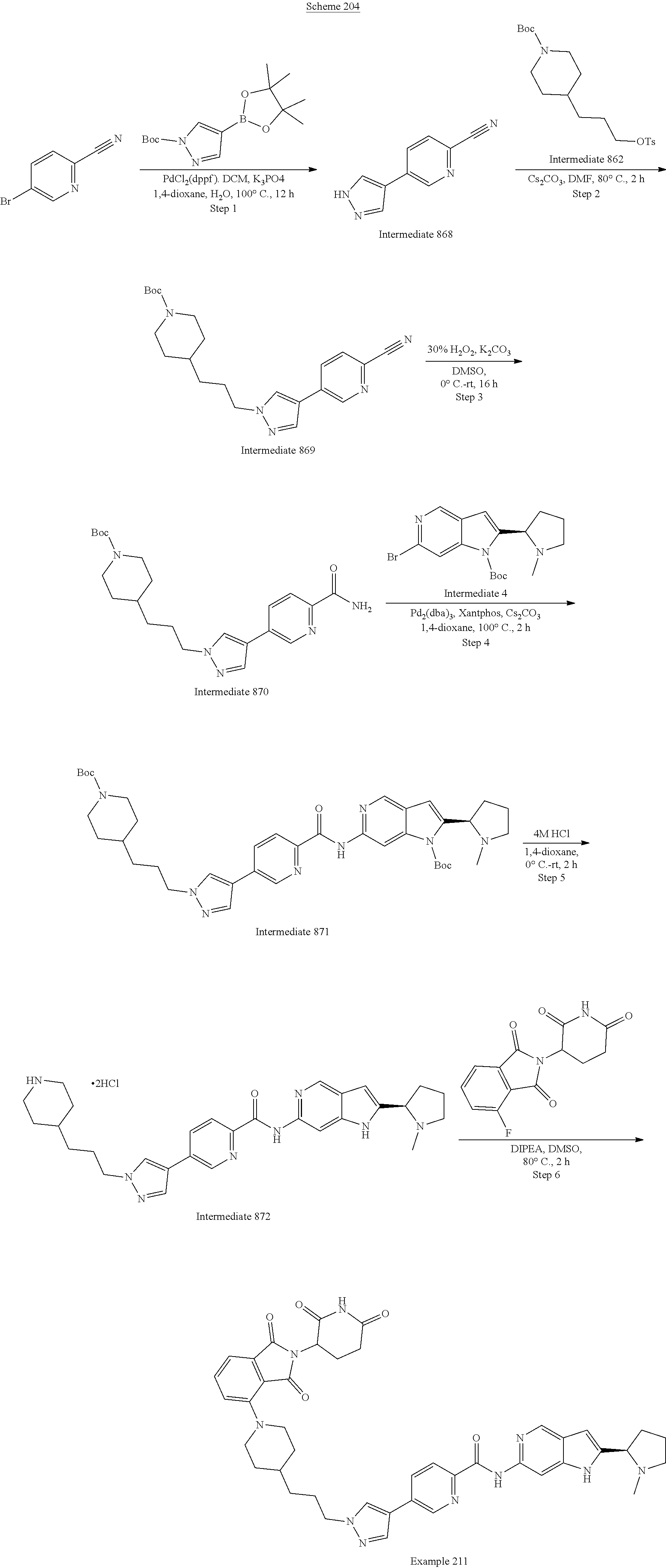

### Step 1

### Intermediate 868: 5-(1H-pyrazol-4-yl)picolinonitrile

To a solution of 5-bromopicolinonitrile (CAS No. 97483-77-7, 5.0 g, 27 mmol) in 1,4-dioxane (30 mL) and water (10 mL) was added tert-butyl 4-(4,4,5,5-tetramethyl-1,3,2-dioxaborolan-2-yl)-1H-pyrazole-1-carboxylate (CAS No. 552846-17-0, 9.6 g, 33 mmol) followed by tripotassium phosphate (18 g, 82 mmol). The reaction mixture was purged with argon for 15 min then PdCl₂(dppf) (2.1 g, 2.7 mmol) was added. The reaction mixture was purged with argon for another 10 min and heated at 100° C. for 12 h. The reaction mixture was then quenched with ice cold water (100 mL) and extracted with ethyl acetate (2×100 mL). The combined organic layers were dried over anhydrous Na₂SO₄, filtered and concentrated in vacuo. The crude material was purified by combi-flash chromatography, by eluting with 80% ethyl acetate in heptane, to afford 5-(1H-pyrazol-4-yl)picolinonitrile (Intermediate 868, 2.5 g, 34%) as brown solid. ¹H NMR (400 MHz, DMSO-d₆) δ ppm 8.00 (d, J=8.25 Hz, 1H), 8.18 (br s, 1H), 8.23-8.25 (m, 1H), 8.51 (s, 1H), 9.07 (s, 1H), 13.25 (br s, 1H). Mass spec m/z 171.1 [M+H]⁺.

### Step 2

### Intermediate 869: tert-butyl 4-(3-(4-(6-cyanopyridin-3-yl)-1H-pyrazol-1-yl)propyl)piperidine-1-carboxylate

To a solution of 5-(1H-pyrazol-4-yl)picolinonitrile (Intermediate 868, 2.5 g, 14.7 mmol) in dimethylformamide (10 mL) was added cesium carbonate (14.4 g, 44.1 mmol) and the reaction mixture was stirred at room temperature for 10 min. A solution of tert-butyl 4-(3-(tosyloxy)propyl)piperidine-1-carboxylate (Intermediate 862, 7.0 g, 17.6 mmol) in dimethylformamide (10 mL) was then added and the reaction mixture heated at 80° C. for 2 h. The reaction was then quenched with ice cool water (100 mL) and extracted with ethyl acetate (2×100 mL). The combined organic layers were dried over anhydrous Na₂SO₄, filtered and concentrated in vacuo. The crude material was purified by combi-flash chromatography, by eluting with 30% ethyl acetate in heptane, to afford tert-butyl 4-(3-(4-(6-cyanopyridin-3-yl)-1H-pyrazol-1-yl)propyl)piperidine-1-carboxylate (Intermediate 869, 3.0 g, 52%) as a white solid. ¹H NMR (400 MHz, DMSO-d₆) δ ppm 0.87-0.97 (m, 2H), 1.15-1.21 (m, 2H), 1.31-1.35 (m, 2H), 1.37 (s, 9H), 1.59-1.62 (m, 2H), 1.79-1.86 (m, 2H), 2.58-2.69 (m, 1H), 2.73-2.89 (m, 2H), 3.88-3.92 (m, 2H), 7.95 (s, 1H), 7.99-8.01 (m, 1H), 8.14 (s, 1H), 8.18-8.21 (m, 1H), 8.50 (s, 1H). Mass spec m/z 396.1 [M−100+H]⁺.

### Step 3

### Intermediate 870: tert-butyl 4-(3-(4-(6-carbamoylpyridin-3-yl)-1H-pyrazol-1-yl)propyl)piperidine-1-carboxylate

To a cooled (0° C.) solution of tert-butyl 4-(3-(4-(6-cyanopyridin-3-yl)-1H-pyrazol-1-yl)propyl)piperidine-1-carboxylate (Intermediate 869, 2.5 g, 6.32 mmol) in dimethyl sulfoxide (10.0 mL) was added potassium carbonate (2.18 g, 15.8 mmol) followed by 30% hydrogen peroxide (4.94 mL, 63.2 mmol) and the reaction mixture was stirred at room temperature for 16 h. The reaction was then quenched with ice cold water (300 mL). The resultant precipitate was collected by filtration and dried in vacuo to afford tert-butyl 4-(3-(4-(6-carbamoylpyridin-3-yl)-1H-pyrazol-1-yl)propyl)piperidine-1-carboxylate (Intermediate 870, 2.2 g, 84%) as an off-white solid, which was used for the next step without further purification. ¹H NMR (400 MHz, DMSO-d₆) δ ppm 0.91-0.93 (m, 2H), 1.17-1.21 (m, 2H), 1.37 (s, 9H), 1.59-1.62 (m, 2H), 1.55-1.62 (m, 2H), 1.81-1.85 (m, 2H), 2.62-2.69 (m, 1H), 3.88-3.91 (m, 2H), 4.10-4.14 (m, 2H), 7.57 (br s, 1H), 7.98-8.13 (m, 4H), 8.42 (br s, 1H), 8.86 (s, 1H). Mass spec m/z 414.2 [M+H]⁺.

### Step 4

### Intermediate 871: tert-butyl (R)-6-(5-(1-(3-(1-(tert-butoxycarbonyl)piperidin-4-yl)propyl)-1H-pyrazol-4-yl)picolinamido)-2-(1-methylpyrrolidin-2-yl)-1H-pyrrolo[3,2-c]pyridine-1-carboxylate

To a solution of tert-butyl 4-(3-(4-(6-carbamoylpyridin-3-yl)-1H-pyrazol-1-yl)propyl)piperidine-1-carboxylate (Intermediate 870, 1.0 g, 1.0 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.66 g, 1.7 mmol) followed by cesium carbonate (0.70 g, 2.0 mmol). The reaction mixture was purged with argon for 15 min then Pd₂(dba)₃ (2.50 g, 2.9 mmol) and Xantphos (0.60 g, 1.0 mmol) were added. The reaction mixture was purged with argon for another 10 min and heated at 100° C. for 2 h. After completion, the reaction mixture was filtered through a celite bed, the filter pad was washed with ethyl acetate (100 mL) and the filtrate concentrated in vacuo. The crude material was purified by combi-flash chromatography, by eluting with 5% MeOH in DCM, to afford tert-butyl (R)-6-(5-(1-(3-(1-(tert-butoxycarbonyl)piperidin-4-yl)propyl)-1H-pyrazol-4-yl)picolinamido)-2-(1-methylpyrrolidin-2-yl)-1H-pyrrolo[3,2-c]pyridine-1-carboxylate (Intermediate 871, 1.2 g, 70%) as an off-white solid. Mass spec m/z 713.9 [M+H]⁺.

### Step 5

### Intermediate 872: (R)—N-(2-(1-methylpyrrolidin-2-yl)-1H-pyrrolo[3,2-c]pyridin-6-yl)-5-(1-(3-(piperidin-4-yl)propyl)-1H-pyrazol-4-yl)picolinamide dihydrochloride

To a cooled (0° C.) solution of tert-butyl (R)-6-(5-(1-(3-(1-(tert-butoxycarbonyl)piperidin-4-yl)propyl)-1H-pyrazol-4-yl)picolinamido)-2-(1-methylpyrrolidin-2-yl)-1H-pyrrolo[3,2-c]pyridine-1-carboxylate (Intermediate 871, 0.7 g, 1.0 mmol) in dichloromethane (5.0 mL) was added 4M hydrochloric acid in dioxane (7.0 mL) and the reaction mixture was stirred at room temperature for 2 h. The reaction mixture was then concentrated in vacuo and the residue triturated with n-pentane (20 mL) to afford (R)—N-(2-(1-methylpyrrolidin-2-yl)-1H-pyrrolo[3,2-c]pyridin-6-yl)-5-(1-(3-(piperidin-4-yl)propyl)-1H-pyrazol-4-yl)picolinamide dihydrochloride (Intermediate 872, 0.5 g, 90%) as an off-white solid, which was used for the next step without further purification. ¹H NMR (400 MHz, DMSO-d₆) δ ppm 1.20-1.29 (m, 3H), 1.42-1.52 (m, 1H), 1.75-1.83 (m, 3H), 2.80 (s, 3H), 3.19-3.22 (m, 2H), 3.54-3.60 (m, 6H), 4.14-4.17 (m, 2H), 4.58-4.75 (m, 4H), 7.29-7.35 (m, 2H), 7.67 (dd, J=8.32, 7.19 Hz, 1H), 8.15 (s, 1H), 8.18 (d, J=8.13 Hz, 1H), 8.27 (dd, J=8.19, 2.19 Hz, 1H), 8.29 (s, 1H), 8.51 (d, J=8.33 Hz, 2H), 9.01 (s, 1H), 11.50 (br s, 1H), 13.23 (br s, 1H). Mass spec m/z 513.3 [M+H]⁺.

### Step 6

### Example 211: 5-(1-(3-(1-(2-(2,6-dioxopiperidin-3-yl)-1,3-dioxoisoindolin-4-yl)piperidin-4-yl)propyl)-1H-pyrazol-4-yl)-N-(2-((R)-1-methylpyrrolidin-2-yl)-1H-pyrrolo[3,2-c]pyridin-6-yl)picolinamide

To a solution of (R)—N-(2-(1-methylpyrrolidin-2-yl)-1H-pyrrolo[3,2-c]pyridin-6-yl)-5-(1-(3-(piperidin-4-yl)propyl)-1H-pyrazol-4-yl)picolinamide dihydrochloride (Intermediate 872, 0.6 g, 1.0 mmol) and 2-(2,6-dioxopiperidin-3-yl)-4-fluoroisoindoline-1,3-dione (CAS No. 835616-60-9, 0.2 g, 0.9 mmol) in dimethyl sulfoxide (1 mL) were added N,N-diisopropylethylamine (1.0 mL, 7 mmol). The reaction mixture was heated at 80° C. for 2 h. The reaction mixture was concentrated in vacuo. The residue was diluted with ice cold water (100 mL), the resultant precipitate was collected by filtration, washed with n-pentane (50 mL), and dried in vacuo. The crude material was purified by preparative HPLC (Method B) to afford 5-(1-(3-(1-(2-(2,6-dioxopiperidin-3-yl)-1,3-dioxoisoindolin-4-yl)piperidin-4-yl)propyl)-1H-pyrazol-4-yl)-N-(2-((R)-1-methylpyrrolidin-2-yl)-1H-pyrrolo[3,2-c]pyridin-6-yl)picolinamide (Example 211, 0.042 g, 5%) as a yellow solid. ¹H NMR (400 MHz, DMSO-d₆) δ ppm 1.23-1.38 (m, 4H), 1.42-1.52 (m, 1H), 1.74-1.82 (m, 3H), 1.86-1.95 (m, 4H), 1.98-2.06 (m, 1H), 2.10-2.15 (m, 1H), 2.17 (s, 3H), 2.23-2.30 (m, 1H), 2.54-2.62 (m, 2H), 2.80-2.89 (m, 3H), 3.11-3.18 (m, 1H), 3.32-3.34 (m, 1H), 3.66-3.70 (m, 2H), 4.19-4.20 (m, 2H), 5.05-5.10 (m, 1H), 6.41 (s, 1H), 7.29-7.35 (m, 2H), 7.67 (dd, J=8.32, 7.19 Hz, 1H), 8.15 (s, 1H), 8.18 (d, J=8.13 Hz, 1H), 8.27 (dd, J=8.19, 2.19 Hz, 1H), 8.29 (s, 1H), 8.51 (d, J=8.33 Hz, 2H), 9.01 (s, 1H), 10.26 (br s, 1H), 11.07 (br s, 1H), 11.45 (br s, 1H). Mass spec m/z 769.3 [M+H]⁺.

### Example 212 was Prepared Following Scheme 205

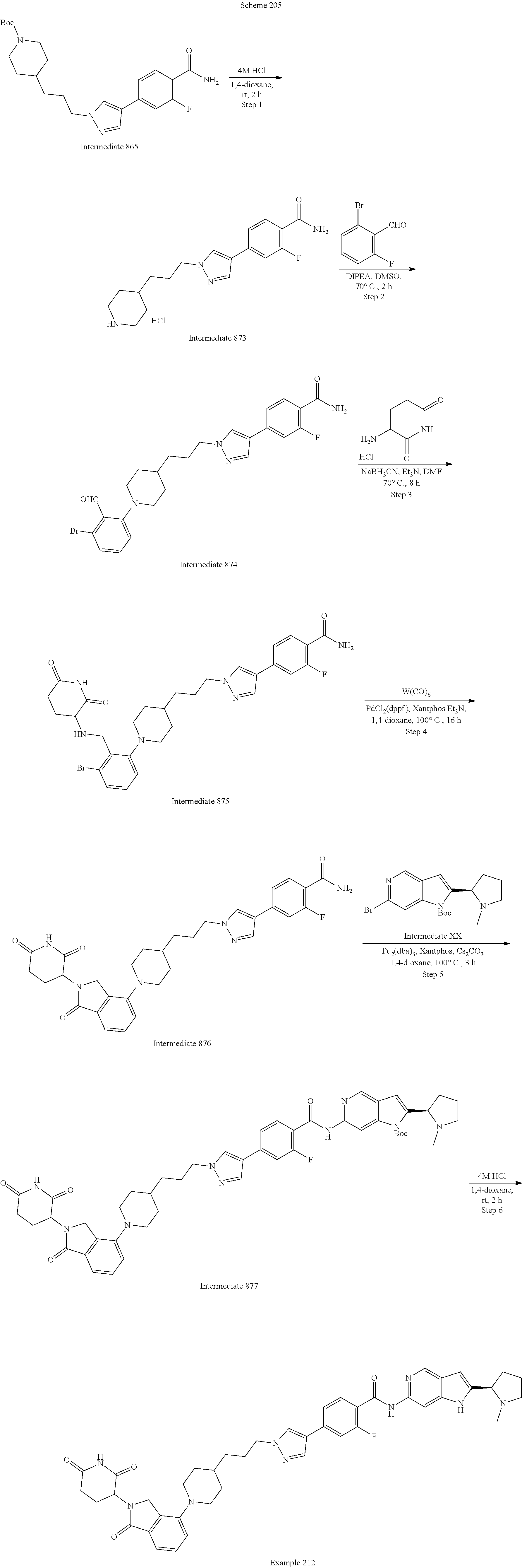

### Step 1

### Intermediate 873: 2-fluoro-4-(1-(3-(piperidin-4-yl)propyl)-1H-pyrazol-4-yl)benzamide hydrochloride

To a solution of tert-butyl 4-(3-(4-(4-carbamoyl-3-fluorophenyl)-1H-pyrazol-1-yl)propyl)piperidine-1-carboxylate (Intermediate 865, 3.5 g, 8.1 mmol) in dichloromethane (35 mL) was added 4M hydrochloric acid in 1,4-dioxane (15 mL) and the reaction mixture was stirred at room temperature for 2 h. The reaction mixture was then concentrated in vacuo to afford 2-fluoro-4-(1-(3-(piperidin-4-yl)propyl)-1H-pyrazol-4-yl)benzamide hydrochloride (Intermediate 873, 2.9 g, 97%) as an off-white solid, which was used for the next step without further purification. ¹H NMR (401 MHz, DMSO-d₆) δ ppm 1.15-1.33 (m, 4H), 1.48-1.53 (s, 1H), 1.59-1.83 (m, 4H), 2.72-2.82 (m, 2H), 3.17-3.20 (m, 2H), 4.09-4.12 (m, 2H), 7.45-7.52 (m, 2H), 7.66 (t, J=7.6 Hz, 1H), 8.00 (s, 1H), 8.35 (s, 1H), 8.81 (bs, 1H), 9.05 (bs, 1H). Mass spec m/z 331.1 [M+H]⁺.

### Step 2

### Intermediate 874: 4-(1-(3-(1-(3-bromo-2-formylphenyl)piperidin-4-yl)propyl)-1H-pyrazol-4-yl)-2-fluorobenzamide

To a solution of 2-fluoro-4-(1-(3-(piperidin-4-yl)propyl)-1H-pyrazol-4-yl)benzamide hydrochloride (Intermediate 873, 2.9 g, 7.9 mmol) in dimethylformamide (70 mL) were added potassium carbonate (3.8 g, 28 mmol) followed by 2-bromo-6-fluorobenzaldehyde (CAS No. 360575-28-6, 1.6 g, 7.9 mmol) and the reaction mixture was heated at 70° C. for 2 h. The reaction mixture was quenched with water (250 mL) and extracted with ethyl acetate (2×125 mL). The combined organic layers were dried over Na₂SO₄, filter and concentrated in vacuo to afford 4-(1-(3-(1-(3-bromo-2-formylphenyl)piperidin-4-yl)propyl)-1H-pyrazol-4-yl)-2-fluorobenzamide (Intermediate 874, 4.0 g) as a pale-yellow solid which was used for the next step without further purification. ¹H NMR (401 MHz, DMSO-d₆) δ ppm 1.22-1.45 (m, 5H), 1.71-1.76 (m, 2H), 1.83-1.89 (m, 2H), 2.81-2.86 (m, 2H), 3.12-3.17 (m, 2H), 4.09-4.15 (m, 2H), 7.20 (d, J=8.40 Hz, 1H), 7.29 (d, J=8.40 Hz, 1H), 7.40 (d, J=8.00 Hz, 1H), 7.46 (m, 4H), 7.67 (t, J=8.0 Hz, 1H), 8.00 (s, 1H), 8.36 (s, 1H), 10.01 (s, 1H). Mass spec m/z 513.0 [M+H]⁺.

### Step 3

### Intermediate 875: 4-(1-(3-(1-(3-bromo-2-(((2,6-dioxopiperidin-3-yl)amino)methyl)phenyl)piperidin-4-yl)propyl)-1H-pyrazol-4-yl)-2-fluorobenzamide

To a solution of 4-(1-(3-(1-(3-bromo-2-formylphenyl)piperidin-4-yl)propyl)-1H-pyrazol-4-yl)-2-fluorobenzamide (Intermediate 874, 3.5 g, 6.8 mmol) in acetonitrile (40 mL) was added 3-aminopiperidine-2,6-dione hydrochloride (CAS No. 24666-56-6, 1.5 g, 8.9 mmol) followed by triethylamine (2.8 mL, 20.4 mmol) and the reaction mixture was stirred at room temperature for 2 h. Sodium cyanoborohydride (1.8 g, 27 mmol) was then added and reaction mixture was heated at 70° C. for 8 h. The reaction mixture was quenched with water (100 ml) and extracted with ethyl acetate (3×100 mL). The combined organic layers were dried over Na₂SO₄, filtered, and concentrated in vacuo. The crude material was purified by combi-flash chromatography, by eluting with 10% MeOH in DCM, to afford 4-(1-(3-(1-(3-bromo-2-(((2,6-dioxopiperidin-3-yl)amino)methyl)phenyl)piperidin-4-yl)propyl)-1H-pyrazol-4-yl)-2-fluorobenzamide (Intermediate 875, 3 g, 70%) as a green semi-solid. ¹H NMR (401 MHz, DMSO-d₆) δ ppm 1.10-1.34 (m, 7H), 1.68-1.88 (m, 6H), 2.70-2.74 (m, 1H), 2.91-3.12 (m, 2H), 3.21-3.30 (m, 2H), 3.81-3.99 (m, 2H), 4.01-4.04 (m, 1H), 4.10-4.14 (m, 2H), 7.11-7.20 (m, 2H), 7.29-7.33 (m, 1H), 7.45-7.58 (m, 4H), 7.64-7.67 (m, 1H), 8.01 (s, 1H), 8.36 (s, 1H), 10.78 (s, 1H). Mass spec m/z 626.6 [M+H]⁺.

### Step 4

### Intermediate 876: 4-(1-(3-(1-(2-(2,6-dioxopiperidin-3-yl)-1-oxoisoindolin-4-yl)piperidin-4-yl)propyl)-1H-pyrazol-4-yl)-2-fluorobenzamide

To a solution of 4-(1-(3-(1-(3-bromo-2-(((2,6-dioxopiperidin-3-yl)amino)methyl)phenyl)piperidin-4-yl)propyl)-1H-pyrazol-4-yl)-2-fluorobenzamide (Intermediate 875, 2 g, 3.20 mmol) in 1,4-dioxane (20 mL) was added triethylamine (1.34 mL, 9.59 mmol). The reaction mixture was purged with argon gas for 15 min then Xantphos (0.38 g, 0.64 mmol) followed by Pd(dppf)Cl₂ (0.49 g, 0.63 mmol) and tungsten hexacarbonyl (0.56 g, 1.6 mmol) were added. The reaction mixture was further purged with argon gas for 10 min and heated at 100° C. for 16 h. The reaction mixture was filtered through a celite bed and the filter pad was washed with ethyl acetate (200 mL). The filtrate was concentrated in vacuo and the crude material purified by combi-flash chromatography, by eluting with 10% MeOH in DCM, to afford 4-(1-(3-(1-(2-(2,6-dioxopiperidin-3-yl)-1-oxoisoindolin-4-yl)piperidin-4-yl)propyl)-1H-pyrazol-4-yl)-2-fluorobenzamide (Intermediate 876, 1.5 g, 82%) as a yellow solid. Mass spec m/z 573.3 [M+H]⁺.

### Step 5

### Intermediate 877: tert-butyl 6-(4-(1-(3-(1-(2-(2,6-dioxopiperidin-3-yl)-1-oxoisoindolin-4-yl)piperidin-4-yl)propyl)-1H-pyrazol-4-yl)-2-fluorobenzamido)-2-((R)-1-methylpyrrolidin-2-yl)-1H-pyrrolo[3,2-c]pyridine-1-carboxylate

To a solution of 4-(1-(3-(1-(2-(2,6-dioxopiperidin-3-yl)-1-oxoisoindolin-4-yl)piperidin-4-yl)propyl)-1H-pyrazol-4-yl)-2-fluorobenzamide (Intermediate 876, 0.70 g, 1.19 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 4, 0.53 g, 1.40 mmol) followed by cesium carbonate (1.19 g, 3.64 mmol). The reaction mixture was purged with argon for 15 min then Pd₂(dba)₃ (0.17 g, 0.18 mmol) and Xantphos (0.105 g, 0.18 mmol) were added. The reaction mixture was purged with argon for another 10 min and heated at 100° C. for 3 h. The reaction mixture was filtered through a celite bed, the filter pad was washed with ethyl acetate (100 mL) and the filtrate concentrated in vacuo. The crude material was purified by combi-flash chromatography, by eluting with 90% ethyl acetate in heptane, to afford tert-butyl 6-(4-(1-(3-(1-(2-(2,6-dioxopiperidin-3-yl)-1-oxoisoindolin-4-yl)piperidin-4-yl)propyl)-1H-pyrazol-4-yl)-2-fluorobenzamido)-2-((R)-1-methylpyrrolidin-2-yl)-1H-pyrrolo[3,2-c]pyridine-1-carboxylate (Intermediate 877, 0.77 g, 77%) as a yellow solid. ¹H NMR (401 MHz, DMSO-d₆) δ ppm 0.83-0.89 (m, 6H), 1.19-1.56 (m, 8H), 1.49-2.01 (m, 14H), 2.31-2.40 (m, 4H), 2.54-2.71 (m, 2H), 2.86-2.91 (m, 1H), 3.12-3.16 (m, 1H), 3.80-3.94 (m, 1H), 4.11-4.15 (m, 2H), 4.25-4.32 (m, 1H), 4.37-4.45 (m, 1H), 5.10-5.14 (m, 1H), 6.65 (s, 1H), 7.13-7.16 (m, 1H), 7.29-7.31 (m, 1H), 7.39-7.43 (m, 1H), 7.51-7.56 (m, 1H), 7.71-7.74 (m, 1H), 8.07 (s, 1H), 8.41 (s, 1H), 8.59 (s, 1H), 8.63 (s, 1H), 10.49 (s, 1H), 10.99 (s, 1H). Mass spec m/z 872.6 [M+H]⁺.

### Step 6

### Example 212: 4-(1-(3-(1-(2-(2,6-dioxopiperidin-3-yl)-1-oxoisoindolin-4-yl)piperidin-4-yl)propyl)-1H-pyrazol-4-yl)-2-fluoro-N-(2-((R)-1-methylpyrrolidin-2-yl)-1H-pyrrolo[3,2-c]pyridin-6-yl)benzamide

To a solution of tert-butyl 6-(4-(1-(3-(1-(2-(2,6-dioxopiperidin-3-yl)-1-oxoisoindolin-4-yl)piperidin-4-yl)propyl)-1H-pyrazol-4-yl)-2-fluorobenzamido)-2-((R)-1-methylpyrrolidin-2-yl)-1H-pyrrolo[3,2-c]pyridine-1-carboxylate (Intermediate 877, 0.7 g, 0.80 mmol) in dichloromethane (7 mL) was added 4M hydrochloric acid in dioxane (5.0 mL) and the reaction mixture was stirred at room temperature for 2 h. The reaction mixture was then concentrated in vacuo. The crude material was purified by preparative HPLC (Method A) to afford 4-(1-(3-(1-(2-(2,6-dioxopiperidin-3-yl)-1-oxoisoindolin-4-yl)piperidin-4-yl)propyl)-1H-pyrazol-4-yl)-2-fluoro-N-(2-((R)-1-methylpyrrolidin-2-yl)-1H-pyrrolo[3,2-c]pyridin-6-yl)benzamide (Example 212, 0.06 g, 10%) as an off-white solid. ¹H NMR (401 MHz, DMSO-d₆) δ ppm 1.20-1.31 (m, 4H), 1.38-1.48 (m, 1H), 1.75-2.01 (m, 8H), 2.11-2.20 (m, 4H), 2.13-2.29 (m, 1H), 2.43-2.48 (m, 4H), 2.53-2.61 (m, 1H), 2.70-2.76 (m, 1H), 2.88-2.95 (m, 1H), 3.13-3.18 (m, 1H), 3.33-3.41 (m, 1H), 4.11-4.17 (m, 2H), 4.25-4.30 (m, 1H), 4.37-4.45 (m, 1H), 5.11-5.13 (m, 1H), 6.39 (s, 1H), 7.15 (d, J=7.60 Hz, 1H), 7.29 (d, J=6.80 Hz, 1H), 7.41 (t, J=7.60 Hz, 1H), 7.53-7.61 (m, 2H), 7.76 (t, J=8.00 Hz, 1H), 8.05 (s, 1H), 8.21 (s, 1H), 8.40 (s, 1H), 8.48 (s, 1H), 10.17 (d, J=4.40 Hz, 1H), 10.97 (s, 1H), 11.39 (s, 1H). Mass spec m/z 772.3 [M+H]⁺.

### Example 213 was Prepared Following Scheme 206

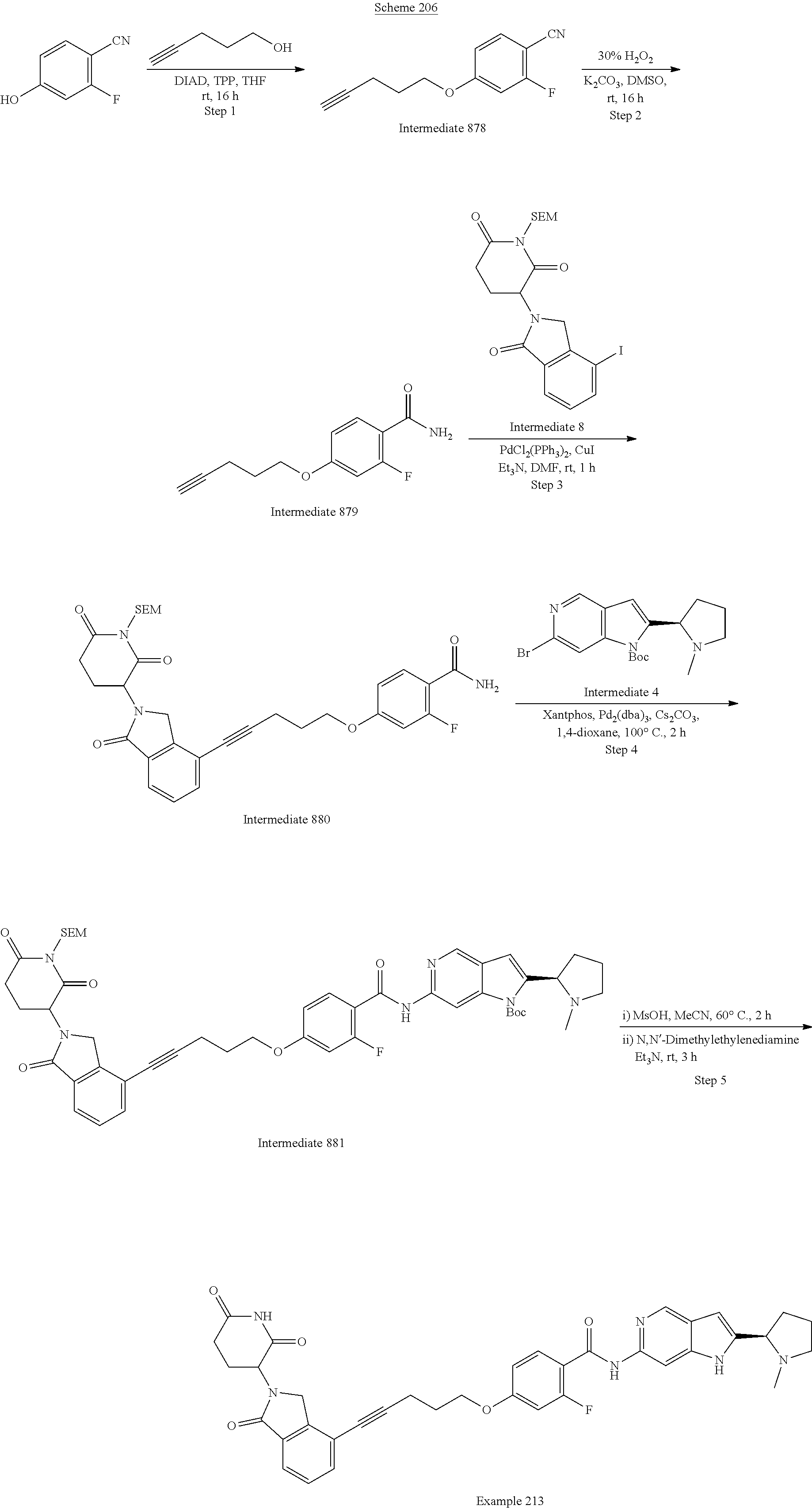

### Step 1

### Intermediate 878: 2-fluoro-4-(pent-4-yn-1-yloxy)benzonitrile

To a solution of 2-fluoro-4-hydroxybenzonitrile (CAS No. 82380-18-5, 10 g, 72.9 mmol) in tetrahydrofuran (120 mL) was added pent-4-yn-1-ol (CAS No. 5390-04-5, 7.97 g, 94.8 mmol) followed by triphenylphosphine (36.1 g, 109 mmol). Diisopropyl azodicarboxylate (30.8 mL 146 mmol) was then added and the reaction mixture was stirred at room temperature for 16 h. The reaction mixture was concentrated in vacuo to afford 2-fluoro-4-(pent-4-yn-1-yloxy)benzonitrile (Intermediate 878, 15.0 g), which was used for next step without further purification. ¹H NMR (400 MHz, DMSO-d₆) δ ppm 1.86-1.93 (m, 2H), 2.29-2.35 (m, 2H), 2.80-3.82 (m, 1H), 4.12-4.18 (m, 2H), 6.97 (dd, J=8.4, 2.0 Hz, 1H), 7.15 (dd, J=11.6, 2.0 Hz, 1H), 7.81 (t, J=8.0 Hz, 1H).

### Step 2

### Intermediate 879: 2-fluoro-4-(pent-4-yn-1-yloxy)benzamide

To a cooled (0° C.) solution of 2-fluoro-4-(pent-4-yn-1-yloxy)benzonitrile (Intermediate 878, 15 g, 73.8 mmol) in dimethyl sulfoxide (30 mL) was added potassium carbonate (20.4 g, 148 mmol) followed by 30% hydrogen peroxide (23.1 mL, 295 mmol) and the reaction mixture was stirred at room temperature for 16 h. The reaction mixture was then quenched with ice cold water (500 mL), the resultant precipitate was collected by filtration and dried in vacuo to afford 2-fluoro-4-(pent-4-yn-1-yloxy)benzamide (Intermediate 879, 10.0 g, 61%) as an off-white solid, which was used for the next step without further purification. ¹H NMR (400 MHz, DMSO-d₆) δ ppm 1.84-1.92 (m, 2H), 2.27-2.35 (m, 2H), 2.80-3.81 (m, 1H), 4.04-4.09 (m, 2H), 6.81-6.88 (m, 2H), 7.35-7.48 (m, 2H), 7.65 (t, J=8.80 Hz, 1H). Mass spec m/z 222.0 [M+H]⁺.

### Step 3

### Intermediate 880: 4-((5-(2-(2,6-dioxo-1-((2-(trimethylsilyl)ethoxy)methyl)piperidin-3-yl)-1-oxoisoindolin-4-yl)pent-4-yn-1-yl)oxy)-2-fluorobenzamide

To a solution of 2-fluoro-4-(pent-4-yn-1-yloxy)benzamide (Intermediate 879, 1.0 g, 4.52 mmol) in dimethylformamide (20 mL) was added 3-(4-iodo-1-oxoisoindolin-2-yl)-1-((2-(trimethylsilyl)ethoxy)methyl)piperidine-2,6-dione (Intermediate 8, 2.26 g, 4.52 mmol) followed by triethylamine (22.8 mL, 163 mmol). The reaction mixture was purged with argon for 15 min then PdCl₂(PPh₃)₂ (0.32 g, 0.45 mmol) was added. The reaction mixture was purged with argon for another 10 min and stirred at room temperature for 1 h. The reaction mixture was then concentrated in vacuo and the crude material was purified by combi-flash chromatography, by eluting with 80% ethyl acetate in heptane, to afford 4-((5-(2-(2,6-dioxo-1-((2-(trimethylsilyl)ethoxy)methyl)piperidin-3-yl)-1-oxoisoindolin-4-yl)pent-4-yn-1-yl)oxy)-2-fluorobenzamide (Intermediate 880, 1.5 g, 56%) as an off-white solid. ¹H NMR (400 MHz, DMSO-d₆) δ ppm −0.03 (s, 9H), 0.76-0.89 (m, 2H), 2.01-2.05 (m, 3H), 2.31-2.42 (m, 1H), 2.62-2.68 (m, 2H), 2.71-2.81 (m, 1H), 3.01-3.11 (m, 1H), 3.46-3.53 (m, 2H), 4.15-4.29 (m, 3H), 4.41-4.49 (m, 1H), 5.01-5.09 (m, 2H), 5.22-5.28 (m, 1H), 6.84-6.94 (m, 2H), 7.40 (br s, 1H), 7.46-7.57 (m, 2H), 7.65-7.73 (m, 3H). Mass spec m/z 592.2 [M−H]⁻.

### Step 4

### Intermediate 881: tert-butyl 6-(4-((5-(2-(2,6-dioxo-1-((2-(trimethylsilyl)ethoxy)methyl)piperidin-3-yl)-1-oxoisoindolin-4-yl)pent-4-yn-1-yl)oxy)-2-fluorobenzamido)-2-((R)-1-methylpyrrolidin-2-yl)-1H-pyrrolo[3,2-c]pyridine-1-carboxylate

To a solution of 4-((5-(2-(2,6-dioxo-1-((2-(trimethylsilyl)ethoxy)methyl)piperidin-3-yl)-1-oxoisoindolin-4-yl)pent-4-yn-1-yl)oxy)-2-fluorobenzamide (Intermediate 880, 0.5 g, 0.84 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.38 g, 1.01 mmol) followed by cesium carbonate (0.82 g, 2.52 mmol). The reaction mixture was purged with argon for 15 min then Pd₂(dba)₃ (0.11 g, 0.12 mmol) and Xantphos (0.14 g, 0.25 mmol) were added. The reaction mixture was purged with argon for another 10 min and stirred at 100° C. for 2 h. The reaction mixture was then filtered through a celite bed, the filter pad was washed with ethyl acetate (100 mL) and the filtrate concentrated in vacuo. The crude material was purified by combi-flash chromatography, by eluting with 5% MeOH in DCM, to afford tert-butyl 6-(4-((5-(2-(2,6-dioxo-1-((2-(trimethylsilyl)ethoxy)methyl)piperidin-3-yl)-1-oxoisoindolin-4-yl)pent-4-yn-1-yl)oxy)-2-fluorobenzamido)-2-((R)-1-methylpyrrolidin-2-yl)-1H-pyrrolo[3,2-c]pyridine-1-carboxylate (Intermediate 881, 0.6 g, 80%) as a yellow solid. ¹H NMR (400 MHz, DMSO-d₆) δ ppm −0.07 (s, 9H), 0.71-0.83 (m, 2H), 1.53-1.76 (m, 13H), 1.99-2.07 (m, 3H), 2.26-2.38 (m, 5H), 2.62-2.80 (m, 3H), 2.99-3.14 (m, 2H), 3.46-3.51 (m, 2H), 3.88-3.91 (m, 1H), 4.15-4.28 (m, 3H), 4.41-4.48 (m, 1H), 4.97-5.07 (m, 2H), 5.19-5.26 (m, 1H), 6.71 (s, 1H), 6.87-6.98 (m, 2H), 7.49-7.55 (m, 1H), 7.62-7.73 (m, 3H), 8.52 (s, 1H), 8.90 (s, 1H), 10.23 (s, 1H). Mass spec m/z 893.5[M+H]⁺.

### Step 5

### Example 213: 4-((5-(2-(2,6-dioxopiperidin-3-yl)-1-oxoisoindolin-4-yl)pent-4-yn-1-yl)oxy)-2-fluoro-N-(2-((R)-1-methylpyrrolidin-2-yl)-1H-pyrrolo[3,2-c]pyridin-6-yl)benzamide

To a solution of tert-butyl 6-(4-((5-(2-(2,6-dioxo-1-((2-(trimethylsilyl)ethoxy)methyl)piperidin-3-yl)-1-oxoisoindolin-4-yl)pent-4-yn-1-yl)oxy)-2-fluorobenzamido)-2-((R)-1-methylpyrrolidin-2-yl)-1H-pyrrolo[3,2-c]pyridine-1-carboxylate (Intermediate 881, 0.65 g, 0.72 mmol) in acetonitrile (20 mL) was added methanesulfonic acid (0.48 mL, 7.2 mmol) and the reaction mixture was stirred at 60° C. for 2 h. After cooling to room temperature, N,N′-dimethylethylenediamine (0.41 mL, 3.63 mmol) and triethylamine (2.04 mL, 14.56 mmol) were added and the reaction mixture was stirred at room temperature for 3 h. The reaction mixture was concentrated in vacuo and the resultant residue was diluted with ice cold water (50 mL). The resultant precipitate was collected by filtration and dried in vacuo. The crude material was purified by preparative HPLC (Method A) to afford 4-((5-(2-(2,6-dioxopiperidin-3-yl)-1-oxoisoindolin-4-yl)pent-4-yn-1-yl)oxy)-2-fluoro-N-(2-((R)-1-methylpyrrolidin-2-yl)-1H-pyrrolo[3,2-c]pyridin-6-yl)benzamide (Example 213, 0.06 g, 13%) as an off-white solid. ¹H NMR (400 MHz, DMSO-d₆) δ ppm 1.74-1.95 (m, 3H), 1.96-2.09 (m, 3H), 2.11-2.19 (m, 4H), 2.22-2.29 (m, 1H), 2.36-2.45 (m, 2H), 2.53-2.62 (m, 1H), 2.65-2.71 (m, 2H), 2.86-2.94 (m, 1H), 3.13-3.17 (m, 1H), 4.20-4.24 (m, 2H), 4.29-4.34 (m, 1H), 4.42-4.48 (m, 1H), 5.10-5.15 (m, 1H), 6.39 (s, 1H), 6.93 (dd, J=8.40, 2.0 Hz, 1H), 7.00 (dd, J=12.80, 2.0 Hz, 1H), 7.53 (t, J=7.60 Hz, 1H), 7.65-7.77 (m, 3H), 8.19 (s, 1H), 8.46 (s, 1H), 9.96 (s, 1H), 11.00 (s, 1H), 11.38 (s, 1H). Mass spec m/z 663.2 [M+H]⁺.

### Example 214 was Prepared Following Scheme 207

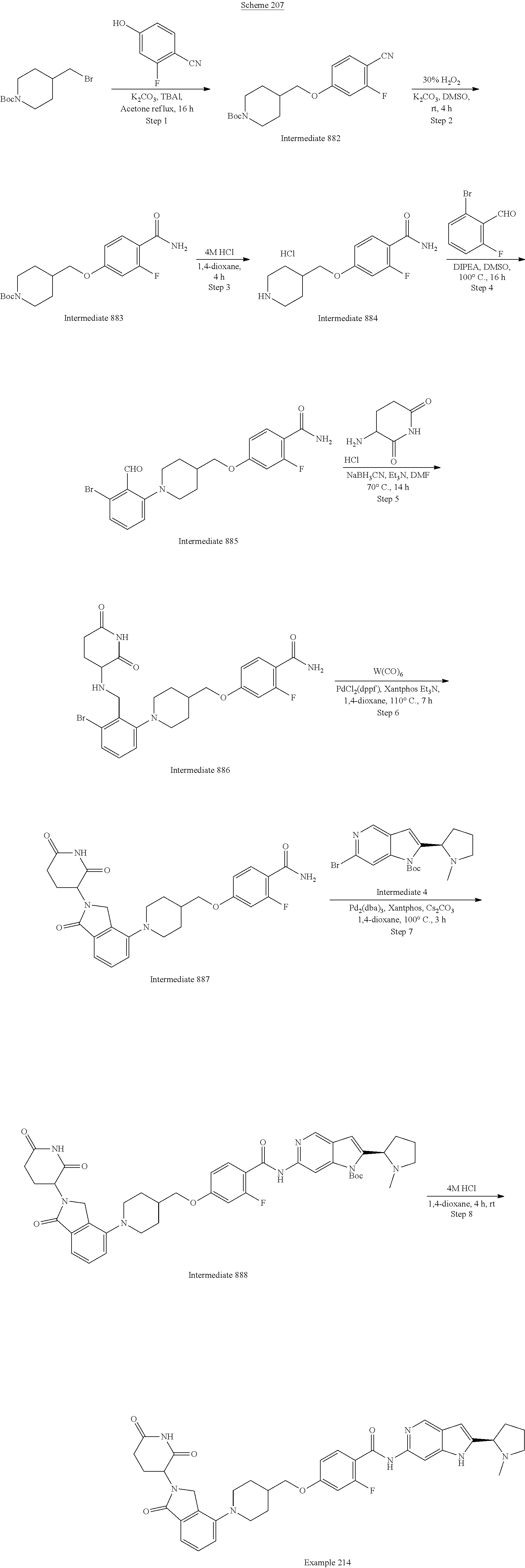

### Step 1

### Intermediate 882: tert-butyl 4-((4-cyano-3-fluorophenoxy)methyl)piperidine-1-carboxylate

To a solution of 2-fluoro-4-hydroxybenzonitrile (CAS No. 82380-18-5, 6.21 g, 45.3 mmol) in acetone (200 mL) was added tert-butyl 4-(bromomethyl)piperidine-1-carboxylate (CAS No. 158407-04-6, 21.0 g, 75.5 mmol), potassium carbonate (41.7 g, 302 mmol) and tetrabutylammonium iodide (2.84 g 7.54 mmol). The reaction mixture was heated at 50° C. for 16 h then concentrated in vacuo. The crude material was purified by combi-flash chromatography, by eluting with 30% ethyl acetate in heptane, to afford tert-butyl 4-((4-cyano-3-fluorophenoxy)methyl)piperidine-1-carboxylate (Intermediate 882, 18.0 g, 71%) as an off-white solid. ¹H NMR (400 MHz, DMSO-d₆) δ ppm 1.08-1.12 (m, 2H), 1.39 (s, 9H), 1.69-1.74 (m, 2H), 1.90-1.94 (m, 1H), 2.70-2.75 (m, 2H), 3.94-3.98 (m, 4H), 6.96 (d, J=7.2 Hz, 1H), 7.15 (d, J=8.40 Hz, 1H), 7.81 (t, J=8.40 Hz, 1H). Mass spec m/z 279.0 [M−56+H]⁺.

### Step 2

### Intermediate 883: tert-butyl 4-((4-carbamoyl-3-fluorophenoxy)methyl)piperidine-1-carboxylate

To a cooled (0° C.) solution of tert-butyl 4-((4-cyano-3-fluorophenoxy)methyl)piperidine-1-carboxylate (Intermediate 882, 12.5 g, 37.4 mmol) in dimethyl sulfoxide (100 mL) was added potassium carbonate (10.3 g, 74.8 mmol) followed by 30% hydrogen peroxide (3.44 mL, 112 mmol) and the reaction mixture was stirred at room temperature for 4 h. The reaction mixture was quenched with ice cold water (300 mL), the resultant precipitate was collected by filtration and dried in vacuo to afford tert-butyl 4-((4-carbamoyl-3-fluorophenoxy)methyl)piperidine-1-carboxylate (Intermediate 883, 14.0 g) as a white solid, which was used for the next step without further purification. ¹H NMR (400 MHz, DMSO-d₆) δ ppm 1.08-1.19 (m, 2H), 1.39 (s, 9H), 1.70-1.76 (m, 2H), 1.85-1.95 (m, 1H), 2.64-2.80 (m, 2H), 3.86-4.00 (m, 4H), 6.82-6.89 (m, 2H), 7.40-7.46 (m, 2H), 7.66 (t, J=8.40 Hz, 1H). Mass spec m/z 297.2 [M−56+H]⁺.

### Step 3

### Intermediate 884: 2-fluoro-4-(piperidin-4-ylmethoxy)benzamide hydrochloride

To a cooled (0° C.) solution of tert-butyl 4-((4-carbamoyl-3-fluorophenoxy)methyl)piperidine-1-carboxylate (Intermediate 883, 14.0 g, 39.7 mmol) in 1,4-dioxane (140 mL) was added 4M hydrochloric acid in 1,4-dioxane (140 mL) and the reaction mixture was stirred at room temperature for 4 h. The reaction mixture was then concentrated in vacuo to afford 2-fluoro-4-(piperidin-4-ylmethoxy)benzamide hydrochloride (Intermediate 884 9.90 g) as an off-white solid, which was used for the next step without further purification. Mass spec m/z 253.2 [M+H]⁺.

### Step 4

### Intermediate 885: 4-((1-(3-bromo-2-formylphenyl)piperidin-4-yl)methoxy)-2-fluorobenzamide

To a solution of 2-fluoro-4-(piperidin-4-ylmethoxy)benzamide hydrochloride (Intermediate 884, 5.0 g, 20.0 mmol) in dimethyl sulfoxide (40 mL) was added N,N-diisopropylethylamine (10 mL, 70.0 mmol) followed by 2-bromo-6-fluorobenzaldehyde (CAS No. 360575-28-6, 4.0 g, 20 mmol) and the reaction mixture was heated at 100° C. for 16 h. The reaction mixture was quenched with water (250 mL), the resultant precipitate was collected by filtration and dried in vacuo. The crude material was purified by combi-flash chromatography, by eluting with 100% ethyl acetate, to afford 4-((1-(3-bromo-2-formylphenyl)piperidin-4-yl)methoxy)-2-fluorobenzamide (Intermediate 885, 2.50 g, 30%) as a yellow solid. ¹H NMR (400 MHz, DMSO-d₆) δ ppm 1.42-1.56 (m, 2H), 1.83-1.95 (m, 3H), 2.86-2.93 (m, 2H), 3.15-3.24 (m, 2H), 3.96-3.99 (m, 2H), 6.84-6.93 (m, 2H), 7.24 (d, J=8.00 Hz, 1H), 7.32 (d, J=8.00 Hz, 1H), 7.41-7.48 (m, 3H), 7.67 (t, J=8.80 Hz, 1H), 10.06 (s, 1H). Mass spec m/z 435.1 [M+H]⁺.

### Step 5

### Intermediate 886: 4-((1-(3-bromo-2-(((2,6-dioxopiperidin-3-yl)amino)methyl)phenyl)piperidin-4-yl)methoxy)-2-fluorobenzamide

To a solution of 4-((1-(3-bromo-2-formylphenyl)piperidin-4-yl)methoxy)-2-fluorobenzamide (Intermediate 885, 2.5 g, 5.70 mmol) in dimethylformamide (30 mL) was added triethylamine (3.2 mL, 23.0 mmol) followed by 3-aminopiperidine-2,6-dione hydrochloride (CAS No. 24666-56-6, 0.95 g, 5.70 mmol). The reaction mixture was heated at 70° C. for 2 h. Sodium cyanoborohydride (1.1 g, 17.1 mmol) was then added and reaction mixture was heated at 70° C. for 12 h. The reaction mixture was diluted with water (100 mL) and extracted with ethyl acetate (3×100 mL). The combined organic layers were dried over anhydrous Na₂SO₄, filtered, and concentrated in vacuo. The crude material was purified by combi-flash chromatography, by eluting with 100% ethyl acetate, to afford 4-((1-(3-bromo-2-(((2,6-dioxopiperidin-3-yl)amino)methyl)phenyl)piperidin-4-yl)methoxy)-2-fluorobenzamide (Intermediate 886, 1.9 g, 60%) as a yellow solid. ¹H NMR (400 MHz, DMSO-d₆) δ ppm 1.39-1.56 (m, 4H), 1.68-1.93 (m, 5H), 2.29-2.32 (m, 1H), 2.52-2.57 (m, 2H), 2.78-2.85 (m, 2H), 2.91-2.96 (m, 1H), 3.12-3.16 (m, 1H), 3.24-3.27 (m, 1H), 3.88-3.89 (m, 2H), 6.85-6.92 (m, 2H), 7.14-7.20 (m, 2H), 7.33-7.36 (m, 1H), 7.40-7.47 (m, 2H), 7.65-7.68 (m, 1H), 10.78 (s, 1H). Mass spec m/z 547.2 [M+H]⁺.

### Step 6

### Intermediate 887: 4-((1-(2-(2,6-dioxopiperidin-3-yl)-1-oxoisoindolin-4-yl)piperidin-4-yl)methoxy)-2-fluorobenzamide

To a solution of 4-((1-(3-bromo-2-(((2,6-dioxopiperidin-3-yl)amino)methyl)phenyl)piperidin-4-yl)methoxy)-2-fluorobenzamide (Intermediate 886, 2.0 g, 3.65 mmol) in 1,4-dioxane (30 mL) was added triethylamine (1.54 mL, 10.96 mmol). The reaction mixture was purged with argon for 15 min then Xantphos (0.65 g, 1.09 mmol) followed by PdCl₂(dppf) (0.56 g, 0.73 mmol) and tungsten hexacarbonyl (0.53 g, 1.46 mmol) were added. The reaction mixture was further purged with argon for 10 min and heated at 110° C. for 16 h. The reaction mixture was then filtered through a celite bed and the filter pad was washed with ethyl acetate (200 mL). The filtrate was concentrated in vacuo and the crude material purified by combi-flash chromatography, by eluting with 100% ethyl acetate, to afford 4-((1-(2-(2,6-dioxopiperidin-3-yl)-1-oxoisoindolin-4-yl)piperidin-4-yl)methoxy)-2-fluorobenzamide (Intermediate 887, 1.5 g, 83%) as a brown solid. ¹H NMR (400 MHz, DMSO-d₆) δ ppm 1.42-1.51 (m, 2H), 1.68-2.01 (m, 4H), 2.40-2.53 (m, 3H), 2.66-2.94 (m, 3H), 3.39-3.49 (m, 1H), 3.96-4.00 (m, 2H), 4.20-4.55 (m, 2H), 5.09-5.13 (m, 1H), 6.85-6.97 (m, 2H), 7.17-7.20 (m, 1H), 7.30-7.33 (m, 1H), 7.40-7.68 (m, 4H), 10.98 (s, 1H). Mass spec m/z 495.1 [M+H]⁺.

### Step 7

### Intermediate 888: tert-butyl 6-(4-((1-(2-(2,6-dioxopiperidin-3-yl)-1-oxoisoindolin-4-yl)piperidin-4-yl)methoxy)-2-fluorobenzamido)-2-((R)-1-methylpyrrolidin-2-yl)-1H-pyrrolo[3,2-c]pyridine-1-carboxylate

To a solution of 4-((1-(2-(2,6-dioxopiperidin-3-yl)-1-oxoisoindolin-4-yl)piperidin-4-yl)methoxy)-2-fluorobenzamide (Intermediate 887, 0.41 g, 0.84 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.4 g, 1.05 mmol) followed by cesium carbonate (1.03 g, 3.15 mmol). The reaction mixture was purged with argon for 15 min then Pd₂(dba)₃ (0.14 g, 0.15 mmol) followed by Xantphos (0.18 g, 0.31 mmol) were added. The reaction mixture was further purged with argon for 10 min and heated at 100° C. for 3 h. The reaction mixture was then filtered through a celite bed, the filter pad was washed with ethyl acetate (100 mL) and the filtrate concentrated in vacuo. The crude material was purified by combi-flash chromatography, by eluting with 100% ethyl acetate, to afford tert-butyl 6-(4-((1-(2-(2,6-dioxopiperidin-3-yl)-1-oxoisoindolin-4-yl)piperidin-4-yl)methoxy)-2-fluorobenzamido)-2-((R)-1-methylpyrrolidin-2-yl)-1H-pyrrolo[3,2-c]pyridine-1-carboxylate (Intermediate 888, 0.3 g, 36%) as an off-white solid. Mass spec m/z 794.5 [M+H]⁺.

### Step 8

### Example 214: 4-((1-(2-(2,6-dioxopiperidin-3-yl)-1-oxoisoindolin-4-yl)piperidin-4-yl)methoxy)-2-fluoro-N-(2-((R)-1-methylpyrrolidin-2-yl)-1H-pyrrolo[3,2-c]pyridin-6-yl)benzamide

To a solution of tert-butyl 6-(4-((1-(2-(2,6-dioxopiperidin-3-yl)-1-oxoisoindolin-4-yl)piperidin-4-yl)methoxy)-2-fluorobenzamido)-2-((R)-1-methylpyrrolidin-2-yl)-1H-pyrrolo[3,2-c]pyridine-1-carboxylate (Intermediate 888, 0.25 g, 0.31 mmol) in 1,4 dioxane (5 mL) was added 4M hydrochloric acid in 1,4-dioxane (5 mL). The reaction mixture was stirred at room temperature for 4 h then concentrated in vacuo. The crude material was purified by preparative HPLC (Method A) to afford 4-((1-(2-(2,6-dioxopiperidin-3-yl)-1-oxoisoindolin-4-yl)piperidin-4-yl)methoxy)-2-fluoro-N-(2-((R)-1-methylpyrrolidin-2-yl)-1H-pyrrolo[3,2-c]pyridin-6-yl)benzamide (Example 214, 0.04 g, 18%) as an off-white solid. ¹H NMR (400 MHz, DMSO-d) δ ppm 1.43-1.53 (m, 2H), 1.74-2.02 (m, 6H), 2.12-2.18 (m, 4H), 2.21-2.28 (m, 1H), 2.42-2.48 (m, 1H), 2.52-2.61 (m, 2H), 2.74-2.81 (m, 2H), 2.87-2.95 (m, 1H), 3.13-3.17 (m, 1H), 3.27-3.29 (m, 1H), 3.39-3.49 (m, 2H), 4.01-4.03 (m, 2H), 4.29-4.33 (m, 1H), 4.43-4.48 (m, 1H), 5.09-5.14 (m, 1H), 6.38 (s, 1H), 6.91-7.00 (m, 2H), 7.19 (d, J=7.60 Hz, 1H), 7.31 (d, J=7.20 Hz, 1H), 7.44 (t, J=7.6 Hz, 1H), 7.76 (d, J=8.80 Hz, 1H), 8.19 (s, 1H), 8.46 (s, 1H), 9.96 (d, J=5.6 Hz, 1H), 10.99 (s, 1H), 11.27 (s, 1H). Mass spec m/z 694.1 [M+H]⁺.

### Example 215 was Prepared Following Scheme 207

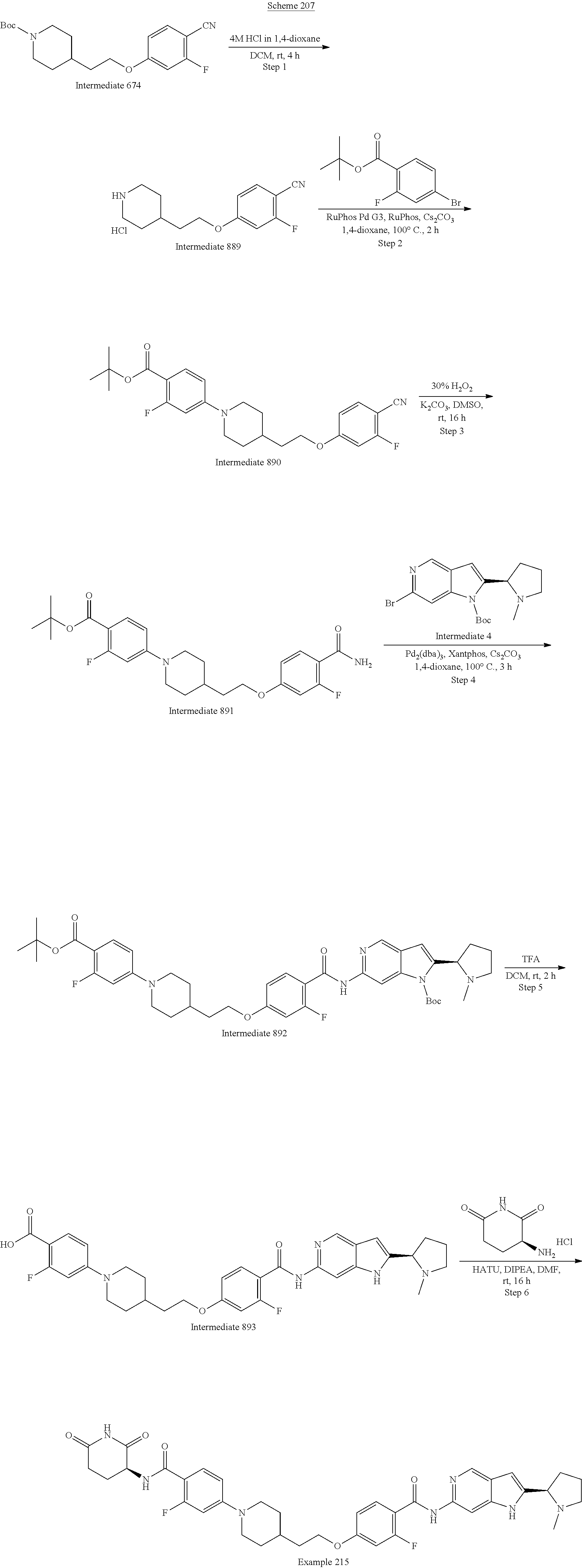

### Step 1

### Intermediate 889: 2-fluoro-4-(2-(piperidin-4-yl)ethoxy)benzonitrile hydrochloride

To a solution of tert-butyl 4-(2-(4-cyano-3-fluorophenoxy)ethyl)piperidine-1-carboxylate (Intermediate 674, 9.0 g, 25.8 mmol) in dichloromethane (40 mL) was added 4M hydrochloride in 1,4-dioxane (20 mL) and the reaction mixture was stirred at room temperature for 2 h. The reaction mixture was concentrated in vacuo to afford 2-fluoro-4-(2-(piperidin-4-yl)ethoxy)benzonitrile hydrochloride (Intermediate 889, 6.0 g, 81%) as an off-white solid, which was used for the next step without further purification. Mass spec m/z 249.0 [M+H]⁺.

### Step 2

### Intermediate 890: tert-butyl 4-(4-(2-(4-cyano-3-fluorophenoxy)ethyl)piperidin-1-yl)-2-fluorobenzoate

To a solution of 2-fluoro-4-(2-(piperidin-4-yl)ethoxy)benzonitrile hydrochloride (Intermediate 889, 3.0 g, 11 mmol) in 1,4-dioxane (30 mL) was added tert-butyl 4-bromo-2-fluorobenzoate (CAS No. 889858-12-2, 3.5 g, 13 mmol) followed by cesium carbonate (6.1 g, 32 mmol). The reaction mixture was purged with argon for 15 min then RuPhos (0.75 g, 1.6 mmol) and RuPhos Pd G3 (0.93 g, 1.1 mmol) were added. The reaction mixture was purged with argon for another 10 min and stirred at 100° C. for 2 h. The reaction mixture was concentrated in vacuo. The crude material was purified by combi-flash chromatography, by eluting with 5% MeOH in DCM, to afford tert-butyl 4-(4-(2-(4-cyano-3-fluorophenoxy)ethyl)piperidin-1-yl)-2-fluorobenzoate (Intermediate 890, 3.5 g, 36%) as a brown solid. ¹H NMR (400 MHz, DMSO-d₆) δ ppm 1.19-1.25 (m, 2H), 1.49 (s, 9H), 1.68-1.78 (m, 5H), 2.81-2.86 (m, 2H), 3.88-3.91 (m, 2H), 4.14-4.17 (m, 2H), 6.66-6.76 (m, 2H), 6.96-6.99 (m, 1H), 7.16-7.19 (m, 1H), 7.58-7.62 (m, 1H), 7.79-7.83 (m, 1H). Mass spec m/z 443.0 [M+H]⁺.

### Step 3

### Intermediate 891: tert-butyl 4-(4-(2-(4-carbamoyl-3-fluorophenoxy)ethyl)piperidin-1-yl)-2-fluorobenzoate

To a solution of tert-butyl 4-(4-(2-(4-cyano-3-fluorophenoxy)ethyl)piperidin-1-yl)-2-fluorobenzoate (Intermediate 890, 2.5 g, 5.65 mmol) in dimethyl sulfoxide (10 mL) was added potassium carbonate (1.94 g, 14.1 mmol) followed by 30% hydrogen peroxide (4.42 mL, 56.6 mmol) and the reaction mixture was stirred at room temperature for 16 h. The reaction mixture was poured into ice cold water (100 mL). The resultant precipitate was collected by filtration and dried in vacuo to afford tert-butyl 4-(4-(2-(4-carbamoyl-3-fluorophenoxy)ethyl)piperidin-1-yl)-2-fluorobenzoate (Intermediate 891, 2.2 g, 85%) as an off-white solid, which was used for the next step without further purification. ¹H NMR (400 MHz, DMSO-d₆) δ ppm 1.16-1.23 (m, 2H), 1.49 (s, 9H), 1.67-1.78 (m, 5H), 2.80-2.87 (m, 2H), 3.87-3.92 (m, 2H), 4.07-4.10 (m, 2H), 6.65-6.75 (m, 2H), 6.82-6.89 (m, 2H), 7.40 (br s, 1H), 7.46 (br s, 1H), 7.58-7.69 (m, 2H). Mass spec m/z 461.1 [M+H]⁺.

### Step 4

### Intermediate 892: tert-butyl (R)-6-(4-(2-(1-(4-(tert-butoxycarbonyl)-3-fluorophenyl)piperidin-4-yl)ethoxy)-2-fluorobenzamido)-2-(1-methylpyrrolidin-2-yl)-1H-pyrrolo[3,2-c]pyridine-1-carboxylate

To a solution of tert-butyl 4-(4-(2-(4-carbamoyl-3-fluorophenoxy)ethyl)piperidin-1-yl)-2-fluorobenzoate (Intermediate 891, 1.80 g, 3.60 mmol) 1,4-dioxane (20 mL) was added tert-butyl (R)-6-bromo-2-(1-methylpyrrolidin-2-yl)-1H-pyrrolo[3,2-c]pyridine-1-carboxylate (Intermediate 4, 1.80 g, 4.50 mmol) followed by cesium carbonate (2.70 g, 9.00 mmol). The reaction mixture was purged with argon for 15 min then XantPhos (0.72 g, 0.90 mmol) and Pd₂(dba)₃ (0.54 g, 0.63 mmol) were added. The reaction mixture was purged with argon for another 10 min and stirred at 100° C. for 3 h. The reaction mixture was concentrated in vacuo and the crude material was purified by combi-flash chromatography, by eluting with 5% MeOH in DCM, to afford tert-butyl (R)-6-(4-(2-(1-(4-(tert-butoxycarbonyl)-3-fluorophenyl)piperidin-4-yl)ethoxy)-2-fluorobenzamido)-2-(1-methylpyrrolidin-2-yl)-1H-pyrrolo[3,2-c]pyridine-1-carboxylate (Intermediate 892, 1.69 g, 62%) as an off-white solid. Mass spec m/z 760.6 [M+H]⁺.

### Step 5

### Intermediate 893: (R)-2-fluoro-4-(4-(2-(3-fluoro-4-((2-(1-methylpyrrolidin-2-yl)-1H-pyrrolo[3,2-c]pyridin-6-yl)carbamoyl)phenoxy)ethyl)piperidin-1-yl)benzoic acid

To a cooled (0° C.) solution of tert-butyl (R)-6-(4-(2-(1-(4-(tert-butoxycarbonyl)-3-fluorophenyl)piperidin-4-yl)ethoxy)-2-fluorobenzamido)-2-(1-methylpyrrolidin-2-yl)-1H-pyrrolo[3,2-c]pyridine-1-carboxylate (Intermediate 892, 0.6 g, 0.8 mmol) in dichloromethane (3 mL) was added trifluoroacetic acid (4.0 mL) and the reaction mixture was stirred at room temperature for 2 h. The reaction mixture was then concentrated in vacuo. The crude material was triturated with diethyl ether (5×2 mL) and dried in vacuo to afford as (R)-2-fluoro-4-(4-(2-(3-fluoro-4-((2-(1-methylpyrrolidin-2-yl)-1H-pyrrolo[3,2-c]pyridin-6-yl)carbamoyl)phenoxy)ethyl)piperidin-1-yl)benzoic acid (Intermediate 893, 0.4 g, 80%) as a brown semi-solid. ¹H NMR (400 MHz, DMSO-d₆) δ ppm 1.20-1.28 (m, 3H), 1.61-1.81 (m, 5H), 2.15-2.20 (m, 2H), 2.30-2.35 (m, 1H), 2.81-2.85 (m, 2H), 2.87 (s, 3H), 3.23-3.34 (m, 1H), 3.51-3.55 (m, 1H), 3.91-3.95 (m, 2H), 4.15-4.19 (m, 2H), 4.75-4.85 (m, 1H), 6.72-6.78 (m, 3H), 6.98-7.31 (m, 6H), 9.04 (br s, 1H), 11.48 (br s, 1H), 12.95 (br s, 1H). Mass spec m/z 604.3 [M+H]⁺.

### Step 6

### Example 215: N—((S)-2,6-dioxopiperidin-3-yl)-2-fluoro-4-(4-(2-(3-fluoro-4-((2-((R)-1-methylpyrrolidin-2-yl)-1H-pyrrolo[3,2-c]pyridin-6-yl)carbamoyl)phenoxy)ethyl)piperidin-1-yl)benzamide

To a cooled (0° C.) solution of (R)-2-fluoro-4-(4-(2-(3-fluoro-4-((2-(1-methylpyrrolidin-2-yl)-1H-pyrrolo[3,2-c]pyridin-6-yl)carbamoyl)phenoxy)ethyl)piperidin-1-yl)benzoic acid-2,2,2-trifluoroacetic acid (Intermediate 893, 0.5 g, 0.8 mmol) in dimethylformamide (5 mL) was added HATU (0.3 g, 0.9 mmol) and N,N-diisopropylethylamine (0.7 mL, 4 mmol) and the reaction mixture was stirred at 0° C. for 10 min. (S)-3-Aminopiperidine-2,6-dione hydrochloride (CAS No. 25181-50-4, 0.1 g, 1.0 mmol) was then added and the reaction mixture was stirred at room temperature for 16 h. The reaction mixture was then poured into ice cold water (50 mL). The resultant precipitate was collected by filtration and dried in vacuo. The crude material was purified by preparative HPLC (Method A) to afford N—((S)-2,6-dioxopiperidin-3-yl)-2-fluoro-4-(4-(2-(3-fluoro-4-((2-((R)-1-methylpyrrolidin-2-yl)-1H-pyrrolo[3,2-c]pyridin-6-yl)carbamoyl)phenoxy)ethyl)piperidin-1-yl)benzamide (Example 215, 0.277 g, 50%) as a white solid. ¹H NMR (400 MHz, DMSO-d₆) δ ppm 1.24-1.29 (m, 2H), 1.71-1.93 (m, 8H), 1.98-2.02 (m, 1H), 2.03-2.12 (m, 2H), 2.15 (s, 3H), 2.31-2.39 (m, 1H), 2.53-2.57 (m, 1H), 2.72-2.85 (m, 3H), 3.11-3.15 (m, 1H), 3.28-3.30 (m, 1H), 3.88-3.92 (m, 2H), 4.12-4.15 (m, 2H), 4.69-4.75 (m, 1H), 6.39 (s, 1H), 6.74-6.78 (m, 1H), 6.80-6.83 (m, 1H), 6.89-6.92 (m, 1H), 6.94-6.98 (m, 1H), 7.59-7.64 (m, 1H), 7.73-7.77 (m, 1H), 7.97-8.01 (m, 1H), 8.19 (s, 1H), 8.46 (s, 1H), 9.94 (br s, 1H), 10.83 (br s, 1H), 11.37 (br s, 1H). Mass spec m/z 714.3 [M+H]⁺.

### Example 216 was Prepared Following Scheme 208

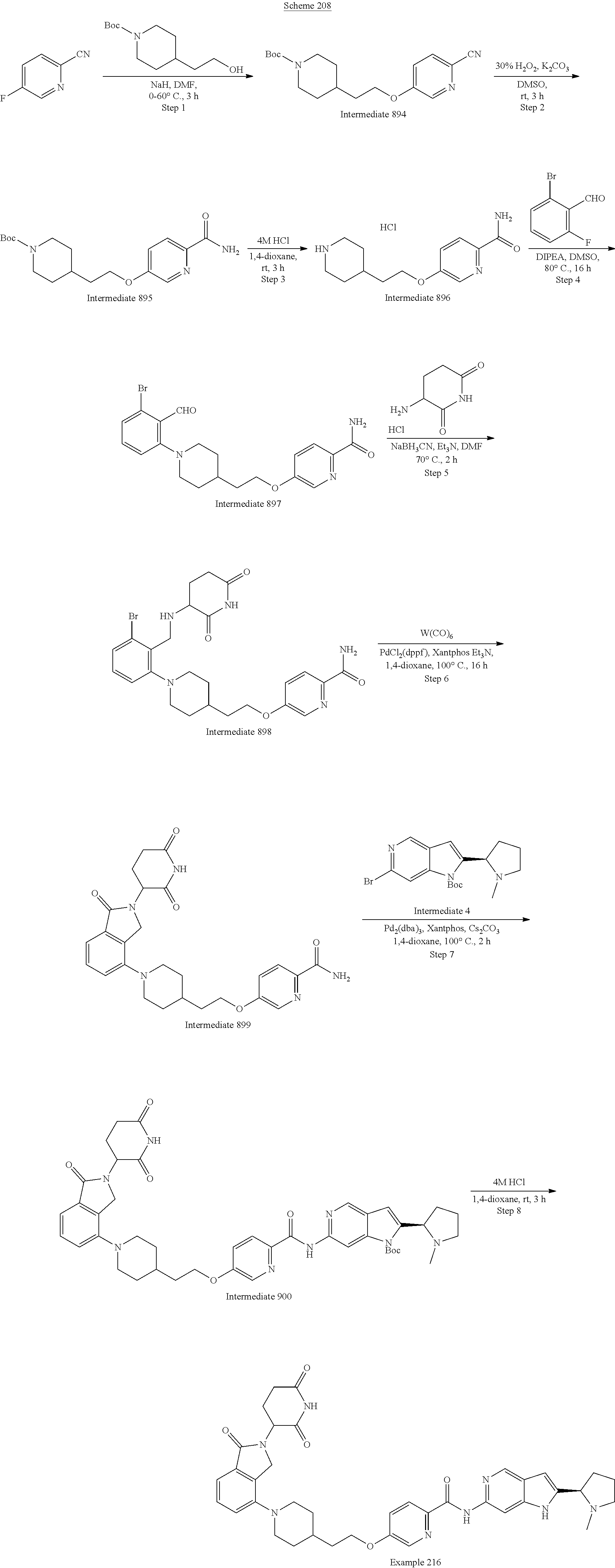

### Step 1

### Intermediate 894: tert-butyl 4-(2-((6-cyanopyridin-3-yl)oxy)ethyl)piperidine-1-carboxylate

To a cooled (0° C.) solution of 5-fluoropicolinonitrile (CAS No. 327056-62-2, 6.38 g, 52.3 mmol) in dimethylformamide (30 mL) was added sodium hydride (60% in mineral oil, 1.74 g, 65.4 mmol) and the reaction mixture was stirred at 0° C. for 30 min. tert-Butyl 4-(2-hydroxyethyl)piperidine-1-carboxylate (CAS No. 89151-44-0, 10.0 g, 43.6 mmol) was then added and the reaction mixture was stirred at 60° C. for 3 h. The reaction mixture was quenched with water (250 mL) and extracted with ethyl acetate (2×125 mL). The combined organic layers were dried over Na₂SO₄, filtered and concentrated in vacuo. The crude material was purified by combi-flash chromatography, by eluting with 50% ethyl acetate in heptane, to afford tert-butyl 4-(2-((6-cyanopyridin-3-yl)oxy)ethyl)piperidine-1-carboxylate (Intermediate 894, 14 g, 97%) as an off-white solid. ¹H NMR (400 MHz, DMSO-d₆) δ ppm 0.98-1.11 (m, 2H), 1.39 (s, 9H), 1.58-1.74 (m, 5H), 2.61-2.77 (m, 2H), 3.87-3.92 (m, 2H), 4.18-4.22 (m, 2H), 7.59 (dd, J=8.80, 2.93 Hz, 1H), 7.99 (d, J=8.80 Hz, 1H), 8.43 (s, 1H).

### Step 2

### Intermediate 895: tert-butyl 4-(2-((6-carbamoylpyridin-3-yl)oxy)ethyl)piperidine-1-carboxylate

To a cooled (0° C.) solution of tert-butyl 4-(2-((6-cyanopyridin-3-yl)oxy)ethyl)piperidine-1-carboxylate (Intermediate 894, 14 g, 42.2 mmol) in dimethyl sulfoxide (50 mL) was added potassium carbonate (17.5 g, 127 mmol) followed by 30% hydrogen peroxide (12.9 mL, 422 mmol) and the reaction mixture was stirred at room temperature for 3 h. The reaction mixture was quenched with ice cold water (300 mL). The resultant precipitate was collected by filtration and dried in vacuo to afford tert-butyl 4-(2-((6-carbamoylpyridin-3-yl)oxy)ethyl)piperidine-1-carboxylate (Intermediate 895, 12 g, 81%) as an off-white solid, which was used for the next step without further purification. ¹H NMR (400 MHz, DMSO-d₆) δ ppm 1.03-1.08 (m, 2H), 1.38 (s, 9H), 1.67-1.72 (m, 5H), 2.67-2.74 (m, 2H), 3.88-3.94 (m, 2H), 4.11-4.16 (m, 2H), 7.46 (br s, 1H), 7.50-7.54 (m, 1H), 7.91 (br s, 1H), 7.97 (d, J=7.83 Hz, 1H), 8.27 (s, 1H). Mass spec m/z 348.1 [M−H]⁻.

### Step 3

### Intermediate 896: 5-(2-(piperidin-4-yl)ethoxy)picolinamide hydrochloride

To a solution of tert-butyl 4-(2-((6-carbamoylpyridin-3-yl)oxy)ethyl)piperidine-1-carboxylate (Intermediate 895, 12 g, 34.3 mmol) in dichloromethane (40 mL) was added 4M hydrochloric acid in 1,4-dioxane (70 mL) and the reaction mixture was stirred at room temperature for 3 h. The reaction mixture was then concentrated in vacuo to afford 5-(2-(piperidin-4-yl)ethoxy)picolinamide hydrochloride (Intermediate 896, 8.0 g, 82%) as an off-white solid, which was used for the next step without further purification. ¹H NMR (401 MHz, DMSO-d₆) δ ppm 1.35-1.44 (m, 2H), 1.65-1.70 (m, 3H), 1.80-1.85 (m, 2H), 2.76-2.82 (m, 2H), 3.17-3.22 (m, 2H), 4.11-4.20 (m, 2H), 7.51-7.55 (m, 2H), 7.95 (br s, 1H), 7.97-8.00 (m, 1H), 8.26 (br s, 1H), 8.94 (br, 1H), 9.14 (br, 1H).

### Step 4

### Intermediate 897: 5-(2-(1-(3-bromo-2-formylphenyl)piperidin-4-yl)ethoxy)picolinamide

To a solution of 5-(2-(piperidin-4-yl)ethoxy)picolinamide hydrochloride (Intermediate 896, 8.0 g, 27.99 mmol) in dimethyl sulfoxide (50 ml) was added N,N-diisopropylethylamine (19.6 mL, 112 mmol) followed by 2-bromo-6-fluorobenzaldehyde (CAS No. 360575-28-6, 5.68 g, 28.0 mmol) and the reaction mixture was heated at 80° C. for 16 h. The reaction mixture was then quenched with water (250 mL) and extracted with ethyl acetate (2×125 mL). The combined organic layers were dried over Na₂SO₄, filtered and concentrated in vacuo. The crude material was purified by combi-flash chromatography, by eluting with 10% MeOH in DCM, to afford 5-(2-(1-(3-bromo-2-formylphenyl)piperidin-4-yl)ethoxy)picolinamide (Intermediate 897, 7.5 g, 62%) as a pale yellow solid. ¹H NMR (400 MHz, DMSO-d₆) δ ppm 1.40-1.45 (m, 2H), 1.61-1.70 (m, 1H), 1.76-1.83 (m, 4H), 2.82-2.88 (m, 2H), 3.16-3.19 (m, 2H), 4.15-4.22 (m, 2H), 7.22 (d, J=8.32 Hz, 1H), 7.31 (d, J=8.20 Hz, 1H), 7.39-7.45 (m, 2H), 7.52-7.54 (d, J=8.40 Hz, 1H), 7.90 (br s, 1H), 7.98 (d, J=8.54 Hz, 1H), 8.28 (s, 1H), 10.04 (br s, 1H). Mass spec m/z 433.8 [M+H+2]⁺.

### Step 5

### Intermediate 898: 5-(2-(1-(3-bromo-2-(((2,6-dioxopiperidin-3-yl)amino)methyl)phenyl)piperidin-4-yl)ethoxy)picolinamide

To a solution of 5-(2-(1-(3-bromo-2-formylphenyl)piperidin-4-yl)ethoxy)picolinamide (Intermediate 897, 7.0 g, 16.2 mmol) in acetonitrile (30 mL) was added 3-aminopiperidine-2,6-dione hydrochloride (CAS No. 24666-56-6, 3.19 g, 19.4 mmol) and the reaction mixture was stirred at 70° C. for 2 h. Sodium cyanoborohydride (3.05 g, 48.6 mmol) was then added and the reaction mixture was heated at 70° C. for 12 h. The reaction mixture was quenched with water (100 mL) and extracted with ethyl acetate (3×100 mL). The combined organic layers were dried over Na₂SO₄, filtered, and concentrated in vacuo. The crude material was purified by combi-flash chromatography, by eluting with 10% MeOH in DCM, to afford 5-(2-(1-(3-bromo-2-(((2,6-dioxopiperidin-3-yl)amino)methyl)phenyl)piperidin-4-yl)ethoxy)picolinamide (Intermediate 898, 5.3 g, 60%) as an off-white solid. Mass spec m/z 546.1 [M+H+2]⁺.

### Step 6

### Intermediate 899: 5-(2-(1-(2-(2,6-dioxopiperidin-3-yl)-1-oxoisoindolin-4-yl)piperidin-4-yl)ethoxy)picolinamide

To a solution of 5-(2-(1-(3-bromo-2-(((2,6-dioxopiperidin-3-yl)amino)methyl)phenyl)piperidin-4-yl)ethoxy)picolinamide (Intermediate 898, 3.2 g, 5.9 mmol) in 1,4-dioxane (20 mL) was added triethylamine (2.5 mL, 18 mmol). The reaction mixture was purged with argon gas for 15 min then Xantphos (0.91 g, 1.2 mmol) followed by PdCl₂(dppf) (0.70 g, 1.2 mmol) and tungsten hexacarbonyl (1.1 g, 2.9 mmol) were added. The reaction mixture was further purged with argon gas for 10 min and heated at 100° C. for 16 h. The reaction mixture was filtered through a celite bed and the filter pad was washed with ethyl acetate (200 mL). The filtrate was concentrated in vacuo and the crude material purified by combi-flash chromatography, by eluting with 12% MeOH in DCM, to afford 5-(2-(1-(2-(2,6-dioxopiperidin-3-yl)-1-oxoisoindolin-4-yl)piperidin-4-yl)ethoxy)picolinamide (Intermediate 899, 2.0 g, 69%) as a brown solid. ¹H NMR (400 MHz, DMSO-d₆) δ ppm 1.62-1.67 (m, 2H), 1.74-1.84 (m, 5H), 1.97-1.98 (m, 2H), 2.63-2.68 (m, 3H), 2.70-2.76 (m, 3H), 2.86-2.89 (m, 1H), 4.19-4.21 (m, 2H), 4.29-4.32 (m, 1H), 4.43-4.43 (m, 1H), 7.15 (d, J=8.24 Hz, 1H), 7.26 (d, J=8.24 Hz, 1H), 7.39-7.44 (m, 2H), 7.52 (d, J=8.28 Hz, 1H), 7.89 (br s, 1H), 7.96 (d, J=8.40 Hz, 1H), 8.27 (s, 1H), 10.95 (br s, 1H). Mass spec m/z 492.0 [M+H]⁺.

### Step 7

### Intermediate 900: tert-butyl 6-(5-(2-(1-(2-(2,6-dioxopiperidin-3-yl)-1-oxoisoindolin-4-yl)piperidin-4-yl)ethoxy)picolinamido)-2-((R)-1-methylpyrrolidin-2-yl)-1H-pyrrolo[3,2-c]pyridine-1-carboxylate

To a solution of 5-(2-(1-(2-(2,6-dioxopiperidin-3-yl)-1-oxoisoindolin-4-yl)piperidin-4-yl)ethoxy)picolinamide (Intermediate 889, 0.5 g, 0.95 mmol) in 1,4-dioxane (20 mL) was added tert-butyl (R)-6-bromo-2-(1-methylpyrrolidin-2-yl)-1H-pyrrolo[3,2-c]pyridine-1-carboxylate (Intermediate 4, 0.066 g, 0.17 mmol) followed by cesium carbonate (0.93 g, 2.85 mmol). The reaction mixture was purged with argon for 15 min then Pd₂(dba)₃ (0.13 g, 0.14 mmol) and Xantphos (0.16 g, 0.28 mmol) were added. The reaction mixture was purged with argon for another 10 min and stirred at 100° C. for 2 h. The reaction mixture was filtered through a celite bed, the filter cake was washed with ethyl acetate (100 mL) and the filtrate concentrated in vacuo. The crude material was purified by combi-flash chromatography, by eluting with 9% MeOH in DCM, to afford tert-butyl 6-(5-(2-(1-(2-(2,6-dioxopiperidin-3-yl)-1-oxoisoindolin-4-yl)piperidin-4-yl)ethoxy)picolinamido)-2-((R)-1-methylpyrrolidin-2-yl)-1H-pyrrolo[3,2-c]pyridine-1-carboxylate (Intermediate 900, 0.6 g, 76%) as an off-white solid. Mass spec m/z 791.3 [M+H]⁺.

### Step-8

### Example 216: 5-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)picolinamide

To a solution of tert-butyl 6-(5-(2-(1-(2-(2,6-dioxopiperidin-3-yl)-1-oxoisoindolin-4-yl)piperidin-4-yl)ethoxy)picolinamido)-2-((R)-1-methylpyrrolidin-2-yl)-1H-pyrrolo[3,2-c]pyridine-1-carboxylate (Intermediate 900, 0.15 g, 0.18 mmol) in dichloromethane (30 mL) was added 4M hydrochloric acid in 1,4-dioxane (3.0 mL) and the reaction mixture was stirred at room temperature for 3 h. The reaction mixture was concentrated in vacuo. The crude material was purified by preparative HPLC (Method A) to afford 5-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)picolinamide (Example 216, 0.015 g, 12%) as an off-white solid. ¹H NMR (400 MHz, DMSO-d₆) δ ppm 1.35-1.47 (m, 2H), 1.63-1.73 (m, 1H), 1.77-1.83 (m, 3H), 1.83-1.91 (m, 4H), 1.94-2.04 (m, 2H), 2.16 (s, 3H), 2.23-2.29 (m, 1H), 2.59-2.64 (m, 2H), 2.71-2.80 (m, 2H), 2.86-2.97 (m, 2H), 3.11-3.17 (m, 1H), 3.37-3.44 (m, 2H), 4.23-4.34 (m, 3H), 4.40-4.48 (m, 1H), 5.07-5.15 (m, 1H), 6.40 (s, 1H), 7.18 (d, J=8.00 Hz, 1H), 7.30 (d, J=7.63 Hz, 1H), 7.39-7.47 (m, 1H), 7.67 (dd, J=8.63, 2.88 Hz, 1H), 8.17 (d, J=8.50 Hz, 1H), 8.26 (s, 1H), 8.44 (s, 1H), 8.48 (s, 1H), 10.14 (br s, 1H), 10.97 (br s, 1H), 11.43 (br s, 1H). Mass spec m/z 691.3 [M+H]⁺.

### Example 217 was Prepared Following Scheme 209

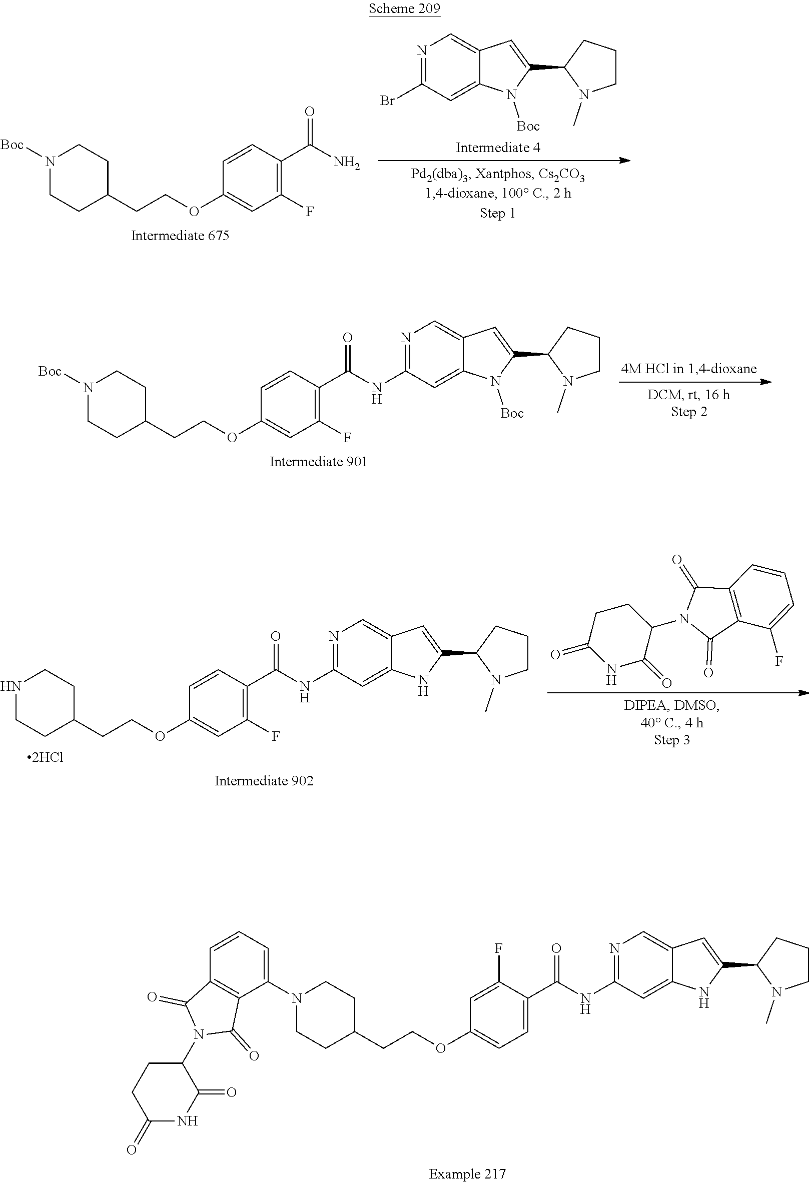

### Step 1

### Intermediate 901: tert-butyl (R)-6-(4-(2-(1-(tert-butoxycarbonyl)piperidin-4-yl)ethoxy)-2-fluorobenzamido)-2-(1-methylpyrrolidin-2-yl)-1H-pyrrolo[3,2-c]pyridine-1-carboxylate

To a solution of tert-butyl 4-(2-(4-carbamoyl-3-fluorophenoxy)ethyl)piperidine-1-carboxylate (Intermediate 675, 0.5 g, 1.0 mmol) in 1,4-dioxane (5 mL) were added tert-butyl (R)-6-bromo-2-(1-methylpyrrolidin-2-yl)-1H-pyrrolo[3,2-c]pyridine-1-carboxylate (Intermediate 4, 0.06 g, 2.0 mmol) followed by cesium carbonate (1.0 g, 4.0 mmol). The reaction mixture was purged with argon for 15 min then Pd₂(dba)₃ (0.2 g, 0.2 mmol) and Xantphos (0.2 g, 0.4 mmol) were added. The reaction mixture was further purged with argon for 10 min and heated at 100° C. for 2 h. The reaction mixture was filtered through a celite bed, the filter pad was washed with ethyl acetate (100 mL) and the filtrate concentrated in vacuo. The crude material was purified by combi-flash chromatography, by eluting with 50% ethyl acetate in heptane, to afford tert-butyl (R)-6-(4-(2-(1-(tert-butoxycarbonyl)piperidin-4-yl)ethoxy)-2-fluorobenzamido)-2-(1-methylpyrrolidin-2-yl)-1H-pyrrolo[3,2-c]pyridine-1-carboxylate (Intermediate 901, 0.75 g, 60%) as an off-white solid. ¹H NMR (400 MHz, DMSO-d₆) δ ppm 0.98-1.10 (m, 2H), 1.34-1.41 (m, 12H), 1.43-1.52 (m, 1H), 1.55-1.79 (m, 14H), 2.38-2.40 (m, 4H), 2.58-2.78 (m, 2H), 3.08-3.14 (m, 2H), 3.85-3.94 (m, 2H), 4.06-4.02 (m, 2H), 6.72 (s, 1H), 6.86 (d, J=9.20 Hz, 1H), 6.93 (d, J=8.80 Hz, 1H), 7.70 (t, J=9.20 Hz, 1H), 8.53 (s, 1H), 8.90 (s, 1H), 10.24 (s, 1H). Mass spec m/z 666.3 [M+H]⁺.

### Step 3

### Intermediate 902: (R)-2-fluoro-N-(2-(1-methylpyrrolidin-2-yl)-1H-pyrrolo[3,2-c]pyridin-6-yl)-4-(2-(piperidin-4-yl)ethoxy)benzamide hydrochloride

To a cooled (0° C.) solution of tert-butyl (R)-6-(4-(2-(1-(tert-butoxycarbonyl)piperidin-4-yl)ethoxy)-2-fluorobenzamido)-2-(1-methylpyrrolidin-2-yl)-1H-pyrrolo[3,2-c]pyridine-1-carboxylate (Intermediate 901, 1.80 g, 2.70 mmol) in dichloromethane (18 mL) was added 4M hydrochloric acid in dioxane (18 mL) and the reaction mixture was stirred at room temperature for 2 h. The reaction mixture was then concentrated in vacuo and the residue triturated with n-pentane (20 mL) to afford (R)-2-fluoro-N-(2-(1-methylpyrrolidin-2-yl)-1H-pyrrolo[3,2-c]pyridin-6-yl)-4-(2-(piperidin-4-yl)ethoxy)benzamide hydrochloride (Intermediate 902, 2.0 g) as an off-white solid, which was used for the next step without further purification. Mass spec m/z 466.3 [M+H]⁺.

### Step 3

### Example 217: 4-(2-(1-(2-(2,6-dioxopiperidin-3-yl)-1,3-dioxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide

To a solution of (R)-2-fluoro-N-(2-(1-methylpyrrolidin-2-yl)-1H-pyrrolo[3,2-c]pyridin-6-yl)-4-(2-(piperidin-4-yl)ethoxy)benzamide hydrochloride (Intermediate 902, 0.3 g, 0.6 mmol) in dimethyl sulfoxide (3 mL) was added N,N-diisopropylethylamine (0.4 mL, 2.0 mmol) followed by 2-(2,6-dioxopiperidin-3-yl)-4-fluoroisoindoline-1,3-dione (CAS No. 835616-60-9, 0.2 g, 0.6 mmol) and the reaction mixture was heated at 40° C. for 4 h. The reaction mixture was quenched with water (50 mL), the resultant precipitate was collected by filtration and dried in vacuo. The crude material was purified by preparative HPLC (Method A) to afford 4-(2-(1-(2-(2,6-dioxopiperidin-3-yl)-1,3-dioxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide (Example 217, 0.017 g, 4%) as an off-white solid. ¹H NMR (400 MHz, DMSO-d₆) δ ppm 1.40-1.50 (m, 2H), 1.65-1.92 (m, 8H), 2.01-2.07 (m, 1H), 2.12-2.18 (m, 4H), 2.21-2.28 (m, 1H), 2.52-2.61 (m, 4H), 2.83-2.91 (m, 3H), 3.11-3.17 (m, 1H), 3.68-3.72 (m, 2H), 4.15-4.18 (m, 2H), 5.06-5.11 (m, 1H), 6.38 (s, 1H), 6.92 (dd, J=8.80, 2.4 Hz, 1H), 6.97 (dd, J=9.2, 2.0 Hz, 1H), 7.31-7.36 (m, 2H), 7.66-7.70 (m, 1H), 7.55 (t, J=8.80 Hz, 1H), 8.46 (s, 1H), 9.95 (d, J=5.60 Hz, 1H), 11.09 (s, 1H), 11.39 (s, 1H). Mass spec m/z 722.3 [M+H]⁺.

### Example 218 was Prepared Following Scheme 210

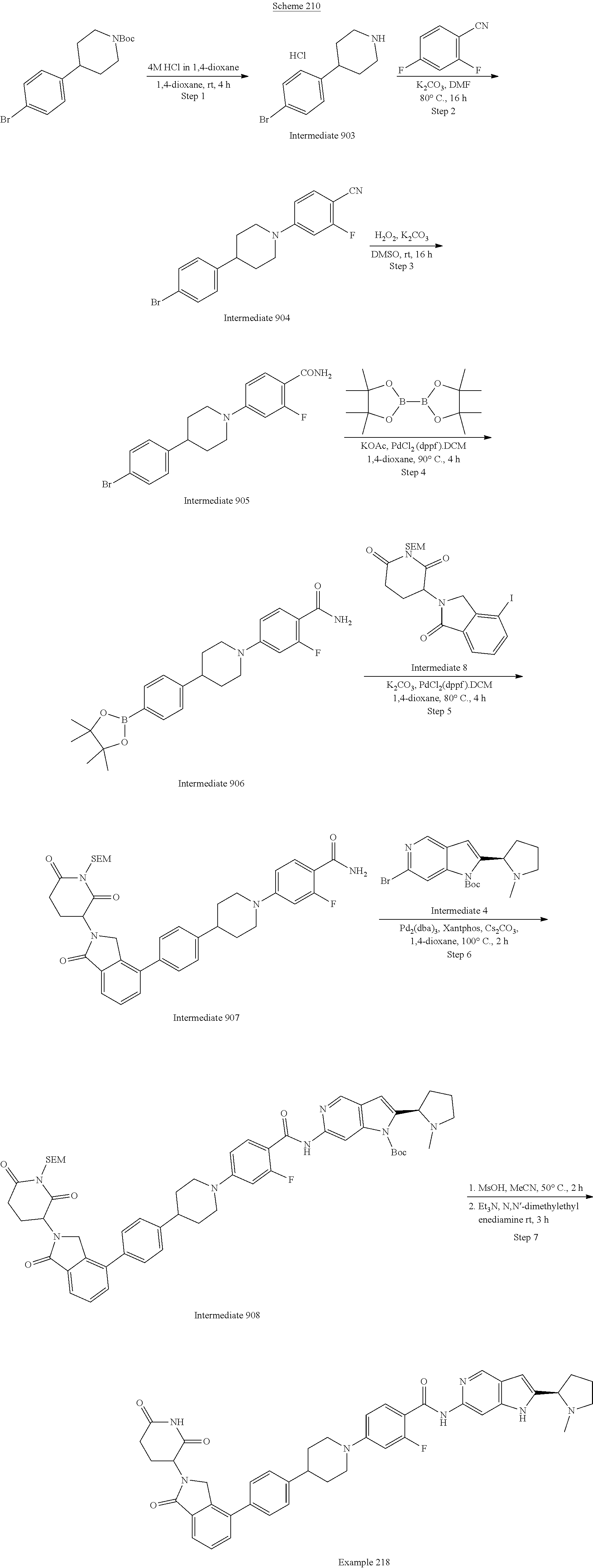

### Step 1

### Intermediate 903: 4-(4-bromophenyl)piperidine hydrochloride

To a cooled (0° C.) solution of tert-butyl 4-(4-bromophenyl)piperidine-1-carboxylate (CAS No. 769944-78-7, 3.0 g, 8.82 mmol) in 1,4-dioxane (10 mL) was added 4M hydrochloric acid in 1,4-dioxane (10 mL, 40.0 mmol). The reaction mixture was stirred at room temperature for 4 h then concentrated in vacuo to afford 4-(4-bromophenyl)piperidine hydrochloride (Intermediate 903, 2.0 g, 95%) as a white solid, which was used for the next step without further purification. ¹H NMR (400 MHz, DMSO-d₆) δ ppm 1.81-1.91 (m, 4H), 2.51-2.56 (m, 1H) 2.79-2.86 (m, 1H), 2.94-3.00 (m, 2H), 3.34-3.37 (m, 1H), 7.19 (d, J=8.22 Hz, 2H), 7.53 (d, J=8.22 Hz, 2H), 9.05 (br s, 1H). Mass spec m/z 241.9 [M+H+2]+.

### Step 2

### Intermediate 904: 4-(4-(4-bromophenyl)piperidin-1-yl)-2-fluorobenzonitrile

To a solution of 4-(4-bromophenyl)piperidine hydrochloride (Intermediate 903, 2.0 g, 8.32 mmol) and 2,4-difluorobenzonitrile (CAS No. 939-09-1, 1.39 g, 9.99 mmol) in dimethylformamide (10 mL) was added potassium carbonate (1.72 g, 12.49 mmol). The reaction mixture was stirred at 80° C. for 16 h. The reaction mixture was diluted with water (100 mL), the resultant precipitate was collected by filtration, washed with water (30 mL) and dried in vacuo. The crude material was purified by combi-flash chromatography, by eluting with 30% ethyl acetate in heptane, to afford 4-(4-(4-bromophenyl)piperidin-1-yl)-2-fluorobenzonitrile (Intermediate 904, 1.0 g, 33%) as an off-white solid. ¹H NMR (400 MHz, DMSO-d₆) δ ppm 1.55-1.65 (m, 2H), 1.81-1.85 (m, 2H), 2.79-2.84 (m, 1H), 2.96-3.03 (m, 2H), 4.08-4.11 (m, 2H), 6.86-6.89 (m, 1H), 6.95-6.99 (m, 1H), 7.22 (d, J=8.40 HZ, 2H), 7.48 (d, J=8.40 Hz, 2H), 7.57 (t, J=8.40 Hz, 1H). Mass spec m/z 361.0 [M+H]⁺.

### Step 3

### Intermediate 905: 4-(4-(4-bromophenyl)piperidin-1-yl)-2-fluorobenzamide

To a cooled (0° C.) solution of 4-(4-(4-bromophenyl)piperidin-1-yl)-2-fluorobenzonitrile (Intermediate 904, 1.0 g, 2.78 mmol) in dimethyl sulfoxide (10 mL) was added potassium carbonate (0.57 g, 4.18 mmol) and 30% hydrogen peroxide (0.17 mL, 5.57 mmol). The reaction mixture was stirred at room temperature for 16 h. The reaction mixture was diluted with water (100 mL), the resultant precipitate was collected by filtration and dried in vacuo to afford 4-(4-(4-bromophenyl)piperidin-1-yl)-2-fluorobenzamide (Intermediate 905, 1.0 g, 100%) as an off-white solid, which was used for the next step without further purification. ¹H NMR (400 MHz, DMSO-d₆) δ ppm 1.59-1.69 (m, 2H), 1.80-1.85 (m, 2H), 2.72-2.79 (m, 1H), 2.87-2.93 (m, 2H), 3.96-4.02 (m, 2H), 6.73-6.83 (m, 2H), 7.17 (br s, 1H), 7.23 (d, J=8.40 Hz, 2H), 7.30 (br s, 1H), 7.48 (d, J=8.40 Hz, 2H), 7.62 (t, J=9.05 Hz, 1H). Mass spec m/z 377.1 [M+H]⁺.

### Step 4

### Intermediate 906: 2-fluoro-4-(4-(4-(4,4,5,5-tetramethyl-1,3,2-dioxaborolan-2-yl)phenyl)piperidin-1-yl)benzamide

To a solution of 4-(4-(4-bromophenyl)piperidin-1-yl)-2-fluorobenzamide (Intermediate 905, 1.0 g, 2.7 mmol) and 4,4,4′,4′,5,5,5′,5′-octamethyl-2,2′-bi-1,3,2-dioxaborolane (CAS No. 73183-34-3, 0.09 g, 0.34 mmol) in 1,4-dioxane (5 mL) was added potassium acetate (0.79 g, 8.0 mmol). The reaction mixture was purged with argon for 15 min then PdCl₂(dppf) DCM (0.19 g, 0.27 mmol) was added. The reaction mixture was purged with argon for another 10 min and stirred at 90° C. for 4 h. The reaction mixture was concentrated in vacuo and the crude residue was triturated with heptane followed by n-pentane to afford 2-fluoro-4-(4-(4-(4,4,5,5-tetramethyl-1,3,2-dioxaborolan-2-yl)phenyl)piperidin-1-yl)benzamide (Intermediate 906, 1.70 g) as a brown solid which was carried forward into next step without purification. Mass spec m/z 425.1 [M+H]⁺.

### Step 5

### Intermediate 907: 4-(4-(4-(2-(2,6-dioxo-1-((2-(trimethylsilyl)ethoxy)methyl)piperidin-3-yl)-1-oxoisoindolin-4-yl)phenyl)piperidin-1-yl)-2-fluorobenzamide

To a solution of 2-fluoro-4-(4-(4-(4,4,5,5-tetramethyl-1,3,2-dioxaborolan-2-yl)phenyl)piperidin-1-yl)benzamide (Intermediate 906, 1.69 g, 3.99 mmol) and 3-(4-iodo-1-oxoisoindolin-2-yl)-1-((2-(trimethylsilyl)ethoxy)methyl)piperidine-2,6-dione (Intermediate 8, 1.0 g, 1.99 mmol) in 1,4-dioxane (20 mL) was added potassium carbonate (0.41 g, 2.99 mmol). The reaction mixture was purged with argon for 15 min then PdCl₂(dppf) DCM (0.14 g, 0.19 mmol) was added. The reaction mixture was purged with argon for another 10 min and stirred at 80° C. for 4 h. The reaction mixture was concentrated in vacuo and the crude material purified by combi-flash chromatography, by eluting with 40% ethyl acetate in heptane, to afford 4-(4-(4-(2-(2,6-dioxo-1-((2-(trimethylsilyl)ethoxy)methyl)piperidin-3-yl)-1-oxoisoindolin-4-yl)phenyl)piperidin-1-yl)-2-fluorobenzamide (Intermediate 907, 1.2 g, 90%) as an off-white solid. ¹H NMR (400 MHz, DMSO-d₆) δ ppm −0.10 (s, 9H), 0.77-0.82 (m, 3H), 1.19-1.22 (m, 2H), 1.68-1.73 (m, 2H), 1.84-1.87 (m, 2H), 1.96-2.04 (m, 1H), 2.29-2.41 (m, 1H), 2.81-3.00 (m, 3H), 3.41-3.53 (m, 3H), 3.97-4.02 (m, 2H), 4.96-5.03 (m, 2H), 5.23-5.26 (m, 1H), 6.72-8.83 (m, 2H), 7.13 (br s, 1H), 7.28 (br s, 1H), 7.33-7.36 (m, 2H), 7.44-7.56 (m, 2H), 7.59-7.69 (m, 3H), 7.73-7.80 (m, 1H). Mass spec m/z 671.3 [M+H]⁺.

### Step 6

### Intermediate 908: tert-butyl 6-(4-(4-(4-(2-(2,6-dioxo-1-((2-(trimethylsilyl)ethoxy)methyl)piperidin-3-yl)-1-oxoisoindolin-4-yl)phenyl)piperidin-1-yl)-2-fluorobenzamido)-2-((R)-1-methylpyrrolidin-2-yl)-1H-pyrrolo[3,2-c]pyridine-1-carboxylate

To a solution of 4-(4-(4-(2-(2,6-dioxo-1-((2-(trimethylsilyl) ethoxy)methyl)piperidin-3-yl)-1-oxoisoindolin-4-yl)phenyl)piperidin-1-yl)-2-fluorobenzamide (Intermediate 907, 0.70 g, 1.05 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.50 g, 1.31 mmol) followed by cesium carbonate (1.29 g, 3.94 mmol). The reaction mixture was purged with argon for 15 min then Pd₂(dba)₃ (0.18 g, 0.19 mmol) and Xantphos (0.23 g, 0.39 mmol) were added. The reaction mixture was purged with argon for another 10 min and stirred at 100° C. for 2 h. The reaction mixture was concentrated in vacuo and the crude material was purified by combi-flash chromatography, by eluting with 90% ethyl acetate in heptane, to afford tert-butyl 6-(4-(4-(4-(2-(2,6-dioxo-1-((2-(trimethylsilyl)ethoxy)methyl)piperidin-3-yl)-1-oxoisoindolin-4-yl)phenyl)piperidin-1-yl)-2-fluorobenzamido)-2-((R)-1-methylpyrrolidin-2-yl)-1H-pyrrolo[3,2-c]pyridine-1-carboxylate (Intermediate 908, 0.88 g, 69%) as a white solid. ¹H NMR (400 MHz, DMSO-d₆) δ ppm −0.07 (s, 9H), 0.78-0.81 (m, 2H), 1.15-1.18 (m, 1H), 1.70 (s, 9H), 1.72-1.79 (m, 3H), 1.89-1.93 (m, 2H), 2.00-2.07 (m, 1H), 2.35 (s, 3H), 2.36-2.41 (m, 3H), 2.73-2.81 (m, 1H), 2.83-2.92 (m, 1H), 2.95-3.15 (m, 4H), 3.45-3.54 (m, 2H), 3.88-3.95 (m, 1H), 4.00-4.04 (m, 1H), 4.06-4.13 (m, 2H), 4.31-4.35 (m, 1H), 4.65-4.69 (m, 1H), 4.99-5.08 (m, 2H), 5.26-5.31 (m, 1H), 6.73 (s, 1H), 6.83-6.92 (m, 2H), 7.39 (d, J=8.25 Hz, 2H), 7.53 (d, J=8.25 Hz, 2H), 7.65-7.76 (m, 4H), 8.54 (s, 1H), 8.93 (s, 1H), 9.78 (br s, 1H). Mass spec m/z 970.5 [M+H]⁺.

### Step 7

### Example 218: 4-(4-(4-(2-(2,6-dioxopiperidin-3-yl)-1-oxoisoindolin-4-yl)phenyl)piperidin-1-yl)-2-fluoro-N-(2-((R)-1-methylpyrrolidin-2-yl)-1H-pyrrolo[3,2-c]pyridin-6-yl)benzamide

To a solution of tert-butyl 6-(4-(4-(4-(2-(2,6-dioxo-1-((2-(trimethylsilyl)ethoxy)methyl)piperidin-3-yl)-1-oxoisoindolin-4-yl)phenyl)piperidin-1-yl)-2-fluorobenzamido)-2-((R)-1-methylpyrrolidin-2-yl)-1H-pyrrolo[3,2-c]pyridine-1-carboxylate (Intermediate 908, 0.4 g, 0.4 mmol) in acetonitrile (5 mL) was added methanesulfonic acid (0.4 mL, 6.0 mmol) and the reaction mixture was heated at 50° C. for 2 h. The reaction mixture was cooled to room temperature, then N,N′-dimethylethylenediamine (0.31 mL, 2.0 mmol followed by triethylamine (0.8 g, 8.0 mmol were added and the mixture was stirred at room temperature for 3 h. The reaction mixture was diluted with water (50 mL). The resultant precipitate was collected by filtration and dried in vacuo. The crude solid was purified by preparative HPLC (Method A) to afford 4-(4-(4-(2-(2,6-dioxopiperidin-3-yl)-1-oxoisoindolin-4-yl)phenyl)piperidin-1-yl)-2-fluoro-N-(2-((R)-1-methylpyrrolidin-2-yl)-1H-pyrrolo[3,2-c]pyridin-6-yl)benzamide (Example 218, 0.043 g, 10%) as an off-white solid. ¹H NMR (400 MHz, DMSO-d₆) δ ppm 1.86-1.99 (m, 8H), 2.10-2.15 (m, 1H), 2.16 (s, 3H), 2.23-2.30 (m, 1H), 2.42-2.46 (m, 1H), 2.54-2.60 (m, 1H), 2.84-2.93 (m, 2H), 2.97-3.03 (m, 2H), 3.12-3.16 (m, 2H), 4.08-4.11 (m, 2H), 4.40-4.44 (m, 1H), 4.60-4.64 (m, 1H), 5.11-5.16 (m, 1H), 6.38 (s, 1H), 6.86-6.94 (m, 2H), 7.41 (d, J=8.25 Hz, 2H), 7.55 (d, J=8.25 Hz, 2H), 7.63 (t, J=7.60 Hz, 1H), 7.68-7.74 (m, 1H), 7.75-7.79 (m, 2H), 8.21 (s, 1H), 8.45 (br s, 1H), 9.52 (d, J=9.60 Hz, 1H), 10.96 (br s, 1H), 11.37 (br s, 1H). Mass spec m/z 740.0 [M+H]⁺.

### Example 219 was Synthesised Following Scheme 211

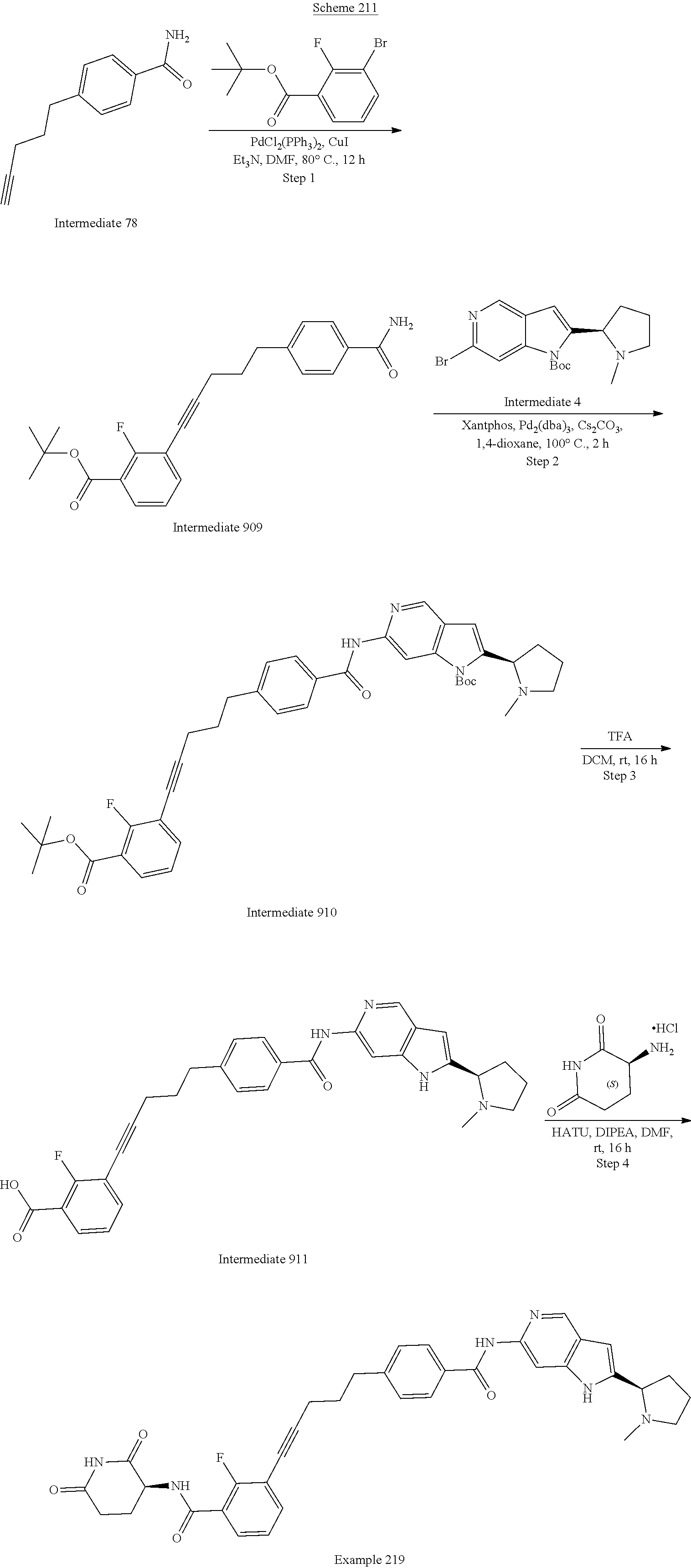

### Step 1

### Intermediate 909: tert-butyl 3-(5-(4-carbamoylphenyl)pent-1-yn-1-yl)-2-fluorobenzoate

To a solution of tert-butyl 3-bromo-2-fluorobenzoate (CAS No: 1262834-58-1, 1.0 g, 3.60 mmol) in dimethylformamide (1.0 mL) was added 4-(pent-4-yn-1-yl)benzamide (Intermediate 78, 1.03 g, 5.50 mmol) followed by triethylamine (3.50 mL, 25.20 mmol). The reaction mixture was purged with argon for 15 min then copper (I) iodide (0.10 g, 0.55 mmol) and PdCl₂(PPh₃)₂ (0.25 g, 0.36 mmol) were added. The reaction mixture was purged with argon for a further 10 min and then stirred at 80° C. for 12 h. After completion, the reaction mixture was diluted with water (50 mL) and extracted with ethyl acetate (2×100 mL). The combined organic layers were dried over anhydrous Na₂SO₄ and concentrated in vacuo to afford tert-butyl 3-(5-(4-carbamoylphenyl)pent-1-yn-1-yl)-2-fluorobenzoate (Intermediate 909, 1.20 g) as a yellow semi-solid which was used into next step without purification. Mass spec m/z 380.0 [M+H]⁺.

### Step 2

### Intermediate 910: tert-butyl (R)-6-(4-(5-(3-(tert-butoxycarbonyl)-2-fluorophenyl)pent-4-yn-1-yl)benzamido)-2-(1-methylpyrrolidin-2-yl)-1H-pyrrolo[3,2-c]pyridine-1-carboxylate

To a solution tert-butyl 3-(5-(4-carbamoylphenyl)pent-1-yn-1-yl)-2-fluorobenzoate (Intermediate 909, 0.450 g, 1.18 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.45 g, 1.30 mmol) followed by cesium carbonate (1.15 g, 3.50 mmol). The reaction mixture was purged with argon for 15 min then Pd₂(dba)₃ (0.16 g, 0.18 mmol) and Xantphos (0.11 g, 0.18 mmol) were added. The reaction mixture was purged with argon for another 10 min and then stirred at 100° C. for 2 h. After completion, the reaction mixture was concentrated in vacuo and the crude material was purified by combi-flash chromatography, by eluting with 80-90% ethyl acetate in heptane, to afford tert-butyl (R)-6-(4-(5-(3-(tert-butoxycarbonyl)-2-fluorophenyl)pent-4-yn-1-yl)benzamido)-2-(1-methylpyrrolidin-2-yl)-1H-pyrrolo[3,2-c]pyridine-1-carboxylate (Intermediate 910, 0.53 g, 66%) as an off-white solid. ¹H NMR (400 MHz, DMSO-d₆) δ ppm 1.54 (s, 9H), 1.59-1.65 (m, 2H), 1.70 (s, 9H), 1.74-1.76 (m, 2H), 1.90-1.93 (m, 2H), 2.31-2.33 (m, 2H), 2.35 (s, 3H), 2.51-2.53 (m, 1H), 2.83 (t, J=7.60 Hz, 2H), 3.12-3.15 (m, 1H), 3.90-3.93 (m, 1H), 6.75 (s, 1H), 7.28 (t, J=7.60 Hz, 1H), 7.38 (d, J=8.40 Hz, 2H), 7.70-7.78 (m, 2H), 8.00 (d, J=8.0, 2H), 8.60 (s, 1H), 8.93 (s, 1H), 10.65 (s, 1H). Mass spec m/z 681.3 [M+H]⁺.

### Step 3

### Intermediate 911: (R)-2-fluoro-3-(5-(4-((2-(1-methylpyrrolidin-2-yl)-1H-pyrrolo[3,2-c]pyridin-6-yl)carbamoyl)phenyl)pent-1-yn-1-yl)benzoic acid

To a solution of tert-butyl (R)-6-(4-(5-(3-(tert-butoxycarbonyl)-2-fluorophenyl)pent-4-yn-1-yl)benzamido)-2-(1-methylpyrrolidin-2-yl)-1H-pyrrolo[3,2-c]pyridine-1-carboxylate (Intermediate 910, 0.450 g, 0.66 mmol) in dichloromethane (4.5 mL) was added trifluoroacetic acid (4.5 mL, 58.0 mm) and the reaction mixture was stirred at room temperature for 16 h. After completion, the reaction mixture was concentrated in vacuo. The crude material was triturated with diethyl ether (2×10 mL) and dried in vacuo to afford (R)-2-fluoro-3-(5-(4-((2-(1-methylpyrrolidin-2-yl)-1H-pyrrolo[3,2-c]pyridin-6-yl)carbamoyl)phenyl)pent-1-yn-1-yl)benzoic acid (Intermediate 911, 0.5 g), as a red semi-solid which was taken forward to next reaction as such without any further purification. Mass spec m/z 525.2 [M+H]⁺.

### Step 4

### Example 219: N—((S)-2,6-dioxopiperidin-3-yl)-2-fluoro-3-(5-(4-((2-((R)-1-methylpyrrolidin-2-yl)-1H-pyrrolo[3,2-c]pyridin-6-yl)carbamoyl)phenyl)pent-1-yn-1-yl)benzamide

To a cooled (0° C.) solution of (R)-2-fluoro-3-(5-(4-((2-(1-methylpyrrolidin-2-yl)-1H-pyrrolo[3,2-c]pyridin-6-yl)carbamoyl)phenyl)pent-1-yn-1-yl)benzoic acid (Intermediate 911, 0.45 g, 0.71 mmol) in dimethylformamide (5 mL) was added HATU (0.42 g, 1.10 mmol) and N,N-diisopropylethylamine (0.6 mL, 3.52 mmol) followed by the addition of (S)-3-aminopiperidine-2,6-dione hydrochloride (CAS No: 25181-50-4, 0.21 g, 1.06 mmol) and the reaction mixture was stirred at room temperature for 3 h. After completion, the reaction mixture was poured into ice-cold water (150 mL), the resultant precipitate was collected by filtration and dried in vacuo. The crude material was purified by preparative HPLC (Method A) to afford N—((S)-2,6-dioxopiperidin-3-yl)-2-fluoro-3-(5-(4-((2-((R)-1-methylpyrrolidin-2-yl)-1H-pyrrolo[3,2-c]pyridin-6-yl)carbamoyl)phenyl)pent-1-yn-1-yl)benzamide (Example 219, 0.185 g, 41%) as an off-white solid. ¹H NMR (400 MHz, DMSO-d₆) δ ppm 1.78-1.95 (m, 5H), 2.01-2.09 (m, 2H), 2.11-2.14 (m, 1H), 2.16 (s, 3H), 2.23-2.27 (m, 1H), 2.52-2.56 (m, 2H), 2.75-2.85 (m, 3H), 3.12-3.16 (m, 1H), 3.28-3.30 (m, 1H), 3.31-3.33 (m, 1H), 4.76-4.79 (m, 1H), 6.85 (s, 1H), 7.28 (t, J=7.60 Hz, 1H), 7.37 (d, J=8.40 Hz, 2H), 7.58-7.65 (m, 2H), 8.00 (d, J=8.40 Hz, 2H), 8.20 (s, 1H), 8.50 (s, 1H), 8.70-8.72 (m, 1H), 10.37 (s, 1H), 11.90 (s, 1H), 11.36 (s, 1H). Mass spec m/z 635.2 [M+H]⁺.

### Example 220 was Prepared Following Scheme 212

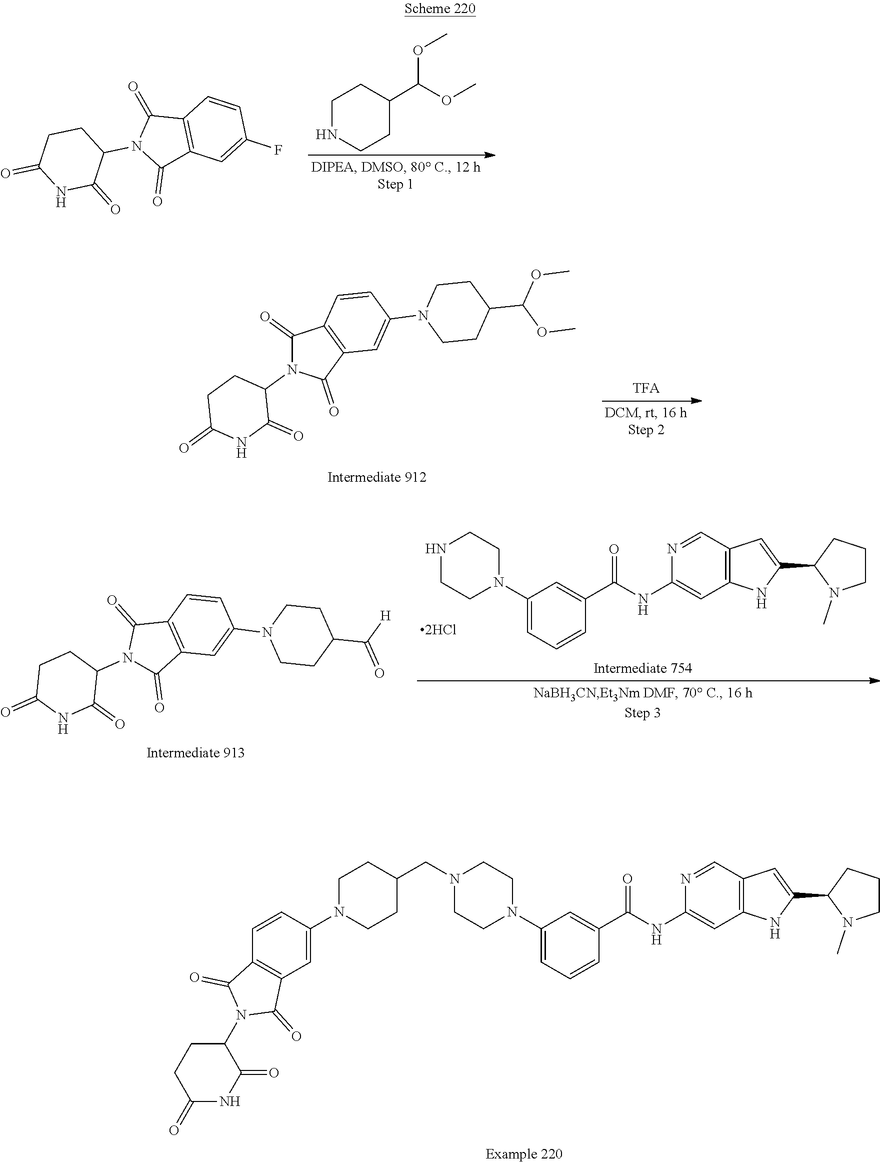 [SMILES: O=C1CCC(N2C(=O)c3ccc(F)cc3C2=O)C(=O)N1 ; COC(OC)C1CCNCC1 ; COC(OC)C1CCN(c2ccc3c(c2)C(=O)N(C2CCC(=O)NC2=O)C3=O)CC1 ; O=CC1CCN(c2ccc3c(c2)C(=O)N(C2CCC(=O)NC2=O)C3=O)CC1 ; CN1CCC[C@@H]1c1cc2cnc(NC(=O)c3cccc(N4CCNCC4)c3)cc2[nH]1 ; CN1CCC[C@@H]1c1cc2cnc(NC(=O)c3cccc(N4CCN(CC5CCN(c6ccc7c(c6)C(=O)N(C6CCC(=O)NC6=O)C7=O)CC5)CC4)c3)cc2[nH]1]

### Step 1

### Intermediate 912: 5-(4-(dimethoxymethyl)piperidin-1-yl)-2-(2,6-dioxopiperidin-3-yl)isoindoline-1,3-dione

To a solution of 2-(2,6-dioxopiperidin-3-yl)-5-fluoroisoindoline-1,3-dione (CAS No. 835616-61-0, 2.0 g, 6.88 mmol) and 4-(dimethoxymethyl)piperidine (CAS No. 188646-83-5, 1.19 g, 10.3 mmol) in dimethyl sulfoxide (20 mL) was added N,N-diisopropylethylamine amine (2.72 g, 20.6 mmol) and the reaction mixture was heated at 80° C. for 12 h. The reaction mixture was diluted with water (200 mL) and extracted with DCM (2×100 mL). The combined organic layers were washed with water (150 mL), dried over Na₂SO₄ and concentrated in vacuo. The crude residue was purified by combi-flash chromatography, by eluting with 50% ethyl acetate in heptane, to afford 5-(4-(dimethoxymethyl)piperidin-1-yl)-2-(2,6-dioxopiperidin-3-yl)isoindoline-1,3-dione (Intermediate 912, 0.9 g, 32%) as an off-white solid. ¹H NMR (400 MHz, DMSO-d₆) δ ppm 1.24-1.32 (m, 2H), 1.68-1.73 (m, 2H), 1.85-1.92 (m, 1H), 199-2.04 (m, 1H), 2.54-2.62 (m, 2H), 2.84-3.01 (m, 3H), 3.31 (s, 6H), 4.04-4.10 (m, 3H), 5.03-5.10 (m, 1H), 7.20 (d, J=8.40 Hz, 1H), 7.30 (s, 1H), 7.65 (d, J=8.0 Hz, 1H), 11.06 (br s, 1H). Mass spec m/z 416.2 [M+H]⁺.

### Step 2

### Intermediate 913: 1-(2-(2,6-dioxopiperidin-3-yl)-1,3-dioxoisoindolin-5-yl)piperidine-4-carbaldehyde

To a cooled (0° C.) solution of 5-(4-(dimethoxymethyl)piperidin-1-yl)-2-(2,6-dioxopiperidin-3-yl)isoindoline-1,3-dione (Intermediate 912, 0.9 g, 2.17 mmol) in DCM (10 mL) was added trifluoroacetic acid (3.71 g, 32.50 mmol) and the reaction mixture was stirred at room temperature for 16 h. The reaction mixture was concentrated in vacuo to afford 1-(2-(2,6-dioxopiperidin-3-yl)-1,3-dioxoisoindolin-5-yl)piperidine-4-carbaldehyde (Intermediate 913, 0.72 g, 90%) as an off-white solid which was carried forward into next step without purification. Mass spec m/z 370.1 [M+H]⁺.

### Step 3

### Example 220: 3-(4-((1-(2-(2,6-dioxopiperidin-3-yl)-1,3-dioxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide

To a solution of 1-(2-(2,6-dioxopiperidin-3-yl)-1,3-dioxoisoindolin-5-yl)piperidine-4-carbaldehyde (Intermediate 913, 0.3 g, 0.81 mmol) in dimethylformamide (5 mL) was added a solution of (R)—N-(2-(1-methylpyrrolidin-2-yl)-1H-pyrrolo[3,2-c]pyridin-6-yl)-3-(piperazin-1-yl)benzamide dihydrochloride (Intermediate 754, 3.73 g, 8.46 mmol) in dimethylformamide (5 mL) followed by triethylamine (0.33 g, 3.25 mmol) and sodium cyanoborohydride (0.11 g, 1.62 mmol) and the reaction mixture was heated at 70° C. for 16 h. The reaction mixture was quenched with ice cold water (100 mL) and the resultant precipitate was collected by filtration, washed with heptane (200 mL), and dried in vacuo. The crude solid was purified by preparative HPLC (Method A) to afford 3-(4-((1-(2-(2,6-dioxopiperidin-3-yl)-1,3-dioxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide (Example 220, 0.02 g, 3%) as a pale yellow solid. ¹H NMR (400 MHz, DMSO-d₆) δ ppm 1.12-1.24 (m, 2H), 1.75-1.92 (m, 6H), 1.94-2.05 (m, 1H), 2.16 (s, 3H), 2.18-2.01 (m, 1H), 2.03-2.29 (m, 3H), 2.44-2.49 (m, 6H), 2.55-2.59 (m, 1H), 2.82-2.89 (m, 1H), 2.93-3.03 (m, 2H), 3.09-3.18 (m, 1H), 3.21-3.29 (m, 4H), 4.04-4.11 (m, 2H), 5.04-5.09 (m, 1H), 6.39 (s, 1H), 7.12 (d, J=6.80 Hz, 1H), 7.22-7.25 (m, 1H), 7.32 (t, J=8.40 Hz, 2H), 7.44 (d, J=7.60 Hz, 1H), 7.58-7.62 (m, 1H), 7.65 (d, J=8.40 Hz, 1H), 8.19 (s, 1H), 8.50 (s, 1H), 10.43 (s, 1H), 11.07 (s, 1H), 11.36 (s, 1H). Mass spec m/z 758.0 [M+H]⁺.

### Example 221 was Prepared Following Scheme 213

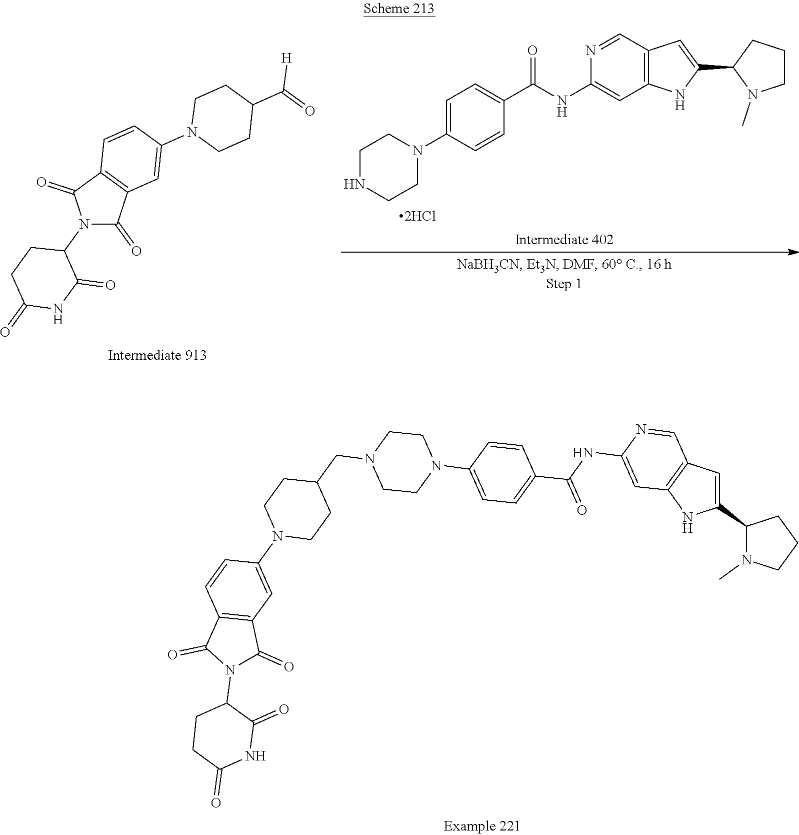 [SMILES: O=CC1CCN(c2ccc3c(c2)C(=O)N(C2CCC(=O)NC2=O)C3=O)CC1 ; CN1CCC[C@@H]1c1cc2cnc(NC(=O)c3ccc(N4CCNCC4)cc3)cc2[nH]1 ; CN1CCC[C@@H]1c1cc2cnc(NC(=O)c3ccc(N4CCN(CC5CCN(c6ccc7c(c6)C(=O)N(C6CCC(=O)NC6=O)C7=O)CC5)CC4)cc3)cc2[nH]1]

### Step 1

### Example 221: 4-(4-((1-(2-(2,6-dioxopiperidin-3-yl)-1,3-dioxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide

To a solution of 1-(2-(2,6-dioxopiperidin-3-yl)-1,3-dioxoisoindolin-5-yl)piperidine-4-carbaldehyde (Intermediate 913, 0.3 g, 0.81 mmol) in dimethylformamide (5 mL) was added (R)—N-(2-(1-methylpyrrolidin-2-yl)-1H-pyrrolo[3,2-c]pyridin-6-yl)-4-(piperazin-1-yl)benzamide dihydrochloride (Intermediate 402, 0.36 g, 0.81 mmol), triethylamine (0.33 g, 3.25 mmol) and sodium cyanoborohydride (0.15 g, 2.43 mmol) and the reaction mixture was heated at 60° C. for 16 h. The reaction mixture was diluted with water (100 mL) and extracted with diethyl ether (3×120 mL). The combined organic layers were dried over Na₂SO₄ and concentrated in vacuo. The crude material was purified by preparative HPLC (Method A) to afford 4-(4-((1-(2-(2,6-dioxopiperidin-3-yl)-1,3-dioxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide (Example 221, 0.01 g, 2%) as a pale yellow solid. ¹H NMR (400 MHz, DMSO-d₆) δ ppm 1.19-1.24 (m, 2H), 1.75-2.00 (m, 6H), 2.02-2.05 (m, 1H), 2.16 (s, 3H), 2.17-2.19 (m, 1H), 2.01-2.29 (m, 3H), 2.38-2.43 (m, 3H), 2.51-2.57 (m, 3H), 2.58-2.62 (m, 1H), 2.83-2.88 (m, 1H), 2.89-2.92 (m, 2H), 3.10-3.17 (m, 1H), 3.28-3.33 (m, 4H), 4.04-4.10 (m, 2H), 5.03-5.10 (m, 1H), 6.37 (s, 1H), 6.99 (d, J=8.80 Hz, 2H), 7.24 (dd, J=6.40, 2.0 Hz, 1H), 7.29-7.32 (m, 1H), 7.65 (d, J=8.4 Hz, 1H), 7.96 (d, J=8.80 Hz, 2H), 8.17 (s, 1H), 8.48 (s, 1H), 10.90 (s, 1H), 11.10 (s, 1H), 11.32 (s, 1H). Mass spec m/z 758.0 [M+H]⁺.

### Example 222 was Prepared Following Scheme 214

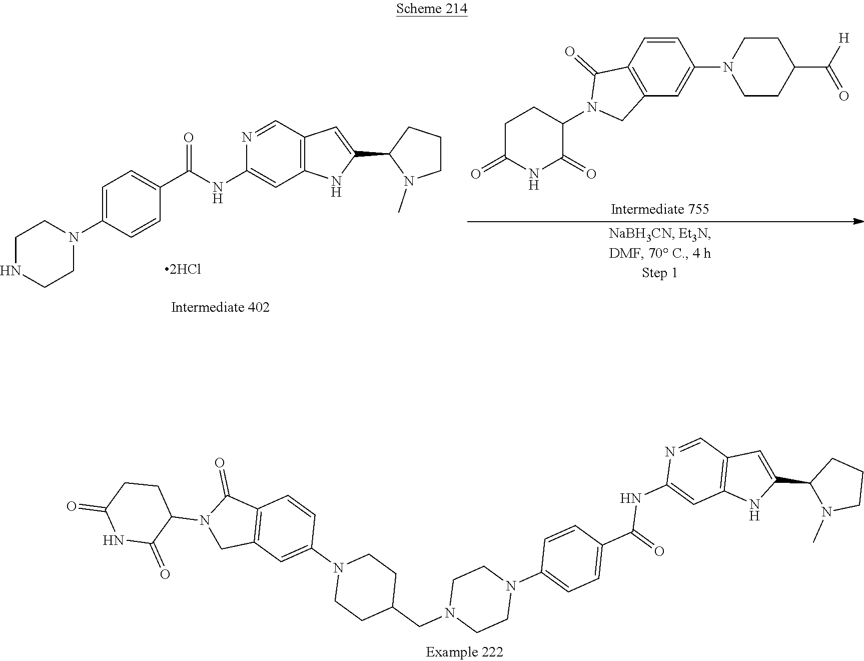

### Step 1

### Example 222: 4-(4-((1-(2-(2,6-dioxopiperidin-3-yl)-1-oxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide

To a solution of 1-(2-(2,6-dioxopiperidin-3-yl)-1-oxoisoindolin-5-yl)piperidine-4-carbaldehyde (Intermediate 755, 0.5 g, 1.41 mmol) in dimethylformamide (5 mL) was added (R)—N-(2-(1-methylpyrrolidin-2-yl)-1H-pyrrolo[3,2-c]pyridin-6-yl)-4-(piperazin-1-yl)benzamide dihydrochloride (Intermediate 402, 0.62 g, 1.41 mmol), triethylamine (0.79 mL, 5.63 mmol) and sodium cyanoborohydride (0.27 g, 4.22 mmol) and the reaction mixture was heated at 60° C. for 4 h. The reaction mixture was diluted with water (100 mL), the resultant precipitate was collected by filtration and dried in vacuo. The crude material was purified by preparative HPLC (Method A) to afford 4-(4-((1-(2-(2,6-dioxopiperidin-3-yl)-1-oxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide (Example 222, 0.1 g, 9%) as an off-white solid. ¹H NMR (400 MHz, DMSO-d₆) δ ppm 1.19-1.23 (m, 2H), 1.80-1.95 (m, 8H), 2.17 (s, 3H), 2.21-2.26 (m, 2H), 2.32-2.34 (m, 1H), 2.48-2.49 (m, 1H), 2.51-2.56 (m, 1H), 2.61-2.63 (m, 1H), 2.82-2.90 (m, 3H), 3.12-3.16 (m, 2H), 3.28-3.31 (m, 7H), 3.87-3.90 (m, 2H), 4.18-4.22 (m, 1H), 4.30-4.34 (m, 1H), 5.02-5.07 (m, 1H), 6.38 (s, 1H), 6.98 (d, J=9.20 Hz, 2H), 7.04-7.06 (m, 2H), 7.50 (d, J=8.80 Hz, 1H), 7.96 (d, J=8.80 Hz, 2H), 8.15 (s, 1H), 8.48 (s, 1H), 10.10 (s, 1H), 10.94 (s, 1H), 11.33 (s, 1H). Mass spec m/z 744.0 [M+H]⁺.

### Example 223 was Synthesised Following Scheme 215

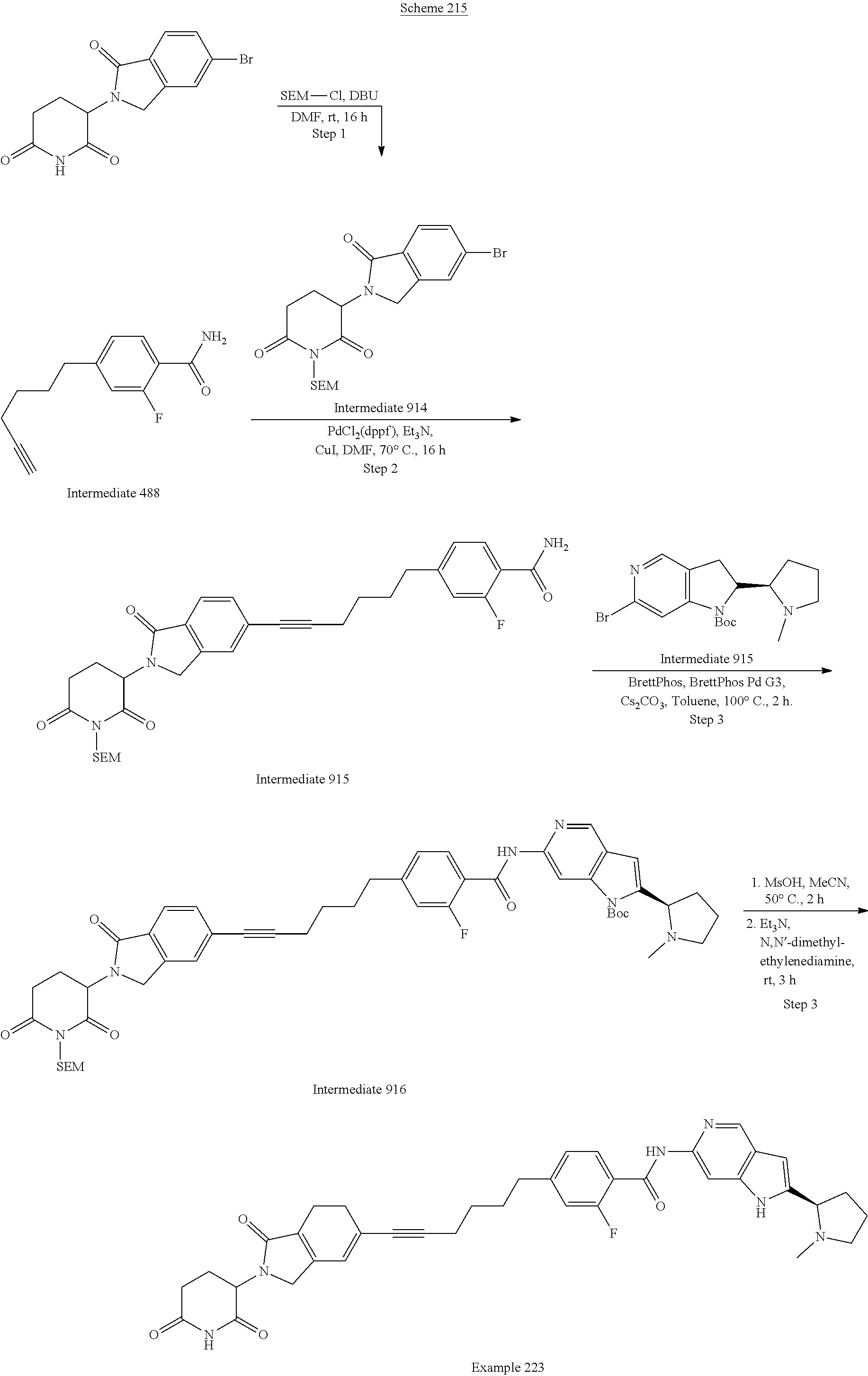

### Step 1

### Intermediate 914: 3-(5-bromo-1-oxoisoindolin-2-yl)-1-((2-(trimethylsilyl)ethoxy)methyl)piperidine-2,6-dione

To a cooled (0° C.) solution of 3-(5-bromo-1-oxoisoindolin-2-yl)piperidine-2,6-dione (CAS No: 1010100-26-1, 3.0 g, 9.29 mmol) in dimethylformamide (10 mL) was added 1,8-diazabicyclo[5.4.0]undec-7-ene (4.28 mL, 27.9 mmol) and the reaction mixture was stirred at room temperature for 10 min. (2-(chloromethoxy)ethyl)trimethylsilane (5.1 mL, 27.9 mmol) was then added dropwise and the reaction mixture was stirred at room temperature for 16 h. After completion, the reaction mixture was quenched with ice cold water (100 mL) and extracted with ethyl acetate (2×100 mL). The combined organic layers were dried over anhydrous Na₂SO₄, filtered, and concentrated in vacuo. The crude material was purified by combi-flash chromatography, by eluting with 50% ethyl acetate in heptane, to afford 3-(5-bromo-1-oxoisoindolin-2-yl)-1-((2-(trimethylsilyl)ethoxy)methyl)piperidine-2,6-dione (Intermediate 914, 1.5 g, 36%) as a white solid. ¹H NMR (400 MHz, DMSO-d₆) δ ppm −0.02 (s, 9H), 0.81-0.85 (m, 2H), 2.04-2.10 (m, 1H), 2.33-2.41 (m, 1H), 2.75-2.83 (m, 1H), 3.00-3.10 (m, 1H), 3.51-3.53 (m, 2H), 4.27-4.52 (m, 2H), 5.01-5.08 (m, 2H), 5.20-5.25 (m, 1H), 7.64-7.76 (m, 2H), 7.90 (s, 1H). Mass spec m/z 453.0 [M+H]⁺.

### Step 2

### Intermediate 915: 4-(6-(2-(2,6-dioxo-1-((2-(trimethylsilyl)ethoxy)methyl)piperidin-3-yl)-1-oxoisoindolin-5-yl) hex-5-yn-1-yl)-2-fluorobenzamide

To a solution of 2-fluoro-4-(hex-5-yn-1-yl)benzamide (Intermediate 488, 0.3 g, 1.37 mmol) in dimethylformamide (3 mL) was added 3-(5-bromo-1-oxoisoindolin-2-yl)-1-((2-(trimethylsilyl)ethoxy)methyl)piperidine-2,6-dione (Intermediate 914, 0.25 g, 0.55 mmol) followed by triethylamine (0.3 mL, 1.65 mmol). The reaction mixture was purged with argon for 15 min then copper (I) iodide (0.01 g, 0.08 mmol) and PdCl₂(dppf) (0.02 g, 0.02 mmol) were added. The reaction mixture was further purged with argon for 10 min and heated at 70° C. for 16 h. After completion, the reaction mixture was poured into ice cold water (50 mL) and extracted with ethyl acetate (3×80 mL). The combined organic layers were dried over anhydrous Na₂SO₄ and concentrated in vacuo. The crude material was purified by combi-flash chromatography, by eluting with 5% MeOH in DCM, to afford 4-(6-(2-(2,6-dioxo-1-((2-(trimethylsilyl)ethoxy)methyl)piperidin-3-yl)-1-oxoisoindolin-5-yl)hex-5-yn-1-yl)-2-fluorobenzamide (Intermediate 915, 0.25 g, 77%) as a yellow solid. ¹H NMR (400 MHz, DMSO-d₆) δ ppm −0.03 (s, 9H), 0.84-0.87 (m, 2H), 1.42-1.48 (m, 1H), 1.55-1.66 (m, 3H), 1.71-1.77 (m, 2H), 2.03-2.07 (m, 1H), 2.29-2.42 (m, 2H), 2.61-2.67 (m, 1H), 2.69 (t, J=7.40 Hz, 2H), 2.77-2.82 (m, 1H), 3.01-3.12 (m, 1H), 4.24-4.30 (m, 1H), 4.44-4.48 (m, 1H), 5.01-5.09 (m, 2H), 5.21-5.26 (m, 1H), 7.08-7.14 (m, 2H), 7.16 (br s, 1H), 7.51 (d, J=7.82 Hz, 1H), 7.57-7.61 (m, 2H), 7.63 (br s, 1H), 7.96 (s, 1H). Mass spec m/z 590.2 [M+H]⁺.

### Step 3

### Intermediate 916: tert-butyl 6-(4-(6-(2-(2,6-dioxo-1-((2-(trimethylsilyl)ethoxy)methyl)piperidin-3-yl)-1-oxoisoindolin-5-yl)hex-5-yn-1-yl)-2-fluorobenzamido)-2-((R)-1-methylpyrrolidin-2-yl)-1H-pyrrolo[3,2-c]pyridine-1-carboxylate

To a solution of 4-(6-(2-(2,6-dioxo-1-((2-(trimethylsilyl)ethoxy)methyl)piperidin-3-yl)-1-oxoisoindolin-5-yl)hex-5-yn-1-yl)-2-fluorobenzamide (Intermediate 915, 0.35 g, 0.59 mmol) in toluene (5 mL) was added tert-butyl (R)-6-bromo-2-(1-methylpyrrolidin-2-yl)-1H-pyrrolo[3,2-c]pyridine-1-carboxylate (Intermediate 4, 0.26 g, 0.79 mmol) followed by cesium carbonate (0.34 g, 1.75 mmol). The reaction mixture was purged with argon for 15 min, then BrettPhos Pd G3 (0.08 g, 0.08 mmol) and BrettPhos (0.06 g, 0.11 mmol) were added. The reaction mixture was further purged with argon for 10 min and heated at 100° C. for 2 h. After completion, the reaction mixture was concentrated in vacuo. The crude material was purified by combi-flash chromatography, by eluting with 5% MeOH in DCM, to afford tert-butyl 6-(4-(6-(2-(2,6-dioxo-1-((2-(trimethylsilyl)ethoxy)methyl)piperidin-3-yl)-1-oxoisoindolin-5-yl)hex-5-yn-1-yl)-2-fluorobenzamido)-2-((R)-1-methylpyrrolidin-2-yl)-1H-pyrrolo[3,2-c]pyridine-1-carboxylate (Intermediate 916, 0.30 g, 57%) as a yellow solid. ¹H NMR (400 MHz, DMSO-d₆) δ ppm −0.03 (s, 9H), 0.81-0.84 (m, 2H), 1.58-1.65 (m, 4H), 1.68 (s, 9H), 1.74-1.78 (m, 4H), 2.29-2.33 (m, 4H), 2.35 (s, 3H), 2.63-2.71 (m, 4H), 3.02-3.12 (m, 2H), 3.51-3.54 (m, 2H), 3.90-3.94, (m, 1H), 4.21-4.30 (m, 2H), 4.43-4.49 (m, 1H), 5.19-5.24 (m, 2H), 6.74 (s, 1H), 7.20 (t, J=8.40 Hz, 2H), 7.52-7.55 (m, 2H), 7.63 (s, 1H), 7.66-7.70 (m, 1H), 8.55 (s, 1H), 8.91 (s, 1H), 10.49 (br s, 1H). Mass spec m/z 891.4 [M+H]⁺.

### Step 4

### Example 223: 4-(6-(2-(2,6-dioxopiperidin-3-yl)-1-oxoisoindolin-5-yl)hex-5-yn-1-yl)-2-fluoro-N-(2-((R)-1-methylpyrrolidin-2-yl)-1H-pyrrolo[3,2-c]pyridin-6-yl)benzamide

To a solution of tert-butyl 6-(4-(6-(2-(2,6-dioxo-1-((2-(trimethylsilyl)ethoxy)methyl)piperidin-3-yl)-1-oxoisoindolin-5-yl)hex-5-yn-1-yl)-2-fluorobenzamido)-2-((R)-1-methylpyrrolidin-2-yl)-1H-pyrrolo[3,2-c]pyridine-1-carboxylate (Intermediate 916, 0.30 g, 0.33 mmol) in acetonitrile (5 mL) was added methanesulfonic acid (0.06 mL, 1.01 mmol) and the reaction mixture was heated at 50° C. for 2 h. After cooling to room temperature, N,N′-dimethylethylenediamine (0.08 mL, 0.67 mmol) followed by triethylamine (0.14 mL, 0.10 mmol) were added and the reaction mixture was stirred at room temperature for 3 h. The reaction mixture was then concentrated in vacuo and poured into ice cold water (80 mL). The resultant precipitate was collected by filtration and dried in vacuo. The crude material was purified by preparative HPLC (Method A) to afford 4-(6-(2-(2,6-dioxopiperidin-3-yl)-1-oxoisoindolin-5-yl)hex-5-yn-1-yl)-2-fluoro-N-(2-((R)-1-methylpyrrolidin-2-yl)-1H-pyrrolo[3,2-c]pyridin-6-yl)benzamide (Example 223, 0.06 g, 29%) as an off-white solid. ¹H NMR (400 MHz, DMSO-d₆) δ ppm 1.58-1.62 (m, 2H), 1.76-1.98 (m, 5H), 1.99-2.01 (m, 1H), 2.11-2.15 (m, 1H), 2.16 (s, 3H), 2.22-2.28 (m, 1H), 2.38-2.44 (m, 1H), 2.54-2.62 (m, 2H), 2.70-2.74 (m, 2H), 2.85-2.95 (m, 1H), 3.10-3.17 (m, 1H), 3.27-3.30 (m, 1H), 3.32-3.35 (m, 1H), 4.27-4.35 (m, 1H), 4.40-4.47 (m, 1H), 5.08-5.12 (m, 1H), 6.38 (s, 1H), 7.18-7.24 (m, 2H), 7.50 (d, J=8.75 Hz, 1H), 7.62 (s, 1H), 7.65-7.72 (m, 2H), 8.20 (s, 1H), 8.46 (s, 1H), 10.20 (d, J=4.0 Hz, 1H), 11.0 (br s, 1H), 11.40 (br s, 1H). Mass spec m/z 661.0 [M+H]⁺.

### Example 224 was Prepared Following Scheme 216

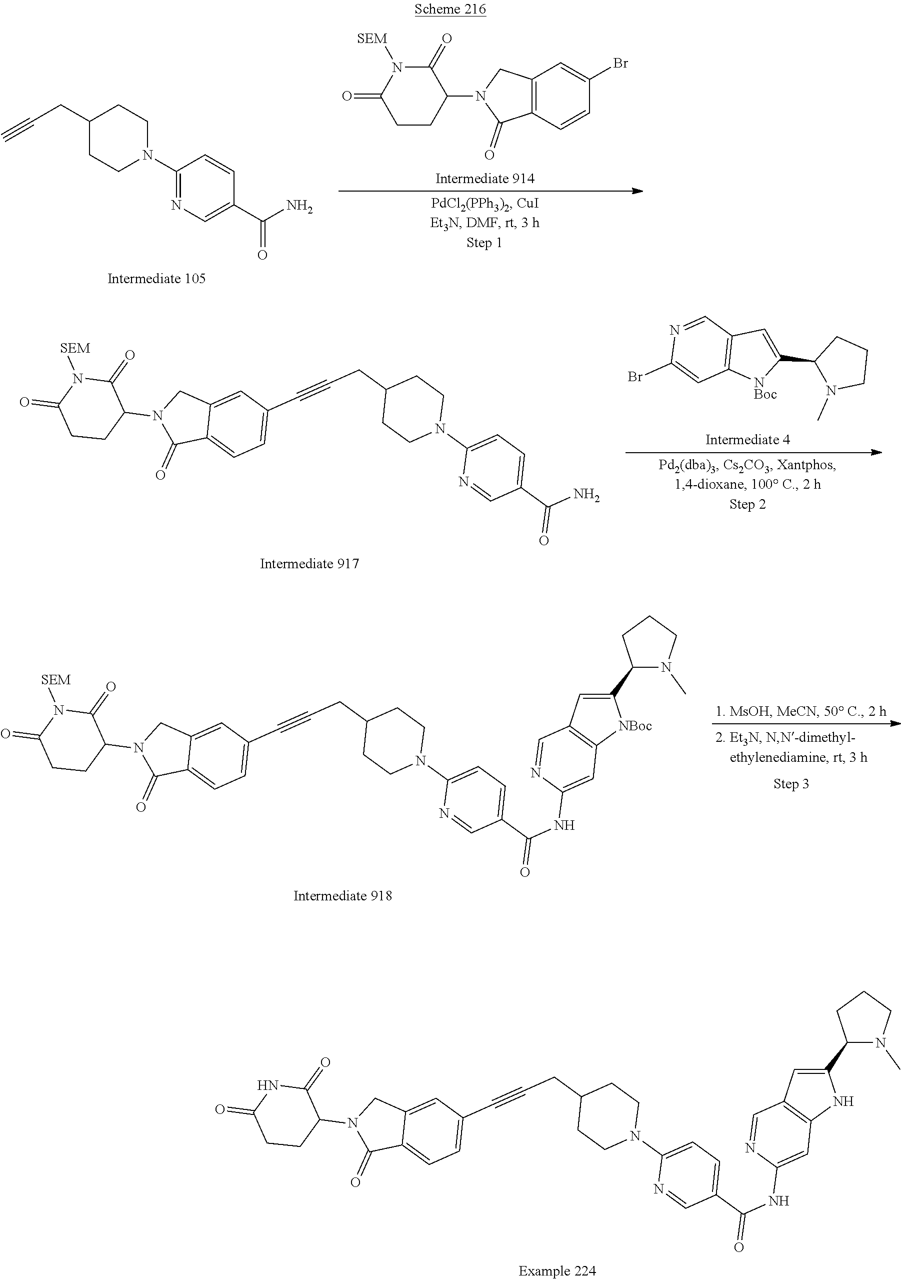 [SMILES: C#CCC1CCN(c2ccc(C(N)=O)cn2)CC1 ; C[Si](C)(C)CCOCN1C(=O)CCC(N2Cc3cc(Br)ccc3C2=O)C1=O ; C[Si](C)(C)CCOCN1C(=O)CCC(N2Cc3cc(C#CCC4CCN(c5ccc(C(N)=O)cn5)CC4)ccc3C2=O)C1=O ; CN1CCC[C@@H]1c1cc2cnc(Br)cc2n1C(=O)OC(C)(C)C ; CN1CCC[C@@H]1c1cc2cnc(NC(=O)c3ccc(N4CCC(CC#Cc5ccc6c(c5)CN(C5CCC(=O)N(COCC[Si](C)(C)C)C5=O)C6=O)CC4)nc3)cc2n1C(=O)OC(C)(C)C ; CN1CCC[C@@H]1c1cc2cnc(NC(=O)c3ccc(N4CCC(CC#Cc5ccc6c(c5)CN(C5CCC(=O)NC5=O)C6=O)CC4)nc3)cc2[nH]1]

### Step 1

### Intermediate 917: 6-(4-(3-(2-(2,6-dioxo-1-((2-(trimethylsilyl)ethoxy)methyl)piperidin-3-yl)-1-oxoisoindolin-5-yl)prop-2-yn-1-yl)piperidin-1-yl)nicotinamide

To a solution of 6-(4-(prop-2-yn-1-yl)piperidin-1-yl)nicotinamide (Intermediate 105, 0.3 g, 1.23 mmol) in dimethylformamide (5 mL) was added 3-(5-bromo-1-oxoisoindolin-2-yl)-1-((2-(trimethylsilyl)ethoxy)methyl)piperidine-2,6-dione (Intermediate 914, 0.4 g, 1.23 mmol) followed by triethylamine (0.7 mL, 4.92 mmol). The reaction mixture was purged with argon for 15 min then copper (I) iodide (0.03 g, 0.12 mmol) and PdCl₂(PPh₃)₂ (0.1 g, 0.12 mmol) were added. The reaction mixture was further purged with argon for 10 min and stirred at room temperature for 3 h. After completion, the reaction mixture was poured into water (50 mL) and extracted with ethyl acetate (3×100 mL). The combined organic layers were dried over anhydrous Na₂SO₄ and concentrated in vacuo. The crude material was purified by combi-flash chromatography, by eluting with 90% ethyl acetate in heptane, to afford 6-(4-(3-(2-(2,6-dioxo-1-((2-(trimethylsilyl)ethoxy)methyl)piperidin-3-yl)-1-oxoisoindolin-5-yl)prop-2-yn-1-yl)piperidin-1-yl) nicotinamide (Intermediate 917, 0.50 g, 60%) as a yellow solid. ¹H NMR (400 MHz, DMSO-d₆) δ ppm −0.02 (s, 9H), 0.79-0.87 (m, 2H), 1.14-1.35 (m, 4H), 1.81-1.92 (m, 3H), 2.01-2.09 (m, 1H), 2.31-2.41 (m, 2H), 2.78-2.95 (m, 2H), 3.00-3.11 (m, 1H), 3.48-3.56 (m, 2H), 4.00-4.05 (m, 1H), 4.24-4.31 (m, 1H), 4.42-4.48 (m, 2H), 5.01-5.08 (m, 2H), 5.20-5.25 (m, 1H), 6.85 (d, J=9.12 Hz, 1H), 7.09 (br s, 1H), 7.49 (d, J=7.88 Hz, 1H), 7.59 (br s, 1H), 7.66-7.75 (m, 2H), 7.93 (dd, J=9.12, 2.49 Hz, 1H), 8.57-8.62 (m, 1H). Mass spec m/z 616.0 [M+H]⁺.

### Step 2

### Intermediate 918: tert-butyl 6-(6-(4-(3-(2-(2,6-dioxo-1-((2-(trimethylsilyl)ethoxy)methyl)piperidin-3-yl)-1-oxoisoindolin-5-yl)prop-2-yn-1-yl)piperidin-1-yl)nicotinamido)-2-((R)-1-methylpyrrolidin-2-yl)-1H-pyrrolo[3,2-c]pyridine-1-carboxylate

To a solution of 6-(4-(3-(2-(2,6-dioxo-1-((2-(trimethylsilyl)ethoxy)methyl)piperidin-3-yl)-1-oxoisoindolin-5-yl)prop-2-yn-1-yl)piperidin-1-yl)nicotinamide (Intermediate 917, 0.32 g, 0.53 mmol) in 1,4-dioxane (5 mL) was added tert-butyl (R)-6-bromo-2-(1-methylpyrrolidin-2-yl)-1H-pyrrolo[3,2-c]pyridine-1-carboxylate (Intermediate 4, 0.25 g, 0.66 mmol) followed by cesium carbonate (0.65 g, 2.0 mmol). The reaction mixture was purged with argon for 15 min, then Pd₂(dba)₃ (0.09 g, 0.09 mmol) and Xantphos (0.12 g, 0.20 mmol) were added. The reaction mixture was further purged with argon for 10 min and heated at 100° C. for 2 h. After completion, the reaction mixture was concentrated in vacuo and the crude material was purified by combi-flash chromatography, by eluting with 90% ethyl acetate in heptane, to afford tert-butyl 6-(6-(4-(3-(2-(2,6-dioxo-1-((2-(trimethylsilyl)ethoxy)methyl)piperidin-3-yl)-1-oxoisoindolin-5-yl)prop-2-yn-1-yl)piperidin-1-yl)nicotinamido)-2-((R)-1-methylpyrrolidin-2-yl)-1H-pyrrolo[3,2-c]pyridine-1-carboxylate (Intermediate 918, 0.24 g, 40%) as an off-white solid which was used for the next step without further purification. Mass spec m/z 815.4 [M−100+H]⁺.

### Step 3

### Example 224: of 6-(4-(3-(2-(2,6-dioxopiperidin-3-yl)-1-oxoisoindolin-5-yl)prop-2-yn-1-yl) piperidin-1-yl)-N-(2-((R)-1-methylpyrrolidin-2-yl)-1H-pyrrolo[3,2-c]pyridin-6-yl)nicotinamide

To a solution of tert-butyl 6-(6-(4-(3-(2-(2,6-dioxo-1-((2-(trimethylsilyl)ethoxy)methyl)piperidin-3-yl)-1-oxoisoindolin-5-yl)prop-2-yn-1-yl)piperidin-1-yl)nicotinamido)-2-((R)-1-methylpyrrolidin-2-yl)-1H-pyrrolo[3,2-c]pyridine-1-carboxylate (Intermediate 918, 0.2 g, 0.21 mmol) in acetonitrile (4 mL) was added methanesulfonic acid (0.21 mL, 3.27 mmol) and the reaction mixture was heated at 50° C. for 2 h. After cooling to room temperature, N,N′-dimethylethylenediamine (0.155 mL, 1.31 mmol) followed by triethylamine (0.44 g, 4.37 mmol) were added and the reaction mixture was stirred at room temperature for 3 h. After completion, the reaction mixture was concentrated in vacuo, the resulting residue was diluted with water (100 mL), the resultant precipitate was collected by filtration and dried in vacuo. The crude material was purified by preparative HPLC (Method A) to afford 6-(4-(3-(2-(2,6-dioxopiperidin-3-yl)-1-oxoisoindolin-5-yl)prop-2-yn-1-yl)piperidin-1-yl)-N-(2-((R)-1-methylpyrrolidin-2-yl)-1H-pyrrolo[3,2-c]pyridin-6-yl)nicotinamide (Example 224, 0.02 g, 19%) as an off-white solid. ¹H NMR (400 MHz, DMSO-d₆) δ ppm 1.26-1.36 (m, 2H), 1.75-1.98 (m, 8H), 2.11-2.14 (m, 1H), 2.16 (s, 3H), 2.35-2.47 (m, 2H), 2.55-2.59 (m, 1H), 2.84-2.98 (m, 3H), 3.14 (t, J=8.01 Hz, 1H), 3.25-3.30 (m, 1H), 3.33-3.37 (m, 1H), 4.31-4.45 (m, 2H), 4.48-4.52 (m, 2H), 5.05-5.10 (m, 1H), 6.37 (s, 1H), 6.89 (d, J=9.20 Hz, 1H), 7.50 (d, J=7.80 Hz, 1H), 7.59 (s, 1H), 7.68 (d, J=7.80 Hz, 1H), 8.13-8.18 (m, 2H), 8.47 (s, 1H), 8.80 (d, J=2.45 Hz, 1H), 10.26 (br s, 1H), 10.98 (br s, 1H), 11.33 (br s, 1H). Mass spec m/z 685.1 [M+H]⁺.

### Example 225 was Prepared Following Scheme 217

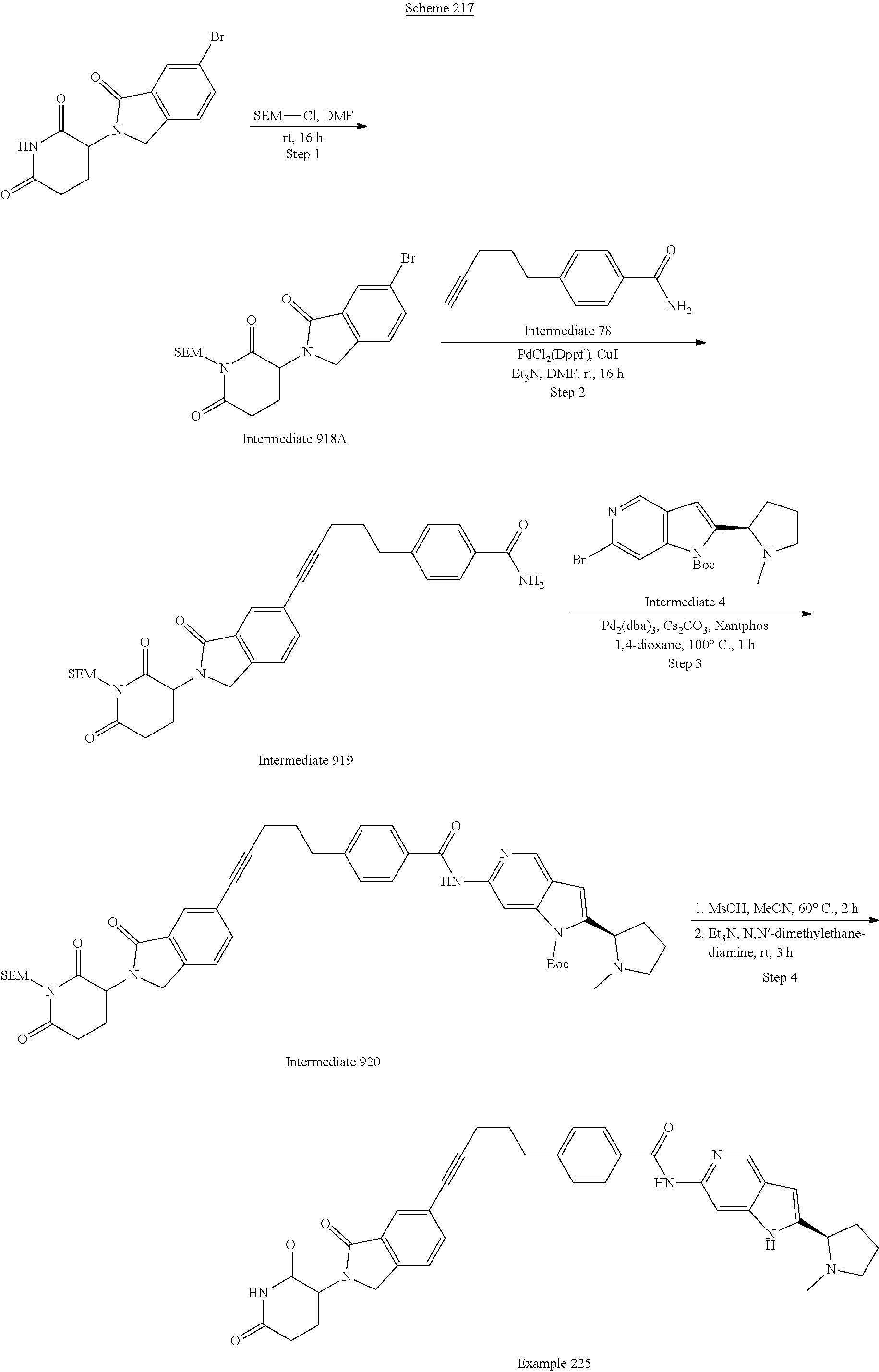

### Step 1

### Intermediate 918A: 3-(6-bromo-1-oxoisoindolin-2-yl)-1-((2-(trimethylsilyl)ethoxy)methyl)piperidine-2,6-dione

To a cooled (0° C.) solution of 3-(6-bromo-1-oxoisoindolin-2-yl)piperidine-2,6-dione (CAS No. 2304513-76-4, 5.0 g, 15.5 mmol) in dimethylformamide (50 mL) was added 1,8-diazabicyclo[5.4.0]undec-7-ene (8.33 mL, 54.2 mmol) and the mixture was stirred for 10 min. 2-(Trimethylsilyl)ethoxymethyl chloride (7.1 mL, 38.7 mmol) was then added and the reaction mixture was stirred at room temperature for 16 h. The reaction mixture was quenched with water (700 mL) and extracted with ethyl acetate (2×250 mL). The combined organic layers were dried over anhydrous Na₂SO₄ and concentrated in vacuo. The crude material was purified by combi-flash chromatography, by eluting with 50% ethyl acetate in heptane, to afford 3-(6-bromo-1-oxoisoindolin-2-yl)-1-((2-(trimethylsilyl)ethoxy)methyl)piperidine-2,6-dione (Intermediate 918A, 3.0 g, 22%) as an off-white solid. ¹H NMR (400 MHz, DMSO-d₆) δ ppm −0.03 (s, 9H), 0.81-0.86 (m, 2H), 2.01-2.10 (m, 1H), 2.30-2.41 (m, 1H), 2.75-2.82 (m, 1H), 2.99-3.11 (m, 1H), 3.46-3.57 (m, 2H), 4.26-4.31 (m, 1H), 4.45-4.50 (m, 1H), 5.00-5.07 (m, 2H), 5.20-5.25 (m, 1H), 7.60 (d, J=8.22 Hz, 1H), 7.83 (d, J=8.22 Hz, 1H), 7.87 (s, 1H). Mass spec m/z 451.0 [M−H]⁻.

### Step 2

### Intermediate 919: 4-(5-(2-(2,6-dioxo-1-((2-(trimethylsilyl)ethoxy)methyl)piperidin-3-yl)-3-oxoisoindolin-5-yl)pent-4-yn-1-yl)benzamide

To a solution of 4-(pent-4-yn-1-yl)benzamide (Intermediate 78, 0.35 g, 1.9 mmol) in dimethylformamide (15 mL) was added 3-(6-bromo-1-oxoisoindolin-2-yl)-1-((2-(trimethylsilyl)ethoxy)methyl)piperidine-2,6-dione (Intermediate 918A, 1.4 g, 1.6 mmol) followed by triethylamine (1.5 mL, 11.0 mmol). The reaction mixture was purged with argon for 15 min, then copper (I) iodide (0.03 g, 0.16 mmol) and PdCl₂(dppf) (0.12 g, 0.16 mmol) were added. The reaction mixture was further purged with argon for 10 min and stirred at room temperature for 16 h. The reaction mixture was poured into water (700 mL) and extracted with ethyl acetate (2×250 mL). The combined organic layers were washed with water (500 mL), dried over anhydrous Na₂SO₄ and concentrated in vacuo. The crude material was purified by combi-flash chromatography, by eluting with 50% ethyl acetate in heptane, to afford 4-(5-(2-(2,6-dioxo-1-((2-(trimethylsilyl)ethoxy)methyl)piperidin-3-yl)-3-oxoisoindolin-5-yl)pent-4-yn-1-yl)benzamide (Intermediate 919, 0.75 g, 86%) as an off-white solid. ¹H NMR (400 MHz, DMSO-d₆) δ ppm −0.05 (s, 9H), 0.81 (t, J=7.26 Hz, 2H), 1.81-1.91 (m, 2H), 1.95-2.04 (m, 1H), 2.30-2.41 (m, 2H), 2.72-2.81 (m, 3H), 2.96-3.09 (m, 2H), 3.46-3.53 (m, 2H), 4.26-4.31 (m, 1H), 4.45-4.50 (m, 1H), 4.98-5.05 (m, 2H), 5.17-5.22 (m, 1H), 7.23 (br s, 1H), 7.30 (d, J=7.88 Hz, 2H), 7.56-7.66 (m, 3H), 7.78 (d, J=7.88 Hz, 2H), 7.86 (br s, 1H). Mass spec m/z 559.0 [M]⁺.

### Step 3

### Intermediate 920: tert-butyl-6-(4-(5-(2-(2,6-dioxo-1-((2-(trimethylsilyl) ethoxy)methyl) piperidin-3-yl)-3-oxoisoindolin-5-yl)pent-4-yn-1-yl)benzamido)-2-((R)-1-methylpyrrolidin-2-yl)-1H-pyrrolo[3,2-c]pyridine-1-carboxylate

To a solution of 4-(5-(2-(2,6-dioxo-1-((2-(trimethylsilyl)ethoxy)methyl)piperidin-3-yl)-3-oxoisoindolin-5-yl) pent-4-yn-1-yl)benzamide (Intermediate 919, 0.70 g, 1.25 mmol) in 1,4-dioxane (20 mL) was added tert-butyl-(R)-6-bromo-2-(1-methylpyrrolidin-2-yl)-1H-pyrrolo[3,2-c]pyridine-1-carboxylate (Intermediate 4, 0.52 g, 1.37 mmol) followed by cesium carbonate (1.28 g, 3.75 mmol). The reaction mixture was purged with argon for 15 min and then Pd₂(dba)₃ (0.15 g, 0.18 mmol) followed by Xantphos (0.22 g, 0.37 mmol) were added. The reaction mixture was further purged with argon for 10 min, then heated at 100° C. for 1 h. The reaction mixture was concentrated in vacuo and the crude material was purified by combi-flash chromatography, by eluting with 5% MeOH in DCM, to afford tert-butyl-6-(4-(5-(2-(2,6-dioxo-1-((2-(trimethylsilyl)ethoxy)methyl)piperidin-3-yl)-3-oxoisoindolin-5-yl)pent-4-yn-1-yl)benzamido)-2-((R)-1-methylpyrrolidin-2-yl)-1H-pyrrolo[3,2-c]pyridine-1-carboxylate (Intermediate 920, 0.48 g, 27%) as a yellow solid. ¹H NMR (400 MHz, DMSO-d₆) δ ppm −0.03 (s, 9H), 0.80-0.89 (m, 2H), 1.60-1.65 (m, 2H), 1.69 (s, 9H), 1.70-1.75 (m, 3H), 1.90-1.97 (m, 2H), 2.35 (s, 3H), 2.36-2.41 (m, 2H), 2.51-2.57 (m, 3H), 2.75-2.89 (m, 3H), 3.09-3.16 (m, 2H), 3.39-3.40 (m, 1H), 3.46-3.59 (m, 2H), 3.88-3.94 (m, 1H), 4.29-4.34 (m, 1H), 4.48-4.53 (m, 1H), 5.01-5.10 (m, 1H), 6.75 (s, 1H), 7.29-7.40 (m, 3H), 7.58-7.73 (m, 2H), 8.00 (d, J=7.88 Hz, 2H), 8.59 (s, 1H), 8.93 (s, 1H), 10.62 (br s, 1H). Mass spec m/z 859.4 [M+H]⁺.

### Step 4

### Example 225: 4-(5-(2-(2,6-dioxopiperidin-3-yl)-3-oxoisoindolin-5-yl)pent-4-yn-1-yl)-N-(2-((R)-1-methylpyrrolidin-2-yl)-1H-pyrrolo[3,2-c]pyridin-6-yl)benzamide

To a solution of tert-butyl-6-(4-(5-(2-(2,6-dioxo-1-((2-(trimethylsilyl)ethoxy)methyl)piperidin-3-yl)-3-oxoisoindolin-5-yl)pent-4-yn-1-yl)benzamido)-2-((R)-1-methylpyrrolidin-2-yl)-1H-pyrrolo[3,2-c]pyridine-1-carboxylate (Intermediate 920, 0.47 g, 0.54 mmol) in acetonitrile (7 mL) was added methanesulfonic acid (0.36 mL, 5.48 mmol) and the reaction mixture was heated at 60° C. for 2 h. The reaction mixture was cooled to room temperature, then N,N′-dimethylethylenediamine (0.32 mL, 2.72 mmol) followed by triethylamine (1.53 mL, 10.92 mmol) were added and the mixture was stirred at room temperature for 3 h. The reaction mixture was concentrated in vacuo and the resultant residue was diluted with water (100 mL). The resultant precipitate was collected by filtration and dried in vacuo. The crude solid was purified by preparative HPLC (Method A) to afford 4-(5-(2-(2,6-dioxopiperidin-3-yl)-3-oxoisoindolin-5-yl)pent-4-yn-1-yl)-N-(2-((R)-1-methylpyrrolidin-2-yl)-1H-pyrrolo[3,2-c]pyridin-6-yl)benzamide (Example 225, 0.06 g, 19%) as an off-white solid.

### Example 226 as Prepared Following Scheme 218

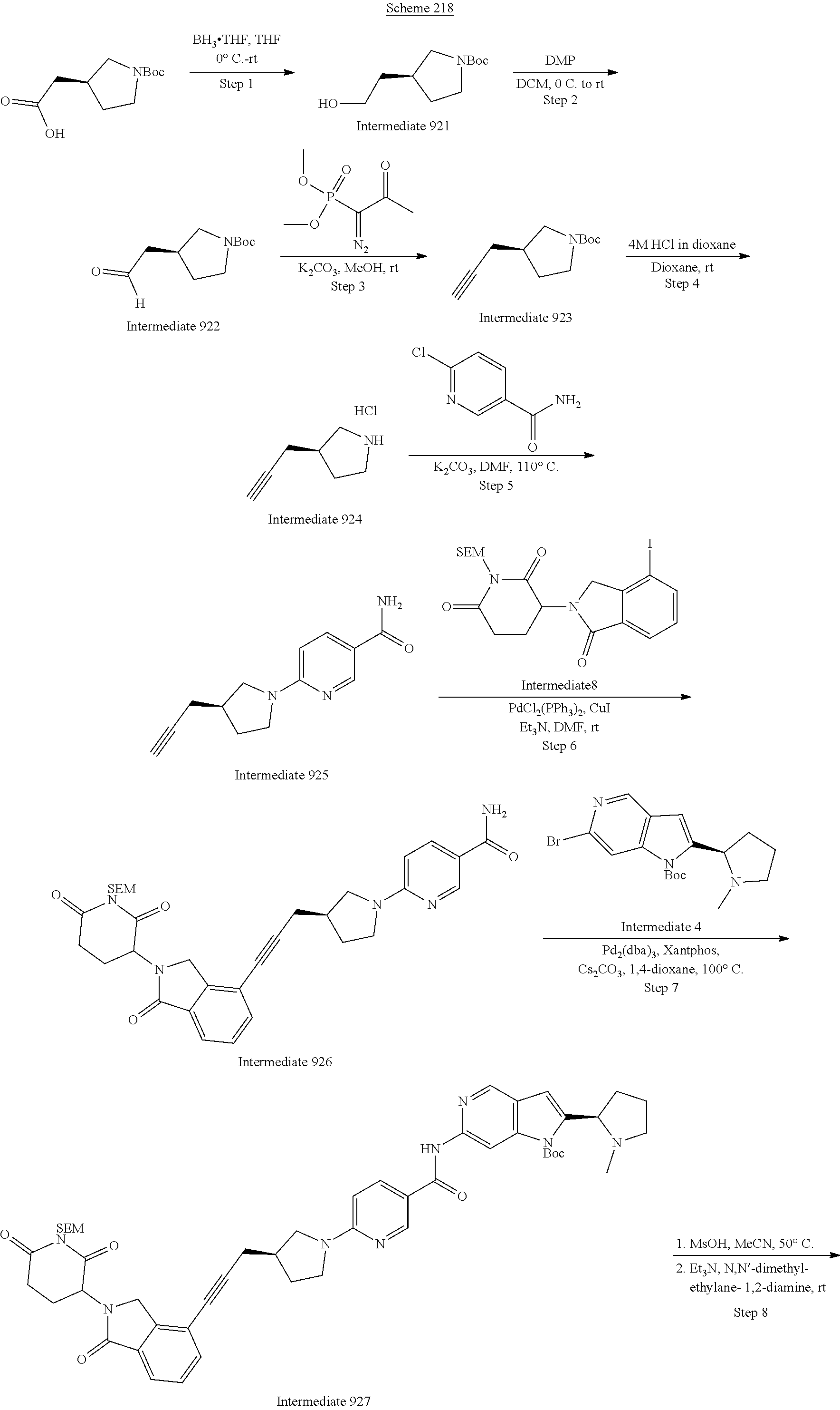

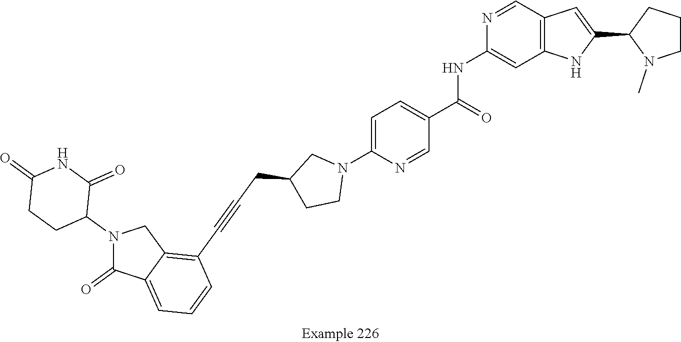 [SMILES: CN1CCC[C@@H]1c1cc2cnc(NC(=O)c3ccc(N4CC[C@@H](CC#Cc5cccc6c5CN(C5CCC(=O)NC5=O)C6=O)C4)nc3)cc2[nH]1]

### Step 1

### Intermediate 921: tert-butyl-(S)-3-(2-hydroxyethyl)pyrrolidine-1-carboxylate

To a cooled (0° C.) solution of (S)-2-(1-(tert-butoxycarbonyl)pyrrolidin-3-yl)acetic acid (CAS No. 204688-61-9, 5.0 g, 22.0 mmol) in tetrahydrofuran (50 mL) was added 1M borane tetrahydrofuran complex solution in tetrahydrofuran (65 mL, 65.0 mmol) and the reaction mixture was then stirred at room temperature for 16 h. After completion the reaction mixture was cooled to 0° C., quenched with methanol (100 mL), then concentrated in vacuo. The crude material was purified by combi-flash chromatography, by eluting with 60% ethyl acetate in heptane, to afford tert-butyl-(S)-3-(2-hydroxyethyl)pyrrolidine-1-carboxylate (Intermediate 921, 4.1 g, 87%) as a yellow liquid. ¹H NMR (400 MHz, DMSO-d₆) 1.38 (s, 9H), 1.50-1.59 (m, 3H), 1.86-1.98 (m, 1H), 2.11-2.19 (m, 1H), 2.74-2.79 (m, 1H), 3.02-3.13 (m, 1H), 3.25-3.35 (m, 1H), 3.35-3.47 (m, 2H), 3.70-3.81 (m, 1H). Mass spec m/z 160.1 [M−56-H]⁻.

### Step 2

### Intermediate 922: tert-butyl-(S)-3-(2-oxoethyl)pyrrolidine-1-carboxylate

To a cooled (0° C.) solution of tert-butyl-(S)-3-(2-hydroxyethyl)pyrrolidine-1-carboxylate (Intermediate 921, 4.1 g, 19.0 mmol) in dichloromethane (50 mL) was added Dess-Martin periodinane (12.0 g, 29.0 mmol) and the reaction mixture was stirred at room temperature for 1 h. The reaction mixture was diluted with saturated aqueous sodium bicarbonate solution (200 mL) and extracted with dichloromethane (2×100 mL). The combined organic layers were dried over anhydrous Na₂SO₄, filtered and concentrated in vacuo to afford tert-butyl-(S)-3-(2-oxoethyl)pyrrolidine-1-carboxylate (Intermediate 922, 3.7 g) as a colourless liquid, which was used for the next step without further purification.

### Step 3

### Intermediate 923: tert-butyl-(R)-3-(prop-2-yn-1-yl)pyrrolidine-1-carboxylate

To a solution of tert-butyl-(S)-3-(2-oxoethyl)pyrrolidine-1-carboxylate (Intermediate 922, 3.7 g, 15.0 mmol) in methanol (40 mL) was added potassium carbonate (4.4 g, 30.0 mmol) followed by dimethyl(1-diazo-2-oxopropyl)phosphonate (CAS No. 90965-06-3, 3.7 g, 19.0 mmol). The reaction mixture was stirred at room temperature for 3 h then concentrated in vacuo. The crude material was purified by combi-flash chromatography, by eluting with 40% ethyl acetate in heptane, to afford tert-butyl-(R)-3-(prop-2-yn-1-yl)pyrrolidine-1-carboxylate (Intermediate 923, 1.8 g, 49%) as a gummy liquid. ¹H NMR (400 MHz, DMSO-d₆) δ ppm 1.39 (s, 9H), 1.50-1.65 (m, 1H), 1.81-1.95 (m, 1H), 2.18-2.26 (m, 3H), 2.83 (s, 1H), 2.87-2.96 (m, 2H), 3.11-3.24 (m, 1H), 3.34-3.44 (m, 1H).

### Step 4

### Intermediate 924: (R)-3-(prop-2-yn-1-yl)pyrrolidine hydrochloride

To a cooled (0° C.) solution of tert-butyl-(R)-3-(prop-2-yn-1-yl)pyrrolidine-1-carboxylate (Intermediate 923, 1.5 g, 7.2 mmol) in 1,4-dioxane (30 mL) was added 4M hydrochloric acid in 1,4-dioxane (20 mL, 80.0 mmol) and the reaction mixture was stirred at room temperature for 16 h. The reaction mixture was concentrated in vacuo to afford (R)-3-(prop-2-yn-1-yl)pyrrolidine hydrochloride (Intermediate 924, 0.8 g) as a colourless liquid, which was used for the next step without further purification. ¹H NMR (400 MHz, DMSO-d₆) δ ppm 1.56-1.68 (m, 1H), 1.96-2.09 (m, 1H), 2.36-2.44 (m, 2H), 2.73-2.84 (m, 1H), 2.91 (br s, 1H), 3.04-3.15 (m, 1H), 3.15-3.30 (m, 3H), 3.37-3.51 (m, 1H), 9.43 (br s, 1H). Mass spec m/z 110.1 [M+H]⁺.

### Step 5

### Intermediate 925: (R)-6-(3-(prop-2-yn-1-yl)pyrrolidin-1-yl)nicotinamide

To a solution of (R)-3-(prop-2-yn-1-yl)pyrrolidine hydrochloride (Intermediate 924, 0.8 g, 6.59 mmol) and 6-chloronicotinamide (CAS No. 6271-78-9, 1.23 g, 7.91 mmol) in dimethylformamide (10 mL) was added potassium carbonate (2.73 g, 19.8 mmol). The reaction mixture was stirred at 110° C. for 16 h then diluted with water (100 mL) and extracted with ethyl acetate (3×100 mL). The combined organic layers were dried over anhydrous Na₂SO₄ and concentrated in vacuo. The crude material was purified by combi-flash chromatography, by eluting with 45% ethyl acetate in heptane, to afford (R)-6-(3-(prop-2-yn-1-yl)pyrrolidin-1-yl)nicotinamide (Intermediate 925, 1.0 g, 66%) as an off-white solid. ¹H NMR (400 MHz, DMSO-d₆) δ ppm 1.71-1.81 (m, 1H), 2.08-2.16 (m, 1H), 2.35 (d, J=6.0 Hz, 2H), 2.43-2.48 (m, 1H), 2.86 (s, 1H), 3.16 (t, J=7.60 Hz, 1H), 3.34-3.43 (m, 1H), 3.51-3.59 (m, 1H), 3.64 (t, J=7.60 Hz, 1H), 6.42 (d, J=8.80 Hz, 1H), 7.05 (br s, 1H), 7.69 (br s, 1H), 7.92 (d, J=8.80 Hz, 1H), 8.58 (s, 1H). Mass spec m/z 230.4 [M+H]⁺.

### Step 6

### Intermediate 926: 6-((3R)-3-(3-(2-(2,6-dioxo-1-((2-(trimethylsilyl)ethoxy)methyl)piperidin-3-yl)-1-oxoisoindolin-4-yl)prop-2-yn-1-yl)pyrrolidin-1-yl)nicotinamide

To a solution of (R)-6-(3-(prop-2-yn-1-yl)pyrrolidin-1-yl)nicotinamide (Intermediate 925, 0.30 g, 1.30 mmol) in dimethylformamide (2 mL) was added 3-(4-iodo-1-oxoisoindolin-2-yl)-1-((2-(trimethylsilyl)ethoxy)methyl) piperidine-2,6-dione (Intermediate 8, 0.65 g, 1.30 mmol) followed by triethylamine (6.41 mL, 45.79 mmol). The reaction mixture was purged with argon for 15 min, then copper (I) iodide (0.02 g, 0.13 mmol) and PdCl₂(PPh₃)₂ (0.09 g, 0.13 mmol) were added. The reaction mixture was further purged with argon for 10 min and stirred at room temperature for 2 h. The reaction mixture was diluted with water (50 mL) and extracted with ethyl acetate (2×100 mL). The combined organic layers were dried over anhydrous Na₂SO₄ and concentrated in vacuo. The crude material was purified by combi-flash chromatography, by eluting with 80% ethyl acetate in heptane, to afford 6-((3R)-3-(3-(2-(2,6-dioxo-1-((2-(trimethylsilyl)ethoxy)methyl)piperidin-3-yl)-1-oxoisoindolin-4-yl)prop-2-yn-1-yl)pyrrolidin-1-yl)nicotinamide (Intermediate 926, 0.4 g, 51%) as a yellow solid. ¹H NMR (400 MHz, DMSO-d₆) δ ppm −0.03 (s, 9H), 0.77-0.88 (m, 2H), 1.82-1.93 (m, 1H), 2.00-2.05 (m, 1H), 2.19-2.23 (m, 1H), 2.31-2.41 (m, 1H), 2.56-2.65 (m, 1H), 2.65-2.71 (m, 2H), 2.72-2.75 (m, 1H), 3.00-3.13 (m, 1H), 3.24-3.30 (m, 1H), 3.38-3.48 (m, 1H), 3.48-3.56 (m, 2H), 3.56-3.65 (m, 1H), 3.67-3.75 (m, 1H), 4.25-4.29 (m, 1H), 4.45-4.49 (m, 1H), 4.99-5.10 (m, 2H), 5.22-5.28 (m, 1H), 6.45 (d, J=8.0 Hz, 1H), 7.05 (br s, 1H), 7.49-7.57 (m, 1H), 7.63-7.76 (m, 3H), 7.88-7.98 (m, 1H), 8.58 (s, 1H). Mass spec m/z 602.2 [M+H]⁺.

### Step 7

### Intermediate 927: tert-butyl 6-(6-((3R)-3-(3-(2-(2,6-dioxo-1-((2-(trimethylsilyl)ethoxy)methyl)piperidin-3-yl)-1-oxoisoindolin-4-yl)prop-2-yn-1-yl)pyrrolidin-1-yl)nicotinamido)-2-((R)-1-methylpyrrolidin-2-yl)-1H-pyrrolo[3,2-c]pyridine-1-carboxylate

To a solution of 6-((3R)-3-(3-(2-(2,6-dioxo-1-((2-(trimethylsilyl)ethoxy)methyl)piperidin-3-yl)-1-oxoisoindolin-4-yl)prop-2-yn-1-yl)pyrrolidin-1-yl)nicotinamide (Intermediate 926, 0.37 g, 0.61 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 4, 0.30 g, 0.78 mmol) followed by cesium carbonate (0.45 g, 2.36 mmol). The reaction mixture was purged with argon for 15 min, then Pd₂(dba)₃ (0.11 g, 0.11 mmol) and Xantphos (0.14 g, 0.23 mmol) were added. The reaction mixture was further purged with argon for 10 min, then stirred at 100° C. for 3 h. The reaction mixture was concentrated in vacuo and the crude material was purified by combi-flash chromatography, by eluting with 10% MeOH in DCM, to afford tert-butyl 6-(6-((3R)-3-(3-(2-(2,6-dioxo-1-((2-(trimethylsilyl)ethoxy)methyl)piperidin-3-yl)-1-oxoisoindolin-4-yl)prop-2-yn-1-yl)pyrrolidin-1-yl)nicotinamido)-2-((R)-1-methylpyrrolidin-2-yl)-1H-pyrrolo[3,2-c]pyridine-1-carboxylate (Intermediate 927, 0.3 g, 42%) as a yellow solid. ¹H NMR (400 MHz, DMSO-d₆) δ ppm −0.05 (s, 9H), 0.76-0.87 (m, 2H), 1.63 (s, 9H), 1.65-1.76 (m, 4H), 2.00-2.12 (m, 1H), 2.16-2.28 (m, 3H), 2.35 (s, 3H), 2.39-2.43 (m, 2H), 2.60-2.77 (m, 4H), 3.02-3.17 (m, 2H), 3.43-3.57 (m, 3H), 3.60-3.70 (m, 1H), 3.69-3.79 (m, 1H), 3.87-3.94 (m, 1H), 4.23-4.33 (m, 1H), 4.43-4.53 (m, 1H), 4.98-5.09 (m, 2H), 5.23-5.28 (m, 1H), 6.51 (d, J=8.80 Hz, 1H), 6.74 (s, 1H), 7.50-7.58 (m, 1H), 7.69 (d, J=7.60 Hz, 1H), 7.74 (d, J=7.60 Hz, 1H), 8.08-8.18 (m, 1H), 8.57 (s, 1H), 8.78-8.80 (m, 1H), 8.92 (s, 1H), 10.46 (s, 1H). Mass spec m/z 900.7 [M+H]⁺.

### Step 8

### Example 226: 6-((3R)-3-(3-(2-(2,6-dioxopiperidin-3-yl)-1-oxoisoindolin-4-yl)prop-2-yn-1-yl)pyrrolidin-1-yl)-N-(2-((R)-1-methylpyrrolidin-2-yl)-1H-pyrrolo[3,2-c]pyridin-6-yl)nicotinamide

To a solution of tert-butyl 6-(6-((3R)-3-(3-(2-(2,6-dioxo-1-((2-(trimethylsilyl)ethoxy)methyl)piperidin-3-yl)-1-oxoisoindolin-4-yl)prop-2-yn-1-yl)pyrrolidin-1-yl)nicotinamido)-2-((R)-1-methylpyrrolidin-2-yl)-1H-pyrrolo[3,2-c]pyridine-1-carboxylate (Intermediate 927, 0.30 g, 0.33 mmol) in acetonitrile (5 mL) was added methanesulfonicacid (0.33 mL, 4.99 mmol) and the reaction mixture was stirred at 50° C. for 2 h. The reaction mixture was cooled to room temperature, then N,N′-dimethylethylenediamine (0.31 mL, 2.66 mmol) followed by triethylamine (0.93 mL, 6.65 mmol) were added and the mixture was stirred at room temperature for 2 h. The reaction mixture was poured into ice cold water (80 mL) and the resultant precipitate was collected by filtration and dried in vacuo. The crude material was purified by preparative HPLC (Method A) to afford 6-((3R)-3-(3-(2-(2,6-dioxopiperidin-3-yl)-1-oxoisoindolin-4-yl)prop-2-yn-1-yl)pyrrolidin-1-yl)-N-(2-((R)-1-methylpyrrolidin-2-yl)-1H-pyrrolo[3,2-c]pyridin-6-yl)nicotinamide (Example 226, 0.11 g, 53%) as an off-white solid. ¹H NMR (400 MHz, DMSO-d₆) δ ppm 1.85-2.00 (m, 5H), 2.07-2.28 (m, 5H), 2.32-2.39 (m, 3H), 2.52-2.57 (m, 1H), 2.59-2.69 (m, 1H), 2.70 (s, 3H), 2.84-2.97 (m, 1H), 3.12-3.21 (m, 1H), 3.42-3.52 (m, 1H), 3.62-3.68 (m, 1H), 3.71-3.80 (m, 1H), 4.28-4.38 (m, 1H), 4.42-4.51 (m, 1H), 5.10-5.16 (m, 1H), 6.35-6.53 (m, 2H), 7.48-7.57 (m, 1H), 7.59-7.77 (m, 2H), 8.08-8.21 (m, 2H), 8.49 (s, 1H), 8.78-8.80 (m, 1H), 10.21 (br s, 1H), 10.99 (br s, 1H), 11.35 (br s, 1H). Mass spec m/z 671.2 [M+H]⁺.

### Example 227 was Synthesised Following Scheme 219

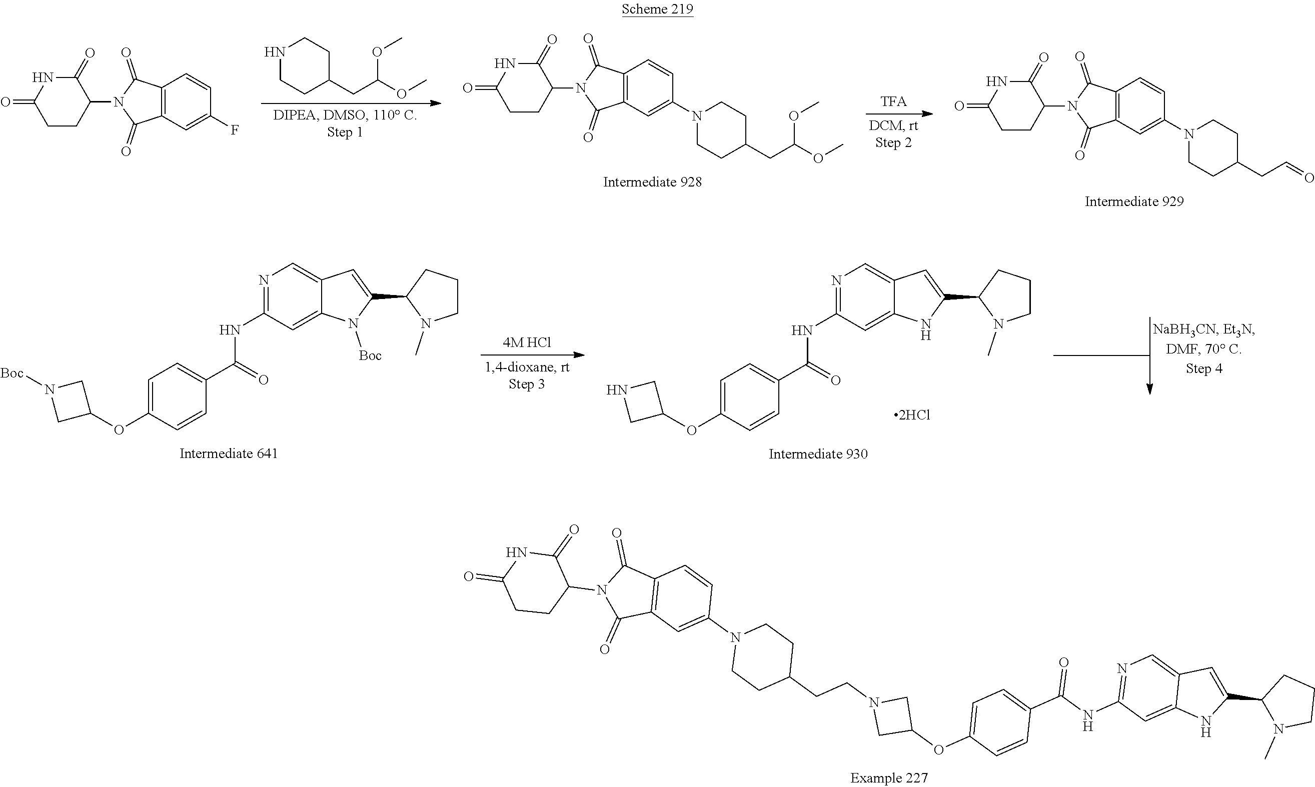

### Step 1

### Intermediate 928: 5-(4-(2,2-dimethoxyethyl)piperidin-1-yl)-2-(2,6-dioxopiperidin-3-yl)isoindoline-1,3-dione

To a solution of 4-(2,2-dimethoxyethyl)piperidine (CAS No. 15871-71-3, 2.8 g, 16.2 mmol) in dimethyl sulfoxide (50 mL) was added N,N-diisopropylethylamine (11.3 mL, 64.6 mmol) and the reaction mixture was stirred at room temperature for 5 min. 2-(2,6-dioxopiperidin-3-yl)-5-fluoroisoindoline-1,3-dione (CAS No. 835616-61-0, 4.46 g, 16.2 mmol) was then added and the reaction mixture was heated at 110° C. for 16 h. The reaction mixture was quenched with ice cold water (100 mL) and extracted with ethyl acetate (3×100 mL). The combined organic layers were dried over Na₂SO₄, filtered and concentrated in vacuo. The crude material was purified by combi-flash chromatography, by eluting with 9% MeOH in DCM, to afford 5-(4-(2,2-dimethoxyethyl)piperidin-1-yl)-2-(2,6-dioxopiperidin-3-yl)isoindoline-1,3-dione (Intermediate 928, 3.2 g, 46%) as a white solid. ¹H NMR (400 MHz, DMSO-d₆) δ ppm 1.11-1.25 (m, 2H), 1.43-1.46 (m, 2H), 1.61-1.76 (m, 4H), 1.96-2.01 (m, 1H), 2.53-2.58 (m, 2H), 2.82-2.96 (m, 3H), 3.19-3.22 (m, 5H), 3.97-4.03 (m, 2H), 4.44-4.46 (m, 1H), 5.02-5.06 (m, 1H), 7.20 (d, J=7.60 Hz, 1H), 7.28 (s, 1H), 7.62 (d, J=8.80 Hz, 1H), 11.05 (s, 1H). Mass spec m/z 430.1 [M+H]⁺.

### Step 2

### Intermediate 929: 2-(1-(2-(2,6-dioxopiperidin-3-yl)-1,3-dioxoisoindolin-5-yl)piperidin-4-yl)acetaldehyde

To a cooled (0° C.) solution of 5-(4-(2,2-dimethoxyethyl)piperidin-1-yl)-2-(2,6-dioxopiperidin-3-yl)isoindoline-1,3-dione (Intermediate 928, 0.60 g, 1.4 mmol) in dichloromethane (20 mL) was added trifluoroacetic acid (2.14 mL, 27.94 mmol) and the reaction mixture was stirred at room temperature for 4 h. The reaction mixture was concentrated in vacuo to afford as 2-(1-(2-(2,6-dioxopiperidin-3-yl)-1,3-dioxoisoindolin-5-yl)piperidin-4-yl)acetaldehyde (Intermediate 929, 0.70 g) as a yellow gummy solid, which was used for the next step without further purification. ¹H NMR (400 MHz, DMSO-d₆) δ ppm 1.19-1.29 (m, 2H), 1.71-1.77 (m, 2H), 1.98-2.19 (m, 2H), 2.39-2.43 (m, 2H), 2.52-2.62 (m, 2H), 2.85-3.05 (m, 3H), 4.01-4.05 (m, 2H), 4.87-5.87 (m, 1H), 7.21 (dd, J=8.80 Hz, 1H), 7.30-7.32 (m, 1H), 7.64 (d, J=8.80 Hz, 1H), 9.70 (s, 1H), 11.05 (s, 1H). Mass spec m/z 382.2 [M+H]⁺.

### Step 3

### Intermediate 930: (R)-4-(azetidin-3-yloxy)-N-(2-(1-methylpyrrolidin-2-yl)-1H-pyrrolo[3,2-c]pyridin-6-yl)benzamide dihydrochloride

To a solution of tert-butyl (R)-6-(4-((1-(tert-butoxycarbonyl)azetidin-3-yl)oxy)benzamido)-2-(1-methylpyrrolidin-2-yl)-1H-pyrrolo[3,2-c]pyridine-1-carboxylate (Intermediate 641, 0.5 g, 0.84 mmol) in 1,4-dioxane (30 ml) was added 4M HCl in 1,4-dioxane (15.0 mL) and the reaction mixture was stirred at room temperature for 2 h. The reaction mixture was concentrated in vacuo and the crude material was purified by combi-flash chromatography, by eluting with 12% MeOH in DCM, to afford (R)-4-(azetidin-3-yloxy)-N-(2-(1-methylpyrrolidin-2-yl)-1H-pyrrolo[3,2-c]pyridin-6-yl)benzamide dihydrochloride (Intermediate 930, 0.4 g) as a yellow solid which was used for the next step without further purification. Mass spec m/z 392.1 [M+H]⁺.

### Step 4

### Example 227: 4-((1-(2-(1-(2-(2,6-dioxopiperidin-3-yl)-1,3-dioxoisoindolin-5-yl)piperidin-4-yl)ethyl)azetidin-3-yl)oxy)-N-(2-((R)-1-methylpyrrolidin-2-yl)-1H-pyrrolo[3,2-c]pyridin-6-yl)benzamide

To a solution of 2-(1-(2-(2,6-dioxopiperidin-3-yl)-1,3-dioxoisoindolin-5-yl)piperidin-4-yl)acetaldehyde (Intermediate 929, 0.36 g, 0.94 mmol) in dimethylformamide (10.0 ml) was added (R)-4-(azetidin-3-yloxy)-N-(2-(1-methylpyrrolidin-2-yl)-1H-pyrrolo[3,2-c]pyridin-6-yl)benzamide dihydrochloride (Intermediate 930, 0.37 g, 0.94 mmol) followed by triethylamine (0.39 mL, 0.83 mmol). The reaction mixture was heated at 60° C. for 3 h. Sodium cyanoborohydride (0.35 g, 3.78 mmol) was then added and the reaction mixture was stirred at room temperature for 16 h. The reaction mixture was poured into water (10 mL), the resultant precipitate was collected by filtration and dried in vacuo. The crude material was purified by preparative HPLC (Method A) to afford 4-((1-(2-(1-(2-(2,6-dioxopiperidin-3-yl)-1,3-dioxoisoindolin-5-yl)piperidin-4-yl)ethyl)azetidin-3-yl)oxy)-N-(2-((R)-1-methylpyrrolidin-2-yl)-1H-pyrrolo[3,2-c]pyridin-6-yl)benzamide (Example 227, 0.030 g, 4%) as an off-white solid. ¹H NMR (400 MHz, DMSO-d₆) δ ppm 1.11-1.29 (m, 4H), 1.56-1.69 (m, 1H), 1.71-1.95 (m, 5H), 1.97-2.05 (m, 1H), 2.11-2.19 (m, 4H), 2.22-2.29 (m, 1H), 2.54-2.61 (m, 3H), 2.38-2.97 (m, 6H), 3.13-3.18 (m, 1H), 3.28-3.31 (m, 1H), 3.37-3.77 (m, 2H), 4.02-4.05 (m, 2H), 4.86-4.89 (m, 1H), 5.04-5.08 (m, 1H), 6.38 (s, 1H), 6.92 (d, J=8.8 Hz, 2H), 7.21-7.24 (m, 1H), 7.30 (s, 1H), 7.64 (d, J=9.20 Hz, 1H), 8.04 (d, J=8.80 Hz, 2H), 8.16 (s, 1H), 8.49 (s, 1H), 10.32 (s, 1H), 11.08 (bs, 1H), 11.35 (s, 1H). Mass spec m/z 759.2 [M+H]⁺.

### Example 228 was Synthesised Following Scheme 220

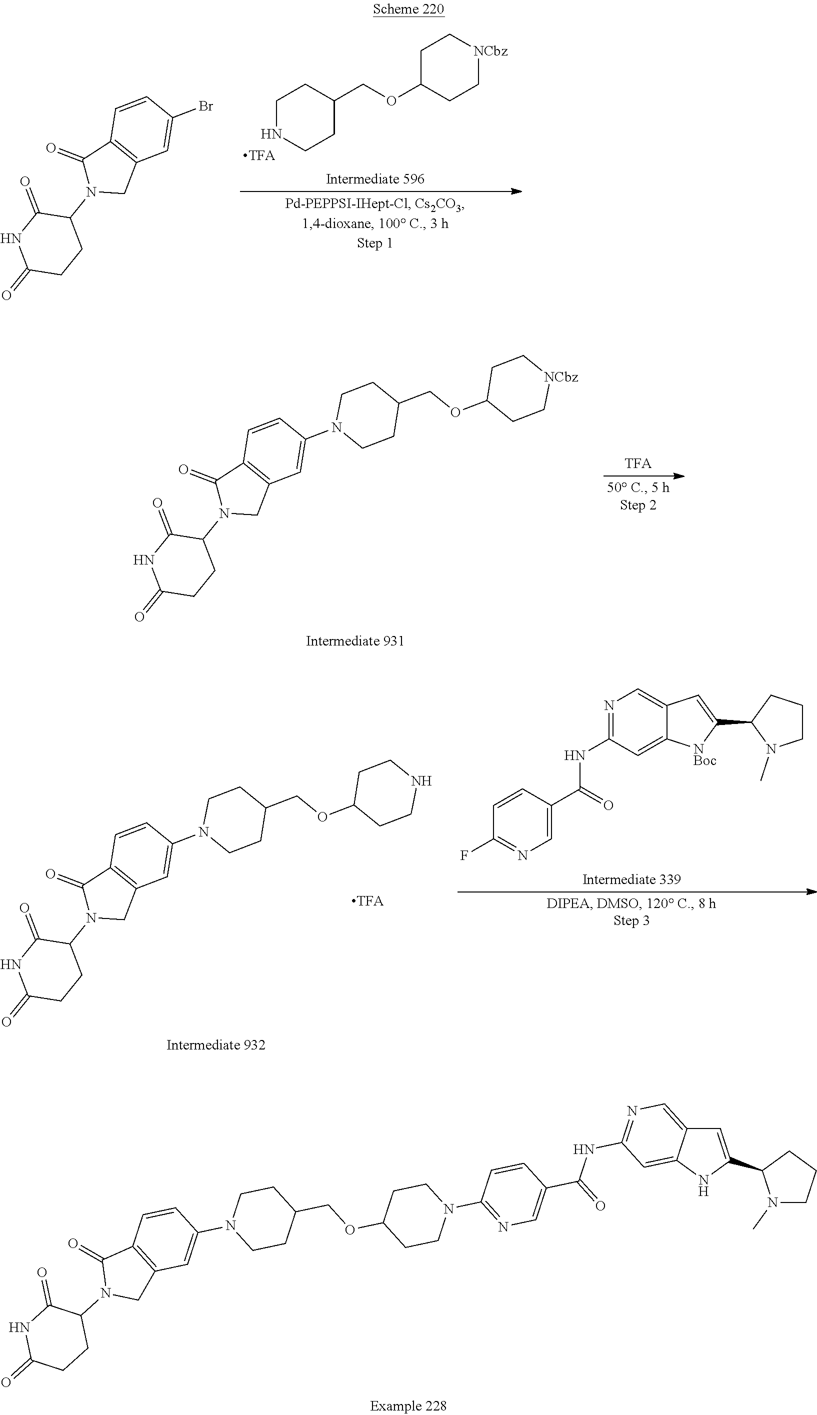 [SMILES: O=C1CCC(N2Cc3cc(Br)ccc3C2=O)C(=O)N1 ; O=C(OCc1ccccc1)N1CCC(OCC2CCNCC2)CC1 ; O=C1CCC(N2Cc3cc(N4CCC(COC5CCN(C(=O)OCc6ccccc6)CC5)CC4)ccc3C2=O)C(=O)N1 ; O=C1CCC(N2Cc3cc(N4CCC(COC5CCNCC5)CC4)ccc3C2=O)C(=O)N1 ; CN1CCC[C@@H]1c1cc2cnc(NC(=O)c3ccc(F)nc3)cc2n1C(=O)OC(C)(C)C ; CN1CCC[C@@H]1c1cc2cnc(NC(=O)c3ccc(N4CCC(OCC5CCN(c6ccc7c(c6)CN(C6CCC(=O)NC6=O)C7=O)CC5)CC4)nc3)cc2[nH]1]

### Step 1

### Intermediate 931: tert-butyl benzyl 4-((1-(2-(2,6-dioxopiperidin-3-yl)-1-oxoisoindolin-5-yl)piperidin-4-yl)methoxy)piperidine-1-carboxylate

To a solution of 3-(5-bromo-1-oxoisoindolin-2-yl)piperidine-2,6-dione (CAS No. 1010100-26-1, 1.0 g, 3.09 mmol) 1,4-dioxane (10 mL) was added benzyl 4-(piperidin-4-ylmethoxy)piperidine-1-carboxylate trifluoroacetate (Intermediate 596, 2.76 g, 6.19 mmol) followed by cesium carbonate (2.02 g, 6.19 mmol). The reaction mixture was purged with argon for 15 min then Pd-PEPPSI-IHept-Cl (0.152 g, 0.15 mmol) was added. The reaction mixture was purged with argon for another 10 min and stirred at 100° C. for 3 h. The reaction mixture was concentrated in vacuo. The crude material was purified by combi-flash chromatography, by eluting with 80-100% ethyl acetate in heptane, to afford tert-butyl benzyl 4-((1-(2-(2,6-dioxopiperidin-3-yl)-1-oxoisoindolin-5-yl)piperidin-4-yl)methoxy)piperidine-1-carboxylate (Intermediate 931, 0.73 g, 41%) as a yellow solid. ¹H NMR (400 MHz, DMSO-d₆) δ ppm 1.20-1.27 (m, 2H), 1.34-1.40 (m, 2H), 1.75-1.80 (m, 5H), 1.92-1.99 (m, 1H), 2.31-2.42 (m, 3H), 2.53-2.61 (m, 1H), 2.78-2.86 (m, 3H), 3.14-3.20 (m, 2H), 3.23-3.27 (m, 2H), 3.43-3.48 (m, 1H), 3.60-3.63 (m, 2H), 3.84-3.87 (m, 2H), 4.17-4.21 (m, 1H), 4.28-4.32 (m, 1H), 5.03-5.06 (m, 1H), 7.02-7.04 (m, 2H), 7.33-7.36 (m, 5H), 7.46-7.49 (m, 1H), 10.93 (s, 1H). Mass spec m/z 575.1 [M+H]⁺.

### Step 2

### Intermediate 932: 3-(1-oxo-5-(4-((piperidin-4-yloxy)methyl)piperidin-1-yl)isoindolin-2-yl)piperidine-2,6-dione trifluoroacetate

A solution of tert-butyl benzyl 4-((1-(2-(2,6-dioxopiperidin-3-yl)-1-oxoisoindolin-5-yl)piperidin-4-yl)methoxy)piperidine-1-carboxylate (Intermediate 931, 0.8 g, 1.0 mmol) in trifluoracetic acid (45 mL) was stirred at 50° C. for 5 h. The reaction mixture was concentrated in vacuo to afford 3-(1-oxo-5-(4-((piperidin-4-yloxy)methyl)piperidin-1-yl)isoindolin-2-yl)piperidine-2,6-dione trifluoroacetate (Intermediate 932, 1.2 g) as a brown solid which was used for the next step without further purification. Mass spec m/z 441.3 [M+H]⁺.

### Step 3

### Example 228: 6-(4-((1-(2-(2,6-dioxopiperidin-3-yl)-1-oxoisoindolin-5-yl)piperidin-4-yl)methoxy)piperidin-1-yl)-N-(2-((R)-1-methylpyrrolidin-2-yl)-1H-pyrrolo[3,2-c]pyridin-6-yl)nicotinamide

To a solution of 3-(1-oxo-5-(4-((piperidin-4-yloxy)methyl)piperidin-1-yl)isoindolin-2-yl)piperidine-2,6-dione trifluoroacetate (Intermediate 932, 0.3 g, 0.54 mmol) in dimethyl sulfoxide (4.0 mL) was added tert-butyl (R)-6-(6-fluoronicotinamido)-2-(1-methylpyrrolidin-2-yl)-1H-pyrrolo[3,2-c]pyridine-1-carboxylate (Intermediate 339, 0.237 g, 0.54 mmol) followed by N,N-diisopropylethylamine (0.87 mL, 4.8 mmol). The reaction mixture was heated at 120° C. for 8 h. The reaction mixture was quenched with water (50 mL), the resultant precipitate was collected by filtration and dried in vacuo. The crude material was purified by preparative HPLC (Method A) to afford 6-(4-((1-(2-(2,6-dioxopiperidin-3-yl)-1-oxoisoindolin-5-yl)piperidin-4-yl)methoxy)piperidin-1-yl)-N-(2-((R)-1-methylpyrrolidin-2-yl)-1H-pyrrolo[3,2-c]pyridin-6-yl)nicotinamide (Example 228, 0.025 g, 5%) as an off-white solid. ¹H NMR (400 MHz, DMSO-d₆) δ ppm 1.21-1.32 (m, 2H), 1.42-1.49 (m, 2H), 1.72-1.99 (m, 9H), 2.12-2.18 (m, 4H), 2.21-2.28 (m, 1H), 2.36-2.41 (m, 1H), 2.53-2.61 (m, 2H), 2.79-2.94 (m, 3H), 3.12-3.16 (m, 1H), 3.37-3.40 (m, 4H), 3.53-3.60 (m, 1H), 3.85-3.92 (m, 2H), 3.95-4.06 (m, 2H), 4.17-4.21 (m, 1H), 4.29-4.33 (m, 1H), 5.01-5.06 (m, 1H), 6.37 (s, 1H), 6.88 (d, J=8.8 Hz, 1H), 7.02-7.05 (m, 2H), 7.49 (d, J=8.8 Hz, 1H), 8.13-8.16 (m, 2H), 8.48 (s, 1H), 8.78 (s, 1H), 10.25 (s, 1H), 10.92 (s, 1H), 11.32 (s, 1H). Mass spec m/z 760.3 [M+H]⁺.

### Example 229 was Prepared Following Scheme 221

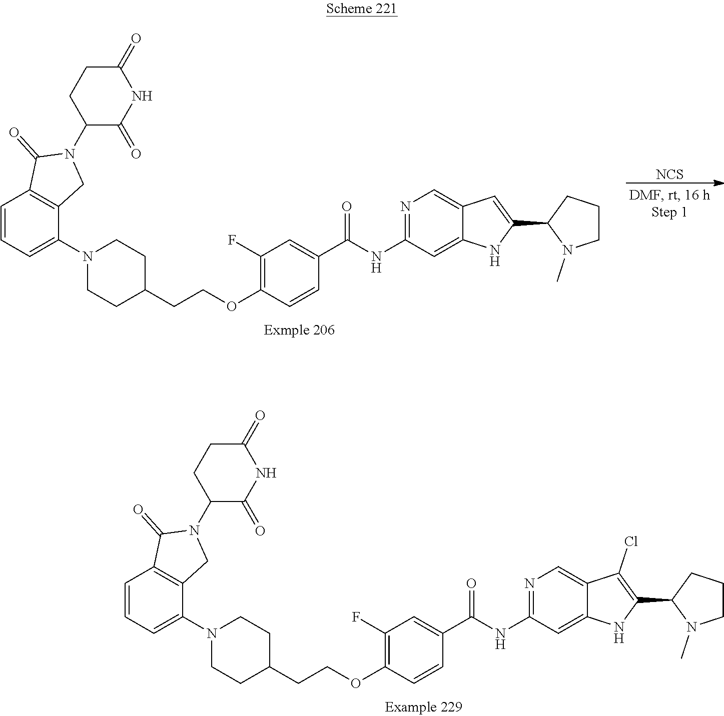 [SMILES: CN1CCC[C@@H]1c1cc2cnc(NC(=O)c3ccc(OCCC4CCN(c5cccc6c5CN(C5CCC(=O)NC5=O)C6=O)CC4)c(F)c3)cc2[nH]1 ; "NCS" ; CN1CCC[C@@H]1c1[nH]c2cc(NC(=O)c3ccc(OCCC4CCN(c5cccc6c5CN(C5CCC(=O)NC5=O)C6=O)CC4)c(F)c3)ncc2c1Cl]

### Step 1

### Example 229: N-(3-chloro-2-((R)-1-methylpyrrolidin-2-yl)-1H-pyrrolo[3,2-c]pyridin-6-yl)-4-(2-(1-(2-(2,6-dioxopiperidin-3-yl)-1-oxoisoindolin-4-yl)piperidin-4-yl)ethoxy)-3-fluorobenzamide

To a solution of 4-(2-(1-(2-(2,6-dioxopiperidin-3-yl)-1-oxoisoindolin-4-yl)piperidin-4-yl)ethoxy)-3-fluoro-N-(2-((R)-1-methylpyrrolidin-2-yl)-1H-pyrrolo[3,2-c]pyridin-6-yl)benzamide (Example 206, 0.080 g, 0.11 mmol) in dimethylformamide (5 mL) was added N-chlorosuccinimide (0.023 g, 0.16 mmol) at room temperature. The reaction mixture was stirred at room temperature for 16 h. After completion, the reaction mixture was quenched with ice cold water (10 mL), solid was precipitated out, which was collected by filtration and dried in vacuo. The crude material was purified by preparative HPLC (Method A) to afford N-(3-chloro-2-((R)-1-methylpyrrolidin-2-yl)-1H-pyrrolo[3,2-c]pyridin-6-yl)-4-(2-(1-(2-(2,6-dioxopiperidin-3-yl)-1-oxoisoindolin-4-yl)piperidin-4-yl)ethoxy)-3-fluorobenzamide (Example 229, 0.001 g, 0.083% yield) as an off white solid. ¹H NMR (400 MHz, DMSO-d₆) δ ppm 1.32-1.41 (m, 2H), 1.61-1.70 (m, 1H), 1.74-1.87 (m, 6H), 1.94-2.00 (m, 2H), 2.14-2.19 (m, 4H), 2.27-2.33 (m, 1H), 2.44-2.46 (m, 1H), 2.56-2.61 (m, 1H), 2.71-2.79 (m, 2H), 2.89-2.91 (m, 1H), 3.15-3.19 (m, 1H), 3.31-3.42 (m, 2H), 3.50-3.54 (m, 1H), 4.23-4.32 (m, 3H), 4.42-4.46 (m, 1H), 5.09-5.13 (m, 1H), 7.17 (d, J=7.6 Hz, 1H), 7.29-7.34 (m, 2H), 7.43 (t, J=7.6 Hz, 1H), 7.93-7.96 (m, 2H), 8.22 (s, 1H), 8.50 (s, 1H), 10.57 (br s, 1H), 10.97 (br s, 1H), 11.71 (br s, 1H). Mass spec m/z 742.4 [M+H]⁺.

### Example 230 was Prepared Following Scheme 222

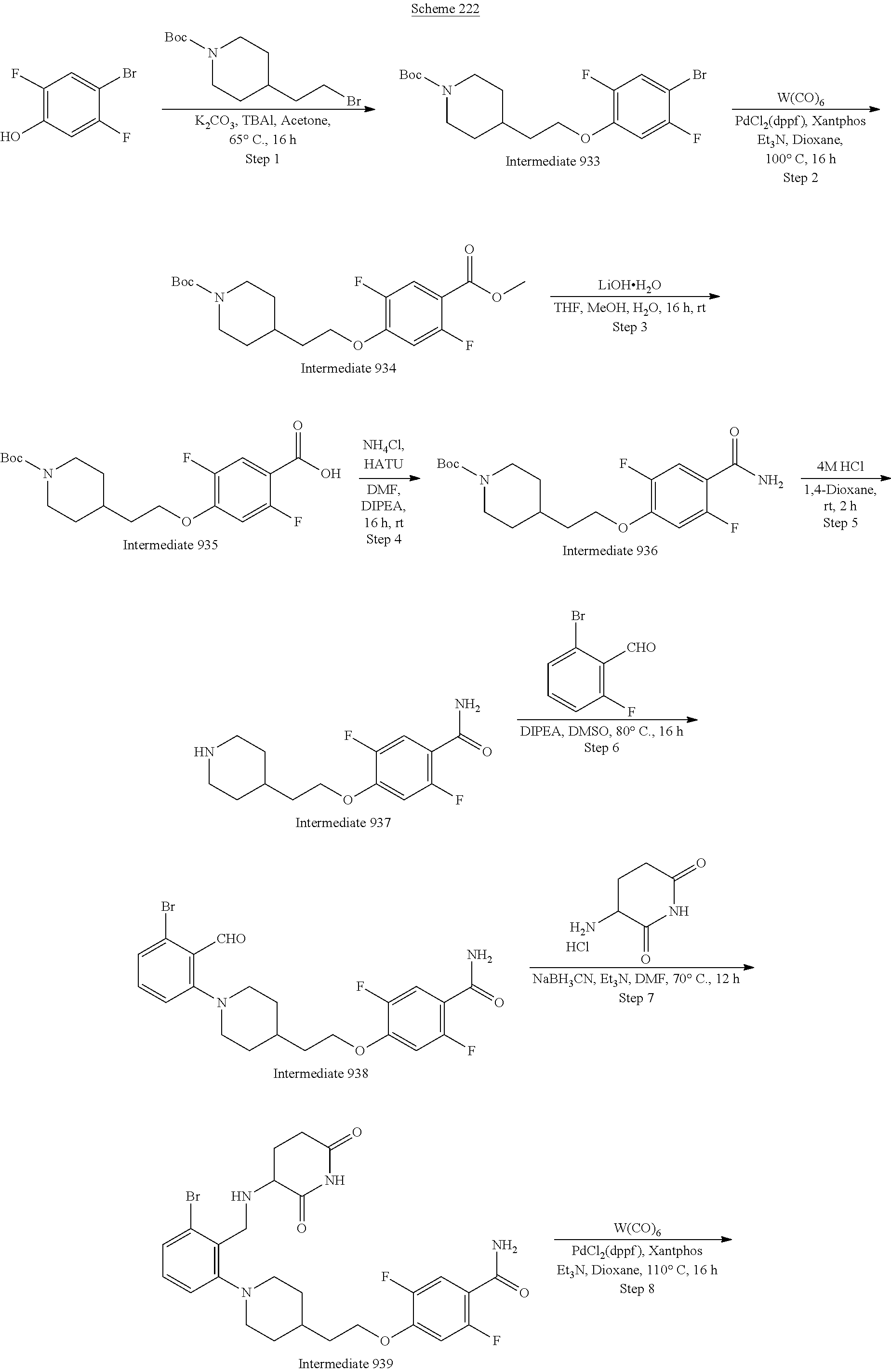

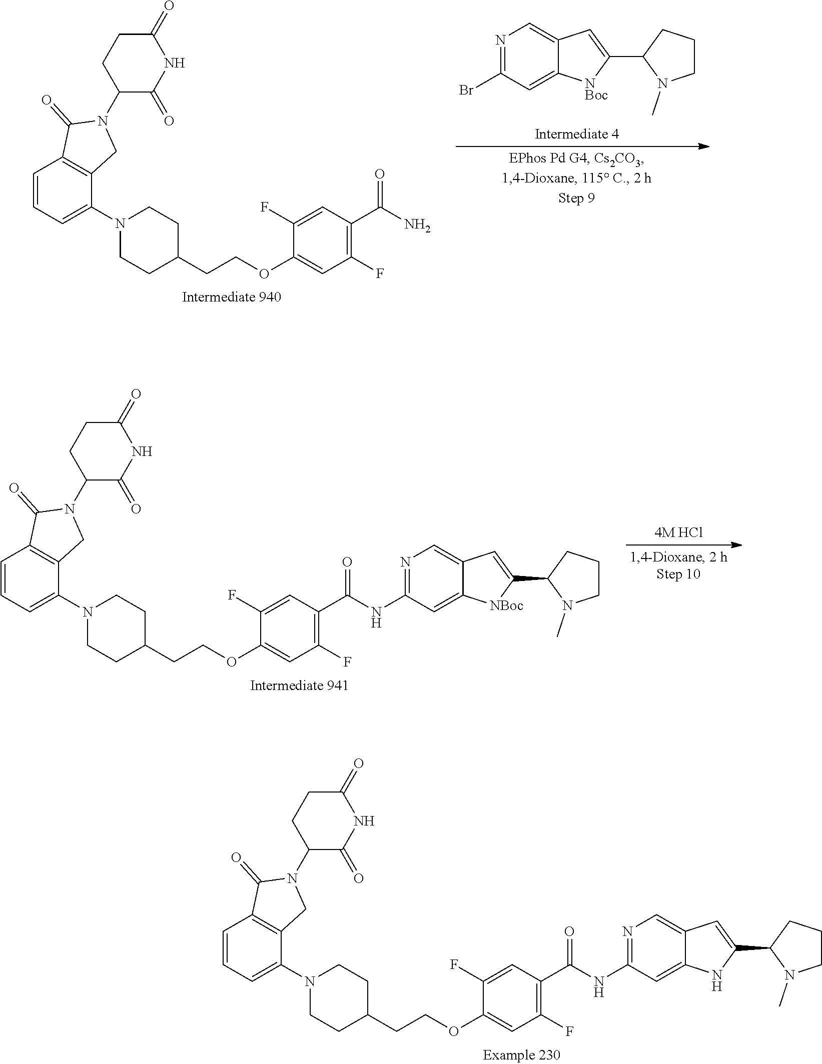 [SMILES: NC(=O)c1cc(F)c(OCCC2CCN(c3cccc4c3CN(C3CCC(=O)NC3=O)C4=O)CC2)cc1F ; CN1CCCC1c1cc2cnc(Br)cc2n1C(=O)OC(C)(C)C ; CN1CCC[C@@H]1c1cc2cnc(NC(=O)c3cc(F)c(OCCC4CCN(c5cccc6c5CN(C5CCC(=O)NC5=O)C6=O)CC4)cc3F)cc2n1C(=O)OC(C)(C)C ; CN1CCC[C@@H]1c1cc2cnc(NC(=O)c3cc(F)c(OCCC4CCN(c5cccc6c5CN(C5CCC(=O)NC5=O)C6=O)CC4)cc3F)cc2[nH]1]

### Step 1

### Intermediate 933: tert-butyl 4-(2-(4-bromo-2,5-difluorophenoxy)ethyl)piperidine-1-carboxylate

To a solution of 4-bromo-2,5-difluorophenol (CAS No. 486424-36-6, 14.30 g, 68.44 mmol) in acetone (200 mL) were added potassium carbonate (37.83 g, 273.78 mmol) followed by tetrabutylammonium iodide (5.15 g, 13.68 mmol) and tert-butyl 4-(2-bromoethyl)piperidine-1-carboxylate (CAS No. 169457-73-2, 20.0 g, 68.44 mmol) at room temperature. The reaction mixture was heated at 65° C. for 16 h. After completion, the reaction mixture was quenched with ice cold water (200 mL) and extracted with ethyl acetate (2×200 mL). The organic layer was separated, dried over anhydrous Na₂SO₄, filtered and concentrated in vacuo. The crude material was purified by combiflash chromatography by eluting with 20% ethyl acetate in heptane to afford tert-butyl 4-(2-(4-bromo-2,5-difluorophenoxy)ethyl)piperidine-1-carboxylate (Intermediate 933, 21.0 g, 73% yield) as white solid. ¹H NMR (400 MHz, DMSO-d₆) δ ppm 0.83-0.87 (m, 2H), 1.01-1.08 (m, 2H), 1.21-1.24 (m 3H), 1.39 (s, 9H), 1.64-1.68 (m, 4H), 2.67-2.68 (m, 2H), 7.37 (dd, J=10.45, 7.52 Hz, 1H), 7.68 (dd, J=10.64, 6.60 Hz, 1H). Mass spec m/z 363.8 [M−56+H]⁺.

### Step 2

### Intermediate 934: tert-butyl 4-(2-(2,5-difluoro-4-(methoxycarbonyl)phenoxy)ethyl)piperidine-1-carboxylate

To a solution of tert-butyl 4-(2-(4-bromo-2,5-difluorophenoxy)ethyl)piperidine-1-carboxylate (Intermediate 933, 5.0 g, 11.90 mmol) in 1,4-dioxane (40 mL) and methanol (10 mL) was added triethylamine (5.0 mL, 35.69 mmol). The reaction mixture was purged with argon for 15 min then Xantphos (1.06 g, 1.78 mmol) followed by PdCl₂(dppf) (1.31 g, 1.78 mmol) and tungsten hexacarbonyl (2.09 g, 5.94 mmol) were added at room temperature. The reaction mixture was further purged with argon for 10 min and heated at 100° C. for 16 h. Progress of the reaction was monitored by TLC. After completion, the reaction mixture was filtered through a celite bed and filter cake was washed with ethyl acetate (200 mL), filtrate was concentrated in vacuo. The crude material was purified by combi-flash chromatography by eluting with 20% ethyl acetate in heptane to afford tert-butyl 4-(2-(2,5-difluoro-4-(methoxycarbonyl)phenoxy)ethyl)piperidine-1-carboxylate (Intermediate 934, 3.7 g, 47% yield) as an off white solid. ¹H NMR (400 MHz, DMSO-d₆) δ ppm 1.00-1.12 (m, 2H), 1.39 (s, 9H), 1.63-1.71-1.74 (m, 5H), 2.64-2.76 (m, 2H), 3.82 (s, 3H), 3.91-3.93 (m, 2H), 4.17-4.20 (m, 2H), 7.27 (dd, J=12.47, 6.97 Hz, 1H), 7.65 (dd, J=11.37, 6.97 Hz, 1H). Mass spec m/z 299.9 [M−100+H]⁺.

### Step 3

### Intermediate 935: 4-(2-(1-(tert-butoxycarbonyl)piperidin-4-yl)ethoxy)-2,5-difluorobenzoic acid

To a solution of tert-butyl 4-(2-(2,5-difluoro-4-(methoxycarbonyl)phenoxy)ethyl)piperidine-1-carboxylate (Intermediate 934, 9.8 g, 25.0 mmol) in a mixture of tetrahydrofuran (50 mL), methanol (50 mL), water (50 mL) was added lithium hydroxide (2.4 g, 98.0 mmol) at 0° C. and stirred at room temperature for 16 h. After completion, the reaction mixture was concentrated in vacuo. pH of the aqueous residue was adjusted to 4 with saturated solution of citric acid and the resulting solid precipitate was collected by filtration and dried in vacuo to afford 4-(2-(1-(tert-butoxycarbonyl)piperidin-4-yl)ethoxy)-2,5-difluorobenzoic acid (Intermediate 935, 9.5 g, crude) as pale yellow solid, which was used for the next step without further purification. ¹H NMR (400 MHz, DMSO-d₆) δ ppm 1.00-1.10 (m, 2H), 1.38 (s, 9H), 1.61-1.72 (m, 5H), 2.66-2.68 (m, 2H), 3.90-3.92 (m, 2H), 4.13-4.15 (m, 2H), 7.14 (dd, J=12.11, 6.97 Hz, 1H), 7.55 (dd, J=11.74, 6.97 Hz, 1H). Mass spec m/z 285.9 [M−100+H]⁺.

### Step 4

### Intermediate 936: tert-butyl 4-(2-(4-carbamoyl-2,5-difluorophenoxy)ethyl)piperidine-1-carboxylate

To a solution of 4-(2-(1-(tert-butoxycarbonyl)piperidin-4-yl)ethoxy)-2,5-difluorobenzoic acid (Intermediate 935, 9.80 g, 25.0 mmol) in N,N-dimethylformamide (100 mL) were added HATU (15.0 g, 38.0 mmol) followed by N,N-diisopropylethylamine (18.0 mL, 100.0 mmol) and ammonium chloride (5.4 g, 100.0 mmol) at room temperature. The resultant reaction mixture was stirred at room temperature for 16 h. After completion, the reaction mixture was quenched with water (300 mL), solid was precipitated out, which was collected by filtration and dried in vacuo to afford tert-butyl 4-(2-(4-carbamoyl-2,5-difluorophenoxy)ethyl)piperidine-1-carboxylate (Intermediate 936, 9.0 g, 74% yield, crude) as a white solid, which was used for the next step without further purification. ¹H NMR (400 MHz, DMSO-d₆) δ ppm 1.01-1.11 (m, 2H), 1.39 (s, 9H), 1.61-1.73 (m, 5H), 2.64-2.74 (m, 2H), 3.90-3.92 (m, 2H), 4.13-4.16 (m, 2H), 7.21 (dd, J=12.47, 6.97 Hz, 1H), 7.48-7.53 (m, 2H), 7.60 (br s, 1H). Mass spec m/z 383.0 [M−H]⁻.

### Step 5

### Intermediate 937: 2,5-difluoro-4-(2-(piperidin-4-yl)ethoxy)benzamide hydrochloride

To a solution of tert-butyl 4-(2-(4-carbamoyl-2,5-difluorophenoxy)ethyl)piperidine-1-carboxylate (Intermediate 936, 9.0 g, 23.4 mmol) in dichloromethane (95 mL) was added 4M hydrochloric acid in 1,4-dioxane (95 mL) at 0° C. The reaction mixture was stirred at room temperature for 2 h. After completion, the reaction mixture was concentrated in vacuo to afford 2,5-difluoro-4-(2-(piperidin-4-yl)ethoxy)benzamide hydrochloride (Intermediate 937, 8.5 g, 93%, yield) as an off white solid, which was used for the next step without further purification. ¹H NMR (401 MHz, DMSO-d₆) δ ppm 1.34-1.46 (m, 2H), 1.71-1.91 (m, 5H), 2.78-2.89 (m, 2H), 3.21-3.24 (m, 2H), 4.14-4.17 (m, 2H), 7.21 (dd, J=12.26, 7.00 Hz, 1H), 7.50-7.64 (m, 2H), 8.89 (br s, 1H), 9.10 (br s, 1H). Mass spec m/z 285.1 [M+H]⁺.

### Step 6

### Intermediate 938: 4-(2-(1-(3-bromo-2-formylphenyl)piperidin-4-yl)ethoxy)-2,5-difluorobenzamide

To a solution of 2,5-difluoro-4-(2-(piperidin-4-yl)ethoxy)benzamide hydrochloride (Intermediate 937, 8.5 g, 26 mmol) in dimethyl sulfoxide (90 mL) was added N,N-diisopropylethylamine (19 mL, 110.0 mmol) followed by 2-bromo-6-fluorobenzaldehyde (CAS No. 360575-28-6, 5.4 g, 26.0 mmol) at room temperature. The reaction mixture was heated at 80° C. for 16 h. After completion, the reaction mixture was quenched with water (250 mL) and extracted with ethyl acetate (2×125 mL). The organic layer was separated, dried over anhydrous Na₂SO₄, filtered and concentrated in vacuo. The crude material was purified by combi-flash chromatography by eluting with 100% ethyl acetate to afford 4-(2-(1-(3-bromo-2-formylphenyl)piperidin-4-yl)ethoxy)-2,5-difluorobenzamide (Intermediate 938, 5.8 g, 42% yield) as a yellow solid. ¹H NMR (400 MHz, DMSO-d₆) δ ppm 1.40-1.48 (m, 2H), 1.59-1.69 (m, 1H), 1.74-1.86 (m, 4H), 2.82-2.88 (m, 2H), 3.16-3.18 (m, 2H), 4.17-4.21 (m, 2H), 7.20-7.26 (m, 2H), 7.31 (d, J=7.70 Hz, 1H), 7.39-7.44 (m, 1H), 7.47-7.55 (m, 2H), 7.60 (br s, 1H), 10.06 (s, 1H). Mass spec m/z 469.0 [M+H+2]⁺.

### Step 7

### Intermediate 939: 4-(2-(1-(3-bromo-2-(((2,6-dioxopiperidin-3-yl)amino)methyl)phenyl)piperidin-4-yl)ethoxy)-2,5-difluorobenzamide

To a solution of 4-(2-(1-(3-bromo-2-formylphenyl)piperidin-4-yl)ethoxy)-2,5-difluorobenzamide (Intermediate 938, 4.8 g, 10.0 mmol) in acetonitrile (50 mL) were added 3-aminopiperidine-2,6-dione hydrochloride (CAS No. 24666-56-6, 2.0 g, 12.0 mmol) followed by triethylamine (4.3 mL, 31.0 mmol) at room temperature. The reaction mixture was stirred at room temperature for 2 h. Then sodium cyanoborohydride (2.0 g, 31.0 mmol) was added and heated at 70° C. for 12 h. After completion, the reaction mixture was quenched with water (100 mL) and extracted with ethyl acetate (3×100 mL). The organic layer was separated, dried over anhydrous Na₂SO₄, filtered, and concentrated in vacuo. The crude material was purified by combi-flash chromatography by eluting with 10% MeOH in DCM to afford 4-(2-(1-(3-bromo-2-(((2,6-dioxopiperidin-3-yl)amino)methyl)phenyl)piperidin-4-yl)ethoxy)-2,5-difluorobenzamide (Intermediate 939, 3.8 g, 59% yield) as an off white solid. ¹H NMR (400 MHz, DMSO-d₆) δ ppm 1.32-1.49 (m, 2H) 1.60-1.63 (m, 1H) 1.68-1.92 (m, 5H) 2.26-2.35 (m, 1H), 2.52-2.58 (m, 3H), 2.72-2.81 (m, 1H), 2.93-2.94 (m, 1H), 3.07-3.09 (m, 1H), 3.22-3.24 (m, 1H), 3.86-3.94 (m, 2H), 4.18-4.21 (m, 2H), 7.14-7.26 (m, 3H), 7.31-7.34 (m, 1H), 7.48-7.54 (m, 2H), 7.60 (br s, 1H), 10.76 (s, 1H). Mass spec m/z 581.1 [M+H+2]⁺.

### Step 8

### Intermediate 940: 4-(2-(1-(2-(2,6-dioxopiperidin-3-yl)-1-oxoisoindolin-4-yl)piperidin-4-yl)ethoxy)-2,5-difluorobenzamide

To a solution of 4-(2-(1-(3-bromo-2-(((2,6-dioxopiperidin-3-yl)amino)methyl)phenyl)piperidin-4-yl)ethoxy)-2,5-difluorobenzamide (Intermediate 939, 2.10 g, 3.62 mmol) in 1,4-dioxane (20 mL) was added triethylamine (1.52 mL, 10.9 mmol). The reaction mixture was purged with argon for 15 min then Xantphos (0.43 g, 0.72 mmol) followed by PdCl₂(dppf) (0.55 g, 0.72 mmol) and tungsten hexacarbonyl (0.82 g, 2.36 mmol) were added at room temperature. The reaction mixture was further purged with argon for 10 min and heated at 110° C. for 16 h. After completion, the reaction mixture was filtered through a celite bed and filer cake washed with ethyl acetate (200 mL), filtrate was concentrated in vacuo. The crude material was purified by combi-flash chromatography by eluting with 90% ethyl acetate in heptane to afford 4-(2-(1-(2-(2,6-dioxopiperidin-3-yl)-1-oxoisoindolin-4-yl)piperidin-4-yl)ethoxy)-2,5-difluorobenzamide (Intermediate 940, 0.75 g, 39% yield) as a brown solid. ¹H NMR (400 MHz, DMSO-d₆) δ ppm 1.30-1.43 (m, 2H), 1.59-1.86 (m, 5H), 1.96-2.03 (m, 1H), 2.40-2.47 (m, 1H), 2.60-2.78 (m, 3H), 2.83-2.94 (m, 1H), 3.34-3.36 (m, 2H), 4.16-4.18 (m, 2H), 4.26-4.32 (m, 1H), 4.40-4.46 (m, 1H), 5.06-5.07 (m, 1H), 7.15-7.22 (m, 2H), 7.30 (d, J=7.13 Hz, 1H), 7.40-7.45 (m, 1H), 7.50 (dd, J=11.63, 7.00 Hz, 1H). Mass spec m/z 527.6 [M+H]⁺.

### Step 9

### Intermediate 941: tert-butyl 6-(4-(2-(1-(2-(2,6-dioxopiperidin-3-yl)-1-oxoisoindolin-4-yl)piperidin-4-yl)ethoxy)-2,5-difluorobenzamido)-2-((R)-1-methylpyrrolidin-2-yl)-1H-pyrrolo[3,2-c]pyridine-1-carboxylate

To a solution of 4-(2-(1-(2-(2,6-dioxopiperidin-3-yl)-1-oxoisoindolin-4-yl)piperidin-4-yl)ethoxy)-2,5-difluorobenzamide (Intermediate 940, 0.1 g, 0.18 mmol) in 1,4-dioxane (2 mL) were added tert-butyl (R)-6-bromo-2-(1-methylpyrrolidin-2-yl)-1H-pyrrolo[3,2-c]pyridine-1-carboxylate (Intermediate 4, 0.09 g, 0.23 mmol) followed by cesium carbonate (0.015 g, 0.047 mmol) at room temperature. The reaction mixture was purged with argon for 15 min then EPhos Pd G4 (0.55 g, 0.56 mmol) was added. The reaction mixture was further purged with argon for 10 min and heated at 115° C. for 2 h. After completion, the reaction mixture was filtered through a celite bed, filter cake was washed with ethyl acetate (100 mL) and concentrated in vacuo. Crude residue was then washed with n-heptane (10 mL) and dried in vacuo to afford tert-butyl 6-(4-(2-(1-(2-(2,6-dioxopiperidin-3-yl)-1-oxoisoindolin-4-yl)piperidin-4-yl)ethoxy)-2,5-difluorobenzamido)-2-((R)-1-methylpyrrolidin-2-yl)-1H-pyrrolo[3,2-c]pyridine-1-carboxylate (Intermediate 941, 0.06 g, 14% yield) as a brown solid, which was used for the next step without further purification. Mass spec m/z 826.5 [M+H]⁺.

### Step 10

### Example 230: 4-(2-(1-(2-(2,6-dioxopiperidin-3-yl)-1-oxoisoindolin-4-yl)piperidin-4-yl)ethoxy)-2,5-difluoro-N-(2-((R)-1-methylpyrrolidin-2-yl)-1H-pyrrolo[3,2-c]pyridin-6-yl)benzamide

To a solution of tert-butyl 6-(4-(2-(1-(2-(2,6-dioxopiperidin-3-yl)-1-oxoisoindolin-4-yl)piperidin-4-yl)ethoxy)-2,5-difluorobenzamido)-2-((R)-1-methylpyrrolidin-2-yl)-1H-pyrrolo[3,2-c]pyridine-1-carboxylate (Intermediate 941, 0.3 g, 0.36 mmol) in dichloromethane (3 mL) was added 4M hydrochloric acid in 1,4-dioxane (3 mL) at 0° C. The reaction mixture was stirred at room temperature for 2 h. After completion, the reaction mixture was concentrated in vacuo. The crude material was purified by prep-HPLC in basic method to afford 4-(2-(1-(2-(2,6-dioxopiperidin-3-yl)-1-oxoisoindolin-4-yl)piperidin-4-yl)ethoxy)-2,5-difluoro-N-(2-((R)-1-methylpyrrolidin-2-yl)-1H-pyrrolo[3,2-c]pyridin-6-yl)benzamide (Example 230, 0.065 g, 25% yield) as an off white solid. ¹H NMR (400 MHz, DMSO-d₆) δ ppm 1.40-1.45 (m, 2H), 1.66-2.03 (m, 10H), 2.14-2.17 (m, 4H), 2.26-2.34 (m, 1H), 2.59-2.63 (m, 1H), 2.70-2.79 (m, 2H), 2.88-2.94 (m, 1H), 3.11-3.15 (m, 1H), 3.27-3.29 (m, 1H), 3.39-3.42 (m, 2H), 4.22-4.46 (m, 4H), 5.08-5.13 (m, 1H), 6.39 (s, 1H), 7.18 (d, J=7.75 Hz, 1H), 7.28-7.34 (m, 2H), 7.41-7.45 (m, 1H), 7.62 (dd, J=11.38, 7.00 Hz, 1H), 8.18 (s, 1H), 8.47 (br s, 1H) 10.12 (d, J=4.75 Hz, 1H), 11.00 (br s, 1H) 11.41 (br s, 1H). Mass spec m/z 726.2 [M+H]⁺.

### Example 231 was Prepared Following Scheme 223

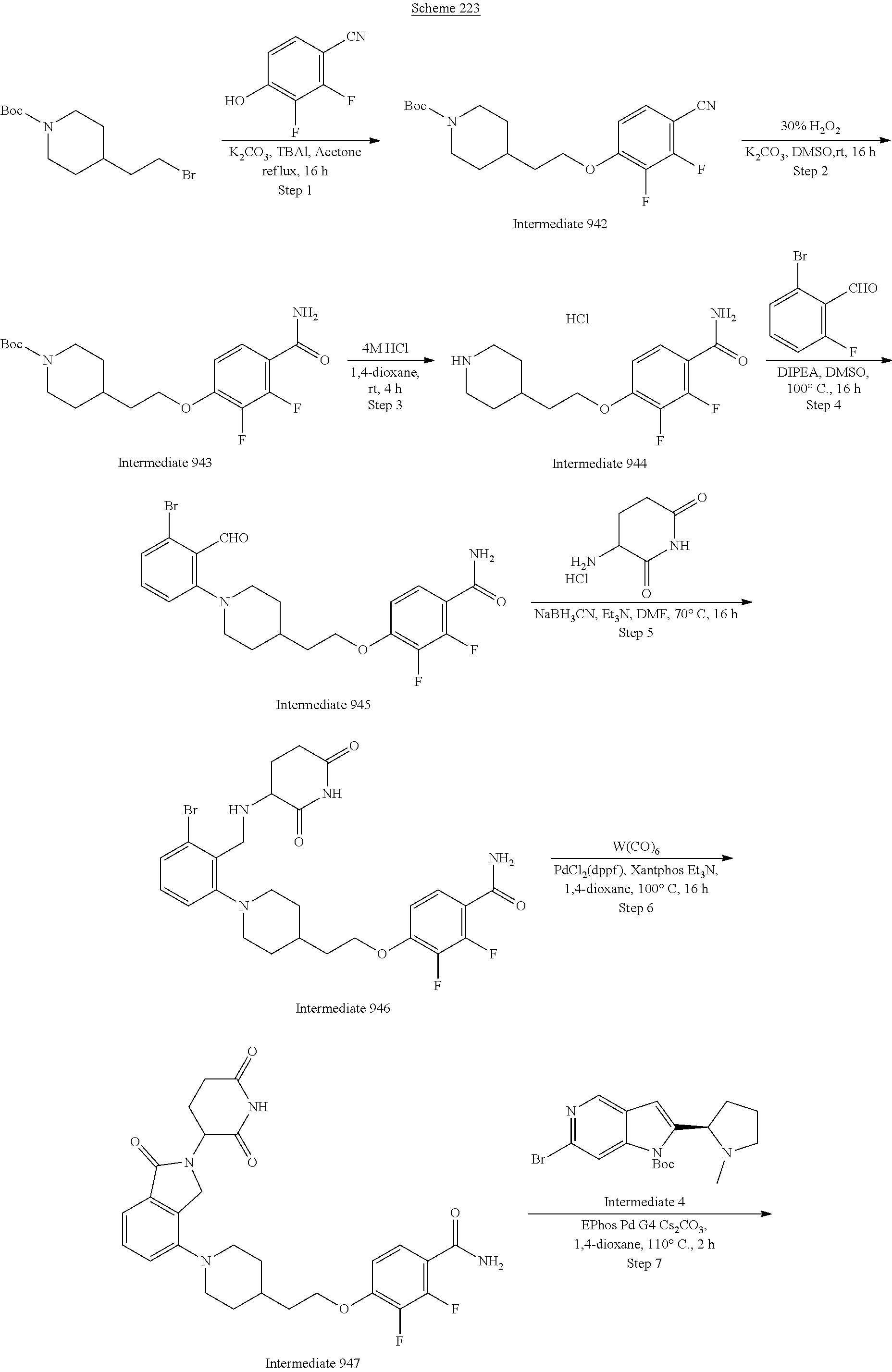

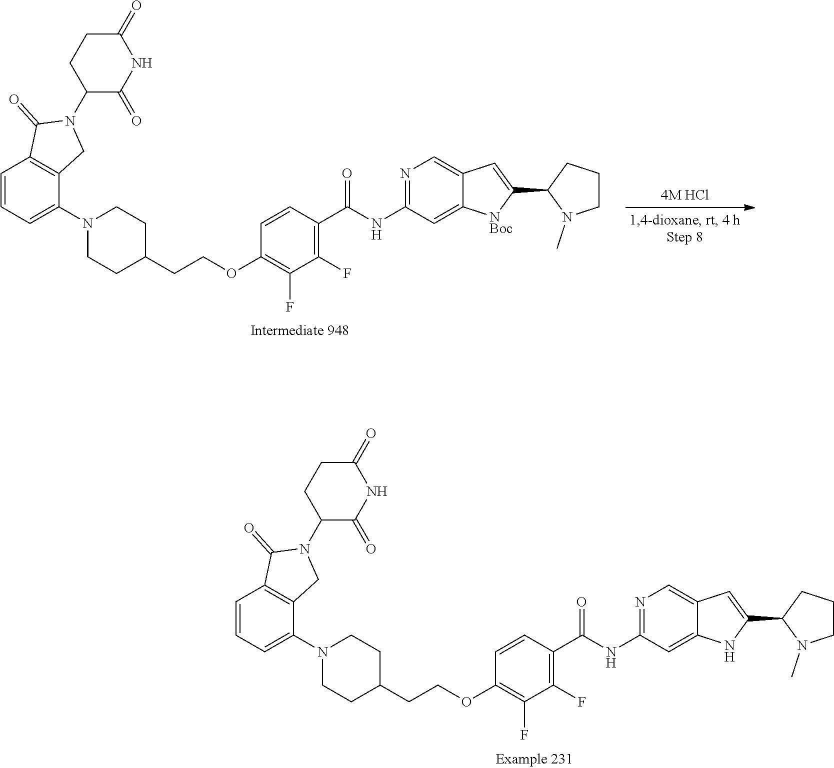 [SMILES: CN1CCC[C@@H]1c1cc2cnc(NC(=O)c3ccc(OCCC4CCN(c5cccc6c5CN(C5CCC(=O)NC5=O)C6=O)CC4)c(F)c3F)cc2n1C(=O)OC(C)(C)C ; CN1CCC[C@@H]1c1cc2cnc(NC(=O)c3ccc(OCCC4CCN(c5cccc6c5CN(C5CCC(=O)NC5=O)C6=O)CC4)c(F)c3F)cc2[nH]1]

### Step 1

### Intermediate 942: tert-butyl 4-(2-(4-cyano-2,3-difluorophenoxy)ethyl)piperidine-1-carboxylate

To a solution of 2,3-difluoro-4-hydroxybenzonitrile (CAS No. 126162-38-7, 7.43 g, 47.91 mmol) in acetone (50 mL) was added potassium carbonate (26.5 g, 191.6 mmol), tert-butyl 4-(2-bromoethyl)piperidine-1-carboxylate (CAS No. 169457-73-2, 14.0 g, 47.9 mmol) and tetrabutylammonium iodide (3.61 g, 9.58 mmol). The reaction mixture was heated at 50° C. for 16 h then concentrated in vacuo. The crude material was purified by combi-flash chromatography, by eluting with 30% ethyl acetate in heptane, to afford tert-butyl 4-(2-(4-cyano-2,3-difluorophenoxy)ethyl)piperidine-1-carboxylate (Intermediate 942, 15.0 g, 85%) as an off-white solid. ¹H NMR (400 MHz, DMSO-d₆) δ ppm 1.00-1.12 (m, 2H), 1.38 (s, 9H), 1.62-1.76 (m, 5H), 2.67-2.69 (m, 2H), 3.90-3.92 (m, 2H), 4.22-4.26 (m, 2H), 7.25 (t, J=7.89 Hz, 1H), 7.73 (t, J=7.52 Hz, 1H).

### Step 2

### Intermediate 943: tert-butyl 4-(2-(4-carbamoyl-2,3-difluorophenoxy)ethyl)piperidine-1-carboxylate

To a cooled (0° C.) solution of tert-butyl 4-(2-(4-cyano-2,3-difluorophenoxy)ethyl)piperidine-1-carboxylate (Intermediate 942, 11.0 g, 30.0 mmol) in dimethyl sulfoxide (40 mL) was added potassium carbonate (10.4 g, 75.0 mmol) followed by 30% hydrogen peroxide (20.0 mL, 300 mmol) and the reaction mixture was stirred at room temperature for 16 h. The reaction mixture was then quenched with ice cold water (200 mL), the resultant precipitate was collected by filtration and dried in vacuo afford tert-butyl 4-(2-(4-carbamoyl-2,3-difluorophenoxy)ethyl)piperidine-1-carboxylate (Intermediate 943, 11.1 g, 96%) as an off-white solid, which was used for the next step without further purification. ¹H NMR (400 MHz, DMSO-d₆) δ ppm 1.03-1.12 (m, 2H), 1.39 (s, 9H), 1.59-1.69 (m, 5H), 2.65-2.67 (m, 2H), 3.90-3.92 (m, 2H), 4.16-4.19 (m, 2H), 7.10 (t, J=7.89 Hz, 1H), 7.46 (t, J=8.07 Hz, 1H), 7.61 (d, J=7.34 Hz, 2H). Mass spec m/z 383.1 [M−H]⁻.

### Step 3

### Intermediate 944: 2,3-difluoro-4-(2-(piperidin-4-yl)ethoxy)benzamide hydrochloride

To a cooled (0° C.) solution of tert-butyl 4-(2-(4-carbamoyl-2,3-difluorophenoxy)ethyl)piperidine-1-carboxylate (Intermediate 943, 11.0 g, 28.6 mmol) in dichloromethane (110 mL) was added 4M hydrochloric acid in 1,4-dioxane (110 mL) and the reaction mixture was stirred at room temperature for 4 h. The reaction mixture was then concentrated in vacuo to afford 2,3-difluoro-4-(2-(piperidin-4-yl)ethoxy)benzamide hydrochloride (Intermediate 944, 11.5 g) as an off-white solid, which was used for the next step without further purification. ¹H NMR (400 MHz, DMSO-d₆) δ ppm 1.31-1.38 (m, 2H), 1.71-1.78 (m, 3H), 1.84-1.86 (m, 2H), 2.83-2.85 (m, 2H), 3.22-3.25 (m, 2H), 3.56-3.57 (m, 2H), 4.17-4.20 (m, 2H), 7.10 (t, J=8.07 Hz, 1H), 7.47 (t, J=7.52 Hz, 1H), 8.53 (br s, 1H), 8.74 (br s, 1H). Mass spec m/z 285.1 [M+H]⁺.

### Step 4

### Intermediate 945: 4-(2-(1-(3-bromo-2-formylphenyl)piperidin-4-yl)ethoxy)-2,3-difluorobenzamide

To a solution of 2,3-difluoro-4-(2-(piperidin-4-yl)ethoxy)benzamide hydrochloride (Intermediate 944, 11.0 g, 34.3 mmol) in dimethylformamide (110 mL) was added potassium carbonate (19.0 g, 137 mmol) followed by 2-bromo-6-fluorobenzaldehyde (CAS No. 360575-28-6, 6.96 g, 34.3 mmol) and the reaction mixture was heated at 100° C. for 16 h. The reaction mixture was then quenched with water (250 mL) and extracted with ethyl acetate (2×250 mL). The combined organic layers were dried over anhydrous Na₂SO₄, filtered and concentrated in vacuo to afford 4-(2-(1-(3-bromo-2-formylphenyl)piperidin-4-yl)ethoxy)-2,3-difluorobenzamide (Intermediate 945, 11.0 g, 69%) as a pale yellow solid, which was used for the next step without further purification. ¹H NMR (400 MHz, DMSO-d₆) δ ppm 1.36-1.47 (m, 2H), 1.64-1.84 (m, 5H), 2.83-2.86 (m, 2H), 3.14-3.16 (m, 2H), 4.18-4.24 (m, 2H), 7.11 (t, J=8.02 Hz, 1H), 7.21 (d, J=7.83 Hz, 1H), 7.30 (d, J=7.43 Hz, 1H), 7.37-7.48 (m, 2H), 7.56-7.59 (m, 2H), 10.02 (s, 1H). Mass spec m/z 469.1 [M+H+2]⁺.

### Step 5

### Intermediate 946: 4-(2-(1-(3-bromo-2-(((2,6-dioxopiperidin-3-yl)amino)methyl)phenyl)piperidin-4-yl)ethoxy)-2,3-difluorobenzamide

To a solution of 4-(2-(1-(3-bromo-2-formylphenyl)piperidin-4-yl)ethoxy)-2,3-difluorobenzamide (Intermediate 945, 8.0 g, 17.1 mmol) in acetonitrile (30 mL) was added 3-aminopiperidine-2,6-dione hydrochloride (CAS No. 24666-56-6, 2.81 g, 17.1 mmol) and the reaction mixture was stirred at room temperature for 2 h. Sodium cyanoborohydride (3.79 g, 51.4 mmol) was then added and the reaction mixture was heated at 70° C. for 16 h. The reaction mixture was quenched with water (100 mL) and extracted with ethyl acetate (3×100 mL). The combined organic layers were dried over anhydrous Na₂SO₄, filtered, and concentrated in vacuo. The crude material was purified by combi-flash chromatography, by eluting with 10% MeOH in DCM, to afford 4-(2-(1-(3-bromo-2-(((2,6-dioxopiperidin-3-yl)amino)methyl)phenyl)piperidin-4-yl)ethoxy)-2,3-difluorobenzamide (Intermediate 946, 5.0 g, 50%) as a white solid. ¹H NMR (400 MHz, DMSO-d₆) δ ppm 1.08-1.19 (m, 2H), 1.36-1.46 (m, 2H), 1.64-1.87 (m, 6H), 2.27-2.34 (m, 1H), 2.53-2.58 (m, 3H), 2.77-2.84 (m, 1H), 2.93-2.95 (m, 1H), 3.06-3.08 (m, 1H), 3.22-3.24 (m, 1H), 3.84-3.99 (m, 1H), 4.21-4.26 (m, 2H), 7.10-7.18 (m, 3H), 7.33 (d, J=6.60 Hz, 1H), 7.47 (t, J=7.89 Hz, 1H), 7.60 (d, J=6.60 Hz, 2H), 10.80 (s, 1H). Mass spec m/z 579.0 [M+H]⁺.

### Step 6

### Intermediate 947: 4-(2-(1-(2-(2,6-dioxopiperidin-3-yl)-1-oxoisoindolin-4-yl)piperidin-4-yl)ethoxy)-2,3-difluorobenzamide

To a solution of 4-(2-(1-(3-bromo-2-(((2,6-dioxopiperidin-3-yl)amino)methyl)phenyl)piperidin-4-yl)ethoxy)-2,3-difluorobenzamide (Intermediate 946, 3.0 g, 5.17 mmol) in 1,4-dioxane (20 mL) was added triethylamine (2.07 mL, 15.5 mmol). The reaction mixture was purged with argon for 15 min then Xantphos (0.46 g, 0.77 mmol) followed by PdCl₂(dppf) (0.59 g, 0.77 mmol) and tungsten hexacarbonyl (0.92 g, 2.58 mmol) were added. The reaction mixture was further purged with argon for 10 min and heated at 100° C. for 16 h. The reaction mixture was filtered through a celite bed and the filter pad was washed with ethyl acetate (200 mL). The filtrate was concentrated in vacuo and the crude material purified by combi-flash chromatography, by eluting with 10% MeOH in DCM, to afford 4-(2-(1-(2-(2,6-dioxopiperidin-3-yl)-1-oxoisoindolin-4-yl)piperidin-4-yl)ethoxy)-2,3-difluorobenzamide (Intermediate 947, 1.7 g, 62%) as a brown solid. ¹H NMR (400 MHz, DMSO-d₆) δ ppm 1.35-1.37 (m, 2H), 1.76-1.89 (m, 6H), 1.96-2.03 (m, 1H), 2.57-2.59 (m, 1H), 2.70-2.75 (m, 2H), 2.87-2.96 (m, 1H), 3.36-3.44 (m, 2H), 4.22-4.47 (m, 4H), 5.10-5.12 (m, 1H), 7.12-7.18 (m, 2H), 7.29-7.30 (m, 1H), 7.41-7.47 (m, 2H), 7.59 (d, J=6.97 Hz, 2H), 10.97 (s, 1H). Mass spec m/z 527.0 [M+H]⁺.

### Step 7

### Intermediate 948: tert-butyl 6-(4-(2-(1-(2-(2,6-dioxopiperidin-3-yl)-1-oxoisoindolin-4-yl)piperidin-4-yl)ethoxy)-2,3-difluorobenzamido)-2-((R)-1-methylpyrrolidin-2-yl)-1H-pyrrolo[3,2-c]pyridine-1-carboxylate

To a solution of 4-(2-(1-(2-(2,6-dioxopiperidin-3-yl)-1-oxoisoindolin-4-yl)piperidin-4-yl)ethoxy)-2,3-difluorobenzamide (Intermediate 947, 0.05 g, 0.094 mmol) in 1,4-dioxane (1 mL) was added tert-butyl (R)-6-bromo-2-(1-methylpyrrolidin-2-yl)-1H-pyrrolo[3,2-c]pyridine-1-carboxylate (Intermediate 4, 0.039 g, 1.04 mmol) followed by cesium carbonate (0.092 g, 0.28 mmol). The reaction mixture was purged with argon for 15 min then EPhos Pd G4 (0.026 g, 0.028 mmol) was added. The reaction mixture was further purged with argon for 10 min and heated at 110° C. for 2 h. The reaction mixture was filtered through a celite bed, the filter pad was washed with ethyl acetate (100 mL) and the filtrate concentrated in vacuo. The crude material was purified by combi-flash chromatography, by eluting with 9% MeOH in DCM, to afford tert-butyl 6-(4-(2-(1-(2-(2,6-dioxopiperidin-3-yl)-1-oxoisoindolin-4-yl)piperidin-4-yl)ethoxy)-2,3-difluorobenzamido)-2-((R)-1-methylpyrrolidin-2-yl)-1H-pyrrolo[3,2-c]pyridine-1-carboxylate (Intermediate 948, 0.045 g, 58%) as an off-white solid. Mass spec m/z 826.5 [M+H]⁺.

### Step 8

### Example 231: 4-(2-(1-(2-(2,6-dioxopiperidin-3-yl)-1-oxoisoindolin-4-yl)piperidin-4-yl)ethoxy)-2,3-difluoro-N-(2-((R)-1-methylpyrrolidin-2-yl)-1H-pyrrolo[3,2-c]pyridin-6-yl)benzamide

To a cooled (0° C.) solution of tert-butyl 6-(4-(2-(1-(2-(2,6-dioxopiperidin-3-yl)-1-oxoisoindolin-4-yl)piperidin-4-yl)ethoxy)-2,3-difluorobenzamido)-2-((R)-1-methylpyrrolidin-2-yl)-1H-pyrrolo[3,2-c]pyridine-1-carboxylate (Intermediate 948, 0.3 g, 0.4 mmol) in dichloromethane (5 mL) was added 4M hydrochloric acid in 1,4-dioxane (3.0 mL) and the reaction mixture was stirred at room temperature for 4 h. The reaction mixture was concentrated in vacuo. The crude material was purified by preparative HPLC (Method A) to afford 4-(2-(1-(2-(2,6-dioxopiperidin-3-yl)-1-oxoisoindolin-4-yl)piperidin-4-yl)ethoxy)-2,3-difluoro-N-(2-((R)-1-methylpyrrolidin-2-yl)-1H-pyrrolo[3,2-c]pyridin-6-yl)benzamide (Example 231, 0.043 g, 20%) as an off-white solid. ¹H NMR (400 MHz, DMSO-d₆) δ ppm 1.38-1.44 (m, 2H), 1.65-1.68 (m, 1H), 1.78-1.91 (m, 8H), 1.97-2.01 (m, 1H), 2.14-2.21 (m, 4H), 2.23-2.29 (m, 1H), 2.56-2.60 (m, 1H), 2.71-2.79 (m, 2H), 2.86-2.93 (m, 1H), 3.11-3.15 (m, 1H), 3.29-3.31 (m, 1H), 3.35-3.42 (m, 2H), 4.24-4.28 (m, 3H), 4.42-4.46 (m, 1H), 5.08-5.12 (m, 1H), 6.39 (s, 1H), 7.15-7.19 (m, 2H), 7.30 (d, J=7.00 Hz, 1H), 7.41-7.45 (m, 1H), 7.51-7.55 (m, 1H), 8.17 (s, 1H), 8.48 (s, 1H), 10.31 (br s, 1H), 11.01 (br s, 1H), 11.41 (br s, 1H). Mass spec m/z 726.3 [M+H]⁺.

### Example 232 was Prepared Following Scheme 224

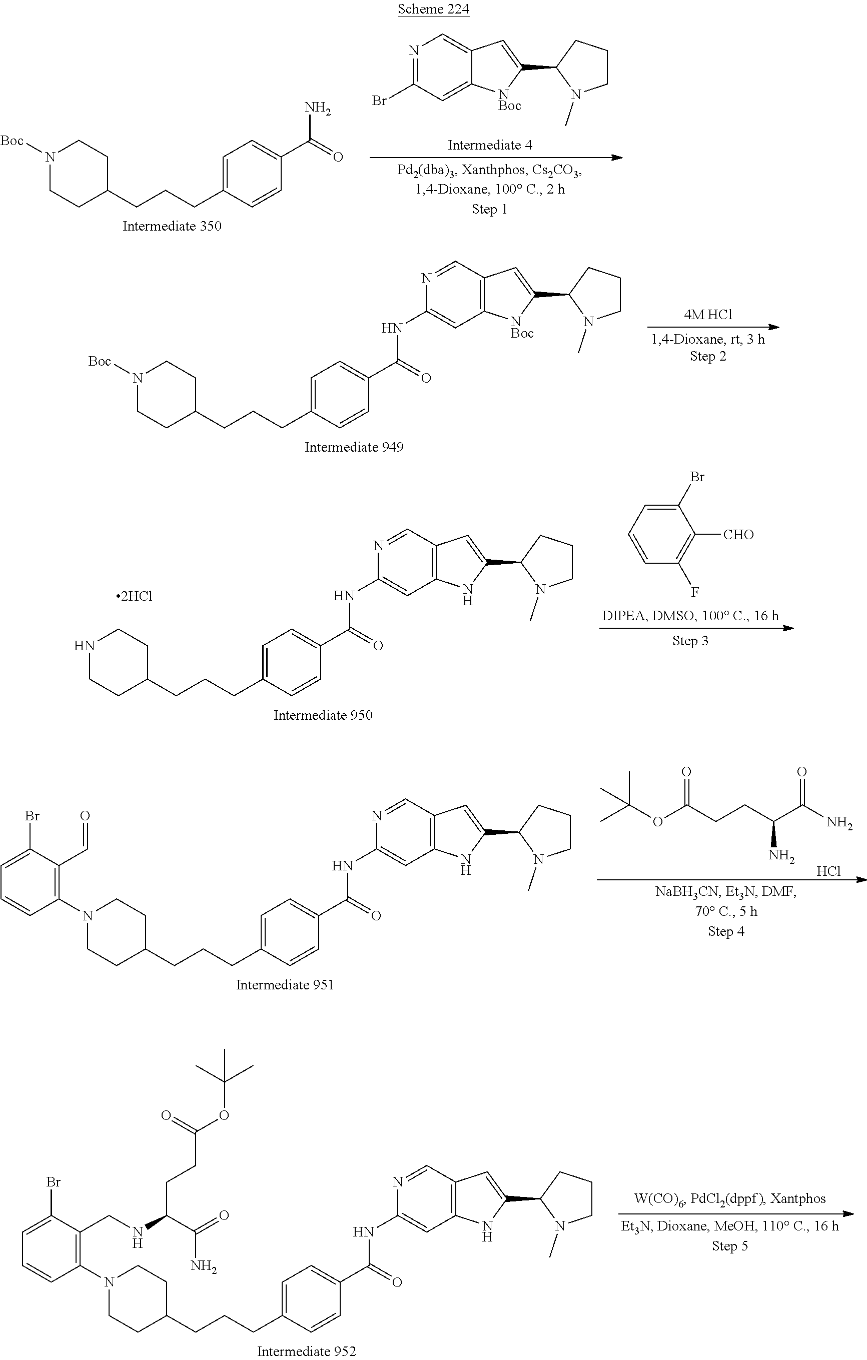

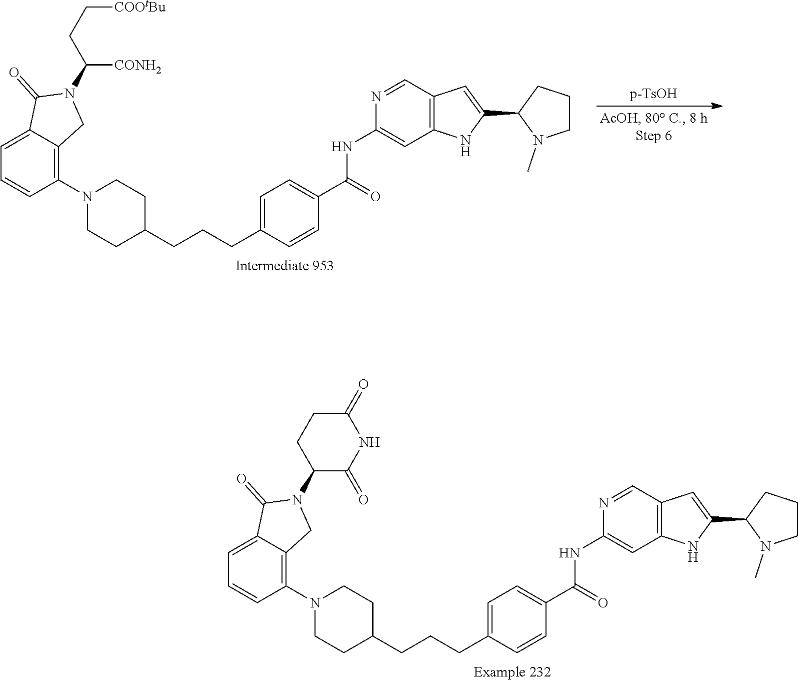

### Step 1

### Intermediate 949: tert-butyl (R)-6-(4-(3-(1-(tert-butoxycarbonyl)piperidin-4-yl)propyl)benzamido)-2-(1-methylpyrrolidin-2-yl)-1H-pyrrolo[3,2-c]pyridine-1-carboxylate

To a solution of tert-butyl 4-(3-(4-carbamoylphenyl)propyl)piperidine-1-carboxylate (Intermediate 350, 1.0 g, 2.89 mmol) in 1,4-dioxane (30 mL) was added tert-butyl (R)-6-bromo-2-(1-methylpyrrolidin-2-yl)-1H-pyrrolo[3,2-c]pyridine-1-carboxylate (Intermediate 4, 1.1 g, 2.88 mmol) followed by cesium carbonate (2.82 g, 8.66 mmol). The reaction mixture was purged with argon for 15 min then Xanthphos (0.50 g, 0.87 mmol) and Pd₂(dba)₃ (0.40 g, 0.43 mmol) were added. The reaction mixture was further purged with argon for 10 min and heated at 100° C. for 2 h. The reaction mixture was filtered through a celite bed, the filter pad was washed with ethyl acetate (50 mL) and the filtrate concentrated in vacuo. The crude material was purified by combi-flash chromatography, by eluting with 9% MeOH in DCM, to afford tert-butyl (R)-6-(4-(3-(1-(tert-butoxycarbonyl)piperidin-4-yl)propyl)benzamido)-2-(1-methylpyrrolidin-2-yl)-1H-pyrrolo[3,2-c]pyridine-1-carboxylate (Intermediate 949, 1.1 g, 59%) as a yellow solid. Mass spec m/z 646.4 [M+H]⁺.

### Step 2

### Intermediate 950: (R)—N-(2-(1-methylpyrrolidin-2-yl)-1H-pyrrolo[3,2-c]pyridin-6-yl)-4-(3-(piperidin-4-yl)propyl)benzamide dihydrochloride

To a cooled (0° C.) solution of tert-butyl (R)-6-(4-(3-(1-(tert-butoxycarbonyl)piperidin-4-yl)propyl)benzamido)-2-(1-methylpyrrolidin-2-yl)-1H-pyrrolo[3,2-c]pyridine-1-carboxylate (Intermediate 949, 1.1 g, 1.77 mmol) in 1,4-dioxane (10 mL) was added 4M hydrochloric acid in 1,4-dioxane (10 mL) and the reaction mixture was stirred at room temperature for 3 h. The reaction mixture was then concentrated in vacuo to afford (R)—N-(2-(1-methylpyrrolidin-2-yl)-1H-pyrrolo[3,2-c]pyridin-6-yl)-4-(3-(piperidin-4-yl)propyl)benzamide dihydrochloride (Intermediate 950, 1.2 g) as an off white solid, which was used for the next step without further purification. Mass spec m/z 446.4 [M+H]⁺.

### Step 3

### Intermediate 951: (R)-4-(3-(1-(3-bromo-2-formylphenyl)piperidin-4-yl)propyl)-N-(2-(1-methylpyrrolidin-2-yl)-1H-pyrrolo[3,2-c]pyridin-6-yl)benzamide

To a solution of (R)—N-(2-(1-methylpyrrolidin-2-yl)-1H-pyrrolo[3,2-c]pyridin-6-yl)-4-(3-(piperidin-4-yl)propyl)benzamide (Intermediate 950, 1.0 g, 2.24 mmol) in dimethyl sulfoxide (10 mL) was added N,N-diisopropylethylamine (1.6 mL, 9.0 mmol) followed by 2-bromo-6-fluorobenzaldehyde (CAS No. 360575-28-6, 0.6 g, 2.7 mmol) and the reaction mixture was heated at 100° C. for 16 h. The reaction mixture was quenched with water (25 mL) and extracted with ethyl acetate (2×50 mL). The combined organic layers were dried over anhydrous Na₂SO₄, filtered and concentrated in vacuo. The crude material was purified by combi-flash chromatography, by eluting with 100% ethyl acetate, to afford (R)-4-(3-(1-(3-bromo-2-formylphenyl)piperidin-4-yl)propyl)-N-(2-(1-methylpyrrolidin-2-yl)-1H-pyrrolo[3,2-c]pyridin-6-yl)benzamide (Intermediate 951, 0.90 g, 64%) as a yellow solid. ¹H NMR (400 MHz, DMSO-d₆) δ ppm 0.89-0.95 (m, 3H), 1.25-1.32 (m, 2H), 1.62-1.87 (m, 7H), 2.07-2.14 (m, 2H), 2.15 (s, 3H), 2.19-2.30 (m, 2H), 2.58-2.69 (m, 2H), 2.80-2.84 (m, 1H), 2.91-2.94 (m, 1H), 3.10-3.15 (m, 2H), 6.37 (s, 1H), 7.19-7.42 (m, 6H), 7.97 (d, J=7.83 Hz, 2H), 8.17 (s, 1H), 8.47 (s, 1H), 10.32 (br s, 1H), 11.33 (br s, 1H).

### Step 4

### Intermediate 952: tert-butyl (S)-5-amino-4-((2-bromo-6-(4-(3-(4-((2-((R)-1-methylpyrrolidin-2-yl)-1H-pyrrolo[3,2-c]pyridin-6-yl)carbamoyl)phenyl)propyl)piperidin-1-yl)benzyl)amino)-5-oxopentanoate

To a solution of (R)-4-(3-(1-(3-bromo-2-formylphenyl)piperidin-4-yl)propyl)-N-(2-(1-methylpyrrolidin-2-yl)-1H-pyrrolo[3,2-c]pyridin-6-yl)benzamide (Intermediate 951, 1.5 g, 2.4 mmol) in dimethylformamide (15 mL) were added tert-butyl (S)-4,5-diamino-5-oxopentanoate hydrochloride (CAS No. 108607-02-9, 0.57 g, 2.8 mmol) followed by triethylamine (1.0 mL, 7.14 mmol). The reaction mixture was heated at 70° C. for 2 h. Sodium cyanoborohydride (0.45 g, 7.17 mmol) was then added and the reaction mixture was heated at 70° C. for 5 h. The reaction mixture was quenched with water (300 mL), the resultant precipitate was collected by filtration and dried in vacuo to afford tert-butyl (S)-5-amino-4-((2-bromo-6-(4-(3-(4-((2-((R)-1-methylpyrrolidin-2-yl)-1H-pyrrolo[3,2-c]pyridin-6-yl)carbamoyl)phenyl)propyl)piperidin-1-yl)benzyl)amino)-5-oxopentanoate (Intermediate 952, 1.1 g, 57%) as an brown solid, which was used for the next step without further purification. Mass spec m/z 816.2 [M+H]⁺.

### Step 5

### Intermediate 953: tert-butyl (S)-5-amino-4-(4-(4-(3-(4-((2-((R)-1-methylpyrrolidin-2-yl)-1H-pyrrolo[3,2-c]pyridin-6-yl)carbamoyl)phenyl)propyl)piperidin-1-yl)-1-oxoisoindolin-2-yl)-5-oxopentanoate

To a solution tert-butyl (S)-5-amino-4-((2-bromo-6-(4-(3-(4-((2-((R)-1-methylpyrrolidin-2-yl)-1H-pyrrolo[3,2-c]pyridin-6-yl)carbamoyl)phenyl)propyl)piperidin-1-yl)benzyl)amino)-5-oxopentanoate (Intermediate 952, 0.85 g, 1.04 mmol) in 1,4-dioxane (10 mL) was added triethylamine (0.43 mL, 3.13 mmol). The reaction mixture was purged with argon for 15 min then Xantphos (0.18 g, 0.31 mmol) followed by PdCl₂(dppf) (0.04 g, 0.05 mmol) and tungsten hexacarbonyl (0.18 g, 0.52 mmol) were added. The reaction mixture was further purged with argon for 10 min and heated at 110° C. for 16 h. The reaction mixture was concentrated in vacuo. The crude material was purified by combi-flash chromatography, by eluting with 10% MeOH in DCM, to afford tert-butyl (S)-5-amino-4-(4-(4-(3-(4-((2-((R)-1-methylpyrrolidin-2-yl)-1H-pyrrolo[3,2-c]pyridin-6-yl)carbamoyl)phenyl)propyl)piperidin-1-yl)-1-oxoisoindolin-2-yl)-5-oxopentanoate (Intermediate 953, 0.54 g, 68%) as a brown solid. ¹H NMR (400 MHz, DMSO-d₆) δ ppm 1.15-1.20 (m, 10H) 1.32 (s, 9H) 1.63-1.73 (m, 2H) 1.78-1.80 (m, 2H) 1.99-2.09 (m, 2H) 2.16 (s, 3H) 2.64-2.78 (m, 4H) 3.05-3.11 (m, 4H) 3.36-3.40 (m, 2H) 4.40-4.46 (m, 1H) 4.47-4.55 (m, 1H) 4.66-4.75 (m, 1H) 7.13-7.20 (m, 2H) 7.27 (d, J=7.34 Hz, 1H) 7.35 (d, J=8.07 Hz, 2H) 7.37-7.43 (m, 1H) 7.55 (br s, 1H) 7.99 (d, J=8.07 Hz, 2H) 8.26 (s, 1H) 8.60 (s, 1H) 9.17 (s, 1H) 10.45 (br s, 2H). Mass spec m/z 763.2 [M+H]⁺.

### Step 6

### Example 232: 4-(3-(1-(2-((S)-2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide

To a solution of tert-butyl (S)-5-amino-4-(4-(4-(3-(4-((2-((R)-1-methylpyrrolidin-2-yl)-1H-pyrrolo[3,2-c]pyridin-6-yl)carbamoyl)phenyl)propyl)piperidin-1-yl)-1-oxoisoindolin-2-yl)-5-oxopentanoate (Intermediate 953, 0.1 g, 0.13 mmol) in acetic acid (10 mL) was added p-toluene sulfonic acid (0.056 g, 0.328 mmol) and the reaction mixture was heated at 80° C. for 8 h. The reaction mixture was quenched with water (20 mL) and extracted with ethyl acetate (3×100 mL). The combined organic layers were dried over anhydrous Na₂SO₄, filtered and concentrated in vacuo. The crude material was purified by chiral preparative HPLC (i-Cellulose-5 (30×250*mm, 5 m), Mobile Phase A: DCM-iPrOH (1:1), Mobile Phase B: MeCN; Eluent A:B 80:20; Flow rate: 35 mL/min) to afford 4-(3-(1-(2-((S)-2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide (Example 232, 20 mg, 22%) as a yellow solid. ¹H NMR (400 MHz, DMSO-d₆) δ ppm 1.23-1.28 (m, 2H), 1.29-1.33 (m, 3H), 1.38-1.45 (m, 1H), 1.63-1.69 (m, 2H), 1.71-1.79 (m, 3H), 1.82-1.91 (m, 2H), 1.92-2.03 (m, 2H), 2.13-2.15 (m, 1H), 2.16 (s, 3H), 2.59-2.62 (m, 1H), 2.66-2.69 (m, 1H), 2.68-2.76 (m, 4H), 2.86-2.96 (m, 1H), 3.14-3.20 (m, 1H), 3.36-3.39 (m, 2H), 4.25-4.32 (m, 1H), 4.39-4.43 (m, 1H), 5.08-5.12 (m, 1H), 6.39 (s, 1H), 7.15 (d, J=8.13 Hz, 1H), 7.29 (d, J=7.13 Hz, 1H), 7.34 (d, J=8.25 Hz, 2H), 7.39-7.45 (m, 1H), 7.99 (d, J=8.25 Hz, 2H), 8.18 (s, 1H), 8.49 (s, 1H), 10.35 (br s, 1H), 10.97 (br s, 1H), 11.36 (br s, 1H). Mass spec m/z 688.3 [M+H]⁺.

### Example 233 was Synthesised Following Scheme 225

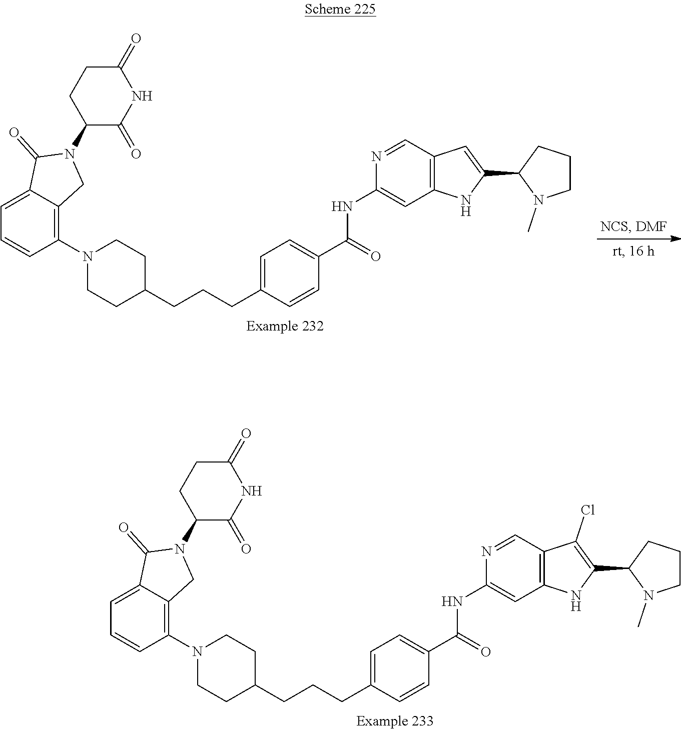 [SMILES: CN1CCC[C@@H]1c1cc2cnc(NC(=O)c3ccc(CCCC4CCN(c5cccc6c5CN([C@H]5CCC(=O)NC5=O)C6=O)CC4)cc3)cc2[nH]1 ; CN1CCC[C@@H]1c1[nH]c2cc(NC(=O)c3ccc(CCCC4CCN(c5cccc6c5CN([C@H]5CCC(=O)NC5=O)C6=O)CC4)cc3)ncc2c1Cl]

### Step 1

### Example 233: N-(3-chloro-2-((R)-1-methylpyrrolidin-2-yl)-1H-pyrrolo[3,2-c]pyridin-6-yl)-4-(3-(1-(2-((S)-2,6-dioxopiperidin-3-yl)-1-oxoisoindolin-4-yl)piperidin-4-yl)propyl)benzamide

To a solution of 4-(3-(1-(2-((S)-2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide (Example 232, 0.170 g, 0.25 mmol) in dimethylformamide (5 mL) was added N-chlorosuccinimide (0.0067 g, 0.49 mmol) and the reaction mixture was stirred at room temperature for 16 h. The reaction mixture was quenched with ice cold water (20 mL), the resultant precipitate was collected by filtration and dried in vacuo. The crude material was purified by preparative HPLC (Method A) to afford N-(3-chloro-2-((R)-1-methylpyrrolidin-2-yl)-1H-pyrrolo[3,2-c]pyridin-6-yl)-4-(3-(1-(2-((S)-2,6-dioxopiperidin-3-yl)-1-oxoisoindolin-4-yl)piperidin-4-yl)propyl)benzamide (Example 233, 0.055 g, 31%) as an off-white solid. ¹H NMR (400 MHz, DMSO-d₆) δ ppm 1.23-1.36 (m, 5H), 1.38-1.50 (m, 1H), 1.62-1.72 (m, 2H), 1.73-1.82 (m, 2H), 1.82-1.91 (m, 2H), 1.92-2.03 (m, 2H), 2.13-2.15 (m, 1H), 2.16 (s, 3H), 2.59-2.62 (m, 2H), 2.66-2.69 (m, 2H), 2.68-2.76 (m, 4H), 2.86-2.96 (m, 1H), 3.14-3.20 (m, 1H), 3.49-3.55 (m, 1H), 4.24-4.32 (m, 1H), 4.38-4.45 (m, 1H), 5.08-5.14 (m, 1H), 7.15 (d, J=8.13 Hz, 1H), 7.29 (d, J=7.13 Hz, 1H), 7.34 (d, J=8.25 Hz, 2H), 7.39-7.45 (m, 1H), 7.99 (d, J=8.25 Hz, 2H), 8.25 (s, 1H), 8.50 (s, 1H), 10.52 (br s, 1H), 10.96 (br s, 1H), 11.68 (br s, 1H). Mass spec m/z 722.3 [M+H]⁺.

### Example 234 was Synthesised Following Scheme 226

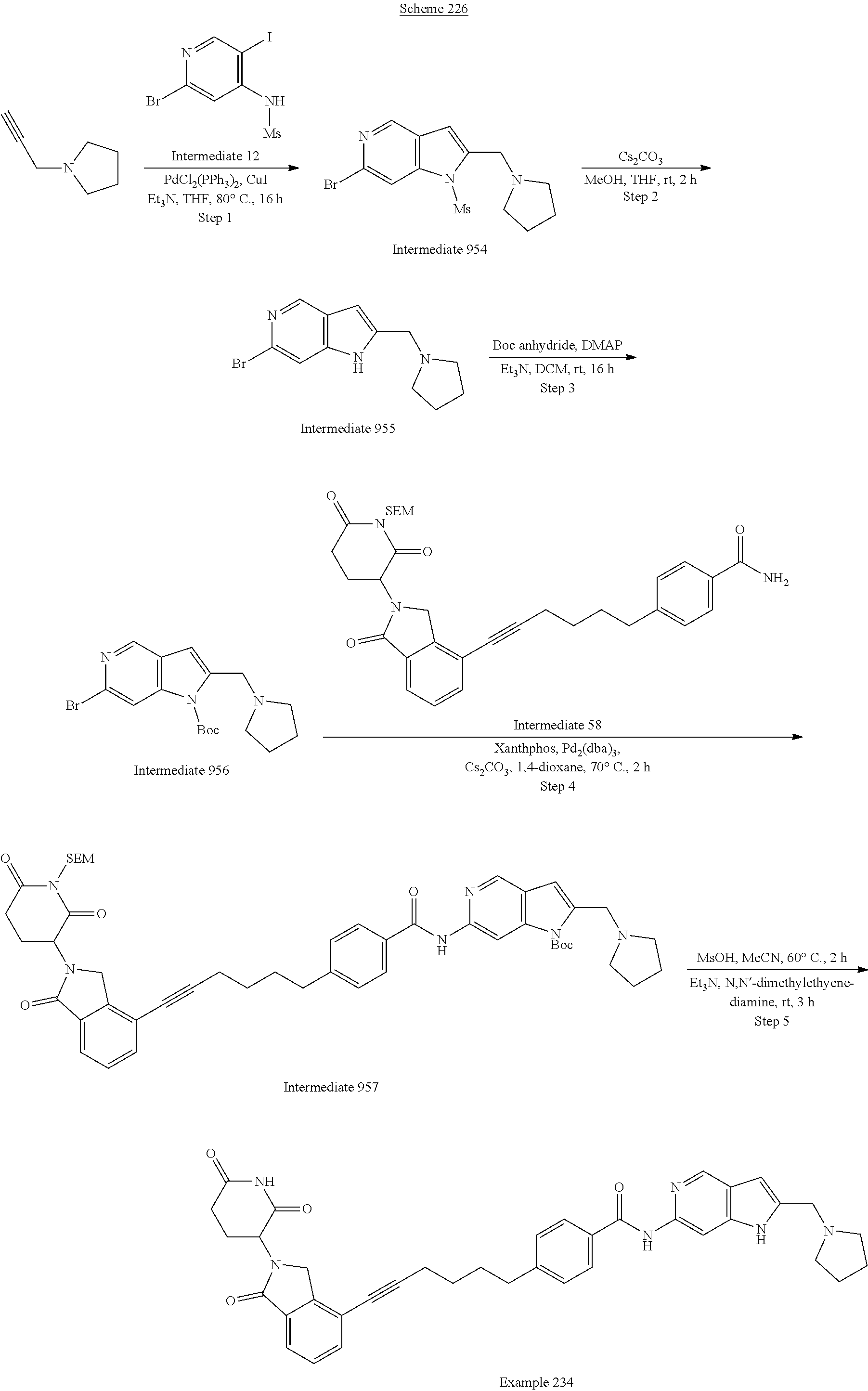

### Step 1

### Intermediate 954: 6-bromo-1-(methylsulfonyl)-2-(pyrrolidin-1-ylmethyl)-1H-pyrrolo[3,2-c]pyridine

To a solution of 1-(prop-2-yn-1-yl)pyrrolidine (CAS No. 7223-42-9, 10.0 g, 26.52 mmol) in tetrahydrofuran (100 mL) was added N-(2-bromo-5-iodopyridin-4-yl)methanesulfonamide (Intermediate 12, 4.34 g, 39.8 mmol) followed by N,N-diisopropylethylamine (37.1 mL, 212 mmol). The reaction mixture was purged with argon for 15 min then copper(I) iodide (1.01 g, 5.30 mmol) and PdCl₂(PPh₃)₂ (3.84 g, 5.30 mmol) were added. The reaction mixture was purged with argon for another 10 min and stirred at 80° C. for 16 h. The reaction mixture was concentrated in vacuo and the crude material was purified by combi-flash chromatography, by eluting with 100% ethyl acetate, to afford 6-bromo-1-(methylsulfonyl)-2-(pyrrolidin-1-ylmethyl)-1H-pyrrolo[3,2-c]pyridine (Intermediate 954, 5.8 g, 61%) as a brown solid. ¹H NMR (400 MHz, DMSO-d₆) δ ppm 1.67-1.73 (m, 4H), 2.49-2.51 (m, 4H), 3.77 (s, 3H), 3.90 (s, 2H), 6.88 (s, 1H), 7.99 (s, 1H), 8.69 (s, 1H). Mass spec m/z 359.5 [M+H]⁺.

### Step 2

### Intermediate 955: 6-bromo-2-(pyrrolidin-1-ylmethyl)-1H-pyrrolo[3,2-c]pyridine

To a cooled (0° C.) solution of 6-bromo-1-(methylsulfonyl)-2-(pyrrolidin-1-ylmethyl)-1H-pyrrolo[3,2-c]pyridine (Intermediate 954, 7.8 g, 22 mmol) in methanol (80 mL) and tetrahydrofuran (80 mL) was added cesium carbonate (11 g, 33 mmol) and the reaction mixture was stirred at room temperature for 2 h. The reaction mixture was quenched with water (2000 mL) and extracted with ethyl acetate (2×1000 mL). The combined organic layers were dried over anhydrous Na₂SO₄ and concentrated in vacuo to afford 6-bromo-2-(pyrrolidin-1-ylmethyl)-1H-pyrrolo[3,2-c]pyridine (Intermediate 955, 4.1 g) as a yellow solid, which was used for the next step without further purification. ¹H NMR (400 MHz, DMSO-d₆) δ ppm 1.68-1.74 (m, 4H), 2.43-2.48 (m, 4H), 3.72 (s, 2H), 6.43 (s, 1H), 7.44 (s, 1H), 8.50 (s, 1H), 11.63 (br s, 1H). Mass spec m/z 279.8 [M+H]⁺.

### Step 3

### Intermediate 956: tert-butyl 6-bromo-2-(pyrrolidin-1-ylmethyl)-1H-pyrrolo[3,2-c]pyridine-1-carboxylate

To a solution of 6-bromo-2-(pyrrolidin-1-ylmethyl)-1H-pyrrolo[3,2-c]pyridine (Intermediate 955, 4.1 g, 15 mmol) in dichloromethane (40 mL) was added triethylamine (6 mL, 44.0 mmol) followed by di-tert-butyl dicarbonate (6.8 mL, 29.0 mmol) and 4-(dimethylamino)pyridine (0.18 g, 1.5 mmol) and the reaction mixture was stirred at room temperature for 16 h. The reaction mixture was quenched with water (500 mL) and extracted with ethyl acetate (3×500 mL). The combined organic layers were dried over anhydrous Na₂SO₄ and concentrated in vacuo. The crude material was purified by combi-flash chromatography, eluting with ethyl acetate, to afford tert-butyl 6-bromo-2-(pyrrolidin-1-ylmethyl)-1H-pyrrolo[3,2-c]pyridine-1-carboxylate (Intermediate 956, 2.8 g, 50%) as an off-white solid. ¹H NMR (400 MHz, DMSO-d₆) δ ppm 1.63 (s, 9H), 1.68-1.74 (m, 4H), 2.51-2.56 (m, 4H), 3.92 (s, 2H), 6.77 (s, 1H), 8.02 (s, 1H), 8.62 (s, 1H). Mass spec m/z 379.9 [M+H]⁺.

### Step 4

### Intermediate 957: tert-butyl 6-(4-(6-(2-(2,6-dioxo-1-((2-(trimethylsilyl)ethoxy)methyl)piperidin-3-yl)-1-oxoisoindolin-4-yl)hex-5-yn-1-yl)benzamido)-2-(pyrrolidin-1-ylmethyl)-1H-pyrrolo[3,2-c]pyridine-1-carboxylate

To a solution of 4-(6-(2-(2,6-dioxo-1-((2-(trimethylsilyl)ethoxy)methyl)piperidin-3-yl)-1-oxoisoindolin-4-yl)hex-5-yn-1-yl)benzamide (Intermediate 58, 0.50 g, 0.87 mmol) 1,4-dioxane (10.0 mL) was added tert-butyl 6-bromo-2-(pyrrolidin-1-ylmethyl)-1H-pyrrolo[3,2-c]pyridine-1-carboxylate (Intermediate 956, 0.39 g, 1.04 mmol) followed by cesium carbonate (0.85 g, 2.61 mmol). The reaction mixture was purged with argon for 15 min then Xantphos (0.20 g, 0.34 mmol) and Pd₂(dba)₃ (0.16 g, 0.17 mmol) were added. The reaction mixture was purged with argon for another 10 min and heated at 70° C. for 2 h. The reaction mixture was concentrated in vacuo, diluted with ice cold water (50 mL) and extracted with ethyl acetate (3×50 mL). The combined organic layers were dried over anhydrous Na₂SO₄, filtered and concentrated in vacuo to afford tert-butyl 6-(4-(6-(2-(2,6-dioxo-1-((2-(trimethylsilyl)ethoxy)methyl)piperidin-3-yl)-1-oxoisoindolin-4-yl)hex-5-yn-1-yl)benzamido)-2-(pyrrolidin-1-ylmethyl)-1H-pyrrolo[3,2-c]pyridine-1-carboxylate (Intermediate 957, 0.94 g) as a brown solid, which was used for the next step without further purification. Mass spec m/z 873.4 [M+H]⁺.

### Step 5

### Example 234: 4-(6-(2-(2,6-dioxopiperidin-3-yl)-1-oxoisoindolin-4-yl)hex-5-yn-1-yl)-N-(2-(pyrrolidin-1-ylmethyl)-1H-pyrrolo[3,2-c]pyridin-6-yl)benzamide

To a solution of tert-butyl 6-(4-(6-(2-(2,6-dioxo-1-((2-(trimethylsilyl)ethoxy)methyl)piperidin-3-yl)-1-oxoisoindolin-4-yl)hex-5-yn-1-yl)benzamido)-2-(pyrrolidin-1-ylmethyl)-1H-pyrrolo[3,2-c]pyridine-1-carboxylate (Intermediate 957, 0.5 g, 0.63 mmol) in acetonitrile (10 mL) was added methanesulfonic acid (0.41 mL, 6.32 mmol) and the reaction mixture was heated at 60° C. for 2 h. After cooling to room temperature, N,N′-dimethylethylenediamine (0.34 mL, 3.16 mmol) followed by triethylamine (1.77 mL, 12.66 mmol) were added the reaction mixture was stirred at room temperature for 3 h. The reaction mixture was concentrated in vacuo, diluted with water (50 mL), the resultant precipitate was collected by filtration and dried in vacuo. The crude material was purified by preparative HPLC (Method B) to afford 4-(6-(2-(2,6-dioxopiperidin-3-yl)-1-oxoisoindolin-4-yl)hex-5-yn-1-yl)-N-(2-(pyrrolidin-1-ylmethyl)-1H-pyrrolo[3,2-c]pyridin-6-yl)benzamide (Example 234, 0.01 g, 4%) as an off-white fluffy solid. ¹H NMR (400 MHz, DMSO-d₆) δ ppm 1.60-1.64 (m, 2H), 1.69-1.76 (m, 4H), 1.76-1.81 (m, 2H), 1.98-2.05 (m, 1H), 2.38-2.49 (m, 3H), 2.52-2.60 (m, 5H), 2.71-2.75 (m, 2H), 2.86-2.97 (m, 1H), 3.71 (s, 2H), 4.28-4.33 (m, 1H), 4.42-4.47 (m, 1H), 5.12-5.16 (m, 1H), 6.36 (s, 1H), 7.35 (d, J=8.25 Hz, 2H), 7.50-7.56 (m, 1H), 7.65 (dd, J=7.63, 1.00 Hz, 1H), 7.71 (dd, J=7.63, 0.88 Hz, 1H), 7.98 (d, J=8.38 Hz, 2H), 8.19 (s, 1H), 8.51 (s, 1H), 10.35 (br s, 1H), 11.02 (br s, 1H), 11.44 (br s, 1H). Mass spec m/z 643.4 [M+H]⁺.

### Example 235: Efficacy In Vitro

### MLLT1 Degradation Assay:

MLLT1 degradation efficacy of compounds were tested in an automated chemiluminescent detection system (JESS, Cat #004-650). MV4-11 (#CRL-9591, ATCC) cells were seeded at a density of 1.2 million cells/well in a non-treated 6-well cell culture plate in 2.4 mL of complete medium (JMDM+10% FBS+1X Penicillin-Streptomycin). 10 mM DMSO stocks were prepared, and cells were treated with three concentrations of test compounds viz. 1000, 100 and 10 nM at a final DMSO concentration of 0.1% and incubated for 24 h at 37° C., 5% CO₂. Post incubation, cells were centrifuged, washed with HBSS and lysed in 70 μL of RIPA buffer supplemented with Protease inhibitors (sc-24948; Santa Cruz). Subsequently, protein estimation was done using BCA protein assay kit (23225; Pierce). 4 ug protein lysate (4 uL of 1 ug/uL lysate) was loaded into each well of the JESS cartridge (SM-W001; Bio-techne) and the JESS run was performed using 1:900 dilution of anti-human MLLT1 antibody (14893; Cell signaling technology) and 1:1200 dilution of anti-human GAPDH antibody (2118; Cell signaling technology) with anti-rabbit secondary detection module (DM-001; Bio-Techne). Post run, data were analysed using “Compass for SW” software for chemiluminescent detection. The peak area for each band was used for the calculation of MLLT1 degradation with respect to DMSO control. Data is shown as “MLLT1% degradation”.

TABLE 6

| | MLLT1% | MLLT1% | MLLT1% |
|---|---|---|---|
| Example | Degradation @ | Degradation @ | Degradation @ |
| Number | 1 μM | 0.1 μM | 0.01 μM |
| 1 | C | C | C |
| 2 | A | A | A |
| 3 | A | A | C |
| 4 | A | A | B |
| 5 | A | A | B |
| 6 | B | A | A |
| 7 | A | A | A |
| 8 | A | A | A |
| 9 | A | A | A |
| 10 | B | A | A |
| 11 | B | C | D |
| 12 | A | A | B |
| 13 | A | A | A |
| 14 | A | A | B |
| 15 | B | A | B |
| 16 | B | A | A |
| 17 | A | A | A |
| 18 | A | A | B |
| 19 | A | A | B |
| 20 | C | B | B |
| 21 | B | A | A |
| 22 | A | A | A |
| 23 | C | C | C |
| 24 | A | A | A |
| 25 | A | A | A |
| 26 | C | D | D |
| 27 | B | C | C |
| 29 | A | C | D |
| 30 | A | B | C |
| 33 | A | A | B |
| 34 | A | A | ND |
| 35 | A | A | A |
| 36 | A | A | A |
| 37 | A | A | B |
| 38 | C | D | D |
| 40 | B | C | ND |
| 41 | A | B | B |
| 42 | B | B | ND |
| 43 | B | A | ND |
| 44 | C | C | ND |
| 45 | A | A | A |
| 46 | B | B | ND |
| 47 | A | B | C |
| 49 | C | D | D |
| 50 | B | B | B |
| 51 | A | A | A |
| 52 | B | A | A |
| 53 | B | A | A |
| 54 | B | A | A |
| 55 | A | A | A |
| 56 | A | A | A |
| 57 | A | A | A |
| 58 | C | A | B |
| 59 | B | A | A |
| 60 | A | A | A |
| 61 | B | A | A |
| 62 | B | A | B |
| 63 | C | D | D |
| 64 | A | B | B |
| 65 | B | B | B |
| 66 | A | A | A |
| 67 | A | A | A |
| 69 | C | C | C |
| 71 | B | A | A |
| 72 | C | A | A |
| 73 | B | A | B |
| 74 | B | B | B |
| 75 | A | A | A |
| 76 | A | A | A |
| 77 | A | A | A |
| 78 | A | A | B |
| 79 | A | A | A |
| 80 | A | A | A |
| 81 | A | A | A |
| 82 | A | A | B |
| 83 | B | A | A |
| 84 | A | A | B |
| 85 | B | A | C |
| 86 | B | A | A |
| 87 | B | A | A |
| 88 | B | A | B |
| 89 | C | A | A |
| 91 | A | A | A |
| 92 | B | B | B |
| 93 | A | A | A |
| 94 | A | A | A |
| 95 | A | A | A |
| 96 | A | A | A |
| 97 | B | B | C |
| 98 | A | A | A |
| 99 | A | A | B |
| 100 | A | A | A |
| 101 | A | A | A |
| 102 | A | A | A |
| 103 | A | A | A |
| 106 | A | A | B |
| 107 | A | A | B |
| 108 | B | A | A |
| 109 | A | A | B |
| 110 | A | A | C |
| 111 | A | A | A |
| 112 | A | A | A |
| 113 | A | A | A |
| 114 | C | C | D |
| 115 | A | A | A |
| 116 | A | A | B |
| 117 | B | A | B |
| 118 | B | B | C |
| 119 | A | A | B |
| 120 | C | A | B |
| 121 | A | A | A |
| 122 | A | A | A |
| 123 | A | A | A |
| 124 | A | A | B |
| 125 | A | A | A |
| 126 | C | C | C |
| 127 | B | B | B |
| 128 | A | A | C |
| 129 | C | B | B |
| 130 | A | A | B |
| 131 | A | A | B |
| 132 | B | A | B |
| 133 | A | A | A |
| 134 | A | A | B |
| 135 | A | A | A |
| 136 | B | B | C |
| 137 | A | A | A |
| 138 | A | A | A |
| 139 | B | B | B |
| 140 | A | A | B |
| 141 | A | A | A |
| 142 | A | A | A |
| 143 | A | A | A |
| 144 | A | A | B |
| 145 | A | A | A |
| 146 | B | B | B |
| 147 | A | A | A |
| 148 | A | A | A |
| 149 | A | A | A |
| 150 | A | A | A |
| 151 | A | B | C |
| 152 | A | B | D |
| 153 | A | A | A |
| 154 | A | A | A |
| 155 | B | A | A |
| 156 | A | A | A |
| 157 | A | A | B |
| 158 | A | A | A |
| 159 | A | A | A |
| 160 | A | A | A |
| 161 | A | A | A |
| 162 | A | A | A |
| 163 | A | A | A |
| 164 | A | A | A |
| 165 | A | A | A |
| 166 | A | A | C |
| 167 | A | A | A |
| 168 | A | A | A |
| 169 | A | A | B |
| 170 | A | A | A |
| 171 | A | A | A |
| 172 | A | A | A |
| 173 | A | A | B |
| 174 | A | A | A |
| 175 | A | A | A |
| 176 | A | A | A |
| 177 | A | A | A |
| 178 | B | B | C |
| 179 | B | B | B |
| 180 | B | B | C |
| 181 | B | C | D |
| 182 | B | B | C |
| 183 | B | B | C |
| 184 | A | A | B |
| 185 | B | B | C |
| 186 | B | A | B |
| 187 | B | B | B |
| 188 | B | B | C |
| 189 | C | B | B |
| 190 | A | A | B |
| 191 | A | A | A |
| 192 | C | B | B |
| 193 | B | A | A |
| 194 | A | A | B |
| 195 | A | B | A |
| 196 | A | B | D |
| 197 | A | A | A |
| 198 | A | A | C |
| 199 | A | A | A |
| 200 | A | A | B |
| 201 | A | A | B |
| 202 | C | C | D |
| 203 | B | C | C |
| 204 | B | B | C |
| 205 | A | A | B |
| 206 | A | A | A |
| 207 | A | A | A |
| 208 | A | A | A |
| 209 | A | A | A |
| 210 | A | A | B |
| 211 | A | A | A |
| 212 | A | A | A |
| 213 | A | A | A |
| 214 | A | A | A |
| 215 | C | C | D |
| 216 | A | A | A |
| 217 | A | A | C |
| 218 | B | A | C |
| 219 | B | A | A |
| 220 | B | C | D |
| 221 | B | B | C |
| 222 | B | B | C |
| 223 | D | C | D |
| 224 | C | C | C |
| 225 | C | C | C |
| 226 | C | B | B |
| 227 | C | C | D |
| 228 | D | D | C |
| 229 | A | A | A |
| 230 | A | A | A |
| 231 | A | A | A |
| 233 | A | A | A |

ND = not determined
Category A: A ≥ 75% degradation
Category B: 50% ≤ B < 75% degradation
Category C: 25% ≤ C < 50% degradation
Category D: D < 25% degradation

### MV4-11 Cellular Proliferative Assays

### Assay Format A

An MV4-11 proliferation assay was used to test the ability of compounds to reduce cell viability. MV4-11 cells (ATCC #CRL9591) were grown in IMDM medium (11510596, Gibco) supplemented with 10% fetal bovine serum (F7524, Gibco) and 1% Penicillin/Streptomycin (15140-122, Gibco) at 37° C. with 5% CO₂. Cells were seeded in 384-well Elplasia plates (4447, Corning) at a density of 50 cells/well and treated with vehicle (DMSO) or compounds (10 serial semi-log dilutions, 30 μM as top concentration).

After 120 hours of treatment, cells were incubated for 10 min at RT with the CellTiter-Glo (CTG) reagent (G7571, Promega). Measurement of the luminescence signal was performed on a microplate reader (Ensight, PerkinElmer). CTG reagent measures the amount of ATP, which is directly proportional to the number of cells, to determine the number of viable cells in culture.

The data were analysed as follows:

$$Data normalization , N ( x ) = \frac{x - 〈 cr 〉}{〈 sr 〉 - 〈 cr 〉} \times 100$$

where x is the measured raw signal value of a well, (cr) is the median of the measured signal values for the Central Reference (DMSO) wells on a plate, (sr) is the median of the measured signal values for the Scale Reference (reference compound) wells on a plate.

$$Curve fitting , {S}_{0} + \frac{{S}_{inf} - {S}_{0}}{1 + {( \frac{A {C}_{50}}{{10}^{c}} )}^{n}}$$

where the experimental set-up assumes the Hill parameters in certain ranges: S₀~Central Reference (CR) from normalization (=Bottom), S_{inf}~Scale Reference (SR) from normalization (=Top), nHill~1, AC₅₀~within the measured concentration range.

### Assay Format B

MV4-11 cells at a confluency of ~50-80% was used for cell growth assay. 600 cells/well in a 384-well opaque cell culture plate (6007680; Perkin Elmer) were seeded in 40 μL of complete medium (IMDM+10% FBS+1X Penicillin-Streptomycin). Cells were incubated overnight at 37° C., 5% CO₂. 10 mM DMSO stocks were prepared, cells were treated with 10 concentrations of test compounds starting from 1 uM or 100 nM, 3-fold dilution in 10 uL (5×concentration) of complete medium. Final DMSO concentration was 0.3%. Culture plates were centrifuged at 40×g, 30s. Cells were incubated with compounds for 168 h (7 days). After 7 days, 20 uL CTG 2.0 reagent (G9242; Promega) was added per well and luminescence reading was taken in Envision Xcite 2105 multimode plate reader. Data was analyzed in GraphPad Prism 10.0 and data was shown as % viability with respect to DMSO control and dose-response curves were generated using four parameter logistic regression. Assay quality was determined by z-factor calculation using positive and negative controls.

IC₅₀ was ≤100 nM for each of the Examples set out in the Table below:

TABLE 7

| Example | |
|---|---|
| Number | Assay format |
| 2 | B |
| 3 | A |
| 4 | A |
| 6 | B |
| 7 | B |
| 8 | B |
| 9 | B |
| 10 | B |
| 12 | B |
| 13 | B |
| 14 | B |
| 15 | B |
| 16 | B |
| 17 | B |
| 19 | A |
| 20 | A |
| 21 | A |
| 22 | A |
| 24 | A |
| 25 | A |
| 29 | A |
| 33 | A |
| 34 | A |
| 35 | A |
| 36 | A |
| 37 | A |
| 40 | A |
| 41 | A |
| 42 | A |
| 43 | A |
| 45 | A |
| 47 | A |
| 48 | A |
| 51 | A |
| 52 | A |
| 53 | A |
| 54 | A |
| 55 | A |
| 56 | A |
| 57 | A |
| 58 | A |
| 59 | B |
| 60 | B |
| 61 | B |
| 62 | B |
| 65 | B |
| 66 | B |
| 67 | B |
| 68 | A |
| 75 | B |
| 76 | B |
| 77 | B |
| 78 | B |
| 79 | B |
| 80 | B |
| 81 | B |
| 82 | B |
| 83 | B |
| 85 | B |
| 86 | B |
| 87 | B |
| 88 | B |
| 89 | B |
| 91 | B |
| 93 | B |
| 94 | B |
| 95 | B |
| 96 | B |
| 98 | B |
| 99 | B |
| 100 | B |
| 101 | B |
| 102 | B |
| 103 | B |
| 104 | B |
| 105 | B |
| 107 | B |
| 108 | B |
| 109 | B |
| 111 | B |
| 112 | B |
| 113 | B |
| 115 | B |
| 116 | B |
| 117 | B |
| 118 | B |
| 119 | B |
| 120 | B |
| 121 | B |
| 122 | B |
| 123 | B |
| 124 | B |
| 125 | B |
| 129 | B |
| 131 | B |
| 132 | B |
| 133 | B |
| 134 | B |
| 135 | B |
| 137 | B |
| 138 | B |
| 140 | B |
| 141 | B |
| 142 | B |
| 143 | B |
| 144 | B |
| 145 | B |
| 147 | B |
| 148 | B |
| 149 | B |
| 150 | B |
| 153 | B |
| 154 | B |
| 155 | B |
| 156 | B |
| 157 | B |
| 158 | B |
| 159 | B |
| 160 | B |
| 161 | B |
| 162 | B |
| 163 | B |
| 164 | B |
| 165 | B |
| 167 | B |
| 168 | B |
| 169 | B |
| 170 | B |
| 171 | B |
| 172 | B |
| 173 | B |
| 174 | B |
| 175 | B |
| 176 | B |
| 177 | B |
| 186 | B |
| 190 | B |
| 191 | B |
| 193 | B |
| 194 | B |
| 195 | B |
| 197 | B |
| 199 | B |
| 206 | B |
| 207 | B |
| 209 | B |
| 210 | B |
| 211 | B |
| 212 | B |
| 213 | B |
| 216 | B |
| 230 | B |
| 231 | B |

Degradation of MLLT1 and MLLT3 by Example 55 in NIH/3T3 cells

### MLLT1 Degradation Assay:

MLLT1 degradation efficacy of compounds was tested in an automated chemiluminescent detection system (JESS, Cat #004-650). NIH/3T3 (#CRL-1658, ATCC) were seeded at a density of 0.5 million cells/well in a cell culture treated 6-well cell culture plate in 2 mL of complete medium (DMEM+10% FBS+1X Penicillin-Streptomycin) and incubated at 37° C., 5% CO₂. The next day, 10 mM DMSO stocks were prepared, and cells were treated with eight concentrations of test compound, starting from 100 nM, 5-fold dilution, at a final DMSO concentration of 0.1% and incubated for 5 h at 37° C., 5% CO₂. Post incubation, cells were washed with HBSS and lysed in 70 μL of RIPA buffer supplemented with Protease inhibitors (sc-24948; Santa Cruz). Protein estimation was done using BCA protein assay kit (23228; Pierce). 4 μg protein lysate (4 μL of 1 μg/μL lysate) was loaded into each well of the JESS cartridge (SM-W004; Bio-techne) and the JESS run was performed using 1:800 dilution of anti-mouse MLLT1 antibody (H00004298-MO1; Novus Bio) and 1:1200 dilution of anti-human GAPDH antibody (2118; Cell signalling technology) with anti-rabbit (DM-001; Bio-Techne) and anti-mouse secondary detection module (DM-002; Bio-Techne). Post run, data was analysed using “Compass for SW” software for chemiluminescent detection. Peak fit was done using the “dropped lines” method. Peak area for MLLT1 and GAPDH was used for the calculation of MLLT1 degradation with respect to DMSO control.”

### MLLT3 Degradation Assay:

MLLT3 degradation efficacy of compounds was tested in an automated chemiluminescent detection system (JESS, Cat #004-650). NIH/3T3 (#CRL-1658, ATCC) were seeded at a density of 0.5 million cells/well in a cell culture treated 6-well cell culture plate in 2 mL of complete medium (DMEM+10% FBS+IX Penicillin-Streptomycin) and incubated at 37° C., 5% CO₂. The next day, 10 mM DMSO stocks were prepared, and cells were treated with eight concentrations of test compounds, starting from 100 nM, 5-fold dilution, at a final DMSO concentration of 0.1% and incubated for 5 h at 37° C., 5% CO₂. Post incubation, cells were washed with HBSS and lysed in 70 μL of RIPA buffer supplemented with Protease inhibitors (sc-24948; Santa Cruz). Later, protein estimation was done using BCA protein assay kit (23228; Pierce). 4 μg protein lysate (4 μL of 1 μg/μL lysate) was loaded into each well of the JESS cartridge (SM-W004; Bio-techne) and the JESS run was performed using 1:200 dilution of anti-mouse MLLT3 antibody (#47577; CST) and 1:800 dilution of anti-human GAPDH antibody (2118; Cell signaling technology) with anti-rabbit secondary detection module (DM-001; Bio-Techne). Post run, data was analysed using “Compass for SW” software for chemiluminescent detection. Peak fit was done using “dropped lines” method. Peak area for MLLT3 and GAPDH was used for the calculation of MLLT3 degradation with respect to DMSO control. % Degradation for each protein was plotted against the respective doses (test compound concentration) and dose-response curve (DRC) was generated using four-parameter logistic equation (non-linear regression). The following parameters for each DRC was captured: DC₅₀ (concentration in nM at which degradation is 50%, interpolated from standard curve and transformed to linear scale), Dₘₐₓ (maximum % degradation observed). DC₅₀ and Dₘₐₓ are reported in Table 8 for Example 55.

TABLE 8

| | MLLT1 | MLLT1 | MLLT3 | MLLT3 |
|---|---|---|---|---|
| | DC₅₀ in | Dₘₐₓ in | DC₅₀ in | Dₘₐₓ in |
| | NIH/3T3 | NIH/3T3 | NIH/3T3 | NIH/3T3 |
| Example | cells (nM) | cells | cells (nM) | cells |
| 55 | 1.2 | 96% | 0.8 | 92% |

MLLT1 DC₅₀ and Dₘₐₓ Determination and Antiproliferation Cell Activity for Examples 8 and 234 in MV4-11 Cells

### MLLT1 Degradation Assay:

MLLT1 degradation efficacy of compounds was tested in an automated chemiluminescent detection system (JESS, Cat #004-650). MV4-11 (#CRL-9591, ATCC) were seeded at a density of 1.2 million cells/well in a non-treated 6-well cell culture plate in 2.4 mL of complete medium (IMDM+10% FBS+1X Penicillin-Streptomycin). 10 mM DMSO stocks were prepared, and cells were treated with eight concentrations of test compounds, starting from 100 nM, 5-fold dilution, at a final DMSO concentration of 0.1% and incubated for 4 h at 37° C., 5% CO₂. Post incubation, cells were centrifuged, washed with HBSS and lysed in 60 μL of RIPA buffer supplemented with Protease inhibitors (sc-24948; Santa Cruz). Protein estimation was done using BCA protein assay kit (23228; Pierce). 4 μg protein lysate (4 μL of 1 μg/μL lysate) was loaded into each well of the JESS cartridge (SM-W004; Bio-techne) and the JESS run was performed using 1:900 dilution of anti-human MLLT1 antibody (ABE2596; Merck) and 1:1200 dilution of anti-human GAPDH antibody (2118; Cell signaling technology) with anti-rabbit secondary detection module (DM-001; Bio-Techne). Post run, data was analysed using “Compass for SW” software for chemiluminescent detection. Peak fit was done using “dropped lines” method. Peak area for MLLT1 and GAPDH was used for the calculation of MLLT1 degradation with respect to DMSO control. Data was shown as “% degradation”. % Degradation was plotted against the respective doses (test compound concentration) and dose-response curve (DRC) was generated using four-parameter logistic equation (non-linear regression). Following parameters for each DRC was captured: DC₅₀ (concentration in nM at which degradation is 50%, interpolated from standard curve and transformed to linear scale), Dₘₐₓ (maximum % degradation observed). DC₅₀ and Dₘₐₓ data are captured in Table 9 for Example 8 and Example 234, which is a comparative example.

TABLE 9

| | | | MV4-11 | MV4-11 |
|---|---|---|---|---|
| | DC₅₀ in | Dₘₐₓ in | Antiprolif- | Antiprolif- |
| | MV4-11 | MV4-11 | eration | eration |
| Example | cells | cells | IC₅₀ (nM) | Assay Method |
| 8 | 0.4 | 87 | 1.5 | B |
| 234 | 6.4 | 60 | >1000 | B |

### Example 236: Efficacy In Vivo

In Vivo Efficacy of Example 55 from an Oral Dose

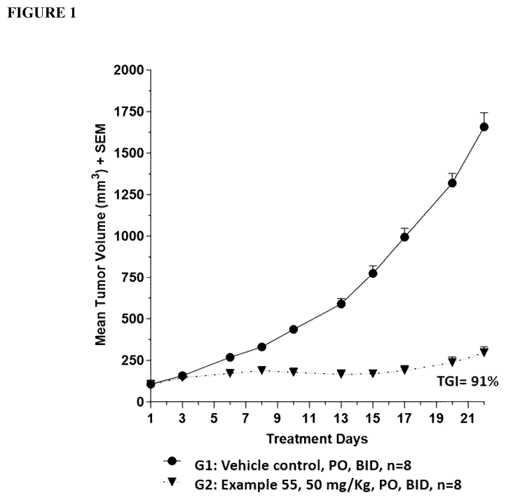

The MV4-11 subcutaneous model is an in vivo model of human AML (acute myeloid leukemia). Female NOD. SCID mice were injected subcutaneously (SC) in the right flank region with 0.2 mL (Serum free media: Matrigel; 1:1 ratio) of 5×10⁶ MV4-11 cell suspension. Tumor growth was measured by digital vernier caliper. Tumor size (volume) was measured on the day of randomization and thrice weekly until the end of the study. The tumor volume was determined by measurements of the longest axis (L) and the shortest axis (W) of tumor. Further, tumor volume was calculated by the formula: Tumor volume (mm³)=(Length×Width²)/2. Mice were randomized based on tumor volume 100-200 mm³ for efficacy evaluation. Group (G1): Vehicle Control (10 mL/kg), oral (PO), twice daily (BID); Group 2 (G2): Example 55, 50 mg/Kg, PO, BID for the indicated duration of the study. Tumor growth inhibition (TGI) calculated from tumor volume data using the following formula. TGI=(1−(Tt−T0)/(Ct−C0))×100% Tt and Ct were the mean tumor volumes of the treated and control animals on the day of treatment (measurement), and T0 and C0 were the mean tumor volumes of the treated and control animals at the start of the experiment. The TGI graph for Example 55 is shown in FIG. 1.

In one embodiment, the invention provides compounds according to the following aspects.

1. A compound which is a Proteolysis Targeting Chimera (PROTAC) or a pharmaceutically acceptable salt thereof, wherein the PROTAC has the structure:

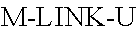

wherein U is an E3 ubiquitin ligase binding moiety, LINK is a moiety that covalently links M and U, and M is an MLLT1 and/or MLLT3 binder of formula (I):

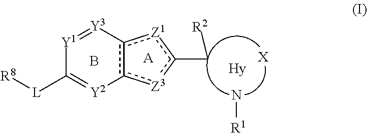

wherein:
one of Z¹ and Z³ is —N(H)— and the other is N or —C(R⁴)—, Y¹ is N, Y² is N or —C(R⁶)—, and Y³ is N or —C(R⁵)—;
Hy is a 4- to 7-membered heterocyclic ring containing X and at least one N atom, wherein: ring Hy is linked to ring A via a C atom within ring Hy, said C atom also being linked to R²; a N atom within ring Hy is substituted by R¹; X is a bond, —N(R¹¹)—, O, S, —S(O)₂—, —S(O)(NR¹¹)—, or —C(R¹¹)₂—; and the rest of ring Hy is unsubstituted or substituted by one or two R³;
L is —C(O)N(H)—, wherein the C atom of L is bonded to R⁸, and the N atom of L is bonded to ring B;
R¹ is H, C₁₋₄ cycloalkyl, or C₁₋₄ alkyl which is itself unsubstituted or substituted with one C₁₋₄ alkoxy or one, two or three halo;
R² is H or methyl;
each R³ is independently selected from C₁₋₄ alkyl, C₁₋₄ alkoxy, phenyl, a 5- to 6-membered heteroaryl ring and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring or a C₃₋₆ heterocycloalkyl ring;
R⁴ and R⁶ are independently selected from H, halo, CN, C₁₋₄ alkoxy, and C₁₋₄ alkyl which is itself unsubstituted or substituted by one, two or three halo;
R⁵ is selected from H, halo, C₁₋₄ alkoxy, C₃₋₅ cycloalkyl and C₁₋₄ alkyl which is itself unsubstituted or substituted by one, two or three halo;
R⁸ is (i) a bond to LINK or (ii) a group selected from a 6-membered aryl ring, a 5- to 6-membered heteroaryl ring, a 5- to 6-membered heterocyclyl ring, and a 5- to 6-membered cycloalkyl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo; and
each R″ is independently selected from H, C₁₋₄ alkyl, and C₁₋₄ cycloalkyl;
wherein either R⁸ is (i) a bond to LINK, or R⁸ is a group (ii) and M is bonded to LINK via a C or N atom within group R⁸ such that a hydrogen atom on the C or N atom within group R⁸ is replaced with a bond to LINK.

2. A compound according to aspect 1, wherein LINK is (a) a single bond, or (b) a chemical linker group represented by formula (L):

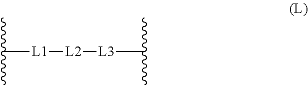

wherein:
LINK is attached to M via L1;
LINK is attached to U via L3;
L1 and L3 are each independently selected from a single bond, —N(R′)—, —C(O)N(R′)—, —O—, —N(R′)C(O)—, —C(O)—, —S(O₂)N(R′)—, —N(R′)S(O₂)—, —(C₁₋₆ alkylene)-, ethynyl, —(C₂₋₆ alkenylene)-, a divalent 3- to 6-membered cycloalkyl ring and a divalent 4- to 7-membered heterocyclyl ring;
L2 is represented by the formula -(L4)ₘ-;
each L4 is independently selected from a divalent 3- to 6-membered cycloalkyl ring, a divalent 4- to 7-membered heterocyclyl ring and a unit of formula:

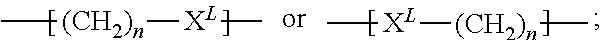

each X^{L} is independently selected from a single bond, —N(R′)—, —O—, —C(O)—, —S—, —SO—, —SO₂—, —C(H)═C(H)— and —C(H)—C(H)—;
n is selected from 1 to 4;
m is selected from 1 to 30; and
each R′ is independently selected from H and C₁₋₄ alkyl.

3. A compound according to aspect 2, wherein each L4 is independently selected from a divalent 4- to 7-membered heterocyclyl ring and a unit of formula

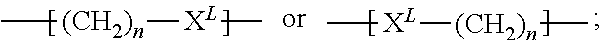

wherein each X^{L} is independently selected from a single bond, —O—, or —S—; and
n is selected from 1 or 2.

4. A compound according to aspect 2 or 3, wherein each divalent 4- to 7-membered heterocyclyl ring of L1, L2 and L3 when present contains at least one N atom.

5. A compound according to aspect 4, wherein each divalent 4- to 7-membered heterocyclyl ring of L1, L2 and L3 when present contains one or two N atoms.

6. A compound according to aspect 5, wherein each divalent 4- to 7-membered heterocyclyl ring of L1, L2 and L3 when present is independently selected from piperidinyl and diazinanyl.

7. A compound according to any one of aspects 2 to 6, wherein L2 is selected from —(CH₂)ₘ—, —(CH₂CH₂O)ₘ—, —(OCH₂CH₂)ₘ—, —(CH₂CH₂S)ₘ—, —(SCH₂CH₂)ₘ—, and a divalent 4- to 7-membered heterocyclyl ring, preferably wherein L2 is selected from —(CH₂)ₘ and a divalent 4-to 7-membered heterocyclyl ring.

8. A compound according to any one of aspects 2 to 7, wherein m is selected from 1 to 10.

9. A compound according to any one of aspects 2 to 8, wherein L1 and L3 are each independently selected from a single bond, —(C₁₋₆ alkylene)-, —N(R′)—, —C(O)N(R′)—, —O—, —N(R′)C(O)—, —C(O)—, —S(O₂)N(R′)—, —N(R′)S(O₂)—, ethynyl, and a divalent 4- to 7-membered heterocyclyl ring.

10. A compound according to any one of aspects 2 to 9, wherein L1 and L3 are each independently selected from a single bond, —(C₁₋₆ alkylene)-, —N(R′)—, —C(O)N(R′)—, —N(R′)C(O)—, —O—, ethynyl, and a divalent 4- to 7-membered heterocyclyl ring.

11. A compound according to any one of aspects 2 to 10, wherein L1 and L3 are each independently selected from a single bond, —(C₁₋₆ alkylene)-, —N(R′)—, —C(O)N(R′)—, —N(R′)C(O)—, —O—, ethynyl, piperidinyl and diazinanyl, preferably wherein L1 and L3 are each independently selected from a single bond, —(C₁₋₆ alkylene)-, —N(R′)—, —C(O)N(R′)—, —N(R′)C(O)—, ethynyl, piperidinyl and diazinanyl.

12. A compound according to any one of aspects 1 to 11, wherein U is a CRBN or VHL E3 ubiquitin ligase binding moiety.

13. A compound according to aspect 12, wherein U is a CRBN E3 ubiquitin ligase binding moiety.

14. A compound according to aspect 12, wherein U is a VHL E3 ubiquitin ligase binding moiety.

15. A compound according to any one of aspects 1 to 12, wherein U is:
(a) a CRBN E3 ubiquitin ligase binding moiety of formula (U1):

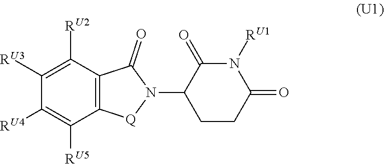

wherein:
R^{U1} is H;
Q is selected from —CH(R^{U6})—, —N(R^{U6})—, —O—, —C(O)—, —NH—CH(R^{U6})—, —N═C(R^{U6})—, or —N═N—;
R^{U6} is H or C₁₋₄alkyl;
R^{U2} and R^{U5} are each independently selected from H, halogen, C₁₋₄alkyl, NH₂, NO₂, OH, COOH, CN, CF₃, and a single bond to LINK;
R^{U3} and R^{U4} are each independently selected from H, halogen, C₁₋₄alkyl, NH₂, NO₂, OH, COOH, CN, CF₃, and a single bond to LINK;
wherein one and only one of R^{U2}, R^{U3}, R^{U4} and R^{U5} is a single bond to LINK;
(b) a CRBN E3 ubiquitin ligase binding moiety of formula (U4):

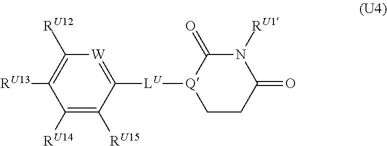

wherein:
R^{U1′} is H;
W is N or CR^{U16};
Q′ is N and L^{U} is a single bond, or Q′ is CH and L^{U} is selected from a single bond or —C(O)N(H)—, wherein either (i) the C atom of L^{U} is bonded to phenyl, and the N atom of L^{U} is bonded to Q′; or (ii) the C atom of L^{U} is bonded to Q′, and the N atom of L^{U} is bonded to phenyl;
R^{U12}, R^{U13} and R^{U14} are each independently selected from H, halogen, C₁₋₄ alkyl, NH₂, NO₂, OH, COOH, CN, CF₃, and a single bond to LINK;
wherein one and only one of R^{U2}, R^{U13} and R^{U14} is a single bond to LINK;
R^{U15} and R^{U16} are each independently selected from H, halogen, C₁₋₄ alkyl, NH₂, NO₂, OH, COOH, CN and CF₃;
(c) a CRBN E3 ubiquitin ligase binding moiety of formula (U5):

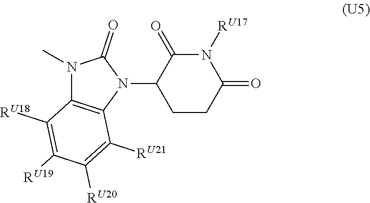

wherein:
R^{U17} is H;
R^{U18}, R^{U19}, R^{U2O} and R^{U21} are each independently selected from H, halogen, C₁₋₄ alkyl, NH₂, NO₂, OH, COOH, CN, CF₃, and a single bond to LINK;
wherein one and only one of R^{U18}, R^{U19}, R^{U20} and R^{U21} is a single bond to LINK;
or (d) a VHL E3 ubiquitin ligase binding moiety of formula (U2):

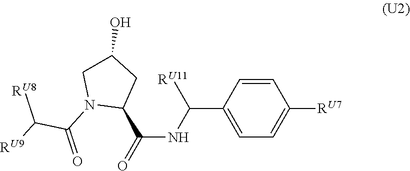

wherein:
R^{U7} is a group selected from phenyl, a 5- to 6-membered heteroaryl ring, a 5- to 6-membered heterocyclyl ring, and a 5- to 6-membered cycloalkyl ring, the group R^{U7} being unsubstituted or substituted by C₁₋₄ alkyl;
R^{U11} is H or C₁₋₄ alkyl;
R^{U8} is selected from C₁₋₄ alkyl, phenyl and a 3- to 8-membered cycloalkyl ring; and
R^{U9} is a single bond to LINK.

16. A compound according to aspect 15, wherein U is a VHL E3 ubiquitin ligase binding moiety of formula (U3):

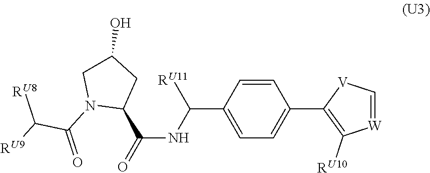

wherein:
V is S or O;
W is N or CH;
R^{U10} is H or C₁₋₄ alkyl;
R^{U11} is H or C₁₋₄ alkyl;
R^{U8} is selected from C₁₋₄ alkyl, phenyl and a 3- to 8-membered cycloalkyl ring; and
R^{U9} is a single bond to LINK.

17. A compound according to aspect 16, wherein R^{U10} is H or methyl.

18. A compound according to any one of aspects 15 to 17, wherein R^{U8} is t-butyl.

19. A compound according to aspect 15, wherein U is a CRBN ubiquitin ligase binding moiety of formula (U1), wherein R^{U6} is H or methyl.

20. A compound according to aspect 15 or 19, wherein U is a CRBN ubiquitin ligase binding moiety of formula (U1), wherein Q is selected from —C(O)— and —CH₂—.

21. A compound according to any one of aspects 15, 19 and 20, wherein U is a CRBN ubiquitin ligase binding moiety of formula (U1), wherein three of R^{U2}, R^{U3}, R^{U4} and R^{U5} are H.

22. A compound according to aspect 15, wherein U is a CRBN ubiquitin ligase binding moiety of formula (U4), wherein Q′ is N.

23. A compound according to aspect 15 or 22, wherein U is a CRBN ubiquitin ligase binding moiety of formula (U4), wherein Q′ is CH.

24. A compound according to aspect 23, wherein L^{U} is a single bond.

25. A compound according to aspect 23, wherein L^{U} is —C(O)N(H)—, wherein the C atom of L^{U} is bonded to phenyl, and the N atom of L^{U} is bonded to Q′.

26. A compound according to any one of aspects 15 and 22 to 25, wherein U is a CRBN ubiquitin ligase binding moiety of formula (U4), wherein R^{U15} and R^{U16} are H, and two of R^{U12}, R^{U13} and R^{U14} are H.

27. A compound according to any one of aspects 15 and 22 to 25, wherein U is a CRBN ubiquitin ligase binding moiety of formula (U4), wherein R^{U13}, R^{U14} and R^{U15} are H, R^{U12} is a single bond to LINK, and R^{U16} is selected from H, F and Me.

28. A compound according to aspect 15, wherein U is a CRBN ubiquitin ligase binding moiety of formula (U5), wherein three of R^{U18}, R^{U19}, R^{U20} and R^{U21} are H.

29. A compound according to any one of the preceding aspects, wherein the N atom of ring Hy which is substituted by R¹ is adjacent to the C atom of ring Hy that is bonded to ring A.

30. A compound according to any one of the preceding aspects, wherein no more than two of Y¹, Y² and Y³ are N.

31. A compound according to any one of the preceding aspects, wherein Z¹ is —C(R⁴)— and Z³ is —N(H)—.

32. A compound according to any one of the preceding aspects, wherein Z¹ is —C(R⁴)—, Z³ is —N(H)—, Y¹ is N, Y² is —C(R⁶)— and Y³ is —C(R⁵)—.

33. A compound according to any one of the preceding aspects, wherein X is a bond, —N(Me)-, O or —CH₂—.

34. A compound according to any one of the preceding aspects, wherein X is a bond.

35. A compound according to any one of the preceding aspects, wherein M is of formula (II):

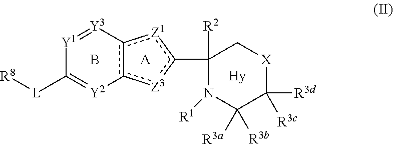

wherein:
Z¹, Z³, Y¹, Y², Y³, R¹, R², R⁸, X and L are as defined in any one of the preceding aspects;
R^{3a} and R^{3b} are independently selected from H, C₁₋₄ alkyl, phenyl, a 5- to 6-membered heteroaryl ring and C₁₋₄ alkoxy, or R^{3a} and R^{3b} form, together with the C atom to which they are attached, a C₃₋₆ cycloalkyl ring or a C₃₋₆ heterocycloalkyl ring,
R^{3c} and R^{3d} are independently selected from H, C₁₋₄ alkyl, halo, phenyl, a 5- to 6-membered heteroaryl ring and C₁₋₄ alkoxy, or R^{3c} and R^{3d} form, together with the C atom to which they are attached, a C₃₋₆ cycloalkyl ring or a C₃₋₆ heterocycloalkyl ring, or
R^{3a} and R^{3c} are H, and R^{3b} and R^{3d} form, together with the C atoms to which they are attached, a C₅₋₆ cycloalkyl ring, with the proviso that when X is —N(R¹¹)—, O, S, —S(O)₂— or —S(O)(NR¹¹)—, then neither R^{3c} nor R^{3d} are halo;
wherein at least two of R^{3a}, R^{3b}, R^{3c} and R^{3d} are H; and
wherein either (i) R⁸ is a bond to LINK, or (ii) R⁸ is a group and M is bonded to LINK via a C or N atom within group R⁸ such that a hydrogen atom on the C or N atom within group R⁸ is replaced with a bond to LINK.

36. A compound according to any one of the preceding aspects, wherein R⁴, R⁵ and R⁶ are H.

37. A compound according to any one of aspects 1 to 35, wherein one of R⁴, R⁵ and R⁶ is not H.

38. A compound according to aspect 37, wherein one of R⁴, R⁵ and R⁶ is selected from fluoro, chloro, methoxy, methyl and trifluoromethyl, and the rest are H.

39. A compound according to any one of aspects 1 to 28, wherein M is of formula (III):

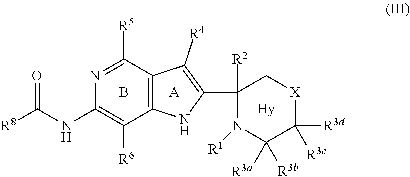

wherein:
R¹ is H or C₁₋₄ alkyl which is itself unsubstituted or substituted with one C₁₋₄ alkoxy or one, two or three halo, preferably with one C₁₋₄ alkoxy;
R² is H or methyl;
R^{3a} and R^{3b} are independently selected from H, C₁₋₄ alkyl, and C₁₋₄ alkoxy, or R^{3a} and R^{3b} form, together with the C atom to which they are attached, a C₃₋₆ cycloalkyl ring,
R^{3c} and R^{3d} are independently selected from H, C₁₋₄ alkyl, halo and C₁₋₄ alkoxy, or R^{3c} and R^{3d} form, together with the C atom to which they are attached, a C₃₋₆ cycloalkyl ring, or
R^{3a} and R^{3c} are H, and R^{3b} and R^{3d} form, together with the C atoms to which they are attached, a C₅₋₆ cycloalkyl ring,
with the proviso that when X is —N(Me)- or O, then neither R^{3c} nor R^{3d} are halo;
wherein at least two of R^{3a}, R^{3b}, R^{3c} and R^{3d} are H;
X is a bond, —N(Me)-, O or —CH₂—;
R⁴ is selected from H, CN, halo, C₁₋₄ alkoxy, and C₁₋₄ alkyl which is itself unsubstituted or substituted by one, two or three halo;
R⁵ and R⁶ are independently selected from H, halo, C₁₋₄ alkoxy, and C₁₋₄ alkyl which is itself unsubstituted or substituted by one, two or three halo;
R⁸ is (i) a bond to LINK or (ii) a group selected from a 6-membered aryl ring, a 5- to 6-membered heteroaryl ring, and a 5- to 6-membered heterocyclyl ring, the group R⁸ being unsubstituted or substituted by one, two or three substituents independently selected from halo,
C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo; and
wherein either R⁸ is (i) a bond to LINK, or R⁸ is a group (ii) and M is bonded to LINK via a C or N atom within group R⁸ such that a hydrogen atom on the C or N atom within group R⁸ is replaced with a bond to LINK.

40. A compound according to aspect 39, wherein the C or N atom within group R⁸ that is bonded to LINK is a C or N atom of the aryl, heteroaryl, or heterocyclyl, ring of group R⁸, or a C or N atom of the heteroaryl, cycloalkyl, heterocyclyl or phenyl ring of R¹⁰.

41. A compound according to any one of aspects 1 to 40, wherein R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl, more preferably pyrazolyl and cyclopropyl.

42. A compound according to aspect 41, wherein R⁸ is a 5- to 6-membered heteroaryl ring or phenyl, the group R⁸ being unsubstituted or substituted by one or two C₁₋₄ alkyl.

43. A compound according to any one of the preceding aspects, wherein Hy is an unsubstituted pyrrolidine ring, R¹ is methyl, and R² is H.

44. A compound according to aspect 1, wherein the PROTAC structure is selected from:
4-(4-(2-(2,6-dioxopiperidin-3-yl)-1-oxoisoindolin-4-yl)but-3-yn-1-yl)-N-(2-((R)-1-methylpyrrolidin-2-yl)-1H-pyrrolo[3,2-c]pyridin-6-yl)piperidine-1-carboxamide;
6-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)nicotinamide;
4-((1-(2-(2,6-dioxopiperidin-3-yl)-1,3-dioxoisoindolin-4-yl)piperidin-4-yl)methyl)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1,3-dioxoisoindolin-4-yl)piperidin-4-yl)ethyl)-N-(2-((R)-1-methylpyrrolidin-2-yl)-1H-pyrrolo[3,2-c]pyridin-6-yl)benzamide;
6-(4-((1-(2-(2,6-dioxopiperidin-3-yl)-1,3-dioxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)nicotinamide;
6-(4-(3-(2-(2,6-dioxopiperidin-3-yl)-1-oxoisoindolin-4-yl)prop-2-yn-1-yl)piperazin-1-yl)-N-(2-((R)-1-methylpyrrolidin-2-yl)-1H-pyrrolo[3,2-c]pyridin-6-yl)nicotinamide;
N—((S)-2,6-dioxopiperidin-3-yl)-6-(6-(4-((2-((R)-1-methylpyrrolidin-2-yl)-1H-pyrrolo[3,2-c]pyridin-6-yl)carbamoyl)phenyl)hex-1-yn-1-yl)picolinamide;
4-(6-(2-(2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide;
(R)-4-(6-(6-(2,4-dioxotetrahydropyrimidin-1(2H)-yl)pyridin-2-yl)hex-5-yn-1-yl)-N-(2-(1-methylpyrrolidin-2-yl)-1H-pyrrolo[3,2-c]pyridin-6-yl)benzamide;
6-(6-(2-(2,6-dioxopiperidin-3-yl)-1-oxoisoindolin-4-yl) hex-5-yn-1-yl)-N-(2-((R)-1-methylpyrrolidin-2-yl)-1H-pyrrolo[3,2-c]pyridin-6-yl) nicotinamide;
N—((S)-2,6-dioxopiperidin-3-yl)-5-(5-(4-((2-((R)-1-methylpyrrolidin-2-yl)-1H-pyrrolo[3,2-c]pyridin-6-yl)carbamoyl)phenyl)pent-1-yn-1-yl)picolinamide;
6-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)nicotinamide;
N-(3-chloro-2-((R)-1-methylpyrrolidin-2-yl)-1H-pyrrolo[3,2-c]pyridin-6-yl)-9-(2-(2,6-dioxopiperidin-3-yl)-1-oxoisoindolin-4-yl) non-8-ynamide;
3-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)benzamide;
4-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)benzamide;
6-(4-(3-(2-(2,6-dioxopiperidin-3-yl)-1-oxoisoindolin-4-yl)prop-2-yn-1-yl)piperidin-1-yl)-N-(2-((R)-1-methylpyrrolidin-2-yl)-1H-pyrrolo[3,2-c]pyridin-6-yl)nicotinamide;
6-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)nicotinamide;
6-(4-((1-(2-(2,6-dioxopiperidin-3-yl)-1,3-dioxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)nicotinamide;
4-((4-(2-(2,6-dioxopiperidin-3-yl)-1-oxoisoindolin-4-yl)but-3-yn-1-yl)oxy)-N-(2-((R)-1-methylpyrrolidin-2-yl)-1H-pyrrolo[3,2-c]pyridin-6-yl)benzamide;
4-(1-(5-(2-(2,6-dioxopiperidin-3-yl)-1-oxoisoindolin-4-yl)pent-4-yn-1-yl)-1H-pyrazol-3-yl)-N-(2-((R)-1-methylpyrrolidin-2-yl)-1H-pyrrolo[3,2-c]pyridin-6-yl)benzamide;
4-(1-(5-(2-(2,6-dioxopiperidin-3-yl)-1-oxoisoindolin-4-yl)pent-4-yn-1-yl)-1H-pyrazol-4-yl)-N-(2-((R)-1-methylpyrrolidin-2-yl)-1H-pyrrolo[3,2-c]pyridin-6-yl)benzamide;
4-(1-(5-(2-(2,6-dioxopiperidin-3-yl)-1-oxoisoindolin-5-yl)pent-4-yn-1-yl)-1H-pyrazol-4-yl)-N-(2-((R)-1-methylpyrrolidin-2-yl)-1H-pyrrolo[3,2-c]pyridin-6-yl)benzamide;
3-chloro-4-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)benzamide;
4-(((2S)-4-(2-(2,6-dioxopiperidin-3-yl)-1-oxoisoindolin-4-yl)but-3-yn-2-yl)oxy)-N-(2-((R)-1-methylpyrrolidin-2-yl)-1H-pyrrolo[3,2-c]pyridin-6-yl)benzamide;
4-(((2R)-4-(2-(2,6-dioxopiperidin-3-yl)-1-oxoisoindolin-4-yl)but-3-yn-2-yl)oxy)-N-(2-((R)-1-methylpyrrolidin-2-yl)-1H-pyrrolo[3,2-c]pyridin-6-yl)benzamide;
3-((9-(2-(2,6-dioxopiperidin-3-yl)-1,3-dioxoisoindolin-4-yl)non-8-yn-1-yl)oxy)-N-(2-((R)-1-methylpyrrolidin-2-yl)-1H-pyrrolo[3,2-c]pyridin-6-yl)benzamide;
7-{[2-(2,6-dioxopiperidin-3-yl)-1,3-dioxoisoindol-5-yl]amino}-N-{2-[(2R)-1-methylpyrrolidin-2-yl]-1H-pyrrolo[3,2-c]pyridin-6-yl}heptanamide;
7-{[2-(2,6-dioxopiperidin-3-yl)-1,3-dioxoisoindol-5-yl]amino}-N-{2-[(2S)-1-methylpyrrolidin-2-yl]-1H-pyrrolo[3,2-c]pyridin-6-yl}heptanamide;
7-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}heptanamide;
9-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}nonanamide;
12-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}dodecanamide;
12-{[2-(2,6-dioxopiperidin-3-yl)-1,3-dioxoisoindol-4-yl]amino}-N-{2-[(2S)-1-methylpyrrolidin-2-yl]-1H-pyrrolo[3,2-c]pyridin-6-yl}dodecanamide;
N-{3-chloro-2-[(2R)-1-methylpyrrolidin-2-yl]-1H-pyrrolo[3,2-c]pyridin-6-yl}-7-{[2-(2,6-dioxopiperidin-3-yl)-1,3-dioxoisoindol-4-yl]amino}heptanamide;
4-[1-(3-{1-[2-(2,6-dioxopiperidin-3-yl)-1,3-dioxoisoindol-4-yl]piperidin-4-yl}propyl)pyrazol-4-yl]-N-{2-[(2R)-1-methylpyrrolidin-2-yl]-1H-pyrrolo[3,2-c]pyridin-6-yl}benzamide;
4-[1-(3-{1-[2-(2,6-dioxopiperidin-3-yl)-1-oxo-3H-isoindol-4-yl]piperidin-4-yl}propyl)pyrazol-4-yl]-N-{2-[(2R)-1-methylpyrrolidin-2-yl]-1H-pyrrolo[3,2-c]pyridin-6-yl}benzamide;
4-(3-{[2-(2,6-dioxopiperidin-3-yl)-1,3-dioxoisoindol-4-yl]amino}propyl)-N-{2-[(2R)-1-methylpyrrolidin-2-yl]-1H-pyrrolo[3,2-c]pyridin-6-yl}benzamide;
4-(3-{[2-(2,6-dioxopiperidin-3-yl)-1,3-dioxoisoindol-4-yl]amino}propyl)-N-{2-[(2S)-1-methylpyrrolidin-2-yl]-1H-pyrrolo[3,2-c]pyridin-6-yl}benzamide;
1-(3-{1-[2-(2,6-dioxopiperidin-3-yl)-1,3-dioxoisoindol-4-yl]piperidin-4-yl}propyl)-N-{2-[(2R)-1-methylpyrrolidin-2-yl]-1H-pyrrolo[3,2-c]pyridin-6-yl}pyrazole-4-carboxamide;
2-(((1r,4R)-4-((2-(2,6-dioxopiperidin-3-yl)-1,3-dioxoisoindolin-4-yl)amino)cyclohexyl)oxy)-N-(2-((R)-1-methylpyrrolidin-2-yl)-1H-pyrrolo[3,2-c]pyridin-6-yl)acetamide;
2-(4-((2-(2,6-dioxopiperidin-3-yl)-1,3-dioxoisoindolin-4-yl)amino)butoxy)-N-(2-((R)-1-methylpyrrolidin-2-yl)-1H-pyrrolo[3,2-c]pyridin-6-yl)acetamide;
4-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)butanamide;
5-(1-(2-(2,6-Dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)pentanamide;
6-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)hexanamide;
4-(1-(2-(2,6-dioxopiperidin-3-yl)-1,3-dioxoisoindolin-4-yl)piperidin-3-yl)-N-(2-((R)-1-methylpyrrolidin-2-yl)-1H-pyrrolo[3,2-c]pyridin-6-yl)butanamide;
3-(3-((2-(2,6-dioxopiperidin-3-yl)-1,3-dioxoisoindolin-4-yl)amino)propyl)-N-(2-((R)-1-methylpyrrolidin-2-yl)-1H-pyrrolo[3,2-c]pyridin-6-yl)benzamide;
3-(((1-(2-(2,6-dioxopiperidin-3-yl)-1,3-dioxoisoindolin-4-yl)piperidin-4-yl)methyl)amino)-N-(2-((R)-1-methylpyrrolidin-2-yl)-1H-pyrrolo[3,2-c]pyridin-6-yl)benzamide;
3-(1-(3-(1-(2-(2,6-dioxopiperidin-3-yl)-1,3-dioxoisoindolin-4-yl)piperidin-4-yl)propyl)-1H-pyrazol-4-yl)-N-(2-((R)-1-methylpyrrolidin-2-yl)-1H-pyrrolo[3,2-c]pyridin-6-yl)benzamide;
4-(1-(2-(2,6-dioxopiperidin-3-yl)-1,3-dioxoisoindolin-4-yl)azetidin-3-yl)-N-(2-((R)-1-methylpyrrolidin-2-yl)-1H-pyrrolo[3,2-c]pyridin-6-yl)butanamide;
5-(4-(2-(2,6-dioxopiperidin-3-yl)-1-oxoisoindolin-4-yl)but-3-yn-1-yl)-N-(2-((R)-1-methylpyrrolidin-2-yl)-1H-pyrrolo[3,2-c]pyridin-6-yl)picolinamide;
5-[3-[2-(2,6-dioxo-3-piperidyl)-1-oxo-isoindolin-4-yl]prop-2-ynoxy]-N-[2-(1-methylpyrrolidin-2-yl)-1H-pyrrolo[3,2-c]pyridin-6-yl]pyridine-2-carboxamide;
4-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)benzamide;
4-(4-(2-(2,6-dioxopiperidin-3-yl)-1-oxoisoindolin-4-yl)but-3-yn-1-yl)-N-(2-((R)-1-methylpyrrolidin-2-yl)-1H-pyrrolo[3,2-c]pyridin-6-yl)benzamide;
4-(4-(1-(2,6-dioxopiperidin-3-yl)-3-methyl-2-oxo-2,3-dihydro-1H-benzo[d]imidazol-4-yl)but-3-yn-1-yl)-N-(2-((R)-1-methylpyrrolidin-2-yl)-1H-pyrrolo[3,2-c]pyridin-6-yl)benzamide;
4-(4-(2-(2,6-dioxopiperidin-3-yl)-1-oxoisoindolin-4-yl) but-3-yn-1-yl)-2-fluoro-N-(2-((R)-1-methylpyrrolidin-2-yl)-1H-pyrrolo[3,2-c]pyridin-6-yl)benzamide;
4-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
4-((5-(2-(2,6-dioxopiperidin-3-yl)-1-oxoisoindolin-4-yl)pent-4-yn-1-yl)oxy)-N-(2-((R)-1-methylpyrrolidin-2-yl)-1H-pyrrolo[3,2-c]pyridin-6-yl)benzamide;
4-((3-(2-(2,6-dioxopiperidin-3-yl)-1-oxoisoindolin-4-yl)prop-2-yn-1-yl)oxy)-2-fluoro-N-(2-((R)-1-methylpyrrolidin-2-yl)-1H-pyrrolo[3,2-c]pyridin-6-yl)benzamide;
3-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)benzamide;
4-(1-(6-(2-(2,6-dioxopiperidin-3-yl)-1-oxoisoindolin-4-yl)hex-5-yn-1-yl)-1H-pyrazol-4-yl)-N-(2-((R)-1-methylpyrrolidin-2-yl)-1H-pyrrolo[3,2-c]pyridin-6-yl)benzamide;
N—((S)-2,6-dioxopiperidin-3-yl)-6-(4-(3-(4-(4-((2-((R)-1-methylpyrrolidin-2-yl)-1H-pyrrolo[3,2-c]pyridin-6-yl)carbamoyl)phenyl)-1H-pyrazol-1-yl)propyl)piperidin-1-yl)picolinamide;
N—((S)-2,6-dioxopiperidin-3-yl)-6-(4-(2-(4-(4-((2-((R)-1-methylpyrrolidin-2-yl)-1H-pyrrolo[3,2-c]pyridin-6-yl)carbamoyl)phenyl)-1H-pyrazol-1-yl)ethoxy)piperidin-1-yl)picolinamide;
N-((S)-2,6-dioxopiperidin-3-yl)-6-(5-(4-(4-((2-((R)-1-methylpyrrolidin-2-yl)-1H-pyrrolo[3,2-c]pyridin-6-yl)carbamoyl)phenyl)-1H-pyrazol-1-yl)pent-1-yn-1-yl) picolinamide;
(R)-4-(5-(6-(2,4-dioxotetrahydropyrimidin-1(2H)-yl)pyridin-2-yl)pent-4-yn-1-yl)-N-(2-(1-methylpyrrolidin-2-yl)-1H-pyrrolo[3,2-c]pyridin-6-yl)benzamide;
6-(4-(4-(2-(2,6-dioxopiperidin-3-yl)-1,3-dioxoisoindolin-4-yl)piperazin-1-yl)piperidin-1-yl)-N-(2-((R)-1-methylpyrrolidin-2-yl)-1H-pyrrolo[3,2-c]pyridin-6-yl)nicotinamide;
2-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)pyrimidine-5-carboxamide;
4-((3-(1-(2,6-dioxopiperidin-3-yl)-3-methyl-2-oxo-2,3-dihydro-1H-benzo[d]imidazol-4-yl)prop-2-yn-1-yl)oxy)-N-(2-((R)-1-methylpyrrolidin-2-yl)-1H-pyrrolo[3,2-c]pyridin-6-yl)benzamide;
6-(4-(4-(2-(2,6-dioxopiperidin-3-yl)-1-oxoisoindolin-4-yl)phenyl)piperidin-1-yl)-N-(2-((R)-1-methylpyrrolidin-2-yl)-1H-pyrrolo[3,2-c]pyridin-6-yl)nicotinamide;
7-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}heptanamide;
(2S,4R)-1-[(2S)-3,3-dimethyl-2-(11-{[4-({2-[(2R)-1-methylpyrrolidin-2-yl]-1H-pyrrolo[3,2-c]pyridin-6-yl}carbamoyl)phenyl]formamido}undecanamido)butanoyl]-4-hydroxy-N-{[4-(4-methyl-1,3-thiazol-5-yl)phenyl]methyl}pyrrolidine-2-carboxamide;
(2S,4R)-1-[(2S)-3,3-dimethyl-2-(11-{[4-({2-[(2S)-1-methylpyrrolidin-2-yl]-1H-pyrrolo[3,2-c]pyridin-6-yl}carbamoyl)phenyl]formamido}undecanamido)butanoyl]-4-hydroxy-N-{[4-(4-methyl-1,3-thiazol-5-yl)phenyl]methyl}pyrrolidine-2-carboxamide;
N-(3-chloro-2-((R)-1-methylpyrrolidin-2-yl)-1H-pyrrolo[3,2-c]pyridin-6-yl)-6-(6-(2-(2,6-dioxopiperidin-3-yl)-1-oxoisoindolin-4-yl)hex-5-yn-1-yl)nicotinamide;
N-(3-chloro-2-((R)-1-methylpyrrolidin-2-yl)-1H-pyrrolo[3,2-c]pyridin-6-yl)-6-(4-(3-(2-(2,6-dioxopiperidin-3-yl)-1-oxoisoindolin-4-yl)prop-2-yn-1-yl)piperazin-1-yl)nicotinamide;
6-(4-(3-(2-(2,6-dioxopiperidin-3-yl)-1-oxoisoindolin-4-yl)prop-2-yn-1-yl)piperidin-1-yl)-N-(2-((R)-1-ethylpyrrolidin-2-yl)-1H-pyrrolo[3,2-c]pyridin-6-yl)nicotinamide;
6-(6-(2-(2,6-dioxopiperidin-3-yl)-1-oxoisoindolin-4-yl)hex-5-yn-1-yl)-N-(2-((R)-1-ethylpyrrolidin-2-yl)-1H-pyrrolo[3,2-c]pyridin-6-yl)nicotinamide; and
6-(1′-(2-(2,6-dioxopiperidin-3-yl)-1-oxoisoindolin-4-yl)-[4,4′-bipiperidin]-1-yl)-N-(2-((R)-1-methylpyrrolidin-2-yl)-1H-pyrrolo[3,2-c]pyridin-6-yl)nicotinamide.

45. A compound according to any one of aspects 1 to 28, wherein M is of formula (IV):

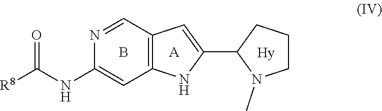

wherein R⁸ is (i) a bond to LINK or (ii) a group selected from a 6-membered aryl ring and a 5-to 6-membered heteroaryl ring, the group R⁸ being unsubstituted or substituted by one, two or three substituents independently selected from halo, C₁₋₄ alkyl and R¹⁰;
R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl, more preferably pyrazolyl and cyclopropyl;
wherein either R⁸ is (i) a bond to LINK, or R⁸ is a group (ii) and M is bonded to LINK via a C or N atom within group R⁸ such that a hydrogen atom on the C or N atom within group R⁸ is replaced with a bond to LINK.

46. A compound according to any one of aspects 1 to 45, wherein R⁸ is phenyl, pyrimidinyl or pyridinyl.

47. A compound according to any one of the preceding aspects, wherein the group R⁸ is unsubstituted or substituted by one fluoro or chloro group and/or by one group R¹⁰.

48. A compound according to any one of the preceding aspects, wherein the group R⁸ is phenyl, pyrimidinyl or pyridinyl which is unsubstituted or substituted by one fluoro or chloro group and/or by one group R¹⁰, wherein R¹⁰ is pyrazolyl, or wherein phenyl, pyrimidinyl or pyridinyl which is unsubstituted or substituted by one fluoro or chloro group.

49. A compound according to any one of aspects 45 to 48, wherein R⁸ is selected from one of the following structures:

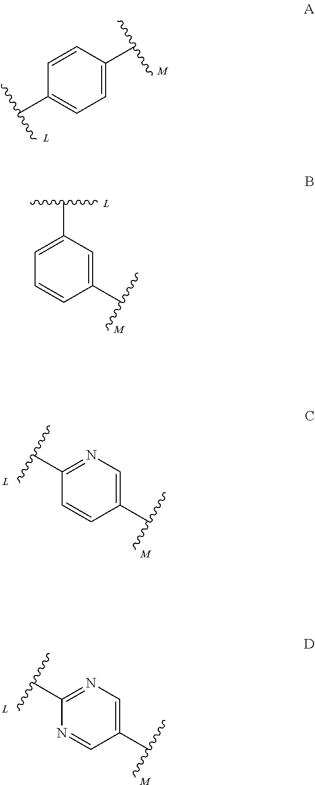

wherein:

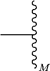

represents the point of attachment of R⁸ to the rest of moiety M;

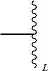

represents the point of attachment of R⁸ to LINK or R¹⁰.

50. A compound according to any one of aspects 1 to 35 and 39 to 43, wherein at least one of R⁴, R⁵ and R⁶ is not H.

51. A compound according to any one of aspects 1 to 39, wherein R⁸ is a bond to link.

52. A compound according to any one of aspects 1 to 39, wherein R⁸ is a group (ii) and M is bonded to LINK via a C or N atom within group R⁸.

53. A compound according to any one of the preceding aspects, wherein the bond from ring Hy to ring A is in the “up” position and the bond from ring Hy to R² is in the “down” position relative to ring Hy, as depicted below:

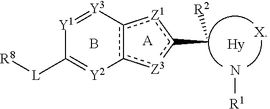

54. A pharmaceutical composition which comprises a compound as defined in any one of aspects 1 to 53 and a pharmaceutically acceptable carrier or diluent.

55. A compound as defined in any one of aspects 1 to 53 for use in the treatment of the human or animal body by therapy.

56. A compound as defined in any one of aspects 1 to 53 for use in the treatment of cancer.

57. The compound for use according to aspect 56, wherein the cancer is a transcriptionally addicted cancer, such as breast cancer, acute leukemia, prostate cancer, bladder cancer, cholangiocarcinoma, colon adenocarcinoma, esophageal carcinoma, head and neck squamous cell carcinoma, kidney chromophobe, kidney renal clear cell carcinoma, kidney renal papillary cell carcinoma, liver hepatocellular carcinoma, lung adenocarcinoma, pheochromocytoma and paraganglioma, rectum adenocarcinoma, stomach adenocarcinoma, uterine corpus endometrial carcinoma, cervical squamous cell carcinoma and endocervical adenocarcinoma, lung squamous cell carcinoma, neuroblastoma, midline glioma or sarcoma, preferably neuroblastoma or midline glioma.

58. The compound for use according to aspect 56, wherein the cancer is acute myeloid leukaemia (AML), myelodysplastic syndrome (MDS), myeloproliferative neoplasm (MPN) or acute lymphoblastic leukaemia (ALL).

59. The compound for use according to aspect 58, wherein the cancer is mixed-lineage leukaemia (MLL) or rearranged acute leukemia (AML and ALL).

60. The compound for use according to aspect 58, wherein the cancer is NPM1 mutant leukemia.

In another embodiment, the invention provides compounds according to the following aspects.

101. A compound which is a Proteolysis Targeting Chimera (PROTAC) or a pharmaceutically acceptable salt thereof, wherein the PROTAC has the structure:

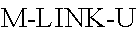

wherein U is an E3 ubiquitin ligase binding moiety, LINK is a moiety that covalently links M and U, and M is an MLLT1 and/or MLLT3 binder of formula (I):

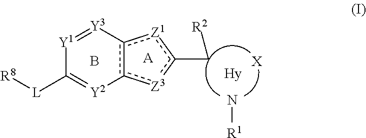

wherein:
one of Z¹ and Z³ is —N(H)— and the other is N or —C(R⁴)—, Y¹ is N, Y² is N or —C(R⁶)—, and Y³ is N or —C(R⁵)—;
Hy is a 4- to 7-membered heterocyclic ring containing X and at least one N atom, wherein: ring Hy is linked to ring A via a C atom within ring Hy, said C atom also being linked to R²; a N atom within ring Hy is substituted by R¹; X is a bond, —N(R¹¹)—, O, S, —S(O)₂—, —S(O)(NR¹¹)—, or —C(R¹¹)₂—; and the rest of ring Hy is unsubstituted or substituted by one or two R³;
L is —C(O)N(H)—, wherein the C atom of L is bonded to R⁸, and the N atom of L is bonded to ring B;
R¹ is H, C₁₋₄ cycloalkyl, or C₁₋₄ alkyl which is itself unsubstituted or substituted with one C₁₋₄ alkoxy or one, two or three halo;
R² is H or methyl;
each R³ is independently selected from C₁₋₄ alkyl, C₁₋₄ alkoxy, phenyl, a 5- to 6-membered heteroaryl ring and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring or a C₃₋₆ heterocycloalkyl ring;
R⁴ and R⁶ are independently selected from H, halo, CN, C₁₋₄ alkoxy, and C₁₋₄ alkyl which is itself unsubstituted or substituted by one, two or three halo;
R⁵ is selected from H, halo, C₁₋₄ alkoxy, C₃₋₅ cycloalkyl and C₁₋₄ alkyl which is itself unsubstituted or substituted by one, two or three halo;
R⁸ is (i) a bond to LINK or (ii) a group selected from a 6-membered aryl ring, a 5- to 6-membered heteroaryl ring, a 5- to 6-membered heterocyclyl ring, and a 5- to 6-membered cycloalkyl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo; and
each R¹¹ is independently selected from H, C₁₋₄ alkyl, and C₁₋₄ cycloalkyl;
wherein either R⁸ is (i) a bond to LINK, or R⁸ is a group (ii) and M is bonded to LINK via a C or N atom within group R⁸ such that a hydrogen atom on the C or N atom within group R⁸ is replaced with a bond to LINK.

102. A compound according to aspect 101, wherein LINK is (a) a single bond, or (b) a chemical linker group represented by formula (L):

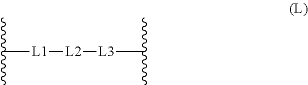

wherein:
LINK is attached to M via L1;
LINK is attached to U via L3;
L1 and L3 are each independently selected from a single bond, —N(R′)—, —C(O)N(R′)—, —O—, —N(R′)C(O)—, —C(O)—, —S(O₂)N(R′)—, —N(R′)S(O₂)—, —(C₁₋₆ alkylene)-, ethynylene, —(C₂₋₆ alkenylene)-, —C═C═C—, phenylene, a divalent 3- to 6-membered cycloalkyl ring and a divalent 4- to 7-membered heterocyclyl ring, the phenylene, cycloalkyl and heterocyclyl rings being unsubstituted or substituted by one or two C₁₋₄ alkyl;
L2 is represented by the formula -(L4)ₘ-;
each L4 is independently a unit of formula:

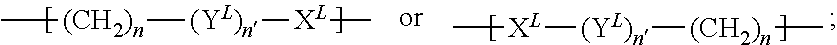

each X^{L} is independently selected from a single bond, —N(R′)—, —O—, —C(O)—, —S—, —SO—, —SO₂—, —C(R″)═C(R″)— and —C≡C—;
each Y^{L} is independently selected from a divalent 3- to 6-membered cycloalkyl ring, a divalent 4- to 7-membered heterocyclyl ring, or one of the following structures:

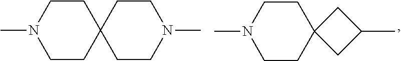

the cycloalkyl and heterocyclyl rings being unsubstituted or substituted by one or two C₁₋₄ alkyl;
n is selected from 0 to 4;
n′ is selected from 0 or 1;

$$n + {n}^{′} \geq 1 ;$$

m is selected from 1 to 30;
each R′ is independently selected from H and C₁₋₄ alkyl; and
each R″ is independently selected from H and halo.

103. A compound according to aspect 102, wherein each Y^{L} is a divalent 4- to 7-membered heterocyclyl ring, each X^{L} is independently selected from a single bond, —O—, or —S—, and n is selected from 1 or 2.

104. A compound according to aspect 102 or 103, wherein each divalent 4- to 7-membered heterocyclyl ring of L1, L2 and L3 when present contains at least one N atom.

105. A compound according to aspect 104, wherein each divalent 4- to 7-membered heterocyclyl ring of L1, L2 and L3 when present contains one or two N atoms.

106. A compound according to aspect 105, wherein each divalent 4- to 7-membered heterocyclyl ring of L1, L2 and L3 when present is independently selected from piperidinyl and diazinanyl.

107. A compound according to any one of aspects 102 to 106, wherein L2 is selected from —(CH₂)ₘ—, —(CH₂CH₂O)ₘ—, —(OCH₂CH₂)ₘ—, —(CH₂CH₂S)ₘ—, —(SCH₂CH₂)ₘ—, and a divalent 4- to 7-membered heterocyclyl ring.

108. A compound according to any one of aspects 102 to 107, wherein m is selected from 1 to 10.

109. A compound according to any one of aspects 102 to 108, wherein L1 and L3 are each independently selected from a single bond, —C(O)N(R′)—, —O—, —N(R′)C(O)—, —C(O)—, —S(O₂)N(R′)—, —N(R′)S(O₂)—, —(C₁₋₆ alkylene)-, ethynylene, —(C₂₋₆ alkenylene)-, —C═C═C—, phenylene, a divalent 3- to 6-membered cycloalkyl ring and a divalent 4- to 7-membered heterocyclyl ring, the phenylene, cycloalkyl and heterocyclyl rings being unsubstituted or substituted by one methyl group.

110. A compound according to any one of aspects 102 to 109, wherein L1 and L3 are each independently selected from a single bond, —(C₁₋₆ alkylene)-, —C(O)N(R′)—, —N(R′)C(O)—, —O—, ethynylene, and a divalent 4- to 7-membered heterocyclyl ring.

111. A compound according to any one of aspects 102 to 110, wherein L1 and L3 are each independently selected from a single bond, —(C₁₋₆ alkylene)-, —C(O)N(R′)—, —N(R′)C(O)—, —O—, ethynylene, piperidinyl and diazinanyl.

112. A compound according to any one of aspects 101 to 111, wherein U is a CRBN or VHL E3 ubiquitin ligase binding moiety.

113. A compound according to aspect 112, wherein U is a CRBN E3 ubiquitin ligase binding moiety.

114. A compound according to aspect 112, wherein U is a VHL E3 ubiquitin ligase binding moiety.

115. A compound according to any one of aspects 101 to 112, wherein U is:
(a) a CRBN E3 ubiquitin ligase binding moiety of formula (U1):

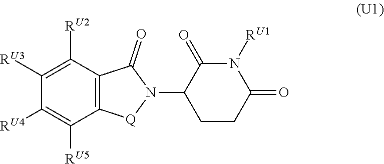

wherein:
R^{U1} is H;
Q is selected from —CH(R^{U6})—, —N(R^{U6})—, —O—, —C(O)—, —NH—CH(R^{U6})—, —N═C(R^{U6})—, or —N═N—;
R^{U6} is H or C₁₋₄alkyl;
R^{U2} and R^{U5} are each independently selected from H, halogen, C₁₋₄alkyl, NH₂, NO₂, OH, COOH, CN, CF₃, and a single bond to LINK;
R^{U3} and R^{U4} are each independently selected from H, halogen, C₁₋₄alkyl, NH₂, NO₂, OH, COOH, CN, CF₃, and a single bond to LINK;
wherein one and only one of R^{U2}, R^{U3}, R^{U4} and R^{U5} is a single bond to LINK;
(b) a CRBN E3 ubiquitin ligase binding moiety of formula (U4):

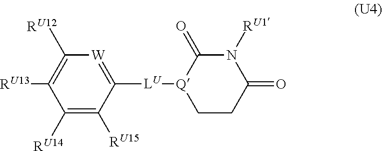

wherein:
R^{U1′} is H;
W is N or CR^{U16};
Q′ is N and L^{U} is a single bond, or Q′ is CH and L^{U} is selected from a single bond or —C(O)N(H)—, wherein either (i) the C atom of L^{U} is bonded to phenyl, and the N atom of L^{U} is bonded to Q′; or (ii) the C atom of L^{U} is bonded to Q′, and the N atom of L^{U} is bonded to phenyl;
R^{U12}, R^{U13} and R^{U14} are each independently selected from H, halogen, C₁₋₄ alkyl, NH₂, NO₂, OH, COOH, CN, CF₃, and a single bond to LINK;
wherein one and only one of R^{U12}, R^{U13} and R^{U14} is a single bond to LINK;
R^{U15} and R^{U16} are each independently selected from H, halogen, C₁₋₄ alkyl, NH₂, NO₂, OH, COOH, CN and CF₃;
(c) a CRBN E3 ubiquitin ligase binding moiety of formula (U5):

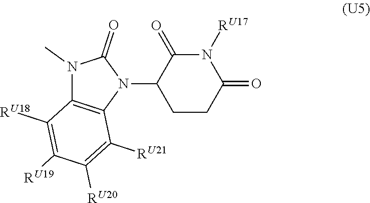

wherein:
R^{U17} is H;
R^{U18}, R^{U19}, R^{U20} and R^{U21} are each independently selected from H, halogen, C₁₋₄ alkyl, NH₂, NO₂, OH, COOH, CN, CF₃, and a single bond to LINK;
wherein one and only one of R^{U18}, R^{U19}, R^{U20} and R^{U21} is a single bond to LINK;
or (d) a VHL E3 ubiquitin ligase binding moiety of formula (U2):

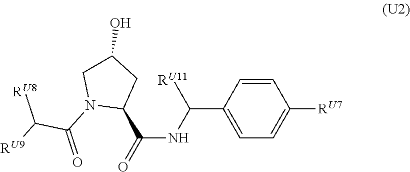

wherein:
R^{U7} is a group selected from phenyl, a 5- to 6-membered heteroaryl ring, a 5- to 6-membered heterocyclyl ring, and a 5- to 6-membered cycloalkyl ring, the group R^{U7} being unsubstituted or substituted by C₁₋₄ alkyl;
R^{U11} is H or C₁₋₄ alkyl;
R^{U8} is selected from C₁₋₄ alkyl, phenyl and a 3- to 8-membered cycloalkyl ring; and
R^{U9} is a single bond to LINK.

116. A compound according to aspect 115, wherein U is a VHL E3 ubiquitin ligase binding moiety of formula (U3):

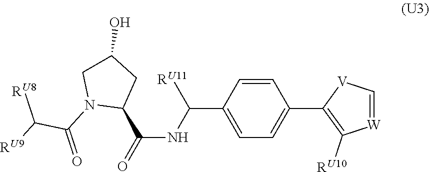

wherein:
V is S or O;
W is N or CH;
R^{U10} is H or C₁₋₄ alkyl;
R^{U11} is H or C₁₋₄ alkyl;
R^{U8} is selected from C₁₋₄ alkyl, phenyl and a 3- to 8-membered cycloalkyl ring; and
R^{U9} is a single bond to LINK.

117. A compound according to aspect 116, wherein R^{U10} is H or methyl.

118. A compound according to any one of aspects 115 to 117, wherein R^{U8} is t-butyl.

119. A compound according to aspect 115, wherein U is a CRBN ubiquitin ligase binding moiety of formula (U1), wherein R^{U6} is H or methyl.

120. A compound according to aspect 115 or 119, wherein U is a CRBN ubiquitin ligase binding moiety of formula (U1), wherein Q is selected from —C(O)— and —CH₂—.

121. A compound according to any one of aspects 115, 119 and 120, wherein U is a CRBN ubiquitin ligase binding moiety of formula (U1), wherein three of R^{U2}, R^{U3}, R^{U4} and R^{U5} are H.

122. A compound according to aspect 115, wherein U is a CRBN ubiquitin ligase binding moiety of formula (U4), wherein Q′ is N.

123. A compound according to aspect 115 or 122, wherein U is a CRBN ubiquitin ligase binding moiety of formula (U4), wherein Q′ is CH.

124. A compound according to aspect 123, wherein L^{U} is a single bond.

125. A compound according to aspect 123, wherein L^{U} is —C(O)N(H)—, wherein the C atom of L^{U} is bonded to phenyl, and the N atom of L^{U} is bonded to Q′.

126. A compound according to any one of aspects 115 and 122 to 125, wherein U is a CRBN ubiquitin ligase binding moiety of formula (U4), wherein R^{U15} and R^{U16} are H, and two of R^{U12}, R^{U13} and R^{U14} are H.

127. A compound according to any one of aspects 115 and 122 to 125, wherein U is a CRBN ubiquitin ligase binding moiety of formula (U4), wherein R^{U13}, R^{U14} and R^{U15} are H, R^{U12} is a single bond to LINK, and R^{U16} is selected from H, F and Me.

128. A compound according to aspect 115, wherein U is a CRBN ubiquitin ligase binding moiety of formula (U5), wherein three of R^{U18}, R^{U19}, R^{U20} and R^{U21} are H.

129. A compound according to any one of the preceding aspects, wherein the N atom of ring Hy which is substituted by R¹ is adjacent to the C atom of ring Hy that is bonded to ring A.

130. A compound according to any one of the preceding aspects, wherein no more than two of Y¹, Y² and Y³ are N.

131. A compound according to any one of the preceding aspects, wherein Z¹ is —C(R⁴)— and Z³ is —N(H)—.

132. A compound according to any one of the preceding aspects, wherein Z¹ is —C(R⁴)—, Z³ is —N(H)—, Y¹ is N, Y² is —C(R⁶)— and Y³ is —C(R⁵)—.

133. A compound according to any one of the preceding aspects, wherein X is a bond, —N(Me)-, O or —CH₂—.

134. A compound according to any one of the preceding aspects, wherein X is a bond.

135. A compound according to any one of the preceding aspects, wherein M is of formula (II):

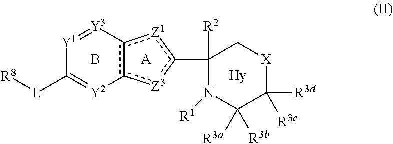

wherein:
Z₁, Z³, Y¹, Y², Y³, R¹, R², R⁸, X and L are as defined in any one of the preceding aspects;
R^{3a} and R^{3b} are independently selected from H, C₁₋₄ alkyl, phenyl, a 5- to 6-membered heteroaryl ring and C₁₋₄ alkoxy, or R^{3a} and R^{3b} form, together with the C atom to which they are attached, a C₃₋₆ cycloalkyl ring or a C₃₋₆ heterocycloalkyl ring,
R^{3c} and R^{3d} are independently selected from H, C₁₋₄ alkyl, halo, phenyl, a 5- to 6-membered heteroaryl ring and C₁₋₄ alkoxy, or R^{3c} and R^{3d} form, together with the C atom to which they are attached, a C₃₋₆ cycloalkyl ring or a C₃₋₆ heterocycloalkyl ring, or
R^{3a} and R^{3c} are H, and R^{3b} and R^{3d} form, together with the C atoms to which they are attached, a C₅₋₆ cycloalkyl ring, with the proviso that when X is —N(R¹¹)—, O, S, —S(O)₂— or —S(O)(NR¹¹)-then neither R^{3c} nor R^{3d} are halo;
wherein at least two of R^{3a}, R^{3b}, R^{3c} and R^{3d} are H; and
wherein either (i) R⁸ is a bond to LINK, or (ii) R⁸ is a group and M is bonded to LINK via a C or N atom within group R⁸ such that a hydrogen atom on the C or N atom within group R⁸ is replaced with a bond to LINK.

136. A compound according to any one of the preceding aspects, wherein R⁴, R⁵ and R⁶ are H.

137. A compound according to any one of aspects 101 to 135, wherein one of R⁴, R⁵ and R⁶ is not H.

138. A compound according to aspect 137, wherein one of R⁴, R⁵ and R⁶ is selected from fluoro, chloro, methoxy, methyl and trifluoromethyl, and the rest are H.

139. A compound according to any one of aspects 101 to 128, wherein M is of formula (III):

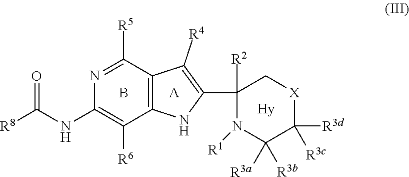

wherein:
R¹ is H or C₁₋₄ alkyl which is itself unsubstituted or substituted with one C₁₋₄ alkoxy or one, two or three halo, preferably with one C₁₋₄ alkoxy;
R² is H or methyl;
R^{3a} and R^{3b} are independently selected from H, C₁₋₄ alkyl, and C₁₋₄ alkoxy, or R^{3a} and R^{3b} form, together with the C atom to which they are attached, a C₃₋₆ cycloalkyl ring, R^{3c} and R^{3d} are independently selected from H, C₁₋₄ alkyl, halo and C₁₋₄ alkoxy, or R^{3c} and R^{3d} form, together with the C atom to which they are attached, a C₃₋₆ cycloalkyl ring, or
R^{3a} and R^{3c} are H, and R^{3b} and R^{3d} form, together with the C atoms to which they are attached, a C₅₋₆ cycloalkyl ring, with the proviso that when X is —N(Me)- or O, then neither R^{3c} nor R^{3d} are halo;
wherein at least two of R^{3a}, R^{3b}, R^{3c} and R^{3d} are H;
X is a bond, —N(Me)-, O or —CH₂—;
R⁴ is selected from H, CN, halo, C₁₋₄ alkoxy, and C₁₋₄ alkyl which is itself unsubstituted or substituted by one, two or three halo;
R⁵ and R⁶ are independently selected from H, halo, C₁₋₄ alkoxy, and C₁₋₄ alkyl which is itself unsubstituted or substituted by one, two or three halo;
R⁸ is (i) a bond to LINK or (ii) a group selected from a 6-membered aryl ring, a 5- to 6-membered heteroaryl ring, and a 5- to 6-membered heterocyclyl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo; and
wherein either R⁸ is (i) a bond to LINK, or R⁸ is a group (ii) and M is bonded to LINK via a C or N atom within group R⁸ such that a hydrogen atom on the C or N atom within group R⁸ is replaced with a bond to LINK.

140. A compound according to aspect 139, wherein the C or N atom within group R⁸ that is bonded to LINK is a C or N atom of the aryl, heteroaryl, or heterocyclyl, ring of group R⁸, or a C or N atom of the heteroaryl, cycloalkyl, heterocyclyl or phenyl ring of R¹⁰.

141. A compound according to any one of aspects 101 to 140, wherein R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl, more preferably pyrazolyl and cyclopropyl.

142. A compound according to aspect 141, wherein R⁸ is a 5- to 6-membered heteroaryl ring or phenyl, the group R⁸ being unsubstituted or substituted by one or two C₁₋₄ alkyl.

143. A compound according to any one of the preceding aspects, wherein Hy is an unsubstituted pyrrolidine ring, R^{1′} is methyl, and R² is H.

144. A compound according to aspect 101, wherein the PROTAC structure is selected from:
4-(4-(2-(2,6-dioxopiperidin-3-yl)-1-oxoisoindolin-4-yl)but-3-yn-1-yl)-N-(2-((R)-1-methylpyrrolidin-2-yl)-1H-pyrrolo[3,2-c]pyridin-6-yl)piperidine-1-carboxamide;
6-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)nicotinamide;
4-((1-(2-(2,6-dioxopiperidin-3-yl)-1,3-dioxoisoindolin-4-yl)piperidin-4-yl)methyl)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1,3-dioxoisoindolin-4-yl)piperidin-4-yl)ethyl)-N-(2-((R)-1-methylpyrrolidin-2-yl)-1H-pyrrolo[3,2-c]pyridin-6-yl)benzamide;
6-(4-((1-(2-(2,6-dioxopiperidin-3-yl)-1,3-dioxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)nicotinamide;
6-(4-(3-(2-(2,6-dioxopiperidin-3-yl)-1-oxoisoindolin-4-yl)prop-2-yn-1-yl)piperazin-1-yl)-N-(2-((R)-1-methylpyrrolidin-2-yl)-1H-pyrrolo[3,2-c]pyridin-6-yl)nicotinamide;
N—((S)-2,6-dioxopiperidin-3-yl)-6-(6-(4-((2-((R)-1-methylpyrrolidin-2-yl)-1H-pyrrolo[3,2-c]pyridin-6-yl)carbamoyl)phenyl)hex-1-yn-1-yl)picolinamide;
4-(6-(2-(2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide;
(R)-4-(6-(6-(2,4-dioxotetrahydropyrimidin-1(2H)-yl)pyridin-2-yl)hex-5-yn-1-yl)-N-(2-(1-methylpyrrolidin-2-yl)-1H-pyrrolo[3,2-c]pyridin-6-yl)benzamide;
6-(6-(2-(2,6-dioxopiperidin-3-yl)-1-oxoisoindolin-4-yl) hex-5-yn-1-yl)-N-(2-((R)-1-methylpyrrolidin-2-yl)-1H-pyrrolo[3,2-c]pyridin-6-yl) nicotinamide;
N—((S)-2,6-dioxopiperidin-3-yl)-5-(5-(4-((2-((R)-1-methylpyrrolidin-2-yl)-1H-pyrrolo[3,2-c]pyridin-6-yl)carbamoyl)phenyl)pent-1-yn-1-yl)picolinamide;
6-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)nicotinamide;
N-(3-chloro-2-((R)-1-methylpyrrolidin-2-yl)-1H-pyrrolo[3,2-c]pyridin-6-yl)-9-(2-(2,6-dioxopiperidin-3-yl)-1-oxoisoindolin-4-yl) non-8-ynamide;
3-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)benzamide;
4-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)benzamide;
6-(4-(3-(2-(2,6-dioxopiperidin-3-yl)-1-oxoisoindolin-4-yl)prop-2-yn-1-yl)piperidin-1-yl)-N-(2-((R)-1-methylpyrrolidin-2-yl)-1H-pyrrolo[3,2-c]pyridin-6-yl)nicotinamide;
6-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)nicotinamide;
6-(4-((1-(2-(2,6-dioxopiperidin-3-yl)-1,3-dioxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)nicotinamide;
4-((4-(2-(2,6-dioxopiperidin-3-yl)-1-oxoisoindolin-4-yl)but-3-yn-1-yl)oxy)-N-(2-((R)-1-methylpyrrolidin-2-yl)-1H-pyrrolo[3,2-c]pyridin-6-yl)benzamide;
4-(1-(5-(2-(2,6-dioxopiperidin-3-yl)-1-oxoisoindolin-4-yl)pent-4-yn-1-yl)-1H-pyrazol-3-yl)-N-(2-((R)-1-methylpyrrolidin-2-yl)-1H-pyrrolo[3,2-c]pyridin-6-yl)benzamide;
4-(1-(5-(2-(2,6-dioxopiperidin-3-yl)-1-oxoisoindolin-4-yl)pent-4-yn-1-yl)-1H-pyrazol-4-yl)-N-(2-((R)-1-methylpyrrolidin-2-yl)-1H-pyrrolo[3,2-c]pyridin-6-yl)benzamide;
4-(1-(5-(2-(2,6-dioxopiperidin-3-yl)-1-oxoisoindolin-5-yl)pent-4-yn-1-yl)-1H-pyrazol-4-yl)-N-(2-((R)-1-methylpyrrolidin-2-yl)-1H-pyrrolo[3,2-c]pyridin-6-yl)benzamide;
3-chloro-4-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)benzamide;
4-(((2S)-4-(2-(2,6-dioxopiperidin-3-yl)-1-oxoisoindolin-4-yl)but-3-yn-2-yl)oxy)-N-(2-((R)-1-methylpyrrolidin-2-yl)-1H-pyrrolo[3,2-c]pyridin-6-yl)benzamide;
4-(((2R)-4-(2-(2,6-dioxopiperidin-3-yl)-1-oxoisoindolin-4-yl)but-3-yn-2-yl)oxy)-N-(2-((R)-1-methylpyrrolidin-2-yl)-1H-pyrrolo[3,2-c]pyridin-6-yl)benzamide;
3-((9-(2-(2,6-dioxopiperidin-3-yl)-1,3-dioxoisoindolin-4-yl)non-8-yn-1-yl)oxy)-N-(2-((R)-1-methylpyrrolidin-2-yl)-1H-pyrrolo[3,2-c]pyridin-6-yl)benzamide;
7-{[2-(2,6-dioxopiperidin-3-yl)-1,3-dioxoisoindol-5-yl]amino}-N-{2-[(2R)-1-methylpyrrolidin-2-yl]-1H-pyrrolo[3,2-c]pyridin-6-yl}heptanamide;
7-{[2-(2,6-dioxopiperidin-3-yl)-1,3-dioxoisoindol-5-yl]amino}-N-{2-[(2S)-1-methylpyrrolidin-2-yl]-1H-pyrrolo[3,2-c]pyridin-6-yl}heptanamide;
7-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}heptanamide;
9-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}nonanamide;
12-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}dodecanamide;
12-{[2-(2,6-dioxopiperidin-3-yl)-1,3-dioxoisoindol-4-yl]amino}-N-{2-[(2S)-1-methylpyrrolidin-2-yl]-1H-pyrrolo[3,2-c]pyridin-6-yl}dodecanamide;
N-{3-chloro-2-[(2R)-1-methylpyrrolidin-2-yl]-1H-pyrrolo[3,2-c]pyridin-6-yl}-7-{[2-(2,6-dioxopiperidin-3-yl)-1,3-dioxoisoindol-4-yl]amino}heptanamide;
4-[1-(3-{1-[2-(2,6-dioxopiperidin-3-yl)-1,3-dioxoisoindol-4-yl]piperidin-4-yl}propyl)pyrazol-4-yl]-N-{2-[(2R)-1-methylpyrrolidin-2-yl]-1H-pyrrolo[3,2-c]pyridin-6-yl}benzamide;
4-[1-(3-{1-[2-(2,6-dioxopiperidin-3-yl)-1-oxo-3H-isoindol-4-yl]piperidin-4-yl}propyl)pyrazol-4-yl]-N-{2-[(2R)-1-methylpyrrolidin-2-yl]-1H-pyrrolo[3,2-c]pyridin-6-yl}benzamide;
4-(3-{[2-(2,6-dioxopiperidin-3-yl)-1,3-dioxoisoindol-4-yl]amino}propyl)-N-{2-[(2R)-1-methylpyrrolidin-2-yl]-1H-pyrrolo[3,2-c]pyridin-6-yl}benzamide;
4-(3-{[2-(2,6-dioxopiperidin-3-yl)-1,3-dioxoisoindol-4-yl]amino}propyl)-N-{2-[(2S)-1-methylpyrrolidin-2-yl]-1H-pyrrolo[3,2-c]pyridin-6-yl}benzamide;
1-(3-{1-[2-(2,6-dioxopiperidin-3-yl)-1,3-dioxoisoindol-4-yl]piperidin-4-yl}propyl)-N-{2-[(2R)-1-methylpyrrolidin-2-yl]-1H-pyrrolo[3,2-c]pyridin-6-yl}pyrazole-4-carboxamide;
2-(((1r,4R)-4-((2-(2,6-dioxopiperidin-3-yl)-1,3-dioxoisoindolin-4-yl)amino)cyclohexyl)oxy)-N-(2-((R)-1-methylpyrrolidin-2-yl)-1H-pyrrolo[3,2-c]pyridin-6-yl)acetamide;
2-(4-((2-(2,6-dioxopiperidin-3-yl)-1,3-dioxoisoindolin-4-yl)amino)butoxy)-N-(2-((R)-1-methylpyrrolidin-2-yl)-1H-pyrrolo[3,2-c]pyridin-6-yl)acetamide;
4-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)butanamide;
5-(1-(2-(2,6-Dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)pentanamide;
6-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)hexanamide;
4-(1-(2-(2,6-dioxopiperidin-3-yl)-1,3-dioxoisoindolin-4-yl)piperidin-3-yl)-N-(2-((R)-1-methylpyrrolidin-2-yl)-1H-pyrrolo[3,2-c]pyridin-6-yl)butanamide;
3-(3-((2-(2,6-dioxopiperidin-3-yl)-1,3-dioxoisoindolin-4-yl)amino)propyl)-N-(2-((R)-1-methylpyrrolidin-2-yl)-1H-pyrrolo[3,2-c]pyridin-6-yl)benzamide;
3-(((1-(2-(2,6-dioxopiperidin-3-yl)-1,3-dioxoisoindolin-4-yl)piperidin-4-yl)methyl)amino)-N-(2-((R)-1-methylpyrrolidin-2-yl)-1H-pyrrolo[3,2-c]pyridin-6-yl)benzamide;
3-(1-(3-(1-(2-(2,6-dioxopiperidin-3-yl)-1,3-dioxoisoindolin-4-yl)piperidin-4-yl)propyl)-1H-pyrazol-4-yl)-N-(2-((R)-1-methylpyrrolidin-2-yl)-1H-pyrrolo[3,2-c]pyridin-6-yl)benzamide;
4-(1-(2-(2,6-dioxopiperidin-3-yl)-1,3-dioxoisoindolin-4-yl)azetidin-3-yl)-N-(2-((R)-1-methylpyrrolidin-2-yl)-1H-pyrrolo[3,2-c]pyridin-6-yl)butanamide;
5-(4-(2-(2,6-dioxopiperidin-3-yl)-1-oxoisoindolin-4-yl)but-3-yn-1-yl)-N-(2-((R)-1-methylpyrrolidin-2-yl)-1H-pyrrolo[3,2-c]pyridin-6-yl)picolinamide;
5-[3-[2-(2,6-dioxo-3-piperidyl)-1-oxo-isoindolin-4-yl]prop-2-ynoxy]-N-[2-(1-methylpyrrolidin-2-yl)-1H-pyrrolo[3,2-c]pyridin-6-yl]pyridine-2-carboxamide;
4-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)benzamide;
4-(4-(2-(2,6-dioxopiperidin-3-yl)-1-oxoisoindolin-4-yl)but-3-yn-1-yl)-N-(2-((R)-1-methylpyrrolidin-2-yl)-1H-pyrrolo[3,2-c]pyridin-6-yl)benzamide;
4-(4-(1-(2,6-dioxopiperidin-3-yl)-3-methyl-2-oxo-2,3-dihydro-1H-benzo[d]imidazol-4-yl)but-3-yn-1-yl)-N-(2-((R)-1-methylpyrrolidin-2-yl)-1H-pyrrolo[3,2-c]pyridin-6-yl)benzamide;
4-(4-(2-(2,6-dioxopiperidin-3-yl)-1-oxoisoindolin-4-yl) but-3-yn-1-yl)-2-fluoro-N-(2-((R)-1-methylpyrrolidin-2-yl)-1H-pyrrolo[3,2-c]pyridin-6-yl)benzamide;
4-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
4-((5-(2-(2,6-dioxopiperidin-3-yl)-1-oxoisoindolin-4-yl)pent-4-yn-1-yl)oxy)-N-(2-((R)-1-methylpyrrolidin-2-yl)-1H-pyrrolo[3,2-c]pyridin-6-yl)benzamide;
4-((3-(2-(2,6-dioxopiperidin-3-yl)-1-oxoisoindolin-4-yl)prop-2-yn-1-yl)oxy)-2-fluoro-N-(2-((R)-1-methylpyrrolidin-2-yl)-1H-pyrrolo[3,2-c]pyridin-6-yl)benzamide;
3-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)benzamide;
4-(1-(6-(2-(2,6-dioxopiperidin-3-yl)-1-oxoisoindolin-4-yl)hex-5-yn-1-yl)-1H-pyrazol-4-yl)-N-(2-((R)-1-methylpyrrolidin-2-yl)-1H-pyrrolo[3,2-c]pyridin-6-yl)benzamide;
N—((S)-2,6-dioxopiperidin-3-yl)-6-(4-(3-(4-(4-((2-((R)-1-methylpyrrolidin-2-yl)-1H-pyrrolo[3,2-c]pyridin-6-yl)carbamoyl)phenyl)-1H-pyrazol-1-yl)propyl)piperidin-1-yl)picolinamide;
N—((S)-2,6-dioxopiperidin-3-yl)-6-(4-(2-(4-(4-((2-((R)-1-methylpyrrolidin-2-yl)-1H-pyrrolo[3,2-c]pyridin-6-yl)carbamoyl)phenyl)-1H-pyrazol-1-yl)ethoxy)piperidin-1-yl)picolinamide;
N—((S)-2,6-dioxopiperidin-3-yl)-6-(5-(4-(4-((2-((R)-1-methylpyrrolidin-2-yl)-1H-pyrrolo[3,2-c]pyridin-6-yl)carbamoyl)phenyl)-1H-pyrazol-1-yl)pent-1-yn-1-yl) picolinamide;
(R)-4-(5-(6-(2,4-dioxotetrahydropyrimidin-1(2H)-yl)pyridin-2-yl)pent-4-yn-1-yl)-N-(2-(1-methylpyrrolidin-2-yl)-1H-pyrrolo[3,2-c]pyridin-6-yl)benzamide;
6-(4-(4-(2-(2,6-dioxopiperidin-3-yl)-1,3-dioxoisoindolin-4-yl)piperazin-1-yl)piperidin-1-yl)-N-(2-((R)-1-methylpyrrolidin-2-yl)-1H-pyrrolo[3,2-c]pyridin-6-yl)nicotinamide;
2-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)pyrimidine-5-carboxamide;
4-((3-(1-(2,6-dioxopiperidin-3-yl)-3-methyl-2-oxo-2,3-dihydro-1H-benzo[d]imidazol-4-yl)prop-2-yn-1-yl)oxy)-N-(2-((R)-1-methylpyrrolidin-2-yl)-1H-pyrrolo[3,2-c]pyridin-6-yl)benzamide;
6-(4-(4-(2-(2,6-dioxopiperidin-3-yl)-1-oxoisoindolin-4-yl)phenyl)piperidin-1-yl)-N-(2-((R)-1-methylpyrrolidin-2-yl)-1H-pyrrolo[3,2-c]pyridin-6-yl)nicotinamide;
7-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}heptanamide;
(2S,4R)-1-[(2S)-3,3-dimethyl-2-(11-{[4-({2-[(2R)-1-methylpyrrolidin-2-yl]-1H-pyrrolo[3,2-c]pyridin-6-yl}carbamoyl)phenyl]formamido}undecanamido)butanoyl]-4-hydroxy-N-{[4-(4-methyl-1,3-thiazol-5-yl)phenyl]methyl}pyrrolidine-2-carboxamide;
(2S,4R)-1-[(2S)-3,3-dimethyl-2-(11-{[4-({2-[(2S)-1-methylpyrrolidin-2-yl]-1H-pyrrolo[3,2-c]pyridin-6-yl}carbamoyl)phenyl]formamido}undecanamido)butanoyl]-4-hydroxy-N-{[4-(4-methyl-1,3-thiazol-5-yl)phenyl]methyl}pyrrolidine-2-carboxamide;
N-(3-chloro-2-((R)-1-methylpyrrolidin-2-yl)-1H-pyrrolo[3,2-c]pyridin-6-yl)-6-(6-(2-(2,6-dioxopiperidin-3-yl)-1-oxoisoindolin-4-yl)hex-5-yn-1-yl)nicotinamide;
N-(3-chloro-2-((R)-1-methylpyrrolidin-2-yl)-1H-pyrrolo[3,2-c]pyridin-6-yl)-6-(4-(3-(2-(2,6-dioxopiperidin-3-yl)-1-oxoisoindolin-4-yl)prop-2-yn-1-yl)piperazin-1-yl)nicotinamide;
6-(4-(3-(2-(2,6-dioxopiperidin-3-yl)-1-oxoisoindolin-4-yl)prop-2-yn-1-yl)piperidin-1-yl)-N-(2-((R)-1-ethylpyrrolidin-2-yl)-1H-pyrrolo[3,2-c]pyridin-6-yl)nicotinamide;
6-(6-(2-(2,6-dioxopiperidin-3-yl)-1-oxoisoindolin-4-yl)hex-5-yn-1-yl)-N-(2-((R)-1-ethylpyrrolidin-2-yl)-1H-pyrrolo[3,2-c]pyridin-6-yl)nicotinamide;
6-(1′-(2-(2,6-dioxopiperidin-3-yl)-1-oxoisoindolin-4-yl)-[4,4′-bipiperidin]-1-yl)-N-(2-((R)-1-methylpyrrolidin-2-yl)-1H-pyrrolo[3,2-c]pyridin-6-yl)nicotinamide;
4-(3-(1-(2-(2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide;
4-(3-(1-(2-((rel-S)-2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide;
4-(3-(1-(2-((rel-R)-2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide
4-(2-(1-(2-((rel-S)-2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide;
4-(2-(1-(2-((rel-R)-2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1,3-dioxoisoindolin-4-yl) piperidin-4-yl) ethoxy)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1,3-dioxoisoindolin-5-yl)piperidin-4-yl)ethoxy)-N-(2-((R)-1-methylpyrrolidin-2-yl)-1H-pyrrolo[3,2-c]pyridin-6-yl)benzamide;
4-(5-(2-(2,6-dioxopiperidin-3-yl)-1-oxoisoindolin-4-yl)pent-4-yn-1-yl)-3,5-difluoro-N-(2-((R)-1-methylpyrrolidin-2-yl)-1H-pyrrolo[3,2-c]pyridin-6-yl benzamide;
4-(4-((4-(2-(2,6-dioxopiperidin-3-yl)-1-oxoisoindolin-4-yl)benzyl)oxy)piperidin-1-yl)-N-(2-((R)-1-methylpyrrolidin-2-yl)-1H-pyrrolo[3,2-c]pyridin-6-yl)benzamide;
6-(6-(2-(2,6-dioxopiperidin-3-yl)-1-oxoisoindolin-4-yl)hex-5-yn-1-yl)-N-(2-((rel-R)-5-methyl-5-azaspiro[2.4]heptan-6-yl)-1H-pyrrolo[3,2-c]pyridin-6-yl)nicotinamide;
4-((4-(2-(2,6-dioxopiperidin-3-yl)-1-oxoisoindolin-4-yl)but-3-yn-1-yl) (methyl)amino)-N-(2-((R)-1-methylpyrrolidin-2-yl)-1H-pyrrolo[3,2-c]pyridin-6-yl)benzamide;
4-(4-(2-(1-(2-(2,6-dioxopiperidin-3-yl)-1-oxoisoindolin-4-yl)piperidin-4-yl)acetyl) piperazin-1-yl)-N-(2-((R)-1-methylpyrrolidin-2-yl)-1H-pyrrolo[3,2-c]pyridin-6-yl)benzamide;
2-(4-(3-(2-(2,6-dioxopiperidin-3-yl)-1-oxoisoindolin-4-yl)prop-2-yn-1-yl)piperazin-1-yl)-N-(2-((R)-1-methylpyrrolidin-2-yl)-1H-pyrrolo[3,2-c]pyridin-6-yl)pyrimidine-5-carboxamide;
4-(5-(2-(2,6-dioxopiperidin-3-yl)-7-fluoro-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
4-(4-((4-(2-(2,6-dioxopiperidin-3-yl)-1-oxoisoindolin-4-yl)piperazin-1-yl)methyl)piperidin-1-yl)-N-(2-((R)-1-methylpyrrolidin-2-yl)-1H-pyrrolo[3,2-c]pyridin-6-yl)benzamide;
4-(5-(2-(2,6-dioxopiperidin-3-yl)-1-oxoisoindolin-4-yl)pent-4-yn-1-yl)-3-fluoro-N-(2-((R)-1-methylpyrrolidin-2-yl)-1H-pyrrolo[3,2-c]pyridin-6-yl)benzamide;
4-(5-(2-(2,6-dioxopiperidin-3-yl)-1-oxoisoindolin-4-yl)pent-4-yn-1-yl)-2-fluoro-N-(2-((R)-1-methylpyrrolidin-2-yl)-1H-pyrrolo[3,2-c]pyridin-6-yl)benzamide;
3-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
3-(6-(2-(2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide;
N—((S)-2,6-dioxopiperidin-3-yl)-6-(5-(4-((2-((R)-1-methylpyrrolidin-2-yl)-1H-pyrrolo[3,2-c]pyridin-6-yl)carbamoyl)phenyl)pent-1-yn-1-yl)picolinamide;
4-(4-(4-(2-(2,6-dioxopiperidin-3-yl)-1-oxoisoindolin-4-yl)benzyl)piperazin-1-yl)-N-(2-((R)-1-methylpyrrolidin-2-yl)-1H-pyrrolo[3,2-c]pyridin-6-yl)benzamide;
6-(4-(4-(2-(2,6-dioxopiperidin-3-yl)-1-oxoisoindolin-4-yl)-3-methylphenyl)piperidin-1-yl)-N-(2-((R)-1-methylpyrrolidin-2-yl)-1H-pyrrolo[3,2-c]pyridin-6-yl)nicotinamide;
N-(3-chloro-2-((R)-1-methylpyrrolidin-2-yl)-1H-pyrrolo[3,2-c]pyridin-6-yl)-4-(5-(2-(2,6-dioxopiperidin-3-yl)-1-oxoisoindolin-4-yl)pent-4-yn-1-yl)benzamide;
4-(5-(2-(2,6-dioxopiperidin-3-yl)-6-fluoro-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
4-(5-(2-((rel-S)-2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
4-(5-(2-((rel-R)-2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
4-(6-(2-((rel-S)-2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide;
4-(6-(2-((rel-R)-2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide;
4-(5-(2-(2,6-dioxopiperidin-3-yl)-6-fluoro-1-oxoisoindolin-5-yl)pent-4-yn-1-yl)-N-(2-((R)-1-methylpyrrolidin-2-yl)-1H-pyrrolo[3,2-c]pyridin-6-yl)benzamide;
4-(1-(2-(1-(2-(2,6-dioxopiperidin-3-yl)-1-oxoisoindolin-4-yl)piperidin-4-yl)acetyl)piperidin-4-yl)-N-(2-((R)-1-methylpyrrolidin-2-yl)-1H-pyrrolo[3,2-c]pyridin-6-yl)benzamide;
6-(4-(2-(1-(2-(2,6-dioxopiperidin-3-yl)-1-oxoisoindolin-4-yl)piperidin-4-yl)acetyl)piperazin-1-yl)-N-(2-((R)-1-methylpyrrolidin-2-yl)-1H-pyrrolo[3,2-c]pyridin-6-yl)nicotinamide;
6-(4-((4-(2-(2,6-dioxopiperidin-3-yl)-1-oxoisoindolin-4-yl)benzyl)oxy)piperidin-1-yl)-N-(2-((R)-1-methylpyrrolidin-2-yl)-1H-pyrrolo[3,2-c]pyridin-6-yl)nicotinamide;
4-(5-(2-(2,6-dioxopiperidin-3-yl)-1,3-dioxoisoindolin-4-yl)pent-4-yn-1-yl)-N-(2-((R)-1-methylpyrrolidin-2-yl)-1H-pyrrolo[3,2-c]pyridin-6-yl)benzamide;
4-(5-(2-(2,6-dioxopiperidin-3-yl)-1-oxoisoindolin-4-yl)pent-4-yn-1-yl)-N-(2-((R)-1-(methyl-d3)pyrrolidin-2-yl)-1H-pyrrolo[3,2-c]pyridin-6-yl)benzamide;
4-(6-(2-(2,6-dioxopiperidin-3-yl)-1-oxoisoindolin-4-yl)hex-5-yn-1-yl)-2-fluoro-N-(2-((R)-1-methylpyrrolidin-2-yl)-1H-pyrrolo[3,2-c]pyridin-6-yl)benzamide;
N-(3-chloro-2-((R)-1-methylpyrrolidin-2-yl)-1H-pyrrolo[3,2-c]pyridin-6-yl)-4-(6-(2-(2,6-dioxopiperidin-3-yl)-1-oxoisoindolin-4-yl)hex-5-yn-1-yl)-2-fluorobenzamide;
4-(6-(2-(2,6-dioxopiperidin-3-yl)-6-fluoro-1-oxoisoindolin-5-yl)hex-5-yn-1-yl)-2-fluoro-N-(2-((R)-1-methylpyrrolidin-2-yl)-1H-pyrrolo[3,2-c]pyridin-6-yl)benzamide;
4-(6-(2-(2,6-dioxopiperidin-3-yl)-1-oxoisoindolin-4-yl)hex-5-yn-1-yl)-2-methoxy-N-(2-((R)-1-methylpyrrolidin-2-yl)-1H-pyrrolo[3,2-c]pyridin-6-yl)benzamide;
4-(4-((1-(2-(2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)benzamide;
4-(4-((1-(2-((rel-S)-2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)benzamide;
4-(4-((1-(2-((rel-R)-2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)benzamide;
6-(1-((1-(2-(2,6-dioxopiperidin-3-yl)-1-oxoisoindolin-4-yl)piperidin-4-yl)methyl)piperidin-4-yl)-N-(2-((R)-1-methylpyrrolidin-2-yl)-1H-pyrrolo[3,2-c]pyridin-6-yl)nicotinamide;
4-(4-((1-(2-(2,6-dioxopiperidin-3-yl)-1-oxoisoindolin-4-yl)piperidin-4-yl)methyl)piperazin-1-yl)-2-fluoro-6-methoxy-N-(2-((R)-1-methylpyrrolidin-2-yl)-1H-pyrrolo[3,2-c]pyridin-6-yl)benzamide;
4-((1-((1-(2-(2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
4-((1-((1-(2-((rel-S)-2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
4-((1-((1-(2-((rel-R)-2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
4-((1-((1-(2-(2,6-dioxopiperidin-3-yl)-1,3-dioxoisoindolin-5-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
6-((1-((1-(2-(2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)nicotinamide;
6-(4-((1-(2-(2,6-dioxopiperidin-3-yl)-1-oxoisoindolin-4-yl)piperidin-4-yl)methyl)piperazin-1-yl)-2-methoxy-N-(2-((R)-1-methylpyrrolidin-2-yl)-1H-pyrrolo[3,2-c]pyridin-6-yl)nicotinamide;
6-((2S)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)nicotinamide;
4-(3-(1-(2-(2,6-dioxopiperidin-3-yl)-1,3-dioxoisoindolin-4-yl)piperidin-4-yl)propyl)-N-(2-((R)-1-methylpyrrolidin-2-yl)-1H-pyrrolo[3,2-c]pyridin-6-yl)benzamide;
6-(4-(4-(2-(2,6-dioxopiperidin-3-yl)-1-oxoisoindolin-4-yl)benzyl)piperazin-1-yl)-N-(2-((R)-1-methylpyrrolidin-2-yl)-1H-pyrrolo[3,2-c]pyridin-6-yl)nicotinamide;
4-(3-(4-(2-(2,6-dioxopiperidin-3-yl)-1,3-dioxoisoindolin-4-yl)piperazin-1-yl)propyl)-N-(2-((R)-1-methylpyrrolidin-2-yl)-1H-pyrrolo[3,2-c]pyridin-6-yl)benzamide;
4-(3-(4-(2-(2,6-dioxopiperidin-3-yl)-1-oxoisoindolin-4-yl)piperazin-1-yl)propyl)-N-(2-((R)-1-methylpyrrolidin-2-yl)-1H-pyrrolo[3,2-c]pyridin-6-yl)benzamide;
4-((2S)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide;
4-((2R)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide;
4-((R)-4-((1-(2-((rel-R)-2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide;
4-((R)-4-((1-(2-((rel-S)-2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide;
4-(4-((1-(2-(2,6-dioxopiperidin-3-yl)-1,3-dioxoisoindolin-4-yl)piperidin-4-yl)oxy)piperidin-1-yl)-N-(2-((R)-1-methylpyrrolidin-2-yl)-1H-pyrrolo[3,2-c]pyridin-6-yl)benzamide;
4-(4-((1-(2-(2,6-dioxopiperidin-3-yl)-1-oxoisoindolin-4-yl)piperidin-4-yl)oxy)piperidin-1-yl)-N-(2-((R)-1-methylpyrrolidin-2-yl)-1H-pyrrolo[3,2-c]pyridin-6-yl)benzamide;
6-(4-(2-(4-(2-(2,6-dioxopiperidin-3-yl)-1-oxoisoindolin-4-yl)piperazin-1-yl)ethyl)piperidin-1-yl)-N-(2-((R)-1-methylpyrrolidin-2-yl)-1H-pyrrolo[3,2-c]pyridin-6-yl)nicotinamide;
6-(4-((1-(2-(2,6-dioxopiperidin-3-yl)-1-oxoisoindolin-5-yl)piperidin-4-yl)oxy)piperidin-1-yl)-N-(2-((R)-1-methylpyrrolidin-2-yl)-1H-pyrrolo[3,2-c]pyridin-6-yl)nicotinamide;
6-(4-(((1-(2-(2,6-dioxopiperidin-3-yl)-1-oxoisoindolin-4-yl)piperidin-4-yl)oxy)methyl)piperidin-1-yl)-N-(2-((R)-1-methylpyrrolidin-2-yl)-1H-pyrrolo[3,2-c]pyridin-6-yl)nicotinamide;
4-(2-(4-(2-(2,6-dioxopiperidin-3-yl)-1-oxoisoindolin-4-yl)piperidin-1-yl)ethoxy)-N-(2-((R)-1-methylpyrrolidin-2-yl)-1H-pyrrolo[3,2-c]pyridin-6-yl)benzamide;
4-(2-(4-(2-(2,6-dioxopiperidin-3-yl)-1-oxoisoindolin-4-yl)piperazin-1-yl)ethoxy)-N-(2-((R)-1-methylpyrrolidin-2-yl)-1H-pyrrolo[3,2-c]pyridin-6-yl)benzamide;
4-((1-((1-(2-(2,6-dioxopiperidin-3-yl)-1-oxoisoindolin-5-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
4-((1-((1-(2-(2,6-dioxopiperidin-3-yl)-1,3-dioxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
6-(4-((1-(2-(2,6-dioxopiperidin-3-yl)-1-oxoisoindolin-4-yl)piperidin-4-yl)oxy)piperidin-1-yl)-N-(2-((R)-1-methylpyrrolidin-2-yl)-1H-pyrrolo[3,2-c]pyridin-6-yl)nicotinamide;
4-(4-((1-(2-(2,6-dioxopiperidin-3-yl)-1-oxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)benzamide;
6-(4-((1-(2-(2,6-dioxopiperidin-3-yl)-1-oxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)nicotinamide;
6-[4-[[1-[2-(2,6-dioxo-3-piperidyl)-1,3-dioxo-isoindolin-4-yl]-4-piperidyl]oxy]-1-piperidyl]-N-[2-[(2R)-1-methylpyrrolidin-2-yl]-1H-pyrrolo[3,2-c]pyridin-6-yl]pyridine-3-carboxamide;
6-(4-(5-(2-(2,6-dioxopiperidin-3-yl)-1-oxoisoindolin-4-yl)pent-4-yn-1-yl)piperazin-1-yl)-N-(2-((R)-1-methylpyrrolidin-2-yl)-1H-pyrrolo[3,2-c]pyridin-6-yl)nicotinamide;
6-((5-(2-(2,6-dioxopiperidin-3-yl)-1-oxoisoindolin-4-yl)pent-4-yn-1-yl)oxy)-N-(2-((R)-1-methylpyrrolidin-2-yl)-1H-pyrrolo[3,2-c]pyridin-6-yl)nicotinamide;
4-(4-((1-(2-(2,6-dioxopiperidin-3-yl)-1,3-dioxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)benzamide;
6-(1′-(2-(2,6-dioxopiperidin-3-yl)-1,3-dioxoisoindolin-4-yl)-[4,4′-bipiperidin]-1-yl)-N-(2-((R)-1-methylpyrrolidin-2-yl)-1H-pyrrolo[3,2-c]pyridin-6-yl)nicotinamide;
6-(4-(3-(2-(2,6-dioxopiperidin-3-yl)-1-oxoisoindolin-4-yl)prop-2-yn-1-yl)piperidin-1-yl)-N-(2-((R)-1-methylpyrrolidin-2-yl)-1H-pyrrolo[3,2-c]pyridin-6-yl)pyridazine-3-carboxamide;
5-(4-(3-(2-(2,6-dioxopiperidin-3-yl)-1-oxoisoindolin-4-yl)prop-2-yn-1-yl)piperidin-1-yl)-N-(2-((R)-1-methylpyrrolidin-2-yl)-1H-pyrrolo[3,2-c]pyridin-6-yl)pyrazine-2-carboxamide;
4-(4-(3-(2-(2,6-dioxopiperidin-3-yl)-1-oxoisoindolin-4-yl)prop-2-yn-1-yl)piperazin-1-yl)-2-fluoro-N-(2-((R)-1-methylpyrrolidin-2-yl)-1H-pyrrolo[3,2-c]pyridin-6-yl)benzamide;
4-(6-(2-(2,6-dioxopiperidin-3-yl)-1-oxoisoindolin-4-yl)hex-5-yn-1-yl)-3,5-difluoro-N-(2-((R)-1-methylpyrrolidin-2-yl)-1H-pyrrolo[3,2-c]pyridin-6-yl)benzamide;
4-((1-((1-(2-(2,6-dioxopiperidin-3-yl)-1,3-dioxoisoindolin-5-yl)piperidin-4-yl)methyl)azetidin-3-yl)oxy)-N-(2-((R)-1-methylpyrrolidin-2-yl)-1H-pyrrolo[3,2-c]pyridin-6-yl)benzamide;
6-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)nicotinamide;
4-((1-(2-(2,6-dioxopiperidin-3-yl)-1-oxoisoindolin-4-yl)piperidin-4-yl)methoxy)-N-(2-((R)-1-methylpyrrolidin-2-yl)-1H-pyrrolo[3,2-c]pyridin-6-yl)benzamide;
N-(3-chloro-2-((R)-1-methylpyrrolidin-2-yl)-1H-pyrrolo[3,2-c]pyridin-6-yl)-4-(2-(1-(2-(2,6-dioxopiperidin-3-yl)-1-oxoisoindolin-4-yl)piperidin-4-yl)ethoxy)benzamide;
N-(3-chloro-2-((R)-1-methylpyrrolidin-2-yl)-1H-pyrrolo[3,2-c]pyridin-6-yl)-4-(2-(1-(2-((rel-S)-2,6-dioxopiperidin-3-yl)-1-oxoisoindolin-4-yl)piperidin-4-yl)ethoxy)benzamide;
N-(3-chloro-2-((R)-1-methylpyrrolidin-2-yl)-1H-pyrrolo[3,2-c]pyridin-6-yl)-4-(2-(1-(2-((rel-R)-2,6-dioxopiperidin-3-yl)-1-oxoisoindolin-4-yl)piperidin-4-yl)ethoxy)benzamide;
6-((2R)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)nicotinamide;
4-(2-(9-(2-(2,6-dioxopiperidin-3-yl)-1-oxoisoindolin-5-yl)-3,9-diazaspiro[5.5]undecan-3-yl)ethoxy)-N-(2-((R)-1-methylpyrrolidin-2-yl)-1H-pyrrolo[3,2-c]pyridin-6-yl)benzamide;
4-(2-(9-(2-(2,6-dioxopiperidin-3-yl)-1-oxoisoindolin-4-yl)-3,9-diazaspiro[5.5]undecan-3-yl)ethoxy)-N-(2-((R)-1-methylpyrrolidin-2-yl)-1H-pyrrolo[3,2-c]pyridin-6-yl)benzamide;
4-(2-(9-(2-(2,6-dioxopiperidin-3-yl)-1,3-dioxoisoindolin-5-yl)-3,9-diazaspiro[5.5]undecan-3-yl)ethoxy)-N-(2-((R)-1-methylpyrrolidin-2-yl)-1H-pyrrolo[3,2-c]pyridin-6-yl)benzamide;
4-(1-((1-(2-(2,6-dioxopiperidin-3-yl)-1-oxoisoindolin-4-yl)piperidin-4-yl)methyl)piperidin-4-yl)-N-(2-((R)-1-methylpyrrolidin-2-yl)-1H-pyrrolo[3,2-c]pyridin-6-yl)benzamide;
6-(4-((1-(2-(2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)nicotinamide;
4-(4-((1-(1-(2,6-dioxopiperidin-3-yl)-3-methyl-2-oxo-2,3-dihydro-1H-benzo[d]imidazol-5-yl)piperidin-4-yl)methyl)piperazin-1-yl)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide;
4-(2-(1-(2-((rel-S)-2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide;
4-(2-(1-(2-((rel-R)-2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide;
4-(3-(1-(2-(2,6-dioxopiperidin-3-yl)-1-oxoisoindolin-4-yl)piperidin-4-yl)propyl)-2-fluoro-N-(2-((R)-1-methylpyrrolidin-2-yl)-1H-pyrrolo[3,2-c]pyridin-6-yl)benzamide;
4-(3-(1-(2-((rel-S)-2,6-dioxopiperidin-3-yl)-1-oxoisoindolin-4-yl)piperidin-4-yl)propyl)-2-fluoro-N-(2-((R)-1-methylpyrrolidin-2-yl)-1H-pyrrolo[3,2-c]pyridin-6-yl)benzamide;
4-(3-(1-(2-((rel-R)-2,6-dioxopiperidin-3-yl)-1-oxoisoindolin-4-yl)piperidin-4-yl)propyl)-2-fluoro-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1-oxoisoindolin-4-yl)piperidin-4-yl)ethyl)-N-(2-((R)-1-methylpyrrolidin-2-yl)-1H-pyrrolo[3,2-c]pyridin-6-yl)benzamide;
6-(4-(4-(2-(2,6-dioxopiperidin-3-yl)-1,3-dioxoisoindolin-4-yl)phenyl)piperidin-1-yl)-N-(2-((R)-1-methylpyrrolidin-2-yl)-1H-pyrrolo[3,2-c]pyridin-6-yl)nicotinamide;
6-(2-(1-(2-(2,6-dioxopiperidin-3-yl)-1,3-dioxoisoindolin-4-yl)piperidin-4-yl)ethoxy)-N-(2-((R)-1-methylpyrrolidin-2-yl)-1H-pyrrolo[3,2-c]pyridin-6-yl)nicotinamide;
4-(4-((1-(2-(2,6-dioxopiperidin-3-yl)-6-fluoro-1,3-dioxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide;
N—((S)-2,6-dioxopiperidin-3-yl)-5-(4-((4-(4-((2-((R)-1-methylpyrrolidin-2-yl)-1H-pyrrolo[3,2-c]pyridin-6-yl)carbamoyl)phenyl)piperazin-1-yl)methyl)piperidin-1-yl)picolinamide;
N—((S)-2,6-dioxopiperidin-3-yl)-6-(4-((4-(4-((2-((R)-1-methylpyrrolidin-2-yl)-1H-pyrrolo[3,2-c]pyridin-6-yl)carbamoyl)phenyl)piperazin-1-yl)methyl)piperidin-1-yl)picolinamide;
4-(3-((4-(2-(2,6-dioxopiperidin-3-yl)-1,3-dioxoisoindolin-5-yl)piperazin-1-yl)methyl)azetidin-1-yl)-N-(2-((R)-1-methylpyrrolidin-2-yl)-1H-pyrrolo[3,2-c]pyridin-6-yl)benzamide;
4-((1-((1-(2-(2,6-dioxopiperidin-3-yl)-6-fluoro-1,3-dioxoisoindolin-5-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
6-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)pyridazine-3-carboxamide;
5-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)pyrazine-2-carboxamide;
6-((3S)-3-(3-(2-(2,6-dioxopiperidin-3-yl)-1-oxoisoindolin-4-yl)prop-2-yn-1-yl)pyrrolidin-1-yl)-N-(2-((R)-1-methylpyrrolidin-2-yl)-1H-pyrrolo[3,2-c]pyridin-6-yl)nicotinamide;
6-(4-(6-(2-(2,6-dioxopiperidin-3-yl)-1-oxoisoindolin-4-yl)hex-5-yn-1-yl)piperidin-1-yl)-N-(2-((R)-1-methylpyrrolidin-2-yl)-1H-pyrrolo[3,2-c]pyridin-6-yl)nicotinamide;
4-(4-(3-(1-(2-(2,6-dioxopiperidin-3-yl)-1-oxoisoindolin-4-yl)piperidin-4-yl)propanoyl)piperazin-1-yl)-N-(2-((R)-1-methylpyrrolidin-2-yl)-1H-pyrrolo[3,2-c]pyridin-6-yl)benzamide;
N-(3-chloro-2-((R)-1-methylpyrrolidin-2-yl)-1H-pyrrolo[3,2-c]pyridin-6-yl)-4-(2-(1-(2-(2,6-dioxopiperidin-3-yl)-1-oxoisoindolin-4-yl)piperidin-4-yl)ethoxy)-2-fluorobenzamide;
N-(3-chloro-2-((R)-1-methylpyrrolidin-2-yl)-1H-pyrrolo[3,2-c]pyridin-6-yl)-6-(4-((1-(2-(2,6-dioxopiperidin-3-yl)-1-oxoisoindolin-4-yl)piperidin-4-yl)methoxy)piperidin-1-yl)nicotinamide;
3-(4-((1-(2-(2,6-dioxopiperidin-3-yl)-1-oxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide;
6-(4-(2-(1-(2-(2,6-dioxopiperidin-3-yl)-1-oxoisoindolin-4-yl)piperidin-4-yl)ethyl)piperazin-1-yl)-N-(2-((R)-1-methylpyrrolidin-2-yl)-1H-pyrrolo[3,2-c]pyridin-6-yl)nicotinamide;
4-((4-((1-(2-(2,6-dioxopiperidin-3-yl)-1-oxoisoindolin-4-yl)piperidin-4-yl)methyl)piperazin-1-yl)methyl)-N-(2-((R)-1-methylpyrrolidin-2-yl)-1H-pyrrolo[3,2-c]pyridin-6-yl)benzamide;
6-(4-((3-(2-(2,6-dioxopiperidin-3-yl)-1-oxoisoindolin-4-yl)propa-1,2-dien-1-yl)oxy)piperidin-1-yl)-N-(2-((R)-1-methylpyrrolidin-2-yl)-1H-pyrrolo[3,2-c]pyridin-6-yl)nicotinamide;
6-(4-(4-(2-(2,6-dioxopiperidin-3-yl)-1-oxoisoindolin-4-yl)-3-methylphenoxy)piperidin-1-yl)-N-(2-((R)-1-methylpyrrolidin-2-yl)-1H-pyrrolo[3,2-c]pyridin-6-yl)nicotinamide;
5-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)picolinamide;
4-(3-(4-(2-(2,6-dioxopiperidin-3-yl)-1-oxoisoindolin-4-yl)piperidin-1-yl)propyl)-N-(2-((R)-1-methylpyrrolidin-2-yl)-1H-pyrrolo[3,2-c]pyridin-6-yl)benzamide;
4-(3-(4-(2-(2,6-dioxopiperidin-3-yl)-1-oxoisoindolin-4-yl)phenyl)propyl)-N-(2-((R)-1-methylpyrrolidin-2-yl)-1H-pyrrolo[3,2-c]pyridin-6-yl)benzamide;
4-((7-(2-(2,6-dioxopiperidin-3-yl)-1-oxoisoindolin-4-yl)-7-azaspiro[3.5]nonan-2-yl)oxy)-2-fluoro-N-(2-((R)-1-methylpyrrolidin-2-yl)-1H-pyrrolo[3,2-c]pyridin-6-yl)benzamide;
4-((7-(2-(2,6-dioxopiperidin-3-yl)-1-oxoisoindolin-4-yl)-7-azaspiro[3.5]nonan-2-yl)methoxy)-2-fluoro-N-(2-((R)-1-methylpyrrolidin-2-yl)-1H-pyrrolo[3,2-c]pyridin-6-yl)benzamide;
4-(9-(2-(2,6-dioxopiperidin-3-yl)-1-oxoisoindolin-4-yl)-3,9-diazaspiro[5.5]undecan-3-yl)-2-fluoro-N-(2-((R)-1-methylpyrrolidin-2-yl)-1H-pyrrolo[3,2-c]pyridin-6-yl)benzamide;
4-(4-(4-(2-(2,6-dioxopiperidin-3-yl)-1-oxoisoindolin-4-yl)-3-methylphenoxy)piperidin-1-yl)-2-fluoro-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1-oxoisoindolin-4-yl)piperidin-4-yl)ethoxy)-2-methyl-N-(2-((R)-1-methylpyrrolidin-2-yl)-1H-pyrrolo[3,2-c]pyridin-6-yl)benzamide;
2-chloro-4-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1-oxoisoindolin-4-yl)piperidin-4-yl)ethoxy)-3-fluoro-N-(2-((R)-1-methylpyrrolidin-2-yl)-1H-pyrrolo[3,2-c]pyridin-6-yl)benzamide;
4-(4-(2-(2,6-dioxopiperidin-3-yl)-1-oxoisoindolin-4-yl)phenethoxy)-2-fluoro-N-(2-((R)-1-methylpyrrolidin-2-yl)-1H-pyrrolo[3,2-c]pyridin-6-yl)benzamide;
4-(4-(2-(2,6-dioxopiperidin-3-yl)-1-oxoisoindolin-4-yl)-3-methylphenethoxy)-2-fluoro-N-(2-((R)-1-methylpyrrolidin-2-yl)-1H-pyrrolo[3,2-c]pyridin-6-yl)benzamide;
4-(3-(1-(2-(2,6-dioxopiperidin-3-yl)-1-oxoisoindolin-4-yl)piperidin-4-yl)propoxy)-2-fluoro-N-(2-((R)-1-methylpyrrolidin-2-yl)-1H-pyrrolo[3,2-c]pyridin-6-yl)benzamide;
4-(1-(3-(1-(2-(2,6-dioxopiperidin-3-yl)-1,3-dioxoisoindolin-4-yl)piperidin-4-yl)propyl)-1H-pyrazol-4-yl)-2-fluoro-N-(2-((R)-1-methylpyrrolidin-2-yl)-1H-pyrrolo[3,2-c]pyridin-6-yl)benzamide;
5-(1-(3-(1-(2-(2,6-dioxopiperidin-3-yl)-1,3-dioxoisoindolin-4-yl)piperidin-4-yl)propyl)-1H-pyrazol-4-yl)-N-(2-((R)-1-methylpyrrolidin-2-yl)-1H-pyrrolo[3,2-c]pyridin-6-yl)picolinamide;
4-(1-(3-(1-(2-(2,6-dioxopiperidin-3-yl)-1-oxoisoindolin-4-yl)piperidin-4-yl)propyl)-1H-pyrazol-4-yl)-2-fluoro-N-(2-((R)-1-methylpyrrolidin-2-yl)-1H-pyrrolo[3,2-c]pyridin-6-yl)benzamide;
4-((5-(2-(2,6-dioxopiperidin-3-yl)-1-oxoisoindolin-4-yl)pent-4-yn-1-yl)oxy)-2-fluoro-N-(2-((R)-1-methylpyrrolidin-2-yl)-1H-pyrrolo[3,2-c]pyridin-6-yl)benzamide;
4-((1-(2-(2,6-dioxopiperidin-3-yl)-1-oxoisoindolin-4-yl)piperidin-4-yl)methoxy)-2-fluoro-N-(2-((R)-1-methylpyrrolidin-2-yl)-1H-pyrrolo[3,2-c]pyridin-6-yl)benzamide;
N—((S)-2,6-dioxopiperidin-3-yl)-2-fluoro-4-(4-(2-(3-fluoro-4-((2-((R)-1-methylpyrrolidin-2-yl)-1H-pyrrolo[3,2-c]pyridin-6-yl)carbamoyl)phenoxy)ethyl)piperidin-1-yl)benzamide;
5-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)picolinamide;
4-(2-(1-(2-(2,6-dioxopiperidin-3-yl)-1,3-dioxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide;
4-(4-(4-(2-(2,6-dioxopiperidin-3-yl)-1-oxoisoindolin-4-yl)phenyl)piperidin-1-yl)-2-fluoro-N-(2-((R)-1-methylpyrrolidin-2-yl)-1H-pyrrolo[3,2-c]pyridin-6-yl)benzamide;
N—((S)-2,6-dioxopiperidin-3-yl)-2-fluoro-3-(5-(4-((2-((R)-1-methylpyrrolidin-2-yl)-1H-pyrrolo[3,2-c]pyridin-6-yl)carbamoyl)phenyl)pent-1-yn-1-yl)benzamide;
3-(4-((1-(2-(2,6-dioxopiperidin-3-yl)-1,3-dioxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide;
4-(4-((1-(2-(2,6-dioxopiperidin-3-yl)-1,3-dioxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide;
4-(4-((1-(2-(2,6-dioxopiperidin-3-yl)-1-oxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide;
4-(6-(2-(2,6-dioxopiperidin-3-yl)-1-oxoisoindolin-5-yl)hex-5-yn-1-yl)-2-fluoro-N-(2-((R)-1-methylpyrrolidin-2-yl)-1H-pyrrolo[3,2-c]pyridin-6-yl)benzamide;
6-(4-(3-(2-(2,6-dioxopiperidin-3-yl)-1-oxoisoindolin-5-yl)prop-2-yn-1-yl)piperidin-1-yl)-N-(2-((R)-1-methylpyrrolidin-2-yl)-1H-pyrrolo[3,2-c]pyridin-6-yl)nicotinamide;
4-(5-(2-(2,6-dioxopiperidin-3-yl)-3-oxoisoindolin-5-yl)pent-4-yn-1-yl)-N-(2-((R)-1-methylpyrrolidin-2-yl)-1H-pyrrolo[3,2-c]pyridin-6-yl)benzamide;
6-((3R)-3-(3-(2-(2,6-dioxopiperidin-3-yl)-1-oxoisoindolin-4-yl)prop-2-yn-1-yl)pyrrolidin-1-yl)-N-(2-((R)-1-methylpyrrolidin-2-yl)-1H-pyrrolo[3,2-c]pyridin-6-yl)nicotinamide;
4-((1-(2-(1-(2-(2,6-dioxopiperidin-3-yl)-1,3-dioxoisoindolin-5-yl)piperidin-4-yl)ethyl)azetidin-3-yl)oxy)-N-(2-((R)-1-methylpyrrolidin-2-yl)-1H-pyrrolo[3,2-c]pyridin-6-yl)benzamide;
6-(4-((1-(2-(2,6-dioxopiperidin-3-yl)-1-oxoisoindolin-5-yl)piperidin-4-yl)methoxy)piperidin-1-yl)-N-(2-((R)-1-methylpyrrolidin-2-yl)-1H-pyrrolo[3,2-c]pyridin-6-yl)nicotinamide;
N-(3-chloro-2-((R)-1-methylpyrrolidin-2-yl)-1H-pyrrolo[3,2-c]pyridin-6-yl)-4-(2-(1-(2-(2,6-dioxopiperidin-3-yl)-1-oxoisoindolin-4-yl)piperidin-4-yl)ethoxy)-3-fluorobenzamide;
4-(2-(1-(2-(2,6-dioxopiperidin-3-yl)-1-oxoisoindolin-4-yl)piperidin-4-yl)ethoxy)-2,5-difluoro-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1-oxoisoindolin-4-yl)piperidin-4-yl)ethoxy)-2,3-difluoro-N-(2-((R)-1-methylpyrrolidin-2-yl)-1H-pyrrolo[3,2-c]pyridin-6-yl)benzamide;
4-(3-(1-(2-((S)-2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide; and
N-[3-chloro-2-[(R)-1-methylpyrrolidin-2-yl]-1H-pyrrolo[3,2-c]pyridin-6-yl]-4-[3-[1-[2-[(rel-S)-2,6-dioxo-3-piperidyl]-1-oxo-isoindolin-4-yl]-4-piperidyl]propyl]benzamide.

145. A compound according to any one of aspects 101 to 128, wherein M is of formula (IV):

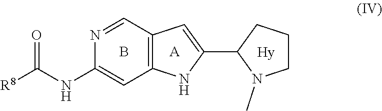

wherein R⁸ is (i) a bond to LINK or (ii) a group selected from a 6-membered aryl ring and a 5-to 6-membered heteroaryl ring, the group R⁸ being unsubstituted or substituted by one, two or three substituents independently selected from halo, C₁₋₄ alkyl and R¹⁰;
R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl, more preferably pyrazolyl and cyclopropyl;
wherein either R⁸ is (i) a bond to LINK, or R⁸ is a group (ii) and M is bonded to LINK via a C or N atom within group R⁸ such that a hydrogen atom on the C or N atom within group R⁸ is replaced with a bond to LINK.

146. A compound according to any one of aspects 101 to 145, wherein R⁸ is phenyl, pyrimidinyl or pyridinyl.

147. A compound according to any one of the preceding aspects, wherein the group R⁸ is unsubstituted or substituted by one fluoro or chloro group and/or by one group R¹⁰.

148. A compound according to any one of the preceding aspects, wherein the group R⁸ is phenyl, pyrimidinyl or pyridinyl which is unsubstituted or substituted by one fluoro or chloro group and/or by one group R¹⁰, wherein R¹⁰ is pyrazolyl, or wherein phenyl, pyrimidinyl or pyridinyl which is unsubstituted or substituted by one fluoro or chloro group.

149. A compound according to any one of aspects 145 to 148, wherein R⁸ is selected from one of the following structures:

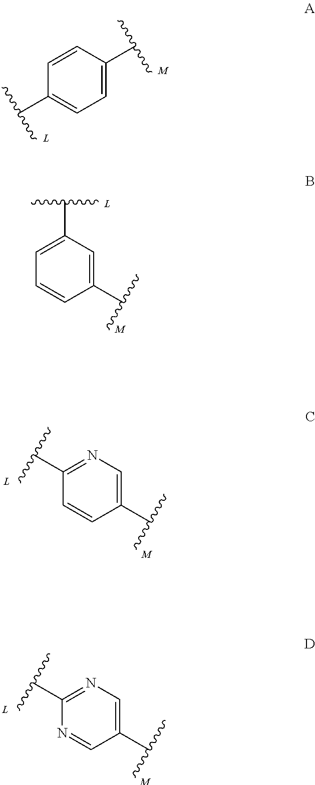

wherein:

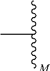

represents the point of attachment of R⁸ to the rest of moiety M;

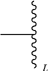

represents the point of attachment of R⁸ to LINK or R¹⁰.

150. A compound according to any one of aspects 145 to 148, wherein R⁸ is selected from one of the following structures:

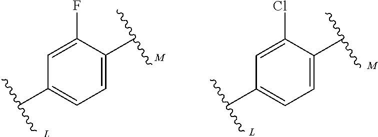

wherein:

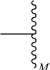

represents the point of attachment of R⁸ to the rest of moiety M;

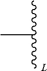

represents the point of attachment of R⁸ to LINK or R¹⁰.

151. A compound according to any one of aspects 101 to 135 and 139 to 143, wherein at least one of R⁴, R⁵ and R⁶ is not H.

152. A compound according to any one of aspects 101 to 139, wherein R⁸ is a bond to link.

153. A compound according to any one of aspects 101 to 139, wherein R⁸ is a group (ii) and M is bonded to LINK via a C or N atom within group R⁸.

154. A compound according to any one of the preceding aspects, wherein the bond from ring Hy to ring A is in the “up” position and the bond from ring Hy to R² is in the “down” position relative to ring Hy, as depicted below:

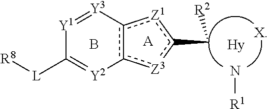

155. A compound according to any one of aspects 101 to 154, wherein the E3 ubiquitin ligase binding moiety (U) has the following structure:

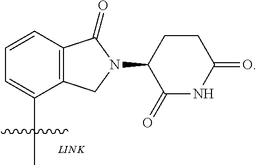 [SMILES: *c1cccc2c1CN([C@H]1CCC(=O)NC1=O)C2=O]

156. A pharmaceutical composition which comprises a compound as defined in any one of aspects 101 to 155 and a pharmaceutically acceptable carrier or diluent.

157. A compound as defined in any one of aspects 101 to 155 for use in the treatment of the human or animal body by therapy.

158. A compound as defined in any one of aspects 101 to 155 for use in the treatment of cancer.

159. The compound for use according to aspect 158, wherein the cancer is a transcriptionally addicted cancer, such as breast cancer, acute leukemia, chronic leukemia, prostate cancer, bladder cancer, cholangiocarcinoma, colon adenocarcinoma, esophageal carcinoma, head and neck squamous cell carcinoma, kidney chromophobe, kidney renal clear cell carcinoma, kidney renal papillary cell carcinoma, liver hepatocellular carcinoma, lung adenocarcinoma, pheochromocytoma and paraganglioma, rectum adenocarcinoma, stomach adenocarcinoma, uterine corpus endometrial carcinoma, cervical squamous cell carcinoma and endocervical adenocarcinoma, lung squamous cell carcinoma, neuroblastoma, midline glioma or sarcoma, preferably neuroblastoma or midline glioma.

160. The compound for use according to aspect 158, wherein the cancer is acute myeloid leukaemia (AML), myelodysplastic syndrome (MDS), myeloproliferative neoplasm (MPN) or acute lymphoblastic leukaemia (ALL).

161. The compound for use according to aspect 160, wherein the cancer is mixed-lineage leukaemia (MLL) rearranged (also known as KMT2Ar) acute leukemia (AML and ALL).

162. The compound for use according to aspect 160, wherein the cancer is NPM1 mutant acute leukemia.

163. The compound for use according to aspect 160, wherein the cancer is RUNX1-fusion acute leukemia.

164. The compound for use according to aspect 160, wherein the cancer is E2A-fusion acute leukemia.

165. The compound for use according to aspect 160, wherein the cancer is PML-fusion acute leukemia.

166. The compound for use according to aspect 160, wherein the cancer is NUP98-fusion acute leukemia.

1. A compound which is a Proteolysis Targeting Chimera (PROTAC) or a pharmaceutically acceptable salt thereof, wherein the PROTAC has the structure: 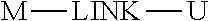
wherein U is an E3 ubiquitin ligase binding moiety, LINK is a moiety that covalently links M and U, and M is an MLLT1 and/or MLLT3 binder of formula (I): 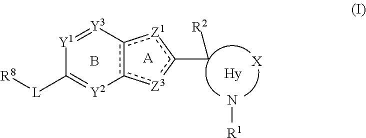
wherein:
one of Z¹ and Z³ is —N(H)— and the other is N or —C(R⁴)—, Y¹ is N, Y² is N or —C(R⁶)—, and Y³ is N or —C(R⁵)—;
Hy is a 4- to 7-membered heterocyclic ring containing X and at least one N atom, wherein: ring Hy is linked to ring A via a C atom within ring Hy, said C atom also being linked to R²; a N atom within ring Hy is substituted by R¹; X is a bond, —N(R¹¹)—, O, S, —S(O)₂—, —S(O)(NR¹¹)—, or —C(R¹¹)₂—; and the rest of ring Hy is unsubstituted or substituted by one or two R³;
L is —C(O)N(H)—, wherein the C atom of L is bonded to R⁸, and the N atom of L is bonded to ring B;
R¹ is H, C₁₋₄ cycloalkyl, or C₁₋₄ alkyl which is itself unsubstituted or substituted with one C₁₋₄ alkoxy or one, two or three halo;
R² is H or methyl;
each R³ is independently selected from C₁₋₄ alkyl, C₁₋₄ alkoxy, phenyl, a 5- to 6-membered heteroaryl ring and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring or a C₃₋₆ heterocycloalkyl ring;
R⁴ and R⁶ are independently selected from H, halo, CN, C₁₋₄ alkoxy, and C₁₋₄ alkyl which is itself unsubstituted or substituted by one, two or three halo;
R⁵ is selected from H, halo, C₁₋₄ alkoxy, C₃₋₅ cycloalkyl and C₁₋₄ alkyl which is itself unsubstituted or substituted by one, two or three halo;
R⁸ is (i) a bond to LINK or (ii) a group selected from a 6-membered aryl ring, a 5- to 6-membered heteroaryl ring, a 5- to 6-membered heterocyclyl ring, and a 5- to 6-membered cycloalkyl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo; and
each R¹¹ is independently selected from H, C₁₋₄ alkyl, and C₁₋₄ cycloalkyl;
wherein either R⁸ is (i) a bond to LINK, or R⁸ is a group (ii) and M is bonded to LINK via a C or N atom within group R⁸ such that a hydrogen atom on the C or N atom within group R⁸ is replaced with a bond to LINK.

2. A compound according to claim 1, wherein LINK is (a) a single bond, or (b) a chemical linker group represented by formula (L): 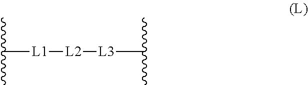
wherein:
LINK is attached to M via L1;
LINK is attached to U via L3;
L1 and L3 are each independently selected from a single bond, —N(R′)—, —C(O)N(R′)—, —O—, —N(R′)C(O)—, —C(O)—, —S(O₂)N(R′)—, —N(R′)S(O₂)—, —(C₁₋₆ alkylene)-, ethynylene, —(C₂₋₆ alkenylene)-, —C═C═C—, phenylene, a divalent 3- to 6-membered cycloalkyl ring and a divalent 4- to 7-membered heterocyclyl ring, the phenylene, cycloalkyl and heterocyclyl rings being unsubstituted or substituted by one or two C₁₋₄ alkyl;
L2 is represented by the formula -(L4)ₘ-;
each L4 is independently a unit of formula 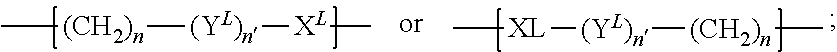
each X^{L} is independently selected from a single bond, —N(R′)—, —O—, —C(O)—, —S—, —SO—, —SO₂—, —C(R″)═C(R″)— and —C≡C—;
each Y^{L} is independently selected from a divalent 3- to 6-membered cycloalkyl ring, a divalent 4- to 7-membered heterocyclyl ring, or one of the following structures: 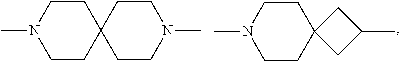
the cycloalkyl and heterocyclyl rings being unsubstituted or substituted by one or two C₁₋₄ alkyl;
n is selected from 0 to 4;
n′ is selected from 0 or 1;
n+n′≥1;
m is selected from 1 to 30;
each R′ is independently selected from H and C₁₋₄ alkyl; and
each R″ is independently selected from H and halo.

3. A compound according to claim 1 or 2, wherein U is:
(a) a CRBN E3 ubiquitin ligase binding moiety of formula (U1): 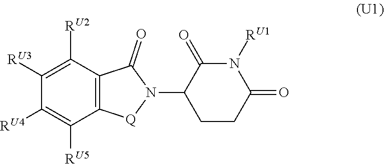
wherein:
R^{U1} is H;
Q is selected from —CH(R^{U6})—, —N(R^{U6})—, —O—, —C(O)—, —NH—CH(R^{U6})—, —N═C(R^{U6})—, or —N═N—;
R^{U6} is H or C₁₋₄ alkyl;
R^{U2} and R^{U5} are each independently selected from H, halogen, C₁₋₄ alkyl, NH₂, NO₂, OH, COOH, CN, CF₃, and a single bond to LINK;
R^{U3} and R^{U4} are each independently selected from H, halogen, C₁₋₄ alkyl, NH₂, NO₂, OH, COOH, CN, CF₃, and a single bond to LINK;
wherein one and only one of R^{U2}, R^{U3}, R^{U4} and R^{U5} is a single bond to LINK;
(b) a CRBN E3 ubiquitin ligase binding moiety of formula (U4): 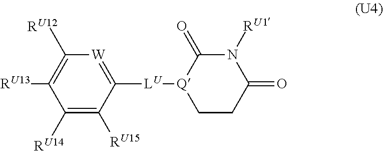
wherein:
R^{U1′} is H;
W is N or CR^{U16};
Q′ is N and L^{U} is a single bond, or Q′ is CH and L^{U} is selected from a single bond or —C(O)N(H)—, wherein either (i) the C atom of L^{U} is bonded to phenyl, and the N atom of L^{U} is bonded to Q′; or (ii) the C atom of L^{U} is bonded to Q′, and the N atom of L^{U} is bonded to phenyl;
R^{U12}, R^{U13} and R^{U14} are each independently selected from H, halogen, C₁₋₄ alkyl, NH₂, NO₂, OH, COOH, CN, CF₃, and a single bond to LINK;
wherein one and only one of R^{U12}, R^{U13} and R^{U14} is a single bond to LINK;
R^{U15} and R^{U16} are each independently selected from H, halogen, C₁₋₄ alkyl, NH₂, NO₂, OH, COOH, CN and CF₃;
(c) a CRBN E3 ubiquitin ligase binding moiety of formula (U5): 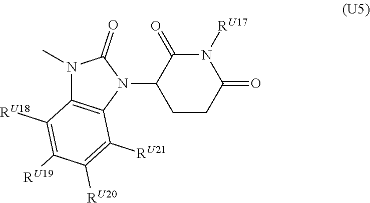
wherein:
R^{U17} is H;
R^{U18}, R^{U19}, R^{U20} and R^{U21} are each independently selected from H, halogen, C₁₋₄ alkyl, NH₂, NO₂, OH, COOH, CN, CF₃, and a single bond to LINK;
wherein one and only one of R^{U18}, R^{U19}, R^{U20} and R^{U21} is a single bond to LINK;
or (d) a VHL E3 ubiquitin ligase binding moiety of formula (U2): 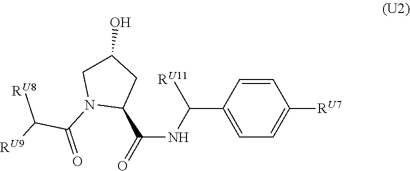
wherein:
R^{U7} is a group selected from phenyl, a 5- to 6-membered heteroaryl ring, a 5- to 6-membered heterocyclyl ring, and a 5- to 6-membered cycloalkyl ring, the group R^{U7} being unsubstituted or substituted by C₁₋₄ alkyl;
R^{U11} is H or C₁₋₄alkyl;
R^{U8} is selected from C₁₋₄ alkyl, phenyl and a 3- to 8-membered cycloalkyl ring; and
R^{U9} is a single bond to LINK.

4. A compound according to any one of the preceding claims, wherein the N atom of ring Hy which is substituted by R¹ is adjacent to the C atom of ring Hy that is bonded to ring A.

5. A compound according to any one of the preceding claims, wherein Z¹ is —C(R⁴)—, Z³ is —N(H)—, Y¹ is N, Y² is —C(R⁶)— and Y³ is —C(R⁵)—.

6. A compound according to any one of the preceding claims, wherein M is of formula (II): 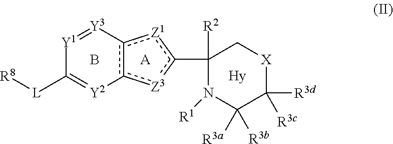
wherein:
Z¹, Z³, Y¹, Y², Y³, R¹, R², R⁸, X and L are as defined in any one of the preceding claims;
R^{3a} and R^{3b} are independently selected from H, C₁₋₄ alkyl, phenyl, a 5- to 6-membered heteroaryl ring and C₁₋₄ alkoxy, or R^{3a} and R^{3b} form, together with the C atom to which they are attached, a C₃₋₆ cycloalkyl ring or a C₃₋₆ heterocycloalkyl ring,
R^{3c} and R^{3d} are independently selected from H, C₁₋₄ alkyl, halo, phenyl, a 5- to 6-membered heteroaryl ring and C₁₋₄ alkoxy, or R^{3c} and R^{3d} form, together with the C atom to which they are attached, a C₃₋₆ cycloalkyl ring or a C₃₋₆ heterocycloalkyl ring, or
R^{3a} and R^{3c} are H, and R^{3b} and R^{3d} form, together with the C atoms to which they are attached, a C₅₋₆ cycloalkyl ring, with the proviso that when X is —N(R¹¹)—, O, S, —S(O)₂— or —S(O)(NR¹¹)-then neither R^{3c} nor R^{3d} are halo;
wherein at least two of R^{3a}, R^{3b}, R^{3c} and R^{3d} are H; and
wherein either (i) R⁸ is a bond to LINK, or (ii) R⁸ is a group and M is bonded to LINK via a C or N atom within group R⁸ such that a hydrogen atom on the C or N atom within group R⁸ is replaced with a bond to LINK.

7. A compound according to any one of the preceding claims, wherein M is of formula (III): 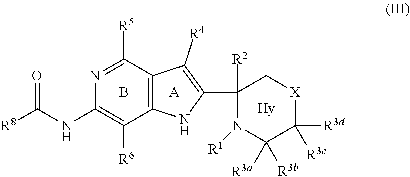
wherein:
R¹ is H or C₁₋₄ alkyl which is itself unsubstituted or substituted with one C₁₋₄ alkoxy or one, two or three halo, preferably with one C₁₋₄ alkoxy;
R² is H or methyl;
R^{3a} and R^{3b} are independently selected from H, C₁₋₄ alkyl, and C₁₋₄ alkoxy, or R^{3a} and R^{3b} form, together with the C atom to which they are attached, a C₃₋₆ cycloalkyl ring,
R^{3c} and R^{3d} are independently selected from H, C₁₋₄ alkyl, halo and C₁₋₄ alkoxy, or R^{3c} and R^{3d} form, together with the C atom to which they are attached, a C₃₋₆ cycloalkyl ring, or
R^{3a} and R^{3c} are H, and R^{3b} and R^{3d} form, together with the C atoms to which they are attached, a C₅₋₆ cycloalkyl ring,
with the proviso that when X is —N(Me)- or O, then neither R^{3c} nor R^{3d} are halo;
wherein at least two of R^{3a}, R^{3b}, R^{3c} and R^{3d} are H;
X is a bond, —N(Me)-, O or —CH₂—;
R⁴ is selected from H, CN, halo, C₁₋₄ alkoxy, and C₁₋₄ alkyl which is itself unsubstituted or substituted by one, two or three halo;
R⁵ and R⁶ are independently selected from H, halo, C₁₋₄ alkoxy, and C₁₋₄ alkyl which is itself unsubstituted or substituted by one, two or three halo;
R⁸ is (i) a bond to LINK or (ii) a group selected from a 6-membered aryl ring, a 5- to 6-membered heteroaryl ring, and a 5- to 6-membered heterocyclyl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo; and
wherein either R⁸ is (i) a bond to LINK, or R⁸ is a group (ii) and M is bonded to LINK via a C or N atom within group R⁸ such that a hydrogen atom on the C or N atom within group R⁸ is replaced with a bond to LINK.

8. A compound according to any one of the preceding claims, wherein Hy is an unsubstituted pyrrolidine ring, R¹ is methyl, and R² is H.

9. A compound according to claim 1, wherein the PROTAC structure is selected from:
4-(4-(2-(2,6-dioxopiperidin-3-yl)-1-oxoisoindolin-4-yl)but-3-yn-1-yl)-N-(2-((R)-1-methylpyrrolidin-2-yl)-1H-pyrrolo[3,2-c]pyridin-6-yl)piperidine-1-carboxamide;
6-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)nicotinamide;
4-((1-(2-(2,6-dioxopiperidin-3-yl)-1,3-dioxoisoindolin-4-yl)piperidin-4-yl)methyl)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1,3-dioxoisoindolin-4-yl)piperidin-4-yl)ethyl)-N-(2-((R)-1-methylpyrrolidin-2-yl)-1H-pyrrolo[3,2-c]pyridin-6-yl)benzamide;
6-(4-((1-(2-(2,6-dioxopiperidin-3-yl)-1,3-dioxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)nicotinamide;
6-(4-(3-(2-(2,6-dioxopiperidin-3-yl)-1-oxoisoindolin-4-yl)prop-2-yn-1-yl)piperazin-1-yl)-N-(2-((R)-1-methylpyrrolidin-2-yl)-1H-pyrrolo[3,2-c]pyridin-6-yl)nicotinamide;
N—((S)-2,6-dioxopiperidin-3-yl)-6-(6-(4-((2-((R)-1-methylpyrrolidin-2-yl)-1H-pyrrolo[3,2-c]pyridin-6-yl)carbamoyl)phenyl)hex-1-yn-1-yl)picolinamide;
4-(6-(2-(2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide;
(R)-4-(6-(6-(2,4-dioxotetrahydropyrimidin-1(2H)-yl)pyridin-2-yl)hex-5-yn-1-yl)-N-(2-(1-methylpyrrolidin-2-yl)-1H-pyrrolo[3,2-c]pyridin-6-yl)benzamide;
6-(6-(2-(2,6-dioxopiperidin-3-yl)-1-oxoisoindolin-4-yl) hex-5-yn-1-yl)-N-(2-((R)-1-methylpyrrolidin-2-yl)-1H-pyrrolo[3,2-c]pyridin-6-yl) nicotinamide;
N—((S)-2,6-dioxopiperidin-3-yl)-5-(5-(4-((2-((R)-1-methylpyrrolidin-2-yl)-1H-pyrrolo[3,2-c]pyridin-6-yl)carbamoyl)phenyl)pent-1-yn-1-yl)picolinamide;
6-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)nicotinamide;
N-(3-chloro-2-((R)-1-methylpyrrolidin-2-yl)-1H-pyrrolo[3,2-c]pyridin-6-yl)-9-(2-(2,6-dioxopiperidin-3-yl)-1-oxoisoindolin-4-yl) non-8-ynamide;
3-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)benzamide;
4-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)benzamide;
6-(4-(3-(2-(2,6-dioxopiperidin-3-yl)-1-oxoisoindolin-4-yl)prop-2-yn-1-yl)piperidin-1-yl)-N-(2-((R)-1-methylpyrrolidin-2-yl)-1H-pyrrolo[3,2-c]pyridin-6-yl)nicotinamide;
6-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)nicotinamide;
6-(4-((1-(2-(2,6-dioxopiperidin-3-yl)-1,3-dioxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)nicotinamide;
4-((4-(2-(2,6-dioxopiperidin-3-yl)-1-oxoisoindolin-4-yl)but-3-yn-1-yl)oxy)-N-(2-((R)-1-methylpyrrolidin-2-yl)-1H-pyrrolo[3,2-c]pyridin-6-yl)benzamide;
4-(1-(5-(2-(2,6-dioxopiperidin-3-yl)-1-oxoisoindolin-4-yl)pent-4-yn-1-yl)-1H-pyrazol-3-yl)-N-(2-((R)-1-methylpyrrolidin-2-yl)-1H-pyrrolo[3,2-c]pyridin-6-yl)benzamide;
4-(1-(5-(2-(2,6-dioxopiperidin-3-yl)-1-oxoisoindolin-4-yl)pent-4-yn-1-yl)-1H-pyrazol-4-yl)-N-(2-((R)-1-methylpyrrolidin-2-yl)-1H-pyrrolo[3,2-c]pyridin-6-yl)benzamide;
4-(1-(5-(2-(2,6-dioxopiperidin-3-yl)-1-oxoisoindolin-5-yl)pent-4-yn-1-yl)-1H-pyrazol-4-yl)-N-(2-((R)-1-methylpyrrolidin-2-yl)-1H-pyrrolo[3,2-c]pyridin-6-yl)benzamide;
3-chloro-4-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)benzamide;
4-(((2S)-4-(2-(2,6-dioxopiperidin-3-yl)-1-oxoisoindolin-4-yl)but-3-yn-2-yl)oxy)-N-(2-((R)-1-methylpyrrolidin-2-yl)-1H-pyrrolo[3,2-c]pyridin-6-yl)benzamide;
4-(((2R)-4-(2-(2,6-dioxopiperidin-3-yl)-1-oxoisoindolin-4-yl)but-3-yn-2-yl)oxy)-N-(2-((R)-1-methylpyrrolidin-2-yl)-1H-pyrrolo[3,2-c]pyridin-6-yl)benzamide;
3-((9-(2-(2,6-dioxopiperidin-3-yl)-1,3-dioxoisoindolin-4-yl)non-8-yn-1-yl)oxy)-N-(2-((R)-1-methylpyrrolidin-2-yl)-1H-pyrrolo[3,2-c]pyridin-6-yl)benzamide;
7-{[2-(2,6-dioxopiperidin-3-yl)-1,3-dioxoisoindol-5-yl]amino}-N-{2-[(2R)-1-methylpyrrolidin-2-yl]-1H-pyrrolo[3,2-c]pyridin-6-yl}heptanamide;
7-{[2-(2,6-dioxopiperidin-3-yl)-1,3-dioxoisoindol-5-yl]amino}-N-{2-[(2S)-1-methylpyrrolidin-2-yl]-1H-pyrrolo[3,2-c]pyridin-6-yl}heptanamide;
7-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}heptanamide;
9-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}nonanamide;
12-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}dodecanamide;
12-{[2-(2,6-dioxopiperidin-3-yl)-1,3-dioxoisoindol-4-yl]amino}-N-{2-[(2S)-1-methylpyrrolidin-2-yl]-1H-pyrrolo[3,2-c]pyridin-6-yl}dodecanamide;
N-{3-chloro-2-[(2R)-1-methylpyrrolidin-2-yl]-1H-pyrrolo[3,2-c]pyridin-6-yl}-7-{[2-(2,6-dioxopiperidin-3-yl)-1,3-dioxoisoindol-4-yl]amino}heptanamide;
4-[1-(3-{1-[2-(2,6-dioxopiperidin-3-yl)-1,3-dioxoisoindol-4-yl]piperidin-4-yl}propyl)pyrazol-4-yl]-N-{2-[(2R)-1-methylpyrrolidin-2-yl]-1H-pyrrolo[3,2-c]pyridin-6-yl}benzamide;
4-[1-(3-{1-[2-(2,6-dioxopiperidin-3-yl)-1-oxo-3H-isoindol-4-yl]piperidin-4-yl}propyl)pyrazol-4-yl]-N-{2-[(2R)-1-methylpyrrolidin-2-yl]-1H-pyrrolo[3,2-c]pyridin-6-yl}benzamide;
4-(3-{[2-(2,6-dioxopiperidin-3-yl)-1,3-dioxoisoindol-4-yl]amino}propyl)-N-{2-[(2R)-1-methylpyrrolidin-2-yl]-1H-pyrrolo[3,2-c]pyridin-6-yl}benzamide;
4-(3-{[2-(2,6-dioxopiperidin-3-yl)-1,3-dioxoisoindol-4-yl]amino}propyl)-N-{2-[(2S)-1-methylpyrrolidin-2-yl]-1H-pyrrolo[3,2-c]pyridin-6-yl}benzamide;
1-(3-{1-[2-(2,6-dioxopiperidin-3-yl)-1,3-dioxoisoindol-4-yl]piperidin-4-yl}propyl)-N-{2-[(2R)-1-methylpyrrolidin-2-yl]-1H-pyrrolo[3,2-c]pyridin-6-yl}pyrazole-4-carboxamide;
2-(((1r,4R)-4-((2-(2,6-dioxopiperidin-3-yl)-1,3-dioxoisoindolin-4-yl)amino)cyclohexyl)oxy)-N-(2-((R)-1-methylpyrrolidin-2-yl)-1H-pyrrolo[3,2-c]pyridin-6-yl)acetamide;
2-(4-((2-(2,6-dioxopiperidin-3-yl)-1,3-dioxoisoindolin-4-yl)amino)butoxy)-N-(2-((R)-1-methylpyrrolidin-2-yl)-1H-pyrrolo[3,2-c]pyridin-6-yl)acetamide;
4-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)butanamide;
5-(1-(2-(2,6-Dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)pentanamide;
6-(1-(2-(2,6-dioxopiperidin-3-yl)-1,3-dioxoisoindolin-4-yl)piperidin-4-yl)-N-(2-((R)-1-methylpyrrolidin-2-yl)-1H-pyrrolo[3,2-c]pyridin-6-yl)hexanamide;
4-(1-(2-(2,6-dioxopiperidin-3-yl)-1,3-dioxoisoindolin-4-yl)piperidin-3-yl)-N-(2-((R)-1-methylpyrrolidin-2-yl)-1H-pyrrolo[3,2-c]pyridin-6-yl)butanamide;
3-(3-((2-(2,6-dioxopiperidin-3-yl)-1,3-dioxoisoindolin-4-yl)amino)propyl)-N-(2-((R)-1-methylpyrrolidin-2-yl)-1H-pyrrolo[3,2-c]pyridin-6-yl)benzamide;
3-(((1-(2-(2,6-dioxopiperidin-3-yl)-1,3-dioxoisoindolin-4-yl)piperidin-4-yl)methyl)amino)-N-(2-((R)-1-methylpyrrolidin-2-yl)-1H-pyrrolo[3,2-c]pyridin-6-yl)benzamide;
3-(1-(3-(1-(2-(2,6-dioxopiperidin-3-yl)-1,3-dioxoisoindolin-4-yl)piperidin-4-yl)propyl)-1H-pyrazol-4-yl)-N-(2-((R)-1-methylpyrrolidin-2-yl)-1H-pyrrolo[3,2-c]pyridin-6-yl)benzamide;
4-(1-(2-(2,6-dioxopiperidin-3-yl)-1,3-dioxoisoindolin-4-yl)azetidin-3-yl)-N-(2-((R)-1-methylpyrrolidin-2-yl)-1H-pyrrolo[3,2-c]pyridin-6-yl)butanamide;
5-(4-(2-(2,6-dioxopiperidin-3-yl)-1-oxoisoindolin-4-yl)but-3-yn-1-yl)-N-(2-((R)-1-methylpyrrolidin-2-yl)-1H-pyrrolo[3,2-c]pyridin-6-yl)picolinamide;
5-[3-[2-(2,6-dioxo-3-piperidyl)-1-oxo-isoindolin-4-yl]prop-2-ynoxy]-N-[2-(1-methylpyrrolidin-2-yl)-1H-pyrrolo[3,2-c]pyridin-6-yl]pyridine-2-carboxamide;
4-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)benzamide;
4-(4-(2-(2,6-dioxopiperidin-3-yl)-1-oxoisoindolin-4-yl)but-3-yn-1-yl)-N-(2-((R)-1-methylpyrrolidin-2-yl)-1H-pyrrolo[3,2-c]pyridin-6-yl)benzamide;
4-(4-(1-(2,6-dioxopiperidin-3-yl)-3-methyl-2-oxo-2,3-dihydro-1H-benzo[d]imidazol-4-yl)but-3-yn-1-yl)-N-(2-((R)-1-methylpyrrolidin-2-yl)-1H-pyrrolo[3,2-c]pyridin-6-yl)benzamide;
4-(4-(2-(2,6-dioxopiperidin-3-yl)-1-oxoisoindolin-4-yl) but-3-yn-1-yl)-2-fluoro-N-(2-((R)-1-methylpyrrolidin-2-yl)-1H-pyrrolo[3,2-c]pyridin-6-yl)benzamide;
4-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
4-((5-(2-(2,6-dioxopiperidin-3-yl)-1-oxoisoindolin-4-yl)pent-4-yn-1-yl)oxy)-N-(2-((R)-1-methylpyrrolidin-2-yl)-1H-pyrrolo[3,2-c]pyridin-6-yl)benzamide;
4-((3-(2-(2,6-dioxopiperidin-3-yl)-1-oxoisoindolin-4-yl)prop-2-yn-1-yl)oxy)-2-fluoro-N-(2-((R)-1-methylpyrrolidin-2-yl)-1H-pyrrolo[3,2-c]pyridin-6-yl)benzamide;
3-((3-(2-(2,6-dioxopiperidin-3-yl)-1-oxoisoindolin-4-yl)prop-2-yn-1-yl)oxy)-N-(2-((R)-1-methylpyrrolidin-2-yl)-1H-pyrrolo[3,2-c]pyridin-6-yl)benzamide;
4-(1-(6-(2-(2,6-dioxopiperidin-3-yl)-1-oxoisoindolin-4-yl)hex-5-yn-1-yl)-1H-pyrazol-4-yl)-N-(2-((R)-1-methylpyrrolidin-2-yl)-1H-pyrrolo[3,2-c]pyridin-6-yl)benzamide;
N—((S)-2,6-dioxopiperidin-3-yl)-6-(4-(3-(4-(4-((2-((R)-1-methylpyrrolidin-2-yl)-1H-pyrrolo[3,2-c]pyridin-6-yl)carbamoyl)phenyl)-1H-pyrazol-1-yl)propyl)piperidin-1-yl)picolinamide;
N—((S)-2,6-dioxopiperidin-3-yl)-6-(4-(2-(4-(4-((2-((R)-1-methylpyrrolidin-2-yl)-1H-pyrrolo[3,2-c]pyridin-6-yl)carbamoyl)phenyl)-1H-pyrazol-1-yl)ethoxy)piperidin-1-yl)picolinamide;
N—((S)-2,6-dioxopiperidin-3-yl)-6-(5-(4-(4-((2-((R)-1-methylpyrrolidin-2-yl)-1H-pyrrolo[3,2-c]pyridin-6-yl)carbamoyl)phenyl)-1H-pyrazol-1-yl)pent-1-yn-1-yl) picolinamide;
(R)-4-(5-(6-(2,4-dioxotetrahydropyrimidin-1(2H)-yl)pyridin-2-yl)pent-4-yn-1-yl)-N-(2-(1-methylpyrrolidin-2-yl)-1H-pyrrolo[3,2-c]pyridin-6-yl)benzamide;
6-(4-(4-(2-(2,6-dioxopiperidin-3-yl)-1,3-dioxoisoindolin-4-yl)piperazin-1-yl)piperidin-1-yl)-N-(2-((R)-1-methylpyrrolidin-2-yl)-1H-pyrrolo[3,2-c]pyridin-6-yl)nicotinamide;
2-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)pyrimidine-5-carboxamide;
4-((3-(1-(2,6-dioxopiperidin-3-yl)-3-methyl-2-oxo-2,3-dihydro-TH-benzo[d]imidazol-4-yl)prop-2-yn-1-yl)oxy)-N-(2-((R)-1-methylpyrrolidin-2-yl)-1H-pyrrolo[3,2-c]pyridin-6-yl)benzamide;
6-(4-(4-(2-(2,6-dioxopiperidin-3-yl)-1-oxoisoindolin-4-yl)phenyl)piperidin-1-yl)-N-(2-((R)-1-methylpyrrolidin-2-yl)-1H-pyrrolo[3,2-c]pyridin-6-yl)nicotinamide;
7-{[2-(2,6-dioxopiperidin-3-yl)-1,3-dioxoisoindol-4-yl]amino}-N-{2-[(2R)-1-methylpyrrolidin-2-yl]-1H-pyrrolo[3,2-c]pyridin-6-yl}heptanamide;
(2S,4R)-1-[(2S)-3,3-dimethyl-2-(11-{[4-({2-[(2R)-1-methylpyrrolidin-2-yl]-1H-pyrrolo[3,2-c]pyridin-6-yl}carbamoyl)phenyl]formamido}undecanamido)butanoyl]-4-hydroxy-N-{[4-(4-methyl-1,3-thiazol-5-yl)phenyl]methyl}pyrrolidine-2-carboxamide;
(2S,4R)-1-[(2S)-3,3-dimethyl-2-(11-{[4-({2-[(2S)-1-methylpyrrolidin-2-yl]-1H-pyrrolo[3,2-c]pyridin-6-yl}carbamoyl)phenyl]formamido}undecanamido)butanoyl]-4-hydroxy-N-{[4-(4-methyl-1,3-thiazol-5-yl)phenyl]methyl}pyrrolidine-2-carboxamide;
N-(3-chloro-2-((R)-1-methylpyrrolidin-2-yl)-1H-pyrrolo[3,2-c]pyridin-6-yl)-6-(6-(2-(2,6-dioxopiperidin-3-yl)-1-oxoisoindolin-4-yl)hex-5-yn-1-yl)nicotinamide;
N-(3-chloro-2-((R)-1-methylpyrrolidin-2-yl)-1H-pyrrolo[3,2-c]pyridin-6-yl)-6-(4-(3-(2-(2,6-dioxopiperidin-3-yl)-1-oxoisoindolin-4-yl)prop-2-yn-1-yl)piperazin-1-yl)nicotinamide;
6-(4-(3-(2-(2,6-dioxopiperidin-3-yl)-1-oxoisoindolin-4-yl)prop-2-yn-1-yl)piperidin-1-yl)-N-(2-((R)-1-ethylpyrrolidin-2-yl)-1H-pyrrolo[3,2-c]pyridin-6-yl)nicotinamide;
6-(6-(2-(2,6-dioxopiperidin-3-yl)-1-oxoisoindolin-4-yl)hex-5-yn-1-yl)-N-(2-((R)-1-ethylpyrrolidin-2-yl)-1H-pyrrolo[3,2-c]pyridin-6-yl)nicotinamide;
6-(1′-(2-(2,6-dioxopiperidin-3-yl)-1-oxoisoindolin-4-yl)-[4,4′-bipiperidin]-1-yl)-N-(2-((R)-1-methylpyrrolidin-2-yl)-1H-pyrrolo[3,2-c]pyridin-6-yl)nicotinamide;
4-(3-(1-(2-(2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide;
4-(3-(1-(2-((rel-S)-2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide;
4-(3-(1-(2-((rel-R)-2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide;
4-(2-(1-(2-((rel-S)-2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide;
4-(2-(1-(2-((rel-R)-2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1,3-dioxoisoindolin-4-yl) piperidin-4-yl) ethoxy)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1,3-dioxoisoindolin-5-yl)piperidin-4-yl)ethoxy)-N-(2-((R)-1-methylpyrrolidin-2-yl)-1H-pyrrolo[3,2-c]pyridin-6-yl)benzamide;
4-(5-(2-(2,6-dioxopiperidin-3-yl)-1-oxoisoindolin-4-yl)pent-4-yn-1-yl)-3,5-difluoro-N-(2-((R)-1-methylpyrrolidin-2-yl)-1H-pyrrolo[3,2-c]pyridin-6-yl benzamide;
4-(4-((4-(2-(2,6-dioxopiperidin-3-yl)-1-oxoisoindolin-4-yl)benzyl)oxy)piperidin-1-yl)-N-(2-((R)-1-methylpyrrolidin-2-yl)-1H-pyrrolo[3,2-c]pyridin-6-yl)benzamide;
6-(6-(2-(2,6-dioxopiperidin-3-yl)-1-oxoisoindolin-4-yl)hex-5-yn-1-yl)-N-(2-((rel-R)-5-methyl-5-azaspiro[2.4]heptan-6-yl)-1H-pyrrolo[3,2-c]pyridin-6-yl)nicotinamide;
4-((4-(2-(2,6-dioxopiperidin-3-yl)-1-oxoisoindolin-4-yl)but-3-yn-1-yl) (methyl)amino)-N-(2-((R)-1-methylpyrrolidin-2-yl)-1H-pyrrolo[3,2-c]pyridin-6-yl)benzamide;
4-(4-(2-(1-(2-(2,6-dioxopiperidin-3-yl)-1-oxoisoindolin-4-yl)piperidin-4-yl)acetyl) piperazin-1-yl)-N-(2-((R)-1-methylpyrrolidin-2-yl)-1H-pyrrolo[3,2-c]pyridin-6-yl)benzamide;
2-(4-(3-(2-(2,6-dioxopiperidin-3-yl)-1-oxoisoindolin-4-yl)prop-2-yn-1-yl)piperazin-1-yl)-N-(2-((R)-1-methylpyrrolidin-2-yl)-1H-pyrrolo[3,2-c]pyridin-6-yl)pyrimidine-5-carboxamide;
4-(5-(2-(2,6-dioxopiperidin-3-yl)-7-fluoro-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
4-(4-((4-(2-(2,6-dioxopiperidin-3-yl)-1-oxoisoindolin-4-yl)piperazin-1-yl)methyl)piperidin-1-yl)-N-(2-((R)-1-methylpyrrolidin-2-yl)-1H-pyrrolo[3,2-c]pyridin-6-yl)benzamide;
4-(5-(2-(2,6-dioxopiperidin-3-yl)-1-oxoisoindolin-4-yl)pent-4-yn-1-yl)-3-fluoro-N-(2-((R)-1-methylpyrrolidin-2-yl)-1H-pyrrolo[3,2-c]pyridin-6-yl)benzamide;
4-(5-(2-(2,6-dioxopiperidin-3-yl)-1-oxoisoindolin-4-yl)pent-4-yn-1-yl)-2-fluoro-N-(2-((R)-1-methylpyrrolidin-2-yl)-1H-pyrrolo[3,2-c]pyridin-6-yl)benzamide;
3-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
3-(6-(2-(2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide;
N—((S)-2,6-dioxopiperidin-3-yl)-6-(5-(4-((2-((R)-1-methylpyrrolidin-2-yl)-1H-pyrrolo[3,2-c]pyridin-6-yl)carbamoyl)phenyl)pent-1-yn-1-yl)picolinamide;
4-(4-(4-(2-(2,6-dioxopiperidin-3-yl)-1-oxoisoindolin-4-yl)benzyl)piperazin-1-yl)-N-(2-((R)-1-methylpyrrolidin-2-yl)-1H-pyrrolo[3,2-c]pyridin-6-yl)benzamide;
6-(4-(4-(2-(2,6-dioxopiperidin-3-yl)-1-oxoisoindolin-4-yl)-3-methylphenyl)piperidin-1-yl)-N-(2-((R)-1-methylpyrrolidin-2-yl)-1H-pyrrolo[3,2-c]pyridin-6-yl)nicotinamide;
N-(3-chloro-2-((R)-1-methylpyrrolidin-2-yl)-1H-pyrrolo[3,2-c]pyridin-6-yl)-4-(5-(2-(2,6-dioxopiperidin-3-yl)-1-oxoisoindolin-4-yl)pent-4-yn-1-yl)benzamide;
4-(5-(2-(2,6-dioxopiperidin-3-yl)-6-fluoro-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
4-(5-(2-((rel-S)-2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
4-(5-(2-((rel-R)-2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)benzamide;
4-(6-(2-((rel-S)-2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide;
4-(6-(2-((rel-R)-2,6-dioxopiperidin-3-yl)-1-oxoisoindolin-4-yl)hex-5-yn-1-yl)-N-(2-((R)-1-methylpyrrolidin-2-yl)-1H-pyrrolo[3,2-c]pyridin-6-yl)benzamide;
4-(5-(2-(2,6-dioxopiperidin-3-yl)-6-fluoro-1-oxoisoindolin-5-yl)pent-4-yn-1-yl)-N-(2-((R)-1-methylpyrrolidin-2-yl)-1H-pyrrolo[3,2-c]pyridin-6-yl)benzamide;
4-(1-(2-(1-(2-(2,6-dioxopiperidin-3-yl)-1-oxoisoindolin-4-yl)piperidin-4-yl)acetyl)piperidin-4-yl)-N-(2-((R)-1-methylpyrrolidin-2-yl)-1H-pyrrolo[3,2-c]pyridin-6-yl)benzamide;
6-(4-(2-(1-(2-(2,6-dioxopiperidin-3-yl)-1-oxoisoindolin-4-yl)piperidin-4-yl)acetyl)piperazin-1-yl)-N-(2-((R)-1-methylpyrrolidin-2-yl)-1H-pyrrolo[3,2-c]pyridin-6-yl)nicotinamide;
6-(4-((4-(2-(2,6-dioxopiperidin-3-yl)-1-oxoisoindolin-4-yl)benzyl)oxy)piperidin-1-yl)-N-(2-((R)-1-methylpyrrolidin-2-yl)-1H-pyrrolo[3,2-c]pyridin-6-yl)nicotinamide;
4-(5-(2-(2,6-dioxopiperidin-3-yl)-1,3-dioxoisoindolin-4-yl)pent-4-yn-1-yl)-N-(2-((R)-1-methylpyrrolidin-2-yl)-1H-pyrrolo[3,2-c]pyridin-6-yl)benzamide;
4-(5-(2-(2,6-dioxopiperidin-3-yl)-1-oxoisoindolin-4-yl)pent-4-yn-1-yl)-N-(2-((R)-1-(methyl-d3)pyrrolidin-2-yl)-1H-pyrrolo[3,2-c]pyridin-6-yl)benzamide;
4-(6-(2-(2,6-dioxopiperidin-3-yl)-1-oxoisoindolin-4-yl)hex-5-yn-1-yl)-2-fluoro-N-(2-((R)-1-methylpyrrolidin-2-yl)-1H-pyrrolo[3,2-c]pyridin-6-yl)benzamide;
N-(3-chloro-2-((R)-1-methylpyrrolidin-2-yl)-1H-pyrrolo[3,2-c]pyridin-6-yl)-4-(6-(2-(2,6-dioxopiperidin-3-yl)-1-oxoisoindolin-4-yl)hex-5-yn-1-yl)-2-fluorobenzamide;
4-(6-(2-(2,6-dioxopiperidin-3-yl)-6-fluoro-1-oxoisoindolin-5-yl)hex-5-yn-1-yl)-2-fluoro-N-(2-((R)-1-methylpyrrolidin-2-yl)-1H-pyrrolo[3,2-c]pyridin-6-yl)benzamide;
4-(6-(2-(2,6-dioxopiperidin-3-yl)-1-oxoisoindolin-4-yl)hex-5-yn-1-yl)-2-methoxy-N-(2-((R)-1-methylpyrrolidin-2-yl)-1H-pyrrolo[3,2-c]pyridin-6-yl)benzamide;
4-(4-((1-(2-(2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)benzamide;
4-(4-((1-(2-((rel-S)-2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)benzamide;
4-(4-((1-(2-((rel-R)-2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)benzamide;
6-(1-((1-(2-(2,6-dioxopiperidin-3-yl)-1-oxoisoindolin-4-yl)piperidin-4-yl)methyl)piperidin-4-yl)-N-(2-((R)-1-methylpyrrolidin-2-yl)-1H-pyrrolo[3,2-c]pyridin-6-yl)nicotinamide;
4-(4-((1-(2-(2,6-dioxopiperidin-3-yl)-1-oxoisoindolin-4-yl)piperidin-4-yl)methyl)piperazin-1-yl)-2-fluoro-6-methoxy-N-(2-((R)-1-methylpyrrolidin-2-yl)-1H-pyrrolo[3,2-c]pyridin-6-yl)benzamide;
4-((1-((1-(2-(2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
4-((1-((1-(2-((rel-S)-2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
4-((1-((1-(2-((rel-R)-2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
4-((1-((1-(2-(2,6-dioxopiperidin-3-yl)-1,3-dioxoisoindolin-5-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
6-((1-((1-(2-(2,6-dioxopiperidin-3-yl)-1-oxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)nicotinamide;
6-(4-((1-(2-(2,6-dioxopiperidin-3-yl)-1-oxoisoindolin-4-yl)piperidin-4-yl)methyl)piperazin-1-yl)-2-methoxy-N-(2-((R)-1-methylpyrrolidin-2-yl)-1H-pyrrolo[3,2-c]pyridin-6-yl)nicotinamide;
6-((2S)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)nicotinamide;
4-(3-(1-(2-(2,6-dioxopiperidin-3-yl)-1,3-dioxoisoindolin-4-yl)piperidin-4-yl)propyl)-N-(2-((R)-1-methylpyrrolidin-2-yl)-1H-pyrrolo[3,2-c]pyridin-6-yl)benzamide;
6-(4-(4-(2-(2,6-dioxopiperidin-3-yl)-1-oxoisoindolin-4-yl)benzyl)piperazin-1-yl)-N-(2-((R)-1-methylpyrrolidin-2-yl)-1H-pyrrolo[3,2-c]pyridin-6-yl)nicotinamide;
4-(3-(4-(2-(2,6-dioxopiperidin-3-yl)-1,3-dioxoisoindolin-4-yl)piperazin-1-yl)propyl)-N-(2-((R)-1-methylpyrrolidin-2-yl)-1H-pyrrolo[3,2-c]pyridin-6-yl)benzamide;
4-(3-(4-(2-(2,6-dioxopiperidin-3-yl)-1-oxoisoindolin-4-yl)piperazin-1-yl)propyl)-N-(2-((R)-1-methylpyrrolidin-2-yl)-1H-pyrrolo[3,2-c]pyridin-6-yl)benzamide;
4-((2S)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide;
4-((2R)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide;
4-((R)-4-((1-(2-((rel-R)-2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide;
4-((R)-4-((1-(2-((rel-S)-2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)benzamide;
4-(4-((1-(2-(2,6-dioxopiperidin-3-yl)-1,3-dioxoisoindolin-4-yl)piperidin-4-yl)oxy)piperidin-1-yl)-N-(2-((R)-1-methylpyrrolidin-2-yl)-1H-pyrrolo[3,2-c]pyridin-6-yl)benzamide;
4-(4-((1-(2-(2,6-dioxopiperidin-3-yl)-1-oxoisoindolin-4-yl)piperidin-4-yl)oxy)piperidin-1-yl)-N-(2-((R)-1-methylpyrrolidin-2-yl)-1H-pyrrolo[3,2-c]pyridin-6-yl)benzamide;
6-(4-(2-(4-(2-(2,6-dioxopiperidin-3-yl)-1-oxoisoindolin-4-yl)piperazin-1-yl)ethyl)piperidin-1-yl)-N-(2-((R)-1-methylpyrrolidin-2-yl)-1H-pyrrolo[3,2-c]pyridin-6-yl)nicotinamide;
6-(4-((1-(2-(2,6-dioxopiperidin-3-yl)-1-oxoisoindolin-5-yl)piperidin-4-yl)oxy)piperidin-1-yl)-N-(2-((R)-1-methylpyrrolidin-2-yl)-1H-pyrrolo[3,2-c]pyridin-6-yl)nicotinamide;
6-(4-(((1-(2-(2,6-dioxopiperidin-3-yl)-1-oxoisoindolin-4-yl)piperidin-4-yl)oxy)methyl)piperidin-1-yl)-N-(2-((R)-1-methylpyrrolidin-2-yl)-1H-pyrrolo[3,2-c]pyridin-6-yl)nicotinamide;
4-(2-(4-(2-(2,6-dioxopiperidin-3-yl)-1-oxoisoindolin-4-yl)piperidin-1-yl)ethoxy)-N-(2-((R)-1-methylpyrrolidin-2-yl)-1H-pyrrolo[3,2-c]pyridin-6-yl)benzamide;
4-(2-(4-(2-(2,6-dioxopiperidin-3-yl)-1-oxoisoindolin-4-yl)piperazin-1-yl)ethoxy)-N-(2-((R)-1-methylpyrrolidin-2-yl)-1H-pyrrolo[3,2-c]pyridin-6-yl)benzamide;
4-((1-((1-(2-(2,6-dioxopiperidin-3-yl)-1-oxoisoindolin-5-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
4-((1-((1-(2-(2,6-dioxopiperidin-3-yl)-1,3-dioxoisoindolin-4-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
6-(4-((1-(2-(2,6-dioxopiperidin-3-yl)-1-oxoisoindolin-4-yl)piperidin-4-yl)oxy)piperidin-1-yl)-N-(2-((R)-1-methylpyrrolidin-2-yl)-1H-pyrrolo[3,2-c]pyridin-6-yl)nicotinamide;
4-(4-((1-(2-(2,6-dioxopiperidin-3-yl)-1-oxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)benzamide;
6-(4-((1-(2-(2,6-dioxopiperidin-3-yl)-1-oxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)nicotinamide;
6-[4-[[1-[2-(2,6-dioxo-3-piperidyl)-1,3-dioxo-isoindolin-4-yl]-4-piperidyl]oxy]-1-piperidyl]-N-[2-[(2R)-1-methylpyrrolidin-2-yl]-1H-pyrrolo[3,2-c]pyridin-6-yl]pyridine-3-carboxamide;
6-(4-(5-(2-(2,6-dioxopiperidin-3-yl)-1-oxoisoindolin-4-yl)pent-4-yn-1-yl)piperazin-1-yl)-N-(2-((R)-1-methylpyrrolidin-2-yl)-1H-pyrrolo[3,2-c]pyridin-6-yl)nicotinamide;
6-((5-(2-(2,6-dioxopiperidin-3-yl)-1-oxoisoindolin-4-yl)pent-4-yn-1-yl)oxy)-N-(2-((R)-1-methylpyrrolidin-2-yl)-1H-pyrrolo[3,2-c]pyridin-6-yl)nicotinamide;
4-(4-((1-(2-(2,6-dioxopiperidin-3-yl)-1,3-dioxoisoindolin-4-yl)piperidin-4-yl)methoxy)piperidin-1-yl)-N-(2-((R)-1-methylpyrrolidin-2-yl)-1H-pyrrolo[3,2-c]pyridin-6-yl)benzamide;
6-(1′-(2-(2,6-dioxopiperidin-3-yl)-1,3-dioxoisoindolin-4-yl)-[4,4′-bipiperidin]-1-yl)-N-(2-((R)-1-methylpyrrolidin-2-yl)-1H-pyrrolo[3,2-c]pyridin-6-yl)nicotinamide;
6-(4-(3-(2-(2,6-dioxopiperidin-3-yl)-1-oxoisoindolin-4-yl)prop-2-yn-1-yl)piperidin-1-yl)-N-(2-((R)-1-methylpyrrolidin-2-yl)-1H-pyrrolo[3,2-c]pyridin-6-yl)pyridazine-3-carboxamide;
5-(4-(3-(2-(2,6-dioxopiperidin-3-yl)-1-oxoisoindolin-4-yl)prop-2-yn-1-yl)piperidin-1-yl)-N-(2-((R)-1-methylpyrrolidin-2-yl)-1H-pyrrolo[3,2-c]pyridin-6-yl)pyrazine-2-carboxamide;
4-(4-(3-(2-(2,6-dioxopiperidin-3-yl)-1-oxoisoindolin-4-yl)prop-2-yn-1-yl)piperazin-1-yl)-2-fluoro-N-(2-((R)-1-methylpyrrolidin-2-yl)-1H-pyrrolo[3,2-c]pyridin-6-yl)benzamide;
4-(6-(2-(2,6-dioxopiperidin-3-yl)-1-oxoisoindolin-4-yl)hex-5-yn-1-yl)-3,5-difluoro-N-(2-((R)-1-methylpyrrolidin-2-yl)-1H-pyrrolo[3,2-c]pyridin-6-yl)benzamide;
4-((1-((1-(2-(2,6-dioxopiperidin-3-yl)-1,3-dioxoisoindolin-5-yl)piperidin-4-yl)methyl)azetidin-3-yl)oxy)-N-(2-((R)-1-methylpyrrolidin-2-yl)-1H-pyrrolo[3,2-c]pyridin-6-yl)benzamide;
6-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)nicotinamide;
4-((1-(2-(2,6-dioxopiperidin-3-yl)-1-oxoisoindolin-4-yl)piperidin-4-yl)methoxy)-N-(2-((R)-1-methylpyrrolidin-2-yl)-1H-pyrrolo[3,2-c]pyridin-6-yl)benzamide;
N-(3-chloro-2-((R)-1-methylpyrrolidin-2-yl)-1H-pyrrolo[3,2-c]pyridin-6-yl)-4-(2-(1-(2-(2,6-dioxopiperidin-3-yl)-1-oxoisoindolin-4-yl)piperidin-4-yl)ethoxy)benzamide;
N-(3-chloro-2-((R)-1-methylpyrrolidin-2-yl)-1H-pyrrolo[3,2-c]pyridin-6-yl)-4-(2-(1-(2-((rel-S)-2,6-dioxopiperidin-3-yl)-1-oxoisoindolin-4-yl)piperidin-4-yl)ethoxy)benzamide;
N-(3-chloro-2-((R)-1-methylpyrrolidin-2-yl)-1H-pyrrolo[3,2-c]pyridin-6-yl)-4-(2-(1-(2-((rel-R)-2,6-dioxopiperidin-3-yl)-1-oxoisoindolin-4-yl)piperidin-4-yl)ethoxy)benzamide;
6-((2R)-4-((1-(2-(2,6-dioxopiperidin-3-yl)-1-oxoisoindolin-4-yl)piperidin-4-yl)methyl)-2-methylpiperazin-1-yl)-N-(2-((R)-1-methylpyrrolidin-2-yl)-1H-pyrrolo[3,2-c]pyridin-6-yl)nicotinamide;
4-(2-(9-(2-(2,6-dioxopiperidin-3-yl)-1-oxoisoindolin-5-yl)-3,9-diazaspiro[5.5]undecan-3-yl)ethoxy)-N-(2-((R)-1-methylpyrrolidin-2-yl)-1H-pyrrolo[3,2-c]pyridin-6-yl)benzamide;
4-(2-(9-(2-(2,6-dioxopiperidin-3-yl)-1-oxoisoindolin-4-yl)-3,9-diazaspiro[5.5]undecan-3-yl)ethoxy)-N-(2-((R)-1-methylpyrrolidin-2-yl)-1H-pyrrolo[3,2-c]pyridin-6-yl)benzamide;
4-(2-(9-(2-(2,6-dioxopiperidin-3-yl)-1,3-dioxoisoindolin-5-yl)-3,9-diazaspiro[5.5]undecan-3-yl)ethoxy)-N-(2-((R)-1-methylpyrrolidin-2-yl)-1H-pyrrolo[3,2-c]pyridin-6-yl)benzamide;
4-(1-((1-(2-(2,6-dioxopiperidin-3-yl)-1-oxoisoindolin-4-yl)piperidin-4-yl)methyl)piperidin-4-yl)-N-(2-((R)-1-methylpyrrolidin-2-yl)-1H-pyrrolo[3,2-c]pyridin-6-yl)benzamide;
6-(4-((1-(2-(2,6-dioxopiperidin-3-yl)-1-oxoisoindolin-4-yl)piperidin-4-yl)methyl)piperazin-1-yl)-N-(2-((R)-1-methylpyrrolidin-2-yl)-1H-pyrrolo[3,2-c]pyridin-6-yl)nicotinamide;
4-(4-((1-(1-(2,6-dioxopiperidin-3-yl)-3-methyl-2-oxo-2,3-dihydro-1H-benzo[d]imidazol-5-yl)piperidin-4-yl)methyl)piperazin-1-yl)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide;
4-(2-(1-(2-((rel-S)-2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide;
4-(2-(1-(2-((rel-R)-2,6-dioxopiperidin-3-yl)-1-oxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide;
4-(3-(1-(2-(2,6-dioxopiperidin-3-yl)-1-oxoisoindolin-4-yl)piperidin-4-yl)propyl)-2-fluoro-N-(2-((R)-1-methylpyrrolidin-2-yl)-1H-pyrrolo[3,2-c]pyridin-6-yl)benzamide;
4-(3-(1-(2-((rel-S)-2,6-dioxopiperidin-3-yl)-1-oxoisoindolin-4-yl)piperidin-4-yl)propyl)-2-fluoro-N-(2-((R)-1-methylpyrrolidin-2-yl)-1H-pyrrolo[3,2-c]pyridin-6-yl)benzamide;
4-(3-(1-(2-((rel-R)-2,6-dioxopiperidin-3-yl)-1-oxoisoindolin-4-yl)piperidin-4-yl)propyl)-2-fluoro-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1-oxoisoindolin-4-yl)piperidin-4-yl)ethyl)-N-(2-((R)-1-methylpyrrolidin-2-yl)-1H-pyrrolo[3,2-c]pyridin-6-yl)benzamide;
6-(4-(4-(2-(2,6-dioxopiperidin-3-yl)-1,3-dioxoisoindolin-4-yl)phenyl)piperidin-1-yl)-N-(2-((R)-1-methylpyrrolidin-2-yl)-1H-pyrrolo[3,2-c]pyridin-6-yl)nicotinamide;
6-(2-(1-(2-(2,6-dioxopiperidin-3-yl)-1,3-dioxoisoindolin-4-yl)piperidin-4-yl)ethoxy)-N-(2-((R)-1-methylpyrrolidin-2-yl)-1H-pyrrolo[3,2-c]pyridin-6-yl)nicotinamide;
4-(4-((1-(2-(2,6-dioxopiperidin-3-yl)-6-fluoro-1,3-dioxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide;
N—((S)-2,6-dioxopiperidin-3-yl)-5-(4-((4-(4-((2-((R)-1-methylpyrrolidin-2-yl)-1H-pyrrolo[3,2-c]pyridin-6-yl)carbamoyl)phenyl)piperazin-1-yl)methyl)piperidin-1-yl)picolinamide;
N—((S)-2,6-dioxopiperidin-3-yl)-6-(4-((4-(4-((2-((R)-1-methylpyrrolidin-2-yl)-1H-pyrrolo[3,2-c]pyridin-6-yl)carbamoyl)phenyl)piperazin-1-yl)methyl)piperidin-1-yl)picolinamide;
4-(3-((4-(2-(2,6-dioxopiperidin-3-yl)-1,3-dioxoisoindolin-5-yl)piperazin-1-yl)methyl)azetidin-1-yl)-N-(2-((R)-1-methylpyrrolidin-2-yl)-1H-pyrrolo[3,2-c]pyridin-6-yl)benzamide;
4-((1-((1-(2-(2,6-dioxopiperidin-3-yl)-6-fluoro-1,3-dioxoisoindolin-5-yl)piperidin-4-yl)methyl)piperidin-4-yl)oxy)-N-(2-((R)-1-methylpyrrolidin-2-yl)-1H-pyrrolo[3,2-c]pyridin-6-yl)benzamide;
6-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)pyridazine-3-carboxamide;
5-(4-((2-(2,6-dioxopiperidin-3-yl)-1-oxoisoindolin-4-yl)ethynyl)piperidin-1-yl)-N-(2-((R)-1-methylpyrrolidin-2-yl)-1H-pyrrolo[3,2-c]pyridin-6-yl)pyrazine-2-carboxamide;
6-((3S)-3-(3-(2-(2,6-dioxopiperidin-3-yl)-1-oxoisoindolin-4-yl)prop-2-yn-1-yl)pyrrolidin-1-yl)-N-(2-((R)-1-methylpyrrolidin-2-yl)-1H-pyrrolo[3,2-c]pyridin-6-yl)nicotinamide;
6-(4-(6-(2-(2,6-dioxopiperidin-3-yl)-1-oxoisoindolin-4-yl)hex-5-yn-1-yl)piperidin-1-yl)-N-(2-((R)-1-methylpyrrolidin-2-yl)-1H-pyrrolo[3,2-c]pyridin-6-yl)nicotinamide;
4-(4-(3-(1-(2-(2,6-dioxopiperidin-3-yl)-1-oxoisoindolin-4-yl)piperidin-4-yl)propanoyl)piperazin-1-yl)-N-(2-((R)-1-methylpyrrolidin-2-yl)-1H-pyrrolo[3,2-c]pyridin-6-yl)benzamide;
N-(3-chloro-2-((R)-1-methylpyrrolidin-2-yl)-1H-pyrrolo[3,2-c]pyridin-6-yl)-4-(2-(1-(2-(2,6-dioxopiperidin-3-yl)-1-oxoisoindolin-4-yl)piperidin-4-yl)ethoxy)-2-fluorobenzamide;
N-(3-chloro-2-((R)-1-methylpyrrolidin-2-yl)-1H-pyrrolo[3,2-c]pyridin-6-yl)-6-(4-((1-(2-(2,6-dioxopiperidin-3-yl)-1-oxoisoindolin-4-yl)piperidin-4-yl)methoxy)piperidin-1-yl)nicotinamide;
3-(4-((1-(2-(2,6-dioxopiperidin-3-yl)-1-oxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide;
6-(4-(2-(1-(2-(2,6-dioxopiperidin-3-yl)-1-oxoisoindolin-4-yl)piperidin-4-yl)ethyl)piperazin-1-yl)-N-(2-((R)-1-methylpyrrolidin-2-yl)-1H-pyrrolo[3,2-c]pyridin-6-yl)nicotinamide;
4-((4-((1-(2-(2,6-dioxopiperidin-3-yl)-1-oxoisoindolin-4-yl)piperidin-4-yl)methyl)piperazin-1-yl)methyl)-N-(2-((R)-1-methylpyrrolidin-2-yl)-1H-pyrrolo[3,2-c]pyridin-6-yl)benzamide;
6-(4-((3-(2-(2,6-dioxopiperidin-3-yl)-1-oxoisoindolin-4-yl)propa-1,2-dien-1-yl)oxy)piperidin-1-yl)-N-(2-((R)-1-methylpyrrolidin-2-yl)-1H-pyrrolo[3,2-c]pyridin-6-yl)nicotinamide;
6-(4-(4-(2-(2,6-dioxopiperidin-3-yl)-1-oxoisoindolin-4-yl)-3-methylphenoxy)piperidin-1-yl)-N-(2-((R)-1-methylpyrrolidin-2-yl)-1H-pyrrolo[3,2-c]pyridin-6-yl)nicotinamide;
5-(5-(2-(2,6-dioxopiperidin-3-yl)-1-oxoisoindolin-4-yl)pent-4-yn-1-yl)-N-(2-((R)-1-methylpyrrolidin-2-yl)-1H-pyrrolo[3,2-c]pyridin-6-yl)picolinamide;
4-(3-(4-(2-(2,6-dioxopiperidin-3-yl)-1-oxoisoindolin-4-yl)piperidin-1-yl)propyl)-N-(2-((R)-1-methylpyrrolidin-2-yl)-1H-pyrrolo[3,2-c]pyridin-6-yl)benzamide;
4-(3-(4-(2-(2,6-dioxopiperidin-3-yl)-1-oxoisoindolin-4-yl)phenyl)propyl)-N-(2-((R)-1-methylpyrrolidin-2-yl)-1H-pyrrolo[3,2-c]pyridin-6-yl)benzamide;
4-((7-(2-(2,6-dioxopiperidin-3-yl)-1-oxoisoindolin-4-yl)-7-azaspiro[3.5]nonan-2-yl)oxy)-2-fluoro-N-(2-((R)-1-methylpyrrolidin-2-yl)-1H-pyrrolo[3,2-c]pyridin-6-yl)benzamide;
4-((7-(2-(2,6-dioxopiperidin-3-yl)-1-oxoisoindolin-4-yl)-7-azaspiro[3.5]nonan-2-yl)methoxy)-2-fluoro-N-(2-((R)-1-methylpyrrolidin-2-yl)-1H-pyrrolo[3,2-c]pyridin-6-yl)benzamide;
4-(9-(2-(2,6-dioxopiperidin-3-yl)-1-oxoisoindolin-4-yl)-3,9-diazaspiro[5.5]undecan-3-yl)-2-fluoro-N-(2-((R)-1-methylpyrrolidin-2-yl)-1H-pyrrolo[3,2-c]pyridin-6-yl)benzamide;
4-(4-(4-(2-(2,6-dioxopiperidin-3-yl)-1-oxoisoindolin-4-yl)-3-methylphenoxy)piperidin-1-yl)-2-fluoro-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1-oxoisoindolin-4-yl)piperidin-4-yl)ethoxy)-2-methyl-N-(2-((R)-1-methylpyrrolidin-2-yl)-1H-pyrrolo[3,2-c]pyridin-6-yl)benzamide;
2-chloro-4-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1-oxoisoindolin-4-yl)piperidin-4-yl)ethoxy)-3-fluoro-N-(2-((R)-1-methylpyrrolidin-2-yl)-1H-pyrrolo[3,2-c]pyridin-6-yl)benzamide;
4-(4-(2-(2,6-dioxopiperidin-3-yl)-1-oxoisoindolin-4-yl)phenethoxy)-2-fluoro-N-(2-((R)-1-methylpyrrolidin-2-yl)-1H-pyrrolo[3,2-c]pyridin-6-yl)benzamide;
4-(4-(2-(2,6-dioxopiperidin-3-yl)-1-oxoisoindolin-4-yl)-3-methylphenethoxy)-2-fluoro-N-(2-((R)-1-methylpyrrolidin-2-yl)-1H-pyrrolo[3,2-c]pyridin-6-yl)benzamide;
4-(3-(1-(2-(2,6-dioxopiperidin-3-yl)-1-oxoisoindolin-4-yl)piperidin-4-yl)propoxy)-2-fluoro-N-(2-((R)-1-methylpyrrolidin-2-yl)-1H-pyrrolo[3,2-c]pyridin-6-yl)benzamide;
4-(1-(3-(1-(2-(2,6-dioxopiperidin-3-yl)-1,3-dioxoisoindolin-4-yl)piperidin-4-yl)propyl)-1H-pyrazol-4-yl)-2-fluoro-N-(2-((R)-1-methylpyrrolidin-2-yl)-1H-pyrrolo[3,2-c]pyridin-6-yl)benzamide;
5-(1-(3-(1-(2-(2,6-dioxopiperidin-3-yl)-1,3-dioxoisoindolin-4-yl)piperidin-4-yl)propyl)-1H-pyrazol-4-yl)-N-(2-((R)-1-methylpyrrolidin-2-yl)-1H-pyrrolo[3,2-c]pyridin-6-yl)picolinamide;
4-(1-(3-(1-(2-(2,6-dioxopiperidin-3-yl)-1-oxoisoindolin-4-yl)piperidin-4-yl)propyl)-1H-pyrazol-4-yl)-2-fluoro-N-(2-((R)-1-methylpyrrolidin-2-yl)-1H-pyrrolo[3,2-c]pyridin-6-yl)benzamide;
4-((5-(2-(2,6-dioxopiperidin-3-yl)-1-oxoisoindolin-4-yl)pent-4-yn-1-yl)oxy)-2-fluoro-N-(2-((R)-1-methylpyrrolidin-2-yl)-1H-pyrrolo[3,2-c]pyridin-6-yl)benzamide;
4-((1-(2-(2,6-dioxopiperidin-3-yl)-1-oxoisoindolin-4-yl)piperidin-4-yl)methoxy)-2-fluoro-N-(2-((R)-1-methylpyrrolidin-2-yl)-1H-pyrrolo[3,2-c]pyridin-6-yl)benzamide;
N—((S)-2,6-dioxopiperidin-3-yl)-2-fluoro-4-(4-(2-(3-fluoro-4-((2-((R)-1-methylpyrrolidin-2-yl)-1H-pyrrolo[3,2-c]pyridin-6-yl)carbamoyl)phenoxy)ethyl)piperidin-1-yl)benzamide;
5-(2-(1-(2-(2,6-dioxopiperidin-3-yl)-1-oxoisoindolin-4-yl)piperidin-4-yl)ethoxy)-N-(2-((R)-1-methylpyrrolidin-2-yl)-1H-pyrrolo[3,2-c]pyridin-6-yl)picolinamide;
4-(2-(1-(2-(2,6-dioxopiperidin-3-yl)-1,3-dioxoisoindolin-4-yl)piperidin-4-yl)ethoxy)-2-fluoro-N-(2-((R)-1-methylpyrrolidin-2-yl)-1H-pyrrolo[3,2-c]pyridin-6-yl)benzamide;
4-(4-(4-(2-(2,6-dioxopiperidin-3-yl)-1-oxoisoindolin-4-yl)phenyl)piperidin-1-yl)-2-fluoro-N-(2-((R)-1-methylpyrrolidin-2-yl)-1H-pyrrolo[3,2-c]pyridin-6-yl)benzamide;
N—((S)-2,6-dioxopiperidin-3-yl)-2-fluoro-3-(5-(4-((2-((R)-1-methylpyrrolidin-2-yl)-1H-pyrrolo[3,2-c]pyridin-6-yl)carbamoyl)phenyl)pent-1-yn-1-yl)benzamide;
3-(4-((1-(2-(2,6-dioxopiperidin-3-yl)-1,3-dioxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide;
4-(4-((1-(2-(2,6-dioxopiperidin-3-yl)-1,3-dioxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide;
4-(4-((1-(2-(2,6-dioxopiperidin-3-yl)-1-oxoisoindolin-5-yl)piperidin-4-yl)methyl)piperazin-1-yl)-N-(2-((R)-1-methylpyrrolidin-2-yl)-1H-pyrrolo[3,2-c]pyridin-6-yl)benzamide;
4-(6-(2-(2,6-dioxopiperidin-3-yl)-1-oxoisoindolin-5-yl)hex-5-yn-1-yl)-2-fluoro-N-(2-((R)-1-methylpyrrolidin-2-yl)-1H-pyrrolo[3,2-c]pyridin-6-yl)benzamide;
6-(4-(3-(2-(2,6-dioxopiperidin-3-yl)-1-oxoisoindolin-5-yl)prop-2-yn-1-yl)piperidin-1-yl)-N-(2-((R)-1-methylpyrrolidin-2-yl)-1H-pyrrolo[3,2-c]pyridin-6-yl)nicotinamide;
4-(5-(2-(2,6-dioxopiperidin-3-yl)-3-oxoisoindolin-5-yl)pent-4-yn-1-yl)-N-(2-((R)-1-methylpyrrolidin-2-yl)-1H-pyrrolo[3,2-c]pyridin-6-yl)benzamide;
6-((3R)-3-(3-(2-(2,6-dioxopiperidin-3-yl)-1-oxoisoindolin-4-yl)prop-2-yn-1-yl)pyrrolidin-1-yl)-N-(2-((R)-1-methylpyrrolidin-2-yl)-1H-pyrrolo[3,2-c]pyridin-6-yl)nicotinamide;
4-((1-(2-(1-(2-(2,6-dioxopiperidin-3-yl)-1,3-dioxoisoindolin-5-yl)piperidin-4-yl)ethyl)azetidin-3-yl)oxy)-N-(2-((R)-1-methylpyrrolidin-2-yl)-1H-pyrrolo[3,2-c]pyridin-6-yl)benzamide;
6-(4-((1-(2-(2,6-dioxopiperidin-3-yl)-1-oxoisoindolin-5-yl)piperidin-4-yl)methoxy)piperidin-1-yl)-N-(2-((R)-1-methylpyrrolidin-2-yl)-1H-pyrrolo[3,2-c]pyridin-6-yl)nicotinamide;
N-(3-chloro-2-((R)-1-methylpyrrolidin-2-yl)-1H-pyrrolo[3,2-c]pyridin-6-yl)-4-(2-(1-(2-(2,6-dioxopiperidin-3-yl)-1-oxoisoindolin-4-yl)piperidin-4-yl)ethoxy)-3-fluorobenzamide;
4-(2-(1-(2-(2,6-dioxopiperidin-3-yl)-1-oxoisoindolin-4-yl)piperidin-4-yl)ethoxy)-2,5-difluoro-N-(2-((R)-1-methylpyrrolidin-2-yl)-1H-pyrrolo[3,2-c]pyridin-6-yl)benzamide;
4-(2-(1-(2-(2,6-dioxopiperidin-3-yl)-1-oxoisoindolin-4-yl)piperidin-4-yl)ethoxy)-2,3-difluoro-N-(2-((R)-1-methylpyrrolidin-2-yl)-1H-pyrrolo[3,2-c]pyridin-6-yl)benzamide;
4-(3-(1-(2-((S)-2,6-dioxopiperidin-3-yl)-1-oxoisoindolin-4-yl)piperidin-4-yl)propyl)-N-(2-((R)-1-methylpyrrolidin-2-yl)-1H-pyrrolo[3,2-c]pyridin-6-yl)benzamide; and
N-[3-chloro-2-[(R)-1-methylpyrrolidin-2-yl]-1H-pyrrolo[3,2-c]pyridin-6-yl]-4-[3-[1-[2-[(rel-S)-2,6-dioxo-3-piperidyl]-1-oxo-isoindolin-4-yl]-4-piperidyl]propyl]benzamide.

10. A compound according to any one of the preceding claims, wherein M is of formula (IV): 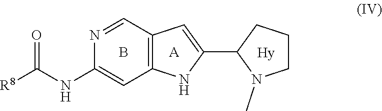
wherein
R⁸ is (i) a bond to LINK or (ii) a group selected from a 6-membered aryl ring and a 5- to 6-membered heteroaryl ring, the group R⁸ being unsubstituted or substituted by one, two or three substituents independently selected from halo, C₁₋₄ alkyl and R¹⁰;
R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl, more preferably pyrazolyl and cyclopropyl; wherein either R⁸ is (i) a bond to LINK, or R⁸ is a group (ii) and M is bonded to LINK via a C or N atom within group R⁸ such that a hydrogen atom on the C or N atom within group R⁸ is replaced with a bond to LINK.

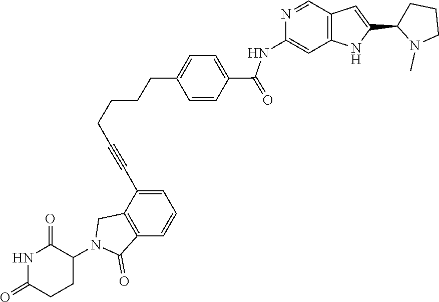 [SMILES: CN1CCC[C@@H]1c1cc2cnc(NC(=O)c3ccc(CCCCC#Cc4cccc5c4CN(C4CCC(=O)NC4=O)C5=O)cc3)cc2[nH]1]

11. A pharmaceutical composition which comprises a compound as defined in any one of claims 1 to 10 and a pharmaceutically acceptable carrier or diluent.

12. A compound as defined in any one of claims 1 to 10 for use in the treatment of the human or animal body by therapy.

13. A compound as defined in any one of claims 1 to 10 for use in the treatment of cancer.

14. The compound for use according to claim 13, wherein the cancer is a transcriptionally addicted cancer, such as breast cancer, acute leukemia, chronic leukemia, prostate cancer, bladder cancer, cholangiocarcinoma, colon adenocarcinoma, esophageal carcinoma, head and neck squamous cell carcinoma, kidney chromophobe, kidney renal clear cell carcinoma, kidney renal papillary cell carcinoma, liver hepatocellular carcinoma, lung adenocarcinoma, pheochromocytoma and paraganglioma, rectum adenocarcinoma, stomach adenocarcinoma, uterine corpus endometrial carcinoma, cervical squamous cell carcinoma and endocervical adenocarcinoma, lung squamous cell carcinoma, neuroblastoma, midline glioma or sarcoma, preferably neuroblastoma or midline glioma.

15. The compound for use according to claim 13, wherein the cancer is acute myeloid leukaemia (AML), myelodysplastic syndrome (MDS), myeloproliferative neoplasm (MPN) or acute lymphoblastic leukaemia (ALL), for instance wherein the cancer is mixed-lineage leukaemia (MLL) rearranged (also known as KMT2Ar) acute leukemia (AML and ALL), NPM1 mutant acute leukemia, RUNX1-fusion acute leukemia, E2A-fusion acute leukemia, PML-fusion acute leukemia or NUP98-fusion acute leukemia.
